(19) United States
(12) Patent Application Publication
(10) Pub. No.: US 2024/0349604 A1
(43) Pub. Date: Oct. 17, 2024

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR)

(21) Appl. No.: 18/275,390

(22) PCT Filed: May 16, 2022
(86) PCT No.: PCT/KR2022/006999

§ 371 Date: Aug. 1, 2023

(30) Foreign Application Priority Data
May 14, 2021 (KR) 10-2021-0062732
May 16, 2022 (KR) 10-2022-0059416

(51) Int. Cl. H10K 85/60; C07D 307/77; C07D 307/91; C07D 333/50; C07D 405/04; C07D 405/10; C07D 405/12; C07D 405/14; C07D 409/12; C07D 409/14; C07F 7/08; C09K 11/06; H10K 50/12; H10K 85/40; H10K 101/00

(52) U.S. Cl. CPC H10K 85/6574; C07D 307/77; C07D 307/91; C07D 333/50; C07D 405/04; C07D 405/10; C07D 405/12; C07D 405/14; C07D 409/12; C07D 409/14; C07F 7/0812; C09K 11/06; H10K 85/40; H10K 85/615; H10K 85/622; H10K 85/626; H10K 85/633; H10K 85/636; H10K 85/654; C09K 2211/1018; H10K 50/12; H10K 85/6572; H10K 85/6576; H10K 2101/90

(57) ABSTRACT

An organic light emitting device comprising an anode, a cathode, and a light emitting layer between the anode and the cathode, the light emitting layer comprising a compound of Chemical Formula 1 and a compound of Chemical Formula 2, and having improved driving voltage, efficiency and lifetime is provided.

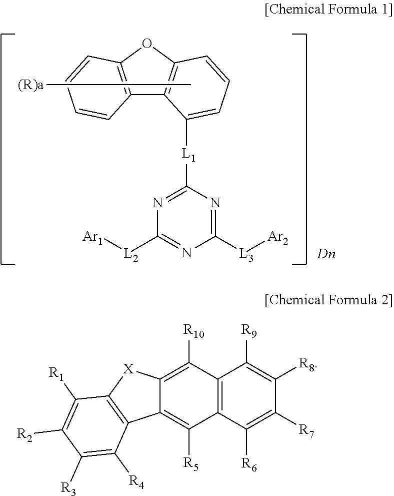

### CROSS-REFERENCE TO RELATED APPLICATION(S)

This application is a National Phase entry pursuant to 35 U.S.C. § 371 of International Application No. PCT/KR2022/006999 filed on May 16, 2022, and claims priority to and the benefit of Korean Patent Application No. 10-2021-0062732 filed on May 14, 2021 and Korean Patent Application No. 10-2022-0059416 filed on May 16, 2022, the disclosures of which are incorporated herein by reference in their entirety.

### FIELD OF DISCLOSURE

The present disclosure relates to an organic light emitting device having improved driving voltage, efficiency and lifetime.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of a new material for an organic material used in the organic light emitting device as described above.

### RELATED ART

Korean Unexamined Patent Publication No. 10-2000-0051826

### SUMMARY

It is an object of the present disclosure to provide an organic light emitting device having improved driving voltage, efficiency and lifetime.

Provided herein is the following organic light emitting device:

An organic light emitting device including: an anode, a cathode, and a light emitting layer between the anode and the cathode,
wherein the light emitting layer includes a compound represented by the following Chemical Formula 1 and a compound represented by the following Chemical Formula 2.

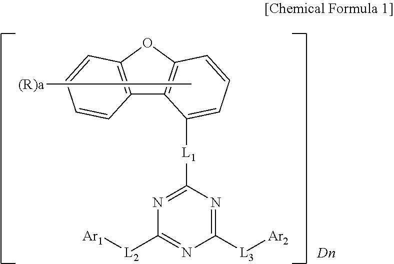

in Chemical Formula 1,
Ar₁ and Ar₂ are each independently a substituted or unsubstituted C₆₋₆₀ aryl,
L₁ to L₃ are each independently a single bond, or a substituted or unsubstituted C₆₋₆₀ arylene,
R is each independently hydrogen, deuterium, or a substituted or unsubstituted C₆₋₆₀ aryl,
Dn is a number of deuterium substitutions in the compound, where n is an integer of 0 or more, and
a is an integer of 0 to 7,

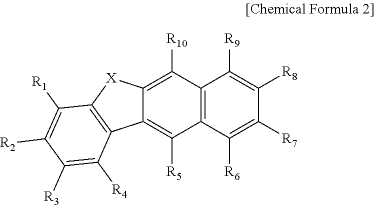

in Chemical Formula 2,
X is O or S,
one of R₁ to R₁₀ is a substituent represented by the following Chemical Formula 3, and the rest are each independently hydrogen or deuterium,

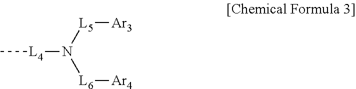

in Chemical Formula 3,
Ar₃ and Ar₄ are each independently a substituted or unsubstituted C₆₋₆₀ aryl; or a substituted or unsubstituted C₂₋₆₀ heteroaryl containing one or more selected from the group consisting of N, O and S,
L₄ is a single bond; a substituted or unsubstituted C₆₋₆₀ arylene; or a substituted or unsubstituted C₂₋₆₀ heteroarylene containing one or more selected from the group consisting of N, O and S, and
L₅ and L₆ are each independently a single bond; a substituted or unsubstituted C₆₋₆₀ arylene; or a substituted or unsubstituted C₂₋₆₀ heteroarylene containing one or more selected from the group consisting of N, O and S.

The above-mentioned organic light emitting device includes the compound represented by Chemical Formula 1 and the compound represented by Chemical Formula 2 in the light emitting layer, and thus can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

As used herein, the notation [symbol] or [symbol] means a bond linked to another substituent group.

As used herein, the term “substituted or unsubstituted” means being unsubstituted or substituted with one or more substituents selected from the substituent group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing one or more of N, O and S atoms, or being unsubstituted or substituted with a substituent from the above substituent group which is further substituted by one or more selected from the above substituent group.

In the present disclosure, the carbon number of a carbonyl group is not particularly limited, but is preferably 1 to 40. Specific examples include the following structures, but are not limited thereto.

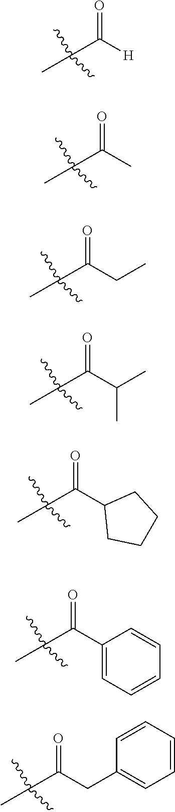

In the present disclosure, an ester group may have a structure in which oxygen of the carboxy group may be substituted by a straight-chain, branched-chain, or cyclic alkyl group having 1 to 25 carbon atoms, or an aryl group having 6 to 25 carbon atoms. Specific examples thereof include the following structures, but are not limited thereto.

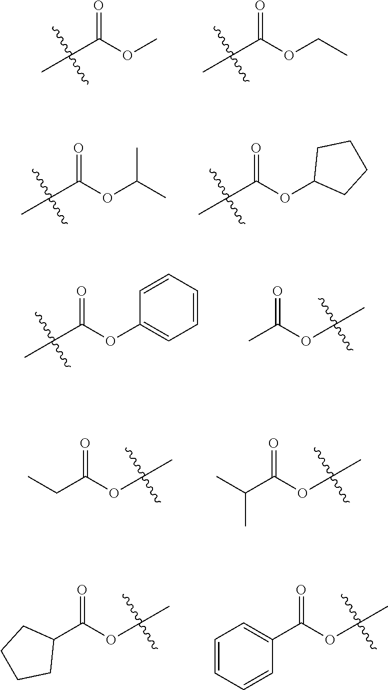

In the present disclosure, the carbon number of an imide group is not particularly limited, but is preferably 1 to 25. Specific examples thereof include the following structures, but are not limited thereto.

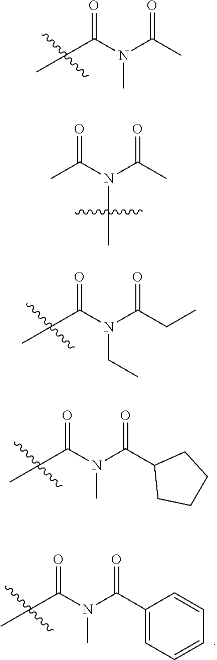

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a dimethyl boron group, a diethyl boron group, a t-butylmethyl boron group, a diphenyl boron group, and a phenyl boron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. Specifically, the carbon number of the alkenyl group is 2 to 20, or 2 to 10, or 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. Specifically, the carbon number of the cycloalkyl group is 3 to 30, or 3 to 20, or 3 to 6.

Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. Specifically, the carbon number of the aryl group is 6 to 30, or 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, or the like, but is not limited thereto.

In the present disclosure, the fluorenyl group may be substituted, and two substituents may be linked with each other to form a spiro structure. In the case where the fluorenyl group is substituted,

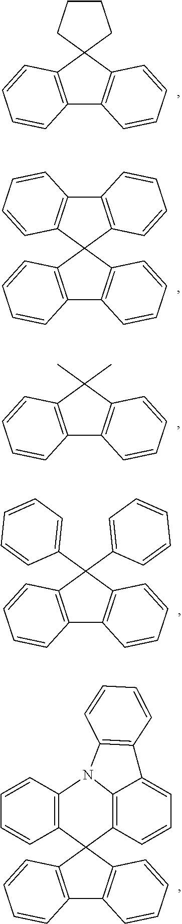

and the like can be formed. However, the structure is not limited thereto.

In the present disclosure, a heterocyclic group contains one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heterocyclic group. In the present disclosure, the alkenyl group in the aralkenyl group is the same as the aforementioned examples of the alkenyl group. In the present disclosure, the aforementioned description of the aryl group may be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

In the present disclosure, the compound represented by ‘[structural formula]_{Dn}’ is a compound of which the corresponding ‘structural formula’ is substituted with n deuteriums.

Hereinafter, the present disclosure will be described in detail for each configuration.

### Anode and Cathode

An anode and a cathode used in the present disclosure mean electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to the present disclosure may further include a hole injection layer on the anode, if necessary.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to an electron injection layer or the electron injection material, and further is excellent in the ability to form a thin film. Further, it is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive compound, and the like, but are not limited thereto.

### Hole Transport Layer

The organic light emitting device according to the present disclosure may include a hole transport layer on the anode (or on the hole injection layer if the hole injection layer exists), if necessary.

The hole transport layer is a layer that can receive the holes from the anode or the hole injection layer and transport the holes to the light emitting layer, and the hole transport material is suitably a material having large mobility to the holes, which may receive holes from the anode or the hole injection layer and transfer the holes to the light emitting layer.

Specific examples thereof include an arylamine-based organic material, a conductive polymer, a block copolymer in which a conjugate portion and a non-conjugate portion are present together, and the like, but are not limited thereto.

### Electron Blocking Layer

The electron blocking layer is a layer provided between the hole transport layer and the light emitting layer in order to prevent the electrons injected from the cathode from being transferred to the hole transport layer without being recombined in the light emitting layer, which may also be referred to as an electron inhibition layer or an electron stopping layer.

The electron blocking layer is preferably a material having a smaller electron affinity than the electron transport layer.

### Light Emitting Layer

The light emitting layer used in the present disclosure is a layer that can emit light in the visible light region by combining holes and electrons transported from the anode and the cathode. Generally, the light emitting layer includes a host material and a dopant material, and in the present disclosure, the compound represented by Chemical Formula 1 and the compound represented by Chemical Formula 2 are included as a host.

Preferably, the compound represented by Chemical Formula 1 may be represented by one of the following Chemical Formulas 1-1 to 1-3:

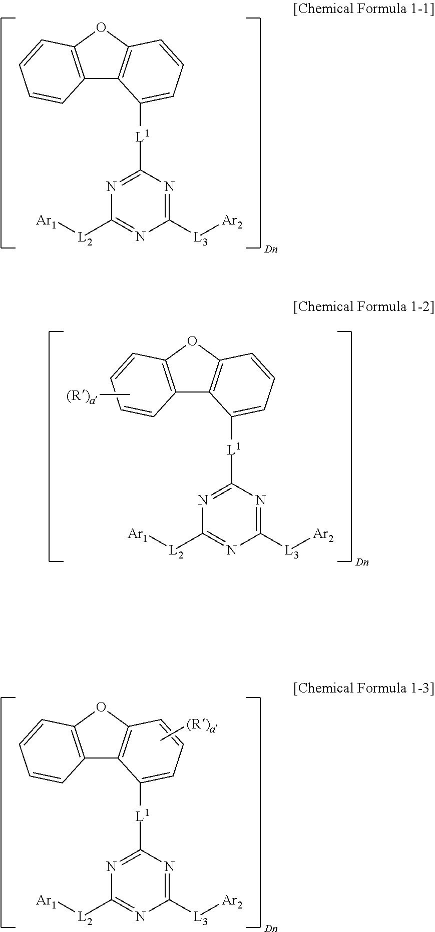

in Chemical Formulas 1-1 to 1-3,
Ar₁, Ar₂, L₁ to L₃, Dn and n are the same as defined in Chemical Formula 1,
R′ is each independently deuterium, or a substituted or unsubstituted C₆₋₆₀ aryl,
a′ is an integer of 1 to 4, and
a″ is an integer of 1 to 3.

Preferably, Ar₁ and Ar₂ may be each independently a substituted or unsubstituted C₆₋₂₀ aryl.

More preferably, Ar₁ and Ar₂ may be each independently phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl, which is substituted or unsubstituted. When the Ar₁ and Ar₂ are substituted, hydrogens of the Ar₁ and Ar₂ may be each independently substituted with deuterium; a C₁₋₂₀ alkyl such as methyl; a C₆₋₂₀ aryl such as phenyl or naphthyl; silyl; or arylsilyl such as triphenylsilyl.

More preferably, Ar₁ and Ar₂ are each independently phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl, each of which may be unsubstituted or substituted with deuterium, phenyl or triphenylsilyl.

Preferably, Ar₁ and Ar₂ may be each independently one selected from the following:

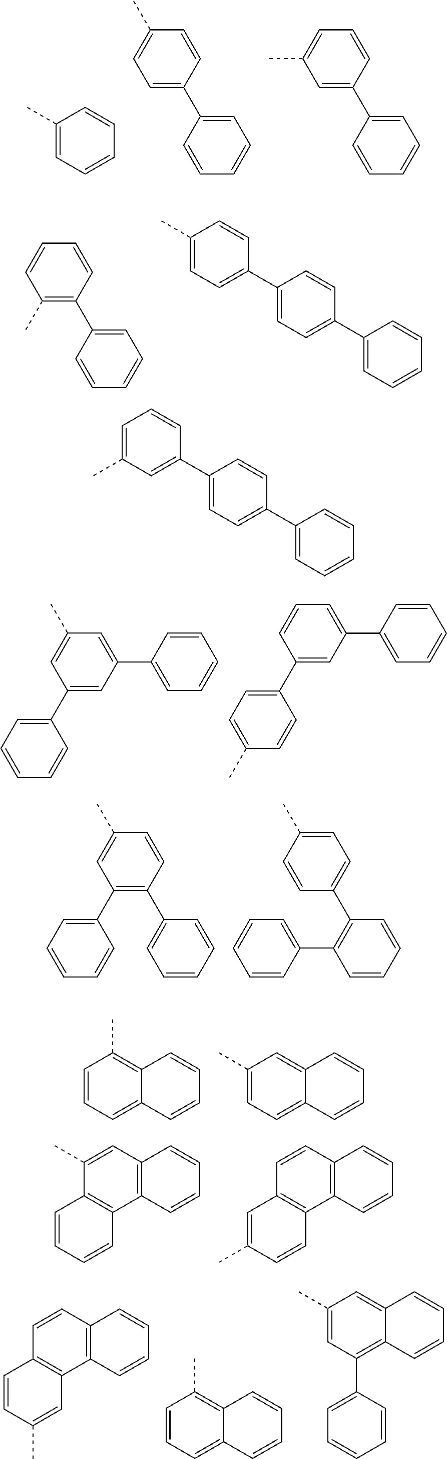

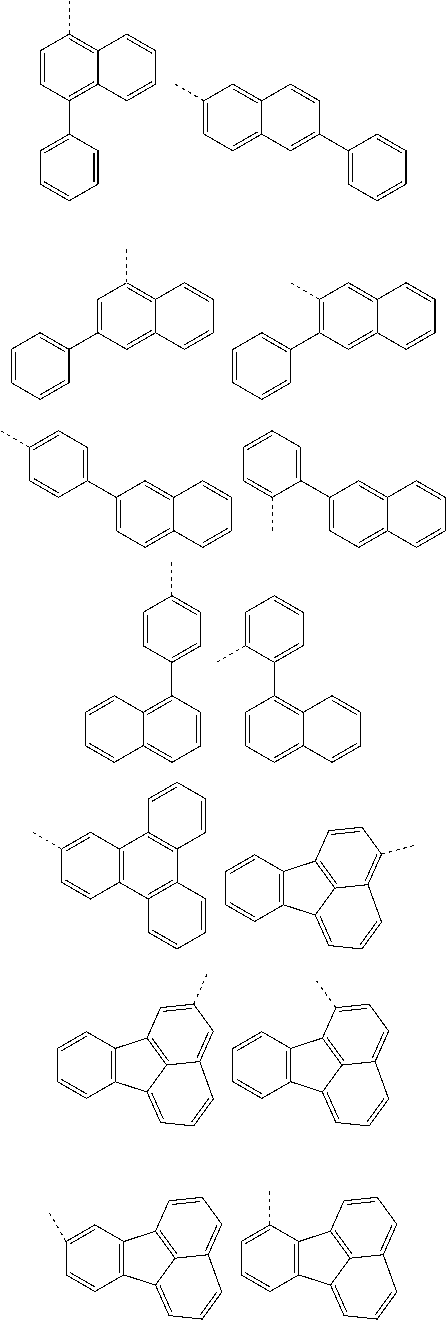

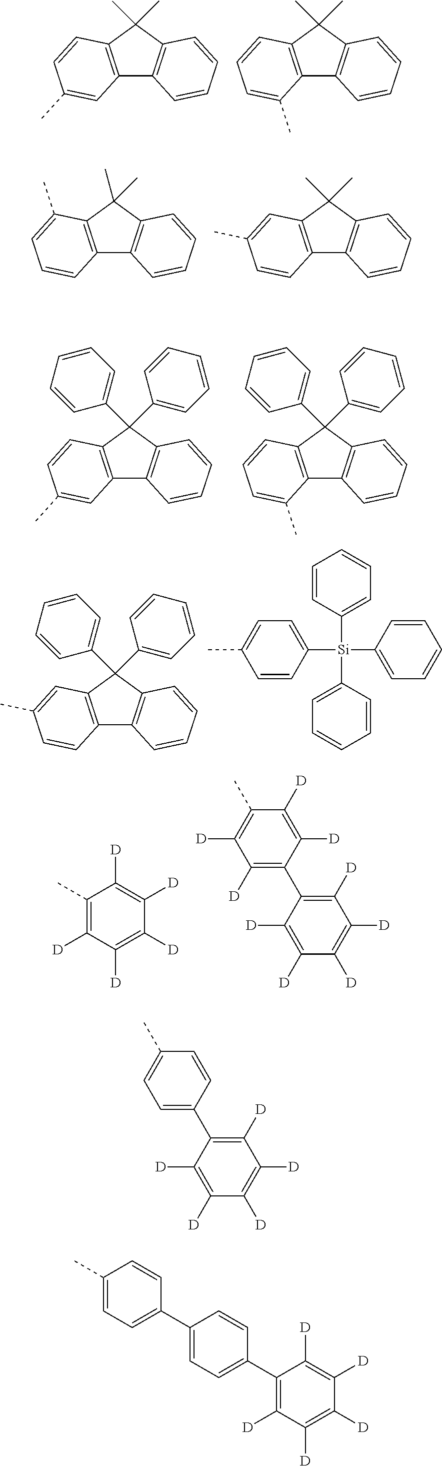

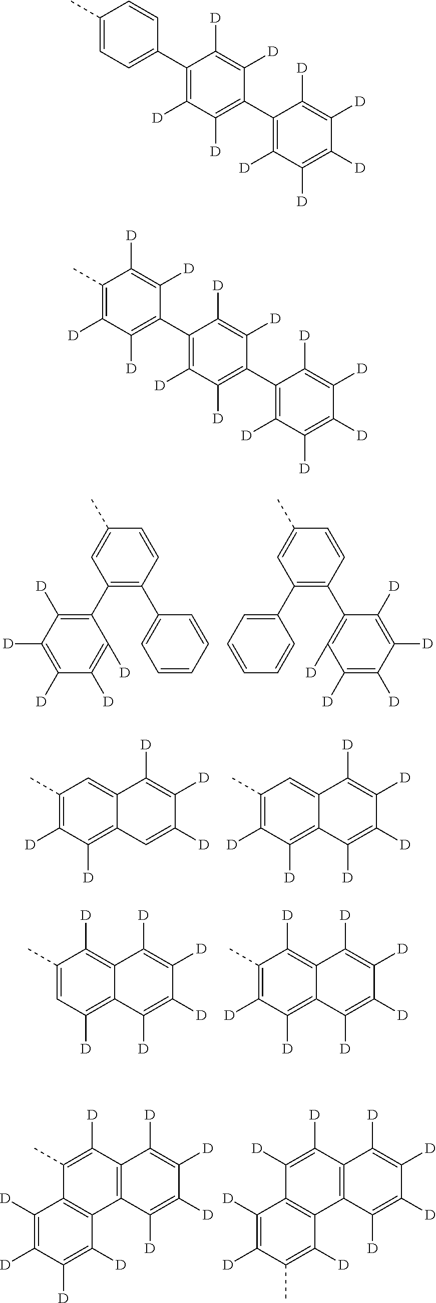

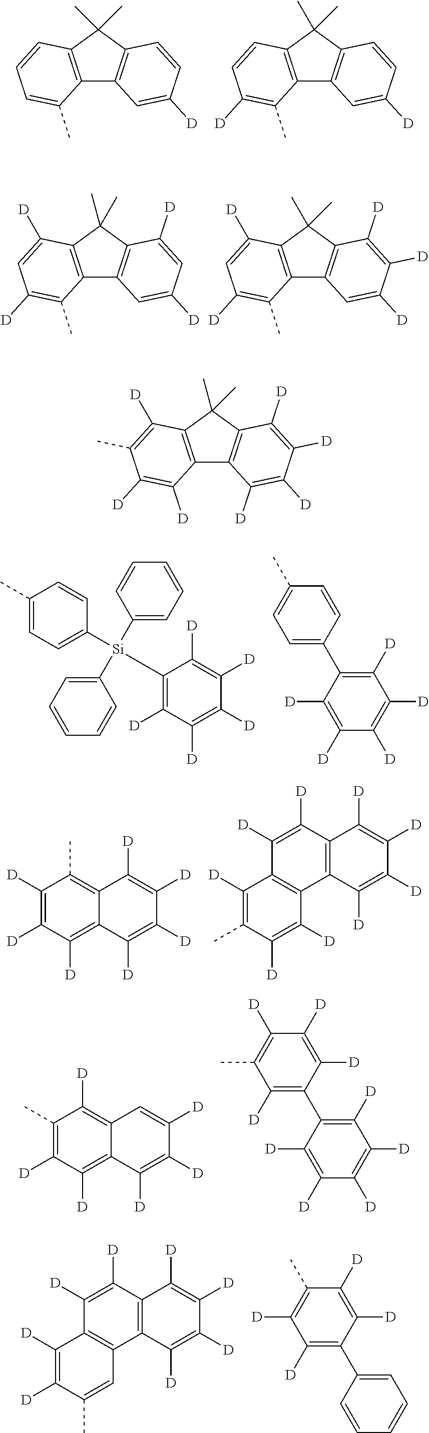

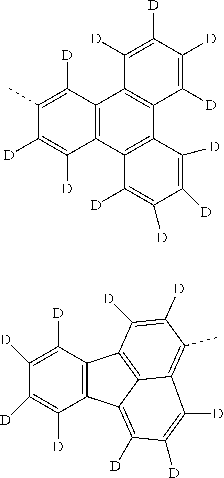

in each of the above Chemical Formulas, the dotted line represents a bonding position.

Further, the Ar₁ and Ar₂ may be the same as each other or may be different from each other.

Preferably, L₁ to L₃ may be each independently a single bond; or a substituted or unsubstituted C₆₋₂₀ arylene. Further, when the L₁ to L₃ are substituted, hydrogen of the L₁ to L₃ may be substituted with one or more of deuterium; a C₁₋₂₀ alkyl such as methyl; or a C₆₋₂₀ aryl such as phenyl or naphthyl.

More preferably, L₁ to L₃ may be each independently a single bond, phenylene, biphenyldiyl, naphthalenediyl, or binaphthalenediyl, each of which, except for a single bond, may be unsubstituted or substituted with deuterium, phenyl, or naphthyl.

Preferably, L₁ to L₃ may be each independently a single bond, or one selected from the following:

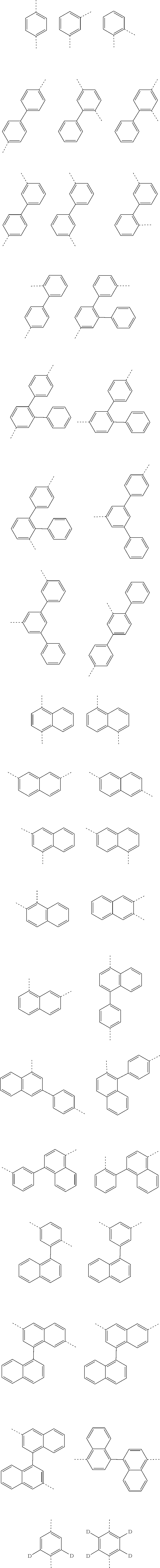

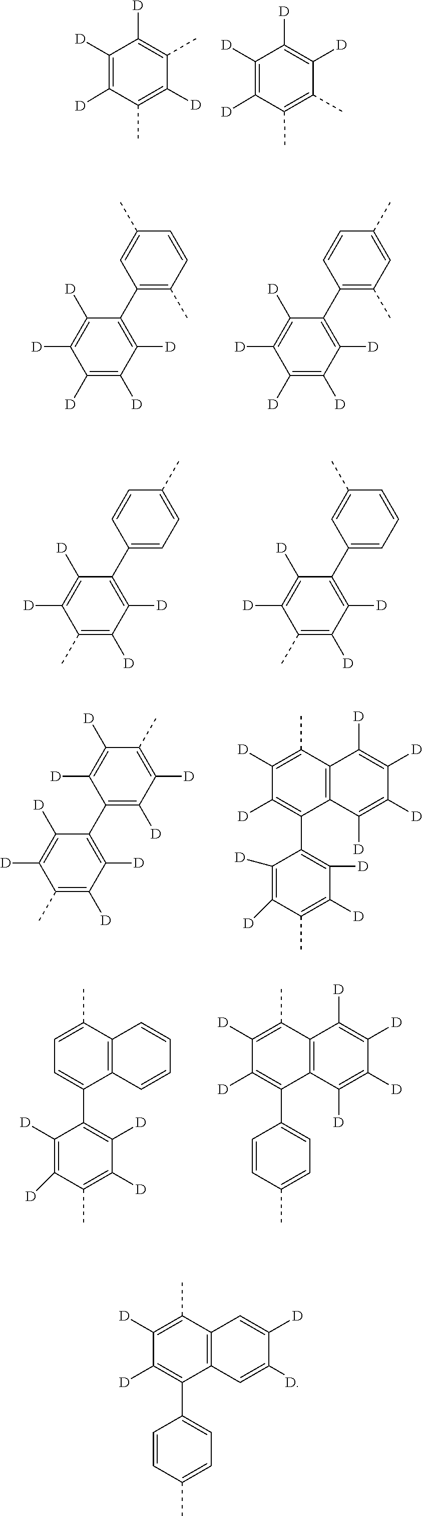

in each of Chemical Formulas, the dotted line represents a bonding position.

Further, in Chemical Formula 1, a represents the number of R, and when a is 2 or more, two or more Rs may be the same as each other or may be different from each other.

Preferably, a may be 0 or 1.

Further, preferably, R may be hydrogen, deuterium, or a substituted or unsubstituted C₆₋₂₀ aryl, and when R is substituted, it may be substituted with one or more deuterium; a C₁₋₂₀ alkyl such as methyl; or a C₆₋₂₀ aryl such as phenyl or naphthyl.

R may be hydrogen; deuterium; or phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl, which is substituted or unsubstituted.

More preferably, R is hydrogen, deuterium, phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl, each of which, except for hydrogen and deuterium, may be unsubstituted or substituted with deuterium, phenyl or naphthyl.

Preferably, in Chemical Formula 1, R may be deuterium, or at least one of Ar₁, Ar₂, L₁ to L₃ and R may be substituted with deuterium.

Thereby, the compound represented by Chemical Formula 1 may include at least one deuterium substituent. That is, in Chemical Formula 1, n may be an integer of 1 or more.

Preferably, the compound represented by Chemical Formula 1 may contain 1 to 30 deuteriums. In this case, in Chemical Formula 1, n may be an integer of 1 to 30. More preferably, the compound represented by Chemical Formula 1 may include an integer of 1 or more, 2 or more, 3 or more, 4 or more, 5 or more, 6 or more, 8 or more, 10 or more, or 20 or more, and 30 or less, 28 or less, 27 or less, or 25 or less. Thereby, in Chemical Formula 1, n may be an integer of 1 or more, 2 or more, 3 or more, 4 or more, 5 or more, 6 or more, 8 or more, 10 or more, or 20 or more, and 30 or less, 28 or less, 27 or less, or 25 or less.

Representative examples of the compound represented by Chemical Formula 1 are as follows:

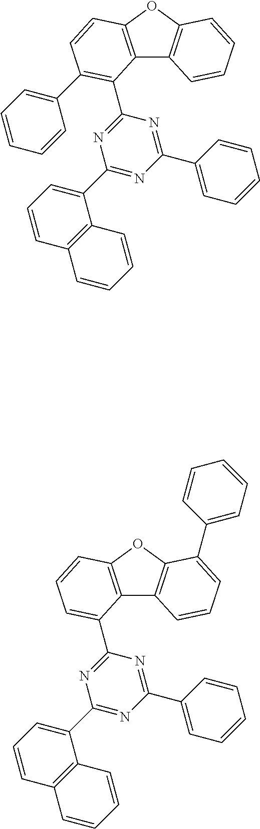 [SMILES: c1ccc(-c2nc(-c3cccc4ccccc34)nc(-c3c(-c4ccccc4)ccc4oc5ccccc5c34)n2)cc1 ; c1ccc(-c2nc(-c3cccc4ccccc34)nc(-c3cccc4oc5c(-c6ccccc6)cccc5c34)n2)cc1]

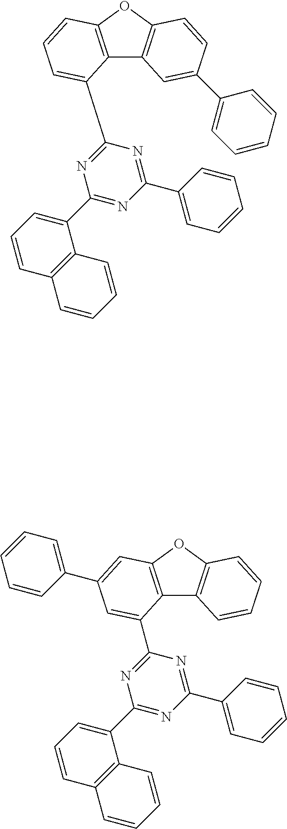 [SMILES: c1ccc(-c2ccc3oc4cccc(-c5nc(-c6ccccc6)nc(-c6cccc7ccccc67)n5)c4c3c2)cc1 ; c1ccc(-c2cc(-c3nc(-c4ccccc4)nc(-c4cccc5ccccc45)n3)c3c(c2)oc2ccccc23)cc1]

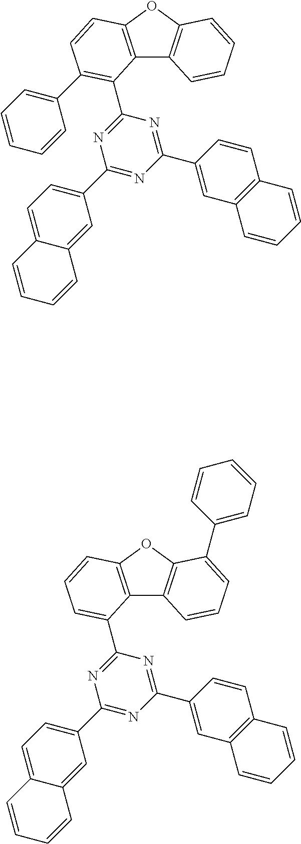 [SMILES: c1ccc(-c2ccc3oc4ccccc4c3c2-c2nc(-c3ccc4ccccc4c3)nc(-c3ccc4ccccc4c3)n2)cc1 ; c1ccc(-c2cccc3c2oc2cccc(-c4nc(-c5ccc6ccccc6c5)nc(-c5ccc6ccccc6c5)n4)c23)cc1]

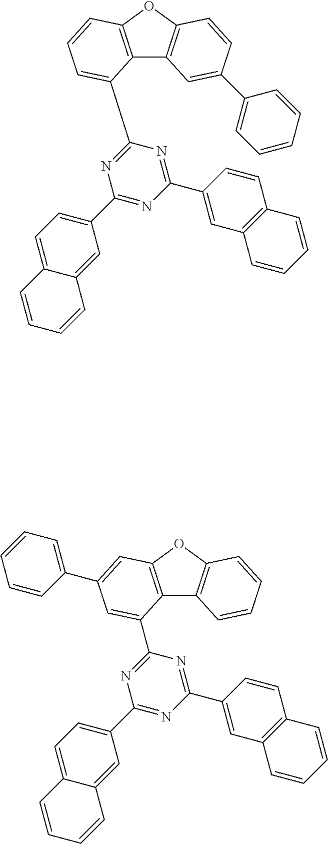 [SMILES: c1ccc(-c2ccc3oc4cccc(-c5nc(-c6ccc7ccccc7c6)nc(-c6ccc7ccccc7c6)n5)c4c3c2)cc1 ; c1ccc(-c2cc(-c3nc(-c4ccc5ccccc5c4)nc(-c4ccc5ccccc5c4)n3)c3c(c2)oc2ccccc23)cc1]

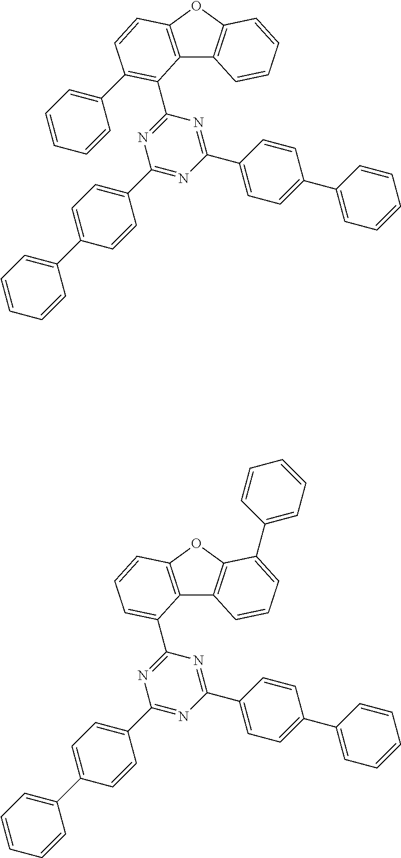 [SMILES: c1ccc(-c2ccc(-c3nc(-c4ccc(-c5ccccc5)cc4)nc(-c4c(-c5ccccc5)ccc5oc6ccccc6c45)n3)cc2)cc1 ; c1ccc(-c2ccc(-c3nc(-c4ccc(-c5ccccc5)cc4)nc(-c4cccc5oc6c(-c7ccccc7)cccc6c45)n3)cc2)cc1]

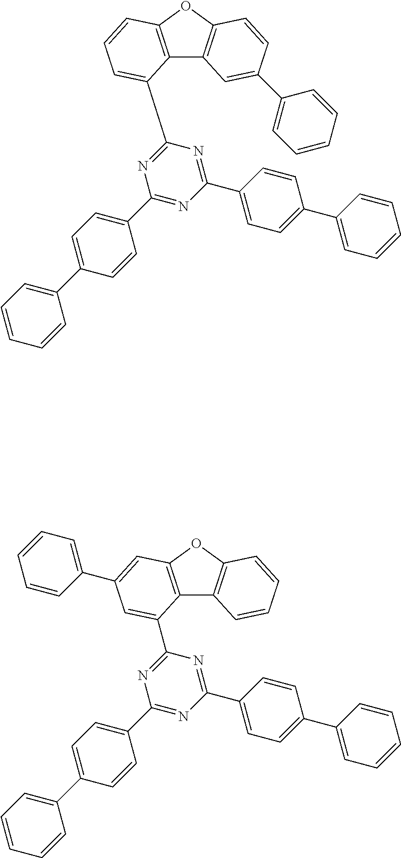 [SMILES: c1ccc(-c2ccc(-c3nc(-c4ccc(-c5ccccc5)cc4)nc(-c4cccc5oc6ccc(-c7ccccc7)cc6c45)n3)cc2)cc1 ; c1ccc(-c2ccc(-c3nc(-c4ccc(-c5ccccc5)cc4)nc(-c4cc(-c5ccccc5)cc5oc6ccccc6c45)n3)cc2)cc1]

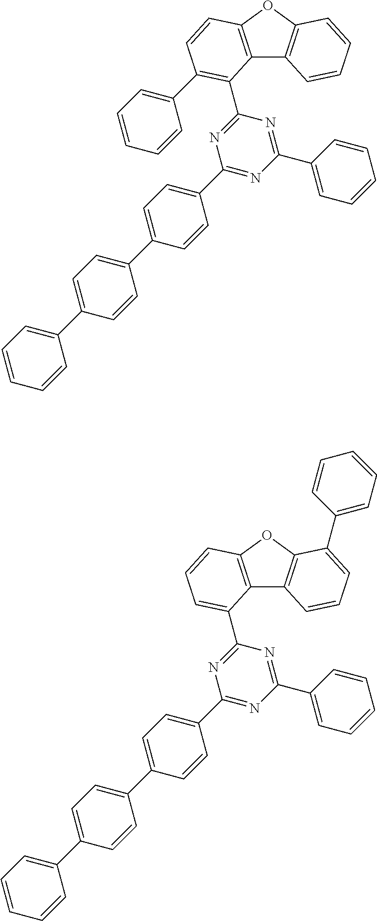 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1]

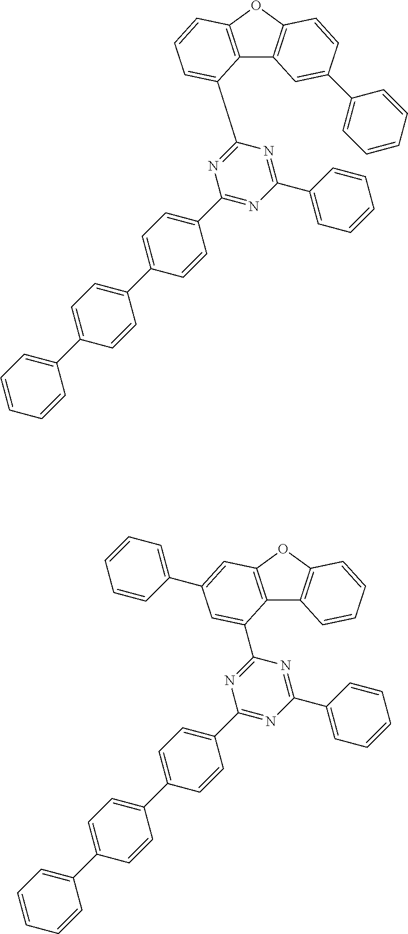 [SMILES: c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1]

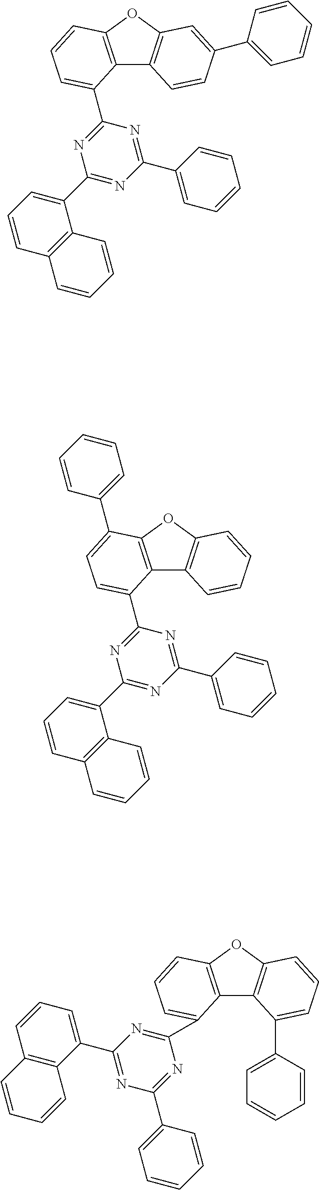 [SMILES: c1ccc(-c2ccc3c(c2)oc2cccc(-c4nc(-c5ccccc5)nc(-c5cccc6ccccc56)n4)c23)cc1 ; c1ccc(-c2nc(-c3cccc4ccccc34)nc(-c3ccc(-c4ccccc4)c4oc5ccccc5c34)n2)cc1 ; c1ccc(-c2nc(-c3cccc4ccccc34)nc(-c3cccc4oc5cccc(-c6ccccc6)c5c34)n2)cc1]

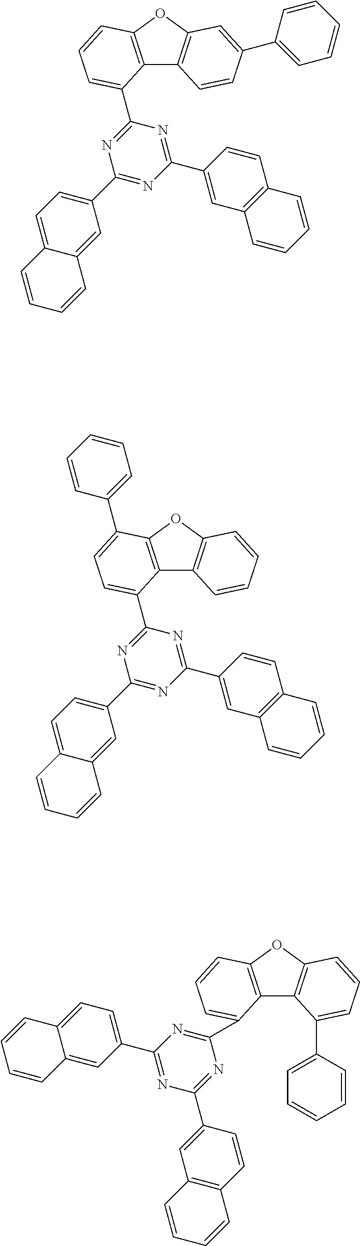 [SMILES: c1ccc(-c2ccc3c(c2)oc2cccc(-c4nc(-c5ccc6ccccc6c5)nc(-c5ccc6ccccc6c5)n4)c23)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2cccc3oc4cccc(-c5nc(-c6ccc7ccccc7c6)nc(-c6ccc7ccccc7c6)n5)c4c23)cc1]

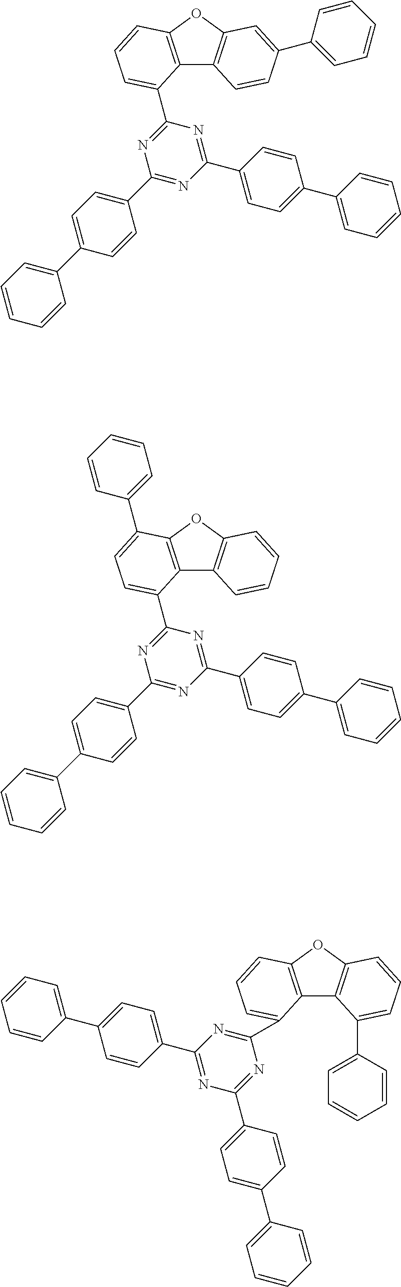 [SMILES: c1ccc(-c2ccc(-c3nc(-c4ccc(-c5ccccc5)cc4)nc(-c4cccc5oc6cc(-c7ccccc7)ccc6c45)n3)cc2)cc1 ; c1ccc(-c2ccc(-c3nc(-c4ccc(-c5ccccc5)cc4)nc(-c4ccc(-c5ccccc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccccc7)c6c45)n3)cc2)cc1]

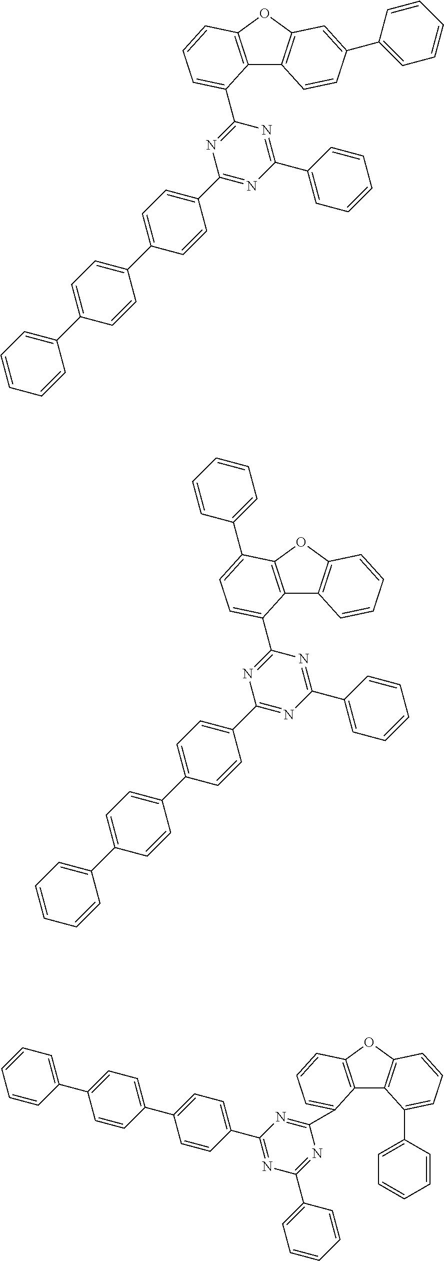 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

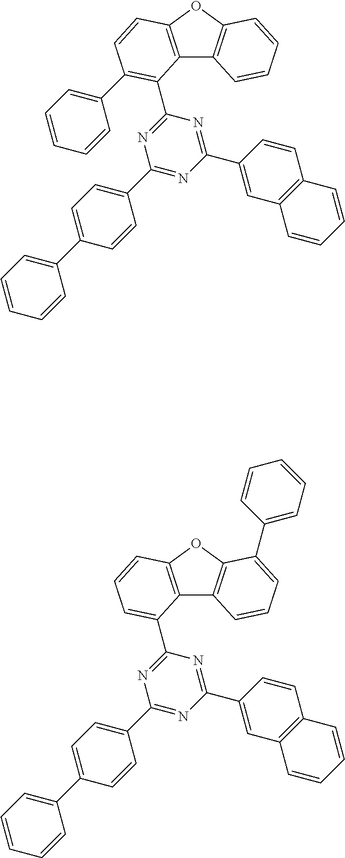 [SMILES: c1ccc(-c2ccc(-c3nc(-c4ccc5ccccc5c4)nc(-c4c(-c5ccccc5)ccc5oc6ccccc6c45)n3)cc2)cc1 ; c1ccc(-c2ccc(-c3nc(-c4ccc5ccccc5c4)nc(-c4cccc5oc6c(-c7ccccc7)cccc6c45)n3)cc2)cc1]

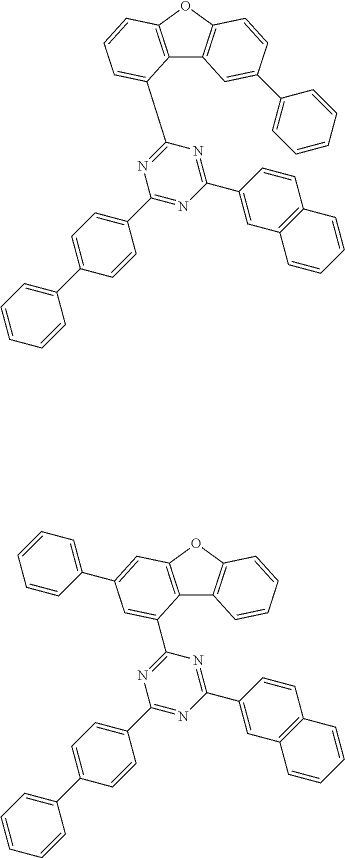 [SMILES: c1ccc(-c2ccc(-c3nc(-c4ccc5ccccc5c4)nc(-c4cccc5oc6ccc(-c7ccccc7)cc6c45)n3)cc2)cc1 ; c1ccc(-c2ccc(-c3nc(-c4ccc5ccccc5c4)nc(-c4cc(-c5ccccc5)cc5oc6ccccc6c45)n3)cc2)cc1]

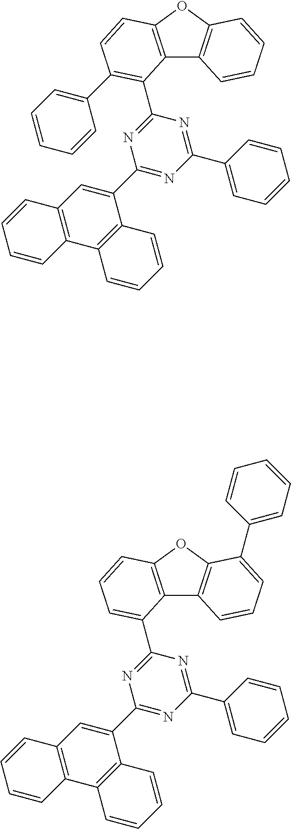 [SMILES: c1ccc(-c2nc(-c3cc4ccccc4c4ccccc34)nc(-c3c(-c4ccccc4)ccc4oc5ccccc5c34)n2)cc1 ; c1ccc(-c2nc(-c3cc4ccccc4c4ccccc34)nc(-c3cccc4oc5c(-c6ccccc6)cccc5c34)n2)cc1]

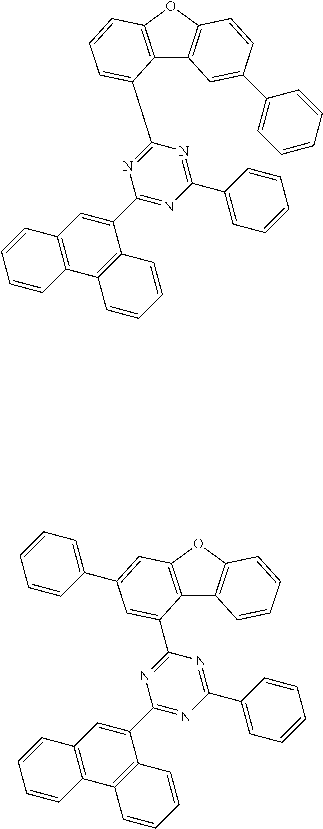 [SMILES: c1ccc(-c2ccc3oc4cccc(-c5nc(-c6ccccc6)nc(-c6cc7ccccc7c7ccccc67)n5)c4c3c2)cc1 ; c1ccc(-c2cc(-c3nc(-c4ccccc4)nc(-c4cc5ccccc5c5ccccc45)n3)c3c(c2)oc2ccccc23)cc1]

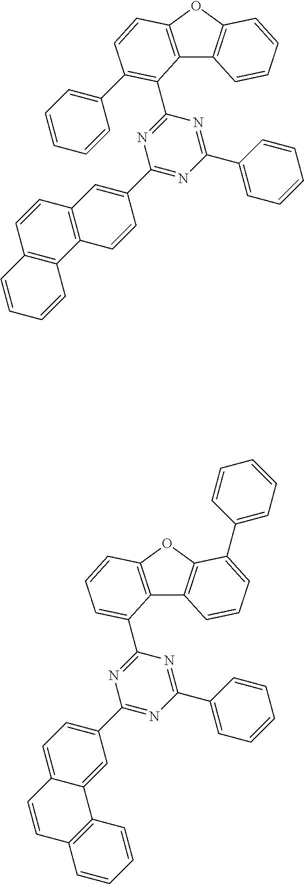 [SMILES: c1ccc(-c2nc(-c3ccc4c(ccc5ccccc54)c3)nc(-c3c(-c4ccccc4)ccc4oc5ccccc5c34)n2)cc1 ; c1ccc(-c2nc(-c3ccc4ccc5ccccc5c4c3)nc(-c3cccc4oc5c(-c6ccccc6)cccc5c34)n2)cc1]

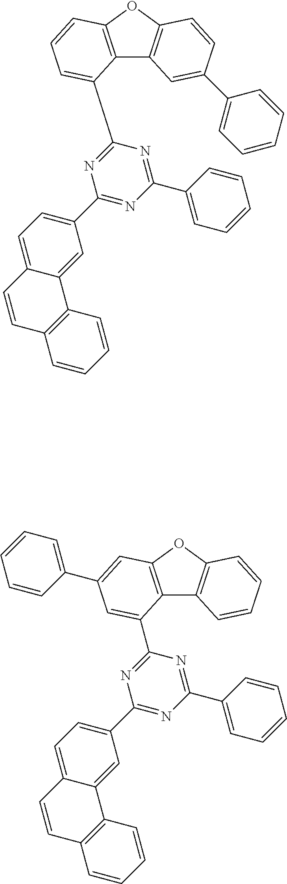 [SMILES: c1ccc(-c2ccc3oc4cccc(-c5nc(-c6ccccc6)nc(-c6ccc7ccc8ccccc8c7c6)n5)c4c3c2)cc1 ; c1ccc(-c2cc(-c3nc(-c4ccccc4)nc(-c4ccc5ccc6ccccc6c5c4)n3)c3c(c2)oc2ccccc23)cc1]

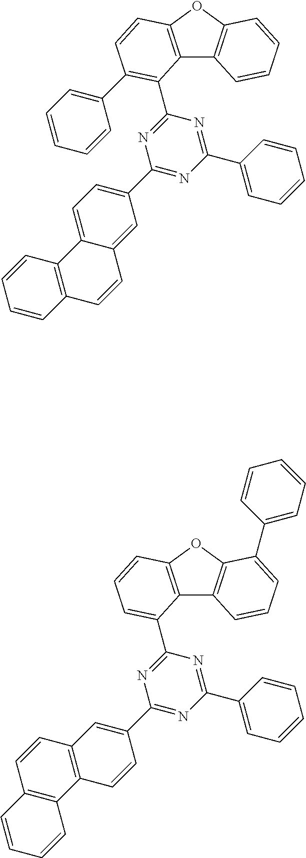 [SMILES: c1ccc(-c2nc(-c3ccc4c(ccc5ccccc54)c3)nc(-c3c(-c4ccccc4)ccc4oc5ccccc5c34)n2)cc1 ; c1ccc(-c2nc(-c3ccc4c(ccc5ccccc54)c3)nc(-c3cccc4oc5c(-c6ccccc6)cccc5c34)n2)cc1]

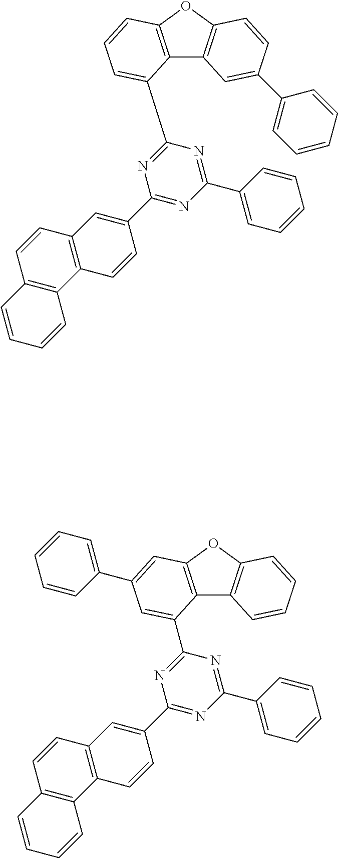 [SMILES: c1ccc(-c2ccc3oc4cccc(-c5nc(-c6ccccc6)nc(-c6ccc7c(ccc8ccccc87)c6)n5)c4c3c2)cc1 ; c1ccc(-c2cc(-c3nc(-c4ccccc4)nc(-c4ccc5c(ccc6ccccc65)c4)n3)c3c(c2)oc2ccccc23)cc1]

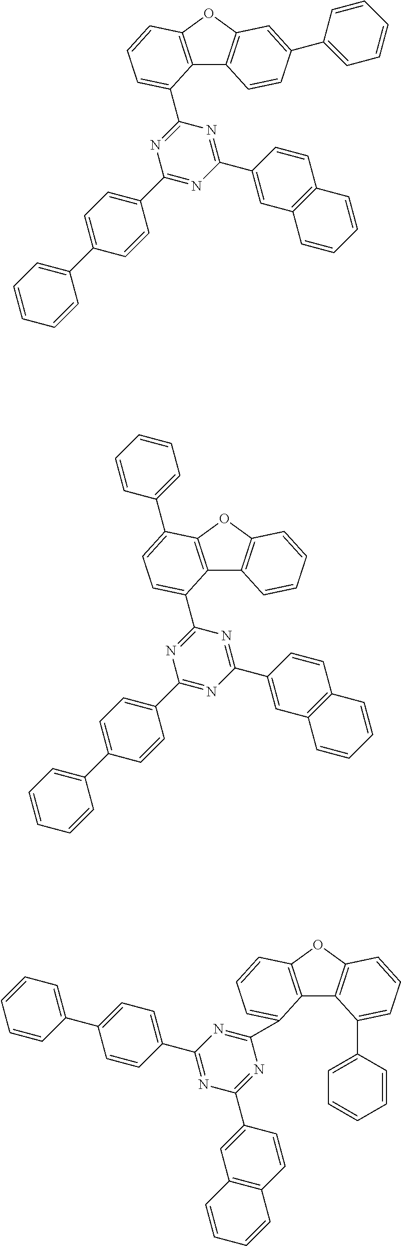 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1]

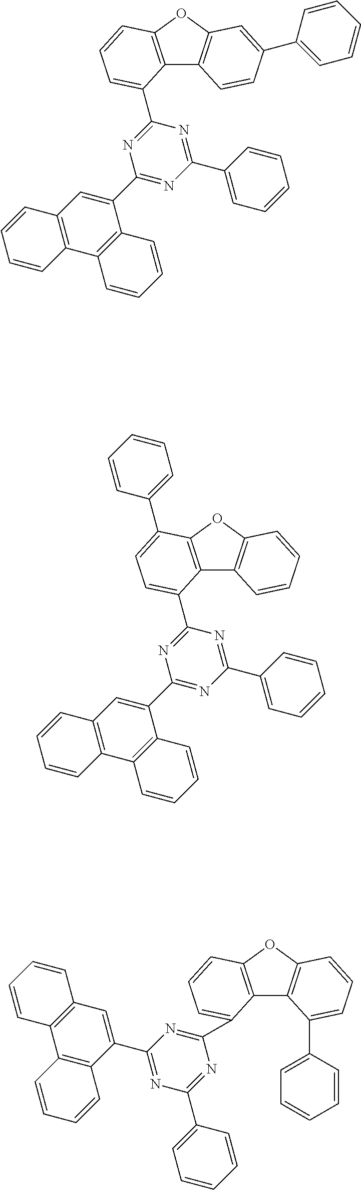 [SMILES: c1ccc(-c2ccc3c(c2)oc2cccc(-c4nc(-c5ccccc5)nc(-c5cc6ccccc6c6ccccc56)n4)c23)cc1 ; c1ccc(-c2nc(-c3cc4ccccc4c4ccccc34)nc(-c3ccc(-c4ccccc4)c4oc5ccccc5c34)n2)cc1 ; c1ccc(-c2nc(-c3cc4ccccc4c4ccccc34)nc(-c3cccc4oc5cccc(-c6ccccc6)c5c34)n2)cc1]

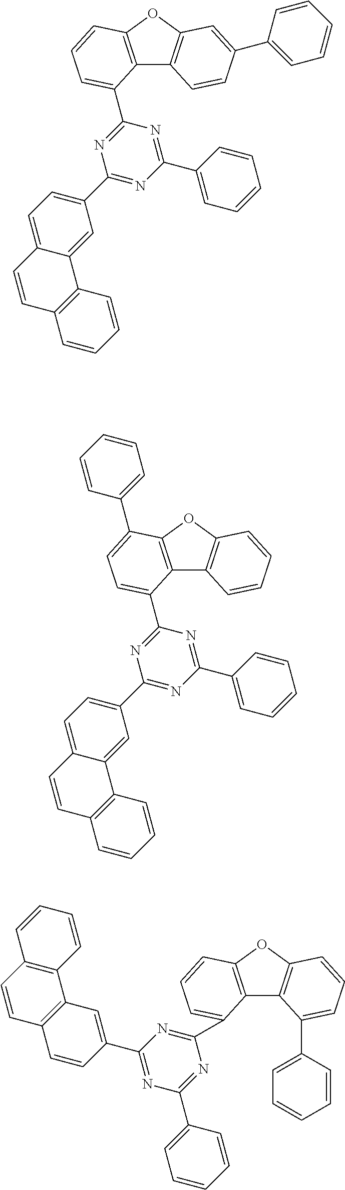 [SMILES: c1ccc(-c2ccc3c(c2)oc2cccc(-c4nc(-c5ccccc5)nc(-c5ccc6ccc7ccccc7c6c5)n4)c23)cc1 ; c1ccc(-c2nc(-c3ccc4ccc5ccccc5c4c3)nc(-c3ccc(-c4ccccc4)c4oc5ccccc5c34)n2)cc1 ; c1ccc(-c2nc(-c3ccc4ccc5ccccc5c4c3)nc(-c3cccc4oc5cccc(-c6ccccc6)c5c34)n2)cc1]

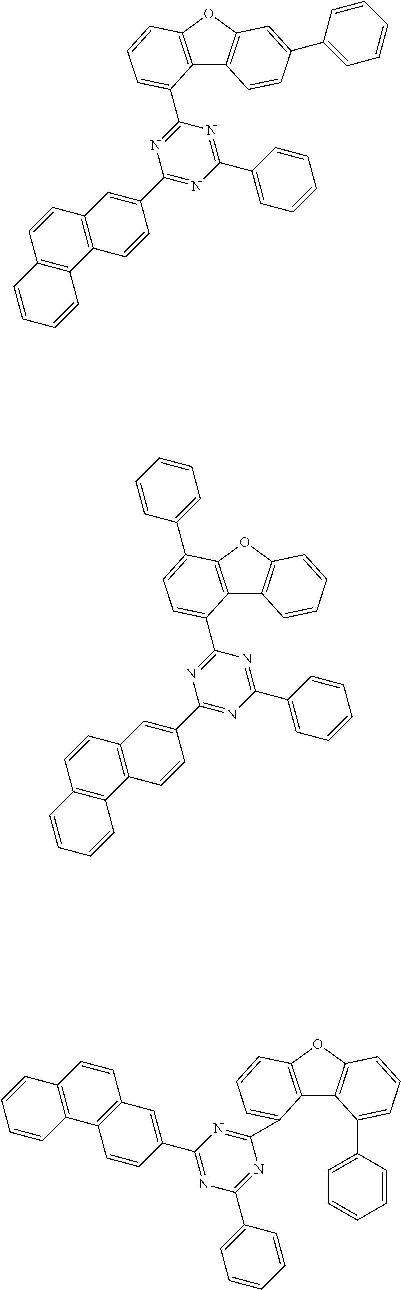 [SMILES: c1ccc(-c2ccc3c(c2)oc2cccc(-c4nc(-c5ccccc5)nc(-c5ccc6c(ccc7ccccc76)c5)n4)c23)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

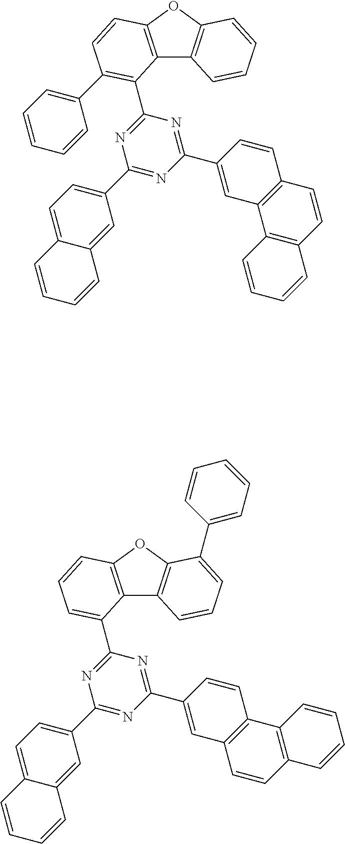 [SMILES: c1ccc(-c2ccc3oc4ccccc4c3c2-c2nc(-c3ccc4ccccc4c3)nc(-c3ccc4ccc5ccccc5c4c3)n2)cc1 ; c1ccc(-c2cccc3c2oc2cccc(-c4nc(-c5ccc6ccccc6c5)nc(-c5ccc6c(ccc7ccccc76)c5)n4)c23)cc1]

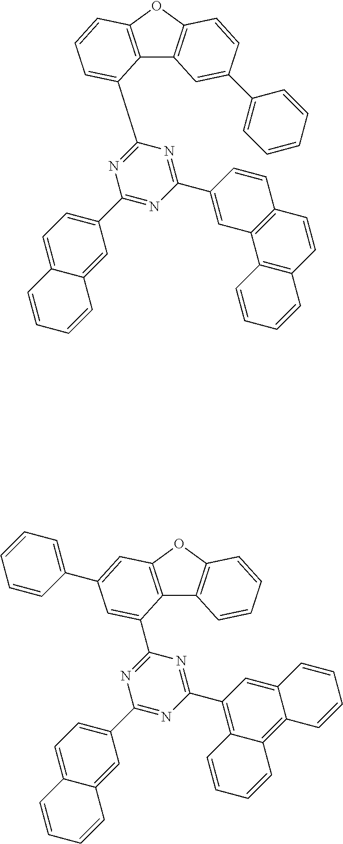 [SMILES: c1ccc(-c2ccc3oc4cccc(-c5nc(-c6ccc7ccccc7c6)nc(-c6ccc7ccc8ccccc8c7c6)n5)c4c3c2)cc1 ; c1ccc(-c2cc(-c3nc(-c4ccc5ccccc5c4)nc(-c4cc5ccccc5c5ccccc45)n3)c3c(c2)oc2ccccc23)cc1]

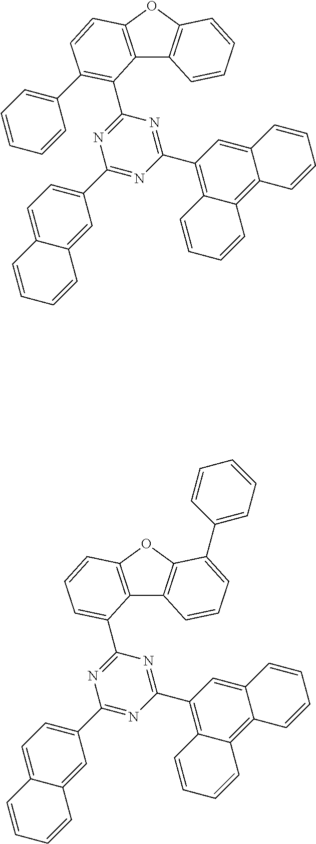 [SMILES: c1ccc(-c2ccc3oc4ccccc4c3c2-c2nc(-c3ccc4ccccc4c3)nc(-c3cc4ccccc4c4ccccc34)n2)cc1 ; c1ccc(-c2cccc3c2oc2cccc(-c4nc(-c5ccc6ccccc6c5)nc(-c5cc6ccccc6c6ccccc56)n4)c23)cc1]

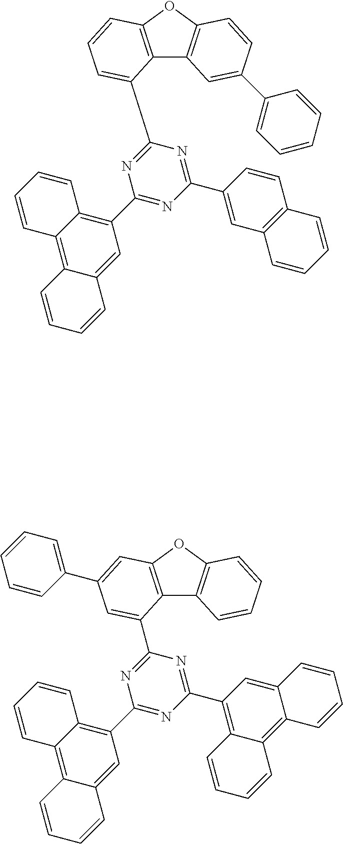 [SMILES: c1ccc(-c2ccc3oc4cccc(-c5nc(-c6ccc7ccccc7c6)nc(-c6cc7ccccc7c7ccccc67)n5)c4c3c2)cc1 ; c1ccc(-c2cc(-c3nc(-c4cc5ccccc5c5ccccc45)nc(-c4cc5ccccc5c5ccccc45)n3)c3c(c2)oc2ccccc23)cc1]

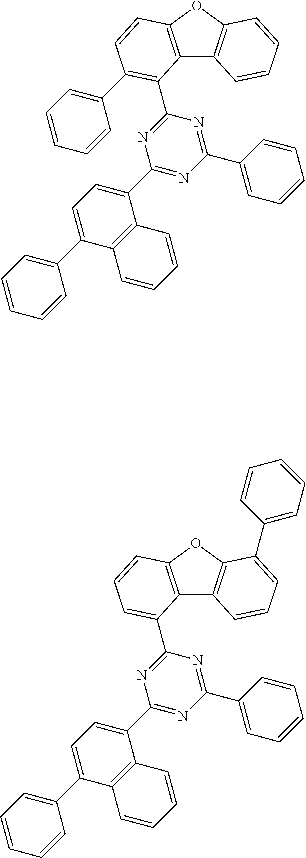 [SMILES: c1ccc(-c2nc(-c3ccc(-c4ccccc4)c4ccccc34)nc(-c3c(-c4ccccc4)ccc4oc5ccccc5c34)n2)cc1 ; c1ccc(-c2nc(-c3ccc(-c4ccccc4)c4ccccc34)nc(-c3cccc4oc5c(-c6ccccc6)cccc5c34)n2)cc1]

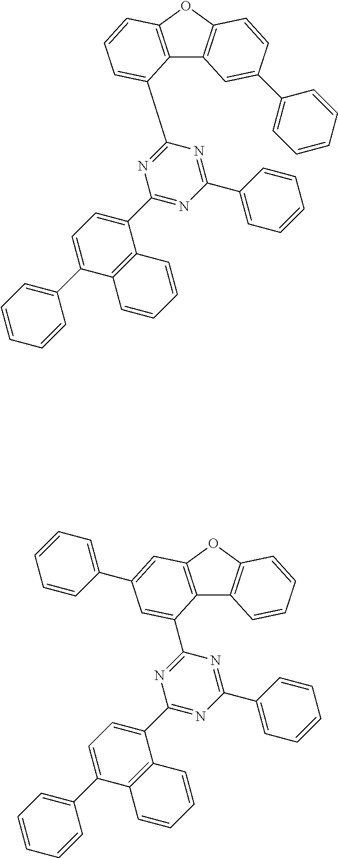 [SMILES: c1ccc(-c2ccc3oc4cccc(-c5nc(-c6ccccc6)nc(-c6ccc(-c7ccccc7)c7ccccc67)n5)c4c3c2)cc1 ; c1ccc(-c2cc(-c3nc(-c4ccccc4)nc(-c4ccc(-c5ccccc5)c5ccccc45)n3)c3c(c2)oc2ccccc23)cc1]

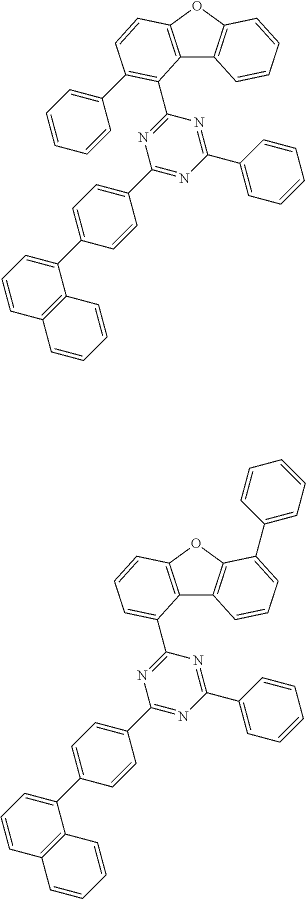 [SMILES: c1ccc(-c2nc(-c3ccc(-c4cccc5ccccc45)cc3)nc(-c3c(-c4ccccc4)ccc4oc5ccccc5c34)n2)cc1 ; c1ccc(-c2nc(-c3ccc(-c4cccc5ccccc45)cc3)nc(-c3cccc4oc5c(-c6ccccc6)cccc5c34)n2)cc1]

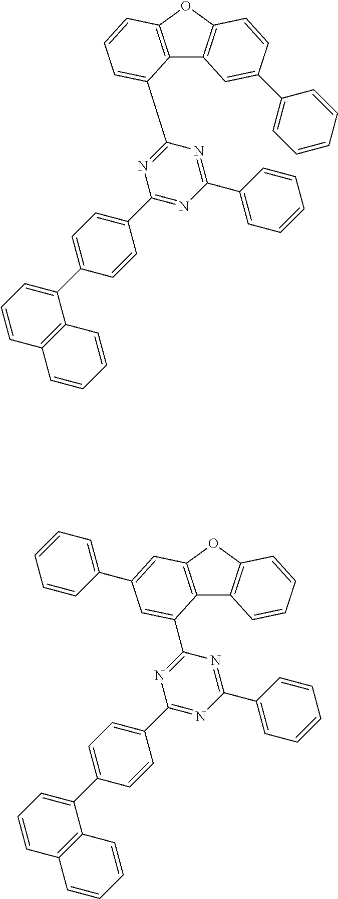 [SMILES: c1ccc(-c2ccc3oc4cccc(-c5nc(-c6ccccc6)nc(-c6ccc(-c7cccc8ccccc78)cc6)n5)c4c3c2)cc1 ; c1ccc(-c2cc(-c3nc(-c4ccccc4)nc(-c4ccc(-c5cccc6ccccc56)cc4)n3)c3c(c2)oc2ccccc23)cc1]

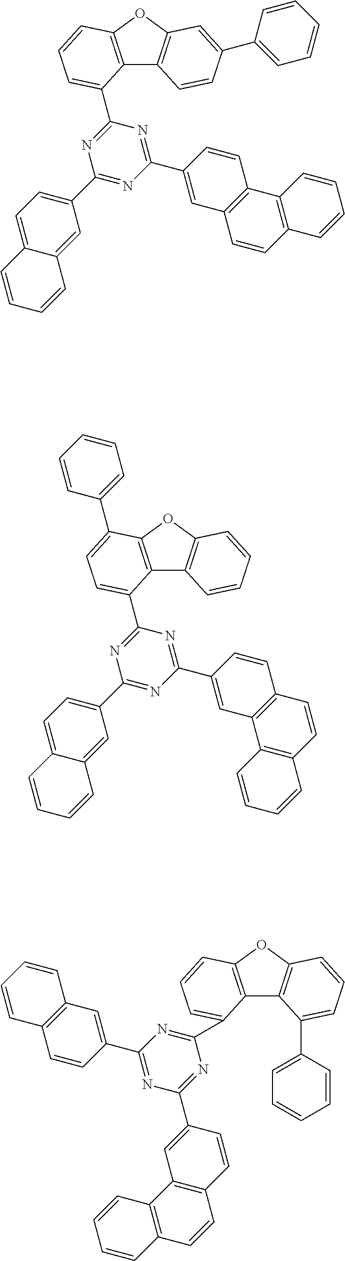 [SMILES: c1ccc(-c2ccc3c(c2)oc2cccc(-c4nc(-c5ccc6ccccc6c5)nc(-c5ccc6c(ccc7ccccc76)c5)n4)c23)cc1 ; c1ccc(-c2ccc(-c3nc(-c4ccc5ccccc5c4)nc(-c4ccc5ccc6ccccc6c5c4)n3)c3c2oc2ccccc23)cc1 ; c1ccc(-c2cccc3oc4cccc(-c5nc(-c6ccc7ccccc7c6)nc(-c6ccc7ccc8ccccc8c7c6)n5)c4c23)cc1]

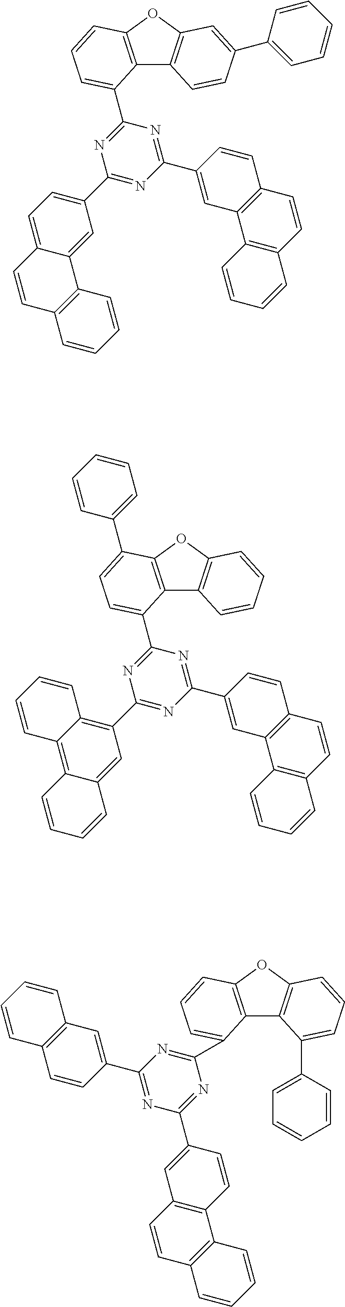 [SMILES: c1ccc(-c2ccc3c(c2)oc2cccc(-c4nc(-c5ccc6ccc7ccccc7c6c5)nc(-c5ccc6ccc7ccccc7c6c5)n4)c23)cc1 ; c1ccc(-c2ccc(-c3nc(-c4ccc5ccc6ccccc6c5c4)nc(-c4cc5ccccc5c5ccccc45)n3)c3c2oc2ccccc23)cc1 ; c1ccc(-c2cccc3oc4cccc(-c5nc(-c6ccc7ccccc7c6)nc(-c6ccc7c(ccc8ccccc87)c6)n5)c4c23)cc1]

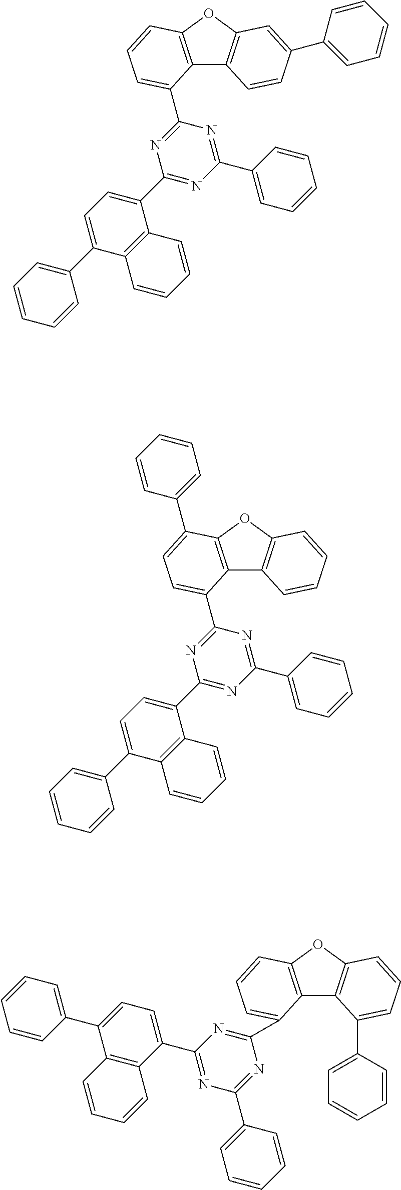 [SMILES: c1ccc(-c2ccc3c(c2)oc2cccc(-c4nc(-c5ccccc5)nc(-c5ccc(-c6ccccc6)c6ccccc56)n4)c23)cc1 ; c1ccc(-c2nc(-c3ccc(-c4ccccc4)c4ccccc34)nc(-c3ccc(-c4ccccc4)c4oc5ccccc5c34)n2)cc1 ; c1ccc(-c2nc(-c3ccc(-c4ccccc4)c4ccccc34)nc(-c3cccc4oc5cccc(-c6ccccc6)c5c34)n2)cc1]

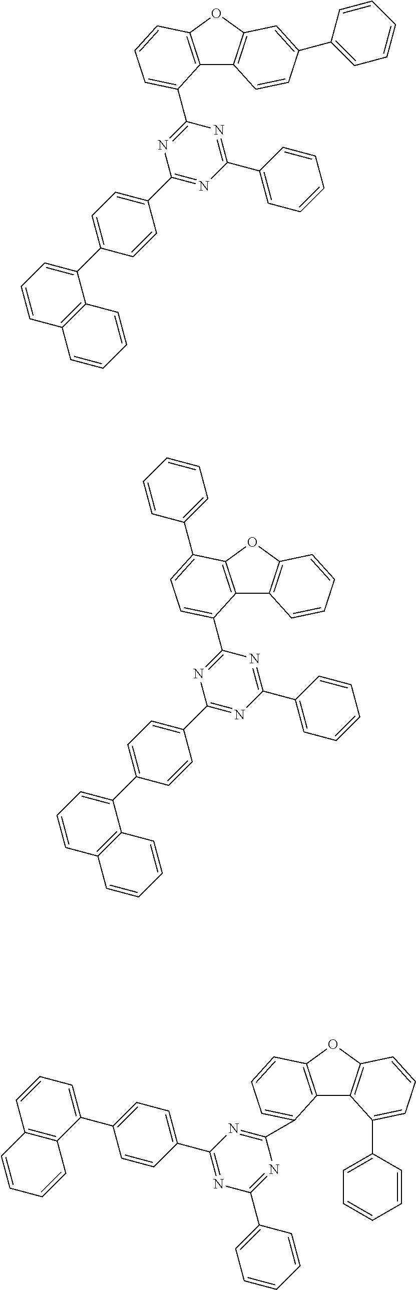 [SMILES: c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3ccc(-c4ccccc4)c4oc5ccccc5c34)n2)cc1 ; c1ccc(-c2nc(-c3ccc(-c4cccc5ccccc45)cc3)nc(-c3cccc4oc5cccc(-c6ccccc6)c5c34)n2)cc1]

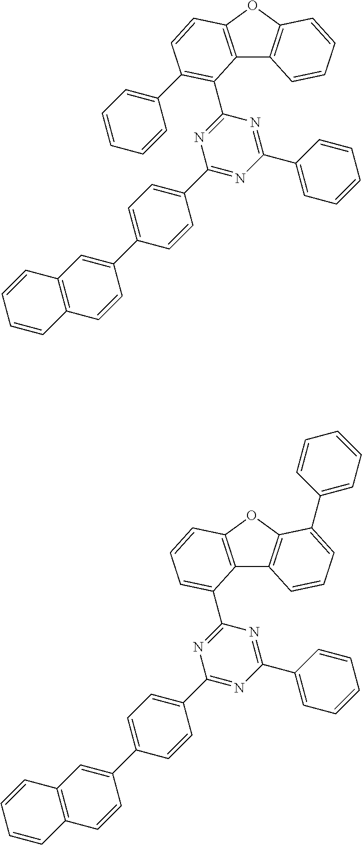 [SMILES: c1ccc(-c2nc(-c3ccc(-c4ccc5ccccc5c4)cc3)nc(-c3c(-c4ccccc4)ccc4oc5ccccc5c34)n2)cc1 ; c1ccc(-c2nc(-c3ccc(-c4ccc5ccccc5c4)cc3)nc(-c3cccc4oc5c(-c6ccccc6)cccc5c34)n2)cc1]

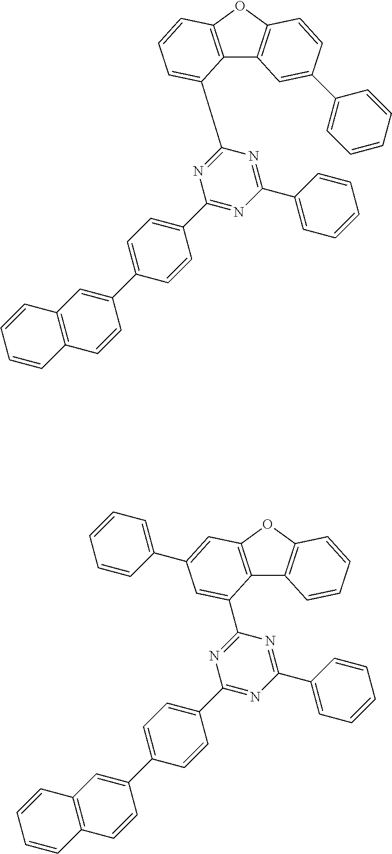 [SMILES: c1ccc(-c2ccc3oc4cccc(-c5nc(-c6ccccc6)nc(-c6ccc(-c7ccc8ccccc8c7)cc6)n5)c4c3c2)cc1 ; c1ccc(-c2cc(-c3nc(-c4ccccc4)nc(-c4ccc(-c5ccc6ccccc6c5)cc4)n3)c3c(c2)oc2ccccc23)cc1]

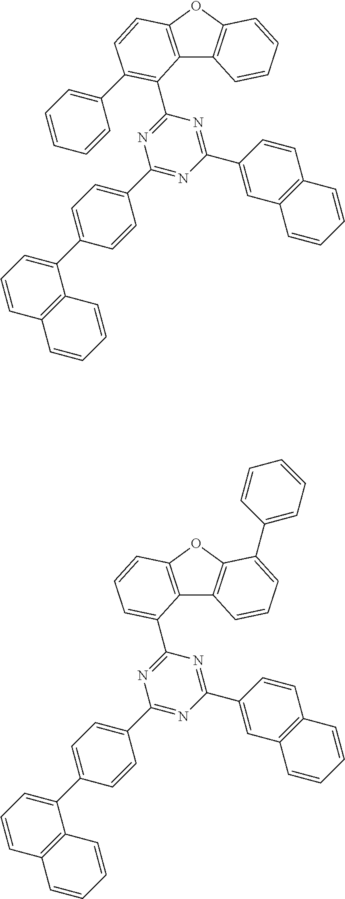 [SMILES: c1ccc(-c2ccc3oc4ccccc4c3c2-c2nc(-c3ccc(-c4cccc5ccccc45)cc3)nc(-c3ccc4ccccc4c3)n2)cc1 ; c1ccc(-c2cccc3c2oc2cccc(-c4nc(-c5ccc(-c6cccc7ccccc67)cc5)nc(-c5ccc6ccccc6c5)n4)c23)cc1]

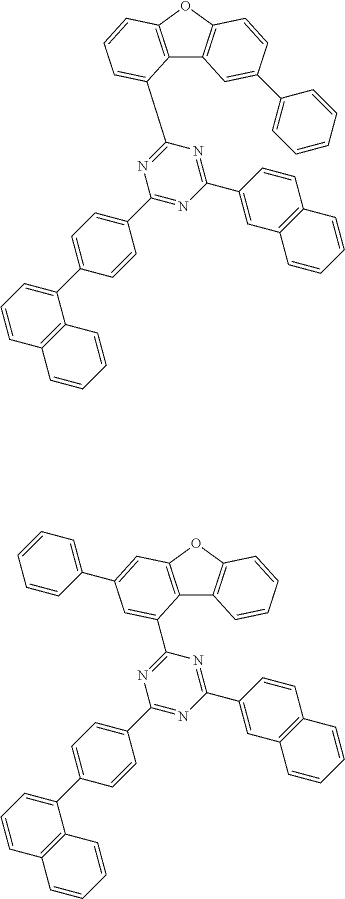 [SMILES: c1ccc(-c2ccc3oc4cccc(-c5nc(-c6ccc(-c7cccc8ccccc78)cc6)nc(-c6ccc7ccccc7c6)n5)c4c3c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1]

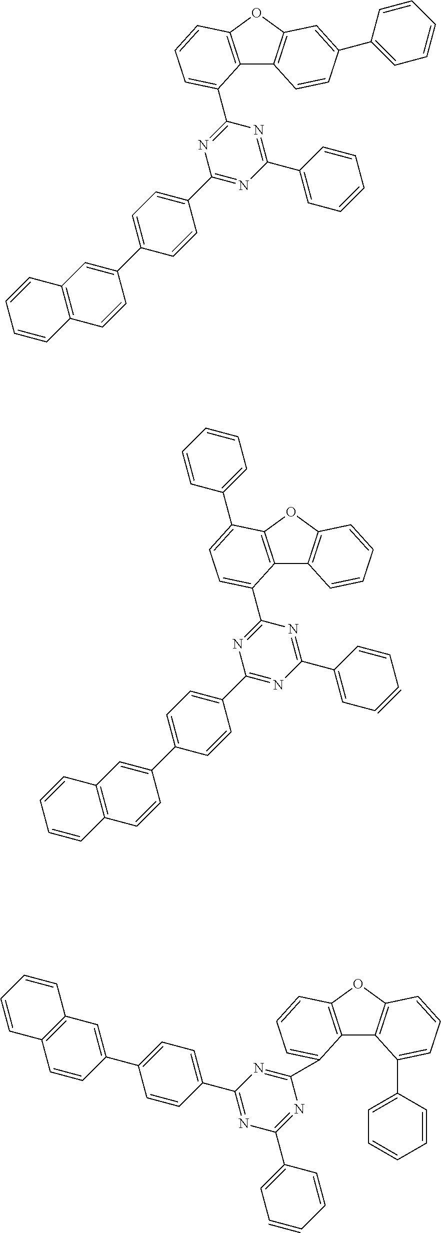 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2nc(-c3ccc(-c4ccc5ccccc5c4)cc3)nc(-c3ccc(-c4ccccc4)c4oc5ccccc5c34)n2)cc1 ; c1ccc(-c2nc(-c3ccc(-c4ccc5ccccc5c4)cc3)nc(-c3cccc4oc5cccc(-c6ccccc6)c5c34)n2)cc1]

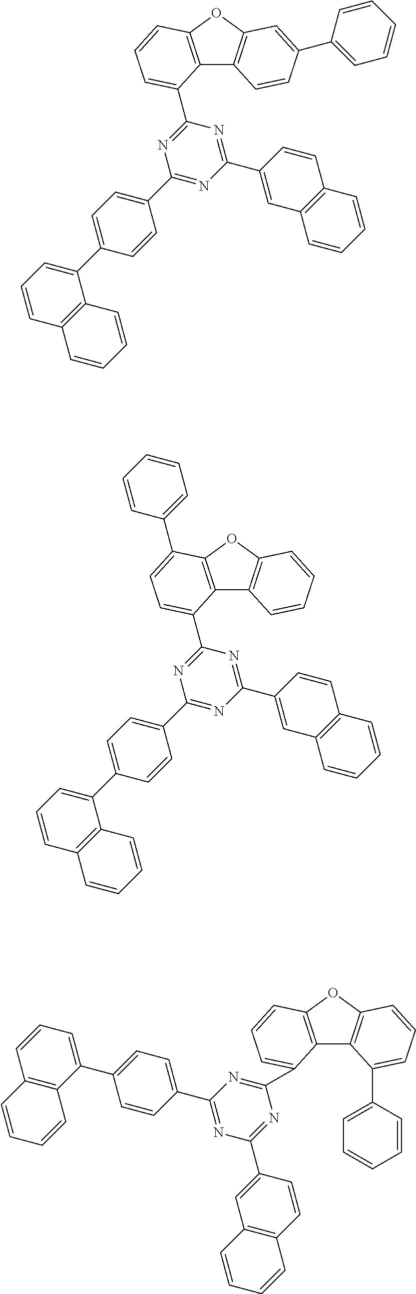 [SMILES: c1ccc(-c2ccc3c(c2)oc2cccc(-c4nc(-c5ccc(-c6cccc7ccccc67)cc5)nc(-c5ccc6ccccc6c5)n4)c23)cc1 ; c1ccc(-c2ccc(-c3nc(-c4ccc(-c5cccc6ccccc56)cc4)nc(-c4ccc5ccccc5c4)n3)c3c2oc2ccccc23)cc1 ; c1ccc(-c2cccc3oc4cccc(-c5nc(-c6ccc(-c7cccc8ccccc78)cc6)nc(-c6ccc7ccccc7c6)n5)c4c23)cc1]

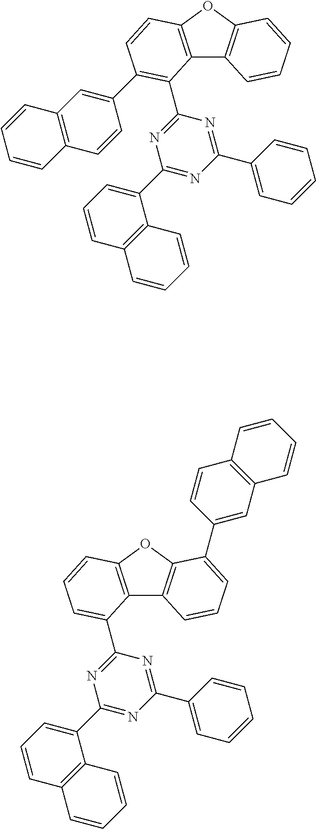 [SMILES: c1ccc(-c2nc(-c3cccc4ccccc34)nc(-c3c(-c4ccc5ccccc5c4)ccc4oc5ccccc5c34)n2)cc1 ; c1ccc(-c2nc(-c3cccc4ccccc34)nc(-c3cccc4oc5c(-c6ccc7ccccc7c6)cccc5c34)n2)cc1]

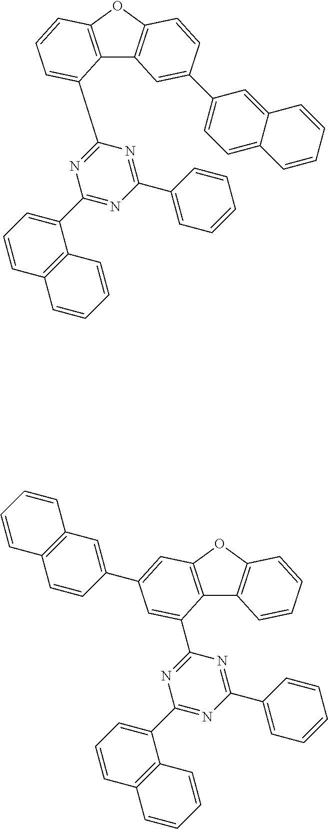 [SMILES: c1ccc(-c2nc(-c3cccc4ccccc34)nc(-c3cccc4oc5ccc(-c6ccc7ccccc7c6)cc5c34)n2)cc1 ; c1ccc(-c2nc(-c3cccc4ccccc34)nc(-c3cc(-c4ccc5ccccc5c4)cc4oc5ccccc5c34)n2)cc1]

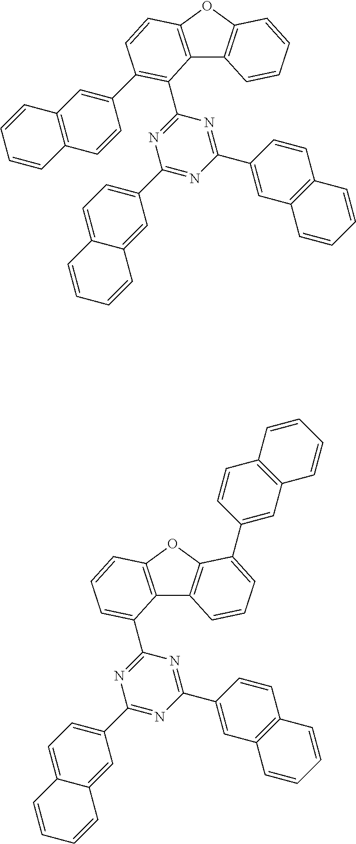 [SMILES: c1ccc2cc(-c3nc(-c4ccc5ccccc5c4)nc(-c4c(-c5ccc6ccccc6c5)ccc5oc6ccccc6c45)n3)ccc2c1 ; c1ccc2cc(-c3nc(-c4ccc5ccccc5c4)nc(-c4cccc5oc6c(-c7ccc8ccccc8c7)cccc6c45)n3)ccc2c1]

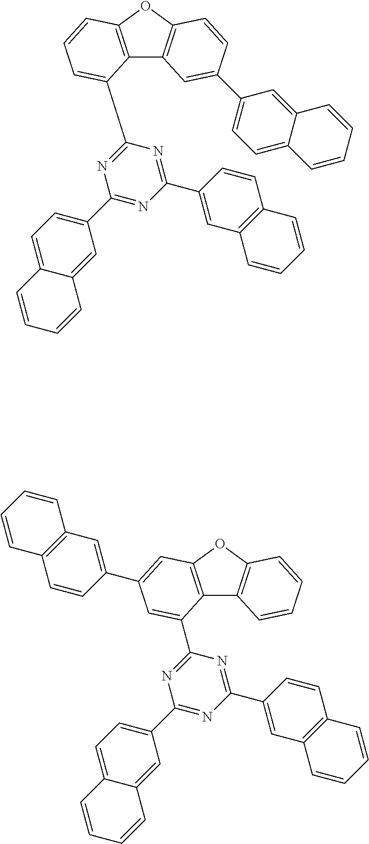 [SMILES: c1ccc2cc(-c3ccc4oc5cccc(-c6nc(-c7ccc8ccccc8c7)nc(-c7ccc8ccccc8c7)n6)c5c4c3)ccc2c1 ; c1ccc2cc(-c3cc(-c4nc(-c5ccc6ccccc6c5)nc(-c5ccc6ccccc6c5)n4)c4c(c3)oc3ccccc34)ccc2c1]

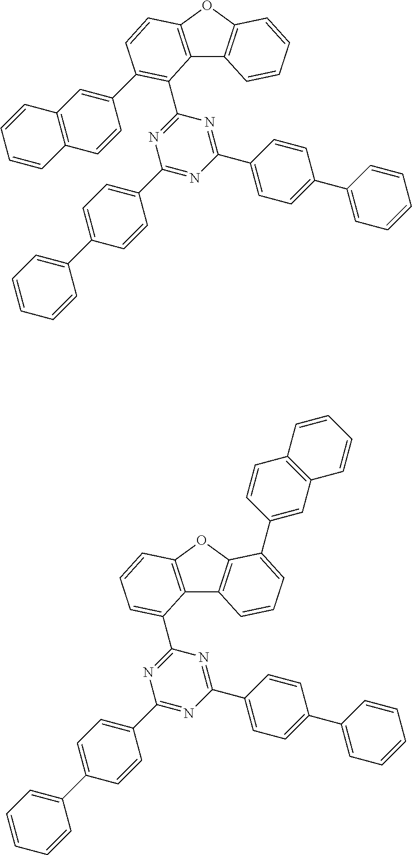 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1]

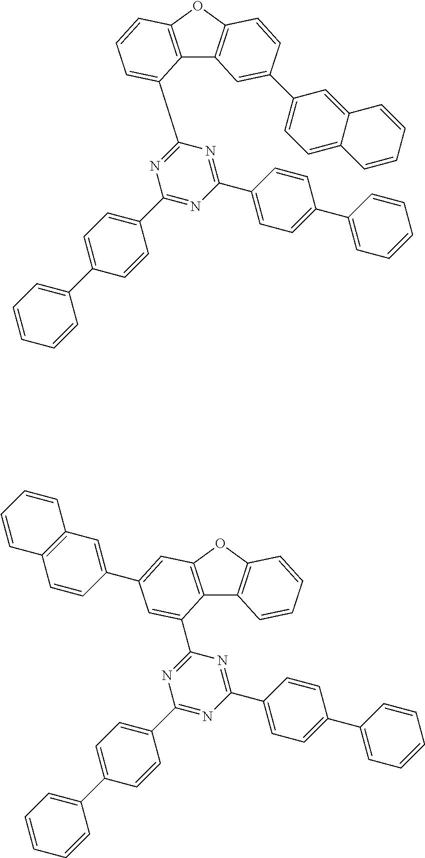 [SMILES: c1ccc(-c2ccc(-c3nc(-c4ccc(-c5ccccc5)cc4)nc(-c4cccc5oc6ccc(-c7ccc8ccccc8c7)cc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1]

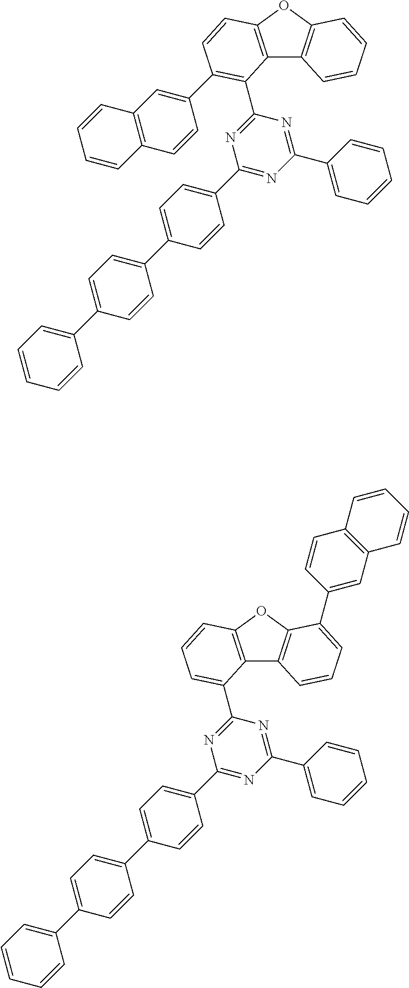 [SMILES: c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

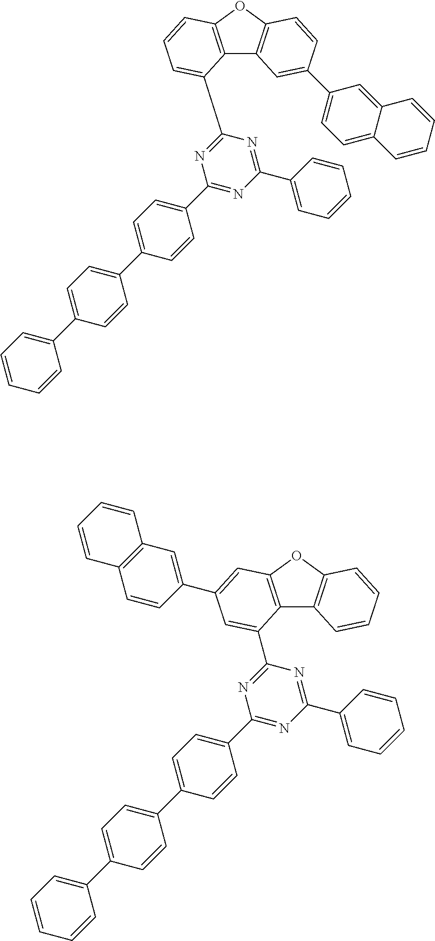 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

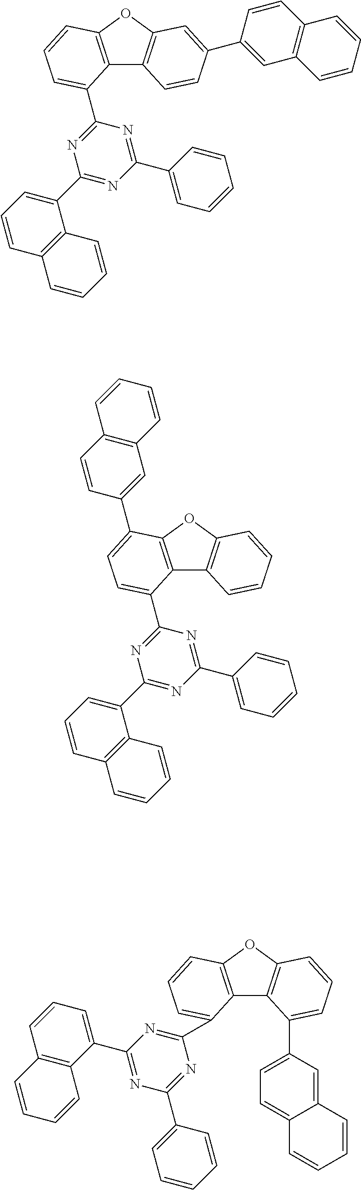 [SMILES: c1ccc(-c2nc(-c3cccc4ccccc34)nc(-c3cccc4oc5cc(-c6ccc7ccccc7c6)ccc5c34)n2)cc1 ; c1ccc(-c2nc(-c3cccc4ccccc34)nc(-c3ccc(-c4ccc5ccccc5c4)c4oc5ccccc5c34)n2)cc1 ; c1ccc(-c2nc(-c3cccc4ccccc34)nc(-c3cccc4oc5cccc(-c6ccc7ccccc7c6)c5c34)n2)cc1]

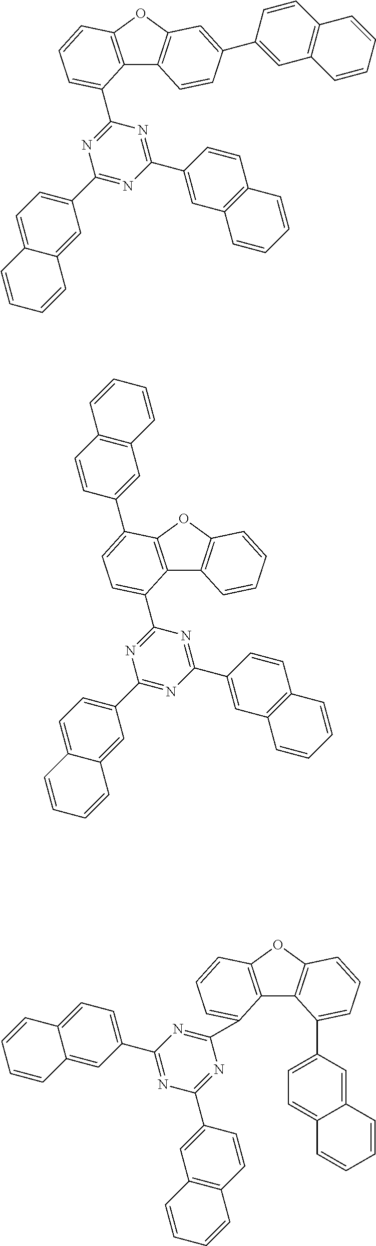 [SMILES: c1ccc2cc(-c3ccc4c(c3)oc3cccc(-c5nc(-c6ccc7ccccc7c6)nc(-c6ccc7ccccc7c6)n5)c34)ccc2c1 ; c1ccc2cc(-c3nc(-c4ccc5ccccc5c4)nc(-c4ccc(-c5ccc6ccccc6c5)c5oc6ccccc6c45)n3)ccc2c1 ; c1ccc2cc(-c3nc(-c4ccc5ccccc5c4)nc(-c4cccc5oc6cccc(-c7ccc8ccccc8c7)c6c45)n3)ccc2c1]

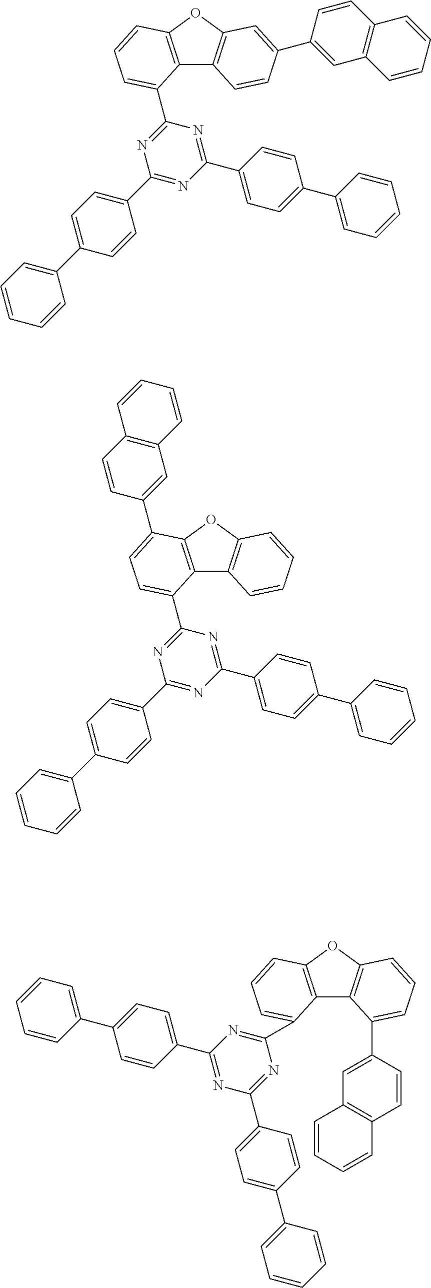 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1]

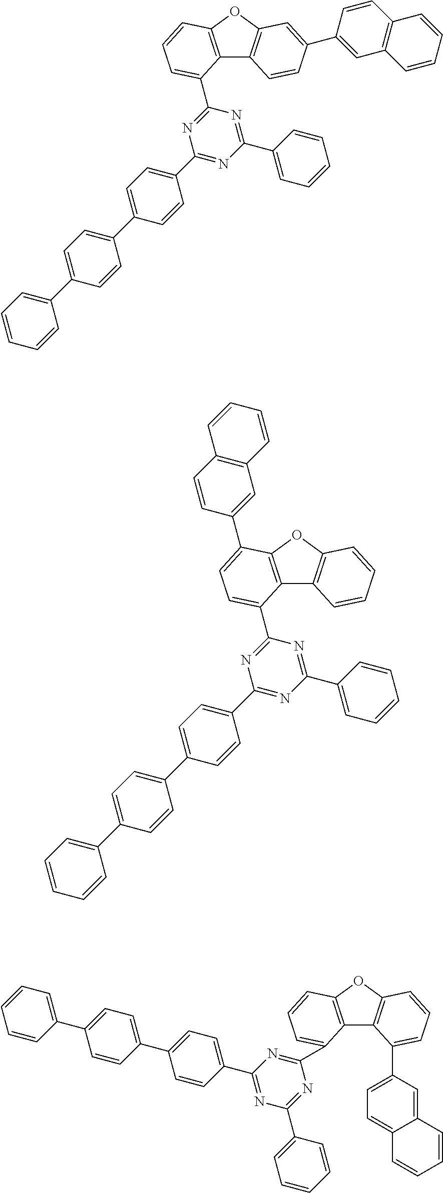 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

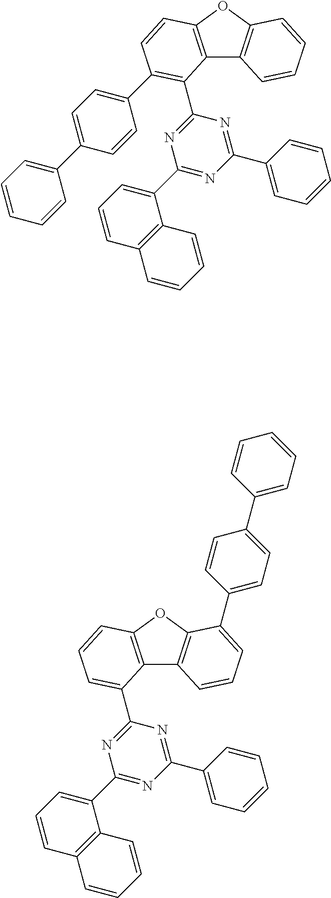 [SMILES: c1ccc(-c2ccc(-c3ccc4oc5ccccc5c4c3-c3nc(-c4ccccc4)nc(-c4cccc5ccccc45)n3)cc2)cc1 ; c1ccc(-c2ccc(-c3cccc4c3oc3cccc(-c5nc(-c6ccccc6)nc(-c6cccc7ccccc67)n5)c34)cc2)cc1]

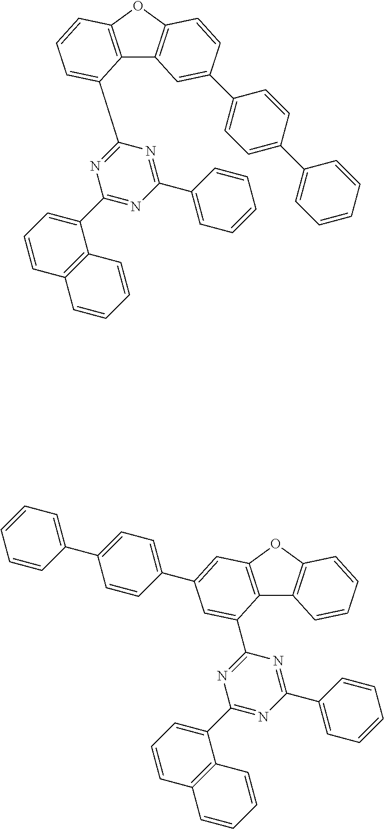 [SMILES: c1ccc(-c2ccc(-c3ccc4oc5cccc(-c6nc(-c7ccccc7)nc(-c7cccc8ccccc78)n6)c5c4c3)cc2)cc1 ; c1ccc(-c2ccc(-c3cc(-c4nc(-c5ccccc5)nc(-c5cccc6ccccc56)n4)c4c(c3)oc3ccccc34)cc2)cc1]

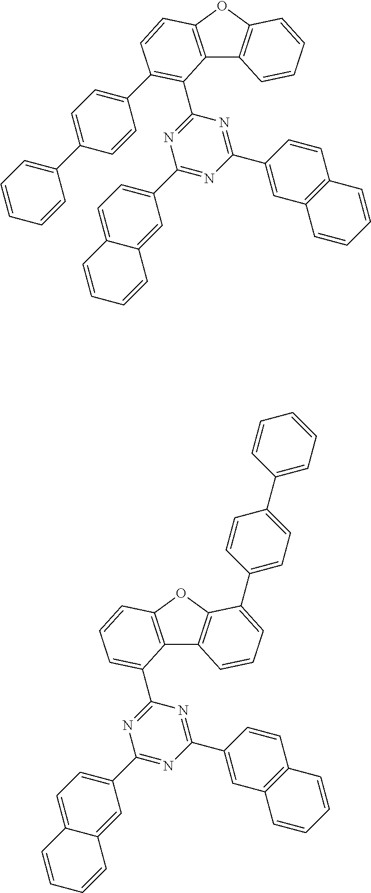 [SMILES: c1ccc(-c2ccc(-c3ccc4oc5ccccc5c4c3-c3nc(-c4ccc5ccccc5c4)nc(-c4ccc5ccccc5c4)n3)cc2)cc1 ; c1ccc(-c2ccc(-c3cccc4c3oc3cccc(-c5nc(-c6ccc7ccccc7c6)nc(-c6ccc7ccccc7c6)n5)c34)cc2)cc1]

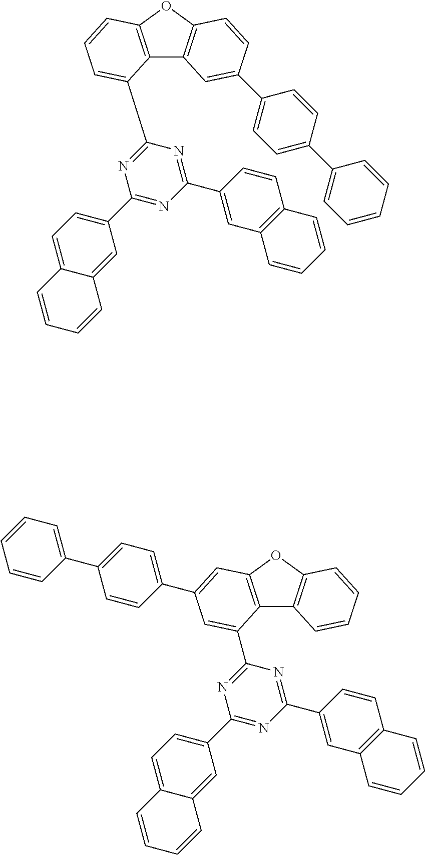 [SMILES: c1ccc(-c2ccc(-c3ccc4oc5cccc(-c6nc(-c7ccc8ccccc8c7)nc(-c7ccc8ccccc8c7)n6)c5c4c3)cc2)cc1 ; c1ccc(-c2ccc(-c3cc(-c4nc(-c5ccc6ccccc6c5)nc(-c5ccc6ccccc6c5)n4)c4c(c3)oc3ccccc34)cc2)cc1]

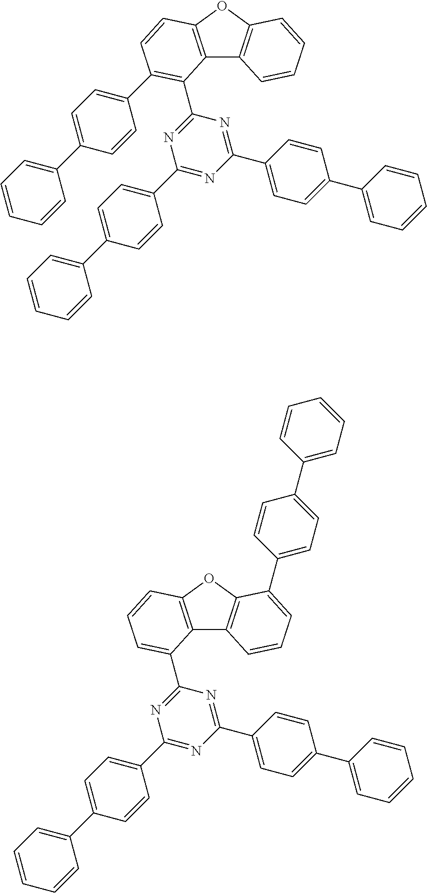 [SMILES: c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1]

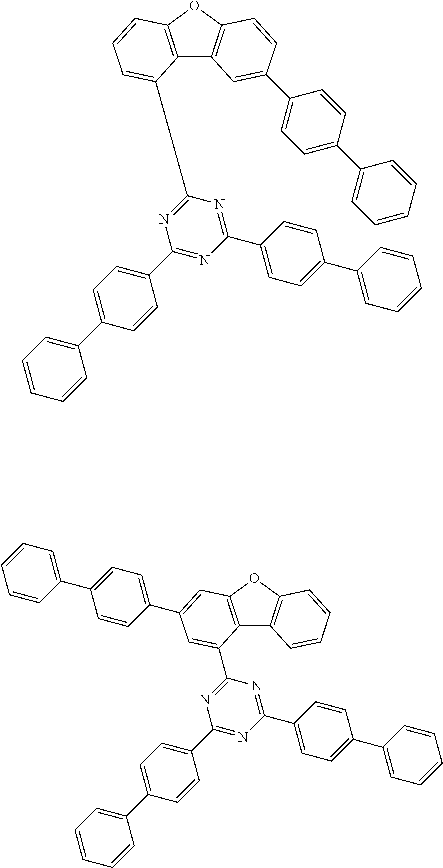 [SMILES: c1ccc(-c2ccc(-c3ccc4oc5cccc(-c6nc(-c7ccc(-c8ccccc8)cc7)nc(-c7ccc(-c8ccccc8)cc7)n6)c5c4c3)cc2)cc1 ; c1ccc(-c2ccc(-c3cc(-c4nc(-c5ccc(-c6ccccc6)cc5)nc(-c5ccc(-c6ccccc6)cc5)n4)c4c(c3)oc3ccccc34)cc2)cc1]

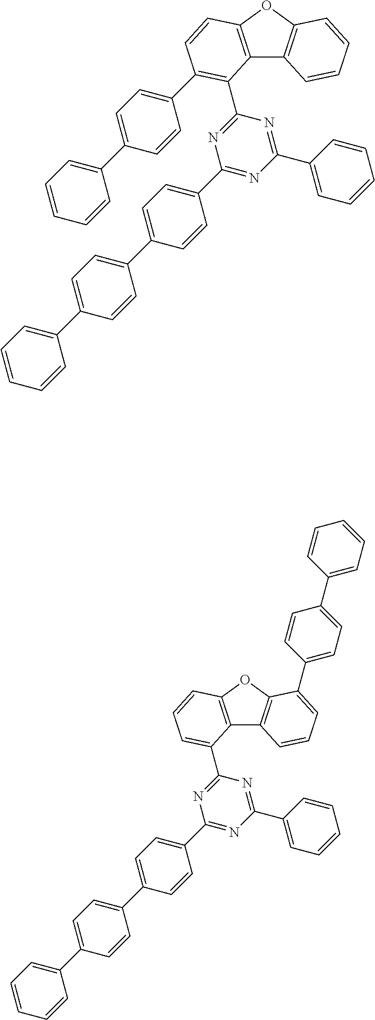 [SMILES: c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

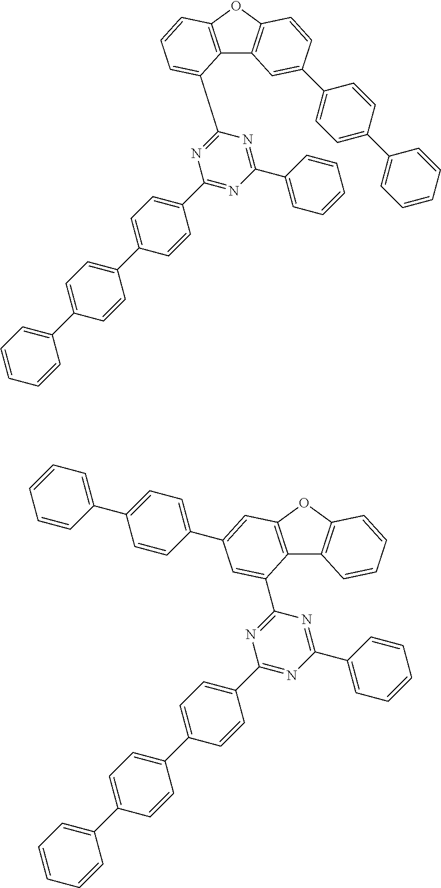 [SMILES: c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccccc5)nc(-c5cc(-c6ccc(-c7ccccc7)cc6)cc6oc7ccccc7c56)n4)cc3)cc2)cc1]

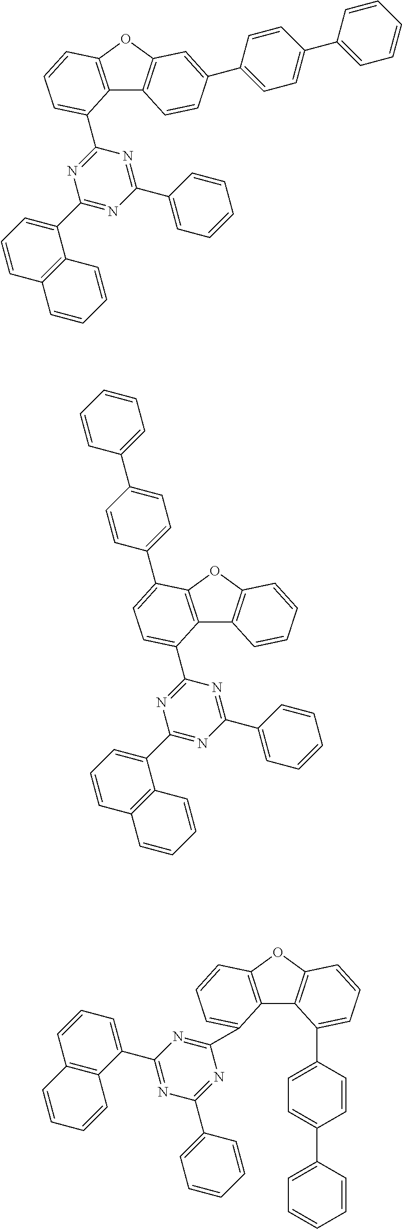 [SMILES: c1ccc(-c2ccc(-c3ccc4c(c3)oc3cccc(-c5nc(-c6ccccc6)nc(-c6cccc7ccccc67)n5)c34)cc2)cc1 ; c1ccc(-c2ccc(-c3ccc(-c4nc(-c5ccccc5)nc(-c5cccc6ccccc56)n4)c4c3oc3ccccc34)cc2)cc1 ; c1ccc(-c2ccc(-c3cccc4oc5cccc(-c6nc(-c7ccccc7)nc(-c7cccc8ccccc78)n6)c5c34)cc2)cc1]

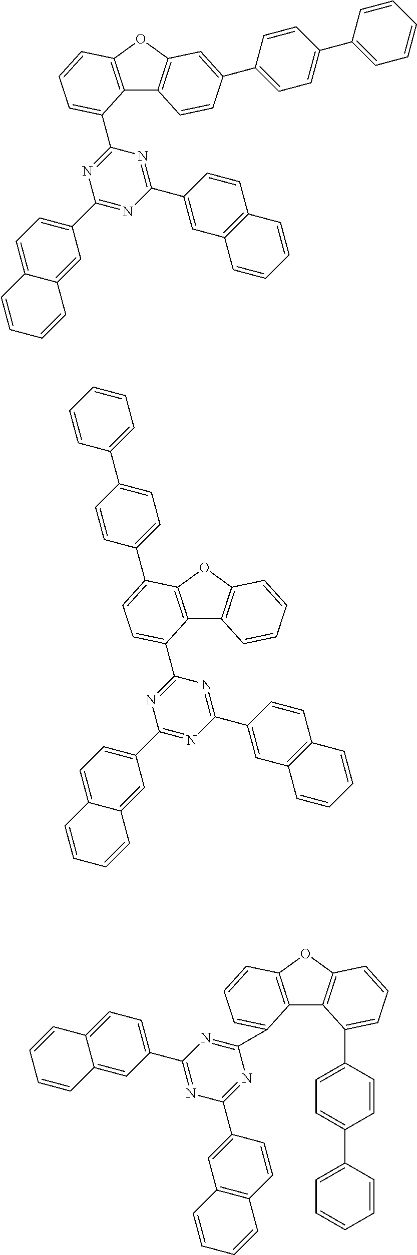 [SMILES: c1ccc(-c2ccc(-c3ccc4c(c3)oc3cccc(-c5nc(-c6ccc7ccccc7c6)nc(-c6ccc7ccccc7c6)n5)c34)cc2)cc1 ; c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1]

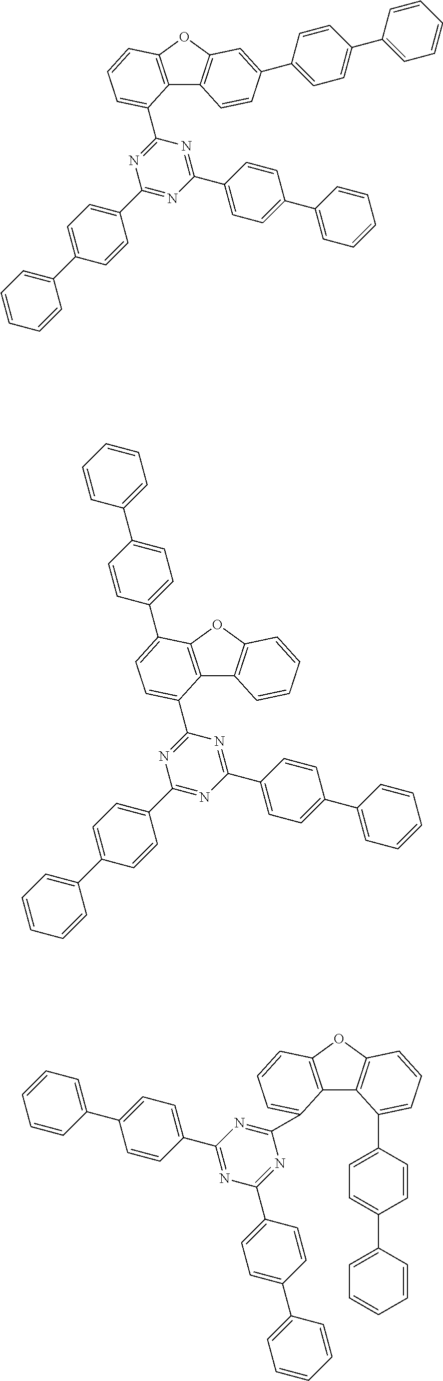 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1]

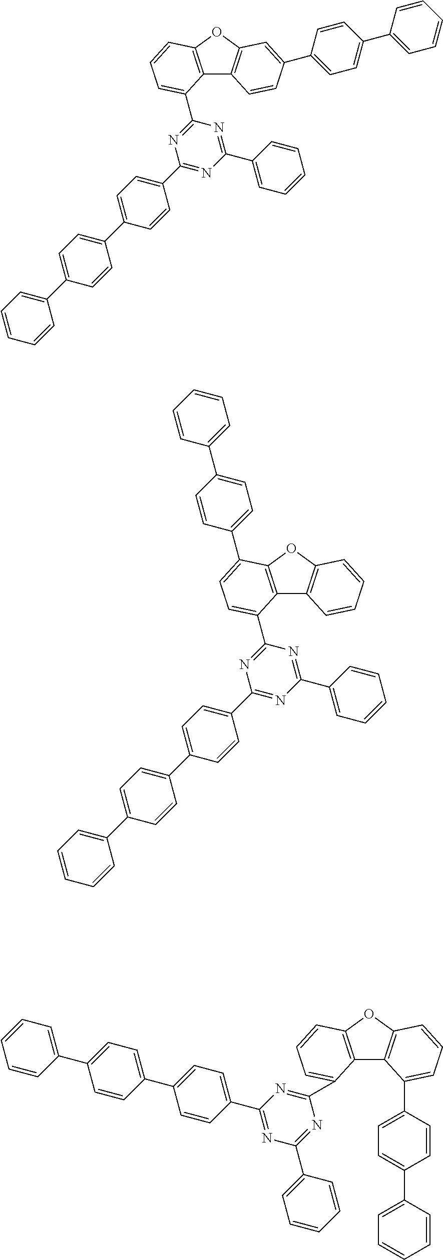 [SMILES: c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

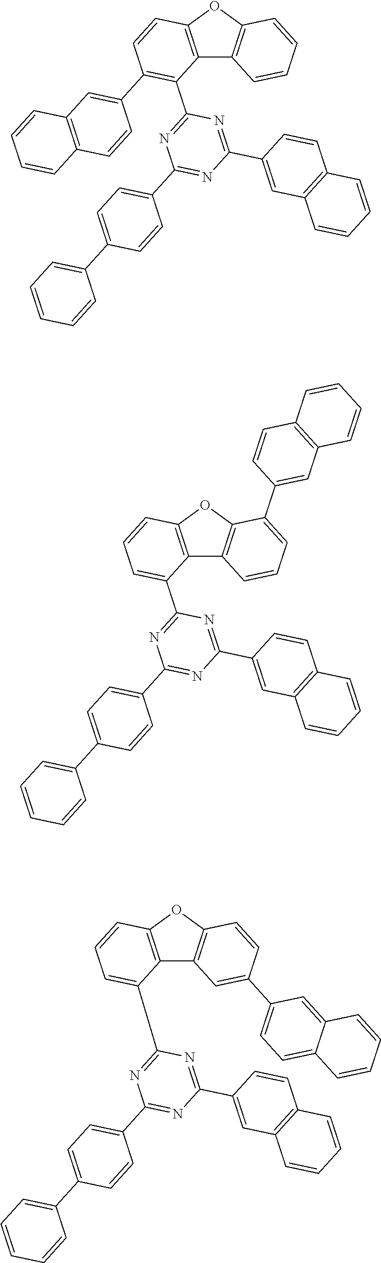 [SMILES: c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1 ; c1ccc(-c2ccc(-c3nc(-c4ccc5ccccc5c4)nc(-c4cccc5oc6ccc(-c7ccc8ccccc8c7)cc6c45)n3)cc2)cc1]

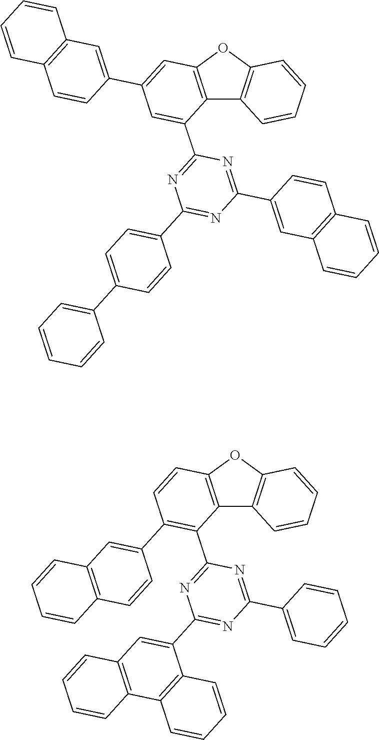 [SMILES: c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2nc(-c3cc4ccccc4c4ccccc34)nc(-c3c(-c4ccc5ccccc5c4)ccc4oc5ccccc5c34)n2)cc1]

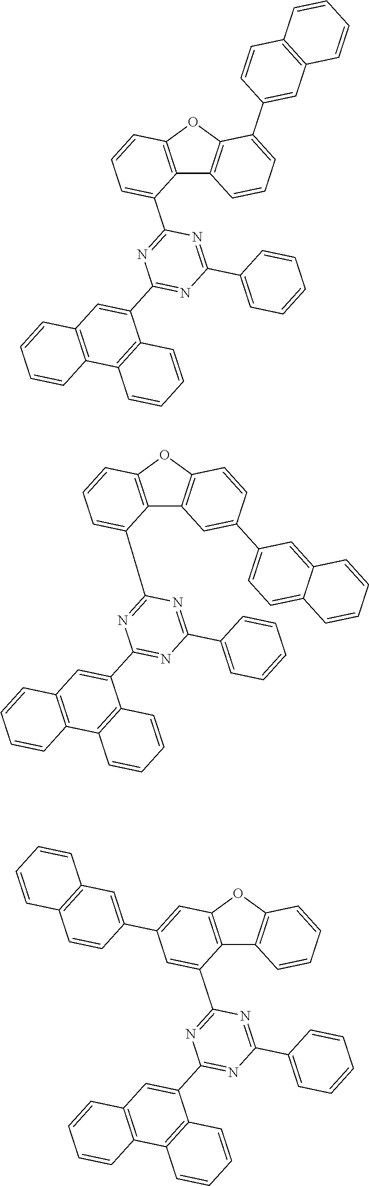 [SMILES: c1ccc(-c2nc(-c3cc4ccccc4c4ccccc34)nc(-c3cccc4oc5c(-c6ccc7ccccc7c6)cccc5c34)n2)cc1 ; c1ccc(-c2nc(-c3cc4ccccc4c4ccccc34)nc(-c3cccc4oc5ccc(-c6ccc7ccccc7c6)cc5c34)n2)cc1 ; c1ccc(-c2nc(-c3cc4ccccc4c4ccccc34)nc(-c3cc(-c4ccc5ccccc5c4)cc4oc5ccccc5c34)n2)cc1]

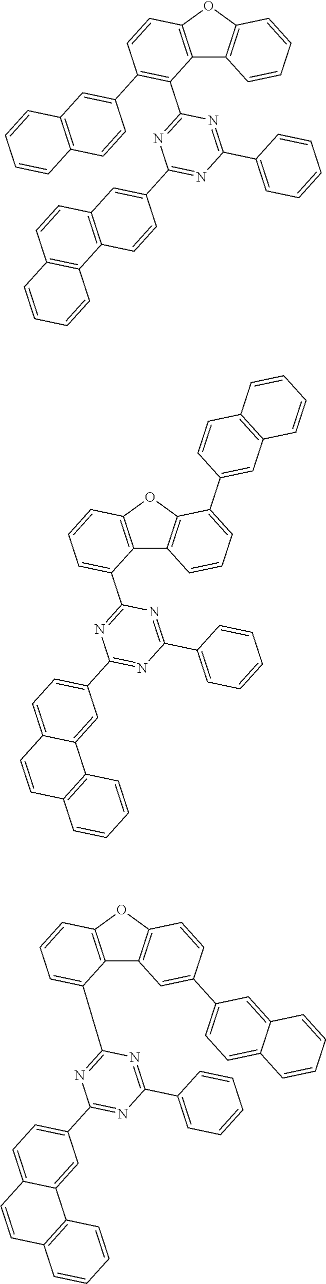 [SMILES: c1ccc(-c2nc(-c3ccc4c(ccc5ccccc54)c3)nc(-c3c(-c4ccc5ccccc5c4)ccc4oc5ccccc5c34)n2)cc1 ; c1ccc(-c2nc(-c3ccc4ccc5ccccc5c4c3)nc(-c3cccc4oc5c(-c6ccc7ccccc7c6)cccc5c34)n2)cc1 ; c1ccc(-c2nc(-c3ccc4ccc5ccccc5c4c3)nc(-c3cccc4oc5ccc(-c6ccc7ccccc7c6)cc5c34)n2)cc1]

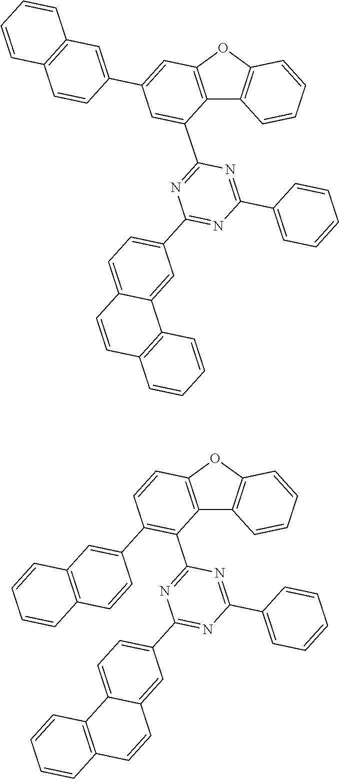 [SMILES: c1ccc(-c2nc(-c3ccc4ccc5ccccc5c4c3)nc(-c3cc(-c4ccc5ccccc5c4)cc4oc5ccccc5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

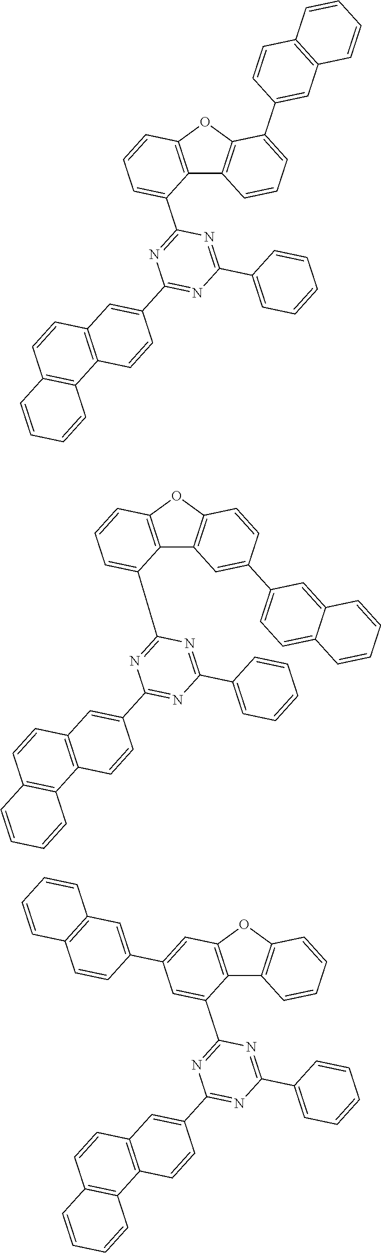

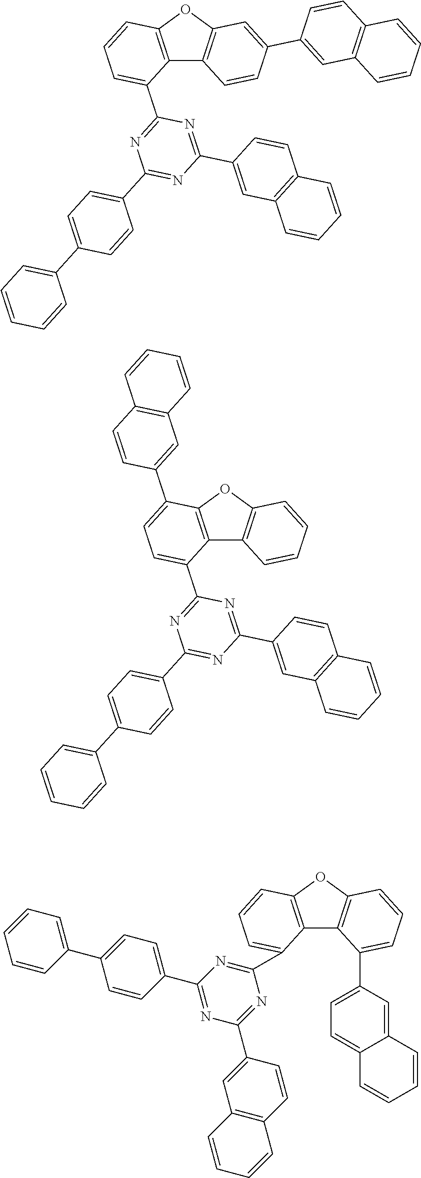 [SMILES: c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1 ; c1ccc(-c2ccc(-c3nc(-c4ccc5ccccc5c4)nc(-c4cccc5oc6cccc(-c7ccc8ccccc8c7)c6c45)n3)cc2)cc1]

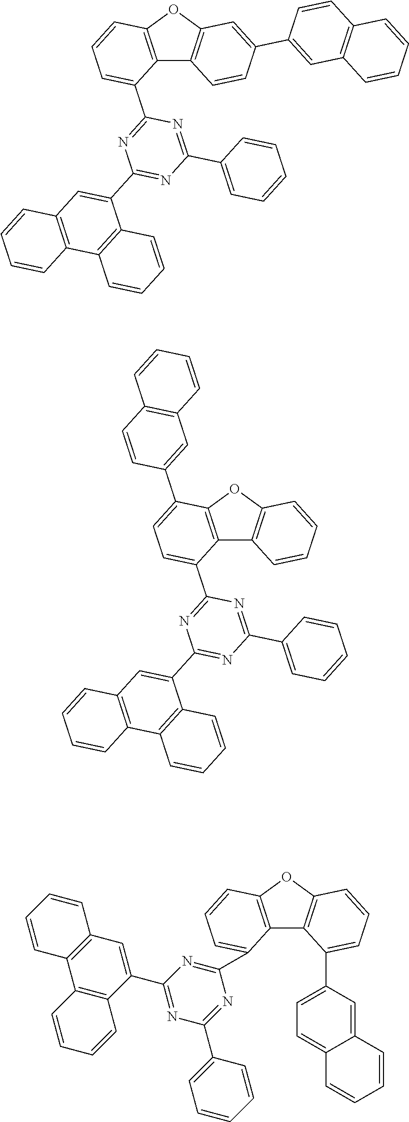 [SMILES: c1ccc(-c2nc(-c3cc4ccccc4c4ccccc34)nc(-c3cccc4oc5cc(-c6ccc7ccccc7c6)ccc5c34)n2)cc1 ; c1ccc(-c2nc(-c3cc4ccccc4c4ccccc34)nc(-c3ccc(-c4ccc5ccccc5c4)c4oc5ccccc5c34)n2)cc1 ; c1ccc(-c2nc(-c3cc4ccccc4c4ccccc34)nc(-c3cccc4oc5cccc(-c6ccc7ccccc7c6)c5c34)n2)cc1]

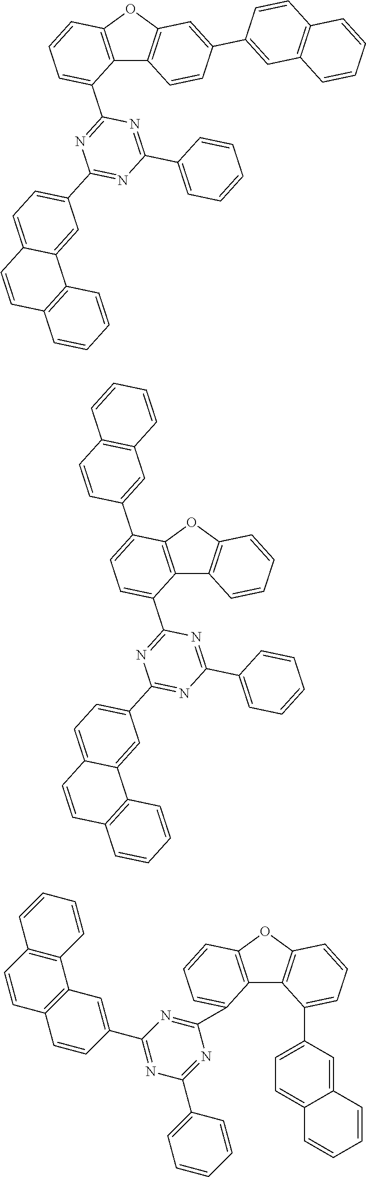

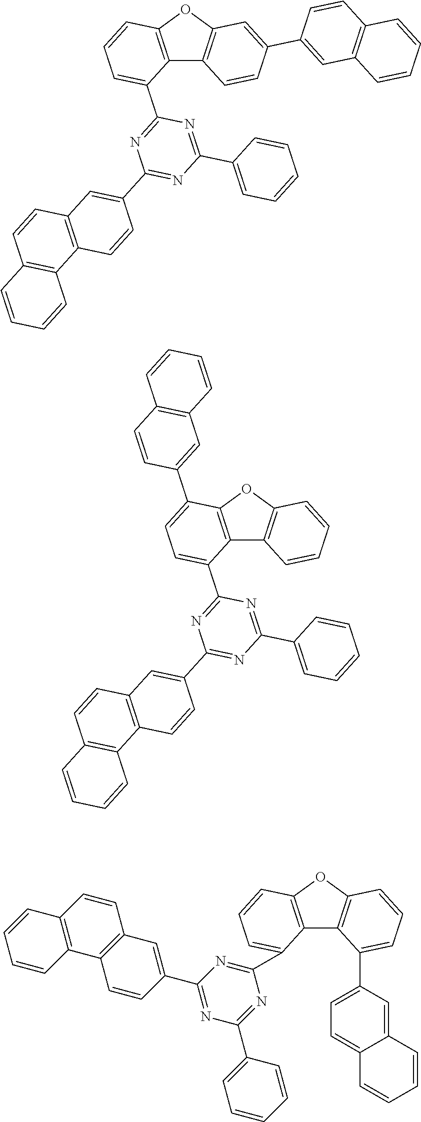 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1]

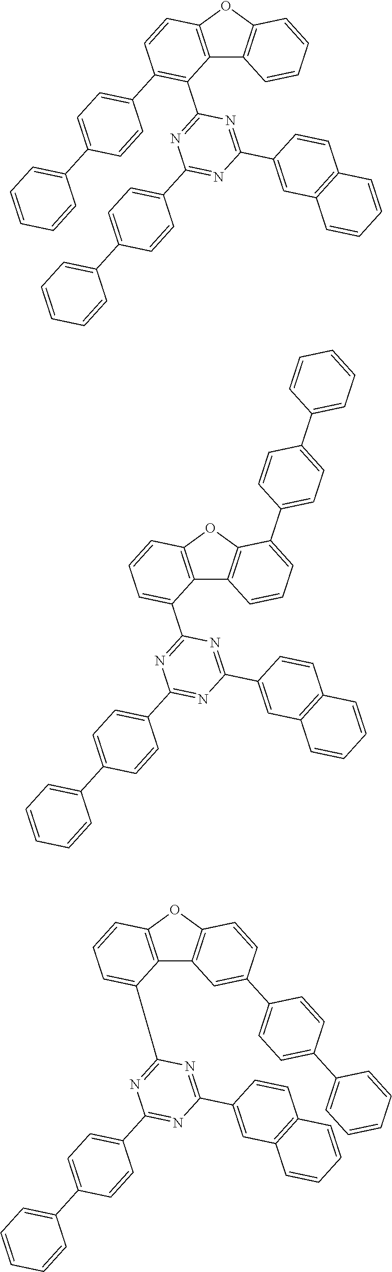 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1]

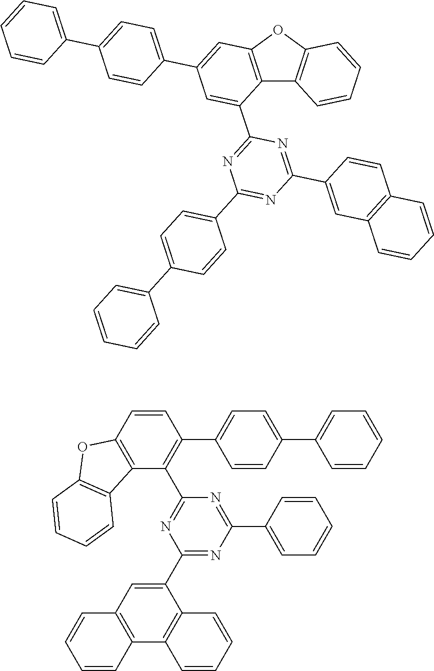 [SMILES: c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4oc5ccccc5c4c3-c3nc(-c4ccccc4)nc(-c4cc5ccccc5c5ccccc45)n3)cc2)cc1]

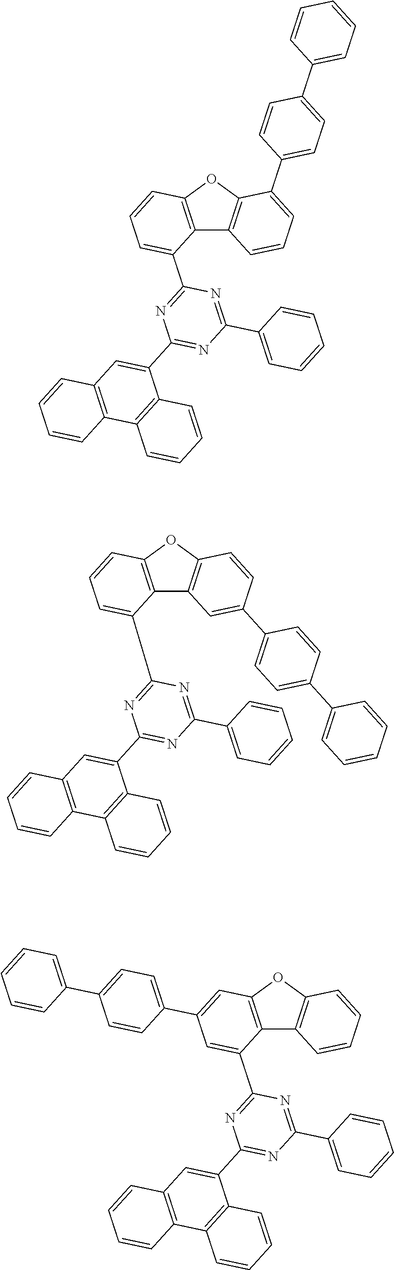 [SMILES: c1ccc(-c2ccc(-c3cccc4c3oc3cccc(-c5nc(-c6ccccc6)nc(-c6cc7ccccc7c7ccccc67)n5)c34)cc2)cc1 ; c1ccc(-c2ccc(-c3ccc4oc5cccc(-c6nc(-c7ccccc7)nc(-c7cc8ccccc8c8ccccc78)n6)c5c4c3)cc2)cc1 ; c1ccc(-c2ccc(-c3cc(-c4nc(-c5ccccc5)nc(-c5cc6ccccc6c6ccccc56)n4)c4c(c3)oc3ccccc34)cc2)cc1]

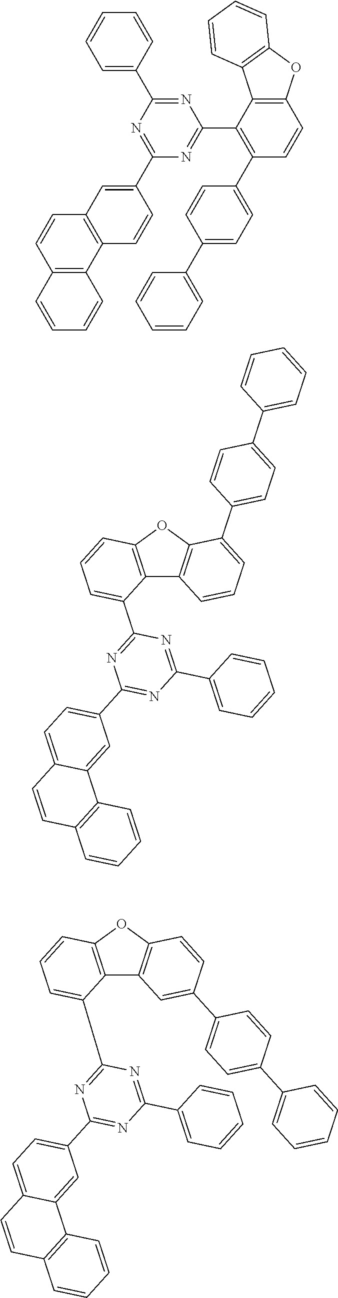 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7ccc8ccccc8c7c6)n5)c34)cc2)cc1 ; c1ccc(-c2ccc(-c3ccc4oc5cccc(-c6nc(-c7ccccc7)nc(-c7ccc8ccc9ccccc9c8c7)n6)c5c4c3)cc2)cc1]

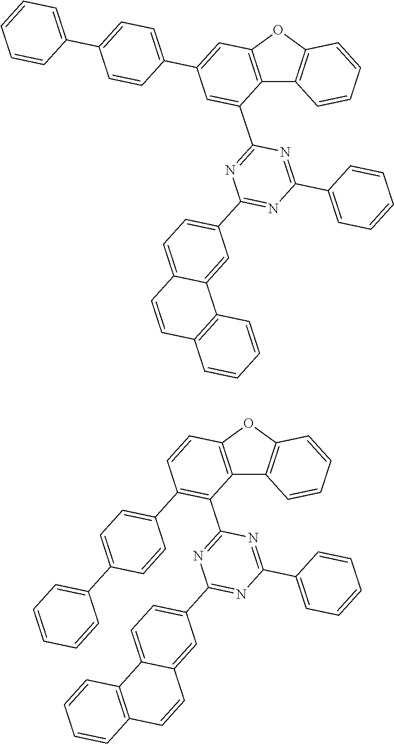 [SMILES: c1ccc(-c2ccc(-c3cc(-c4nc(-c5ccccc5)nc(-c5ccc6ccc7ccccc7c6c5)n4)c4c(c3)oc3ccccc34)cc2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

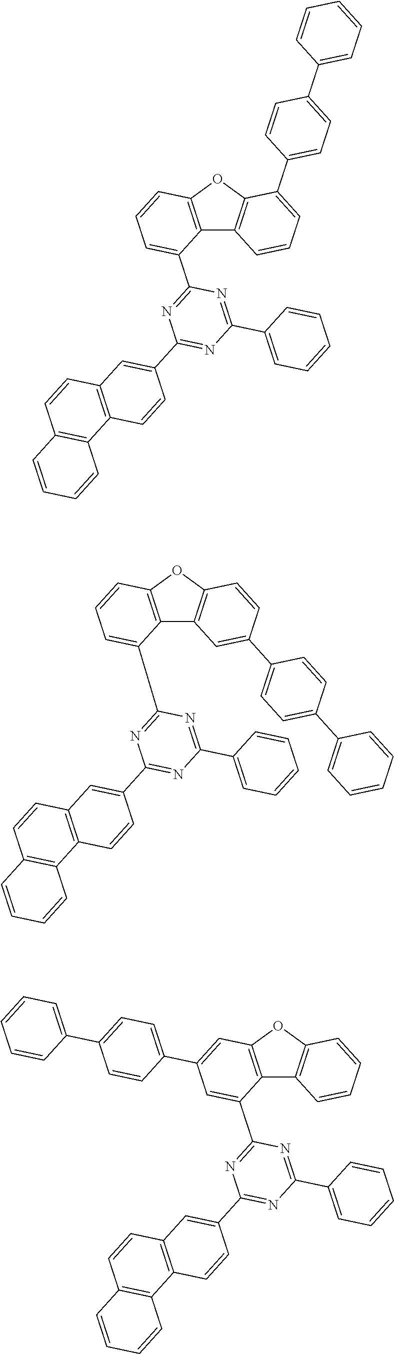 [SMILES: c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1 ; c1ccc(-c2ccc(-c3cc(-c4nc(-c5ccccc5)nc(-c5ccc6c(ccc7ccccc76)c5)n4)c4c(c3)oc3ccccc34)cc2)cc1]

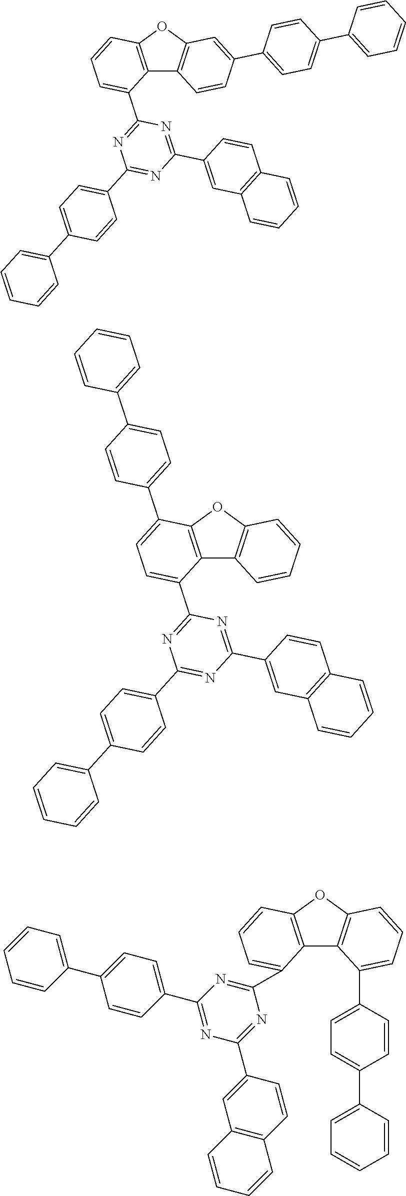 [SMILES: c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1 ; c1ccc(-c2ccc(-c3nc(-c4ccc5ccccc5c4)nc(-c4cccc5oc6cccc(-c7ccc(-c8ccccc8)cc7)c6c45)n3)cc2)cc1]

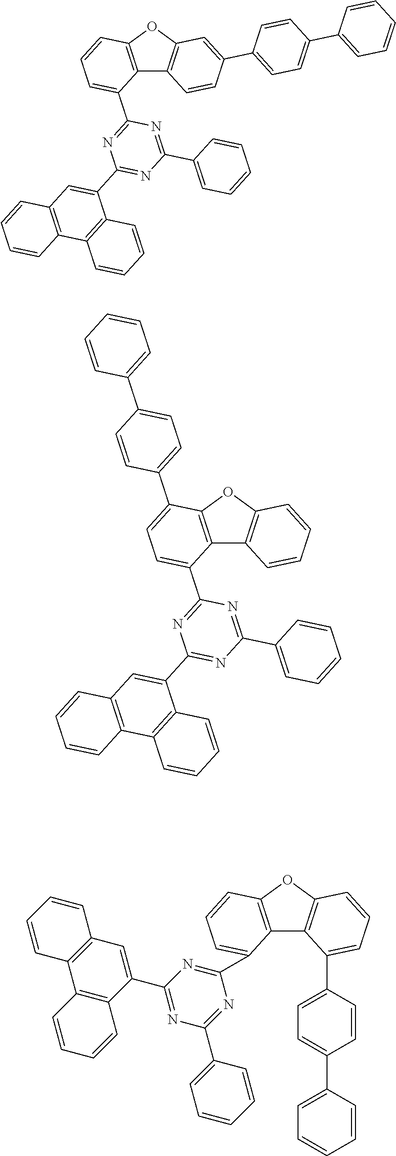

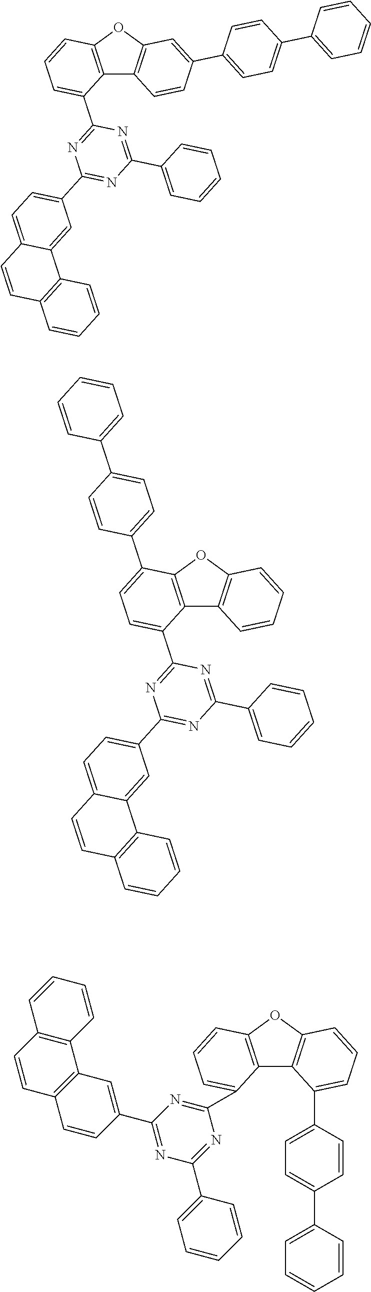

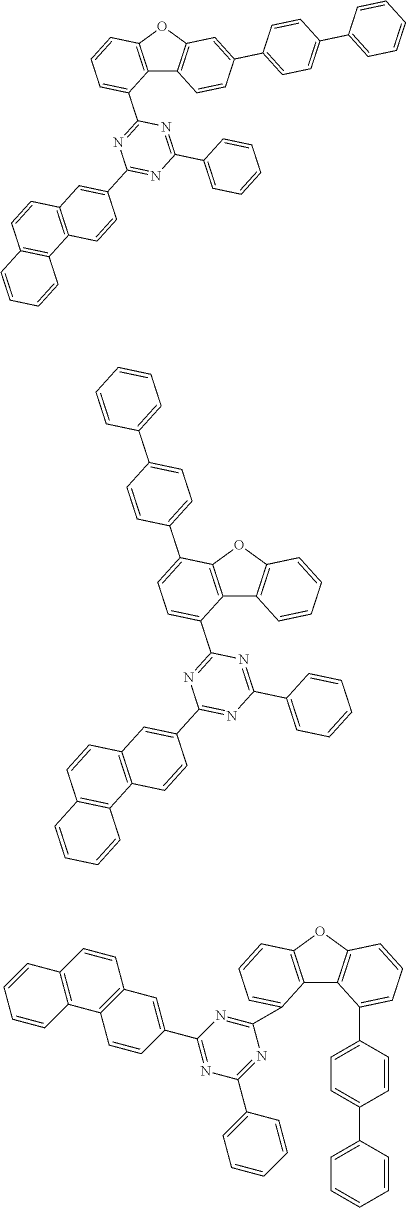 [SMILES: c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

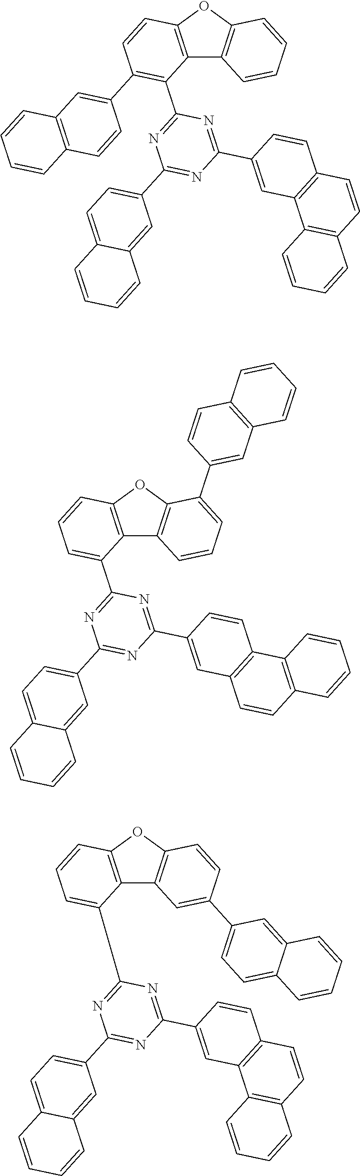 [SMILES: c1ccc2cc(-c3nc(-c4ccc5ccc6ccccc6c5c4)nc(-c4c(-c5ccc6ccccc6c5)ccc5oc6ccccc6c45)n3)ccc2c1 ; c1ccc2cc(-c3nc(-c4ccc5c(ccc6ccccc65)c4)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7ccc8ccc9ccccc9c8c7)n6)c5c4c3)ccc2c1]

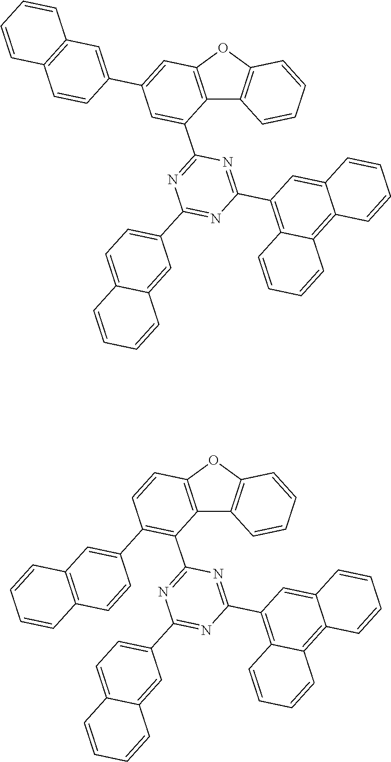 [SMILES: c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3nc(-c4cc5ccccc5c5ccccc45)nc(-c4c(-c5ccc6ccccc6c5)ccc5oc6ccccc6c45)n3)ccc2c1]

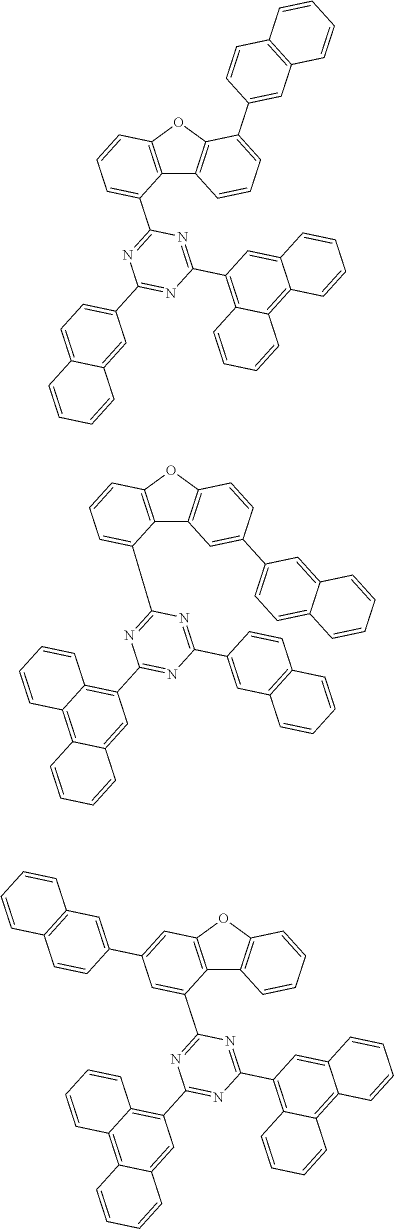 [SMILES: c1ccc2cc(-c3nc(-c4cc5ccccc5c5ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cc8ccccc8c8ccccc78)n6)c5c4c3)ccc2c1 ; c1ccc2cc(-c3cc(-c4nc(-c5cc6ccccc6c6ccccc56)nc(-c5cc6ccccc6c6ccccc56)n4)c4c(c3)oc3ccccc34)ccc2c1]

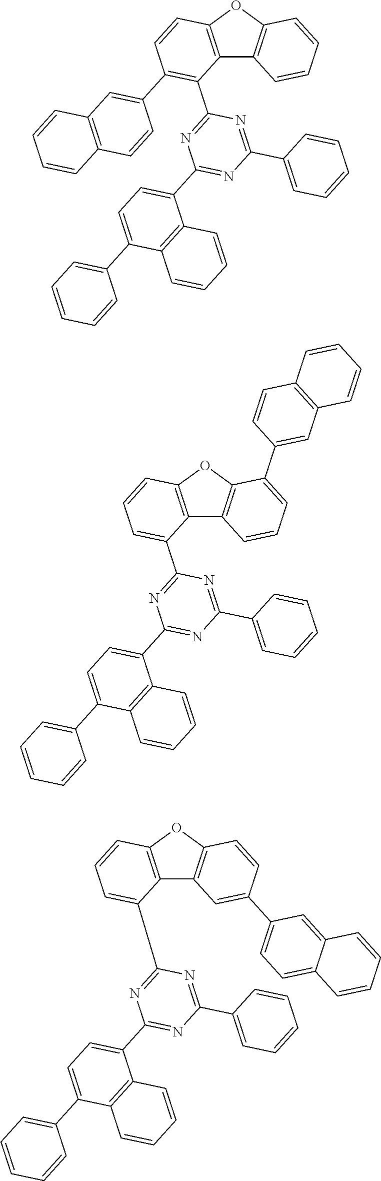 [SMILES: c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1 ; c1ccc(-c2nc(-c3ccc(-c4ccccc4)c4ccccc34)nc(-c3cccc4oc5ccc(-c6ccc7ccccc7c6)cc5c34)n2)cc1]

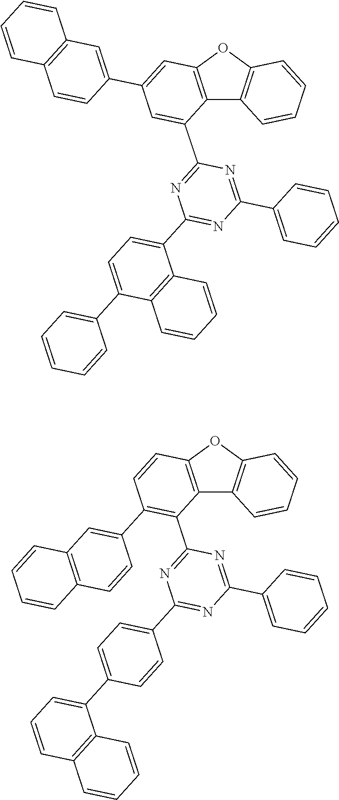 [SMILES: c1ccc(-c2nc(-c3ccc(-c4ccccc4)c4ccccc34)nc(-c3cc(-c4ccc5ccccc5c4)cc4oc5ccccc5c34)n2)cc1 ; c1ccc(-c2nc(-c3ccc(-c4cccc5ccccc45)cc3)nc(-c3c(-c4ccc5ccccc5c4)ccc4oc5ccccc5c34)n2)cc1]

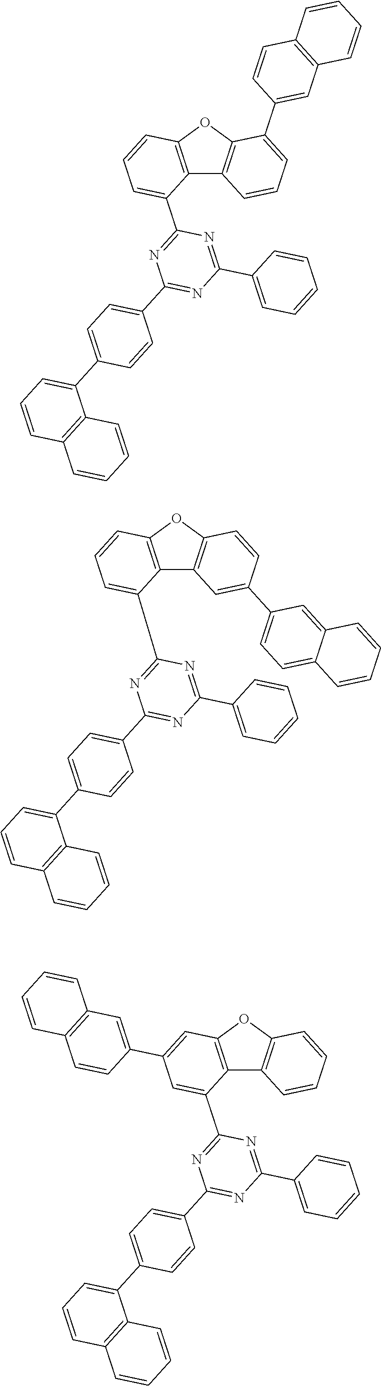 [SMILES: c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

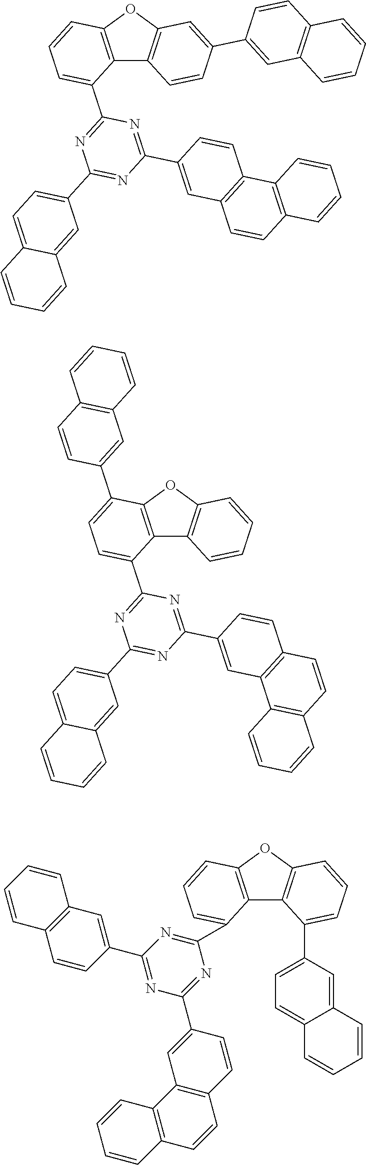 [SMILES: c1ccc2cc(-c3ccc4c(c3)oc3cccc(-c5nc(-c6ccc7ccccc7c6)nc(-c6ccc7c(ccc8ccccc87)c6)n5)c34)ccc2c1 ; c1ccc2cc(-c3nc(-c4ccc5ccc6ccccc6c5c4)nc(-c4ccc(-c5ccc6ccccc6c5)c5oc6ccccc6c45)n3)ccc2c1 ; c1ccc2cc(-c3nc(-c4ccc5ccc6ccccc6c5c4)nc(-c4cccc5oc6cccc(-c7ccc8ccccc8c7)c6c45)n3)ccc2c1]

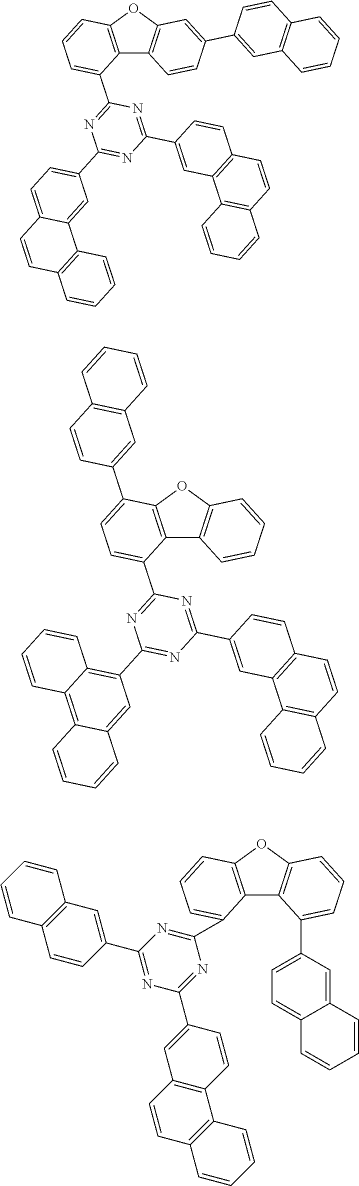

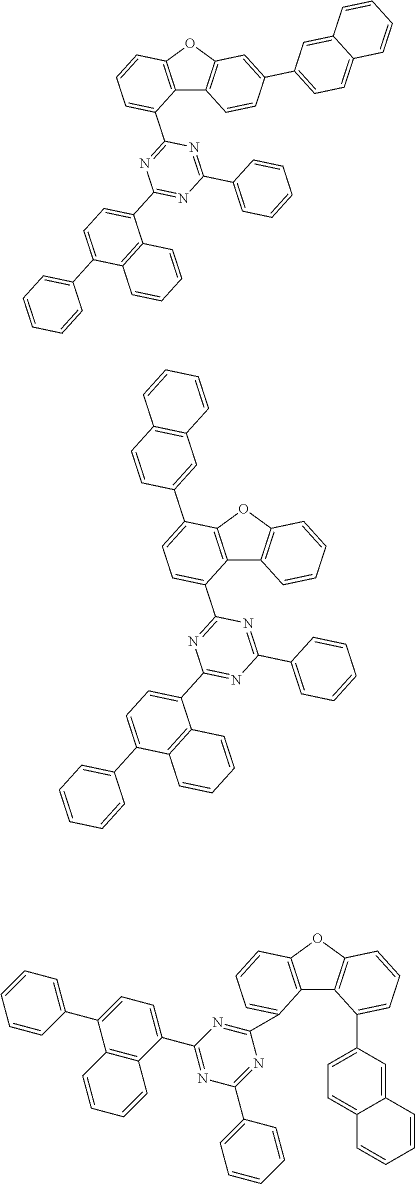 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1]

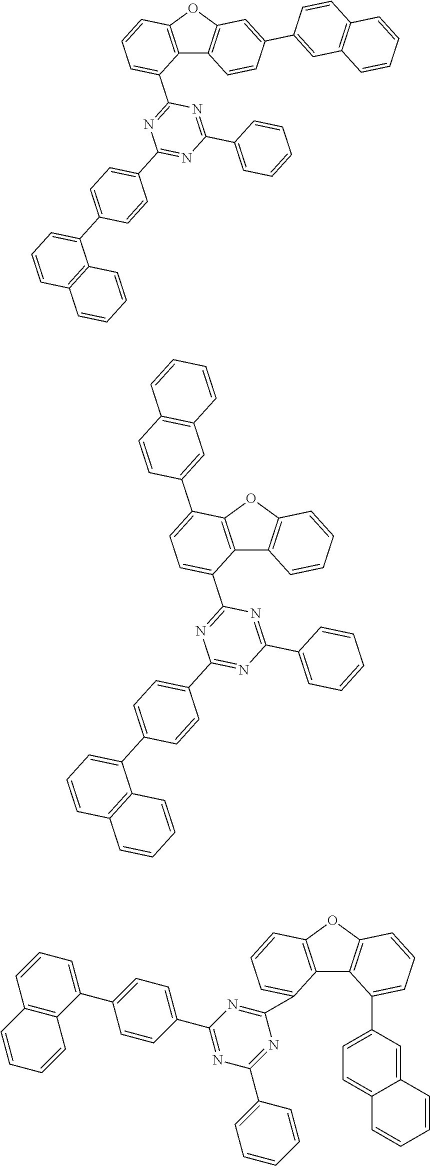 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1]

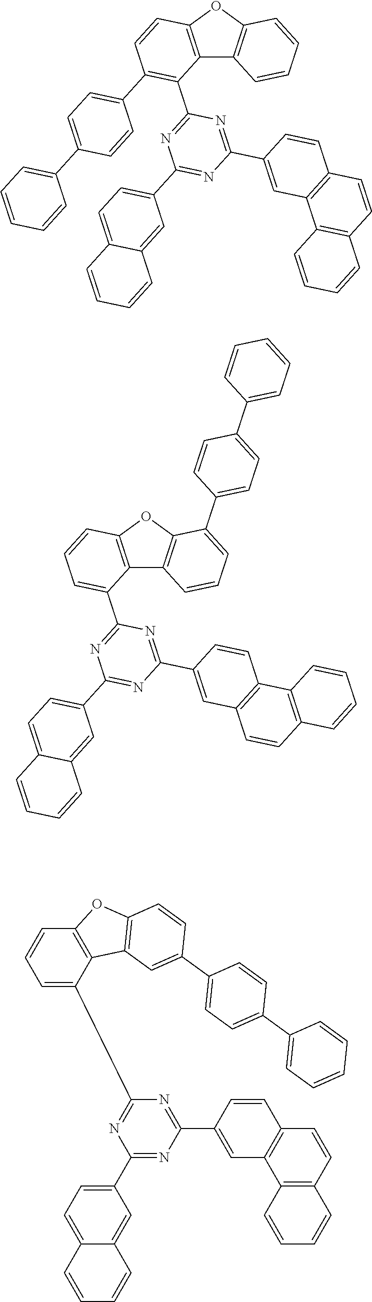 [SMILES: c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1 ; c1ccc(-c2ccc(-c3ccc4oc5cccc(-c6nc(-c7ccc8ccccc8c7)nc(-c7ccc8ccc9ccccc9c8c7)n6)c5c4c3)cc2)cc1]

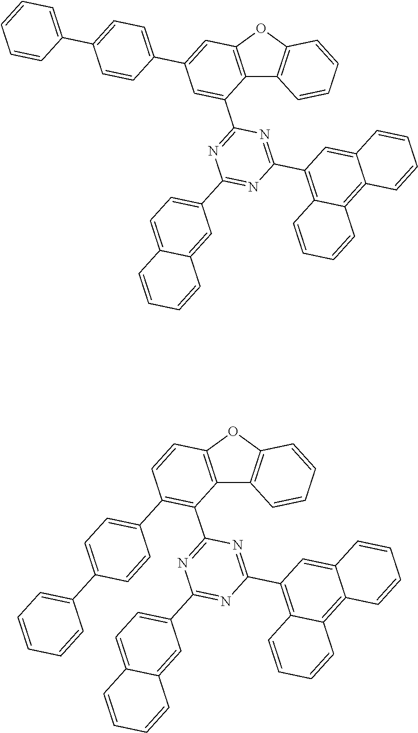 [SMILES: c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

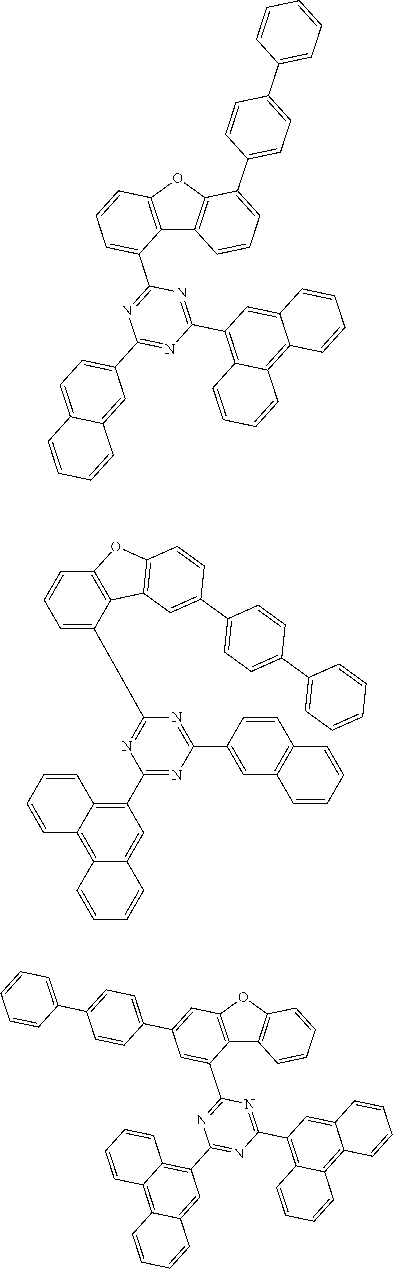 [SMILES: c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1 ; c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1]

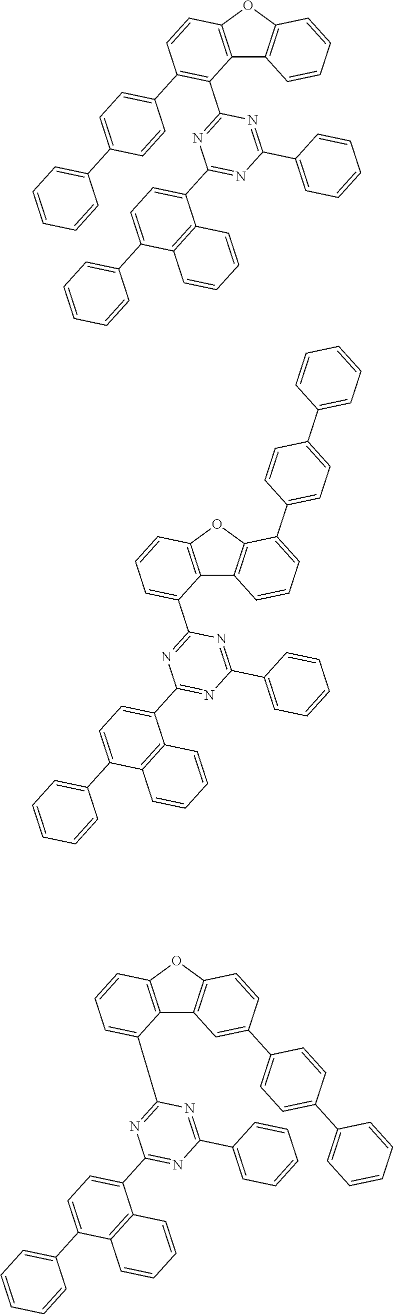 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1]

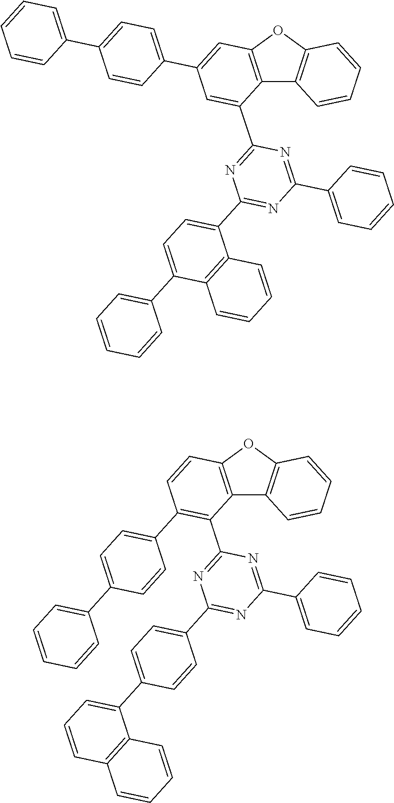 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1]

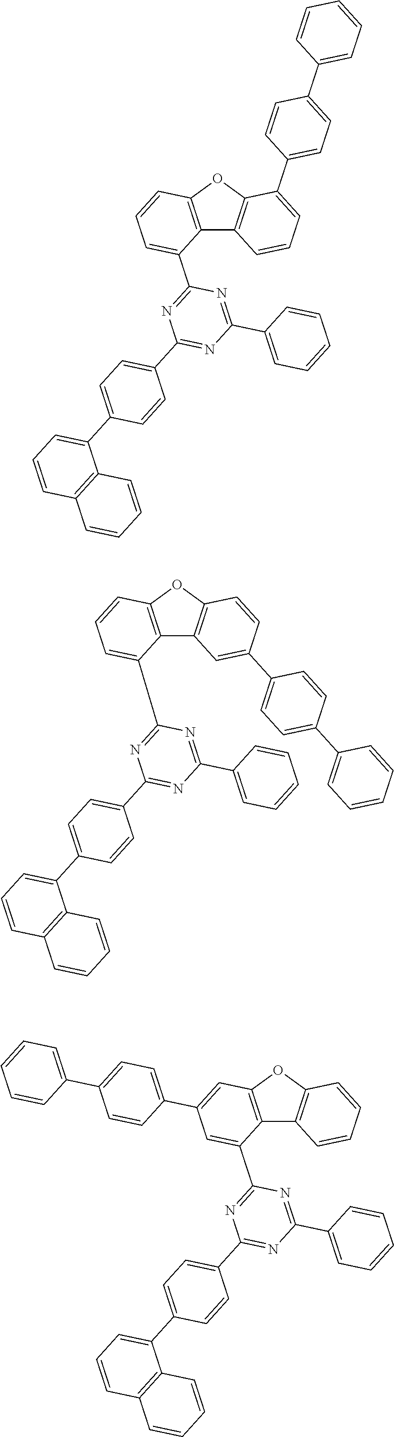 [SMILES: c1ccc(-c2ccc(-c3cccc4c3oc3cccc(-c5nc(-c6ccccc6)nc(-c6ccc(-c7cccc8ccccc78)cc6)n5)c34)cc2)cc1 ; c1ccc(-c2ccc(-c3ccc4oc5cccc(-c6nc(-c7ccccc7)nc(-c7ccc(-c8cccc9ccccc89)cc7)n6)c5c4c3)cc2)cc1 ; c1ccc(-c2ccc(-c3cc(-c4nc(-c5ccccc5)nc(-c5ccc(-c6cccc7ccccc67)cc5)n4)c4c(c3)oc3ccccc34)cc2)cc1]

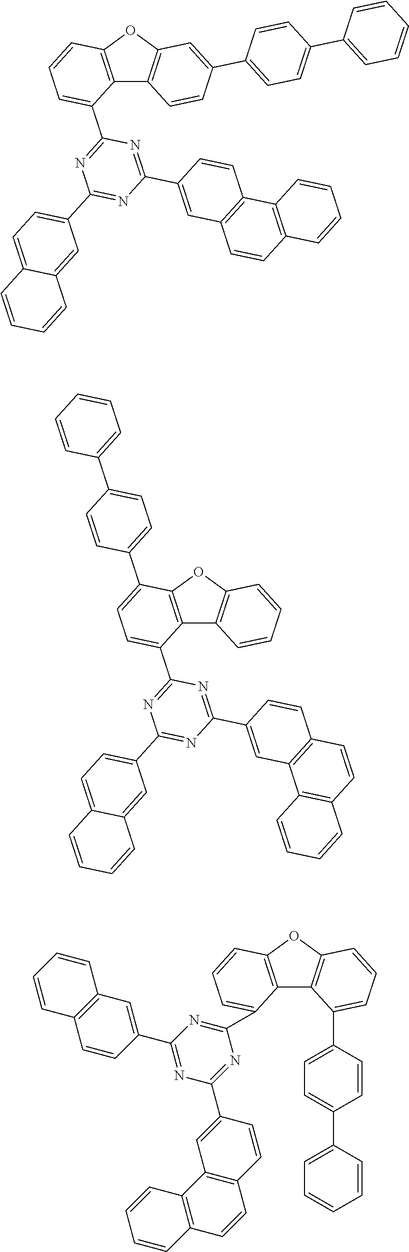 [SMILES: c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1 ; c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1]

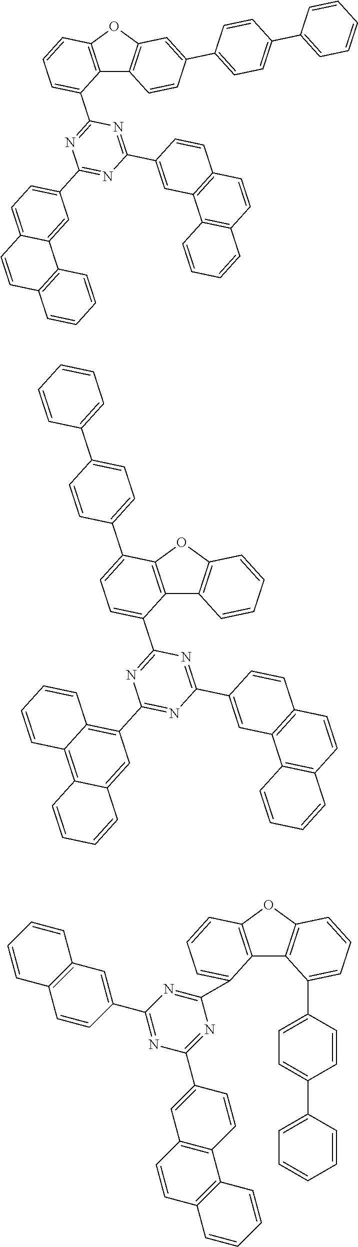

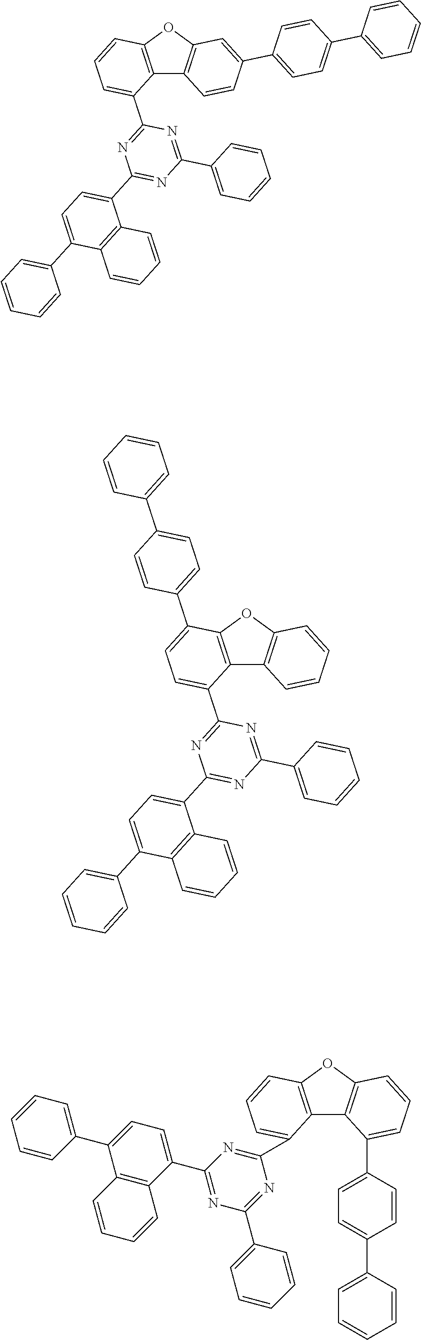 [SMILES: c1ccc(-c2ccc(-c3ccc4c(c3)oc3cccc(-c5nc(-c6ccccc6)nc(-c6ccc(-c7ccccc7)c7ccccc67)n5)c34)cc2)cc1 ; c1ccc(-c2ccc(-c3ccc(-c4nc(-c5ccccc5)nc(-c5ccc(-c6ccccc6)c6ccccc56)n4)c4c3oc3ccccc34)cc2)cc1 ; c1ccc(-c2ccc(-c3cccc4oc5cccc(-c6nc(-c7ccccc7)nc(-c7ccc(-c8ccccc8)c8ccccc78)n6)c5c34)cc2)cc1]

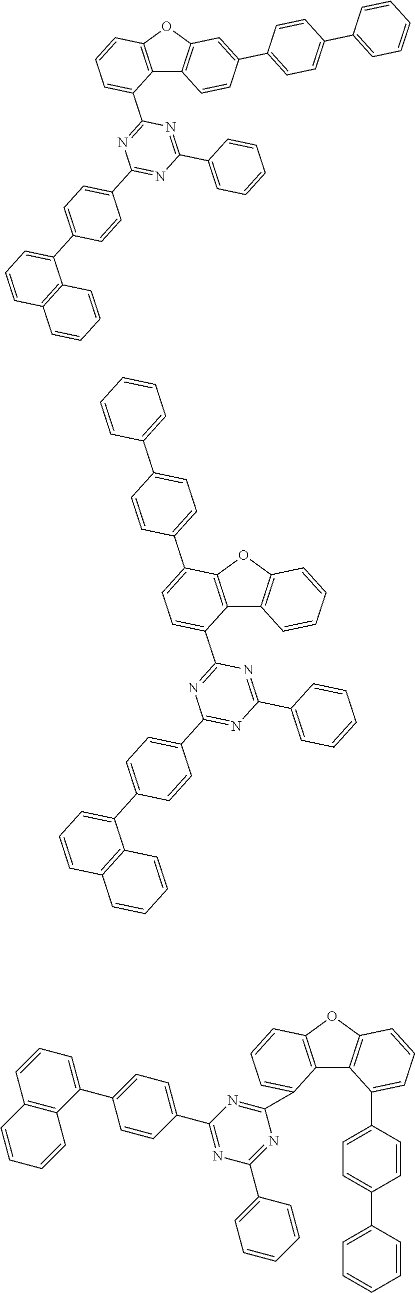 [SMILES: c1ccc(-c2ccc(-c3ccc4c(c3)oc3cccc(-c5nc(-c6ccccc6)nc(-c6ccc(-c7cccc8ccccc78)cc6)n5)c34)cc2)cc1 ; c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1]

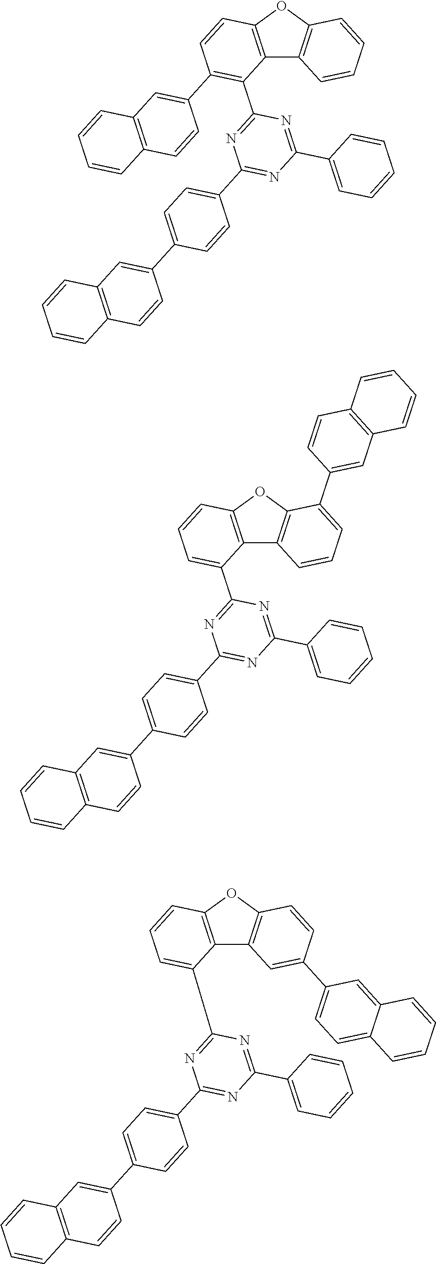 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1]

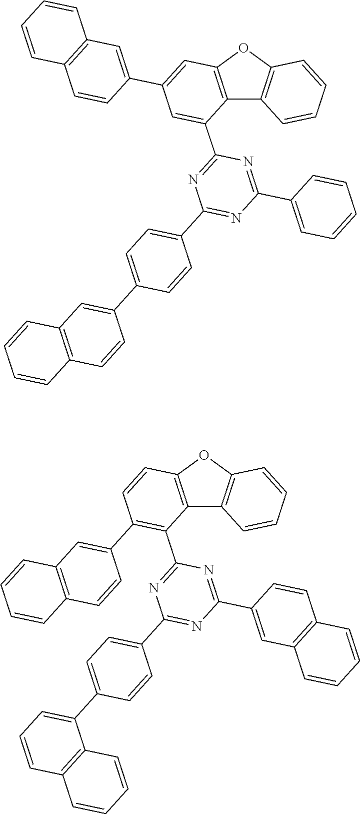 [SMILES: c1ccc(-c2nc(-c3ccc(-c4ccc5ccccc5c4)cc3)nc(-c3cc(-c4ccc5ccccc5c4)cc4oc5ccccc5c34)n2)cc1 ; c1ccc2cc(-c3nc(-c4ccc(-c5cccc6ccccc56)cc4)nc(-c4c(-c5ccc6ccccc6c5)ccc5oc6ccccc6c45)n3)ccc2c1]

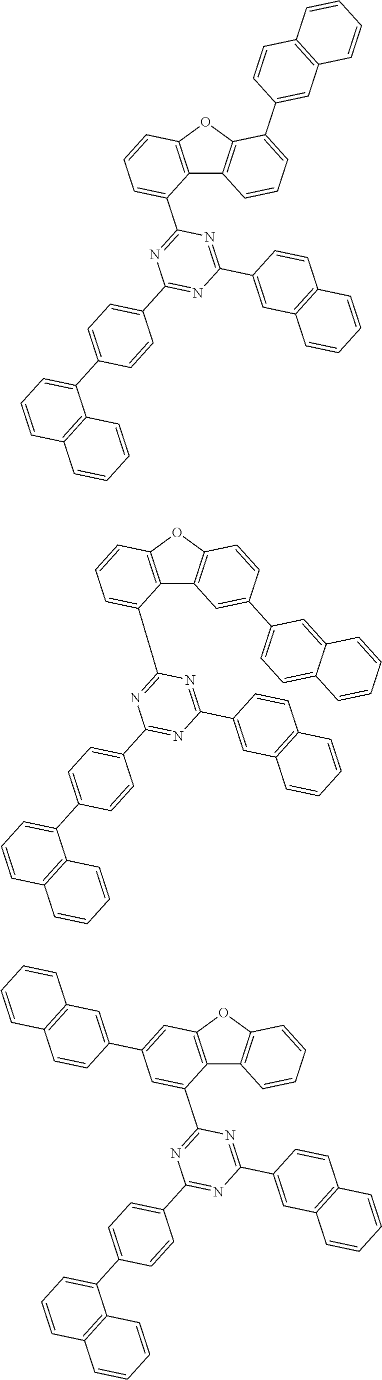 [SMILES: c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1]

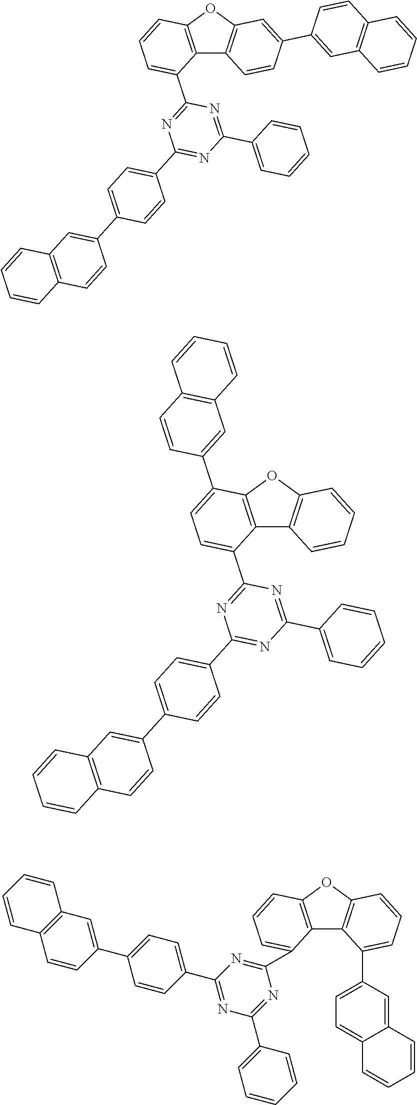 [SMILES: c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1 ; c1ccc(-c2nc(-c3ccc(-c4ccc5ccccc5c4)cc3)nc(-c3cccc4oc5cccc(-c6ccc7ccccc7c6)c5c34)n2)cc1]

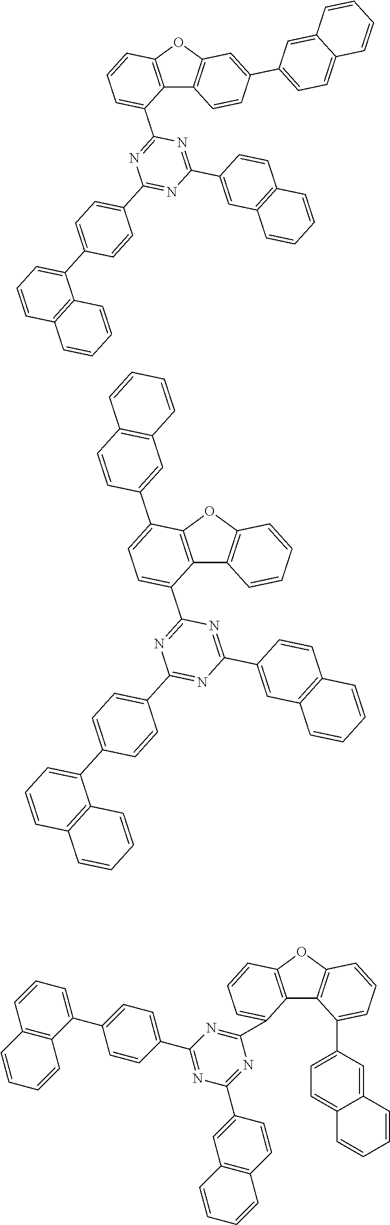 [SMILES: c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1]

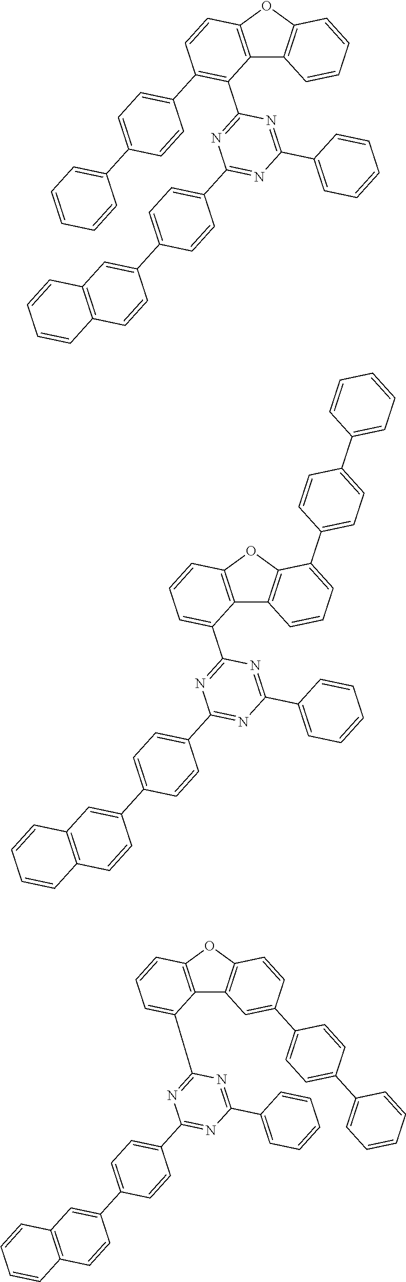 [SMILES: c1ccc(-c2ccc(-c3ccc4oc5ccccc5c4c3-c3nc(-c4ccccc4)nc(-c4ccc(-c5ccc6ccccc6c5)cc4)n3)cc2)cc1 ; c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1]

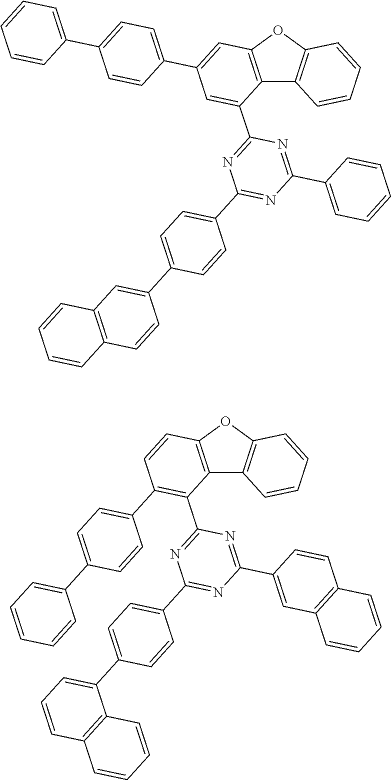 [SMILES: c1ccc(-c2ccc(-c3cc(-c4nc(-c5ccccc5)nc(-c5ccc(-c6ccc7ccccc7c6)cc5)n4)c4c(c3)oc3ccccc34)cc2)cc1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

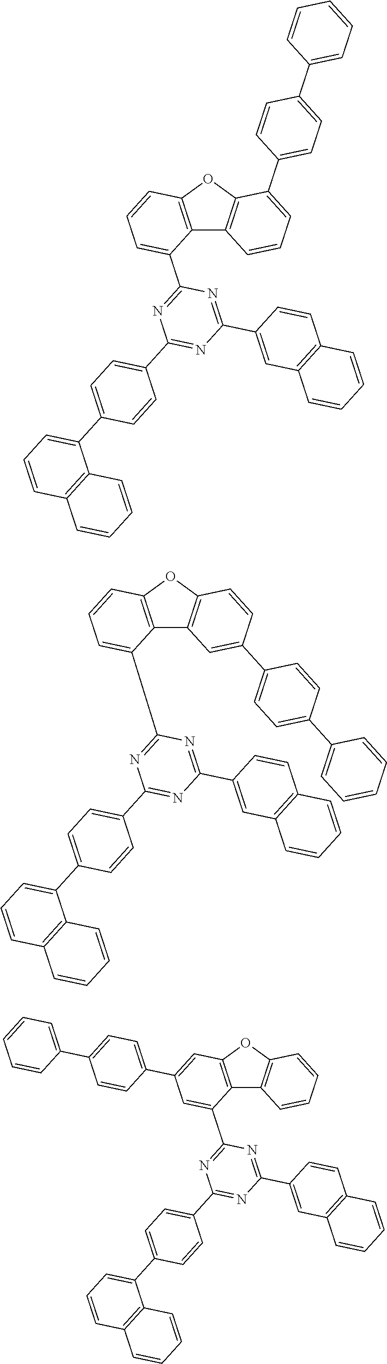

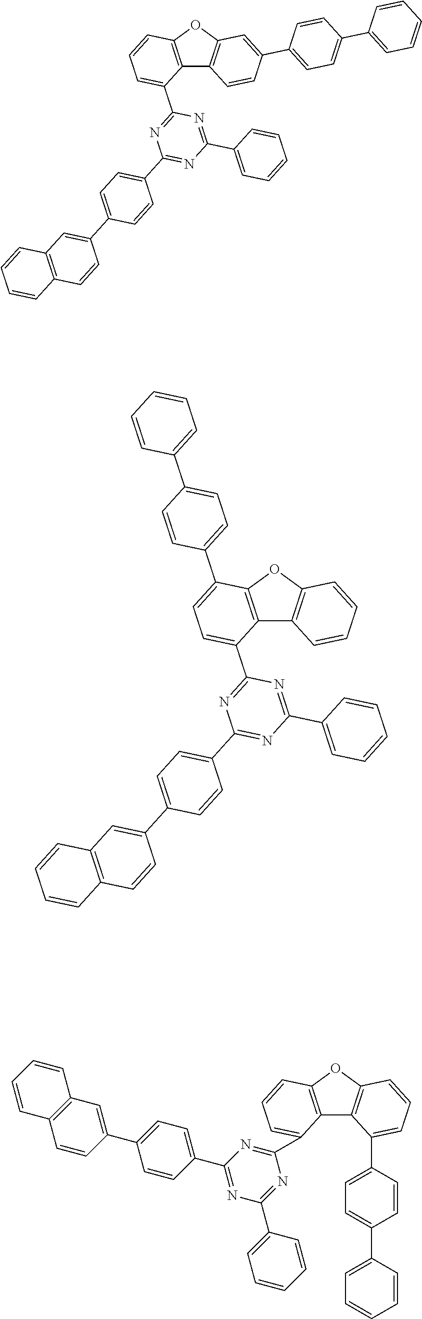 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5cccc(-c6nc(-c7ccccc7)nc(-c7ccc(-c8ccc9ccccc9c8)cc7)n6)c5c34)cc2)cc1]

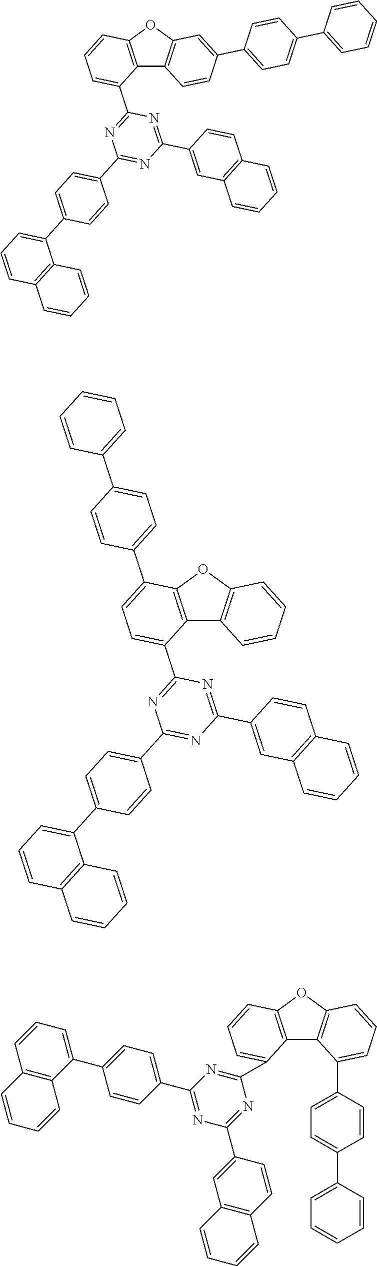 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

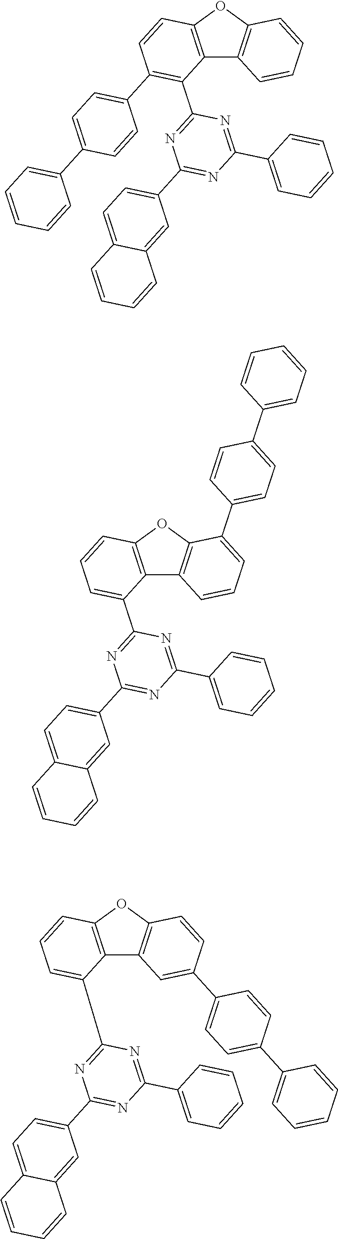 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1]

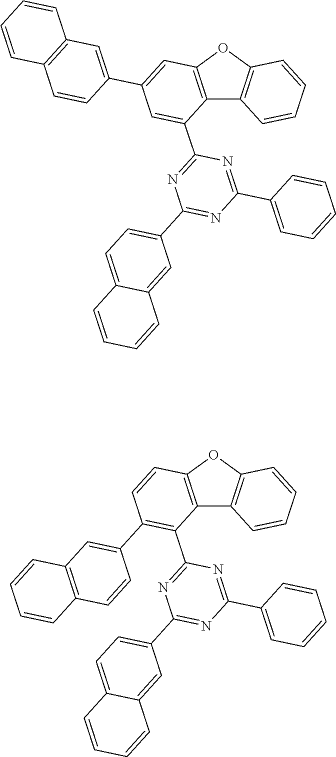 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3c(-c4ccc5ccccc5c4)ccc4oc5ccccc5c34)n2)cc1]

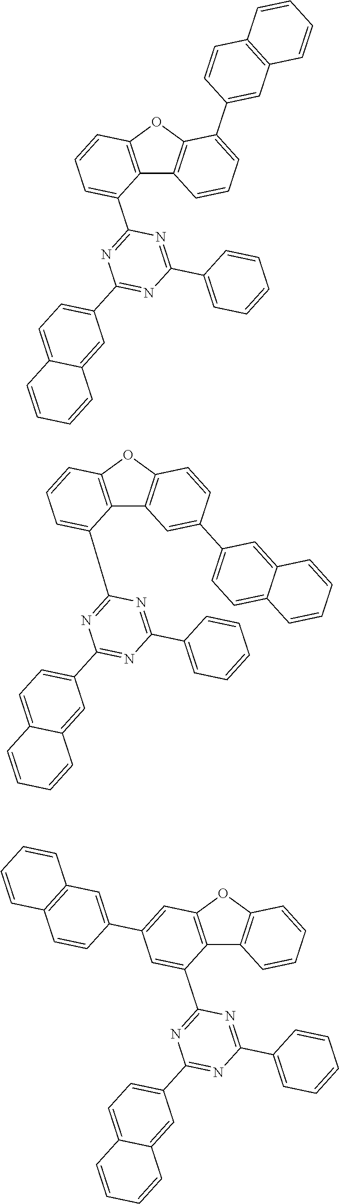

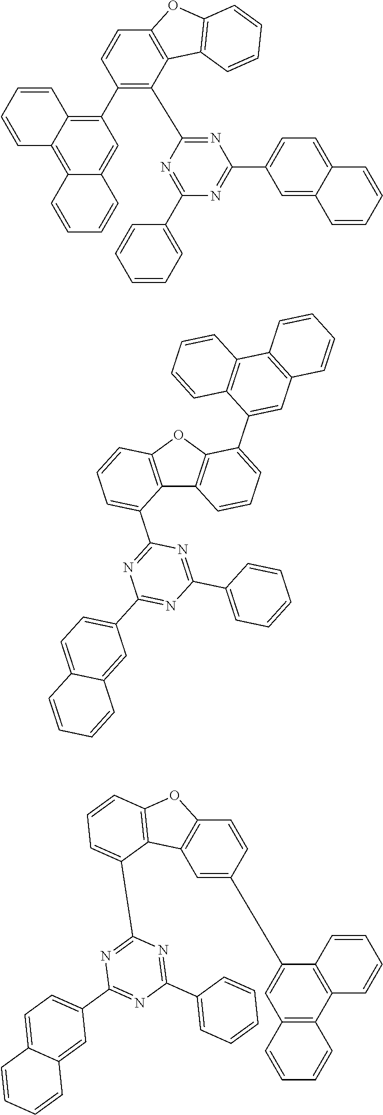 [SMILES: c1ccc(-c2nc(-c3ccc4ccccc4c3)nc(-c3c(-c4cc5ccccc5c5ccccc45)ccc4oc5ccccc5c34)n2)cc1 ; c1ccc(-c2nc(-c3ccc4ccccc4c3)nc(-c3cccc4oc5c(-c6cc7ccccc7c7ccccc67)cccc5c34)n2)cc1 ; c1ccc(-c2nc(-c3ccc4ccccc4c3)nc(-c3cccc4oc5ccc(-c6cc7ccccc7c7ccccc67)cc5c34)n2)cc1]

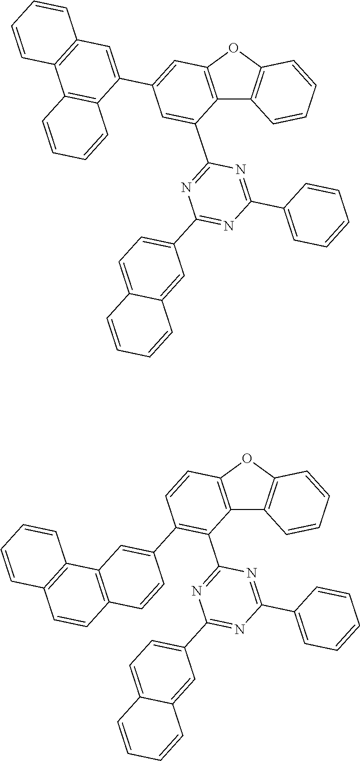 [SMILES: c1ccc(-c2nc(-c3ccc4ccccc4c3)nc(-c3cc(-c4cc5ccccc5c5ccccc45)cc4oc5ccccc5c34)n2)cc1 ; c1ccc(-c2nc(-c3ccc4ccccc4c3)nc(-c3c(-c4ccc5ccc6ccccc6c5c4)ccc4oc5ccccc5c34)n2)cc1]

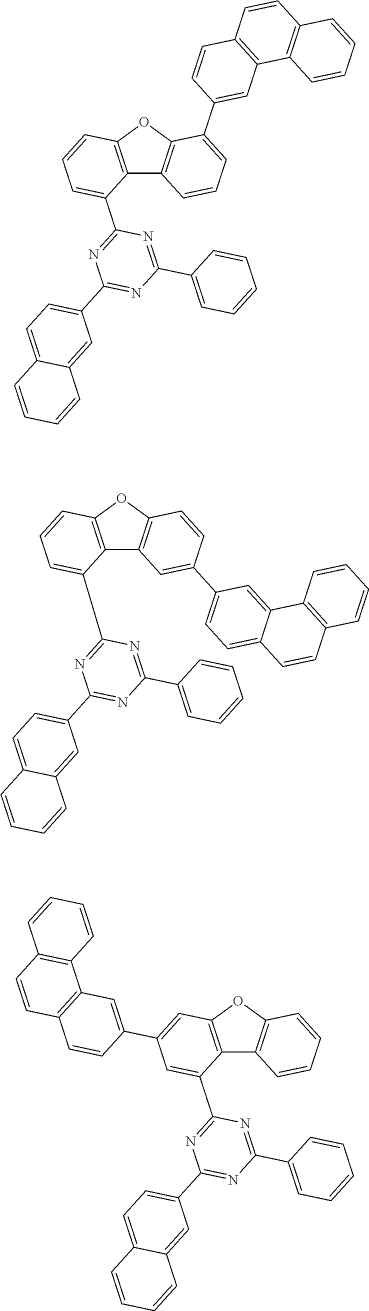 [SMILES: c1ccc(-c2nc(-c3ccc4ccccc4c3)nc(-c3cccc4oc5c(-c6ccc7ccc8ccccc8c7c6)cccc5c34)n2)cc1 ; c1ccc(-c2nc(-c3ccc4ccccc4c3)nc(-c3cccc4oc5ccc(-c6ccc7ccc8ccccc8c7c6)cc5c34)n2)cc1 ; c1ccc(-c2nc(-c3ccc4ccccc4c3)nc(-c3cc(-c4ccc5ccc6ccccc6c5c4)cc4oc5ccccc5c34)n2)cc1]

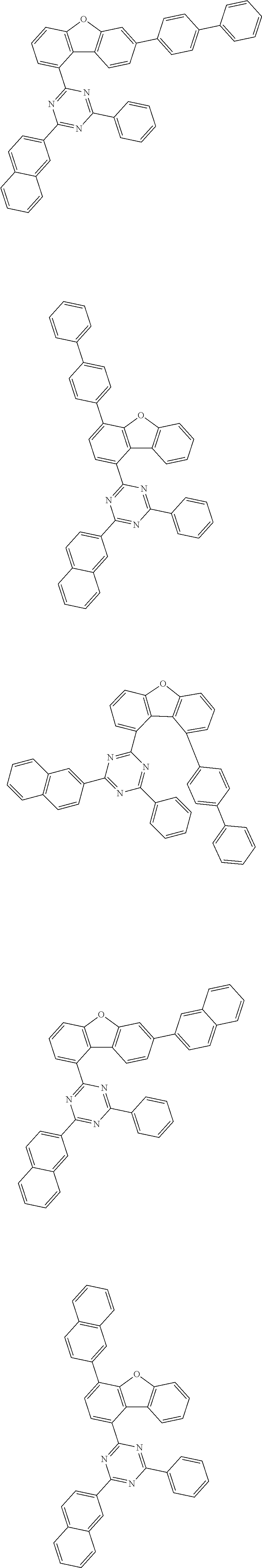 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3cccc4oc5cccc(-c6nc(-c7ccccc7)nc(-c7ccc8ccccc8c7)n6)c5c34)cc2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3ccc(-c4ccc5ccccc5c4)c4oc5ccccc5c34)n2)cc1]

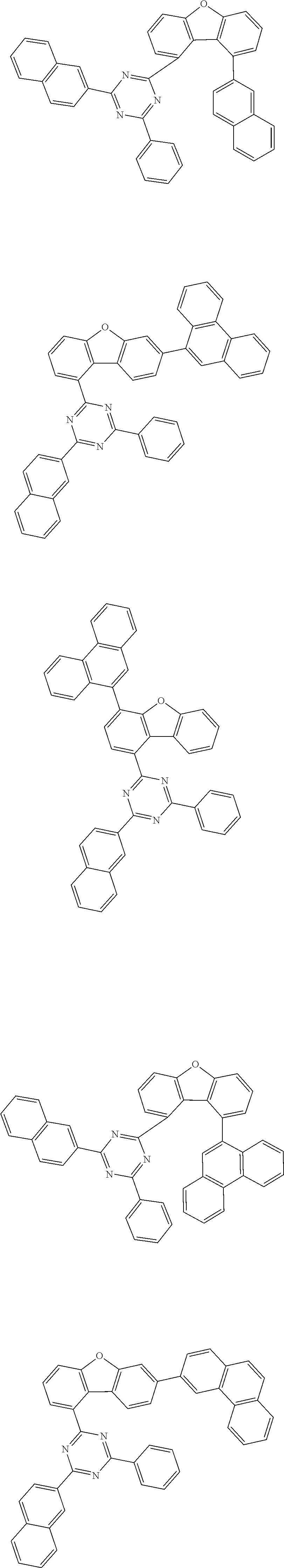 [SMILES: c1ccc(-c2nc(-c3ccc4ccccc4c3)nc(-c3cccc4oc5cccc(-c6ccc7ccccc7c6)c5c34)n2)cc1 ; c1ccc(-c2nc(-c3ccc4ccccc4c3)nc(-c3cccc4oc5cc(-c6cc7ccccc7c7ccccc67)ccc5c34)n2)cc1 ; c1ccc(-c2nc(-c3ccc4ccccc4c3)nc(-c3ccc(-c4cc5ccccc5c5ccccc45)c4oc5ccccc5c34)n2)cc1 ; c1ccc(-c2nc(-c3ccc4ccccc4c3)nc(-c3cccc4oc5cccc(-c6cc7ccccc7c7ccccc67)c5c34)n2)cc1 ; c1ccc(-c2nc(-c3ccc4ccccc4c3)nc(-c3cccc4oc5cc(-c6ccc7ccc8ccccc8c7c6)ccc5c34)n2)cc1]

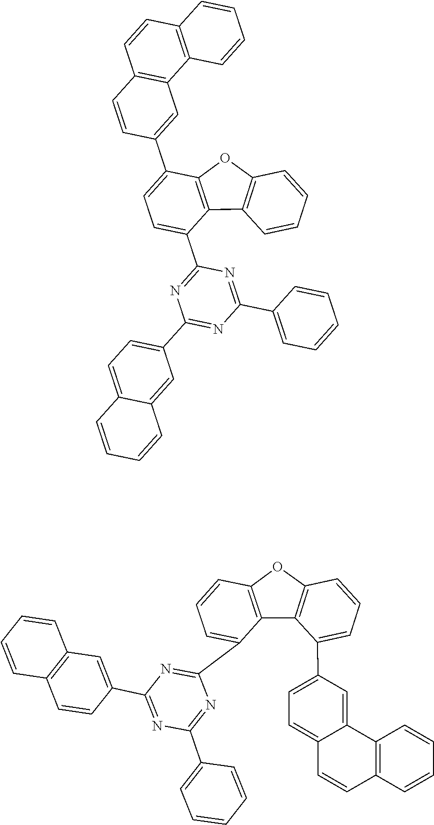 [SMILES: c1ccc(-c2nc(-c3ccc4ccccc4c3)nc(-c3ccc(-c4ccc5ccc6ccccc6c5c4)c4oc5ccccc5c34)n2)cc1 ; c1ccc(-c2nc(-c3ccc4ccccc4c3)nc(-c3cccc4oc5cccc(-c6ccc7ccc8ccccc8c7c6)c5c34)n2)cc1]

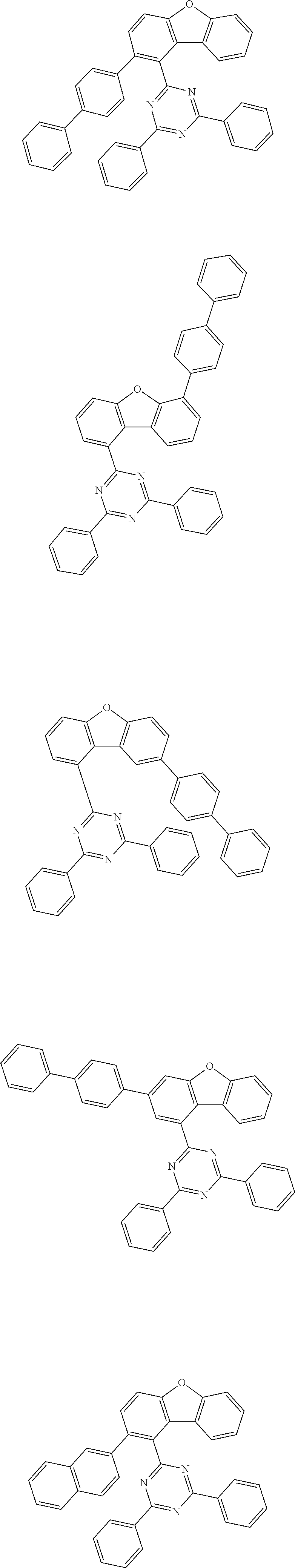 [SMILES: c1ccc(-c2ccc(-c3ccc4oc5ccccc5c4c3-c3nc(-c4ccccc4)nc(-c4ccccc4)n3)cc2)cc1 ; c1ccc(-c2ccc(-c3cccc4c3oc3cccc(-c5nc(-c6ccccc6)nc(-c6ccccc6)n5)c34)cc2)cc1 ; c1ccc(-c2ccc(-c3ccc4oc5cccc(-c6nc(-c7ccccc7)nc(-c7ccccc7)n6)c5c4c3)cc2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2nc(-c3ccccc3)nc(-c3c(-c4ccc5ccccc5c4)ccc4oc5ccccc5c34)n2)cc1]

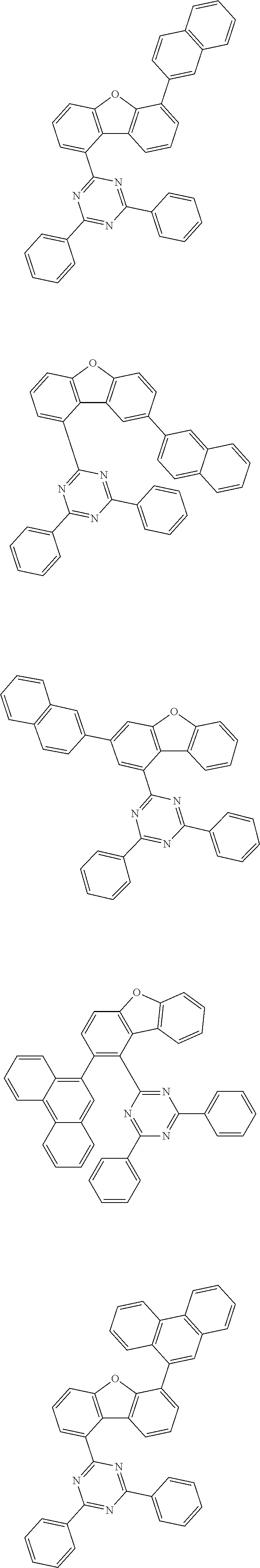 [SMILES: c1ccc(-c2nc(-c3ccccc3)nc(-c3cccc4oc5c(-c6ccc7ccccc7c6)cccc5c34)n2)cc1 ; c1ccc(-c2nc(-c3ccccc3)nc(-c3cccc4oc5ccc(-c6ccc7ccccc7c6)cc5c34)n2)cc1 ; c1ccc(-c2nc(-c3ccccc3)nc(-c3cc(-c4ccc5ccccc5c4)cc4oc5ccccc5c34)n2)cc1 ; c1ccc(-c2nc(-c3ccccc3)nc(-c3c(-c4cc5ccccc5c5ccccc45)ccc4oc5ccccc5c34)n2)cc1 ; c1ccc(-c2nc(-c3ccccc3)nc(-c3cccc4oc5c(-c6cc7ccccc7c7ccccc67)cccc5c34)n2)cc1]

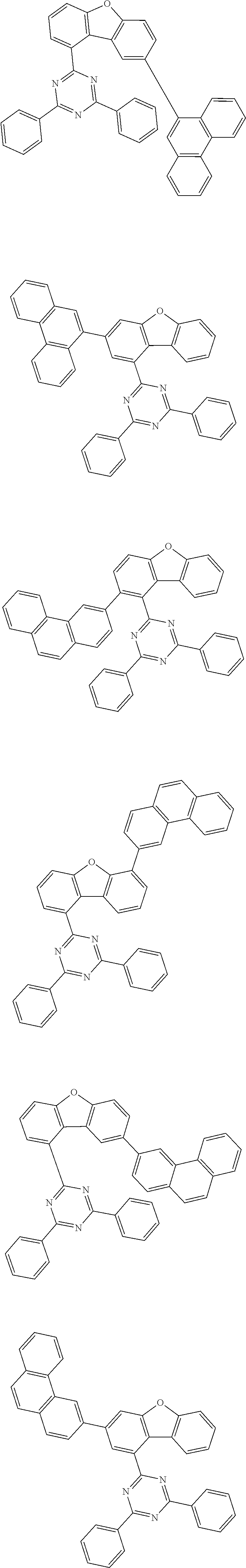 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3c(-c4ccc5ccc6ccccc6c5c4)ccc4oc5ccccc5c34)n2)cc1 ; c1ccc(-c2nc(-c3ccccc3)nc(-c3cccc4oc5c(-c6ccc7ccc8ccccc8c7c6)cccc5c34)n2)cc1 ; c1ccc(-c2nc(-c3ccccc3)nc(-c3cccc4oc5ccc(-c6ccc7ccc8ccccc8c7c6)cc5c34)n2)cc1 ; c1ccc(-c2nc(-c3ccccc3)nc(-c3cc(-c4ccc5ccc6ccccc6c5c4)cc4oc5ccccc5c34)n2)cc1]

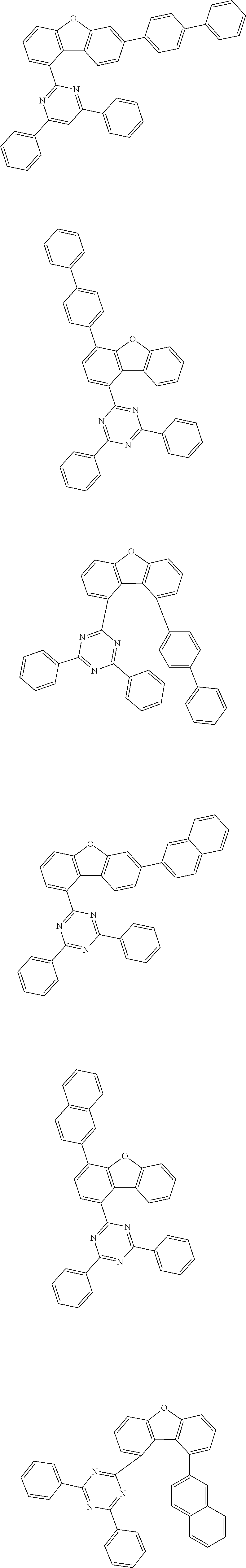 [SMILES: c1ccc(-c2ccc(-c3ccc4c(c3)oc3cccc(-c5nc(-c6ccccc6)cc(-c6ccccc6)n5)c34)cc2)cc1 ; c1ccc(-c2ccc(-c3ccc(-c4nc(-c5ccccc5)nc(-c5ccccc5)n4)c4c3oc3ccccc34)cc2)cc1 ; c1ccc(-c2ccc(-c3cccc4oc5cccc(-c6nc(-c7ccccc7)nc(-c7ccccc7)n6)c5c34)cc2)cc1 ; c1ccc(-c2nc(-c3ccccc3)nc(-c3cccc4oc5cc(-c6ccc7ccccc7c6)ccc5c34)n2)cc1 ; c1ccc(-c2nc(-c3ccccc3)nc(-c3ccc(-c4ccc5ccccc5c4)c4oc5ccccc5c34)n2)cc1 ; c1ccc(-c2nc(-c3ccccc3)nc(-c3cccc4oc5cccc(-c6ccc7ccccc7c6)c5c34)n2)cc1]

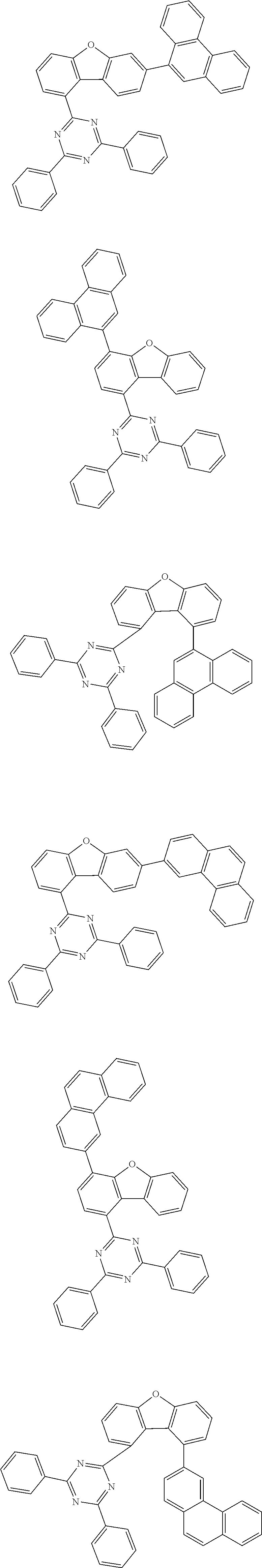 [SMILES: c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1 ; c1ccc(-c2nc(-c3ccccc3)nc(-c3cccc4oc5cccc(-c6cc7ccccc7c7ccccc67)c5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

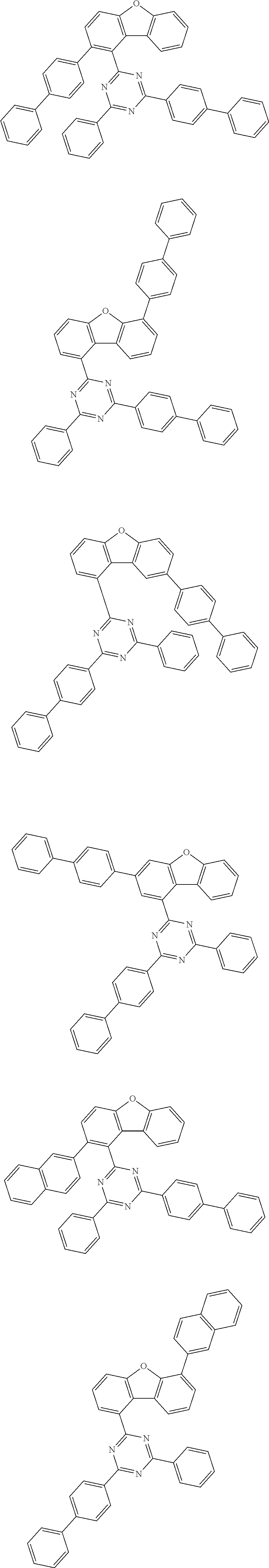

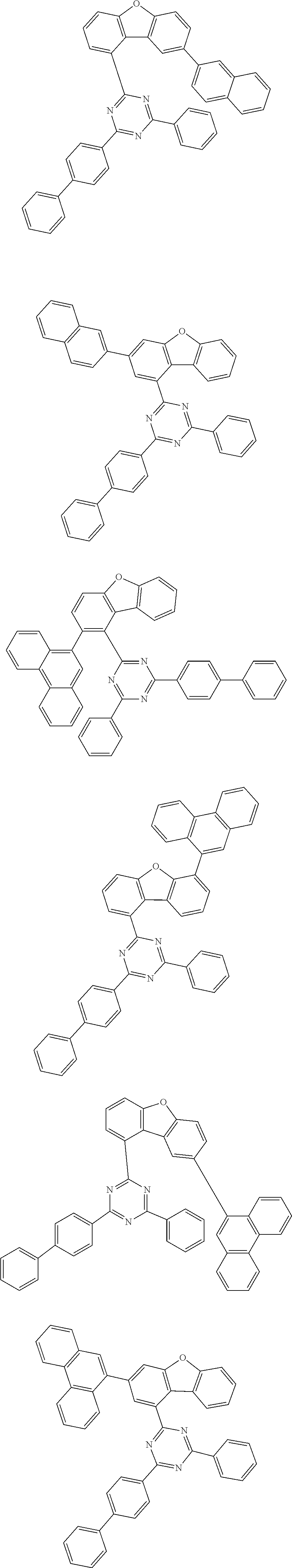

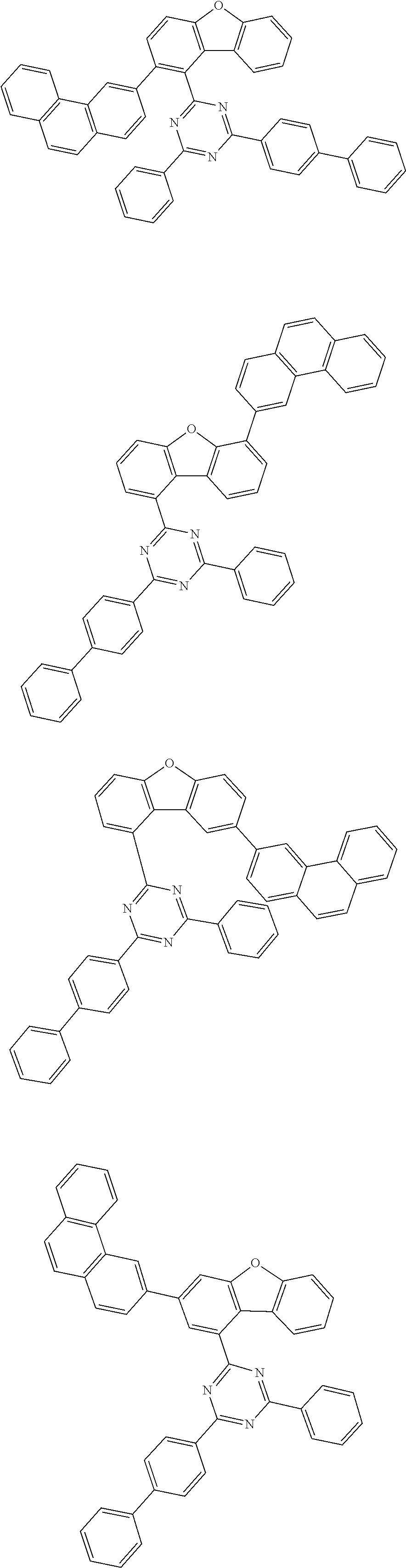 [SMILES: c1ccc(-c2ccc(-c3nc(-c4ccccc4)nc(-c4c(-c5ccc6ccc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2ccc(-c3nc(-c4ccccc4)nc(-c4cccc5oc6ccc(-c7ccc8ccc9ccccc9c8c7)cc6c45)n3)cc2)cc1 ; c1ccc(-c2ccc(-c3nc(-c4ccccc4)nc(-c4cc(-c5ccc6ccc7ccccc7c6c5)cc5oc6ccccc6c45)n3)cc2)cc1]

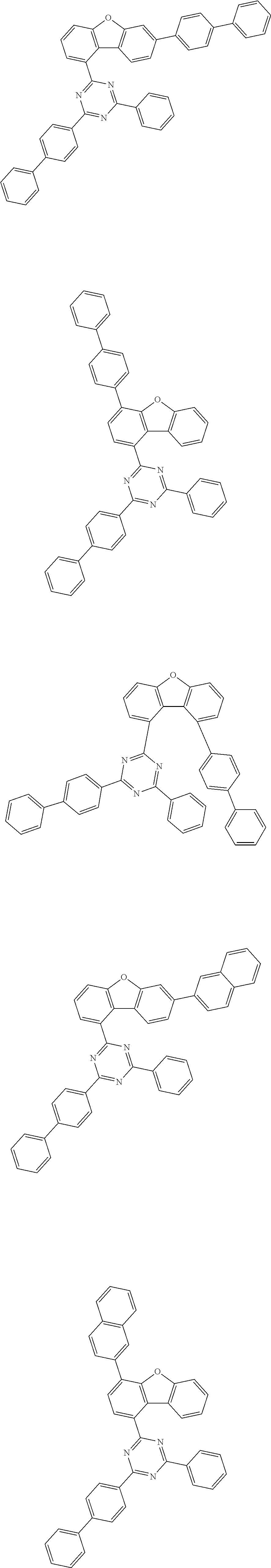 [SMILES: c1ccc(-c2ccc(-c3ccc4c(c3)oc3cccc(-c5nc(-c6ccccc6)nc(-c6ccc(-c7ccccc7)cc6)n5)c34)cc2)cc1 ; c1ccc(-c2ccc(-c3nc(-c4ccccc4)nc(-c4ccc(-c5ccc(-c6ccccc6)cc5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1]

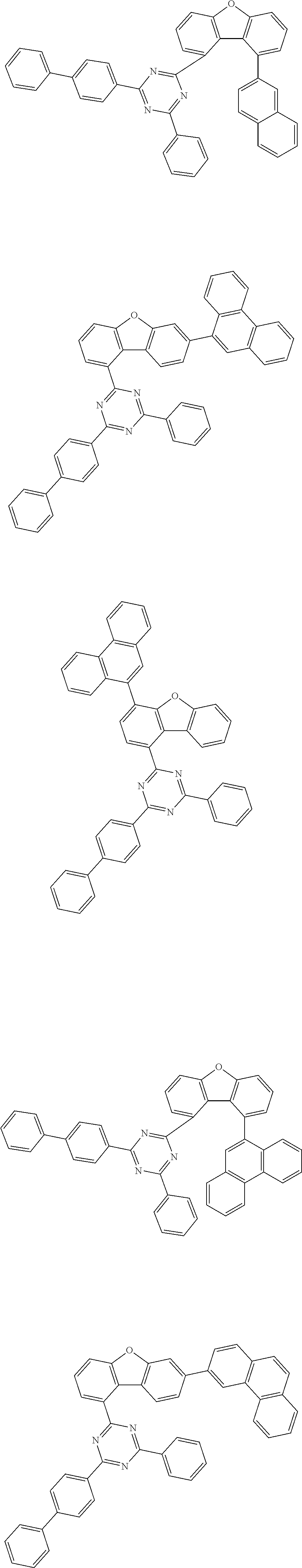 [SMILES: c1ccc(-c2ccc(-c3nc(-c4ccccc4)nc(-c4cccc5oc6cccc(-c7ccc8ccccc8c7)c6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1 ; c1ccc(-c2ccc(-c3nc(-c4ccccc4)nc(-c4cccc5oc6cccc(-c7cc8ccccc8c8ccccc78)c6c45)n3)cc2)cc1 ; c1ccc(-c2ccc(-c3nc(-c4ccccc4)nc(-c4cccc5oc6cc(-c7ccc8ccc9ccccc9c8c7)ccc6c45)n3)cc2)cc1]

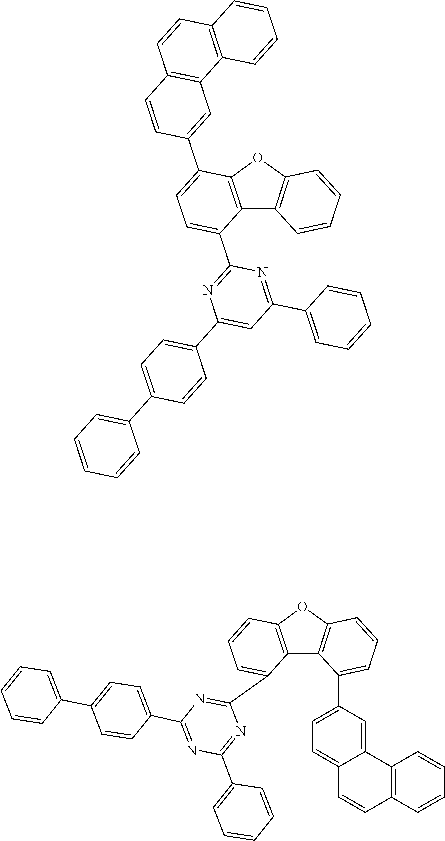 [SMILES: c1ccc(-c2ccc(-c3cc(-c4ccccc4)nc(-c4ccc(-c5ccc6ccc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8ccc9ccccc9c8c7)c6c45)n3)cc2)cc1]

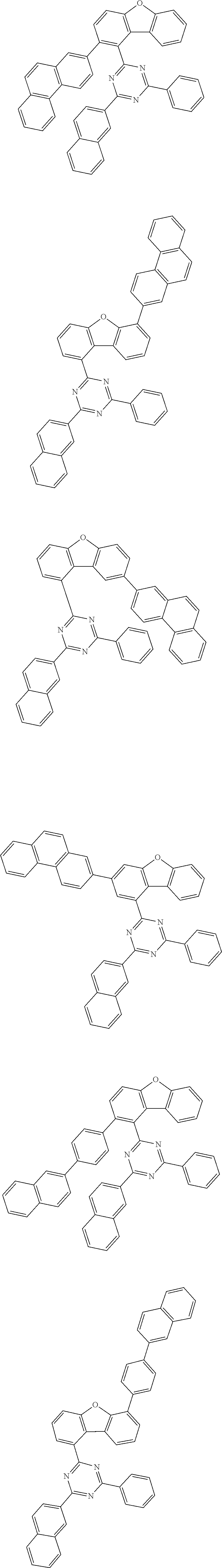

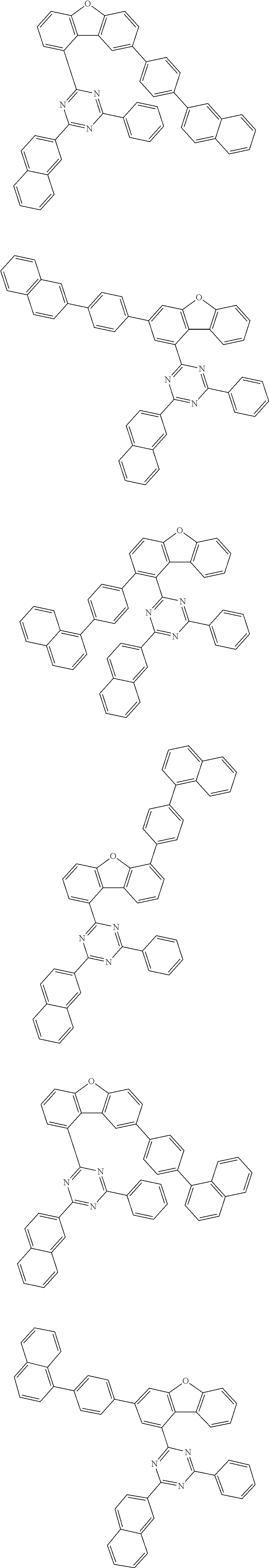

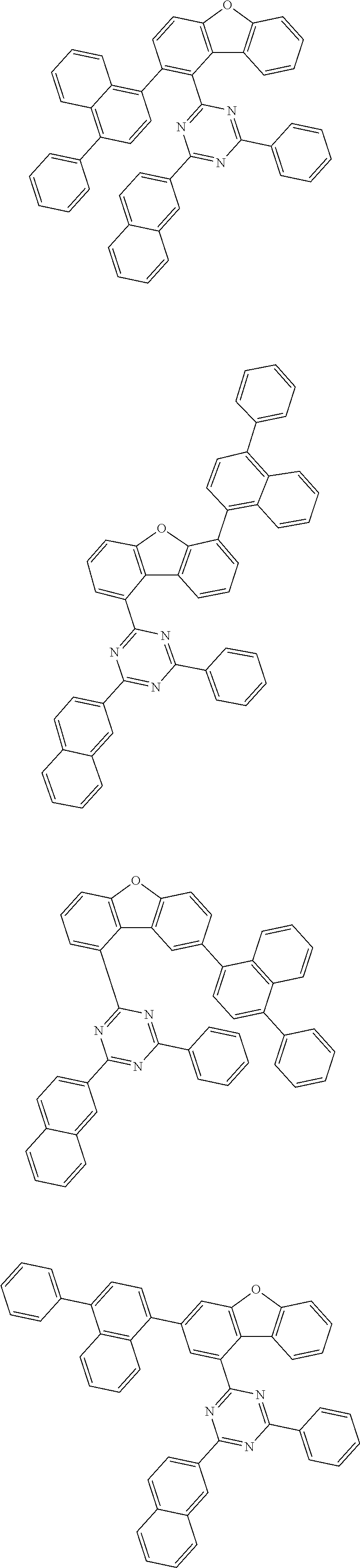 [SMILES: c1ccc(-c2nc(-c3ccc4ccccc4c3)nc(-c3c(-c4ccc(-c5ccccc5)c5ccccc45)ccc4oc5ccccc5c34)n2)cc1 ; c1ccc(-c2nc(-c3ccc4ccccc4c3)nc(-c3cccc4oc5c(-c6ccc(-c7ccccc7)c7ccccc67)cccc5c34)n2)cc1 ; c1ccc(-c2nc(-c3ccc4ccccc4c3)nc(-c3cccc4oc5ccc(-c6ccc(-c7ccccc7)c7ccccc67)cc5c34)n2)cc1 ; c1ccc(-c2nc(-c3ccc4ccccc4c3)nc(-c3cc(-c4ccc(-c5ccccc5)c5ccccc45)cc4oc5ccccc5c34)n2)cc1]

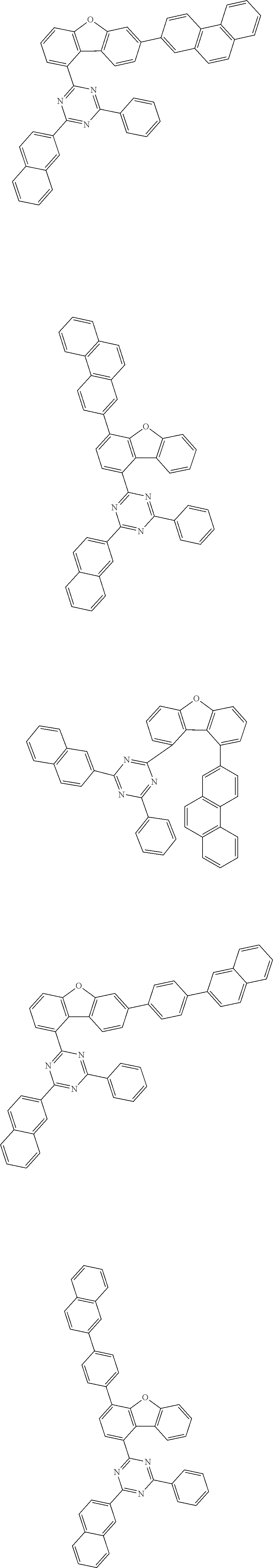 [SMILES: c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1 ; c1ccc(-c2nc(-c3ccc4ccccc4c3)nc(-c3cccc4oc5cccc(-c6ccc7c(ccc8ccccc87)c6)c5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1]

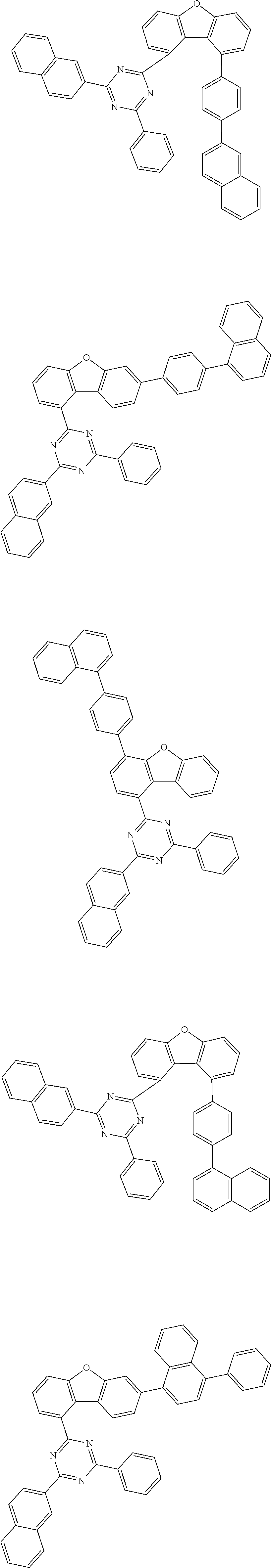 [SMILES: c1ccc(-c2nc(-c3ccc4ccccc4c3)nc(-c3cccc4oc5cccc(-c6ccc(-c7ccc8ccccc8c7)cc6)c5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1 ; c1ccc(-c2nc(-c3ccc4ccccc4c3)nc(-c3ccc(-c4ccc(-c5cccc6ccccc56)cc4)c4oc5ccccc5c34)n2)cc1 ; c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3cccc4oc5cc(-c6ccc(-c7ccccc7)c7ccccc67)ccc5c34)n2)cc1]

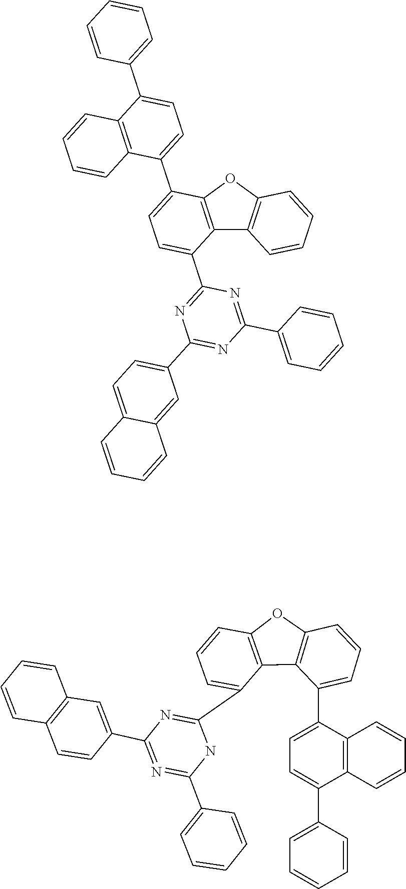

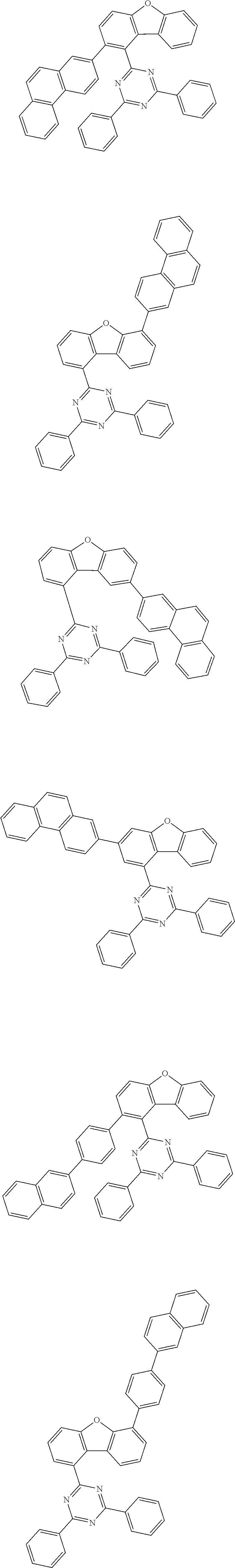 [SMILES: c1ccc(-c2nc(-c3ccccc3)nc(-c3c(-c4ccc5c(ccc6ccccc65)c4)ccc4oc5ccccc5c34)n2)cc1 ; c1ccc(-c2nc(-c3ccccc3)nc(-c3cccc4oc5c(-c6ccc7c(ccc8ccccc87)c6)cccc5c34)n2)cc1 ; c1ccc(-c2nc(-c3ccccc3)nc(-c3cccc4oc5ccc(-c6ccc7c(ccc8ccccc87)c6)cc5c34)n2)cc1 ; c1ccc(-c2nc(-c3ccccc3)nc(-c3cc(-c4ccc5c(ccc6ccccc65)c4)cc4oc5ccccc5c34)n2)cc1 ; c1ccc(-c2nc(-c3ccccc3)nc(-c3c(-c4ccc(-c5ccc6ccccc6c5)cc4)ccc4oc5ccccc5c34)n2)cc1 ; c1ccc(-c2nc(-c3ccccc3)nc(-c3cccc4oc5c(-c6ccc(-c7ccc8ccccc8c7)cc6)cccc5c34)n2)cc1]

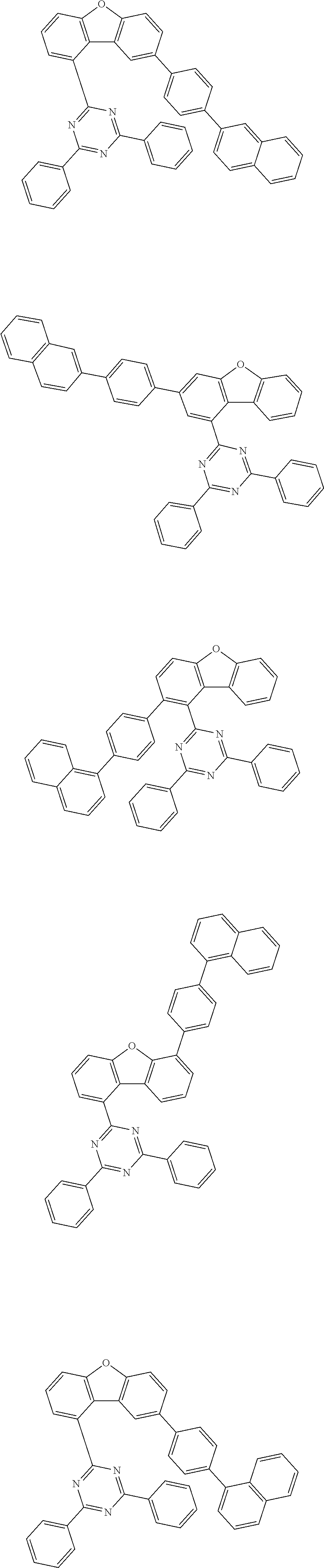 [SMILES: c1ccc(-c2nc(-c3ccccc3)nc(-c3cccc4oc5ccc(-c6ccc(-c7ccc8ccccc8c7)cc6)cc5c34)n2)cc1 ; c1ccc(-c2nc(-c3ccccc3)nc(-c3cc(-c4ccc(-c5ccc6ccccc6c5)cc4)cc4oc5ccccc5c34)n2)cc1 ; c1ccc(-c2nc(-c3ccccc3)nc(-c3c(-c4ccc(-c5cccc6ccccc56)cc4)ccc4oc5ccccc5c34)n2)cc1 ; c1ccc(-c2nc(-c3ccccc3)nc(-c3cccc4oc5c(-c6ccc(-c7cccc8ccccc78)cc6)cccc5c34)n2)cc1 ; c1ccc(-c2nc(-c3ccccc3)nc(-c3cccc4oc5ccc(-c6ccc(-c7cccc8ccccc78)cc6)cc5c34)n2)cc1]

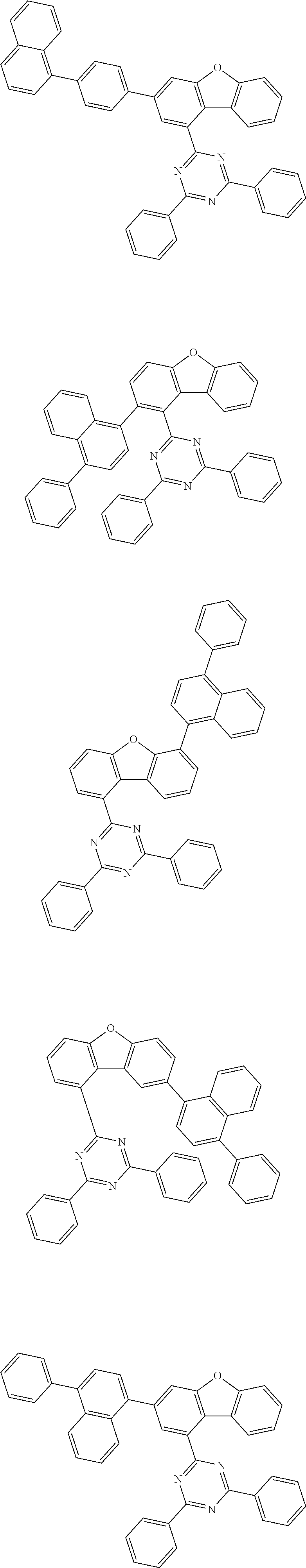 [SMILES: c1ccc(-c2nc(-c3ccccc3)nc(-c3cc(-c4ccc(-c5cccc6ccccc56)cc4)cc4oc5ccccc5c34)n2)cc1 ; c1ccc(-c2nc(-c3ccccc3)nc(-c3c(-c4ccc(-c5ccccc5)c5ccccc45)ccc4oc5ccccc5c34)n2)cc1 ; c1ccc(-c2nc(-c3ccccc3)nc(-c3cccc4oc5c(-c6ccc(-c7ccccc7)c7ccccc67)cccc5c34)n2)cc1 ; c1ccc(-c2nc(-c3ccccc3)nc(-c3cccc4oc5ccc(-c6ccc(-c7ccccc7)c7ccccc67)cc5c34)n2)cc1 ; c1ccc(-c2nc(-c3ccccc3)nc(-c3cc(-c4ccc(-c5ccccc5)c5ccccc45)cc4oc5ccccc5c34)n2)cc1]

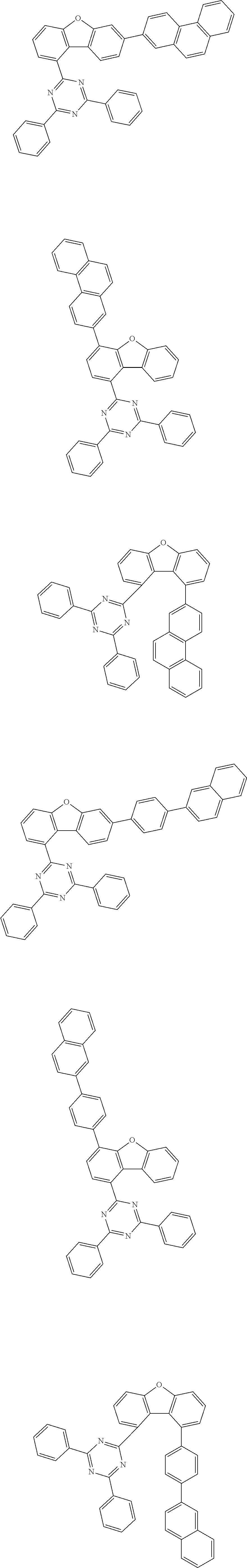 [SMILES: c1ccc(-c2nc(-c3ccccc3)nc(-c3cccc4oc5cc(-c6ccc7c(ccc8ccccc87)c6)ccc5c34)n2)cc1 ; c1ccc(-c2nc(-c3ccccc3)nc(-c3ccc(-c4ccc5c(ccc6ccccc65)c4)c4oc5ccccc5c34)n2)cc1 ; c1ccc(-c2nc(-c3ccccc3)nc(-c3cccc4oc5cccc(-c6ccc7c(ccc8ccccc87)c6)c5c34)n2)cc1 ; c1ccc(-c2nc(-c3ccccc3)nc(-c3cccc4oc5cc(-c6ccc(-c7ccc8ccccc8c7)cc6)ccc5c34)n2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1]

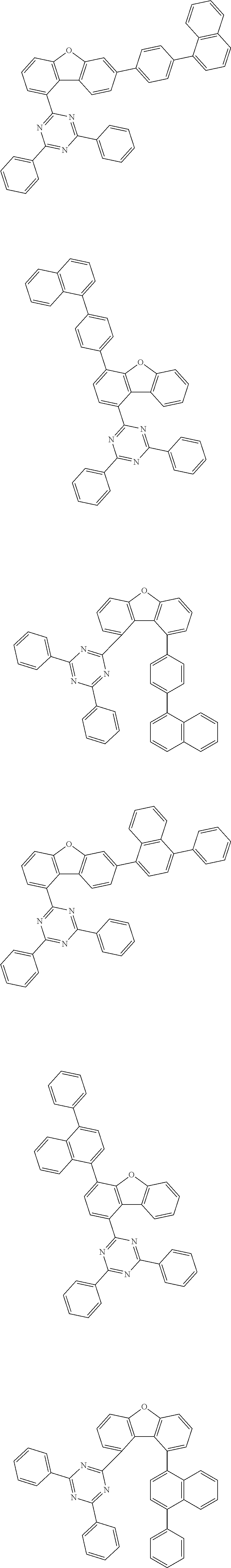 [SMILES: c1ccc(-c2nc(-c3ccccc3)nc(-c3cccc4oc5cc(-c6ccc(-c7cccc8ccccc78)cc6)ccc5c34)n2)cc1 ; c1ccc(-c2nc(-c3ccccc3)nc(-c3ccc(-c4ccc(-c5cccc6ccccc56)cc4)c4oc5ccccc5c34)n2)cc1 ; c1ccc(-c2nc(-c3ccccc3)nc(-c3cccc4oc5cccc(-c6ccc(-c7cccc8ccccc78)cc6)c5c34)n2)cc1 ; c1ccc(-c2nc(-c3ccccc3)nc(-c3cccc4oc5cc(-c6ccc(-c7ccccc7)c7ccccc67)ccc5c34)n2)cc1 ; c1ccc(-c2nc(-c3ccccc3)nc(-c3ccc(-c4ccc(-c5ccccc5)c5ccccc45)c4oc5ccccc5c34)n2)cc1 ; c1ccc(-c2nc(-c3ccccc3)nc(-c3cccc4oc5cccc(-c6ccc(-c7ccccc7)c7ccccc67)c5c34)n2)cc1]

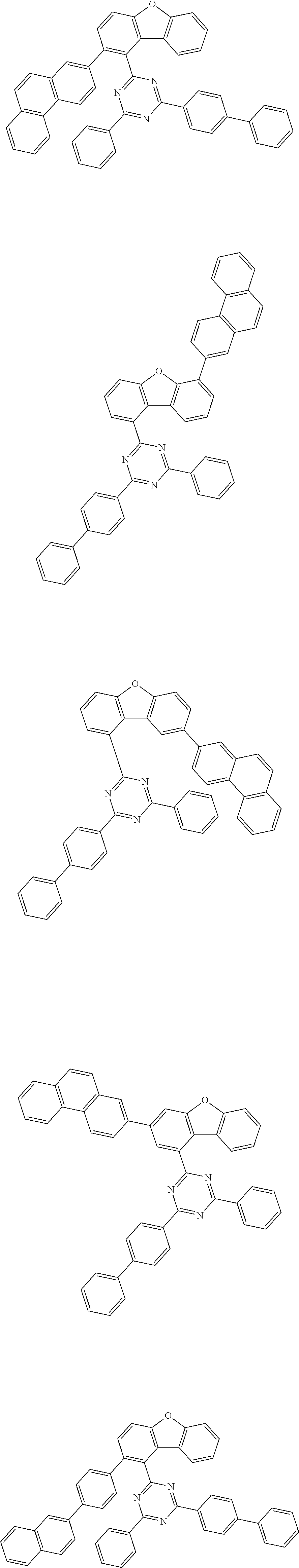 [SMILES: c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1 ; c1ccc(-c2ccc(-c3nc(-c4ccccc4)nc(-c4cccc5oc6ccc(-c7ccc8c(ccc9ccccc98)c7)cc6c45)n3)cc2)cc1 ; c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1]

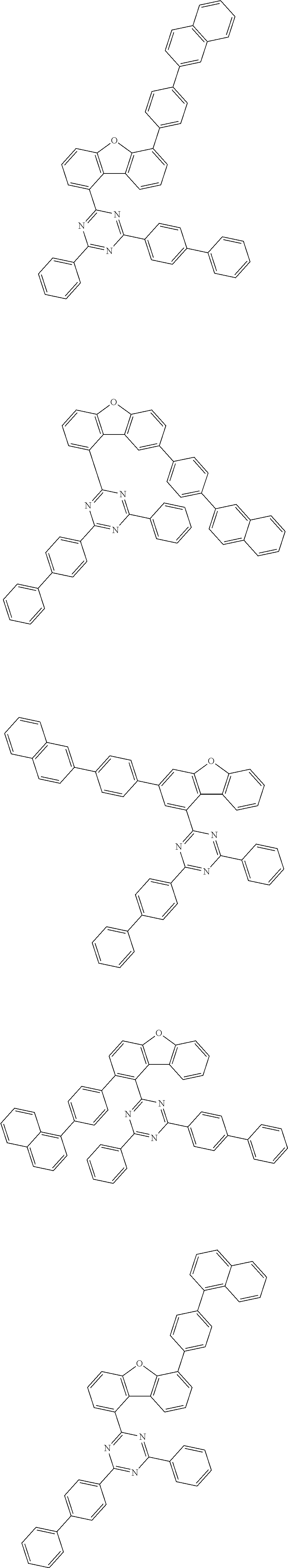 [SMILES: c1ccc(-c2ccc(-c3nc(-c4ccccc4)nc(-c4cccc5oc6c(-c7ccc(-c8ccc9ccccc9c8)cc7)cccc6c45)n3)cc2)cc1 ; c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1 ; c1ccc(-c2ccc(-c3nc(-c4ccccc4)nc(-c4c(-c5ccc(-c6cccc7ccccc67)cc5)ccc5oc6ccccc6c45)n3)cc2)cc1 ; c1ccc(-c2ccc(-c3nc(-c4ccccc4)nc(-c4cccc5oc6c(-c7ccc(-c8cccc9ccccc89)cc7)cccc6c45)n3)cc2)cc1]

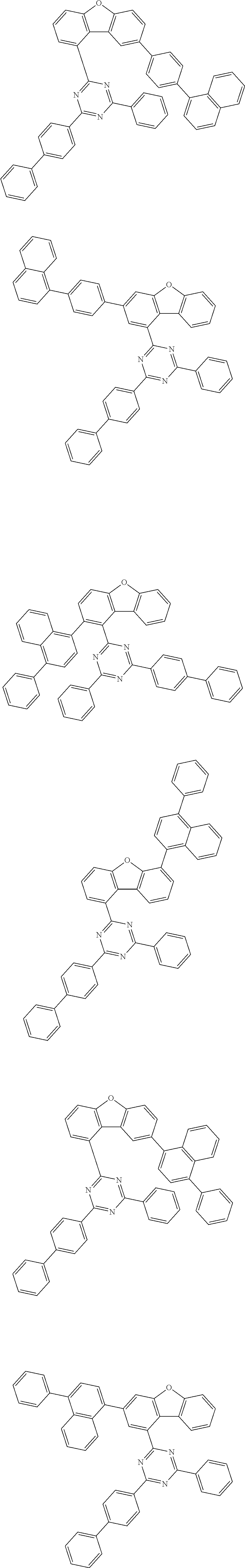

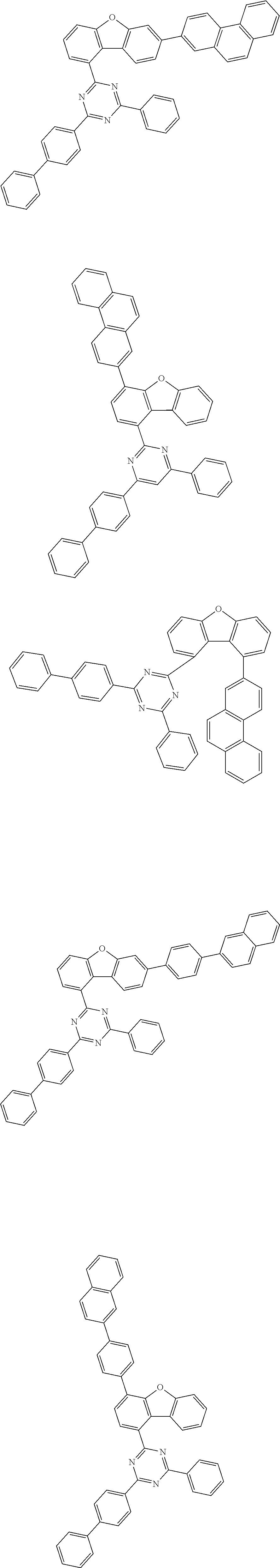 [SMILES: c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3cc(-c4ccccc4)nc(-c4ccc(-c5ccc6c(ccc7ccccc76)c5)c5oc6ccccc6c45)n3)cc2)cc1 ; c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2ccc(-c3nc(-c4ccccc4)nc(-c4cccc5oc6cc(-c7ccc(-c8ccc9ccccc9c8)cc7)ccc6c45)n3)cc2)cc1 ; c1ccc(-c2ccc(-c3nc(-c4ccccc4)nc(-c4ccc(-c5ccc(-c6ccc7ccccc7c6)cc5)c5oc6ccccc6c45)n3)cc2)cc1]

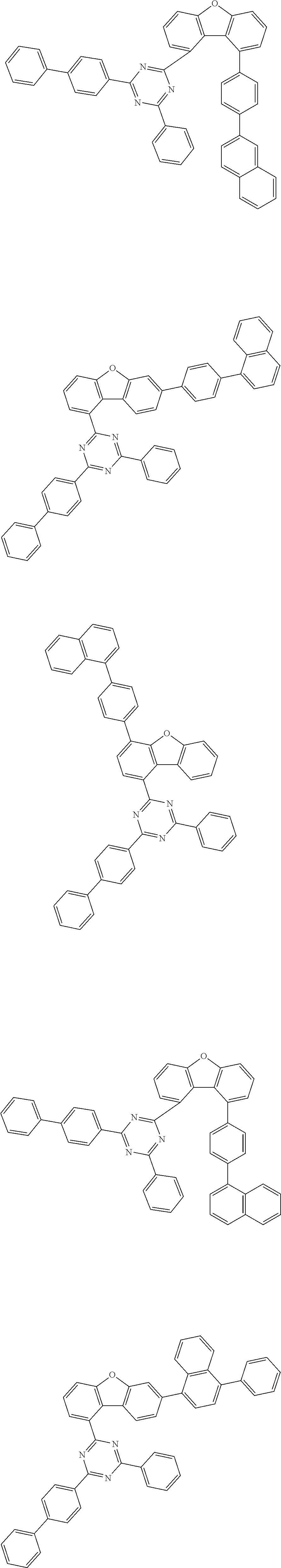 [SMILES: c1ccc(-c2ccc(-c3nc(-c4ccccc4)nc(-c4cccc5oc6cccc(-c7ccc(-c8ccc9ccccc9c8)cc7)c6c45)n3)cc2)cc1 ; c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1 ; c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2ccc(-c3nc(-c4ccccc4)nc(-c4cccc5oc6cc(-c7ccc(-c8ccccc8)c8ccccc78)ccc6c45)n3)cc2)cc1]

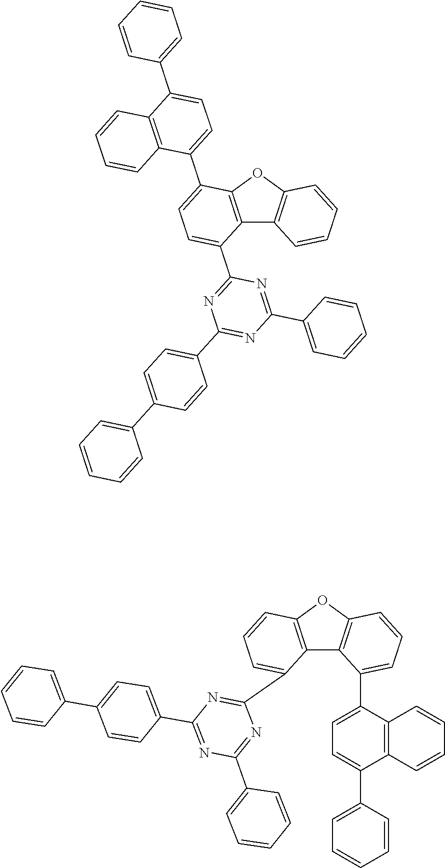 [SMILES: c1ccc(-c2ccc(-c3nc(-c4ccccc4)nc(-c4ccc(-c5ccc(-c6ccccc6)c6ccccc56)c5oc6ccccc6c45)n3)cc2)cc1 ; c1ccc(-c2ccc(-c3nc(-c4ccccc4)nc(-c4cccc5oc6cccc(-c7ccc(-c8ccccc8)c8ccccc78)c6c45)n3)cc2)cc1]

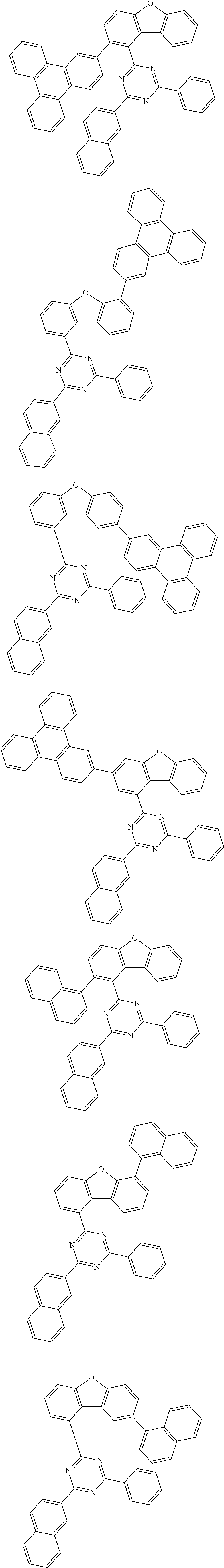

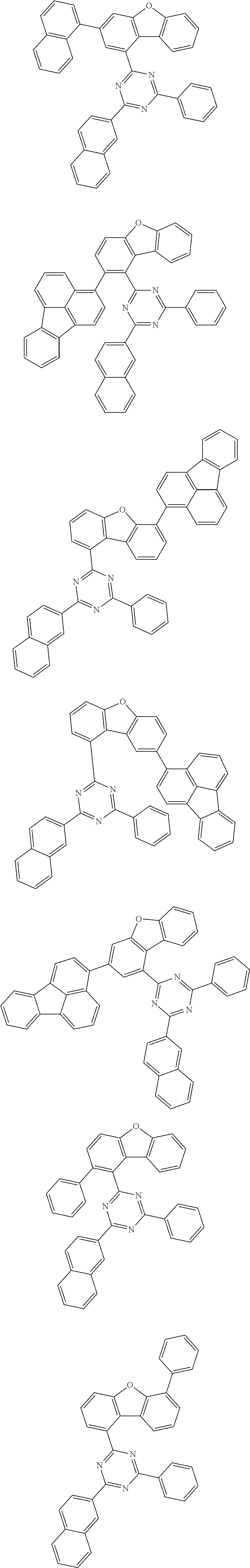

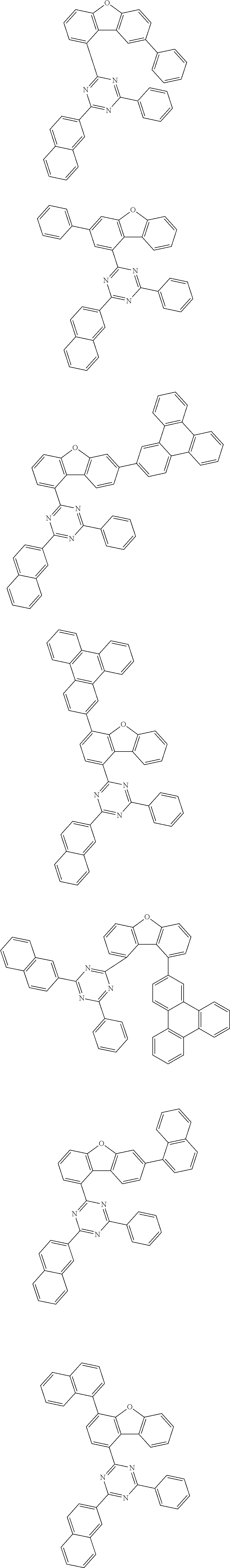

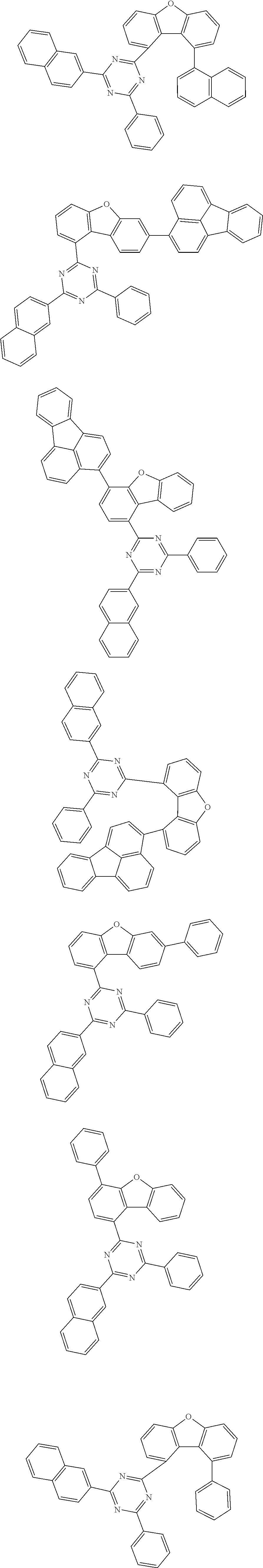

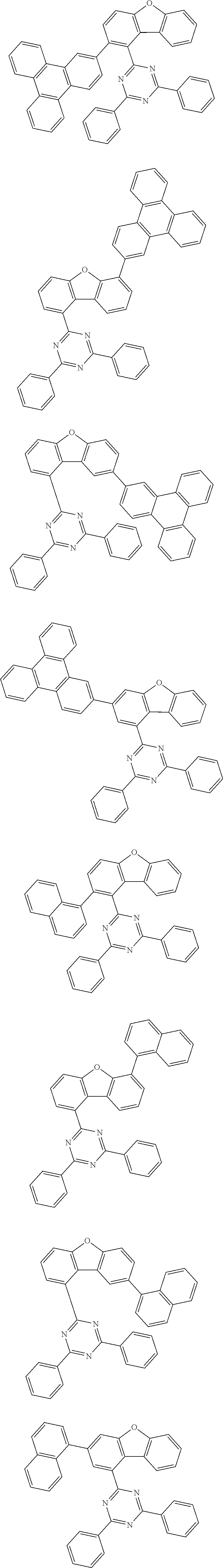

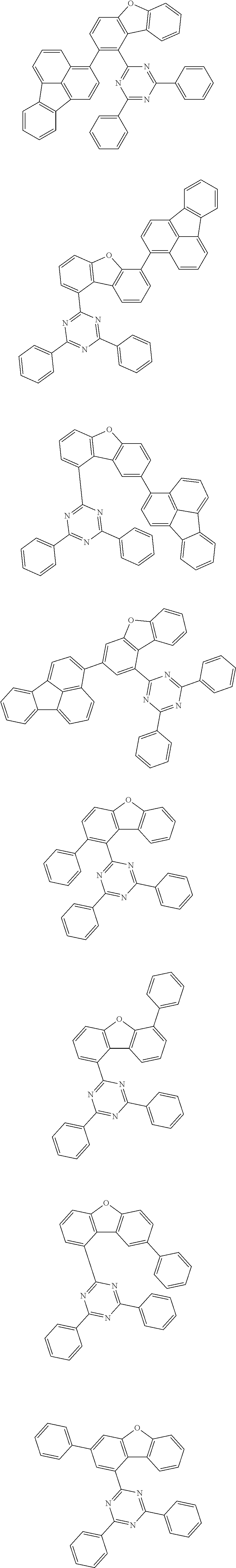

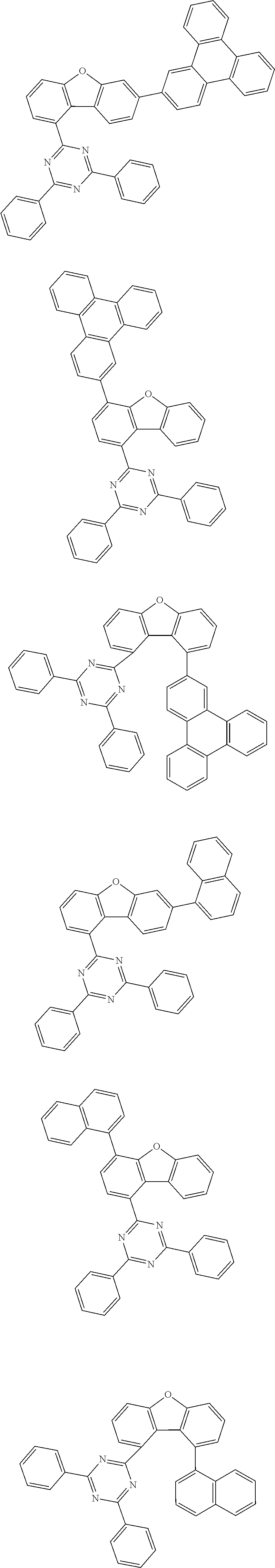 [SMILES: c1ccc(-c2nc(-c3ccccc3)nc(-c3cccc4oc5cc(-c6ccc7c8ccccc8c8ccccc8c7c6)ccc5c34)n2)cc1 ; c1ccc(-c2nc(-c3ccccc3)nc(-c3ccc(-c4ccc5c6ccccc6c6ccccc6c5c4)c4oc5ccccc5c34)n2)cc1 ; c1ccc(-c2nc(-c3ccccc3)nc(-c3cccc4oc5cccc(-c6ccc7c8ccccc8c8ccccc8c7c6)c5c34)n2)cc1 ; c1ccc(-c2nc(-c3ccccc3)nc(-c3cccc4oc5cc(-c6cccc7ccccc67)ccc5c34)n2)cc1 ; c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1]

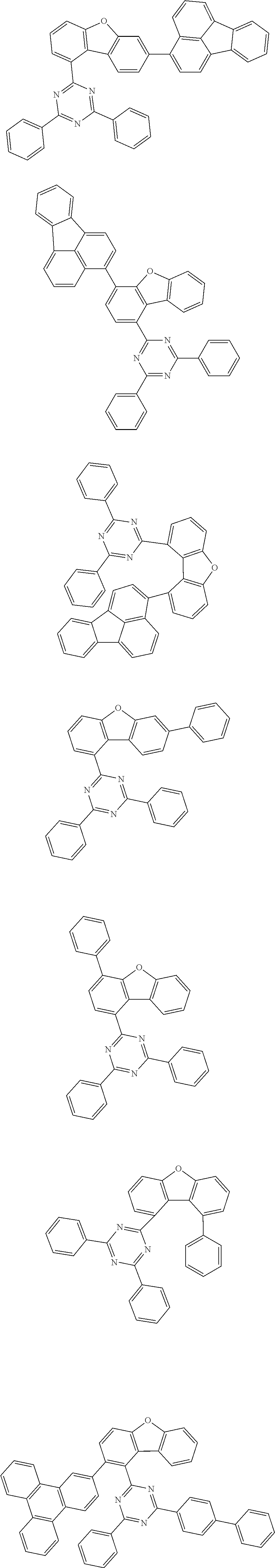

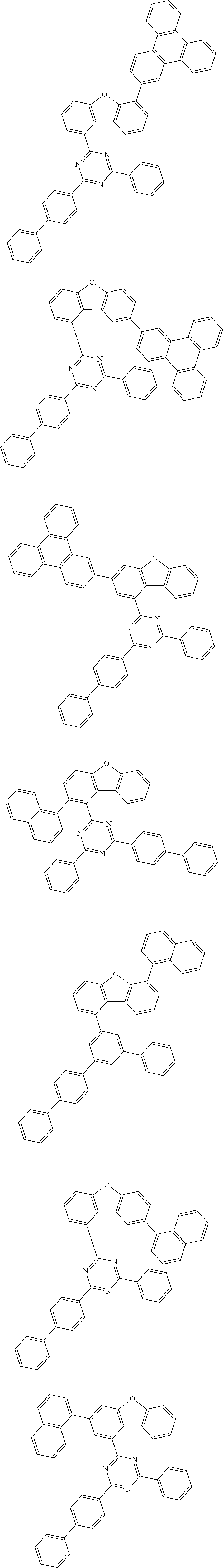

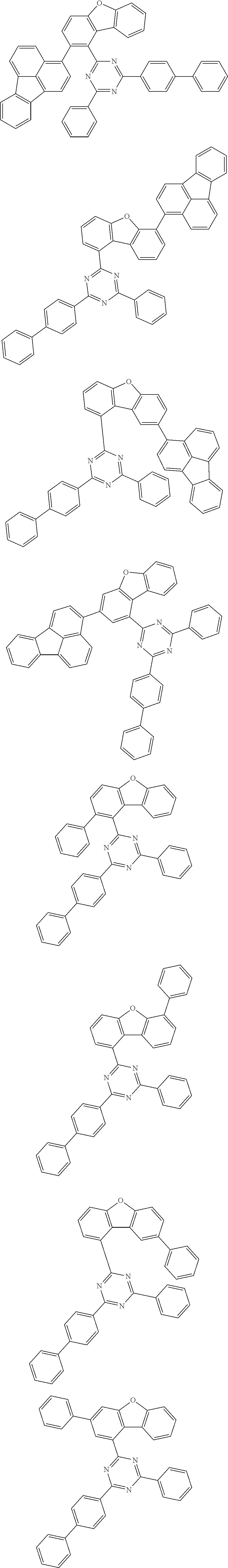

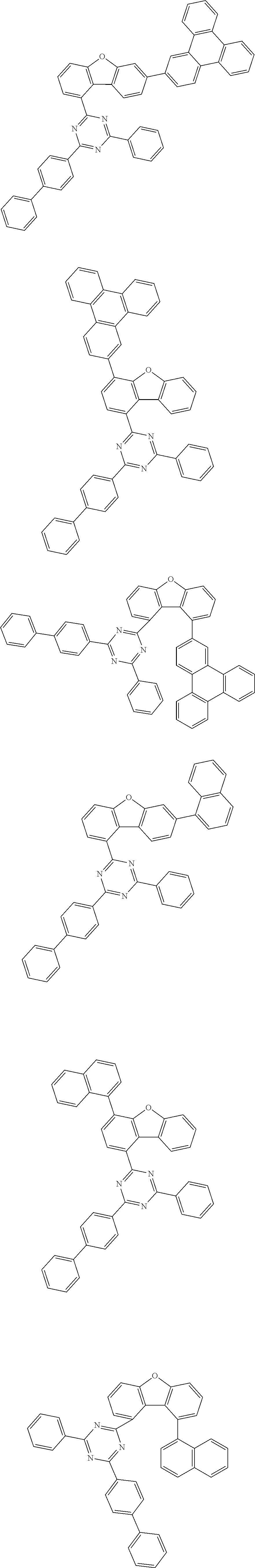

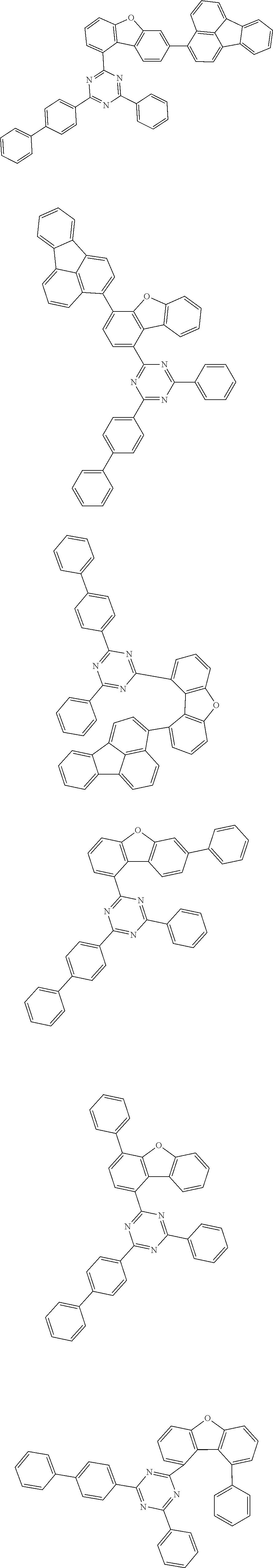

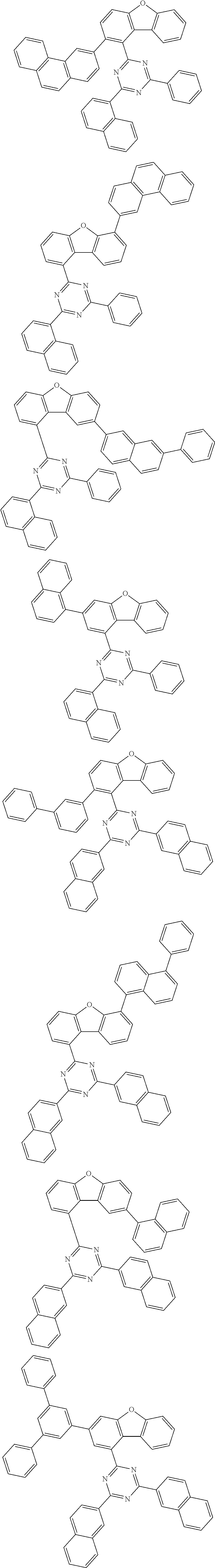

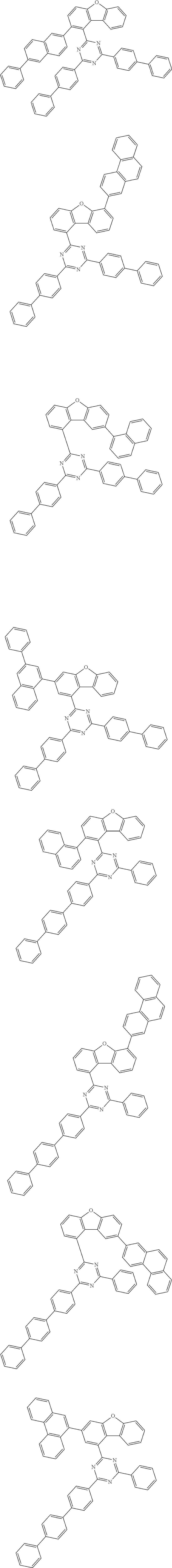

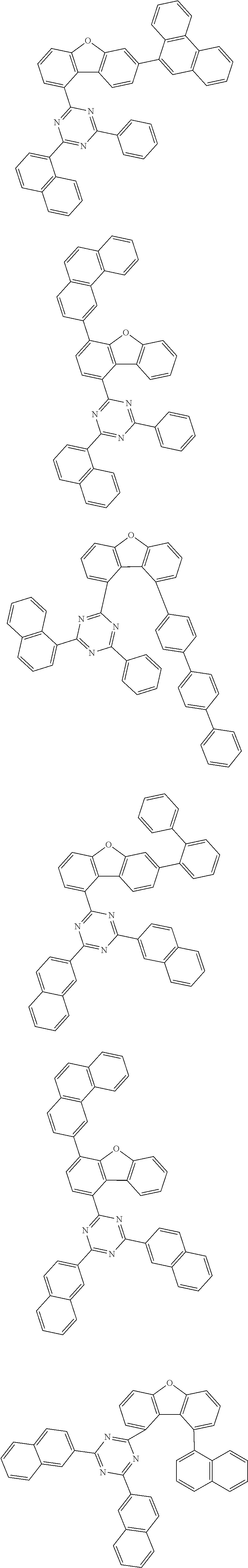

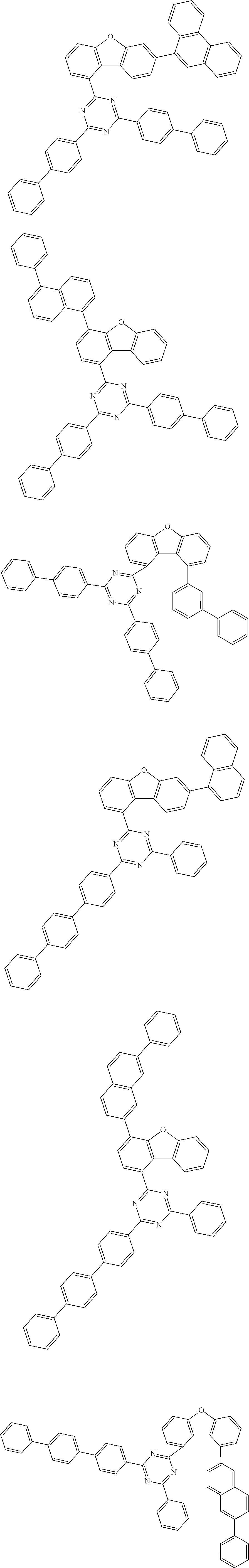

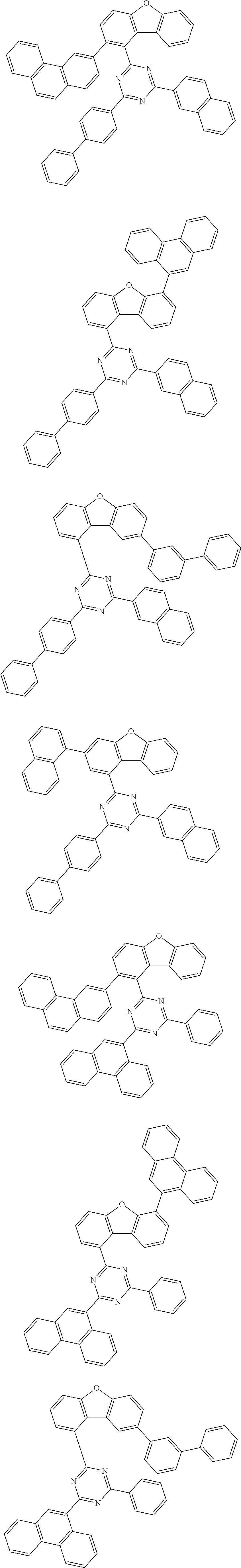

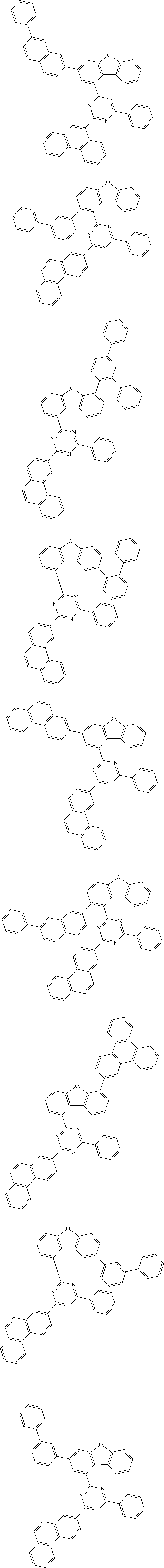

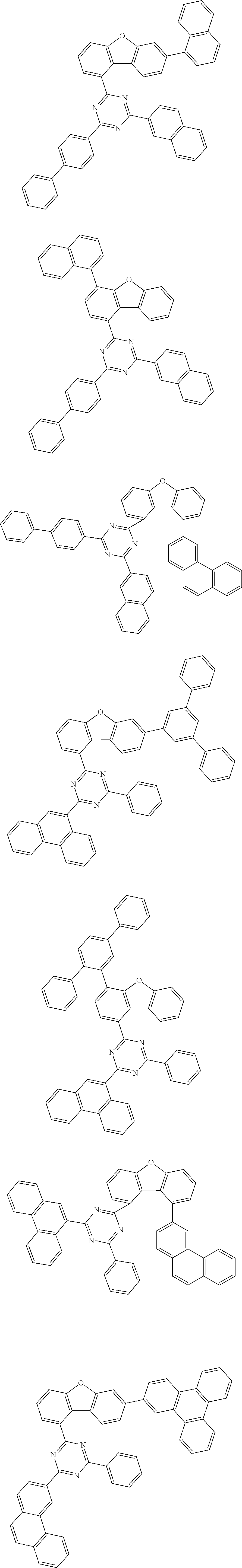

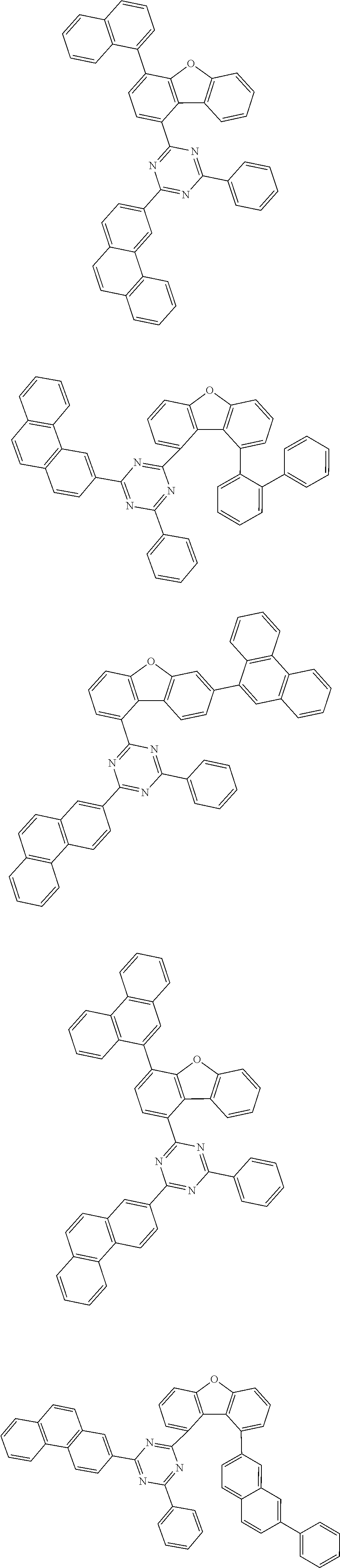 [SMILES: c1ccc(-c2nc(-c3ccc4ccc5ccccc5c4c3)nc(-c3ccc(-c4cccc5ccccc45)c4oc5ccccc5c34)n2)cc1 ; c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1 ; c1ccc(-c2nc(-c3ccc4c(ccc5ccccc54)c3)nc(-c3ccc(-c4cc5ccccc5c5ccccc45)c4oc5ccccc5c34)n2)cc1 ; c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1]

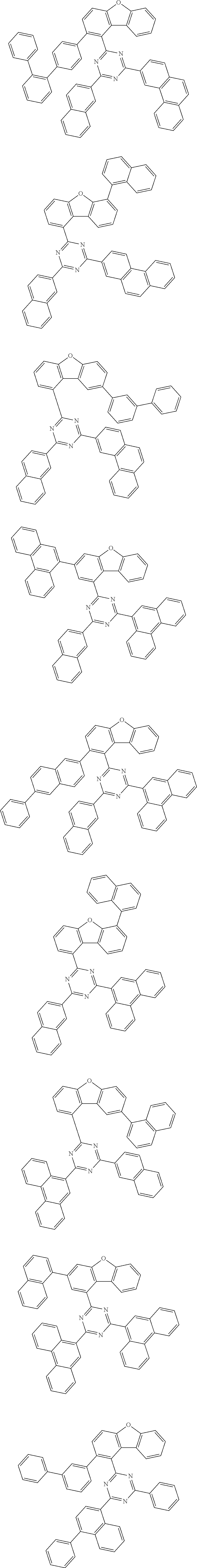

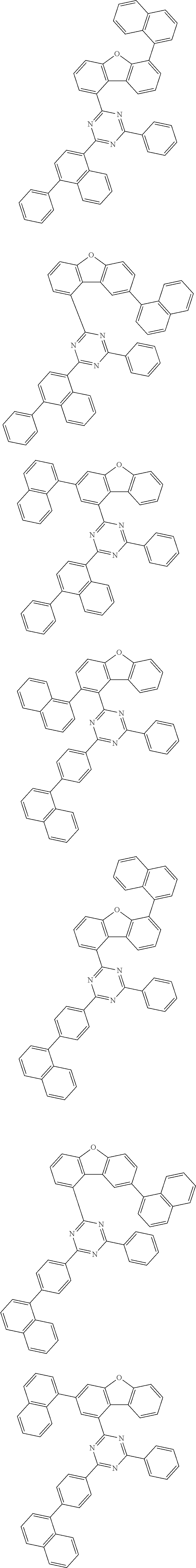

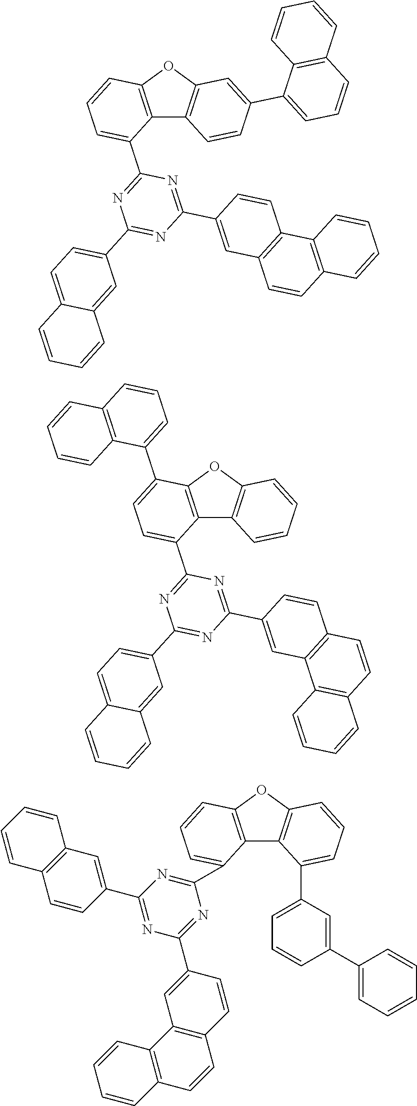

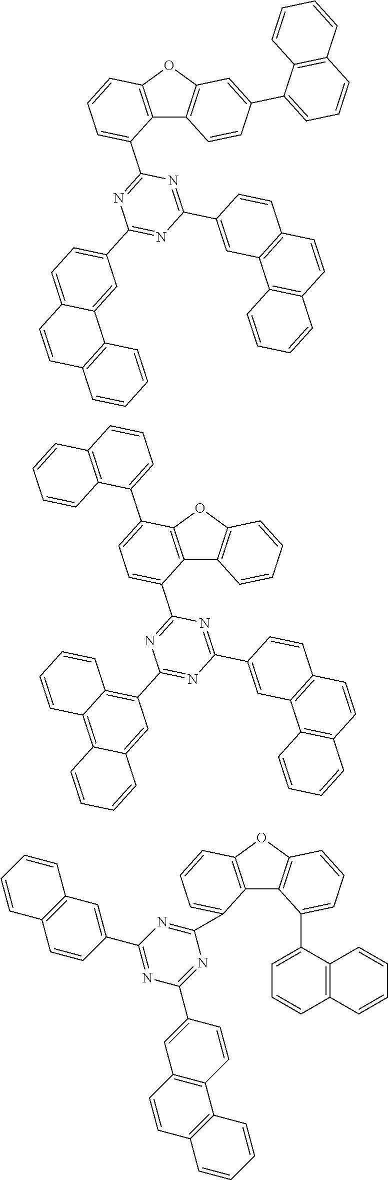

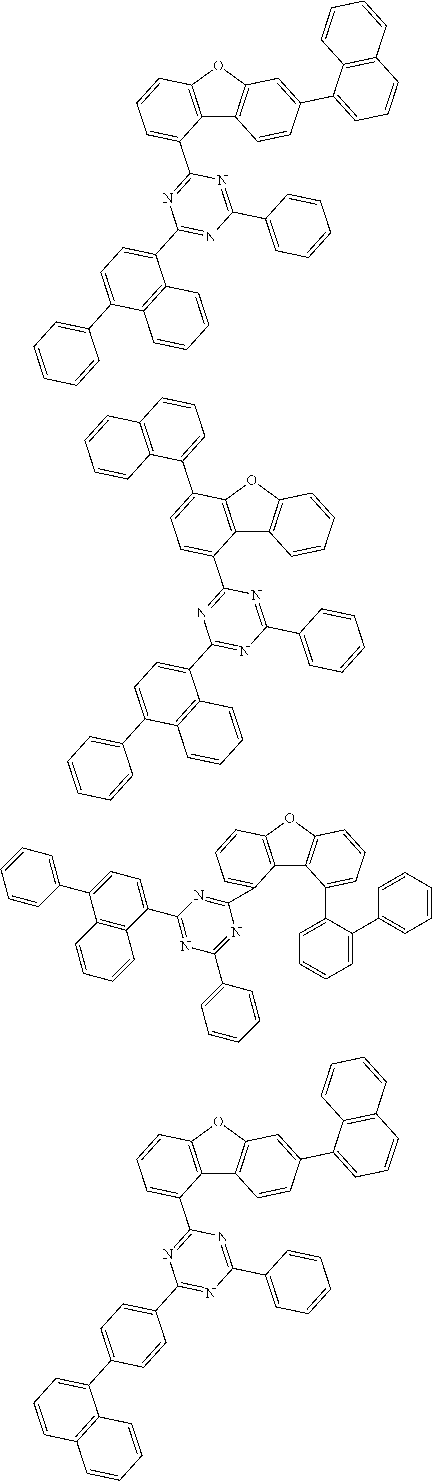

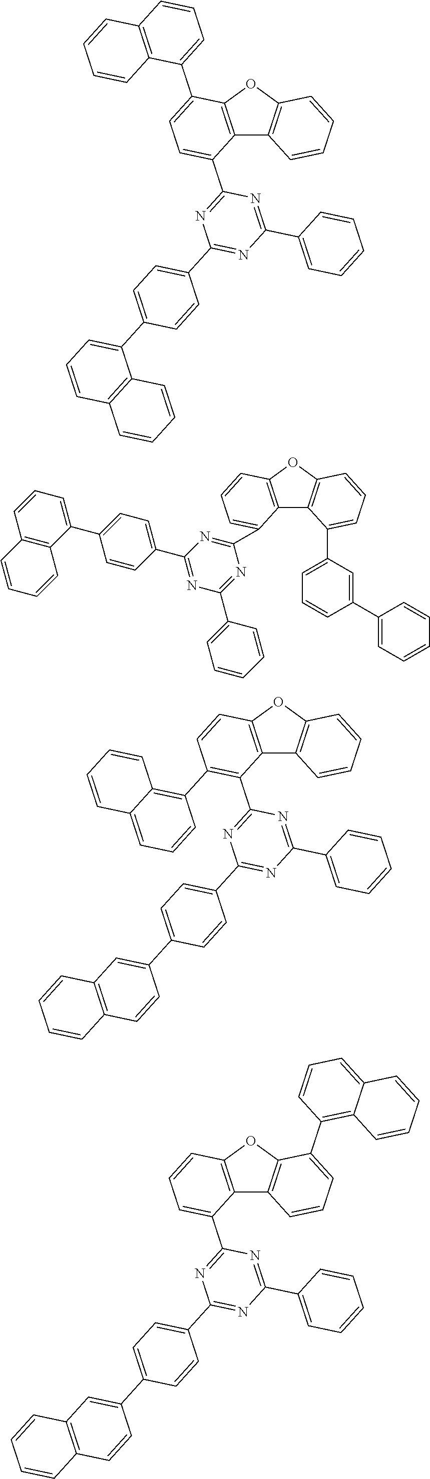

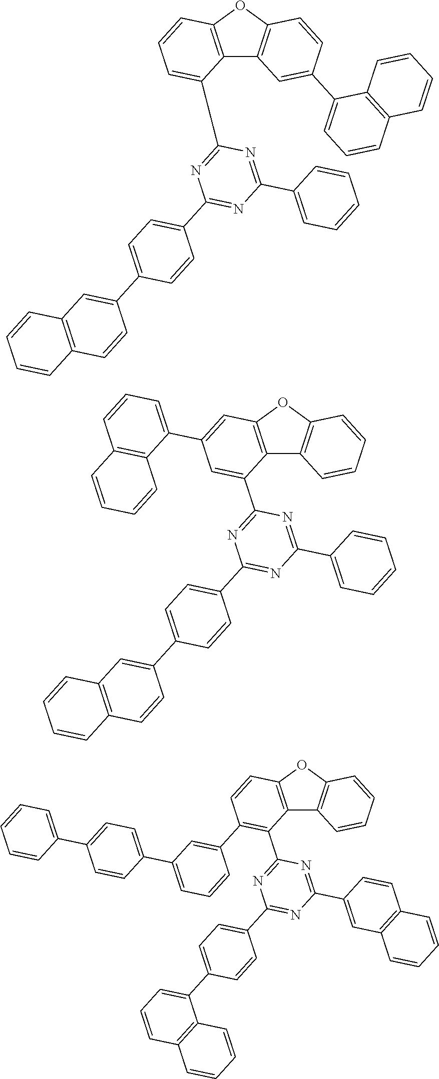

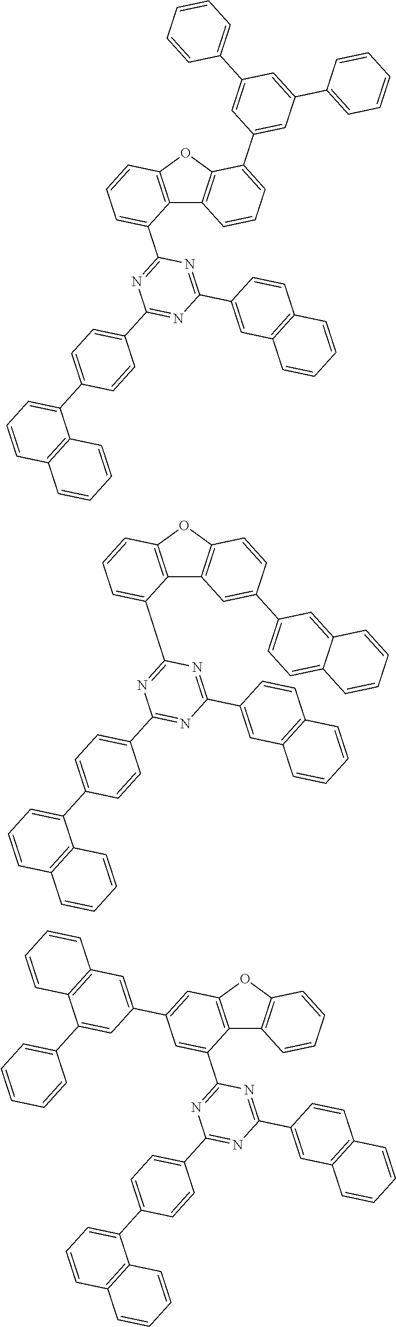

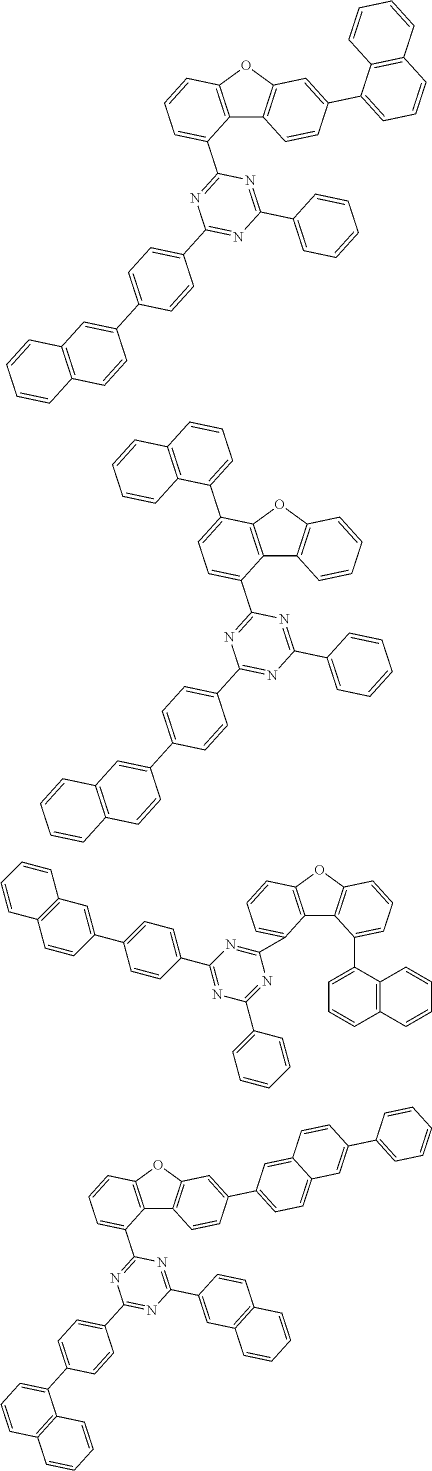

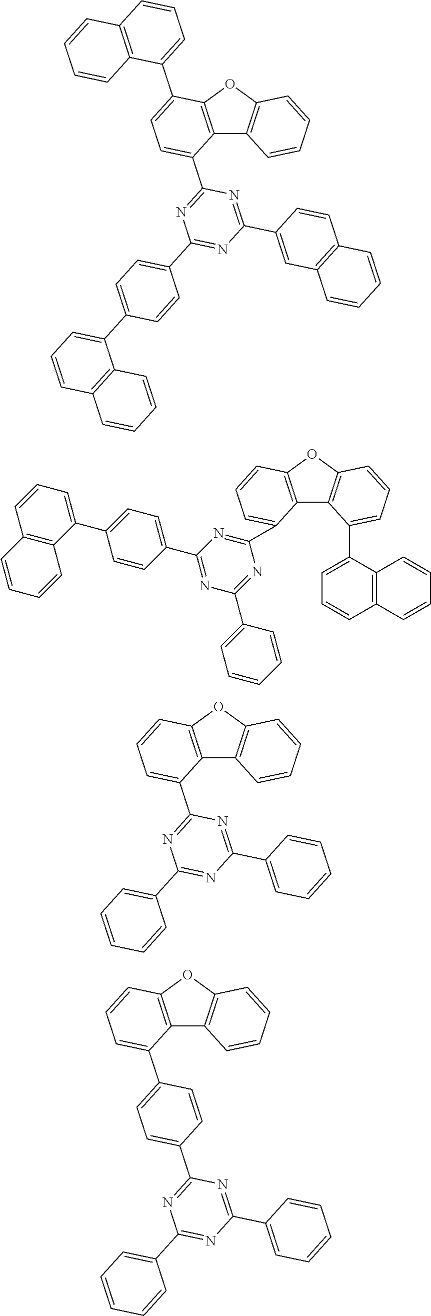

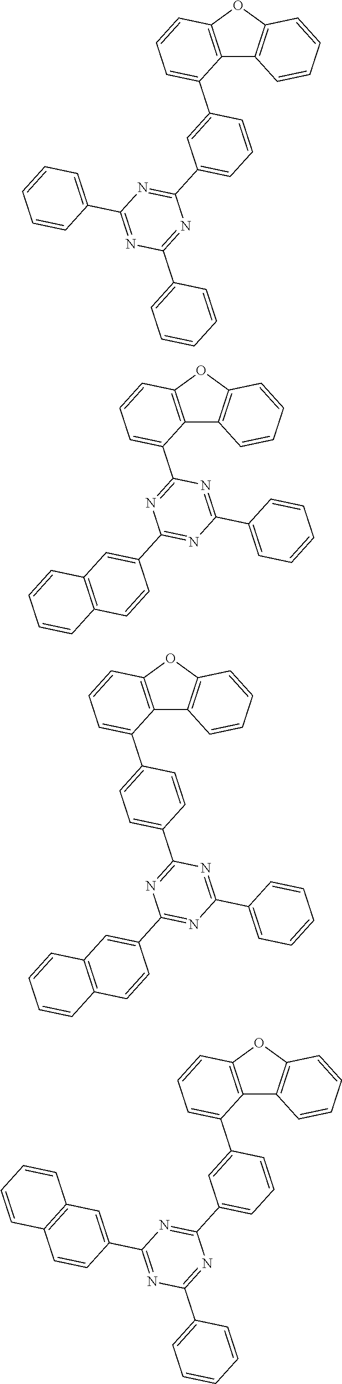

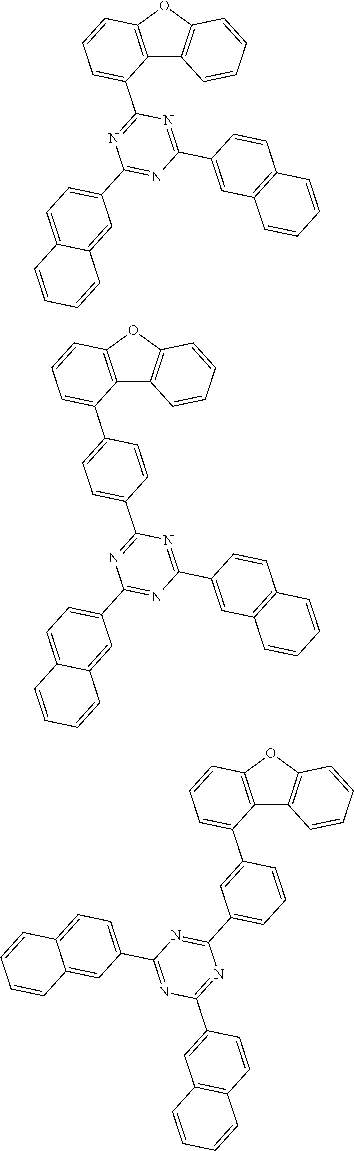

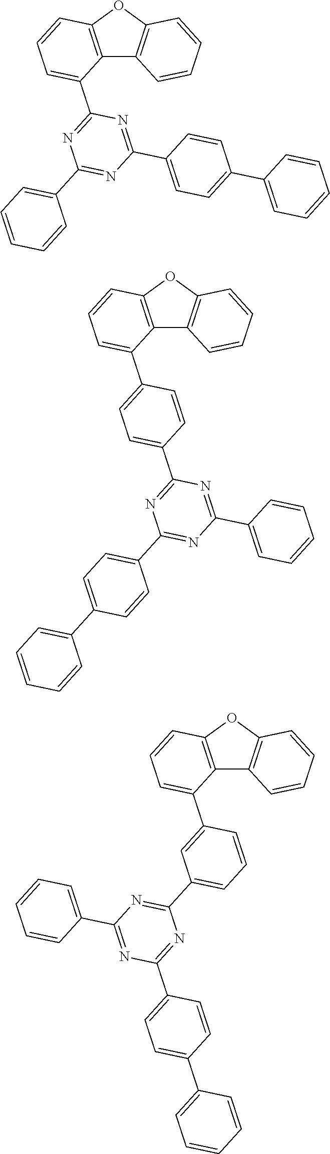 [SMILES: c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1]

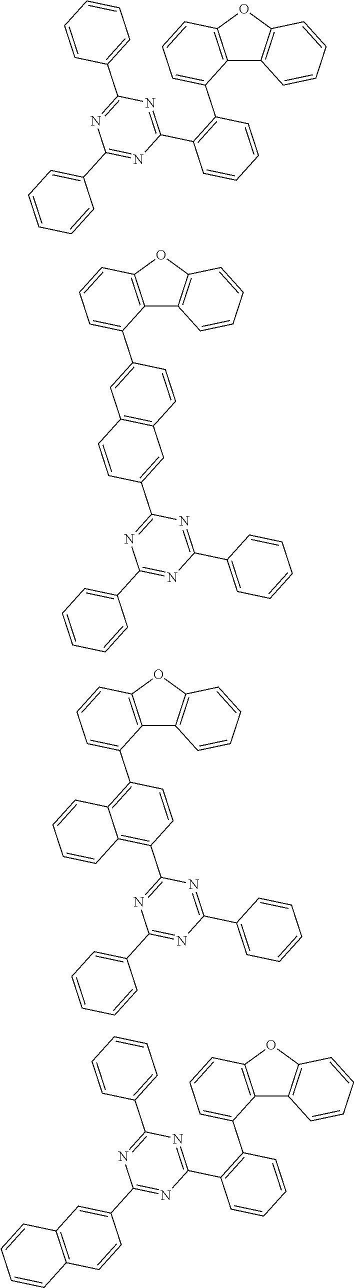

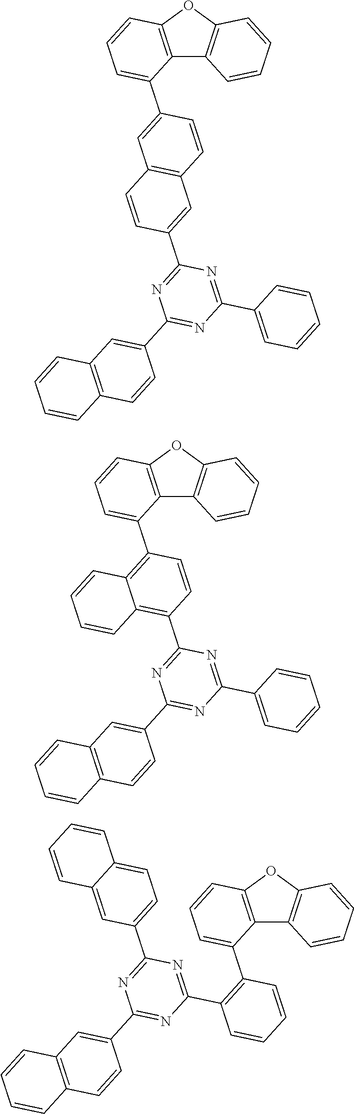

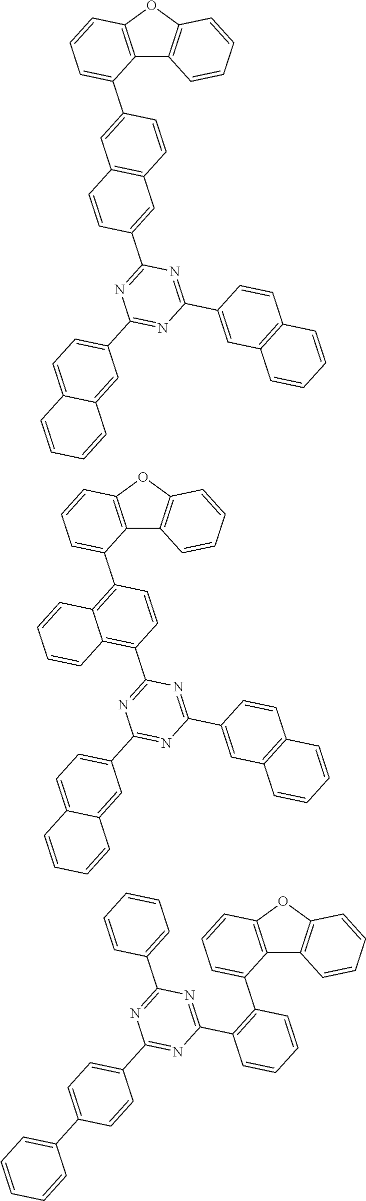

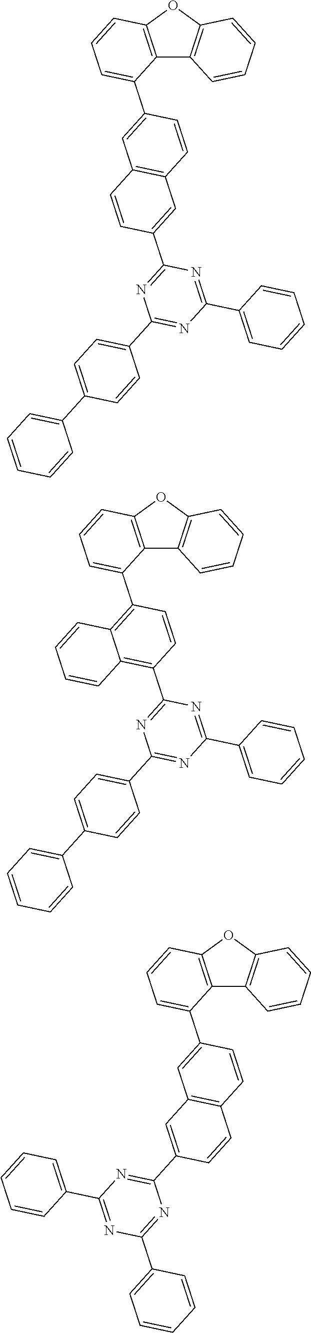 [SMILES: c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2nc(-c3ccccc3)nc(-c3ccc4ccc(-c5cccc6oc7ccccc7c56)cc4c3)n2)cc1]

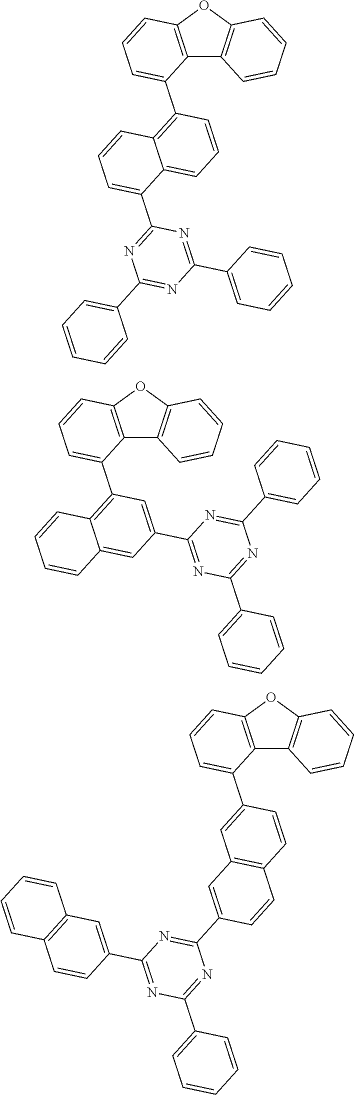

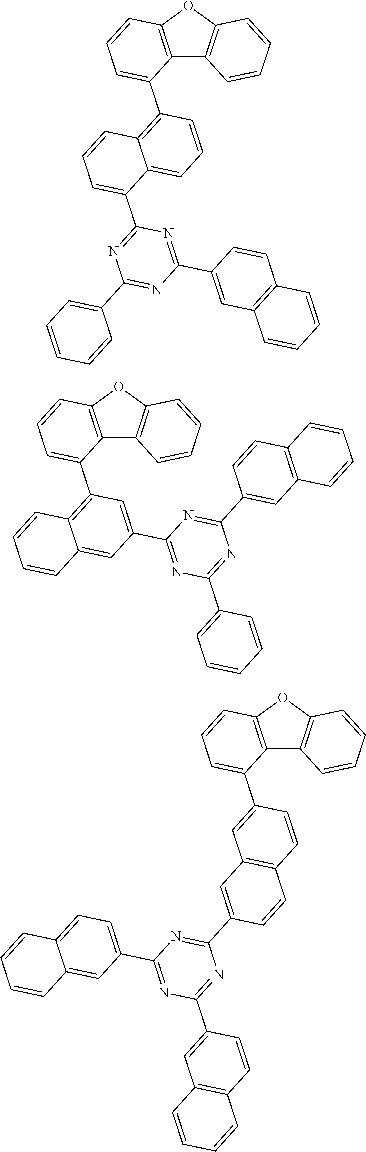

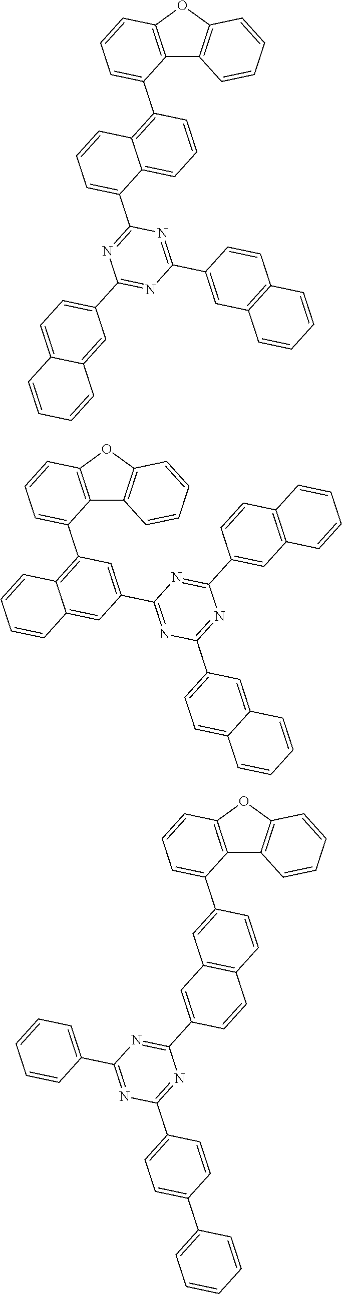

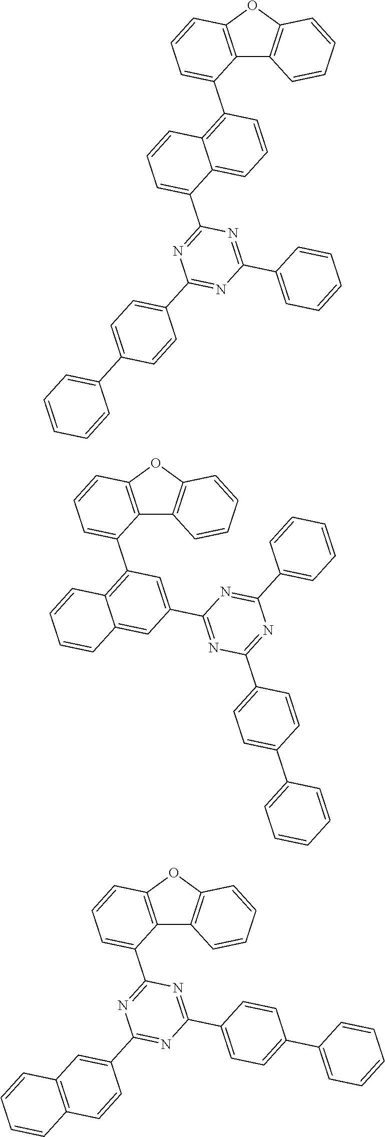

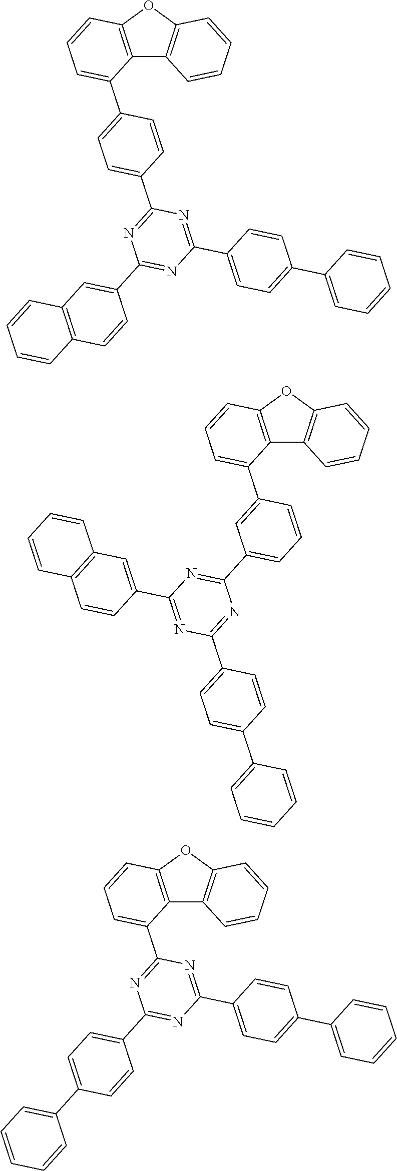

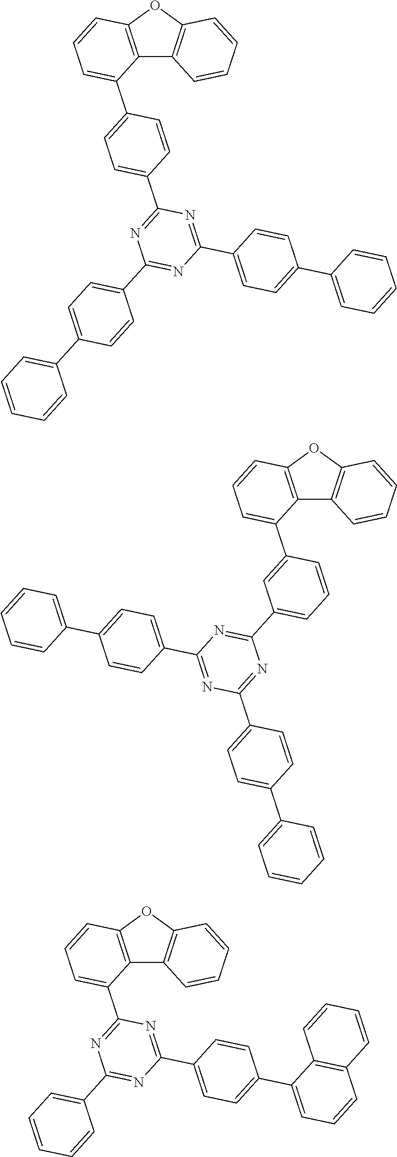 [SMILES: c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2nc(-c3ccc(-c4cccc5ccccc45)cc3)nc(-c3cccc4oc5ccccc5c34)n2)cc1]

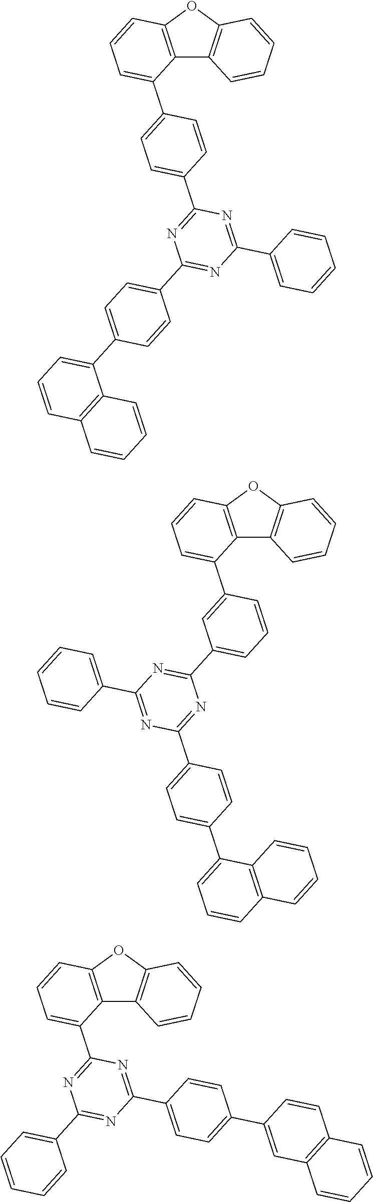 [SMILES: c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1 ; c1ccc(-c2nc(-c3ccc(-c4ccc5ccccc5c4)cc3)nc(-c3cccc4oc5ccccc5c34)n2)cc1]

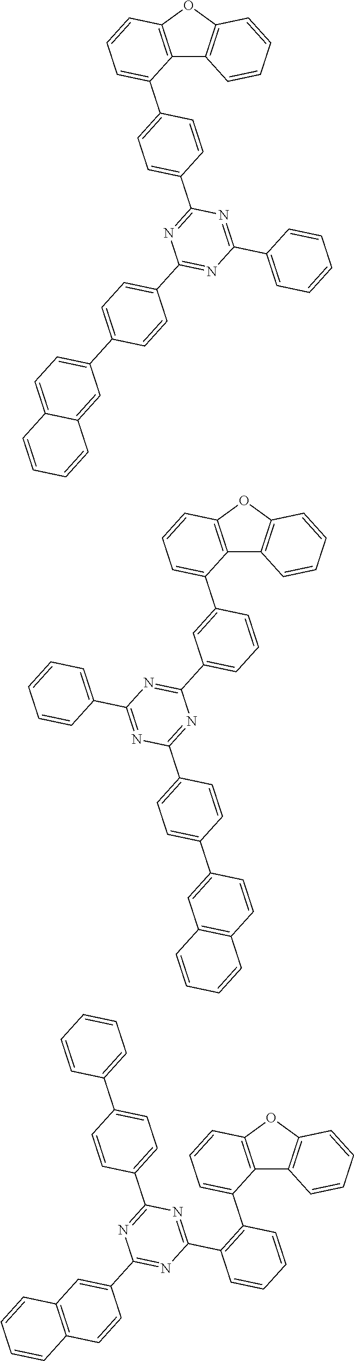 [SMILES: c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3cccc(-c4cccc5oc6ccccc6c45)c3)n2)cc1 ; c1ccc(-c2ccc(-c3nc(-c4ccc5ccccc5c4)nc(-c4ccccc4-c4cccc5oc6ccccc6c45)n3)cc2)cc1]

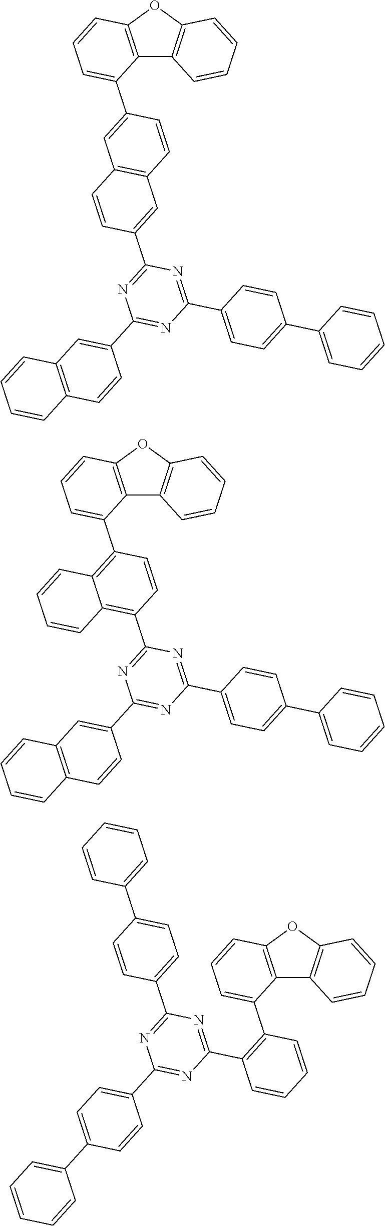

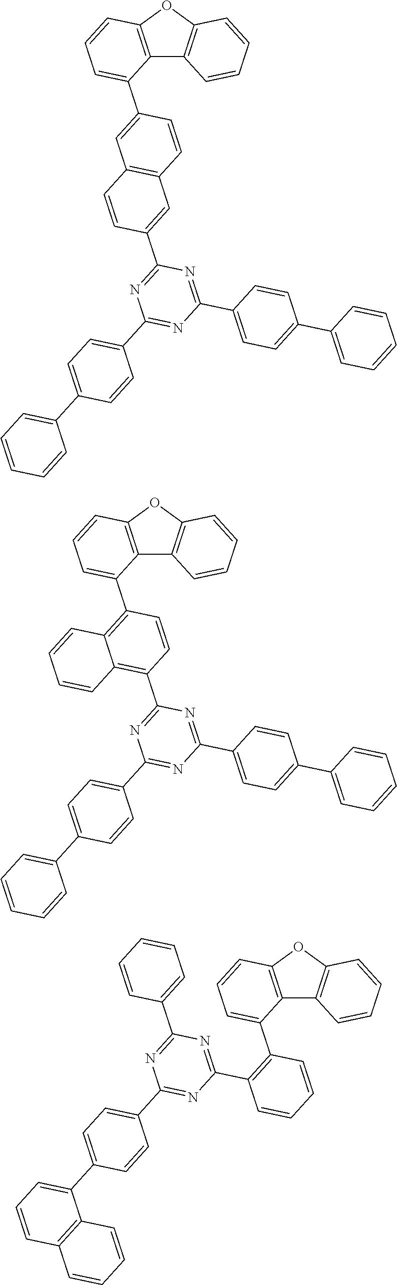 [SMILES: c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2nc(-c3ccc(-c4cccc5ccccc45)cc3)nc(-c3ccccc3-c3cccc4oc5ccccc5c34)n2)cc1]

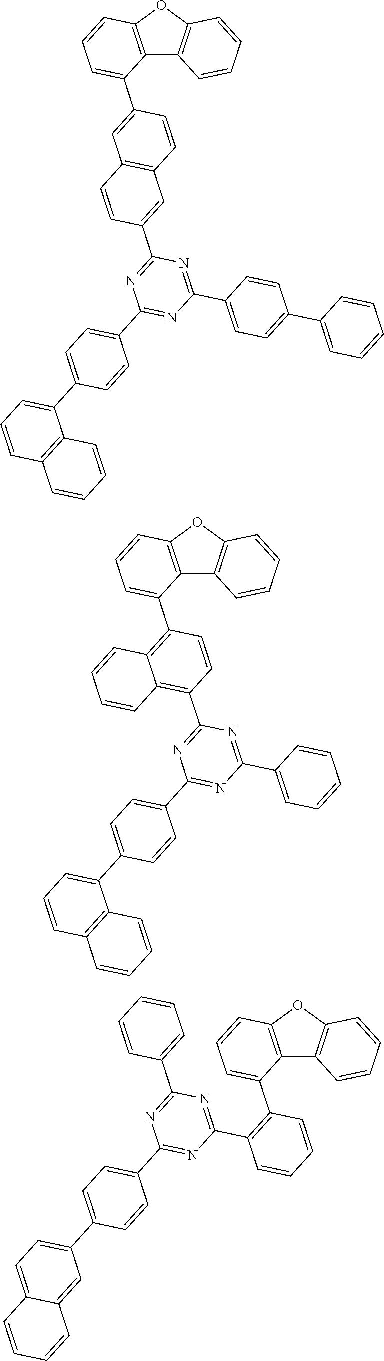

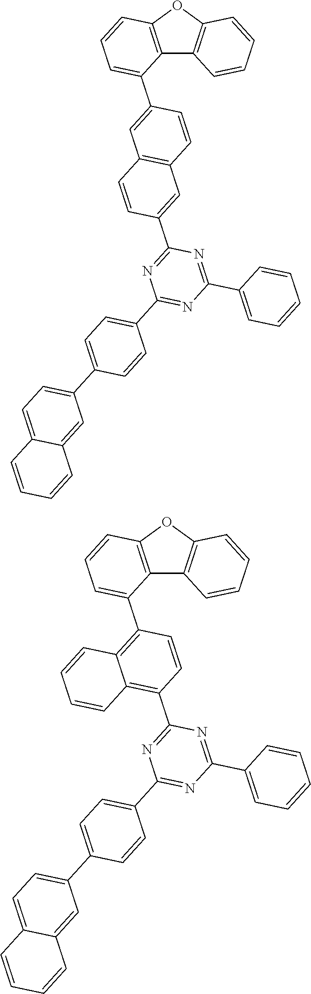 [SMILES: c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

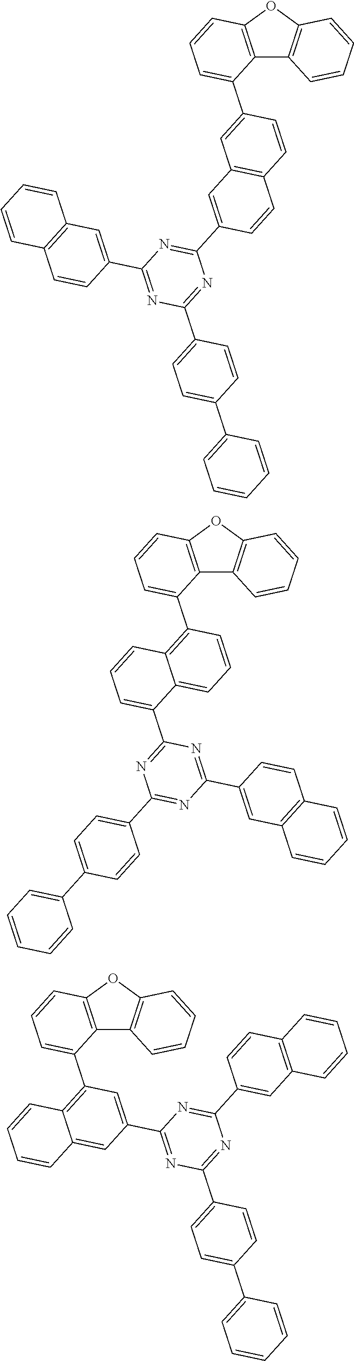

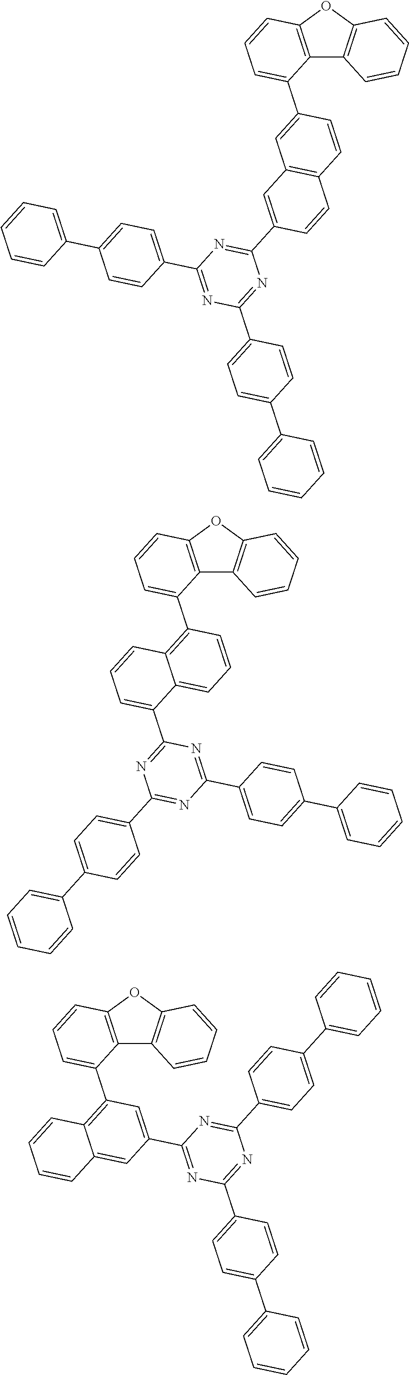

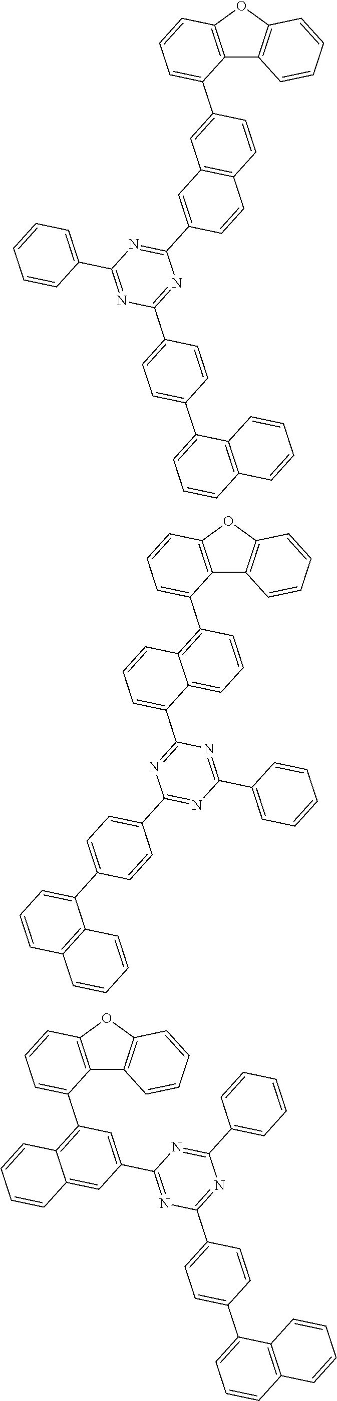

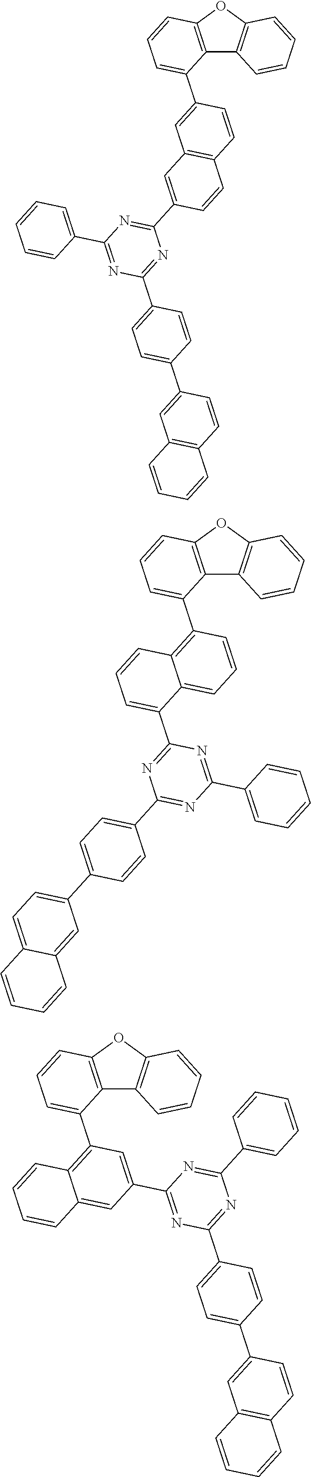

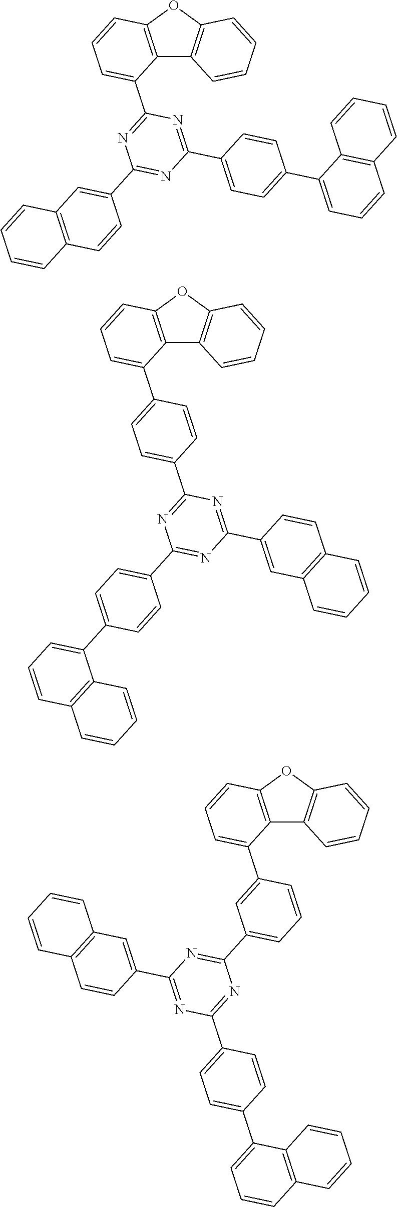 [SMILES: c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1 ; c1cc(-c2nc(-c3ccc(-c4cccc5ccccc45)cc3)nc(-c3ccc4ccccc4c3)n2)cc(-c2cccc3oc4ccccc4c23)c1]

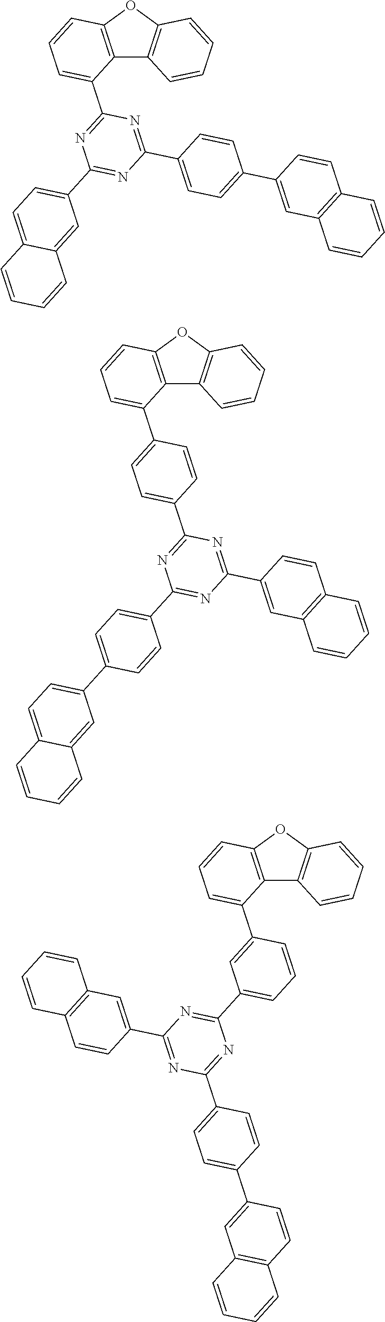 [SMILES: c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1]

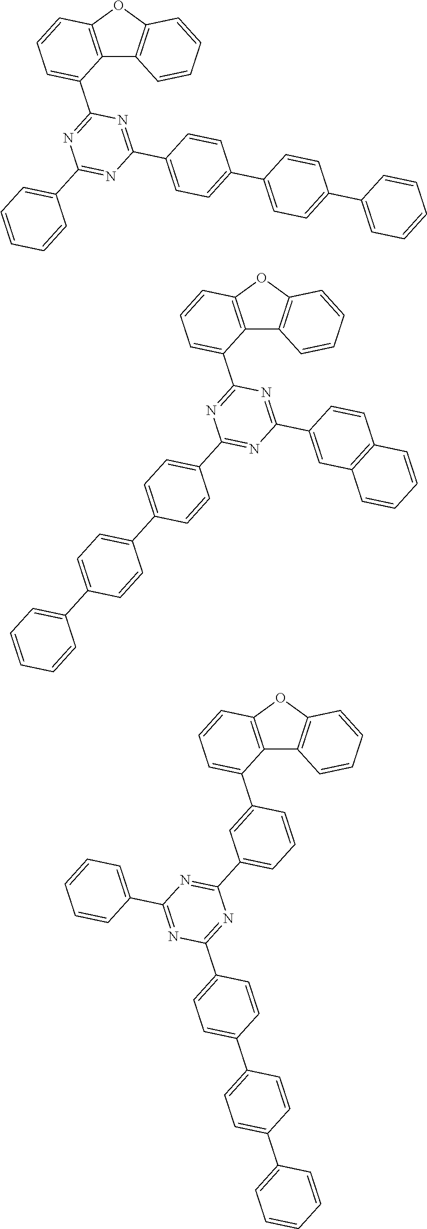 [SMILES: c1ccc(-c2ccc(-c3ccc(-c4nc(-c5ccccc5)nc(-c5cccc6oc7ccccc7c56)n4)cc3)cc2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccccc5)nc(-c5cccc(-c6cccc7oc8ccccc8c67)c5)n4)cc3)cc2)cc1]

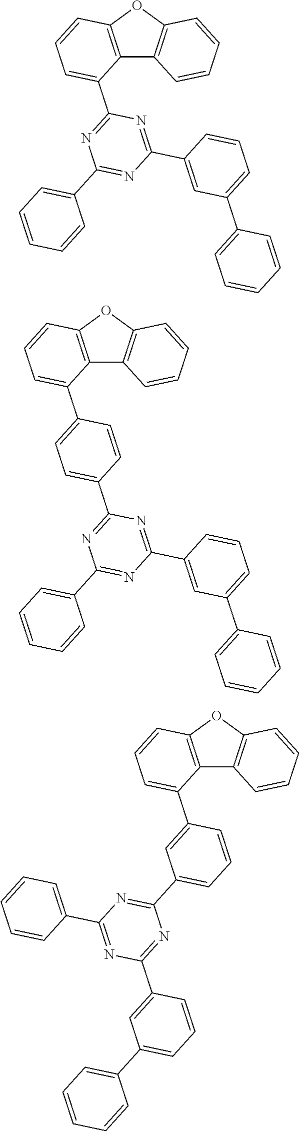

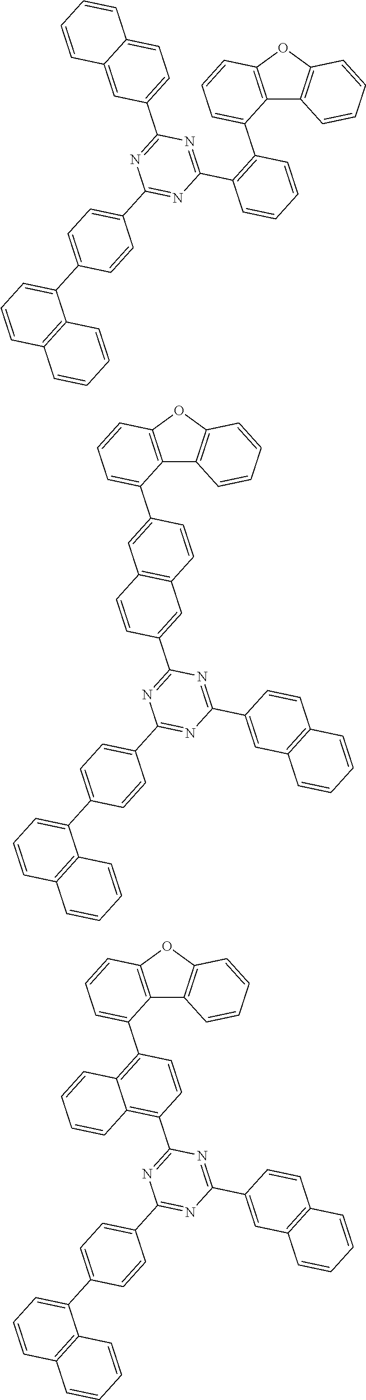

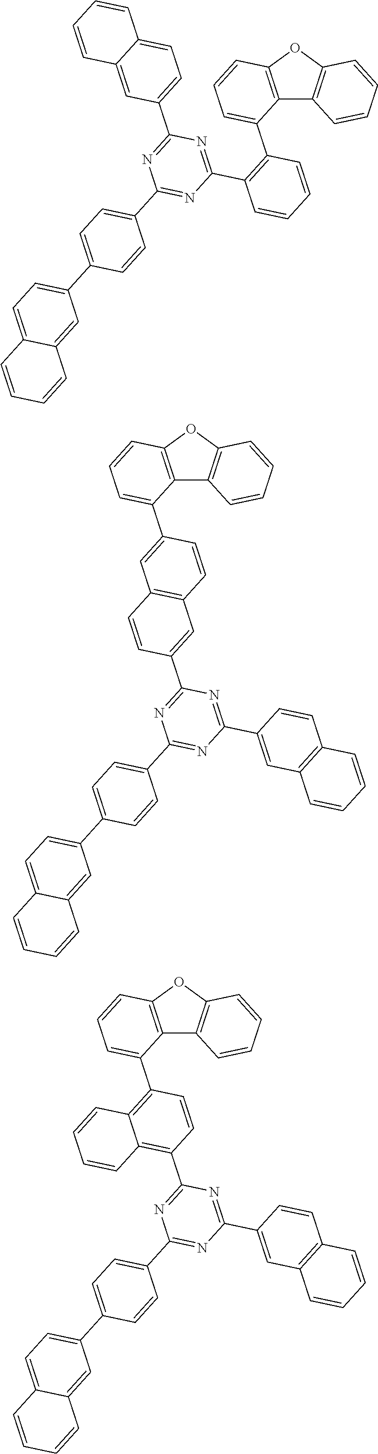 [SMILES: c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1 ; c1ccc2cc(-c3ccc(-c4nc(-c5ccc6ccccc6c5)nc(-c5ccc(-c6cccc7oc8ccccc8c67)c6ccccc56)n4)cc3)ccc2c1]

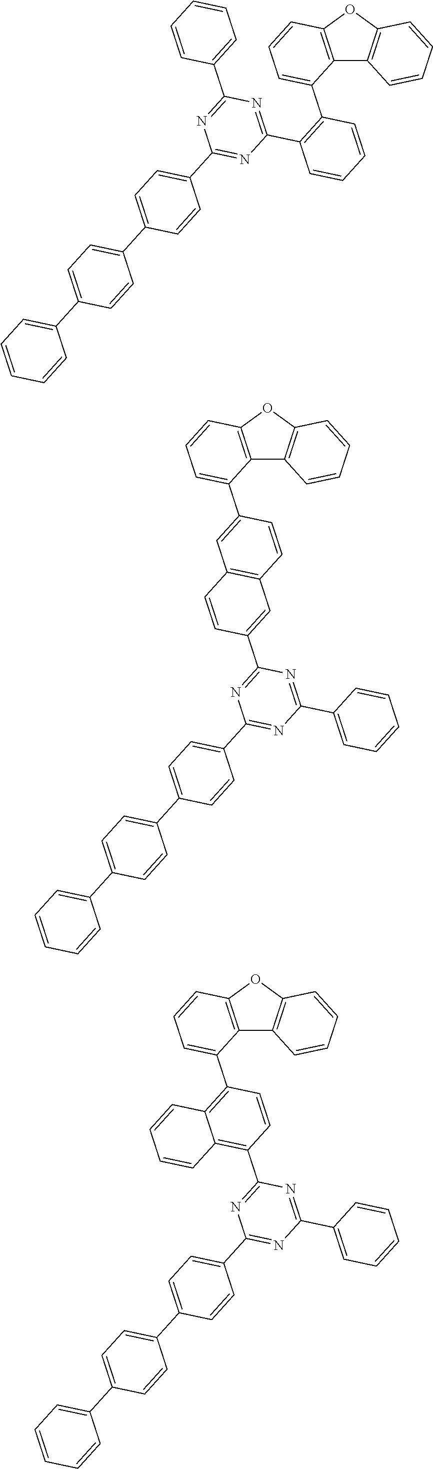 [SMILES: c1ccc(-c2ccc(-c3ccc(-c4nc(-c5ccccc5)nc(-c5ccccc5-c5cccc6oc7ccccc7c56)n4)cc3)cc2)cc1 ; c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

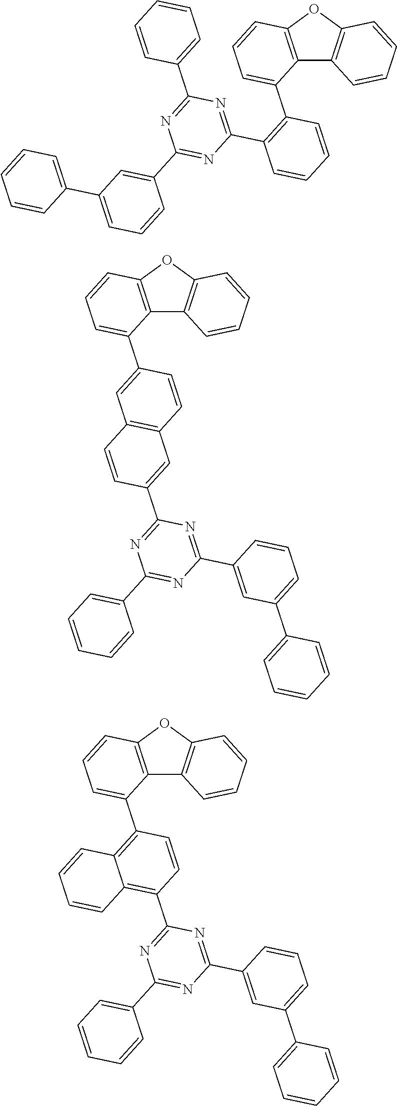

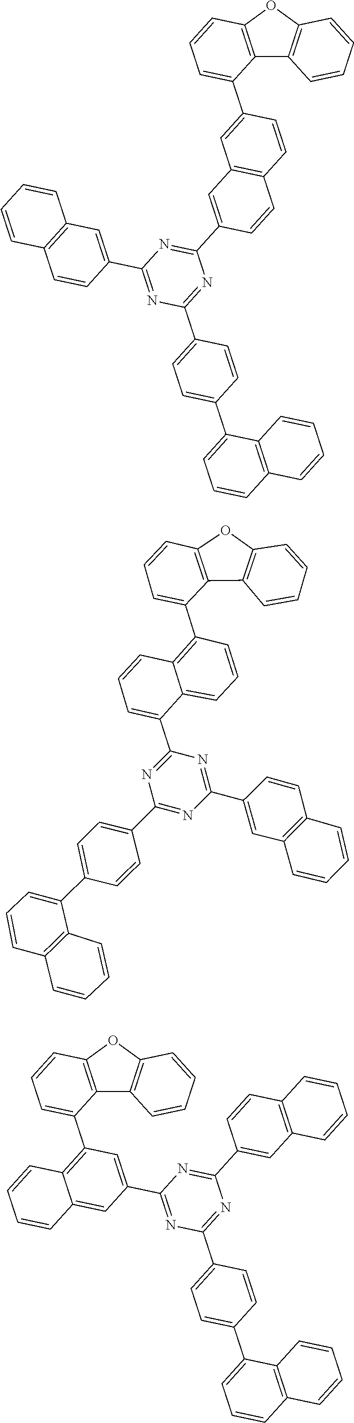

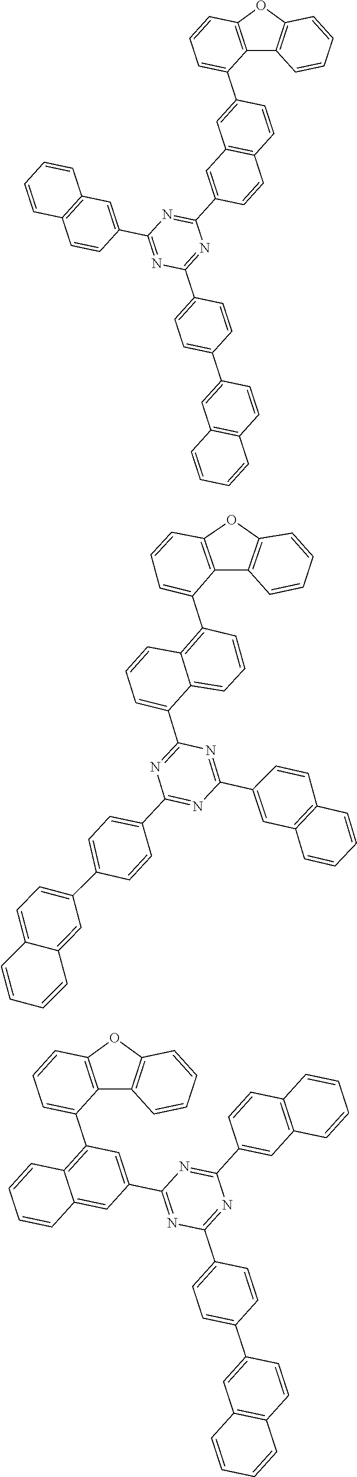

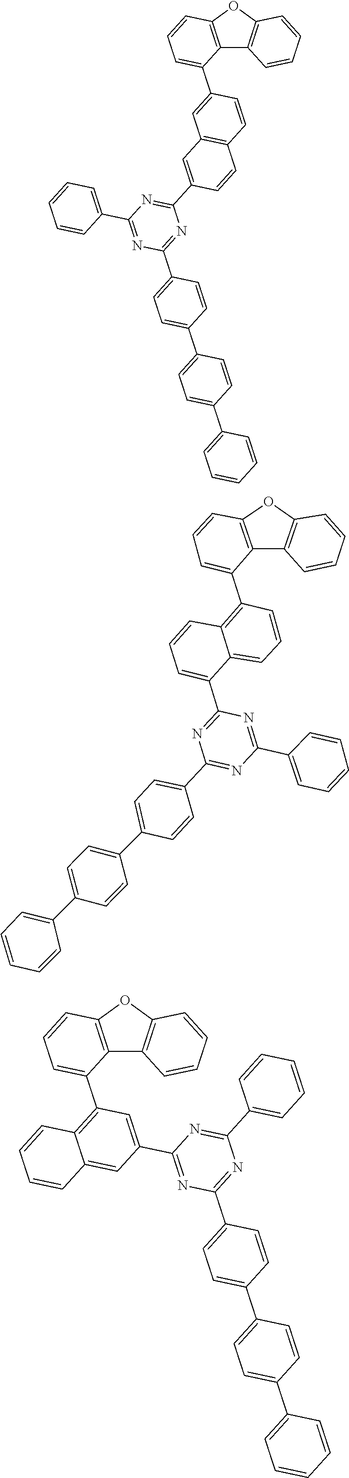

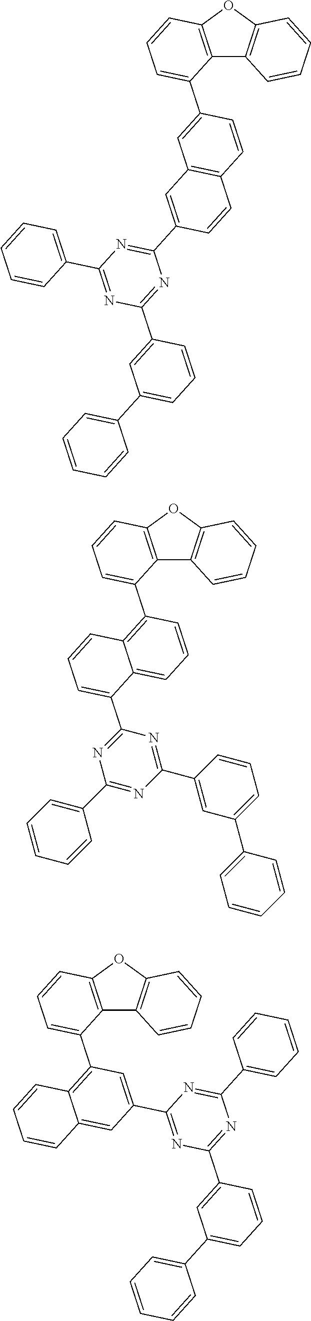

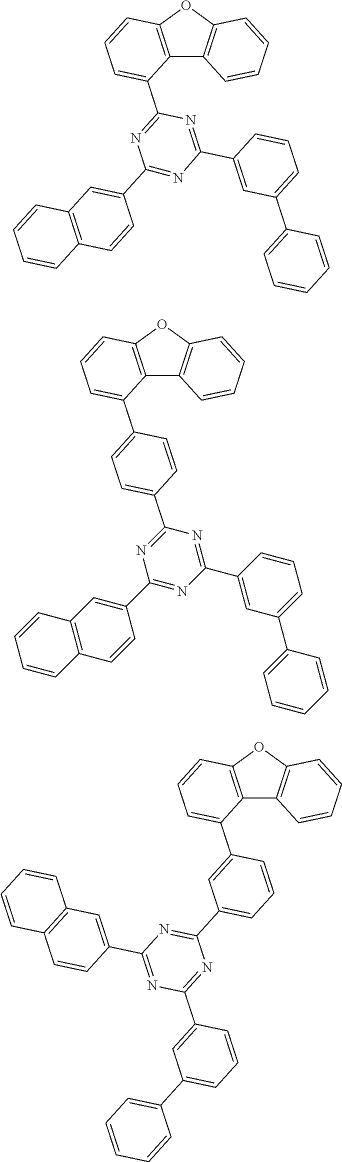

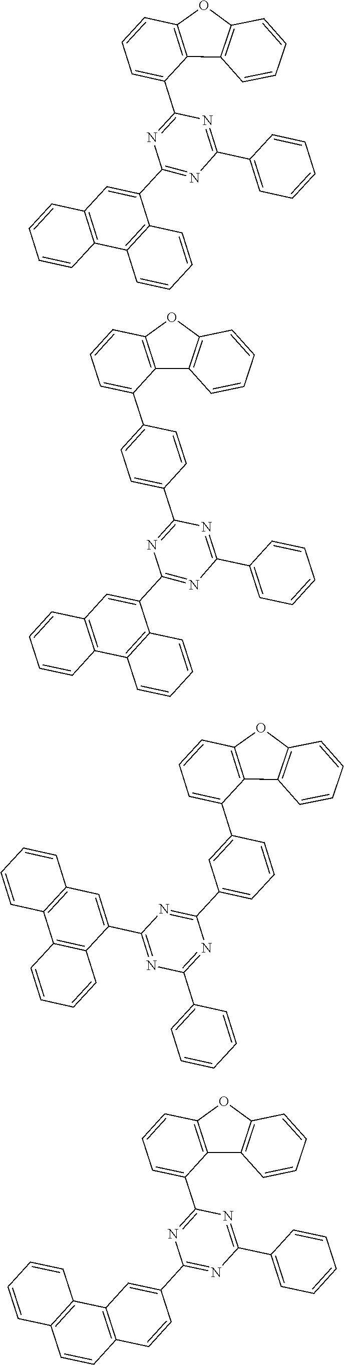

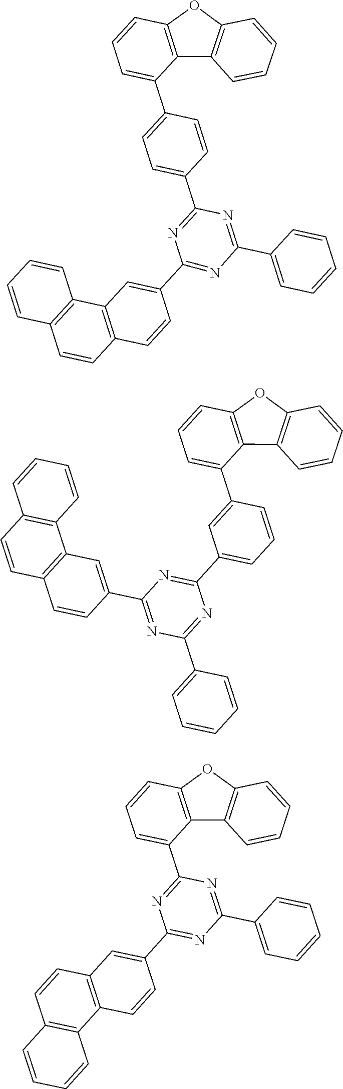

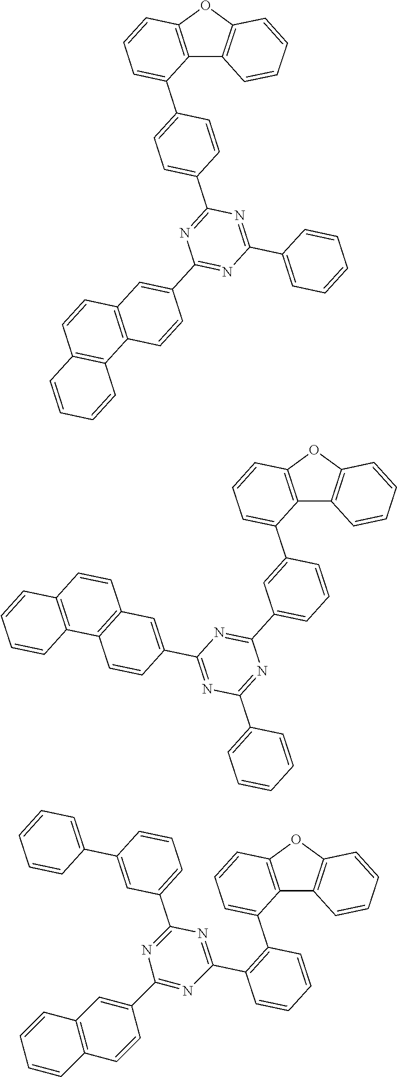 [SMILES: c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1 ; c1ccc(-c2cccc(-c3nc(-c4ccc5ccccc5c4)nc(-c4ccccc4-c4cccc5oc6ccccc6c45)n3)c2)cc1]

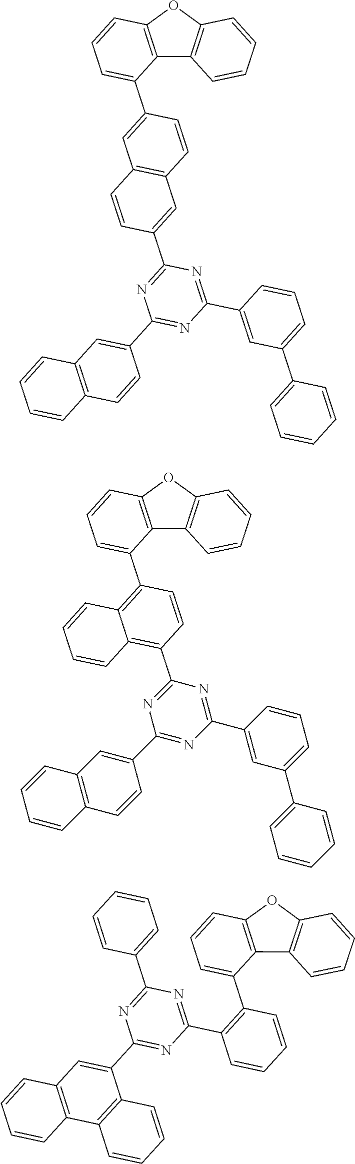

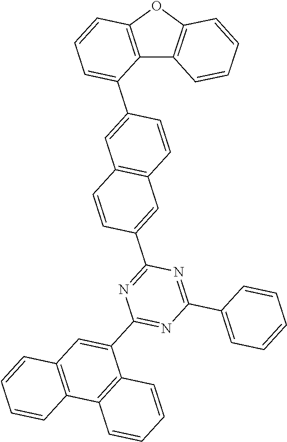 [SMILES: c1ccc(-c2nc(-c3ccc4cc(-c5cccc6oc7ccccc7c56)ccc4c3)nc(-c3cc4ccccc4c4ccccc34)n2)cc1]

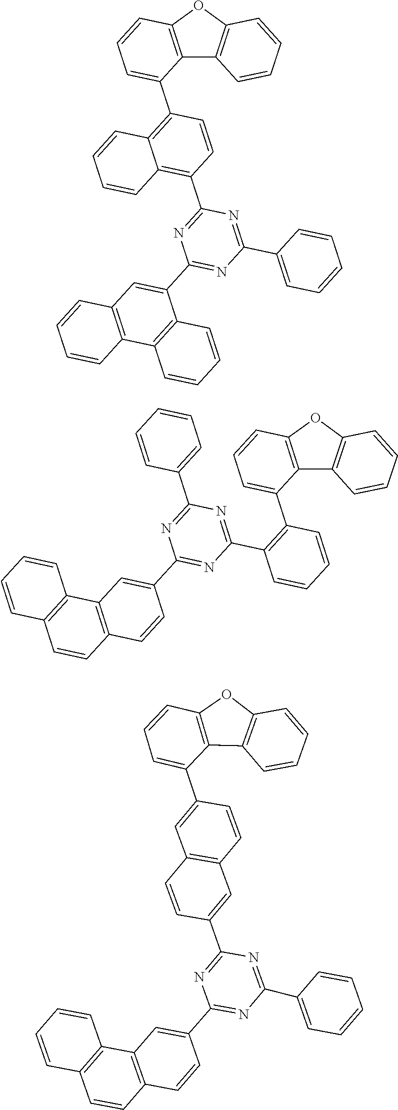

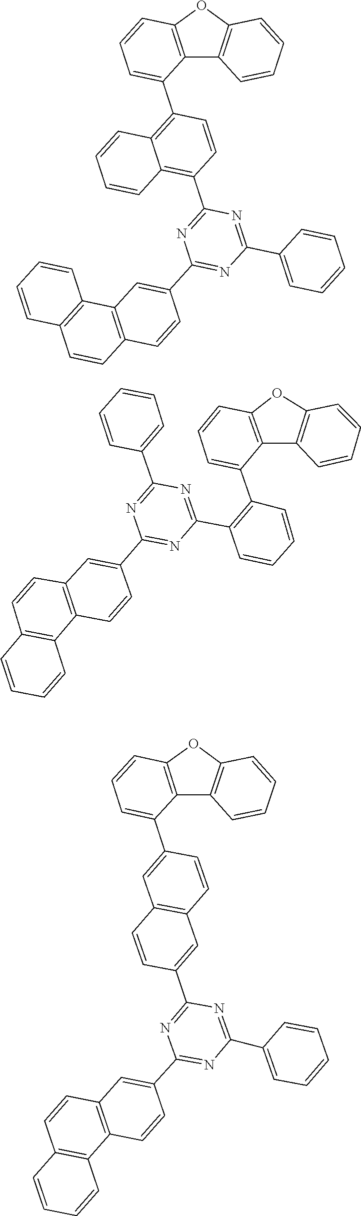

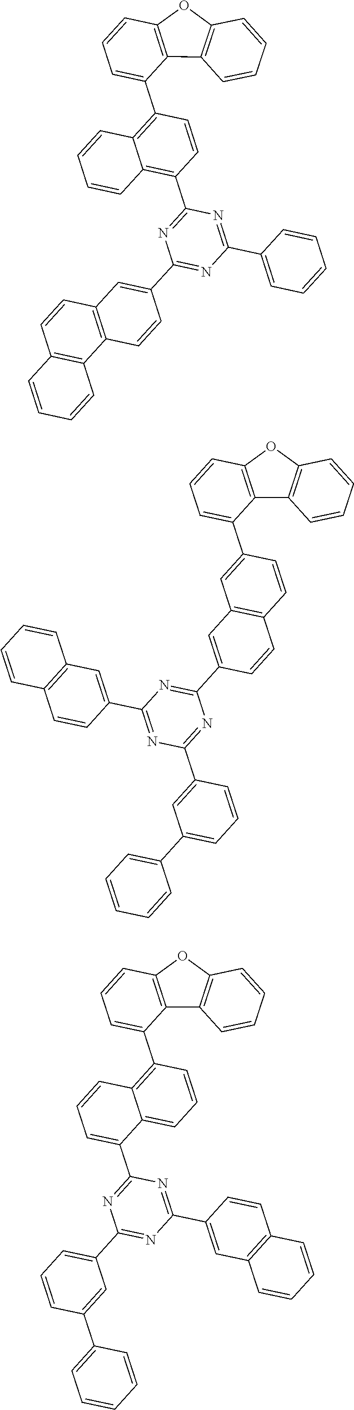 [SMILES: c1ccc(-c2nc(-c3ccc4c(ccc5ccccc54)c3)nc(-c3ccc(-c4cccc5oc6ccccc6c45)c4ccccc34)n2)cc1 ; c1ccc(-c2cccc(-c3nc(-c4ccc5ccccc5c4)nc(-c4ccc5ccc(-c6cccc7oc8ccccc8c67)cc5c4)n3)c2)cc1 ; c1ccc(-c2cccc(-c3nc(-c4ccc5ccccc5c4)nc(-c4cccc5c(-c6cccc7oc8ccccc8c67)cccc45)n3)c2)cc1]

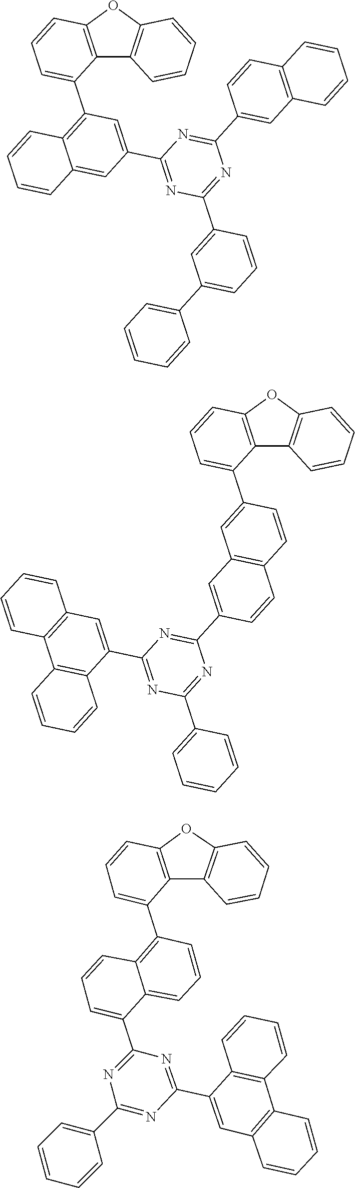

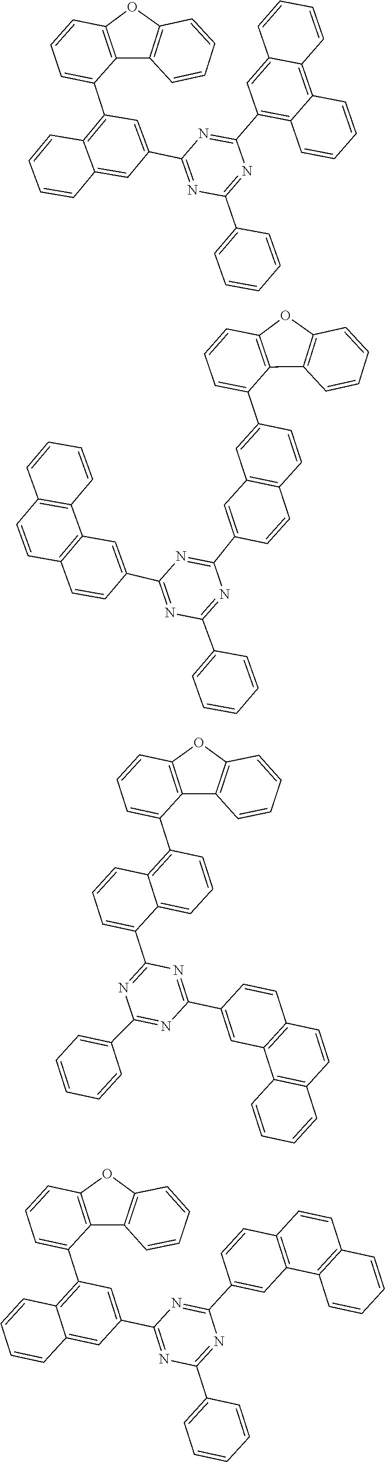

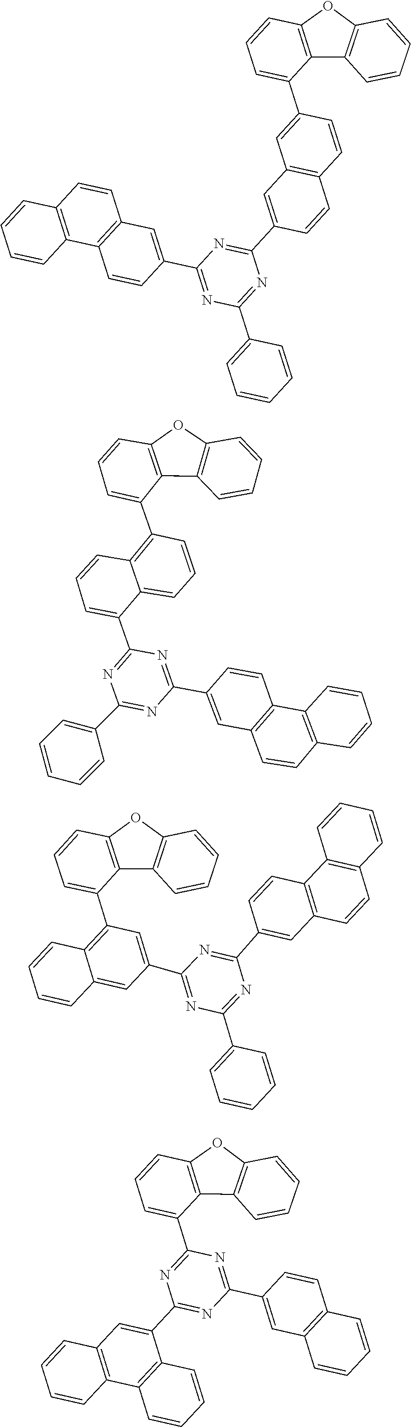

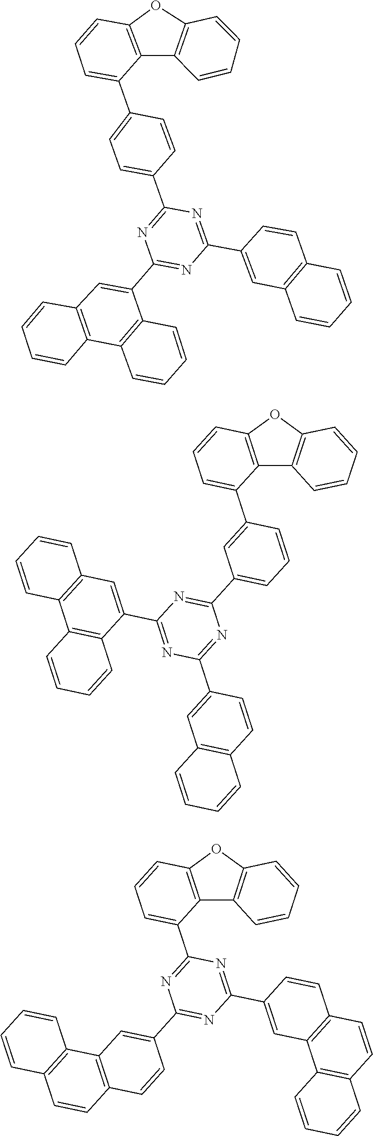

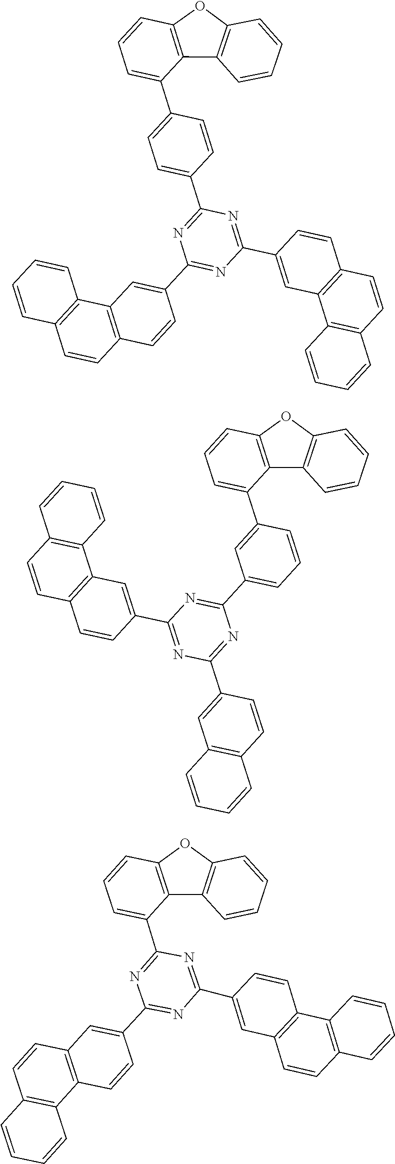

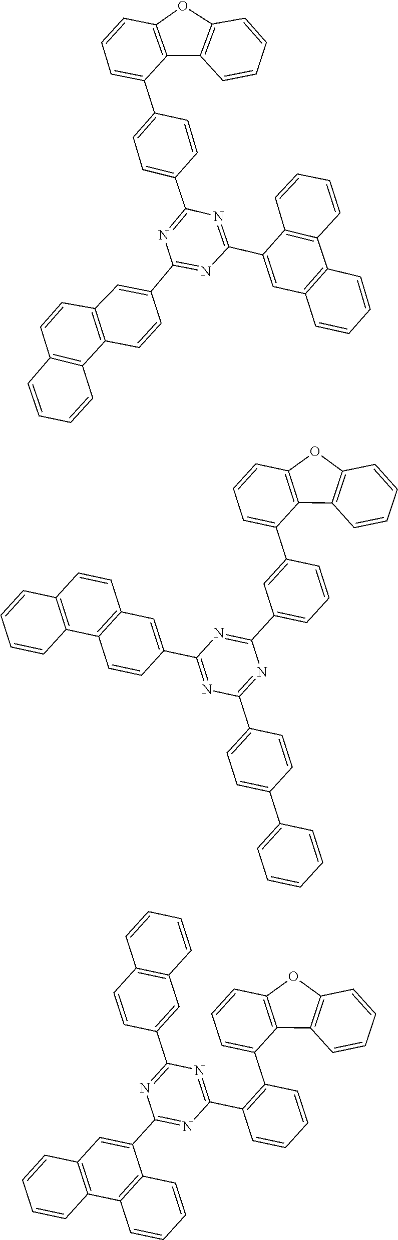 [SMILES: c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc4ccccc4c3)nc(-c3cc4ccccc4c4ccccc34)n2)c1]

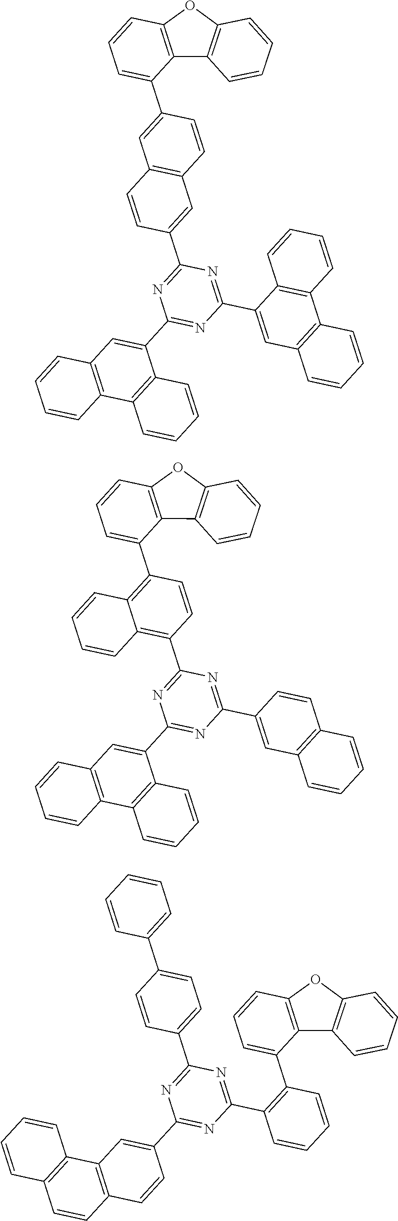

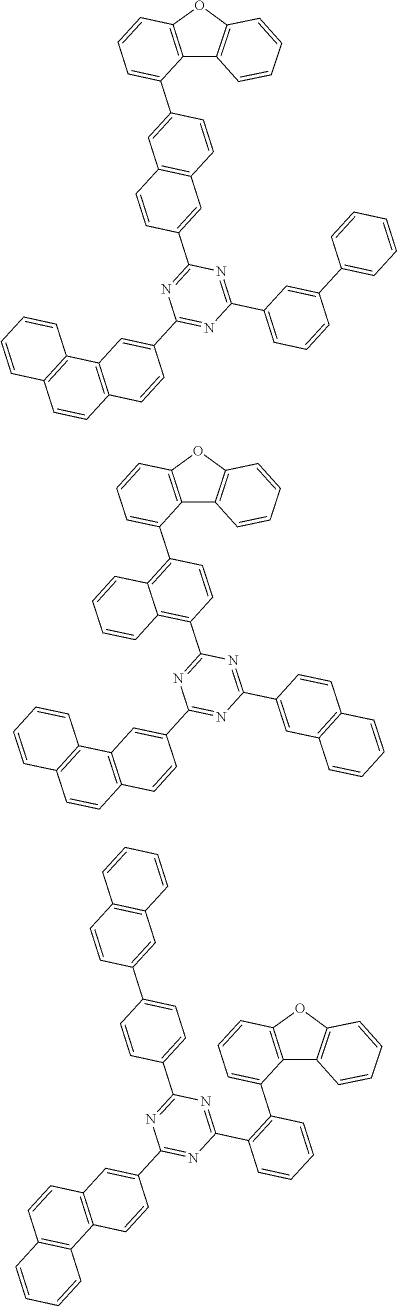

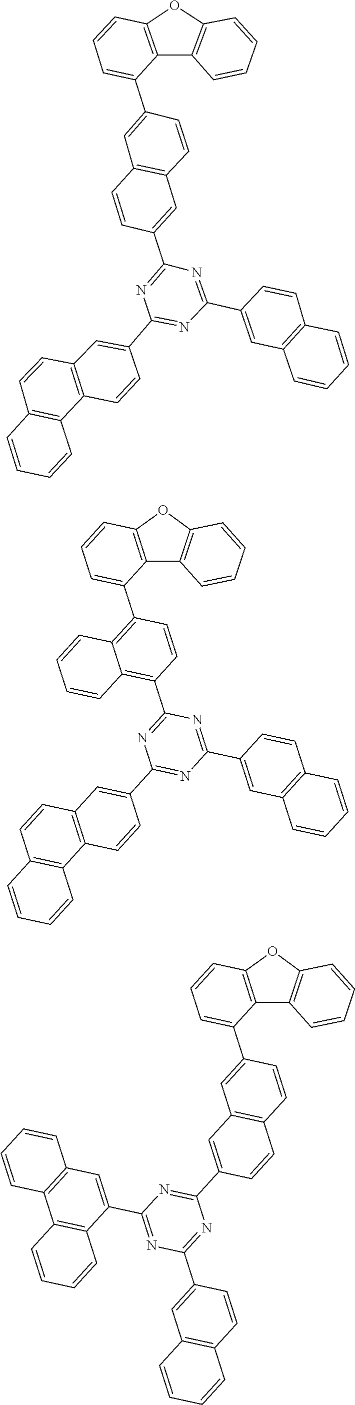 [SMILES: c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4cc5ccccc5c5ccccc45)n3)ccc2c1]

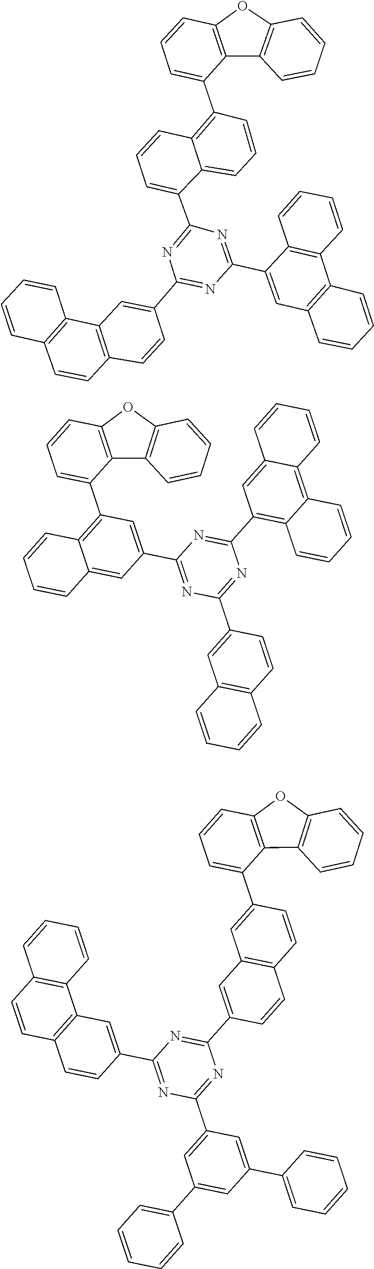

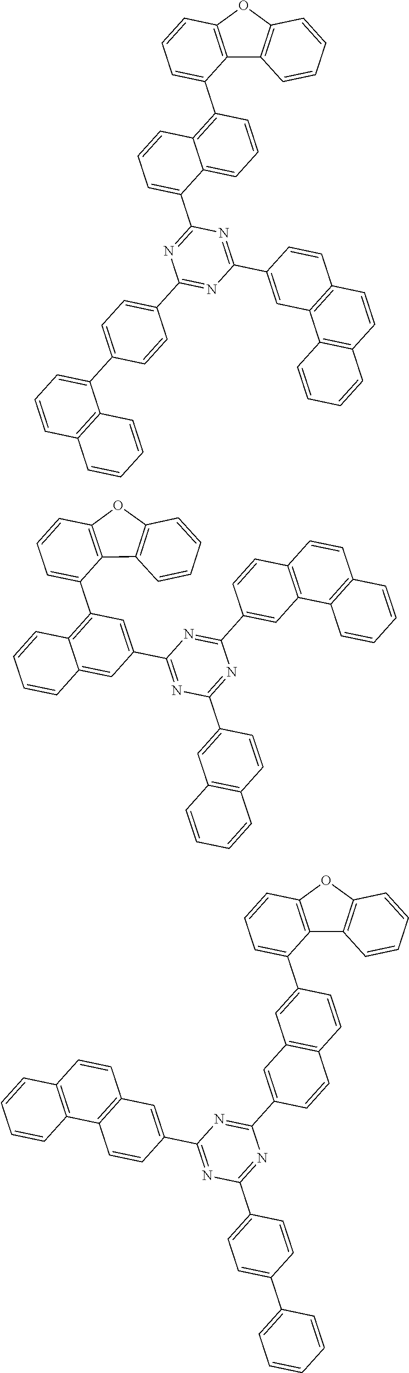

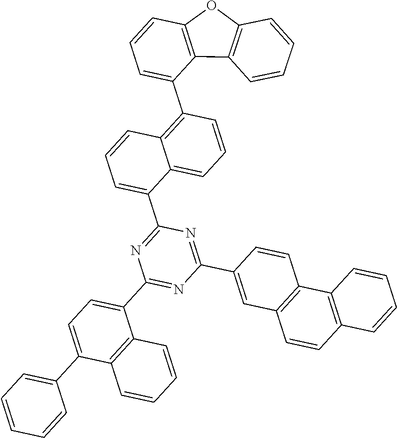 [SMILES: c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1]

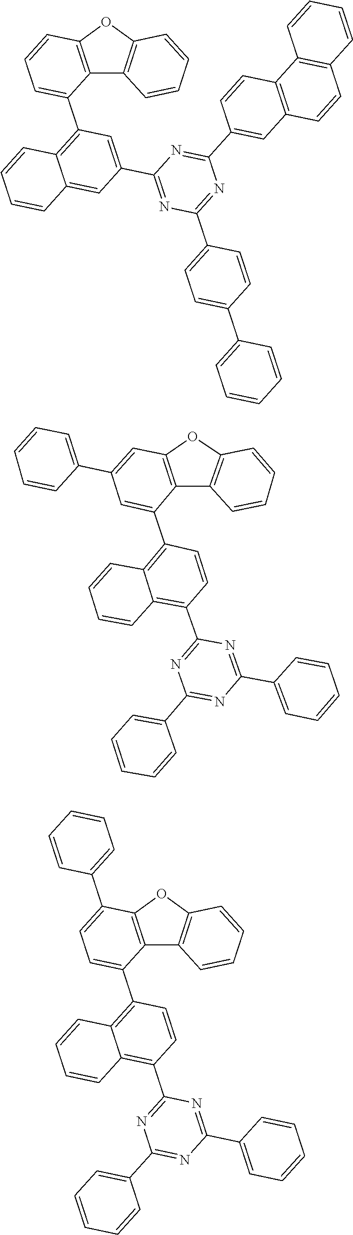

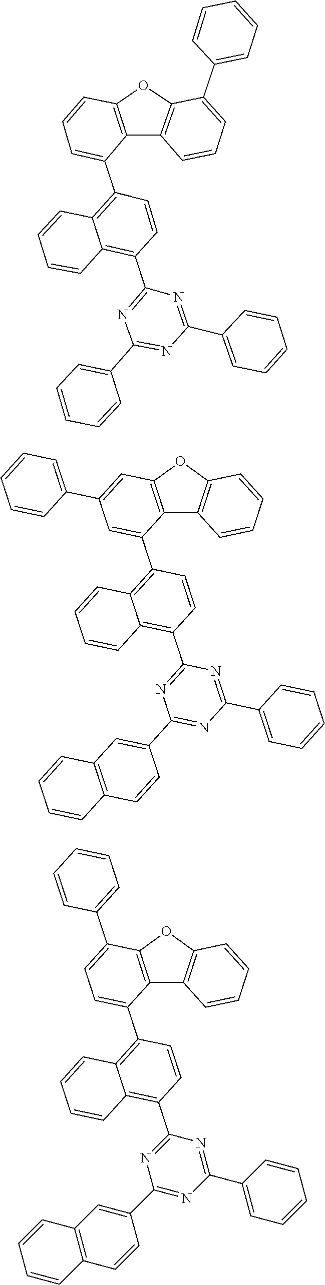

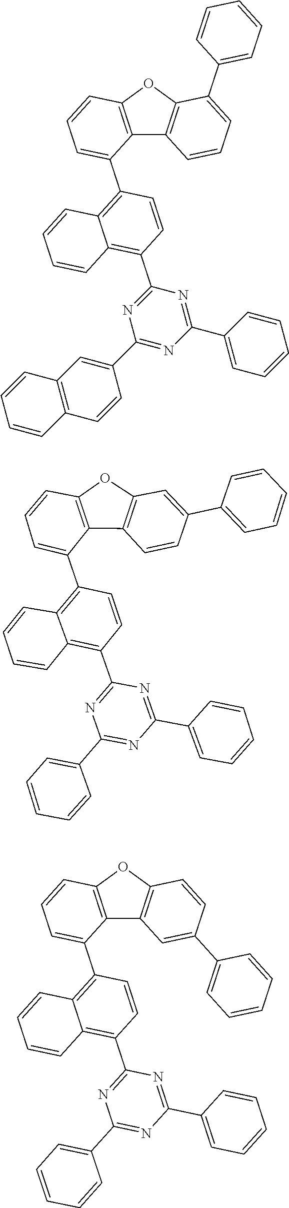 [SMILES: c1ccc(-c2nc(-c3ccc4ccccc4c3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3oc4cccc(-c5ccc(-c6nc(-c7ccccc7)nc(-c7ccccc7)n6)c6ccccc56)c4c3c2)cc1]

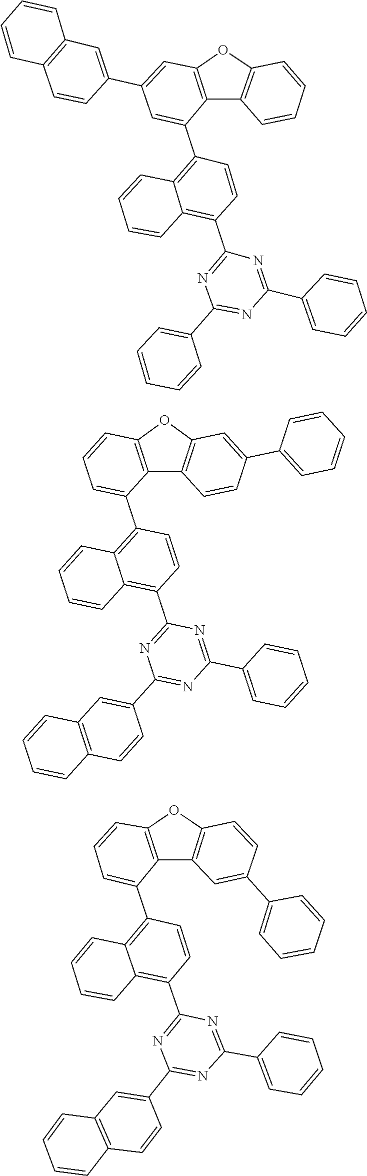

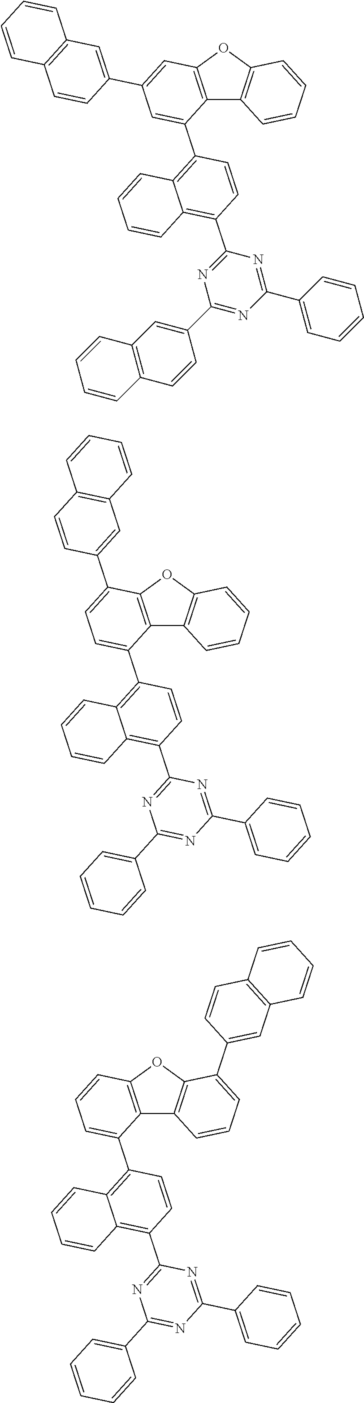

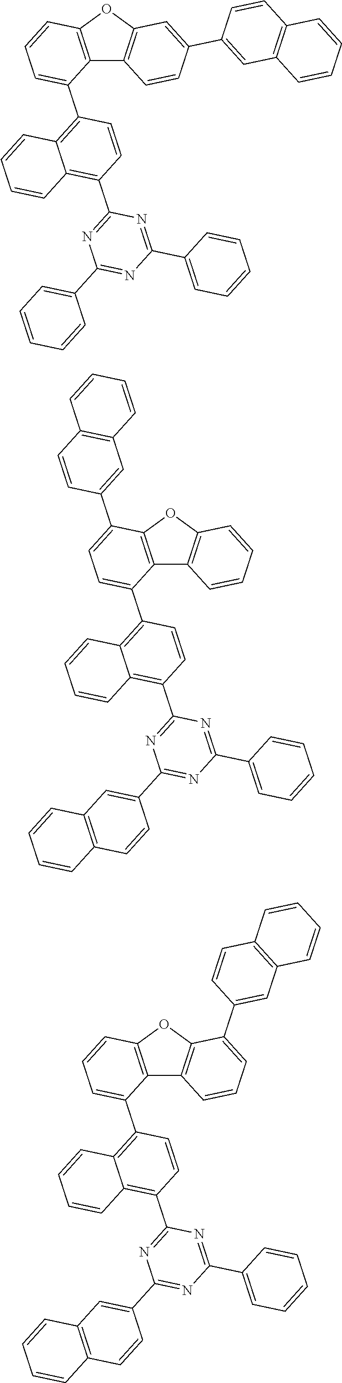

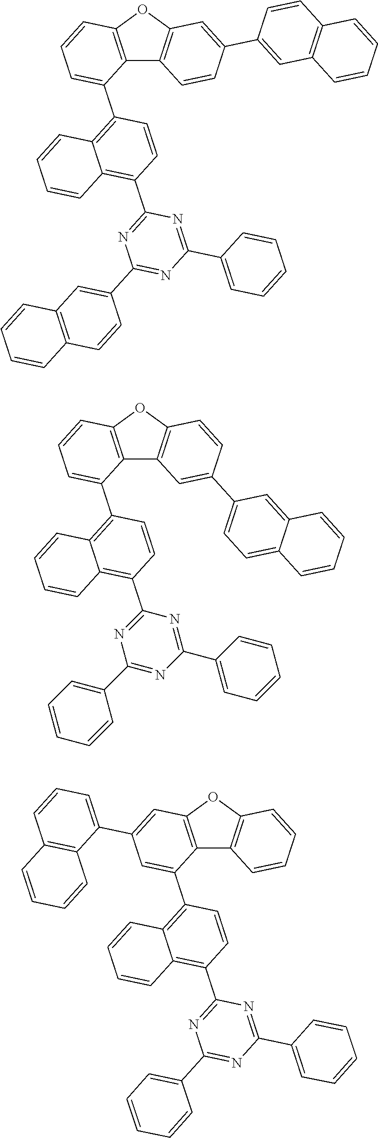 [SMILES: c1ccc(-c2nc(-c3ccc4ccccc4c3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1]

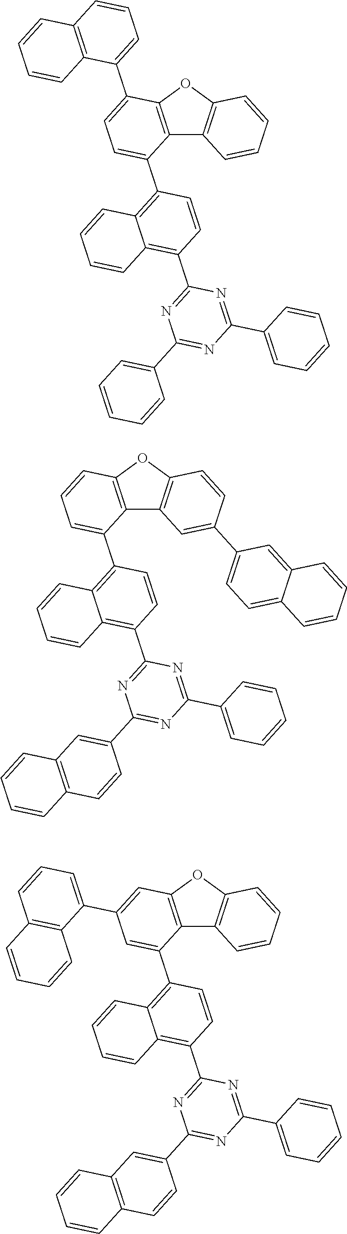

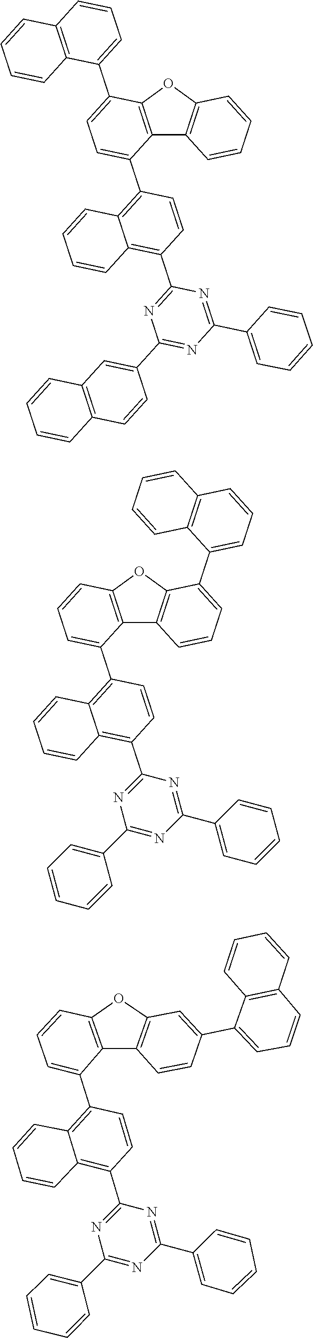 [SMILES: c1ccc(-c2nc(-c3ccc4ccccc4c3)nc(-c3ccc(-c4ccc(-c5cccc6ccccc56)c5oc6ccccc6c45)c4ccccc34)n2)cc1 ; c1ccc(-c2nc(-c3ccccc3)nc(-c3ccc(-c4cccc5oc6c(-c7cccc8ccccc78)cccc6c45)c4ccccc34)n2)cc1 ; c1ccc(-c2nc(-c3ccccc3)nc(-c3ccc(-c4cccc5oc6cc(-c7cccc8ccccc78)ccc6c45)c4ccccc34)n2)cc1]

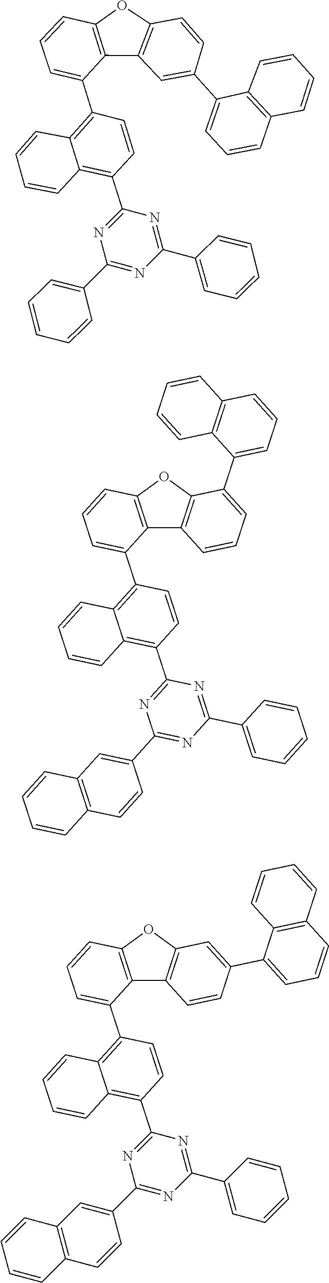 [SMILES: c1ccc(-c2nc(-c3ccccc3)nc(-c3ccc(-c4cccc5oc6ccc(-c7cccc8ccccc78)cc6c45)c4ccccc34)n2)cc1 ; c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1]

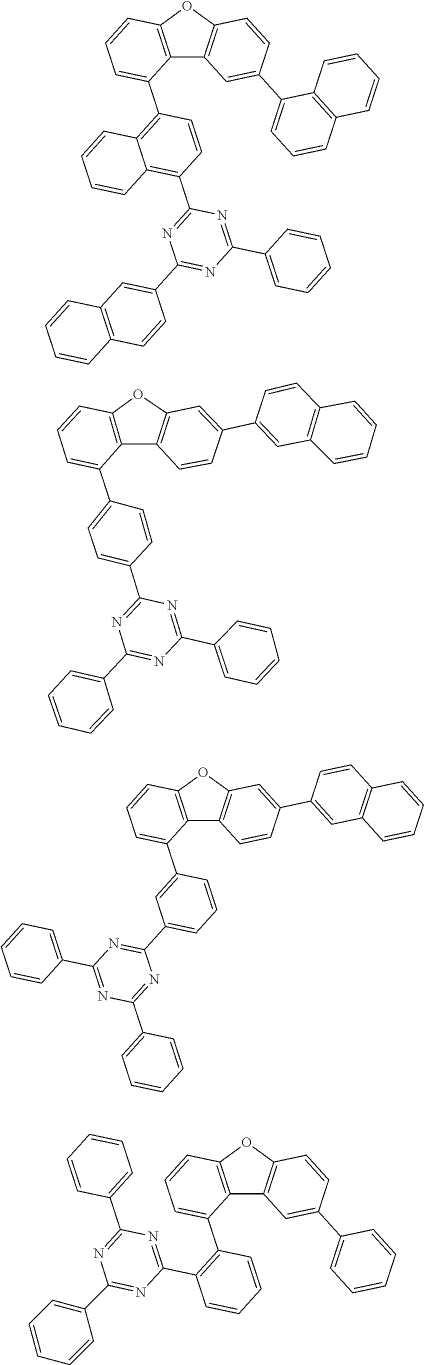

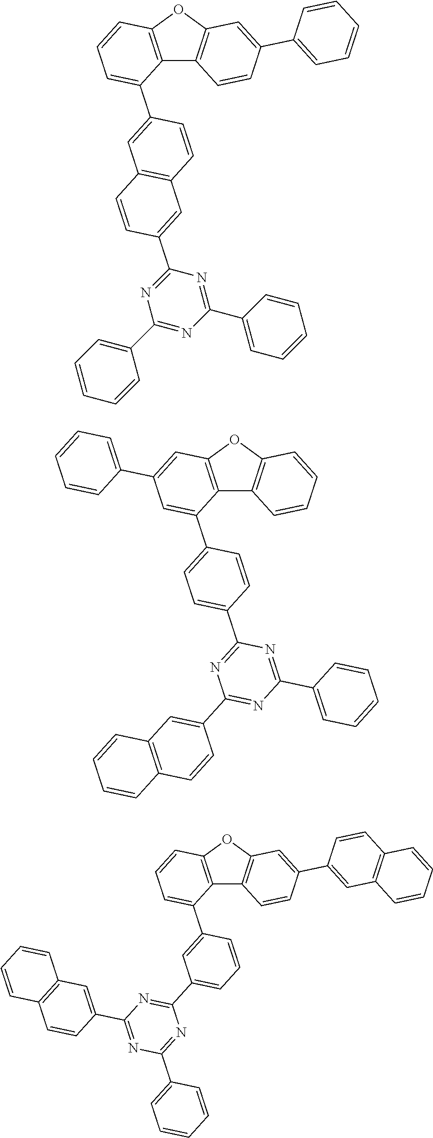

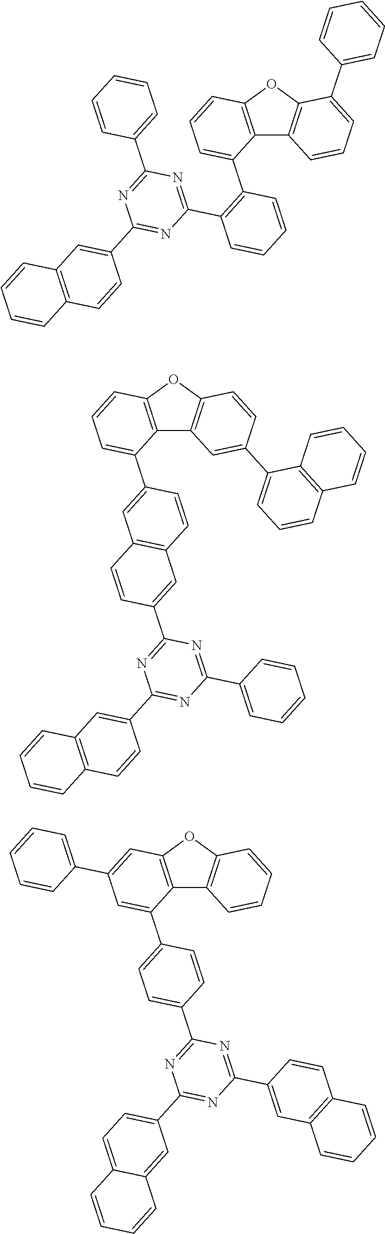

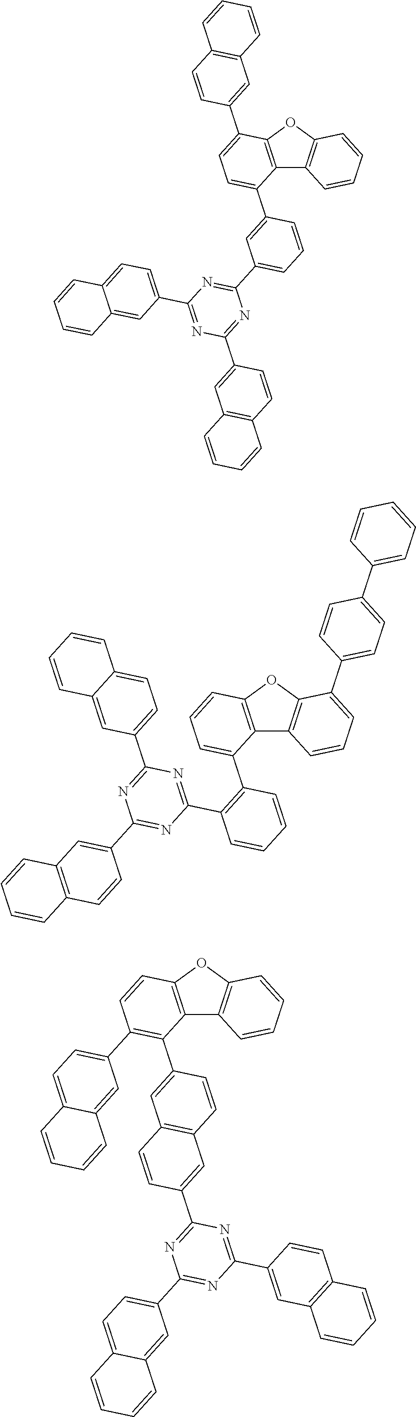 [SMILES: c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1 ; c1ccc2cc(-c3nc(-c4ccc5ccccc5c4)nc(-c4ccc5cc(-c6c(-c7ccc8ccccc8c7)ccc7oc8ccccc8c67)ccc5c4)n3)ccc2c1]

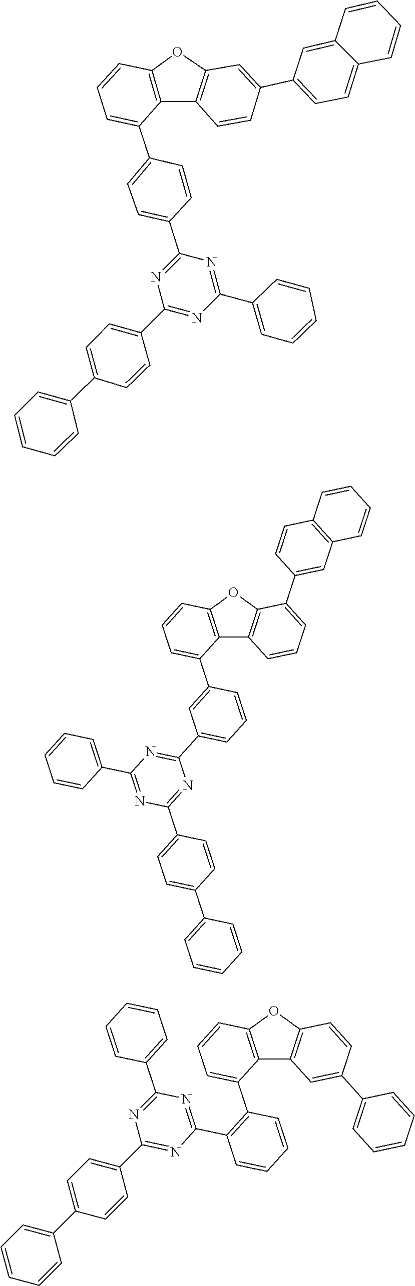 [SMILES: c1ccc(-c2ccc(-c3nc(-c4ccccc4)nc(-c4ccc(-c5cccc6oc7cc(-c8ccc9ccccc9c8)ccc7c56)cc4)n3)cc2)cc1 ; c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2ccc(-c3nc(-c4ccccc4)nc(-c4ccccc4-c4cccc5oc6ccc(-c7ccccc7)cc6c45)n3)cc2)cc1]

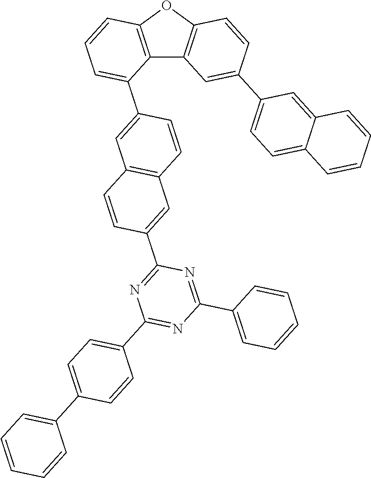 [SMILES: c1ccc(-c2ccc(-c3nc(-c4ccccc4)nc(-c4ccc5cc(-c6cccc7oc8ccc(-c9ccc%10ccccc%10c9)cc8c67)ccc5c4)n3)cc2)cc1]

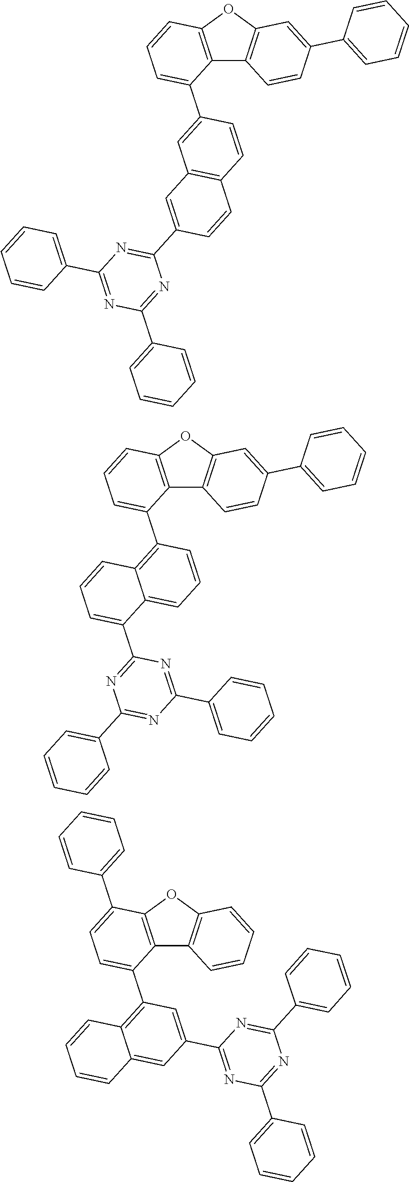

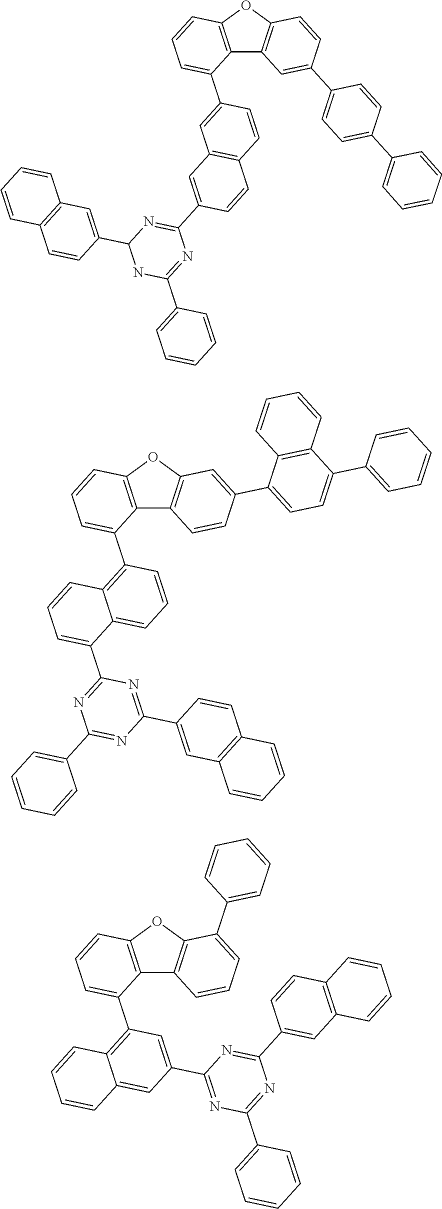

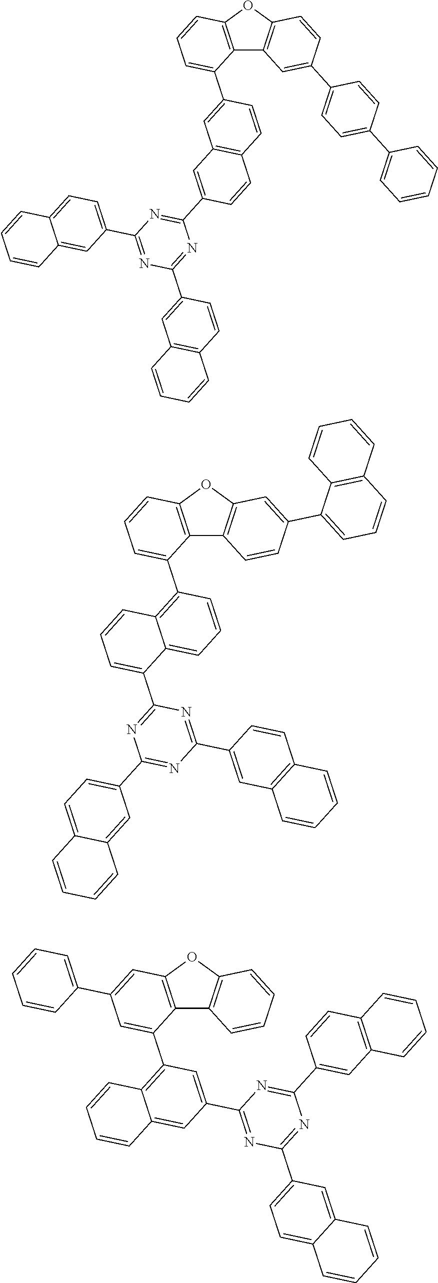 [SMILES: c1ccc(-c2ccc(-c3ccc4oc5cccc(-c6ccc7ccc(-c8nc(-c9ccc%10ccccc%10c9)nc(-c9ccc%10ccccc%10c9)n8)cc7c6)c5c4c3)cc2)cc1 ; c1ccc2cc(-c3nc(-c4ccc5ccccc5c4)nc(-c4cccc5c(-c6cccc7oc8cc(-c9cccc%10ccccc9%10)ccc8c67)cccc45)n3)ccc2c1 ; c1ccc(-c2cc(-c3cc(-c4nc(-c5ccc6ccccc6c5)nc(-c5ccc6ccccc6c5)n4)cc4ccccc34)c3c(c2)oc2ccccc23)cc1]

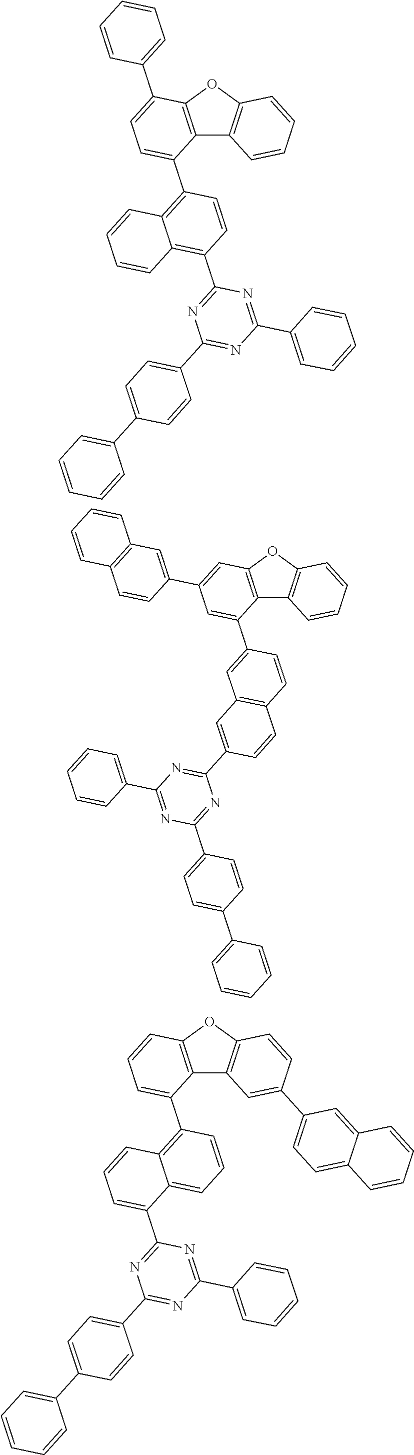

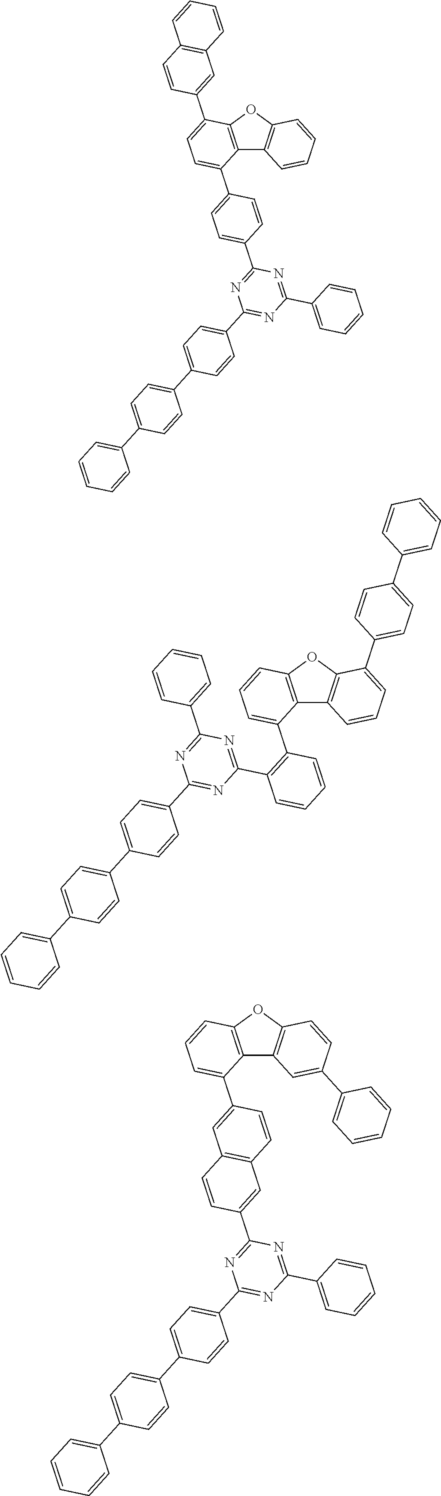 [SMILES: c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1]

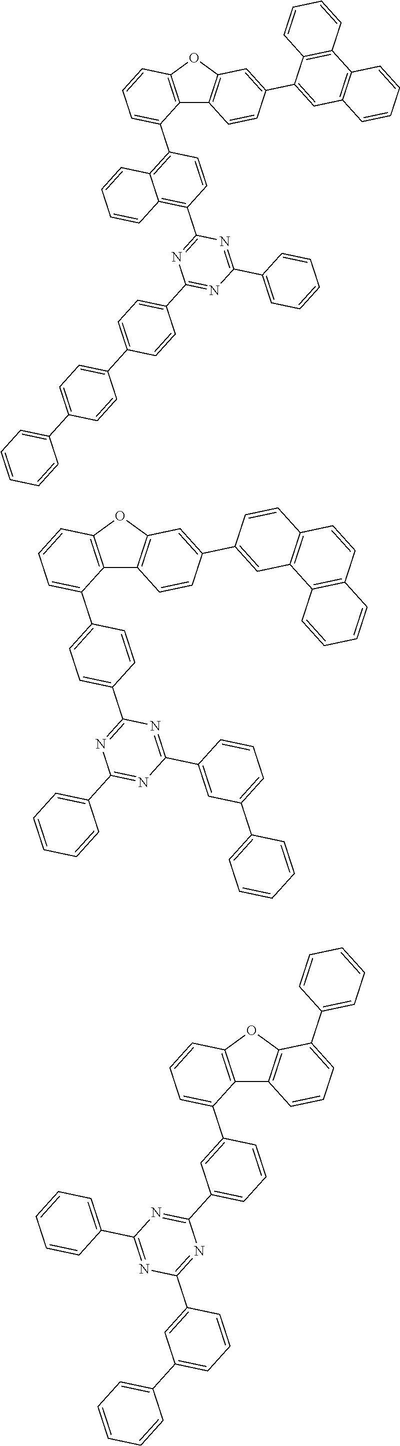 [SMILES: c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1 ; c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1 ; c1ccc(-c2cccc(-c3nc(-c4ccccc4)nc(-c4cccc(-c5cccc6oc7c(-c8ccccc8)cccc7c56)c4)n3)c2)cc1]

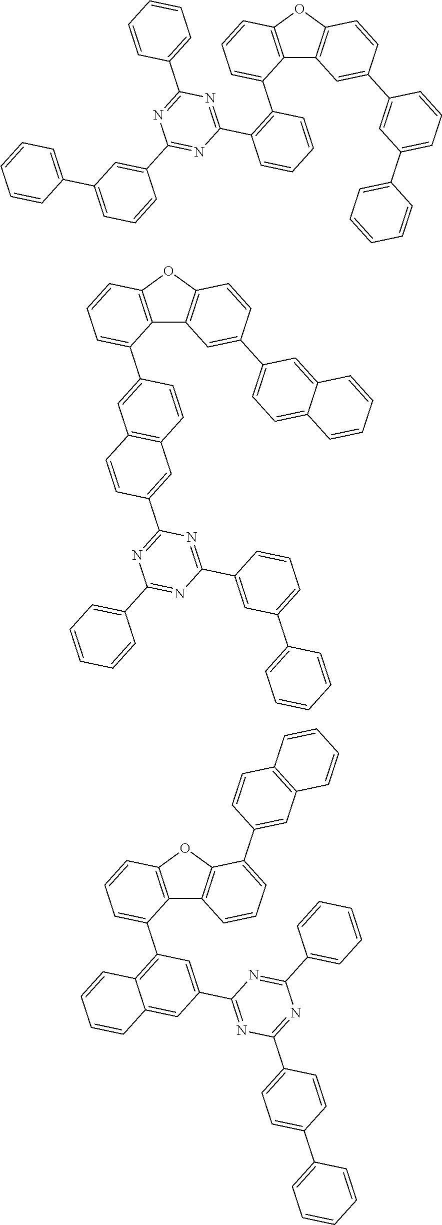

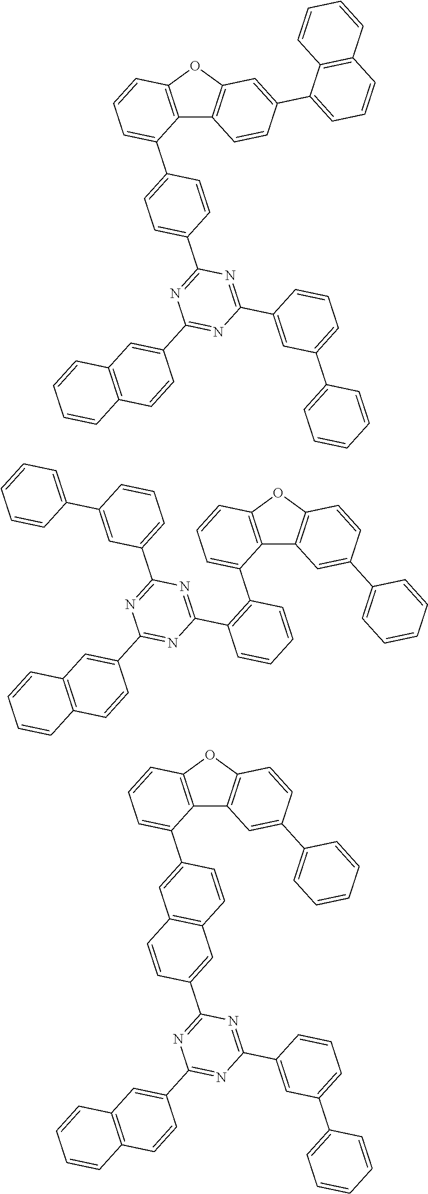 [SMILES: c1ccc(-c2cccc(-c3nc(-c4ccc(-c5cccc6oc7cc(-c8cccc9ccccc89)ccc7c56)cc4)nc(-c4ccc5ccccc5c4)n3)c2)cc1 ; c1ccc(-c2cccc(-c3nc(-c4ccc5ccccc5c4)nc(-c4ccccc4-c4cccc5oc6ccc(-c7ccccc7)cc6c45)n3)c2)cc1 ; c1ccc(-c2cccc(-c3nc(-c4ccc5ccccc5c4)nc(-c4ccc5cc(-c6cccc7oc8ccc(-c9ccccc9)cc8c67)ccc5c4)n3)c2)cc1]

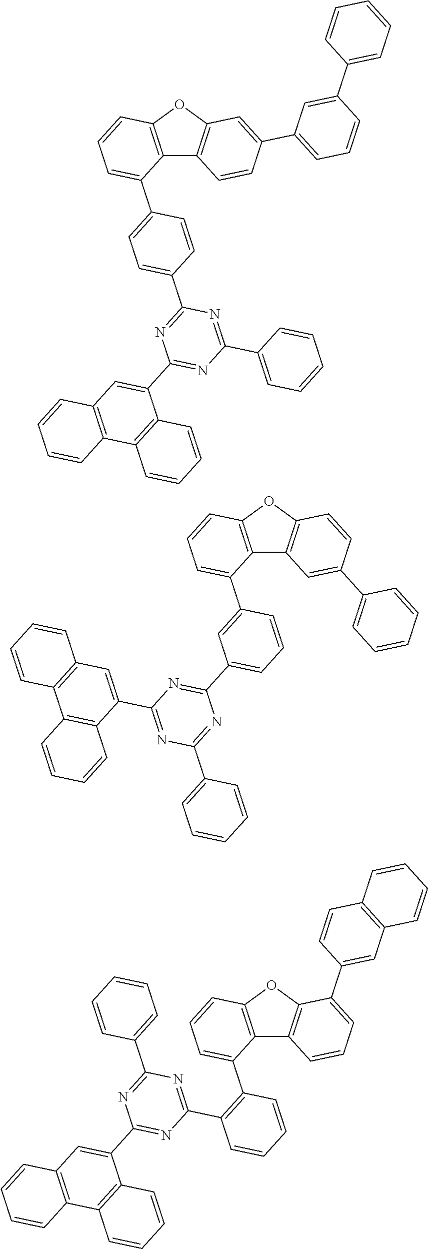

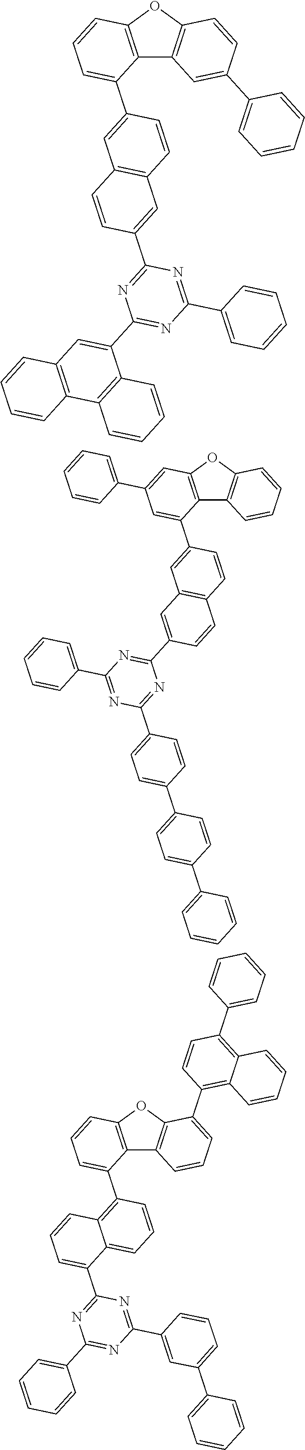

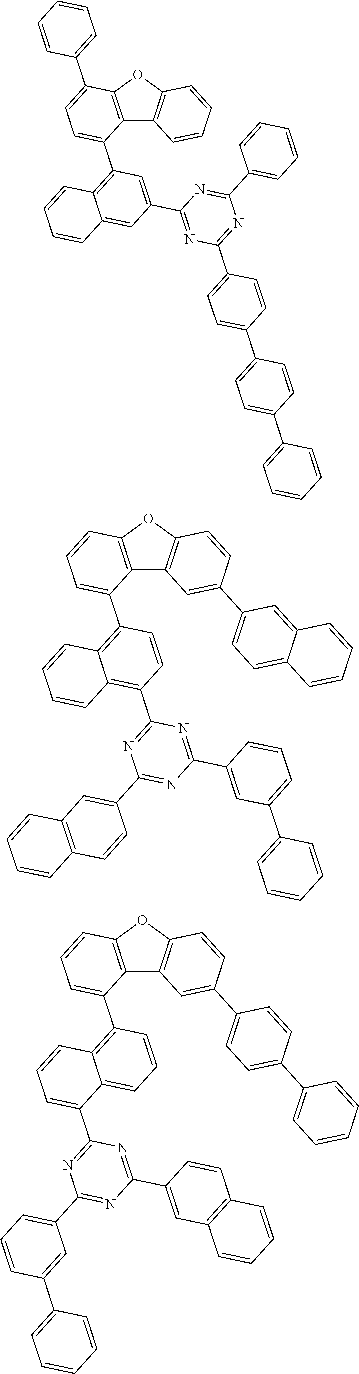 [SMILES: c1ccc(-c2ccc(-c3ccc(-c4nc(-c5ccccc5)nc(-c5cc(-c6ccc(-c7ccccc7)c7oc8ccccc8c67)c6ccccc6c5)n4)cc3)cc2)cc1 ; c1ccc(-c2cccc(-c3nc(-c4ccc5ccccc5c4)nc(-c4ccc(-c5cccc6oc7ccc(-c8ccc9ccccc9c8)cc7c56)c5ccccc45)n3)c2)cc1 ; c1ccc(-c2ccc(-c3ccc4oc5cccc(-c6cccc7c(-c8nc(-c9cccc(-c%10ccccc%10)c9)nc(-c9ccc%10ccccc%10c9)n8)cccc67)c5c4c3)cc2)cc1]

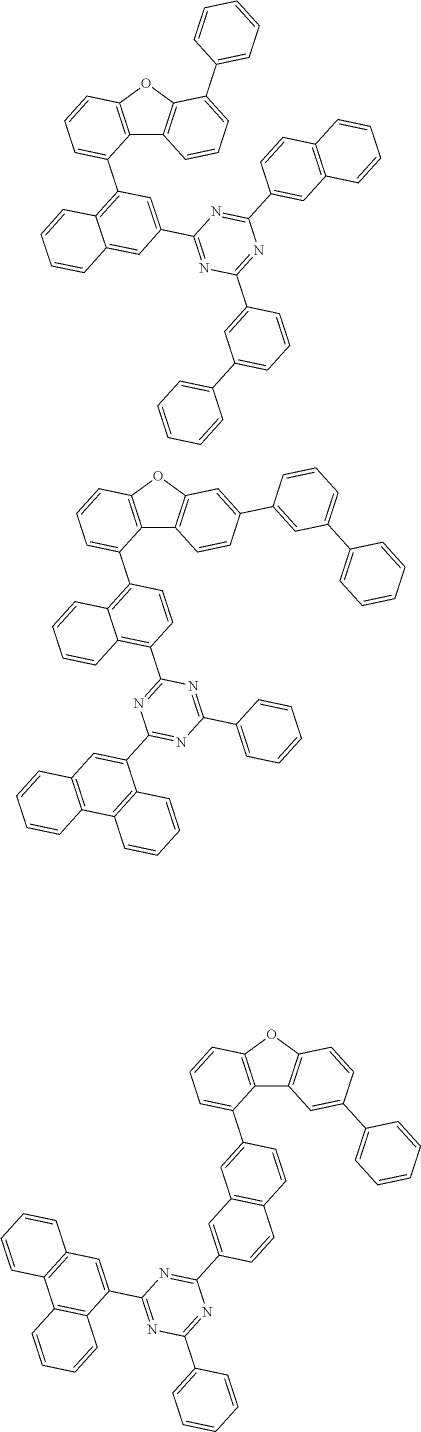

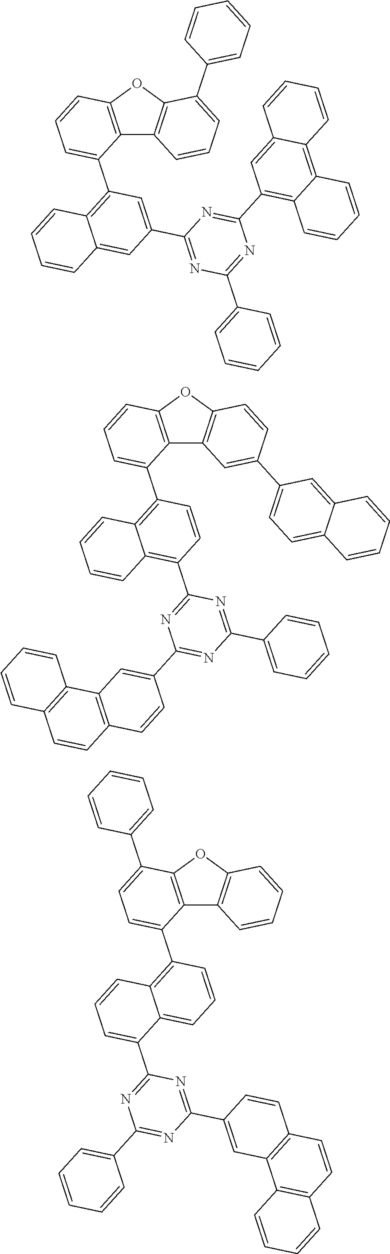

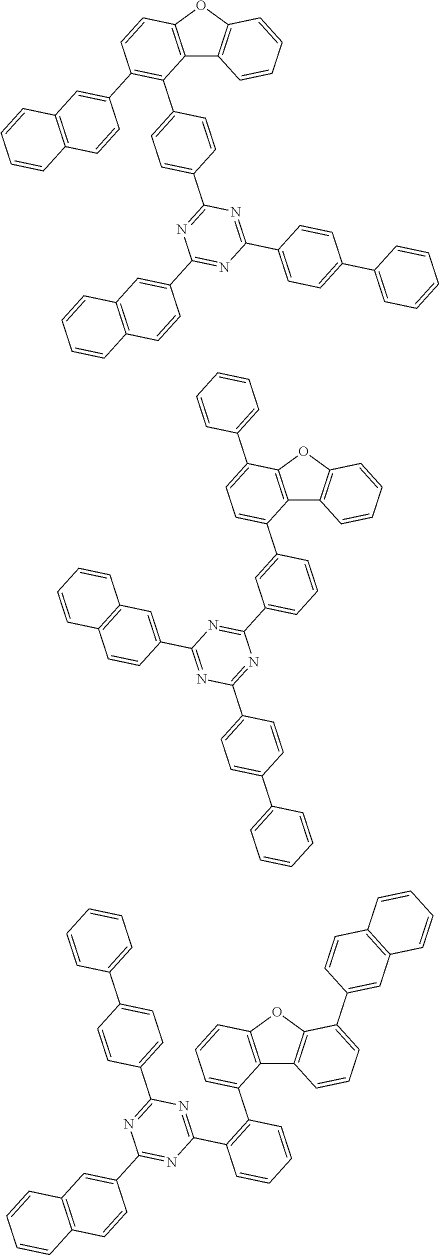 [SMILES: c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1 ; c1ccc(-c2ccc(-c3nc(-c4cccc(-c5ccc(-c6ccccc6)c6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

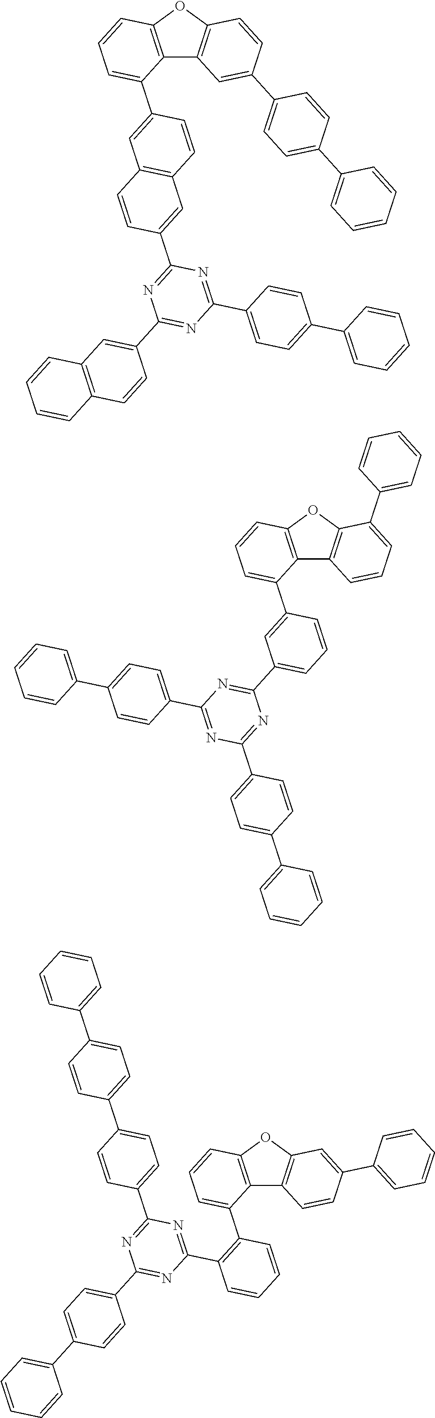

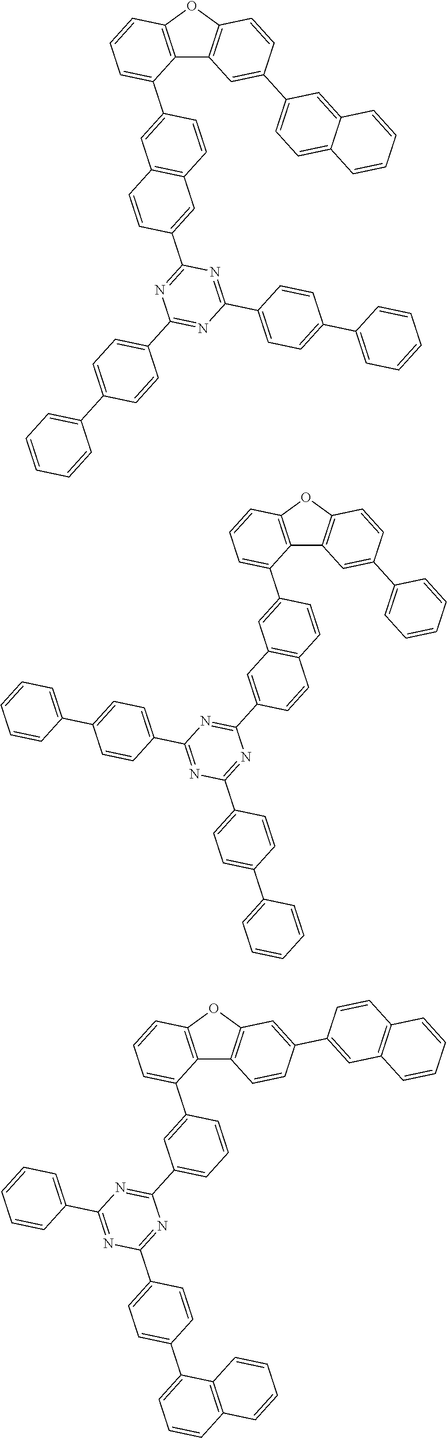 [SMILES: c1ccc(-c2ccc(-c3nc(-c4ccc(-c5ccccc5)cc4)nc(-c4ccc5cc(-c6cccc7oc8ccc(-c9ccc%10ccccc%10c9)cc8c67)ccc5c4)n3)cc2)cc1 ; c1ccc(-c2ccc(-c3nc(-c4ccc(-c5ccccc5)cc4)nc(-c4ccc5ccc(-c6cccc7oc8ccc(-c9ccccc9)cc8c67)cc5c4)n3)cc2)cc1 ; c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1]

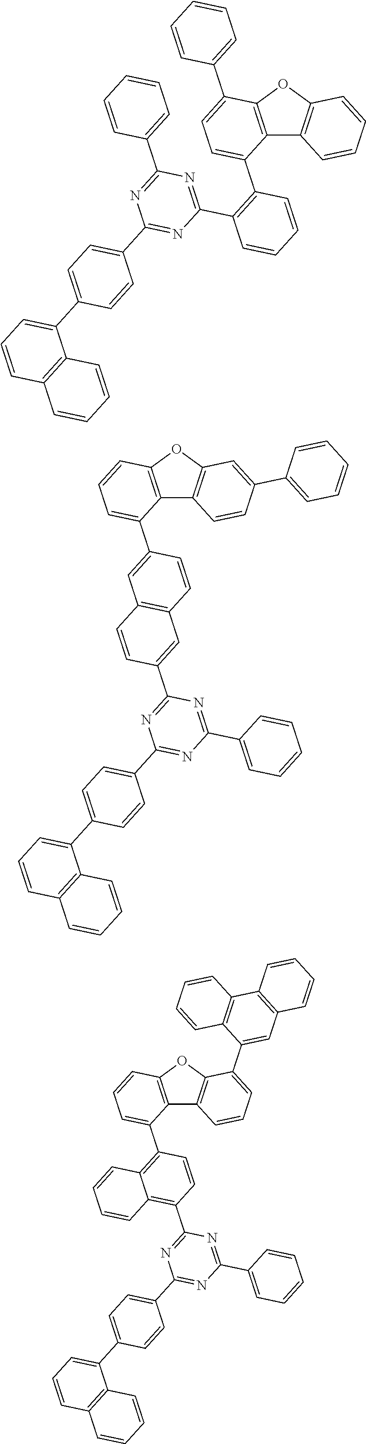 [SMILES: c1ccc(-c2nc(-c3ccc(-c4cccc5ccccc45)cc3)nc(-c3ccccc3-c3ccc(-c4ccccc4)c4oc5ccccc5c34)n2)cc1 ; c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1 ; c1ccc(-c2nc(-c3ccc(-c4cccc5ccccc45)cc3)nc(-c3ccc(-c4cccc5oc6c(-c7cc8ccccc8c8ccccc78)cccc6c45)c4ccccc34)n2)cc1]

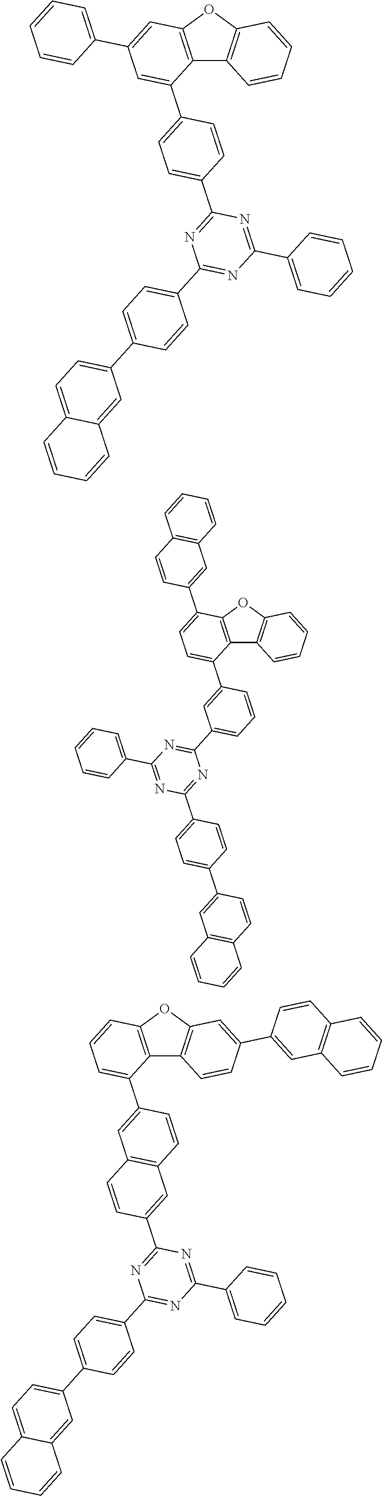

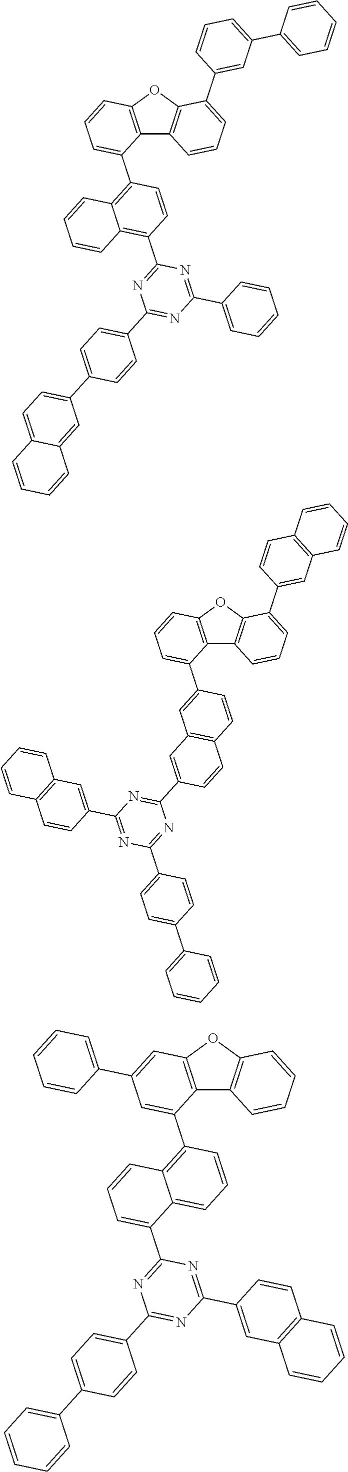

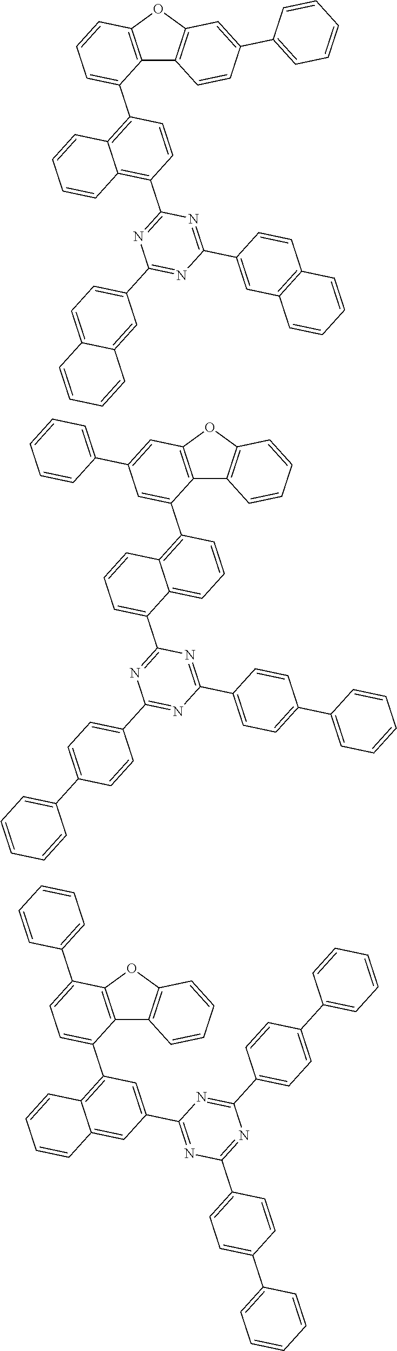

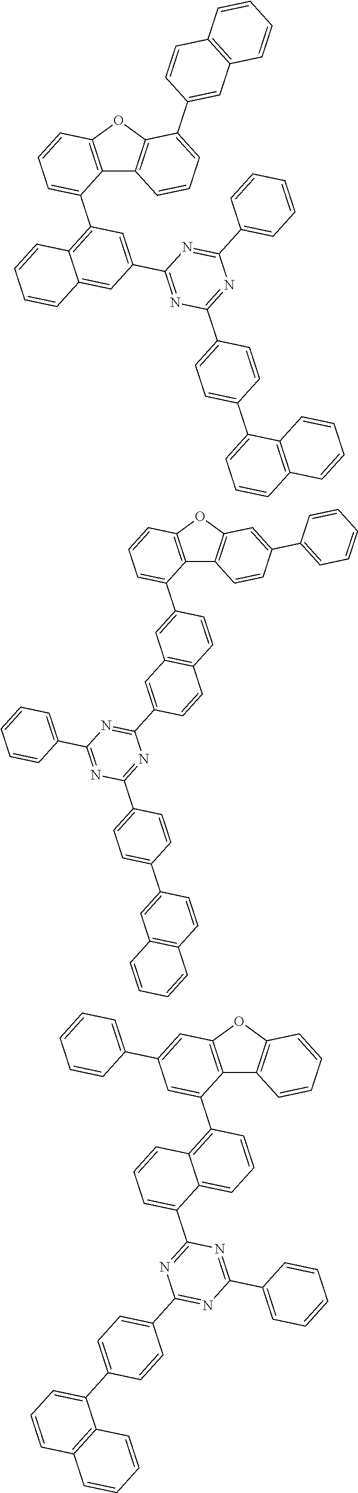

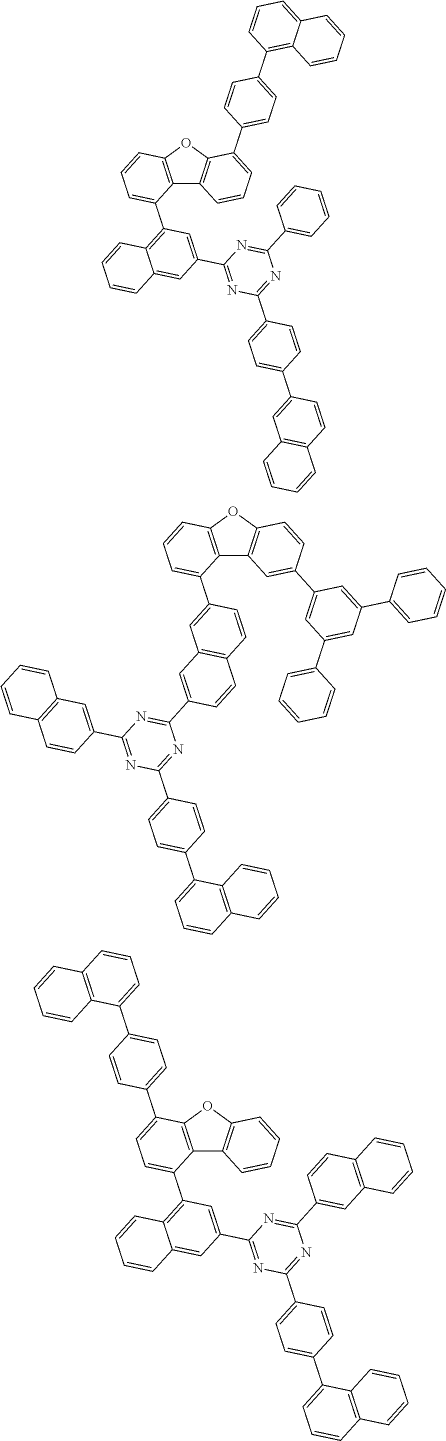

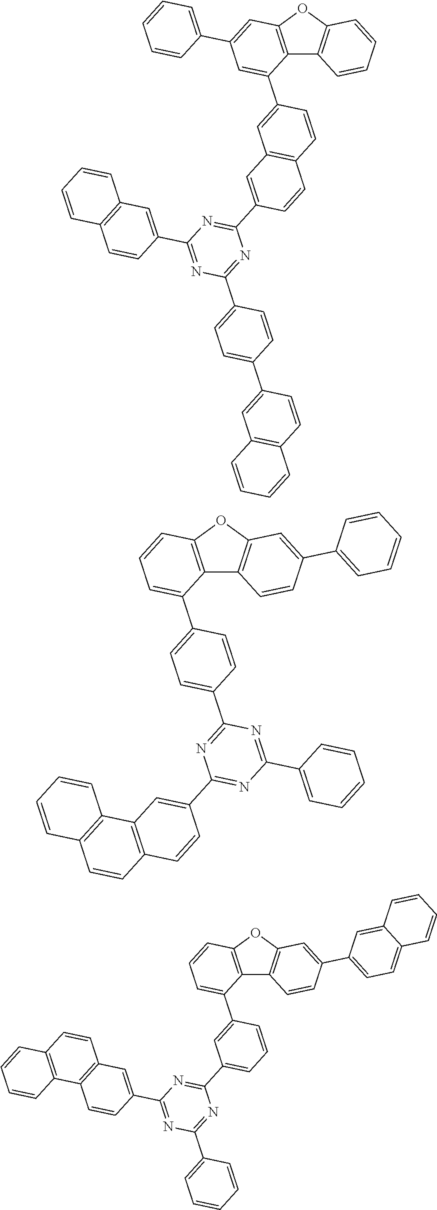

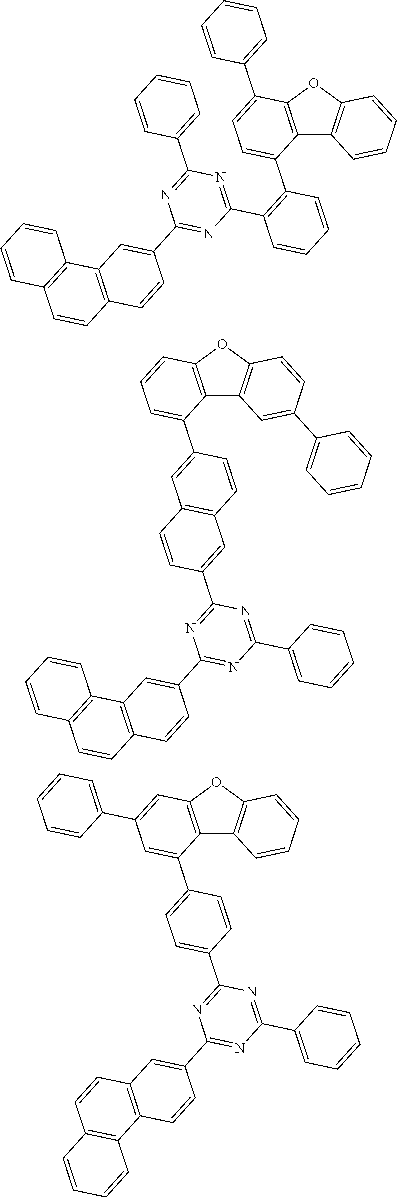

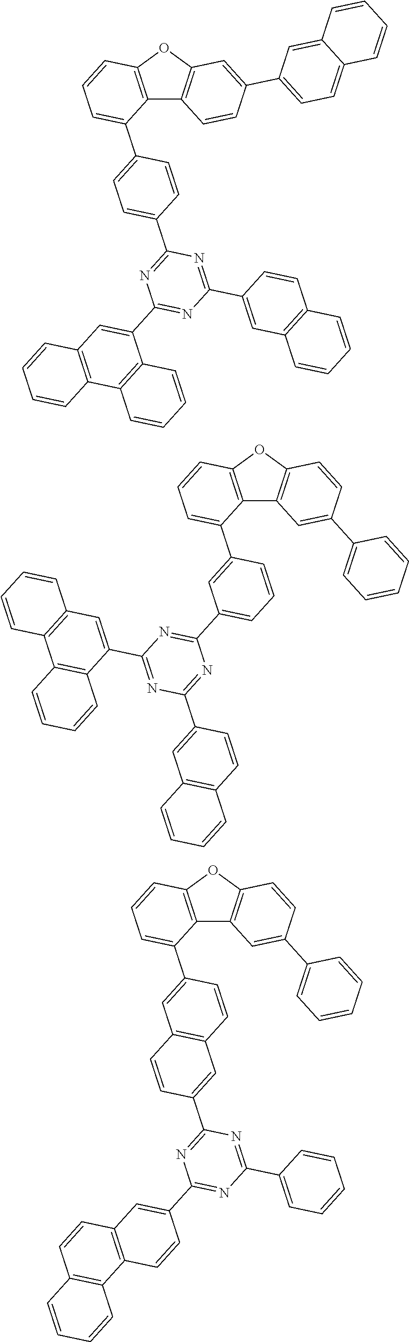

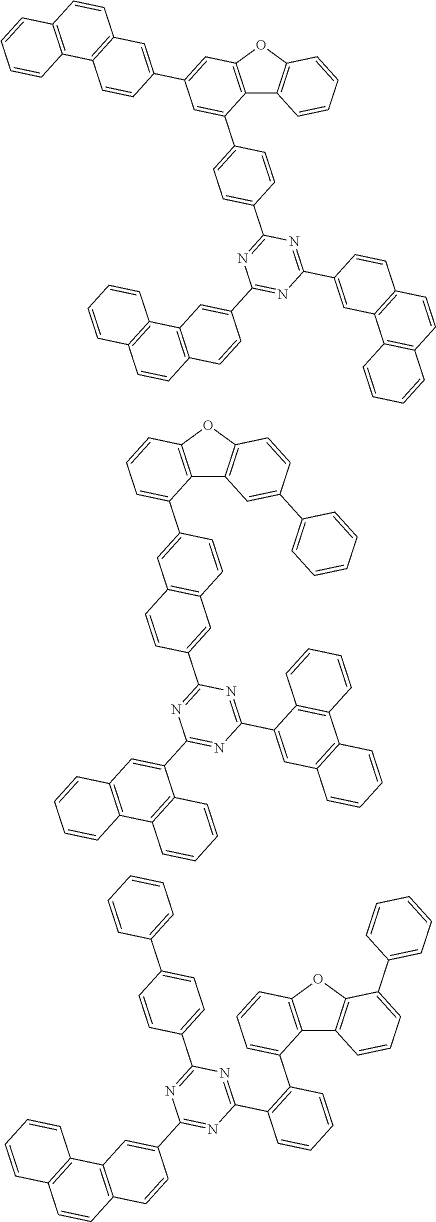

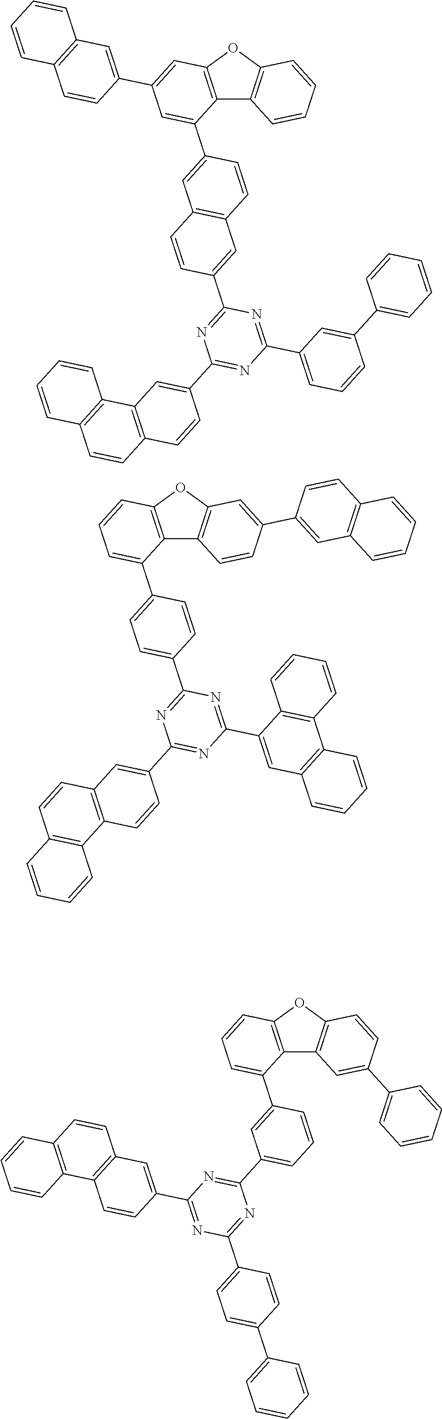

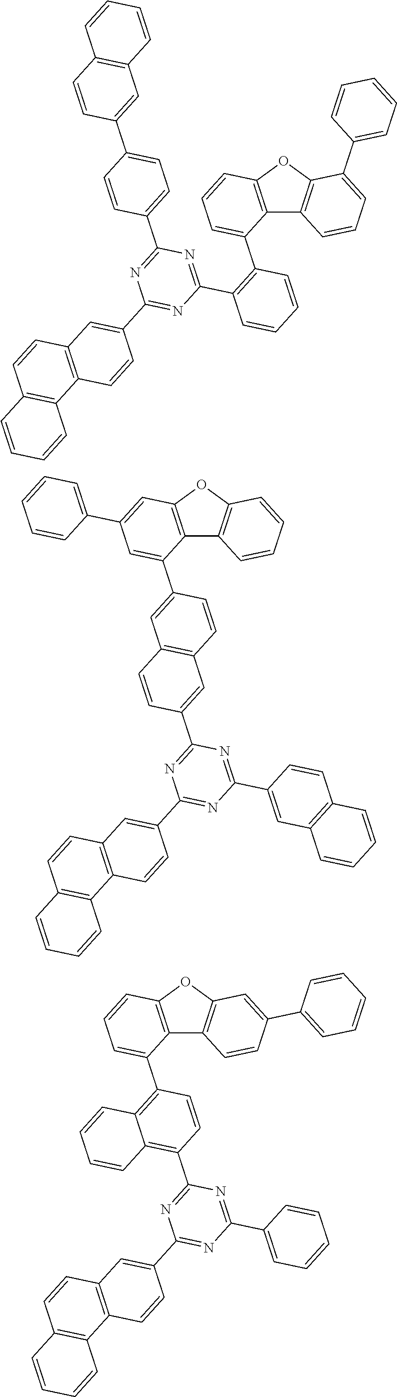

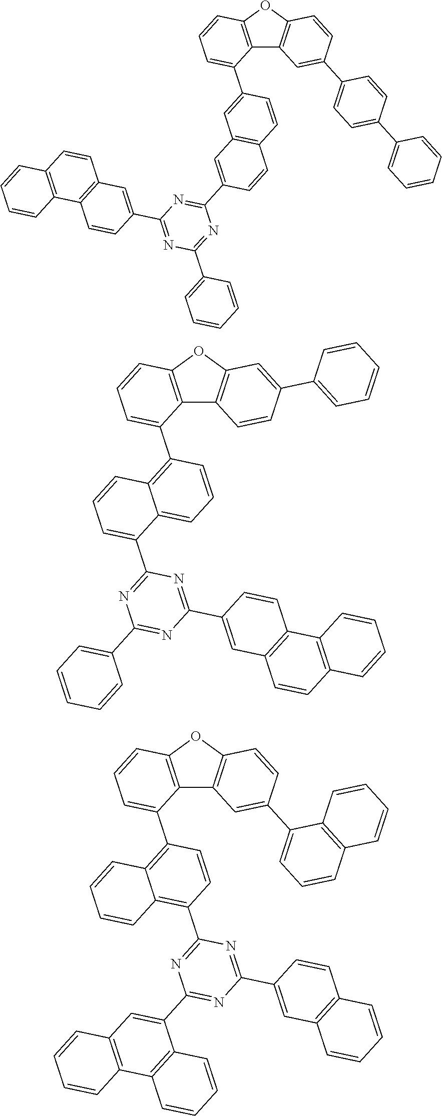

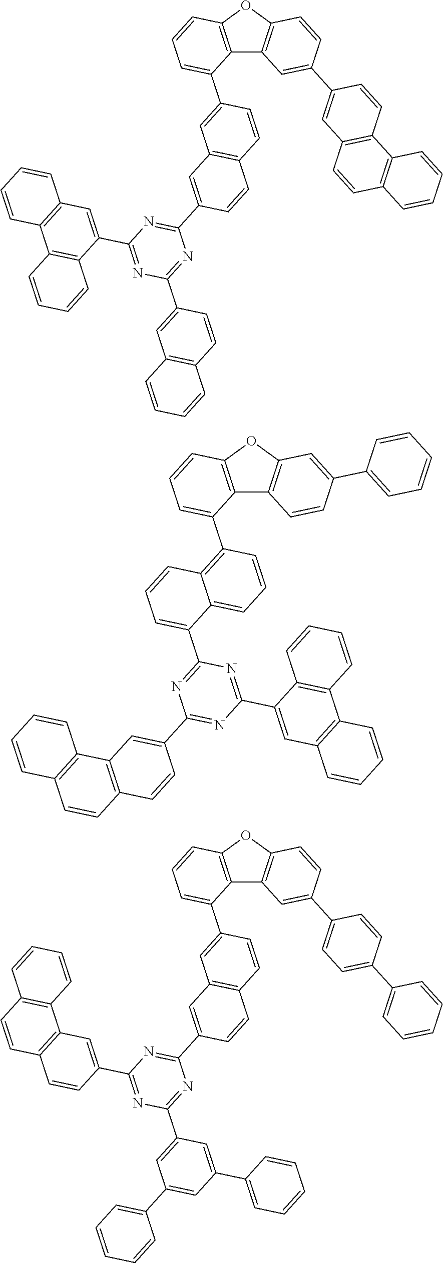

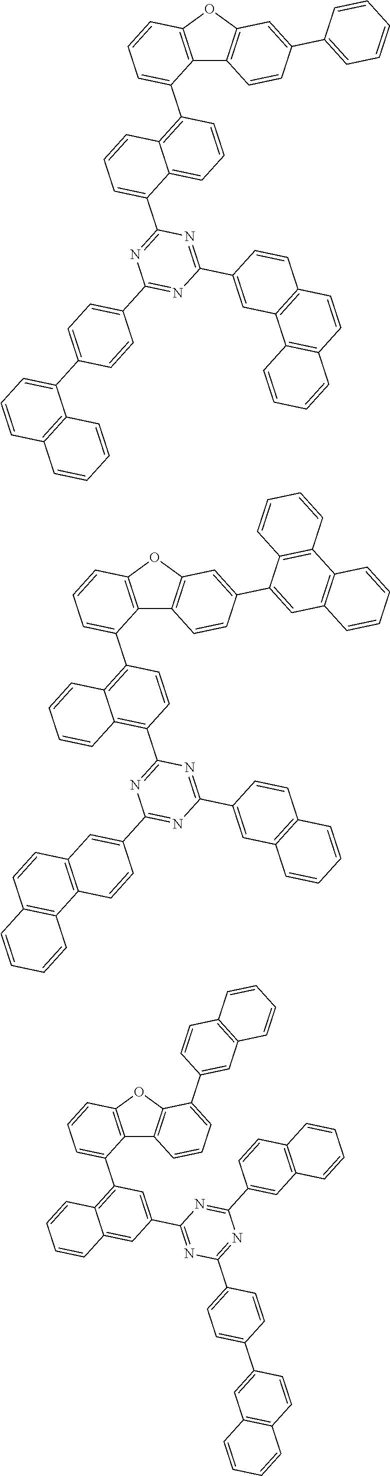 [SMILES: c1ccc(-c2ccc3c(c2)oc2cccc(-c4cccc5c(-c6nc(-c7ccc(-c8cccc9ccccc89)cc7)nc(-c7ccc8ccc9ccccc9c8c7)n6)cccc45)c23)cc1 ; c1ccc2cc(-c3nc(-c4ccc5c(ccc6ccccc65)c4)nc(-c4ccc(-c5cccc6oc7cc(-c8cc9ccccc9c9ccccc89)ccc7c56)c5ccccc45)n3)ccc2c1 ; c1ccc2cc(-c3ccc(-c4nc(-c5ccc6ccccc6c5)nc(-c5cc(-c6cccc7oc8c(-c9ccc%10ccccc%10c9)cccc8c67)c6ccccc6c5)n4)cc3)ccc2c1]

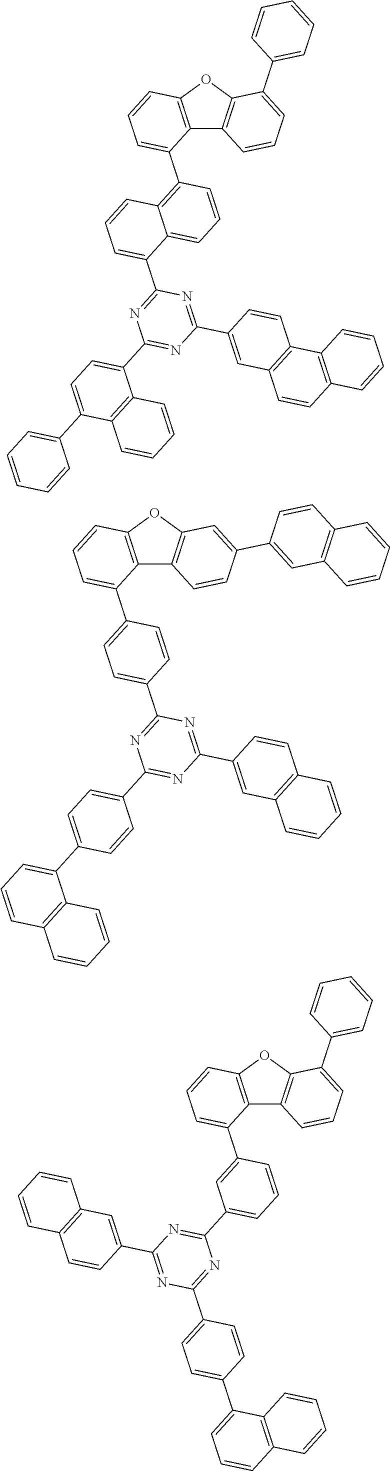 [SMILES: c1ccc(-c2ccc(-c3nc(-c4ccc5c(ccc6ccccc65)c4)nc(-c4cccc5c(-c6cccc7oc8c(-c9ccccc9)cccc8c67)cccc45)n3)c3ccccc23)cc1 ; c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1 ; c1ccc(-c2cccc3c2oc2cccc(-c4cccc(-c5nc(-c6ccc(-c7cccc8ccccc78)cc6)nc(-c6ccc7ccccc7c6)n5)c4)c23)cc1]

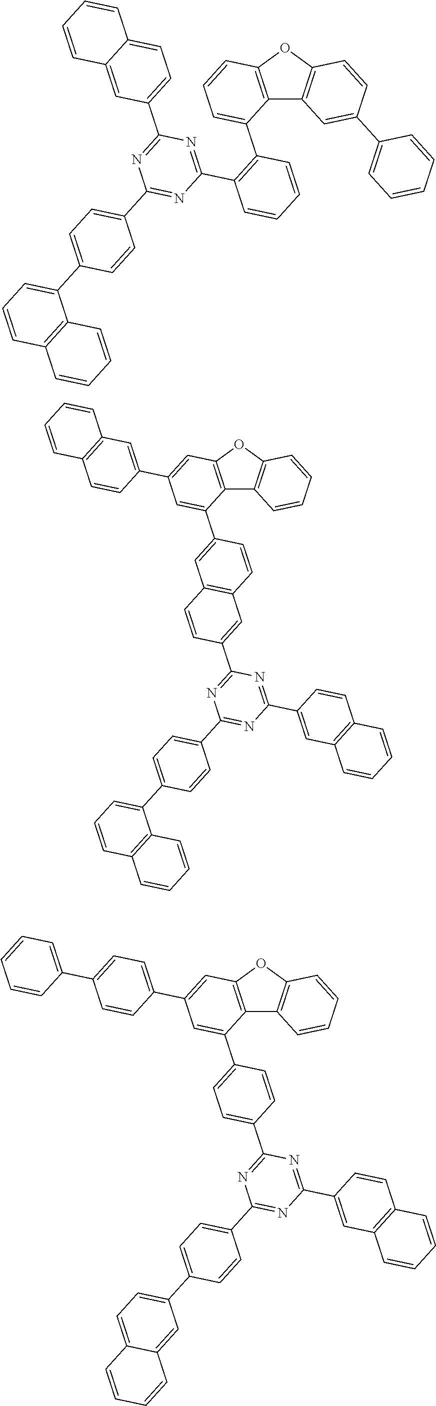

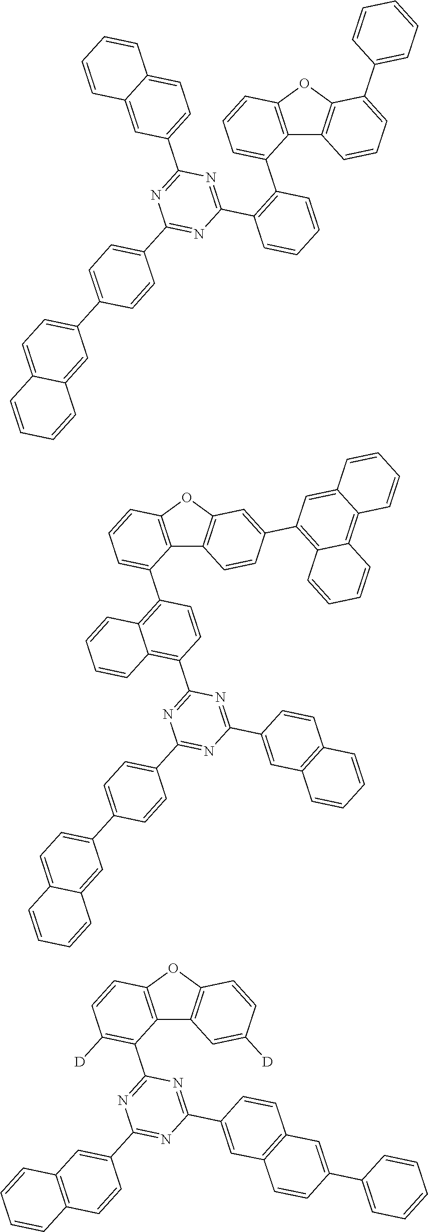 [SMILES: c1ccc(-c2cccc3c2oc2cccc(-c4ccccc4-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1 ; [2H]c1ccc2oc3ccc([2H])c(-c4nc(-c5ccc6ccccc6c5)nc(-c5ccc6cc(-c7ccccc7)ccc6c5)n4)c3c2c1]

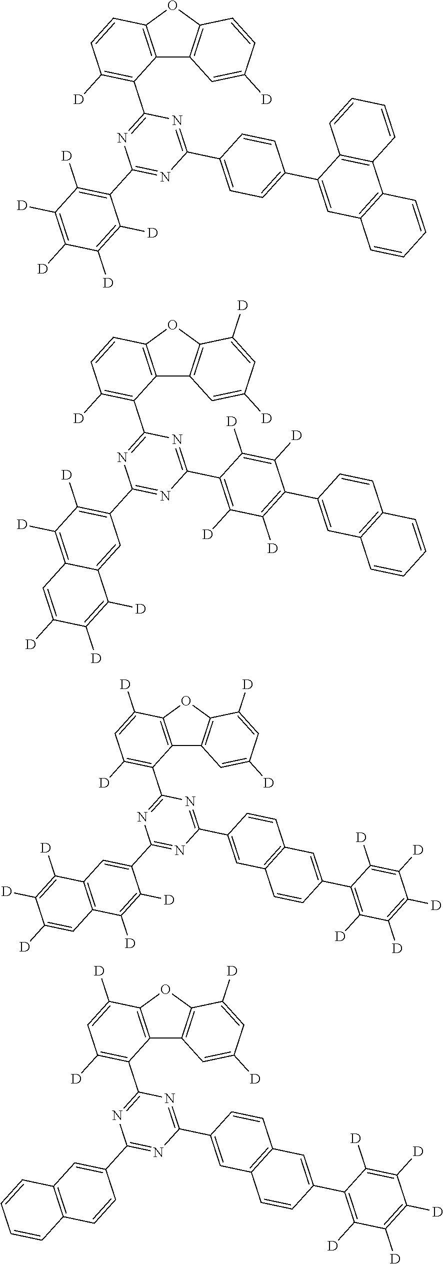

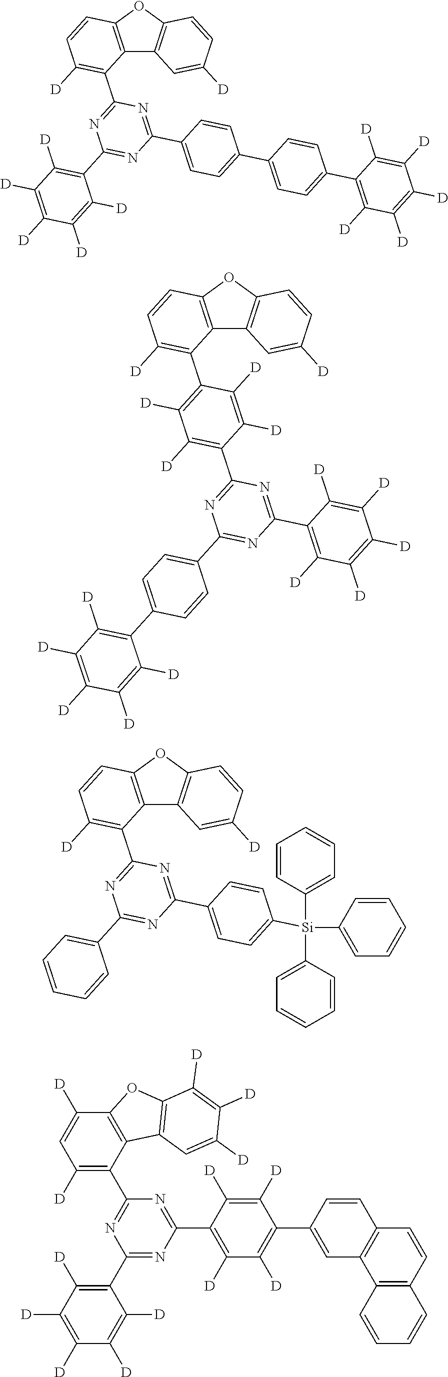

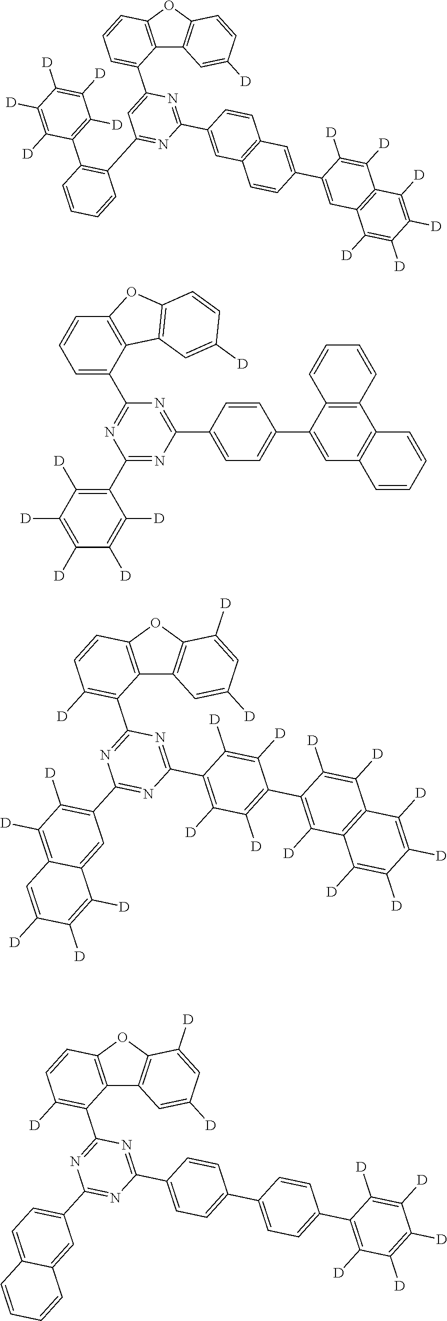

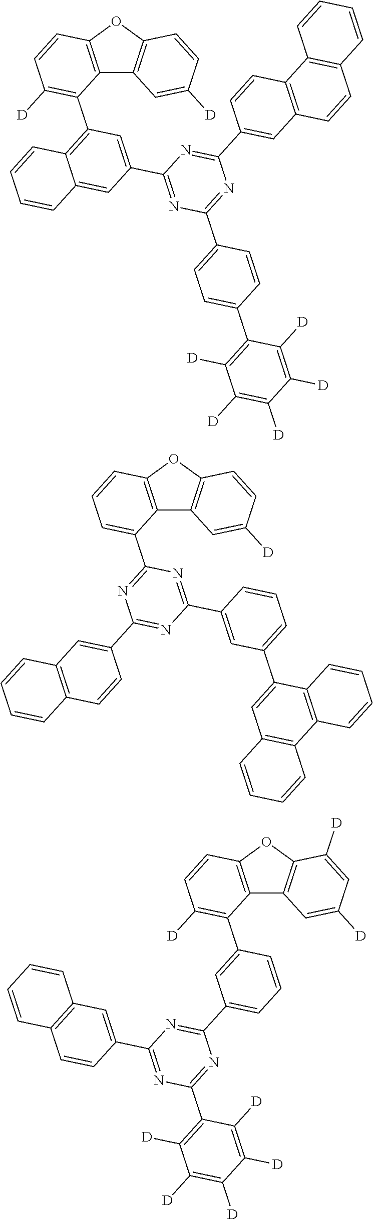

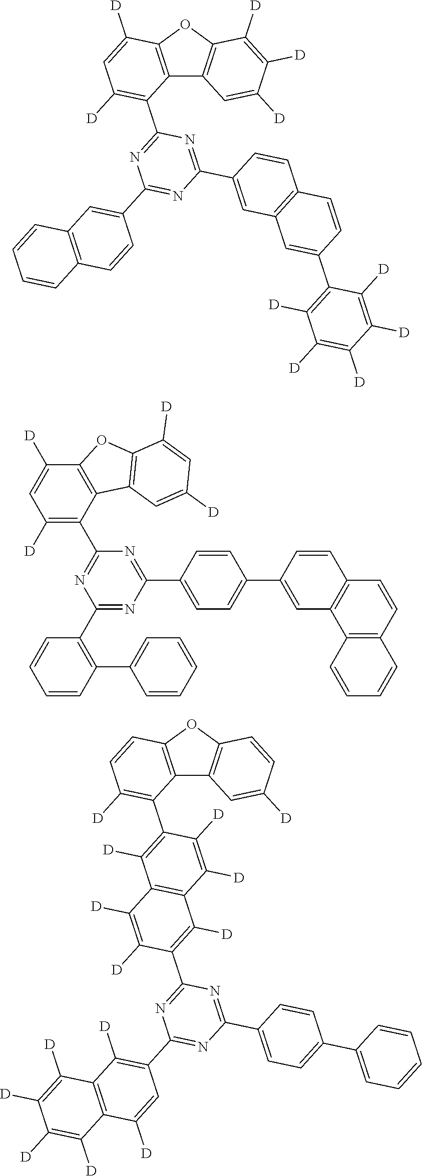

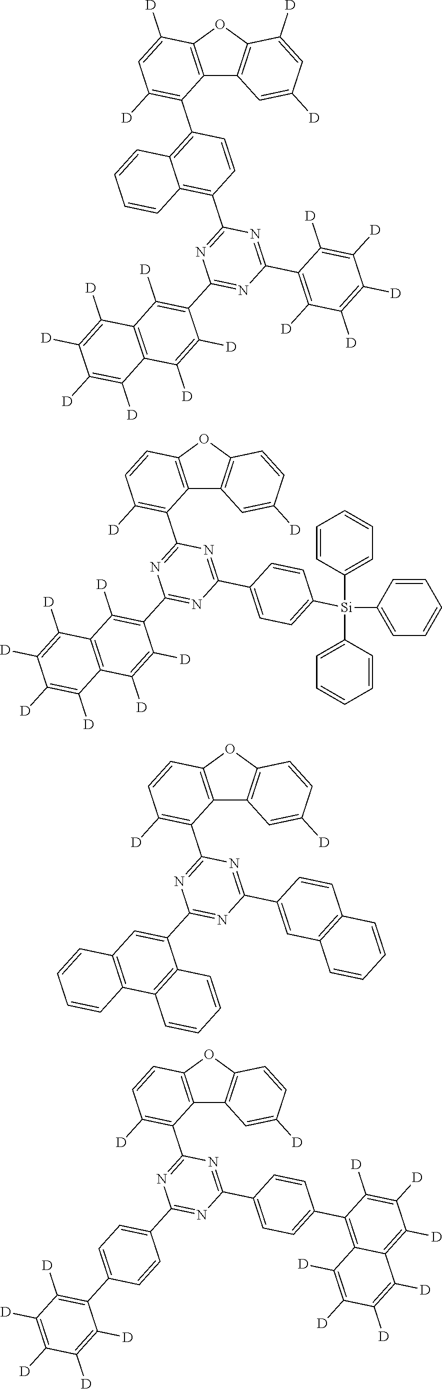

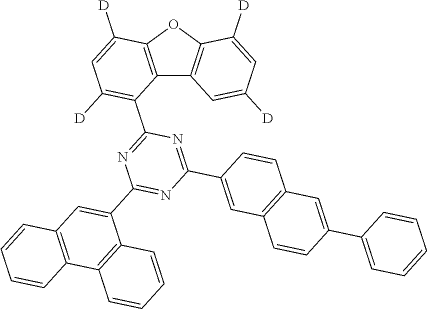 [SMILES: [2H]c1cc([2H])c2oc3c([2H])cc([2H])c(-c4nc(-c5ccc6cc(-c7ccccc7)ccc6c5)nc(-c5cc6ccccc6c6ccccc56)n4)c3c2c1]

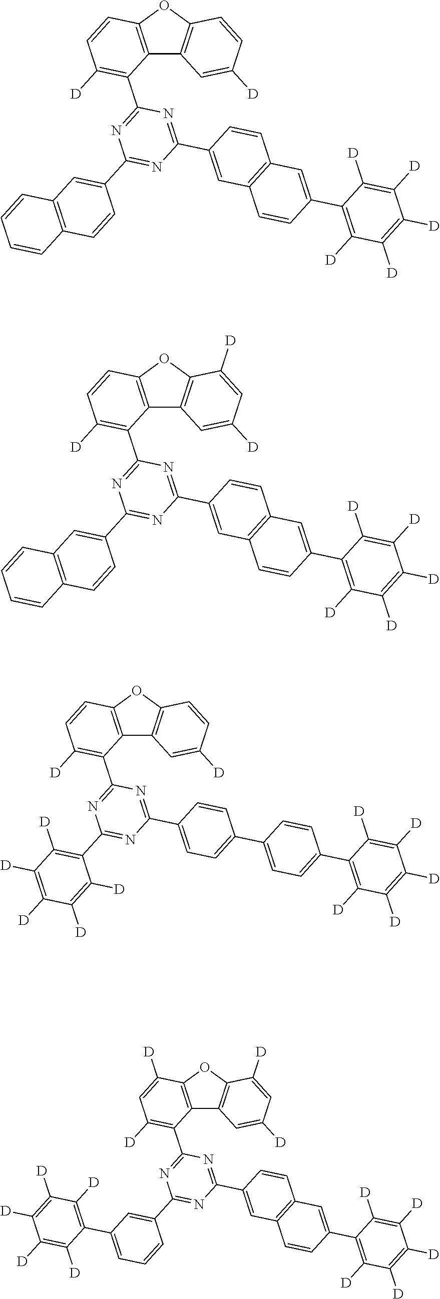

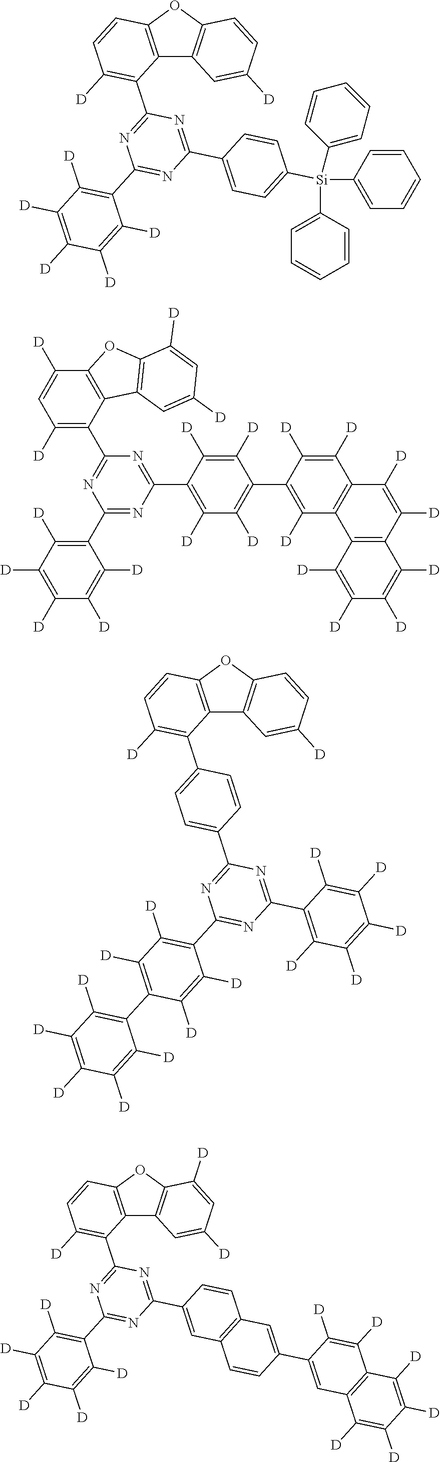

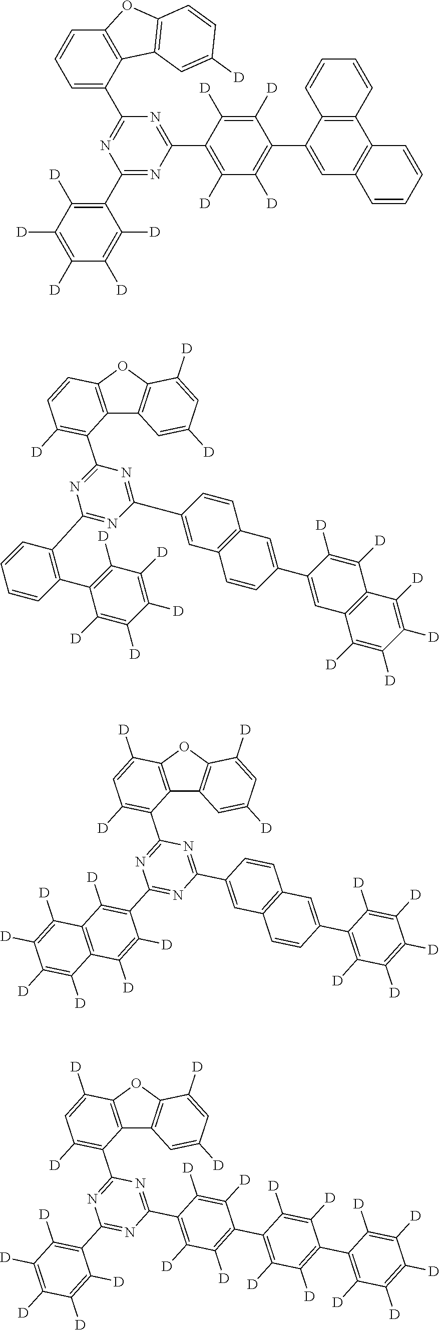

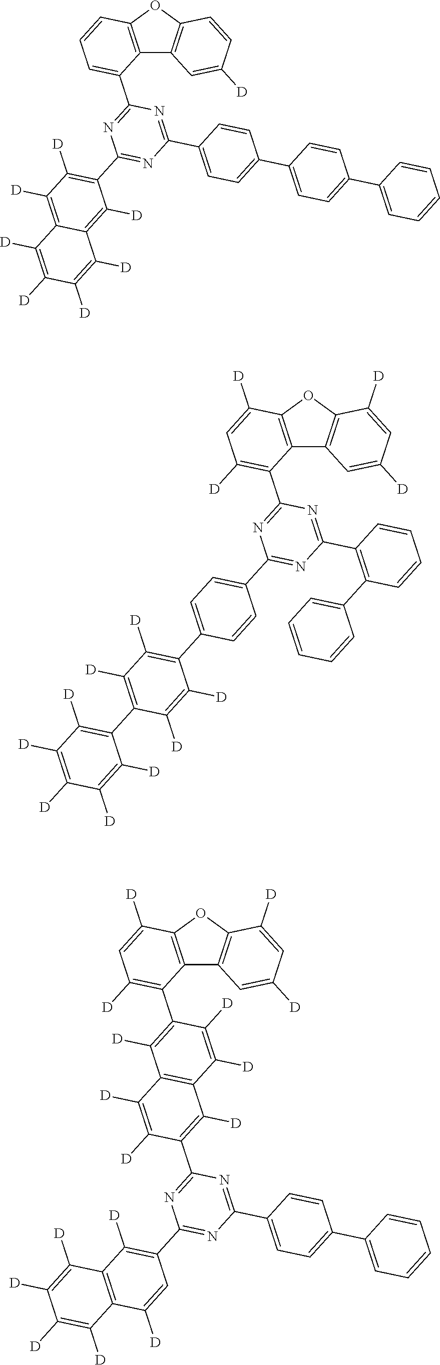 [SMILES: [2H]c1ccc2oc3cccc(-c4nc(-c5ccc(-c6ccc(-c7ccccc7)cc6)cc5)nc(-c5c([2H])c([2H])c6c([2H])c([2H])c([2H])c([2H])c6c5[2H])n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccccc5-c5ccccc5)n4)c3c2c1 ; [2H]c1cc([2H])c2oc3c([2H])cc([2H])c(-c4c([2H])c([2H])c5c([2H])c(-c6nc(-c7ccc(-c8ccccc8)cc7)nc(-c7cc([2H])c8c([2H])c([2H])c([2H])c([2H])c8c7[2H])n6)c([2H])c([2H])c5c4[2H])c3c2c1]

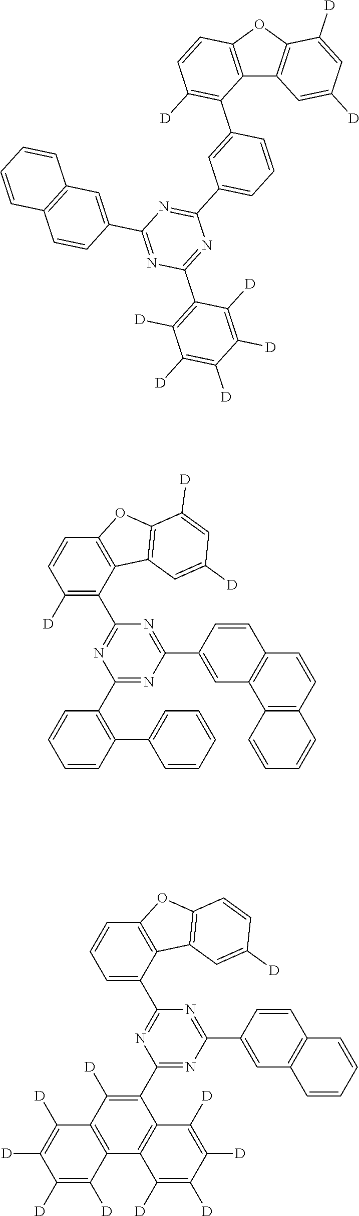 [SMILES: [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1 ; [2H]c1cc([2H])c2oc3ccc([2H])c(-c4nc(-c5ccc6ccc7ccccc7c6c5)nc(-c5ccccc5-c5ccccc5)n4)c3c2c1 ; [2H]c1ccc2oc3cccc(-c4nc(-c5ccc6ccccc6c5)nc(-c5c([2H])c6c([2H])c([2H])c([2H])c([2H])c6c6c([2H])c([2H])c([2H])c([2H])c56)n4)c3c2c1]

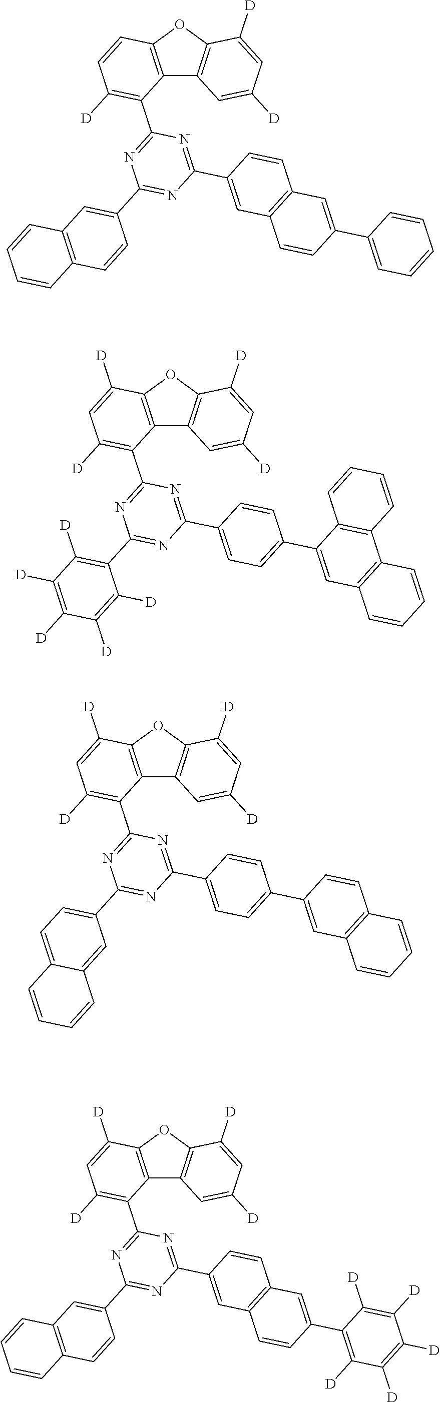 [SMILES: [2H]c1cc([2H])c2oc3ccc([2H])c(-c4nc(-c5ccc6ccccc6c5)nc(-c5ccc6cc(-c7ccccc7)ccc6c5)n4)c3c2c1 ; [2H]c1cc([2H])c2oc3c([2H])cc([2H])c(-c4nc(-c5ccc(-c6cc7ccccc7c7ccccc67)cc5)nc(-c5c([2H])c([2H])c([2H])c([2H])c5[2H])n4)c3c2c1 ; [2H]c1cc([2H])c2oc3c([2H])cc([2H])c(-c4nc(-c5ccc(-c6ccc7ccccc7c6)cc5)nc(-c5ccc6ccccc6c5)n4)c3c2c1 ; [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1]

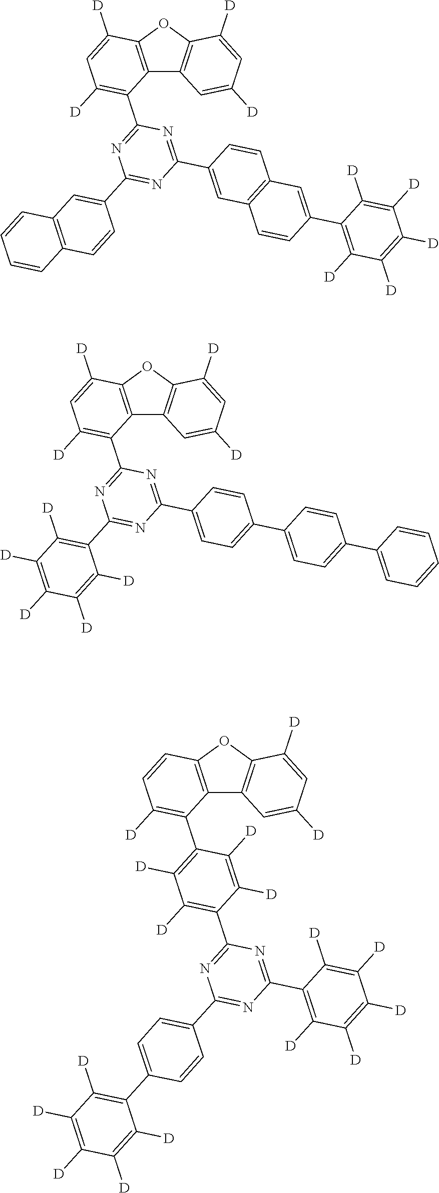 [SMILES: [2H]c1cc([2H])c2oc3c([2H])cc([2H])c(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7ccccc7)cc6)cc5)nc(-c5c([2H])c([2H])c([2H])c([2H])c5[2H])n4)c3c2c1 ; [2H]c1cc([2H])c2oc3ccc([2H])c(-c4c([2H])c([2H])c(-c5nc(-c6ccc(-c7c([2H])c([2H])c([2H])c([2H])c7[2H])cc6)nc(-c6c([2H])c([2H])c([2H])c([2H])c6[2H])n5)c([2H])c4[2H])c3c2c1]

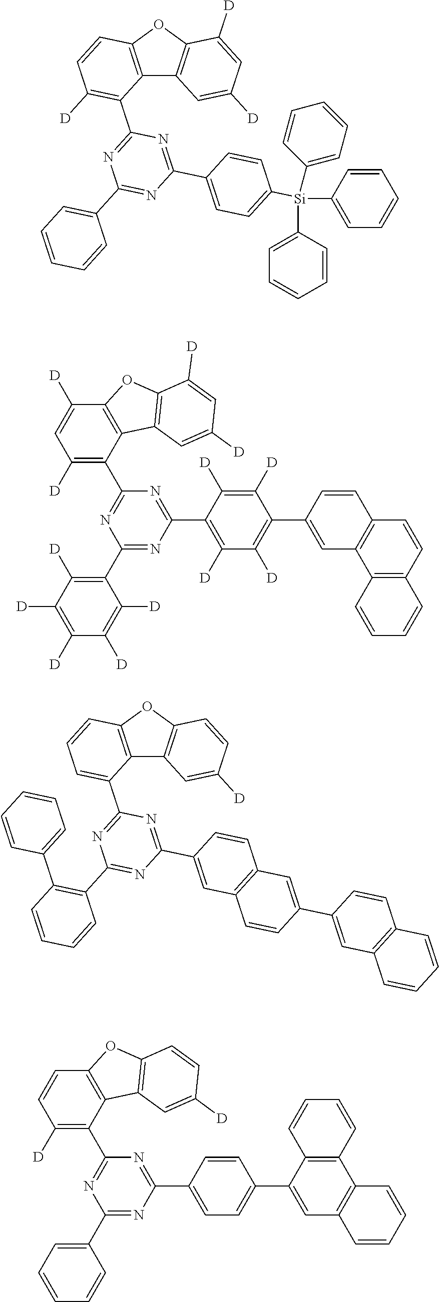 [SMILES: [2H]c1cc([2H])c2oc3ccc([2H])c(-c4nc(-c5ccccc5)nc(-c5ccc([Si](c6ccccc6)(c6ccccc6)c6ccccc6)cc5)n4)c3c2c1 ; [2H]c1cc([2H])c2oc3c([2H])cc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1ccc2oc3cccc(-c4nc(-c5ccc6cc(-c7ccc8ccccc8c7)ccc6c5)nc(-c5ccccc5-c5ccccc5)n4)c3c2c1 ; [2H]c1ccc2oc3ccc([2H])c(-c4nc(-c5ccccc5)nc(-c5ccc(-c6cc7ccccc7c7ccccc67)cc5)n4)c3c2c1]

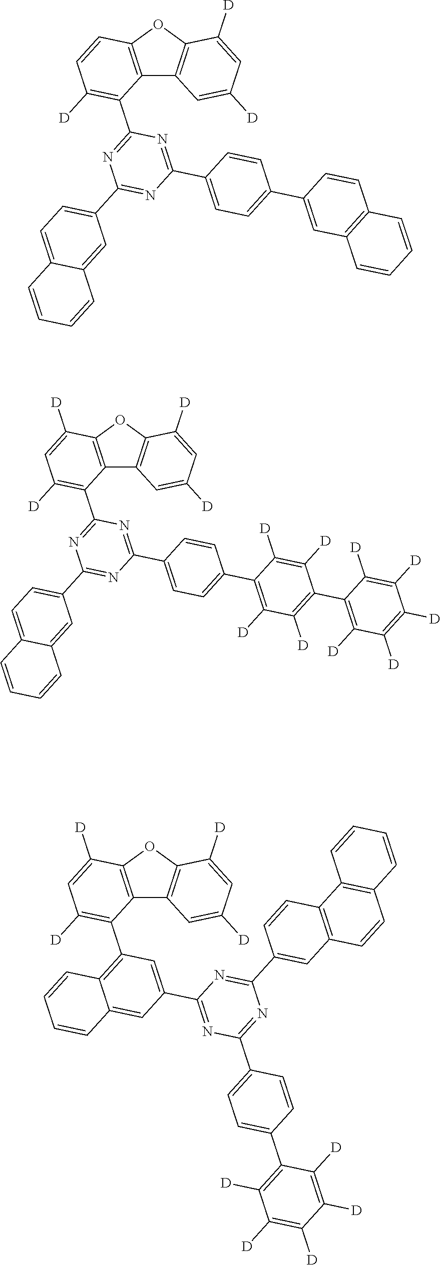 [SMILES: [2H]c1cc([2H])c2oc3ccc([2H])c(-c4nc(-c5ccc(-c6ccc7ccccc7c6)cc5)nc(-c5ccc6ccccc6c5)n4)c3c2c1 ; [2H]c1cc([2H])c2oc3c([2H])cc([2H])c(-c4nc(-c5ccc(-c6c([2H])c([2H])c(-c7c([2H])c([2H])c([2H])c([2H])c7[2H])c([2H])c6[2H])cc5)nc(-c5ccc6ccccc6c5)n4)c3c2c1 ; [2H]c1cc([2H])c2oc3c([2H])cc([2H])c(-c4cc(-c5nc(-c6ccc(-c7c([2H])c([2H])c([2H])c([2H])c7[2H])cc6)nc(-c6ccc7c(ccc8ccccc87)c6)n5)cc5ccccc45)c3c2c1]

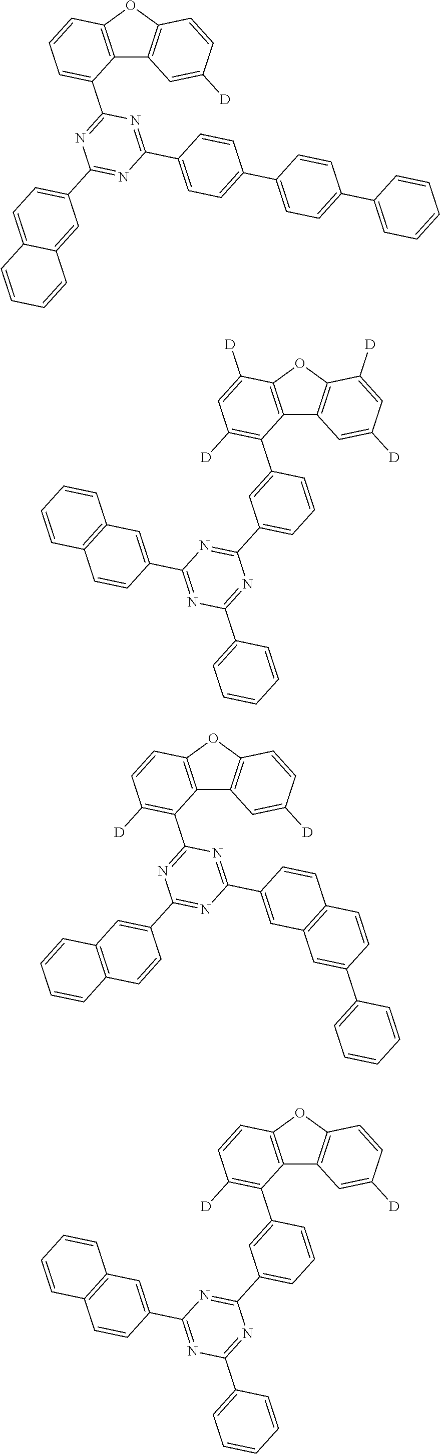

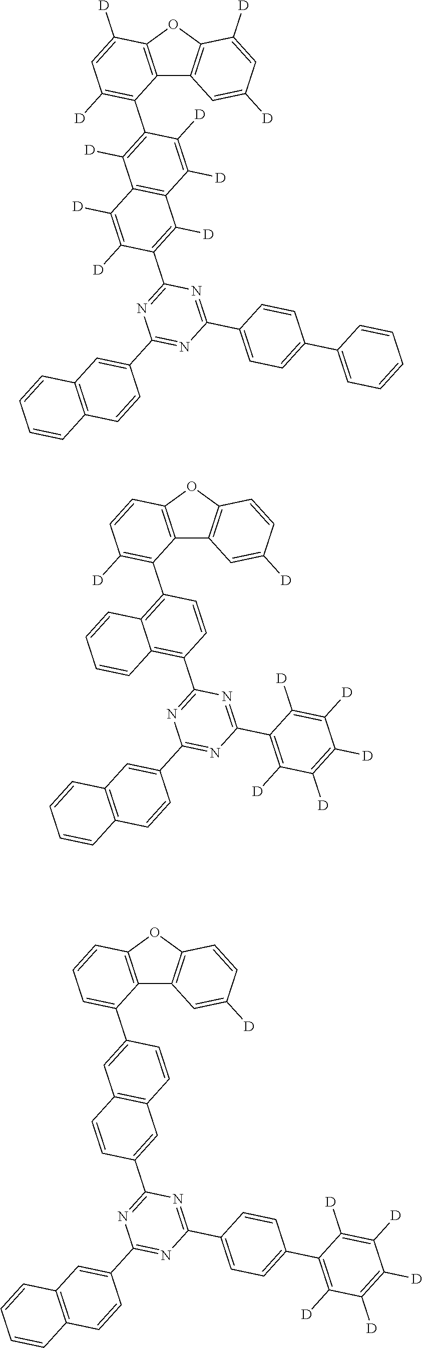 [SMILES: [2H]c1cc([2H])c2oc3c([2H])cc([2H])c(-c4c([2H])c([2H])c5c([2H])c(-c6nc(-c7ccc(-c8ccccc8)cc7)nc(-c7ccc8ccccc8c7)n6)c([2H])c([2H])c5c4[2H])c3c2c1 ; [2H]c1ccc2oc3ccc([2H])c(-c4ccc(-c5nc(-c6ccc7ccccc7c6)nc(-c6c([2H])c([2H])c([2H])c([2H])c6[2H])n5)c5ccccc45)c3c2c1 ; [2H]c1ccc2oc3cccc(-c4ccc5cc(-c6nc(-c7ccc(-c8c([2H])c([2H])c([2H])c([2H])c8[2H])cc7)nc(-c7ccc8ccccc8c7)n6)ccc5c4)c3c2c1]

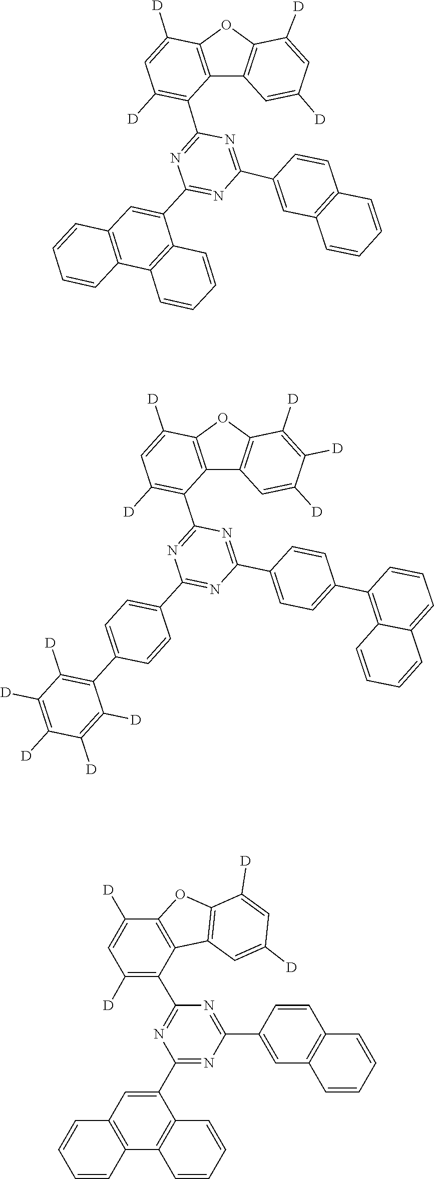 [SMILES: [2H]c1cc([2H])c2oc3c([2H])cc([2H])c(-c4nc(-c5ccc6ccccc6c5)nc(-c5cc6ccccc6c6ccccc56)n4)c3c2c1 ; [2H]c1cc2c(oc3c([2H])cc([2H])c(-c4nc(-c5ccc(-c6c([2H])c([2H])c([2H])c([2H])c6[2H])cc5)nc(-c5ccc(-c6cccc7ccccc67)cc5)n4)c32)c([2H])c1[2H] ; [2H]c1cc([2H])c2oc3c([2H])cc([2H])c(-c4nc(-c5ccc6ccccc6c5)nc(-c5cc6ccccc6c6ccccc56)n4)c3c2c1]

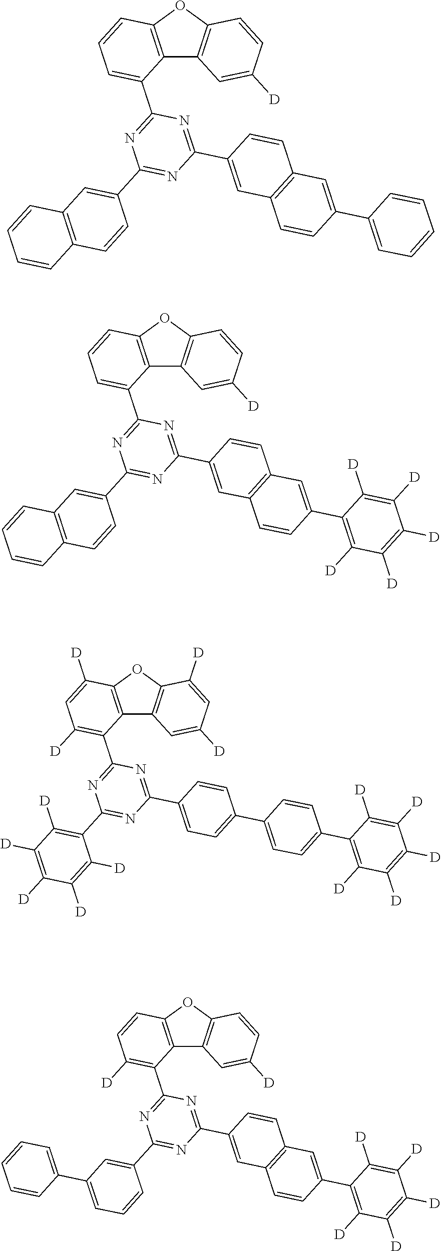 [SMILES: [2H]c1ccc2oc3cccc(-c4nc(-c5ccc6ccccc6c5)nc(-c5ccc6cc(-c7ccccc7)ccc6c5)n4)c3c2c1 ; [2H]c1ccc2oc3cccc(-c4nc(-c5ccc6ccccc6c5)nc(-c5ccc6cc(-c7c([2H])c([2H])c([2H])c([2H])c7[2H])ccc6c5)n4)c3c2c1 ; [2H]c1cc([2H])c2oc3c([2H])cc([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3ccc([2H])c(-c4nc(-c5cccc(-c6ccccc6)c5)nc(-c5ccc6cc(-c7c([2H])c([2H])c([2H])c([2H])c7[2H])ccc6c5)n4)c3c2c1]

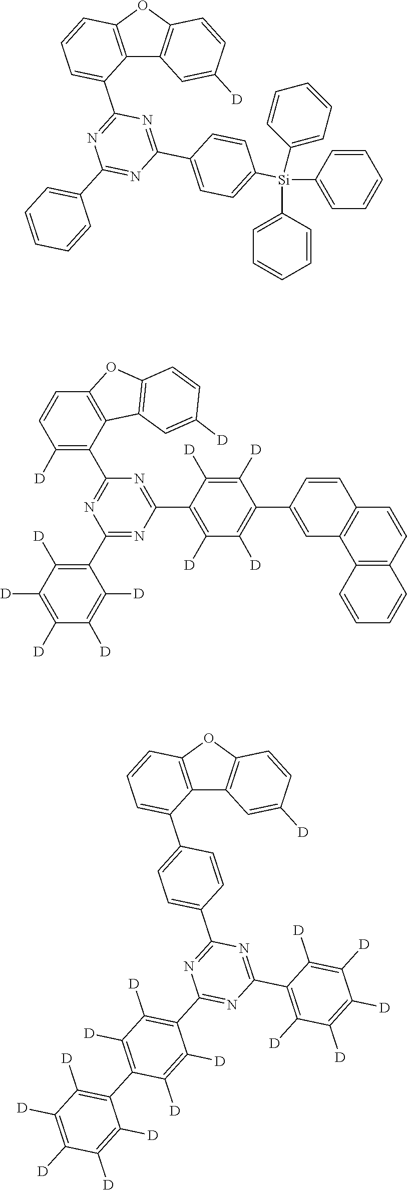 [SMILES: [2H]c1ccc2oc3cccc(-c4nc(-c5ccccc5)nc(-c5ccc([Si](c6ccccc6)(c6ccccc6)c6ccccc6)cc5)n4)c3c2c1 ; [2H]c1ccc2oc3ccc([2H])c(-c4nc(-c5c([2H])c([2H])c([2H])c([2H])c5[2H])nc(-c5c([2H])c([2H])c(-c6ccc7ccc8ccccc8c7c6)c([2H])c5[2H])n4)c3c2c1 ; [2H]c1ccc2oc3cccc(-c4ccc(-c5nc(-c6c([2H])c([2H])c([2H])c([2H])c6[2H])nc(-c6c([2H])c([2H])c(-c7c([2H])c([2H])c([2H])c([2H])c7[2H])c([2H])c6[2H])n5)cc4)c3c2c1]

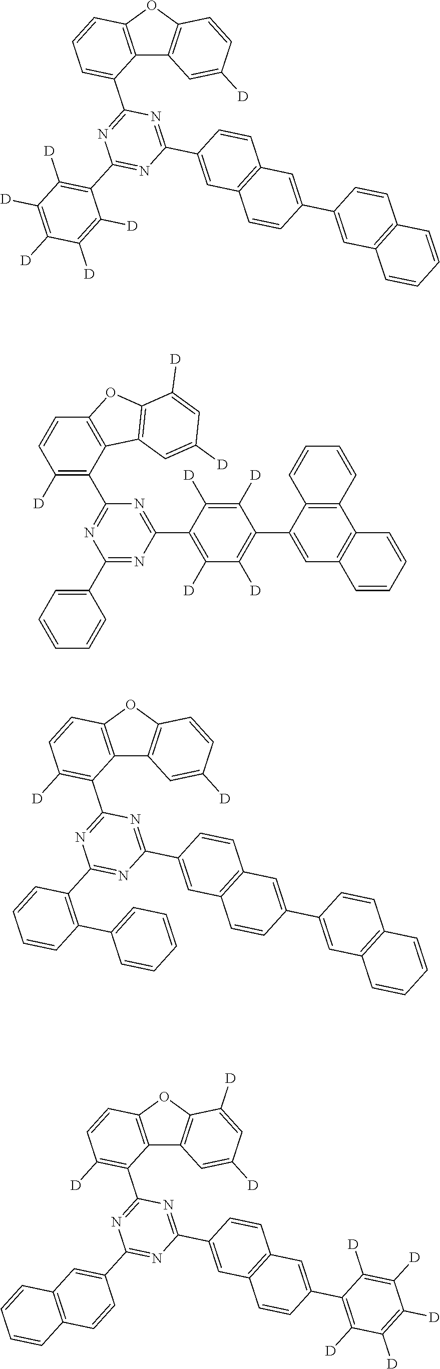 [SMILES: [2H]c1ccc2oc3cccc(-c4nc(-c5ccc6cc(-c7ccc8ccccc8c7)ccc6c5)nc(-c5c([2H])c([2H])c([2H])c([2H])c5[2H])n4)c3c2c1 ; [2H]c1cc([2H])c2oc3ccc([2H])c(-c4nc(-c5ccccc5)nc(-c5c([2H])c([2H])c(-c6cc7ccccc7c7ccccc67)c([2H])c5[2H])n4)c3c2c1 ; [2H]c1ccc2oc3ccc([2H])c(-c4nc(-c5ccc6cc(-c7ccc8ccccc8c7)ccc6c5)nc(-c5ccccc5-c5ccccc5)n4)c3c2c1 ; [2H]c1cc([2H])c2oc3ccc([2H])c(-c4nc(-c5ccc6ccccc6c5)nc(-c5ccc6cc(-c7c([2H])c([2H])c([2H])c([2H])c7[2H])ccc6c5)n4)c3c2c1]

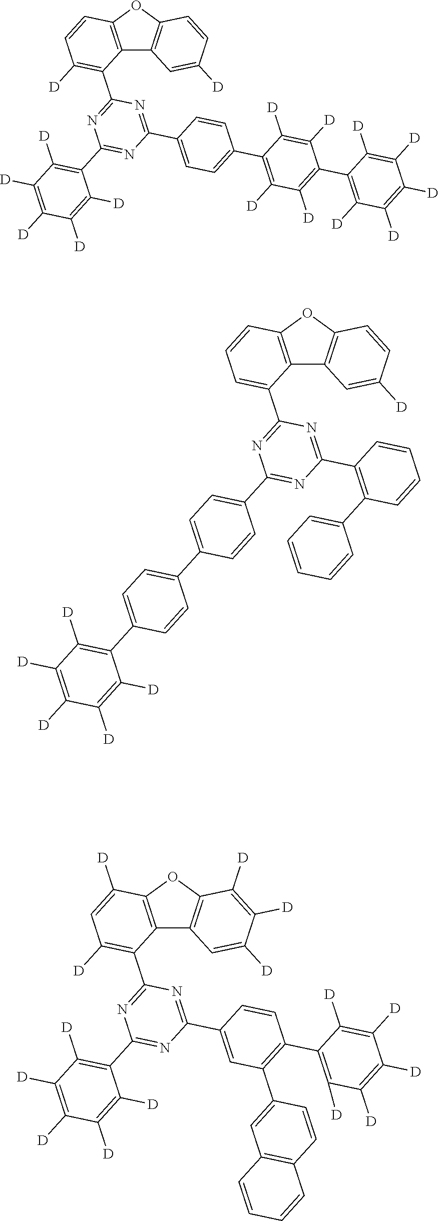 [SMILES: [2H]c1ccc2oc3ccc([2H])c(-c4nc(-c5ccc(-c6c([2H])c([2H])c(-c7c([2H])c([2H])c([2H])c([2H])c7[2H])c([2H])c6[2H])cc5)nc(-c5c([2H])c([2H])c([2H])c([2H])c5[2H])n4)c3c2c1 ; [2H]c1ccc2oc3cccc(-c4nc(-c5ccc(-c6ccc(-c7c([2H])c([2H])c([2H])c([2H])c7[2H])cc6)cc5)nc(-c5ccccc5-c5ccccc5)n4)c3c2c1 ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5c([2H])c([2H])c([2H])c([2H])c5[2H])n4)c32)c([2H])c1[2H]]

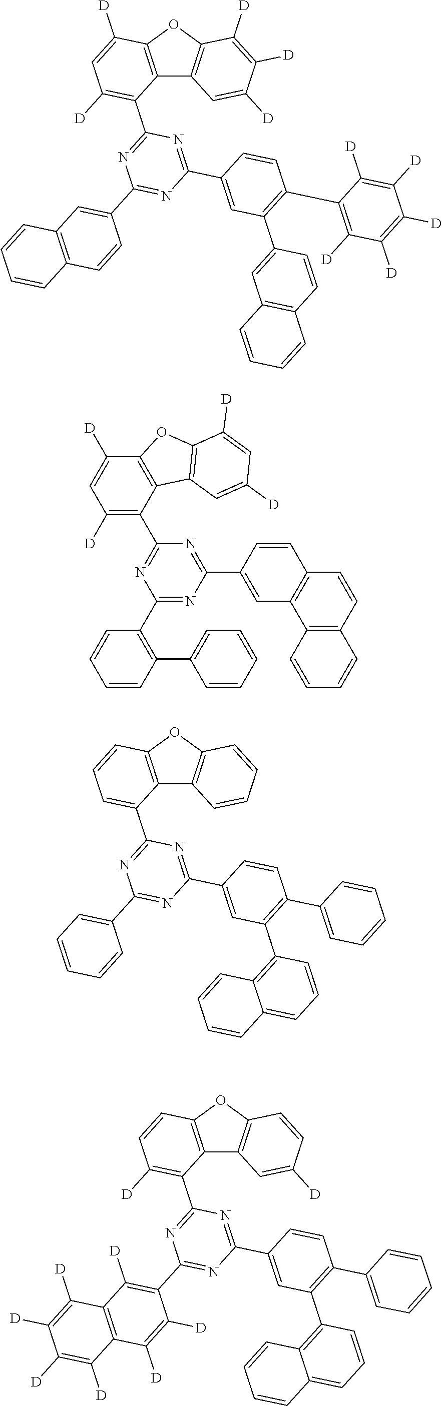

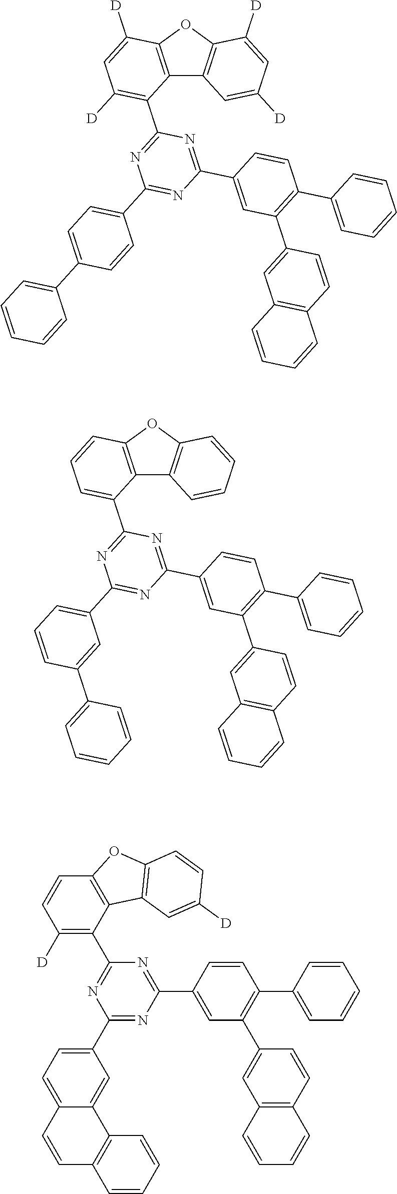 [SMILES: [2H]c1cc([2H])c2oc3c([2H])cc([2H])c(-c4nc(-c5ccc(-c6ccccc6)cc5)nc(-c5ccc(-c6ccccc6)c(-c6ccc7ccccc7c6)c5)n4)c3c2c1 ; c1ccc(-c2cccc(-c3nc(-c4ccc(-c5ccccc5)c(-c5ccc6ccccc6c5)c4)nc(-c4cccc5oc6ccccc6c45)n3)c2)cc1 ; [2H]c1ccc2oc3ccc([2H])c(-c4nc(-c5ccc(-c6ccccc6)c(-c6ccc7ccccc7c6)c5)nc(-c5ccc6ccc7ccccc7c6c5)n4)c3c2c1]

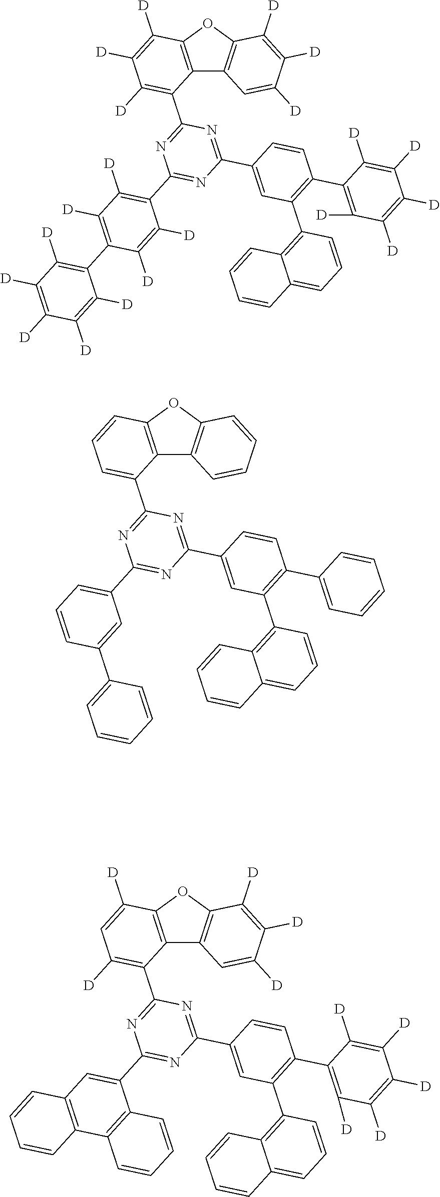 [SMILES: [2H]c1cc2c(oc3c([2H])c([2H])c([2H])c(-c4nc(-c5ccc(-c6c([2H])c([2H])c([2H])c([2H])c6[2H])c(-c6cccc7ccccc67)c5)nc(-c5c([2H])c([2H])c(-c6c([2H])c([2H])c([2H])c([2H])c6[2H])c([2H])c5[2H])n4)c32)c([2H])c1[2H] ; c1ccc(-c2cccc(-c3nc(-c4ccc(-c5ccccc5)c(-c5cccc6ccccc56)c4)nc(-c4cccc5oc6ccccc6c45)n3)c2)cc1 ; [2H]c1cc2c(oc3c([2H])cc([2H])c(-c4nc(-c5ccc(-c6c([2H])c([2H])c([2H])c([2H])c6[2H])c(-c6cccc7ccccc67)c5)nc(-c5cc6ccccc6c6ccccc56)n4)c32)c([2H])c1[2H]]

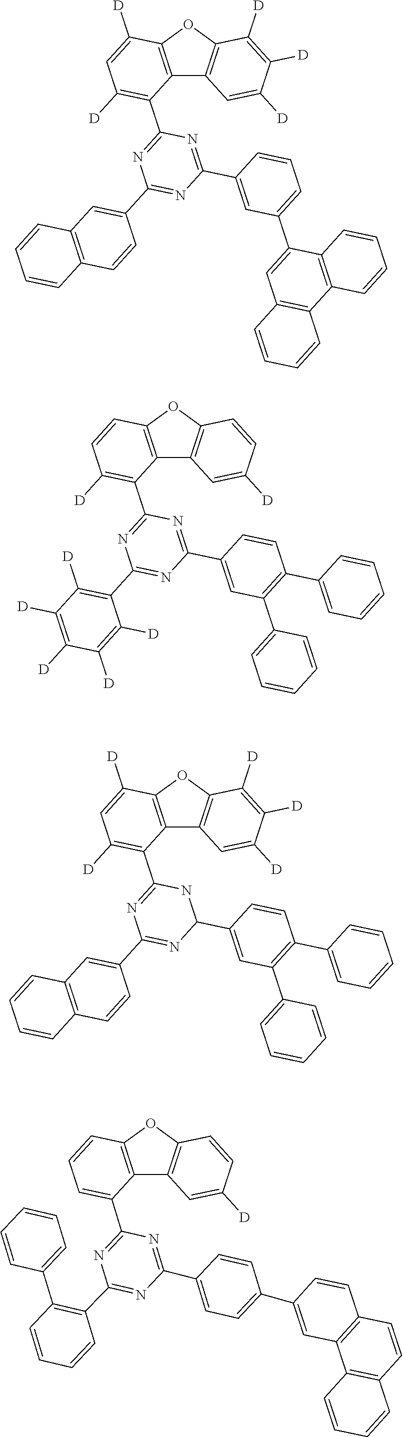

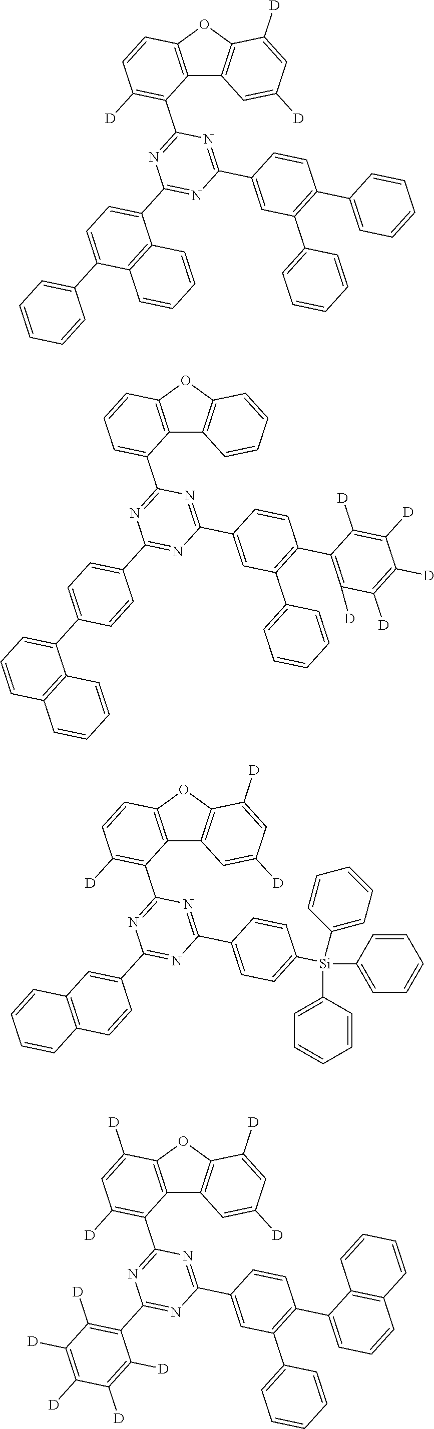 [SMILES: [2H]c1cc([2H])c2oc3ccc([2H])c(-c4nc(-c5ccc(-c6ccccc6)c(-c6ccccc6)c5)nc(-c5ccc(-c6ccccc6)c6ccccc56)n4)c3c2c1 ; [2H]c1c([2H])c([2H])c(-c2ccc(-c3nc(-c4ccc(-c5cccc6ccccc56)cc4)nc(-c4cccc5oc6ccccc6c45)n3)cc2-c2ccccc2)c([2H])c1[2H] ; [2H]c1cc([2H])c2oc3ccc([2H])c(-c4nc(-c5ccc([Si](c6ccccc6)(c6ccccc6)c6ccccc6)cc5)nc(-c5ccc6ccccc6c5)n4)c3c2c1 ; [2H]c1cc([2H])c2oc3c([2H])cc([2H])c(-c4nc(-c5ccc(-c6cccc7ccccc67)c(-c6ccccc6)c5)nc(-c5c([2H])c([2H])c([2H])c([2H])c5[2H])n4)c3c2c1]

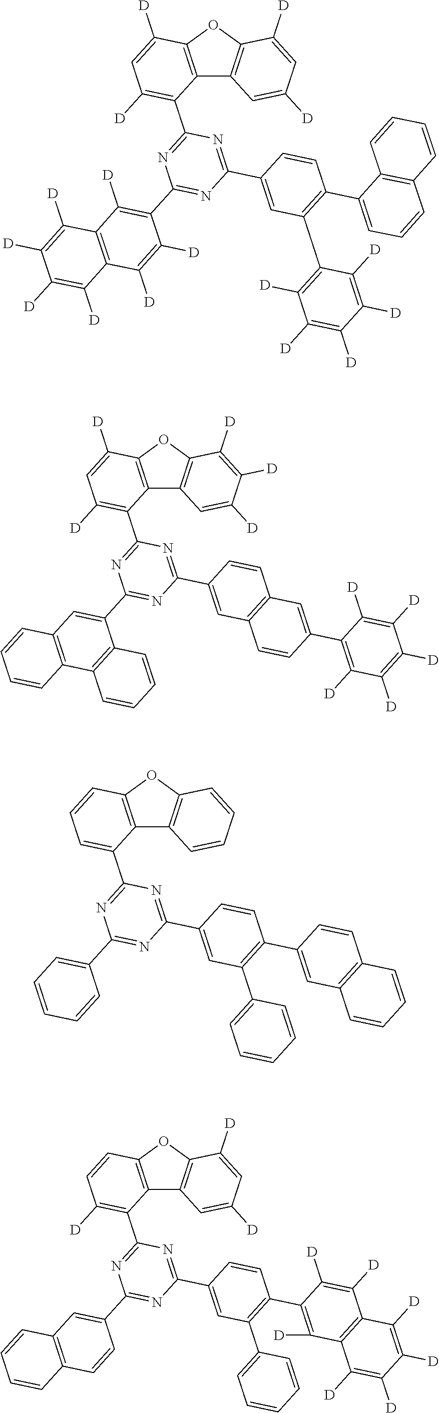

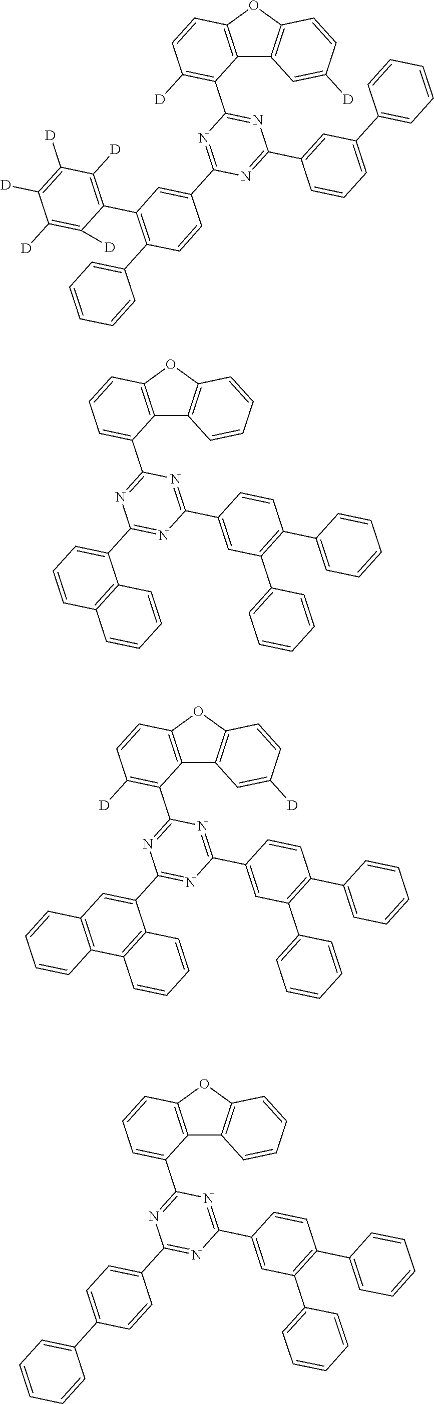 [SMILES: [2H]c1ccc2oc3ccc([2H])c(-c4nc(-c5cccc(-c6ccccc6)c5)nc(-c5ccc(-c6ccccc6)c(-c6c([2H])c([2H])c([2H])c([2H])c6[2H])c5)n4)c3c2c1 ; c1ccc(-c2ccc(-c3nc(-c4cccc5ccccc45)nc(-c4cccc5oc6ccccc6c45)n3)cc2-c2ccccc2)cc1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1 ; c1ccc(-c2ccc(-c3nc(-c4ccc(-c5ccccc5)c(-c5ccccc5)c4)nc(-c4cccc5oc6ccccc6c45)n3)cc2)cc1]

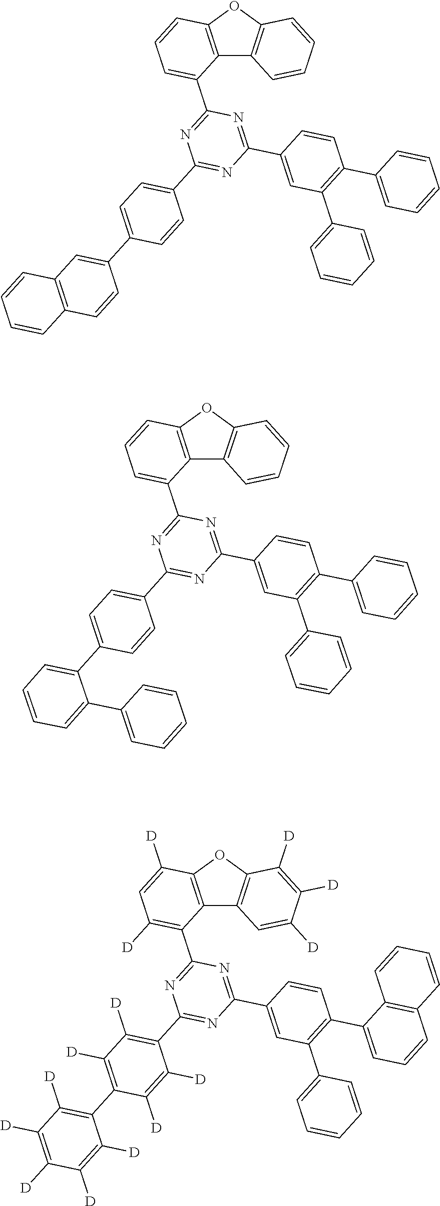 [SMILES: c1ccc(-c2ccc(-c3nc(-c4ccc(-c5ccc6ccccc6c5)cc4)nc(-c4cccc5oc6ccccc6c45)n3)cc2-c2ccccc2)cc1 ; c1ccc(-c2ccccc2-c2ccc(-c3nc(-c4ccc(-c5ccccc5)c(-c5ccccc5)c4)nc(-c4cccc5oc6ccccc6c45)n3)cc2)cc1 ; [2H]c1cc2c(oc3c([2H])cc([2H])c(-c4nc(-c5ccc(-c6cccc7ccccc67)c(-c6ccccc6)c5)nc(-c5c([2H])c([2H])c(-c6c([2H])c([2H])c([2H])c([2H])c6[2H])c([2H])c5[2H])n4)c32)c([2H])c1[2H]]

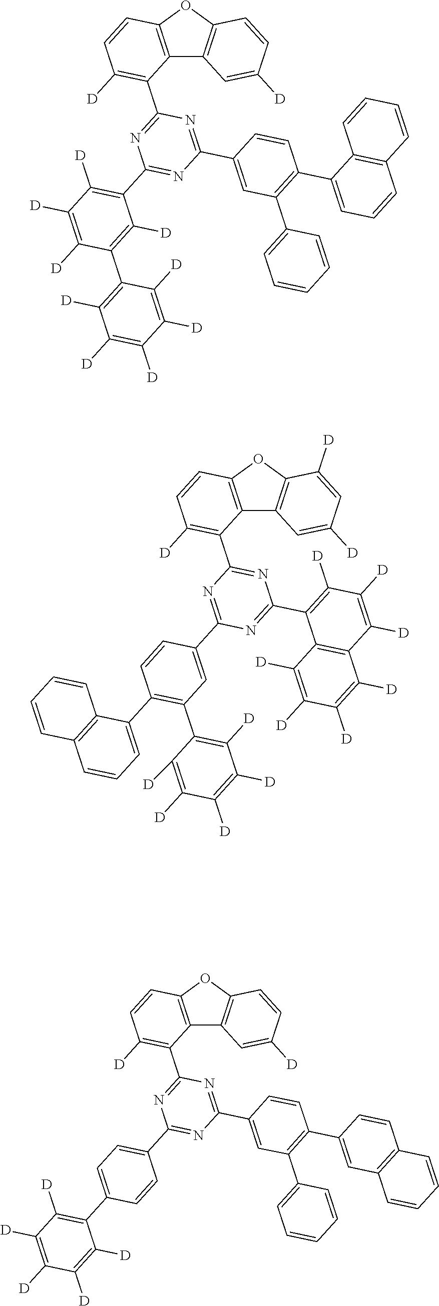 [SMILES: [2H]c1ccc2oc3ccc([2H])c(-c4nc(-c5ccc(-c6cccc7ccccc67)c(-c6ccccc6)c5)nc(-c5c([2H])c([2H])c([2H])c(-c6c([2H])c([2H])c([2H])c([2H])c6[2H])c5[2H])n4)c3c2c1 ; [2H]c1cc([2H])c2oc3ccc([2H])c(-c4nc(-c5ccc(-c6cccc7ccccc67)c(-c6c([2H])c([2H])c([2H])c([2H])c6[2H])c5)nc(-c5c([2H])c([2H])c([2H])c6c([2H])c([2H])c([2H])c([2H])c56)n4)c3c2c1 ; [2H]c1ccc2oc3ccc([2H])c(-c4nc(-c5ccc(-c6c([2H])c([2H])c([2H])c([2H])c6[2H])cc5)nc(-c5ccc(-c6ccc7ccccc7c6)c(-c6ccccc6)c5)n4)c3c2c1]

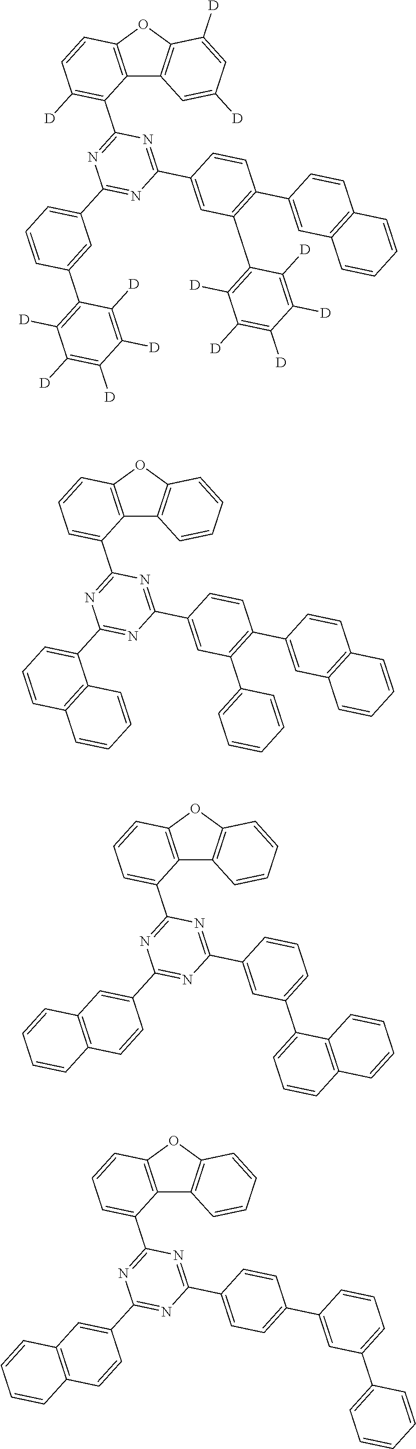 [SMILES: [2H]c1cc([2H])c2oc3ccc([2H])c(-c4nc(-c5cccc(-c6c([2H])c([2H])c([2H])c([2H])c6[2H])c5)nc(-c5ccc(-c6ccc7ccccc7c6)c(-c6c([2H])c([2H])c([2H])c([2H])c6[2H])c5)n4)c3c2c1 ; c1ccc(-c2cc(-c3nc(-c4cccc5ccccc45)nc(-c4cccc5oc6ccccc6c45)n3)ccc2-c2ccc3ccccc3c2)cc1 ; c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1]

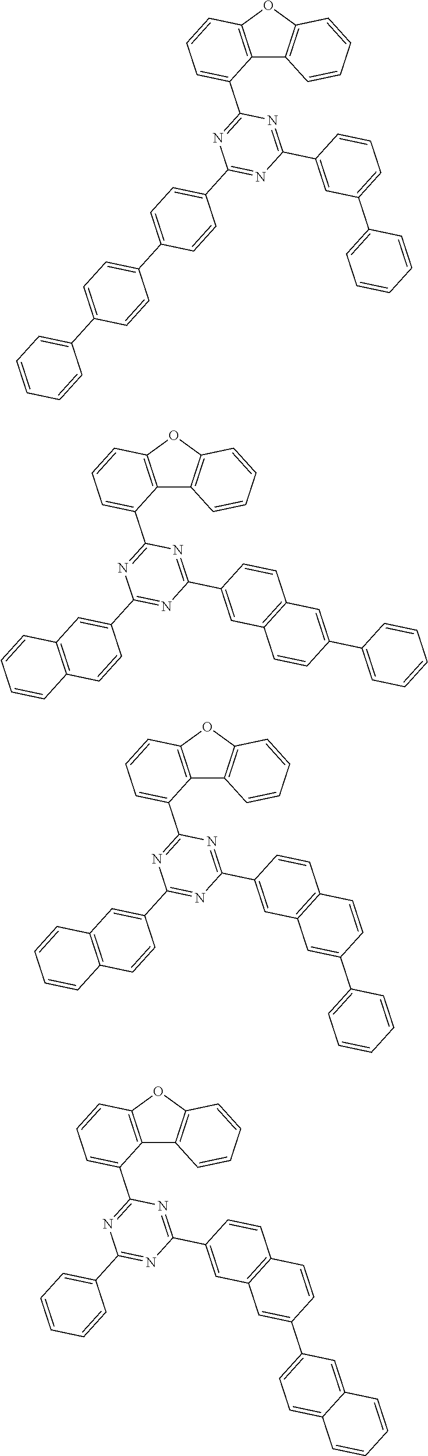 [SMILES: c1ccc(-c2ccc(-c3ccc(-c4nc(-c5cccc(-c6ccccc6)c5)nc(-c5cccc6oc7ccccc7c56)n4)cc3)cc2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1]

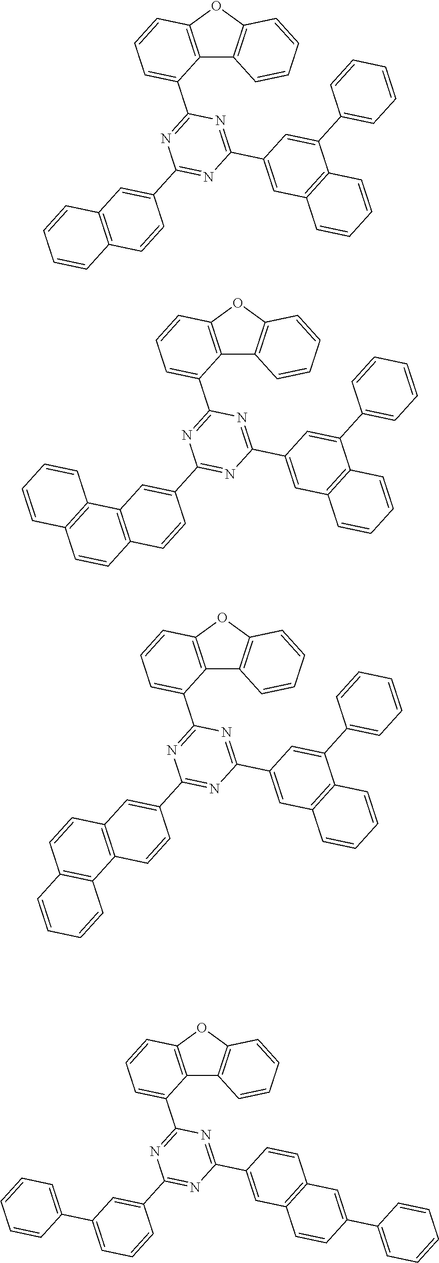 [SMILES: c1ccc(-c2cc(-c3nc(-c4ccc5ccccc5c4)nc(-c4cccc5oc6ccccc6c45)n3)cc3ccccc23)cc1 ; c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1 ; c1ccc(-c2cccc(-c3nc(-c4ccc5cc(-c6ccccc6)ccc5c4)nc(-c4cccc5oc6ccccc6c45)n3)c2)cc1]

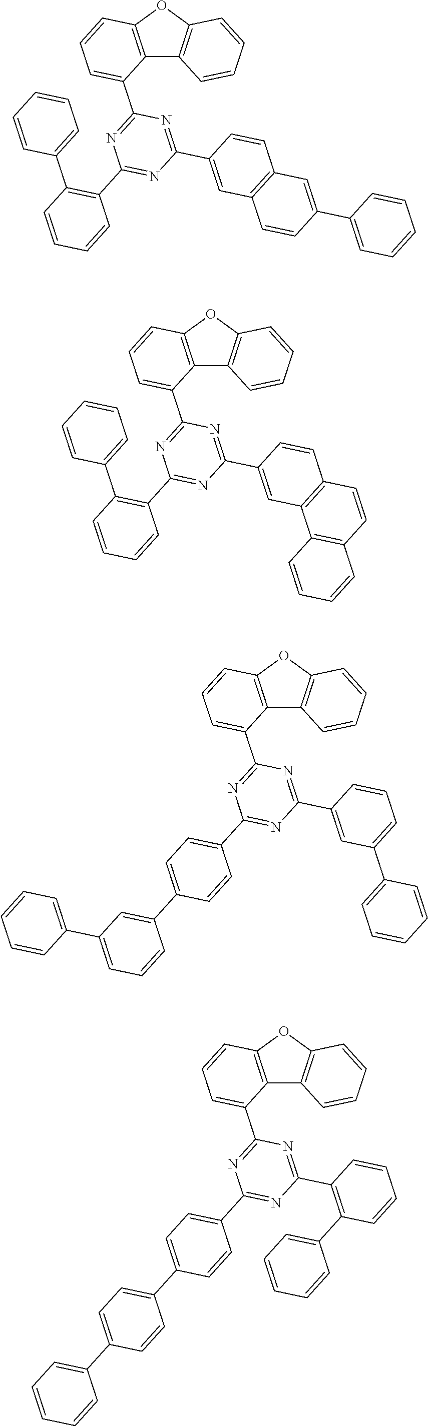 [SMILES: c1ccc(-c2ccc3cc(-c4nc(-c5ccccc5-c5ccccc5)nc(-c5cccc6oc7ccccc7c56)n4)ccc3c2)cc1 ; c1ccc(-c2ccccc2-c2nc(-c3ccc4ccc5ccccc5c4c3)nc(-c3cccc4oc5ccccc5c34)n2)cc1 ; c1ccc(-c2cccc(-c3ccc(-c4nc(-c5cccc(-c6ccccc6)c5)nc(-c5cccc6oc7ccccc7c56)n4)cc3)c2)cc1 ; c1ccc(-c2ccc(-c3ccc(-c4nc(-c5ccccc5-c5ccccc5)nc(-c5cccc6oc7ccccc7c56)n4)cc3)cc2)cc1]

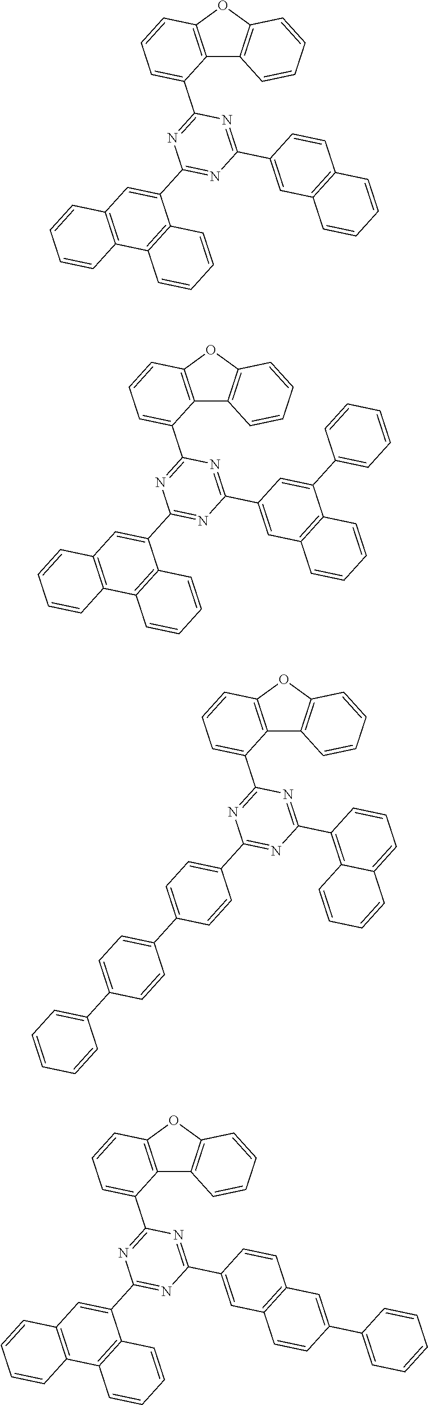 [SMILES: c1ccc2cc(-c3nc(-c4cc5ccccc5c5ccccc45)nc(-c4cccc5oc6ccccc6c45)n3)ccc2c1 ; c1ccc(-c2cc(-c3nc(-c4cc5ccccc5c5ccccc45)nc(-c4cccc5oc6ccccc6c45)n3)cc3ccccc23)cc1 ; c1ccc(-c2ccc(-c3ccc(-c4nc(-c5cccc6ccccc56)nc(-c5cccc6oc7ccccc7c56)n4)cc3)cc2)cc1 ; c1ccc(-c2ccc3cc(-c4nc(-c5cc6ccccc6c6ccccc56)nc(-c5cccc6oc7ccccc7c56)n4)ccc3c2)cc1]

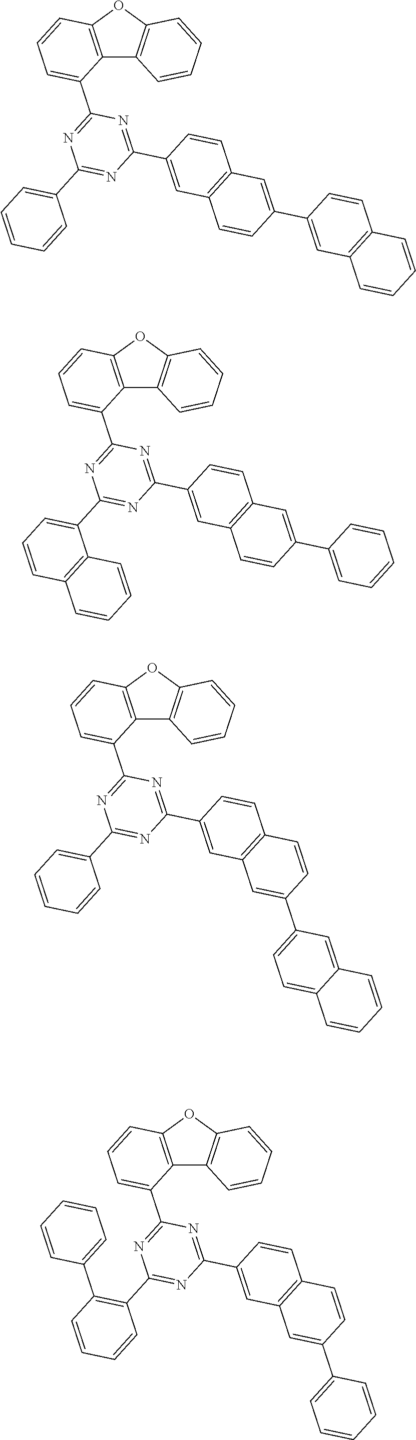 [SMILES: c1ccc(-c2nc(-c3ccc4cc(-c5ccc6ccccc6c5)ccc4c3)nc(-c3cccc4oc5ccccc5c34)n2)cc1 ; c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3ccc(-c4nc(-c5ccccc5-c5ccccc5)nc(-c5cccc6oc7ccccc7c56)n4)cc3c2)cc1]

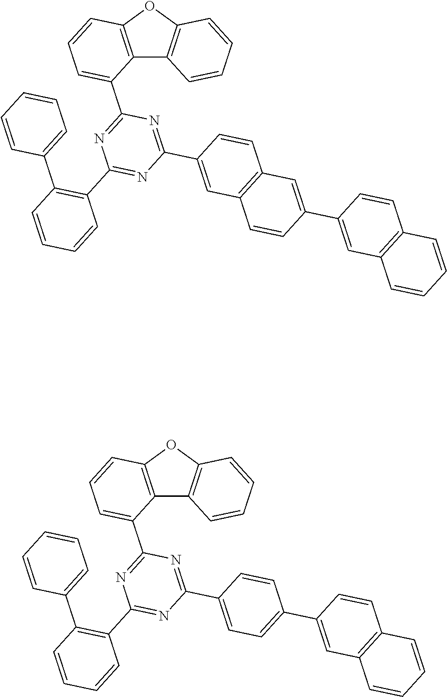 [SMILES: c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1]

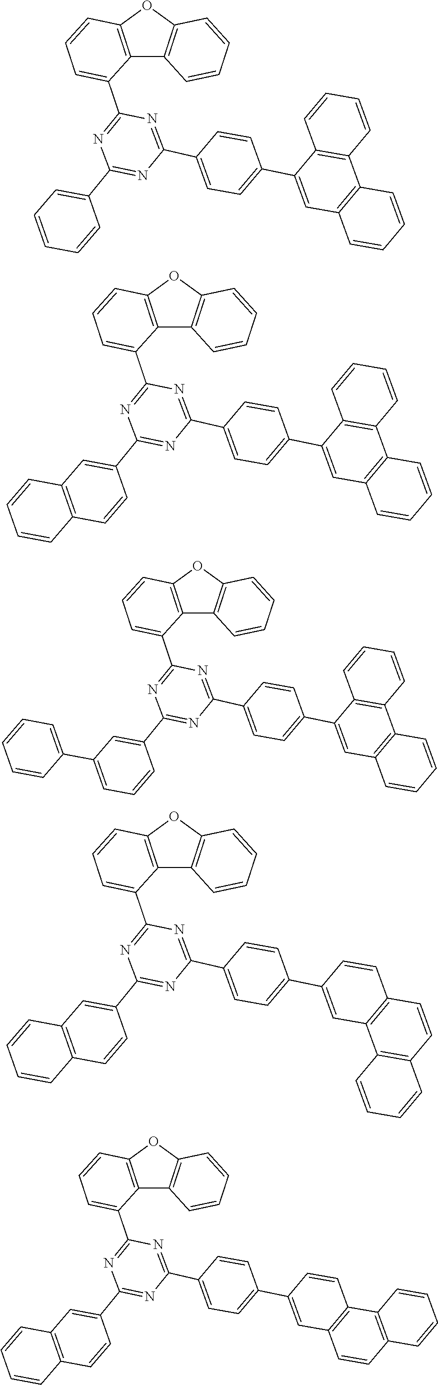

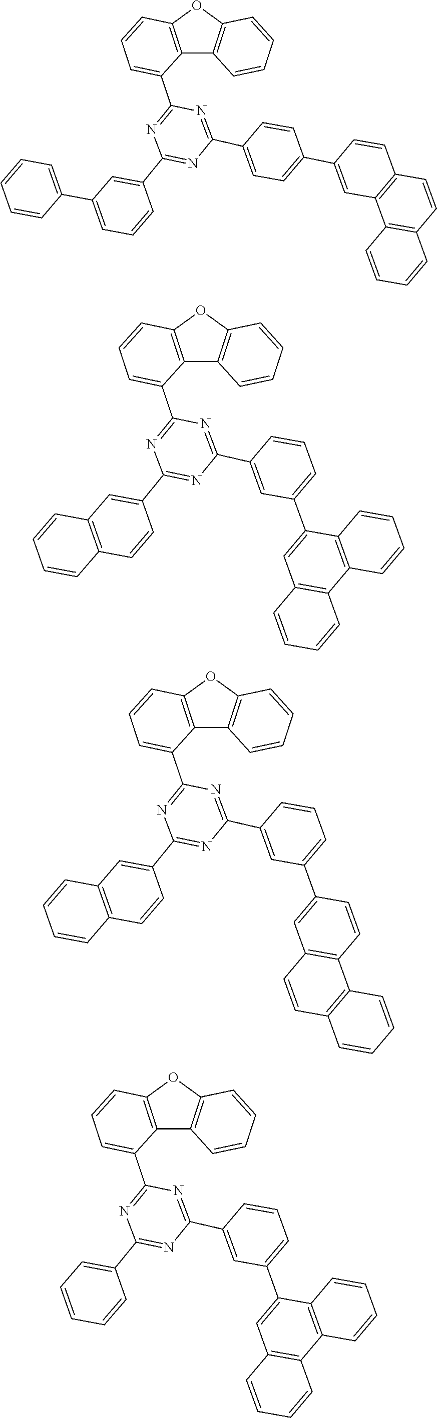 [SMILES: c1ccc(-c2cccc(-c3nc(-c4ccc(-c5ccc6ccc7ccccc7c6c5)cc4)nc(-c4cccc5oc6ccccc6c45)n3)c2)cc1 ; c1cc(-c2nc(-c3ccc4ccccc4c3)nc(-c3cccc4oc5ccccc5c34)n2)cc(-c2cc3ccccc3c3ccccc23)c1 ; c1cc(-c2ccc3c(ccc4ccccc43)c2)cc(-c2nc(-c3ccc4ccccc4c3)nc(-c3cccc4oc5ccccc5c34)n2)c1 ; c1ccc(-c2nc(-c3cccc(-c4cc5ccccc5c5ccccc45)c3)nc(-c3cccc4oc5ccccc5c34)n2)cc1]

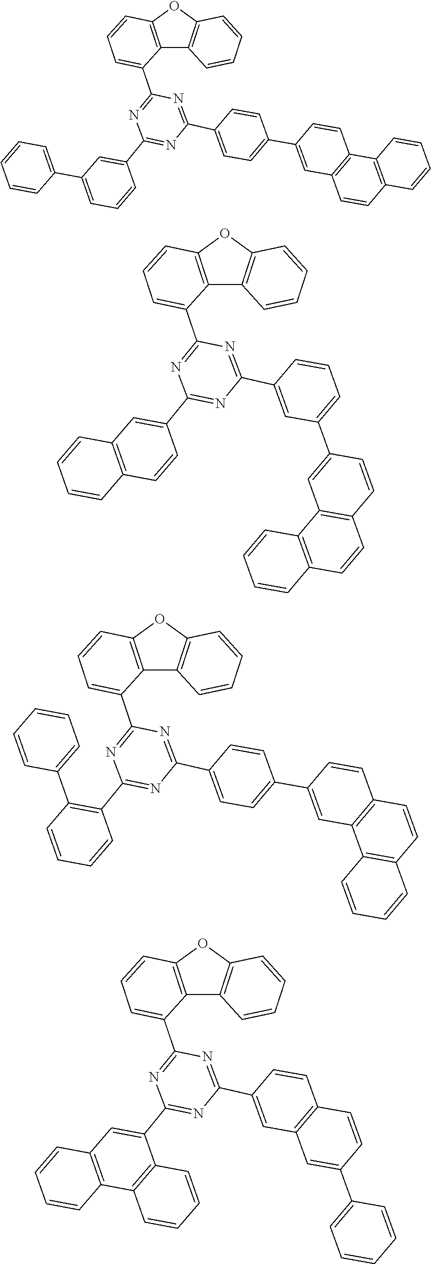 [SMILES: c1ccc(-c2cccc(-c3nc(-c4ccc(-c5ccc6c(ccc7ccccc76)c5)cc4)nc(-c4cccc5oc6ccccc6c45)n3)c2)cc1 ; c1cc(-c2ccc3ccc4ccccc4c3c2)cc(-c2nc(-c3ccc4ccccc4c3)nc(-c3cccc4oc5ccccc5c34)n2)c1 ; c1ccc(-c2ccccc2-c2nc(-c3ccc(-c4ccc5ccc6ccccc6c5c4)cc3)nc(-c3cccc4oc5ccccc5c34)n2)cc1 ; c1ccc(-c2ccc3ccc(-c4nc(-c5cc6ccccc6c6ccccc56)nc(-c5cccc6oc7ccccc7c56)n4)cc3c2)cc1]

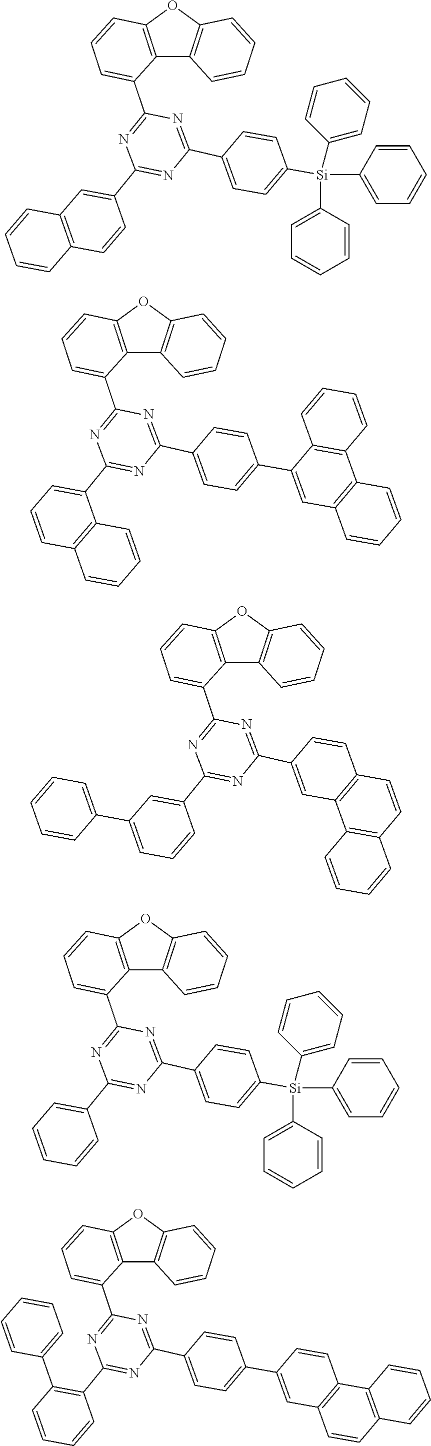 [SMILES: c1ccc([Si](c2ccccc2)(c2ccccc2)c2ccc(-c3nc(-c4ccc5ccccc5c4)nc(-c4cccc5oc6ccccc6c45)n3)cc2)cc1 ; c1ccc2c(-c3nc(-c4ccc(-c5cc6ccccc6c6ccccc56)cc4)nc(-c4cccc5oc6ccccc6c45)n3)cccc2c1 ; c1ccc(-c2cccc(-c3nc(-c4ccc5ccc6ccccc6c5c4)nc(-c4cccc5oc6ccccc6c45)n3)c2)cc1 ; c1ccc(-c2nc(-c3ccc([Si](c4ccccc4)(c4ccccc4)c4ccccc4)cc3)nc(-c3cccc4oc5ccccc5c34)n2)cc1 ; c1ccc(-c2ccccc2-c2nc(-c3ccc(-c4ccc5c(ccc6ccccc65)c4)cc3)nc(-c3cccc4oc5ccccc5c34)n2)cc1]

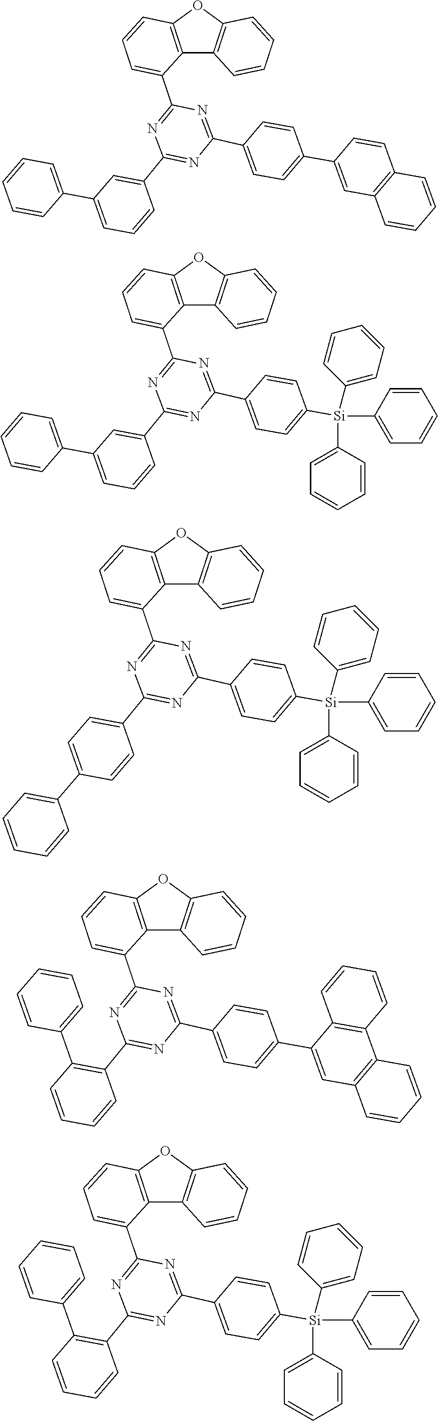

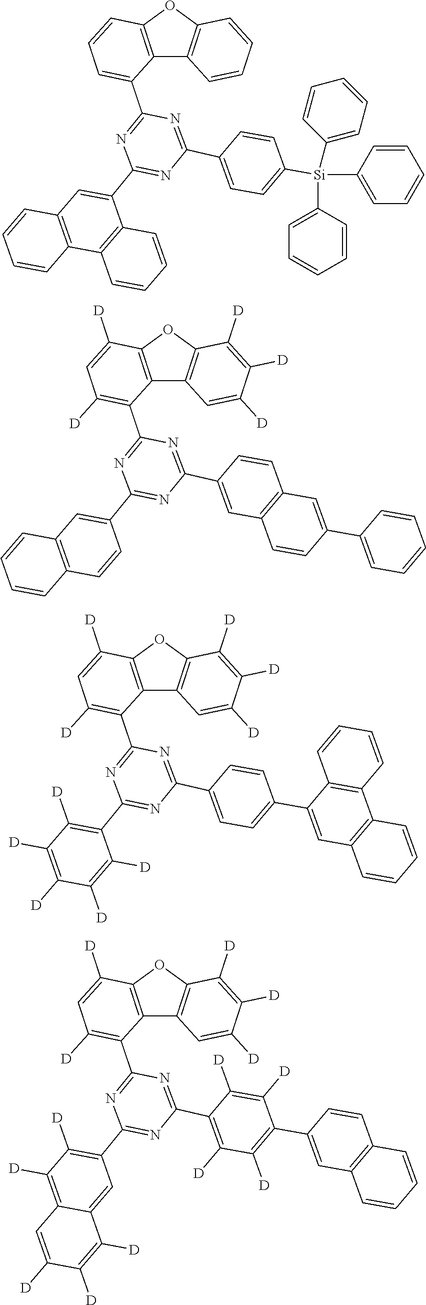

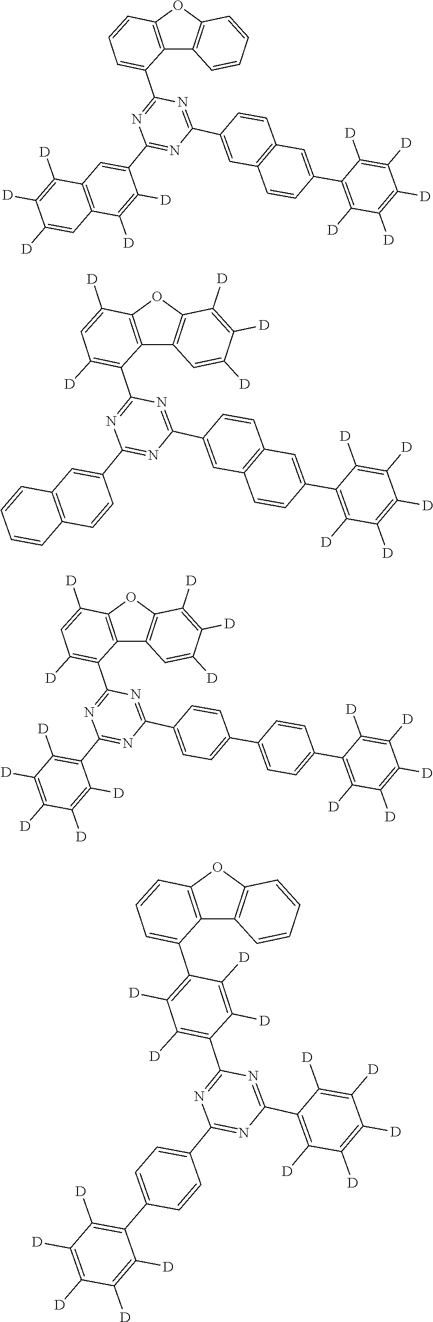

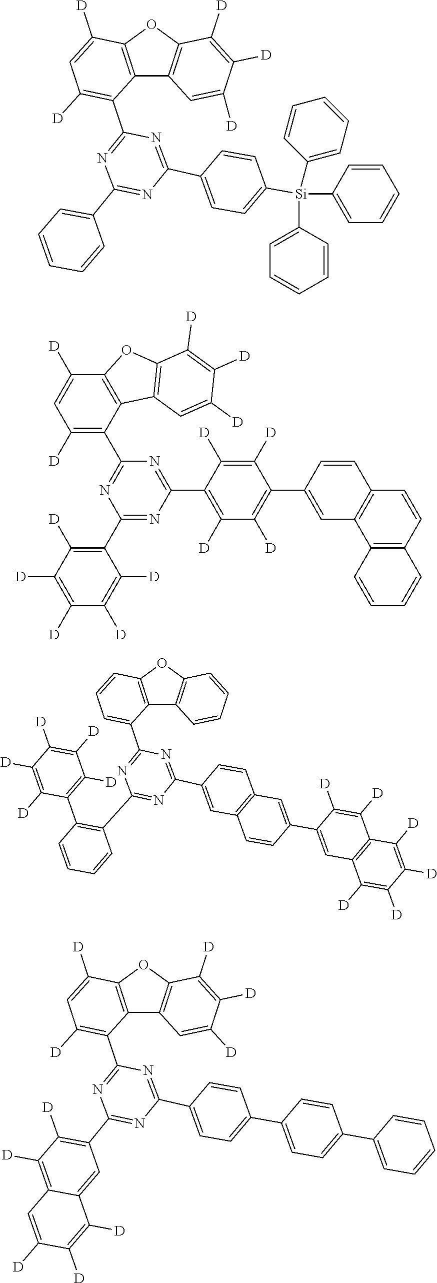

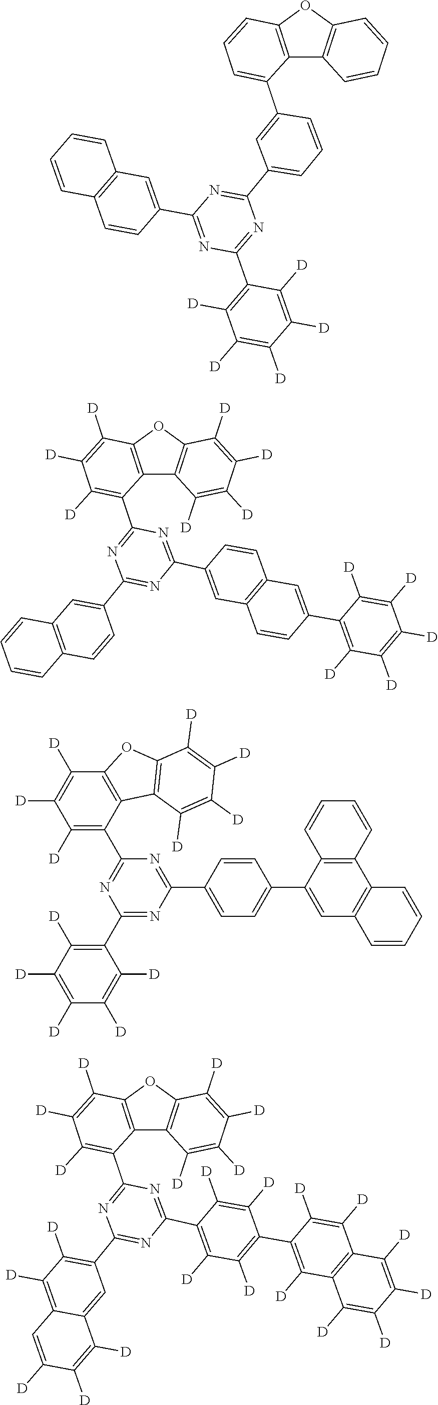

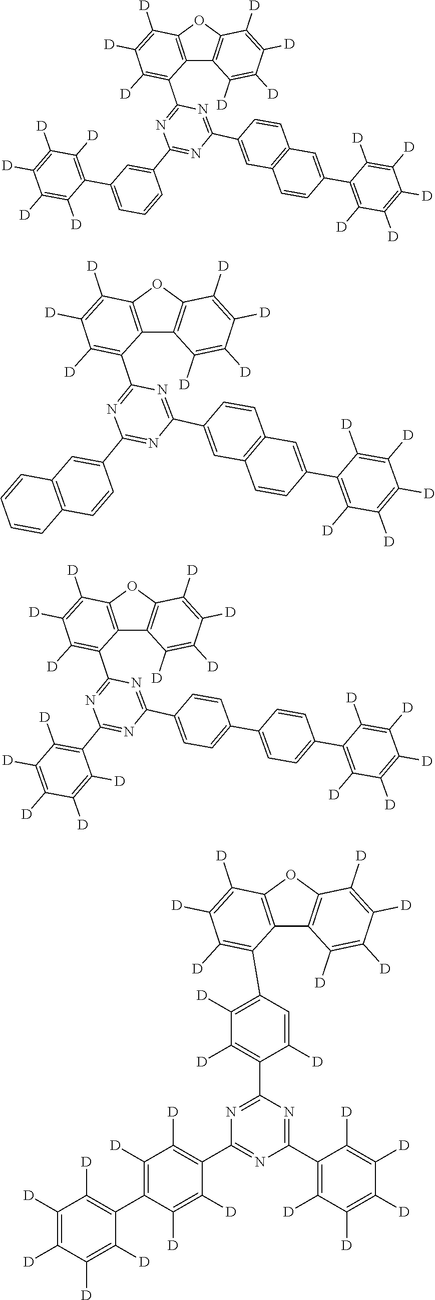

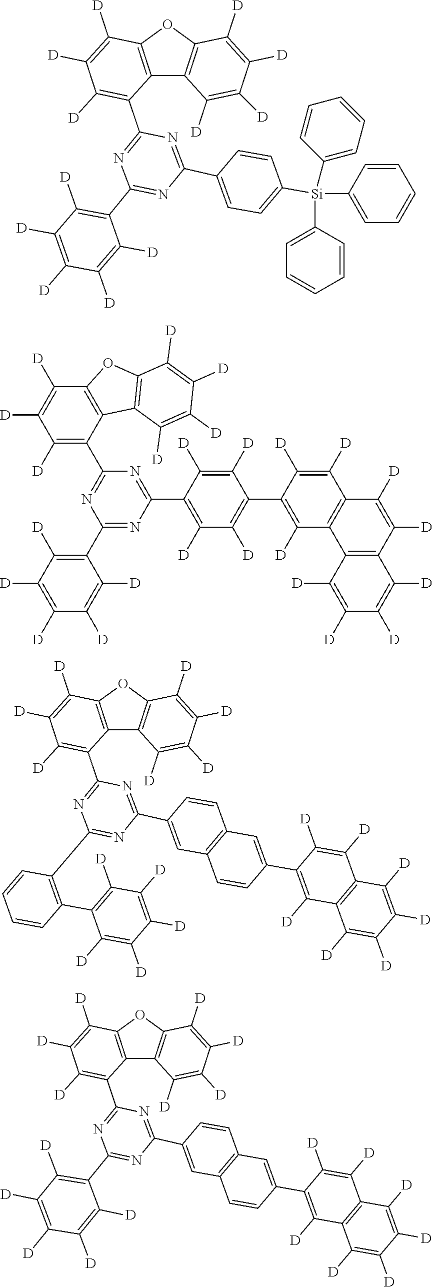

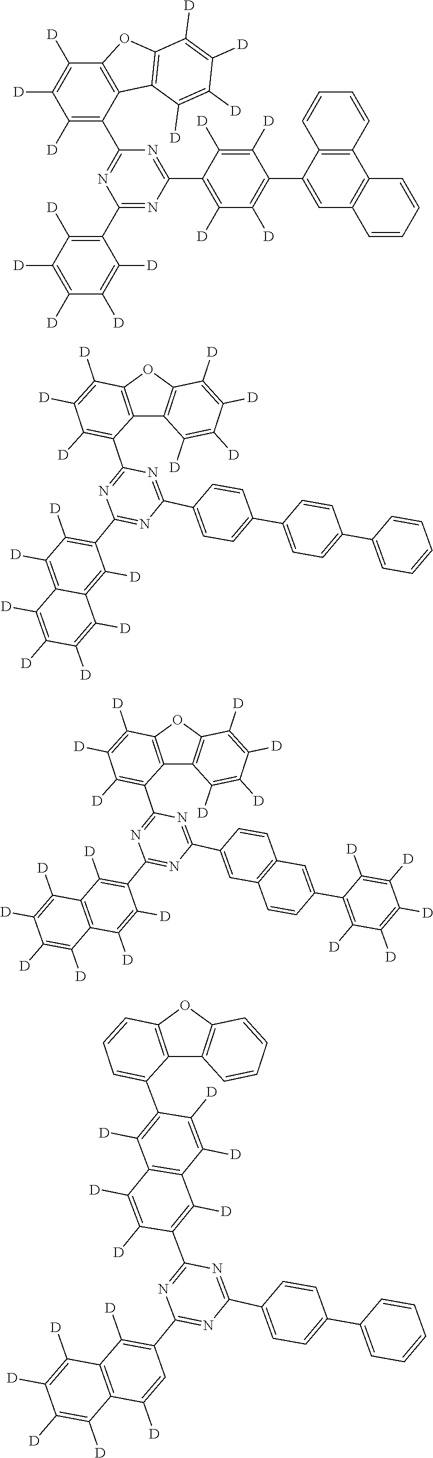

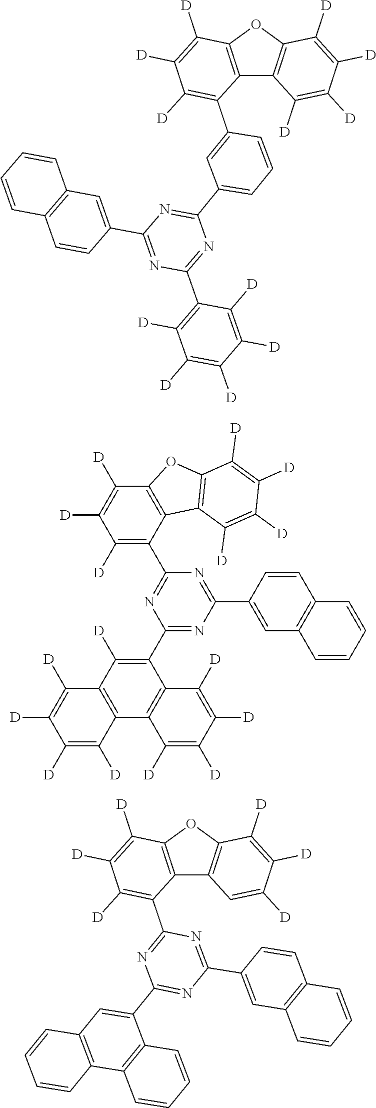

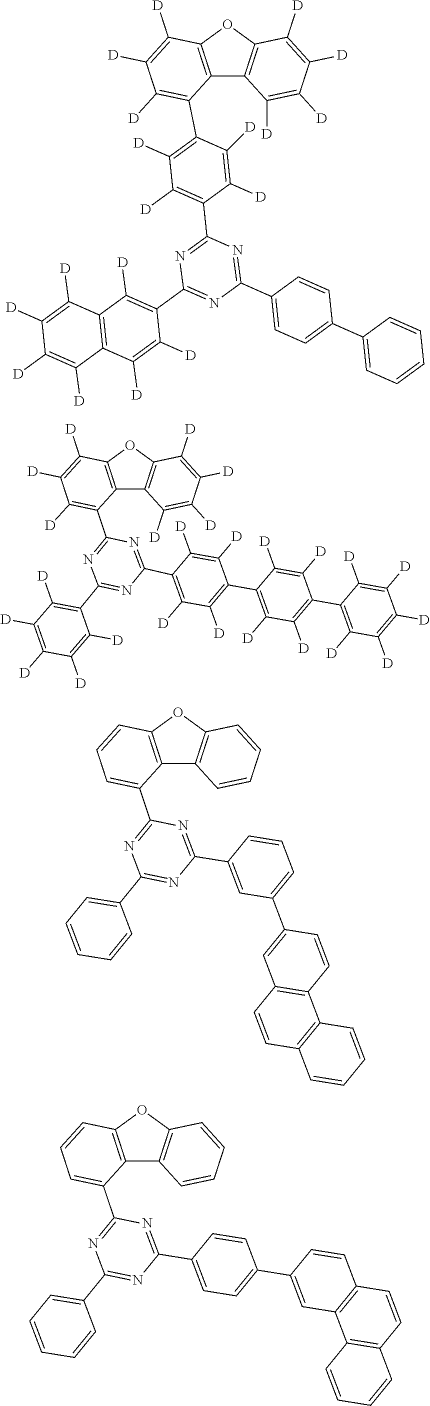

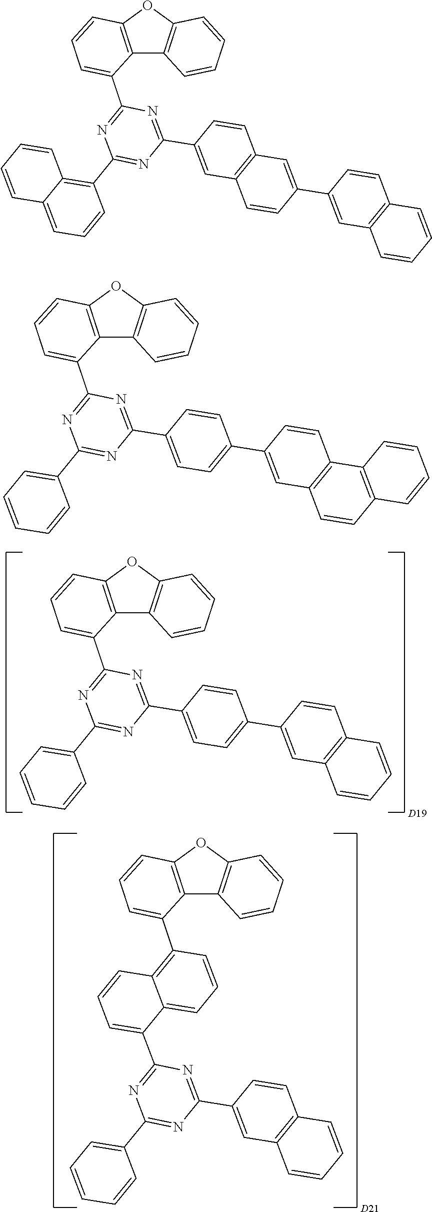

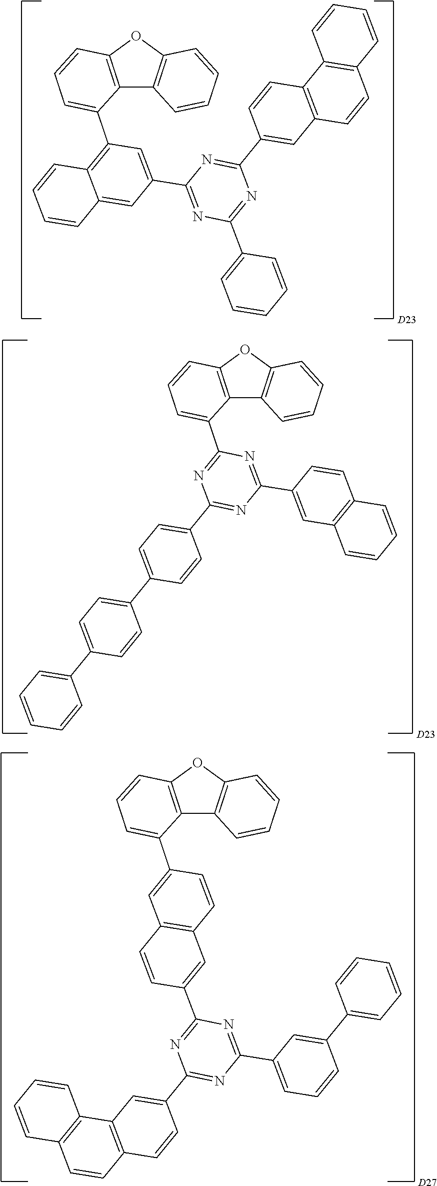

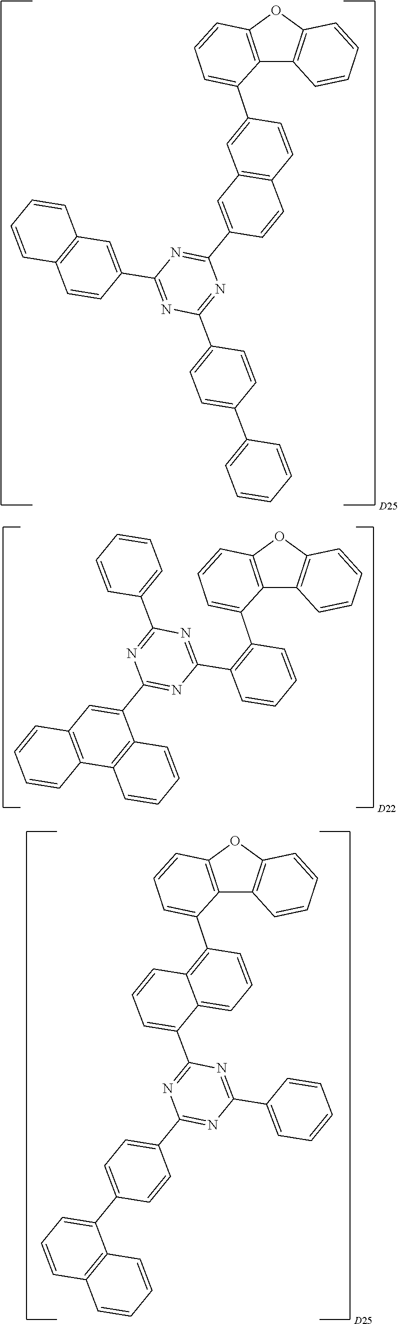

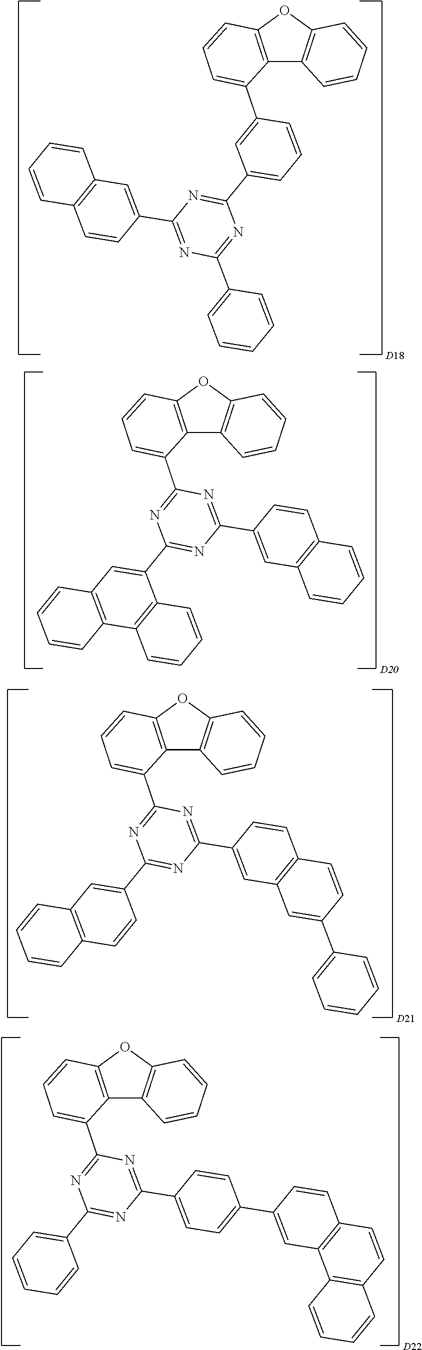

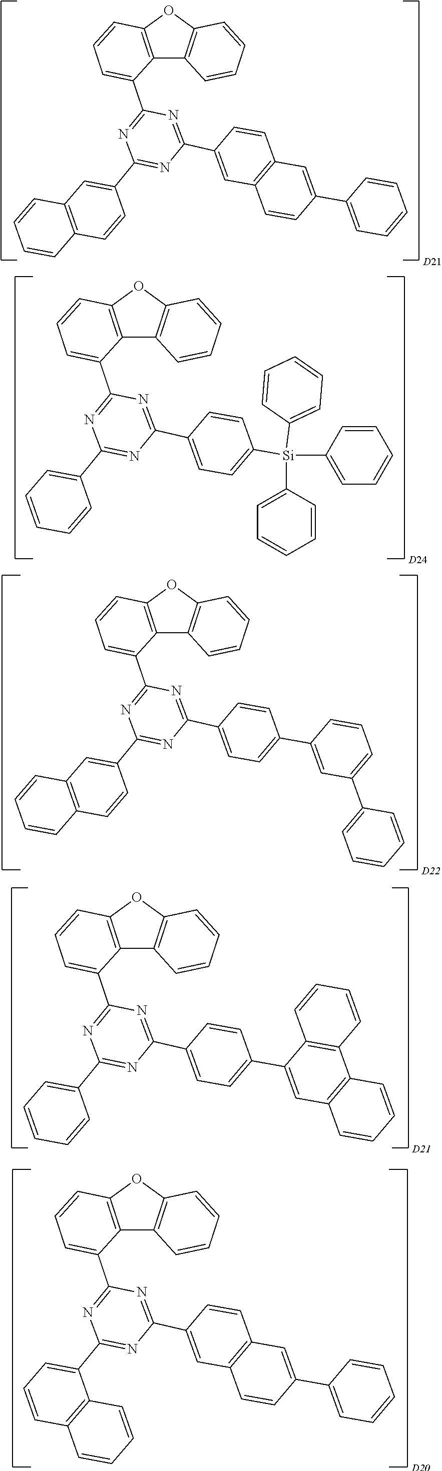

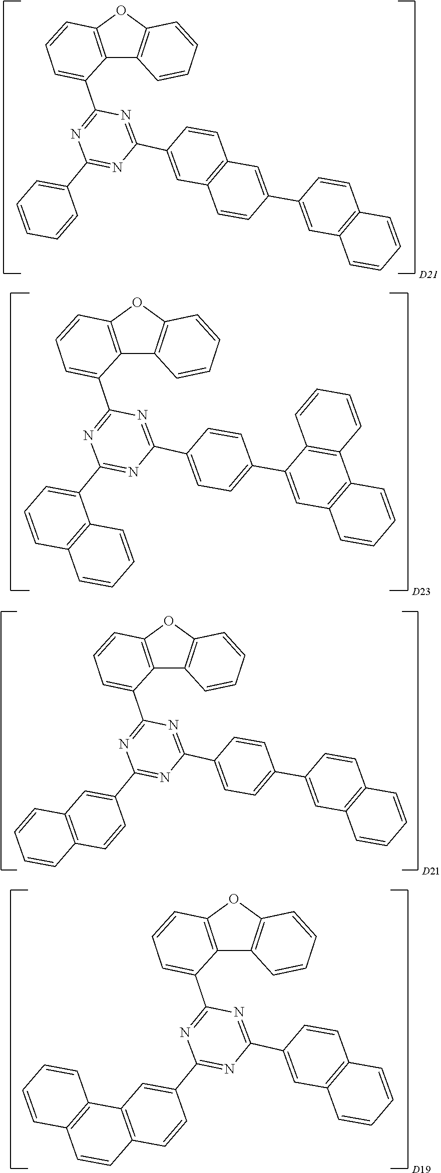

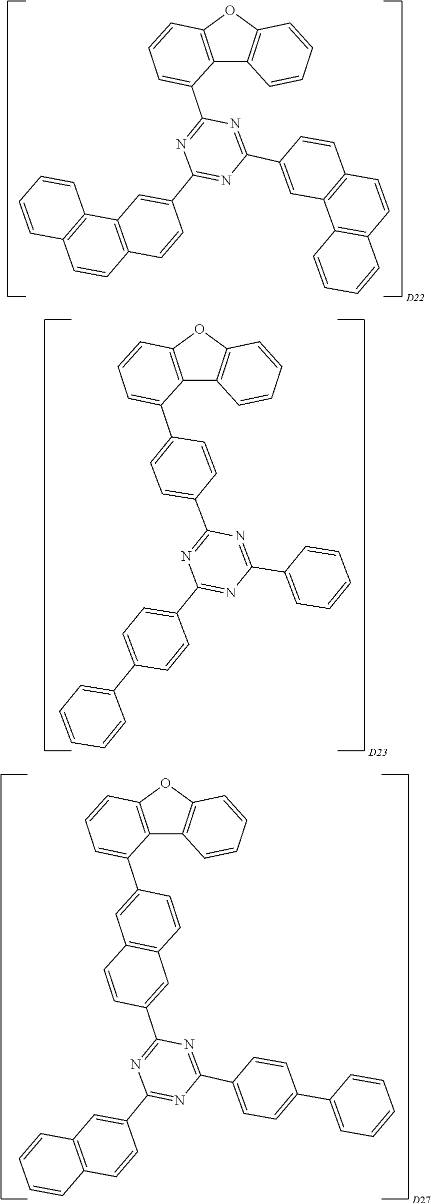

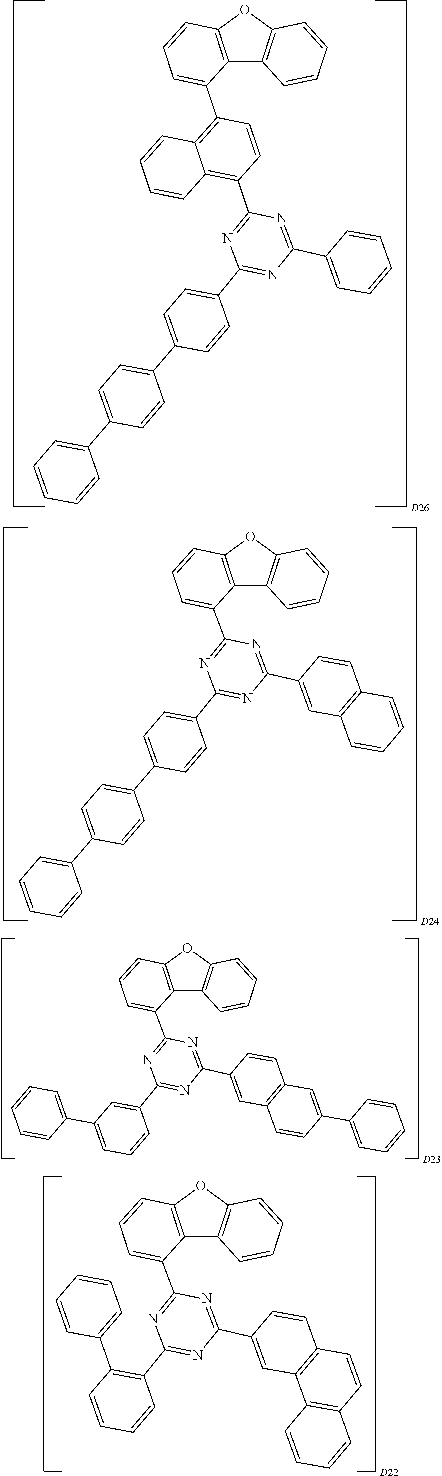

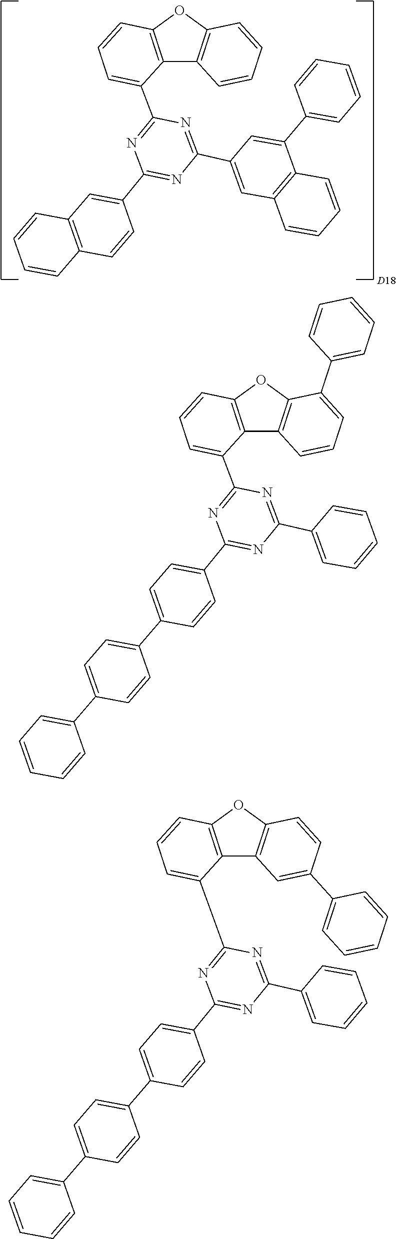

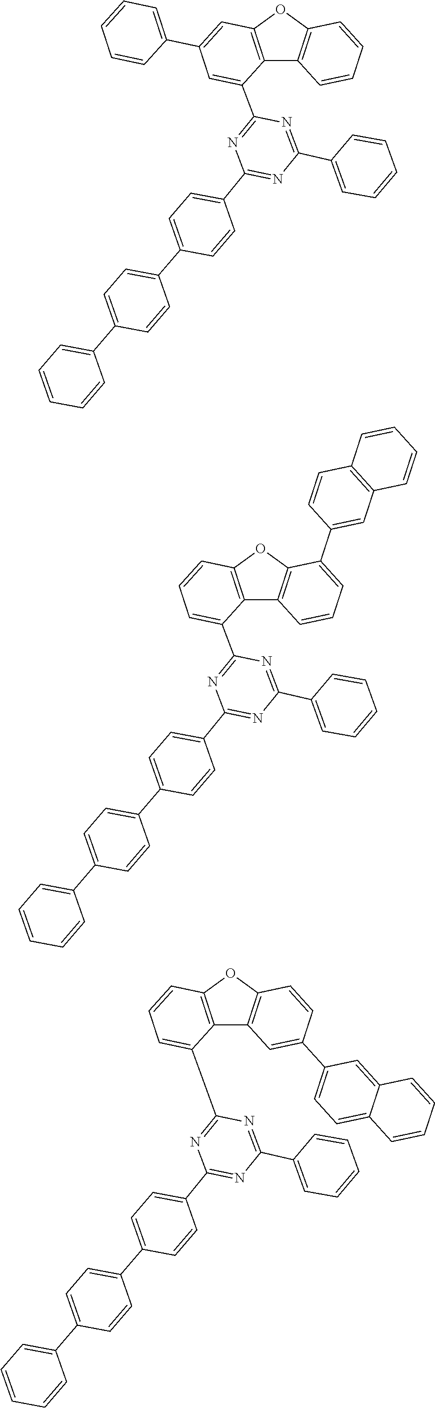 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

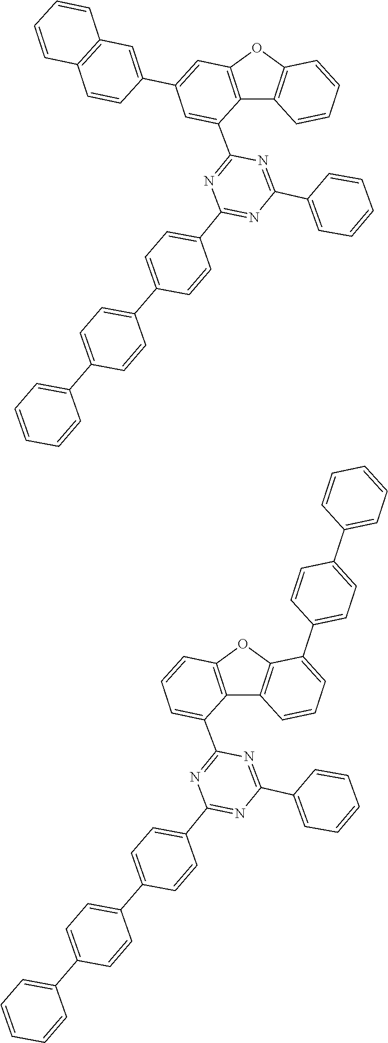 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1]

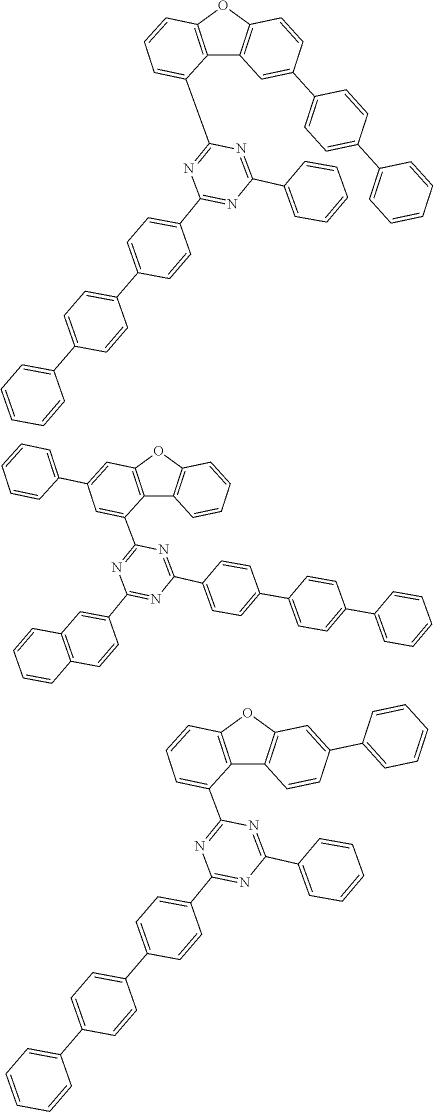

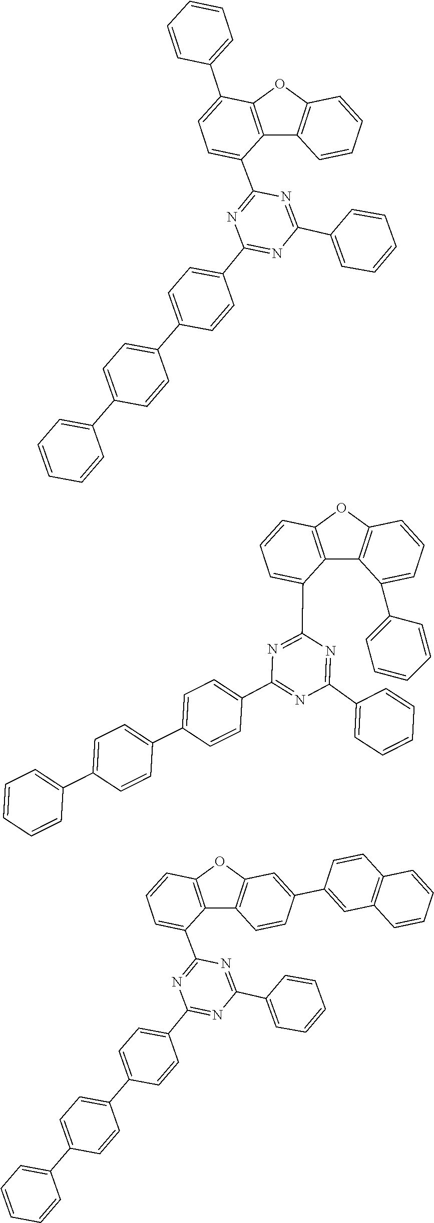 [SMILES: c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

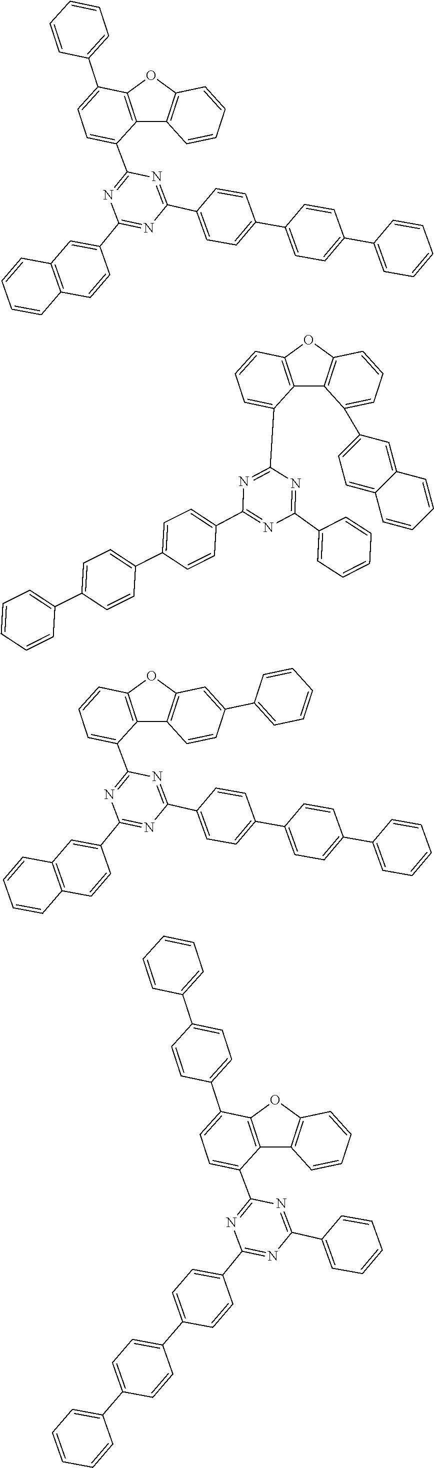 [SMILES: c1ccc(-c2ccc(-c3ccc(-c4nc(-c5ccc6ccccc6c5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccc6ccccc6c5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1]

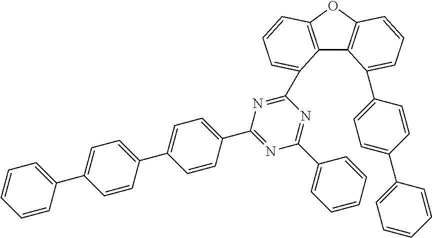 [SMILES: c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

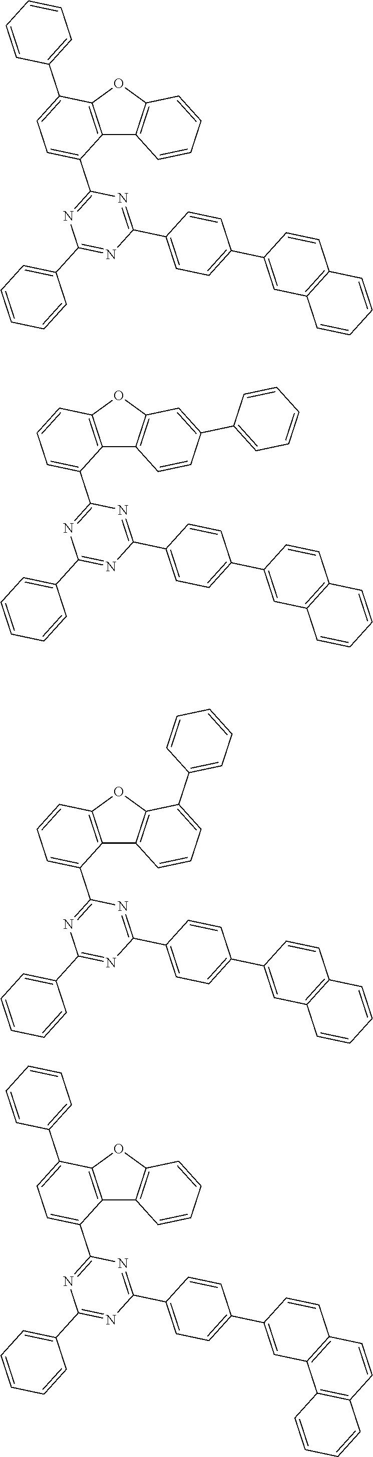

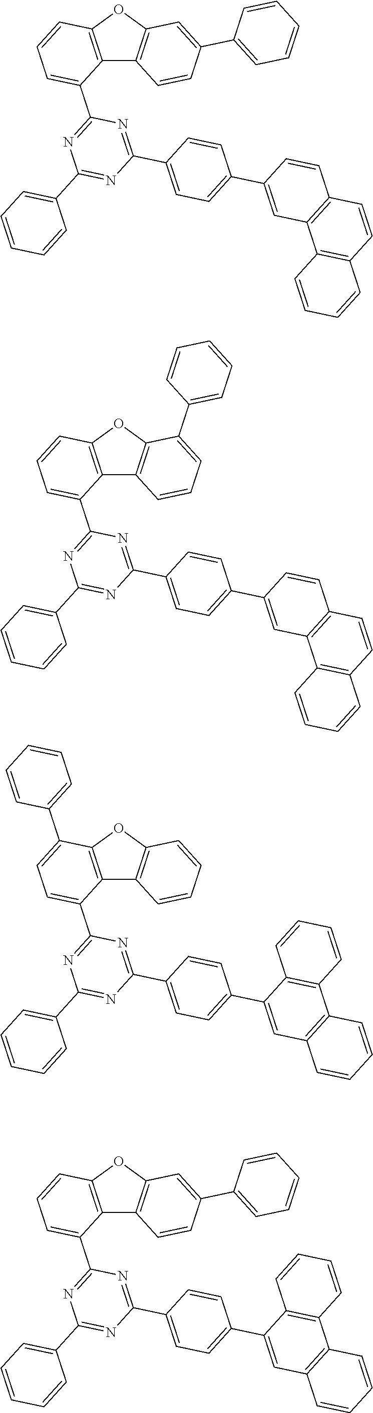 [SMILES: c1ccc(-c2ccc3c(c2)oc2cccc(-c4nc(-c5ccccc5)nc(-c5ccc(-c6ccc7ccc8ccccc8c7c6)cc5)n4)c23)cc1 ; c1ccc(-c2nc(-c3ccc(-c4ccc5ccc6ccccc6c5c4)cc3)nc(-c3cccc4oc5c(-c6ccccc6)cccc5c34)n2)cc1 ; c1ccc(-c2nc(-c3ccc(-c4cc5ccccc5c5ccccc45)cc3)nc(-c3ccc(-c4ccccc4)c4oc5ccccc5c34)n2)cc1 ; c1ccc(-c2ccc3c(c2)oc2cccc(-c4nc(-c5ccccc5)nc(-c5ccc(-c6cc7ccccc7c7ccccc67)cc5)n4)c23)cc1]

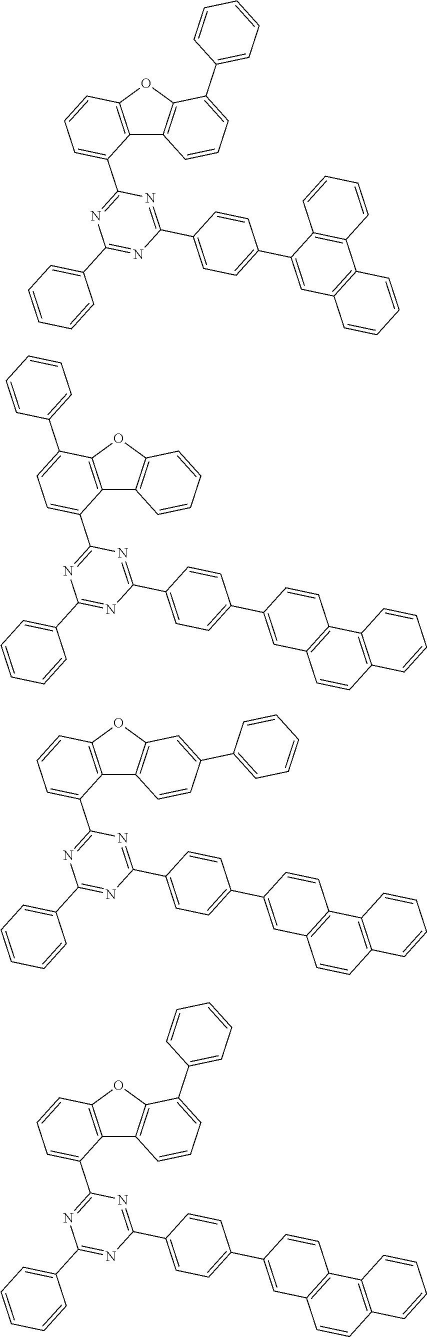

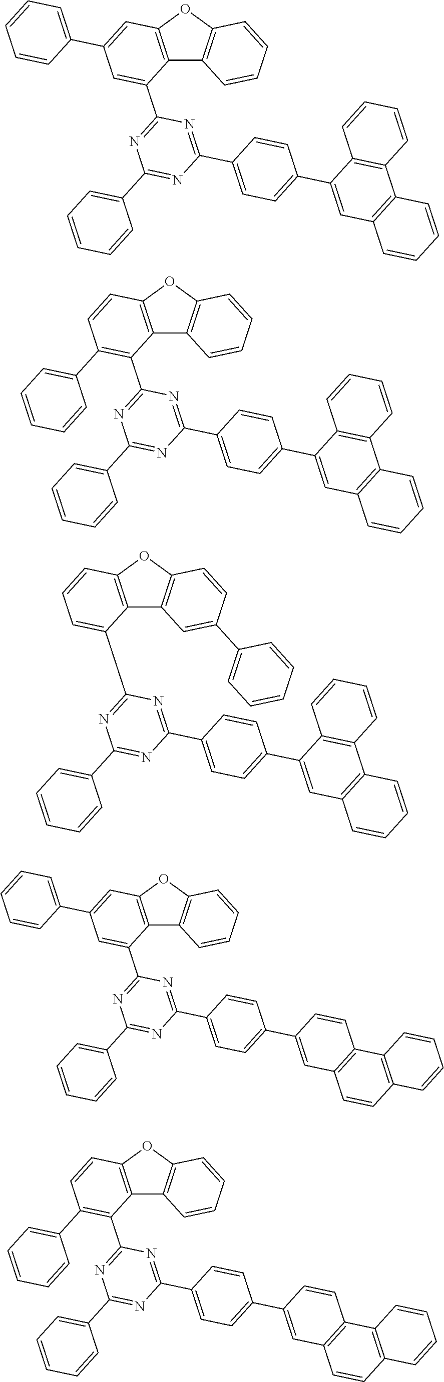

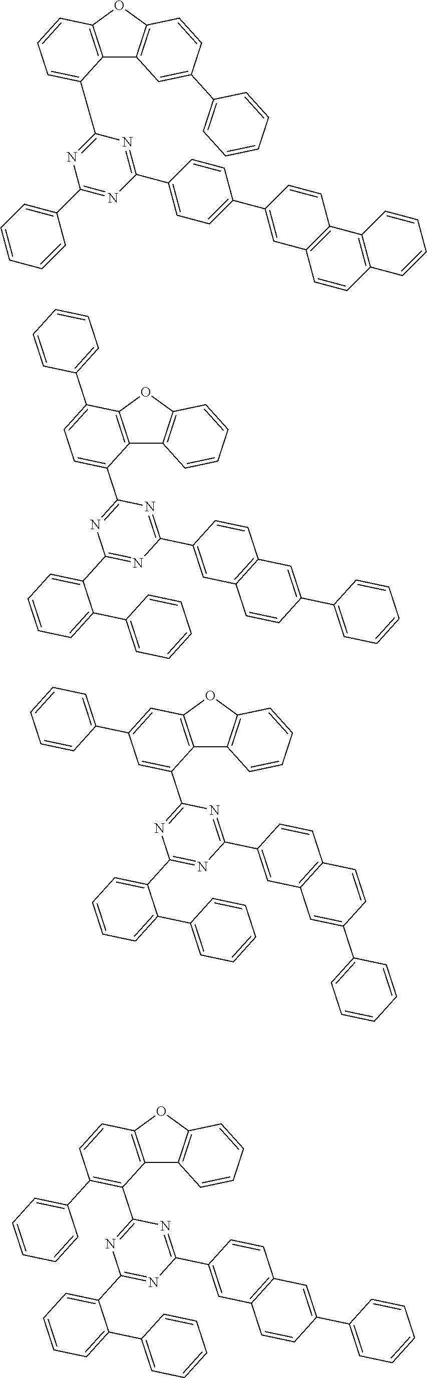

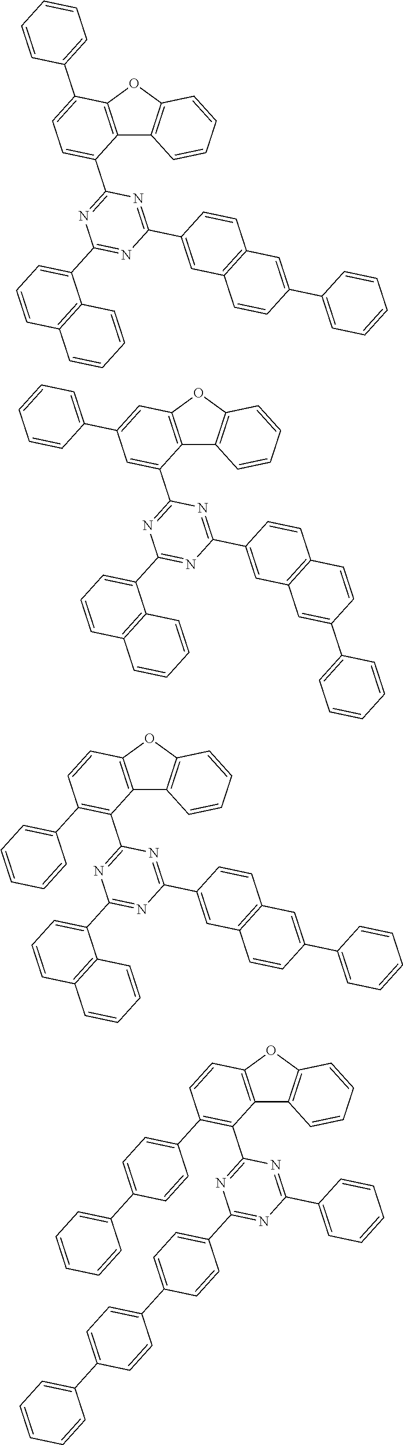

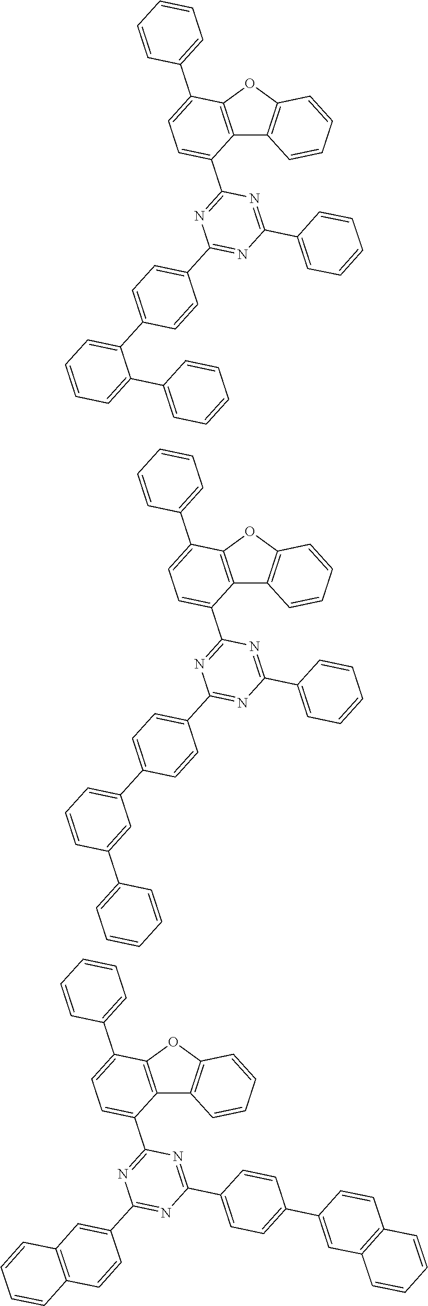

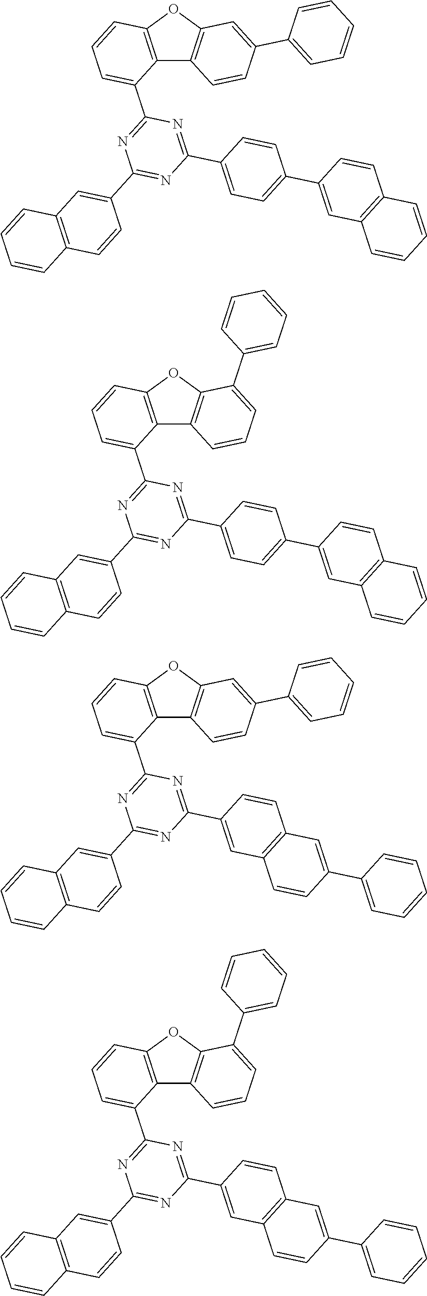 [SMILES: c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1 ; c1ccc(-c2ccc3cc(-c4nc(-c5ccc6ccccc6c5)nc(-c5cccc6oc7cc(-c8ccccc8)ccc7c56)n4)ccc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1]

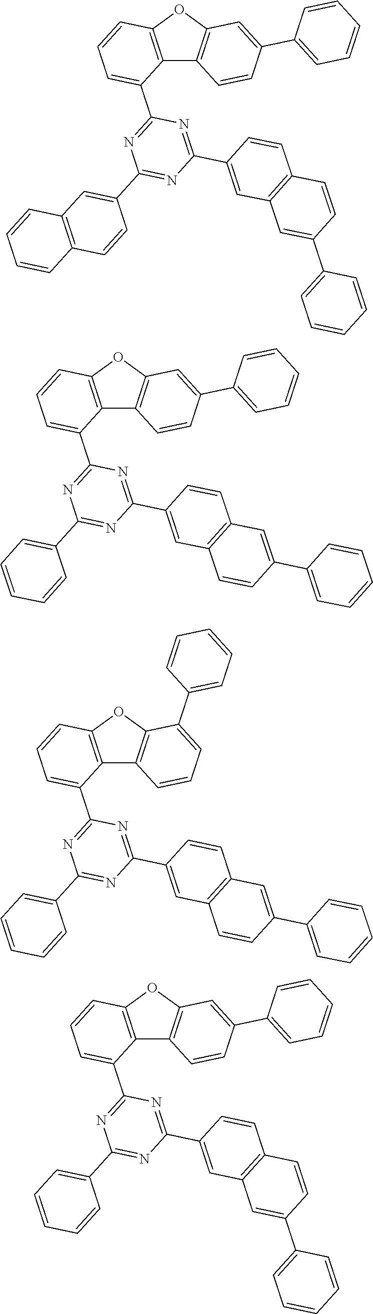

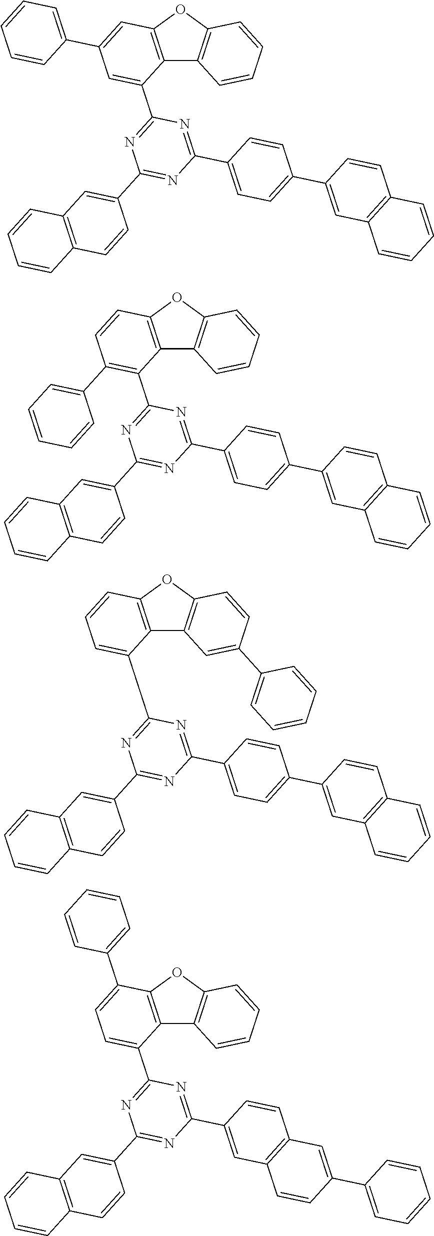

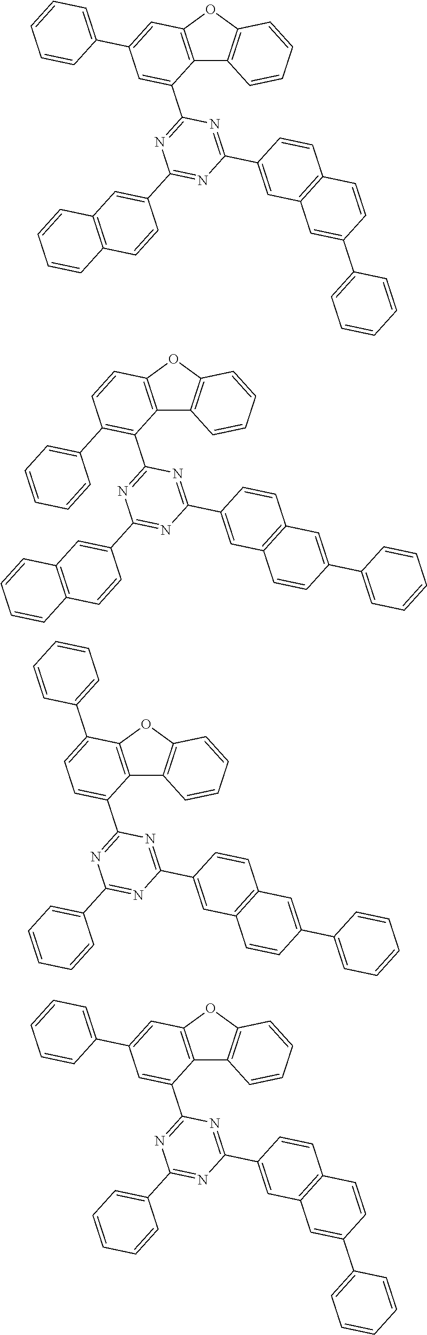

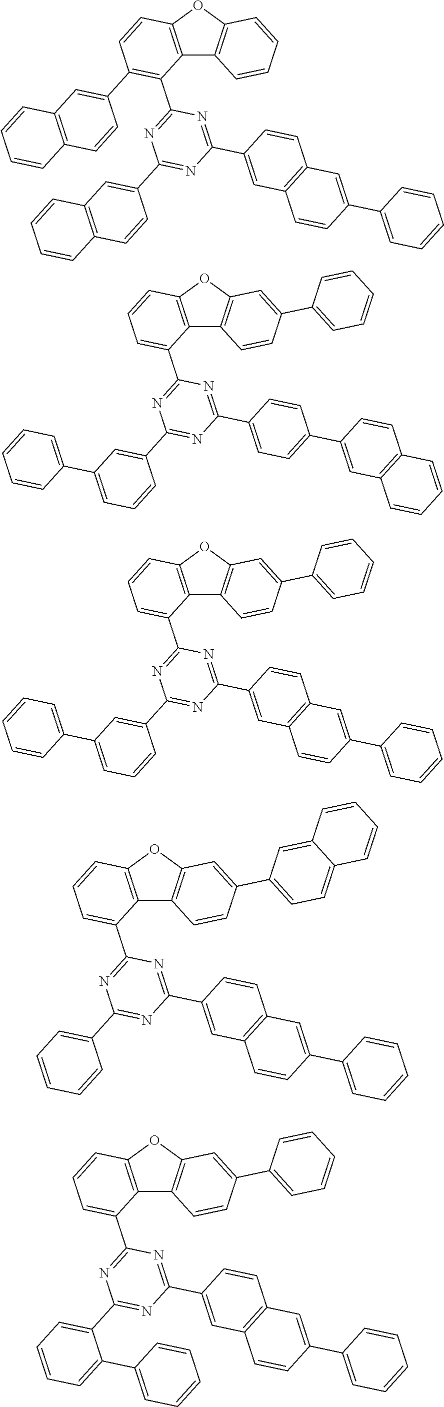

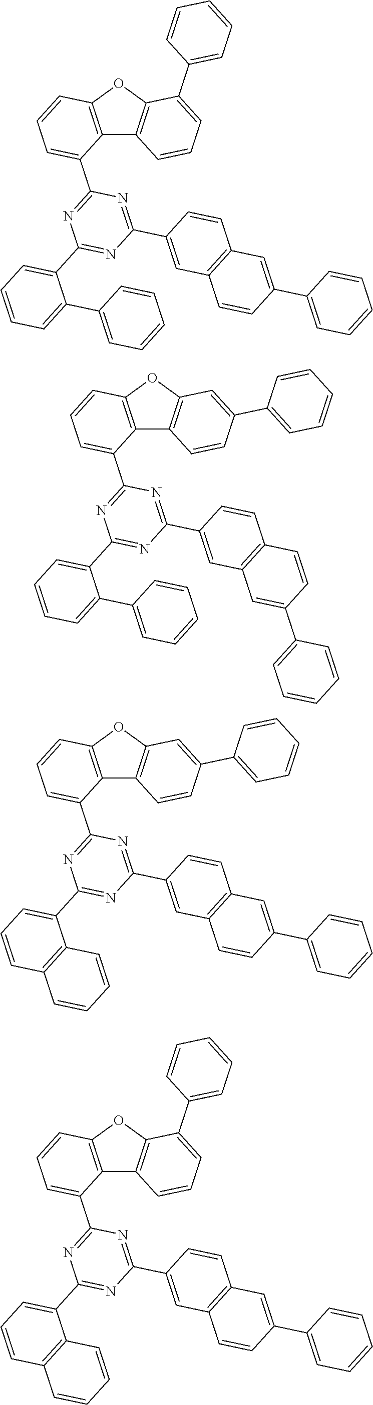

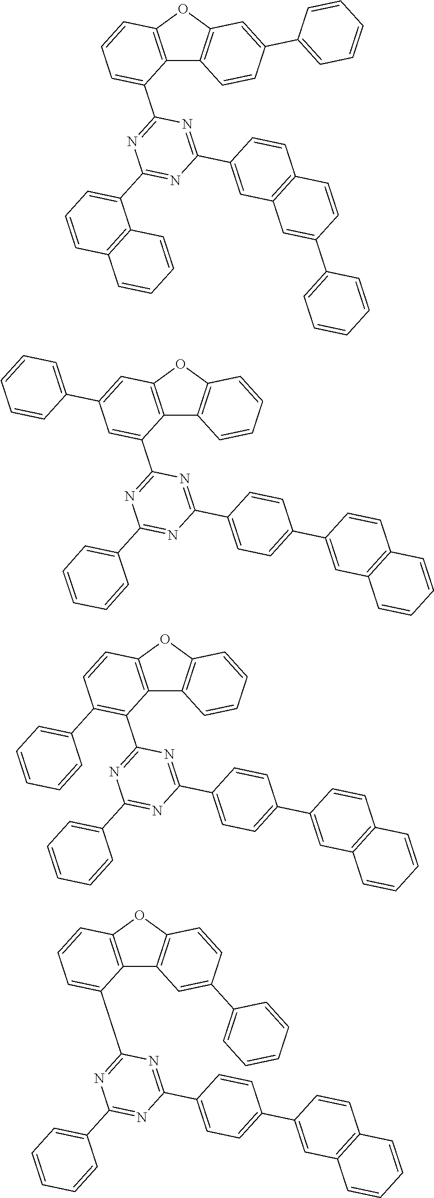

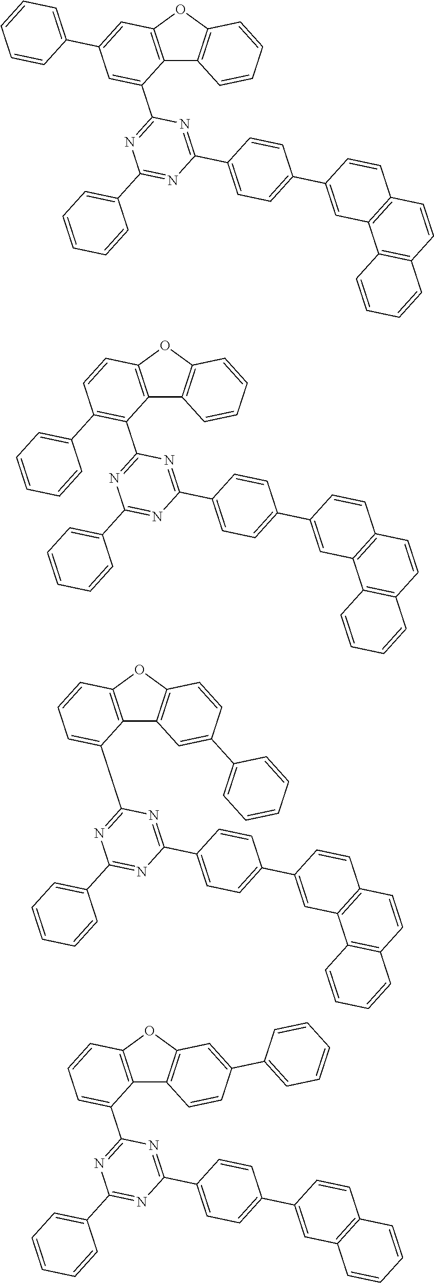

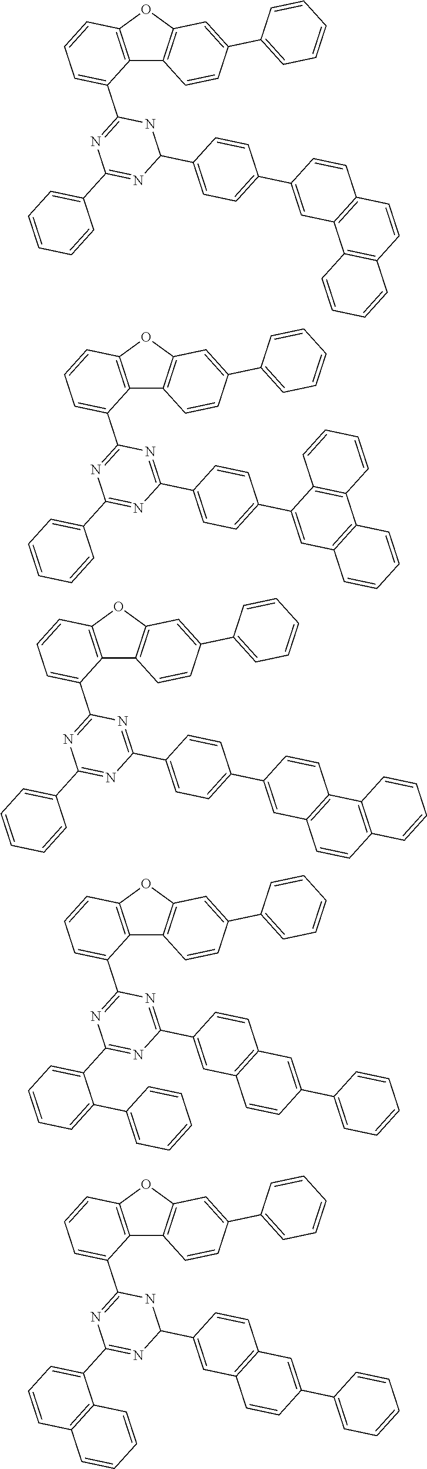

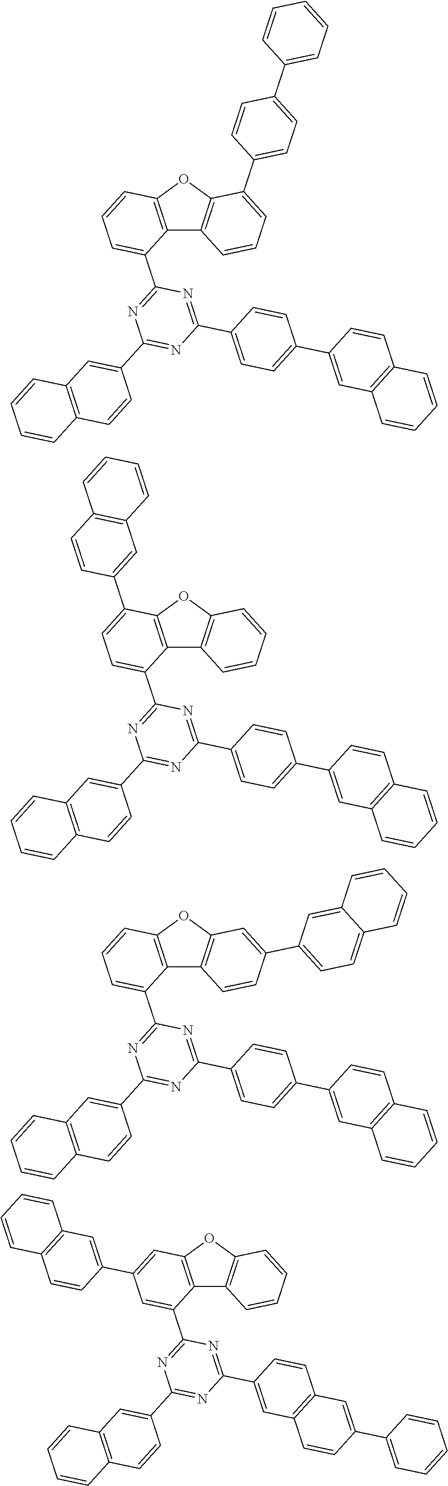

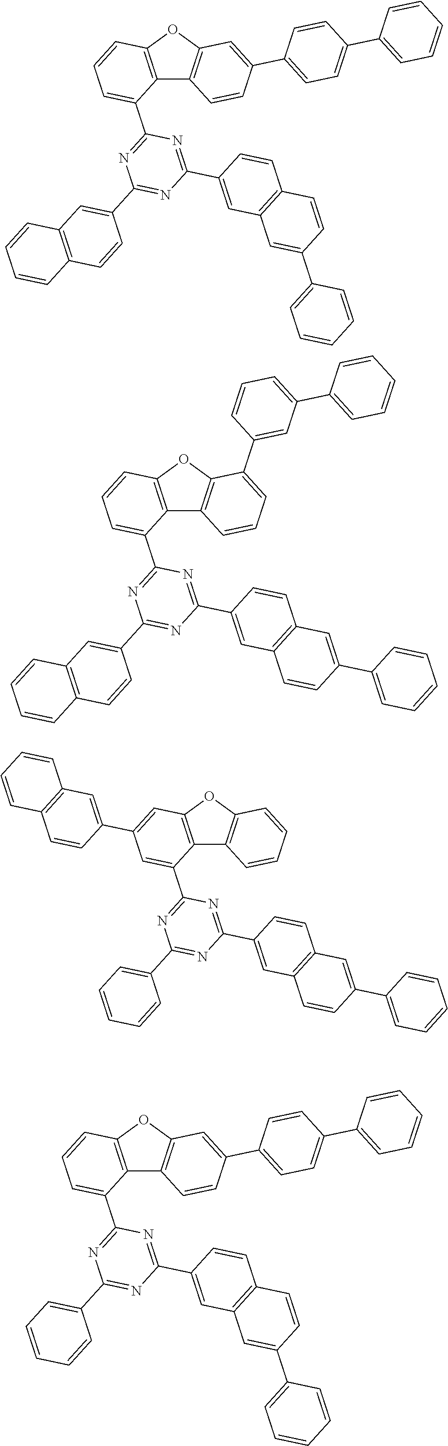

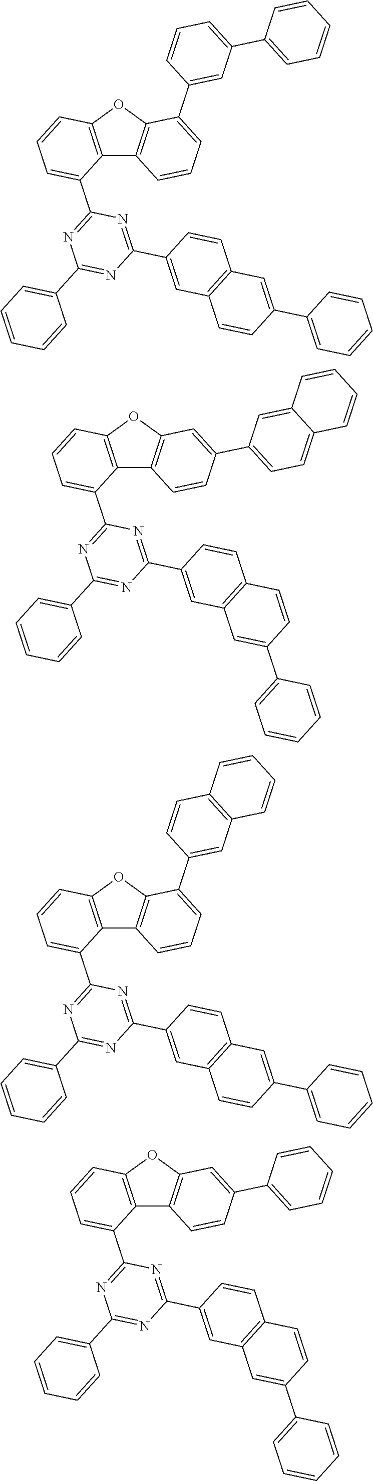

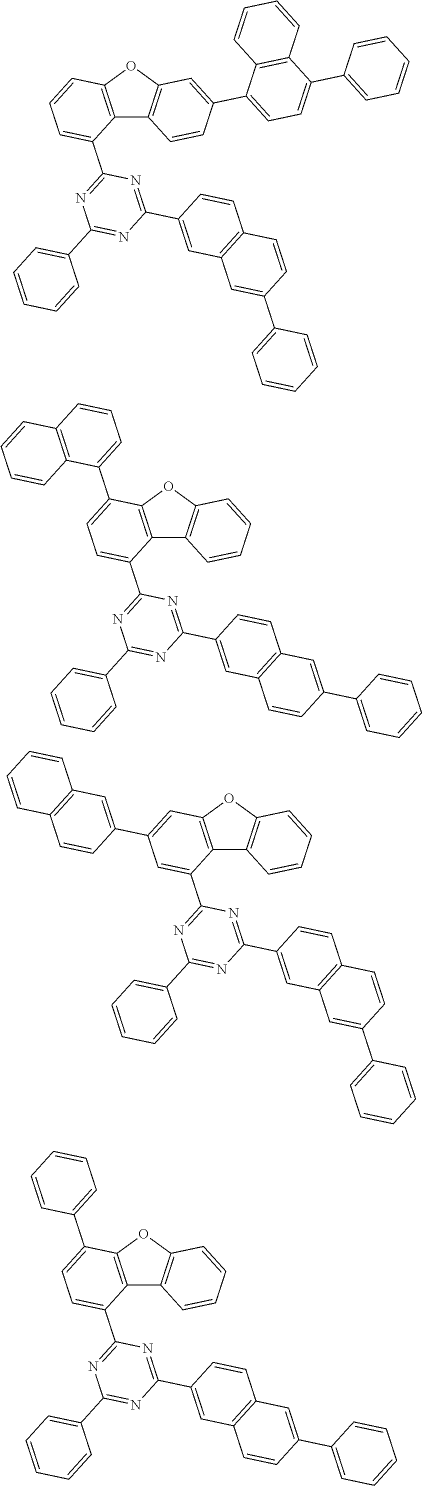

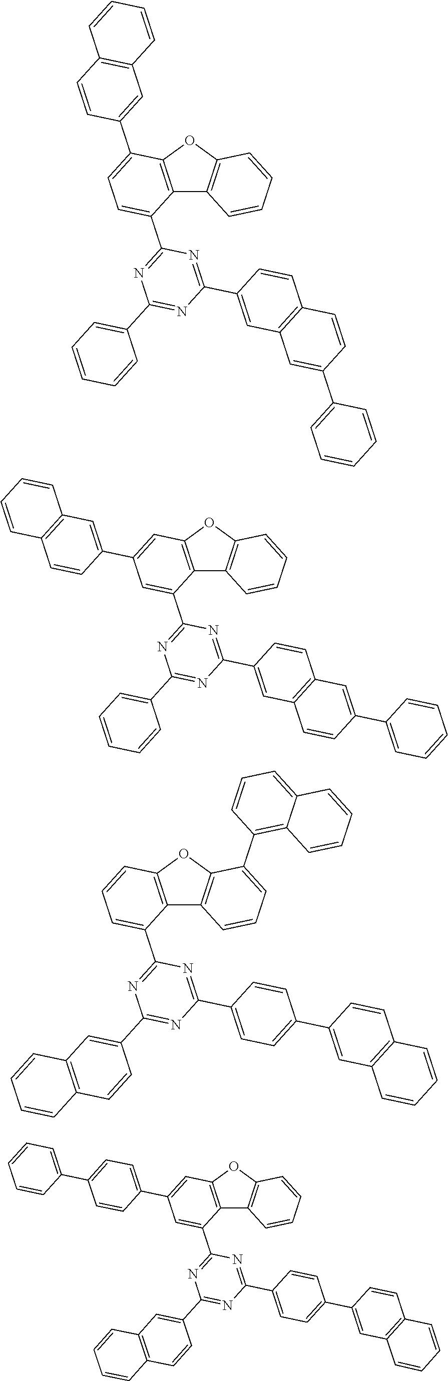

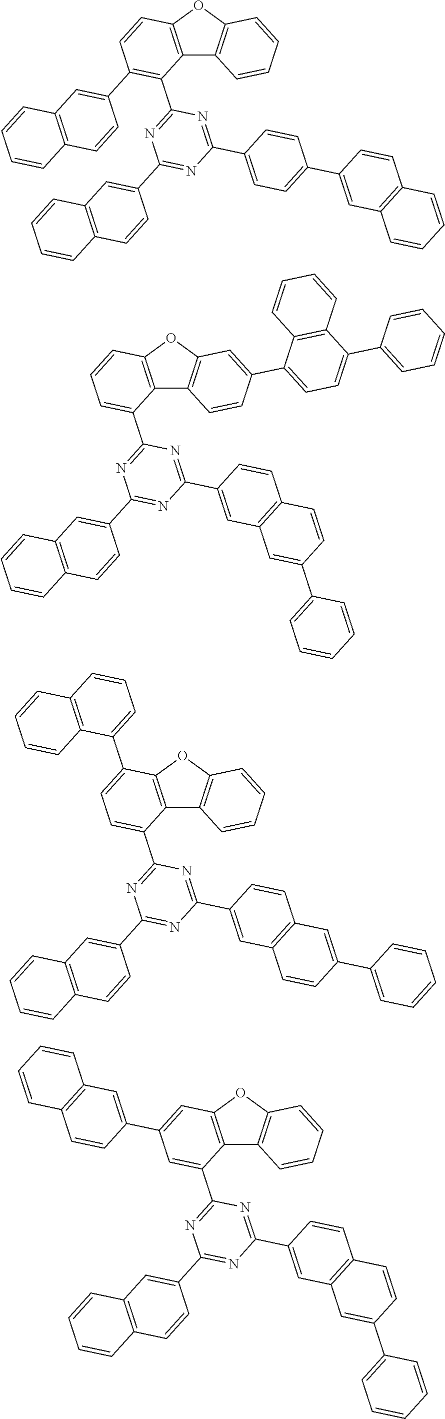

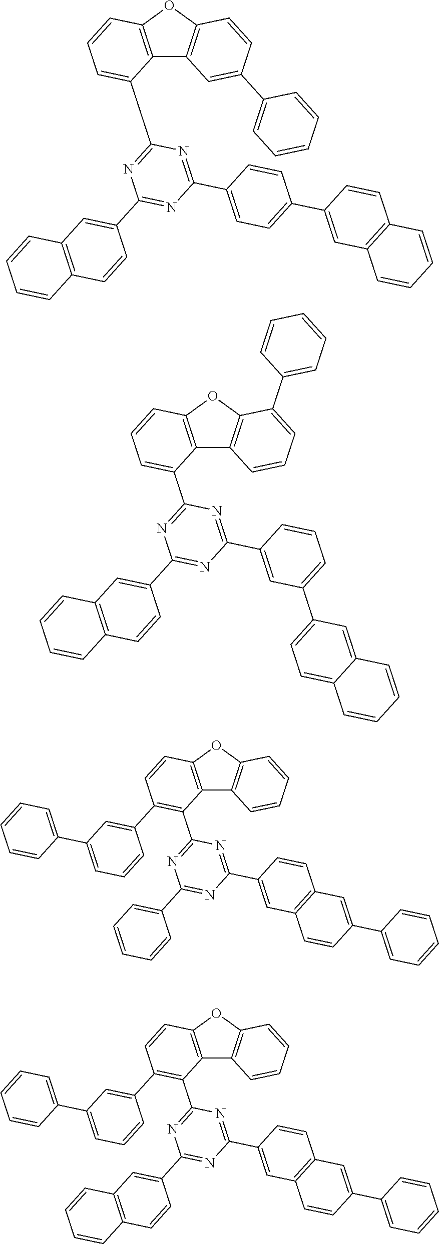 [SMILES: c1ccc(-c2ccc3oc4cccc(-c5nc(-c6ccc(-c7ccc8ccccc8c7)cc6)nc(-c6ccc7ccccc7c6)n5)c4c3c2)cc1 ; c1ccc(-c2cccc3c2oc2cccc(-c4nc(-c5cccc(-c6ccc7ccccc7c6)c5)nc(-c5ccc6ccccc6c5)n4)c23)cc1 ; c1ccc(-c2cccc(-c3ccc4oc5ccccc5c4c3-c3nc(-c4ccccc4)nc(-c4ccc5cc(-c6ccccc6)ccc5c4)n3)c2)cc1 ; c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1]

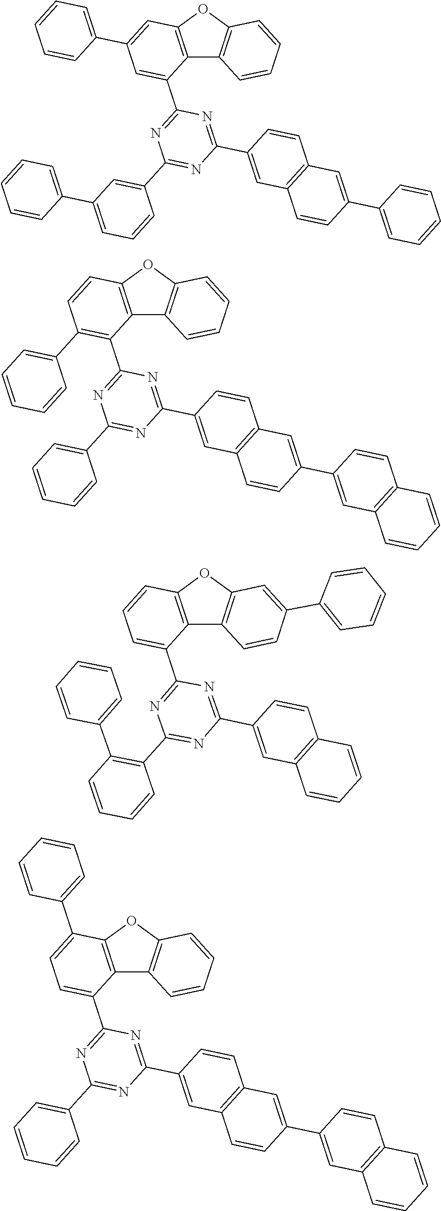

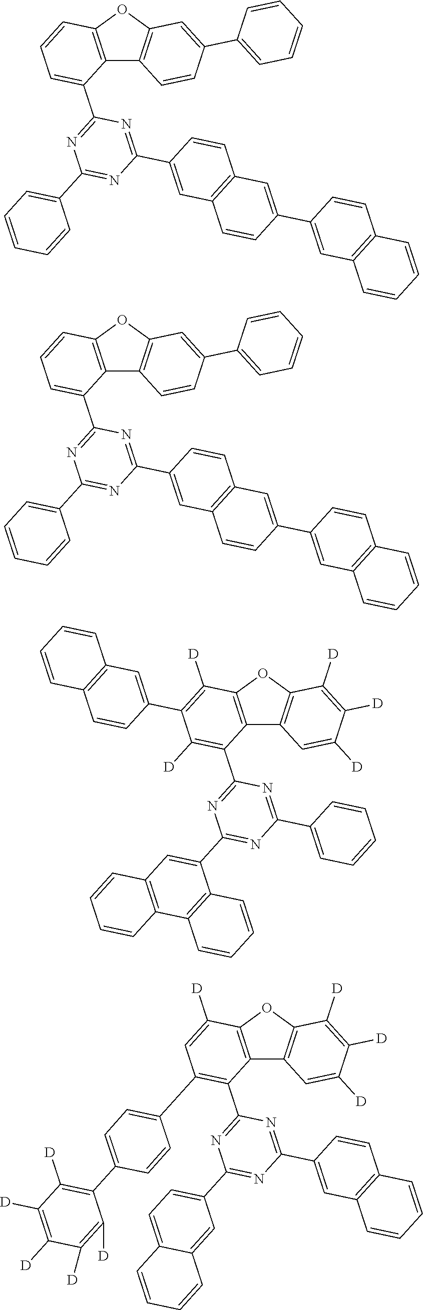

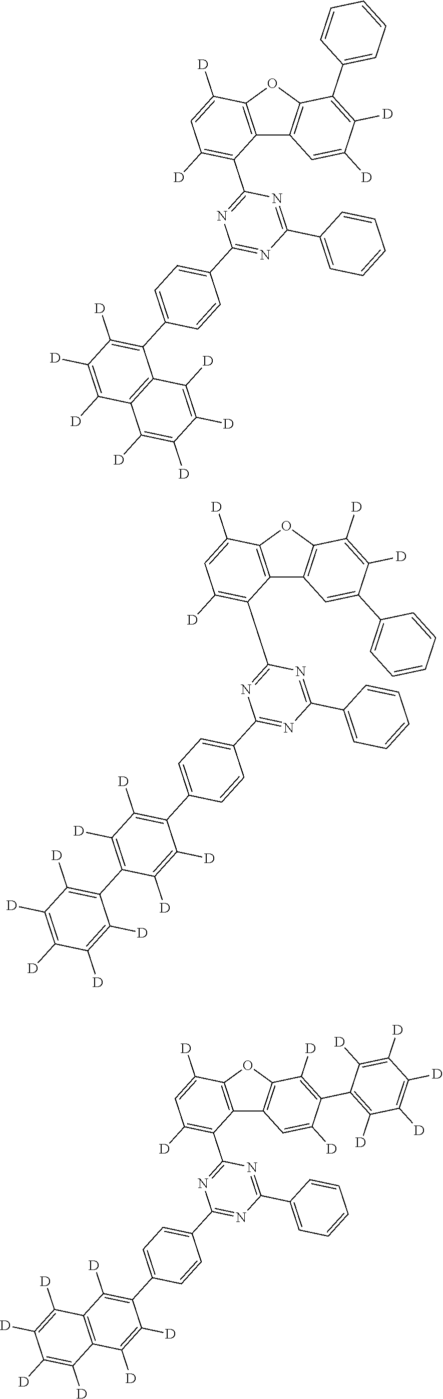

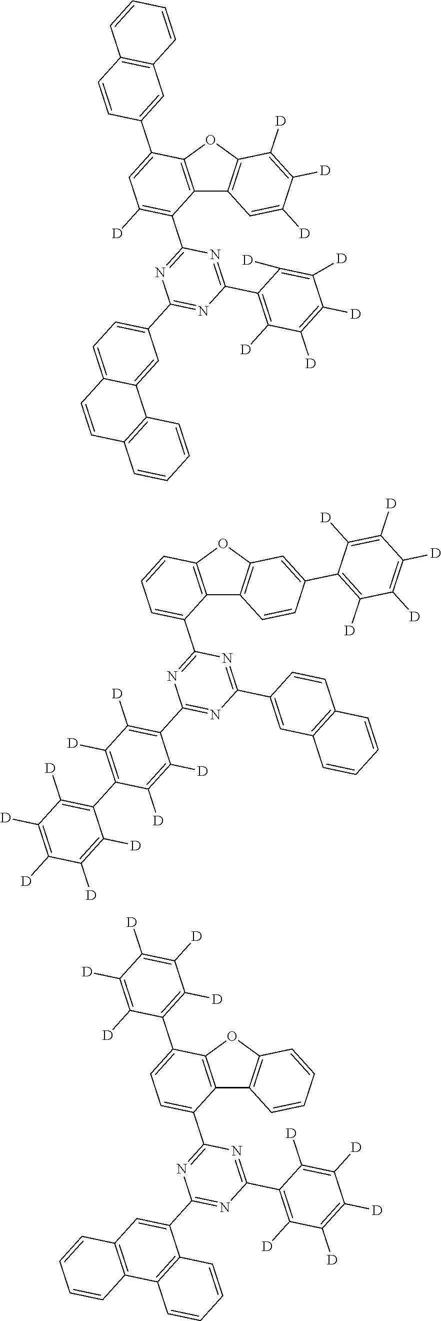 [SMILES: [2H]c1cc2c(oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c32)c([2H])c1[2H] ; [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H] ; [2H]c1c([2H])c([2H])c(-c2nc(-c3cc4ccccc4c4ccccc34)nc(-c3ccc(-c4c([2H])c([2H])c([2H])c([2H])c4[2H])c4oc5ccccc5c34)n2)c([2H])c1[2H]]

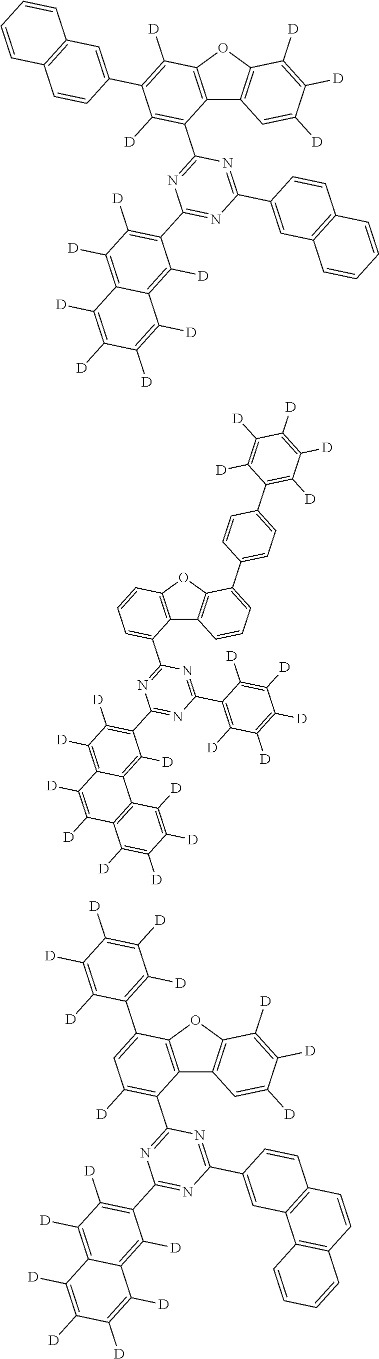

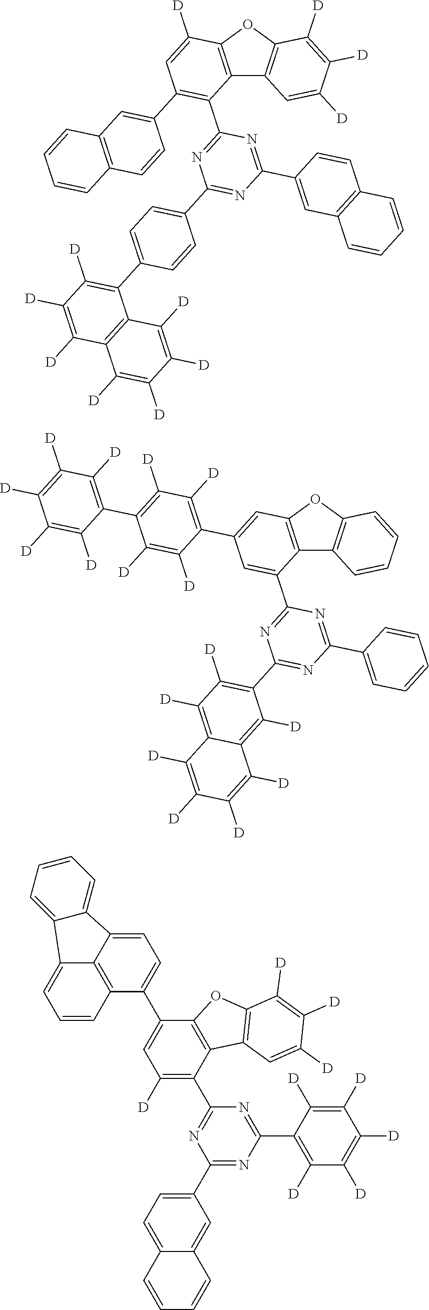

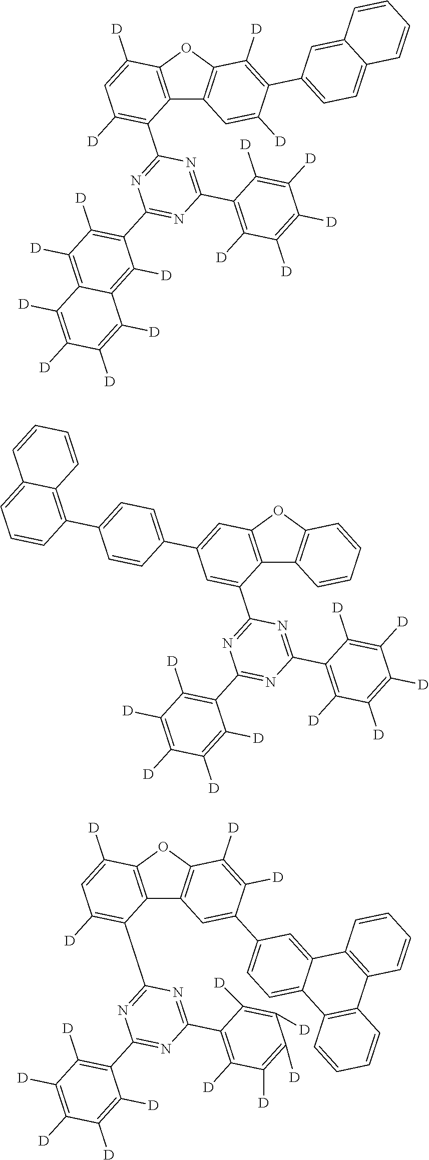

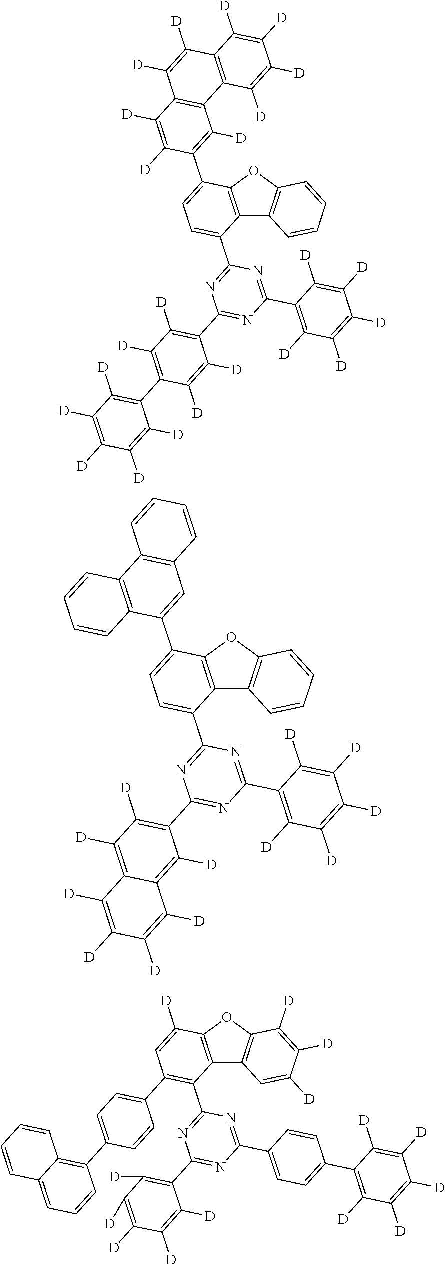

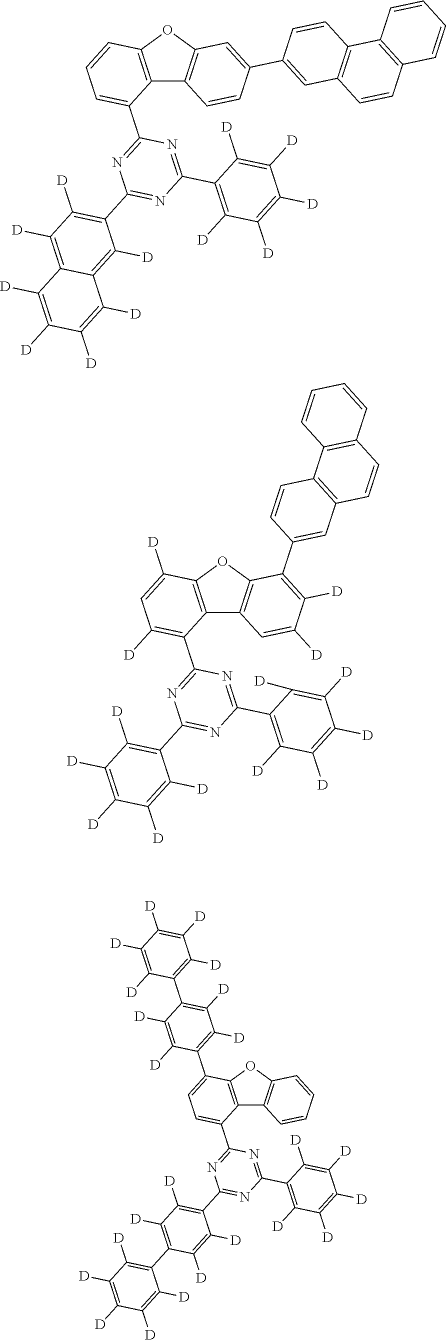

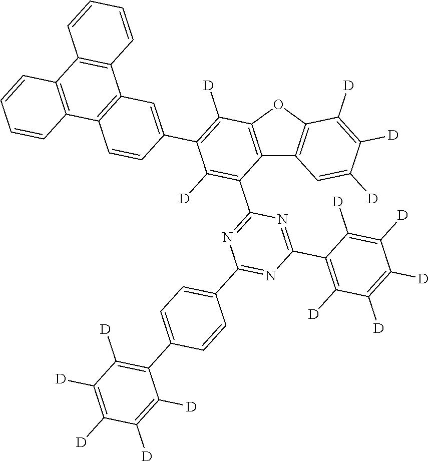 [SMILES: [2H]c1cc2c(oc3c([2H])c(-c4ccc5c6ccccc6c6ccccc6c5c4)c([2H])c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H]]

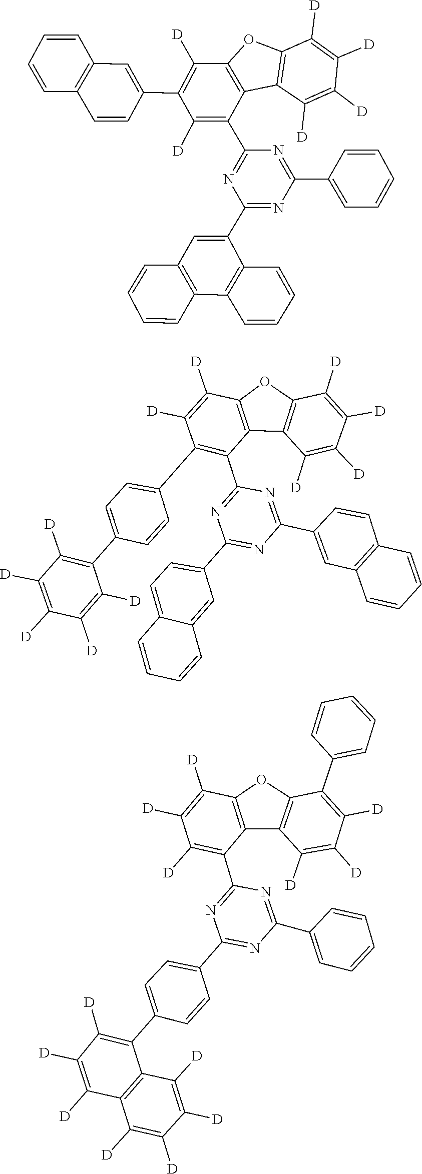

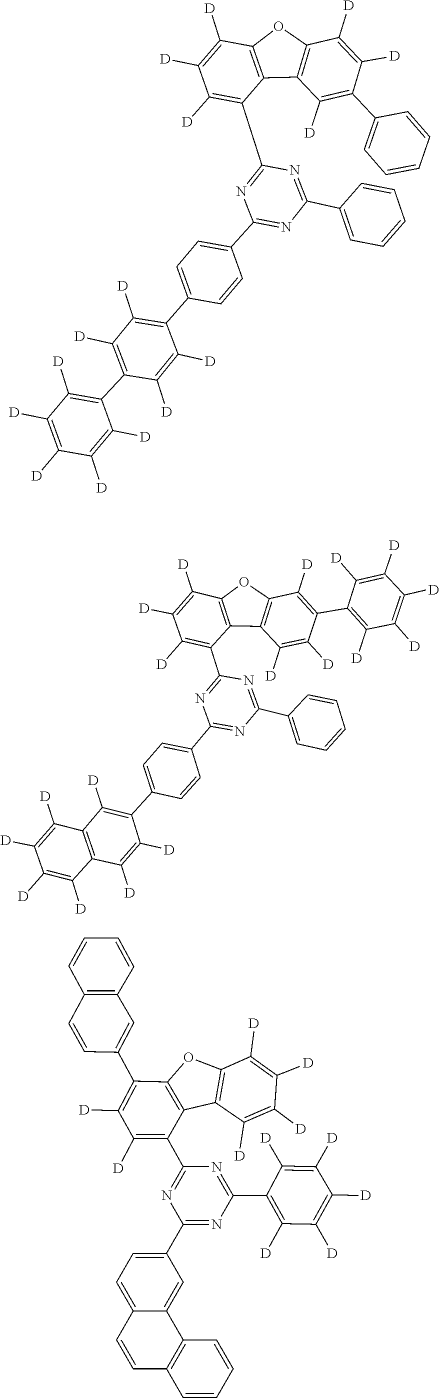

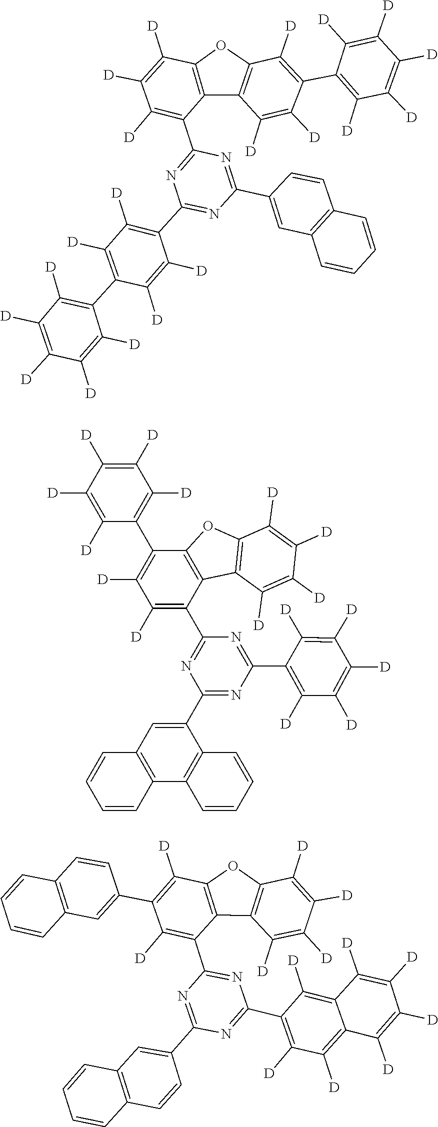

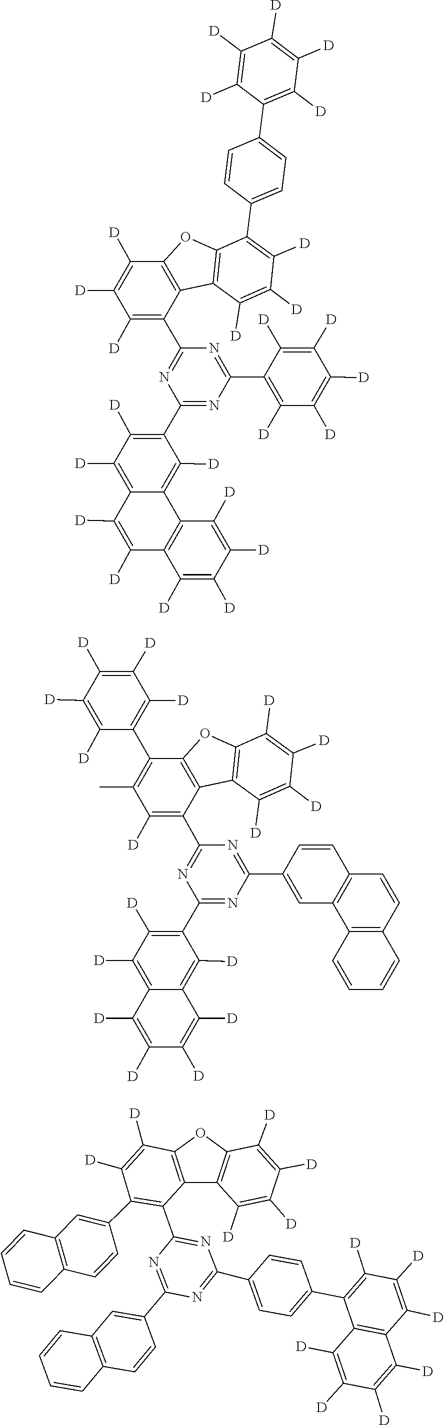

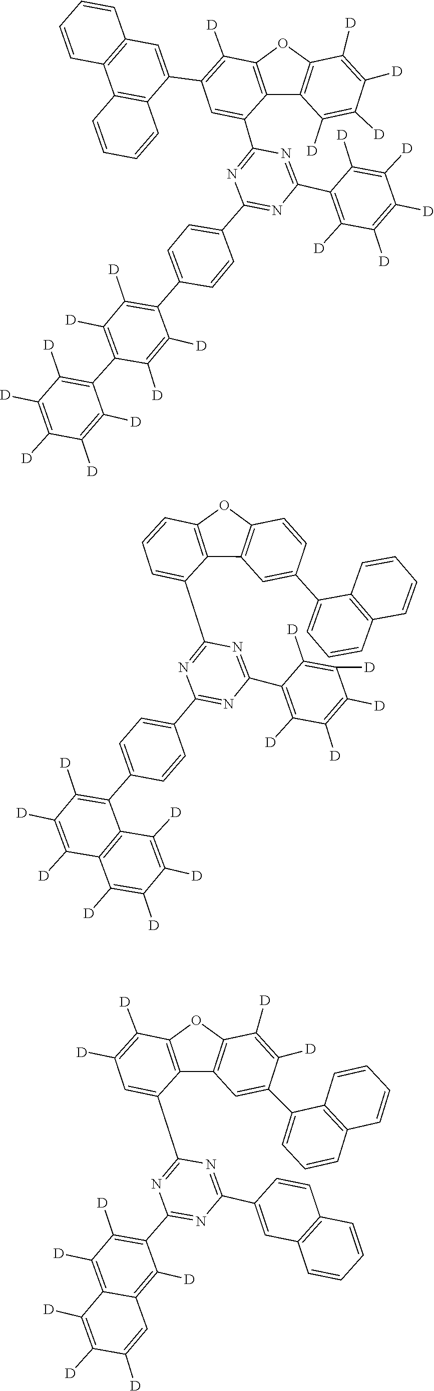 [SMILES: [2H]c1c([2H])c([2H])c(-c2nc(-c3ccc(-c4c([2H])c([2H])c(-c5c([2H])c([2H])c([2H])c([2H])c5[2H])c([2H])c4[2H])cc3)nc(-c3cc(-c4cc5ccccc5c5ccccc45)c([2H])c4oc5c([2H])c([2H])c([2H])c([2H])c5c34)n2)c([2H])c1[2H] ; [2H]c1c([2H])c([2H])c(-c2nc(-c3ccc(-c4c([2H])c([2H])c([2H])c5c([2H])c([2H])c([2H])c([2H])c45)cc3)nc(-c3cccc4oc5ccc(-c6cccc7ccccc67)cc5c34)n2)c([2H])c1[2H] ; [2H]c1cc2c([2H])c(-c3nc(-c4ccc5ccccc5c4)nc(-c4cc([2H])c([2H])c5oc6c([2H])c([2H])c(-c7cccc8ccccc78)cc6c45)n3)c([2H])c([2H])c2c([2H])c1[2H]]

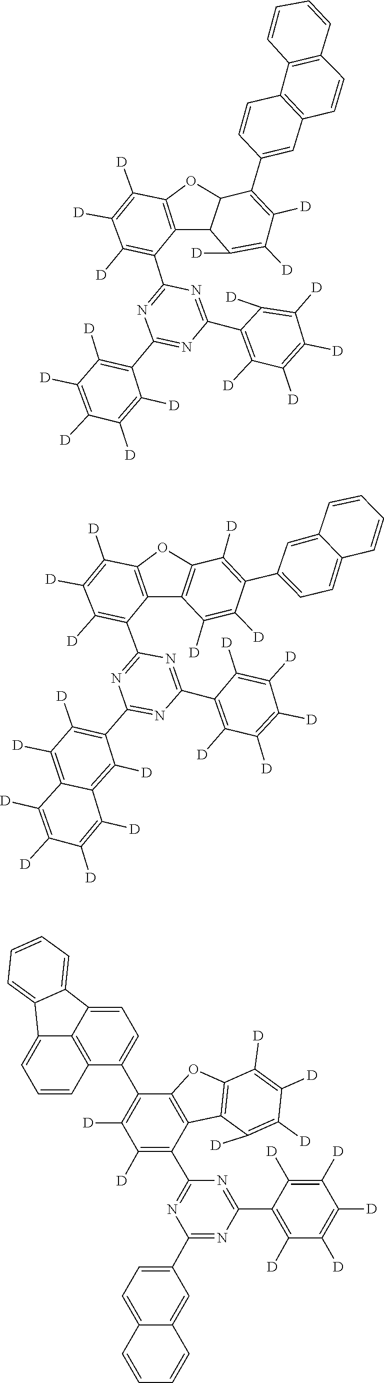 [SMILES: [2H]C1=C(c2ccc3c(ccc4ccccc43)c2)C2Oc3c([2H])c([2H])c([2H])c(-c4nc(-c5c([2H])c([2H])c([2H])c([2H])c5[2H])nc(-c5c([2H])c([2H])c([2H])c([2H])c5[2H])n4)c3C2C([2H])=C1[2H] ; [2H]c1c([2H])c([2H])c(-c2nc(-c3c([2H])c([2H])c4c([2H])c([2H])c([2H])c([2H])c4c3[2H])nc(-c3c([2H])c([2H])c([2H])c4oc5c([2H])c(-c6ccc7ccccc7c6)c([2H])c([2H])c5c34)n2)c([2H])c1[2H] ; [2H]c1c([2H])c([2H])c(-c2nc(-c3ccc4ccccc4c3)nc(-c3c([2H])c([2H])c(-c4ccc5c6c(cccc46)-c4ccccc4-5)c4oc5c([2H])c([2H])c([2H])c([2H])c5c34)n2)c([2H])c1[2H]]

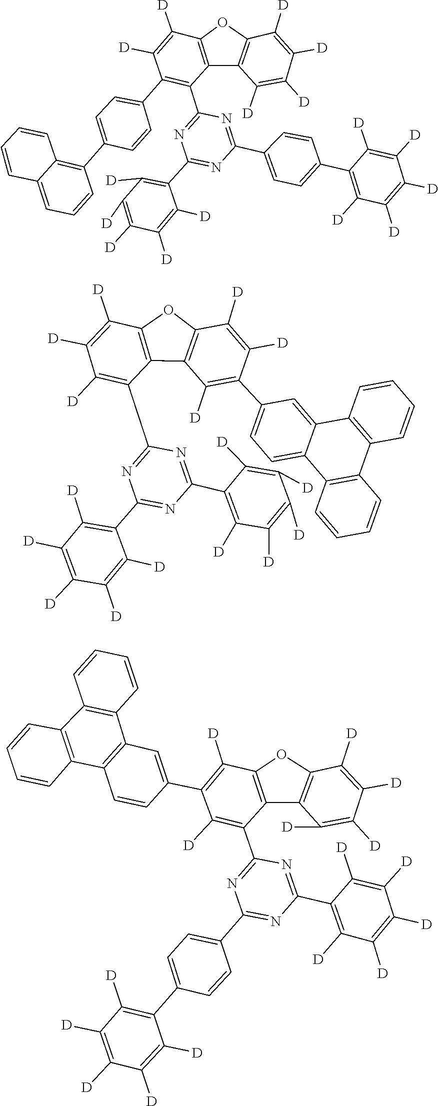

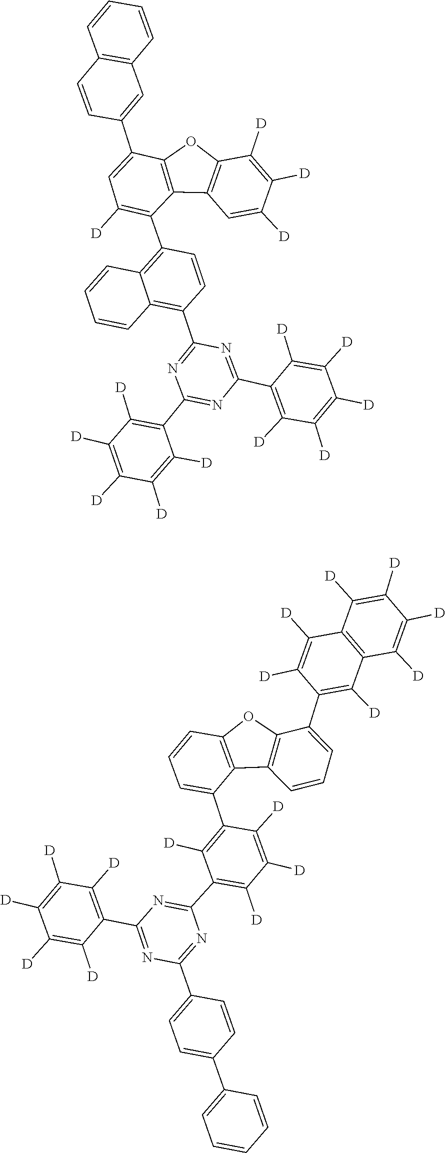 [SMILES: [2H]c1cc2c(oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H] ; [2H]c1c([2H])c([2H])c(-c2nc(-c3ccc(-c4ccccc4)cc3)nc(-c3c([2H])c([2H])c([2H])c(-c4cccc5oc6c(-c7c([2H])c([2H])c8c([2H])c([2H])c([2H])c([2H])c8c7[2H])cccc6c45)c3[2H])n2)c([2H])c1[2H]]

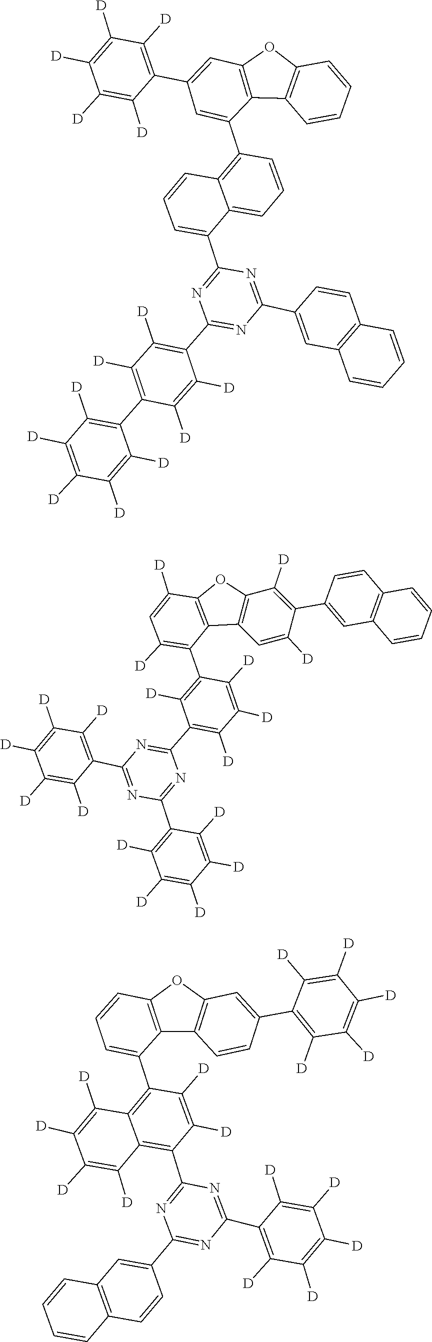

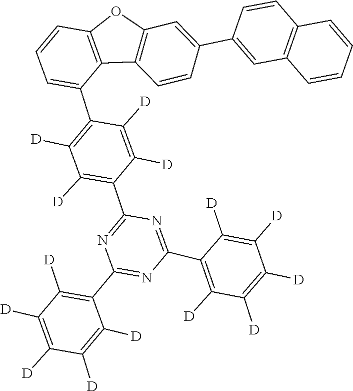 [SMILES: [2H]c1c([2H])c([2H])c(-c2nc(-c3c([2H])c([2H])c([2H])c([2H])c3[2H])nc(-c3c([2H])c([2H])c(-c4cccc5oc6cc(-c7ccc8ccccc8c7)ccc6c45)c([2H])c3[2H])n2)c([2H])c1[2H]]

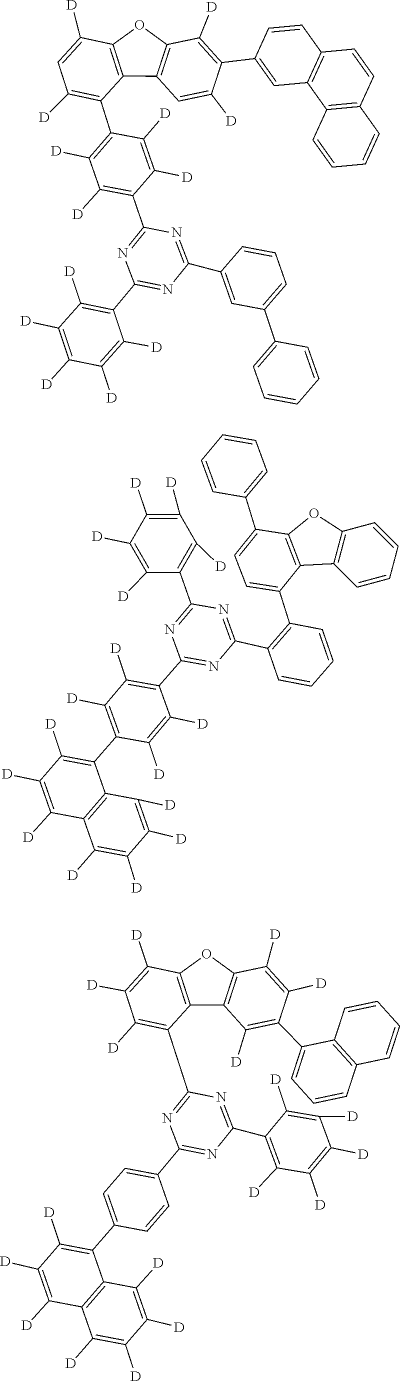

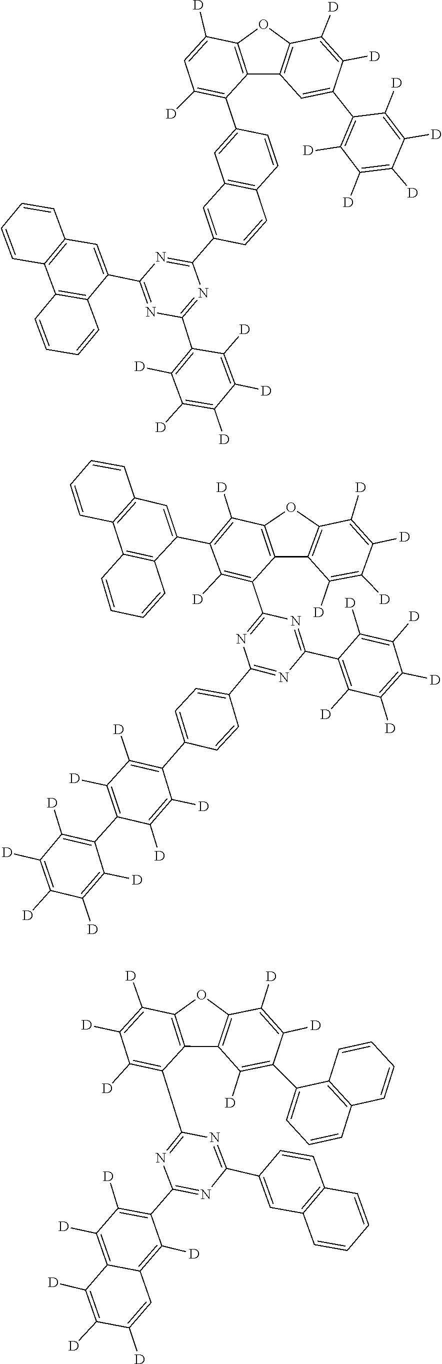

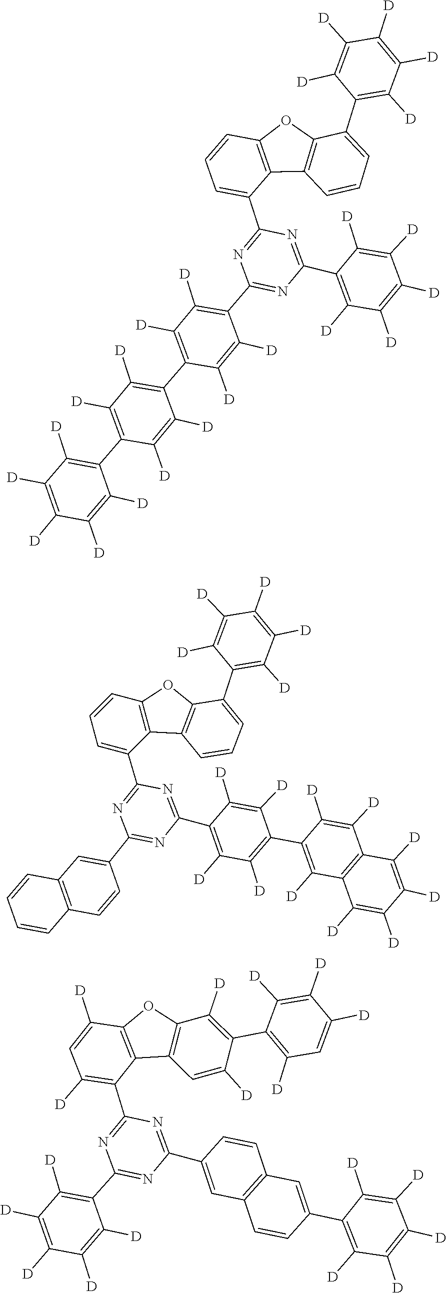

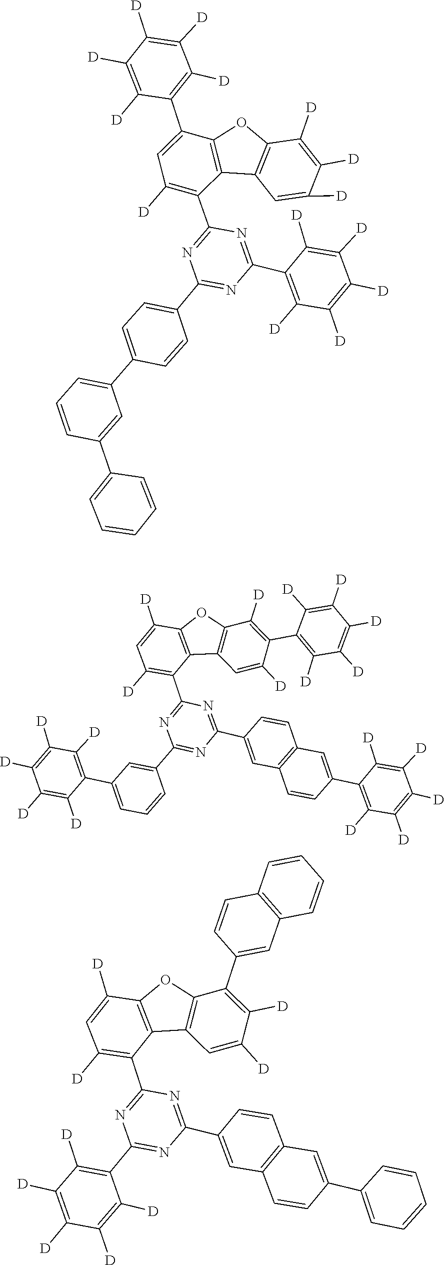

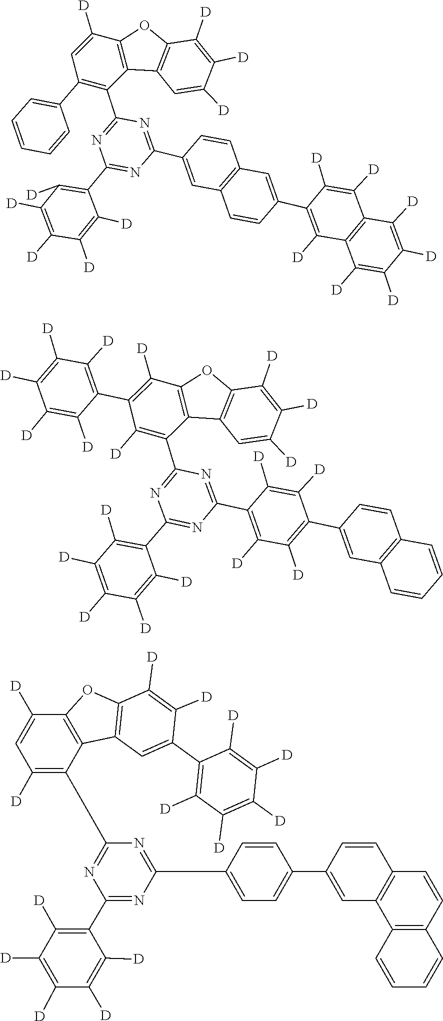

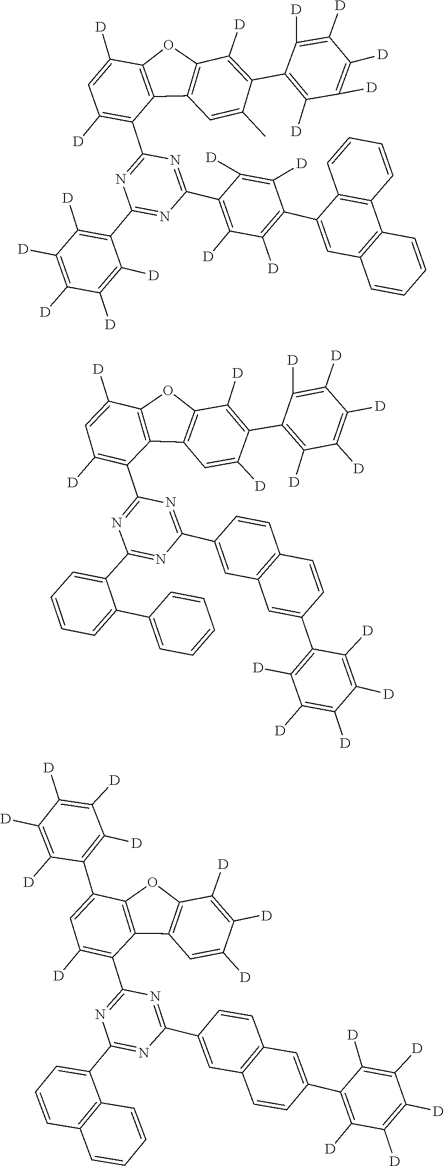

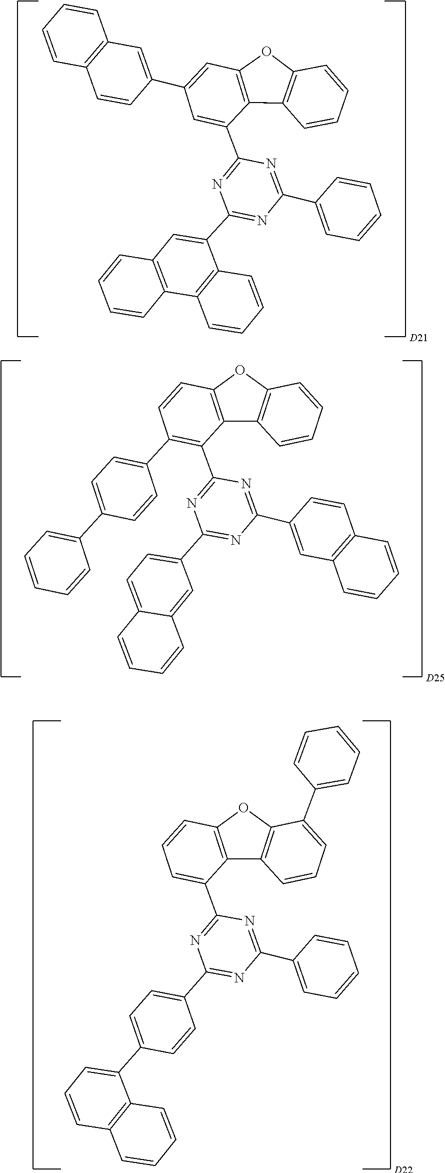

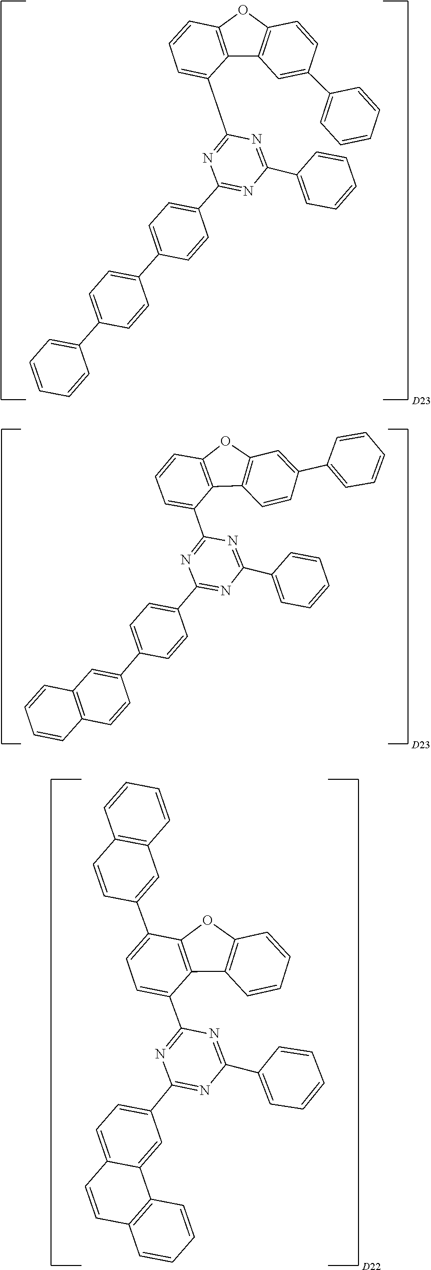

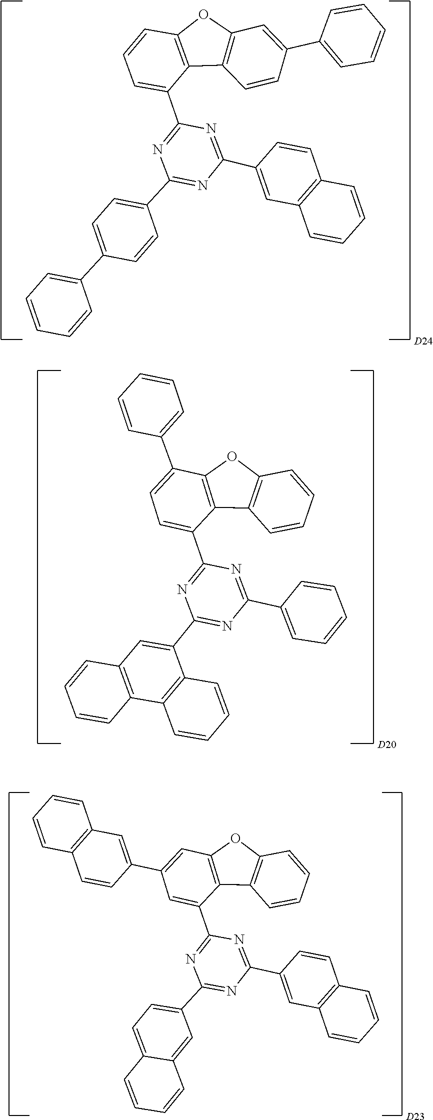

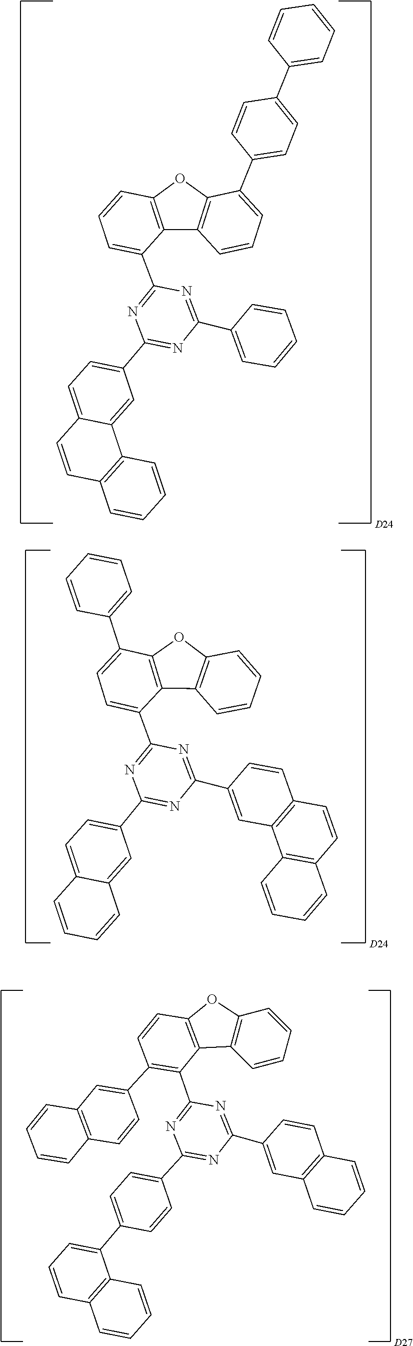

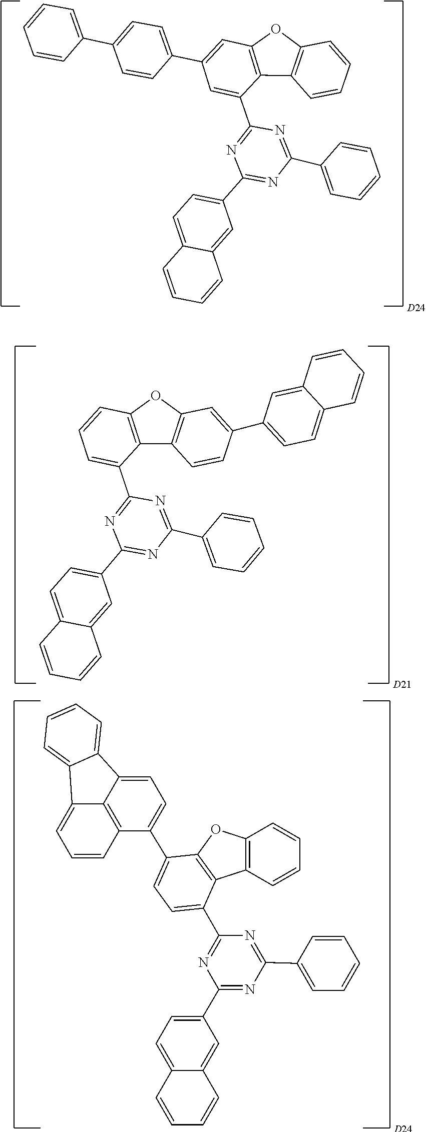

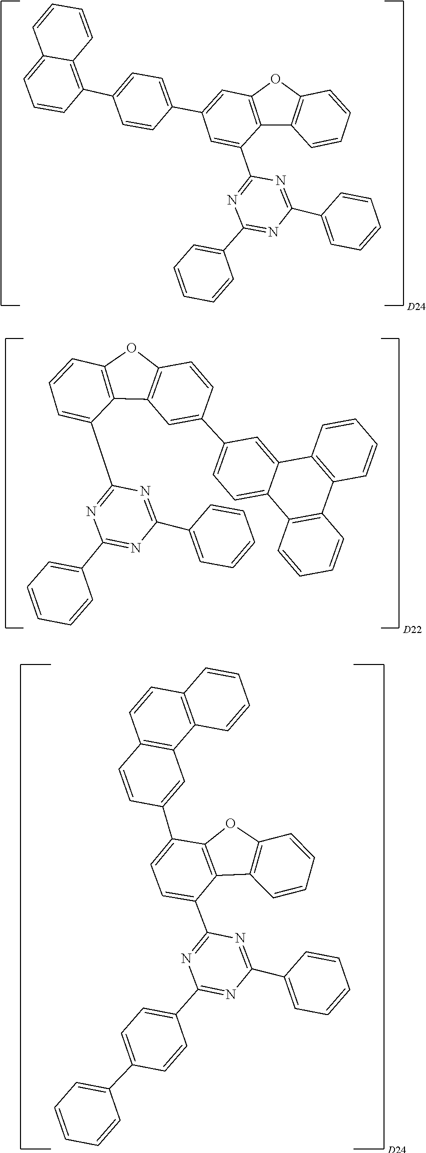

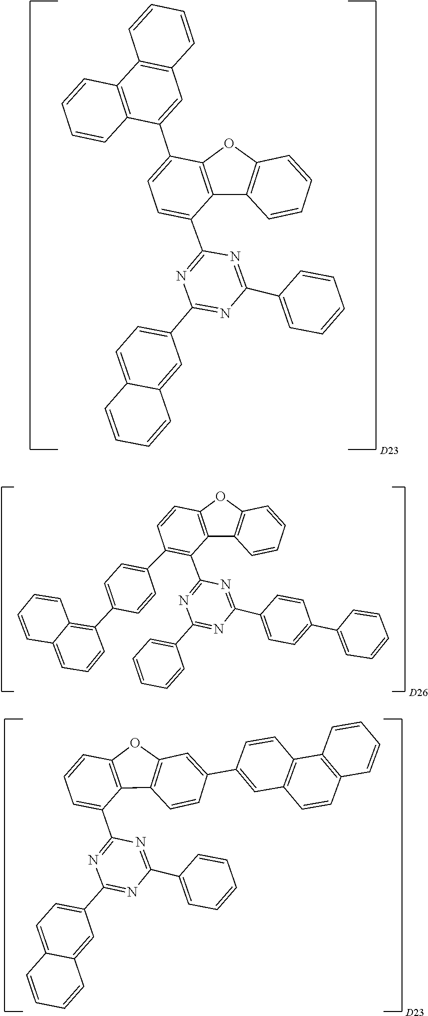

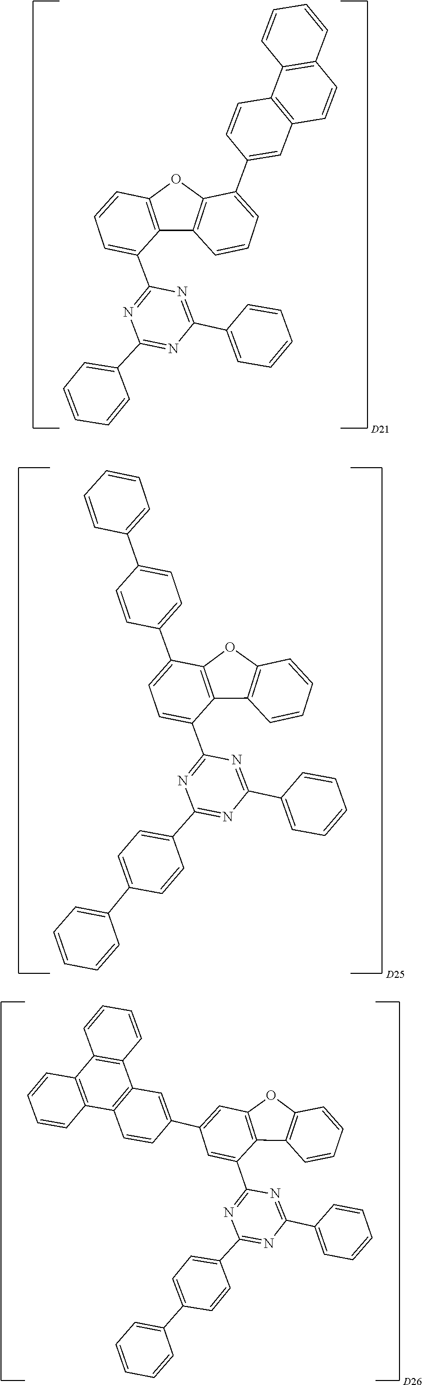

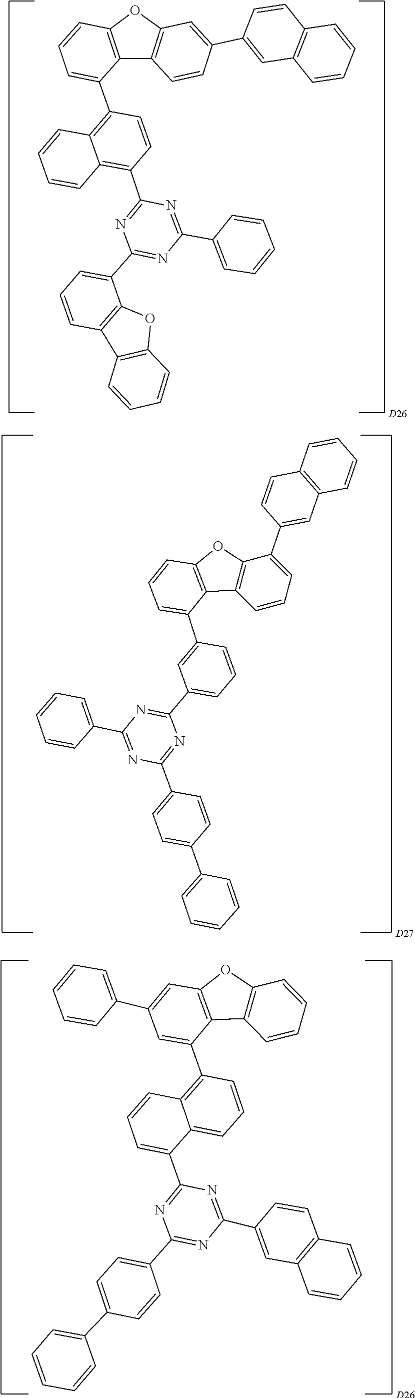

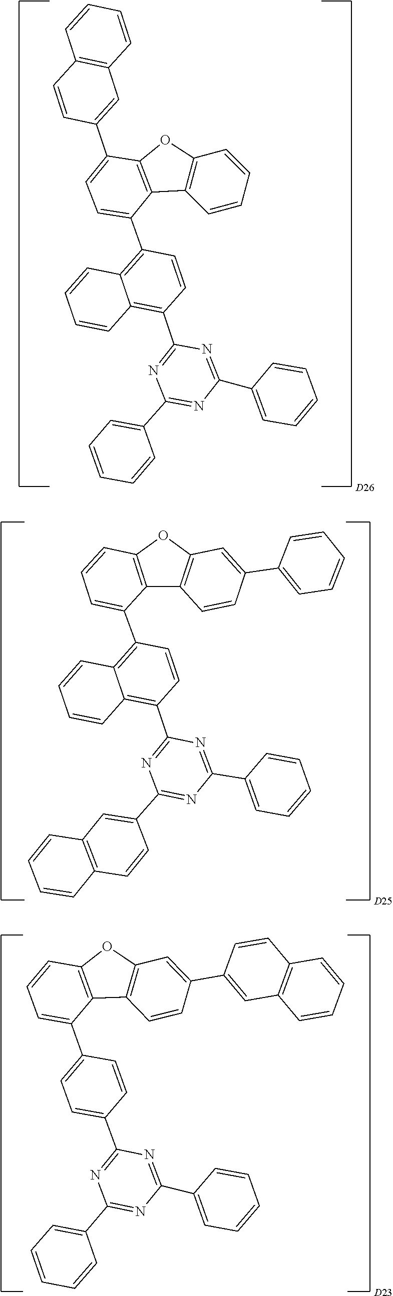

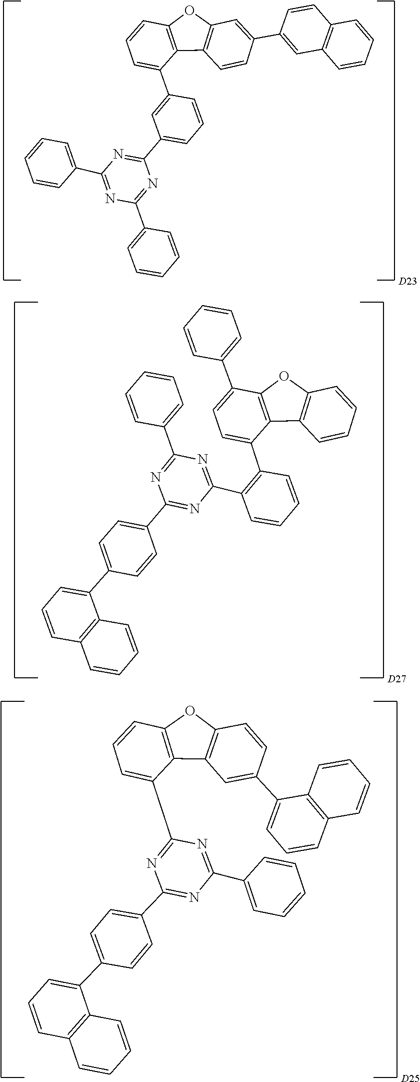

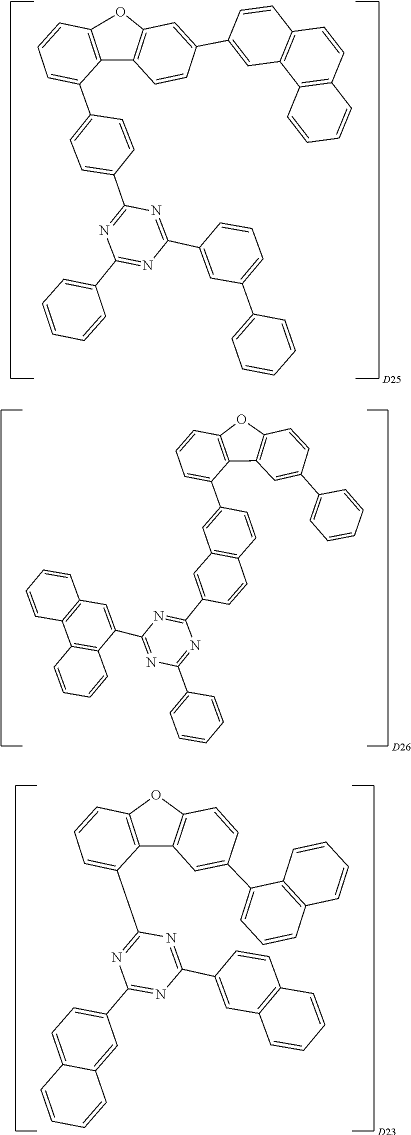

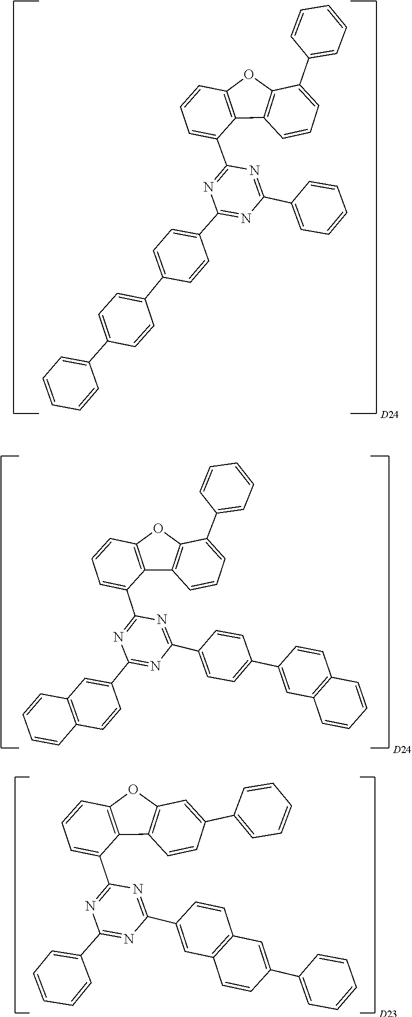

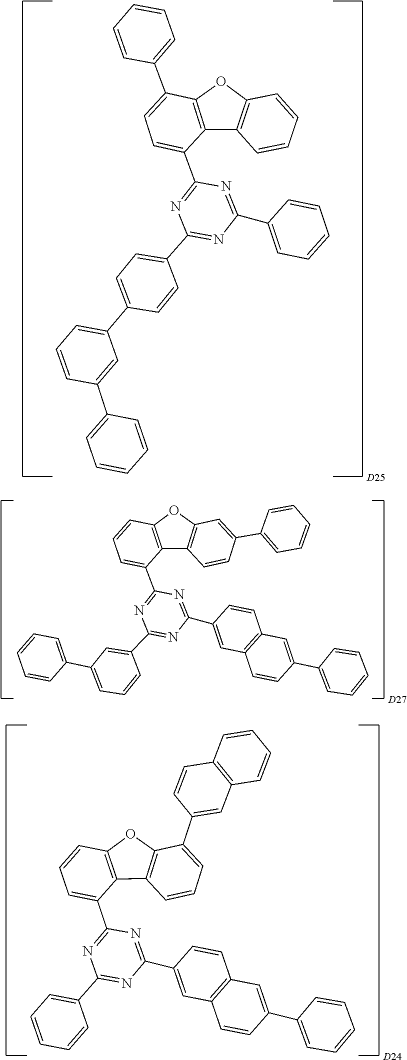

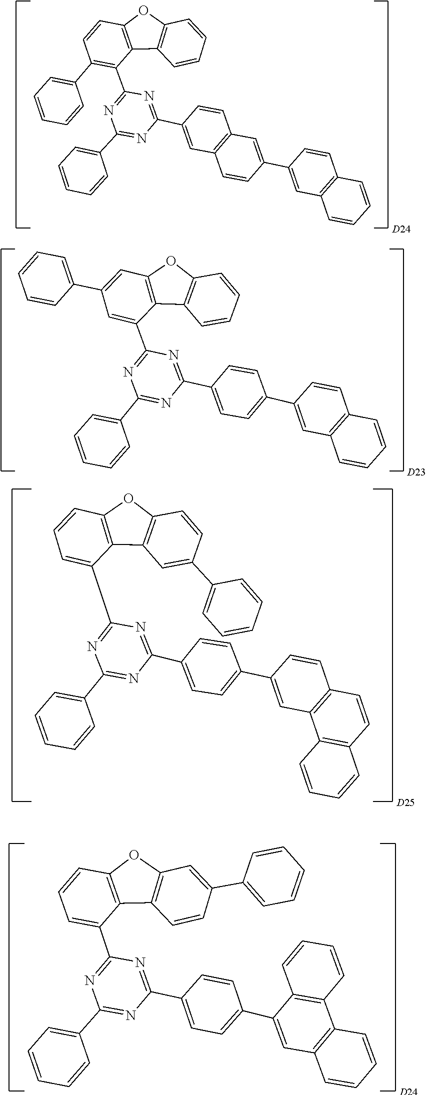

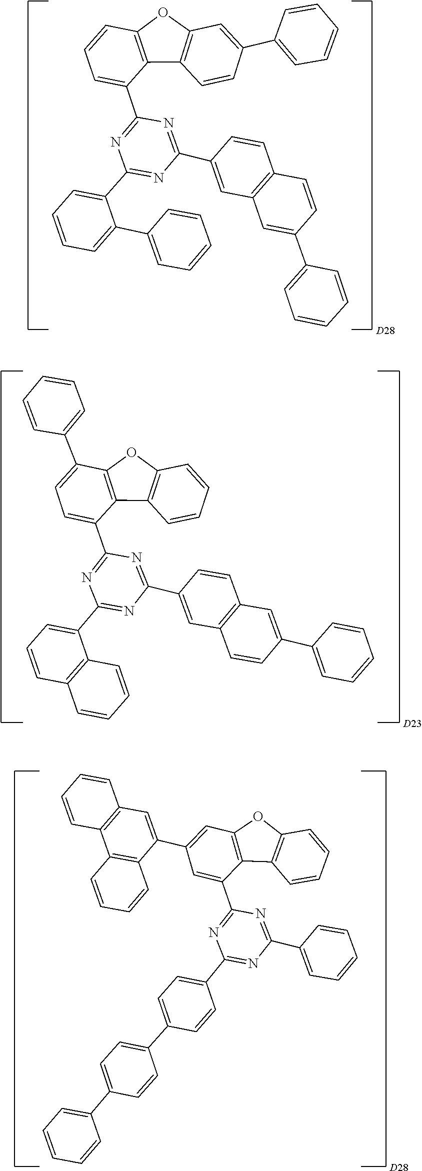

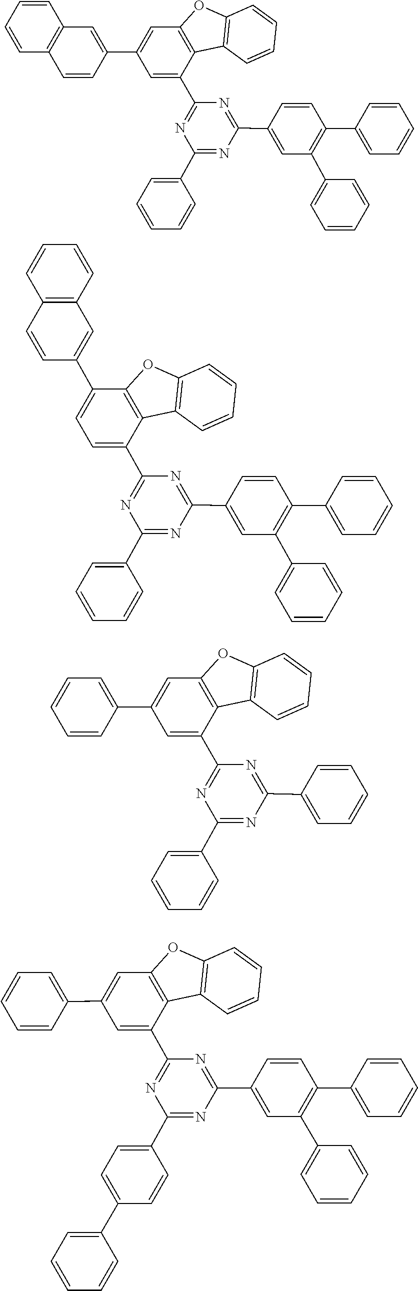

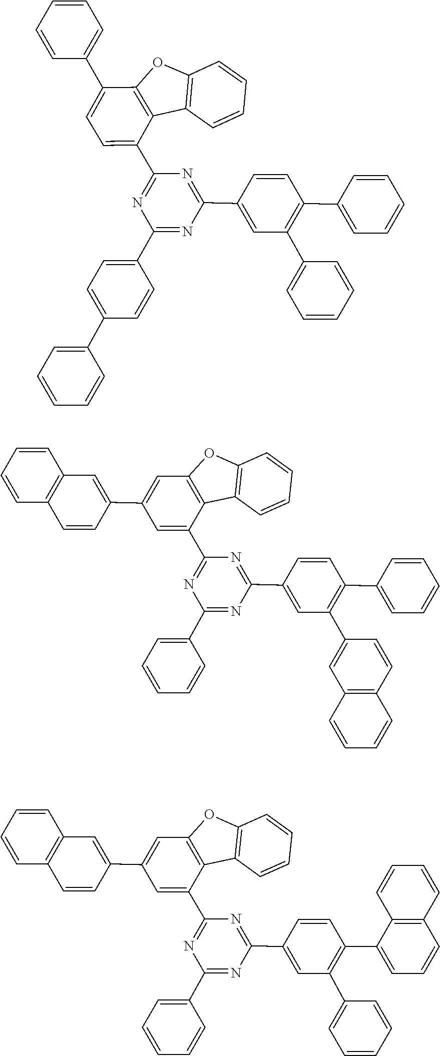

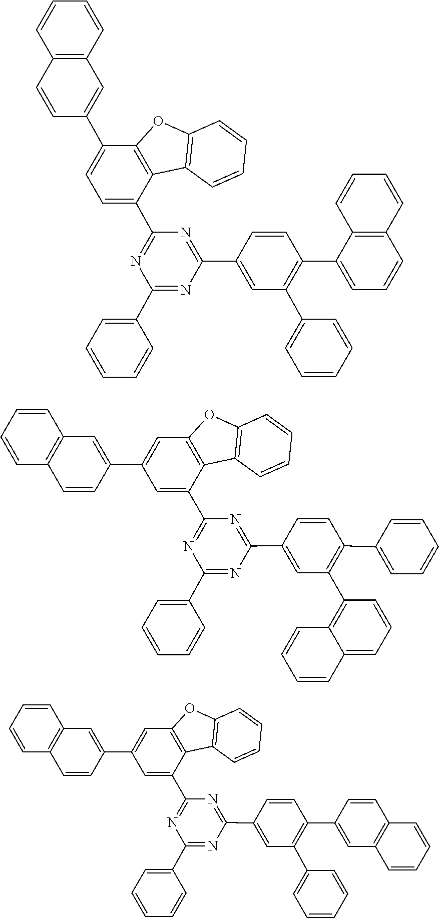 [SMILES: c1ccc(-c2nc(-c3ccc(-c4cccc5ccccc45)c(-c4ccccc4)c3)nc(-c3ccc(-c4ccc5ccccc5c4)c4oc5ccccc5c34)n2)cc1 ; c1ccc(-c2nc(-c3ccc(-c4ccccc4)c(-c4cccc5ccccc45)c3)nc(-c3cc(-c4ccc5ccccc5c4)cc4oc5ccccc5c34)n2)cc1 ; c1ccc(-c2nc(-c3ccc(-c4ccc5ccccc5c4)c(-c4ccccc4)c3)nc(-c3cc(-c4ccc5ccccc5c4)cc4oc5ccccc5c34)n2)cc1]

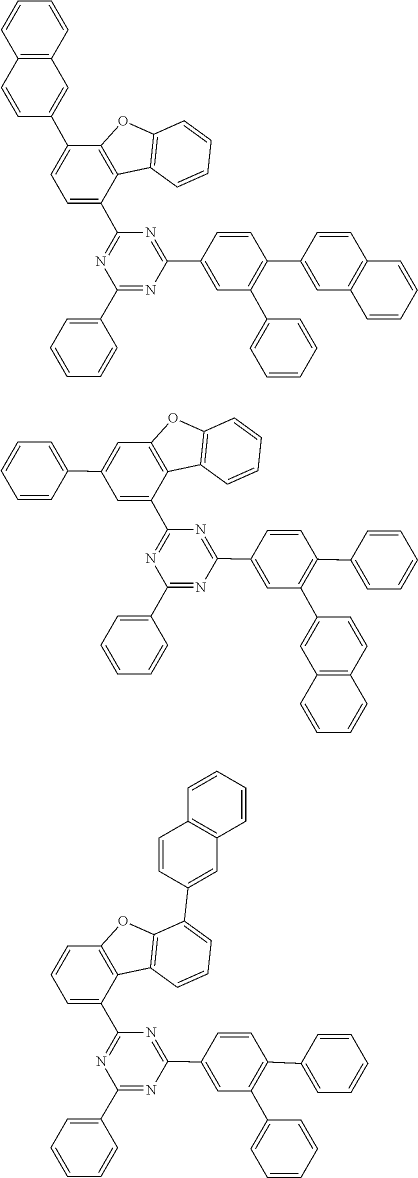 [SMILES: c1ccc(-c2nc(-c3ccc(-c4ccc5ccccc5c4)c(-c4ccccc4)c3)nc(-c3ccc(-c4ccc5ccccc5c4)c4oc5ccccc5c34)n2)cc1 ; c1ccc(-c2cc(-c3nc(-c4ccccc4)nc(-c4ccc(-c5ccccc5)c(-c5ccc6ccccc6c5)c4)n3)c3c(c2)oc2ccccc23)cc1 ; c1ccc(-c2nc(-c3ccc(-c4ccccc4)c(-c4ccccc4)c3)nc(-c3cccc4oc5c(-c6ccc7ccccc7c6)cccc5c34)n2)cc1]

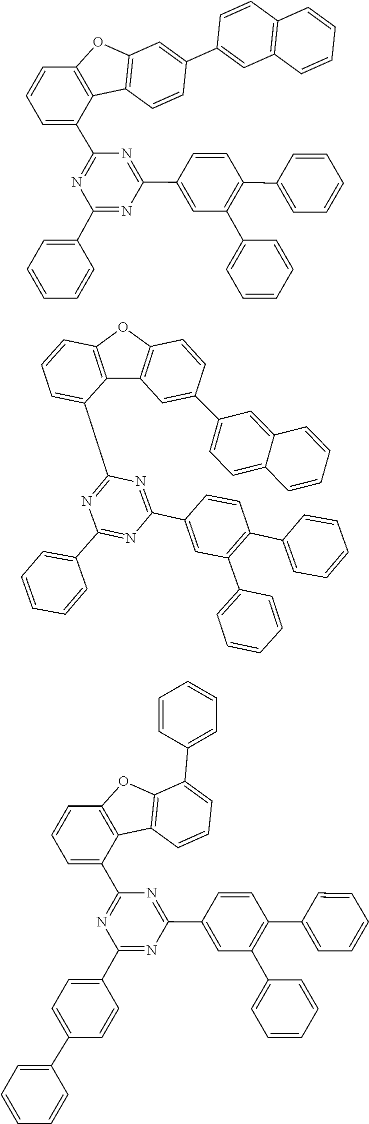

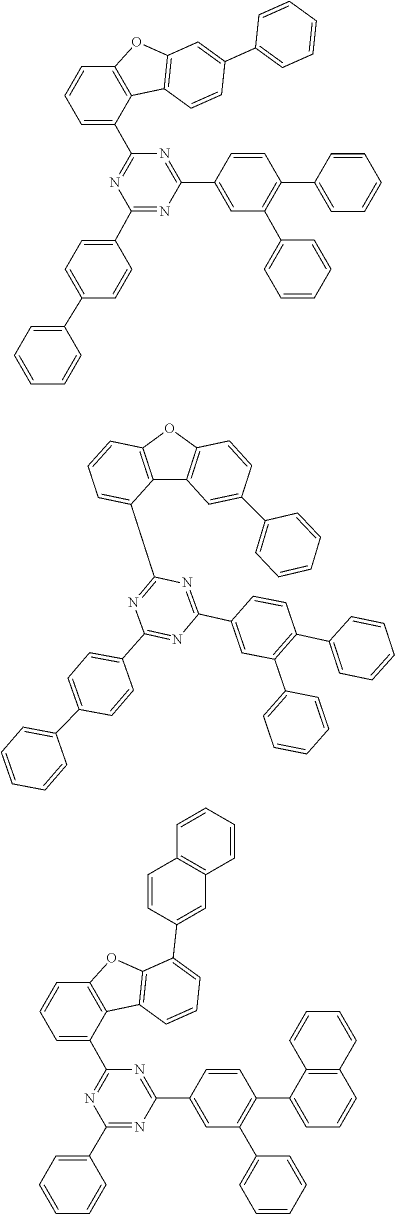 [SMILES: c1ccc(-c2ccc(-c3nc(-c4ccc(-c5ccccc5)c(-c5ccccc5)c4)nc(-c4cccc5oc6cc(-c7ccccc7)ccc6c45)n3)cc2)cc1 ; c1ccc(-c2ccc(-c3nc(-c4ccc(-c5ccccc5)c(-c5ccccc5)c4)nc(-c4cccc5oc6ccc(-c7ccccc7)cc6c45)n3)cc2)cc1 ; c1ccc(-c2nc(-c3ccc(-c4cccc5ccccc45)c(-c4ccccc4)c3)nc(-c3cccc4oc5c(-c6ccc7ccccc7c6)cccc5c34)n2)cc1]

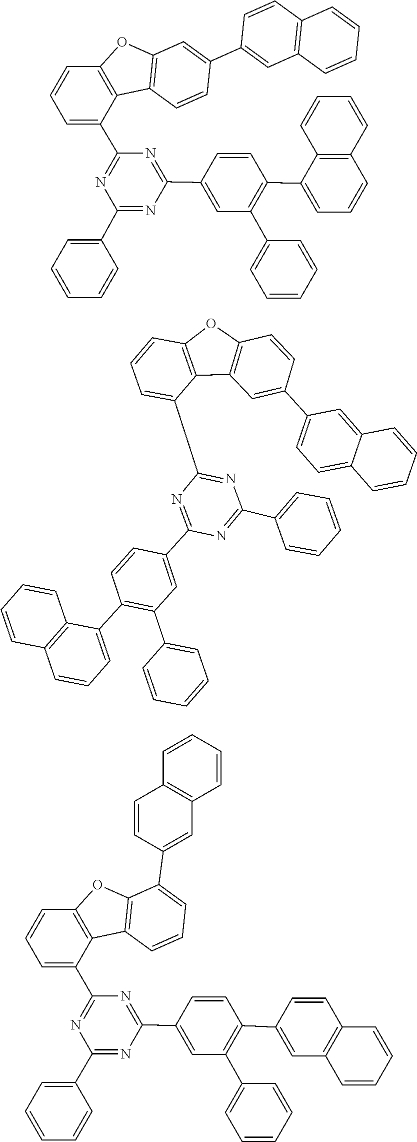

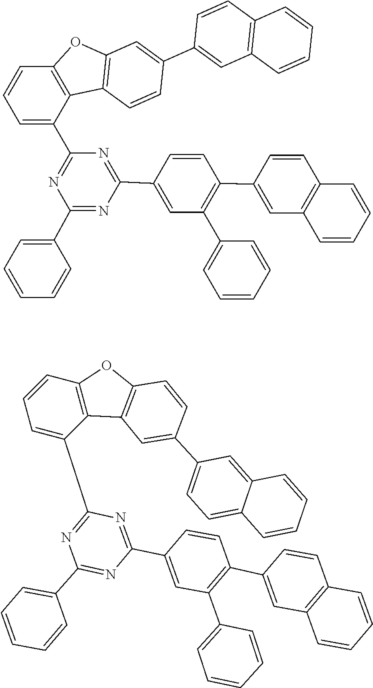 [SMILES: c1ccc(-c2nc(-c3ccc(-c4ccc5ccccc5c4)c(-c4ccccc4)c3)nc(-c3cccc4oc5cc(-c6ccc7ccccc7c6)ccc5c34)n2)cc1 ; c1ccc(-c2nc(-c3ccc(-c4ccc5ccccc5c4)c(-c4ccccc4)c3)nc(-c3cccc4oc5ccc(-c6ccc7ccccc7c6)cc5c34)n2)cc1]

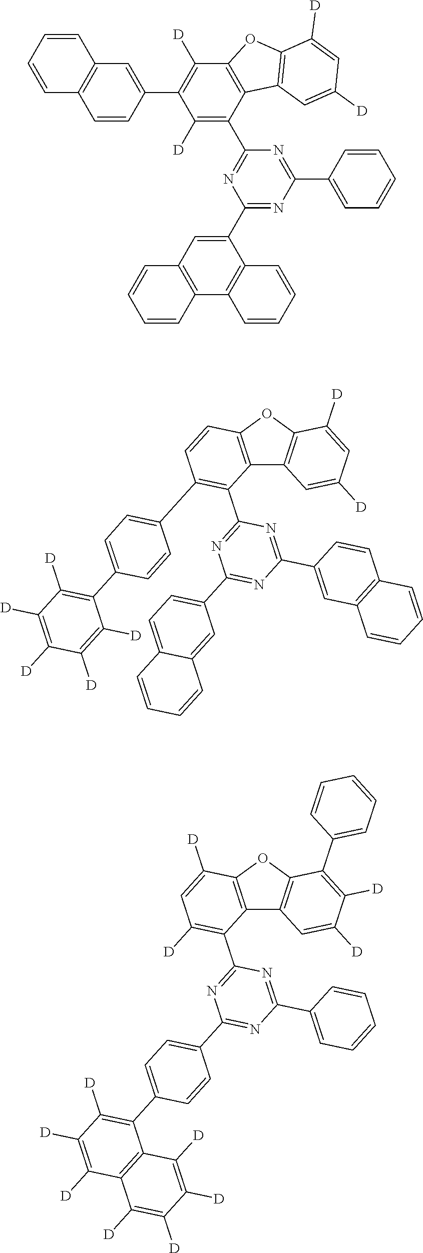 [SMILES: [2H]c1cc([2H])c2oc3c([2H])c(-c4ccc5ccccc5c4)c([2H])c(-c4nc(-c5ccccc5)nc(-c5cc6ccccc6c6ccccc56)n4)c3c2c1 ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H]]

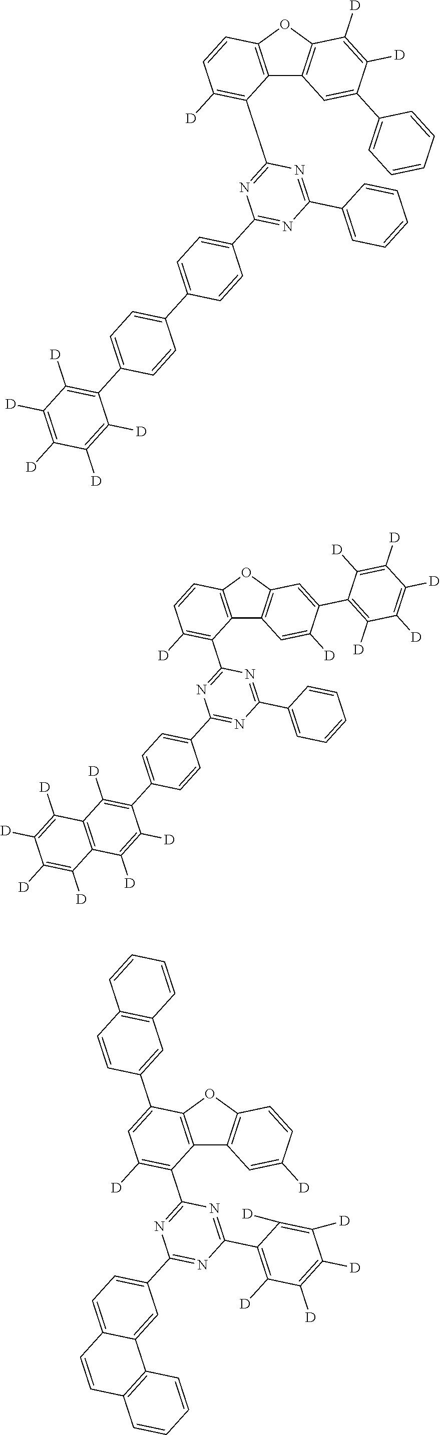 [SMILES: [2H]c1ccc2oc3c([2H])c([2H])c(-c4ccccc4)cc3c2c1-c1nc(-c2ccccc2)nc(-c2ccc(-c3ccc(-c4c([2H])c([2H])c([2H])c([2H])c4[2H])cc3)cc2)n1 ; [2H]c1cc2c(cc1-c1c([2H])c([2H])c([2H])c([2H])c1[2H])oc1ccc([2H])c(-c3nc(-c4ccccc4)nc(-c4ccc(-c5c([2H])c([2H])c6c([2H])c([2H])c([2H])c([2H])c6c5[2H])cc4)n3)c12 ; [2H]c1ccc2oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c3c2c1]

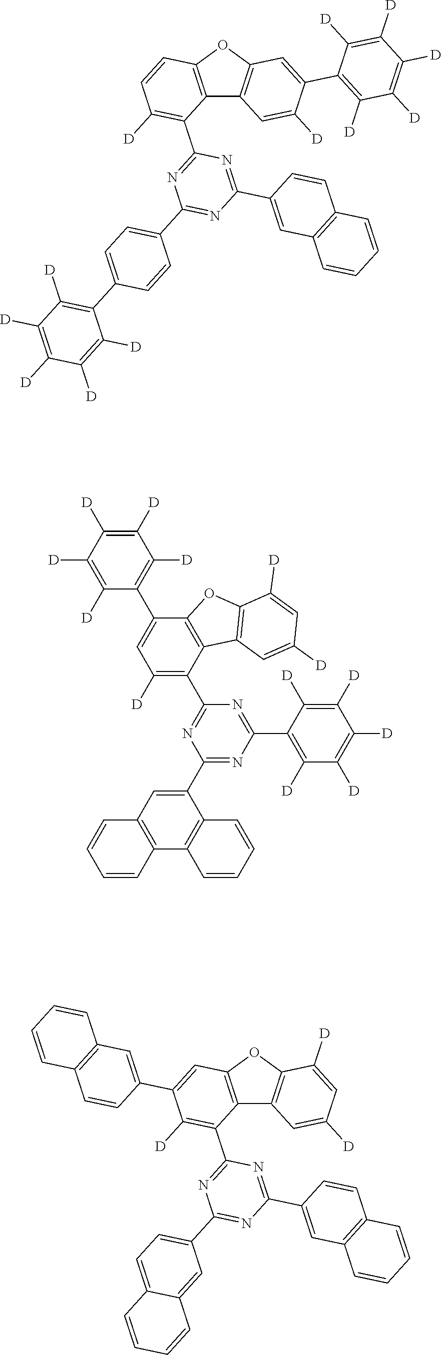 [SMILES: [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12 ; [2H]c1cc([2H])c2oc3c(-c4c([2H])c([2H])c([2H])c([2H])c4[2H])cc([2H])c(-c4nc(-c5c([2H])c([2H])c([2H])c([2H])c5[2H])nc(-c5cc6ccccc6c6ccccc56)n4)c3c2c1 ; [2H]c1cc([2H])c2oc3cc(-c4ccc5ccccc5c4)c([2H])c(-c4nc(-c5ccc6ccccc6c5)nc(-c5ccc6ccccc6c5)n4)c3c2c1]

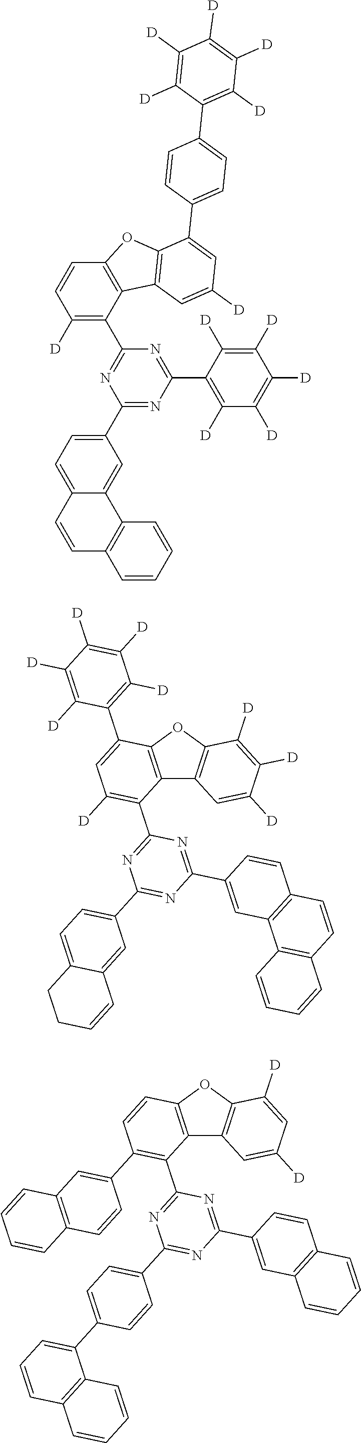 [SMILES: [2H]c1cc(-c2ccc(-c3c([2H])c([2H])c([2H])c([2H])c3[2H])cc2)c2oc3ccc([2H])c(-c4nc(-c5ccc6ccc7ccccc7c6c5)nc(-c5c([2H])c([2H])c([2H])c([2H])c5[2H])n4)c3c2c1 ; [2H]c1cc2c(oc3c(-c4c([2H])c([2H])c([2H])c([2H])c4[2H])cc([2H])c(-c4nc(-c5ccc6c(c5)C=CCC6)nc(-c5ccc6ccc7ccccc7c6c5)n4)c32)c([2H])c1[2H] ; [2H]c1cc([2H])c2oc3ccc(-c4ccc5ccccc5c4)c(-c4nc(-c5ccc(-c6cccc7ccccc67)cc5)nc(-c5ccc6ccccc6c5)n4)c3c2c1]

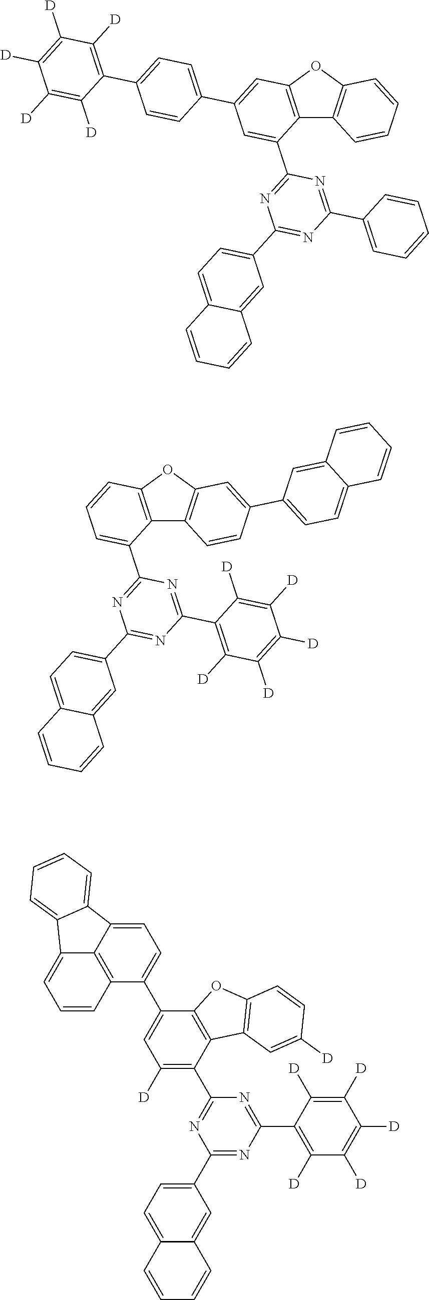 [SMILES: [2H]c1c([2H])c([2H])c(-c2ccc(-c3cc(-c4nc(-c5ccccc5)nc(-c5ccc6ccccc6c5)n4)c4c(c3)oc3ccccc34)cc2)c([2H])c1[2H] ; [2H]c1c([2H])c([2H])c(-c2nc(-c3ccc4ccccc4c3)nc(-c3cccc4oc5cc(-c6ccc7ccccc7c6)ccc5c34)n2)c([2H])c1[2H] ; [2H]c1ccc2oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c3c2c1]

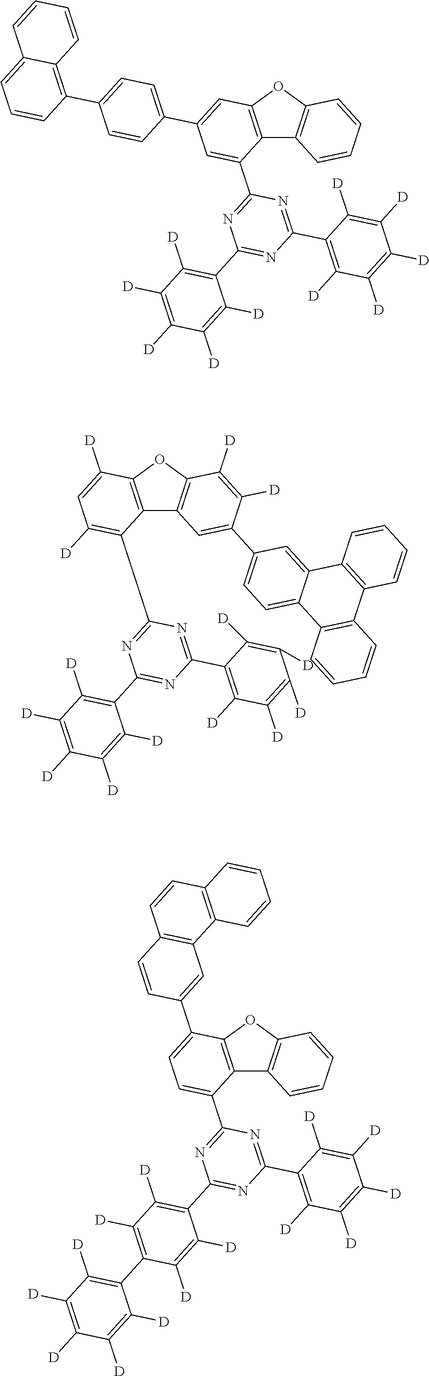 [SMILES: [2H]c1c([2H])c([2H])c(-c2nc(-c3c([2H])c([2H])c([2H])c([2H])c3[2H])nc(-c3cc(-c4ccc(-c5cccc6ccccc56)cc4)cc4oc5ccccc5c34)n2)c([2H])c1[2H] ; [2H]c1cc([2H])c2oc3c([2H])c([2H])c(-c4ccc5c6ccccc6c6ccccc6c5c4)cc3c2c1-c1nc(-c2c([2H])c([2H])c([2H])c([2H])c2[2H])nc(-c2c([2H])c([2H])c([2H])c([2H])c2[2H])n1 ; [2H]c1c([2H])c([2H])c(-c2nc(-c3c([2H])c([2H])c(-c4c([2H])c([2H])c([2H])c([2H])c4[2H])c([2H])c3[2H])nc(-c3ccc(-c4ccc5ccc6ccccc6c5c4)c4oc5ccccc5c34)n2)c([2H])c1[2H]]

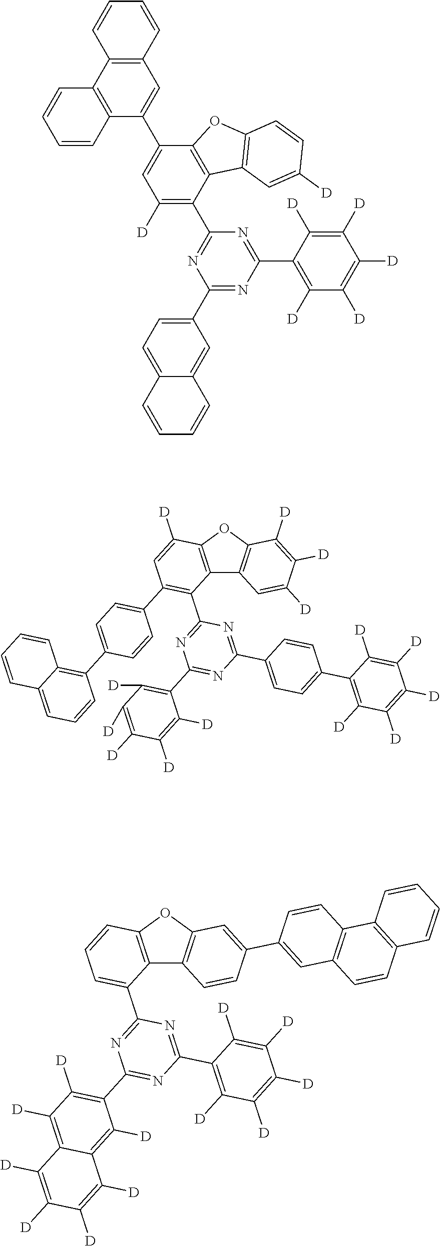 [SMILES: [2H]c1ccc2oc3c(-c4cc5ccccc5c5ccccc45)cc([2H])c(-c4nc(-c5ccc6ccccc6c5)nc(-c5c([2H])c([2H])c([2H])c([2H])c5[2H])n4)c3c2c1 ; [2H]c1cc2c(oc3c([2H])cc(-c4ccc(-c5cccc6ccccc56)cc4)c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H]]

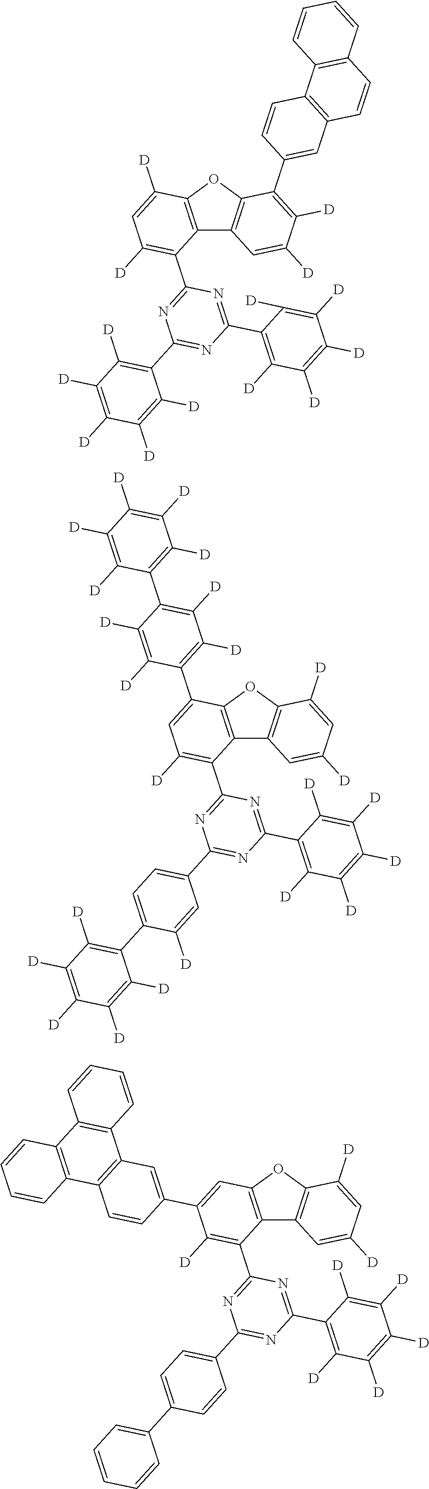

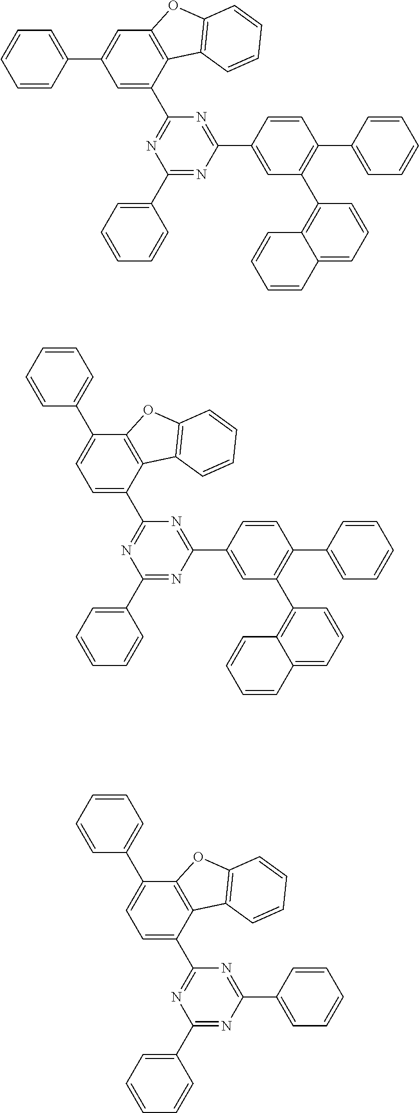 [SMILES: c1ccc(-c2cc(-c3nc(-c4ccccc4)nc(-c4ccc(-c5ccccc5)c(-c5cccc6ccccc56)c4)n3)c3c(c2)oc2ccccc23)cc1 ; c1ccc(-c2nc(-c3ccc(-c4ccccc4)c(-c4cccc5ccccc45)c3)nc(-c3ccc(-c4ccccc4)c4oc5ccccc5c34)n2)cc1 ; c1ccc(-c2nc(-c3ccccc3)nc(-c3ccc(-c4ccccc4)c4oc5ccccc5c34)n2)cc1]

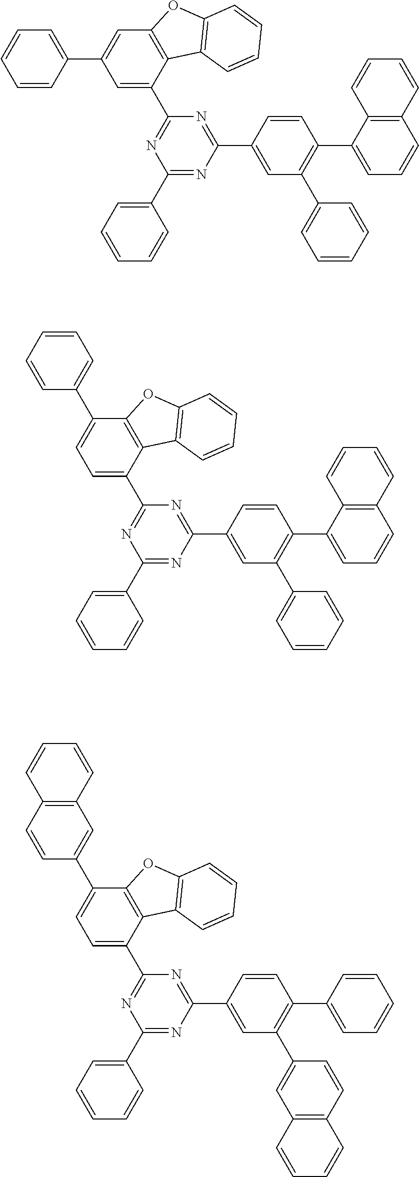 [SMILES: c1ccc(-c2cc(-c3nc(-c4ccccc4)nc(-c4ccc(-c5cccc6ccccc56)c(-c5ccccc5)c4)n3)c3c(c2)oc2ccccc23)cc1 ; c1ccc(-c2nc(-c3ccc(-c4cccc5ccccc45)c(-c4ccccc4)c3)nc(-c3ccc(-c4ccccc4)c4oc5ccccc5c34)n2)cc1 ; c1ccc(-c2nc(-c3ccc(-c4ccccc4)c(-c4ccc5ccccc5c4)c3)nc(-c3ccc(-c4ccc5ccccc5c4)c4oc5ccccc5c34)n2)cc1]

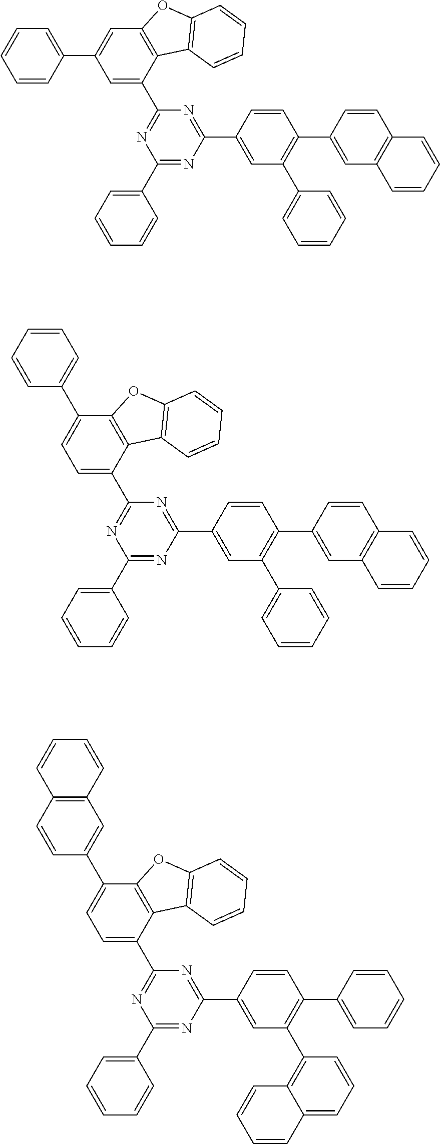 [SMILES: c1ccc(-c2cc(-c3nc(-c4ccccc4)nc(-c4ccc(-c5ccc6ccccc6c5)c(-c5ccccc5)c4)n3)c3c(c2)oc2ccccc23)cc1 ; c1ccc(-c2nc(-c3ccc(-c4ccc5ccccc5c4)c(-c4ccccc4)c3)nc(-c3ccc(-c4ccccc4)c4oc5ccccc5c34)n2)cc1 ; c1ccc(-c2nc(-c3ccc(-c4ccccc4)c(-c4cccc5ccccc45)c3)nc(-c3ccc(-c4ccc5ccccc5c4)c4oc5ccccc5c34)n2)cc1]

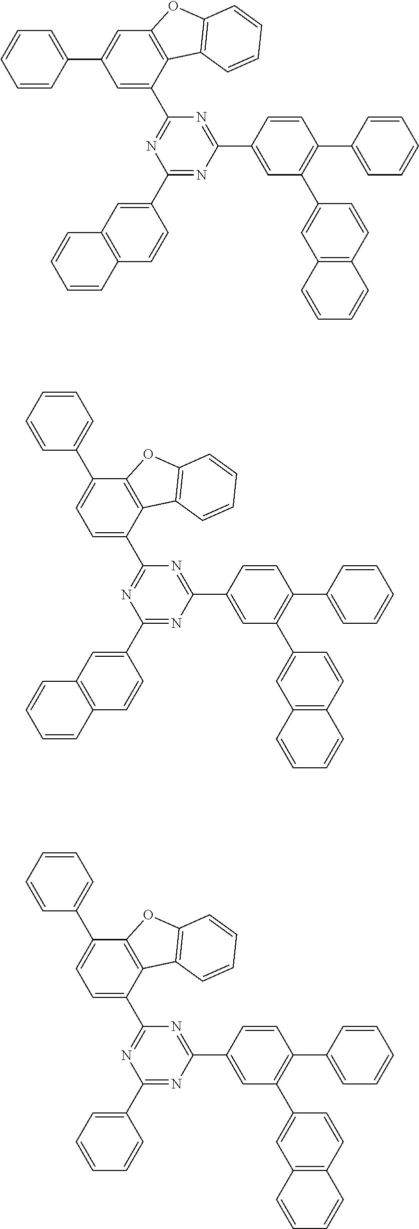 [SMILES: c1ccc(-c2cc(-c3nc(-c4ccc(-c5ccccc5)c(-c5ccc6ccccc6c5)c4)nc(-c4ccc5ccccc5c4)n3)c3c(c2)oc2ccccc23)cc1 ; c1ccc(-c2ccc(-c3nc(-c4ccc5ccccc5c4)nc(-c4ccc(-c5ccccc5)c5oc6ccccc6c45)n3)cc2-c2ccc3ccccc3c2)cc1 ; c1ccc(-c2nc(-c3ccc(-c4ccccc4)c(-c4ccc5ccccc5c4)c3)nc(-c3ccc(-c4ccccc4)c4oc5ccccc5c34)n2)cc1]

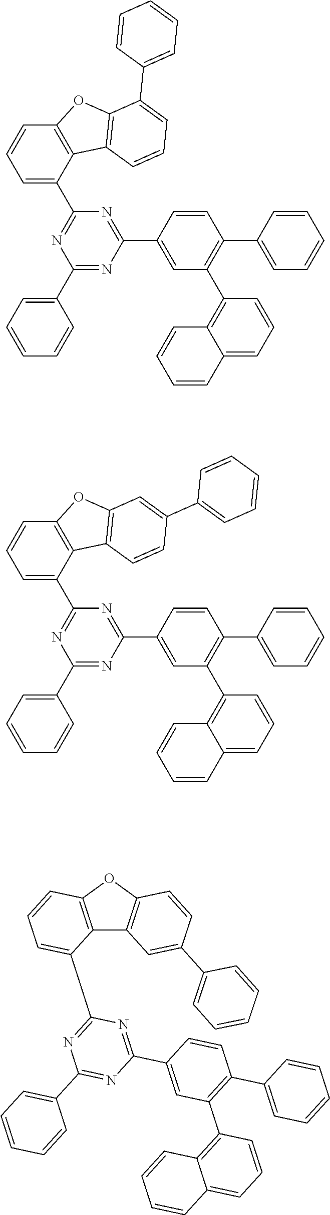 [SMILES: c1ccc(-c2nc(-c3ccc(-c4ccccc4)c(-c4cccc5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccccc6)c(-c6cccc7ccccc67)c5)n4)c23)cc1 ; c1ccc(-c2ccc3oc4cccc(-c5nc(-c6ccccc6)nc(-c6ccc(-c7ccccc7)c(-c7cccc8ccccc78)c6)n5)c4c3c2)cc1]

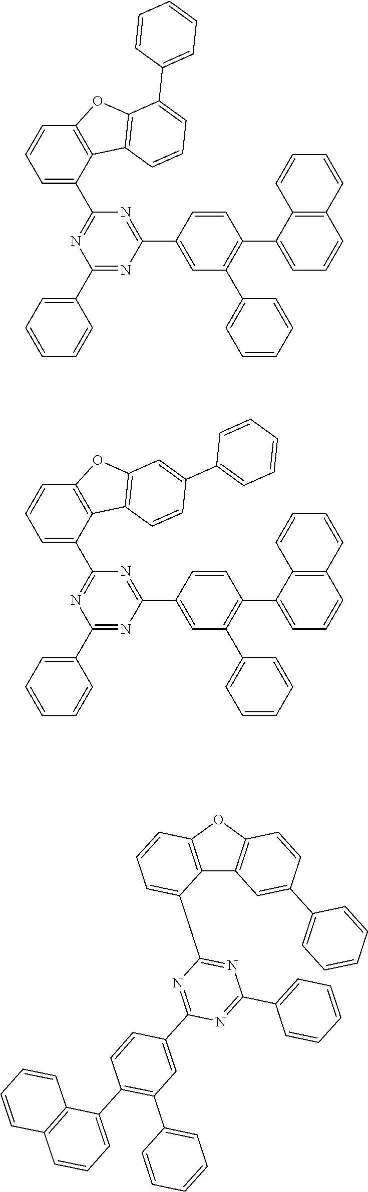 [SMILES: c1ccc(-c2nc(-c3ccc(-c4cccc5ccccc45)c(-c4ccccc4)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6cccc7ccccc67)c(-c6ccccc6)c5)n4)c23)cc1 ; c1ccc(-c2ccc3oc4cccc(-c5nc(-c6ccccc6)nc(-c6ccc(-c7cccc8ccccc78)c(-c7ccccc7)c6)n5)c4c3c2)cc1]

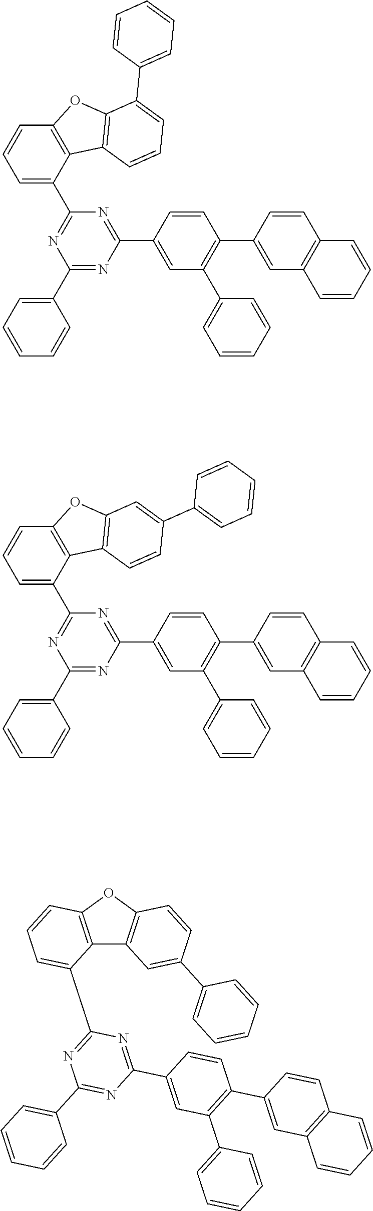 [SMILES: c1ccc(-c2nc(-c3ccc(-c4ccc5ccccc5c4)c(-c4ccccc4)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccc7ccccc7c6)c(-c6ccccc6)c5)n4)c23)cc1 ; c1ccc(-c2ccc3oc4cccc(-c5nc(-c6ccccc6)nc(-c6ccc(-c7ccc8ccccc8c7)c(-c7ccccc7)c6)n5)c4c3c2)cc1]

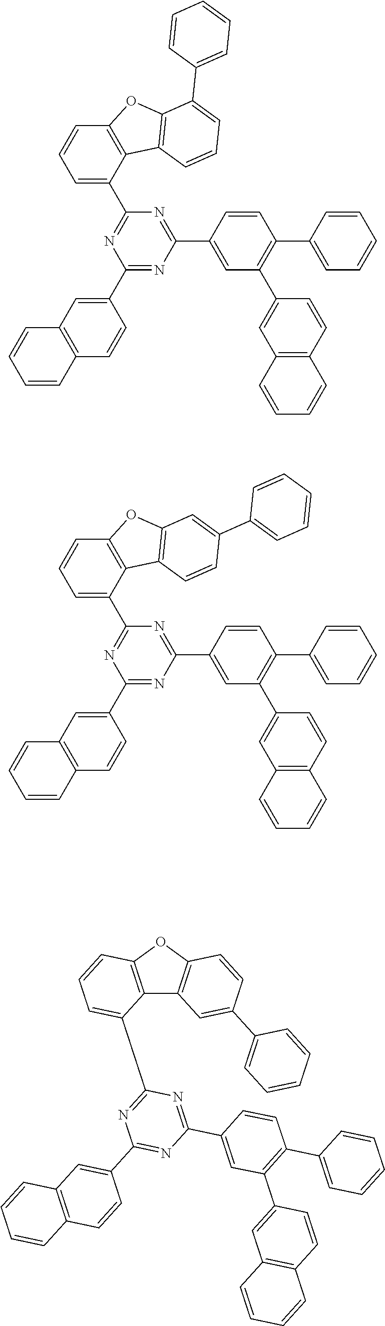 [SMILES: c1ccc(-c2ccc(-c3nc(-c4ccc5ccccc5c4)nc(-c4cccc5oc6c(-c7ccccc7)cccc6c45)n3)cc2-c2ccc3ccccc3c2)cc1 ; c1ccc(-c2ccc3c(c2)oc2cccc(-c4nc(-c5ccc(-c6ccccc6)c(-c6ccc7ccccc7c6)c5)nc(-c5ccc6ccccc6c5)n4)c23)cc1 ; c1ccc(-c2ccc3oc4cccc(-c5nc(-c6ccc(-c7ccccc7)c(-c7ccc8ccccc8c7)c6)nc(-c6ccc7ccccc7c6)n5)c4c3c2)cc1]

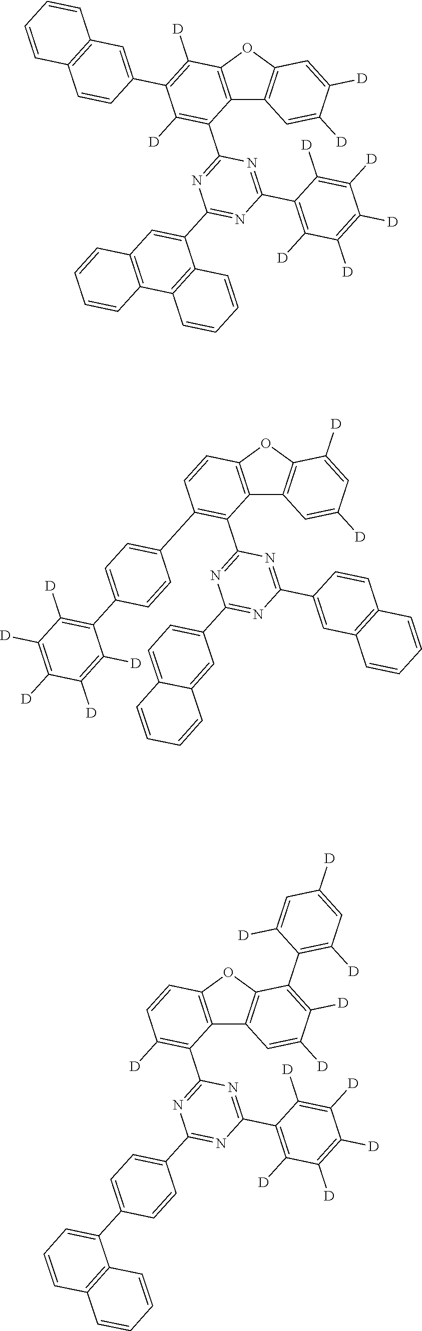 [SMILES: [2H]c1cc2oc3c([2H])c(-c4ccc5ccccc5c4)c([2H])c(-c4nc(-c5c([2H])c([2H])c([2H])c([2H])c5[2H])nc(-c5cc6ccccc6c6ccccc56)n4)c3c2cc1[2H] ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc([2H])c(-c2c([2H])c([2H])cc3c2oc2ccc([2H])c(-c4nc(-c5ccc(-c6cccc7ccccc67)cc5)nc(-c5c([2H])c([2H])c([2H])c([2H])c5[2H])n4)c23)c([2H])c1]

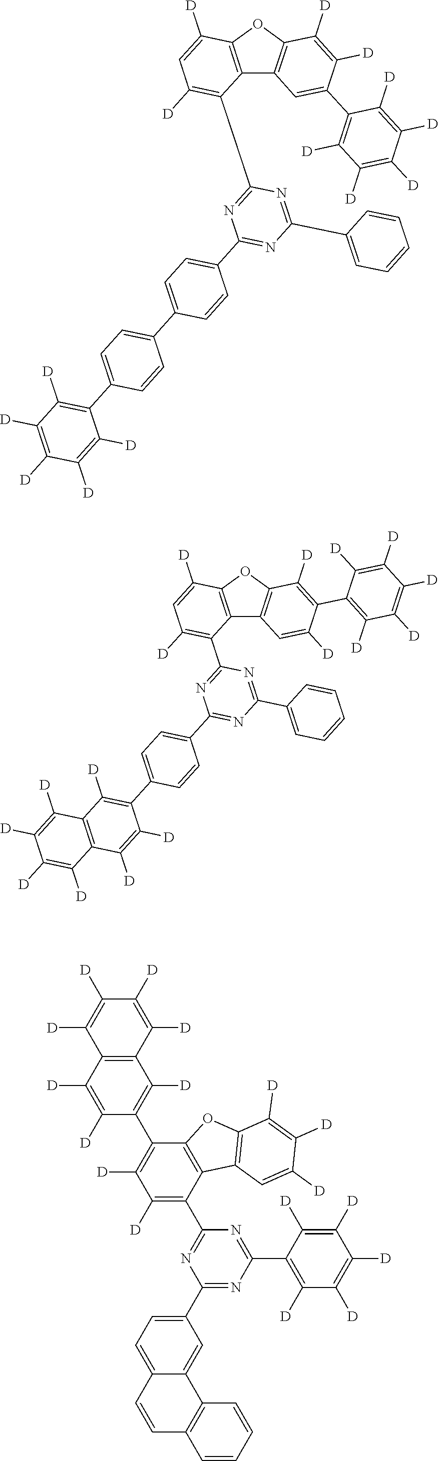

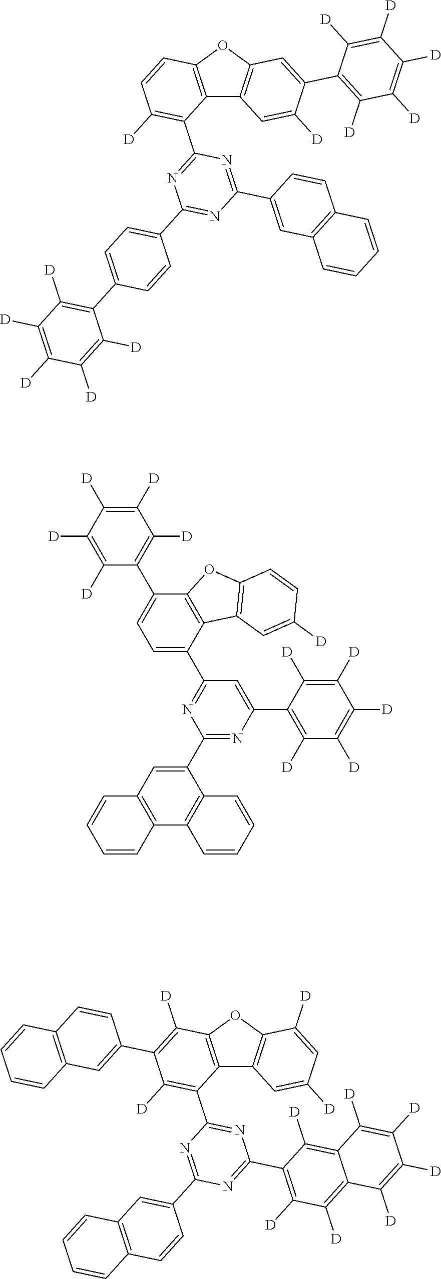 [SMILES: [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12 ; [2H]c1ccc2oc3c(-c4c([2H])c([2H])c([2H])c([2H])c4[2H])ccc(-c4cc(-c5c([2H])c([2H])c([2H])c([2H])c5[2H])nc(-c5cc6ccccc6c6ccccc56)n4)c3c2c1 ; [2H]c1cc([2H])c2oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c3c2c1]

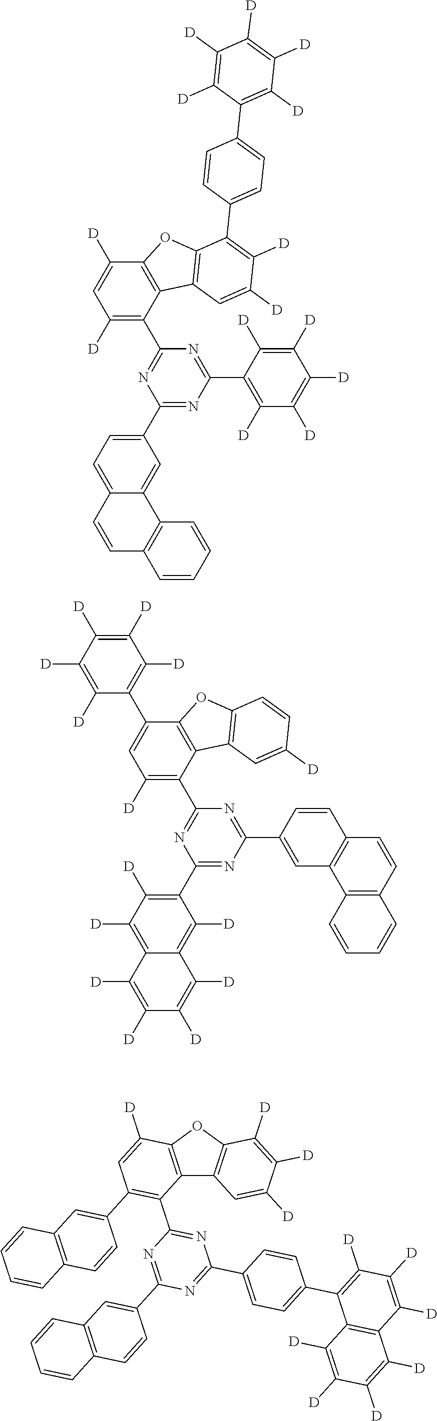

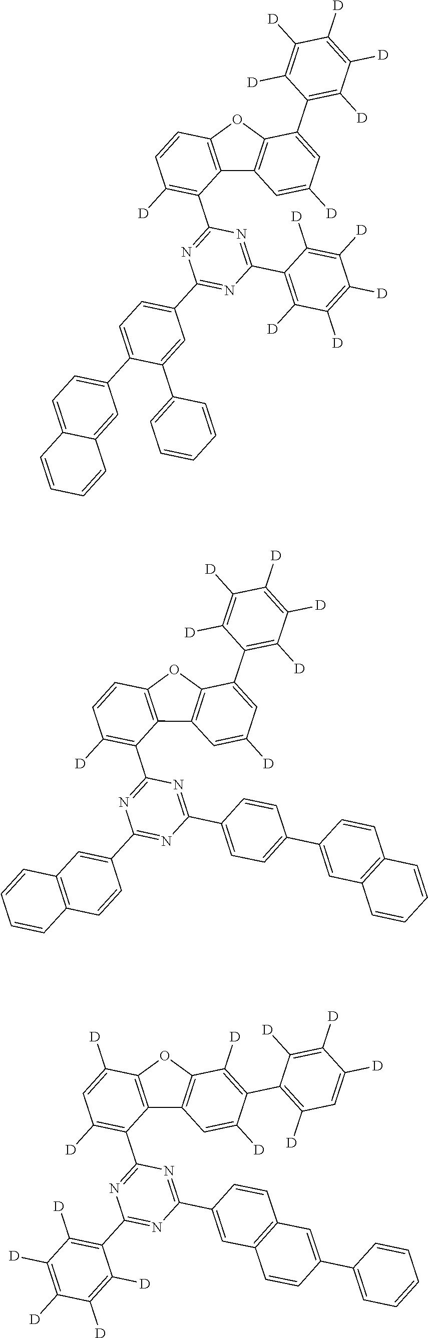 [SMILES: [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1 ; [2H]c1cc([2H])c(-c2c([2H])cc3c(oc4c([2H])cc([2H])c(-c5nc(-c6ccc7cc(-c8ccccc8)ccc7c6)nc(-c6c([2H])c([2H])c([2H])c([2H])c6[2H])n5)c43)c2[2H])c([2H])c1[2H]]

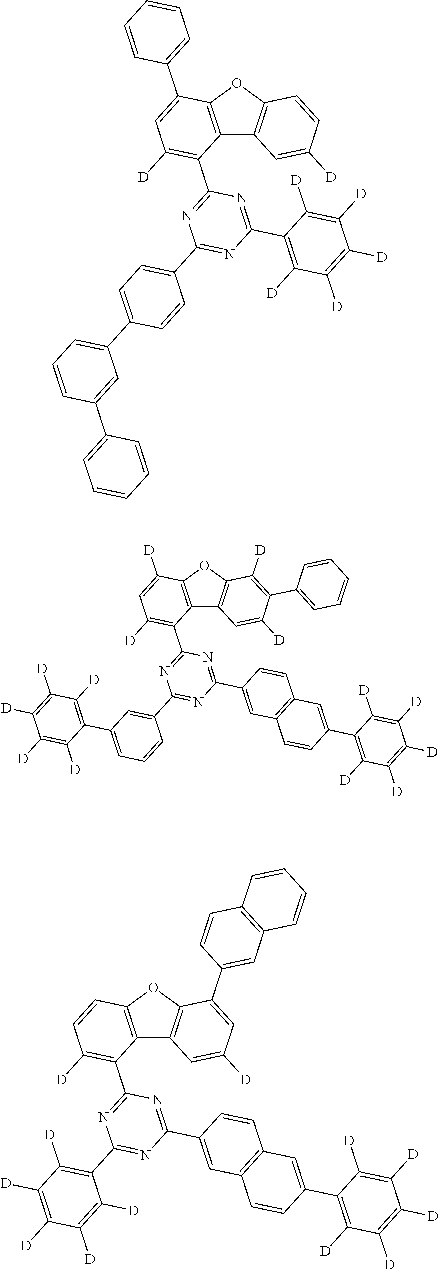 [SMILES: [2H]c1ccc2oc3c(-c4ccccc4)cc([2H])c(-c4nc(-c5ccc(-c6cccc(-c7ccccc7)c6)cc5)nc(-c5c([2H])c([2H])c([2H])c([2H])c5[2H])n4)c3c2c1 ; [2H]c1cc2c(oc3c([2H])cc([2H])c(-c4nc(-c5cccc(-c6c([2H])c([2H])c([2H])c([2H])c6[2H])c5)nc(-c5ccc6cc(-c7c([2H])c([2H])c([2H])c([2H])c7[2H])ccc6c5)n4)c32)c([2H])c1-c1ccccc1 ; [2H]c1cc(-c2ccc3ccccc3c2)c2oc3ccc([2H])c(-c4nc(-c5ccc6cc(-c7c([2H])c([2H])c([2H])c([2H])c7[2H])ccc6c5)nc(-c5c([2H])c([2H])c([2H])c([2H])c5[2H])n4)c3c2c1]

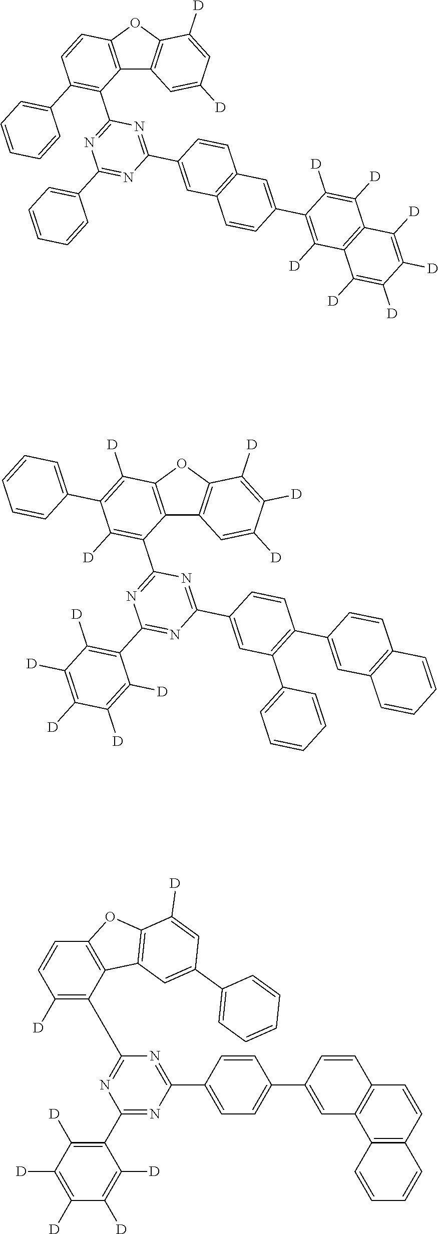 [SMILES: [2H]c1cc([2H])c2oc3ccc(-c4ccccc4)c(-c4nc(-c5ccccc5)nc(-c5ccc6cc(-c7c([2H])c([2H])c8c([2H])c([2H])c([2H])c([2H])c8c7[2H])ccc6c5)n4)c3c2c1 ; [2H]c1cc2c(oc3c([2H])c(-c4ccccc4)c([2H])c(-c4nc(-c5ccc(-c6ccc7ccccc7c6)c(-c6ccccc6)c5)nc(-c5c([2H])c([2H])c([2H])c([2H])c5[2H])n4)c32)c([2H])c1[2H] ; [2H]c1ccc2oc3c([2H])cc(-c4ccccc4)cc3c2c1-c1nc(-c2ccc(-c3ccc4ccc5ccccc5c4c3)cc2)nc(-c2c([2H])c([2H])c([2H])c([2H])c2[2H])n1]

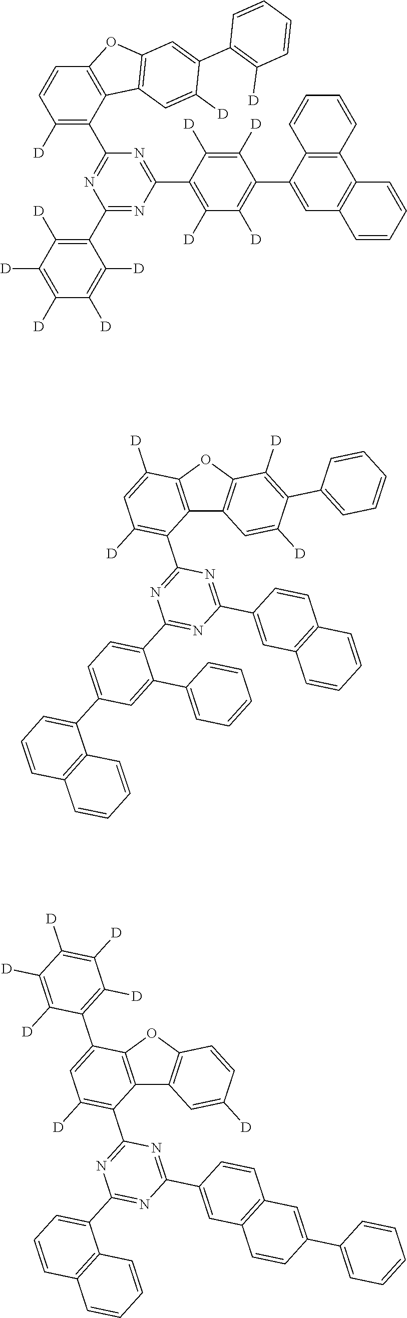 [SMILES: [2H]c1ccccc1-c1cc2oc3ccc([2H])c(-c4nc(-c5c([2H])c([2H])c([2H])c([2H])c5[2H])nc(-c5c([2H])c([2H])c(-c6cc7ccccc7c7ccccc67)c([2H])c5[2H])n4)c3c2cc1[2H] ; [2H]c1cc2c(oc3c([2H])cc([2H])c(-c4nc(-c5ccc6ccccc6c5)nc(-c5ccc(-c6cccc7ccccc67)cc5-c5ccccc5)n4)c32)c([2H])c1-c1ccccc1 ; [2H]c1ccc2oc3c(-c4c([2H])c([2H])c([2H])c([2H])c4[2H])cc([2H])c(-c4nc(-c5ccc6cc(-c7ccccc7)ccc6c5)nc(-c5cccc6ccccc56)n4)c3c2c1]

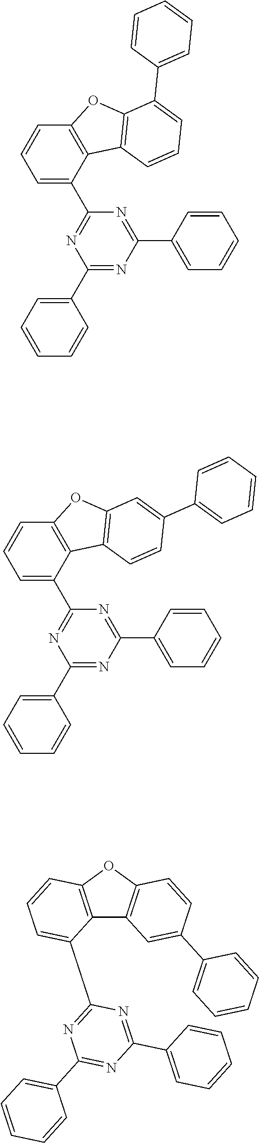 [SMILES: c1ccc(-c2nc(-c3ccccc3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccccc5)n4)c23)cc1 ; c1ccc(-c2ccc3oc4cccc(-c5nc(-c6ccccc6)nc(-c6ccccc6)n5)c4c3c2)cc1]

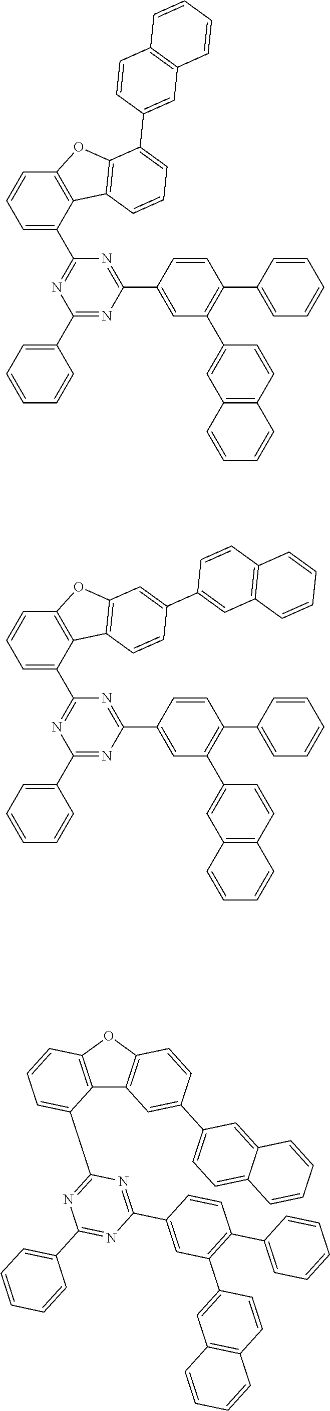 [SMILES: c1ccc(-c2nc(-c3ccc(-c4ccccc4)c(-c4ccc5ccccc5c4)c3)nc(-c3cccc4oc5c(-c6ccc7ccccc7c6)cccc5c34)n2)cc1 ; c1ccc(-c2nc(-c3ccc(-c4ccccc4)c(-c4ccc5ccccc5c4)c3)nc(-c3cccc4oc5cc(-c6ccc7ccccc7c6)ccc5c34)n2)cc1 ; c1ccc(-c2nc(-c3ccc(-c4ccccc4)c(-c4ccc5ccccc5c4)c3)nc(-c3cccc4oc5ccc(-c6ccc7ccccc7c6)cc5c34)n2)cc1]

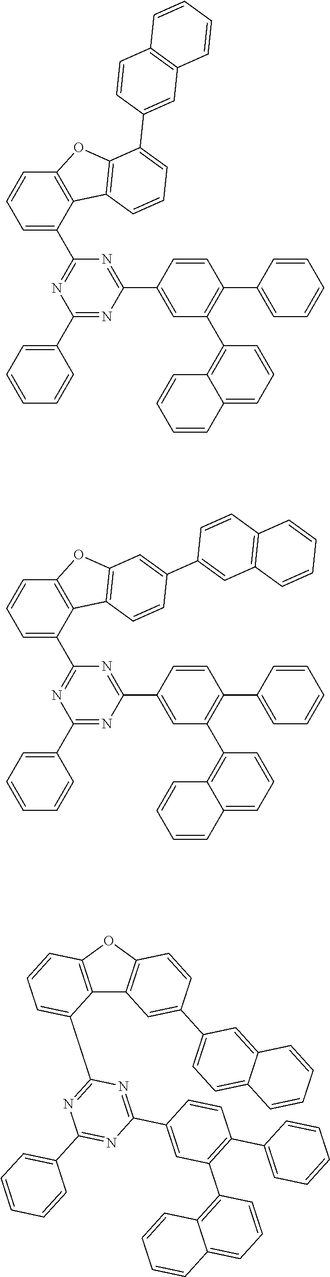 [SMILES: c1ccc(-c2nc(-c3ccc(-c4ccccc4)c(-c4cccc5ccccc45)c3)nc(-c3cccc4oc5c(-c6ccc7ccccc7c6)cccc5c34)n2)cc1 ; c1ccc(-c2nc(-c3ccc(-c4ccccc4)c(-c4cccc5ccccc45)c3)nc(-c3cccc4oc5cc(-c6ccc7ccccc7c6)ccc5c34)n2)cc1 ; c1ccc(-c2nc(-c3ccc(-c4ccccc4)c(-c4cccc5ccccc45)c3)nc(-c3cccc4oc5ccc(-c6ccc7ccccc7c6)cc5c34)n2)cc1]

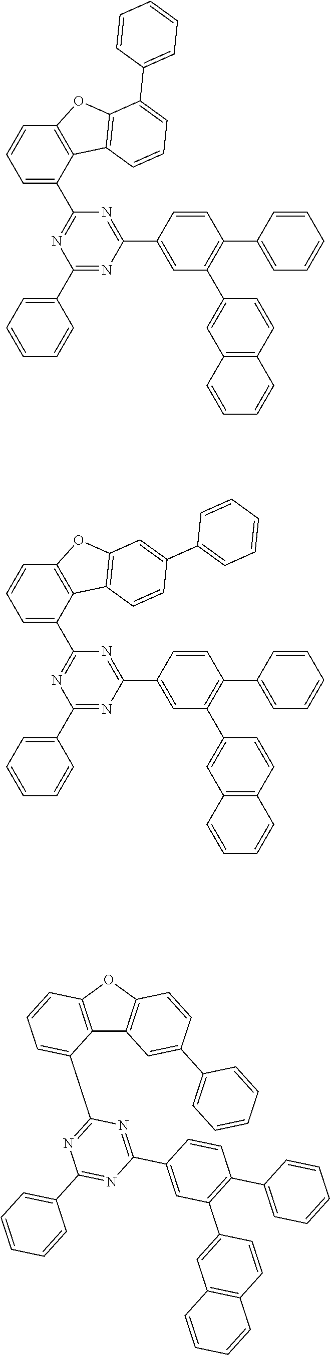 [SMILES: c1ccc(-c2nc(-c3ccc(-c4ccccc4)c(-c4ccc5ccccc5c4)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccccc6)c(-c6ccc7ccccc7c6)c5)n4)c23)cc1 ; c1ccc(-c2ccc3oc4cccc(-c5nc(-c6ccccc6)nc(-c6ccc(-c7ccccc7)c(-c7ccc8ccccc8c7)c6)n5)c4c3c2)cc1]

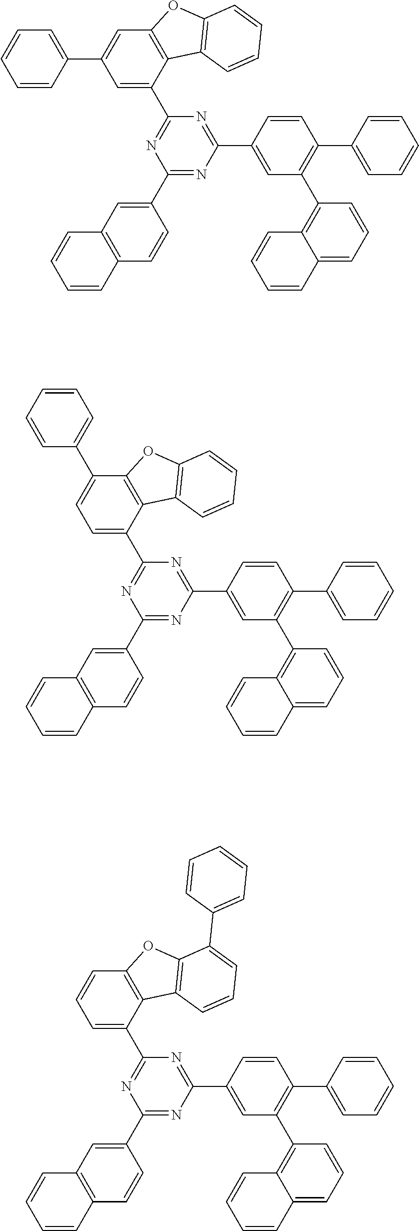 [SMILES: c1ccc(-c2cc(-c3nc(-c4ccc(-c5ccccc5)c(-c5cccc6ccccc56)c4)nc(-c4ccc5ccccc5c4)n3)c3c(c2)oc2ccccc23)cc1 ; c1ccc(-c2ccc(-c3nc(-c4ccc5ccccc5c4)nc(-c4ccc(-c5ccccc5)c5oc6ccccc6c45)n3)cc2-c2cccc3ccccc23)cc1 ; c1ccc(-c2ccc(-c3nc(-c4ccc5ccccc5c4)nc(-c4cccc5oc6c(-c7ccccc7)cccc6c45)n3)cc2-c2cccc3ccccc23)cc1]

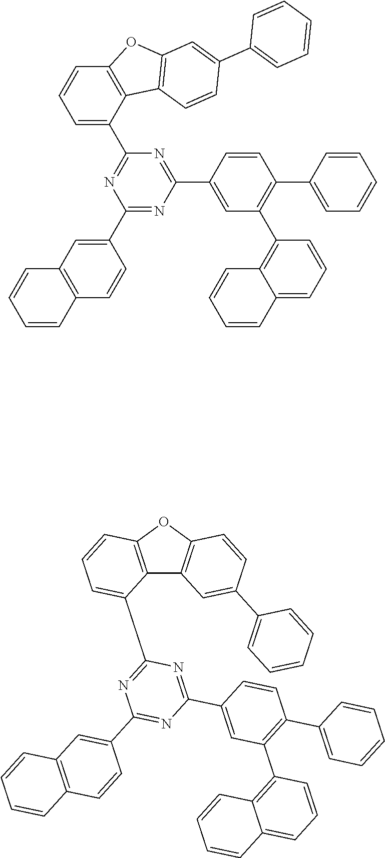 [SMILES: c1ccc(-c2ccc3c(c2)oc2cccc(-c4nc(-c5ccc(-c6ccccc6)c(-c6cccc7ccccc67)c5)nc(-c5ccc6ccccc6c5)n4)c23)cc1 ; c1ccc(-c2ccc3oc4cccc(-c5nc(-c6ccc(-c7ccccc7)c(-c7cccc8ccccc78)c6)nc(-c6ccc7ccccc7c6)n5)c4c3c2)cc1]

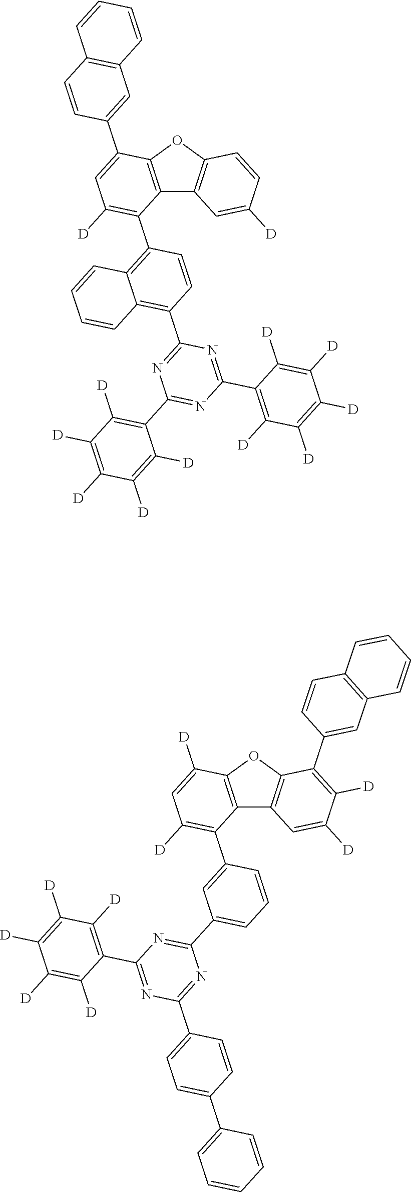 [SMILES: [2H]c1ccc2oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c3c2c1 ; [2H]c1cc2c(oc3c([2H])cc([2H])c(-c4cccc(-c5nc(-c6ccc(-c7ccccc7)cc6)nc(-c6c([2H])c([2H])c([2H])c([2H])c6[2H])n5)c4)c32)c(-c2ccc3ccccc3c2)c1[2H]]

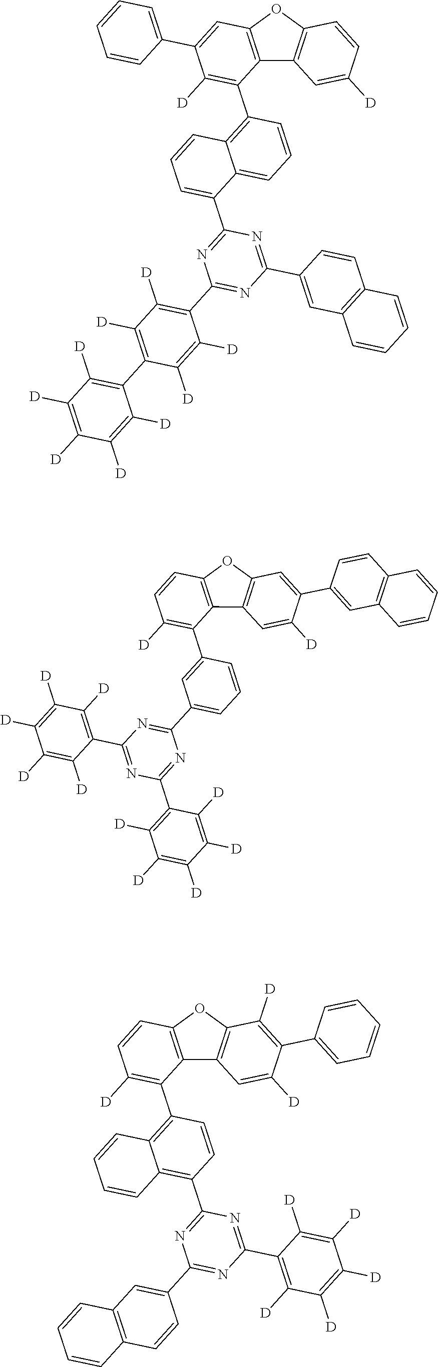 [SMILES: [2H]c1ccc2oc3cc(-c4ccccc4)c([2H])c(-c4cccc5c(-c6nc(-c7ccc8ccccc8c7)nc(-c7c([2H])c([2H])c(-c8c([2H])c([2H])c([2H])c([2H])c8[2H])c([2H])c7[2H])n6)cccc45)c3c2c1 ; [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; [2H]c1cc2c(oc3ccc([2H])c(-c4ccc(-c5nc(-c6ccc7ccccc7c6)nc(-c6c([2H])c([2H])c([2H])c([2H])c6[2H])n5)c5ccccc45)c32)c([2H])c1-c1ccccc1]

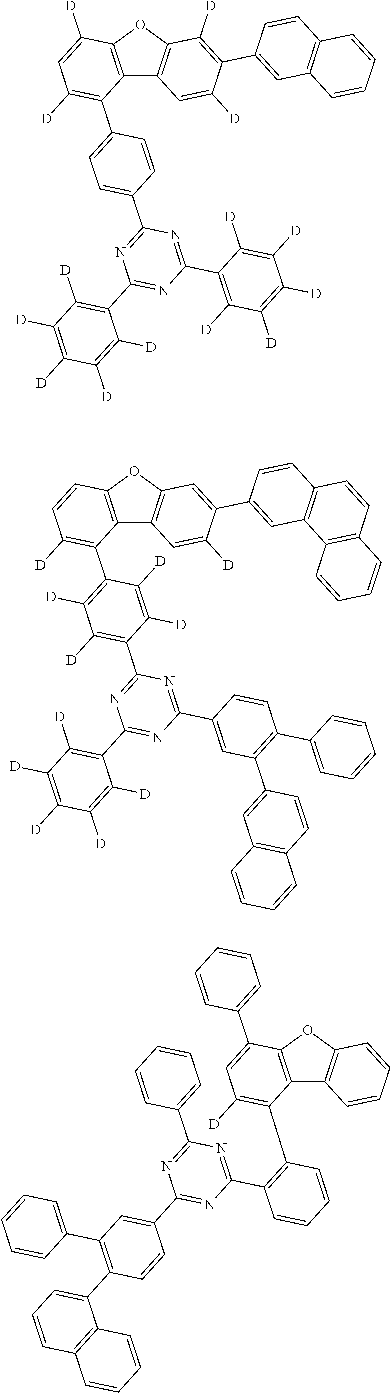 [SMILES: [2H]c1cc2c(oc3c([2H])cc([2H])c(-c4ccc(-c5nc(-c6c([2H])c([2H])c([2H])c([2H])c6[2H])nc(-c6c([2H])c([2H])c([2H])c([2H])c6[2H])n5)cc4)c32)c([2H])c1-c1ccc2ccccc2c1 ; [2H]c1cc2c(cc1-c1ccc3ccc4ccccc4c3c1)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12 ; [2H]c1cc(-c2ccccc2)c2oc3ccccc3c2c1-c1ccccc1-c1nc(-c2ccccc2)nc(-c2ccc(-c3cccc4ccccc34)c(-c3ccccc3)c2)n1]

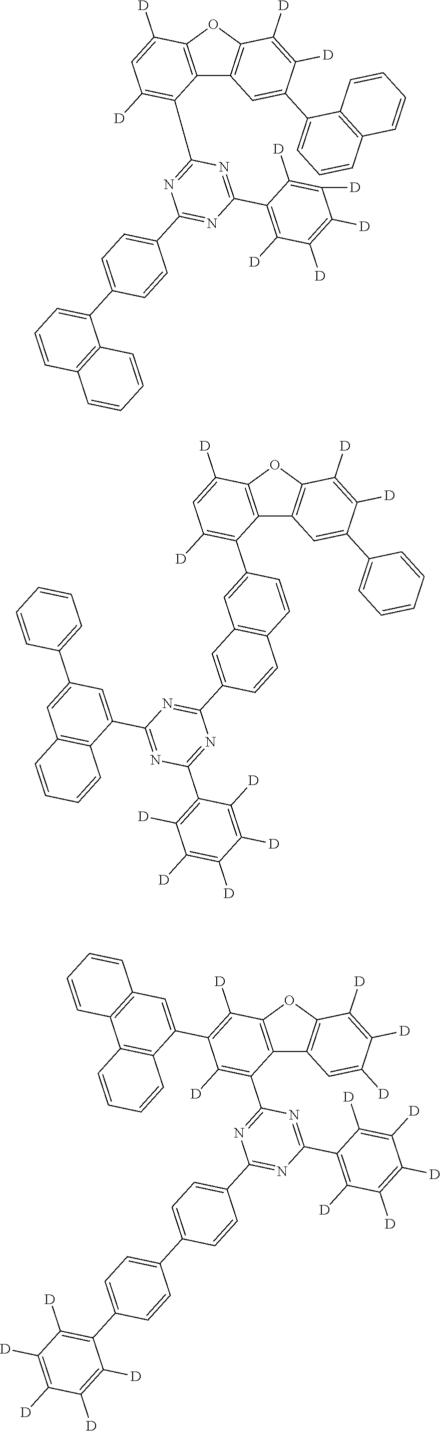 [SMILES: [2H]c1cc([2H])c2oc3c([2H])c([2H])c(-c4cccc5ccccc45)cc3c2c1-c1nc(-c2ccc(-c3cccc4ccccc34)cc2)nc(-c2c([2H])c([2H])c([2H])c([2H])c2[2H])n1 ; [2H]c1cc([2H])c2oc3c([2H])c([2H])c(-c4ccccc4)cc3c2c1-c1ccc2ccc(-c3nc(-c4c([2H])c([2H])c([2H])c([2H])c4[2H])nc(-c4cc(-c5ccccc5)cc5ccccc45)n3)cc2c1 ; [2H]c1cc2c(oc3c([2H])c(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c32)c([2H])c1[2H]]

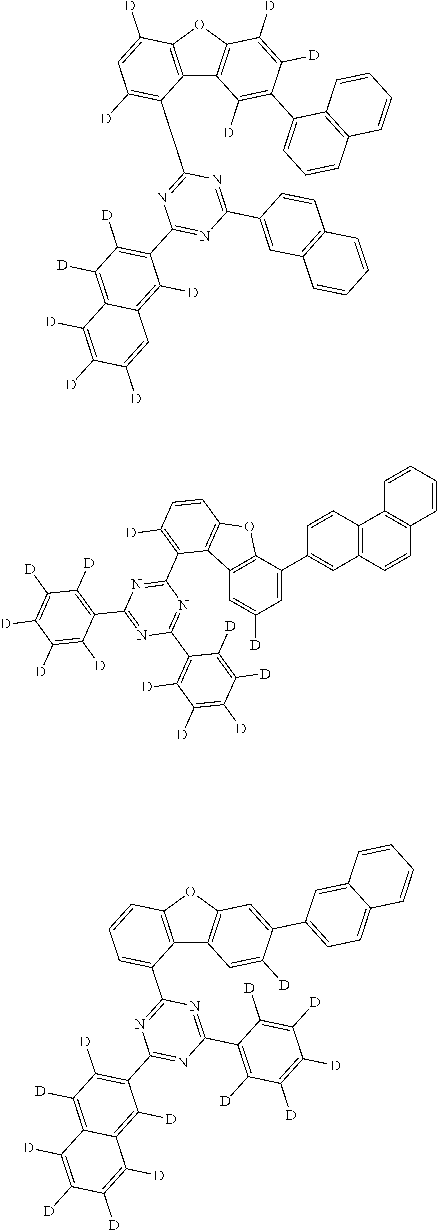 [SMILES: [2H]c1cc2c([2H])c(-c3nc(-c4ccc5ccccc5c4)nc(-c4c([2H])cc([2H])c5oc6c([2H])c([2H])c(-c7cccc8ccccc78)c([2H])c6c45)n3)c([2H])c([2H])c2c([2H])c1[2H] ; [2H]c1cc(-c2ccc3c(ccc4ccccc43)c2)c2oc3ccc([2H])c(-c4nc(-c5c([2H])c([2H])c([2H])c([2H])c5[2H])nc(-c5c([2H])c([2H])c([2H])c([2H])c5[2H])n4)c3c2c1 ; [2H]c1cc2c(cc1-c1ccc3ccccc3c1)oc1cccc(-c3nc(-c4c([2H])c([2H])c([2H])c([2H])c4[2H])nc(-c4c([2H])c([2H])c5c([2H])c([2H])c([2H])c([2H])c5c4[2H])n3)c12]

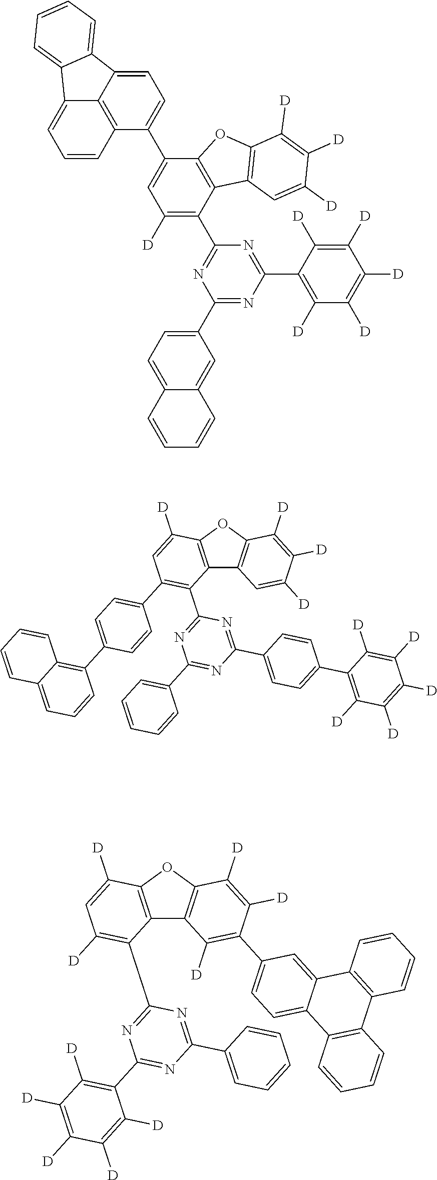 [SMILES: [2H]c1cc2c(oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c32)c([2H])c1[2H] ; [2H]c1cc2c(oc3c([2H])cc(-c4ccc(-c5cccc6ccccc56)cc4)c(-c4nc(-c5ccccc5)nc(-c5ccc(-c6c([2H])c([2H])c([2H])c([2H])c6[2H])cc5)n4)c32)c([2H])c1[2H] ; [2H]c1cc([2H])c2oc3c([2H])c([2H])c(-c4ccc5c6ccccc6c6ccccc6c5c4)c([2H])c3c2c1-c1nc(-c2ccccc2)nc(-c2c([2H])c([2H])c([2H])c([2H])c2[2H])n1]

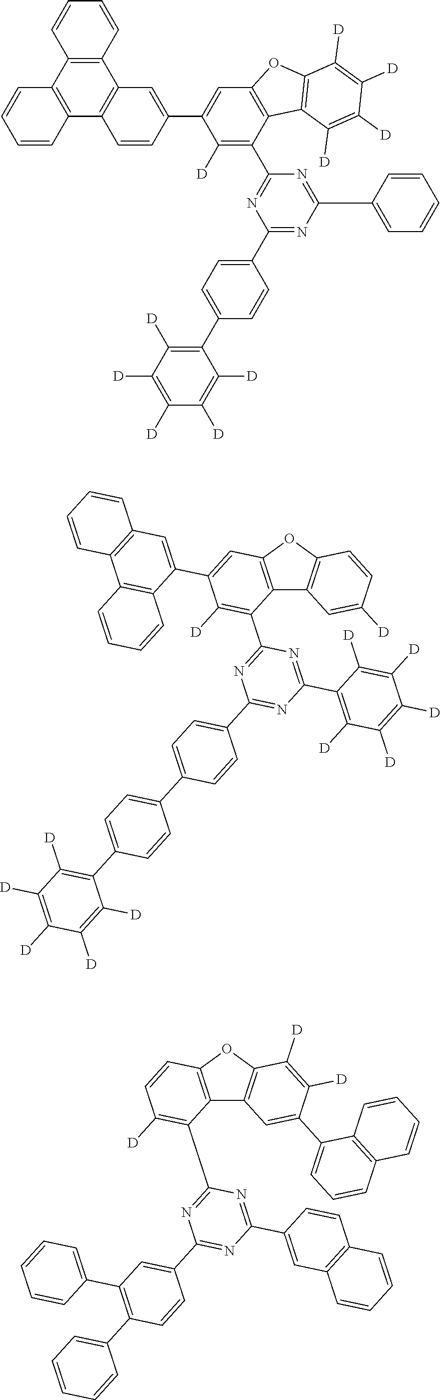 [SMILES: [2H]c1c([2H])c([2H])c(-c2ccc(-c3nc(-c4ccccc4)nc(-c4c([2H])c(-c5ccc6c7ccccc7c7ccccc7c6c5)cc5oc6c([2H])c([2H])c([2H])c([2H])c6c45)n3)cc2)c([2H])c1[2H] ; [2H]c1ccc2oc3cc(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c3c2c1 ; [2H]c1ccc2oc3c([2H])c([2H])c(-c4cccc5ccccc45)cc3c2c1-c1nc(-c2ccc(-c3ccccc3)c(-c3ccccc3)c2)nc(-c2ccc3ccccc3c2)n1]

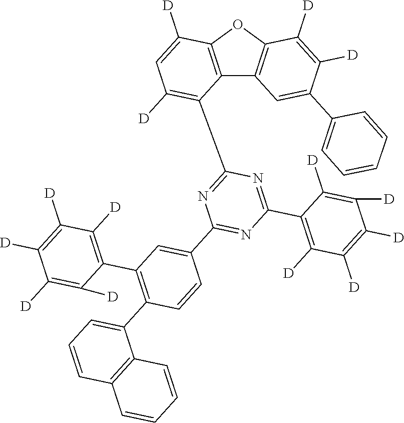 [SMILES: [2H]c1cc([2H])c2oc3c([2H])c([2H])c(-c4ccccc4)cc3c2c1-c1nc(-c2ccc(-c3cccc4ccccc34)c(-c3c([2H])c([2H])c([2H])c([2H])c3[2H])c2)nc(-c2c([2H])c([2H])c([2H])c([2H])c2[2H])n1]

The compound represented by Chemical Formula 1 can be prepared by a preparation method as shown in the following Reaction Scheme 1 as an example, and other remaining compounds can be prepared in a similar manner.

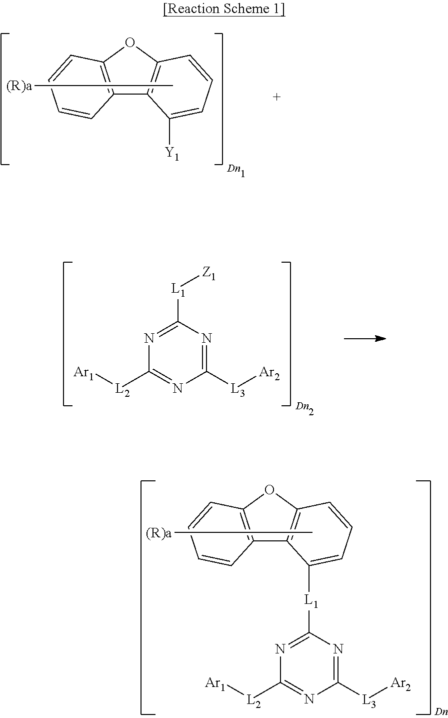

In Reaction Scheme 1, Ar₁, Ar₂, L₁ to L₃, R, Dn, n and a are the same as defined in Chemical Formula 1. Further, Y₁ is a boron-containing organic group, preferably a boronic acid group, a boronic acid ester group, or a boronic acid pinacol ester group, Z₁ is halogen, preferably Z₁ is chloro or bromo. Further, Dn₁ and Dn₂ represent a number of deuterium substitutions, and are each independently an integer of 0 or more and satisfy the condition of Dn₁+Dn₂=Dn.

Reaction Scheme 1 is a Suzuki coupling reaction, which is preferably carried out in the presence of a palladium catalyst and a base, and a reactive group for the Suzuki coupling reaction can be modified as known in the art.

The above preparation method may be further embodied in Preparation Examples described hereinafter.

Meanwhile, the compound represented by Chemical Formula 2 may be represented by one of the following Chemical Formulas 2-1 to 2-10:

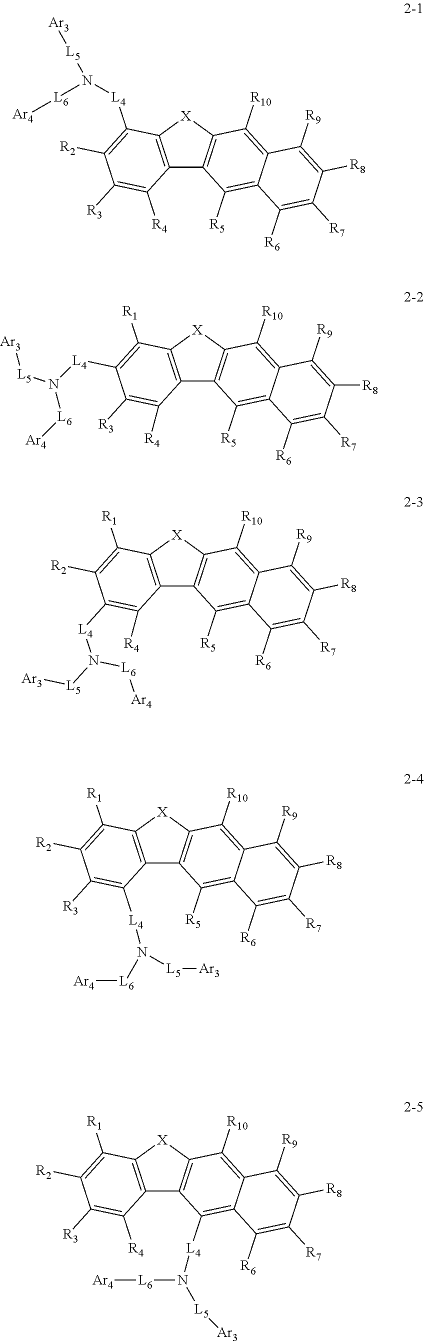

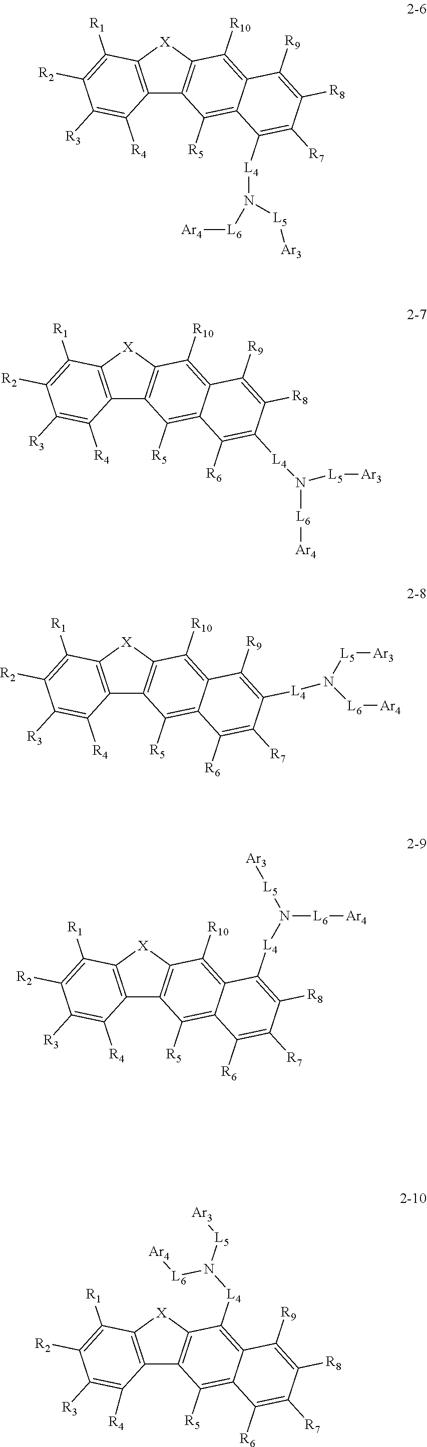

in Chemical Formulas 2-1 to 2-10,
X, R₁ to R₁₀, Ar₃, Ar₄, and L₄ to L₆ are the same as defined in Chemical Formula 2.

Preferably, Ar₃ and Ar₄ are each independently a substituted or unsubstituted C₆₋₂₀ aryl; or a substituted or unsubstituted C₂₋₂₀ heteroaryl containing one or more selected from the group consisting of N, O and S.

More preferably, Ar₃ and Ar₄ may be each independently phenyl, biphenyl, terphenyl, naphthyl, phenanthrenyl, triphenylenyl, (naphthyl)phenyl, (phenyl)naphthyl, fluorenyl, fluoranthenyl, dibenzofuranyl, dibenzothiophenyl, or carbazolyl, which is substituted or unsubstituted. Further, in this case, hydrogens of the Ar₃ and Ar₄ may be each independently substituted with deuterium; a C₁₋₂₀ alkyl such as methyl; a C₆₋₂₀ aryl such as phenyl or naphthyl; silyl; or arylsilyl such as triphenylsilyl.

Even more preferably, Ar₃ and Ar₄ are each independently phenyl, biphenyl, terphenyl, naphthyl, phenanthrenyl, triphenylenyl, (naphthyl)phenyl, (phenyl)naphthyl, fluorenyl, dimethyl fluorenyl, diphenylfluorenyl, fluoranthenyl, dibenzofuranyl, dibenzothiophenyl, carbazolyl, or 9-phenylcarbazolyl, each of which may be unsubstituted or substituted with deuterium, phenyl, or triphenylsilyl.

Still more preferably, Ar₃ and Ar₄ may each independently be one selected from the following:

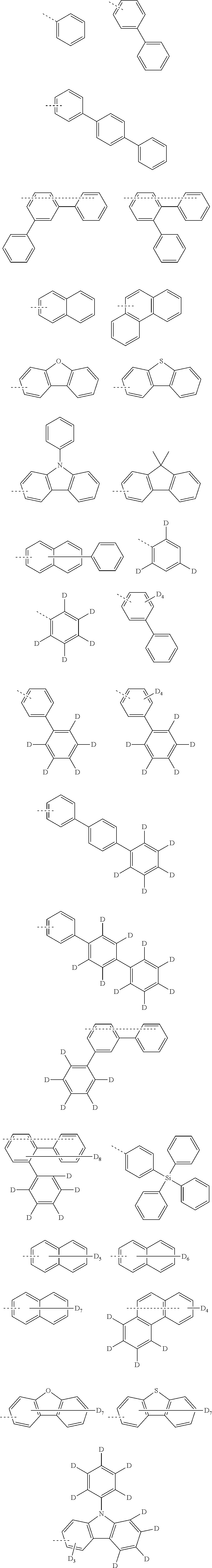

in each of the above Chemical Formulas, the dotted line represents a bonding position.

Further, Ar₃ and Ar₄ may be the same as each other or may be different from each other.

Preferably, L₄ may be a single bond; a substituted or unsubstituted C₆₋₂₀ arylene; or a substituted or unsubstituted C₂₋₂₀ heteroarylene containing one or more selected from the group consisting of N, O and S, and when the L₄ is substituted, it may be substituted with one or more deuterium; a C₁₋₂₀ alkyl such as methyl; or a C₆₋₂₀ aryl such as phenyl or naphthyl.

More preferably, L₄ is a single bond, phenylene, biphenyldiyl, naphthalenediyl or binaphthalenediyl, and hydrogens of the L₄ may be each independently unsubstituted or substituted with deuterium, phenyl, or naphthyl.

Even more preferably, L₄ may be one selected from the following:

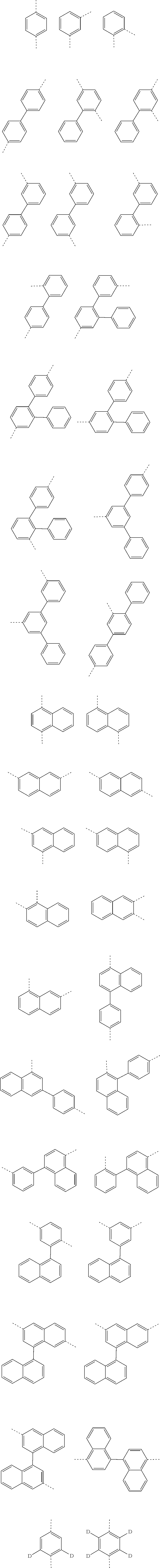

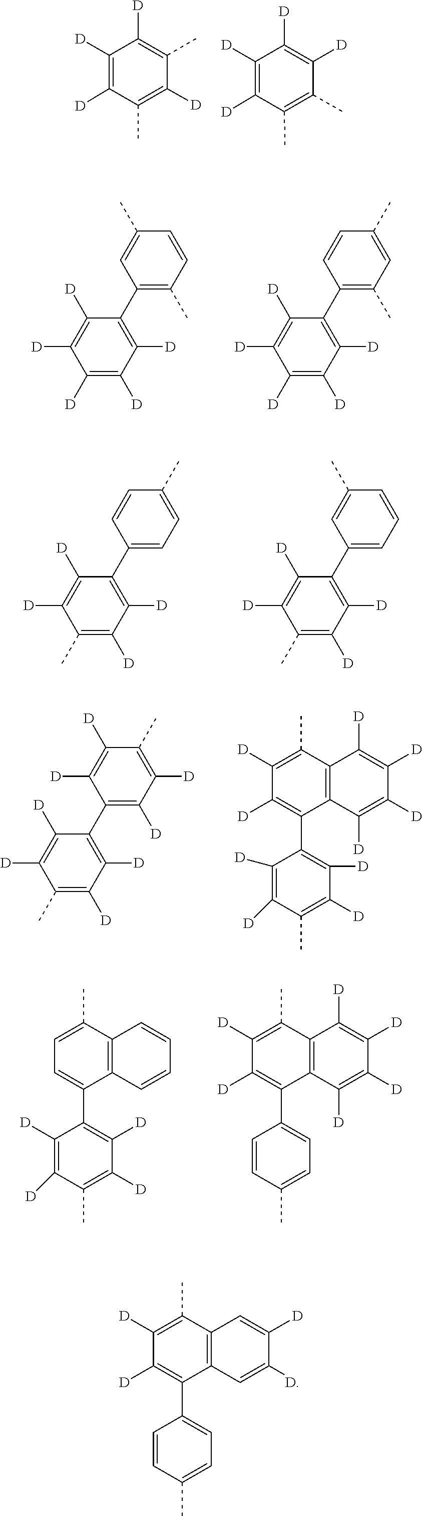

in each of Chemical Formulas, the dotted line represents a bonding position.

Further, preferably, L₅ and L₆ may be each independently a single bond; a substituted or unsubstituted C₆₋₂₀ arylene; or a substituted or unsubstituted C₂₋₂₀ heteroarylene containing one or more selected from the group consisting of N, O and S. When the L₅ or L₆ is substituted, it may be substituted with one or more deuterium; a C₁₋₂₀ alkyl such as methyl; or a C₆₋₂₀ aryl such as phenyl or naphthyl.

More preferably, L₅ and L₆ may be each independently a single bond, phenylene, biphenyldiyl, naphthalenediyl or binaphthalenediyl, each of which, except for a single bond, may be unsubstituted or substituted with deuterium, phenyl, or naphthyl.

Even more preferably, L₅ and L₆ may each independently be one selected from the following:

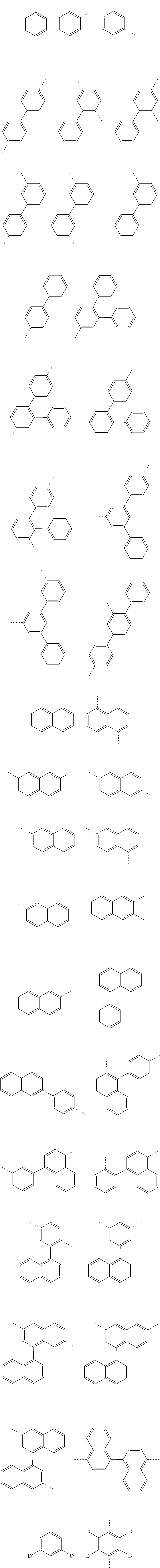

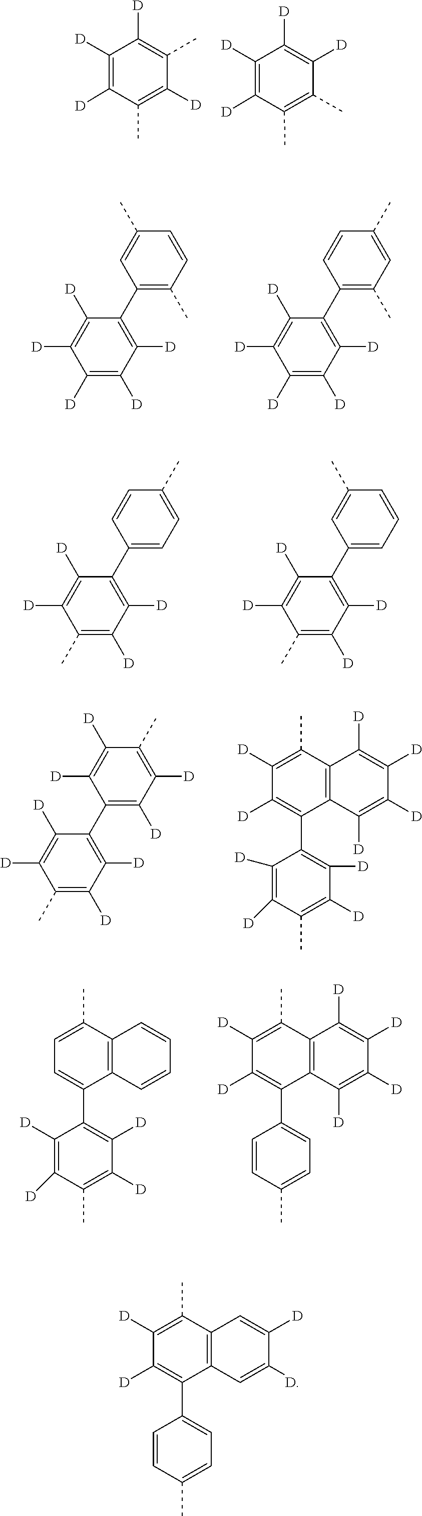

in each of Chemical Formulas, the dotted line represents a bonding position.

Further, the L₅ and L₆ may be the same as each other or may be different from each other.

Representative examples of the compound represented by Chemical Formula 2 are as follows:

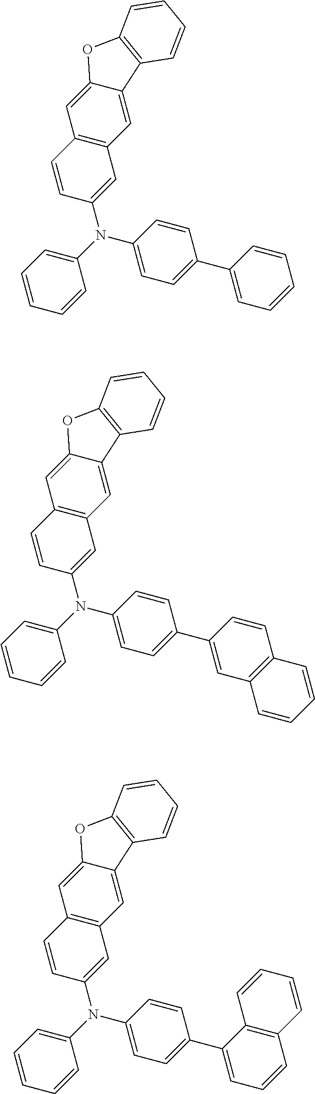 [SMILES: c1ccc(-c2ccc(N(c3ccccc3)c3ccc4cc5oc6ccccc6c5cc4c3)cc2)cc1 ; c1ccc(N(c2ccc(-c3ccc4ccccc4c3)cc2)c2ccc3cc4oc5ccccc5c4cc3c2)cc1 ; c1ccc(N(c2ccc(-c3cccc4ccccc34)cc2)c2ccc3cc4oc5ccccc5c4cc3c2)cc1]

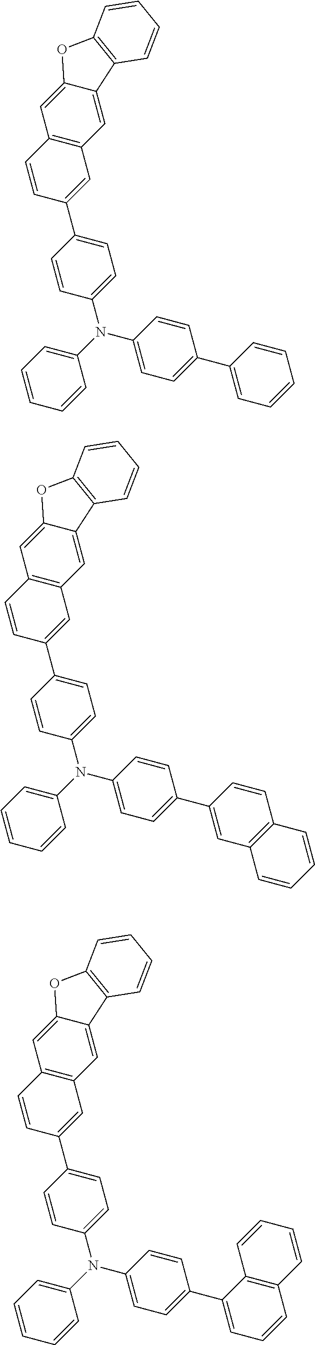

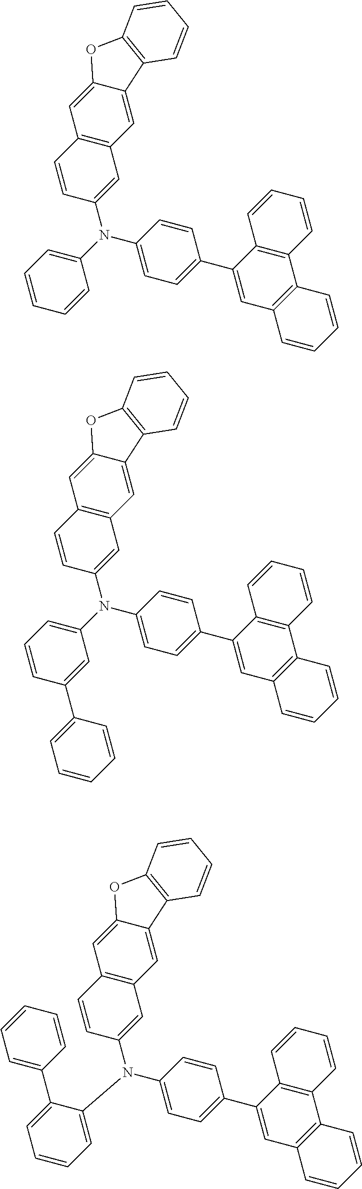 [SMILES: c1ccc(N(c2ccc(-c3cc4ccccc4c4ccccc34)cc2)c2ccc3cc4oc5ccccc5c4cc3c2)cc1 ; c1ccc(-c2cccc(N(c3ccc(-c4cc5ccccc5c5ccccc45)cc3)c3ccc4cc5oc6ccccc6c5cc4c3)c2)cc1 ; c1ccc(-c2ccccc2N(c2ccc(-c3cc4ccccc4c4ccccc34)cc2)c2ccc3cc4oc5ccccc5c4cc3c2)cc1]

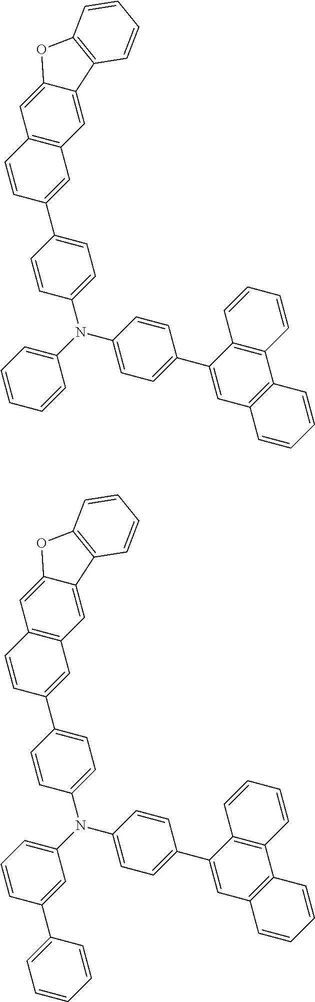 [SMILES: c1ccc(N(c2ccc(-c3ccc4cc5oc6ccccc6c5cc4c3)cc2)c2ccc(-c3cc4ccccc4c4ccccc34)cc2)cc1 ; c1ccc(-c2cccc(N(c3ccc(-c4ccc5cc6oc7ccccc7c6cc5c4)cc3)c3ccc(-c4cc5ccccc5c5ccccc45)cc3)c2)cc1]

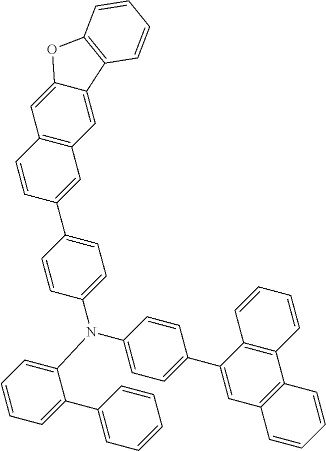 [SMILES: c1ccc(-c2ccccc2N(c2ccc(-c3ccc4cc5oc6ccccc6c5cc4c3)cc2)c2ccc(-c3cc4ccccc4c4ccccc34)cc2)cc1]

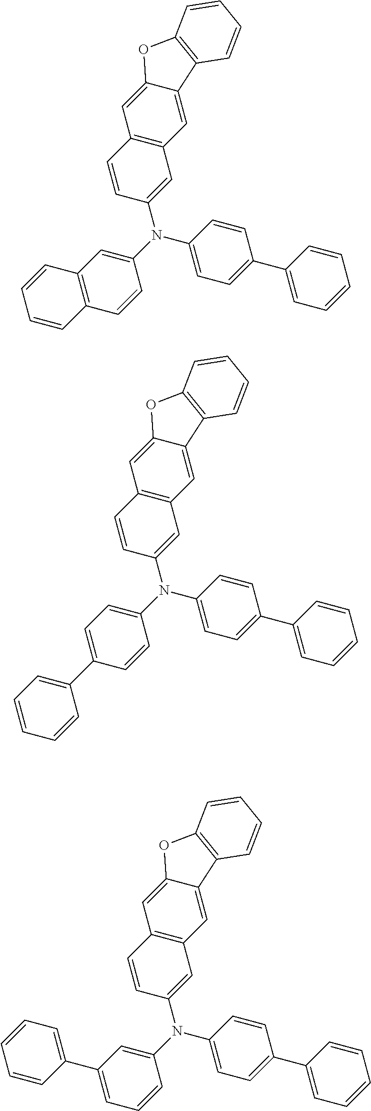 [SMILES: c1ccc(-c2ccc(N(c3ccc4ccccc4c3)c3ccc4cc5oc6ccccc6c5cc4c3)cc2)cc1 ; c1ccc(-c2ccc(N(c3ccc(-c4ccccc4)cc3)c3ccc4cc5oc6ccccc6c5cc4c3)cc2)cc1 ; c1ccc(-c2ccc(N(c3cccc(-c4ccccc4)c3)c3ccc4cc5oc6ccccc6c5cc4c3)cc2)cc1]

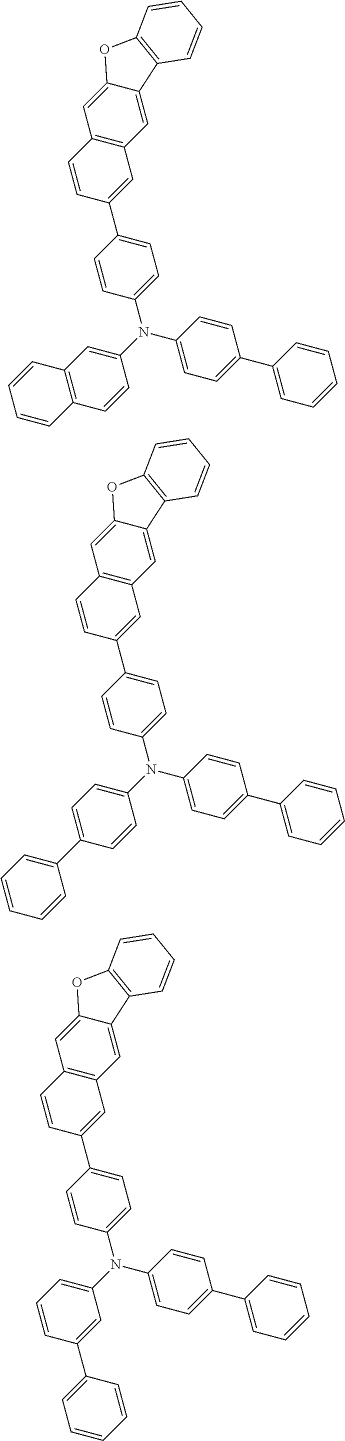 [SMILES: c1ccc(-c2ccc(N(c3ccc(-c4ccc5cc6oc7ccccc7c6cc5c4)cc3)c3ccc4ccccc4c3)cc2)cc1 ; c1ccc(-c2ccc(N(c3ccc(-c4ccccc4)cc3)c3ccc(-c4ccc5cc6oc7ccccc7c6cc5c4)cc3)cc2)cc1 ; c1ccc(-c2ccc(N(c3ccc(-c4ccc5cc6oc7ccccc7c6cc5c4)cc3)c3cccc(-c4ccccc4)c3)cc2)cc1]

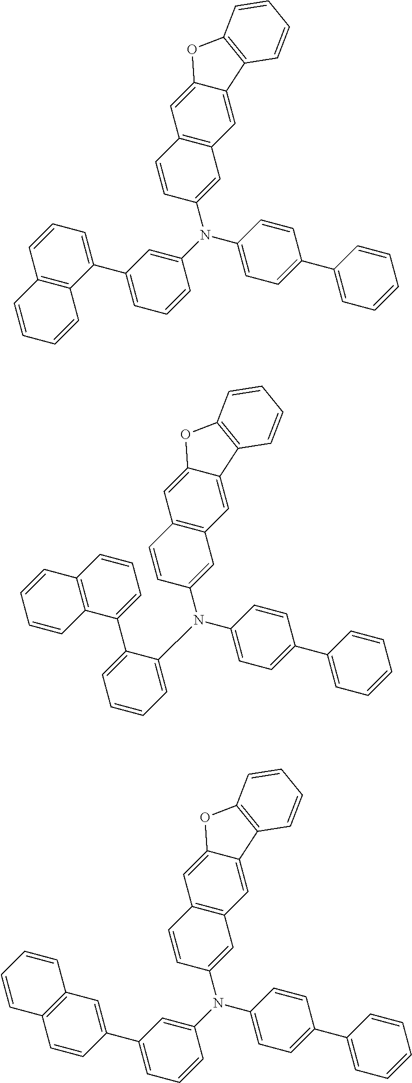 [SMILES: c1ccc(-c2ccc(N(c3cccc(-c4cccc5ccccc45)c3)c3ccc4cc5oc6ccccc6c5cc4c3)cc2)cc1 ; c1ccc(-c2ccc(N(c3ccc4cc5oc6ccccc6c5cc4c3)c3ccccc3-c3cccc4ccccc34)cc2)cc1 ; c1ccc(-c2ccc(N(c3cccc(-c4ccc5ccccc5c4)c3)c3ccc4cc5oc6ccccc6c5cc4c3)cc2)cc1]

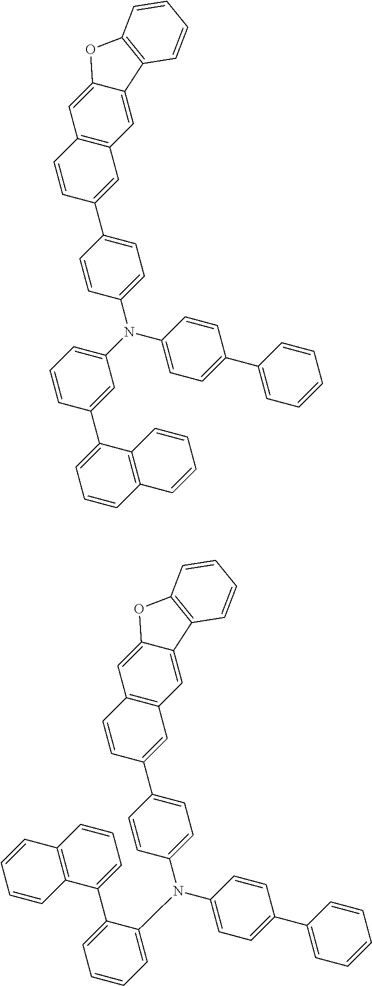 [SMILES: c1ccc(-c2ccc(N(c3ccc(-c4ccc5cc6oc7ccccc7c6cc5c4)cc3)c3cccc(-c4cccc5ccccc45)c3)cc2)cc1 ; c1ccc(-c2ccc(N(c3ccc(-c4ccc5cc6oc7ccccc7c6cc5c4)cc3)c3ccccc3-c3cccc4ccccc34)cc2)cc1]

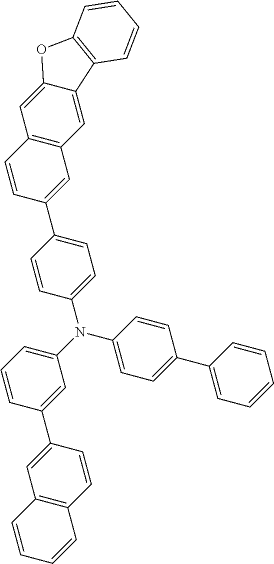 [SMILES: c1ccc(-c2ccc(N(c3ccc(-c4ccc5cc6oc7ccccc7c6cc5c4)cc3)c3cccc(-c4ccc5ccccc5c4)c3)cc2)cc1]

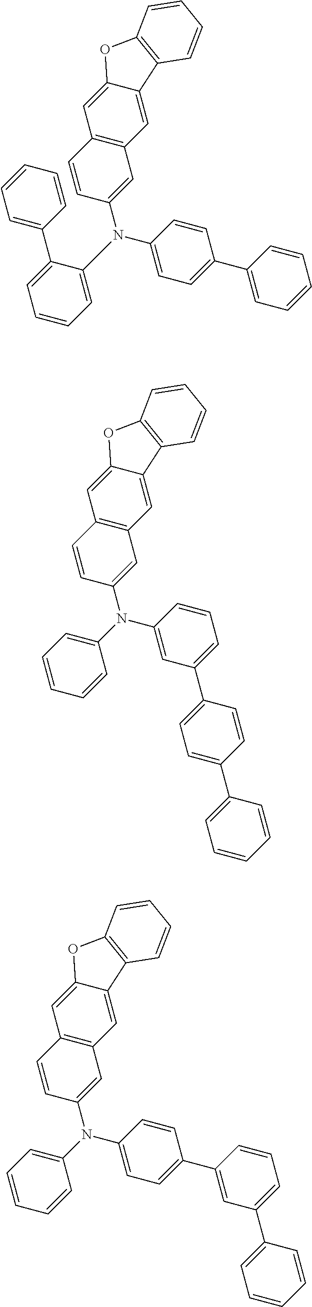 [SMILES: c1ccc(-c2ccc(N(c3ccc4cc5oc6ccccc6c5cc4c3)c3ccccc3-c3ccccc3)cc2)cc1 ; c1ccc(-c2ccc(-c3cccc(N(c4ccccc4)c4ccc5cc6oc7ccccc7c6cc5c4)c3)cc2)cc1 ; c1ccc(-c2cccc(-c3ccc(N(c4ccccc4)c4ccc5cc6oc7ccccc7c6cc5c4)cc3)c2)cc1]

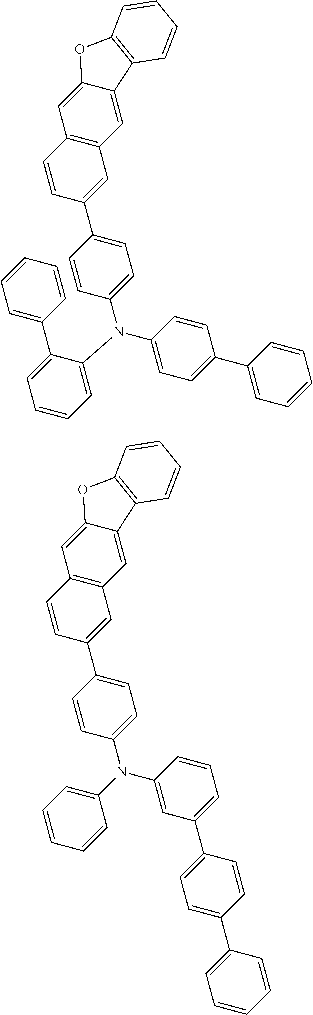 [SMILES: c1ccc(-c2ccc(N(c3ccc(-c4ccc5cc6oc7ccccc7c6cc5c4)cc3)c3ccccc3-c3ccccc3)cc2)cc1 ; c1ccc(-c2ccc(-c3cccc(N(c4ccccc4)c4ccc(-c5ccc6cc7oc8ccccc8c7cc6c5)cc4)c3)cc2)cc1]

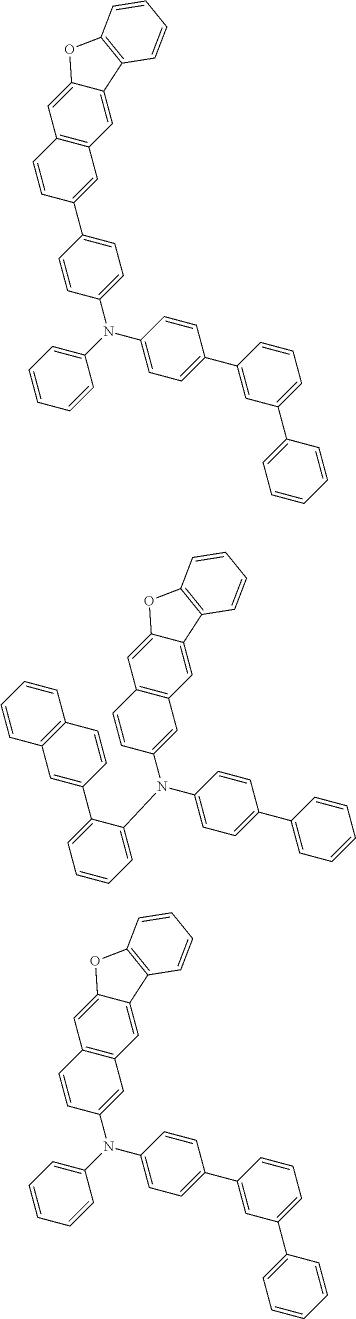

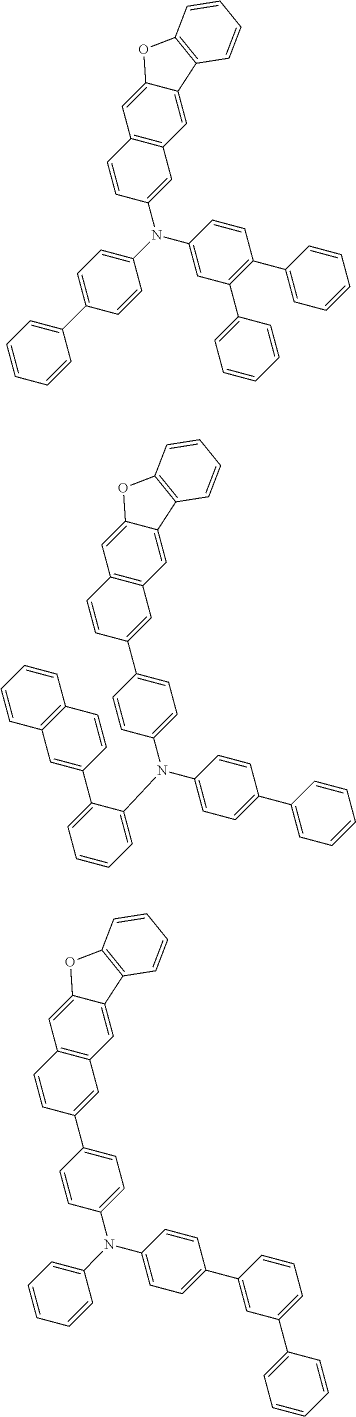 [SMILES: c1ccc(-c2ccc(N(c3ccc(-c4ccccc4)c(-c4ccccc4)c3)c3ccc4cc5oc6ccccc6c5cc4c3)cc2)cc1 ; c1ccc(-c2ccc(N(c3ccc(-c4ccc5cc6oc7ccccc7c6cc5c4)cc3)c3ccccc3-c3ccc4ccccc4c3)cc2)cc1 ; c1ccc(-c2cccc(-c3ccc(N(c4ccccc4)c4ccc(-c5ccc6cc7oc8ccccc8c7cc6c5)cc4)cc3)c2)cc1]

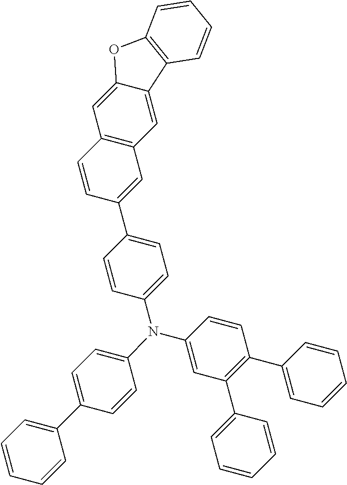 [SMILES: c1ccc(-c2ccc(N(c3ccc(-c4ccc5cc6oc7ccccc7c6cc5c4)cc3)c3ccc(-c4ccccc4)c(-c4ccccc4)c3)cc2)cc1]

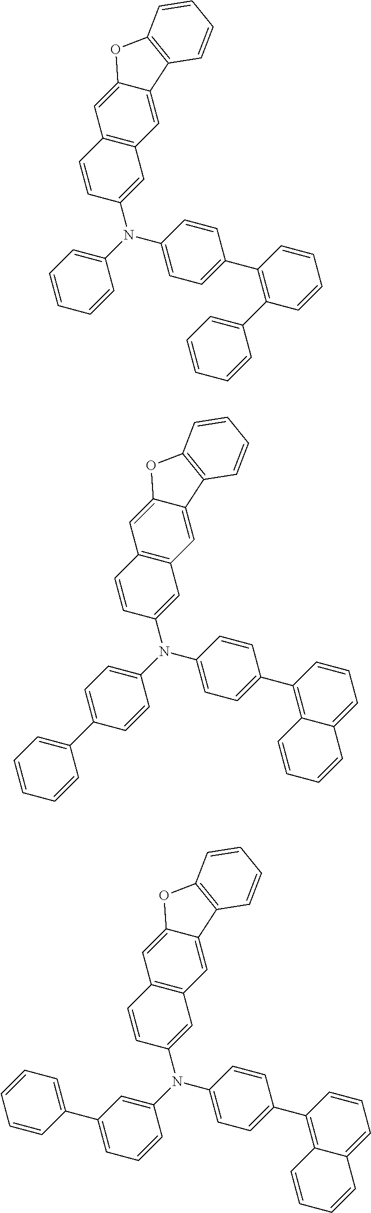

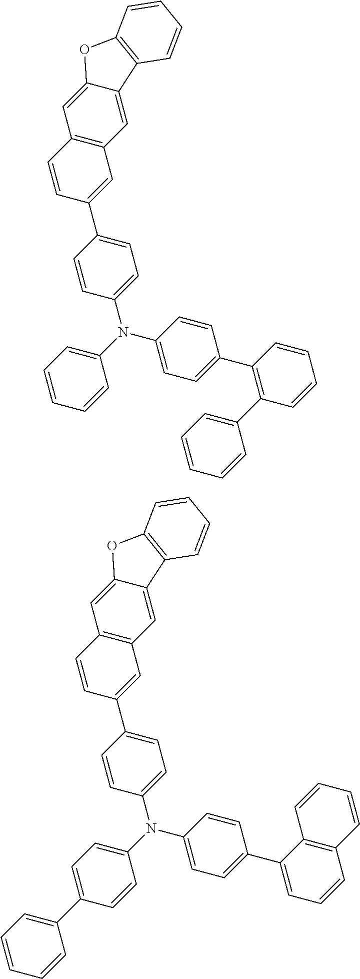

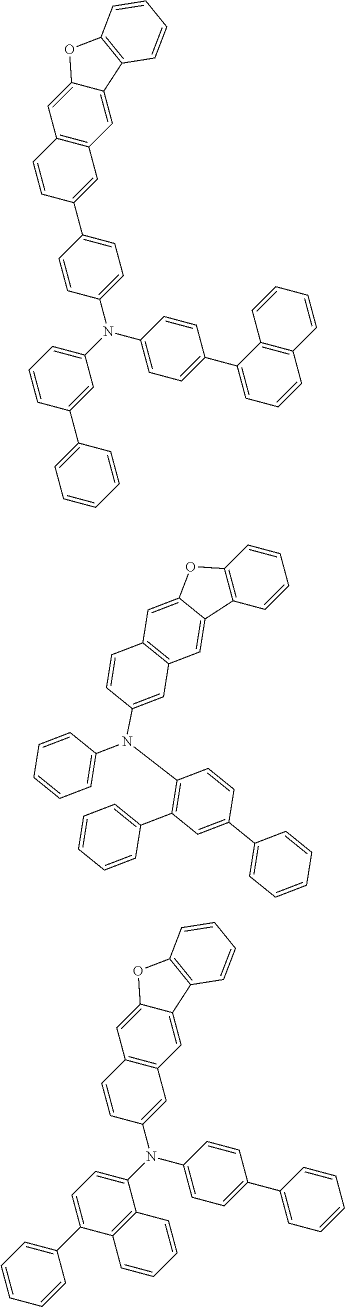 [SMILES: c1ccc(-c2cccc(N(c3ccc(-c4ccc5cc6oc7ccccc7c6cc5c4)cc3)c3ccc(-c4cccc5ccccc45)cc3)c2)cc1 ; c1ccc(-c2ccc(N(c3ccccc3)c3ccc4cc5oc6ccccc6c5cc4c3)c(-c3ccccc3)c2)cc1 ; c1ccc(-c2ccc(N(c3ccc4cc5oc6ccccc6c5cc4c3)c3ccc(-c4ccccc4)c4ccccc34)cc2)cc1]

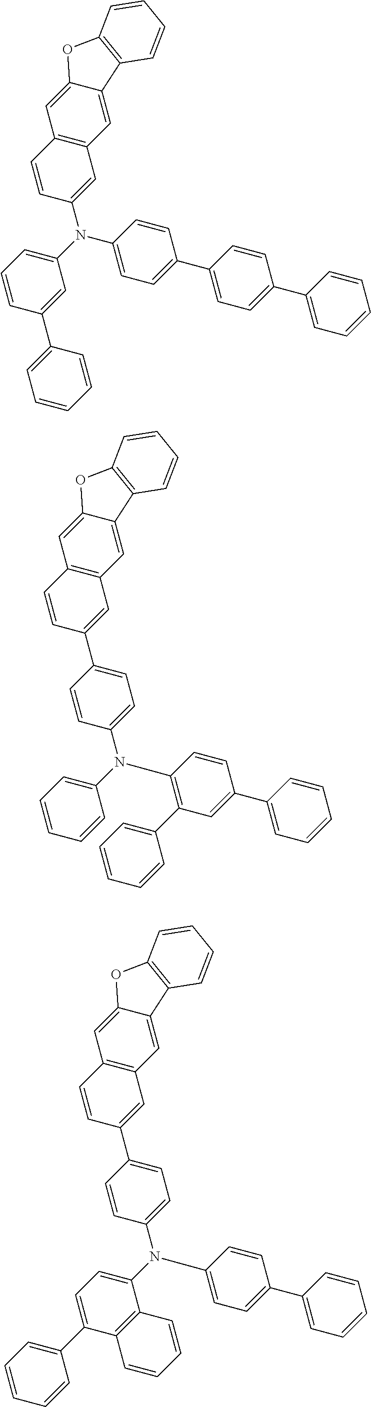 [SMILES: c1ccc(-c2ccc(-c3ccc(N(c4cccc(-c5ccccc5)c4)c4ccc5cc6oc7ccccc7c6cc5c4)cc3)cc2)cc1 ; c1ccc(-c2ccc(N(c3ccccc3)c3ccc(-c4ccc5cc6oc7ccccc7c6cc5c4)cc3)c(-c3ccccc3)c2)cc1 ; c1ccc(-c2ccc(N(c3ccc(-c4ccc5cc6oc7ccccc7c6cc5c4)cc3)c3ccc(-c4ccccc4)c4ccccc34)cc2)cc1]

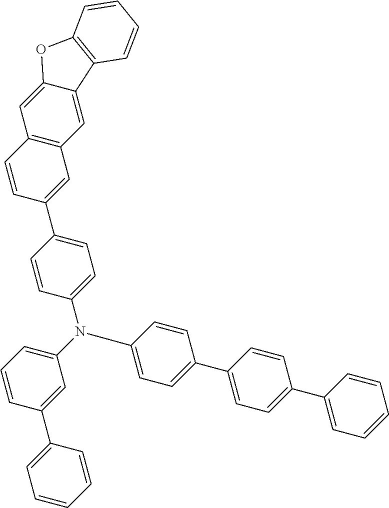 [SMILES: c1ccc(-c2ccc(-c3ccc(N(c4ccc(-c5ccc6cc7oc8ccccc8c7cc6c5)cc4)c4cccc(-c5ccccc5)c4)cc3)cc2)cc1]

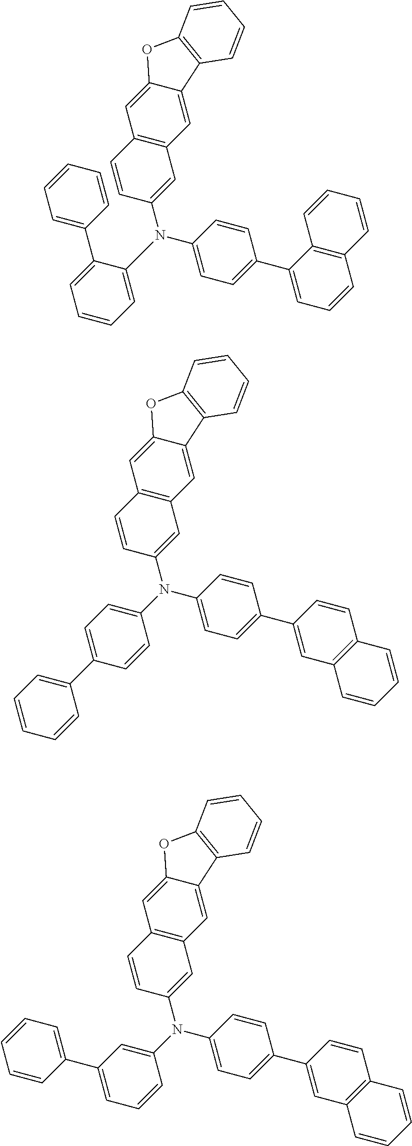 [SMILES: c1ccc(-c2ccccc2N(c2ccc(-c3cccc4ccccc34)cc2)c2ccc3cc4oc5ccccc5c4cc3c2)cc1 ; c1ccc(-c2ccc(N(c3ccc(-c4ccc5ccccc5c4)cc3)c3ccc4cc5oc6ccccc6c5cc4c3)cc2)cc1 ; c1ccc(-c2cccc(N(c3ccc(-c4ccc5ccccc5c4)cc3)c3ccc4cc5oc6ccccc6c5cc4c3)c2)cc1]

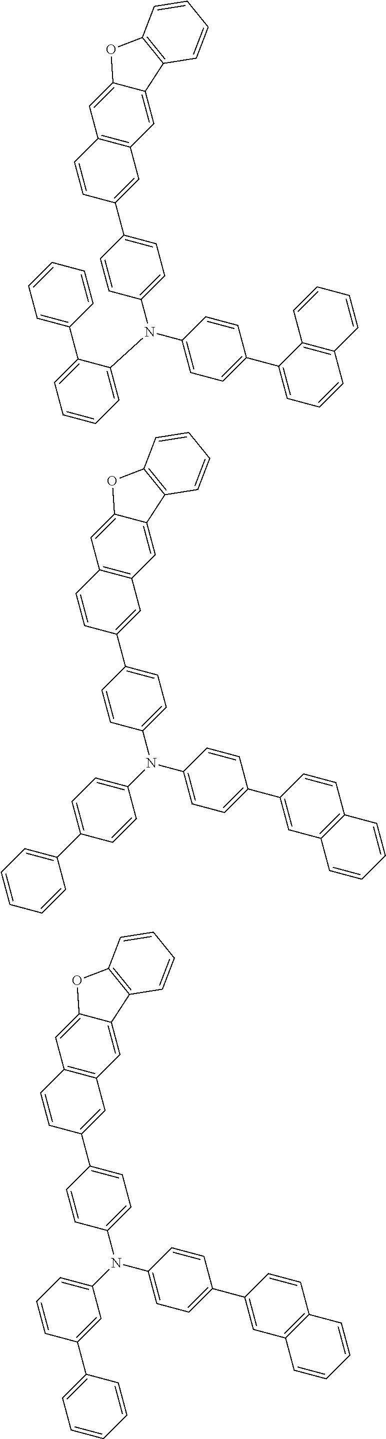 [SMILES: c1ccc(-c2ccccc2N(c2ccc(-c3ccc4cc5oc6ccccc6c5cc4c3)cc2)c2ccc(-c3cccc4ccccc34)cc2)cc1 ; c1ccc(-c2ccc(N(c3ccc(-c4ccc5ccccc5c4)cc3)c3ccc(-c4ccc5cc6oc7ccccc7c6cc5c4)cc3)cc2)cc1 ; c1ccc(-c2cccc(N(c3ccc(-c4ccc5ccccc5c4)cc3)c3ccc(-c4ccc5cc6oc7ccccc7c6cc5c4)cc3)c2)cc1]

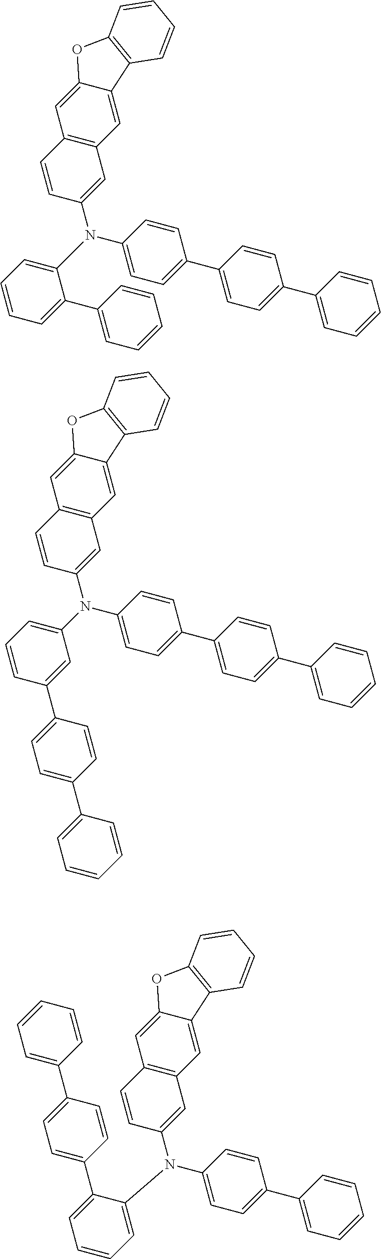

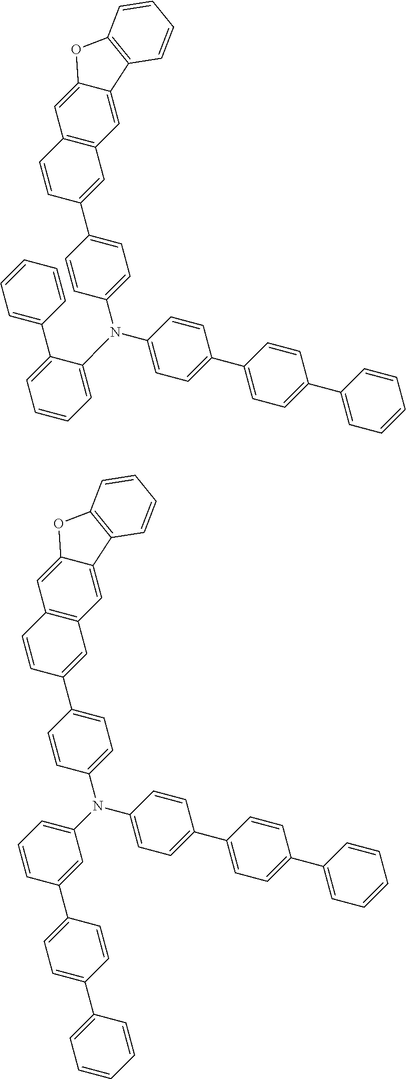 [SMILES: c1ccc(-c2ccc(-c3ccc(N(c4ccc(-c5ccc6cc7oc8ccccc8c7cc6c5)cc4)c4ccccc4-c4ccccc4)cc3)cc2)cc1 ; c1ccc(-c2ccc(-c3ccc(N(c4ccc(-c5ccc6cc7oc8ccccc8c7cc6c5)cc4)c4cccc(-c5ccc(-c6ccccc6)cc5)c4)cc3)cc2)cc1]

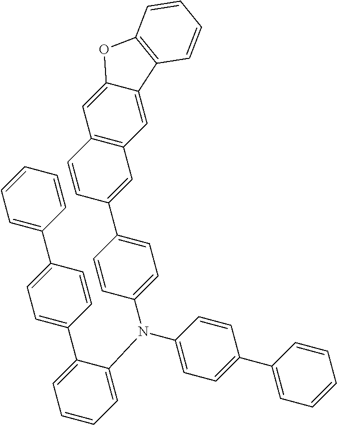 [SMILES: c1ccc(-c2ccc(-c3ccccc3N(c3ccc(-c4ccccc4)cc3)c3ccc(-c4ccc5cc6oc7ccccc7c6cc5c4)cc3)cc2)cc1]

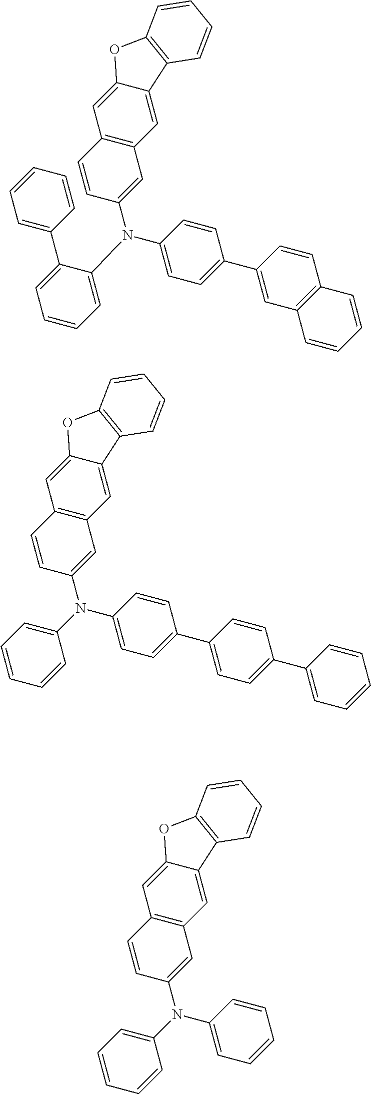 [SMILES: c1ccc(-c2ccccc2N(c2ccc(-c3ccc4ccccc4c3)cc2)c2ccc3cc4oc5ccccc5c4cc3c2)cc1 ; c1ccc(-c2ccc(-c3ccc(N(c4ccccc4)c4ccc5cc6oc7ccccc7c6cc5c4)cc3)cc2)cc1 ; c1ccc(N(c2ccccc2)c2ccc3cc4oc5ccccc5c4cc3c2)cc1]

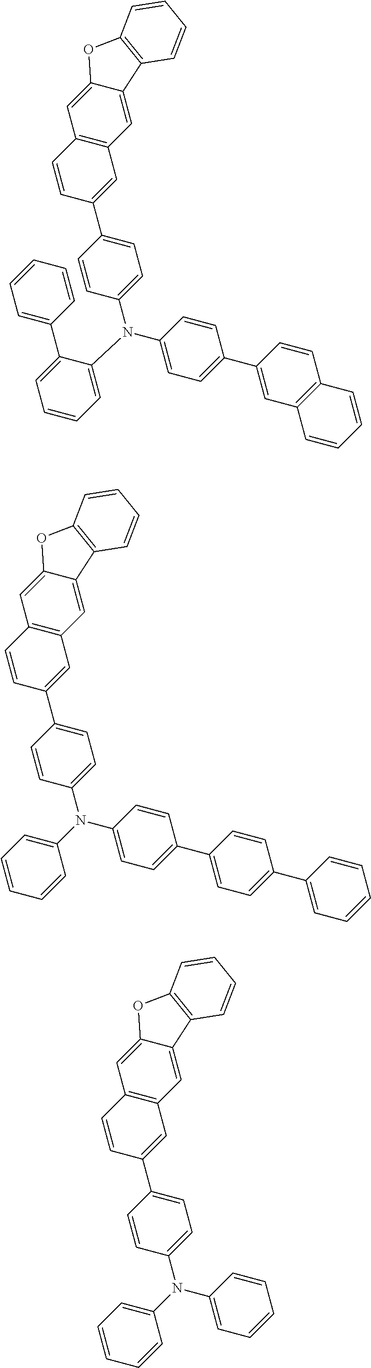 [SMILES: c1ccc(-c2ccccc2N(c2ccc(-c3ccc4ccccc4c3)cc2)c2ccc(-c3ccc4cc5oc6ccccc6c5cc4c3)cc2)cc1 ; c1ccc(-c2ccc(-c3ccc(N(c4ccccc4)c4ccc(-c5ccc6cc7oc8ccccc8c7cc6c5)cc4)cc3)cc2)cc1 ; c1ccc(N(c2ccccc2)c2ccc(-c3ccc4cc5oc6ccccc6c5cc4c3)cc2)cc1]

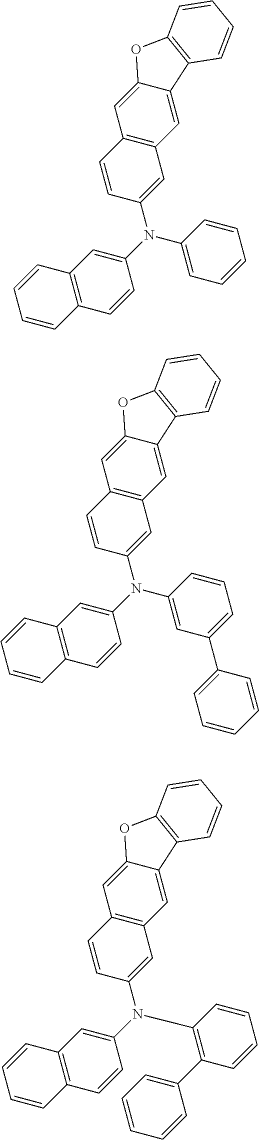 [SMILES: c1ccc(N(c2ccc3ccccc3c2)c2ccc3cc4oc5ccccc5c4cc3c2)cc1 ; c1ccc(-c2cccc(N(c3ccc4ccccc4c3)c3ccc4cc5oc6ccccc6c5cc4c3)c2)cc1 ; c1ccc(-c2ccccc2N(c2ccc3ccccc3c2)c2ccc3cc4oc5ccccc5c4cc3c2)cc1]

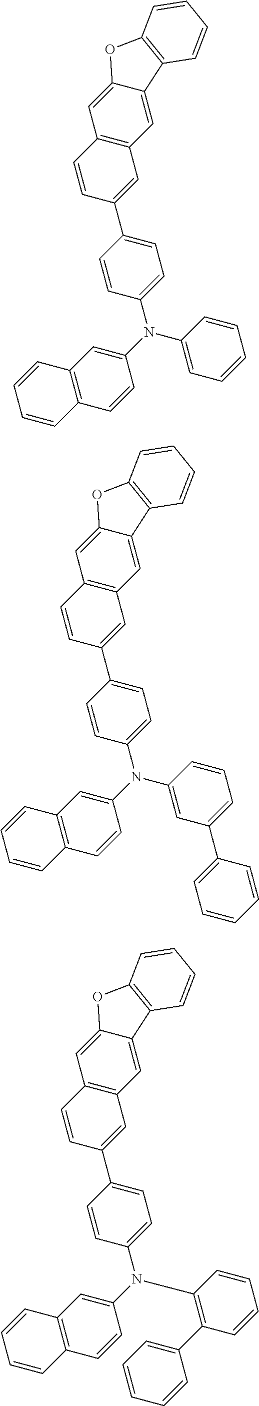

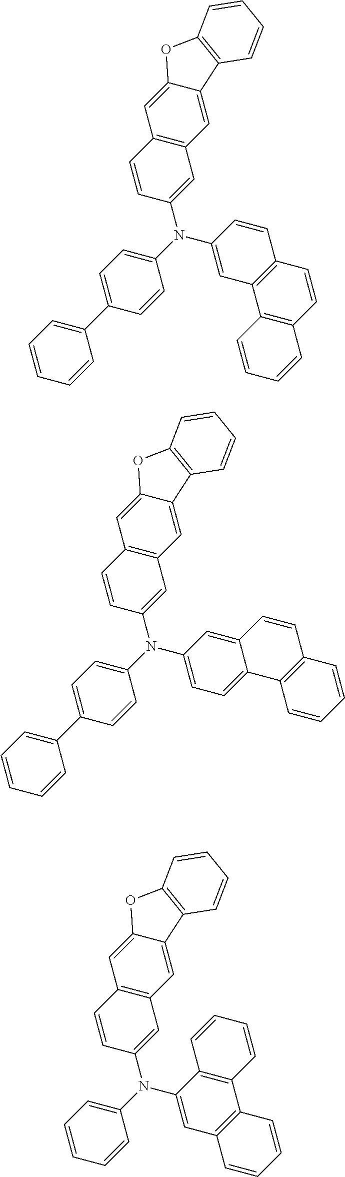 [SMILES: c1ccc(-c2ccc(N(c3ccc4cc5oc6ccccc6c5cc4c3)c3ccc4ccc5ccccc5c4c3)cc2)cc1 ; c1ccc(-c2ccc(N(c3ccc4cc5oc6ccccc6c5cc4c3)c3ccc4c(ccc5ccccc54)c3)cc2)cc1 ; c1ccc(N(c2ccc3cc4oc5ccccc5c4cc3c2)c2cc3ccccc3c3ccccc23)cc1]

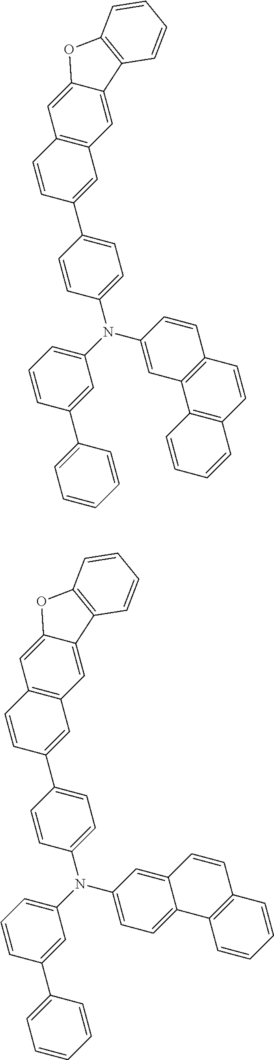 [SMILES: c1ccc(-c2cccc(N(c3ccc(-c4ccc5cc6oc7ccccc7c6cc5c4)cc3)c3ccc4ccc5ccccc5c4c3)c2)cc1 ; c1ccc(-c2cccc(N(c3ccc(-c4ccc5cc6oc7ccccc7c6cc5c4)cc3)c3ccc4c(ccc5ccccc54)c3)c2)cc1]

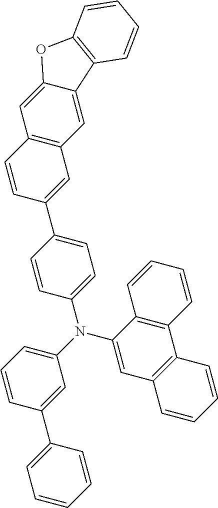 [SMILES: c1ccc(-c2cccc(N(c3ccc(-c4ccc5cc6oc7ccccc7c6cc5c4)cc3)c3cc4ccccc4c4ccccc34)c2)cc1]

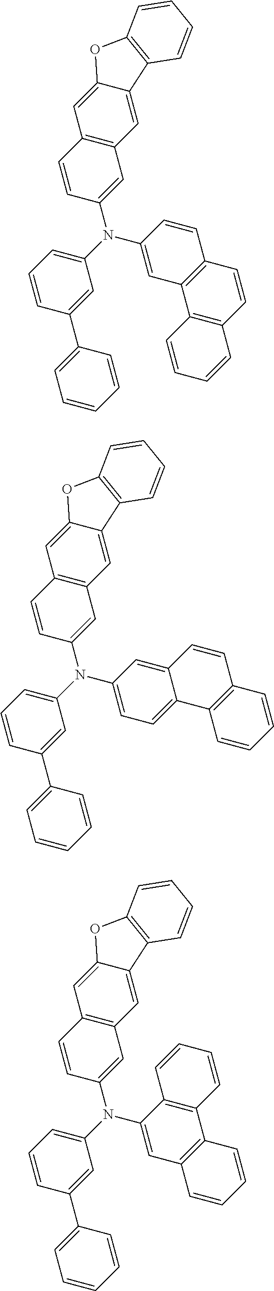

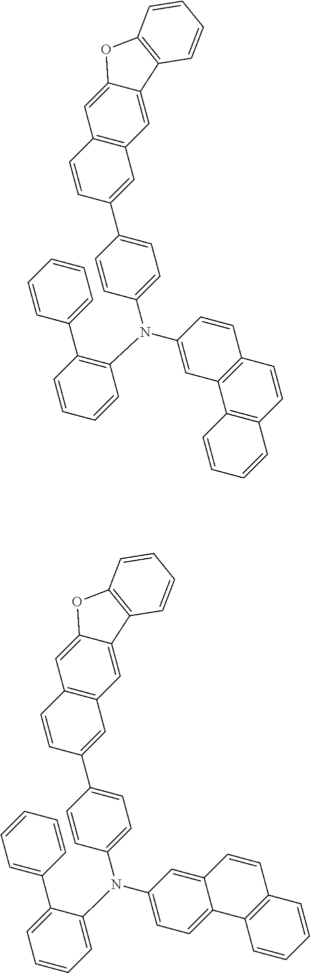 [SMILES: c1ccc(-c2ccccc2N(c2ccc(-c3ccc4cc5oc6ccccc6c5cc4c3)cc2)c2ccc3ccc4ccccc4c3c2)cc1 ; c1ccc(-c2ccccc2N(c2ccc(-c3ccc4cc5oc6ccccc6c5cc4c3)cc2)c2ccc3c(ccc4ccccc43)c2)cc1]

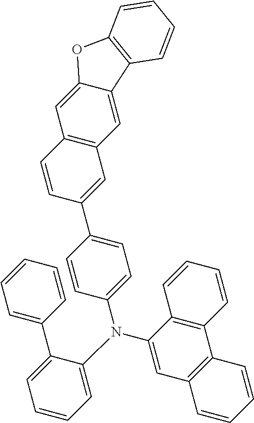 [SMILES: c1ccc(-c2ccccc2N(c2ccc(-c3ccc4cc5oc6ccccc6c5cc4c3)cc2)c2cc3ccccc3c3ccccc23)cc1]

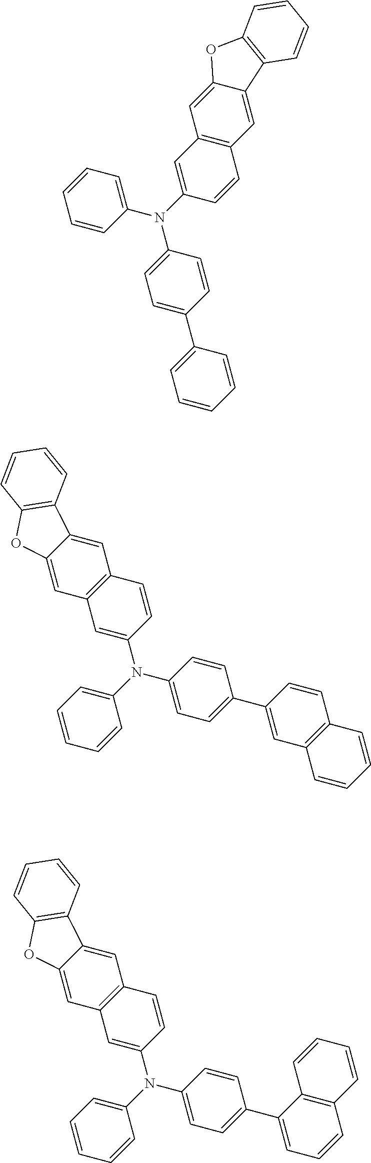 [SMILES: c1ccc(-c2ccc(N(c3ccccc3)c3ccc4cc5c(cc4c3)oc3ccccc35)cc2)cc1 ; c1ccc(N(c2ccc(-c3ccc4ccccc4c3)cc2)c2ccc3cc4c(cc3c2)oc2ccccc24)cc1 ; c1ccc(N(c2ccc(-c3cccc4ccccc34)cc2)c2ccc3cc4c(cc3c2)oc2ccccc24)cc1]

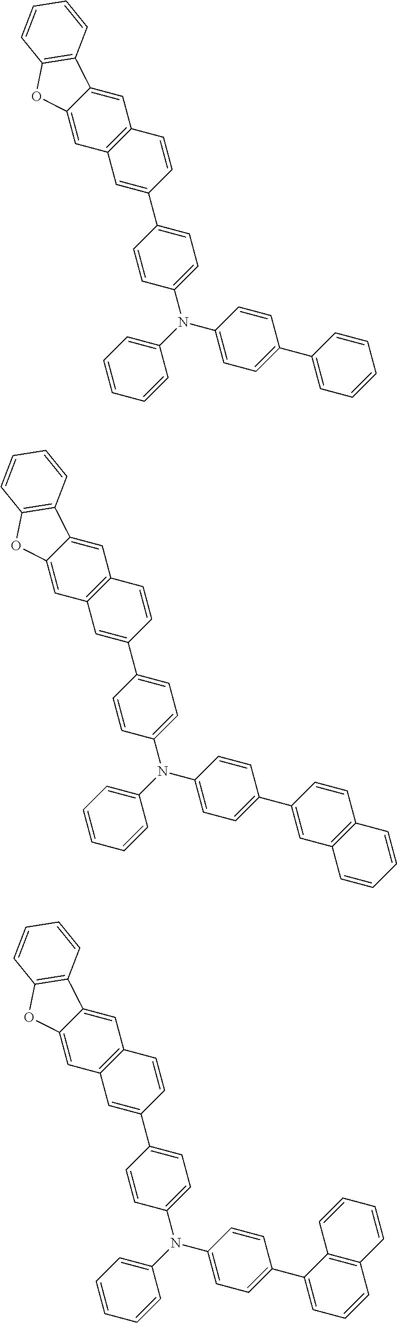 [SMILES: c1ccc(-c2ccc(N(c3ccccc3)c3ccc(-c4ccc5cc6c(cc5c4)oc4ccccc46)cc3)cc2)cc1 ; c1ccc(N(c2ccc(-c3ccc4ccccc4c3)cc2)c2ccc(-c3ccc4cc5c(cc4c3)oc3ccccc35)cc2)cc1 ; c1ccc(N(c2ccc(-c3ccc4cc5c(cc4c3)oc3ccccc35)cc2)c2ccc(-c3cccc4ccccc34)cc2)cc1]

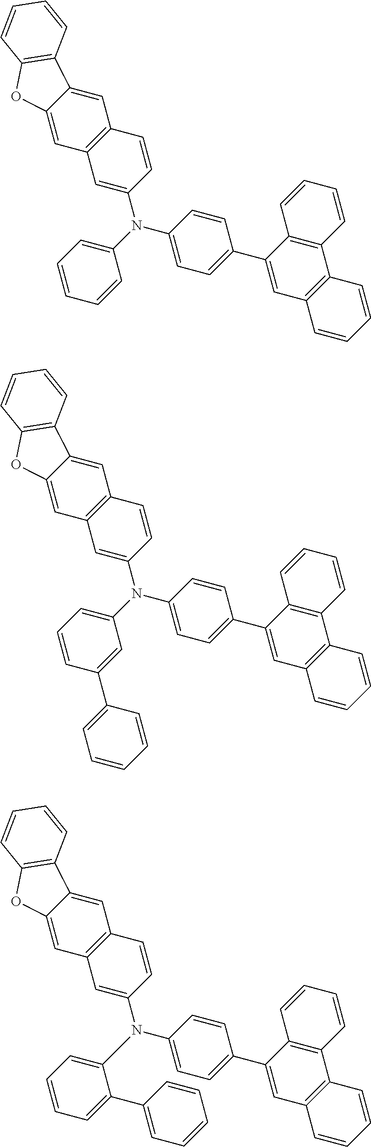 [SMILES: c1ccc(N(c2ccc(-c3cc4ccccc4c4ccccc34)cc2)c2ccc3cc4c(cc3c2)oc2ccccc24)cc1 ; c1ccc(-c2cccc(N(c3ccc(-c4cc5ccccc5c5ccccc45)cc3)c3ccc4cc5c(cc4c3)oc3ccccc35)c2)cc1 ; c1ccc(-c2ccccc2N(c2ccc(-c3cc4ccccc4c4ccccc34)cc2)c2ccc3cc4c(cc3c2)oc2ccccc24)cc1]

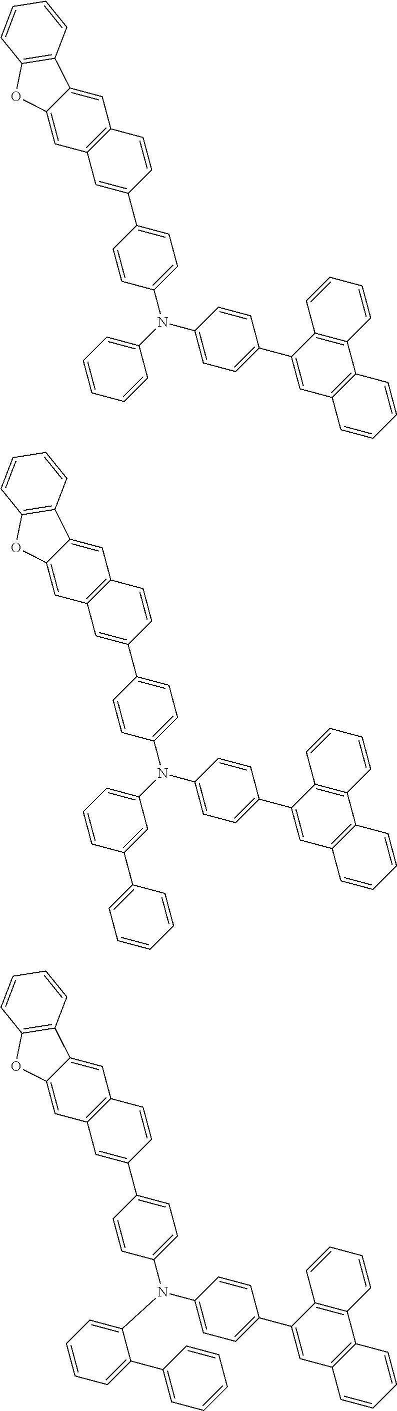 [SMILES: c1ccc(N(c2ccc(-c3ccc4cc5c(cc4c3)oc3ccccc35)cc2)c2ccc(-c3cc4ccccc4c4ccccc34)cc2)cc1 ; c1ccc(-c2cccc(N(c3ccc(-c4ccc5cc6c(cc5c4)oc4ccccc46)cc3)c3ccc(-c4cc5ccccc5c5ccccc45)cc3)c2)cc1 ; c1ccc(-c2ccccc2N(c2ccc(-c3ccc4cc5c(cc4c3)oc3ccccc35)cc2)c2ccc(-c3cc4ccccc4c4ccccc34)cc2)cc1]

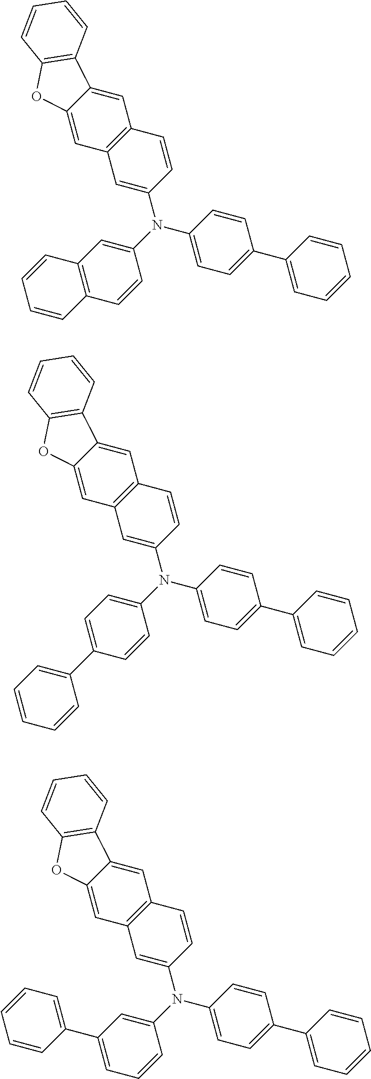

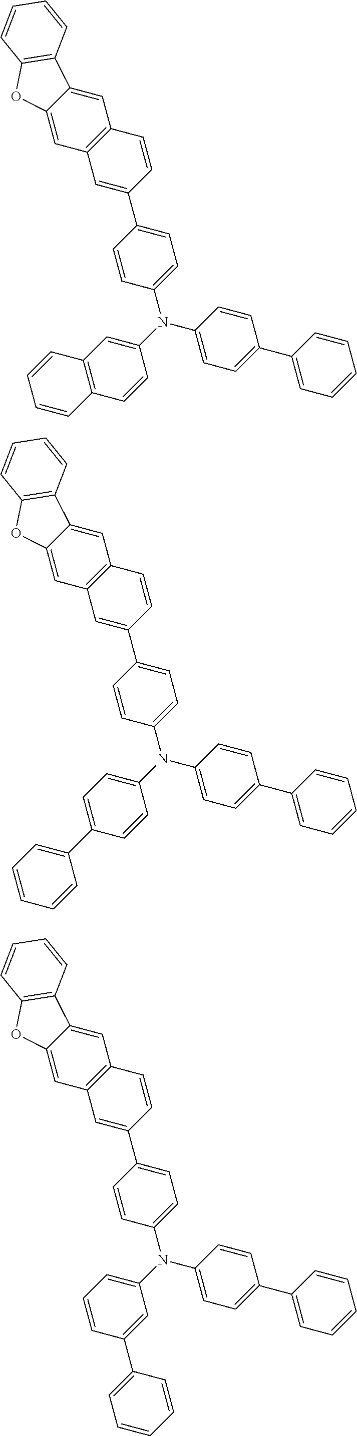

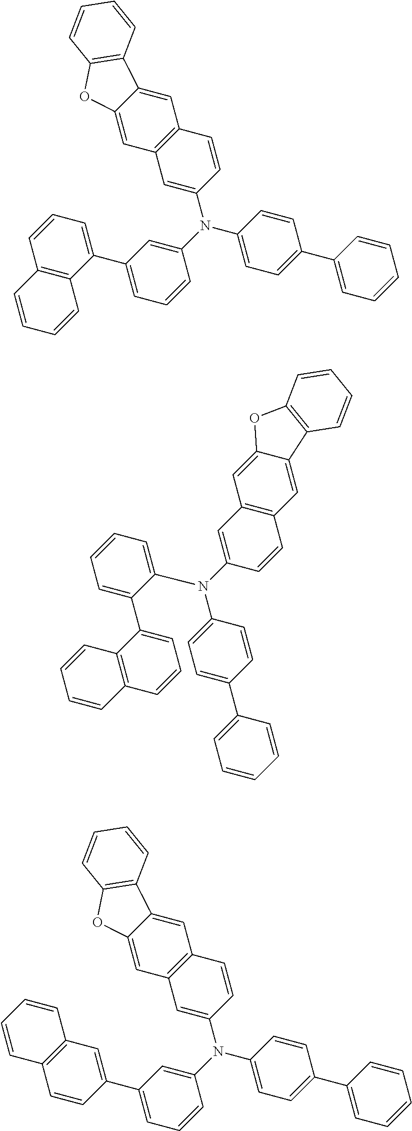 [SMILES: c1ccc(-c2ccc(N(c3cccc(-c4cccc5ccccc45)c3)c3ccc4cc5c(cc4c3)oc3ccccc35)cc2)cc1 ; c1ccc(-c2ccc(N(c3ccc4cc5c(cc4c3)oc3ccccc35)c3ccccc3-c3cccc4ccccc34)cc2)cc1 ; c1ccc(-c2ccc(N(c3cccc(-c4ccc5ccccc5c4)c3)c3ccc4cc5c(cc4c3)oc3ccccc35)cc2)cc1]

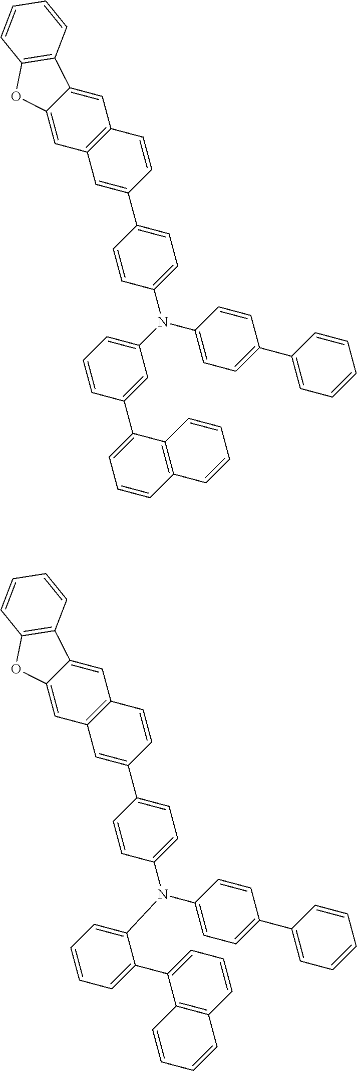 [SMILES: c1ccc(-c2ccc(N(c3ccc(-c4ccc5cc6c(cc5c4)oc4ccccc46)cc3)c3cccc(-c4cccc5ccccc45)c3)cc2)cc1 ; c1ccc(-c2ccc(N(c3ccc(-c4ccc5cc6c(cc5c4)oc4ccccc46)cc3)c3ccccc3-c3cccc4ccccc34)cc2)cc1]

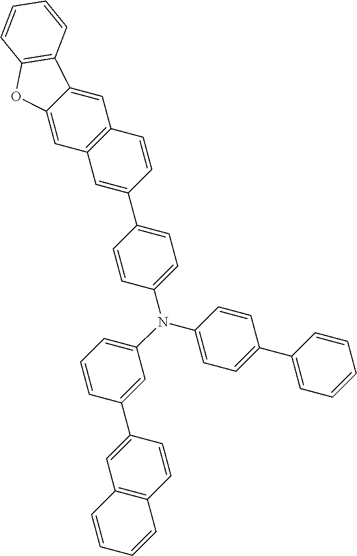 [SMILES: c1ccc(-c2ccc(N(c3ccc(-c4ccc5cc6c(cc5c4)oc4ccccc46)cc3)c3cccc(-c4ccc5ccccc5c4)c3)cc2)cc1]

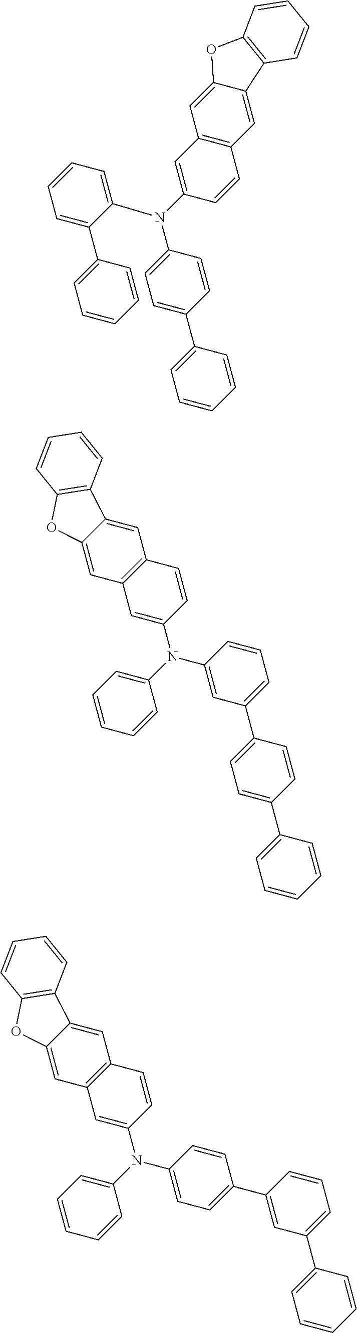 [SMILES: c1ccc(-c2ccc(N(c3ccc4cc5c(cc4c3)oc3ccccc35)c3ccccc3-c3ccccc3)cc2)cc1 ; c1ccc(-c2ccc(-c3cccc(N(c4ccccc4)c4ccc5cc6c(cc5c4)oc4ccccc46)c3)cc2)cc1 ; c1ccc(-c2cccc(-c3ccc(N(c4ccccc4)c4ccc5cc6c(cc5c4)oc4ccccc46)cc3)c2)cc1]

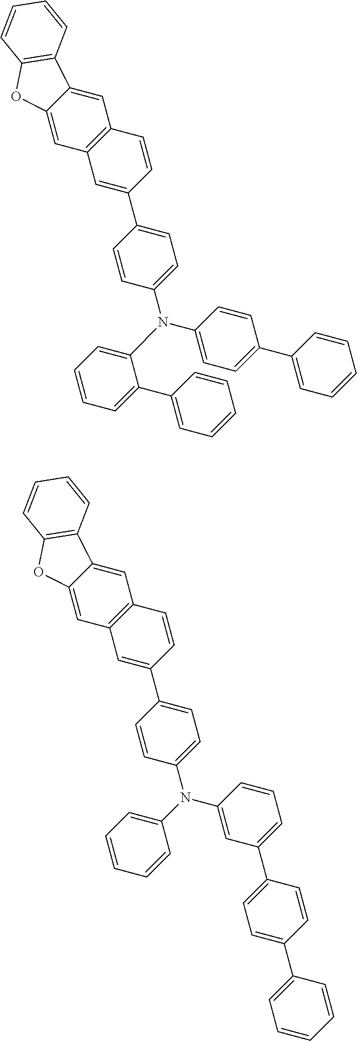 [SMILES: c1ccc(-c2ccc(N(c3ccc(-c4ccc5cc6c(cc5c4)oc4ccccc46)cc3)c3ccccc3-c3ccccc3)cc2)cc1 ; c1ccc(-c2ccc(-c3cccc(N(c4ccccc4)c4ccc(-c5ccc6cc7c(cc6c5)oc5ccccc57)cc4)c3)cc2)cc1]

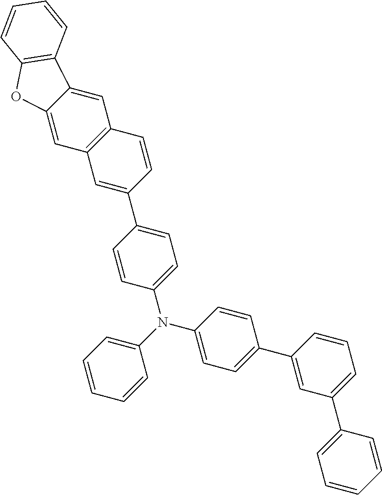 [SMILES: c1ccc(-c2cccc(-c3ccc(N(c4ccccc4)c4ccc(-c5ccc6cc7c(cc6c5)oc5ccccc57)cc4)cc3)c2)cc1]

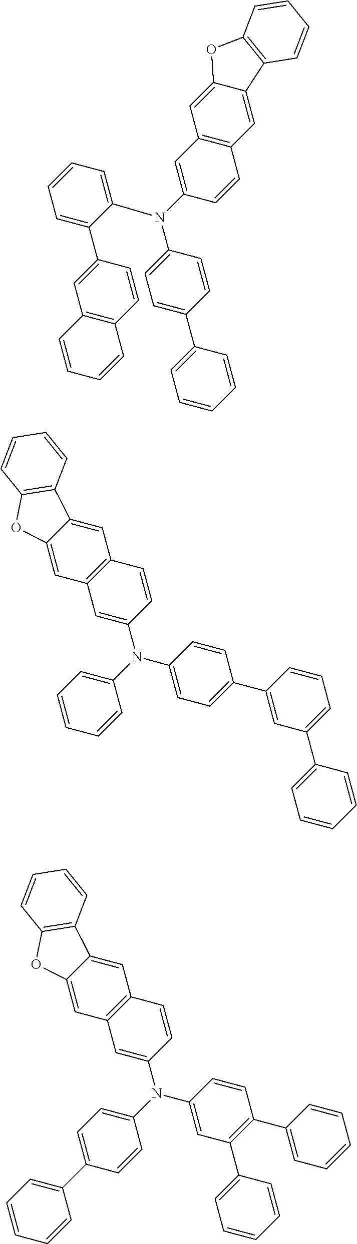 [SMILES: c1ccc(-c2ccc(N(c3ccc4cc5c(cc4c3)oc3ccccc35)c3ccccc3-c3ccc4ccccc4c3)cc2)cc1 ; c1ccc(-c2cccc(-c3ccc(N(c4ccccc4)c4ccc5cc6c(cc5c4)oc4ccccc46)cc3)c2)cc1 ; c1ccc(-c2ccc(N(c3ccc(-c4ccccc4)c(-c4ccccc4)c3)c3ccc4cc5c(cc4c3)oc3ccccc35)cc2)cc1]

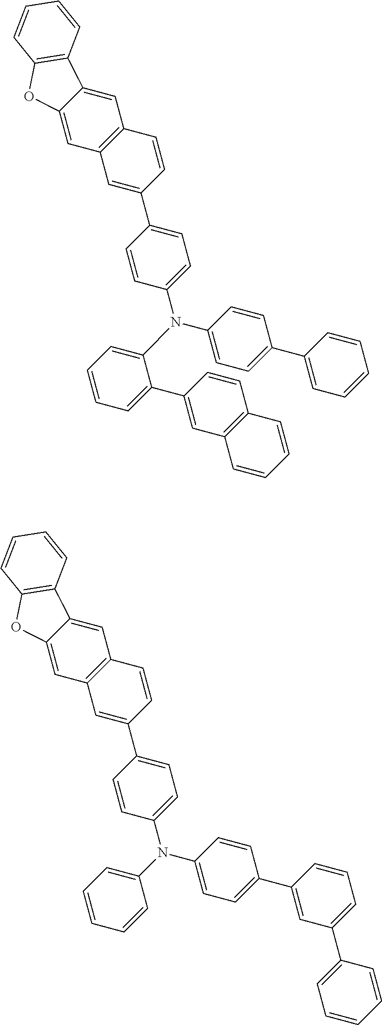 [SMILES: c1ccc(-c2ccc(N(c3ccc(-c4ccc5cc6c(cc5c4)oc4ccccc46)cc3)c3ccccc3-c3ccc4ccccc4c3)cc2)cc1 ; c1ccc(-c2cccc(-c3ccc(N(c4ccccc4)c4ccc(-c5ccc6cc7c(cc6c5)oc5ccccc57)cc4)cc3)c2)cc1]

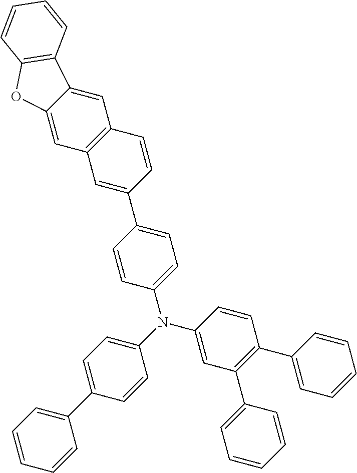 [SMILES: c1ccc(-c2ccc(N(c3ccc(-c4ccc5cc6c(cc5c4)oc4ccccc46)cc3)c3ccc(-c4ccccc4)c(-c4ccccc4)c3)cc2)cc1]

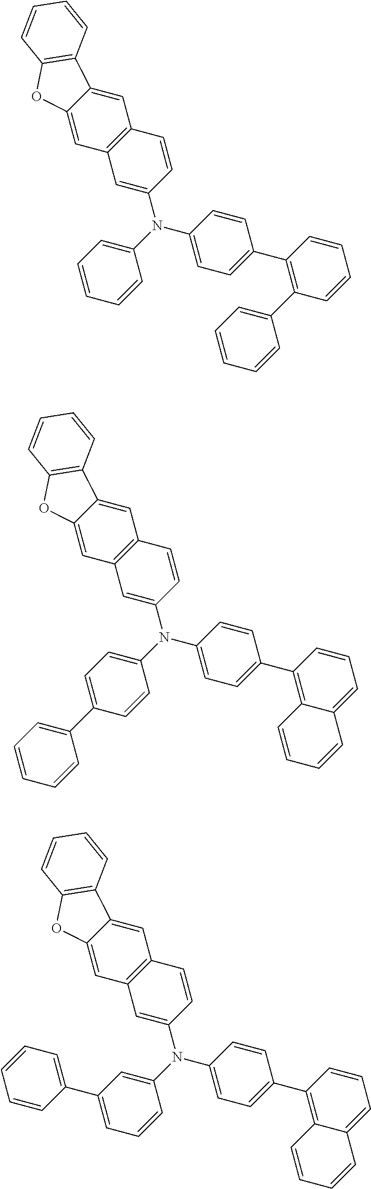 [SMILES: c1ccc(-c2ccccc2-c2ccc(N(c3ccccc3)c3ccc4cc5c(cc4c3)oc3ccccc35)cc2)cc1 ; c1ccc(-c2ccc(N(c3ccc(-c4cccc5ccccc45)cc3)c3ccc4cc5c(cc4c3)oc3ccccc35)cc2)cc1 ; c1ccc(-c2cccc(N(c3ccc(-c4cccc5ccccc45)cc3)c3ccc4cc5c(cc4c3)oc3ccccc35)c2)cc1]

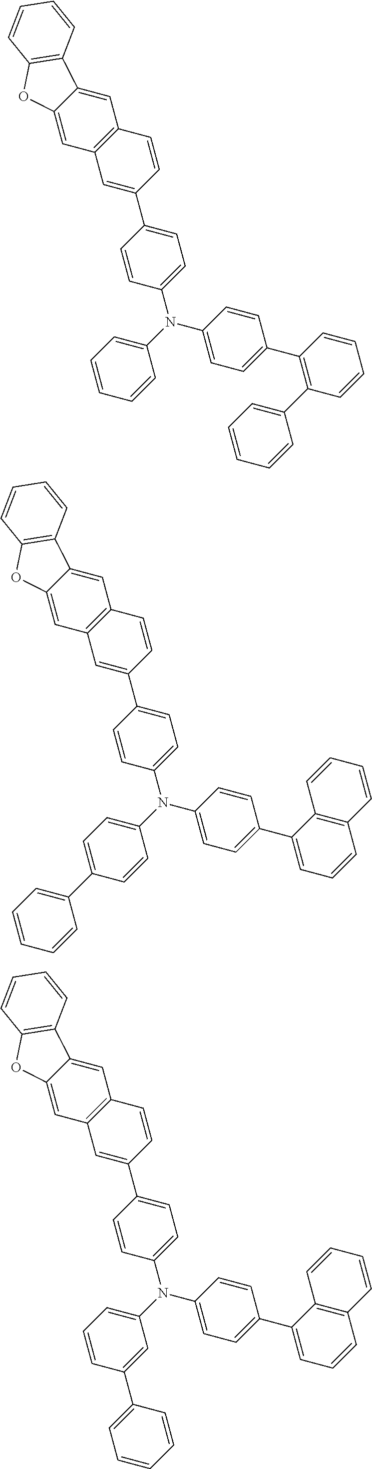

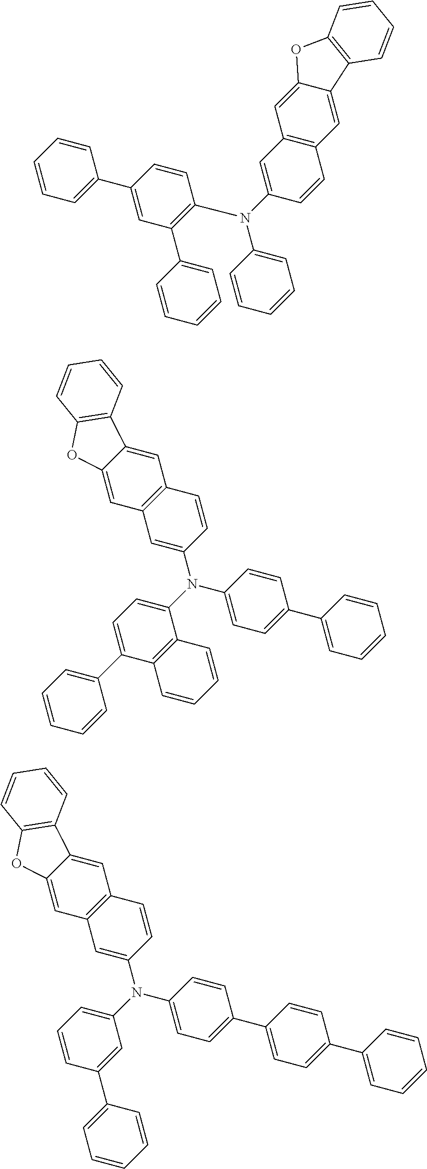

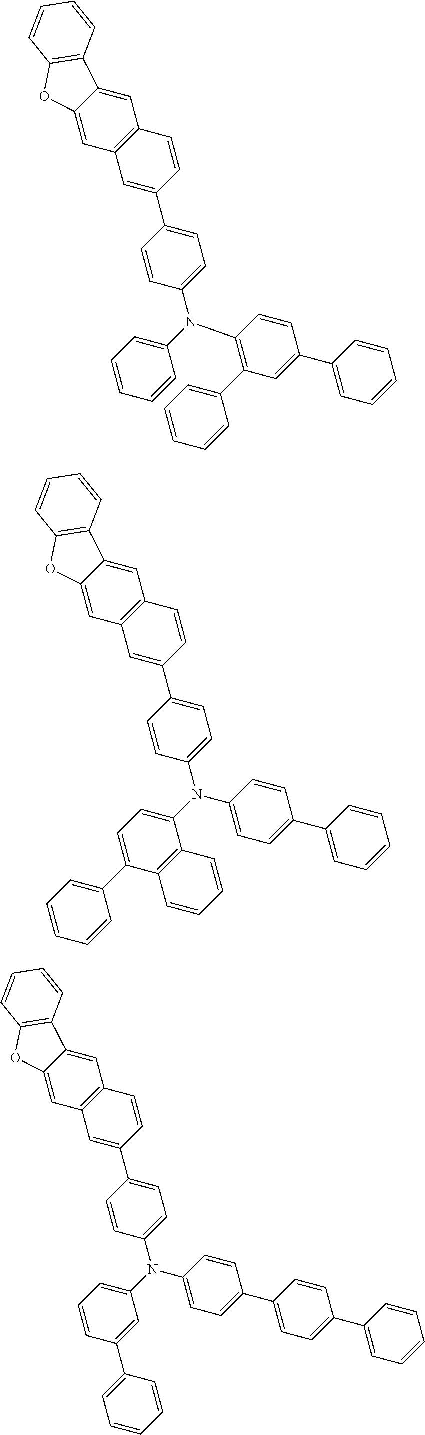

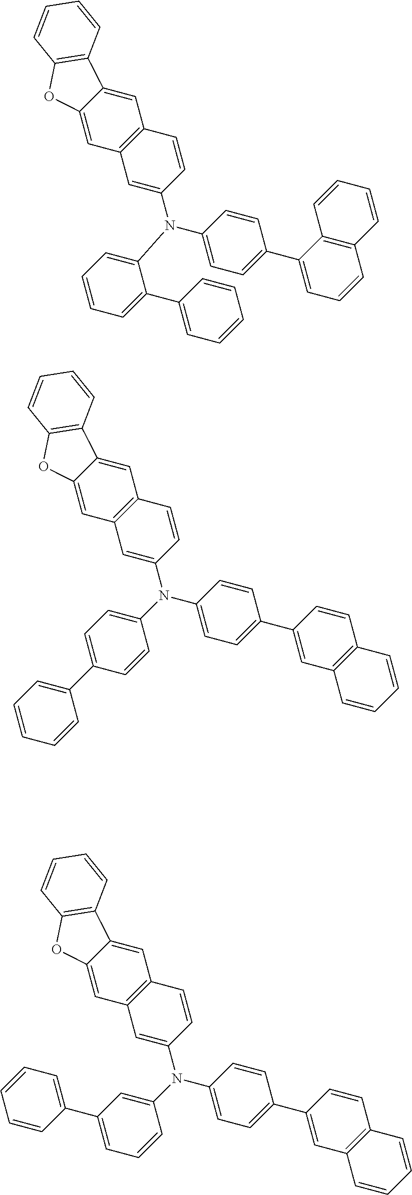 [SMILES: c1ccc(-c2ccccc2N(c2ccc(-c3cccc4ccccc34)cc2)c2ccc3cc4c(cc3c2)oc2ccccc24)cc1 ; c1ccc(-c2ccc(N(c3ccc(-c4ccc5ccccc5c4)cc3)c3ccc4cc5c(cc4c3)oc3ccccc35)cc2)cc1 ; c1ccc(-c2cccc(N(c3ccc(-c4ccc5ccccc5c4)cc3)c3ccc4cc5c(cc4c3)oc3ccccc35)c2)cc1]

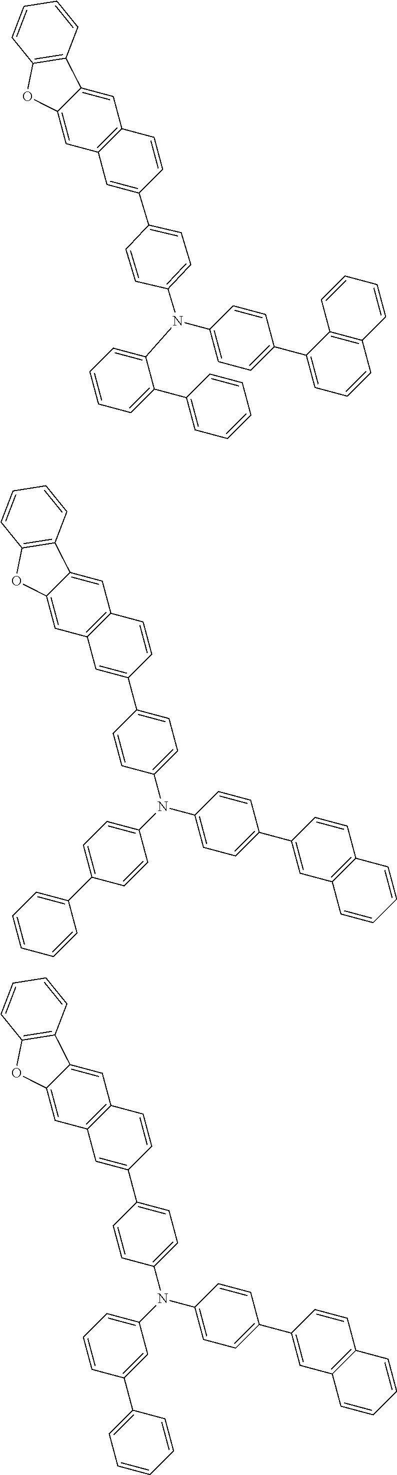

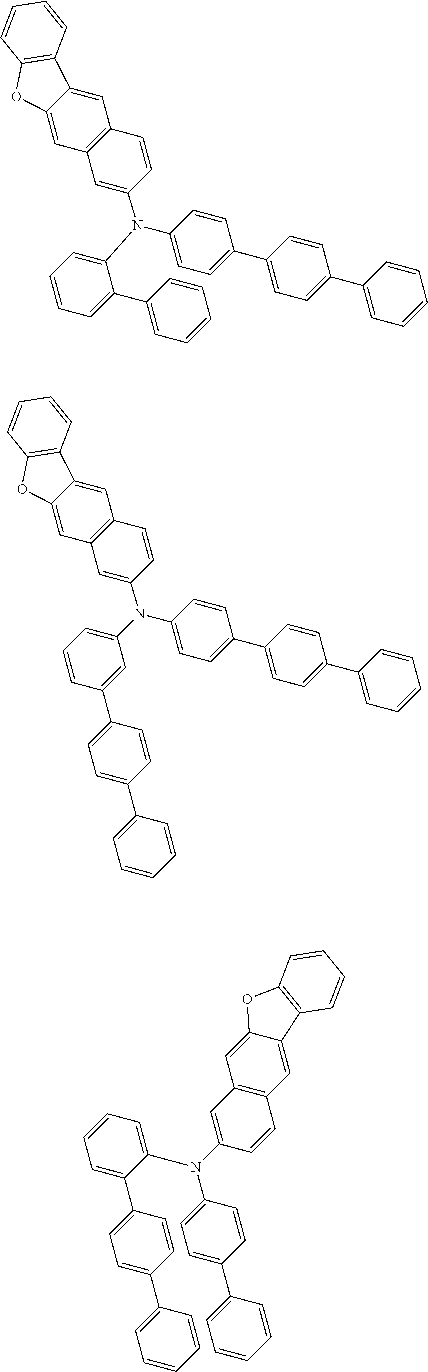 [SMILES: c1ccc(-c2ccc(-c3ccc(N(c4ccc5cc6c(cc5c4)oc4ccccc46)c4ccccc4-c4ccccc4)cc3)cc2)cc1 ; c1ccc(-c2ccc(-c3ccc(N(c4cccc(-c5ccc(-c6ccccc6)cc5)c4)c4ccc5cc6c(cc5c4)oc4ccccc46)cc3)cc2)cc1 ; c1ccc(-c2ccc(-c3ccccc3N(c3ccc(-c4ccccc4)cc3)c3ccc4cc5c(cc4c3)oc3ccccc35)cc2)cc1]

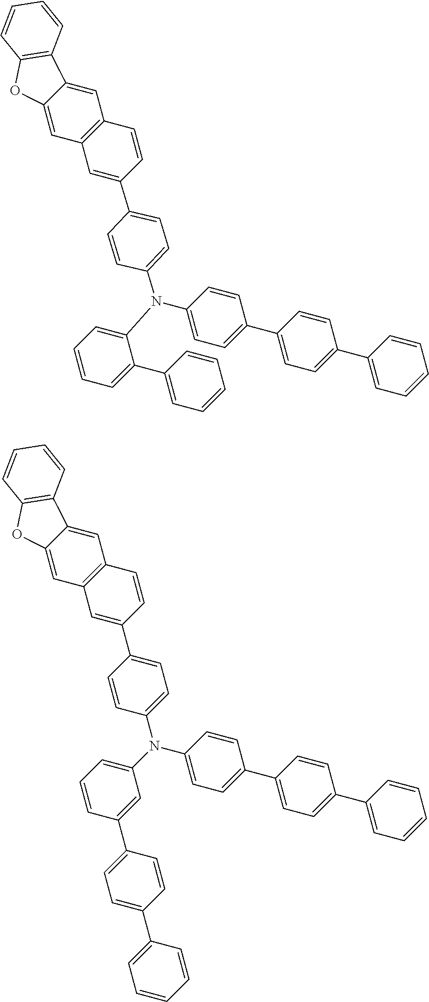 [SMILES: c1ccc(-c2ccc(-c3ccc(N(c4ccc(-c5ccc6cc7c(cc6c5)oc5ccccc57)cc4)c4ccccc4-c4ccccc4)cc3)cc2)cc1 ; c1ccc(-c2ccc(-c3ccc(N(c4ccc(-c5ccc6cc7c(cc6c5)oc5ccccc57)cc4)c4cccc(-c5ccc(-c6ccccc6)cc5)c4)cc3)cc2)cc1]

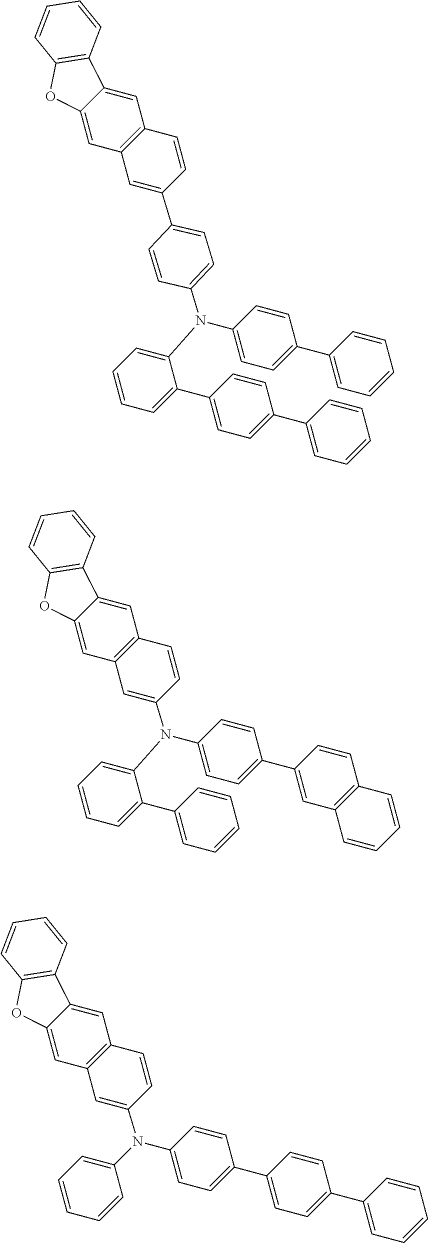 [SMILES: c1ccc(-c2ccc(-c3ccccc3N(c3ccc(-c4ccccc4)cc3)c3ccc(-c4ccc5cc6c(cc5c4)oc4ccccc46)cc3)cc2)cc1 ; c1ccc(-c2ccccc2N(c2ccc(-c3ccc4ccccc4c3)cc2)c2ccc3cc4c(cc3c2)oc2ccccc24)cc1 ; c1ccc(-c2ccc(-c3ccc(N(c4ccccc4)c4ccc5cc6c(cc5c4)oc4ccccc46)cc3)cc2)cc1]

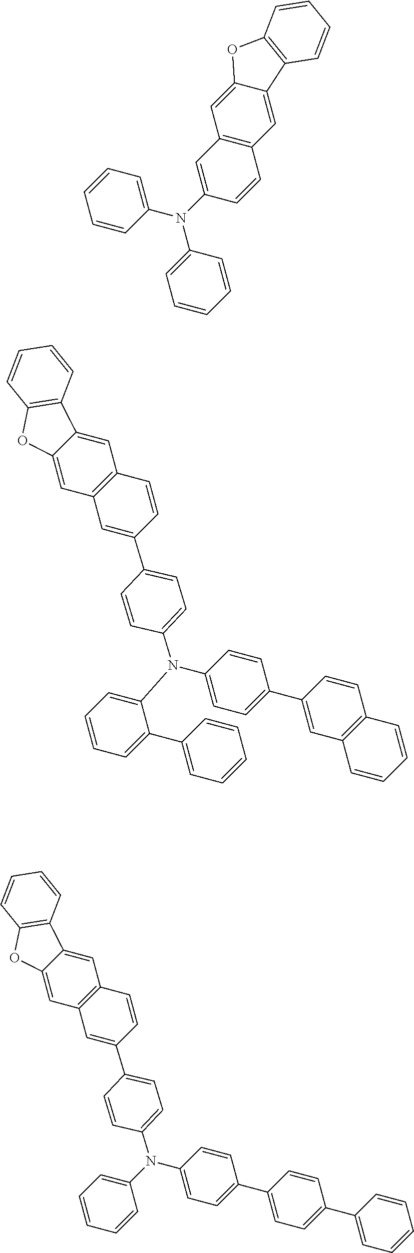 [SMILES: c1ccc(N(c2ccccc2)c2ccc3cc4c(cc3c2)oc2ccccc24)cc1 ; c1ccc(-c2ccccc2N(c2ccc(-c3ccc4ccccc4c3)cc2)c2ccc(-c3ccc4cc5c(cc4c3)oc3ccccc35)cc2)cc1 ; c1ccc(-c2ccc(-c3ccc(N(c4ccccc4)c4ccc(-c5ccc6cc7c(cc6c5)oc5ccccc57)cc4)cc3)cc2)cc1]

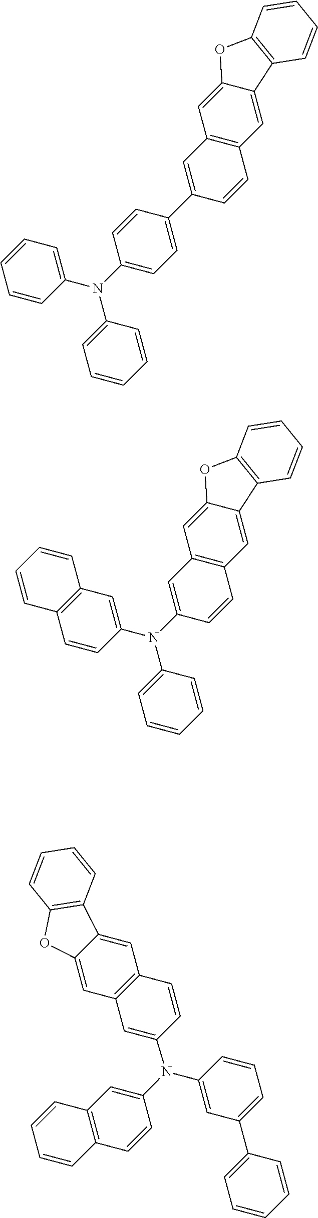 [SMILES: c1ccc(N(c2ccccc2)c2ccc(-c3ccc4cc5c(cc4c3)oc3ccccc35)cc2)cc1 ; c1ccc(N(c2ccc3ccccc3c2)c2ccc3cc4c(cc3c2)oc2ccccc24)cc1 ; c1ccc(-c2cccc(N(c3ccc4ccccc4c3)c3ccc4cc5c(cc4c3)oc3ccccc35)c2)cc1]

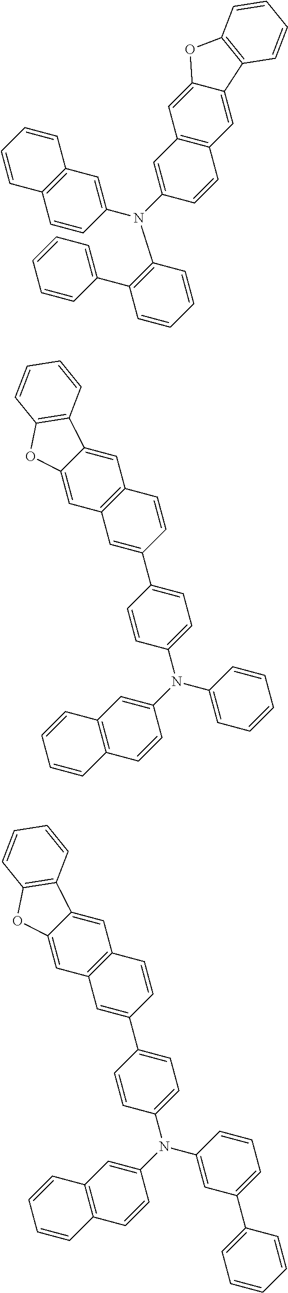 [SMILES: c1ccc(-c2ccccc2N(c2ccc3ccccc3c2)c2ccc3cc4c(cc3c2)oc2ccccc24)cc1 ; c1ccc(N(c2ccc(-c3ccc4cc5c(cc4c3)oc3ccccc35)cc2)c2ccc3ccccc3c2)cc1 ; c1ccc(-c2cccc(N(c3ccc(-c4ccc5cc6c(cc5c4)oc4ccccc46)cc3)c3ccc4ccccc4c3)c2)cc1]

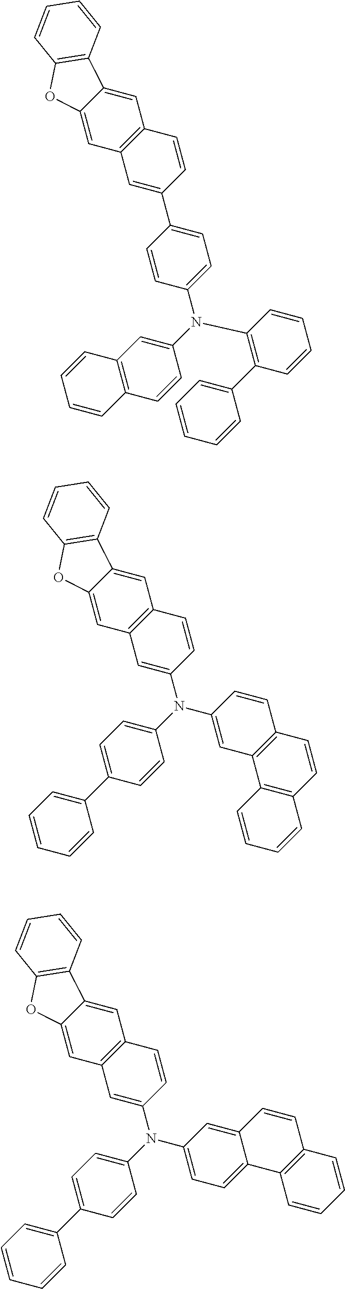 [SMILES: c1ccc(-c2ccccc2N(c2ccc(-c3ccc4cc5c(cc4c3)oc3ccccc35)cc2)c2ccc3ccccc3c2)cc1 ; c1ccc(-c2ccc(N(c3ccc4cc5c(cc4c3)oc3ccccc35)c3ccc4ccc5ccccc5c4c3)cc2)cc1 ; c1ccc(-c2ccc(N(c3ccc4cc5c(cc4c3)oc3ccccc35)c3ccc4c(ccc5ccccc54)c3)cc2)cc1]

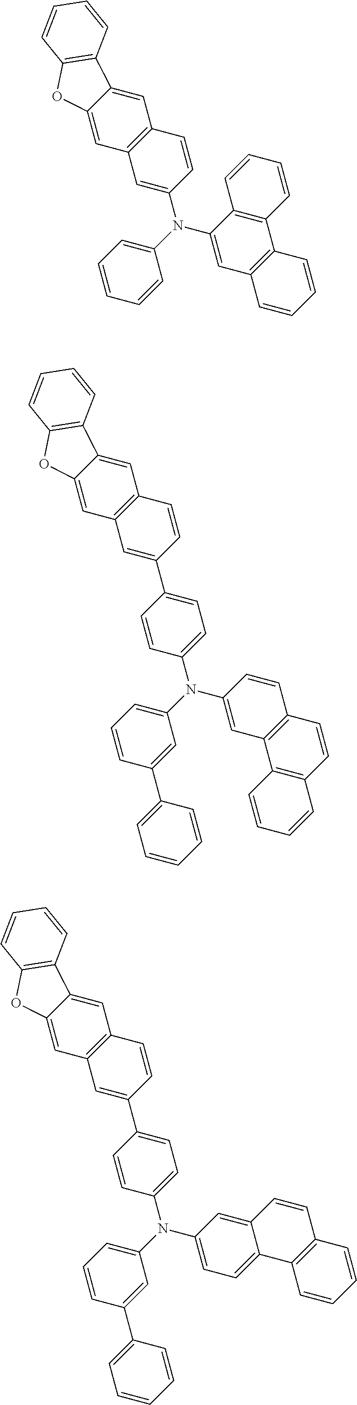 [SMILES: c1ccc(N(c2ccc3cc4c(cc3c2)oc2ccccc24)c2cc3ccccc3c3ccccc23)cc1 ; c1ccc(-c2cccc(N(c3ccc(-c4ccc5cc6c(cc5c4)oc4ccccc46)cc3)c3ccc4ccc5ccccc5c4c3)c2)cc1 ; c1ccc(-c2cccc(N(c3ccc(-c4ccc5cc6c(cc5c4)oc4ccccc46)cc3)c3ccc4c(ccc5ccccc54)c3)c2)cc1]

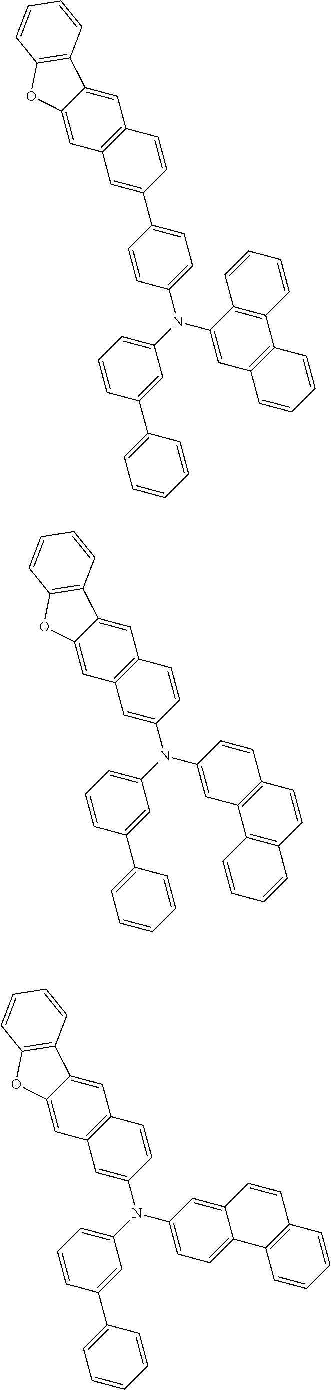 [SMILES: c1ccc(-c2cccc(N(c3ccc(-c4ccc5cc6c(cc5c4)oc4ccccc46)cc3)c3cc4ccccc4c4ccccc34)c2)cc1 ; c1ccc(-c2cccc(N(c3ccc4cc5c(cc4c3)oc3ccccc35)c3ccc4ccc5ccccc5c4c3)c2)cc1 ; c1ccc(-c2cccc(N(c3ccc4cc5c(cc4c3)oc3ccccc35)c3ccc4c(ccc5ccccc54)c3)c2)cc1]

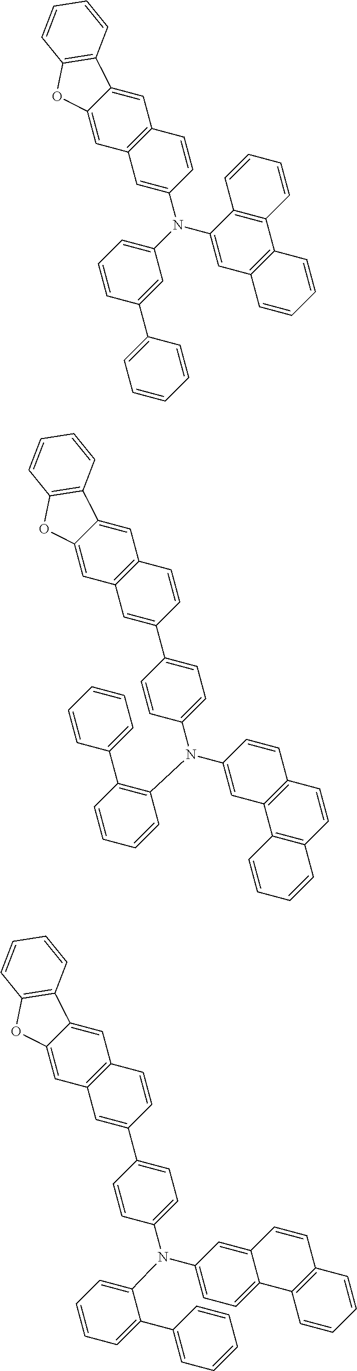 [SMILES: c1ccc(-c2cccc(N(c3ccc4cc5c(cc4c3)oc3ccccc35)c3cc4ccccc4c4ccccc34)c2)cc1 ; c1ccc(-c2ccccc2N(c2ccc(-c3ccc4cc5c(cc4c3)oc3ccccc35)cc2)c2ccc3ccc4ccccc4c3c2)cc1 ; c1ccc(-c2ccccc2N(c2ccc(-c3ccc4cc5c(cc4c3)oc3ccccc35)cc2)c2ccc3c(ccc4ccccc43)c2)cc1]

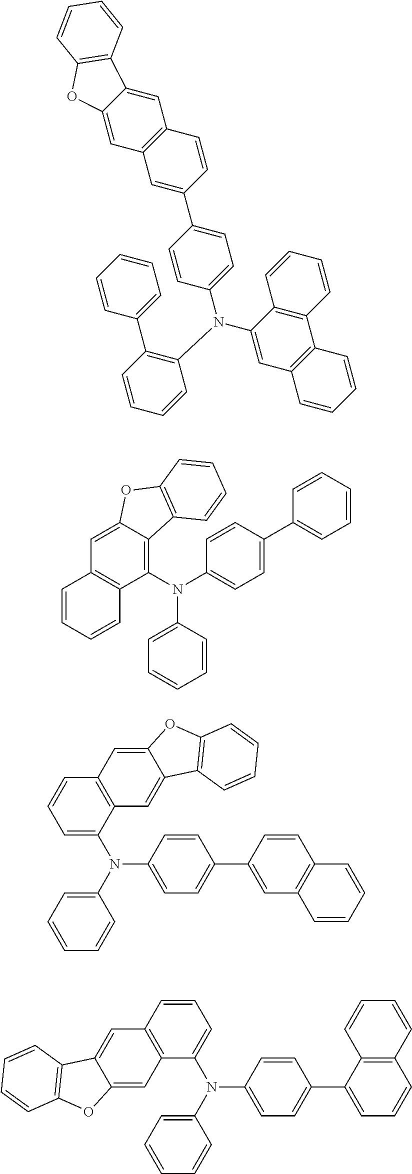

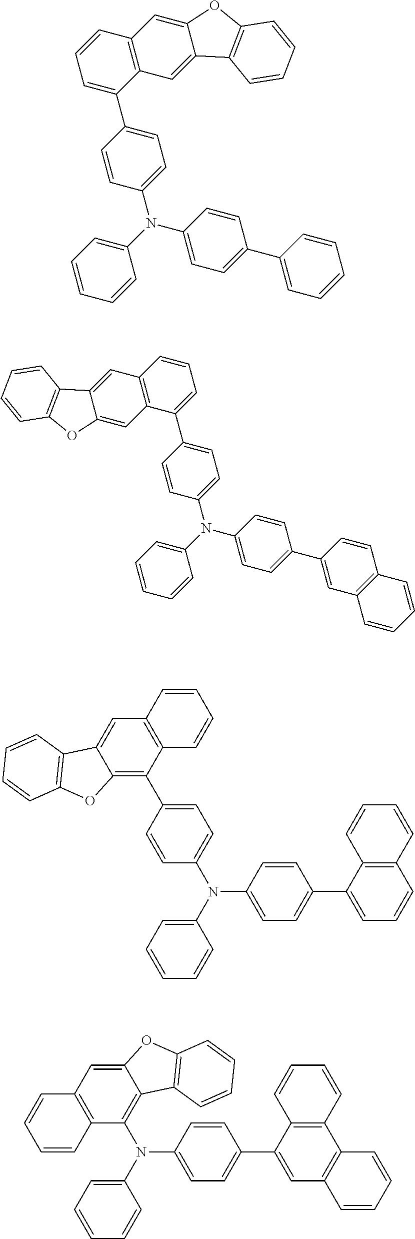 [SMILES: c1ccc(-c2ccc(N(c3ccccc3)c3ccc(-c4cccc5cc6oc7ccccc7c6cc45)cc3)cc2)cc1 ; c1ccc(N(c2ccc(-c3ccc4ccccc4c3)cc2)c2ccc(-c3cccc4cc5c(cc34)oc3ccccc35)cc2)cc1 ; c1ccc(N(c2ccc(-c3cccc4ccccc34)cc2)c2ccc(-c3c4ccccc4cc4c3oc3ccccc34)cc2)cc1 ; c1ccc(N(c2ccc(-c3cc4ccccc4c4ccccc34)cc2)c2c3ccccc3cc3oc4ccccc4c23)cc1]

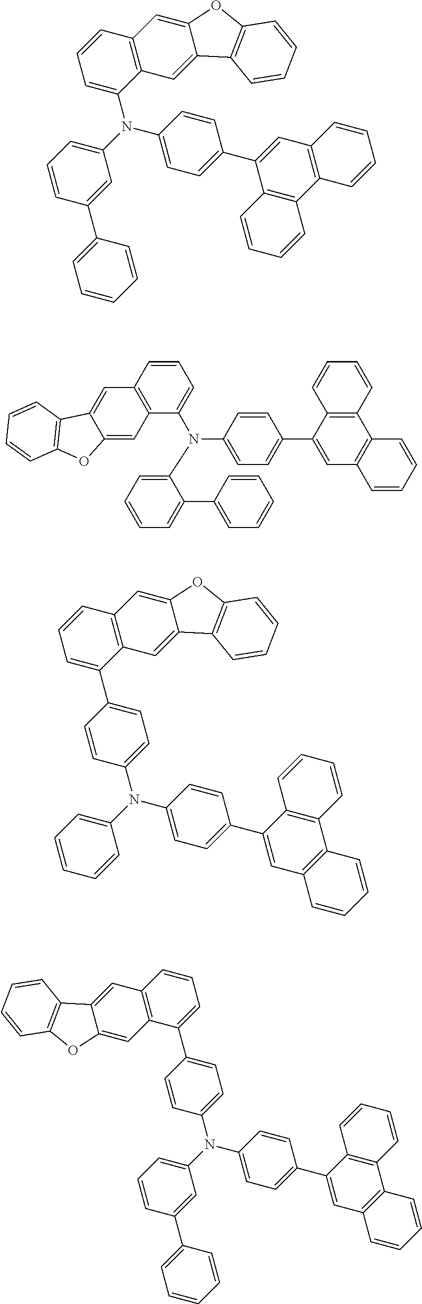 [SMILES: c1ccc(-c2cccc(N(c3ccc(-c4cc5ccccc5c5ccccc45)cc3)c3cccc4cc5oc6ccccc6c5cc34)c2)cc1 ; c1ccc(-c2ccccc2N(c2ccc(-c3cc4ccccc4c4ccccc34)cc2)c2cccc3cc4c(cc23)oc2ccccc24)cc1 ; c1ccc(N(c2ccc(-c3cccc4cc5oc6ccccc6c5cc34)cc2)c2ccc(-c3cc4ccccc4c4ccccc34)cc2)cc1 ; c1ccc(-c2cccc(N(c3ccc(-c4cccc5cc6c(cc45)oc4ccccc46)cc3)c3ccc(-c4cc5ccccc5c5ccccc45)cc3)c2)cc1]

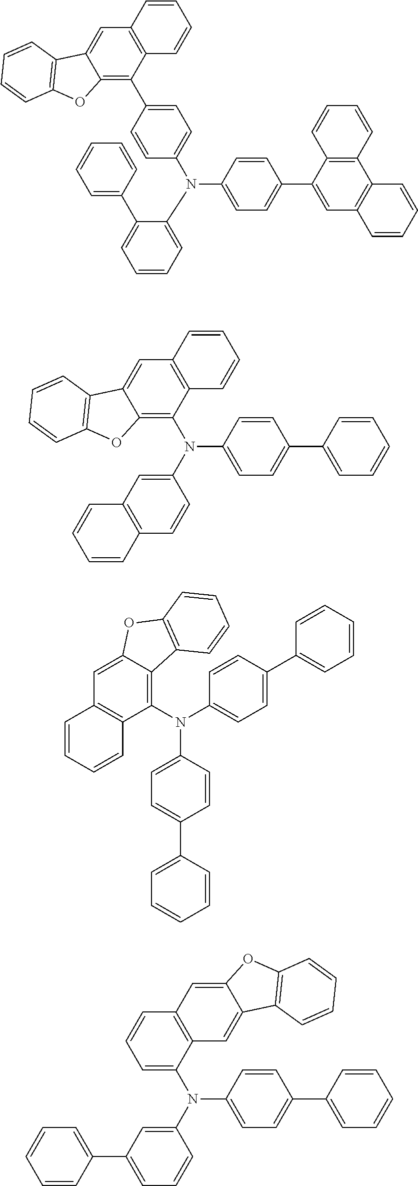 [SMILES: c1ccc(-c2ccccc2N(c2ccc(-c3cc4ccccc4c4ccccc34)cc2)c2ccc(-c3c4ccccc4cc4c3oc3ccccc34)cc2)cc1 ; c1ccc(-c2ccc(N(c3ccc4ccccc4c3)c3c4ccccc4cc4c3oc3ccccc34)cc2)cc1 ; c1ccc(-c2ccc(N(c3ccc(-c4ccccc4)cc3)c3c4ccccc4cc4oc5ccccc5c34)cc2)cc1 ; c1ccc(-c2ccc(N(c3cccc(-c4ccccc4)c3)c3cccc4cc5oc6ccccc6c5cc34)cc2)cc1]

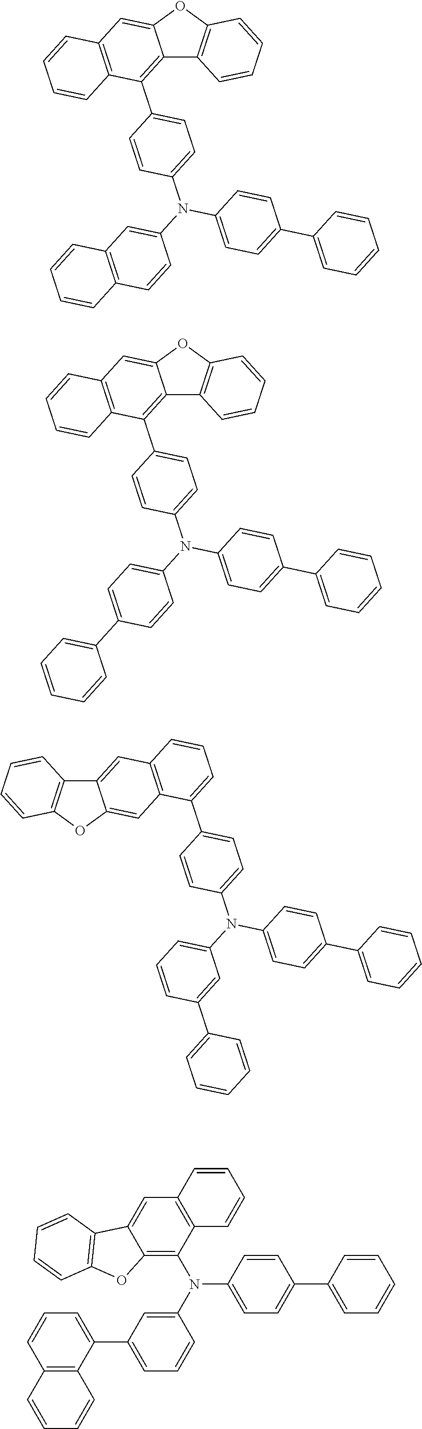 [SMILES: c1ccc(-c2ccc(N(c3ccc(-c4c5ccccc5cc5oc6ccccc6c45)cc3)c3ccc4ccccc4c3)cc2)cc1 ; c1ccc(-c2ccc(N(c3ccc(-c4ccccc4)cc3)c3ccc(-c4c5ccccc5cc5oc6ccccc6c45)cc3)cc2)cc1 ; c1ccc(-c2ccc(N(c3ccc(-c4cccc5cc6c(cc45)oc4ccccc46)cc3)c3cccc(-c4ccccc4)c3)cc2)cc1 ; c1ccc(-c2ccc(N(c3cccc(-c4cccc5ccccc45)c3)c3c4ccccc4cc4c3oc3ccccc34)cc2)cc1]

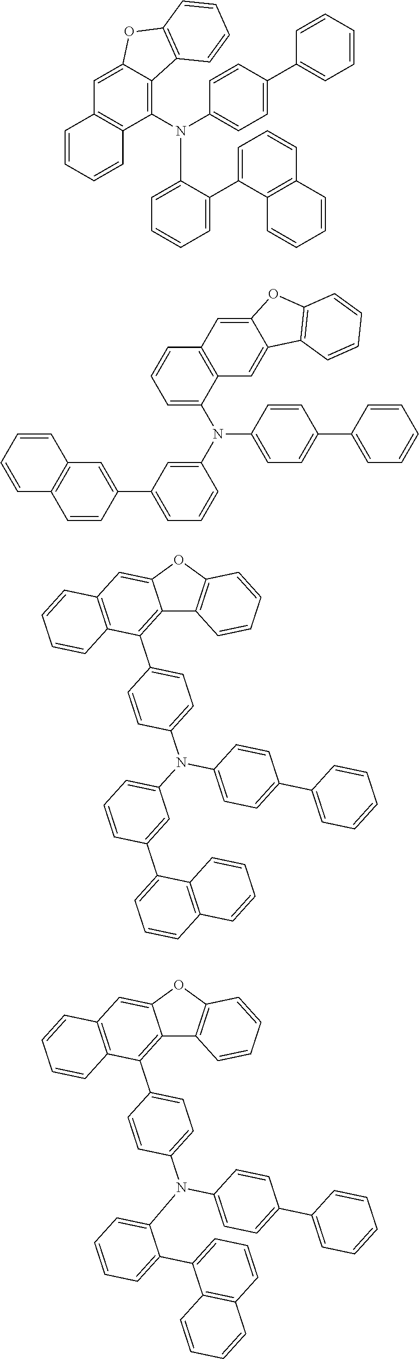

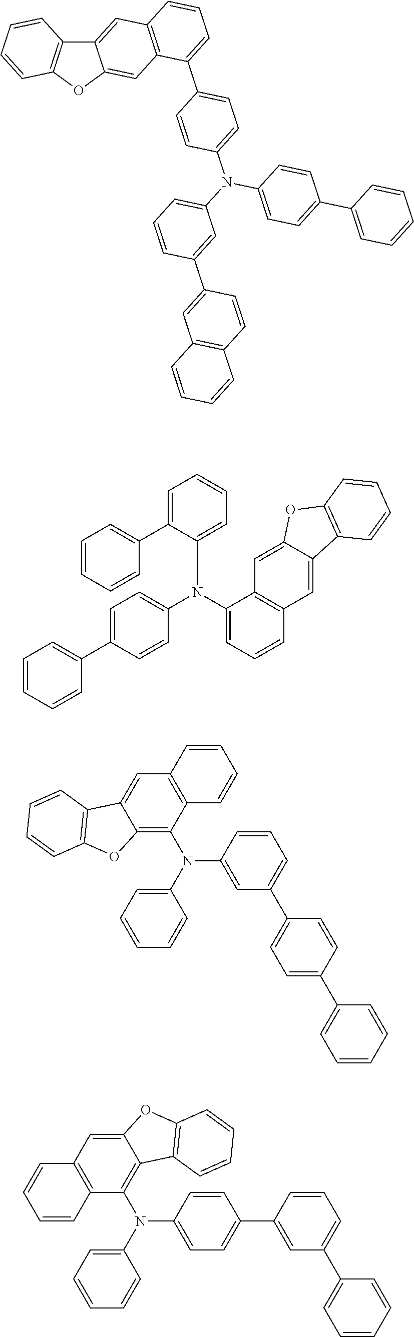 [SMILES: c1ccc(-c2ccc(N(c3ccc(-c4cccc5cc6c(cc45)oc4ccccc46)cc3)c3cccc(-c4ccc5ccccc5c4)c3)cc2)cc1 ; c1ccc(-c2ccc(N(c3ccccc3-c3ccccc3)c3cccc4cc5c(cc34)oc3ccccc35)cc2)cc1 ; c1ccc(-c2ccc(-c3cccc(N(c4ccccc4)c4c5ccccc5cc5c4oc4ccccc45)c3)cc2)cc1 ; c1ccc(-c2cccc(-c3ccc(N(c4ccccc4)c4c5ccccc5cc5oc6ccccc6c45)cc3)c2)cc1]

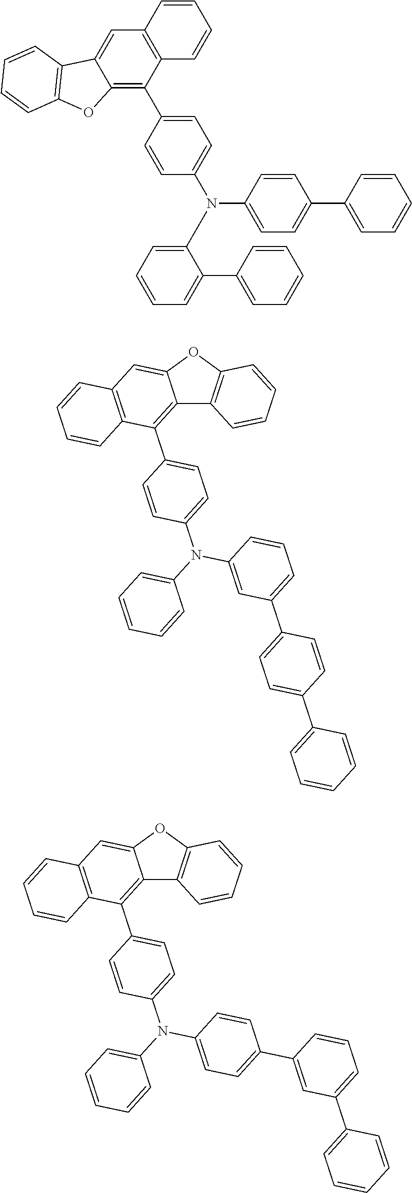 [SMILES: c1ccc(-c2ccc(N(c3ccc(-c4c5ccccc5cc5c4oc4ccccc45)cc3)c3ccccc3-c3ccccc3)cc2)cc1 ; c1ccc(-c2ccc(-c3cccc(N(c4ccccc4)c4ccc(-c5c6ccccc6cc6oc7ccccc7c56)cc4)c3)cc2)cc1 ; c1ccc(-c2cccc(-c3ccc(N(c4ccccc4)c4ccc(-c5c6ccccc6cc6oc7ccccc7c56)cc4)cc3)c2)cc1]

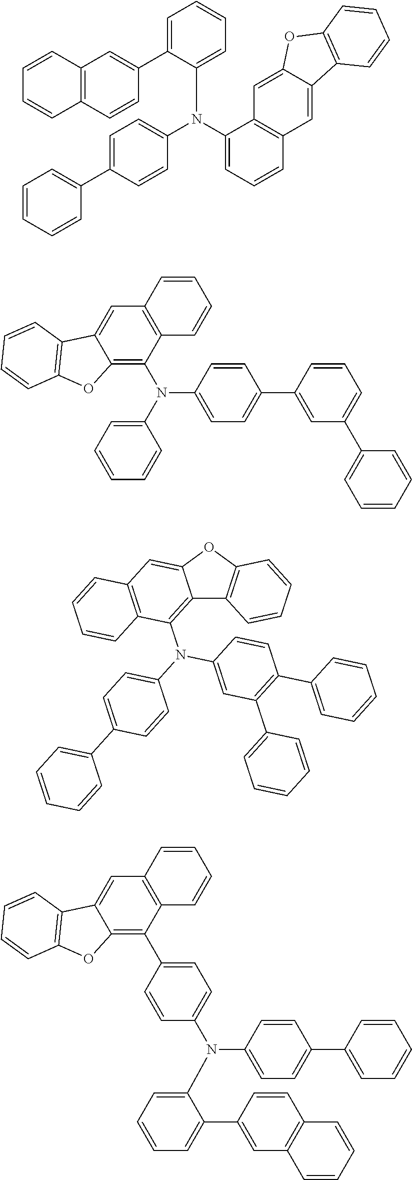 [SMILES: c1ccc(-c2ccc(N(c3ccccc3-c3ccc4ccccc4c3)c3cccc4cc5c(cc34)oc3ccccc35)cc2)cc1 ; c1ccc(-c2cccc(-c3ccc(N(c4ccccc4)c4c5ccccc5cc5c4oc4ccccc45)cc3)c2)cc1 ; c1ccc(-c2ccc(N(c3ccc(-c4ccccc4)c(-c4ccccc4)c3)c3c4ccccc4cc4oc5ccccc5c34)cc2)cc1 ; c1ccc(-c2ccc(N(c3ccc(-c4c5ccccc5cc5c4oc4ccccc45)cc3)c3ccccc3-c3ccc4ccccc4c3)cc2)cc1]

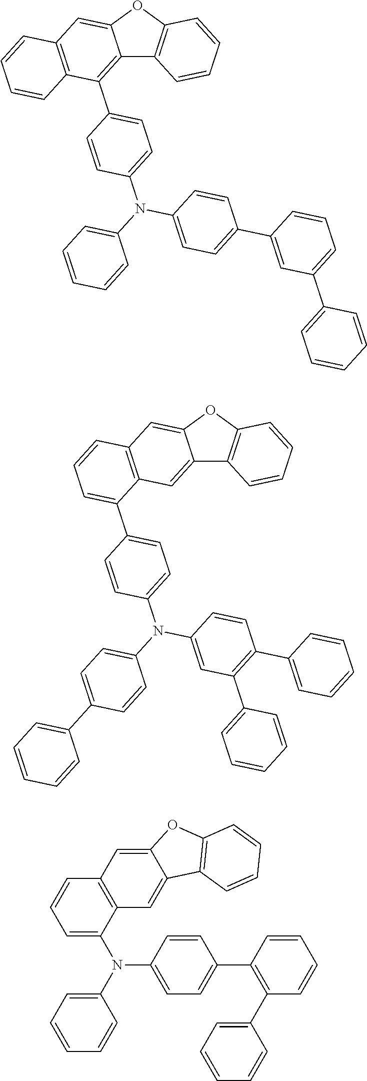 [SMILES: c1ccc(-c2cccc(-c3ccc(N(c4ccccc4)c4ccc(-c5c6ccccc6cc6oc7ccccc7c56)cc4)cc3)c2)cc1 ; c1ccc(-c2ccc(N(c3ccc(-c4cccc5cc6oc7ccccc7c6cc45)cc3)c3ccc(-c4ccccc4)c(-c4ccccc4)c3)cc2)cc1 ; c1ccc(-c2ccccc2-c2ccc(N(c3ccccc3)c3cccc4cc5oc6ccccc6c5cc34)cc2)cc1]

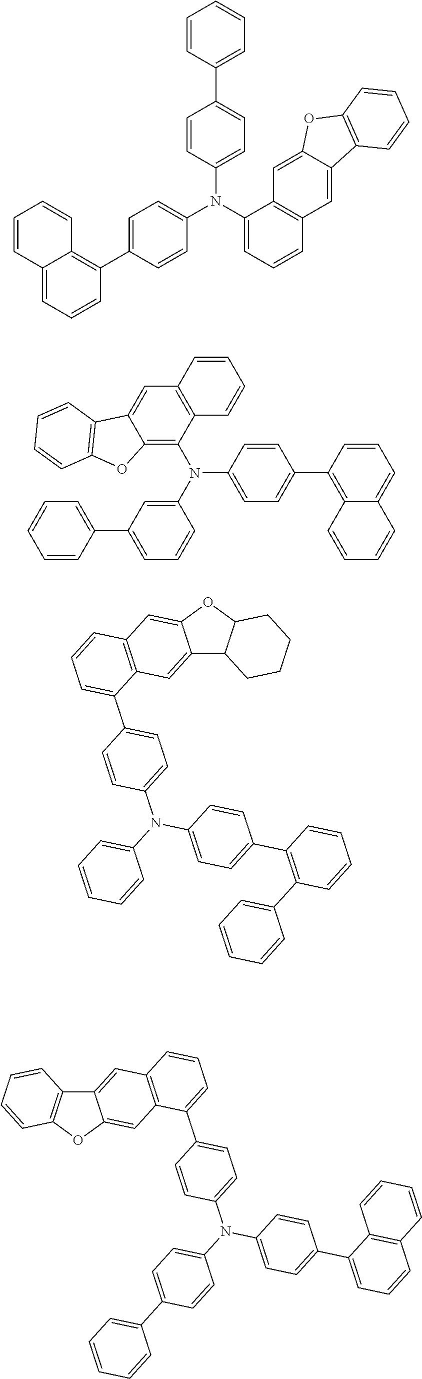 [SMILES: c1ccc(-c2ccc(N(c3ccc(-c4cccc5ccccc45)cc3)c3cccc4cc5c(cc34)oc3ccccc35)cc2)cc1 ; c1ccc(-c2cccc(N(c3ccc(-c4cccc5ccccc45)cc3)c3c4ccccc4cc4c3oc3ccccc34)c2)cc1 ; c1ccc(-c2ccccc2-c2ccc(N(c3ccccc3)c3ccc(-c4cccc5cc6c(cc45)C4CCCCC4O6)cc3)cc2)cc1 ; c1ccc(-c2ccc(N(c3ccc(-c4cccc5ccccc45)cc3)c3ccc(-c4cccc5cc6c(cc45)oc4ccccc46)cc3)cc2)cc1]

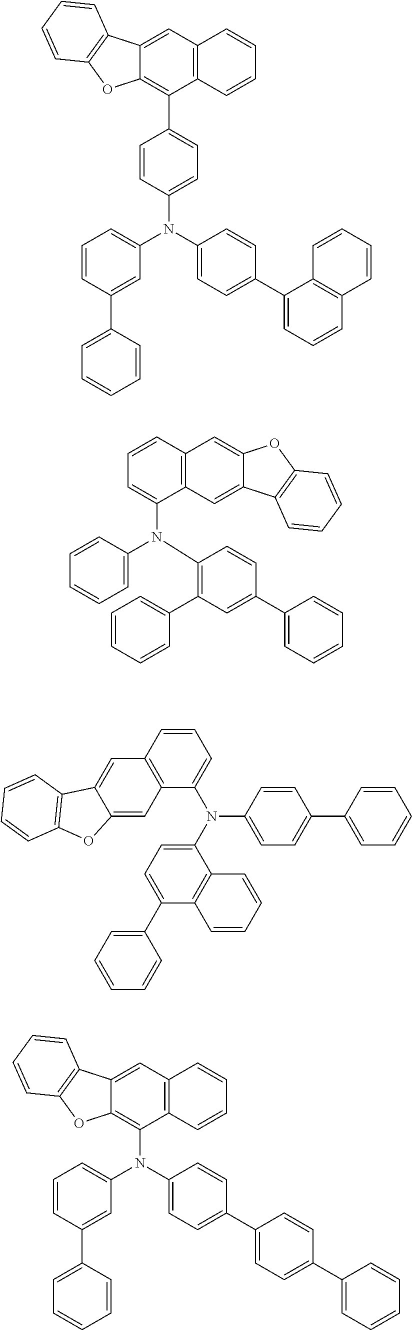 [SMILES: c1ccc(-c2cccc(N(c3ccc(-c4cccc5ccccc45)cc3)c3ccc(-c4c5ccccc5cc5c4oc4ccccc45)cc3)c2)cc1 ; c1ccc(-c2ccc(N(c3ccccc3)c3cccc4cc5oc6ccccc6c5cc34)c(-c3ccccc3)c2)cc1 ; c1ccc(-c2ccc(N(c3cccc4cc5c(cc34)oc3ccccc35)c3ccc(-c4ccccc4)c4ccccc34)cc2)cc1 ; c1ccc(-c2ccc(-c3ccc(N(c4cccc(-c5ccccc5)c4)c4c5ccccc5cc5c4oc4ccccc45)cc3)cc2)cc1]

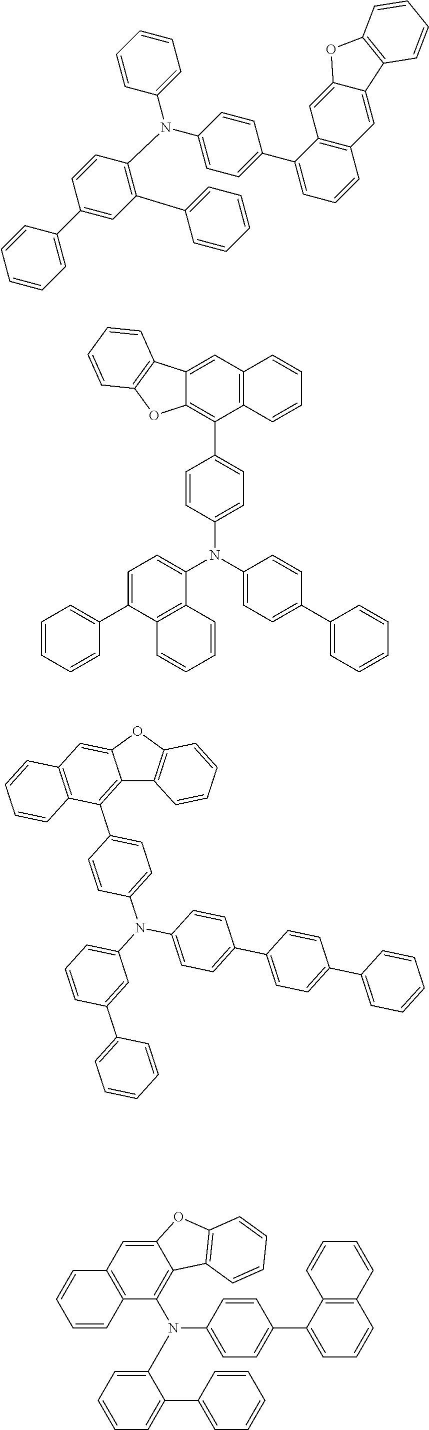 [SMILES: c1ccc(-c2ccc(N(c3ccccc3)c3ccc(-c4cccc5cc6c(cc45)oc4ccccc46)cc3)c(-c3ccccc3)c2)cc1 ; c1ccc(-c2ccc(N(c3ccc(-c4c5ccccc5cc5c4oc4ccccc45)cc3)c3ccc(-c4ccccc4)c4ccccc34)cc2)cc1 ; c1ccc(-c2ccc(-c3ccc(N(c4ccc(-c5c6ccccc6cc6oc7ccccc7c56)cc4)c4cccc(-c5ccccc5)c4)cc3)cc2)cc1 ; c1ccc(-c2ccccc2N(c2ccc(-c3cccc4ccccc34)cc2)c2c3ccccc3cc3oc4ccccc4c23)cc1]

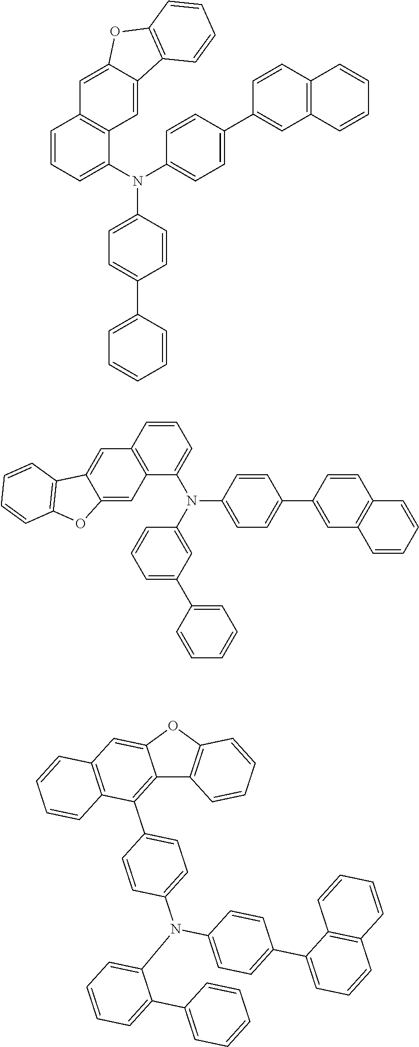

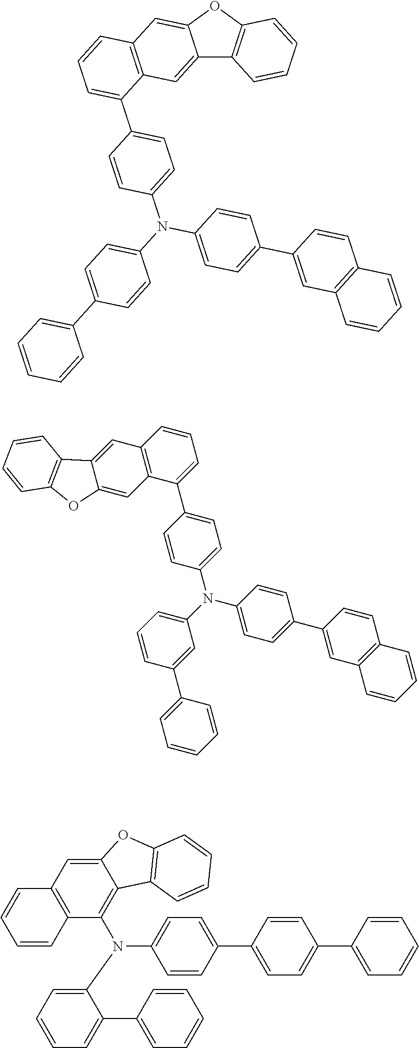 [SMILES: c1ccc(-c2ccc(N(c3ccc(-c4ccc5ccccc5c4)cc3)c3ccc(-c4cccc5cc6oc7ccccc7c6cc45)cc3)cc2)cc1 ; c1ccc(-c2cccc(N(c3ccc(-c4ccc5ccccc5c4)cc3)c3ccc(-c4cccc5cc6c(cc45)oc4ccccc46)cc3)c2)cc1 ; c1ccc(-c2ccc(-c3ccc(N(c4ccccc4-c4ccccc4)c4c5ccccc5cc5oc6ccccc6c45)cc3)cc2)cc1]

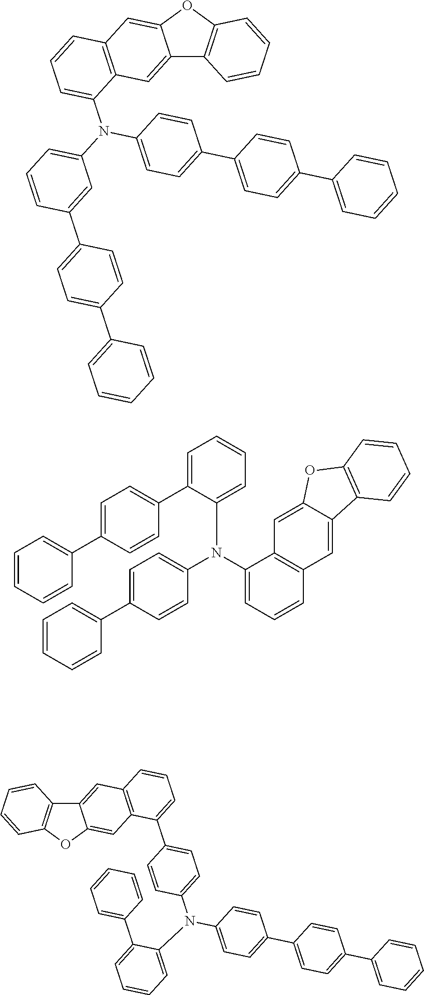 [SMILES: c1ccc(-c2ccc(-c3ccc(N(c4cccc(-c5ccc(-c6ccccc6)cc5)c4)c4cccc5cc6oc7ccccc7c6cc45)cc3)cc2)cc1 ; c1ccc(-c2ccc(-c3ccccc3N(c3ccc(-c4ccccc4)cc3)c3cccc4cc5c(cc34)oc3ccccc35)cc2)cc1 ; c1ccc(-c2ccc(-c3ccc(N(c4ccc(-c5cccc6cc7c(cc56)oc5ccccc57)cc4)c4ccccc4-c4ccccc4)cc3)cc2)cc1]

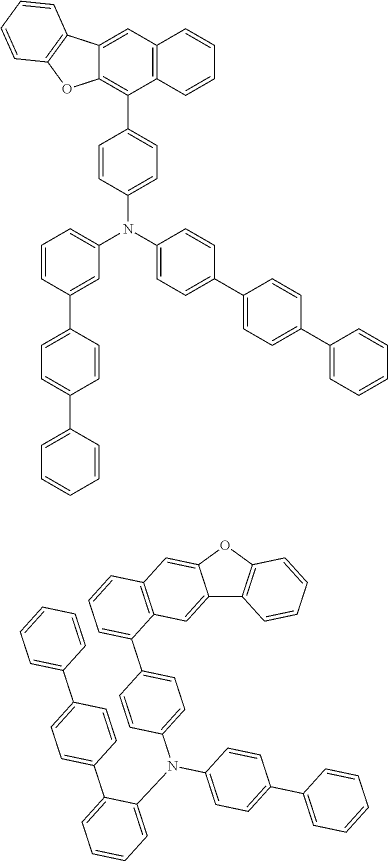 [SMILES: c1ccc(-c2ccc(-c3ccc(N(c4ccc(-c5c6ccccc6cc6c5oc5ccccc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5cc6oc7ccccc7c6cc45)cc3)cc2)cc1]

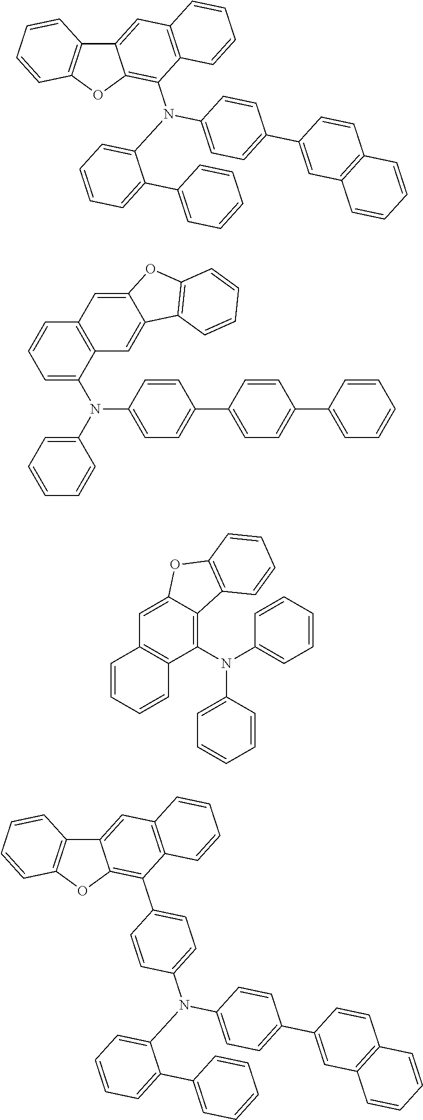

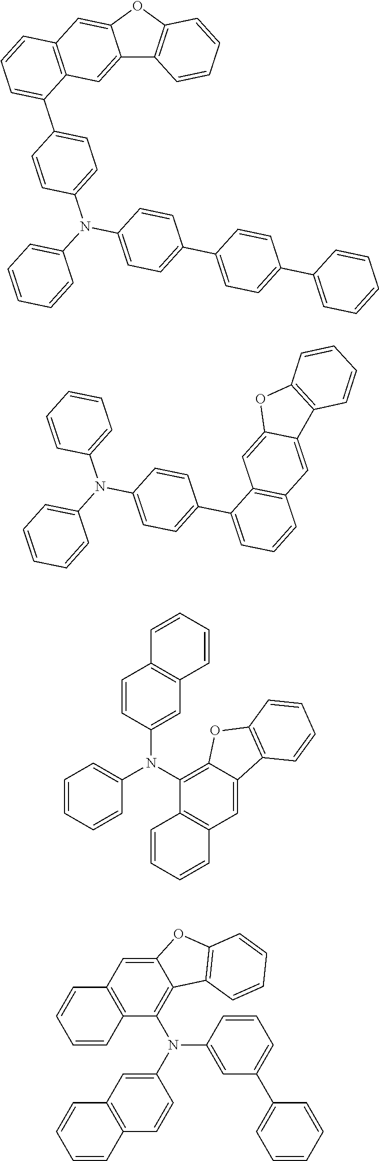

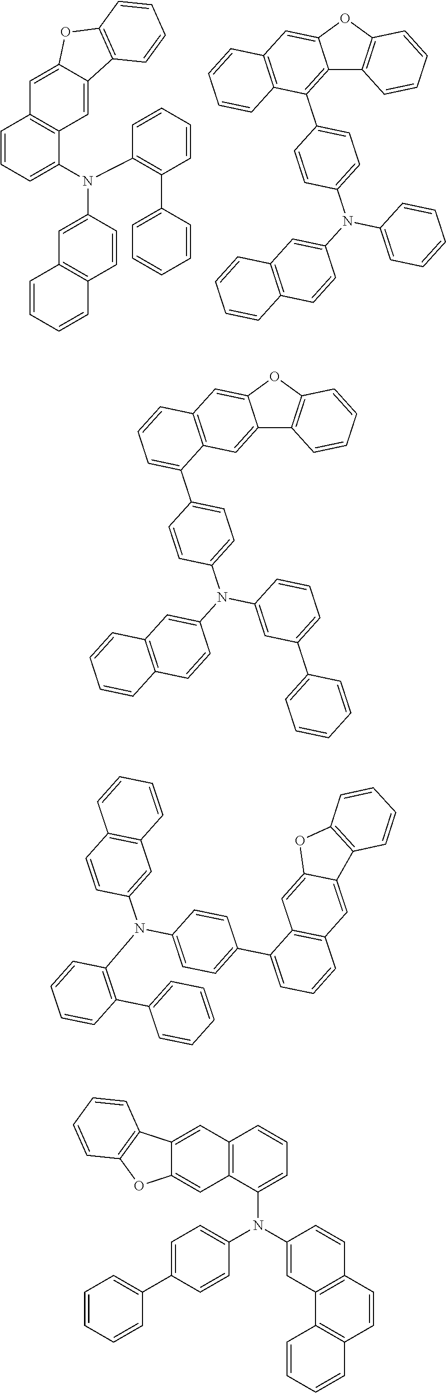

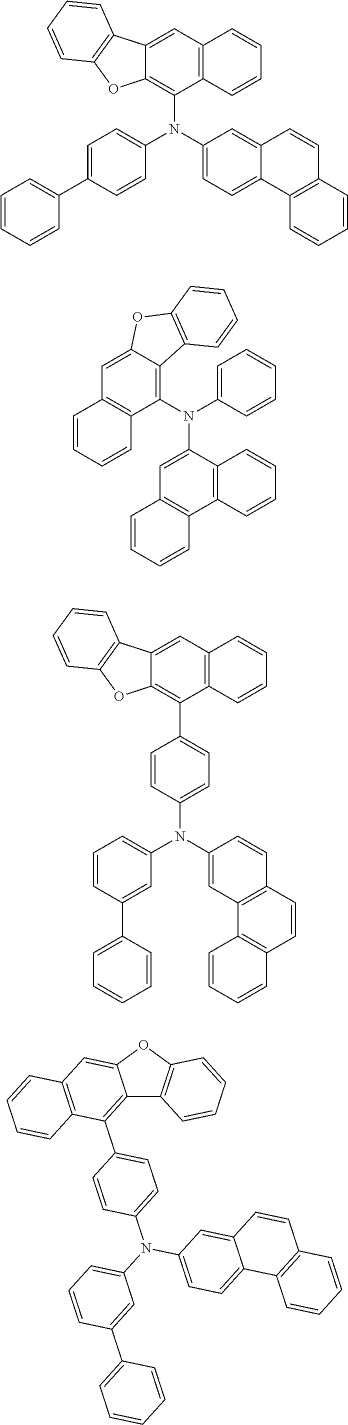 [SMILES: c1ccc(-c2ccc(N(c3ccc4c(ccc5ccccc54)c3)c3c4ccccc4cc4c3oc3ccccc34)cc2)cc1 ; c1ccc(N(c2cc3ccccc3c3ccccc23)c2c3ccccc3cc3oc4ccccc4c23)cc1 ; c1ccc(-c2cccc(N(c3ccc(-c4c5ccccc5cc5c4oc4ccccc45)cc3)c3ccc4ccc5ccccc5c4c3)c2)cc1 ; c1ccc(-c2cccc(N(c3ccc(-c4c5ccccc5cc5oc6ccccc6c45)cc3)c3ccc4c(ccc5ccccc54)c3)c2)cc1]

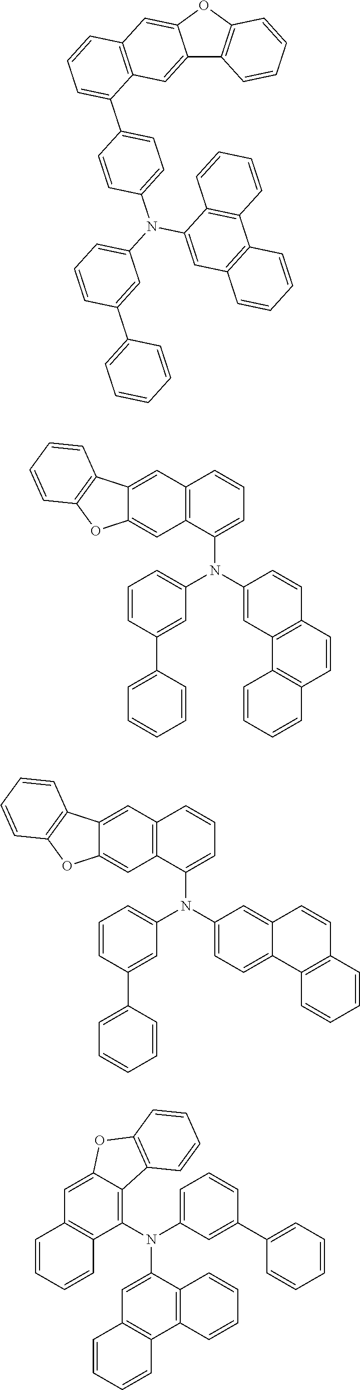

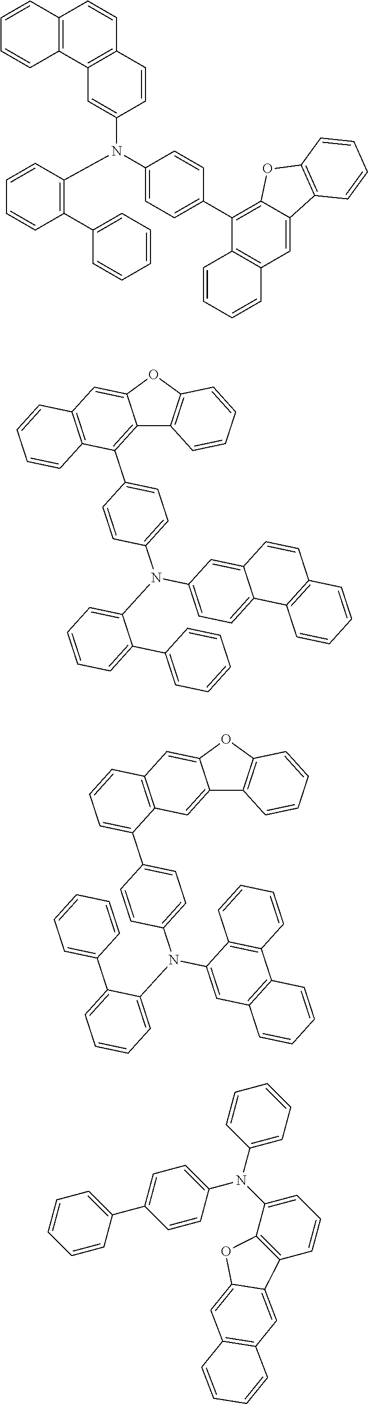 [SMILES: c1ccc(-c2ccccc2N(c2ccc(-c3c4ccccc4cc4c3oc3ccccc34)cc2)c2ccc3ccc4ccccc4c3c2)cc1 ; c1ccc(-c2ccccc2N(c2ccc(-c3c4ccccc4cc4oc5ccccc5c34)cc2)c2ccc3c(ccc4ccccc43)c2)cc1 ; c1ccc(-c2ccccc2N(c2ccc(-c3cccc4cc5oc6ccccc6c5cc34)cc2)c2cc3ccccc3c3ccccc23)cc1 ; c1ccc(-c2ccc(N(c3ccccc3)c3cccc4c3oc3cc5ccccc5cc34)cc2)cc1]

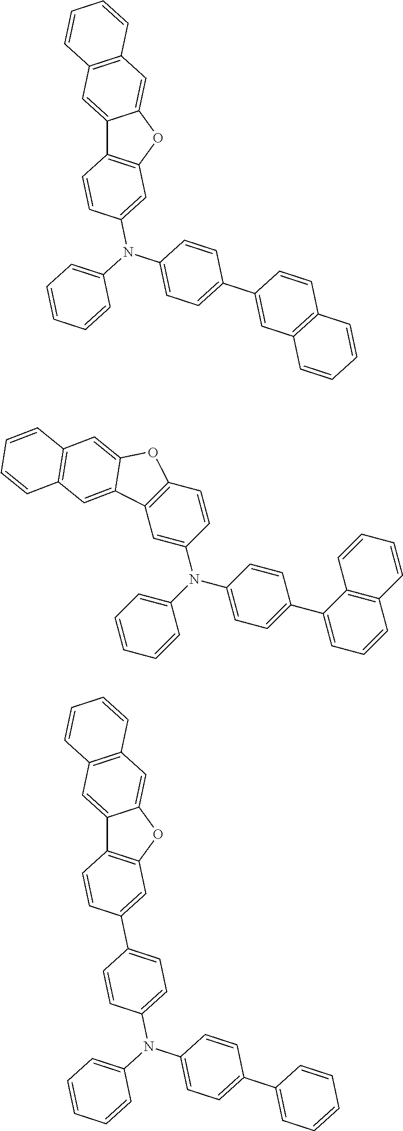 [SMILES: c1ccc(N(c2ccc(-c3ccc4ccccc4c3)cc2)c2ccc3c(c2)oc2cc4ccccc4cc23)cc1 ; c1ccc(N(c2ccc(-c3cccc4ccccc34)cc2)c2ccc3oc4cc5ccccc5cc4c3c2)cc1 ; c1ccc(-c2ccc(N(c3ccccc3)c3ccc(-c4ccc5c(c4)oc4cc6ccccc6cc45)cc3)cc2)cc1]

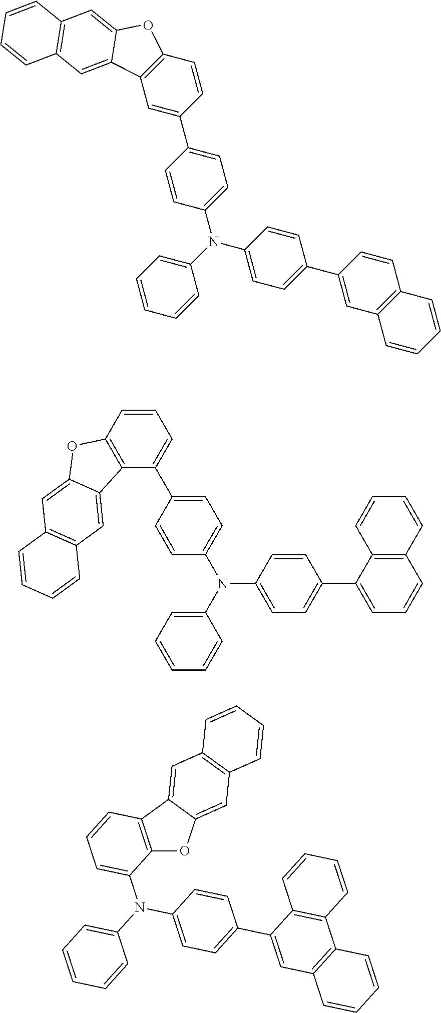 [SMILES: c1ccc(N(c2ccc(-c3ccc4ccccc4c3)cc2)c2ccc(-c3ccc4oc5cc6ccccc6cc5c4c3)cc2)cc1 ; c1ccc(N(c2ccc(-c3cccc4ccccc34)cc2)c2ccc(-c3cccc4oc5cc6ccccc6cc5c34)cc2)cc1 ; c1ccc(N(c2ccc(-c3cc4ccccc4c4ccccc34)cc2)c2cccc3c2oc2cc4ccccc4cc23)cc1]

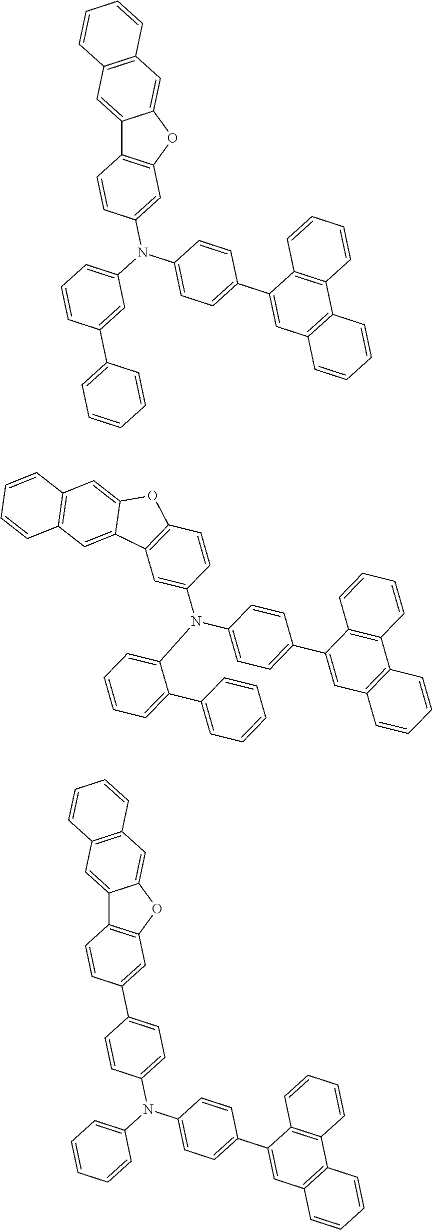 [SMILES: c1ccc(-c2cccc(N(c3ccc(-c4cc5ccccc5c5ccccc45)cc3)c3ccc4c(c3)oc3cc5ccccc5cc34)c2)cc1 ; c1ccc(-c2ccccc2N(c2ccc(-c3cc4ccccc4c4ccccc34)cc2)c2ccc3oc4cc5ccccc5cc4c3c2)cc1 ; c1ccc(N(c2ccc(-c3ccc4c(c3)oc3cc5ccccc5cc34)cc2)c2ccc(-c3cc4ccccc4c4ccccc34)cc2)cc1]

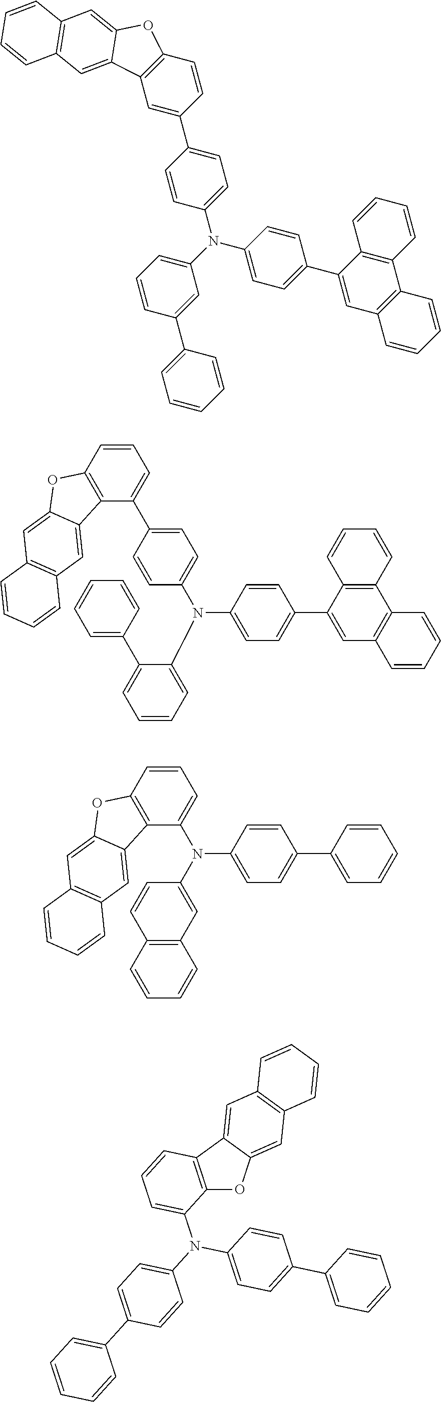 [SMILES: c1ccc(-c2cccc(N(c3ccc(-c4ccc5oc6cc7ccccc7cc6c5c4)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cccc4oc5cc6ccccc6cc5c34)cc2)cc1 ; c1ccc(-c2ccc(N(c3ccc4ccccc4c3)c3cccc4oc5cc6ccccc6cc5c34)cc2)cc1 ; c1ccc(-c2ccc(N(c3ccc(-c4ccccc4)cc3)c3cccc4c3oc3cc5ccccc5cc34)cc2)cc1]

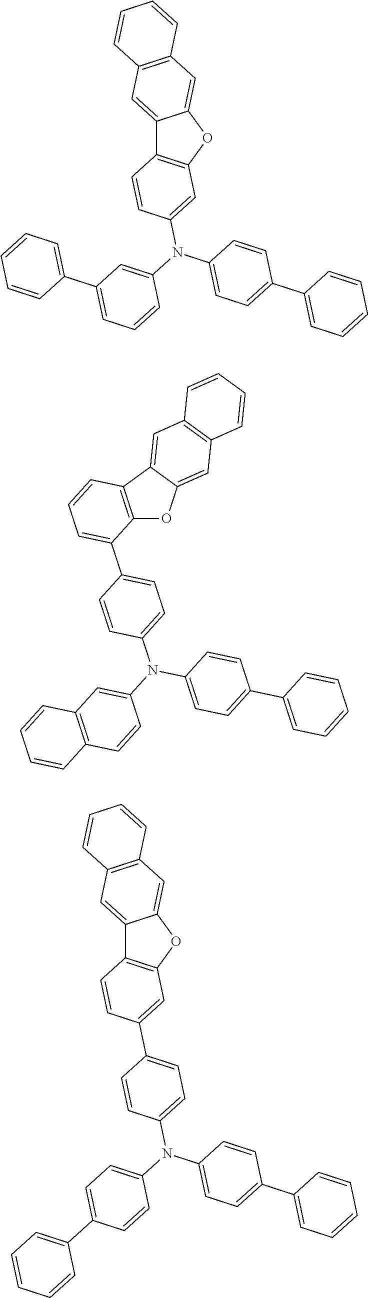 [SMILES: c1ccc(-c2ccc(N(c3cccc(-c4ccccc4)c3)c3ccc4c(c3)oc3cc5ccccc5cc34)cc2)cc1 ; c1ccc(-c2ccc(N(c3ccc(-c4cccc5c4oc4cc6ccccc6cc45)cc3)c3ccc4ccccc4c3)cc2)cc1 ; c1ccc(-c2ccc(N(c3ccc(-c4ccccc4)cc3)c3ccc(-c4ccc5c(c4)oc4cc6ccccc6cc45)cc3)cc2)cc1]

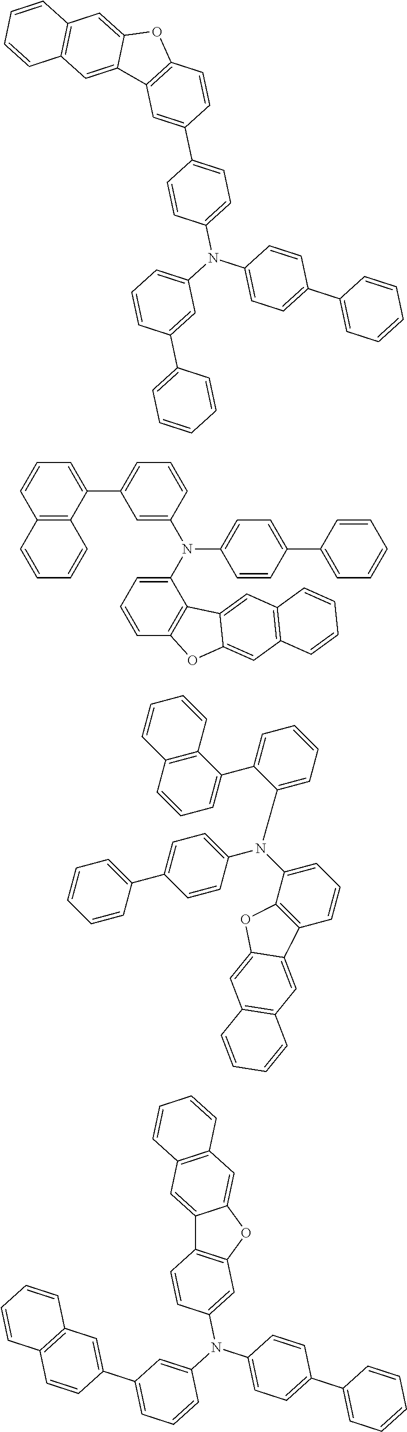

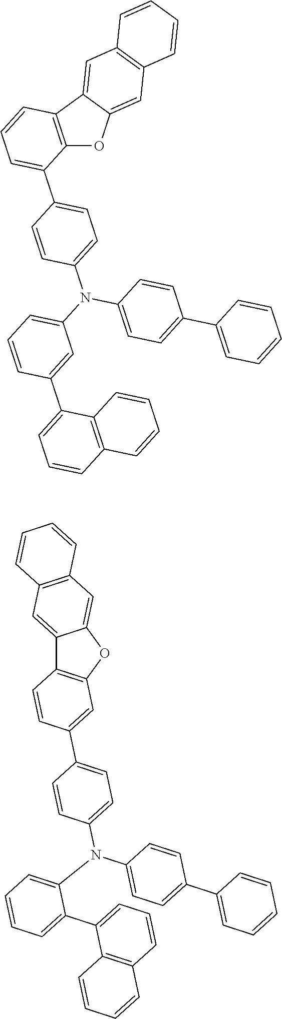 [SMILES: c1ccc(-c2ccc(N(c3ccc(-c4cccc5c4oc4cc6ccccc6cc45)cc3)c3cccc(-c4cccc5ccccc45)c3)cc2)cc1 ; c1ccc(-c2ccc(N(c3ccc(-c4ccc5c(c4)oc4cc6ccccc6cc45)cc3)c3ccccc3-c3cccc4ccccc34)cc2)cc1]

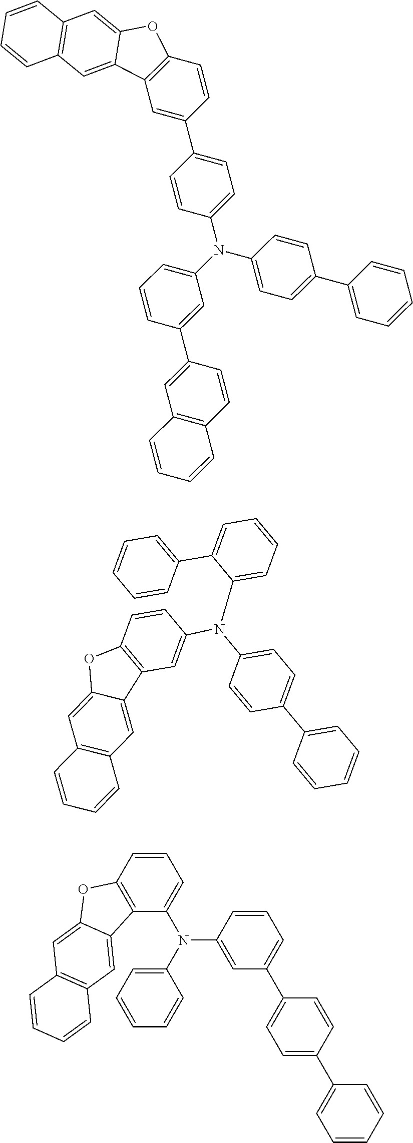 [SMILES: c1ccc(-c2ccc(N(c3ccc(-c4ccc5oc6cc7ccccc7cc6c5c4)cc3)c3cccc(-c4ccc5ccccc5c4)c3)cc2)cc1 ; c1ccc(-c2ccc(N(c3ccc4oc5cc6ccccc6cc5c4c3)c3ccccc3-c3ccccc3)cc2)cc1 ; c1ccc(-c2ccc(-c3cccc(N(c4ccccc4)c4cccc5oc6cc7ccccc7cc6c45)c3)cc2)cc1]

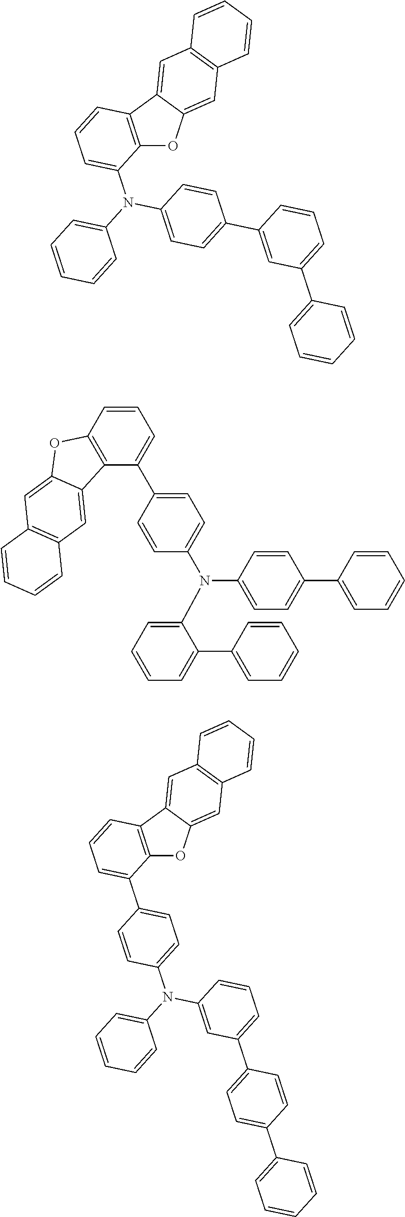

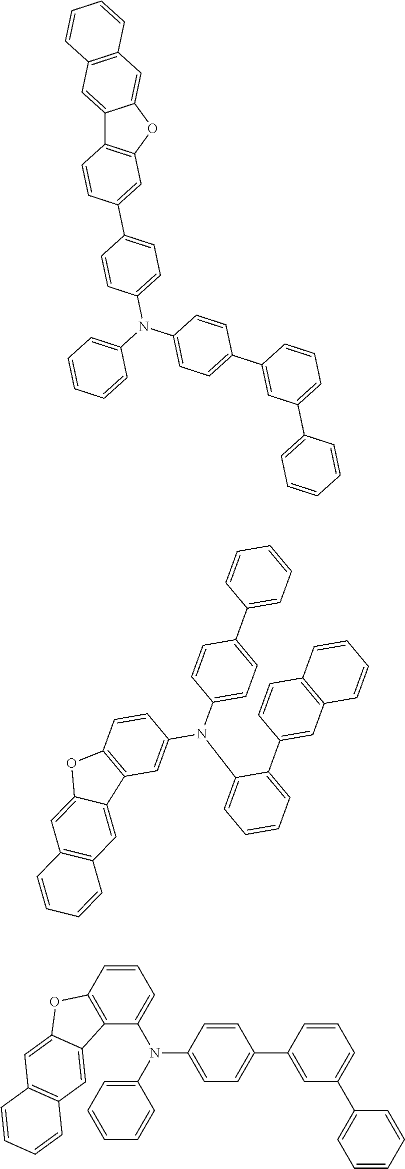 [SMILES: c1ccc(-c2cccc(-c3ccc(N(c4ccccc4)c4ccc(-c5ccc6c(c5)oc5cc7ccccc7cc56)cc4)cc3)c2)cc1 ; c1ccc(-c2ccc(N(c3ccc4oc5cc6ccccc6cc5c4c3)c3ccccc3-c3ccc4ccccc4c3)cc2)cc1 ; c1ccc(-c2cccc(-c3ccc(N(c4ccccc4)c4cccc5oc6cc7ccccc7cc6c45)cc3)c2)cc1]

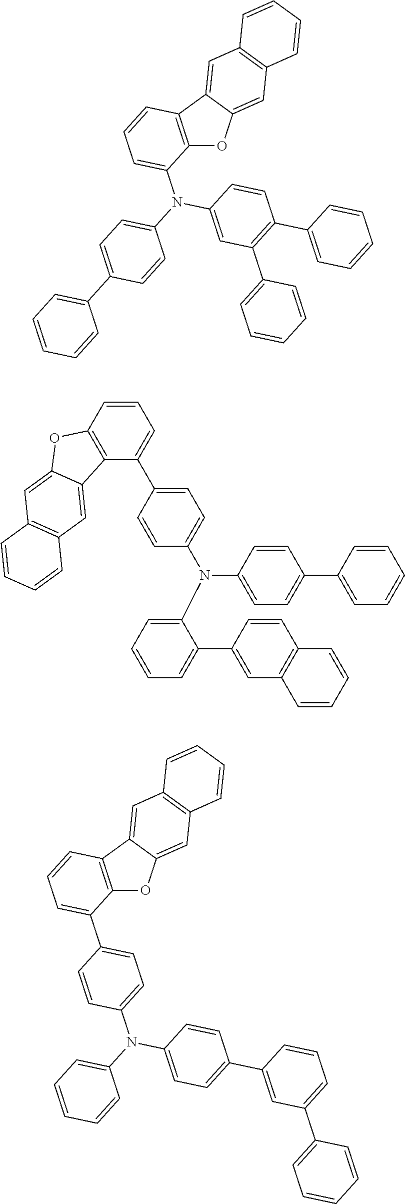 [SMILES: c1ccc(-c2ccc(N(c3ccc(-c4ccccc4)c(-c4ccccc4)c3)c3cccc4c3oc3cc5ccccc5cc34)cc2)cc1 ; c1ccc(-c2ccc(N(c3ccc(-c4cccc5oc6cc7ccccc7cc6c45)cc3)c3ccccc3-c3ccc4ccccc4c3)cc2)cc1 ; c1ccc(-c2cccc(-c3ccc(N(c4ccccc4)c4ccc(-c5cccc6c5oc5cc7ccccc7cc56)cc4)cc3)c2)cc1]

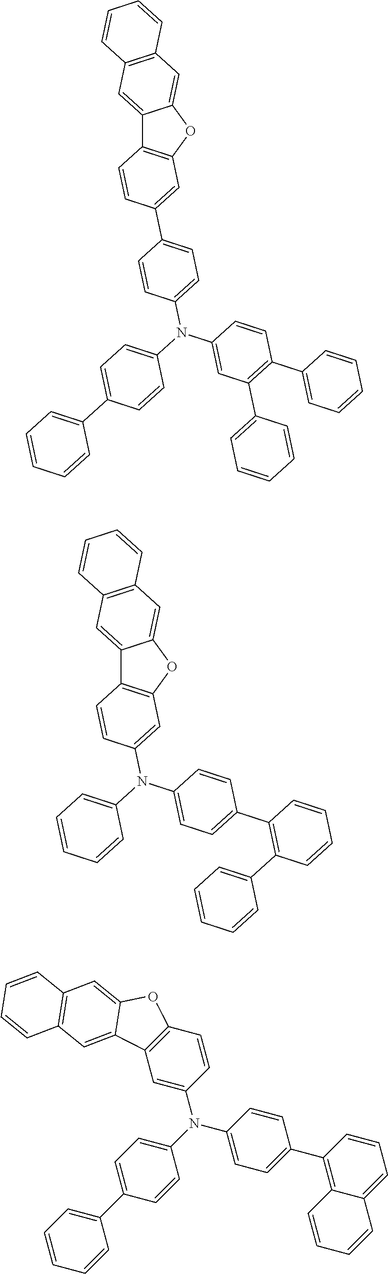 [SMILES: c1ccc(-c2ccc(N(c3ccc(-c4ccc5c(c4)oc4cc6ccccc6cc45)cc3)c3ccc(-c4ccccc4)c(-c4ccccc4)c3)cc2)cc1 ; c1ccc(-c2ccccc2-c2ccc(N(c3ccccc3)c3ccc4c(c3)oc3cc5ccccc5cc34)cc2)cc1 ; c1ccc(-c2ccc(N(c3ccc(-c4cccc5ccccc45)cc3)c3ccc4oc5cc6ccccc6cc5c4c3)cc2)cc1]

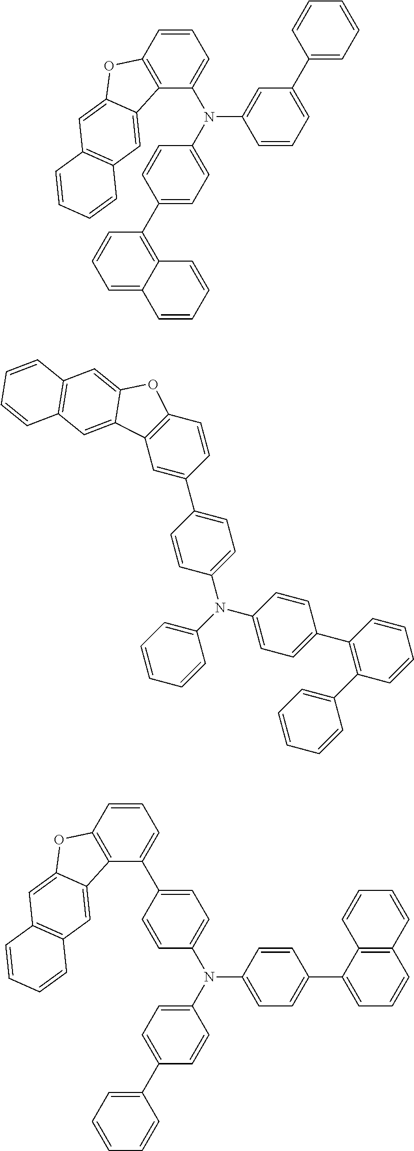 [SMILES: c1ccc(-c2cccc(N(c3ccc(-c4cccc5ccccc45)cc3)c3cccc4oc5cc6ccccc6cc5c34)c2)cc1 ; c1ccc(-c2ccccc2-c2ccc(N(c3ccccc3)c3ccc(-c4ccc5oc6cc7ccccc7cc6c5c4)cc3)cc2)cc1 ; c1ccc(-c2ccc(N(c3ccc(-c4cccc5ccccc45)cc3)c3ccc(-c4cccc5oc6cc7ccccc7cc6c45)cc3)cc2)cc1]

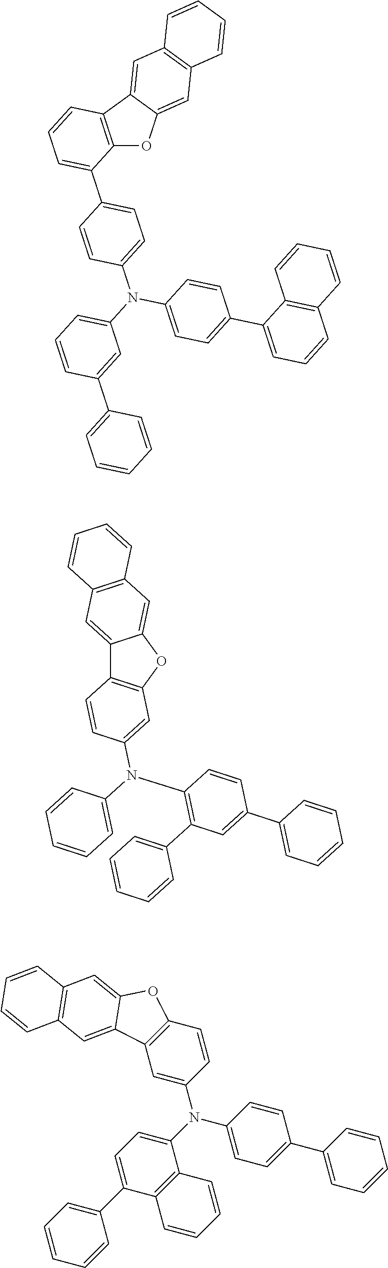 [SMILES: c1ccc(-c2cccc(N(c3ccc(-c4cccc5ccccc45)cc3)c3ccc(-c4cccc5c4oc4cc6ccccc6cc45)cc3)c2)cc1 ; c1ccc(-c2ccc(N(c3ccccc3)c3ccc4c(c3)oc3cc5ccccc5cc34)c(-c3ccccc3)c2)cc1 ; c1ccc(-c2ccc(N(c3ccc4oc5cc6ccccc6cc5c4c3)c3ccc(-c4ccccc4)c4ccccc34)cc2)cc1]

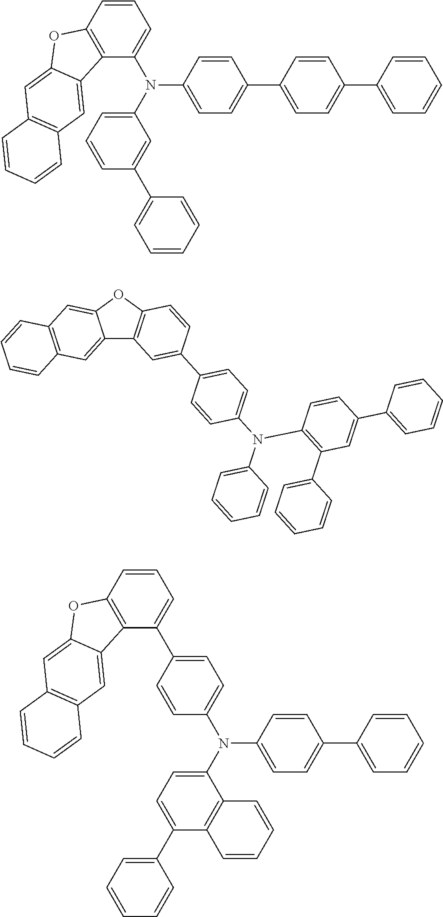 [SMILES: c1ccc(-c2ccc(-c3ccc(N(c4cccc(-c5ccccc5)c4)c4cccc5oc6cc7ccccc7cc6c45)cc3)cc2)cc1 ; c1ccc(-c2ccc(N(c3ccccc3)c3ccc(-c4ccc5oc6cc7ccccc7cc6c5c4)cc3)c(-c3ccccc3)c2)cc1 ; c1ccc(-c2ccc(N(c3ccc(-c4cccc5oc6cc7ccccc7cc6c45)cc3)c3ccc(-c4ccccc4)c4ccccc34)cc2)cc1]

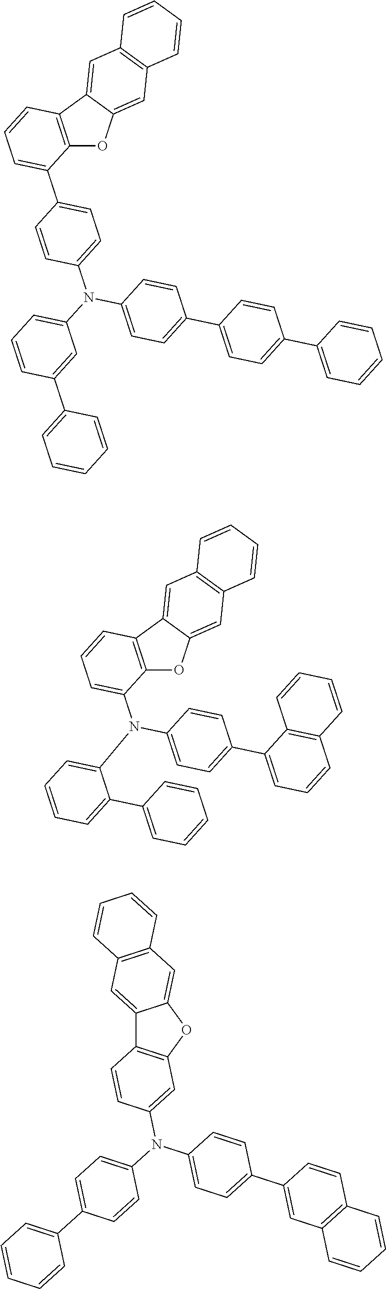 [SMILES: c1ccc(-c2ccc(-c3ccc(N(c4ccc(-c5cccc6c5oc5cc7ccccc7cc56)cc4)c4cccc(-c5ccccc5)c4)cc3)cc2)cc1 ; c1ccc(-c2ccccc2N(c2ccc(-c3cccc4ccccc34)cc2)c2cccc3c2oc2cc4ccccc4cc23)cc1 ; c1ccc(-c2ccc(N(c3ccc(-c4ccc5ccccc5c4)cc3)c3ccc4c(c3)oc3cc5ccccc5cc34)cc2)cc1]

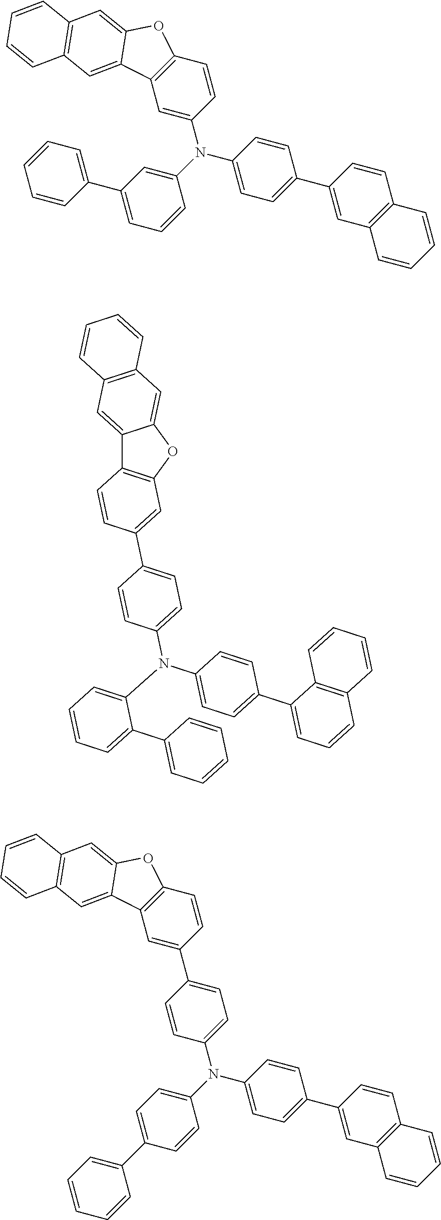 [SMILES: c1ccc(-c2cccc(N(c3ccc(-c4ccc5ccccc5c4)cc3)c3ccc4oc5cc6ccccc6cc5c4c3)c2)cc1 ; c1ccc(-c2ccccc2N(c2ccc(-c3ccc4c(c3)oc3cc5ccccc5cc34)cc2)c2ccc(-c3cccc4ccccc34)cc2)cc1 ; c1ccc(-c2ccc(N(c3ccc(-c4ccc5ccccc5c4)cc3)c3ccc(-c4ccc5oc6cc7ccccc7cc6c5c4)cc3)cc2)cc1]

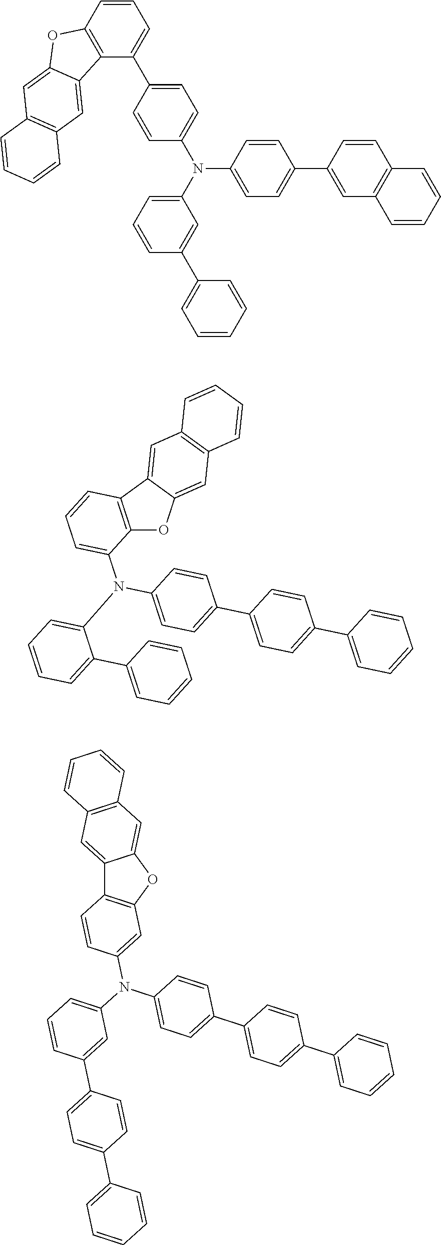 [SMILES: c1ccc(-c2cccc(N(c3ccc(-c4ccc5ccccc5c4)cc3)c3ccc(-c4cccc5oc6cc7ccccc7cc6c45)cc3)c2)cc1 ; c1ccc(-c2ccc(-c3ccc(N(c4ccccc4-c4ccccc4)c4cccc5c4oc4cc6ccccc6cc45)cc3)cc2)cc1 ; c1ccc(-c2ccc(-c3ccc(N(c4cccc(-c5ccc(-c6ccccc6)cc5)c4)c4ccc5c(c4)oc4cc6ccccc6cc45)cc3)cc2)cc1]

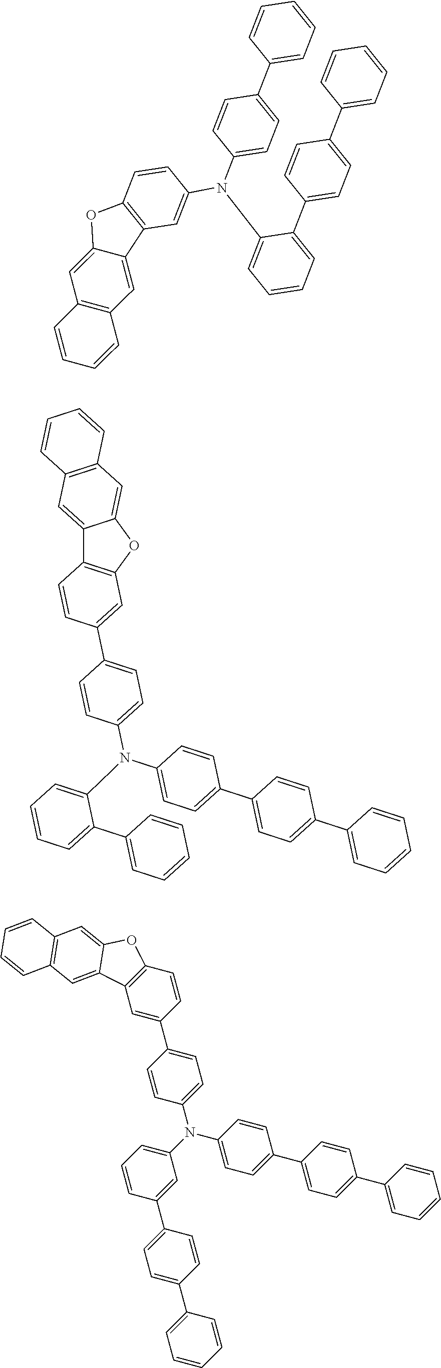

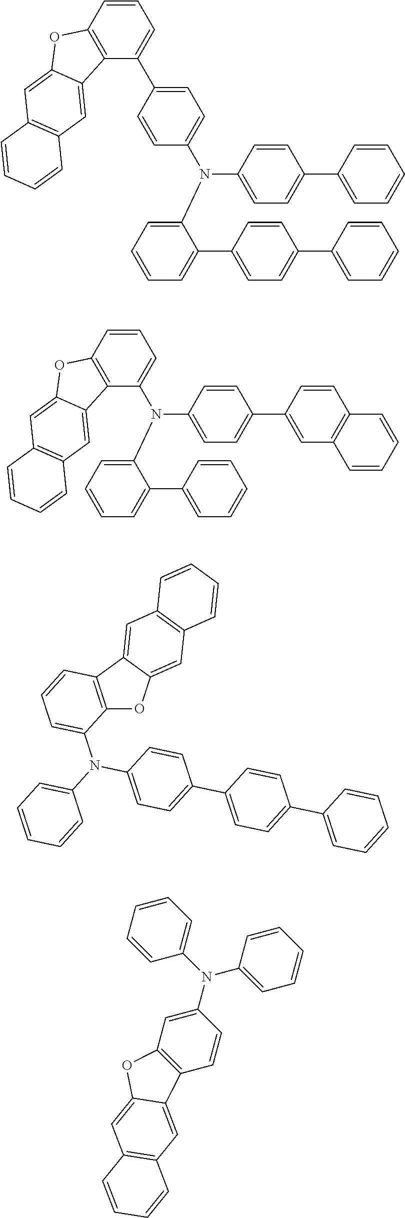 [SMILES: c1ccc(-c2ccc(-c3ccccc3N(c3ccc(-c4ccccc4)cc3)c3ccc(-c4cccc5oc6cc7ccccc7cc6c45)cc3)cc2)cc1 ; c1ccc(-c2ccccc2N(c2ccc(-c3ccc4ccccc4c3)cc2)c2cccc3oc4cc5ccccc5cc4c23)cc1 ; c1ccc(-c2ccc(-c3ccc(N(c4ccccc4)c4cccc5c4oc4cc6ccccc6cc45)cc3)cc2)cc1 ; c1ccc(N(c2ccccc2)c2ccc3c(c2)oc2cc4ccccc4cc23)cc1]

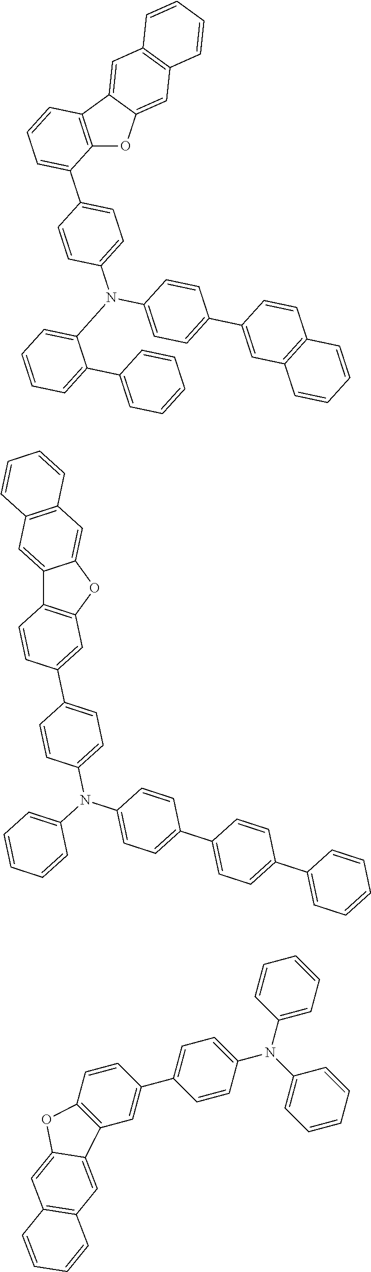 [SMILES: c1ccc(-c2ccccc2N(c2ccc(-c3ccc4ccccc4c3)cc2)c2ccc(-c3cccc4c3oc3cc5ccccc5cc34)cc2)cc1 ; c1ccc(-c2ccc(-c3ccc(N(c4ccccc4)c4ccc(-c5ccc6c(c5)oc5cc7ccccc7cc56)cc4)cc3)cc2)cc1 ; c1ccc(N(c2ccccc2)c2ccc(-c3ccc4oc5cc6ccccc6cc5c4c3)cc2)cc1]

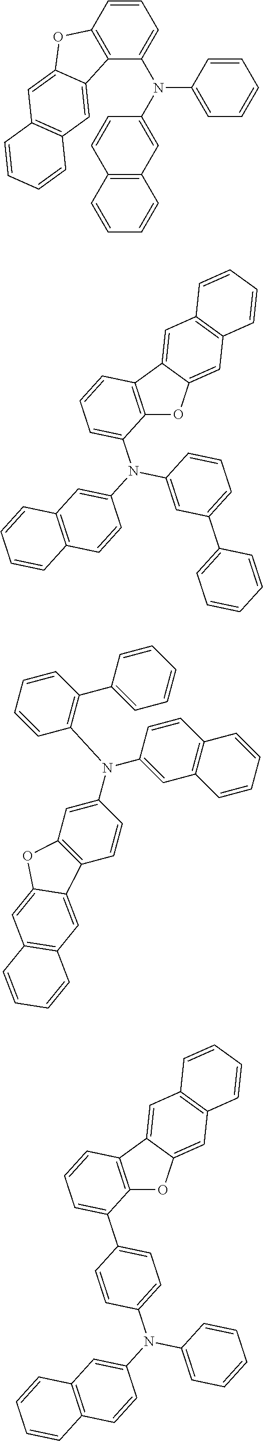 [SMILES: c1ccc(N(c2ccc3ccccc3c2)c2cccc3oc4cc5ccccc5cc4c23)cc1 ; c1ccc(-c2cccc(N(c3ccc4ccccc4c3)c3cccc4c3oc3cc5ccccc5cc34)c2)cc1 ; c1ccc(-c2ccccc2N(c2ccc3ccccc3c2)c2ccc3c(c2)oc2cc4ccccc4cc23)cc1 ; c1ccc(N(c2ccc(-c3cccc4c3oc3cc5ccccc5cc34)cc2)c2ccc3ccccc3c2)cc1]

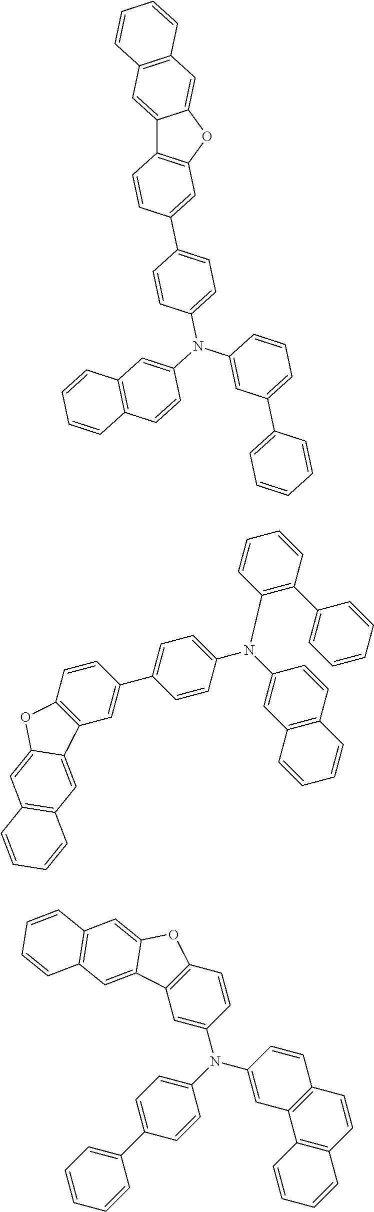

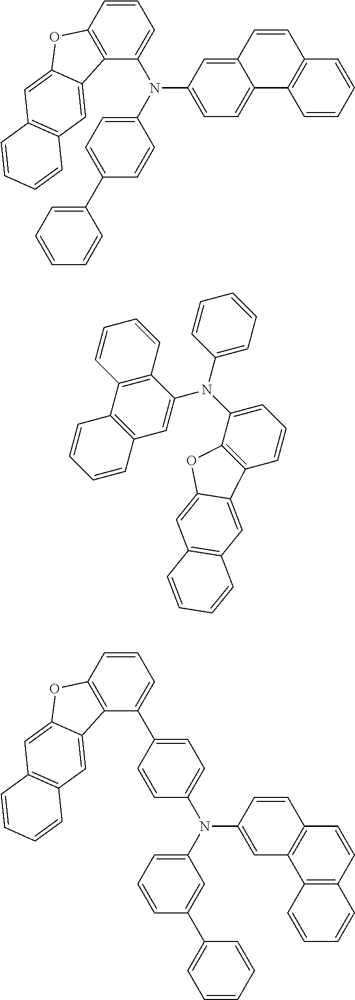 [SMILES: c1ccc(-c2ccc(N(c3ccc4c(ccc5ccccc54)c3)c3cccc4oc5cc6ccccc6cc5c34)cc2)cc1 ; c1ccc(N(c2cc3ccccc3c3ccccc23)c2cccc3c2oc2cc4ccccc4cc23)cc1 ; c1ccc(-c2cccc(N(c3ccc(-c4cccc5oc6cc7ccccc7cc6c45)cc3)c3ccc4ccc5ccccc5c4c3)c2)cc1]

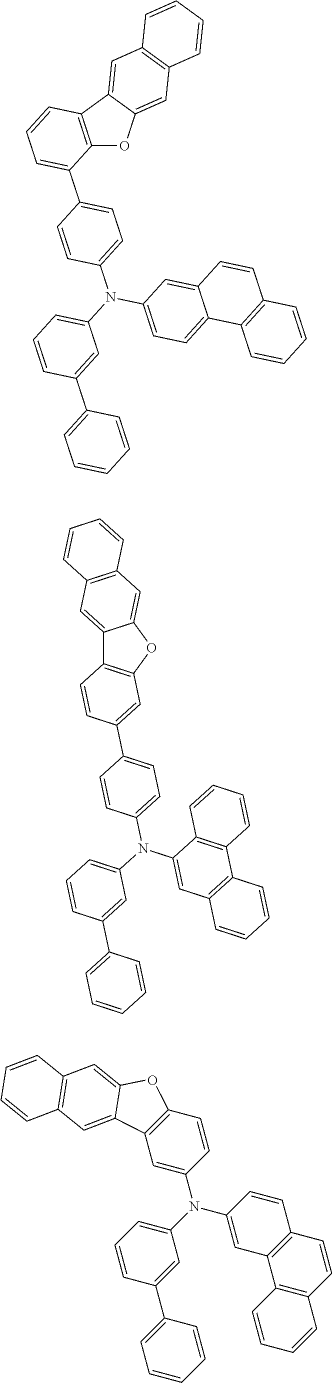

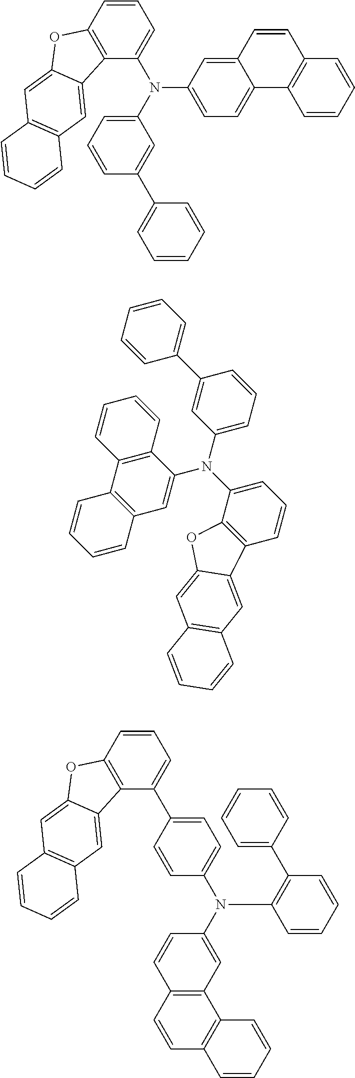

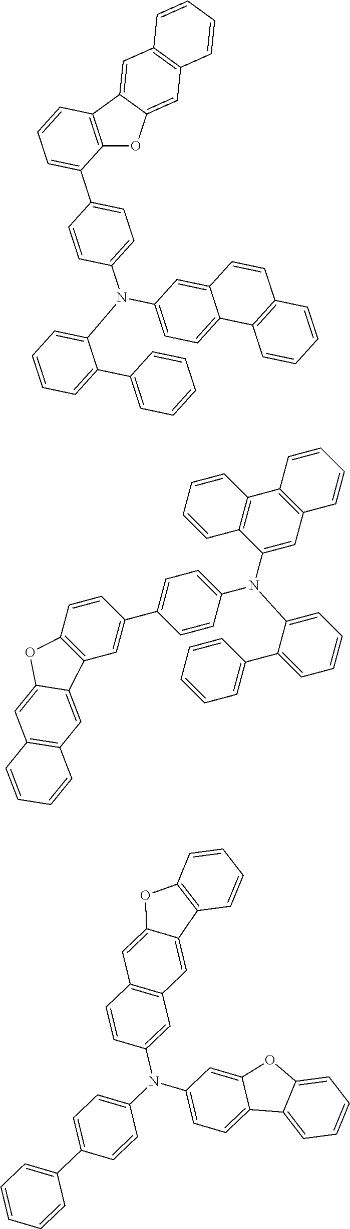 [SMILES: c1ccc(-c2ccccc2N(c2ccc(-c3cccc4c3oc3cc5ccccc5cc34)cc2)c2ccc3c(ccc4ccccc43)c2)cc1 ; c1ccc(-c2ccccc2N(c2ccc(-c3ccc4oc5cc6ccccc6cc5c4c3)cc2)c2cc3ccccc3c3ccccc23)cc1 ; c1ccc(-c2ccc(N(c3ccc4cc5oc6ccccc6c5cc4c3)c3ccc4c(c3)oc3ccccc34)cc2)cc1]

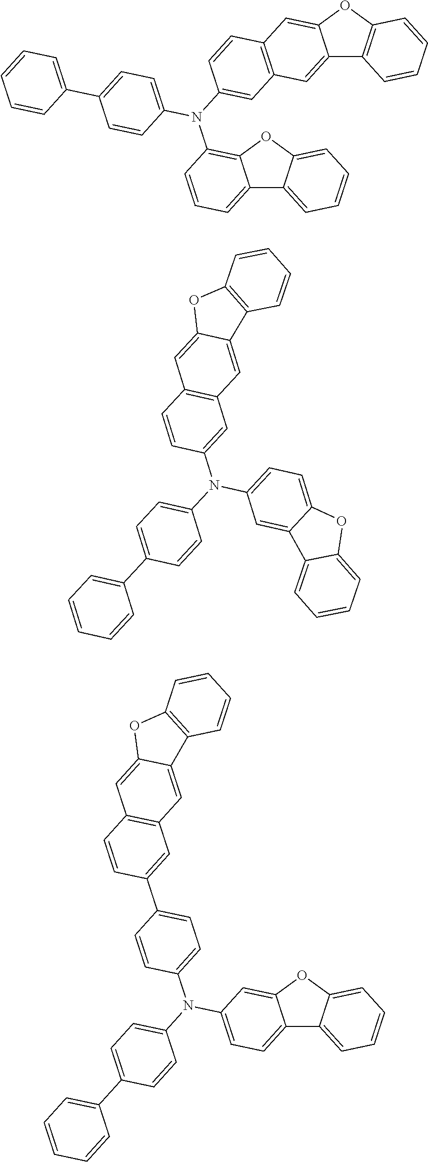

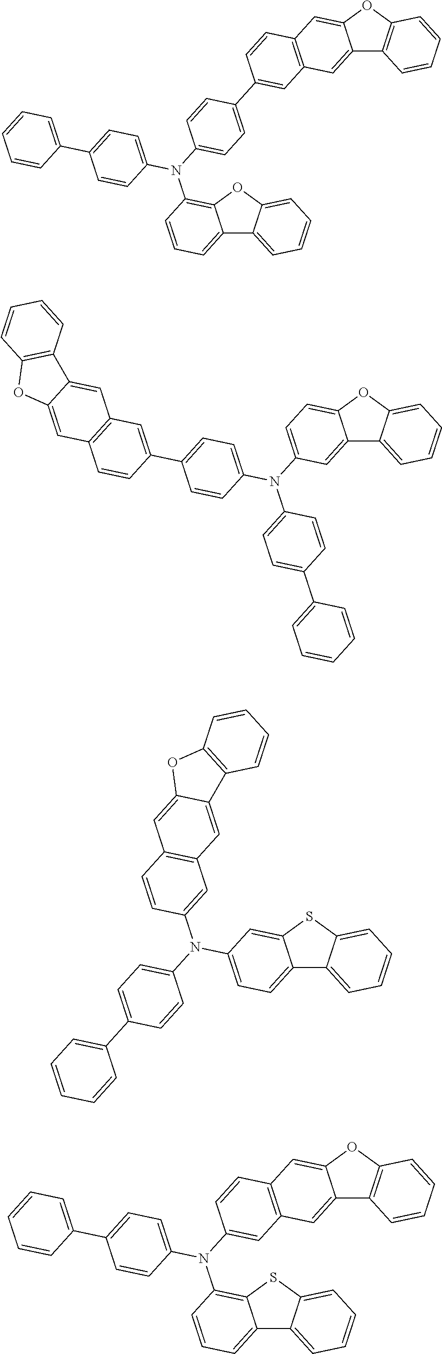 [SMILES: c1ccc(-c2ccc(N(c3ccc(-c4ccc5cc6oc7ccccc7c6cc5c4)cc3)c3cccc4c3oc3ccccc34)cc2)cc1 ; c1ccc(-c2ccc(N(c3ccc(-c4ccc5cc6oc7ccccc7c6cc5c4)cc3)c3ccc4oc5ccccc5c4c3)cc2)cc1 ; c1ccc(-c2ccc(N(c3ccc4cc5oc6ccccc6c5cc4c3)c3ccc4c(c3)sc3ccccc34)cc2)cc1 ; c1ccc(-c2ccc(N(c3ccc4cc5oc6ccccc6c5cc4c3)c3cccc4c3sc3ccccc34)cc2)cc1]

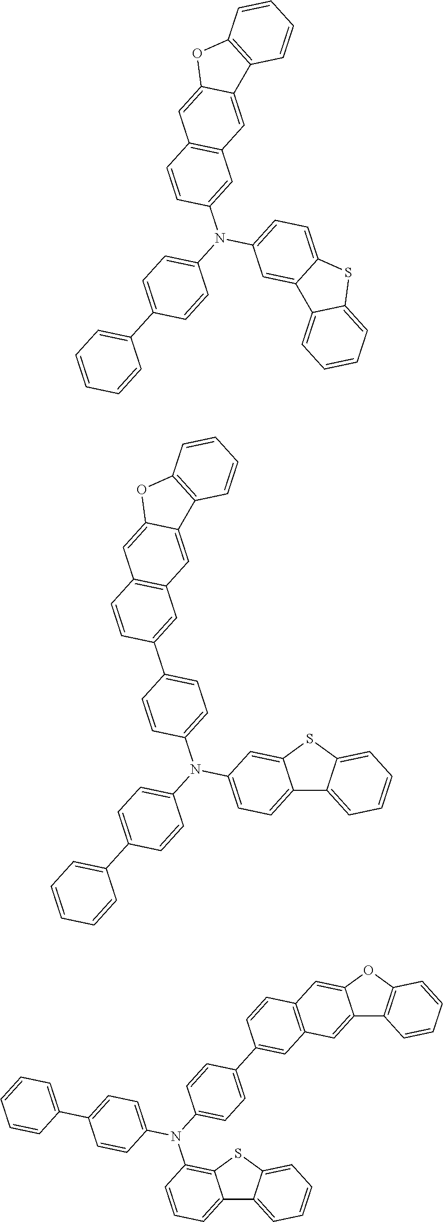 [SMILES: c1ccc(-c2ccc(N(c3ccc4cc5oc6ccccc6c5cc4c3)c3ccc4sc5ccccc5c4c3)cc2)cc1 ; c1ccc(-c2ccc(N(c3ccc(-c4ccc5cc6oc7ccccc7c6cc5c4)cc3)c3ccc4c(c3)sc3ccccc34)cc2)cc1 ; c1ccc(-c2ccc(N(c3ccc(-c4ccc5cc6oc7ccccc7c6cc5c4)cc3)c3cccc4c3sc3ccccc34)cc2)cc1]

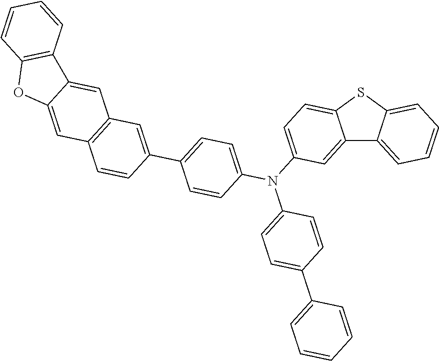 [SMILES: c1ccc(-c2ccc(N(c3ccc(-c4ccc5cc6oc7ccccc7c6cc5c4)cc3)c3ccc4sc5ccccc5c4c3)cc2)cc1]

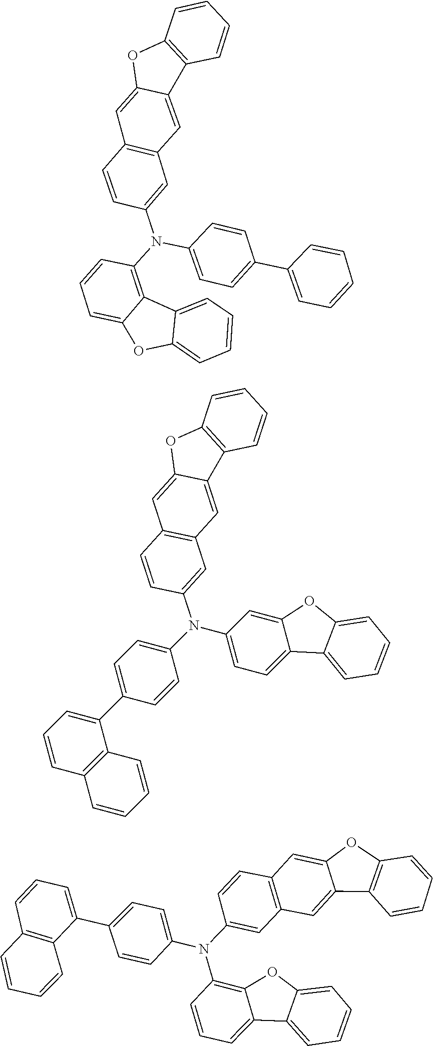

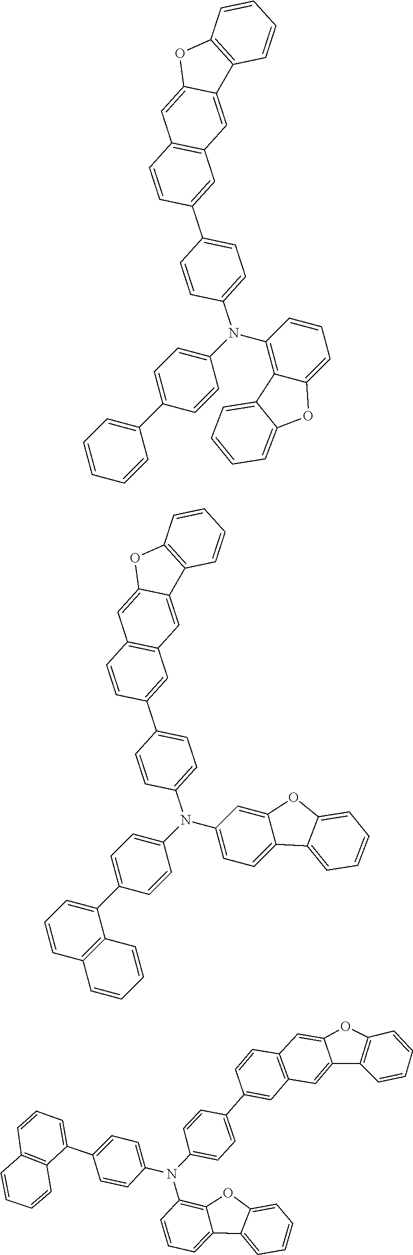 [SMILES: c1ccc(-c2ccc(N(c3ccc(-c4ccc5cc6oc7ccccc7c6cc5c4)cc3)c3cccc4oc5ccccc5c34)cc2)cc1 ; c1ccc2c(-c3ccc(N(c4ccc(-c5ccc6cc7oc8ccccc8c7cc6c5)cc4)c4ccc5c(c4)oc4ccccc45)cc3)cccc2c1 ; c1ccc2c(-c3ccc(N(c4ccc(-c5ccc6cc7oc8ccccc8c7cc6c5)cc4)c4cccc5c4oc4ccccc45)cc3)cccc2c1]

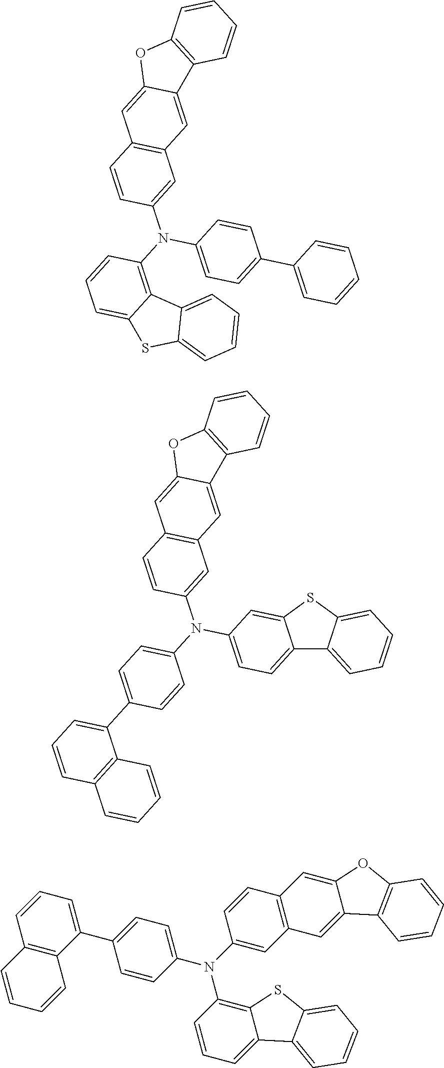

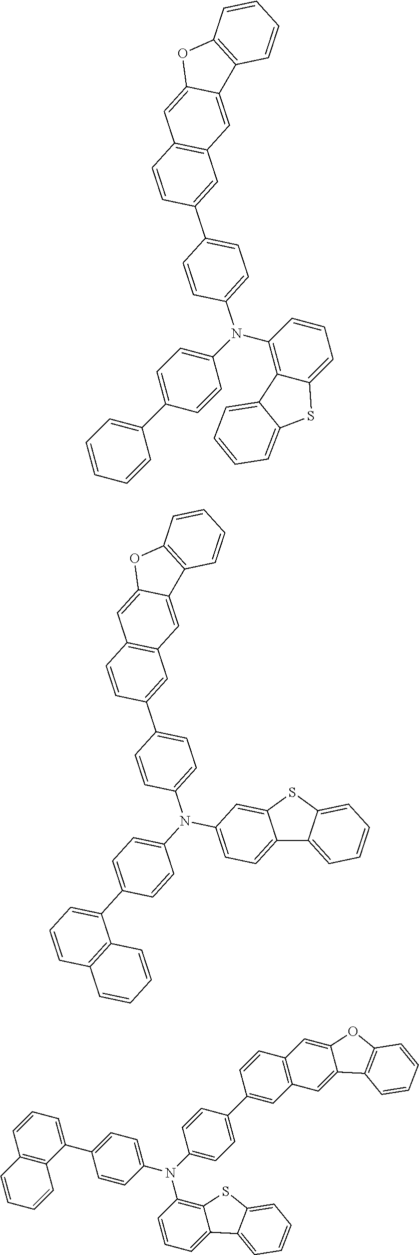 [SMILES: c1ccc(-c2ccc(N(c3ccc(-c4ccc5cc6oc7ccccc7c6cc5c4)cc3)c3cccc4sc5ccccc5c34)cc2)cc1 ; c1ccc2c(-c3ccc(N(c4ccc(-c5ccc6cc7oc8ccccc8c7cc6c5)cc4)c4ccc5c(c4)sc4ccccc45)cc3)cccc2c1 ; c1ccc2c(-c3ccc(N(c4ccc(-c5ccc6cc7oc8ccccc8c7cc6c5)cc4)c4cccc5c4sc4ccccc45)cc3)cccc2c1]

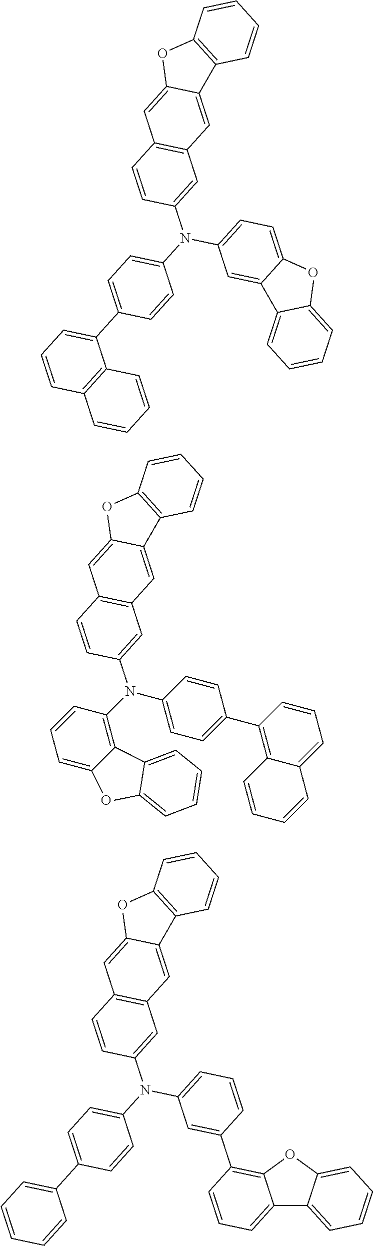

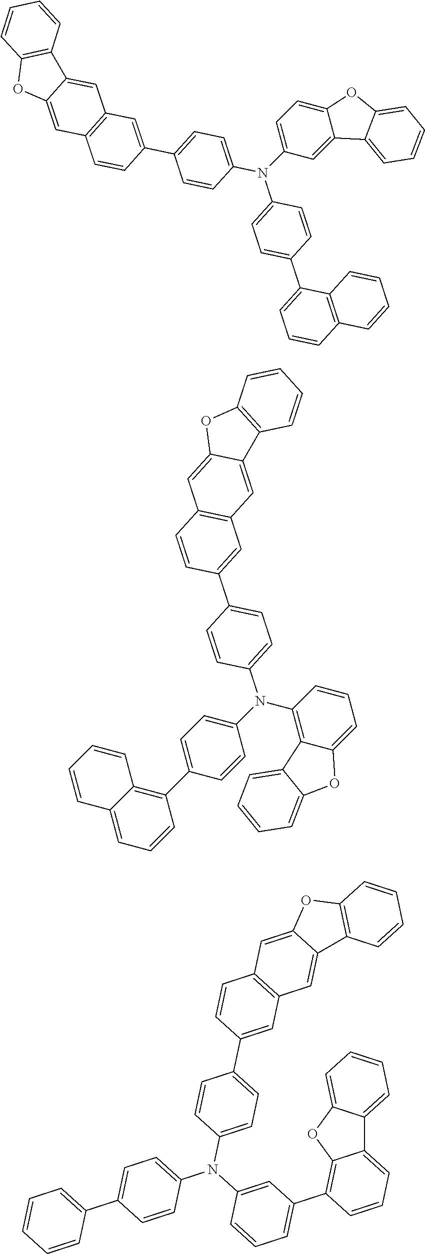

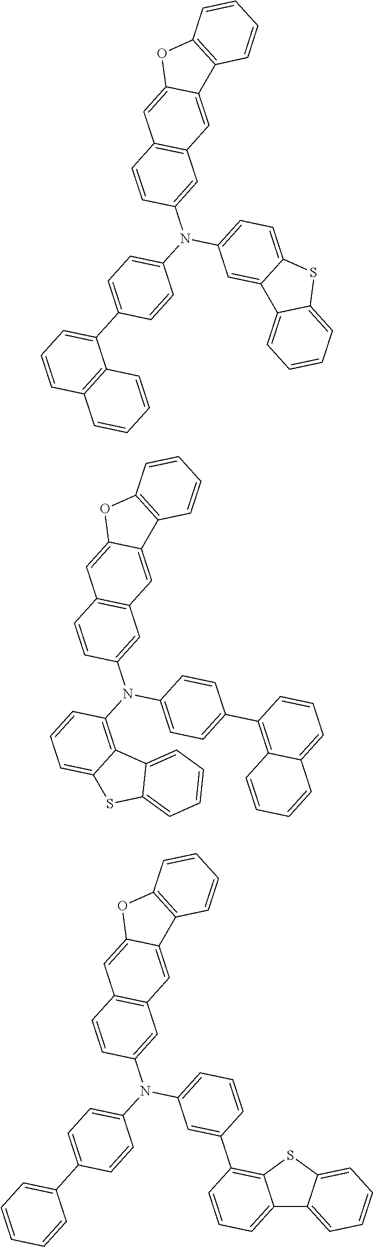

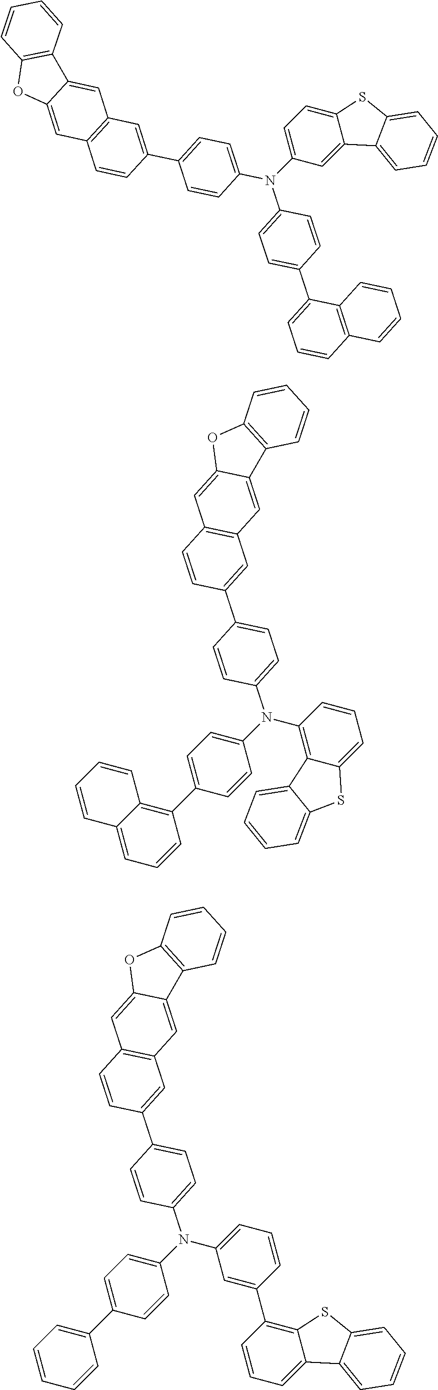

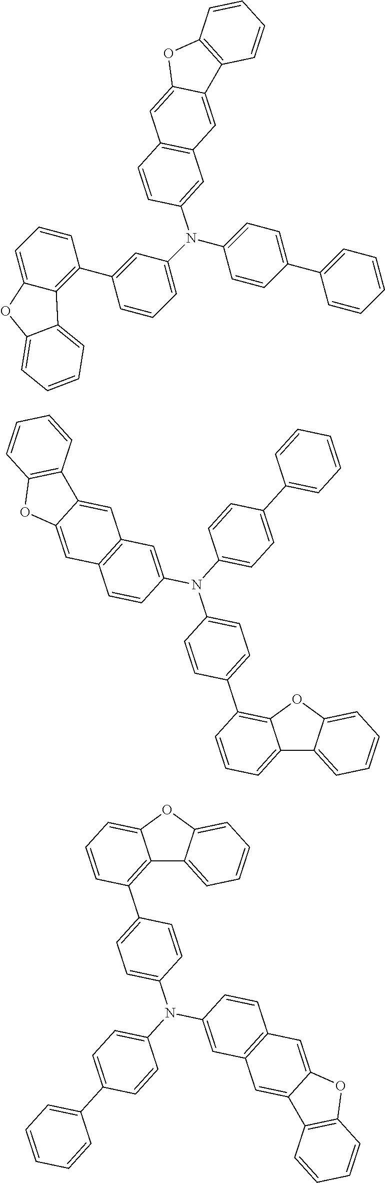

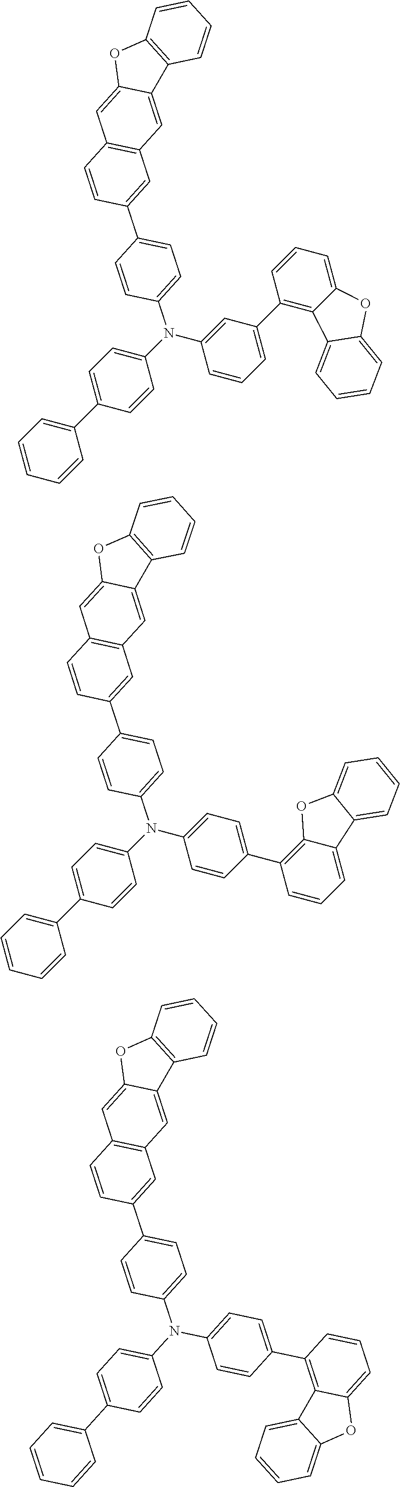 [SMILES: c1ccc(-c2ccc(N(c3ccc(-c4ccc5cc6oc7ccccc7c6cc5c4)cc3)c3cccc(-c4cccc5oc6ccccc6c45)c3)cc2)cc1 ; c1ccc(-c2ccc(N(c3ccc(-c4ccc5cc6oc7ccccc7c6cc5c4)cc3)c3ccc(-c4cccc5c4oc4ccccc45)cc3)cc2)cc1 ; c1ccc(-c2ccc(N(c3ccc(-c4ccc5cc6oc7ccccc7c6cc5c4)cc3)c3ccc(-c4cccc5oc6ccccc6c45)cc3)cc2)cc1]

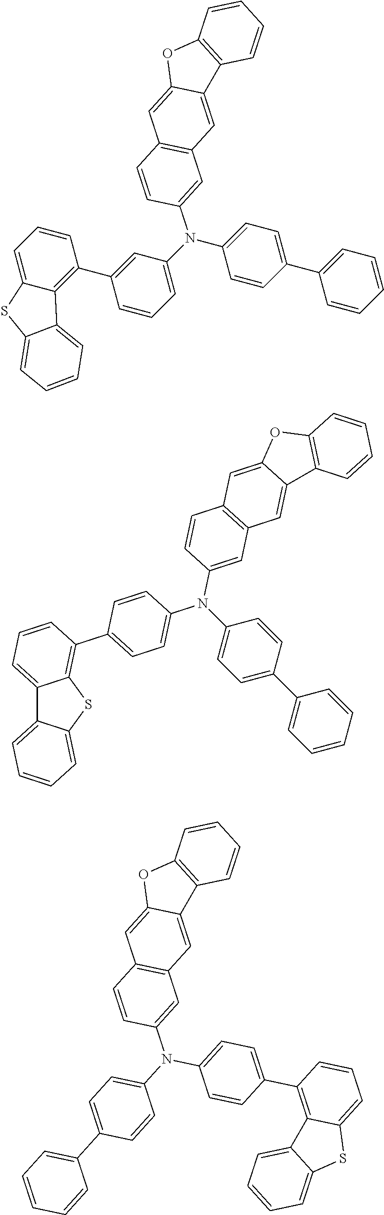 [SMILES: c1ccc(-c2ccc(N(c3cccc(-c4cccc5sc6ccccc6c45)c3)c3ccc4cc5oc6ccccc6c5cc4c3)cc2)cc1 ; c1ccc(-c2ccc(N(c3ccc(-c4cccc5c4sc4ccccc45)cc3)c3ccc4cc5oc6ccccc6c5cc4c3)cc2)cc1 ; c1ccc(-c2ccc(N(c3ccc(-c4cccc5sc6ccccc6c45)cc3)c3ccc4cc5oc6ccccc6c5cc4c3)cc2)cc1]

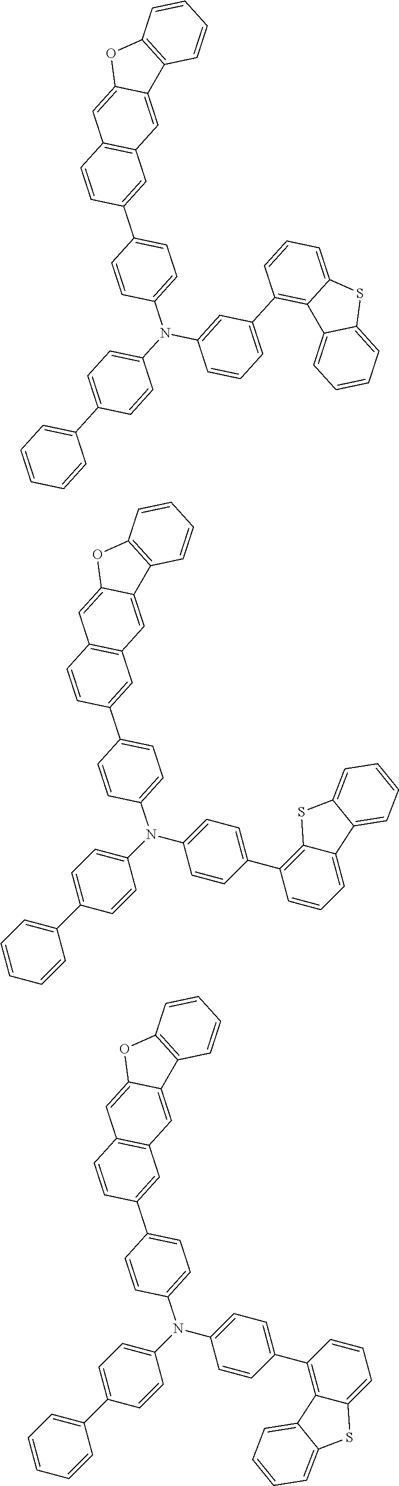 [SMILES: c1ccc(-c2ccc(N(c3ccc(-c4ccc5cc6oc7ccccc7c6cc5c4)cc3)c3cccc(-c4cccc5sc6ccccc6c45)c3)cc2)cc1 ; c1ccc(-c2ccc(N(c3ccc(-c4ccc5cc6oc7ccccc7c6cc5c4)cc3)c3ccc(-c4cccc5c4sc4ccccc45)cc3)cc2)cc1 ; c1ccc(-c2ccc(N(c3ccc(-c4ccc5cc6oc7ccccc7c6cc5c4)cc3)c3ccc(-c4cccc5sc6ccccc6c45)cc3)cc2)cc1]

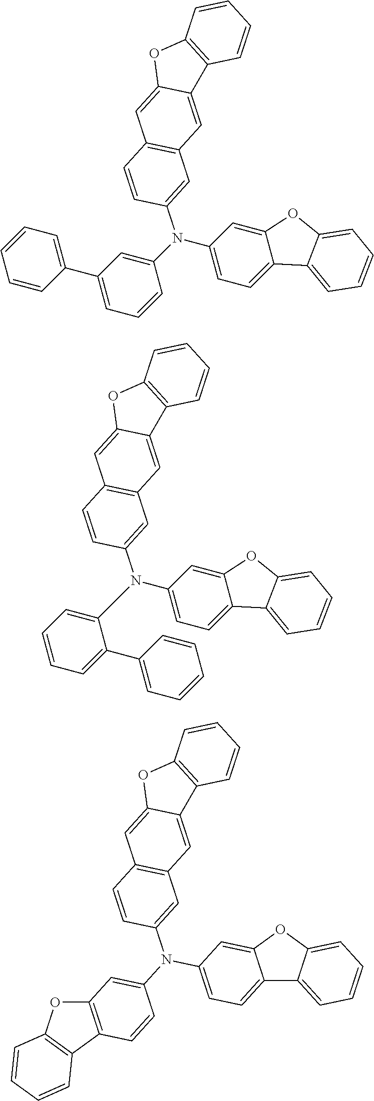

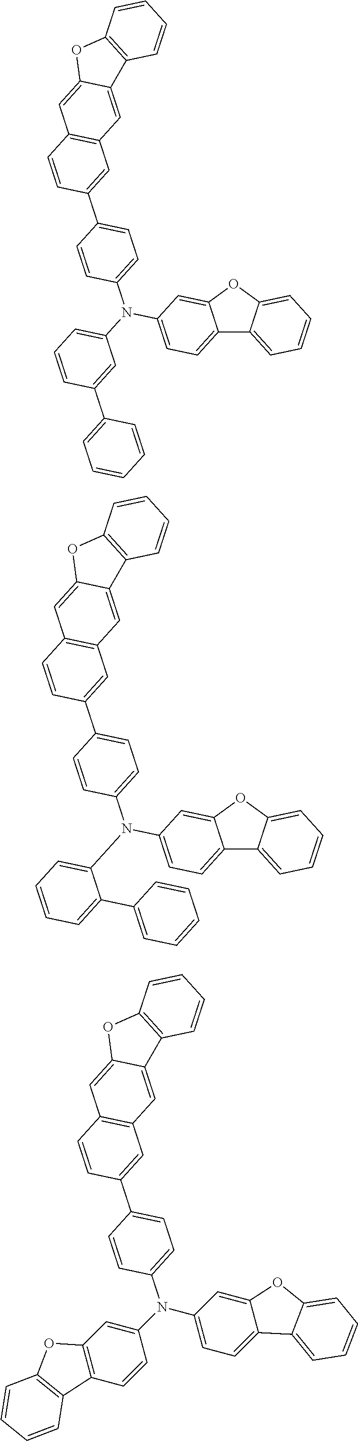

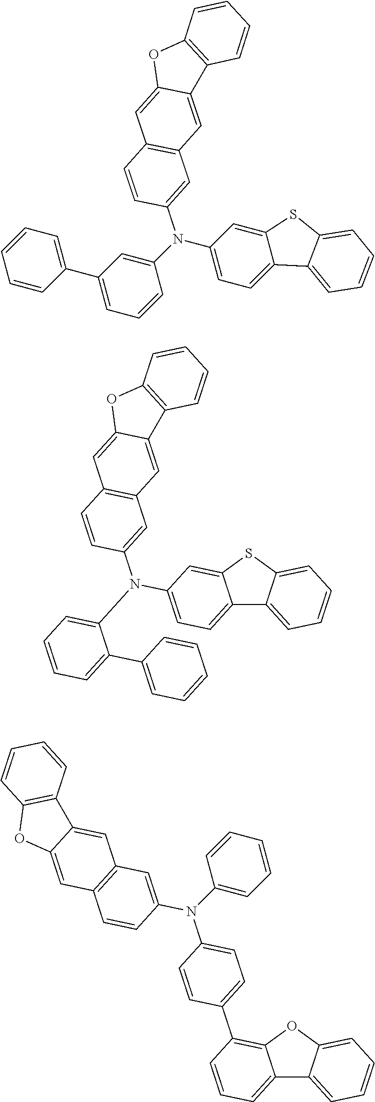

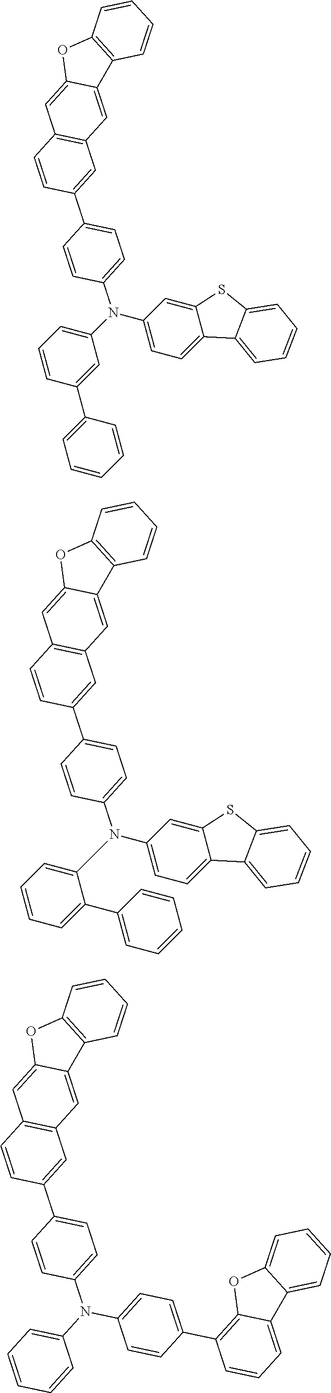

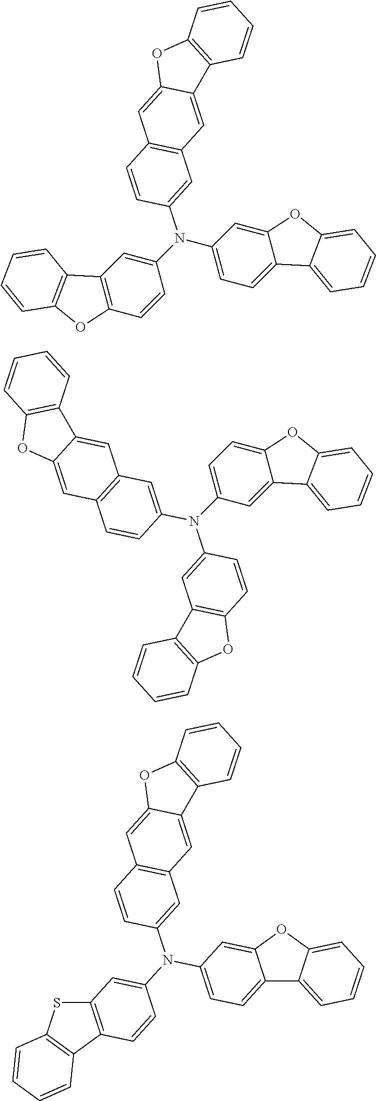

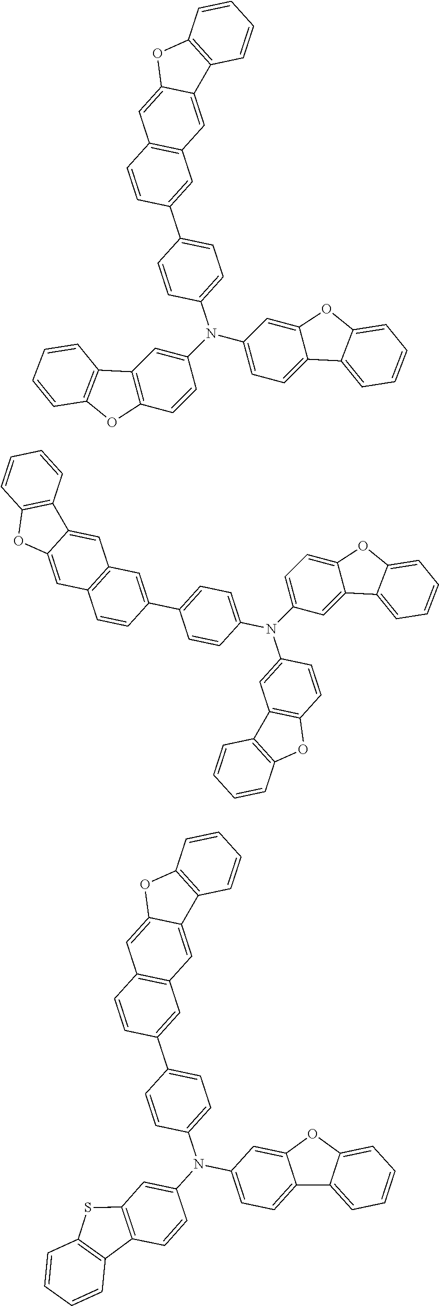

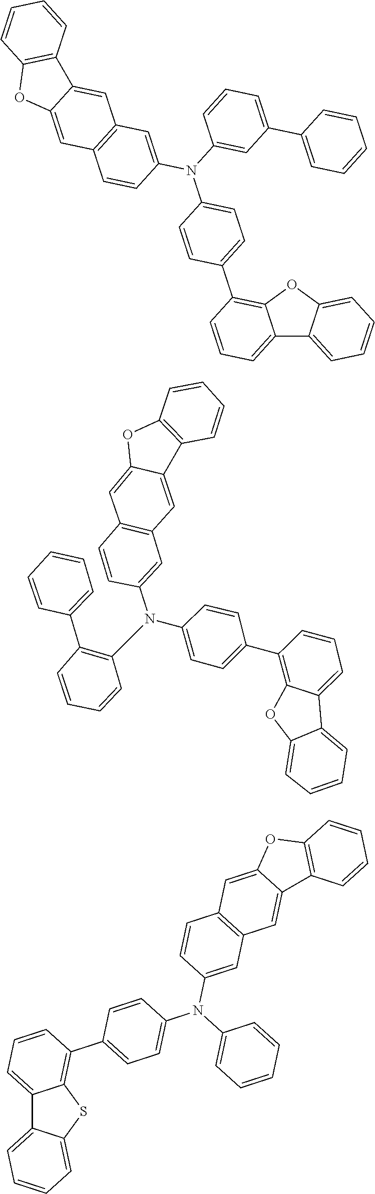

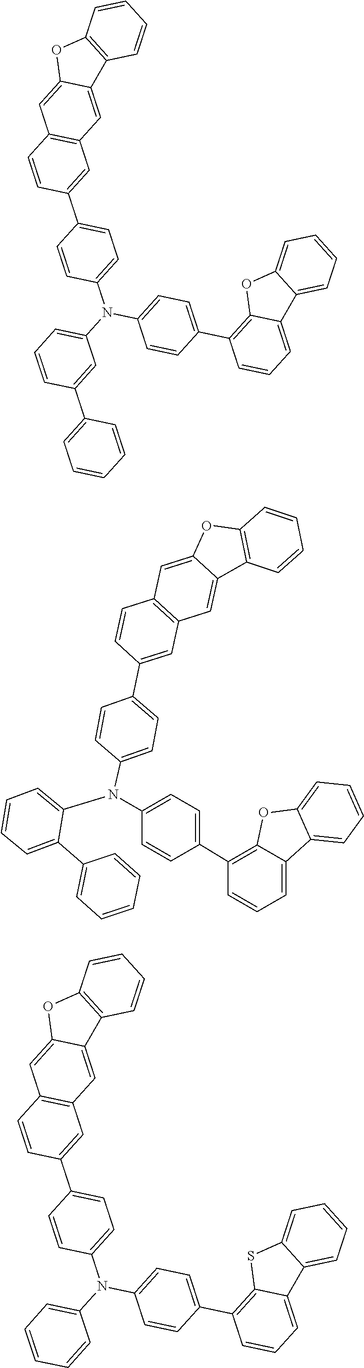

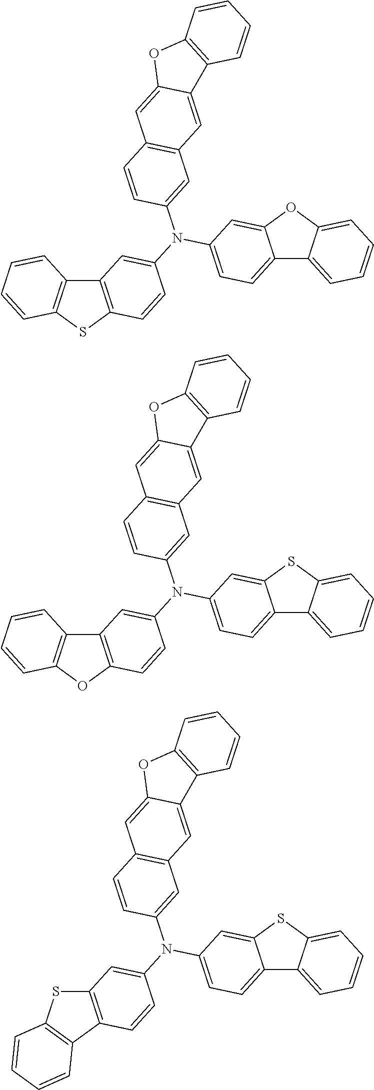 [SMILES: c1ccc2c(c1)oc1cc(N(c3ccc4cc5oc6ccccc6c5cc4c3)c3ccc4sc5ccccc5c4c3)ccc12 ; c1ccc2c(c1)oc1ccc(N(c3ccc4cc5oc6ccccc6c5cc4c3)c3ccc4c(c3)sc3ccccc34)cc12 ; c1ccc2c(c1)oc1cc3ccc(N(c4ccc5c(c4)sc4ccccc45)c4ccc5c(c4)sc4ccccc45)cc3cc12]

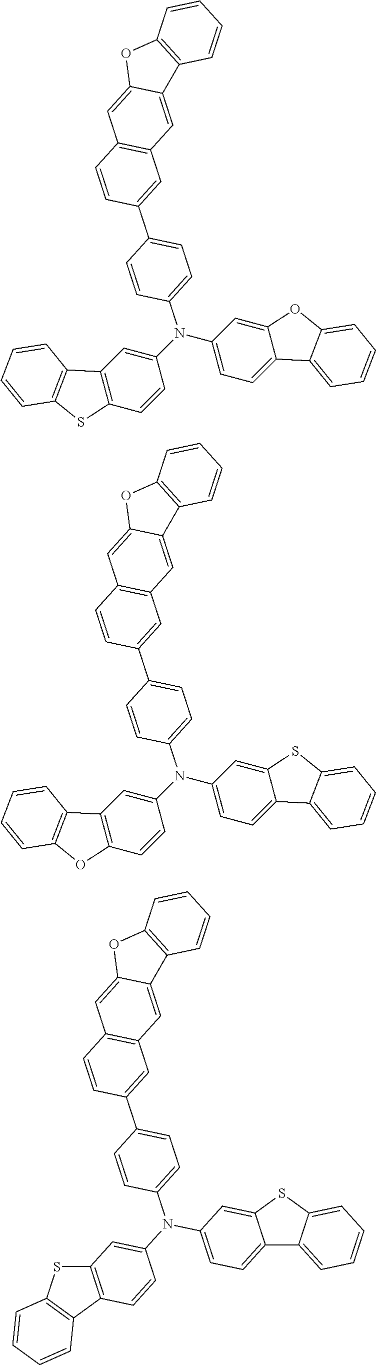

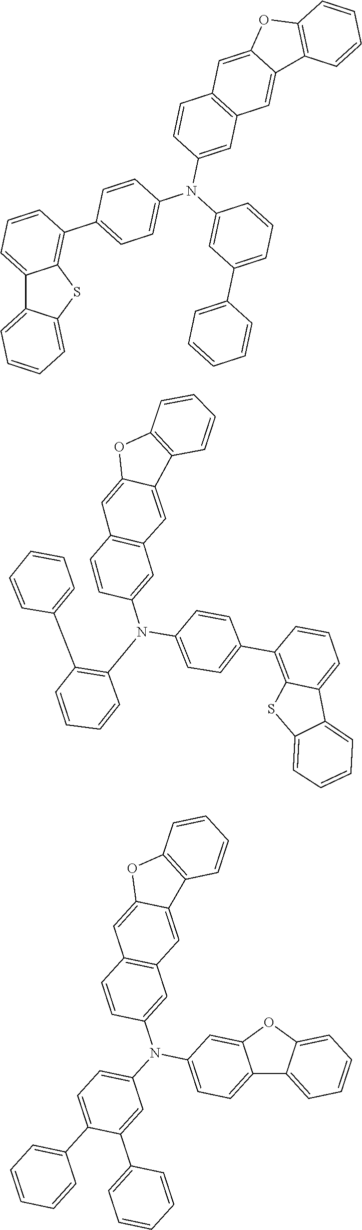

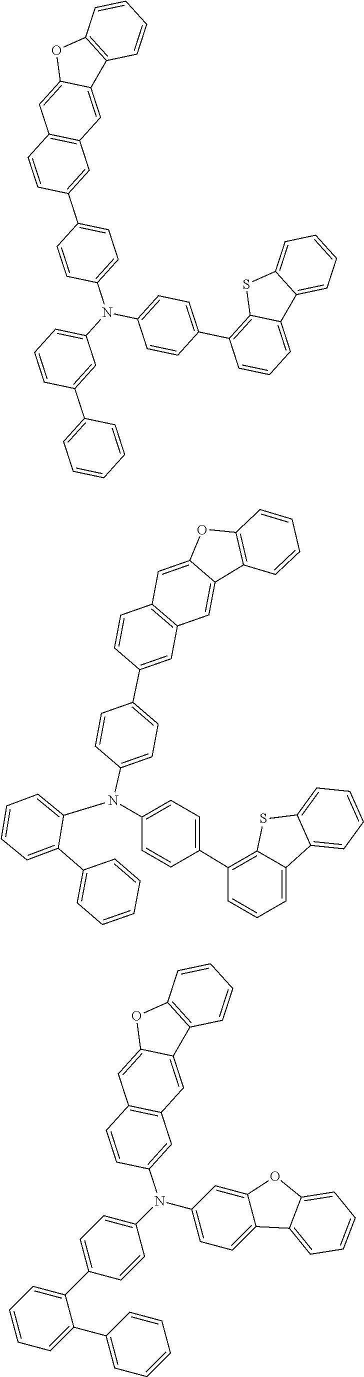 [SMILES: c1ccc(-c2cccc(N(c3ccc(-c4ccc5cc6oc7ccccc7c6cc5c4)cc3)c3ccc(-c4cccc5c4sc4ccccc45)cc3)c2)cc1 ; c1ccc(-c2ccccc2N(c2ccc(-c3ccc4cc5oc6ccccc6c5cc4c3)cc2)c2ccc(-c3cccc4c3sc3ccccc34)cc2)cc1 ; c1ccc(-c2ccccc2-c2ccc(N(c3ccc4cc5oc6ccccc6c5cc4c3)c3ccc4c(c3)oc3ccccc34)cc2)cc1]

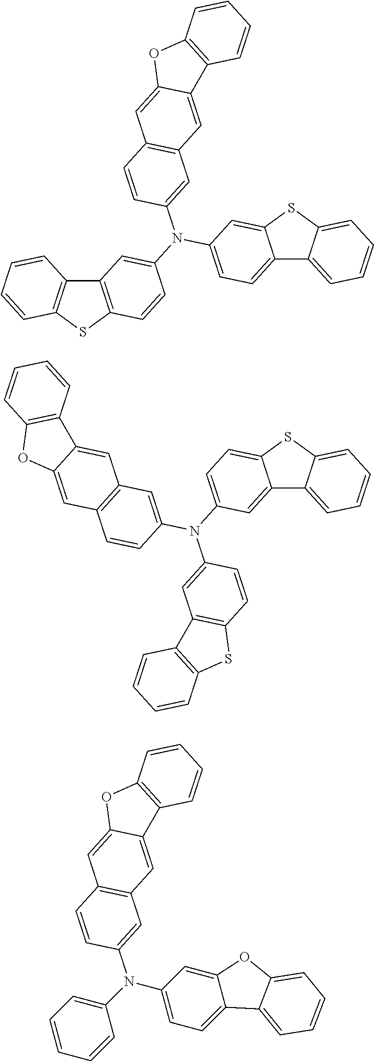

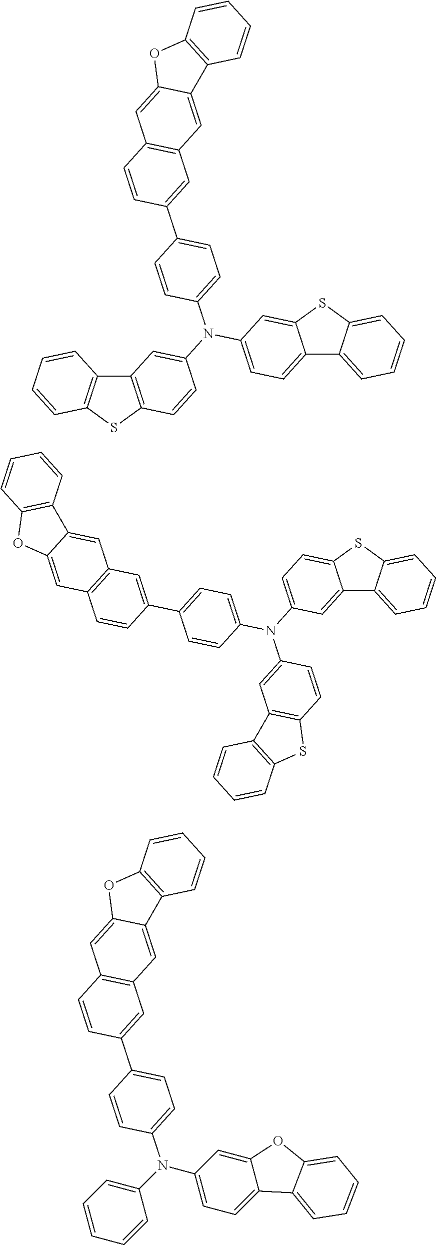

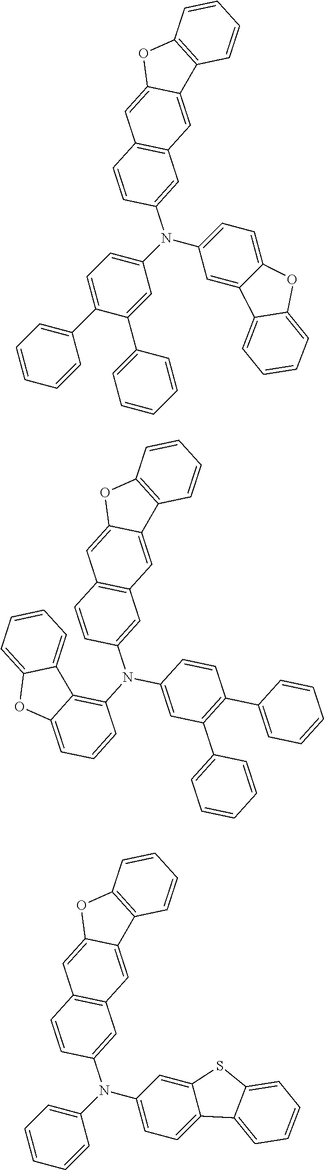

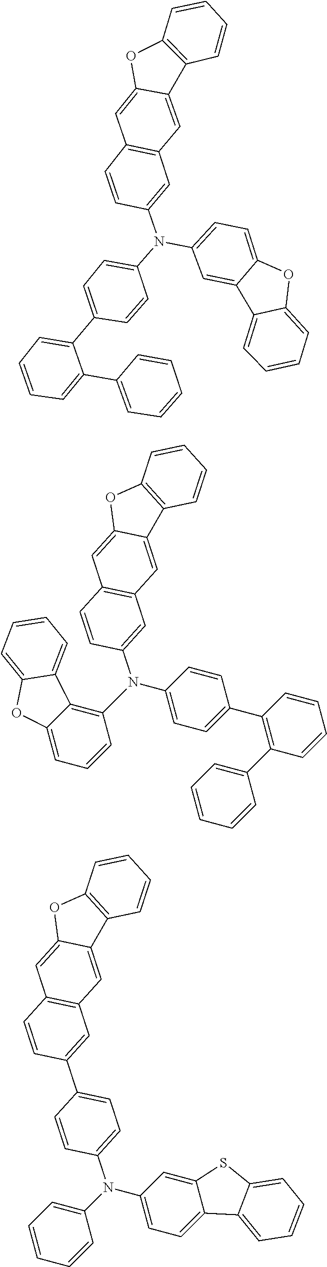

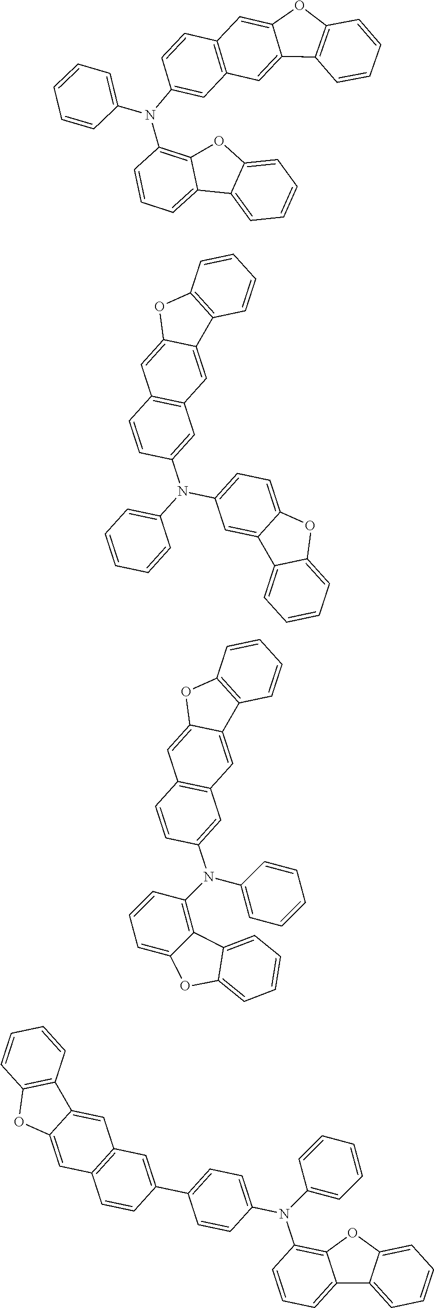

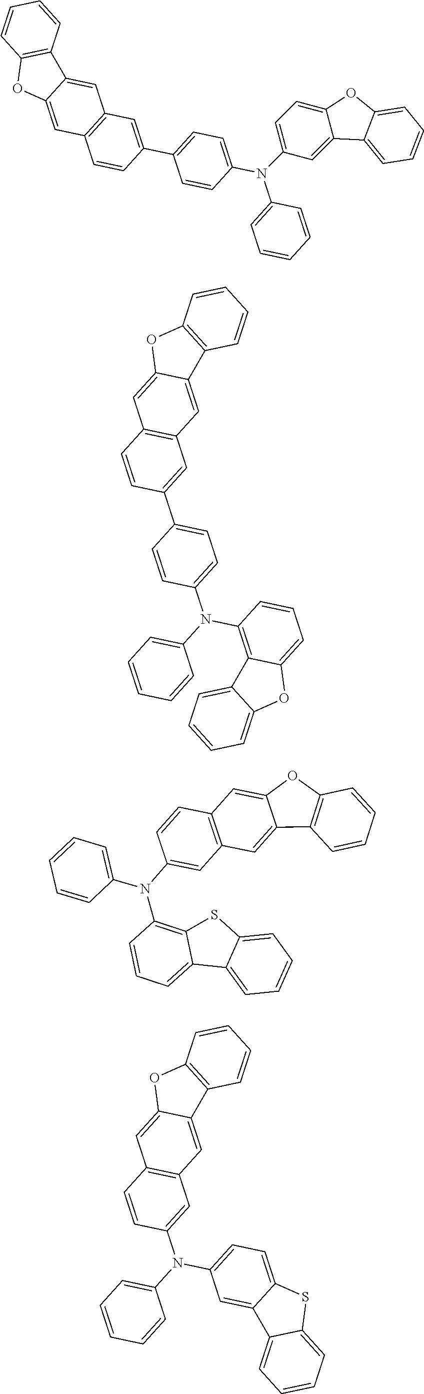

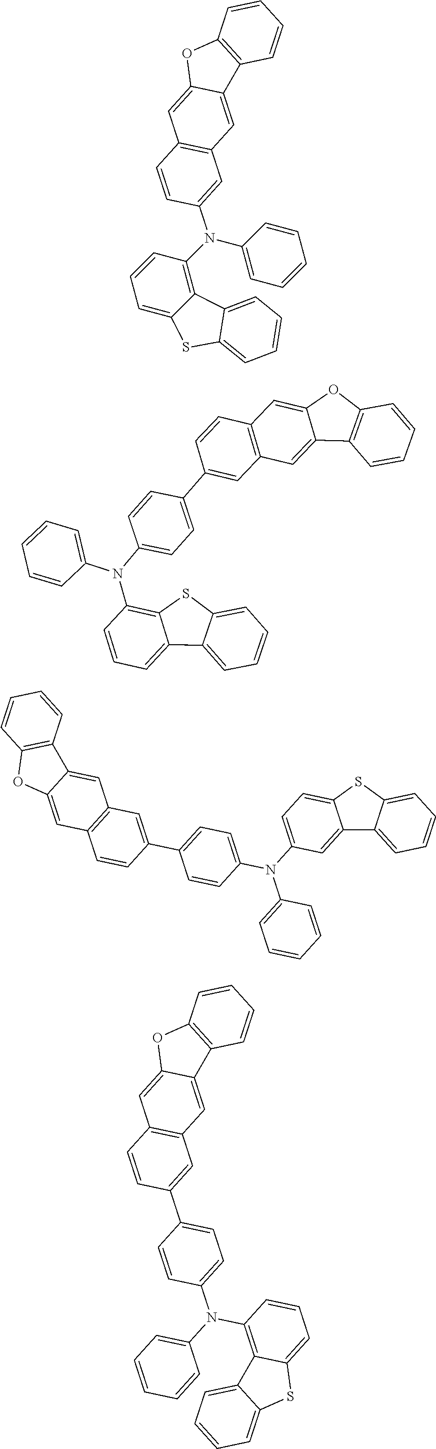 [SMILES: c1ccc(N(c2ccc3cc4oc5ccccc5c4cc3c2)c2cccc3sc4ccccc4c23)cc1 ; c1ccc(N(c2ccc(-c3ccc4cc5oc6ccccc6c5cc4c3)cc2)c2cccc3c2sc2ccccc23)cc1 ; c1ccc(N(c2ccc(-c3ccc4cc5oc6ccccc6c5cc4c3)cc2)c2ccc3sc4ccccc4c3c2)cc1 ; c1ccc(N(c2ccc(-c3ccc4cc5oc6ccccc6c5cc4c3)cc2)c2cccc3sc4ccccc4c23)cc1]

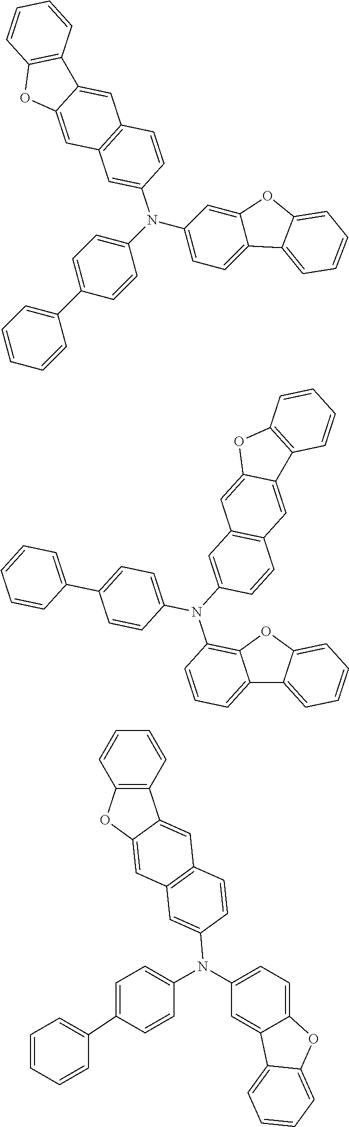 [SMILES: c1ccc(-c2ccc(N(c3ccc4cc5c(cc4c3)oc3ccccc35)c3ccc4c(c3)oc3ccccc34)cc2)cc1 ; c1ccc(-c2ccc(N(c3ccc4cc5c(cc4c3)oc3ccccc35)c3cccc4c3oc3ccccc34)cc2)cc1 ; c1ccc(-c2ccc(N(c3ccc4cc5c(cc4c3)oc3ccccc35)c3ccc4oc5ccccc5c4c3)cc2)cc1]

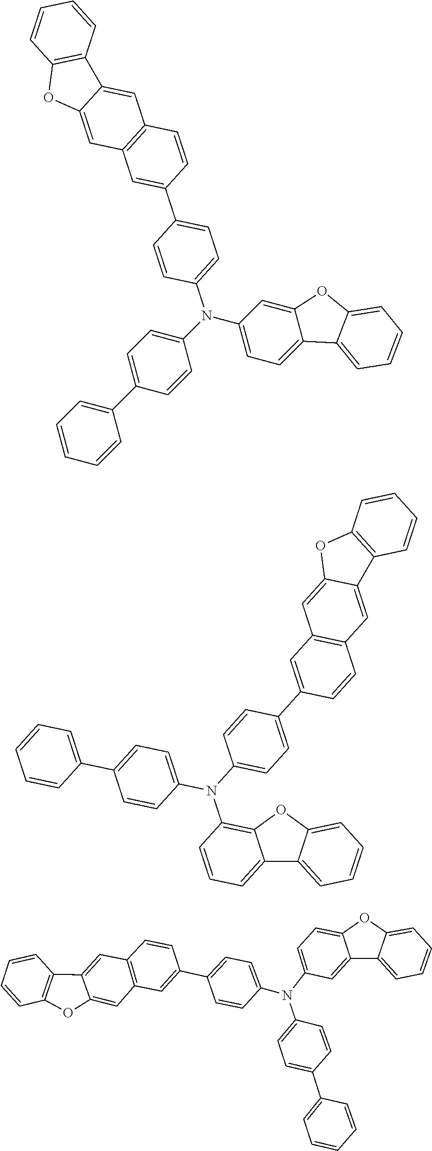

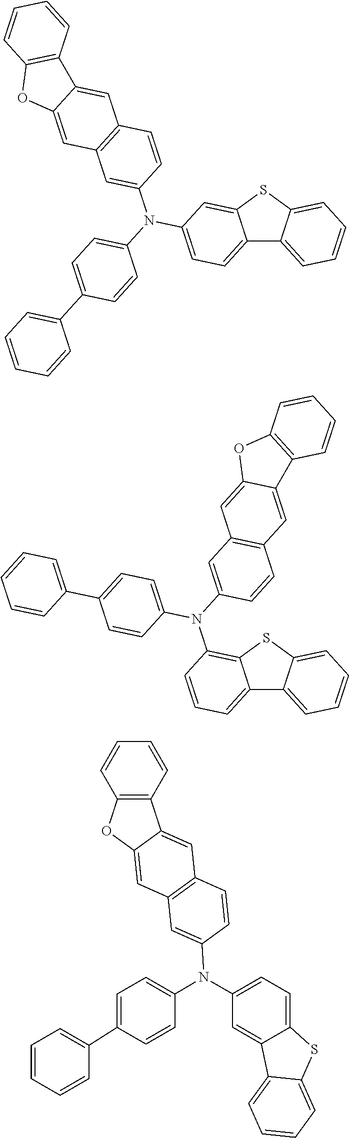 [SMILES: c1ccc(-c2ccc(N(c3ccc4cc5c(cc4c3)oc3ccccc35)c3ccc4c(c3)sc3ccccc34)cc2)cc1 ; c1ccc(-c2ccc(N(c3ccc4cc5c(cc4c3)oc3ccccc35)c3cccc4c3sc3ccccc34)cc2)cc1 ; c1ccc(-c2ccc(N(c3ccc4cc5c(cc4c3)oc3ccccc35)c3ccc4sc5ccccc5c4c3)cc2)cc1]

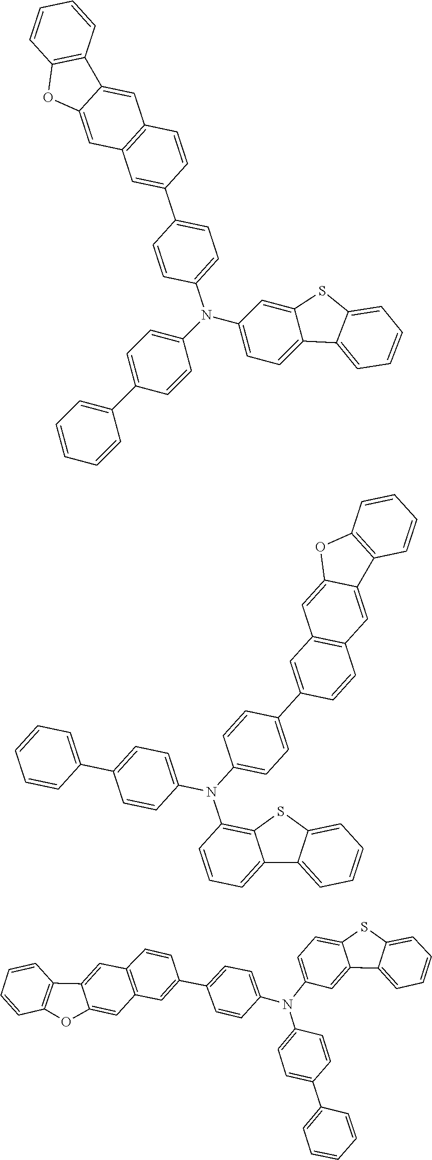 [SMILES: c1ccc(-c2ccc(N(c3ccc(-c4ccc5cc6c(cc5c4)oc4ccccc46)cc3)c3ccc4c(c3)sc3ccccc34)cc2)cc1 ; c1ccc(-c2ccc(N(c3ccc(-c4ccc5cc6c(cc5c4)oc4ccccc46)cc3)c3cccc4c3sc3ccccc34)cc2)cc1 ; c1ccc(-c2ccc(N(c3ccc(-c4ccc5cc6c(cc5c4)oc4ccccc46)cc3)c3ccc4sc5ccccc5c4c3)cc2)cc1]

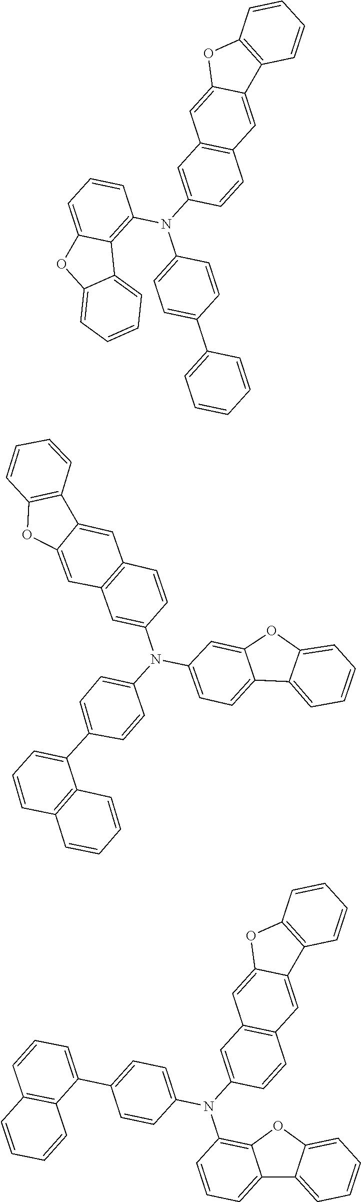 [SMILES: c1ccc(-c2ccc(N(c3ccc4cc5c(cc4c3)oc3ccccc35)c3cccc4oc5ccccc5c34)cc2)cc1 ; c1ccc2c(-c3ccc(N(c4ccc5cc6c(cc5c4)oc4ccccc46)c4ccc5c(c4)oc4ccccc45)cc3)cccc2c1 ; c1ccc2c(-c3ccc(N(c4ccc5cc6c(cc5c4)oc4ccccc46)c4cccc5c4oc4ccccc45)cc3)cccc2c1]

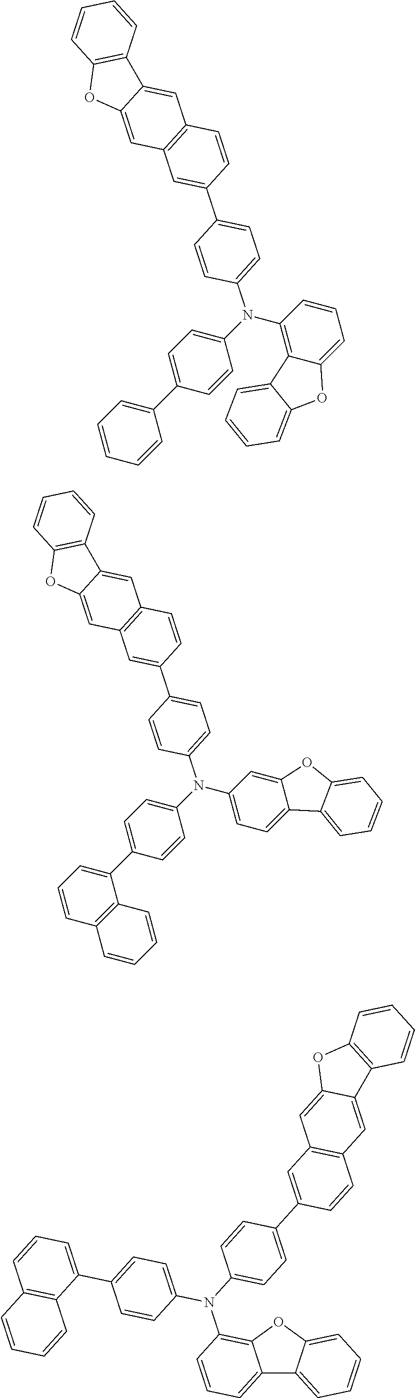 [SMILES: c1ccc(-c2ccc(N(c3ccc(-c4ccc5cc6c(cc5c4)oc4ccccc46)cc3)c3cccc4oc5ccccc5c34)cc2)cc1 ; c1ccc2c(-c3ccc(N(c4ccc(-c5ccc6cc7c(cc6c5)oc5ccccc57)cc4)c4ccc5c(c4)oc4ccccc45)cc3)cccc2c1 ; c1ccc2c(-c3ccc(N(c4ccc(-c5ccc6cc7c(cc6c5)oc5ccccc57)cc4)c4cccc5c4oc4ccccc45)cc3)cccc2c1]

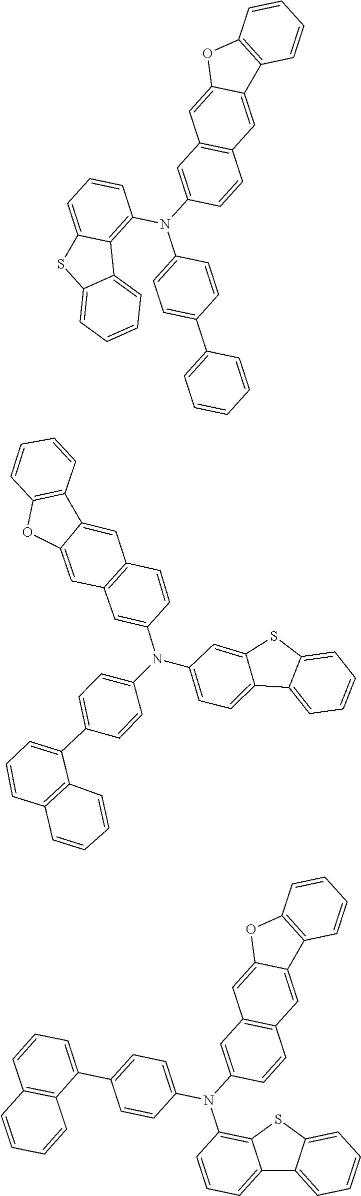 [SMILES: c1ccc(-c2ccc(N(c3ccc4cc5c(cc4c3)oc3ccccc35)c3cccc4sc5ccccc5c34)cc2)cc1 ; c1ccc2c(-c3ccc(N(c4ccc5cc6c(cc5c4)oc4ccccc46)c4ccc5c(c4)sc4ccccc45)cc3)cccc2c1 ; c1ccc2c(-c3ccc(N(c4ccc5cc6c(cc5c4)oc4ccccc46)c4cccc5c4sc4ccccc45)cc3)cccc2c1]

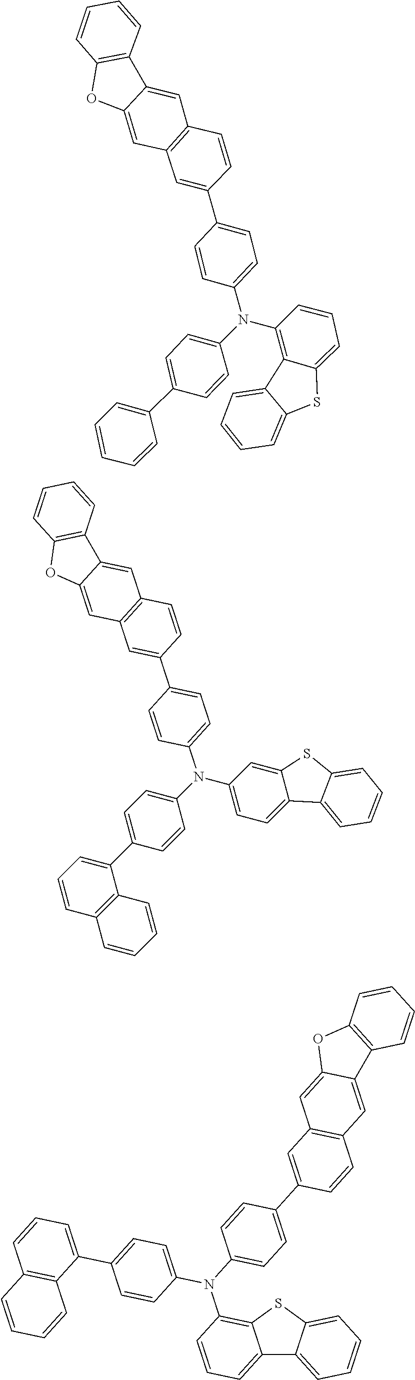

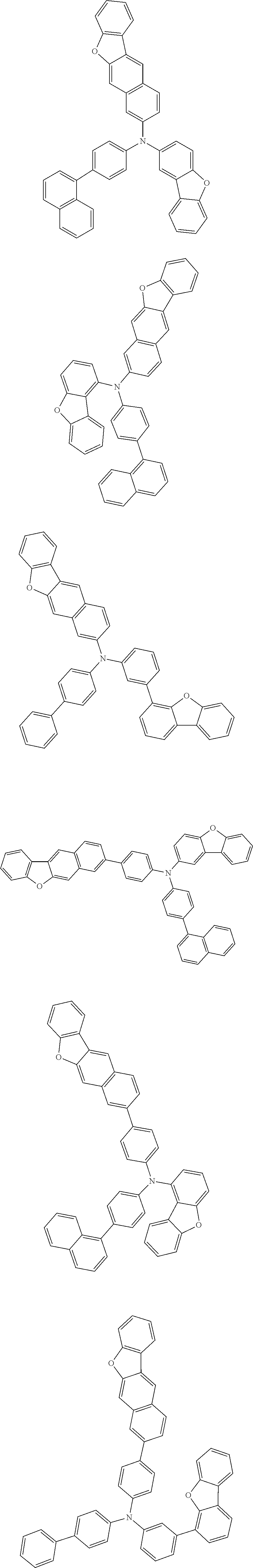

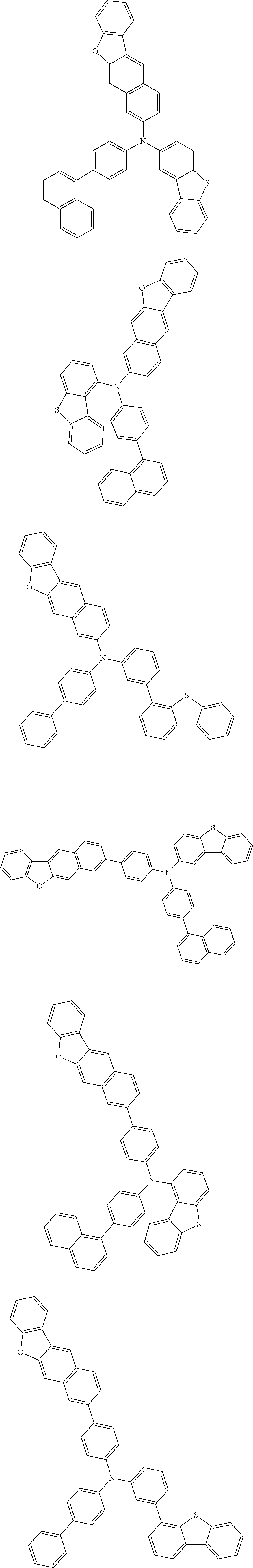

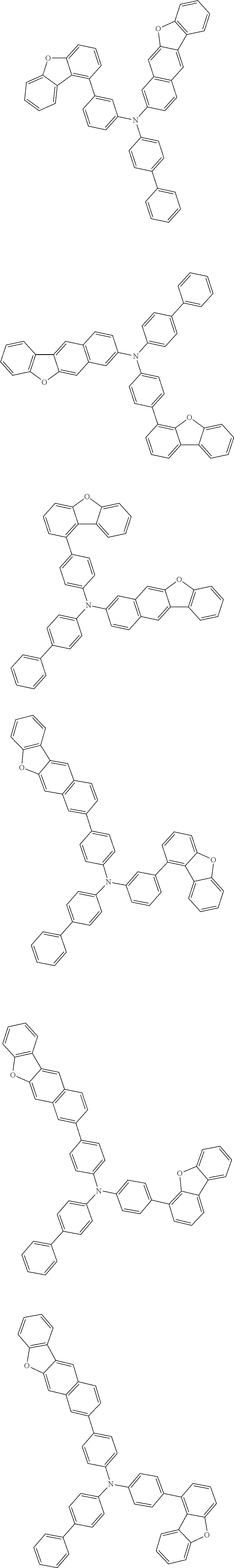

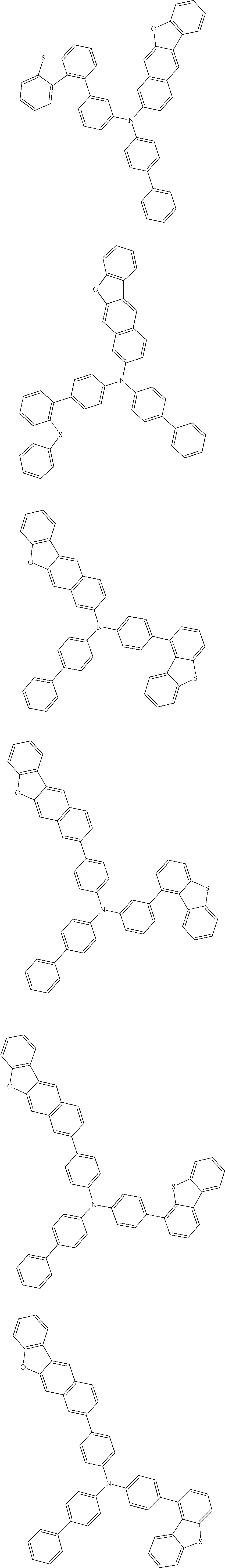

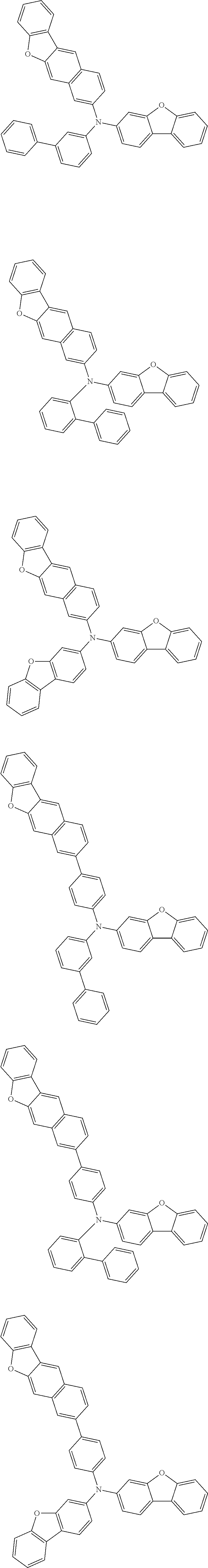 [SMILES: c1ccc(-c2cccc(N(c3ccc4cc5c(cc4c3)oc3ccccc35)c3ccc4c(c3)oc3ccccc34)c2)cc1 ; c1ccc(-c2ccccc2N(c2ccc3cc4c(cc3c2)oc2ccccc24)c2ccc3c(c2)oc2ccccc23)cc1 ; c1ccc2c(c1)oc1cc(N(c3ccc4cc5c(cc4c3)oc3ccccc35)c3ccc4c(c3)oc3ccccc34)ccc12 ; c1ccc(-c2cccc(N(c3ccc(-c4ccc5cc6c(cc5c4)oc4ccccc46)cc3)c3ccc4c(c3)oc3ccccc34)c2)cc1 ; c1ccc(-c2ccccc2N(c2ccc(-c3ccc4cc5c(cc4c3)oc3ccccc35)cc2)c2ccc3c(c2)oc2ccccc23)cc1 ; c1ccc2c(c1)oc1cc(N(c3ccc(-c4ccc5cc6c(cc5c4)oc4ccccc46)cc3)c3ccc4c(c3)oc3ccccc34)ccc12]

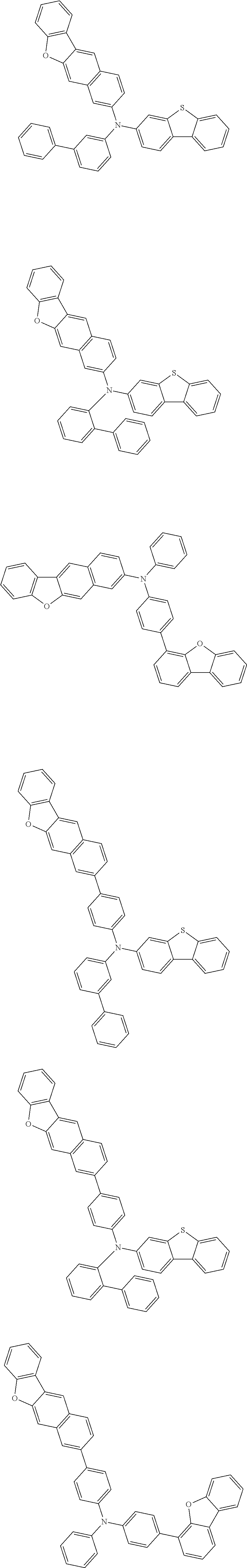 [SMILES: c1ccc(-c2cccc(N(c3ccc4cc5c(cc4c3)oc3ccccc35)c3ccc4c(c3)sc3ccccc34)c2)cc1 ; c1ccc(-c2ccccc2N(c2ccc3cc4c(cc3c2)oc2ccccc24)c2ccc3c(c2)sc2ccccc23)cc1 ; c1ccc(N(c2ccc(-c3cccc4c3oc3ccccc34)cc2)c2ccc3cc4c(cc3c2)oc2ccccc24)cc1 ; c1ccc(-c2cccc(N(c3ccc(-c4ccc5cc6c(cc5c4)oc4ccccc46)cc3)c3ccc4c(c3)sc3ccccc34)c2)cc1 ; c1ccc(-c2ccccc2N(c2ccc(-c3ccc4cc5c(cc4c3)oc3ccccc35)cc2)c2ccc3c(c2)sc2ccccc23)cc1 ; c1ccc(N(c2ccc(-c3ccc4cc5c(cc4c3)oc3ccccc35)cc2)c2ccc(-c3cccc4c3oc3ccccc34)cc2)cc1]

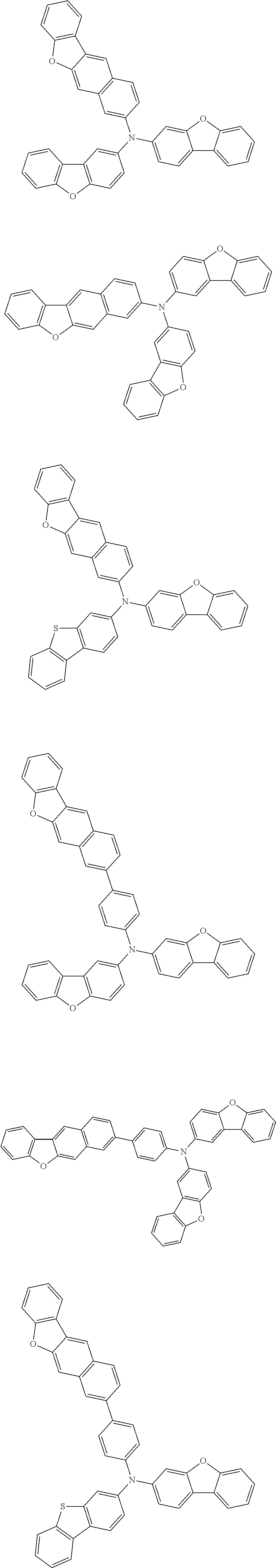 [SMILES: c1ccc2c(c1)oc1cc(N(c3ccc4cc5c(cc4c3)oc3ccccc35)c3ccc4oc5ccccc5c4c3)ccc12 ; c1ccc2c(c1)oc1ccc(N(c3ccc4cc5c(cc4c3)oc3ccccc35)c3ccc4oc5ccccc5c4c3)cc12 ; c1ccc2c(c1)oc1cc(N(c3ccc4cc5c(cc4c3)oc3ccccc35)c3ccc4c(c3)sc3ccccc34)ccc12 ; c1ccc2c(c1)oc1cc(N(c3ccc(-c4ccc5cc6c(cc5c4)oc4ccccc46)cc3)c3ccc4oc5ccccc5c4c3)ccc12 ; c1ccc2c(c1)oc1ccc(N(c3ccc(-c4ccc5cc6c(cc5c4)oc4ccccc46)cc3)c3ccc4oc5ccccc5c4c3)cc12 ; c1ccc2c(c1)oc1cc(N(c3ccc(-c4ccc5cc6c(cc5c4)oc4ccccc46)cc3)c3ccc4c(c3)sc3ccccc34)ccc12]

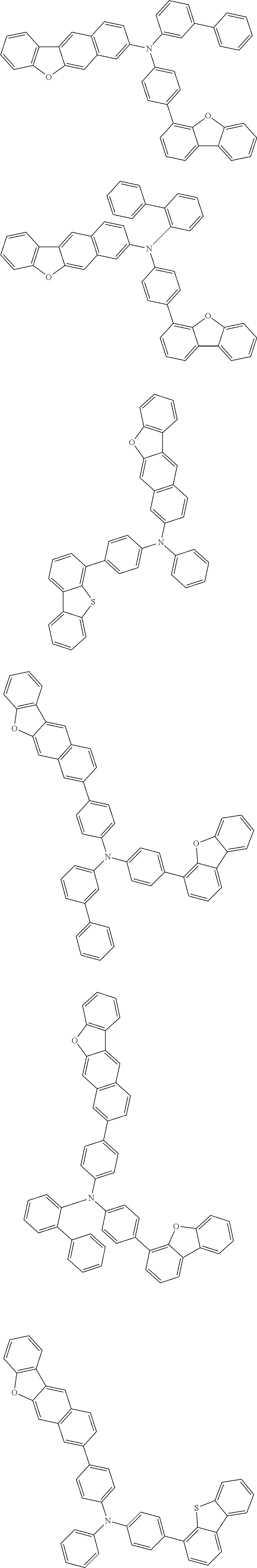

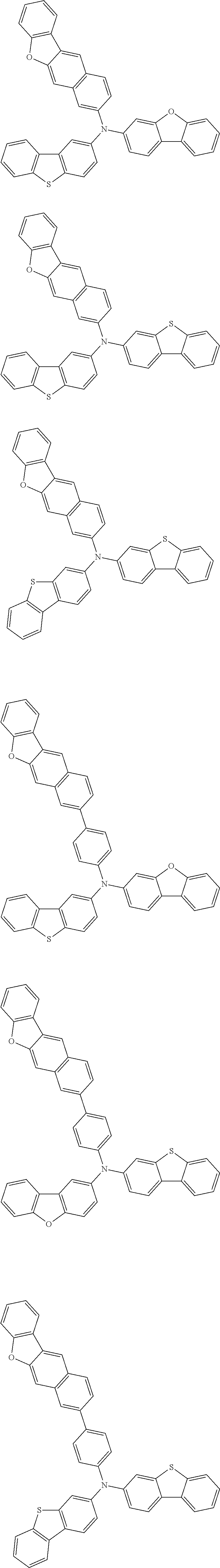 [SMILES: c1ccc2c(c1)oc1cc(N(c3ccc4cc5c(cc4c3)oc3ccccc35)c3ccc4sc5ccccc5c4c3)ccc12 ; c1ccc2c(c1)oc1cc3cc(N(c4ccc5c(c4)sc4ccccc45)c4ccc5sc6ccccc6c5c4)ccc3cc12 ; c1ccc2c(c1)oc1cc3cc(N(c4ccc5c(c4)sc4ccccc45)c4ccc5c(c4)sc4ccccc45)ccc3cc12 ; c1ccc2c(c1)oc1cc(N(c3ccc(-c4ccc5cc6c(cc5c4)oc4ccccc46)cc3)c3ccc4sc5ccccc5c4c3)ccc12 ; c1ccc2c(c1)oc1ccc(N(c3ccc(-c4ccc5cc6c(cc5c4)oc4ccccc46)cc3)c3ccc4c(c3)sc3ccccc34)cc12 ; c1ccc2c(c1)oc1cc3cc(-c4ccc(N(c5ccc6c(c5)sc5ccccc56)c5ccc6c(c5)sc5ccccc56)cc4)ccc3cc12]

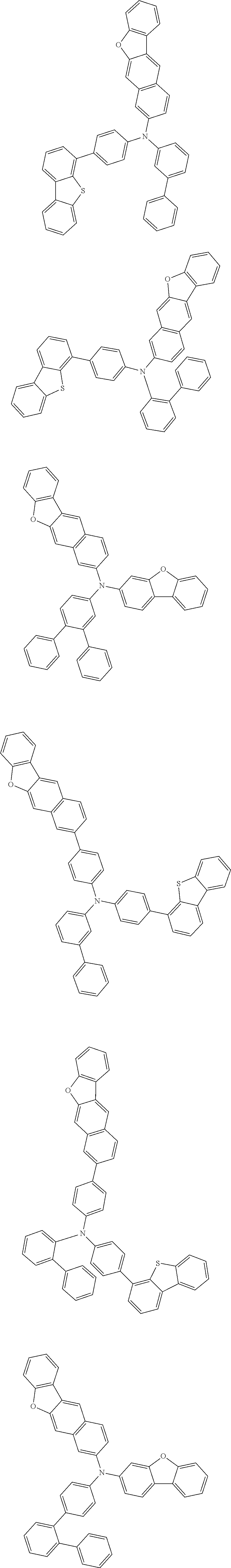

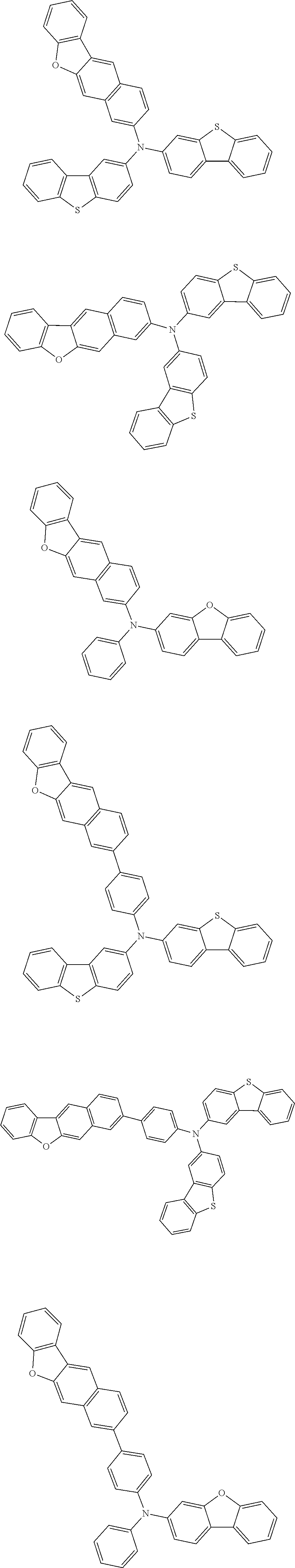 [SMILES: c1ccc2c(c1)oc1cc3cc(N(c4ccc5c(c4)sc4ccccc45)c4ccc5sc6ccccc6c5c4)ccc3cc12 ; c1ccc2c(c1)oc1cc3cc(N(c4ccc5sc6ccccc6c5c4)c4ccc5sc6ccccc6c5c4)ccc3cc12 ; c1ccc(N(c2ccc3cc4c(cc3c2)oc2ccccc24)c2ccc3c(c2)oc2ccccc23)cc1 ; c1ccc2c(c1)oc1cc3cc(-c4ccc(N(c5ccc6c(c5)sc5ccccc56)c5ccc6sc7ccccc7c6c5)cc4)ccc3cc12 ; c1ccc2c(c1)oc1cc3cc(-c4ccc(N(c5ccc6sc7ccccc7c6c5)c5ccc6sc7ccccc7c6c5)cc4)ccc3cc12 ; c1ccc(N(c2ccc(-c3ccc4cc5c(cc4c3)oc3ccccc35)cc2)c2ccc3c(c2)oc2ccccc23)cc1]

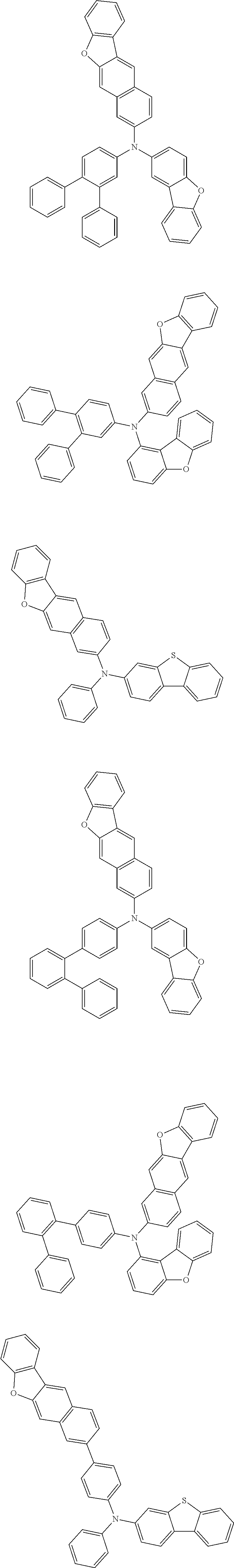 [SMILES: c1ccc(-c2ccc(N(c3ccc4cc5c(cc4c3)oc3ccccc35)c3ccc4oc5ccccc5c4c3)cc2-c2ccccc2)cc1 ; c1ccc(-c2ccc(N(c3ccc4cc5c(cc4c3)oc3ccccc35)c3cccc4oc5ccccc5c34)cc2-c2ccccc2)cc1 ; c1ccc(N(c2ccc3cc4c(cc3c2)oc2ccccc24)c2ccc3c(c2)sc2ccccc23)cc1 ; c1ccc(-c2ccccc2-c2ccc(N(c3ccc4cc5c(cc4c3)oc3ccccc35)c3ccc4oc5ccccc5c4c3)cc2)cc1 ; c1ccc(-c2ccccc2-c2ccc(N(c3ccc4cc5c(cc4c3)oc3ccccc35)c3cccc4oc5ccccc5c34)cc2)cc1 ; c1ccc(N(c2ccc(-c3ccc4cc5c(cc4c3)oc3ccccc35)cc2)c2ccc3c(c2)sc2ccccc23)cc1]

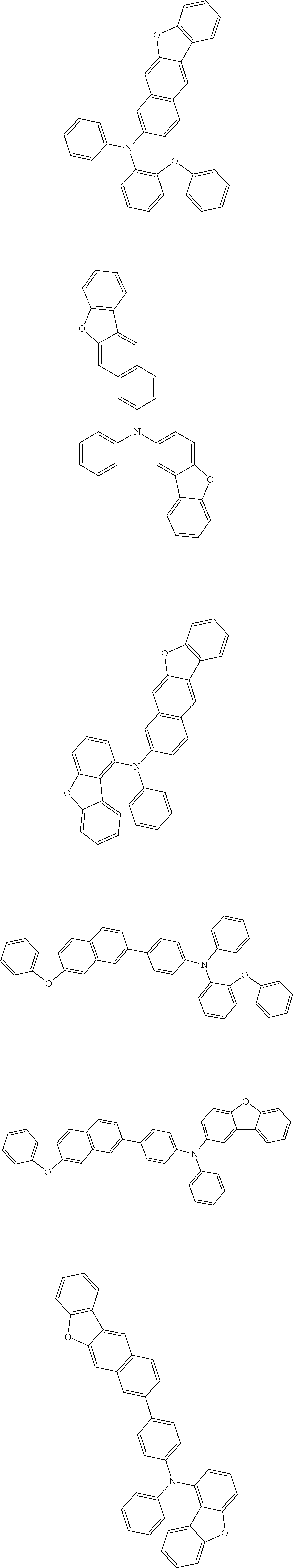 [SMILES: c1ccc(N(c2ccc3cc4c(cc3c2)oc2ccccc24)c2cccc3c2oc2ccccc23)cc1 ; c1ccc(N(c2ccc3cc4c(cc3c2)oc2ccccc24)c2ccc3oc4ccccc4c3c2)cc1 ; c1ccc(N(c2ccc3cc4c(cc3c2)oc2ccccc24)c2cccc3oc4ccccc4c23)cc1 ; c1ccc(N(c2ccc(-c3ccc4cc5c(cc4c3)oc3ccccc35)cc2)c2cccc3c2oc2ccccc23)cc1 ; c1ccc(N(c2ccc(-c3ccc4cc5c(cc4c3)oc3ccccc35)cc2)c2ccc3oc4ccccc4c3c2)cc1 ; c1ccc(N(c2ccc(-c3ccc4cc5c(cc4c3)oc3ccccc35)cc2)c2cccc3oc4ccccc4c23)cc1]

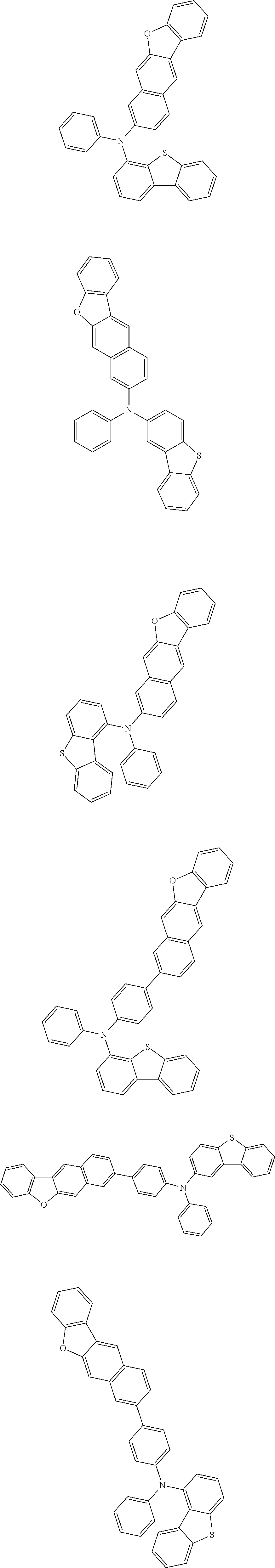 [SMILES: c1ccc(N(c2ccc3cc4c(cc3c2)oc2ccccc24)c2cccc3c2sc2ccccc23)cc1 ; c1ccc(N(c2ccc3cc4c(cc3c2)oc2ccccc24)c2ccc3sc4ccccc4c3c2)cc1 ; c1ccc(N(c2ccc3cc4c(cc3c2)oc2ccccc24)c2cccc3sc4ccccc4c23)cc1 ; c1ccc(N(c2ccc(-c3ccc4cc5c(cc4c3)oc3ccccc35)cc2)c2cccc3c2sc2ccccc23)cc1 ; c1ccc(N(c2ccc(-c3ccc4cc5c(cc4c3)oc3ccccc35)cc2)c2ccc3sc4ccccc4c3c2)cc1 ; c1ccc(N(c2ccc(-c3ccc4cc5c(cc4c3)oc3ccccc35)cc2)c2cccc3sc4ccccc4c23)cc1]

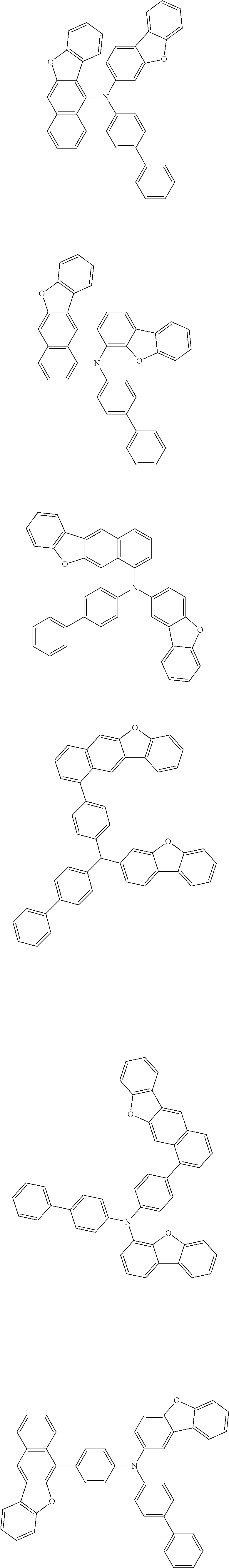 [SMILES: c1ccc(-c2ccc(N(c3ccc4c(c3)oc3ccccc34)c3c4ccccc4cc4oc5ccccc5c34)cc2)cc1 ; c1ccc(-c2ccc(N(c3cccc4cc5oc6ccccc6c5cc34)c3cccc4c3oc3ccccc34)cc2)cc1 ; c1ccc(-c2ccc(N(c3ccc4oc5ccccc5c4c3)c3cccc4cc5c(cc34)oc3ccccc35)cc2)cc1 ; c1ccc(-c2ccc(C(c3ccc(-c4cccc5cc6oc7ccccc7c6cc45)cc3)c3ccc4c(c3)oc3ccccc34)cc2)cc1 ; c1ccc(-c2ccc(N(c3ccc(-c4cccc5cc6c(cc45)oc4ccccc46)cc3)c3cccc4c3oc3ccccc34)cc2)cc1 ; c1ccc(-c2ccc(N(c3ccc(-c4c5ccccc5cc5c4oc4ccccc45)cc3)c3ccc4oc5ccccc5c4c3)cc2)cc1]

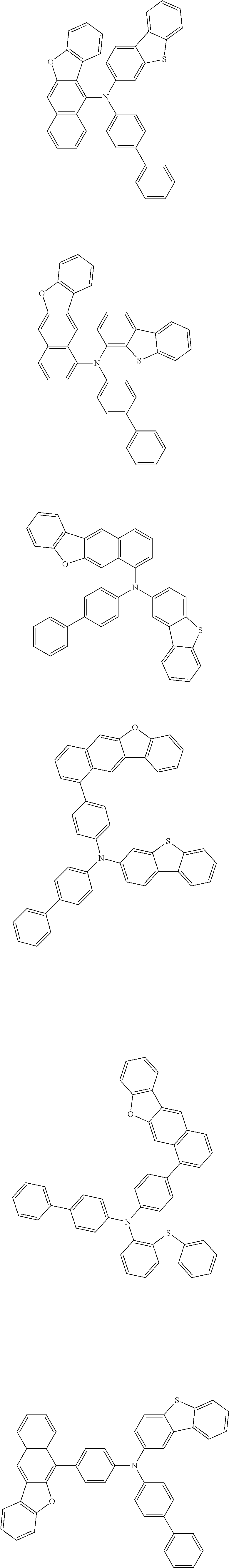 [SMILES: c1ccc(-c2ccc(N(c3ccc4c(c3)sc3ccccc34)c3c4ccccc4cc4oc5ccccc5c34)cc2)cc1 ; c1ccc(-c2ccc(N(c3cccc4cc5oc6ccccc6c5cc34)c3cccc4c3sc3ccccc34)cc2)cc1 ; c1ccc(-c2ccc(N(c3ccc4sc5ccccc5c4c3)c3cccc4cc5c(cc34)oc3ccccc35)cc2)cc1 ; c1ccc(-c2ccc(N(c3ccc(-c4cccc5cc6oc7ccccc7c6cc45)cc3)c3ccc4c(c3)sc3ccccc34)cc2)cc1 ; c1ccc(-c2ccc(N(c3ccc(-c4cccc5cc6c(cc45)oc4ccccc46)cc3)c3cccc4c3sc3ccccc34)cc2)cc1 ; c1ccc(-c2ccc(N(c3ccc(-c4c5ccccc5cc5c4oc4ccccc45)cc3)c3ccc4sc5ccccc5c4c3)cc2)cc1]

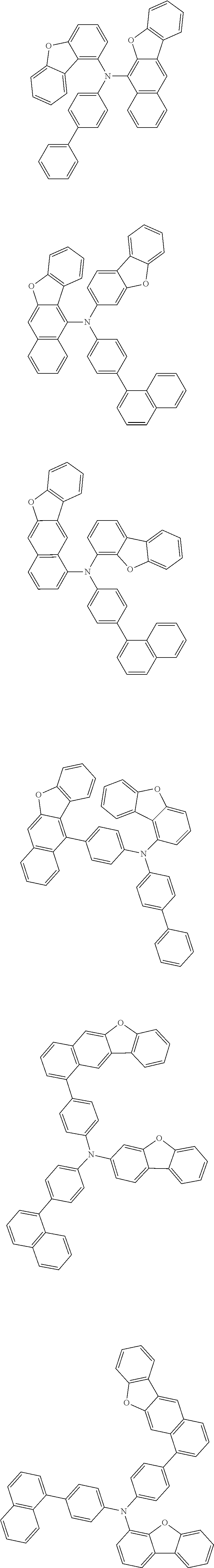 [SMILES: c1ccc(-c2ccc(N(c3c4ccccc4cc4c3oc3ccccc34)c3cccc4oc5ccccc5c34)cc2)cc1 ; c1ccc2c(-c3ccc(N(c4ccc5c(c4)oc4ccccc45)c4c5ccccc5cc5oc6ccccc6c45)cc3)cccc2c1 ; c1ccc2c(-c3ccc(N(c4cccc5cc6oc7ccccc7c6cc45)c4cccc5c4oc4ccccc45)cc3)cccc2c1 ; c1ccc(-c2ccc(N(c3ccc(-c4c5ccccc5cc5oc6ccccc6c45)cc3)c3cccc4oc5ccccc5c34)cc2)cc1 ; c1ccc2c(-c3ccc(N(c4ccc(-c5cccc6cc7oc8ccccc8c7cc56)cc4)c4ccc5c(c4)oc4ccccc45)cc3)cccc2c1 ; c1ccc2c(-c3ccc(N(c4ccc(-c5cccc6cc7c(cc56)oc5ccccc57)cc4)c4cccc5c4oc4ccccc45)cc3)cccc2c1]

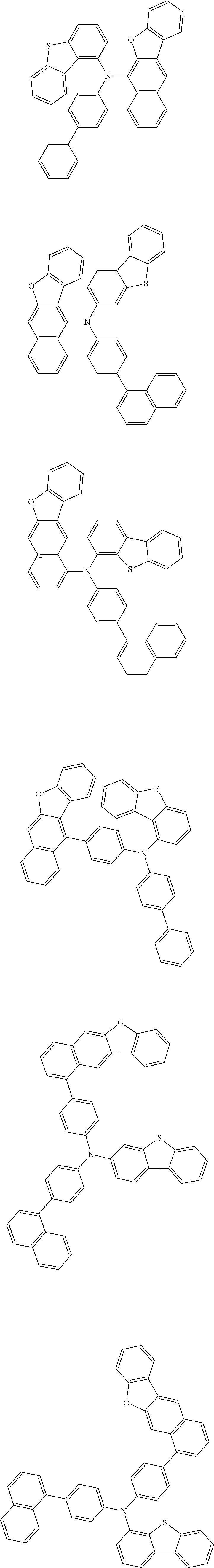 [SMILES: c1ccc(-c2ccc(N(c3c4ccccc4cc4c3oc3ccccc34)c3cccc4sc5ccccc5c34)cc2)cc1 ; c1ccc2c(-c3ccc(N(c4ccc5c(c4)sc4ccccc45)c4c5ccccc5cc5oc6ccccc6c45)cc3)cccc2c1 ; c1ccc2c(-c3ccc(N(c4cccc5cc6oc7ccccc7c6cc45)c4cccc5c4sc4ccccc45)cc3)cccc2c1 ; c1ccc(-c2ccc(N(c3ccc(-c4c5ccccc5cc5oc6ccccc6c45)cc3)c3cccc4sc5ccccc5c34)cc2)cc1 ; c1ccc2c(-c3ccc(N(c4ccc(-c5cccc6cc7oc8ccccc8c7cc56)cc4)c4ccc5c(c4)sc4ccccc45)cc3)cccc2c1 ; c1ccc2c(-c3ccc(N(c4ccc(-c5cccc6cc7c(cc56)oc5ccccc57)cc4)c4cccc5c4sc4ccccc45)cc3)cccc2c1]

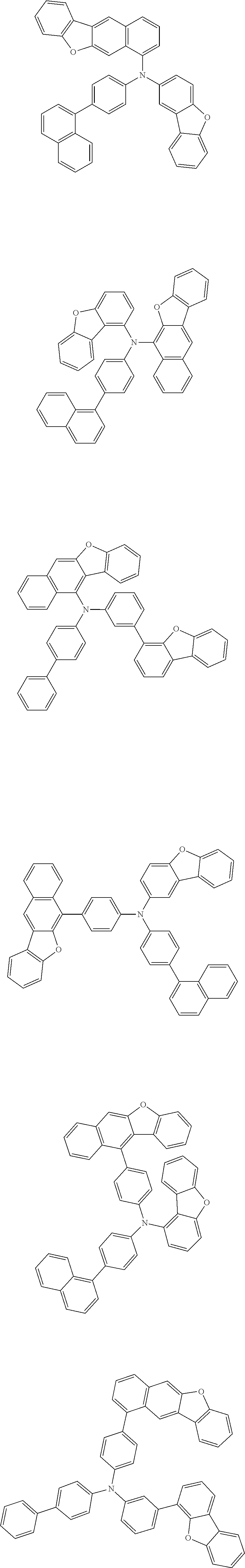

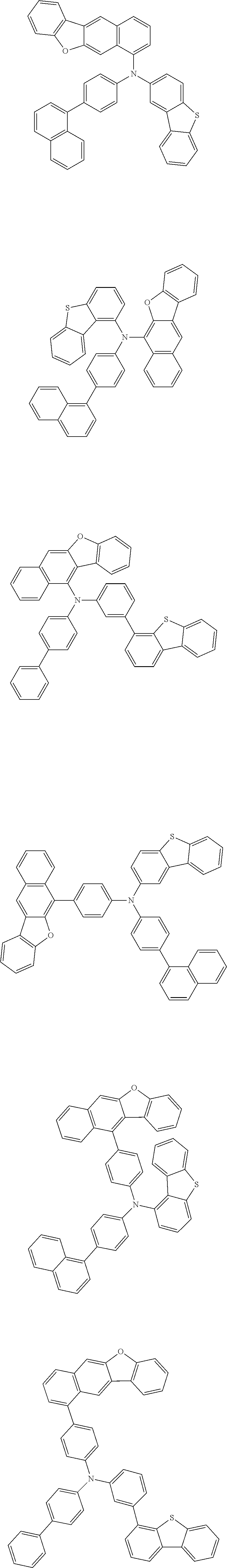

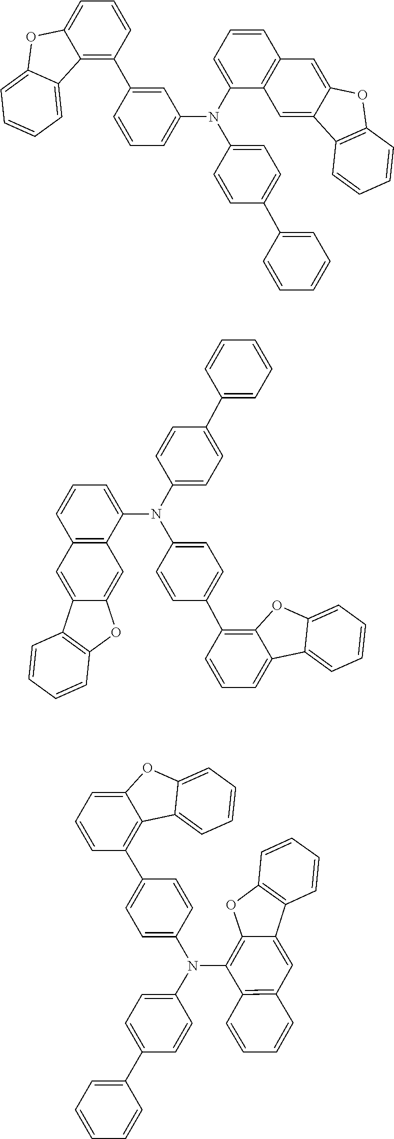 [SMILES: c1ccc(-c2ccc(N(c3cccc(-c4cccc5oc6ccccc6c45)c3)c3cccc4cc5oc6ccccc6c5cc34)cc2)cc1 ; c1ccc(-c2ccc(N(c3ccc(-c4cccc5c4oc4ccccc45)cc3)c3cccc4cc5c(cc34)oc3ccccc35)cc2)cc1 ; c1ccc(-c2ccc(N(c3ccc(-c4cccc5oc6ccccc6c45)cc3)c3c4ccccc4cc4c3oc3ccccc34)cc2)cc1]

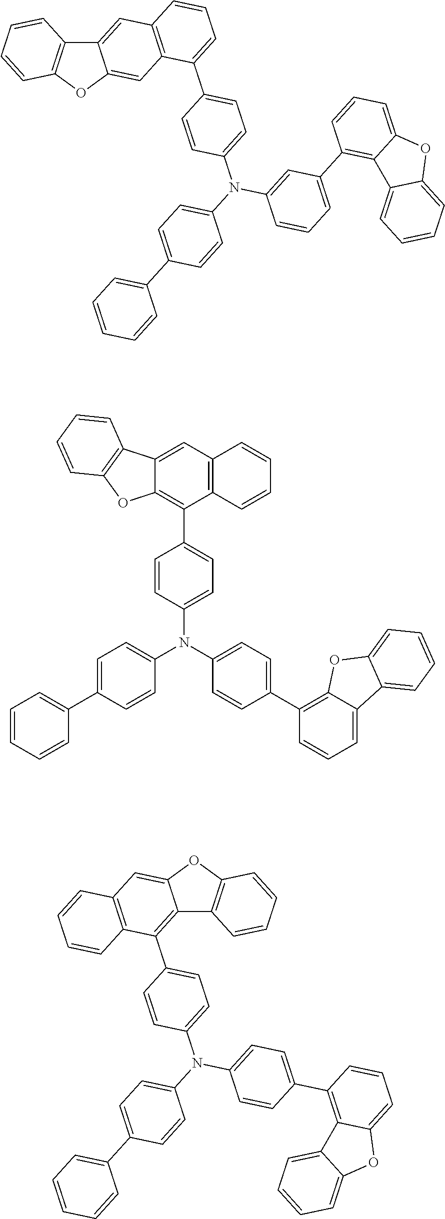 [SMILES: c1ccc(-c2ccc(N(c3ccc(-c4cccc5cc6c(cc45)oc4ccccc46)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4c5ccccc5cc5c4oc4ccccc45)cc3)cc2)cc1 ; c1ccc(-c2ccc(N(c3ccc(-c4cccc5oc6ccccc6c45)cc3)c3ccc(-c4c5ccccc5cc5oc6ccccc6c45)cc3)cc2)cc1]

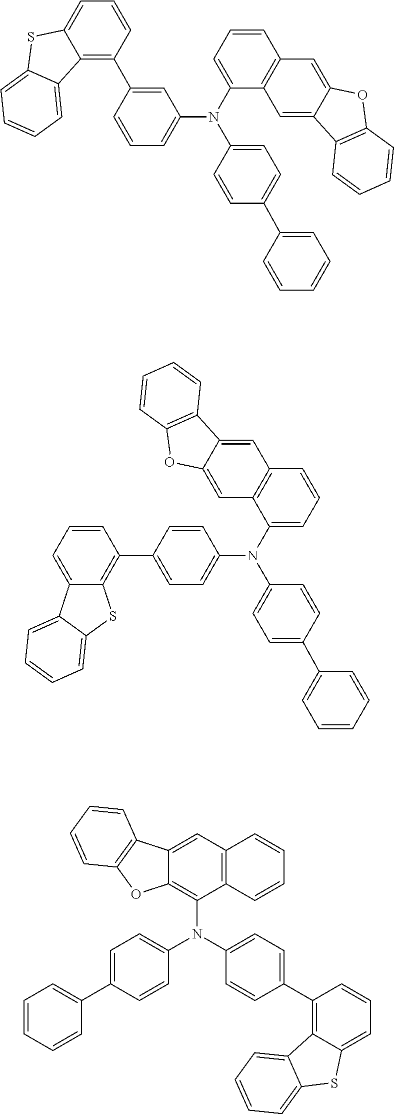 [SMILES: c1ccc(-c2ccc(N(c3cccc(-c4cccc5sc6ccccc6c45)c3)c3cccc4cc5oc6ccccc6c5cc34)cc2)cc1 ; c1ccc(-c2ccc(N(c3ccc(-c4cccc5c4sc4ccccc45)cc3)c3cccc4cc5c(cc34)oc3ccccc35)cc2)cc1 ; c1ccc(-c2ccc(N(c3ccc(-c4cccc5sc6ccccc6c45)cc3)c3c4ccccc4cc4c3oc3ccccc34)cc2)cc1]

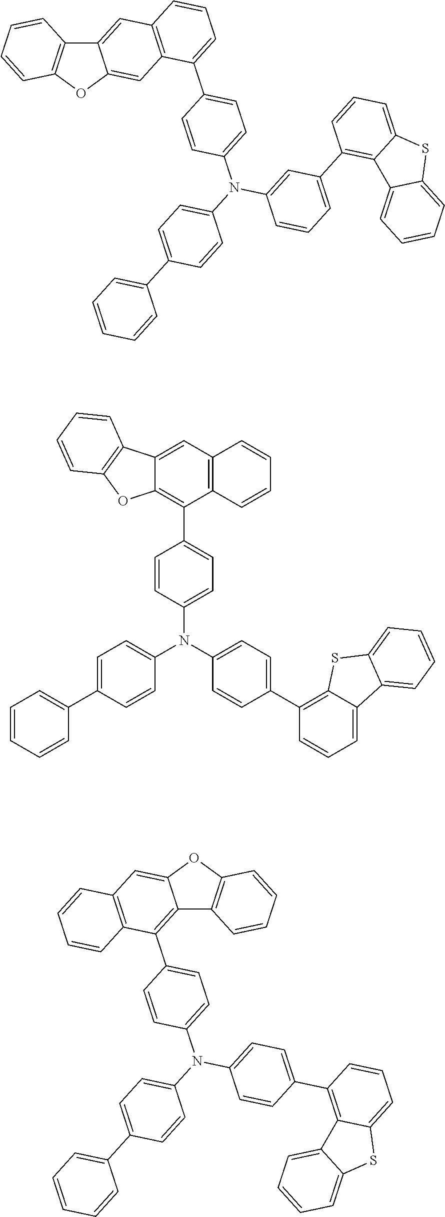 [SMILES: c1ccc(-c2ccc(N(c3ccc(-c4cccc5cc6c(cc45)oc4ccccc46)cc3)c3cccc(-c4cccc5sc6ccccc6c45)c3)cc2)cc1 ; c1ccc(-c2ccc(N(c3ccc(-c4c5ccccc5cc5c4oc4ccccc45)cc3)c3ccc(-c4cccc5c4sc4ccccc45)cc3)cc2)cc1 ; c1ccc(-c2ccc(N(c3ccc(-c4c5ccccc5cc5oc6ccccc6c45)cc3)c3ccc(-c4cccc5sc6ccccc6c45)cc3)cc2)cc1]

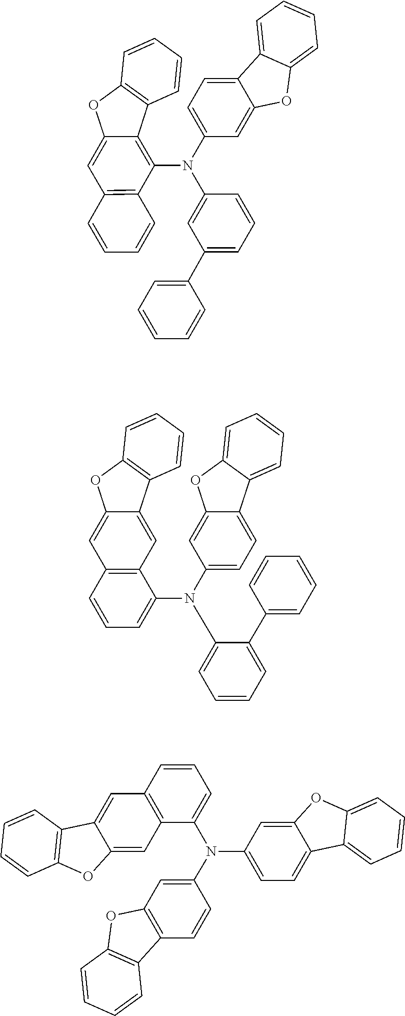 [SMILES: c1ccc(-c2cccc(N(c3ccc4c(c3)oc3ccccc34)c3c4ccccc4cc4oc5ccccc5c34)c2)cc1 ; c1ccc(-c2ccccc2N(c2ccc3c(c2)oc2ccccc23)c2cccc3cc4oc5ccccc5c4cc23)cc1 ; c1cc(N(c2ccc3c(c2)oc2ccccc23)c2ccc3c(c2)oc2ccccc23)c2cc3oc4ccccc4c3cc2c1]

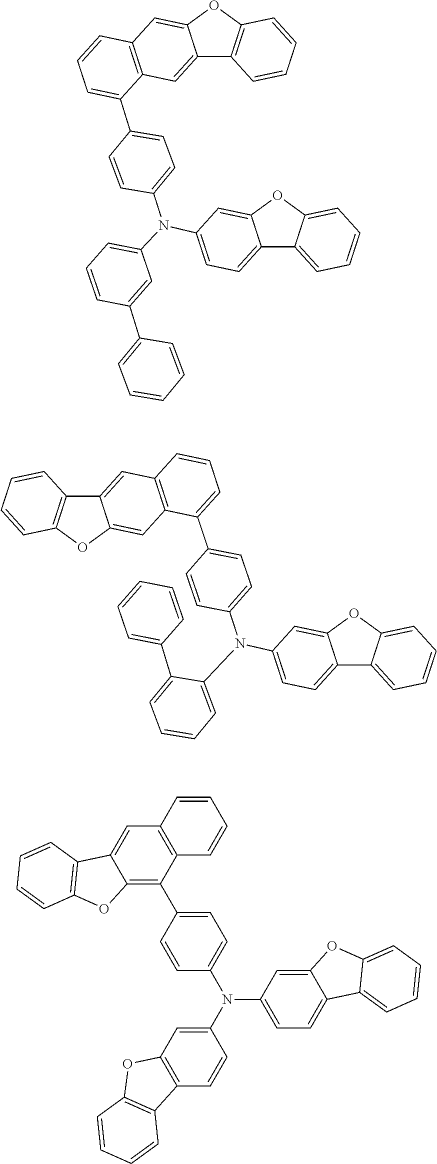 [SMILES: c1ccc(-c2cccc(N(c3ccc(-c4cccc5cc6oc7ccccc7c6cc45)cc3)c3ccc4c(c3)oc3ccccc34)c2)cc1 ; c1ccc(-c2ccccc2N(c2ccc(-c3cccc4cc5c(cc34)oc3ccccc35)cc2)c2ccc3c(c2)oc2ccccc23)cc1 ; c1ccc2c(-c3ccc(N(c4ccc5c(c4)oc4ccccc45)c4ccc5c(c4)oc4ccccc45)cc3)c3oc4ccccc4c3cc2c1]

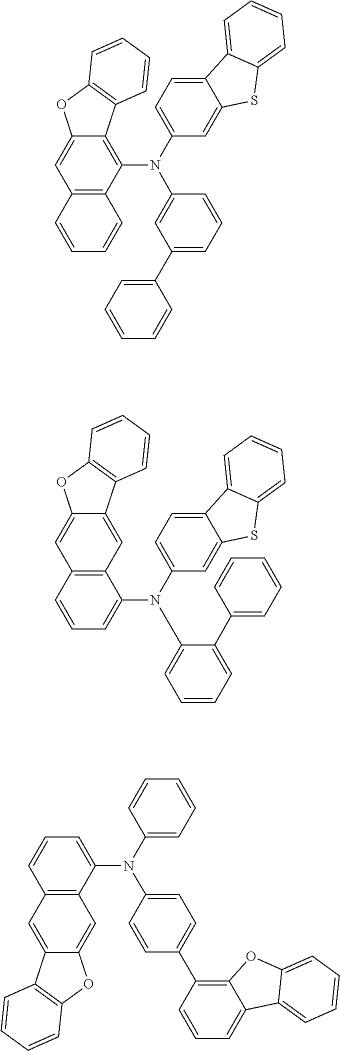 [SMILES: c1ccc(-c2cccc(N(c3ccc4c(c3)sc3ccccc34)c3c4ccccc4cc4oc5ccccc5c34)c2)cc1 ; c1ccc(-c2ccccc2N(c2ccc3c(c2)sc2ccccc23)c2cccc3cc4oc5ccccc5c4cc23)cc1 ; c1ccc(N(c2ccc(-c3cccc4c3oc3ccccc34)cc2)c2cccc3cc4c(cc23)oc2ccccc24)cc1]

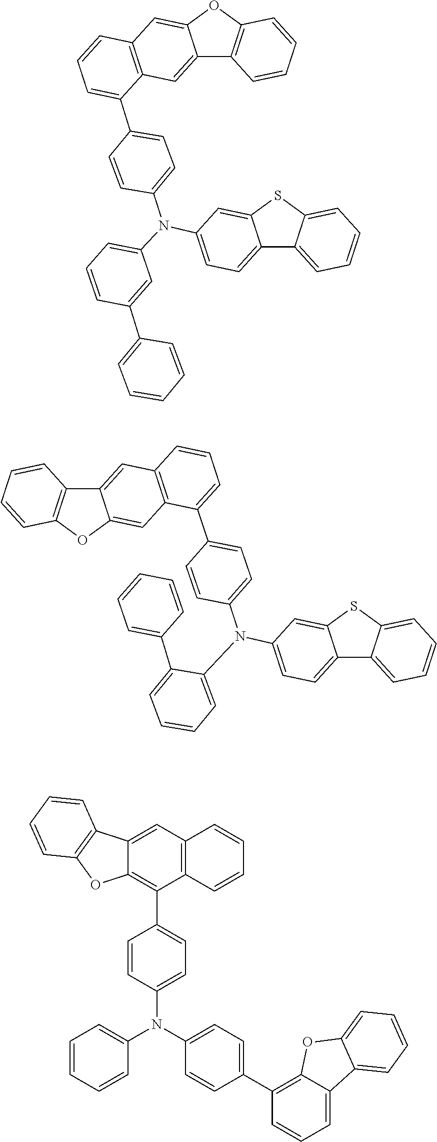 [SMILES: c1ccc(-c2cccc(N(c3ccc(-c4cccc5cc6oc7ccccc7c6cc45)cc3)c3ccc4c(c3)sc3ccccc34)c2)cc1 ; c1ccc(-c2ccccc2N(c2ccc(-c3cccc4cc5c(cc34)oc3ccccc35)cc2)c2ccc3c(c2)sc2ccccc23)cc1 ; c1ccc(N(c2ccc(-c3cccc4c3oc3ccccc34)cc2)c2ccc(-c3c4ccccc4cc4c3oc3ccccc34)cc2)cc1]

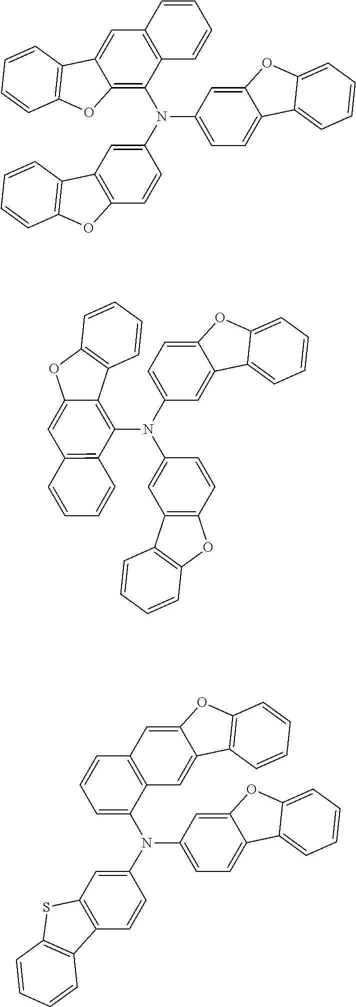 [SMILES: c1ccc2c(N(c3ccc4c(c3)oc3ccccc34)c3ccc4oc5ccccc5c4c3)c3oc4ccccc4c3cc2c1 ; c1ccc2c(N(c3ccc4oc5ccccc5c4c3)c3ccc4oc5ccccc5c4c3)c3c(cc2c1)oc1ccccc13 ; c1cc(N(c2ccc3c(c2)oc2ccccc23)c2ccc3c(c2)sc2ccccc23)c2cc3c(cc2c1)oc1ccccc13]

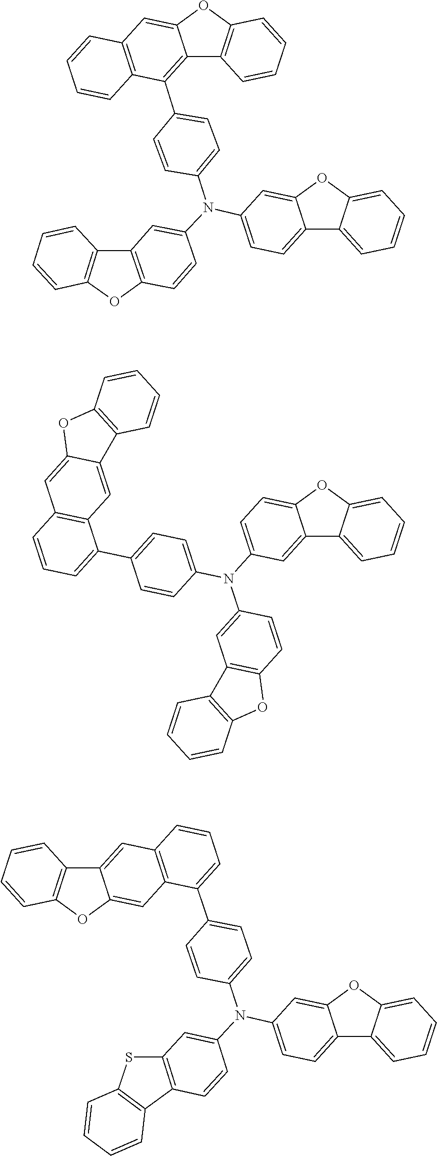 [SMILES: c1ccc2c(-c3ccc(N(c4ccc5c(c4)oc4ccccc45)c4ccc5oc6ccccc6c5c4)cc3)c3c(cc2c1)oc1ccccc13 ; c1cc(-c2ccc(N(c3ccc4oc5ccccc5c4c3)c3ccc4oc5ccccc5c4c3)cc2)c2cc3c(cc2c1)oc1ccccc13 ; c1cc(-c2ccc(N(c3ccc4c(c3)oc3ccccc34)c3ccc4c(c3)sc3ccccc34)cc2)c2cc3oc4ccccc4c3cc2c1]

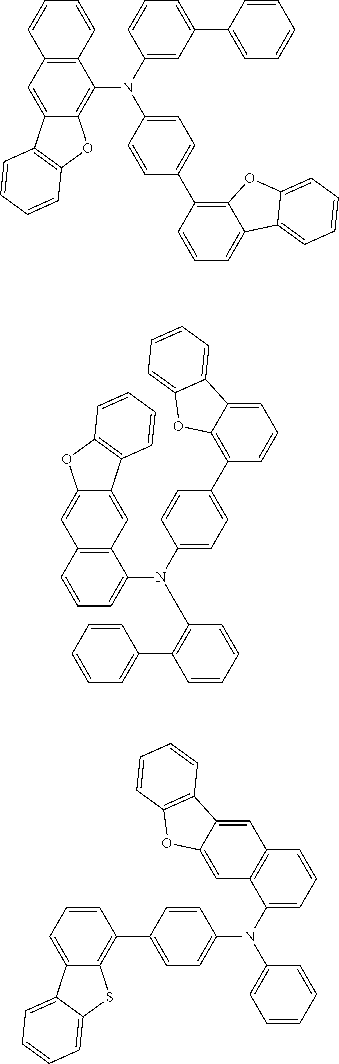 [SMILES: c1ccc(-c2cccc(N(c3ccc(-c4cccc5c4oc4ccccc45)cc3)c3c4ccccc4cc4c3oc3ccccc34)c2)cc1 ; c1ccc(-c2ccccc2N(c2ccc(-c3cccc4c3oc3ccccc34)cc2)c2cccc3cc4oc5ccccc5c4cc23)cc1 ; c1ccc(N(c2ccc(-c3cccc4c3sc3ccccc34)cc2)c2cccc3cc4c(cc23)oc2ccccc24)cc1]

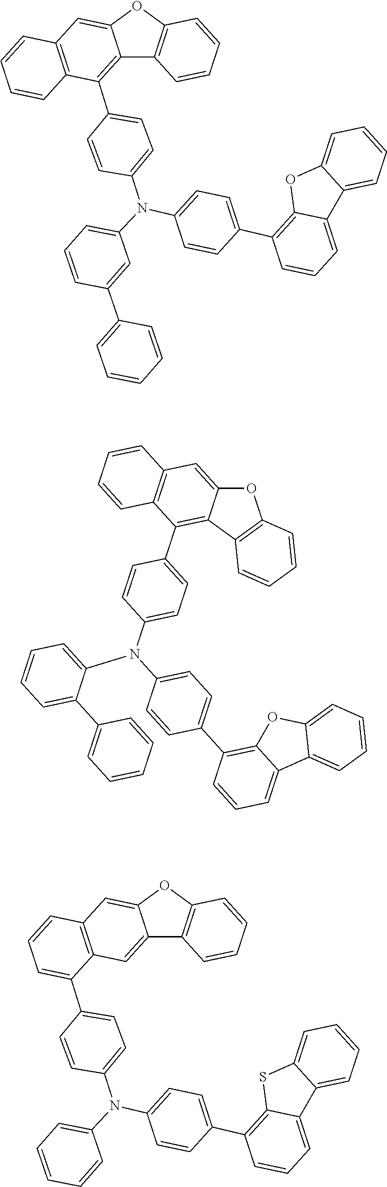 [SMILES: c1ccc(-c2cccc(N(c3ccc(-c4cccc5c4oc4ccccc45)cc3)c3ccc(-c4c5ccccc5cc5oc6ccccc6c45)cc3)c2)cc1 ; c1ccc(-c2ccccc2N(c2ccc(-c3cccc4c3oc3ccccc34)cc2)c2ccc(-c3c4ccccc4cc4oc5ccccc5c34)cc2)cc1 ; c1ccc(N(c2ccc(-c3cccc4cc5oc6ccccc6c5cc34)cc2)c2ccc(-c3cccc4c3sc3ccccc34)cc2)cc1]

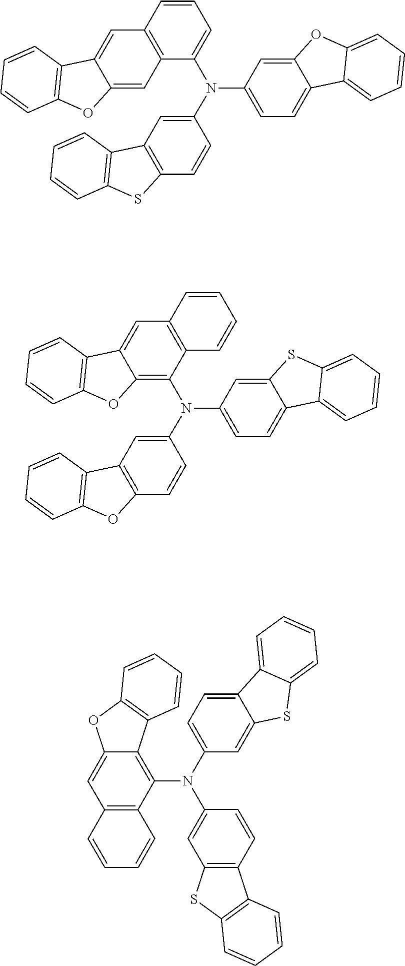 [SMILES: c1cc(N(c2ccc3c(c2)oc2ccccc23)c2ccc3sc4ccccc4c3c2)c2cc3oc4ccccc4c3cc2c1 ; c1ccc2c(N(c3ccc4c(c3)sc3ccccc34)c3ccc4oc5ccccc5c4c3)c3oc4ccccc4c3cc2c1 ; c1ccc2c(N(c3ccc4c(c3)sc3ccccc34)c3ccc4c(c3)sc3ccccc34)c3c(cc2c1)oc1ccccc13]

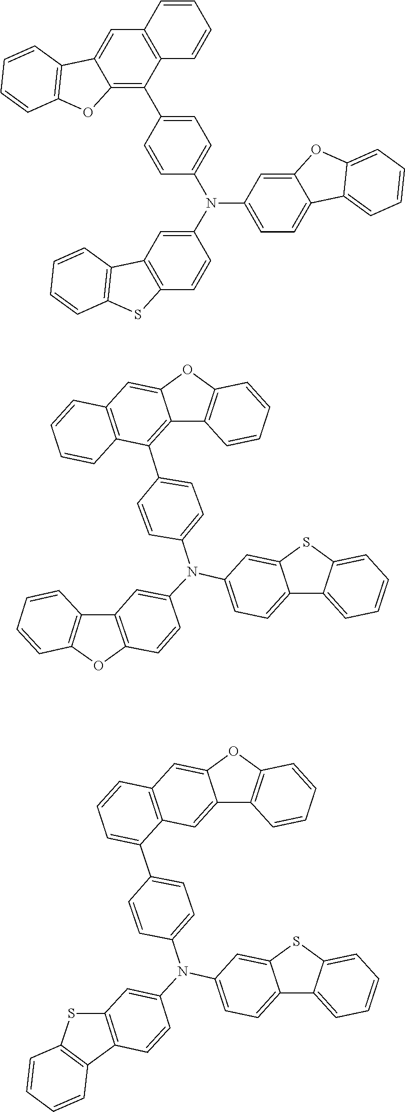 [SMILES: c1ccc2c(-c3ccc(N(c4ccc5c(c4)oc4ccccc45)c4ccc5sc6ccccc6c5c4)cc3)c3oc4ccccc4c3cc2c1 ; c1ccc2c(-c3ccc(N(c4ccc5c(c4)sc4ccccc45)c4ccc5oc6ccccc6c5c4)cc3)c3c(cc2c1)oc1ccccc13 ; c1cc(-c2ccc(N(c3ccc4c(c3)sc3ccccc34)c3ccc4c(c3)sc3ccccc34)cc2)c2cc3c(cc2c1)oc1ccccc13]

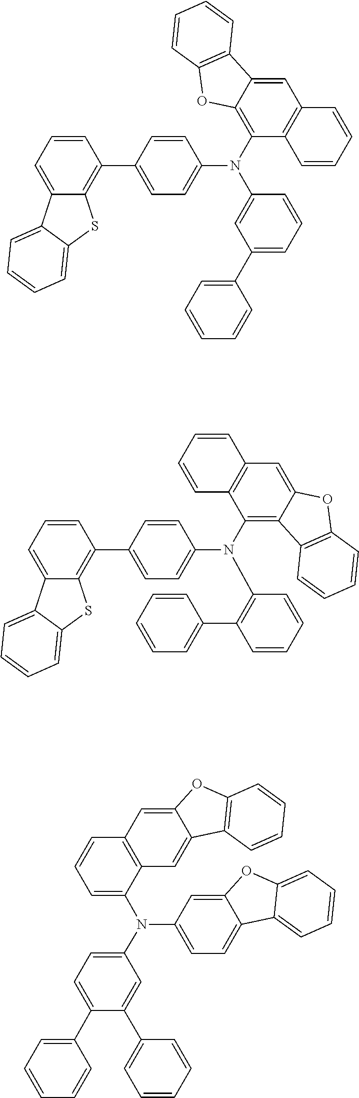 [SMILES: c1ccc(-c2cccc(N(c3ccc(-c4cccc5c4sc4ccccc45)cc3)c3c4ccccc4cc4c3oc3ccccc34)c2)cc1 ; c1ccc(-c2ccccc2N(c2ccc(-c3cccc4c3sc3ccccc34)cc2)c2c3ccccc3cc3oc4ccccc4c23)cc1 ; c1ccc(-c2ccc(N(c3ccc4c(c3)oc3ccccc34)c3cccc4cc5oc6ccccc6c5cc34)cc2-c2ccccc2)cc1]

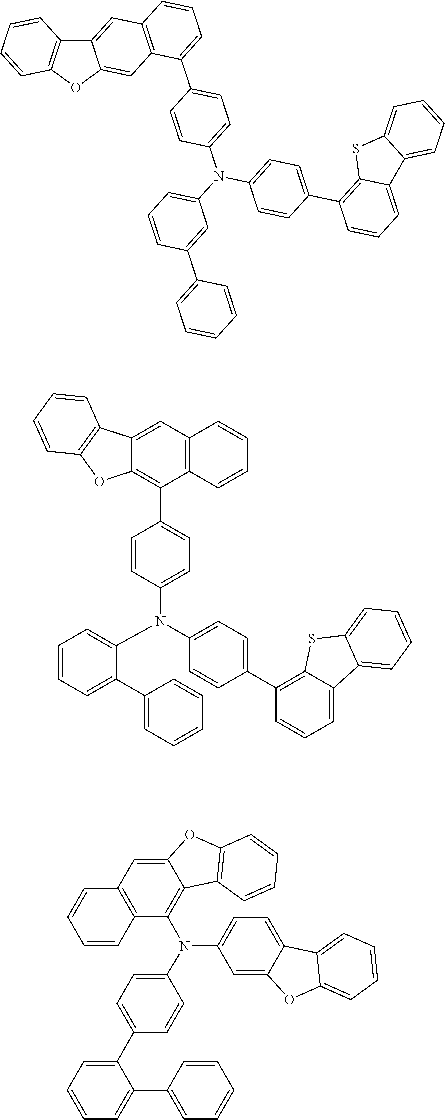 [SMILES: c1ccc(-c2cccc(N(c3ccc(-c4cccc5cc6c(cc45)oc4ccccc46)cc3)c3ccc(-c4cccc5c4sc4ccccc45)cc3)c2)cc1 ; c1ccc(-c2ccccc2N(c2ccc(-c3c4ccccc4cc4c3oc3ccccc34)cc2)c2ccc(-c3cccc4c3sc3ccccc34)cc2)cc1 ; c1ccc(-c2ccccc2-c2ccc(N(c3ccc4c(c3)oc3ccccc34)c3c4ccccc4cc4oc5ccccc5c34)cc2)cc1]

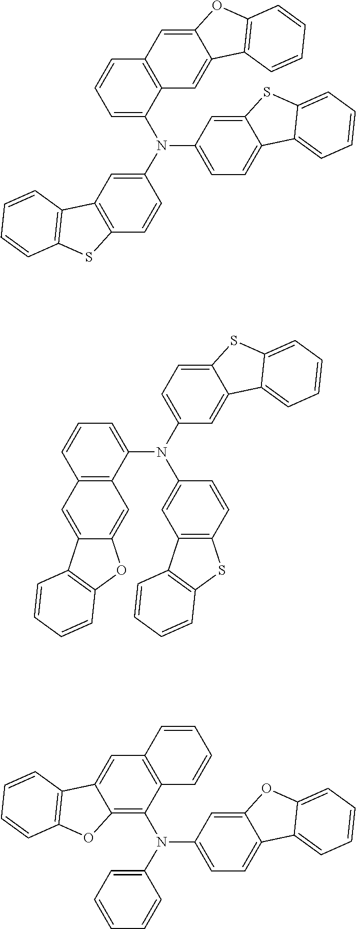 [SMILES: c1cc(N(c2ccc3c(c2)sc2ccccc23)c2ccc3sc4ccccc4c3c2)c2cc3c(cc2c1)oc1ccccc13 ; c1cc(N(c2ccc3sc4ccccc4c3c2)c2ccc3sc4ccccc4c3c2)c2cc3oc4ccccc4c3cc2c1 ; c1ccc(N(c2ccc3c(c2)oc2ccccc23)c2c3ccccc3cc3c2oc2ccccc23)cc1]

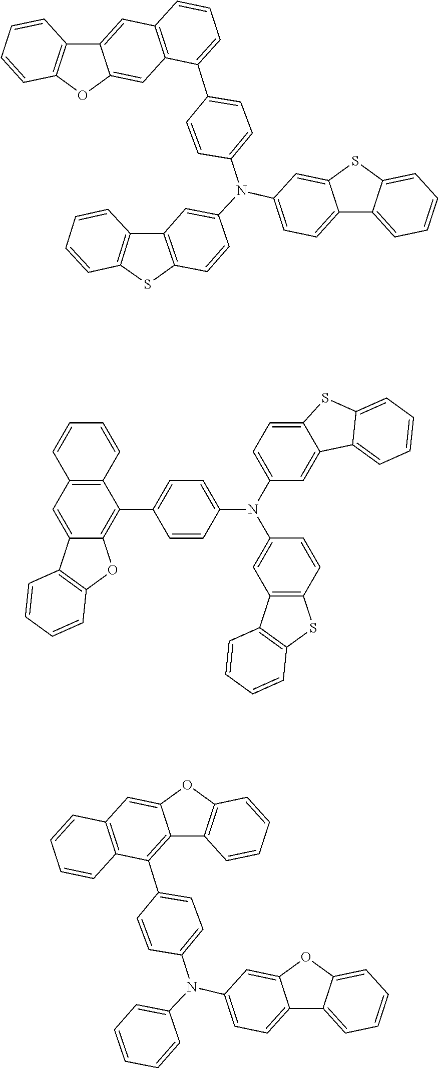 [SMILES: c1cc(-c2ccc(N(c3ccc4c(c3)sc3ccccc34)c3ccc4sc5ccccc5c4c3)cc2)c2cc3oc4ccccc4c3cc2c1 ; c1ccc2c(-c3ccc(N(c4ccc5sc6ccccc6c5c4)c4ccc5sc6ccccc6c5c4)cc3)c3oc4ccccc4c3cc2c1 ; c1ccc(N(c2ccc(-c3c4ccccc4cc4oc5ccccc5c34)cc2)c2ccc3c(c2)oc2ccccc23)cc1]

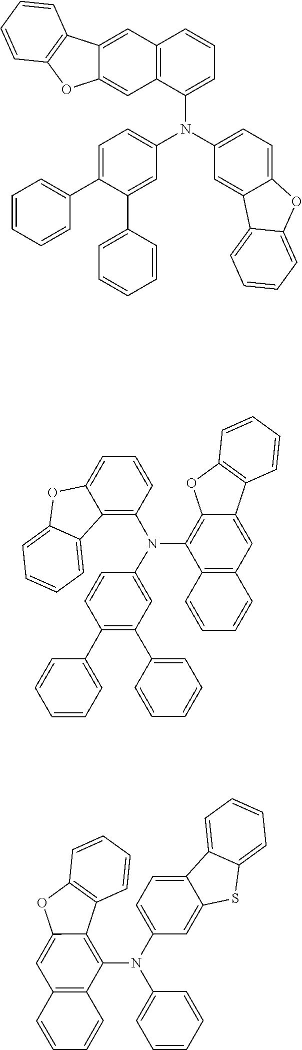 [SMILES: c1ccc(-c2ccc(N(c3ccc4oc5ccccc5c4c3)c3cccc4cc5c(cc34)oc3ccccc35)cc2-c2ccccc2)cc1 ; c1ccc(-c2ccc(N(c3c4ccccc4cc4c3oc3ccccc34)c3cccc4oc5ccccc5c34)cc2-c2ccccc2)cc1 ; c1ccc(N(c2ccc3c(c2)sc2ccccc23)c2c3ccccc3cc3oc4ccccc4c23)cc1]

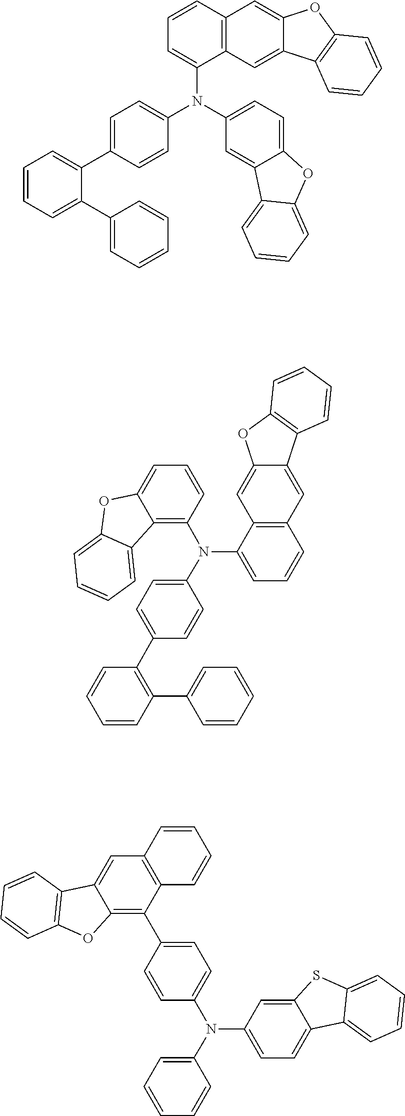 [SMILES: c1ccc(-c2ccccc2-c2ccc(N(c3ccc4oc5ccccc5c4c3)c3cccc4cc5oc6ccccc6c5cc34)cc2)cc1 ; c1ccc(-c2ccccc2-c2ccc(N(c3cccc4cc5c(cc34)oc3ccccc35)c3cccc4oc5ccccc5c34)cc2)cc1 ; c1ccc(N(c2ccc(-c3c4ccccc4cc4c3oc3ccccc34)cc2)c2ccc3c(c2)sc2ccccc23)cc1]

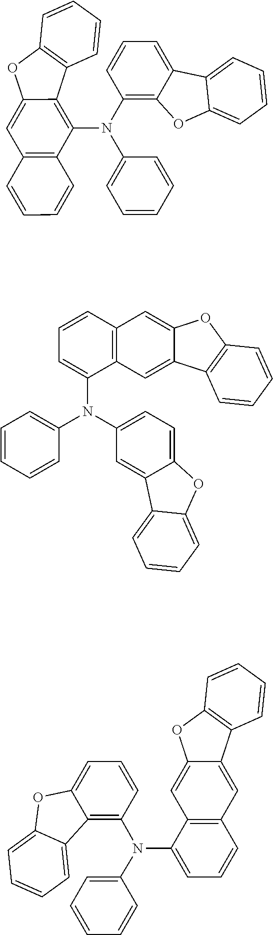 [SMILES: c1ccc(N(c2cccc3c2oc2ccccc23)c2c3ccccc3cc3oc4ccccc4c23)cc1 ; c1ccc(N(c2ccc3oc4ccccc4c3c2)c2cccc3cc4oc5ccccc5c4cc23)cc1 ; c1ccc(N(c2cccc3cc4c(cc23)oc2ccccc24)c2cccc3oc4ccccc4c23)cc1]

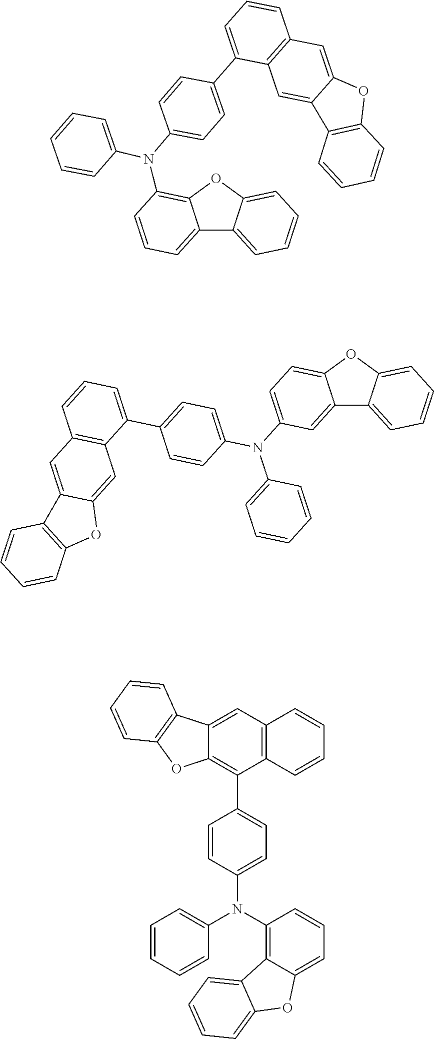 [SMILES: c1ccc(N(c2ccc(-c3cccc4cc5oc6ccccc6c5cc34)cc2)c2cccc3c2oc2ccccc23)cc1 ; c1ccc(N(c2ccc(-c3cccc4cc5c(cc34)oc3ccccc35)cc2)c2ccc3oc4ccccc4c3c2)cc1 ; c1ccc(N(c2ccc(-c3c4ccccc4cc4c3oc3ccccc34)cc2)c2cccc3oc4ccccc4c23)cc1]

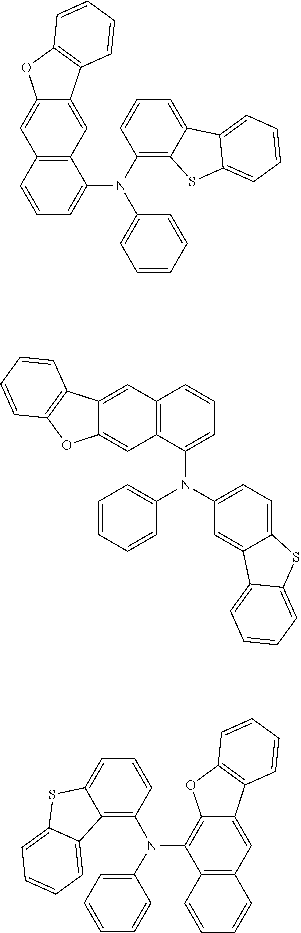 [SMILES: c1ccc(N(c2cccc3cc4oc5ccccc5c4cc23)c2cccc3c2sc2ccccc23)cc1 ; c1ccc(N(c2ccc3sc4ccccc4c3c2)c2cccc3cc4c(cc23)oc2ccccc24)cc1 ; c1ccc(N(c2c3ccccc3cc3c2oc2ccccc23)c2cccc3sc4ccccc4c23)cc1]

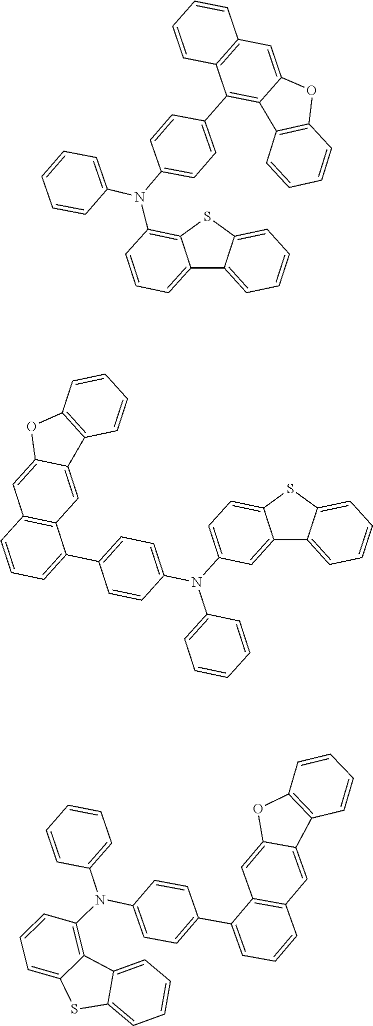 [SMILES: c1ccc(N(c2ccc(-c3c4ccccc4cc4oc5ccccc5c34)cc2)c2cccc3c2sc2ccccc23)cc1 ; c1ccc(N(c2ccc(-c3cccc4cc5oc6ccccc6c5cc34)cc2)c2ccc3sc4ccccc4c3c2)cc1 ; c1ccc(N(c2ccc(-c3cccc4cc5c(cc34)oc3ccccc35)cc2)c2cccc3sc4ccccc4c23)cc1]

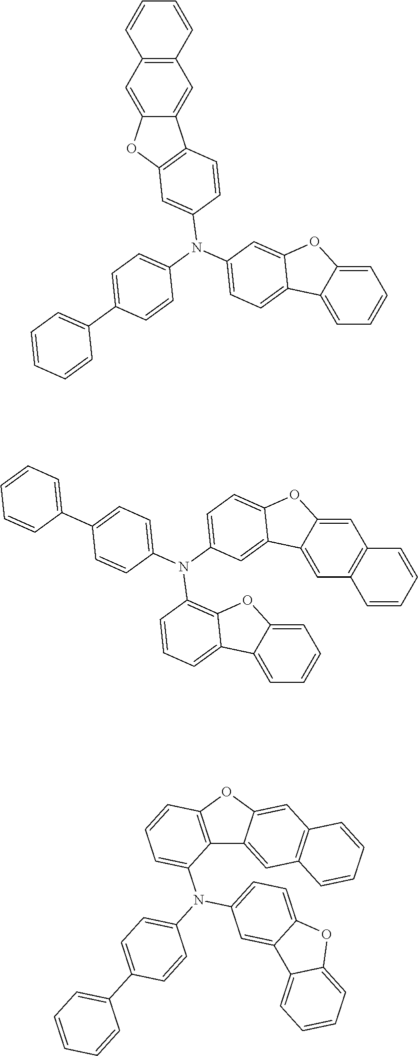 [SMILES: c1ccc(-c2ccc(N(c3ccc4c(c3)oc3ccccc34)c3ccc4c(c3)oc3cc5ccccc5cc34)cc2)cc1 ; c1ccc(-c2ccc(N(c3ccc4oc5cc6ccccc6cc5c4c3)c3cccc4c3oc3ccccc34)cc2)cc1 ; c1ccc(-c2ccc(N(c3ccc4oc5ccccc5c4c3)c3cccc4oc5cc6ccccc6cc5c34)cc2)cc1]

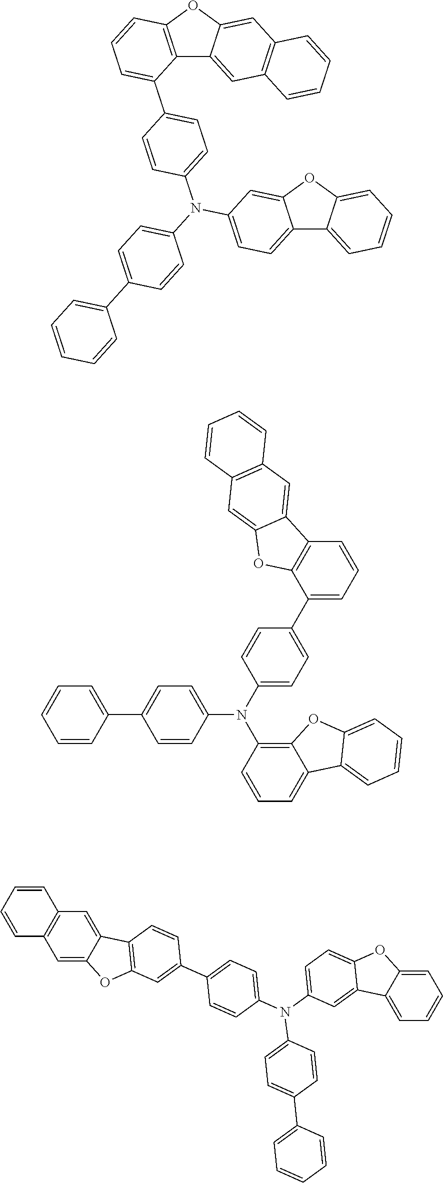 [SMILES: c1ccc(-c2ccc(N(c3ccc(-c4cccc5oc6cc7ccccc7cc6c45)cc3)c3ccc4c(c3)oc3ccccc34)cc2)cc1 ; c1ccc(-c2ccc(N(c3ccc(-c4cccc5c4oc4cc6ccccc6cc45)cc3)c3cccc4c3oc3ccccc34)cc2)cc1 ; c1ccc(-c2ccc(N(c3ccc(-c4ccc5c(c4)oc4cc6ccccc6cc45)cc3)c3ccc4oc5ccccc5c4c3)cc2)cc1]

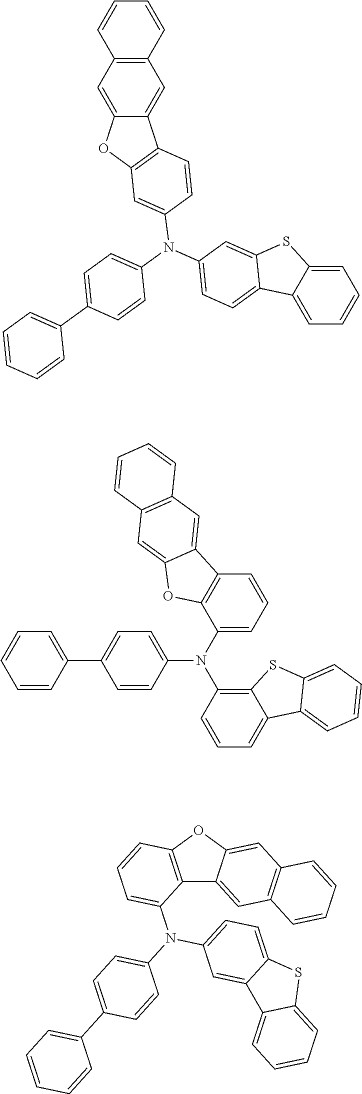 [SMILES: c1ccc(-c2ccc(N(c3ccc4c(c3)oc3cc5ccccc5cc34)c3ccc4c(c3)sc3ccccc34)cc2)cc1 ; c1ccc(-c2ccc(N(c3cccc4c3oc3cc5ccccc5cc34)c3cccc4c3sc3ccccc34)cc2)cc1 ; c1ccc(-c2ccc(N(c3ccc4sc5ccccc5c4c3)c3cccc4oc5cc6ccccc6cc5c34)cc2)cc1]

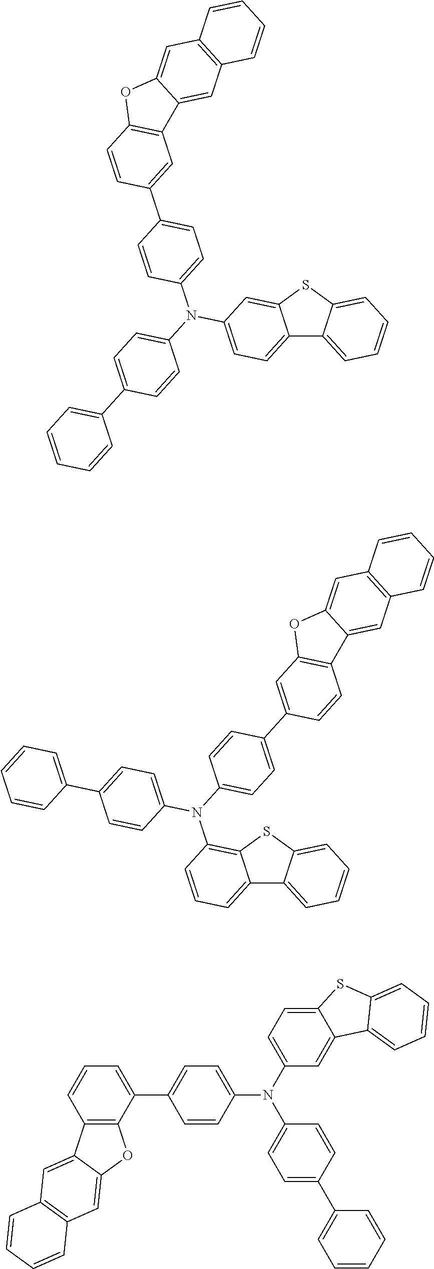 [SMILES: c1ccc(-c2ccc(N(c3ccc(-c4ccc5oc6cc7ccccc7cc6c5c4)cc3)c3ccc4c(c3)sc3ccccc34)cc2)cc1 ; c1ccc(-c2ccc(N(c3ccc(-c4ccc5c(c4)oc4cc6ccccc6cc45)cc3)c3cccc4c3sc3ccccc34)cc2)cc1 ; c1ccc(-c2ccc(N(c3ccc(-c4cccc5c4oc4cc6ccccc6cc45)cc3)c3ccc4sc5ccccc5c4c3)cc2)cc1]

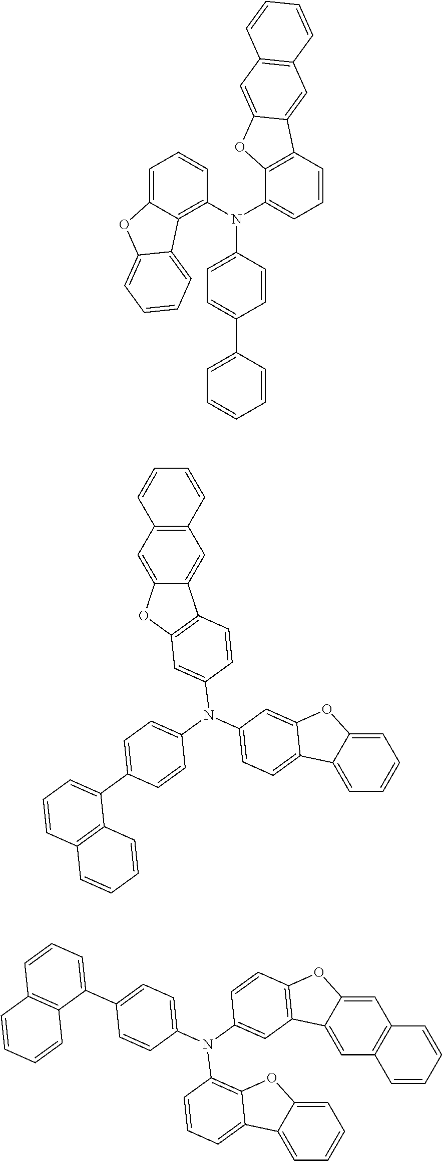 [SMILES: c1ccc(-c2ccc(N(c3cccc4c3oc3cc5ccccc5cc34)c3cccc4oc5ccccc5c34)cc2)cc1 ; c1ccc2cc3c(cc2c1)oc1cc(N(c2ccc(-c4cccc5ccccc45)cc2)c2ccc4c(c2)oc2ccccc24)ccc13 ; c1ccc2cc3c(cc2c1)oc1ccc(N(c2ccc(-c4cccc5ccccc45)cc2)c2cccc4c2oc2ccccc24)cc13]

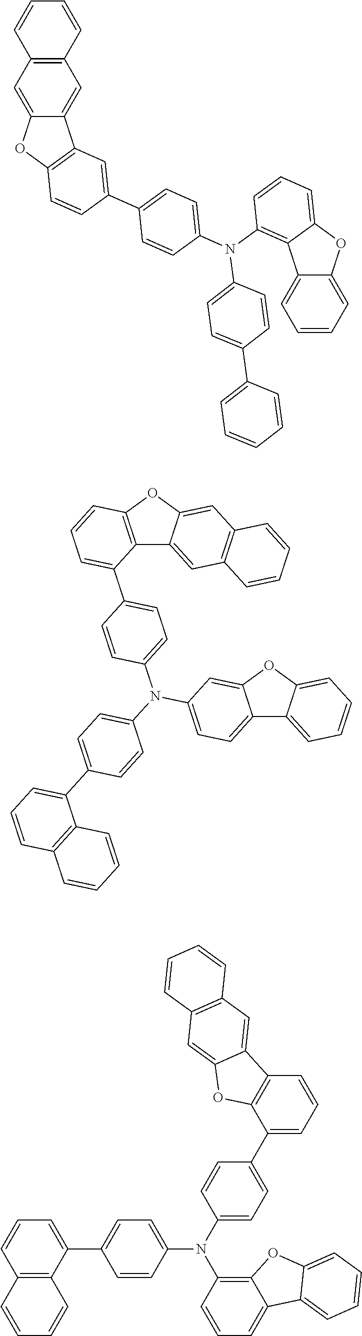 [SMILES: c1ccc(-c2ccc(N(c3ccc(-c4ccc5oc6cc7ccccc7cc6c5c4)cc3)c3cccc4oc5ccccc5c34)cc2)cc1 ; c1ccc2cc3c(cc2c1)oc1cccc(-c2ccc(N(c4ccc(-c5cccc6ccccc56)cc4)c4ccc5c(c4)oc4ccccc45)cc2)c13 ; c1ccc2cc3c(cc2c1)oc1c(-c2ccc(N(c4ccc(-c5cccc6ccccc56)cc4)c4cccc5c4oc4ccccc45)cc2)cccc13]

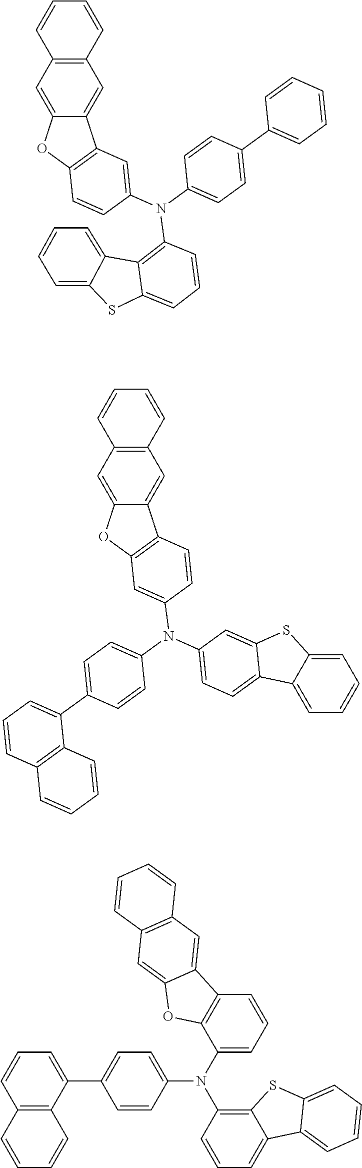 [SMILES: c1ccc(-c2ccc(N(c3ccc4oc5cc6ccccc6cc5c4c3)c3cccc4sc5ccccc5c34)cc2)cc1 ; c1ccc2cc3c(cc2c1)oc1cc(N(c2ccc(-c4cccc5ccccc45)cc2)c2ccc4c(c2)sc2ccccc24)ccc13 ; c1ccc2cc3c(cc2c1)oc1c(N(c2ccc(-c4cccc5ccccc45)cc2)c2cccc4c2sc2ccccc24)cccc13]

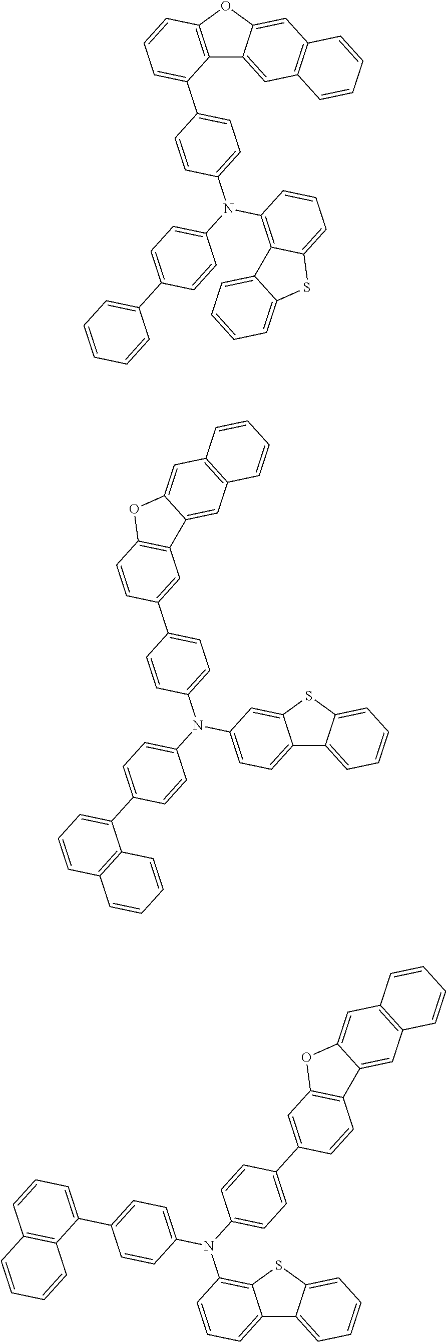 [SMILES: c1ccc(-c2ccc(N(c3ccc(-c4cccc5oc6cc7ccccc7cc6c45)cc3)c3cccc4sc5ccccc5c34)cc2)cc1 ; c1ccc2cc3c(cc2c1)oc1ccc(-c2ccc(N(c4ccc(-c5cccc6ccccc56)cc4)c4ccc5c(c4)sc4ccccc45)cc2)cc13 ; c1ccc2cc3c(cc2c1)oc1cc(-c2ccc(N(c4ccc(-c5cccc6ccccc56)cc4)c4cccc5c4sc4ccccc45)cc2)ccc13]

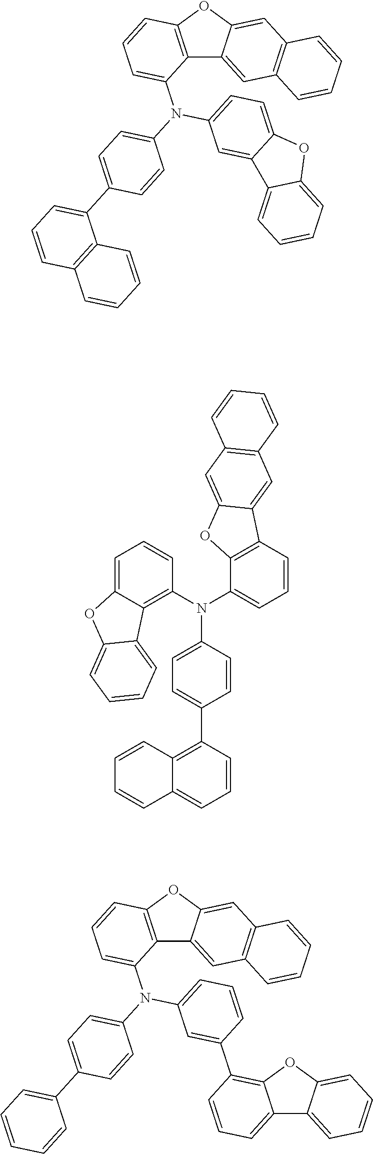 [SMILES: c1ccc2cc3c(cc2c1)oc1cccc(N(c2ccc(-c4cccc5ccccc45)cc2)c2ccc4oc5ccccc5c4c2)c13 ; c1ccc2cc3c(cc2c1)oc1c(N(c2ccc(-c4cccc5ccccc45)cc2)c2cccc4oc5ccccc5c24)cccc13 ; c1ccc(-c2ccc(N(c3cccc(-c4cccc5c4oc4ccccc45)c3)c3cccc4oc5cc6ccccc6cc5c34)cc2)cc1]

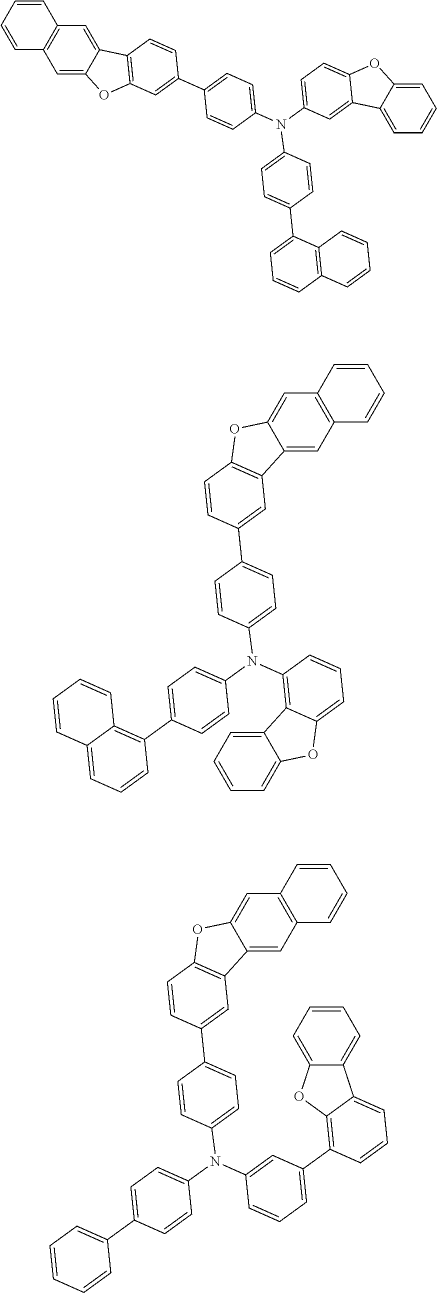 [SMILES: c1ccc2cc3c(cc2c1)oc1cc(-c2ccc(N(c4ccc(-c5cccc6ccccc56)cc4)c4ccc5oc6ccccc6c5c4)cc2)ccc13 ; c1ccc2cc3c(cc2c1)oc1ccc(-c2ccc(N(c4ccc(-c5cccc6ccccc56)cc4)c4cccc5oc6ccccc6c45)cc2)cc13 ; c1ccc(-c2ccc(N(c3ccc(-c4ccc5oc6cc7ccccc7cc6c5c4)cc3)c3cccc(-c4cccc5c4oc4ccccc45)c3)cc2)cc1]

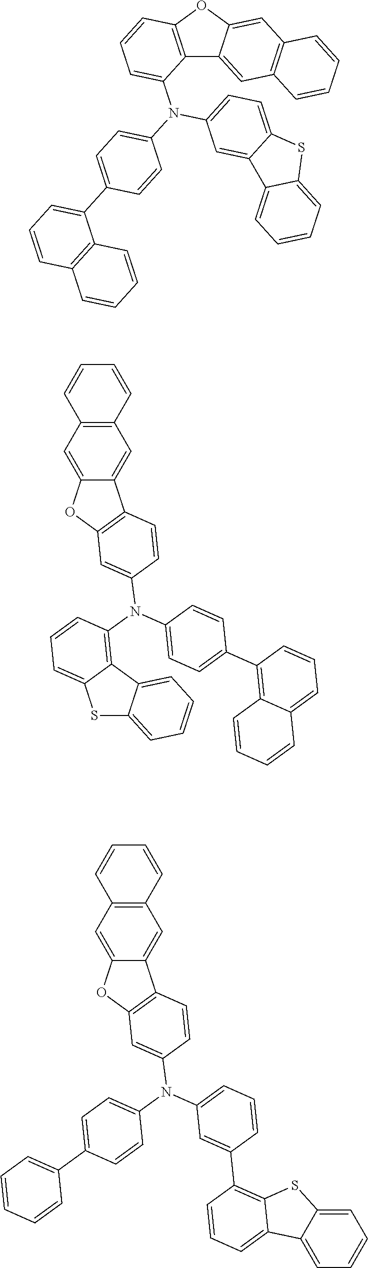 [SMILES: c1ccc2cc3c(cc2c1)oc1cccc(N(c2ccc(-c4cccc5ccccc45)cc2)c2ccc4sc5ccccc5c4c2)c13 ; c1ccc2cc3c(cc2c1)oc1cc(N(c2ccc(-c4cccc5ccccc45)cc2)c2cccc4sc5ccccc5c24)ccc13 ; c1ccc(-c2ccc(N(c3cccc(-c4cccc5c4sc4ccccc45)c3)c3ccc4c(c3)oc3cc5ccccc5cc34)cc2)cc1]

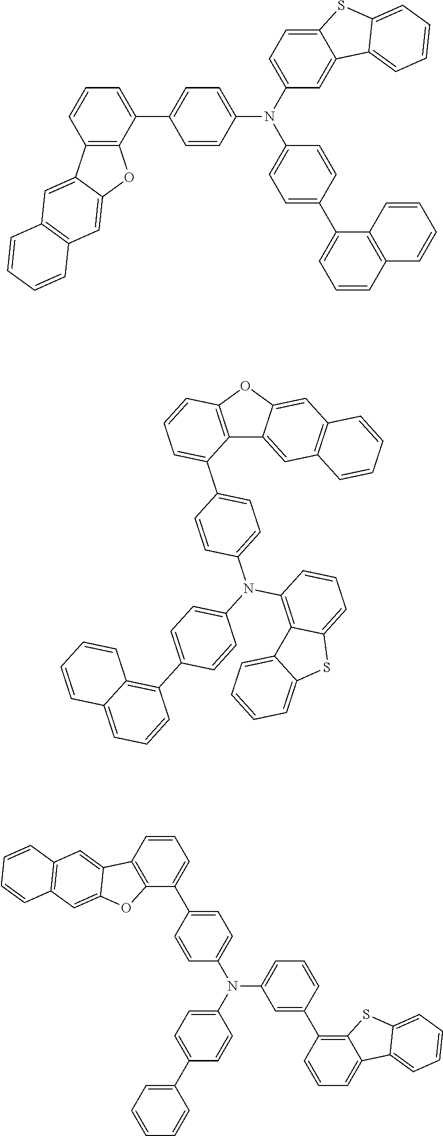 [SMILES: c1ccc2cc3c(cc2c1)oc1c(-c2ccc(N(c4ccc(-c5cccc6ccccc56)cc4)c4ccc5sc6ccccc6c5c4)cc2)cccc13 ; c1ccc2cc3c(cc2c1)oc1cccc(-c2ccc(N(c4ccc(-c5cccc6ccccc56)cc4)c4cccc5sc6ccccc6c45)cc2)c13 ; c1ccc(-c2ccc(N(c3ccc(-c4cccc5c4oc4cc6ccccc6cc45)cc3)c3cccc(-c4cccc5c4sc4ccccc45)c3)cc2)cc1]

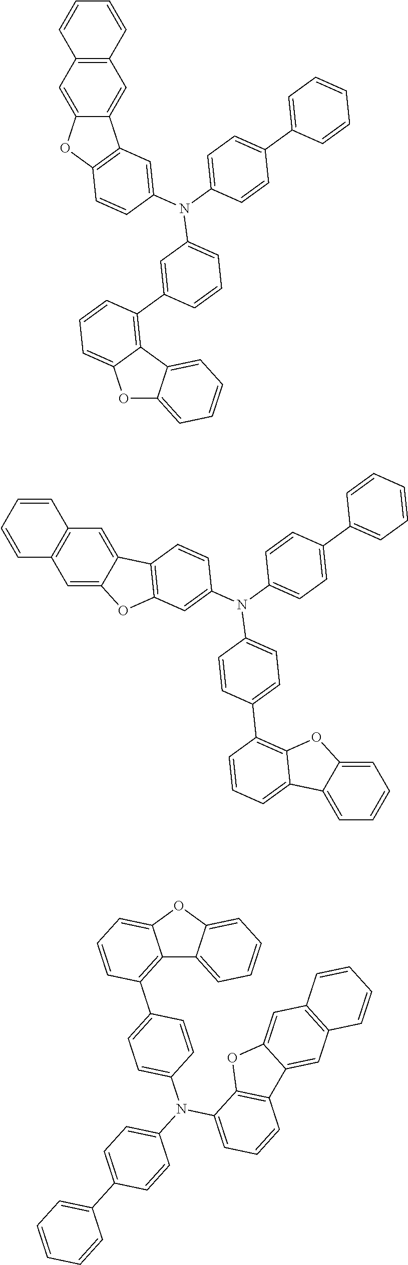 [SMILES: c1ccc(-c2ccc(N(c3cccc(-c4cccc5oc6ccccc6c45)c3)c3ccc4oc5cc6ccccc6cc5c4c3)cc2)cc1 ; c1ccc(-c2ccc(N(c3ccc(-c4cccc5c4oc4ccccc45)cc3)c3ccc4c(c3)oc3cc5ccccc5cc34)cc2)cc1 ; c1ccc(-c2ccc(N(c3ccc(-c4cccc5oc6ccccc6c45)cc3)c3cccc4c3oc3cc5ccccc5cc34)cc2)cc1]

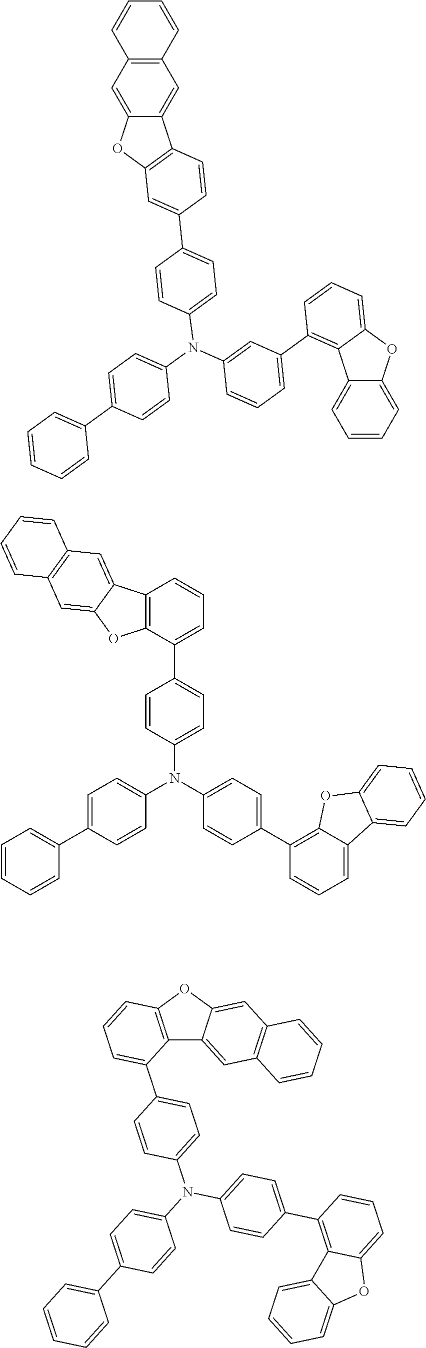

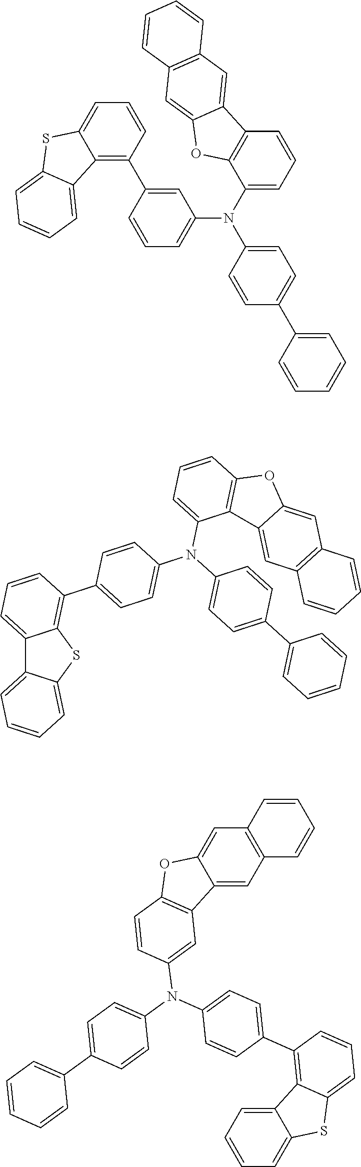 [SMILES: c1ccc(-c2ccc(N(c3cccc(-c4cccc5sc6ccccc6c45)c3)c3cccc4c3oc3cc5ccccc5cc34)cc2)cc1 ; c1ccc(-c2ccc(N(c3ccc(-c4cccc5c4sc4ccccc45)cc3)c3cccc4oc5cc6ccccc6cc5c34)cc2)cc1 ; c1ccc(-c2ccc(N(c3ccc(-c4cccc5sc6ccccc6c45)cc3)c3ccc4oc5cc6ccccc6cc5c4c3)cc2)cc1]

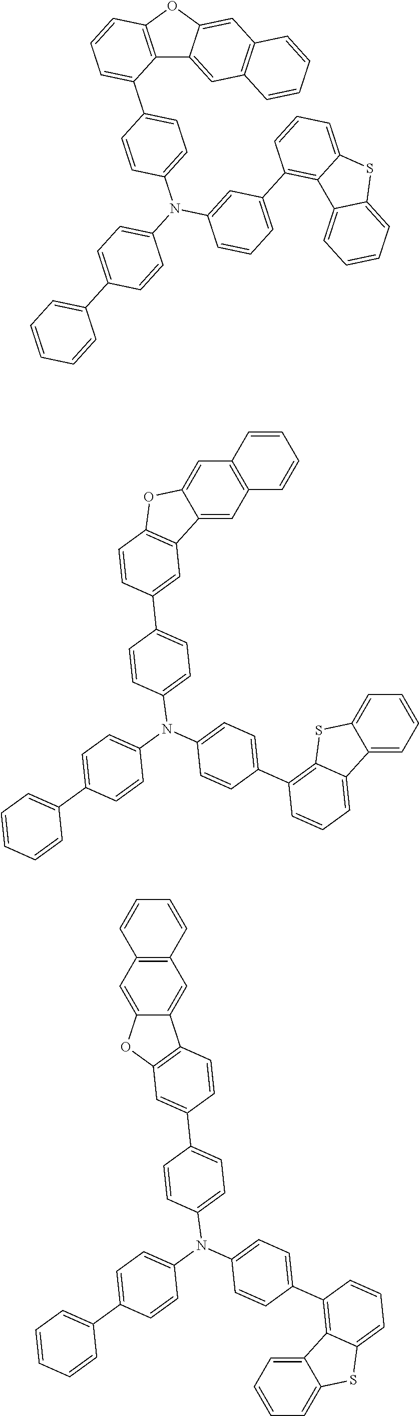 [SMILES: c1ccc(-c2ccc(N(c3ccc(-c4cccc5oc6cc7ccccc7cc6c45)cc3)c3cccc(-c4cccc5sc6ccccc6c45)c3)cc2)cc1 ; c1ccc(-c2ccc(N(c3ccc(-c4ccc5oc6cc7ccccc7cc6c5c4)cc3)c3ccc(-c4cccc5c4sc4ccccc45)cc3)cc2)cc1 ; c1ccc(-c2ccc(N(c3ccc(-c4ccc5c(c4)oc4cc6ccccc6cc45)cc3)c3ccc(-c4cccc5sc6ccccc6c45)cc3)cc2)cc1]

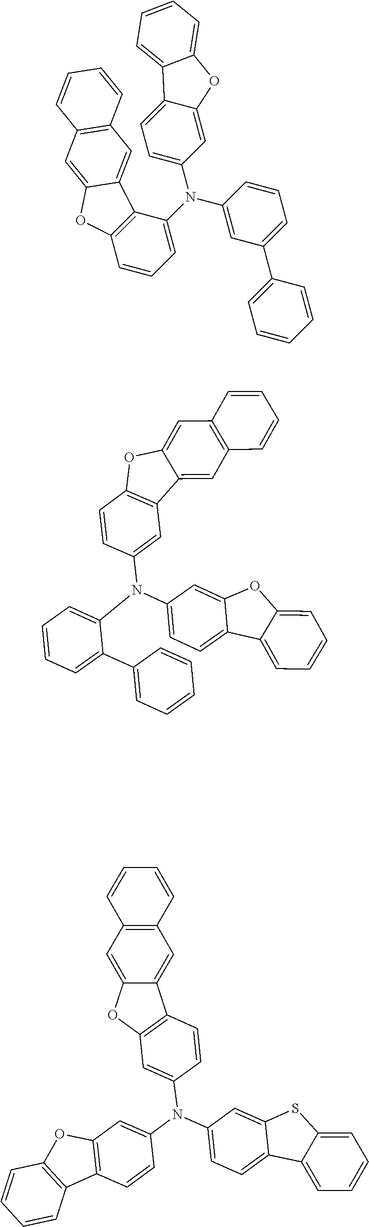 [SMILES: c1ccc(-c2cccc(N(c3ccc4c(c3)oc3ccccc34)c3cccc4oc5cc6ccccc6cc5c34)c2)cc1 ; c1ccc(-c2ccccc2N(c2ccc3c(c2)oc2ccccc23)c2ccc3oc4cc5ccccc5cc4c3c2)cc1 ; c1ccc2cc3c(cc2c1)oc1cc(N(c2ccc4c(c2)oc2ccccc24)c2ccc4c(c2)sc2ccccc24)ccc13]

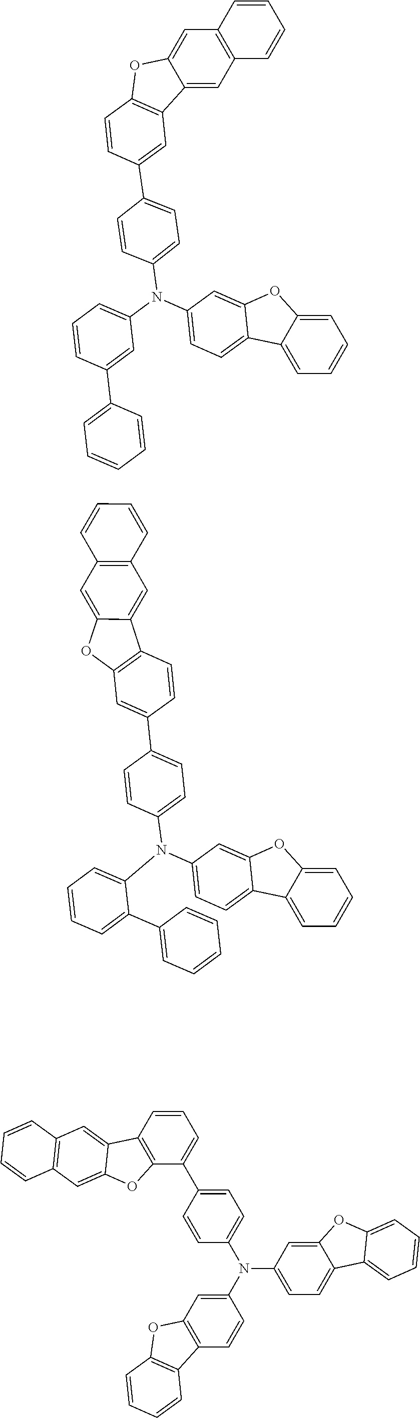

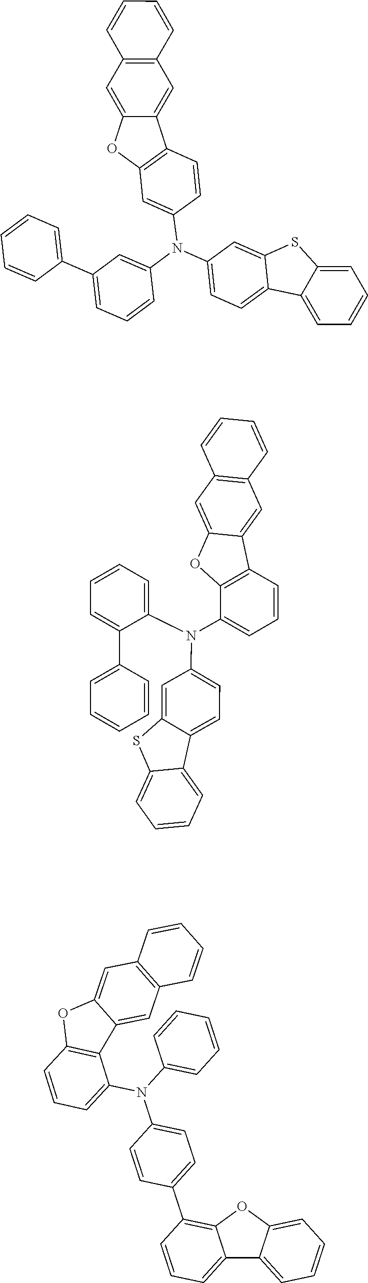 [SMILES: c1ccc(-c2cccc(N(c3ccc4c(c3)oc3cc5ccccc5cc34)c3ccc4c(c3)sc3ccccc34)c2)cc1 ; c1ccc(-c2ccccc2N(c2ccc3c(c2)sc2ccccc23)c2cccc3c2oc2cc4ccccc4cc23)cc1 ; c1ccc(N(c2ccc(-c3cccc4c3oc3ccccc34)cc2)c2cccc3oc4cc5ccccc5cc4c23)cc1]

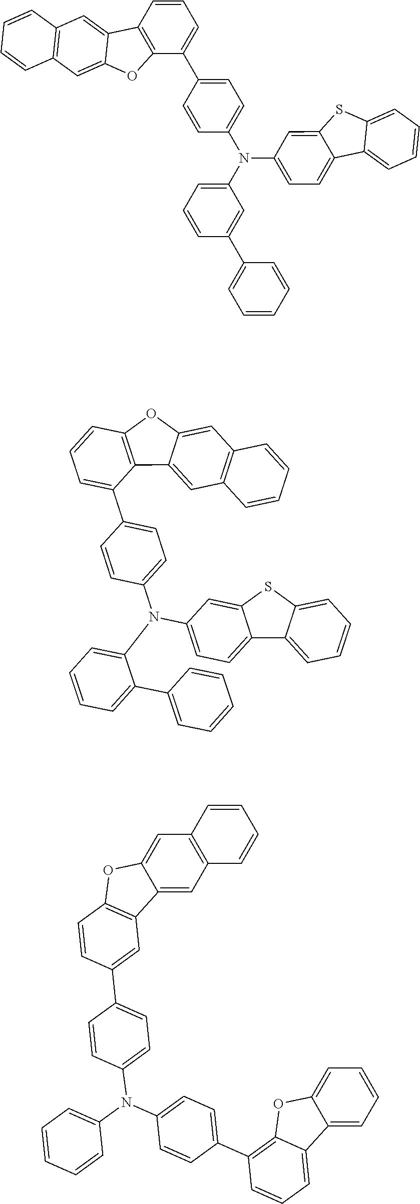 [SMILES: c1ccc(-c2cccc(N(c3ccc(-c4cccc5c4oc4cc6ccccc6cc45)cc3)c3ccc4c(c3)sc3ccccc34)c2)cc1 ; c1ccc(-c2ccccc2N(c2ccc(-c3cccc4oc5cc6ccccc6cc5c34)cc2)c2ccc3c(c2)sc2ccccc23)cc1 ; c1ccc(N(c2ccc(-c3ccc4oc5cc6ccccc6cc5c4c3)cc2)c2ccc(-c3cccc4c3oc3ccccc34)cc2)cc1]

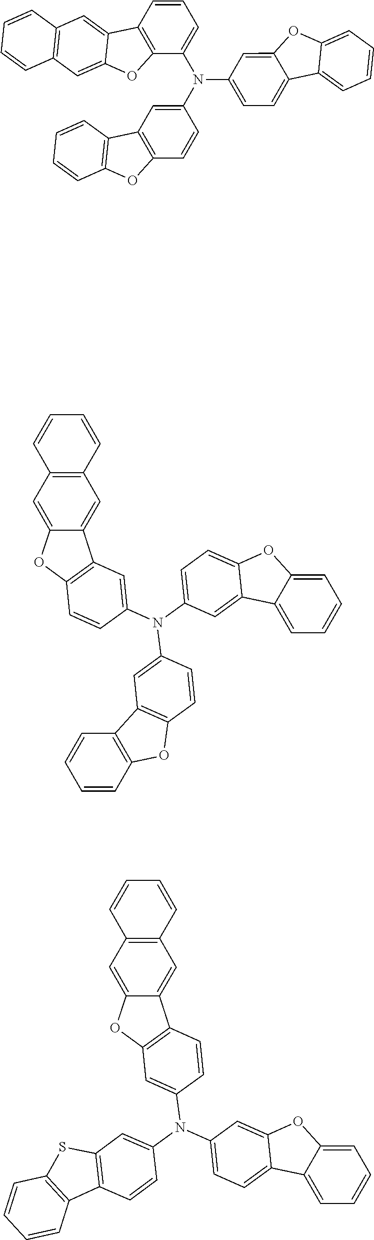 [SMILES: c1ccc2cc3c(cc2c1)oc1c(N(c2ccc4c(c2)oc2ccccc24)c2ccc4oc5ccccc5c4c2)cccc13 ; c1ccc2cc3c(cc2c1)oc1ccc(N(c2ccc4oc5ccccc5c4c2)c2ccc4oc5ccccc5c4c2)cc13 ; c1ccc2cc3c(cc2c1)oc1cc(N(c2ccc4c(c2)oc2ccccc24)c2ccc4c(c2)sc2ccccc24)ccc13]

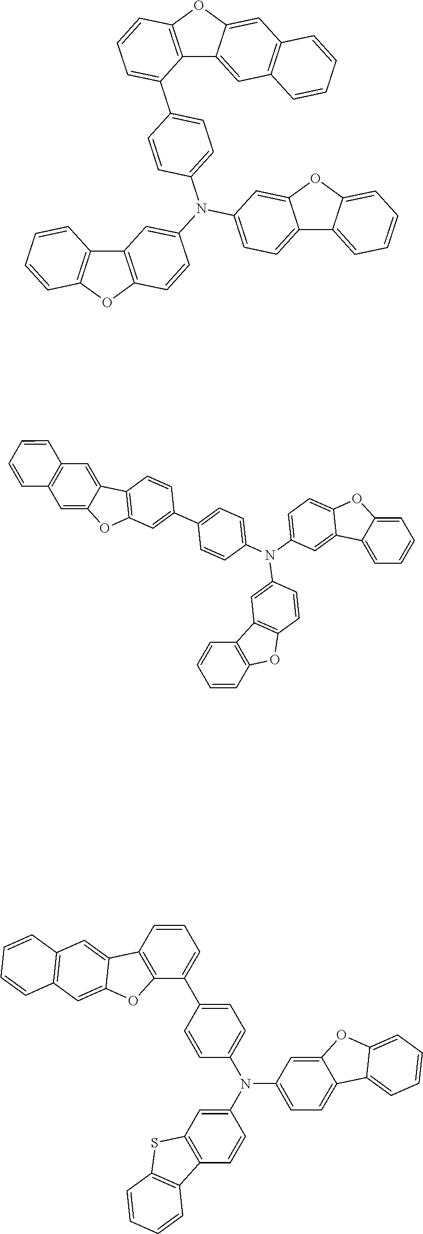 [SMILES: c1ccc2cc3c(cc2c1)oc1cccc(-c2ccc(N(c4ccc5c(c4)oc4ccccc45)c4ccc5oc6ccccc6c5c4)cc2)c13 ; c1ccc2cc3c(cc2c1)oc1cc(-c2ccc(N(c4ccc5oc6ccccc6c5c4)c4ccc5oc6ccccc6c5c4)cc2)ccc13 ; c1ccc2cc3c(cc2c1)oc1c(-c2ccc(N(c4ccc5c(c4)oc4ccccc45)c4ccc5c(c4)sc4ccccc45)cc2)cccc13]

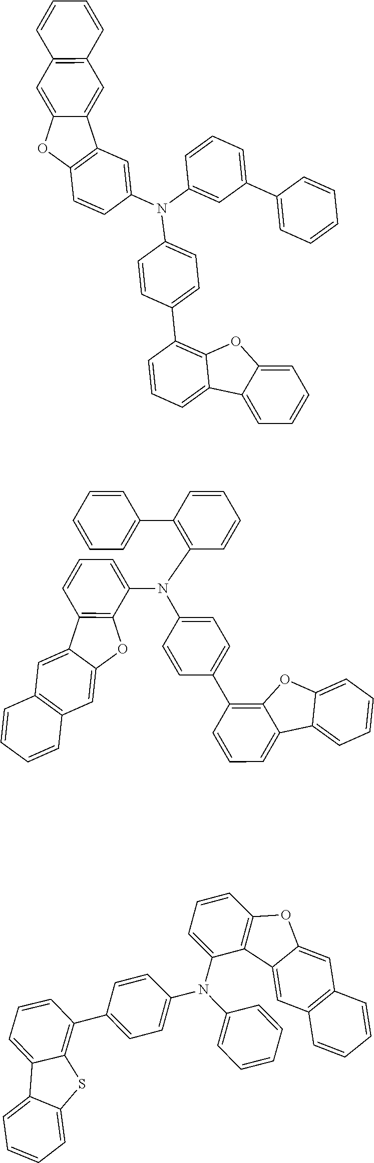 [SMILES: c1ccc(-c2cccc(N(c3ccc(-c4cccc5c4oc4ccccc45)cc3)c3ccc4oc5cc6ccccc6cc5c4c3)c2)cc1 ; c1ccc(-c2ccccc2N(c2ccc(-c3cccc4c3oc3ccccc34)cc2)c2cccc3c2oc2cc4ccccc4cc23)cc1 ; c1ccc(N(c2ccc(-c3cccc4c3sc3ccccc34)cc2)c2cccc3oc4cc5ccccc5cc4c23)cc1]

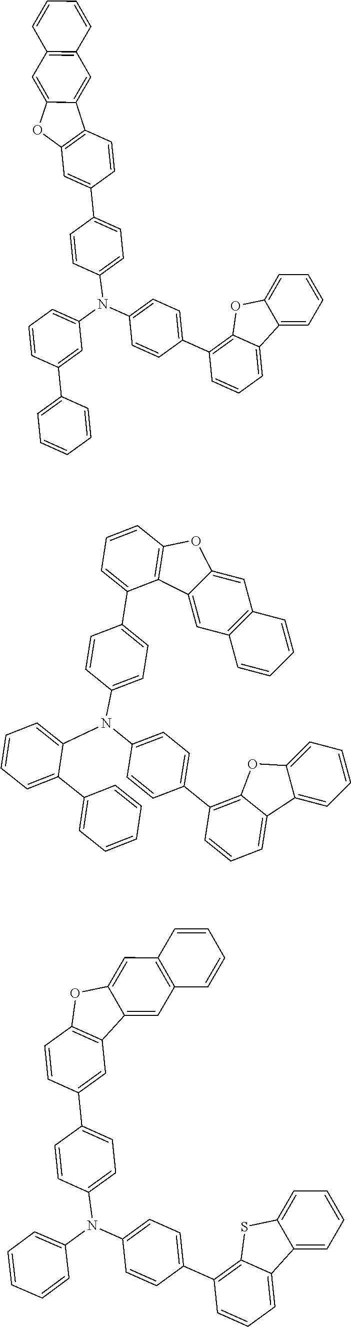 [SMILES: c1ccc(-c2cccc(N(c3ccc(-c4ccc5c(c4)oc4cc6ccccc6cc45)cc3)c3ccc(-c4cccc5c4oc4ccccc45)cc3)c2)cc1 ; c1ccc(-c2ccccc2N(c2ccc(-c3cccc4c3oc3ccccc34)cc2)c2ccc(-c3cccc4oc5cc6ccccc6cc5c34)cc2)cc1 ; c1ccc(N(c2ccc(-c3ccc4oc5cc6ccccc6cc5c4c3)cc2)c2ccc(-c3cccc4c3sc3ccccc34)cc2)cc1]

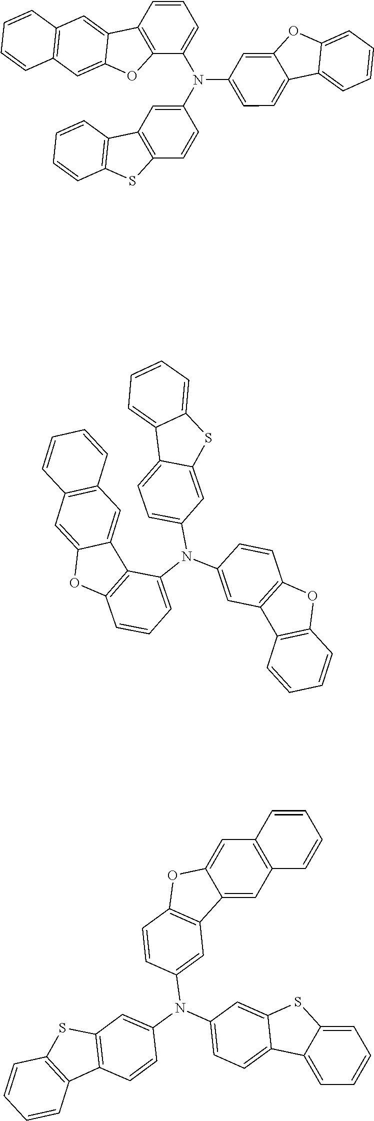 [SMILES: c1ccc2cc3c(cc2c1)oc1c(N(c2ccc4c(c2)oc2ccccc24)c2ccc4sc5ccccc5c4c2)cccc13 ; c1ccc2cc3c(cc2c1)oc1cccc(N(c2ccc4c(c2)sc2ccccc24)c2ccc4oc5ccccc5c4c2)c13 ; c1ccc2cc3c(cc2c1)oc1ccc(N(c2ccc4c(c2)sc2ccccc24)c2ccc4c(c2)sc2ccccc24)cc13]

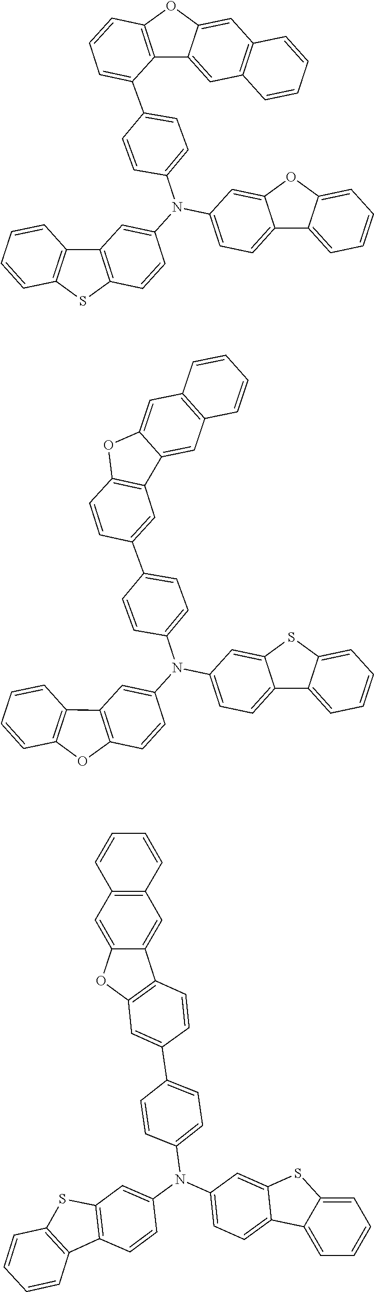 [SMILES: c1ccc2cc3c(cc2c1)oc1cccc(-c2ccc(N(c4ccc5c(c4)oc4ccccc45)c4ccc5sc6ccccc6c5c4)cc2)c13 ; c1ccc2cc3c(cc2c1)oc1ccc(-c2ccc(N(c4ccc5c(c4)sc4ccccc45)c4ccc5oc6ccccc6c5c4)cc2)cc13 ; c1ccc2cc3c(cc2c1)oc1cc(-c2ccc(N(c4ccc5c(c4)sc4ccccc45)c4ccc5c(c4)sc4ccccc45)cc2)ccc13]

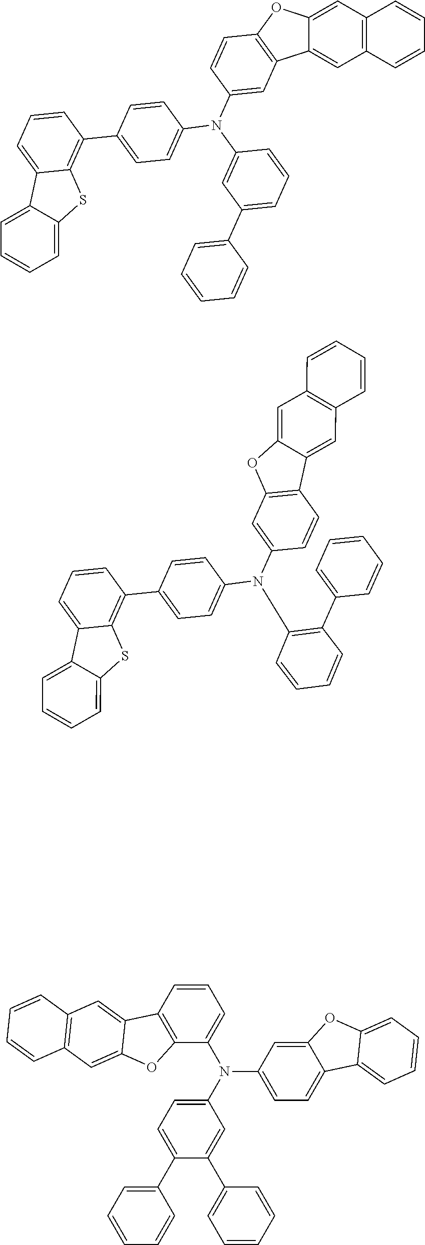 [SMILES: c1ccc(-c2cccc(N(c3ccc(-c4cccc5c4sc4ccccc45)cc3)c3ccc4oc5cc6ccccc6cc5c4c3)c2)cc1 ; c1ccc(-c2ccccc2N(c2ccc(-c3cccc4c3sc3ccccc34)cc2)c2ccc3c(c2)oc2cc4ccccc4cc23)cc1 ; c1ccc(-c2ccc(N(c3ccc4c(c3)oc3ccccc34)c3cccc4c3oc3cc5ccccc5cc34)cc2-c2ccccc2)cc1]

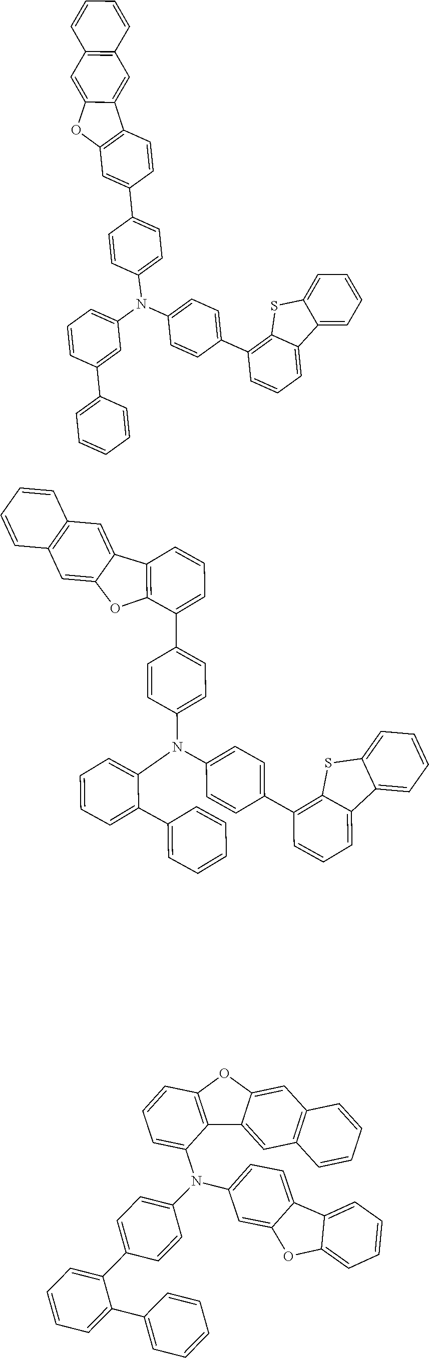 [SMILES: c1ccc(-c2cccc(N(c3ccc(-c4ccc5c(c4)oc4cc6ccccc6cc45)cc3)c3ccc(-c4cccc5c4sc4ccccc45)cc3)c2)cc1 ; c1ccc(-c2ccccc2N(c2ccc(-c3cccc4c3oc3cc5ccccc5cc34)cc2)c2ccc(-c3cccc4c3sc3ccccc34)cc2)cc1 ; c1ccc(-c2ccccc2-c2ccc(N(c3ccc4c(c3)oc3ccccc34)c3cccc4oc5cc6ccccc6cc5c34)cc2)cc1]

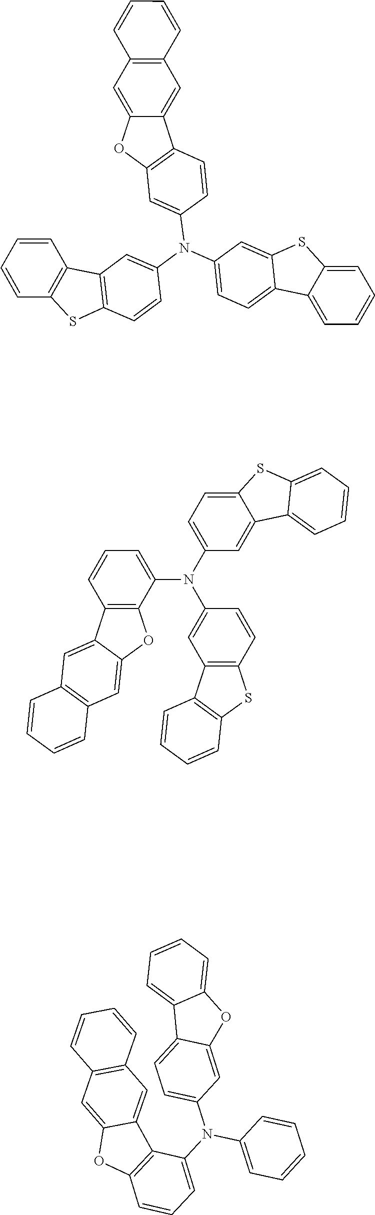 [SMILES: c1ccc2cc3c(cc2c1)oc1cc(N(c2ccc4c(c2)sc2ccccc24)c2ccc4sc5ccccc5c4c2)ccc13 ; c1ccc2cc3c(cc2c1)oc1c(N(c2ccc4sc5ccccc5c4c2)c2ccc4sc5ccccc5c4c2)cccc13 ; c1ccc(N(c2ccc3c(c2)oc2ccccc23)c2cccc3oc4cc5ccccc5cc4c23)cc1]

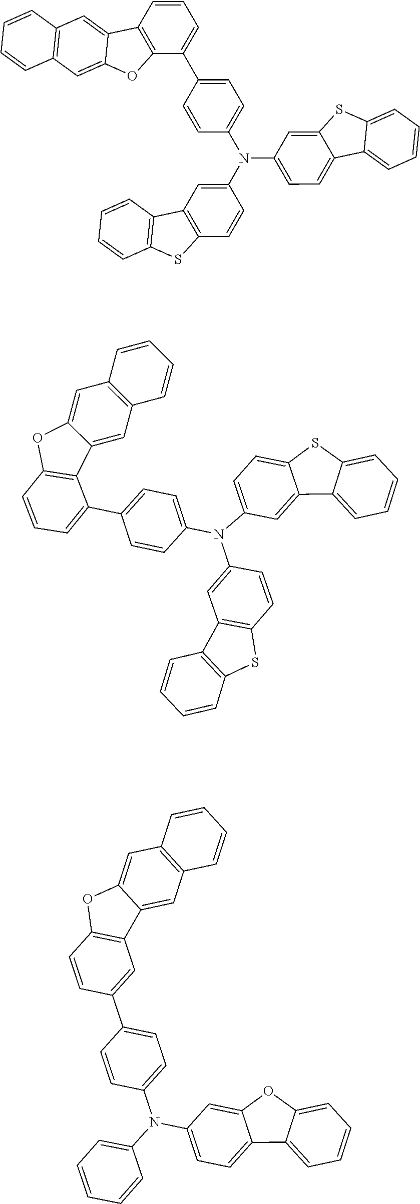 [SMILES: c1ccc2cc3c(cc2c1)oc1c(-c2ccc(N(c4ccc5c(c4)sc4ccccc45)c4ccc5sc6ccccc6c5c4)cc2)cccc13 ; c1ccc2cc3c(cc2c1)oc1cccc(-c2ccc(N(c4ccc5sc6ccccc6c5c4)c4ccc5sc6ccccc6c5c4)cc2)c13 ; c1ccc(N(c2ccc(-c3ccc4oc5cc6ccccc6cc5c4c3)cc2)c2ccc3c(c2)oc2ccccc23)cc1]

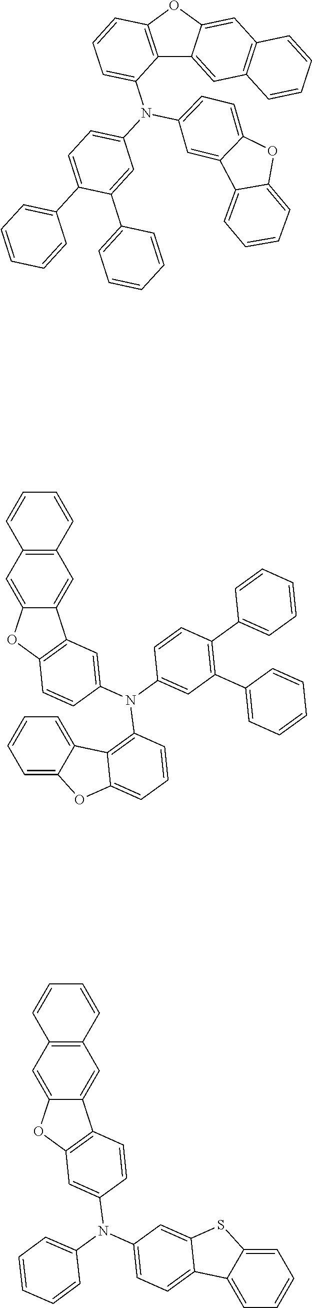 [SMILES: c1ccc(-c2ccc(N(c3ccc4oc5ccccc5c4c3)c3cccc4oc5cc6ccccc6cc5c34)cc2-c2ccccc2)cc1 ; c1ccc(-c2ccc(N(c3ccc4oc5cc6ccccc6cc5c4c3)c3cccc4oc5ccccc5c34)cc2-c2ccccc2)cc1 ; c1ccc(N(c2ccc3c(c2)oc2cc4ccccc4cc23)c2ccc3c(c2)sc2ccccc23)cc1]

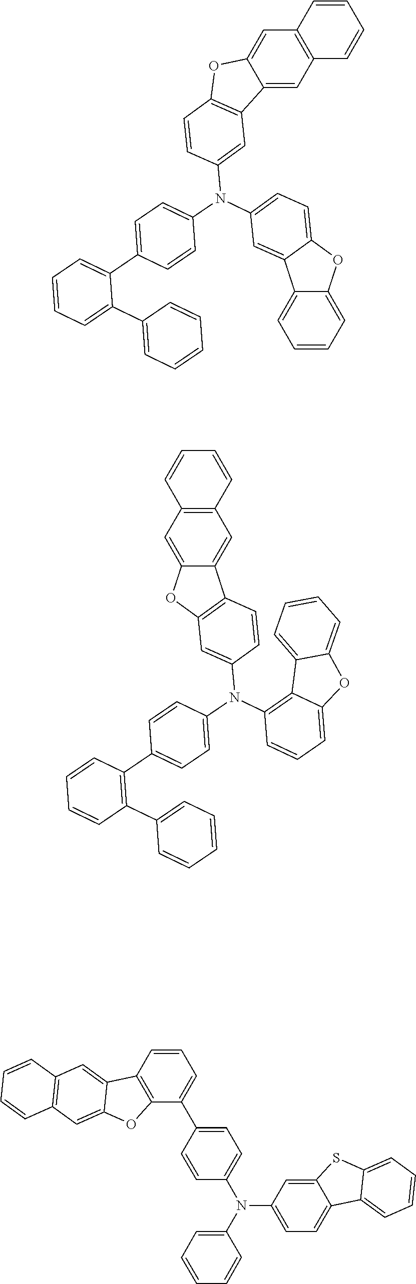 [SMILES: c1ccc(-c2ccccc2-c2ccc(N(c3ccc4oc5ccccc5c4c3)c3ccc4oc5cc6ccccc6cc5c4c3)cc2)cc1 ; c1ccc(-c2ccccc2-c2ccc(N(c3ccc4c(c3)oc3cc5ccccc5cc34)c3cccc4oc5ccccc5c34)cc2)cc1 ; c1ccc(N(c2ccc(-c3cccc4c3oc3cc5ccccc5cc34)cc2)c2ccc3c(c2)sc2ccccc23)cc1]

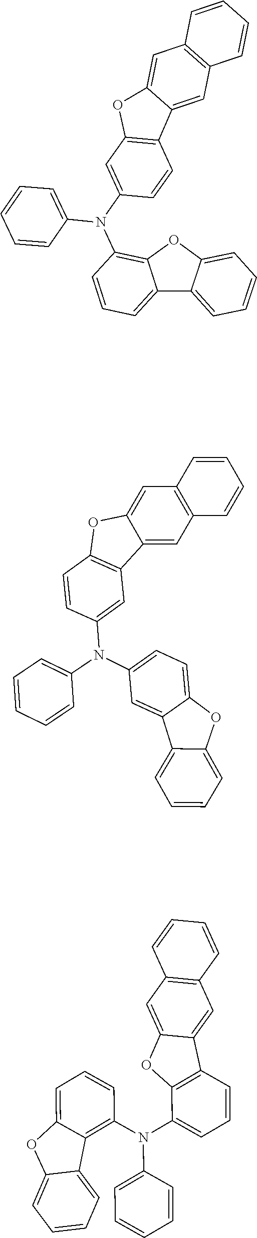 [SMILES: c1ccc(N(c2ccc3c(c2)oc2cc4ccccc4cc23)c2cccc3c2oc2ccccc23)cc1 ; c1ccc(N(c2ccc3oc4ccccc4c3c2)c2ccc3oc4cc5ccccc5cc4c3c2)cc1 ; c1ccc(N(c2cccc3c2oc2cc4ccccc4cc23)c2cccc3oc4ccccc4c23)cc1]

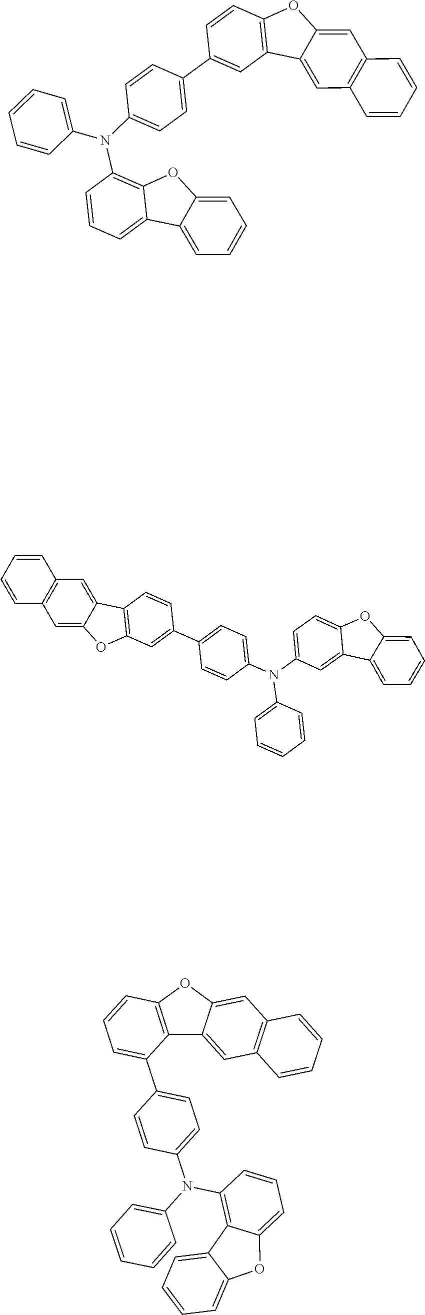 [SMILES: c1ccc(N(c2ccc(-c3ccc4oc5cc6ccccc6cc5c4c3)cc2)c2cccc3c2oc2ccccc23)cc1 ; c1ccc(N(c2ccc(-c3ccc4c(c3)oc3cc5ccccc5cc34)cc2)c2ccc3oc4ccccc4c3c2)cc1 ; c1ccc(N(c2ccc(-c3cccc4oc5cc6ccccc6cc5c34)cc2)c2cccc3oc4ccccc4c23)cc1]

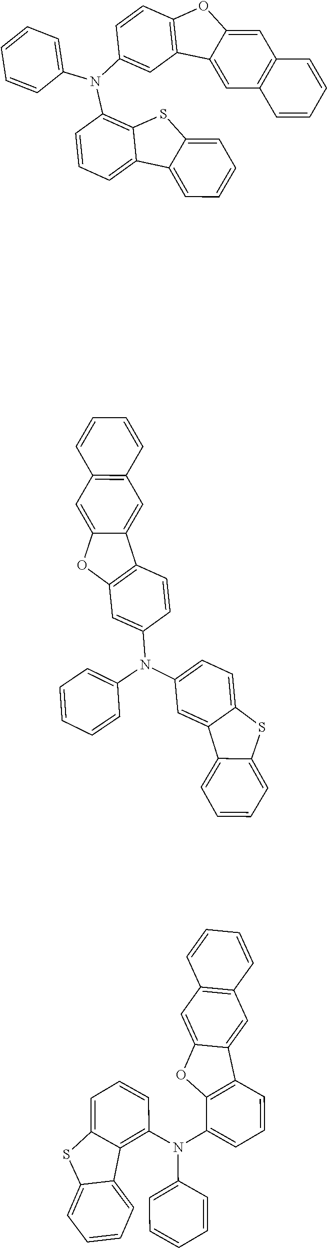 [SMILES: c1ccc(N(c2ccc3oc4cc5ccccc5cc4c3c2)c2cccc3c2sc2ccccc23)cc1 ; c1ccc(N(c2ccc3c(c2)oc2cc4ccccc4cc23)c2ccc3sc4ccccc4c3c2)cc1 ; c1ccc(N(c2cccc3c2oc2cc4ccccc4cc23)c2cccc3sc4ccccc4c23)cc1]

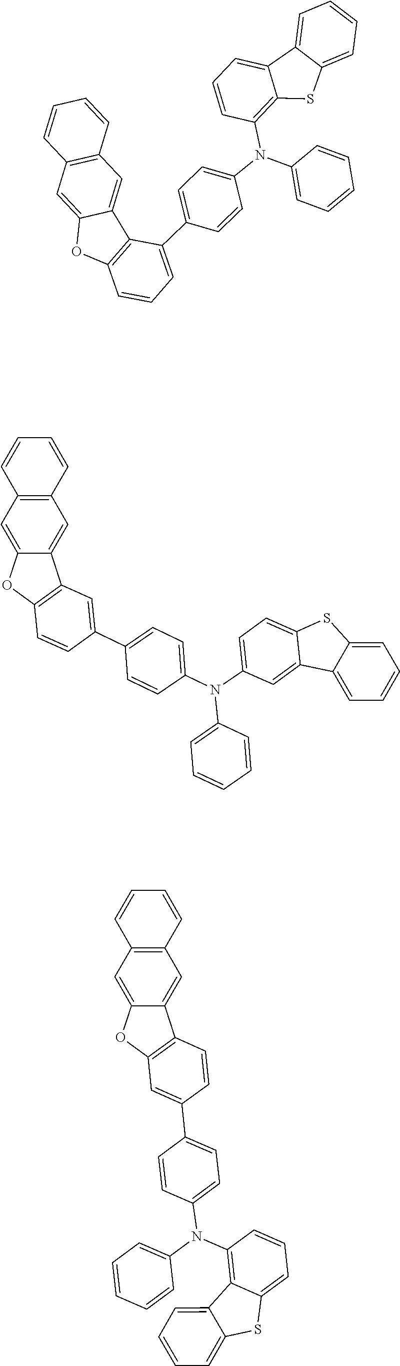 [SMILES: c1ccc(N(c2ccc(-c3cccc4oc5cc6ccccc6cc5c34)cc2)c2cccc3c2sc2ccccc23)cc1 ; c1ccc(N(c2ccc(-c3ccc4oc5cc6ccccc6cc5c4c3)cc2)c2ccc3sc4ccccc4c3c2)cc1 ; c1ccc(N(c2ccc(-c3ccc4c(c3)oc3cc5ccccc5cc34)cc2)c2cccc3sc4ccccc4c23)cc1]

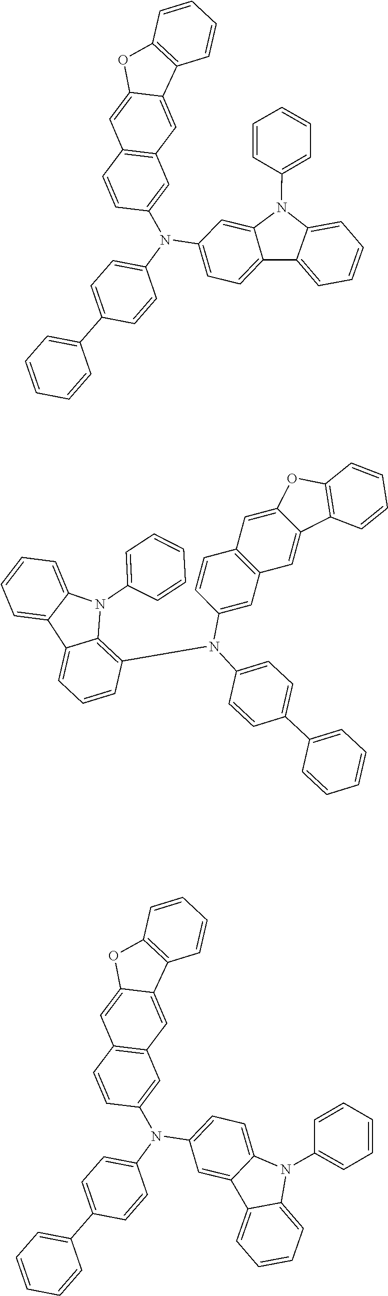 [SMILES: c1ccc(-c2ccc(N(c3ccc4cc5oc6ccccc6c5cc4c3)c3ccc4c5ccccc5n(-c5ccccc5)c4c3)cc2)cc1 ; c1ccc(-c2ccc(N(c3ccc4cc5oc6ccccc6c5cc4c3)c3cccc4c5ccccc5n(-c5ccccc5)c34)cc2)cc1 ; c1ccc(-c2ccc(N(c3ccc4cc5oc6ccccc6c5cc4c3)c3ccc4c(c3)c3ccccc3n4-c3ccccc3)cc2)cc1]

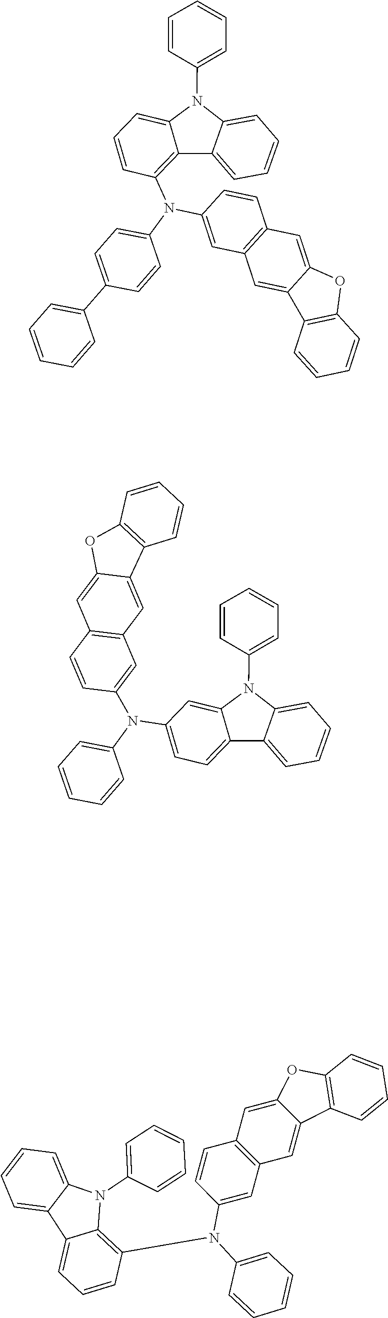 [SMILES: c1ccc(-c2ccc(N(c3ccc4cc5oc6ccccc6c5cc4c3)c3cccc4c3c3ccccc3n4-c3ccccc3)cc2)cc1 ; c1ccc(N(c2ccc3cc4oc5ccccc5c4cc3c2)c2ccc3c4ccccc4n(-c4ccccc4)c3c2)cc1 ; c1ccc(N(c2ccc3cc4oc5ccccc5c4cc3c2)c2cccc3c4ccccc4n(-c4ccccc4)c23)cc1]

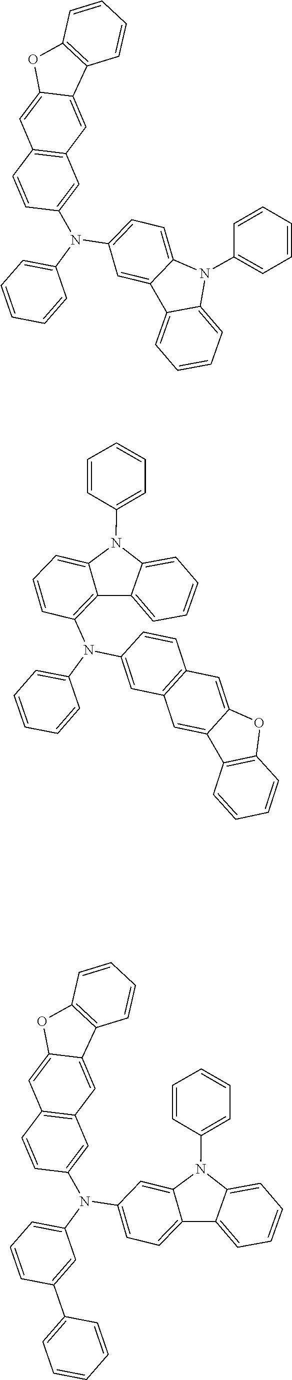 [SMILES: c1ccc(N(c2ccc3cc4oc5ccccc5c4cc3c2)c2ccc3c(c2)c2ccccc2n3-c2ccccc2)cc1 ; c1ccc(N(c2ccc3cc4oc5ccccc5c4cc3c2)c2cccc3c2c2ccccc2n3-c2ccccc2)cc1 ; c1ccc(-c2cccc(N(c3ccc4cc5oc6ccccc6c5cc4c3)c3ccc4c5ccccc5n(-c5ccccc5)c4c3)c2)cc1]

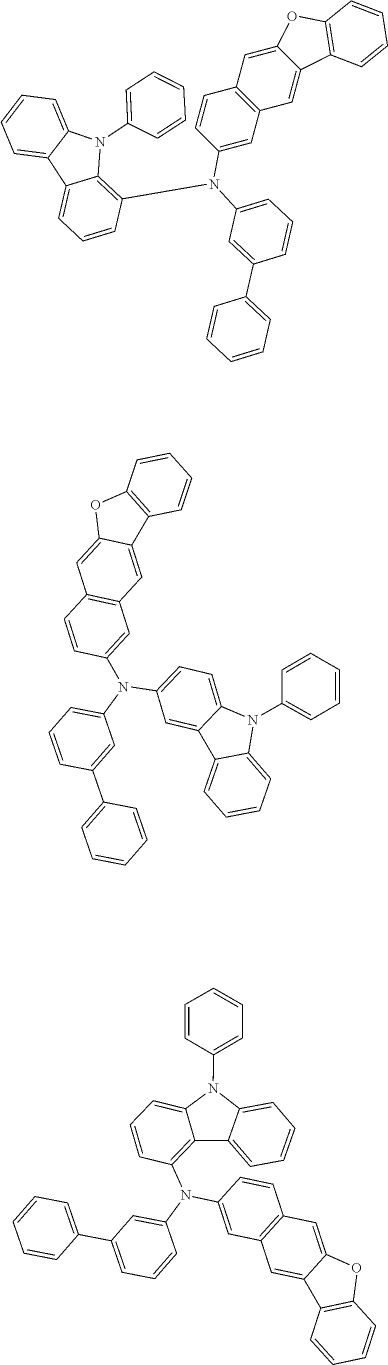 [SMILES: c1ccc(-c2cccc(N(c3ccc4cc5oc6ccccc6c5cc4c3)c3cccc4c5ccccc5n(-c5ccccc5)c34)c2)cc1 ; c1ccc(-c2cccc(N(c3ccc4cc5oc6ccccc6c5cc4c3)c3ccc4c(c3)c3ccccc3n4-c3ccccc3)c2)cc1 ; c1ccc(-c2cccc(N(c3ccc4cc5oc6ccccc6c5cc4c3)c3cccc4c3c3ccccc3n4-c3ccccc3)c2)cc1]

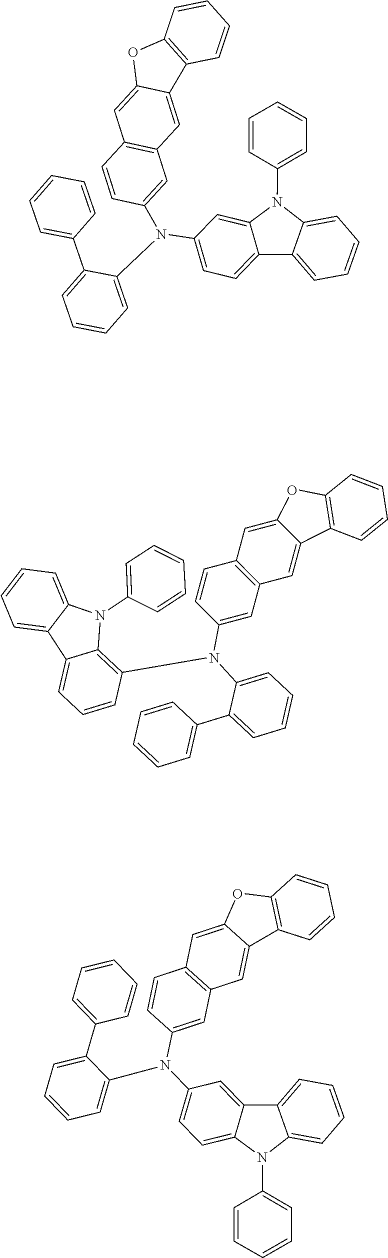 [SMILES: c1ccc(-c2ccccc2N(c2ccc3cc4oc5ccccc5c4cc3c2)c2ccc3c4ccccc4n(-c4ccccc4)c3c2)cc1 ; c1ccc(-c2ccccc2N(c2ccc3cc4oc5ccccc5c4cc3c2)c2cccc3c4ccccc4n(-c4ccccc4)c23)cc1 ; c1ccc(-c2ccccc2N(c2ccc3cc4oc5ccccc5c4cc3c2)c2ccc3c(c2)c2ccccc2n3-c2ccccc2)cc1]

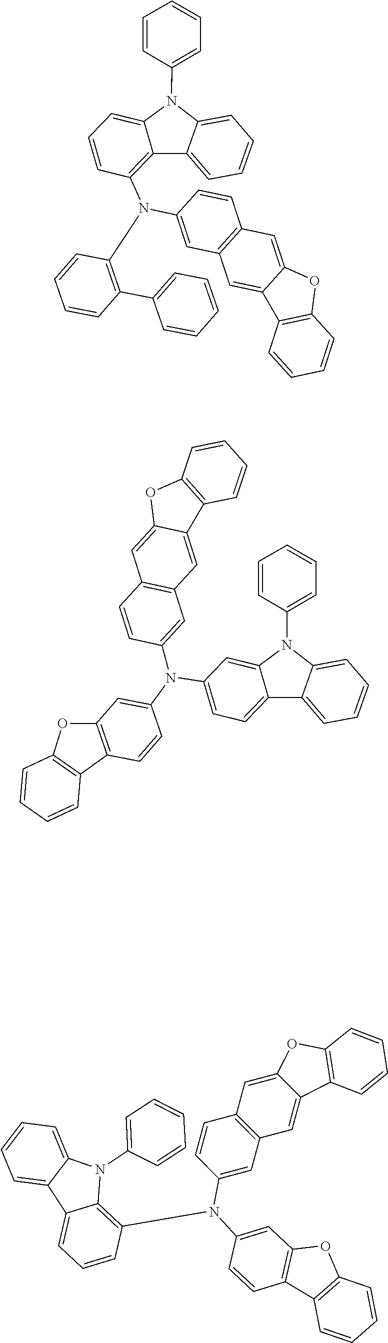 [SMILES: c1ccc(-c2ccccc2N(c2ccc3cc4oc5ccccc5c4cc3c2)c2cccc3c2c2ccccc2n3-c2ccccc2)cc1 ; c1ccc(-n2c3ccccc3c3ccc(N(c4ccc5cc6oc7ccccc7c6cc5c4)c4ccc5c(c4)oc4ccccc45)cc32)cc1 ; c1ccc(-n2c3ccccc3c3cccc(N(c4ccc5cc6oc7ccccc7c6cc5c4)c4ccc5c(c4)oc4ccccc45)c32)cc1]

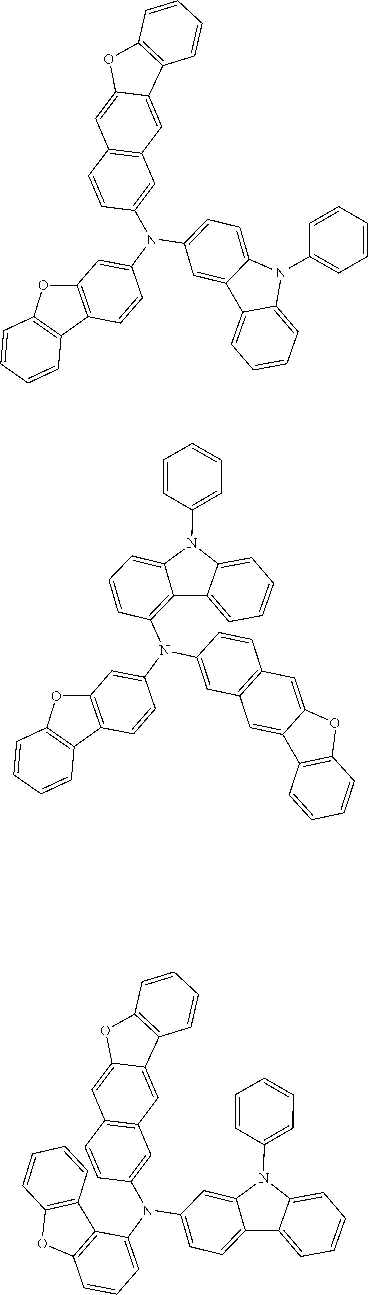 [SMILES: c1ccc(-n2c3ccccc3c3cc(N(c4ccc5cc6oc7ccccc7c6cc5c4)c4ccc5c(c4)oc4ccccc45)ccc32)cc1 ; c1ccc(-n2c3ccccc3c3c(N(c4ccc5cc6oc7ccccc7c6cc5c4)c4ccc5c(c4)oc4ccccc45)cccc32)cc1 ; c1ccc(-n2c3ccccc3c3ccc(N(c4ccc5cc6oc7ccccc7c6cc5c4)c4cccc5oc6ccccc6c45)cc32)cc1]

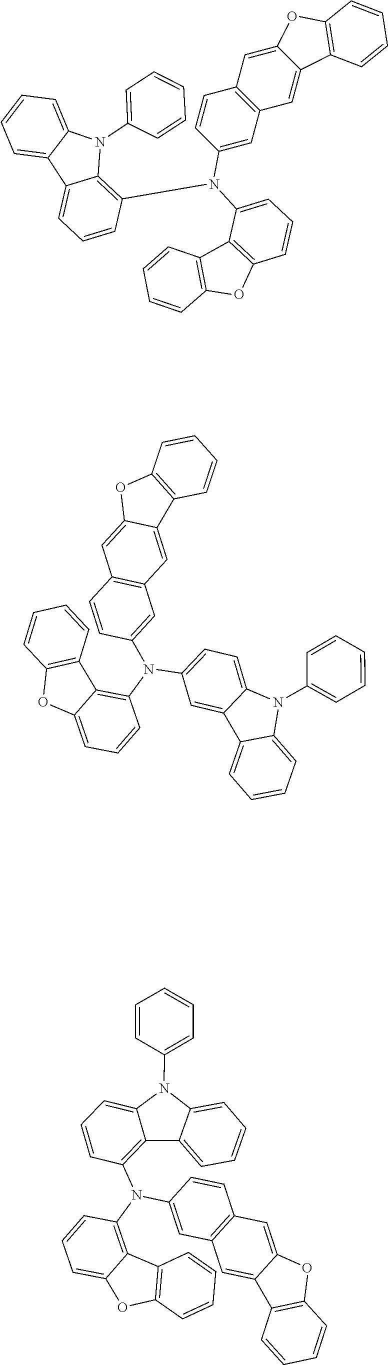 [SMILES: c1ccc(-n2c3ccccc3c3cccc(N(c4ccc5cc6oc7ccccc7c6cc5c4)c4cccc5oc6ccccc6c45)c32)cc1 ; c1ccc(-n2c3ccccc3c3cc(N(c4ccc5cc6oc7ccccc7c6cc5c4)c4cccc5oc6ccccc6c45)ccc32)cc1 ; c1ccc(-n2c3ccccc3c3c(N(c4ccc5cc6oc7ccccc7c6cc5c4)c4cccc5oc6ccccc6c45)cccc32)cc1]

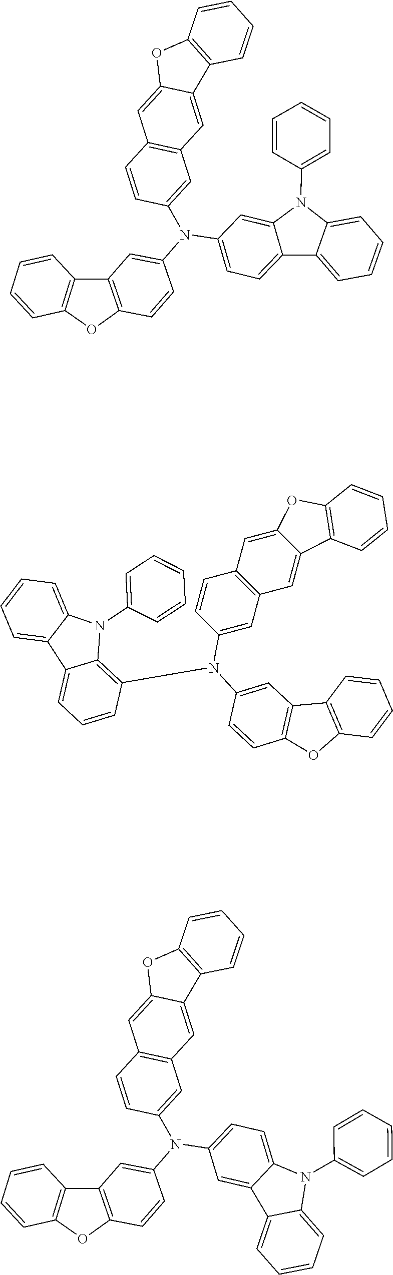 [SMILES: c1ccc(-n2c3ccccc3c3ccc(N(c4ccc5cc6oc7ccccc7c6cc5c4)c4ccc5oc6ccccc6c5c4)cc32)cc1 ; c1ccc(-n2c3ccccc3c3cccc(N(c4ccc5cc6oc7ccccc7c6cc5c4)c4ccc5oc6ccccc6c5c4)c32)cc1 ; c1ccc(-n2c3ccccc3c3cc(N(c4ccc5cc6oc7ccccc7c6cc5c4)c4ccc5oc6ccccc6c5c4)ccc32)cc1]

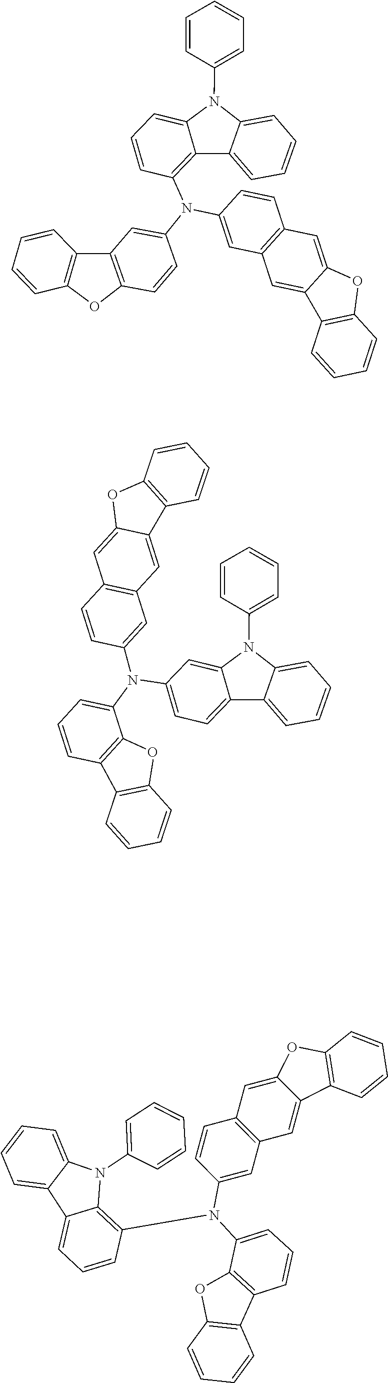 [SMILES: c1ccc(-n2c3ccccc3c3c(N(c4ccc5cc6oc7ccccc7c6cc5c4)c4ccc5oc6ccccc6c5c4)cccc32)cc1 ; c1ccc(-n2c3ccccc3c3ccc(N(c4ccc5cc6oc7ccccc7c6cc5c4)c4cccc5c4oc4ccccc45)cc32)cc1 ; c1ccc(-n2c3ccccc3c3cccc(N(c4ccc5cc6oc7ccccc7c6cc5c4)c4cccc5c4oc4ccccc45)c32)cc1]

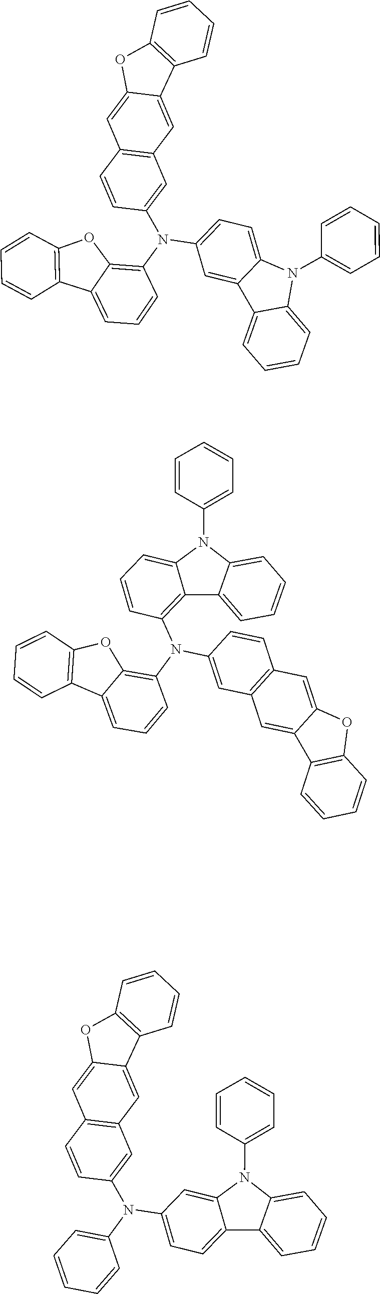 [SMILES: c1ccc(-n2c3ccccc3c3cc(N(c4ccc5cc6oc7ccccc7c6cc5c4)c4cccc5c4oc4ccccc45)ccc32)cc1 ; c1ccc(-n2c3ccccc3c3c(N(c4ccc5cc6oc7ccccc7c6cc5c4)c4cccc5c4oc4ccccc45)cccc32)cc1 ; c1ccc(N(c2ccc3cc4oc5ccccc5c4cc3c2)c2ccc3c4ccccc4n(-c4ccccc4)c3c2)cc1]

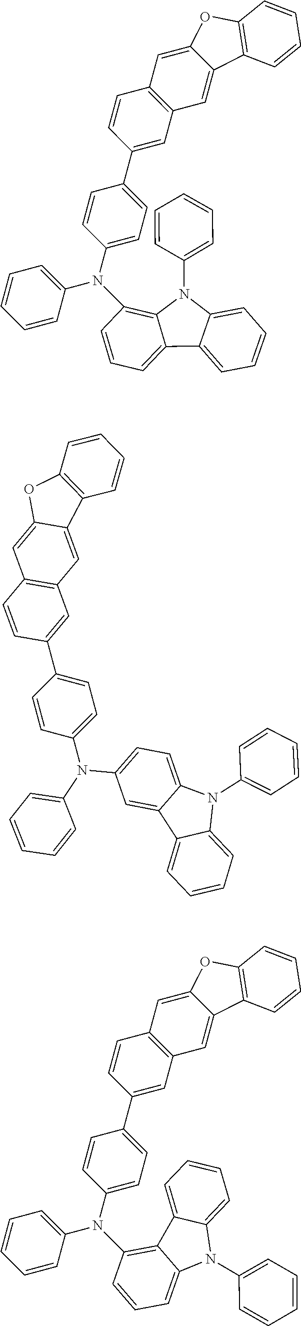 [SMILES: c1ccc(N(c2ccc(-c3ccc4cc5oc6ccccc6c5cc4c3)cc2)c2cccc3c4ccccc4n(-c4ccccc4)c23)cc1 ; c1ccc(N(c2ccc(-c3ccc4cc5oc6ccccc6c5cc4c3)cc2)c2ccc3c(c2)c2ccccc2n3-c2ccccc2)cc1 ; c1ccc(N(c2ccc(-c3ccc4cc5oc6ccccc6c5cc4c3)cc2)c2cccc3c2c2ccccc2n3-c2ccccc2)cc1]

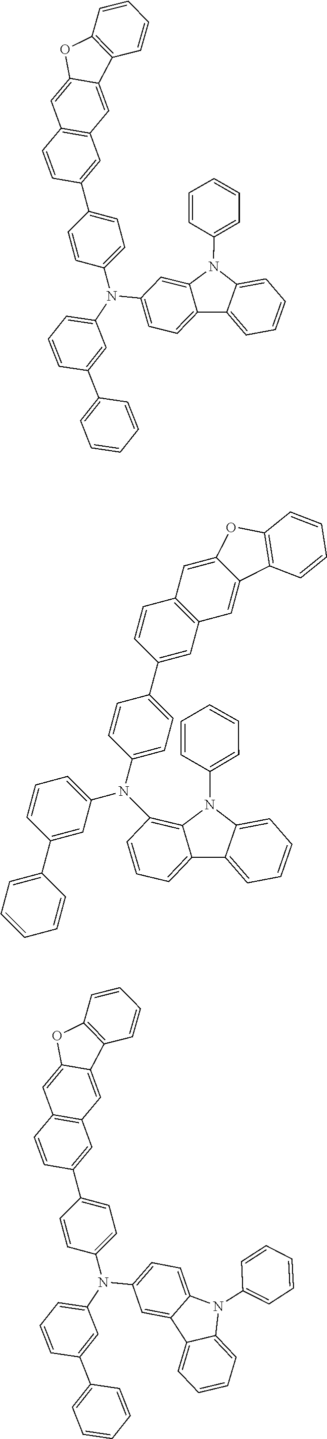 [SMILES: c1ccc(-c2cccc(N(c3ccc(-c4ccc5cc6oc7ccccc7c6cc5c4)cc3)c3ccc4c5ccccc5n(-c5ccccc5)c4c3)c2)cc1 ; c1ccc(-c2cccc(N(c3ccc(-c4ccc5cc6oc7ccccc7c6cc5c4)cc3)c3cccc4c5ccccc5n(-c5ccccc5)c34)c2)cc1 ; c1ccc(-c2cccc(N(c3ccc(-c4ccc5cc6oc7ccccc7c6cc5c4)cc3)c3ccc4c(c3)c3ccccc3n4-c3ccccc3)c2)cc1]

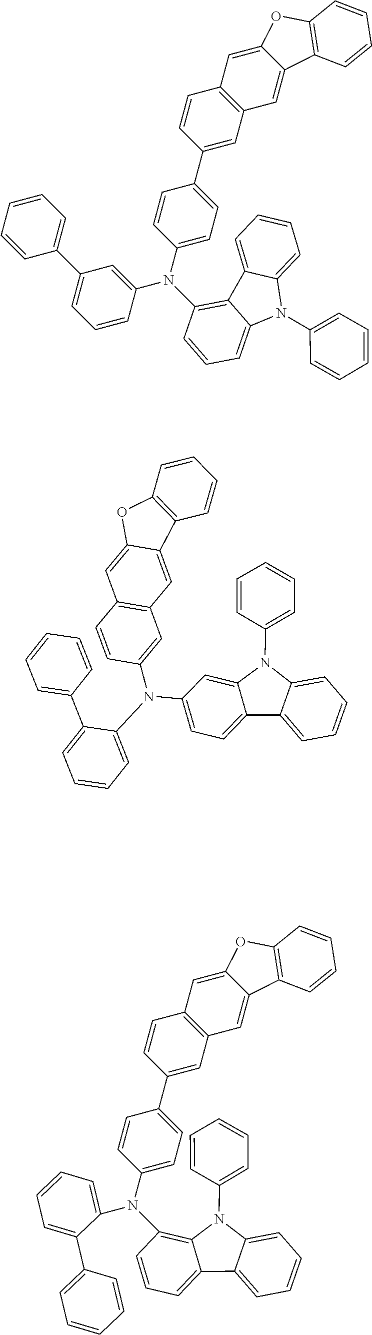 [SMILES: c1ccc(-c2cccc(N(c3ccc(-c4ccc5cc6oc7ccccc7c6cc5c4)cc3)c3cccc4c3c3ccccc3n4-c3ccccc3)c2)cc1 ; c1ccc(-c2ccccc2N(c2ccc3cc4oc5ccccc5c4cc3c2)c2ccc3c4ccccc4n(-c4ccccc4)c3c2)cc1 ; c1ccc(-c2ccccc2N(c2ccc(-c3ccc4cc5oc6ccccc6c5cc4c3)cc2)c2cccc3c4ccccc4n(-c4ccccc4)c23)cc1]

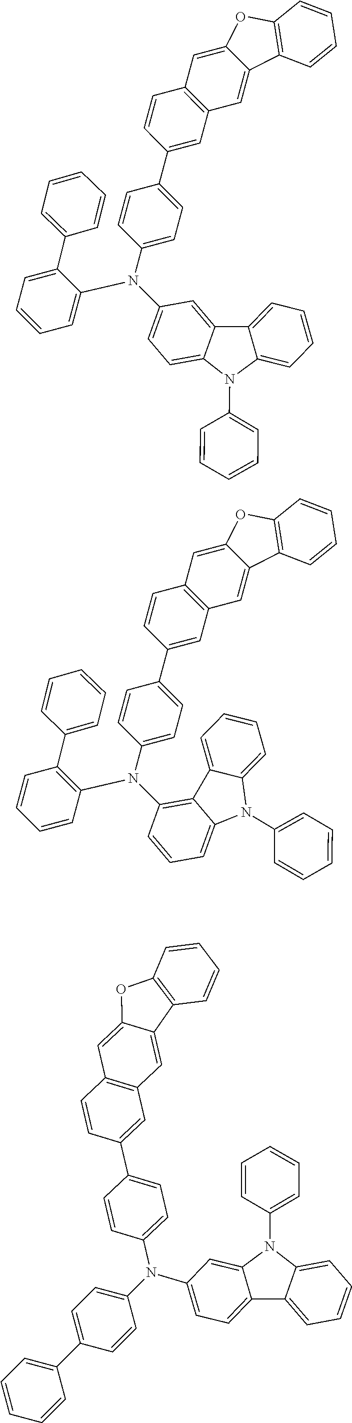

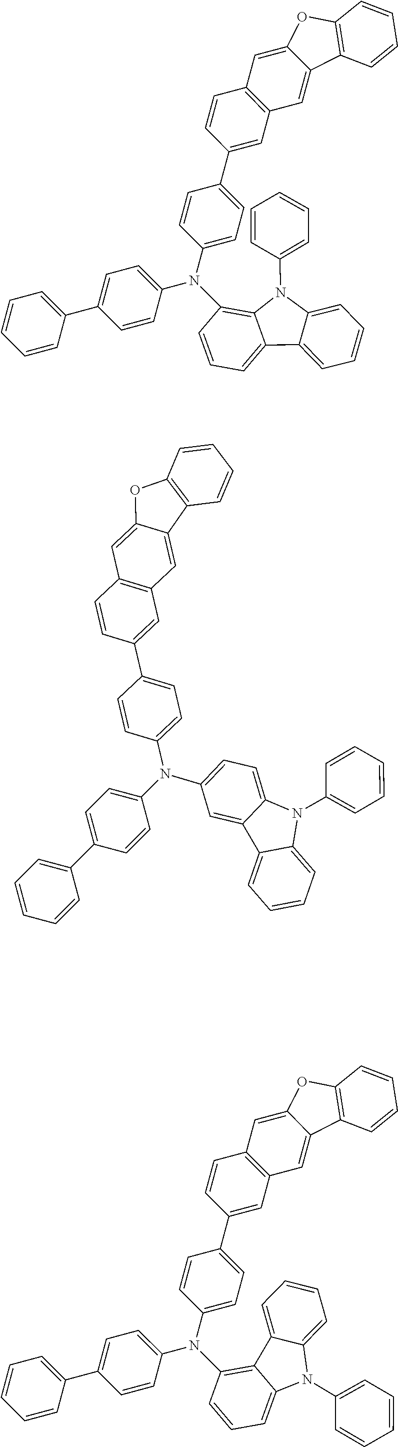 [SMILES: c1ccc(-c2ccc(N(c3ccc(-c4ccc5cc6oc7ccccc7c6cc5c4)cc3)c3cccc4c5ccccc5n(-c5ccccc5)c34)cc2)cc1 ; c1ccc(-c2ccc(N(c3ccc(-c4ccc5cc6oc7ccccc7c6cc5c4)cc3)c3ccc4c(c3)c3ccccc3n4-c3ccccc3)cc2)cc1 ; c1ccc(-c2ccc(N(c3ccc(-c4ccc5cc6oc7ccccc7c6cc5c4)cc3)c3cccc4c3c3ccccc3n4-c3ccccc3)cc2)cc1]

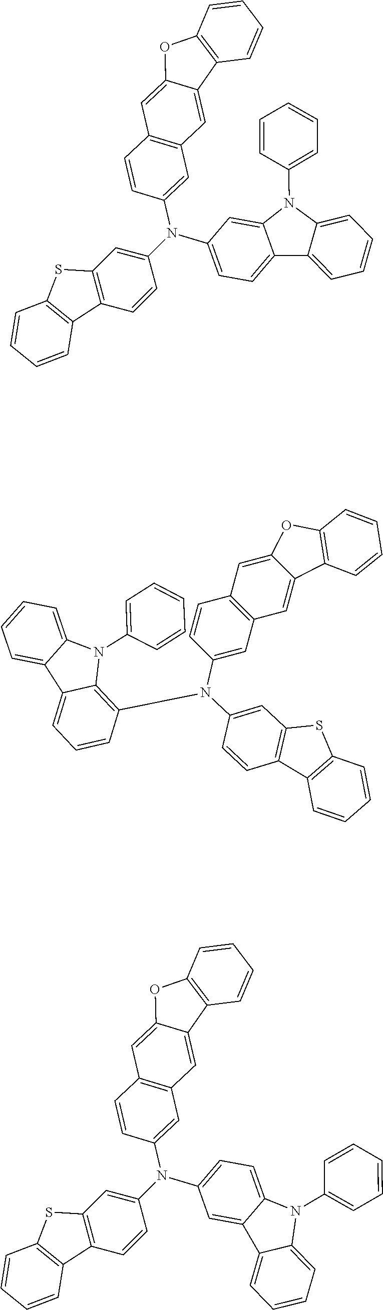 [SMILES: c1ccc(-n2c3ccccc3c3ccc(N(c4ccc5cc6oc7ccccc7c6cc5c4)c4ccc5c(c4)sc4ccccc45)cc32)cc1 ; c1ccc(-n2c3ccccc3c3cccc(N(c4ccc5cc6oc7ccccc7c6cc5c4)c4ccc5c(c4)sc4ccccc45)c32)cc1 ; c1ccc(-n2c3ccccc3c3cc(N(c4ccc5cc6oc7ccccc7c6cc5c4)c4ccc5c(c4)sc4ccccc45)ccc32)cc1]

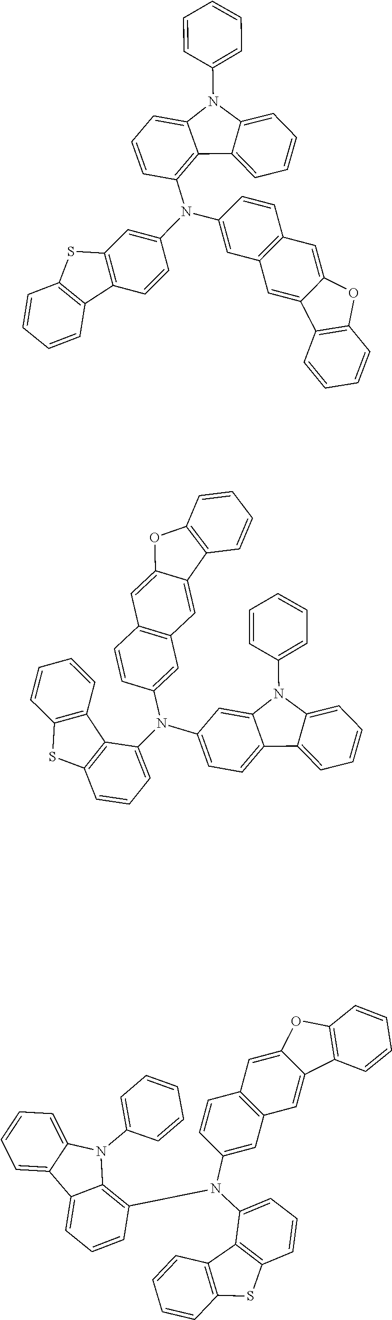 [SMILES: c1ccc(-n2c3ccccc3c3c(N(c4ccc5cc6oc7ccccc7c6cc5c4)c4ccc5c(c4)sc4ccccc45)cccc32)cc1 ; c1ccc(-n2c3ccccc3c3ccc(N(c4ccc5cc6oc7ccccc7c6cc5c4)c4cccc5sc6ccccc6c45)cc32)cc1 ; c1ccc(-n2c3ccccc3c3cccc(N(c4ccc5cc6oc7ccccc7c6cc5c4)c4cccc5sc6ccccc6c45)c32)cc1]

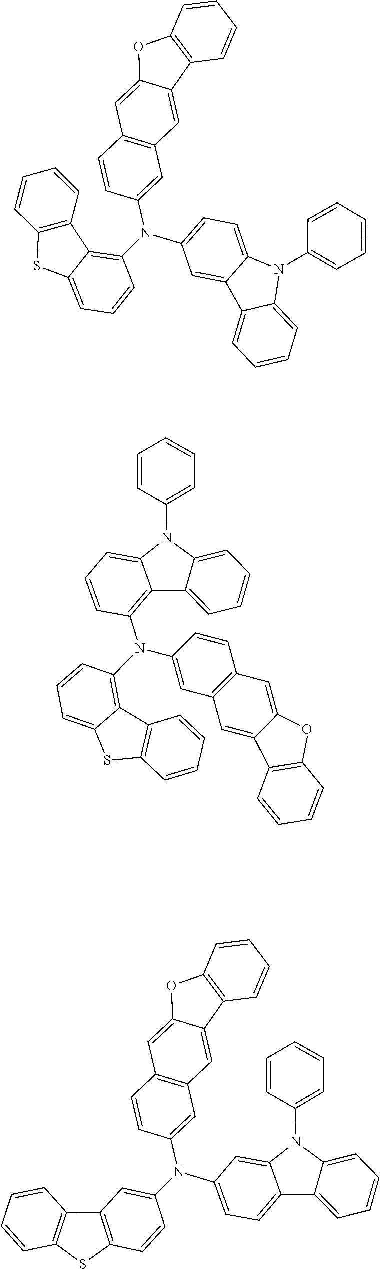 [SMILES: c1ccc(-n2c3ccccc3c3cc(N(c4ccc5cc6oc7ccccc7c6cc5c4)c4cccc5sc6ccccc6c45)ccc32)cc1 ; c1ccc(-n2c3ccccc3c3c(N(c4ccc5cc6oc7ccccc7c6cc5c4)c4cccc5sc6ccccc6c45)cccc32)cc1 ; c1ccc(-n2c3ccccc3c3ccc(N(c4ccc5cc6oc7ccccc7c6cc5c4)c4ccc5sc6ccccc6c5c4)cc32)cc1]

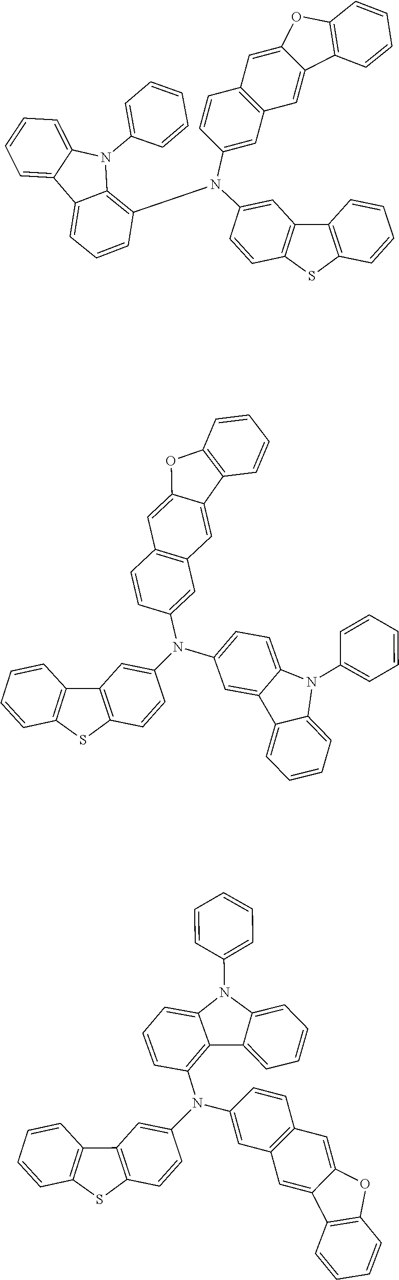 [SMILES: c1ccc(-n2c3ccccc3c3cccc(N(c4ccc5cc6oc7ccccc7c6cc5c4)c4ccc5sc6ccccc6c5c4)c32)cc1 ; c1ccc(-n2c3ccccc3c3cc(N(c4ccc5cc6oc7ccccc7c6cc5c4)c4ccc5sc6ccccc6c5c4)ccc32)cc1 ; c1ccc(-n2c3ccccc3c3c(N(c4ccc5cc6oc7ccccc7c6cc5c4)c4ccc5sc6ccccc6c5c4)cccc32)cc1]

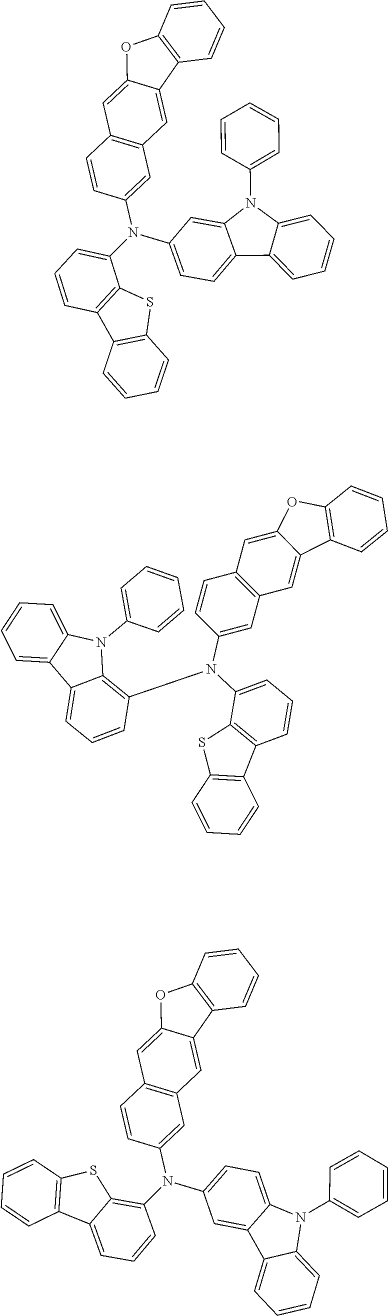 [SMILES: c1ccc(-n2c3ccccc3c3ccc(N(c4ccc5cc6oc7ccccc7c6cc5c4)c4cccc5c4sc4ccccc45)cc32)cc1 ; c1ccc(-n2c3ccccc3c3cccc(N(c4ccc5cc6oc7ccccc7c6cc5c4)c4cccc5c4sc4ccccc45)c32)cc1 ; c1ccc(-n2c3ccccc3c3cc(N(c4ccc5cc6oc7ccccc7c6cc5c4)c4cccc5c4sc4ccccc45)ccc32)cc1]

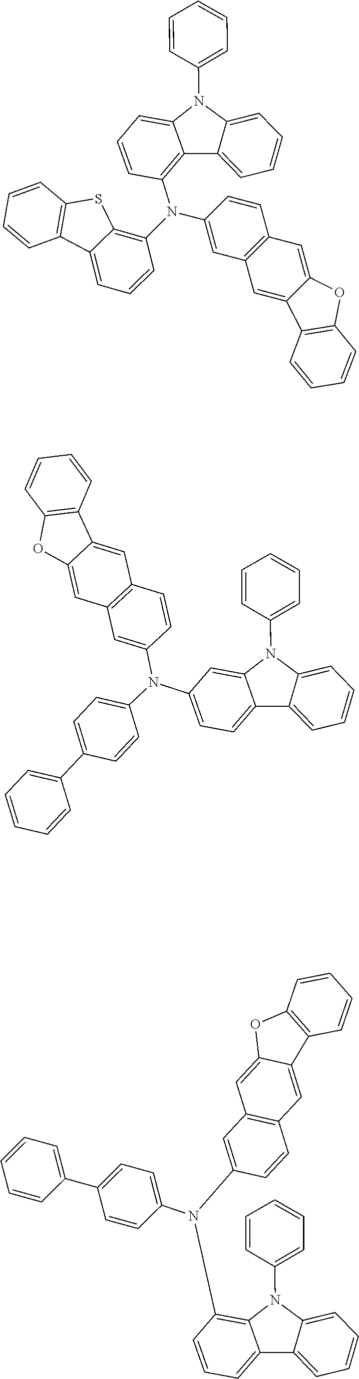 [SMILES: c1ccc(-n2c3ccccc3c3c(N(c4ccc5cc6oc7ccccc7c6cc5c4)c4cccc5c4sc4ccccc45)cccc32)cc1 ; c1ccc(-c2ccc(N(c3ccc4cc5c(cc4c3)oc3ccccc35)c3ccc4c5ccccc5n(-c5ccccc5)c4c3)cc2)cc1 ; c1ccc(-c2ccc(N(c3ccc4cc5c(cc4c3)oc3ccccc35)c3cccc4c5ccccc5n(-c5ccccc5)c34)cc2)cc1]

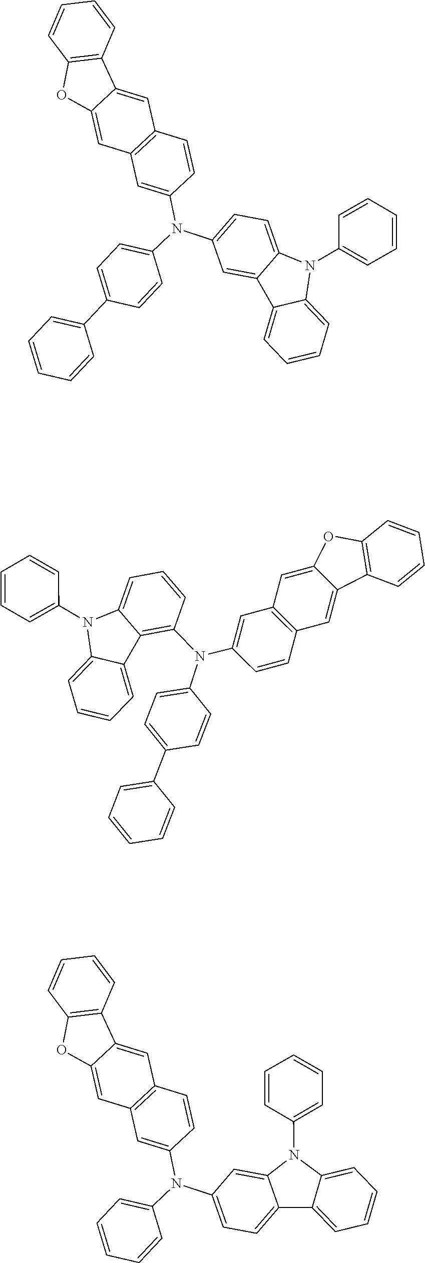 [SMILES: c1ccc(-c2ccc(N(c3ccc4cc5c(cc4c3)oc3ccccc35)c3ccc4c(c3)c3ccccc3n4-c3ccccc3)cc2)cc1 ; c1ccc(-c2ccc(N(c3ccc4cc5c(cc4c3)oc3ccccc35)c3cccc4c3c3ccccc3n4-c3ccccc3)cc2)cc1 ; c1ccc(N(c2ccc3cc4c(cc3c2)oc2ccccc24)c2ccc3c4ccccc4n(-c4ccccc4)c3c2)cc1]

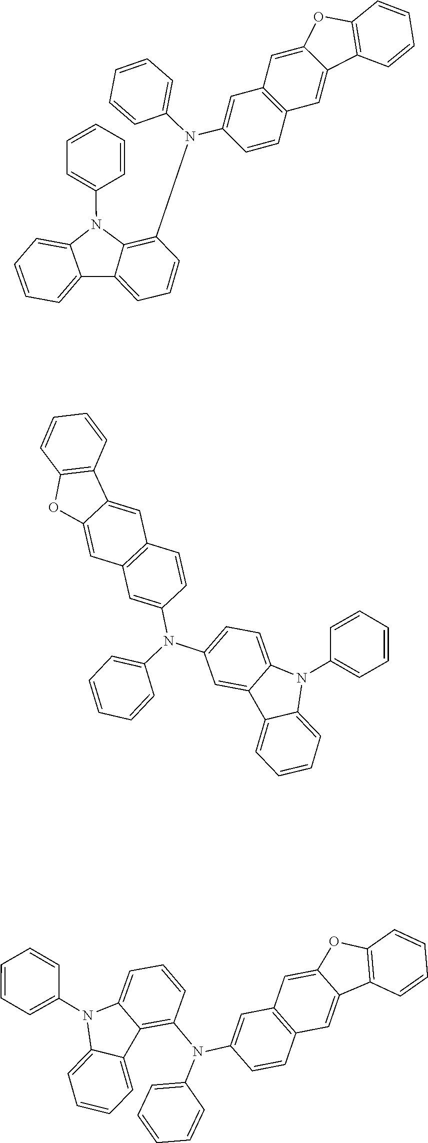 [SMILES: c1ccc(N(c2ccc3cc4c(cc3c2)oc2ccccc24)c2cccc3c4ccccc4n(-c4ccccc4)c23)cc1 ; c1ccc(N(c2ccc3cc4c(cc3c2)oc2ccccc24)c2ccc3c(c2)c2ccccc2n3-c2ccccc2)cc1 ; c1ccc(N(c2ccc3cc4c(cc3c2)oc2ccccc24)c2cccc3c2c2ccccc2n3-c2ccccc2)cc1]

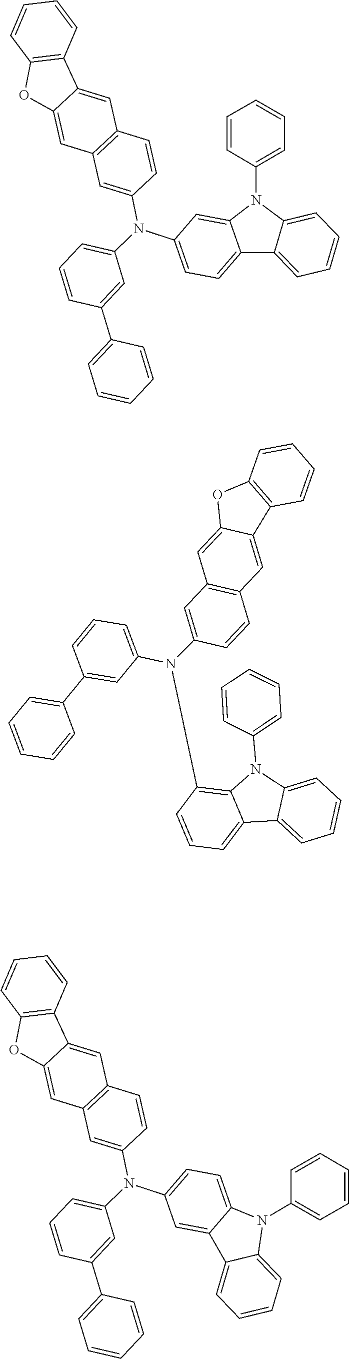 [SMILES: c1ccc(-c2cccc(N(c3ccc4cc5c(cc4c3)oc3ccccc35)c3ccc4c5ccccc5n(-c5ccccc5)c4c3)c2)cc1 ; c1ccc(-c2cccc(N(c3ccc4cc5c(cc4c3)oc3ccccc35)c3cccc4c5ccccc5n(-c5ccccc5)c34)c2)cc1 ; c1ccc(-c2cccc(N(c3ccc4cc5c(cc4c3)oc3ccccc35)c3ccc4c(c3)c3ccccc3n4-c3ccccc3)c2)cc1]

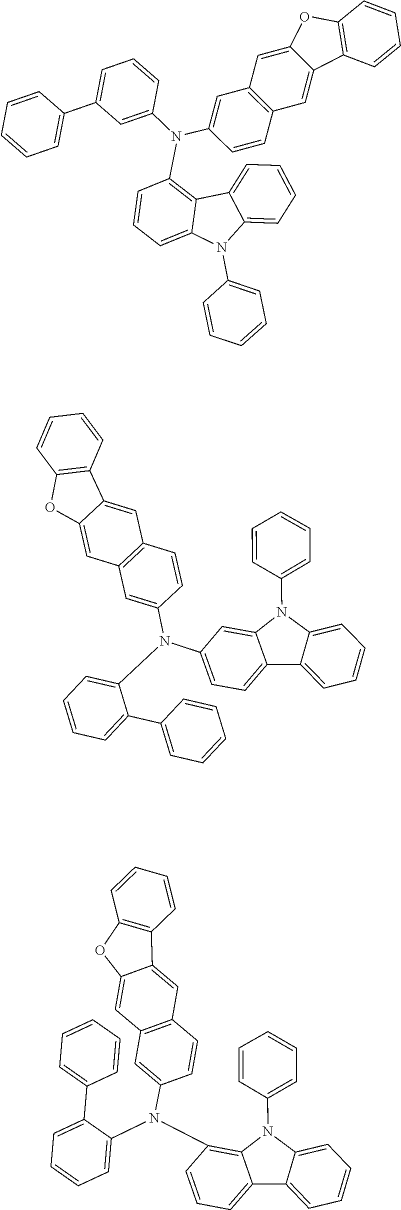 [SMILES: c1ccc(-c2cccc(N(c3ccc4cc5c(cc4c3)oc3ccccc35)c3cccc4c3c3ccccc3n4-c3ccccc3)c2)cc1 ; c1ccc(-c2ccccc2N(c2ccc3cc4c(cc3c2)oc2ccccc24)c2ccc3c4ccccc4n(-c4ccccc4)c3c2)cc1 ; c1ccc(-c2ccccc2N(c2ccc3cc4c(cc3c2)oc2ccccc24)c2cccc3c4ccccc4n(-c4ccccc4)c23)cc1]

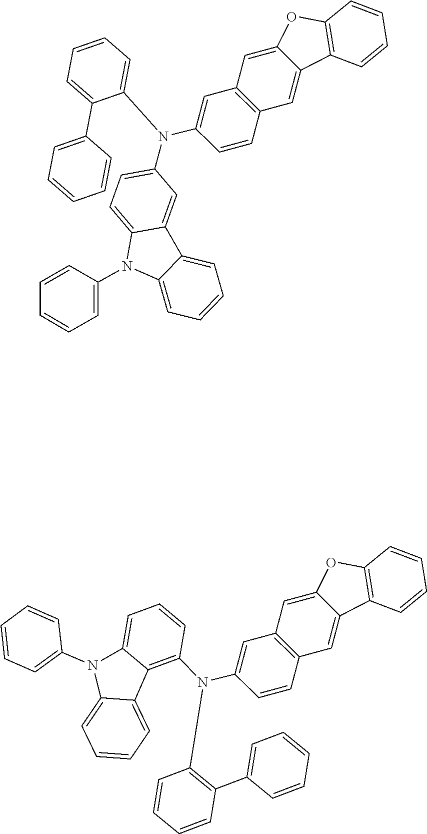 [SMILES: c1ccc(-c2ccccc2N(c2ccc3cc4c(cc3c2)oc2ccccc24)c2ccc3c(c2)c2ccccc2n3-c2ccccc2)cc1 ; c1ccc(-c2ccccc2N(c2ccc3cc4c(cc3c2)oc2ccccc24)c2cccc3c2c2ccccc2n3-c2ccccc2)cc1]

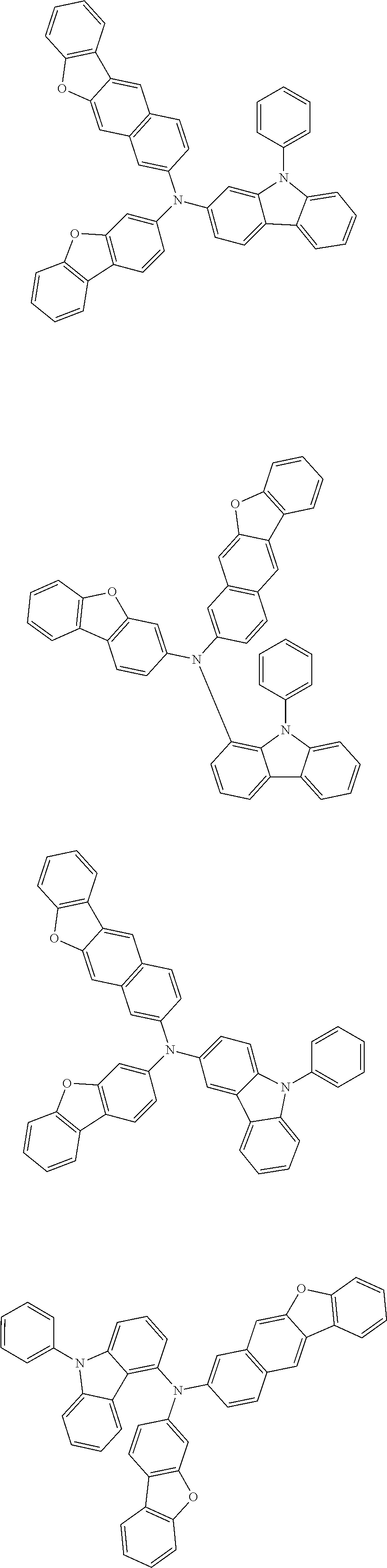 [SMILES: c1ccc(-n2c3ccccc3c3ccc(N(c4ccc5cc6c(cc5c4)oc4ccccc46)c4ccc5c(c4)oc4ccccc45)cc32)cc1 ; c1ccc(-n2c3ccccc3c3cccc(N(c4ccc5cc6c(cc5c4)oc4ccccc46)c4ccc5c(c4)oc4ccccc45)c32)cc1 ; c1ccc(-n2c3ccccc3c3cc(N(c4ccc5cc6c(cc5c4)oc4ccccc46)c4ccc5c(c4)oc4ccccc45)ccc32)cc1 ; c1ccc(-n2c3ccccc3c3c(N(c4ccc5cc6c(cc5c4)oc4ccccc46)c4ccc5c(c4)oc4ccccc45)cccc32)cc1]

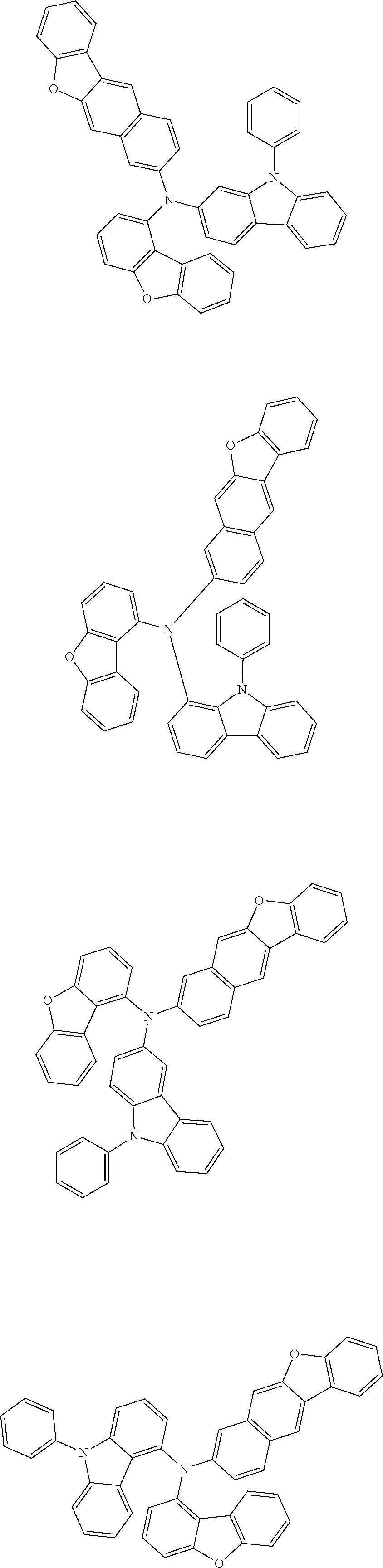 [SMILES: c1ccc(-n2c3ccccc3c3ccc(N(c4ccc5cc6c(cc5c4)oc4ccccc46)c4cccc5oc6ccccc6c45)cc32)cc1 ; c1ccc(-n2c3ccccc3c3cccc(N(c4ccc5cc6c(cc5c4)oc4ccccc46)c4cccc5oc6ccccc6c45)c32)cc1 ; c1ccc(-n2c3ccccc3c3cc(N(c4ccc5cc6c(cc5c4)oc4ccccc46)c4cccc5oc6ccccc6c45)ccc32)cc1 ; c1ccc(-n2c3ccccc3c3c(N(c4ccc5cc6c(cc5c4)oc4ccccc46)c4cccc5oc6ccccc6c45)cccc32)cc1]

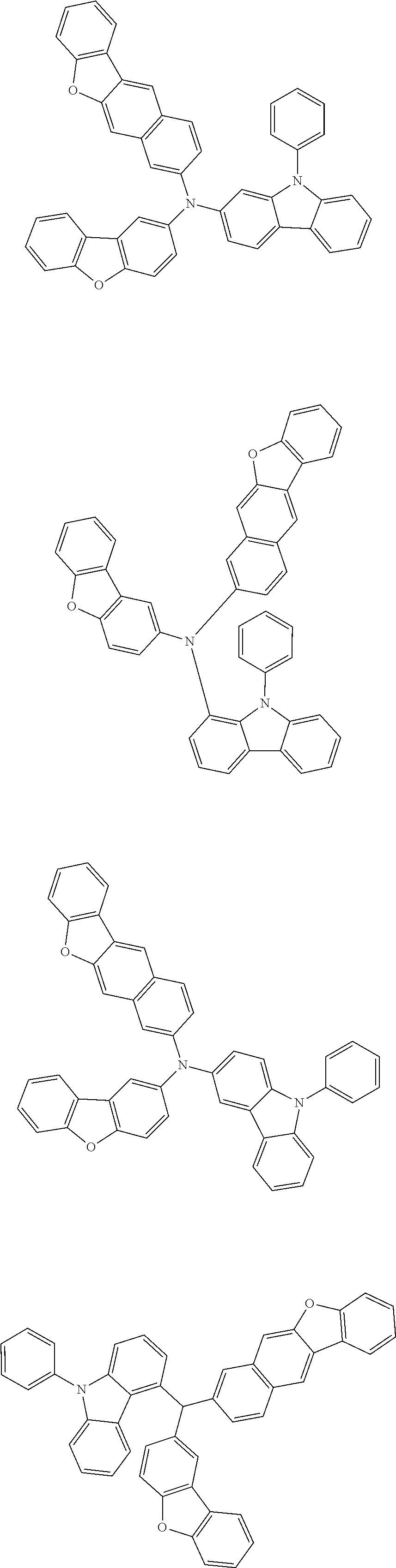 [SMILES: c1ccc(-n2c3ccccc3c3ccc(N(c4ccc5cc6c(cc5c4)oc4ccccc46)c4ccc5oc6ccccc6c5c4)cc32)cc1 ; c1ccc(-n2c3ccccc3c3cccc(N(c4ccc5cc6c(cc5c4)oc4ccccc46)c4ccc5oc6ccccc6c5c4)c32)cc1 ; c1ccc(-n2c3ccccc3c3cc(N(c4ccc5cc6c(cc5c4)oc4ccccc46)c4ccc5oc6ccccc6c5c4)ccc32)cc1 ; c1ccc(-n2c3ccccc3c3c(C(c4ccc5cc6c(cc5c4)oc4ccccc46)c4ccc5oc6ccccc6c5c4)cccc32)cc1]

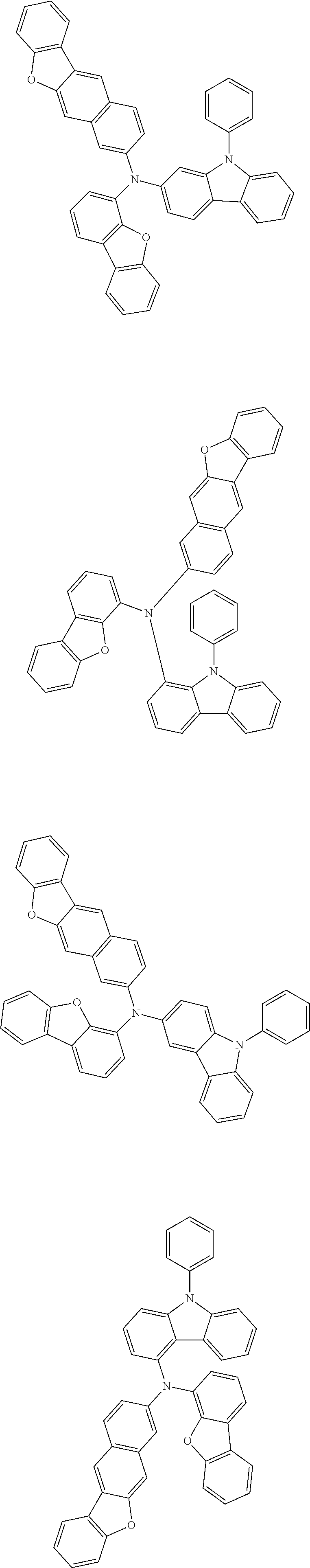 [SMILES: c1ccc(-n2c3ccccc3c3ccc(N(c4ccc5cc6c(cc5c4)oc4ccccc46)c4cccc5c4oc4ccccc45)cc32)cc1 ; c1ccc(-n2c3ccccc3c3cccc(N(c4ccc5cc6c(cc5c4)oc4ccccc46)c4cccc5c4oc4ccccc45)c32)cc1 ; c1ccc(-n2c3ccccc3c3cc(N(c4ccc5cc6c(cc5c4)oc4ccccc46)c4cccc5c4oc4ccccc45)ccc32)cc1 ; c1ccc(-n2c3ccccc3c3c(N(c4ccc5cc6c(cc5c4)oc4ccccc46)c4cccc5c4oc4ccccc45)cccc32)cc1]

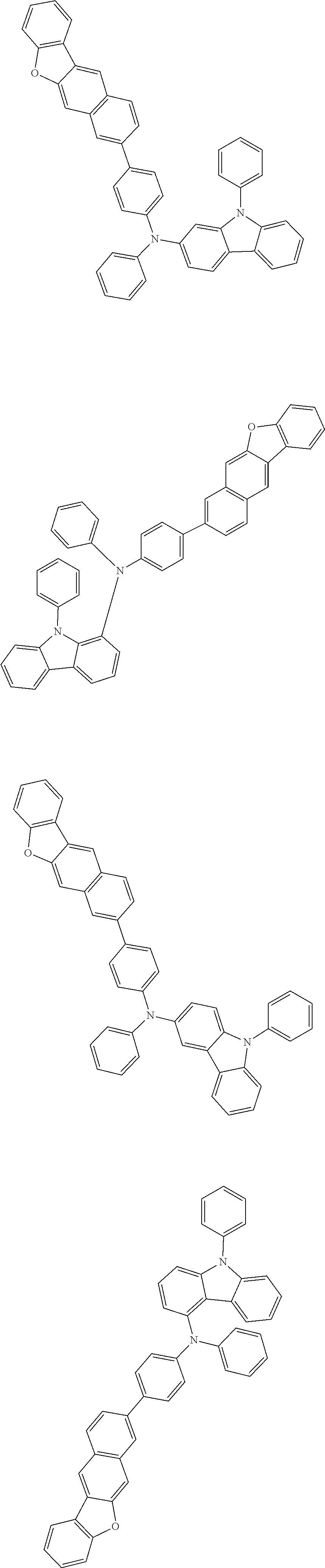 [SMILES: c1ccc(N(c2ccc(-c3ccc4cc5c(cc4c3)oc3ccccc35)cc2)c2ccc3c4ccccc4n(-c4ccccc4)c3c2)cc1 ; c1ccc(N(c2ccc(-c3ccc4cc5c(cc4c3)oc3ccccc35)cc2)c2cccc3c4ccccc4n(-c4ccccc4)c23)cc1 ; c1ccc(N(c2ccc(-c3ccc4cc5c(cc4c3)oc3ccccc35)cc2)c2ccc3c(c2)c2ccccc2n3-c2ccccc2)cc1 ; c1ccc(N(c2ccc(-c3ccc4cc5c(cc4c3)oc3ccccc35)cc2)c2cccc3c2c2ccccc2n3-c2ccccc2)cc1]

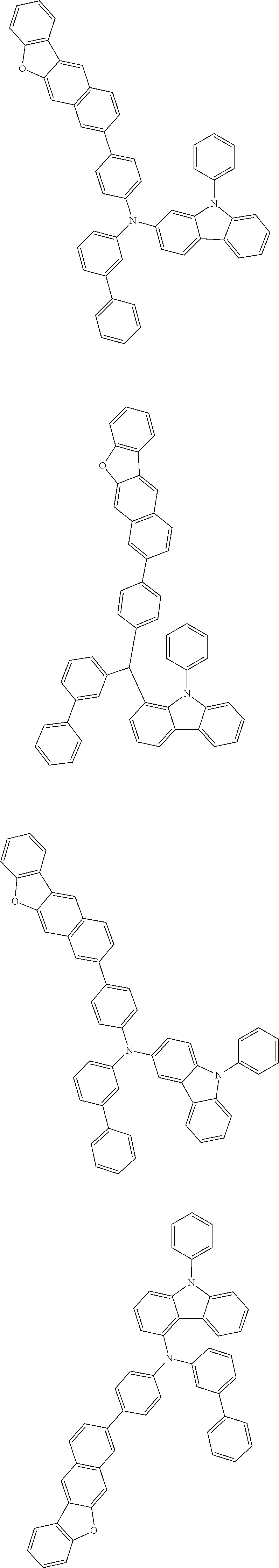 [SMILES: c1ccc(-c2cccc(N(c3ccc(-c4ccc5cc6c(cc5c4)oc4ccccc46)cc3)c3ccc4c5ccccc5n(-c5ccccc5)c4c3)c2)cc1 ; c1ccc(-c2cccc(C(c3ccc(-c4ccc5cc6c(cc5c4)oc4ccccc46)cc3)c3cccc4c5ccccc5n(-c5ccccc5)c34)c2)cc1 ; c1ccc(-c2cccc(N(c3ccc(-c4ccc5cc6c(cc5c4)oc4ccccc46)cc3)c3ccc4c(c3)c3ccccc3n4-c3ccccc3)c2)cc1 ; c1ccc(-c2cccc(N(c3ccc(-c4ccc5cc6c(cc5c4)oc4ccccc46)cc3)c3cccc4c3c3ccccc3n4-c3ccccc3)c2)cc1]

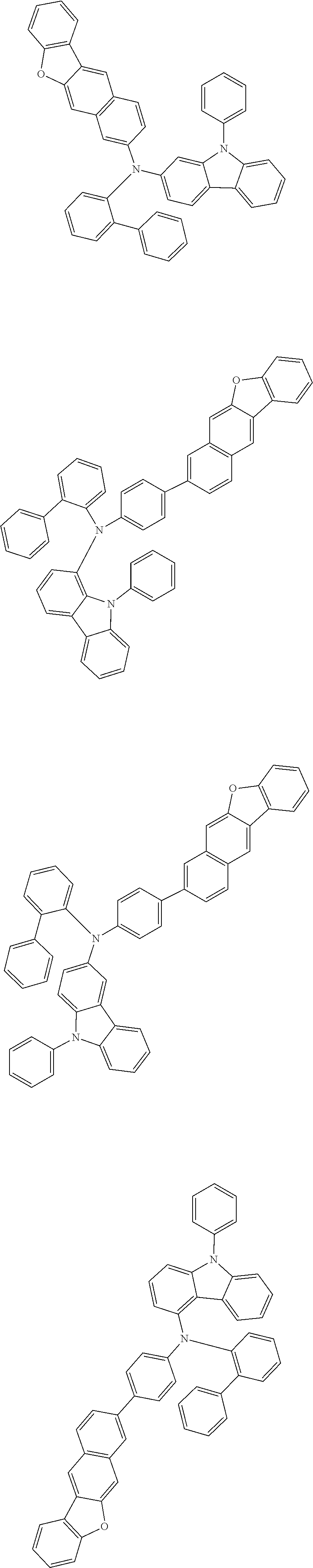 [SMILES: c1ccc(-c2ccccc2N(c2ccc3cc4c(cc3c2)oc2ccccc24)c2ccc3c4ccccc4n(-c4ccccc4)c3c2)cc1 ; c1ccc(-c2ccccc2N(c2ccc(-c3ccc4cc5c(cc4c3)oc3ccccc35)cc2)c2cccc3c4ccccc4n(-c4ccccc4)c23)cc1 ; c1ccc(-c2ccccc2N(c2ccc(-c3ccc4cc5c(cc4c3)oc3ccccc35)cc2)c2ccc3c(c2)c2ccccc2n3-c2ccccc2)cc1 ; c1ccc(-c2ccccc2N(c2ccc(-c3ccc4cc5c(cc4c3)oc3ccccc35)cc2)c2cccc3c2c2ccccc2n3-c2ccccc2)cc1]

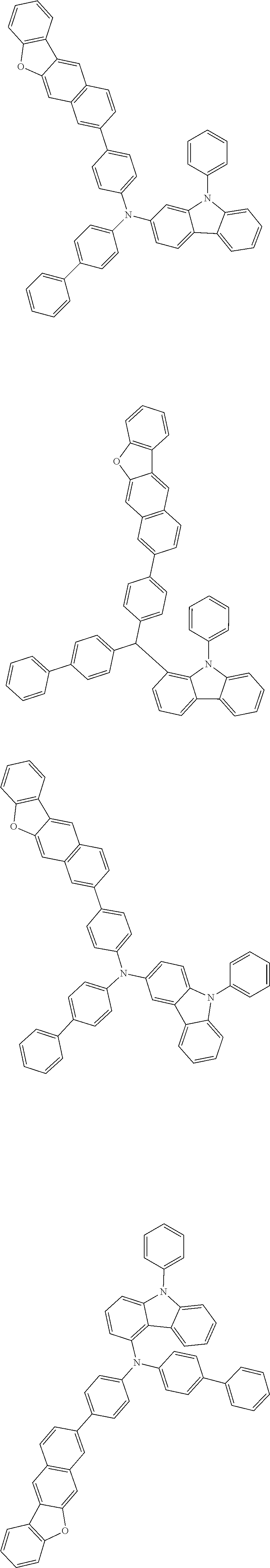 [SMILES: c1ccc(-c2ccc(N(c3ccc(-c4ccc5cc6c(cc5c4)oc4ccccc46)cc3)c3ccc4c5ccccc5n(-c5ccccc5)c4c3)cc2)cc1 ; c1ccc(-c2ccc(C(c3ccc(-c4ccc5cc6c(cc5c4)oc4ccccc46)cc3)c3cccc4c5ccccc5n(-c5ccccc5)c34)cc2)cc1 ; c1ccc(-c2ccc(N(c3ccc(-c4ccc5cc6c(cc5c4)oc4ccccc46)cc3)c3ccc4c(c3)c3ccccc3n4-c3ccccc3)cc2)cc1 ; c1ccc(-c2ccc(N(c3ccc(-c4ccc5cc6c(cc5c4)oc4ccccc46)cc3)c3cccc4c3c3ccccc3n4-c3ccccc3)cc2)cc1]

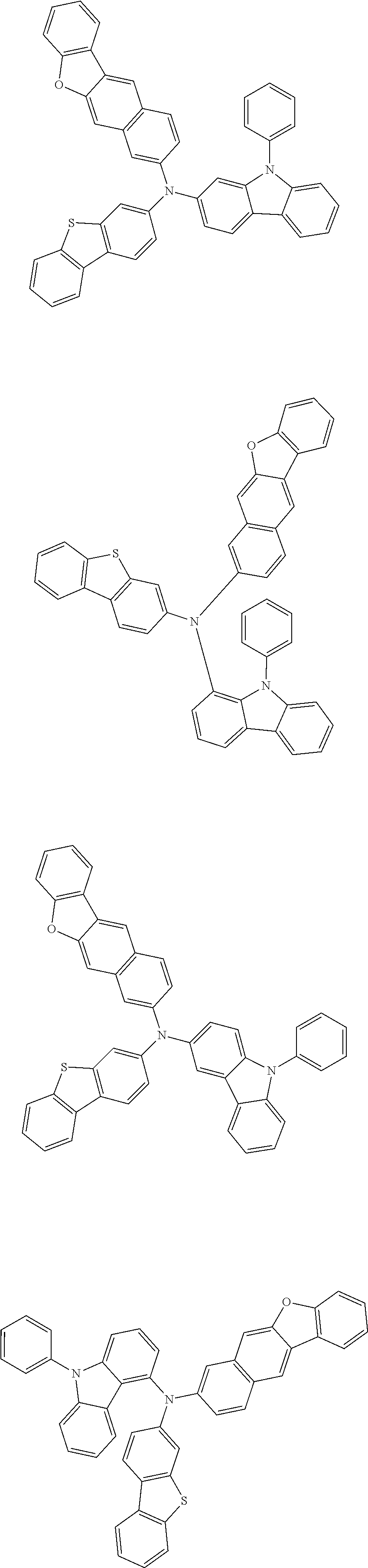 [SMILES: c1ccc(-n2c3ccccc3c3ccc(N(c4ccc5cc6c(cc5c4)oc4ccccc46)c4ccc5c(c4)sc4ccccc45)cc32)cc1 ; c1ccc(-n2c3ccccc3c3cccc(N(c4ccc5cc6c(cc5c4)oc4ccccc46)c4ccc5c(c4)sc4ccccc45)c32)cc1 ; c1ccc(-n2c3ccccc3c3cc(N(c4ccc5cc6c(cc5c4)oc4ccccc46)c4ccc5c(c4)sc4ccccc45)ccc32)cc1 ; c1ccc(-n2c3ccccc3c3c(N(c4ccc5cc6c(cc5c4)oc4ccccc46)c4ccc5c(c4)sc4ccccc45)cccc32)cc1]

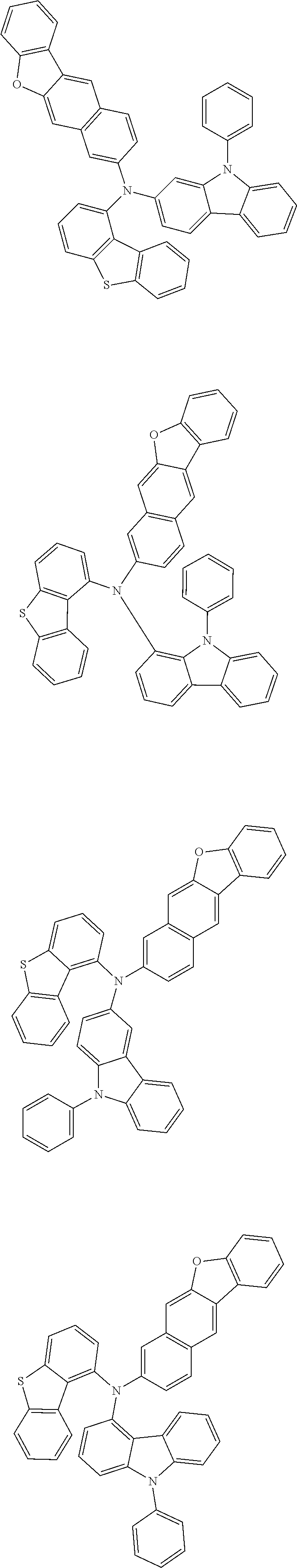 [SMILES: c1ccc(-n2c3ccccc3c3ccc(N(c4ccc5cc6c(cc5c4)oc4ccccc46)c4cccc5sc6ccccc6c45)cc32)cc1 ; c1ccc(-n2c3ccccc3c3cccc(N(c4ccc5cc6c(cc5c4)oc4ccccc46)c4cccc5sc6ccccc6c45)c32)cc1 ; c1ccc(-n2c3ccccc3c3cc(N(c4ccc5cc6c(cc5c4)oc4ccccc46)c4cccc5sc6ccccc6c45)ccc32)cc1 ; c1ccc(-n2c3ccccc3c3c(N(c4ccc5cc6c(cc5c4)oc4ccccc46)c4cccc5sc6ccccc6c45)cccc32)cc1]

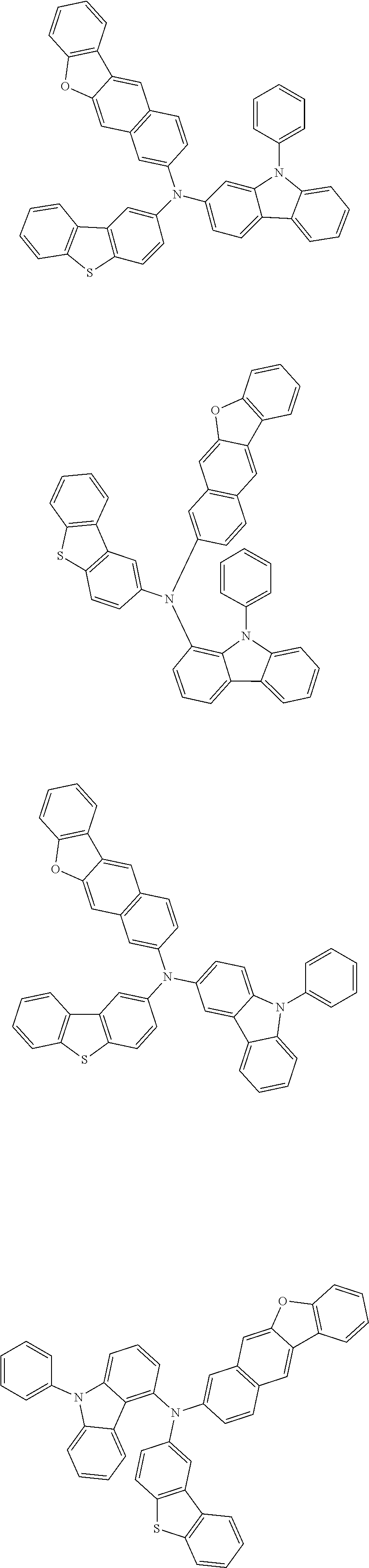 [SMILES: c1ccc(-n2c3ccccc3c3ccc(N(c4ccc5cc6c(cc5c4)oc4ccccc46)c4ccc5sc6ccccc6c5c4)cc32)cc1 ; c1ccc(-n2c3ccccc3c3cccc(N(c4ccc5cc6c(cc5c4)oc4ccccc46)c4ccc5sc6ccccc6c5c4)c32)cc1 ; c1ccc(-n2c3ccccc3c3cc(N(c4ccc5cc6c(cc5c4)oc4ccccc46)c4ccc5sc6ccccc6c5c4)ccc32)cc1 ; c1ccc(-n2c3ccccc3c3c(N(c4ccc5cc6c(cc5c4)oc4ccccc46)c4ccc5sc6ccccc6c5c4)cccc32)cc1]

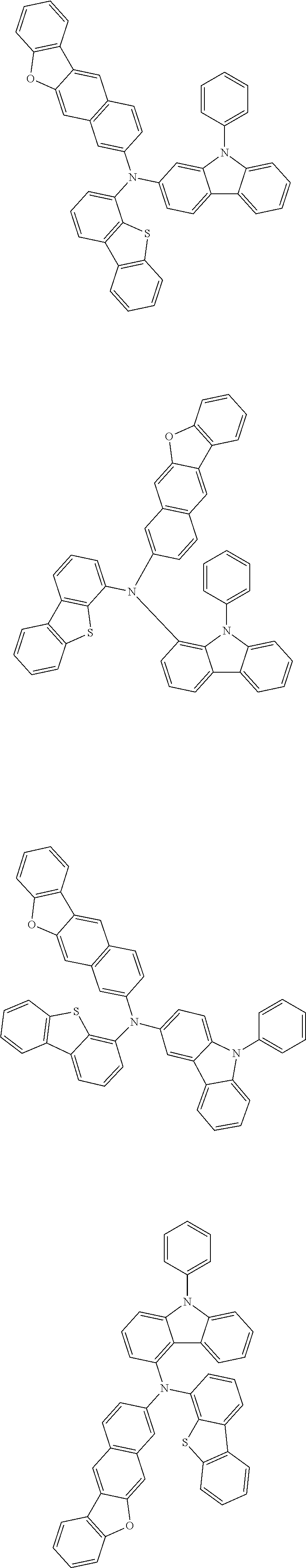 [SMILES: c1ccc(-n2c3ccccc3c3ccc(N(c4ccc5cc6c(cc5c4)oc4ccccc46)c4cccc5c4sc4ccccc45)cc32)cc1 ; c1ccc(-n2c3ccccc3c3cccc(N(c4ccc5cc6c(cc5c4)oc4ccccc46)c4cccc5c4sc4ccccc45)c32)cc1 ; c1ccc(-n2c3ccccc3c3cc(N(c4ccc5cc6c(cc5c4)oc4ccccc46)c4cccc5c4sc4ccccc45)ccc32)cc1 ; c1ccc(-n2c3ccccc3c3c(N(c4ccc5cc6c(cc5c4)oc4ccccc46)c4cccc5c4sc4ccccc45)cccc32)cc1]

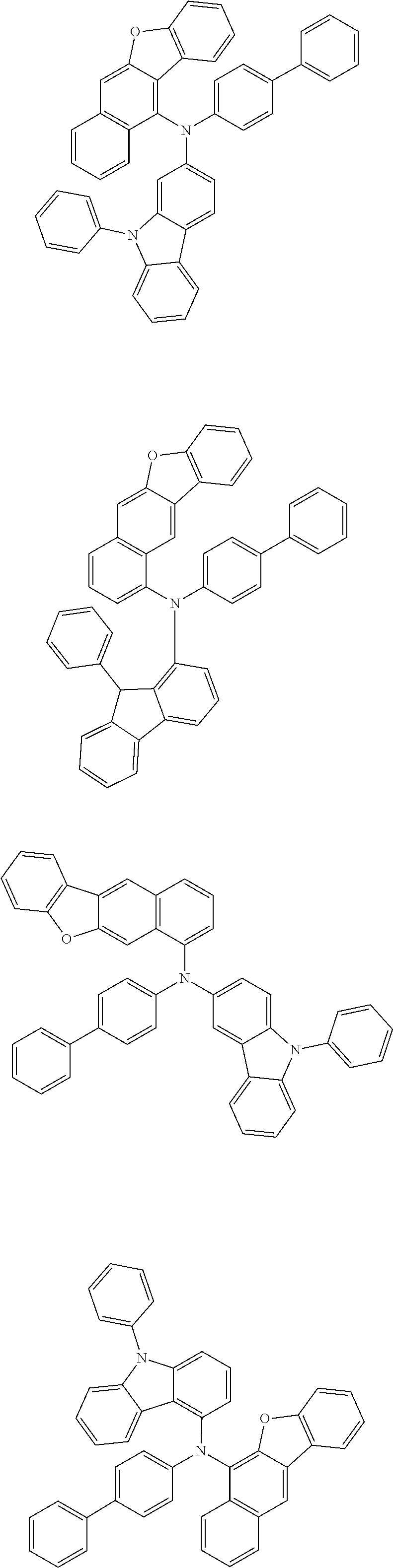 [SMILES: c1ccc(-c2ccc(N(c3ccc4c5ccccc5n(-c5ccccc5)c4c3)c3c4ccccc4cc4oc5ccccc5c34)cc2)cc1 ; c1ccc(-c2ccc(N(c3cccc4c3C(c3ccccc3)c3ccccc3-4)c3cccc4cc5oc6ccccc6c5cc34)cc2)cc1 ; c1ccc(-c2ccc(N(c3ccc4c(c3)c3ccccc3n4-c3ccccc3)c3cccc4cc5c(cc34)oc3ccccc35)cc2)cc1 ; c1ccc(-c2ccc(N(c3c4ccccc4cc4c3oc3ccccc34)c3cccc4c3c3ccccc3n4-c3ccccc3)cc2)cc1]

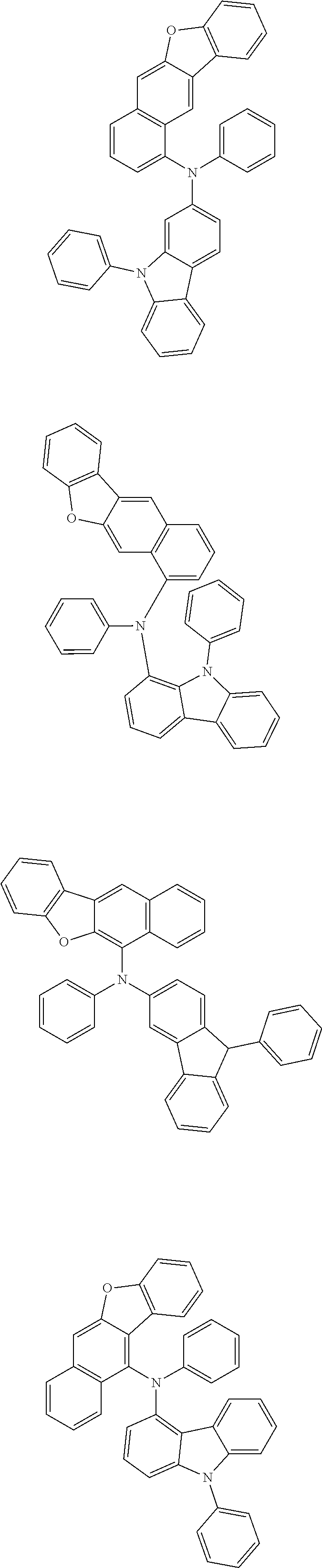 [SMILES: c1ccc(N(c2ccc3c4ccccc4n(-c4ccccc4)c3c2)c2cccc3cc4oc5ccccc5c4cc23)cc1 ; c1ccc(N(c2cccc3cc4c(cc23)oc2ccccc24)c2cccc3c4ccccc4n(-c4ccccc4)c23)cc1 ; c1ccc(C2c3ccccc3-c3cc(N(c4ccccc4)c4c5ccccc5cc5c4oc4ccccc45)ccc32)cc1 ; c1ccc(N(c2c3ccccc3cc3oc4ccccc4c23)c2cccc3c2c2ccccc2n3-c2ccccc2)cc1]

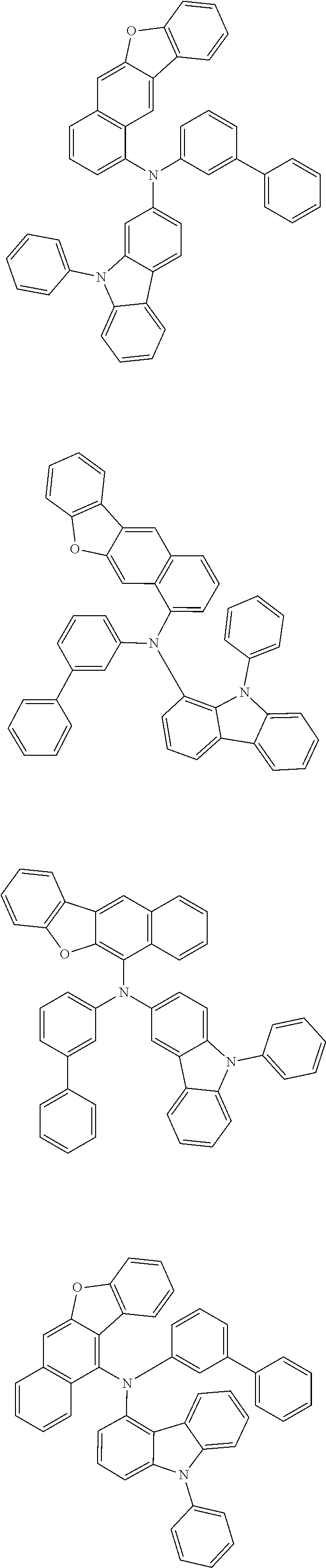 [SMILES: c1ccc(-c2cccc(N(c3ccc4c5ccccc5n(-c5ccccc5)c4c3)c3cccc4cc5oc6ccccc6c5cc34)c2)cc1 ; c1ccc(-c2cccc(N(c3cccc4cc5c(cc34)oc3ccccc35)c3cccc4c5ccccc5n(-c5ccccc5)c34)c2)cc1 ; c1ccc(-c2cccc(N(c3ccc4c(c3)c3ccccc3n4-c3ccccc3)c3c4ccccc4cc4c3oc3ccccc34)c2)cc1 ; c1ccc(-c2cccc(N(c3c4ccccc4cc4oc5ccccc5c34)c3cccc4c3c3ccccc3n4-c3ccccc3)c2)cc1]

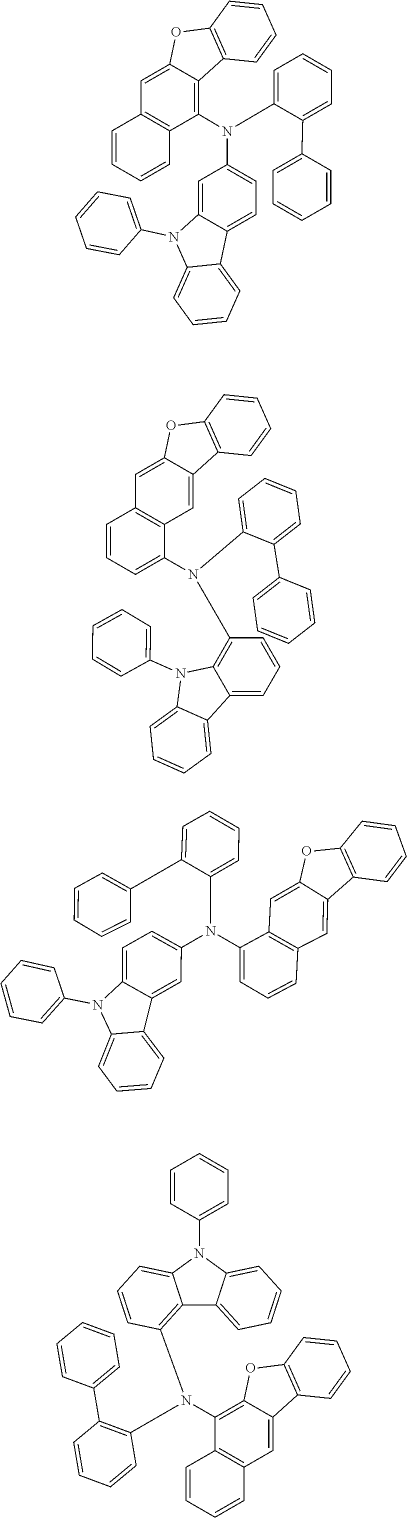

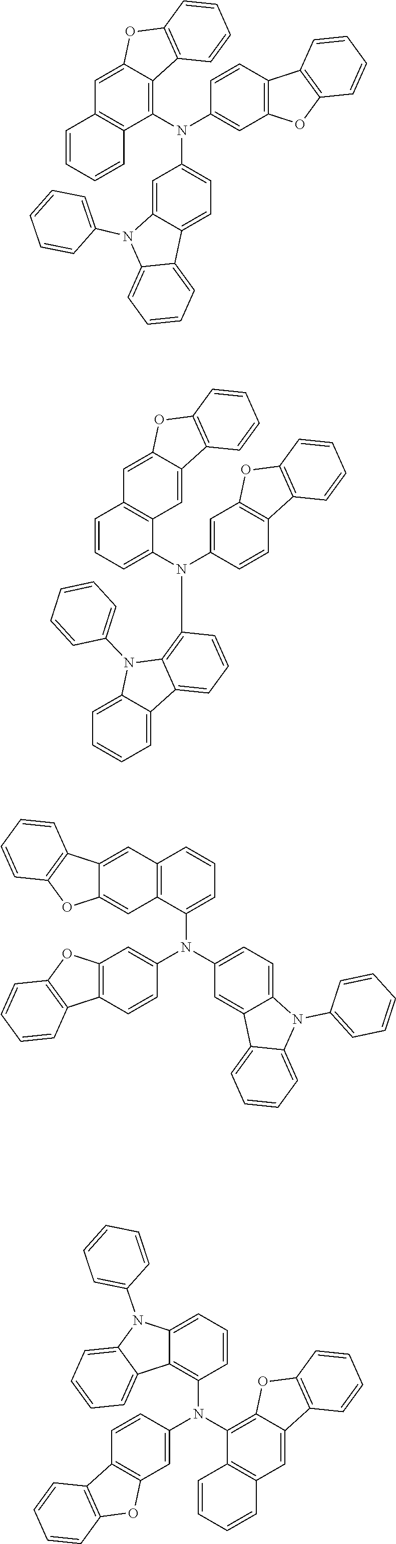 [SMILES: c1ccc(-n2c3ccccc3c3ccc(N(c4ccc5c(c4)oc4ccccc45)c4c5ccccc5cc5oc6ccccc6c45)cc32)cc1 ; c1ccc(-n2c3ccccc3c3cccc(N(c4ccc5c(c4)oc4ccccc45)c4cccc5cc6oc7ccccc7c6cc45)c32)cc1 ; c1ccc(-n2c3ccccc3c3cc(N(c4ccc5c(c4)oc4ccccc45)c4cccc5cc6c(cc45)oc4ccccc46)ccc32)cc1 ; c1ccc(-n2c3ccccc3c3c(N(c4ccc5c(c4)oc4ccccc45)c4c5ccccc5cc5c4oc4ccccc45)cccc32)cc1]

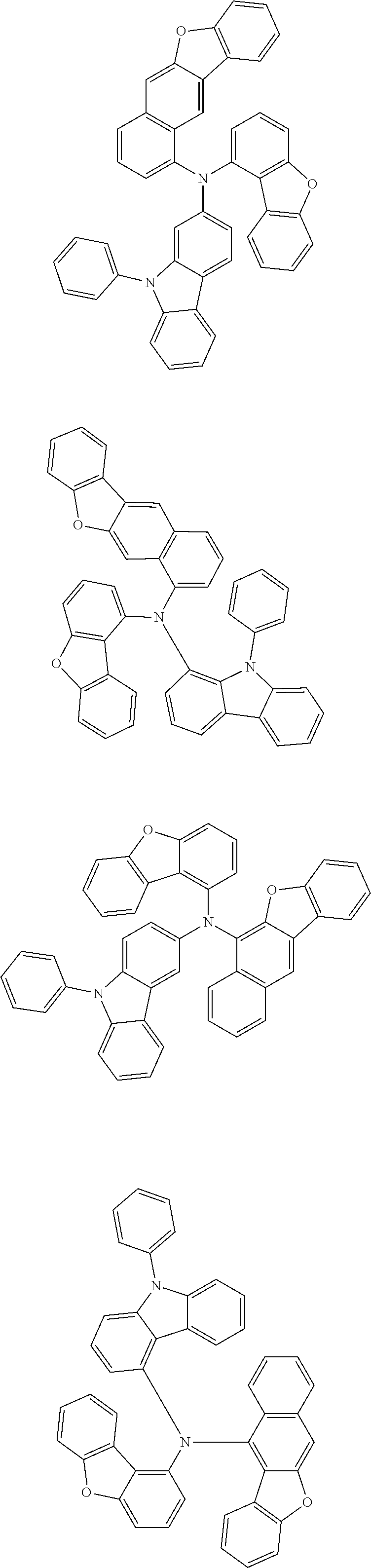 [SMILES: c1ccc(-n2c3ccccc3c3ccc(N(c4cccc5cc6oc7ccccc7c6cc45)c4cccc5oc6ccccc6c45)cc32)cc1 ; c1ccc(-n2c3ccccc3c3cccc(N(c4cccc5cc6c(cc45)oc4ccccc46)c4cccc5oc6ccccc6c45)c32)cc1 ; c1ccc(-n2c3ccccc3c3cc(N(c4c5ccccc5cc5c4oc4ccccc45)c4cccc5oc6ccccc6c45)ccc32)cc1 ; c1ccc(-n2c3ccccc3c3c(N(c4cccc5oc6ccccc6c45)c4c5ccccc5cc5oc6ccccc6c45)cccc32)cc1]

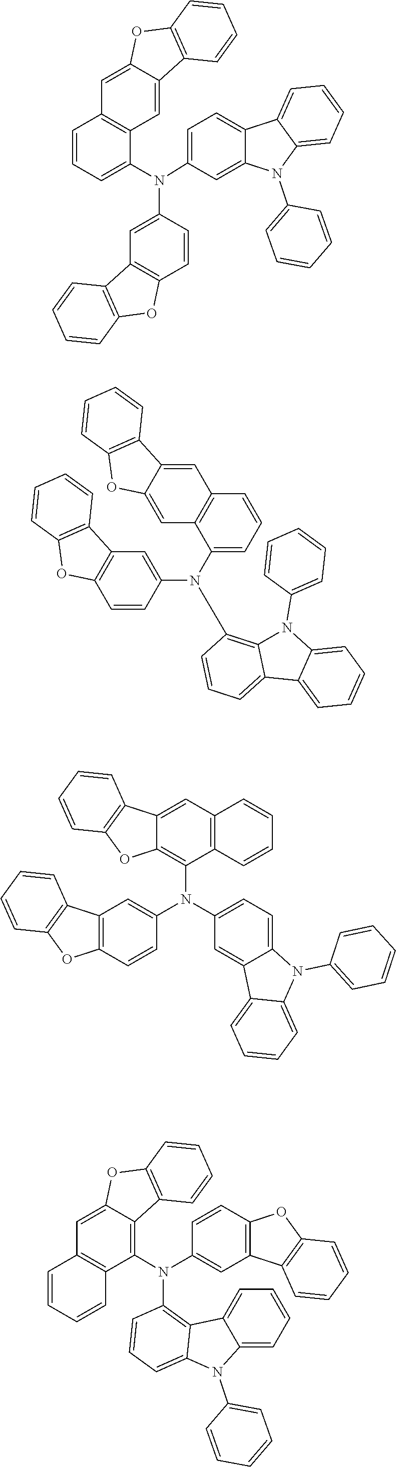

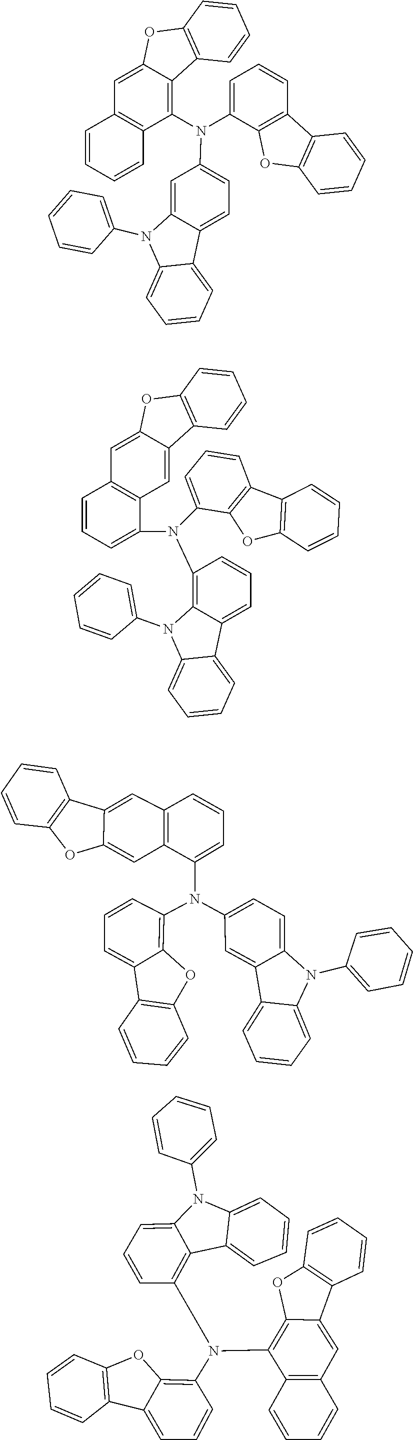

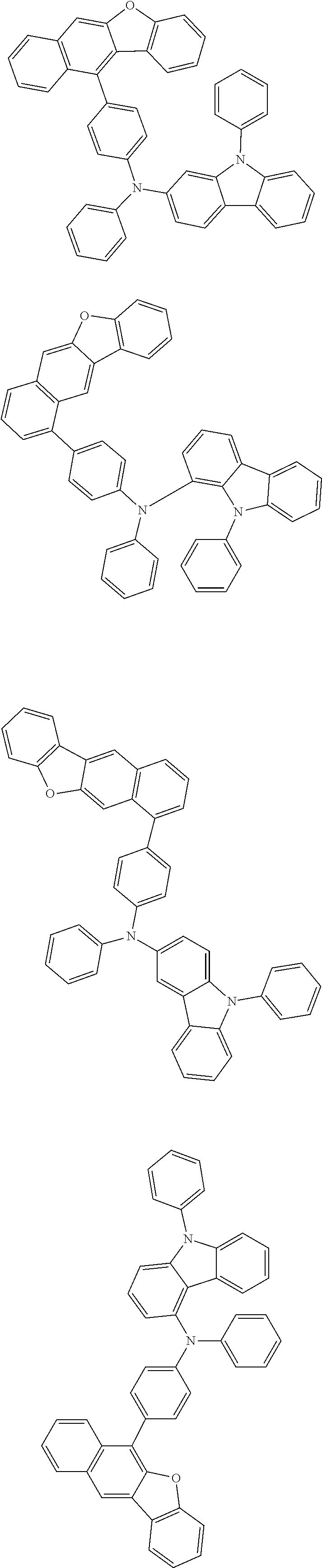 [SMILES: c1ccc(N(c2ccc(-c3c4ccccc4cc4oc5ccccc5c34)cc2)c2ccc3c4ccccc4n(-c4ccccc4)c3c2)cc1 ; c1ccc(N(c2ccc(-c3cccc4cc5oc6ccccc6c5cc34)cc2)c2cccc3c4ccccc4n(-c4ccccc4)c23)cc1 ; c1ccc(N(c2ccc(-c3cccc4cc5c(cc34)oc3ccccc35)cc2)c2ccc3c(c2)c2ccccc2n3-c2ccccc2)cc1 ; c1ccc(N(c2ccc(-c3c4ccccc4cc4c3oc3ccccc34)cc2)c2cccc3c2c2ccccc2n3-c2ccccc2)cc1]

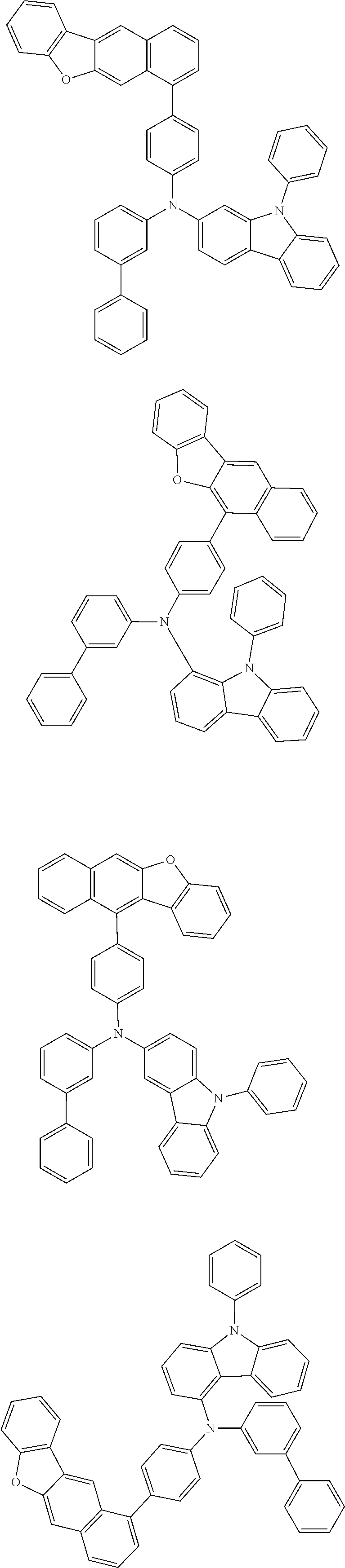 [SMILES: c1ccc(-c2cccc(N(c3ccc(-c4cccc5cc6c(cc45)oc4ccccc46)cc3)c3ccc4c5ccccc5n(-c5ccccc5)c4c3)c2)cc1 ; c1ccc(-c2cccc(N(c3ccc(-c4c5ccccc5cc5c4oc4ccccc45)cc3)c3cccc4c5ccccc5n(-c5ccccc5)c34)c2)cc1 ; c1ccc(-c2cccc(N(c3ccc(-c4c5ccccc5cc5oc6ccccc6c45)cc3)c3ccc4c(c3)c3ccccc3n4-c3ccccc3)c2)cc1 ; c1ccc(-c2cccc(N(c3ccc(-c4cccc5cc6oc7ccccc7c6cc45)cc3)c3cccc4c3c3ccccc3n4-c3ccccc3)c2)cc1]

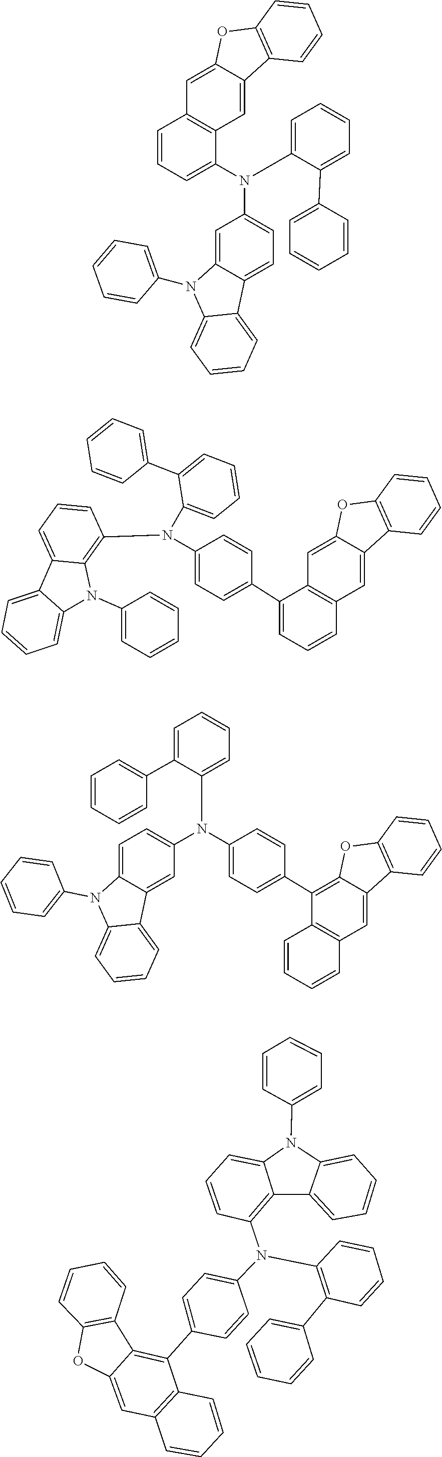 [SMILES: c1ccc(-c2ccccc2N(c2ccc3c4ccccc4n(-c4ccccc4)c3c2)c2cccc3cc4oc5ccccc5c4cc23)cc1 ; c1ccc(-c2ccccc2N(c2ccc(-c3cccc4cc5c(cc34)oc3ccccc35)cc2)c2cccc3c4ccccc4n(-c4ccccc4)c23)cc1 ; c1ccc(-c2ccccc2N(c2ccc(-c3c4ccccc4cc4c3oc3ccccc34)cc2)c2ccc3c(c2)c2ccccc2n3-c2ccccc2)cc1 ; c1ccc(-c2ccccc2N(c2ccc(-c3c4ccccc4cc4oc5ccccc5c34)cc2)c2cccc3c2c2ccccc2n3-c2ccccc2)cc1]

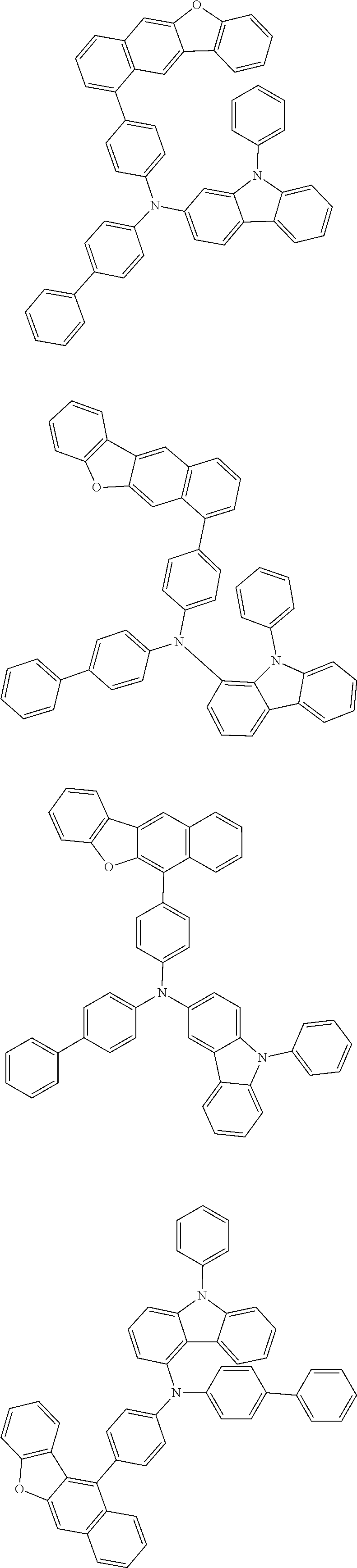 [SMILES: c1ccc(-c2ccc(N(c3ccc(-c4cccc5cc6oc7ccccc7c6cc45)cc3)c3ccc4c5ccccc5n(-c5ccccc5)c4c3)cc2)cc1 ; c1ccc(-c2ccc(N(c3ccc(-c4cccc5cc6c(cc45)oc4ccccc46)cc3)c3cccc4c5ccccc5n(-c5ccccc5)c34)cc2)cc1 ; c1ccc(-c2ccc(N(c3ccc(-c4c5ccccc5cc5c4oc4ccccc45)cc3)c3ccc4c(c3)c3ccccc3n4-c3ccccc3)cc2)cc1 ; c1ccc(-c2ccc(N(c3ccc(-c4c5ccccc5cc5oc6ccccc6c45)cc3)c3cccc4c3c3ccccc3n4-c3ccccc3)cc2)cc1]

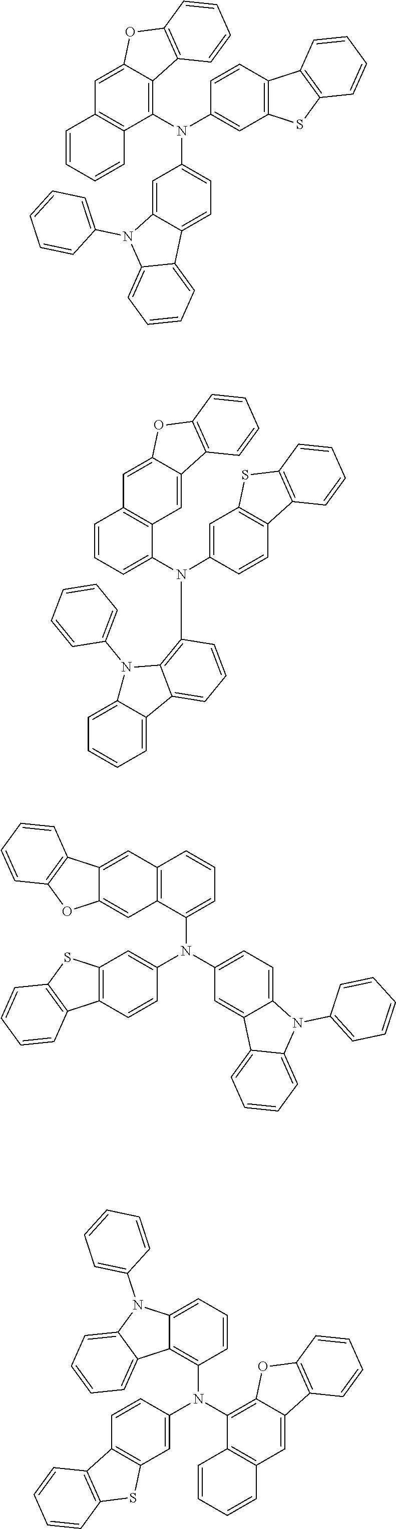 [SMILES: c1ccc(-n2c3ccccc3c3ccc(N(c4ccc5c(c4)sc4ccccc45)c4c5ccccc5cc5oc6ccccc6c45)cc32)cc1 ; c1ccc(-n2c3ccccc3c3cccc(N(c4ccc5c(c4)sc4ccccc45)c4cccc5cc6oc7ccccc7c6cc45)c32)cc1 ; c1ccc(-n2c3ccccc3c3cc(N(c4ccc5c(c4)sc4ccccc45)c4cccc5cc6c(cc45)oc4ccccc46)ccc32)cc1 ; c1ccc(-n2c3ccccc3c3c(N(c4ccc5c(c4)sc4ccccc45)c4c5ccccc5cc5c4oc4ccccc45)cccc32)cc1]

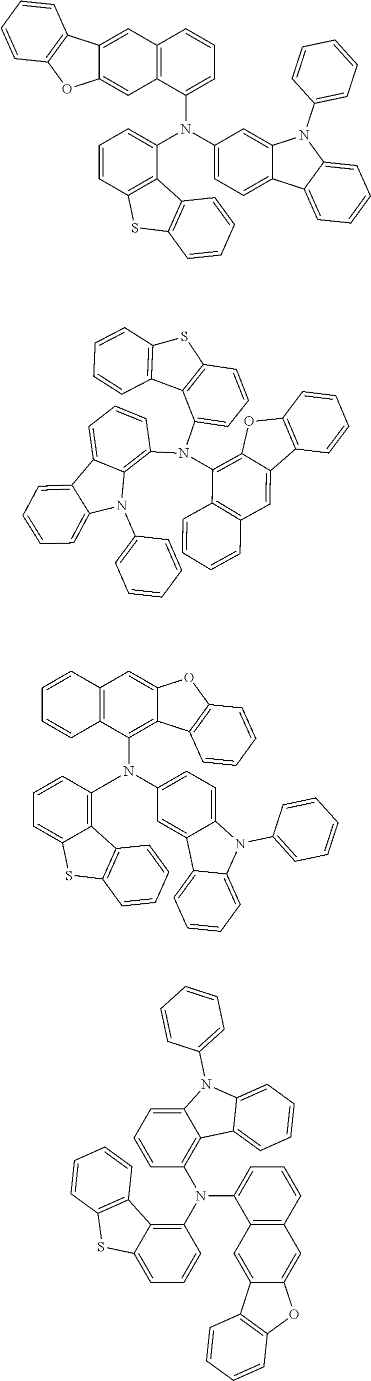 [SMILES: c1ccc(-n2c3ccccc3c3ccc(N(c4cccc5cc6c(cc45)oc4ccccc46)c4cccc5sc6ccccc6c45)cc32)cc1 ; c1ccc(-n2c3ccccc3c3cccc(N(c4c5ccccc5cc5c4oc4ccccc45)c4cccc5sc6ccccc6c45)c32)cc1 ; c1ccc(-n2c3ccccc3c3cc(N(c4c5ccccc5cc5oc6ccccc6c45)c4cccc5sc6ccccc6c45)ccc32)cc1 ; c1ccc(-n2c3ccccc3c3c(N(c4cccc5cc6oc7ccccc7c6cc45)c4cccc5sc6ccccc6c45)cccc32)cc1]

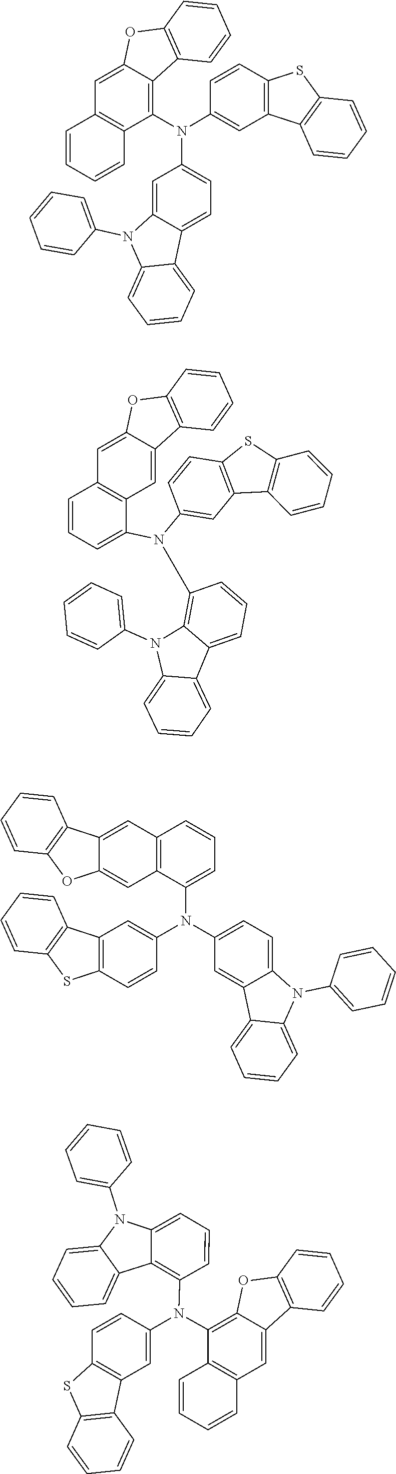 [SMILES: c1ccc(-n2c3ccccc3c3ccc(N(c4ccc5sc6ccccc6c5c4)c4c5ccccc5cc5oc6ccccc6c45)cc32)cc1 ; c1ccc(-n2c3ccccc3c3cccc(N(c4ccc5sc6ccccc6c5c4)c4cccc5cc6oc7ccccc7c6cc45)c32)cc1 ; c1ccc(-n2c3ccccc3c3cc(N(c4ccc5sc6ccccc6c5c4)c4cccc5cc6c(cc45)oc4ccccc46)ccc32)cc1 ; c1ccc(-n2c3ccccc3c3c(N(c4ccc5sc6ccccc6c5c4)c4c5ccccc5cc5c4oc4ccccc45)cccc32)cc1]

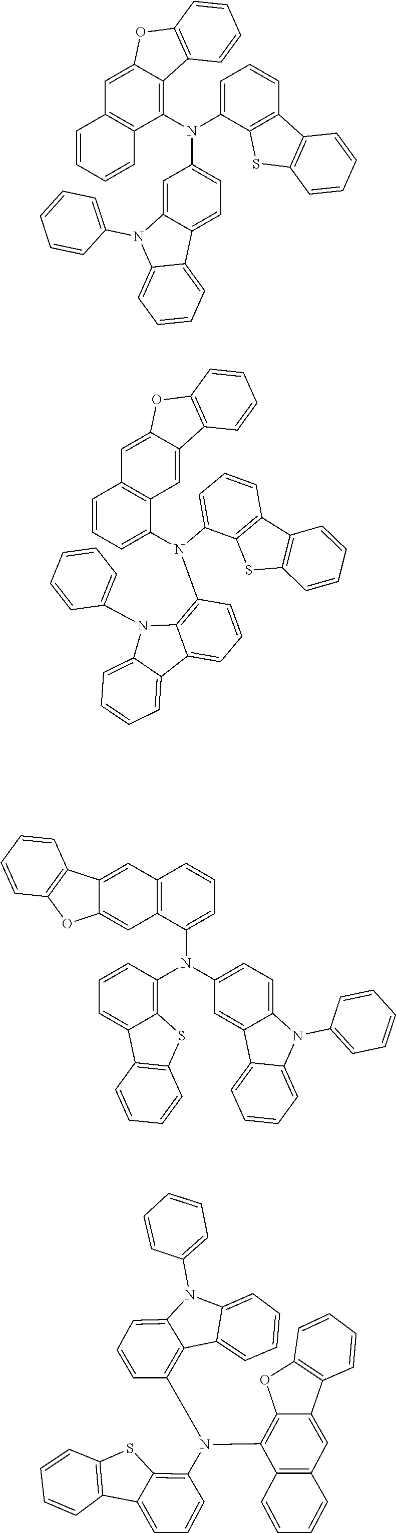 [SMILES: c1ccc(-n2c3ccccc3c3ccc(N(c4cccc5c4sc4ccccc45)c4c5ccccc5cc5oc6ccccc6c45)cc32)cc1 ; c1ccc(-n2c3ccccc3c3cccc(N(c4cccc5cc6oc7ccccc7c6cc45)c4cccc5c4sc4ccccc45)c32)cc1 ; c1ccc(-n2c3ccccc3c3cc(N(c4cccc5cc6c(cc45)oc4ccccc46)c4cccc5c4sc4ccccc45)ccc32)cc1 ; c1ccc(-n2c3ccccc3c3c(N(c4c5ccccc5cc5c4oc4ccccc45)c4cccc5c4sc4ccccc45)cccc32)cc1]

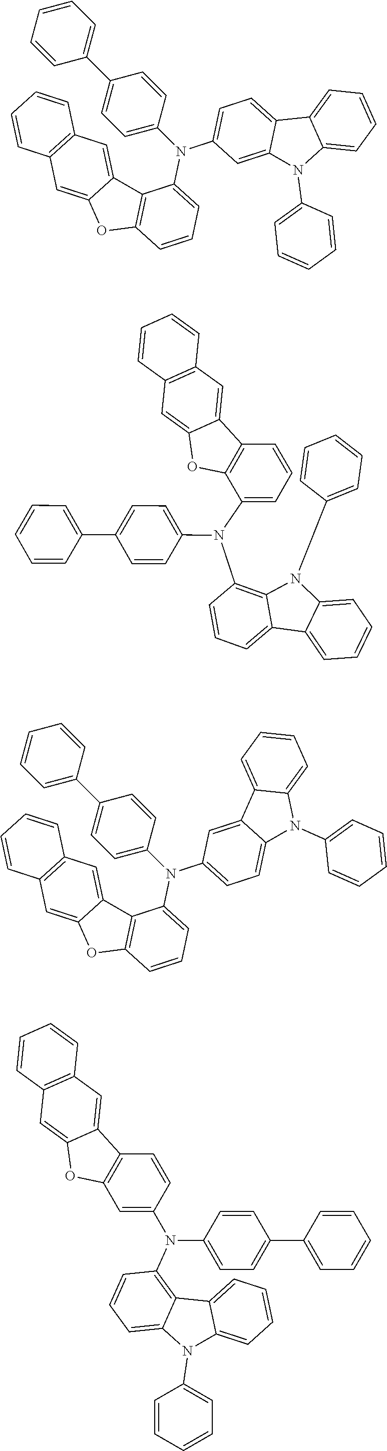 [SMILES: c1ccc(-c2ccc(N(c3ccc4c5ccccc5n(-c5ccccc5)c4c3)c3cccc4oc5cc6ccccc6cc5c34)cc2)cc1 ; c1ccc(-c2ccc(N(c3cccc4c3oc3cc5ccccc5cc34)c3cccc4c5ccccc5n(-c5ccccc5)c34)cc2)cc1 ; c1ccc(-c2ccc(N(c3ccc4c(c3)c3ccccc3n4-c3ccccc3)c3cccc4oc5cc6ccccc6cc5c34)cc2)cc1 ; c1ccc(-c2ccc(N(c3ccc4c(c3)oc3cc5ccccc5cc34)c3cccc4c3c3ccccc3n4-c3ccccc3)cc2)cc1]

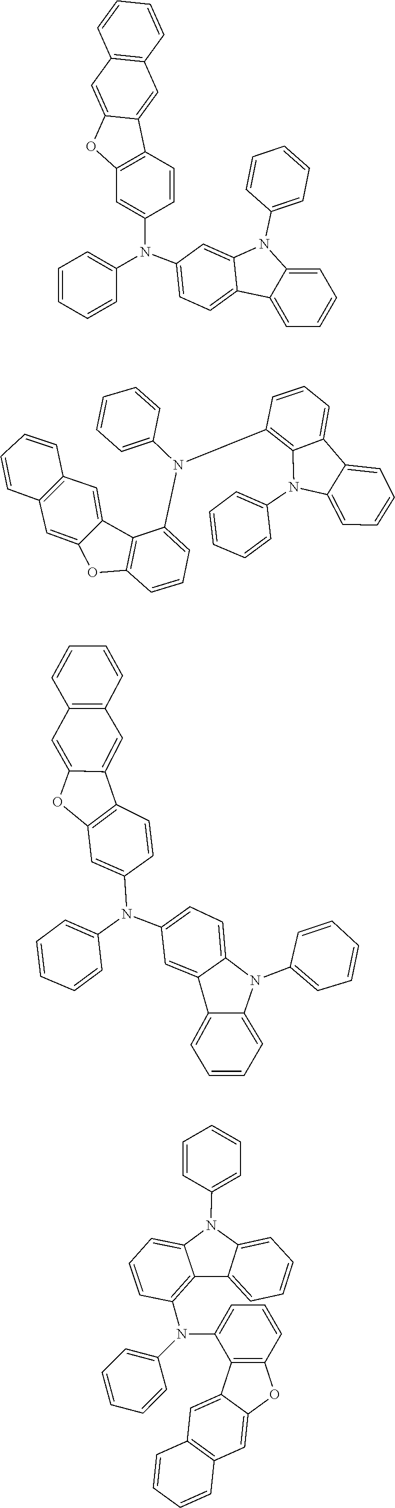 [SMILES: c1ccc(N(c2ccc3c(c2)oc2cc4ccccc4cc23)c2ccc3c4ccccc4n(-c4ccccc4)c3c2)cc1 ; c1ccc(N(c2cccc3oc4cc5ccccc5cc4c23)c2cccc3c4ccccc4n(-c4ccccc4)c23)cc1 ; c1ccc(N(c2ccc3c(c2)oc2cc4ccccc4cc23)c2ccc3c(c2)c2ccccc2n3-c2ccccc2)cc1 ; c1ccc(N(c2cccc3oc4cc5ccccc5cc4c23)c2cccc3c2c2ccccc2n3-c2ccccc2)cc1]

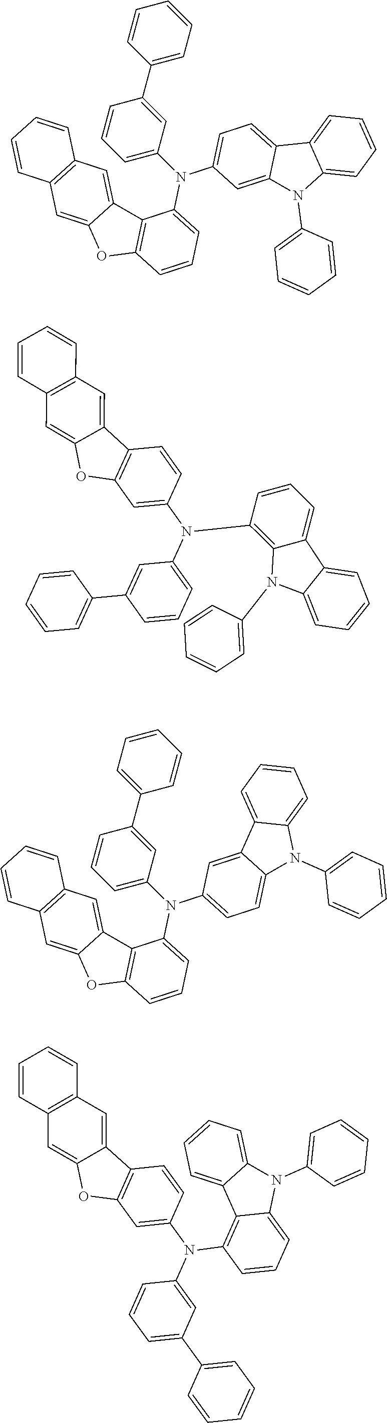 [SMILES: c1ccc(-c2cccc(N(c3ccc4c5ccccc5n(-c5ccccc5)c4c3)c3cccc4oc5cc6ccccc6cc5c34)c2)cc1 ; c1ccc(-c2cccc(N(c3ccc4c(c3)oc3cc5ccccc5cc34)c3cccc4c5ccccc5n(-c5ccccc5)c34)c2)cc1 ; c1ccc(-c2cccc(N(c3ccc4c(c3)c3ccccc3n4-c3ccccc3)c3cccc4oc5cc6ccccc6cc5c34)c2)cc1 ; c1ccc(-c2cccc(N(c3ccc4c(c3)oc3cc5ccccc5cc34)c3cccc4c3c3ccccc3n4-c3ccccc3)c2)cc1]

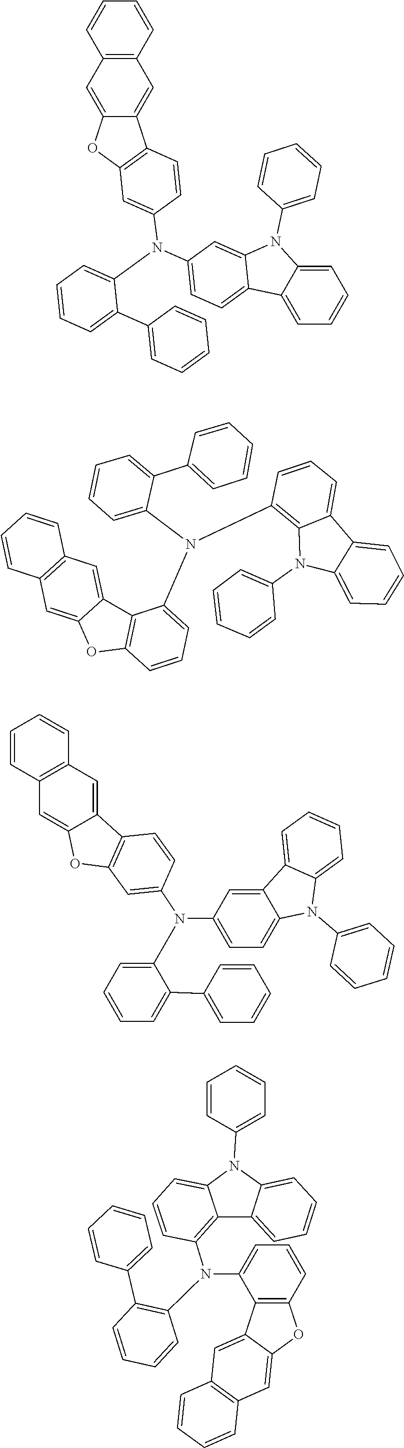

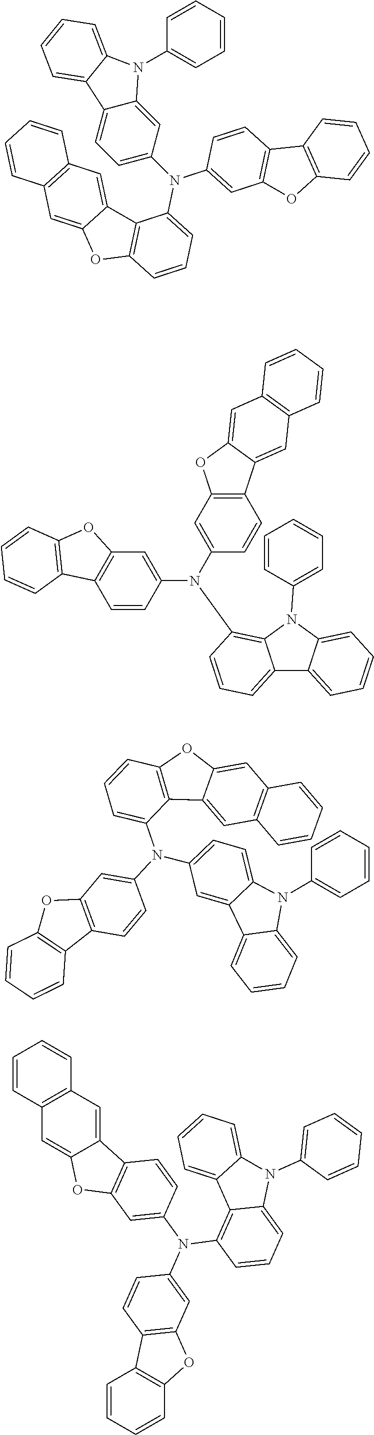

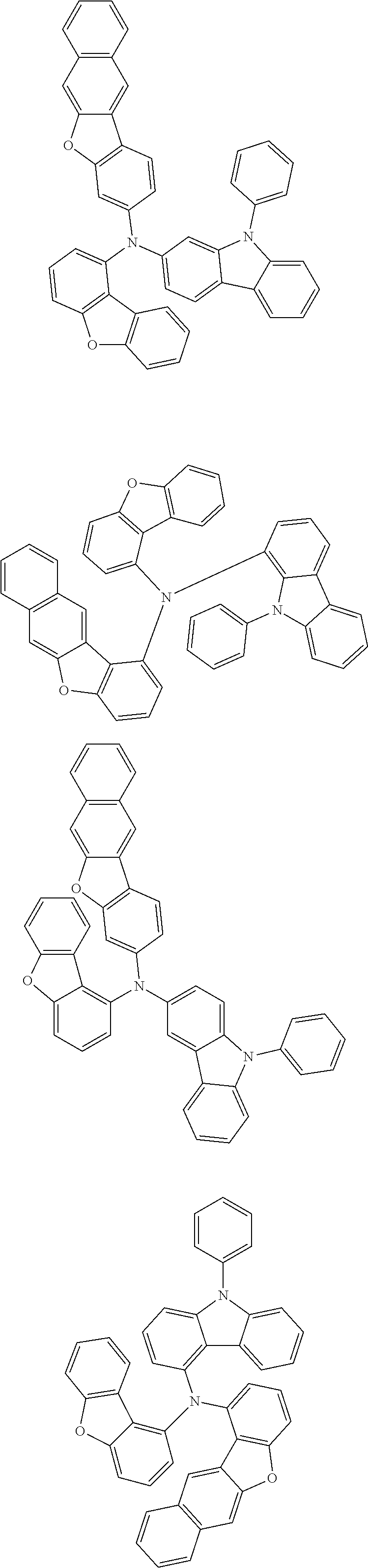

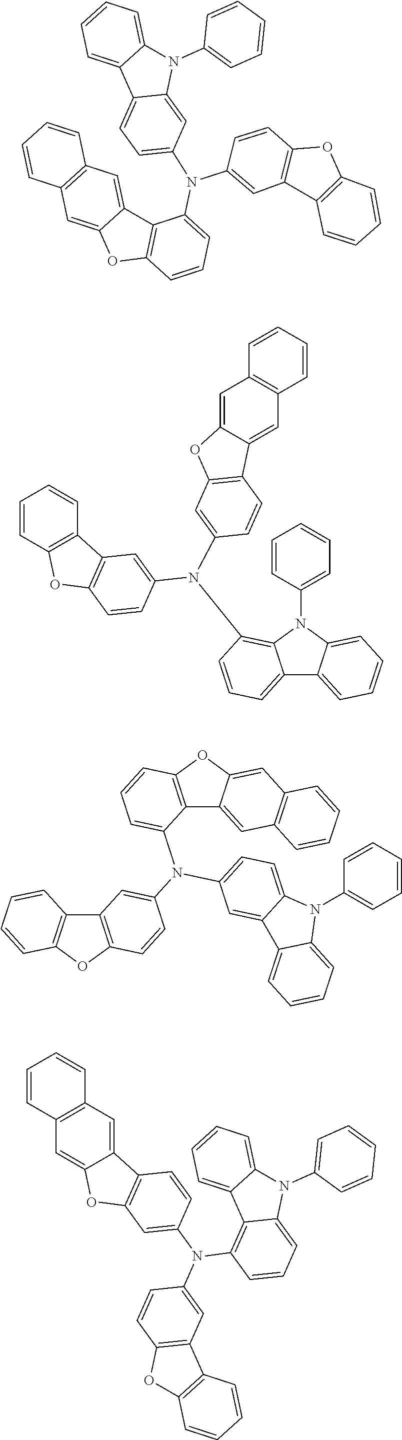 [SMILES: c1ccc(-n2c3ccccc3c3ccc(N(c4ccc5oc6ccccc6c5c4)c4cccc5oc6cc7ccccc7cc6c45)cc32)cc1 ; c1ccc(-n2c3ccccc3c3cccc(N(c4ccc5c(c4)oc4cc6ccccc6cc45)c4ccc5oc6ccccc6c5c4)c32)cc1 ; c1ccc(-n2c3ccccc3c3cc(N(c4ccc5oc6ccccc6c5c4)c4cccc5oc6cc7ccccc7cc6c45)ccc32)cc1 ; c1ccc(-n2c3ccccc3c3c(N(c4ccc5c(c4)oc4cc6ccccc6cc45)c4ccc5oc6ccccc6c5c4)cccc32)cc1]

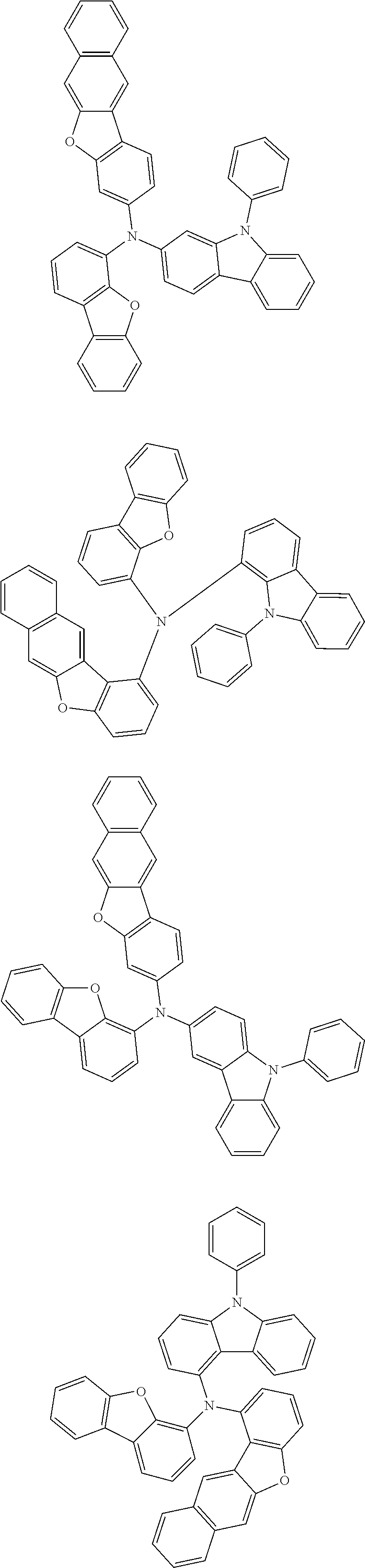

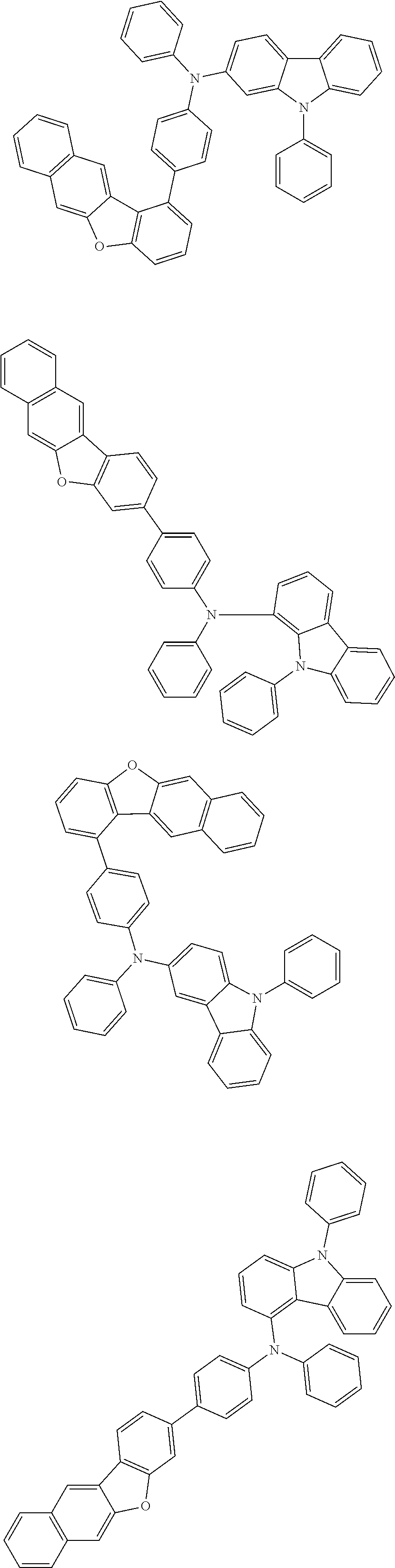 [SMILES: c1ccc(N(c2ccc(-c3cccc4oc5cc6ccccc6cc5c34)cc2)c2ccc3c4ccccc4n(-c4ccccc4)c3c2)cc1 ; c1ccc(N(c2ccc(-c3ccc4c(c3)oc3cc5ccccc5cc34)cc2)c2cccc3c4ccccc4n(-c4ccccc4)c23)cc1 ; c1ccc(N(c2ccc(-c3cccc4oc5cc6ccccc6cc5c34)cc2)c2ccc3c(c2)c2ccccc2n3-c2ccccc2)cc1 ; c1ccc(N(c2ccc(-c3ccc4c(c3)oc3cc5ccccc5cc34)cc2)c2cccc3c2c2ccccc2n3-c2ccccc2)cc1]

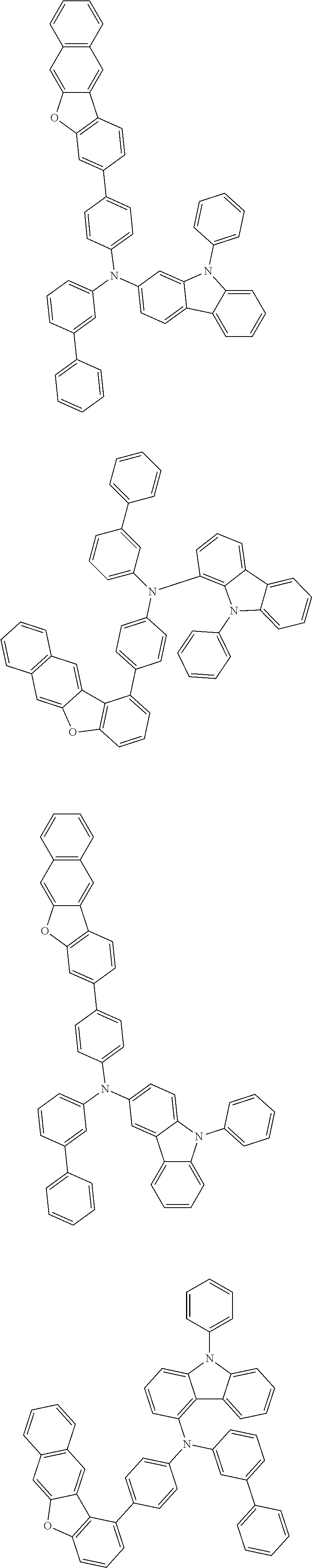 [SMILES: c1ccc(-c2cccc(N(c3ccc(-c4ccc5c(c4)oc4cc6ccccc6cc45)cc3)c3ccc4c5ccccc5n(-c5ccccc5)c4c3)c2)cc1 ; c1ccc(-c2cccc(N(c3ccc(-c4cccc5oc6cc7ccccc7cc6c45)cc3)c3cccc4c5ccccc5n(-c5ccccc5)c34)c2)cc1 ; c1ccc(-c2cccc(N(c3ccc(-c4ccc5c(c4)oc4cc6ccccc6cc45)cc3)c3ccc4c(c3)c3ccccc3n4-c3ccccc3)c2)cc1 ; c1ccc(-c2cccc(N(c3ccc(-c4cccc5oc6cc7ccccc7cc6c45)cc3)c3cccc4c3c3ccccc3n4-c3ccccc3)c2)cc1]

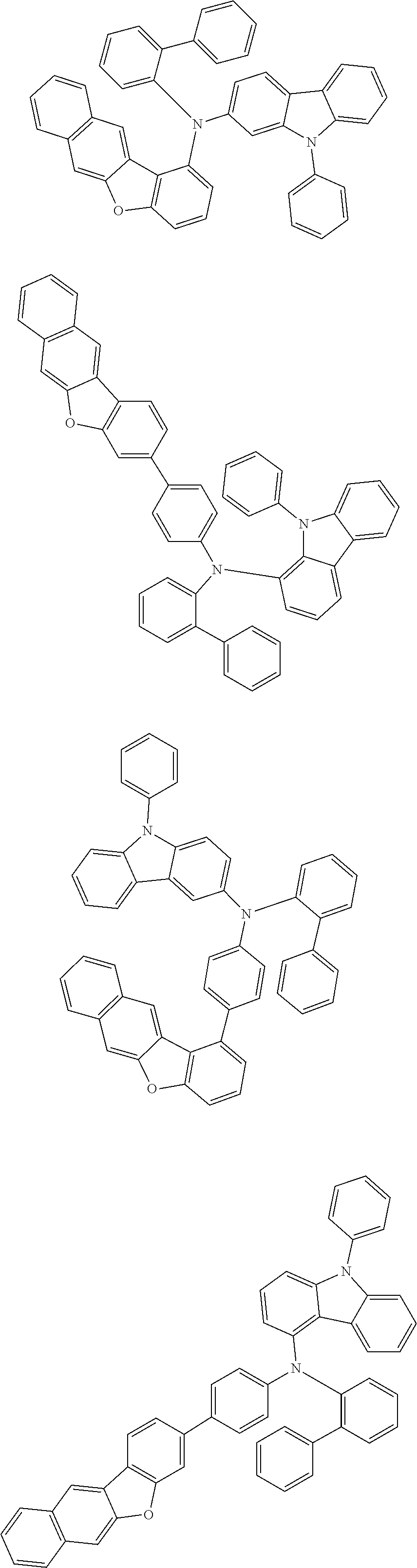 [SMILES: c1ccc(-c2ccccc2N(c2ccc3c4ccccc4n(-c4ccccc4)c3c2)c2cccc3oc4cc5ccccc5cc4c23)cc1 ; c1ccc(-c2ccccc2N(c2ccc(-c3ccc4c(c3)oc3cc5ccccc5cc34)cc2)c2cccc3c4ccccc4n(-c4ccccc4)c23)cc1 ; c1ccc(-c2ccccc2N(c2ccc(-c3cccc4oc5cc6ccccc6cc5c34)cc2)c2ccc3c(c2)c2ccccc2n3-c2ccccc2)cc1 ; c1ccc(-c2ccccc2N(c2ccc(-c3ccc4c(c3)oc3cc5ccccc5cc34)cc2)c2cccc3c2c2ccccc2n3-c2ccccc2)cc1]

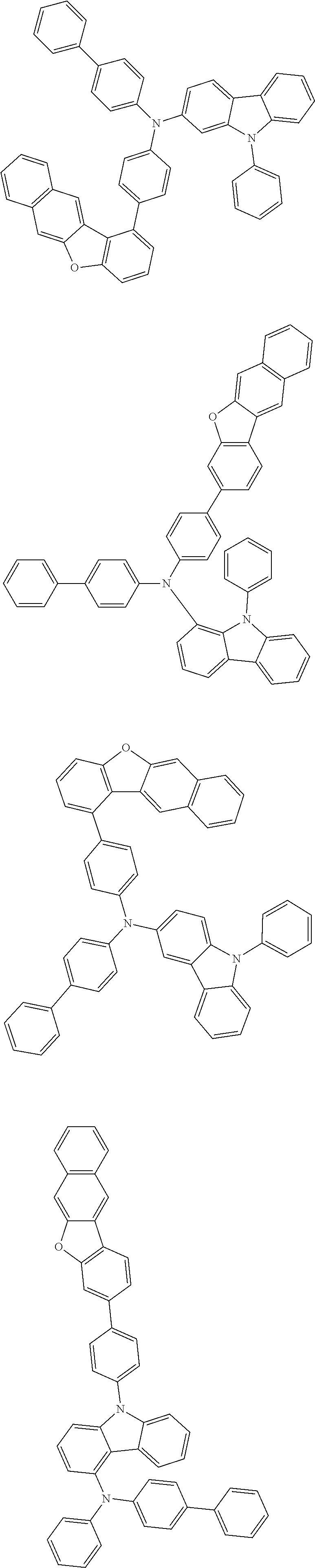 [SMILES: c1ccc(-c2ccc(N(c3ccc(-c4cccc5oc6cc7ccccc7cc6c45)cc3)c3ccc4c5ccccc5n(-c5ccccc5)c4c3)cc2)cc1 ; c1ccc(-c2ccc(N(c3ccc(-c4ccc5c(c4)oc4cc6ccccc6cc45)cc3)c3cccc4c5ccccc5n(-c5ccccc5)c34)cc2)cc1 ; c1ccc(-c2ccc(N(c3ccc(-c4cccc5oc6cc7ccccc7cc6c45)cc3)c3ccc4c(c3)c3ccccc3n4-c3ccccc3)cc2)cc1 ; c1ccc(-c2ccc(N(c3ccccc3)c3cccc4c3c3ccccc3n4-c3ccc(-c4ccc5c(c4)oc4cc6ccccc6cc45)cc3)cc2)cc1]

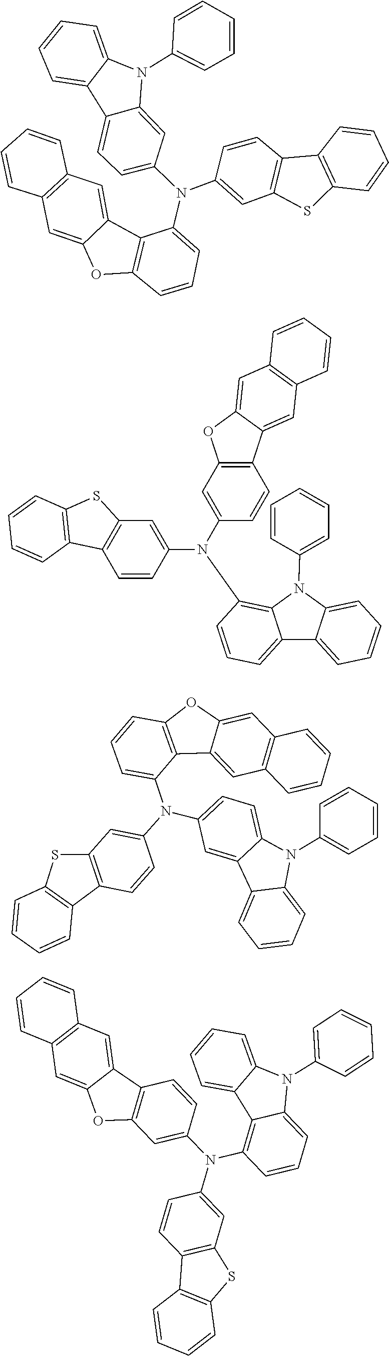

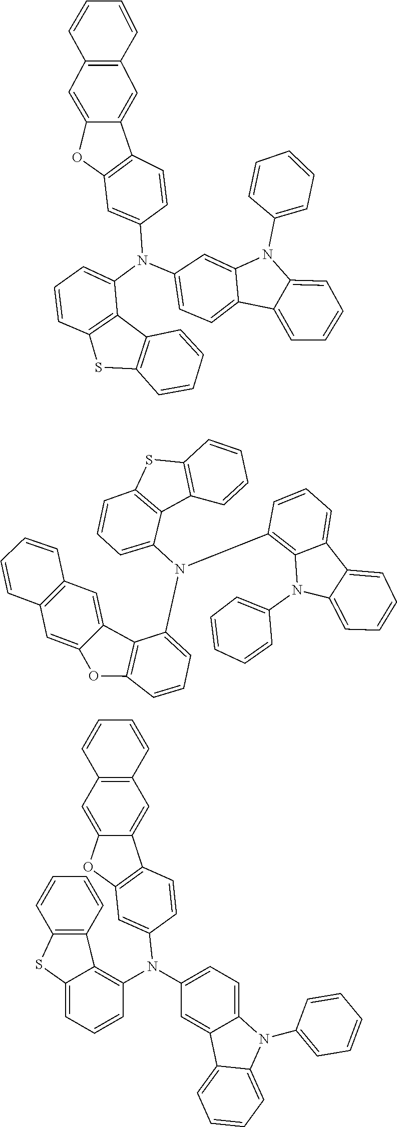

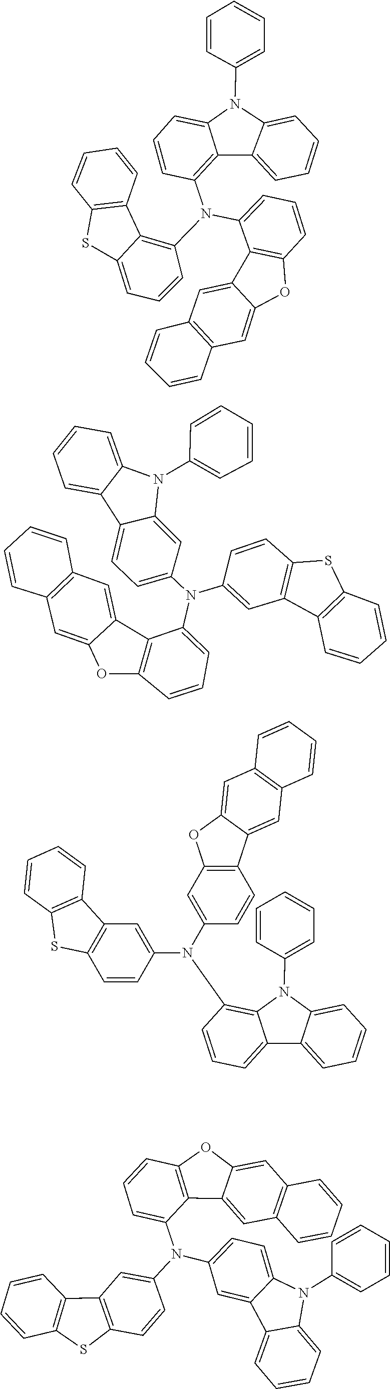

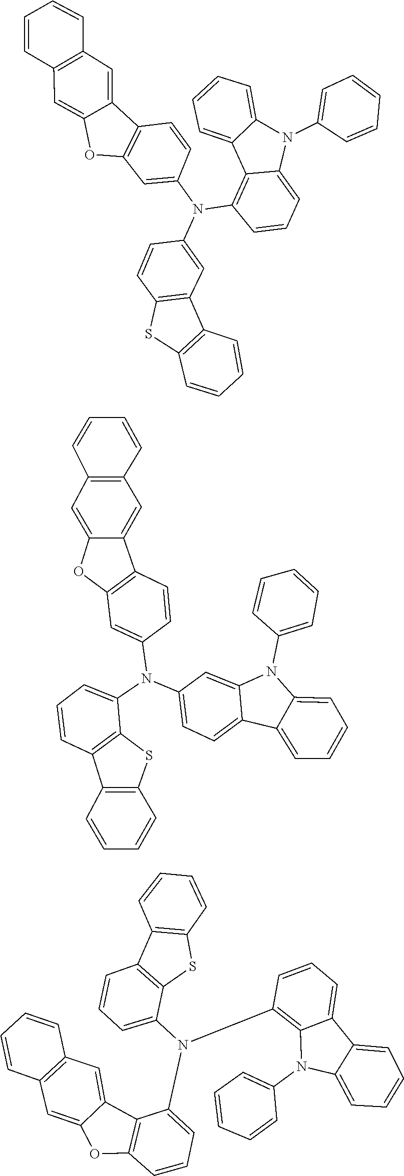

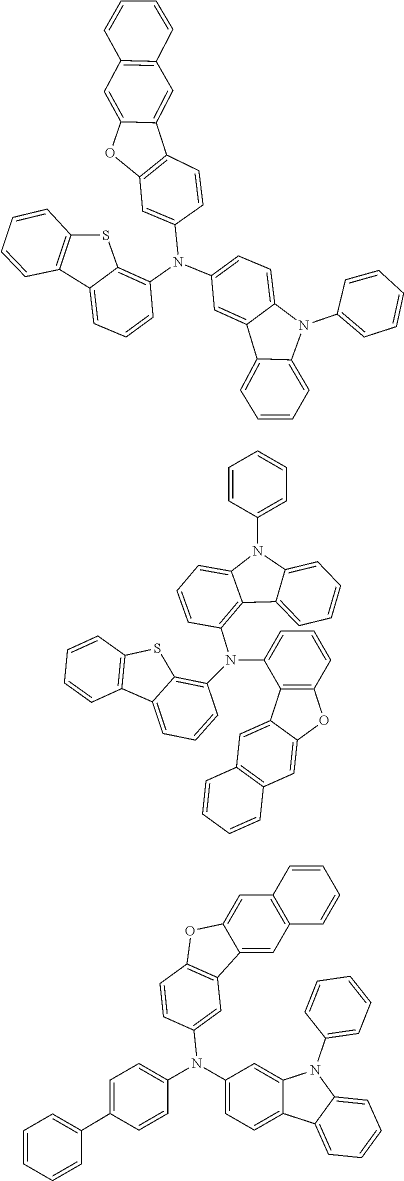

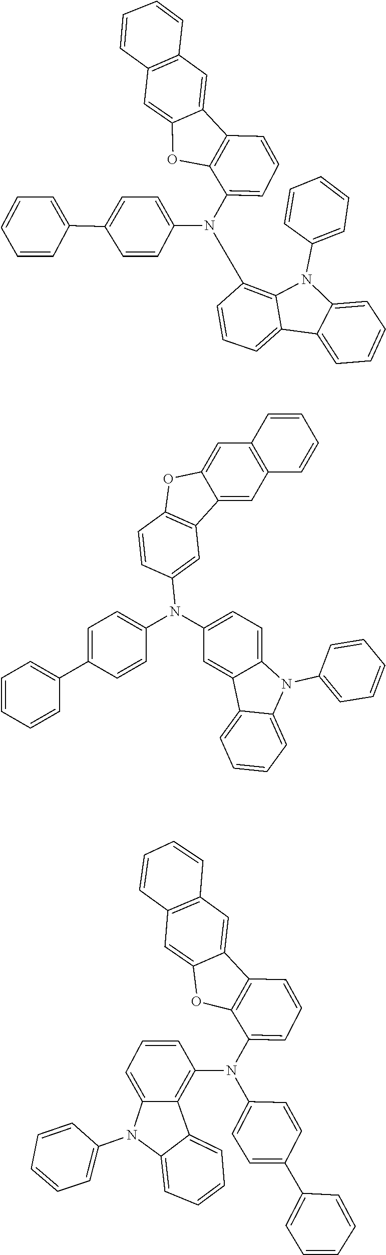 [SMILES: c1ccc(-c2ccc(N(c3cccc4c3oc3cc5ccccc5cc34)c3cccc4c5ccccc5n(-c5ccccc5)c34)cc2)cc1 ; c1ccc(-c2ccc(N(c3ccc4oc5cc6ccccc6cc5c4c3)c3ccc4c(c3)c3ccccc3n4-c3ccccc3)cc2)cc1 ; c1ccc(-c2ccc(N(c3cccc4c3oc3cc5ccccc5cc34)c3cccc4c3c3ccccc3n4-c3ccccc3)cc2)cc1]

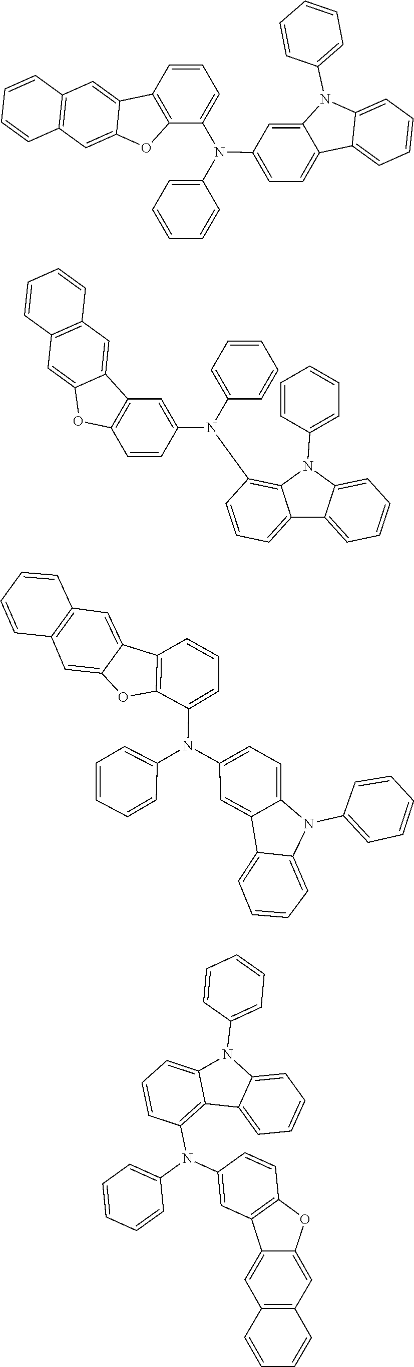

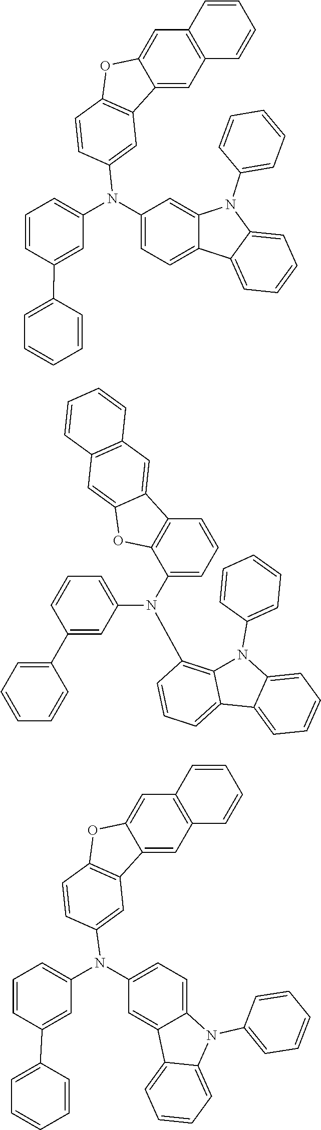

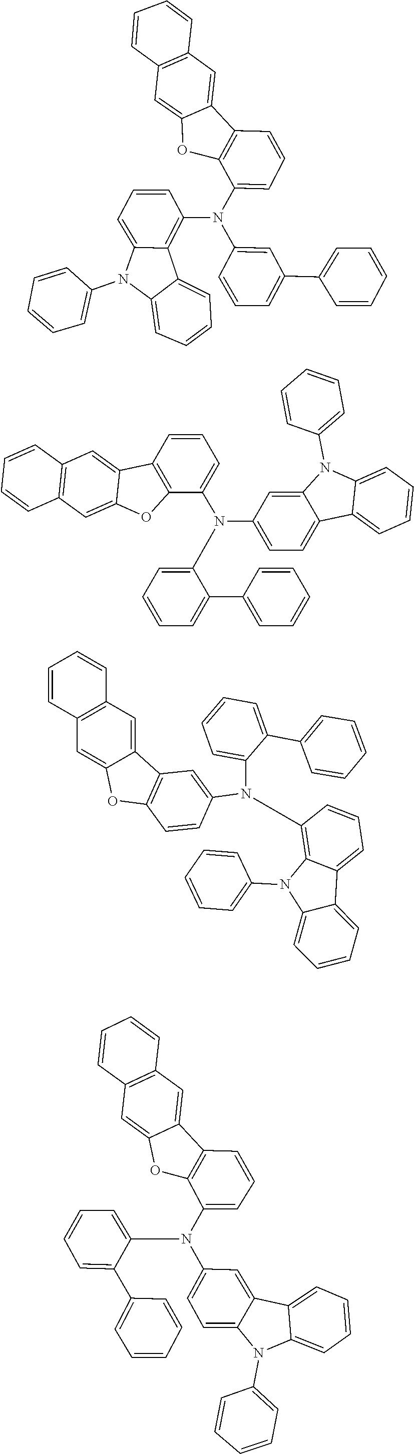 [SMILES: c1ccc(-c2cccc(N(c3cccc4c3oc3cc5ccccc5cc34)c3cccc4c3c3ccccc3n4-c3ccccc3)c2)cc1 ; c1ccc(-c2ccccc2N(c2ccc3c4ccccc4n(-c4ccccc4)c3c2)c2cccc3c2oc2cc4ccccc4cc23)cc1 ; c1ccc(-c2ccccc2N(c2ccc3oc4cc5ccccc5cc4c3c2)c2cccc3c4ccccc4n(-c4ccccc4)c23)cc1 ; c1ccc(-c2ccccc2N(c2ccc3c(c2)c2ccccc2n3-c2ccccc2)c2cccc3c2oc2cc4ccccc4cc23)cc1]

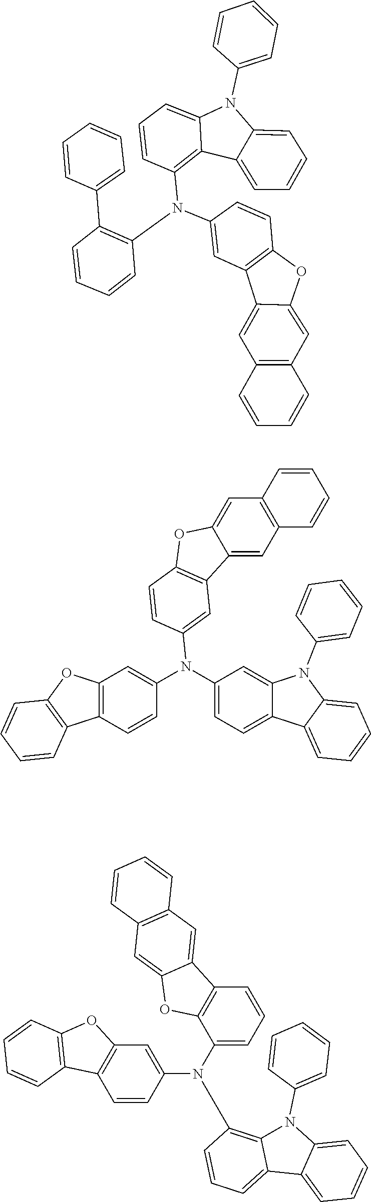

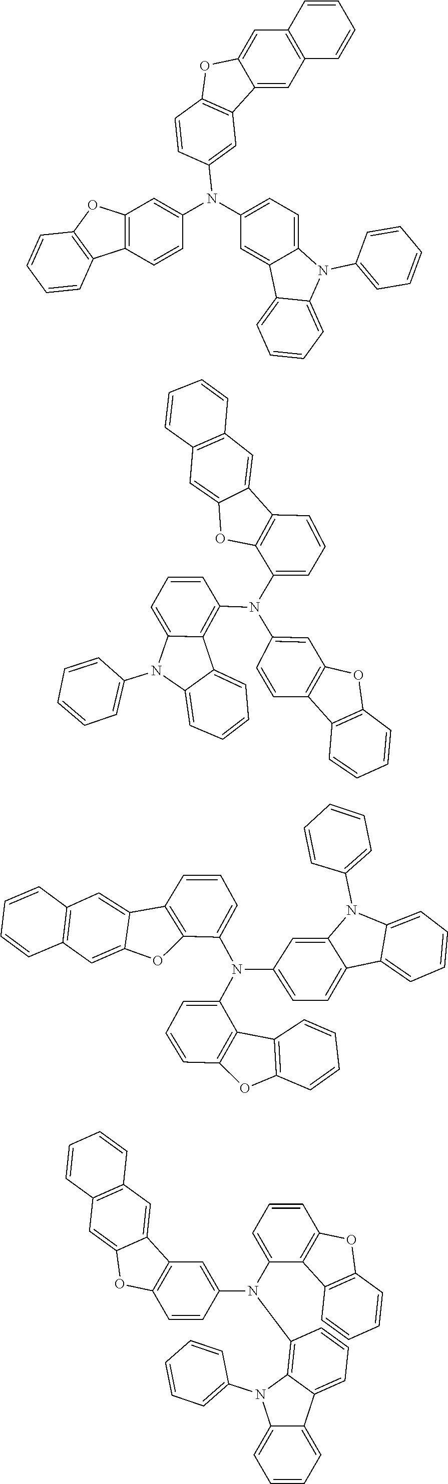

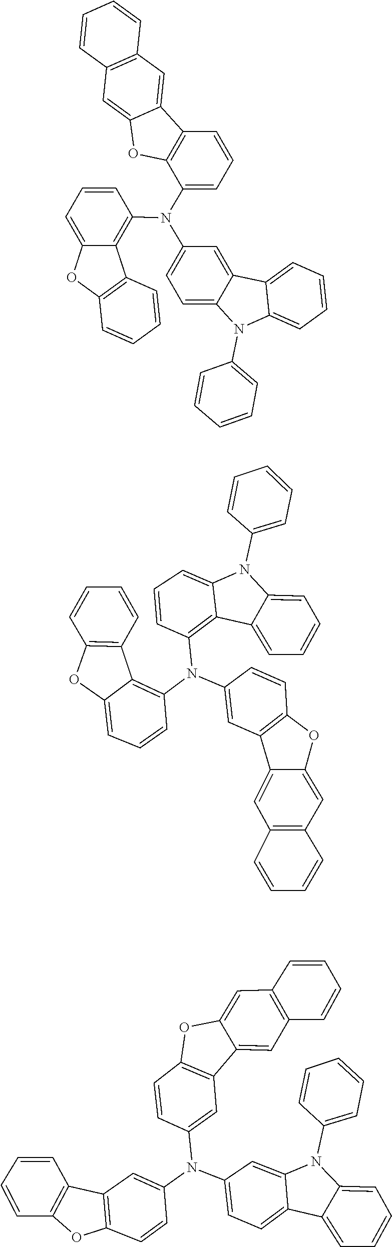 [SMILES: c1ccc(-n2c3ccccc3c3cc(N(c4cccc5c4oc4cc6ccccc6cc45)c4cccc5oc6ccccc6c45)ccc32)cc1 ; c1ccc(-n2c3ccccc3c3c(N(c4ccc5oc6cc7ccccc7cc6c5c4)c4cccc5oc6ccccc6c45)cccc32)cc1 ; c1ccc(-n2c3ccccc3c3ccc(N(c4ccc5oc6ccccc6c5c4)c4ccc5oc6cc7ccccc7cc6c5c4)cc32)cc1]

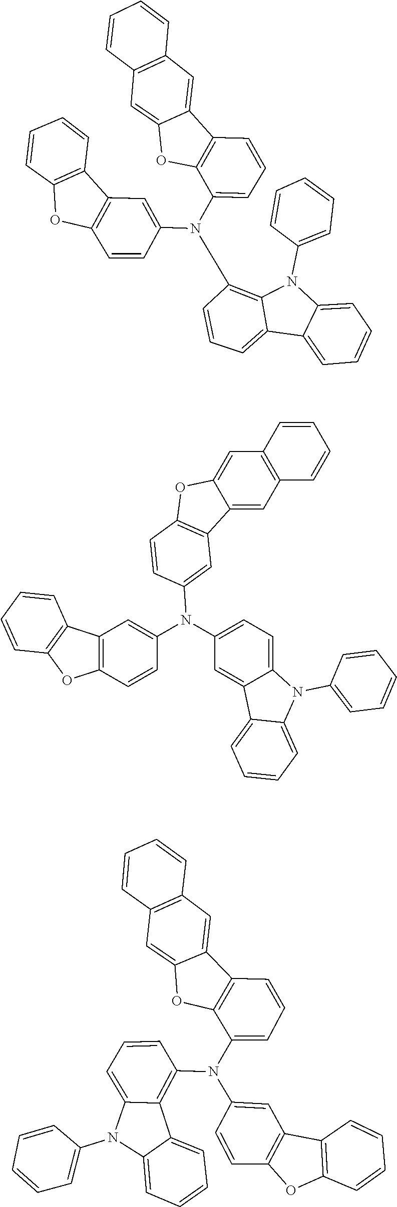 [SMILES: c1ccc(-n2c3ccccc3c3cccc(N(c4ccc5oc6ccccc6c5c4)c4cccc5c4oc4cc6ccccc6cc45)c32)cc1 ; c1ccc(-n2c3ccccc3c3cc(N(c4ccc5oc6ccccc6c5c4)c4ccc5oc6cc7ccccc7cc6c5c4)ccc32)cc1 ; c1ccc(-n2c3ccccc3c3c(N(c4ccc5oc6ccccc6c5c4)c4cccc5c4oc4cc6ccccc6cc45)cccc32)cc1]

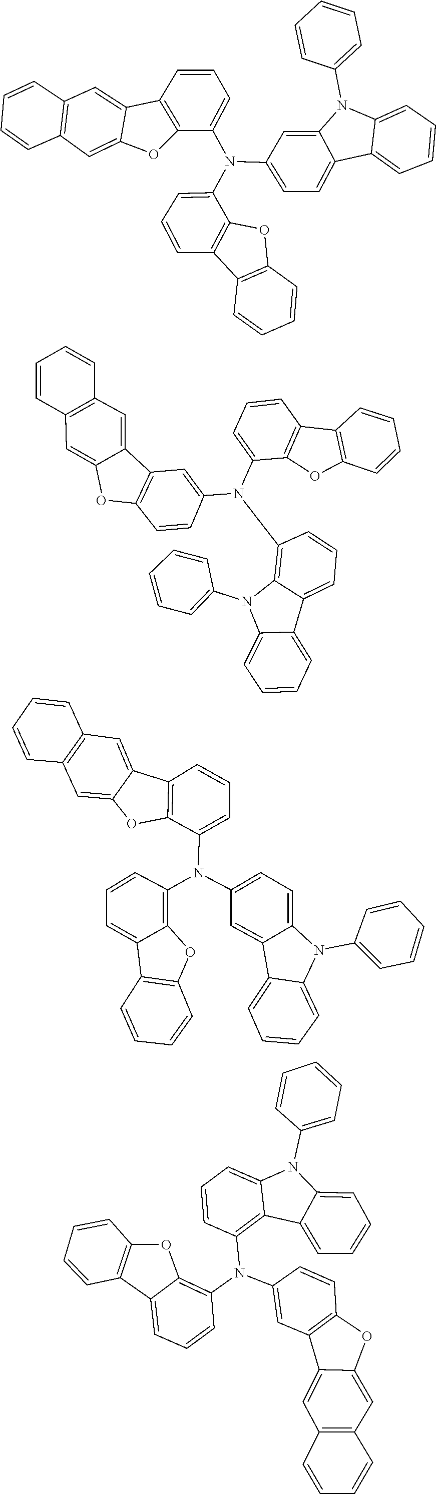

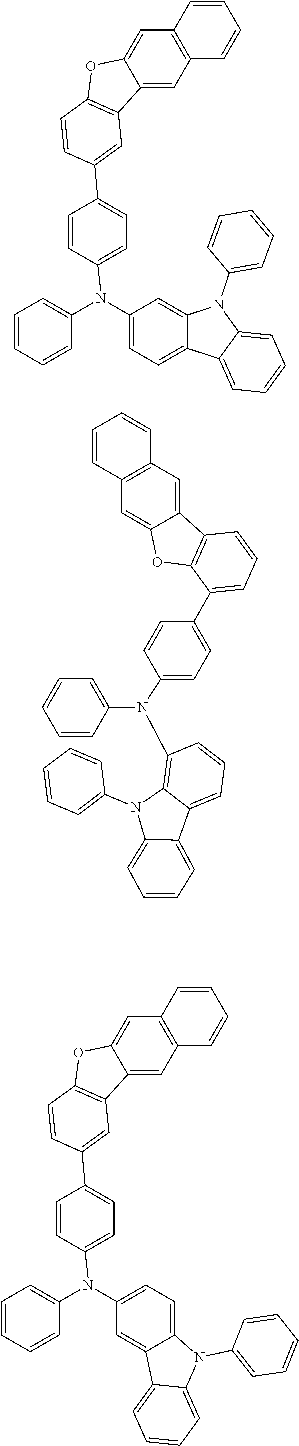 [SMILES: c1ccc(N(c2ccc(-c3ccc4oc5cc6ccccc6cc5c4c3)cc2)c2ccc3c4ccccc4n(-c4ccccc4)c3c2)cc1 ; c1ccc(N(c2ccc(-c3cccc4c3oc3cc5ccccc5cc34)cc2)c2cccc3c4ccccc4n(-c4ccccc4)c23)cc1 ; c1ccc(N(c2ccc(-c3ccc4oc5cc6ccccc6cc5c4c3)cc2)c2ccc3c(c2)c2ccccc2n3-c2ccccc2)cc1]

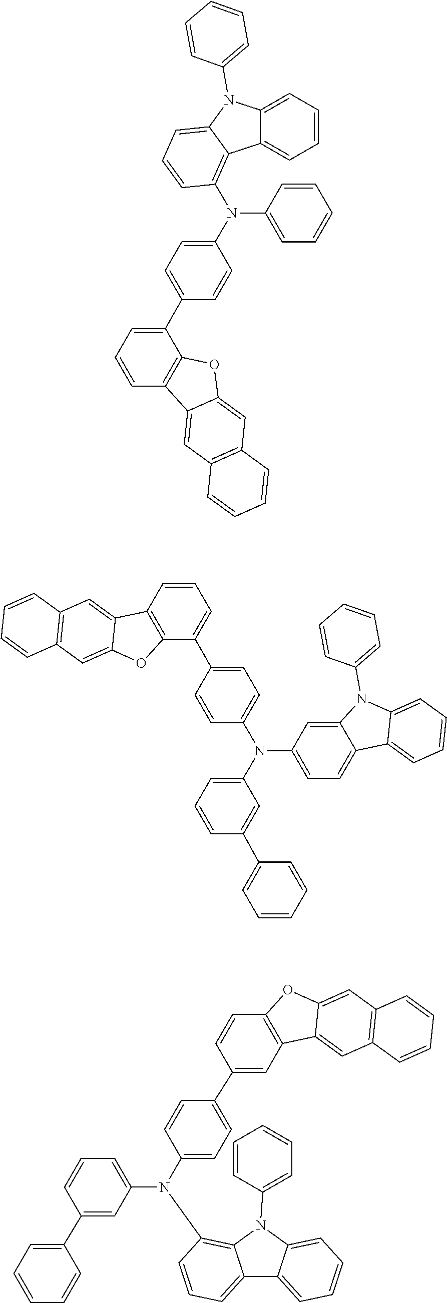 [SMILES: c1ccc(N(c2ccc(-c3cccc4c3oc3cc5ccccc5cc34)cc2)c2cccc3c2c2ccccc2n3-c2ccccc2)cc1 ; c1ccc(-c2cccc(N(c3ccc(-c4cccc5c4oc4cc6ccccc6cc45)cc3)c3ccc4c5ccccc5n(-c5ccccc5)c4c3)c2)cc1 ; c1ccc(-c2cccc(N(c3ccc(-c4ccc5oc6cc7ccccc7cc6c5c4)cc3)c3cccc4c5ccccc5n(-c5ccccc5)c34)c2)cc1]

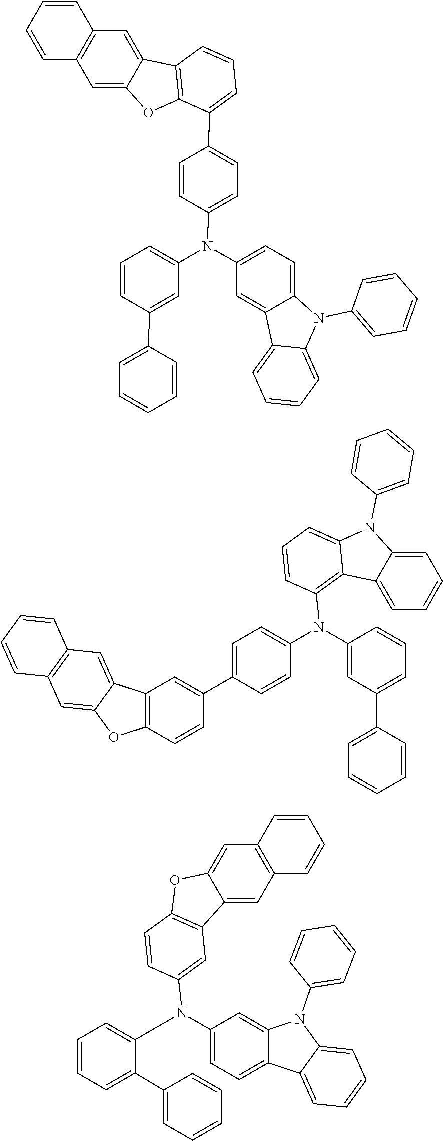 [SMILES: c1ccc(-c2cccc(N(c3ccc(-c4cccc5c4oc4cc6ccccc6cc45)cc3)c3ccc4c(c3)c3ccccc3n4-c3ccccc3)c2)cc1 ; c1ccc(-c2cccc(N(c3ccc(-c4ccc5oc6cc7ccccc7cc6c5c4)cc3)c3cccc4c3c3ccccc3n4-c3ccccc3)c2)cc1 ; c1ccc(-c2ccccc2N(c2ccc3oc4cc5ccccc5cc4c3c2)c2ccc3c4ccccc4n(-c4ccccc4)c3c2)cc1]

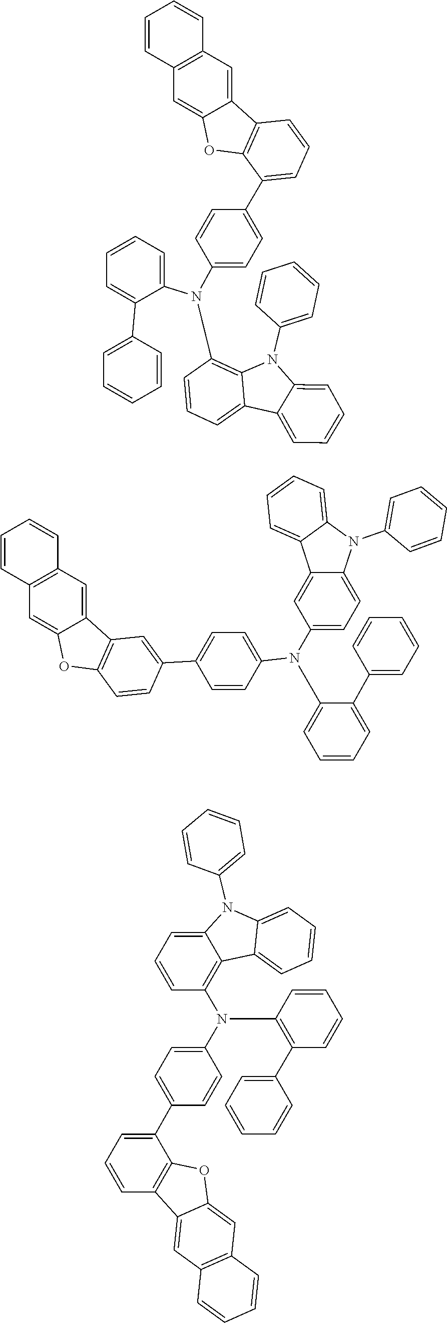

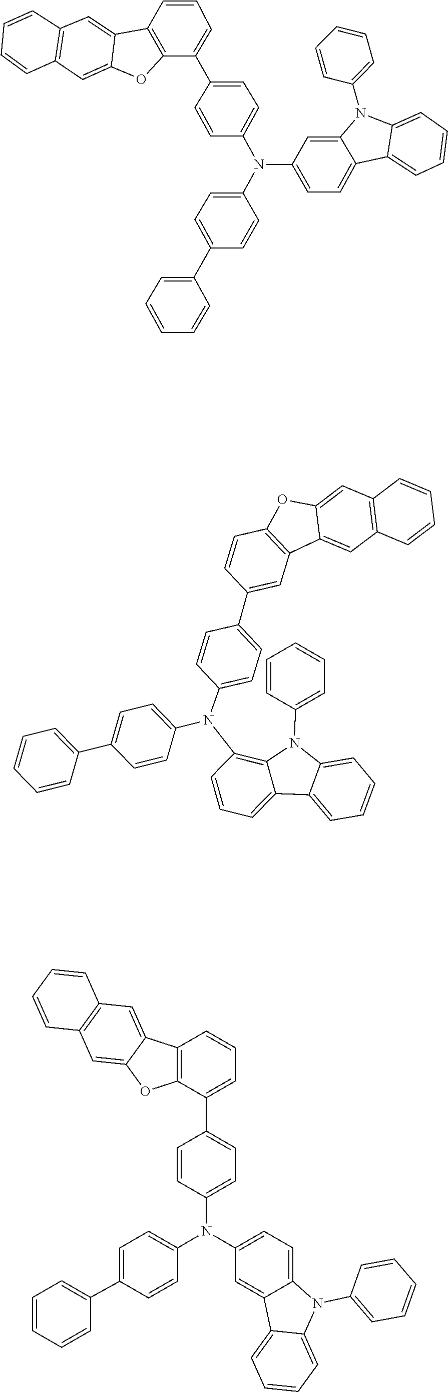 [SMILES: c1ccc(-c2ccc(N(c3ccc(-c4cccc5c4oc4cc6ccccc6cc45)cc3)c3ccc4c5ccccc5n(-c5ccccc5)c4c3)cc2)cc1 ; c1ccc(-c2ccc(N(c3ccc(-c4ccc5oc6cc7ccccc7cc6c5c4)cc3)c3cccc4c5ccccc5n(-c5ccccc5)c34)cc2)cc1 ; c1ccc(-c2ccc(N(c3ccc(-c4cccc5c4oc4cc6ccccc6cc45)cc3)c3ccc4c(c3)c3ccccc3n4-c3ccccc3)cc2)cc1]

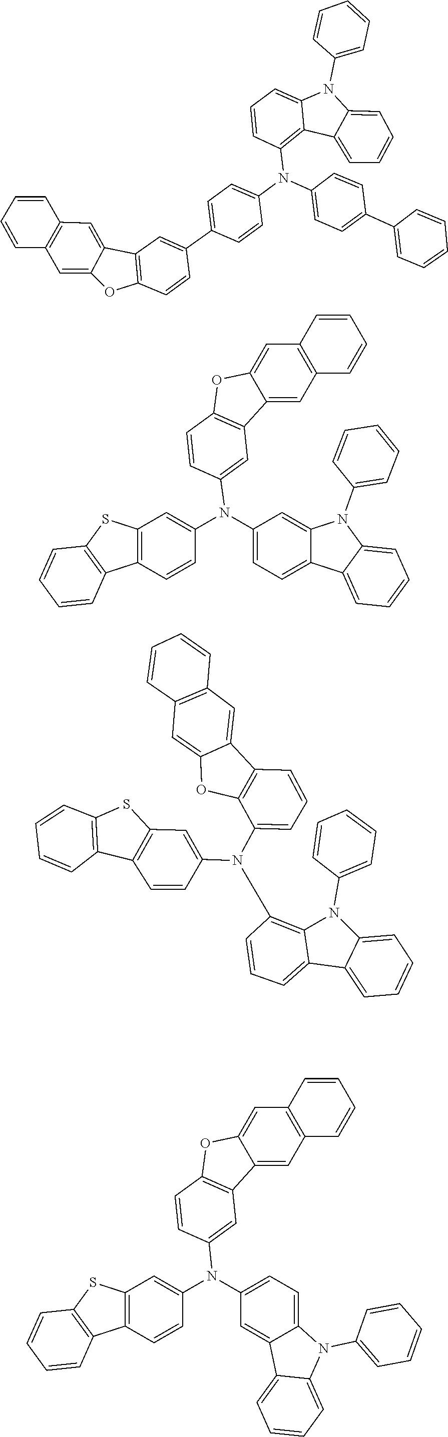 [SMILES: c1ccc(-c2ccc(N(c3ccc(-c4ccc5oc6cc7ccccc7cc6c5c4)cc3)c3cccc4c3c3ccccc3n4-c3ccccc3)cc2)cc1 ; c1ccc(-n2c3ccccc3c3ccc(N(c4ccc5c(c4)sc4ccccc45)c4ccc5oc6cc7ccccc7cc6c5c4)cc32)cc1 ; c1ccc(-n2c3ccccc3c3cccc(N(c4ccc5c(c4)sc4ccccc45)c4cccc5c4oc4cc6ccccc6cc45)c32)cc1 ; c1ccc(-n2c3ccccc3c3cc(N(c4ccc5c(c4)sc4ccccc45)c4ccc5oc6cc7ccccc7cc6c5c4)ccc32)cc1]

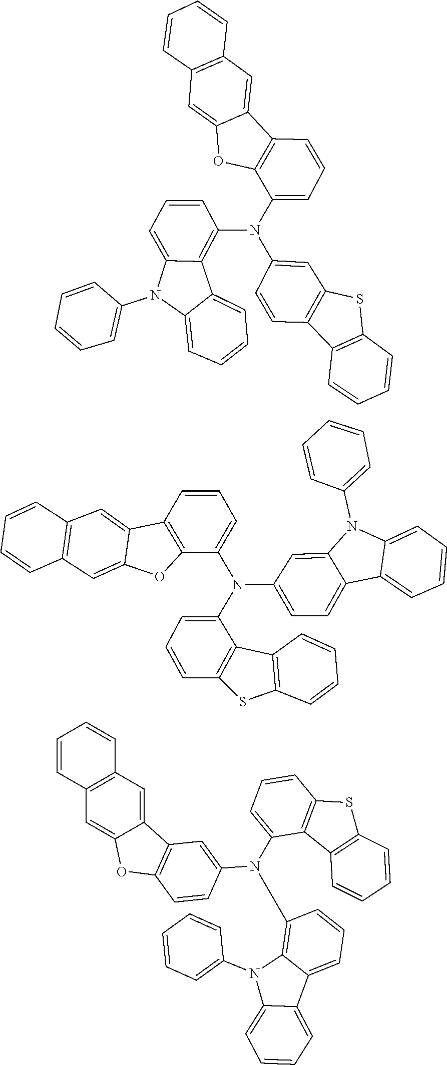

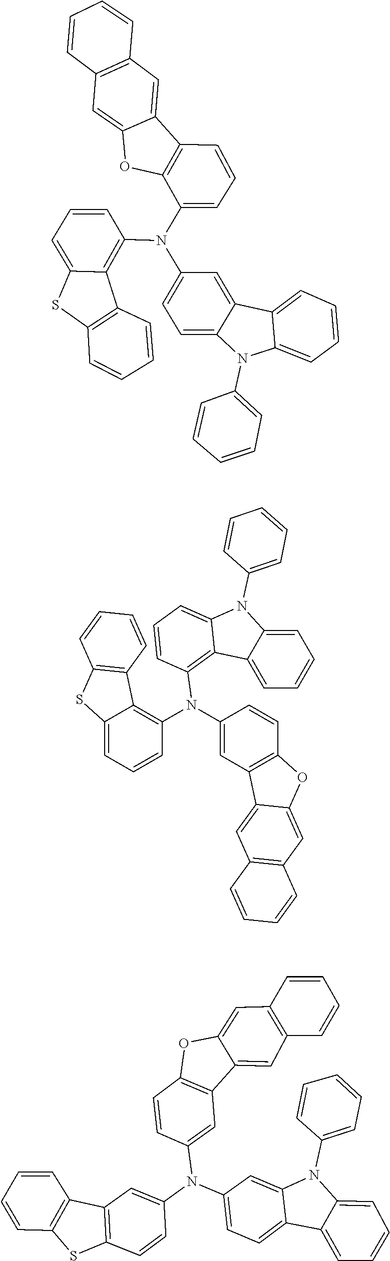 [SMILES: c1ccc(-n2c3ccccc3c3cc(N(c4cccc5c4oc4cc6ccccc6cc45)c4cccc5sc6ccccc6c45)ccc32)cc1 ; c1ccc(-n2c3ccccc3c3c(N(c4ccc5oc6cc7ccccc7cc6c5c4)c4cccc5sc6ccccc6c45)cccc32)cc1 ; c1ccc(-n2c3ccccc3c3ccc(N(c4ccc5oc6cc7ccccc7cc6c5c4)c4ccc5sc6ccccc6c5c4)cc32)cc1]

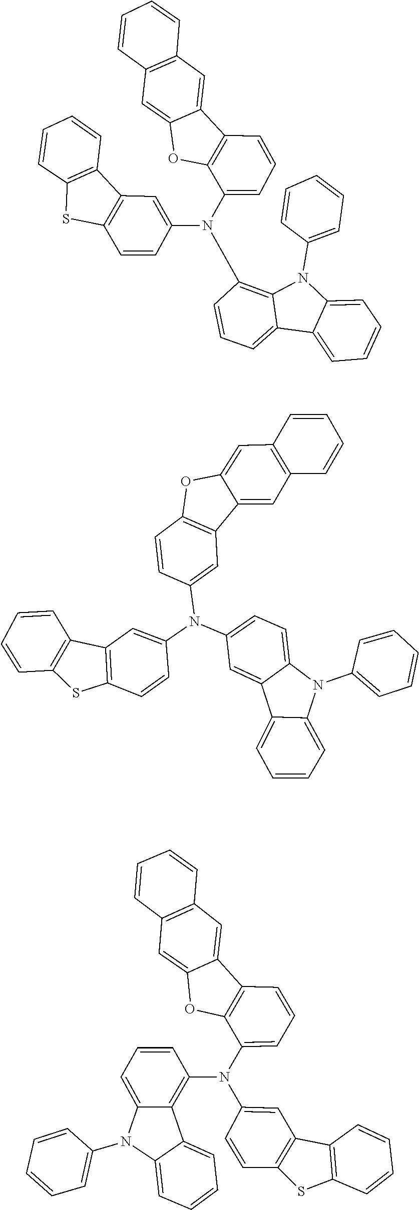 [SMILES: c1ccc(-n2c3ccccc3c3cccc(N(c4ccc5sc6ccccc6c5c4)c4cccc5c4oc4cc6ccccc6cc45)c32)cc1 ; c1ccc(-n2c3ccccc3c3cc(N(c4ccc5oc6cc7ccccc7cc6c5c4)c4ccc5sc6ccccc6c5c4)ccc32)cc1 ; c1ccc(-n2c3ccccc3c3c(N(c4ccc5sc6ccccc6c5c4)c4cccc5c4oc4cc6ccccc6cc45)cccc32)cc1]

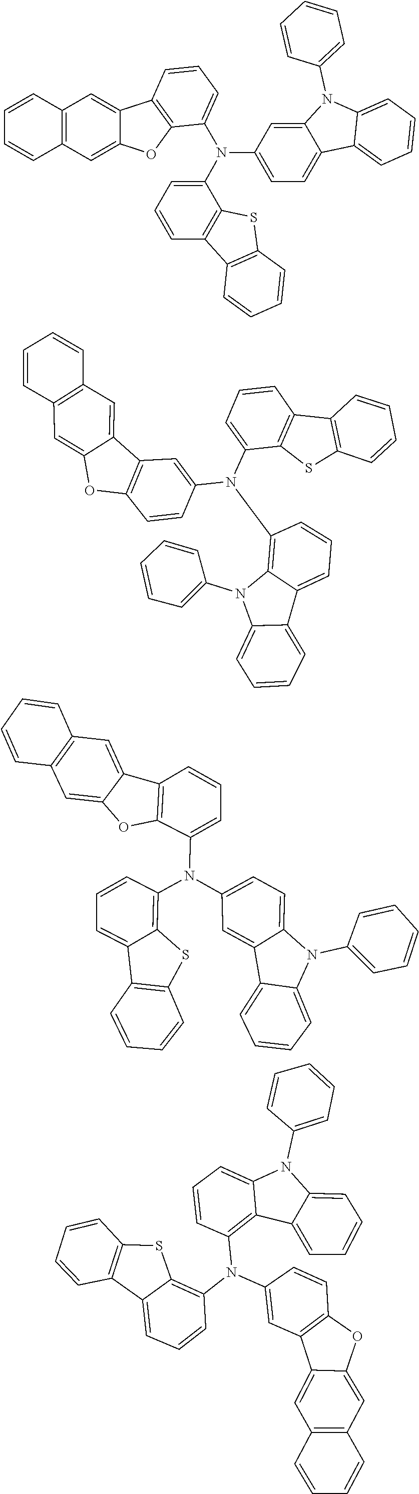

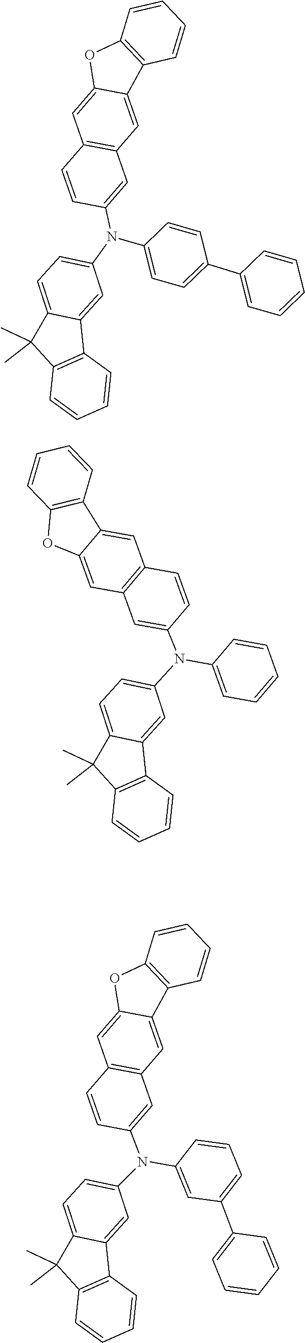

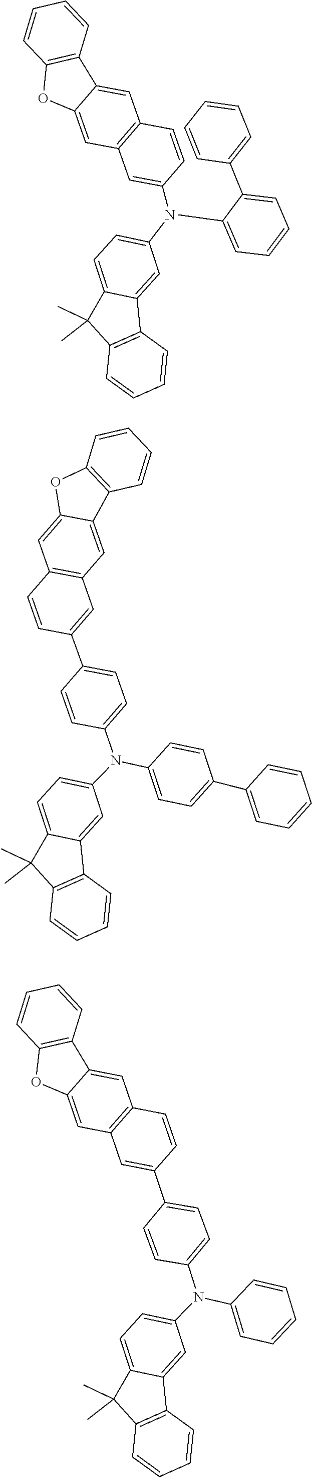

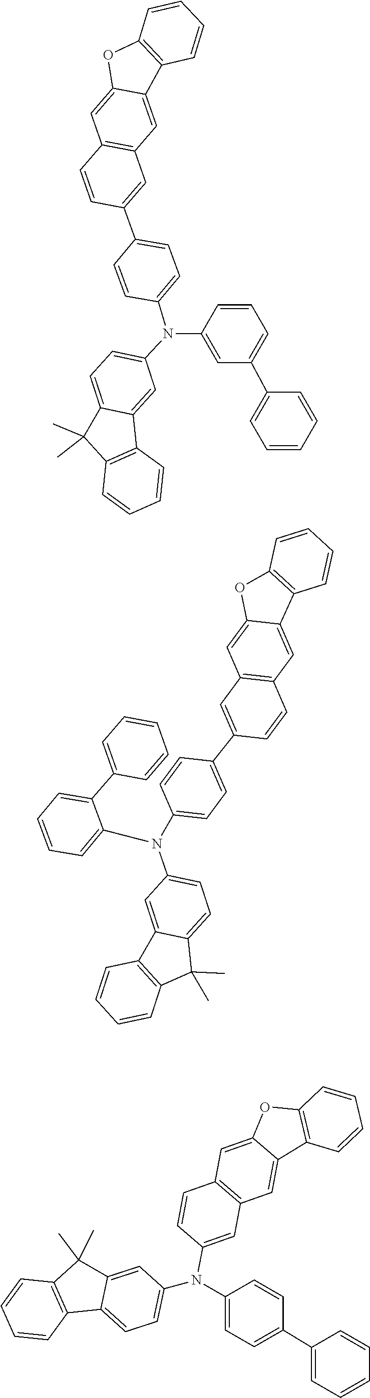 [SMILES: CC1(C)c2ccccc2-c2cc(N(c3ccc(-c4ccc5cc6oc7ccccc7c6cc5c4)cc3)c3cccc(-c4ccccc4)c3)ccc21 ; CC1(C)c2ccccc2-c2cc(N(c3ccc(-c4ccc5cc6c(cc5c4)oc4ccccc46)cc3)c3ccccc3-c3ccccc3)ccc21 ; CC1(C)c2ccccc2-c2ccc(N(c3ccc(-c4ccccc4)cc3)c3ccc4cc5oc6ccccc6c5cc4c3)cc21]

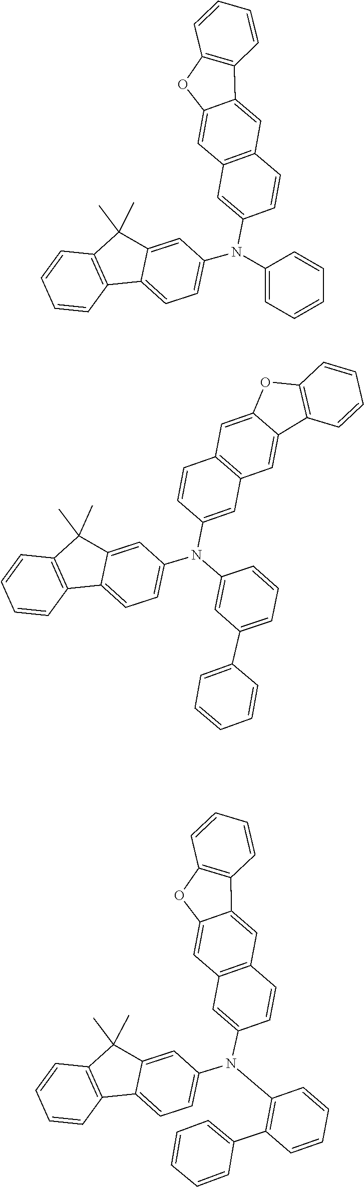 [SMILES: CC1(C)c2ccccc2-c2ccc(N(c3ccccc3)c3ccc4cc5c(cc4c3)oc3ccccc35)cc21 ; CC1(C)c2ccccc2-c2ccc(N(c3cccc(-c4ccccc4)c3)c3ccc4cc5oc6ccccc6c5cc4c3)cc21 ; CC1(C)c2ccccc2-c2ccc(N(c3ccc4cc5c(cc4c3)oc3ccccc35)c3ccccc3-c3ccccc3)cc21]

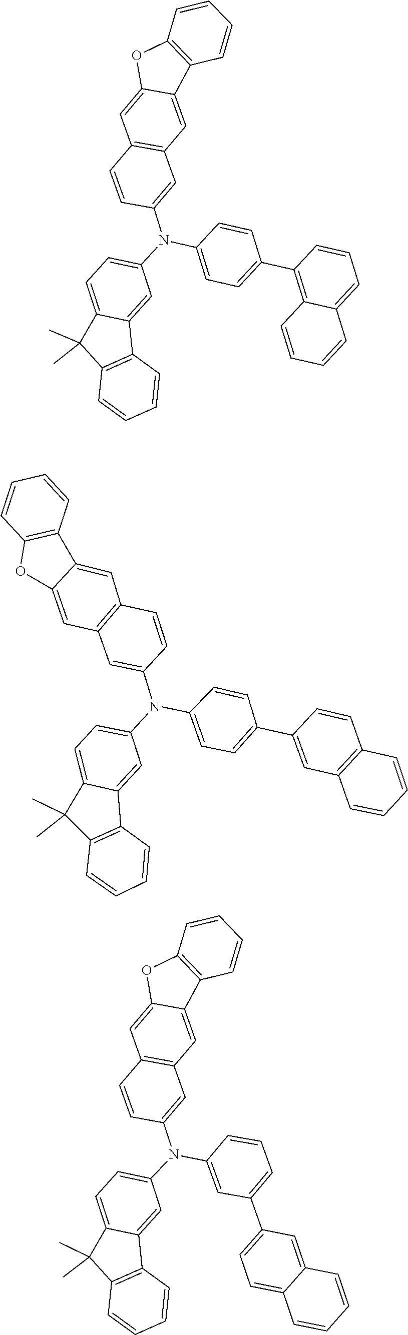 [SMILES: CC1(C)c2ccccc2-c2cc(N(c3ccc(-c4cccc5ccccc45)cc3)c3ccc4cc5oc6ccccc6c5cc4c3)ccc21 ; CC1(C)c2ccccc2-c2cc(N(c3ccc(-c4ccc5ccccc5c4)cc3)c3ccc4cc5c(cc4c3)oc3ccccc35)ccc21 ; CC1(C)c2ccccc2-c2cc(N(c3cccc(-c4ccc5ccccc5c4)c3)c3ccc4cc5oc6ccccc6c5cc4c3)ccc21]

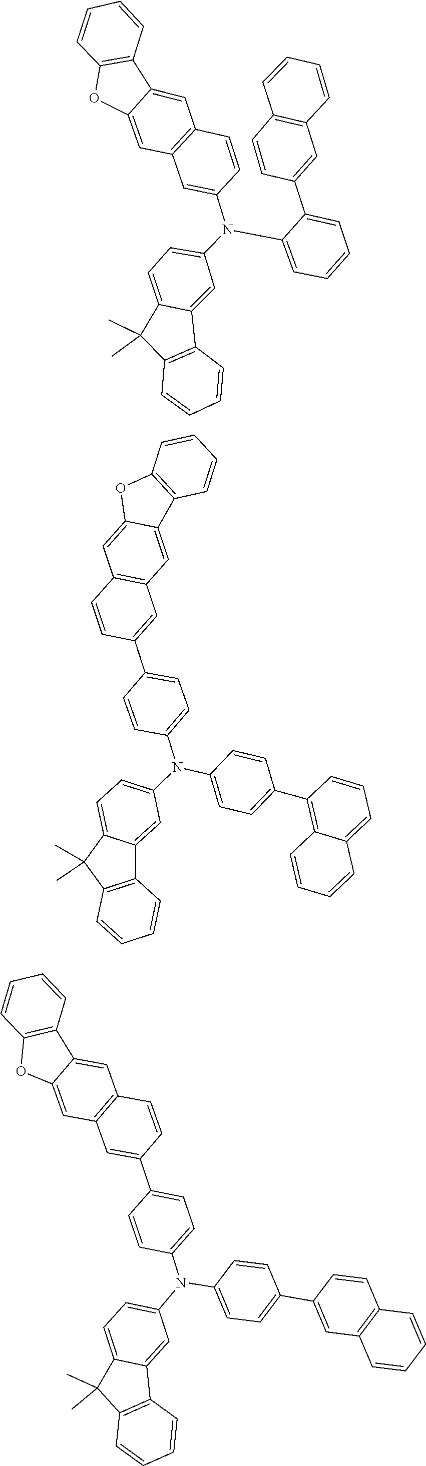

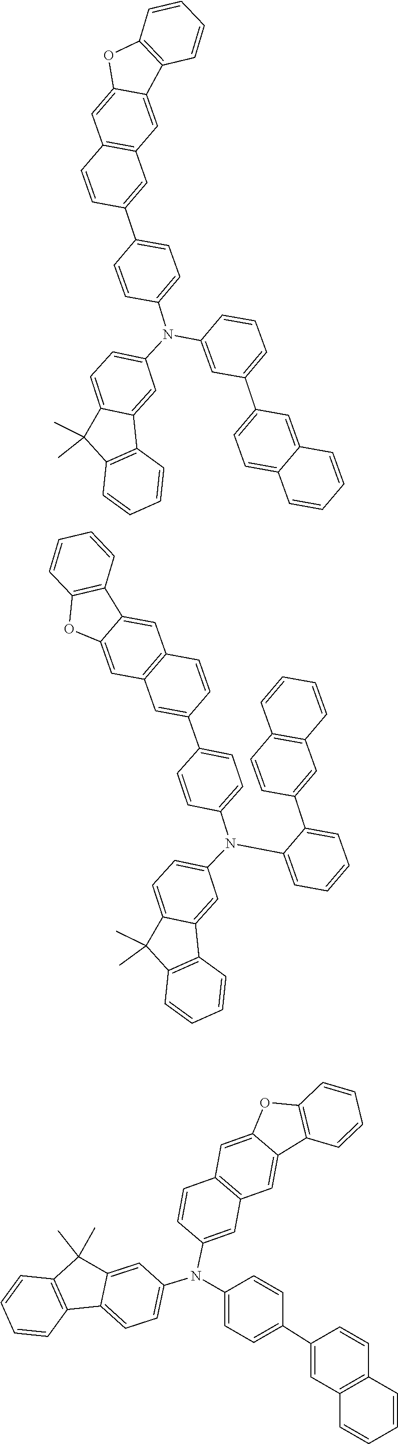

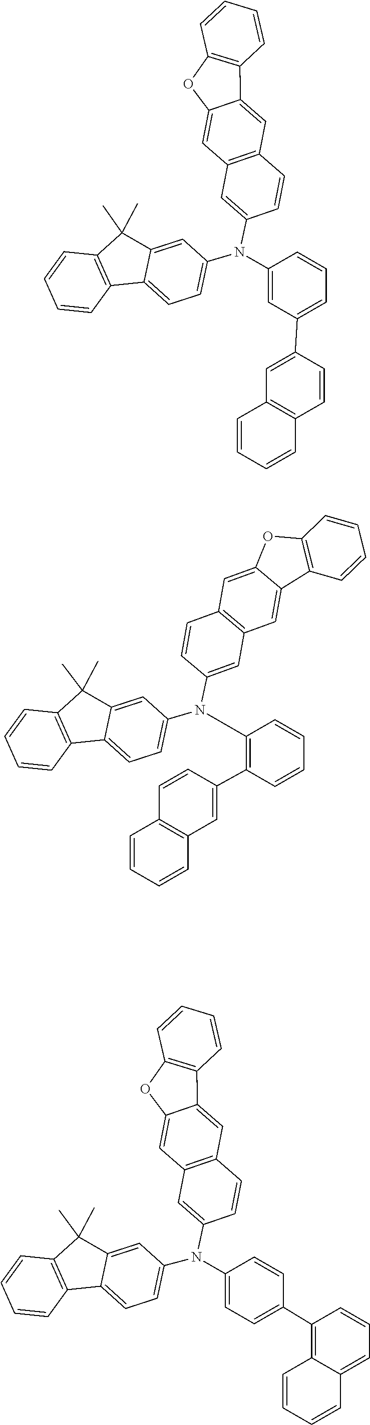 [SMILES: CC1(C)c2ccccc2-c2ccc(N(c3cccc(-c4ccc5ccccc5c4)c3)c3ccc4cc5c(cc4c3)oc3ccccc35)cc21 ; CC1(C)c2ccccc2-c2ccc(N(c3ccc4cc5oc6ccccc6c5cc4c3)c3ccccc3-c3ccc4ccccc4c3)cc21 ; CC1(C)c2ccccc2-c2ccc(N(c3ccc(-c4cccc5ccccc45)cc3)c3ccc4cc5c(cc4c3)oc3ccccc35)cc21]

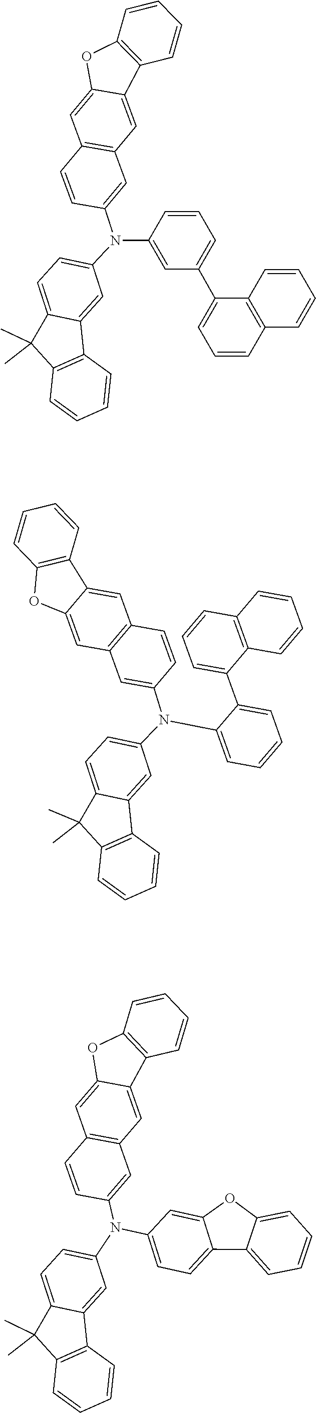 [SMILES: CC1(C)c2ccccc2-c2cc(N(c3cccc(-c4cccc5ccccc45)c3)c3ccc4cc5oc6ccccc6c5cc4c3)ccc21 ; CC1(C)c2ccccc2-c2cc(N(c3ccc4cc5c(cc4c3)oc3ccccc35)c3ccccc3-c3cccc4ccccc34)ccc21 ; CC1(C)c2ccccc2-c2cc(N(c3ccc4cc5oc6ccccc6c5cc4c3)c3ccc4c(c3)oc3ccccc34)ccc21]

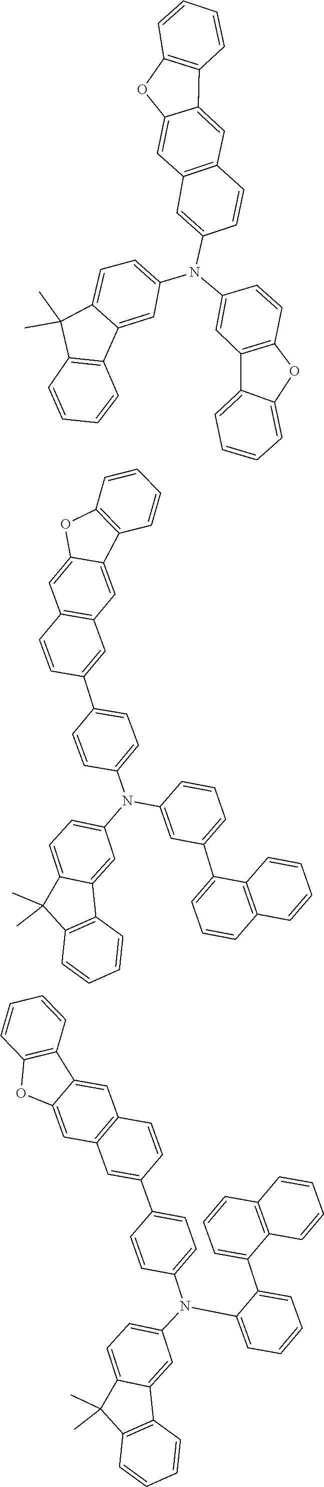

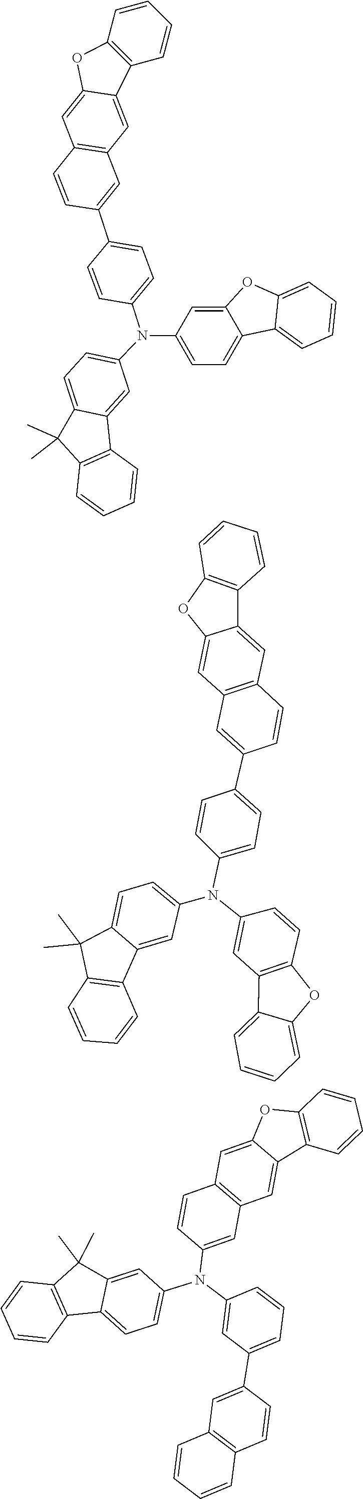

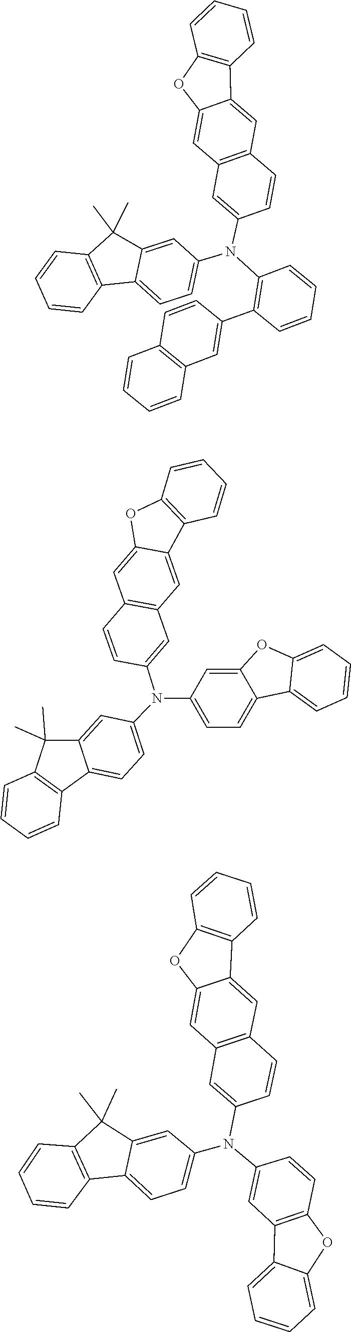 [SMILES: CC1(C)c2ccccc2-c2ccc(N(c3ccc4cc5c(cc4c3)oc3ccccc35)c3ccccc3-c3ccc4ccccc4c3)cc21 ; CC1(C)c2ccccc2-c2ccc(N(c3ccc4cc5oc6ccccc6c5cc4c3)c3ccc4c(c3)oc3ccccc34)cc21 ; CC1(C)c2ccccc2-c2ccc(N(c3ccc4cc5c(cc4c3)oc3ccccc35)c3ccc4oc5ccccc5c4c3)cc21]

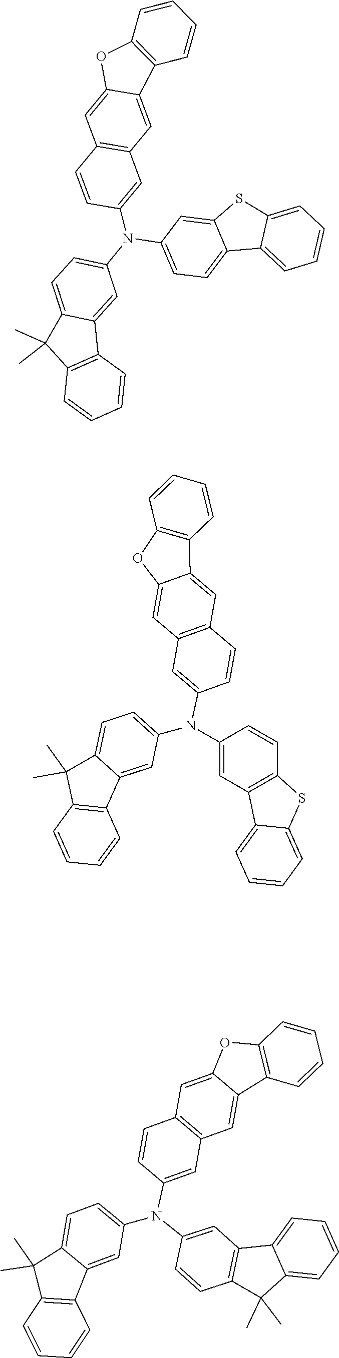 [SMILES: CC1(C)c2ccccc2-c2cc(N(c3ccc4cc5oc6ccccc6c5cc4c3)c3ccc4c(c3)sc3ccccc34)ccc21 ; CC1(C)c2ccccc2-c2cc(N(c3ccc4cc5c(cc4c3)oc3ccccc35)c3ccc4sc5ccccc5c4c3)ccc21 ; CC1(C)c2ccccc2-c2cc(N(c3ccc4c(c3)-c3ccccc3C4(C)C)c3ccc4cc5oc6ccccc6c5cc4c3)ccc21]

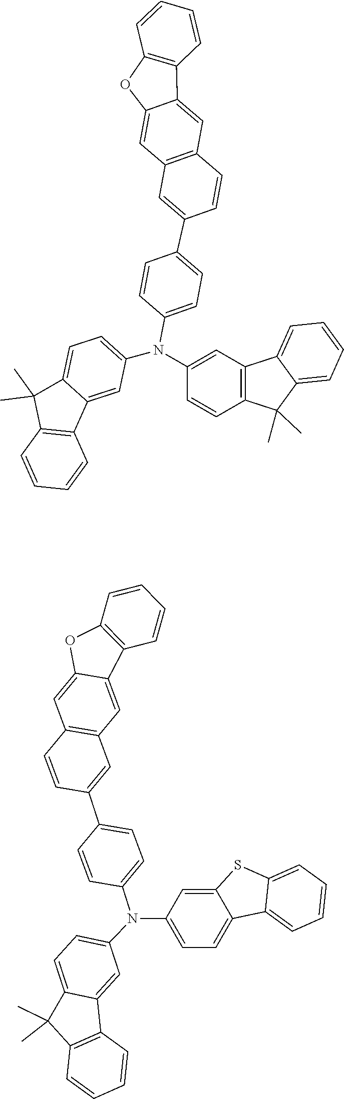 [SMILES: CC1(C)c2ccccc2-c2cc(N(c3ccc(-c4ccc5cc6c(cc5c4)oc4ccccc46)cc3)c3ccc4c(c3)-c3ccccc3C4(C)C)ccc21 ; CC1(C)c2ccccc2-c2cc(N(c3ccc(-c4ccc5cc6oc7ccccc7c6cc5c4)cc3)c3ccc4c(c3)sc3ccccc34)ccc21]

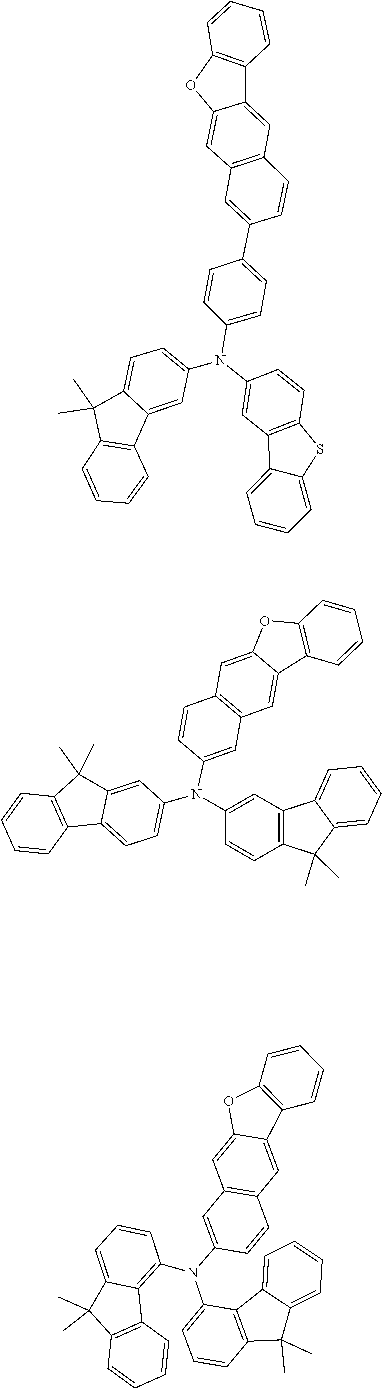 [SMILES: CC1(C)c2ccccc2-c2cc(N(c3ccc(-c4ccc5cc6c(cc5c4)oc4ccccc46)cc3)c3ccc4sc5ccccc5c4c3)ccc21 ; CC1(C)c2ccccc2-c2cc(N(c3ccc4c(c3)C(C)(C)c3ccccc3-4)c3ccc4cc5oc6ccccc6c5cc4c3)ccc21 ; CC1(C)c2ccccc2-c2c(N(c3ccc4cc5c(cc4c3)oc3ccccc35)c3cccc4c3-c3ccccc3C4(C)C)cccc21]

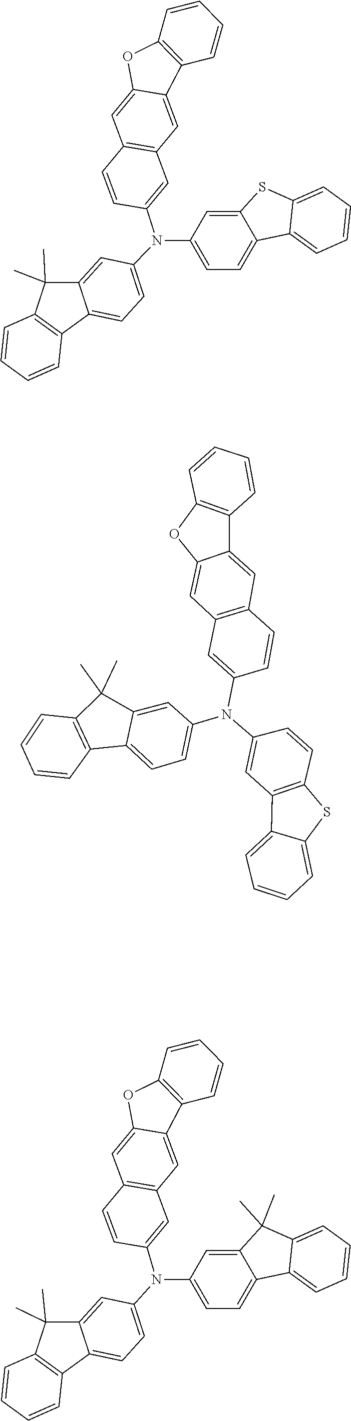 [SMILES: CC1(C)c2ccccc2-c2ccc(N(c3ccc4cc5oc6ccccc6c5cc4c3)c3ccc4c(c3)sc3ccccc34)cc21 ; CC1(C)c2ccccc2-c2ccc(N(c3ccc4cc5c(cc4c3)oc3ccccc35)c3ccc4sc5ccccc5c4c3)cc21 ; CC1(C)c2ccccc2-c2ccc(N(c3ccc4c(c3)C(C)(C)c3ccccc3-4)c3ccc4cc5oc6ccccc6c5cc4c3)cc21]

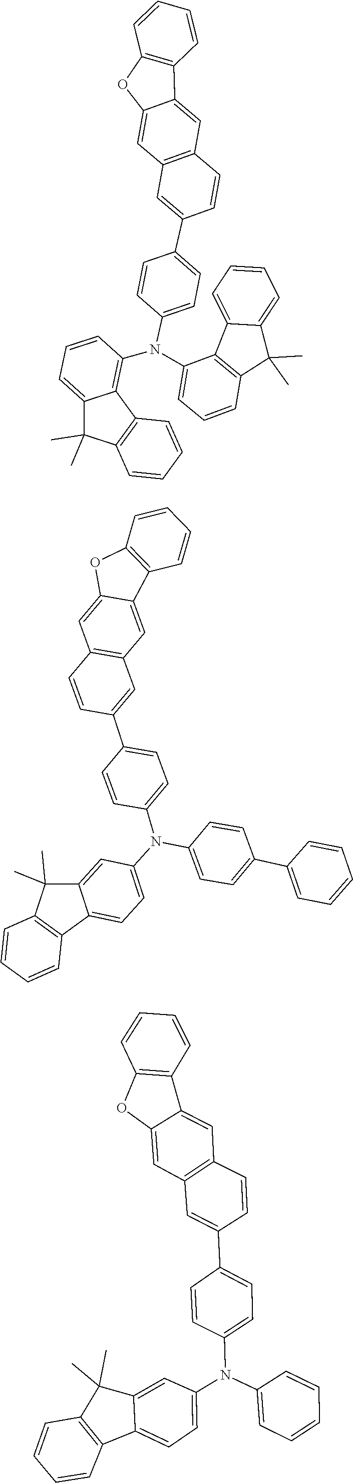

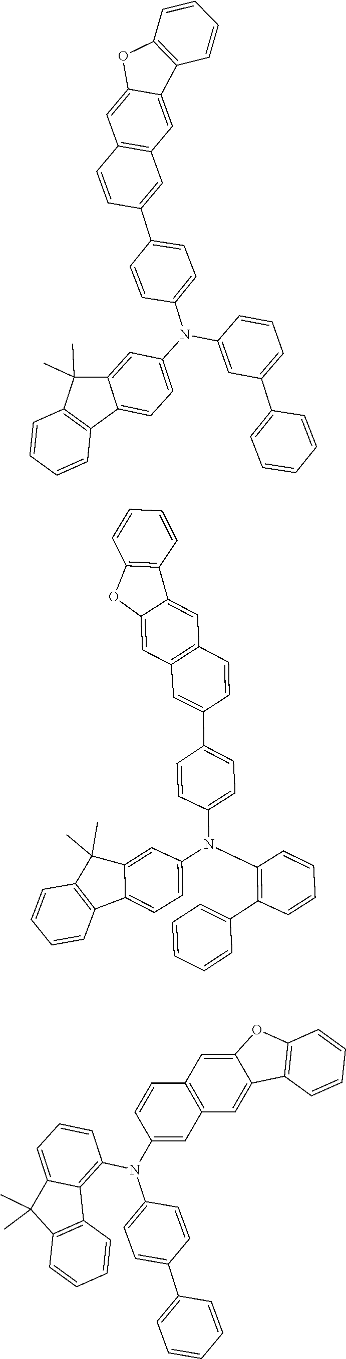 [SMILES: CC1(C)c2ccccc2-c2ccc(N(c3ccc(-c4ccc5cc6oc7ccccc7c6cc5c4)cc3)c3cccc(-c4ccccc4)c3)cc21 ; CC1(C)c2ccccc2-c2ccc(N(c3ccc(-c4ccc5cc6c(cc5c4)oc4ccccc46)cc3)c3ccccc3-c3ccccc3)cc21 ; CC1(C)c2ccccc2-c2c(N(c3ccc(-c4ccccc4)cc3)c3ccc4cc5oc6ccccc6c5cc4c3)cccc21]

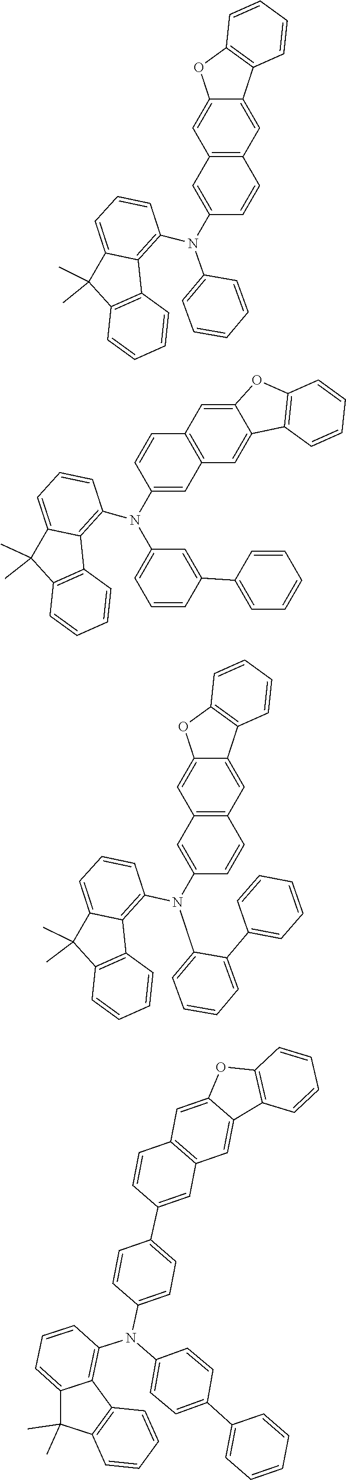 [SMILES: CC1(C)c2ccccc2-c2c(N(c3ccccc3)c3ccc4cc5c(cc4c3)oc3ccccc35)cccc21 ; CC1(C)c2ccccc2-c2c(N(c3cccc(-c4ccccc4)c3)c3ccc4cc5oc6ccccc6c5cc4c3)cccc21 ; CC1(C)c2ccccc2-c2c(N(c3ccc4cc5c(cc4c3)oc3ccccc35)c3ccccc3-c3ccccc3)cccc21 ; CC1(C)c2ccccc2-c2c(N(c3ccc(-c4ccccc4)cc3)c3ccc(-c4ccc5cc6oc7ccccc7c6cc5c4)cc3)cccc21]

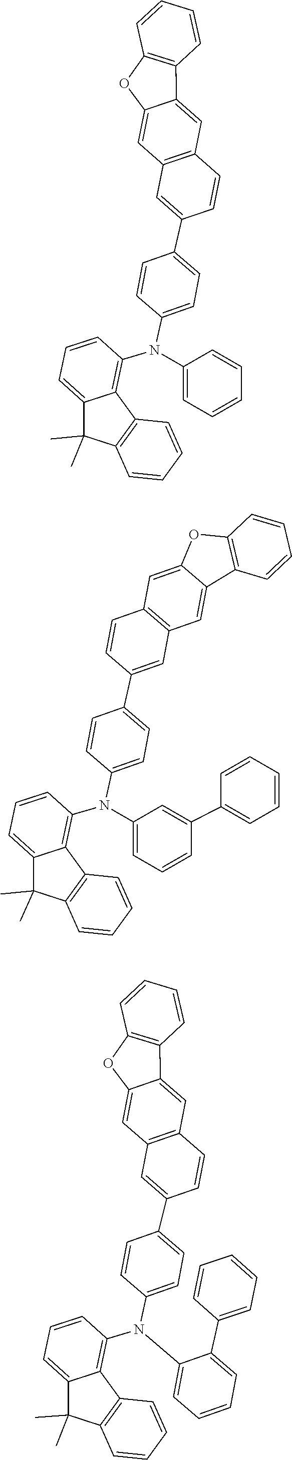 [SMILES: CC1(C)c2ccccc2-c2c(N(c3ccccc3)c3ccc(-c4ccc5cc6c(cc5c4)oc4ccccc46)cc3)cccc21 ; CC1(C)c2ccccc2-c2c(N(c3ccc(-c4ccc5cc6oc7ccccc7c6cc5c4)cc3)c3cccc(-c4ccccc4)c3)cccc21 ; CC1(C)c2ccccc2-c2c(N(c3ccc(-c4ccc5cc6c(cc5c4)oc4ccccc46)cc3)c3ccccc3-c3ccccc3)cccc21]

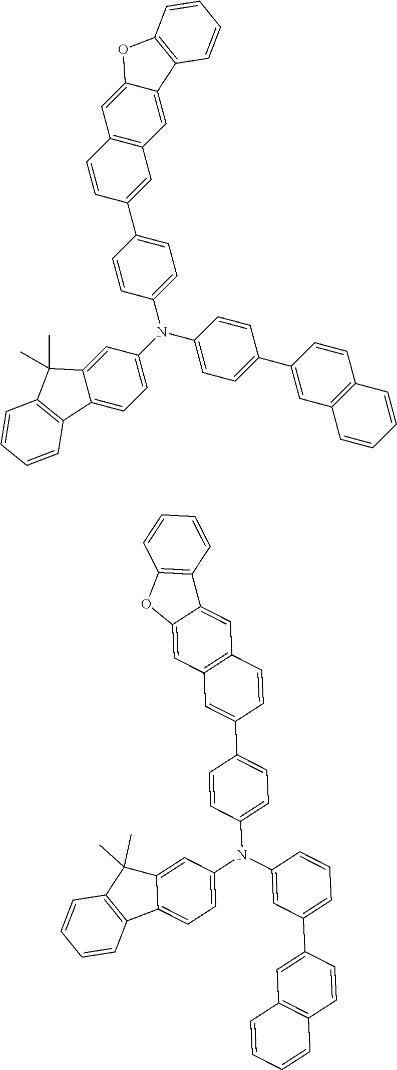 [SMILES: CC1(C)c2ccccc2-c2ccc(N(c3ccc(-c4ccc5ccccc5c4)cc3)c3ccc(-c4ccc5cc6oc7ccccc7c6cc5c4)cc3)cc21 ; CC1(C)c2ccccc2-c2ccc(N(c3ccc(-c4ccc5cc6c(cc5c4)oc4ccccc46)cc3)c3cccc(-c4ccc5ccccc5c4)c3)cc21]

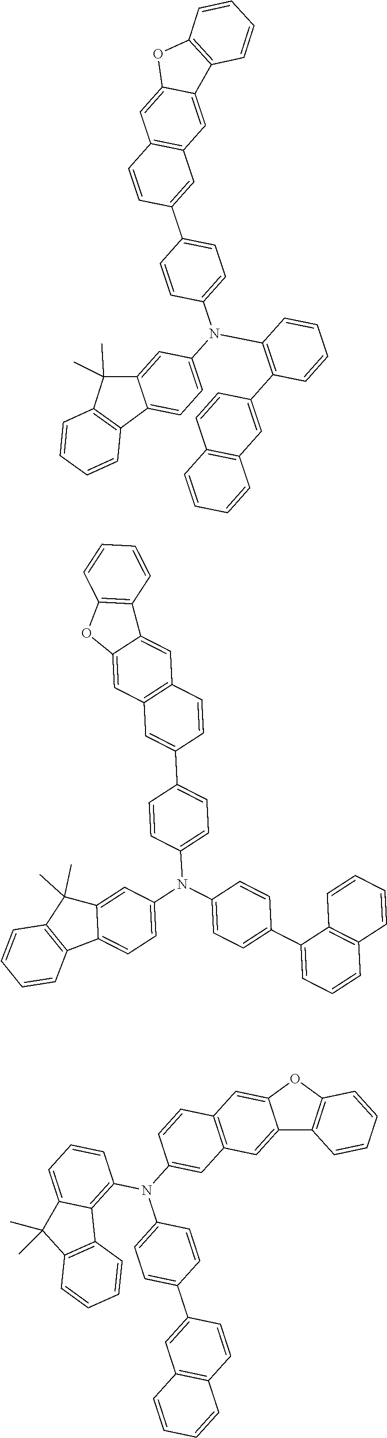 [SMILES: CC1(C)c2ccccc2-c2ccc(N(c3ccc(-c4ccc5cc6oc7ccccc7c6cc5c4)cc3)c3ccccc3-c3ccc4ccccc4c3)cc21 ; CC1(C)c2ccccc2-c2ccc(N(c3ccc(-c4ccc5cc6c(cc5c4)oc4ccccc46)cc3)c3ccc(-c4cccc5ccccc45)cc3)cc21 ; CC1(C)c2ccccc2-c2c(N(c3ccc(-c4ccc5ccccc5c4)cc3)c3ccc4cc5oc6ccccc6c5cc4c3)cccc21]

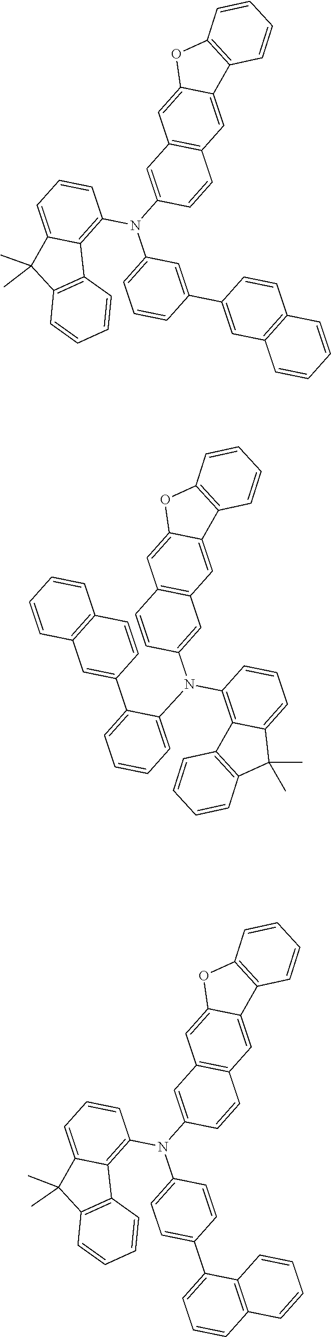

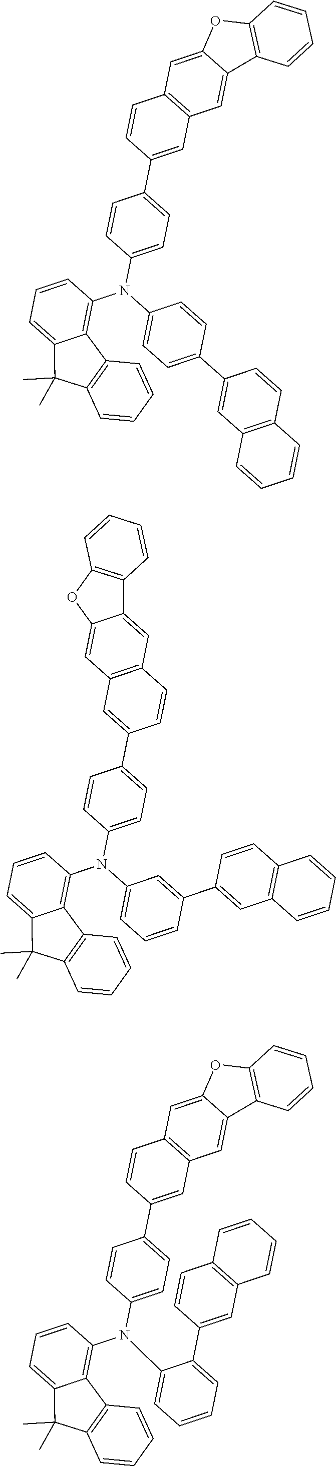 [SMILES: CC1(C)c2ccccc2-c2c(N(c3ccc(-c4ccc5ccccc5c4)cc3)c3ccc(-c4ccc5cc6oc7ccccc7c6cc5c4)cc3)cccc21 ; CC1(C)c2ccccc2-c2c(N(c3ccc(-c4ccc5cc6c(cc5c4)oc4ccccc46)cc3)c3cccc(-c4ccc5ccccc5c4)c3)cccc21 ; CC1(C)c2ccccc2-c2c(N(c3ccc(-c4ccc5cc6oc7ccccc7c6cc5c4)cc3)c3ccccc3-c3ccc4ccccc4c3)cccc21]

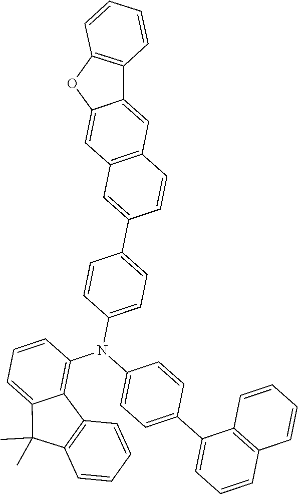 [SMILES: CC1(C)c2ccccc2-c2c(N(c3ccc(-c4ccc5cc6c(cc5c4)oc4ccccc46)cc3)c3ccc(-c4cccc5ccccc45)cc3)cccc21]

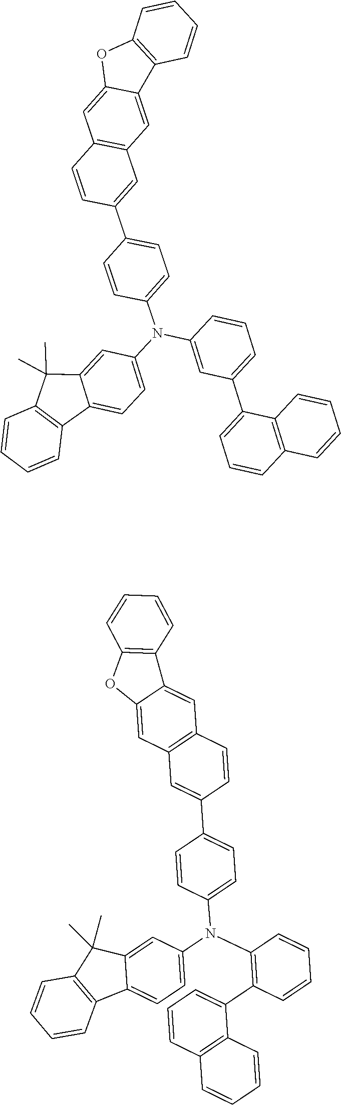 [SMILES: CC1(C)c2ccccc2-c2ccc(N(c3ccc(-c4ccc5cc6oc7ccccc7c6cc5c4)cc3)c3cccc(-c4cccc5ccccc45)c3)cc21 ; CC1(C)c2ccccc2-c2ccc(N(c3ccc(-c4ccc5cc6c(cc5c4)oc4ccccc46)cc3)c3ccccc3-c3cccc4ccccc34)cc21]

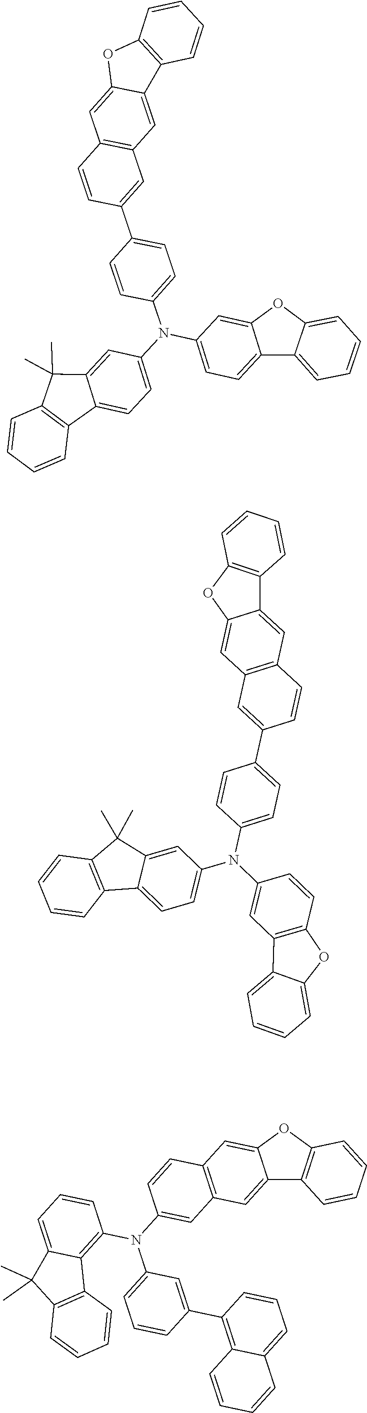 [SMILES: CC1(C)c2ccccc2-c2ccc(N(c3ccc(-c4ccc5cc6oc7ccccc7c6cc5c4)cc3)c3ccc4c(c3)oc3ccccc34)cc21 ; CC1(C)c2ccccc2-c2ccc(N(c3ccc(-c4ccc5cc6c(cc5c4)oc4ccccc46)cc3)c3ccc4oc5ccccc5c4c3)cc21 ; CC1(C)c2ccccc2-c2c(N(c3cccc(-c4cccc5ccccc45)c3)c3ccc4cc5oc6ccccc6c5cc4c3)cccc21]

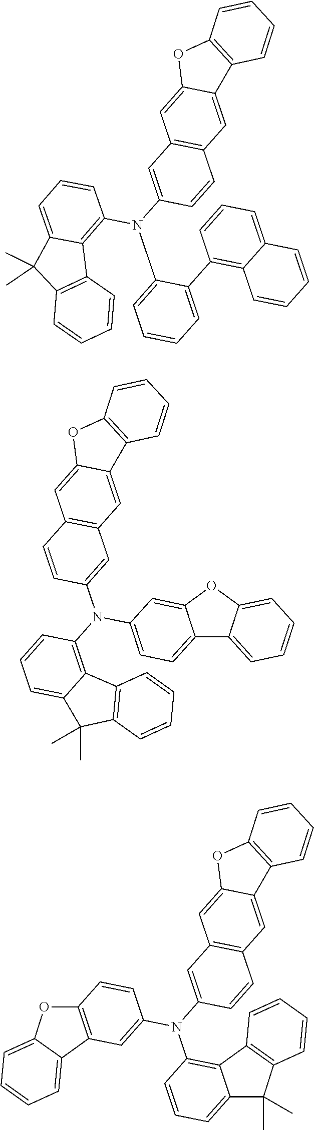

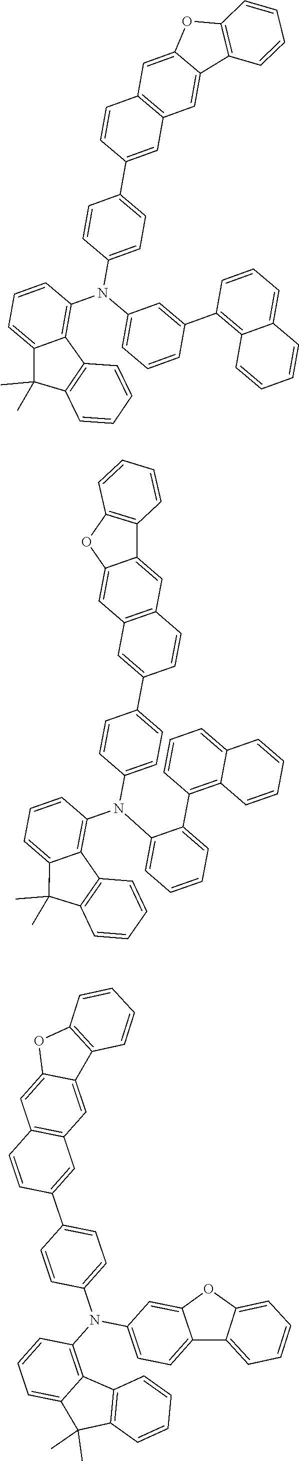

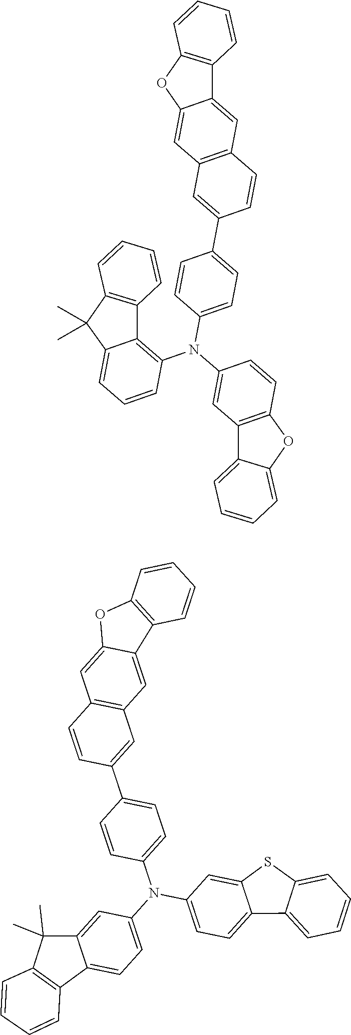 [SMILES: CC1(C)c2ccccc2-c2c(N(c3ccc(-c4ccc5cc6c(cc5c4)oc4ccccc46)cc3)c3ccc4oc5ccccc5c4c3)cccc21 ; CC1(C)c2ccccc2-c2ccc(N(c3ccc(-c4ccc5cc6oc7ccccc7c6cc5c4)cc3)c3ccc4c(c3)sc3ccccc34)cc21]

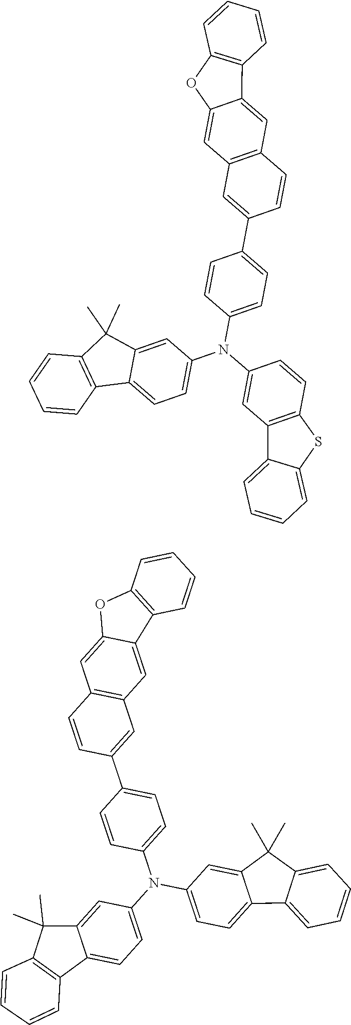 [SMILES: CC1(C)c2ccccc2-c2ccc(N(c3ccc(-c4ccc5cc6c(cc5c4)oc4ccccc46)cc3)c3ccc4sc5ccccc5c4c3)cc21 ; CC1(C)c2ccccc2-c2ccc(N(c3ccc(-c4ccc5cc6oc7ccccc7c6cc5c4)cc3)c3ccc4c(c3)C(C)(C)c3ccccc3-4)cc21]

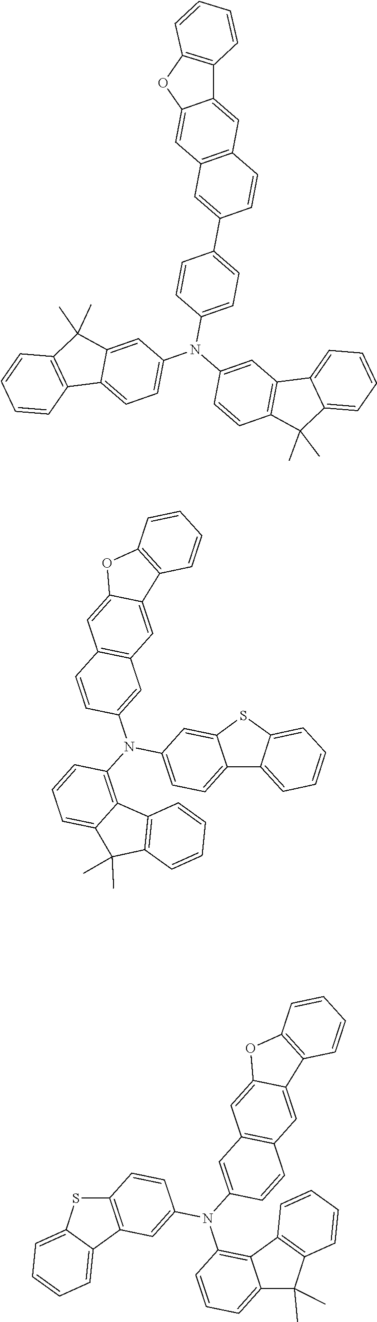 [SMILES: CC1(C)c2ccccc2-c2cc(N(c3ccc(-c4ccc5cc6c(cc5c4)oc4ccccc46)cc3)c3ccc4c(c3)C(C)(C)c3ccccc3-4)ccc21 ; CC1(C)c2ccccc2-c2c(N(c3ccc4cc5oc6ccccc6c5cc4c3)c3ccc4c(c3)sc3ccccc34)cccc21 ; CC1(C)c2ccccc2-c2c(N(c3ccc4cc5c(cc4c3)oc3ccccc35)c3ccc4sc5ccccc5c4c3)cccc21]

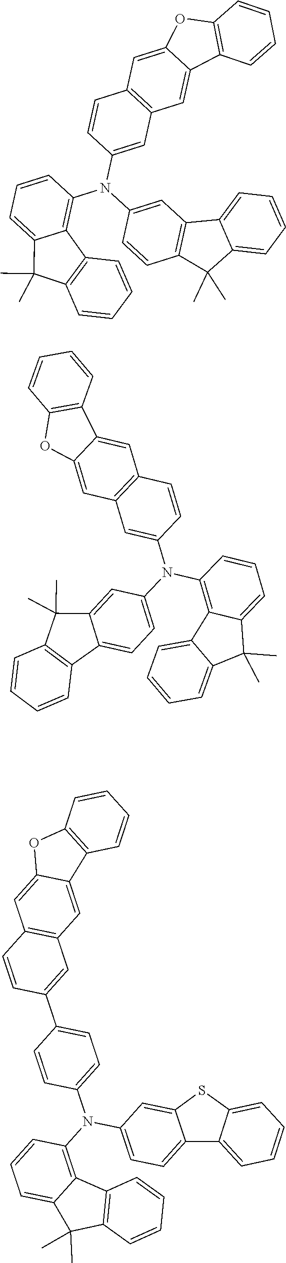

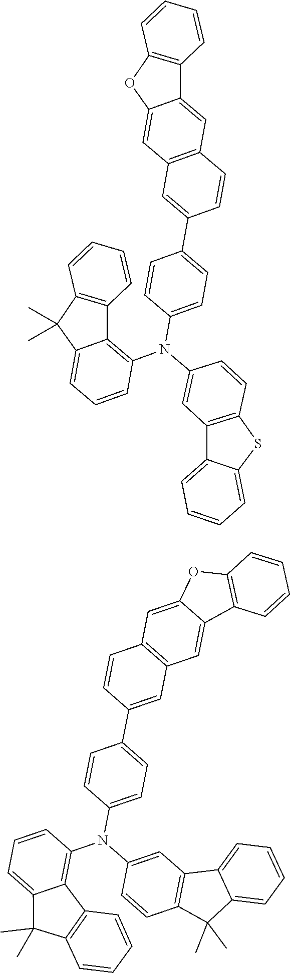

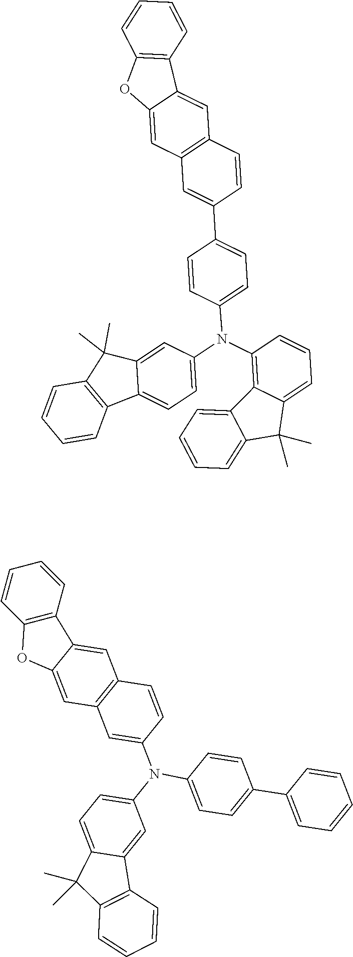 [SMILES: CC1(C)c2ccccc2-c2ccc(N(c3ccc(-c4ccc5cc6c(cc5c4)oc4ccccc46)cc3)c3cccc4c3-c3ccccc3C4(C)C)cc21 ; CC1(C)c2ccccc2-c2cc(N(c3ccc(-c4ccccc4)cc3)c3ccc4cc5c(cc4c3)oc3ccccc35)ccc21]

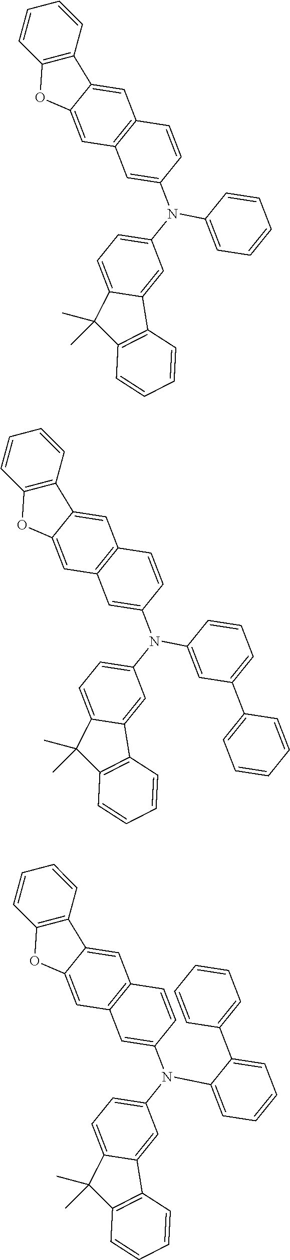 [SMILES: CC1(C)c2ccccc2-c2cc(N(c3ccccc3)c3ccc4cc5c(cc4c3)oc3ccccc35)ccc21 ; CC1(C)c2ccccc2-c2cc(N(c3cccc(-c4ccccc4)c3)c3ccc4cc5c(cc4c3)oc3ccccc35)ccc21 ; CC1(C)c2ccccc2-c2cc(N(c3ccc4cc5c(cc4c3)oc3ccccc35)c3ccccc3-c3ccccc3)ccc21]

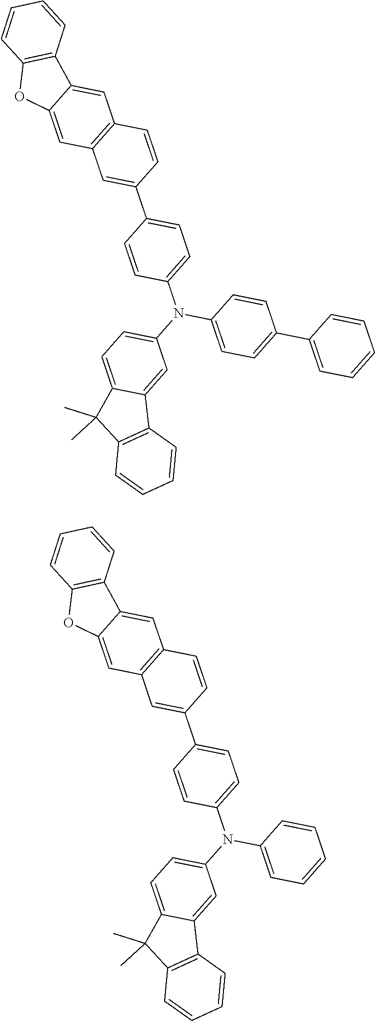 [SMILES: CC1(C)c2ccccc2-c2cc(N(c3ccc(-c4ccccc4)cc3)c3ccc(-c4ccc5cc6c(cc5c4)oc4ccccc46)cc3)ccc21 ; CC1(C)c2ccccc2-c2cc(N(c3ccccc3)c3ccc(-c4ccc5cc6c(cc5c4)oc4ccccc46)cc3)ccc21]

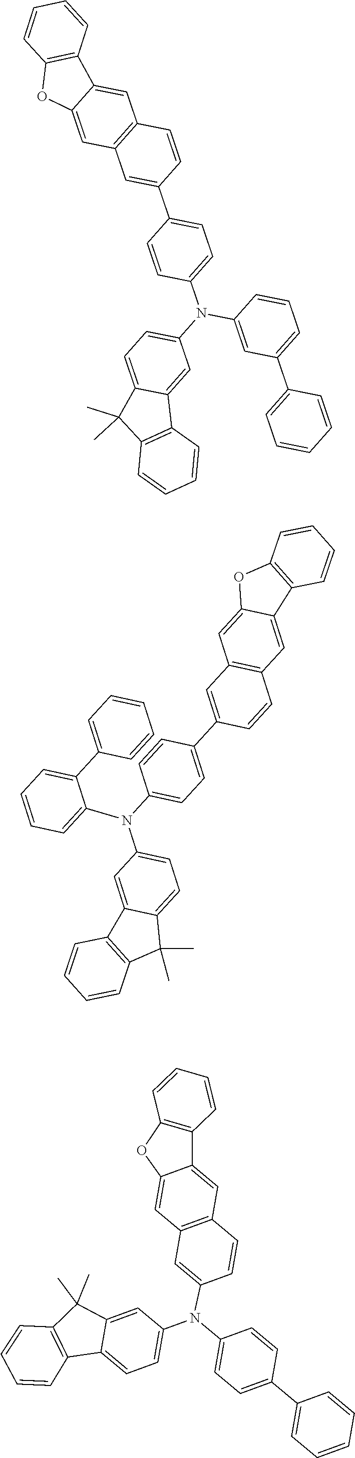 [SMILES: CC1(C)c2ccccc2-c2cc(N(c3ccc(-c4ccc5cc6c(cc5c4)oc4ccccc46)cc3)c3cccc(-c4ccccc4)c3)ccc21 ; CC1(C)c2ccccc2-c2cc(N(c3ccc(-c4ccc5cc6c(cc5c4)oc4ccccc46)cc3)c3ccccc3-c3ccccc3)ccc21 ; CC1(C)c2ccccc2-c2ccc(N(c3ccc(-c4ccccc4)cc3)c3ccc4cc5c(cc4c3)oc3ccccc35)cc21]

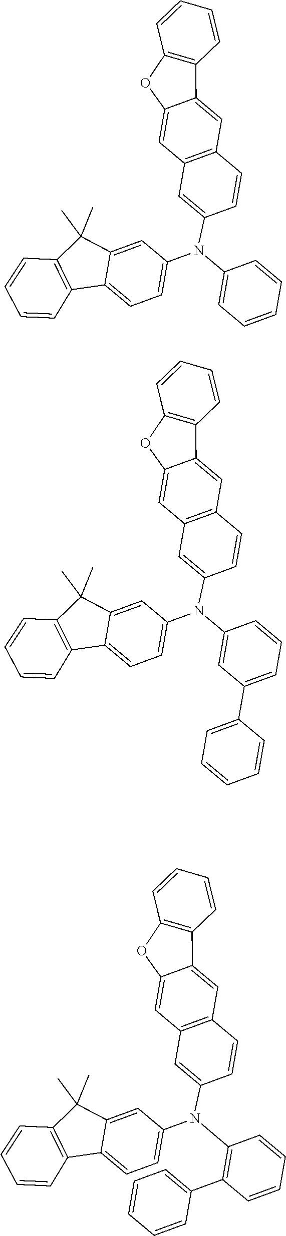 [SMILES: CC1(C)c2ccccc2-c2ccc(N(c3ccccc3)c3ccc4cc5c(cc4c3)oc3ccccc35)cc21 ; CC1(C)c2ccccc2-c2ccc(N(c3cccc(-c4ccccc4)c3)c3ccc4cc5c(cc4c3)oc3ccccc35)cc21 ; CC1(C)c2ccccc2-c2ccc(N(c3ccc4cc5c(cc4c3)oc3ccccc35)c3ccccc3-c3ccccc3)cc21]

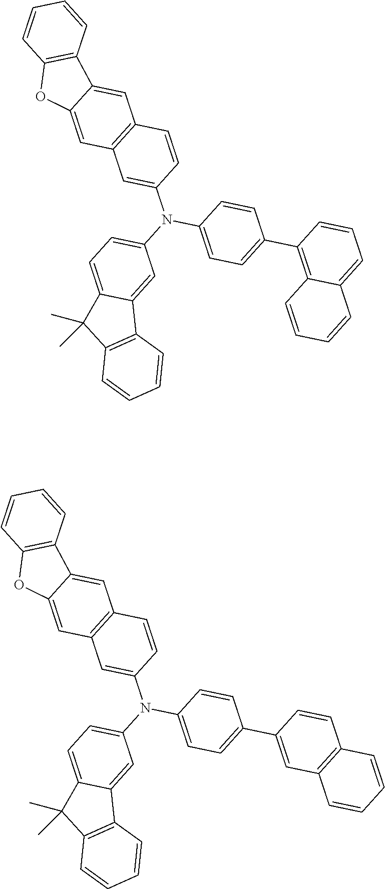 [SMILES: CC1(C)c2ccccc2-c2cc(N(c3ccc(-c4cccc5ccccc45)cc3)c3ccc4cc5c(cc4c3)oc3ccccc35)ccc21 ; CC1(C)c2ccccc2-c2cc(N(c3ccc(-c4ccc5ccccc5c4)cc3)c3ccc4cc5c(cc4c3)oc3ccccc35)ccc21]

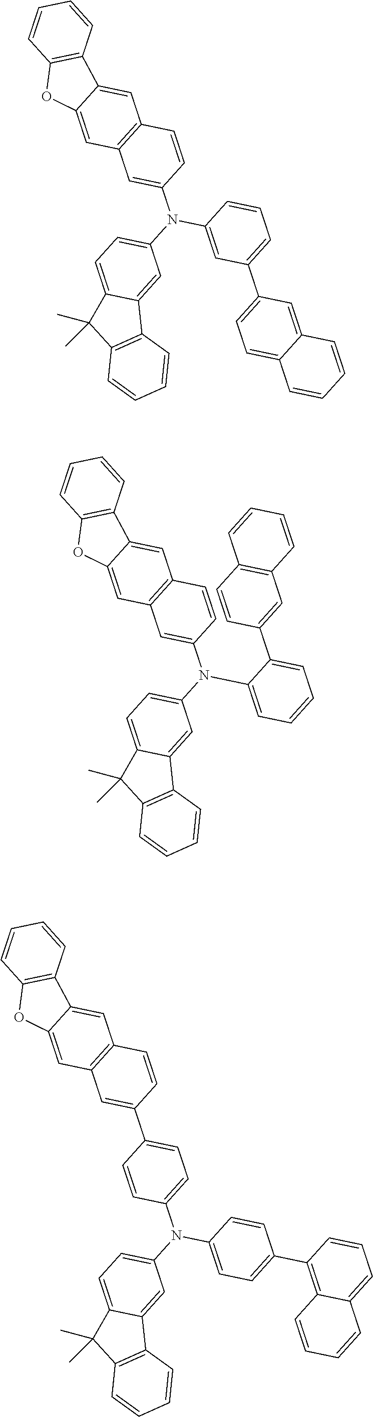 [SMILES: CC1(C)c2ccccc2-c2cc(N(c3cccc(-c4ccc5ccccc5c4)c3)c3ccc4cc5c(cc4c3)oc3ccccc35)ccc21 ; CC1(C)c2ccccc2-c2cc(N(c3ccc4cc5c(cc4c3)oc3ccccc35)c3ccccc3-c3ccc4ccccc4c3)ccc21 ; CC1(C)c2ccccc2-c2cc(N(c3ccc(-c4ccc5cc6c(cc5c4)oc4ccccc46)cc3)c3ccc(-c4cccc5ccccc45)cc3)ccc21]

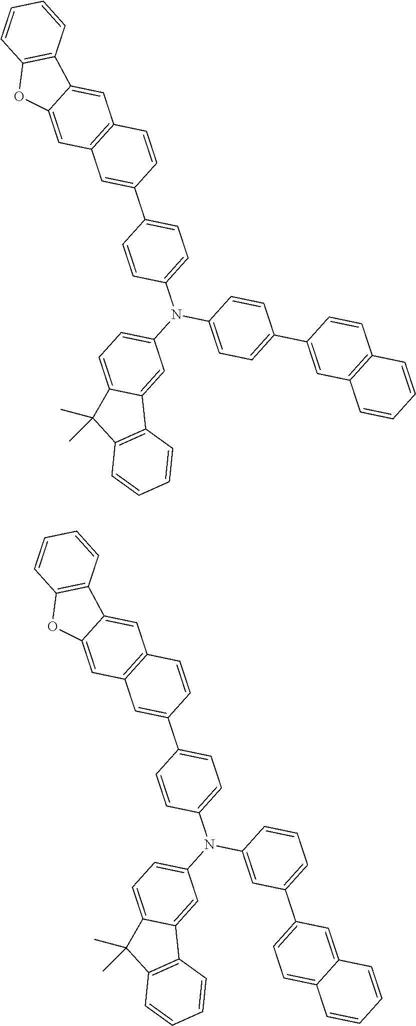 [SMILES: CC1(C)c2ccccc2-c2cc(N(c3ccc(-c4ccc5ccccc5c4)cc3)c3ccc(-c4ccc5cc6c(cc5c4)oc4ccccc46)cc3)ccc21 ; CC1(C)c2ccccc2-c2cc(N(c3ccc(-c4ccc5cc6c(cc5c4)oc4ccccc46)cc3)c3cccc(-c4ccc5ccccc5c4)c3)ccc21]

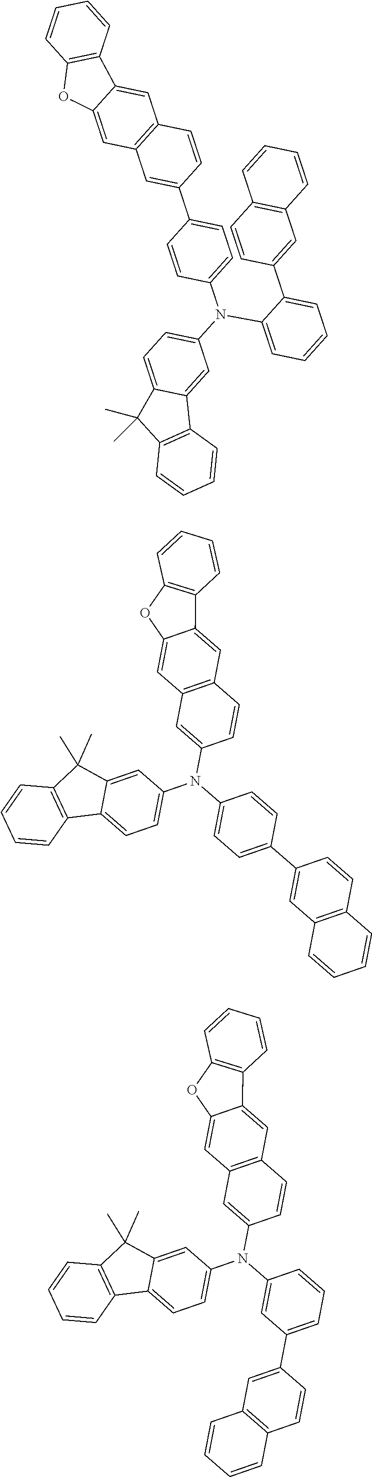

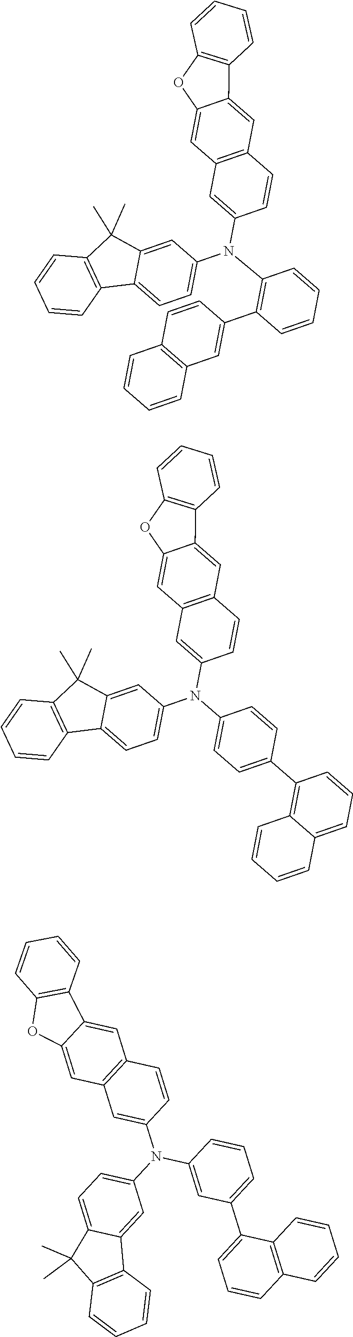

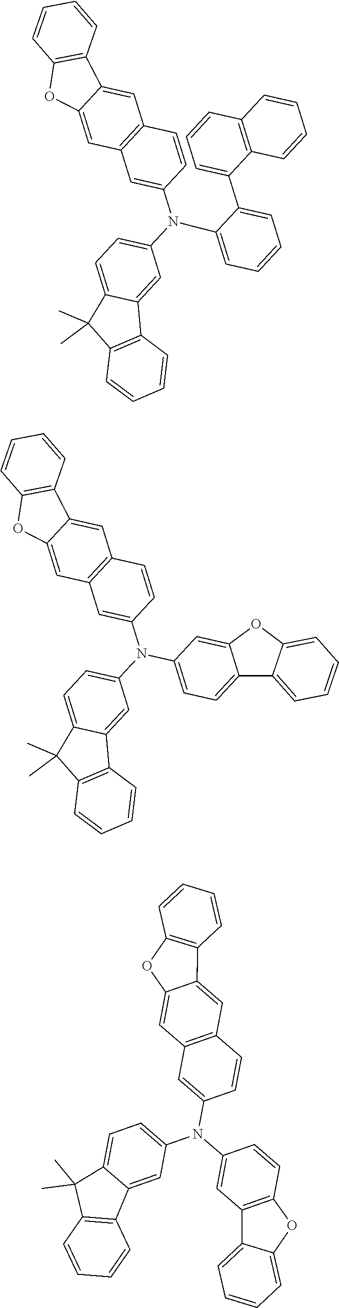 [SMILES: CC1(C)c2ccccc2-c2cc(N(c3ccc4cc5c(cc4c3)oc3ccccc35)c3ccccc3-c3cccc4ccccc34)ccc21 ; CC1(C)c2ccccc2-c2cc(N(c3ccc4cc5c(cc4c3)oc3ccccc35)c3ccc4c(c3)oc3ccccc34)ccc21 ; CC1(C)c2ccccc2-c2cc(N(c3ccc4cc5c(cc4c3)oc3ccccc35)c3ccc4oc5ccccc5c4c3)ccc21]

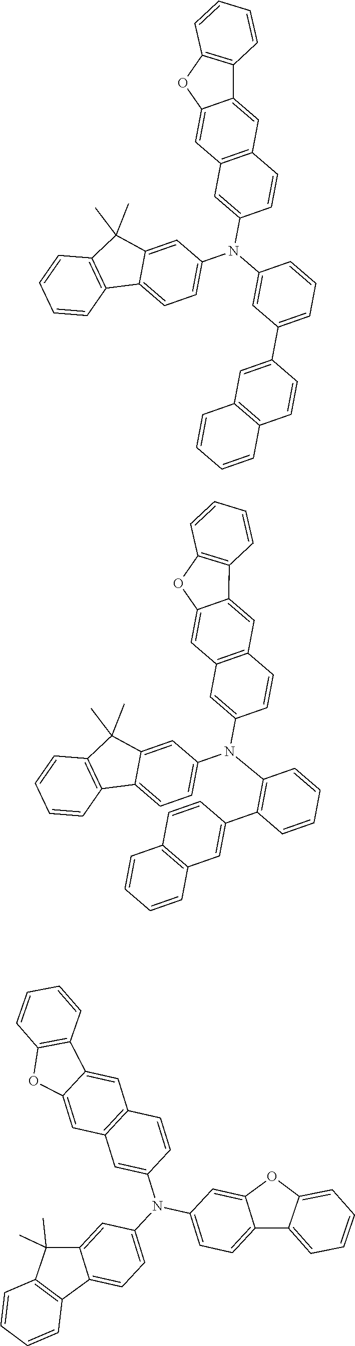

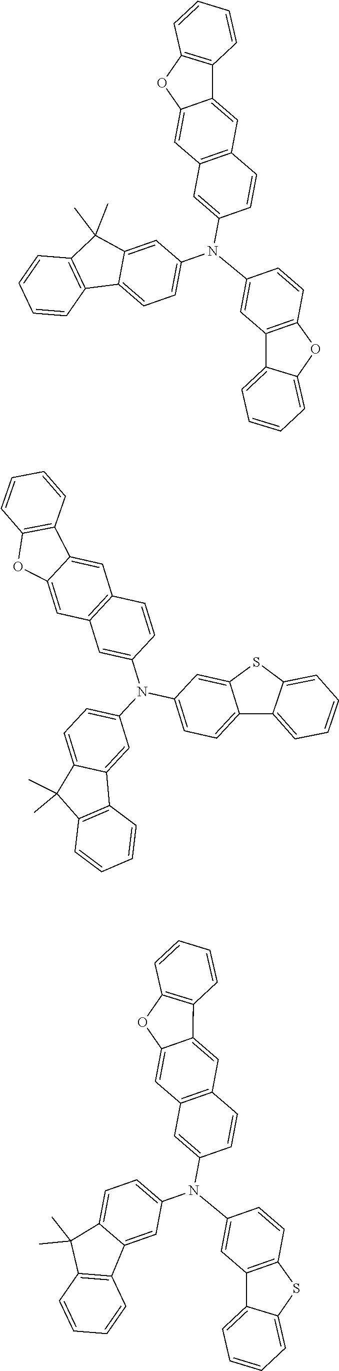 [SMILES: CC1(C)c2ccccc2-c2ccc(N(c3ccc4cc5c(cc4c3)oc3ccccc35)c3ccc4oc5ccccc5c4c3)cc21 ; CC1(C)c2ccccc2-c2cc(N(c3ccc4cc5c(cc4c3)oc3ccccc35)c3ccc4c(c3)sc3ccccc34)ccc21 ; CC1(C)c2ccccc2-c2cc(N(c3ccc4cc5c(cc4c3)oc3ccccc35)c3ccc4sc5ccccc5c4c3)ccc21]

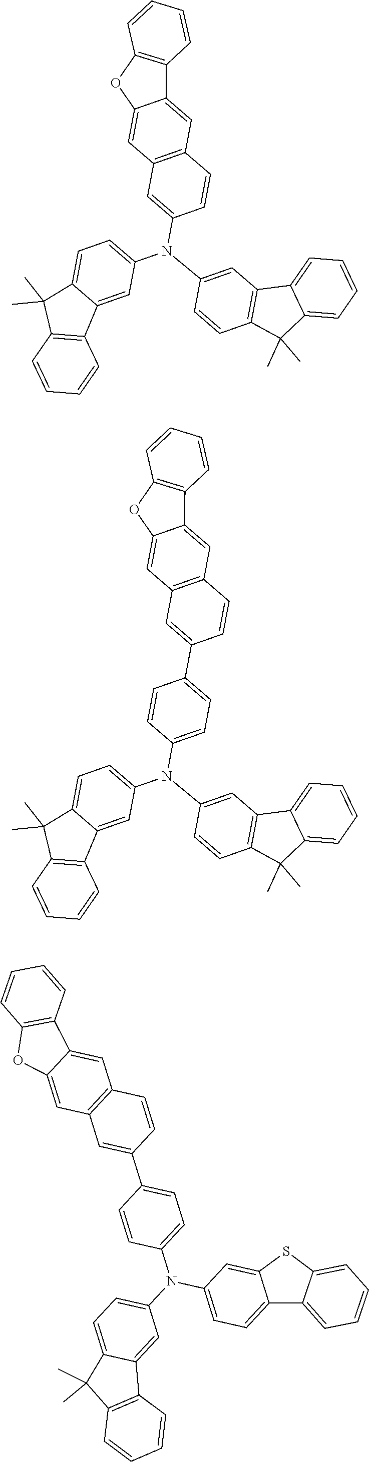 [SMILES: CC1(C)c2ccccc2-c2cc(N(c3ccc4c(c3)-c3ccccc3C4(C)C)c3ccc4cc5c(cc4c3)oc3ccccc35)ccc21 ; CC1(C)c2ccccc2-c2cc(N(c3ccc(-c4ccc5cc6c(cc5c4)oc4ccccc46)cc3)c3ccc4c(c3)-c3ccccc3C4(C)C)ccc21 ; CC1(C)c2ccccc2-c2cc(N(c3ccc(-c4ccc5cc6c(cc5c4)oc4ccccc46)cc3)c3ccc4c(c3)sc3ccccc34)ccc21]

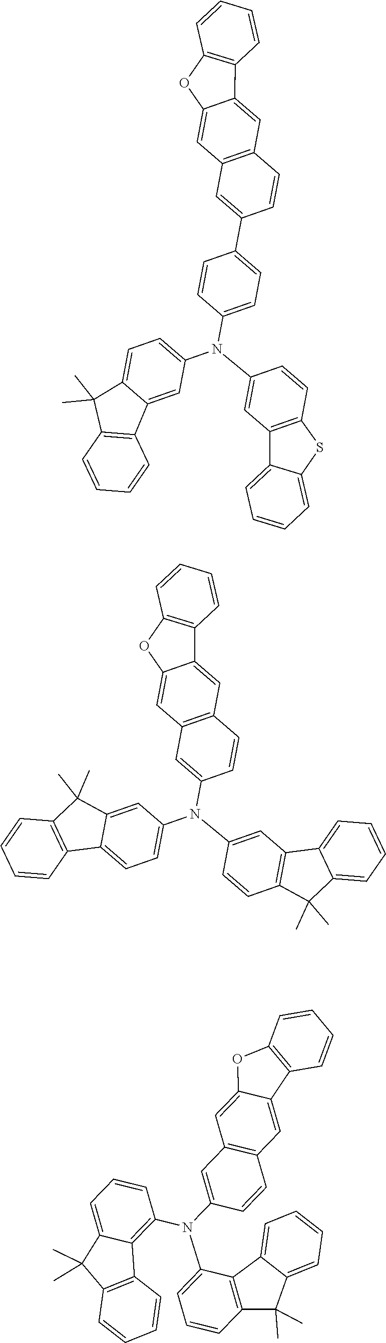 [SMILES: CC1(C)c2ccccc2-c2cc(N(c3ccc(-c4ccc5cc6c(cc5c4)oc4ccccc46)cc3)c3ccc4sc5ccccc5c4c3)ccc21 ; CC1(C)c2ccccc2-c2cc(N(c3ccc4c(c3)C(C)(C)c3ccccc3-4)c3ccc4cc5c(cc4c3)oc3ccccc35)ccc21 ; CC1(C)c2ccccc2-c2c(N(c3ccc4cc5c(cc4c3)oc3ccccc35)c3cccc4c3-c3ccccc3C4(C)C)cccc21]

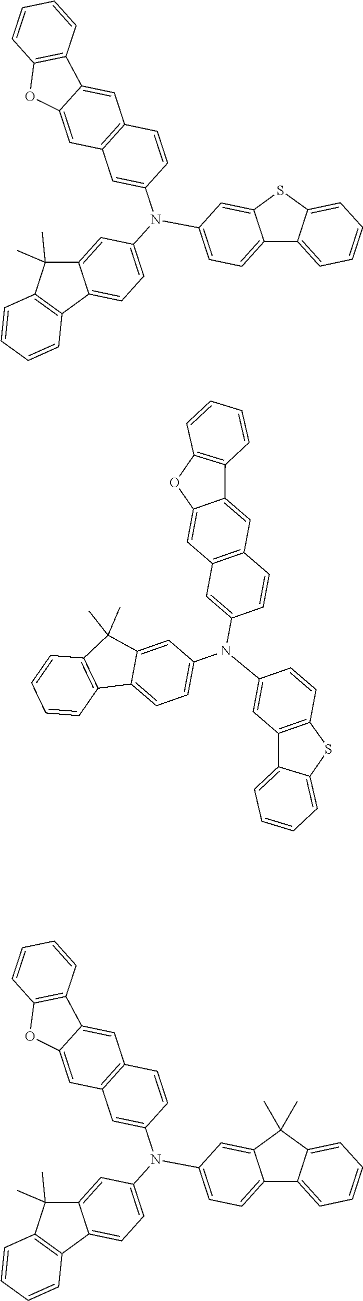 [SMILES: CC1(C)c2ccccc2-c2ccc(N(c3ccc4cc5c(cc4c3)oc3ccccc35)c3ccc4c(c3)sc3ccccc34)cc21 ; CC1(C)c2ccccc2-c2ccc(N(c3ccc4cc5c(cc4c3)oc3ccccc35)c3ccc4sc5ccccc5c4c3)cc21 ; CC1(C)c2ccccc2-c2ccc(N(c3ccc4c(c3)C(C)(C)c3ccccc3-4)c3ccc4cc5c(cc4c3)oc3ccccc35)cc21]

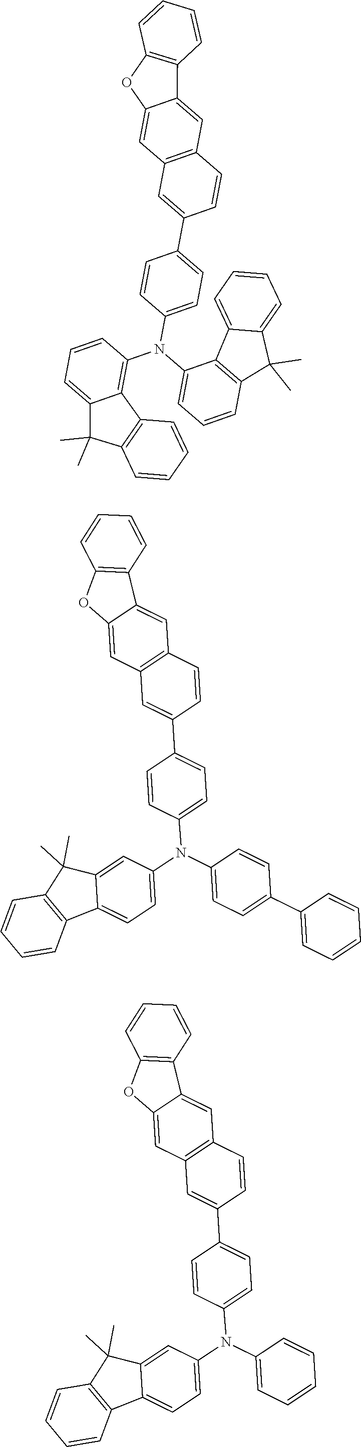

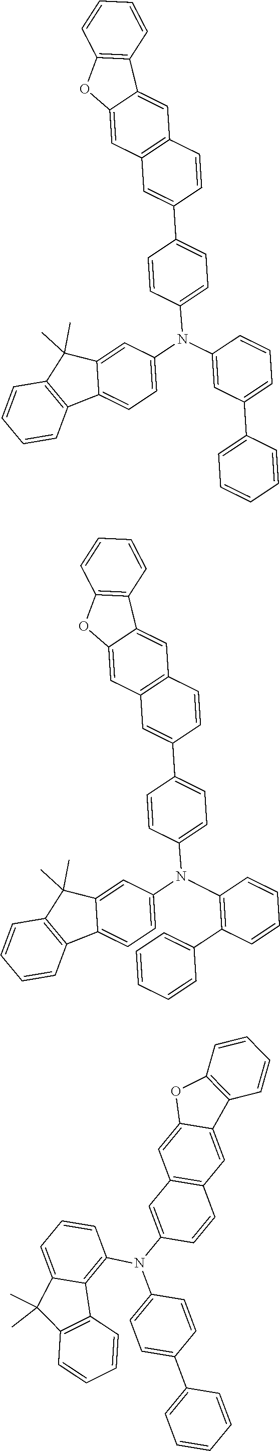 [SMILES: CC1(C)c2ccccc2-c2ccc(N(c3ccc(-c4ccc5cc6c(cc5c4)oc4ccccc46)cc3)c3cccc(-c4ccccc4)c3)cc21 ; CC1(C)c2ccccc2-c2ccc(N(c3ccc(-c4ccc5cc6c(cc5c4)oc4ccccc46)cc3)c3ccccc3-c3ccccc3)cc21 ; CC1(C)c2ccccc2-c2c(N(c3ccc(-c4ccccc4)cc3)c3ccc4cc5c(cc4c3)oc3ccccc35)cccc21]

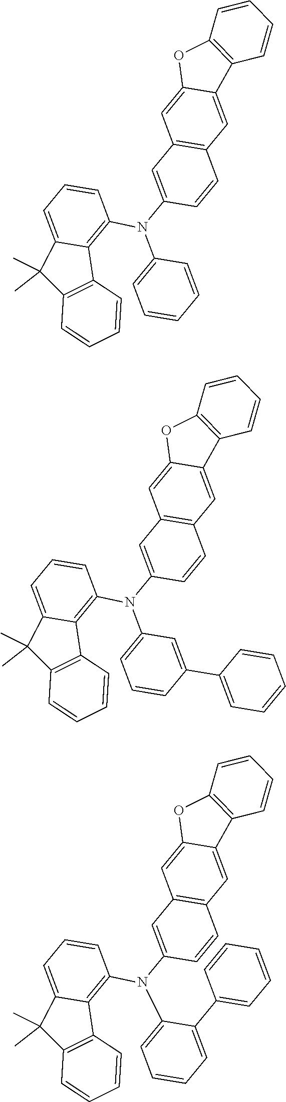

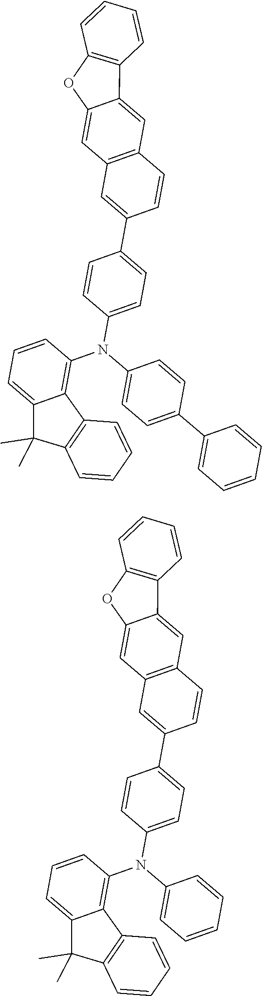 [SMILES: CC1(C)c2ccccc2-c2c(N(c3ccc(-c4ccccc4)cc3)c3ccc(-c4ccc5cc6c(cc5c4)oc4ccccc46)cc3)cccc21 ; CC1(C)c2ccccc2-c2c(N(c3ccccc3)c3ccc(-c4ccc5cc6c(cc5c4)oc4ccccc46)cc3)cccc21]

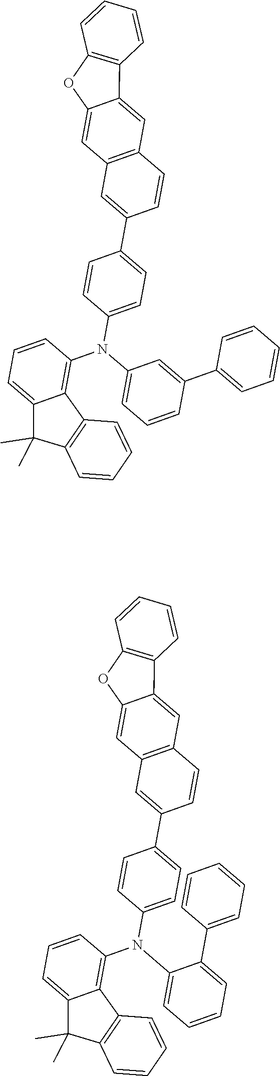 [SMILES: CC1(C)c2ccccc2-c2c(N(c3ccc(-c4ccc5cc6c(cc5c4)oc4ccccc46)cc3)c3cccc(-c4ccccc4)c3)cccc21 ; CC1(C)c2ccccc2-c2c(N(c3ccc(-c4ccc5cc6c(cc5c4)oc4ccccc46)cc3)c3ccccc3-c3ccccc3)cccc21]

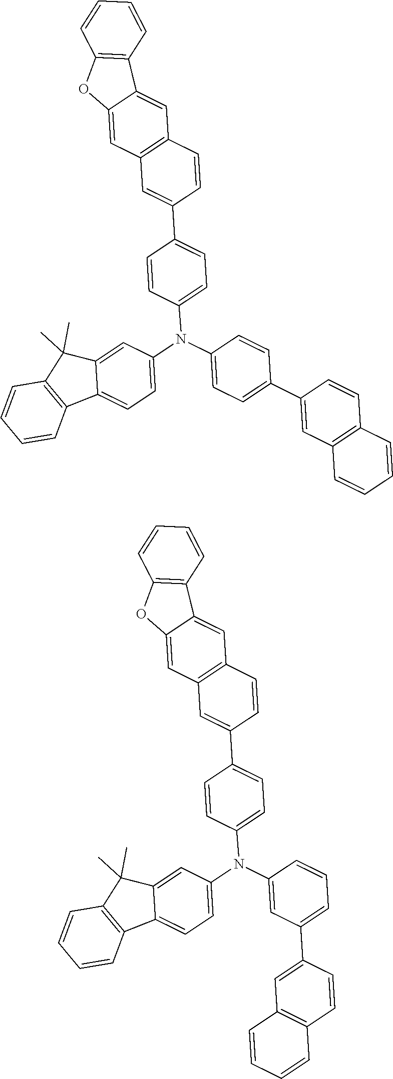 [SMILES: CC1(C)c2ccccc2-c2ccc(N(c3ccc(-c4ccc5ccccc5c4)cc3)c3ccc(-c4ccc5cc6c(cc5c4)oc4ccccc46)cc3)cc21 ; CC1(C)c2ccccc2-c2ccc(N(c3ccc(-c4ccc5cc6c(cc5c4)oc4ccccc46)cc3)c3cccc(-c4ccc5ccccc5c4)c3)cc21]

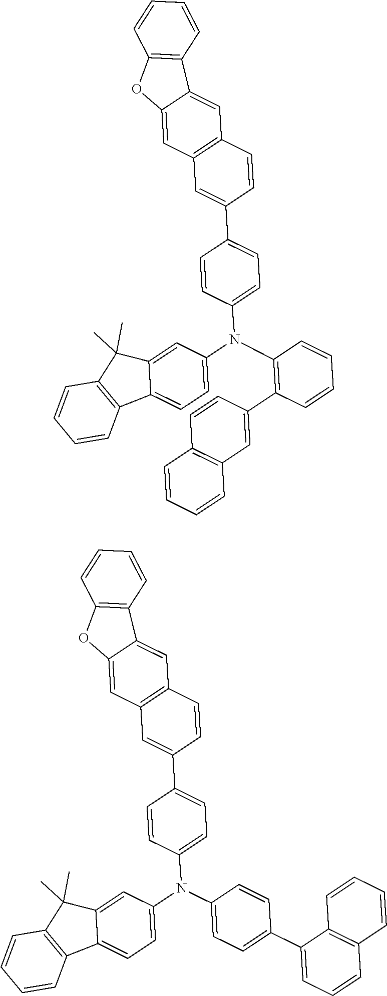 [SMILES: CC1(C)c2ccccc2-c2ccc(N(c3ccc(-c4ccc5cc6c(cc5c4)oc4ccccc46)cc3)c3ccccc3-c3ccc4ccccc4c3)cc21 ; CC1(C)c2ccccc2-c2ccc(N(c3ccc(-c4ccc5cc6c(cc5c4)oc4ccccc46)cc3)c3ccc(-c4cccc5ccccc45)cc3)cc21]

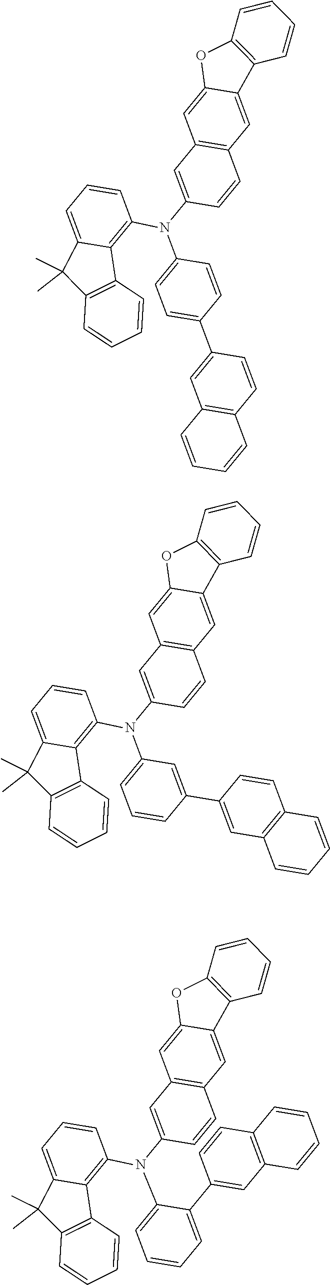

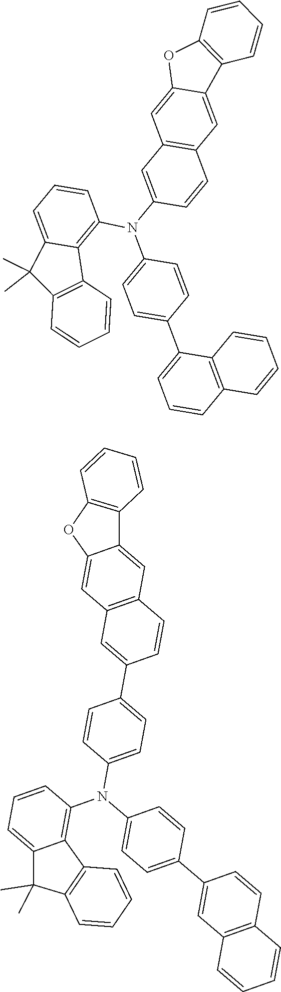 [SMILES: CC1(C)c2ccccc2-c2c(N(c3ccc(-c4cccc5ccccc45)cc3)c3ccc4cc5c(cc4c3)oc3ccccc35)cccc21 ; CC1(C)c2ccccc2-c2c(N(c3ccc(-c4ccc5ccccc5c4)cc3)c3ccc(-c4ccc5cc6c(cc5c4)oc4ccccc46)cc3)cccc21]

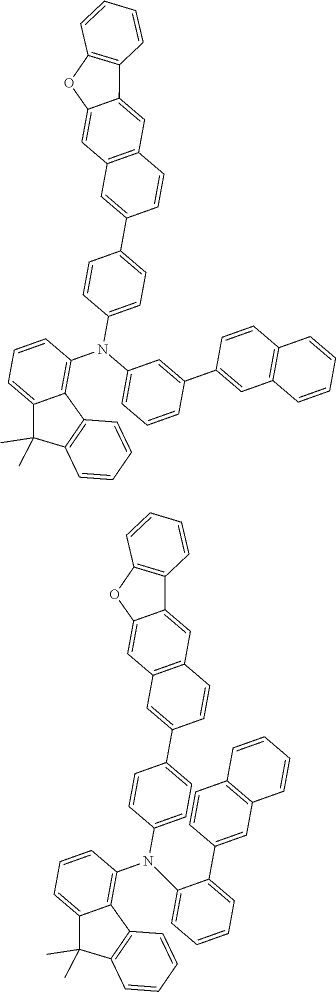 [SMILES: CC1(C)c2ccccc2-c2c(N(c3ccc(-c4ccc5cc6c(cc5c4)oc4ccccc46)cc3)c3cccc(-c4ccc5ccccc5c4)c3)cccc21 ; CC1(C)c2ccccc2-c2c(N(c3ccc(-c4ccc5cc6c(cc5c4)oc4ccccc46)cc3)c3ccccc3-c3ccc4ccccc4c3)cccc21]

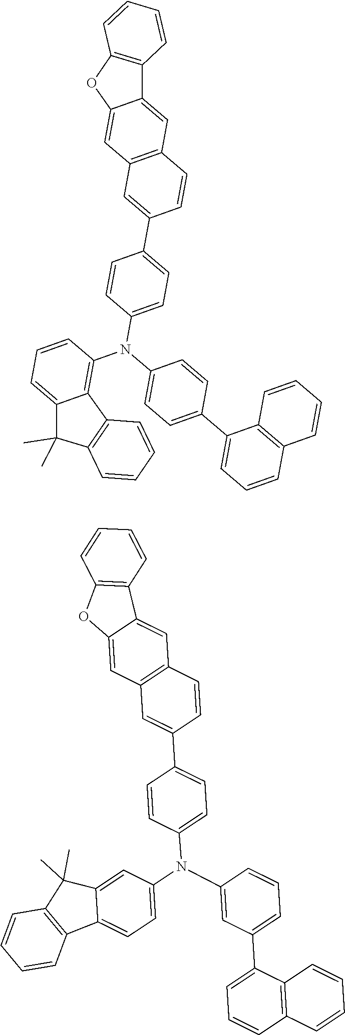 [SMILES: CC1(C)c2ccccc2-c2c(N(c3ccc(-c4ccc5cc6c(cc5c4)oc4ccccc46)cc3)c3ccc(-c4cccc5ccccc45)cc3)cccc21 ; CC1(C)c2ccccc2-c2ccc(N(c3ccc(-c4ccc5cc6c(cc5c4)oc4ccccc46)cc3)c3cccc(-c4cccc5ccccc45)c3)cc21]

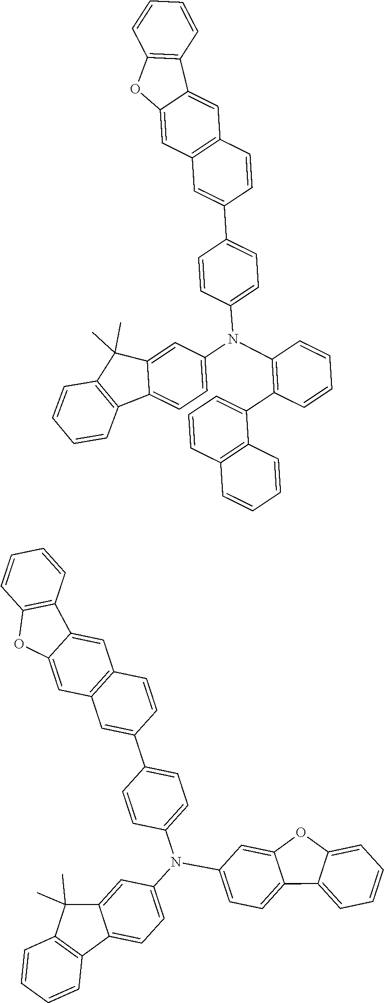 [SMILES: CC1(C)c2ccccc2-c2ccc(N(c3ccc(-c4ccc5cc6c(cc5c4)oc4ccccc46)cc3)c3ccccc3-c3cccc4ccccc34)cc21 ; CC1(C)c2ccccc2-c2ccc(N(c3ccc(-c4ccc5cc6c(cc5c4)oc4ccccc46)cc3)c3ccc4c(c3)oc3ccccc34)cc21]

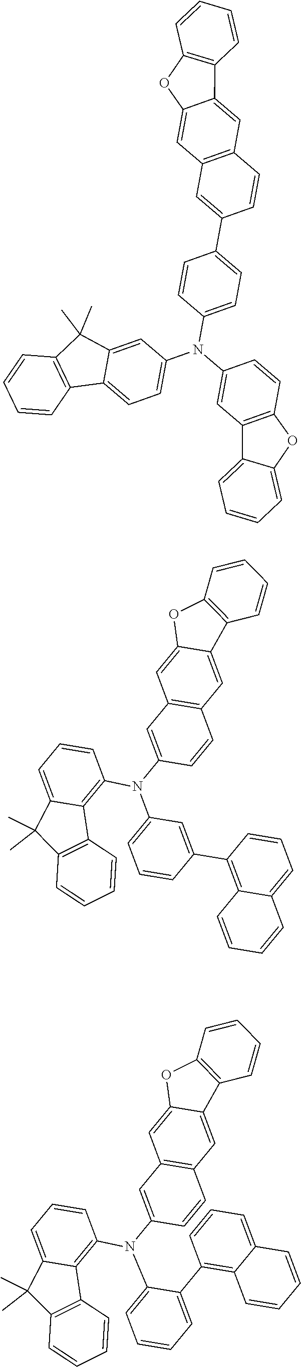

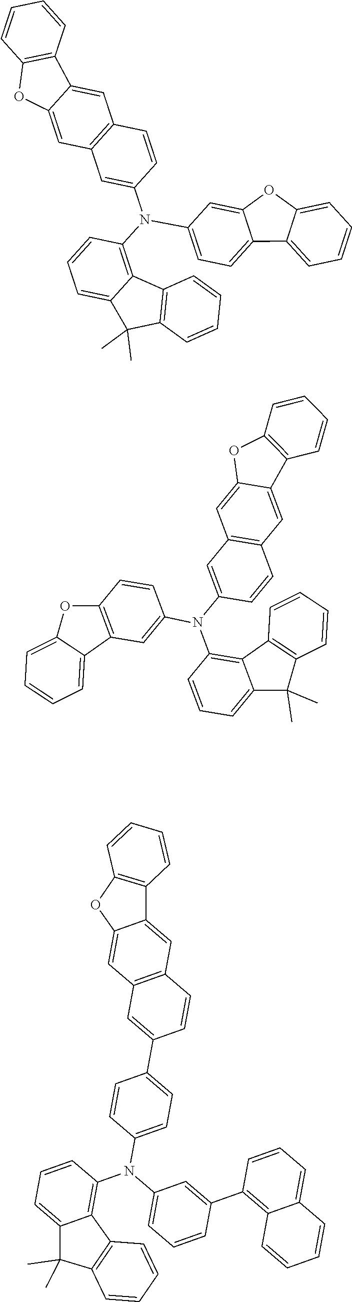 [SMILES: CC1(C)c2ccccc2-c2c(N(c3ccc4cc5c(cc4c3)oc3ccccc35)c3ccc4c(c3)oc3ccccc34)cccc21 ; CC1(C)c2ccccc2-c2c(N(c3ccc4cc5c(cc4c3)oc3ccccc35)c3ccc4oc5ccccc5c4c3)cccc21 ; CC1(C)c2ccccc2-c2c(N(c3ccc(-c4ccc5cc6c(cc5c4)oc4ccccc46)cc3)c3cccc(-c4cccc5ccccc45)c3)cccc21]

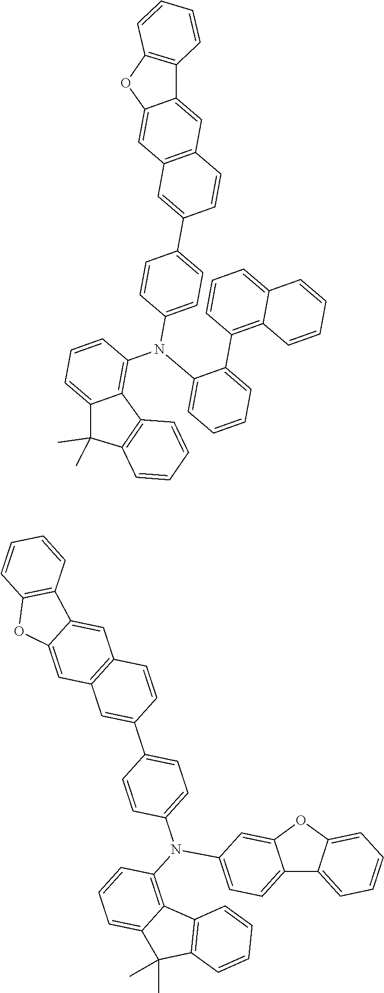 [SMILES: CC1(C)c2ccccc2-c2c(N(c3ccc(-c4ccc5cc6c(cc5c4)oc4ccccc46)cc3)c3ccccc3-c3cccc4ccccc34)cccc21 ; CC1(C)c2ccccc2-c2c(N(c3ccc(-c4ccc5cc6c(cc5c4)oc4ccccc46)cc3)c3ccc4c(c3)oc3ccccc34)cccc21]

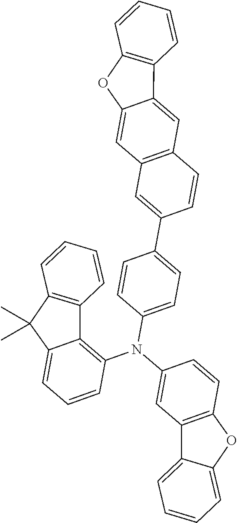 [SMILES: CC1(C)c2ccccc2-c2c(N(c3ccc(-c4ccc5cc6c(cc5c4)oc4ccccc46)cc3)c3ccc4oc5ccccc5c4c3)cccc21]

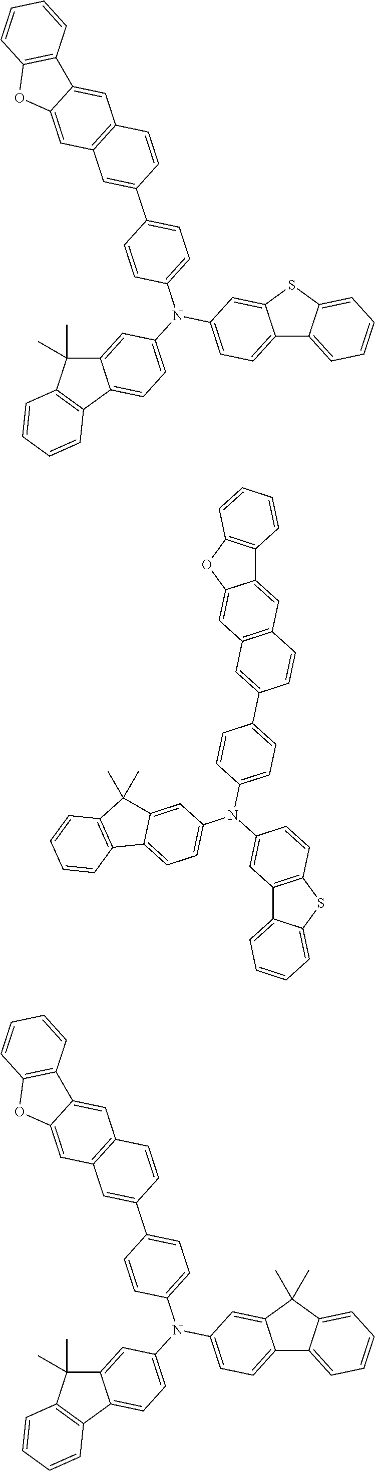 [SMILES: CC1(C)c2ccccc2-c2ccc(N(c3ccc(-c4ccc5cc6c(cc5c4)oc4ccccc46)cc3)c3ccc4c(c3)sc3ccccc34)cc21 ; CC1(C)c2ccccc2-c2ccc(N(c3ccc(-c4ccc5cc6c(cc5c4)oc4ccccc46)cc3)c3ccc4sc5ccccc5c4c3)cc21 ; CC1(C)c2ccccc2-c2ccc(N(c3ccc(-c4ccc5cc6c(cc5c4)oc4ccccc46)cc3)c3ccc4c(c3)C(C)(C)c3ccccc3-4)cc21]

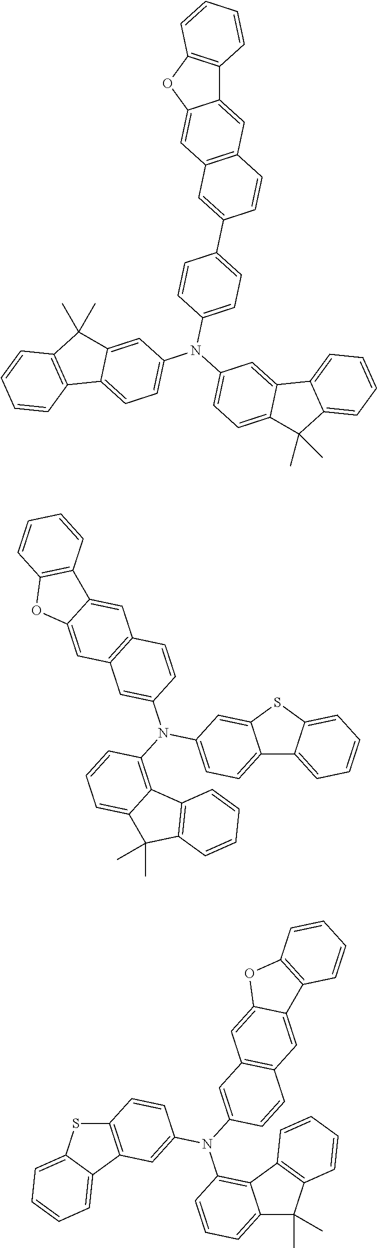 [SMILES: CC1(C)c2ccccc2-c2cc(N(c3ccc(-c4ccc5cc6c(cc5c4)oc4ccccc46)cc3)c3ccc4c(c3)C(C)(C)c3ccccc3-4)ccc21 ; CC1(C)c2ccccc2-c2c(N(c3ccc4cc5c(cc4c3)oc3ccccc35)c3ccc4c(c3)sc3ccccc34)cccc21 ; CC1(C)c2ccccc2-c2c(N(c3ccc4cc5c(cc4c3)oc3ccccc35)c3ccc4sc5ccccc5c4c3)cccc21]

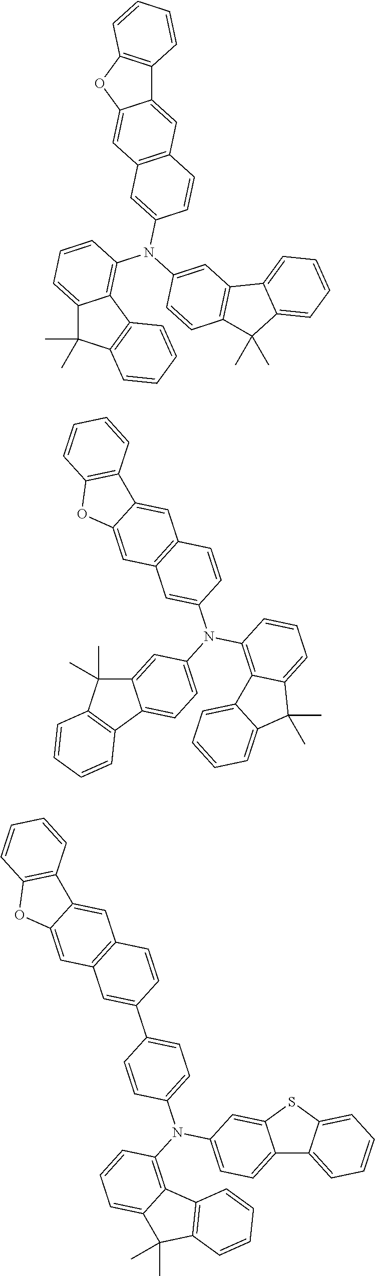

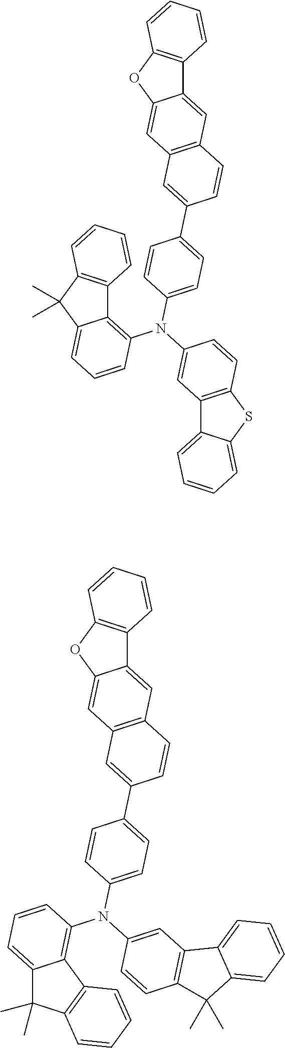 [SMILES: CC1(C)c2ccccc2-c2c(N(c3ccc(-c4ccc5cc6c(cc5c4)oc4ccccc46)cc3)c3ccc4sc5ccccc5c4c3)cccc21 ; CC1(C)c2ccccc2-c2cc(N(c3ccc(-c4ccc5cc6c(cc5c4)oc4ccccc46)cc3)c3cccc4c3-c3ccccc3C4(C)C)ccc21]

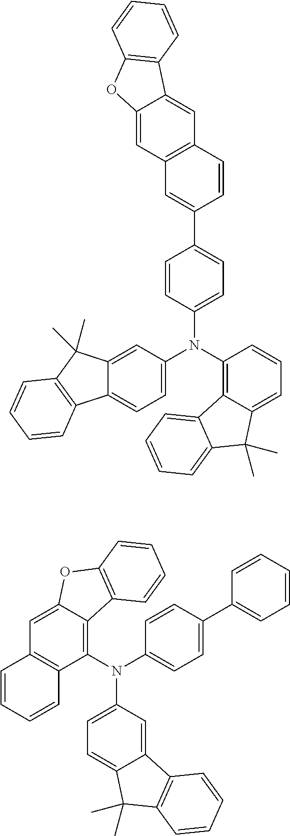 [SMILES: CC1(C)c2ccccc2-c2ccc(N(c3ccc(-c4ccc5cc6c(cc5c4)oc4ccccc46)cc3)c3cccc4c3-c3ccccc3C4(C)C)cc21 ; CC1(C)c2ccccc2-c2cc(N(c3ccc(-c4ccccc4)cc3)c3c4ccccc4cc4oc5ccccc5c34)ccc21]

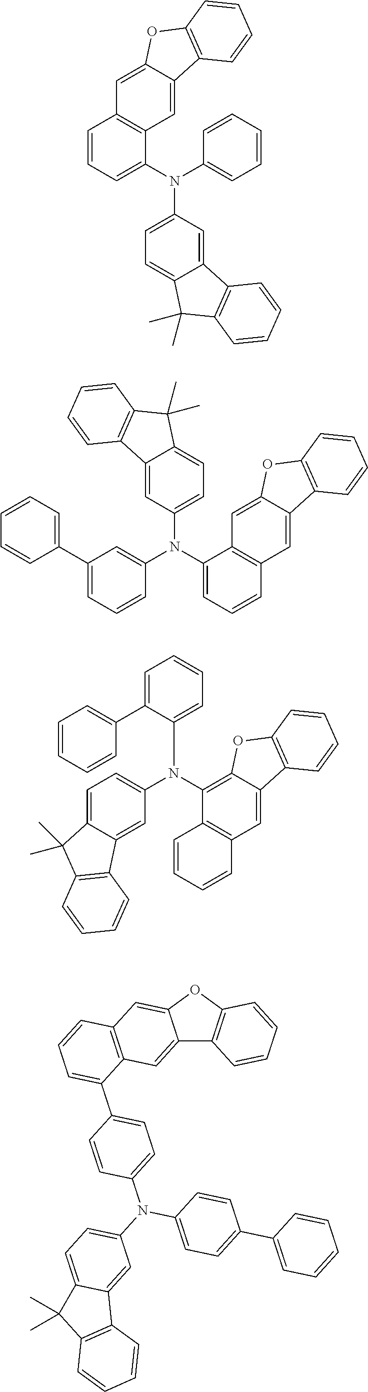

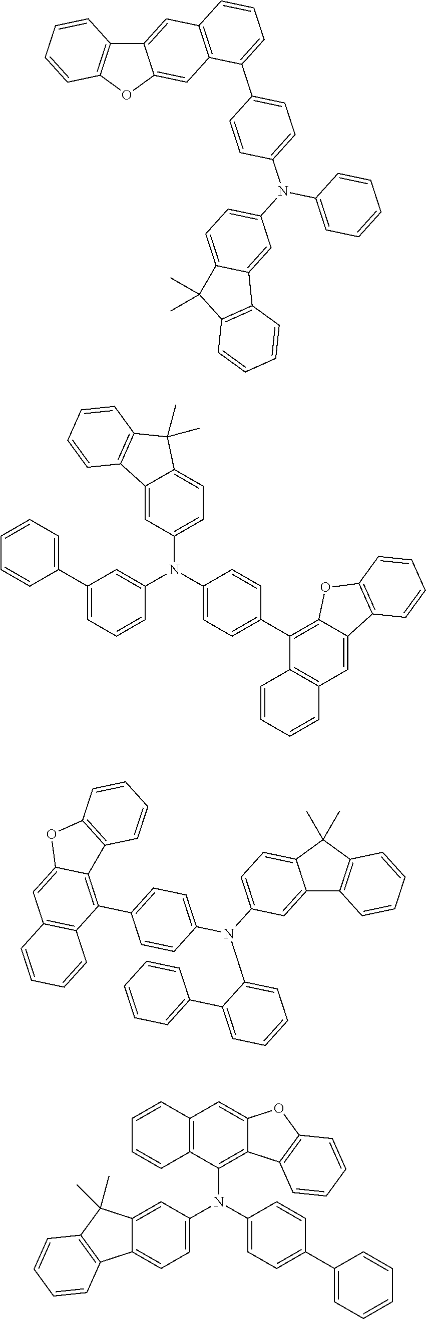 [SMILES: CC1(C)c2ccccc2-c2cc(N(c3ccccc3)c3ccc(-c4cccc5cc6c(cc45)oc4ccccc46)cc3)ccc21 ; CC1(C)c2ccccc2-c2cc(N(c3ccc(-c4c5ccccc5cc5c4oc4ccccc45)cc3)c3cccc(-c4ccccc4)c3)ccc21 ; CC1(C)c2ccccc2-c2cc(N(c3ccc(-c4c5ccccc5cc5oc6ccccc6c45)cc3)c3ccccc3-c3ccccc3)ccc21 ; CC1(C)c2ccccc2-c2ccc(N(c3ccc(-c4ccccc4)cc3)c3c4ccccc4cc4oc5ccccc5c34)cc21]

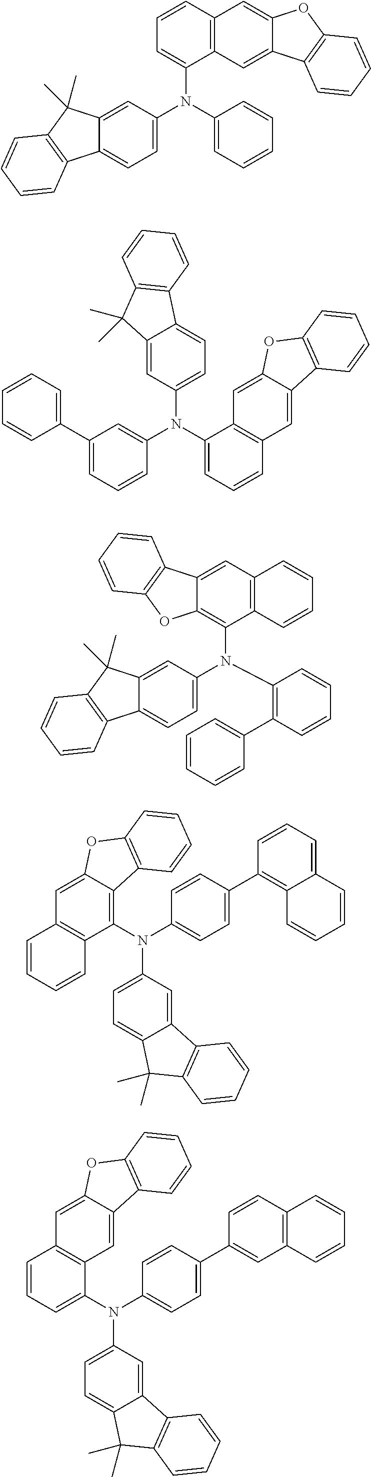

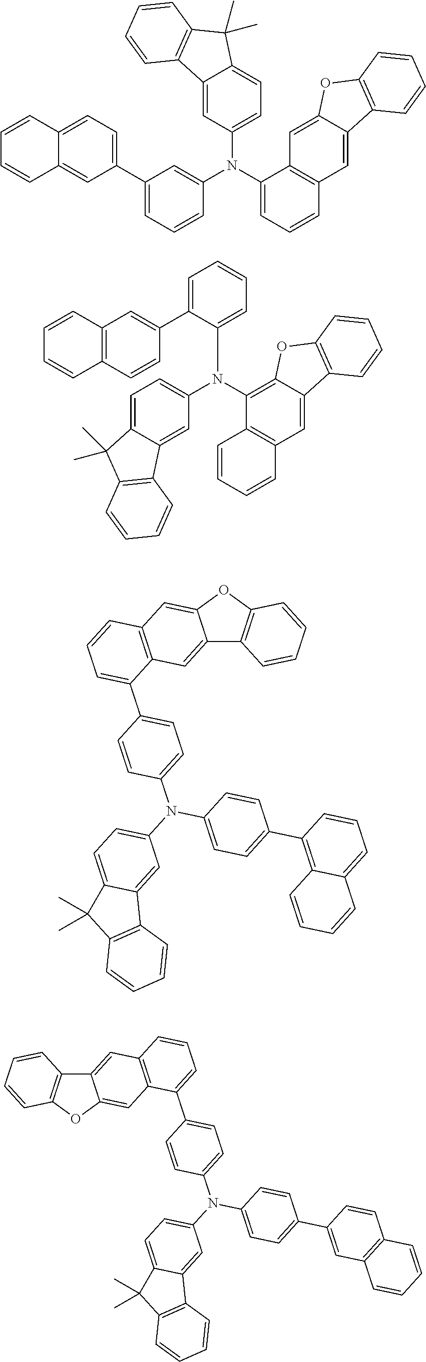 [SMILES: CC1(C)c2ccccc2-c2cc(N(c3cccc(-c4ccc5ccccc5c4)c3)c3cccc4cc5c(cc34)oc3ccccc35)ccc21 ; CC1(C)c2ccccc2-c2cc(N(c3ccccc3-c3ccc4ccccc4c3)c3c4ccccc4cc4c3oc3ccccc34)ccc21 ; CC1(C)c2ccccc2-c2cc(N(c3ccc(-c4cccc5ccccc45)cc3)c3ccc(-c4cccc5cc6oc7ccccc7c6cc45)cc3)ccc21 ; CC1(C)c2ccccc2-c2cc(N(c3ccc(-c4ccc5ccccc5c4)cc3)c3ccc(-c4cccc5cc6c(cc45)oc4ccccc46)cc3)ccc21]

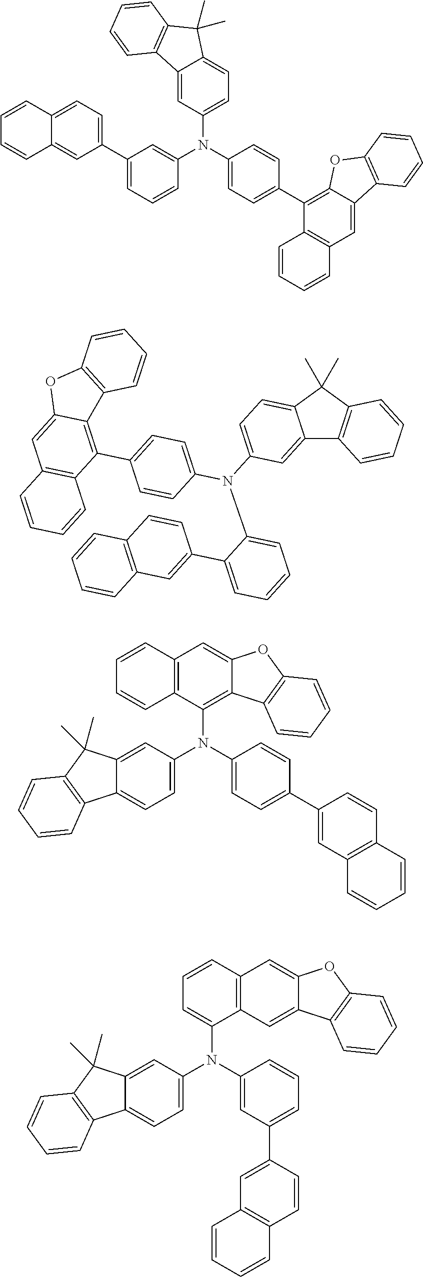 [SMILES: CC1(C)c2ccccc2-c2cc(N(c3ccc(-c4c5ccccc5cc5c4oc4ccccc45)cc3)c3cccc(-c4ccc5ccccc5c4)c3)ccc21 ; CC1(C)c2ccccc2-c2cc(N(c3ccc(-c4c5ccccc5cc5oc6ccccc6c45)cc3)c3ccccc3-c3ccc4ccccc4c3)ccc21 ; CC1(C)c2ccccc2-c2ccc(N(c3ccc(-c4ccc5ccccc5c4)cc3)c3c4ccccc4cc4oc5ccccc5c34)cc21 ; CC1(C)c2ccccc2-c2ccc(N(c3cccc(-c4ccc5ccccc5c4)c3)c3cccc4cc5oc6ccccc6c5cc34)cc21]

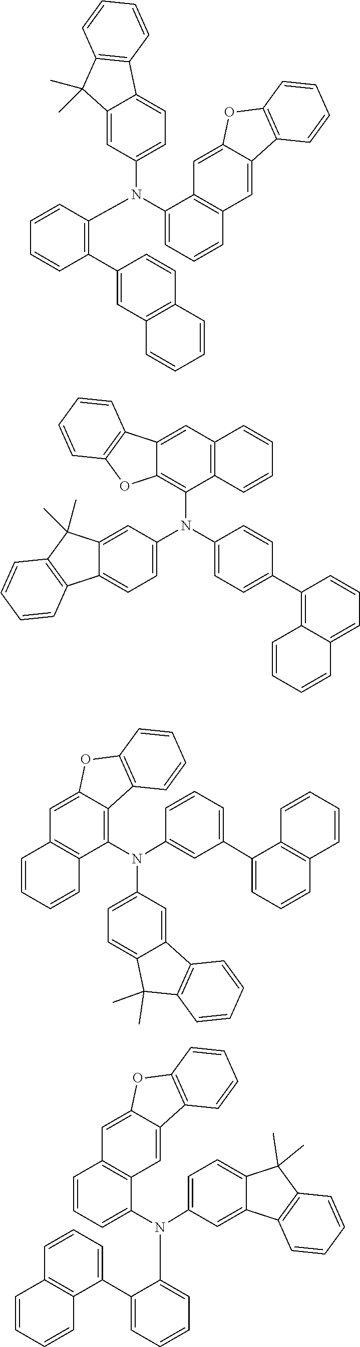

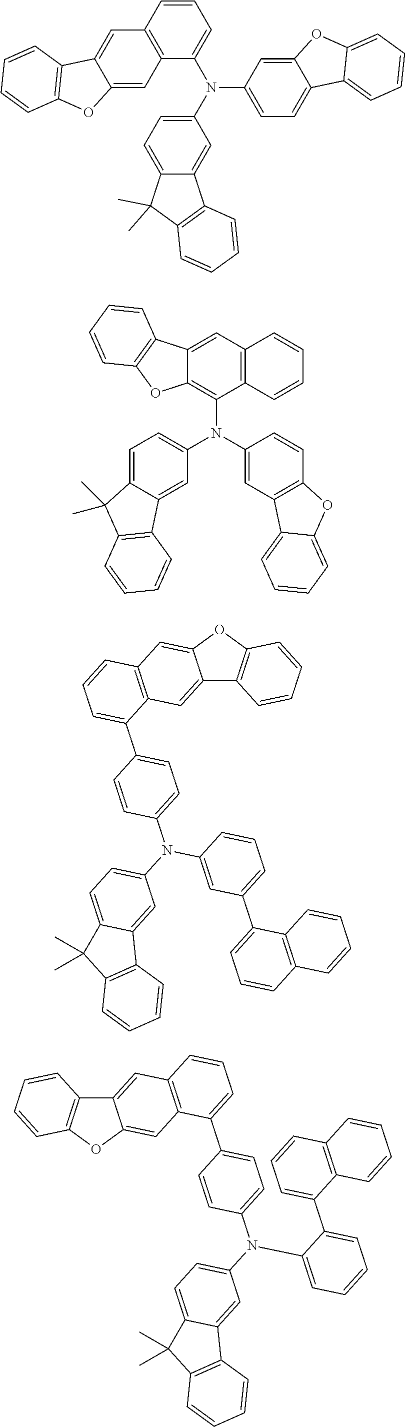

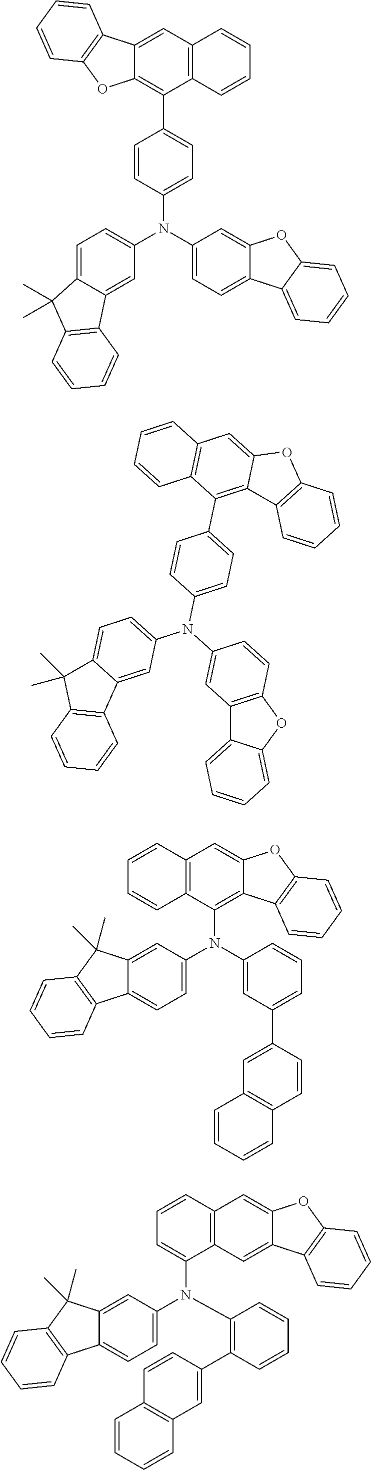

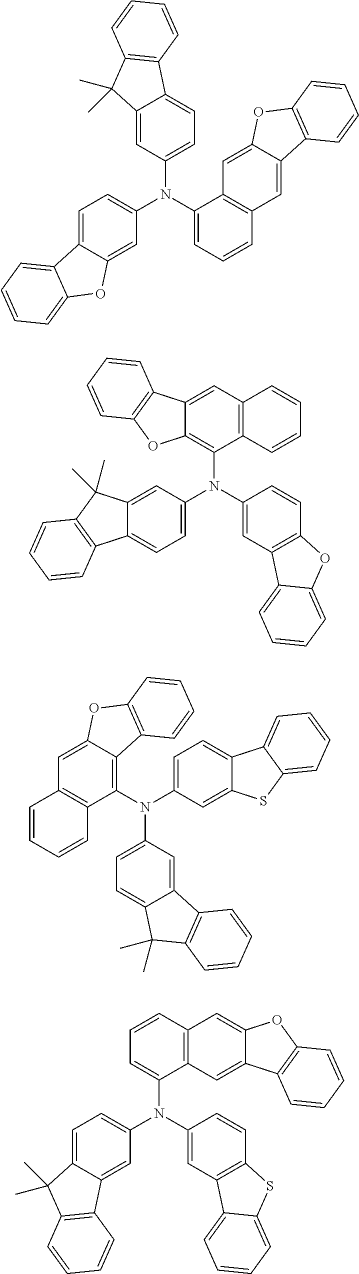

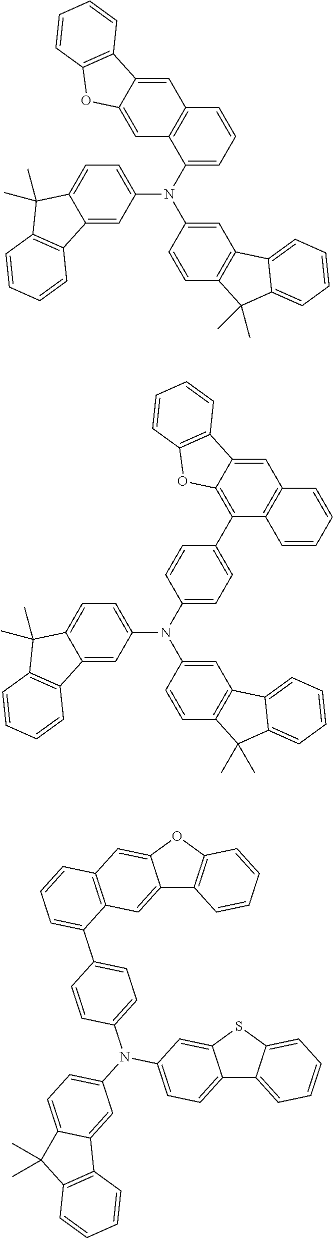 [SMILES: CC1(C)c2ccccc2-c2cc(N(c3ccc4c(c3)-c3ccccc3C4(C)C)c3cccc4cc5c(cc34)oc3ccccc35)ccc21 ; CC1(C)c2ccccc2-c2cc(N(c3ccc(-c4c5ccccc5cc5c4oc4ccccc45)cc3)c3ccc4c(c3)-c3ccccc3C4(C)C)ccc21 ; CC1(C)c2ccccc2-c2cc(N(c3ccc(-c4cccc5cc6oc7ccccc7c6cc45)cc3)c3ccc4c(c3)sc3ccccc34)ccc21]

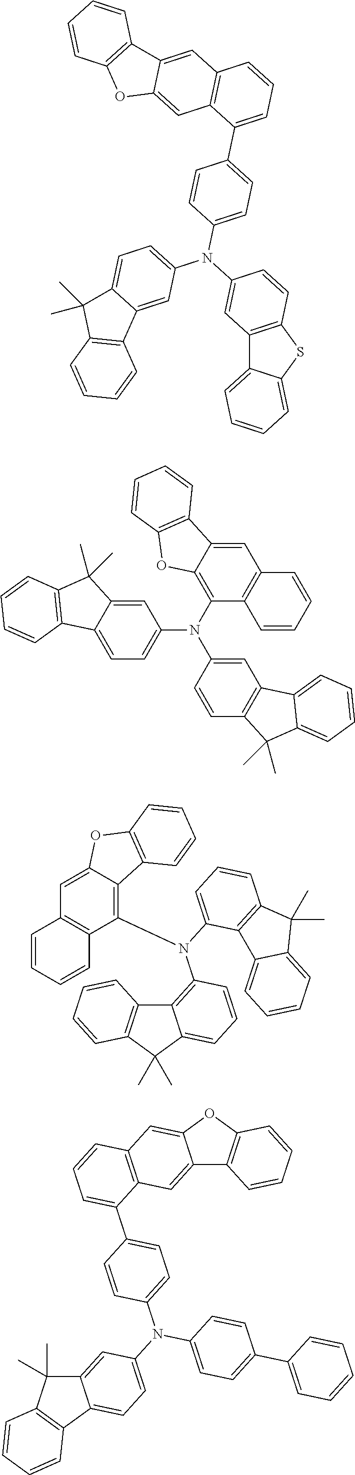

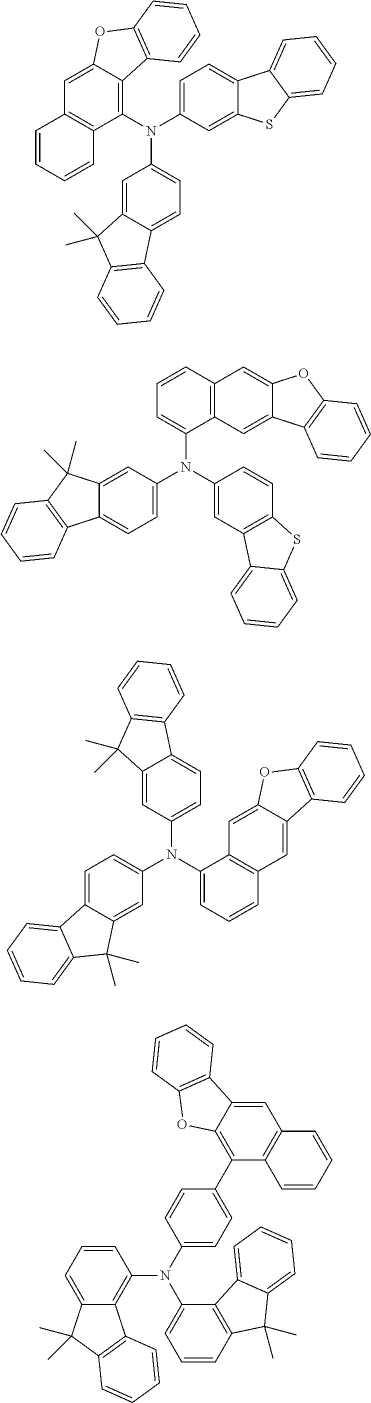 [SMILES: CC1(C)c2ccccc2-c2ccc(N(c3ccc4c(c3)sc3ccccc34)c3c4ccccc4cc4oc5ccccc5c34)cc21 ; CC1(C)c2ccccc2-c2ccc(N(c3ccc4sc5ccccc5c4c3)c3cccc4cc5oc6ccccc6c5cc34)cc21 ; CC1(C)c2ccccc2-c2ccc(N(c3ccc4c(c3)C(C)(C)c3ccccc3-4)c3cccc4cc5c(cc34)oc3ccccc35)cc21 ; CC1(C)c2ccccc2-c2c(N(c3ccc(-c4c5ccccc5cc5c4oc4ccccc45)cc3)c3cccc4c3-c3ccccc3C4(C)C)cccc21]

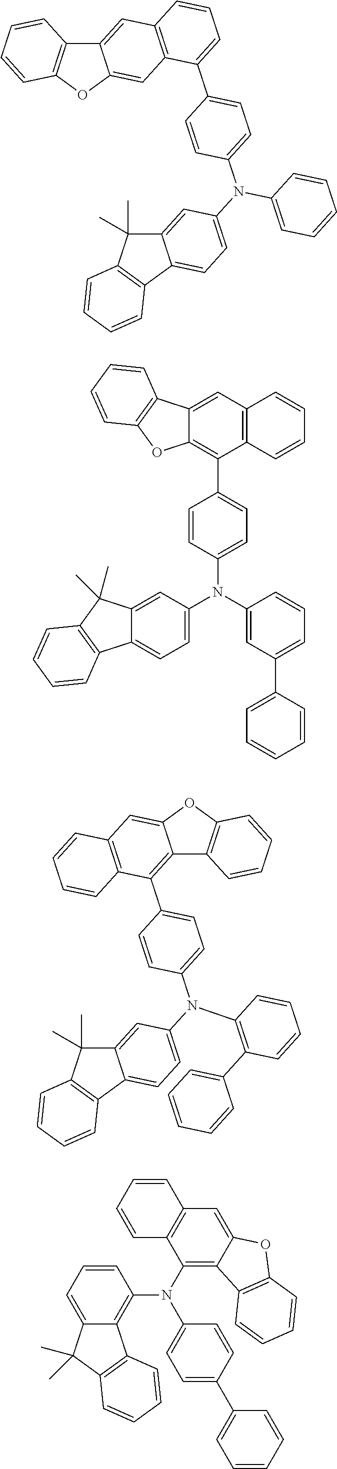

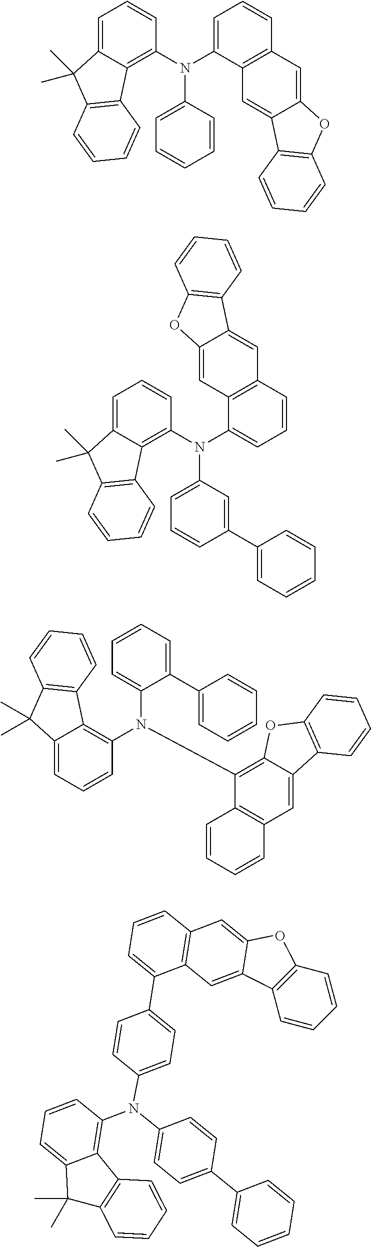 [SMILES: CC1(C)c2ccccc2-c2c(N(c3ccccc3)c3cccc4cc5oc6ccccc6c5cc34)cccc21 ; CC1(C)c2ccccc2-c2c(N(c3cccc(-c4ccccc4)c3)c3cccc4cc5c(cc34)oc3ccccc35)cccc21 ; CC1(C)c2ccccc2-c2c(N(c3ccccc3-c3ccccc3)c3c4ccccc4cc4c3oc3ccccc34)cccc21 ; CC1(C)c2ccccc2-c2c(N(c3ccc(-c4ccccc4)cc3)c3ccc(-c4cccc5cc6oc7ccccc7c6cc45)cc3)cccc21]

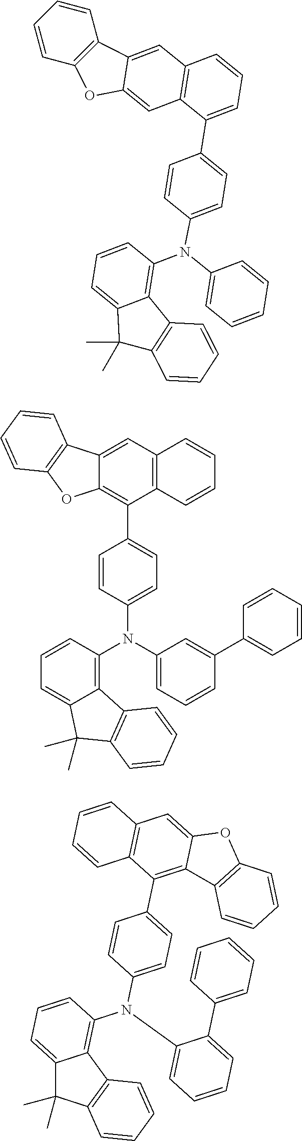

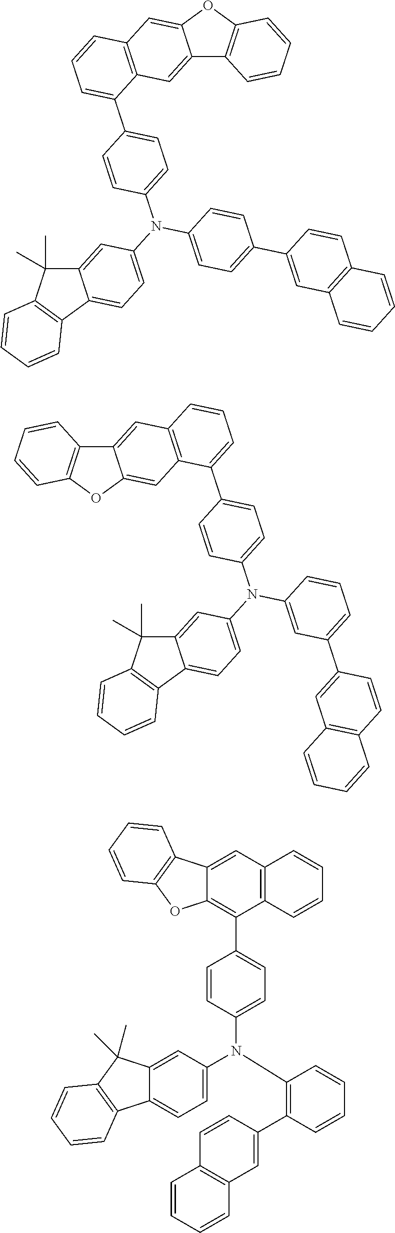 [SMILES: CC1(C)c2ccccc2-c2ccc(N(c3ccc(-c4ccc5ccccc5c4)cc3)c3ccc(-c4cccc5cc6oc7ccccc7c6cc45)cc3)cc21 ; CC1(C)c2ccccc2-c2ccc(N(c3ccc(-c4cccc5cc6c(cc45)oc4ccccc46)cc3)c3cccc(-c4ccc5ccccc5c4)c3)cc21 ; CC1(C)c2ccccc2-c2ccc(N(c3ccc(-c4c5ccccc5cc5c4oc4ccccc45)cc3)c3ccccc3-c3ccc4ccccc4c3)cc21]

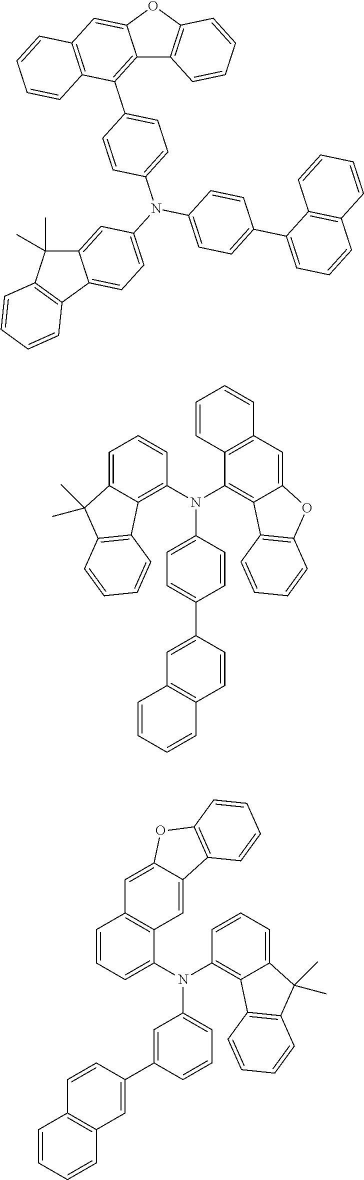 [SMILES: CC1(C)c2ccccc2-c2ccc(N(c3ccc(-c4cccc5ccccc45)cc3)c3ccc(-c4c5ccccc5cc5oc6ccccc6c45)cc3)cc21 ; CC1(C)c2ccccc2-c2c(N(c3ccc(-c4ccc5ccccc5c4)cc3)c3c4ccccc4cc4oc5ccccc5c34)cccc21 ; CC1(C)c2ccccc2-c2c(N(c3cccc(-c4ccc5ccccc5c4)c3)c3cccc4cc5oc6ccccc6c5cc34)cccc21]

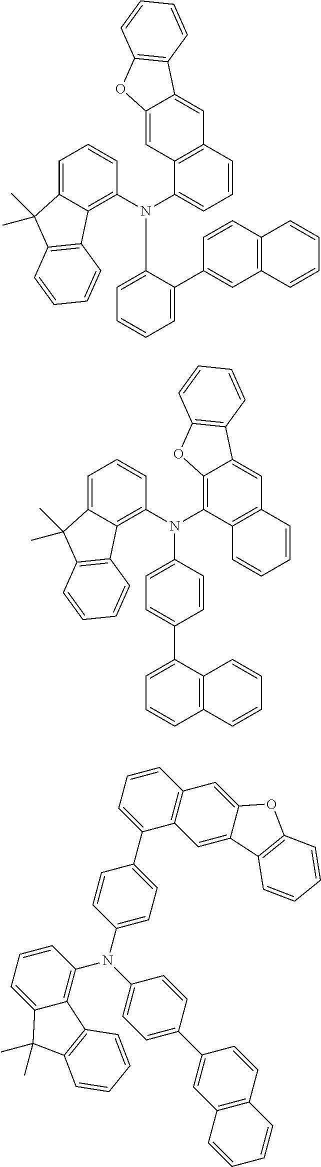

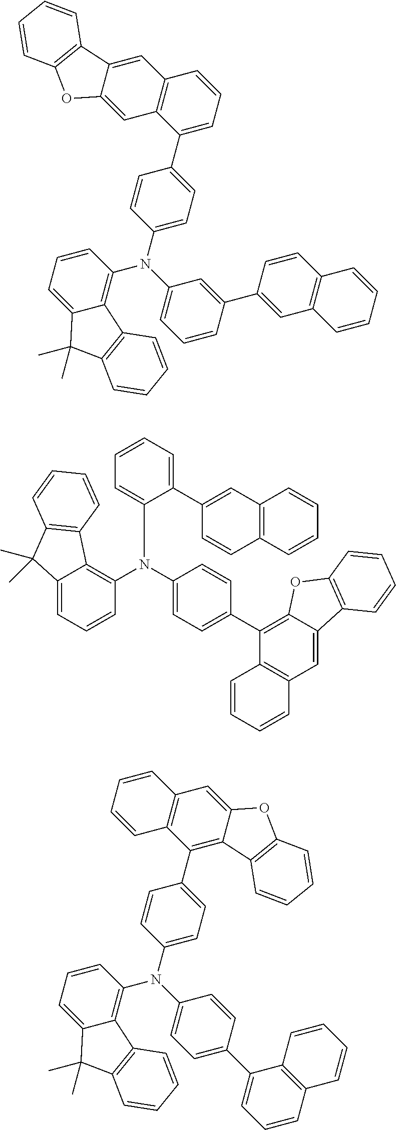

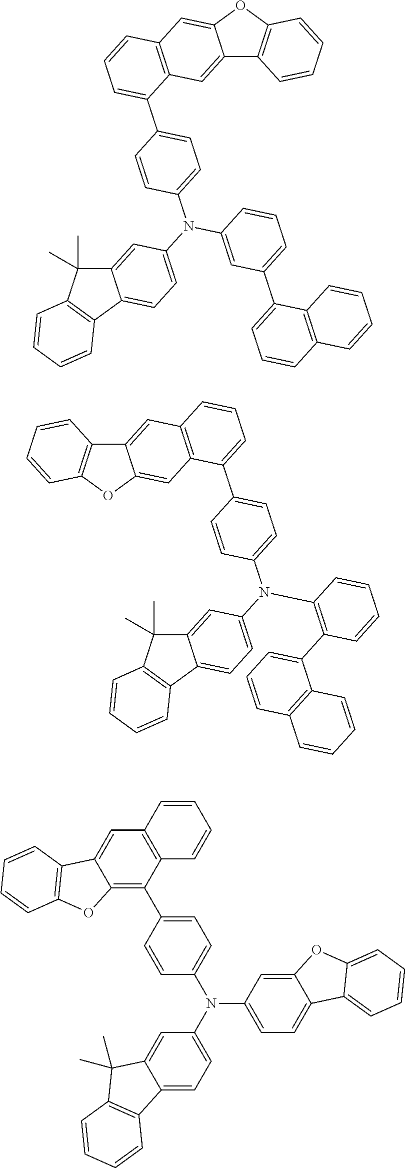 [SMILES: CC1(C)c2ccccc2-c2ccc(N(c3ccc(-c4cccc5cc6oc7ccccc7c6cc45)cc3)c3cccc(-c4cccc5ccccc45)c3)cc21 ; CC1(C)c2ccccc2-c2ccc(N(c3ccc(-c4cccc5cc6c(cc45)oc4ccccc46)cc3)c3ccccc3-c3cccc4ccccc34)cc21 ; CC1(C)c2ccccc2-c2ccc(N(c3ccc(-c4c5ccccc5cc5c4oc4ccccc45)cc3)c3ccc4c(c3)oc3ccccc34)cc21]

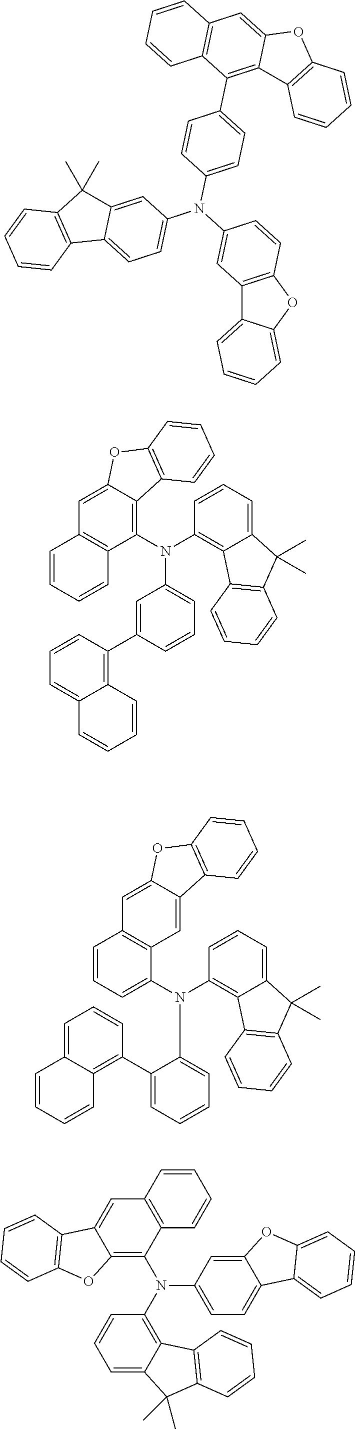 [SMILES: CC1(C)c2ccccc2-c2ccc(N(c3ccc(-c4c5ccccc5cc5oc6ccccc6c45)cc3)c3ccc4oc5ccccc5c4c3)cc21 ; CC1(C)c2ccccc2-c2c(N(c3cccc(-c4cccc5ccccc45)c3)c3c4ccccc4cc4oc5ccccc5c34)cccc21 ; CC1(C)c2ccccc2-c2c(N(c3ccccc3-c3cccc4ccccc34)c3cccc4cc5oc6ccccc6c5cc34)cccc21 ; CC1(C)c2ccccc2-c2c(N(c3ccc4c(c3)oc3ccccc34)c3c4ccccc4cc4c3oc3ccccc34)cccc21]

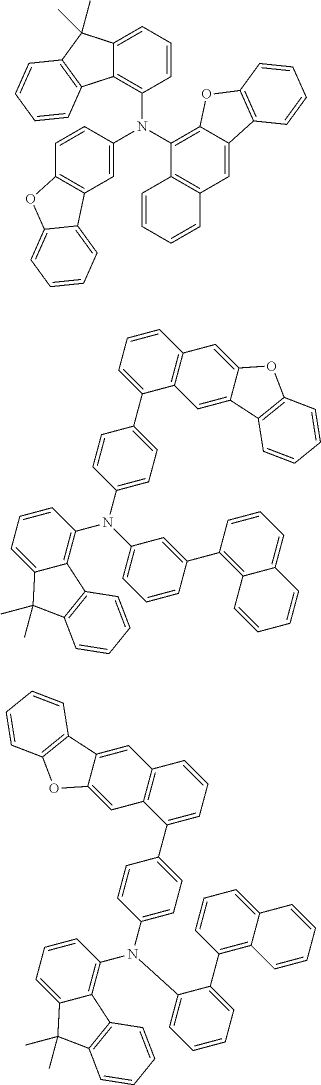 [SMILES: CC1(C)c2ccccc2-c2c(N(c3ccc4oc5ccccc5c4c3)c3c4ccccc4cc4c3oc3ccccc34)cccc21 ; CC1(C)c2ccccc2-c2c(N(c3ccc(-c4cccc5cc6oc7ccccc7c6cc45)cc3)c3cccc(-c4cccc5ccccc45)c3)cccc21 ; CC1(C)c2ccccc2-c2c(N(c3ccc(-c4cccc5cc6c(cc45)oc4ccccc46)cc3)c3ccccc3-c3cccc4ccccc34)cccc21]

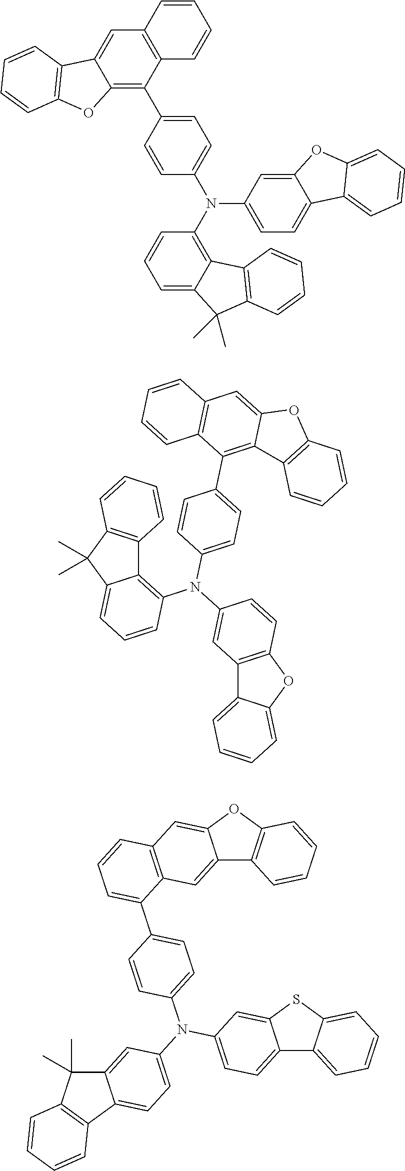

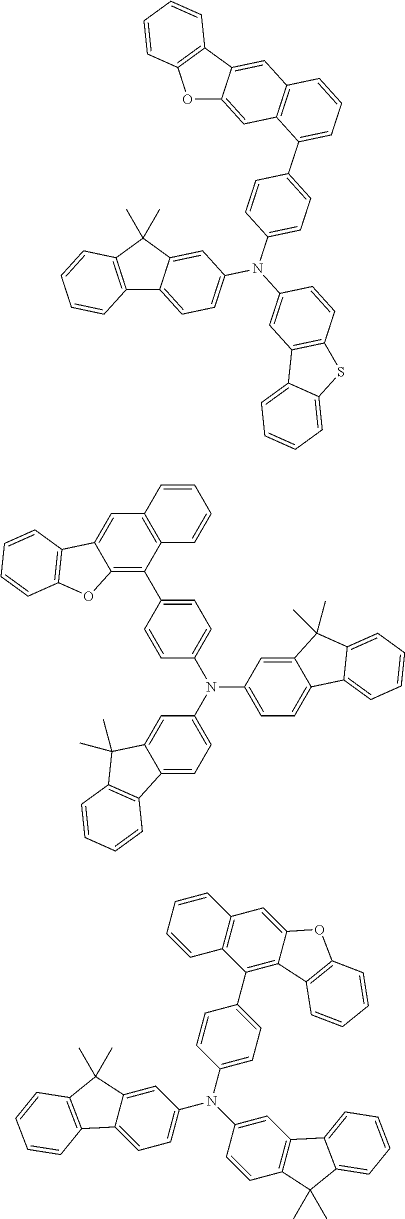 [SMILES: CC1(C)c2ccccc2-c2ccc(N(c3ccc(-c4cccc5cc6c(cc45)oc4ccccc46)cc3)c3ccc4sc5ccccc5c4c3)cc21 ; CC1(C)c2ccccc2-c2ccc(N(c3ccc(-c4c5ccccc5cc5c4oc4ccccc45)cc3)c3ccc4c(c3)C(C)(C)c3ccccc3-4)cc21 ; CC1(C)c2ccccc2-c2cc(N(c3ccc(-c4c5ccccc5cc5oc6ccccc6c45)cc3)c3ccc4c(c3)C(C)(C)c3ccccc3-4)ccc21]

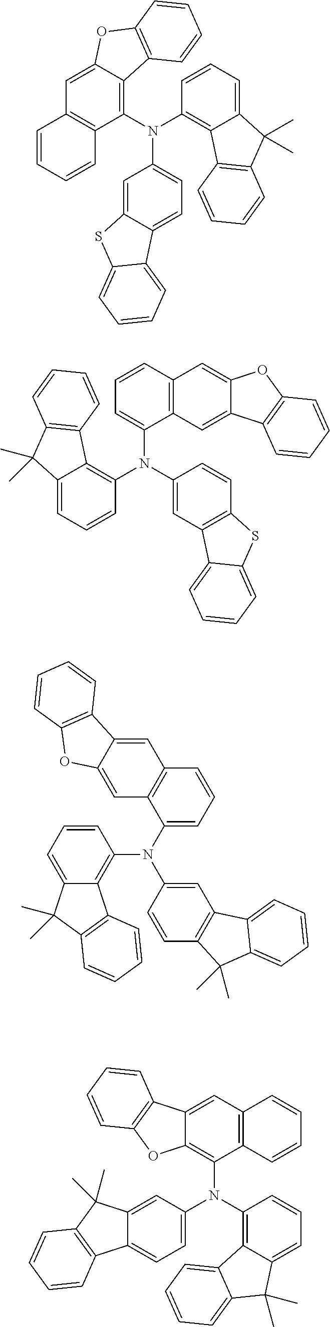

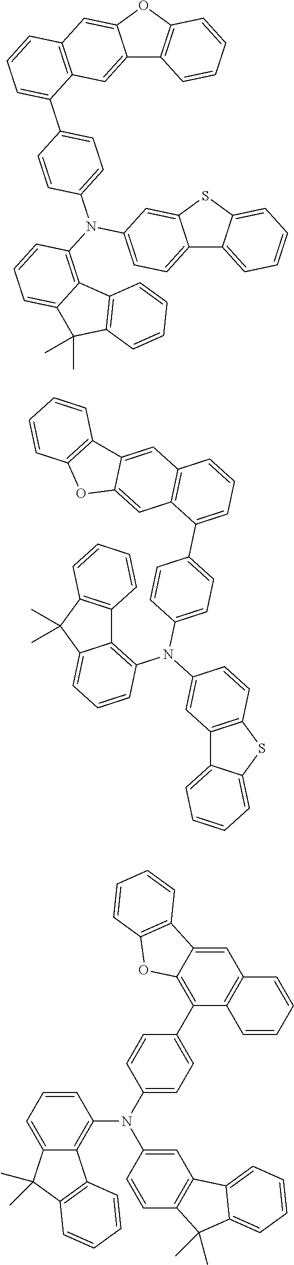

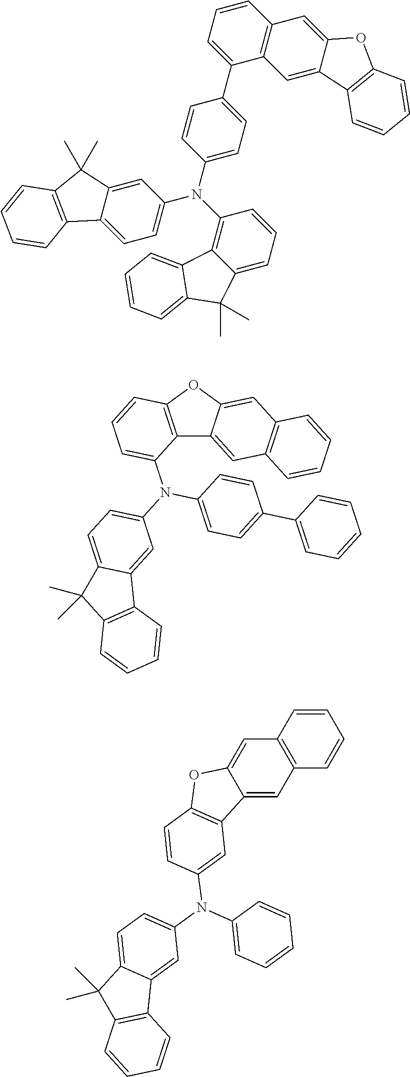 [SMILES: CC1(C)c2ccccc2-c2ccc(N(c3ccc(-c4cccc5cc6oc7ccccc7c6cc45)cc3)c3cccc4c3-c3ccccc3C4(C)C)cc21 ; CC1(C)c2ccccc2-c2cc(N(c3ccc(-c4ccccc4)cc3)c3cccc4oc5cc6ccccc6cc5c34)ccc21 ; CC1(C)c2ccccc2-c2cc(N(c3ccccc3)c3ccc4oc5cc6ccccc6cc5c4c3)ccc21]

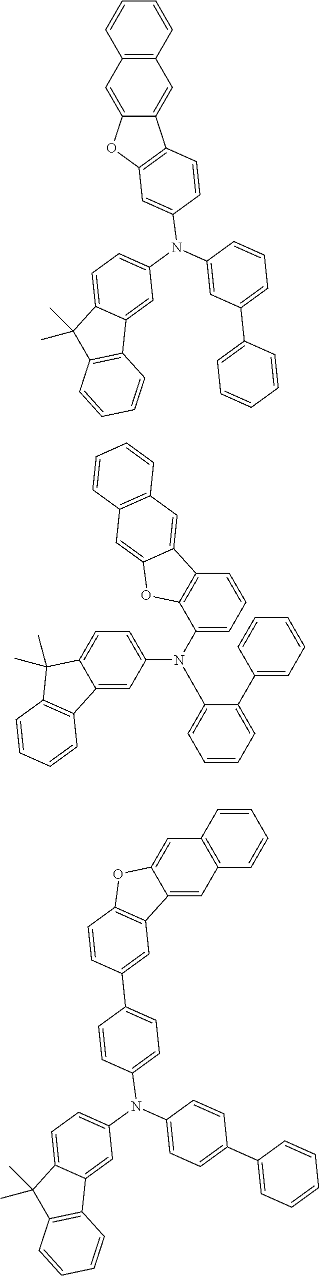

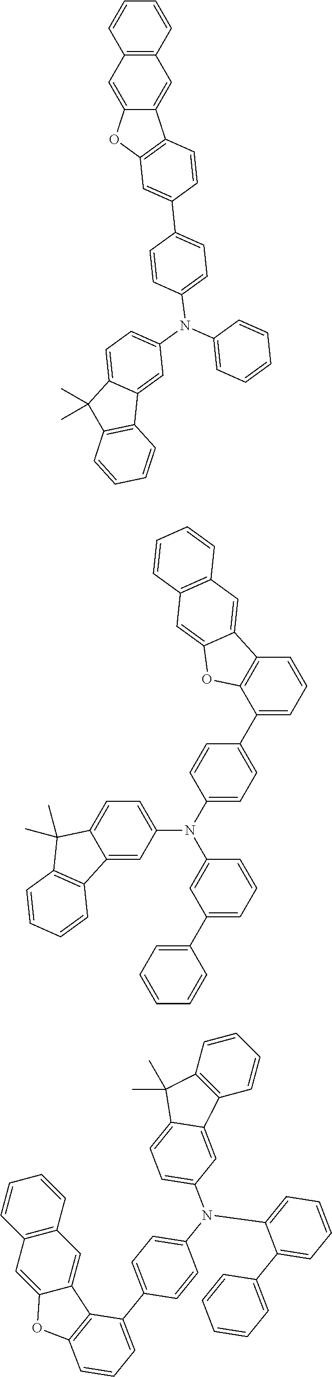

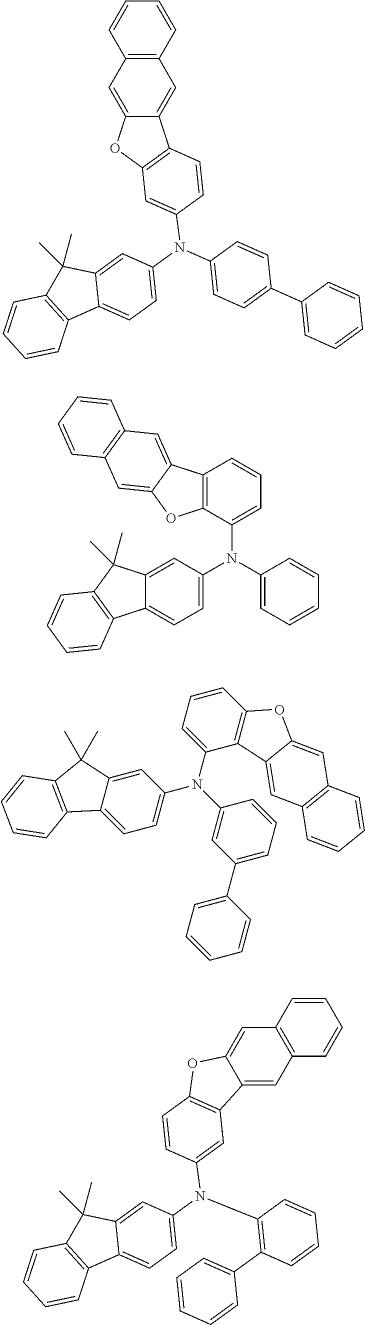 [SMILES: CC1(C)c2ccccc2-c2ccc(N(c3ccc(-c4ccccc4)cc3)c3ccc4c(c3)oc3cc5ccccc5cc34)cc21 ; CC1(C)c2ccccc2-c2ccc(N(c3ccccc3)c3cccc4c3oc3cc5ccccc5cc34)cc21 ; CC1(C)c2ccccc2-c2ccc(N(c3cccc(-c4ccccc4)c3)c3cccc4oc5cc6ccccc6cc5c34)cc21 ; CC1(C)c2ccccc2-c2ccc(N(c3ccc4oc5cc6ccccc6cc5c4c3)c3ccccc3-c3ccccc3)cc21]

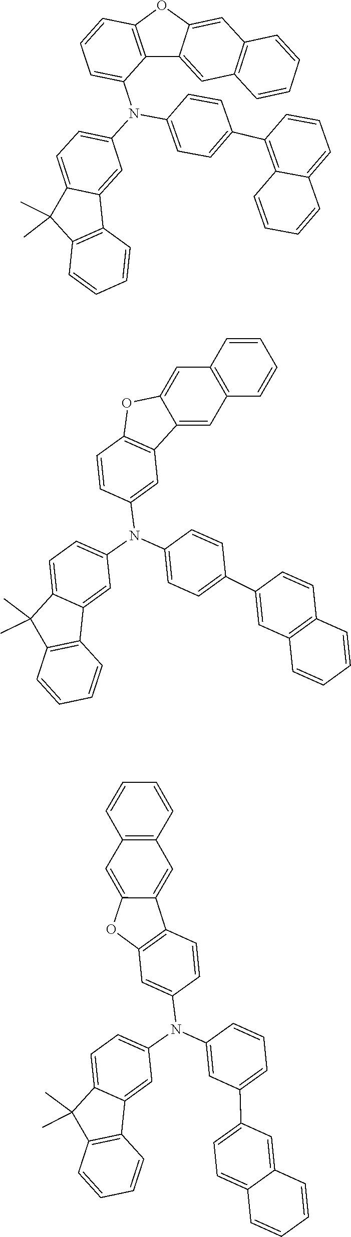 [SMILES: CC1(C)c2ccccc2-c2cc(N(c3ccc(-c4cccc5ccccc45)cc3)c3cccc4oc5cc6ccccc6cc5c34)ccc21 ; CC1(C)c2ccccc2-c2cc(N(c3ccc(-c4ccc5ccccc5c4)cc3)c3ccc4oc5cc6ccccc6cc5c4c3)ccc21 ; CC1(C)c2ccccc2-c2cc(N(c3cccc(-c4ccc5ccccc5c4)c3)c3ccc4c(c3)oc3cc5ccccc5cc34)ccc21]

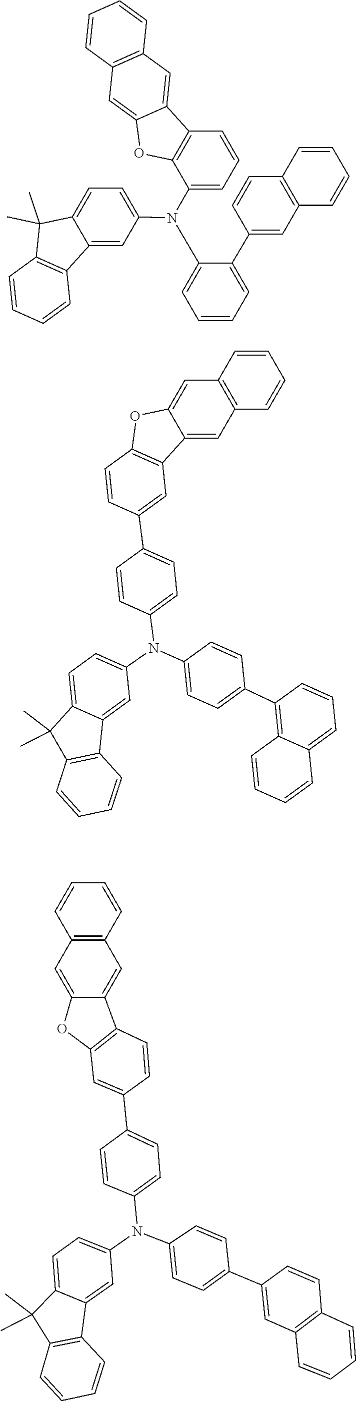

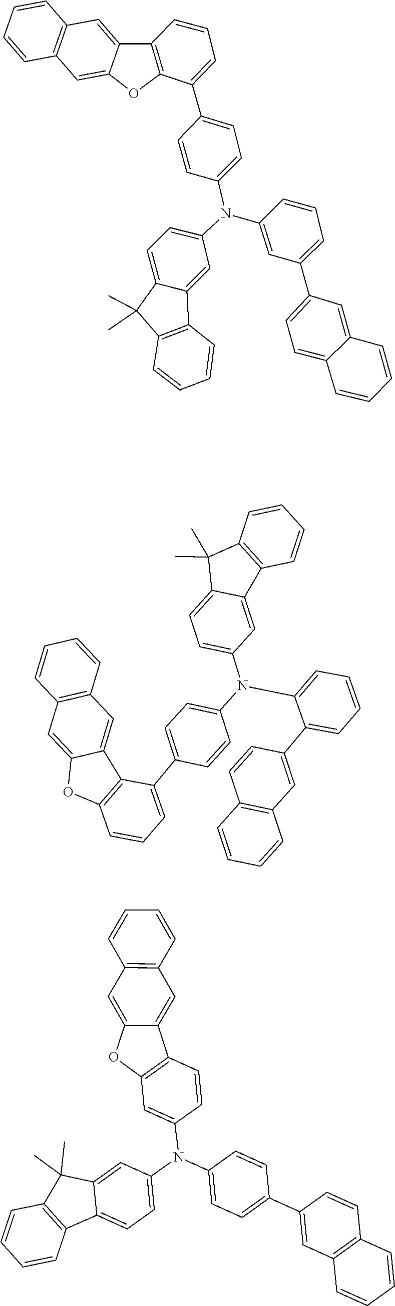 [SMILES: CC1(C)c2ccccc2-c2cc(N(c3ccc(-c4cccc5c4oc4cc6ccccc6cc45)cc3)c3cccc(-c4ccc5ccccc5c4)c3)ccc21 ; CC1(C)c2ccccc2-c2cc(N(c3ccc(-c4cccc5oc6cc7ccccc7cc6c45)cc3)c3ccccc3-c3ccc4ccccc4c3)ccc21 ; CC1(C)c2ccccc2-c2ccc(N(c3ccc(-c4ccc5ccccc5c4)cc3)c3ccc4c(c3)oc3cc5ccccc5cc34)cc21]

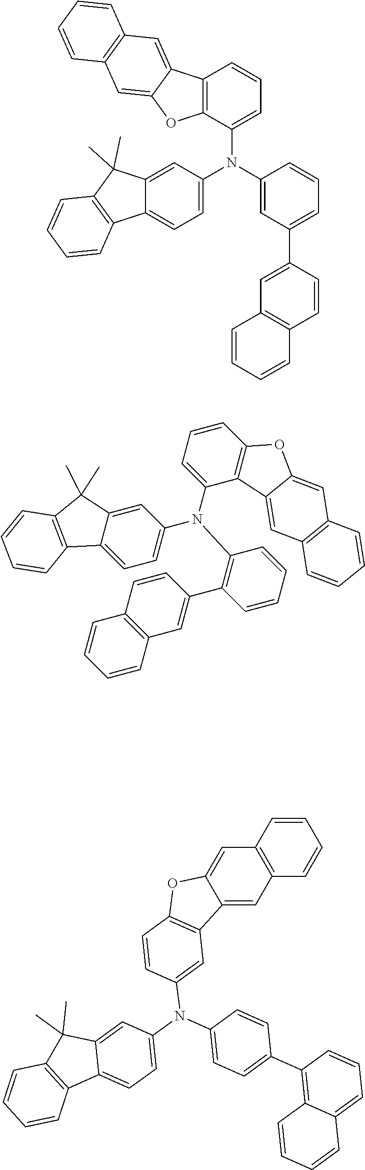 [SMILES: CC1(C)c2ccccc2-c2ccc(N(c3cccc(-c4ccc5ccccc5c4)c3)c3cccc4c3oc3cc5ccccc5cc34)cc21 ; CC1(C)c2ccccc2-c2ccc(N(c3ccccc3-c3ccc4ccccc4c3)c3cccc4oc5cc6ccccc6cc5c34)cc21 ; CC1(C)c2ccccc2-c2ccc(N(c3ccc(-c4cccc5ccccc45)cc3)c3ccc4oc5cc6ccccc6cc5c4c3)cc21]

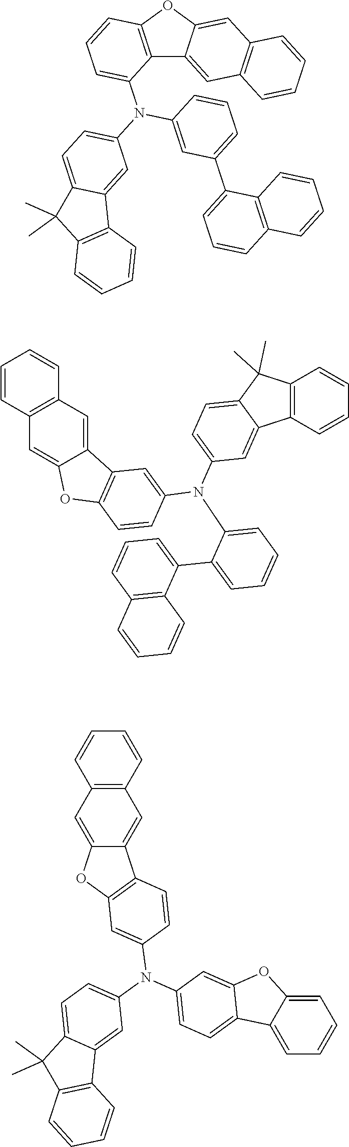 [SMILES: CC1(C)c2ccccc2-c2cc(N(c3cccc(-c4cccc5ccccc45)c3)c3cccc4oc5cc6ccccc6cc5c34)ccc21 ; CC1(C)c2ccccc2-c2cc(N(c3ccc4oc5cc6ccccc6cc5c4c3)c3ccccc3-c3cccc4ccccc34)ccc21 ; CC1(C)c2ccccc2-c2cc(N(c3ccc4c(c3)oc3ccccc34)c3ccc4c(c3)oc3cc5ccccc5cc34)ccc21]

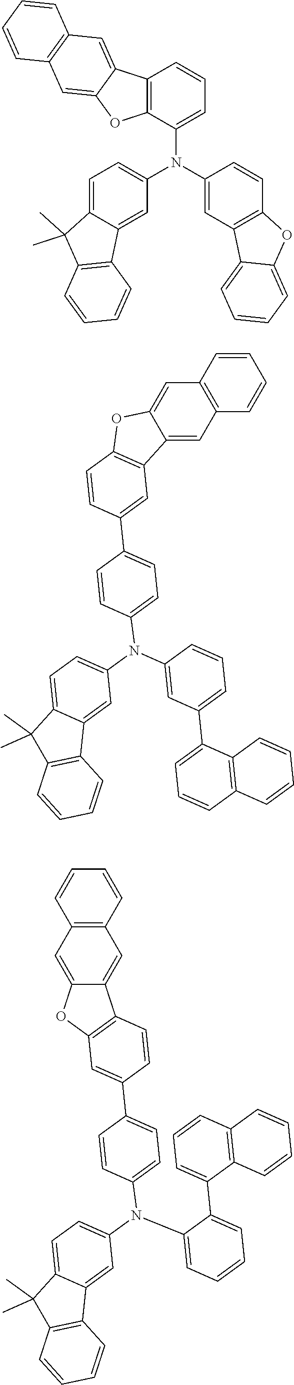

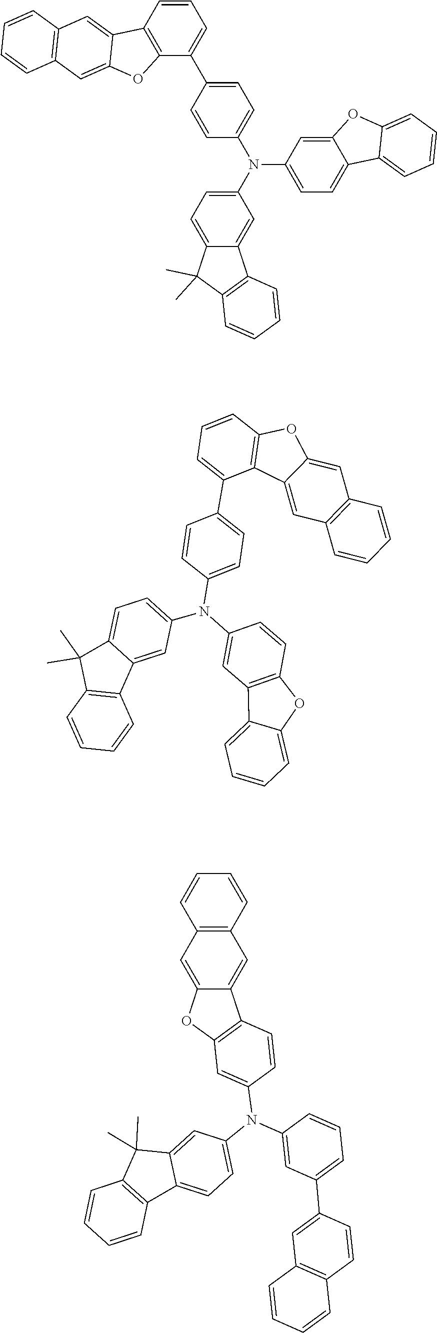 [SMILES: CC1(C)c2ccccc2-c2cc(N(c3ccc(-c4cccc5c4oc4cc6ccccc6cc45)cc3)c3ccc4c(c3)oc3ccccc34)ccc21 ; CC1(C)c2ccccc2-c2cc(N(c3ccc(-c4cccc5oc6cc7ccccc7cc6c45)cc3)c3ccc4oc5ccccc5c4c3)ccc21 ; CC1(C)c2ccccc2-c2ccc(N(c3cccc(-c4ccc5ccccc5c4)c3)c3ccc4c(c3)oc3cc5ccccc5cc34)cc21]

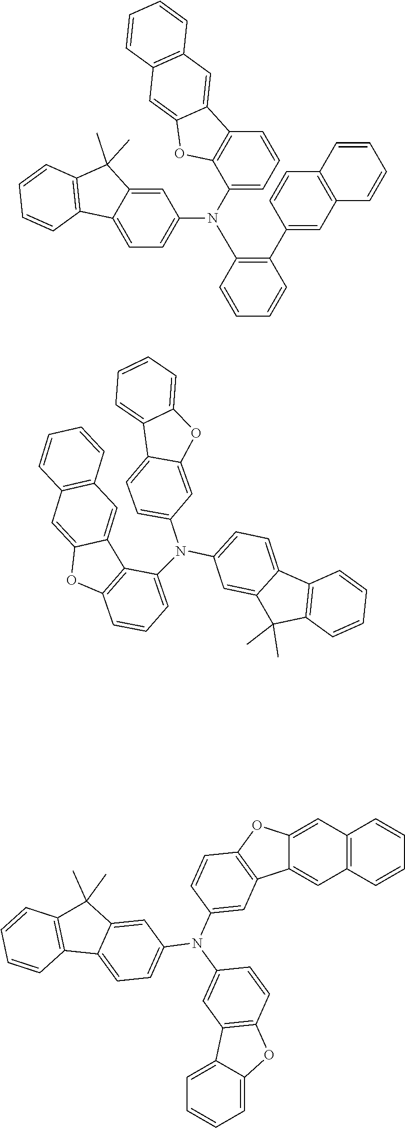 [SMILES: CC1(C)c2ccccc2-c2ccc(N(c3ccccc3-c3ccc4ccccc4c3)c3cccc4c3oc3cc5ccccc5cc34)cc21 ; CC1(C)c2ccccc2-c2ccc(N(c3ccc4c(c3)oc3ccccc34)c3cccc4oc5cc6ccccc6cc5c34)cc21 ; CC1(C)c2ccccc2-c2ccc(N(c3ccc4oc5ccccc5c4c3)c3ccc4oc5cc6ccccc6cc5c4c3)cc21]

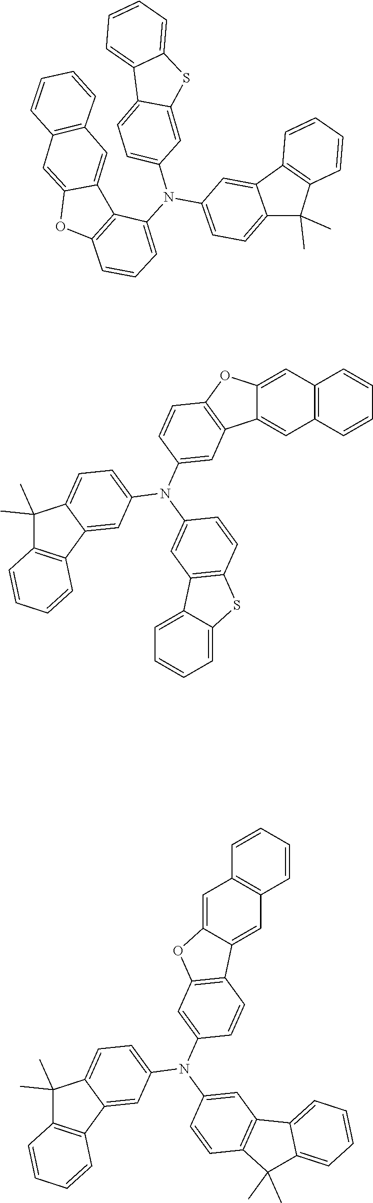 [SMILES: CC1(C)c2ccccc2-c2cc(N(c3ccc4c(c3)sc3ccccc34)c3cccc4oc5cc6ccccc6cc5c34)ccc21 ; CC1(C)c2ccccc2-c2cc(N(c3ccc4oc5cc6ccccc6cc5c4c3)c3ccc4sc5ccccc5c4c3)ccc21 ; CC1(C)c2ccccc2-c2cc(N(c3ccc4c(c3)-c3ccccc3C4(C)C)c3ccc4c(c3)oc3cc5ccccc5cc34)ccc21]

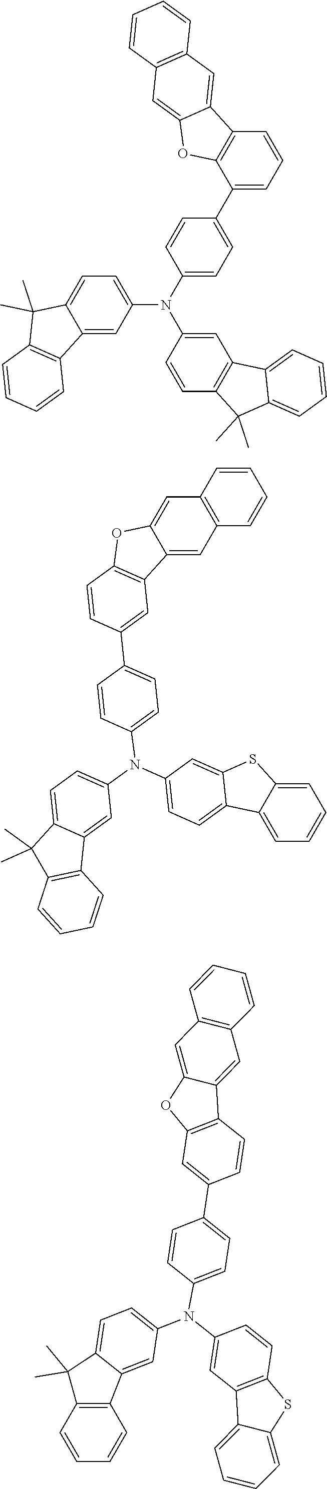

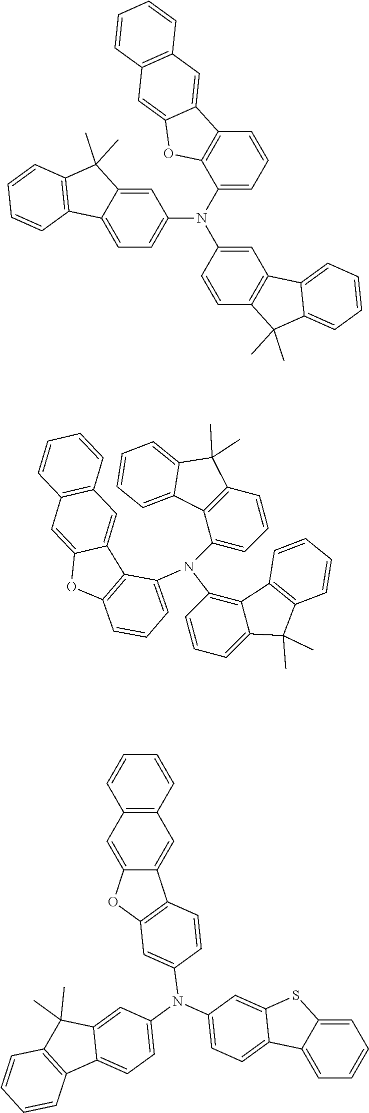 [SMILES: CC1(C)c2ccccc2-c2cc(N(c3ccc4c(c3)C(C)(C)c3ccccc3-4)c3cccc4c3oc3cc5ccccc5cc34)ccc21 ; CC1(C)c2ccccc2-c2c(N(c3cccc4c3-c3ccccc3C4(C)C)c3cccc4oc5cc6ccccc6cc5c34)cccc21 ; CC1(C)c2ccccc2-c2ccc(N(c3ccc4c(c3)oc3cc5ccccc5cc34)c3ccc4c(c3)sc3ccccc34)cc21]

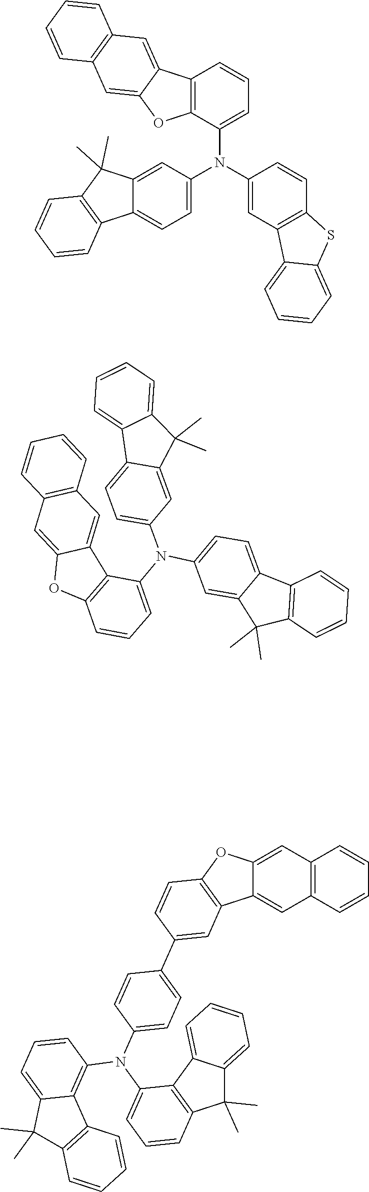 [SMILES: CC1(C)c2ccccc2-c2ccc(N(c3ccc4sc5ccccc5c4c3)c3cccc4c3oc3cc5ccccc5cc34)cc21 ; CC1(C)c2ccccc2-c2ccc(N(c3ccc4c(c3)C(C)(C)c3ccccc3-4)c3cccc4oc5cc6ccccc6cc5c34)cc21 ; CC1(C)c2ccccc2-c2c(N(c3ccc(-c4ccc5oc6cc7ccccc7cc6c5c4)cc3)c3cccc4c3-c3ccccc3C4(C)C)cccc21]

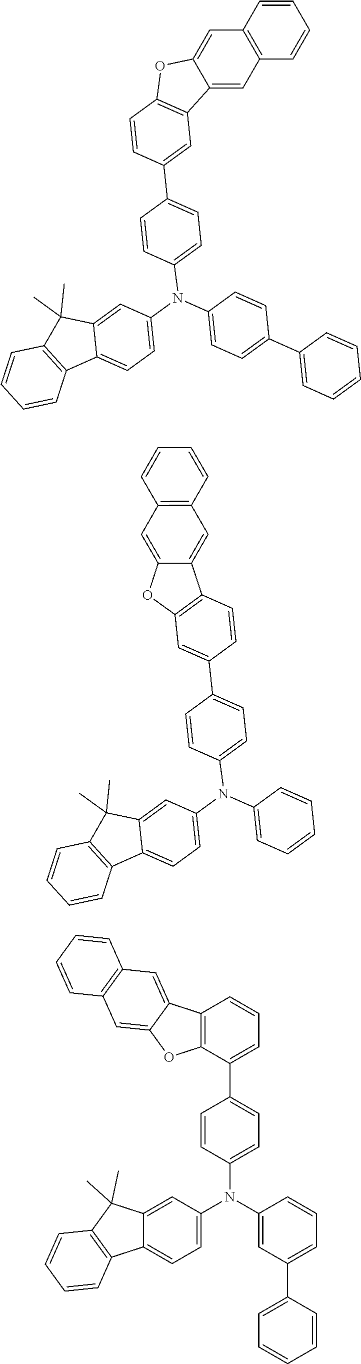

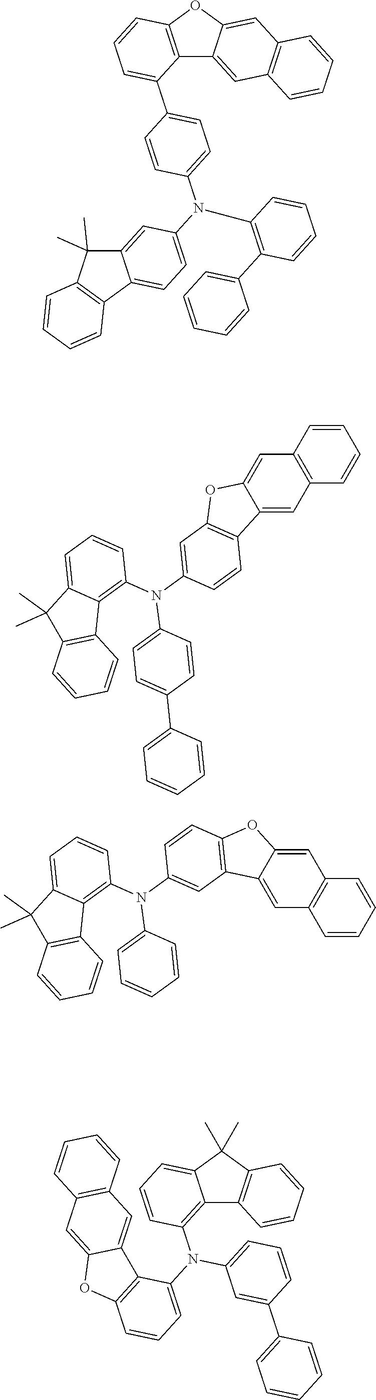

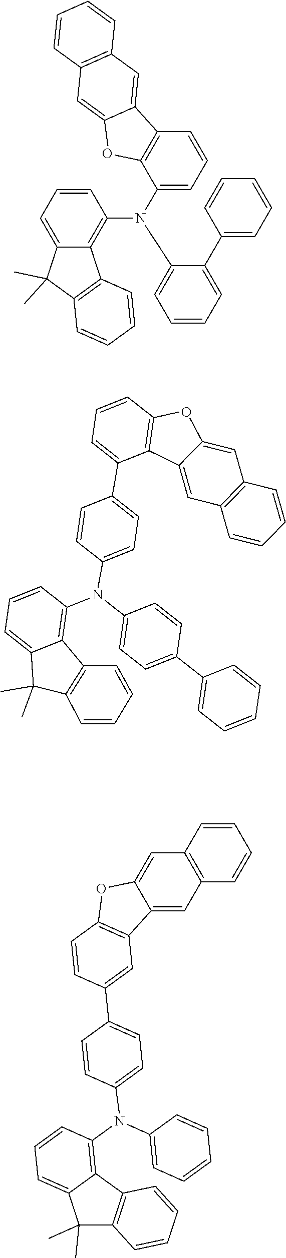 [SMILES: CC1(C)c2ccccc2-c2c(N(c3ccccc3-c3ccccc3)c3cccc4c3oc3cc5ccccc5cc34)cccc21 ; CC1(C)c2ccccc2-c2c(N(c3ccc(-c4ccccc4)cc3)c3ccc(-c4cccc5oc6cc7ccccc7cc6c45)cc3)cccc21 ; CC1(C)c2ccccc2-c2c(N(c3ccccc3)c3ccc(-c4ccc5oc6cc7ccccc7cc6c5c4)cc3)cccc21]

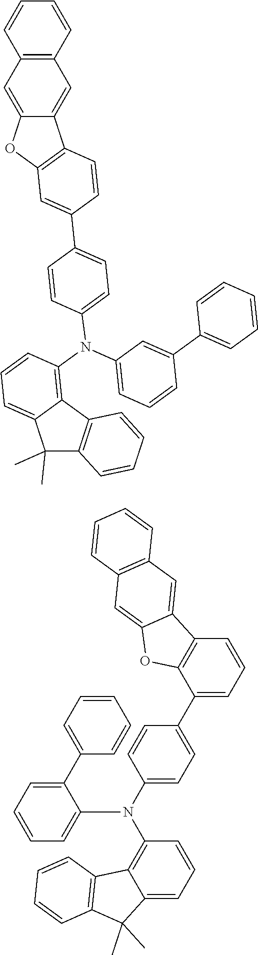

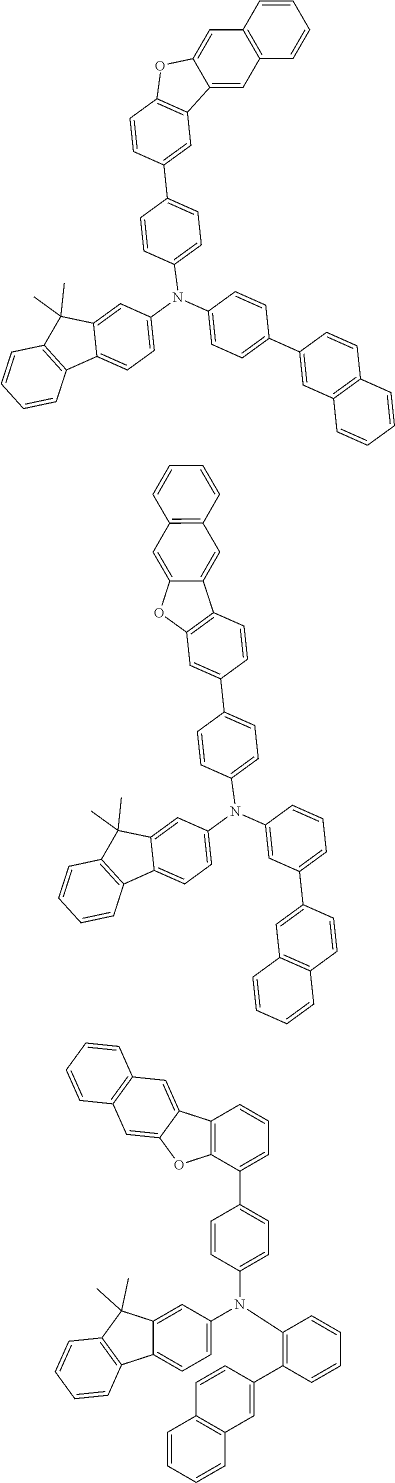 [SMILES: CC1(C)c2ccccc2-c2ccc(N(c3ccc(-c4ccc5ccccc5c4)cc3)c3ccc(-c4ccc5oc6cc7ccccc7cc6c5c4)cc3)cc21 ; CC1(C)c2ccccc2-c2ccc(N(c3ccc(-c4ccc5c(c4)oc4cc6ccccc6cc45)cc3)c3cccc(-c4ccc5ccccc5c4)c3)cc21 ; CC1(C)c2ccccc2-c2ccc(N(c3ccc(-c4cccc5c4oc4cc6ccccc6cc45)cc3)c3ccccc3-c3ccc4ccccc4c3)cc21]

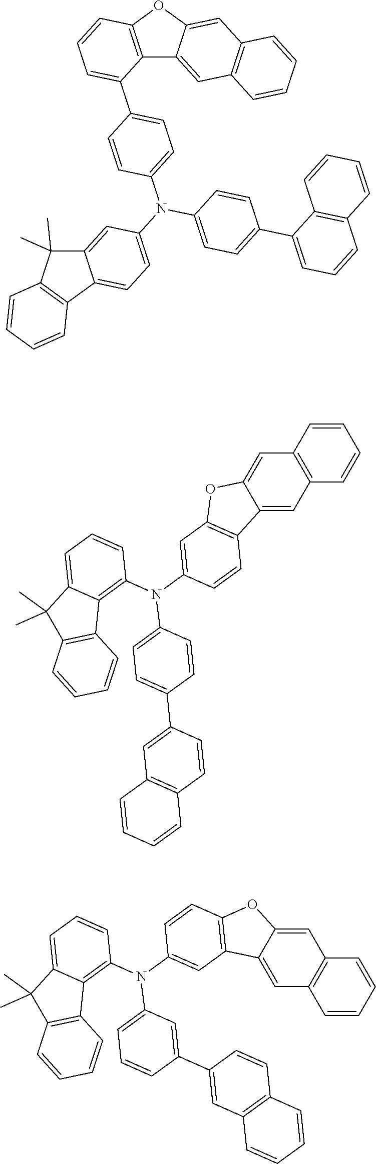 [SMILES: CC1(C)c2ccccc2-c2ccc(N(c3ccc(-c4cccc5ccccc45)cc3)c3ccc(-c4cccc5oc6cc7ccccc7cc6c45)cc3)cc21 ; CC1(C)c2ccccc2-c2c(N(c3ccc(-c4ccc5ccccc5c4)cc3)c3ccc4c(c3)oc3cc5ccccc5cc34)cccc21 ; CC1(C)c2ccccc2-c2c(N(c3cccc(-c4ccc5ccccc5c4)c3)c3ccc4oc5cc6ccccc6cc5c4c3)cccc21]

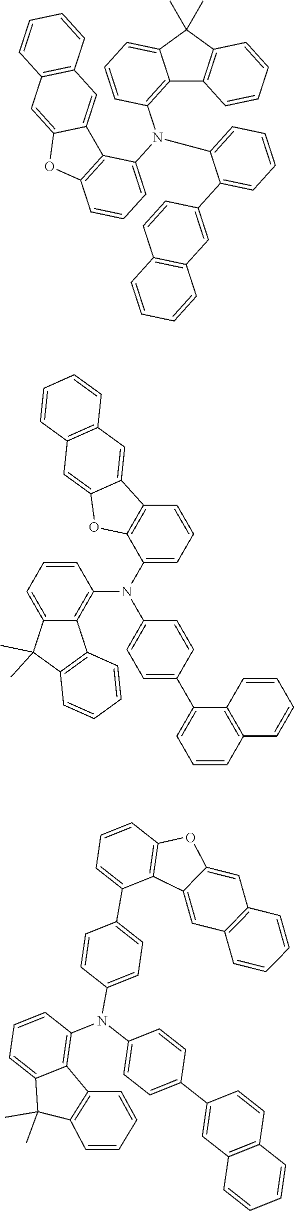 [SMILES: CC1(C)c2ccccc2-c2c(N(c3ccccc3-c3ccc4ccccc4c3)c3cccc4oc5cc6ccccc6cc5c34)cccc21 ; CC1(C)c2ccccc2-c2c(N(c3ccc(-c4cccc5ccccc45)cc3)c3cccc4c3oc3cc5ccccc5cc34)cccc21 ; CC1(C)c2ccccc2-c2c(N(c3ccc(-c4ccc5ccccc5c4)cc3)c3ccc(-c4cccc5oc6cc7ccccc7cc6c45)cc3)cccc21]

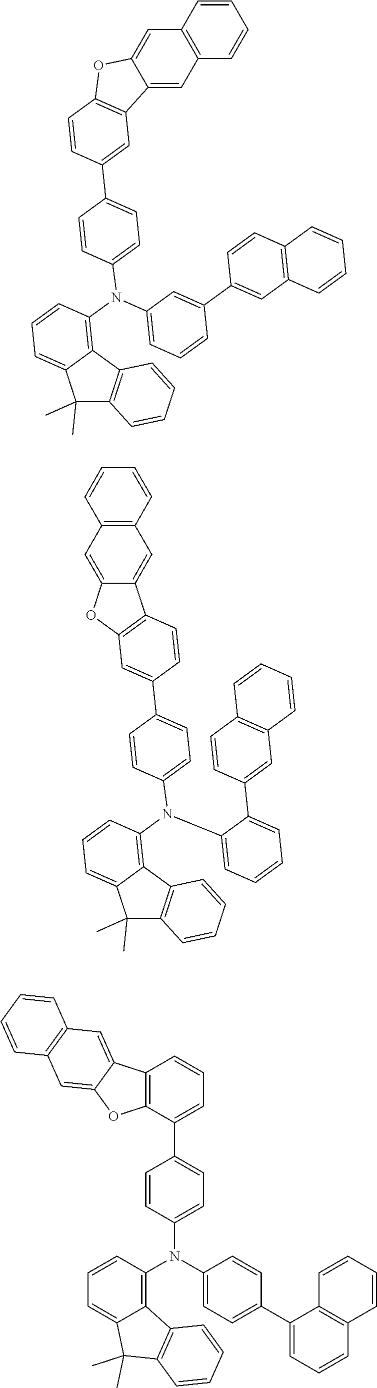

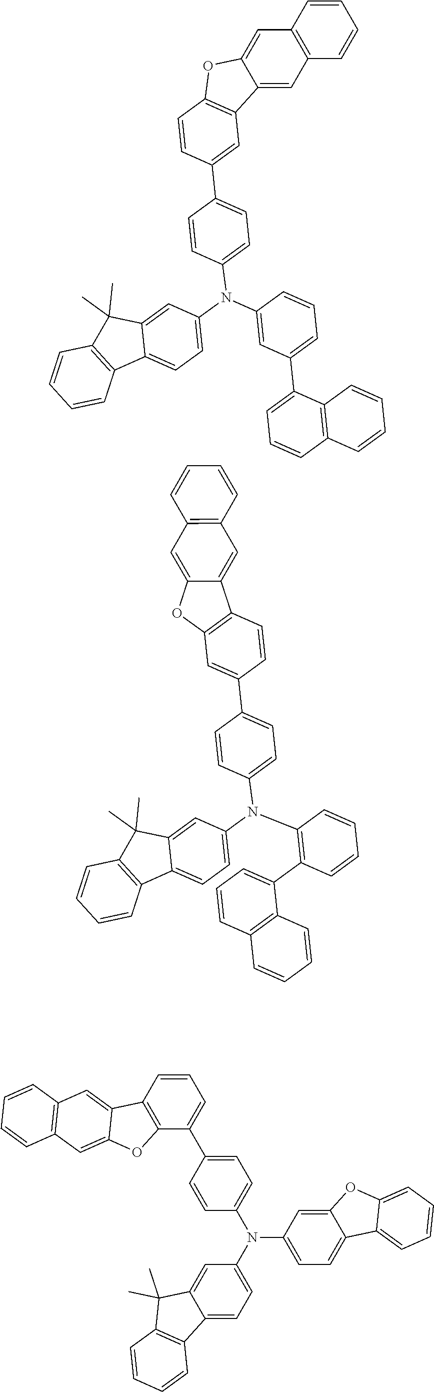 [SMILES: CC1(C)c2ccccc2-c2ccc(N(c3ccc(-c4ccc5oc6cc7ccccc7cc6c5c4)cc3)c3cccc(-c4cccc5ccccc45)c3)cc21 ; CC1(C)c2ccccc2-c2ccc(N(c3ccc(-c4ccc5c(c4)oc4cc6ccccc6cc45)cc3)c3ccccc3-c3cccc4ccccc34)cc21 ; CC1(C)c2ccccc2-c2ccc(N(c3ccc(-c4cccc5c4oc4cc6ccccc6cc45)cc3)c3ccc4c(c3)oc3ccccc34)cc21]

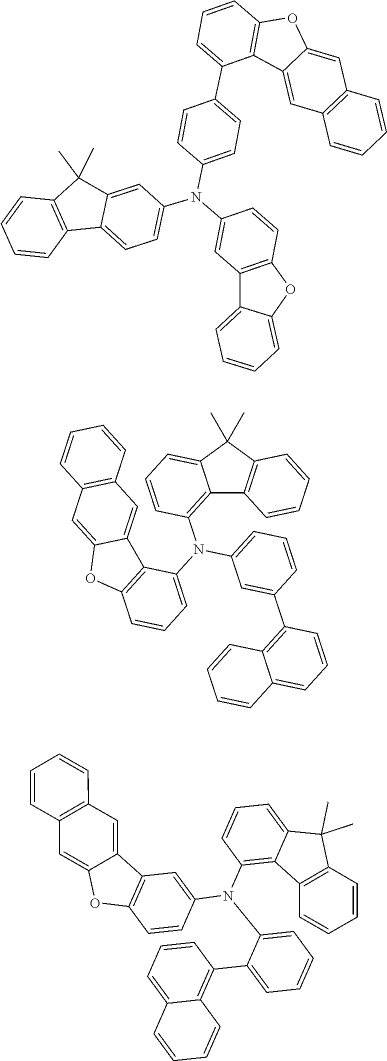

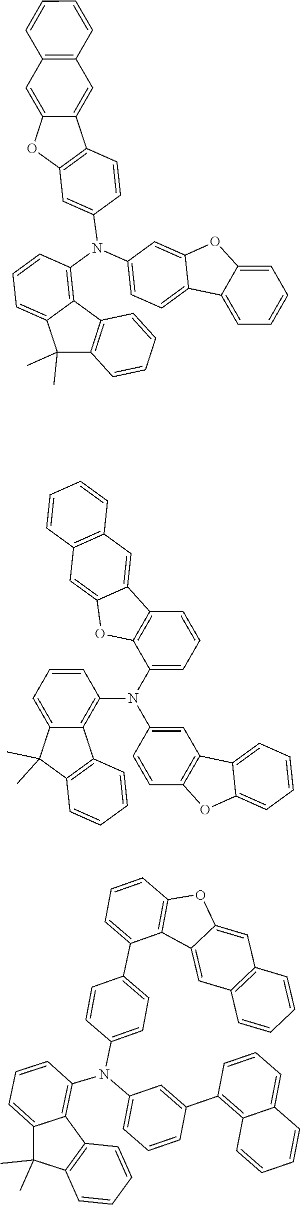 [SMILES: CC1(C)c2ccccc2-c2c(N(c3ccc4c(c3)oc3ccccc34)c3ccc4c(c3)oc3cc5ccccc5cc34)cccc21 ; CC1(C)c2ccccc2-c2c(N(c3ccc4oc5ccccc5c4c3)c3cccc4c3oc3cc5ccccc5cc34)cccc21 ; CC1(C)c2ccccc2-c2c(N(c3ccc(-c4cccc5oc6cc7ccccc7cc6c45)cc3)c3cccc(-c4cccc5ccccc45)c3)cccc21]

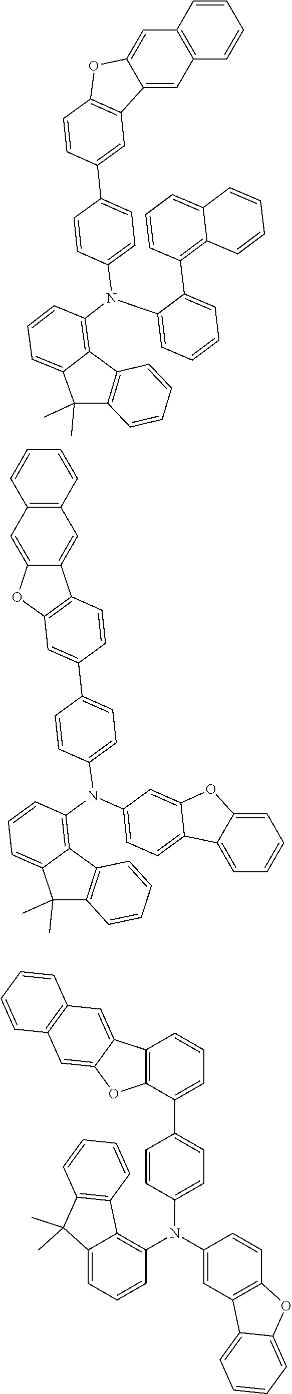

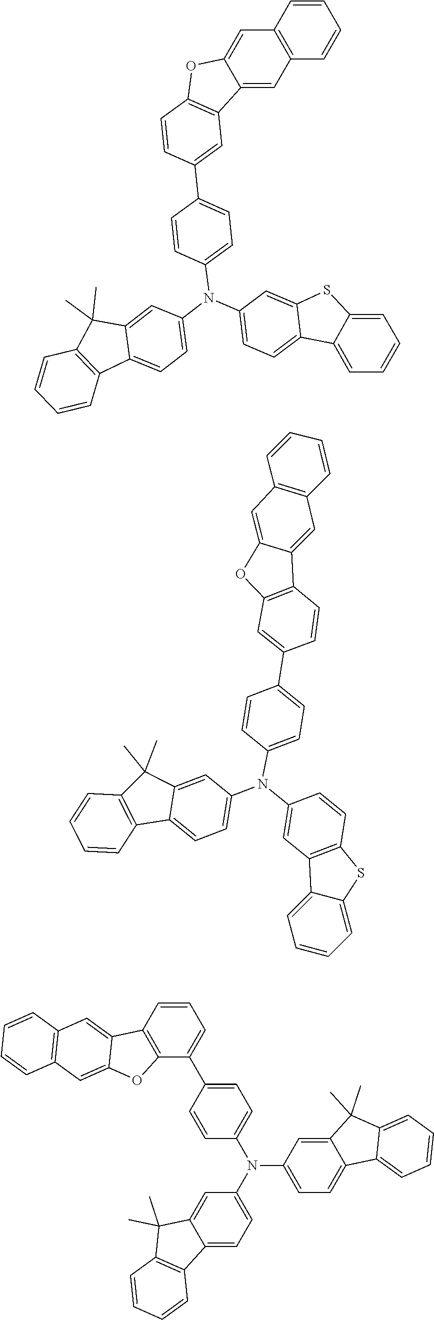 [SMILES: CC1(C)c2ccccc2-c2ccc(N(c3ccc(-c4ccc5oc6cc7ccccc7cc6c5c4)cc3)c3ccc4c(c3)sc3ccccc34)cc21 ; CC1(C)c2ccccc2-c2ccc(N(c3ccc(-c4ccc5c(c4)oc4cc6ccccc6cc45)cc3)c3ccc4sc5ccccc5c4c3)cc21 ; CC1(C)c2ccccc2-c2ccc(N(c3ccc(-c4cccc5c4oc4cc6ccccc6cc45)cc3)c3ccc4c(c3)C(C)(C)c3ccccc3-4)cc21]

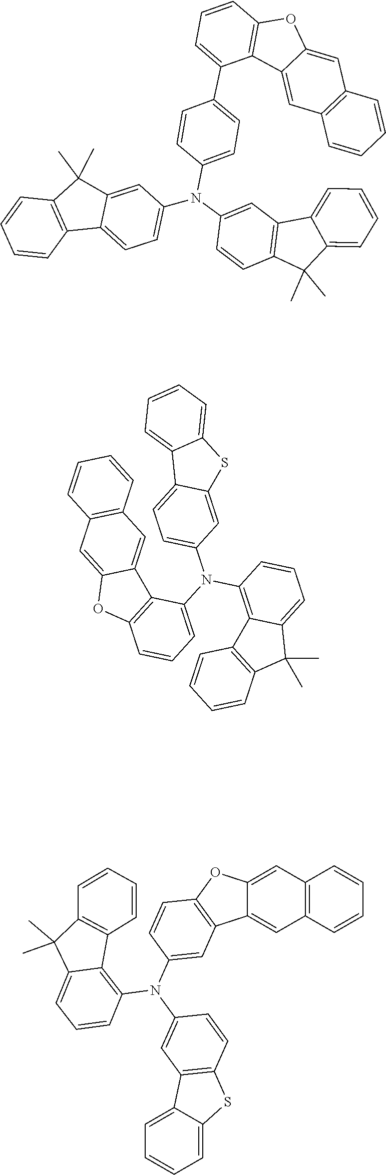

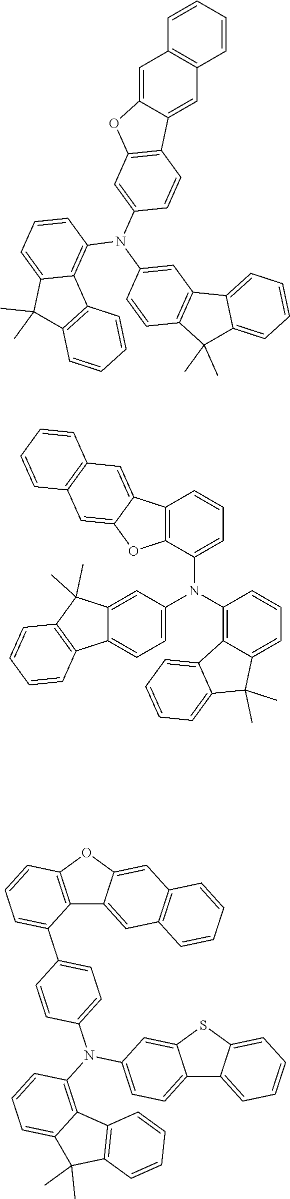 [SMILES: CC1(C)c2ccccc2-c2cc(N(c3ccc4c(c3)oc3cc5ccccc5cc34)c3cccc4c3-c3ccccc3C4(C)C)ccc21 ; CC1(C)c2ccccc2-c2ccc(N(c3cccc4c3-c3ccccc3C4(C)C)c3cccc4c3oc3cc5ccccc5cc34)cc21 ; CC1(C)c2ccccc2-c2c(N(c3ccc(-c4cccc5oc6cc7ccccc7cc6c45)cc3)c3ccc4c(c3)sc3ccccc34)cccc21]

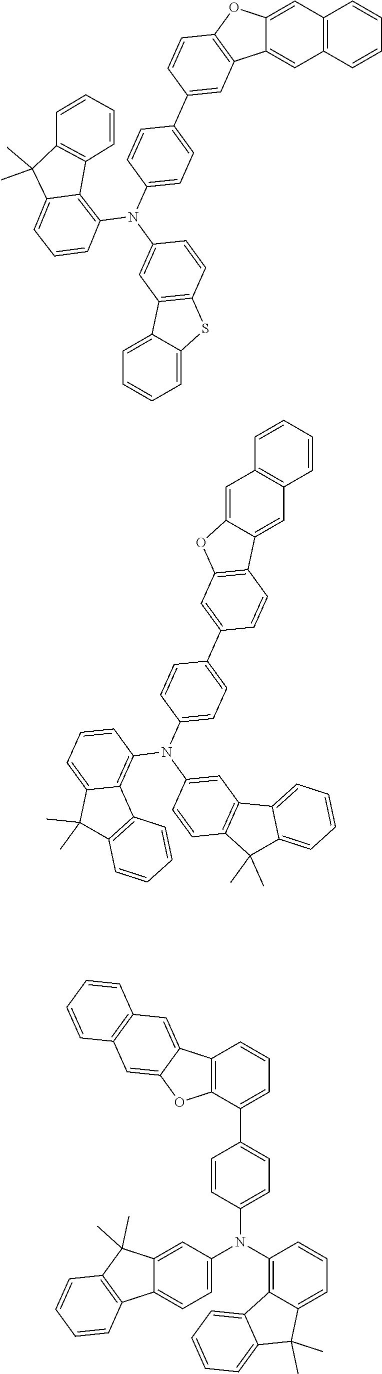 [SMILES: CC1(C)c2ccccc2-c2c(N(c3ccc(-c4ccc5oc6cc7ccccc7cc6c5c4)cc3)c3ccc4sc5ccccc5c4c3)cccc21 ; CC1(C)c2ccccc2-c2cc(N(c3ccc(-c4ccc5c(c4)oc4cc6ccccc6cc45)cc3)c3cccc4c3-c3ccccc3C4(C)C)ccc21 ; CC1(C)c2ccccc2-c2ccc(N(c3ccc(-c4cccc5c4oc4cc6ccccc6cc45)cc3)c3cccc4c3-c3ccccc3C4(C)C)cc21]

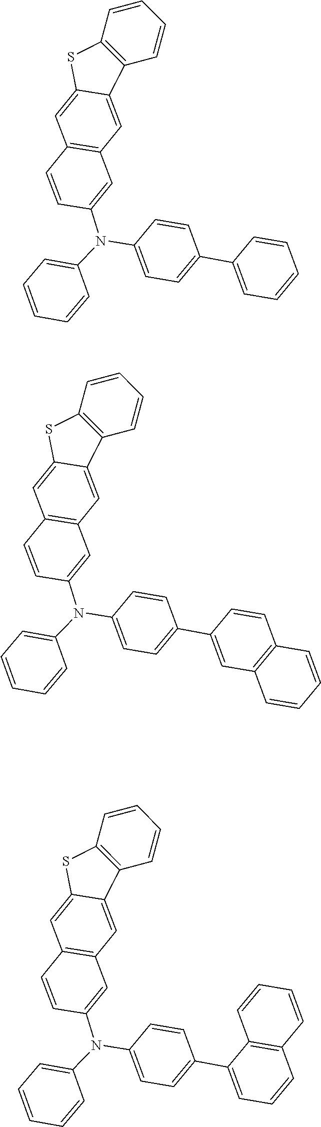 [SMILES: c1ccc(-c2ccc(N(c3ccccc3)c3ccc4cc5sc6ccccc6c5cc4c3)cc2)cc1 ; c1ccc(N(c2ccc(-c3ccc4ccccc4c3)cc2)c2ccc3cc4sc5ccccc5c4cc3c2)cc1 ; c1ccc(N(c2ccc(-c3cccc4ccccc34)cc2)c2ccc3cc4sc5ccccc5c4cc3c2)cc1]

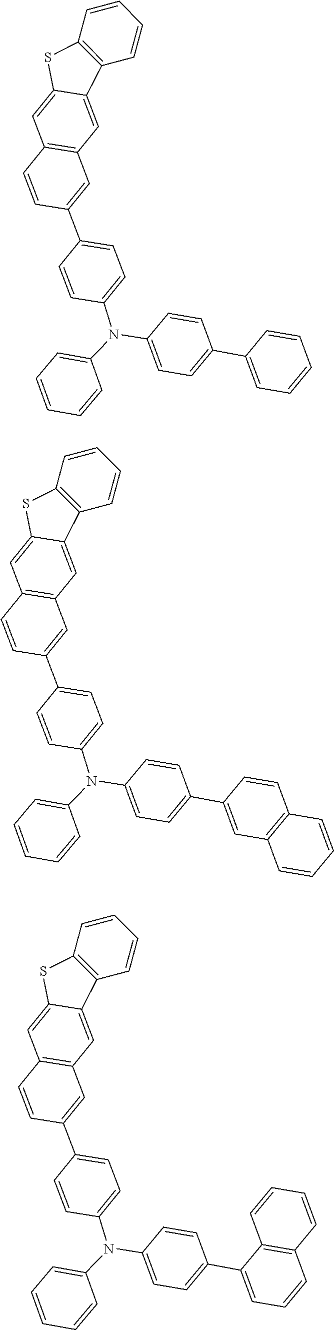

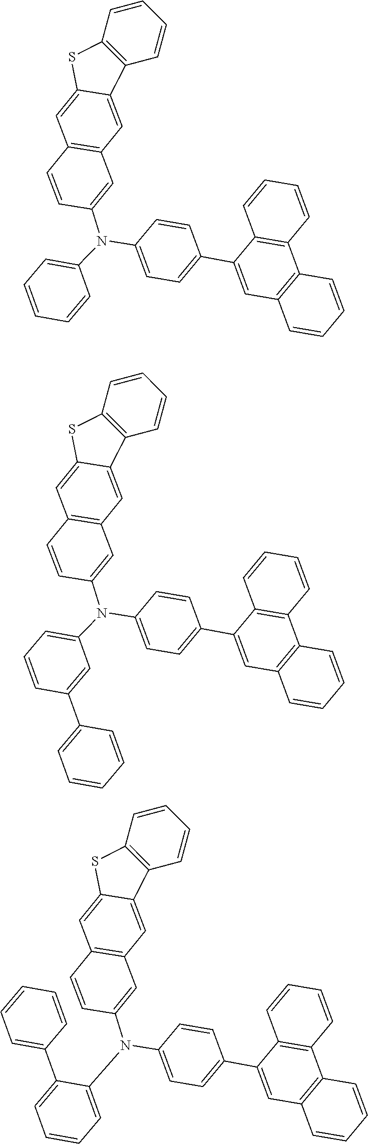

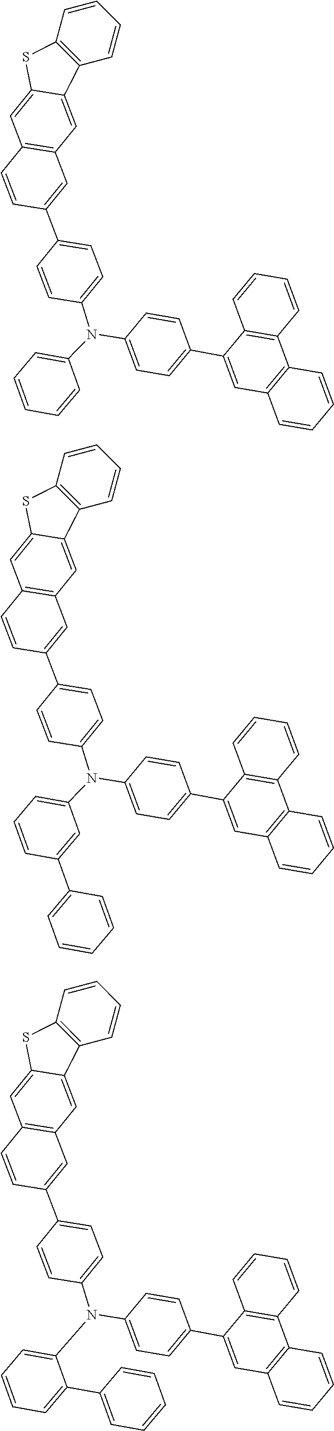

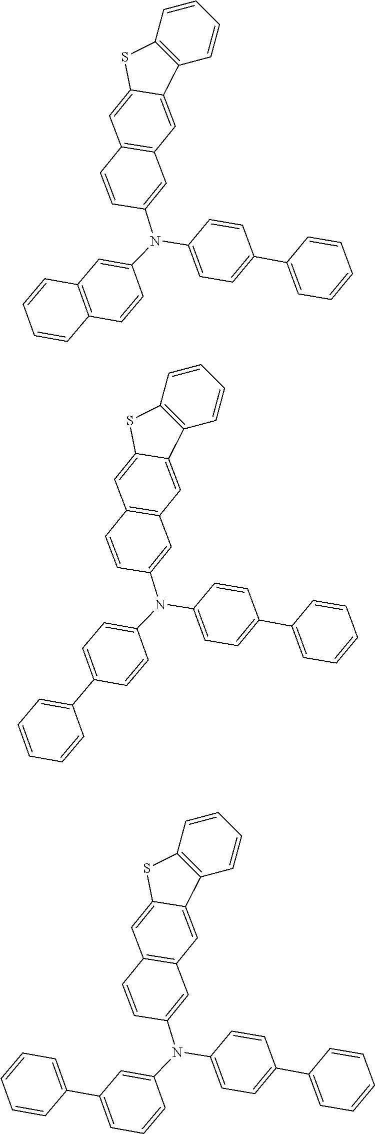 [SMILES: c1ccc(-c2ccc(N(c3ccc4ccccc4c3)c3ccc4cc5sc6ccccc6c5cc4c3)cc2)cc1 ; c1ccc(-c2ccc(N(c3ccc(-c4ccccc4)cc3)c3ccc4cc5sc6ccccc6c5cc4c3)cc2)cc1 ; c1ccc(-c2ccc(N(c3cccc(-c4ccccc4)c3)c3ccc4cc5sc6ccccc6c5cc4c3)cc2)cc1]

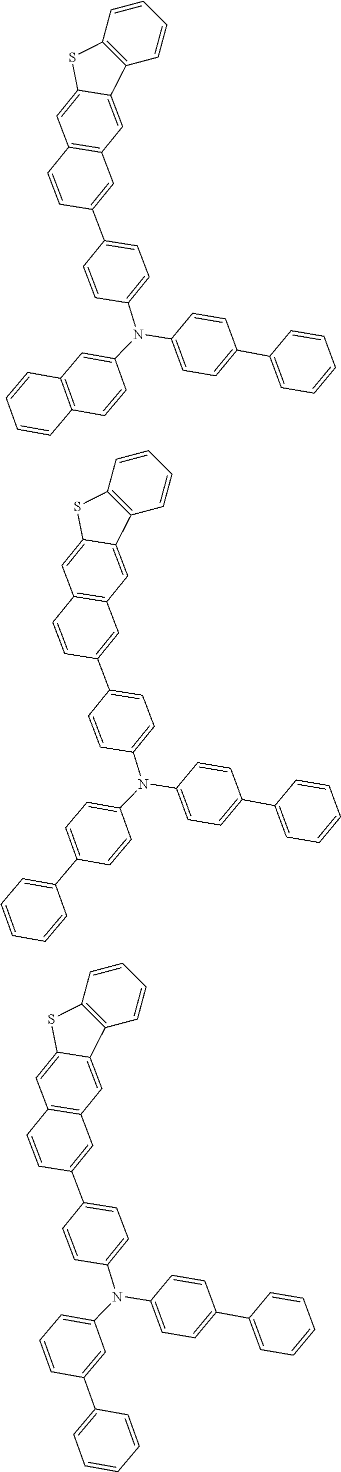

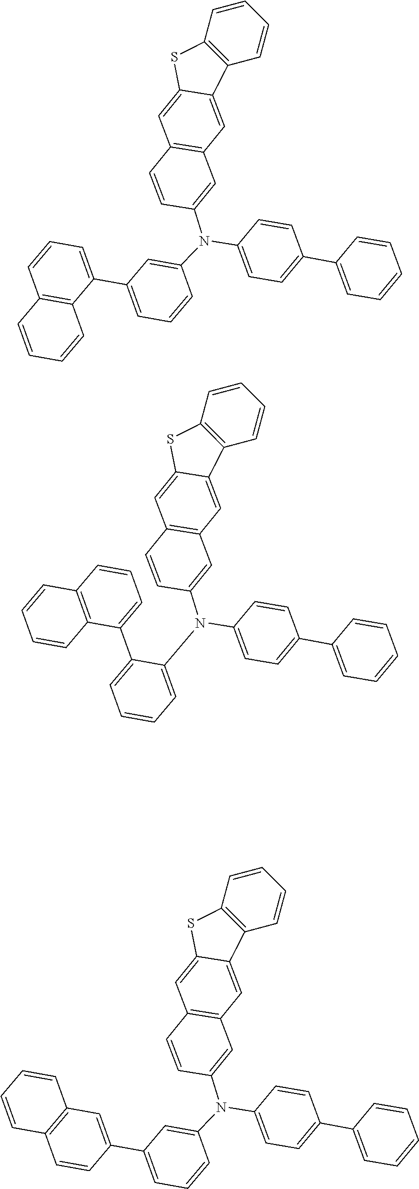 [SMILES: c1ccc(-c2ccc(N(c3cccc(-c4cccc5ccccc45)c3)c3ccc4cc5sc6ccccc6c5cc4c3)cc2)cc1 ; c1ccc(-c2ccc(N(c3ccc4cc5sc6ccccc6c5cc4c3)c3ccccc3-c3cccc4ccccc34)cc2)cc1 ; c1ccc(-c2ccc(N(c3cccc(-c4ccc5ccccc5c4)c3)c3ccc4cc5sc6ccccc6c5cc4c3)cc2)cc1]

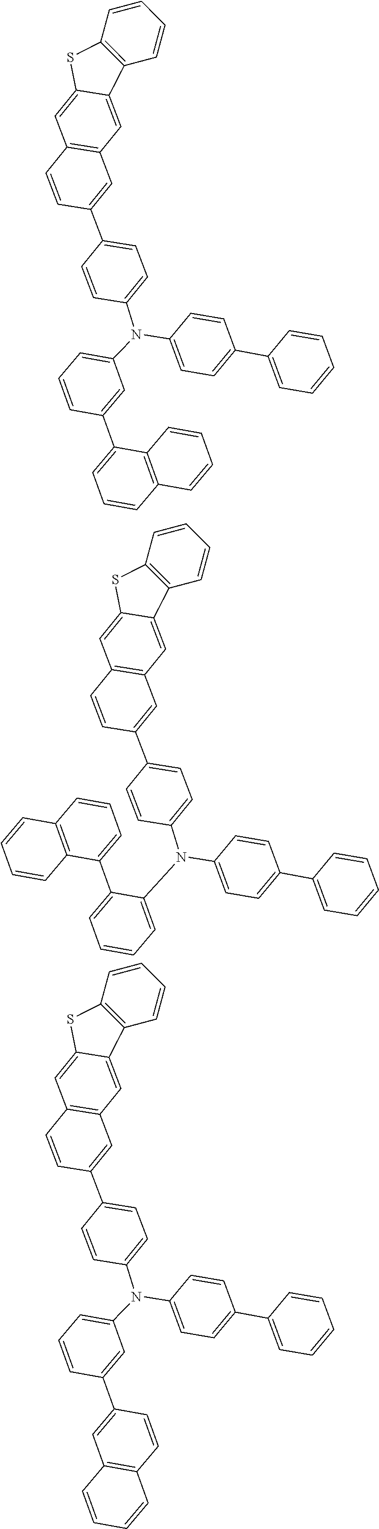

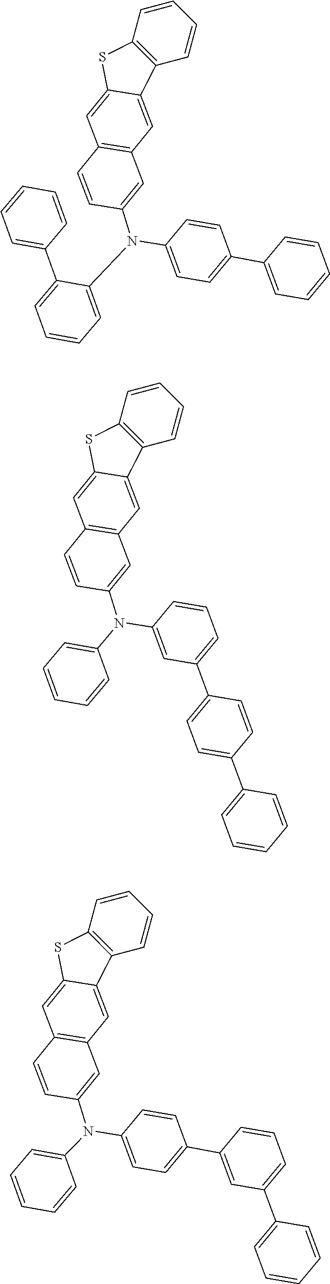 [SMILES: c1ccc(-c2ccc(N(c3ccc4cc5sc6ccccc6c5cc4c3)c3ccccc3-c3ccccc3)cc2)cc1 ; c1ccc(-c2ccc(-c3cccc(N(c4ccccc4)c4ccc5cc6sc7ccccc7c6cc5c4)c3)cc2)cc1 ; c1ccc(-c2cccc(-c3ccc(N(c4ccccc4)c4ccc5cc6sc7ccccc7c6cc5c4)cc3)c2)cc1]

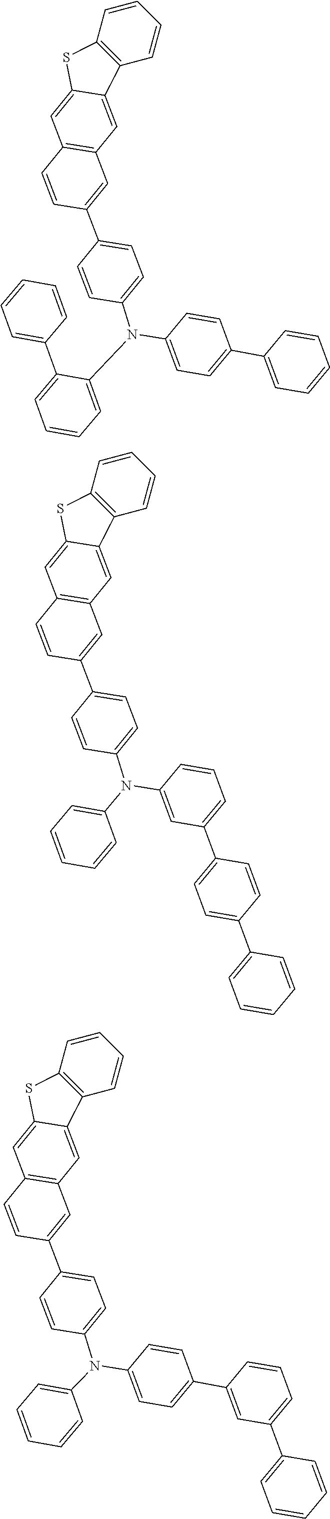

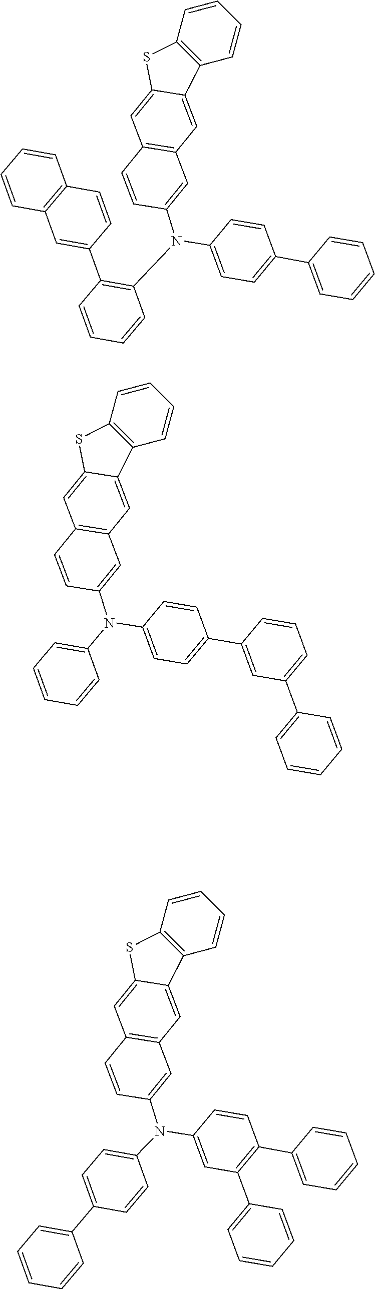 [SMILES: c1ccc(-c2ccc(N(c3ccc4cc5sc6ccccc6c5cc4c3)c3ccccc3-c3ccc4ccccc4c3)cc2)cc1 ; c1ccc(-c2cccc(-c3ccc(N(c4ccccc4)c4ccc5cc6sc7ccccc7c6cc5c4)cc3)c2)cc1 ; c1ccc(-c2ccc(N(c3ccc(-c4ccccc4)c(-c4ccccc4)c3)c3ccc4cc5sc6ccccc6c5cc4c3)cc2)cc1]

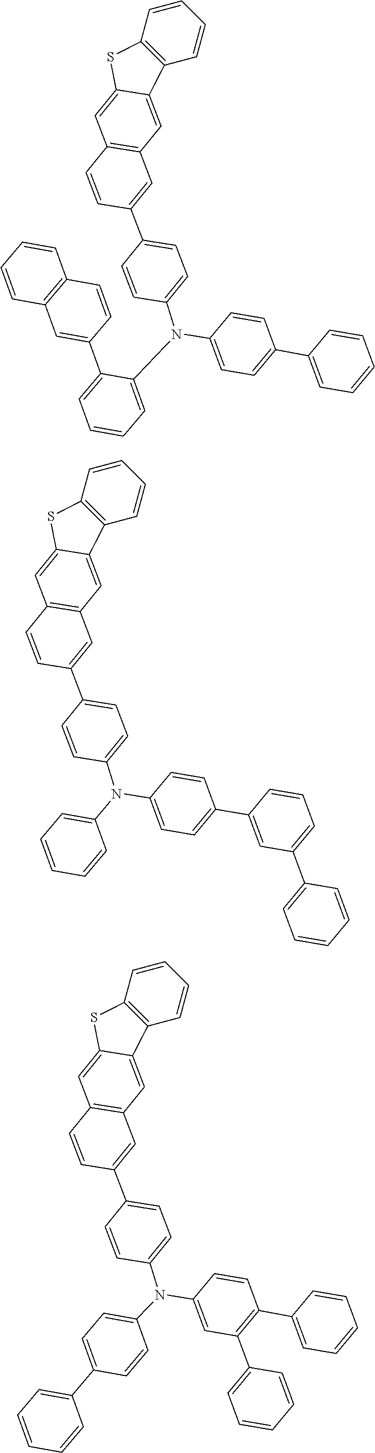

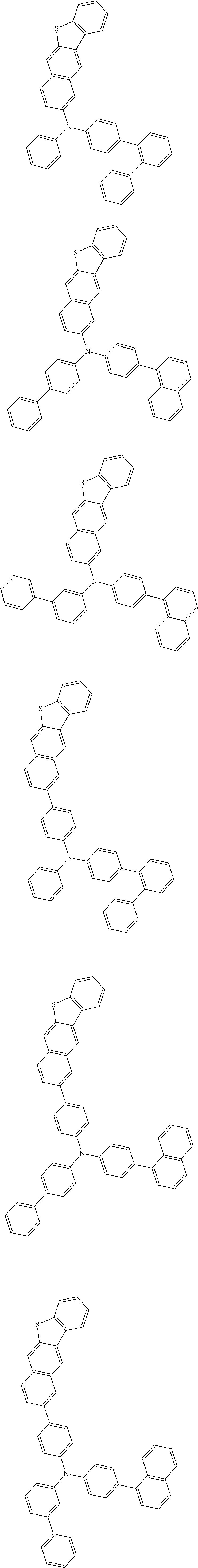

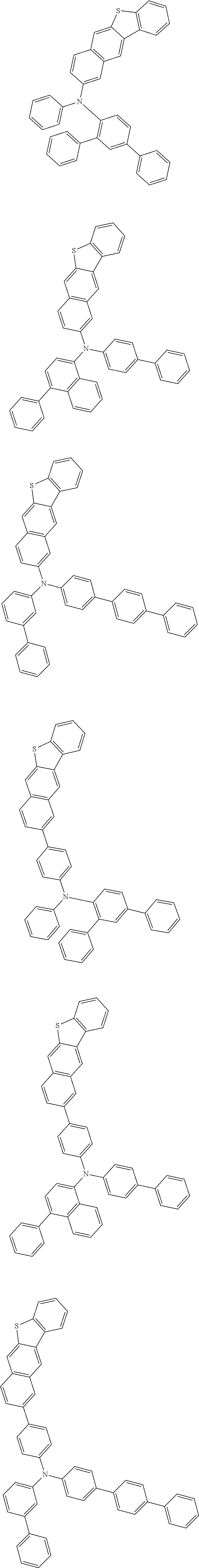 [SMILES: c1ccc(-c2ccc(N(c3ccccc3)c3ccc4cc5sc6ccccc6c5cc4c3)c(-c3ccccc3)c2)cc1 ; c1ccc(-c2ccc(N(c3ccc4cc5sc6ccccc6c5cc4c3)c3ccc(-c4ccccc4)c4ccccc34)cc2)cc1 ; c1ccc(-c2ccc(-c3ccc(N(c4cccc(-c5ccccc5)c4)c4ccc5cc6sc7ccccc7c6cc5c4)cc3)cc2)cc1 ; c1ccc(-c2ccc(N(c3ccccc3)c3ccc(-c4ccc5cc6sc7ccccc7c6cc5c4)cc3)c(-c3ccccc3)c2)cc1 ; c1ccc(-c2ccc(N(c3ccc(-c4ccc5cc6sc7ccccc7c6cc5c4)cc3)c3ccc(-c4ccccc4)c4ccccc34)cc2)cc1 ; c1ccc(-c2ccc(-c3ccc(N(c4ccc(-c5ccc6cc7sc8ccccc8c7cc6c5)cc4)c4cccc(-c5ccccc5)c4)cc3)cc2)cc1]

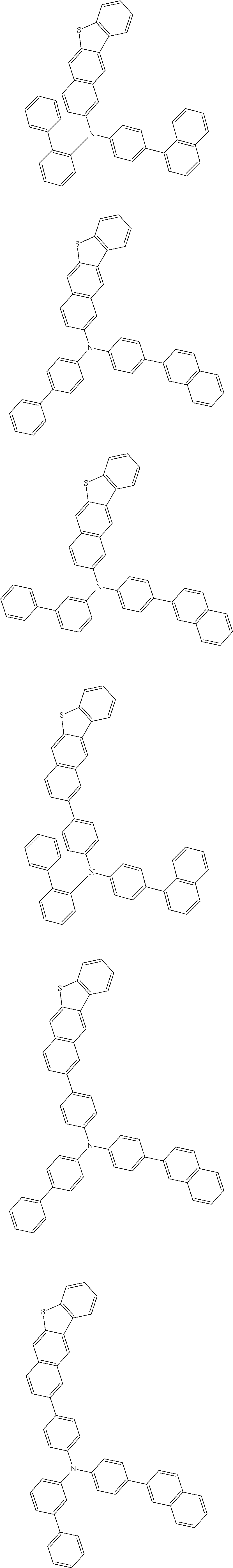 [SMILES: c1ccc(-c2ccccc2N(c2ccc(-c3cccc4ccccc34)cc2)c2ccc3cc4sc5ccccc5c4cc3c2)cc1 ; c1ccc(-c2ccc(N(c3ccc(-c4ccc5ccccc5c4)cc3)c3ccc4cc5sc6ccccc6c5cc4c3)cc2)cc1 ; c1ccc(-c2cccc(N(c3ccc(-c4ccc5ccccc5c4)cc3)c3ccc4cc5sc6ccccc6c5cc4c3)c2)cc1 ; c1ccc(-c2ccccc2N(c2ccc(-c3ccc4cc5sc6ccccc6c5cc4c3)cc2)c2ccc(-c3cccc4ccccc34)cc2)cc1 ; c1ccc(-c2ccc(N(c3ccc(-c4ccc5ccccc5c4)cc3)c3ccc(-c4ccc5cc6sc7ccccc7c6cc5c4)cc3)cc2)cc1 ; c1ccc(-c2cccc(N(c3ccc(-c4ccc5ccccc5c4)cc3)c3ccc(-c4ccc5cc6sc7ccccc7c6cc5c4)cc3)c2)cc1]

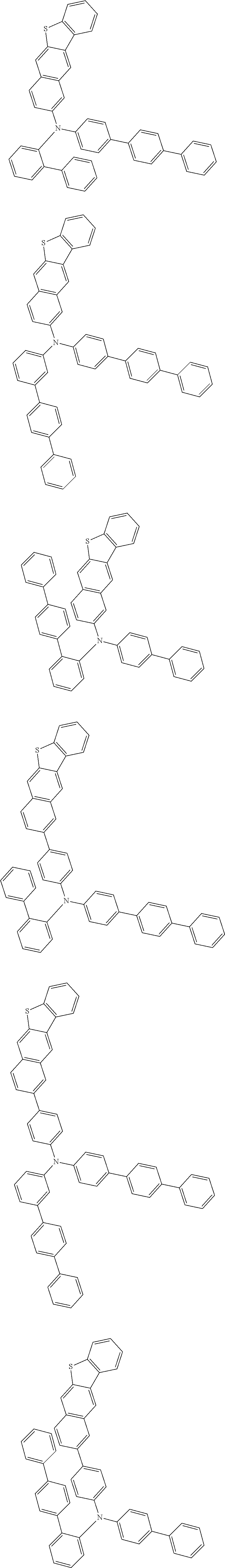

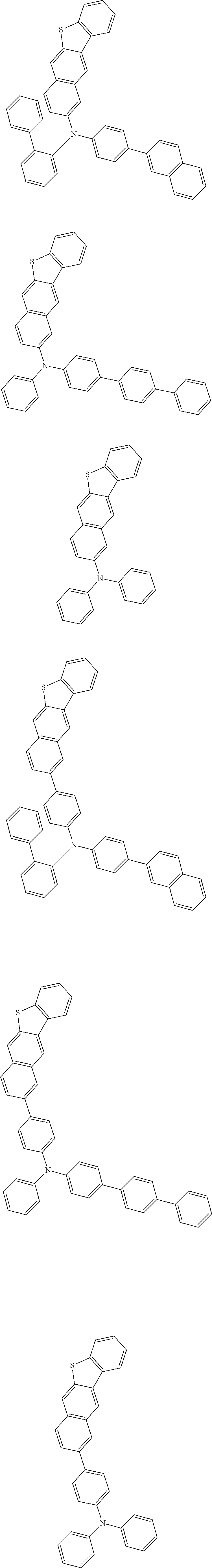 [SMILES: c1ccc(-c2ccccc2N(c2ccc(-c3ccc4ccccc4c3)cc2)c2ccc3cc4sc5ccccc5c4cc3c2)cc1 ; c1ccc(-c2ccc(-c3ccc(N(c4ccccc4)c4ccc5cc6sc7ccccc7c6cc5c4)cc3)cc2)cc1 ; c1ccc(N(c2ccccc2)c2ccc3cc4sc5ccccc5c4cc3c2)cc1 ; c1ccc(-c2ccccc2N(c2ccc(-c3ccc4ccccc4c3)cc2)c2ccc(-c3ccc4cc5sc6ccccc6c5cc4c3)cc2)cc1 ; c1ccc(-c2ccc(-c3ccc(N(c4ccccc4)c4ccc(-c5ccc6cc7sc8ccccc8c7cc6c5)cc4)cc3)cc2)cc1 ; c1ccc(N(c2ccccc2)c2ccc(-c3ccc4cc5sc6ccccc6c5cc4c3)cc2)cc1]

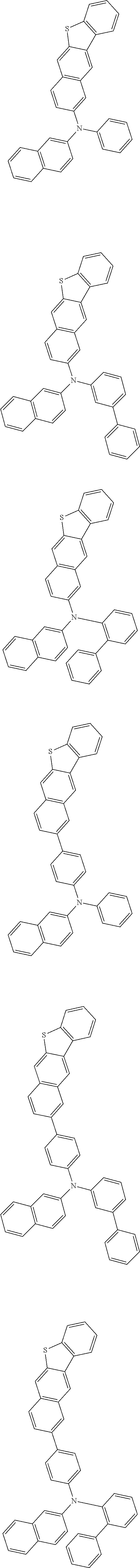 [SMILES: c1ccc(N(c2ccc3ccccc3c2)c2ccc3cc4sc5ccccc5c4cc3c2)cc1 ; c1ccc(-c2cccc(N(c3ccc4ccccc4c3)c3ccc4cc5sc6ccccc6c5cc4c3)c2)cc1 ; c1ccc(-c2ccccc2N(c2ccc3ccccc3c2)c2ccc3cc4sc5ccccc5c4cc3c2)cc1 ; c1ccc(N(c2ccc(-c3ccc4cc5sc6ccccc6c5cc4c3)cc2)c2ccc3ccccc3c2)cc1 ; c1ccc(-c2cccc(N(c3ccc(-c4ccc5cc6sc7ccccc7c6cc5c4)cc3)c3ccc4ccccc4c3)c2)cc1 ; c1ccc(-c2ccccc2N(c2ccc(-c3ccc4cc5sc6ccccc6c5cc4c3)cc2)c2ccc3ccccc3c2)cc1]

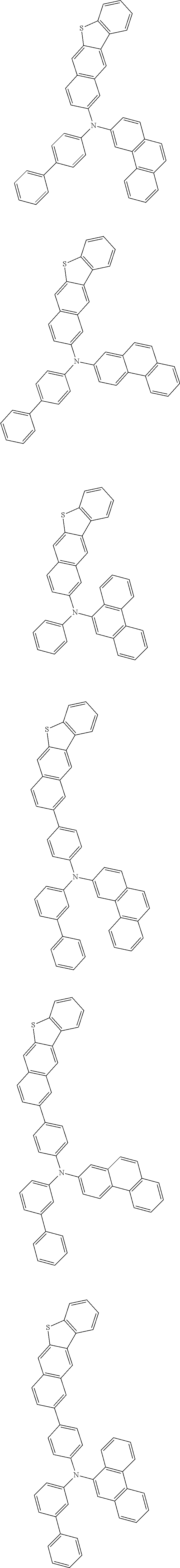 [SMILES: c1ccc(-c2ccc(N(c3ccc4cc5sc6ccccc6c5cc4c3)c3ccc4ccc5ccccc5c4c3)cc2)cc1 ; c1ccc(-c2ccc(N(c3ccc4cc5sc6ccccc6c5cc4c3)c3ccc4c(ccc5ccccc54)c3)cc2)cc1 ; c1ccc(N(c2ccc3cc4sc5ccccc5c4cc3c2)c2cc3ccccc3c3ccccc23)cc1 ; c1ccc(-c2cccc(N(c3ccc(-c4ccc5cc6sc7ccccc7c6cc5c4)cc3)c3ccc4ccc5ccccc5c4c3)c2)cc1 ; c1ccc(-c2cccc(N(c3ccc(-c4ccc5cc6sc7ccccc7c6cc5c4)cc3)c3ccc4c(ccc5ccccc54)c3)c2)cc1 ; c1ccc(-c2cccc(N(c3ccc(-c4ccc5cc6sc7ccccc7c6cc5c4)cc3)c3cc4ccccc4c4ccccc34)c2)cc1]

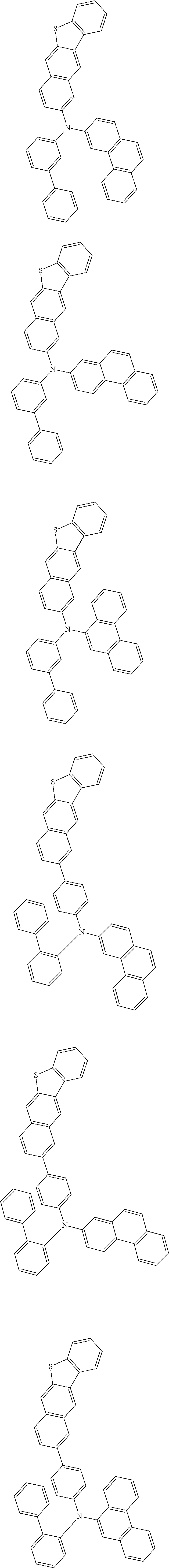

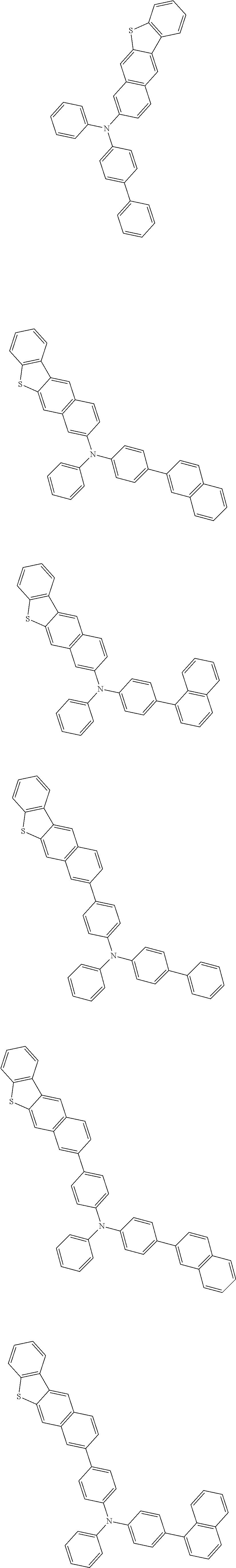 [SMILES: c1ccc(-c2ccc(N(c3ccccc3)c3ccc4cc5c(cc4c3)sc3ccccc35)cc2)cc1 ; c1ccc(N(c2ccc(-c3ccc4ccccc4c3)cc2)c2ccc3cc4c(cc3c2)sc2ccccc24)cc1 ; c1ccc(N(c2ccc(-c3cccc4ccccc34)cc2)c2ccc3cc4c(cc3c2)sc2ccccc24)cc1 ; c1ccc(-c2ccc(N(c3ccccc3)c3ccc(-c4ccc5cc6c(cc5c4)sc4ccccc46)cc3)cc2)cc1 ; c1ccc(N(c2ccc(-c3ccc4ccccc4c3)cc2)c2ccc(-c3ccc4cc5c(cc4c3)sc3ccccc35)cc2)cc1 ; c1ccc(N(c2ccc(-c3ccc4cc5c(cc4c3)sc3ccccc35)cc2)c2ccc(-c3cccc4ccccc34)cc2)cc1]

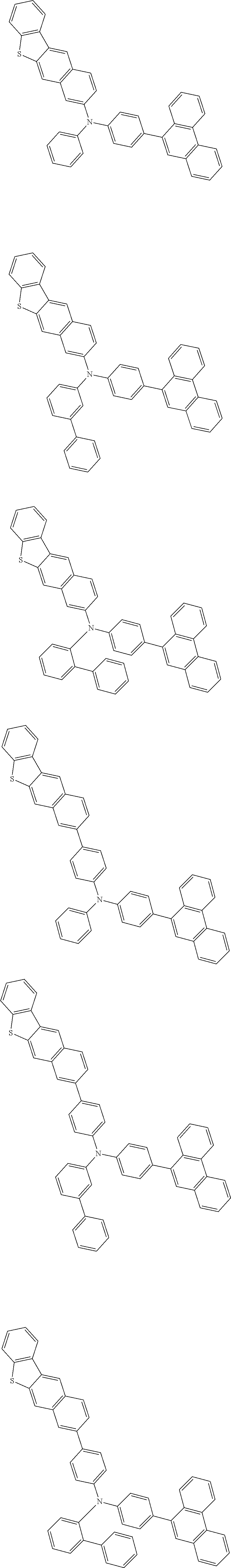

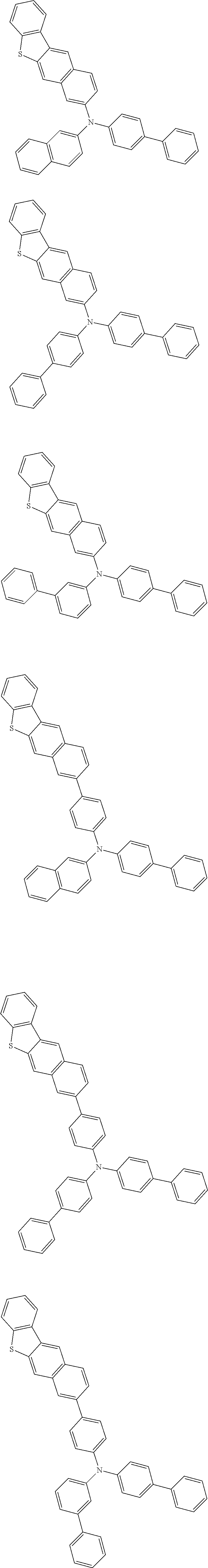

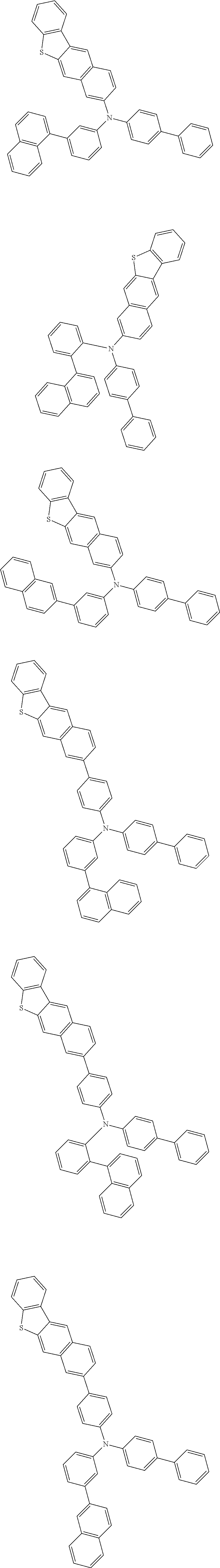 [SMILES: c1ccc(-c2ccc(N(c3cccc(-c4cccc5ccccc45)c3)c3ccc4cc5c(cc4c3)sc3ccccc35)cc2)cc1 ; c1ccc(-c2ccc(N(c3ccc4cc5c(cc4c3)sc3ccccc35)c3ccccc3-c3cccc4ccccc34)cc2)cc1 ; c1ccc(-c2ccc(N(c3cccc(-c4ccc5ccccc5c4)c3)c3ccc4cc5c(cc4c3)sc3ccccc35)cc2)cc1 ; c1ccc(-c2ccc(N(c3ccc(-c4ccc5cc6c(cc5c4)sc4ccccc46)cc3)c3cccc(-c4cccc5ccccc45)c3)cc2)cc1 ; c1ccc(-c2ccc(N(c3ccc(-c4ccc5cc6c(cc5c4)sc4ccccc46)cc3)c3ccccc3-c3cccc4ccccc34)cc2)cc1 ; c1ccc(-c2ccc(N(c3ccc(-c4ccc5cc6c(cc5c4)sc4ccccc46)cc3)c3cccc(-c4ccc5ccccc5c4)c3)cc2)cc1]

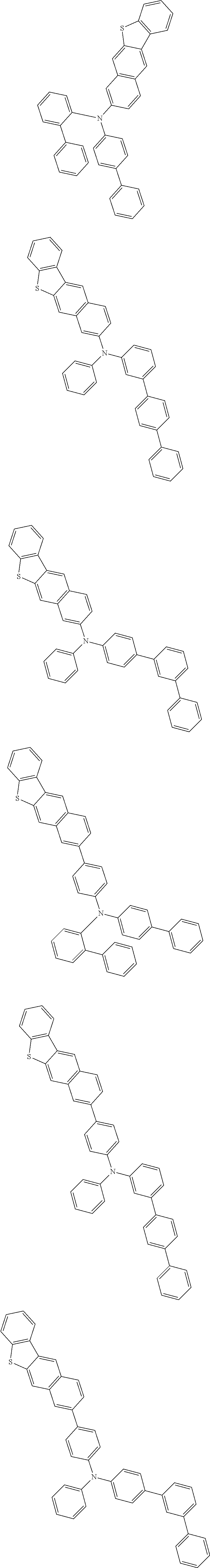 [SMILES: c1ccc(-c2ccc(N(c3ccc4cc5c(cc4c3)sc3ccccc35)c3ccccc3-c3ccccc3)cc2)cc1 ; c1ccc(-c2ccc(-c3cccc(N(c4ccccc4)c4ccc5cc6c(cc5c4)sc4ccccc46)c3)cc2)cc1 ; c1ccc(-c2cccc(-c3ccc(N(c4ccccc4)c4ccc5cc6c(cc5c4)sc4ccccc46)cc3)c2)cc1 ; c1ccc(-c2ccc(N(c3ccc(-c4ccc5cc6c(cc5c4)sc4ccccc46)cc3)c3ccccc3-c3ccccc3)cc2)cc1 ; c1ccc(-c2ccc(-c3cccc(N(c4ccccc4)c4ccc(-c5ccc6cc7c(cc6c5)sc5ccccc57)cc4)c3)cc2)cc1 ; c1ccc(-c2cccc(-c3ccc(N(c4ccccc4)c4ccc(-c5ccc6cc7c(cc6c5)sc5ccccc57)cc4)cc3)c2)cc1]

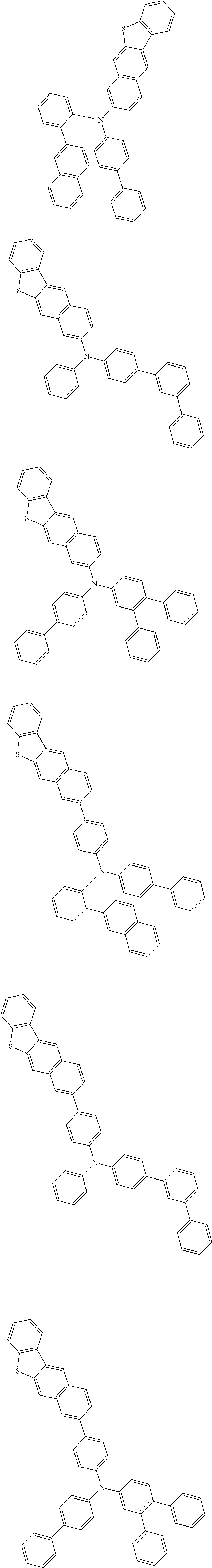 [SMILES: c1ccc(-c2ccc(N(c3ccc4cc5c(cc4c3)sc3ccccc35)c3ccccc3-c3ccc4ccccc4c3)cc2)cc1 ; c1ccc(-c2cccc(-c3ccc(N(c4ccccc4)c4ccc5cc6c(cc5c4)sc4ccccc46)cc3)c2)cc1 ; c1ccc(-c2ccc(N(c3ccc(-c4ccccc4)c(-c4ccccc4)c3)c3ccc4cc5c(cc4c3)sc3ccccc35)cc2)cc1 ; c1ccc(-c2ccc(N(c3ccc(-c4ccc5cc6c(cc5c4)sc4ccccc46)cc3)c3ccccc3-c3ccc4ccccc4c3)cc2)cc1 ; c1ccc(-c2cccc(-c3ccc(N(c4ccccc4)c4ccc(-c5ccc6cc7c(cc6c5)sc5ccccc57)cc4)cc3)c2)cc1 ; c1ccc(-c2ccc(N(c3ccc(-c4ccc5cc6c(cc5c4)sc4ccccc46)cc3)c3ccc(-c4ccccc4)c(-c4ccccc4)c3)cc2)cc1]

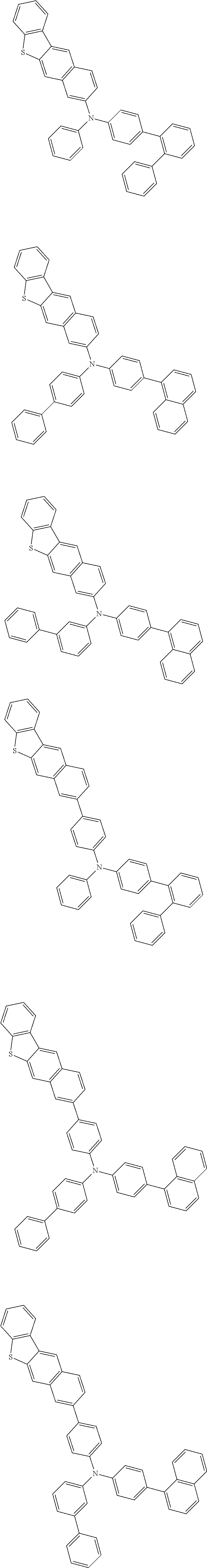 [SMILES: c1ccc(-c2ccccc2-c2ccc(N(c3ccccc3)c3ccc4cc5c(cc4c3)sc3ccccc35)cc2)cc1 ; c1ccc(-c2ccc(N(c3ccc(-c4cccc5ccccc45)cc3)c3ccc4cc5c(cc4c3)sc3ccccc35)cc2)cc1 ; c1ccc(-c2cccc(N(c3ccc(-c4cccc5ccccc45)cc3)c3ccc4cc5c(cc4c3)sc3ccccc35)c2)cc1 ; c1ccc(-c2ccccc2-c2ccc(N(c3ccccc3)c3ccc(-c4ccc5cc6c(cc5c4)sc4ccccc46)cc3)cc2)cc1 ; c1ccc(-c2ccc(N(c3ccc(-c4ccc5cc6c(cc5c4)sc4ccccc46)cc3)c3ccc(-c4cccc5ccccc45)cc3)cc2)cc1 ; c1ccc(-c2cccc(N(c3ccc(-c4ccc5cc6c(cc5c4)sc4ccccc46)cc3)c3ccc(-c4cccc5ccccc45)cc3)c2)cc1]

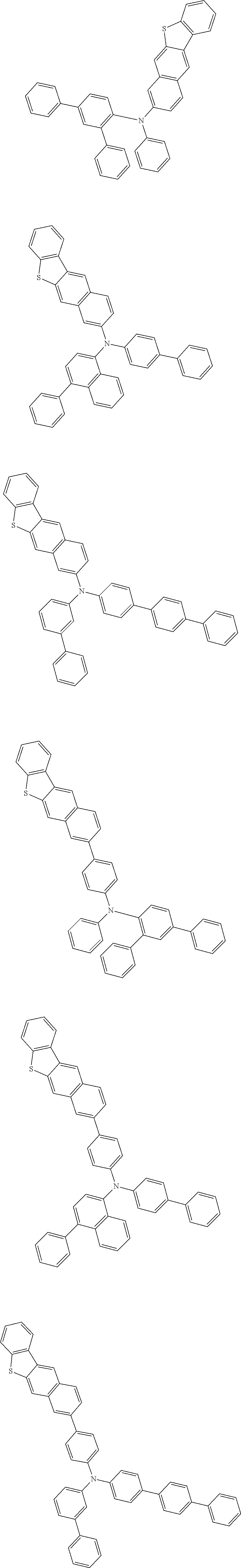

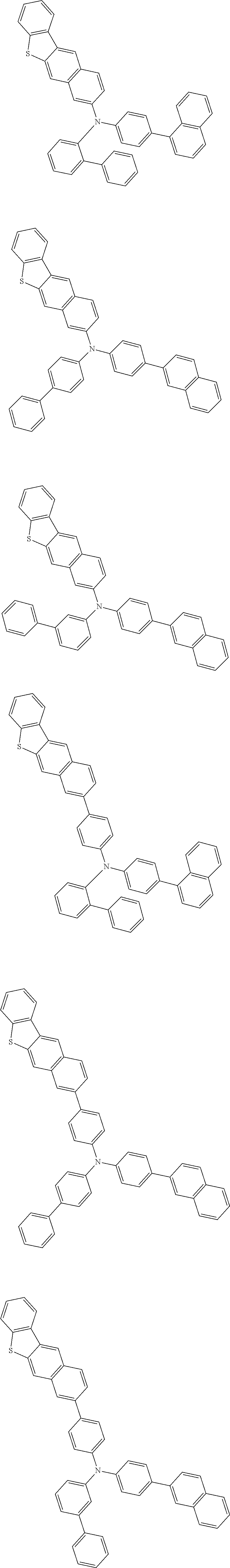 [SMILES: c1ccc(-c2ccccc2N(c2ccc(-c3cccc4ccccc34)cc2)c2ccc3cc4c(cc3c2)sc2ccccc24)cc1 ; c1ccc(-c2ccc(N(c3ccc(-c4ccc5ccccc5c4)cc3)c3ccc4cc5c(cc4c3)sc3ccccc35)cc2)cc1 ; c1ccc(-c2cccc(N(c3ccc(-c4ccc5ccccc5c4)cc3)c3ccc4cc5c(cc4c3)sc3ccccc35)c2)cc1 ; c1ccc(-c2ccccc2N(c2ccc(-c3ccc4cc5c(cc4c3)sc3ccccc35)cc2)c2ccc(-c3cccc4ccccc34)cc2)cc1 ; c1ccc(-c2ccc(N(c3ccc(-c4ccc5ccccc5c4)cc3)c3ccc(-c4ccc5cc6c(cc5c4)sc4ccccc46)cc3)cc2)cc1 ; c1ccc(-c2cccc(N(c3ccc(-c4ccc5ccccc5c4)cc3)c3ccc(-c4ccc5cc6c(cc5c4)sc4ccccc46)cc3)c2)cc1]

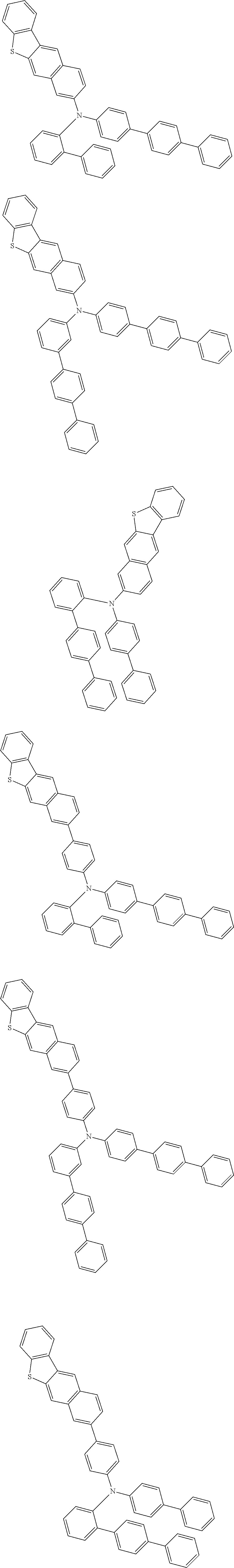

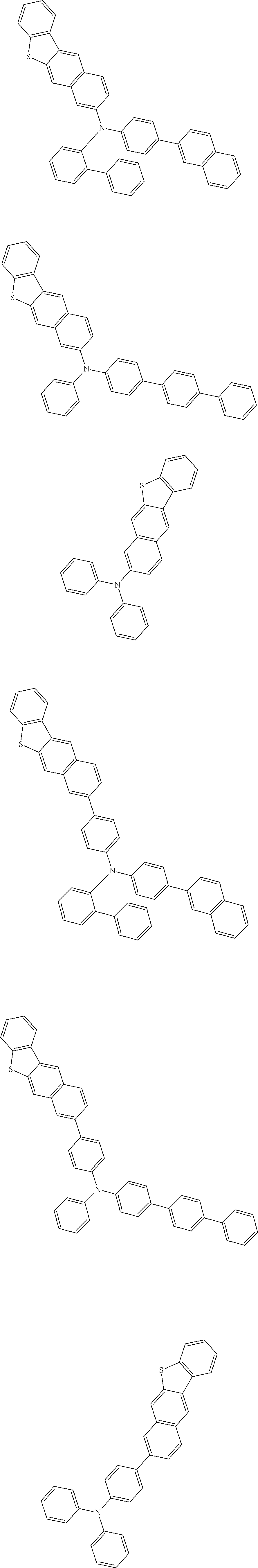 [SMILES: c1ccc(-c2ccccc2N(c2ccc(-c3ccc4ccccc4c3)cc2)c2ccc3cc4c(cc3c2)sc2ccccc24)cc1 ; c1ccc(-c2ccc(-c3ccc(N(c4ccccc4)c4ccc5cc6c(cc5c4)sc4ccccc46)cc3)cc2)cc1 ; c1ccc(N(c2ccccc2)c2ccc3cc4c(cc3c2)sc2ccccc24)cc1 ; c1ccc(-c2ccccc2N(c2ccc(-c3ccc4ccccc4c3)cc2)c2ccc(-c3ccc4cc5c(cc4c3)sc3ccccc35)cc2)cc1 ; c1ccc(-c2ccc(-c3ccc(N(c4ccccc4)c4ccc(-c5ccc6cc7c(cc6c5)sc5ccccc57)cc4)cc3)cc2)cc1 ; c1ccc(N(c2ccccc2)c2ccc(-c3ccc4cc5c(cc4c3)sc3ccccc35)cc2)cc1]

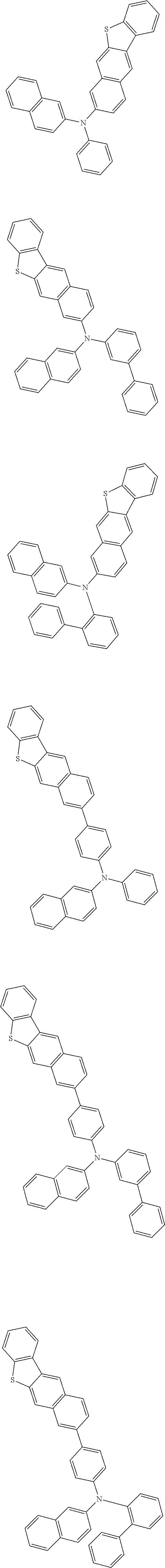 [SMILES: c1ccc(N(c2ccc3ccccc3c2)c2ccc3cc4c(cc3c2)sc2ccccc24)cc1 ; c1ccc(-c2cccc(N(c3ccc4ccccc4c3)c3ccc4cc5c(cc4c3)sc3ccccc35)c2)cc1 ; c1ccc(-c2ccccc2N(c2ccc3ccccc3c2)c2ccc3cc4c(cc3c2)sc2ccccc24)cc1 ; c1ccc(N(c2ccc(-c3ccc4cc5c(cc4c3)sc3ccccc35)cc2)c2ccc3ccccc3c2)cc1 ; c1ccc(-c2cccc(N(c3ccc(-c4ccc5cc6c(cc5c4)sc4ccccc46)cc3)c3ccc4ccccc4c3)c2)cc1 ; c1ccc(-c2ccccc2N(c2ccc(-c3ccc4cc5c(cc4c3)sc3ccccc35)cc2)c2ccc3ccccc3c2)cc1]

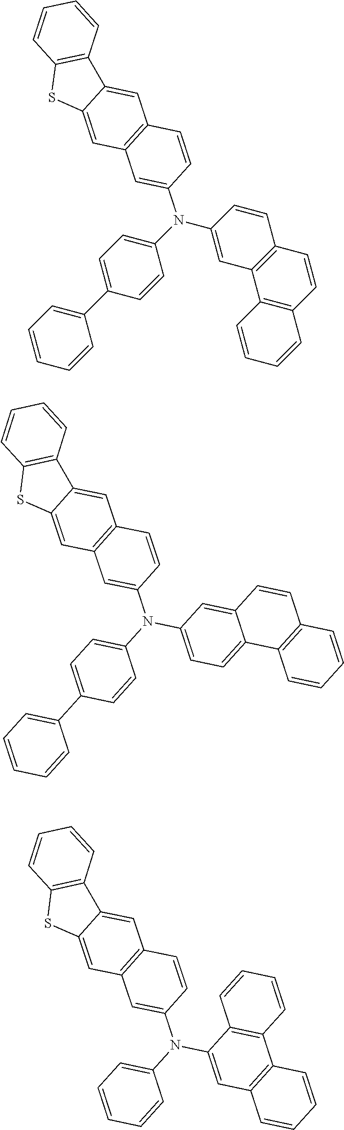

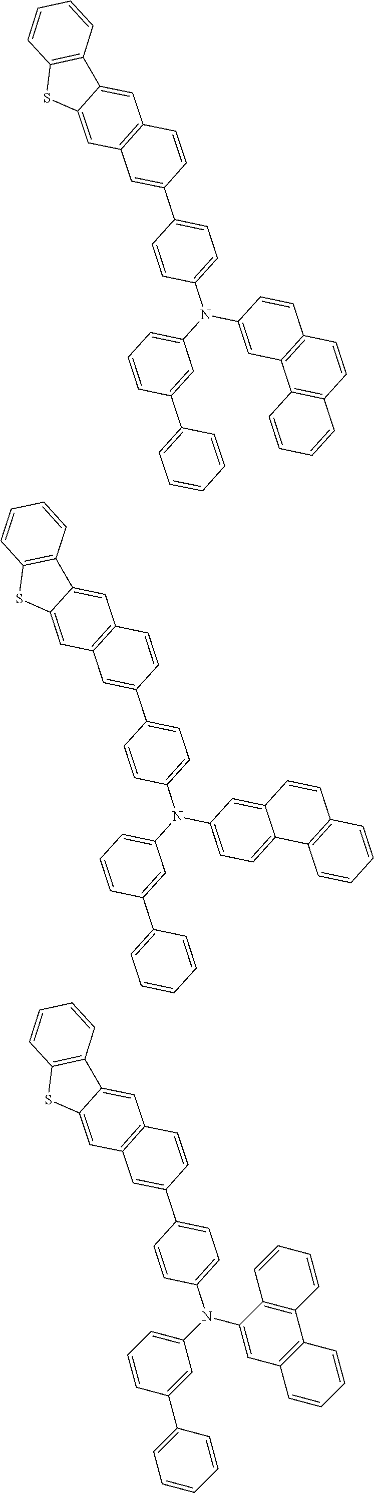 [SMILES: c1ccc(-c2cccc(N(c3ccc(-c4ccc5cc6c(cc5c4)sc4ccccc46)cc3)c3ccc4ccc5ccccc5c4c3)c2)cc1 ; c1ccc(-c2cccc(N(c3ccc(-c4ccc5cc6c(cc5c4)sc4ccccc46)cc3)c3ccc4c(ccc5ccccc54)c3)c2)cc1 ; c1ccc(-c2cccc(N(c3ccc(-c4ccc5cc6c(cc5c4)sc4ccccc46)cc3)c3cc4ccccc4c4ccccc34)c2)cc1]

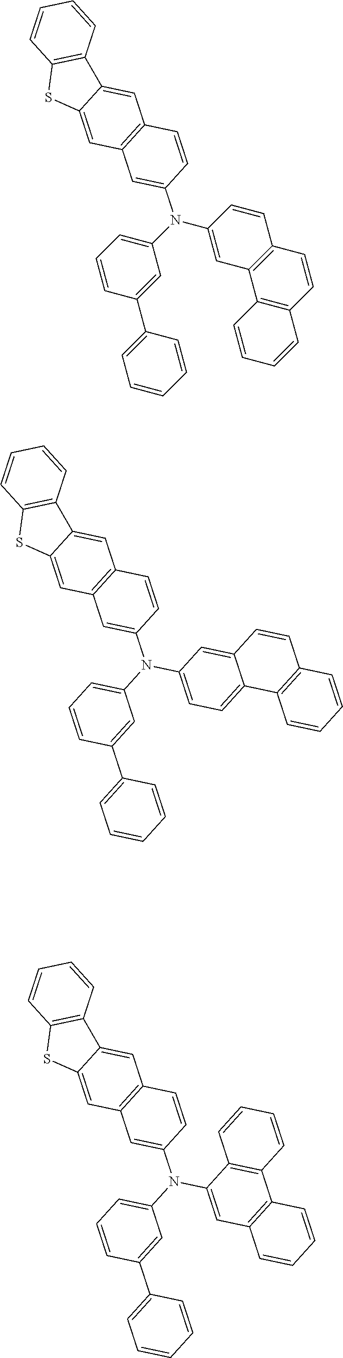 [SMILES: c1ccc(-c2cccc(N(c3ccc4cc5c(cc4c3)sc3ccccc35)c3ccc4ccc5ccccc5c4c3)c2)cc1 ; c1ccc(-c2cccc(N(c3ccc4cc5c(cc4c3)sc3ccccc35)c3ccc4c(ccc5ccccc54)c3)c2)cc1 ; c1ccc(-c2cccc(N(c3ccc4cc5c(cc4c3)sc3ccccc35)c3cc4ccccc4c4ccccc34)c2)cc1]

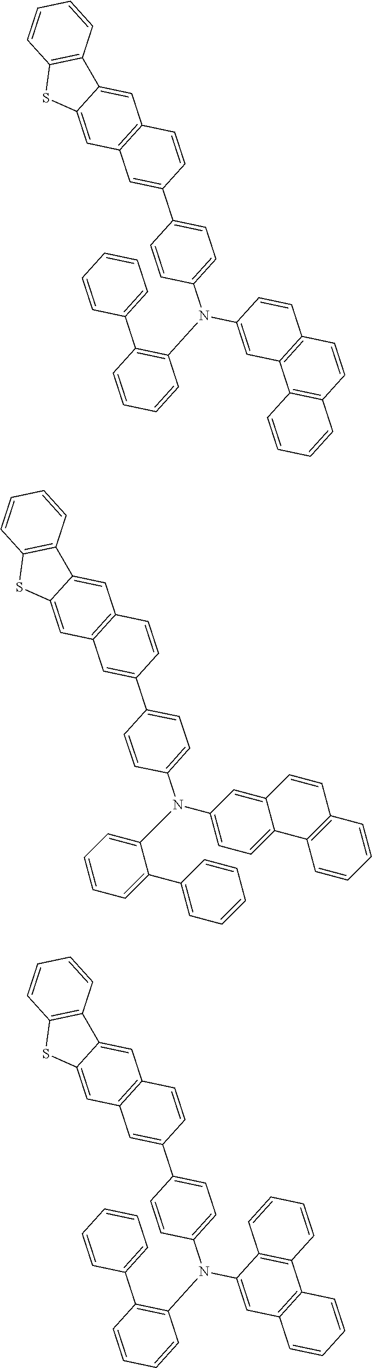 [SMILES: c1ccc(-c2ccccc2N(c2ccc(-c3ccc4cc5c(cc4c3)sc3ccccc35)cc2)c2ccc3ccc4ccccc4c3c2)cc1 ; c1ccc(-c2ccccc2N(c2ccc(-c3ccc4cc5c(cc4c3)sc3ccccc35)cc2)c2ccc3c(ccc4ccccc43)c2)cc1 ; c1ccc(-c2ccccc2N(c2ccc(-c3ccc4cc5c(cc4c3)sc3ccccc35)cc2)c2cc3ccccc3c3ccccc23)cc1]

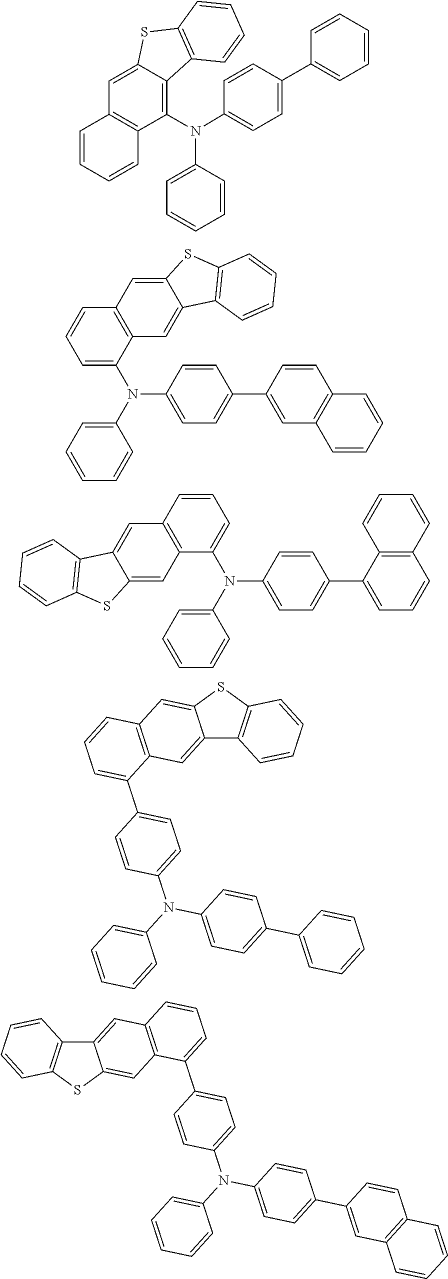

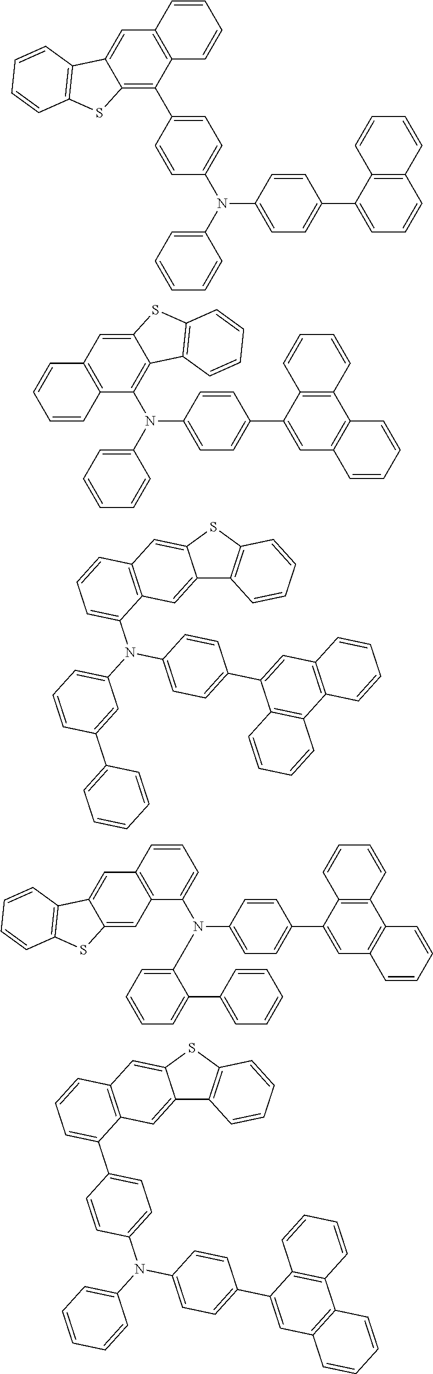

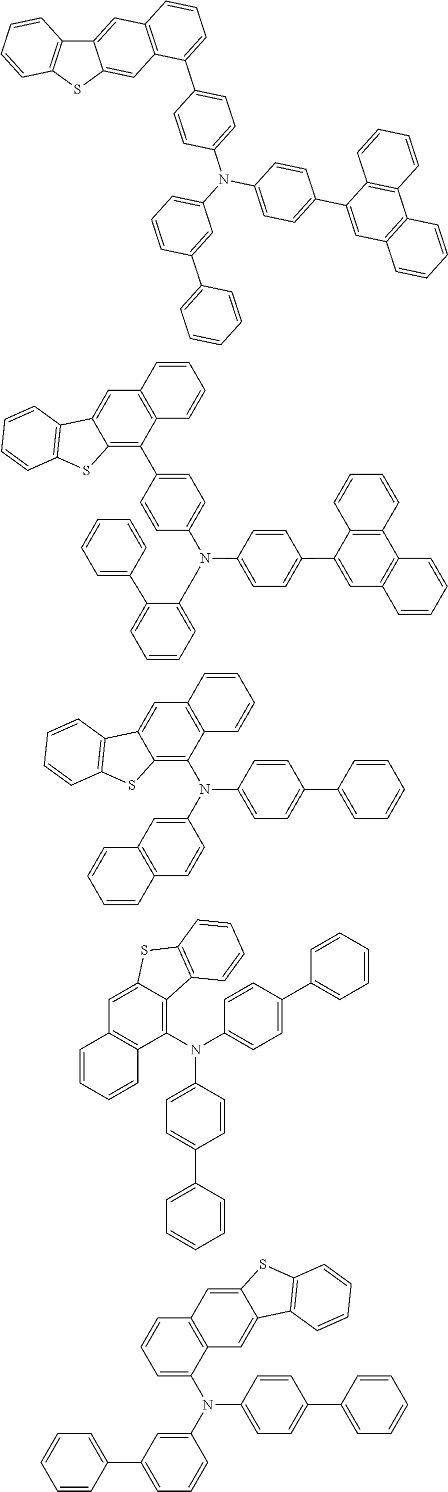

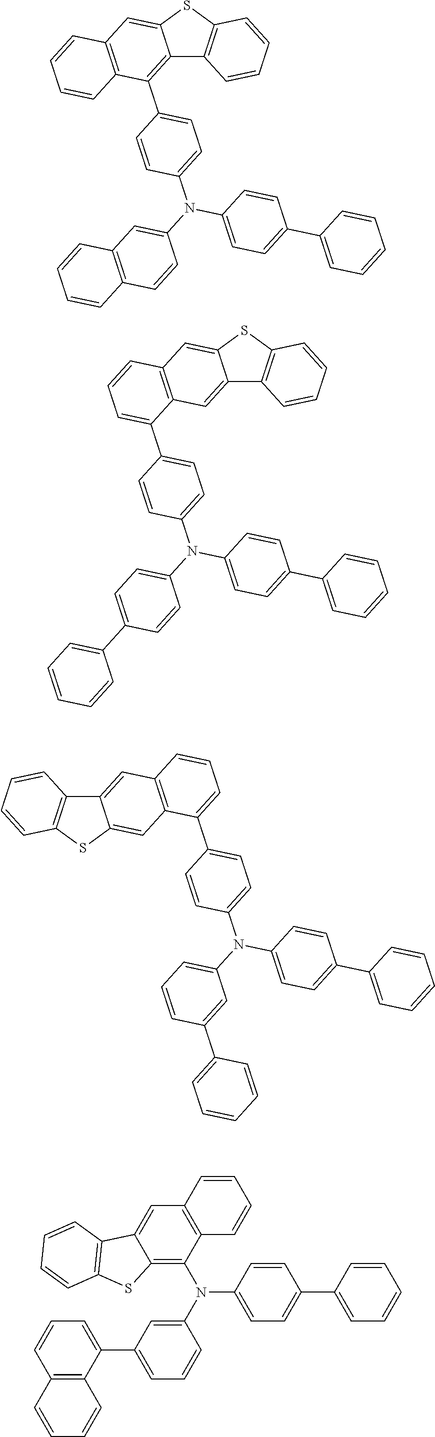 [SMILES: c1ccc(-c2ccc(N(c3ccc(-c4c5ccccc5cc5sc6ccccc6c45)cc3)c3ccc4ccccc4c3)cc2)cc1 ; c1ccc(-c2ccc(N(c3ccc(-c4ccccc4)cc3)c3ccc(-c4cccc5cc6sc7ccccc7c6cc45)cc3)cc2)cc1 ; c1ccc(-c2ccc(N(c3ccc(-c4cccc5cc6c(cc45)sc4ccccc46)cc3)c3cccc(-c4ccccc4)c3)cc2)cc1 ; c1ccc(-c2ccc(N(c3cccc(-c4cccc5ccccc45)c3)c3c4ccccc4cc4c3sc3ccccc34)cc2)cc1]

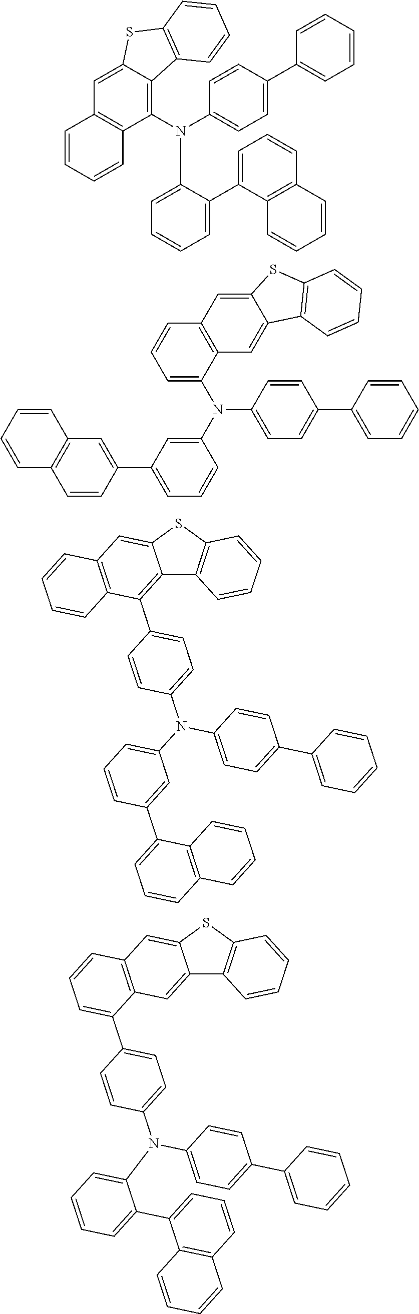

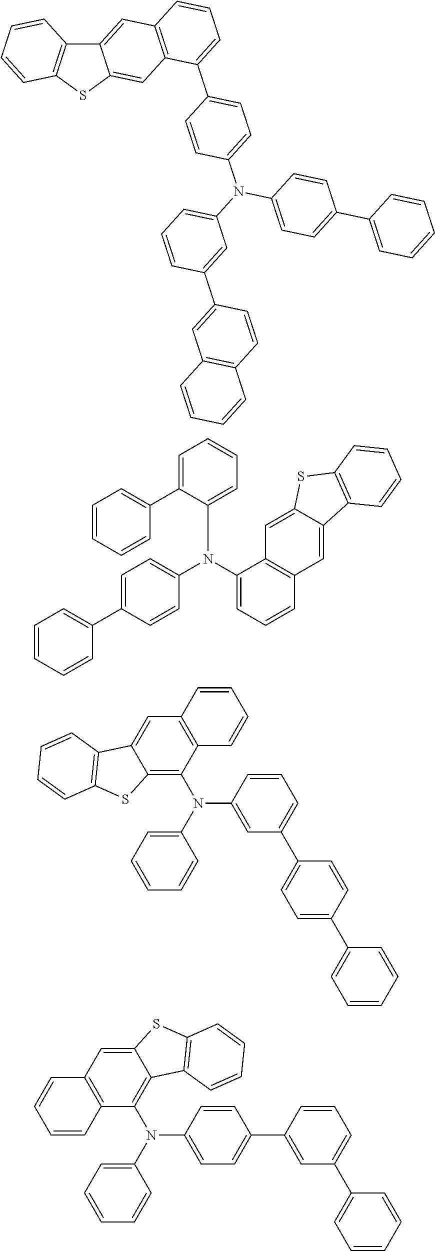

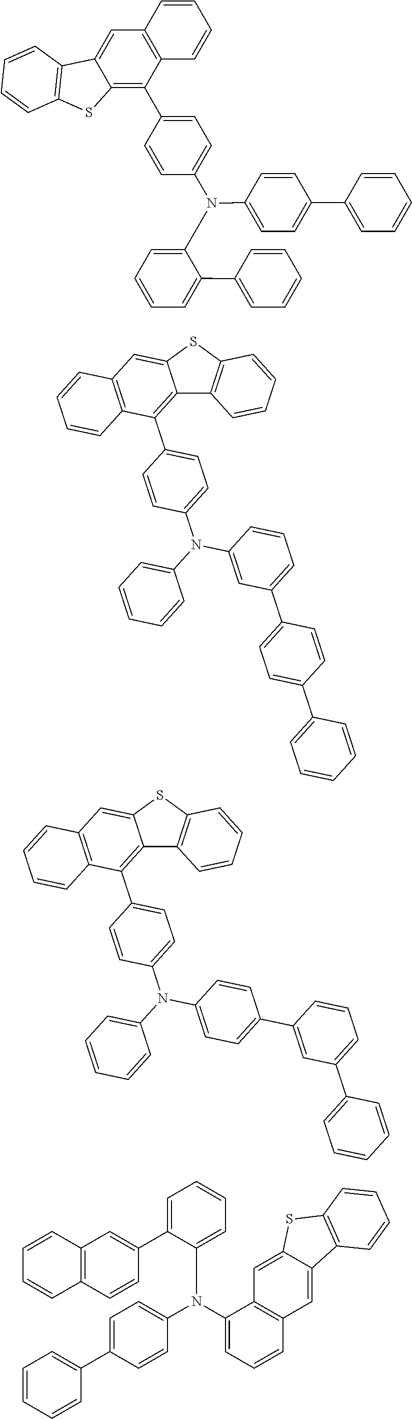

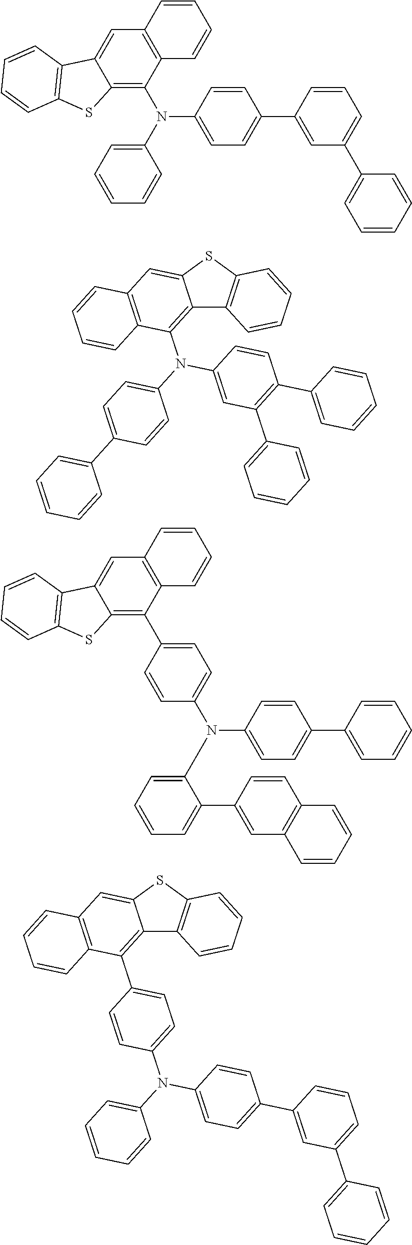

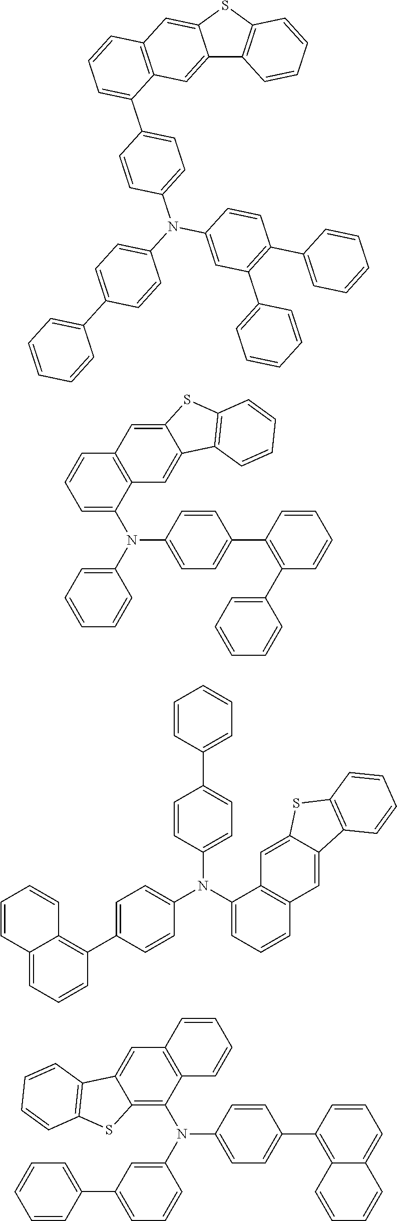

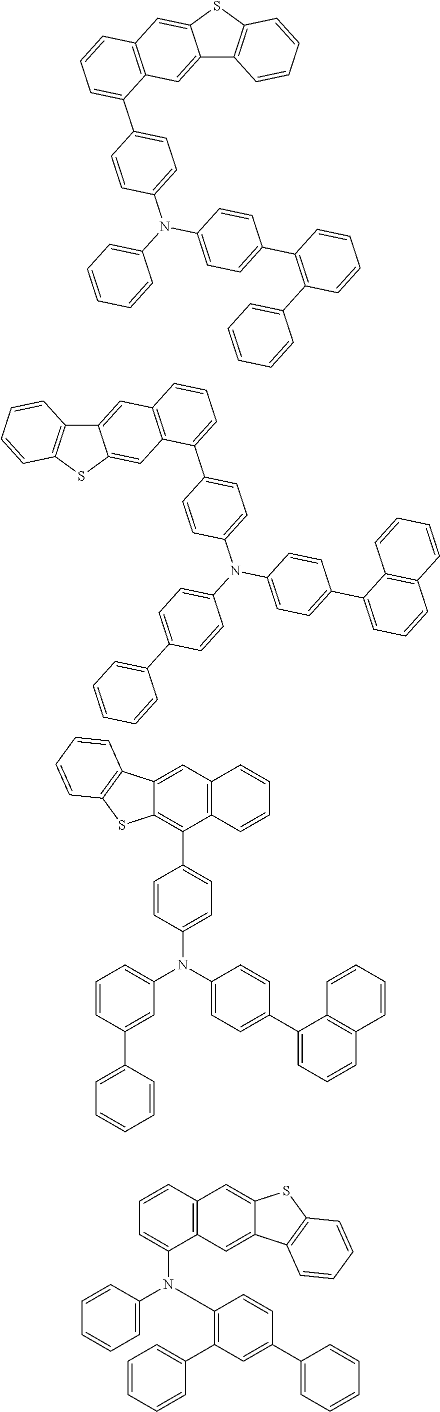

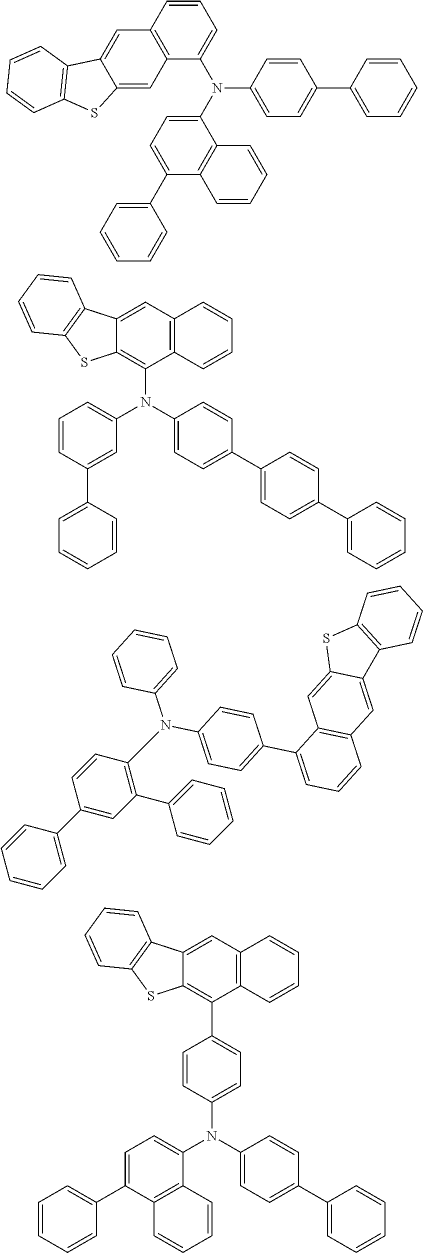

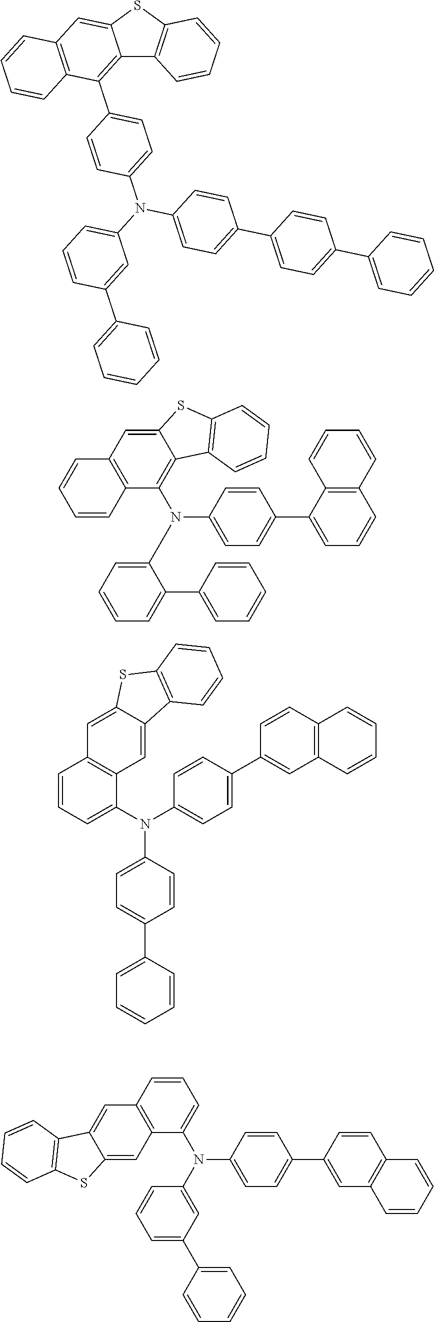

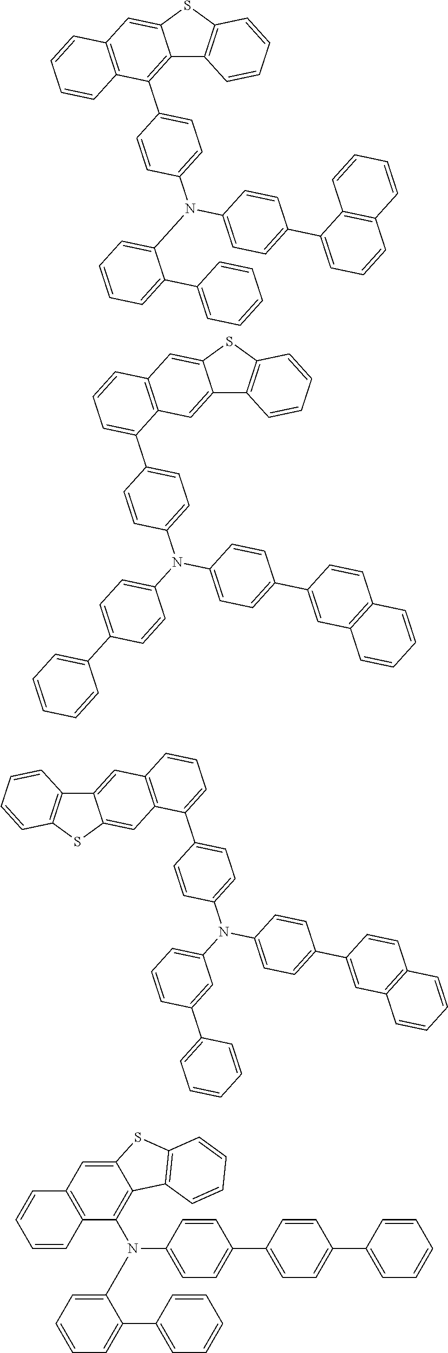

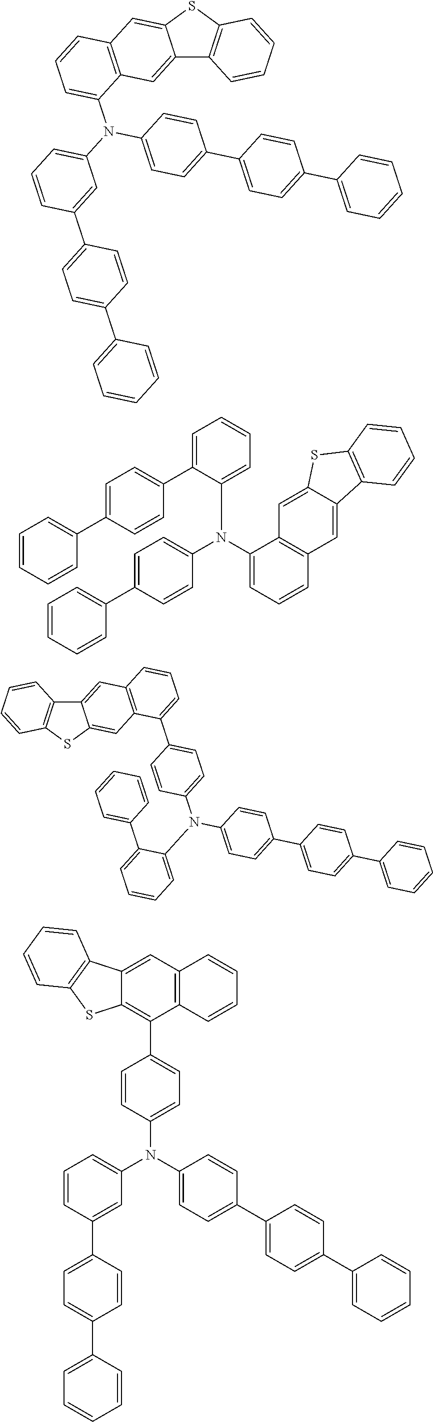

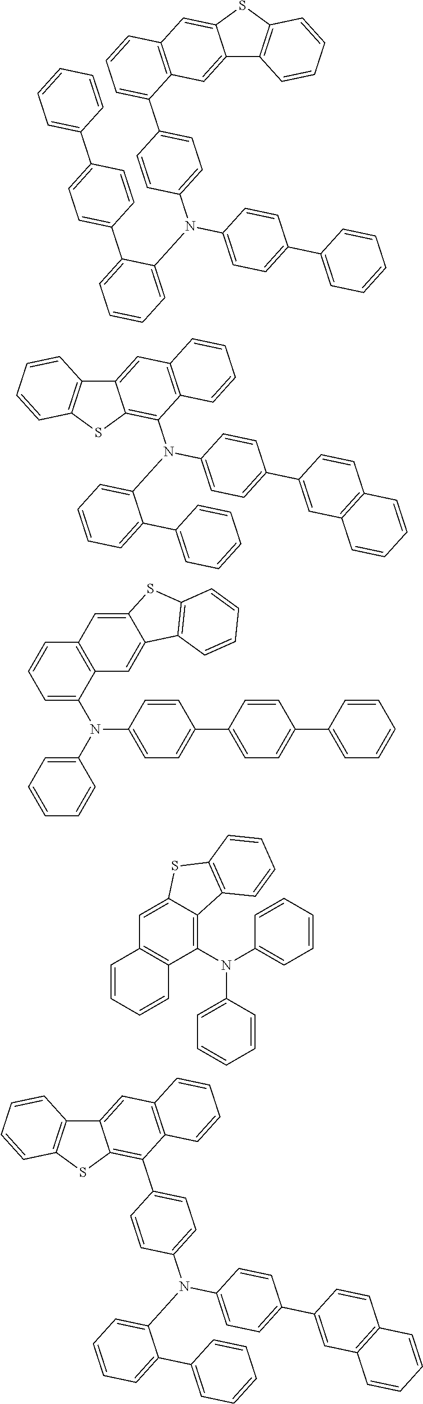

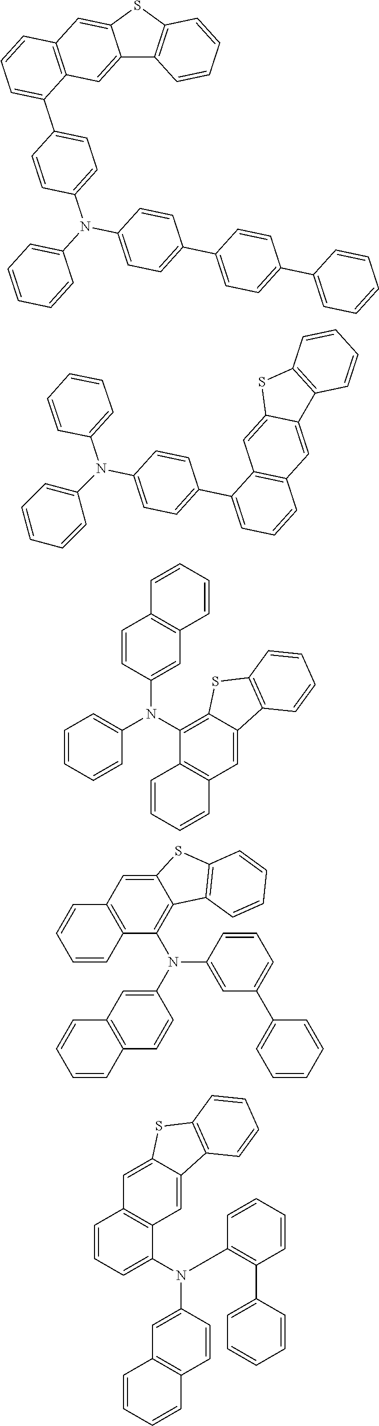

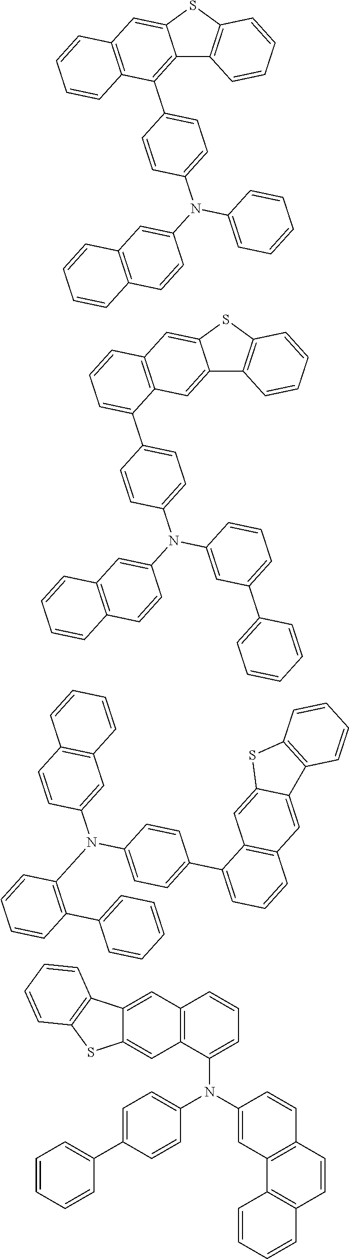

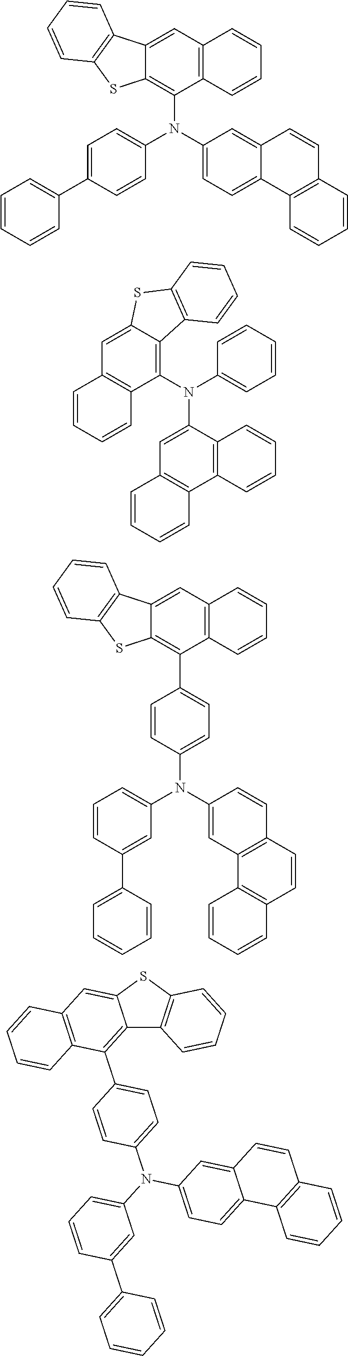

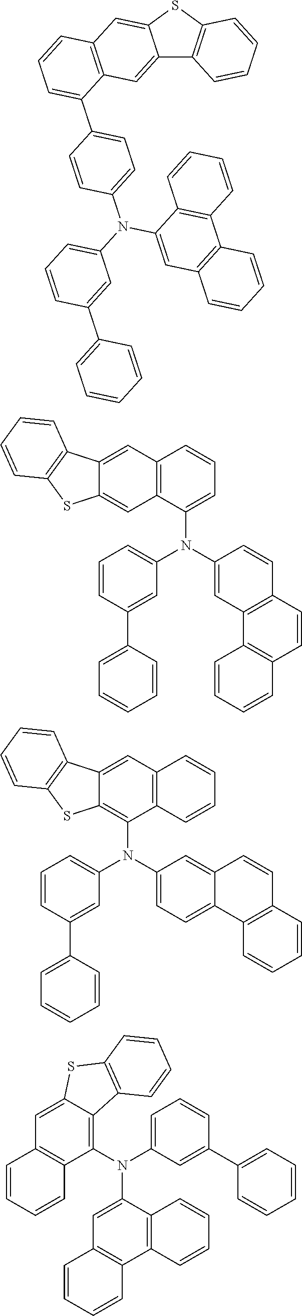

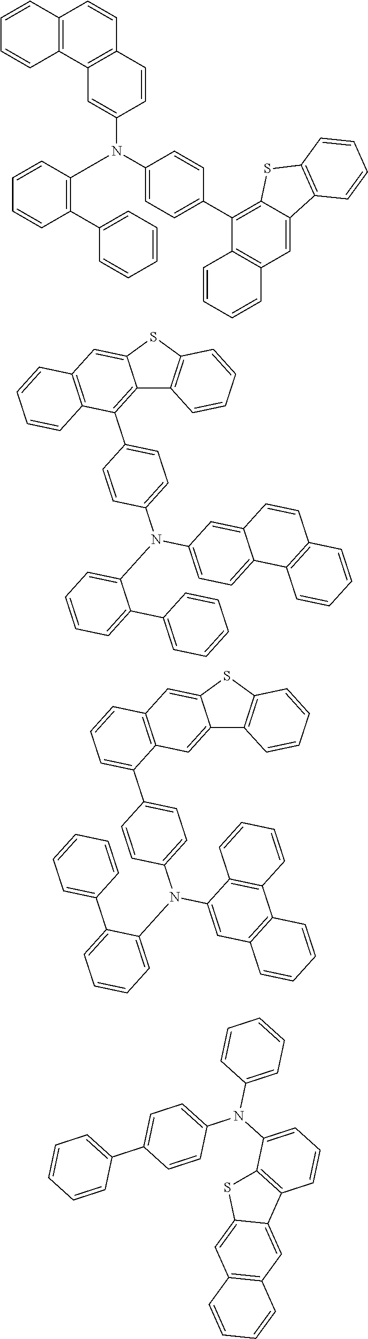

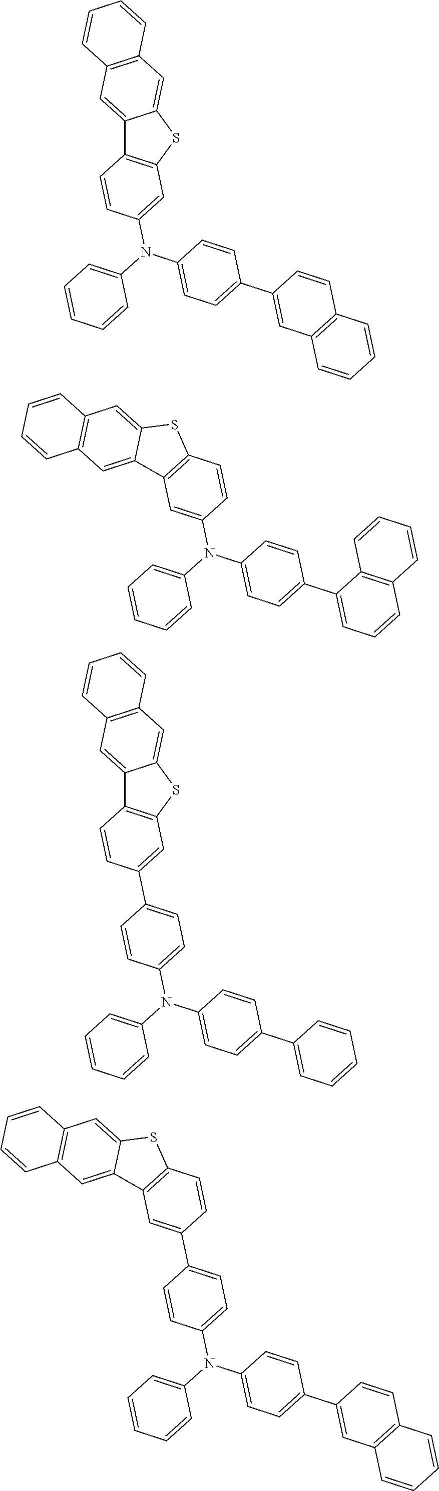

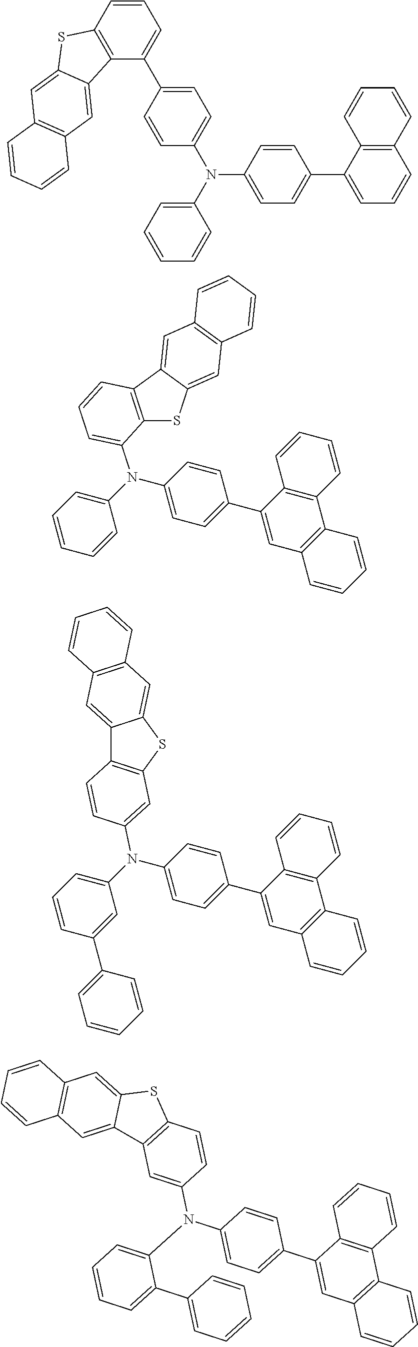

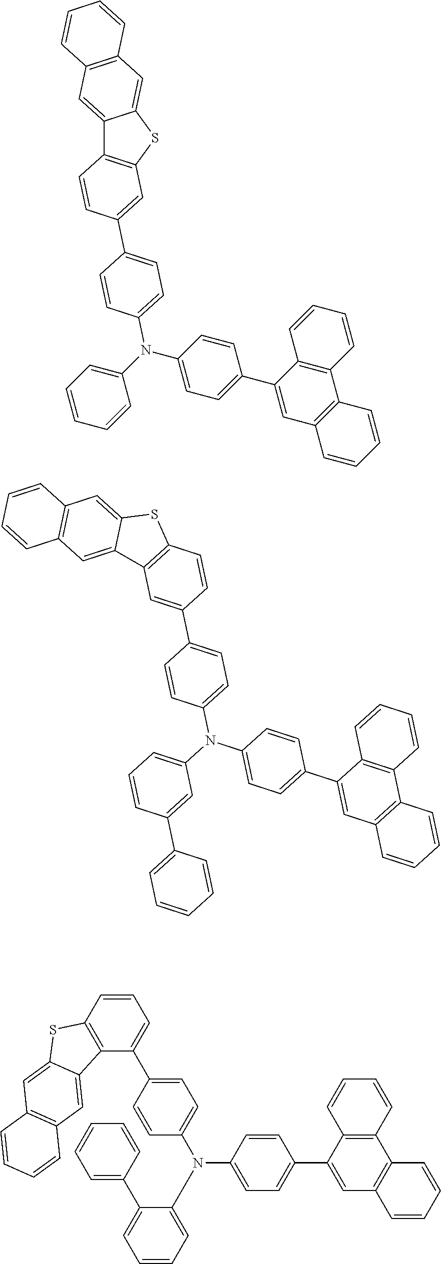 [SMILES: c1ccc(N(c2ccc(-c3ccc4c(c3)sc3cc5ccccc5cc34)cc2)c2ccc(-c3cc4ccccc4c4ccccc34)cc2)cc1 ; c1ccc(-c2cccc(N(c3ccc(-c4ccc5sc6cc7ccccc7cc6c5c4)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cccc4sc5cc6ccccc6cc5c34)cc2)cc1]

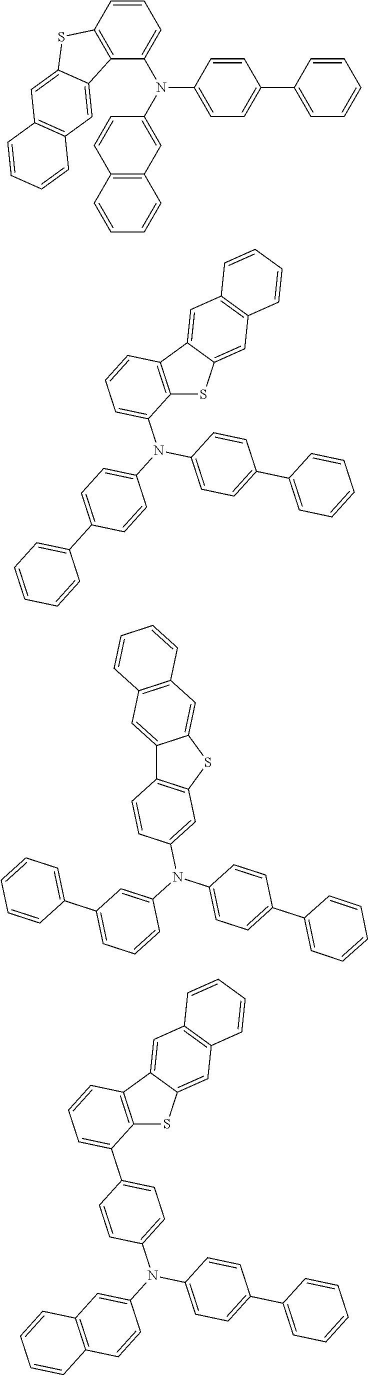 [SMILES: c1ccc(-c2ccc(N(c3ccc4ccccc4c3)c3cccc4sc5cc6ccccc6cc5c34)cc2)cc1 ; c1ccc(-c2ccc(N(c3ccc(-c4ccccc4)cc3)c3cccc4c3sc3cc5ccccc5cc34)cc2)cc1 ; c1ccc(-c2ccc(N(c3cccc(-c4ccccc4)c3)c3ccc4c(c3)sc3cc5ccccc5cc34)cc2)cc1 ; c1ccc(-c2ccc(N(c3ccc(-c4cccc5c4sc4cc6ccccc6cc45)cc3)c3ccc4ccccc4c3)cc2)cc1]

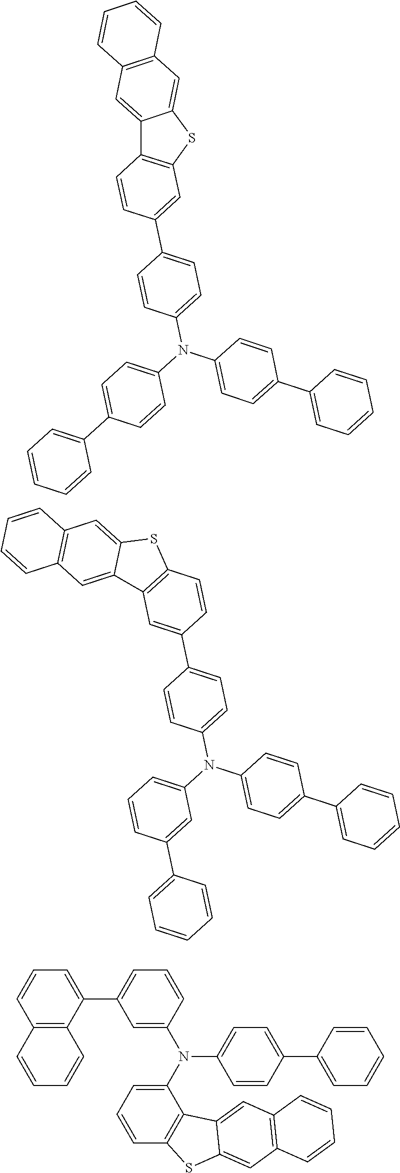

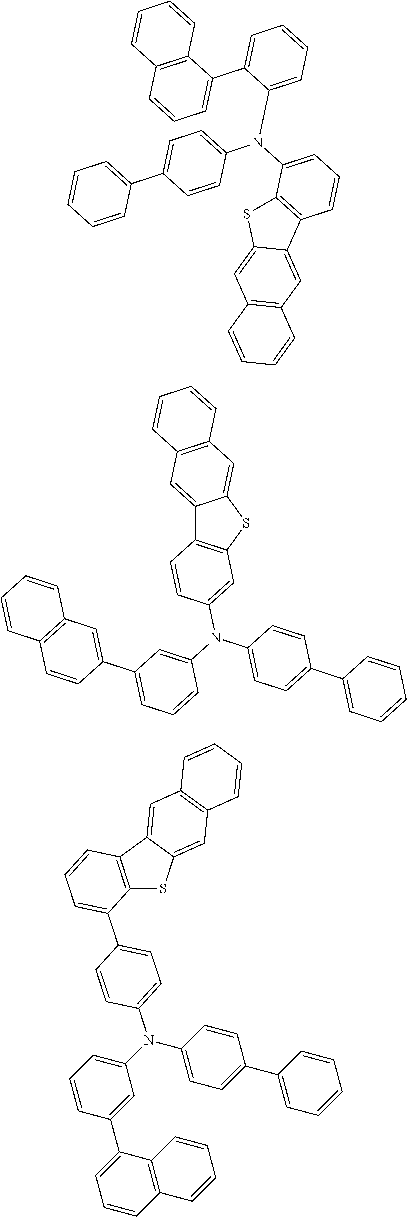

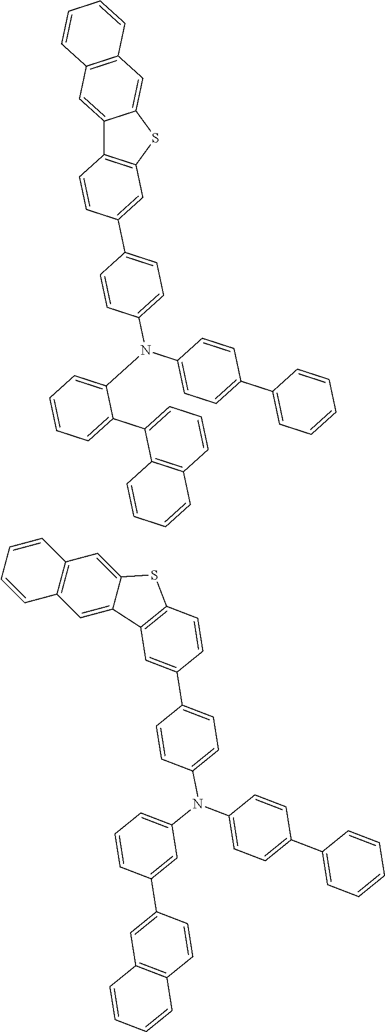 [SMILES: c1ccc(-c2ccc(N(c3ccc(-c4ccc5c(c4)sc4cc6ccccc6cc45)cc3)c3ccccc3-c3cccc4ccccc34)cc2)cc1 ; c1ccc(-c2ccc(N(c3ccc(-c4ccc5sc6cc7ccccc7cc6c5c4)cc3)c3cccc(-c4ccc5ccccc5c4)c3)cc2)cc1]

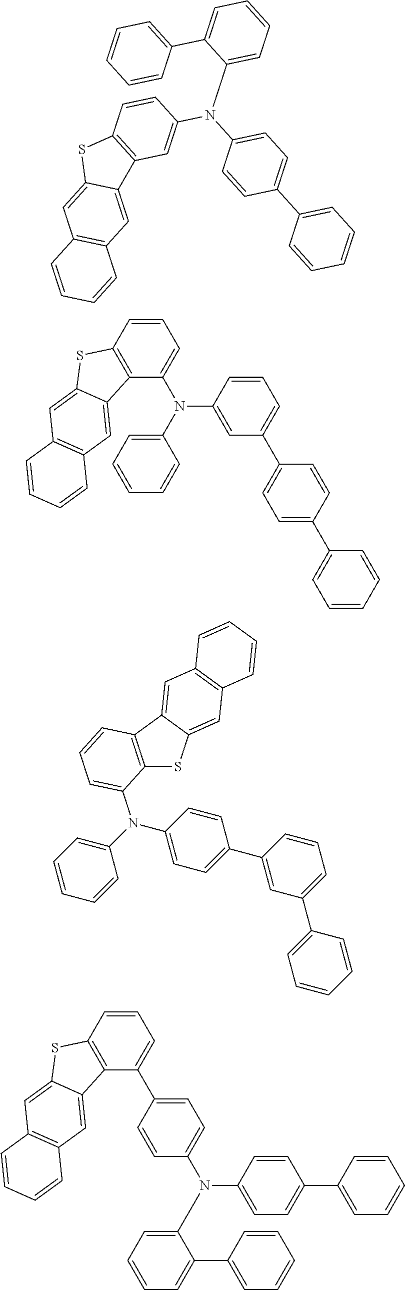

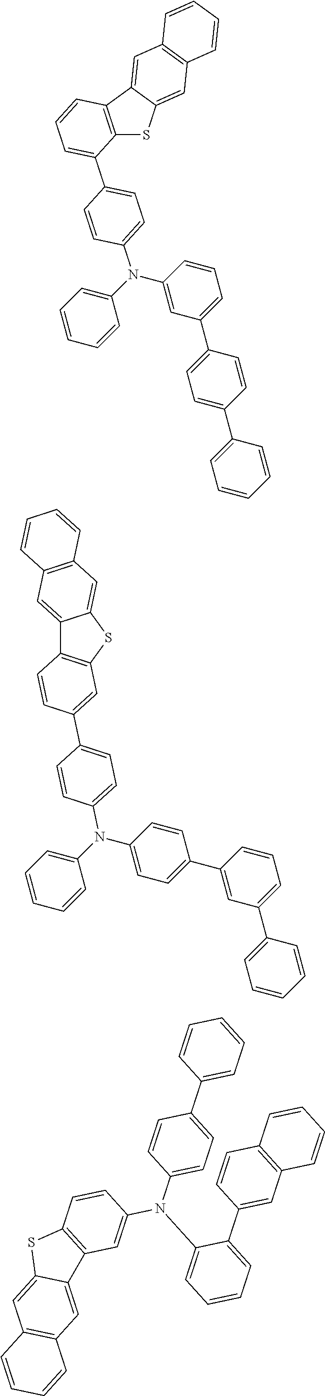

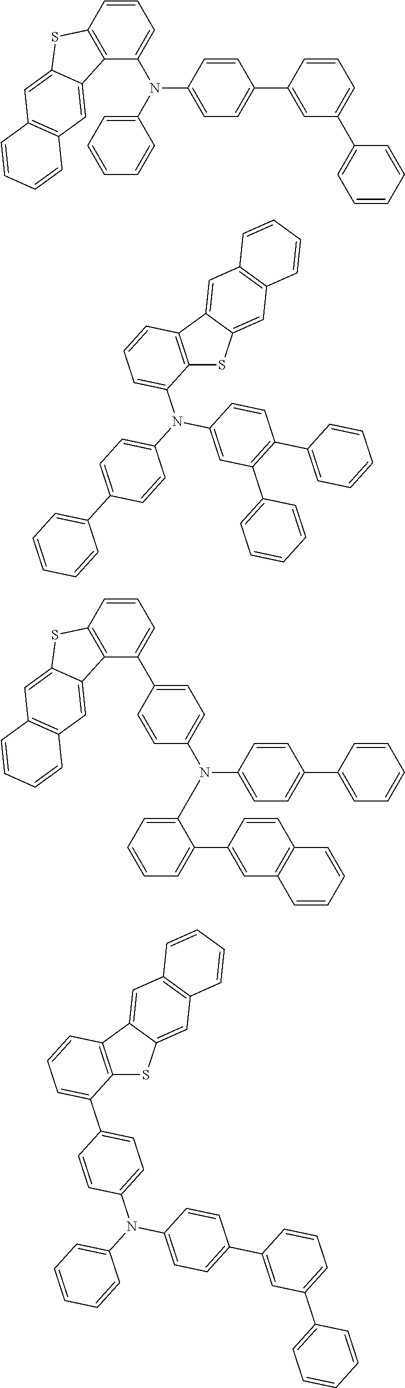

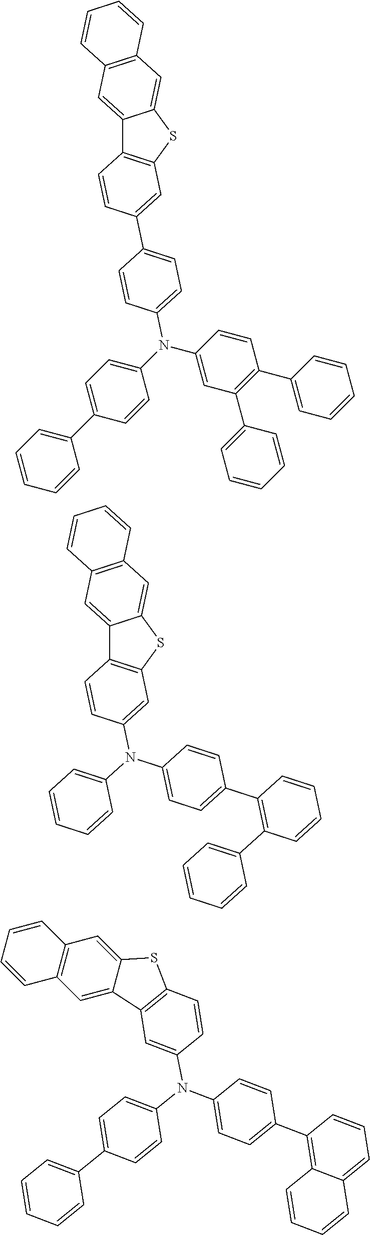

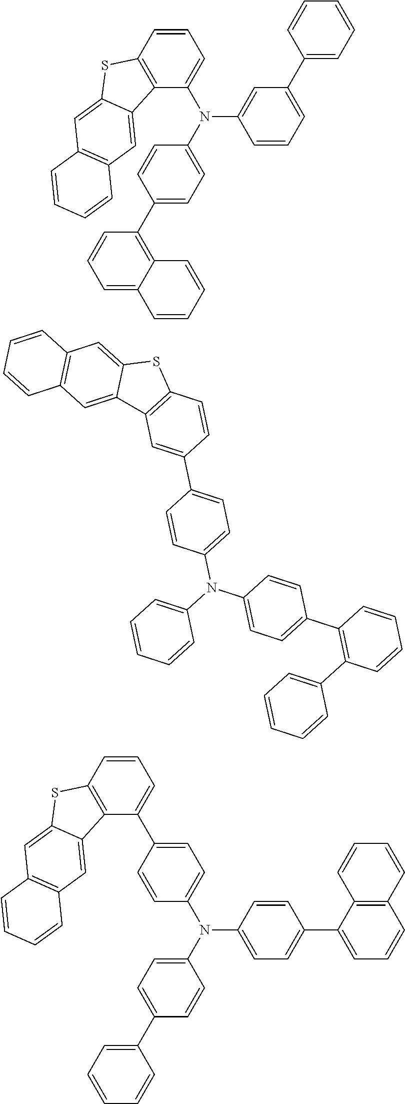

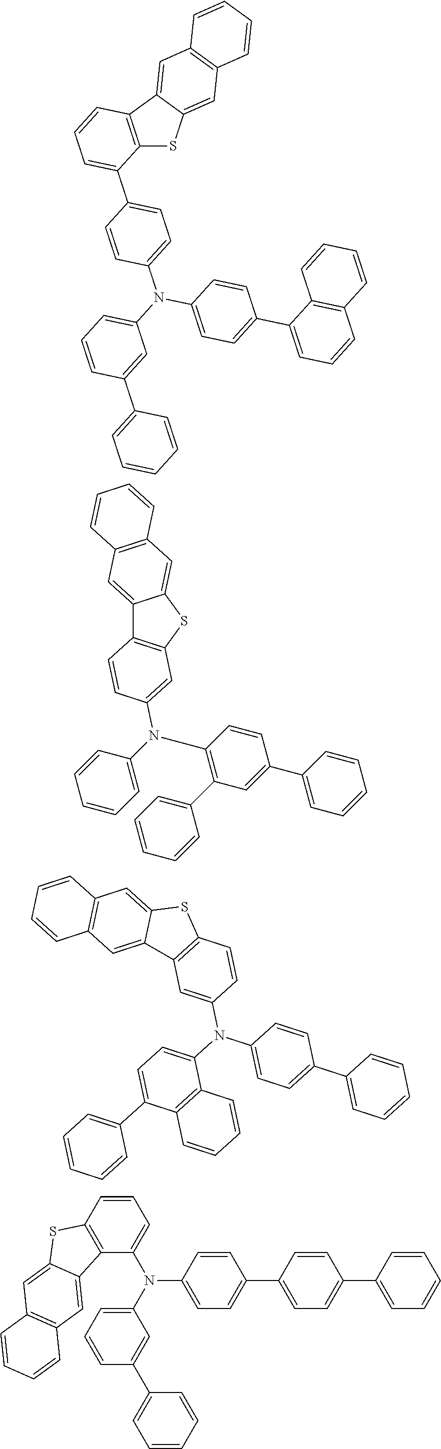

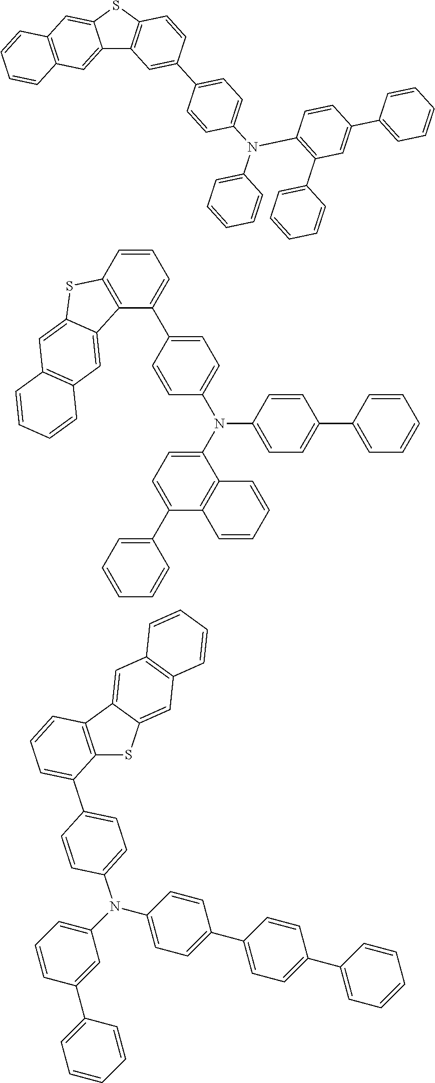

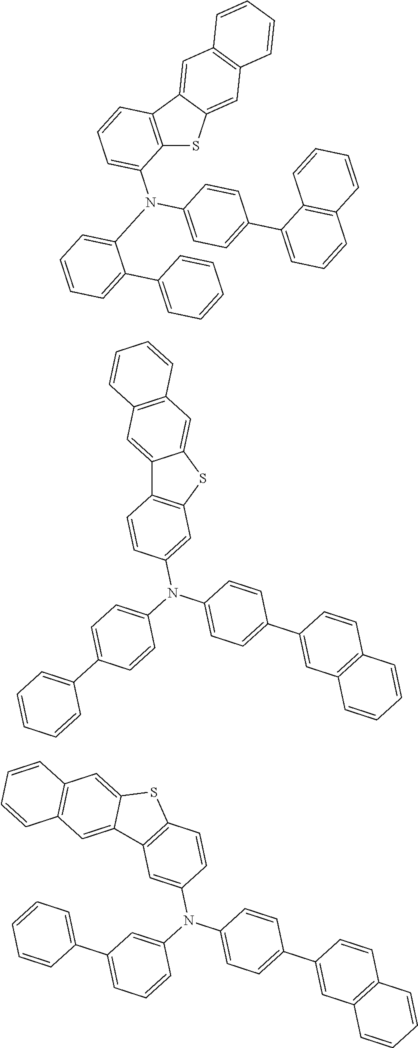

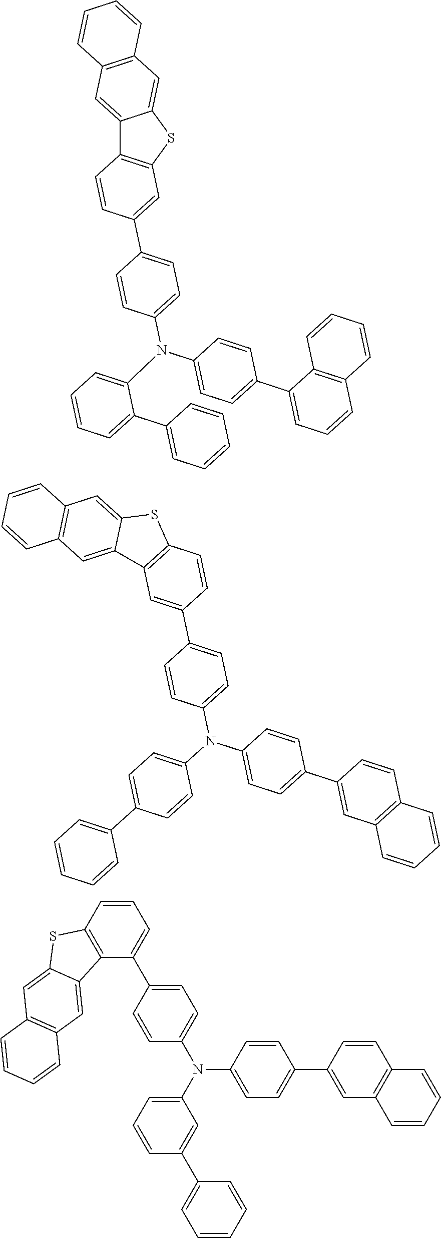

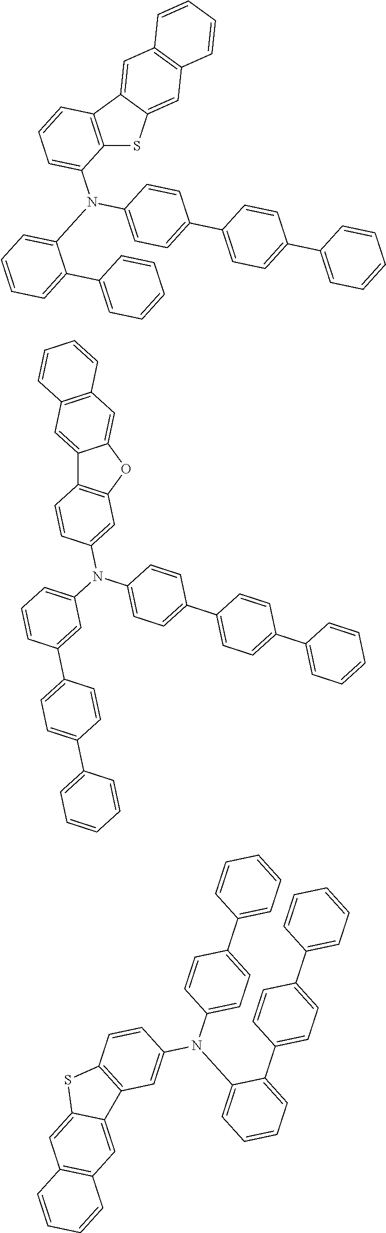

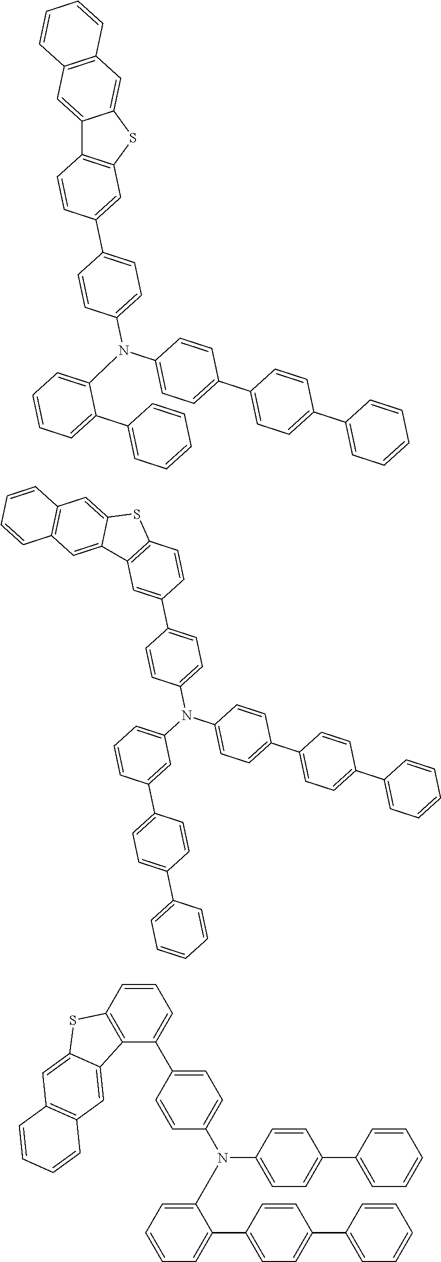

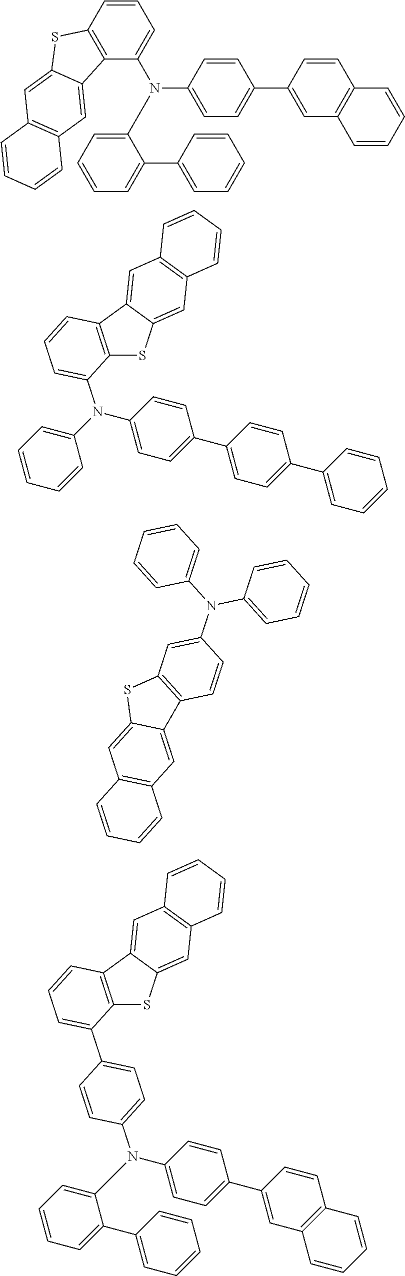

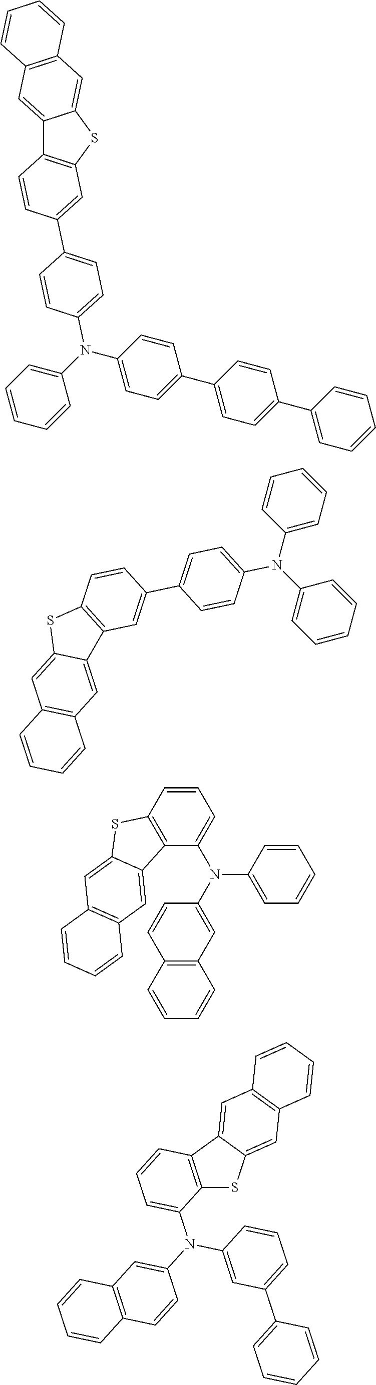

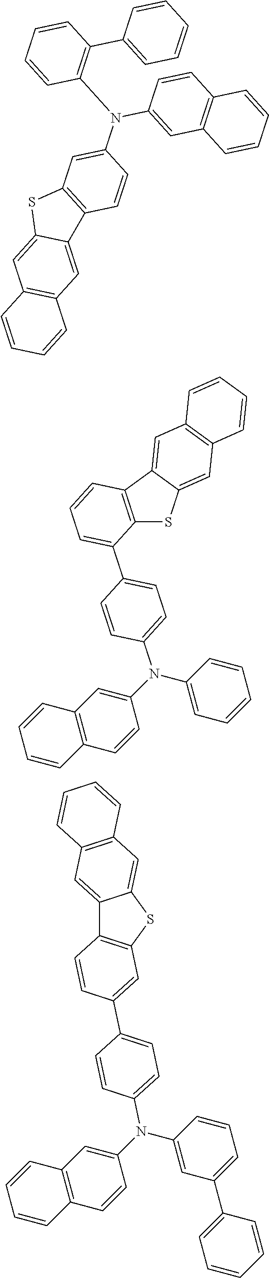

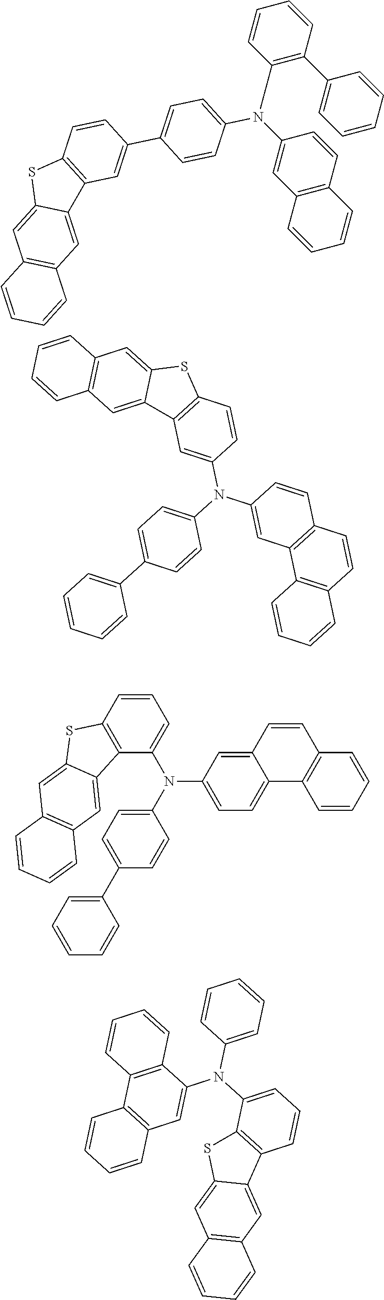

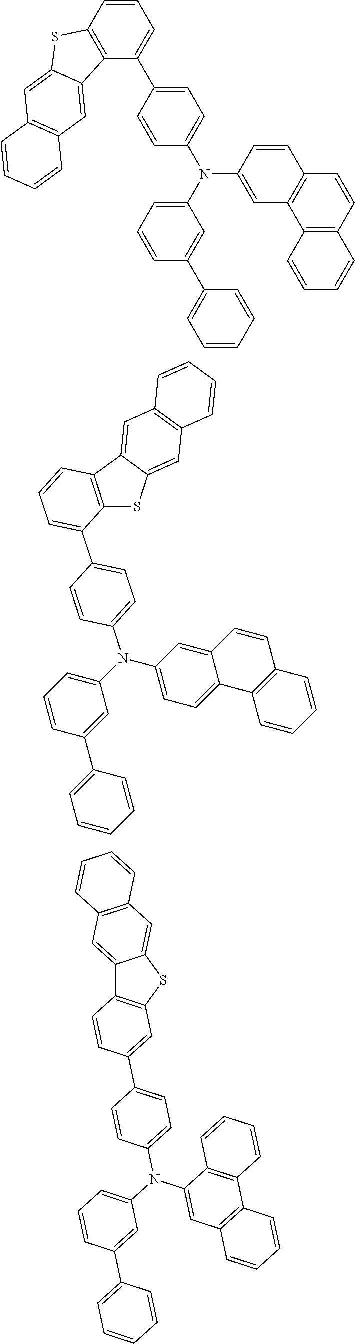

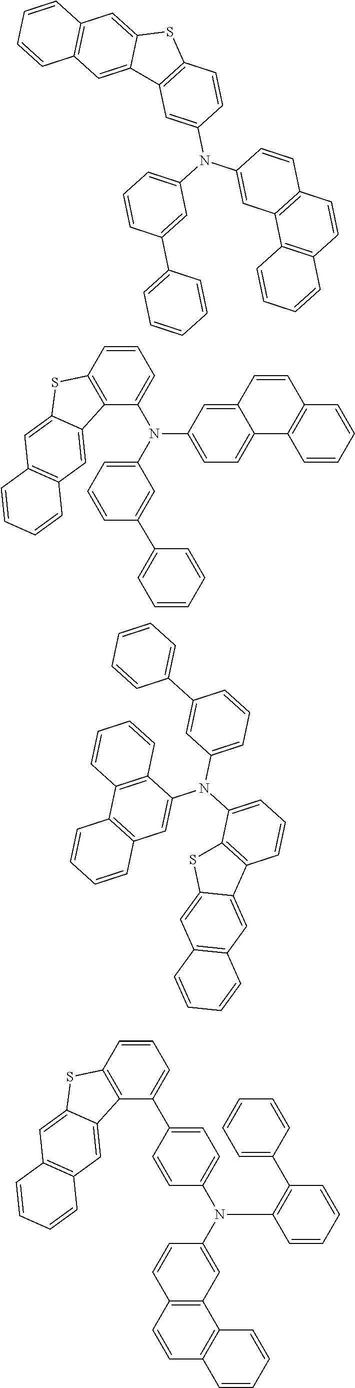

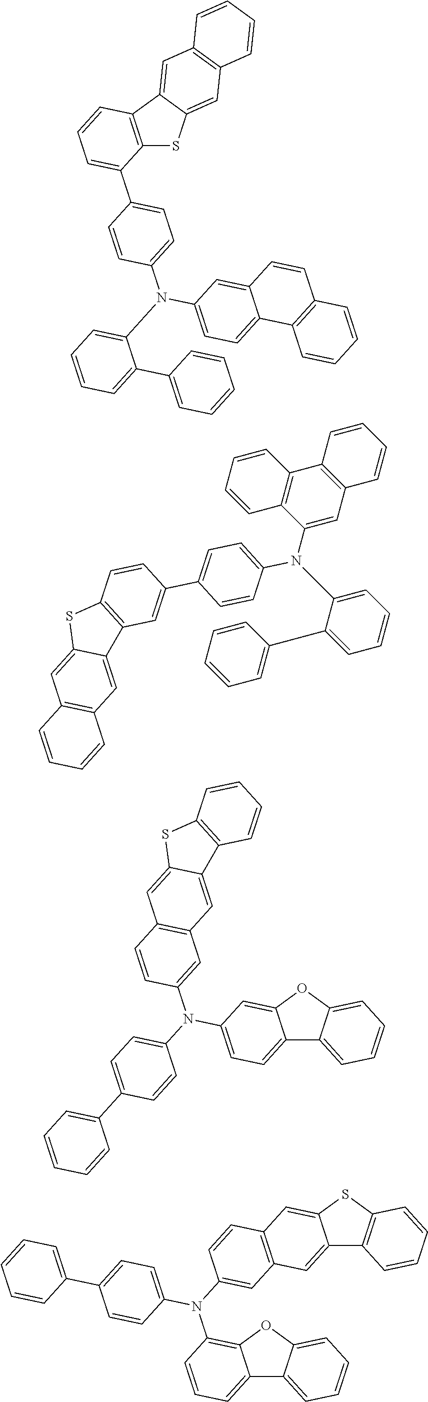 [SMILES: c1ccc(-c2ccccc2N(c2ccc(-c3cccc4c3sc3cc5ccccc5cc34)cc2)c2ccc3c(ccc4ccccc43)c2)cc1 ; c1ccc(-c2ccccc2N(c2ccc(-c3ccc4sc5cc6ccccc6cc5c4c3)cc2)c2cc3ccccc3c3ccccc23)cc1 ; c1ccc(-c2ccc(N(c3ccc4cc5sc6ccccc6c5cc4c3)c3ccc4c(c3)oc3ccccc34)cc2)cc1 ; c1ccc(-c2ccc(N(c3ccc4cc5sc6ccccc6c5cc4c3)c3cccc4c3oc3ccccc34)cc2)cc1]

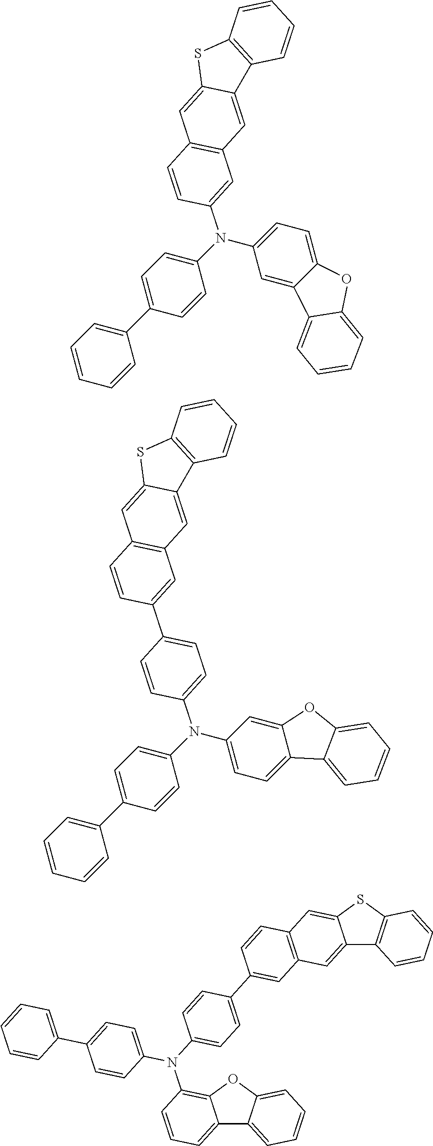 [SMILES: c1ccc(-c2ccc(N(c3ccc4cc5sc6ccccc6c5cc4c3)c3ccc4oc5ccccc5c4c3)cc2)cc1 ; c1ccc(-c2ccc(N(c3ccc(-c4ccc5cc6sc7ccccc7c6cc5c4)cc3)c3ccc4c(c3)oc3ccccc34)cc2)cc1 ; c1ccc(-c2ccc(N(c3ccc(-c4ccc5cc6sc7ccccc7c6cc5c4)cc3)c3cccc4c3oc3ccccc34)cc2)cc1]

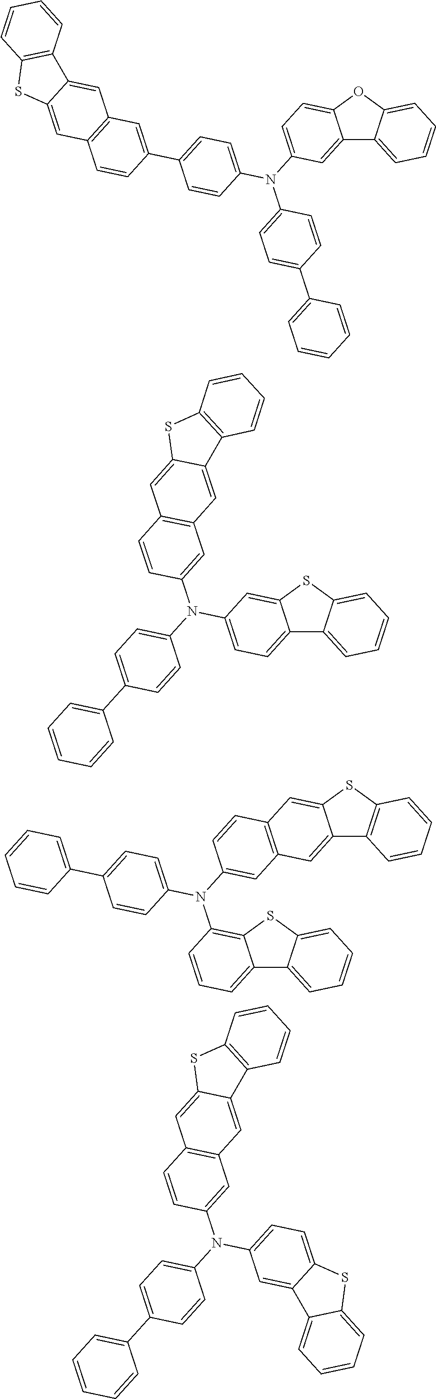

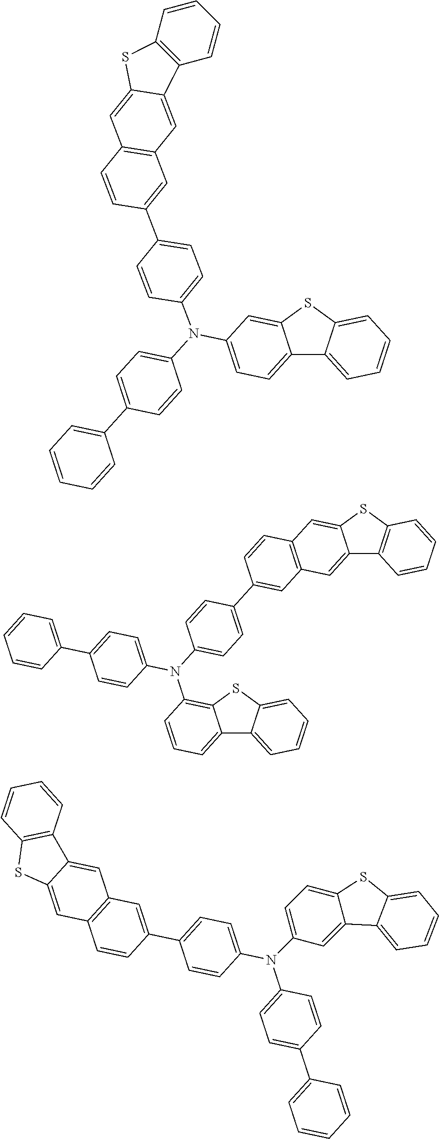 [SMILES: c1ccc(-c2ccc(N(c3ccc(-c4ccc5cc6sc7ccccc7c6cc5c4)cc3)c3ccc4c(c3)sc3ccccc34)cc2)cc1 ; c1ccc(-c2ccc(N(c3ccc(-c4ccc5cc6sc7ccccc7c6cc5c4)cc3)c3cccc4c3sc3ccccc34)cc2)cc1 ; c1ccc(-c2ccc(N(c3ccc(-c4ccc5cc6sc7ccccc7c6cc5c4)cc3)c3ccc4sc5ccccc5c4c3)cc2)cc1]

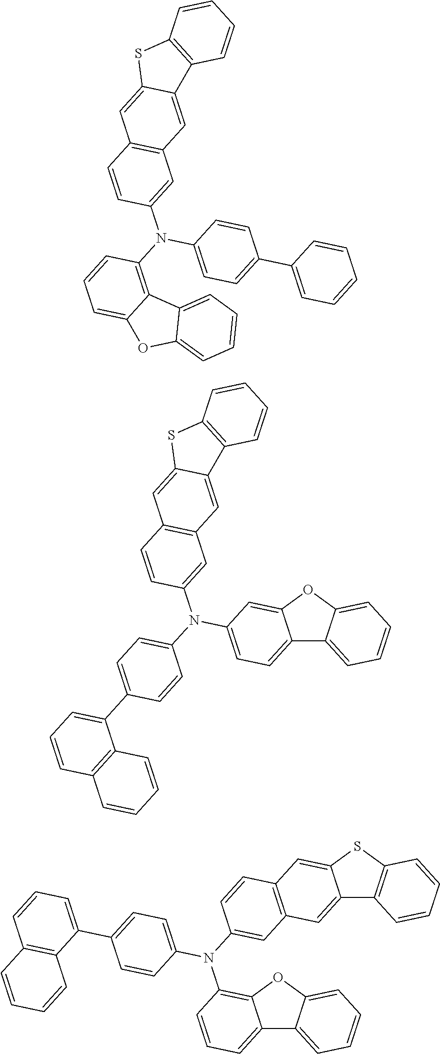

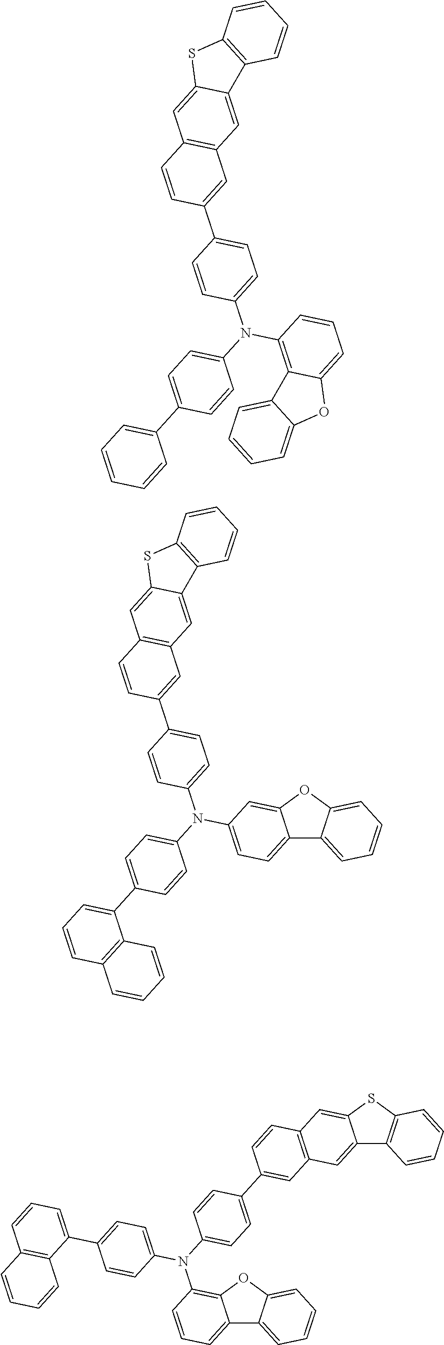 [SMILES: c1ccc(-c2ccc(N(c3ccc(-c4ccc5cc6sc7ccccc7c6cc5c4)cc3)c3cccc4oc5ccccc5c34)cc2)cc1 ; c1ccc2c(-c3ccc(N(c4ccc(-c5ccc6cc7sc8ccccc8c7cc6c5)cc4)c4ccc5c(c4)oc4ccccc45)cc3)cccc2c1 ; c1ccc2c(-c3ccc(N(c4ccc(-c5ccc6cc7sc8ccccc8c7cc6c5)cc4)c4cccc5c4oc4ccccc45)cc3)cccc2c1]

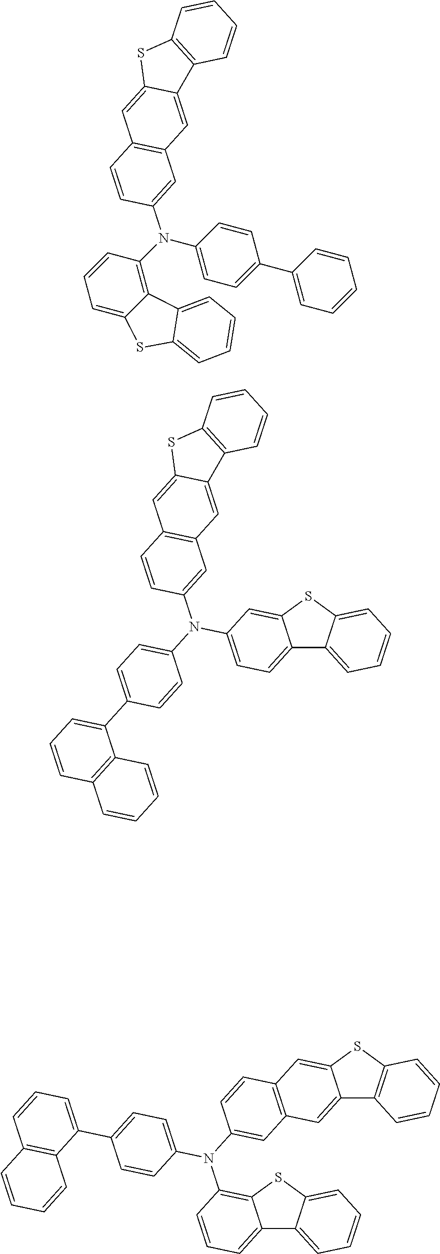

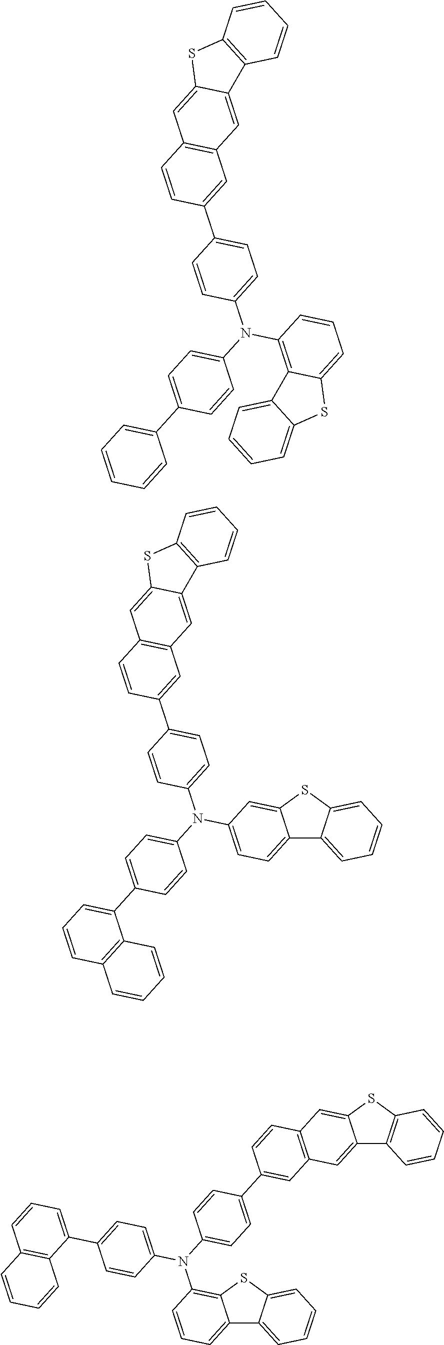 [SMILES: c1ccc(-c2ccc(N(c3ccc(-c4ccc5cc6sc7ccccc7c6cc5c4)cc3)c3cccc4sc5ccccc5c34)cc2)cc1 ; c1ccc2c(-c3ccc(N(c4ccc(-c5ccc6cc7sc8ccccc8c7cc6c5)cc4)c4ccc5c(c4)sc4ccccc45)cc3)cccc2c1 ; c1ccc2c(-c3ccc(N(c4ccc(-c5ccc6cc7sc8ccccc8c7cc6c5)cc4)c4cccc5c4sc4ccccc45)cc3)cccc2c1]

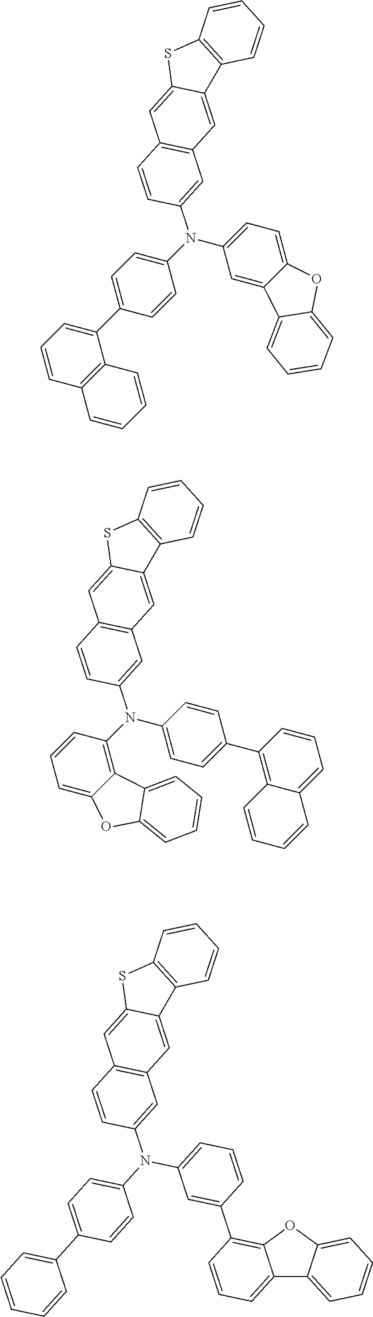 [SMILES: c1ccc2c(-c3ccc(N(c4ccc5cc6sc7ccccc7c6cc5c4)c4ccc5oc6ccccc6c5c4)cc3)cccc2c1 ; c1ccc2c(-c3ccc(N(c4ccc5cc6sc7ccccc7c6cc5c4)c4cccc5oc6ccccc6c45)cc3)cccc2c1 ; c1ccc(-c2ccc(N(c3cccc(-c4cccc5c4oc4ccccc45)c3)c3ccc4cc5sc6ccccc6c5cc4c3)cc2)cc1]

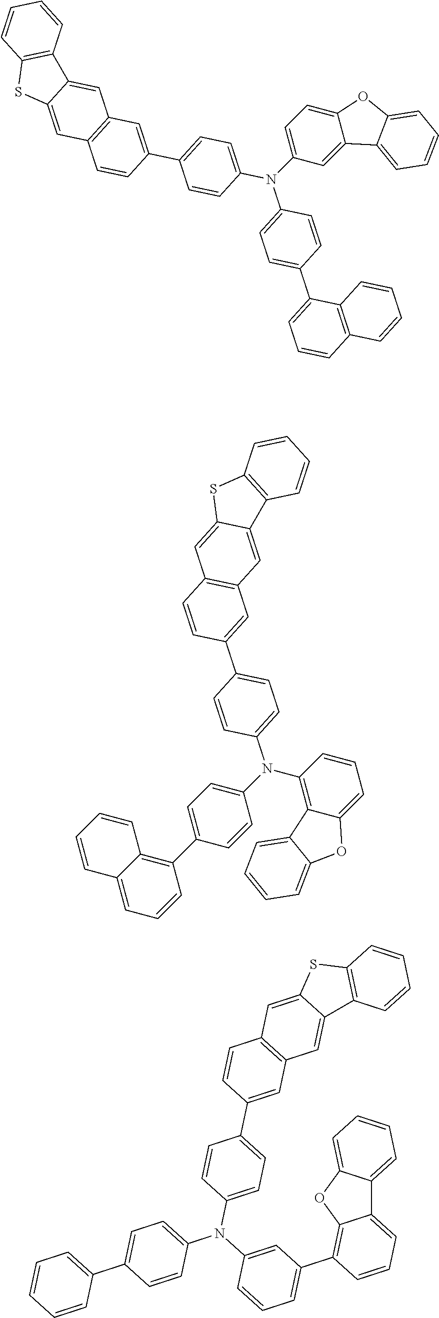 [SMILES: c1ccc2c(-c3ccc(N(c4ccc(-c5ccc6cc7sc8ccccc8c7cc6c5)cc4)c4ccc5oc6ccccc6c5c4)cc3)cccc2c1 ; c1ccc2c(-c3ccc(N(c4ccc(-c5ccc6cc7sc8ccccc8c7cc6c5)cc4)c4cccc5oc6ccccc6c45)cc3)cccc2c1 ; c1ccc(-c2ccc(N(c3ccc(-c4ccc5cc6sc7ccccc7c6cc5c4)cc3)c3cccc(-c4cccc5c4oc4ccccc45)c3)cc2)cc1]

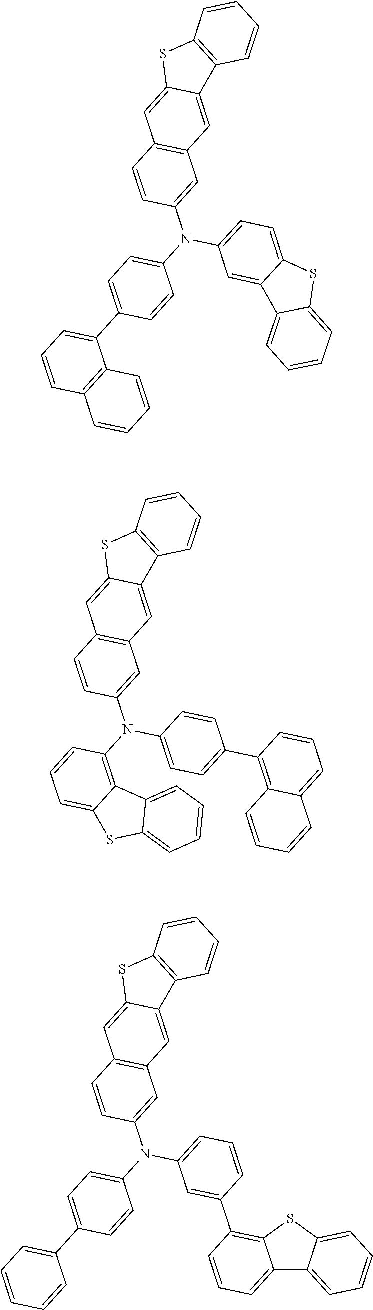 [SMILES: c1ccc2c(-c3ccc(N(c4ccc5cc6sc7ccccc7c6cc5c4)c4ccc5sc6ccccc6c5c4)cc3)cccc2c1 ; c1ccc2c(-c3ccc(N(c4ccc5cc6sc7ccccc7c6cc5c4)c4cccc5sc6ccccc6c45)cc3)cccc2c1 ; c1ccc(-c2ccc(N(c3cccc(-c4cccc5c4sc4ccccc45)c3)c3ccc4cc5sc6ccccc6c5cc4c3)cc2)cc1]

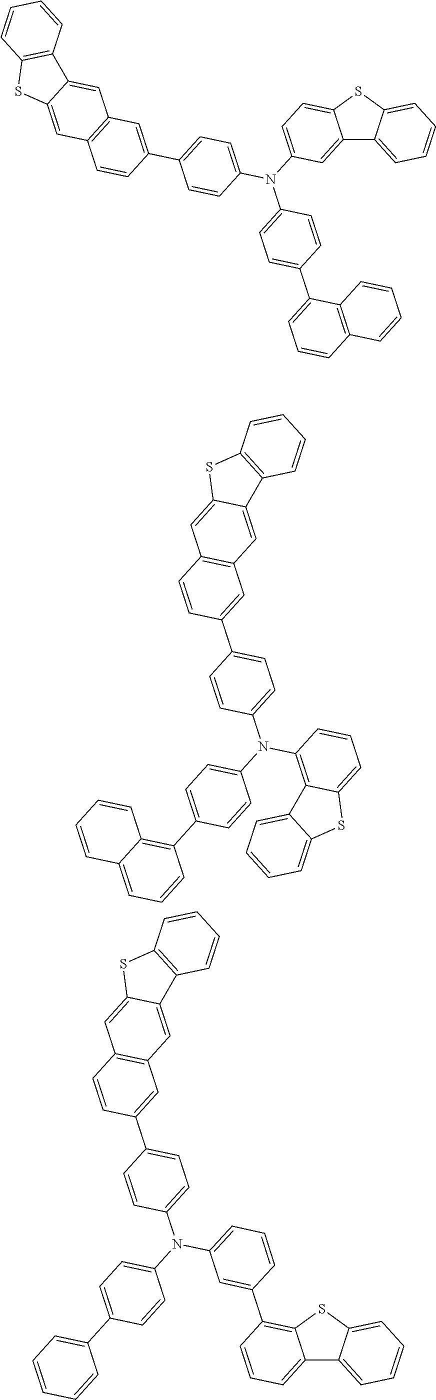

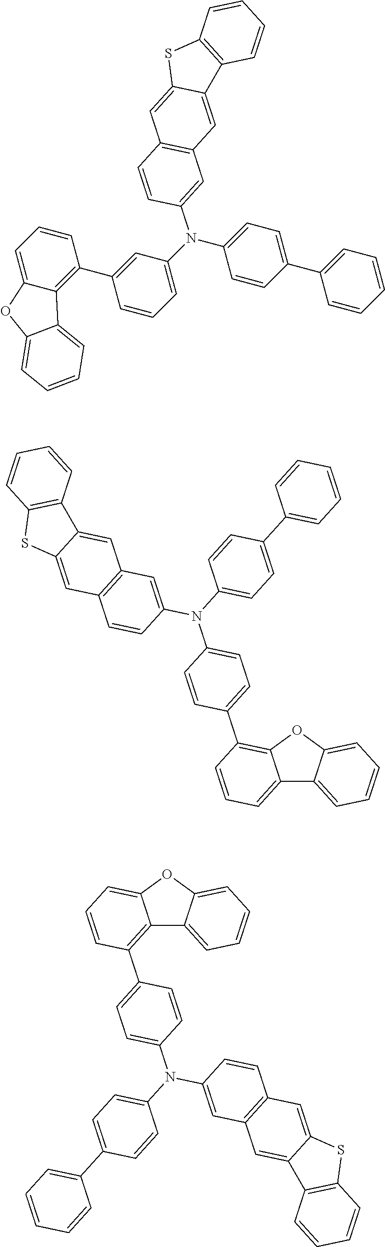 [SMILES: c1ccc(-c2ccc(N(c3cccc(-c4cccc5oc6ccccc6c45)c3)c3ccc4cc5sc6ccccc6c5cc4c3)cc2)cc1 ; c1ccc(-c2ccc(N(c3ccc(-c4cccc5c4oc4ccccc45)cc3)c3ccc4cc5sc6ccccc6c5cc4c3)cc2)cc1 ; c1ccc(-c2ccc(N(c3ccc(-c4cccc5oc6ccccc6c45)cc3)c3ccc4cc5sc6ccccc6c5cc4c3)cc2)cc1]

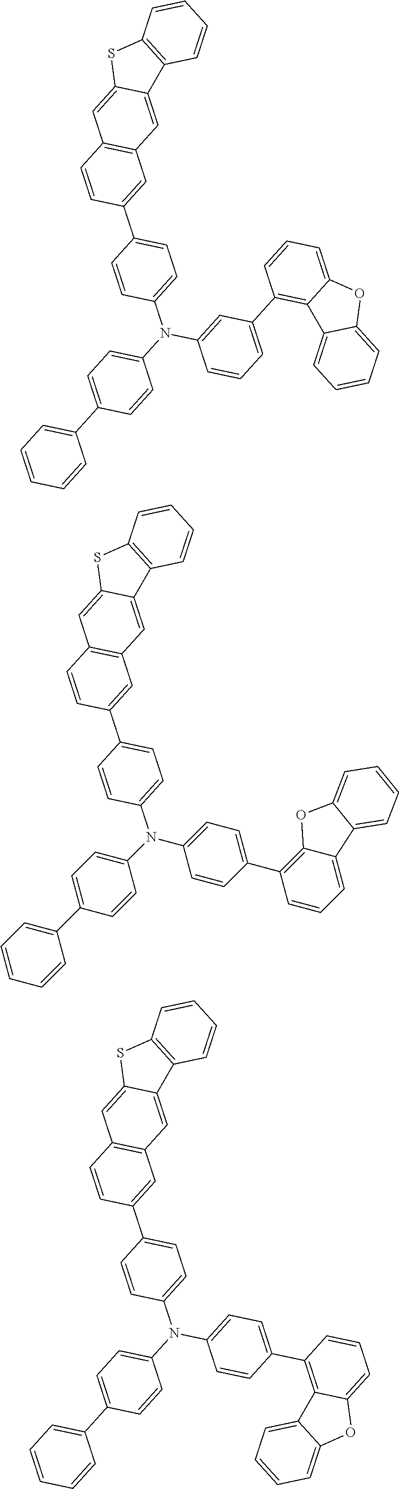 [SMILES: c1ccc(-c2ccc(N(c3ccc(-c4ccc5cc6sc7ccccc7c6cc5c4)cc3)c3cccc(-c4cccc5oc6ccccc6c45)c3)cc2)cc1 ; c1ccc(-c2ccc(N(c3ccc(-c4ccc5cc6sc7ccccc7c6cc5c4)cc3)c3ccc(-c4cccc5c4oc4ccccc45)cc3)cc2)cc1 ; c1ccc(-c2ccc(N(c3ccc(-c4ccc5cc6sc7ccccc7c6cc5c4)cc3)c3ccc(-c4cccc5oc6ccccc6c45)cc3)cc2)cc1]

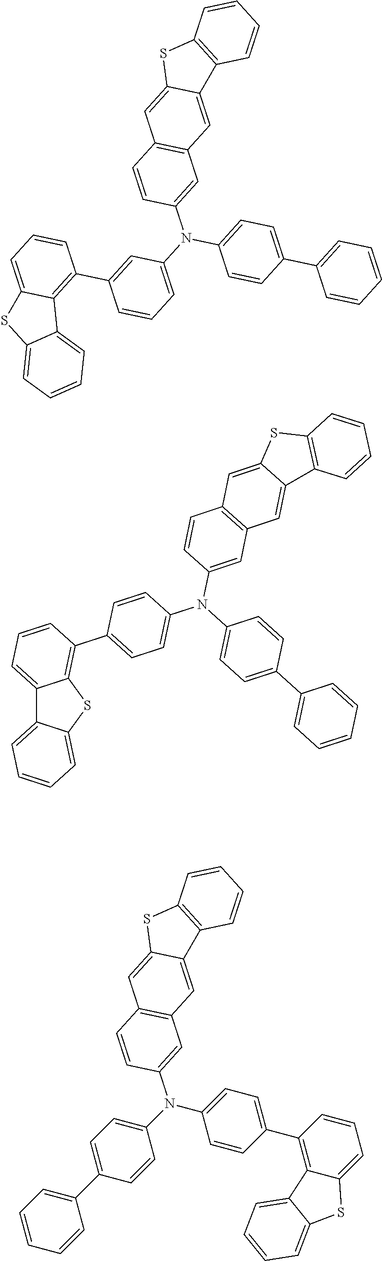 [SMILES: c1ccc(-c2ccc(N(c3cccc(-c4cccc5sc6ccccc6c45)c3)c3ccc4cc5sc6ccccc6c5cc4c3)cc2)cc1 ; c1ccc(-c2ccc(N(c3ccc(-c4cccc5c4sc4ccccc45)cc3)c3ccc4cc5sc6ccccc6c5cc4c3)cc2)cc1 ; c1ccc(-c2ccc(N(c3ccc(-c4cccc5sc6ccccc6c45)cc3)c3ccc4cc5sc6ccccc6c5cc4c3)cc2)cc1]

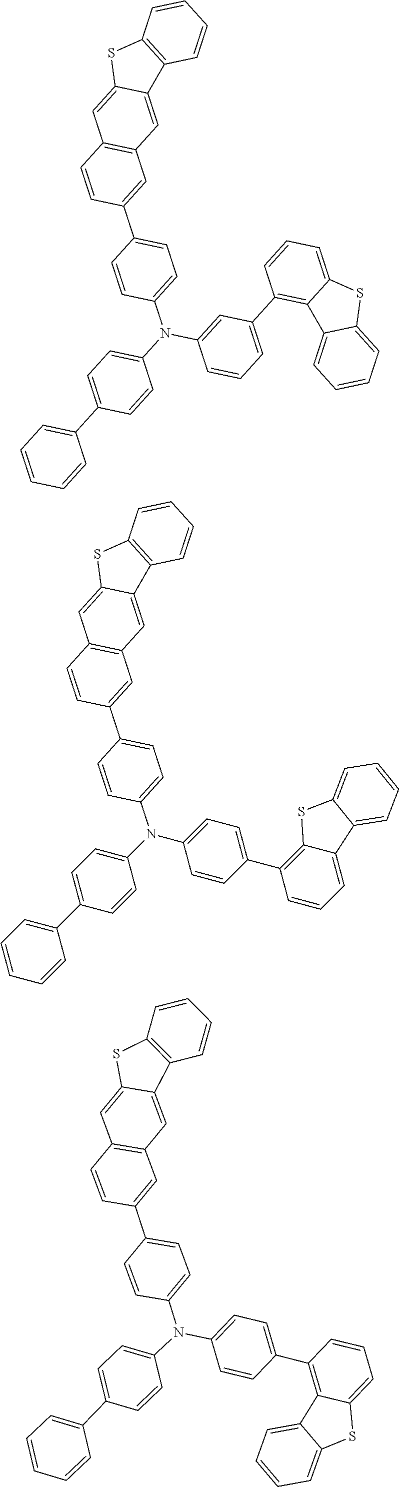 [SMILES: c1ccc(-c2ccc(N(c3ccc(-c4ccc5cc6sc7ccccc7c6cc5c4)cc3)c3cccc(-c4cccc5sc6ccccc6c45)c3)cc2)cc1 ; c1ccc(-c2ccc(N(c3ccc(-c4ccc5cc6sc7ccccc7c6cc5c4)cc3)c3ccc(-c4cccc5c4sc4ccccc45)cc3)cc2)cc1 ; c1ccc(-c2ccc(N(c3ccc(-c4ccc5cc6sc7ccccc7c6cc5c4)cc3)c3ccc(-c4cccc5sc6ccccc6c45)cc3)cc2)cc1]

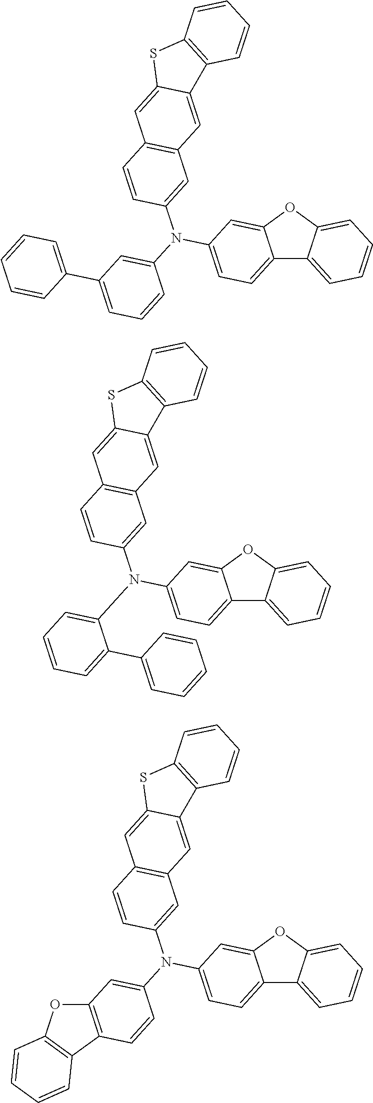

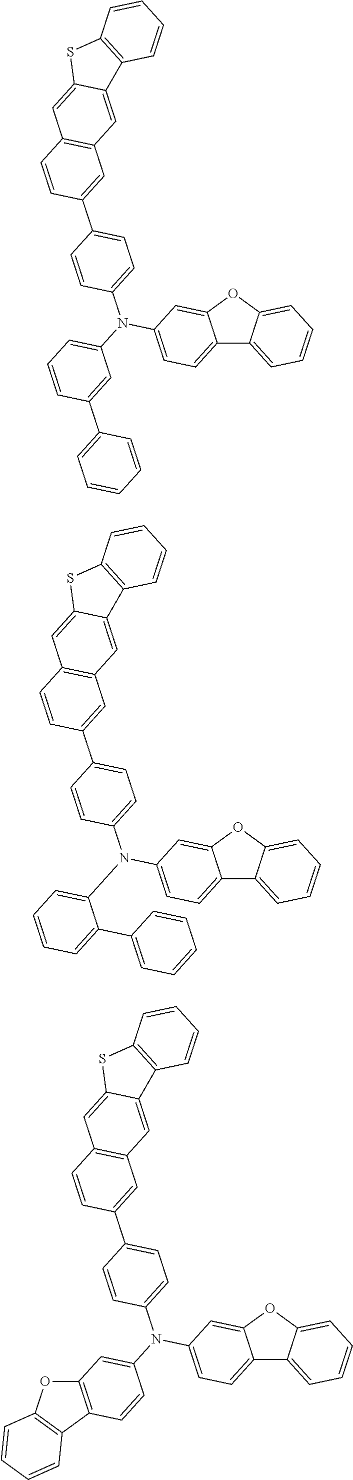

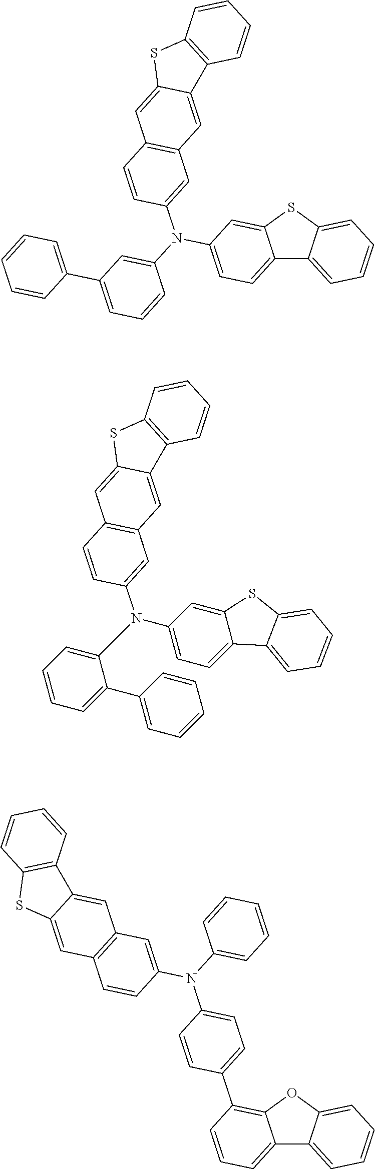 [SMILES: c1ccc(-c2cccc(N(c3ccc4cc5sc6ccccc6c5cc4c3)c3ccc4c(c3)sc3ccccc34)c2)cc1 ; c1ccc(-c2ccccc2N(c2ccc3cc4sc5ccccc5c4cc3c2)c2ccc3c(c2)sc2ccccc23)cc1 ; c1ccc(N(c2ccc(-c3cccc4c3oc3ccccc34)cc2)c2ccc3cc4sc5ccccc5c4cc3c2)cc1]

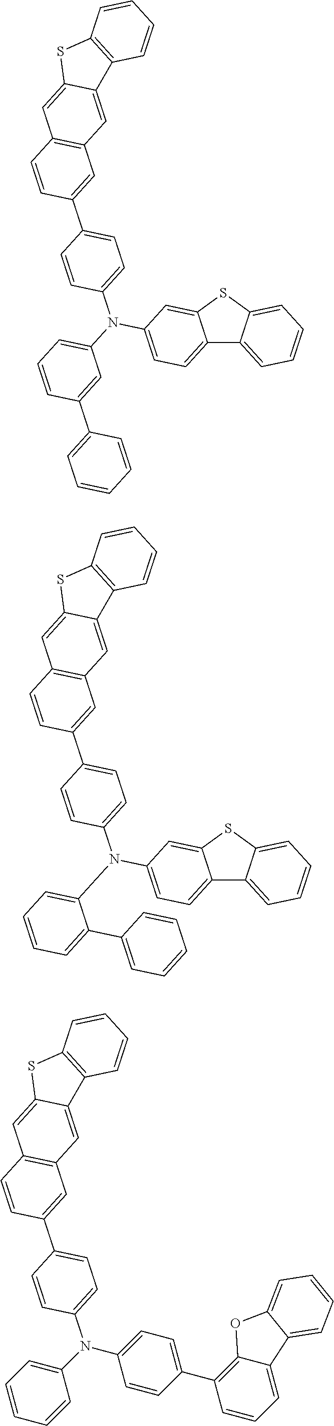

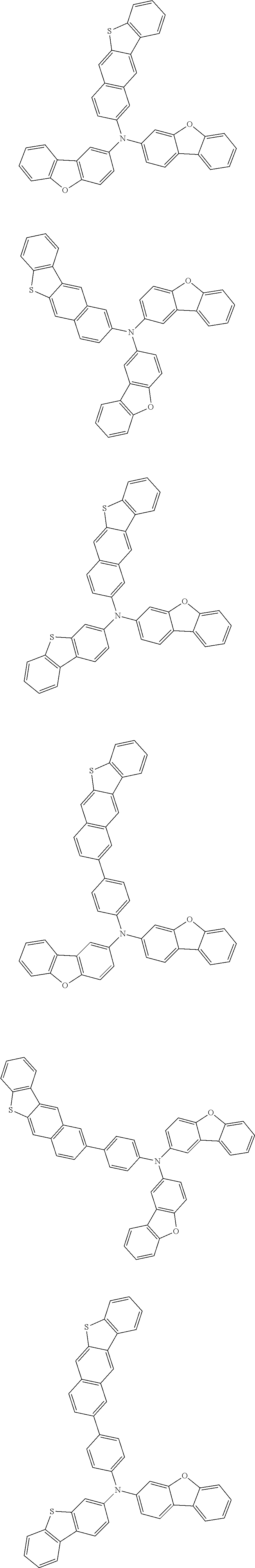 [SMILES: c1ccc2c(c1)oc1cc(N(c3ccc4cc5sc6ccccc6c5cc4c3)c3ccc4oc5ccccc5c4c3)ccc12 ; c1ccc2c(c1)oc1ccc(N(c3ccc4cc5sc6ccccc6c5cc4c3)c3ccc4oc5ccccc5c4c3)cc12 ; c1ccc2c(c1)oc1cc(N(c3ccc4cc5sc6ccccc6c5cc4c3)c3ccc4c(c3)sc3ccccc34)ccc12 ; c1ccc2c(c1)oc1cc(N(c3ccc(-c4ccc5cc6sc7ccccc7c6cc5c4)cc3)c3ccc4oc5ccccc5c4c3)ccc12 ; c1ccc2c(c1)oc1ccc(N(c3ccc(-c4ccc5cc6sc7ccccc7c6cc5c4)cc3)c3ccc4oc5ccccc5c4c3)cc12 ; c1ccc2c(c1)oc1cc(N(c3ccc(-c4ccc5cc6sc7ccccc7c6cc5c4)cc3)c3ccc4c(c3)sc3ccccc34)ccc12]

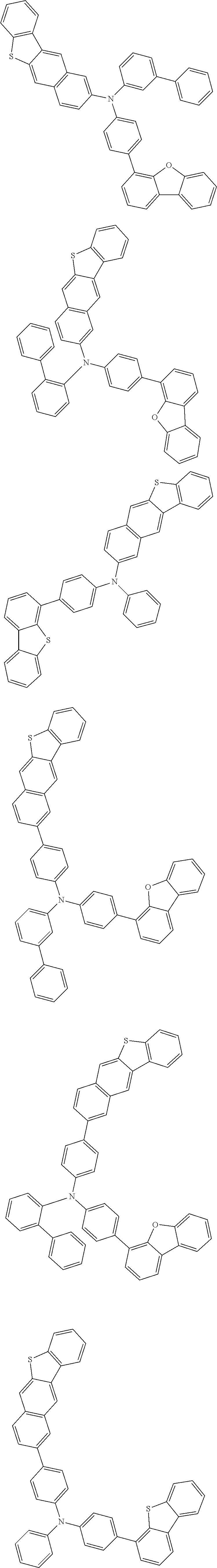

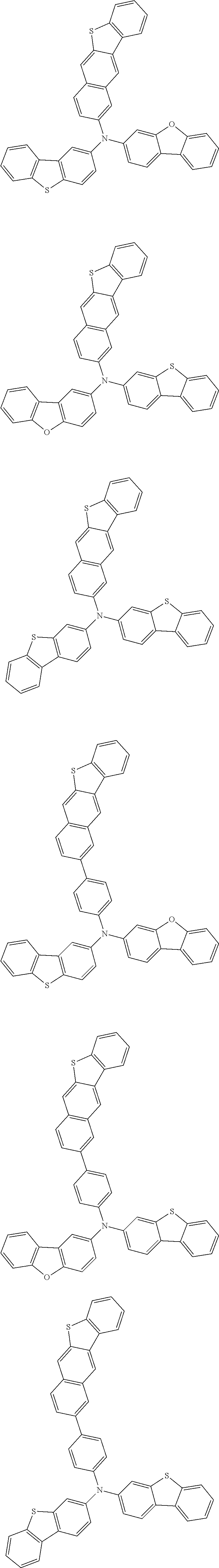 [SMILES: c1ccc2c(c1)oc1cc(N(c3ccc4cc5sc6ccccc6c5cc4c3)c3ccc4sc5ccccc5c4c3)ccc12 ; c1ccc2c(c1)oc1ccc(N(c3ccc4cc5sc6ccccc6c5cc4c3)c3ccc4c(c3)sc3ccccc34)cc12 ; c1ccc2c(c1)sc1cc(N(c3ccc4cc5sc6ccccc6c5cc4c3)c3ccc4c(c3)sc3ccccc34)ccc12 ; c1ccc2c(c1)oc1cc(N(c3ccc(-c4ccc5cc6sc7ccccc7c6cc5c4)cc3)c3ccc4sc5ccccc5c4c3)ccc12 ; c1ccc2c(c1)oc1ccc(N(c3ccc(-c4ccc5cc6sc7ccccc7c6cc5c4)cc3)c3ccc4c(c3)sc3ccccc34)cc12 ; c1ccc2c(c1)sc1cc(N(c3ccc(-c4ccc5cc6sc7ccccc7c6cc5c4)cc3)c3ccc4c(c3)sc3ccccc34)ccc12]

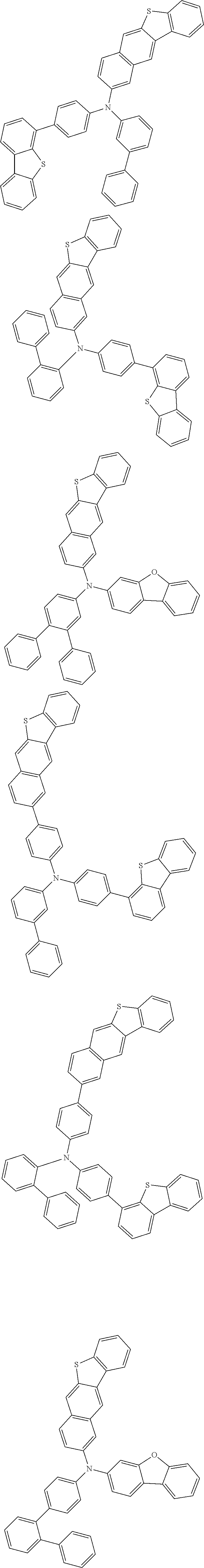

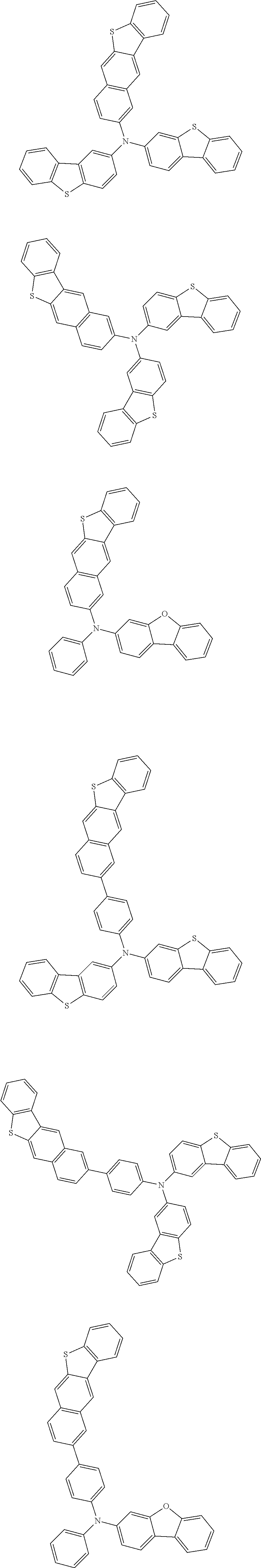 [SMILES: c1ccc2c(c1)sc1cc(N(c3ccc4cc5sc6ccccc6c5cc4c3)c3ccc4sc5ccccc5c4c3)ccc12 ; c1ccc2c(c1)sc1ccc(N(c3ccc4cc5sc6ccccc6c5cc4c3)c3ccc4sc5ccccc5c4c3)cc12 ; c1ccc(N(c2ccc3cc4sc5ccccc5c4cc3c2)c2ccc3c(c2)oc2ccccc23)cc1 ; c1ccc2c(c1)sc1cc(N(c3ccc(-c4ccc5cc6sc7ccccc7c6cc5c4)cc3)c3ccc4sc5ccccc5c4c3)ccc12 ; c1ccc2c(c1)sc1ccc(N(c3ccc(-c4ccc5cc6sc7ccccc7c6cc5c4)cc3)c3ccc4sc5ccccc5c4c3)cc12 ; c1ccc(N(c2ccc(-c3ccc4cc5sc6ccccc6c5cc4c3)cc2)c2ccc3c(c2)oc2ccccc23)cc1]

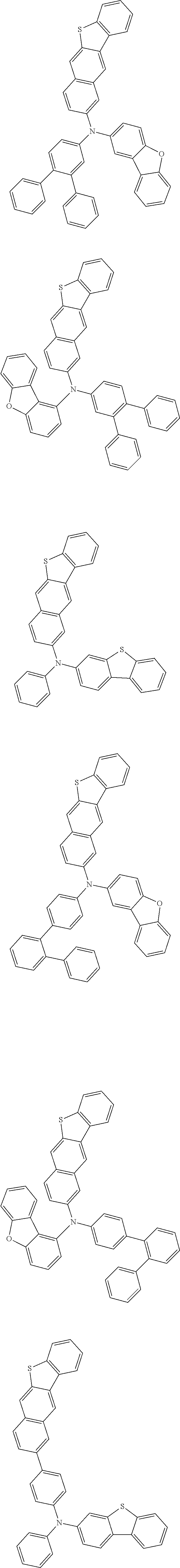 [SMILES: c1ccc(-c2ccc(N(c3ccc4cc5sc6ccccc6c5cc4c3)c3ccc4oc5ccccc5c4c3)cc2-c2ccccc2)cc1 ; c1ccc(-c2ccc(N(c3ccc4cc5sc6ccccc6c5cc4c3)c3cccc4oc5ccccc5c34)cc2-c2ccccc2)cc1 ; c1ccc(N(c2ccc3cc4sc5ccccc5c4cc3c2)c2ccc3c(c2)sc2ccccc23)cc1 ; c1ccc(-c2ccccc2-c2ccc(N(c3ccc4cc5sc6ccccc6c5cc4c3)c3ccc4oc5ccccc5c4c3)cc2)cc1 ; c1ccc(-c2ccccc2-c2ccc(N(c3ccc4cc5sc6ccccc6c5cc4c3)c3cccc4oc5ccccc5c34)cc2)cc1 ; c1ccc(N(c2ccc(-c3ccc4cc5sc6ccccc6c5cc4c3)cc2)c2ccc3c(c2)sc2ccccc23)cc1]

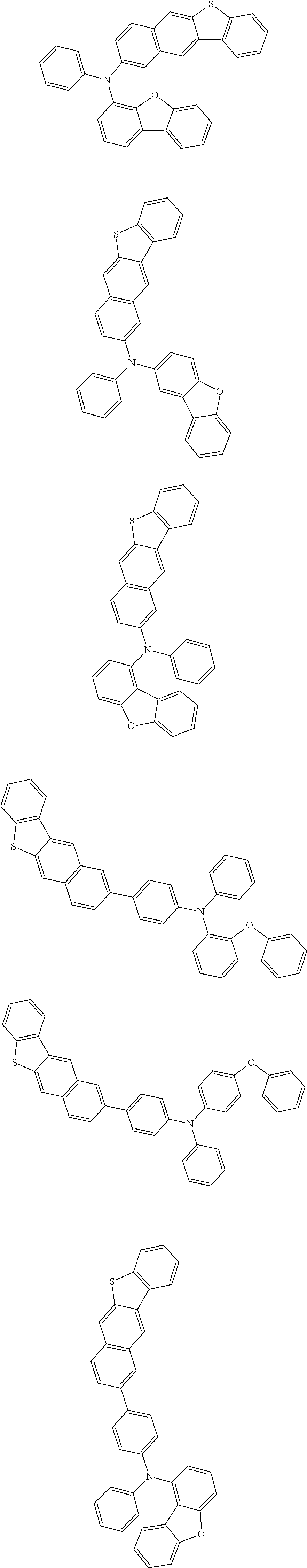

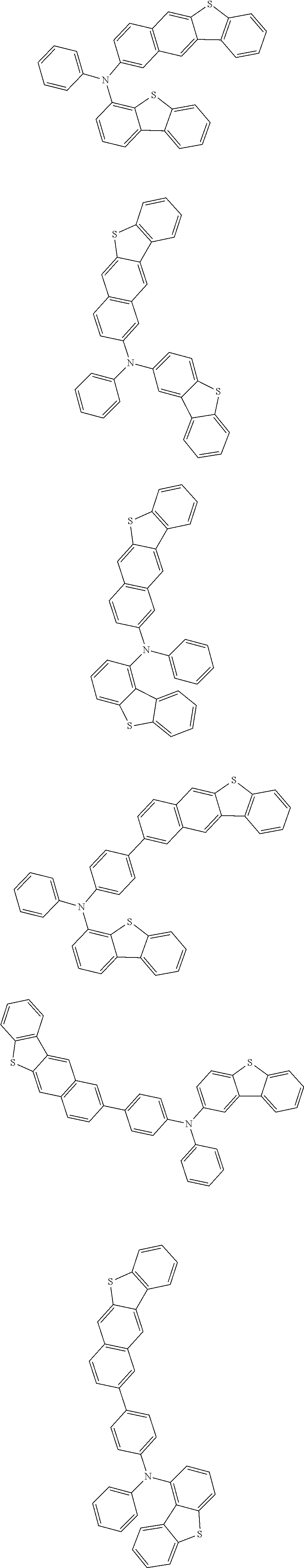

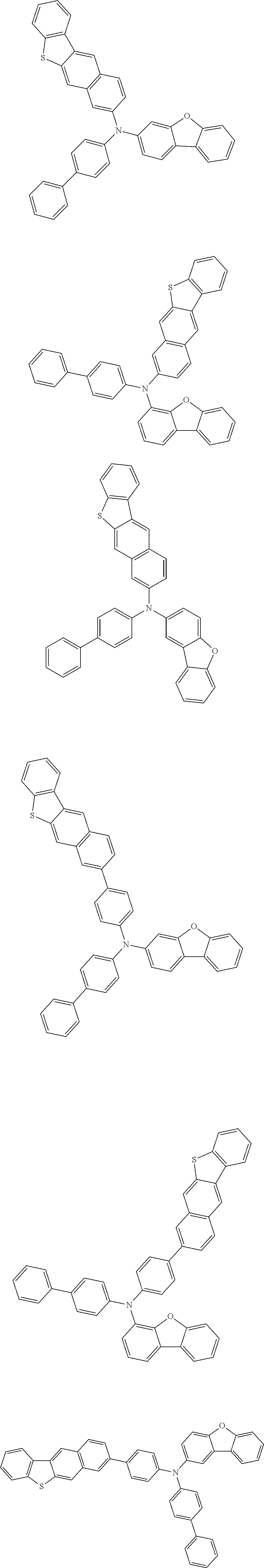 [SMILES: c1ccc(-c2ccc(N(c3ccc4cc5c(cc4c3)sc3ccccc35)c3ccc4c(c3)oc3ccccc34)cc2)cc1 ; c1ccc(-c2ccc(N(c3ccc4cc5c(cc4c3)sc3ccccc35)c3cccc4c3oc3ccccc34)cc2)cc1 ; c1ccc(-c2ccc(N(c3ccc4cc5c(cc4c3)sc3ccccc35)c3ccc4oc5ccccc5c4c3)cc2)cc1 ; c1ccc(-c2ccc(N(c3ccc(-c4ccc5cc6c(cc5c4)sc4ccccc46)cc3)c3ccc4c(c3)oc3ccccc34)cc2)cc1 ; c1ccc(-c2ccc(N(c3ccc(-c4ccc5cc6c(cc5c4)sc4ccccc46)cc3)c3cccc4c3oc3ccccc34)cc2)cc1 ; c1ccc(-c2ccc(N(c3ccc(-c4ccc5cc6c(cc5c4)sc4ccccc46)cc3)c3ccc4oc5ccccc5c4c3)cc2)cc1]

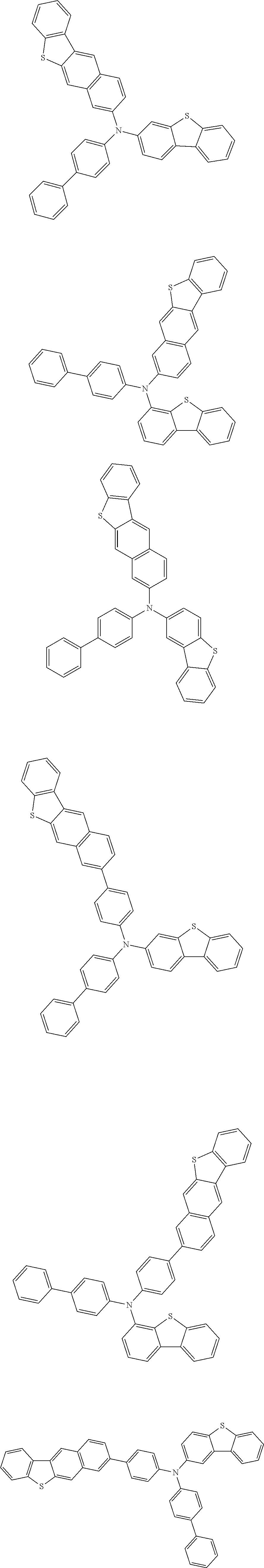 [SMILES: c1ccc(-c2ccc(N(c3ccc4cc5c(cc4c3)sc3ccccc35)c3ccc4c(c3)sc3ccccc34)cc2)cc1 ; c1ccc(-c2ccc(N(c3ccc4cc5c(cc4c3)sc3ccccc35)c3cccc4c3sc3ccccc34)cc2)cc1 ; c1ccc(-c2ccc(N(c3ccc4cc5c(cc4c3)sc3ccccc35)c3ccc4sc5ccccc5c4c3)cc2)cc1 ; c1ccc(-c2ccc(N(c3ccc(-c4ccc5cc6c(cc5c4)sc4ccccc46)cc3)c3ccc4c(c3)sc3ccccc34)cc2)cc1 ; c1ccc(-c2ccc(N(c3ccc(-c4ccc5cc6c(cc5c4)sc4ccccc46)cc3)c3cccc4c3sc3ccccc34)cc2)cc1 ; c1ccc(-c2ccc(N(c3ccc(-c4ccc5cc6c(cc5c4)sc4ccccc46)cc3)c3ccc4sc5ccccc5c4c3)cc2)cc1]

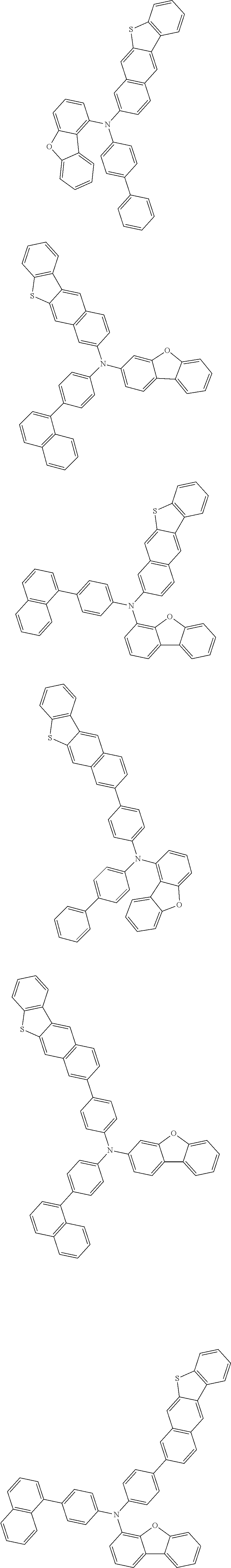 [SMILES: c1ccc(-c2ccc(N(c3ccc4cc5c(cc4c3)sc3ccccc35)c3cccc4oc5ccccc5c34)cc2)cc1 ; c1ccc2c(-c3ccc(N(c4ccc5cc6c(cc5c4)sc4ccccc46)c4ccc5c(c4)oc4ccccc45)cc3)cccc2c1 ; c1ccc2c(-c3ccc(N(c4ccc5cc6c(cc5c4)sc4ccccc46)c4cccc5c4oc4ccccc45)cc3)cccc2c1 ; c1ccc(-c2ccc(N(c3ccc(-c4ccc5cc6c(cc5c4)sc4ccccc46)cc3)c3cccc4oc5ccccc5c34)cc2)cc1 ; c1ccc2c(-c3ccc(N(c4ccc(-c5ccc6cc7c(cc6c5)sc5ccccc57)cc4)c4ccc5c(c4)oc4ccccc45)cc3)cccc2c1 ; c1ccc2c(-c3ccc(N(c4ccc(-c5ccc6cc7c(cc6c5)sc5ccccc57)cc4)c4cccc5c4oc4ccccc45)cc3)cccc2c1]

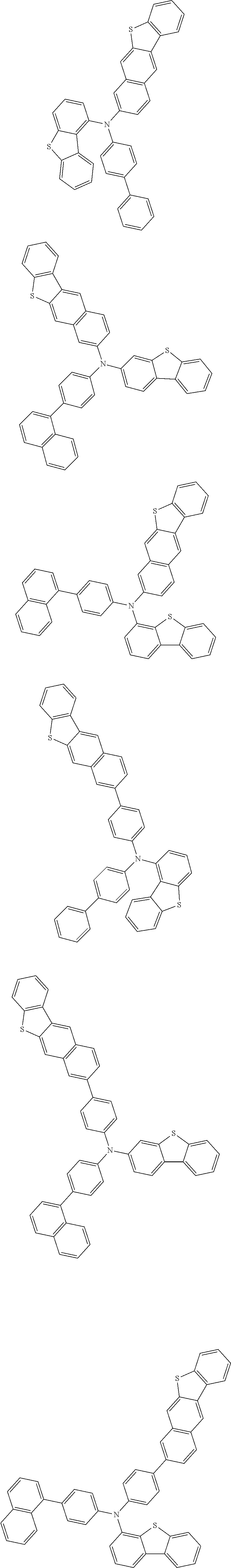 [SMILES: c1ccc(-c2ccc(N(c3ccc4cc5c(cc4c3)sc3ccccc35)c3cccc4sc5ccccc5c34)cc2)cc1 ; c1ccc2c(-c3ccc(N(c4ccc5cc6c(cc5c4)sc4ccccc46)c4ccc5c(c4)sc4ccccc45)cc3)cccc2c1 ; c1ccc2c(-c3ccc(N(c4ccc5cc6c(cc5c4)sc4ccccc46)c4cccc5c4sc4ccccc45)cc3)cccc2c1 ; c1ccc(-c2ccc(N(c3ccc(-c4ccc5cc6c(cc5c4)sc4ccccc46)cc3)c3cccc4sc5ccccc5c34)cc2)cc1 ; c1ccc2c(-c3ccc(N(c4ccc(-c5ccc6cc7c(cc6c5)sc5ccccc57)cc4)c4ccc5c(c4)sc4ccccc45)cc3)cccc2c1 ; c1ccc2c(-c3ccc(N(c4ccc(-c5ccc6cc7c(cc6c5)sc5ccccc57)cc4)c4cccc5c4sc4ccccc45)cc3)cccc2c1]

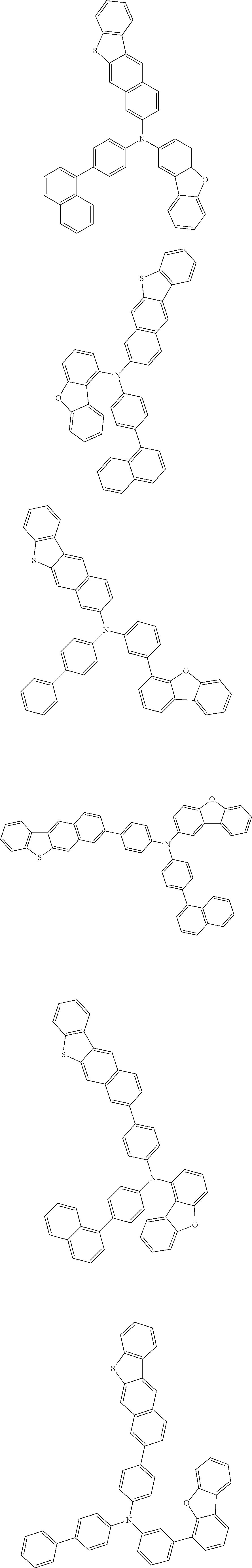

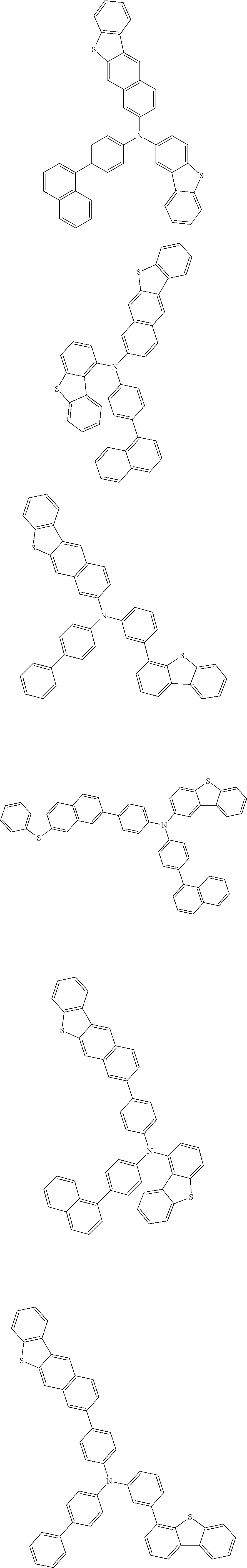

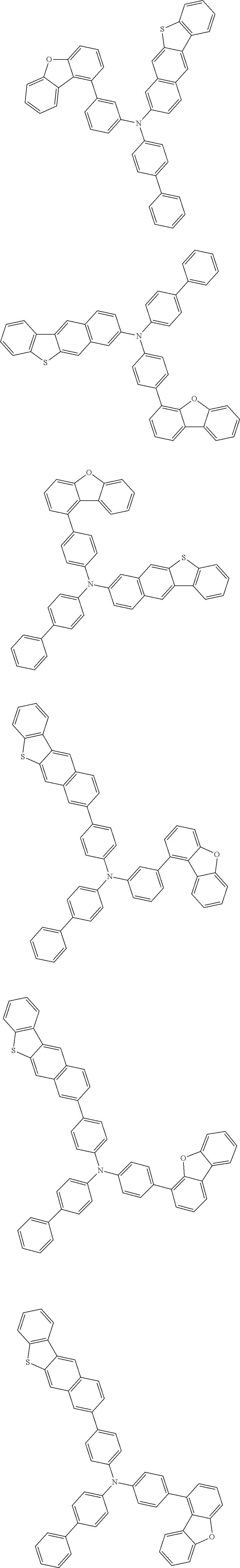

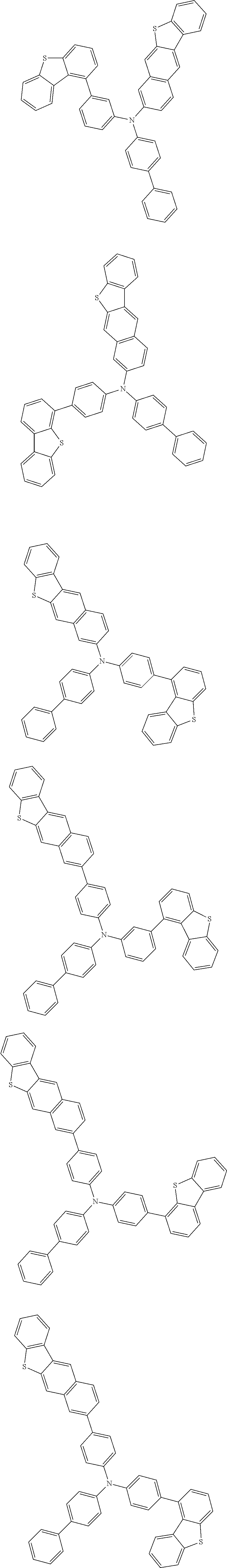

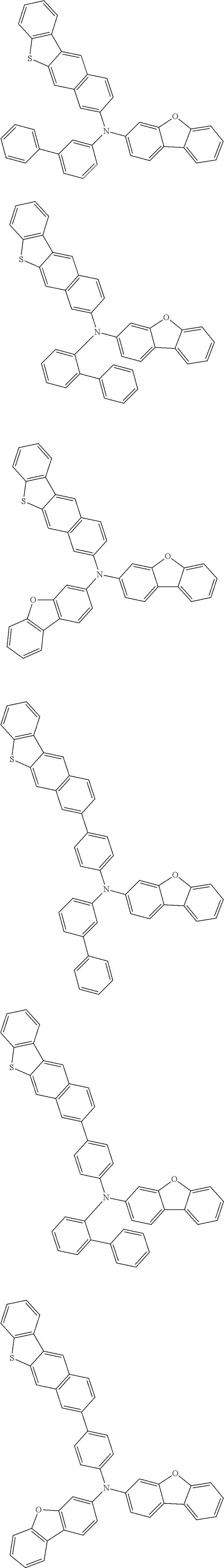 [SMILES: c1ccc(-c2cccc(N(c3ccc4cc5c(cc4c3)sc3ccccc35)c3ccc4c(c3)oc3ccccc34)c2)cc1 ; c1ccc(-c2ccccc2N(c2ccc3cc4c(cc3c2)sc2ccccc24)c2ccc3c(c2)oc2ccccc23)cc1 ; c1ccc2c(c1)oc1cc(N(c3ccc4cc5c(cc4c3)sc3ccccc35)c3ccc4c(c3)oc3ccccc34)ccc12 ; c1ccc(-c2cccc(N(c3ccc(-c4ccc5cc6c(cc5c4)sc4ccccc46)cc3)c3ccc4c(c3)oc3ccccc34)c2)cc1 ; c1ccc(-c2ccccc2N(c2ccc(-c3ccc4cc5c(cc4c3)sc3ccccc35)cc2)c2ccc3c(c2)oc2ccccc23)cc1 ; c1ccc2c(c1)oc1cc(N(c3ccc(-c4ccc5cc6c(cc5c4)sc4ccccc46)cc3)c3ccc4c(c3)oc3ccccc34)ccc12]

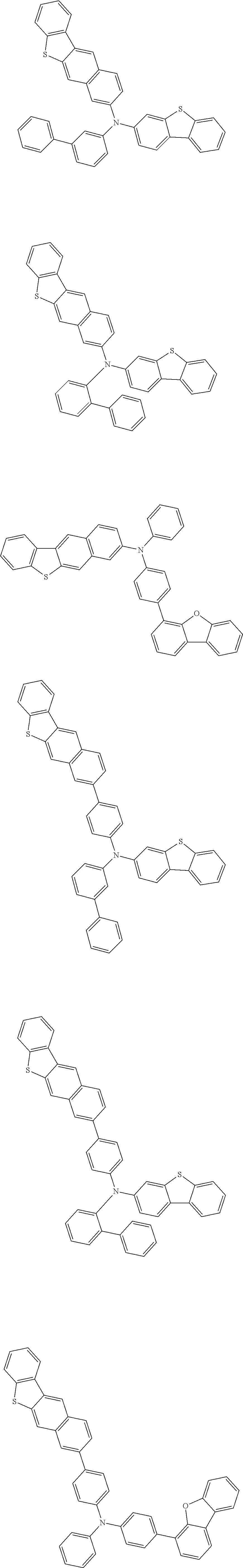 [SMILES: c1ccc(-c2cccc(N(c3ccc4cc5c(cc4c3)sc3ccccc35)c3ccc4c(c3)sc3ccccc34)c2)cc1 ; c1ccc(-c2ccccc2N(c2ccc3cc4c(cc3c2)sc2ccccc24)c2ccc3c(c2)sc2ccccc23)cc1 ; c1ccc(N(c2ccc(-c3cccc4c3oc3ccccc34)cc2)c2ccc3cc4c(cc3c2)sc2ccccc24)cc1 ; c1ccc(-c2cccc(N(c3ccc(-c4ccc5cc6c(cc5c4)sc4ccccc46)cc3)c3ccc4c(c3)sc3ccccc34)c2)cc1 ; c1ccc(-c2ccccc2N(c2ccc(-c3ccc4cc5c(cc4c3)sc3ccccc35)cc2)c2ccc3c(c2)sc2ccccc23)cc1 ; c1ccc(N(c2ccc(-c3ccc4cc5c(cc4c3)sc3ccccc35)cc2)c2ccc(-c3cccc4c3oc3ccccc34)cc2)cc1]

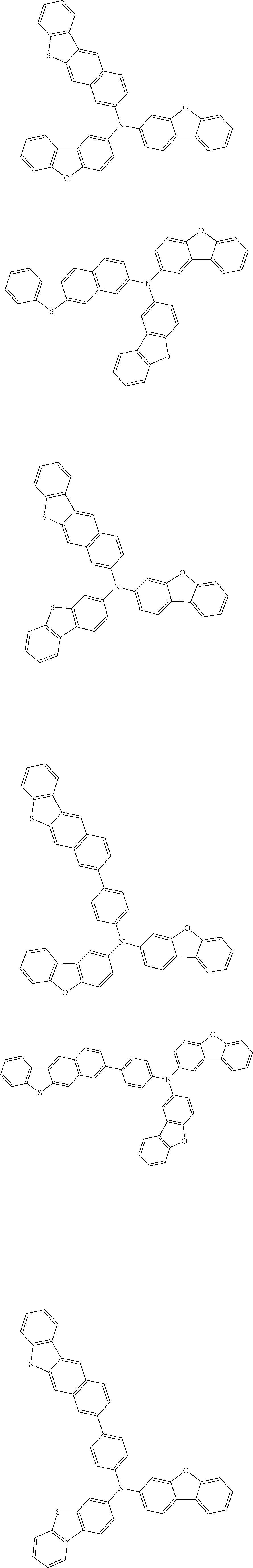 [SMILES: c1ccc2c(c1)oc1cc(N(c3ccc4cc5c(cc4c3)sc3ccccc35)c3ccc4oc5ccccc5c4c3)ccc12 ; c1ccc2c(c1)oc1ccc(N(c3ccc4cc5c(cc4c3)sc3ccccc35)c3ccc4oc5ccccc5c4c3)cc12 ; c1ccc2c(c1)oc1cc(N(c3ccc4cc5c(cc4c3)sc3ccccc35)c3ccc4c(c3)sc3ccccc34)ccc12 ; c1ccc2c(c1)oc1cc(N(c3ccc(-c4ccc5cc6c(cc5c4)sc4ccccc46)cc3)c3ccc4oc5ccccc5c4c3)ccc12 ; c1ccc2c(c1)oc1ccc(N(c3ccc(-c4ccc5cc6c(cc5c4)sc4ccccc46)cc3)c3ccc4oc5ccccc5c4c3)cc12 ; c1ccc2c(c1)oc1cc(N(c3ccc(-c4ccc5cc6c(cc5c4)sc4ccccc46)cc3)c3ccc4c(c3)sc3ccccc34)ccc12]

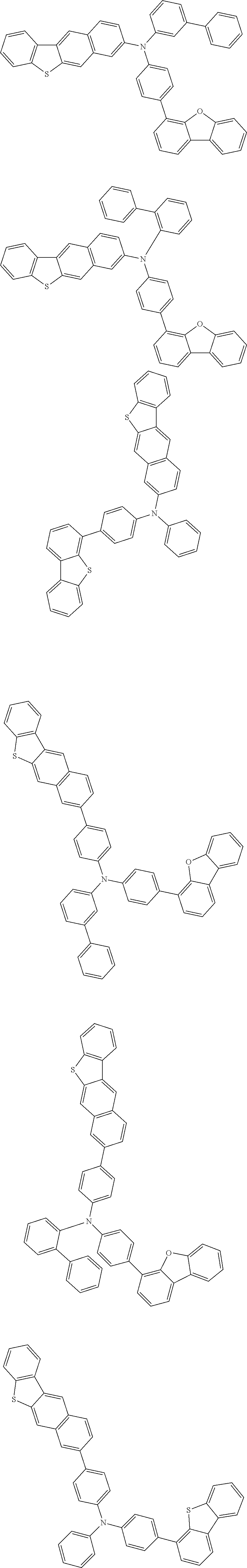

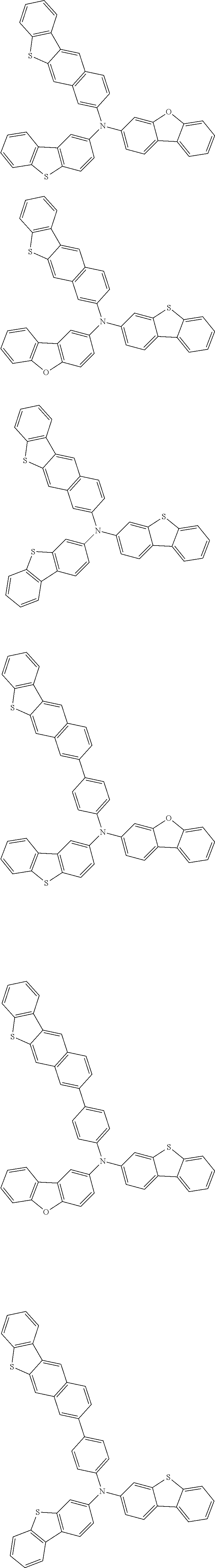

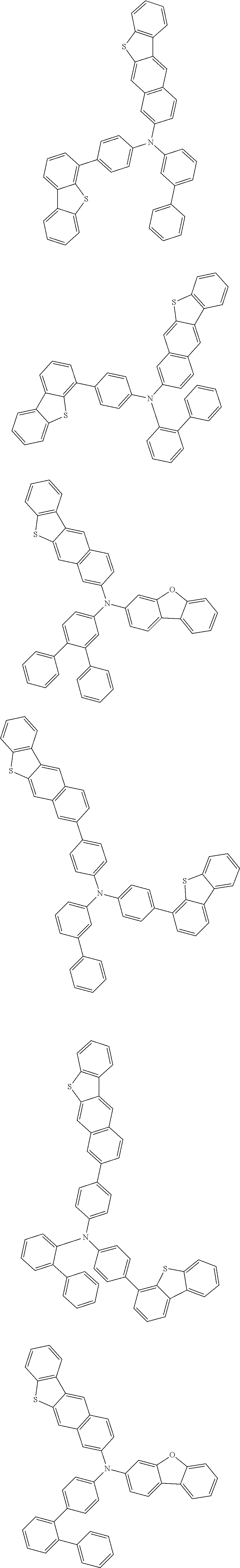

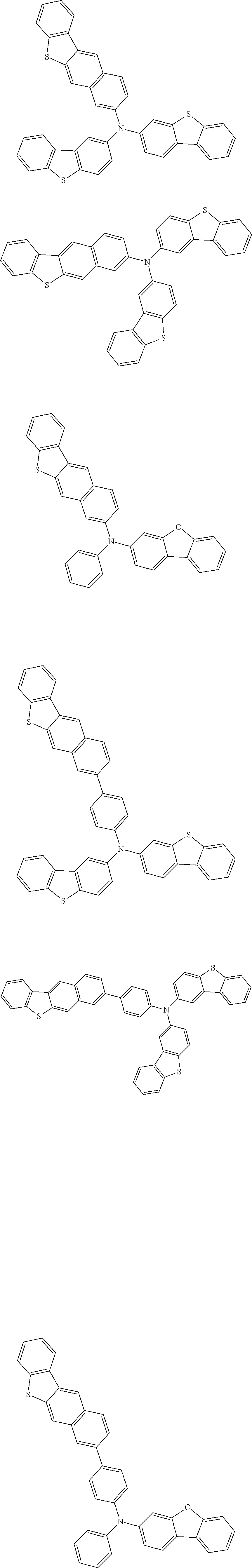 [SMILES: c1ccc2c(c1)sc1cc(N(c3ccc4cc5c(cc4c3)sc3ccccc35)c3ccc4sc5ccccc5c4c3)ccc12 ; c1ccc2c(c1)sc1ccc(N(c3ccc4cc5c(cc4c3)sc3ccccc35)c3ccc4sc5ccccc5c4c3)cc12 ; c1ccc(N(c2ccc3cc4c(cc3c2)sc2ccccc24)c2ccc3c(c2)oc2ccccc23)cc1 ; c1ccc2c(c1)sc1cc(N(c3ccc(-c4ccc5cc6c(cc5c4)sc4ccccc46)cc3)c3ccc4sc5ccccc5c4c3)ccc12 ; c1ccc2c(c1)sc1ccc(N(c3ccc(-c4ccc5cc6c(cc5c4)sc4ccccc46)cc3)c3ccc4sc5ccccc5c4c3)cc12 ; c1ccc(N(c2ccc(-c3ccc4cc5c(cc4c3)sc3ccccc35)cc2)c2ccc3c(c2)oc2ccccc23)cc1]

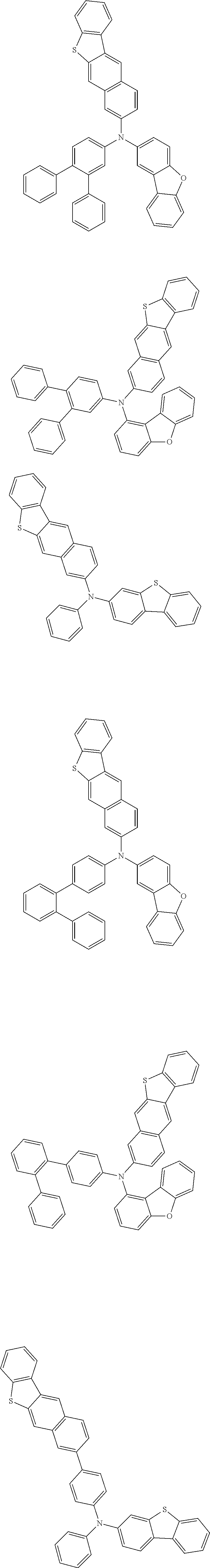

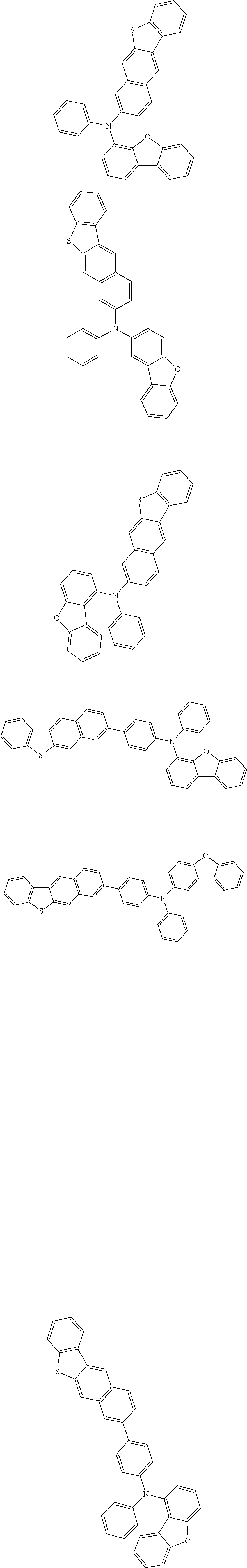

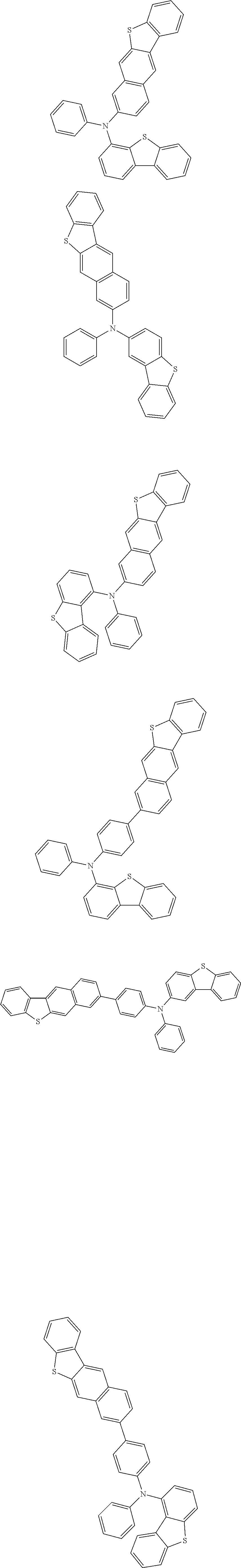

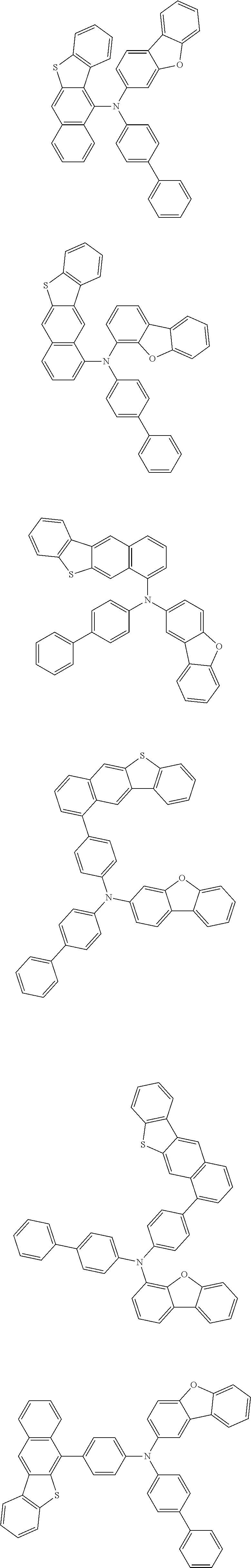 [SMILES: c1ccc(-c2ccc(N(c3ccc4c(c3)oc3ccccc34)c3c4ccccc4cc4sc5ccccc5c34)cc2)cc1 ; c1ccc(-c2ccc(N(c3cccc4cc5sc6ccccc6c5cc34)c3cccc4c3oc3ccccc34)cc2)cc1 ; c1ccc(-c2ccc(N(c3ccc4oc5ccccc5c4c3)c3cccc4cc5c(cc34)sc3ccccc35)cc2)cc1 ; c1ccc(-c2ccc(N(c3ccc(-c4cccc5cc6sc7ccccc7c6cc45)cc3)c3ccc4c(c3)oc3ccccc34)cc2)cc1 ; c1ccc(-c2ccc(N(c3ccc(-c4cccc5cc6c(cc45)sc4ccccc46)cc3)c3cccc4c3oc3ccccc34)cc2)cc1 ; c1ccc(-c2ccc(N(c3ccc(-c4c5ccccc5cc5c4sc4ccccc45)cc3)c3ccc4oc5ccccc5c4c3)cc2)cc1]

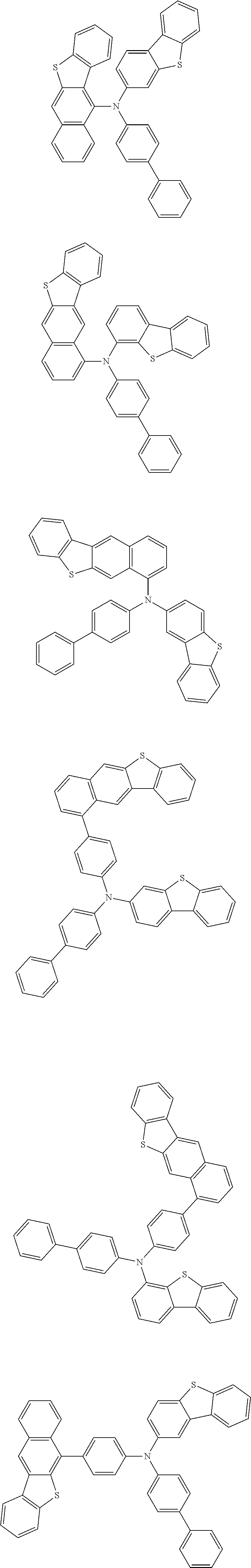 [SMILES: c1ccc(-c2ccc(N(c3ccc4c(c3)sc3ccccc34)c3c4ccccc4cc4sc5ccccc5c34)cc2)cc1 ; c1ccc(-c2ccc(N(c3cccc4cc5sc6ccccc6c5cc34)c3cccc4c3sc3ccccc34)cc2)cc1 ; c1ccc(-c2ccc(N(c3ccc4sc5ccccc5c4c3)c3cccc4cc5c(cc34)sc3ccccc35)cc2)cc1 ; c1ccc(-c2ccc(N(c3ccc(-c4cccc5cc6sc7ccccc7c6cc45)cc3)c3ccc4c(c3)sc3ccccc34)cc2)cc1 ; c1ccc(-c2ccc(N(c3ccc(-c4cccc5cc6c(cc45)sc4ccccc46)cc3)c3cccc4c3sc3ccccc34)cc2)cc1 ; c1ccc(-c2ccc(N(c3ccc(-c4c5ccccc5cc5c4sc4ccccc45)cc3)c3ccc4sc5ccccc5c4c3)cc2)cc1]

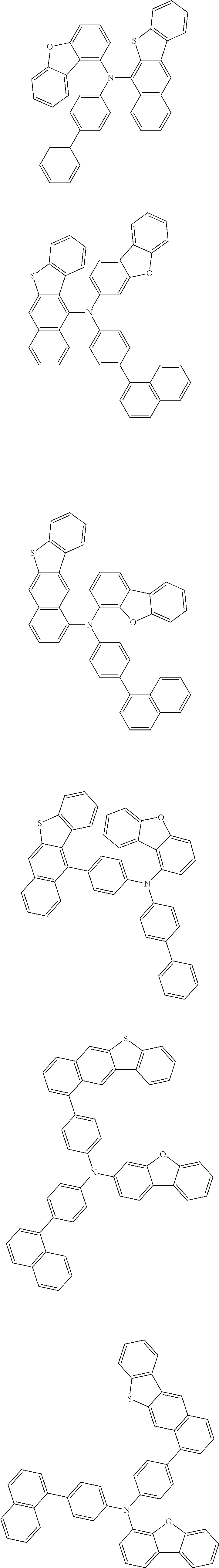 [SMILES: c1ccc(-c2ccc(N(c3c4ccccc4cc4c3sc3ccccc34)c3cccc4oc5ccccc5c34)cc2)cc1 ; c1ccc2c(-c3ccc(N(c4ccc5c(c4)oc4ccccc45)c4c5ccccc5cc5sc6ccccc6c45)cc3)cccc2c1 ; c1ccc2c(-c3ccc(N(c4cccc5cc6sc7ccccc7c6cc45)c4cccc5c4oc4ccccc45)cc3)cccc2c1 ; c1ccc(-c2ccc(N(c3ccc(-c4c5ccccc5cc5sc6ccccc6c45)cc3)c3cccc4oc5ccccc5c34)cc2)cc1 ; c1ccc2c(-c3ccc(N(c4ccc(-c5cccc6cc7sc8ccccc8c7cc56)cc4)c4ccc5c(c4)oc4ccccc45)cc3)cccc2c1 ; c1ccc2c(-c3ccc(N(c4ccc(-c5cccc6cc7c(cc56)sc5ccccc57)cc4)c4cccc5c4oc4ccccc45)cc3)cccc2c1]

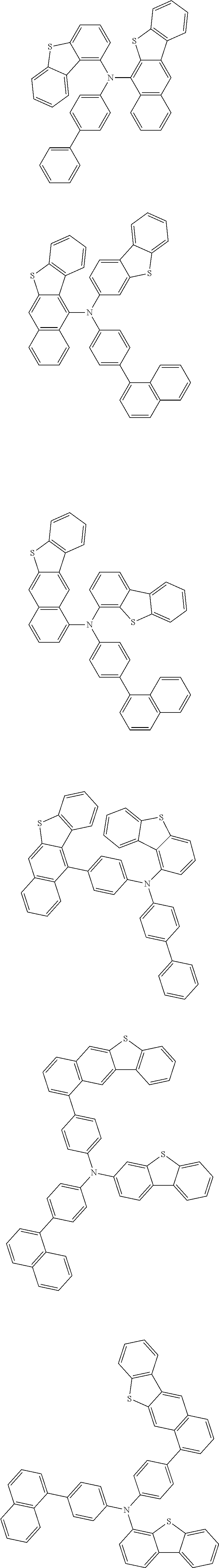 [SMILES: c1ccc(-c2ccc(N(c3c4ccccc4cc4c3sc3ccccc34)c3cccc4sc5ccccc5c34)cc2)cc1 ; c1ccc2c(-c3ccc(N(c4ccc5c(c4)sc4ccccc45)c4c5ccccc5cc5sc6ccccc6c45)cc3)cccc2c1 ; c1ccc2c(-c3ccc(N(c4cccc5cc6sc7ccccc7c6cc45)c4cccc5c4sc4ccccc45)cc3)cccc2c1 ; c1ccc(-c2ccc(N(c3ccc(-c4c5ccccc5cc5sc6ccccc6c45)cc3)c3cccc4sc5ccccc5c34)cc2)cc1 ; c1ccc2c(-c3ccc(N(c4ccc(-c5cccc6cc7sc8ccccc8c7cc56)cc4)c4ccc5c(c4)sc4ccccc45)cc3)cccc2c1 ; c1ccc2c(-c3ccc(N(c4ccc(-c5cccc6cc7c(cc56)sc5ccccc57)cc4)c4cccc5c4sc4ccccc45)cc3)cccc2c1]

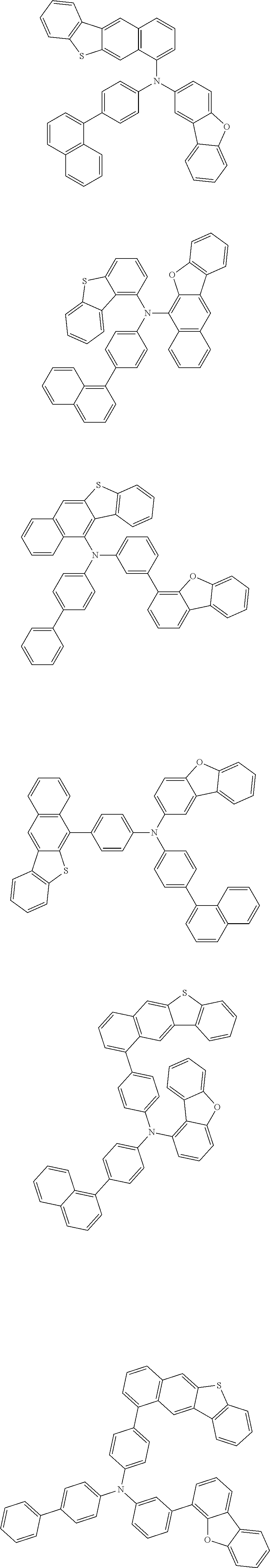

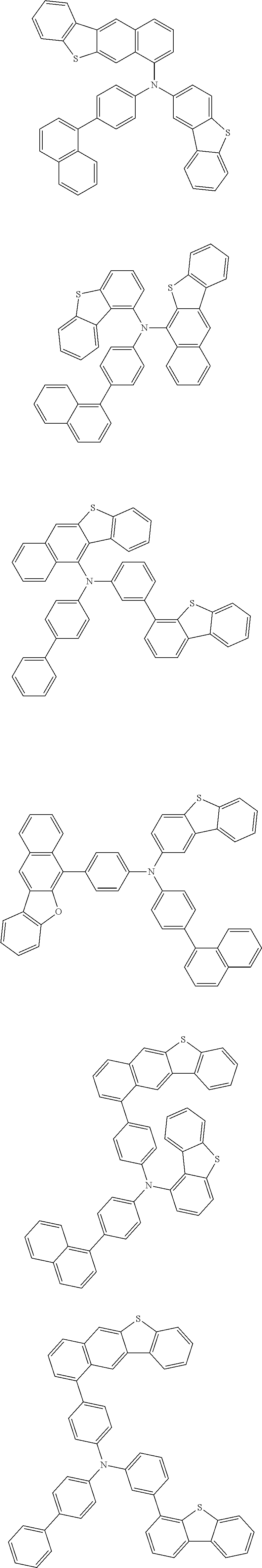

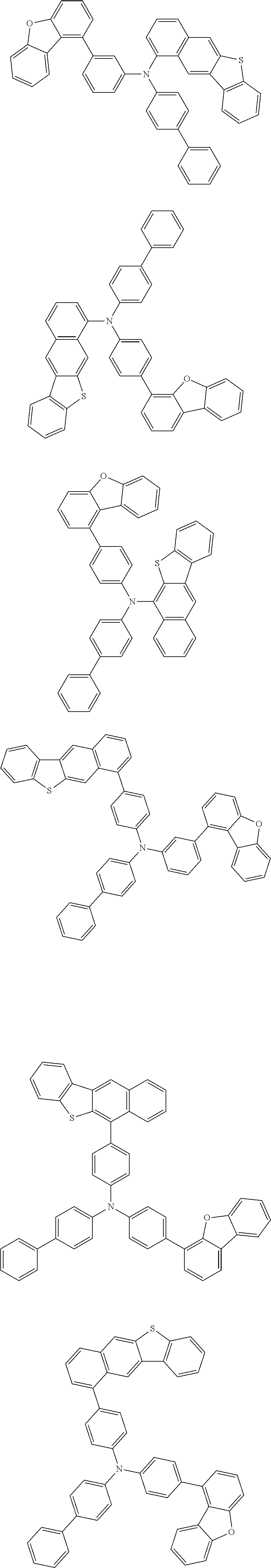

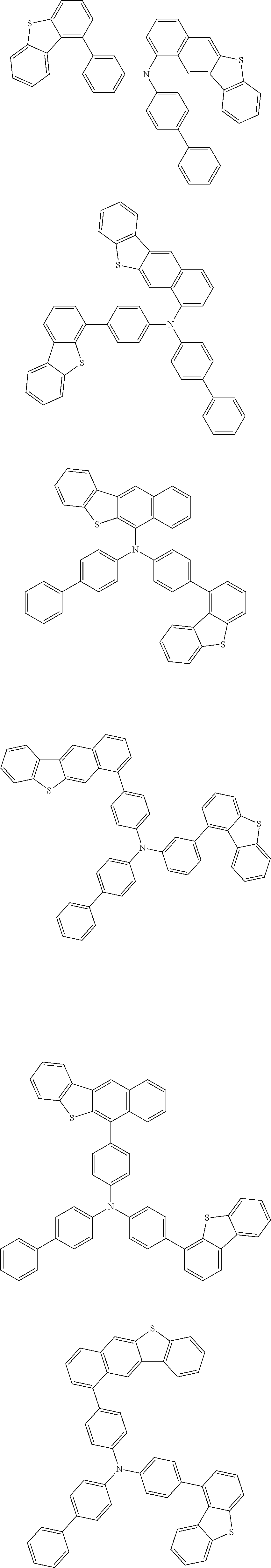

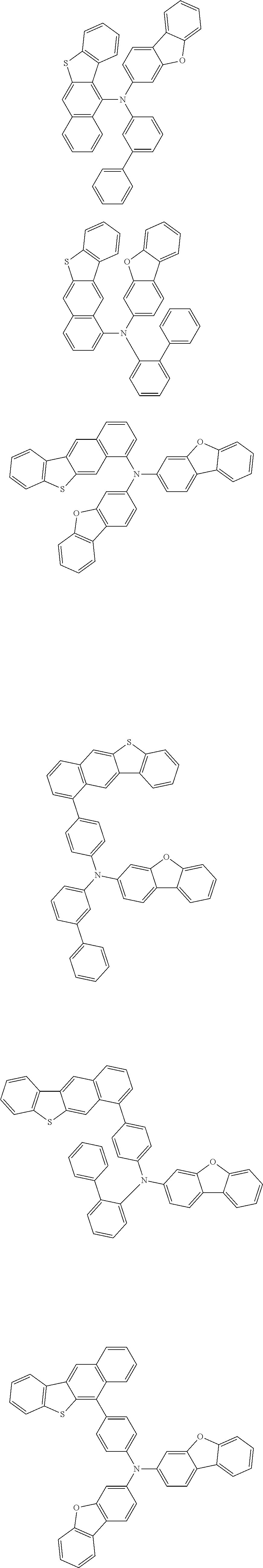

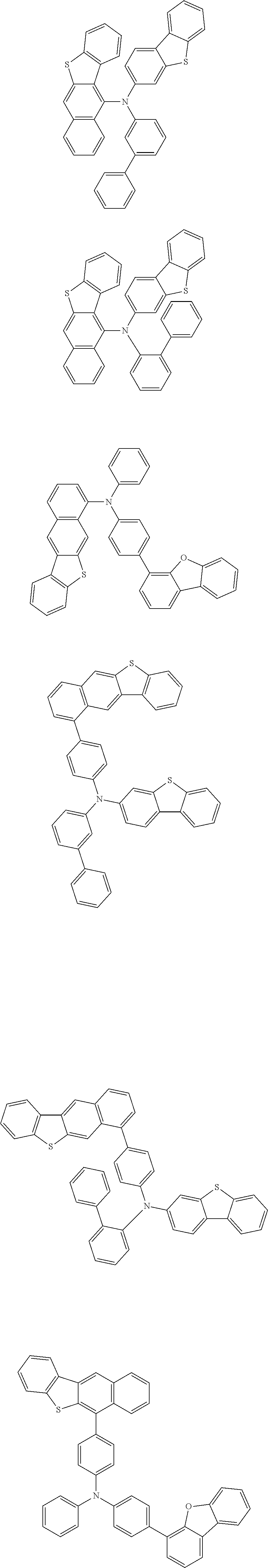 [SMILES: c1ccc(-c2cccc(N(c3ccc4c(c3)sc3ccccc34)c3c4ccccc4cc4sc5ccccc5c34)c2)cc1 ; c1ccc(-c2ccccc2N(c2ccc3c(c2)sc2ccccc23)c2c3ccccc3cc3sc4ccccc4c23)cc1 ; c1ccc(N(c2ccc(-c3cccc4c3oc3ccccc34)cc2)c2cccc3cc4c(cc23)sc2ccccc24)cc1 ; c1ccc(-c2cccc(N(c3ccc(-c4cccc5cc6sc7ccccc7c6cc45)cc3)c3ccc4c(c3)sc3ccccc34)c2)cc1 ; c1ccc(-c2ccccc2N(c2ccc(-c3cccc4cc5c(cc34)sc3ccccc35)cc2)c2ccc3c(c2)sc2ccccc23)cc1 ; c1ccc(N(c2ccc(-c3cccc4c3oc3ccccc34)cc2)c2ccc(-c3c4ccccc4cc4c3sc3ccccc34)cc2)cc1]

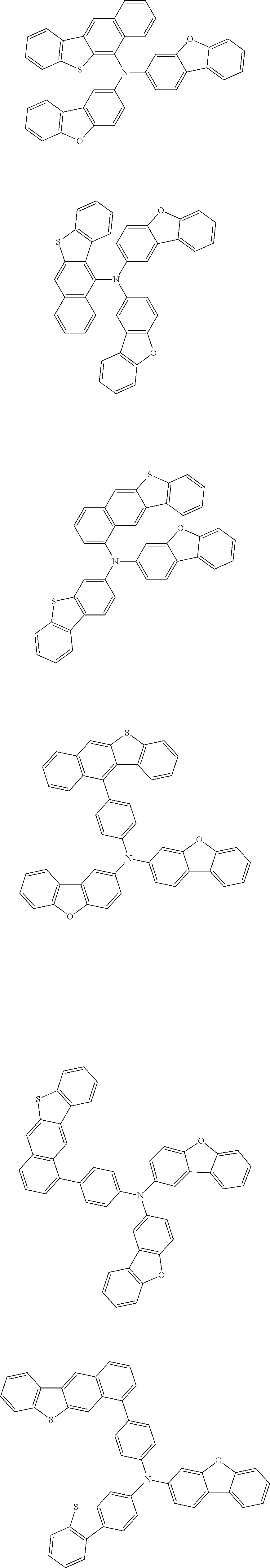 [SMILES: c1ccc2c(N(c3ccc4c(c3)oc3ccccc34)c3ccc4oc5ccccc5c4c3)c3sc4ccccc4c3cc2c1 ; c1ccc2c(N(c3ccc4oc5ccccc5c4c3)c3ccc4oc5ccccc5c4c3)c3c(cc2c1)sc1ccccc13 ; c1cc(N(c2ccc3c(c2)oc2ccccc23)c2ccc3c(c2)sc2ccccc23)c2cc3c(cc2c1)sc1ccccc13 ; c1ccc2c(-c3ccc(N(c4ccc5c(c4)oc4ccccc45)c4ccc5oc6ccccc6c5c4)cc3)c3c(cc2c1)sc1ccccc13 ; c1cc(-c2ccc(N(c3ccc4oc5ccccc5c4c3)c3ccc4oc5ccccc5c4c3)cc2)c2cc3c(cc2c1)sc1ccccc13 ; c1cc(-c2ccc(N(c3ccc4c(c3)oc3ccccc34)c3ccc4c(c3)sc3ccccc34)cc2)c2cc3sc4ccccc4c3cc2c1]

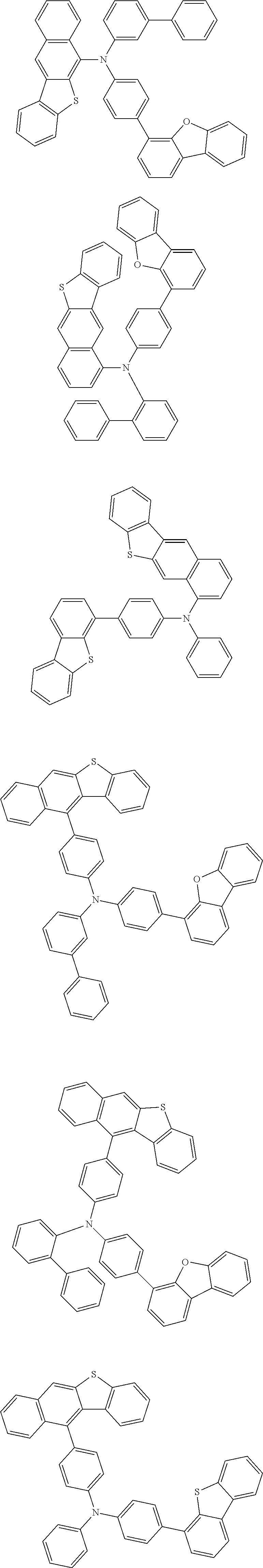

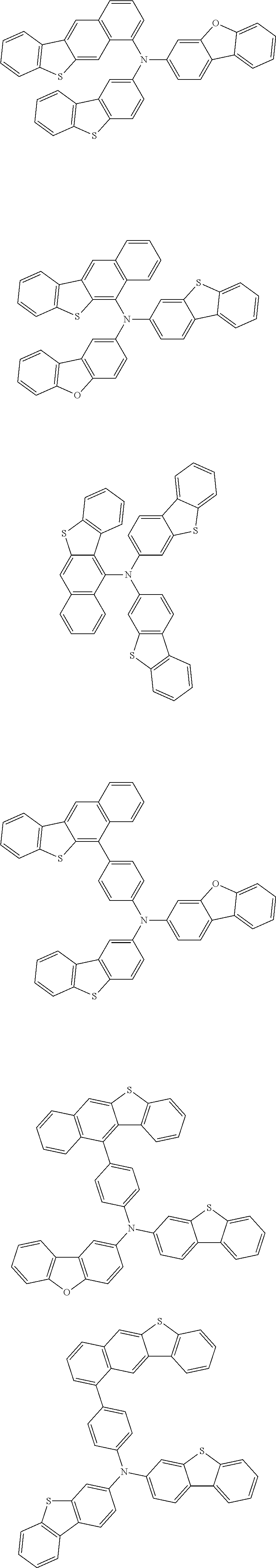 [SMILES: c1cc(N(c2ccc3c(c2)oc2ccccc23)c2ccc3sc4ccccc4c3c2)c2cc3sc4ccccc4c3cc2c1 ; c1ccc2c(N(c3ccc4c(c3)sc3ccccc34)c3ccc4oc5ccccc5c4c3)c3sc4ccccc4c3cc2c1 ; c1ccc2c(N(c3ccc4c(c3)sc3ccccc34)c3ccc4c(c3)sc3ccccc34)c3c(cc2c1)sc1ccccc13 ; c1ccc2c(-c3ccc(N(c4ccc5c(c4)oc4ccccc45)c4ccc5sc6ccccc6c5c4)cc3)c3sc4ccccc4c3cc2c1 ; c1ccc2c(-c3ccc(N(c4ccc5c(c4)sc4ccccc45)c4ccc5oc6ccccc6c5c4)cc3)c3c(cc2c1)sc1ccccc13 ; c1cc(-c2ccc(N(c3ccc4c(c3)sc3ccccc34)c3ccc4c(c3)sc3ccccc34)cc2)c2cc3c(cc2c1)sc1ccccc13]

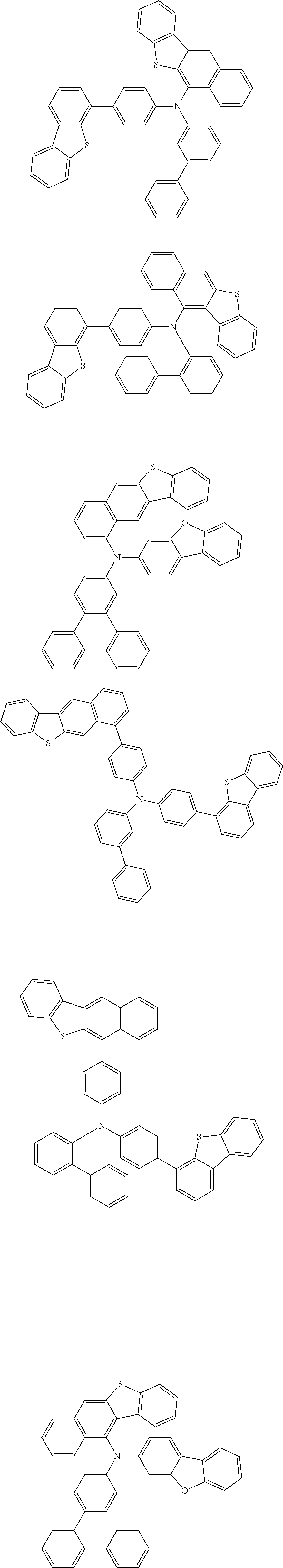

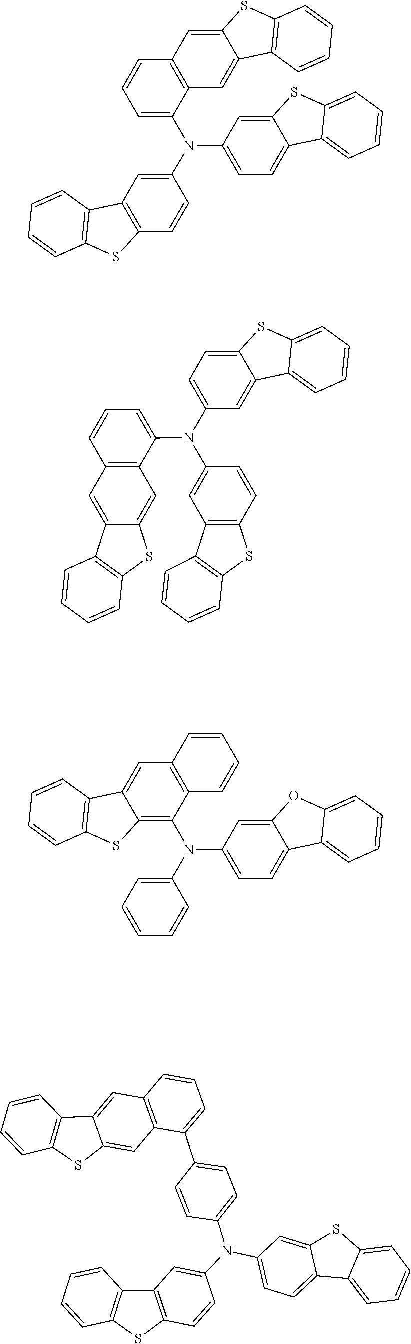 [SMILES: c1cc(N(c2ccc3c(c2)sc2ccccc23)c2ccc3sc4ccccc4c3c2)c2cc3c(cc2c1)sc1ccccc13 ; c1cc(N(c2ccc3sc4ccccc4c3c2)c2ccc3sc4ccccc4c3c2)c2cc3sc4ccccc4c3cc2c1 ; c1ccc(N(c2ccc3c(c2)oc2ccccc23)c2c3ccccc3cc3c2sc2ccccc23)cc1 ; c1cc(-c2ccc(N(c3ccc4c(c3)sc3ccccc34)c3ccc4sc5ccccc5c4c3)cc2)c2cc3sc4ccccc4c3cc2c1]

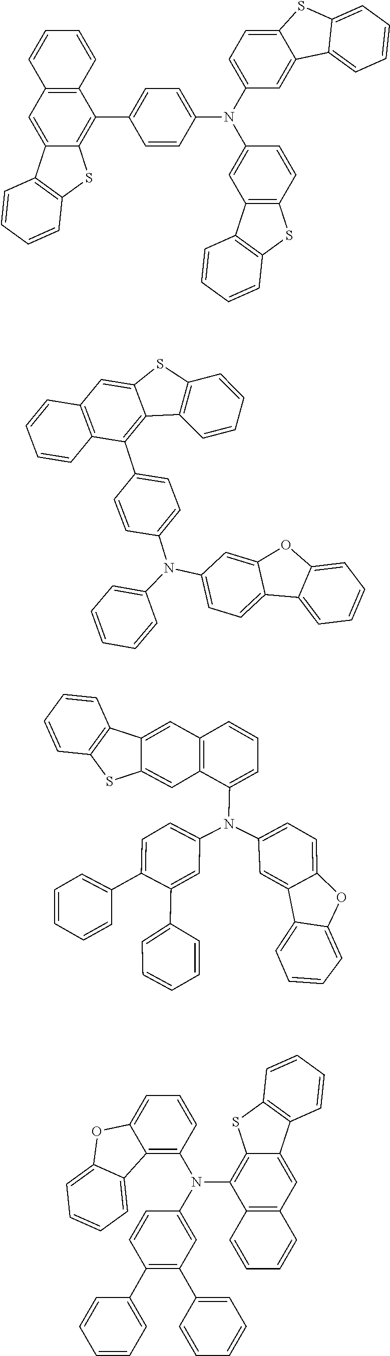 [SMILES: c1ccc2c(-c3ccc(N(c4ccc5sc6ccccc6c5c4)c4ccc5sc6ccccc6c5c4)cc3)c3sc4ccccc4c3cc2c1 ; c1ccc(N(c2ccc(-c3c4ccccc4cc4sc5ccccc5c34)cc2)c2ccc3c(c2)oc2ccccc23)cc1 ; c1ccc(-c2ccc(N(c3ccc4oc5ccccc5c4c3)c3cccc4cc5c(cc34)sc3ccccc35)cc2-c2ccccc2)cc1 ; c1ccc(-c2ccc(N(c3c4ccccc4cc4c3sc3ccccc34)c3cccc4oc5ccccc5c34)cc2-c2ccccc2)cc1]

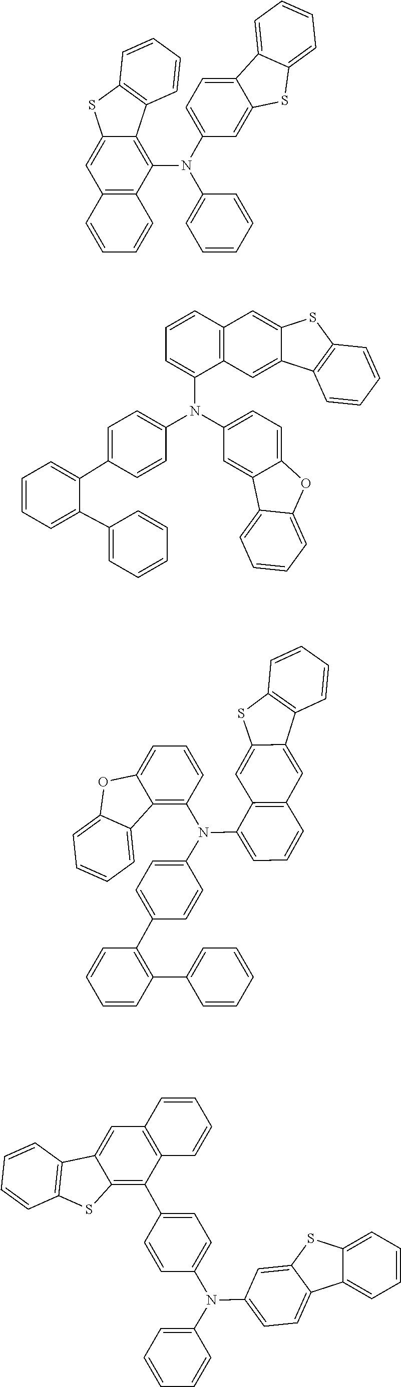 [SMILES: c1ccc(N(c2ccc3c(c2)sc2ccccc23)c2c3ccccc3cc3sc4ccccc4c23)cc1 ; c1ccc(-c2ccccc2-c2ccc(N(c3ccc4oc5ccccc5c4c3)c3cccc4cc5sc6ccccc6c5cc34)cc2)cc1 ; c1ccc(-c2ccccc2-c2ccc(N(c3cccc4cc5c(cc34)sc3ccccc35)c3cccc4oc5ccccc5c34)cc2)cc1 ; c1ccc(N(c2ccc(-c3c4ccccc4cc4c3sc3ccccc34)cc2)c2ccc3c(c2)sc2ccccc23)cc1]

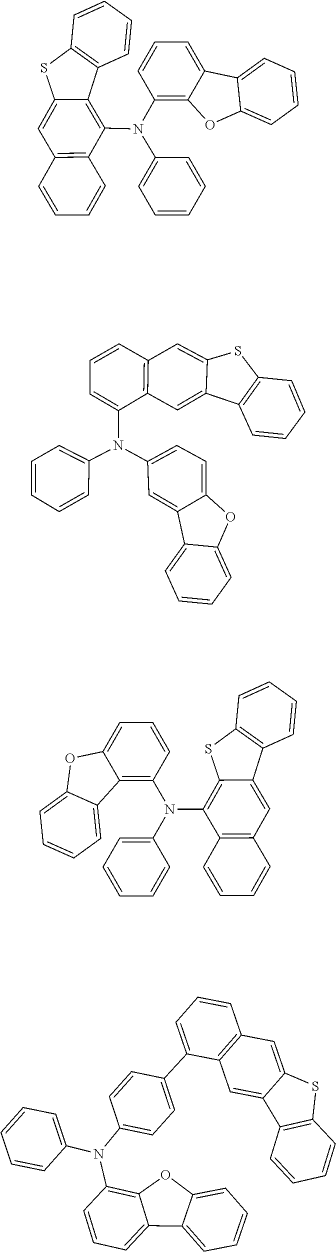 [SMILES: c1ccc(N(c2cccc3c2oc2ccccc23)c2c3ccccc3cc3sc4ccccc4c23)cc1 ; c1ccc(N(c2ccc3oc4ccccc4c3c2)c2cccc3cc4sc5ccccc5c4cc23)cc1 ; c1ccc(N(c2c3ccccc3cc3c2sc2ccccc23)c2cccc3oc4ccccc4c23)cc1 ; c1ccc(N(c2ccc(-c3cccc4cc5sc6ccccc6c5cc34)cc2)c2cccc3c2oc2ccccc23)cc1]

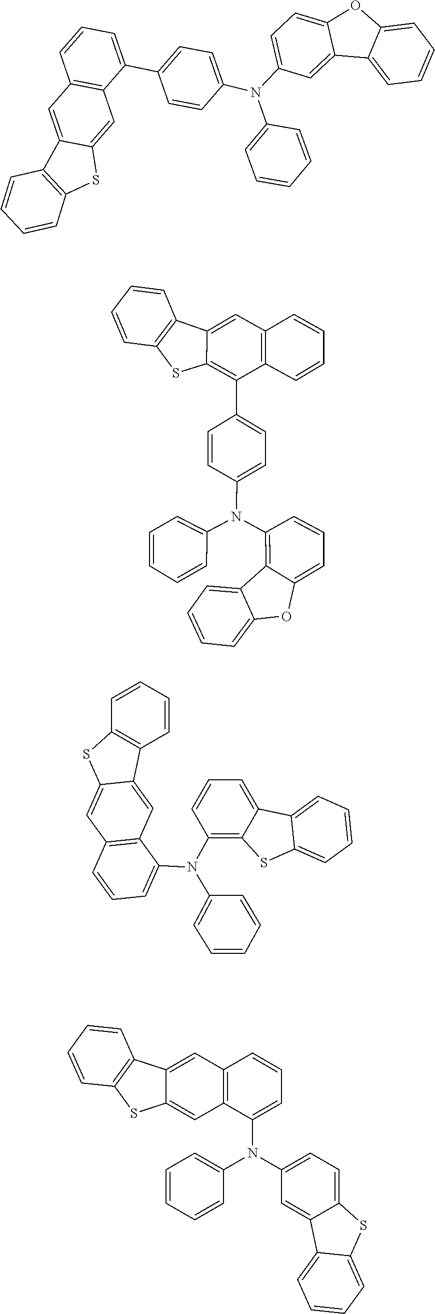 [SMILES: c1ccc(N(c2ccc(-c3cccc4cc5c(cc34)sc3ccccc35)cc2)c2ccc3oc4ccccc4c3c2)cc1 ; c1ccc(N(c2ccc(-c3c4ccccc4cc4c3sc3ccccc34)cc2)c2cccc3oc4ccccc4c23)cc1 ; c1ccc(N(c2cccc3cc4sc5ccccc5c4cc23)c2cccc3c2sc2ccccc23)cc1 ; c1ccc(N(c2ccc3sc4ccccc4c3c2)c2cccc3cc4c(cc23)sc2ccccc24)cc1]

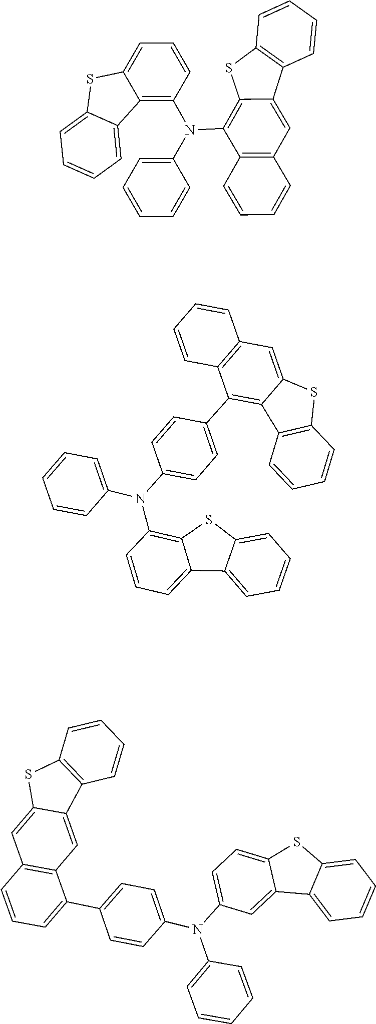 [SMILES: c1ccc(N(c2c3ccccc3cc3c2sc2ccccc23)c2cccc3sc4ccccc4c23)cc1 ; c1ccc(N(c2ccc(-c3c4ccccc4cc4sc5ccccc5c34)cc2)c2cccc3c2sc2ccccc23)cc1 ; c1ccc(N(c2ccc(-c3cccc4cc5sc6ccccc6c5cc34)cc2)c2ccc3sc4ccccc4c3c2)cc1]

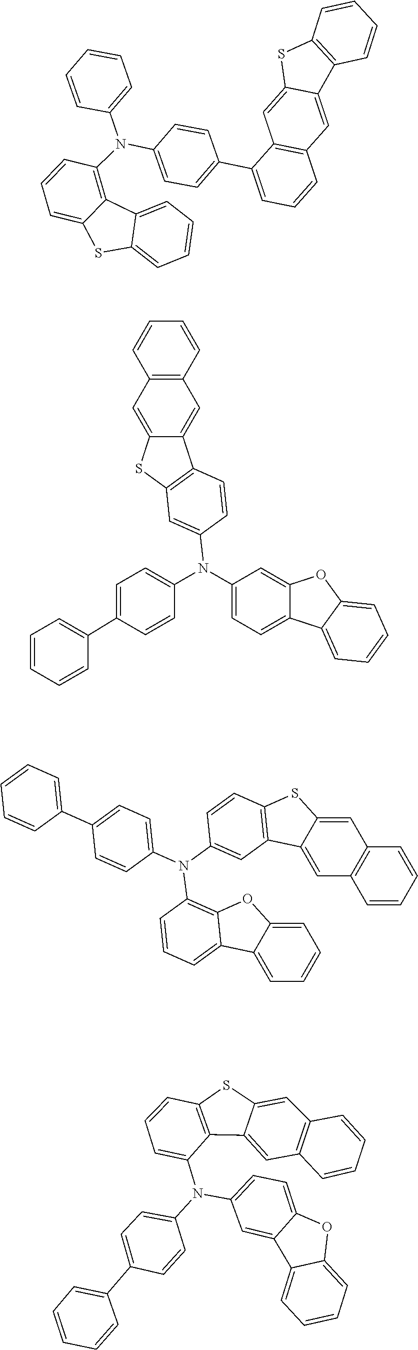 [SMILES: c1ccc(N(c2ccc(-c3cccc4cc5c(cc34)sc3ccccc35)cc2)c2cccc3sc4ccccc4c23)cc1 ; c1ccc(-c2ccc(N(c3ccc4c(c3)oc3ccccc34)c3ccc4c(c3)sc3cc5ccccc5cc34)cc2)cc1 ; c1ccc(-c2ccc(N(c3ccc4sc5cc6ccccc6cc5c4c3)c3cccc4c3oc3ccccc34)cc2)cc1 ; c1ccc(-c2ccc(N(c3ccc4oc5ccccc5c4c3)c3cccc4sc5cc6ccccc6cc5c34)cc2)cc1]

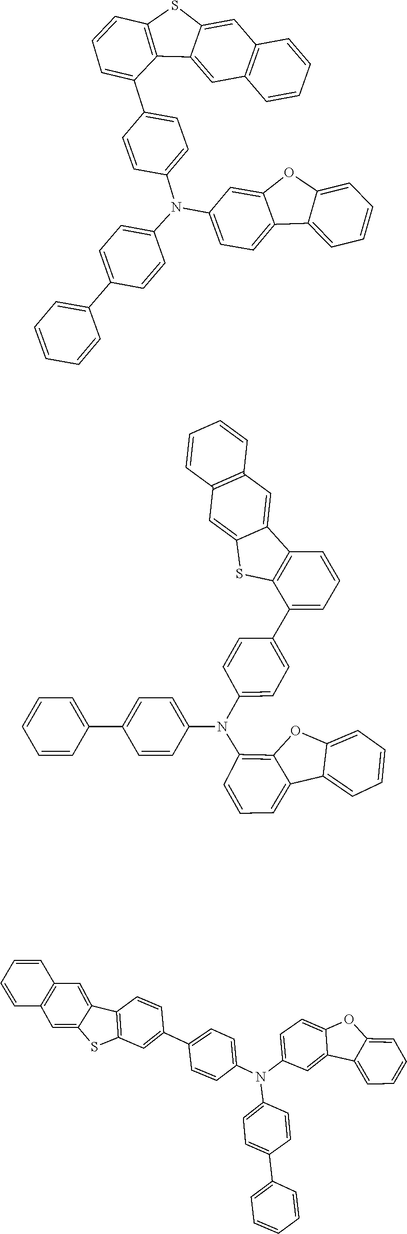 [SMILES: c1ccc(-c2ccc(N(c3ccc(-c4cccc5sc6cc7ccccc7cc6c45)cc3)c3ccc4c(c3)oc3ccccc34)cc2)cc1 ; c1ccc(-c2ccc(N(c3ccc(-c4cccc5c4sc4cc6ccccc6cc45)cc3)c3cccc4c3oc3ccccc34)cc2)cc1 ; c1ccc(-c2ccc(N(c3ccc(-c4ccc5c(c4)sc4cc6ccccc6cc45)cc3)c3ccc4oc5ccccc5c4c3)cc2)cc1]

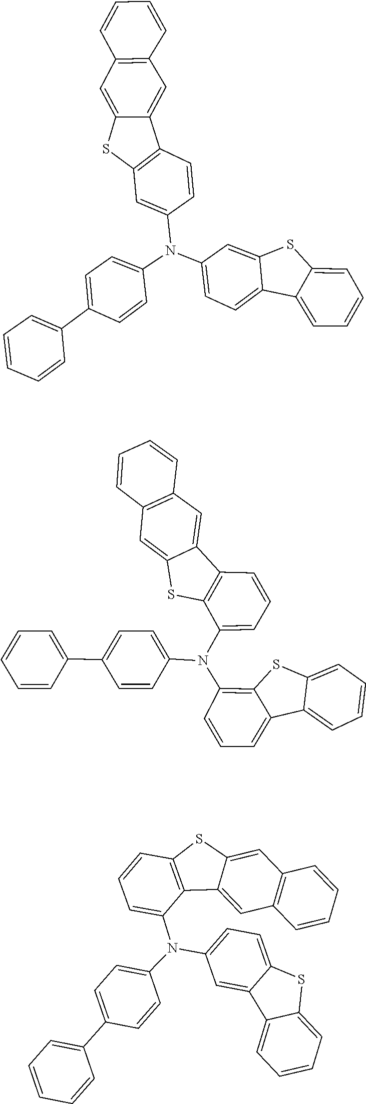 [SMILES: c1ccc(-c2ccc(N(c3ccc4c(c3)sc3ccccc34)c3ccc4c(c3)sc3cc5ccccc5cc34)cc2)cc1 ; c1ccc(-c2ccc(N(c3cccc4c3sc3ccccc34)c3cccc4c3sc3cc5ccccc5cc34)cc2)cc1 ; c1ccc(-c2ccc(N(c3ccc4sc5ccccc5c4c3)c3cccc4sc5cc6ccccc6cc5c34)cc2)cc1]

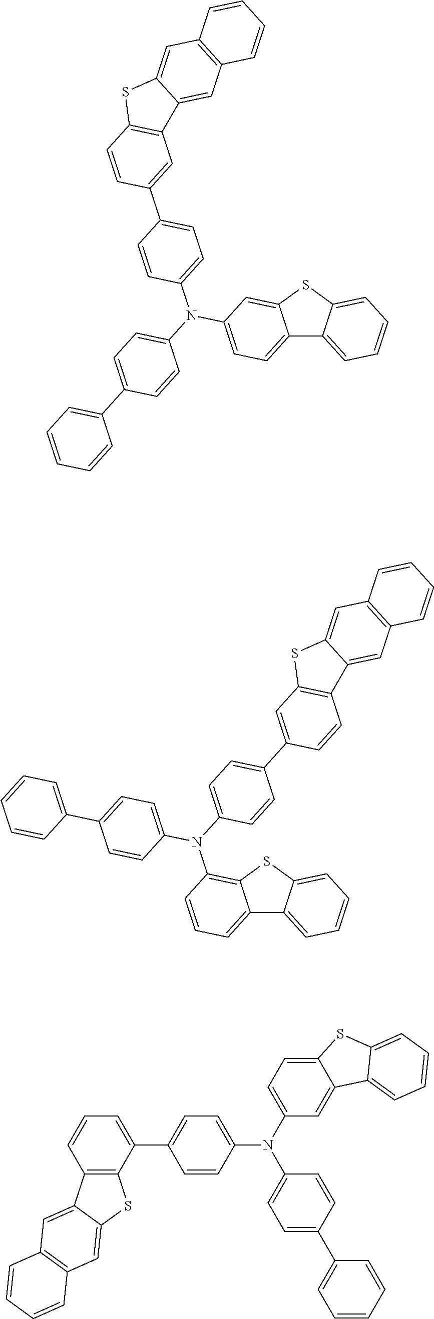 [SMILES: c1ccc(-c2ccc(N(c3ccc(-c4ccc5sc6cc7ccccc7cc6c5c4)cc3)c3ccc4c(c3)sc3ccccc34)cc2)cc1 ; c1ccc(-c2ccc(N(c3ccc(-c4ccc5c(c4)sc4cc6ccccc6cc45)cc3)c3cccc4c3sc3ccccc34)cc2)cc1 ; c1ccc(-c2ccc(N(c3ccc(-c4cccc5c4sc4cc6ccccc6cc45)cc3)c3ccc4sc5ccccc5c4c3)cc2)cc1]

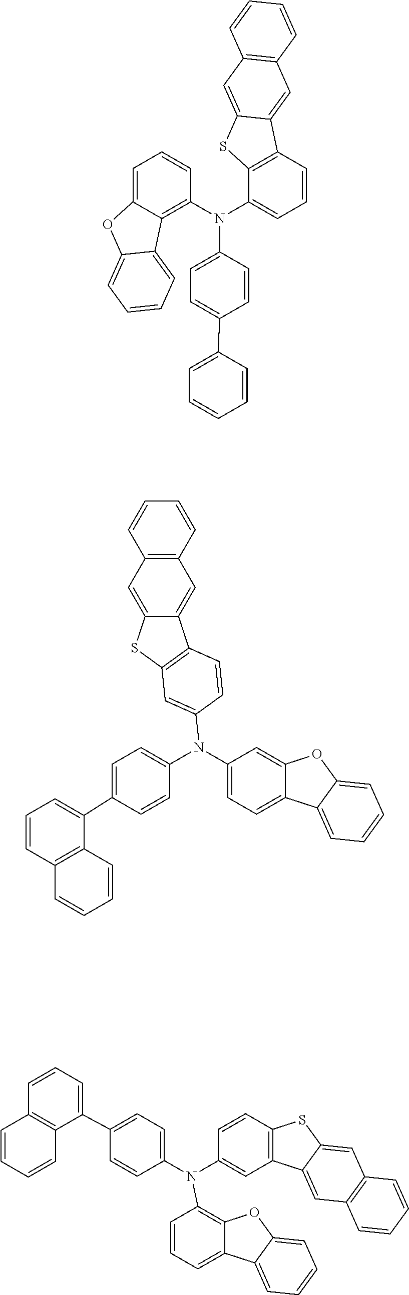 [SMILES: c1ccc(-c2ccc(N(c3cccc4c3sc3cc5ccccc5cc34)c3cccc4oc5ccccc5c34)cc2)cc1 ; c1ccc2cc3c(cc2c1)sc1cc(N(c2ccc(-c4cccc5ccccc45)cc2)c2ccc4c(c2)oc2ccccc24)ccc13 ; c1ccc2cc3c(cc2c1)sc1ccc(N(c2ccc(-c4cccc5ccccc45)cc2)c2cccc4c2oc2ccccc24)cc13]

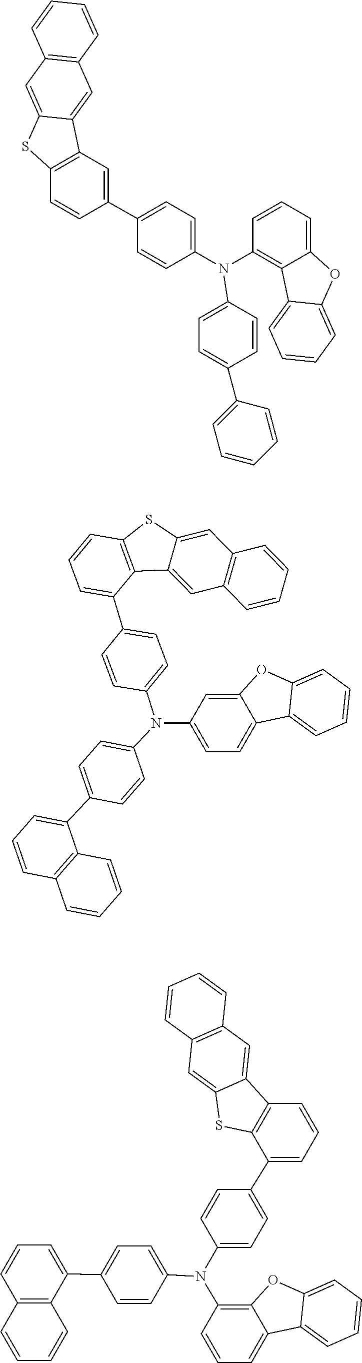 [SMILES: c1ccc(-c2ccc(N(c3ccc(-c4ccc5sc6cc7ccccc7cc6c5c4)cc3)c3cccc4oc5ccccc5c34)cc2)cc1 ; c1ccc2cc3c(cc2c1)sc1cccc(-c2ccc(N(c4ccc(-c5cccc6ccccc56)cc4)c4ccc5c(c4)oc4ccccc45)cc2)c13 ; c1ccc2cc3c(cc2c1)sc1c(-c2ccc(N(c4ccc(-c5cccc6ccccc56)cc4)c4cccc5c4oc4ccccc45)cc2)cccc13]

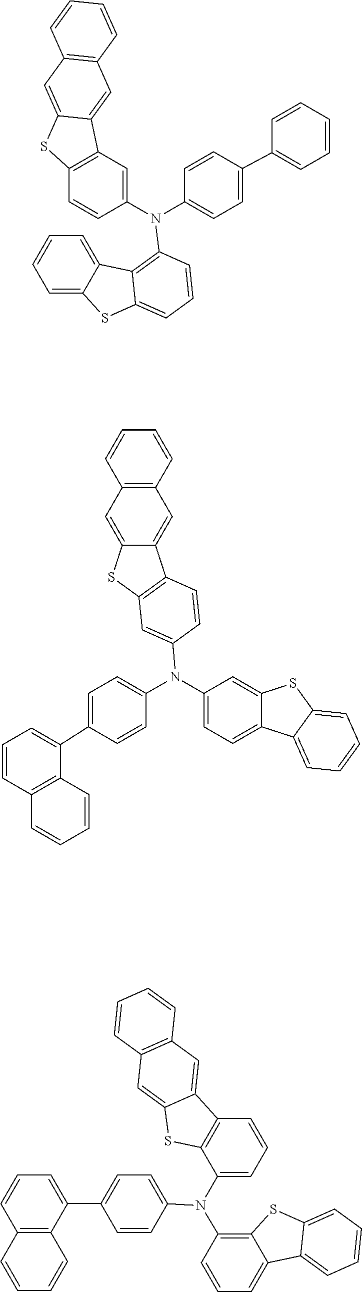 [SMILES: c1ccc(-c2ccc(N(c3ccc4sc5cc6ccccc6cc5c4c3)c3cccc4sc5ccccc5c34)cc2)cc1 ; c1ccc2cc3c(cc2c1)sc1cc(N(c2ccc(-c4cccc5ccccc45)cc2)c2ccc4c(c2)sc2ccccc24)ccc13 ; c1ccc2cc3c(cc2c1)sc1c(N(c2ccc(-c4cccc5ccccc45)cc2)c2cccc4c2sc2ccccc24)cccc13]

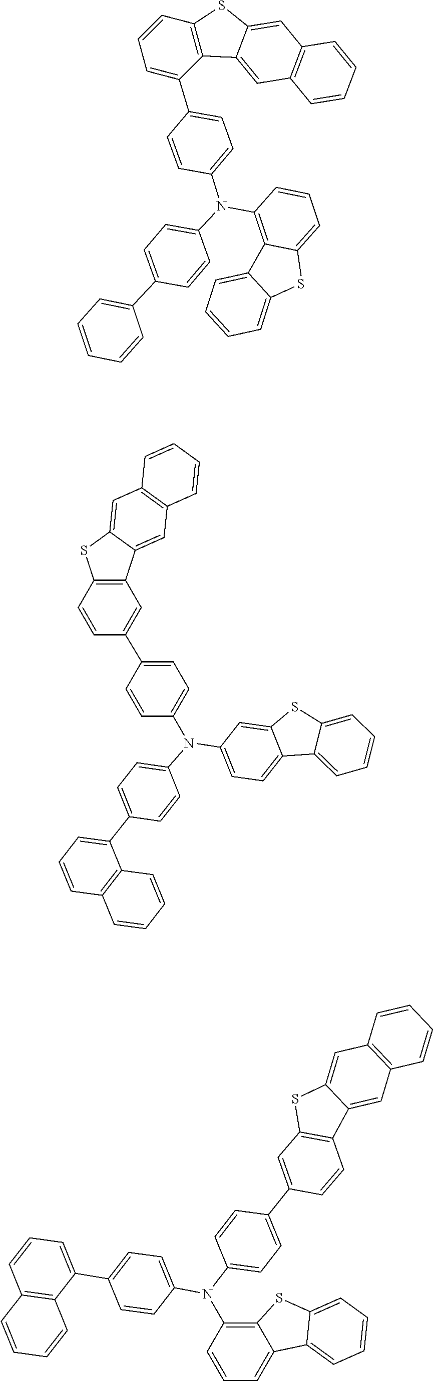 [SMILES: c1ccc(-c2ccc(N(c3ccc(-c4cccc5sc6cc7ccccc7cc6c45)cc3)c3cccc4sc5ccccc5c34)cc2)cc1 ; c1ccc2cc3c(cc2c1)sc1ccc(-c2ccc(N(c4ccc(-c5cccc6ccccc56)cc4)c4ccc5c(c4)sc4ccccc45)cc2)cc13 ; c1ccc2cc3c(cc2c1)sc1cc(-c2ccc(N(c4ccc(-c5cccc6ccccc56)cc4)c4cccc5c4sc4ccccc45)cc2)ccc13]

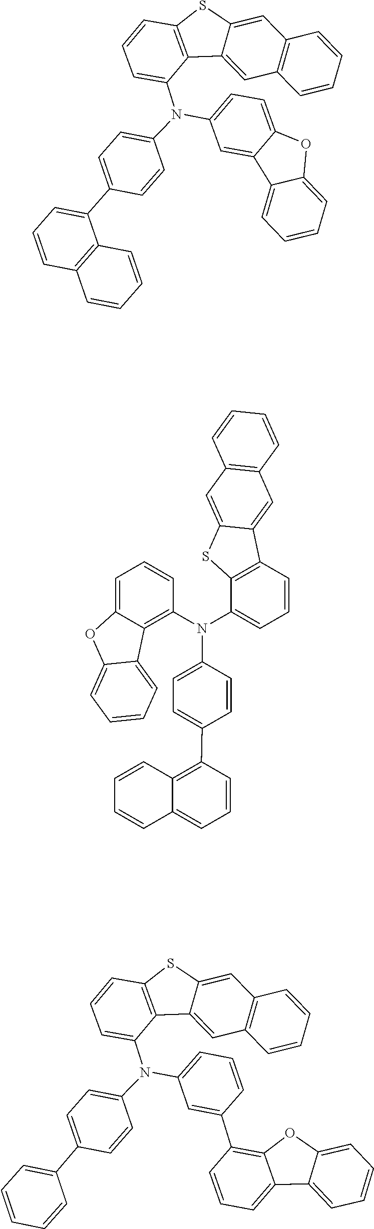 [SMILES: c1ccc2cc3c(cc2c1)sc1cccc(N(c2ccc(-c4cccc5ccccc45)cc2)c2ccc4oc5ccccc5c4c2)c13 ; c1ccc2cc3c(cc2c1)sc1c(N(c2ccc(-c4cccc5ccccc45)cc2)c2cccc4oc5ccccc5c24)cccc13 ; c1ccc(-c2ccc(N(c3cccc(-c4cccc5c4oc4ccccc45)c3)c3cccc4sc5cc6ccccc6cc5c34)cc2)cc1]

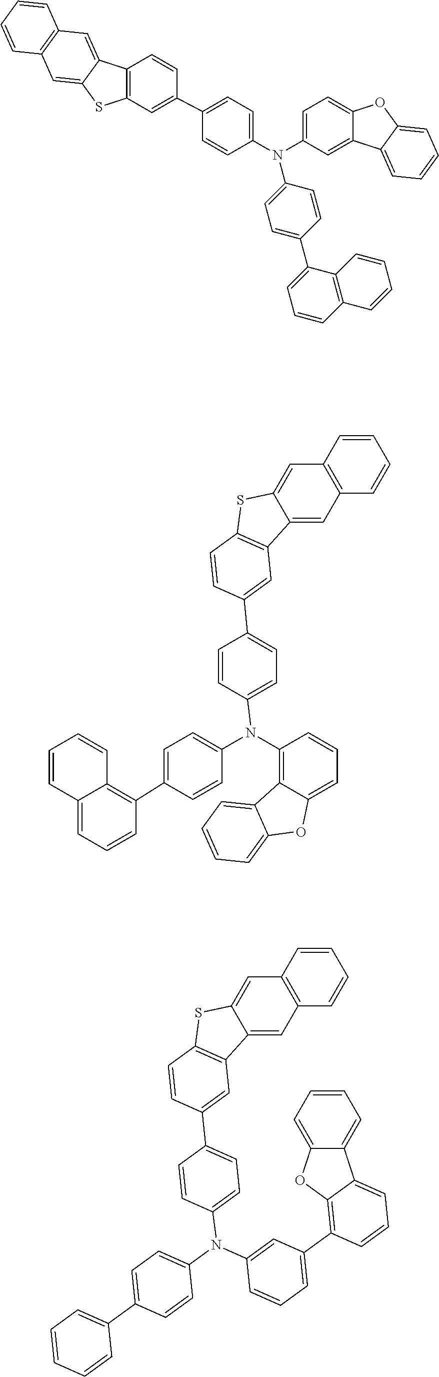 [SMILES: c1ccc2cc3c(cc2c1)sc1cc(-c2ccc(N(c4ccc(-c5cccc6ccccc56)cc4)c4ccc5oc6ccccc6c5c4)cc2)ccc13 ; c1ccc2cc3c(cc2c1)sc1ccc(-c2ccc(N(c4ccc(-c5cccc6ccccc56)cc4)c4cccc5oc6ccccc6c45)cc2)cc13 ; c1ccc(-c2ccc(N(c3ccc(-c4ccc5sc6cc7ccccc7cc6c5c4)cc3)c3cccc(-c4cccc5c4oc4ccccc45)c3)cc2)cc1]

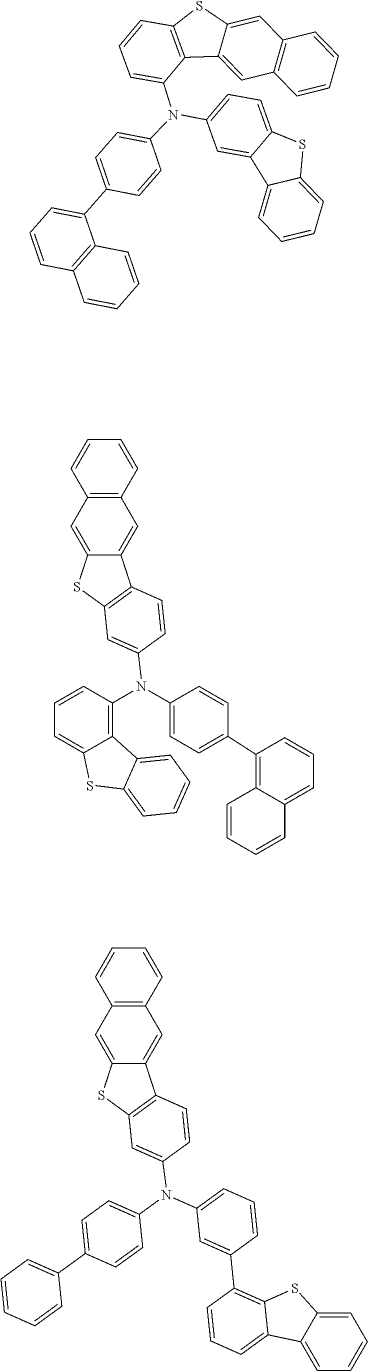 [SMILES: c1ccc2cc3c(cc2c1)sc1cccc(N(c2ccc(-c4cccc5ccccc45)cc2)c2ccc4sc5ccccc5c4c2)c13 ; c1ccc2cc3c(cc2c1)sc1cc(N(c2ccc(-c4cccc5ccccc45)cc2)c2cccc4sc5ccccc5c24)ccc13 ; c1ccc(-c2ccc(N(c3cccc(-c4cccc5c4sc4ccccc45)c3)c3ccc4c(c3)sc3cc5ccccc5cc34)cc2)cc1]

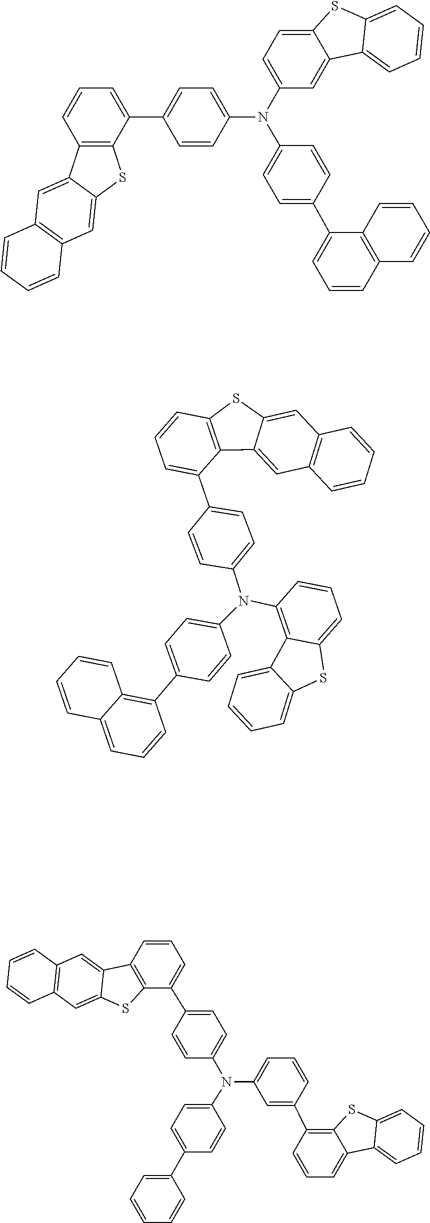 [SMILES: c1ccc2cc3c(cc2c1)sc1c(-c2ccc(N(c4ccc(-c5cccc6ccccc56)cc4)c4ccc5sc6ccccc6c5c4)cc2)cccc13 ; c1ccc2cc3c(cc2c1)sc1cccc(-c2ccc(N(c4ccc(-c5cccc6ccccc56)cc4)c4cccc5sc6ccccc6c45)cc2)c13 ; c1ccc(-c2ccc(N(c3ccc(-c4cccc5c4sc4cc6ccccc6cc45)cc3)c3cccc(-c4cccc5c4sc4ccccc45)c3)cc2)cc1]

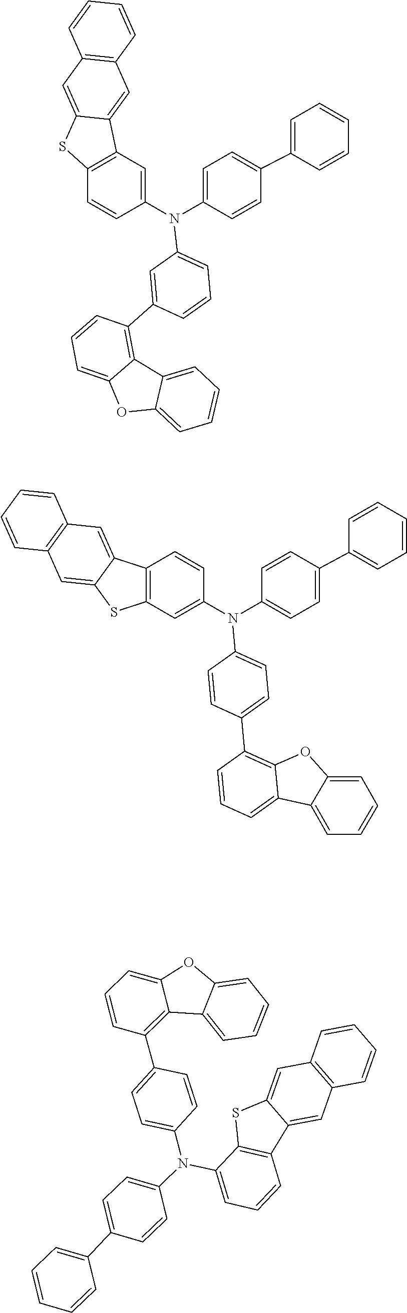 [SMILES: c1ccc(-c2ccc(N(c3cccc(-c4cccc5oc6ccccc6c45)c3)c3ccc4sc5cc6ccccc6cc5c4c3)cc2)cc1 ; c1ccc(-c2ccc(N(c3ccc(-c4cccc5c4oc4ccccc45)cc3)c3ccc4c(c3)sc3cc5ccccc5cc34)cc2)cc1 ; c1ccc(-c2ccc(N(c3ccc(-c4cccc5oc6ccccc6c45)cc3)c3cccc4c3sc3cc5ccccc5cc34)cc2)cc1]

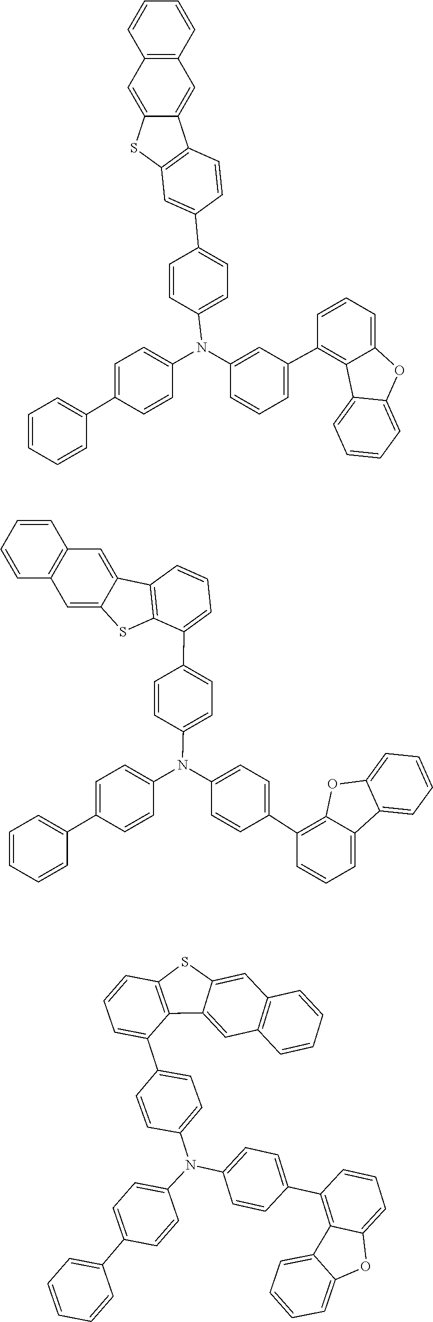 [SMILES: c1ccc(-c2ccc(N(c3ccc(-c4ccc5c(c4)sc4cc6ccccc6cc45)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4cccc5c4sc4cc6ccccc6cc45)cc3)cc2)cc1 ; c1ccc(-c2ccc(N(c3ccc(-c4cccc5oc6ccccc6c45)cc3)c3ccc(-c4cccc5sc6cc7ccccc7cc6c45)cc3)cc2)cc1]

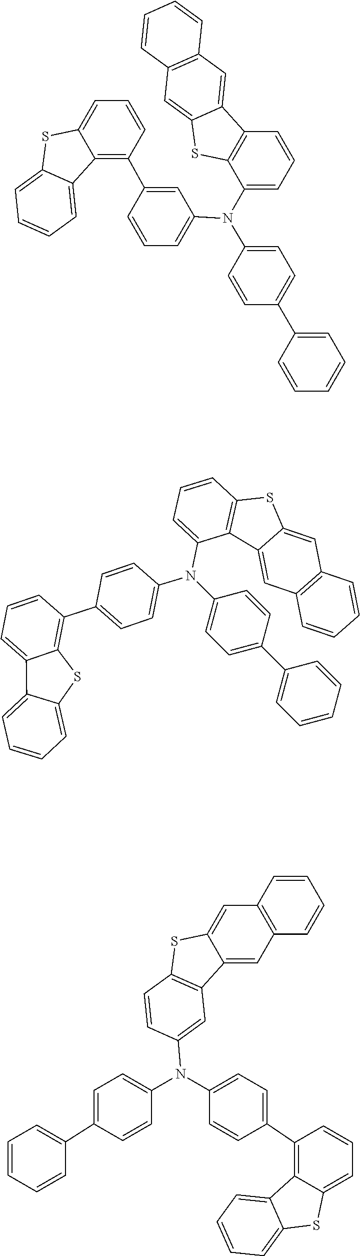 [SMILES: c1ccc(-c2ccc(N(c3cccc(-c4cccc5sc6ccccc6c45)c3)c3cccc4c3sc3cc5ccccc5cc34)cc2)cc1 ; c1ccc(-c2ccc(N(c3ccc(-c4cccc5c4sc4ccccc45)cc3)c3cccc4sc5cc6ccccc6cc5c34)cc2)cc1 ; c1ccc(-c2ccc(N(c3ccc(-c4cccc5sc6ccccc6c45)cc3)c3ccc4sc5cc6ccccc6cc5c4c3)cc2)cc1]

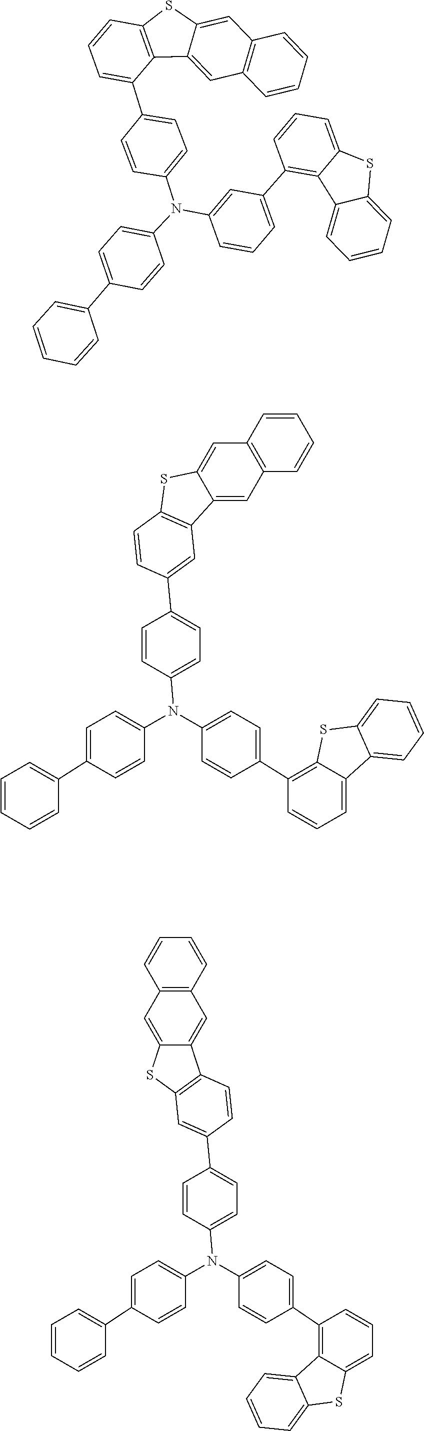 [SMILES: c1ccc(-c2ccc(N(c3ccc(-c4cccc5sc6cc7ccccc7cc6c45)cc3)c3cccc(-c4cccc5sc6ccccc6c45)c3)cc2)cc1 ; c1ccc(-c2ccc(N(c3ccc(-c4ccc5sc6cc7ccccc7cc6c5c4)cc3)c3ccc(-c4cccc5c4sc4ccccc45)cc3)cc2)cc1 ; c1ccc(-c2ccc(N(c3ccc(-c4ccc5c(c4)sc4cc6ccccc6cc45)cc3)c3ccc(-c4cccc5sc6ccccc6c45)cc3)cc2)cc1]

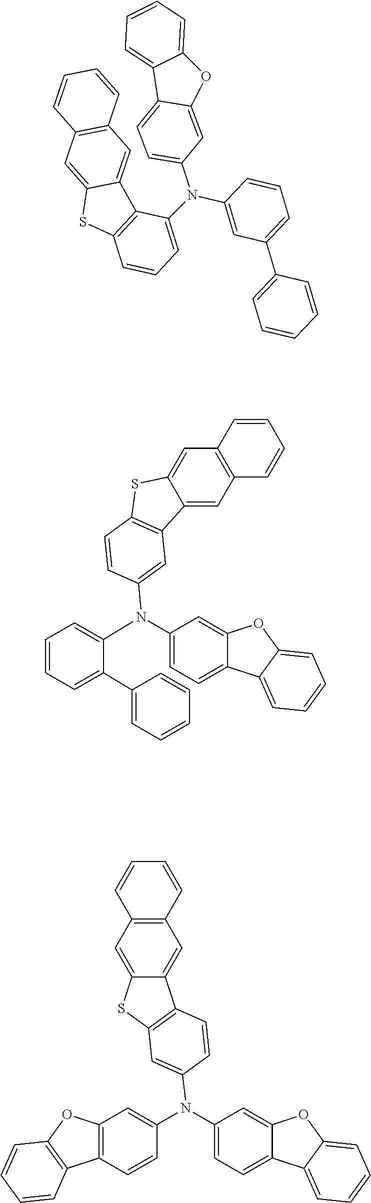 [SMILES: c1ccc(-c2cccc(N(c3ccc4c(c3)oc3ccccc34)c3cccc4sc5cc6ccccc6cc5c34)c2)cc1 ; c1ccc(-c2ccccc2N(c2ccc3c(c2)oc2ccccc23)c2ccc3sc4cc5ccccc5cc4c3c2)cc1 ; c1ccc2cc3c(cc2c1)sc1cc(N(c2ccc4c(c2)oc2ccccc24)c2ccc4c(c2)oc2ccccc24)ccc13]

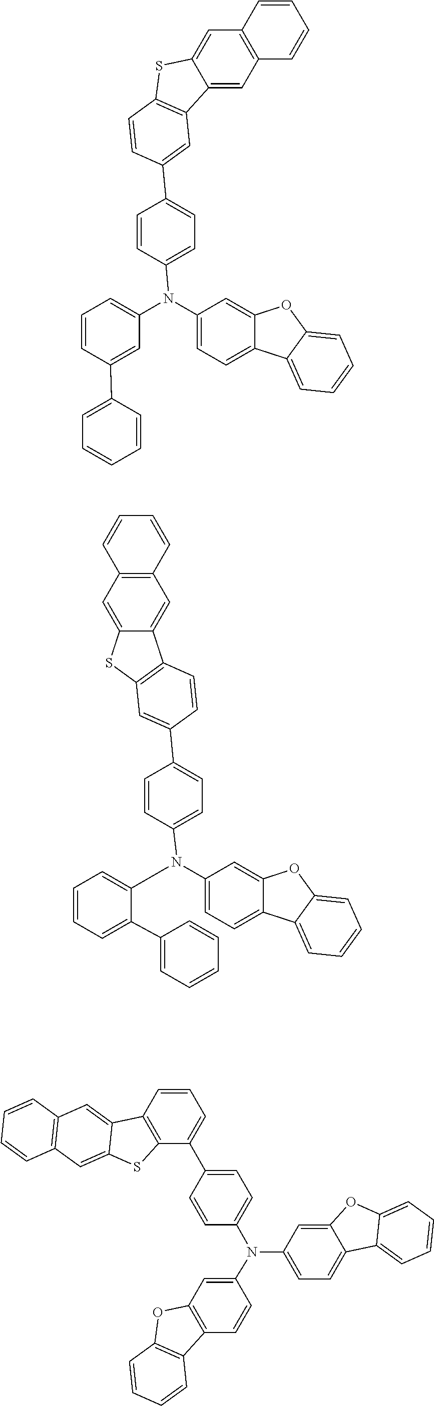 [SMILES: c1ccc(-c2cccc(N(c3ccc(-c4ccc5sc6cc7ccccc7cc6c5c4)cc3)c3ccc4c(c3)oc3ccccc34)c2)cc1 ; c1ccc(-c2ccccc2N(c2ccc(-c3ccc4c(c3)sc3cc5ccccc5cc34)cc2)c2ccc3c(c2)oc2ccccc23)cc1 ; c1ccc2cc3c(cc2c1)sc1c(-c2ccc(N(c4ccc5c(c4)oc4ccccc45)c4ccc5c(c4)oc4ccccc45)cc2)cccc13]

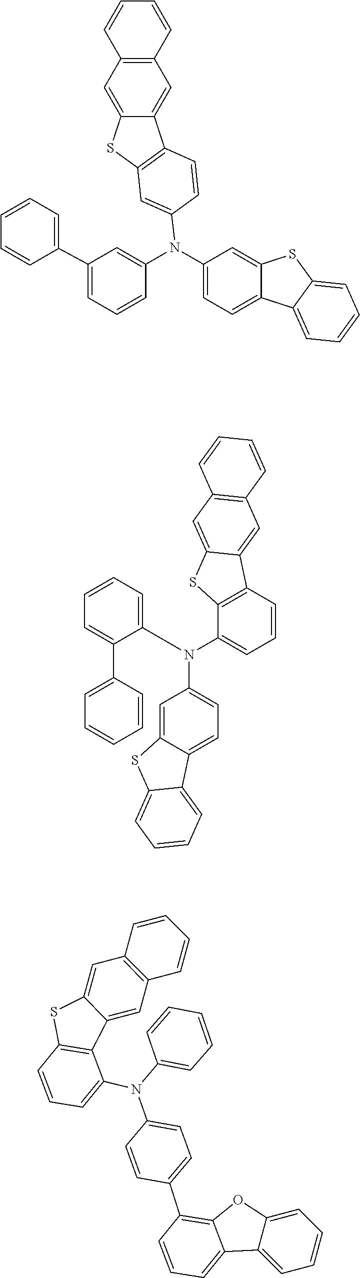 [SMILES: c1ccc(-c2cccc(N(c3ccc4c(c3)sc3ccccc34)c3ccc4c(c3)sc3cc5ccccc5cc34)c2)cc1 ; c1ccc(-c2ccccc2N(c2ccc3c(c2)sc2ccccc23)c2cccc3c2sc2cc4ccccc4cc23)cc1 ; c1ccc(N(c2ccc(-c3cccc4c3oc3ccccc34)cc2)c2cccc3sc4cc5ccccc5cc4c23)cc1]

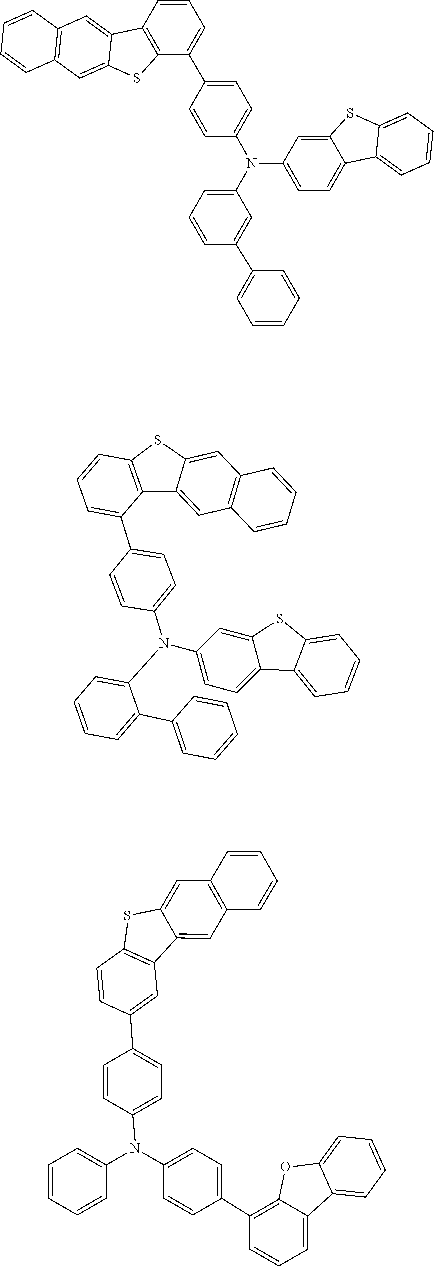 [SMILES: c1ccc(-c2cccc(N(c3ccc(-c4cccc5c4sc4cc6ccccc6cc45)cc3)c3ccc4c(c3)sc3ccccc34)c2)cc1 ; c1ccc(-c2ccccc2N(c2ccc(-c3cccc4sc5cc6ccccc6cc5c34)cc2)c2ccc3c(c2)sc2ccccc23)cc1 ; c1ccc(N(c2ccc(-c3ccc4sc5cc6ccccc6cc5c4c3)cc2)c2ccc(-c3cccc4c3oc3ccccc34)cc2)cc1]

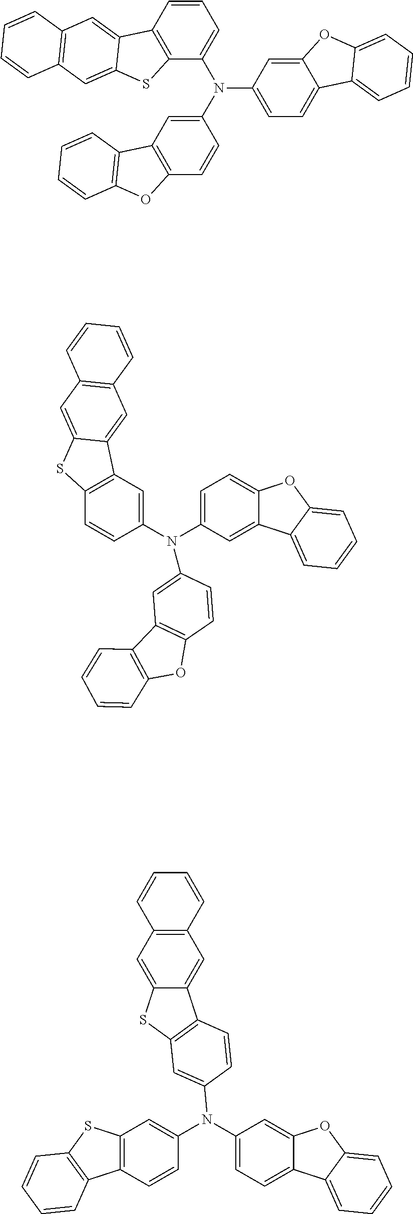 [SMILES: c1ccc2cc3c(cc2c1)sc1c(N(c2ccc4c(c2)oc2ccccc24)c2ccc4oc5ccccc5c4c2)cccc13 ; c1ccc2cc3c(cc2c1)sc1ccc(N(c2ccc4oc5ccccc5c4c2)c2ccc4oc5ccccc5c4c2)cc13 ; c1ccc2cc3c(cc2c1)sc1cc(N(c2ccc4c(c2)oc2ccccc24)c2ccc4c(c2)sc2ccccc24)ccc13]

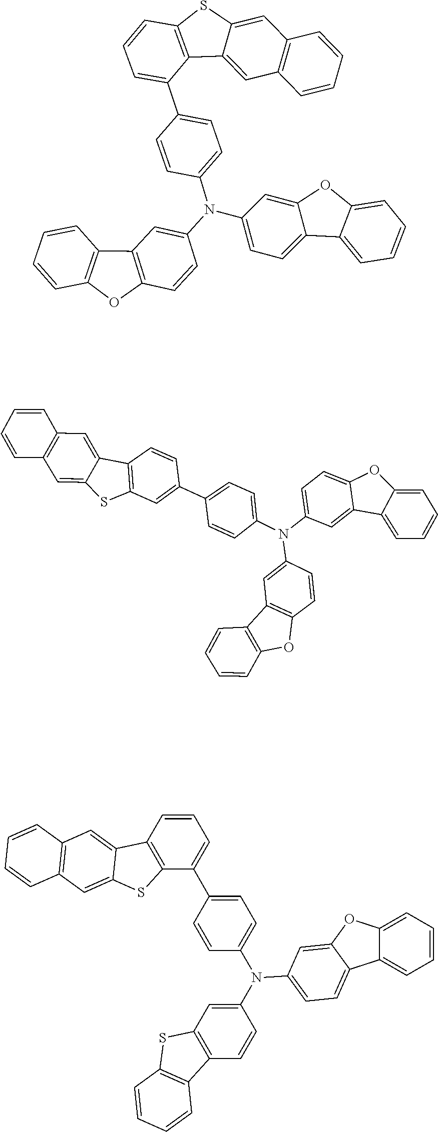 [SMILES: c1ccc2cc3c(cc2c1)sc1cccc(-c2ccc(N(c4ccc5c(c4)oc4ccccc45)c4ccc5oc6ccccc6c5c4)cc2)c13 ; c1ccc2cc3c(cc2c1)sc1cc(-c2ccc(N(c4ccc5oc6ccccc6c5c4)c4ccc5oc6ccccc6c5c4)cc2)ccc13 ; c1ccc2cc3c(cc2c1)sc1c(-c2ccc(N(c4ccc5c(c4)oc4ccccc45)c4ccc5c(c4)sc4ccccc45)cc2)cccc13]

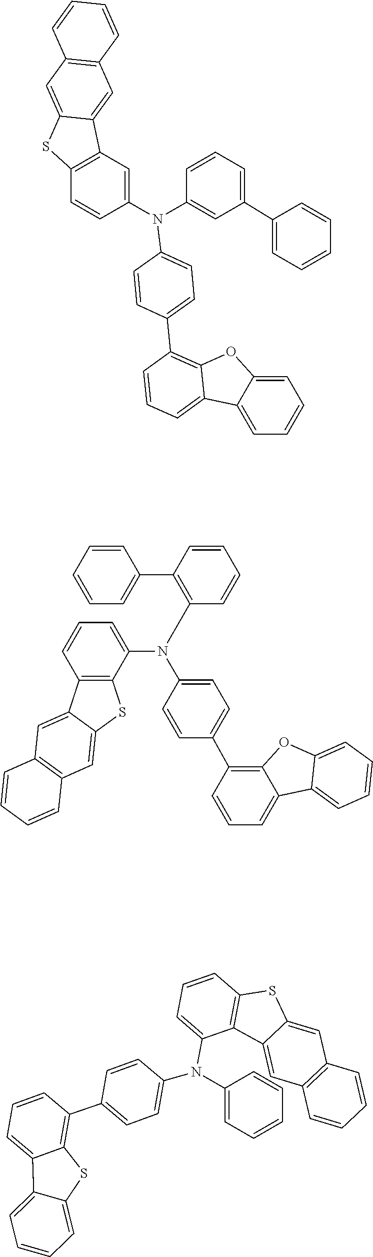 [SMILES: c1ccc(-c2cccc(N(c3ccc(-c4cccc5c4oc4ccccc45)cc3)c3ccc4sc5cc6ccccc6cc5c4c3)c2)cc1 ; c1ccc(-c2ccccc2N(c2ccc(-c3cccc4c3oc3ccccc34)cc2)c2cccc3c2sc2cc4ccccc4cc23)cc1 ; c1ccc(N(c2ccc(-c3cccc4c3sc3ccccc34)cc2)c2cccc3sc4cc5ccccc5cc4c23)cc1]

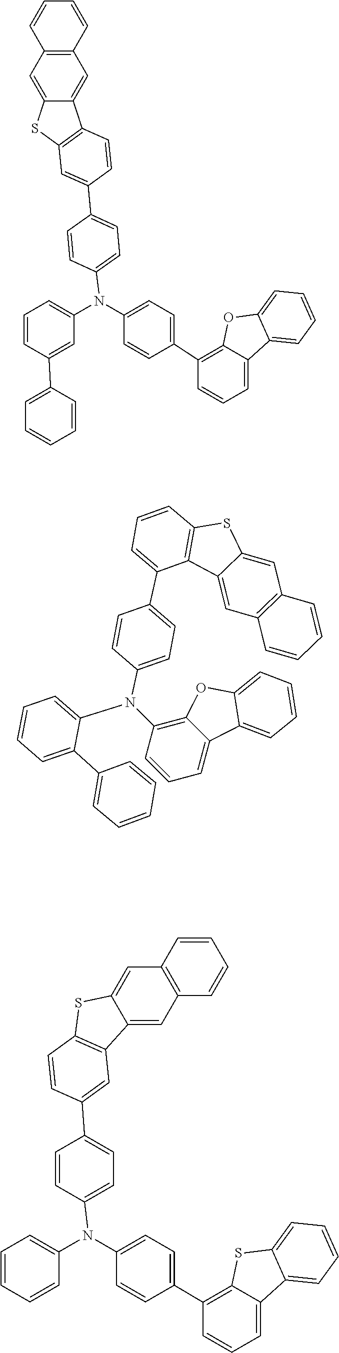 [SMILES: c1ccc(-c2cccc(N(c3ccc(-c4ccc5c(c4)sc4cc6ccccc6cc45)cc3)c3ccc(-c4cccc5c4oc4ccccc45)cc3)c2)cc1 ; c1ccc(-c2ccccc2N(c2ccc(-c3cccc4sc5cc6ccccc6cc5c34)cc2)c2cccc3c2oc2ccccc23)cc1 ; c1ccc(N(c2ccc(-c3ccc4sc5cc6ccccc6cc5c4c3)cc2)c2ccc(-c3cccc4c3sc3ccccc34)cc2)cc1]

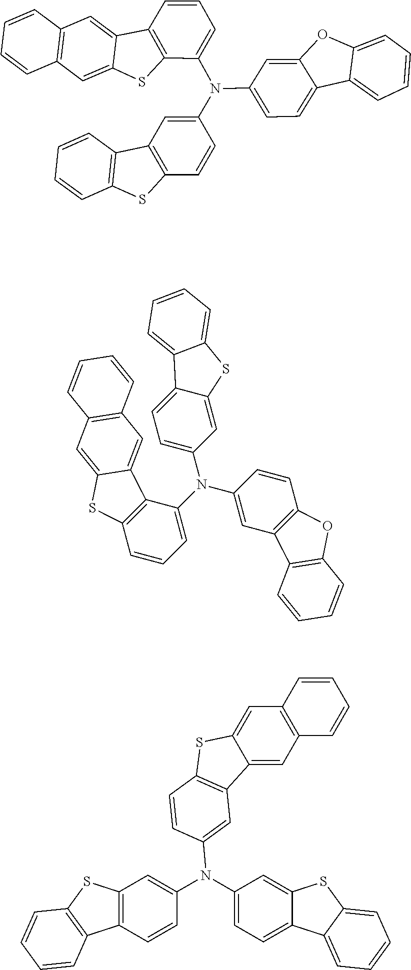 [SMILES: c1ccc2cc3c(cc2c1)sc1c(N(c2ccc4c(c2)oc2ccccc24)c2ccc4sc5ccccc5c4c2)cccc13 ; c1ccc2cc3c(cc2c1)sc1cccc(N(c2ccc4c(c2)sc2ccccc24)c2ccc4oc5ccccc5c4c2)c13 ; c1ccc2cc3c(cc2c1)sc1ccc(N(c2ccc4c(c2)sc2ccccc24)c2ccc4c(c2)sc2ccccc24)cc13]

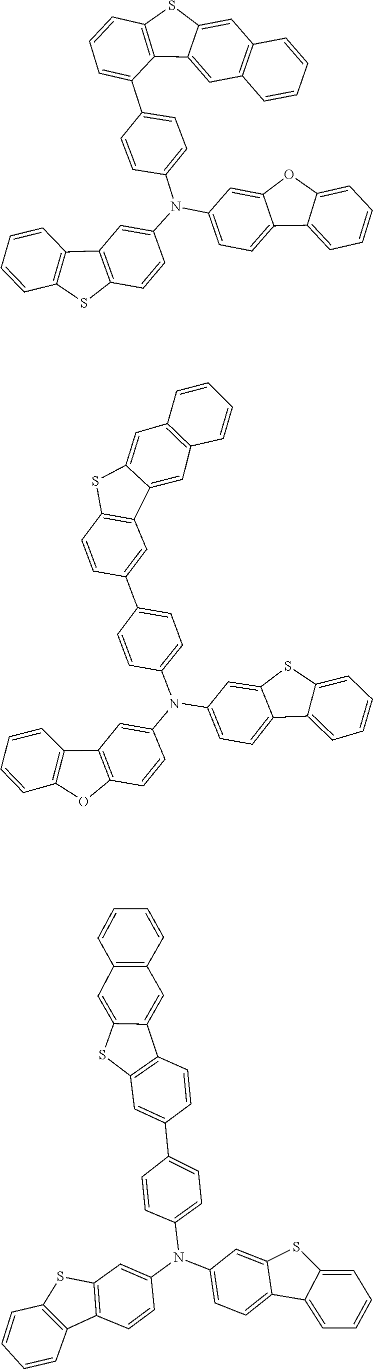 [SMILES: c1ccc2cc3c(cc2c1)sc1cccc(-c2ccc(N(c4ccc5c(c4)oc4ccccc45)c4ccc5sc6ccccc6c5c4)cc2)c13 ; c1ccc2cc3c(cc2c1)sc1ccc(-c2ccc(N(c4ccc5c(c4)sc4ccccc45)c4ccc5oc6ccccc6c5c4)cc2)cc13 ; c1ccc2cc3c(cc2c1)sc1cc(-c2ccc(N(c4ccc5c(c4)sc4ccccc45)c4ccc5c(c4)sc4ccccc45)cc2)ccc13]

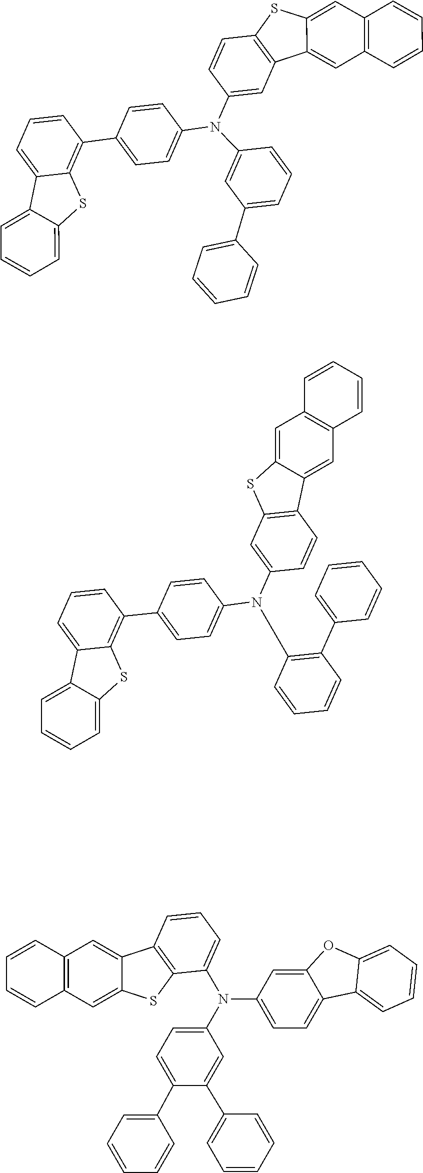 [SMILES: c1ccc(-c2cccc(N(c3ccc(-c4cccc5c4sc4ccccc45)cc3)c3ccc4sc5cc6ccccc6cc5c4c3)c2)cc1 ; c1ccc(-c2ccccc2N(c2ccc(-c3cccc4c3sc3ccccc34)cc2)c2ccc3c(c2)sc2cc4ccccc4cc23)cc1 ; c1ccc(-c2ccc(N(c3ccc4c(c3)oc3ccccc34)c3cccc4c3sc3cc5ccccc5cc34)cc2-c2ccccc2)cc1]

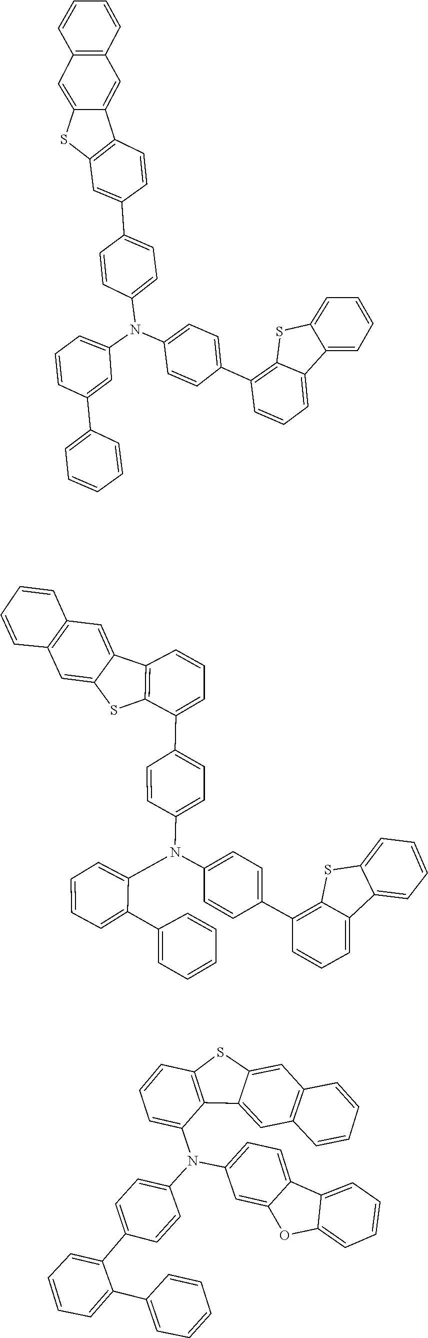 [SMILES: c1ccc(-c2cccc(N(c3ccc(-c4ccc5c(c4)sc4cc6ccccc6cc45)cc3)c3ccc(-c4cccc5c4sc4ccccc45)cc3)c2)cc1 ; c1ccc(-c2ccccc2N(c2ccc(-c3cccc4c3sc3ccccc34)cc2)c2ccc(-c3cccc4c3sc3cc5ccccc5cc34)cc2)cc1 ; c1ccc(-c2ccccc2-c2ccc(N(c3ccc4c(c3)oc3ccccc34)c3cccc4sc5cc6ccccc6cc5c34)cc2)cc1]

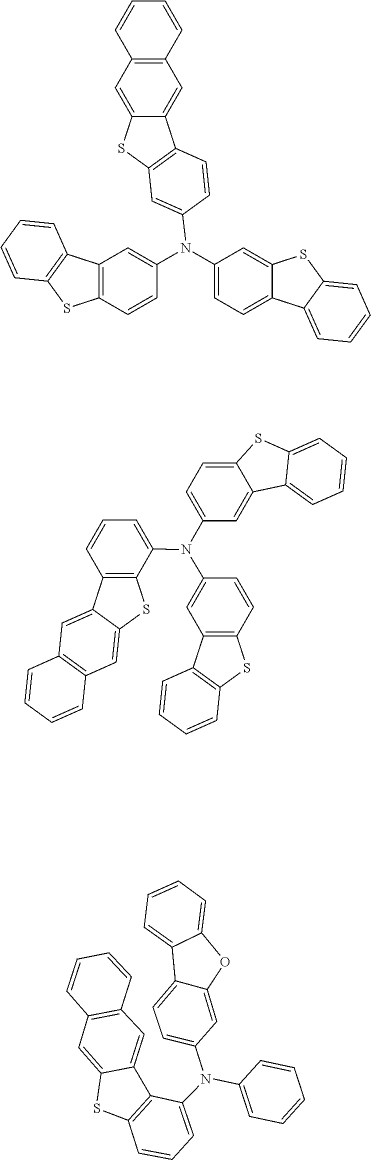 [SMILES: c1ccc2cc3c(cc2c1)sc1cc(N(c2ccc4c(c2)sc2ccccc24)c2ccc4sc5ccccc5c4c2)ccc13 ; c1ccc2cc3c(cc2c1)sc1c(N(c2ccc4sc5ccccc5c4c2)c2ccc4sc5ccccc5c4c2)cccc13 ; c1ccc(N(c2ccc3c(c2)oc2ccccc23)c2cccc3sc4cc5ccccc5cc4c23)cc1]

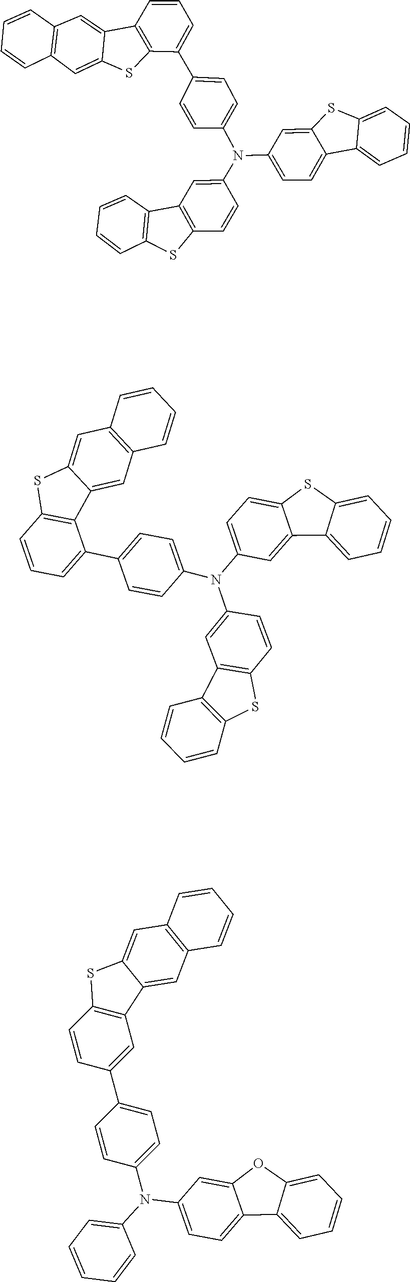 [SMILES: c1ccc2cc3c(cc2c1)sc1c(-c2ccc(N(c4ccc5c(c4)sc4ccccc45)c4ccc5sc6ccccc6c5c4)cc2)cccc13 ; c1ccc2cc3c(cc2c1)sc1cccc(-c2ccc(N(c4ccc5sc6ccccc6c5c4)c4ccc5sc6ccccc6c5c4)cc2)c13 ; c1ccc(N(c2ccc(-c3ccc4sc5cc6ccccc6cc5c4c3)cc2)c2ccc3c(c2)oc2ccccc23)cc1]

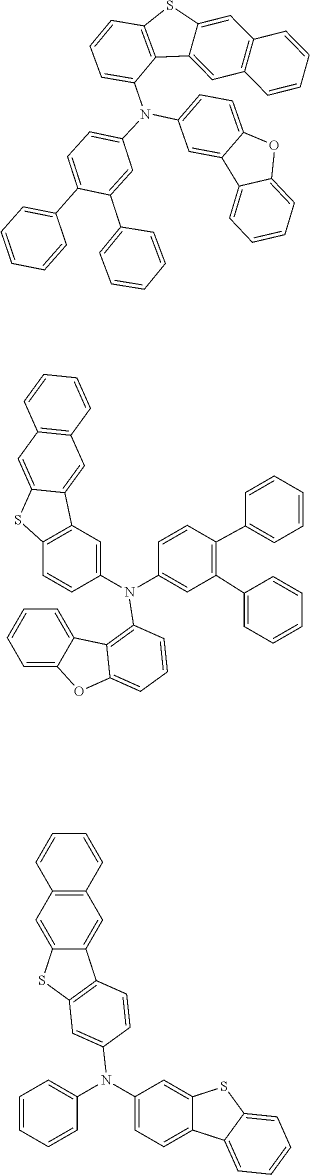 [SMILES: c1ccc(-c2ccc(N(c3ccc4oc5ccccc5c4c3)c3cccc4sc5cc6ccccc6cc5c34)cc2-c2ccccc2)cc1 ; c1ccc(-c2ccc(N(c3ccc4sc5cc6ccccc6cc5c4c3)c3cccc4oc5ccccc5c34)cc2-c2ccccc2)cc1 ; c1ccc(N(c2ccc3c(c2)sc2ccccc23)c2ccc3c(c2)sc2cc4ccccc4cc23)cc1]

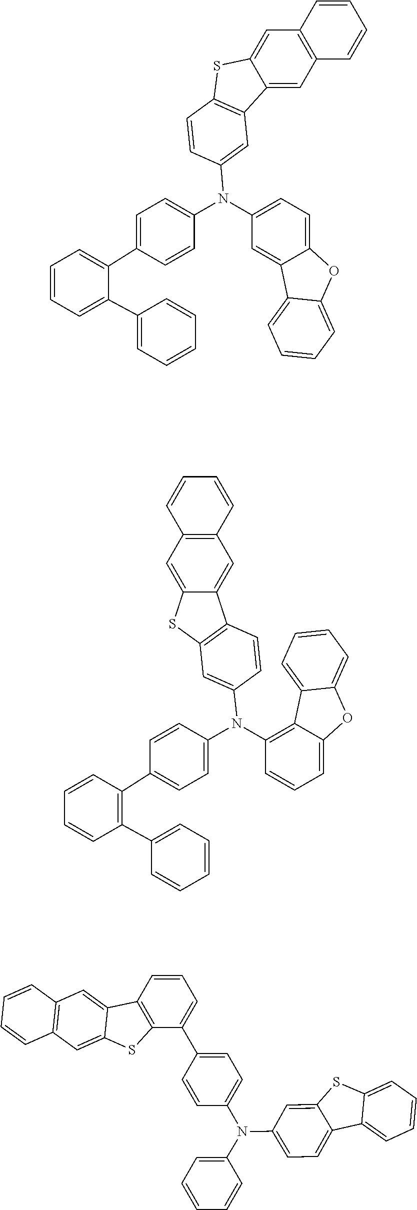 [SMILES: c1ccc(-c2ccccc2-c2ccc(N(c3ccc4oc5ccccc5c4c3)c3ccc4sc5cc6ccccc6cc5c4c3)cc2)cc1 ; c1ccc(-c2ccccc2-c2ccc(N(c3ccc4c(c3)sc3cc5ccccc5cc34)c3cccc4oc5ccccc5c34)cc2)cc1 ; c1ccc(N(c2ccc(-c3cccc4c3sc3cc5ccccc5cc34)cc2)c2ccc3c(c2)sc2ccccc23)cc1]

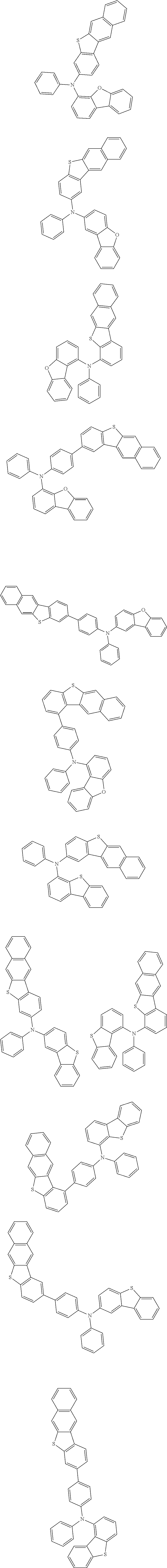

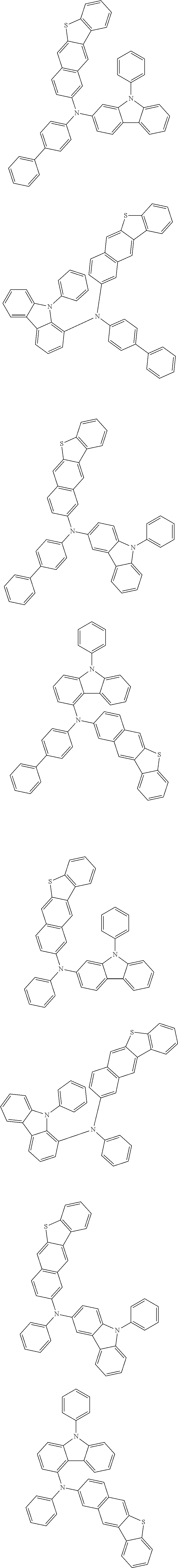

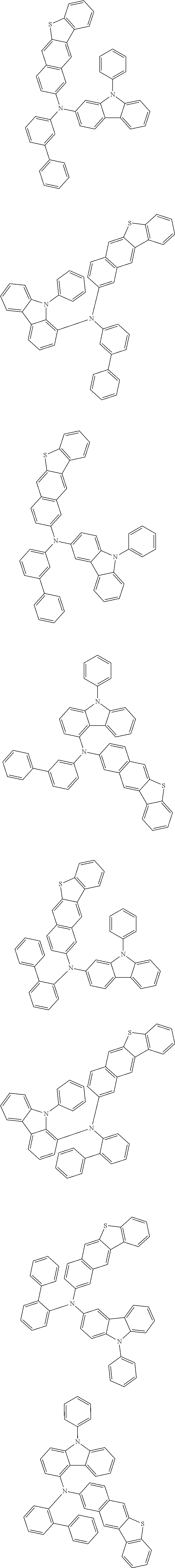

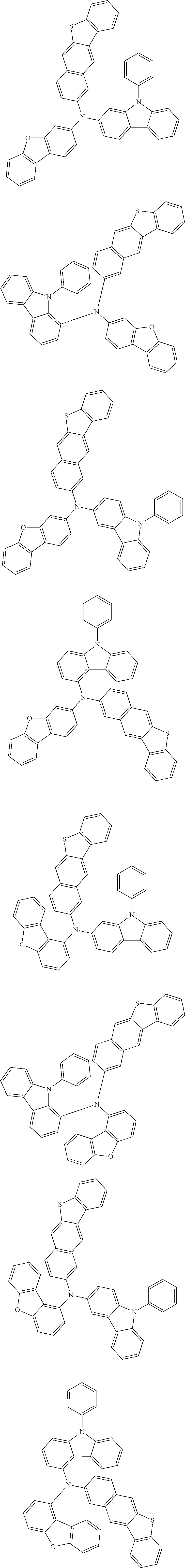

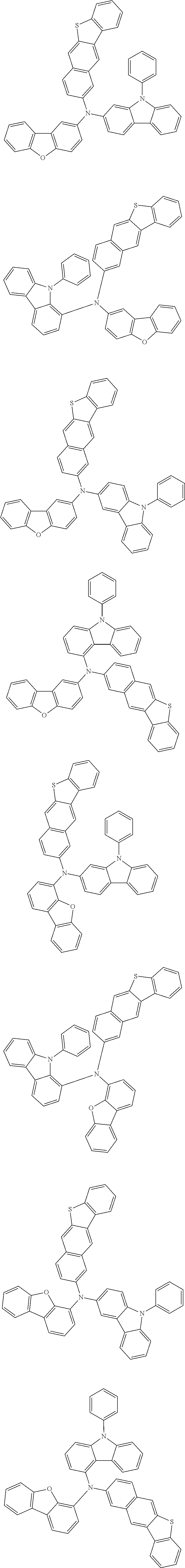

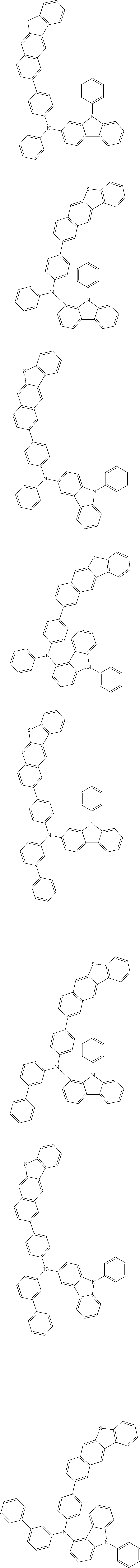

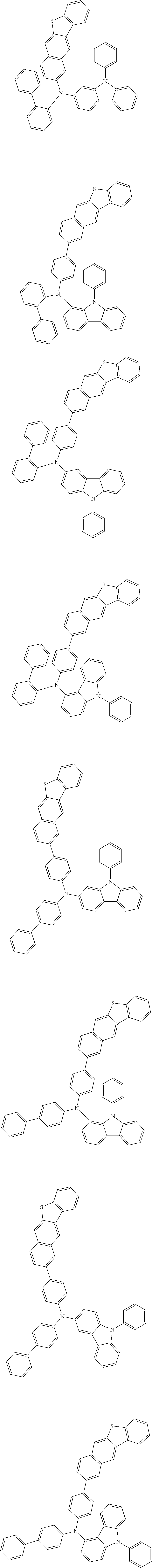

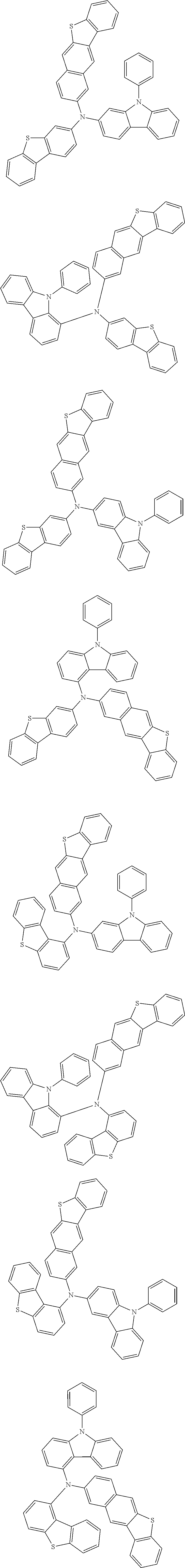

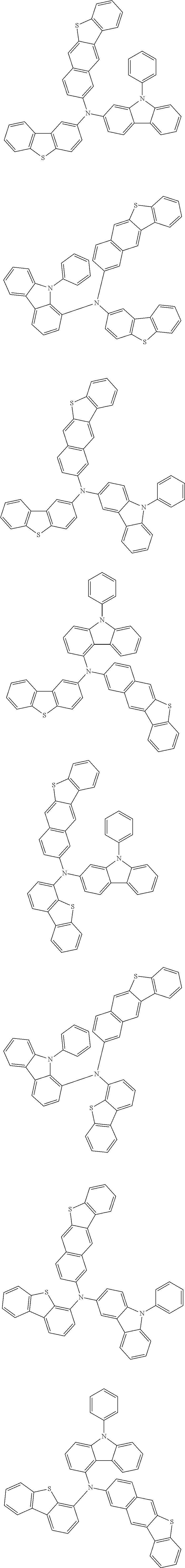

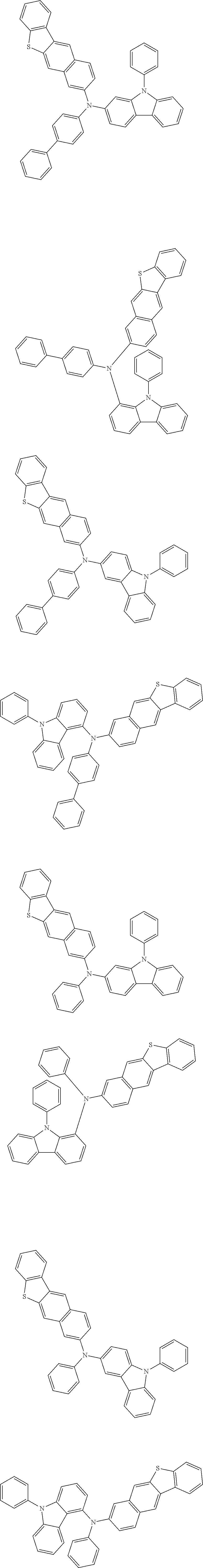

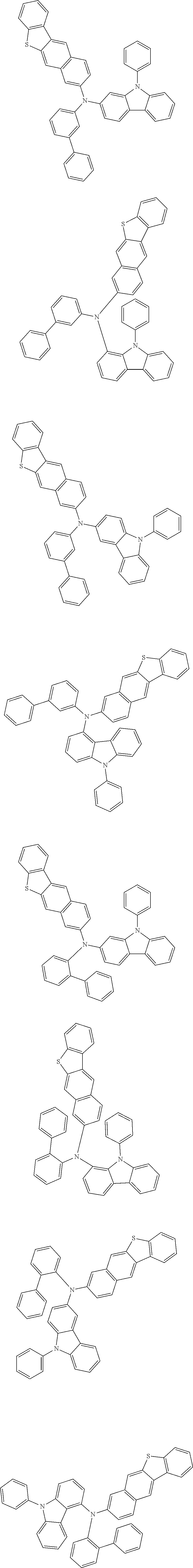

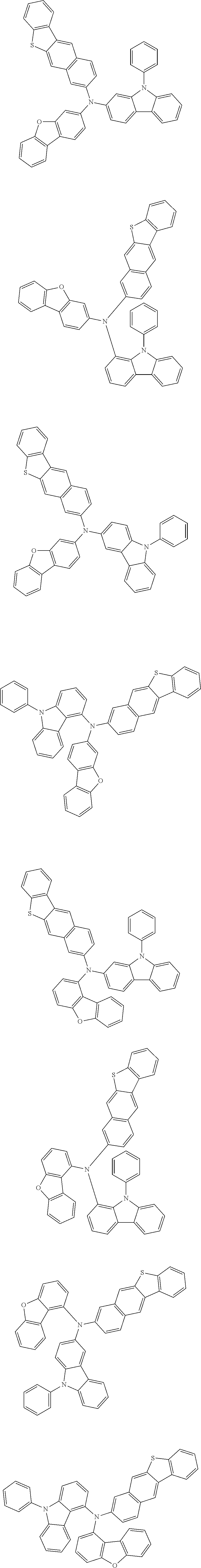

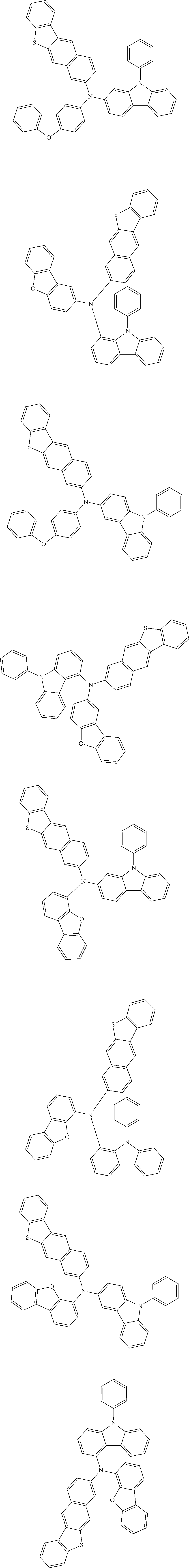

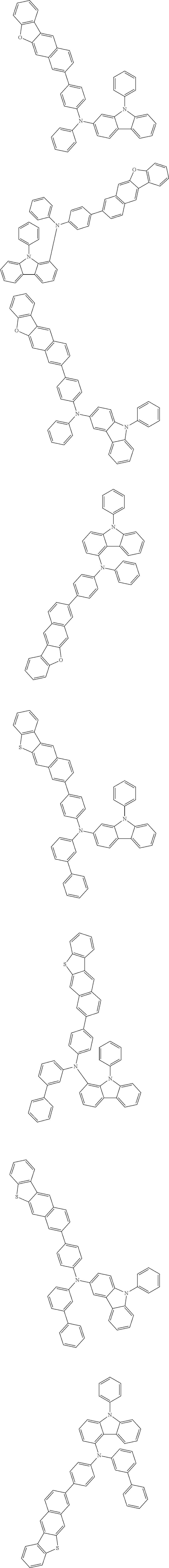

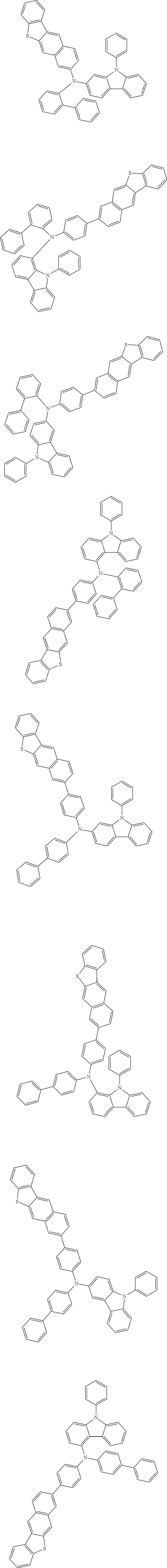

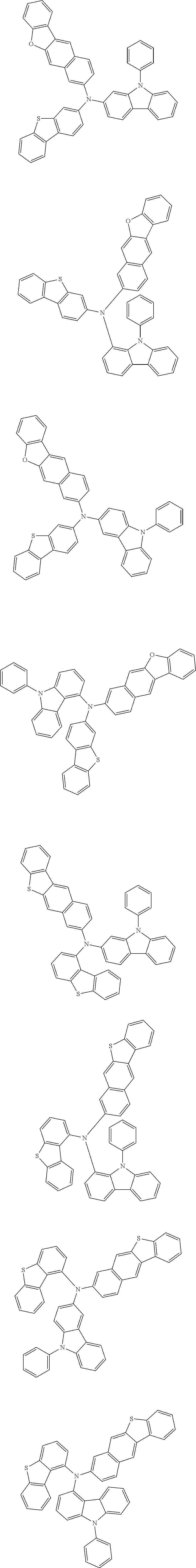

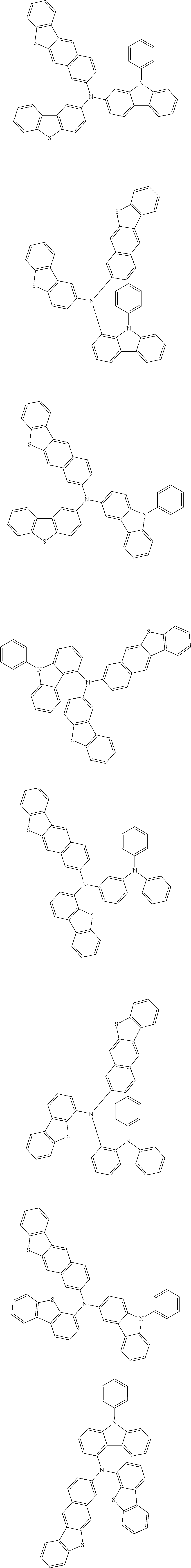

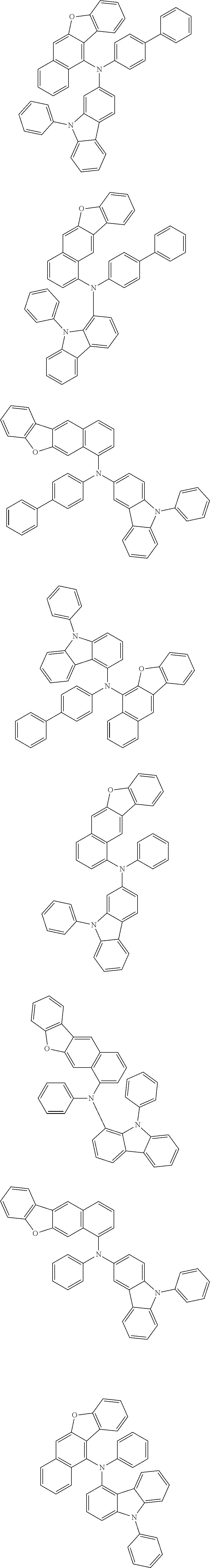

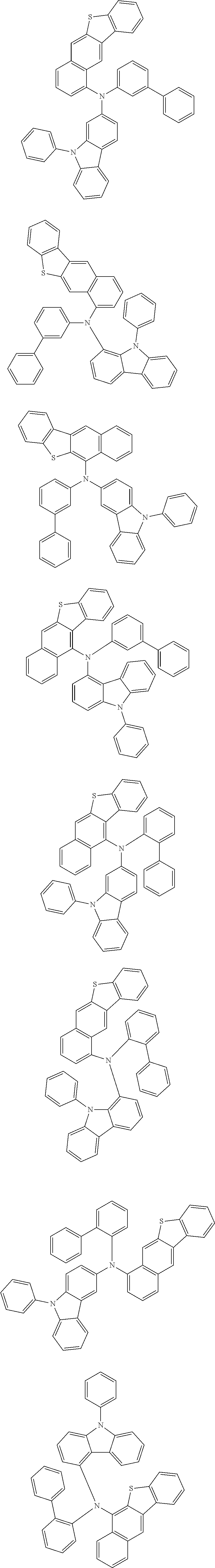

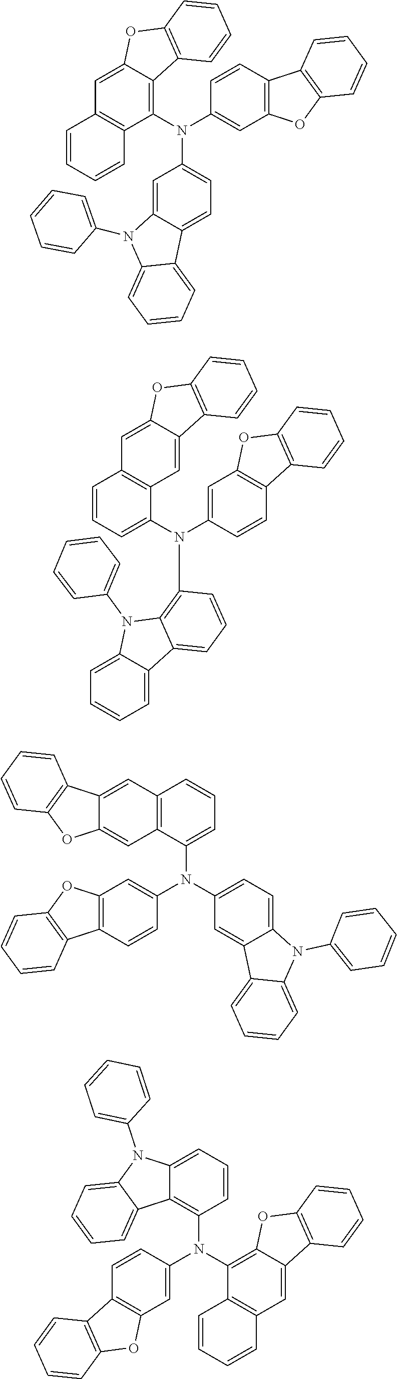

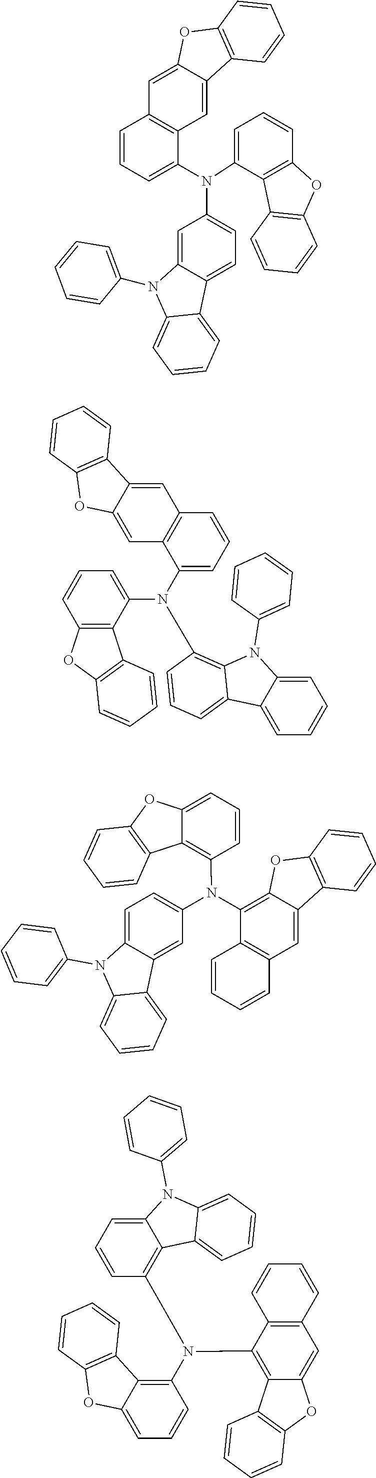

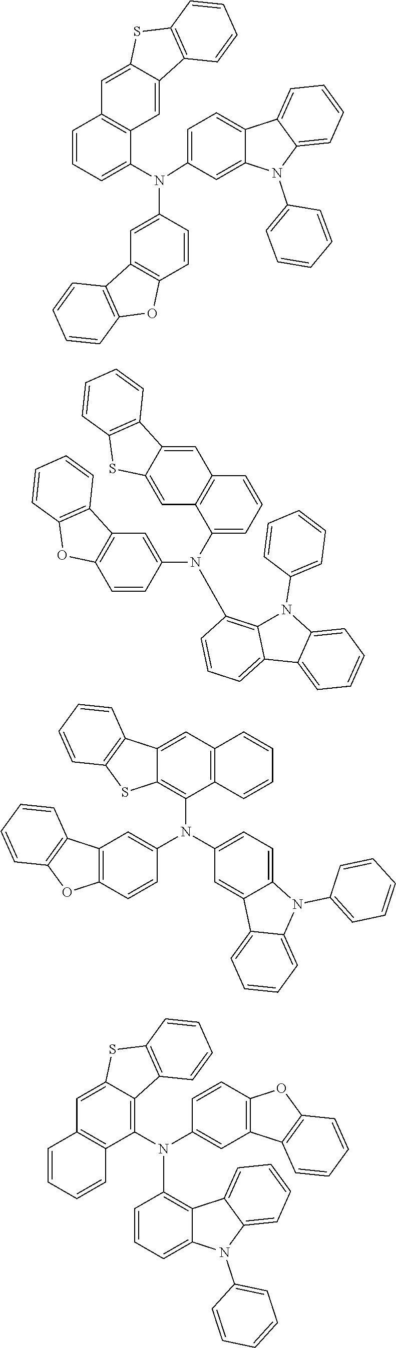

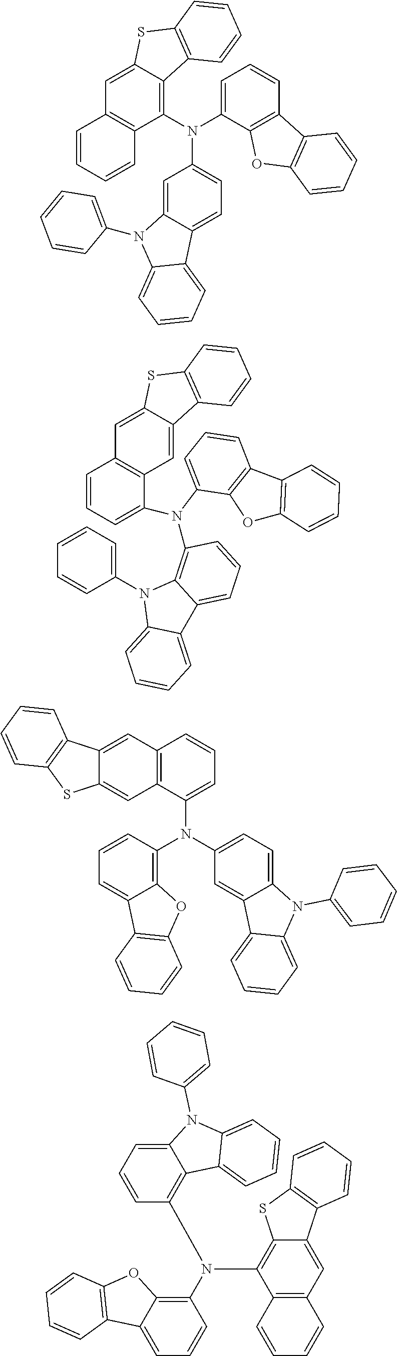

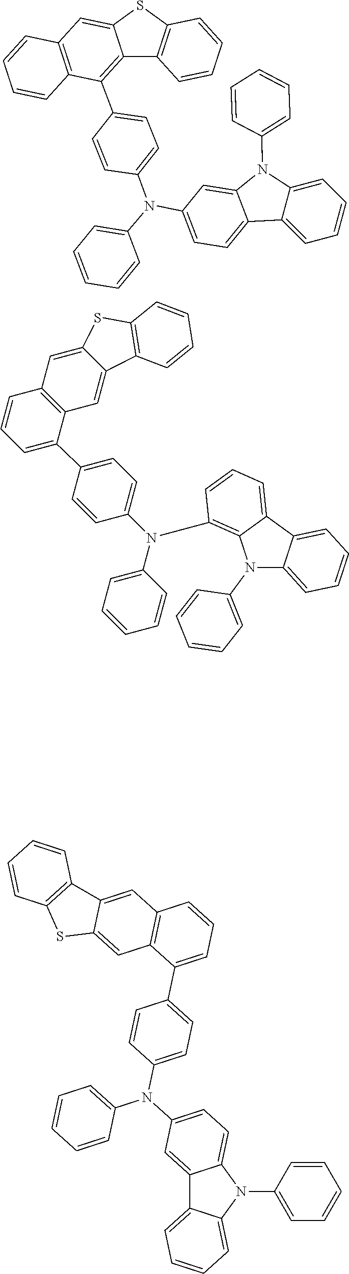

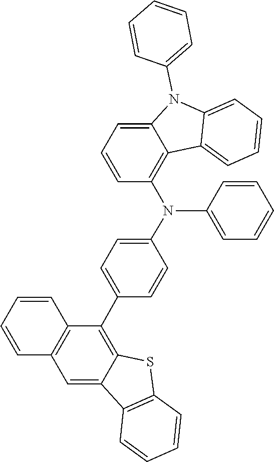 [SMILES: c1ccc(N(c2ccc(-c3c4ccccc4cc4c3sc3ccccc34)cc2)c2cccc3c2c2ccccc2n3-c2ccccc2)cc1]

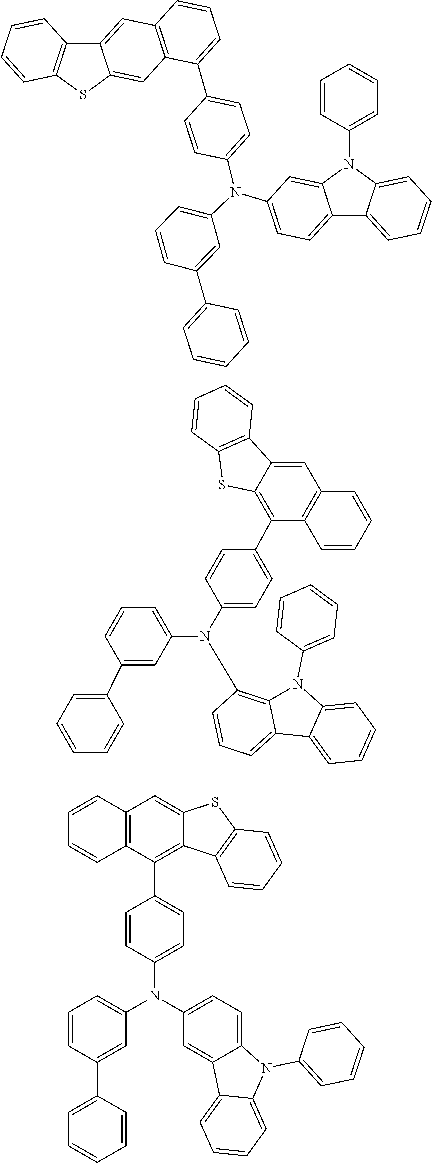

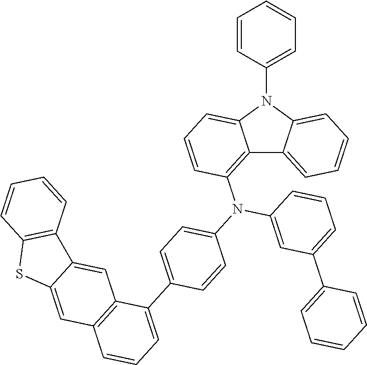 [SMILES: c1ccc(-c2cccc(N(c3ccc(-c4cccc5cc6sc7ccccc7c6cc45)cc3)c3cccc4c3c3ccccc3n4-c3ccccc3)c2)cc1]

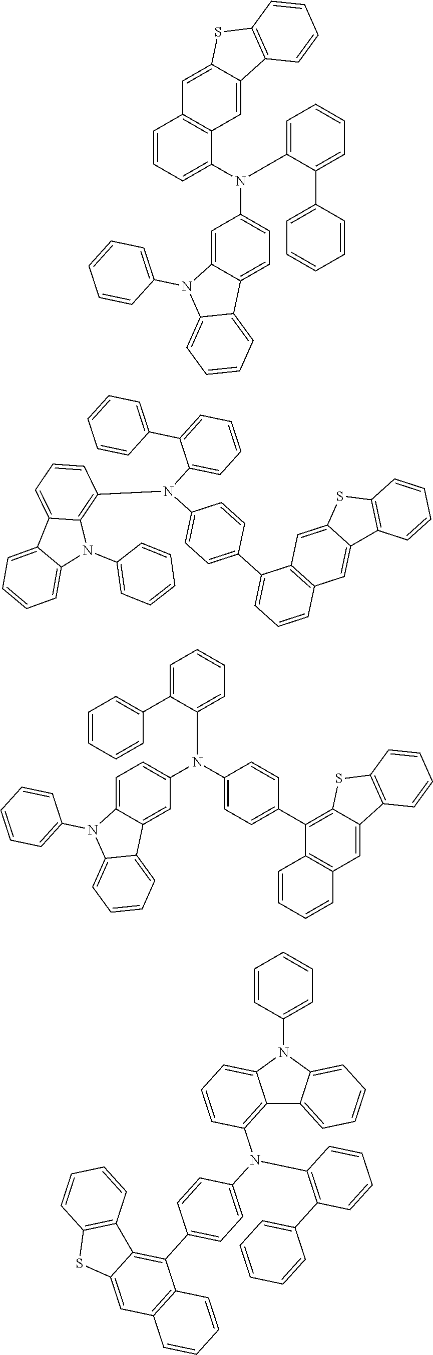

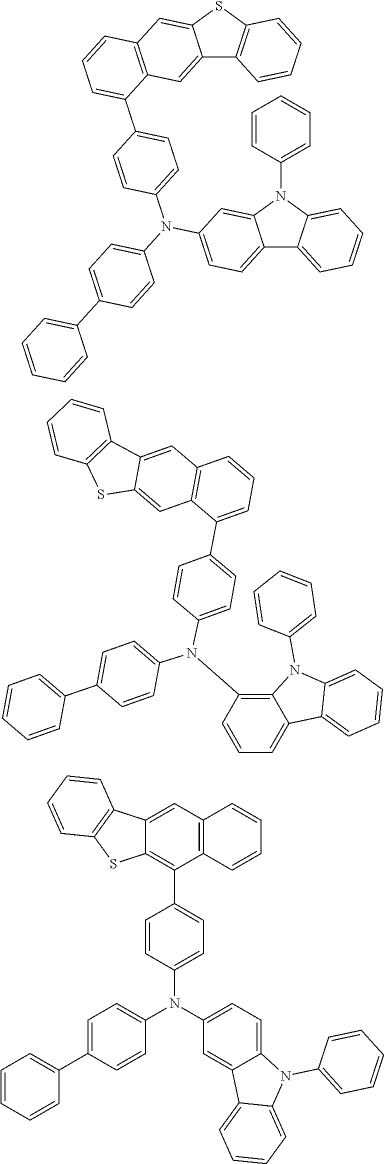

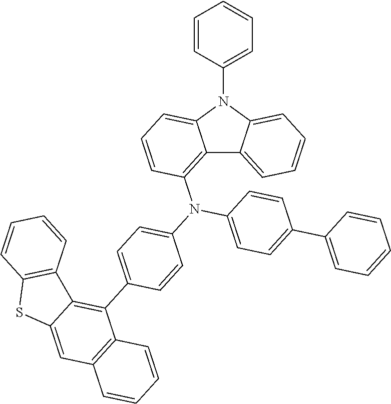 [SMILES: c1ccc(-c2ccc(N(c3ccc(-c4c5ccccc5cc5sc6ccccc6c45)cc3)c3cccc4c3c3ccccc3n4-c3ccccc3)cc2)cc1]

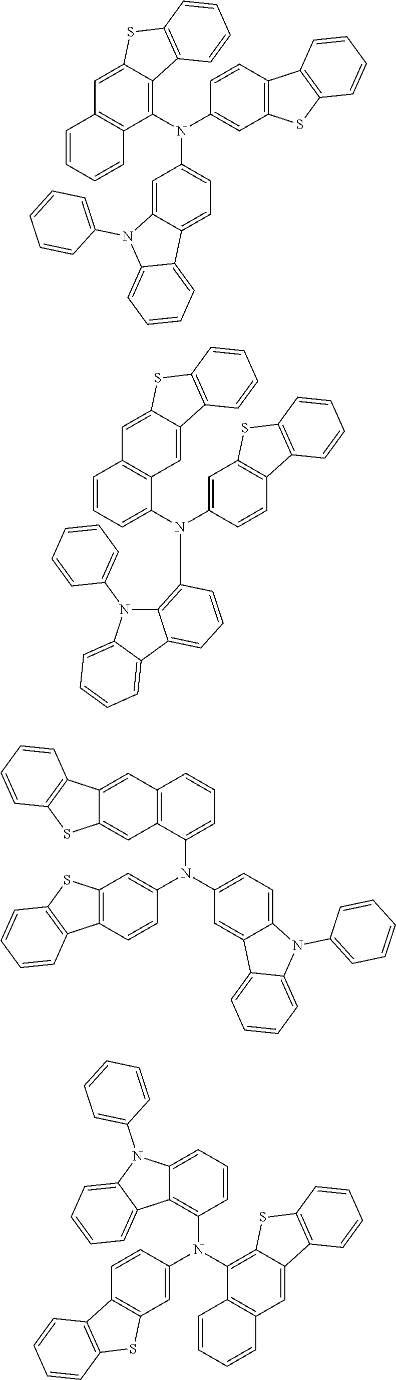

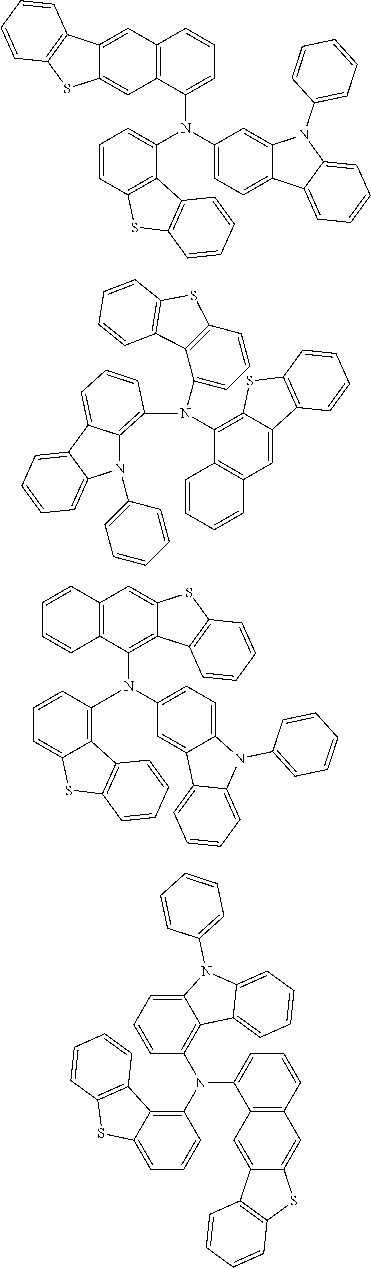

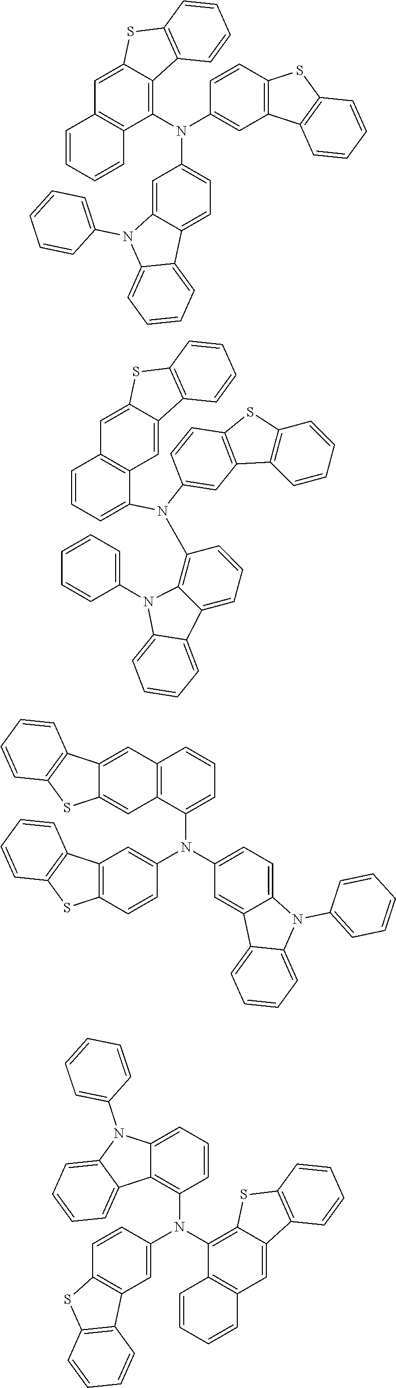

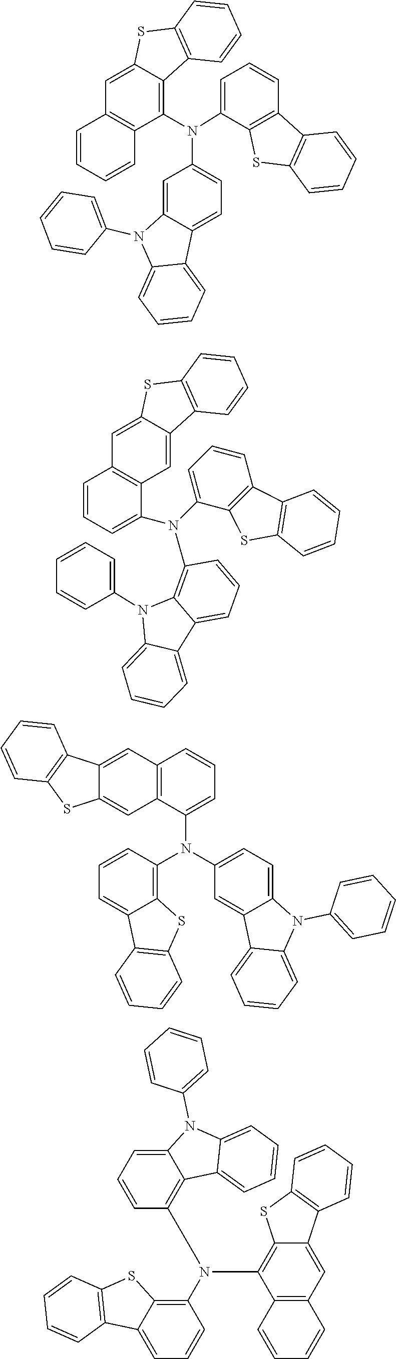

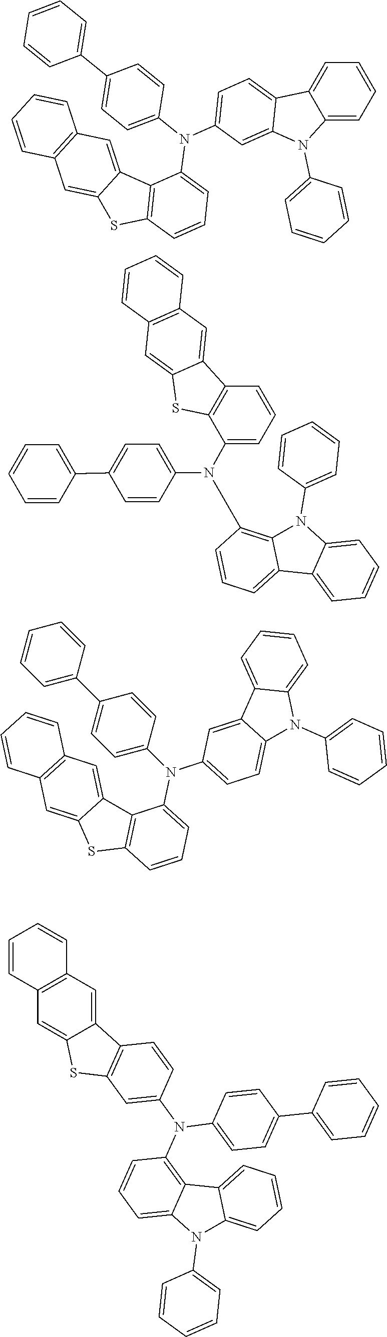

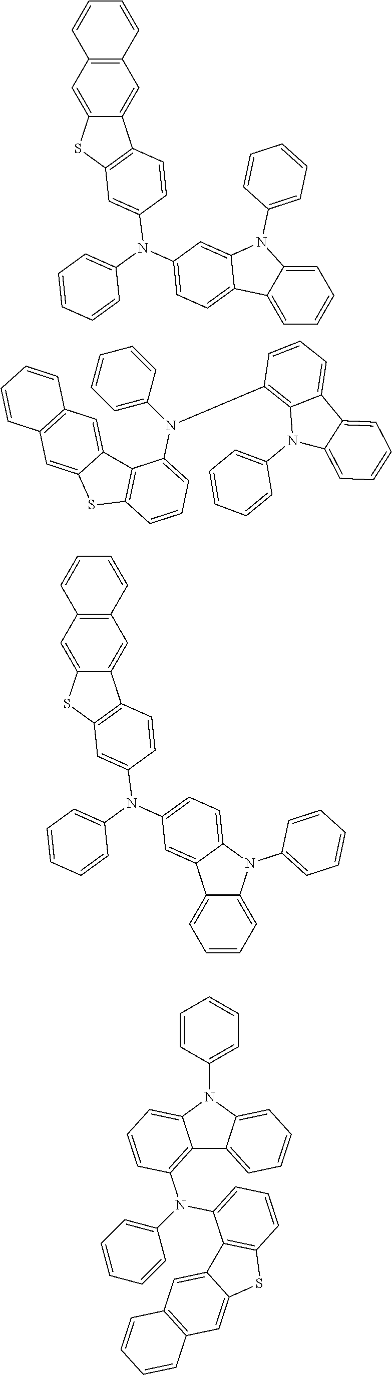

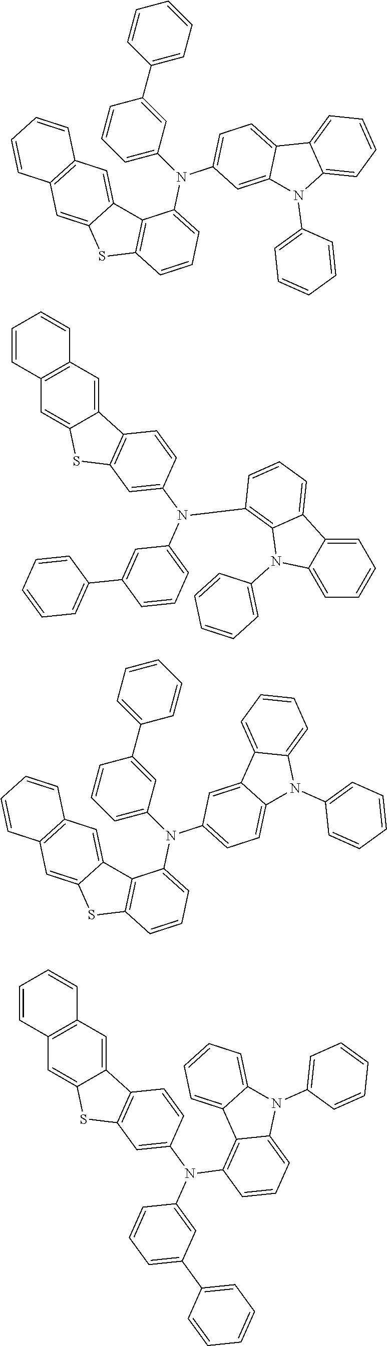 [SMILES: c1ccc(-c2cccc(N(c3ccc4c5ccccc5n(-c5ccccc5)c4c3)c3cccc4sc5cc6ccccc6cc5c34)c2)cc1 ; c1ccc(-c2cccc(N(c3ccc4c(c3)sc3cc5ccccc5cc34)c3cccc4c5ccccc5n(-c5ccccc5)c34)c2)cc1 ; c1ccc(-c2cccc(N(c3ccc4c(c3)c3ccccc3n4-c3ccccc3)c3cccc4sc5cc6ccccc6cc5c34)c2)cc1 ; c1ccc(-c2cccc(N(c3ccc4c(c3)sc3cc5ccccc5cc34)c3cccc4c3c3ccccc3n4-c3ccccc3)c2)cc1]

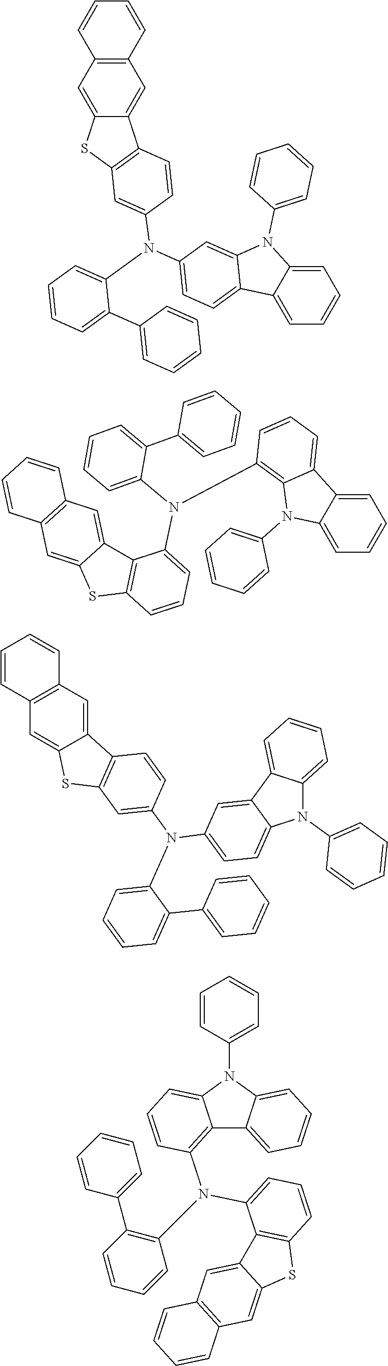

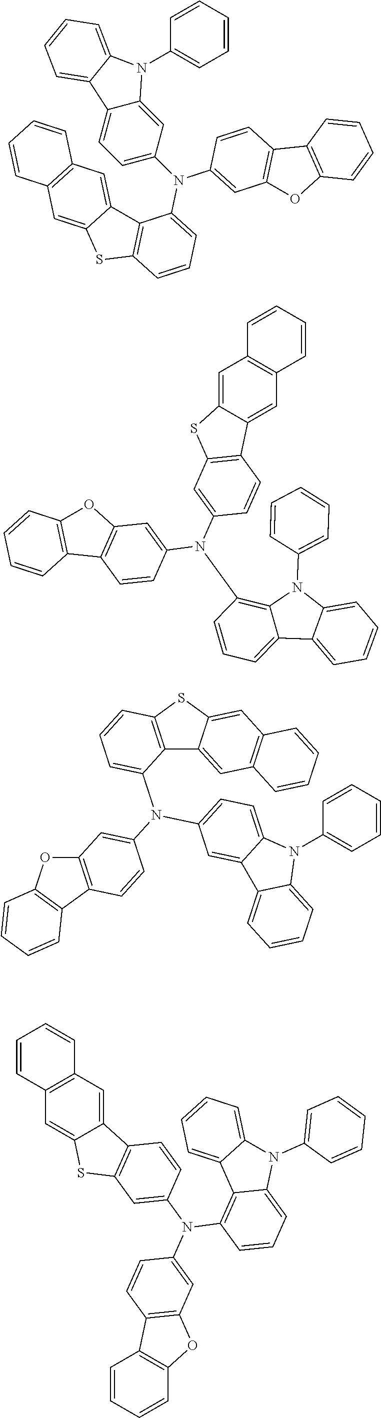 [SMILES: c1ccc(-n2c3ccccc3c3ccc(N(c4ccc5c(c4)oc4ccccc45)c4cccc5sc6cc7ccccc7cc6c45)cc32)cc1 ; c1ccc(-n2c3ccccc3c3cccc(N(c4ccc5c(c4)oc4ccccc45)c4ccc5c(c4)sc4cc6ccccc6cc45)c32)cc1 ; c1ccc(-n2c3ccccc3c3cc(N(c4ccc5c(c4)oc4ccccc45)c4cccc5sc6cc7ccccc7cc6c45)ccc32)cc1 ; c1ccc(-n2c3ccccc3c3c(N(c4ccc5c(c4)oc4ccccc45)c4ccc5c(c4)sc4cc6ccccc6cc45)cccc32)cc1]

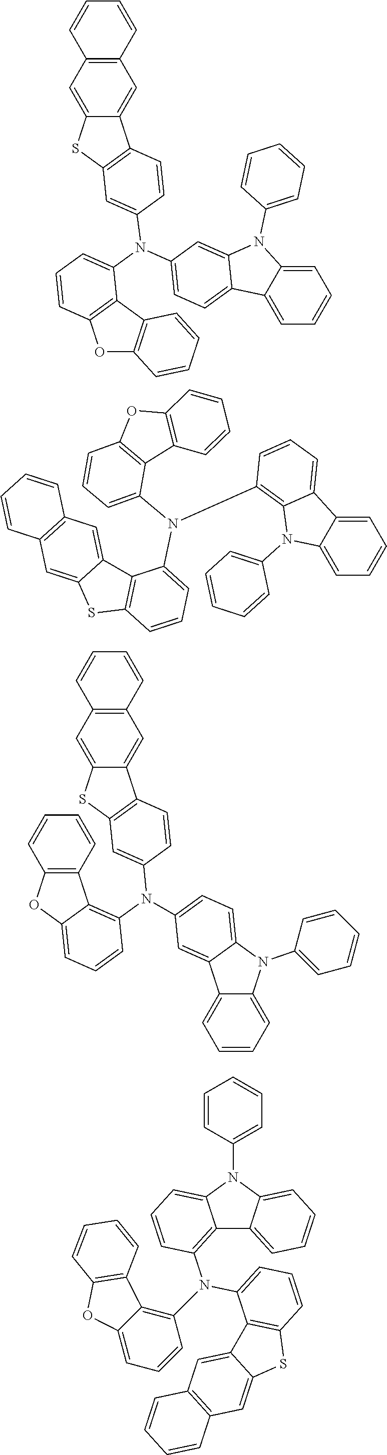

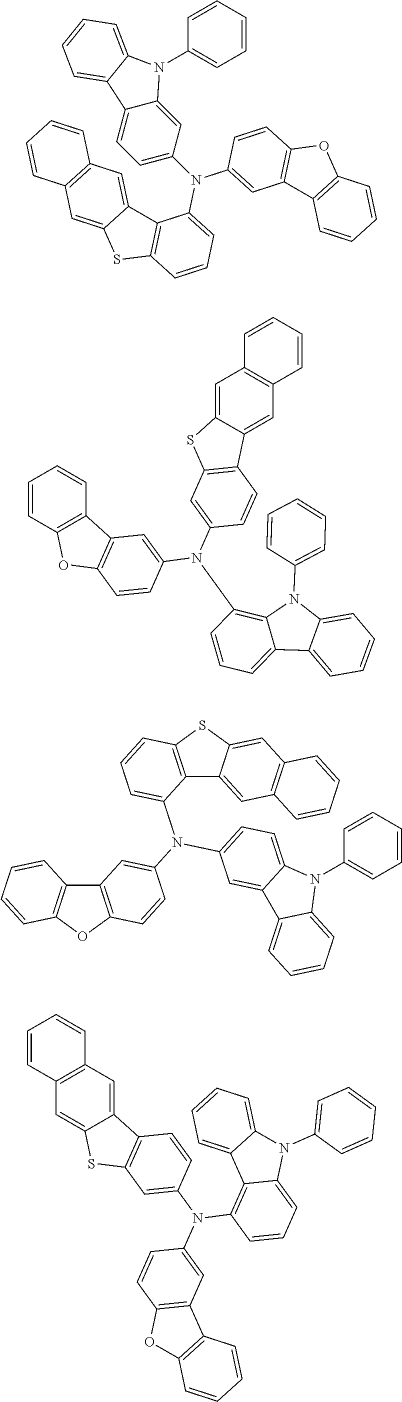 [SMILES: c1ccc(-n2c3ccccc3c3ccc(N(c4ccc5oc6ccccc6c5c4)c4cccc5sc6cc7ccccc7cc6c45)cc32)cc1 ; c1ccc(-n2c3ccccc3c3cccc(N(c4ccc5c(c4)sc4cc6ccccc6cc45)c4ccc5oc6ccccc6c5c4)c32)cc1 ; c1ccc(-n2c3ccccc3c3cc(N(c4ccc5oc6ccccc6c5c4)c4cccc5sc6cc7ccccc7cc6c45)ccc32)cc1 ; c1ccc(-n2c3ccccc3c3c(N(c4ccc5c(c4)sc4cc6ccccc6cc45)c4ccc5oc6ccccc6c5c4)cccc32)cc1]

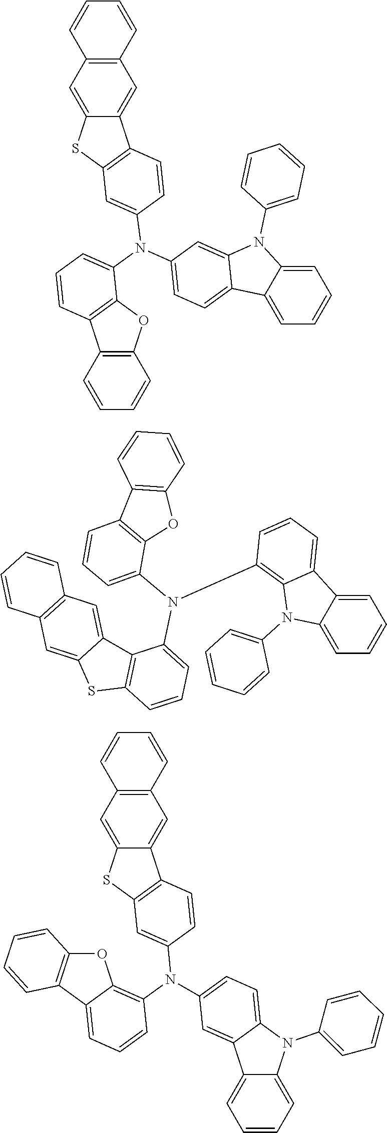

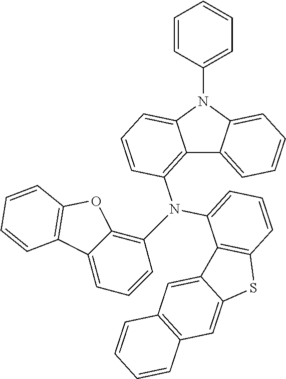 [SMILES: c1ccc(-n2c3ccccc3c3c(N(c4cccc5c4oc4ccccc45)c4cccc5sc6cc7ccccc7cc6c45)cccc32)cc1]

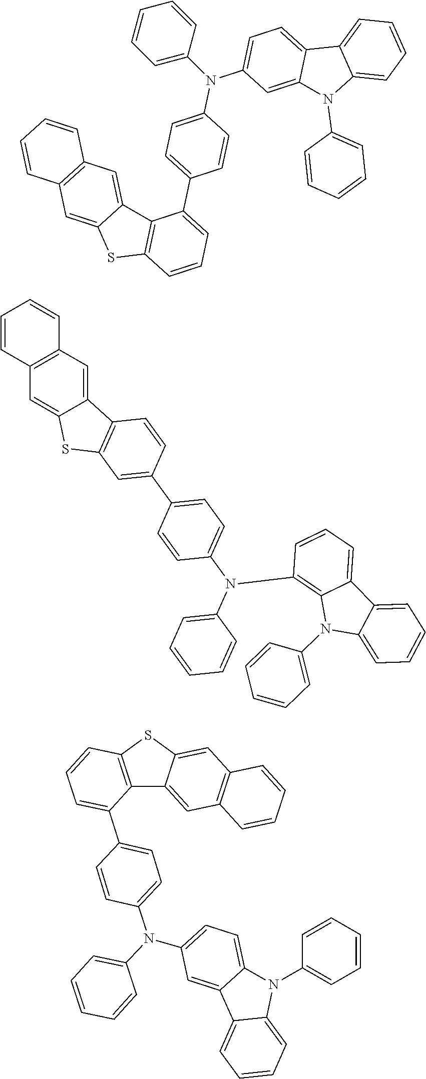 [SMILES: c1ccc(N(c2ccc(-c3cccc4sc5cc6ccccc6cc5c34)cc2)c2ccc3c4ccccc4n(-c4ccccc4)c3c2)cc1 ; c1ccc(N(c2ccc(-c3ccc4c(c3)sc3cc5ccccc5cc34)cc2)c2cccc3c4ccccc4n(-c4ccccc4)c23)cc1 ; c1ccc(N(c2ccc(-c3cccc4sc5cc6ccccc6cc5c34)cc2)c2ccc3c(c2)c2ccccc2n3-c2ccccc2)cc1]

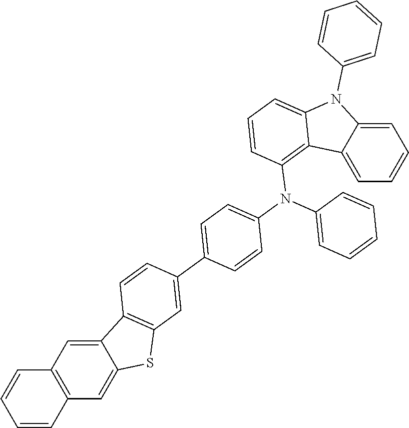 [SMILES: c1ccc(N(c2ccc(-c3ccc4c(c3)sc3cc5ccccc5cc34)cc2)c2cccc3c2c2ccccc2n3-c2ccccc2)cc1]

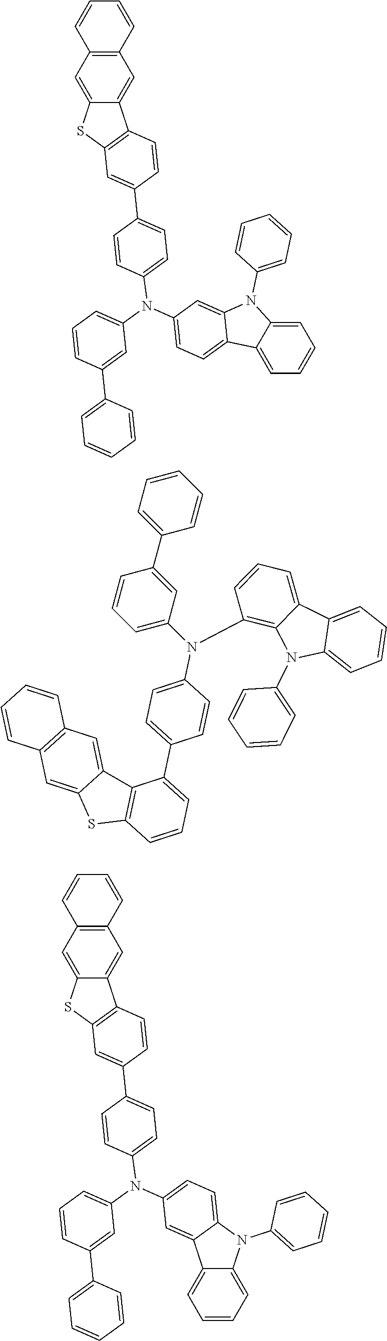

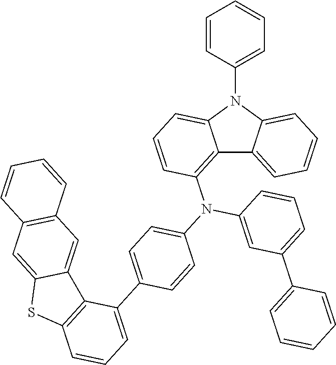 [SMILES: c1ccc(-c2cccc(N(c3ccc(-c4cccc5sc6cc7ccccc7cc6c45)cc3)c3cccc4c3c3ccccc3n4-c3ccccc3)c2)cc1]

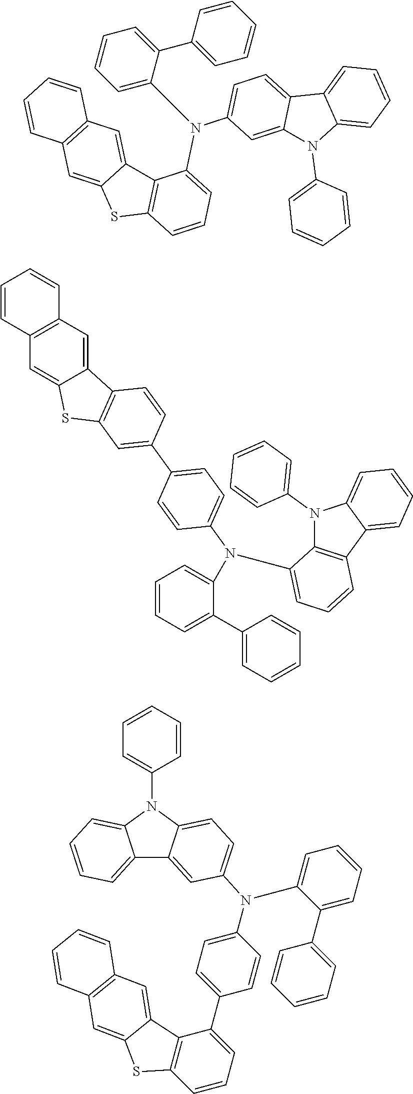 [SMILES: c1ccc(-c2ccccc2N(c2ccc3c4ccccc4n(-c4ccccc4)c3c2)c2cccc3sc4cc5ccccc5cc4c23)cc1 ; c1ccc(-c2ccccc2N(c2ccc(-c3ccc4c(c3)sc3cc5ccccc5cc34)cc2)c2cccc3c4ccccc4n(-c4ccccc4)c23)cc1 ; c1ccc(-c2ccccc2N(c2ccc(-c3cccc4sc5cc6ccccc6cc5c34)cc2)c2ccc3c(c2)c2ccccc2n3-c2ccccc2)cc1]

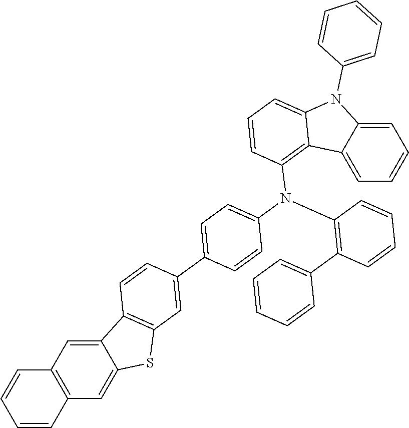 [SMILES: c1ccc(-c2ccccc2N(c2ccc(-c3ccc4c(c3)sc3cc5ccccc5cc34)cc2)c2cccc3c2c2ccccc2n3-c2ccccc2)cc1]

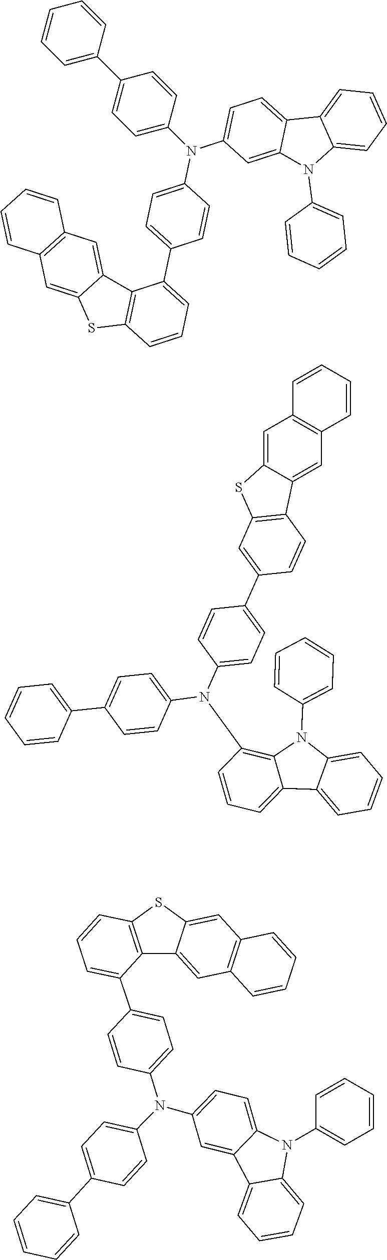 [SMILES: c1ccc(-c2ccc(N(c3ccc(-c4cccc5sc6cc7ccccc7cc6c45)cc3)c3ccc4c5ccccc5n(-c5ccccc5)c4c3)cc2)cc1 ; c1ccc(-c2ccc(N(c3ccc(-c4ccc5c(c4)sc4cc6ccccc6cc45)cc3)c3cccc4c5ccccc5n(-c5ccccc5)c34)cc2)cc1 ; c1ccc(-c2ccc(N(c3ccc(-c4cccc5sc6cc7ccccc7cc6c45)cc3)c3ccc4c(c3)c3ccccc3n4-c3ccccc3)cc2)cc1]

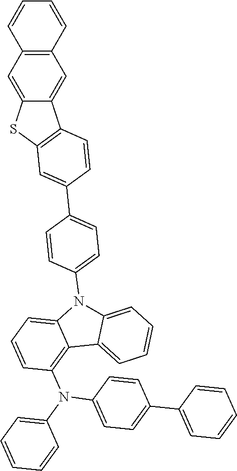 [SMILES: c1ccc(-c2ccc(N(c3ccccc3)c3cccc4c3c3ccccc3n4-c3ccc(-c4ccc5c(c4)sc4cc6ccccc6cc45)cc3)cc2)cc1]

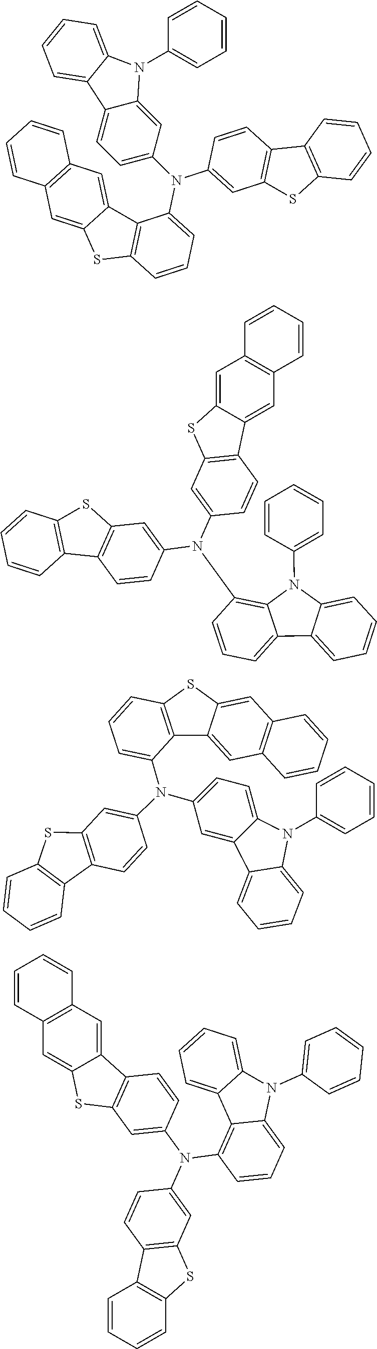

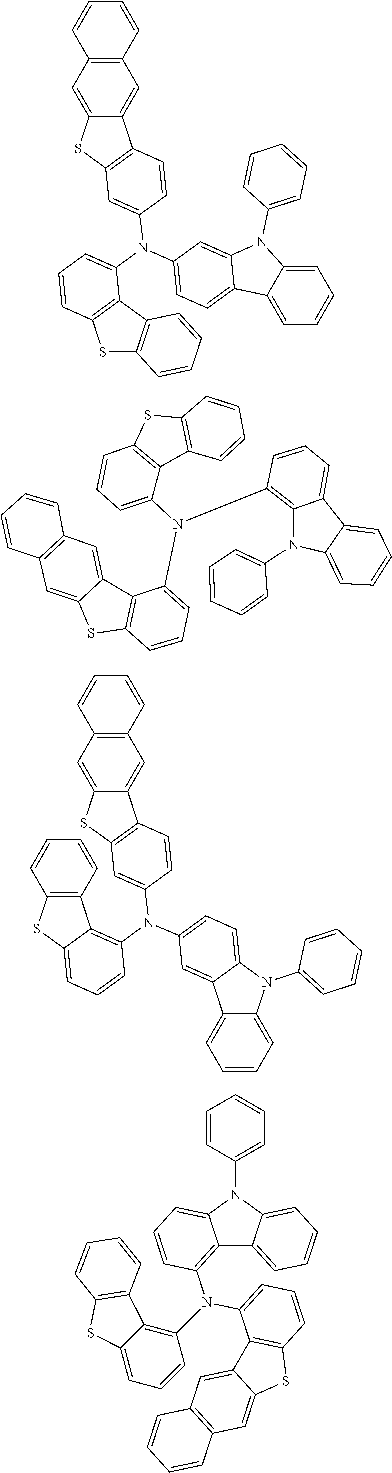

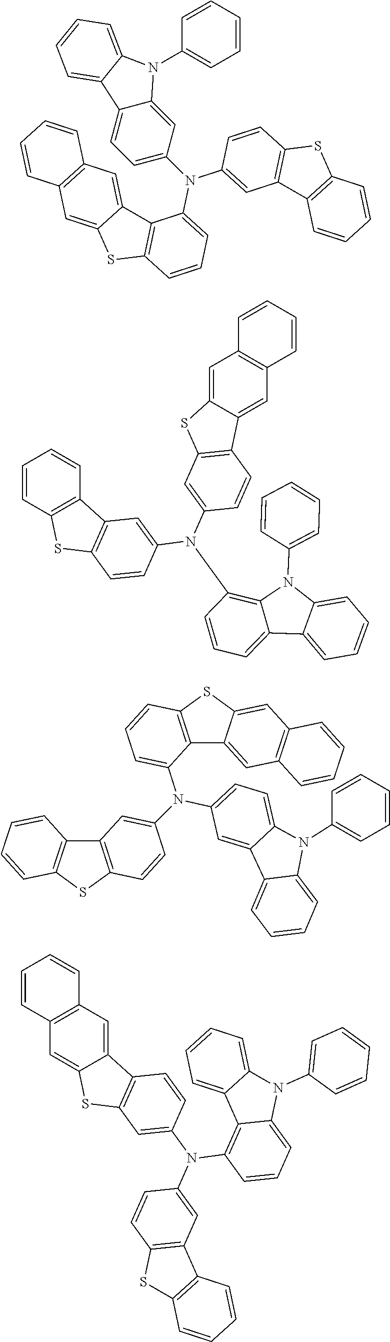

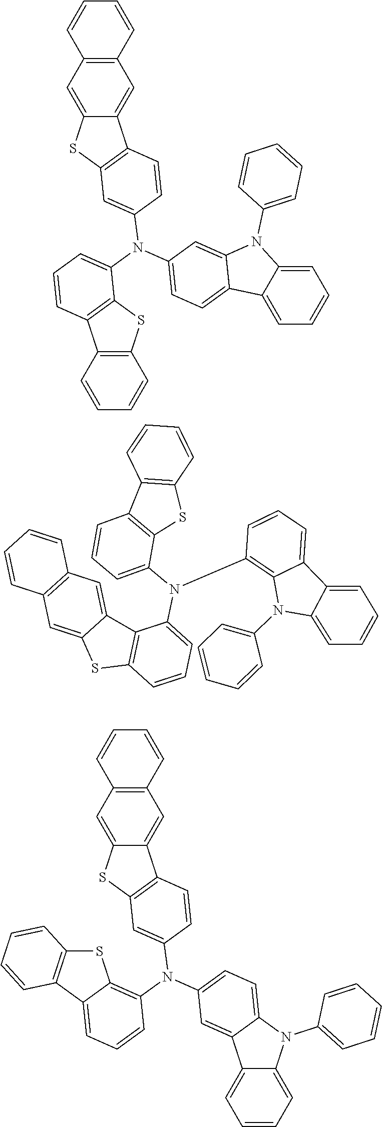

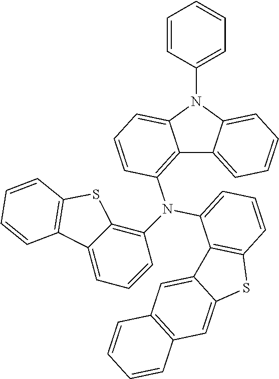 [SMILES: c1ccc(-n2c3ccccc3c3c(N(c4cccc5c4sc4ccccc45)c4cccc5sc6cc7ccccc7cc6c45)cccc32)cc1]

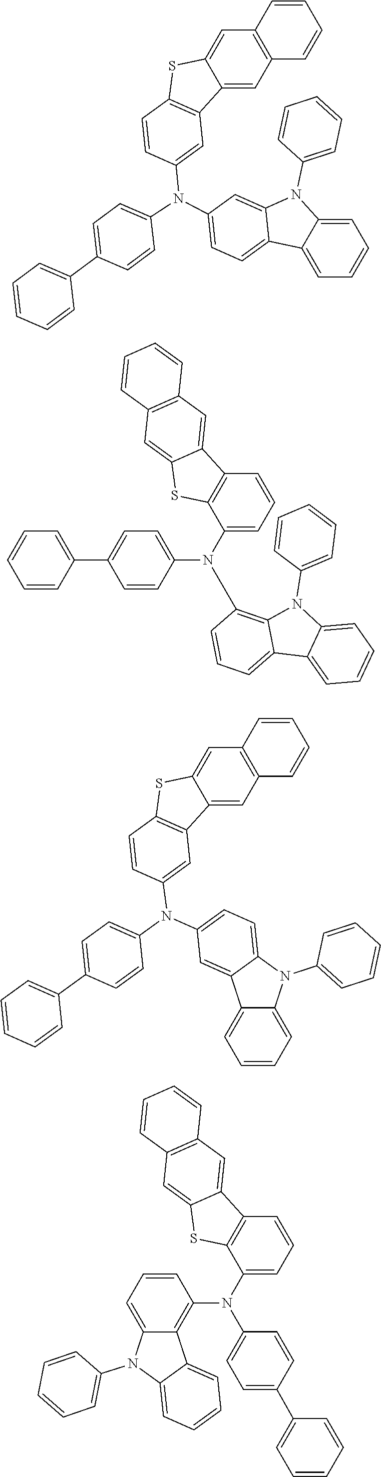

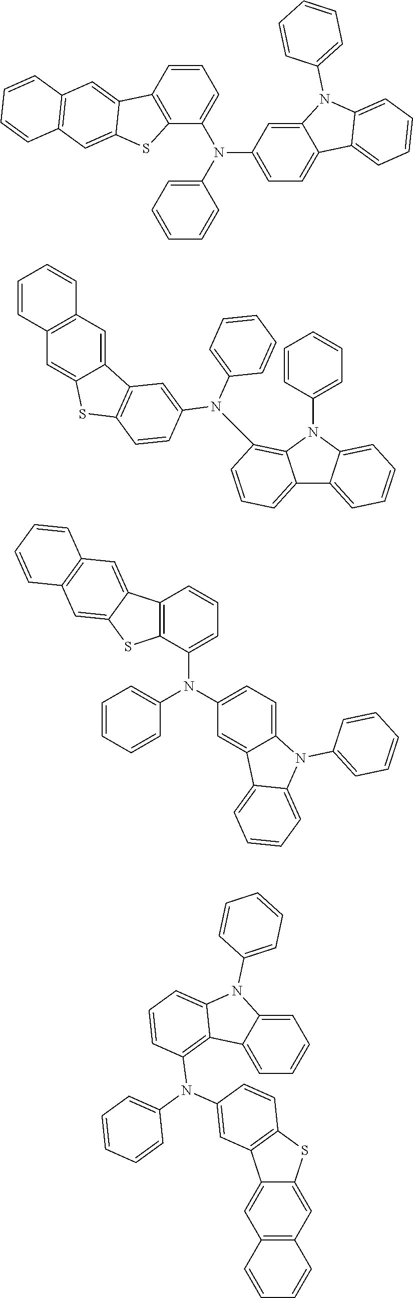 [SMILES: c1ccc(N(c2ccc3c4ccccc4n(-c4ccccc4)c3c2)c2cccc3c2sc2cc4ccccc4cc23)cc1 ; c1ccc(N(c2ccc3sc4cc5ccccc5cc4c3c2)c2cccc3c4ccccc4n(-c4ccccc4)c23)cc1 ; c1ccc(N(c2ccc3c(c2)c2ccccc2n3-c2ccccc2)c2cccc3c2sc2cc4ccccc4cc23)cc1 ; c1ccc(N(c2ccc3sc4cc5ccccc5cc4c3c2)c2cccc3c2c2ccccc2n3-c2ccccc2)cc1]

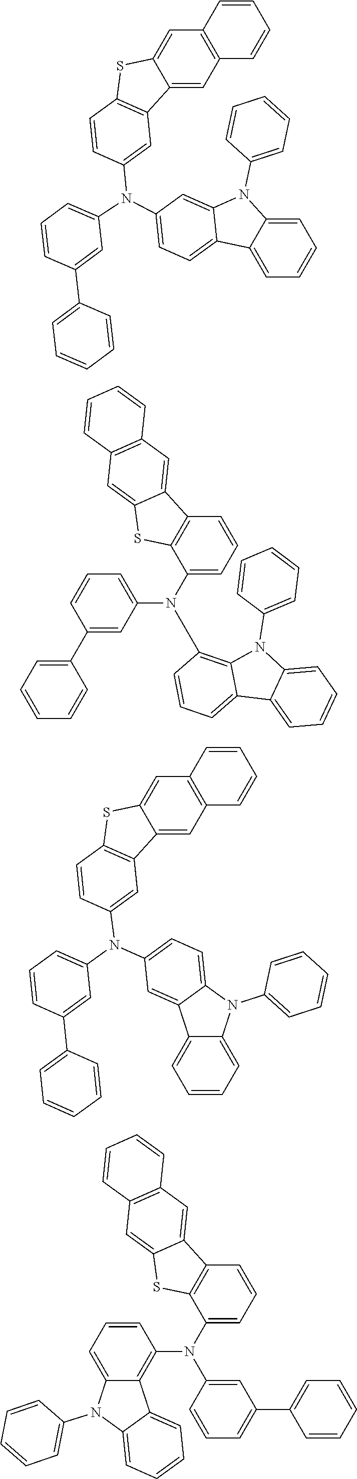

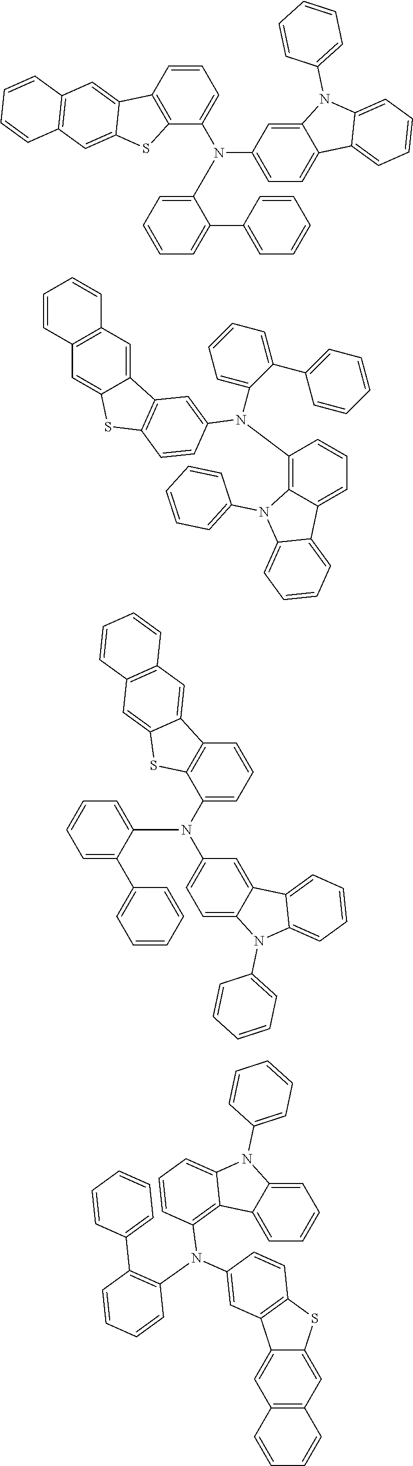

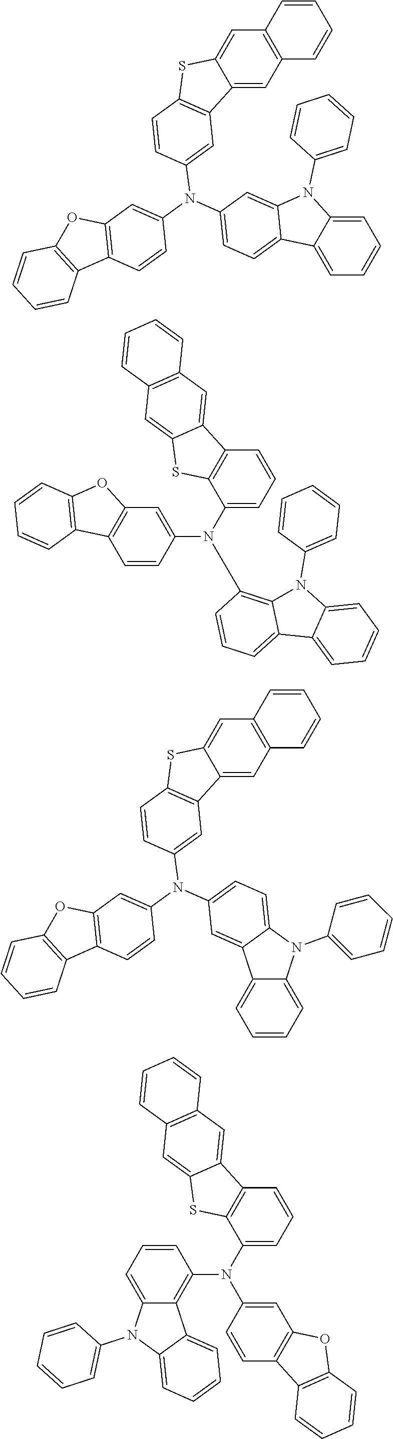

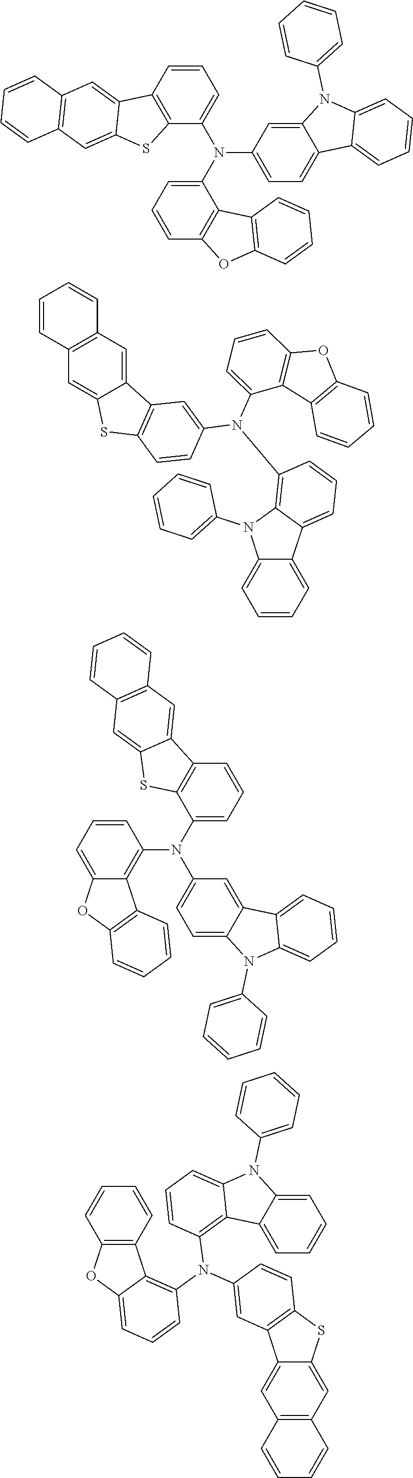

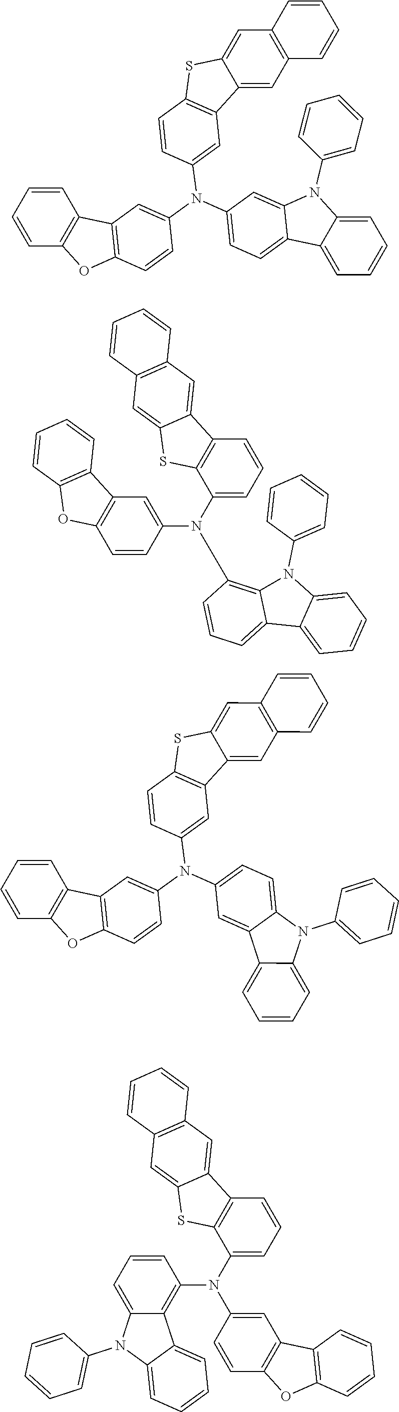

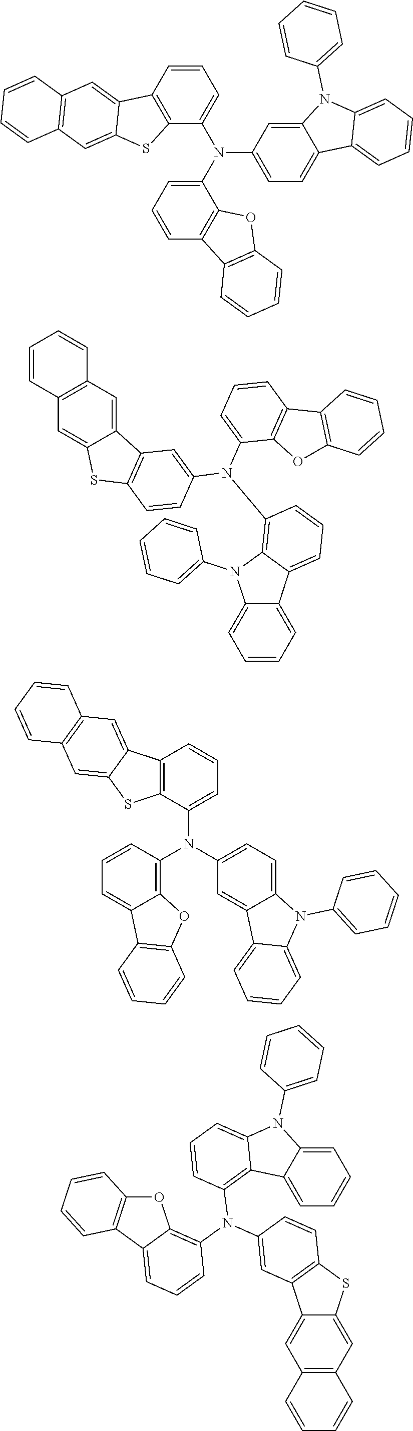

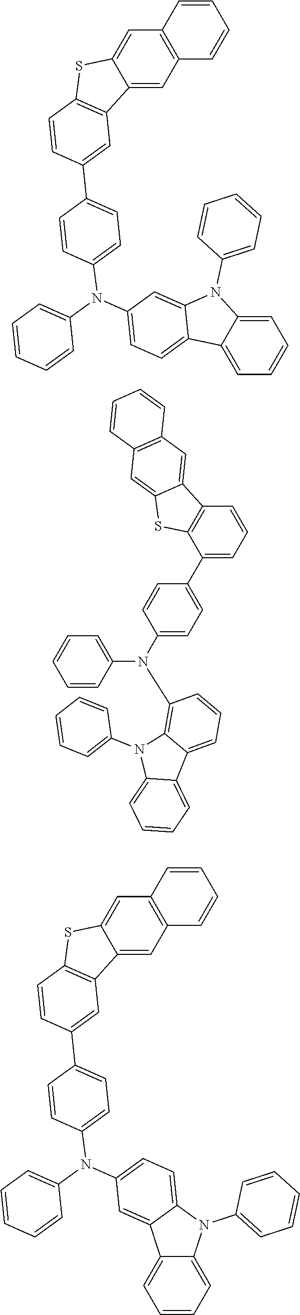

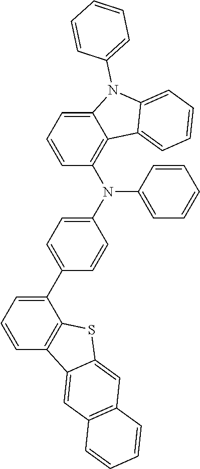 [SMILES: c1ccc(N(c2ccc(-c3cccc4c3sc3cc5ccccc5cc34)cc2)c2cccc3c2c2ccccc2n3-c2ccccc2)cc1]

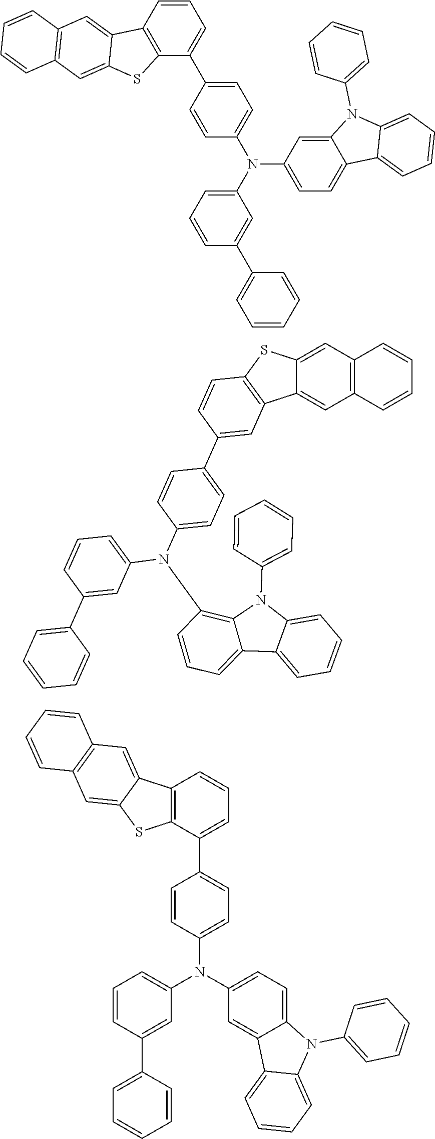

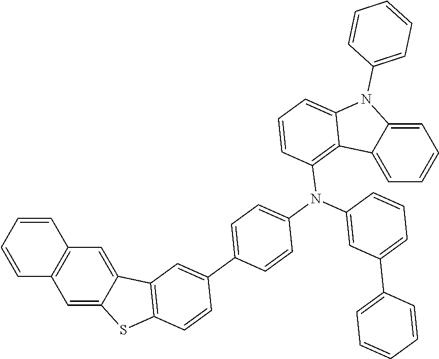 [SMILES: c1ccc(-c2cccc(N(c3ccc(-c4ccc5sc6cc7ccccc7cc6c5c4)cc3)c3cccc4c3c3ccccc3n4-c3ccccc3)c2)cc1]

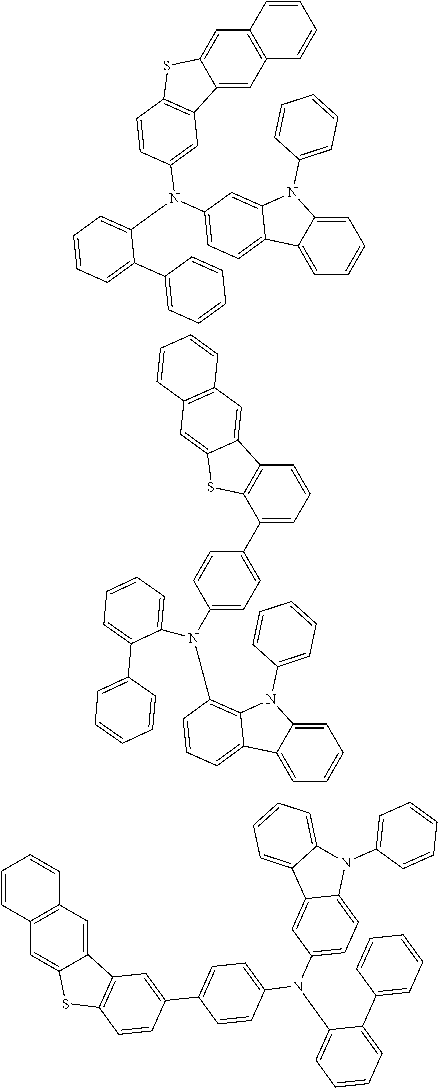

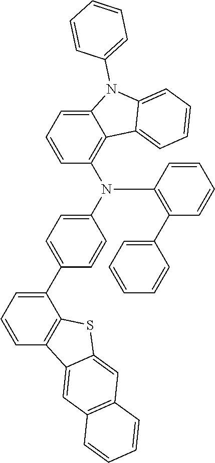 [SMILES: c1ccc(-c2ccccc2N(c2ccc(-c3cccc4c3sc3cc5ccccc5cc34)cc2)c2cccc3c2c2ccccc2n3-c2ccccc2)cc1]

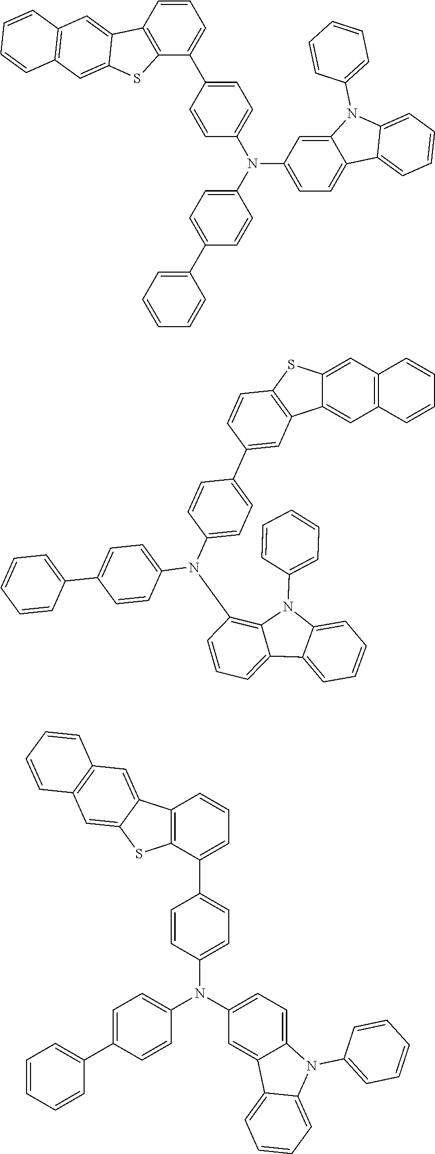 [SMILES: c1ccc(-c2ccc(N(c3ccc(-c4cccc5c4sc4cc6ccccc6cc45)cc3)c3ccc4c5ccccc5n(-c5ccccc5)c4c3)cc2)cc1 ; c1ccc(-c2ccc(N(c3ccc(-c4ccc5sc6cc7ccccc7cc6c5c4)cc3)c3cccc4c5ccccc5n(-c5ccccc5)c34)cc2)cc1 ; c1ccc(-c2ccc(N(c3ccc(-c4cccc5c4sc4cc6ccccc6cc45)cc3)c3ccc4c(c3)c3ccccc3n4-c3ccccc3)cc2)cc1]

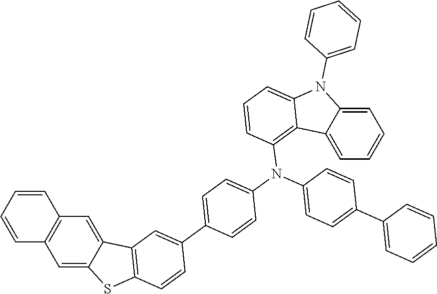 [SMILES: c1ccc(-c2ccc(N(c3ccc(-c4ccc5sc6cc7ccccc7cc6c5c4)cc3)c3cccc4c3c3ccccc3n4-c3ccccc3)cc2)cc1]

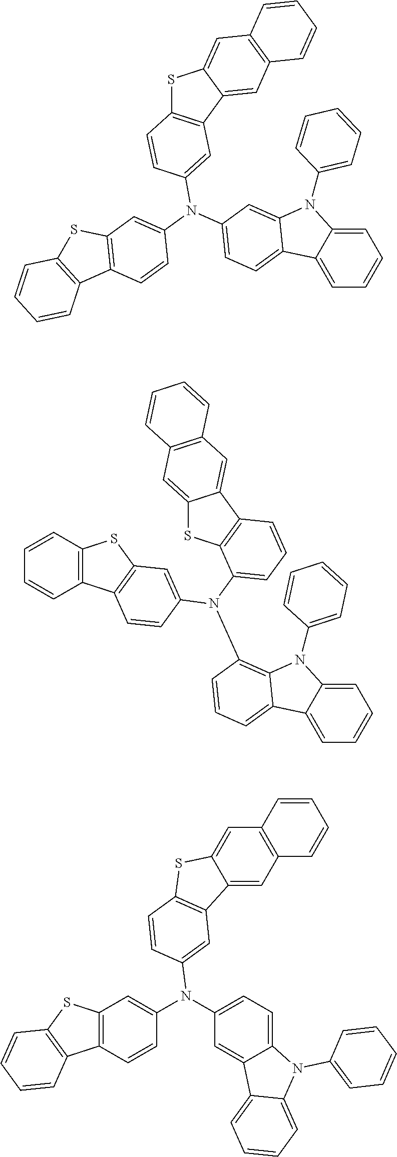 [SMILES: c1ccc(-n2c3ccccc3c3ccc(N(c4ccc5c(c4)sc4ccccc45)c4ccc5sc6cc7ccccc7cc6c5c4)cc32)cc1 ; c1ccc(-n2c3ccccc3c3cccc(N(c4ccc5c(c4)sc4ccccc45)c4cccc5c4sc4cc6ccccc6cc45)c32)cc1 ; c1ccc(-n2c3ccccc3c3cc(N(c4ccc5c(c4)sc4ccccc45)c4ccc5sc6cc7ccccc7cc6c5c4)ccc32)cc1]

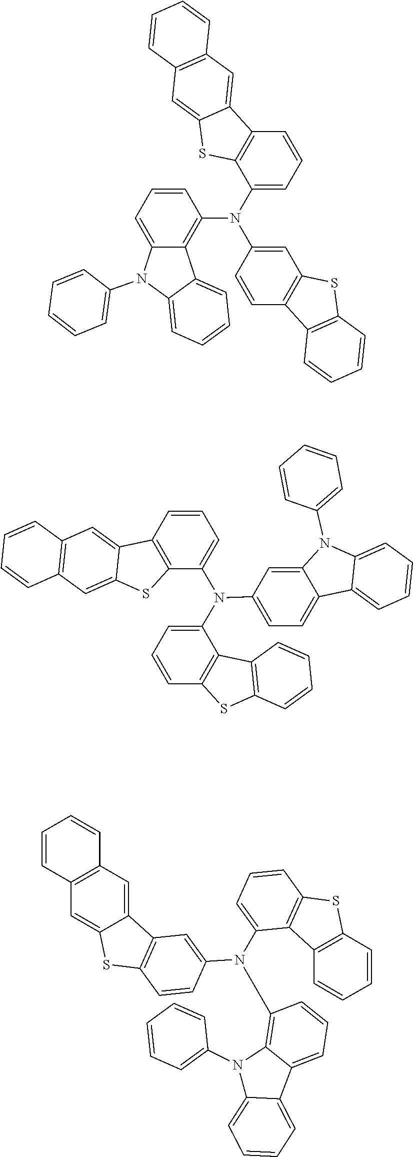 [SMILES: c1ccc(-n2c3ccccc3c3c(N(c4ccc5c(c4)sc4ccccc45)c4cccc5c4sc4cc6ccccc6cc45)cccc32)cc1 ; c1ccc(-n2c3ccccc3c3ccc(N(c4cccc5c4sc4cc6ccccc6cc45)c4cccc5sc6ccccc6c45)cc32)cc1 ; c1ccc(-n2c3ccccc3c3cccc(N(c4ccc5sc6cc7ccccc7cc6c5c4)c4cccc5sc6ccccc6c45)c32)cc1]

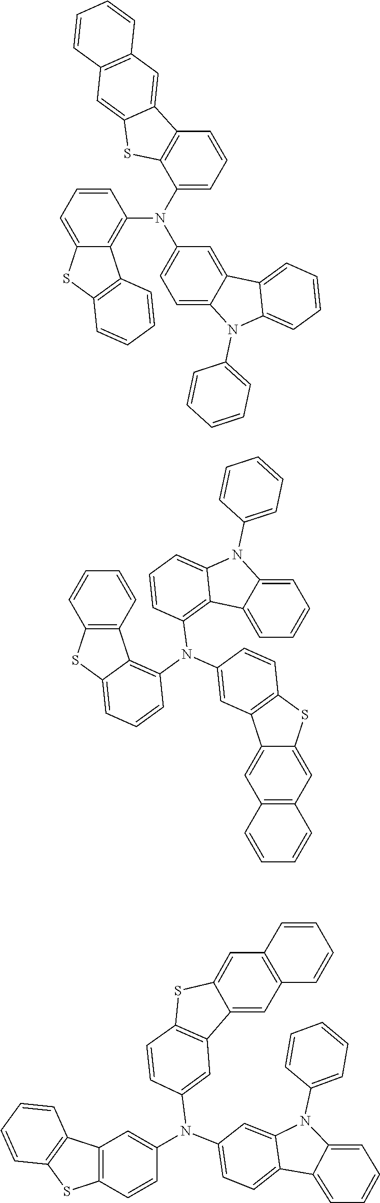

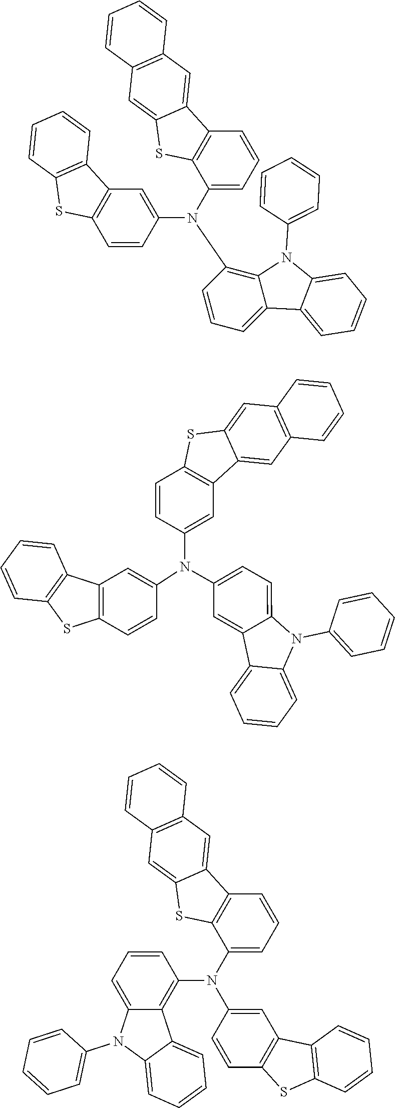 [SMILES: c1ccc(-n2c3ccccc3c3cccc(N(c4ccc5sc6ccccc6c5c4)c4cccc5c4sc4cc6ccccc6cc45)c32)cc1 ; c1ccc(-n2c3ccccc3c3cc(N(c4ccc5sc6ccccc6c5c4)c4ccc5sc6cc7ccccc7cc6c5c4)ccc32)cc1 ; c1ccc(-n2c3ccccc3c3c(N(c4ccc5sc6ccccc6c5c4)c4cccc5c4sc4cc6ccccc6cc45)cccc32)cc1]

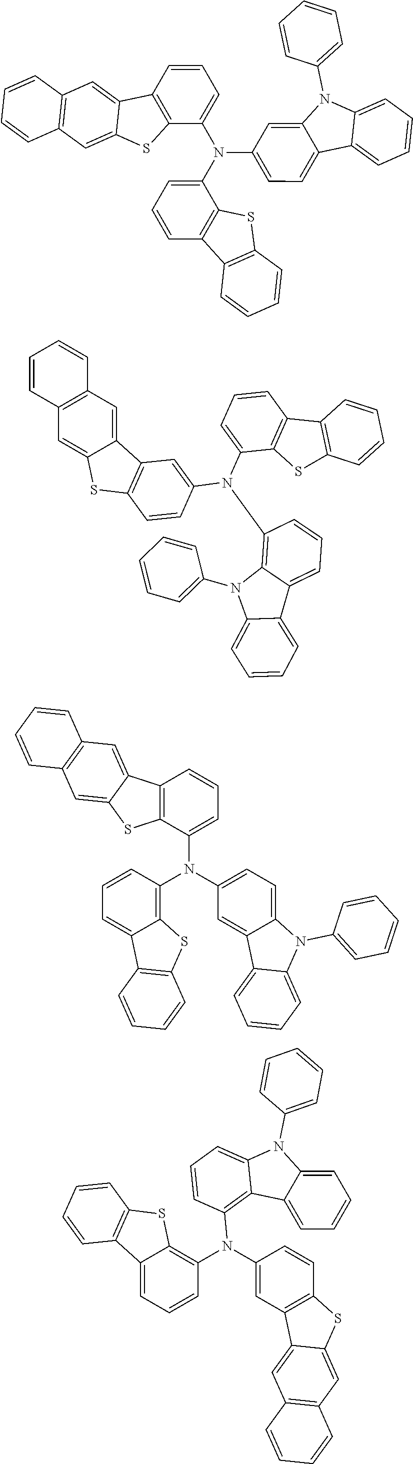

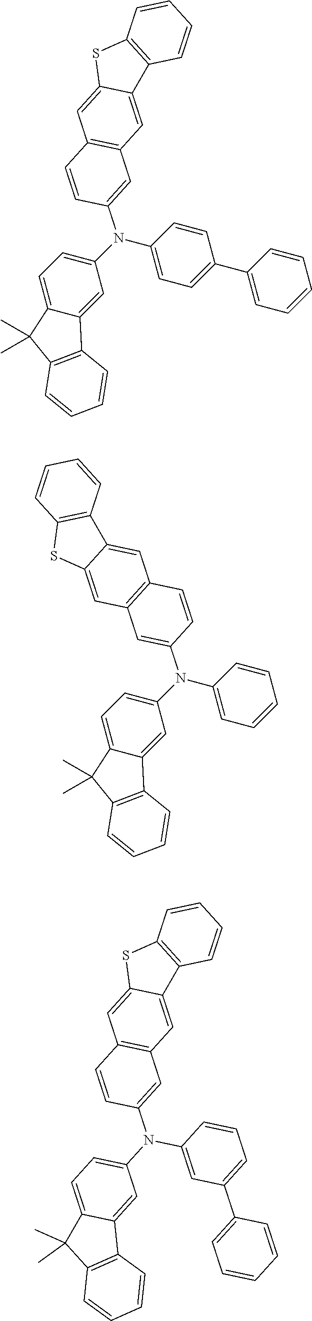

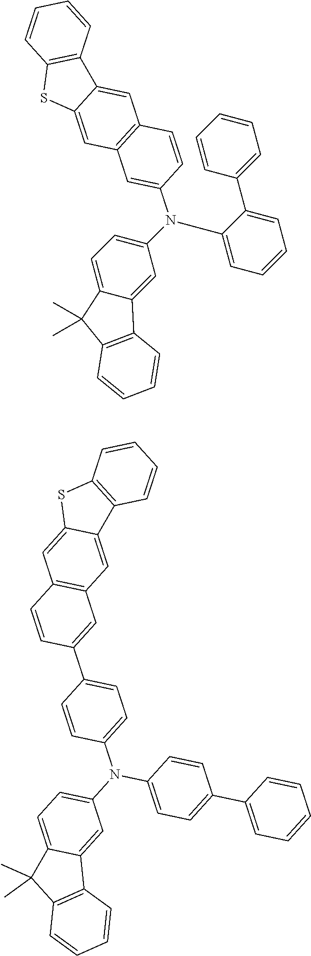

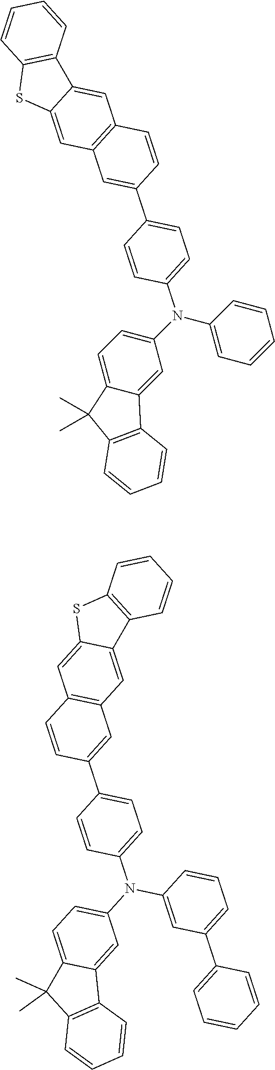 [SMILES: CC1(C)c2ccccc2-c2cc(N(c3ccccc3)c3ccc(-c4ccc5cc6c(cc5c4)sc4ccccc46)cc3)ccc21 ; CC1(C)c2ccccc2-c2cc(N(c3ccc(-c4ccc5cc6sc7ccccc7c6cc5c4)cc3)c3cccc(-c4ccccc4)c3)ccc21]

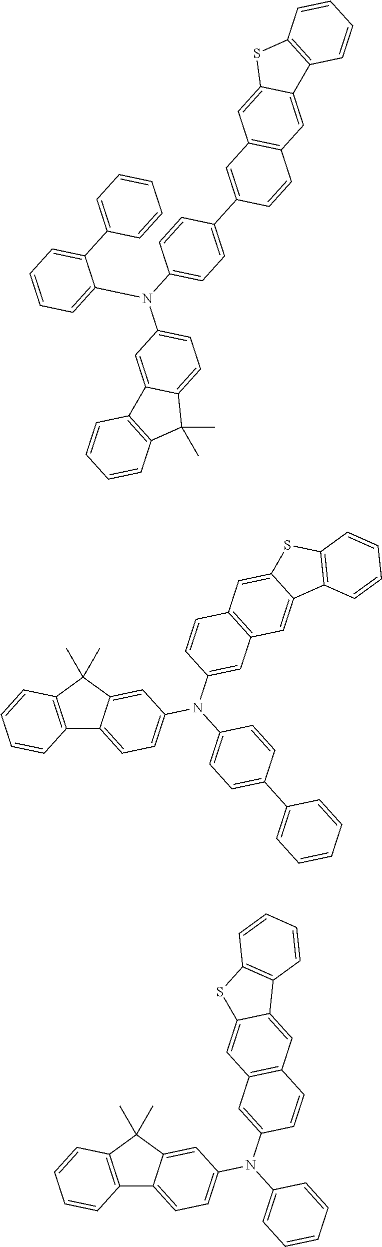 [SMILES: CC1(C)c2ccccc2-c2cc(N(c3ccc(-c4ccc5cc6c(cc5c4)sc4ccccc46)cc3)c3ccccc3-c3ccccc3)ccc21 ; CC1(C)c2ccccc2-c2ccc(N(c3ccc(-c4ccccc4)cc3)c3ccc4cc5sc6ccccc6c5cc4c3)cc21 ; CC1(C)c2ccccc2-c2ccc(N(c3ccccc3)c3ccc4cc5c(cc4c3)sc3ccccc35)cc21]

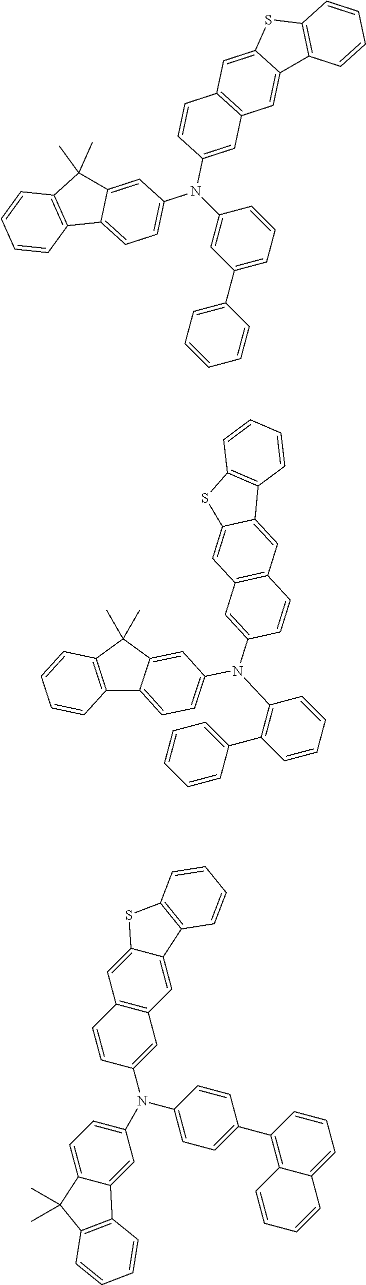 [SMILES: CC1(C)c2ccccc2-c2ccc(N(c3cccc(-c4ccccc4)c3)c3ccc4cc5sc6ccccc6c5cc4c3)cc21 ; CC1(C)c2ccccc2-c2ccc(N(c3ccc4cc5c(cc4c3)sc3ccccc35)c3ccccc3-c3ccccc3)cc21 ; CC1(C)c2ccccc2-c2cc(N(c3ccc(-c4cccc5ccccc45)cc3)c3ccc4cc5sc6ccccc6c5cc4c3)ccc21]

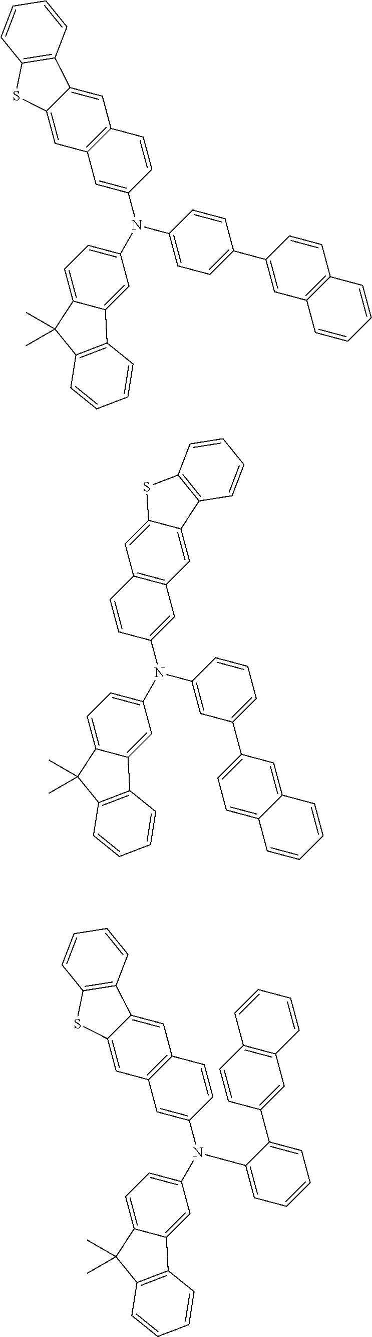 [SMILES: CC1(C)c2ccccc2-c2cc(N(c3ccc(-c4ccc5ccccc5c4)cc3)c3ccc4cc5c(cc4c3)sc3ccccc35)ccc21 ; CC1(C)c2ccccc2-c2cc(N(c3cccc(-c4ccc5ccccc5c4)c3)c3ccc4cc5sc6ccccc6c5cc4c3)ccc21 ; CC1(C)c2ccccc2-c2cc(N(c3ccc4cc5c(cc4c3)sc3ccccc35)c3ccccc3-c3ccc4ccccc4c3)ccc21]

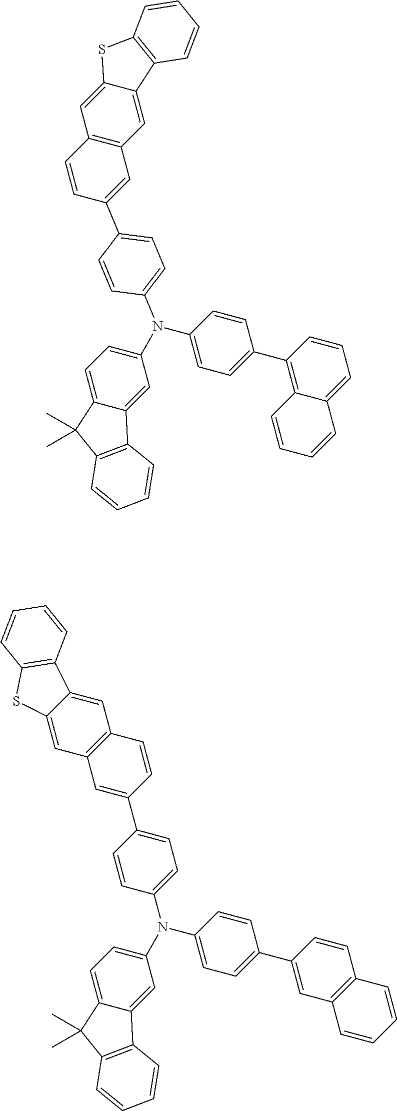 [SMILES: CC1(C)c2ccccc2-c2cc(N(c3ccc(-c4ccc5cc6sc7ccccc7c6cc5c4)cc3)c3ccc(-c4cccc5ccccc45)cc3)ccc21 ; CC1(C)c2ccccc2-c2cc(N(c3ccc(-c4ccc5ccccc5c4)cc3)c3ccc(-c4ccc5cc6c(cc5c4)sc4ccccc46)cc3)ccc21]

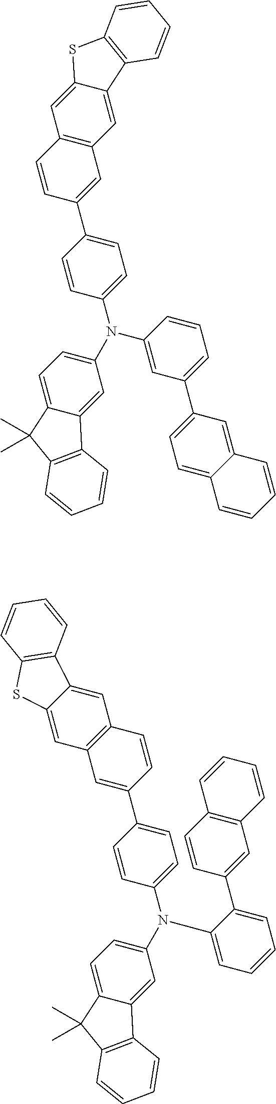 [SMILES: CC1(C)c2ccccc2-c2cc(N(c3ccc(-c4ccc5cc6sc7ccccc7c6cc5c4)cc3)c3cccc(-c4ccc5ccccc5c4)c3)ccc21 ; CC1(C)c2ccccc2-c2cc(N(c3ccc(-c4ccc5cc6c(cc5c4)sc4ccccc46)cc3)c3ccccc3-c3ccc4ccccc4c3)ccc21]

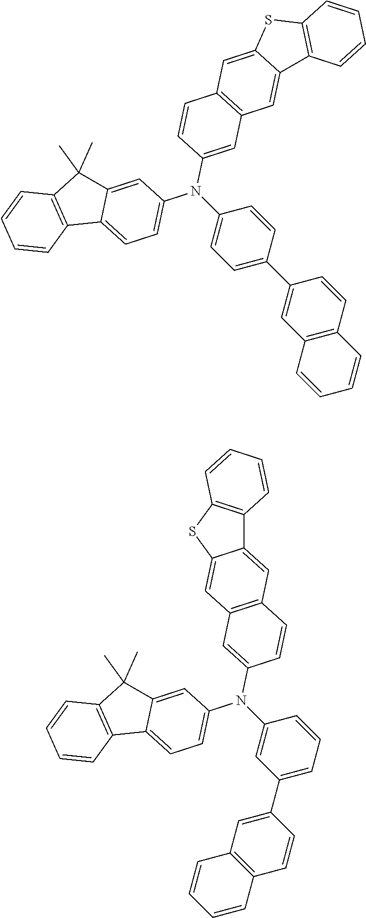 [SMILES: CC1(C)c2ccccc2-c2ccc(N(c3ccc(-c4ccc5ccccc5c4)cc3)c3ccc4cc5sc6ccccc6c5cc4c3)cc21 ; CC1(C)c2ccccc2-c2ccc(N(c3cccc(-c4ccc5ccccc5c4)c3)c3ccc4cc5c(cc4c3)sc3ccccc35)cc21]

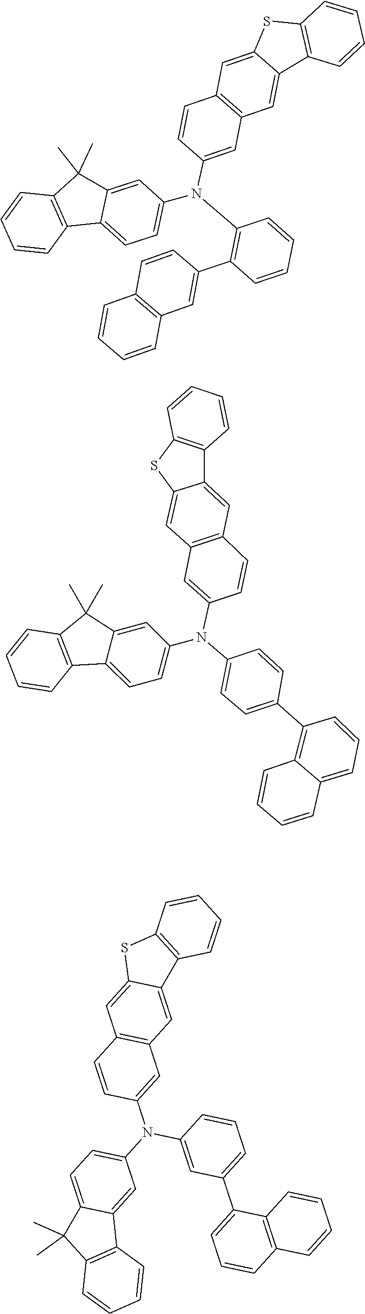 [SMILES: CC1(C)c2ccccc2-c2ccc(N(c3ccc4cc5sc6ccccc6c5cc4c3)c3ccccc3-c3ccc4ccccc4c3)cc21 ; CC1(C)c2ccccc2-c2ccc(N(c3ccc(-c4cccc5ccccc45)cc3)c3ccc4cc5c(cc4c3)sc3ccccc35)cc21 ; CC1(C)c2ccccc2-c2cc(N(c3cccc(-c4cccc5ccccc45)c3)c3ccc4cc5sc6ccccc6c5cc4c3)ccc21]

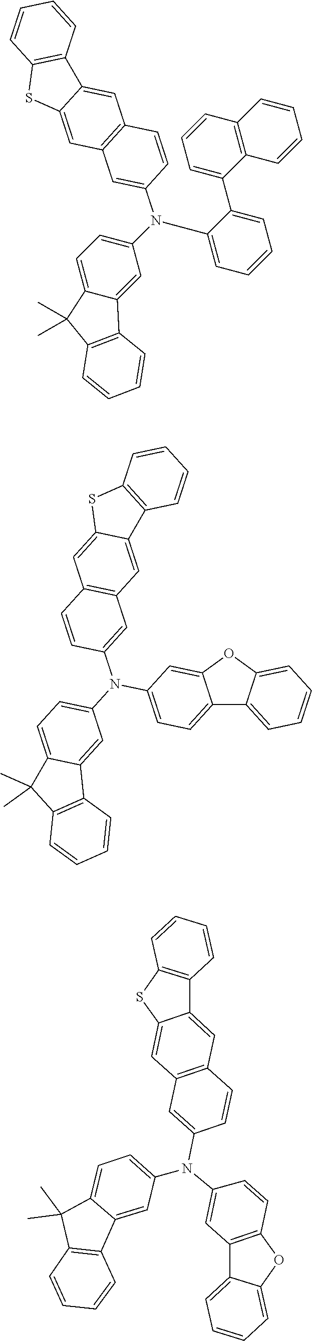 [SMILES: CC1(C)c2ccccc2-c2cc(N(c3ccc4cc5c(cc4c3)sc3ccccc35)c3ccccc3-c3cccc4ccccc34)ccc21 ; CC1(C)c2ccccc2-c2cc(N(c3ccc4cc5sc6ccccc6c5cc4c3)c3ccc4c(c3)oc3ccccc34)ccc21 ; CC1(C)c2ccccc2-c2cc(N(c3ccc4cc5c(cc4c3)sc3ccccc35)c3ccc4oc5ccccc5c4c3)ccc21]

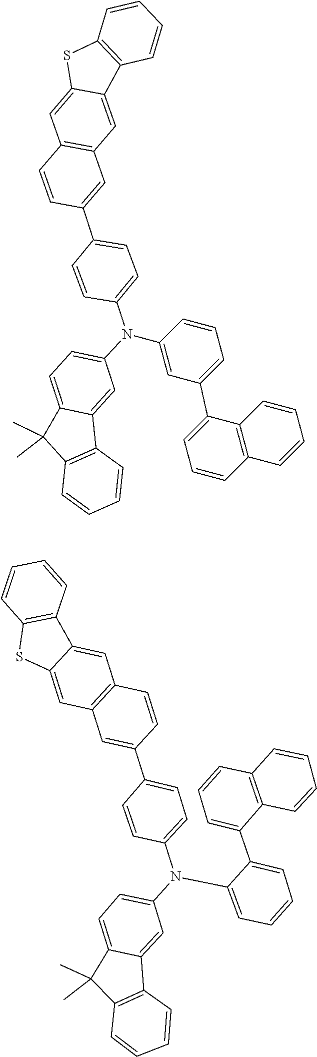 [SMILES: CC1(C)c2ccccc2-c2cc(N(c3ccc(-c4ccc5cc6sc7ccccc7c6cc5c4)cc3)c3cccc(-c4cccc5ccccc45)c3)ccc21 ; CC1(C)c2ccccc2-c2cc(N(c3ccc(-c4ccc5cc6c(cc5c4)sc4ccccc46)cc3)c3ccccc3-c3cccc4ccccc34)ccc21]

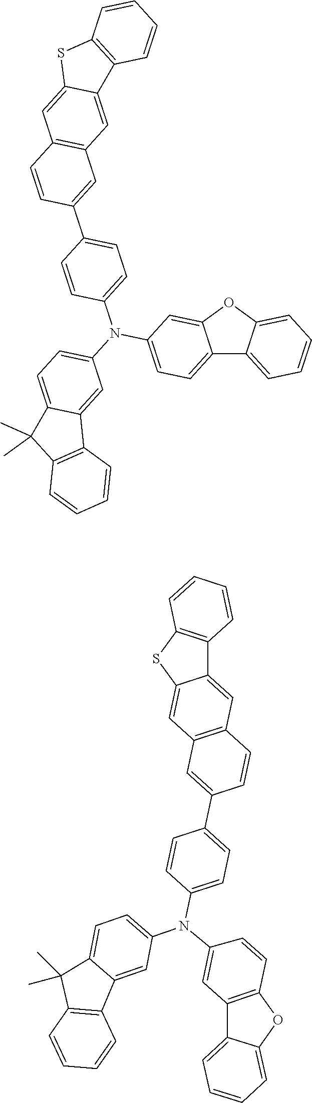 [SMILES: CC1(C)c2ccccc2-c2cc(N(c3ccc(-c4ccc5cc6sc7ccccc7c6cc5c4)cc3)c3ccc4c(c3)oc3ccccc34)ccc21 ; CC1(C)c2ccccc2-c2cc(N(c3ccc(-c4ccc5cc6c(cc5c4)sc4ccccc46)cc3)c3ccc4oc5ccccc5c4c3)ccc21]

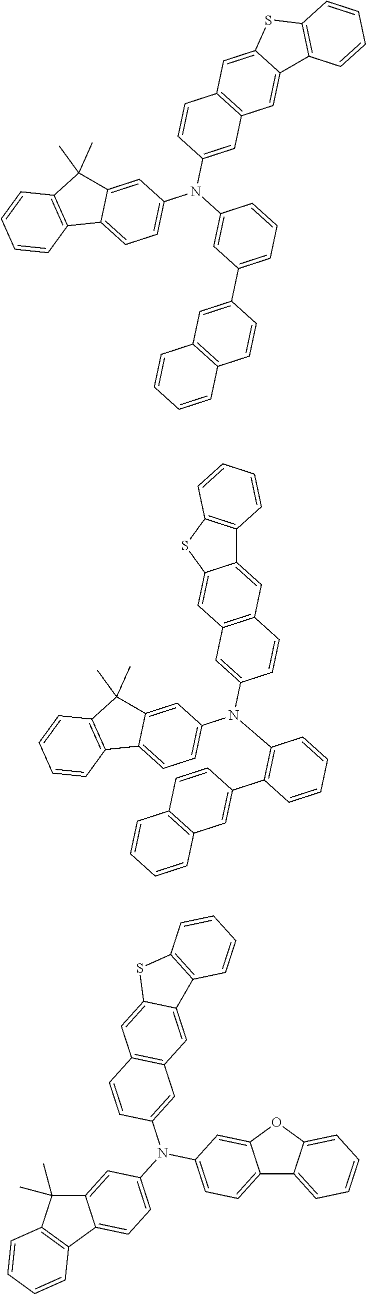 [SMILES: CC1(C)c2ccccc2-c2ccc(N(c3cccc(-c4ccc5ccccc5c4)c3)c3ccc4cc5sc6ccccc6c5cc4c3)cc21 ; CC1(C)c2ccccc2-c2ccc(N(c3ccc4cc5c(cc4c3)sc3ccccc35)c3ccccc3-c3ccc4ccccc4c3)cc21 ; CC1(C)c2ccccc2-c2ccc(N(c3ccc4cc5sc6ccccc6c5cc4c3)c3ccc4c(c3)oc3ccccc34)cc21]

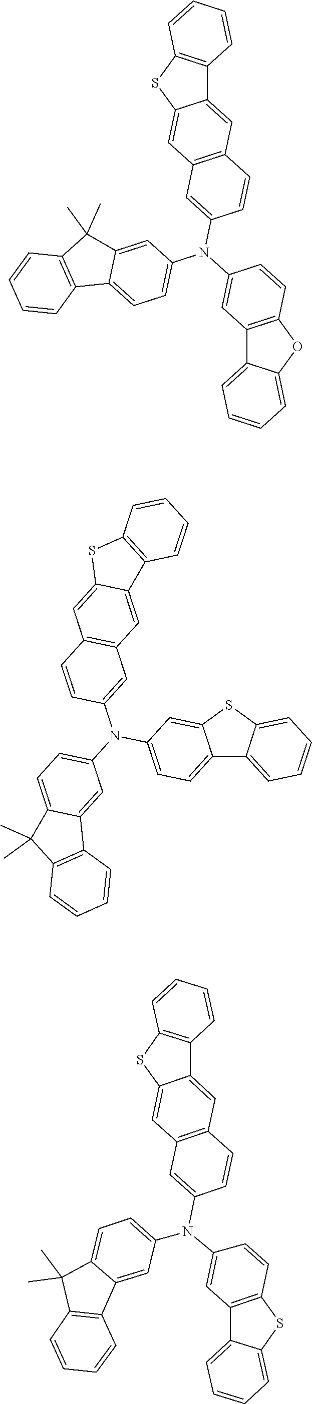 [SMILES: CC1(C)c2ccccc2-c2ccc(N(c3ccc4cc5c(cc4c3)sc3ccccc35)c3ccc4oc5ccccc5c4c3)cc21 ; CC1(C)c2ccccc2-c2cc(N(c3ccc4cc5sc6ccccc6c5cc4c3)c3ccc4c(c3)sc3ccccc34)ccc21 ; CC1(C)c2ccccc2-c2cc(N(c3ccc4cc5c(cc4c3)sc3ccccc35)c3ccc4sc5ccccc5c4c3)ccc21]

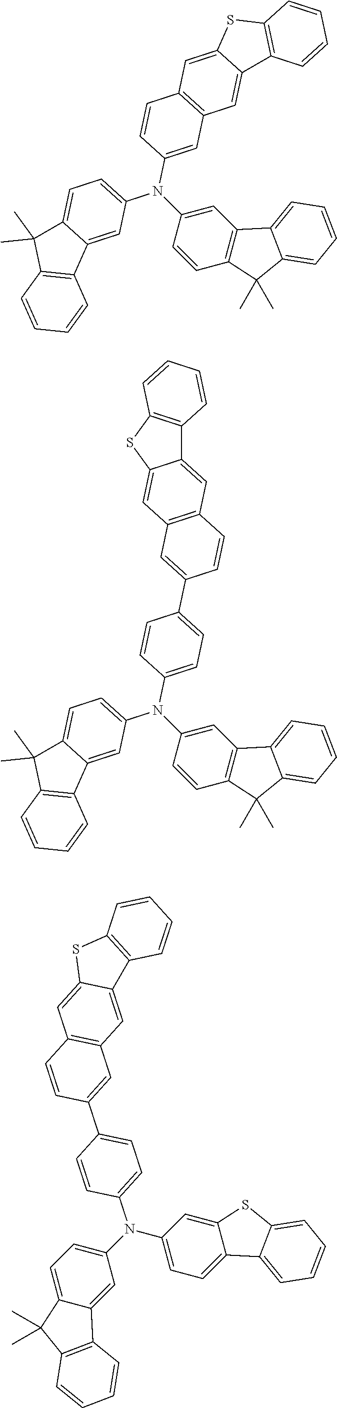 [SMILES: CC1(C)c2ccccc2-c2cc(N(c3ccc4c(c3)-c3ccccc3C4(C)C)c3ccc4cc5sc6ccccc6c5cc4c3)ccc21 ; CC1(C)c2ccccc2-c2cc(N(c3ccc(-c4ccc5cc6c(cc5c4)sc4ccccc46)cc3)c3ccc4c(c3)-c3ccccc3C4(C)C)ccc21 ; CC1(C)c2ccccc2-c2cc(N(c3ccc(-c4ccc5cc6sc7ccccc7c6cc5c4)cc3)c3ccc4c(c3)sc3ccccc34)ccc21]

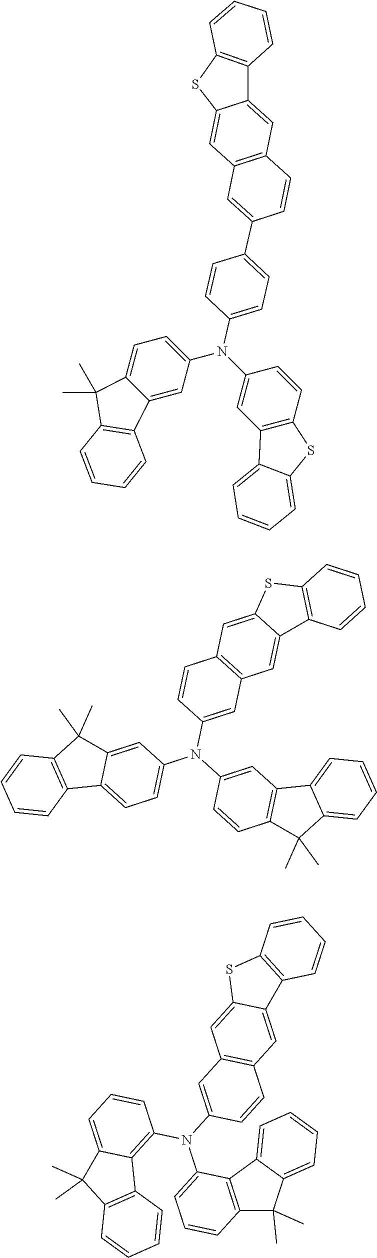 [SMILES: CC1(C)c2ccccc2-c2cc(N(c3ccc(-c4ccc5cc6c(cc5c4)sc4ccccc46)cc3)c3ccc4sc5ccccc5c4c3)ccc21 ; CC1(C)c2ccccc2-c2cc(N(c3ccc4c(c3)C(C)(C)c3ccccc3-4)c3ccc4cc5sc6ccccc6c5cc4c3)ccc21 ; CC1(C)c2ccccc2-c2c(N(c3ccc4cc5c(cc4c3)sc3ccccc35)c3cccc4c3-c3ccccc3C4(C)C)cccc21]

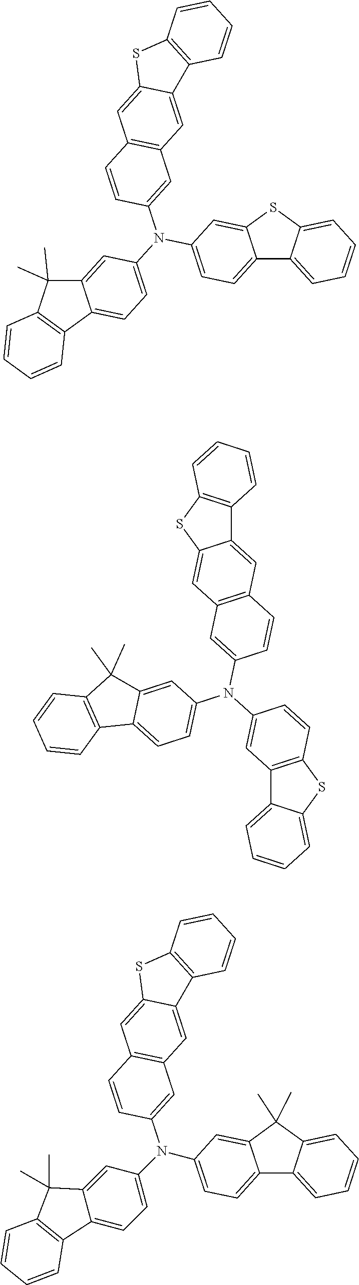 [SMILES: CC1(C)c2ccccc2-c2ccc(N(c3ccc4cc5sc6ccccc6c5cc4c3)c3ccc4c(c3)sc3ccccc34)cc21 ; CC1(C)c2ccccc2-c2ccc(N(c3ccc4cc5c(cc4c3)sc3ccccc35)c3ccc4sc5ccccc5c4c3)cc21 ; CC1(C)c2ccccc2-c2ccc(N(c3ccc4c(c3)C(C)(C)c3ccccc3-4)c3ccc4cc5sc6ccccc6c5cc4c3)cc21]

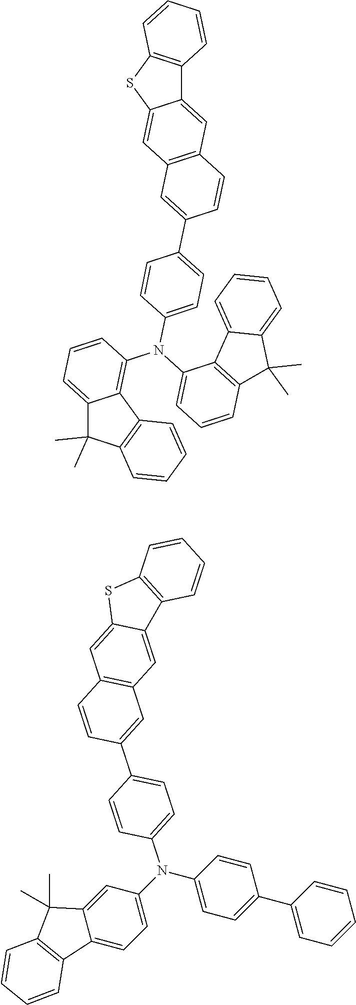

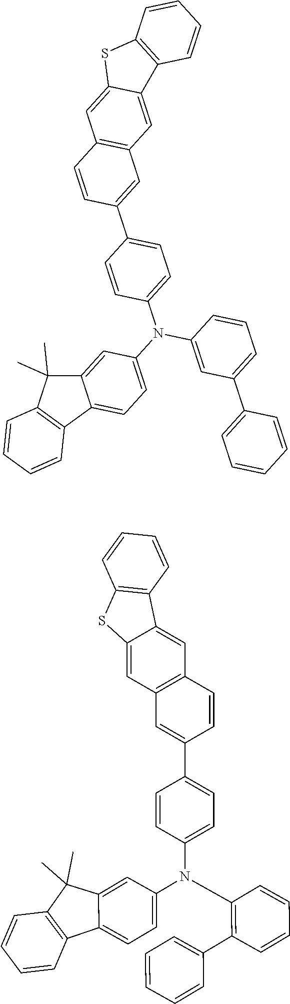 [SMILES: CC1(C)c2ccccc2-c2ccc(N(c3ccc(-c4ccc5cc6sc7ccccc7c6cc5c4)cc3)c3cccc(-c4ccccc4)c3)cc21 ; CC1(C)c2ccccc2-c2ccc(N(c3ccc(-c4ccc5cc6c(cc5c4)sc4ccccc46)cc3)c3ccccc3-c3ccccc3)cc21]

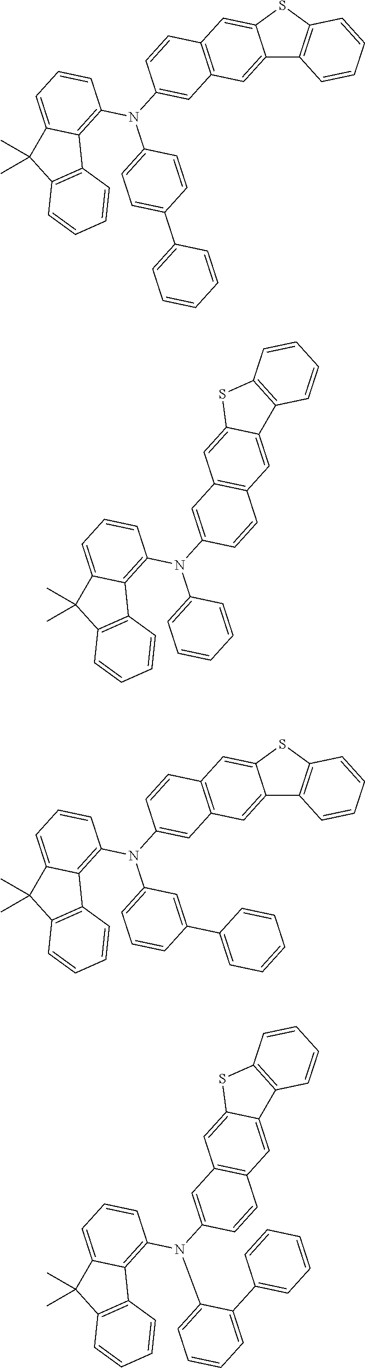 [SMILES: CC1(C)c2ccccc2-c2c(N(c3ccc(-c4ccccc4)cc3)c3ccc4cc5sc6ccccc6c5cc4c3)cccc21 ; CC1(C)c2ccccc2-c2c(N(c3ccccc3)c3ccc4cc5c(cc4c3)sc3ccccc35)cccc21 ; CC1(C)c2ccccc2-c2c(N(c3cccc(-c4ccccc4)c3)c3ccc4cc5sc6ccccc6c5cc4c3)cccc21 ; CC1(C)c2ccccc2-c2c(N(c3ccc4cc5c(cc4c3)sc3ccccc35)c3ccccc3-c3ccccc3)cccc21]

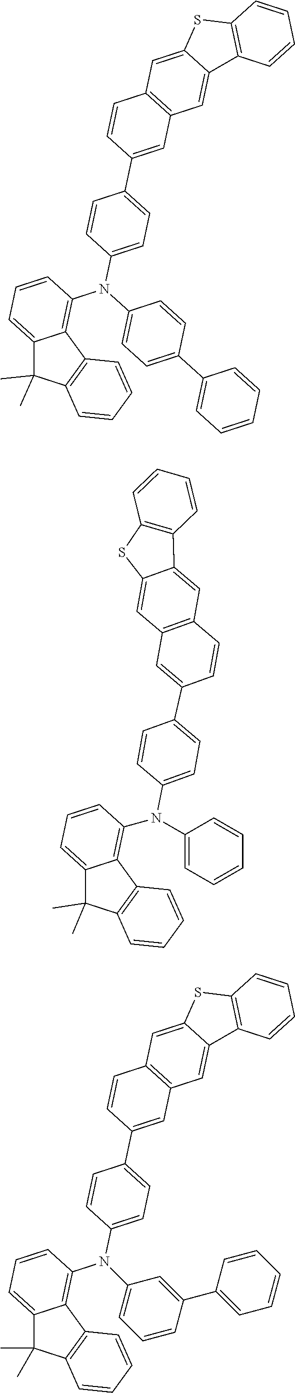 [SMILES: CC1(C)c2ccccc2-c2c(N(c3ccc(-c4ccccc4)cc3)c3ccc(-c4ccc5cc6sc7ccccc7c6cc5c4)cc3)cccc21 ; CC1(C)c2ccccc2-c2c(N(c3ccccc3)c3ccc(-c4ccc5cc6c(cc5c4)sc4ccccc46)cc3)cccc21 ; CC1(C)c2ccccc2-c2c(N(c3ccc(-c4ccc5cc6sc7ccccc7c6cc5c4)cc3)c3cccc(-c4ccccc4)c3)cccc21]

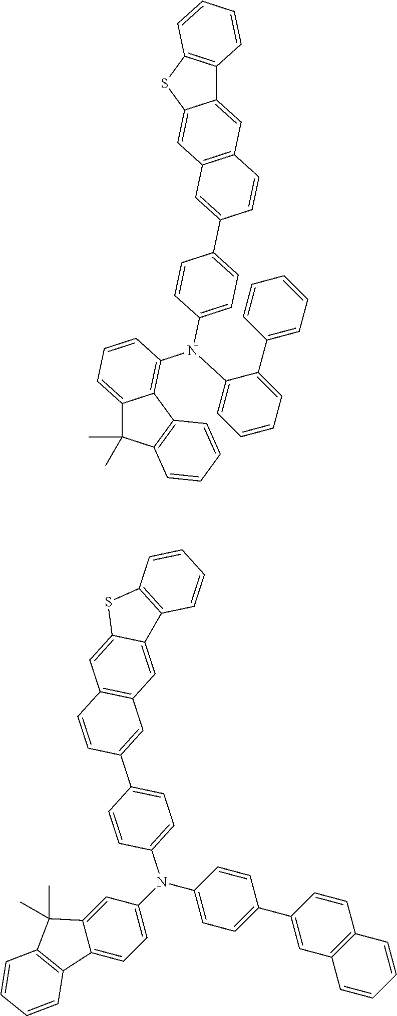 [SMILES: CC1(C)c2ccccc2-c2c(N(c3ccc(-c4ccc5cc6c(cc5c4)sc4ccccc46)cc3)c3ccccc3-c3ccccc3)cccc21 ; CC1(C)c2ccccc2-c2ccc(N(c3ccc(-c4ccc5ccccc5c4)cc3)c3ccc(-c4ccc5cc6sc7ccccc7c6cc5c4)cc3)cc21]

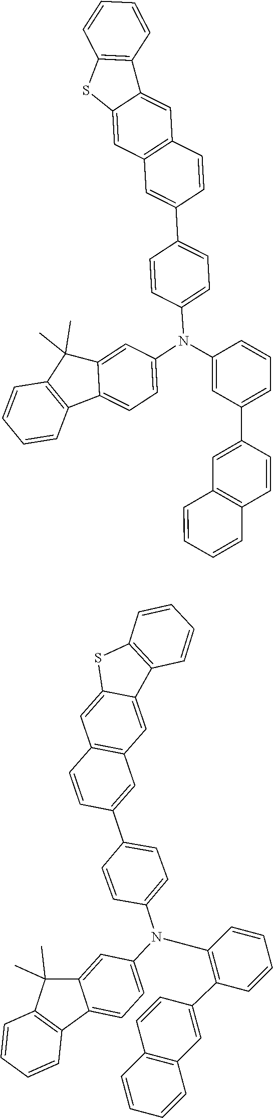 [SMILES: CC1(C)c2ccccc2-c2ccc(N(c3ccc(-c4ccc5cc6c(cc5c4)sc4ccccc46)cc3)c3cccc(-c4ccc5ccccc5c4)c3)cc21 ; CC1(C)c2ccccc2-c2ccc(N(c3ccc(-c4ccc5cc6sc7ccccc7c6cc5c4)cc3)c3ccccc3-c3ccc4ccccc4c3)cc21]

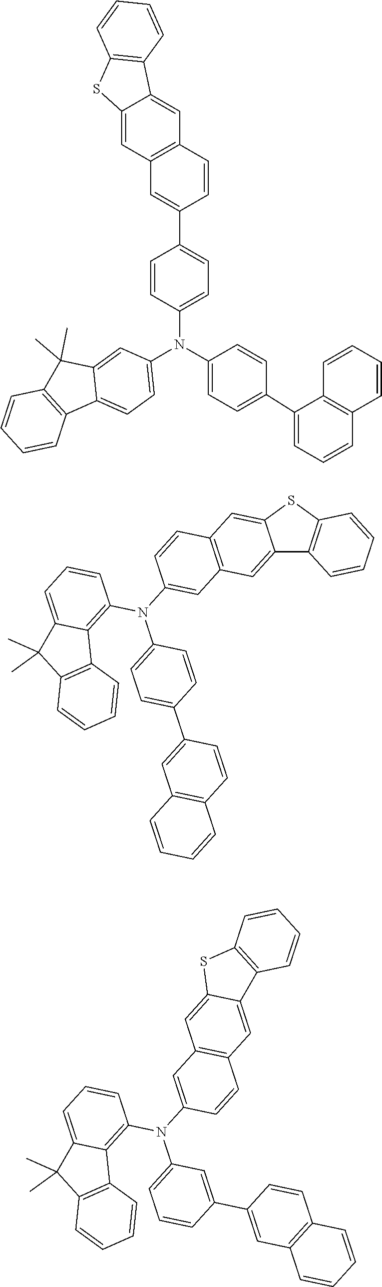 [SMILES: CC1(C)c2ccccc2-c2ccc(N(c3ccc(-c4ccc5cc6c(cc5c4)sc4ccccc46)cc3)c3ccc(-c4cccc5ccccc45)cc3)cc21 ; CC1(C)c2ccccc2-c2c(N(c3ccc(-c4ccc5ccccc5c4)cc3)c3ccc4cc5sc6ccccc6c5cc4c3)cccc21 ; CC1(C)c2ccccc2-c2c(N(c3cccc(-c4ccc5ccccc5c4)c3)c3ccc4cc5c(cc4c3)sc3ccccc35)cccc21]

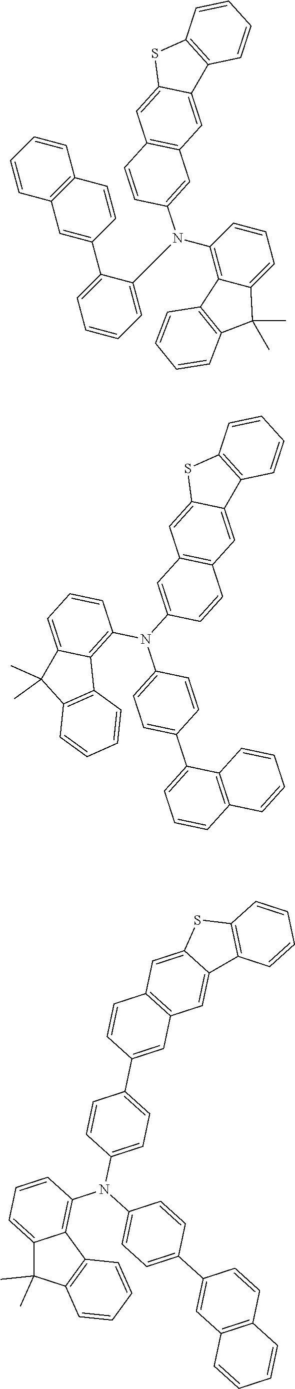

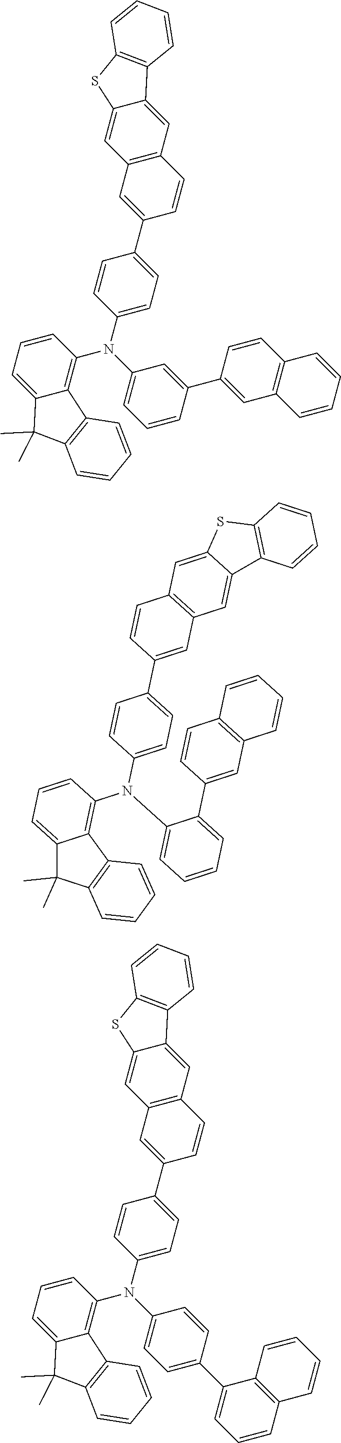

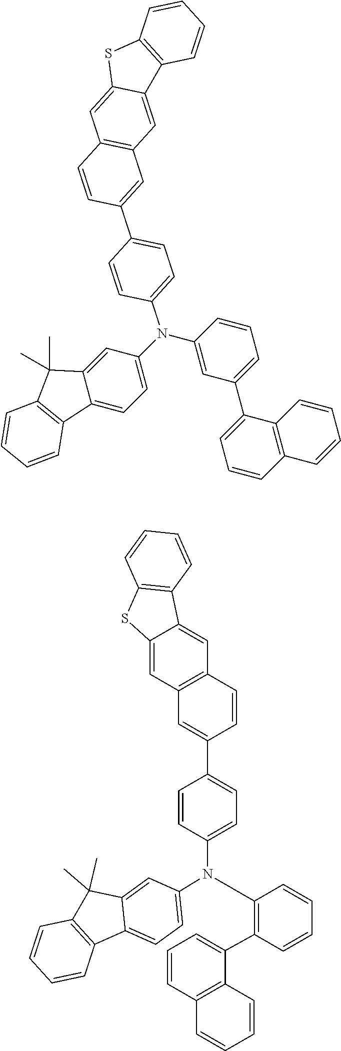 [SMILES: CC1(C)c2ccccc2-c2ccc(N(c3ccc(-c4ccc5cc6sc7ccccc7c6cc5c4)cc3)c3cccc(-c4cccc5ccccc45)c3)cc21 ; CC1(C)c2ccccc2-c2ccc(N(c3ccc(-c4ccc5cc6c(cc5c4)sc4ccccc46)cc3)c3ccccc3-c3cccc4ccccc34)cc21]

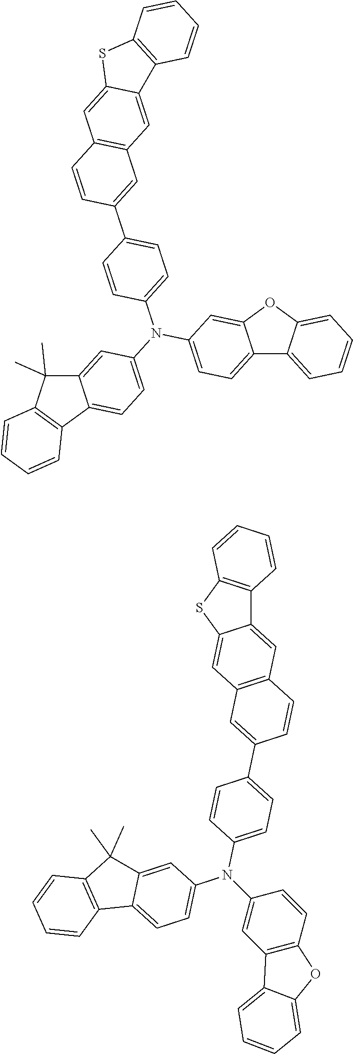 [SMILES: CC1(C)c2ccccc2-c2ccc(N(c3ccc(-c4ccc5cc6sc7ccccc7c6cc5c4)cc3)c3ccc4c(c3)oc3ccccc34)cc21 ; CC1(C)c2ccccc2-c2ccc(N(c3ccc(-c4ccc5cc6c(cc5c4)sc4ccccc46)cc3)c3ccc4oc5ccccc5c4c3)cc21]

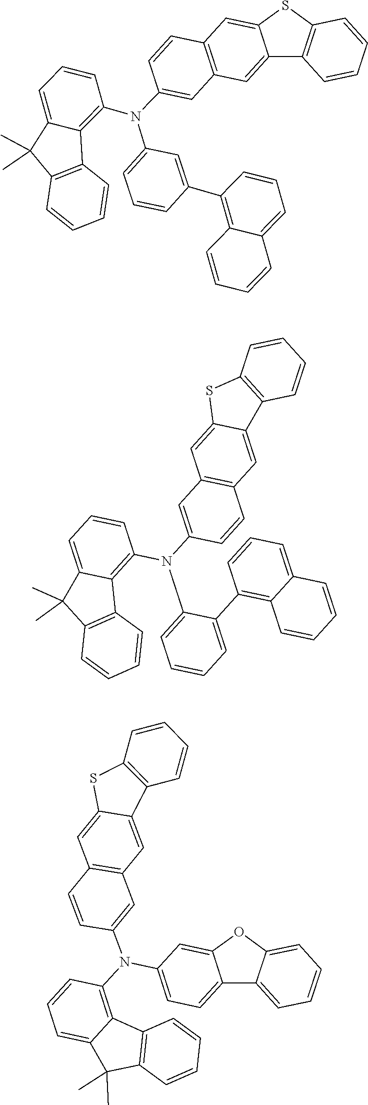 [SMILES: CC1(C)c2ccccc2-c2c(N(c3cccc(-c4cccc5ccccc45)c3)c3ccc4cc5sc6ccccc6c5cc4c3)cccc21 ; CC1(C)c2ccccc2-c2c(N(c3ccc4cc5c(cc4c3)sc3ccccc35)c3ccccc3-c3cccc4ccccc34)cccc21 ; CC1(C)c2ccccc2-c2c(N(c3ccc4cc5sc6ccccc6c5cc4c3)c3ccc4c(c3)oc3ccccc34)cccc21]

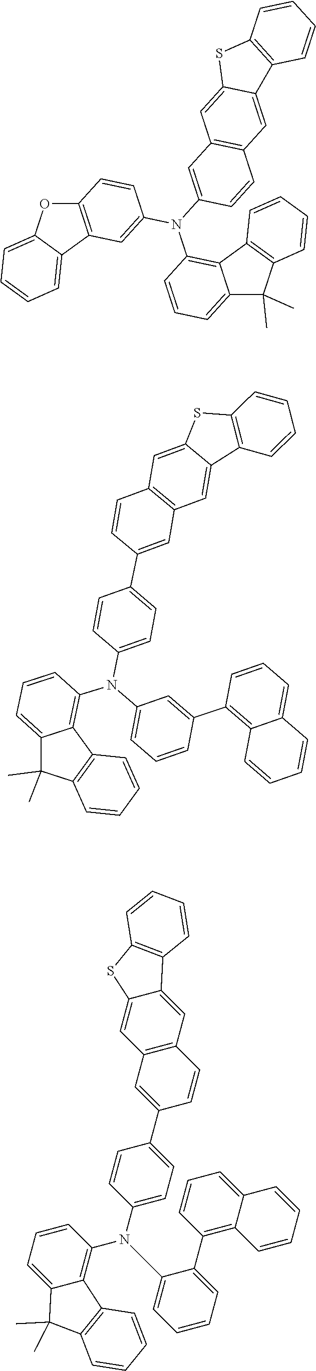 [SMILES: CC1(C)c2ccccc2-c2c(N(c3ccc4cc5c(cc4c3)sc3ccccc35)c3ccc4oc5ccccc5c4c3)cccc21 ; CC1(C)c2ccccc2-c2c(N(c3ccc(-c4ccc5cc6sc7ccccc7c6cc5c4)cc3)c3cccc(-c4cccc5ccccc45)c3)cccc21 ; CC1(C)c2ccccc2-c2c(N(c3ccc(-c4ccc5cc6c(cc5c4)sc4ccccc46)cc3)c3ccccc3-c3cccc4ccccc34)cccc21]

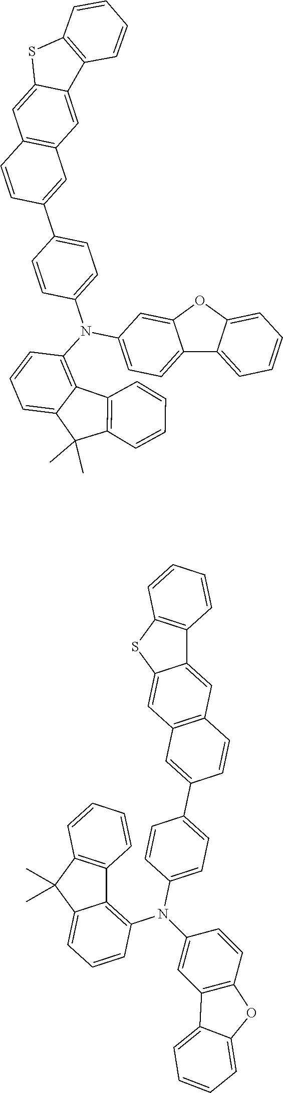 [SMILES: CC1(C)c2ccccc2-c2c(N(c3ccc(-c4ccc5cc6sc7ccccc7c6cc5c4)cc3)c3ccc4c(c3)oc3ccccc34)cccc21 ; CC1(C)c2ccccc2-c2c(N(c3ccc(-c4ccc5cc6c(cc5c4)sc4ccccc46)cc3)c3ccc4oc5ccccc5c4c3)cccc21]

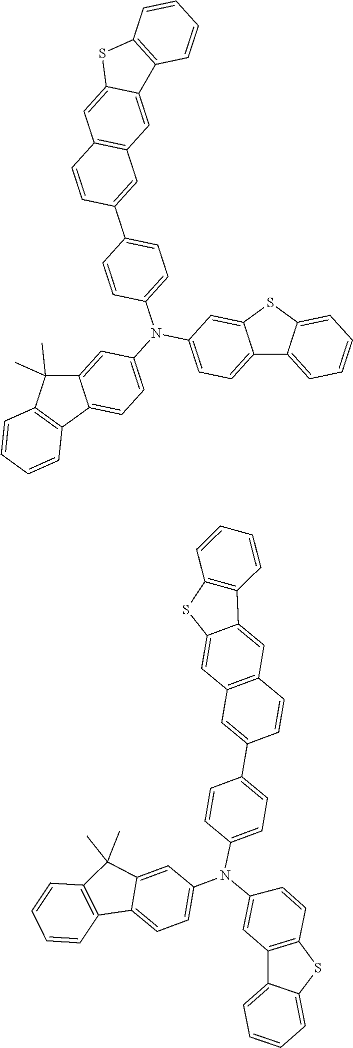 [SMILES: CC1(C)c2ccccc2-c2ccc(N(c3ccc(-c4ccc5cc6sc7ccccc7c6cc5c4)cc3)c3ccc4c(c3)sc3ccccc34)cc21 ; CC1(C)c2ccccc2-c2ccc(N(c3ccc(-c4ccc5cc6c(cc5c4)sc4ccccc46)cc3)c3ccc4sc5ccccc5c4c3)cc21]

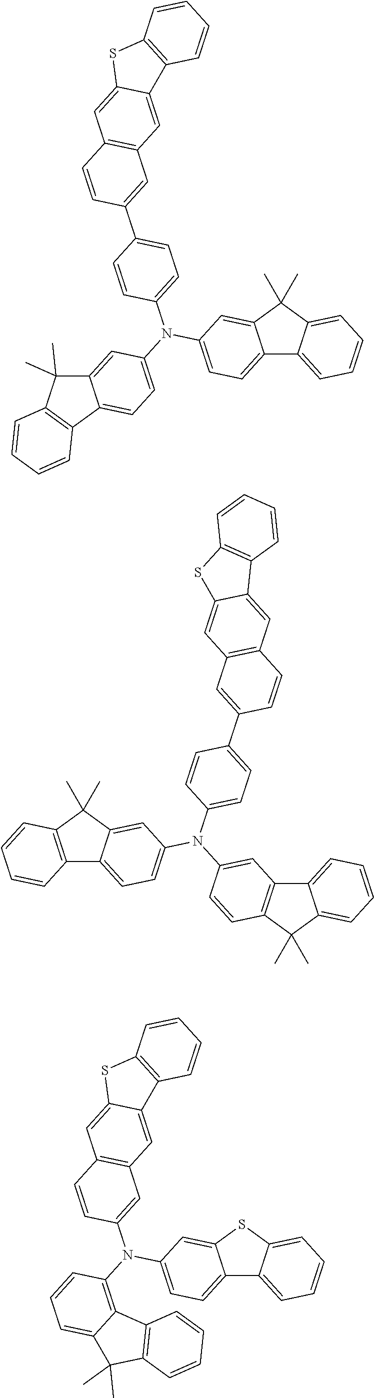 [SMILES: CC1(C)c2ccccc2-c2ccc(N(c3ccc(-c4ccc5cc6sc7ccccc7c6cc5c4)cc3)c3ccc4c(c3)C(C)(C)c3ccccc3-4)cc21 ; CC1(C)c2ccccc2-c2cc(N(c3ccc(-c4ccc5cc6c(cc5c4)sc4ccccc46)cc3)c3ccc4c(c3)C(C)(C)c3ccccc3-4)ccc21 ; CC1(C)c2ccccc2-c2c(N(c3ccc4cc5sc6ccccc6c5cc4c3)c3ccc4c(c3)sc3ccccc34)cccc21]

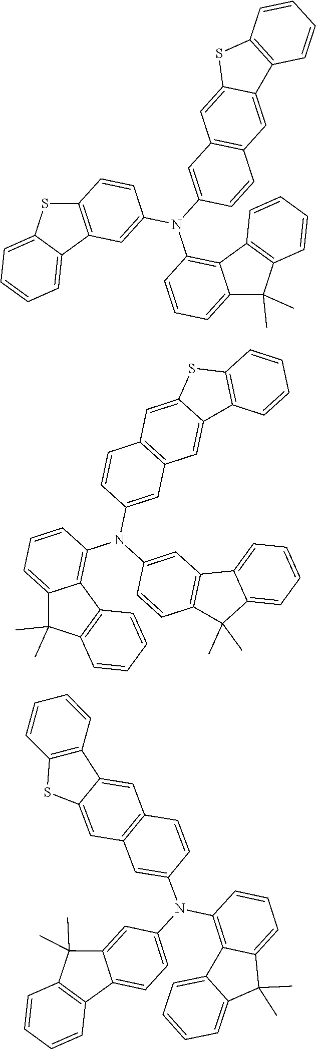

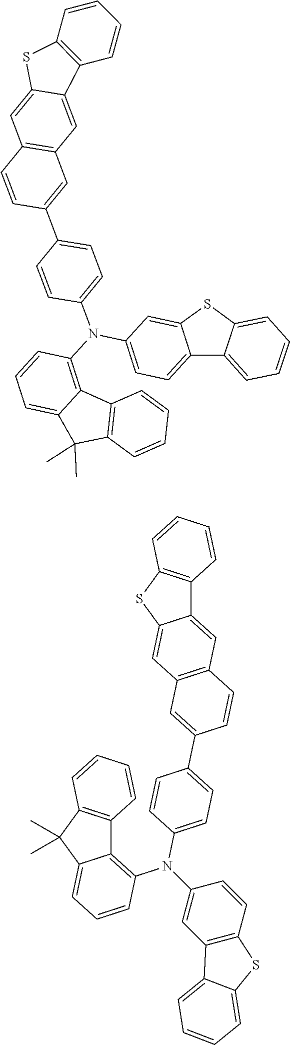 [SMILES: CC1(C)c2ccccc2-c2c(N(c3ccc(-c4ccc5cc6sc7ccccc7c6cc5c4)cc3)c3ccc4c(c3)sc3ccccc34)cccc21 ; CC1(C)c2ccccc2-c2c(N(c3ccc(-c4ccc5cc6c(cc5c4)sc4ccccc46)cc3)c3ccc4sc5ccccc5c4c3)cccc21]

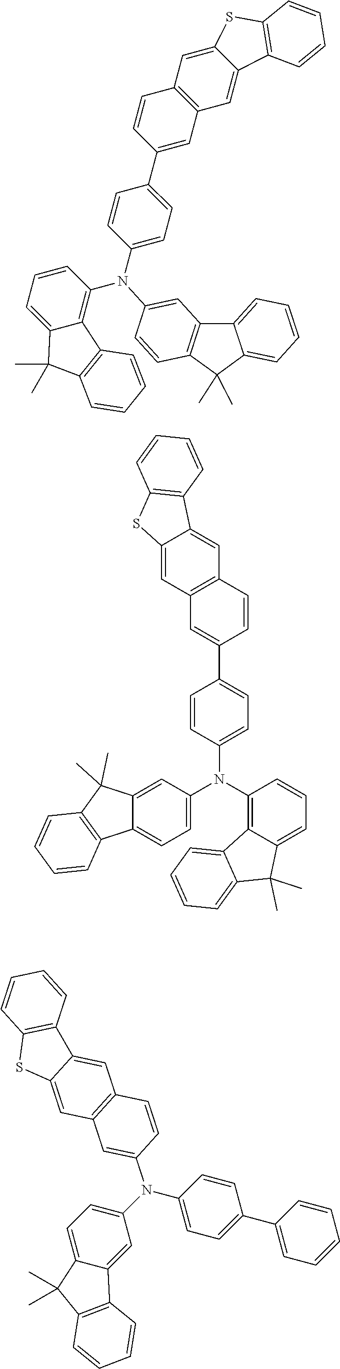

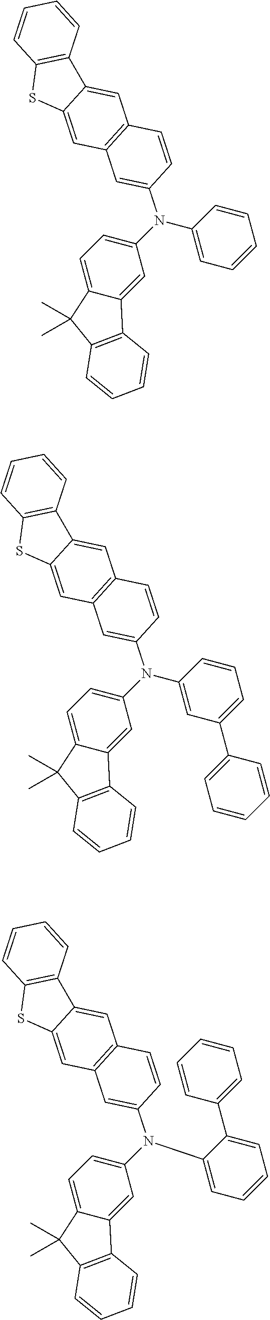 [SMILES: CC1(C)c2ccccc2-c2cc(N(c3ccccc3)c3ccc4cc5c(cc4c3)sc3ccccc35)ccc21 ; CC1(C)c2ccccc2-c2cc(N(c3cccc(-c4ccccc4)c3)c3ccc4cc5c(cc4c3)sc3ccccc35)ccc21 ; CC1(C)c2ccccc2-c2cc(N(c3ccc4cc5c(cc4c3)sc3ccccc35)c3ccccc3-c3ccccc3)ccc21]

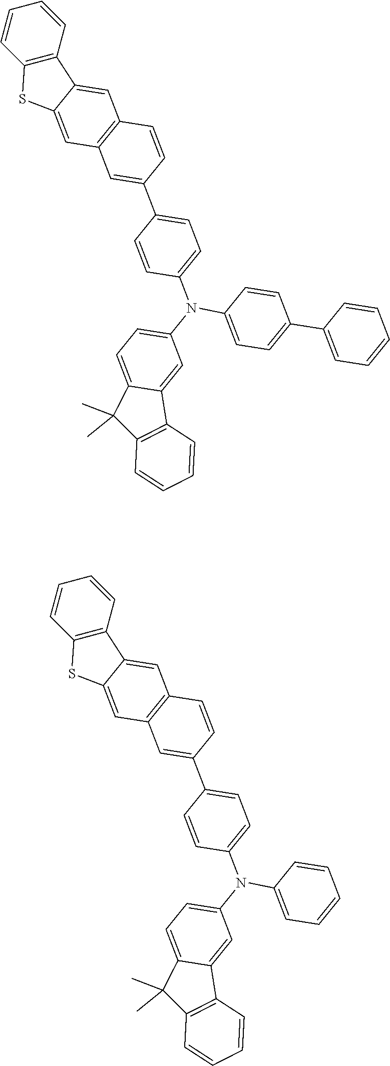 [SMILES: CC1(C)c2ccccc2-c2cc(N(c3ccc(-c4ccccc4)cc3)c3ccc(-c4ccc5cc6c(cc5c4)sc4ccccc46)cc3)ccc21 ; CC1(C)c2ccccc2-c2cc(N(c3ccccc3)c3ccc(-c4ccc5cc6c(cc5c4)sc4ccccc46)cc3)ccc21]

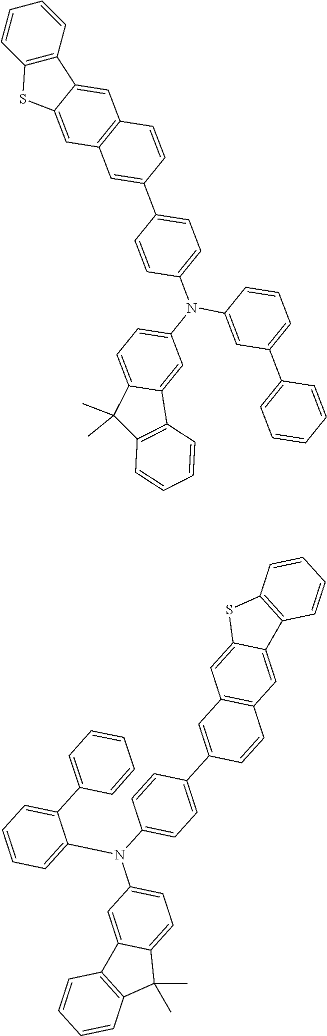 [SMILES: CC1(C)c2ccccc2-c2cc(N(c3ccc(-c4ccc5cc6c(cc5c4)sc4ccccc46)cc3)c3cccc(-c4ccccc4)c3)ccc21 ; CC1(C)c2ccccc2-c2cc(N(c3ccc(-c4ccc5cc6c(cc5c4)sc4ccccc46)cc3)c3ccccc3-c3ccccc3)ccc21]

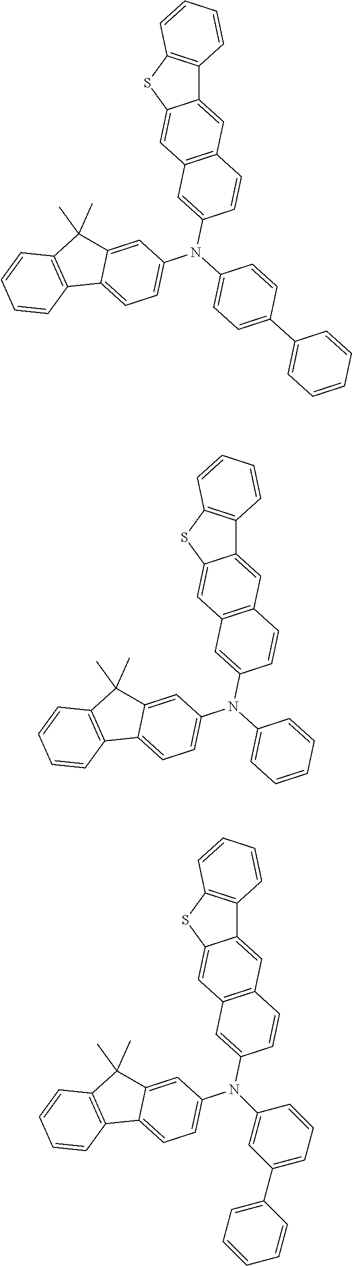 [SMILES: CC1(C)c2ccccc2-c2ccc(N(c3ccc(-c4ccccc4)cc3)c3ccc4cc5c(cc4c3)sc3ccccc35)cc21 ; CC1(C)c2ccccc2-c2ccc(N(c3ccccc3)c3ccc4cc5c(cc4c3)sc3ccccc35)cc21 ; CC1(C)c2ccccc2-c2ccc(N(c3cccc(-c4ccccc4)c3)c3ccc4cc5c(cc4c3)sc3ccccc35)cc21]

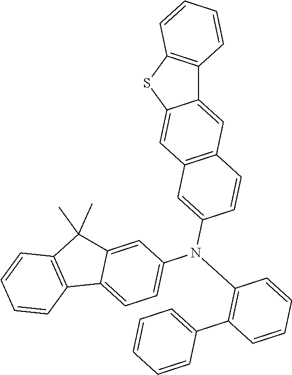 [SMILES: CC1(C)c2ccccc2-c2ccc(N(c3ccc4cc5c(cc4c3)sc3ccccc35)c3ccccc3-c3ccccc3)cc21]

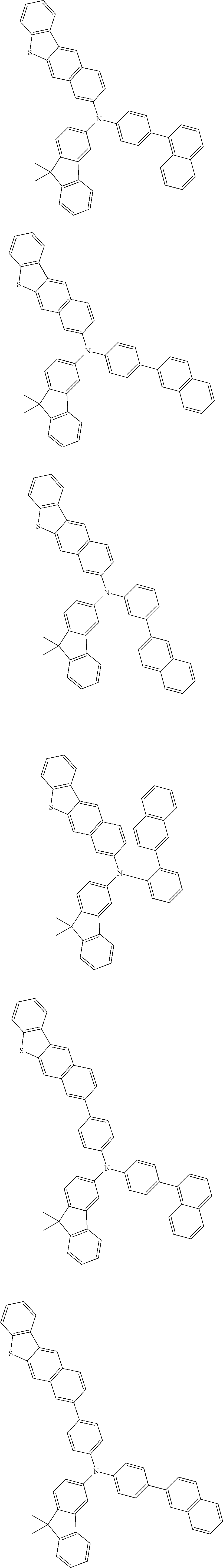

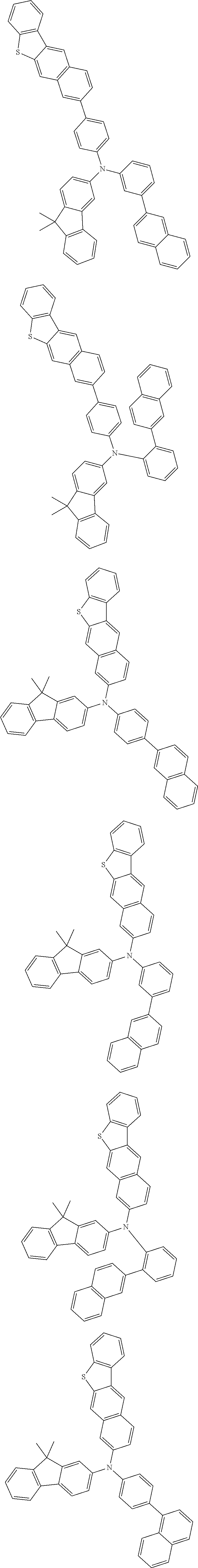

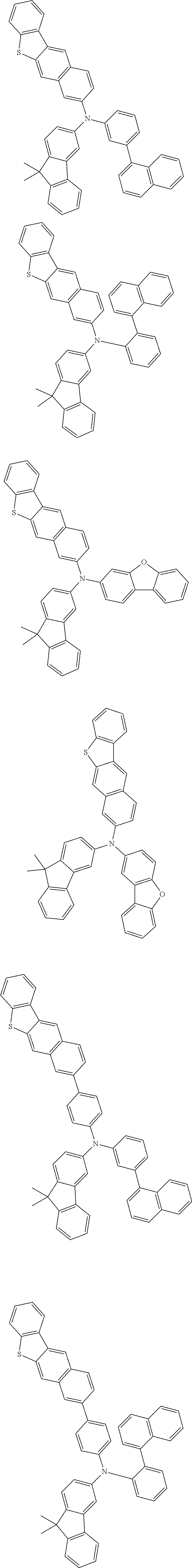

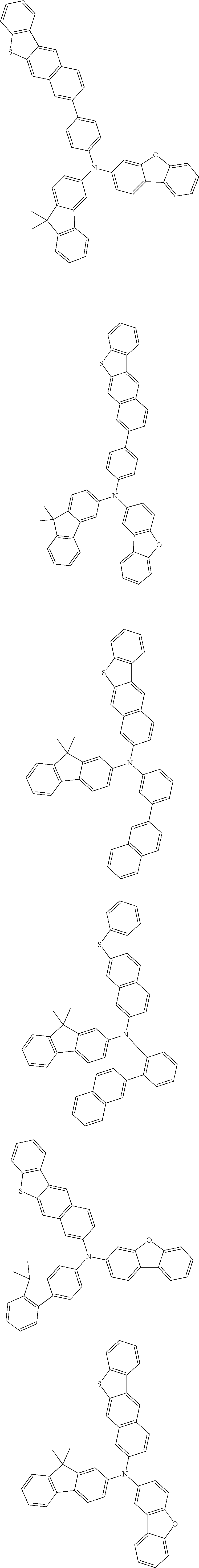

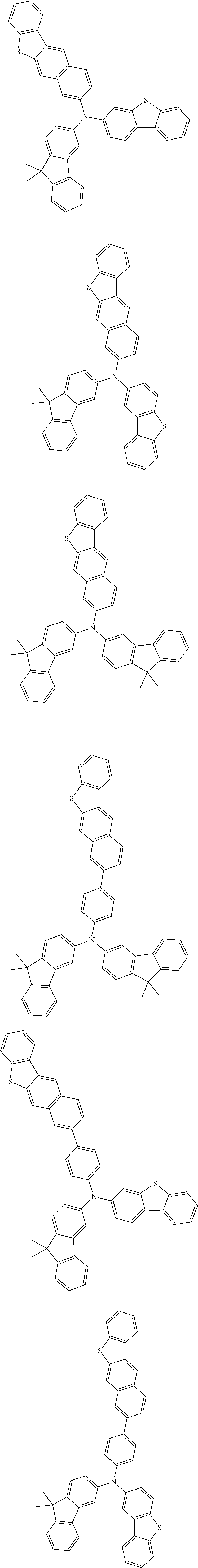

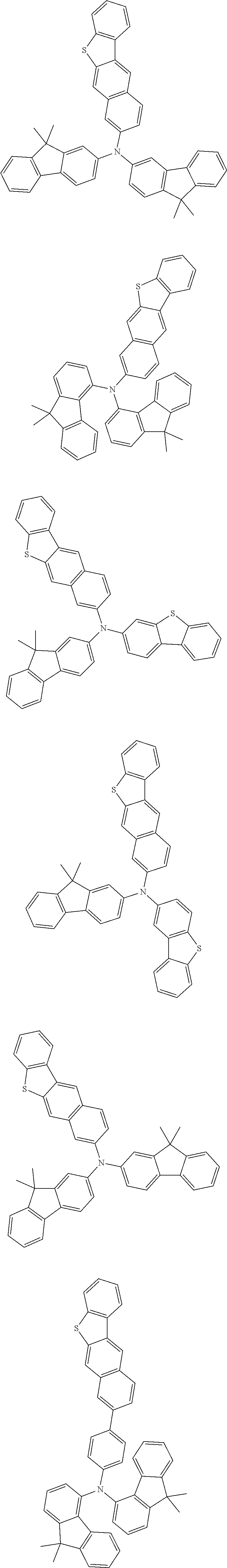

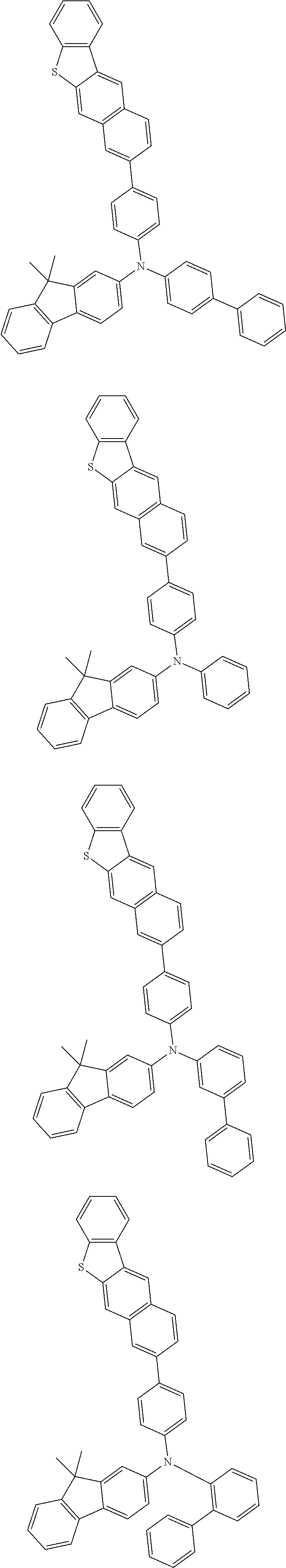 [SMILES: CC1(C)c2ccccc2-c2ccc(N(c3ccc(-c4ccccc4)cc3)c3ccc(-c4ccc5cc6c(cc5c4)sc4ccccc46)cc3)cc21 ; CC1(C)c2ccccc2-c2ccc(N(c3ccccc3)c3ccc(-c4ccc5cc6c(cc5c4)sc4ccccc46)cc3)cc21 ; CC1(C)c2ccccc2-c2ccc(N(c3ccc(-c4ccc5cc6c(cc5c4)sc4ccccc46)cc3)c3cccc(-c4ccccc4)c3)cc21 ; CC1(C)c2ccccc2-c2ccc(N(c3ccc(-c4ccc5cc6c(cc5c4)sc4ccccc46)cc3)c3ccccc3-c3ccccc3)cc21]

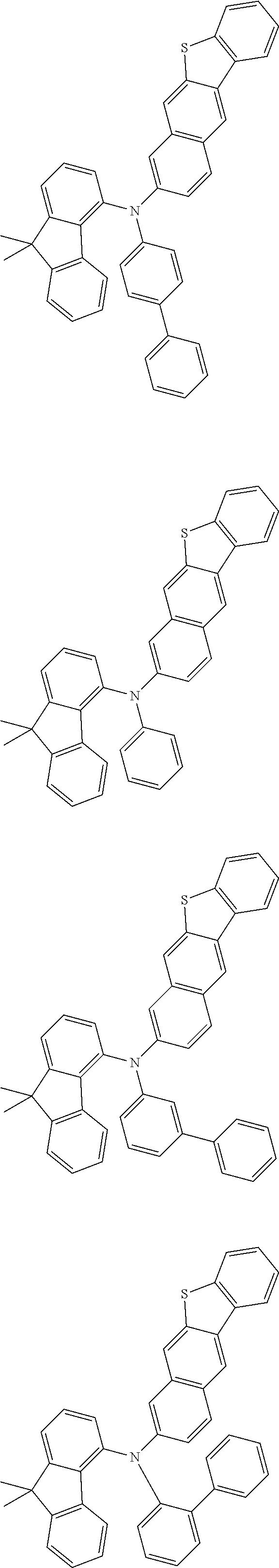 [SMILES: CC1(C)c2ccccc2-c2c(N(c3ccc(-c4ccccc4)cc3)c3ccc4cc5c(cc4c3)sc3ccccc35)cccc21 ; CC1(C)c2ccccc2-c2c(N(c3ccccc3)c3ccc4cc5c(cc4c3)sc3ccccc35)cccc21 ; CC1(C)c2ccccc2-c2c(N(c3cccc(-c4ccccc4)c3)c3ccc4cc5c(cc4c3)sc3ccccc35)cccc21 ; CC1(C)c2ccccc2-c2c(N(c3ccc4cc5c(cc4c3)sc3ccccc35)c3ccccc3-c3ccccc3)cccc21]

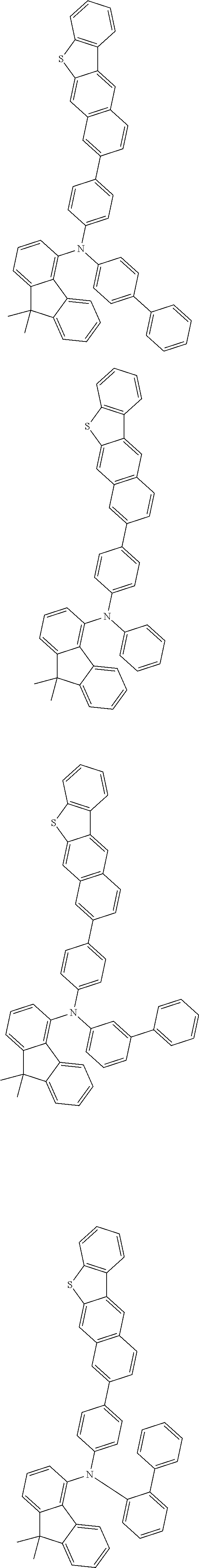 [SMILES: CC1(C)c2ccccc2-c2c(N(c3ccc(-c4ccccc4)cc3)c3ccc(-c4ccc5cc6c(cc5c4)sc4ccccc46)cc3)cccc21 ; CC1(C)c2ccccc2-c2c(N(c3ccccc3)c3ccc(-c4ccc5cc6c(cc5c4)sc4ccccc46)cc3)cccc21 ; CC1(C)c2ccccc2-c2c(N(c3ccc(-c4ccc5cc6c(cc5c4)sc4ccccc46)cc3)c3cccc(-c4ccccc4)c3)cccc21 ; CC1(C)c2ccccc2-c2c(N(c3ccc(-c4ccc5cc6c(cc5c4)sc4ccccc46)cc3)c3ccccc3-c3ccccc3)cccc21]

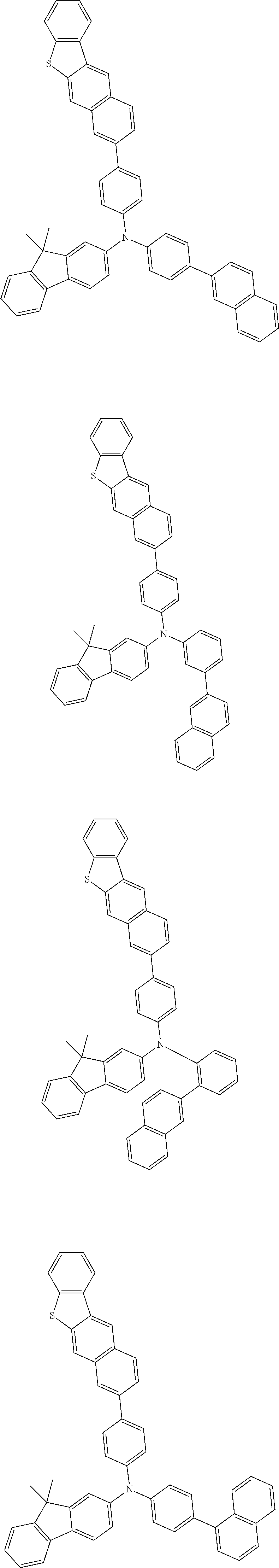 [SMILES: CC1(C)c2ccccc2-c2ccc(N(c3ccc(-c4ccc5ccccc5c4)cc3)c3ccc(-c4ccc5cc6c(cc5c4)sc4ccccc46)cc3)cc21 ; CC1(C)c2ccccc2-c2ccc(N(c3ccc(-c4ccc5cc6c(cc5c4)sc4ccccc46)cc3)c3cccc(-c4ccc5ccccc5c4)c3)cc21 ; CC1(C)c2ccccc2-c2ccc(N(c3ccc(-c4ccc5cc6c(cc5c4)sc4ccccc46)cc3)c3ccccc3-c3ccc4ccccc4c3)cc21 ; CC1(C)c2ccccc2-c2ccc(N(c3ccc(-c4ccc5cc6c(cc5c4)sc4ccccc46)cc3)c3ccc(-c4cccc5ccccc45)cc3)cc21]

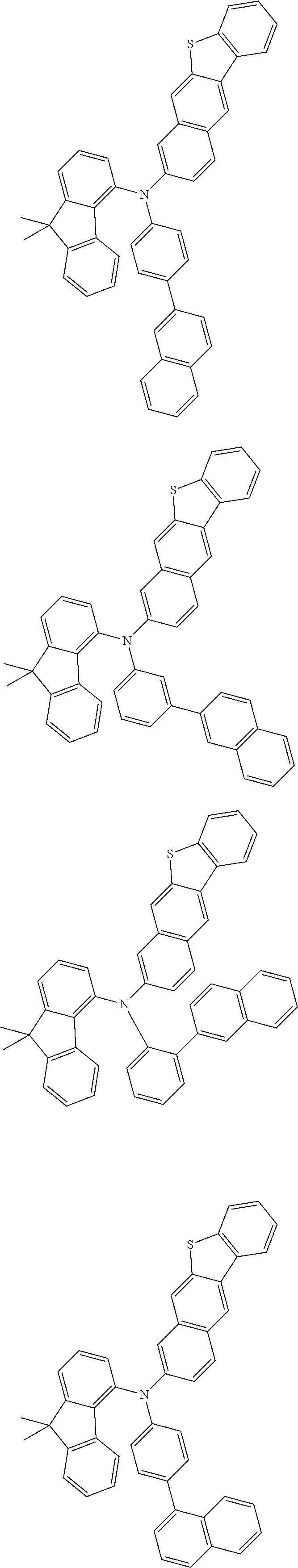

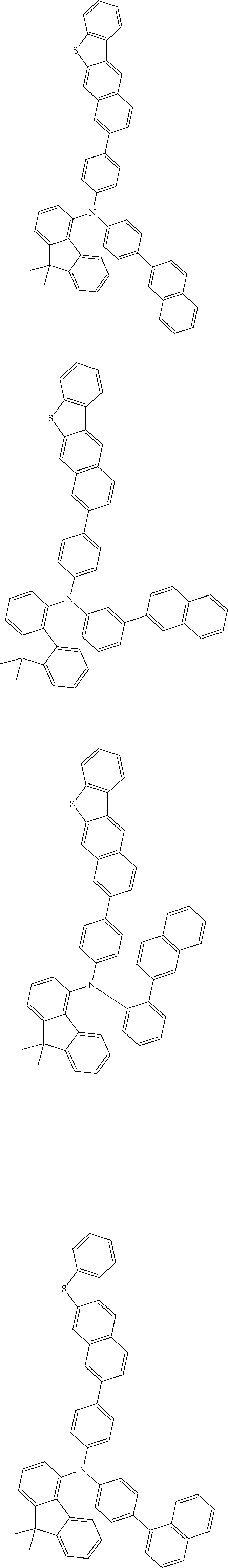 [SMILES: CC1(C)c2ccccc2-c2c(N(c3ccc(-c4ccc5ccccc5c4)cc3)c3ccc(-c4ccc5cc6c(cc5c4)sc4ccccc46)cc3)cccc21 ; CC1(C)c2ccccc2-c2c(N(c3ccc(-c4ccc5cc6c(cc5c4)sc4ccccc46)cc3)c3cccc(-c4ccc5ccccc5c4)c3)cccc21 ; CC1(C)c2ccccc2-c2c(N(c3ccc(-c4ccc5cc6c(cc5c4)sc4ccccc46)cc3)c3ccccc3-c3ccc4ccccc4c3)cccc21 ; CC1(C)c2ccccc2-c2c(N(c3ccc(-c4ccc5cc6c(cc5c4)sc4ccccc46)cc3)c3ccc(-c4cccc5ccccc45)cc3)cccc21]

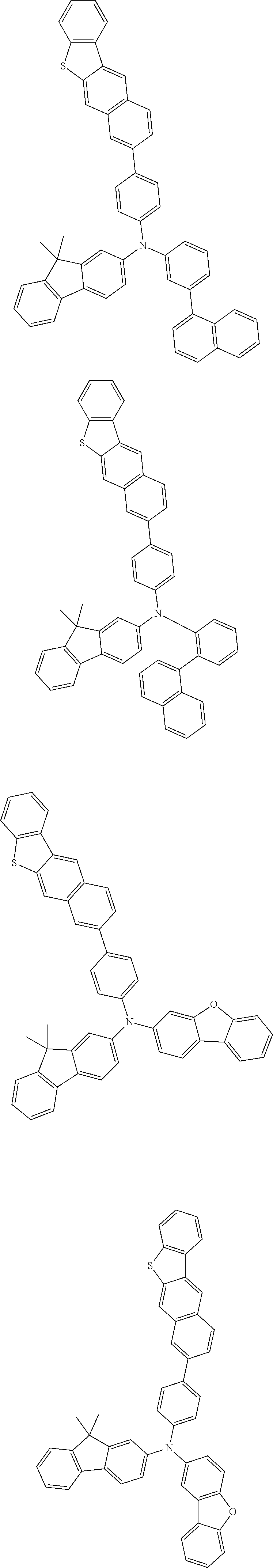 [SMILES: CC1(C)c2ccccc2-c2ccc(N(c3ccc(-c4ccc5cc6c(cc5c4)sc4ccccc46)cc3)c3cccc(-c4cccc5ccccc45)c3)cc21 ; CC1(C)c2ccccc2-c2ccc(N(c3ccc(-c4ccc5cc6c(cc5c4)sc4ccccc46)cc3)c3ccccc3-c3cccc4ccccc34)cc21 ; CC1(C)c2ccccc2-c2ccc(N(c3ccc(-c4ccc5cc6c(cc5c4)sc4ccccc46)cc3)c3ccc4c(c3)oc3ccccc34)cc21 ; CC1(C)c2ccccc2-c2ccc(N(c3ccc(-c4ccc5cc6c(cc5c4)sc4ccccc46)cc3)c3ccc4oc5ccccc5c4c3)cc21]

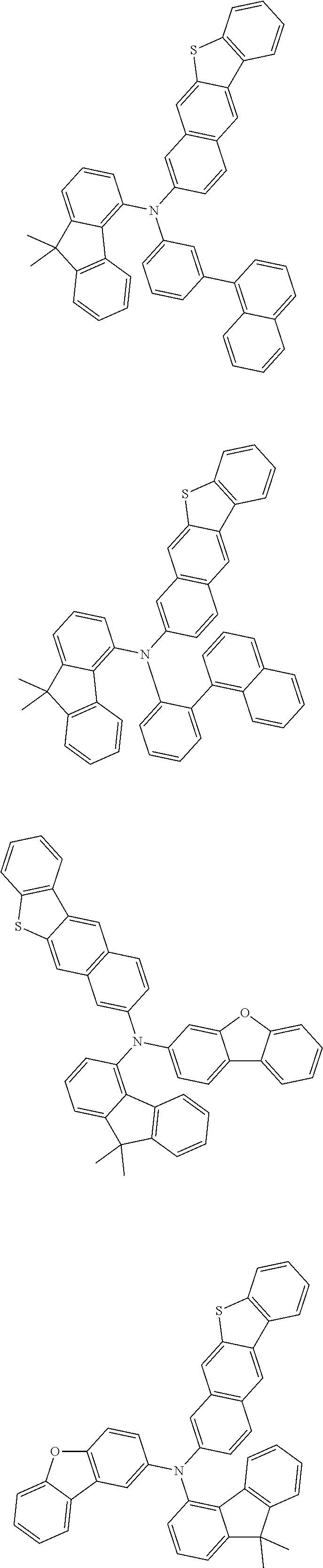 [SMILES: CC1(C)c2ccccc2-c2c(N(c3cccc(-c4cccc5ccccc45)c3)c3ccc4cc5c(cc4c3)sc3ccccc35)cccc21 ; CC1(C)c2ccccc2-c2c(N(c3ccc4cc5c(cc4c3)sc3ccccc35)c3ccccc3-c3cccc4ccccc34)cccc21 ; CC1(C)c2ccccc2-c2c(N(c3ccc4cc5c(cc4c3)sc3ccccc35)c3ccc4c(c3)oc3ccccc34)cccc21 ; CC1(C)c2ccccc2-c2c(N(c3ccc4cc5c(cc4c3)sc3ccccc35)c3ccc4oc5ccccc5c4c3)cccc21]

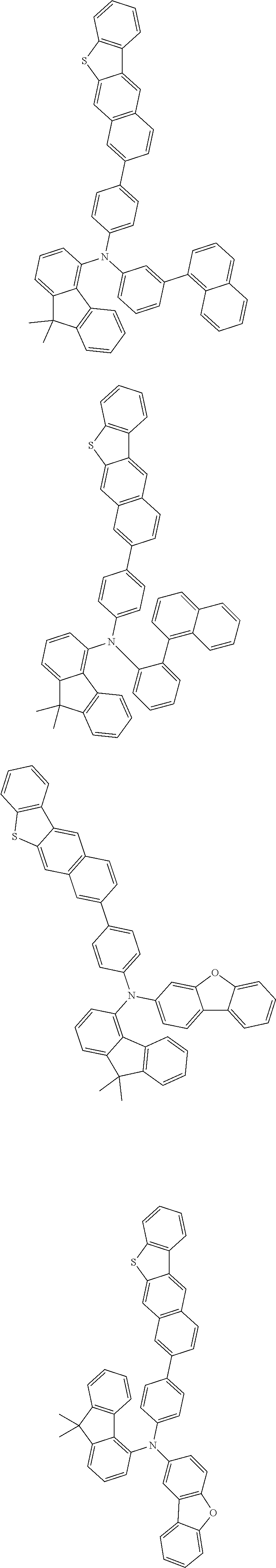

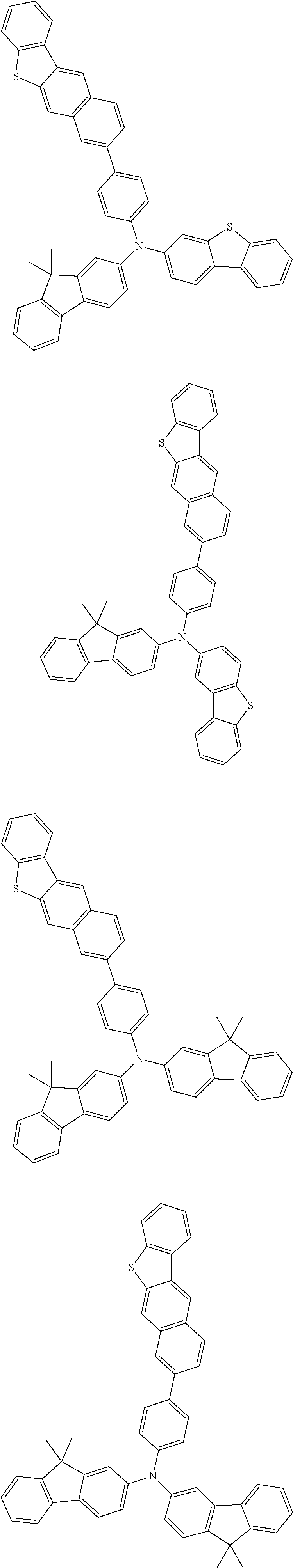 [SMILES: CC1(C)c2ccccc2-c2ccc(N(c3ccc(-c4ccc5cc6c(cc5c4)sc4ccccc46)cc3)c3ccc4c(c3)sc3ccccc34)cc21 ; CC1(C)c2ccccc2-c2ccc(N(c3ccc(-c4ccc5cc6c(cc5c4)sc4ccccc46)cc3)c3ccc4sc5ccccc5c4c3)cc21 ; CC1(C)c2ccccc2-c2ccc(N(c3ccc(-c4ccc5cc6c(cc5c4)sc4ccccc46)cc3)c3ccc4c(c3)C(C)(C)c3ccccc3-4)cc21 ; CC1(C)c2ccccc2-c2cc(N(c3ccc(-c4ccc5cc6c(cc5c4)sc4ccccc46)cc3)c3ccc4c(c3)C(C)(C)c3ccccc3-4)ccc21]

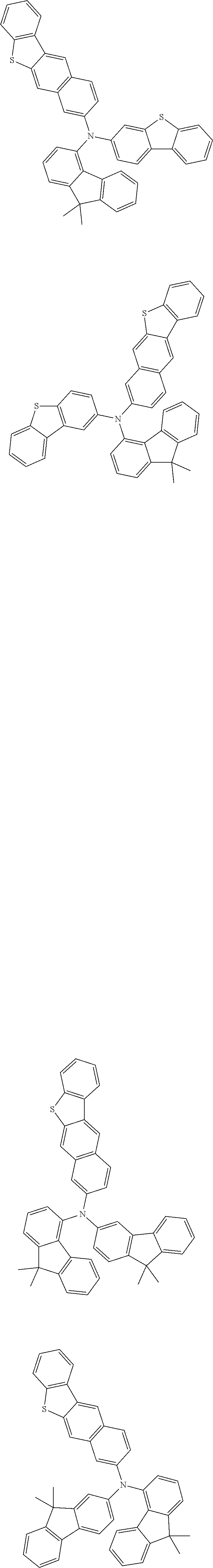 [SMILES: CC1(C)c2ccccc2-c2c(N(c3ccc4cc5c(cc4c3)sc3ccccc35)c3ccc4c(c3)sc3ccccc34)cccc21 ; CC1(C)c2ccccc2-c2c(N(c3ccc4cc5c(cc4c3)sc3ccccc35)c3ccc4sc5ccccc5c4c3)cccc21 ; CC1(C)c2ccccc2-c2cc(N(c3ccc4cc5c(cc4c3)sc3ccccc35)c3cccc4c3-c3ccccc3C4(C)C)ccc21 ; CC1(C)c2ccccc2-c2ccc(N(c3ccc4cc5c(cc4c3)sc3ccccc35)c3cccc4c3-c3ccccc3C4(C)C)cc21]

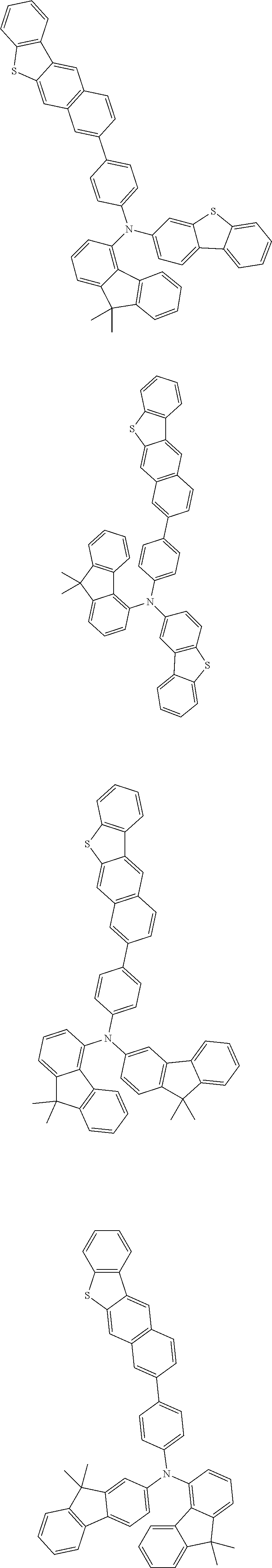 [SMILES: CC1(C)c2ccccc2-c2c(N(c3ccc(-c4ccc5cc6c(cc5c4)sc4ccccc46)cc3)c3ccc4c(c3)sc3ccccc34)cccc21 ; CC1(C)c2ccccc2-c2c(N(c3ccc(-c4ccc5cc6c(cc5c4)sc4ccccc46)cc3)c3ccc4sc5ccccc5c4c3)cccc21 ; CC1(C)c2ccccc2-c2cc(N(c3ccc(-c4ccc5cc6c(cc5c4)sc4ccccc46)cc3)c3cccc4c3-c3ccccc3C4(C)C)ccc21 ; CC1(C)c2ccccc2-c2ccc(N(c3ccc(-c4ccc5cc6c(cc5c4)sc4ccccc46)cc3)c3cccc4c3-c3ccccc3C4(C)C)cc21]

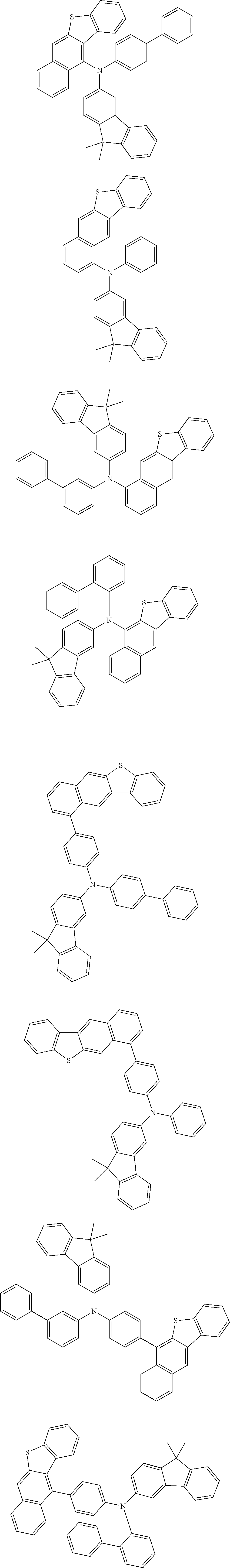

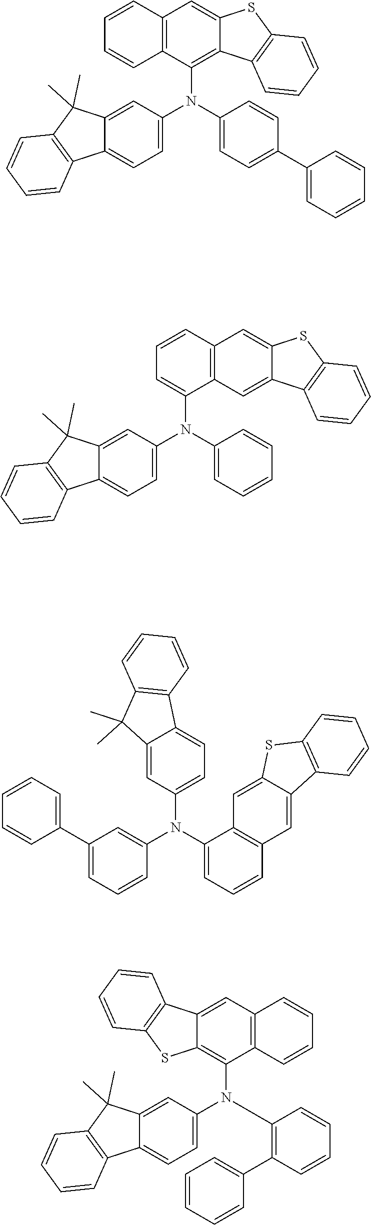 [SMILES: CC1(C)c2ccccc2-c2ccc(N(c3ccc(-c4ccccc4)cc3)c3c4ccccc4cc4sc5ccccc5c34)cc21 ; CC1(C)c2ccccc2-c2ccc(N(c3ccccc3)c3cccc4cc5sc6ccccc6c5cc34)cc21 ; CC1(C)c2ccccc2-c2ccc(N(c3cccc(-c4ccccc4)c3)c3cccc4cc5c(cc34)sc3ccccc35)cc21 ; CC1(C)c2ccccc2-c2ccc(N(c3ccccc3-c3ccccc3)c3c4ccccc4cc4c3sc3ccccc34)cc21]

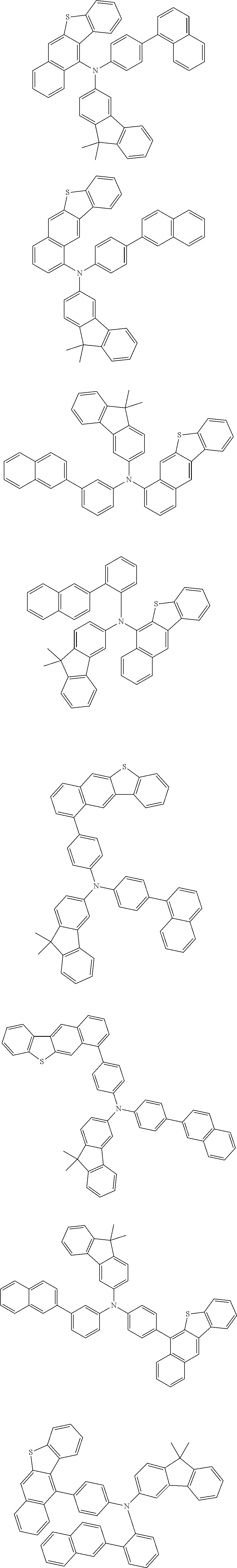

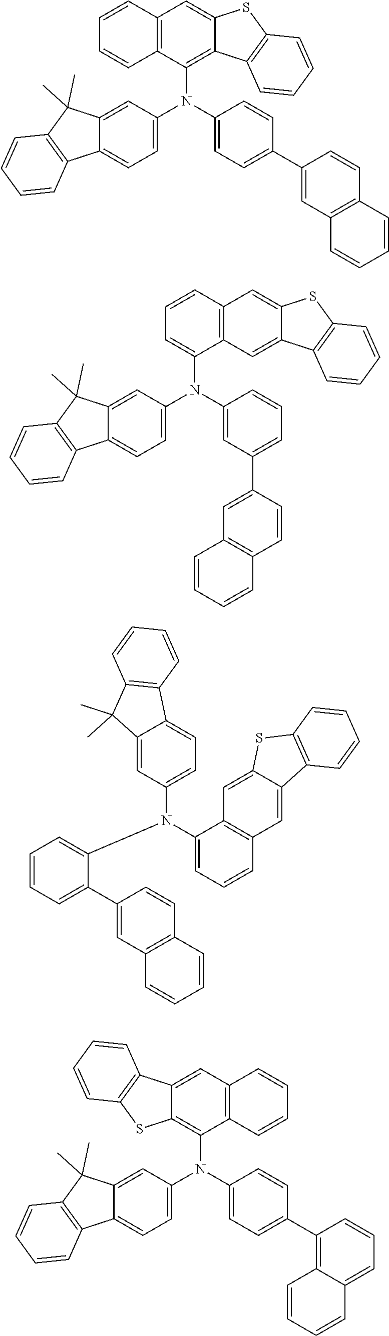 [SMILES: CC1(C)c2ccccc2-c2ccc(N(c3ccc(-c4ccc5ccccc5c4)cc3)c3c4ccccc4cc4sc5ccccc5c34)cc21 ; CC1(C)c2ccccc2-c2ccc(N(c3cccc(-c4ccc5ccccc5c4)c3)c3cccc4cc5sc6ccccc6c5cc34)cc21 ; CC1(C)c2ccccc2-c2ccc(N(c3ccccc3-c3ccc4ccccc4c3)c3cccc4cc5c(cc34)sc3ccccc35)cc21 ; CC1(C)c2ccccc2-c2ccc(N(c3ccc(-c4cccc5ccccc45)cc3)c3c4ccccc4cc4c3sc3ccccc34)cc21]

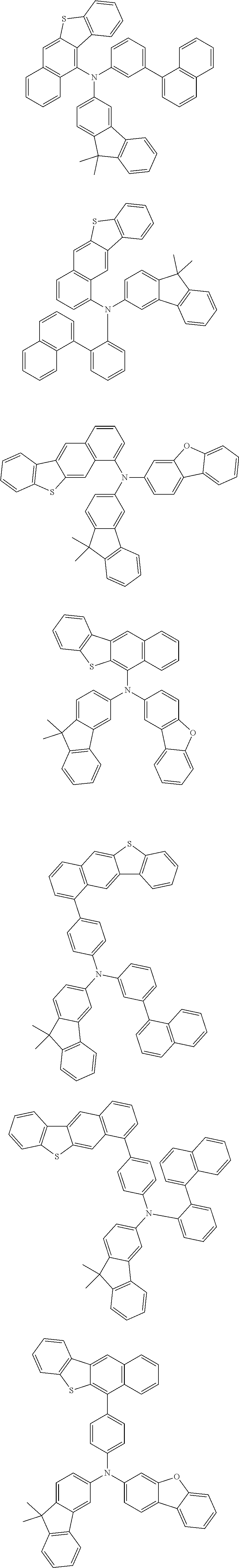

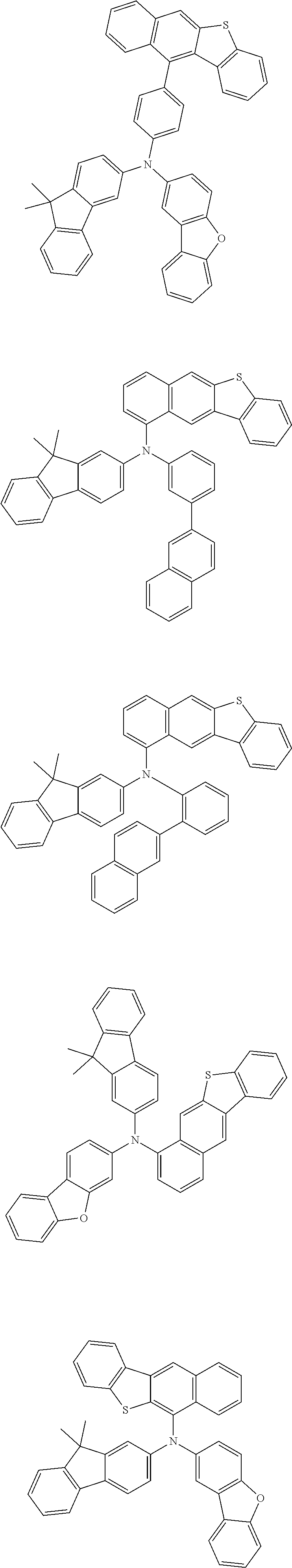 [SMILES: CC1(C)c2ccccc2-c2cc(N(c3ccc(-c4c5ccccc5cc5sc6ccccc6c45)cc3)c3ccc4oc5ccccc5c4c3)ccc21 ; CC1(C)c2ccccc2-c2ccc(N(c3cccc(-c4ccc5ccccc5c4)c3)c3cccc4cc5sc6ccccc6c5cc34)cc21 ; CC1(C)c2ccccc2-c2ccc(N(c3ccccc3-c3ccc4ccccc4c3)c3cccc4cc5sc6ccccc6c5cc34)cc21 ; CC1(C)c2ccccc2-c2ccc(N(c3ccc4c(c3)oc3ccccc34)c3cccc4cc5c(cc34)sc3ccccc35)cc21 ; CC1(C)c2ccccc2-c2ccc(N(c3ccc4oc5ccccc5c4c3)c3c4ccccc4cc4c3sc3ccccc34)cc21]

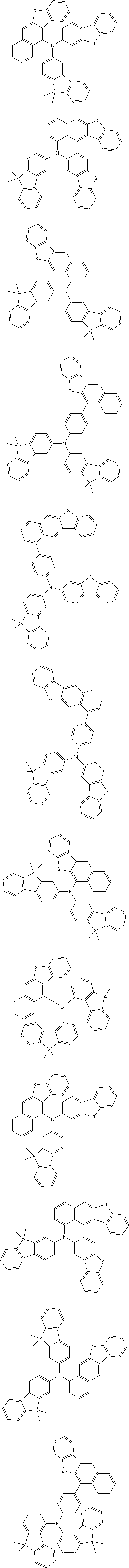

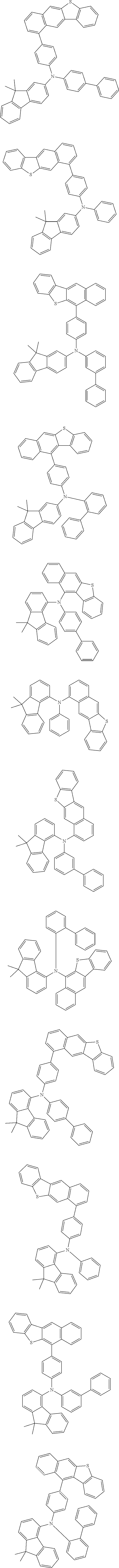

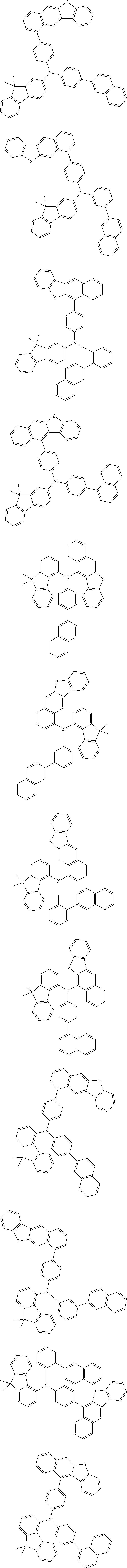

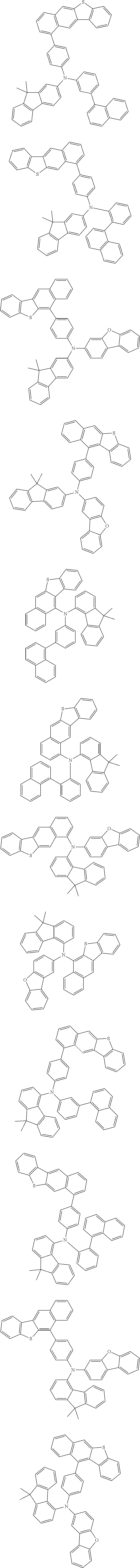

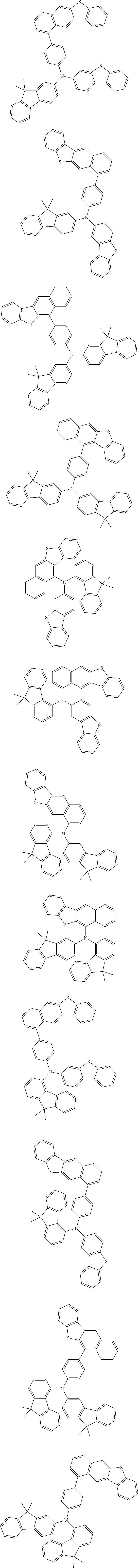

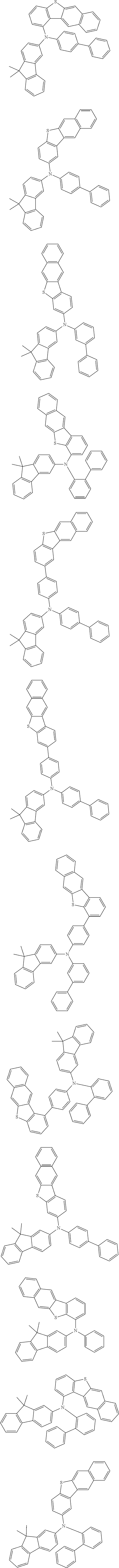

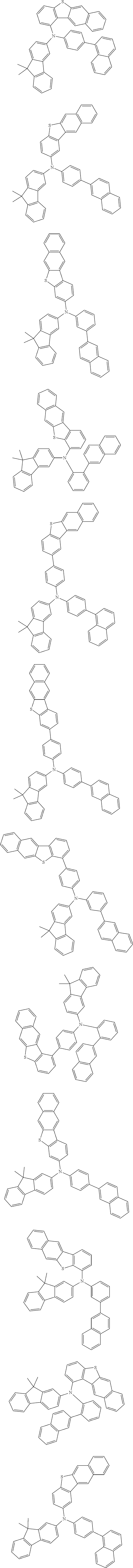

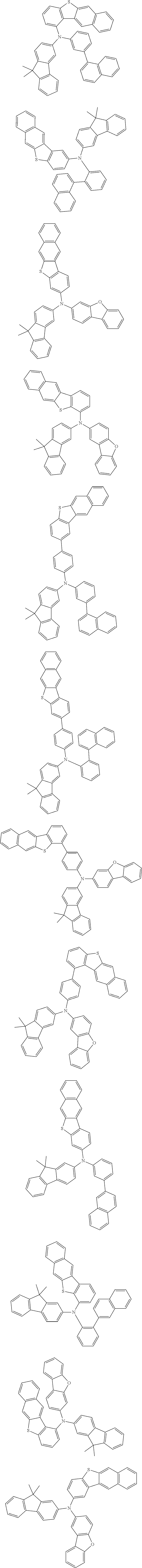

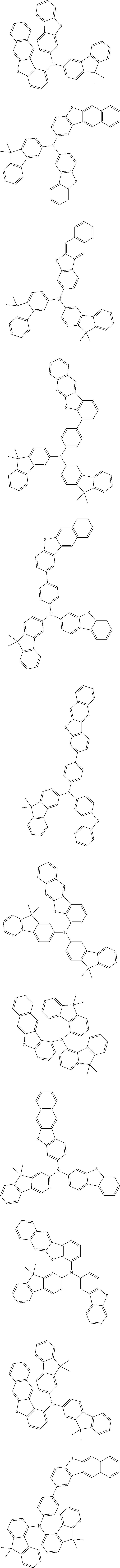

[chemical structure]

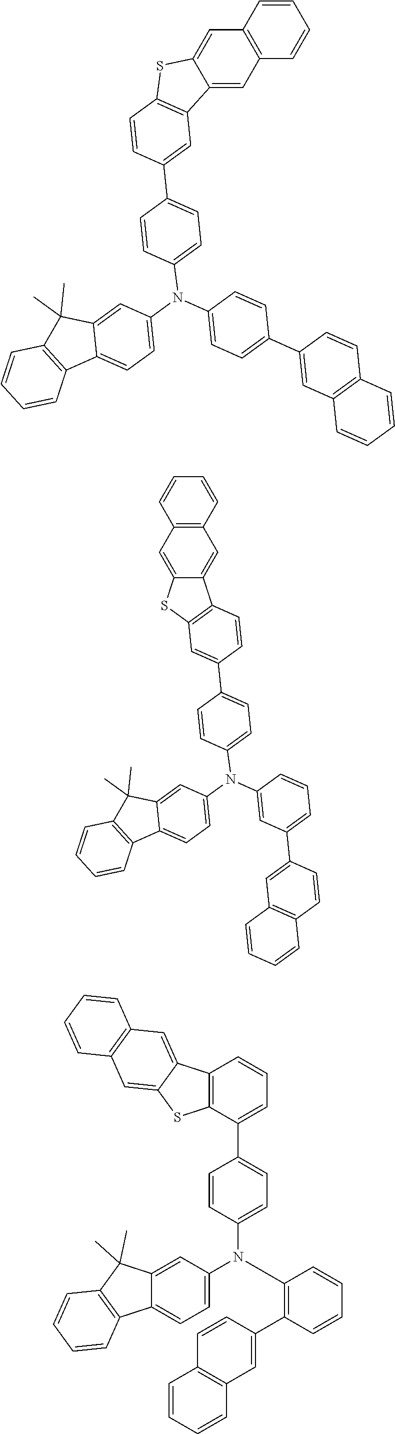 [SMILES: CC1(C)c2ccccc2-c2ccc(N(c3ccc(-c4ccc5ccccc5c4)cc3)c3ccc(-c4ccc5sc6cc7ccccc7cc6c5c4)cc3)cc21 ; CC1(C)c2ccccc2-c2ccc(N(c3ccc(-c4ccc5c(c4)sc4cc6ccccc6cc45)cc3)c3cccc(-c4ccc5ccccc5c4)c3)cc21 ; CC1(C)c2ccccc2-c2ccc(N(c3ccc(-c4cccc5c4sc4cc6ccccc6cc45)cc3)c3ccccc3-c3ccc4ccccc4c3)cc21]

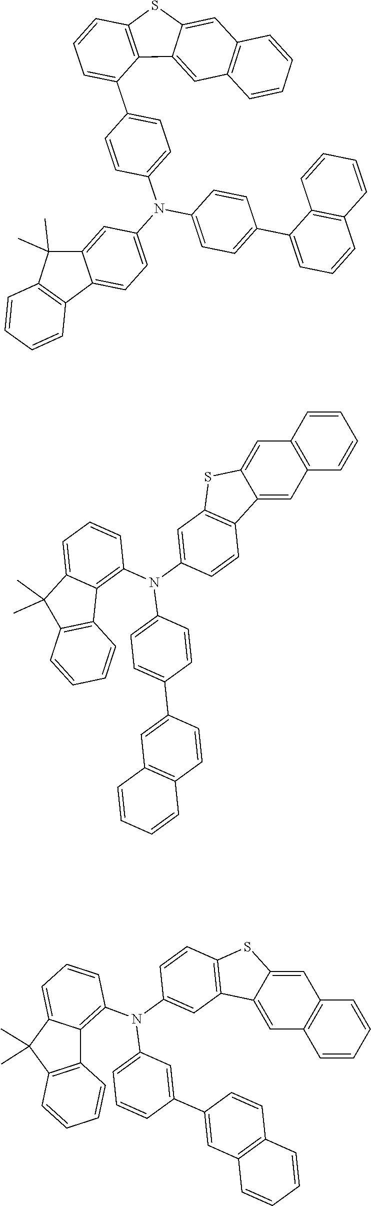 [SMILES: CC1(C)c2ccccc2-c2ccc(N(c3ccc(-c4cccc5ccccc45)cc3)c3ccc(-c4cccc5sc6cc7ccccc7cc6c45)cc3)cc21 ; CC1(C)c2ccccc2-c2c(N(c3ccc(-c4ccc5ccccc5c4)cc3)c3ccc4c(c3)sc3cc5ccccc5cc34)cccc21 ; CC1(C)c2ccccc2-c2c(N(c3cccc(-c4ccc5ccccc5c4)c3)c3ccc4sc5cc6ccccc6cc5c4c3)cccc21]

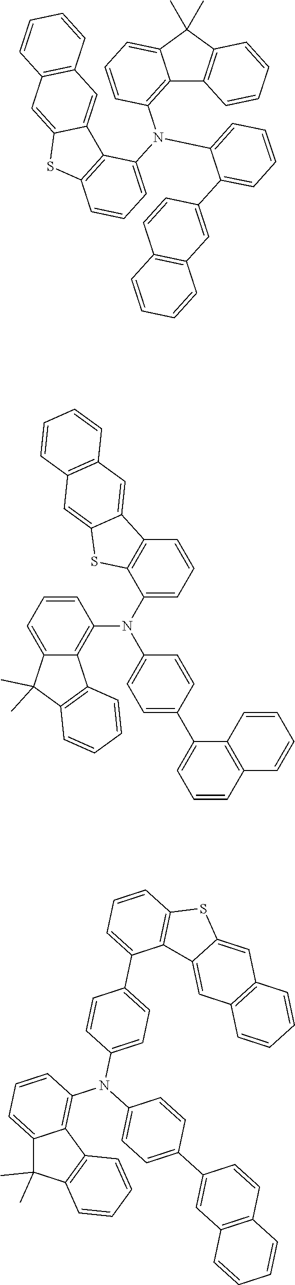 [SMILES: CC1(C)c2ccccc2-c2c(N(c3ccccc3-c3ccc4ccccc4c3)c3cccc4sc5cc6ccccc6cc5c34)cccc21 ; CC1(C)c2ccccc2-c2c(N(c3ccc(-c4cccc5ccccc45)cc3)c3cccc4c3sc3cc5ccccc5cc34)cccc21 ; CC1(C)c2ccccc2-c2c(N(c3ccc(-c4ccc5ccccc5c4)cc3)c3ccc(-c4cccc5sc6cc7ccccc7cc6c45)cc3)cccc21]

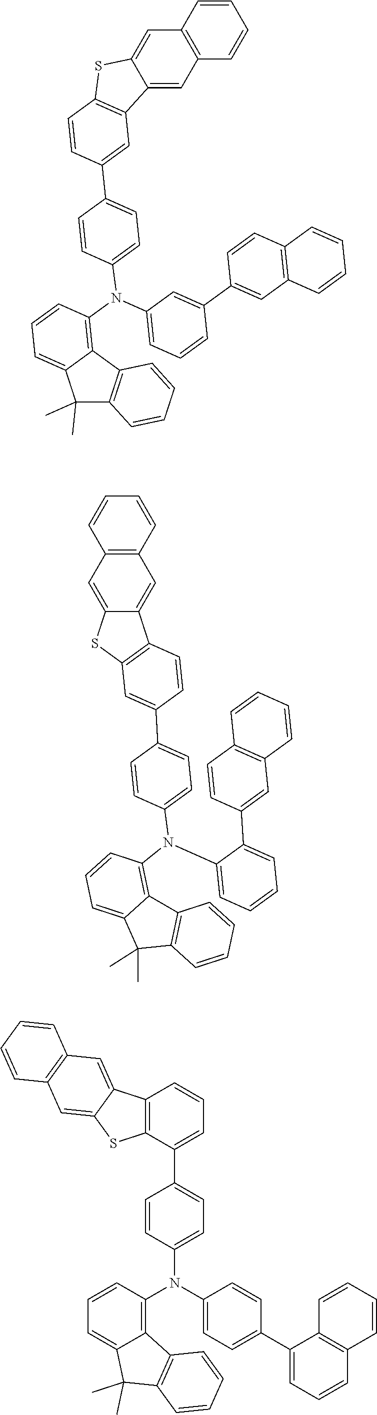 [SMILES: CC1(C)c2ccccc2-c2c(N(c3ccc(-c4ccc5sc6cc7ccccc7cc6c5c4)cc3)c3cccc(-c4ccc5ccccc5c4)c3)cccc21 ; CC1(C)c2ccccc2-c2c(N(c3ccc(-c4ccc5c(c4)sc4cc6ccccc6cc45)cc3)c3ccccc3-c3ccc4ccccc4c3)cccc21 ; CC1(C)c2ccccc2-c2c(N(c3ccc(-c4cccc5ccccc45)cc3)c3ccc(-c4cccc5c4sc4cc6ccccc6cc45)cc3)cccc21]

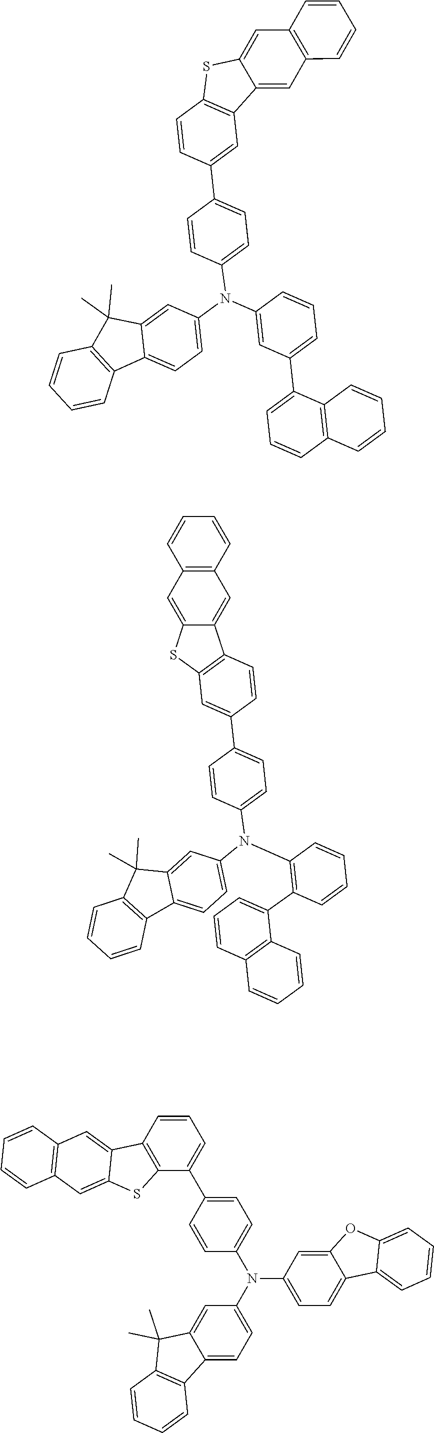 [SMILES: CC1(C)c2ccccc2-c2ccc(N(c3ccc(-c4ccc5sc6cc7ccccc7cc6c5c4)cc3)c3cccc(-c4cccc5ccccc45)c3)cc21 ; CC1(C)c2ccccc2-c2ccc(N(c3ccc(-c4ccc5c(c4)sc4cc6ccccc6cc45)cc3)c3ccccc3-c3cccc4ccccc34)cc21 ; CC1(C)c2ccccc2-c2ccc(N(c3ccc(-c4cccc5c4sc4cc6ccccc6cc45)cc3)c3ccc4c(c3)oc3ccccc34)cc21]

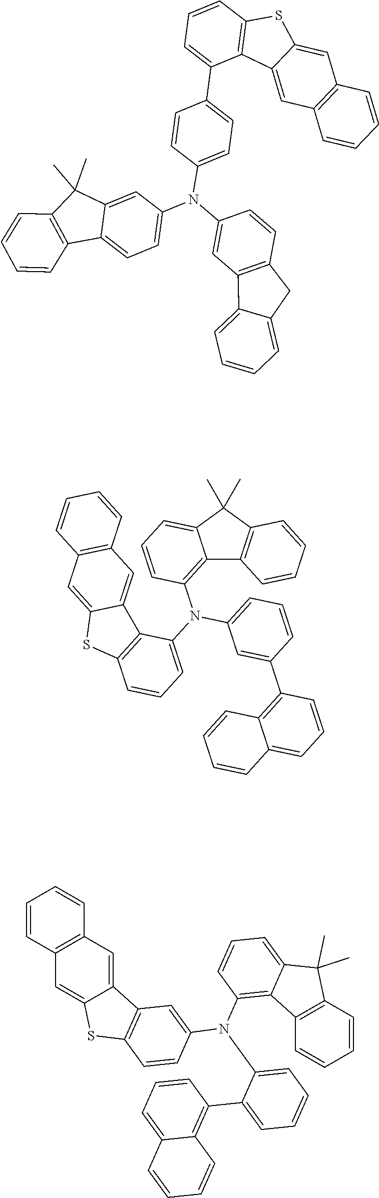 [SMILES: CC1(C)c2ccccc2-c2ccc(N(c3ccc(-c4cccc5sc6cc7ccccc7cc6c45)cc3)c3ccc4c(c3)-c3ccccc3C4)cc21 ; CC1(C)c2ccccc2-c2c(N(c3cccc(-c4cccc5ccccc45)c3)c3cccc4sc5cc6ccccc6cc5c34)cccc21 ; CC1(C)c2ccccc2-c2c(N(c3ccc4sc5cc6ccccc6cc5c4c3)c3ccccc3-c3cccc4ccccc34)cccc21]

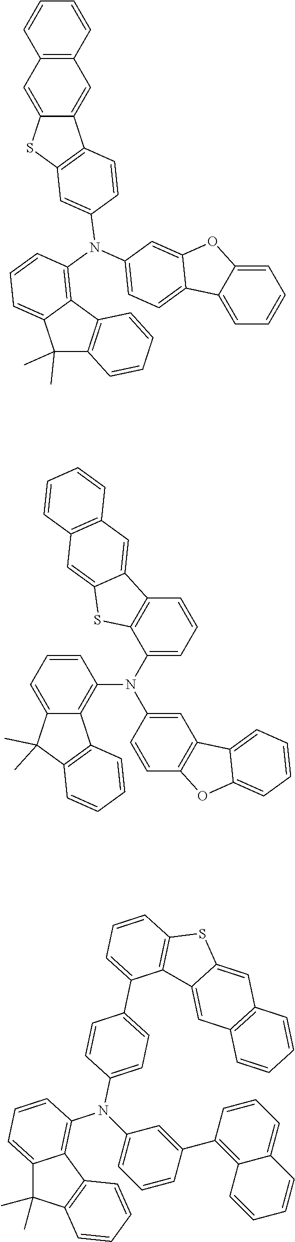 [SMILES: CC1(C)c2ccccc2-c2c(N(c3ccc4c(c3)oc3ccccc34)c3ccc4c(c3)sc3cc5ccccc5cc34)cccc21 ; CC1(C)c2ccccc2-c2c(N(c3ccc4oc5ccccc5c4c3)c3cccc4c3sc3cc5ccccc5cc34)cccc21 ; CC1(C)c2ccccc2-c2c(N(c3ccc(-c4cccc5sc6cc7ccccc7cc6c45)cc3)c3cccc(-c4cccc5ccccc45)c3)cccc21]

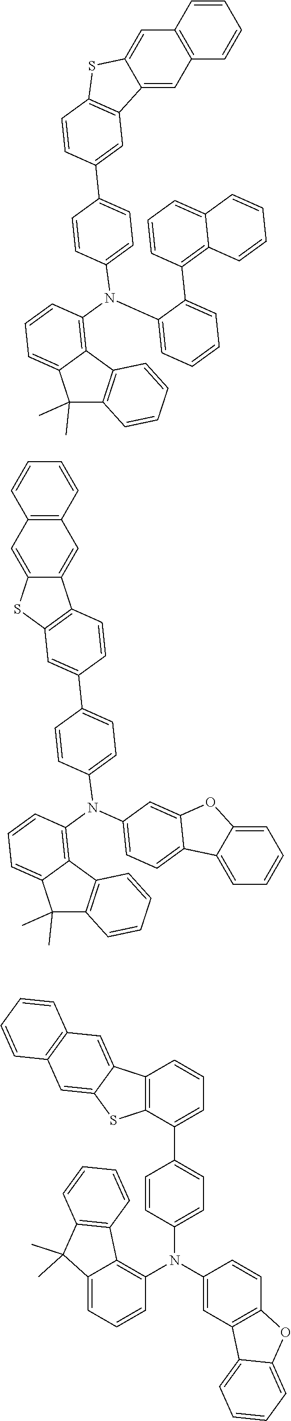

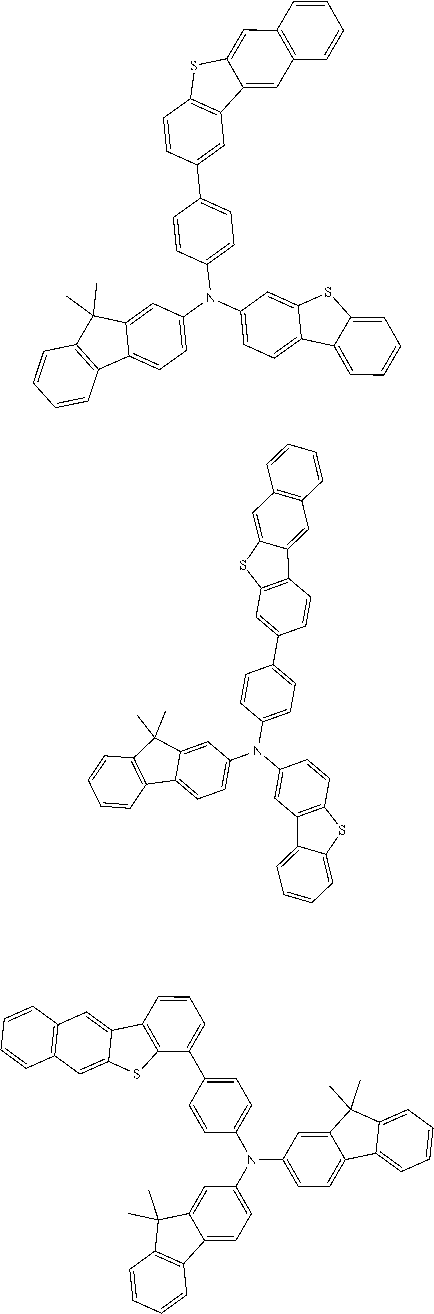 [SMILES: CC1(C)c2ccccc2-c2ccc(N(c3ccc(-c4ccc5sc6cc7ccccc7cc6c5c4)cc3)c3ccc4c(c3)sc3ccccc34)cc21 ; CC1(C)c2ccccc2-c2ccc(N(c3ccc(-c4ccc5c(c4)sc4cc6ccccc6cc45)cc3)c3ccc4sc5ccccc5c4c3)cc21 ; CC1(C)c2ccccc2-c2ccc(N(c3ccc(-c4cccc5c4sc4cc6ccccc6cc45)cc3)c3ccc4c(c3)C(C)(C)c3ccccc3-4)cc21]

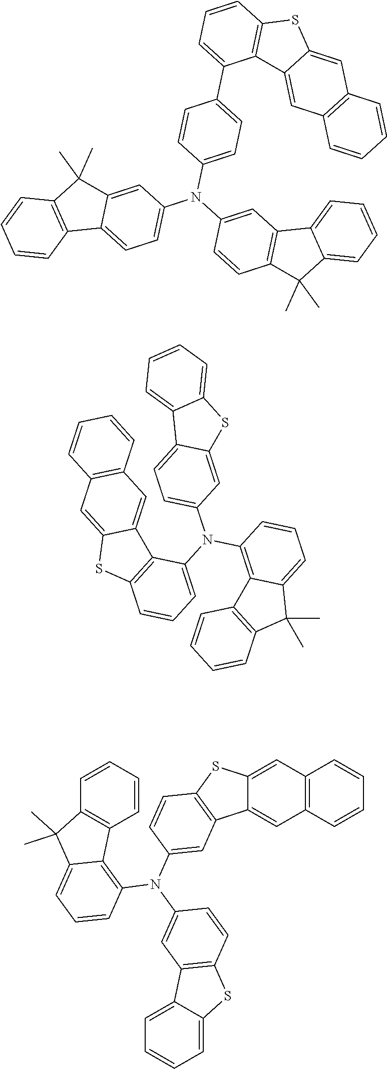

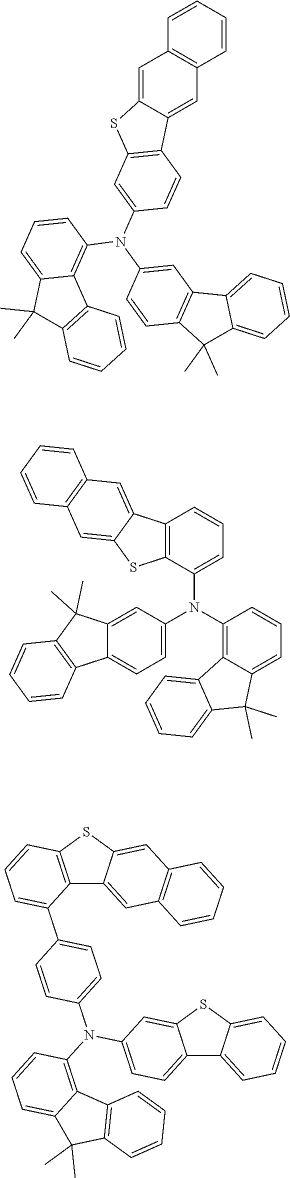 [SMILES: CC1(C)c2ccccc2-c2cc(N(c3ccc4c(c3)sc3cc5ccccc5cc34)c3cccc4c3-c3ccccc3C4(C)C)ccc21 ; CC1(C)c2ccccc2-c2ccc(N(c3cccc4c3-c3ccccc3C4(C)C)c3cccc4c3sc3cc5ccccc5cc34)cc21 ; CC1(C)c2ccccc2-c2c(N(c3ccc(-c4cccc5sc6cc7ccccc7cc6c45)cc3)c3ccc4c(c3)sc3ccccc34)cccc21]

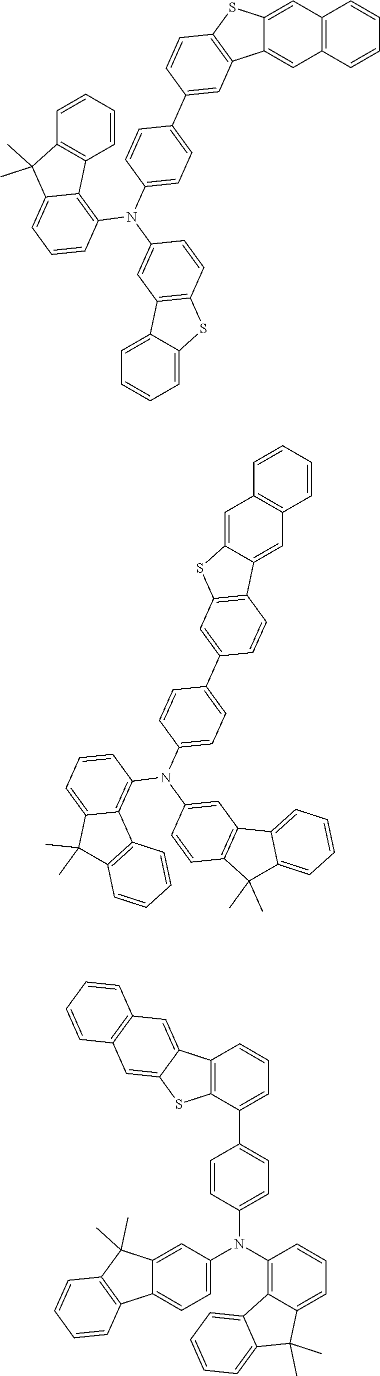 [SMILES: CC1(C)c2ccccc2-c2c(N(c3ccc(-c4ccc5sc6cc7ccccc7cc6c5c4)cc3)c3ccc4sc5ccccc5c4c3)cccc21 ; CC1(C)c2ccccc2-c2cc(N(c3ccc(-c4ccc5c(c4)sc4cc6ccccc6cc45)cc3)c3cccc4c3-c3ccccc3C4(C)C)ccc21 ; CC1(C)c2ccccc2-c2ccc(N(c3ccc(-c4cccc5c4sc4cc6ccccc6cc45)cc3)c3cccc4c3-c3ccccc3C4(C)C)cc21]

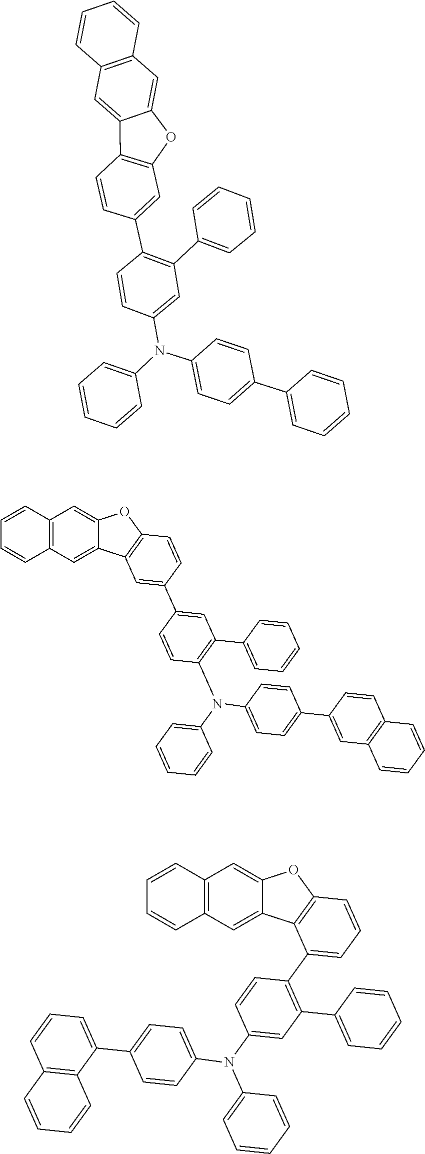 [SMILES: c1ccc(-c2ccc(N(c3ccccc3)c3ccc(-c4ccc5c(c4)oc4cc6ccccc6cc45)c(-c4ccccc4)c3)cc2)cc1 ; c1ccc(-c2cc(-c3ccc4oc5cc6ccccc6cc5c4c3)ccc2N(c2ccccc2)c2ccc(-c3ccc4ccccc4c3)cc2)cc1 ; c1ccc(-c2cc(N(c3ccccc3)c3ccc(-c4cccc5ccccc45)cc3)ccc2-c2cccc3oc4cc5ccccc5cc4c23)cc1]

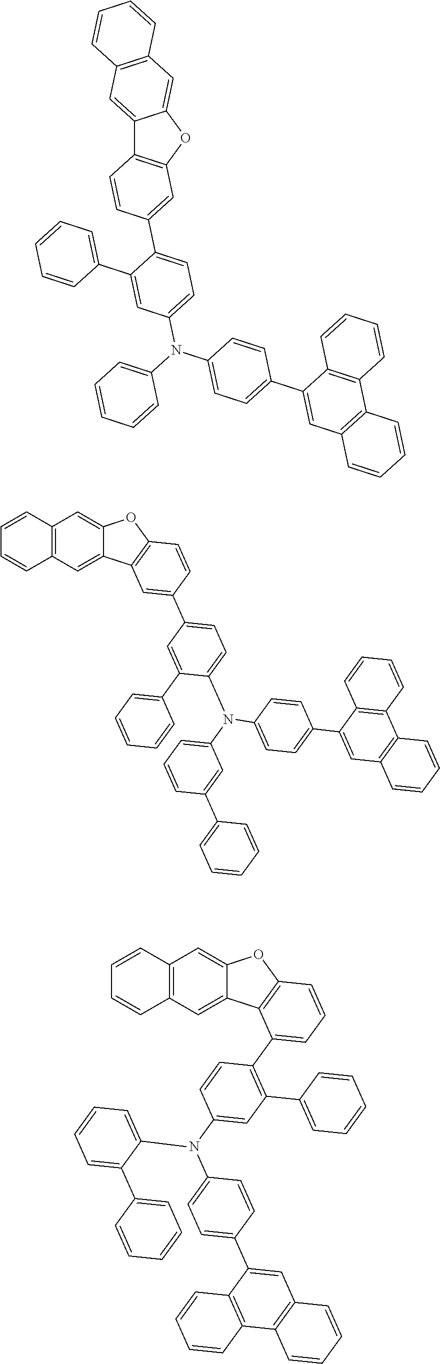 [SMILES: c1ccc(-c2cc(N(c3ccccc3)c3ccc(-c4cc5ccccc5c5ccccc45)cc3)ccc2-c2ccc3c(c2)oc2cc4ccccc4cc23)cc1 ; c1ccc(-c2cccc(N(c3ccc(-c4cc5ccccc5c5ccccc45)cc3)c3ccc(-c4ccc5oc6cc7ccccc7cc6c5c4)cc3-c3ccccc3)c2)cc1 ; c1ccc(-c2cc(N(c3ccc(-c4cc5ccccc5c5ccccc45)cc3)c3ccccc3-c3ccccc3)ccc2-c2cccc3oc4cc5ccccc5cc4c23)cc1]

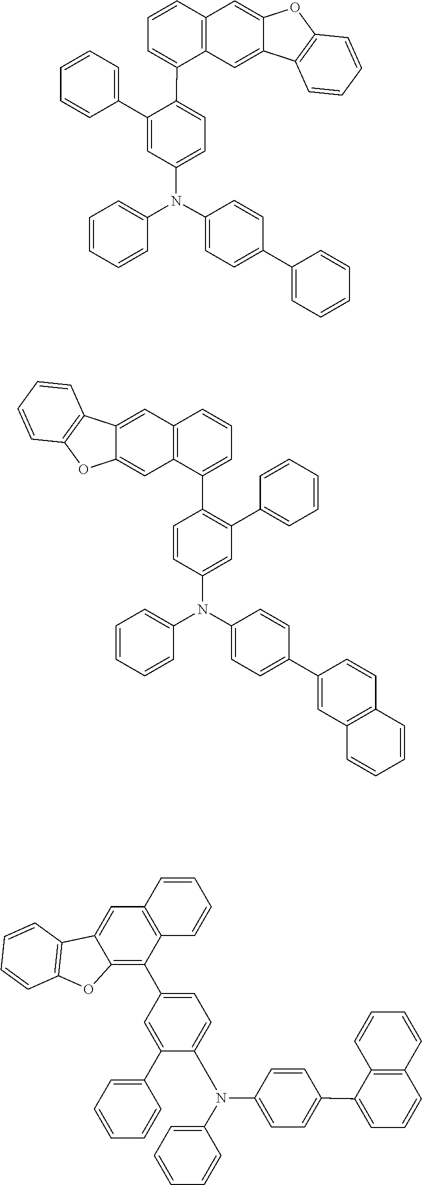 [SMILES: c1ccc(-c2ccc(N(c3ccccc3)c3ccc(-c4cccc5cc6oc7ccccc7c6cc45)c(-c4ccccc4)c3)cc2)cc1 ; c1ccc(-c2cc(N(c3ccccc3)c3ccc(-c4ccc5ccccc5c4)cc3)ccc2-c2cccc3cc4c(cc23)oc2ccccc24)cc1 ; c1ccc(-c2cc(-c3c4ccccc4cc4c3oc3ccccc34)ccc2N(c2ccccc2)c2ccc(-c3cccc4ccccc34)cc2)cc1]

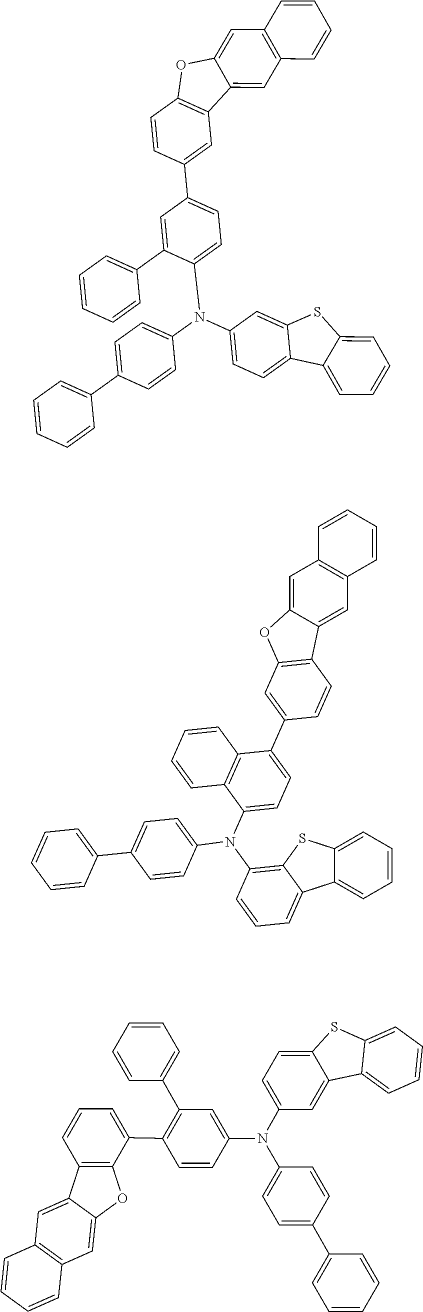 [SMILES: c1ccc(-c2ccc(N(c3ccc4c(c3)sc3ccccc34)c3ccc(-c4ccc5oc6cc7ccccc7cc6c5c4)cc3-c3ccccc3)cc2)cc1 ; c1ccc(-c2ccc(N(c3ccc(-c4ccc5c(c4)oc4cc6ccccc6cc45)c4ccccc34)c3cccc4c3sc3ccccc34)cc2)cc1 ; c1ccc(-c2ccc(N(c3ccc(-c4cccc5c4oc4cc6ccccc6cc45)c(-c4ccccc4)c3)c3ccc4sc5ccccc5c4c3)cc2)cc1]

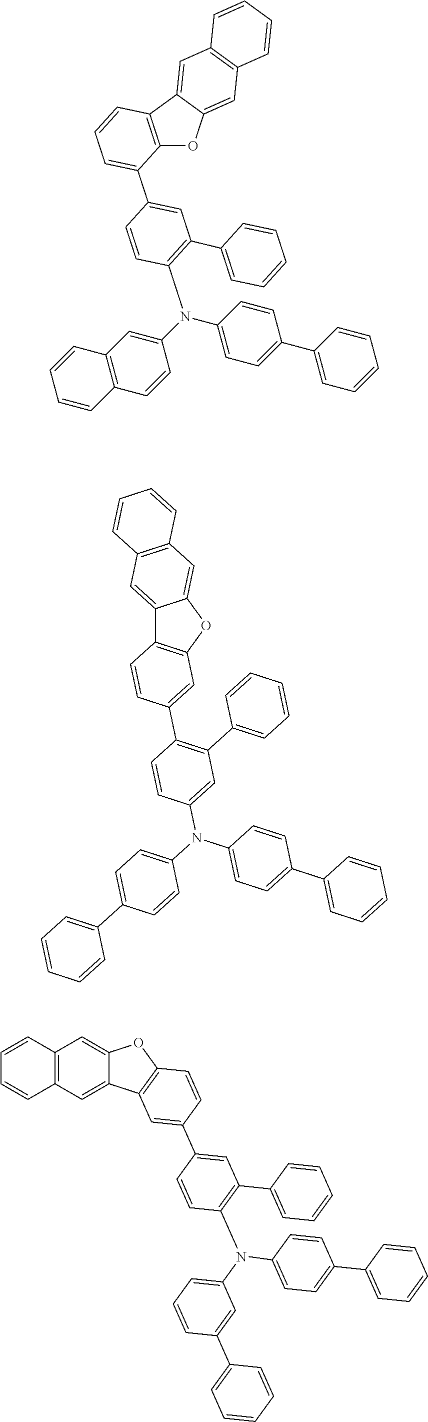 [SMILES: c1ccc(-c2ccc(N(c3ccc4ccccc4c3)c3ccc(-c4cccc5c4oc4cc6ccccc6cc45)cc3-c3ccccc3)cc2)cc1 ; c1ccc(-c2ccc(N(c3ccc(-c4ccccc4)cc3)c3ccc(-c4ccc5c(c4)oc4cc6ccccc6cc45)c(-c4ccccc4)c3)cc2)cc1 ; c1ccc(-c2ccc(N(c3cccc(-c4ccccc4)c3)c3ccc(-c4ccc5oc6cc7ccccc7cc6c5c4)cc3-c3ccccc3)cc2)cc1]

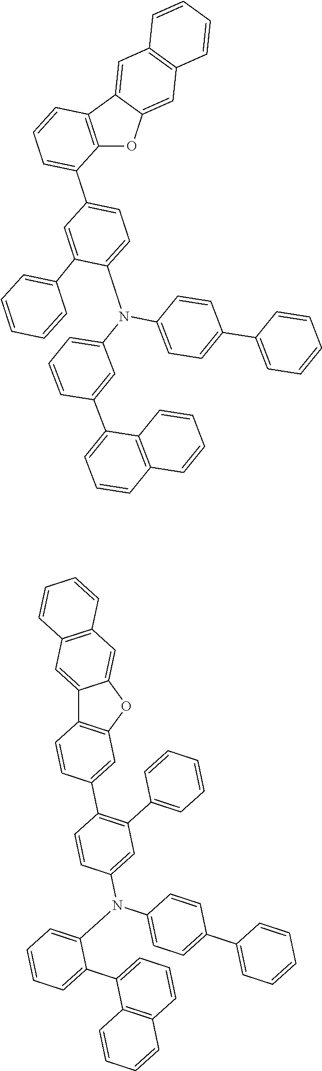 [SMILES: c1ccc(-c2ccc(N(c3cccc(-c4cccc5ccccc45)c3)c3ccc(-c4cccc5c4oc4cc6ccccc6cc45)cc3-c3ccccc3)cc2)cc1 ; c1ccc(-c2ccc(N(c3ccc(-c4ccc5c(c4)oc4cc6ccccc6cc45)c(-c4ccccc4)c3)c3ccccc3-c3cccc4ccccc34)cc2)cc1]

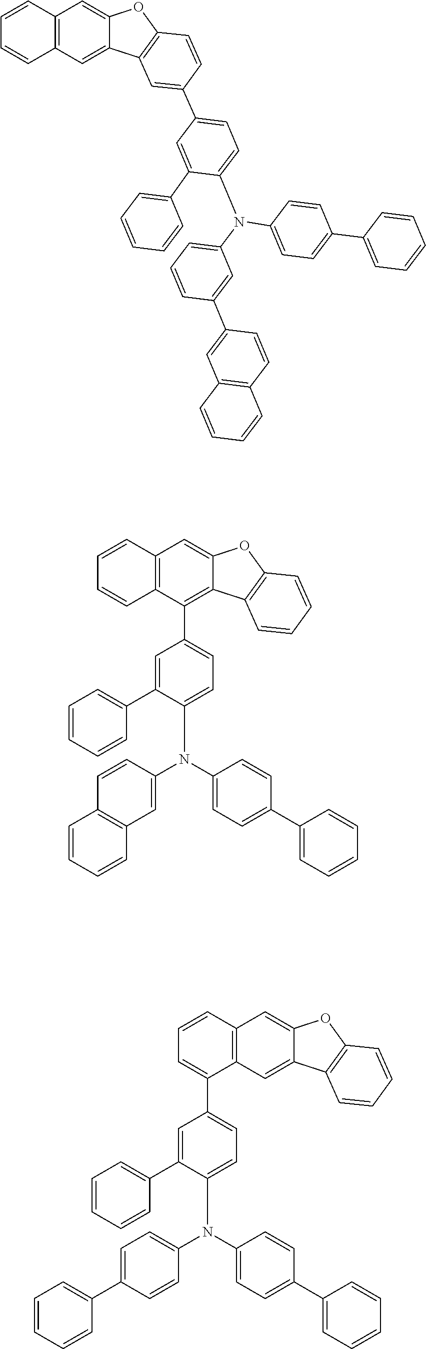 [SMILES: c1ccc(-c2ccc(N(c3cccc(-c4ccc5ccccc5c4)c3)c3ccc(-c4ccc5oc6cc7ccccc7cc6c5c4)cc3-c3ccccc3)cc2)cc1 ; c1ccc(-c2ccc(N(c3ccc4ccccc4c3)c3ccc(-c4c5ccccc5cc5oc6ccccc6c45)cc3-c3ccccc3)cc2)cc1 ; c1ccc(-c2ccc(N(c3ccc(-c4ccccc4)cc3)c3ccc(-c4cccc5cc6oc7ccccc7c6cc45)cc3-c3ccccc3)cc2)cc1]

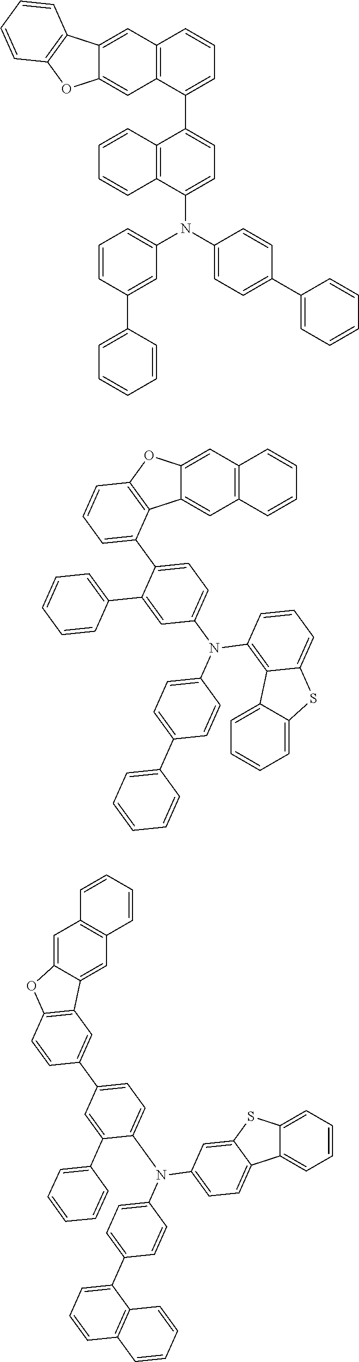 [SMILES: c1ccc(-c2ccc(N(c3cccc(-c4ccccc4)c3)c3ccc(-c4cccc5cc6c(cc45)oc4ccccc46)c4ccccc34)cc2)cc1 ; c1ccc(-c2ccc(N(c3ccc(-c4cccc5oc6cc7ccccc7cc6c45)c(-c4ccccc4)c3)c3cccc4sc5ccccc5c34)cc2)cc1 ; c1ccc(-c2cc(-c3ccc4oc5cc6ccccc6cc5c4c3)ccc2N(c2ccc(-c3cccc4ccccc34)cc2)c2ccc3c(c2)sc2ccccc23)cc1]

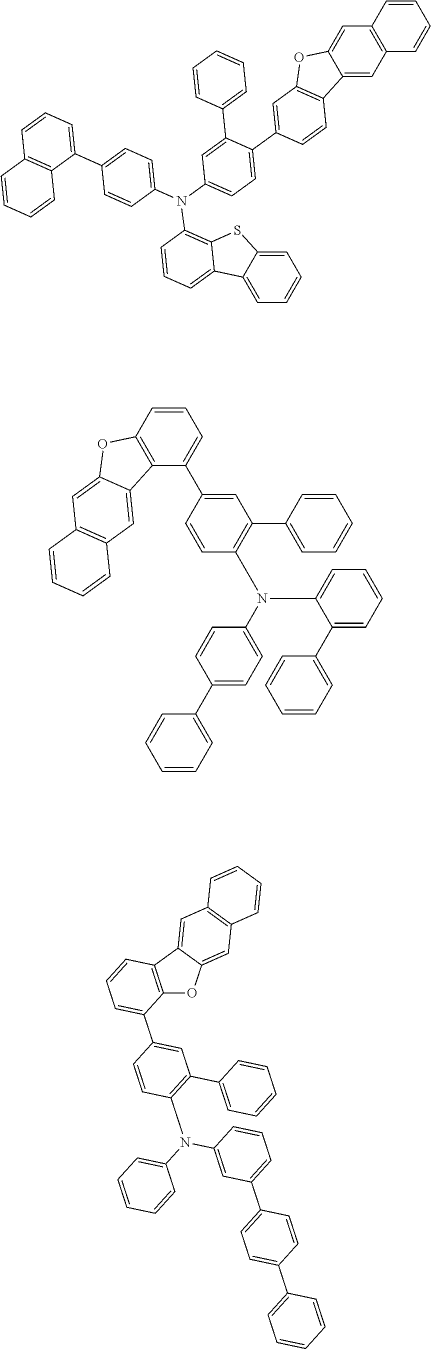 [SMILES: c1ccc(-c2cc(N(c3ccc(-c4cccc5ccccc45)cc3)c3cccc4c3sc3ccccc34)ccc2-c2ccc3c(c2)oc2cc4ccccc4cc23)cc1 ; c1ccc(-c2ccc(N(c3ccccc3-c3ccccc3)c3ccc(-c4cccc5oc6cc7ccccc7cc6c45)cc3-c3ccccc3)cc2)cc1 ; c1ccc(-c2ccc(-c3cccc(N(c4ccccc4)c4ccc(-c5cccc6c5oc5cc7ccccc7cc56)cc4-c4ccccc4)c3)cc2)cc1]

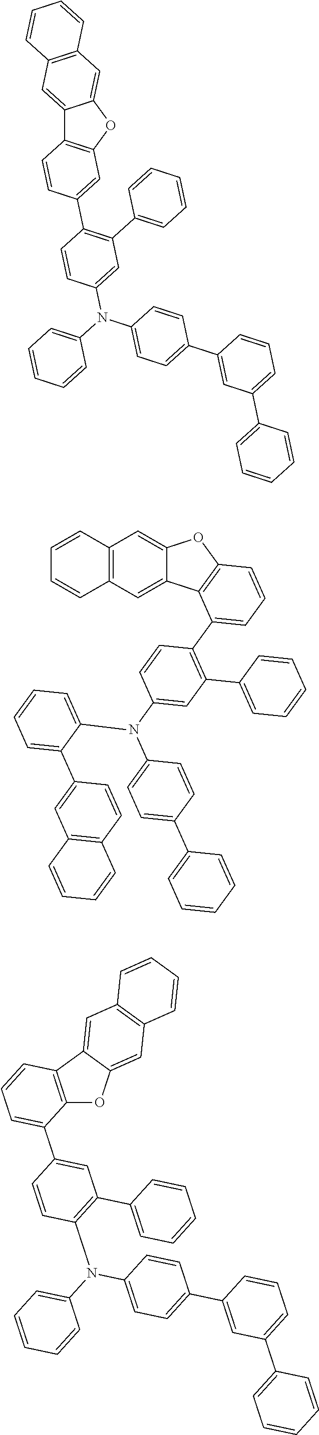 [SMILES: c1ccc(-c2cccc(-c3ccc(N(c4ccccc4)c4ccc(-c5ccc6c(c5)oc5cc7ccccc7cc56)c(-c5ccccc5)c4)cc3)c2)cc1 ; c1ccc(-c2ccc(N(c3ccc(-c4cccc5oc6cc7ccccc7cc6c45)c(-c4ccccc4)c3)c3ccccc3-c3ccc4ccccc4c3)cc2)cc1 ; c1ccc(-c2cccc(-c3ccc(N(c4ccccc4)c4ccc(-c5cccc6c5oc5cc7ccccc7cc56)cc4-c4ccccc4)cc3)c2)cc1]

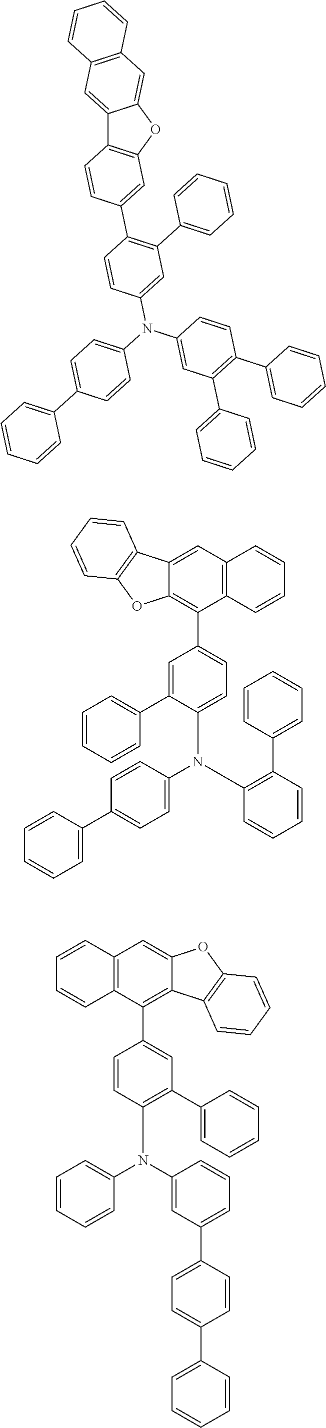 [SMILES: c1ccc(-c2ccc(N(c3ccc(-c4ccccc4)c(-c4ccccc4)c3)c3ccc(-c4ccc5c(c4)oc4cc6ccccc6cc45)c(-c4ccccc4)c3)cc2)cc1 ; c1ccc(-c2ccc(N(c3ccccc3-c3ccccc3)c3ccc(-c4c5ccccc5cc5c4oc4ccccc45)cc3-c3ccccc3)cc2)cc1 ; c1ccc(-c2ccc(-c3cccc(N(c4ccccc4)c4ccc(-c5c6ccccc6cc6oc7ccccc7c56)cc4-c4ccccc4)c3)cc2)cc1]

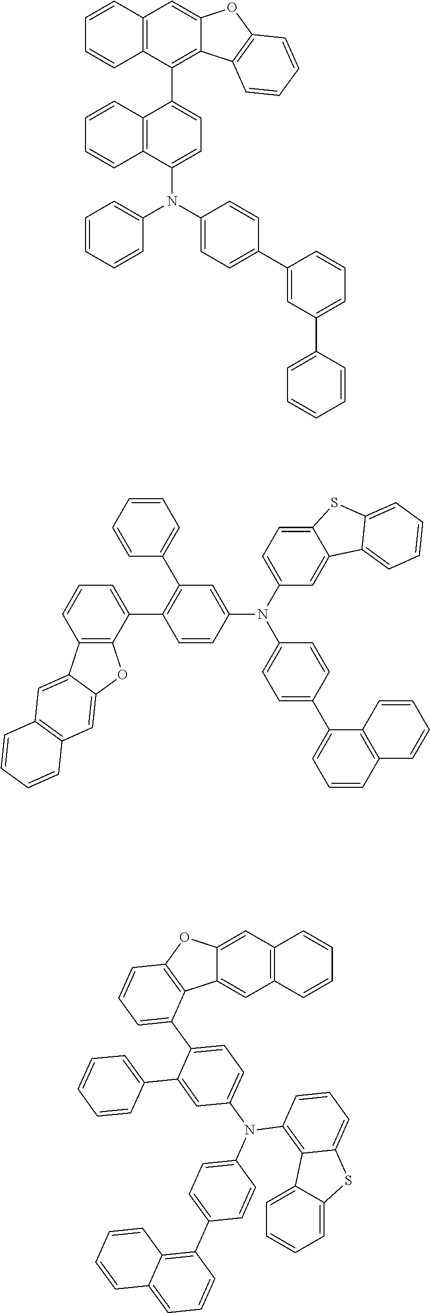 [SMILES: c1ccc(-c2cccc(-c3ccc(N(c4ccccc4)c4ccc(-c5c6ccccc6cc6oc7ccccc7c56)c5ccccc45)cc3)c2)cc1 ; c1ccc(-c2cc(N(c3ccc(-c4cccc5ccccc45)cc3)c3ccc4sc5ccccc5c4c3)ccc2-c2cccc3c2oc2cc4ccccc4cc23)cc1 ; c1ccc(-c2cc(N(c3ccc(-c4cccc5ccccc45)cc3)c3cccc4sc5ccccc5c34)ccc2-c2cccc3oc4cc5ccccc5cc4c23)cc1]

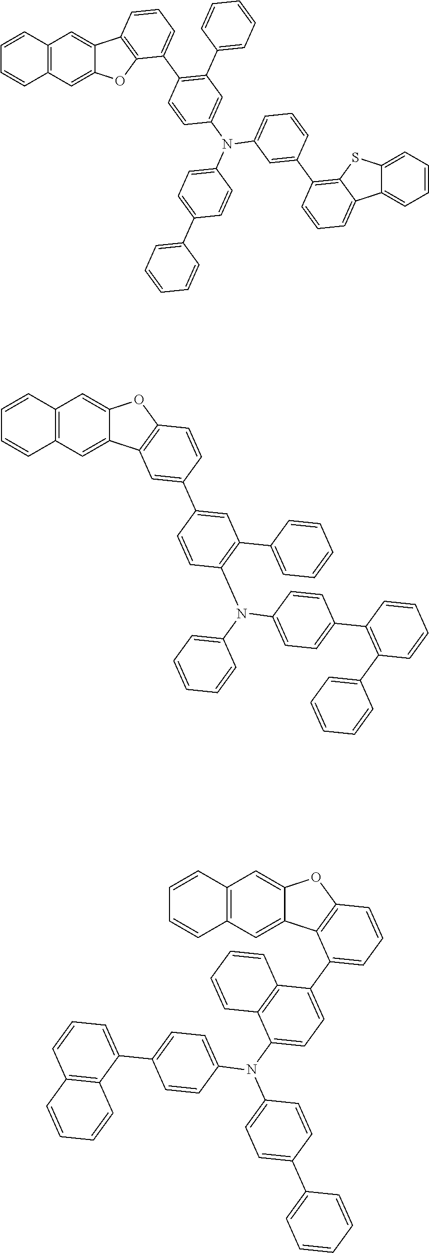 [SMILES: c1ccc(-c2ccc(N(c3cccc(-c4cccc5c4sc4ccccc45)c3)c3ccc(-c4cccc5c4oc4cc6ccccc6cc45)c(-c4ccccc4)c3)cc2)cc1 ; c1ccc(-c2ccccc2-c2ccc(N(c3ccccc3)c3ccc(-c4ccc5oc6cc7ccccc7cc6c5c4)cc3-c3ccccc3)cc2)cc1 ; c1ccc(-c2ccc(N(c3ccc(-c4cccc5ccccc45)cc3)c3ccc(-c4cccc5oc6cc7ccccc7cc6c45)c4ccccc34)cc2)cc1]

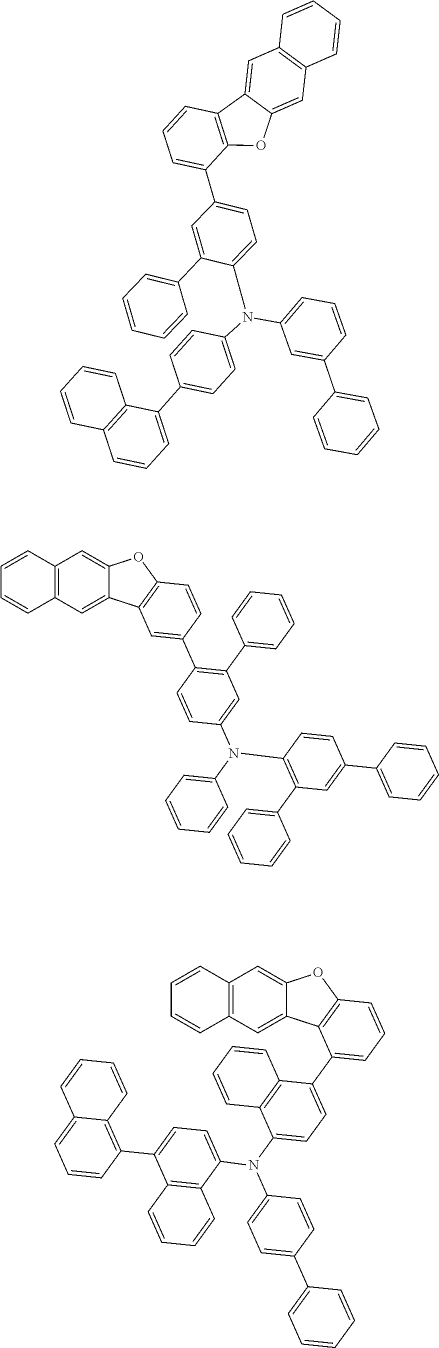 [SMILES: c1ccc(-c2cccc(N(c3ccc(-c4cccc5ccccc45)cc3)c3ccc(-c4cccc5c4oc4cc6ccccc6cc45)cc3-c3ccccc3)c2)cc1 ; c1ccc(-c2ccc(N(c3ccccc3)c3ccc(-c4ccc5oc6cc7ccccc7cc6c5c4)c(-c4ccccc4)c3)c(-c3ccccc3)c2)cc1 ; c1ccc(-c2ccc(N(c3ccc(-c4cccc5ccccc45)c4ccccc34)c3ccc(-c4cccc5oc6cc7ccccc7cc6c45)c4ccccc34)cc2)cc1]

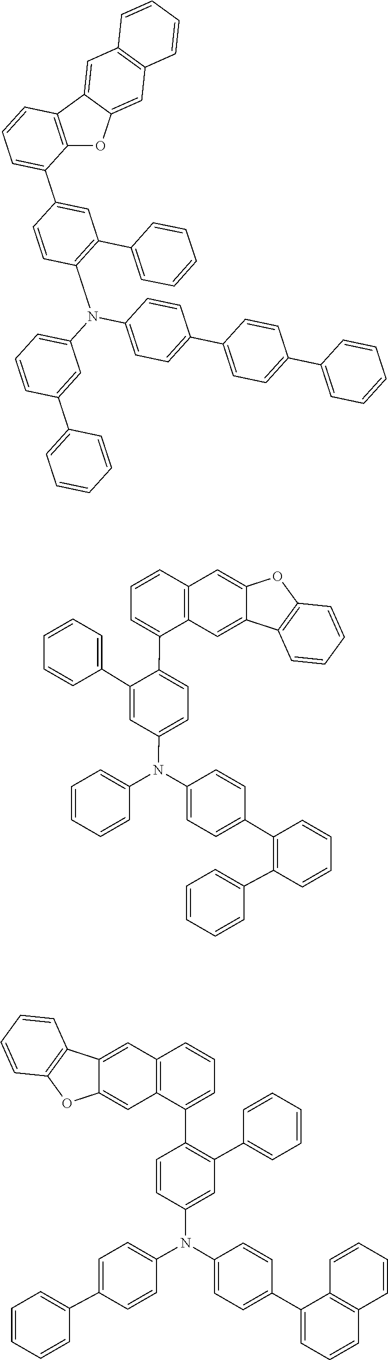 [SMILES: c1ccc(-c2ccc(-c3ccc(N(c4cccc(-c5ccccc5)c4)c4ccc(-c5cccc6c5oc5cc7ccccc7cc56)cc4-c4ccccc4)cc3)cc2)cc1 ; c1ccc(-c2ccccc2-c2ccc(N(c3ccccc3)c3ccc(-c4cccc5cc6oc7ccccc7c6cc45)c(-c4ccccc4)c3)cc2)cc1 ; c1ccc(-c2ccc(N(c3ccc(-c4cccc5ccccc45)cc3)c3ccc(-c4cccc5cc6c(cc45)oc4ccccc46)c(-c4ccccc4)c3)cc2)cc1]

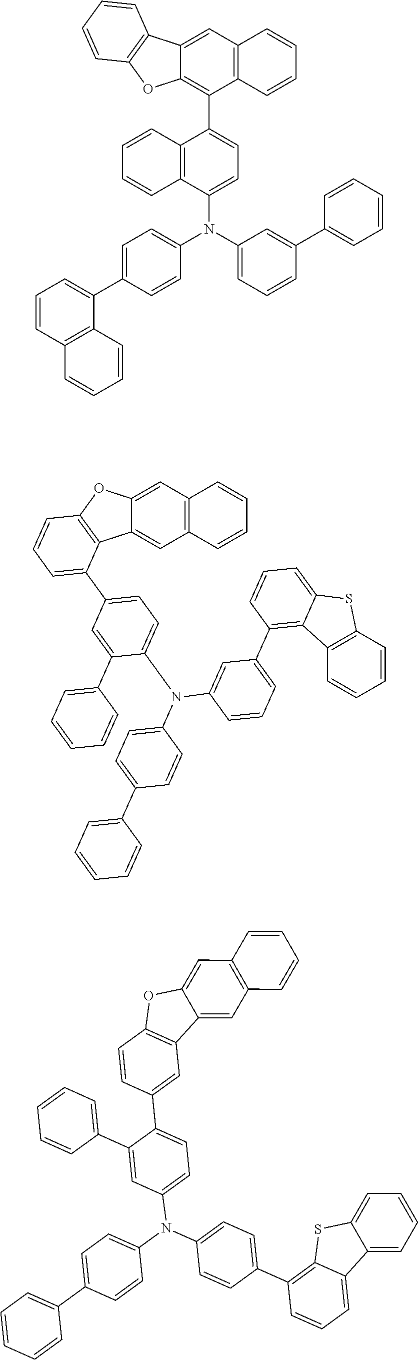 [SMILES: c1ccc(-c2cccc(N(c3ccc(-c4cccc5ccccc45)cc3)c3ccc(-c4c5ccccc5cc5c4oc4ccccc45)c4ccccc34)c2)cc1 ; c1ccc(-c2ccc(N(c3cccc(-c4cccc5sc6ccccc6c45)c3)c3ccc(-c4cccc5oc6cc7ccccc7cc6c45)cc3-c3ccccc3)cc2)cc1 ; c1ccc(-c2ccc(N(c3ccc(-c4cccc5c4sc4ccccc45)cc3)c3ccc(-c4ccc5oc6cc7ccccc7cc6c5c4)c(-c4ccccc4)c3)cc2)cc1]

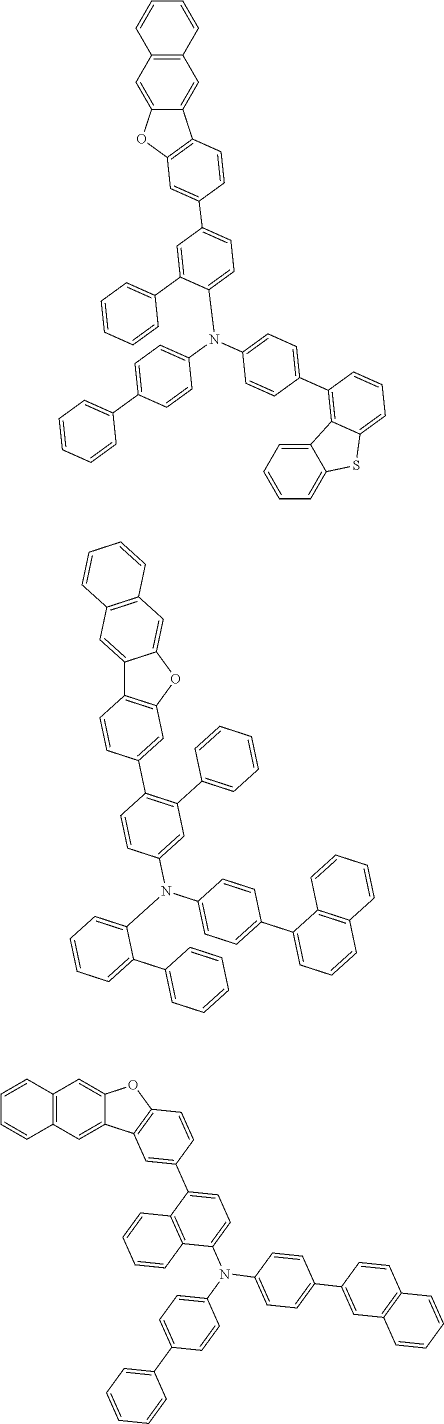 [SMILES: c1ccc(-c2ccc(N(c3ccc(-c4cccc5sc6ccccc6c45)cc3)c3ccc(-c4ccc5c(c4)oc4cc6ccccc6cc45)cc3-c3ccccc3)cc2)cc1 ; c1ccc(-c2cc(N(c3ccc(-c4cccc5ccccc45)cc3)c3ccccc3-c3ccccc3)ccc2-c2ccc3c(c2)oc2cc4ccccc4cc23)cc1 ; c1ccc(-c2ccc(N(c3ccc(-c4ccc5ccccc5c4)cc3)c3ccc(-c4ccc5oc6cc7ccccc7cc6c5c4)c4ccccc34)cc2)cc1]

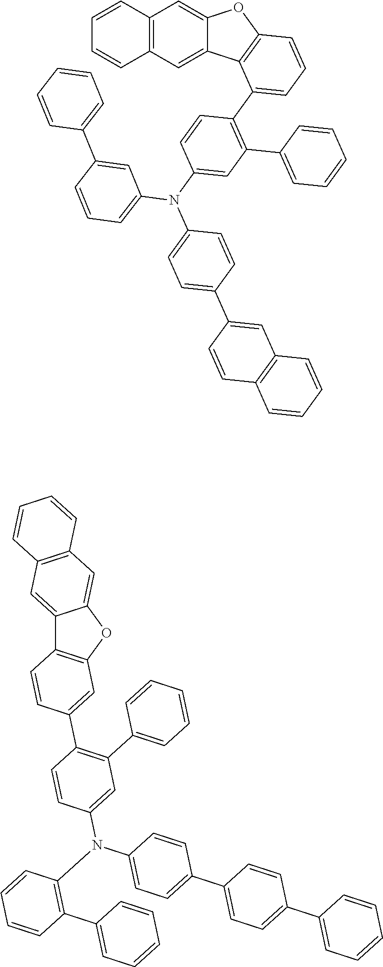 [SMILES: c1ccc(-c2cccc(N(c3ccc(-c4ccc5ccccc5c4)cc3)c3ccc(-c4cccc5oc6cc7ccccc7cc6c45)c(-c4ccccc4)c3)c2)cc1 ; c1ccc(-c2ccc(-c3ccc(N(c4ccc(-c5ccc6c(c5)oc5cc7ccccc7cc56)c(-c5ccccc5)c4)c4ccccc4-c4ccccc4)cc3)cc2)cc1]

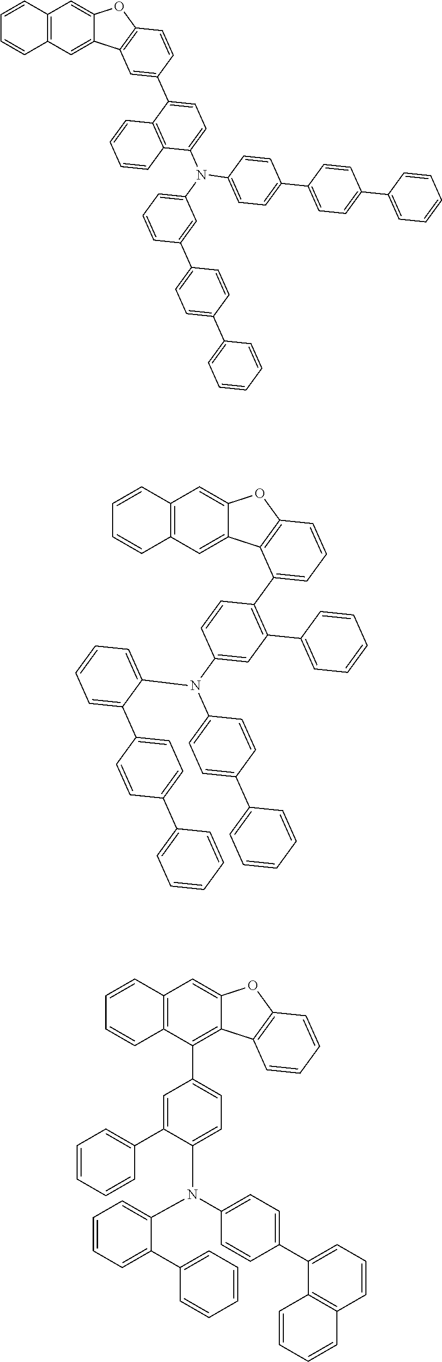 [SMILES: c1ccc(-c2ccc(-c3ccc(N(c4cccc(-c5ccc(-c6ccccc6)cc5)c4)c4ccc(-c5ccc6oc7cc8ccccc8cc7c6c5)c5ccccc45)cc3)cc2)cc1 ; c1ccc(-c2ccc(-c3ccccc3N(c3ccc(-c4ccccc4)cc3)c3ccc(-c4cccc5oc6cc7ccccc7cc6c45)c(-c4ccccc4)c3)cc2)cc1 ; c1ccc(-c2ccccc2N(c2ccc(-c3cccc4ccccc34)cc2)c2ccc(-c3c4ccccc4cc4oc5ccccc5c34)cc2-c2ccccc2)cc1]

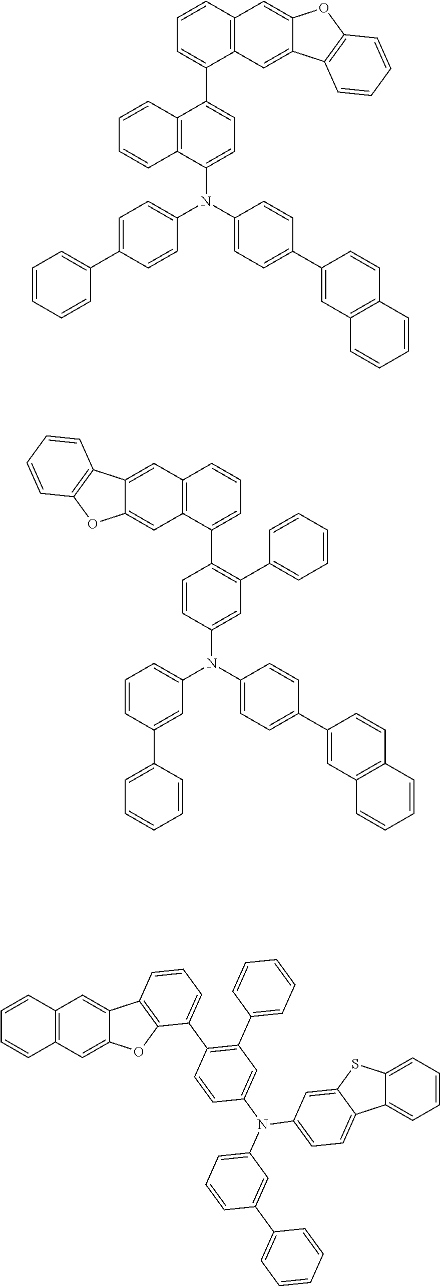 [SMILES: c1ccc(-c2ccc(N(c3ccc(-c4ccc5ccccc5c4)cc3)c3ccc(-c4cccc5cc6oc7ccccc7c6cc45)c4ccccc34)cc2)cc1 ; c1ccc(-c2cccc(N(c3ccc(-c4ccc5ccccc5c4)cc3)c3ccc(-c4cccc5cc6c(cc45)oc4ccccc46)c(-c4ccccc4)c3)c2)cc1 ; c1ccc(-c2cccc(N(c3ccc(-c4cccc5c4oc4cc6ccccc6cc45)c(-c4ccccc4)c3)c3ccc4c(c3)sc3ccccc34)c2)cc1]

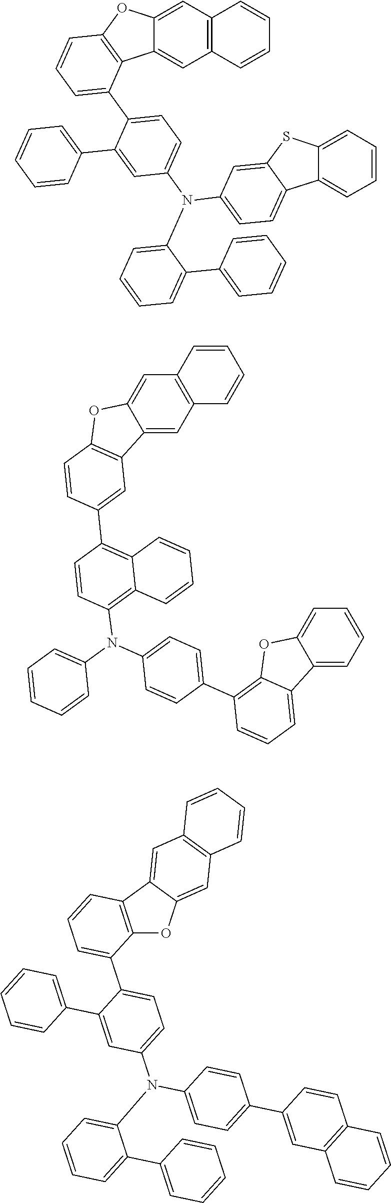 [SMILES: c1ccc(-c2cc(N(c3ccc4c(c3)sc3ccccc34)c3ccccc3-c3ccccc3)ccc2-c2cccc3oc4cc5ccccc5cc4c23)cc1 ; c1ccc(N(c2ccc(-c3cccc4c3oc3ccccc34)cc2)c2ccc(-c3ccc4oc5cc6ccccc6cc5c4c3)c3ccccc23)cc1 ; c1ccc(-c2cc(N(c3ccc(-c4ccc5ccccc5c4)cc3)c3ccccc3-c3ccccc3)ccc2-c2cccc3c2oc2cc4ccccc4cc23)cc1]

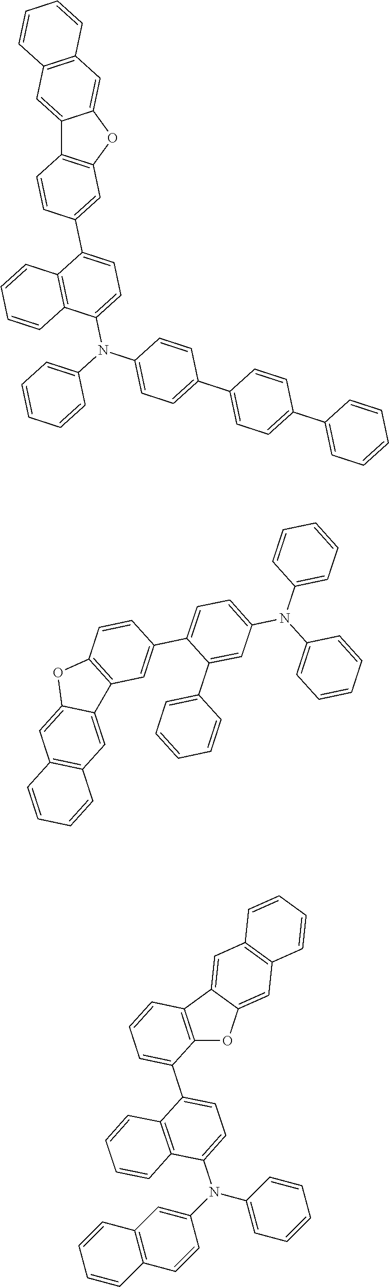 [SMILES: c1ccc(-c2ccc(-c3ccc(N(c4ccccc4)c4ccc(-c5ccc6c(c5)oc5cc7ccccc7cc56)c5ccccc45)cc3)cc2)cc1 ; c1ccc(-c2cc(N(c3ccccc3)c3ccccc3)ccc2-c2ccc3oc4cc5ccccc5cc4c3c2)cc1 ; c1ccc(N(c2ccc3ccccc3c2)c2ccc(-c3cccc4c3oc3cc5ccccc5cc34)c3ccccc23)cc1]

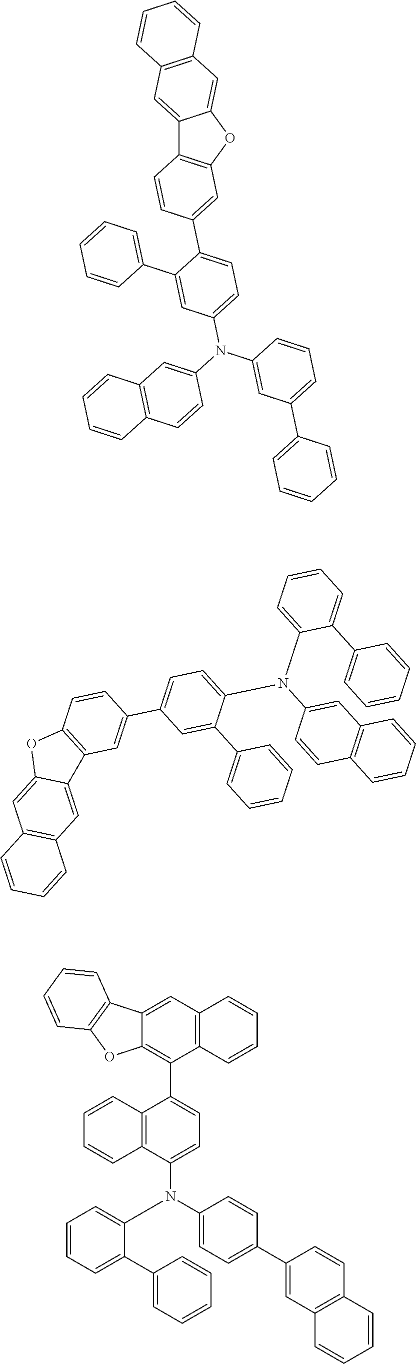 [SMILES: c1ccc(-c2cccc(N(c3ccc(-c4ccc5c(c4)oc4cc6ccccc6cc45)c(-c4ccccc4)c3)c3ccc4ccccc4c3)c2)cc1 ; c1ccc(-c2ccccc2N(c2ccc3ccccc3c2)c2ccc(-c3ccc4oc5cc6ccccc6cc5c4c3)cc2-c2ccccc2)cc1 ; c1ccc(-c2ccccc2N(c2ccc(-c3ccc4ccccc4c3)cc2)c2ccc(-c3c4ccccc4cc4c3oc3ccccc34)c3ccccc23)cc1]

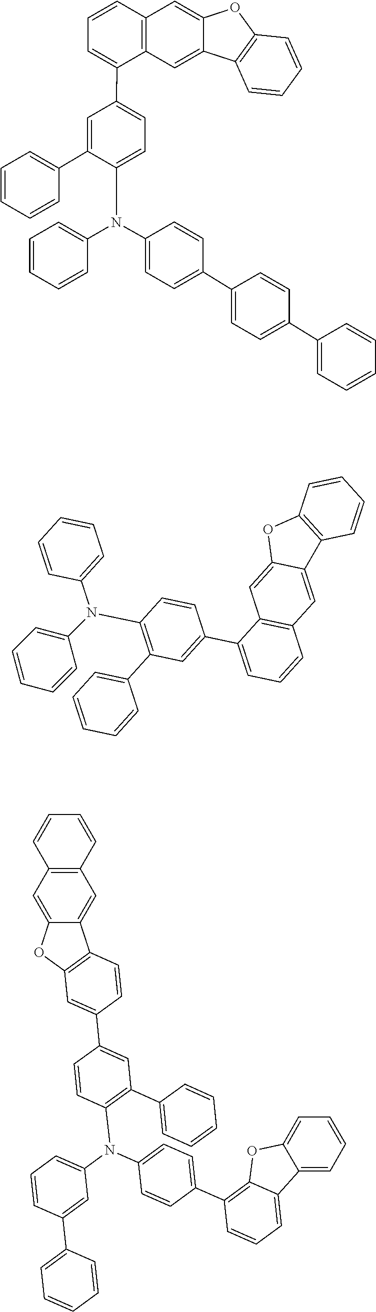 [SMILES: c1ccc(-c2ccc(-c3ccc(N(c4ccccc4)c4ccc(-c5cccc6cc7oc8ccccc8c7cc56)cc4-c4ccccc4)cc3)cc2)cc1 ; c1ccc(-c2cc(-c3cccc4cc5c(cc34)oc3ccccc35)ccc2N(c2ccccc2)c2ccccc2)cc1 ; c1ccc(-c2cccc(N(c3ccc(-c4cccc5c4oc4ccccc45)cc3)c3ccc(-c4ccc5c(c4)oc4cc6ccccc6cc45)cc3-c3ccccc3)c2)cc1]

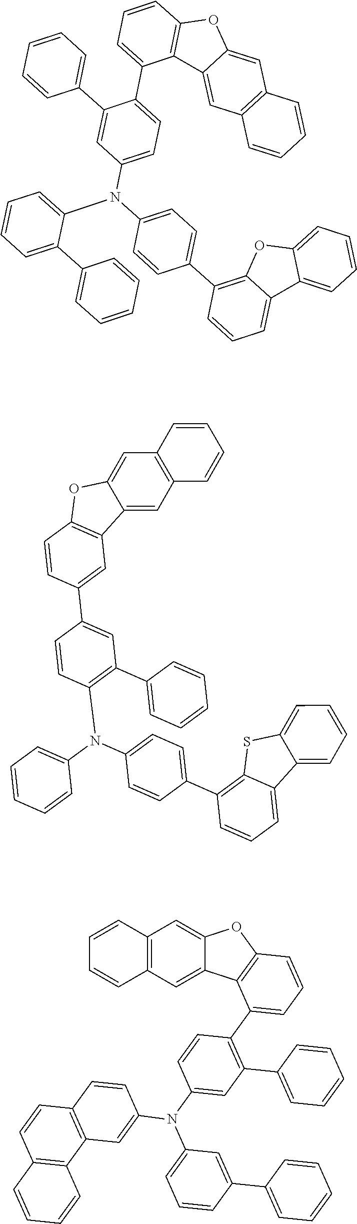 [SMILES: c1ccc(-c2cc(N(c3ccc(-c4cccc5c4oc4ccccc45)cc3)c3ccccc3-c3ccccc3)ccc2-c2cccc3oc4cc5ccccc5cc4c23)cc1 ; c1ccc(-c2cc(-c3ccc4oc5cc6ccccc6cc5c4c3)ccc2N(c2ccccc2)c2ccc(-c3cccc4c3sc3ccccc34)cc2)cc1 ; c1ccc(-c2cccc(N(c3ccc(-c4cccc5oc6cc7ccccc7cc6c45)c(-c4ccccc4)c3)c3ccc4ccc5ccccc5c4c3)c2)cc1]

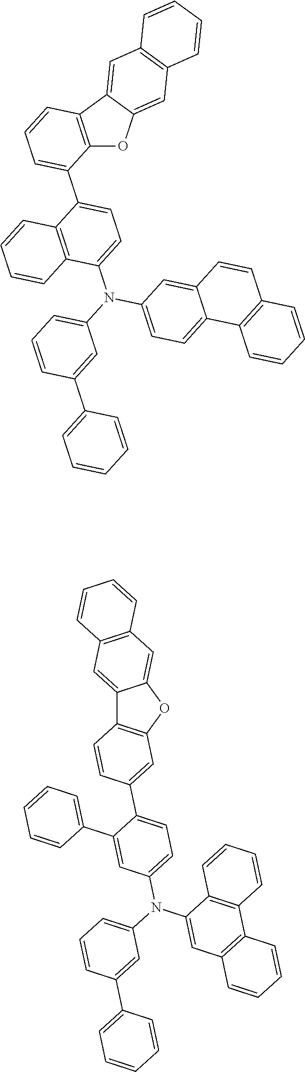 [SMILES: c1ccc(-c2cccc(N(c3ccc4c(ccc5ccccc54)c3)c3ccc(-c4cccc5c4oc4cc6ccccc6cc45)c4ccccc34)c2)cc1 ; c1ccc(-c2cccc(N(c3ccc(-c4ccc5c(c4)oc4cc6ccccc6cc45)c(-c4ccccc4)c3)c3cc4ccccc4c4ccccc34)c2)cc1]

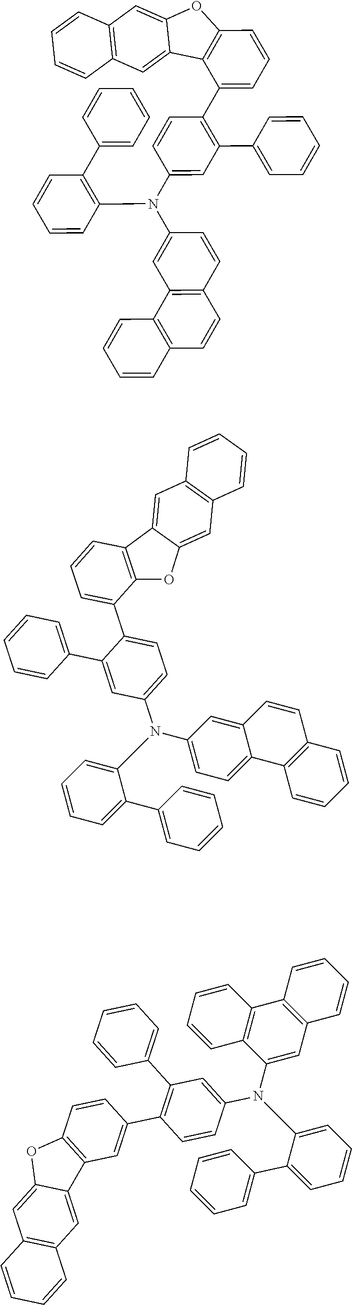 [SMILES: c1ccc(-c2cc(N(c3ccc4ccc5ccccc5c4c3)c3ccccc3-c3ccccc3)ccc2-c2cccc3oc4cc5ccccc5cc4c23)cc1 ; c1ccc(-c2cc(N(c3ccc4c(ccc5ccccc54)c3)c3ccccc3-c3ccccc3)ccc2-c2cccc3c2oc2cc4ccccc4cc23)cc1 ; c1ccc(-c2cc(N(c3ccccc3-c3ccccc3)c3cc4ccccc4c4ccccc34)ccc2-c2ccc3oc4cc5ccccc5cc4c3c2)cc1]

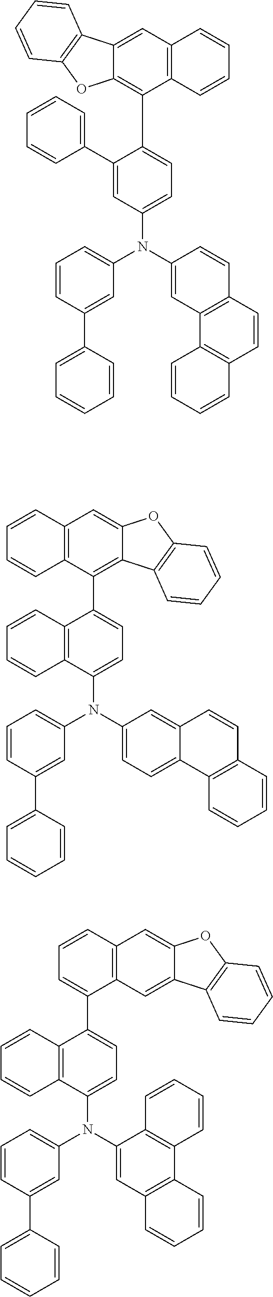 [SMILES: c1ccc(-c2cccc(N(c3ccc(-c4c5ccccc5cc5c4oc4ccccc45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4c5ccccc5cc5oc6ccccc6c45)c4ccccc34)c2)cc1 ; c1ccc(-c2cccc(N(c3ccc(-c4cccc5cc6oc7ccccc7c6cc45)c4ccccc34)c3cc4ccccc4c4ccccc34)c2)cc1]

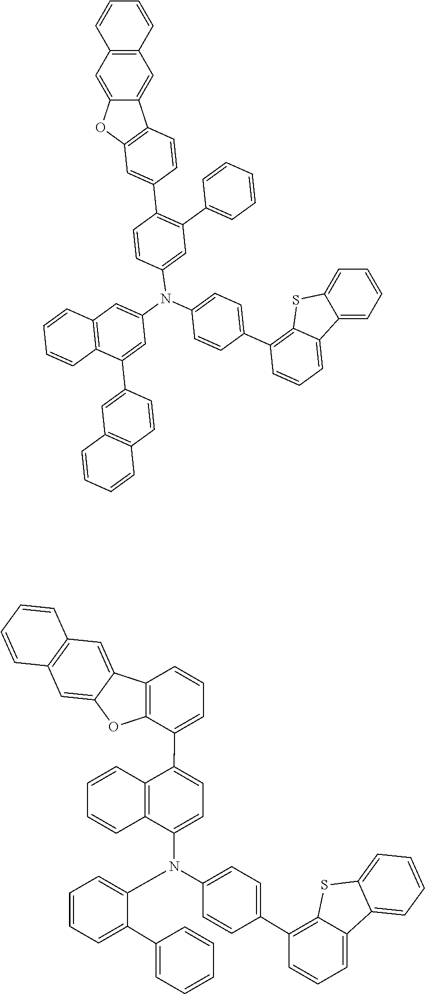 [SMILES: c1ccc(-c2cc(N(c3ccc(-c4cccc5c4sc4ccccc45)cc3)c3cc(-c4ccc5ccccc5c4)c4ccccc4c3)ccc2-c2ccc3c(c2)oc2cc4ccccc4cc23)cc1 ; c1ccc(-c2ccccc2N(c2ccc(-c3cccc4c3sc3ccccc34)cc2)c2ccc(-c3cccc4c3oc3cc5ccccc5cc34)c3ccccc23)cc1]

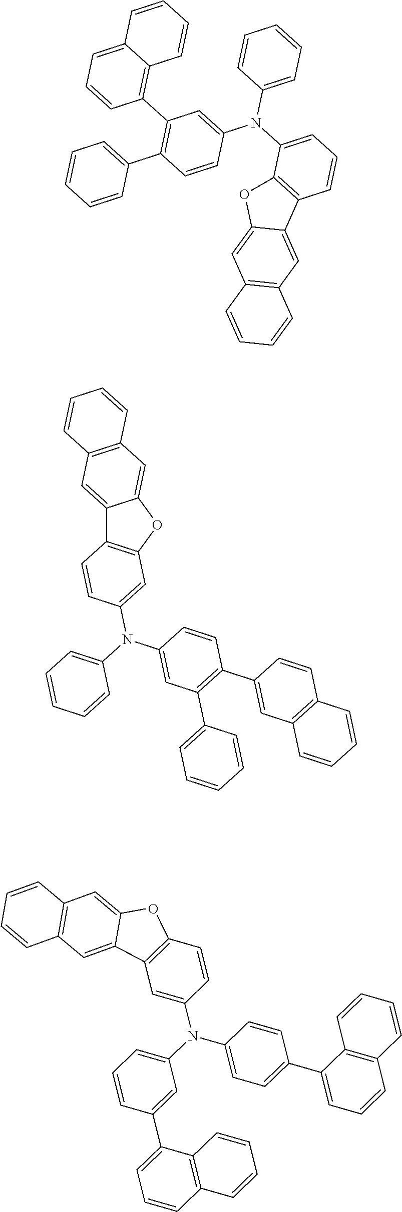 [SMILES: c1ccc(-c2ccc(N(c3ccccc3)c3cccc4c3oc3cc5ccccc5cc34)cc2-c2cccc3ccccc23)cc1 ; c1ccc(-c2cc(N(c3ccccc3)c3ccc4c(c3)oc3cc5ccccc5cc34)ccc2-c2ccc3ccccc3c2)cc1 ; c1cc(-c2cccc3ccccc23)cc(N(c2ccc(-c3cccc4ccccc34)cc2)c2ccc3oc4cc5ccccc5cc4c3c2)c1]

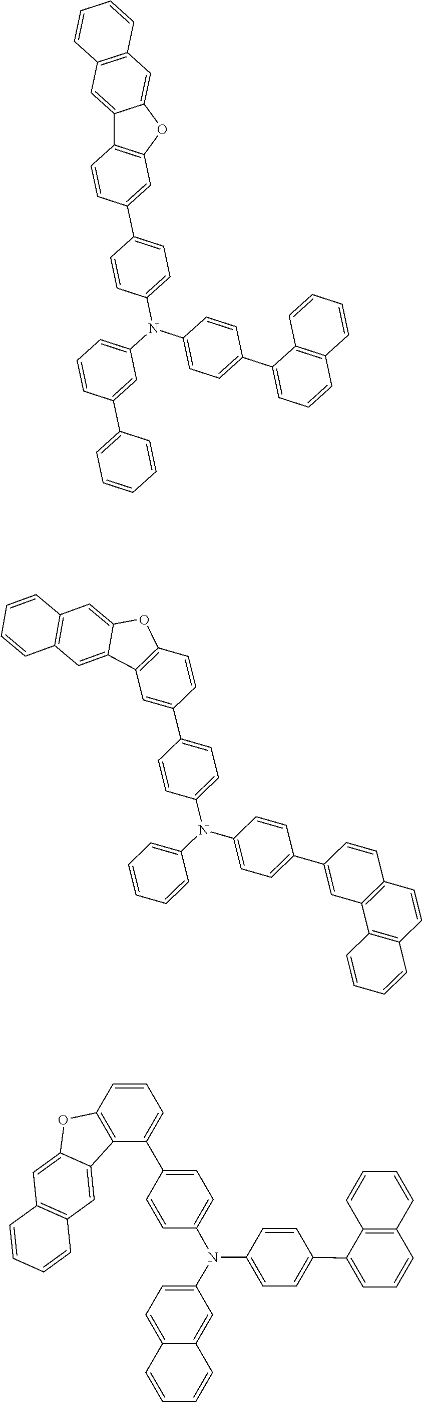 [SMILES: c1ccc(-c2cccc(N(c3ccc(-c4ccc5c(c4)oc4cc6ccccc6cc45)cc3)c3ccc(-c4cccc5ccccc45)cc3)c2)cc1 ; c1ccc(N(c2ccc(-c3ccc4ccc5ccccc5c4c3)cc2)c2ccc(-c3ccc4oc5cc6ccccc6cc5c4c3)cc2)cc1 ; c1ccc2cc(N(c3ccc(-c4cccc5ccccc45)cc3)c3ccc(-c4cccc5oc6cc7ccccc7cc6c45)cc3)ccc2c1]

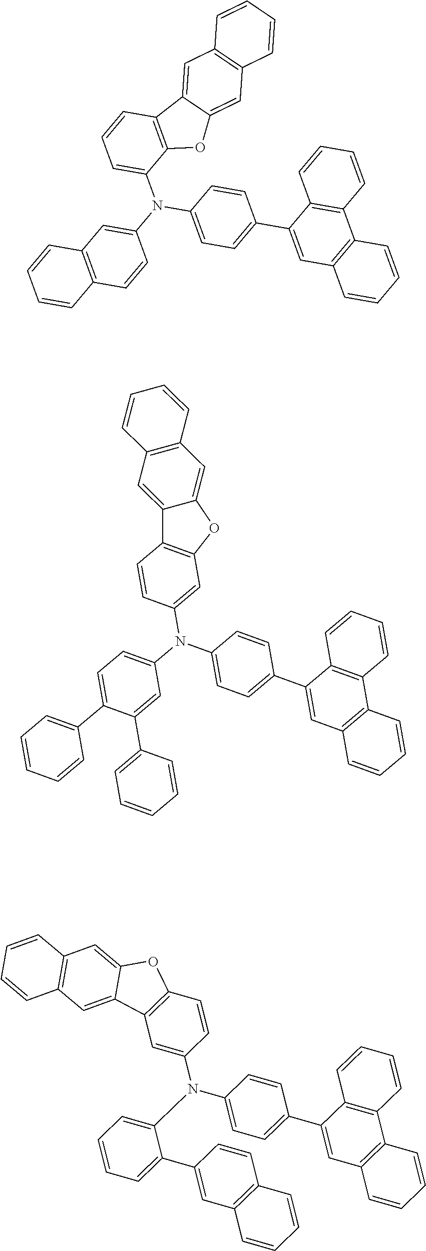 [SMILES: c1ccc2cc(N(c3ccc(-c4cc5ccccc5c5ccccc45)cc3)c3cccc4c3oc3cc5ccccc5cc34)ccc2c1 ; c1ccc(-c2ccc(N(c3ccc(-c4cc5ccccc5c5ccccc45)cc3)c3ccc4c(c3)oc3cc5ccccc5cc34)cc2-c2ccccc2)cc1 ; c1ccc(N(c2ccc(-c3cc4ccccc4c4ccccc34)cc2)c2ccc3oc4cc5ccccc5cc4c3c2)c(-c2ccc3ccccc3c2)c1]

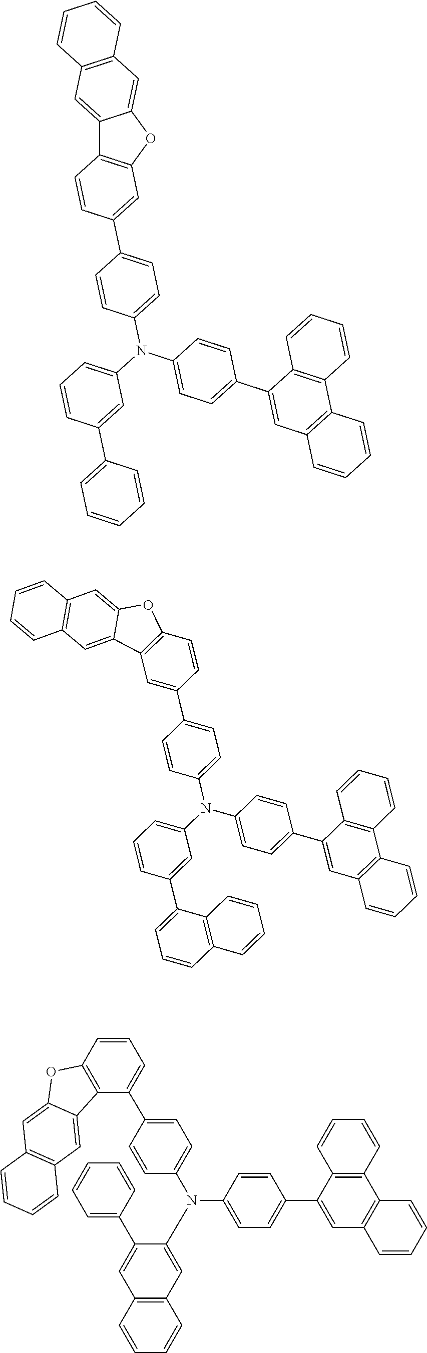 [SMILES: c1ccc(-c2cccc(N(c3ccc(-c4ccc5c(c4)oc4cc6ccccc6cc45)cc3)c3ccc(-c4cc5ccccc5c5ccccc45)cc3)c2)cc1 ; c1cc(-c2cccc3ccccc23)cc(N(c2ccc(-c3ccc4oc5cc6ccccc6cc5c4c3)cc2)c2ccc(-c3cc4ccccc4c4ccccc34)cc2)c1 ; c1ccc(-c2cc3ccccc3cc2N(c2ccc(-c3cc4ccccc4c4ccccc34)cc2)c2ccc(-c3cccc4oc5cc6ccccc6cc5c34)cc2)cc1]

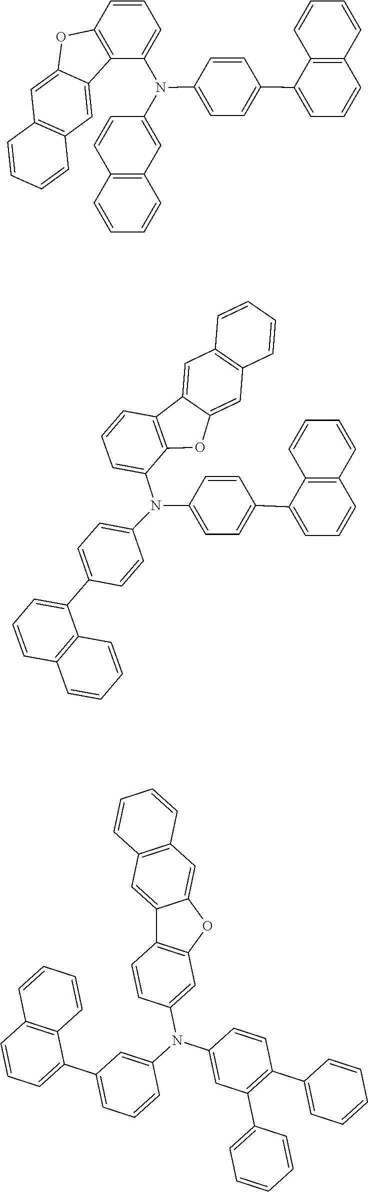 [SMILES: c1ccc2cc(N(c3ccc(-c4cccc5ccccc45)cc3)c3cccc4oc5cc6ccccc6cc5c34)ccc2c1 ; c1ccc2cc3c(cc2c1)oc1c(N(c2ccc(-c4cccc5ccccc45)cc2)c2ccc(-c4cccc5ccccc45)cc2)cccc13 ; c1ccc(-c2ccc(N(c3cccc(-c4cccc5ccccc45)c3)c3ccc4c(c3)oc3cc5ccccc5cc34)cc2-c2ccccc2)cc1]

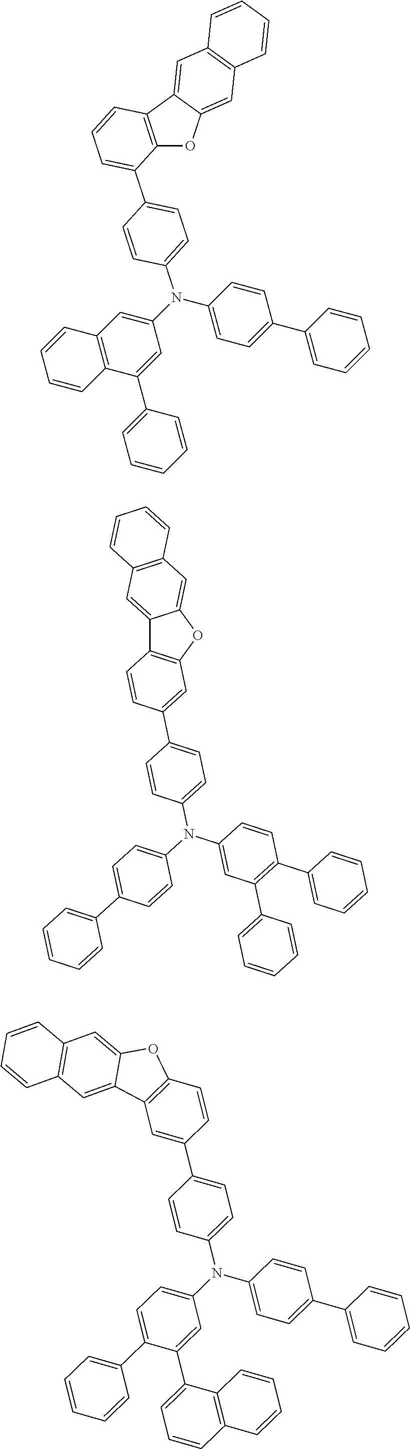

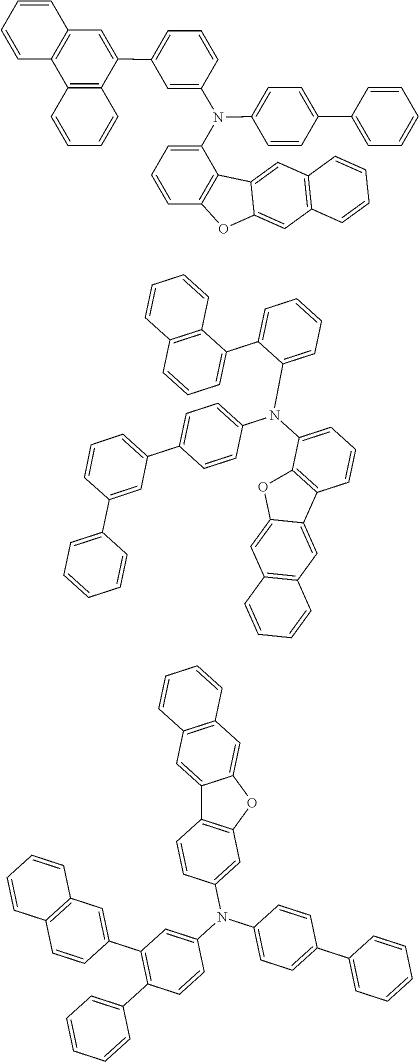 [SMILES: c1ccc(-c2ccc(N(c3cccc(-c4cc5ccccc5c5ccccc45)c3)c3cccc4oc5cc6ccccc6cc5c34)cc2)cc1 ; c1ccc(-c2cccc(-c3ccc(N(c4ccccc4-c4cccc5ccccc45)c4cccc5c4oc4cc6ccccc6cc45)cc3)c2)cc1 ; c1ccc(-c2ccc(N(c3ccc(-c4ccccc4)c(-c4ccc5ccccc5c4)c3)c3ccc4c(c3)oc3cc5ccccc5cc34)cc2)cc1]

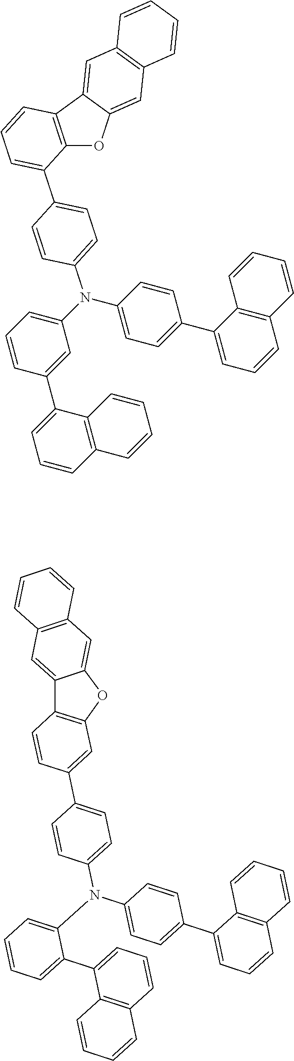 [SMILES: c1cc(-c2cccc3ccccc23)cc(N(c2ccc(-c3cccc4ccccc34)cc2)c2ccc(-c3cccc4c3oc3cc5ccccc5cc34)cc2)c1 ; c1ccc(N(c2ccc(-c3ccc4c(c3)oc3cc5ccccc5cc34)cc2)c2ccc(-c3cccc4ccccc34)cc2)c(-c2cccc3ccccc23)c1]

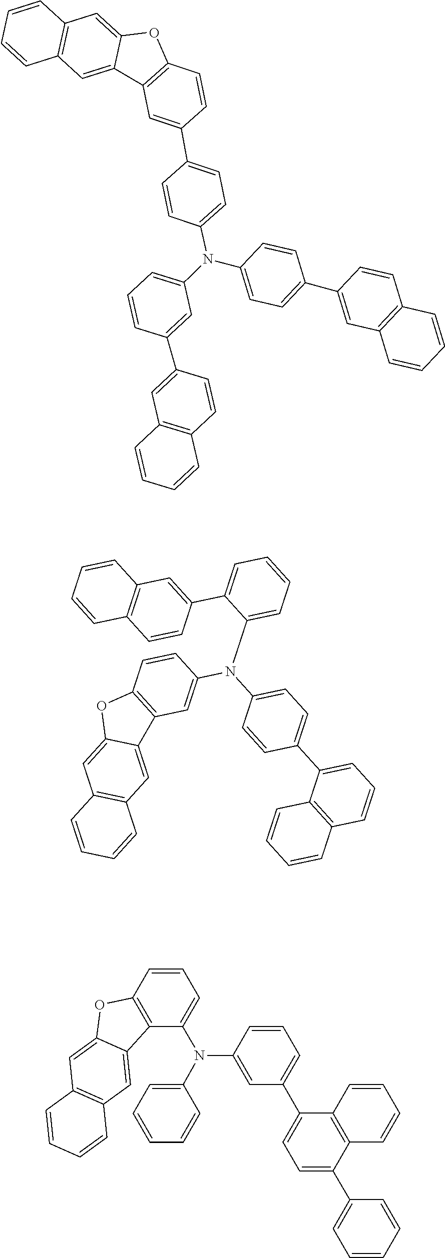 [SMILES: c1cc(-c2ccc3ccccc3c2)cc(N(c2ccc(-c3ccc4ccccc4c3)cc2)c2ccc(-c3ccc4oc5cc6ccccc6cc5c4c3)cc2)c1 ; c1ccc(N(c2ccc(-c3cccc4ccccc34)cc2)c2ccc3oc4cc5ccccc5cc4c3c2)c(-c2ccc3ccccc3c2)c1 ; c1ccc(-c2ccc(-c3cccc(N(c4ccccc4)c4cccc5oc6cc7ccccc7cc6c45)c3)c3ccccc23)cc1]

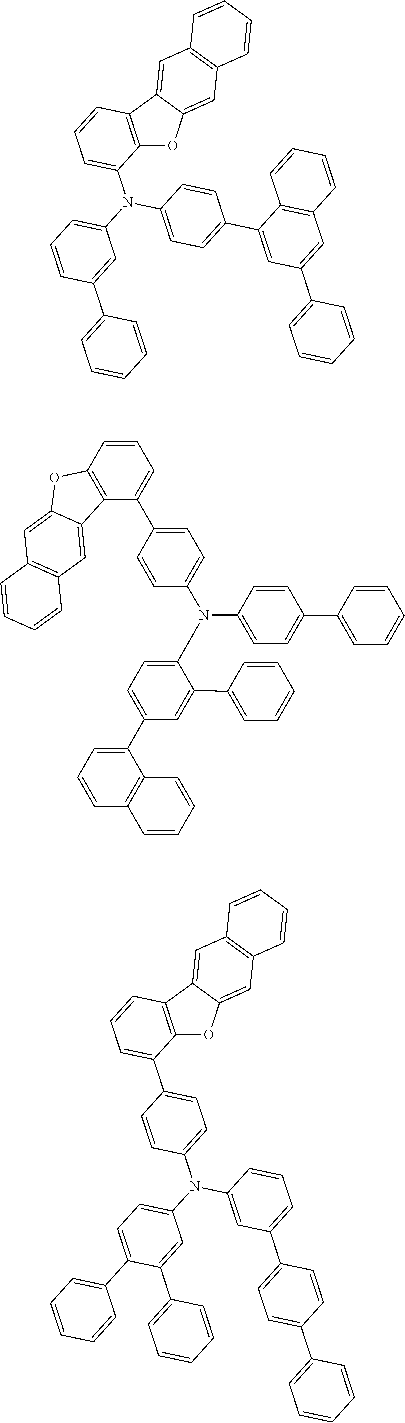 [SMILES: c1ccc(-c2cccc(N(c3ccc(-c4cc(-c5ccccc5)cc5ccccc45)cc3)c3cccc4c3oc3cc5ccccc5cc34)c2)cc1 ; c1ccc(-c2ccc(N(c3ccc(-c4cccc5oc6cc7ccccc7cc6c45)cc3)c3ccc(-c4cccc5ccccc45)cc3-c3ccccc3)cc2)cc1 ; c1ccc(-c2ccc(-c3cccc(N(c4ccc(-c5cccc6c5oc5cc7ccccc7cc56)cc4)c4ccc(-c5ccccc5)c(-c5ccccc5)c4)c3)cc2)cc1]

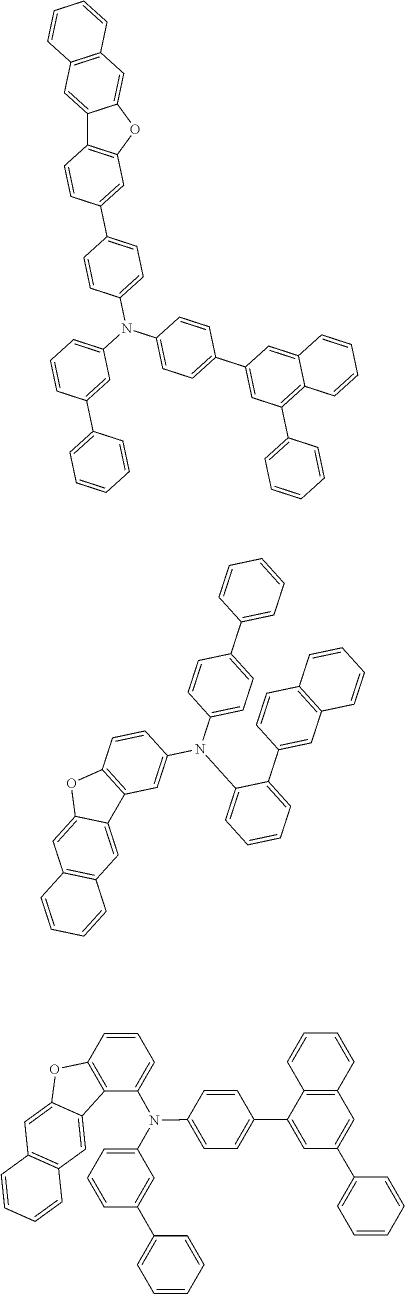 [SMILES: c1ccc(-c2cccc(N(c3ccc(-c4cc(-c5ccccc5)c5ccccc5c4)cc3)c3ccc(-c4ccc5c(c4)oc4cc6ccccc6cc45)cc3)c2)cc1 ; c1ccc(-c2ccc(N(c3ccc4oc5cc6ccccc6cc5c4c3)c3ccccc3-c3ccc4ccccc4c3)cc2)cc1 ; c1ccc(-c2cccc(N(c3ccc(-c4cc(-c5ccccc5)cc5ccccc45)cc3)c3cccc4oc5cc6ccccc6cc5c34)c2)cc1]

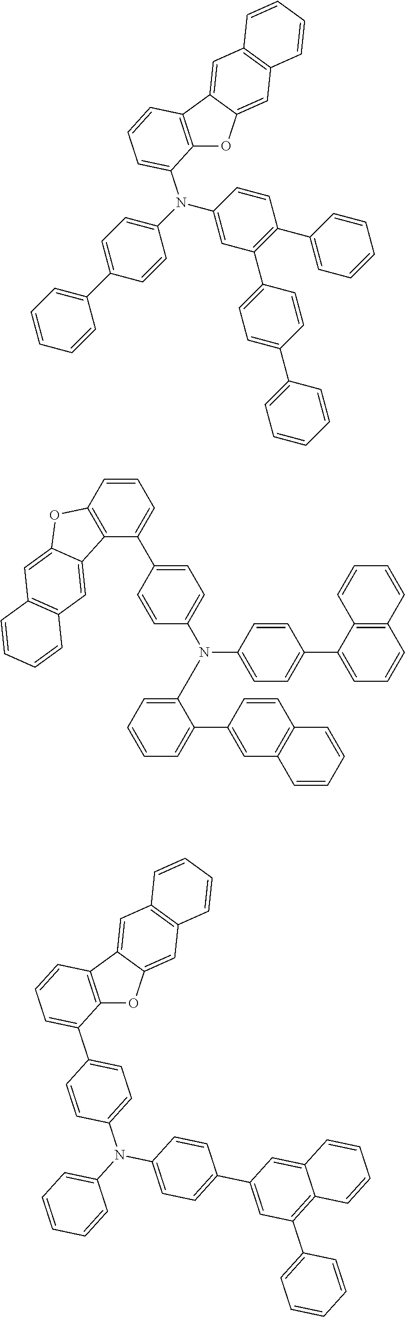 [SMILES: c1ccc(-c2ccc(-c3cc(N(c4ccc(-c5ccccc5)cc4)c4cccc5c4oc4cc6ccccc6cc45)ccc3-c3ccccc3)cc2)cc1 ; c1ccc(N(c2ccc(-c3cccc4ccccc34)cc2)c2ccc(-c3cccc4oc5cc6ccccc6cc5c34)cc2)c(-c2ccc3ccccc3c2)c1 ; c1ccc(-c2cc(-c3ccc(N(c4ccccc4)c4ccc(-c5cccc6c5oc5cc7ccccc7cc56)cc4)cc3)cc3ccccc23)cc1]

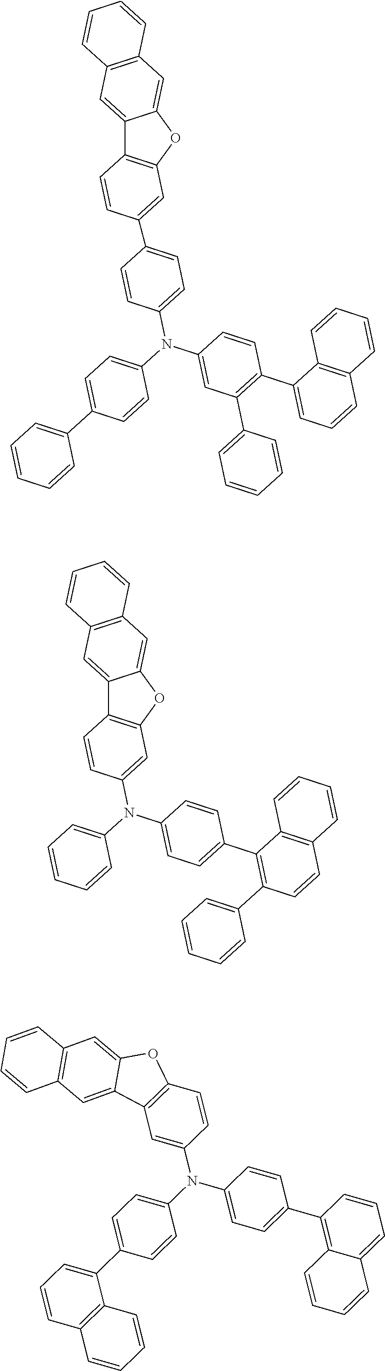 [SMILES: c1ccc(-c2ccc(N(c3ccc(-c4ccc5c(c4)oc4cc6ccccc6cc45)cc3)c3ccc(-c4cccc5ccccc45)c(-c4ccccc4)c3)cc2)cc1 ; c1ccc(-c2ccc3ccccc3c2-c2ccc(N(c3ccccc3)c3ccc4c(c3)oc3cc5ccccc5cc34)cc2)cc1 ; c1ccc2cc3c(cc2c1)oc1ccc(N(c2ccc(-c4cccc5ccccc45)cc2)c2ccc(-c4cccc5ccccc45)cc2)cc13]

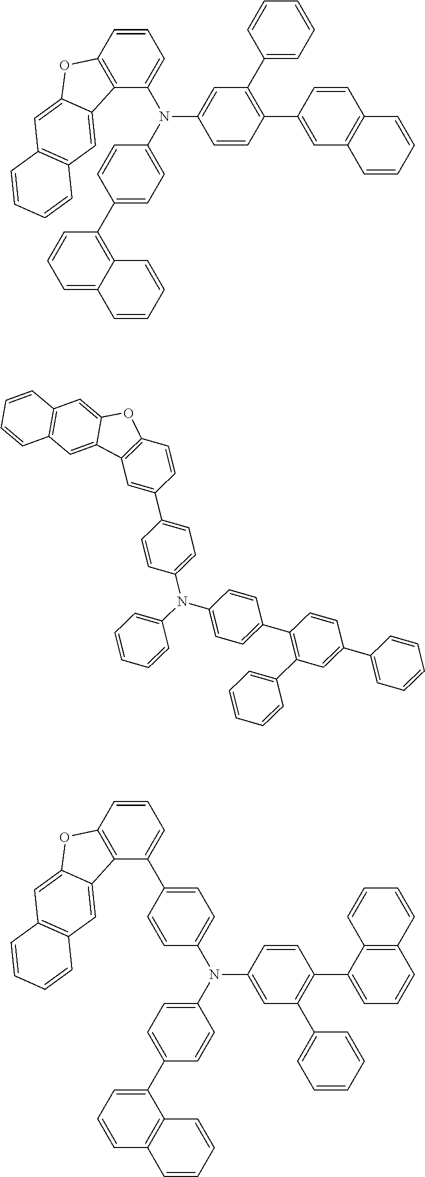 [SMILES: c1ccc(-c2cc(N(c3ccc(-c4cccc5ccccc45)cc3)c3cccc4oc5cc6ccccc6cc5c34)ccc2-c2ccc3ccccc3c2)cc1 ; c1ccc(-c2ccc(-c3ccc(N(c4ccccc4)c4ccc(-c5ccc6oc7cc8ccccc8cc7c6c5)cc4)cc3)c(-c3ccccc3)c2)cc1 ; c1ccc(-c2cc(N(c3ccc(-c4cccc5ccccc45)cc3)c3ccc(-c4cccc5oc6cc7ccccc7cc6c45)cc3)ccc2-c2cccc3ccccc23)cc1]

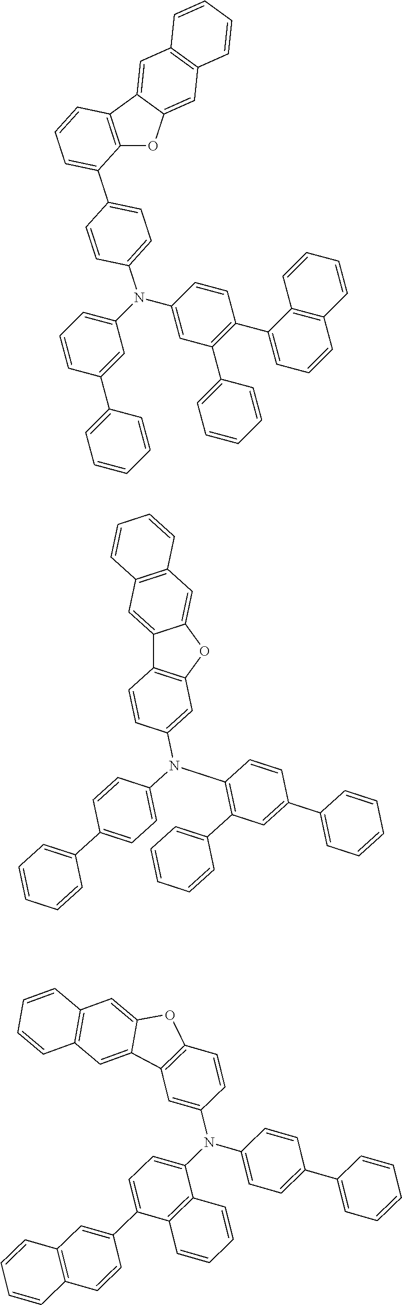 [SMILES: c1ccc(-c2cccc(N(c3ccc(-c4cccc5c4oc4cc6ccccc6cc45)cc3)c3ccc(-c4cccc5ccccc45)c(-c4ccccc4)c3)c2)cc1 ; c1ccc(-c2ccc(N(c3ccc4c(c3)oc3cc5ccccc5cc34)c3ccc(-c4ccccc4)cc3-c3ccccc3)cc2)cc1 ; c1ccc(-c2ccc(N(c3ccc4oc5cc6ccccc6cc5c4c3)c3ccc(-c4ccc5ccccc5c4)c4ccccc34)cc2)cc1]

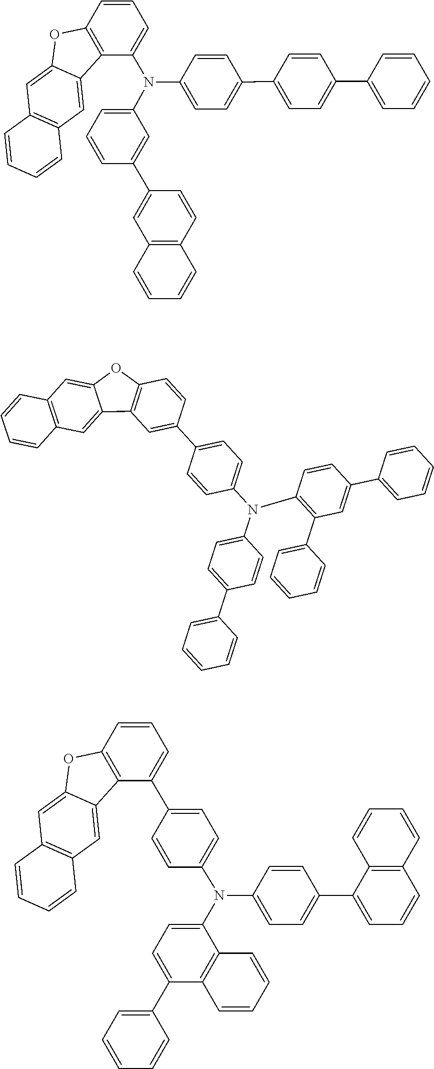 [SMILES: c1ccc(-c2ccc(-c3ccc(N(c4cccc(-c5ccc6ccccc6c5)c4)c4cccc5oc6cc7ccccc7cc6c45)cc3)cc2)cc1 ; c1ccc(-c2ccc(N(c3ccc(-c4ccc5oc6cc7ccccc7cc6c5c4)cc3)c3ccc(-c4ccccc4)cc3-c3ccccc3)cc2)cc1 ; c1ccc(-c2ccc(N(c3ccc(-c4cccc5ccccc45)cc3)c3ccc(-c4cccc5oc6cc7ccccc7cc6c45)cc3)c3ccccc23)cc1]

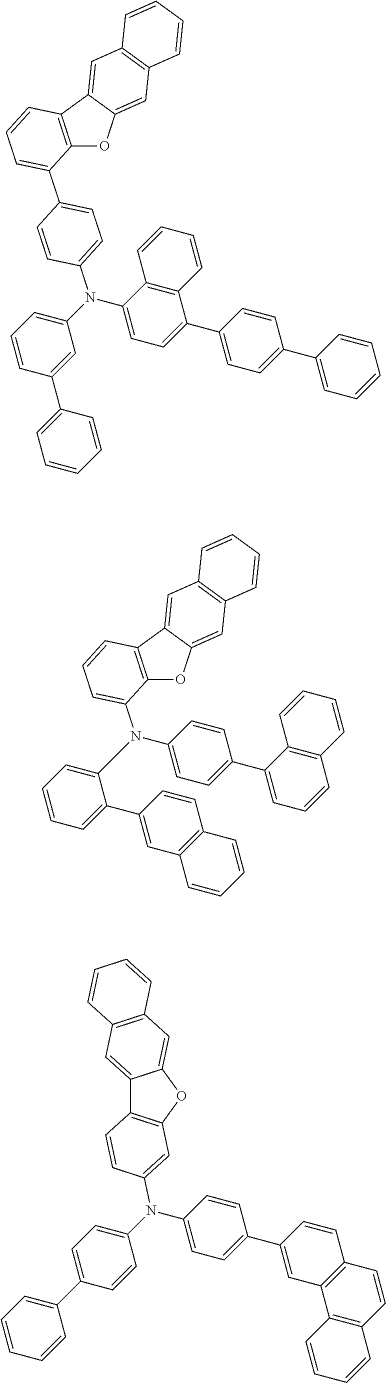 [SMILES: c1ccc(-c2ccc(-c3ccc(N(c4ccc(-c5cccc6c5oc5cc7ccccc7cc56)cc4)c4cccc(-c5ccccc5)c4)c4ccccc34)cc2)cc1 ; c1ccc(N(c2ccc(-c3cccc4ccccc34)cc2)c2cccc3c2oc2cc4ccccc4cc23)c(-c2ccc3ccccc3c2)c1 ; c1ccc(-c2ccc(N(c3ccc(-c4ccc5ccc6ccccc6c5c4)cc3)c3ccc4c(c3)oc3cc5ccccc5cc34)cc2)cc1]

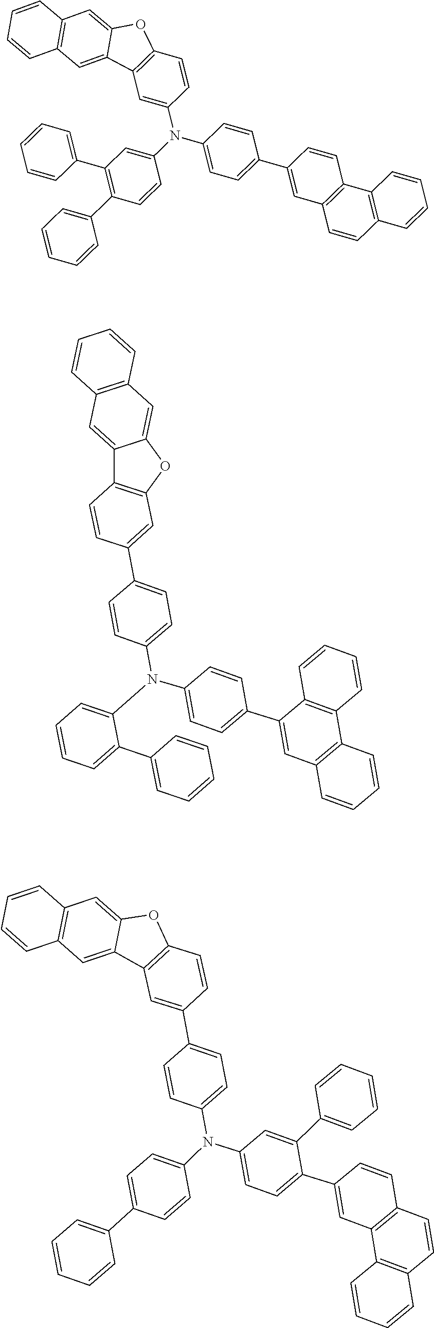 [SMILES: c1ccc(-c2ccc(N(c3ccc(-c4ccc5c(ccc6ccccc65)c4)cc3)c3ccc4oc5cc6ccccc6cc5c4c3)cc2-c2ccccc2)cc1 ; c1ccc(-c2ccccc2N(c2ccc(-c3ccc4c(c3)oc3cc5ccccc5cc34)cc2)c2ccc(-c3cc4ccccc4c4ccccc34)cc2)cc1 ; c1ccc(-c2ccc(N(c3ccc(-c4ccc5oc6cc7ccccc7cc6c5c4)cc3)c3ccc(-c4ccc5ccc6ccccc6c5c4)c(-c4ccccc4)c3)cc2)cc1]

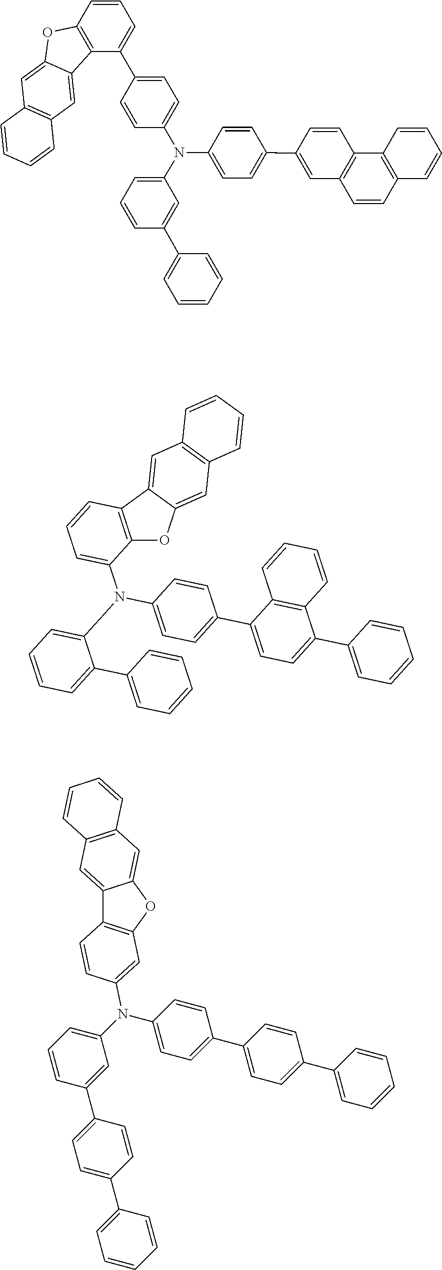 [SMILES: c1ccc(-c2cccc(N(c3ccc(-c4ccc5c(ccc6ccccc65)c4)cc3)c3ccc(-c4cccc5oc6cc7ccccc7cc6c45)cc3)c2)cc1 ; c1ccc(-c2ccccc2N(c2ccc(-c3ccc(-c4ccccc4)c4ccccc34)cc2)c2cccc3c2oc2cc4ccccc4cc23)cc1 ; c1ccc(-c2ccc(-c3ccc(N(c4cccc(-c5ccc(-c6ccccc6)cc5)c4)c4ccc5c(c4)oc4cc6ccccc6cc45)cc3)cc2)cc1]

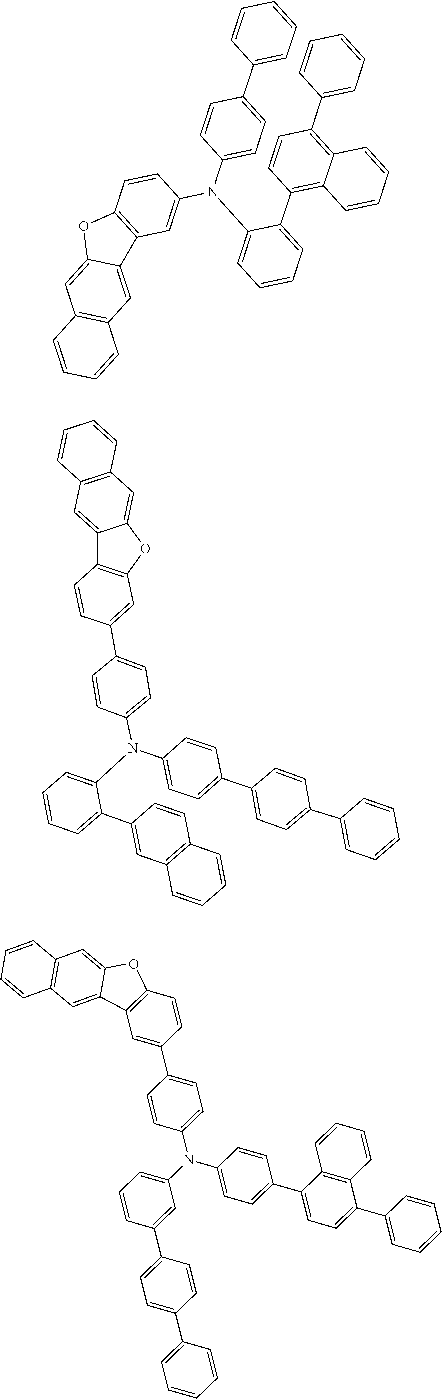

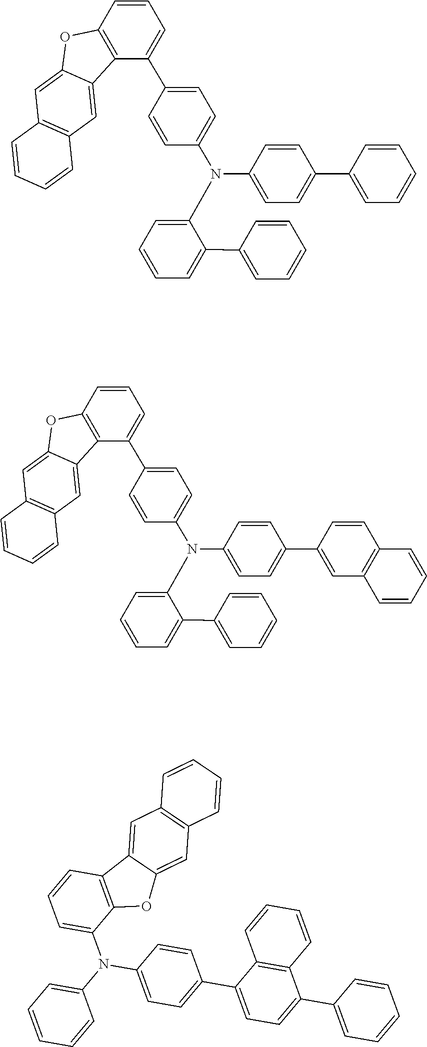 [SMILES: c1ccc(-c2ccc(N(c3ccc(-c4cccc5oc6cc7ccccc7cc6c45)cc3)c3ccccc3-c3ccccc3)cc2)cc1 ; c1ccc(-c2ccccc2N(c2ccc(-c3ccc4ccccc4c3)cc2)c2ccc(-c3cccc4oc5cc6ccccc6cc5c34)cc2)cc1 ; c1ccc(-c2ccc(-c3ccc(N(c4ccccc4)c4cccc5c4oc4cc6ccccc6cc45)cc3)c3ccccc23)cc1]

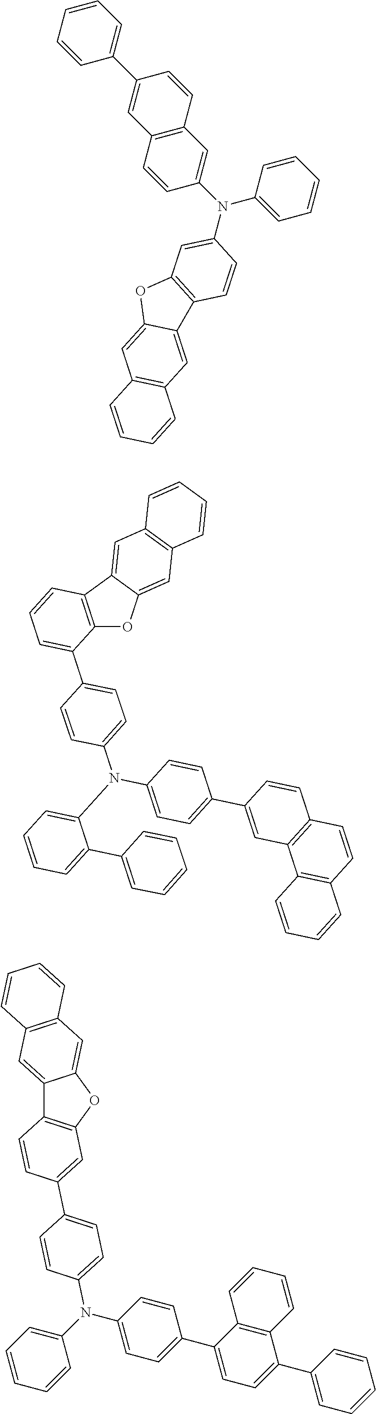

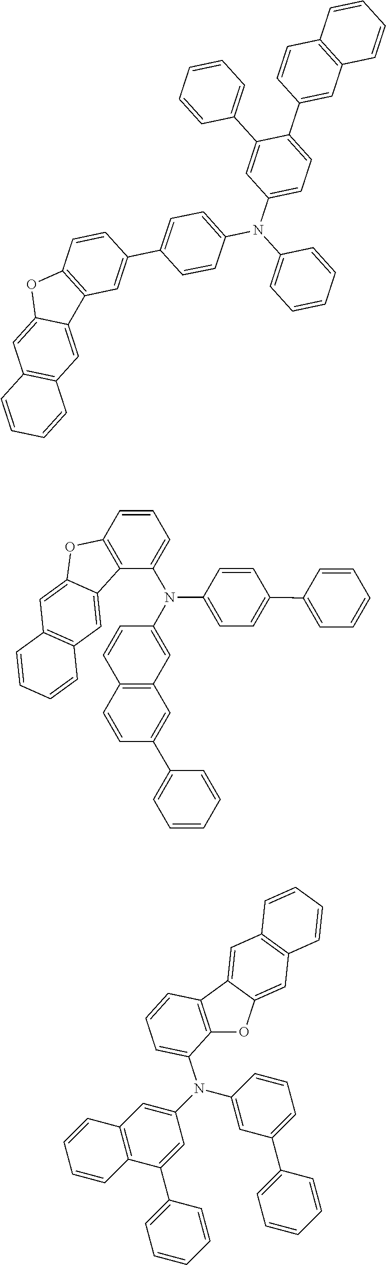 [SMILES: c1ccc(-c2cc(N(c3ccccc3)c3ccc(-c4ccc5oc6cc7ccccc7cc6c5c4)cc3)ccc2-c2ccc3ccccc3c2)cc1 ; c1ccc(-c2ccc(N(c3ccc4ccc(-c5ccccc5)cc4c3)c3cccc4oc5cc6ccccc6cc5c34)cc2)cc1 ; c1ccc(-c2cccc(N(c3cc(-c4ccccc4)c4ccccc4c3)c3cccc4c3oc3cc5ccccc5cc34)c2)cc1]

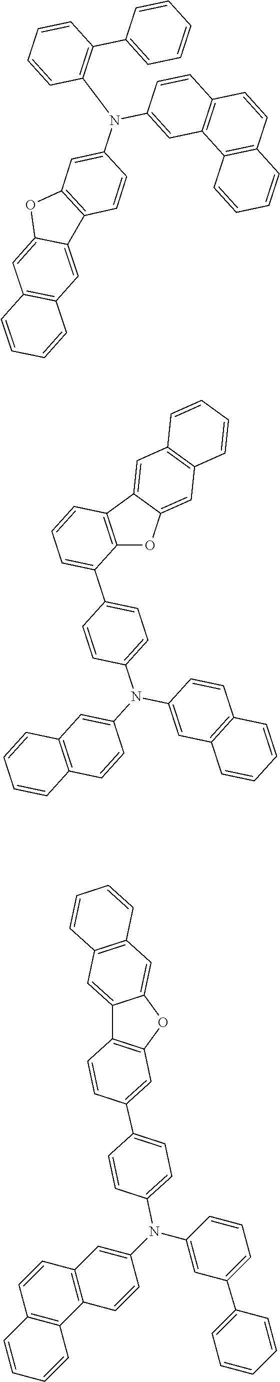 [SMILES: c1ccc(-c2ccccc2N(c2ccc3c(c2)oc2cc4ccccc4cc23)c2ccc3ccc4ccccc4c3c2)cc1 ; c1ccc2cc(N(c3ccc(-c4cccc5c4oc4cc6ccccc6cc45)cc3)c3ccc4ccccc4c3)ccc2c1 ; c1ccc(-c2cccc(N(c3ccc(-c4ccc5c(c4)oc4cc6ccccc6cc45)cc3)c3ccc4c(ccc5ccccc54)c3)c2)cc1]

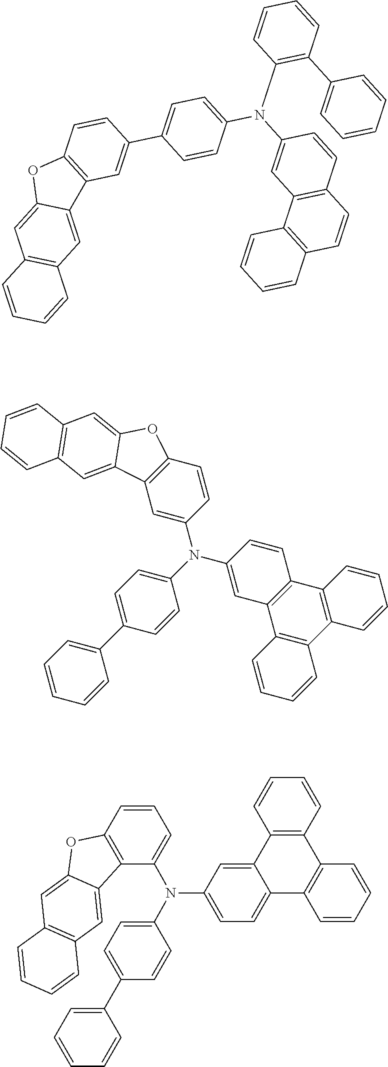 [SMILES: c1ccc(-c2ccccc2N(c2ccc(-c3ccc4oc5cc6ccccc6cc5c4c3)cc2)c2ccc3ccc4ccccc4c3c2)cc1 ; c1ccc(-c2ccc(N(c3ccc4oc5cc6ccccc6cc5c4c3)c3ccc4c5ccccc5c5ccccc5c4c3)cc2)cc1 ; c1ccc(-c2ccc(N(c3ccc4c5ccccc5c5ccccc5c4c3)c3cccc4oc5cc6ccccc6cc5c34)cc2)cc1]

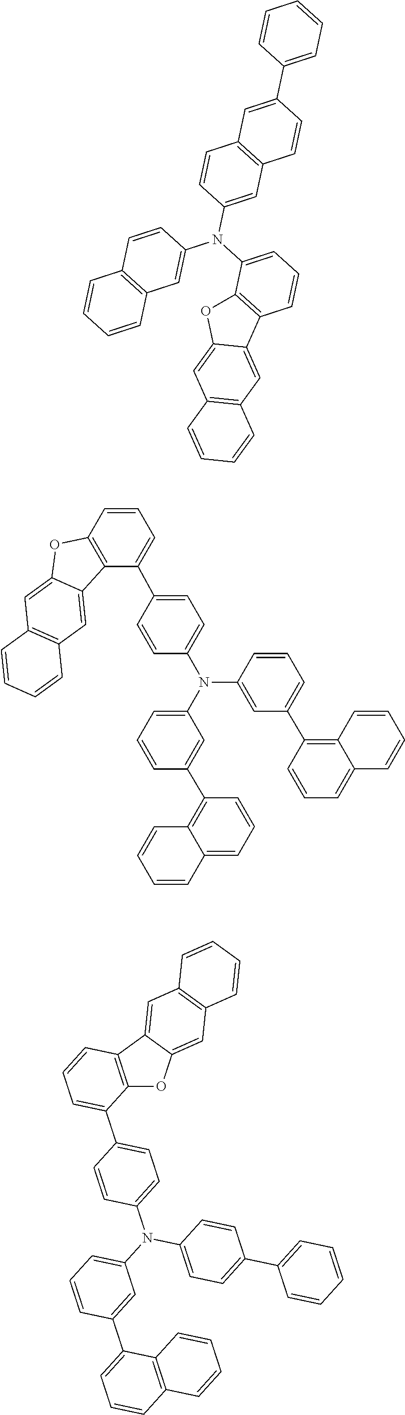 [SMILES: c1ccc(-c2ccc3cc(N(c4ccc5ccccc5c4)c4cccc5c4oc4cc6ccccc6cc45)ccc3c2)cc1 ; c1cc(-c2cccc3ccccc23)cc(N(c2ccc(-c3cccc4oc5cc6ccccc6cc5c34)cc2)c2cccc(-c3cccc4ccccc34)c2)c1 ; c1ccc(-c2ccc(N(c3ccc(-c4cccc5c4oc4cc6ccccc6cc45)cc3)c3cccc(-c4cccc5ccccc45)c3)cc2)cc1]

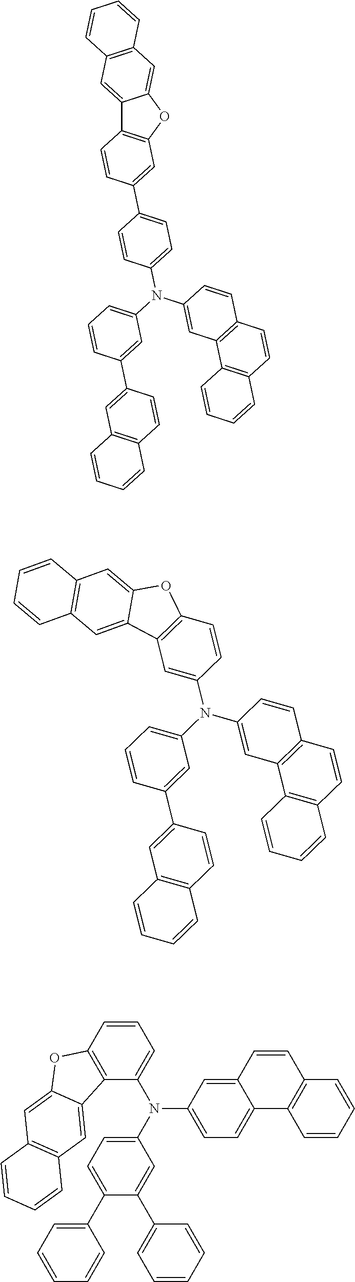 [SMILES: c1cc(-c2ccc3ccccc3c2)cc(N(c2ccc(-c3ccc4c(c3)oc3cc5ccccc5cc34)cc2)c2ccc3ccc4ccccc4c3c2)c1 ; c1cc(-c2ccc3ccccc3c2)cc(N(c2ccc3ccc4ccccc4c3c2)c2ccc3oc4cc5ccccc5cc4c3c2)c1 ; c1ccc(-c2ccc(N(c3ccc4c(ccc5ccccc54)c3)c3cccc4oc5cc6ccccc6cc5c34)cc2-c2ccccc2)cc1]

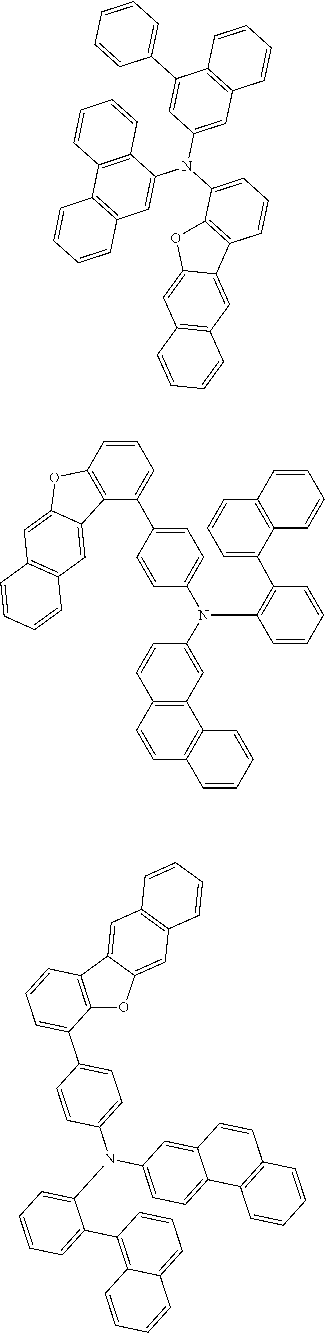 [SMILES: c1ccc(-c2cc(N(c3cc4ccccc4c4ccccc34)c3cccc4c3oc3cc5ccccc5cc34)cc3ccccc23)cc1 ; c1ccc(N(c2ccc(-c3cccc4oc5cc6ccccc6cc5c34)cc2)c2ccc3ccc4ccccc4c3c2)c(-c2cccc3ccccc23)c1 ; c1ccc(N(c2ccc(-c3cccc4c3oc3cc5ccccc5cc34)cc2)c2ccc3c(ccc4ccccc43)c2)c(-c2cccc3ccccc23)c1]

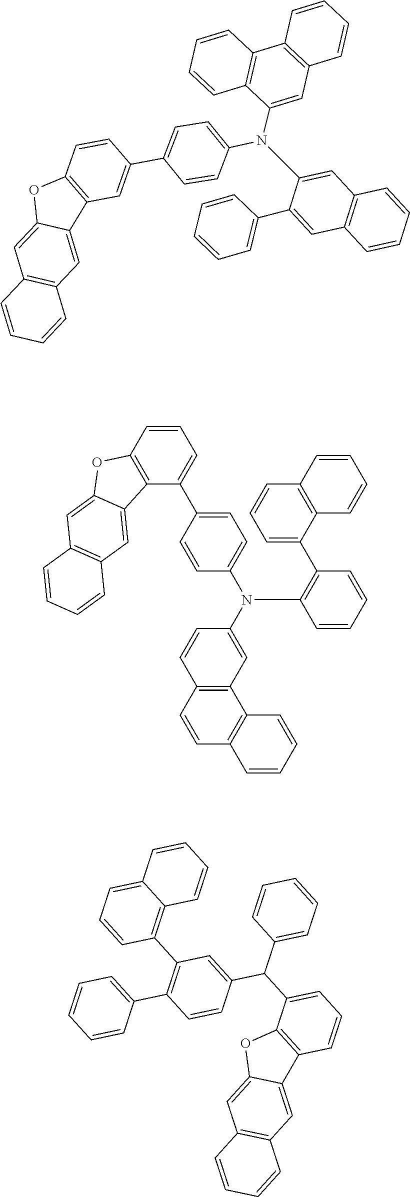 [SMILES: c1ccc(-c2cc3ccccc3cc2N(c2ccc(-c3ccc4oc5cc6ccccc6cc5c4c3)cc2)c2cc3ccccc3c3ccccc23)cc1 ; c1ccc(N(c2ccc(-c3cccc4oc5cc6ccccc6cc5c34)cc2)c2ccc3ccc4ccccc4c3c2)c(-c2cccc3ccccc23)c1 ; c1ccc(-c2ccc(C(c3ccccc3)c3cccc4c3oc3cc5ccccc5cc34)cc2-c2cccc3ccccc23)cc1]

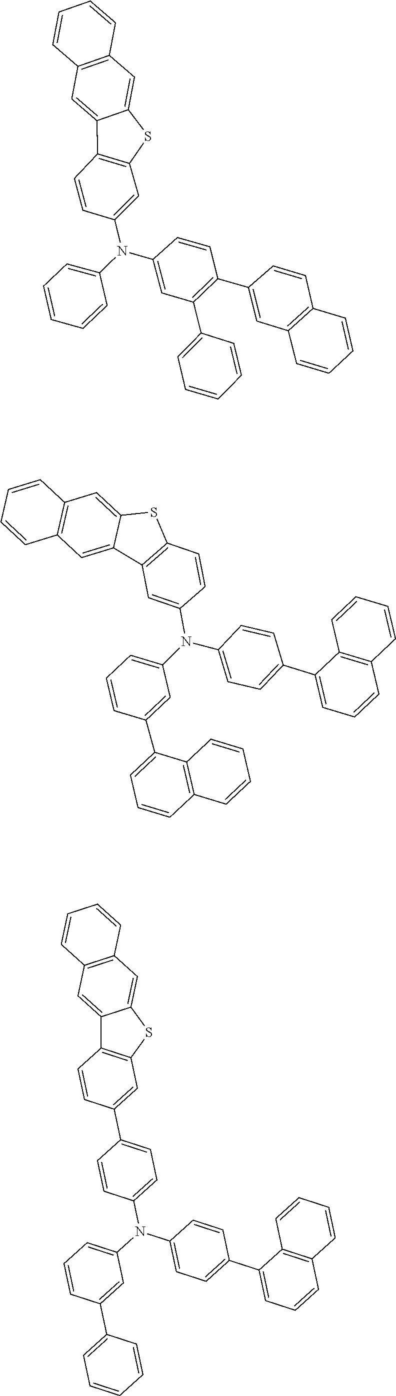 [SMILES: c1ccc(-c2cc(N(c3ccccc3)c3ccc4c(c3)sc3cc5ccccc5cc34)ccc2-c2ccc3ccccc3c2)cc1 ; c1cc(-c2cccc3ccccc23)cc(N(c2ccc(-c3cccc4ccccc34)cc2)c2ccc3sc4cc5ccccc5cc4c3c2)c1 ; c1ccc(-c2cccc(N(c3ccc(-c4ccc5c(c4)sc4cc6ccccc6cc45)cc3)c3ccc(-c4cccc5ccccc45)cc3)c2)cc1]

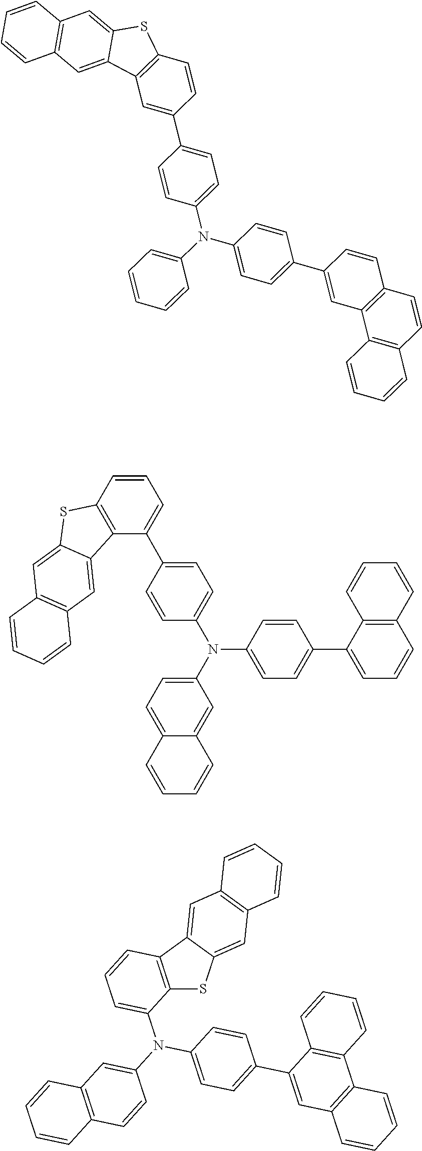 [SMILES: c1ccc(N(c2ccc(-c3ccc4ccc5ccccc5c4c3)cc2)c2ccc(-c3ccc4sc5cc6ccccc6cc5c4c3)cc2)cc1 ; c1ccc2cc(N(c3ccc(-c4cccc5ccccc45)cc3)c3ccc(-c4cccc5sc6cc7ccccc7cc6c45)cc3)ccc2c1 ; c1ccc2cc(N(c3ccc(-c4cc5ccccc5c5ccccc45)cc3)c3cccc4c3sc3cc5ccccc5cc34)ccc2c1]

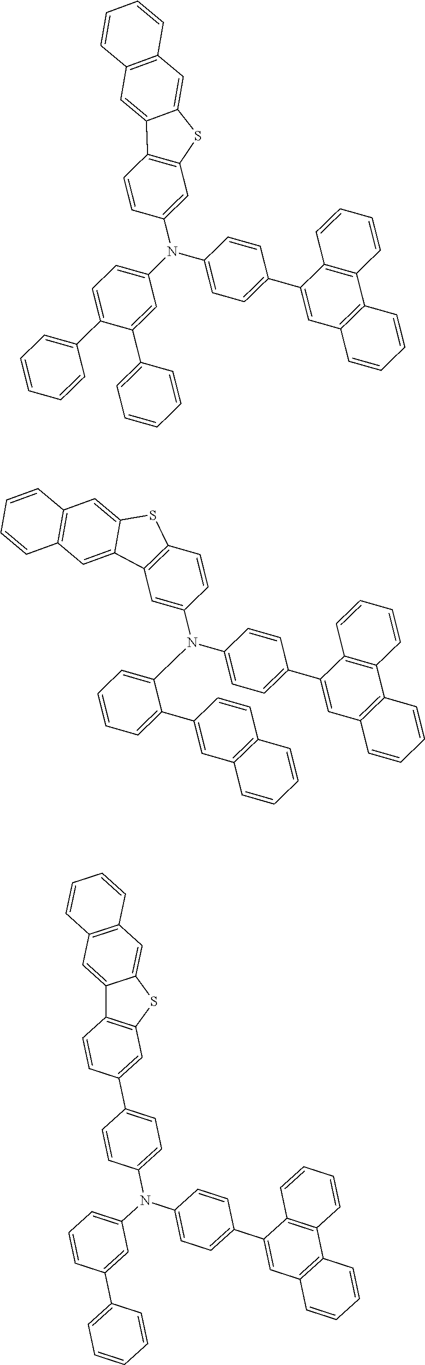 [SMILES: c1ccc(-c2ccc(N(c3ccc(-c4cc5ccccc5c5ccccc45)cc3)c3ccc4c(c3)sc3cc5ccccc5cc34)cc2-c2ccccc2)cc1 ; c1ccc(N(c2ccc(-c3cc4ccccc4c4ccccc34)cc2)c2ccc3sc4cc5ccccc5cc4c3c2)c(-c2ccc3ccccc3c2)c1 ; c1ccc(-c2cccc(N(c3ccc(-c4ccc5c(c4)sc4cc6ccccc6cc45)cc3)c3ccc(-c4cc5ccccc5c5ccccc45)cc3)c2)cc1]

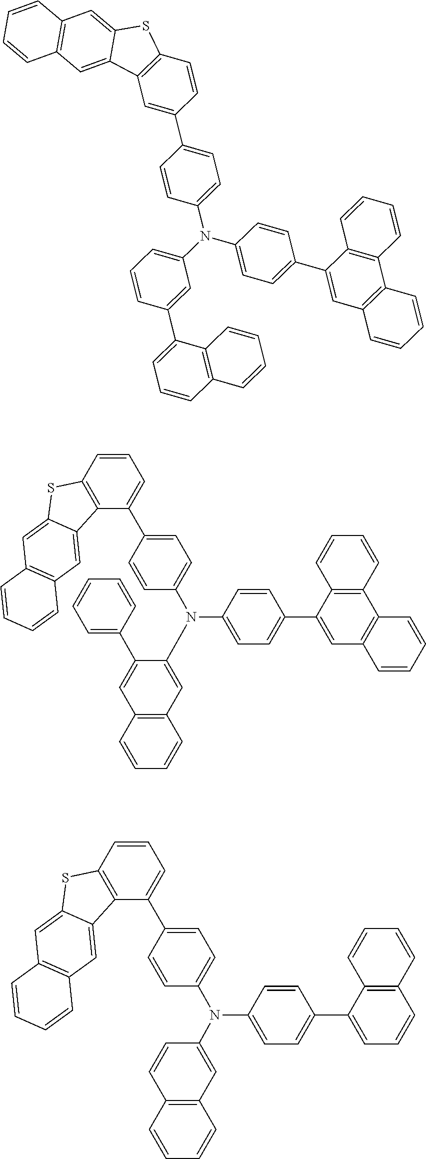 [SMILES: c1cc(-c2cccc3ccccc23)cc(N(c2ccc(-c3ccc4sc5cc6ccccc6cc5c4c3)cc2)c2ccc(-c3cc4ccccc4c4ccccc34)cc2)c1 ; c1ccc(-c2cc3ccccc3cc2N(c2ccc(-c3cc4ccccc4c4ccccc34)cc2)c2ccc(-c3cccc4sc5cc6ccccc6cc5c34)cc2)cc1 ; c1ccc2cc(N(c3ccc(-c4cccc5ccccc45)cc3)c3ccc(-c4cccc5sc6cc7ccccc7cc6c45)cc3)ccc2c1]

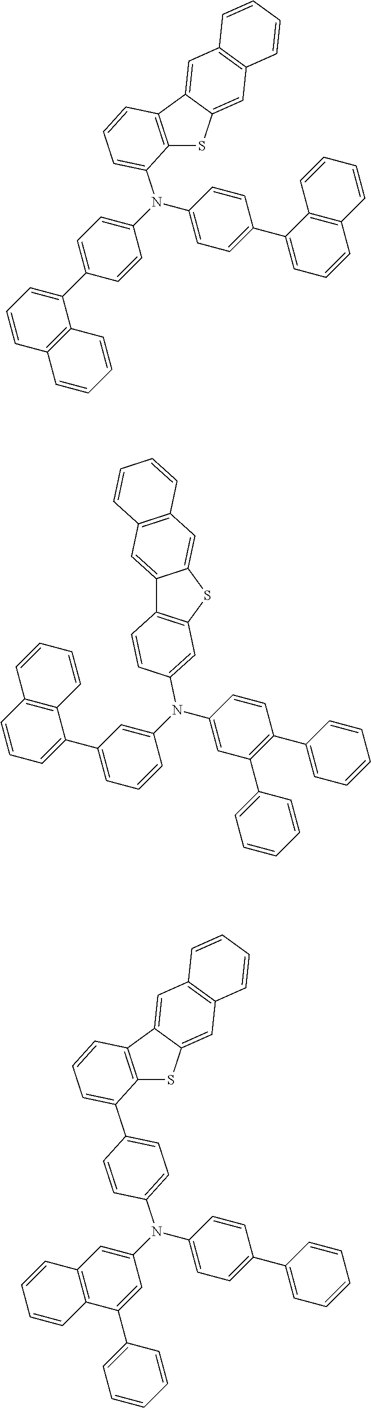 [SMILES: c1ccc2cc3c(cc2c1)sc1c(N(c2ccc(-c4cccc5ccccc45)cc2)c2ccc(-c4cccc5ccccc45)cc2)cccc13 ; c1ccc(-c2ccc(N(c3cccc(-c4cccc5ccccc45)c3)c3ccc4c(c3)sc3cc5ccccc5cc34)cc2-c2ccccc2)cc1 ; c1ccc(-c2ccc(N(c3ccc(-c4cccc5c4sc4cc6ccccc6cc45)cc3)c3cc(-c4ccccc4)c4ccccc4c3)cc2)cc1]

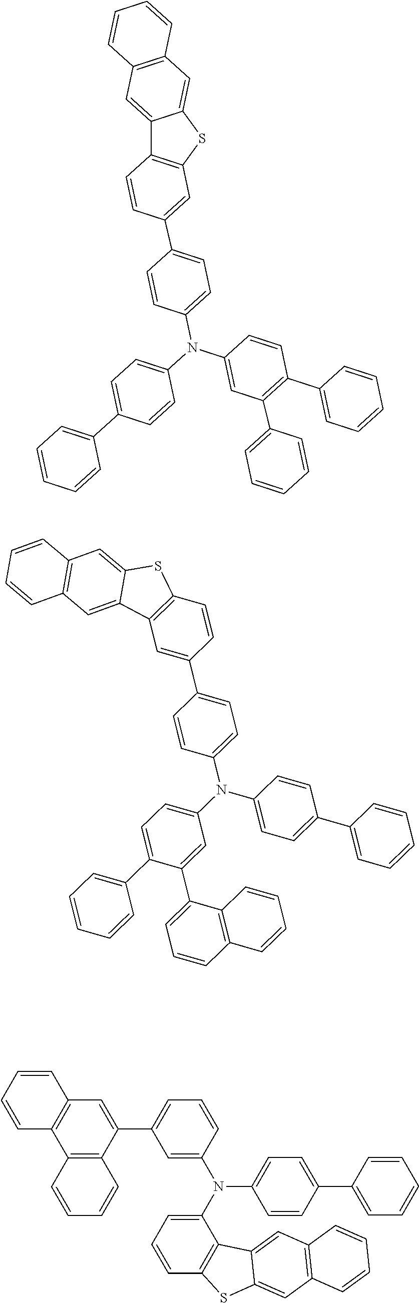 [SMILES: c1ccc(-c2ccc(N(c3ccc(-c4ccc5c(c4)sc4cc6ccccc6cc45)cc3)c3ccc(-c4ccccc4)c(-c4ccccc4)c3)cc2)cc1 ; c1ccc(-c2ccc(N(c3ccc(-c4ccc5sc6cc7ccccc7cc6c5c4)cc3)c3ccc(-c4ccccc4)c(-c4cccc5ccccc45)c3)cc2)cc1 ; c1ccc(-c2ccc(N(c3cccc(-c4cc5ccccc5c5ccccc45)c3)c3cccc4sc5cc6ccccc6cc5c34)cc2)cc1]

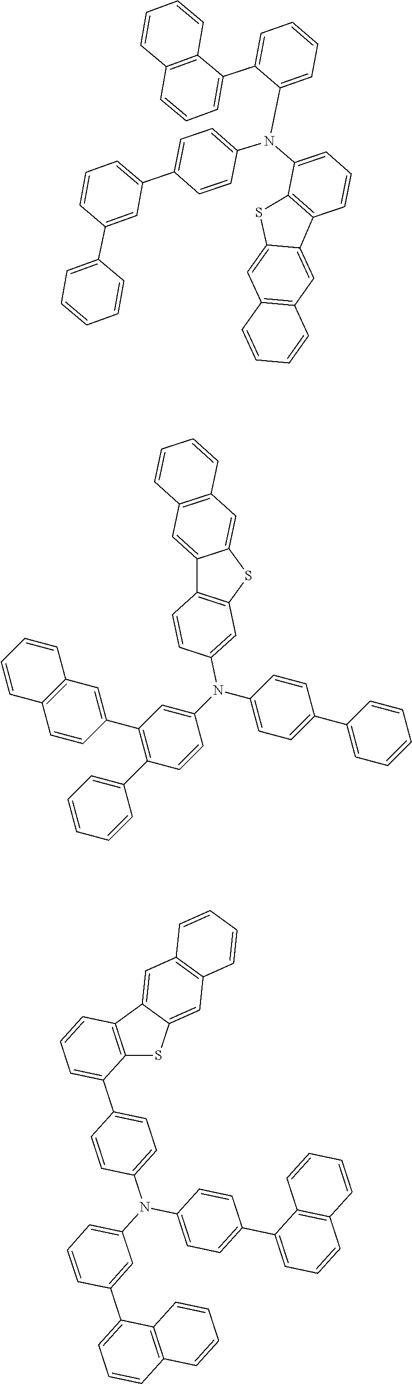 [SMILES: c1ccc(-c2cccc(-c3ccc(N(c4ccccc4-c4cccc5ccccc45)c4cccc5c4sc4cc6ccccc6cc45)cc3)c2)cc1 ; c1ccc(-c2ccc(N(c3ccc(-c4ccccc4)c(-c4ccc5ccccc5c4)c3)c3ccc4c(c3)sc3cc5ccccc5cc34)cc2)cc1 ; c1cc(-c2cccc3ccccc23)cc(N(c2ccc(-c3cccc4ccccc34)cc2)c2ccc(-c3cccc4c3sc3cc5ccccc5cc34)cc2)c1]

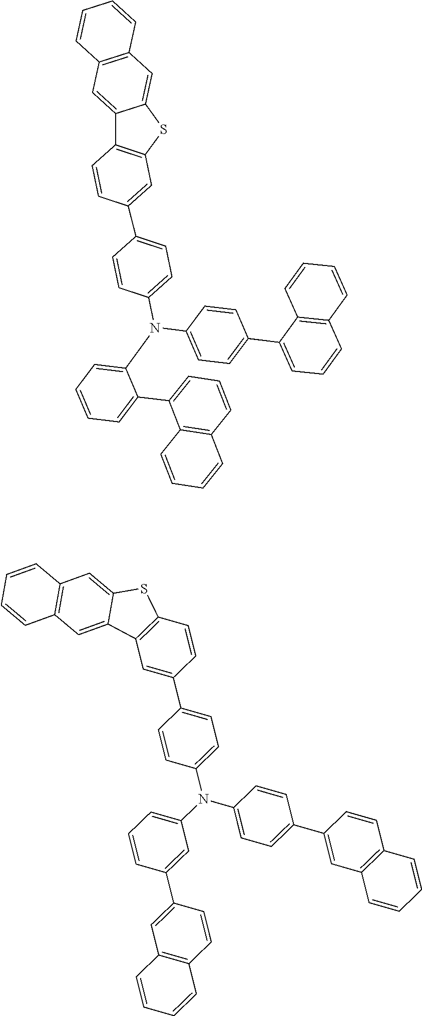 [SMILES: c1ccc(N(c2ccc(-c3ccc4c(c3)sc3cc5ccccc5cc34)cc2)c2ccc(-c3cccc4ccccc34)cc2)c(-c2cccc3ccccc23)c1 ; c1cc(-c2ccc3ccccc3c2)cc(N(c2ccc(-c3ccc4ccccc4c3)cc2)c2ccc(-c3ccc4sc5cc6ccccc6cc5c4c3)cc2)c1]

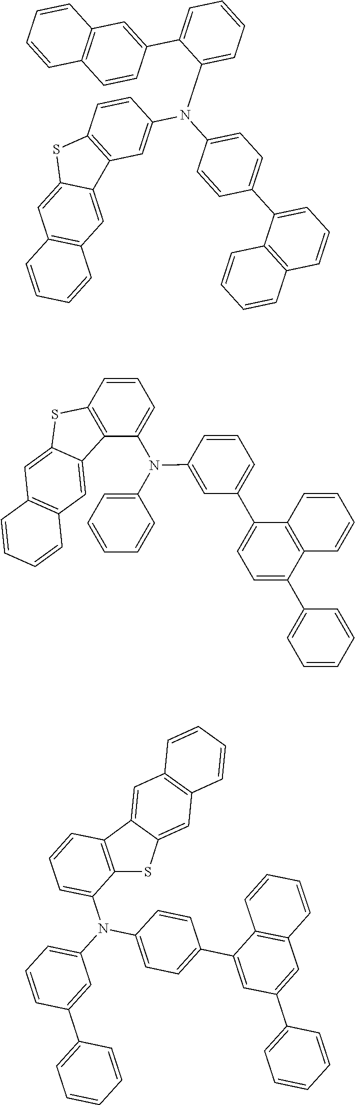 [SMILES: c1ccc(N(c2ccc(-c3cccc4ccccc34)cc2)c2ccc3sc4cc5ccccc5cc4c3c2)c(-c2ccc3ccccc3c2)c1 ; c1ccc(-c2ccc(-c3cccc(N(c4ccccc4)c4cccc5sc6cc7ccccc7cc6c45)c3)c3ccccc23)cc1 ; c1ccc(-c2cccc(N(c3ccc(-c4cc(-c5ccccc5)cc5ccccc45)cc3)c3cccc4c3sc3cc5ccccc5cc34)c2)cc1]

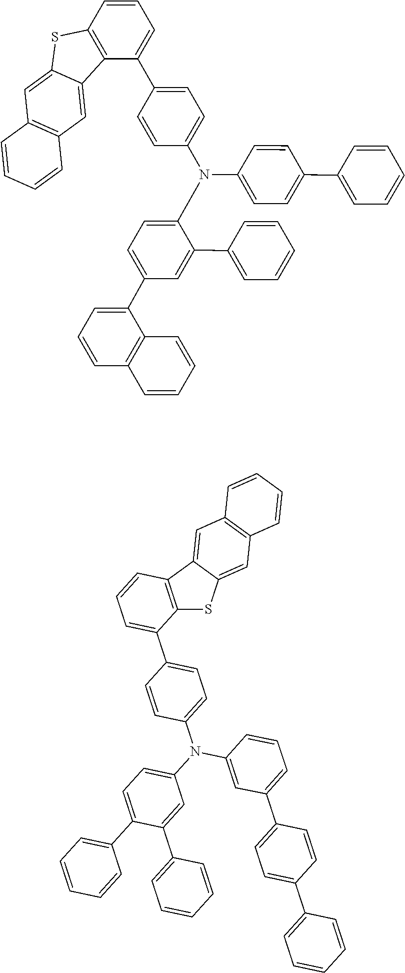 [SMILES: c1ccc(-c2ccc(N(c3ccc(-c4cccc5sc6cc7ccccc7cc6c45)cc3)c3ccc(-c4cccc5ccccc45)cc3-c3ccccc3)cc2)cc1 ; c1ccc(-c2ccc(-c3cccc(N(c4ccc(-c5cccc6c5sc5cc7ccccc7cc56)cc4)c4ccc(-c5ccccc5)c(-c5ccccc5)c4)c3)cc2)cc1]

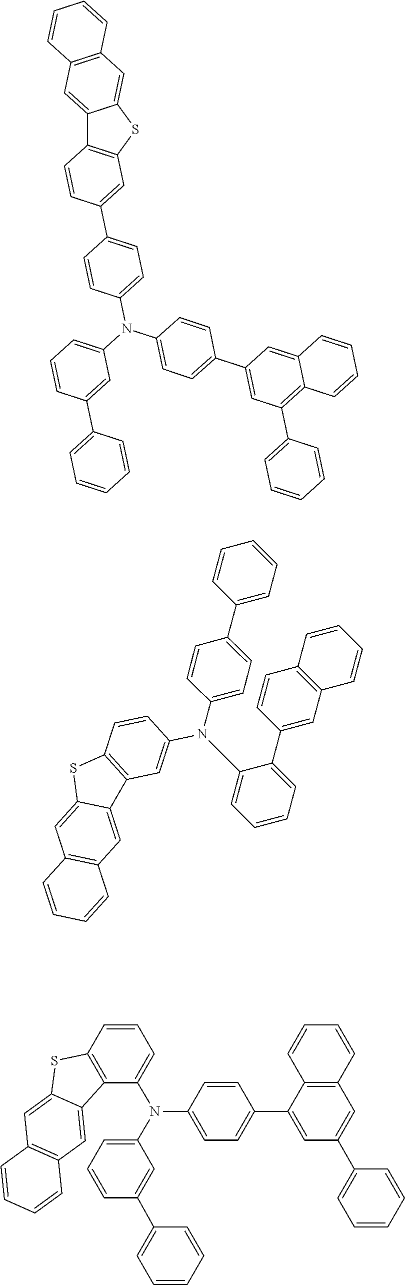 [SMILES: c1ccc(-c2cccc(N(c3ccc(-c4cc(-c5ccccc5)c5ccccc5c4)cc3)c3ccc(-c4ccc5c(c4)sc4cc6ccccc6cc45)cc3)c2)cc1 ; c1ccc(-c2ccc(N(c3ccc4sc5cc6ccccc6cc5c4c3)c3ccccc3-c3ccc4ccccc4c3)cc2)cc1 ; c1ccc(-c2cccc(N(c3ccc(-c4cc(-c5ccccc5)cc5ccccc45)cc3)c3cccc4sc5cc6ccccc6cc5c34)c2)cc1]

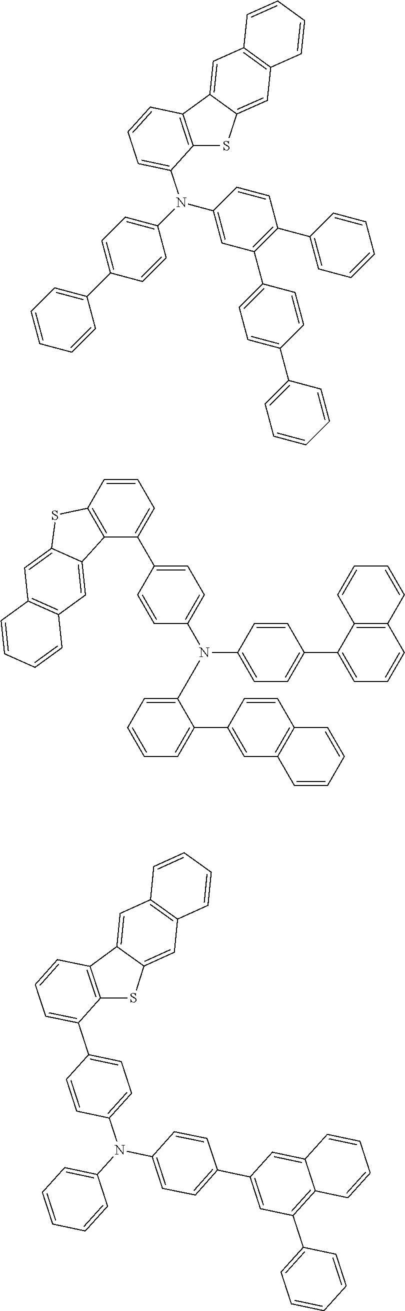 [SMILES: c1ccc(-c2ccc(-c3cc(N(c4ccc(-c5ccccc5)cc4)c4cccc5c4sc4cc6ccccc6cc45)ccc3-c3ccccc3)cc2)cc1 ; c1ccc(N(c2ccc(-c3cccc4ccccc34)cc2)c2ccc(-c3cccc4sc5cc6ccccc6cc5c34)cc2)c(-c2ccc3ccccc3c2)c1 ; c1ccc(-c2cc(-c3ccc(N(c4ccccc4)c4ccc(-c5cccc6c5sc5cc7ccccc7cc56)cc4)cc3)cc3ccccc23)cc1]

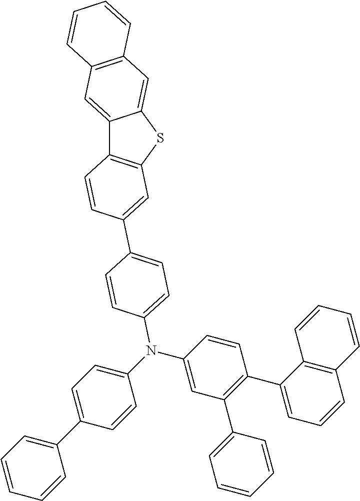 [SMILES: c1ccc(-c2ccc(N(c3ccc(-c4ccc5c(c4)sc4cc6ccccc6cc45)cc3)c3ccc(-c4cccc5ccccc45)c(-c4ccccc4)c3)cc2)cc1]

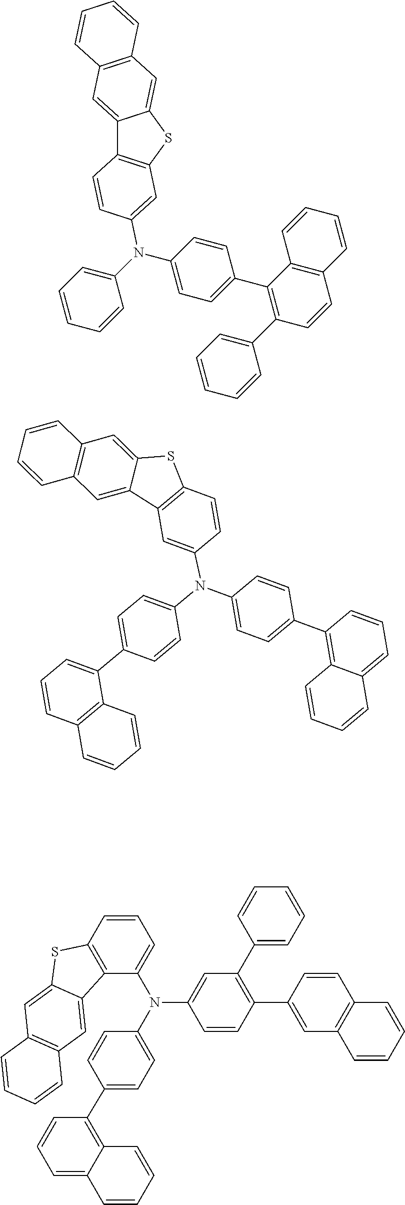 [SMILES: c1ccc(-c2ccc3ccccc3c2-c2ccc(N(c3ccccc3)c3ccc4c(c3)sc3cc5ccccc5cc34)cc2)cc1 ; c1ccc2cc3c(cc2c1)sc1ccc(N(c2ccc(-c4cccc5ccccc45)cc2)c2ccc(-c4cccc5ccccc45)cc2)cc13 ; c1ccc(-c2cc(N(c3ccc(-c4cccc5ccccc45)cc3)c3cccc4sc5cc6ccccc6cc5c34)ccc2-c2ccc3ccccc3c2)cc1]

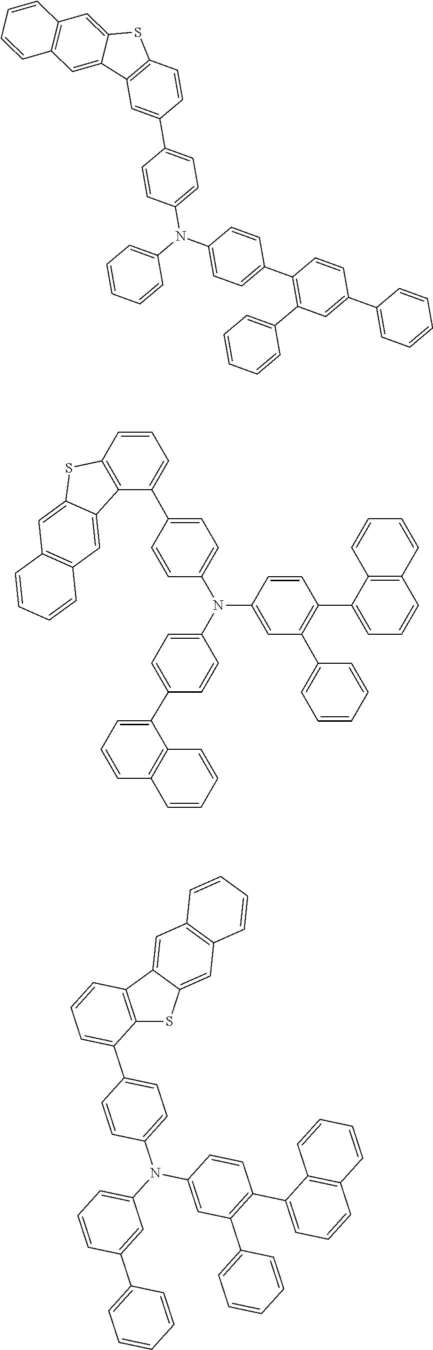 [SMILES: c1ccc(-c2ccc(-c3ccc(N(c4ccccc4)c4ccc(-c5ccc6sc7cc8ccccc8cc7c6c5)cc4)cc3)c(-c3ccccc3)c2)cc1 ; c1ccc(-c2cc(N(c3ccc(-c4cccc5ccccc45)cc3)c3ccc(-c4cccc5sc6cc7ccccc7cc6c45)cc3)ccc2-c2cccc3ccccc23)cc1 ; c1ccc(-c2cccc(N(c3ccc(-c4cccc5c4sc4cc6ccccc6cc45)cc3)c3ccc(-c4cccc5ccccc45)c(-c4ccccc4)c3)c2)cc1]

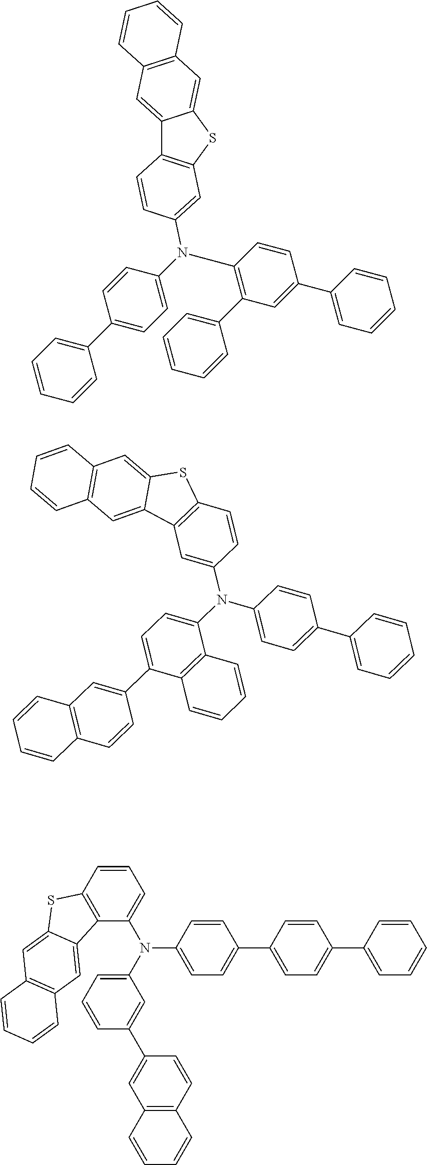

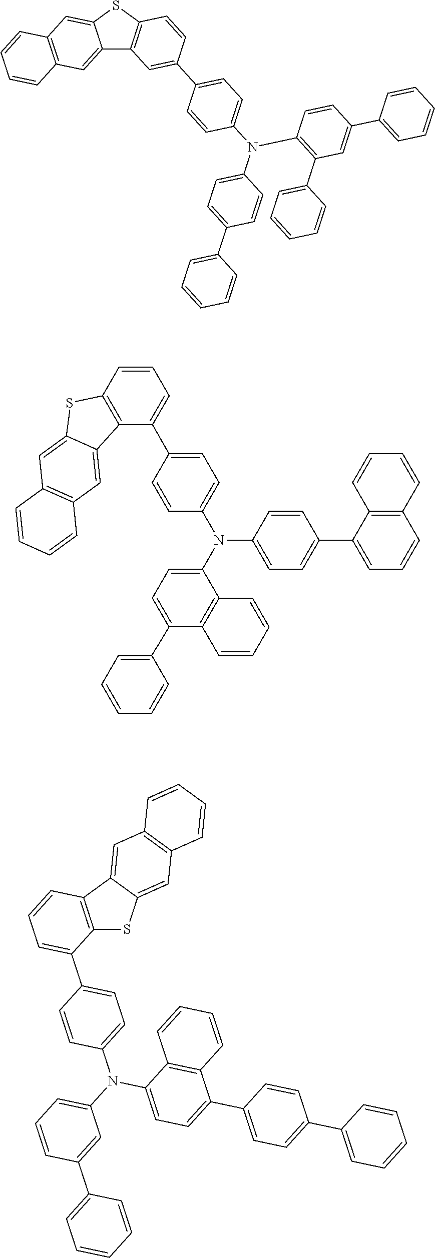 [SMILES: c1ccc(-c2ccc(N(c3ccc(-c4ccc5sc6cc7ccccc7cc6c5c4)cc3)c3ccc(-c4ccccc4)cc3-c3ccccc3)cc2)cc1 ; c1ccc(-c2ccc(N(c3ccc(-c4cccc5ccccc45)cc3)c3ccc(-c4cccc5sc6cc7ccccc7cc6c45)cc3)c3ccccc23)cc1 ; c1ccc(-c2ccc(-c3ccc(N(c4ccc(-c5cccc6c5sc5cc7ccccc7cc56)cc4)c4cccc(-c5ccccc5)c4)c4ccccc34)cc2)cc1]

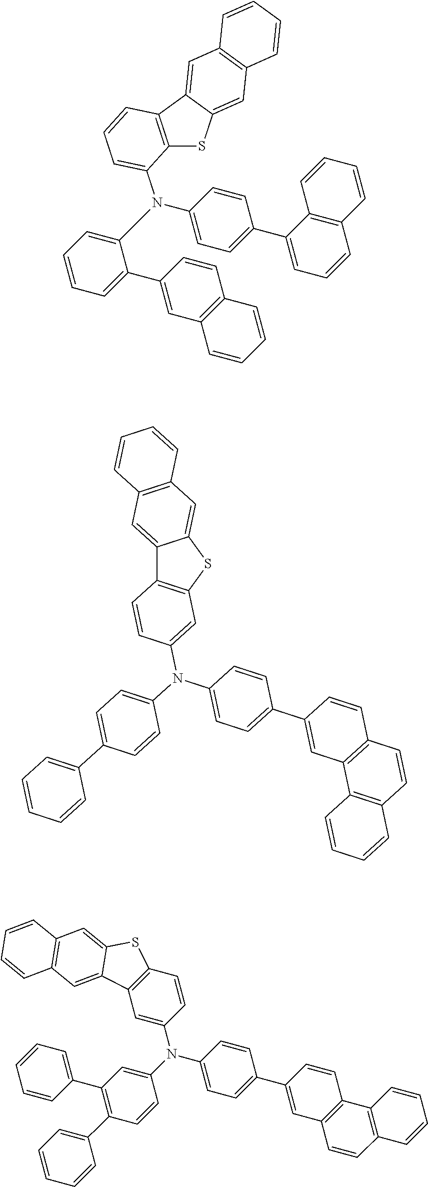 [SMILES: c1ccc(N(c2ccc(-c3cccc4ccccc34)cc2)c2cccc3c2sc2cc4ccccc4cc23)c(-c2ccc3ccccc3c2)c1 ; c1ccc(-c2ccc(N(c3ccc(-c4ccc5ccc6ccccc6c5c4)cc3)c3ccc4c(c3)sc3cc5ccccc5cc34)cc2)cc1 ; c1ccc(-c2ccc(N(c3ccc(-c4ccc5c(ccc6ccccc65)c4)cc3)c3ccc4sc5cc6ccccc6cc5c4c3)cc2-c2ccccc2)cc1]

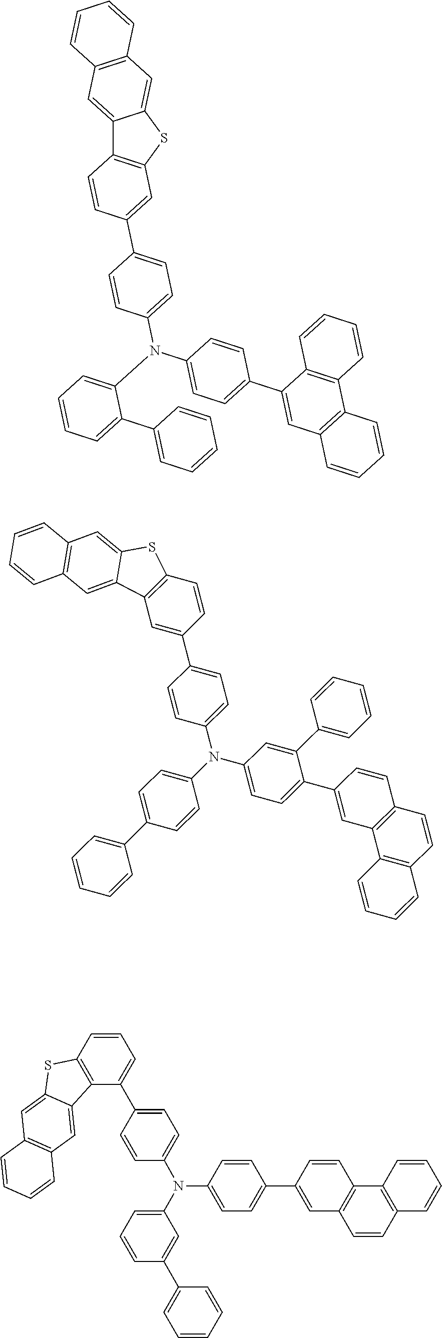 [SMILES: c1ccc(-c2ccccc2N(c2ccc(-c3ccc4c(c3)sc3cc5ccccc5cc34)cc2)c2ccc(-c3cc4ccccc4c4ccccc34)cc2)cc1 ; c1ccc(-c2ccc(N(c3ccc(-c4ccc5sc6cc7ccccc7cc6c5c4)cc3)c3ccc(-c4ccc5ccc6ccccc6c5c4)c(-c4ccccc4)c3)cc2)cc1 ; c1ccc(-c2cccc(N(c3ccc(-c4ccc5c(ccc6ccccc65)c4)cc3)c3ccc(-c4cccc5sc6cc7ccccc7cc6c45)cc3)c2)cc1]

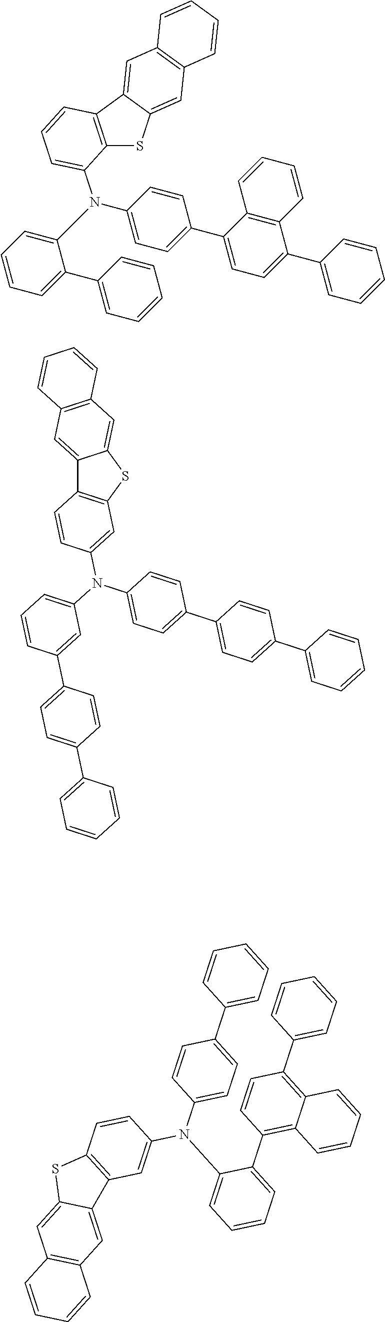 [SMILES: c1ccc(-c2ccccc2N(c2ccc(-c3ccc(-c4ccccc4)c4ccccc34)cc2)c2cccc3c2sc2cc4ccccc4cc23)cc1 ; c1ccc(-c2ccc(-c3ccc(N(c4cccc(-c5ccc(-c6ccccc6)cc5)c4)c4ccc5c(c4)sc4cc6ccccc6cc45)cc3)cc2)cc1 ; c1ccc(-c2ccc(N(c3ccc4sc5cc6ccccc6cc5c4c3)c3ccccc3-c3ccc(-c4ccccc4)c4ccccc34)cc2)cc1]

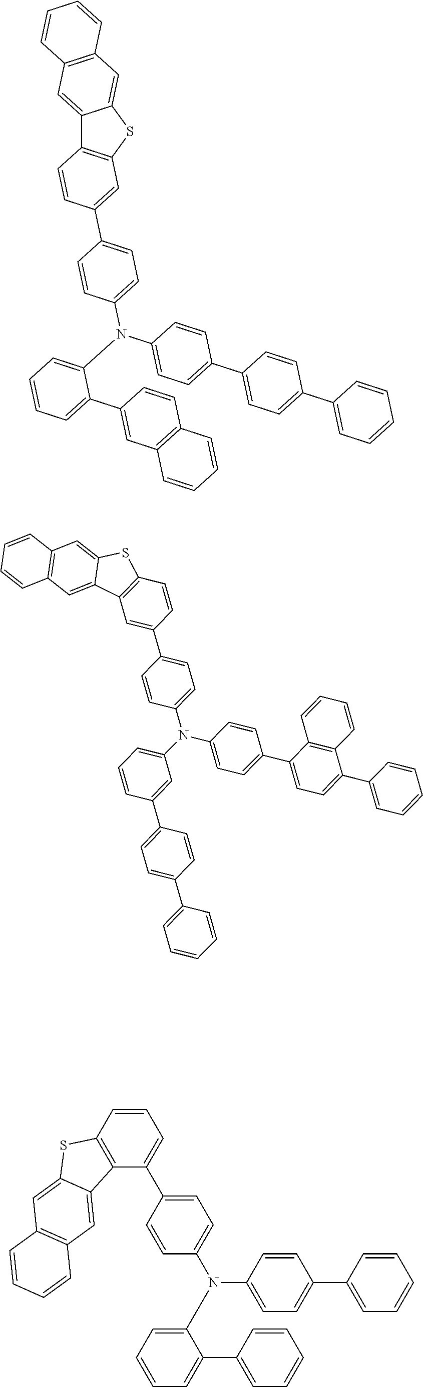 [SMILES: c1ccc(-c2ccc(-c3ccc(N(c4ccc(-c5ccc6c(c5)sc5cc7ccccc7cc56)cc4)c4ccccc4-c4ccc5ccccc5c4)cc3)cc2)cc1 ; c1ccc(-c2ccc(-c3cccc(N(c4ccc(-c5ccc6sc7cc8ccccc8cc7c6c5)cc4)c4ccc(-c5ccc(-c6ccccc6)c6ccccc56)cc4)c3)cc2)cc1 ; c1ccc(-c2ccc(N(c3ccc(-c4cccc5sc6cc7ccccc7cc6c45)cc3)c3ccccc3-c3ccccc3)cc2)cc1]

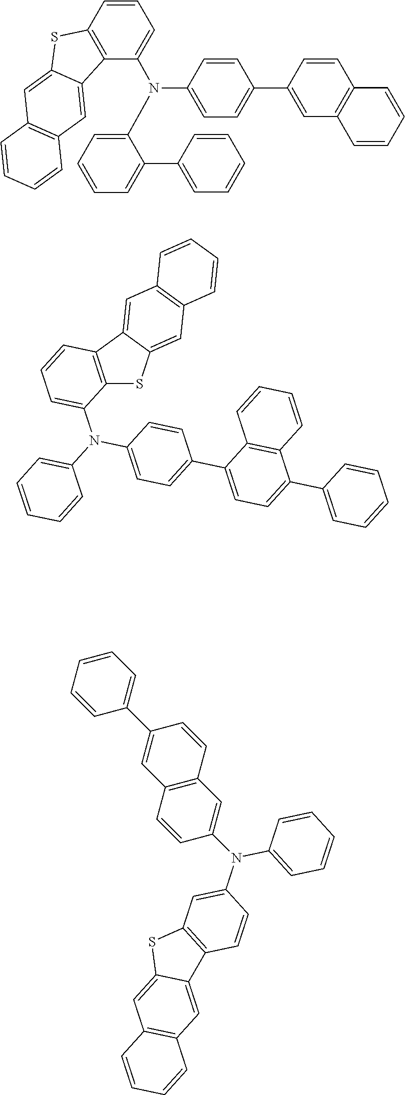 [SMILES: c1ccc(-c2ccccc2N(c2ccc(-c3ccc4ccccc4c3)cc2)c2cccc3sc4cc5ccccc5cc4c23)cc1 ; c1ccc(-c2ccc(-c3ccc(N(c4ccccc4)c4cccc5c4sc4cc6ccccc6cc45)cc3)c3ccccc23)cc1 ; c1ccc(-c2ccc3cc(N(c4ccccc4)c4ccc5c(c4)sc4cc6ccccc6cc45)ccc3c2)cc1]

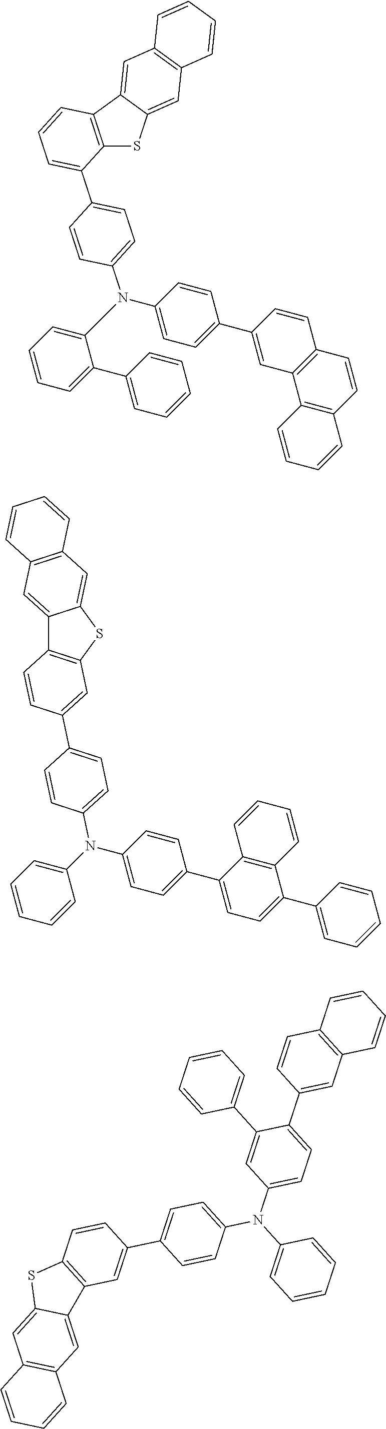

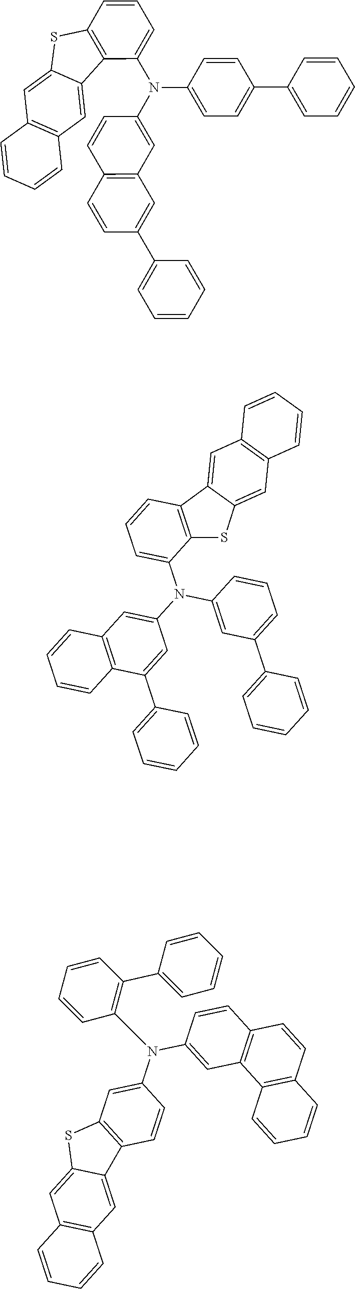 [SMILES: c1ccc(-c2ccc(N(c3ccc4ccc(-c5ccccc5)cc4c3)c3cccc4sc5cc6ccccc6cc5c34)cc2)cc1 ; c1ccc(-c2cccc(N(c3cc(-c4ccccc4)c4ccccc4c3)c3cccc4c3sc3cc5ccccc5cc34)c2)cc1 ; c1ccc(-c2ccccc2N(c2ccc3c(c2)sc2cc4ccccc4cc23)c2ccc3ccc4ccccc4c3c2)cc1]

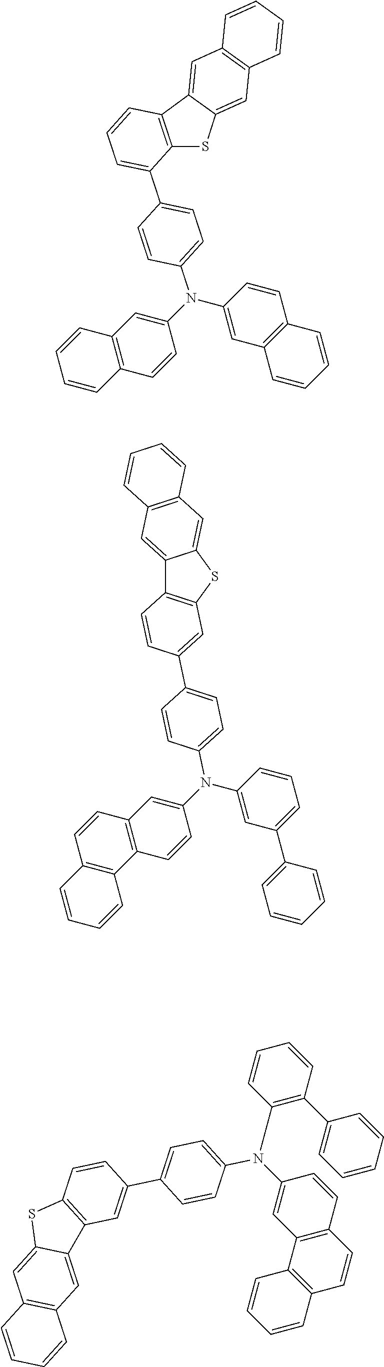 [SMILES: c1ccc2cc(N(c3ccc(-c4cccc5c4sc4cc6ccccc6cc45)cc3)c3ccc4ccccc4c3)ccc2c1 ; c1ccc(-c2cccc(N(c3ccc(-c4ccc5c(c4)sc4cc6ccccc6cc45)cc3)c3ccc4c(ccc5ccccc54)c3)c2)cc1 ; c1ccc(-c2ccccc2N(c2ccc(-c3ccc4sc5cc6ccccc6cc5c4c3)cc2)c2ccc3ccc4ccccc4c3c2)cc1]

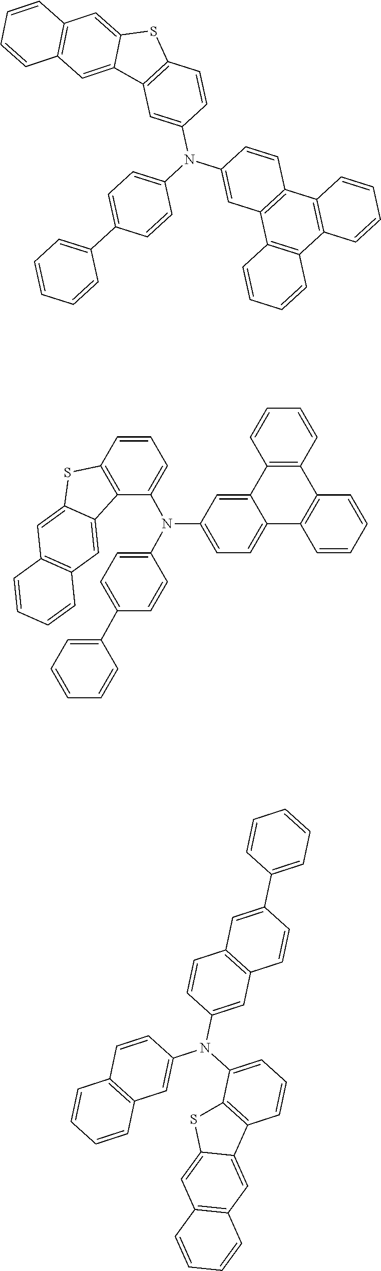 [SMILES: c1ccc(-c2ccc(N(c3ccc4sc5cc6ccccc6cc5c4c3)c3ccc4c5ccccc5c5ccccc5c4c3)cc2)cc1 ; c1ccc(-c2ccc(N(c3ccc4c5ccccc5c5ccccc5c4c3)c3cccc4sc5cc6ccccc6cc5c34)cc2)cc1 ; c1ccc(-c2ccc3cc(N(c4ccc5ccccc5c4)c4cccc5c4sc4cc6ccccc6cc45)ccc3c2)cc1]

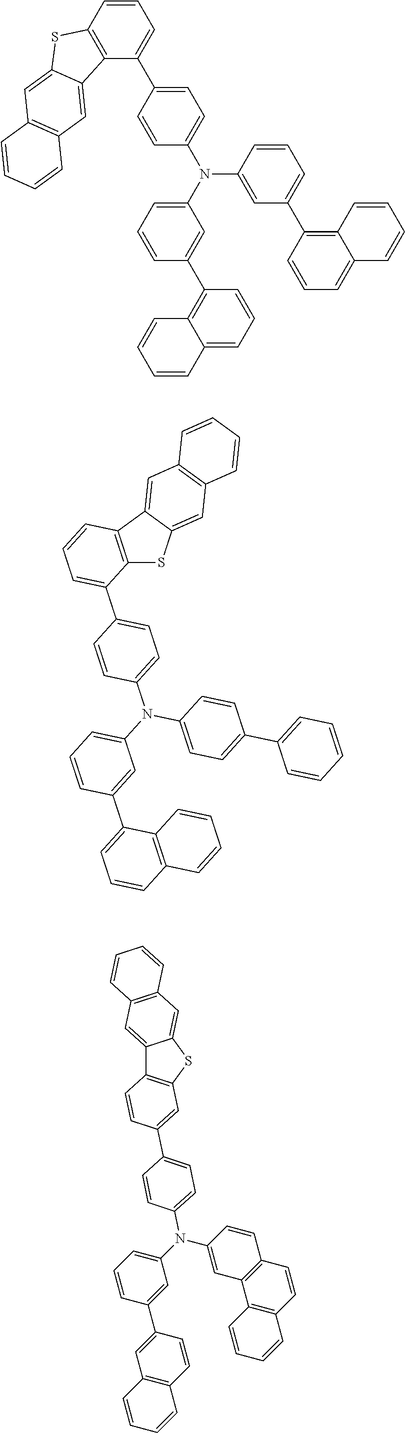 [SMILES: c1cc(-c2cccc3ccccc23)cc(N(c2ccc(-c3cccc4sc5cc6ccccc6cc5c34)cc2)c2cccc(-c3cccc4ccccc34)c2)c1 ; c1ccc(-c2ccc(N(c3ccc(-c4cccc5c4sc4cc6ccccc6cc45)cc3)c3cccc(-c4cccc5ccccc45)c3)cc2)cc1 ; c1cc(-c2ccc3ccccc3c2)cc(N(c2ccc(-c3ccc4c(c3)sc3cc5ccccc5cc34)cc2)c2ccc3ccc4ccccc4c3c2)c1]

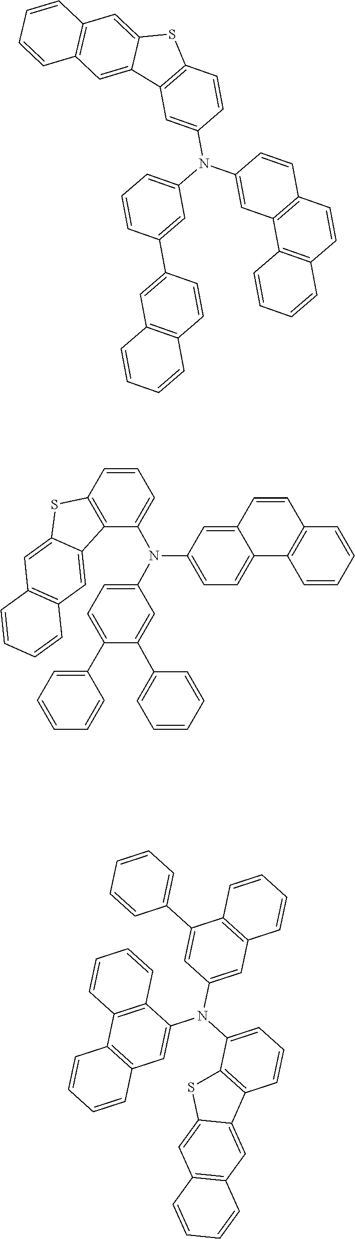 [SMILES: c1cc(-c2ccc3ccccc3c2)cc(N(c2ccc3ccc4ccccc4c3c2)c2ccc3sc4cc5ccccc5cc4c3c2)c1 ; c1ccc(-c2ccc(N(c3ccc4c(ccc5ccccc54)c3)c3cccc4sc5cc6ccccc6cc5c34)cc2-c2ccccc2)cc1 ; c1ccc(-c2cc(N(c3cc4ccccc4c4ccccc34)c3cccc4c3sc3cc5ccccc5cc34)cc3ccccc23)cc1]

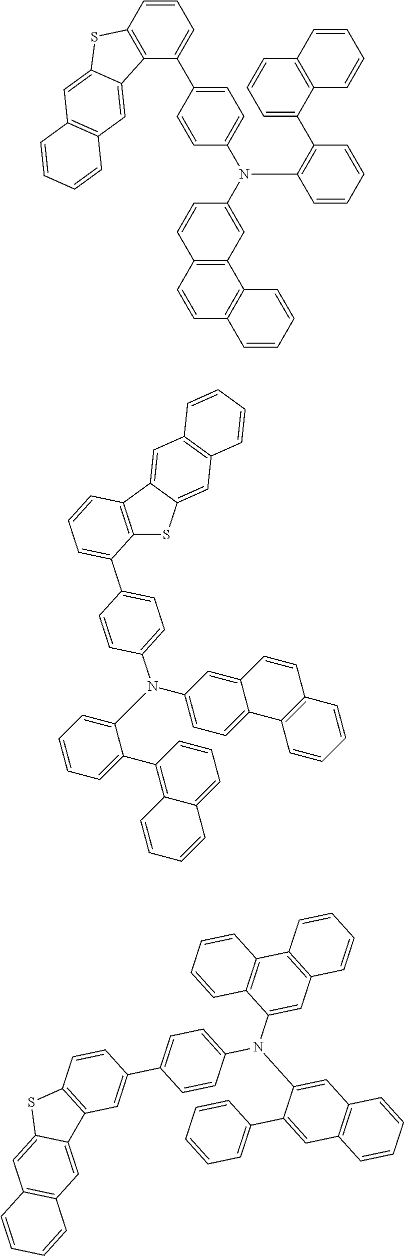 [SMILES: c1ccc(N(c2ccc(-c3cccc4sc5cc6ccccc6cc5c34)cc2)c2ccc3ccc4ccccc4c3c2)c(-c2cccc3ccccc23)c1 ; c1ccc(N(c2ccc(-c3cccc4c3sc3cc5ccccc5cc34)cc2)c2ccc3c(ccc4ccccc43)c2)c(-c2cccc3ccccc23)c1 ; c1ccc(-c2cc3ccccc3cc2N(c2ccc(-c3ccc4sc5cc6ccccc6cc5c4c3)cc2)c2cc3ccccc3c3ccccc23)cc1]

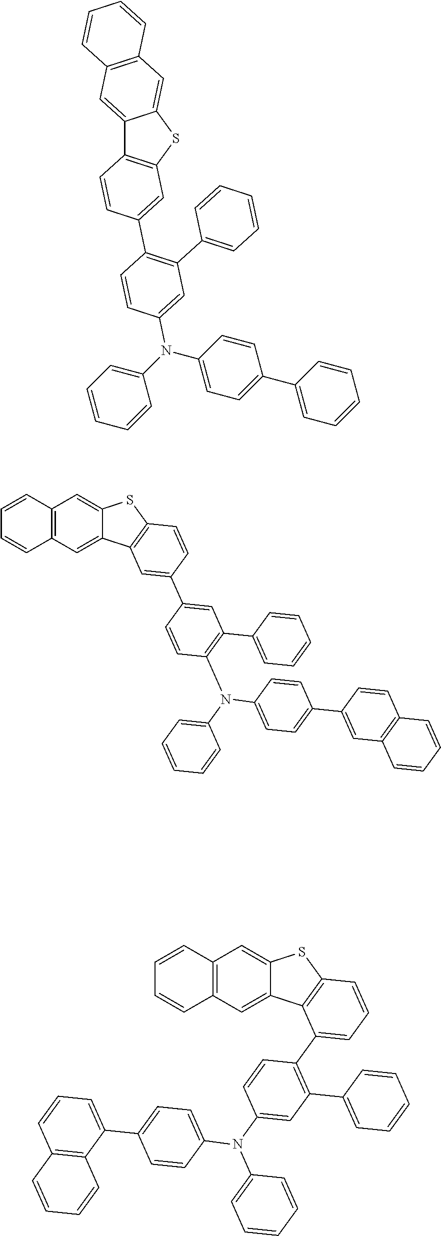 [SMILES: c1ccc(-c2ccc(N(c3ccccc3)c3ccc(-c4ccc5c(c4)sc4cc6ccccc6cc45)c(-c4ccccc4)c3)cc2)cc1 ; c1ccc(-c2cc(-c3ccc4sc5cc6ccccc6cc5c4c3)ccc2N(c2ccccc2)c2ccc(-c3ccc4ccccc4c3)cc2)cc1 ; c1ccc(-c2cc(N(c3ccccc3)c3ccc(-c4cccc5ccccc45)cc3)ccc2-c2cccc3sc4cc5ccccc5cc4c23)cc1]

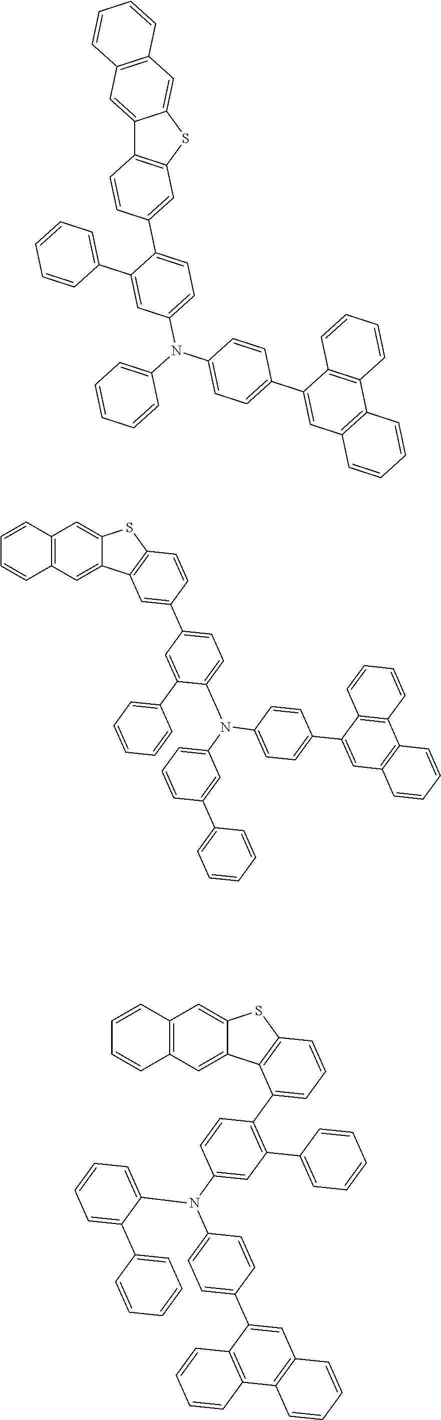 [SMILES: c1ccc(-c2cc(N(c3ccccc3)c3ccc(-c4cc5ccccc5c5ccccc45)cc3)ccc2-c2ccc3c(c2)sc2cc4ccccc4cc23)cc1 ; c1ccc(-c2cccc(N(c3ccc(-c4cc5ccccc5c5ccccc45)cc3)c3ccc(-c4ccc5sc6cc7ccccc7cc6c5c4)cc3-c3ccccc3)c2)cc1 ; c1ccc(-c2cc(N(c3ccc(-c4cc5ccccc5c5ccccc45)cc3)c3ccccc3-c3ccccc3)ccc2-c2cccc3sc4cc5ccccc5cc4c23)cc1]

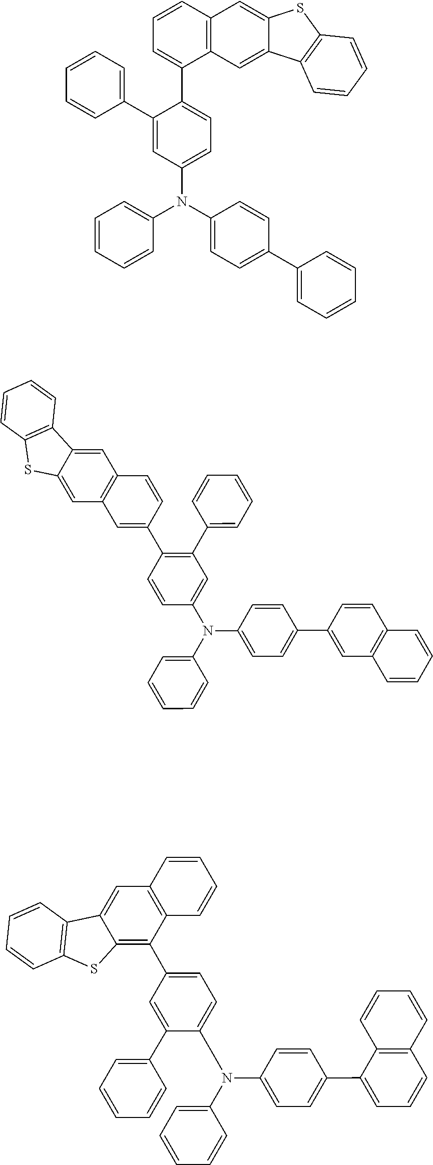 [SMILES: c1ccc(-c2ccc(N(c3ccccc3)c3ccc(-c4cccc5cc6sc7ccccc7c6cc45)c(-c4ccccc4)c3)cc2)cc1 ; c1ccc(-c2cc(N(c3ccccc3)c3ccc(-c4ccc5ccccc5c4)cc3)ccc2-c2ccc3cc4c(cc3c2)sc2ccccc24)cc1 ; c1ccc(-c2cc(-c3c4ccccc4cc4c3sc3ccccc34)ccc2N(c2ccccc2)c2ccc(-c3cccc4ccccc34)cc2)cc1]

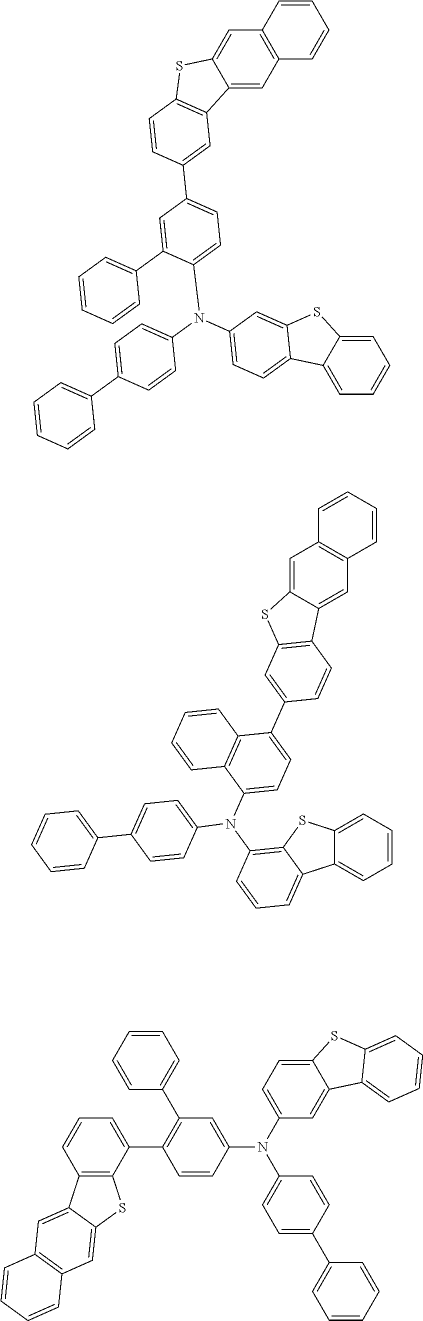 [SMILES: c1ccc(-c2ccc(N(c3ccc4c(c3)sc3ccccc34)c3ccc(-c4ccc5sc6cc7ccccc7cc6c5c4)cc3-c3ccccc3)cc2)cc1 ; c1ccc(-c2ccc(N(c3ccc(-c4ccc5c(c4)sc4cc6ccccc6cc45)c4ccccc34)c3cccc4c3sc3ccccc34)cc2)cc1 ; c1ccc(-c2ccc(N(c3ccc(-c4cccc5c4sc4cc6ccccc6cc45)c(-c4ccccc4)c3)c3ccc4sc5ccccc5c4c3)cc2)cc1]

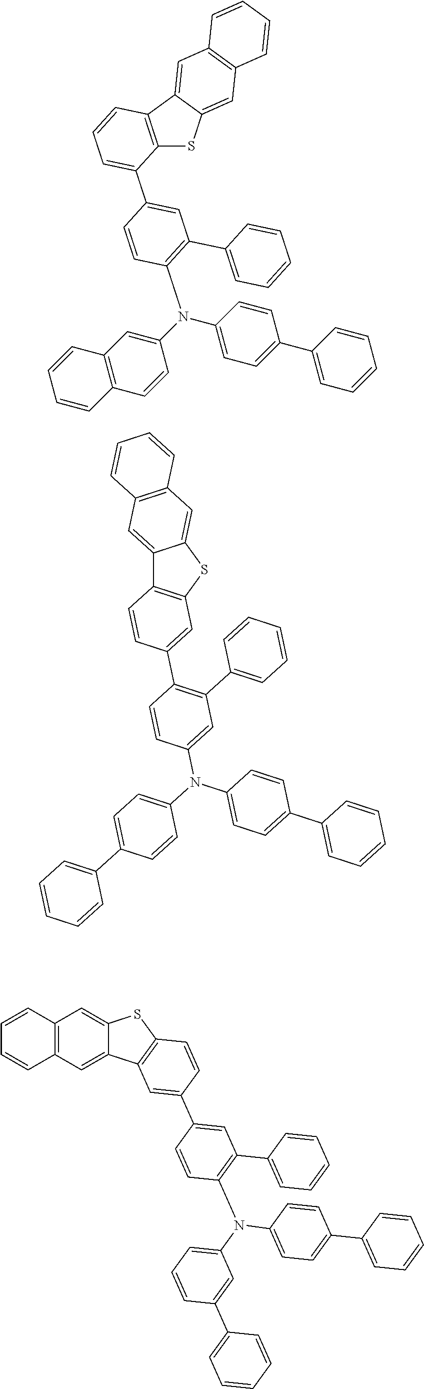

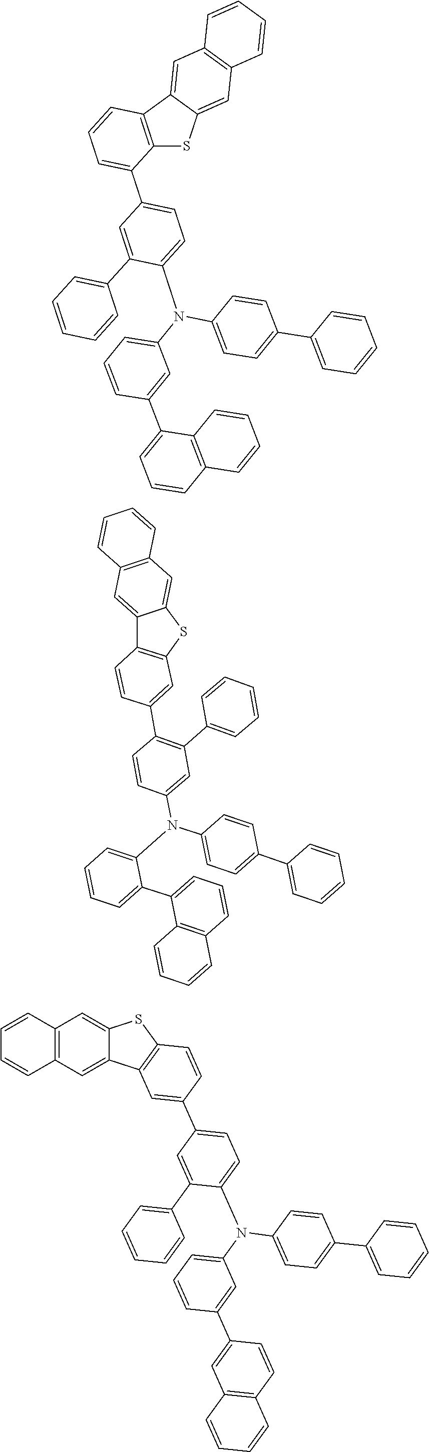

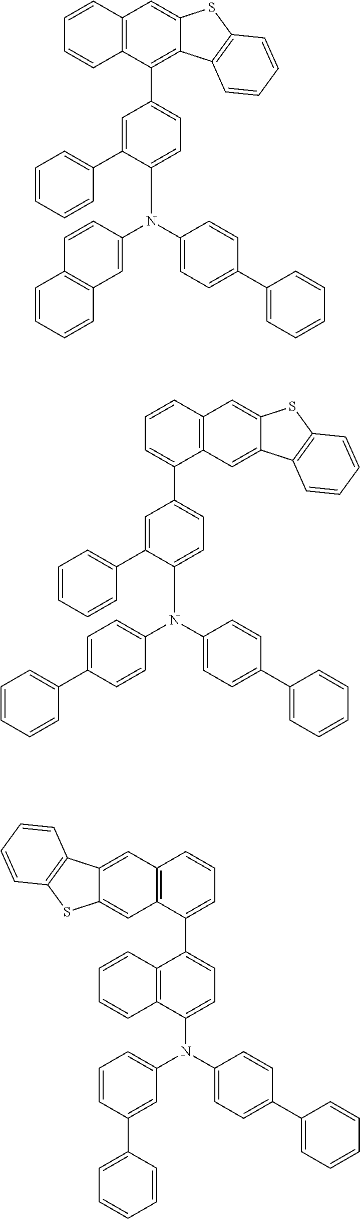 [SMILES: c1ccc(-c2ccc(N(c3ccc4ccccc4c3)c3ccc(-c4c5ccccc5cc5sc6ccccc6c45)cc3-c3ccccc3)cc2)cc1 ; c1ccc(-c2ccc(N(c3ccc(-c4ccccc4)cc3)c3ccc(-c4cccc5cc6sc7ccccc7c6cc45)cc3-c3ccccc3)cc2)cc1 ; c1ccc(-c2ccc(N(c3cccc(-c4ccccc4)c3)c3ccc(-c4cccc5cc6c(cc45)sc4ccccc46)c4ccccc34)cc2)cc1]

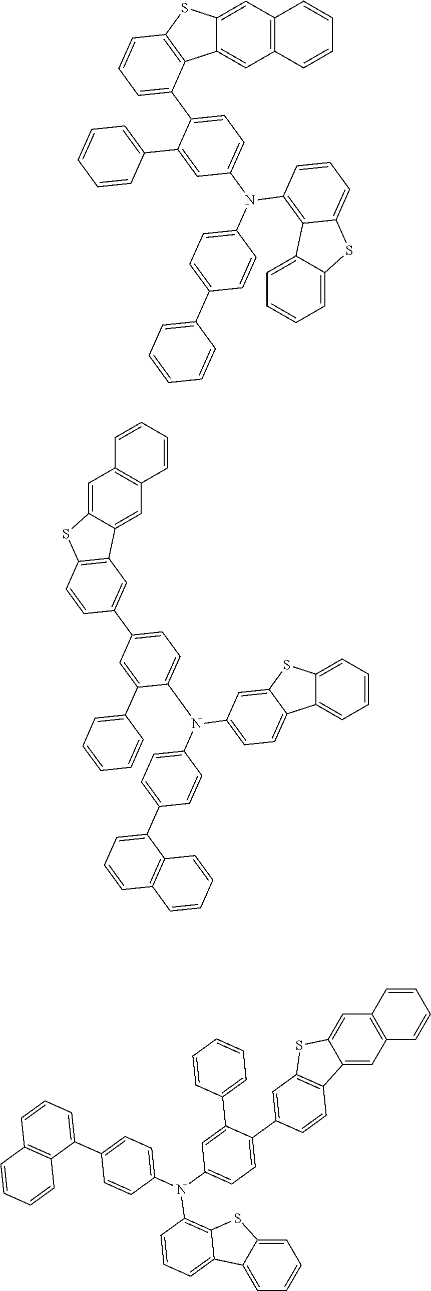 [SMILES: c1ccc(-c2ccc(N(c3ccc(-c4cccc5sc6cc7ccccc7cc6c45)c(-c4ccccc4)c3)c3cccc4sc5ccccc5c34)cc2)cc1 ; c1ccc(-c2cc(-c3ccc4sc5cc6ccccc6cc5c4c3)ccc2N(c2ccc(-c3cccc4ccccc34)cc2)c2ccc3c(c2)sc2ccccc23)cc1 ; c1ccc(-c2cc(N(c3ccc(-c4cccc5ccccc45)cc3)c3cccc4c3sc3ccccc34)ccc2-c2ccc3c(c2)sc2cc4ccccc4cc23)cc1]

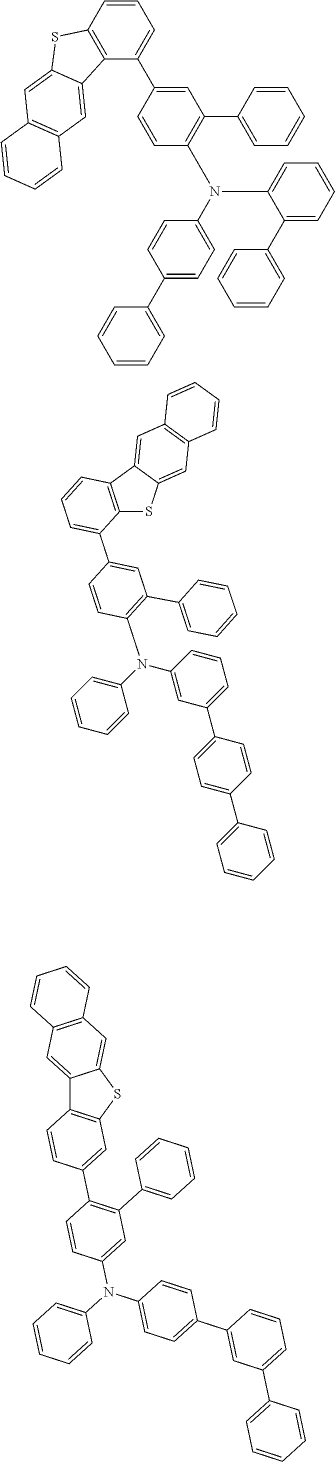

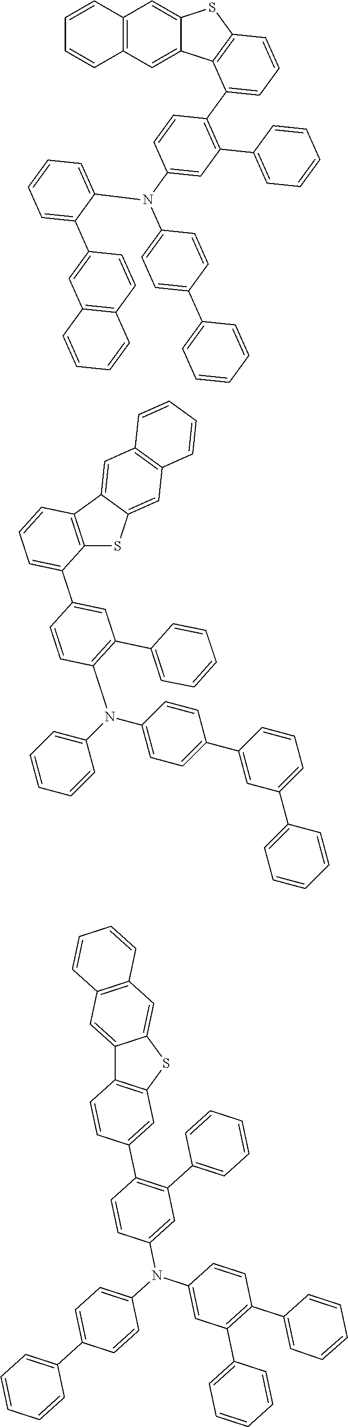

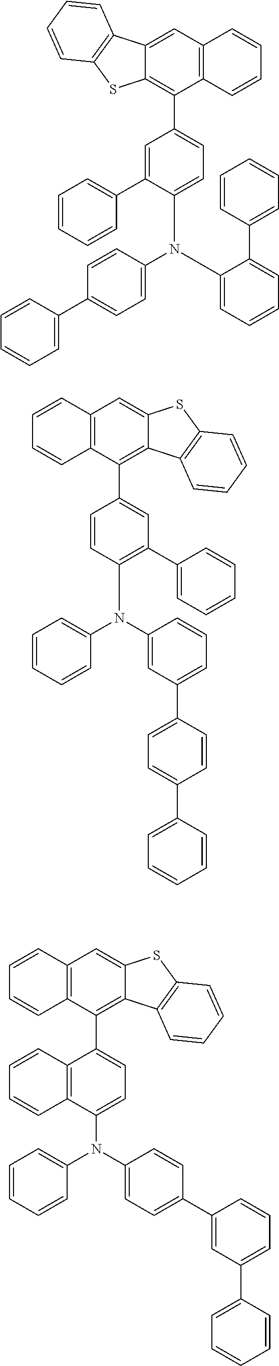

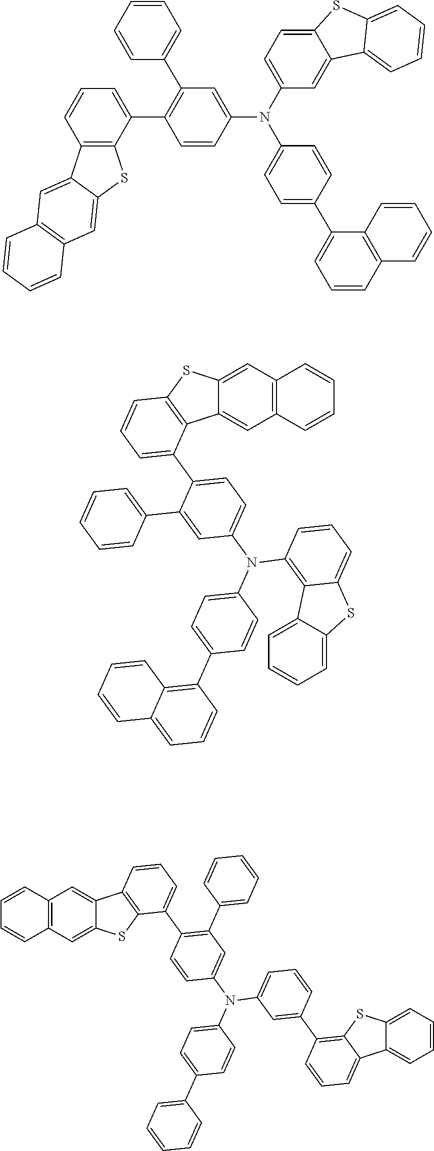 [SMILES: c1ccc(-c2cc(N(c3ccc(-c4cccc5ccccc45)cc3)c3ccc4sc5ccccc5c4c3)ccc2-c2cccc3c2sc2cc4ccccc4cc23)cc1 ; c1ccc(-c2cc(N(c3ccc(-c4cccc5ccccc45)cc3)c3cccc4sc5ccccc5c34)ccc2-c2cccc3sc4cc5ccccc5cc4c23)cc1 ; c1ccc(-c2ccc(N(c3cccc(-c4cccc5c4sc4ccccc45)c3)c3ccc(-c4cccc5c4sc4cc6ccccc6cc45)c(-c4ccccc4)c3)cc2)cc1]

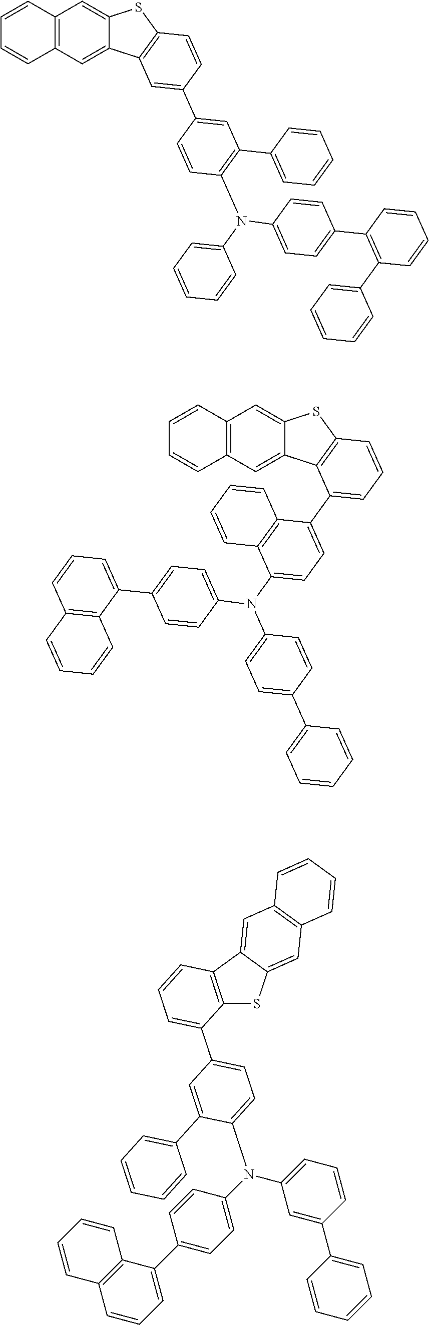 [SMILES: c1ccc(-c2ccccc2-c2ccc(N(c3ccccc3)c3ccc(-c4ccc5sc6cc7ccccc7cc6c5c4)cc3-c3ccccc3)cc2)cc1 ; c1ccc(-c2ccc(N(c3ccc(-c4cccc5ccccc45)cc3)c3ccc(-c4cccc5sc6cc7ccccc7cc6c45)c4ccccc34)cc2)cc1 ; c1ccc(-c2cccc(N(c3ccc(-c4cccc5ccccc45)cc3)c3ccc(-c4cccc5c4sc4cc6ccccc6cc45)cc3-c3ccccc3)c2)cc1]

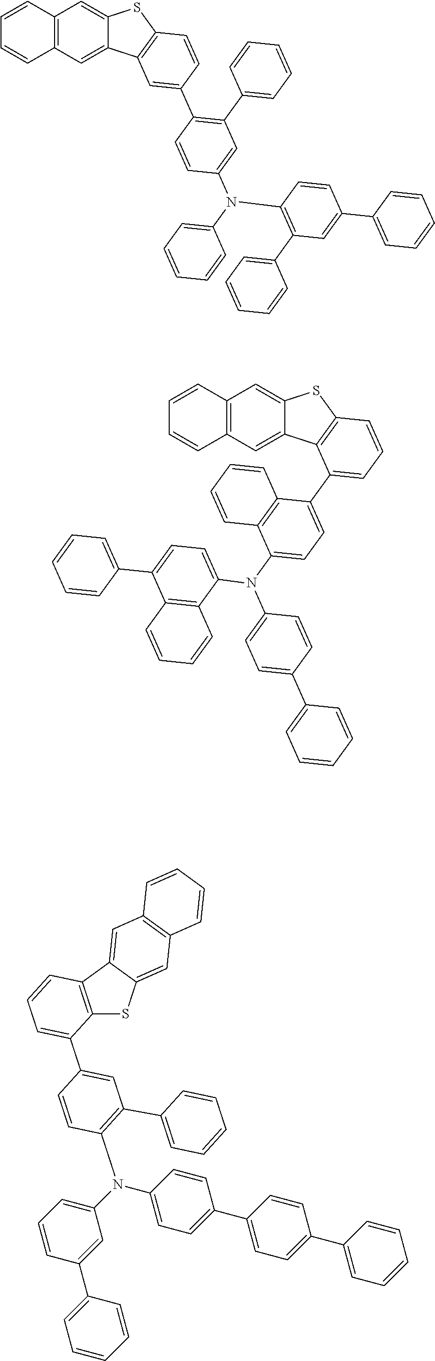 [SMILES: c1ccc(-c2ccc(N(c3ccccc3)c3ccc(-c4ccc5sc6cc7ccccc7cc6c5c4)c(-c4ccccc4)c3)c(-c3ccccc3)c2)cc1 ; c1ccc(-c2ccc(N(c3ccc(-c4ccccc4)c4ccccc34)c3ccc(-c4cccc5sc6cc7ccccc7cc6c45)c4ccccc34)cc2)cc1 ; c1ccc(-c2ccc(-c3ccc(N(c4cccc(-c5ccccc5)c4)c4ccc(-c5cccc6c5sc5cc7ccccc7cc56)cc4-c4ccccc4)cc3)cc2)cc1]

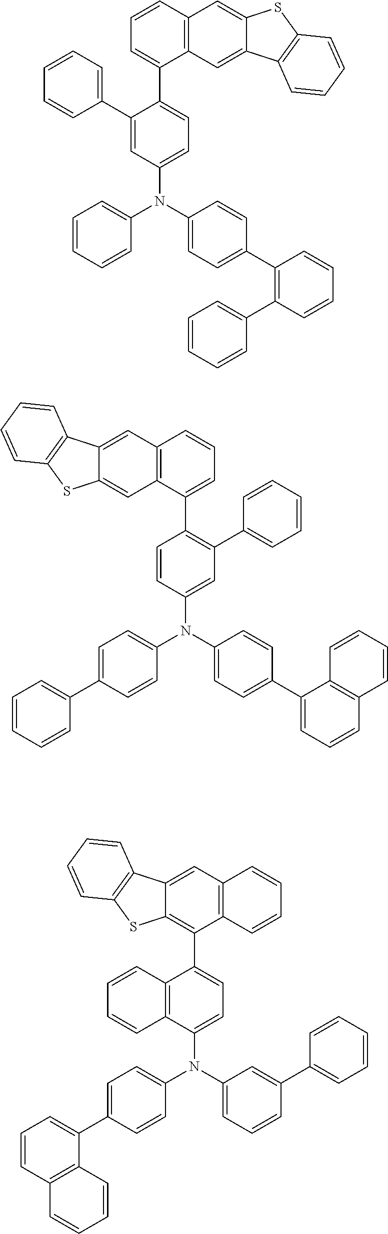 [SMILES: c1ccc(-c2ccccc2-c2ccc(N(c3ccccc3)c3ccc(-c4cccc5cc6sc7ccccc7c6cc45)c(-c4ccccc4)c3)cc2)cc1 ; c1ccc(-c2ccc(N(c3ccc(-c4cccc5ccccc45)cc3)c3ccc(-c4cccc5cc6c(cc45)sc4ccccc46)c(-c4ccccc4)c3)cc2)cc1 ; c1ccc(-c2cccc(N(c3ccc(-c4cccc5ccccc45)cc3)c3ccc(-c4c5ccccc5cc5c4sc4ccccc45)c4ccccc34)c2)cc1]

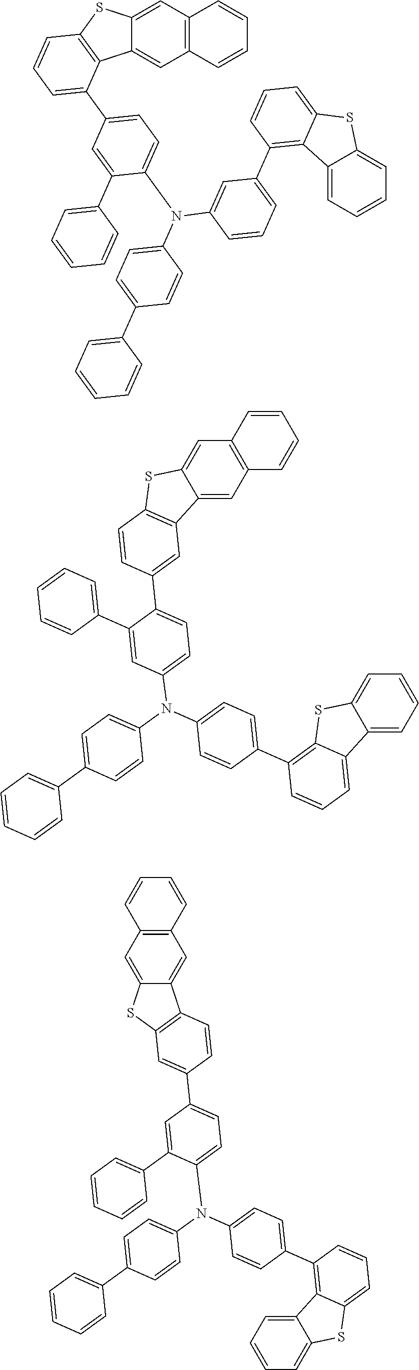 [SMILES: c1ccc(-c2ccc(N(c3cccc(-c4cccc5sc6ccccc6c45)c3)c3ccc(-c4cccc5sc6cc7ccccc7cc6c45)cc3-c3ccccc3)cc2)cc1 ; c1ccc(-c2ccc(N(c3ccc(-c4cccc5c4sc4ccccc45)cc3)c3ccc(-c4ccc5sc6cc7ccccc7cc6c5c4)c(-c4ccccc4)c3)cc2)cc1 ; c1ccc(-c2ccc(N(c3ccc(-c4cccc5sc6ccccc6c45)cc3)c3ccc(-c4ccc5c(c4)sc4cc6ccccc6cc45)cc3-c3ccccc3)cc2)cc1]

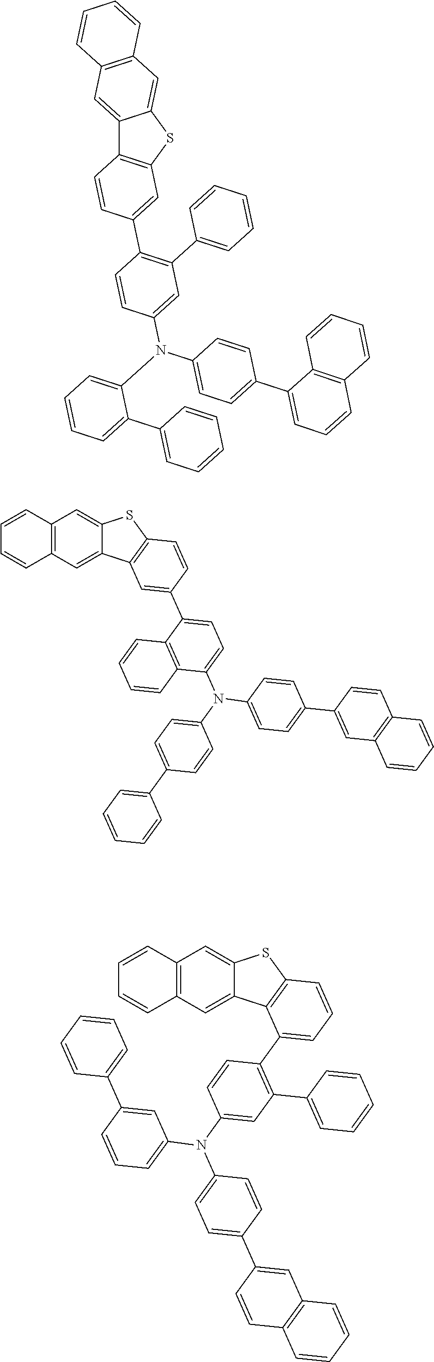 [SMILES: c1ccc(-c2cc(N(c3ccc(-c4cccc5ccccc45)cc3)c3ccccc3-c3ccccc3)ccc2-c2ccc3c(c2)sc2cc4ccccc4cc23)cc1 ; c1ccc(-c2ccc(N(c3ccc(-c4ccc5ccccc5c4)cc3)c3ccc(-c4ccc5sc6cc7ccccc7cc6c5c4)c4ccccc34)cc2)cc1 ; c1ccc(-c2cccc(N(c3ccc(-c4ccc5ccccc5c4)cc3)c3ccc(-c4cccc5sc6cc7ccccc7cc6c45)c(-c4ccccc4)c3)c2)cc1]

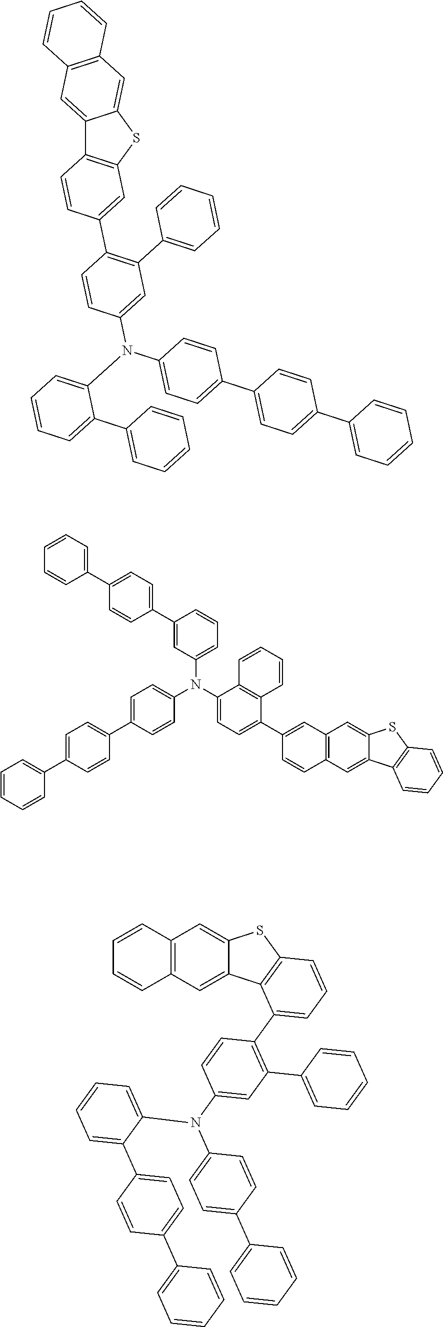 [SMILES: c1ccc(-c2ccc(-c3ccc(N(c4ccc(-c5ccc6c(c5)sc5cc7ccccc7cc56)c(-c5ccccc5)c4)c4ccccc4-c4ccccc4)cc3)cc2)cc1 ; c1ccc(-c2ccc(-c3ccc(N(c4cccc(-c5ccc(-c6ccccc6)cc5)c4)c4ccc(-c5ccc6cc7c(cc6c5)sc5ccccc57)c5ccccc45)cc3)cc2)cc1 ; c1ccc(-c2ccc(-c3ccccc3N(c3ccc(-c4ccccc4)cc3)c3ccc(-c4cccc5sc6cc7ccccc7cc6c45)c(-c4ccccc4)c3)cc2)cc1]

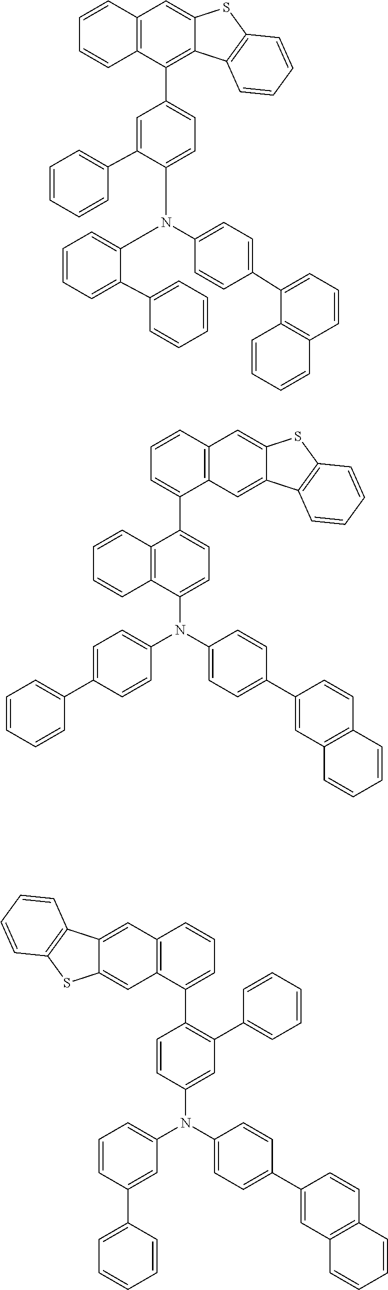 [SMILES: c1ccc(-c2ccccc2N(c2ccc(-c3cccc4ccccc34)cc2)c2ccc(-c3c4ccccc4cc4sc5ccccc5c34)cc2-c2ccccc2)cc1 ; c1ccc(-c2ccc(N(c3ccc(-c4ccc5ccccc5c4)cc3)c3ccc(-c4cccc5cc6sc7ccccc7c6cc45)c4ccccc34)cc2)cc1 ; c1ccc(-c2cccc(N(c3ccc(-c4ccc5ccccc5c4)cc3)c3ccc(-c4cccc5cc6c(cc45)sc4ccccc46)c(-c4ccccc4)c3)c2)cc1]

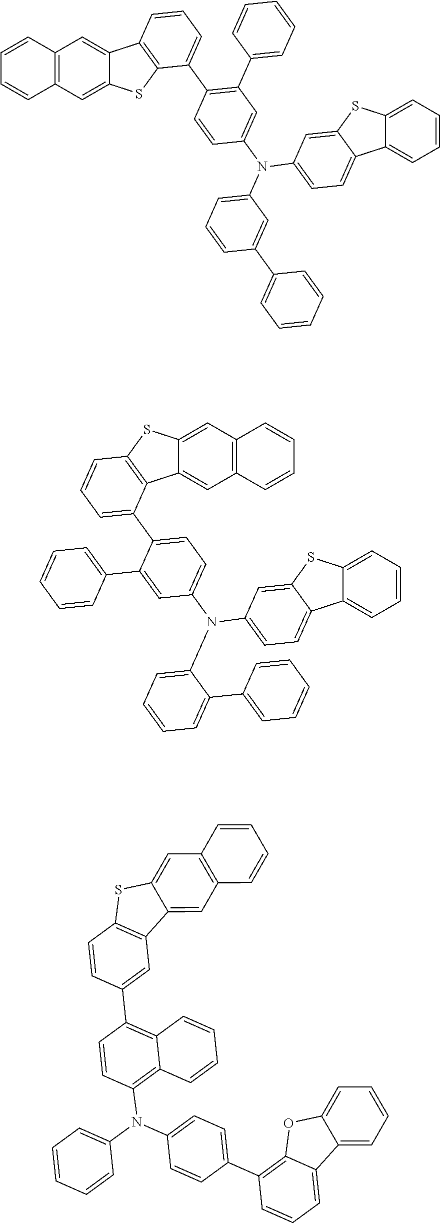 [SMILES: c1ccc(-c2cccc(N(c3ccc(-c4cccc5c4sc4cc6ccccc6cc45)c(-c4ccccc4)c3)c3ccc4c(c3)sc3ccccc34)c2)cc1 ; c1ccc(-c2cc(N(c3ccc4c(c3)sc3ccccc34)c3ccccc3-c3ccccc3)ccc2-c2cccc3sc4cc5ccccc5cc4c23)cc1 ; c1ccc(N(c2ccc(-c3cccc4c3oc3ccccc34)cc2)c2ccc(-c3ccc4sc5cc6ccccc6cc5c4c3)c3ccccc23)cc1]

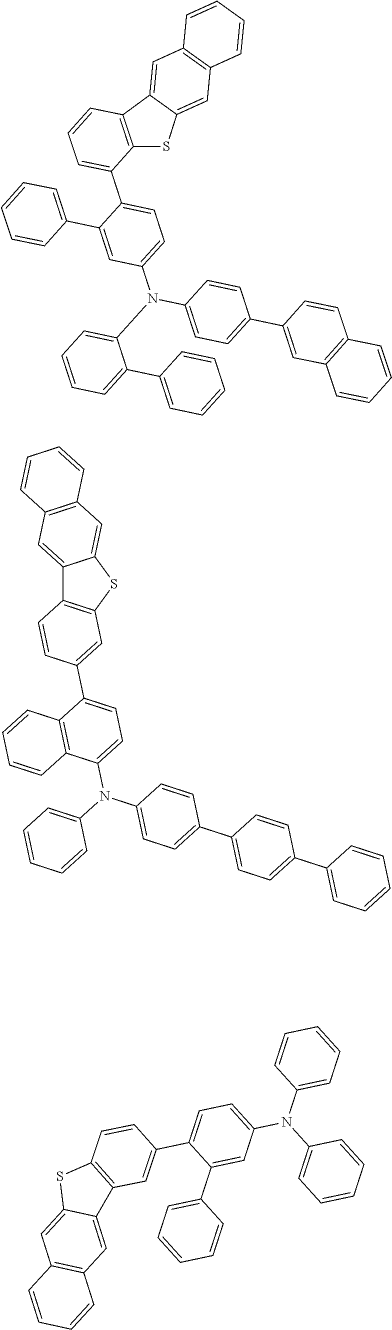 [SMILES: c1ccc(-c2cc(N(c3ccc(-c4ccc5ccccc5c4)cc3)c3ccccc3-c3ccccc3)ccc2-c2cccc3c2sc2cc4ccccc4cc23)cc1 ; c1ccc(-c2ccc(-c3ccc(N(c4ccccc4)c4ccc(-c5ccc6c(c5)sc5cc7ccccc7cc56)c5ccccc45)cc3)cc2)cc1 ; c1ccc(-c2cc(N(c3ccccc3)c3ccccc3)ccc2-c2ccc3sc4cc5ccccc5cc4c3c2)cc1]

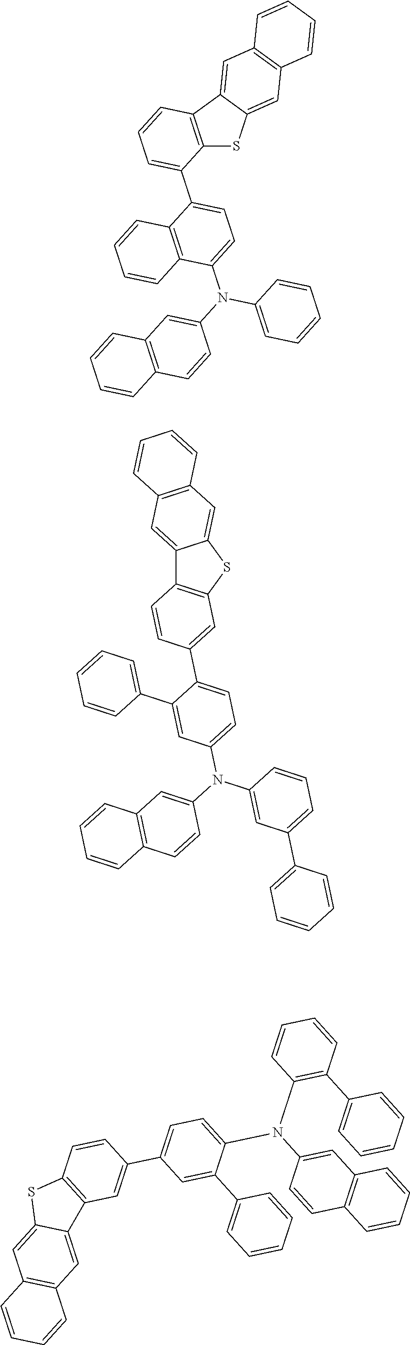

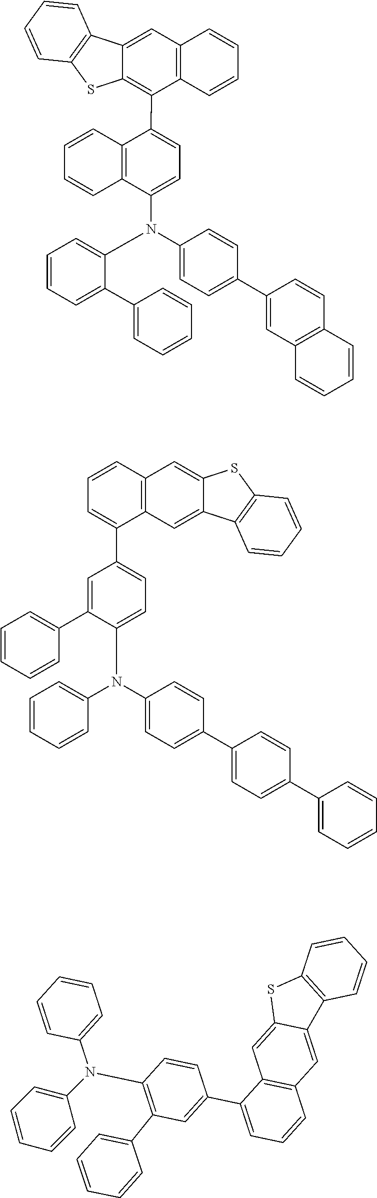 [SMILES: c1ccc(-c2ccccc2N(c2ccc(-c3ccc4ccccc4c3)cc2)c2ccc(-c3c4ccccc4cc4c3sc3ccccc34)c3ccccc23)cc1 ; c1ccc(-c2ccc(-c3ccc(N(c4ccccc4)c4ccc(-c5cccc6cc7sc8ccccc8c7cc56)cc4-c4ccccc4)cc3)cc2)cc1 ; c1ccc(-c2cc(-c3cccc4cc5c(cc34)sc3ccccc35)ccc2N(c2ccccc2)c2ccccc2)cc1]

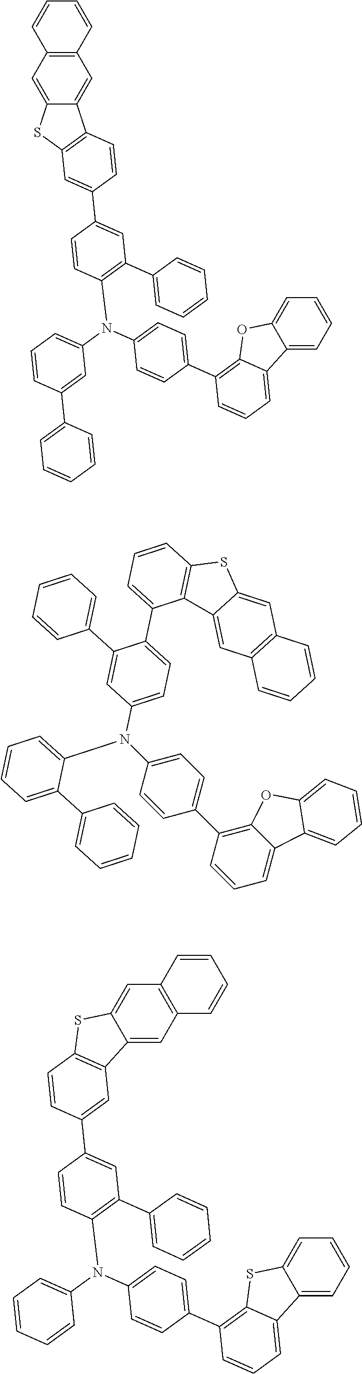 [SMILES: c1ccc(-c2cccc(N(c3ccc(-c4cccc5c4oc4ccccc45)cc3)c3ccc(-c4ccc5c(c4)sc4cc6ccccc6cc45)cc3-c3ccccc3)c2)cc1 ; c1ccc(-c2cc(N(c3ccc(-c4cccc5c4oc4ccccc45)cc3)c3ccccc3-c3ccccc3)ccc2-c2cccc3sc4cc5ccccc5cc4c23)cc1 ; c1ccc(-c2cc(-c3ccc4sc5cc6ccccc6cc5c4c3)ccc2N(c2ccccc2)c2ccc(-c3cccc4c3sc3ccccc34)cc2)cc1]

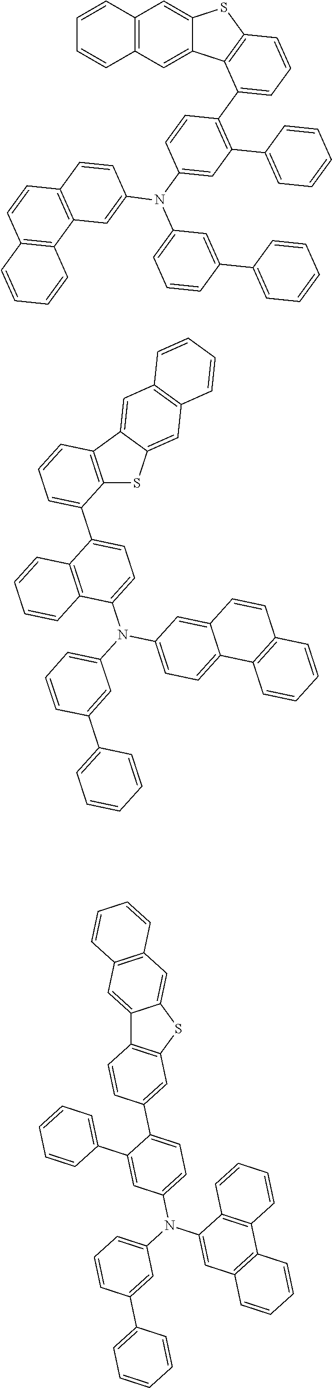 [SMILES: c1ccc(-c2cccc(N(c3ccc(-c4cccc5sc6cc7ccccc7cc6c45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4cccc5c4sc4cc6ccccc6cc45)c4ccccc34)c2)cc1 ; c1ccc(-c2cccc(N(c3ccc(-c4ccc5c(c4)sc4cc6ccccc6cc45)c(-c4ccccc4)c3)c3cc4ccccc4c4ccccc34)c2)cc1]

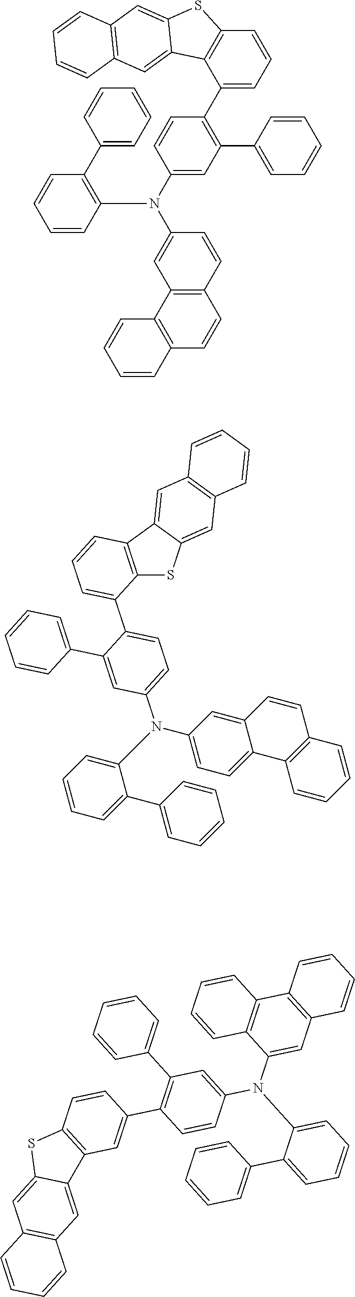 [SMILES: c1ccc(-c2cc(N(c3ccc4ccc5ccccc5c4c3)c3ccccc3-c3ccccc3)ccc2-c2cccc3sc4cc5ccccc5cc4c23)cc1 ; c1ccc(-c2cc(N(c3ccc4c(ccc5ccccc54)c3)c3ccccc3-c3ccccc3)ccc2-c2cccc3c2sc2cc4ccccc4cc23)cc1 ; c1ccc(-c2cc(N(c3ccccc3-c3ccccc3)c3cc4ccccc4c4ccccc34)ccc2-c2ccc3sc4cc5ccccc5cc4c3c2)cc1]

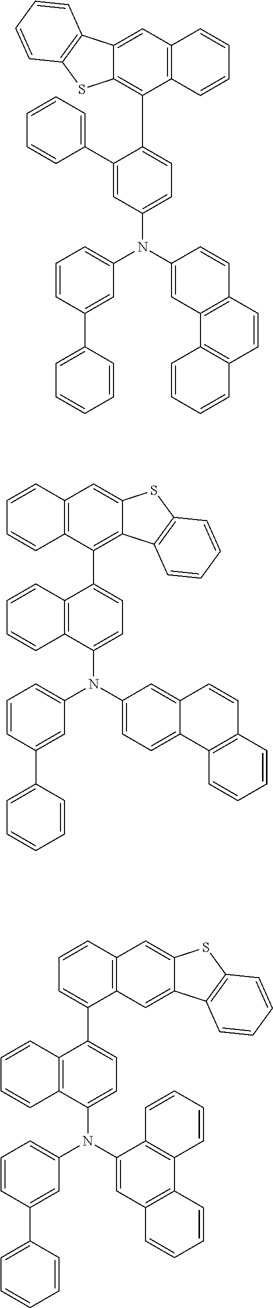 [SMILES: c1ccc(-c2cccc(N(c3ccc(-c4c5ccccc5cc5c4sc4ccccc45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4c5ccccc5cc5sc6ccccc6c45)c4ccccc34)c2)cc1 ; c1ccc(-c2cccc(N(c3ccc(-c4cccc5cc6sc7ccccc7c6cc45)c4ccccc34)c3cc4ccccc4c4ccccc34)c2)cc1]

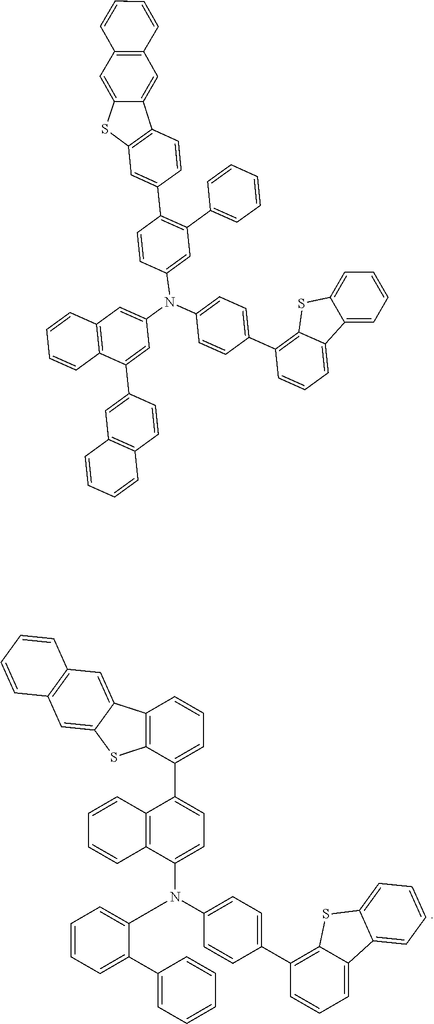 [SMILES: c1ccc(-c2cc(N(c3ccc(-c4cccc5c4sc4ccccc45)cc3)c3cc(-c4ccc5ccccc5c4)c4ccccc4c3)ccc2-c2ccc3c(c2)sc2cc4ccccc4cc23)cc1 ; c1ccc(-c2ccccc2N(c2ccc(-c3cccc4c3sc3ccccc34)cc2)c2ccc(-c3cccc4c3sc3cc5ccccc5cc34)c3ccccc23)cc1]

The compound represented by Chemical Formula 2, wherein R₇ is

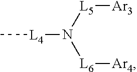

can be prepared by a preparation method as shown in the following Reaction Scheme 2 as an example, and the other remaining compounds cam be prepared in a similar manner.

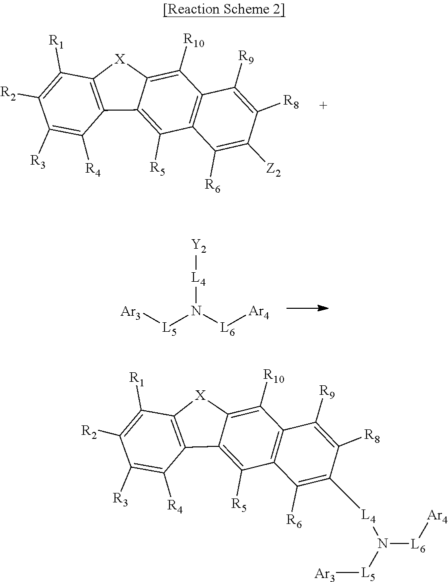

in Reaction Scheme 2, X, R₁ to R₆, R₈ to R₁₀, Ar₃, Ar₄ and L₄ to L₆ are the same as defined in Chemical Formula 2. Further, Y₂ is a boron-containing organic group, preferably a boronic acid group, a boronic acid ester group, or a boronic acid pinacol ester group, Z₂ is halogen, preferably Z₂ is chloro or bromo.

Reaction Scheme 2 is a Suzuki coupling reaction, which is preferably carried out in the presence of a palladium catalyst and a base, and a reactive group for the Suzuki coupling reaction can be modified as known in the art. The above preparation method may be further embodied in Preparation Examples described hereinafter.

Preferably, in the light emitting layer, the weight ratio of the compound represented by Chemical Formula 1 and the compound represented by Chemical Formula 2 is 10:90 to 90:10, more preferably 20:80 to 80:20, 30:70 to 70:30 or 40:60 to 60:40.

Meanwhile, the light emitting layer may further include a dopant in addition to the host. The dopant material is not particularly limited as long as it is a material used for the organic light emitting device. As an example, an aromatic amine derivative, a styrylamine compound, a boron complex, a fluoranthene compound, a metal complex, and the like can be mentioned. Specific examples of the aromatic amine derivatives include substituted or unsubstituted fused aromatic ring derivatives having an arylamino group, examples thereof include pyrene, anthracene, chrysene, and periflanthene having the arylamino group, and the like. The styrylamine compound is a compound of an arylamine, which is unsubstituted or substituted with one or two or more substituent groups selected from the group consisting of an aryl group, a silyl group, an alkyl group, a cycloalkyl group, and an arylamino group, is substituted with at least one arylvinyl group. Specific examples thereof include styrylamine, styryldiamine, styryltriamine, styryltetramine, and the like, but are not limited thereto. Further, examples of the metal complex include an iridium complex, a platinum complex, and the like, but are not limited thereto.

In one example, the dopant material may be one or more selected from the group consisting of the following, without being limited thereto:

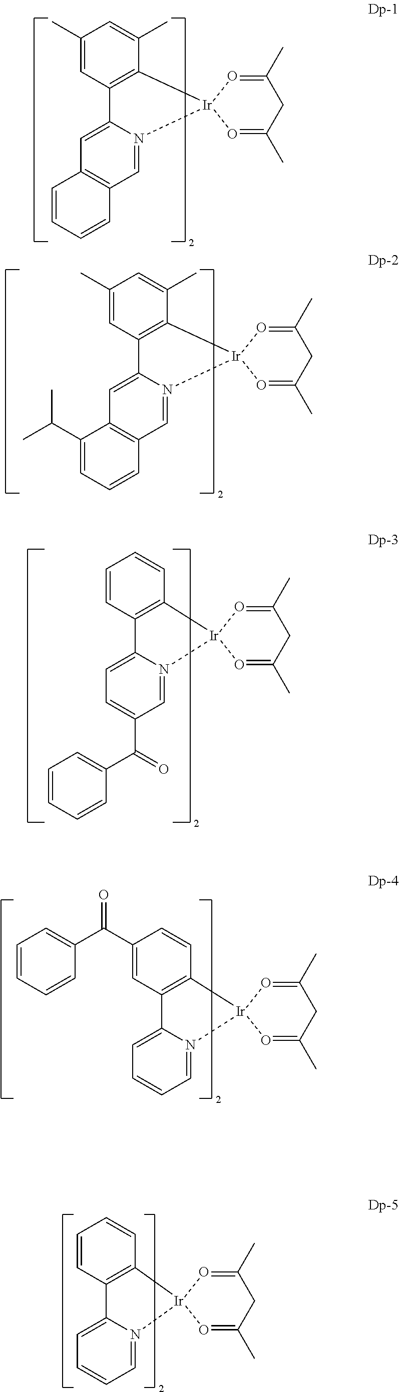

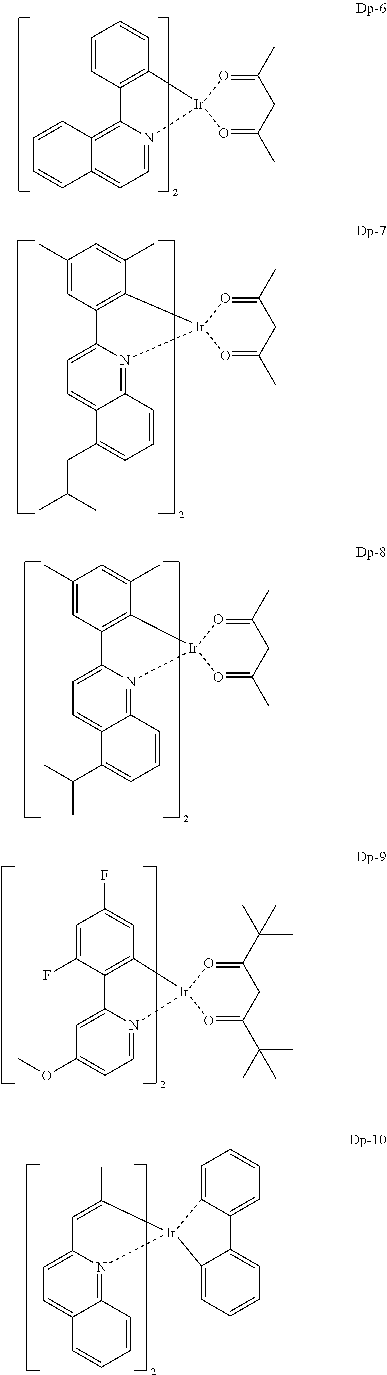

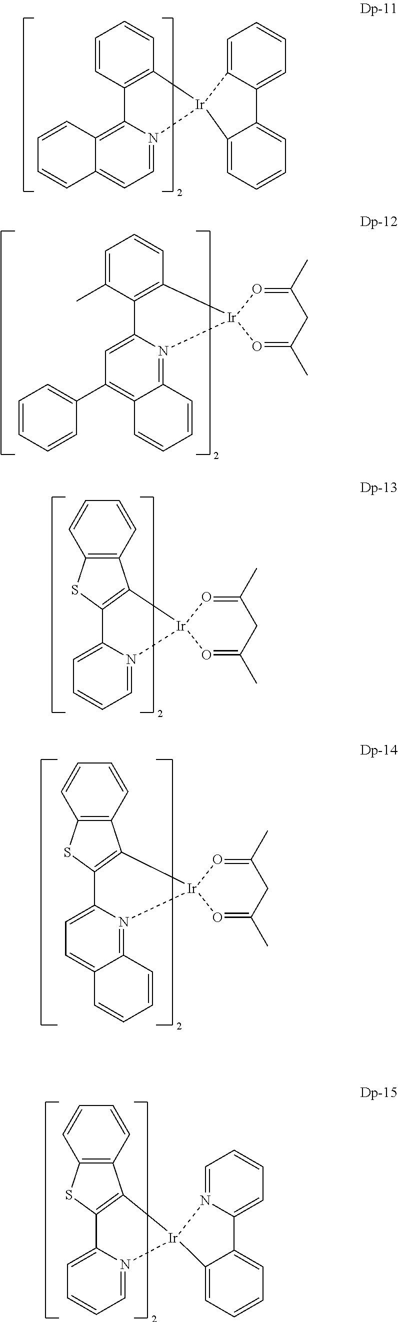

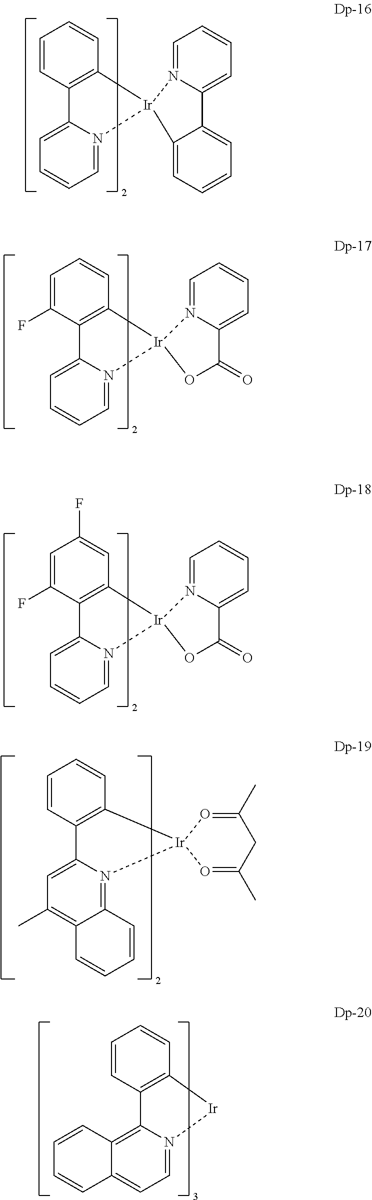

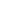

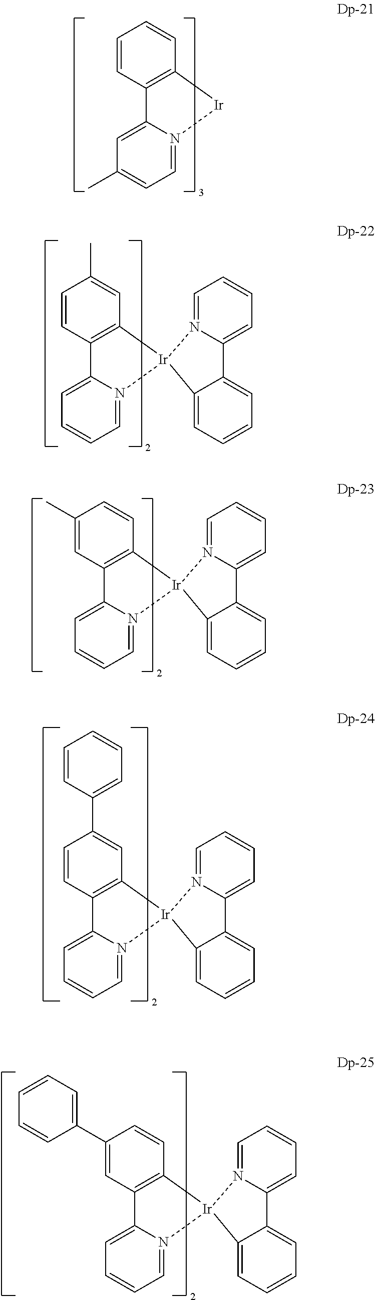

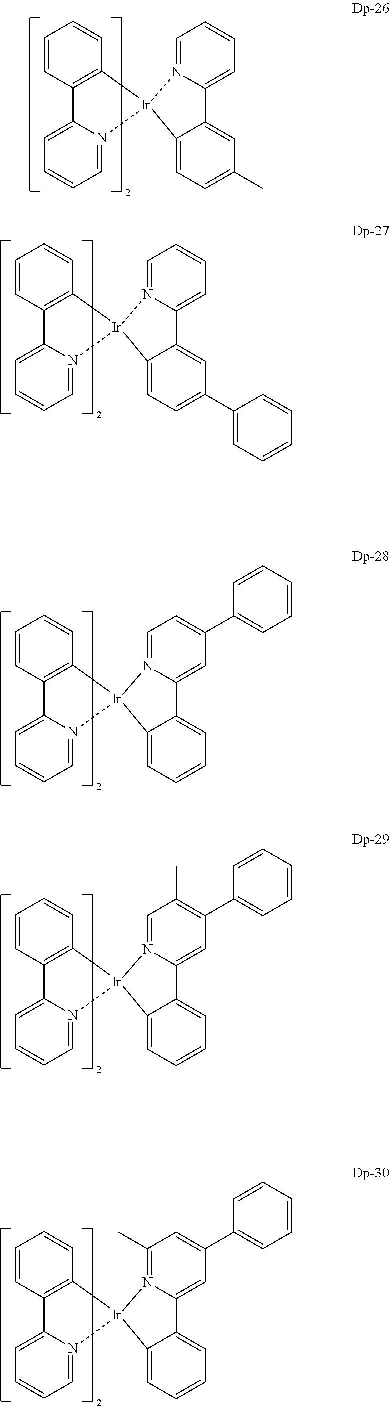

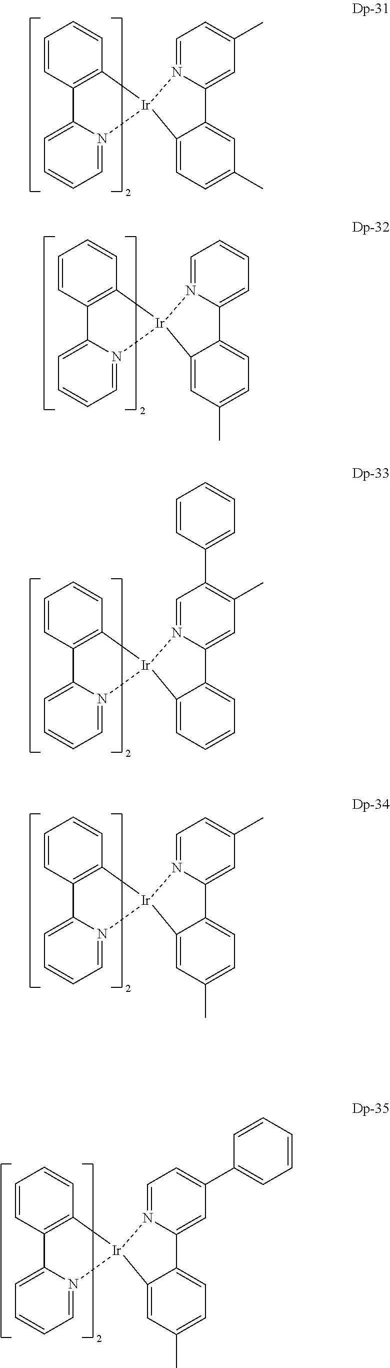

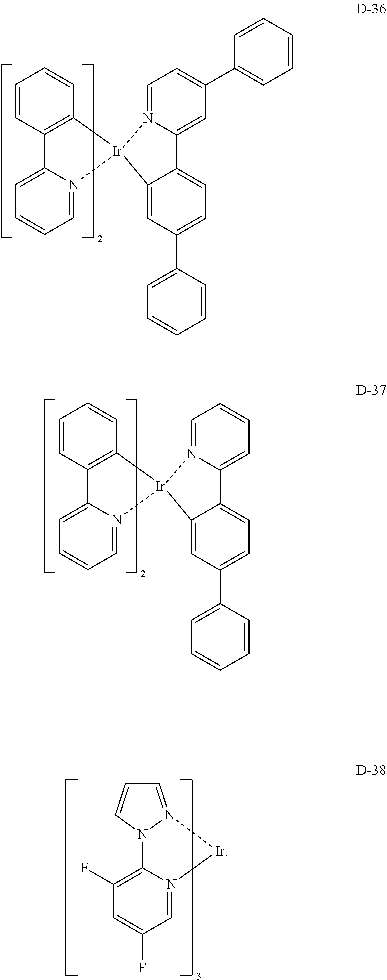

### Hole Blocking Layer

The hole blocking layer is a layer provided between the electron transport layer and the light emitting layer in order to prevent the holes injected from the anode from being transferred to the electron transport layer without being recombined in the light emitting layer, which may also be referred to as a hole inhibition layer. The hole blocking layer is preferably a material having high ionization energy.

### Electron Transport Layer

The organic light emitting device according to the present disclosure may include an electron transport layer on the light emitting layer, if necessary.

The electron transport layer is a layer that receives the electrons from the electron injection layer formed on the cathode or the cathode and transports the electrons to the light emitting layer, and that suppress the transfer of holes from the light emitting layer, and an electron transport material is suitably a material which may receive electrons well from a cathode and transfer the electrons to a light emitting layer, and has a large mobility for electrons.

Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

### Electron Injection Layer

The organic light emitting device according to the present disclosure may further include an electron injection layer on the light emitting layer (or on the electron transport layer, if the electron transport layer exists).

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, the organic light emitting device of the present disclosure may include an electron injection and transport layer that performs both the roles of the electron injection layer and the electron transport layer that inject electrons from the electrode and transport the injected electrons to the light emitting layer, instead of the electron transport layer and the electron injection layer. As such an electron injection and transport material, the above-mentioned electron injection material or electron transport material can be used.

### Organic Light Emitting Device

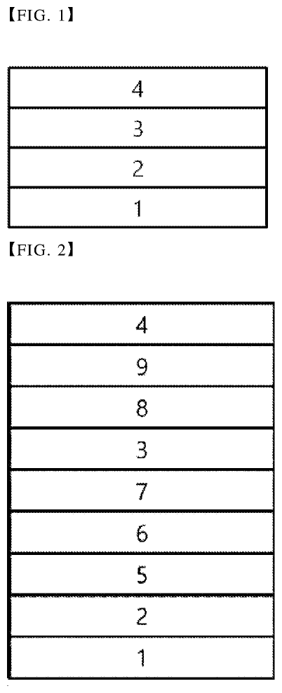

The structure of the organic light emitting device according to the present disclosure is illustrated in FIGS. 1 and 2. FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron injection and transport layer 9, and a cathode 4.

The organic light emitting device according to the present disclosure can be manufactured by sequentially stacking the above-described structures. In this case, the organic light emitting device may be manufactured by depositing a metal, metal oxides having conductivity, or an alloy thereof on the substrate by using a PVD (physical vapor deposition) method such as a sputtering method or an e-beam evaporation method to form the anode, forming the respective layers described above thereon, and then depositing a material that can be used as the cathode thereon. In addition to such a method, the organic light emitting device may be manufactured by sequentially depositing from the cathode material to the anode material on a substrate in the reverse order of the above-mentioned configuration (WO 2003/012890). Further, the light emitting layer may be formed by subjecting hosts and dopants to a vacuum deposition method and a solution coating method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure may be a bottom emission device, a top emission device, or a double-sided light emitting device, and particularly, may be a bottom emission device that requires relatively high luminous efficiency.

Hereinafter, preferred examples are presented to assist in the understanding of the present disclosure. However, the following examples are only provided for a better understanding of the present disclosure, and is not intended to limit the content of the present disclosure.

### SYNTHESIS EXAMPLE

### Preparation of Compound Represented by Chemical Formula 1

### Synthesis Example 1-1

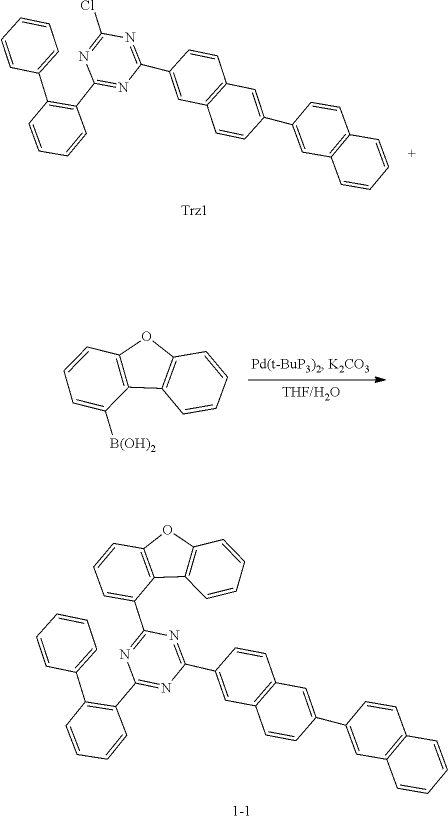 [SMILES: Clc1nc(-c2ccc3cc(-c4ccc5ccccc5c4)ccc3c2)nc(-c2ccccc2-c2ccccc2)n1 ; OB(O)c1cccc2oc3ccccc3c12 ; c1ccc(-c2ccccc2-c2nc(-c3ccc4cc(-c5ccc6ccccc6c5)ccc4c3)nc(-c3cccc4oc5ccccc5c34)n2)cc1]

Trz1 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.2 g of Compound 1-1. (Yield: 65%, MS: [M+H]+=652)

### Synthesis Example 1-2

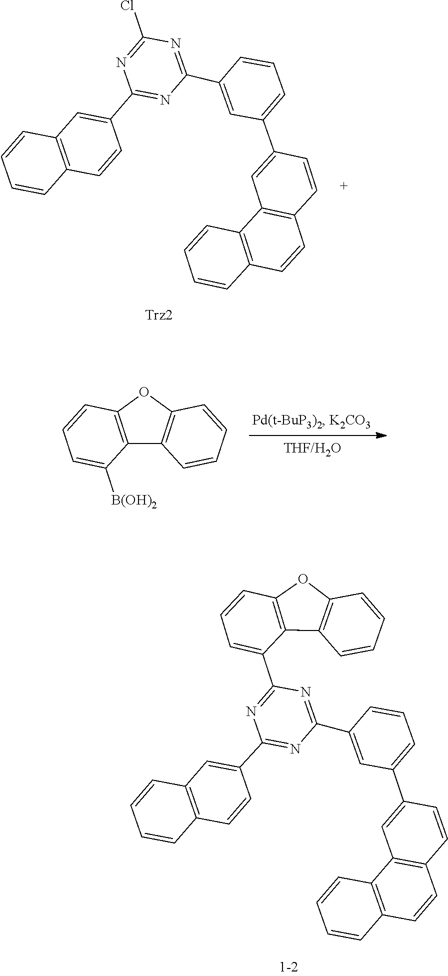 [SMILES: Clc1nc(-c2cccc(-c3ccc4ccc5ccccc5c4c3)c2)nc(-c2ccc3ccccc3c2)n1 ; OB(O)c1cccc2oc3ccccc3c12 ; c1cc(-c2ccc3ccc4ccccc4c3c2)cc(-c2nc(-c3ccc4ccccc4c3)nc(-c3cccc4oc5ccccc5c34)n2)c1]

Trz2 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14 g of Compound 1-2. (Yield: 74%, MS: [M+H]+=626)

### Synthesis Example 1-3

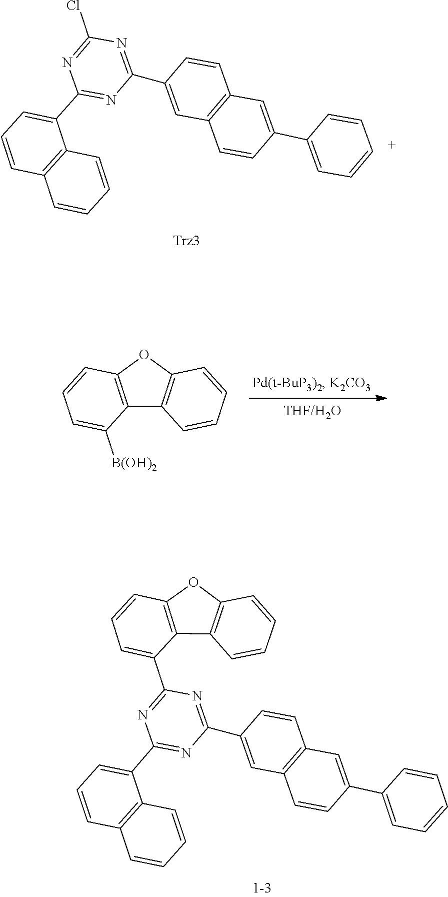 [SMILES: Clc1nc(-c2ccc3cc(-c4ccccc4)ccc3c2)nc(-c2cccc3ccccc23)n1 ; OB(O)c1cccc2oc3ccccc3c12 ; c1ccc(-c2ccc3cc(-c4nc(-c5cccc6ccccc56)nc(-c5cccc6oc7ccccc7c56)n4)ccc3c2)cc1]

Trz3 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.4 g of Compound 1-3. (Yield: 69%, MS: [M+H]+=576)

### Synthesis Example 1-4

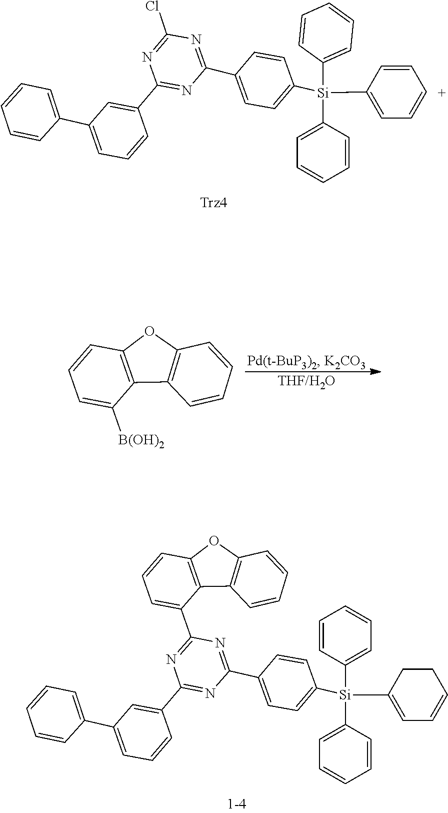 [SMILES: Clc1nc(-c2ccc([Si](C3=CC=[C+]C=C3)(c3ccccc3)c3ccccc3)cc2)nc(-c2cccc(-c3ccccc3)c2)n1 ; OB(O)c1cccc2oc3ccccc3c12 ; C1=CCCC([Si](c2ccccc2)(c2ccccc2)c2ccc(-c3nc(-c4cccc(-c5ccccc5)c4)nc(-c4cccc5oc6ccccc6c45)n3)cc2)=C1]

Trz4 (15 g, 24.9 mmol) and dibenzo[b,d]furan-1-ylboronic acid (5.5 g, 26.2 mmol) were added to 300 ml of THF, and the mixture was stirred and refluxed. Then, potassium carbonate (10.3 g, 74.7 mmol) was dissolved in 31 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-4. (Yield: 69%, MS: [M+H]+=734)

### Synthesis Example 1-5

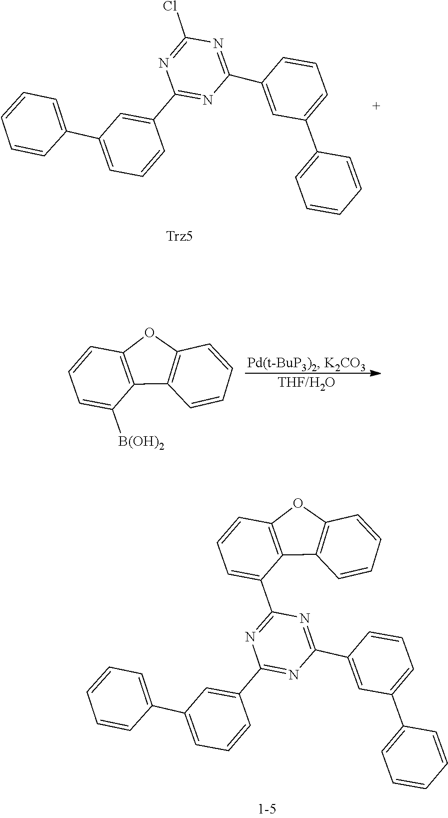 [SMILES: Clc1nc(-c2cccc(-c3ccccc3)c2)nc(-c2cccc(-c3ccccc3)c2)n1 ; OB(O)c1cccc2oc3ccccc3c12 ; c1ccc(-c2cccc(-c3nc(-c4cccc(-c5ccccc5)c4)nc(-c4cccc5oc6ccccc6c45)n3)c2)cc1]

Trz5 (15 g, 30.2 mmol) and dibenzo[b,d]furan-1-ylboronic acid (6.7 g, 31.8 mmol) were added to 300 ml of THF, and the mixture was stirred and refluxed. Then, potassium carbonate (12.5 g, 90.7 mmol) was dissolved in 38 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.5 g of Compound 1-5. (Yield: 66%, MS: [M+H]+=629)

### Synthesis Example 1-6

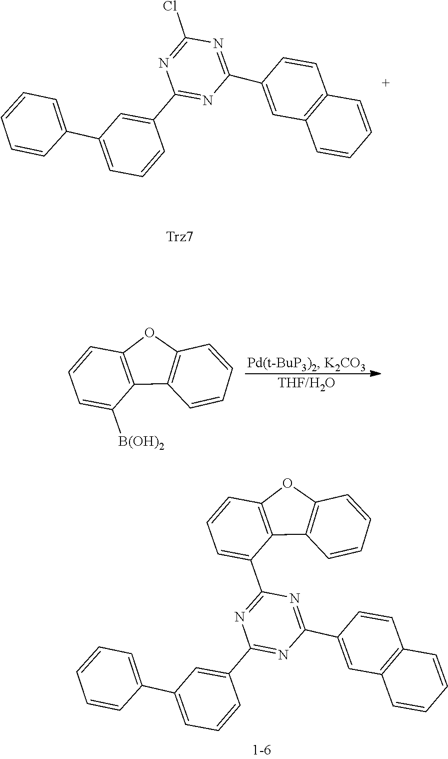 [SMILES: Clc1nc(-c2cccc(-c3ccccc3)c2)nc(-c2ccc3ccccc3c2)n1 ; OB(O)c1cccc2oc3ccccc3c12 ; c1ccc(-c2cccc(-c3nc(-c4ccc5ccccc5c4)nc(-c4cccc5oc6ccccc6c45)n3)c2)cc1]

Trz7 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.6 g of Compound 1-6. (Yield: 70%, MS: [M+H]+=576)

### Synthesis Example 1-7

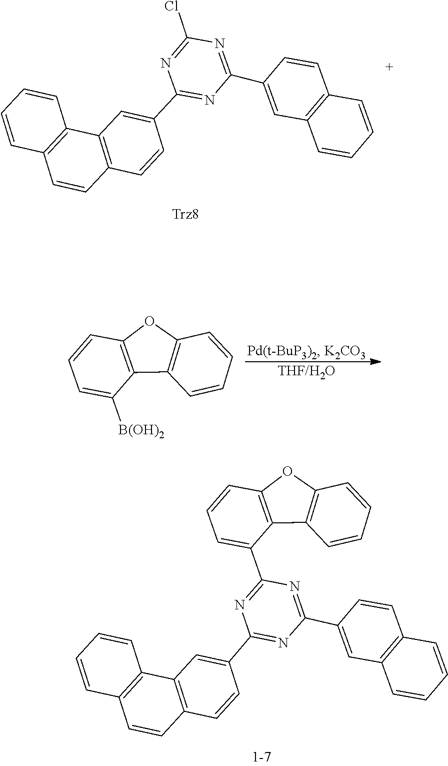 [SMILES: Clc1nc(-c2ccc3ccccc3c2)nc(-c2ccc3ccc4ccccc4c3c2)n1 ; OB(O)c1cccc2oc3ccccc3c12 ; c1ccc2cc(-c3nc(-c4ccc5ccc6ccccc6c5c4)nc(-c4cccc5oc6ccccc6c45)n3)ccc2c1]

Trz8 (15 g, 35.9 mmol) and dibenzo[b,d]furan-1-ylboronic acid (8 g, 37.7 mmol) were added to 300 ml of THF, and the mixture was stirred and refluxed. Then, potassium carbonate (14.9 g, 107.7 mmol) was dissolved in 45 ml of water and added thereto, and the mixture was sufficiently stirred and then bis(tri-tert-butylphosphine)palladium(0) (0.2 g, 0.4 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.8 g of Compound 1-7. (Yield: 70%, MS: [M+H]+=550)

### Synthesis Example 1-8

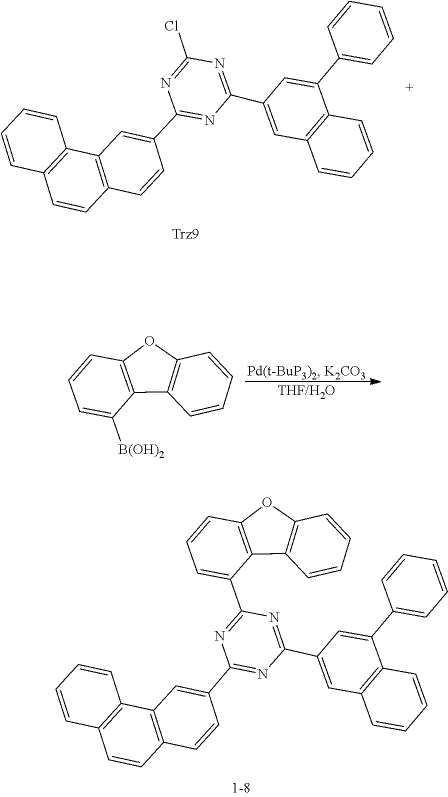 [SMILES: Clc1nc(-c2cc(-c3ccccc3)c3ccccc3c2)nc(-c2ccc3ccc4ccccc4c3c2)n1 ; OB(O)c1cccc2oc3ccccc3c12 ; c1ccc(-c2cc(-c3nc(-c4ccc5ccc6ccccc6c5c4)nc(-c4cccc5oc6ccccc6c45)n3)cc3ccccc23)cc1]

Trz9 (15 g, 30.4 mmol) and dibenzo[b,d]furan-1-ylboronic acid (6.8 g, 31.9 mmol) were added to 300 ml of THF, and the mixture was stirred and refluxed. Then, potassium carbonate (12.6 g, 91.1 mmol) was dissolved in 38 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.7 g of Compound 1-8. (Yield: 72%, MS: [M+H]+=626)

### Synthesis Example 1-9

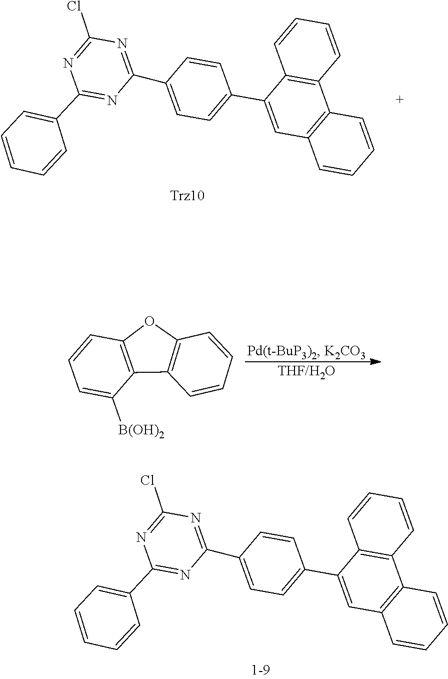 [SMILES: Clc1nc(-c2ccccc2)nc(-c2ccc(-c3cc4ccccc4c4ccccc34)cc2)n1 ; OB(O)c1cccc2oc3ccccc3c12 ; Clc1nc(-c2ccccc2)nc(-c2ccc(-c3cc4ccccc4c4ccccc34)cc2)n1]

Trz10 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-9. (Yield: 73%, MS: [M+H]+=576)

### Synthesis Example 1-10

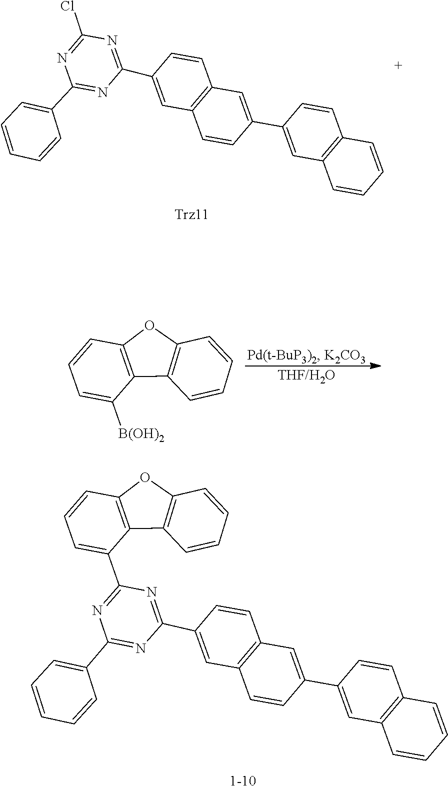 [SMILES: Clc1nc(-c2ccccc2)nc(-c2ccc3cc(-c4ccc5ccccc5c4)ccc3c2)n1 ; OB(O)c1cccc2oc3ccccc3c12 ; c1ccc(-c2nc(-c3ccc4cc(-c5ccc6ccccc6c5)ccc4c3)nc(-c3cccc4oc5ccccc5c34)n2)cc1]

Trz11 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.4 g of Compound 1-10. (Yield: 69%, MS: [M+H]+=576)

### Synthesis Example 1-11

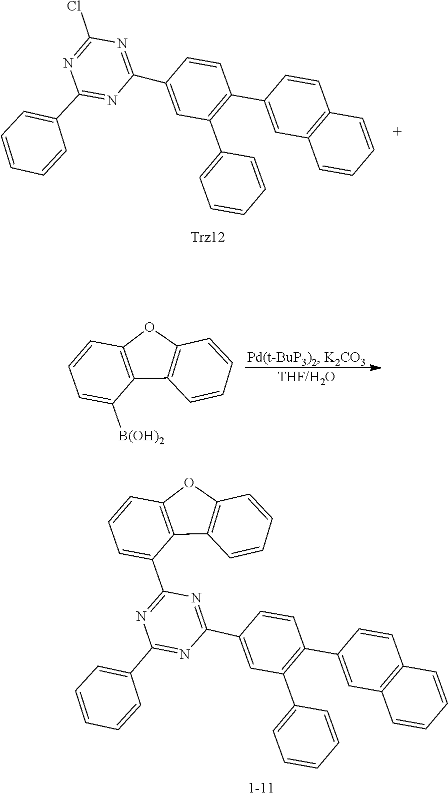 [SMILES: Clc1nc(-c2ccccc2)nc(-c2ccc(-c3ccc4ccccc4c3)c(-c3ccccc3)c2)n1 ; OB(O)c1cccc2oc3ccccc3c12 ; c1ccc(-c2nc(-c3ccc(-c4ccc5ccccc5c4)c(-c4ccccc4)c3)nc(-c3cccc4oc5ccccc5c34)n2)cc1]

Trz12 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-11. (Yield: 74%, MS: [M+H]+=602)

### Synthesis Example 1-12

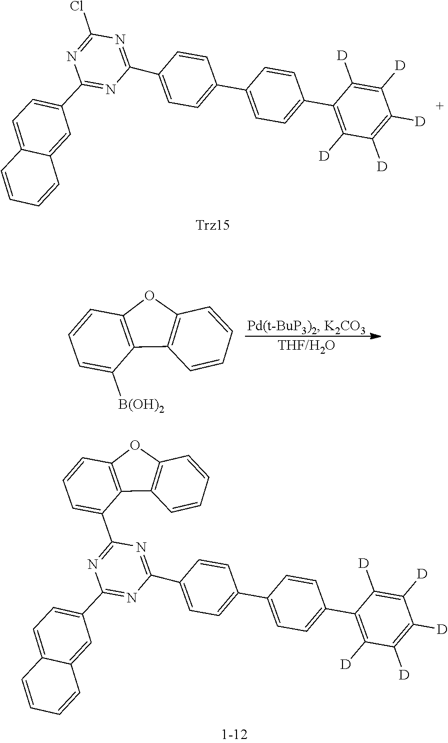 [SMILES: [2H]c1c([2H])c([2H])c(-c2ccc(-c3ccc(-c4nc(Cl)nc(-c5ccc6ccccc6c5)n4)cc3)cc2)c([2H])c1[2H] ; OB(O)c1cccc2oc3ccccc3c12 ; [2H]c1c([2H])c([2H])c(-c2ccc(-c3ccc(-c4nc(-c5ccc6ccccc6c5)nc(-c5cccc6oc7ccccc7c56)n4)cc3)cc2)c([2H])c1[2H]]

Trz15 (15 g, 31.6 mmol) and dibenzo[b,d]furan-1-ylboronic acid (7 g, 33.2 mmol) were added to 300 ml of THF, and the mixture was stirred and refluxed. Then, potassium carbonate (13.1 g, 94.7 mmol) was dissolved in 39 ml of water and added thereto, and the mixture was sufficiently stirred and then bis(tri-tert-butylphosphine)palladium(0) (0.2 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-12. (Yield: 74%, MS: [M+H]+=607)

### Synthesis Example 1-13

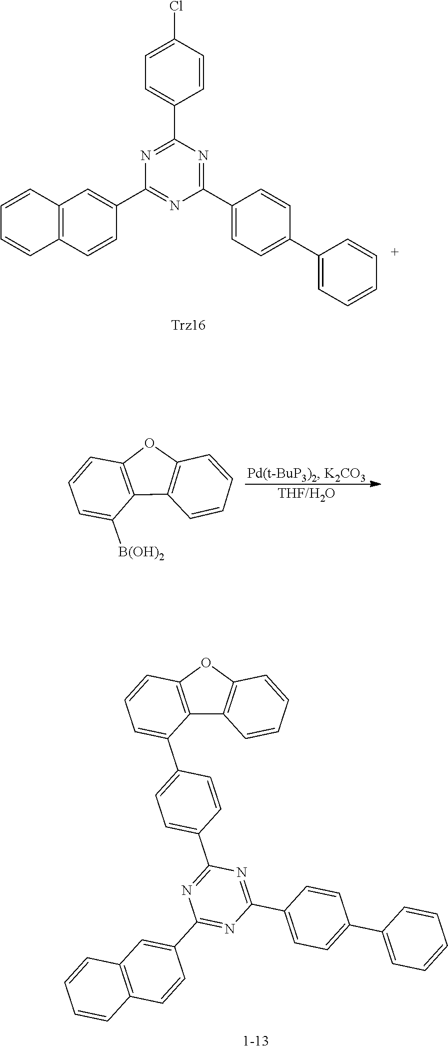 [SMILES: Clc1ccc(-c2nc(-c3ccc(-c4ccccc4)cc3)nc(-c3ccc4ccccc4c3)n2)cc1 ; OB(O)c1cccc2oc3ccccc3c12 ; c1ccc(-c2ccc(-c3nc(-c4ccc(-c5cccc6oc7ccccc7c56)cc4)nc(-c4ccc5ccccc5c4)n3)cc2)cc1]

Trz16 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.7 g of Compound 1-13. (Yield: 66%, MS: [M+H]+=602)

### Synthesis Example 1-14

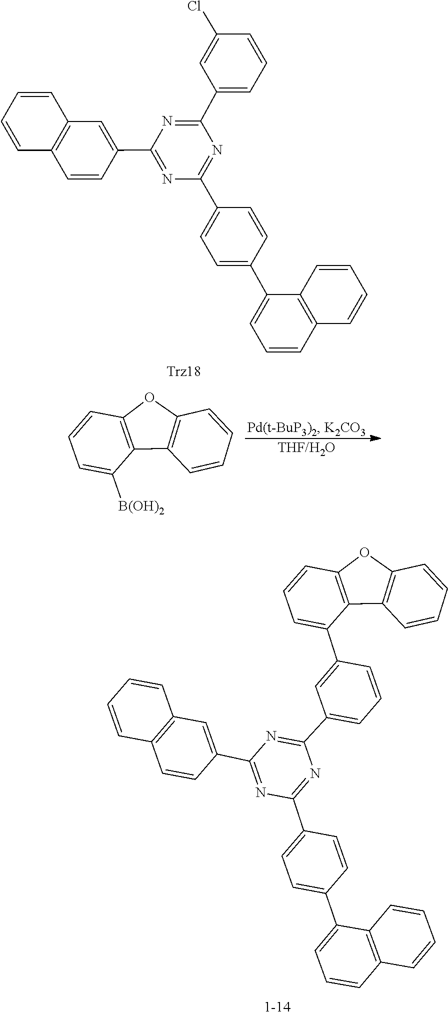 [SMILES: Clc1cccc(-c2nc(-c3ccc(-c4cccc5ccccc45)cc3)nc(-c3ccc4ccccc4c3)n2)c1 ; OB(O)c1cccc2oc3ccccc3c12 ; c1cc(-c2nc(-c3ccc(-c4cccc5ccccc45)cc3)nc(-c3ccc4ccccc4c3)n2)cc(-c2cccc3oc4ccccc4c23)c1]

Trz18 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-14. (Yield: 71%, MS: [M+H]+=652)

### Synthesis Example 1-15

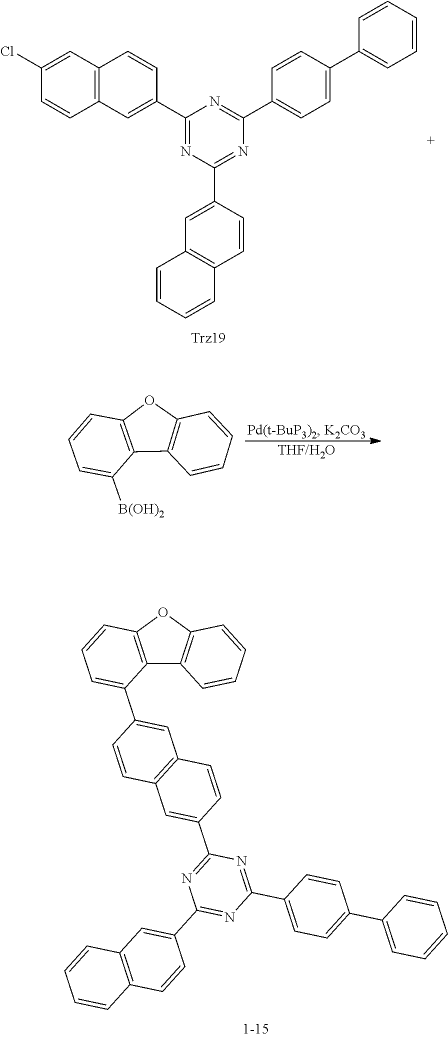 [SMILES: Clc1ccc2cc(-c3nc(-c4ccc(-c5ccccc5)cc4)nc(-c4ccc5ccccc5c4)n3)ccc2c1 ; OB(O)c1cccc2oc3ccccc3c12 ; c1ccc(-c2ccc(-c3nc(-c4ccc5ccccc5c4)nc(-c4ccc5cc(-c6cccc7oc8ccccc8c67)ccc5c4)n3)cc2)cc1]

Trz19 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.7 g of Compound 1-15. (Yield: 73%, MS: [M+H]+=652)

### Synthesis Example 1-16

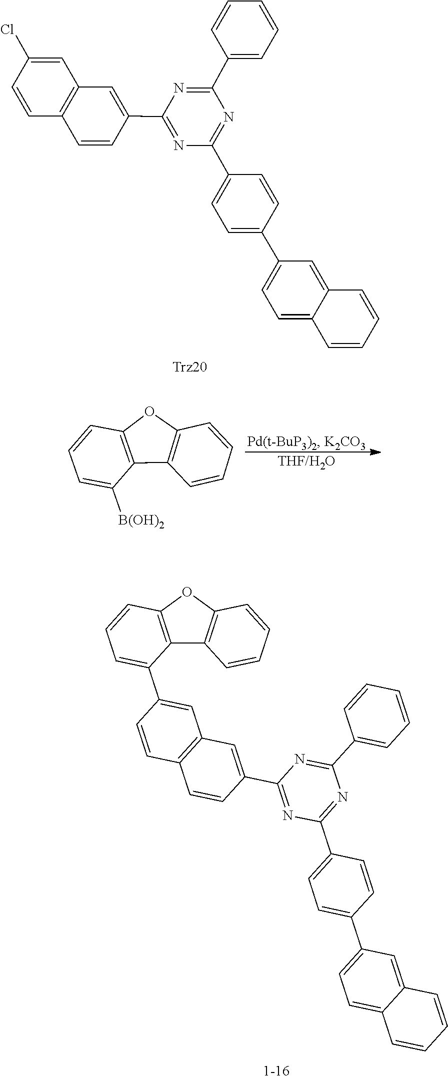 [SMILES: Clc1ccc2ccc(-c3nc(-c4ccccc4)nc(-c4ccc(-c5ccc6ccccc6c5)cc4)n3)cc2c1 ; OB(O)c1cccc2oc3ccccc3c12 ; c1ccc(-c2nc(-c3ccc(-c4ccc5ccccc5c4)cc3)nc(-c3ccc4ccc(-c5cccc6oc7ccccc7c56)cc4c3)n2)cc1]

Trz20 (15 g, 28.8 mmol) and dibenzo[b,d]furan-1-ylboronic acid (6.4 g, 30.3 mmol) were added to 300 ml of THF, and the mixture was stirred and refluxed. Then, potassium carbonate (12 g, 86.5 mmol) was dissolved in 36 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-16. (Yield: 67%, MS: [M+H]+=652)

### Synthesis Example 1-17

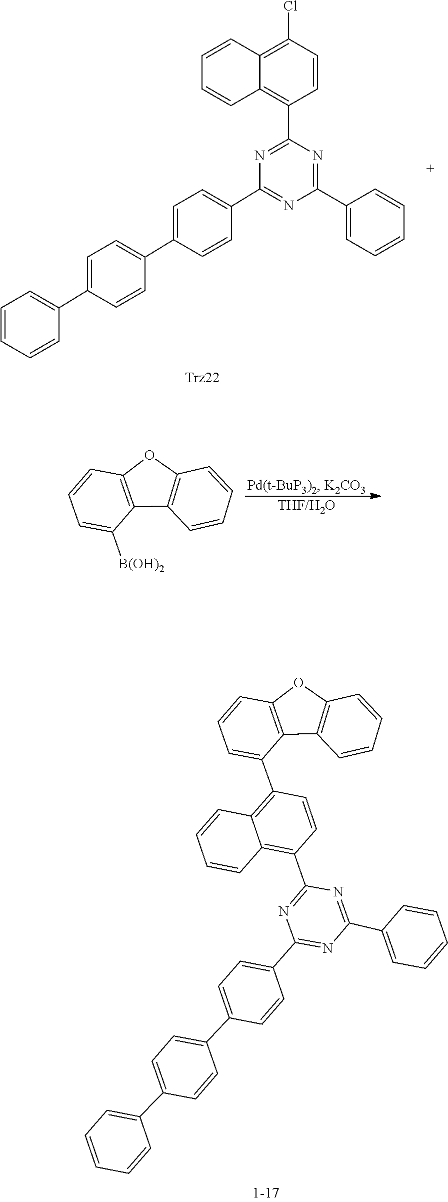 [SMILES: Clc1ccc(-c2nc(-c3ccccc3)nc(-c3ccc(-c4ccc(-c5ccccc5)cc4)cc3)n2)c2ccccc12 ; OB(O)c1cccc2oc3ccccc3c12 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

Trz22 (15 g, 27.5 mmol) and dibenzo[b,d]furan-1-ylboronic acid (6.1 g, 28.8 mmol) were added to 300 ml of THF, and the mixture was stirred and refluxed. Then, potassium carbonate (11.4 g, 82.4 mmol) was dissolved in 34 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14 g of Compound 1-17. (Yield: 75%, MS: [M+H]+=678)

### Synthesis Example 1-18

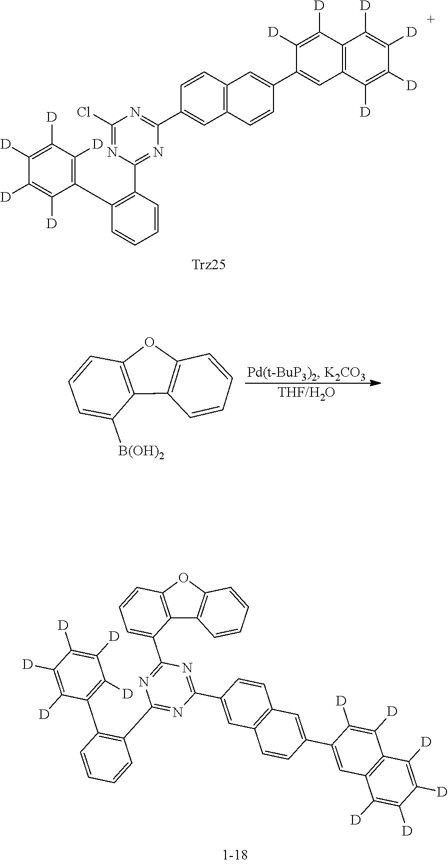 [SMILES: [2H]c1c([2H])c([2H])c(-c2ccccc2-c2nc(Cl)nc(-c3ccc4cc(-c5cc6c([2H])c([2H])c([2H])c([2H])c6c([2H])c5[2H])ccc4c3)n2)c([2H])c1[2H] ; OB(O)c1cccc2oc3ccccc3c12 ; [2H]c1c([2H])c([2H])c(-c2ccccc2-c2nc(-c3ccc4cc(-c5cc6c([2H])c([2H])c([2H])c([2H])c6c([2H])c5[2H])ccc4c3)nc(-c3cccc4oc5ccccc5c34)n2)c([2H])c1[2H]]

Trz25 (15 g, 28.2 mmol) and dibenzo[b,d]furan-1-ylboronic acid (6.3 g, 29.7 mmol) were added to 300 ml of THF, and the mixture was stirred and refluxed. Then, potassium carbonate (11.7 g, 84.7 mmol) was dissolved in 35 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.9 g of Compound 1-18. (Yield: 69%, MS: [M+H]+=663)

### Synthesis Example 1-19

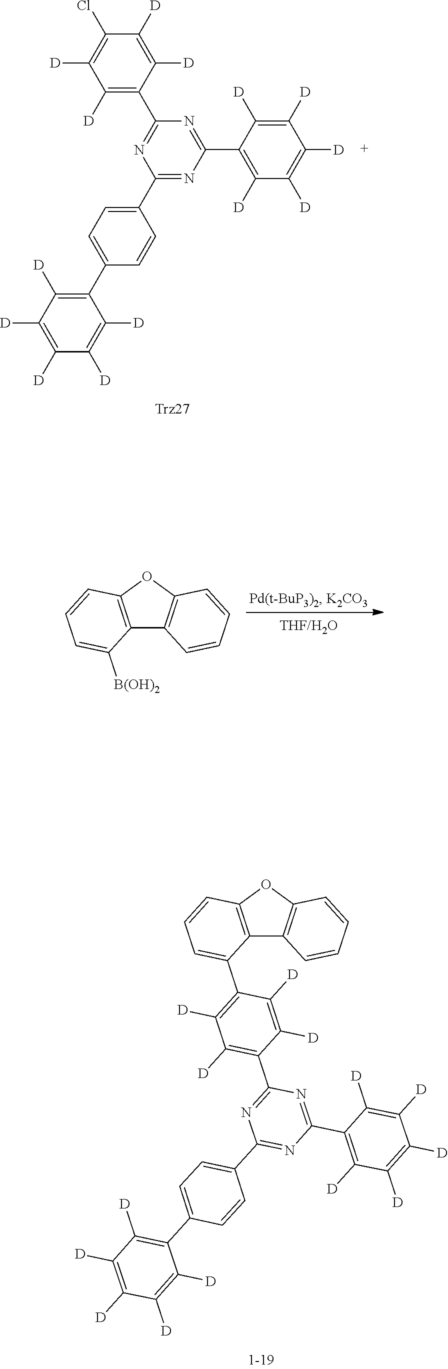 [SMILES: [2H]c1c([2H])c([2H])c(-c2ccc(-c3nc(-c4c([2H])c([2H])c([2H])c([2H])c4[2H])nc(-c4c([2H])c([2H])c(Cl)c([2H])c4[2H])n3)cc2)c([2H])c1[2H] ; OB(O)c1cccc2oc3ccccc3c12 ; [2H]c1c([2H])c([2H])c(-c2ccc(-c3nc(-c4c([2H])c([2H])c([2H])c([2H])c4[2H])nc(-c4c([2H])c([2H])c(-c5cccc6oc7ccccc7c56)c([2H])c4[2H])n3)cc2)c([2H])c1[2H]]

Trz27 (15 g, 34.6 mmol) and dibenzo[b,d]furan-1-ylboronic acid (7.7 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.9 g of Compound 1-19. (Yield: 71%, MS: [M+H]+=566)

### Synthesis Example 1-20

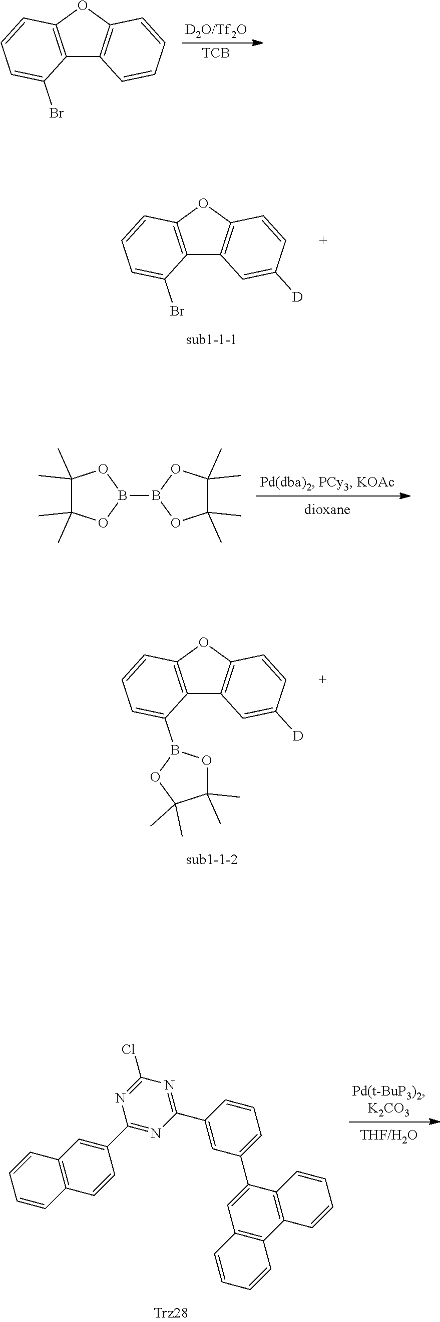

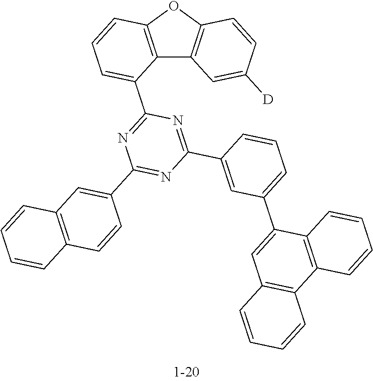 [SMILES: [2H]c1ccc2oc3cccc(-c4nc(-c5cccc(-c6cc7ccccc7c7ccccc67)c5)nc(-c5ccc6ccccc6c5)n4)c3c2c1]

Trifluoromethanesulfonic anhydride (24 g, 85 mmol) and deuterium oxide (8.5 g, 424.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 5 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.7 g of compound sub1-1-1. (Yield: 38%, MS: [M+H]+=248)

sub1-1-1 (15 g, 60.5 mmol) and bis(pinacolato)diboron (16.9 g, 66.5 mmol) were added to 300 ml of 1,4-dioxane, and the mixture was stirred under reflux. Then, potassium acetate (8.9 g, 90.7 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 13.4 g of sub1-1-2. (Yield: 75%, MS: [M+H]+=296)

sub1-1-2 (15 g, 50.8 mmol) and Trz28 (26.4 g, 53.4 mmol) were added to 300 ml of THF, and the mixture was stirred and refluxed. Then, potassium carbonate (21.1 g, 152.5 mmol) was dissolved in 63 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21 g of Compound 1-20. (Yield: 66%, MS: [M+H]+=627)

### Synthesis Example 1-21

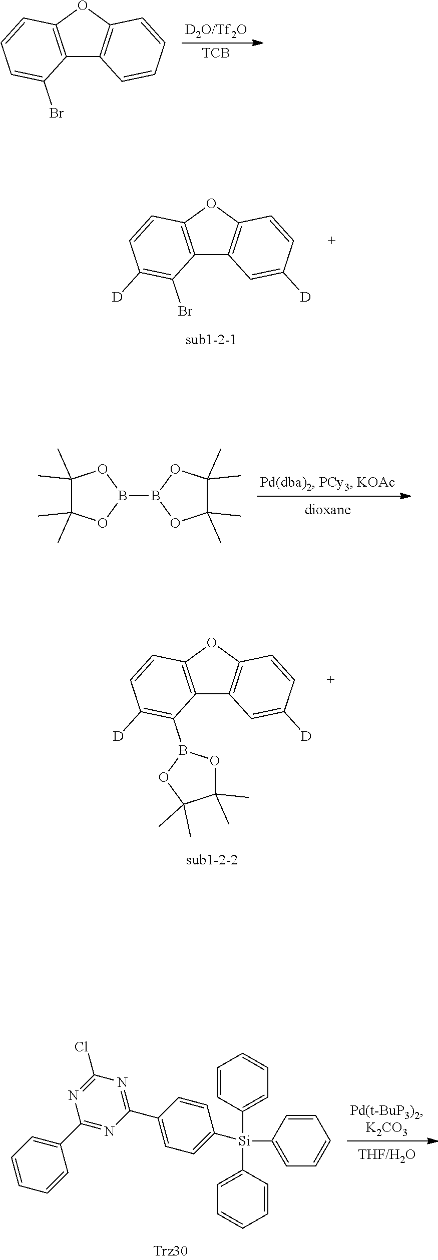

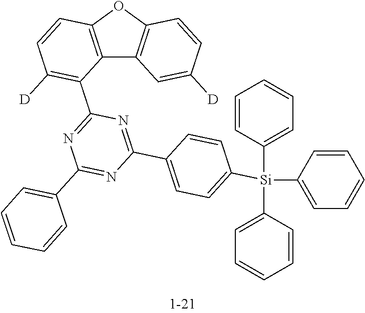 [SMILES: [2H]c1ccc2oc3ccc([2H])c(-c4nc(-c5ccccc5)nc(-c5ccc([Si](c6ccccc6)(c6ccccc6)c6ccccc6)cc5)n4)c3c2c1]

Trifluoromethanesulfonic anhydride (48 g, 170 mmol) and deuterium oxide (17 g, 849.9 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 8 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of compound sub1-2-1. (Yield: 40%, MS: [M+H]+=249)

sub1-2-1 (15 g, 60.2 mmol) and bis(pinacolato)diboron (16.8 g, 66.2 mmol) were added to 300 ml of 1,4-dioxane, and the mixture was stirred under reflux. Then, potassium acetate (8.9 g, 90.3 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12.5 g of sub1-2-2. (Yield: 70%, MS: [M+H]+=297)

sub1-2-2 (15 g, 50.6 mmol) and Trz30 (28 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-21. (Yield: 70%, MS: [M+H]+=660)

### Synthesis Example 1-22

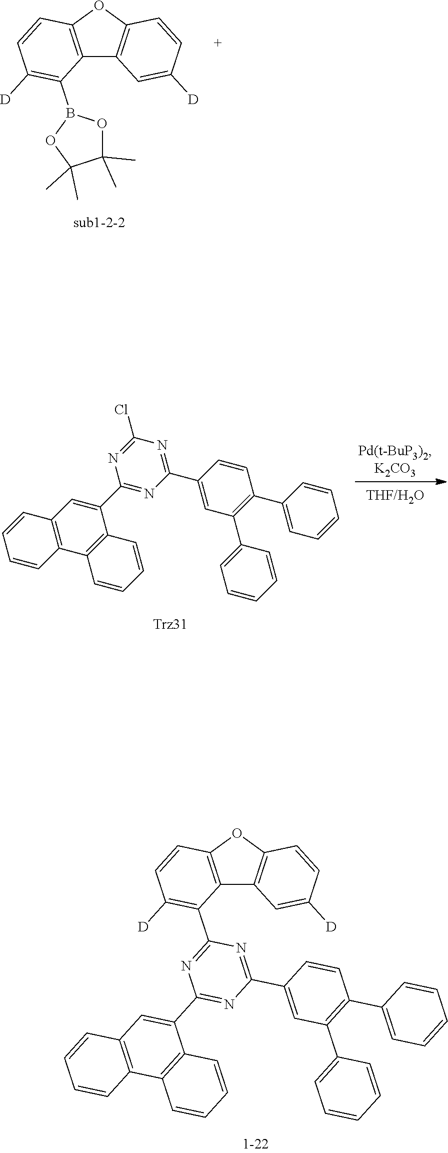 [SMILES: [2H]c1ccc2oc3ccc([2H])c(B4OC(C)(C)C(C)(C)O4)c3c2c1 ; Clc1nc(-c2ccc(-c3ccccc3)c(-c3ccccc3)c2)nc(-c2cc3ccccc3c3ccccc23)n1 ; [2H]c1ccc2oc3ccc([2H])c(-c4nc(-c5ccc(-c6ccccc6)c(-c6ccccc6)c5)nc(-c5cc6ccccc6c6ccccc56)n4)c3c2c1]

sub1-2-2 (15 g, 50.6 mmol) and Trz31 (21.9 g, 53.2 mmol) were added to 300 ml of THF, and the mixture was stirred and refluxed. Then, potassium carbonate (21 g, 151.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.5 g of Compound 1-22. (Yield: 68%, MS: [M+H]+=654)

### Synthesis Example 1-23

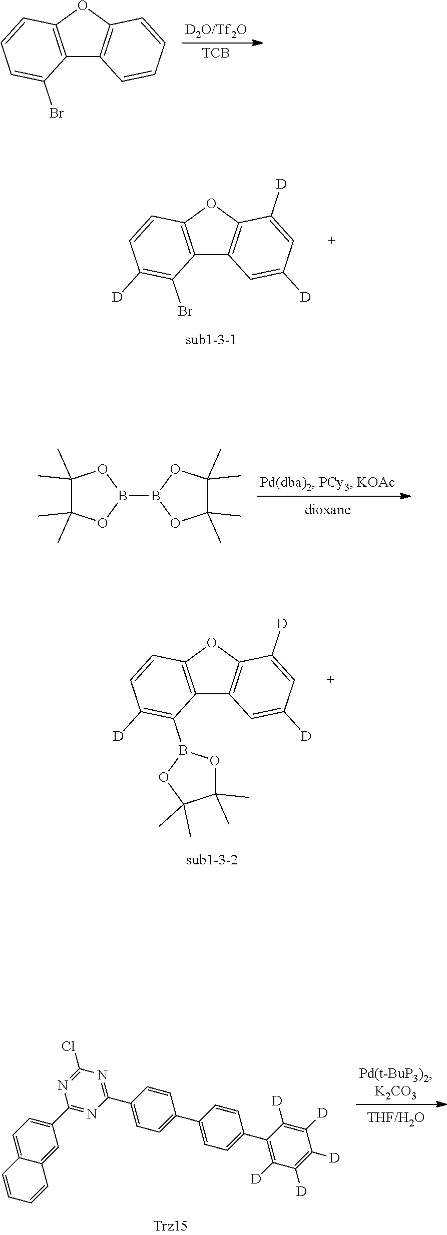

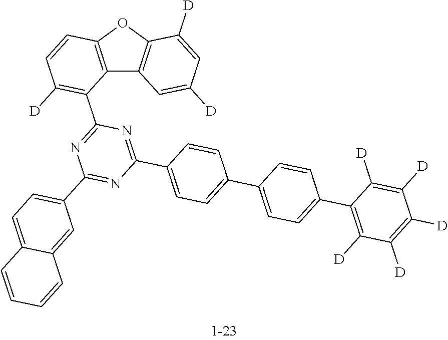 [SMILES: [2H]c1cc([2H])c2oc3ccc([2H])c(-c4nc(-c5ccc(-c6ccc(-c7c([2H])c([2H])c([2H])c([2H])c7[2H])cc6)cc5)nc(-c5ccc6ccccc6c5)n4)c3c2c1]

Trifluoromethanesulfonic anhydride (71.9 g, 255 mmol) and deuterium oxide (25.5 g, 1274.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 14 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.3 g of compound sub1-3-1. (Yield: 42%, MS: [M+H]+=250)

sub1-3-1 (15 g, 60 mmol) and bis(pinacolato)diboron (16.8 g, 66 mmol) were added to 300 ml of 1,4-dioxane, and the mixture was stirred under reflux. Then, potassium acetate (8.8 g, 90 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.4 g of sub1-3-2. (Yield: 64%, MS: [M+H]+=298)

sub1-3-2 (15 g, 50.5 mmol) and Trz15 (25.2 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.1 g of Compound 1-23. (Yield: 75%, MS: [M+H]+=610)

### Synthesis Example 1-24

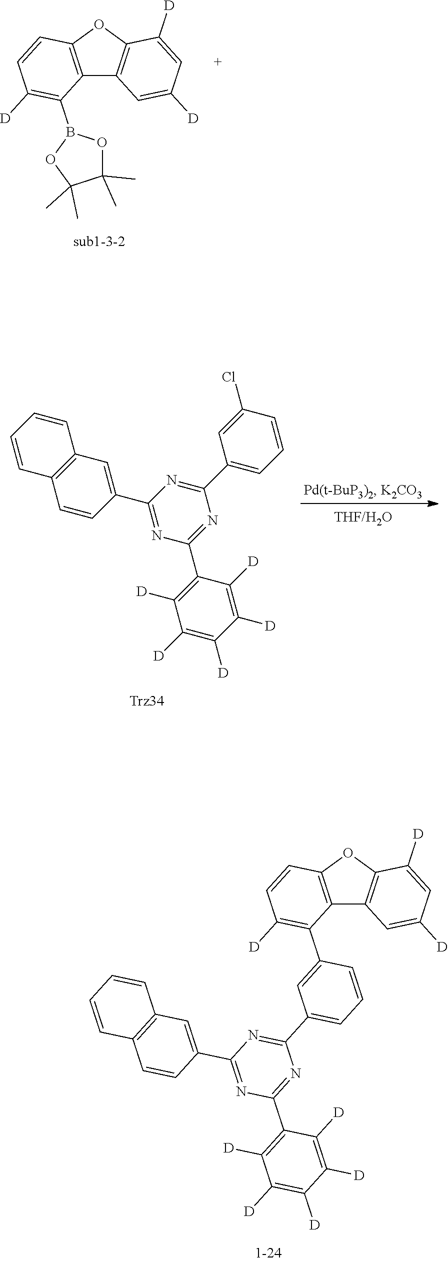 [SMILES: [2H]c1cc([2H])c2oc3ccc([2H])c(B4OC(C)(C)C(C)(C)O4)c3c2c1 ; [2H]c1c([2H])c([2H])c(-c2nc(-c3cccc(Cl)c3)nc(-c3ccc4ccccc4c3)n2)c([2H])c1[2H] ; [2H]c1cc([2H])c2oc3ccc([2H])c(-c4cccc(-c5nc(-c6ccc7ccccc7c6)nc(-c6c([2H])c([2H])c([2H])c([2H])c6[2H])n5)c4)c3c2c1]

sub1-3-2 (15 g, 50.5 mmol) and Trz34 (21.1 g, 53 mmol) were added to 300 ml of THF, and the mixture was stirred and refluxed. Then, potassium carbonate (20.9 g, 151.4 mmol) was dissolved in 63 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.8 g of Compound 1-24. (Yield: 66%, MS: [M+H]+=534)

### Synthesis Example 1-25

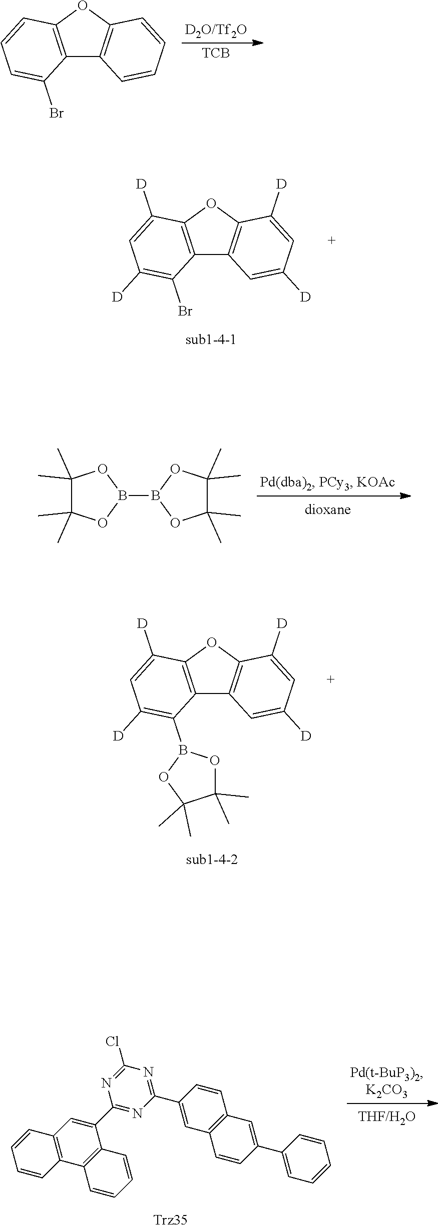

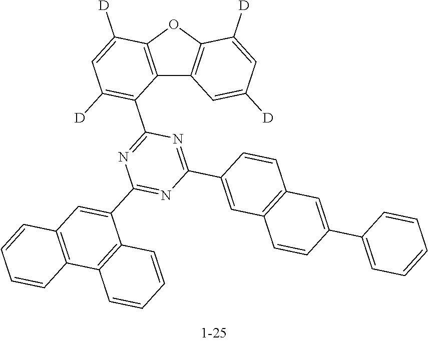 [SMILES: [2H]c1cc([2H])c2oc3c([2H])cc([2H])c(-c4nc(-c5ccc6cc(-c7ccccc7)ccc6c5)nc(-c5cc6ccccc6c6ccccc56)n4)c3c2c1]

Trifluoromethanesulfonic anhydride (95.9 g, 340 mmol) and Deuterium oxide (34 g, 1699.8 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 20 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of compound sub1-4-1. (Yield: 37%, MS: [M+H]+=251)

sub1-4-1 (15 g, 59.7 mmol) and bis(pinacolato)diboron (16.7 g, 65.7 mmol) were added to 300 ml of 1,4-dioxane, and the mixture was stirred under reflux. Then, potassium acetate (8.8 g, 89.6 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12.5 g of sub1-4-2. (Yield: 70%, MS: [M+H]+=299)

sub1-4-2 (15 g, 50.3 mmol) and Trz35 (26.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.5 g of Compound 1-25. (Yield: 68%, MS: [M+H]+=631)

### Synthesis Example 1-26

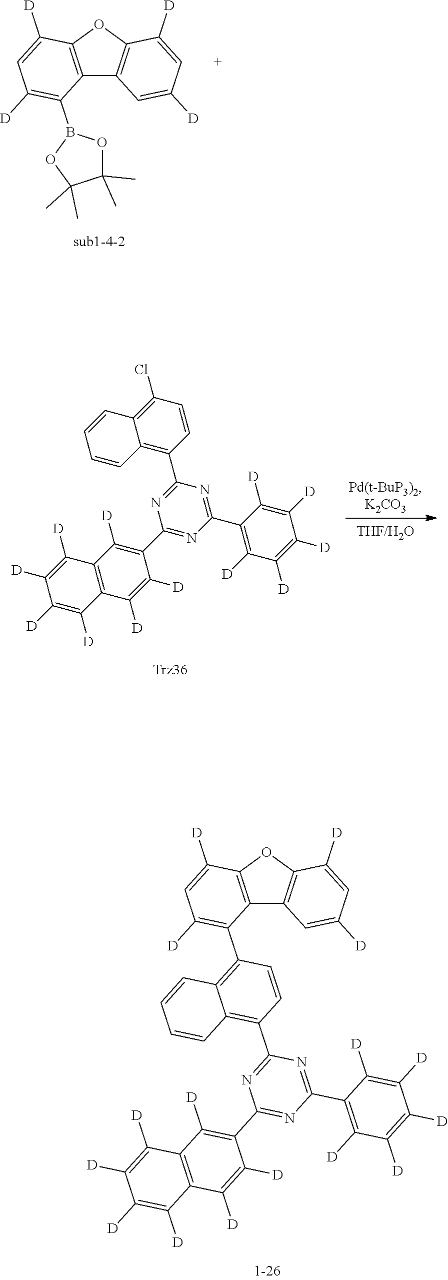 [SMILES: [2H]c1cc([2H])c2oc3c([2H])cc([2H])c(B4OC(C)(C)C(C)(C)O4)c3c2c1 ; [2H]c1c([2H])c([2H])c(-c2nc(-c3c([2H])c([2H])c4c([2H])c([2H])c([2H])c([2H])c4c3[2H])nc(-c3ccc(Cl)c4ccccc34)n2)c([2H])c1[2H] ; [2H]c1cc([2H])c2oc3c([2H])cc([2H])c(-c4ccc(-c5nc(-c6c([2H])c([2H])c([2H])c([2H])c6[2H])nc(-c6c([2H])c([2H])c7c([2H])c([2H])c([2H])c([2H])c7c6[2H])n5)c5ccccc45)c3c2c1]

sub1-4-2 (15 g, 50.3 mmol) and Trz36 (24.1 g, 52.8 mmol) were added to 300 ml of THF, and the mixture was stirred and refluxed. Then, potassium carbonate (20.9 g, 150.9 mmol) was dissolved in 63 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-26. (Yield: 68%, MS: [M+H]+=592)

### Synthesis Example 1-27

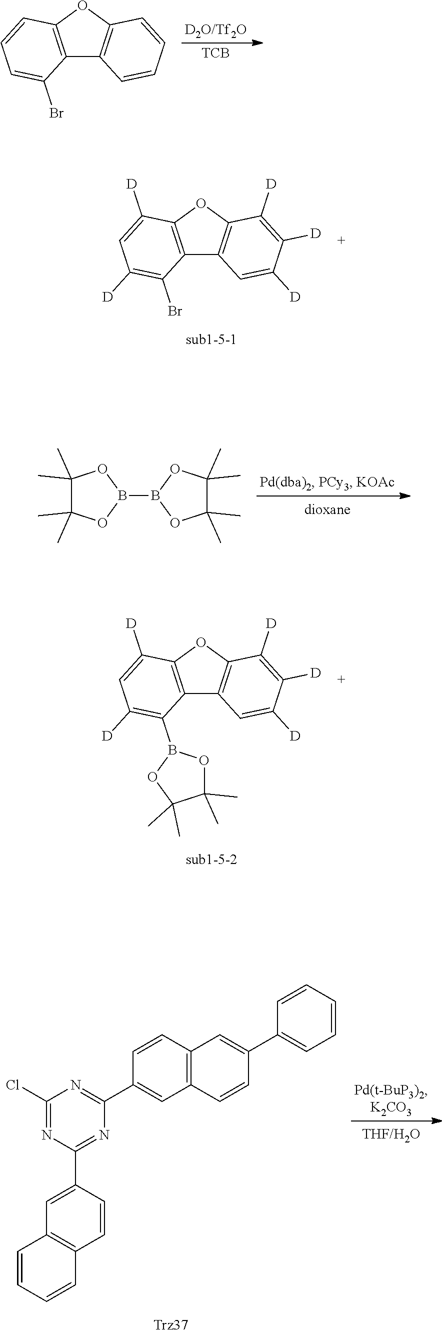

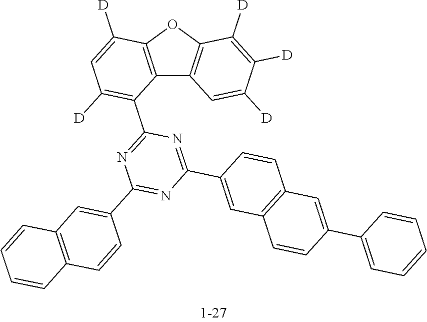 [SMILES: [2H]c1cc2c(oc3c([2H])cc([2H])c(-c4nc(-c5ccc6ccccc6c5)nc(-c5ccc6cc(-c7ccccc7)ccc6c5)n4)c32)c([2H])c1[2H]]

Trifluoromethanesulfonic anhydride (119.9 g, 424.9 mmol) and deuterium oxide (42.6 g, 2124.7 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 24 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.9 g of compound sub1-5-1. (Yield: 39%, MS: [M+H]+=252)

sub1-5-1 (15 g, 59.5 mmol) and bis(pinacolato)diboron (16.6 g, 65.4 mmol) were added to 300 ml of 1,4-dioxane, and the mixture was stirred under reflux. Then, potassium acetate (8.8 g, 89.2 mmol) was added thereto, sufficiently stirred, and then bis(dibenzylideneacetone)palladium(0) (1 g, 1.8 mmol) and tricyclohexylphosphine (1 g, 3.6 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.2 g of sub1-5-2. (Yield: 63%, MS: [M+H]+=300)

sub1-5-2 (15 g, 50.1 mmol) and Trz37 (23.4 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.1 g of Compound 1-27. (Yield: 69%, MS: [M+H]+=581)

### Synthesis Example 1-28

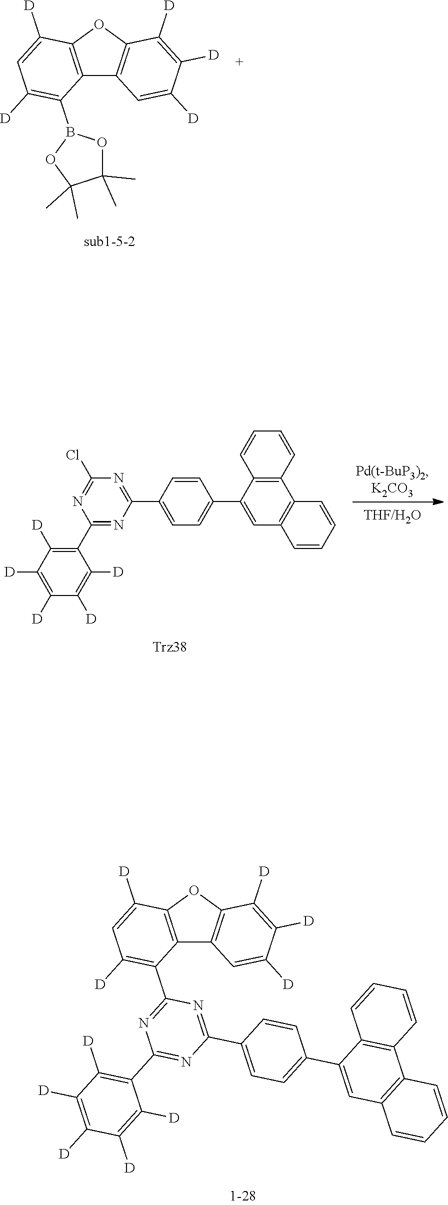 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2nc(Cl)nc(-c3ccc(-c4cc5ccccc5c5ccccc45)cc3)n2)c([2H])c1[2H] ; [2H]c1cc2c(oc3c([2H])cc([2H])c(-c4nc(-c5ccc(-c6cc7ccccc7c7ccccc67)cc5)nc(-c5c([2H])c([2H])c([2H])c([2H])c5[2H])n4)c32)c([2H])c1[2H]]

sub1-5-2 (15 g, 50.1 mmol) and Trz38 (23.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-28. (Yield: 69%, MS: [M+H]+=586)

### Synthesis Example 1-29

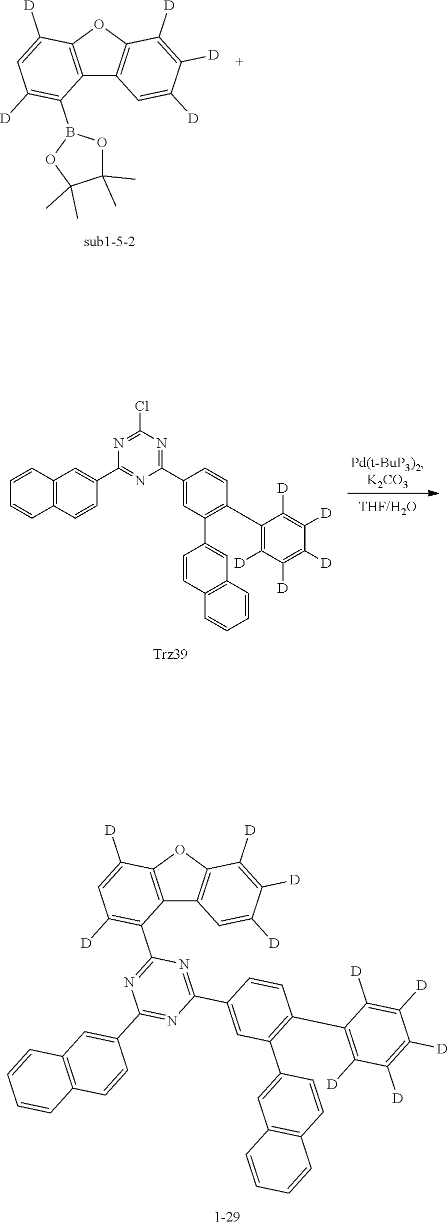 [SMILES: [2H]c1cc2c(oc3c([2H])cc([2H])c(B4OC(C)(C)C(C)(C)O4)c32)c([2H])c1[2H] ; [2H]c1c([2H])c([2H])c(-c2ccc(-c3nc(Cl)nc(-c4ccc5ccccc5c4)n3)cc2-c2ccc3ccccc3c2)c([2H])c1[2H] ; [2H]c1cc2c(oc3c([2H])cc([2H])c(-c4nc(-c5ccc(-c6c([2H])c([2H])c([2H])c([2H])c6[2H])c(-c6ccc7ccccc7c6)c5)nc(-c5ccc6ccccc6c5)n4)c32)c([2H])c1[2H]]

sub1-5-2 (15 g, 50.1 mmol) and Trz39 (27.6 g, 52.6 mmol) were added to 300 ml of THF, and the mixture was stirred and refluxed. Then, potassium carbonate (20.8 g, 150.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.5 g of Compound 1-29. (Yield: 68%, MS: [M+H]+=662)

### Synthesis Example 1-30

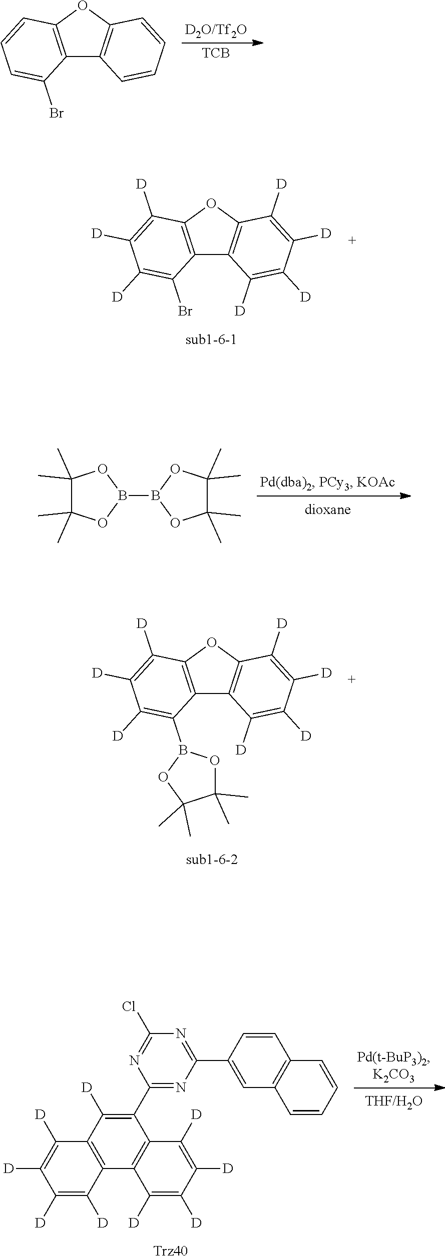

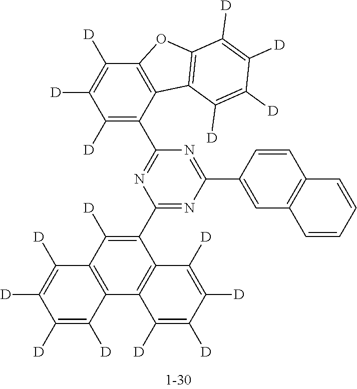 [SMILES: [2H]c1c([2H])c([2H])c2c(oc3c([2H])c([2H])c([2H])c(-c4nc(-c5ccc6ccccc6c5)nc(-c5c([2H])c6c([2H])c([2H])c([2H])c([2H])c6c6c([2H])c([2H])c([2H])c([2H])c56)n4)c32)c1[2H]]

Trifluoromethanesulfonic anhydride (167.8 g, 594.9 mmol) and deuterium oxide (59.6 g, 2974.6 mmol) were added at 0° C. and stirred for 5 hours to prepare a solution. 1-Bromodibenzo[b,d]furan (15 g, 60.7 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromodibenzo[b,d]furan and 1,2,4-trichlorobenzene, and the mixture was stirred while heating to 140° C. and then keeping that temperature. After the reaction for 36 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of compound sub1-6-1. (Yield: 40%, MS: [M+H]+=254)

sub1-6-1 (15 g, 59 mmol) and bis(pinacolato)diboron (16.5 g, 64.9 mmol) were added to 300 ml of 1,4-dioxane, and the mixture was stirred under reflux. Then, potassium acetate (8.7 g, 88.5 mmol) was added thereto, sufficiently stirred, and then bis(dibenzylideneacetone)palladium(0) (1 g, 1.8 mmol) and tricyclohexylphosphine (1 g, 3.5 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.6 g of sub1-6-2. (Yield: 65%, MS: [M+H]+=302)

sub1-6-2 (15 g, 49.8 mmol) and Trz 40 (22.3 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.3 g of Compound 1-30. (Yield: 72%, MS: [M+H]+=566)

### Synthesis Example 1-31

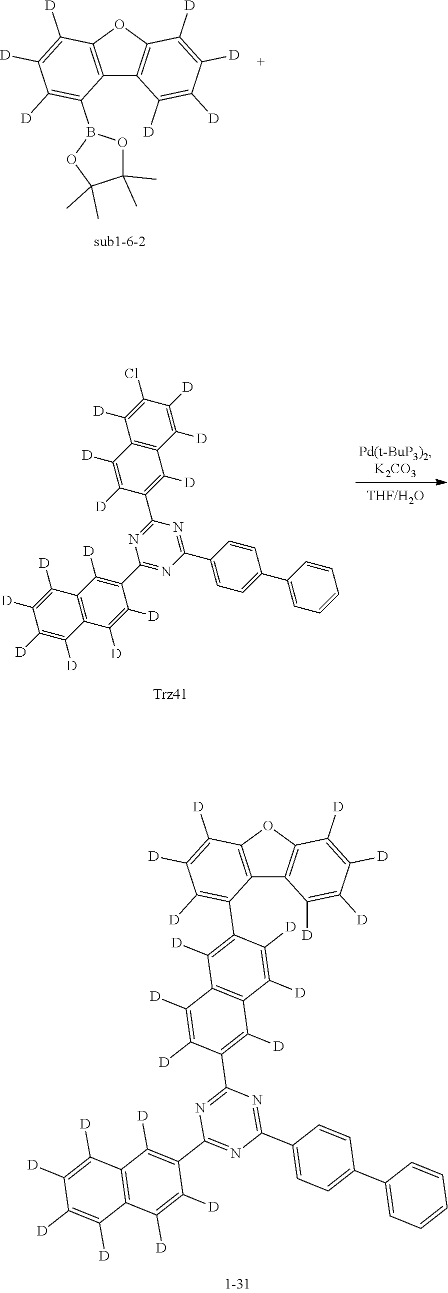 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c2c([2H])c(-c3nc(-c4ccc(-c5ccccc5)cc4)nc(-c4c([2H])c([2H])c5c([2H])c(Cl)c([2H])c([2H])c5c4[2H])n3)c([2H])c([2H])c2c1[2H] ; [2H]c1c([2H])c([2H])c2c(oc3c([2H])c([2H])c([2H])c(-c4c([2H])c([2H])c5c([2H])c(-c6nc(-c7ccc(-c8ccccc8)cc7)nc(-c7c([2H])c([2H])c8c([2H])c([2H])c([2H])c([2H])c8c7[2H])n6)c([2H])c([2H])c5c4[2H])c32)c1[2H]]

sub1-6-2 (15 g, 49.8 mmol) and Trz41 (27.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.7 g of Compound 1-31. (Yield: 74%, MS: [M+H]+=672)

### Synthesis Example 1-32

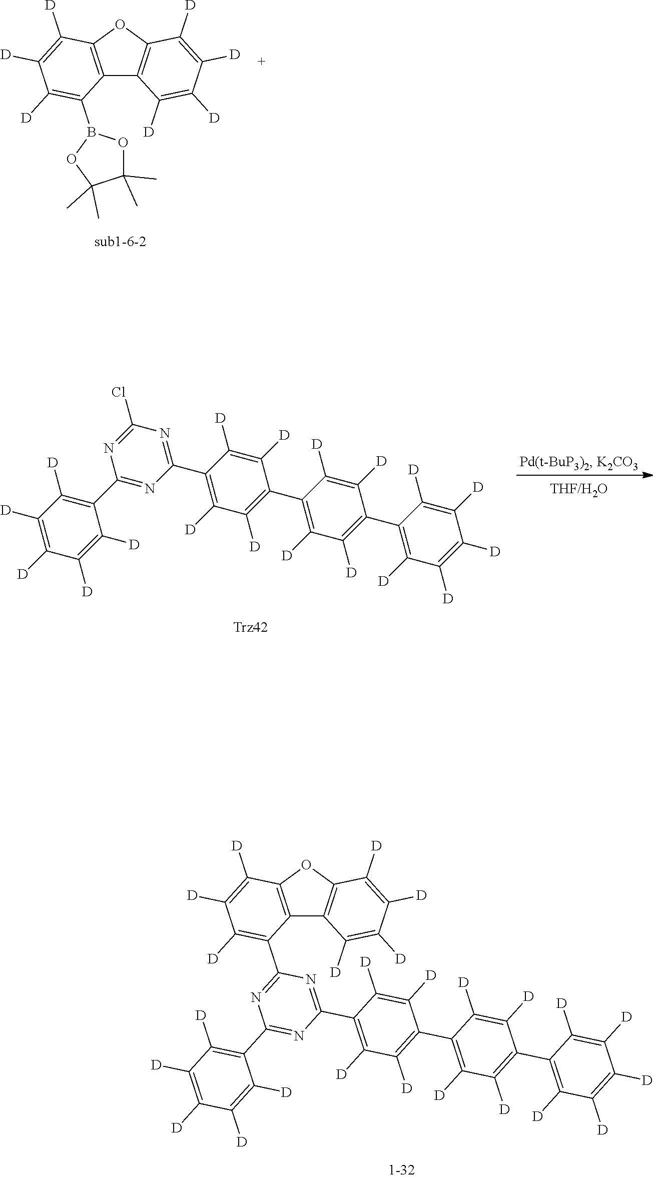 [SMILES: [2H]c1c([2H])c([2H])c2c(oc3c([2H])c([2H])c([2H])c(B4OC(C)(C)C(C)(C)O4)c32)c1[2H] ; [2H]c1c([2H])c([2H])c(-c2nc(Cl)nc(-c3c([2H])c([2H])c(-c4c([2H])c([2H])c(-c5c([2H])c([2H])c([2H])c([2H])c5[2H])c([2H])c4[2H])c([2H])c3[2H])n2)c([2H])c1[2H] ; [2H]c1c([2H])c([2H])c(-c2nc(-c3c([2H])c([2H])c(-c4c([2H])c([2H])c(-c5c([2H])c([2H])c([2H])c([2H])c5[2H])c([2H])c4[2H])c([2H])c3[2H])nc(-c3c([2H])c([2H])c([2H])c4oc5c([2H])c([2H])c([2H])c([2H])c5c34)n2)c([2H])c1[2H]]

sub1-6-2 (15 g, 49.8 mmol) and Trz42 (22.9 g, 52.3 mmol) were added to 300 ml of THF, and the mixture was stirred and refluxed. Then, potassium carbonate (20.6 g, 149.4 mmol) was dissolved in 62 ml of water and added thereto, and the mixture was sufficiently stirred and then bis(tri-tert-butylphosphine)palladium(0) (0.3 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.7 g of Compound 1-32. (Yield: 65%, MS: [M+H]+=577)

### Synthesis Example 1-33

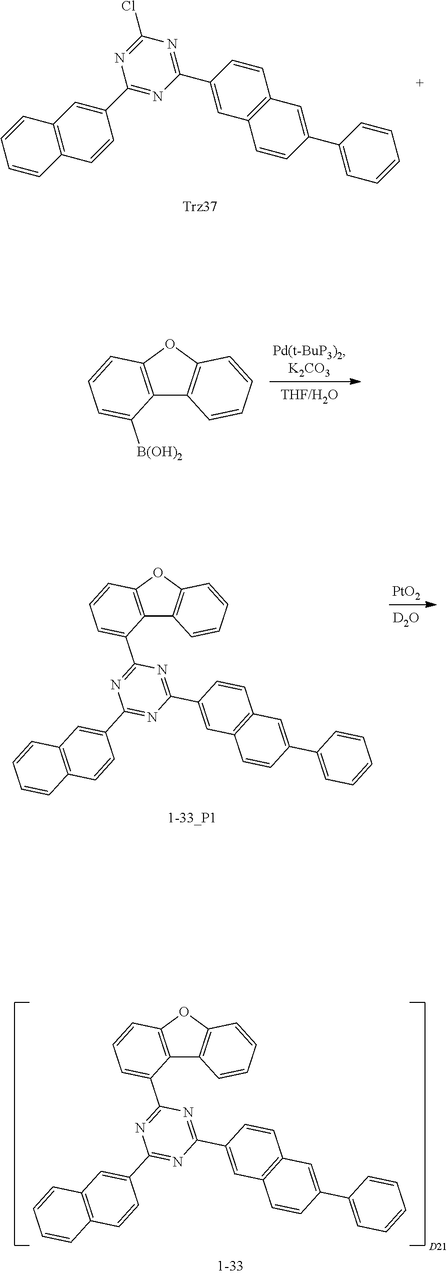

Trz37 (15 g, 33.8 mmol) and dibenzo[b,d]furan-1-ylboronic acid (7.5 g, 35.5 mmol) were added to 300 ml of THF, and the mixture was stirred and refluxed. Then, potassium carbonate (14 g, 101.4 mmol) was dissolved in 42 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.8 g of Compound 1-33_P1. (Yield: 66%, MS: [M+H]+=576)

Compound 1-33_P1 (10 g, 17.4 mmol), PtO₂ (1.2 g, 5.2 mmol) and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO4 and concentrated, and then the sample was purified by silica gel column chromatography to prepare 4.1 g of Compound 1-33. (Yield: 40%, MS; [M+H]+=598)

### Synthesis Example 1-34

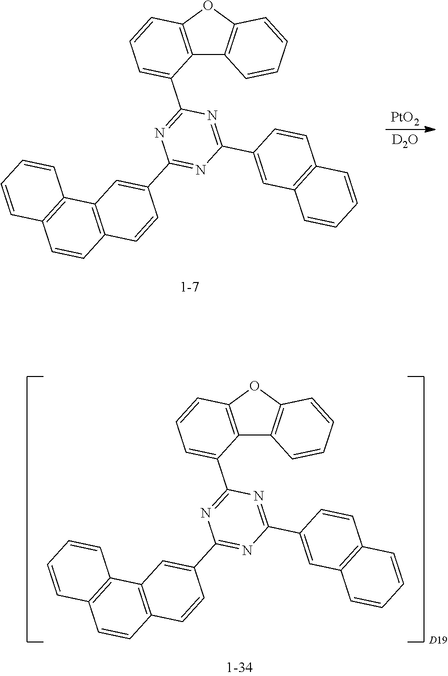

Compound 1-7 (10 g, 18.2 mmol), PtO₂ (1.2 g, 5.5 mmol) and D₂O (91 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 4.1 g of Compound 1-34. (Yield: 40%, MS: [M+H]+=570)

### Synthesis Example 1-35

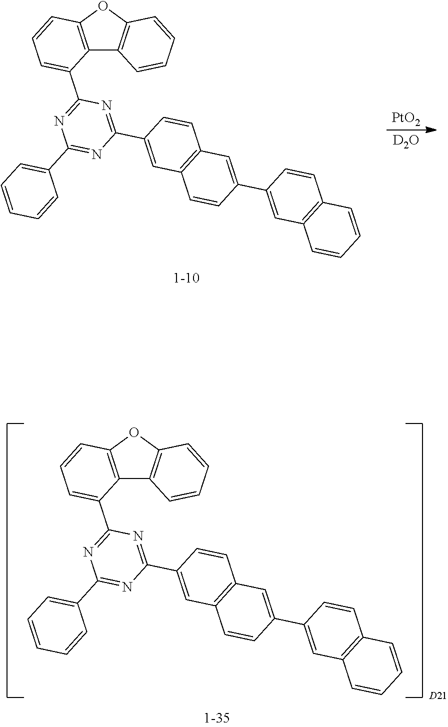

Compound 1-10 (10 g, 17.4 mmol), PtO₂ (1.2 g, 5.2 mmol) and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 4.5 g of Compound 1-35. (Yield: 43%, MS: [M+H]+=598)

### Synthesis Example 1-36

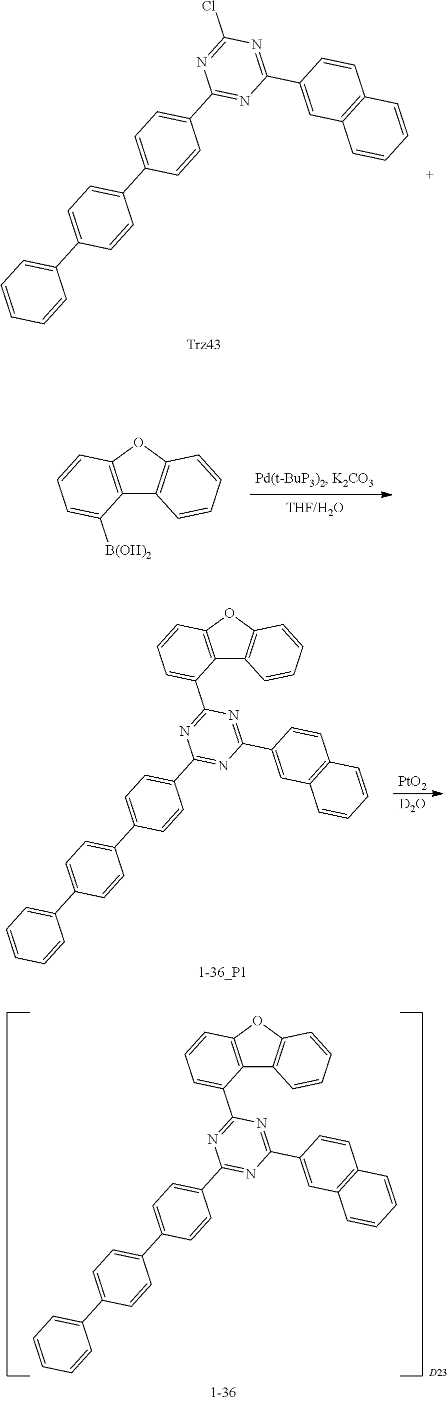

Trz43 (15 g, 31.9 mmol) and dibenzo[b,d]furan-1-ylboronic acid (7.1 g, 33.5 mmol) were added to 300 ml of THF, and the mixture was stirred and refluxed. Then, potassium carbonate (13.2 g, 95.8 mmol) was dissolved in 40 ml of water and added thereto, and the mixture was sufficiently stirred and then bis(tri-tert-butylphosphine)palladium(0) (0.2 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-36_P1. (Yield: 74%, MS: [M+H]+=602)

Compound 1-36_P1 (10 g, 16.6 mmol), PtO₂ (1.1 g, 5 mmol) and D₂O (83 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 4.5 g of Compound 1-36. (Yield: 43%, MS: [M+H]+=626)

### Synthesis Example 1-37

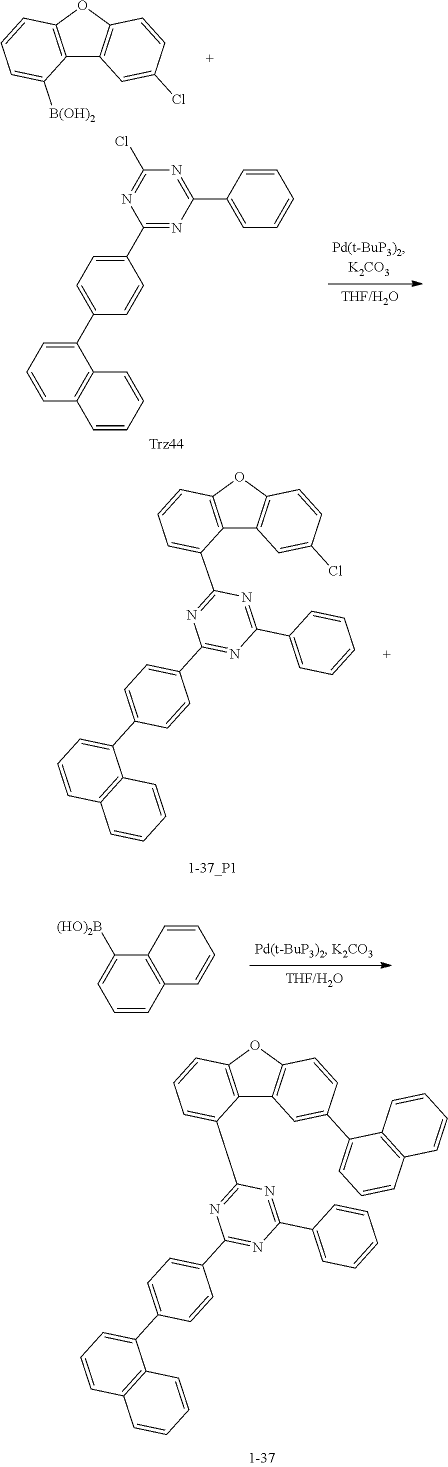 [SMILES: OB(O)c1cccc2oc3ccc(Cl)cc3c12 ; Clc1nc(-c2ccccc2)nc(-c2ccc(-c3cccc4ccccc34)cc2)n1 ; Clc1ccc2oc3cccc(-c4nc(-c5ccccc5)nc(-c5ccc(-c6cccc7ccccc67)cc5)n4)c3c2c1 ; OB(O)c1cccc2ccccc12 ; c1ccc(-c2nc(-c3ccc(-c4cccc5ccccc45)cc3)nc(-c3cccc4oc5ccc(-c6cccc7ccccc67)cc5c34)n2)cc1]

(8-Chlorodibenzo[b,d]furan-1-yl)boronic acid (15 g, 60.9 mmol) and Trz44 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.5 g of Compound 1-37_P1. (Yield: 69%, MS: [M+H]+=560)

Compound 1-37_P1 (15 g, 26.8 mmol) and naphthalen-1-ylboronic acid (4.8 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.7 g of Compound 1-37. (Yield: 73%, MS: [M+H]+=652)

### Synthesis Example 1-38

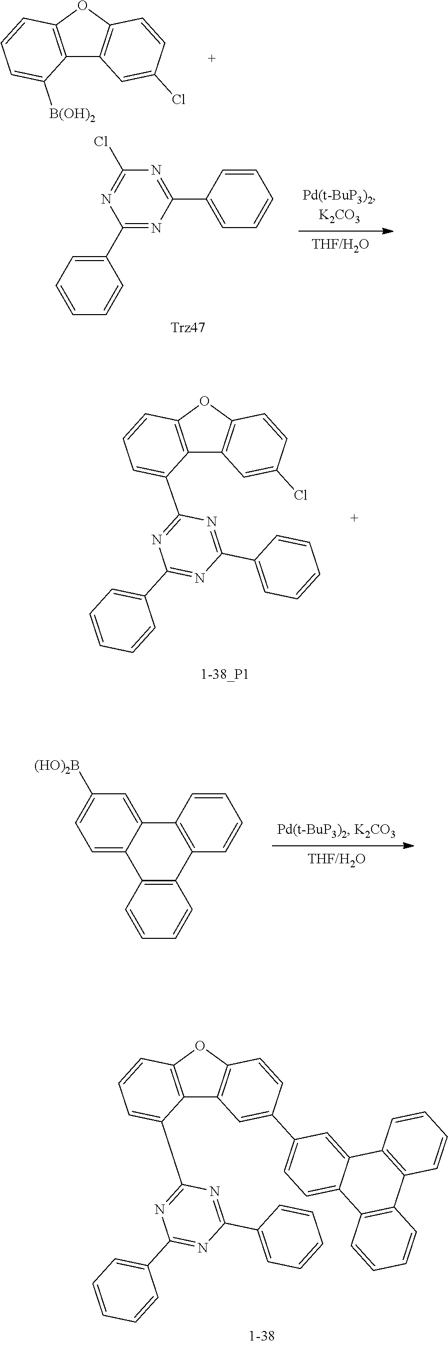 [SMILES: OB(O)c1cccc2oc3ccc(Cl)cc3c12 ; Clc1nc(-c2ccccc2)nc(-c2ccccc2)n1 ; Clc1ccc2oc3cccc(-c4nc(-c5ccccc5)nc(-c5ccccc5)n4)c3c2c1 ; OB(O)c1ccc2c3ccccc3c3ccccc3c2c1 ; c1ccc(-c2nc(-c3ccccc3)nc(-c3cccc4oc5ccc(-c6ccc7c8ccccc8c8ccccc8c7c6)cc5c34)n2)cc1]

(8-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.9 g of Compound 1-38_P1. (Yield: 68%, MS: [M+H]+=434)

Compound 1-38_P1 (15 g, 34.6 mmol) and triphenylen-2-ylboronic acid (9.9 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.3 g of Compound 1-38. (Yield: 66%, MS: [M+H]+=626)

### Synthesis Example 1-39

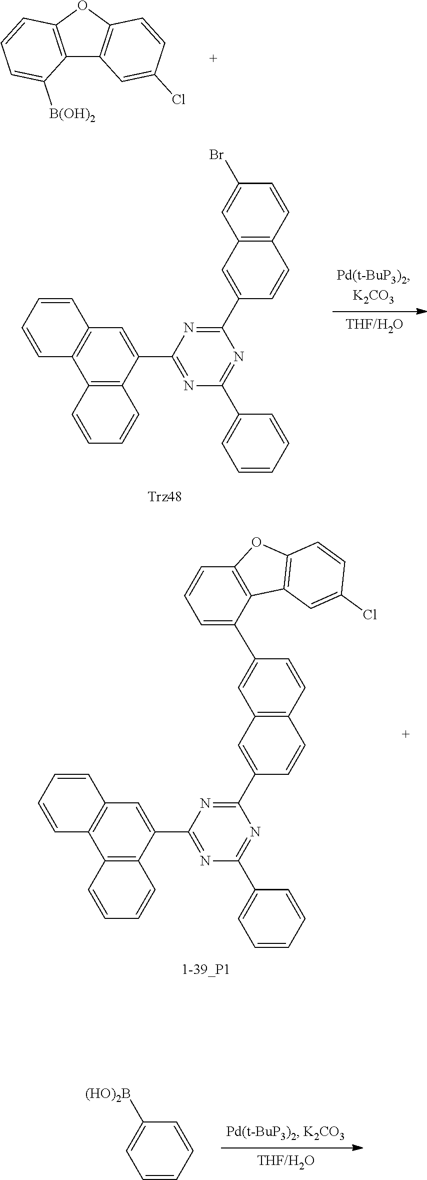 [SMILES: OB(O)c1cccc2oc3ccc(Cl)cc3c12 ; Brc1ccc2ccc(-c3nc(-c4ccccc4)nc(-c4cc5ccccc5c5ccccc45)n3)cc2c1 ; Clc1ccc2oc3cccc(-c4ccc5ccc(-c6nc(-c7ccccc7)nc(-c7cc8ccccc8c8ccccc78)n6)cc5c4)c3c2c1 ; OB(O)c1ccccc1]

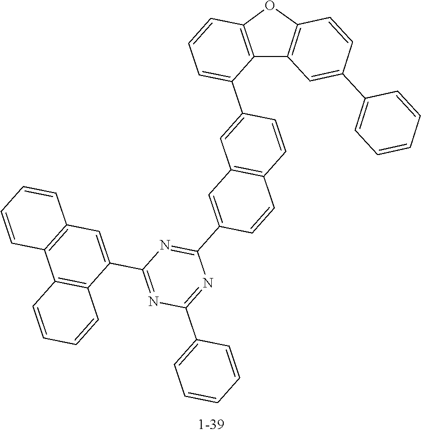 [SMILES: c1ccc(-c2ccc3oc4cccc(-c5ccc6ccc(-c7nc(-c8ccccc8)nc(-c8cc9ccccc9c9ccccc89)n7)cc6c5)c4c3c2)cc1]

(8-Chlorodibenzo[b,d]furan-1-yl)boronic acid (15 g, 60.9 mmol) and Trz48 (34.4 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.1 g of Compound 1-39_P1. (Yield: 75%, MS: [M+H]+=660)

Compound 1-39_P1 (15 g, 22.7 mmol) and phenylboronic acid (2.9 g, 23.9 mmol) were added to 300 ml of THF, and the mixture was stirred and refluxed. Then, potassium carbonate (9.4 g, 68.2 mmol) was dissolved in 28 ml of water and added thereto, and the mixture was sufficiently stirred and then bis(tri-tert-butylphosphine)palladium(0) (0.1 g, 0.2 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.7 g of Compound 1-39. (Yield: 67%, MS: [M+H]+=702)

### Synthesis Example 1-40

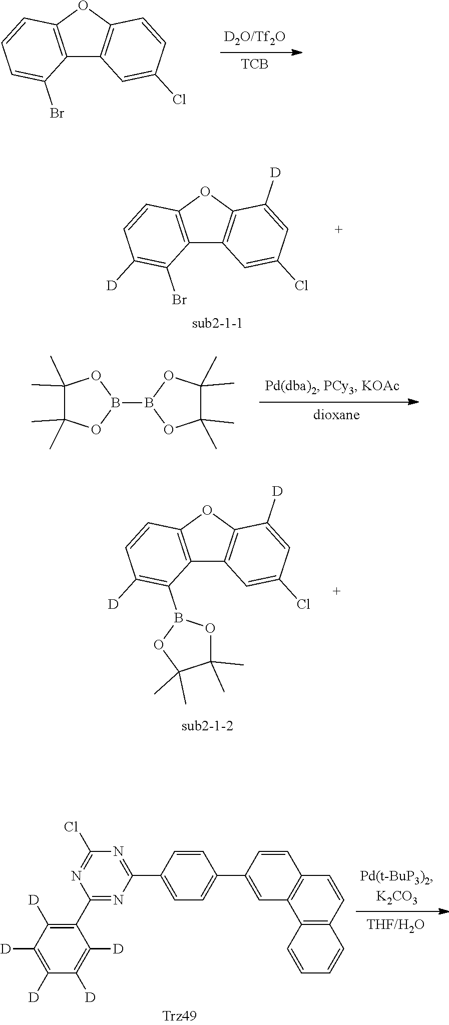

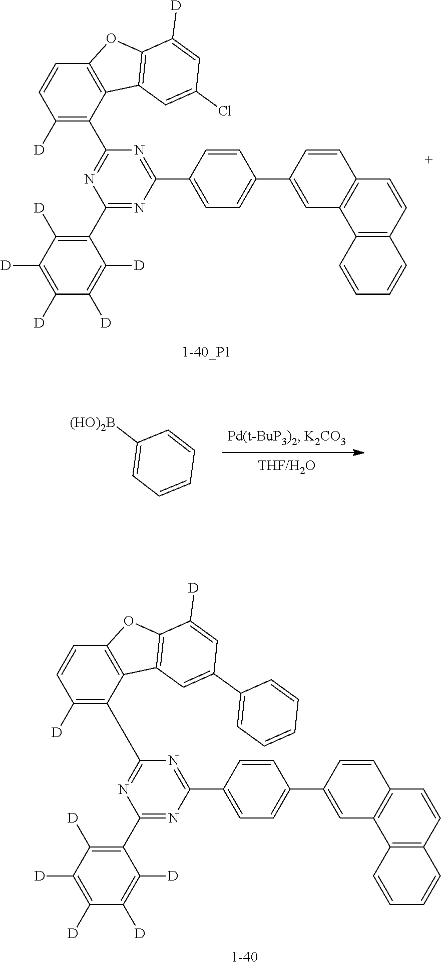 [SMILES: [2H]c1ccc2oc3c([2H])cc(Cl)cc3c2c1-c1nc(-c2ccc(-c3ccc4ccc5ccccc5c4c3)cc2)nc(-c2c([2H])c([2H])c([2H])c([2H])c2[2H])n1 ; OB(O)c1ccccc1 ; [2H]c1ccc2oc3c([2H])cc(-c4ccccc4)cc3c2c1-c1nc(-c2ccc(-c3ccc4ccc5ccccc5c4c3)cc2)nc(-c2c([2H])c([2H])c([2H])c([2H])c2[2H])n1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-8-chlorodibenzo[b,d]furan (15 g, 53.3 mmol) was added to 120 ml of 1,2,4-trichlorobenzene, and the mixture 10 was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of compound sub2-1-1. (Yield: 43%, MS: [M+H]+=283)

Sub2-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.5 g of sub2-1-2. (Yield: 66%, MS: [M+H]+=331)

Sub2-1-2 (15 g, 45.4 mmol) and Trz49 (21.4 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.2 g of Compound 1-40_P1. (Yield: 65%, MS: [M+H]+=617)

Compound 1-40_P1 (15 g, 24.3 mmol) and phenylboronic acid (3.1 g, 25.5 mmol) were added to 300 ml of THF, and the mixture was stirred and refluxed. Then, potassium carbonate (10.1 g, 72.9 mmol) was dissolved in 30 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11 g of Compound 1-40. (Yield: 69%, MS: [M+H]+=659)

### Synthesis Example 1-41

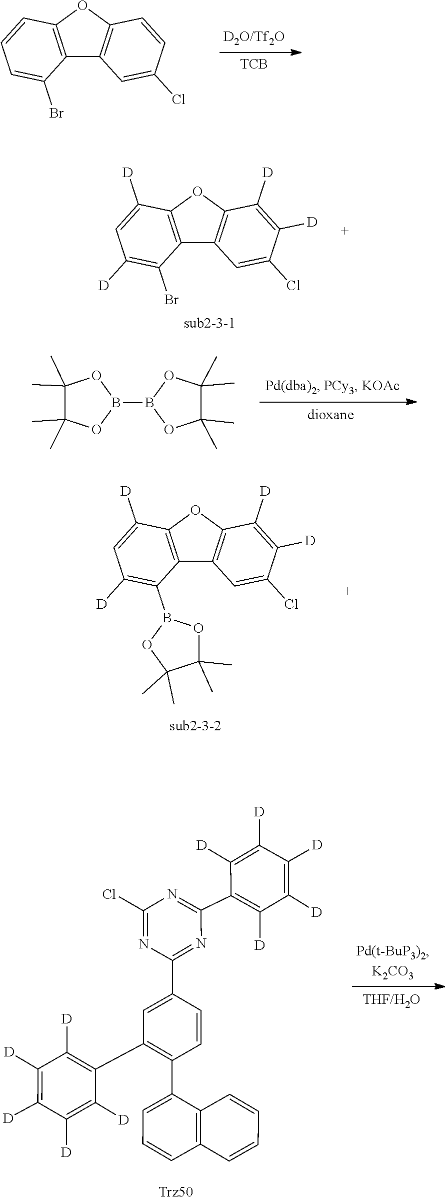

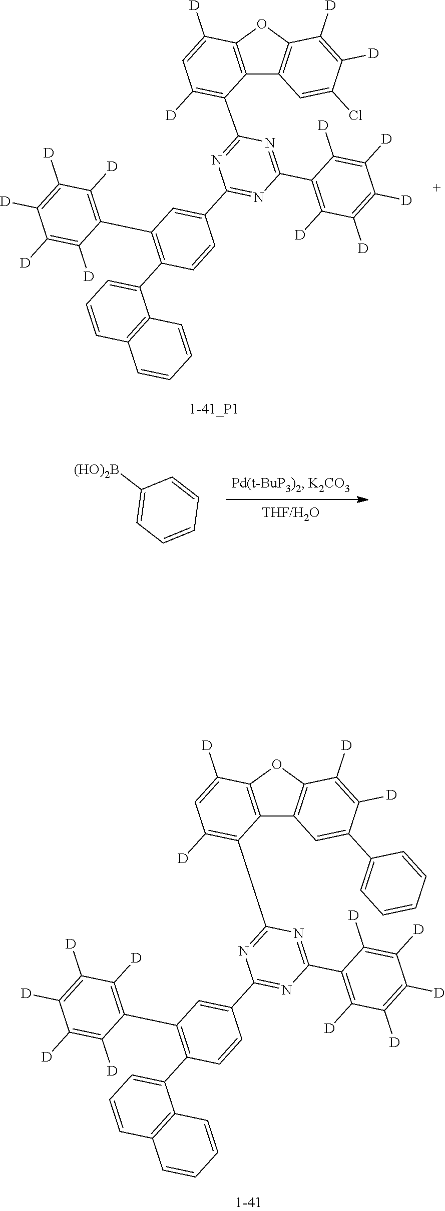 [SMILES: [2H]c1cc([2H])c2oc3c([2H])c([2H])c(Cl)cc3c2c1-c1nc(-c2ccc(-c3cccc4ccccc34)c(-c3c([2H])c([2H])c([2H])c([2H])c3[2H])c2)nc(-c2c([2H])c([2H])c([2H])c([2H])c2[2H])n1 ; OB(O)c1ccccc1 ; [2H]c1cc([2H])c2oc3c([2H])c([2H])c(-c4ccccc4)cc3c2c1-c1nc(-c2ccc(-c3cccc4ccccc34)c(-c3c([2H])c([2H])c([2H])c([2H])c3[2H])c2)nc(-c2c([2H])c([2H])c([2H])c([2H])c2[2H])n1]

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-8-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-8-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.4 g of compound sub2-3-1. (Yield: 42%, MS: [M+H]+=285)

Sub2-3-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12 g of sub2-3-2. (Yield: 69%, MS: [M+H]+=333)

Sub2-3-2 (15 g, 45.1 mmol) and Trz50 (22.7 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-41_P1. (Yield: 69%, MS: [M+H]+=650)

Compound 1-41_P1 (15 g, 23.1 mmol) and phenylboronic acid (2.9 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.5 g of Compound 1-41. (Yield: 66%, MS: [M+H]+=692)

### Synthesis Example 1-42

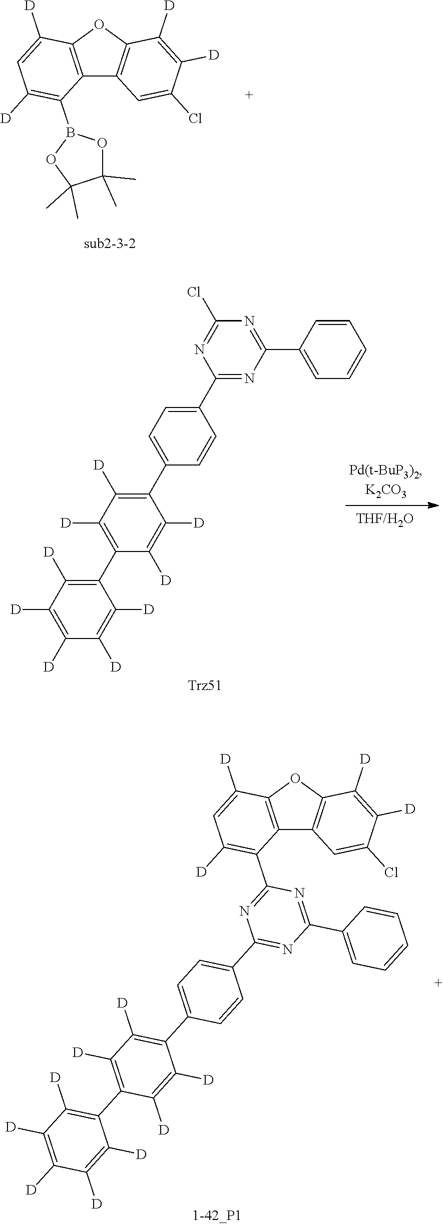 [SMILES: [2H]c1cc([2H])c2oc3c([2H])c([2H])c(Cl)cc3c2c1B1OC(C)(C)C(C)(C)O1 ; [2H]c1c([2H])c([2H])c(-c2c([2H])c([2H])c(-c3ccc(-c4nc(Cl)nc(-c5ccccc5)n4)cc3)c([2H])c2[2H])c([2H])c1[2H] ; [2H]c1cc([2H])c2oc3c([2H])c([2H])c(Cl)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

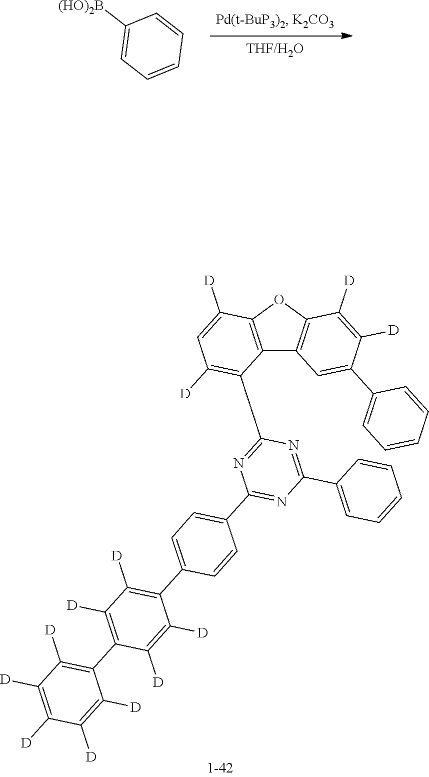 [SMILES: OB(O)c1ccccc1 ; [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1]

Sub2-3-2 (15 g, 45.1 mmol) and Trz51 (20.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-42_P1. (Yield: 75%, MS: [M+H]+=599)

Compound 1-42_P1 (15 g, 25 mmol) and phenylboronic acid (3.2 g, 26.3 mmol) were added to 300 ml of THF, and the mixture was stirred and refluxed. Then, potassium carbonate (10.4 g, 75.1 mmol) was dissolved in 31 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.9 g of Compound 1-42. (Yield: 74%, MS: [M+H]+=641)

### Synthesis Example 1-43

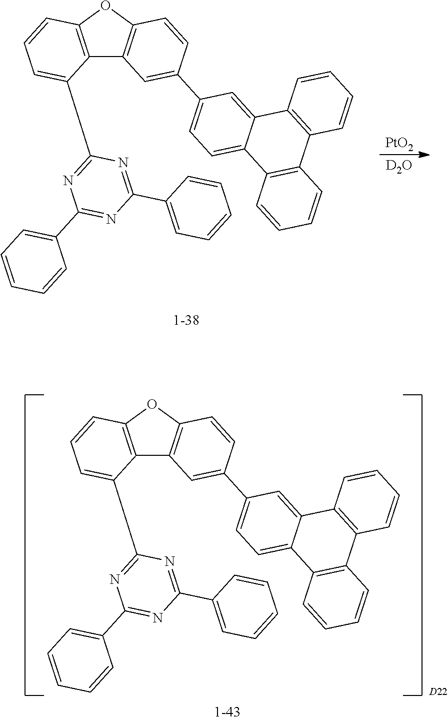

Compound 1-38 (10 g, 16 mmol), PtO₂ (1.1 g, 4.8 mmol) and D₂O (80 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 3.9 g of Compound 1-43. (Yield: 38%, MS: [M+H]+=649)

### Synthesis Example 1-44

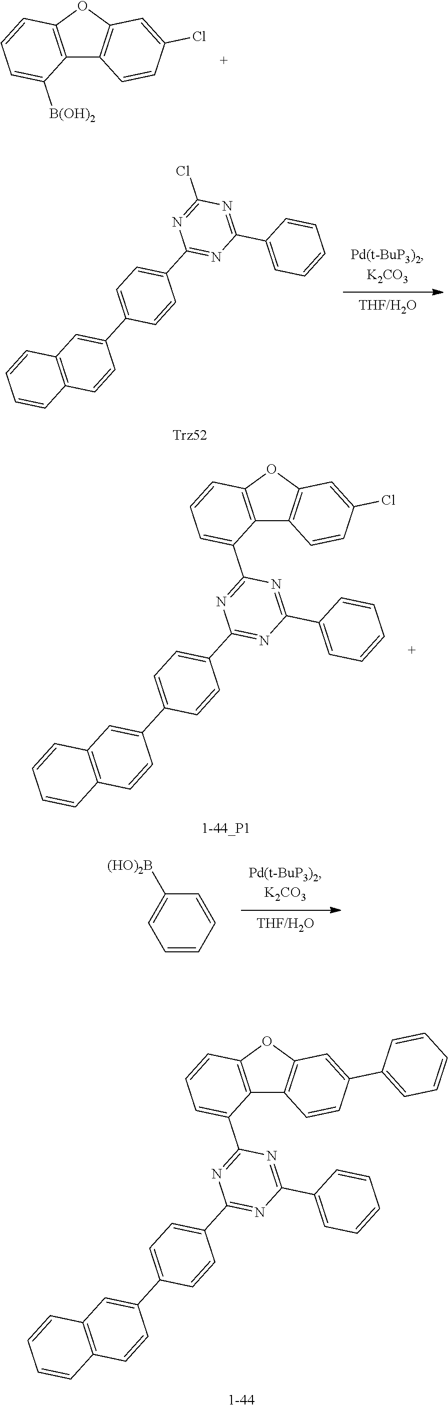 [SMILES: OB(O)c1cccc2oc3cc(Cl)ccc3c12 ; Clc1nc(-c2ccccc2)nc(-c2ccc(-c3ccc4ccccc4c3)cc2)n1 ; Clc1ccc2c(c1)oc1cccc(-c3nc(-c4ccccc4)nc(-c4ccc(-c5ccc6ccccc6c5)cc4)n3)c12 ; OB(O)c1ccccc1 ; c1ccc(-c2ccc3c(c2)oc2cccc(-c4nc(-c5ccccc5)nc(-c5ccc(-c6ccc7ccccc7c6)cc5)n4)c23)cc1]

(7-Chlorodibenzo[b,d]furan-1-yl)boronic acid (15 g, 60.9 mmol) and Trz52 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.2 g of Compound 1-44_P1. (Yield: 74%, MS: [M+H]+=560)

Compound 1-44_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.6 g of Compound 1-44. (Yield: 72%, MS: [M+H]+=602)

### Synthesis Example 1-45

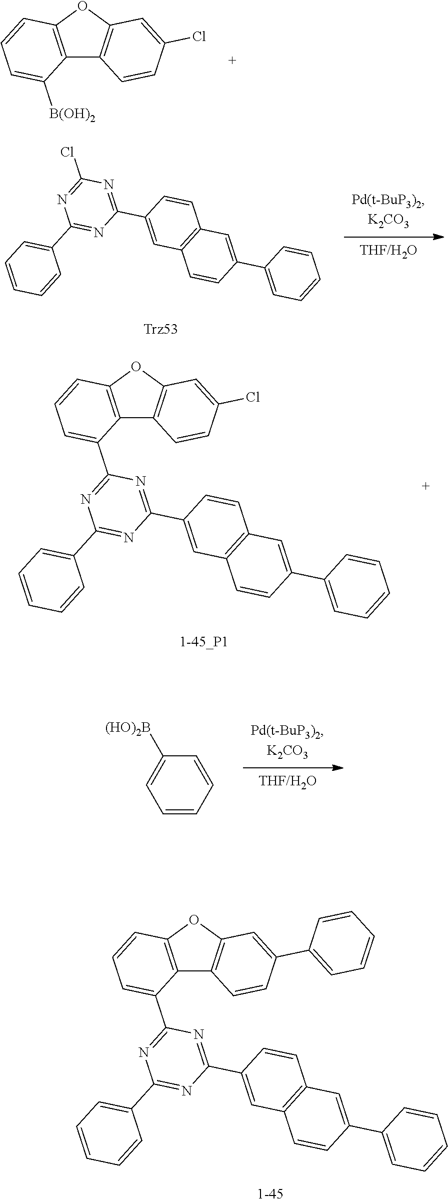

(7-Chlorodibenzo[b,d]furan-1-yl)boronic acid (15 g, 60.9 mmol) and Trz53 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.2 g of Compound 1-45_P1. (Yield: 74%, MS: [M+H]+=560)

Compound 1-45_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.1 g of Compound 1-45. (Yield: 75%, MS: [M+H]+=602)

### Synthesis Example 1-46

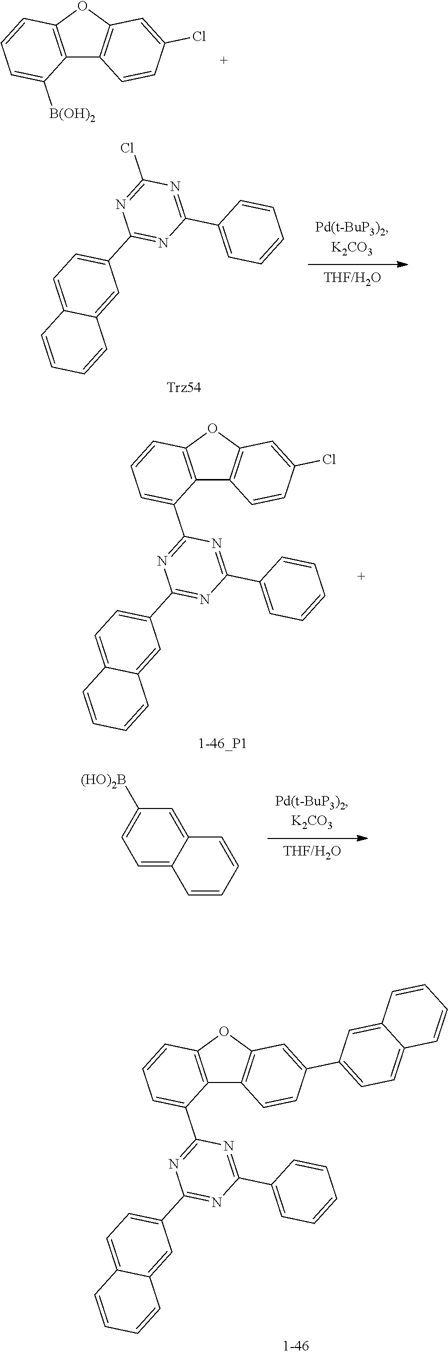 [SMILES: OB(O)c1cccc2oc3cc(Cl)ccc3c12 ; Clc1nc(-c2ccccc2)nc(-c2ccc3ccccc3c2)n1 ; Clc1ccc2c(c1)oc1cccc(-c3nc(-c4ccccc4)nc(-c4ccc5ccccc5c4)n3)c12 ; OB(O)c1ccc2ccccc2c1 ; c1ccc(-c2nc(-c3ccc4ccccc4c3)nc(-c3cccc4oc5cc(-c6ccc7ccccc7c6)ccc5c34)n2)cc1]

(7-Chlorodibenzo[b,d]furan-1-yl)boronic acid (15 g, 60.9 mmol) and Trz54 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.8 g of Compound 1-46_P1. (Yield: 74%, MS: [M+H]+=484)

Compound 1-46_P1 (15 g, 31 mmol) and naphthalen-2-ylboronic acid (5.6 g, 32.5 mmol) were added to 300 ml of THF, and the mixture was stirred and refluxed. Then, potassium carbonate (12.9 g, 93 mmol) was dissolved in 39 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.3 g of Compound 1-46. (Yield: 69%, MS: [M+H]+=576)

### Synthesis Example 1-47

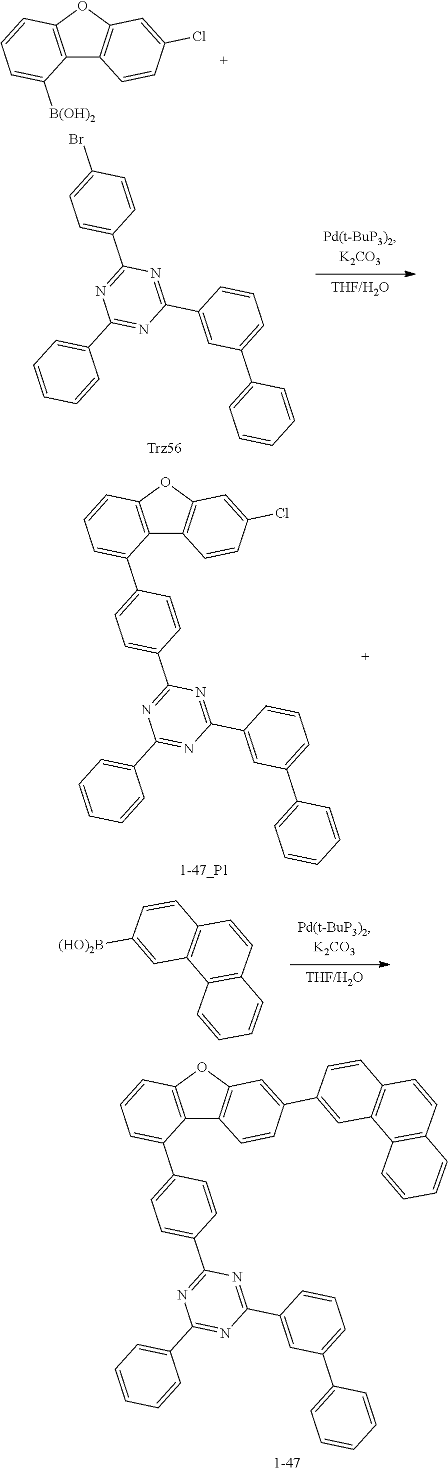

(7-Chlorodibenzo[b,d]furan-1-yl)boronic acid (15 g, 60.9 mmol) and Trz56 (29.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.9 g of Compound 1-47_P1. (Yield: 67%, MS: [M+H]+=586)

Compound 1-47_P1 (15 g, 25.6 mmol) and phenanthren-3-ylboronic acid (6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.3 g of Compound 1-47. (Yield: 66%, MS: [M+H]+=728)

### Synthesis Example 1-48

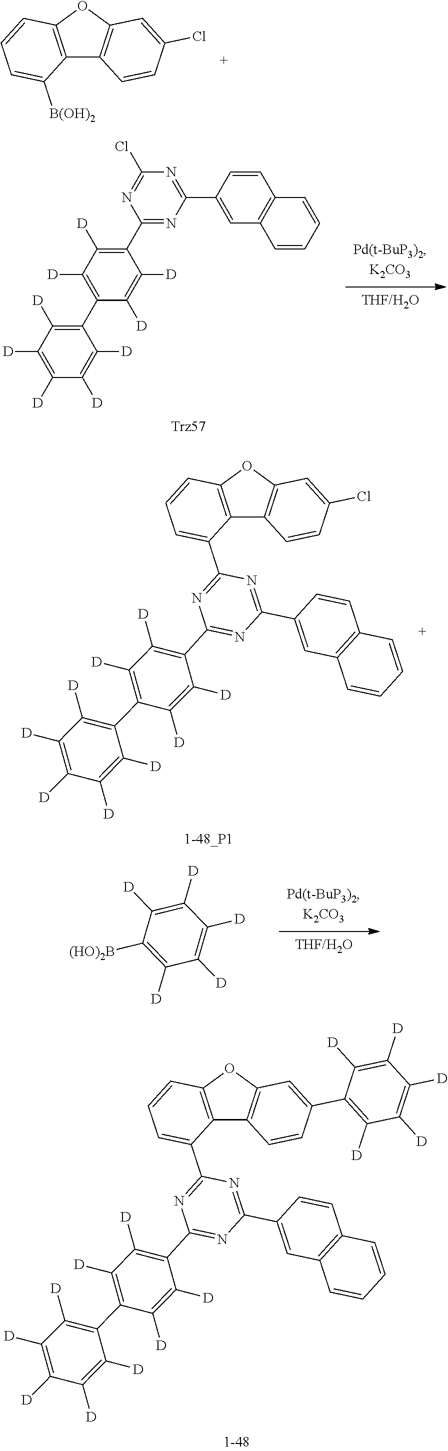 [SMILES: OB(O)c1cccc2oc3cc(Cl)ccc3c12 ; [2H]c1c([2H])c([2H])c(-c2c([2H])c([2H])c(-c3nc(Cl)nc(-c4ccc5ccccc5c4)n3)c([2H])c2[2H])c([2H])c1[2H] ; [2H]c1c([2H])c([2H])c(-c2c([2H])c([2H])c(-c3nc(-c4ccc5ccccc5c4)nc(-c4cccc5oc6cc(Cl)ccc6c45)n3)c([2H])c2[2H])c([2H])c1[2H] ; [2H]c1c([2H])c([2H])c(B(O)O)c([2H])c1[2H] ; [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H]]

(7-Chlorodibenzo[b,d]furan-1-yl)boronic acid (15 g, 60.9 mmol) and Trz57 (25.8 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.6 g of Compound 1-48_P1. (Yield: 71%, MS: [M+H]+=569)

Compound 1-48_P1 (15 g, 26.4 mmol) and (phenyl-d5) boronic acid (3.5 g, 27.7 mmol) were added to 300 ml of THF, and the mixture was stirred and refluxed. Then, potassium carbonate (10.9 g, 79.1 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.7 g of Compound 1-48. (Yield: 72%, MS: [M+H]+=616)

### Synthesis Example 1-49

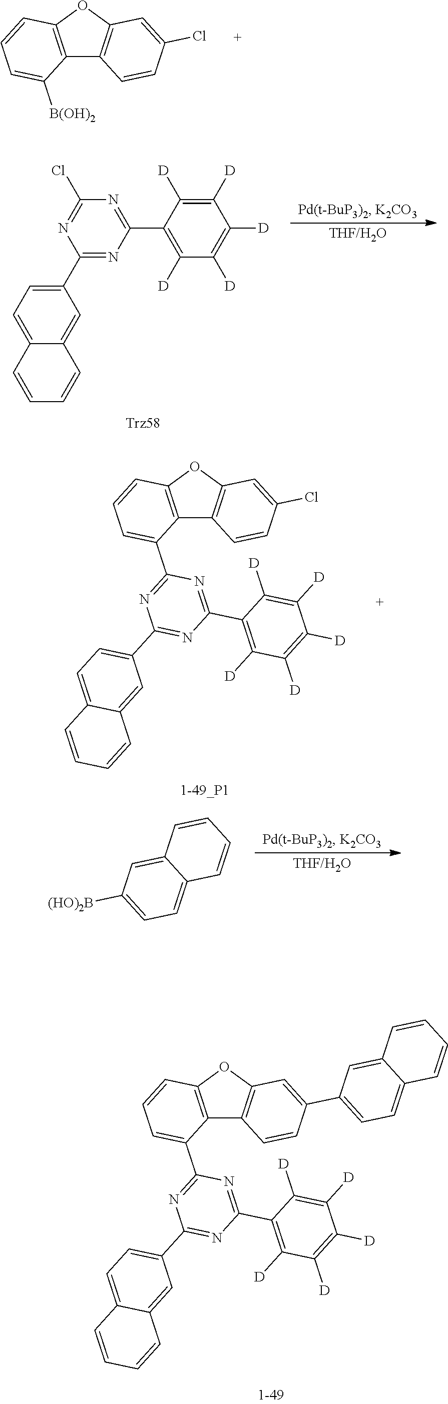

(7-Chlorodibenzo[b,d]furan-1-yl)boronic acid (15 g, 60.9 mmol) and Trz58 (20.6 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.2 g of Compound 1-49_P1. (Yield: 68%, MS: [M+H]+=489)

Compound 1-49_P1 (15 g, 30.7 mmol) and naphthalen-2-ylboronic acid (5.5 g, 32.2 mmol) were added to 300 ml of THF, and the mixture was stirred and refluxed. Then, potassium carbonate (12.7 g, 92 mmol) was dissolved in 38 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.6 g of Compound 1-49. (Yield: 71%, MS: [M+H]+=581)

### Synthesis Example 1-50

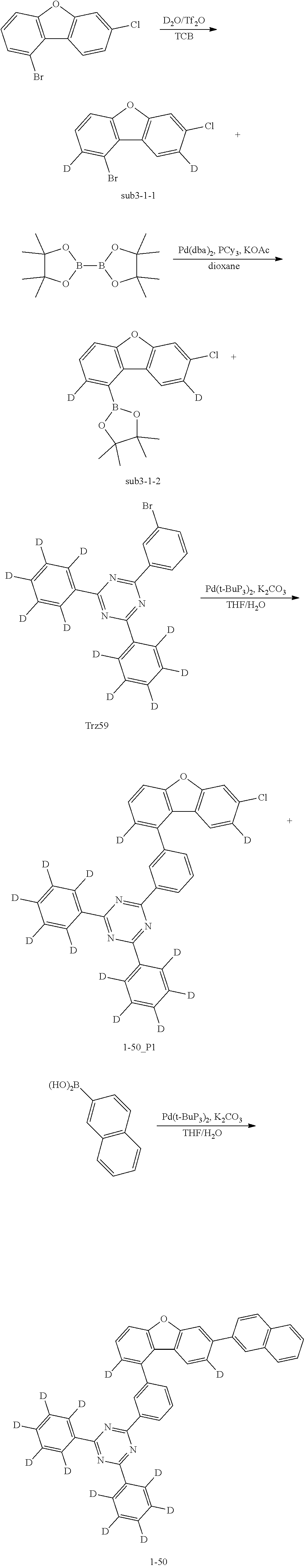

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of Compound sub3-1-1. (Yield: 40%, MS: [M+H]+=283)

Sub3-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 4 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.4 g of sub3-1-2. (Yield: 65%, MS: [M+H]+=331)

Sub3-1-2 (15 g, 45.4 mmol) and Trz59 (19 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.3 g of Compound 1-50_P1. (Yield: 73%, MS: [M+H]+=522)

Compound 1-50_P1 (15 g, 28.7 mmol) and naphthalen-2-ylboronic acid (5.2 g, 30.2 mmol) were added to 300 ml of THF, and the mixture was stirred and refluxed. Then, potassium carbonate (11.9 g, 86.2 mmol) was dissolved in 36 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.5 g of Compound 1-50. (Yield: 71%, MS: [M+H]+=614)

### Synthesis Example 1-51

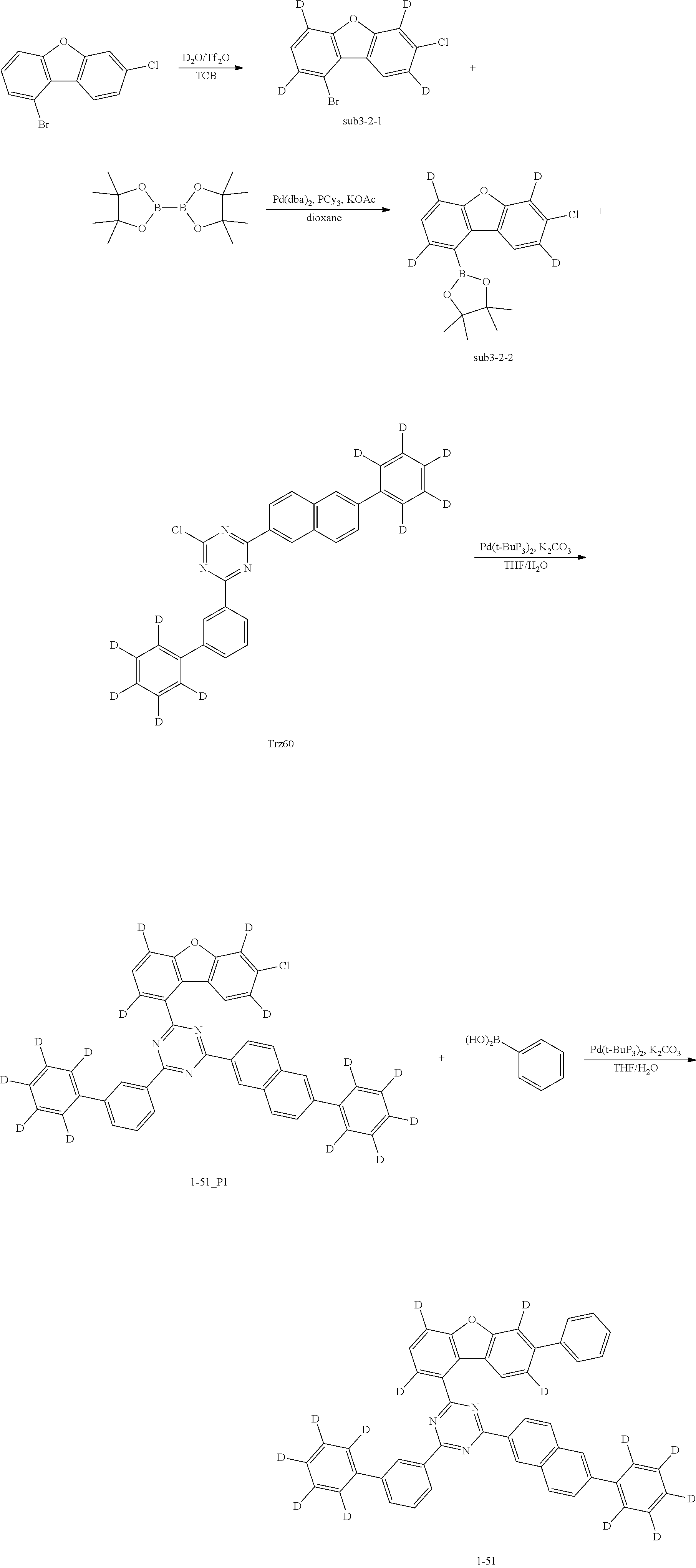

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-7-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-7-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.7 g of sub3-2-1. (Yield: 44%, MS: [M+H]+=285)

Sub3-2-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 11.7 g of sub3-2-2. (Yield: 67%, MS: [M+H]+=333)

Sub3-2-2 (15 g, 45.1 mmol) and Trz60 (22.7 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.9 g of Compound 1-51_P1. (Yield: 68%, MS: [M+H]+=650)

Compound 1-51_P1 (15 g, 23.1 mmol) and phenylboronic acid (3 g, 24.2 mmol) were added to 300 ml of THF, and the mixture was stirred and refluxed. Then, potassium carbonate (9.6 g, 69.2 mmol) was dissolved in 29 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.6 g of Compound 1-51. (Yield: 73%, MS: [M+H]+=692)

### Synthesis Example 1-52

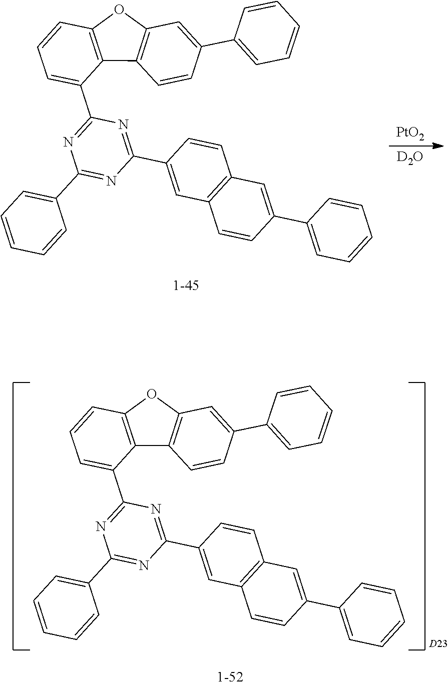

Compound 1-45 (10 g, 16.6 mmol), PtO₂ (1.1 g, 5 mmol) and D₂O (83 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 3.1 g of Compound 1-52. (Yield: 30%, MS: [M+H]+=626)

### Synthesis Example 1-53

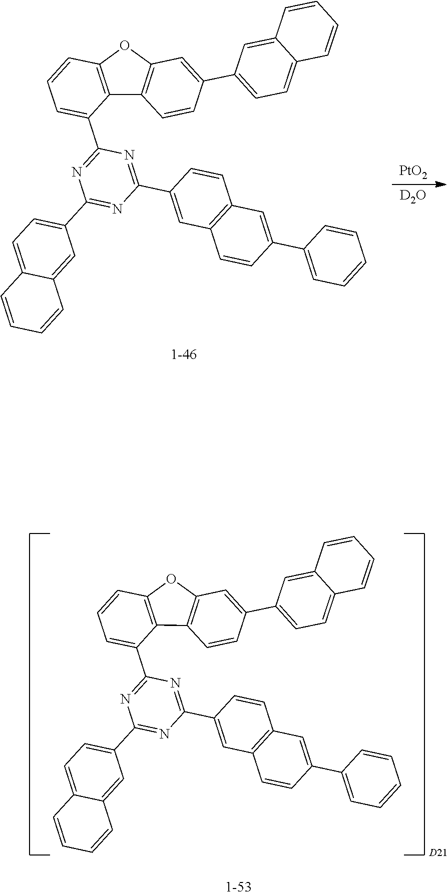

Compound 1-46 (10 g, 17.4 mmol), PtO₂ (1.2 g, 5.2 mmol) and D₂O (87 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 3.9 g of Compound 1-53. (Yield: 38%, MS: [M+H]+=598)

### Synthesis Example 1-54

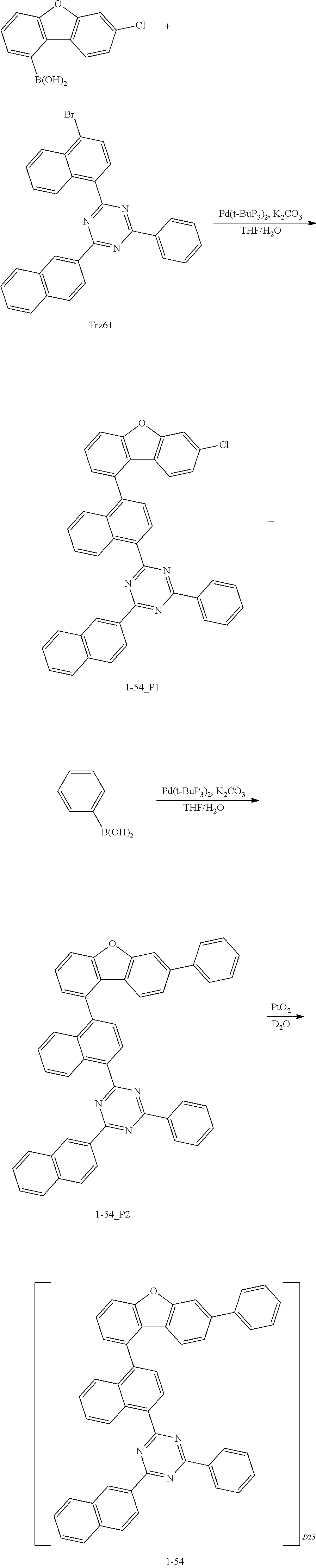

(7-Chlorodibenzo[b,d]furan-1-yl)boronic acid (15 g, 60.9 mmol) and Trz61 (31.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.5 g of Compound 1-54_P1. (Yield: 66%, MS: [M+H]+=610)

Compound 1-54_P1 (15 g, 24.6 mmol) and phenylboronic acid (3.1 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.6 g of Compound 1-54_P2. (Yield: 66%, MS: [M+H]+=652)

Compound 1-54_P2 (10 g, 15.3 mmol), PtO₂ (1 g, 4.6 mmol) and D₂O (77 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 4.6 g of Compound 1-54. (Yield: 44%, MS: [M+H]+=678)

### Synthesis Example 1-55

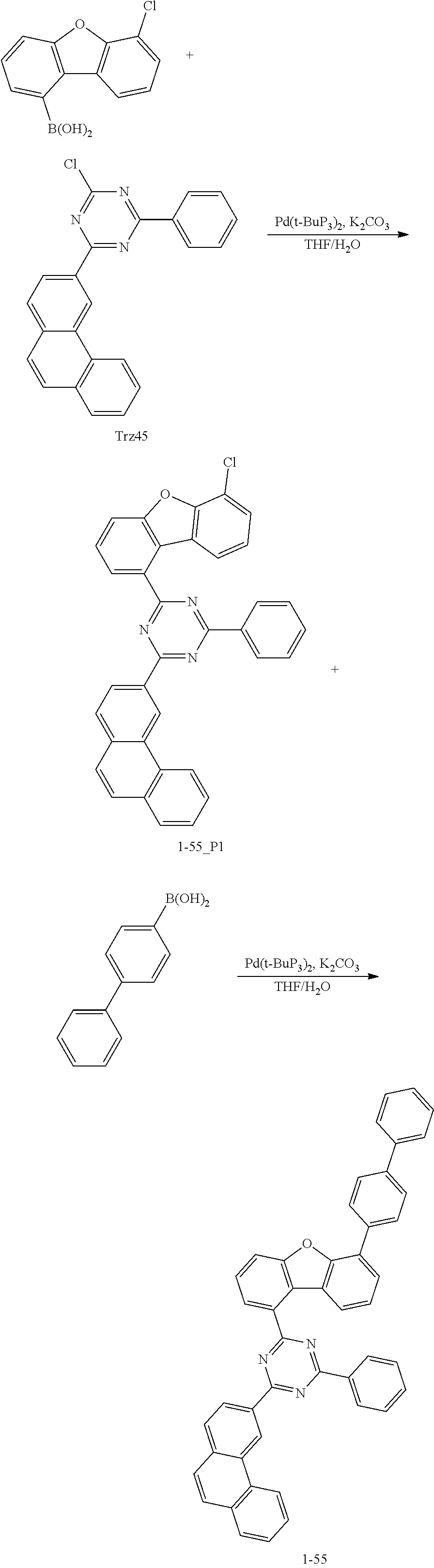

(6-Chlorodibenzo[b,d]furan-1-yl)boronic acid (15 g, 60.9 mmol) and Trz45 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-55_P1. (Yield: 72%, MS: [M+H]+=534)

Compound 1-55_P1 (15 g, 28.1 mmol) and [1,1′-biphenyl]-4-ylboronic acid (5.8 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.4 g of Compound 1-55. (Yield: 73%, MS: [M+H]+=652)

### Synthesis Example 1-56

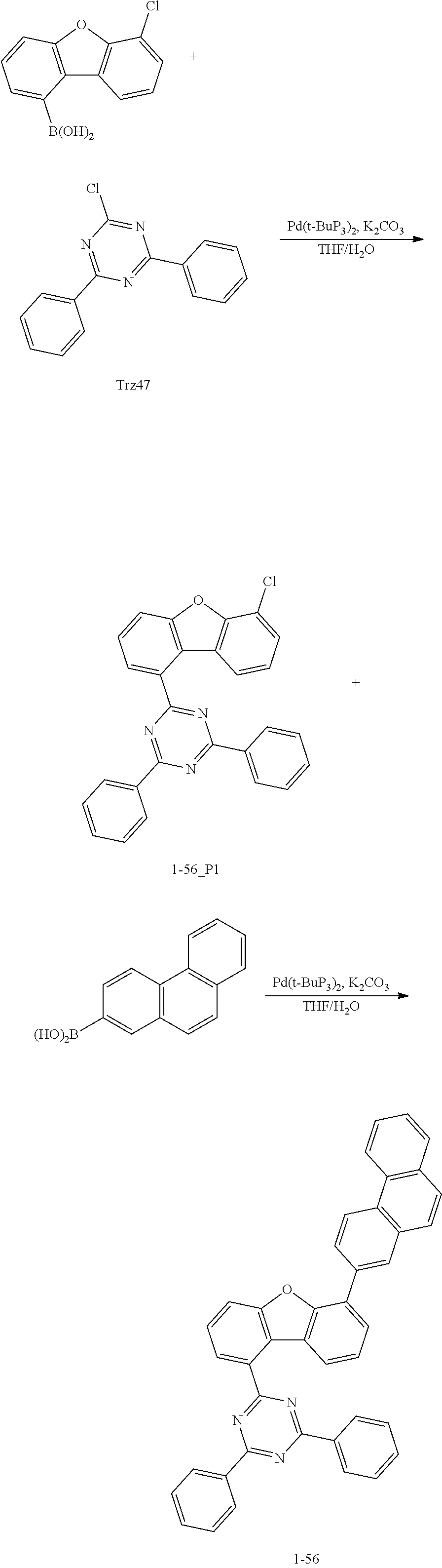

(6-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.4 g of Compound 1-56_P1. (Yield: 66%, MS: [M+H]+=434)

Compound 1-56_P1 (15 g, 34.6 mmol) and phenanthren-2-ylboronic acid (8.1 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-56. (Yield: 67%, MS: [M+H]+=576)

### Synthesis Example 1-57

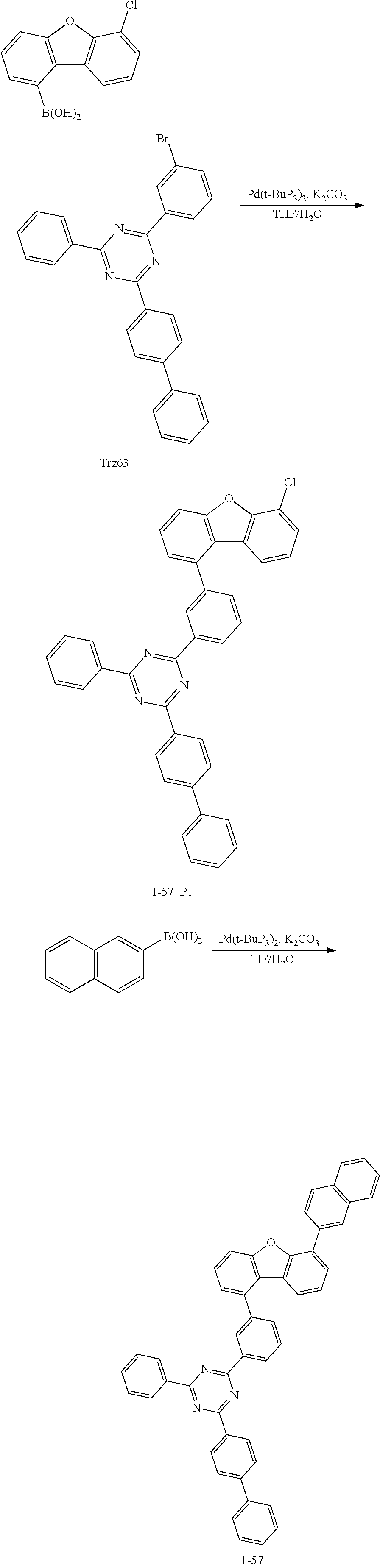

(6-Chlorodibenzo[b,d]furan-1-yl)boronic acid (15 g, 60.9 mmol) and Trz63 (29.7 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.9 g of Compound 1-57_P1. (Yield: 67%, MS: [M+H]+=586)

Compound 1-57_P1 (15 g, 25.6 mmol) and naphthalen-2-ylboronic acid (4.6 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.8 g of Compound 1-57. (Yield: 74%, MS: [M+H]+=678)

### Synthesis Example 1-58

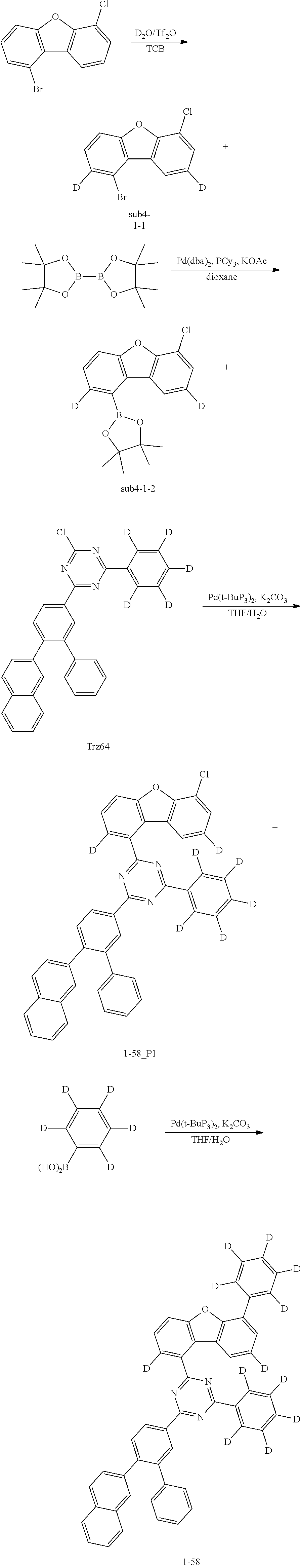

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-6-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-6-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.8 g of sub4-1-1. (Yield: 45%, MS: [M+H]+=283)

Sub4-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 13.1 g of sub4-1-2. (Yield: 75%, MS: [M+H]+=331)

Sub4-1-2 (15 g, 45.4 mmol) and Trz64 (22.6 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.4 g of Compound 1-58_P1. (Yield: 70%, MS: [M+H]+=643)

Compound 1-58_P1 (15 g, 23.3 mmol) and (phenyl-d5) boronic acid (3.1 g, 24.5 mmol) were added to 300 ml of THF, and the mixture was stirred and refluxed. Then, potassium carbonate (9.7 g, 70 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.7 g of Compound 1-58. (Yield: 73%, MS: [M+H]+=690)

### Synthesis Example 1-59

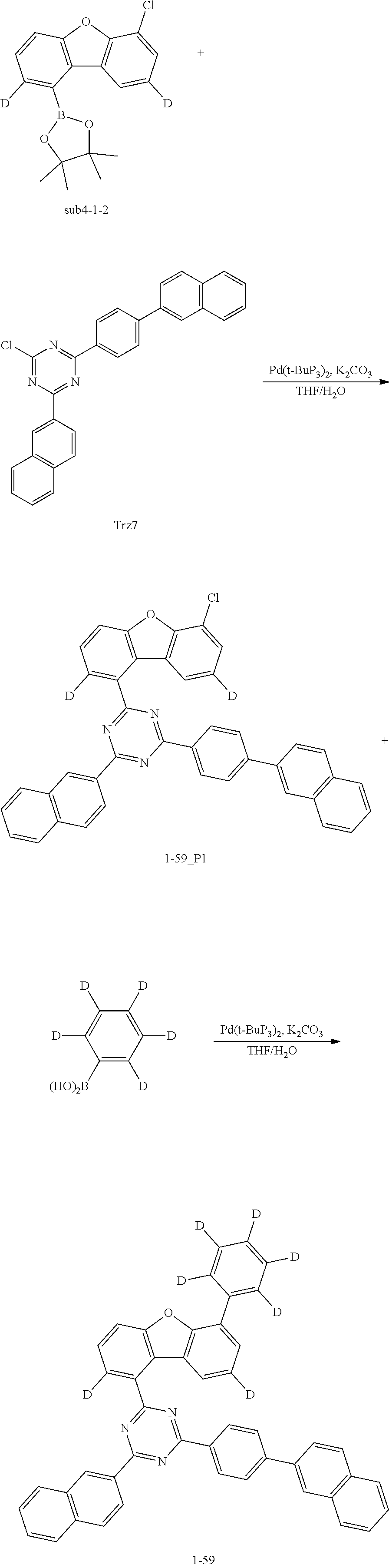

Sub4-1-2 (15 g, 45.4 mmol) and Trz7 (21.1 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18 g of Compound 1-59_P1. (Yield: 65%, MS: [M+H]+=612)

Compound 1-59_P1 (15 g, 24.5 mmol) and (phenyl-d5) boronic acid (3.3 g, 25.7 mmol) were added to 300 ml of THF, and the mixture was stirred and refluxed. Then, potassium carbonate (10.2 g, 73.5 mmol) was dissolved in 30 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.1 g of Compound 1-59. (Yield: 69%, MS: [M+H]+=659)

### Synthesis Example 1-60

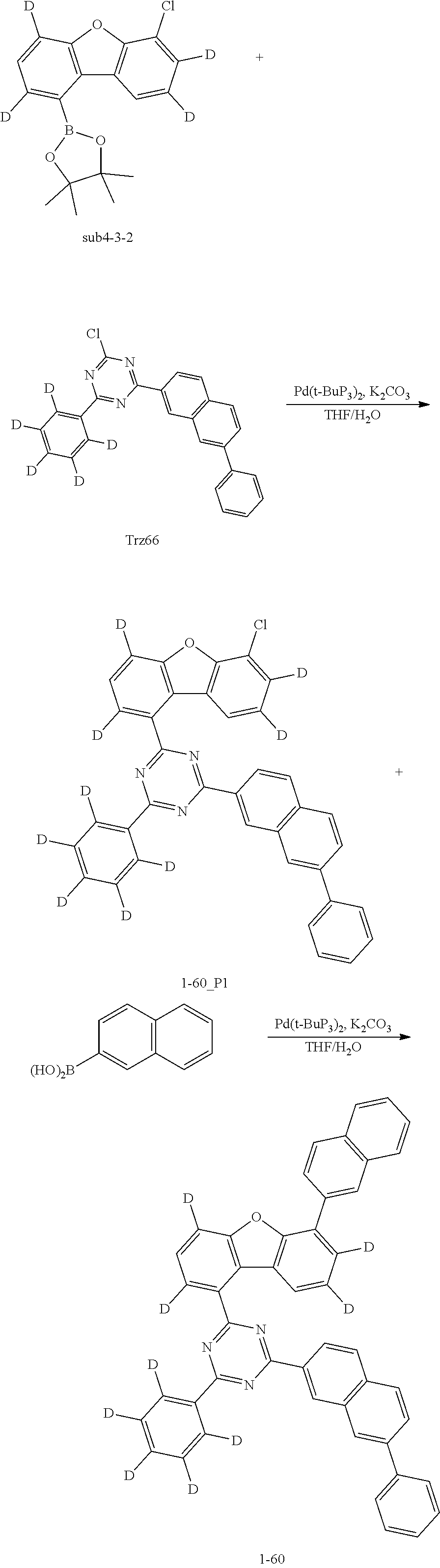

Sub4-3-2 (15 g, 45.1 mmol) and Trz66 (18.9 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19 g of Compound 1-60_P1. (Yield: 74%, MS: [M+H]+=569)

Compound 1-60_P1 (15 g, 26.4 mmol) and naphthalen-2-ylboronic acid (4.8 g, 27.7 mmol) were added to 300 ml of THF, and the mixture was stirred and refluxed. Then, potassium carbonate (10.9 g, 79.1 mmol) was dissolved in 33 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.5 g of Compound 1-60. (Yield: 72%, MS: [M+H]+=661)

### Synthesis Example 1-61

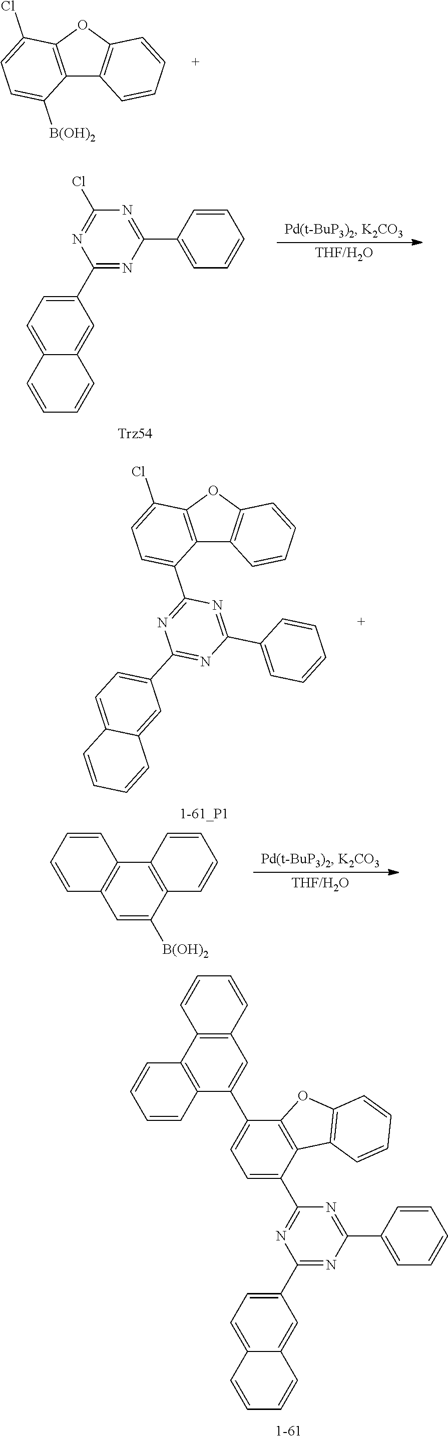

(4-Chlorodibenzo[b,d]furan-1-yl)boronic acid (15 g, 60.9 mmol) and Trz54 (20.3 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.1 g of Compound 1-61_P1. (Yield: 65%, MS: [M+H]+=484)

Compound 1-61_P1 (15 g, 31 mmol) and phenanthren-9-ylboronic acid (7.2 g, 32.6 mmol) were added to 300 ml of THF, and the mixture was stirred and refluxed. Then, potassium carbonate (12.9 g, 93.1 mmol) was dissolved in 39 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.2 g of Compound 1-61. (Yield: 73%, MS: [M+H]+=626)

### Synthesis Example 1-62

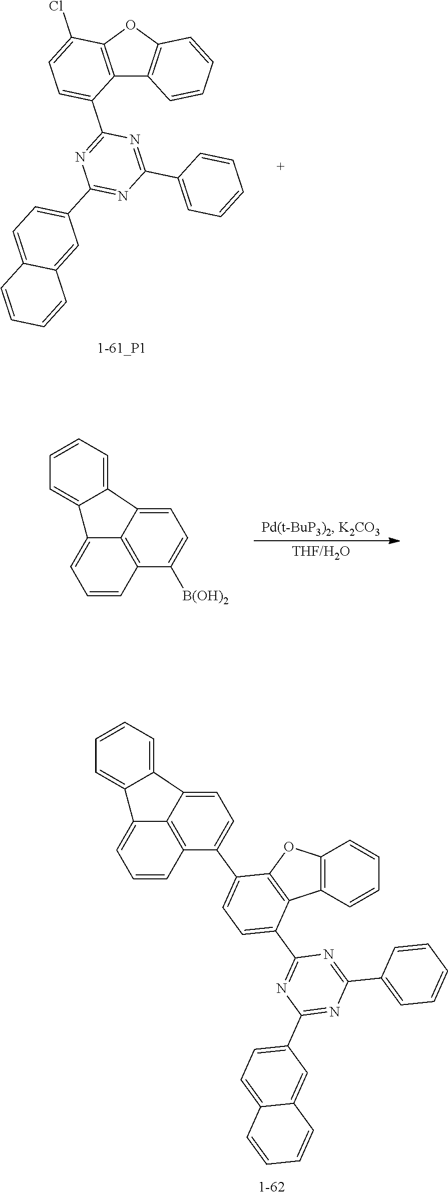

Compound 1-61_P1 (15 g, 31 mmol) and fluoranthen-3-ylboronic acid (8 g, 32.6 mmol) were added to 300 ml of THF, and the mixture was stirred and refluxed. Then, potassium carbonate (12.9 g, 93.1 mmol) was dissolved in 39 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 13.3 g of Compound 1-62. (Yield: 66%, MS: [M+H]+=650)

### Synthesis Example 1-63

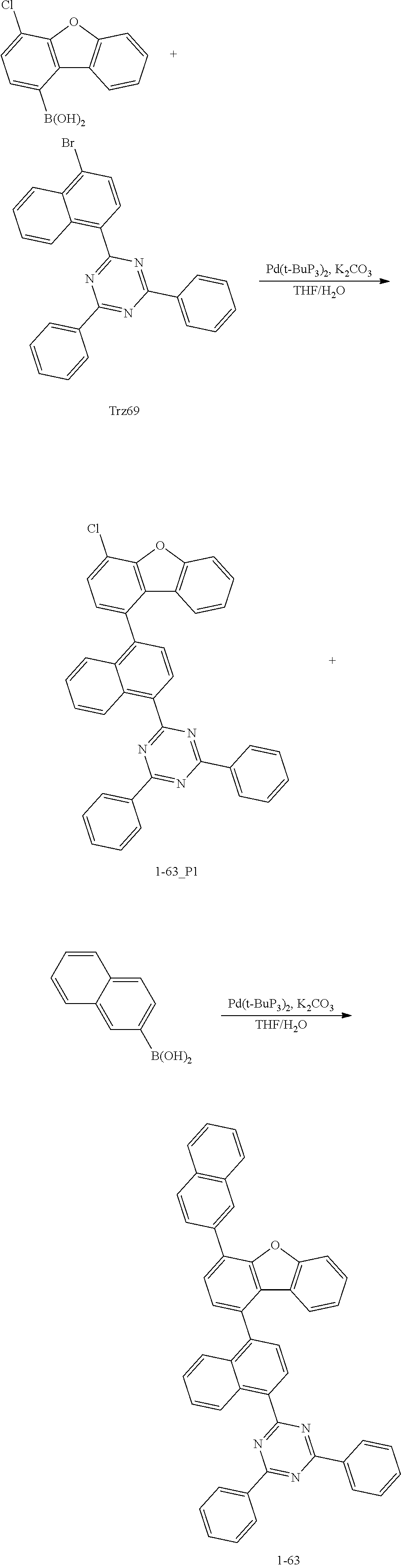

(4-Chlorodibenzo[b,d]furan-1-yl)boronic acid (15 g, 60.9 mmol) and Trz69 (28 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.2 g of Compound 1-63_P1. (Yield: 71%, MS: [M+H]+=560)

Compound 1-63_P1 (15 g, 26.8 mmol) and naphthalen-2-ylboronic acid (4.8 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.5 g of Compound 1-63. (Yield: 66%, MS: [M+H]+=652)

### Synthesis Example 1-64

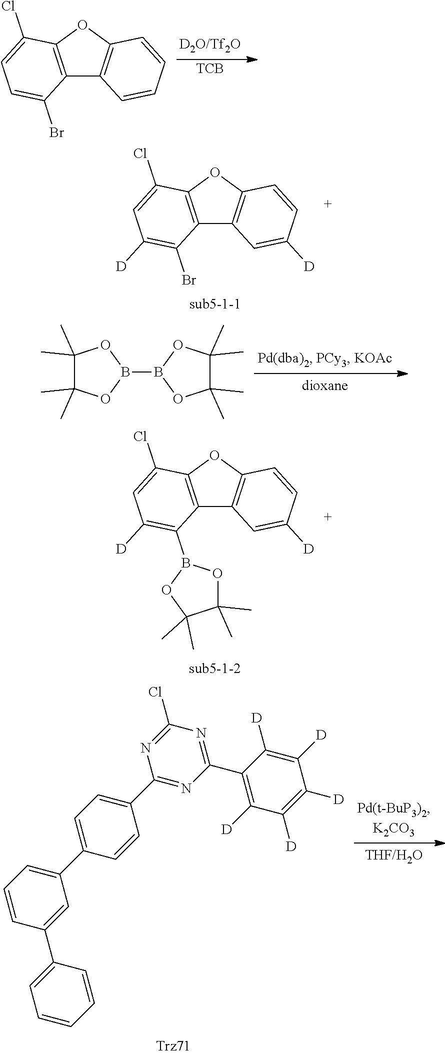

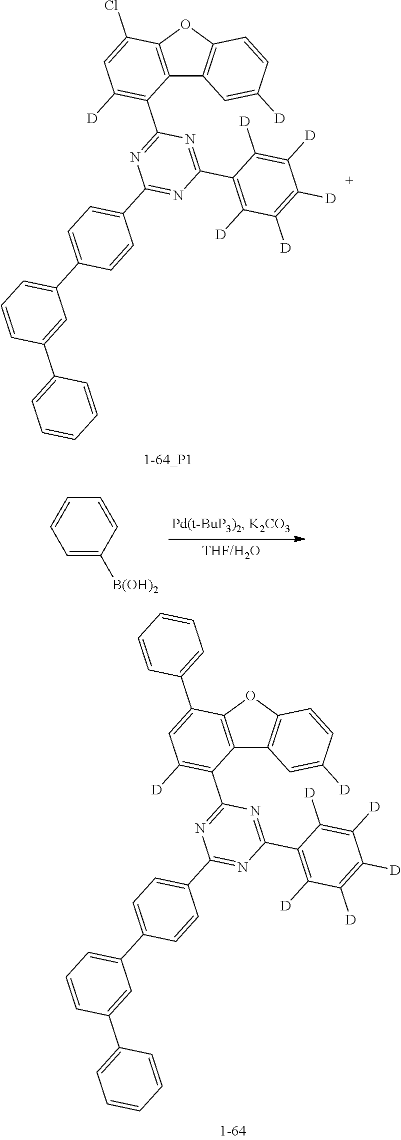 [SMILES: [2H]c1ccc2oc3c(Cl)cc([2H])c(-c4nc(-c5ccc(-c6cccc(-c7ccccc7)c6)cc5)nc(-c5c([2H])c([2H])c([2H])c([2H])c5[2H])n4)c3c2c1 ; OB(O)c1ccccc1 ; [2H]c1ccc2oc3c(-c4ccccc4)cc([2H])c(-c4nc(-c5ccc(-c6cccc(-c7ccccc7)c6)cc5)nc(-c5c([2H])c([2H])c([2H])c([2H])c5[2H])n4)c3c2c1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating to 140° C. and then keeping that temperature. After the reaction for 3 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.5 g of sub5-1-1. (Yield: 43%, MS: [M+H]+=283)

Sub5-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 10.8 g of sub5-1-2. (Yield: 62%, MS: [M+H]+=331)

Sub5-1-2 (15 g, 45.4 mmol) and Trz71 (20.2 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19.9 g of Compound 1-64_P1. (Yield: 74%, MS: [M+H]+=594)

Compound 1-64_P1 (15 g, 25.3 mmol) and phenylboronic acid (3.2 g, 26.6 mmol) were added to 300 ml of THF, and the mixture was stirred and refluxed. Then, potassium carbonate (10.5 g, 75.9 mmol) was dissolved in 31 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.7 g of Compound 1-64. (Yield: 73%, MS: [M+H]+=635)

### Synthesis Example 1-65

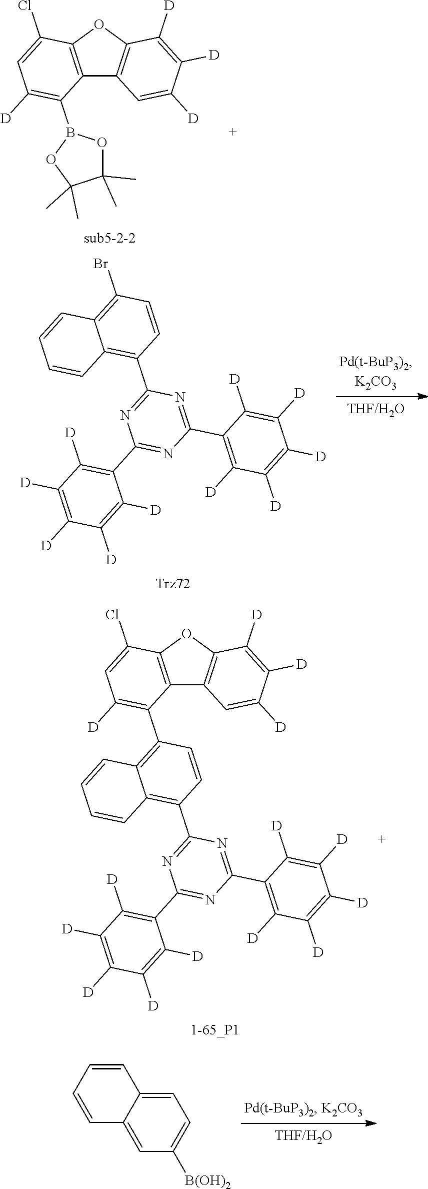 [SMILES: [2H]c1cc2c(oc3c(Cl)cc([2H])c(B4OC(C)(C)C(C)(C)O4)c32)c([2H])c1[2H] ; [2H]c1c([2H])c([2H])c(-c2nc(-c3c([2H])c([2H])c([2H])c([2H])c3[2H])nc(-c3ccc(Br)c4ccccc34)n2)c([2H])c1[2H] ; [2H]c1cc2c(oc3c(Cl)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H] ; OB(O)c1ccc2ccccc2c1]

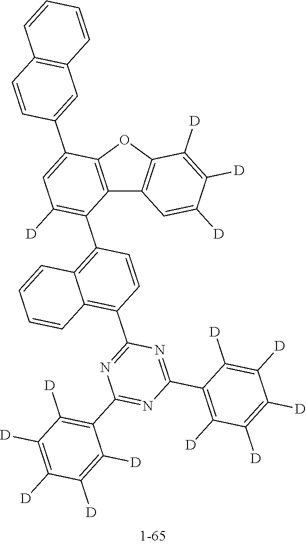 [SMILES: [2H]c1cc2c(oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H]]

Sub5-2-2 (15 g, 45.1 mmol) and Trz72 (21.2 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.4 g of Compound 1-65_P1. (Yield: 71%, MS: [M+H]+=574)

Compound 1-65_P1 (15 g, 26.1 mmol) and naphthalen-2-ylboronic acid (4.7 g, 27.4 mmol) were added to 300 ml of THF, and the mixture was stirred and refluxed. Then, potassium carbonate (10.8 g, 78.4 mmol) was dissolved in 32 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.6 g of Compound 1-65. (Yield: 67%, MS: [M+H]+=666)

### Synthesis Example 1-66

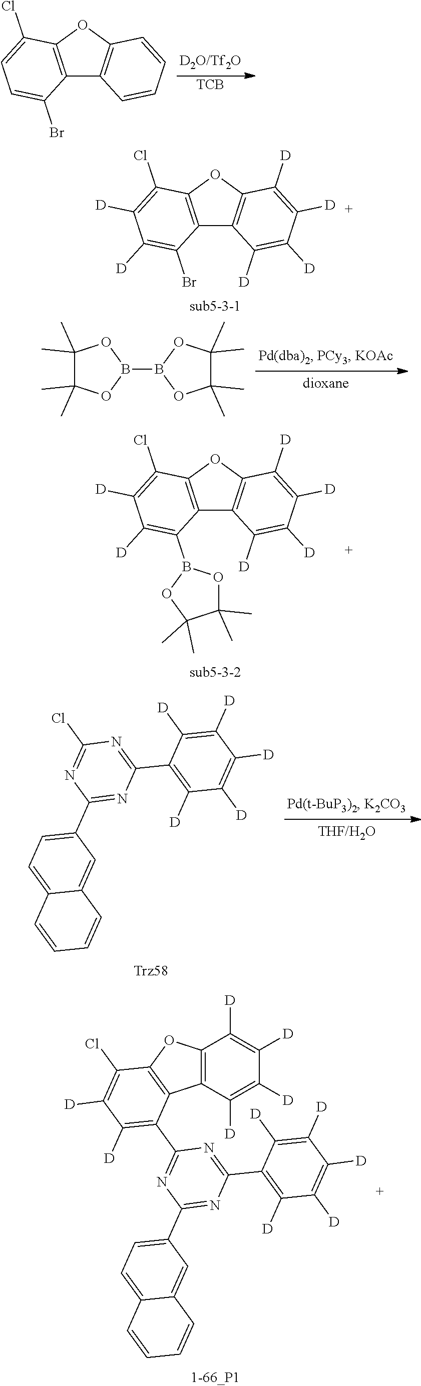

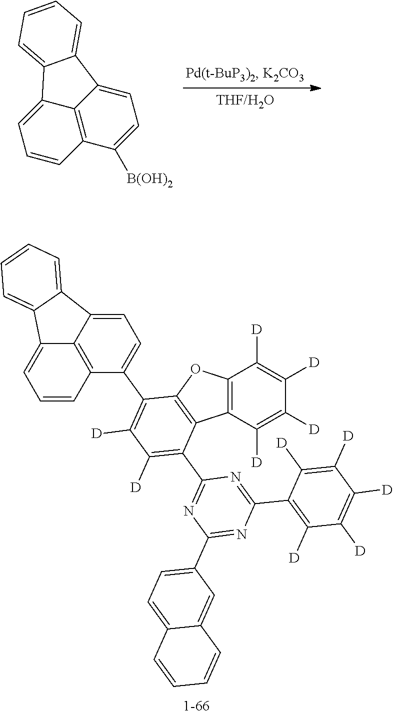 [SMILES: OB(O)c1ccc2c3c(cccc13)-c1ccccc1-2 ; [2H]c1c([2H])c([2H])c(-c2nc(-c3ccc4ccccc4c3)nc(-c3c([2H])c([2H])c(-c4ccc5c6c(cccc46)-c4ccccc4-5)c4oc5c([2H])c([2H])c([2H])c([2H])c5c34)n2)c([2H])c1[2H]]

Trifluoromethanesulfonic anhydride (90.2 g, 319.7 mmol) and deuterium oxide (32 g, 1598.4 mmol) were added at 0° C. and stirred for 5 hours to prepare a solution. 1-Bromo-4-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-4-chlorodibenzo[b,d]furan and 1,2,4-trichlorobenzene, and the mixture was stirred while heating to 140° C. and then keeping that temperature. After the reaction for 18 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.8 g of sub5-3-1. (Yield: 38%, MS: [M+H]+=287)

Sub5-3-1 (15 g, 52.2 mmol) and bis(pinacolato)diboron (14.6 g, 57.4 mmol) were added to 300 ml of 1,4-dioxane, and the mixture was stirred under reflux. Then, potassium acetate (7.7 g, 78.2 mmol) was added thereto, sufficiently stirred, and then bis(dibenzylideneacetone)palladium(0) (0.9 g, 1.6 mmol) and tricyclohexylphosphine (0.9 g, 3.1 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12.9 g of sub5-3-2. (Yield: 74%, MS: [M+H]+=335)

Sub5-3-2 (15 g, 44.8 mmol) and Trz58 (15.7 g, 47.1 mmol) were added to 300 ml of THF, and the mixture was stirred and refluxed. Then, potassium carbonate (18.6 g, 134.5 mmol) was dissolved in 56 ml of water and added thereto, and the mixture was sufficiently stirred and then bis(tri-tert-butylphosphine)palladium(0) (0.2 g, 0.4 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 16.2 g of Compound 1-66_P1. (Yield: 73%, MS: [M+H]+=495)

Compound 1-66_P1 (15 g, 30.3 mmol) and fluoranthen-3-ylboronic acid (7.8 g, 31.8 mmol) were added to 300 ml of THF, and the mixture was stirred and refluxed. Then, potassium carbonate (12.6 g, 90.9 mmol) was dissolved in 38 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 15 g of Compound 1-66. (Yield: 75%, MS: [M+H]+=661)

### Synthesis Example 1-67

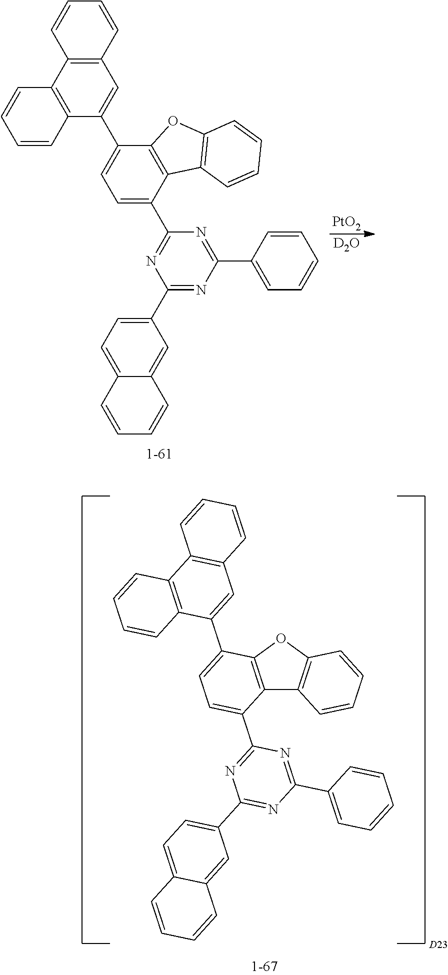

Compound 1-61 (10 g, 16 mmol), PtO₂ (1.1 g, 4.8 mmol) and D₂O (80 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO₄ and concentrated, and then the sample was purified by silica gel column chromatography to prepare 3.9 g of Compound 1-67. (Yield: 38%, MS: [M+H]+=650)

### Synthesis Example 1-68

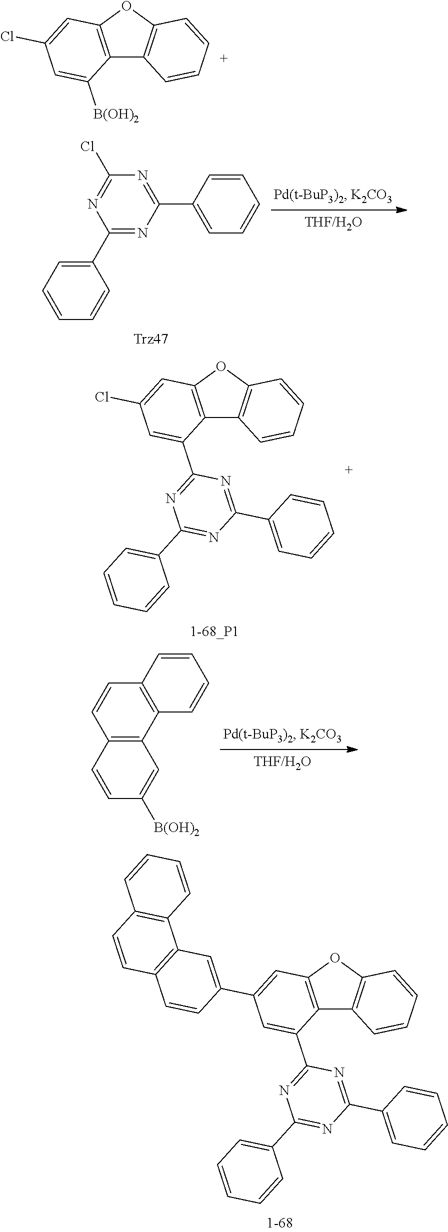

(3-Chlorodibenzo[b,d]furan-1-yl)boronic acid (15 g, 60.9 mmol) and Trz47 (17.1 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 19 g of Compound 1-68_P1. (Yield: 72%, MS: [M+H]+=434)

Compound 1-68_P1 (15 g, 34.6 mmol) and phenanthren-3-ylboronic acid (8.1 g, 36.3 mmol) were added to 300 ml of THF, and the mixture was stirred and refluxed. Then, potassium carbonate (14.3 g, 103.7 mmol) was dissolved in 43 ml of water and added thereto, and the mixture was sufficiently stirred and then bis(tri-tert-butylphosphine)palladium(0) (0.2 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 14.1 g of Compound 1-68. (Yield: 71%, MS: [M+H]+=576)

### Synthesis Example 1-69

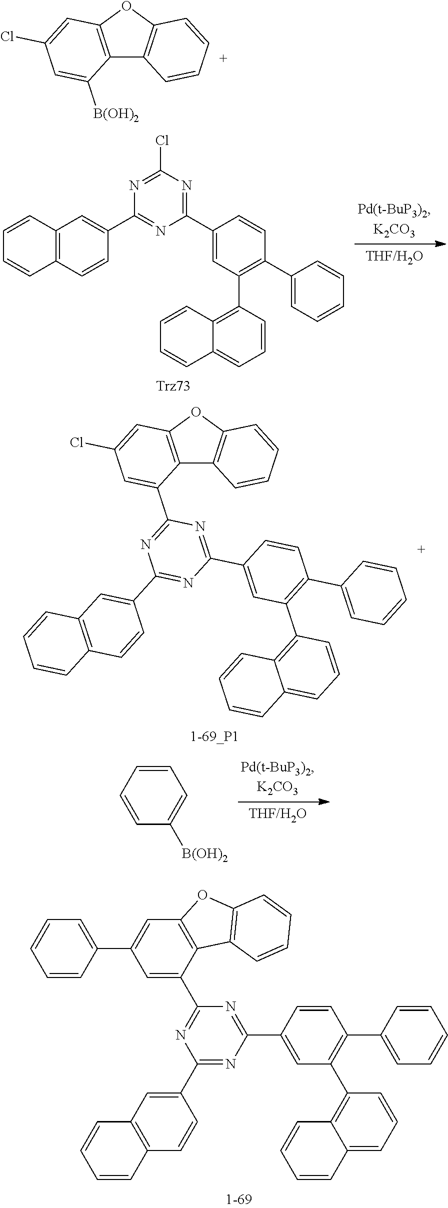

(3-Chlorodibenzo[b,d]furan-1-yl)boronic acid (15 g, 60.9 mmol) and Trz73 (33.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.6 g of Compound 1-69_P1. (Yield: 71%, MS: [M+H]+=686)

Compound 1-69_P1 (15 g, 21.9 mmol) and phenylboronic acid (2.8 g, 23 mmol) were added to 300 ml of THF, and the mixture was stirred and refluxed. Then, potassium carbonate (9.1 g, 65.6 mmol) was dissolved in 27 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.3 g of Compound 1-69. (Yield: 68%, MS: [M+H]+=758)

### Synthesis Example 1-70

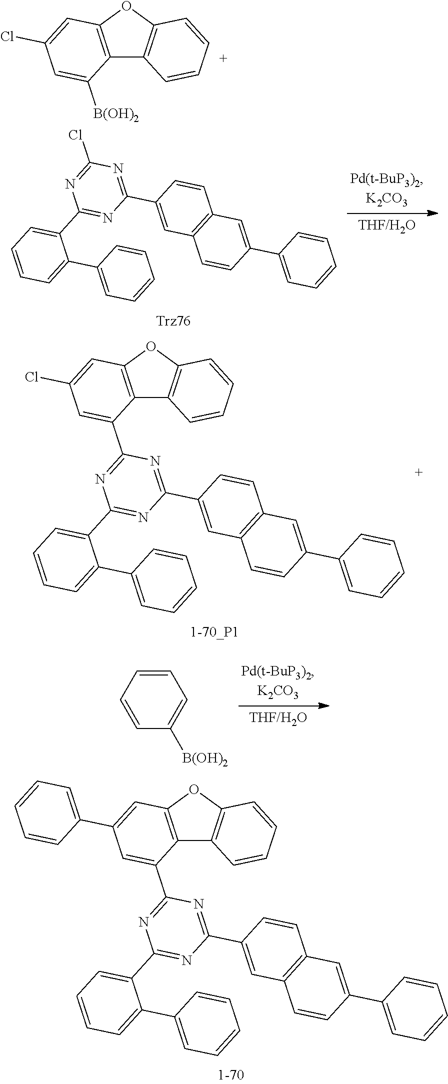

(3-Chlorodibenzo[b,d]furan-1-yl)boronic acid (15 g, 60.9 mmol) and Trz76 (30 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.5 g of Compound 1-70_P1. (Yield: 66%, MS: [M+H]+=636)

Compound 1-70_P1 (15 g, 23.6 mmol) and phenylboronic acid (3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.7 g of Compound 1-70. (Yield: 67%, MS: [M+H]+=678)

### Synthesis Example 1-71

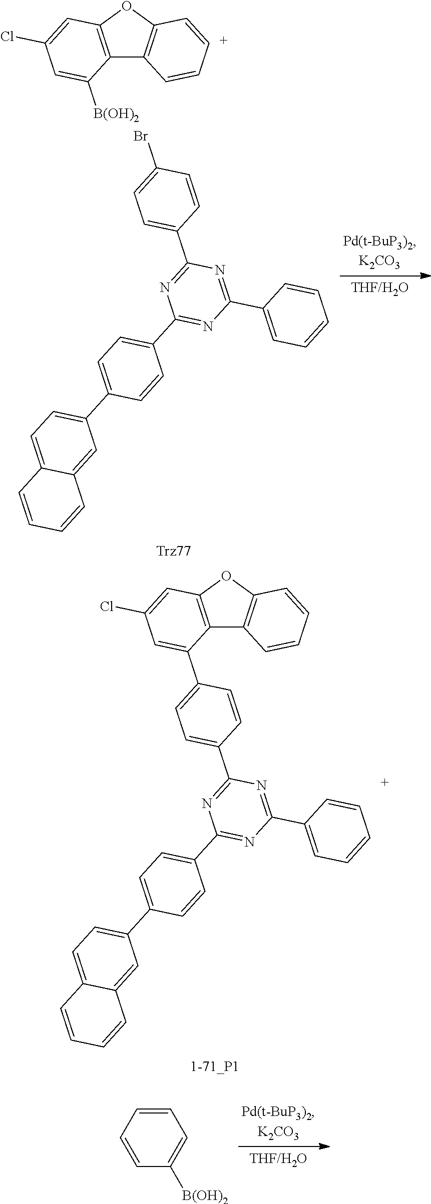 [SMILES: OB(O)c1cc(Cl)cc2oc3c(c12)C=C[C+]=C3 ; Brc1ccc(-c2nc(-c3ccccc3)nc(-c3ccc(-c4ccc5ccccc5c4)cc3)n2)cc1 ; Clc1cc(-c2ccc(-c3nc(-c4ccccc4)nc(-c4ccc(-c5ccc6ccccc6c5)cc4)n3)cc2)c2c(c1)oc1ccccc12 ; OB(O)c1ccccc1]

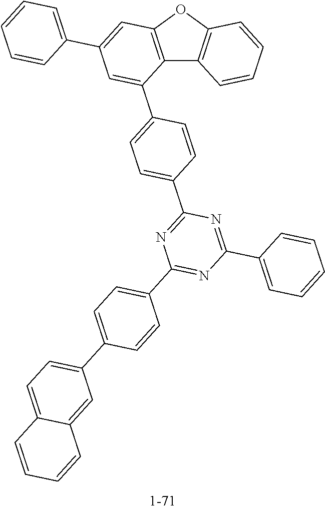 [SMILES: c1ccc(-c2cc(-c3ccc(-c4nc(-c5ccccc5)nc(-c5ccc(-c6ccc7ccccc7c6)cc5)n4)cc3)c3c(c2)oc2ccccc23)cc1]

(3-Chlorodibenzo[b,d]furan-1-yl)boronic acid (15 g, 60.9 mmol) and Trz77 (32.9 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.3 g of Compound 1-71_P1. (Yield: 68%, MS: [M+H]+=636)

Compound 1-71_P1 (15 g, 23.6 mmol) and phenylboronic acid (3 g, 24.8 mmol) were added to 300 ml of THF, and the mixture was stirred and refluxed. Then, potassium carbonate (9.8 g, 70.7 mmol) was dissolved in 29 ml of water and added thereto, and the mixture was sufficiently stirred and then bis(tri-tert-butylphosphine)palladium(0) (0.1 g, 0.2 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.5 g of Compound 1-71. (Yield: 66%, MS: [M+H]+=678)

### Synthesis Example 1-72

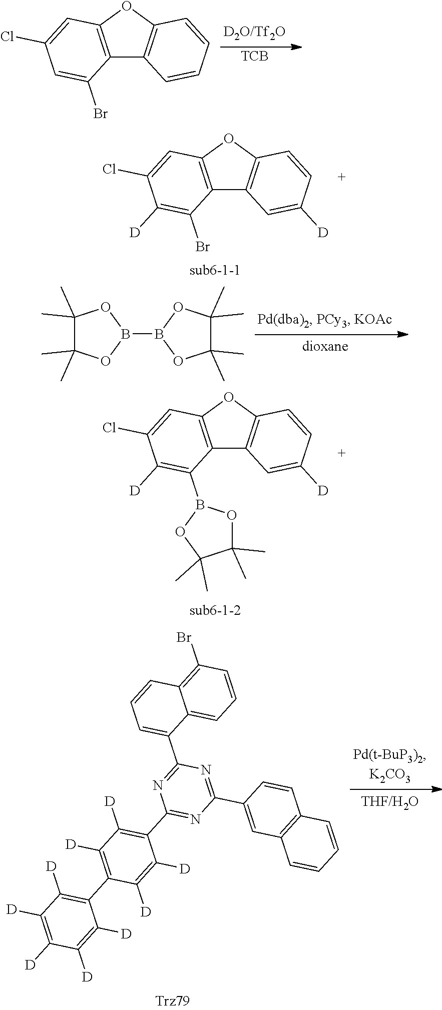

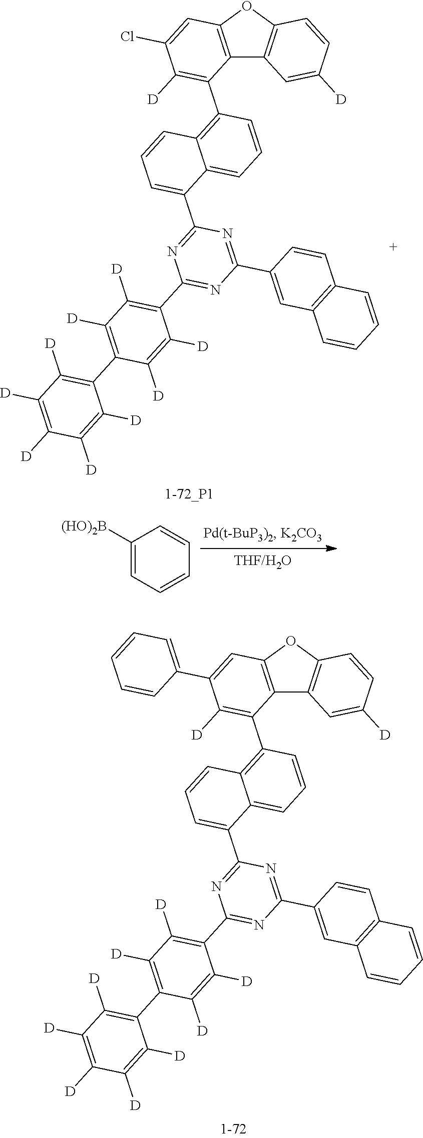 [SMILES: [2H]c1ccc2oc3cc(Cl)c([2H])c(-c4cccc5c(-c6nc(-c7ccc8ccccc8c7)nc(-c7c([2H])c([2H])c(-c8c([2H])c([2H])c([2H])c([2H])c8[2H])c([2H])c7[2H])n6)cccc45)c3c2c1 ; OB(O)c1ccccc1 ; [2H]c1ccc2oc3cc(-c4ccccc4)c([2H])c(-c4cccc5c(-c6nc(-c7ccc8ccccc8c7)nc(-c7c([2H])c([2H])c(-c8c([2H])c([2H])c([2H])c([2H])c8[2H])c([2H])c7[2H])n6)cccc45)c3c2c1]

Trifluoromethanesulfonic anhydride (30.1 g, 106.6 mmol) and deuterium oxide (10.7 g, 532.8 mmol) were added at 0° C. and stirred for 5 hours to prepare a solution. 1-Bromo-3-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-3-chlorodibenzo[b,d]furan and 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6 g of sub6-1-1. (Yield: 40%, MS: [M+H]+=283)

Sub6-1-1 (15 g, 52.9 mmol) and bis(pinacolato)diboron (14.8 g, 58.2 mmol) were added to 300 ml of 1,4-dioxane, and the mixture was stirred under reflux. Then, potassium acetate (7.8 g, 79.4 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 9.8 g of sub6-1-2. (Yield: 56%, MS: [M+H]+=331)

Sub6-1-2 (15 g, 45.4 mmol) and Trz79 (27.3 g, 47.6 mmol) were added to 300 ml of THF, and the mixture was stirred and refluxed. Then, potassium carbonate (18.8 g, 136.1 mmol) was dissolved in 56 ml of water and added thereto, and the mixture was sufficiently stirred and then bis(tri-tert-butylphosphine)palladium(0) (0.2 g, 0.5 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 21.8 g of Compound 1-72_P1. (Yield: 69%, MS: [M+H]+=698)

Compound 1-72_P1 (15 g, 21.5 mmol) and phenylboronic acid (2.8 g, 22.6 mmol) were added to 300 ml of THF, and the mixture was stirred and refluxed. Then, potassium carbonate (8.9 g, 64.5 mmol) was dissolved in 27 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11.6 g of Compound 1-72. (Yield: 73%, MS: [M+H]+=739)

### Synthesis Example 1-73

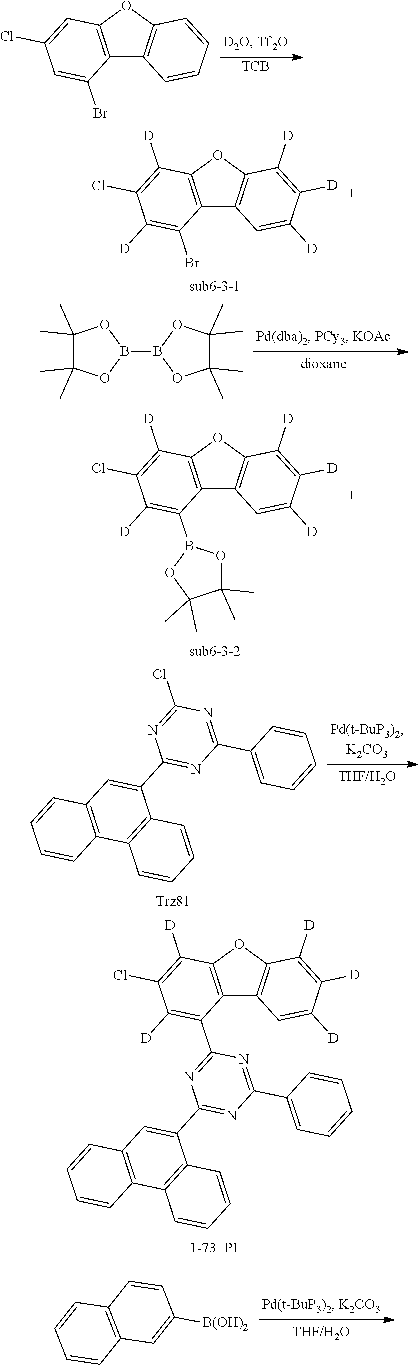

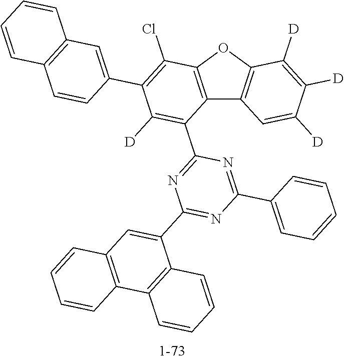 [SMILES: [2H]c1cc2c(oc3c(Cl)c(-c4ccc5ccccc5c4)c([2H])c(-c4nc(-c5ccccc5)nc(-c5cc6ccccc6c6ccccc56)n4)c32)c([2H])c1[2H]]

Trifluoromethanesulfonic anhydride (75.2 g, 266.4 mmol) and Deuterium oxide (26.7 g, 1332 mmol) were added at 0° C. and stirred for 5 hours to prepare a solution. 1-Bromo-3-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-3-chlorodibenzo[b,d]furan and 1,2,4-trichlorobenzene, and the mixture was stirred while heating to 140° C. and then keeping that temperature. After the reaction for 12 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 5.6 g of compound sub6-3-1. (Yield: 37%, MS: [M+H]+=286)

Sub6-3-1 (15 g, 52.3 mmol) and bis(pinacolato)diboron (14.6 g, 57.6 mmol) were added to 300 ml of 1,4-dioxane, and the mixture was stirred under reflux. Then, potassium acetate (7.7 g, 78.5 mmol) was added thereto, sufficiently stirred, and then bis(dibenzylideneacetone)palladium(0) (0.9 g, 1.6 mmol) and tricyclohexylphosphine (0.9 g, 3.1 mmol) were added. After the reaction for 6 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 12 g of sub6-3-2. (Yield: 69%, MS: [M+H]+=334)

Sub6-3-2 (15 g, 45 mmol) and Trz81 (17.4 g, 47.2 mmol) were added to 300 ml of THF, and the mixture was stirred and refluxed. Then, potassium carbonate (18.6 g, 134.9 mmol) was dissolved in 56 ml of water and added thereto, and the mixture was sufficiently stirred and then bis(tri-tert-butylphosphine)palladium(0) (0.2 g, 0.4 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 17.2 g of Compound 1-73_P1. (Yield: 71%, MS: [M+H]+=539)

Compound 1-73_P1 (15 g, 27.8 mmol) and naphthalen-2-ylboronic acid (5 g, 29.2 mmol) were added to 300 ml of THF, and the mixture was stirred and refluxed. Then, potassium carbonate (11.5 g, 83.5 mmol) was dissolved in 35 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.3 g of Compound 1-73. (Yield: 70%, MS: [M+H]+=631)

### Synthesis Example 1-74

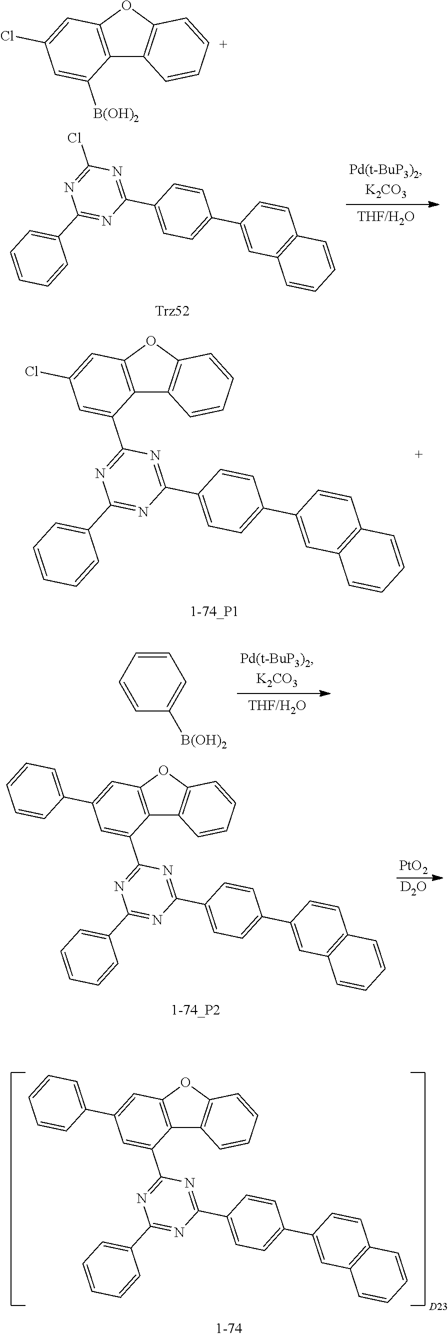

(3-Chlorodibenzo[b,d]furan-1-yl)boronic acid (15 g, 60.9 mmol) and Trz52 (25.2 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.8 g of Compound 1-74_P1. (Yield: 67%, MS: [M+H]+=560)

Compound 1-74_P1 (15 g, 26.8 mmol) and phenylboronic acid (3.4 g, 28.1 mmol) were added to 300 ml of THF, and the mixture was stirred and refluxed. Then, potassium carbonate (11.1 g, 80.3 mmol) was dissolved in 33 ml of water and added thereto, and the mixture was sufficiently stirred and then bis(tri-tert-butylphosphine)palladium(0) (0.1 g, 0.3 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 10.9 g of Compound 1-74_P2. (Yield: 68%, MS: [M+H]+=602)

Compound 1-74_P2 (10 g, 16.6 mmol), PtO₂ (1.1 g, 5 mmol), and D₂O (83 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO4 and concentrated, and then the sample was purified by silica gel column chromatography to prepare 4 g of Compound 1-74. (Yield: 39%, MS: [M+H]+=626)

### Synthesis Example 1-75

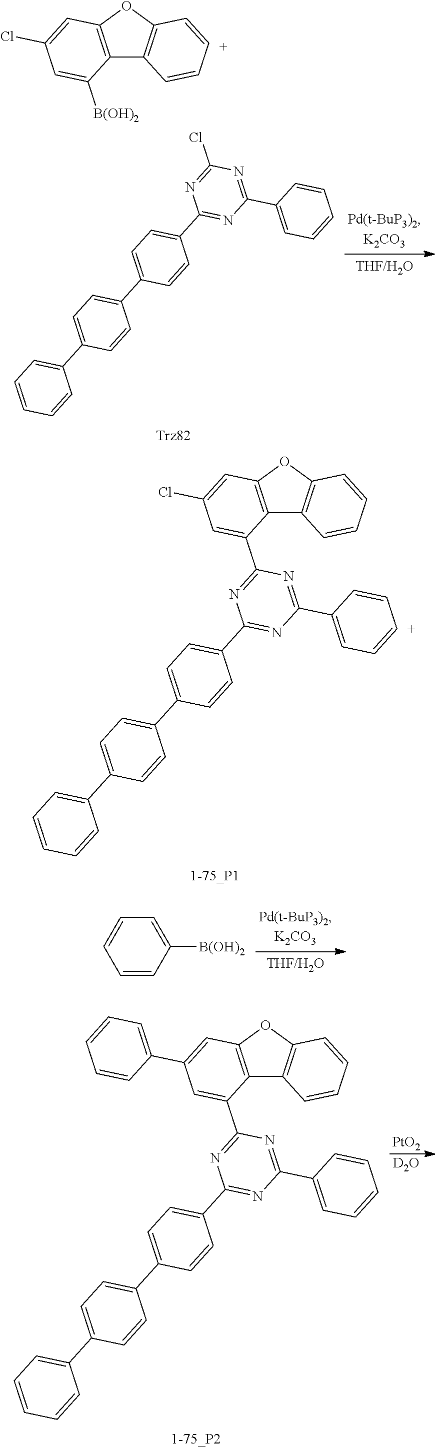

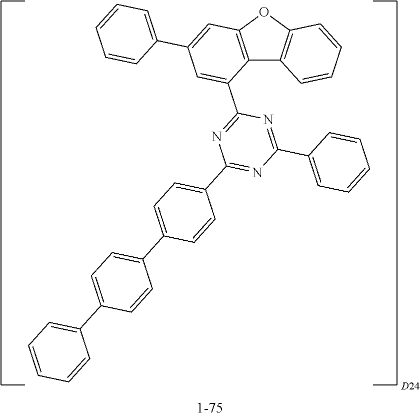

(2-Chlorodibenzo[b,d]furan-1-yl)boronic acid (15 g, 60.9 mmol) and Trz82 (26.8 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.3 g of Compound 1-75_P1. (Yield: 71%, MS: [M+H]+=586)

Compound 1-75_P1 (15 g, 25.6 mmol) and phenylboronic acid (3.3 g, 26.9 mmol) were added to 300 ml of THF, and the mixture was stirred and refluxed. Then, potassium carbonate (10.6 g, 76.8 mmol) was dissolved in 32 ml of water and added thereto, and the mixture was sufficiently stirred and then bis(tri-tert-butylphosphine)palladium(0) (0.1 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12 g of Compound 1-75_P2. (Yield: 75%, MS: [M+H]+=628)

Compound 1-75_P2 (10 g, 15.9 mmol), PtO₂ (1.1 g, 4.8 mmol) and D₂O (80 ml) were added to a shaker tube, and then the tube was sealed and heated at 250° C. and 600 psi for 12 hours. After completion of the reaction, chloroform was added, and the reaction solution was transferred to a separatory funnel, and extracted. The extract was dried over MgSO4 and concentrated, and then the sample was purified by silica gel column chromatography to prepare 4 g of Compound 1-75. (Yield: 39%, MS: [M+H]+=653)

### Synthesis Example 1-76

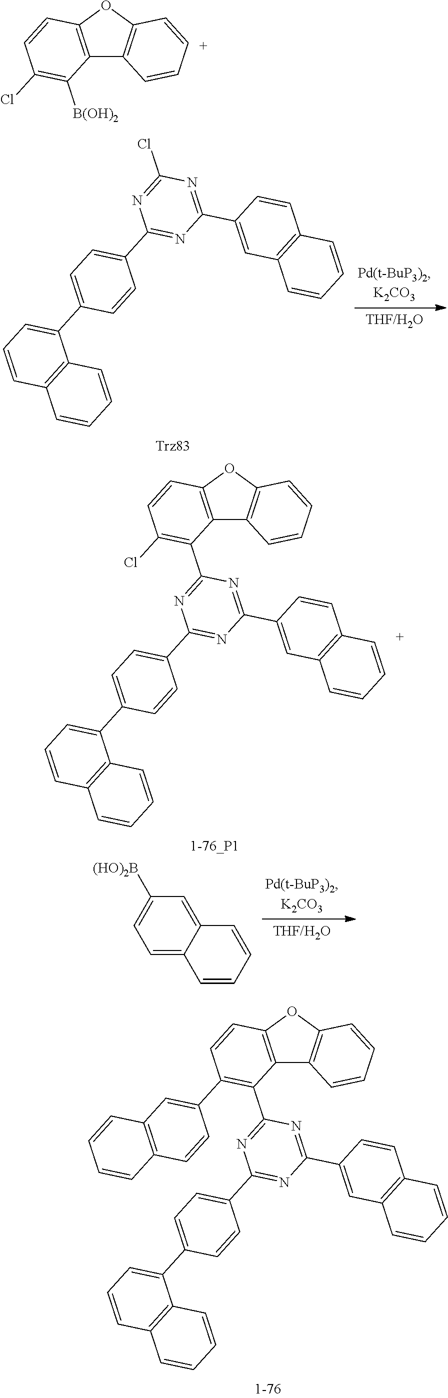

(3-Chlorodibenzo[b,d]furan-1-yl)boronic acid (15 g, 60.9 mmol) and Trz83 (28.4 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.8 g of Compound 1-76_P1. (Yield: 67%, MS: [M+H]+=610)

Compound 1-76_P1 (15 g, 24.6 mmol) and naphthalen-2-ylboronic acid (4.4 g, 25.8 mmol) were added to 300 ml of THF, and the mixture was stirred and refluxed. Then, potassium carbonate (10.2 g, 73.8 mmol) was dissolved in 31 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.1 g of Compound 1-76. (Yield: 70%, MS: [M+H]+=702)

### Synthesis Example 1-77

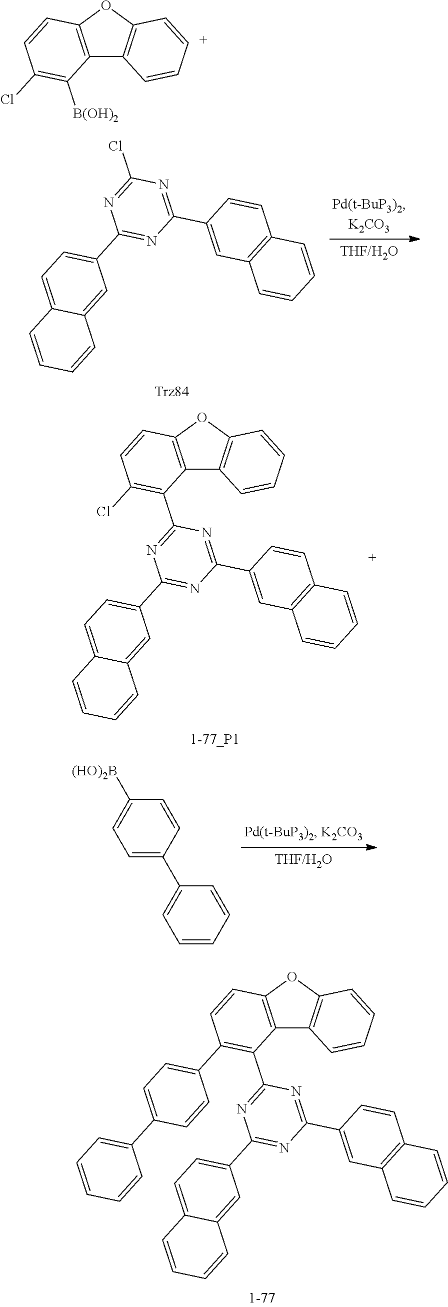 [SMILES: OB(O)c1c(Cl)ccc2oc3c(c12)C=C[C+]=C3 ; Clc1nc(-c2ccc3ccccc3c2)nc(-c2ccc3ccccc3c2)n1 ; Clc1ccc2oc3ccccc3c2c1-c1nc(-c2ccc3ccccc3c2)nc(-c2ccc3ccccc3c2)n1 ; OB(O)c1ccc(-c2ccccc2)cc1 ; c1ccc(-c2ccc(-c3ccc4oc5ccccc5c4c3-c3nc(-c4ccc5ccccc5c4)nc(-c4ccc5ccccc5c4)n3)cc2)cc1]

(2-Chlorodibenzo[b,d]furan-1-yl)boronic acid (15 g, 60.9 mmol) and Trz84 (23.5 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.4 g of Compound 1-77_P1. (Yield: 72%, MS: [M+H]+=534)

Compound 1-77_P1 (15 g, 28.1 mmol) and [1,1′-biphenyl]-4-ylboronic acid (5.8 g, 29.5 mmol) were added to 300 ml of THF, and the mixture was stirred and refluxed. Then, potassium carbonate (11.6 g, 84.3 mmol) was dissolved in 35 ml of water and added thereto, and the mixture was sufficiently stirred and then bis(tri-tert-butylphosphine)palladium(0) (0.1 g, 0.3 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.1 g of Compound 1-77. (Yield: 66%, MS: [M+H]+=652)

### Synthesis Example 1-78

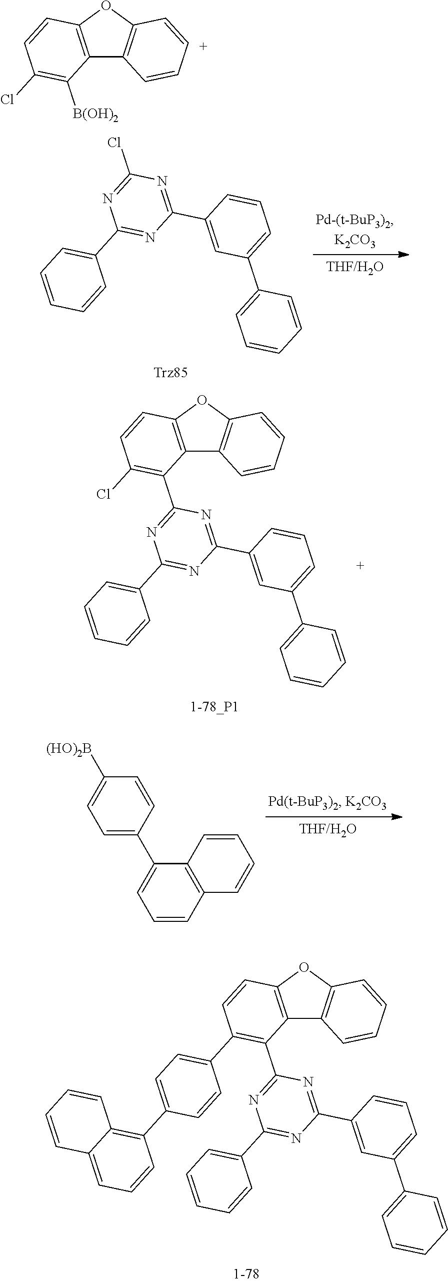 [SMILES: OB(O)c1c(Cl)ccc2oc3c(c12)C=C[C+]=C3 ; Clc1nc(-c2ccccc2)nc(-c2cccc(-c3ccccc3)c2)n1 ; Clc1ccc2oc3ccccc3c2c1-c1nc(-c2ccccc2)nc(-c2cccc(-c3ccccc3)c2)n1 ; OB(O)c1ccc(-c2cccc3ccccc23)cc1 ; c1ccc(-c2cccc(-c3nc(-c4ccccc4)nc(-c4c(-c5ccc(-c6cccc7ccccc67)cc5)ccc5oc6ccccc6c45)n3)c2)cc1]

(2-Chlorodibenzo[b,d]furan-1-yl)boronic acid (15 g, 60.9 mmol) and Trz85 (22 g, 63.9 mmol) were added to 300 ml of THF, and the mixture was stirred and refluxed. Then, potassium carbonate (25.2 g, 182.6 mmol) was dissolved in 76 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 20.1 g of Compound 1-78_P1. (Yield: 65%, MS: [M+H]+=510)

Compound 1-78_P1 (15 g, 29.4 mmol) and (4-(naphthalen-1-yl)phenyl)boronic acid (7.7 g, 30.9 mmol) were added to 300 ml of THF, and the mixture was stirred and refluxed. Then, potassium carbonate (12.2 g, 88.2 mmol) was dissolved in 37 ml of water and added thereto, and the mixture was sufficiently stirred and then bis(tri-tert-butylphosphine)palladium(0) (0.2 g, 0.3 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.9 g of Compound 1-78. (Yield: 65%, MS: [M+H]+=678)

### Synthesis Example 1-79

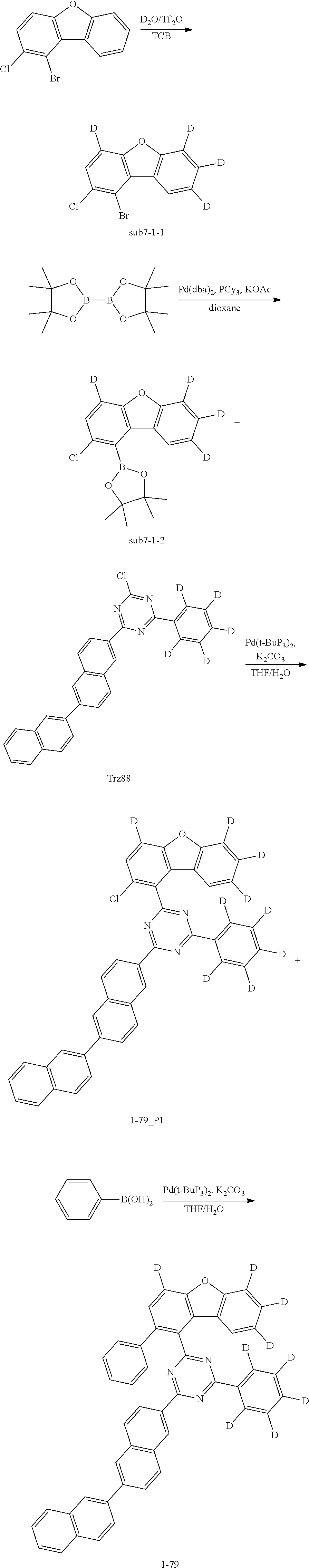

Trifluoromethanesulfonic anhydride (60.1 g, 213.1 mmol) and deuterium oxide (21.4 g, 1065.6 mmol) were added at 0° C. and stirred for 5 hours to prepare a solution. 1-Bromo-2-chlorodibenzo[b,d]furan (15 g, 53.3 mmol) was added to 120 ml of 1,2,4-trichlorobenzene, and the mixture was stirred. Then, the prepared mixed solution of trifluoromethanesulfonic anhydride and deuterium oxide was slowly added dropwise to the mixed solution of 1-bromo-2-chlorodibenzo[b,d]furan and 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by silica gel column chromatography to prepare 6.1 g of sub7-1-1. (Yield: 40%, MS: [M+H]+=285)

Sub7-1-1 (15 g, 52.5 mmol) and bis(pinacolato)diboron (14.7 g, 57.8 mmol) were added to 300 ml of 1,4-dioxane, and the mixture was stirred under reflux. Then, potassium acetate (7.7 g, 78.8 mmol) was added thereto, sufficiently stirred, and then bis(dibenzylideneacetone)palladium(0) (0.9 g, 1.6 mmol) and tricyclohexylphosphine (0.9 g, 3.2 mmol) were added. After the reaction for 5 hours, the reaction mixture was cooled to room temperature, the organic layer was separated using chloroform and water, and then the organic layer was distilled. This was dissolved again in chloroform, washed twice with water, and the organic layer was then separated. Anhydrous magnesium sulfate was added thereto, stirred, then filtered, and the filtrate was distilled under reduced pressure. The concentrated compound was purified by silica gel column chromatography to prepare 10.5 g of sub7-1-2. (Yield: 60%, MS: [M+H]+=333)

Sub7-1-2 (15 g, 45.1 mmol) and Trz88 (21.3 g, 47.4 mmol) were added to 300 ml of THF, and the mixture was stirred and refluxed. Then, potassium carbonate (18.7 g, 135.3 mmol) was dissolved in 56 ml of water and added thereto, and the mixture was sufficiently stirred and then bis(tri-tert-butylphosphine)palladium(0) (0.2 g, 0.5 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 18.1 g of Compound 1-79_P1. (Yield: 65%, MS: [M+H]+=619)

Compound 1-79_P1 (15 g, 24.2 mmol) and phenylboronic acid (3.1 g, 25.4 mmol) were added to 300 ml of THF, and the mixture was stirred and refluxed. Then, potassium carbonate (10 g, 72.7 mmol) was dissolved in 30 ml of water and added thereto, and the mixture was sufficiently stirred and then bis(tri-tert-butylphosphine)palladium(0) (0.1 g, 0.2 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 11 g of Compound 1-79. (Yield: 69%, MS: [M+H]+=661)

### Preparation of Compound Represented by Chemical Formula 2

### Preparation Example 1

### Preparation of Compound AA

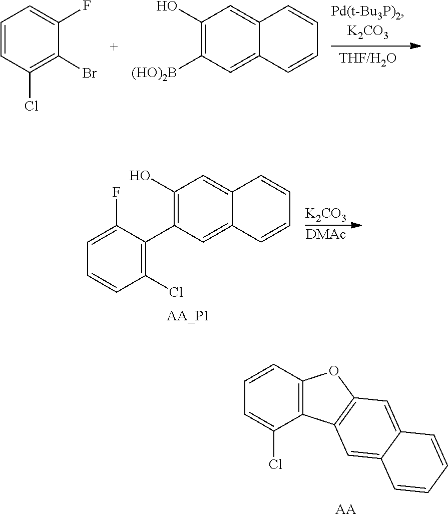

2-Bromo-1-chloro-3-fluorobenzene (15 g, 71.6 mmol) and (3-hydroxynaphthalen-2-yl) boronic acid (14.1 g, 75.2 mmol) were added to 300 ml of THF, and the mixture was stirred and refluxed. Then, potassium carbonate (29.7 g, 214.9 mmol) was dissolved in 89 ml of water and added thereto, and the mixture was sufficiently stirred and then bis(tri-tert-butylphosphine)palladium(0) (0.4 g, 0.7 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 12.7 g of Compound AA_P1. (Yield: 65%, MS: [M+H]+=273)

Compound AA_P1 (15 g, 55 mmol) and potassium carbonate (22.8 g, 165 mmol) were added to 150 ml of DMAc, and the mixture was stirred and refluxed. After the reaction for 2 hours, the reaction mixture was cooled to room temperature, poured into 300 ml of water and solidified. This was filtered and then dissolved in chloroform, washed twice with water, the organic layer was separated, anhydrous magnesium sulfate was added, stirred, filtered, and the filtrate was distilled under reduced pressure. The concentrated compound was purified by silica gel column chromatography to prepare 8.6 g of Compound AA. (Yield 62%, MS: [M+H]+=253)

### Preparation Example 2

### Preparation of Compound AB

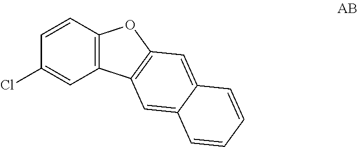 [SMILES: Clc1ccc2oc3cc4ccccc4cc3c2c1]

Compound AB was prepared in the same manner as in Preparation Example 1, except that 2-bromo-4-chloro-1-fluorobenzene was used instead of 2-bromo-1-chloro-3-fluorobenzene.

### Preparation Example 3

### Preparation of Compound AC

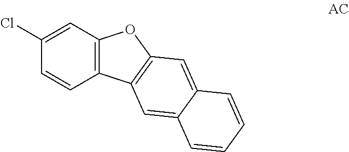 [SMILES: Clc1ccc2c(c1)oc1cc3ccccc3cc12]

Compound AC was prepared in the same manner as in Preparation Example 1, except that 1-bromo-4-chloro-2-fluorobenzene was used instead of 2-bromo-1-chloro-3-fluorobenzene.

### Preparation Example 4

### Preparation of Compound AD

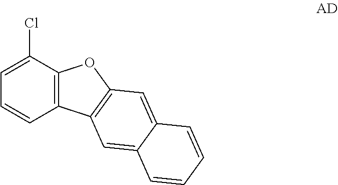 [SMILES: Clc1cccc2c1oc1cc3ccccc3cc12]

Compound AD was prepared in the same manner as in Preparation Example 1, except that 1-bromo-3-chloro-2-fluorobenzene was used instead of 2-bromo-1-chloro-3-fluorobenzene.

### Preparation Example 5

### Preparation of Compound AE

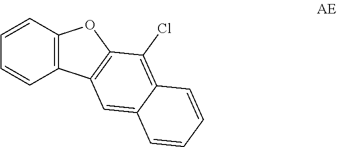 [SMILES: Clc1c2ccccc2cc2c1oc1ccccc12]

Compound AE was prepared in the same manner as in Preparation Example 1, except that 1-bromo-2-fluorobenzene was used instead of 2-bromo-1-chloro-3-fluorobenzene, and (4-chloro-3-hydroxynaphthalen-2-yl) boronic acid was used instead of (3-hydroxynaphthalen-2-yl)boronic acid.

### Preparation Example 6

### Preparation of Compound AF

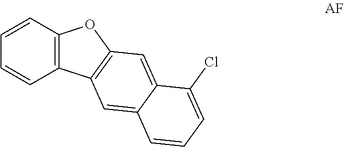 [SMILES: Clc1cccc2cc3c(cc12)oc1ccccc13]

Compound AF was prepared in the same manner as in Preparation Example 1, except that 1-bromo-2-fluorobenzene was used instead of 2-bromo-1-chloro-3-fluorobenzene, and (5-chloro-3-hydroxynaphthalen-2-yl)boronic acid was used instead of (3-hydroxynaphthalen-2-yl)boronic acid.

### Preparation Example 7

### Preparation of Compound AG

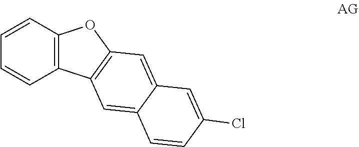 [SMILES: Clc1ccc2cc3c(cc2c1)oc1ccccc13]

Compound AG was prepared in the same manner as in Preparation Example 1, except that 1-bromo-2-fluorobenzene was used instead of 2-bromo-1-chloro-3-fluorobenzene, and (6-chloro-3-hydroxynaphthalen-2-yl)boronic acid was used instead of (3-hydroxynaphthalen-2-yl)boronic acid.

### Preparation Example 8

### Preparation of Compound AH

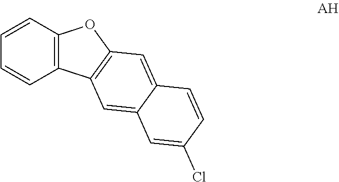 [SMILES: Clc1ccc2cc3oc4ccccc4c3cc2c1]

Compound AH was prepared in the same manner as in Preparation 10 Example 1, except that 1-bromo-2-fluorobenzene was used instead of 2-bromo-1-chloro-3-fluorobenzene, and (7-chloro-3-hydroxynaphthalen-2-yl)boronic acid was used instead of (3-hydroxynaphthalen-2-yl)boronic acid.

### Preparation Example 9

### Preparation of Compound AI

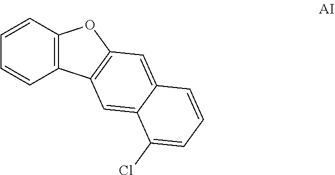 [SMILES: Clc1cccc2cc3oc4ccccc4c3cc12]

Compound AI was prepared in the same manner as in Preparation Example 1, except that 1-bromo-2-fluorobenzene was used instead of 2-bromo-1-chloro-3-fluorobenzene, and (8-chloro-3-hydroxynaphthalen-2-yl)boronic acid was used instead of (3-hydroxynaphthalen-2-yl)boronic acid.

### Preparation Example 10

### Preparation of Compound AJ

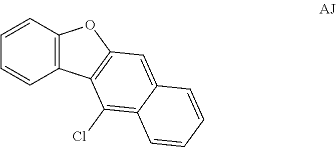 [SMILES: Clc1c2ccccc2cc2oc3ccccc3c12]

Compound AJ was prepared in the same manner as in Preparation Example 1, except that 1-bromo-2-fluorobenzene was used instead of 2-bromo-1-chloro-3-fluorobenzene, and (1-chloro-3-hydroxynaphthalen-2-yl)boronic acid was used instead of (3-hydroxynaphthalen-2-yl)boronic acid.

### Preparation Example 11

### Preparation of Compound BA

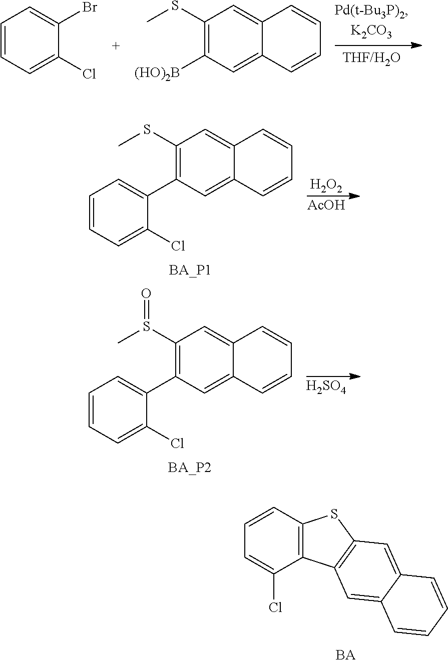

1-Bromo-2-chlorobenzene (15 g, 78.3 mmol) and (3-(methylthio)naphthalen-2-yl)boronic acid (17.9 g, 82.3 mmol) were added to 300 ml of THF, and the mixture was stirred and refluxed. Then, potassium carbonate (32.5 g, 235 mmol) was dissolved in 97 ml of water and added thereto, and the mixture was sufficiently stirred and then bis(tri-tert-butylphosphine)palladium(0) (0.4 g, 0.8 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 15.8 g of Compound BA_P1. (Yield: 71%, MS: [M+H]+=286) Compound BA_P1 (15 g, 55 mmol) and hydrogen peroxide (2.8 g, 82.5 mmol) were added to 150 ml of acetic acid, and the mixture was stirred and refluxed. After the reaction for 3 hours, the reaction mixture was poured into 300 ml of water, and crystals were precipitated and filtered. The filtered solid was dissolved in chloroform, washed twice with water, the organic layer was separated, anhydrous magnesium sulfate was added, stirred, then filtered, and the filtrate was distilled under reduced pressure. The concentrated compound was purified by silica gel column chromatography to prepare 8.7 g of Compound BA_P2. (Yield 53%, MS: [M+H]+=301)

Compound BA_P2 (15 g, 49.9 mmol) was added to 150 ml of H₂SO₄, and the mixture was stirred and refluxed. When the reaction was completed for 2 hours, the reaction mixture was poured into 300 ml of water, and crystals were precipitated and filtered. The filtered solid was dissolved in chloroform, washed twice with water, the organic layer was separated, anhydrous magnesium sulfate was added, stirred, then filtered, and the filtrate was distilled under reduced pressure. The concentrated compound was purified by silica gel column chromatography to prepare 7.8 g of Compound BA. (Yield 58%, MS: [M+H]+=269)

### Preparation Example 12

### Preparation of Compound BB

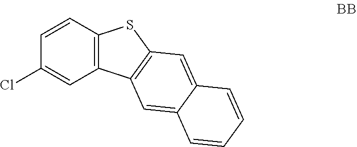 [SMILES: Clc1ccc2sc3cc4ccccc4cc3c2c1]

Compound BB was prepared in the same manner as in Preparation Example 11, except that 1-bromo-3-chlorobenzene was used instead of 1-bromo-2-chlorobenzene.

### Preparation Example 13

### Preparation of Compound BC

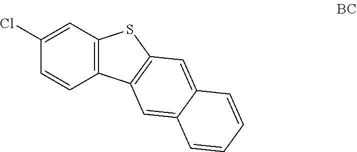 [SMILES: Clc1ccc2c(c1)sc1cc3ccccc3cc12]

Compound BC was prepared in the same manner as in Preparation Example 11, except that 1-bromo-4-chlorobenzene was used instead of 1-bromo-2-chlorobenzene.

### Preparation Example 14

### Preparation of Compound BD

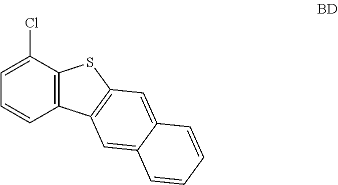 [SMILES: Clc1cccc2c1sc1cc3ccccc3cc12]

Compound BD was prepared in the same manner as in Preparation Example 11, except that 1-bromo-3-chlorobenzene was used instead of 1-bromo-2-chlorobenzene.

### Preparation Example 15

### Preparation of Compound BE

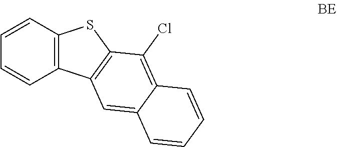 [SMILES: Clc1c2ccccc2cc2c1sc1ccccc12]

Compound BE was prepared in the same manner as in Preparation Example 11, except that bromobenzene was used instead of 1-bromo-2-chlorobenzene, and (4-chloro-3-(methylthio)naphthalen-2-yl)boronic acid was used instead of (3-(methylthio)naphthalen-2-yl)boronic acid.

### Preparation Example 16

### Preparation of Compound BF

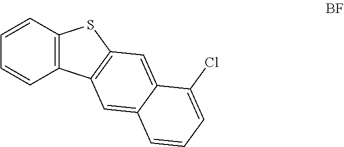 [SMILES: Clc1cccc2cc3c(cc12)sc1ccccc13]

Compound BF was prepared in the same manner as in Preparation Example 11, except that bromobenzene was used instead of 1-bromo-2-chlorobenzene, and (5-chloro-3-(methylthio)naphthalen-2-yl)boronic acid was used instead of (3-(methylthio)naphthalen-2-yl)boronic acid.

### Preparation Example 17

### Preparation of Compound BG

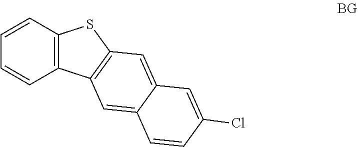 [SMILES: Clc1ccc2cc3c(cc2c1)sc1ccccc13]

Compound BG was prepared in the same manner as in Preparation Example 11, except that bromobenzene was used instead of 1-bromo-2-chlorobenzene, and (6-chloro-3-(methylthio)naphthalen-2-yl)boronic acid was used instead of (3-(methylthio)naphthalen-2-yl)boronic acid.

### Preparation Example 18

### Preparation of Compound BH

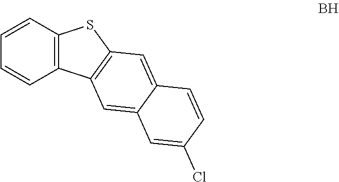 [SMILES: Clc1ccc2cc3sc4ccccc4c3cc2c1]

Compound BH was prepared in the same manner as in Preparation Example 11, except that bromobenzene was used instead of 1-bromo-2-chlorobenzene, and (7-chloro-3-(methylthio)naphthalen-2-yl)boronic acid was used instead of (3-(methylthio)naphthalen-2-yl)boronic acid.

### Preparation Example 19

### Preparation of Compound BI

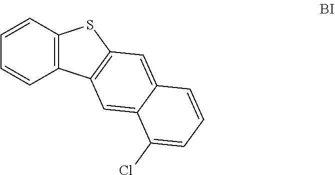 [SMILES: Clc1cccc2cc3sc4ccccc4c3cc12]

Compound BI was prepared in the same manner as in Preparation Example 11, except that bromobenzene was used instead of 1-bromo-2-chlorobenzene, and (8-chloro-3-(methylthio)naphthalen-2-yl)boronic acid was used instead of (3-(methylthio)naphthalen-2-yl)boronic acid.

### Preparation Example 20

### Preparation of Compound BJ

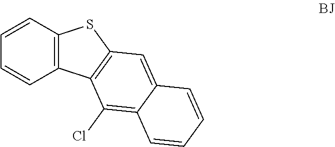 [SMILES: Clc1c2ccccc2cc2sc3ccccc3c12]

Compound BJ was prepared in the same manner as in Preparation Example 11, except that bromobenzene was used instead of 1-bromo-2-chlorobenzene, and (1-chloro-3-(methylthio)naphthalen-2-yl)boronic acid was used instead of (3-(methylthio)naphthalen-2-yl)boronic acid.

### Synthesis Example 2-1

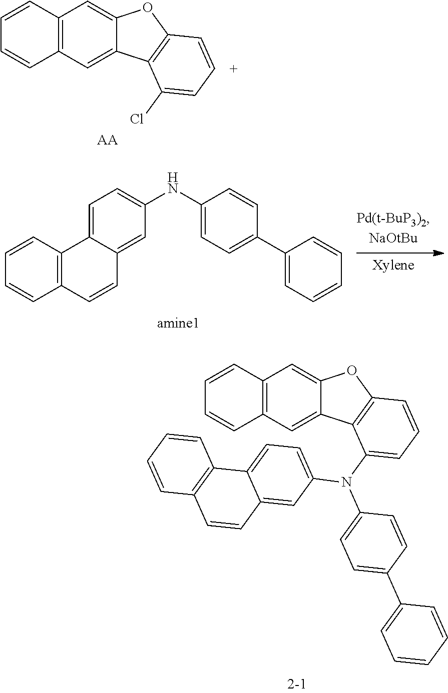 [SMILES: Clc1cccc2oc3cc4ccccc4cc3c12 ; "AA" ; c1ccc(-c2ccc(Nc3ccc4c(ccc5ccccc54)c3)cc2)cc1 ; c1ccc(-c2ccc(N(c3ccc4c(ccc5ccccc54)c3)c3cccc4oc5cc6ccccc6cc5c34)cc2)cc1]

Compound AA (10 g, 39.6 mmol), amine 1 (13.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.8 g of Compound 2-1. (Yield: 62%, MS: [M+H]+=562)

### Synthesis Example 2-2

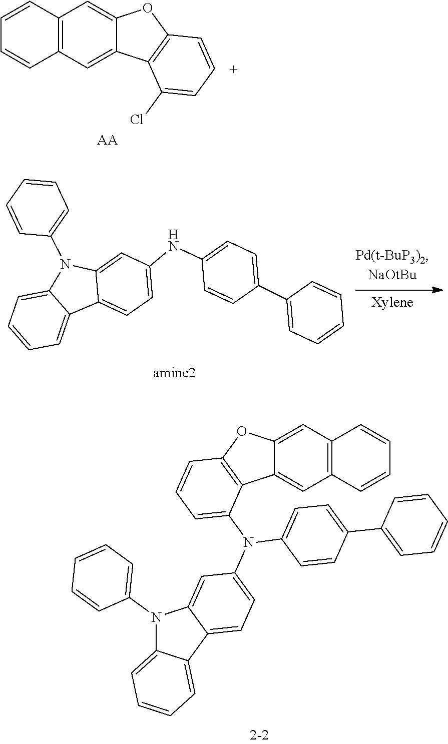 [SMILES: Clc1cccc2oc3cc4ccccc4cc3c12 ; "AA" ; c1ccc(-c2ccc(Nc3ccc4c5ccccc5n(-c5ccccc5)c4c3)cc2)cc1 ; c1ccc(-c2ccc(N(c3ccc4c5ccccc5n(-c5ccccc5)c4c3)c3cccc4oc5cc6ccccc6cc5c34)cc2)cc1]

Compound AA (10 g, 39.6 mmol), amine 2 (16.2 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.6 g of Compound 2-2. (Yield: 67%, MS: [M+H]+=627)

### Synthesis Example 2-3

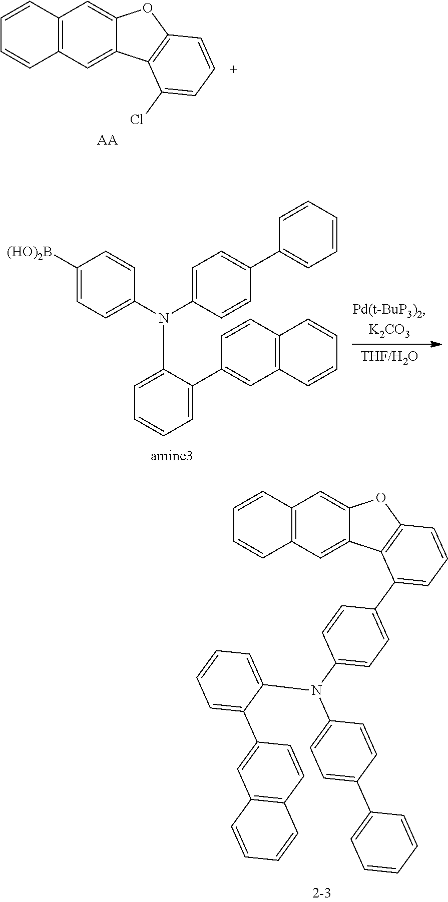 [SMILES: Clc1cccc2oc3cc4ccccc4cc3c12 ; "AA" ; OB(O)c1ccc(N(c2ccc(-c3ccccc3)cc2)c2ccccc2-c2ccc3ccccc3c2)cc1 ; c1ccc(-c2ccc(N(c3ccc(-c4cccc5oc6cc7ccccc7cc6c45)cc3)c3ccccc3-c3ccc4ccccc4c3)cc2)cc1]

Compound AA (15 g, 59.4 mmol) and amine 3 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.1 g of Compound 2-3. (Yield: 74%, MS: [M+H]+=664)

### Synthesis Example 2-4

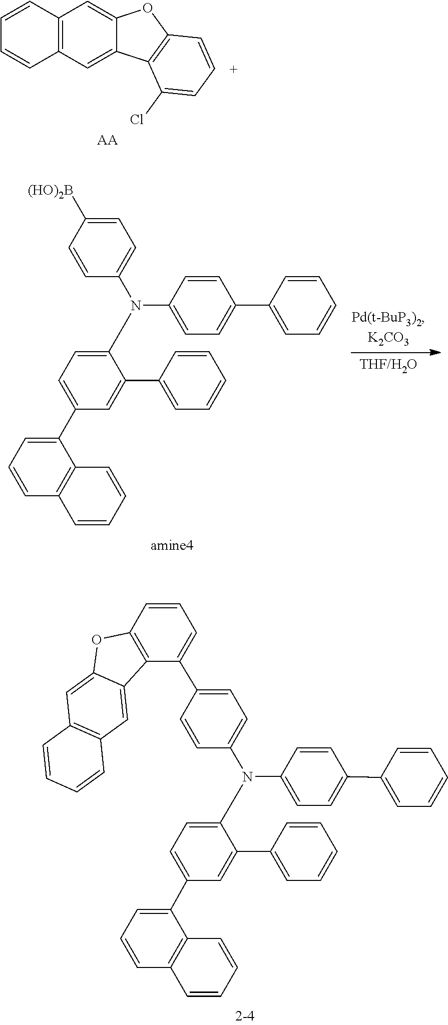 [SMILES: Clc1cccc2oc3cc4ccccc4cc3c12 ; "AA" ; OB(O)c1ccc(N(c2ccc(-c3ccccc3)cc2)c2ccc(-c3cccc4ccccc34)cc2-c2ccccc2)cc1 ; c1ccc(-c2ccc(N(c3ccc(-c4cccc5oc6cc7ccccc7cc6c45)cc3)c3ccc(-c4cccc5ccccc45)cc3-c3ccccc3)cc2)cc1]

Compound AA (15 g, 59.4 mmol) and amine 4 (35.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.5 g of Compound 2-4 (Yield: 65%, MS: [M+H]+=740)

### Synthesis Example 2-5

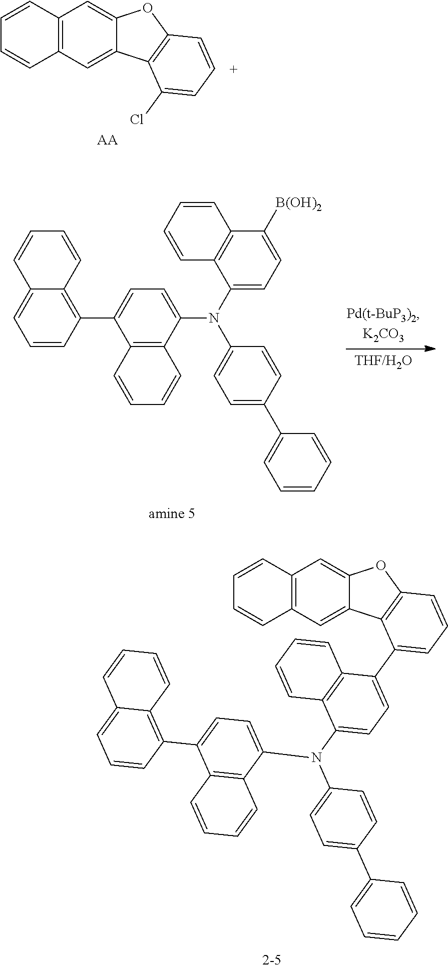 [SMILES: Clc1cccc2oc3cc4ccccc4cc3c12 ; "AA" ; OB(O)c1ccc(N(c2ccc(-c3ccccc3)cc2)c2ccc(-c3cccc4ccccc34)c3ccccc23)c2ccccc12 ; c1ccc(-c2ccc(N(c3ccc(-c4cccc5ccccc45)c4ccccc34)c3ccc(-c4cccc5oc6cc7ccccc7cc6c45)c4ccccc34)cc2)cc1]

Compound AA (15 g, 59.4 mmol) and amine 5 (36.9 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 33.5 g of Compound 2-5. (Yield: 74%, MS: [M+H]+=764)

### Synthesis Example 2-6

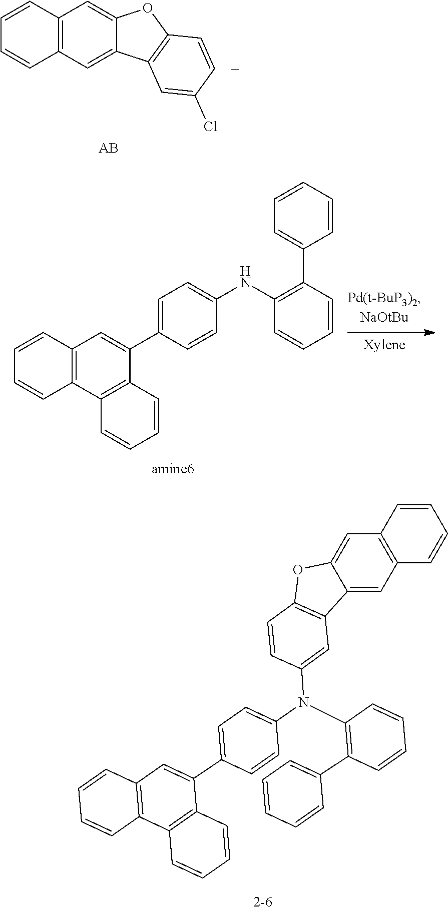 [SMILES: Clc1ccc2oc3cc4ccccc4cc3c2c1 ; "AB" ; c1ccc(-c2ccccc2Nc2ccc(-c3cc4ccccc4c4ccccc34)cc2)cc1 ; c1ccc(-c2ccccc2N(c2ccc(-c3cc4ccccc4c4ccccc34)cc2)c2ccc3oc4cc5ccccc5cc4c3c2)cc1]

Compound AB (10 g, 39.6 mmol), amine 6 (16.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.4 g of Compound 2-6. (Yield: 65%, MS: [M+H]+=638)

### Synthesis Example 2-7

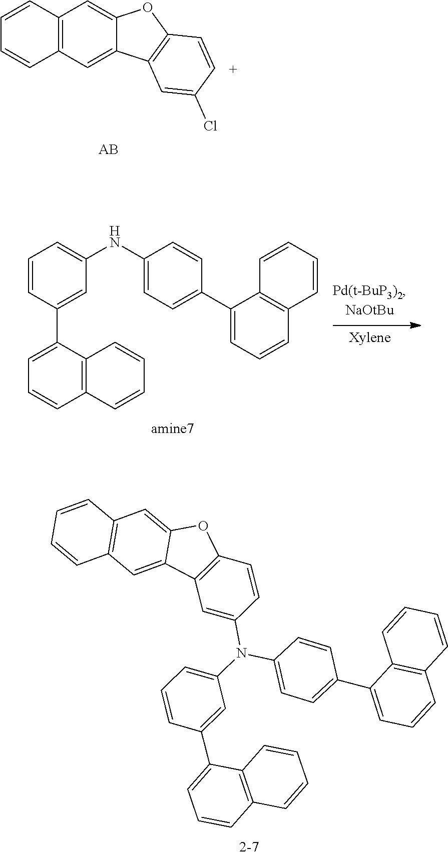 [SMILES: Clc1ccc2oc3cc4ccccc4cc3c2c1 ; c1cc(Nc2ccc(-c3cccc4ccccc34)cc2)cc(-c2cccc3ccccc23)c1 ; c1cc(-c2cccc3ccccc23)cc(N(c2ccc(-c3cccc4ccccc34)cc2)c2ccc3oc4cc5ccccc5cc4c3c2)c1]

Compound AB (10 g, 39.6 mmol), amine 7 (16.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.1 g of Compound 2-7. (Yield: 68%, MS: [M+H]+=638)

### Synthesis Example 2-8

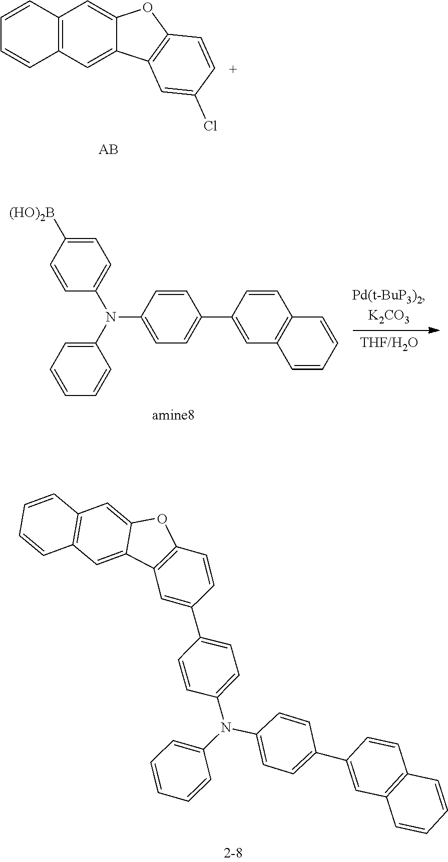 [SMILES: Clc1ccc2oc3cc4ccccc4cc3c2c1 ; OB(O)c1ccc(N(c2ccccc2)c2ccc(-c3ccc4ccccc4c3)cc2)cc1 ; c1ccc(N(c2ccc(-c3ccc4ccccc4c3)cc2)c2ccc(-c3ccc4oc5cc6ccccc6cc5c4c3)cc2)cc1]

Compound AB (15 g, 59.4 mmol) and amine 8 (25.9 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.1 g of Compound 2-8. (Yield: 69%, MS: [M+H]+=588)

### Synthesis Example 2-9

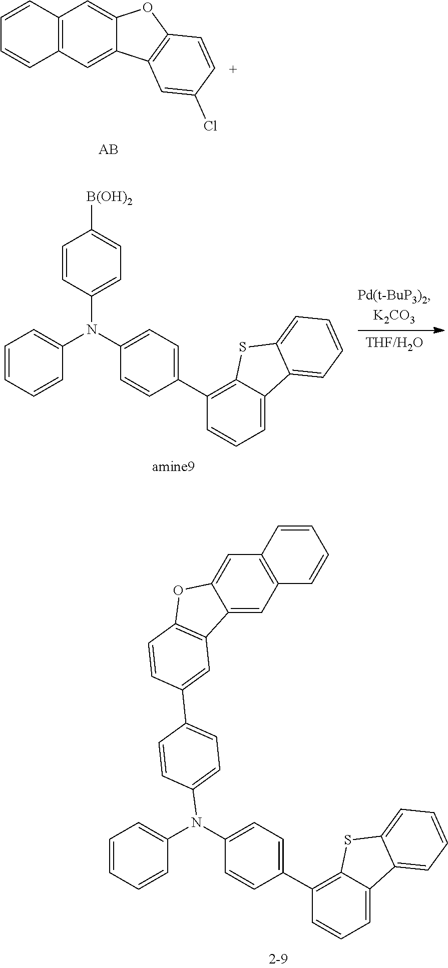 [SMILES: Clc1ccc2oc3cc4ccccc4cc3c2c1 ; OB(O)c1ccc(N(c2ccccc2)c2ccc(-c3cccc4c3sc3ccccc34)cc2)cc1 ; c1ccc(N(c2ccc(-c3ccc4oc5cc6ccccc6cc5c4c3)cc2)c2ccc(-c3cccc4c3sc3ccccc34)cc2)cc1]

Compound AB (15 g, 59.4 mmol) and amine 9 (29.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.9 g of Compound 2-9. (Yield: 73%, MS: [M+H]+=644)

### Synthesis Example 2-10

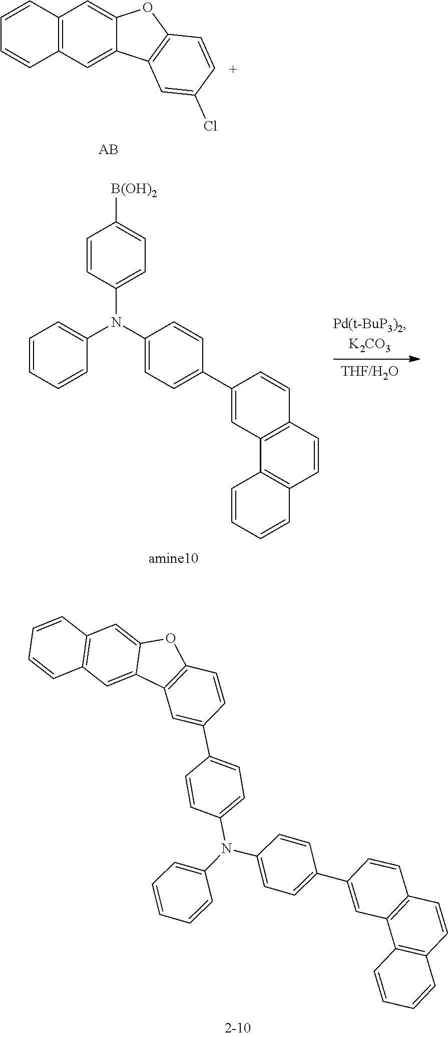 [SMILES: Clc1ccc2oc3cc4ccccc4cc3c2c1 ; OB(O)c1ccc(N(c2ccccc2)c2ccc(-c3ccc4ccc5ccccc5c4c3)cc2)cc1 ; c1ccc(N(c2ccc(-c3ccc4ccc5ccccc5c4c3)cc2)c2ccc(-c3ccc4oc5cc6ccccc6cc5c4c3)cc2)cc1]

Compound AB (15 g, 59.4 mmol) and amine 10 (29 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25 g of Compound 2-10. (Yield: 66%, MS: [M+H]+=638)

### Synthesis Example 2-11

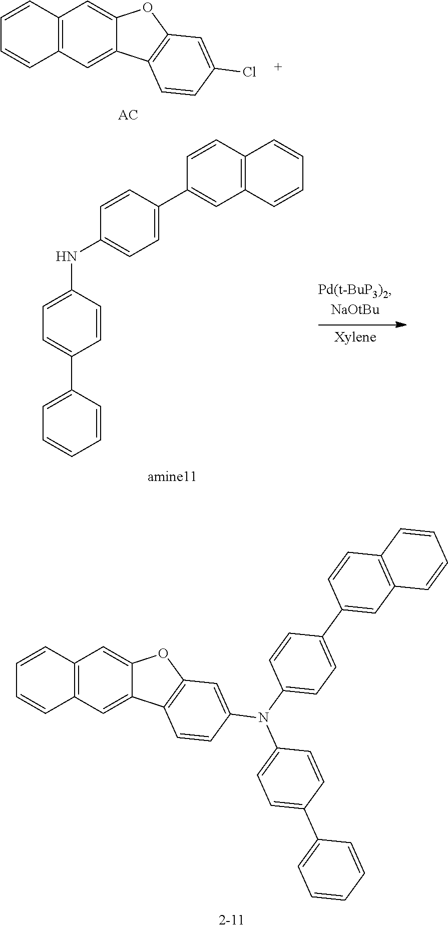 [SMILES: Clc1ccc2c(c1)oc1cc3ccccc3cc12 ; c1ccc(-c2ccc(Nc3ccc(-c4ccc5ccccc5c4)cc3)cc2)cc1 ; c1ccc(-c2ccc(N(c3ccc(-c4ccc5ccccc5c4)cc3)c3ccc4c(c3)oc3cc5ccccc5cc34)cc2)cc1]

Compound AC (10 g, 39.6 mmol), amine 11 (14.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.6 g of Compound 2-11. (Yield: 67%, MS: [M+H]+=588)

### Synthesis Example 2-12

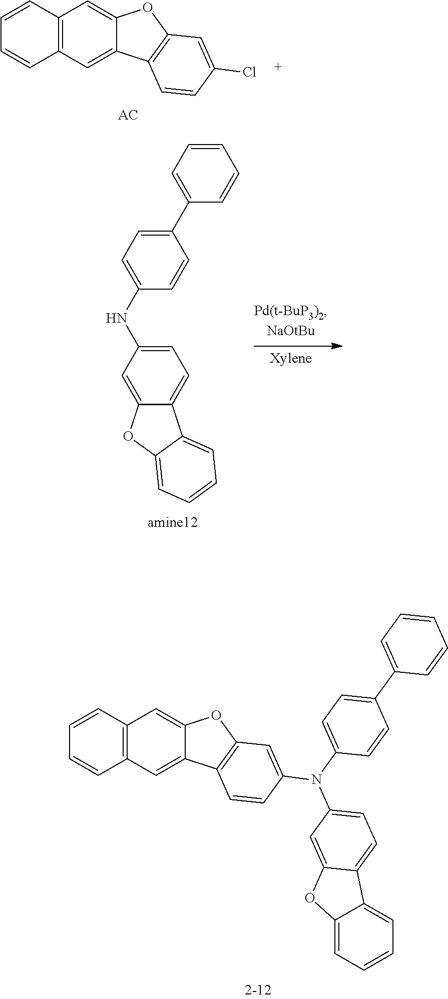 [SMILES: Clc1ccc2c(c1)oc1cc3ccccc3cc12 ; c1ccc(-c2ccc(Nc3ccc4c(c3)oc3ccccc34)cc2)cc1 ; c1ccc(-c2ccc(N(c3ccc4c(c3)oc3ccccc34)c3ccc4c(c3)oc3cc5ccccc5cc34)cc2)cc1]

Compound AC (10 g, 39.6 mmol), amine 12 (13.3 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound 2-12. (Yield: 67%, MS: [M+H]+=552)

### Synthesis Example 2-13

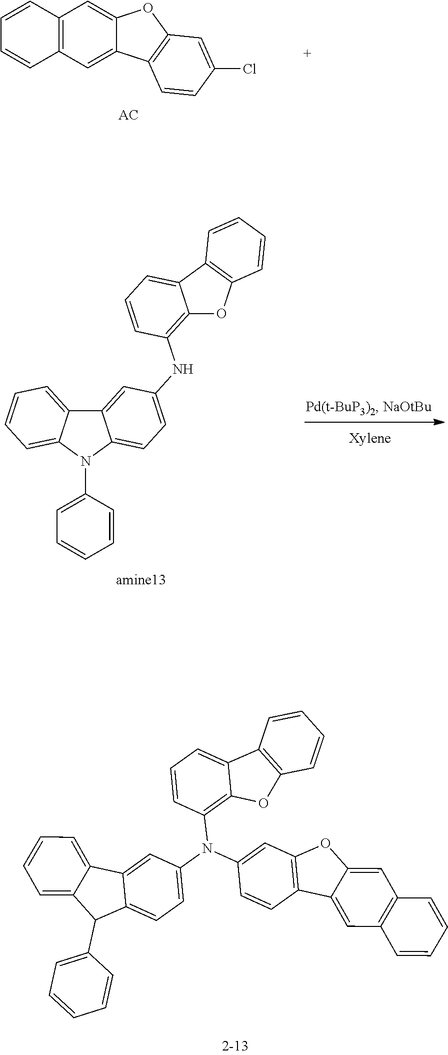 [SMILES: Clc1ccc2c(c1)oc1cc3ccccc3cc12 ; c1ccc(-n2c3ccccc3c3cc(Nc4cccc5c4oc4ccccc45)ccc32)cc1 ; c1ccc(C2c3ccccc3-c3cc(N(c4ccc5c(c4)oc4cc6ccccc6cc45)c4cccc5c4oc4ccccc45)ccc32)cc1]

Compound AC (10 g, 39.6 mmol), amine 13 (16.8 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.5 g of Compound 2-13. (Yield: 65%, MS: [M+H]+=641)

### Synthesis Example 2-14

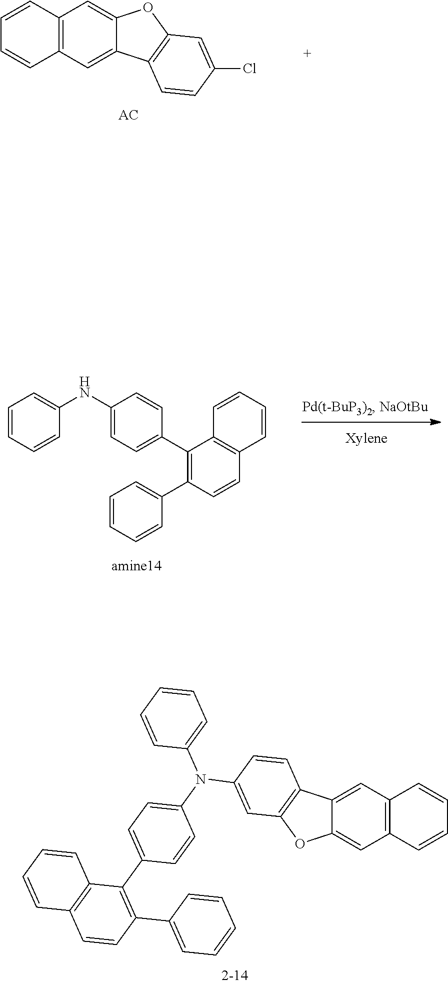 [SMILES: Clc1ccc2c(c1)oc1cc3ccccc3cc12 ; c1ccc(Nc2ccc(-c3c(-c4ccccc4)ccc4ccccc34)cc2)cc1 ; c1ccc(-c2ccc3ccccc3c2-c2ccc(N(c3ccccc3)c3ccc4c(c3)oc3cc5ccccc5cc34)cc2)cc1]

Compound AC (10 g, 39.6 mmol), amine 14 (14.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.9 g of Compound 2-14. (Yield: 64%, MS: [M+H]+=588)

### Synthesis Example 2-15

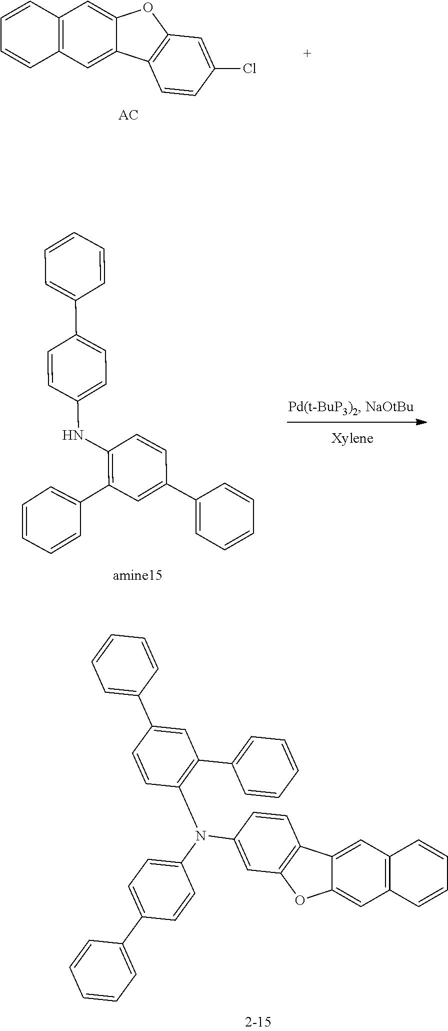 [SMILES: Clc1ccc2c(c1)oc1cc3ccccc3cc12 ; c1ccc(-c2ccc(Nc3ccc(-c4ccccc4)cc3-c3ccccc3)cc2)cc1 ; c1ccc(-c2ccc(N(c3ccc4c(c3)oc3cc5ccccc5cc34)c3ccc(-c4ccccc4)cc3-c3ccccc3)cc2)cc1]

Compound AC (10 g, 39.6 mmol), amine 15 (15.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound 2-15. (Yield: 60%, MS: [M+H]+=614)

### Synthesis Example 2-16

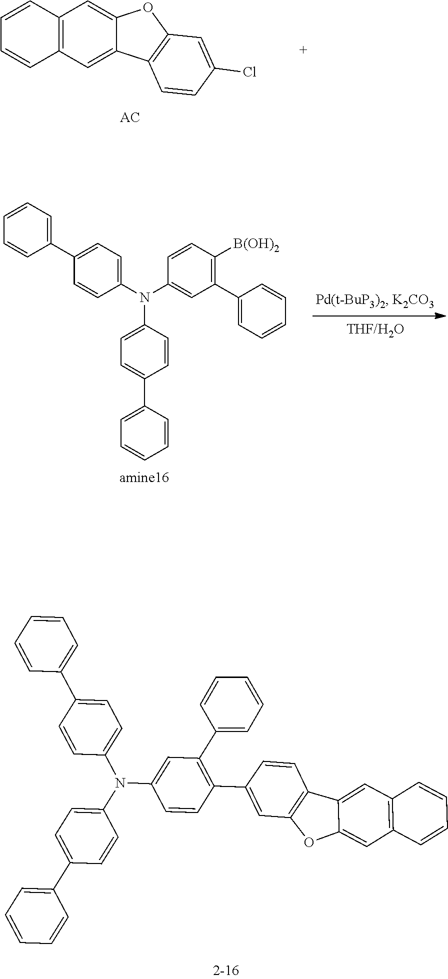 [SMILES: Clc1ccc2c(c1)oc1cc3ccccc3cc12 ; "AC" ; OB(O)c1ccc(N(c2ccc(-c3ccccc3)cc2)c2ccc(-c3ccccc3)cc2)cc1-c1ccccc1 ; c1ccc(-c2ccc(N(c3ccc(-c4ccccc4)cc3)c3ccc(-c4ccc5c(c4)oc4cc6ccccc6cc45)c(-c4ccccc4)c3)cc2)cc1]

Compound AC (15 g, 59.4 mmol) and amine 16 (32.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.2 g of Compound 2-16. (Yield: 69%, MS: [M+H]+=690)

### Synthesis Example 2-17

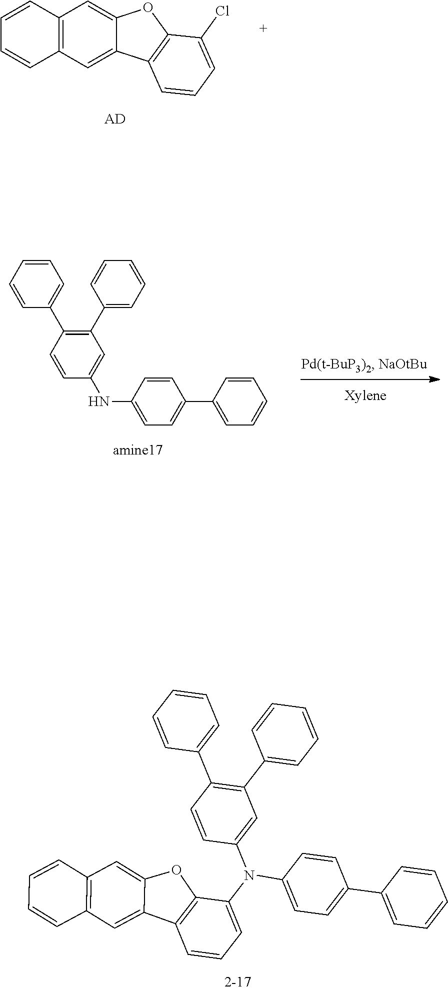 [SMILES: Clc1cccc2c1oc1cc3ccccc3cc12 ; "AD" ; c1ccc(-c2ccc(Nc3ccc(-c4ccccc4)c(-c4ccccc4)c3)cc2)cc1 ; c1ccc(-c2ccc(N(c3ccc(-c4ccccc4)c(-c4ccccc4)c3)c3cccc4c3oc3cc5ccccc5cc34)cc2)cc1]

Compound AD (10 g, 39.6 mmol), amine 17 (15.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.8 g of Compound 2-17. (Yield: 61%, MS: [M+H]+=614)

### Synthesis Example 2-18

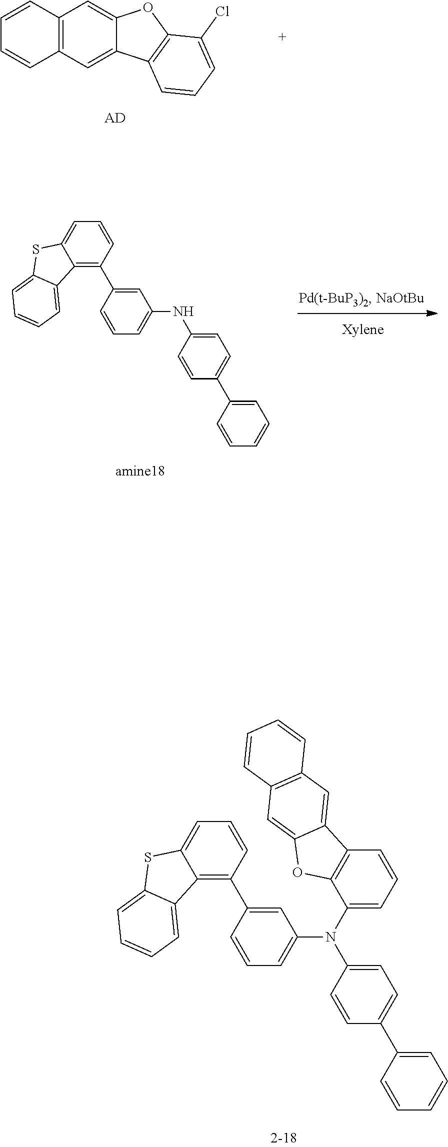 [SMILES: Clc1cccc2c1oc1cc3ccccc3cc12 ; "AD" ; c1ccc(-c2ccc(Nc3cccc(-c4cccc5sc6ccccc6c45)c3)cc2)cc1 ; c1ccc(-c2ccc(N(c3cccc(-c4cccc5sc6ccccc6c45)c3)c3cccc4c3oc3cc5ccccc5cc34)cc2)cc1]

Compound AD (10 g, 39.6 mmol), amine 18 (16.9 g 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.6 g of Compound 2-18. (Yield: 69%, MS: [M+H]+=644)

### Synthesis Example 2-19

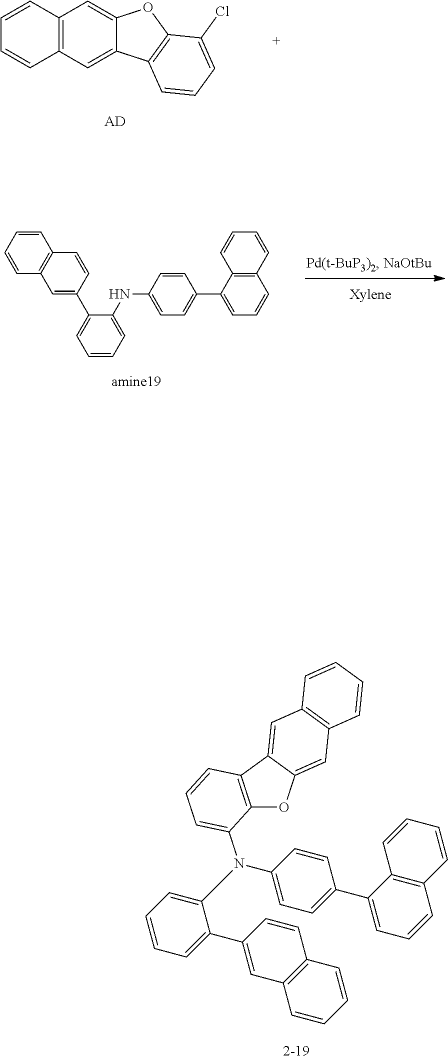 [SMILES: Clc1cccc2c1oc1cc3ccccc3cc12 ; "AD" ; c1ccc(-c2ccc3ccccc3c2)c(Nc2ccc(-c3cccc4ccccc34)cc2)c1 ; c1ccc(N(c2ccc(-c3cccc4ccccc34)cc2)c2cccc3c2oc2cc4ccccc4cc23)c(-c2ccc3ccccc3c2)c1]

Compound AD (10 g, 39.6 mmol), amine 19 (16.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.1 g of Compound 2-19. (Yield: 64%, MS: [M+H]+=638)

### Synthesis Example 2-20

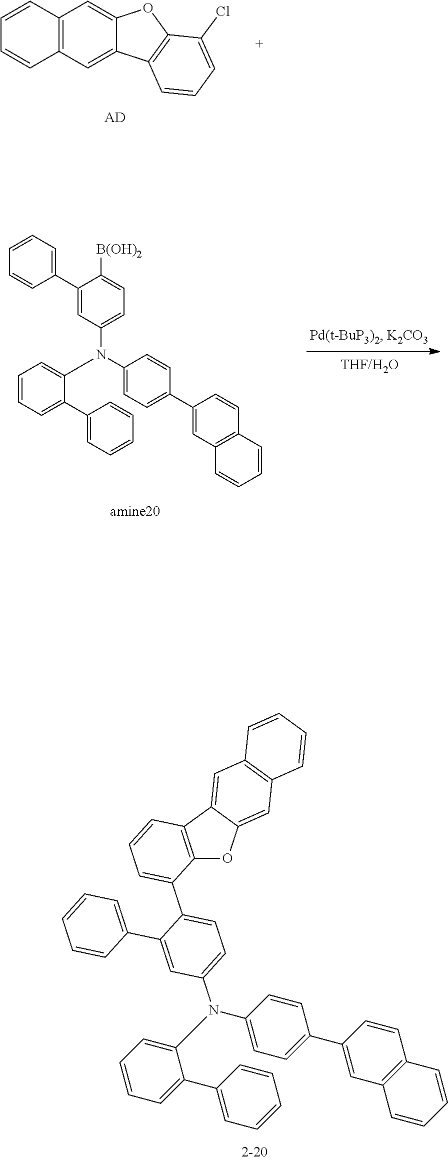 [SMILES: Clc1cccc2c1oc1cc3ccccc3cc12 ; "AD" ; OB(O)c1ccc(N(c2ccc(-c3ccc4ccccc4c3)cc2)c2ccccc2-c2ccccc2)cc1-c1ccccc1 ; c1ccc(-c2cc(N(c3ccc(-c4ccc5ccccc5c4)cc3)c3ccccc3-c3ccccc3)ccc2-c2cccc3c2oc2cc4ccccc4cc23)cc1]

Compound AD (15 g, 59.4 mmol) and amine 20 (35.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 32.9 g of Compound 2-20. (Yield: 75%, MS: [M+H]+=740)

### Synthesis Example 2-21

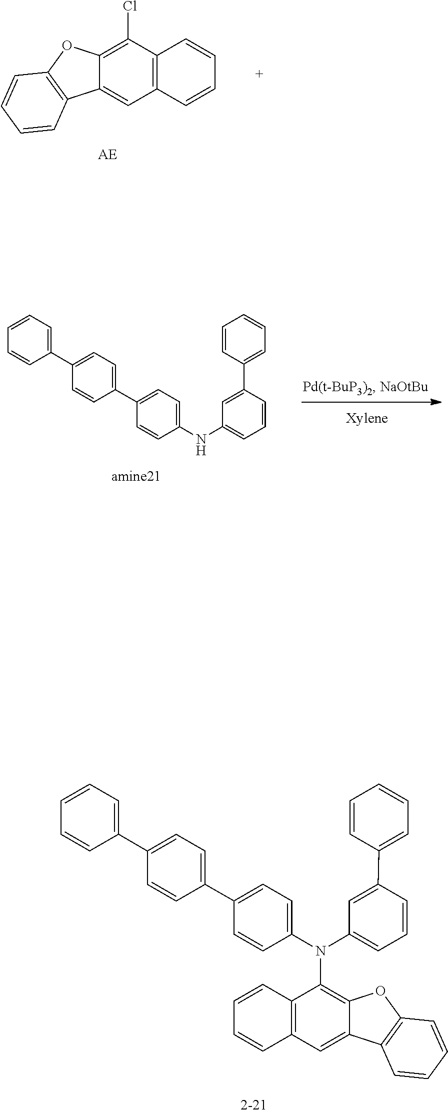 [SMILES: Clc1c2ccccc2cc2c1oc1ccccc12 ; c1ccc(-c2ccc(-c3ccc(Nc4cccc(-c5ccccc5)c4)cc3)cc2)cc1 ; c1ccc(-c2ccc(-c3ccc(N(c4cccc(-c5ccccc5)c4)c4c5ccccc5cc5c4oc4ccccc45)cc3)cc2)cc1]

Compound AE (10 g, 39.6 mmol), amine 21 (15.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.6 g of Compound 2-21. (Yield: 60%, MS: [M+H]+=614)

### Synthesis Example 2-22

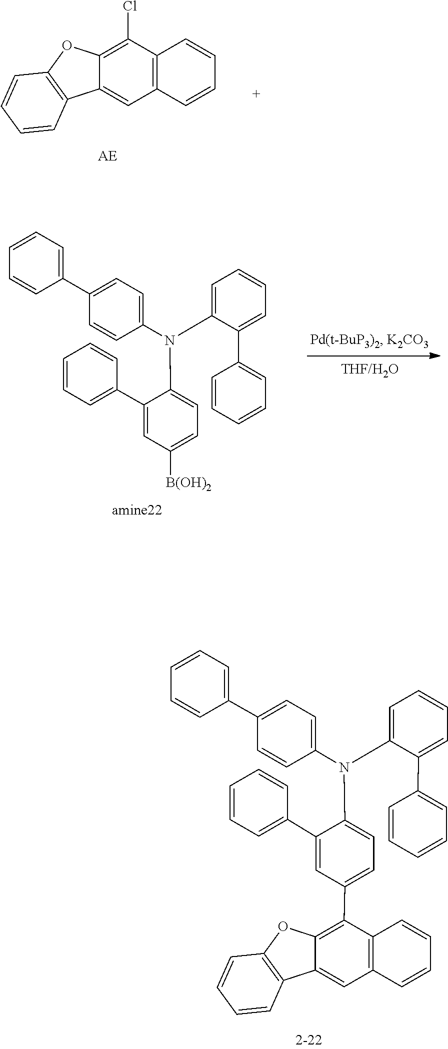 [SMILES: Clc1c2ccccc2cc2c1oc1ccccc12 ; OB(O)c1ccc(N(c2ccc(-c3ccccc3)cc2)c2ccccc2-c2ccccc2)c(-c2ccccc2)c1 ; c1ccc(-c2ccc(N(c3ccccc3-c3ccccc3)c3ccc(-c4c5ccccc5cc5c4oc4ccccc45)cc3-c3ccccc3)cc2)cc1]

Compound AE (15 g, 59.4 mmol) and amine 22 (32.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.3 g of Compound 2-22. (Yield: 74%, MS: [M+H]+=690)

### Synthesis Example 2-23

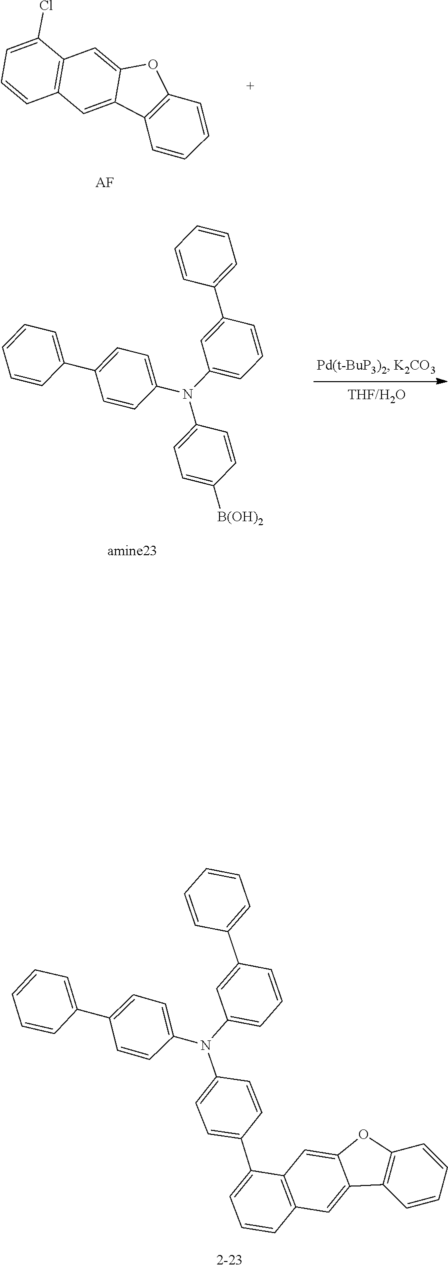 [SMILES: Clc1cccc2cc3c(cc12)oc1ccccc13 ; "AF" ; OB(O)c1ccc(N(c2ccc(-c3ccccc3)cc2)c2cccc(-c3ccccc3)c2)cc1 ; c1ccc(-c2ccc(N(c3ccc(-c4cccc5cc6c(cc45)oc4ccccc46)cc3)c3cccc(-c4ccccc4)c3)cc2)cc1]

Compound AF (15 g, 59.4 mmol) and amine 23 (27.5 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.9 g of Compound 2-23. (Yield: 74%, MS: [M+H]+=614)

### Synthesis Example 2-24

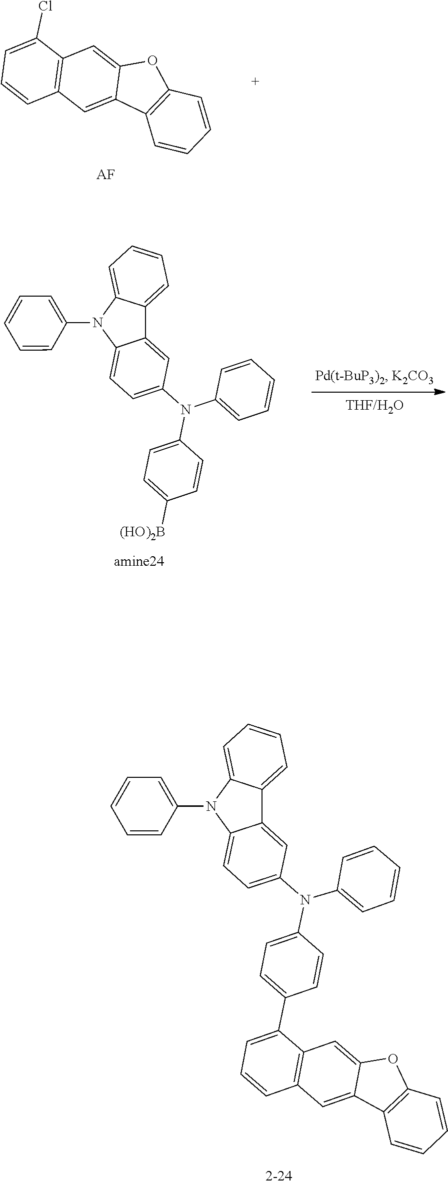 [SMILES: Clc1cccc2cc3c(cc12)oc1ccccc13 ; "AF" ; OB(O)c1ccc(N(c2ccccc2)c2ccc3c(c2)c2ccccc2n3-c2ccccc2)cc1 ; c1ccc(N(c2ccc(-c3cccc4cc5c(cc34)oc3ccccc35)cc2)c2ccc3c(c2)c2ccccc2n3-c2ccccc2)cc1]

Compound AF (15 g, 59.4 mmol) and amine 24 (28.3 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 2-24. (Yield: 71%, MS: [M+H]+=627)

### Synthesis Example 2-25

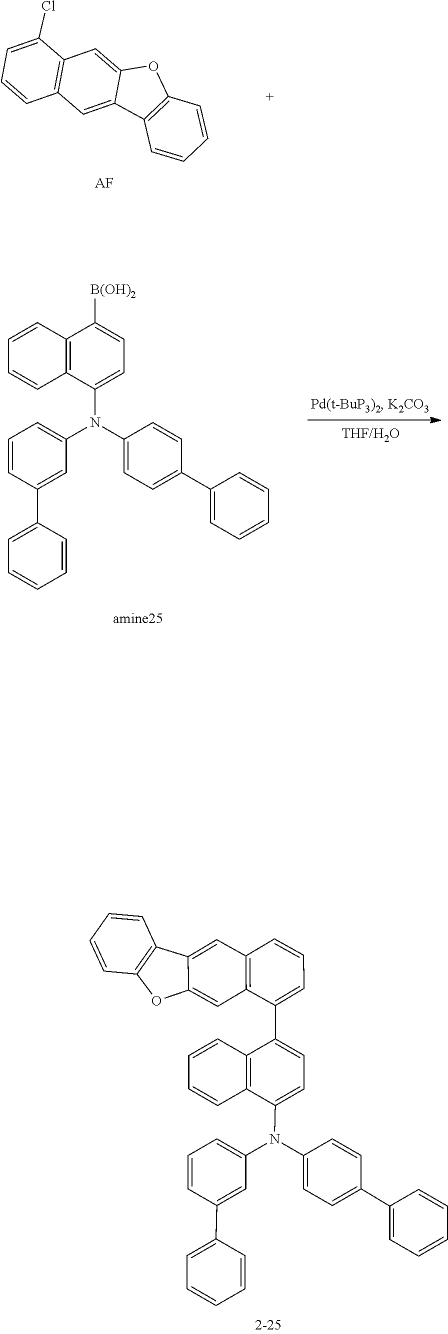 [SMILES: Clc1cccc2cc3c(cc12)oc1ccccc13 ; "AF" ; OB(O)c1ccc(N(c2ccc(-c3ccccc3)cc2)c2cccc(-c3ccccc3)c2)c2ccccc12 ; c1ccc(-c2ccc(N(c3cccc(-c4ccccc4)c3)c3ccc(-c4cccc5cc6c(cc45)oc4ccccc46)c4ccccc34)cc2)cc1]

Compound AF (15 g, 59.4 mmol) and amine 25 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.2 g of Compound 2-25. (Yield: 69%, MS: [M+H]+=664)

### Synthesis Example 2-26

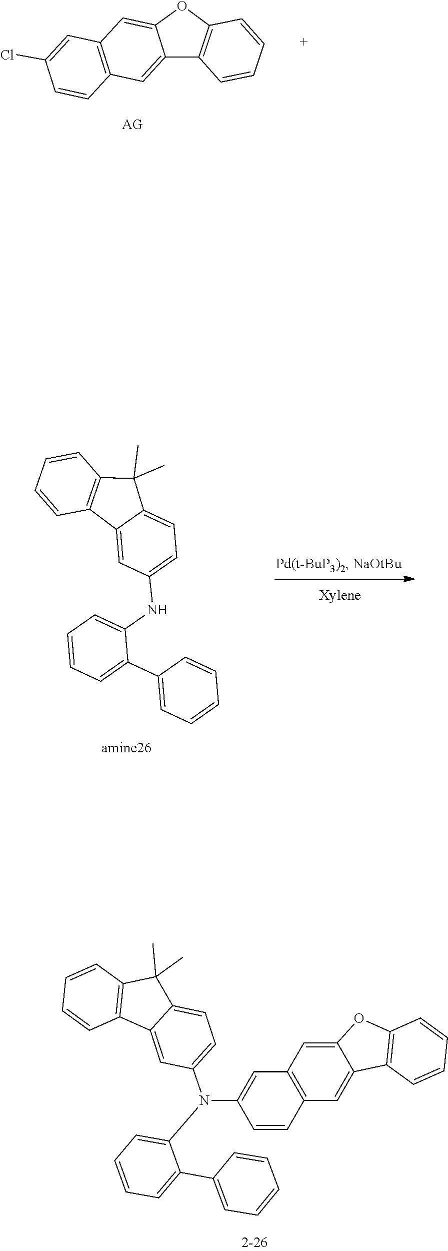 [SMILES: Clc1ccc2cc3c(cc2c1)oc1ccccc13 ; CC1(C)c2ccccc2-c2cc(Nc3ccccc3-c3ccccc3)ccc21 ; CC1(C)c2ccccc2-c2cc(N(c3ccc4cc5c(cc4c3)oc3ccccc35)c3ccccc3-c3ccccc3)ccc21]

Compound AG (10 g, 39.6 mmol), amine 26 (14.3 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.7 g of Compound 2-26. (Yield: 60%, MS: [M+H]+=578)

### Synthesis Example 2-27

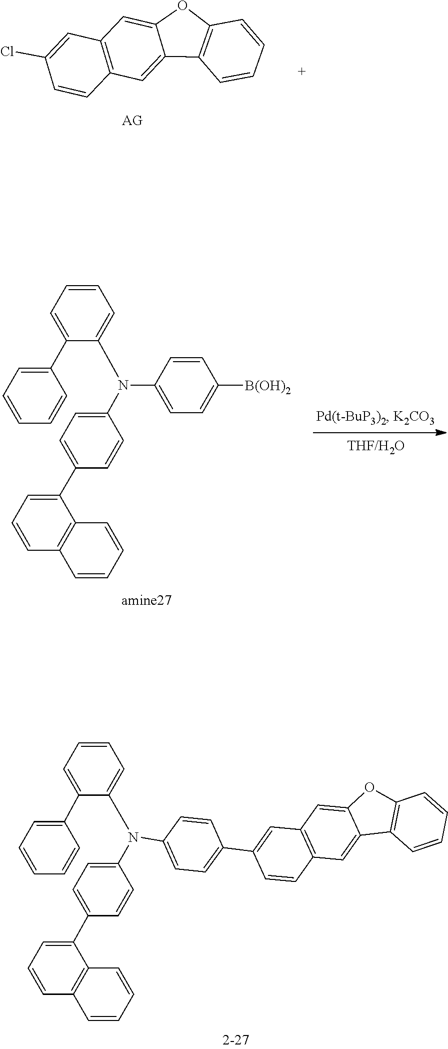 [SMILES: Clc1ccc2cc3c(cc2c1)oc1ccccc13 ; OB(O)c1ccc(N(c2ccc(-c3cccc4ccccc34)cc2)c2ccccc2-c2ccccc2)cc1 ; c1ccc(-c2ccccc2N(c2ccc(-c3ccc4cc5c(cc4c3)oc3ccccc35)cc2)c2ccc(-c3cccc4ccccc34)cc2)cc1]

Compound AG (15 g, 59.4 mmol) and amine 27 (30.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.2 g of Compound 2-27. (Yield: 69%, MS: [M+H]+=664)

### Synthesis Example 2-28

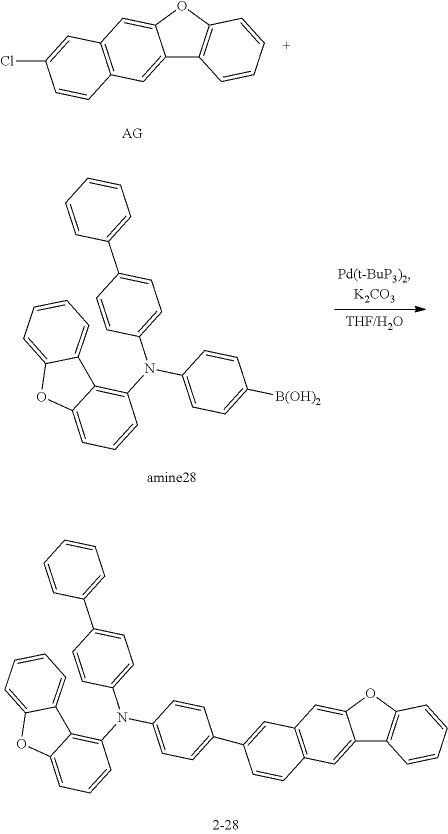 [SMILES: Clc1ccc2cc3c(cc2c1)oc1ccccc13 ; OB(O)c1ccc(N(c2ccc(-c3ccccc3)cc2)c2cccc3oc4ccccc4c23)cc1 ; c1ccc(-c2ccc(N(c3ccc(-c4ccc5cc6c(cc5c4)oc4ccccc46)cc3)c3cccc4oc5ccccc5c34)cc2)cc1]

Compound AG (15 g, 59.4 mmol) and amine 28 (28.4 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 24.2 g of Compound 2-28. (Yield: 65%, MS: [M+H]+=628)

### Synthesis Example 2-29

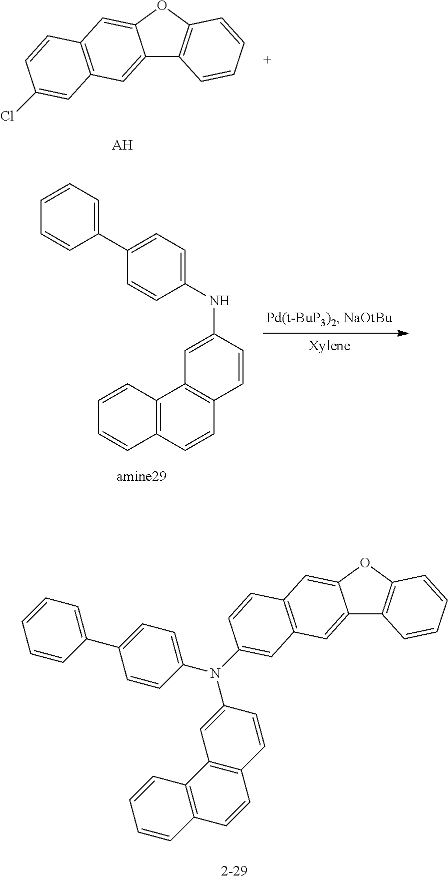 [SMILES: Clc1ccc2cc3oc4ccccc4c3cc2c1 ; "AH" ; c1ccc(-c2ccc(Nc3ccc4ccc5ccccc5c4c3)cc2)cc1 ; c1ccc(-c2ccc(N(c3ccc4cc5oc6ccccc6c5cc4c3)c3ccc4ccc5ccccc5c4c3)cc2)cc1]

Compound AH (10 g, 39.6 mmol), amine 29 (13.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-29. (Yield: 61%, MS: [M+H]+=562)

### Synthesis Example 2-30

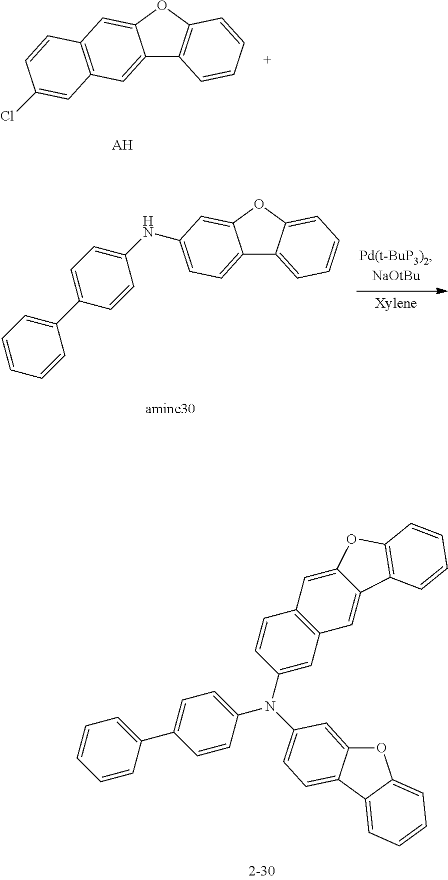 [SMILES: Clc1ccc2cc3oc4ccccc4c3cc2c1 ; "AH" ; c1ccc(-c2ccc(Nc3ccc4c(c3)oc3ccccc34)cc2)cc1 ; c1ccc(-c2ccc(N(c3ccc4cc5oc6ccccc6c5cc4c3)c3ccc4c(c3)oc3ccccc34)cc2)cc1]

Compound AH (10 g, 39.6 mmol), amine 30 (13.3 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.4 g of Compound 2-30. (Yield: 66%, MS: [M+H]+=552)

### Synthesis Example 2-31

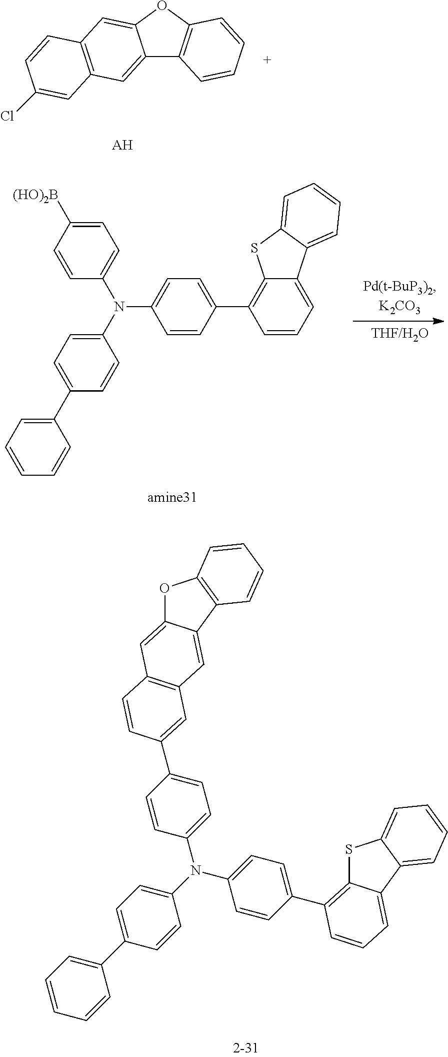 [SMILES: Clc1ccc2cc3oc4ccccc4c3cc2c1 ; "AH" ; OB(O)c1ccc(N(c2ccc(-c3ccccc3)cc2)c2ccc(-c3cccc4c3sc3ccccc34)cc2)cc1 ; c1ccc(-c2ccc(N(c3ccc(-c4ccc5cc6oc7ccccc7c6cc5c4)cc3)c3ccc(-c4cccc5c4sc4ccccc45)cc3)cc2)cc1]

Compound AH (15 g, 59.4 mmol) and amine 31 (34.1 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.5 g of Compound 2-31. (Yield: 69%, MS: [M+H]+=720)

### Synthesis Example 2-32

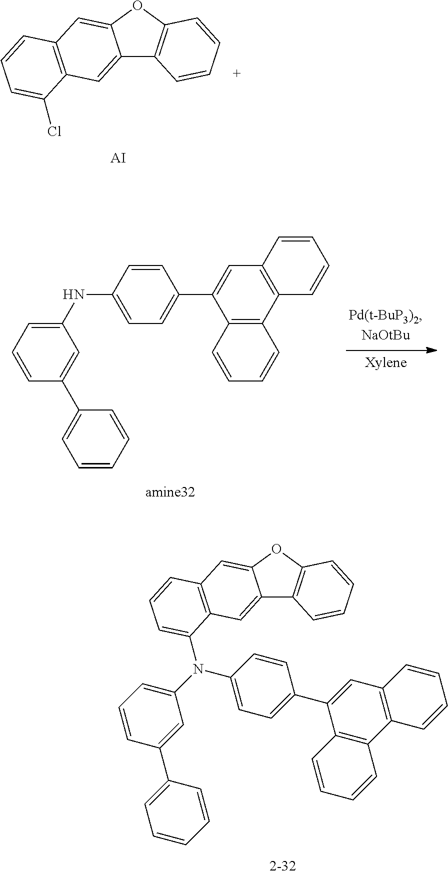 [SMILES: Clc1cccc2cc3oc4ccccc4c3cc12 ; "AI" ; c1ccc(-c2cccc(Nc3ccc(-c4cc5ccccc5c5ccccc45)cc3)c2)cc1 ; c1ccc(-c2cccc(N(c3ccc(-c4cc5ccccc5c5ccccc45)cc3)c3cccc4cc5oc6ccccc6c5cc34)c2)cc1]

Compound AI (10 g, 39.6 mmol), amine 32 (16.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 17.4 g of Compound 2-32. (Yield: 69%, MS: [M+H]+=638)

### Synthesis Example 2-33

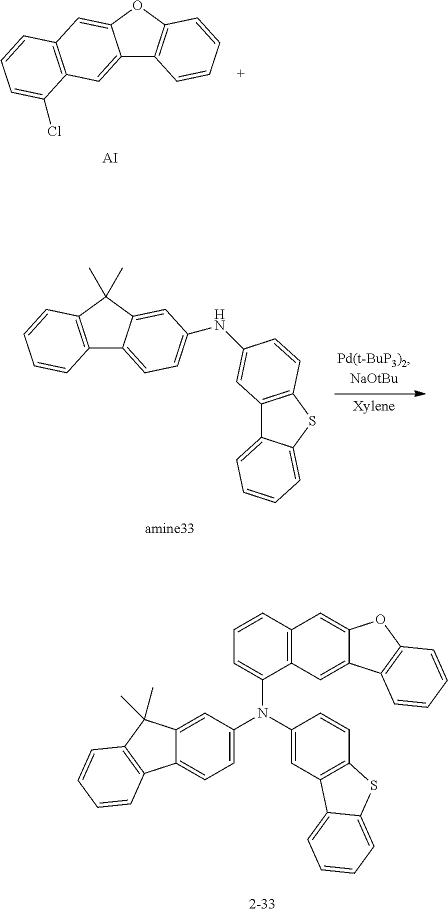 [SMILES: Clc1cccc2cc3oc4ccccc4c3cc12 ; "AI" ; CC1(C)c2ccccc2-c2ccc(Nc3ccc4sc5ccccc5c4c3)cc21 ; CC1(C)c2ccccc2-c2ccc(N(c3ccc4sc5ccccc5c4c3)c3cccc4cc5oc6ccccc6c5cc34)cc21]

Compound AI (10 g, 39.6 mmol), amine 33 (15.5 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.9 g of Compound 2-33. (Yield: 62%, MS: [M+H]+=608)

### Synthesis Example 2-34

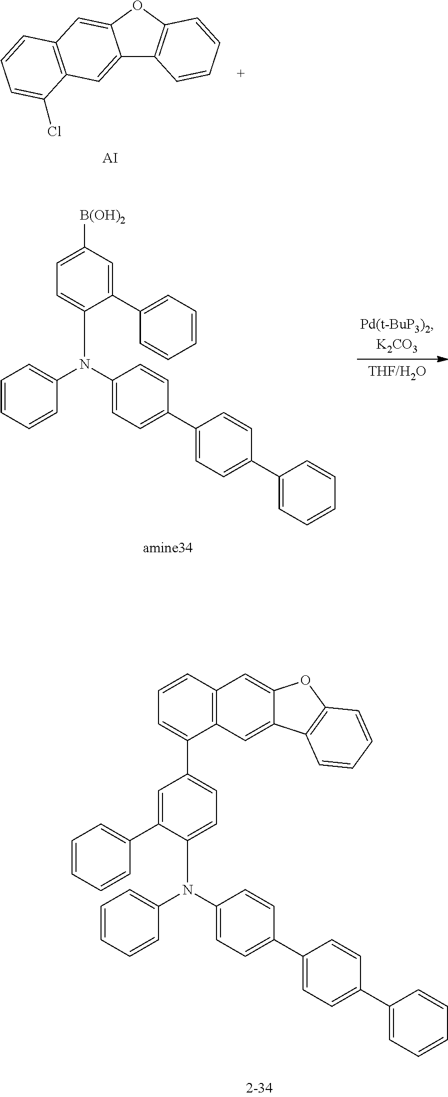 [SMILES: Clc1cccc2cc3oc4ccccc4c3cc12 ; "AI" ; OB(O)c1ccc(N(c2ccccc2)c2ccc(-c3ccc(-c4ccccc4)cc3)cc2)c(-c2ccccc2)c1 ; c1ccc(-c2ccc(-c3ccc(N(c4ccccc4)c4ccc(-c5cccc6cc7oc8ccccc8c7cc56)cc4-c4ccccc4)cc3)cc2)cc1]

Compound AI (15 g, 59.4 mmol) and amine 34 (3.6 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.4 g of Compound 2-34. (Yield: 67%, MS: [M+H]+=690)

### Synthesis Example 2-35

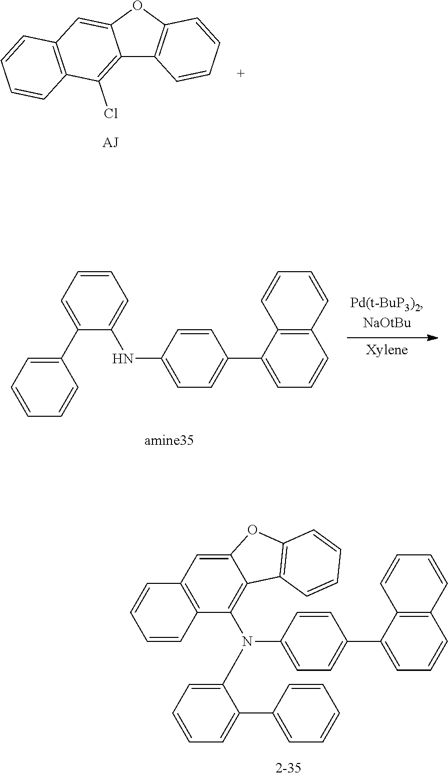

Compound AJ (10 g, 39.6 mmol), amine 35 (14.7 g, 39.6 mmol) and sodium tert-butoxide (12.6 g, 59.4 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.3 g of Compound 2-35. (Yield: 66%, MS: [M+H]+=588)

### Synthesis Example 2-36

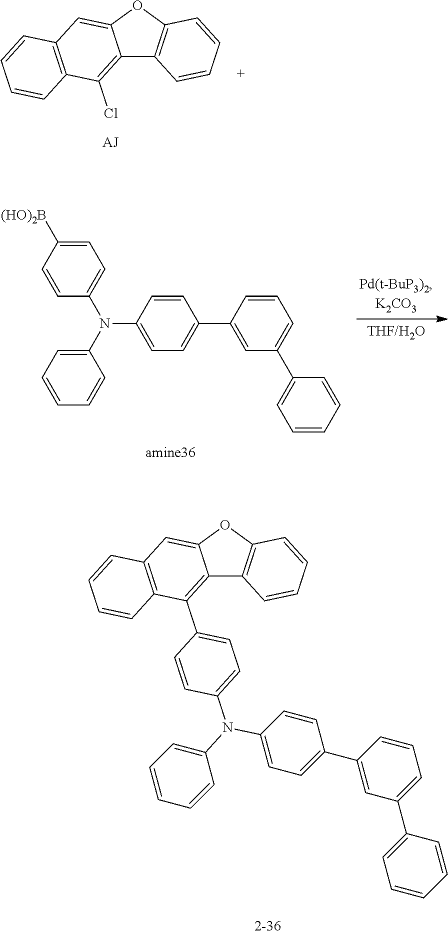

Compound AJ (15 g, 59.4 mmol) and amine 36 (27.5 g, 62.3 mmol) were added to 300 ml of THF, and the mixture was stirred and refluxed. Then, potassium carbonate (24.6 g, 178.1 mmol) was dissolved in 74 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.6 g of Compound 2-36. (Yield: 73%, MS: [M+H]+=614)

### Synthesis Example 2-37

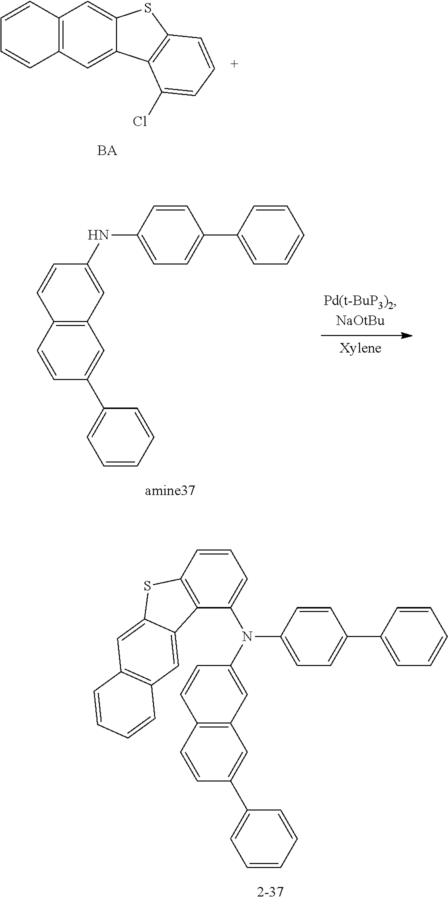 [SMILES: Clc1cccc2sc3cc4ccccc4cc3c12 ; c1ccc(-c2ccc(Nc3ccc4ccc(-c5ccccc5)cc4c3)cc2)cc1 ; c1ccc(-c2ccc(N(c3ccc4ccc(-c5ccccc5)cc4c3)c3cccc4sc5cc6ccccc6cc5c34)cc2)cc1]

Compound BA (10 g, 37.2 mmol), amine 37 (13.8 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.9 g of Compound 2-37. (Yield: 62%, MS: [M+H]+=604)

### Synthesis Example 2-38

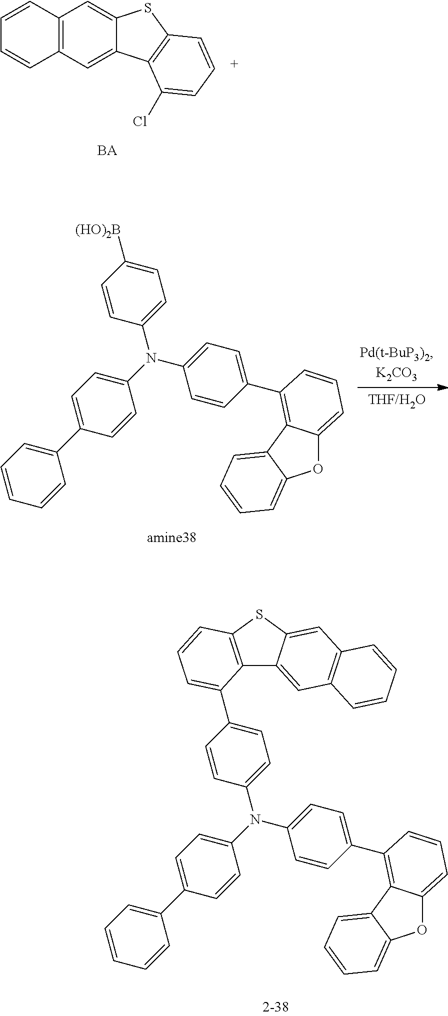 [SMILES: Clc1cccc2sc3cc4ccccc4cc3c12 ; OB(O)c1ccc(N(c2ccc(-c3ccccc3)cc2)c2ccc(-c3cccc4oc5ccccc5c34)cc2)cc1 ; c1ccc(-c2ccc(N(c3ccc(-c4cccc5oc6ccccc6c45)cc3)c3ccc(-c4cccc5sc6cc7ccccc7cc6c45)cc3)cc2)cc1]

Compound BA (15 g, 55.8 mmol) and amine 38 (31.1 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.5 g of Compound 2-38. (Yield: 66%, MS: [M+H]+=720)

### Synthesis Example 2-39

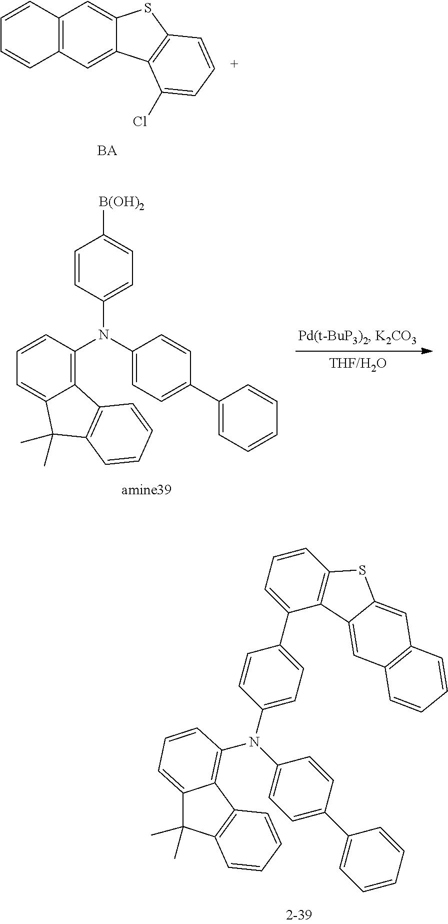 [SMILES: Clc1cccc2sc3cc4ccccc4cc3c12 ; CC1(C)c2ccccc2-c2c(N(c3ccc(B(O)O)cc3)c3ccc(-c4ccccc4)cc3)cccc21 ; CC1(C)c2ccccc2-c2c(N(c3ccc(-c4ccccc4)cc3)c3ccc(-c4cccc5sc6cc7ccccc7cc6c45)cc3)cccc21]

Compound BA (15 g, 55.8 mmol) and amine 39 (28.2 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.5 g of Compound 2-39. (Yield: 71%, MS: [M+H]+=670)

### Synthesis Example 2-40

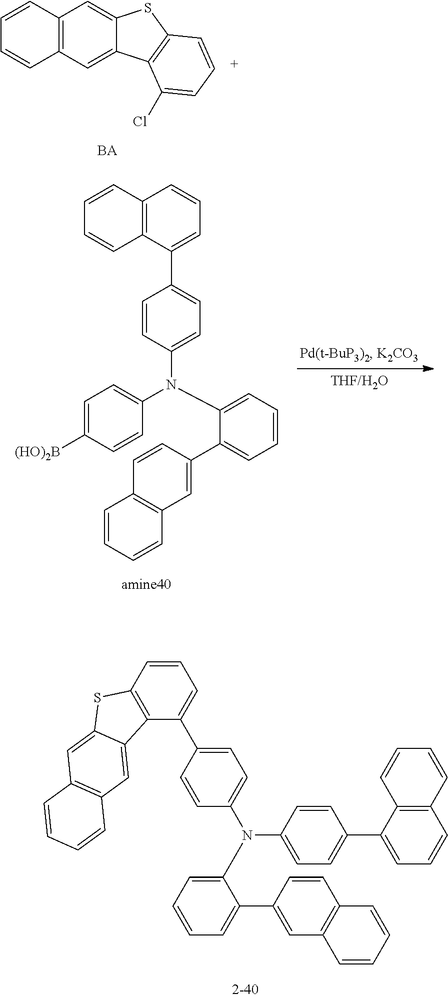 [SMILES: Clc1cccc2sc3cc4ccccc4cc3c12 ; OB(O)c1ccc(N(c2ccc(-c3cccc4ccccc34)cc2)c2ccccc2-c2ccc3ccccc3c2)cc1 ; c1ccc(N(c2ccc(-c3cccc4ccccc34)cc2)c2ccc(-c3cccc4sc5cc6ccccc6cc5c34)cc2)c(-c2ccc3ccccc3c2)c1]

Compound BA (15 g, 55.8 mmol) and amine 40 (31.7 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.1 g of Compound 2-40. (Yield: 69%, MS: [M+H]+=730)

### Synthesis Example 2-41

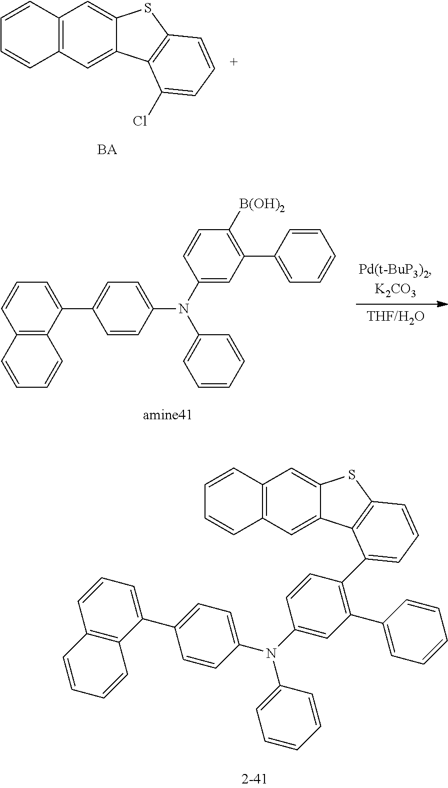 [SMILES: Clc1cccc2sc3cc4ccccc4cc3c12 ; OB(O)c1ccc(N(c2ccccc2)c2ccc(-c3cccc4ccccc34)cc2)cc1-c1ccccc1 ; c1ccc(-c2cc(N(c3ccccc3)c3ccc(-c4cccc5ccccc45)cc3)ccc2-c2cccc3sc4cc5ccccc5cc4c23)cc1]

Compound BA (15 g, 55.8 mmol) and amine 41 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 2-41. (Yield: 67%, MS: [M+H]+=680)

### Synthesis Example 2-42

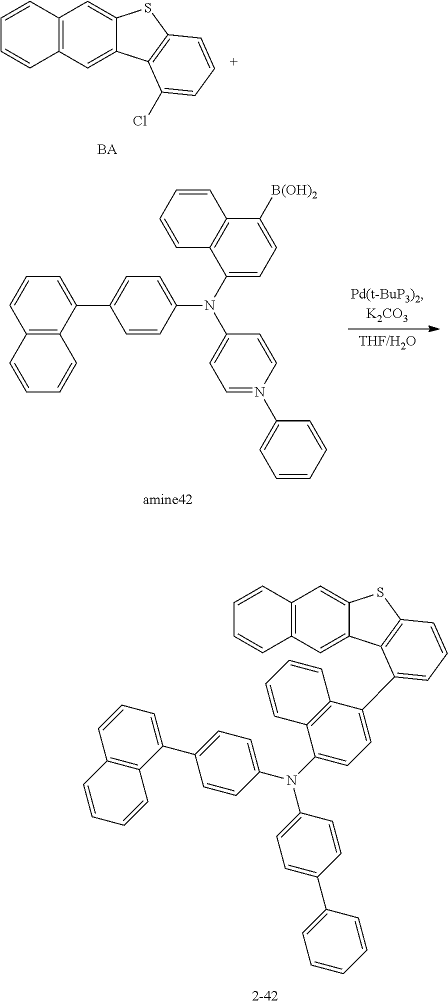

Compound BA (15 g, 55.8 mmol) and amine 42 (31.7 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 29.3 g of Compound 2-42. (Yield: 72%, MS: [M+H]+=730)

### Synthesis Example 2-43

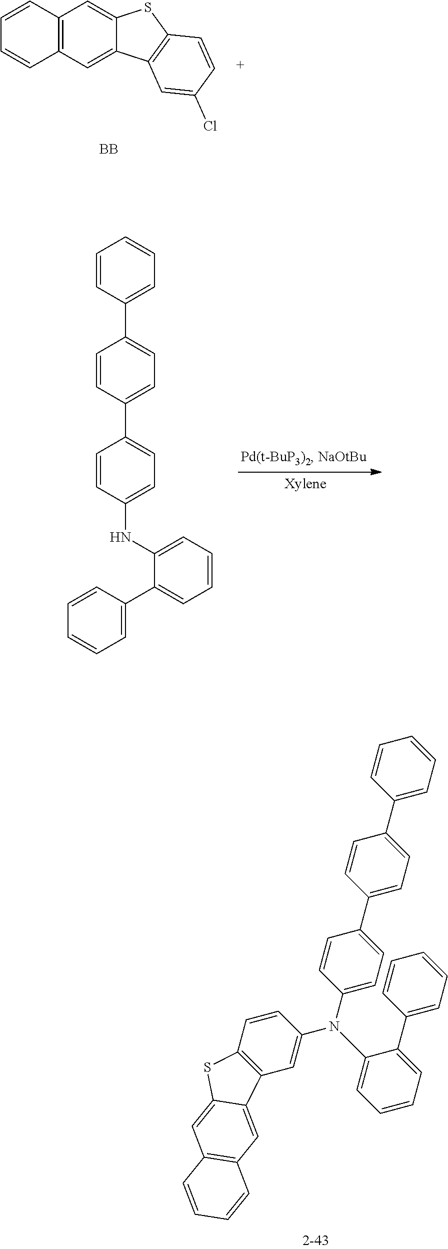 [SMILES: Clc1ccc2sc3cc4ccccc4cc3c2c1 ; "BB" ; c1ccc(-c2ccc(-c3ccc(Nc4ccccc4-c4ccccc4)cc3)cc2)cc1 ; c1ccc(-c2ccc(-c3ccc(N(c4ccc5sc6cc7ccccc7cc6c5c4)c4ccccc4-c4ccccc4)cc3)cc2)cc1]

Compound BB (10 g, 37.2 mmol), amine 43 (14.8 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.3 g of Compound 2-43. (Yield: 61%, MS: [M+H]+=630)

### Synthesis Example 2-44

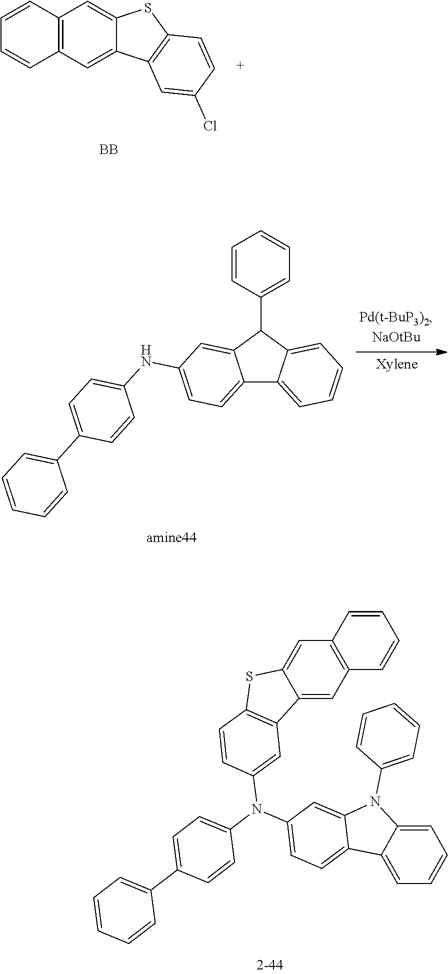 [SMILES: Clc1ccc2sc3cc4ccccc4cc3c2c1 ; "BB" ; c1ccc(-c2ccc(Nc3ccc4c(c3)C(c3ccccc3)c3ccccc3-4)cc2)cc1 ; c1ccc(-c2ccc(N(c3ccc4sc5cc6ccccc6cc5c4c3)c3ccc4c5ccccc5n(-c5ccccc5)c4c3)cc2)cc1]

Compound BB (10 g, 37.2 mmol), amine 44 (15.3 g 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.5 g of Compound 2-44. (Yield: 65%, MS: [M+H]+=643)

### Synthesis Example 2-45

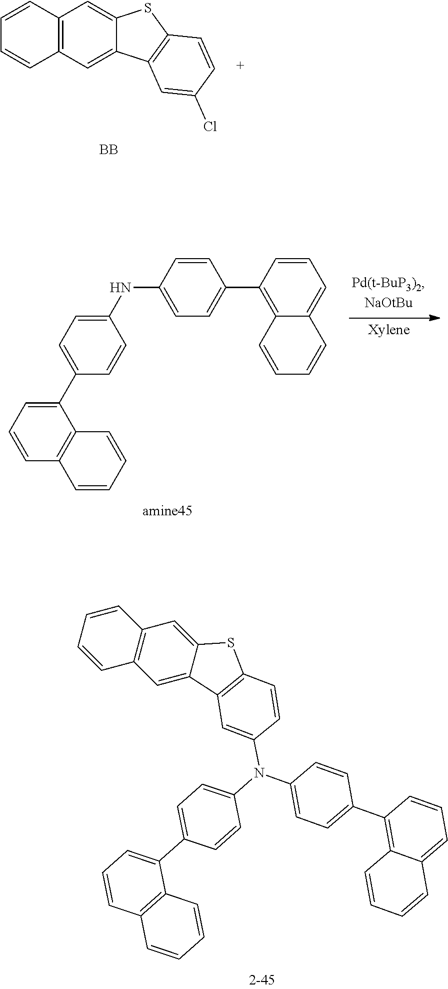 [SMILES: Clc1ccc2sc3cc4ccccc4cc3c2c1 ; "BB" ; c1ccc2c(-c3ccc(Nc4ccc(-c5cccc6ccccc56)cc4)cc3)cccc2c1 ; c1ccc2cc3c(cc2c1)sc1ccc(N(c2ccc(-c4cccc5ccccc45)cc2)c2ccc(-c4cccc5ccccc45)cc2)cc13]

Compound BB (10 g, 37.2 mmol), amine 45 (15.7 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.3 g of Compound 2-45. (Yield: 63%, MS: [M+H]+=654)

### Synthesis Example 2-46

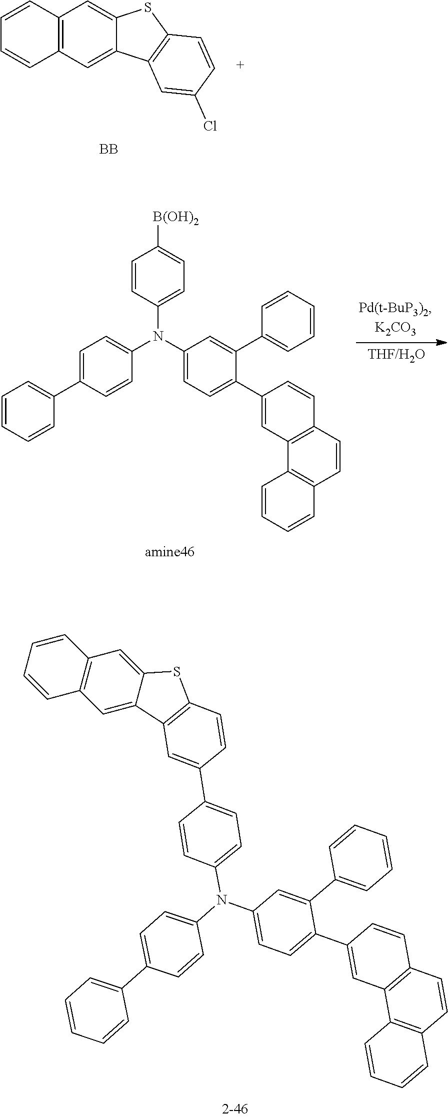 [SMILES: Clc1ccc2sc3cc4ccccc4cc3c2c1 ; "BB" ; OB(O)c1ccc(N(c2ccc(-c3ccccc3)cc2)c2ccc(-c3ccc4ccc5ccccc5c4c3)c(-c3ccccc3)c2)cc1 ; c1ccc(-c2ccc(N(c3ccc(-c4ccc5sc6cc7ccccc7cc6c5c4)cc3)c3ccc(-c4ccc5ccc6ccccc6c5c4)c(-c4ccccc4)c3)cc2)cc1]

Compound BB (15 g, 55.8 mmol) and amine 46 (36.2 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 31 g of Compound 2-46. (Yield: 69%, MS: [M+H]+=806)

### Synthesis Example 2-47

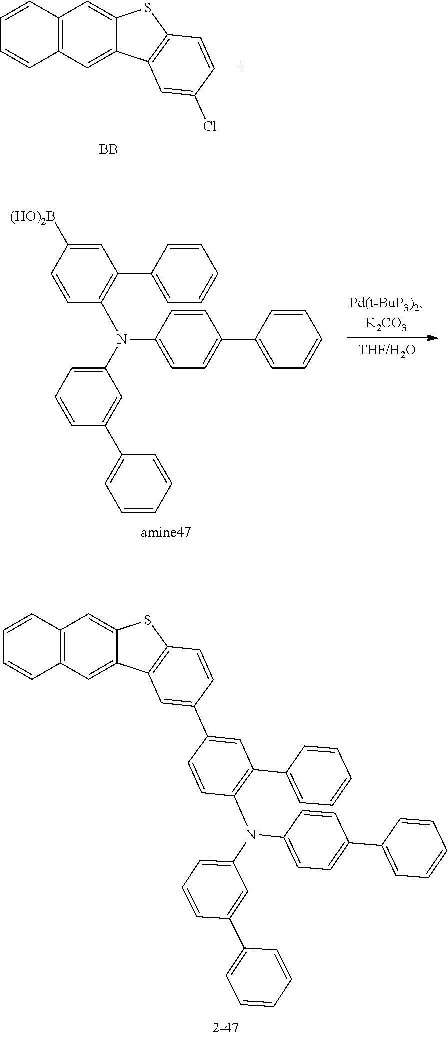 [SMILES: Clc1ccc2sc3cc4ccccc4cc3c2c1 ; "BB" ; OB(O)c1ccc(N(c2ccc(-c3ccccc3)cc2)c2cccc(-c3ccccc3)c2)c(-c2ccccc2)c1 ; c1ccc(-c2ccc(N(c3cccc(-c4ccccc4)c3)c3ccc(-c4ccc5sc6cc7ccccc7cc6c5c4)cc3-c3ccccc3)cc2)cc1]

Compound BB (15 g, 55.8 mmol) and amine 47 (30.3 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.4 g of Compound 2-47. (Yield: 67%, MS: [M+H]+=706)

### Synthesis Example 2-48

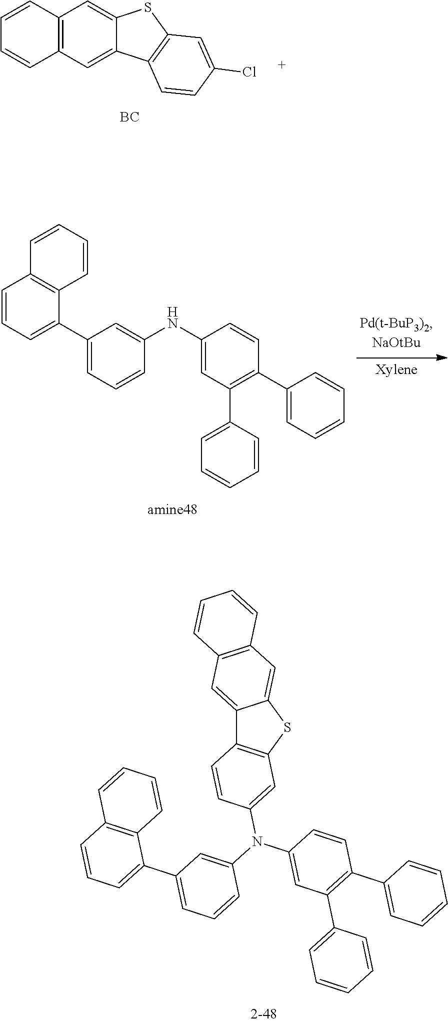 [SMILES: Clc1ccc2c(c1)sc1cc3ccccc3cc12 ; "BC" ; c1ccc(-c2ccc(Nc3cccc(-c4cccc5ccccc45)c3)cc2-c2ccccc2)cc1 ; c1ccc(-c2ccc(N(c3cccc(-c4cccc5ccccc45)c3)c3ccc4c(c3)sc3cc5ccccc5cc34)cc2-c2ccccc2)cc1]

Compound BC (10 g, 37.2 mmol), amine 48 (16.7 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.2 g of Compound 2-48. (Yield: 64%, MS: [M+H]+=680)

### Synthesis Example 2-49

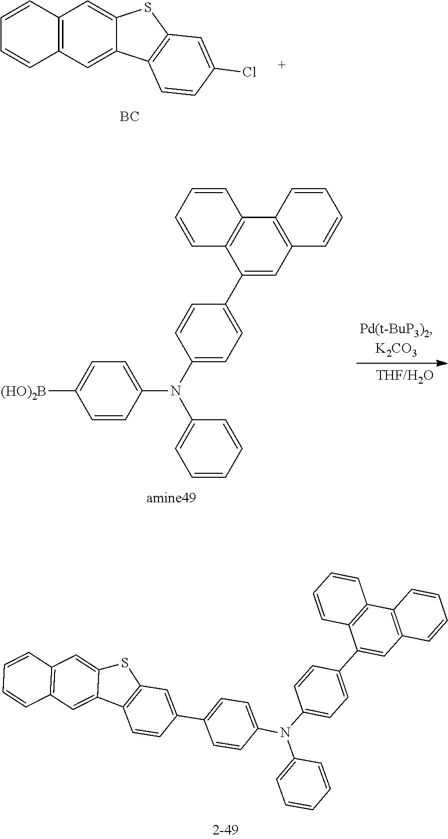 [SMILES: Clc1ccc2c(c1)sc1cc3ccccc3cc12 ; "BC" ; OB(O)c1ccc(N(c2ccccc2)c2ccc(-c3cc4ccccc4c4ccccc34)cc2)cc1 ; c1ccc(N(c2ccc(-c3ccc4c(c3)sc3cc5ccccc5cc34)cc2)c2ccc(-c3cc4ccccc4c4ccccc34)cc2)cc1]

Compound BC (15 g, 55.8 mmol) and amine 49 (27.3 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.7 g of Compound 2-49. (Yield: 65%, MS: [M+H]+=654)

### Synthesis Example 2-50

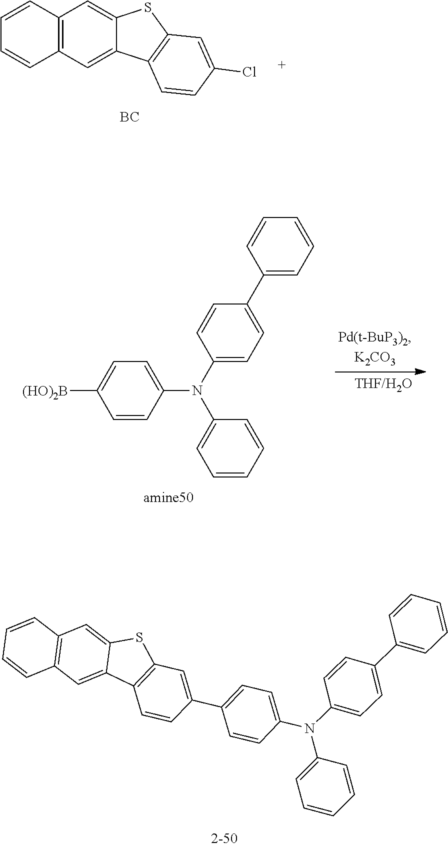 [SMILES: Clc1ccc2c(c1)sc1cc3ccccc3cc12 ; "BC" ; OB(O)c1ccc(N(c2ccccc2)c2ccc(-c3ccccc3)cc2)cc1 ; c1ccc(-c2ccc(N(c3ccccc3)c3ccc(-c4ccc5c(c4)sc4cc6ccccc6cc45)cc3)cc2)cc1]

Compound BC (15 g, 55.8 mmol) and amine 50 (21.4 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.8 g of Compound 2-50. (Yield: 74%, MS: [M+H]+=554)

### Synthesis Example 2-51

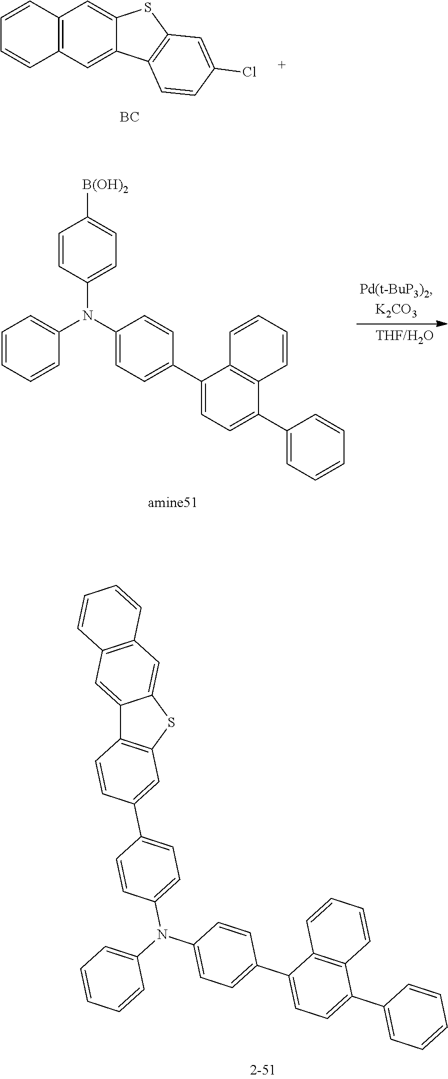 [SMILES: Clc1ccc2c(c1)sc1cc3ccccc3cc12 ; "BC" ; OB(O)c1ccc(N(c2ccccc2)c2ccc(-c3ccc(-c4ccccc4)c4ccccc34)cc2)cc1 ; c1ccc(-c2ccc(-c3ccc(N(c4ccccc4)c4ccc(-c5ccc6c(c5)sc5cc7ccccc7cc56)cc4)cc3)c3ccccc23)cc1]

Compound BC (15 g, 55.8 mmol) and amine 51 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.1 g of Compound 2-51. (Yield: 74%, MS: [M+H]+=680)

### Synthesis Example 2-52

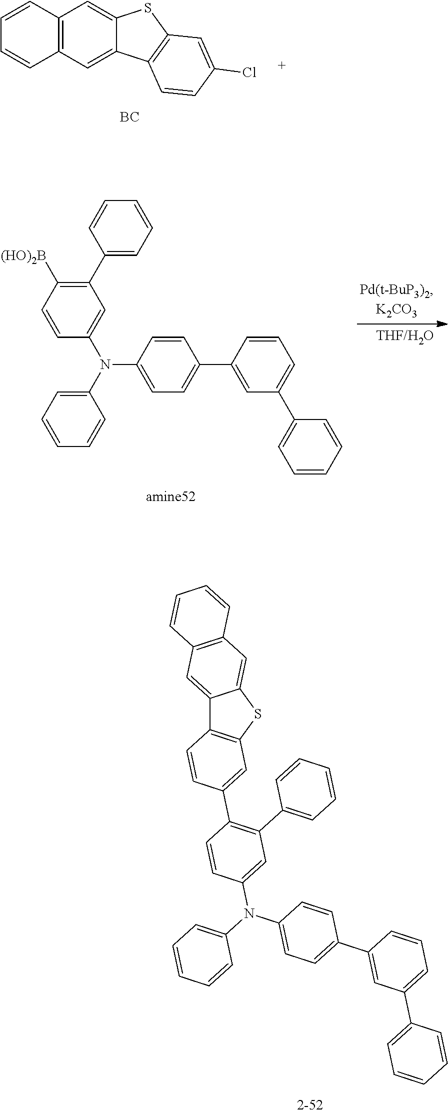 [SMILES: Clc1ccc2c(c1)sc1cc3ccccc3cc12 ; "BC" ; OB(O)c1ccc(N(c2ccccc2)c2ccc(-c3cccc(-c4ccccc4)c3)cc2)cc1-c1ccccc1 ; c1ccc(-c2cccc(-c3ccc(N(c4ccccc4)c4ccc(-c5ccc6c(c5)sc5cc7ccccc7cc56)c(-c5ccccc5)c4)cc3)c2)cc1]

Compound BC (15 g, 55.8 mmol) and amine 52 (30.3 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 2-52. (Yield: 66%, MS: [M+H]+=706)

### Synthesis Example 2-53

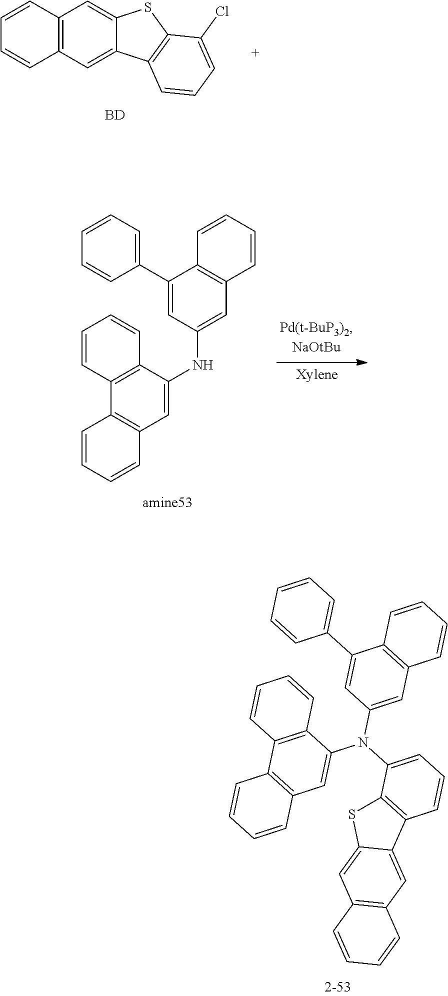 [SMILES: Clc1cccc2c1sc1cc3ccccc3cc12 ; "BD" ; c1ccc(-c2cc(Nc3cc4ccccc4c4ccccc34)cc3ccccc23)cc1 ; c1ccc(-c2cc(N(c3cc4ccccc4c4ccccc34)c3cccc4c3sc3cc5ccccc5cc34)cc3ccccc23)cc1]

Compound BD (10 g, 37.2 mmol), amine 53 (14.7 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 14.7 g of Compound 2-53. (Yield: 63%, MS: [M+H]+=628)

### Synthesis Example 2-54

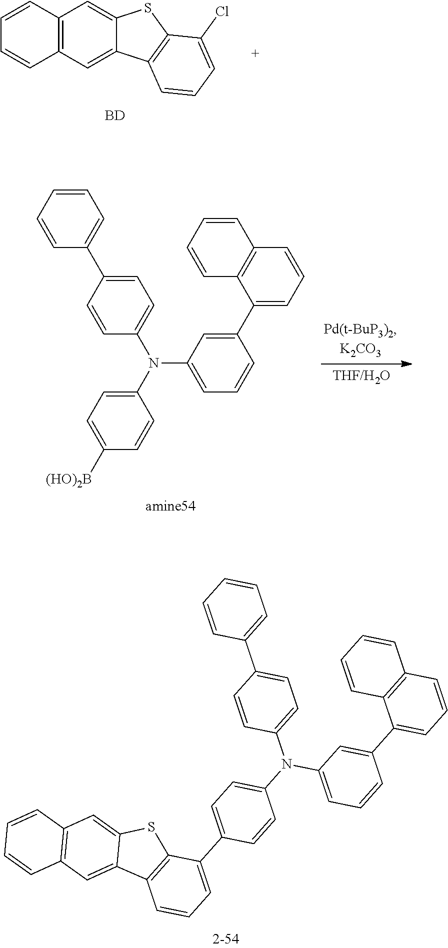 [SMILES: Clc1cccc2c1sc1cc3ccccc3cc12 ; "BD" ; OB(O)c1ccc(N(c2ccc(-c3ccccc3)cc2)c2cccc(-c3cccc4ccccc34)c2)cc1 ; c1ccc(-c2ccc(N(c3ccc(-c4cccc5c4sc4cc6ccccc6cc45)cc3)c3cccc(-c4cccc5ccccc45)c3)cc2)cc1]

Compound BD (15 g, 55.8 mmol) and amine 54 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26.9 g of Compound 2-54. (Yield: 71%, MS: [M+H]+=680)

### Synthesis Example 2-55

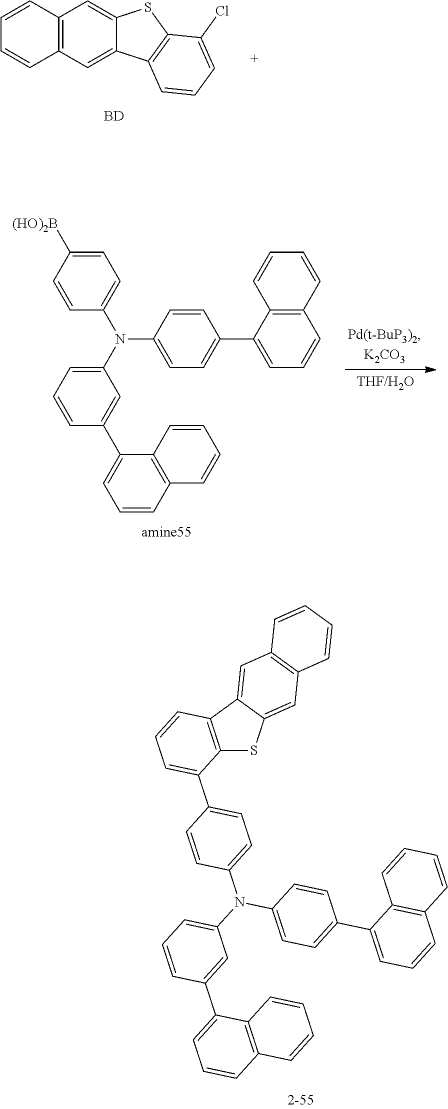 [SMILES: Clc1cccc2c1sc1cc3ccccc3cc12 ; "BD" ; OB(O)c1ccc(N(c2ccc(-c3cccc4ccccc34)cc2)c2cccc(-c3cccc4ccccc34)c2)cc1 ; c1cc(-c2cccc3ccccc23)cc(N(c2ccc(-c3cccc4ccccc34)cc2)c2ccc(-c3cccc4c3sc3cc5ccccc5cc34)cc2)c1]

Compound BD (15 g, 55.8 mmol) and amine 55 (31.7 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.1 g of Compound 2-55. (Yield: 74%, MS: [M+H]+=730)

### Synthesis Example 2-56

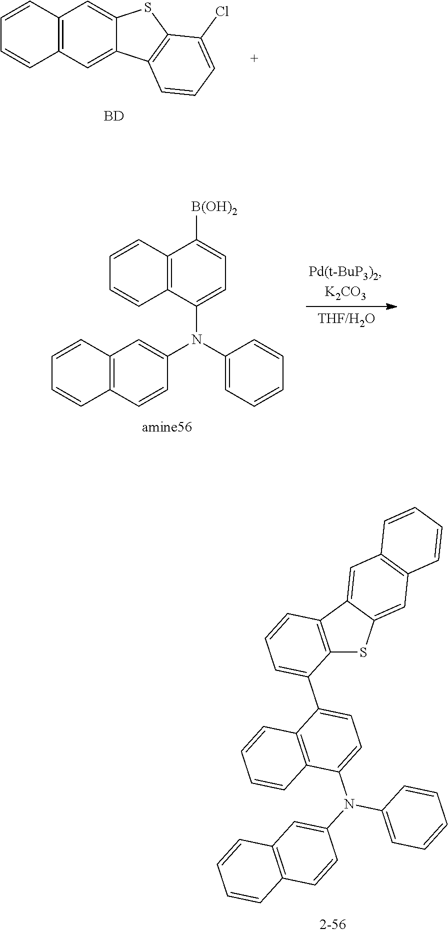 [SMILES: Clc1cccc2c1sc1cc3ccccc3cc12 ; "BD" ; OB(O)c1ccc(N(c2ccccc2)c2ccc3ccccc3c2)c2ccccc12 ; c1ccc(N(c2ccc3ccccc3c2)c2ccc(-c3cccc4c3sc3cc5ccccc5cc34)c3ccccc23)cc1]

Compound BD (15 g, 55.8 mmol) and amine 56 (22.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 22.9 g of Compound 2-56. (Yield: 71%, MS: [M+H]+=578)

### Synthesis Example 2-57

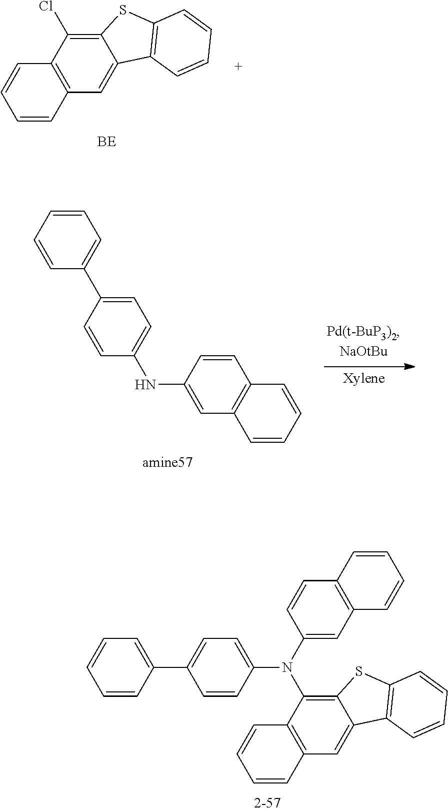 [SMILES: Clc1c2ccccc2cc2c1sc1ccccc12 ; c1ccc(-c2ccc(Nc3ccc4ccccc4c3)cc2)cc1 ; c1ccc(-c2ccc(N(c3ccc4ccccc4c3)c3c4ccccc4cc4c3sc3ccccc34)cc2)cc1]

Compound BE (10 g, 37.2 mmol), amine 57 (11 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-57. (Yield: 69%, MS: [M+H]+=528)

### Synthesis Example 2-58

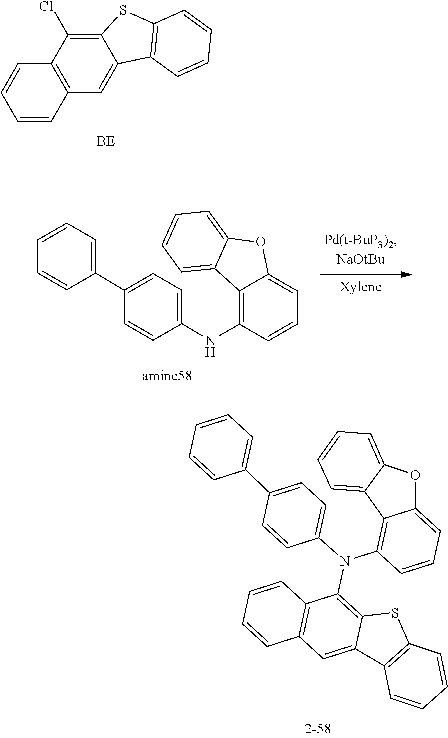 [SMILES: Clc1c2ccccc2cc2c1sc1ccccc12 ; c1ccc(-c2ccc(Nc3cccc4oc5ccccc5c34)cc2)cc1 ; c1ccc(-c2ccc(N(c3c4ccccc4cc4c3sc3ccccc34)c3cccc4oc5ccccc5c34)cc2)cc1]

Compound BE (10 g, 37.2 mmol), amine 58 (12.5 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.5 g of Compound 2-58. (Yield: 64%, MS: [M+H]+=568)

### Synthesis Example 2-59

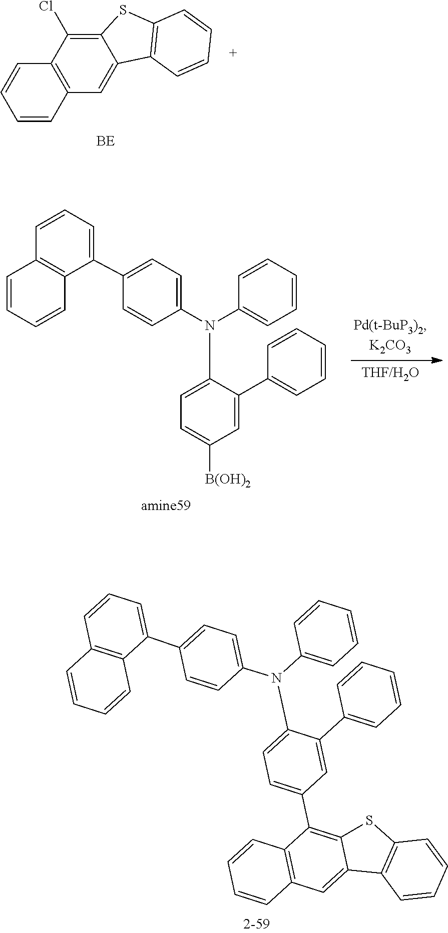 [SMILES: Clc1c2ccccc2cc2c1sc1ccccc12 ; OB(O)c1ccc(N(c2ccccc2)c2ccc(-c3cccc4ccccc34)cc2)c(-c2ccccc2)c1 ; c1ccc(-c2cc(-c3c4ccccc4cc4c3sc3ccccc34)ccc2N(c2ccccc2)c2ccc(-c3cccc4ccccc34)cc2)cc1]

Compound BE (15 g, 55.8 mmol) and amine 59 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 2-59. (Yield: 67%, MS: [M+H]+=680)

### Synthesis Example 2-60

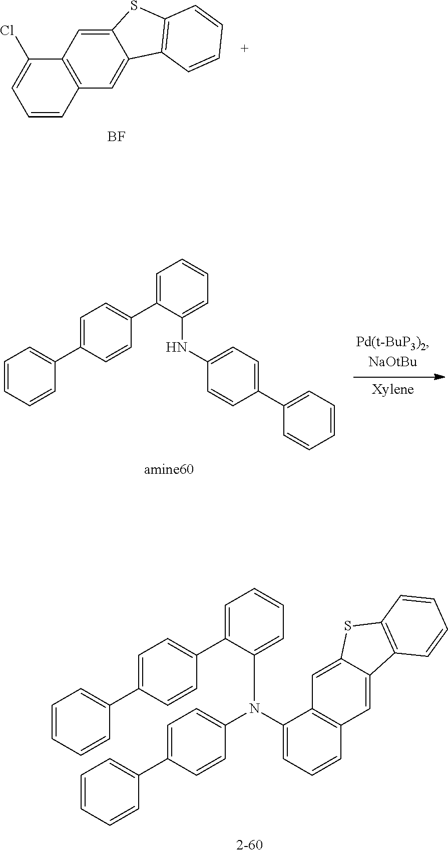

Compound BF (10 g, 37.2 mmol), amine 60 (14.8 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 15.2 g of Compound 2-60. (Yield: 65%, MS: [M+H]+=630)

### Synthesis Example 2-61

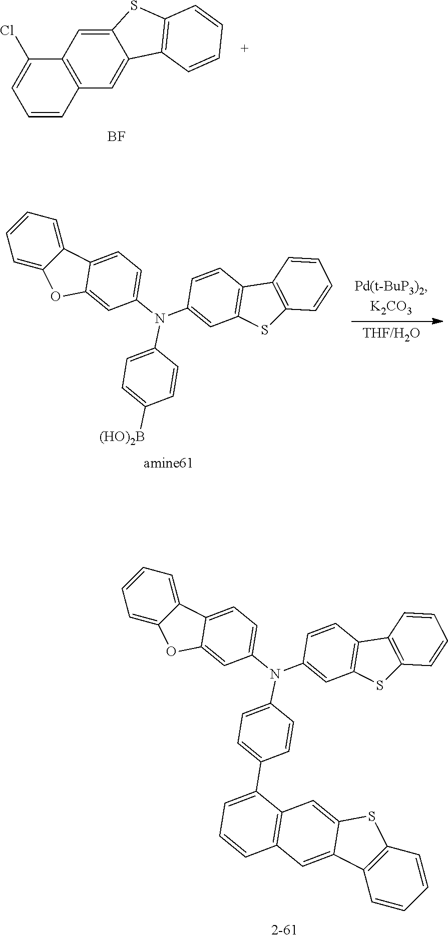

Compound BF (15 g, 55.8 mmol) and amine 61 (28.4 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.2 g of Compound 2-61. (Yield: 67%, MS: [M+H]+=674)

### Synthesis Example 2-62

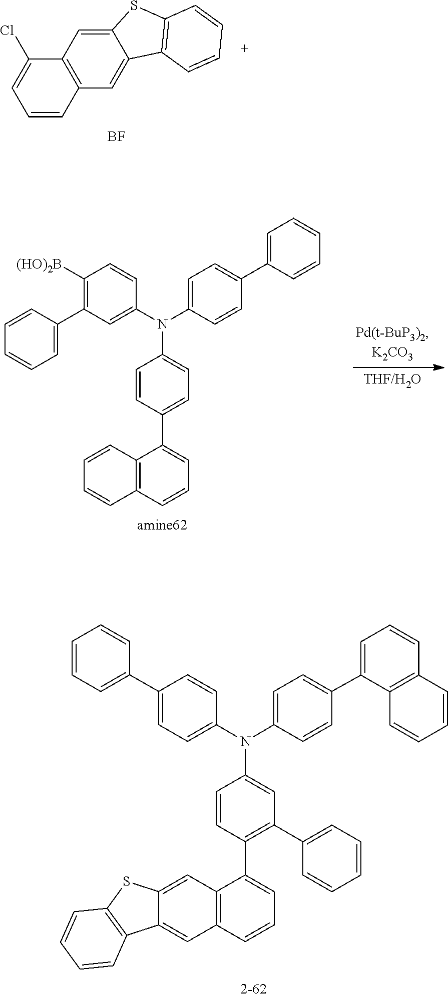

Compound BF (15 g, 55.8 mmol) and amine 62 (33.2 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 5 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 27.4 g of Compound 2-62. (Yield: 65%, MS: [M+H]+=756)

### Synthesis Example 2-63

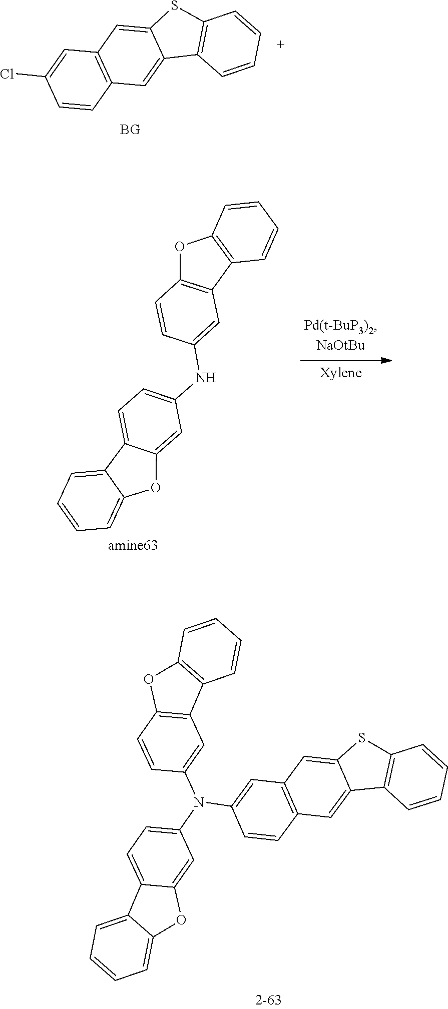

Compound BG (10 g, 37.2 mmol), amine 63 (13 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 13.2 g of Compound 2-63. (Yield: 61%, MS: [M+H]+=582)

### Synthesis Example 2-64

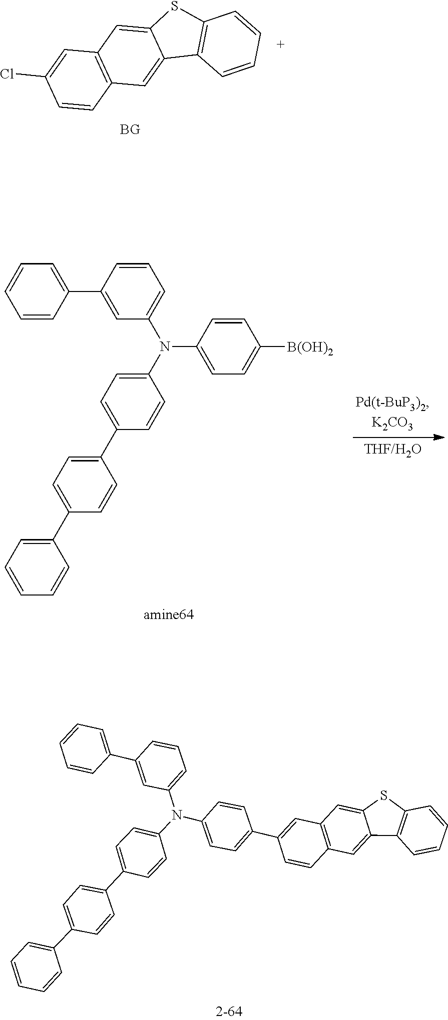

Compound BG (15 g, 55.8 mmol) and amine 64 (30.3 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 26 g of Compound 2-64. (Yield: 66%, MS: [M+H]+=706)

### Synthesis Example 2-65

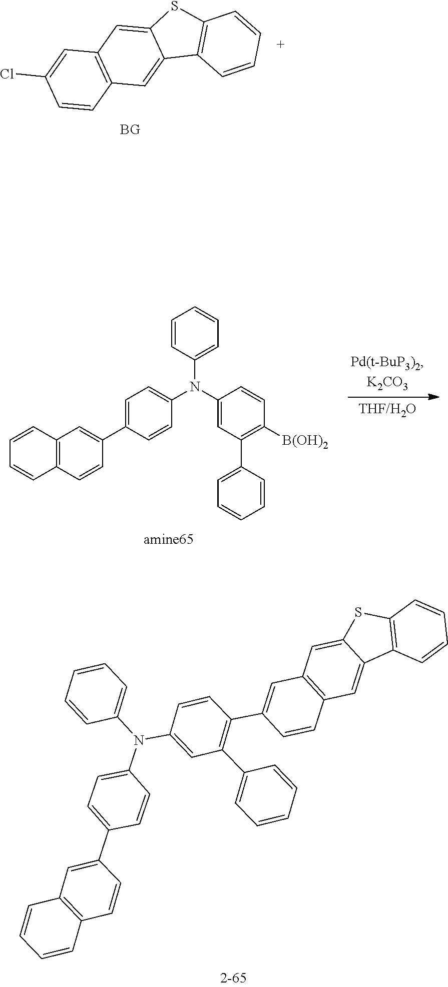

Compound BG (15 g, 55.8 mmol) and amine 65 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.4 g of Compound 2-65. (Yield: 67%, MS: [M+H]+=680)

### Synthesis Example 2-66

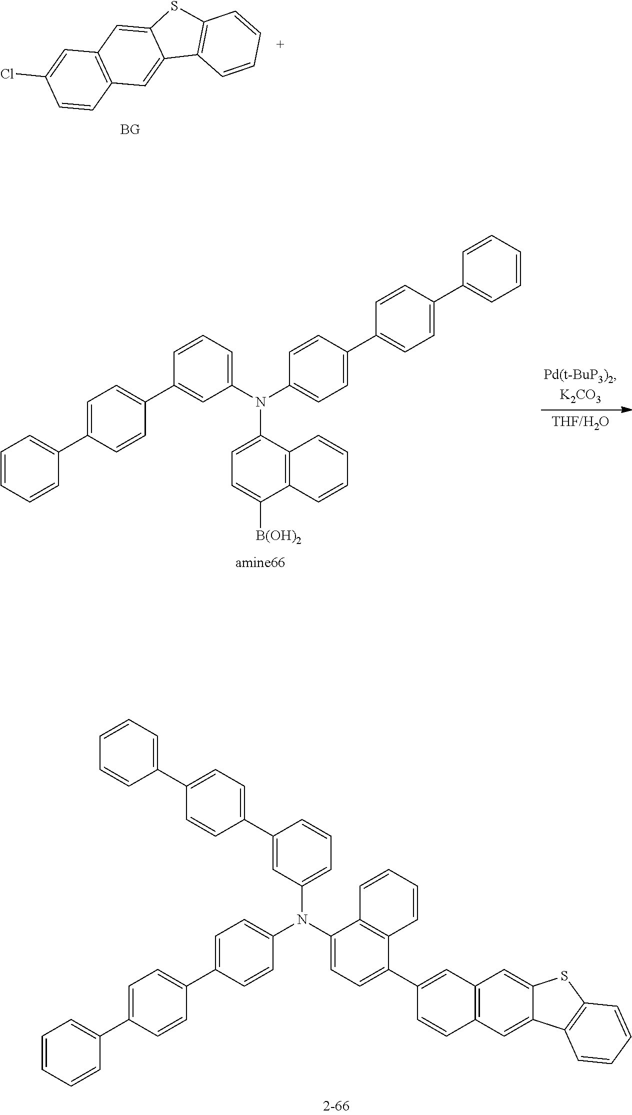

Compound BG (15 g, 55.8 mmol) and amine 66 (37.7 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 30.6 g of Compound 2-66. (Yield: 66%, MS: [M+H]+=832)

### Synthesis Example 2-67

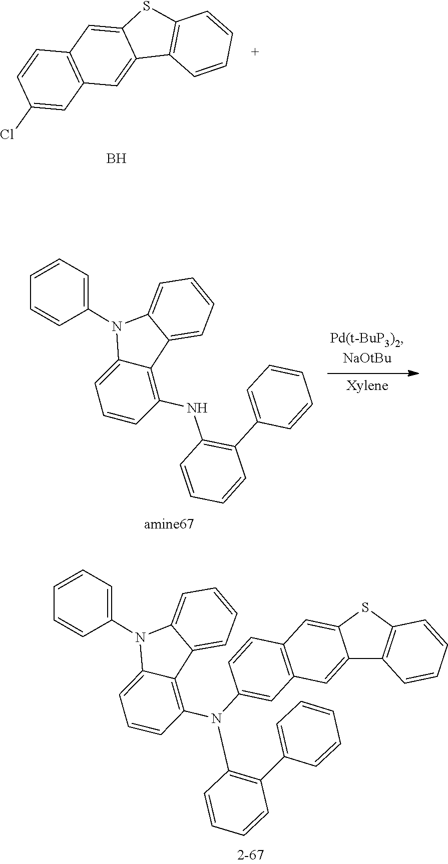

Compound BH (10 g, 37.2 mmol), amine 67 (15.3 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 3 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16.5 g of Compound 2-67. (Yield: 69%, MS: [M+H]+=643)

### Synthesis Example 2-68

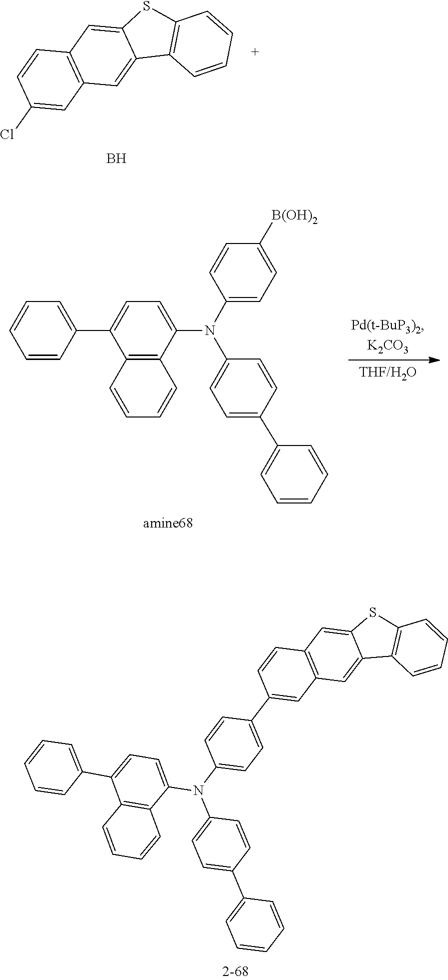

Compound BH (15 g, 55.8 mmol) and amine 68 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 28.4 g of Compound 2-68. (Yield: 75%, MS: [M+H]+=680)

### Synthesis Example 2-69

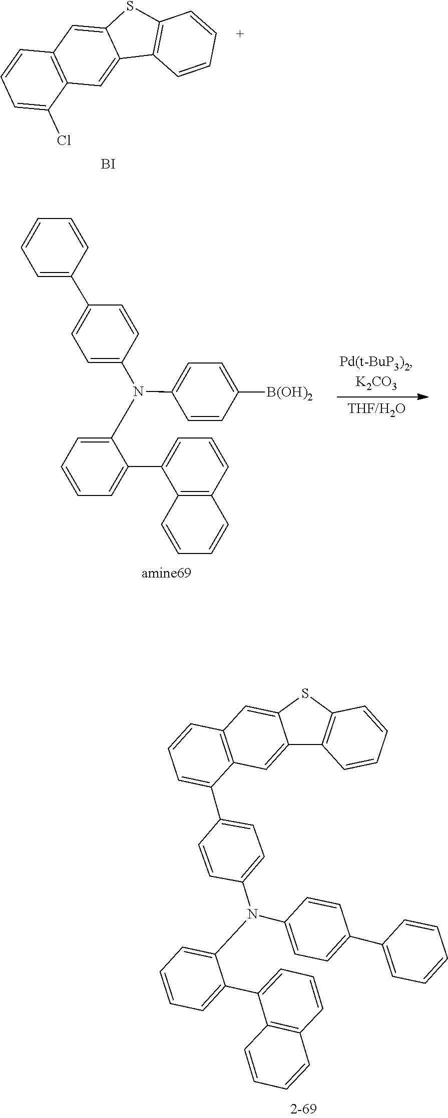

Compound BI (15 g, 55.8 mmol) and amine 69 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25 g of Compound 2-69. (Yield: 66%, MS: [M+H]+=680)

### Synthesis Example 2-70

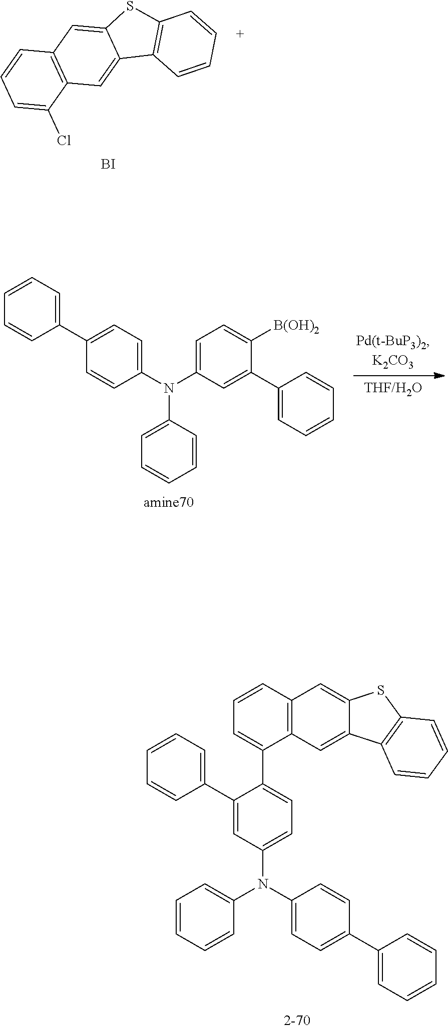

Compound BI (15 g, 55.8 mmol) and amine 70 (25.9 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 3 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 23.2 g of Compound 2-70. (Yield: 66%, MS: [M+H]+=630)

### Synthesis Example 2-71

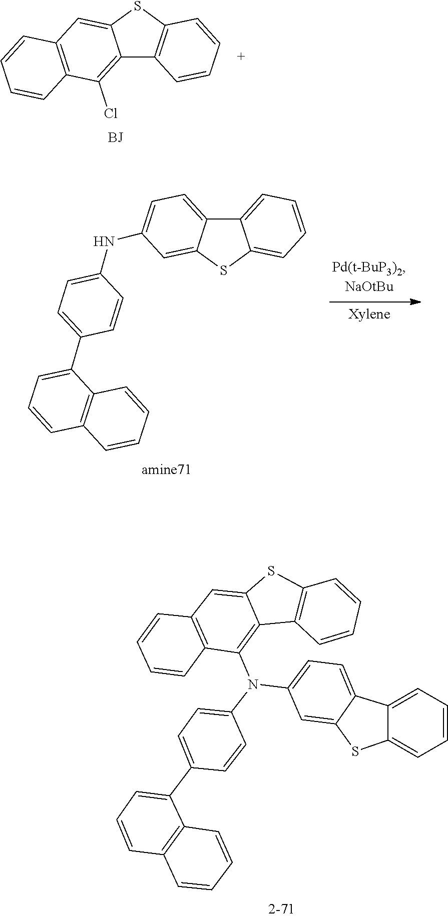

Compound BJ (10 g, 37.2 mmol), amine 71 (14.9 g, 37.2 mmol) and sodium tert-butoxide (11.8 g, 55.8 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. When the reaction was completed after 2 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate and then filtered, and the filtrate was distilled under reduced pressure. The concentrated compound was purified by silica gel column chromatography to prepare 16 g of Compound 2-71. (Yield: 68%, MS: [M+H]+=634)

### Synthesis Example 2-72

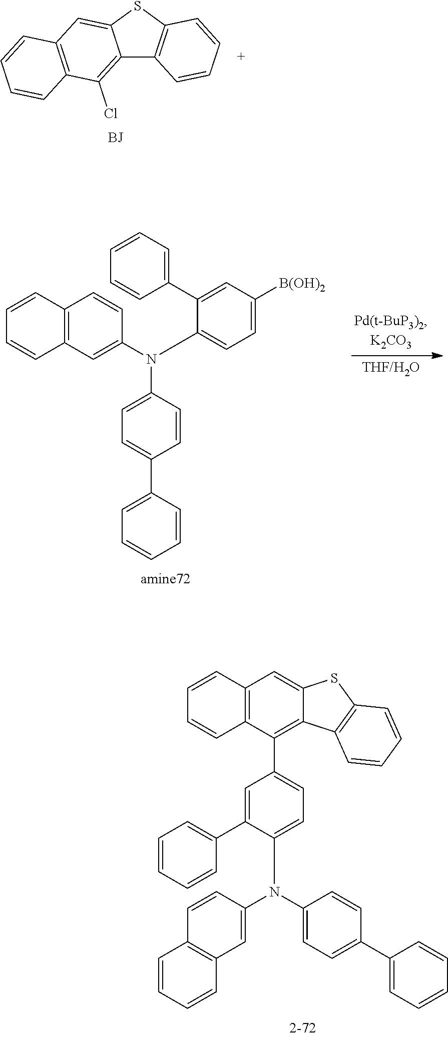

Compound BJ (15 g, 55.8 mmol) and amine 72 (28.8 g, 58.6 mmol) were added to 300 ml of THF, and the mixture was stirred and refluxed. Then, potassium carbonate (23.1 g, 167.4 mmol) was dissolved in 69 ml of water and added thereto, and the mixture was sufficiently stirred and then bis(tri-tert-butylphosphine)palladium(0) (0.3 g, 0.6 mmol) was added. After the reaction for 4 hours, the reaction mixture was cooled to room temperature, and the organic layer and the aqueous layer were separated and then the organic layer was distilled. This was again dissolved in chloroform, washed twice with water, and then the organic layer was separated, anhydrous magnesium sulfate was added thereto, stirred, and then filtered, and the filtrate was distilled under reduced pressure. The concentrated compound was purified by silica gel column chromatography to prepare 25.8 g of Compound 2-72. (Yield: 68%, MS: [M+H]+=680)

### Example 1

A glass substrate on which a thin film of ITO (indium tin oxide) was coated in a thickness of 1,000 Å was put into distilled water containing the detergent dissolved therein and washed by the ultrasonic wave. In this case, the used detergent was a product commercially available from Fischer Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following compound HI-1 was formed in a thickness of 1150 Å as a hole injection layer, but the following compound A-1 was p-doped at a concentration of 1.5 wt. %. The following compound HT-1 was vacuum deposited on the hole injection layer to form a hole transport layer with a film thickness of 800 Å. Then, the following compound EB-1 was vacuum deposited on the hole transport layer to a film thickness of 150 Å to form an electron blocking layer. Then, as a first host, a second host and a dopant, Compound 1-1 and Compound 2-1 prepared in the previous Synthesis Examples, and Compound Dp-7 were vacuum deposited in a weight ratio of 49:49:2 on the electron blocking layer to form a red light emitting layer with a film thickness of 400 Å. The following compound HB-1 was vacuum deposited on the light emitting layer to a film thickness of 30 Å to form a hole blocking layer. Then, the following compound ET-1 and the following compound LiQ were vacuum deposited in a weight ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

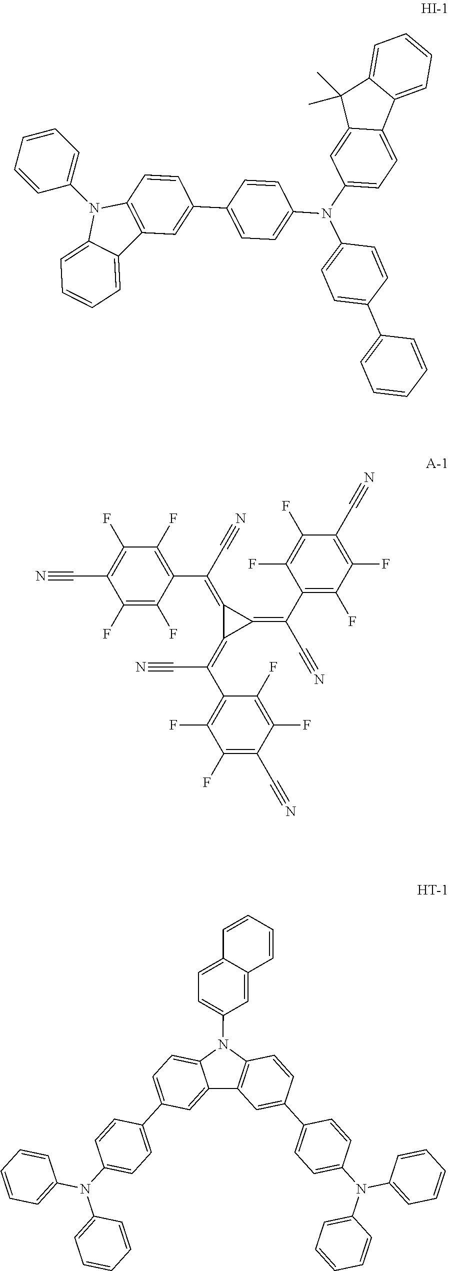 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(N(c2ccccc2)c2ccc(-c3ccc4c(c3)c3cc(-c5ccc(N(c6ccccc6)c6ccccc6)cc5)ccc3n4-c3ccc4ccccc4c3)cc2)cc1]

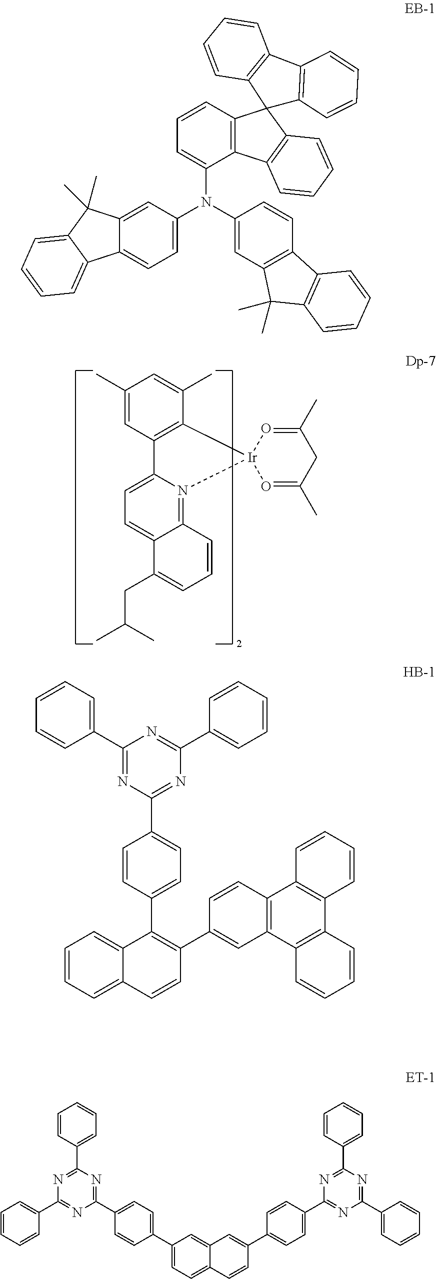

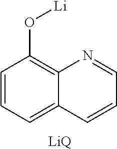

In the above-mentioned processes, the deposition rates of the organic materials were maintained at 0.4-0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷ to 5×10⁻⁶ torr, thereby manufacturing an organic light emitting device.

### Examples 2 to 390 and Comparative Examples 1 to 156

The organic light emitting devices of Examples 2 to 390 and Comparative Examples 1 to 156 were manufactured in the same manner as in Example 1, except that in the formation of the light emitting layer, the compounds shown in Tables 1 to 36 below were used as the first host and the second host.

The structures of the compounds used in Comparative Examples 1 to 156 are as follows.

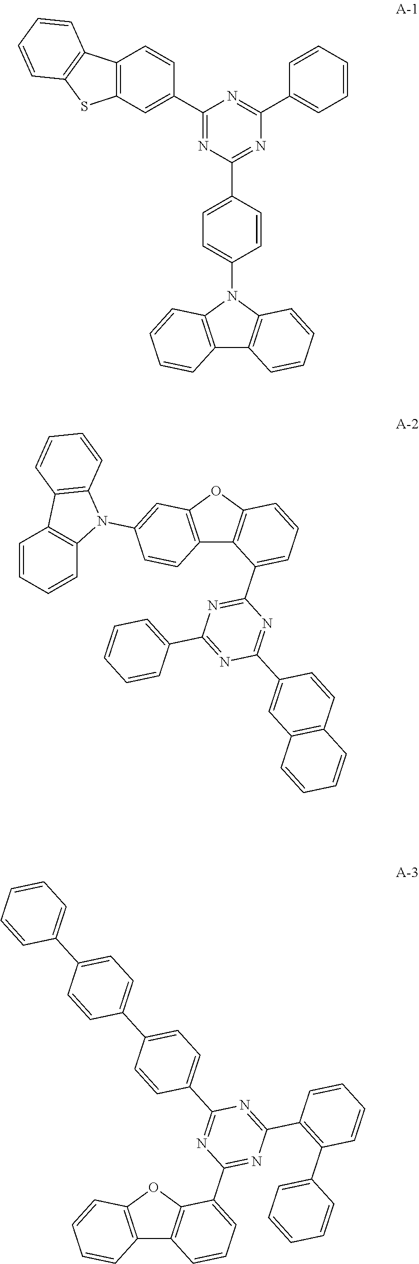 [SMILES: c1ccc(-c2nc(-c3ccc(-n4c5ccccc5c5ccccc54)cc3)nc(-c3ccc4c(c3)sc3ccccc34)n2)cc1 ; c1ccc(-c2nc(-c3ccc4ccccc4c3)nc(-c3cccc4oc5cc(-n6c7ccccc7c7ccccc76)ccc5c34)n2)cc1 ; c1ccc(-c2ccc(-c3ccc(-c4nc(-c5ccccc5-c5ccccc5)nc(-c5cccc6c5oc5ccccc56)n4)cc3)cc2)cc1]

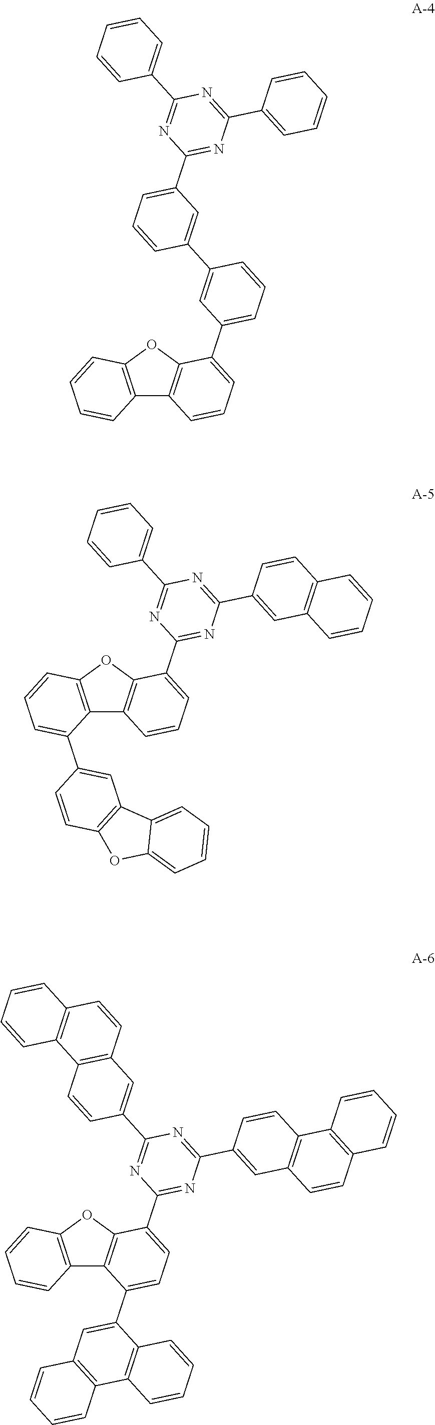 [SMILES: c1ccc(-c2nc(-c3ccccc3)nc(-c3cccc(-c4cccc(-c5cccc6c5oc5ccccc56)c4)c3)n2)cc1 ; c1ccc(-c2nc(-c3ccc4ccccc4c3)nc(-c3cccc4c3oc3cccc(-c5ccc6oc7ccccc7c6c5)c34)n2)cc1 ; c1ccc2c(c1)ccc1cc(-c3nc(-c4ccc5c(ccc6ccccc65)c4)nc(-c4ccc(-c5cc6ccccc6c6ccccc56)c5c4oc4ccccc45)n3)ccc12]

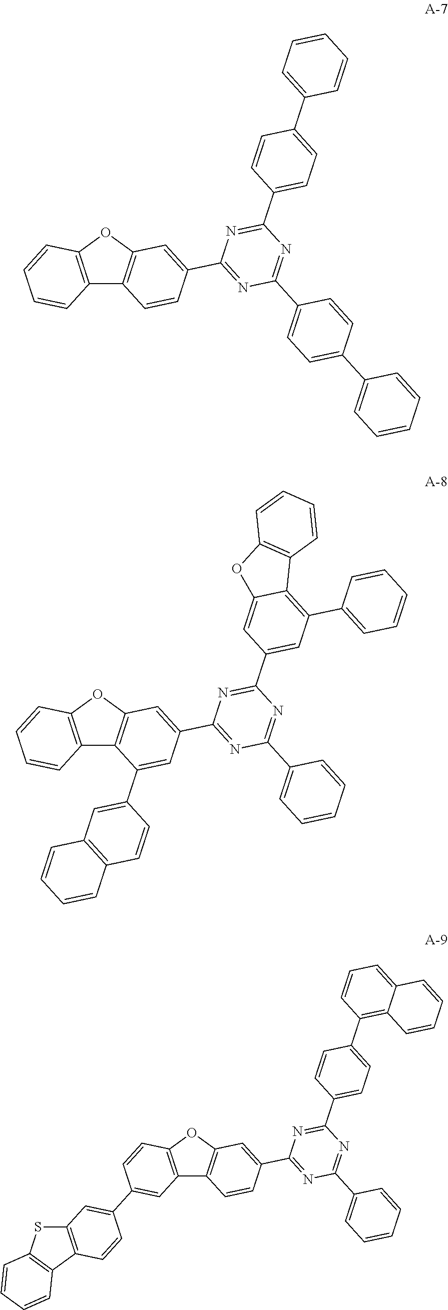 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2nc(-c3cc(-c4ccccc4)c4c(c3)oc3ccccc34)nc(-c3cc(-c4ccc5ccccc5c4)c4c(c3)oc3ccccc34)n2)cc1 ; c1ccc(-c2nc(-c3ccc(-c4cccc5ccccc45)cc3)nc(-c3ccc4c(c3)oc3ccc(-c5ccc6c(c5)sc5ccccc56)cc34)n2)cc1]

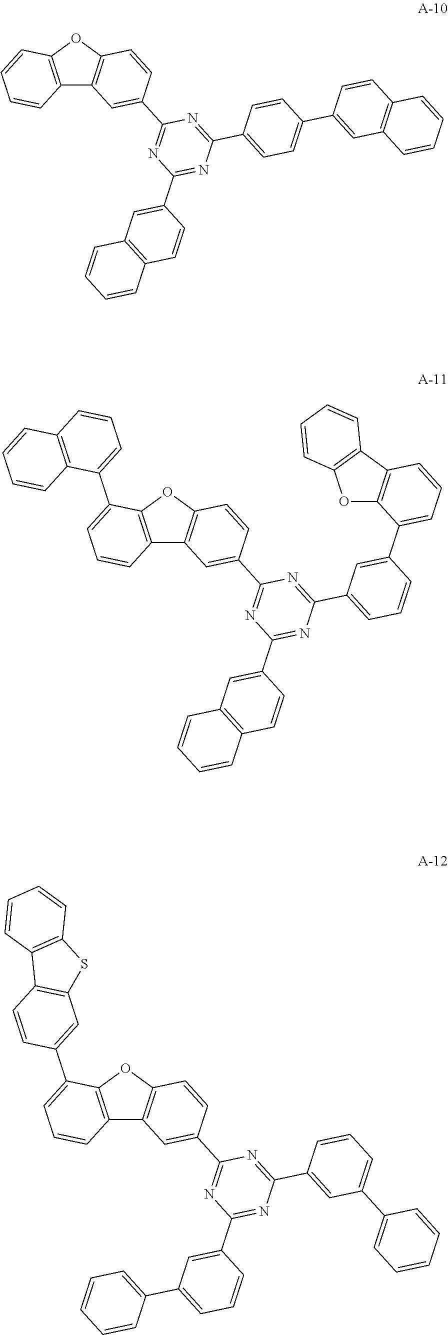 [SMILES: c1ccc2cc(-c3ccc(-c4nc(-c5ccc6ccccc6c5)nc(-c5ccc6oc7ccccc7c6c5)n4)cc3)ccc2c1 ; c1cc(-c2nc(-c3ccc4ccccc4c3)nc(-c3ccc4oc5c(-c6cccc7ccccc67)cccc5c4c3)n2)cc(-c2cccc3c2oc2ccccc23)c1 ; c1ccc(-c2cccc(-c3nc(-c4cccc(-c5ccccc5)c4)nc(-c4ccc5oc6c(-c7ccc8c(c7)sc7ccccc78)cccc6c5c4)n3)c2)cc1]

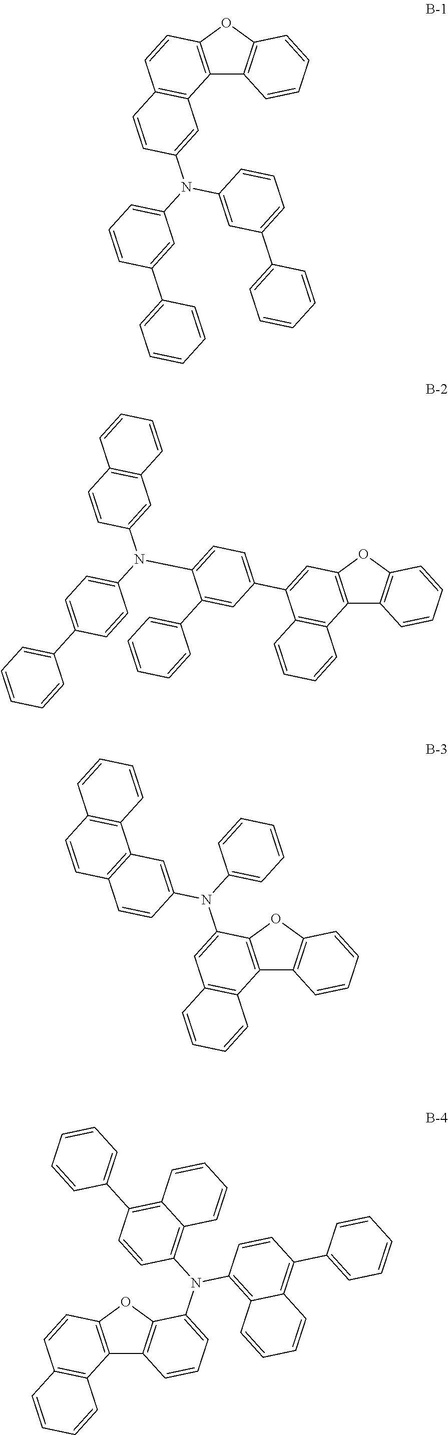 [SMILES: c1ccc(-c2cccc(N(c3cccc(-c4ccccc4)c3)c3ccc4ccc5oc6ccccc6c5c4c3)c2)cc1 ; c1ccc(-c2ccc(N(c3ccc4ccccc4c3)c3ccc(-c4cc5oc6ccccc6c5c5ccccc45)cc3-c3ccccc3)cc2)cc1 ; c1ccc(N(c2ccc3ccc4ccccc4c3c2)c2cc3ccccc3c3c2oc2ccccc23)cc1 ; c1ccc(-c2ccc(N(c3ccc(-c4ccccc4)c4ccccc34)c3cccc4c3oc3ccc5ccccc5c34)c3ccccc23)cc1]

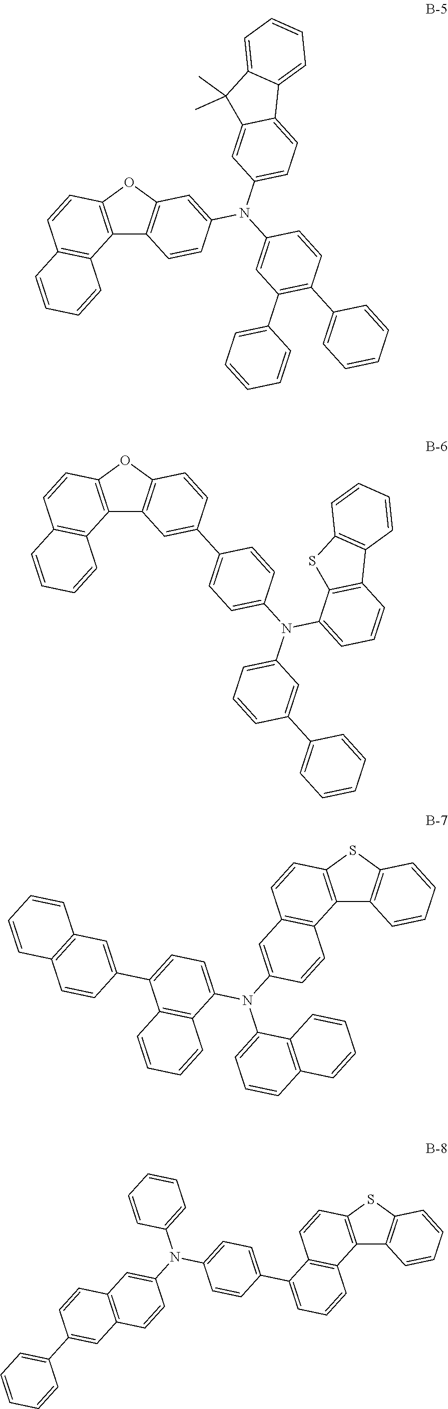 [SMILES: CC1(C)c2ccccc2-c2ccc(N(c3ccc(-c4ccccc4)c(-c4ccccc4)c3)c3ccc4c(c3)oc3ccc5ccccc5c34)cc21 ; c1ccc(-c2cccc(N(c3ccc(-c4ccc5oc6ccc7ccccc7c6c5c4)cc3)c3cccc4c3sc3ccccc34)c2)cc1 ; c1ccc2cc(-c3ccc(N(c4ccc5c(ccc6sc7ccccc7c65)c4)c4cccc5ccccc45)c4ccccc34)ccc2c1 ; c1ccc(-c2ccc3cc(N(c4ccccc4)c4ccc(-c5cccc6c5ccc5sc7ccccc7c56)cc4)ccc3c2)cc1]

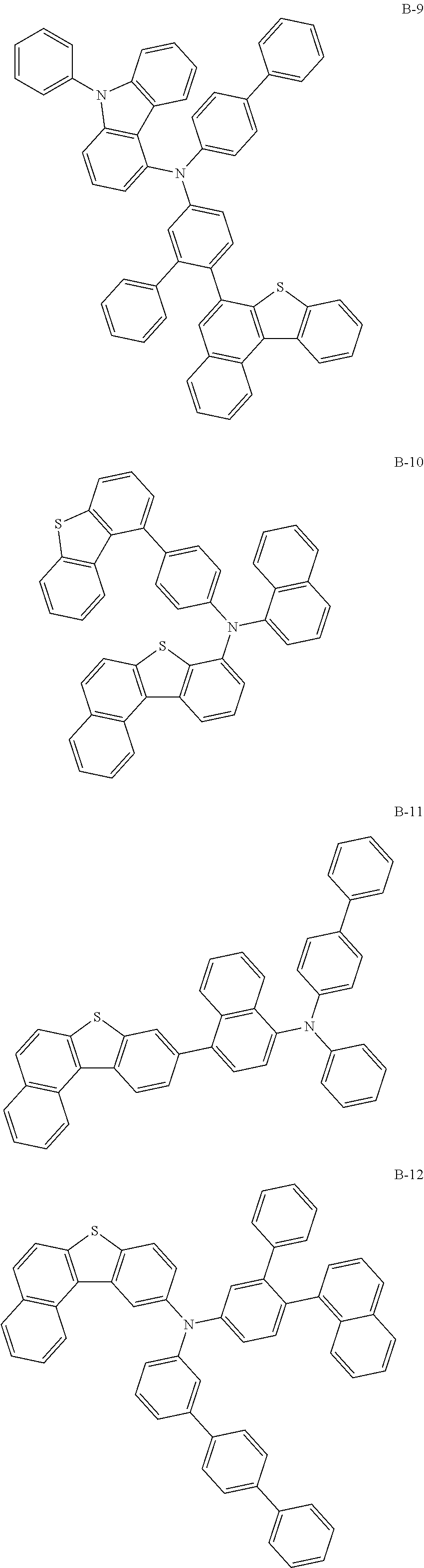 [SMILES: c1ccc(-c2ccc(N(c3ccc(-c4cc5ccccc5c5c4sc4ccccc45)c(-c4ccccc4)c3)c3cccc4c3c3ccccc3n4-c3ccccc3)cc2)cc1 ; c1ccc2c(N(c3ccc(-c4cccc5sc6ccccc6c45)cc3)c3cccc4c3sc3ccc5ccccc5c34)cccc2c1 ; c1ccc(-c2ccc(N(c3ccccc3)c3ccc(-c4ccc5c(c4)sc4ccc6ccccc6c45)c4ccccc34)cc2)cc1 ; c1ccc(-c2ccc(-c3cccc(N(c4ccc(-c5cccc6ccccc56)c(-c5ccccc5)c4)c4ccc5sc6ccc7ccccc7c6c5c4)c3)cc2)cc1]

### Test Example: Evaluation of Device Characteristics

The voltage, efficiency and lifetime (T95) were measured by applying a current to the organic light emitting devices manufactured in Examples 1 to 390 and Comparative Examples 1 to 156, and the results are shown in Tables 1 to 36 below. At this time, the measurement was performed by a current density of 15 mA/cm², and the lifetime (T95) means the time (hr) required for the luminance to be reduced to 95% of the initial luminance based on 7000 nit.

TABLE 1

| | | | Driving | | | |
|---|---|---|---|---|---|---|
| | | | voltage | Efficiency | Lifetime | Luminescent |
| Category | First host | Second host | (V) | (cd/A) | T95 (hr) | color |
| Example 1 | Compound 1-1 | Compound 2-1 | 3.44 | 23.98 | 298 | Red |
| Example 2 | | Compound 2-20 | 3.46 | 24.00 | 283 | Red |
| Example 3 | | Compound 2-29 | 3.40 | 24.16 | 298 | Red |
| Example 4 | | Compound 2-37 | 3.45 | 23.91 | 294 | Red |
| Example 5 | | Compound 2-51 | 3.41 | 24.06 | 308 | Red |
| Example 6 | Compound 1-2 | Compound 2-2 | 3.63 | 22.00 | 252 | Red |
| Example 7 | | Compound 2-23 | 3.60 | 21.87 | 251 | Red |
| Example 8 | | Compound 2-30 | 3.62 | 21.73 | 241 | Red |
| Example 9 | | Compound 2-38 | 3.62 | 21.18 | 245 | Red |
| Example 10 | | Compound 2-52 | 3.63 | 21.38 | 222 | Red |
| Example 11 | Compound 1-3 | Compound 2-3 | 3.60 | 21.21 | 243 | Red |
| Example 12 | | Compound 2-16 | 3.61 | 21.93 | 221 | Red |
| Example 13 | | Compound 2-31 | 3.61 | 21.44 | 249 | Red |
| Example 14 | | Compound 2-39 | 3.62 | 21.91 | 252 | Red |
| Example 15 | | Compound 2-53 | 3.59 | 21.72 | 245 | Red |

TABLE 2

| | | | Driving | | | |
|---|---|---|---|---|---|---|
| | | | voltage | Efficiency | Lifetime | Luminescent |
| Category | First host | Second host | (V) | (cd/A) | T95 (hr) | color |
| Example 16 | Compound 1-4 | Compound 2-4 | 3.59 | 22.86 | 290 | Red |
| Example 17 | | Compound 2-21 | 3.54 | 22.98 | 278 | Red |
| Example 18 | | Compound 2-32 | 3.57 | 23.92 | 298 | Red |
| Example 19 | | Compound 2-40 | 3.53 | 23.11 | 275 | Red |
| Example 20 | | Compound 2-54 | 3.59 | 23.97 | 302 | Red |
| Example 21 | Compound 1-5 | Compound 2-5 | 3.61 | 23.96 | 293 | Red |
| Example 22 | | Compound 2-19 | 3.60 | 24.05 | 286 | Red |
| Example 23 | | Compound 2-33 | 3.59 | 22.94 | 281 | Red |
| Example 24 | | Compound 2-41 | 3.54 | 24.07 | 300 | Red |
| Example 25 | | Compound 2-55 | 3.53 | 23.11 | 278 | Red |
| Example 26 | Compound 1-6 | Compound 2-6 | 3.44 | 22.86 | 298 | Red |
| Example 27 | | Compound 2-17 | 3.46 | 22.98 | 310 | Red |
| Example 28 | | Compound 2-34 | 3.40 | 23.92 | 298 | Red |
| Example 29 | | Compound 2-42 | 3.45 | 23.11 | 314 | Red |
| Example 30 | | Compound 2-56 | 3.41 | 23.97 | 328 | Red |

TABLE 3

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.47 | 24.11 | 304 | Red |
| 31 | 1-7 | 2-7 | | | | |
| Example | | Compound | 3.43 | 24.15 | 298 | Red |
| 32 | | 2-35 | | | | |
| Example | | Compound | 3.42 | 24.17 | 306 | Red |
| 33 | | 2-2 | | | | |
| Example | | Compound | 3.39 | 23.50 | 337 | Red |
| 34 | | 2-49 | | | | |
| Example | | Compound | 3.47 | 23.69 | 309 | Red |
| 35 | | 2-57 | | | | |
| Example | Compound | Compound | 3.52 | 22.13 | 271 | Red |
| 36 | 1-8 | 2-8 | | | | |
| Example | | Compound | 3.45 | 23.02 | 238 | Red |
| 37 | | 2-36 | | | | |
| Example | | Compound | 3.51 | 22.82 | 261 | Red |
| 38 | | 2-65 | | | | |
| Example | | Compound | 3.45 | 23.02 | 277 | Red |
| 39 | | 2-44 | | | | |
| Example | | Compound | 3.54 | 22.00 | 275 | Red |
| 40 | | 2-58 | | | | |
| Example | Compound | Compound | 3.47 | 22.58 | 239 | Red |
| 41 | 1-9 | 2-9 | | | | |
| Example | | Compound | 3.52 | 22.52 | 236 | Red |
| 42 | | 2-18 | | | | |
| Example | | Compound | 3.52 | 22.21 | 248 | Red |
| 43 | | 2-22 | | | | |
| Example | | Compound | 3.51 | 23.07 | 269 | Red |
| 44 | | 2-45 | | | | |
| Example | | Compound | 3.49 | 22.83 | 242 | Red |
| 45 | | 2-66 | | | | |

TABLE 4

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.39 | 23.37 | 323 | Red |
| 46 | 1-10 | 2-10 | | | | |
| Example | | Compound | 3.42 | 23.03 | 337 | Red |
| 47 | | 2-24 | | | | |
| Example | | Compound | 3.39 | 22.81 | 325 | Red |
| 48 | | 2-72 | | | | |
| Example | | Compound | 3.40 | 23.21 | 324 | Red |
| 49 | | 2-59 | | | | |
| Example | | Compound | 3.37 | 24.09 | 299 | Red |
| 50 | | 2-65 | | | | |
| Example | Compound | Compound | 3.43 | 22.93 | 303 | Red |
| 51 | 1-11 | 2-11 | | | | |
| Example | | Compound | 3.47 | 22.83 | 300 | Red |
| 52 | | 2-25 | | | | |
| Example | | Compound | 3.46 | 23.77 | 330 | Red |
| 53 | | 2-67 | | | | |
| Example | | Compound | 3.45 | 23.84 | 301 | Red |
| 54 | | 2-46 | | | | |
| Example | | Compound | 3.38 | 22.99 | 302 | Red |
| 55 | | 2-60 | | | | |
| Example | Compound | Compound | 3.48 | 24.30 | 304 | Red |
| 56 | 1-12 | 2-12 | | | | |
| Example | | Compound | 3.42 | 23.89 | 302 | Red |
| 57 | | 2-26 | | | | |
| Example | | Compound | 3.41 | 24.32 | 311 | Red |
| 58 | | 2-31 | | | | |
| Example | | Compound | 3.38 | 24.46 | 292 | Red |
| 59 | | 2-47 | | | | |
| Example | | Compound | 3.39 | 23.58 | 294 | Red |
| 60 | | 2-61 | | | | |

TABLE 5

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.52 | 22.86 | 250 | Red |
| 61 | 1-13 | 2-13 | | | | |
| Example | | Compound | 3.53 | 22.06 | 263 | Red |
| 62 | | 2-27 | | | | |
| Example | | Compound | 3.50 | 22.02 | 248 | Red |
| 63 | | 2-68 | | | | |
| Example | | Compound | 3.54 | 22.70 | 274 | Red |
| 64 | | 2-48 | | | | |
| Example | | Compound | 3.45 | 22.34 | 285 | Red |
| 65 | | 2-62 | | | | |
| Example | Compound | Compound | 3.49 | 22.70 | 249 | Red |
| 66 | 1-14 | 2-14 | | | | |
| Example | | Compound | 3.53 | 22.56 | 280 | Red |
| 67 | | 2-28 | | | | |
| Example | | Compound | 3.51 | 22.56 | 274 | Red |
| 68 | | 2-69 | | | | |
| Example | | Compound | 3.48 | 22.03 | 235 | Red |
| 69 | | 2-49 | | | | |
| Example | | Compound | 3.52 | 22.88 | 254 | Red |
| 70 | | 2-63 | | | | |
| Example | Compound | Compound | 3.53 | 24.03 | 291 | Red |
| 71 | 1-15 | 2-15 | | | | |
| Example | | Compound | 3.54 | 23.99 | 275 | Red |
| 72 | | 2-29 | | | | |
| Example | | Compound | 3.56 | 23.19 | 297 | Red |
| 73 | | 2-70 | | | | |
| Example | | Compound | 3.60 | 22.90 | 295 | Red |
| 74 | | 2-50 | | | | |
| Example | | Compound | 3.56 | 23.44 | 288 | Red |
| 75 | | 2-64 | | | | |

TABLE 6

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.60 | 22.91 | 233 | Red |
| 76 | 1-16 | 2-6 | | | | |
| Example | | Compound | 3.53 | 22.74 | 238 | Red |
| 77 | | 2-17 | | | | |
| Example | | Compound | 3.61 | 22.41 | 226 | Red |
| 78 | | 2-34 | | | | |
| Example | | Compound | 3.53 | 22.18 | 227 | Red |
| 79 | | 2-42 | | | | |
| Example | | Compound | 3.58 | 22.37 | 237 | Red |
| 80 | | 2-56 | | | | |
| Example | Compound | Compound | 3.58 | 22.85 | 246 | Red |
| 81 | 1-17 | 2-7 | | | | |
| Example | | Compound | 3.53 | 23.03 | 232 | Red |
| 82 | | 2-35 | | | | |
| Example | | Compound | 3.58 | 22.40 | 228 | Red |
| 83 | | 2-2 | | | | |
| Example | | Compound | 3.53 | 22.50 | 234 | Red |
| 84 | | 2-43 | | | | |
| Example | | Compound | 3.54 | 22.61 | 229 | Red |
| 85 | | 2-57 | | | | |
| Example | Compound | Compound | 3.46 | 24.29 | 308 | Red |
| 86 | 1-18 | 2-8 | | | | |
| Example | | Compound | 3.44 | 23.84 | 319 | Red |
| 87 | | 2-36 | | | | |
| Example | | Compound | 3.48 | 23.89 | 332 | Red |
| 88 | | 2-65 | | | | |
| Example | | Compound | 3.48 | 23.65 | 295 | Red |
| 89 | | 2-44 | | | | |
| Example | | Compound | 3.43 | 24.04 | 343 | Red |
| 90 | | 2-51 | | | | |

TABLE 7

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.37 | 23.68 | 297 | Red |
| 91 | 1-19 | 2-9 | | | | |
| Example | | Compound | 3.44 | 24.01 | 312 | Red |
| 92 | | 2-18 | | | | |
| Example | | Compound | 3.39 | 22.95 | 325 | Red |
| 93 | | 2-22 | | | | |
| Example | | Compound | 3.47 | 23.18 | 309 | Red |
| 94 | | 2-45 | | | | |
| Example | | Compound | 3.41 | 23.37 | 340 | Red |
| 95 | | 2-66 | | | | |
| Example | Compound | Compound | 3.46 | 23.50 | 336 | Red |
| 96 | 1-20 | 2-10 | | | | |
| Example | | Compound | 3.47 | 22.80 | 321 | Red |
| 97 | | 2-24 | | | | |
| Example | | Compound | 3.41 | 23.62 | 334 | Red |
| 98 | | 2-72 | | | | |
| Example | | Compound | 3.39 | 23.84 | 295 | Red |
| 99 | | 2-59 | | | | |
| Example | | Compound | 3.39 | 24.04 | 307 | Red |
| 100 | | 2-65 | | | | |
| Example | Compound | Compound | 3.54 | 22.88 | 296 | Red |
| 101 | 1-21 | 2-1 | | | | |
| Example | | Compound | 3.52 | 22.80 | 298 | Red |
| 102 | | 2-20 | | | | |
| Example | | Compound | 3.52 | 24.02 | 303 | Red |
| 103 | | 2-29 | | | | |
| Example | | Compound | 3.59 | 23.97 | 294 | Red |
| 104 | | 2-37 | | | | |
| Example | | Compound | 3.53 | 22.91 | 302 | Red |
| 105 | | 2-51 | | | | |

TABLE 8

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.61 | 23.48 | 302 | Red |
| 106 | 1-22 | 2-2 | | | | |
| Example | | Compound | 3.56 | 23.16 | 294 | Red |
| 107 | | 2-23 | | | | |
| Example | | Compound | 3.54 | 24.00 | 305 | Red |
| 108 | | 2-30 | | | | |
| Example | | Compound | 3.58 | 23.10 | 285 | Red |
| 109 | | 2-38 | | | | |
| Example | | Compound | 3.56 | 23.36 | 296 | Red |
| 110 | | 2-52 | | | | |
| Example | Compound | Compound | 3.46 | 24.51 | 304 | Red |
| 111 | 1-23 | 2-3 | | | | |
| Example | | Compound | 3.44 | 24.05 | 334 | Red |
| 112 | | 2-16 | | | | |
| Example | | Compound | 3.42 | 24.34 | 361 | Red |
| 113 | | 2-31 | | | | |
| Example | | Compound | 3.45 | 24.06 | 321 | Red |
| 114 | | 2-39 | | | | |
| Example | | Compound | 3.43 | 24.39 | 303 | Red |
| 115 | | 2-53 | | | | |
| Example | Compound | Compound | 3.60 | 23.45 | 302 | Red |
| 116 | 1-24 | 2-4 | | | | |
| Example | | Compound | 3.55 | 23.92 | 282 | Red |
| 117 | | 2-21 | | | | |
| Example | | Compound | 3.57 | 23.67 | 291 | Red |
| 118 | | 2-32 | | | | |
| Example | | Compound | 3.57 | 24.05 | 281 | Red |
| 119 | | 2-40 | | | | |
| Example | | Compound | 3.57 | 23.90 | 295 | Red |
| 120 | | 2-54 | | | | |

TABLE 9

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.60 | 22.66 | 251 | Red |
| 121 | 1-25 | 2-5 | | | | |
| Example | | Compound | 3.55 | 22.48 | 255 | Red |
| 122 | | 2-19 | | | | |
| Example | | Compound | 3.57 | 22.28 | 243 | Red |
| 123 | | 2-33 | | | | |
| Example | | Compound | 3.57 | 23.08 | 236 | Red |
| 124 | | 2-41 | | | | |
| Example | | Compound | 3.57 | 22.65 | 229 | Red |
| 125 | | 2-55 | | | | |
| Example | Compound | Compound | 3.62 | 21.13 | 251 | Red |
| 126 | 1-26 | 2-6 | | | | |
| Example | | Compound | 3.59 | 21.93 | 255 | Red |
| 127 | | 2-17 | | | | |
| Example | | Compound | 3.59 | 21.32 | 243 | Red |
| 128 | | 2-34 | | | | |
| Example | | Compound | 3.60 | 21.67 | 236 | Red |
| 129 | | 2-42 | | | | |
| Example | | Compound | 3.59 | 21.65 | 229 | Red |
| 130 | | 2-56 | | | | |
| Example | Compound | Compound | 3.46 | 24.13 | 316 | Red |
| 131 | 1-27 | 2-2 | | | | |
| Example | | Compound | 3.37 | 24.10 | 323 | Red |
| 132 | | 2-35 | | | | |
| Example | | Compound | 3.42 | 23.55 | 336 | Red |
| 133 | | 2-7 | | | | |
| Example | | Compound | 3.45 | 23.87 | 337 | Red |
| 134 | | 2-43 | | | | |
| Example | | Compound | 3.44 | 24.48 | 315 | Red |
| 135 | | 2-57 | | | | |

TABLE 10

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.38 | 22.91 | 302 | Red |
| 136 | 1-28 | 2-8 | | | | |
| Example | | Compound | 3.46 | 22.86 | 337 | Red |
| 137 | | 2-36 | | | | |
| Example | | Compound | 3.38 | 23.48 | 321 | Red |
| 138 | | 2-65 | | | | |
| Example | | Compound | 3.47 | 23.32 | 338 | Red |
| 139 | | 2-44 | | | | |
| Example | | Compound | 3.37 | 22.94 | 323 | Red |
| 140 | | 2-58 | | | | |
| Example | Compound | Compound | 3.43 | 23.62 | 335 | Red |
| 141 | 1-29 | 2-9 | | | | |
| Example | | Compound | 3.48 | 23.96 | 332 | Red |
| 142 | | 2-18 | | | | |
| Example | | Compound | 3.47 | 22.97 | 339 | Red |
| 143 | | 2-22 | | | | |
| Example | | Compound | 3.48 | 23.80 | 322 | Red |
| 144 | | 2-45 | | | | |
| Example | | Compound | 3.44 | 23.56 | 330 | Red |
| 145 | | 2-66 | | | | |
| Example | Compound | Compound | 3.61 | 22.91 | 302 | Red |
| 146 | 1-30 | 2-10 | | | | |
| Example | | Compound | 3.56 | 22.86 | 291 | Red |
| 147 | | 2-24 | | | | |
| Example | | Compound | 3.61 | 23.48 | 276 | Red |
| 148 | | 2-72 | | | | |
| Example | | Compound | 3.59 | 23.32 | 283 | Red |
| 149 | | 2-59 | | | | |
| Example | | Compound | 3.56 | 22.94 | 295 | Red |
| 150 | | 2-65 | | | | |

TABLE 11

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.48 | 24.42 | 326 | Red |
| 151 | 1-31 | 2-11 | | | | |
| Example | | Compound | 3.40 | 24.31 | 326 | Red |
| 152 | | 2-25 | | | | |
| Example | | Compound | 3.40 | 23.59 | 338 | Red |
| 153 | | 2-67 | | | | |
| Example | | Compound | 3.43 | 24.46 | 318 | Red |
| 154 | | 2-46 | | | | |
| Example | | Compound | 3.48 | 23.82 | 336 | Red |
| 155 | | 2-60 | | | | |
| Example | Compound | Compound | 3.61 | 21.69 | 234 | Red |
| 156 | 1-32 | 2-12 | | | | |
| Example | | Compound | 3.62 | 21.47 | 237 | Red |
| 157 | | 2-26 | | | | |
| Example | | Compound | 3.59 | 21.51 | 244 | Red |
| 158 | | 2-71 | | | | |
| Example | | Compound | 3.59 | 22.09 | 246 | Red |
| 159 | | 2-47 | | | | |
| Example | | Compound | 3.60 | 22.00 | 243 | Red |
| 160 | | 2-61 | | | | |
| Example | Compound | Compound | 3.45 | 24.15 | 363 | Red |
| 161 | 1-33 | 2-2 | | | | |
| Example | | Compound | 3.44 | 23.76 | 334 | Red |
| 162 | | 2-13 | | | | |
| Example | | Compound | 3.42 | 23.96 | 331 | Red |
| 163 | | 2-27 | | | | |
| Example | | Compound | 3.42 | 24.21 | 307 | Red |
| 164 | | 2-48 | | | | |
| Example | | Compound | 3.47 | 24.34 | 296 | Red |
| 165 | | 2-62 | | | | |

TABLE 12

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.42 | 24.29 | 335 | Red |
| 166 | 1-34 | 2-14 | | | | |
| Example | | Compound | 3.45 | 24.07 | 306 | Red |
| 167 | | 2-28 | | | | |
| Example | | Compound | 3.47 | 23.57 | 333 | Red |
| 168 | | 2-69 | | | | |
| Example | | Compound | 3.37 | 23.53 | 363 | Red |
| 169 | | 2-49 | | | | |
| Example | | Compound | 3.39 | 23.61 | 305 | Red |
| 170 | | 2-63 | | | | |
| Example | Compound | Compound | 3.47 | 23.74 | 313 | Red |
| 171 | 1-35 | 2-15 | | | | |
| Example | | Compound | 3.39 | 24.16 | 314 | Red |
| 172 | | 2-29 | | | | |
| Example | | Compound | 3.44 | 24.43 | 316 | Red |
| 173 | | 2-70 | | | | |
| Example | | Compound | 3.41 | 23.67 | 340 | Red |
| 174 | | 2-50 | | | | |
| Example | | Compound | 3.46 | 23.53 | 305 | Red |
| 175 | | 2-64 | | | | |
| Example | Compound | Compound | 3.59 | 21.33 | 238 | Red |
| 176 | 1-37 | 2-2 | | | | |
| Example | | Compound | 3.63 | 21.53 | 238 | Red |
| 177 | | 2-23 | | | | |
| Example | | Compound | 3.60 | 21.39 | 233 | Red |
| 178 | | 2-30 | | | | |
| Example | | Compound | 3.62 | 22.00 | 232 | Red |
| 179 | | 2-38 | | | | |
| Example | | Compound | 3.61 | 21.36 | 227 | Red |
| 180 | | 2-52 | | | | |

TABLE 13

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.39 | 24.19 | 300 | Red |
| 181 | 1-38 | 2-2 | | | | |
| Example | | Compound | 3.46 | 24.46 | 318 | Red |
| 182 | | 2-16 | | | | |
| Example | | Compound | 3.41 | 24.47 | 312 | Red |
| 183 | | 2-30 | | | | |
| Example | | Compound | 3.40 | 23.71 | 297 | Red |
| 184 | | 2-38 | | | | |
| Example | | Compound | 3.41 | 24.07 | 306 | Red |
| 185 | | 2-52 | | | | |
| Example | Compound | Compound | 3.59 | 21.76 | 222 | Red |
| 186 | 1-39 | 2-4 | | | | |
| Example | | Compound | 3.60 | 21.50 | 249 | Red |
| 187 | | 2-21 | | | | |
| Example | | Compound | 3.63 | 21.84 | 248 | Red |
| 188 | | 2-32 | | | | |
| Example | | Compound | 3.63 | 21.46 | 223 | Red |
| 189 | | 2-40 | | | | |
| Example | | Compound | 3.60 | 21.36 | 245 | Red |
| 190 | | 2-54 | | | | |
| Example | Compound | Compound | 3.61 | 21.61 | 224 | Red |
| 191 | 1-40 | 2-5 | | | | |
| Example | | Compound | 3.59 | 22.08 | 236 | Red |
| 192 | | 2-19 | | | | |
| Example | | Compound | 3.60 | 21.78 | 227 | Red |
| 193 | | 2-33 | | | | |
| Example | | Compound | 3.60 | 21.65 | 227 | Red |
| 194 | | 2-41 | | | | |
| Example | | Compound | 3.63 | 21.74 | 242 | Red |
| 195 | | 2-55 | | | | |

TABLE 14

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.59 | 22.86 | 283 | Red |
| 196 | 1-41 | 2-1 | | | | |
| Example | | Compound | 3.58 | 23.30 | 297 | Red |
| 197 | | 2-20 | | | | |
| Example | | Compound | 3.56 | 24.04 | 303 | Red |
| 198 | | 2-29 | | | | |
| Example | | Compound | 3.59 | 23.84 | 279 | Red |
| 199 | | 2-37 | | | | |
| Example | | Compound | 3.55 | 23.39 | 294 | Red |
| 200 | | 2-51 | | | | |
| Example | Compound | Compound | 3.53 | 23.11 | 303 | Red |
| 201 | 1-42 | 2-2 | | | | |
| Example | | Compound | 3.56 | 23.65 | 280 | Red |
| 202 | | 2-23 | | | | |
| Example | | Compound | 3.55 | 23.39 | 302 | Red |
| 203 | | 2-30 | | | | |
| Example | | Compound | 3.57 | 23.99 | 294 | Red |
| 204 | | 2-38 | | | | |
| Example | | Compound | 3.55 | 22.96 | 283 | Red |
| 205 | | 2-52 | | | | |
| Example | Compound | Compound | 3.47 | 23.74 | 312 | Red |
| 206 | 1-43 | 2-3 | | | | |
| Example | | Compound | 3.44 | 24.43 | 342 | Red |
| 207 | | 2-16 | | | | |
| Example | | Compound | 3.43 | 24.23 | 324 | Red |
| 208 | | 2-31 | | | | |
| Example | | Compound | 3.40 | 24.36 | 331 | Red |
| 209 | | 2-39 | | | | |
| Example | | Compound | 3.42 | 24.01 | 304 | Red |
| 210 | | 2-53 | | | | |

TABLE 15

| | | | Driving | | | |
|---|---|---|---|---|---|---|
| | | | voltage | Efficiency | Lifetime | Luminescent |
| Category | First host | Second host | (V) | (cd/A) | T95(hr) | color |
| Example | Compound | Compound | 3.46 | 22.02 | 278 | Red |
| 211 | 1-44 | 2-4 | | | | |
| Example | | Compound | 3.45 | 22.69 | 242 | Red |
| 212 | | 2-21 | | | | |
| Example | | Compound | 3.51 | 22.87 | 270 | Red |
| 213 | | 2-32 | | | | |
| Example | | Compound | 3.45 | 22.59 | 263 | Red |
| 214 | | 2-40 | | | | |
| Example | | Compound | 3.47 | 22.26 | 268 | Red |
| 215 | | 2-54 | | | | |
| Example | Compound | Compound | 3.39 | 23.50 | 324 | Red |
| 216 | 1-45 | 2-5 | | | | |
| Example | | Compound | 3.42 | 23.68 | 331 | Red |
| 217 | | 2-19 | | | | |
| Example | | Compound | 3.48 | 23.77 | 333 | Red |
| 218 | | 2-33 | | | | |
| Example | | Compound | 3.39 | 24.19 | 334 | Red |
| 219 | | 2-41 | | | | |
| Example | | Compound | 3.46 | 24.48 | 297 | Red |
| 220 | | 2-55 | | | | |
| Example | Compound | Compound | 3.38 | 23.70 | 316 | Red |
| 221 | 1-46 | 2-6 | | | | |
| Example | | Compound | 3.39 | 23.98 | 310 | Red |
| 222 | | 2-17 | | | | |
| Example | | Compound | 3.44 | 24.53 | 327 | Red |
| 223 | | 2-22 | | | | |
| Example | | Compound | 3.43 | 24.12 | 322 | Red |
| 224 | | 2-42 | | | | |
| Example | | Compound | 3.40 | 23.97 | 304 | Red |
| 225 | | 2-56 | | | | |

TABLE 16

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.62 | 21.33 | 230 | Red |
| 226 | 1-47 | 2-7 | | | | |
| Example | | Compound | 3.63 | 21.20 | 252 | Red |
| 227 | | 2-35 | | | | |
| Example | | Compound | 3.60 | 21.46 | 240 | Red |
| 228 | | 2-2 | | | | |
| Example | | Compound | 3.59 | 21.72 | 245 | Red |
| 229 | | 2-43 | | | | |
| Example | | Compound | 3.60 | 21.65 | 229 | Red |
| 230 | | 2-57 | | | | |
| Example | Compound | Compound | 3.62 | 21.26 | 235 | Red |
| 231 | 1-48 | 2-8 | | | | |
| Example | | Compound | 3.60 | 21.84 | 238 | Red |
| 232 | | 2-36 | | | | |
| Example | | Compound | 3.61 | 21.56 | 239 | Red |
| 233 | | 2-65 | | | | |
| Example | | Compound | 3.59 | 21.97 | 233 | Red |
| 234 | | 2-44 | | | | |
| Example | | Compound | 3.63 | 21.48 | 253 | Red |
| 235 | | 2-58 | | | | |
| Example | Compound | Compound | 3.45 | 23.68 | 338 | Red |
| 236 | 1-49 | 2-9 | | | | |
| Example | | Compound | 3.44 | 24.29 | 331 | Red |
| 237 | | 2-18 | | | | |
| Example | | Compound | 3.42 | 24.56 | 362 | Red |
| 238 | | 2-22 | | | | |
| Example | | Compound | 3.43 | 23.72 | 307 | Red |
| 239 | | 2-45 | | | | |
| Example | | Compound | 3.39 | 24.00 | 335 | Red |
| 240 | | 2-66 | | | | |

TABLE 17

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.47 | 24.55 | 336 | Red |
| 241 | 1-50 D [symbol] | 2-10 | | | | |
| Example | | Compound | 3.46 | 24.35 | 297 | Red |
| 242 | | 2-24 | | | | |
| Example | | Compound | 3.41 | 24.47 | 331 | Red |
| 243 | | 2-72 | | | | |
| Example | | Compound | 3.46 | 23.98 | 324 | Red |
| 244 | | 2-59 | | | | |
| Example | | Compound | 3.47 | 23.60 | 338 | Red |
| 245 | | 2-65 | | | | |
| Example | Compound | Compound | 3.45 | 22.81 | 338 | Red |
| 246 | 1-51 | 2-11 | | | | |
| Example | D | Compound | 3.44 | 23.21 | 331 | Red |
| 247 | substitution | 2-25 | | | | |
| Example | | Compound | 3.38 | 23.56 | 325 | Red |
| 248 | | 2-67 | | | | |
| Example | | Compound | 3.43 | 23.43 | 307 | Red |
| 249 | | 2-46 | | | | |
| Example | | Compound | 3.39 | 23.26 | 335 | Red |
| 250 | | 2-60 | | | | |
| Example | Compound | Compound | 3.46 | 24.57 | 329 | Red |
| 251 | 1-52 | 2-12 | | | | |
| Example | | Compound | 3.48 | 24.08 | 326 | Red |
| 252 | | 2-26 | | | | |
| Example | | Compound | 3.37 | 24.46 | 328 | Red |
| 253 | | 2-71 | | | | |
| Example | | Compound | 3.44 | 23.52 | 336 | Red |
| 254 | | 2-47 | | | | |
| Example | | Compound | 3.46 | 23.77 | 327 | Red |
| 255 | | 2-61 | | | | |

TABLE 18

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.38 | 23.94 | 334 | Red |
| 256 | 1-53 | 2-13 | | | | |
| Example | | Compound | 3.48 | 23.89 | 342 | Red |
| 257 | | 2-27 | | | | |
| Example | | Compound | 3.44 | 24.42 | 316 | Red |
| 258 | | 2-68 | | | | |
| Example | | Compound | 3.40 | 24.52 | 325 | Red |
| 259 | | 2-48 | | | | |
| Example | | Compound | 3.40 | 24.32 | 307 | Red |
| 260 | | 2-62 | | | | |
| Example | Compound | Compound | 3.62 | 21.40 | 221 | Red |
| 261 | 1-54 | 2-14 | | | | |
| Example | | Compound | 3.59 | 21.93 | 221 | Red |
| 262 | | 2-28 | | | | |
| Example | | Compound | 3.61 | 21.92 | 253 | Red |
| 263 | | 2-69 | | | | |
| Example | | Compound | 3.60 | 21.18 | 236 | Red |
| 264 | | 2-49 | | | | |
| Example | | Compound | 3.63 | 21.11 | 254 | Red |
| 265 | | 2-63 | | | | |
| Example | Compound | Compound | 3.59 | 21.33 | 243 | Red |
| 266 | 1-55 | 2-15 | | | | |
| Example | | Compound | 3.61 | 21.53 | 232 | Red |
| 267 | | 2-29 | | | | |
| Example | | Compound | 3.62 | 21.59 | 231 | Red |
| 268 | | 2-70 | | | | |
| Example | | Compound | 3.62 | 21.58 | 233 | Red |
| 269 | | 2-50 | | | | |
| Example | | Compound | 3.59 | 21.27 | 245 | Red |
| 270 | | 2-64 | | | | |

TABLE 19

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.55 | 23.09 | 275 | Red |
| 271 | 1-56 | 2-4 | | | | |
| Example | | Compound | 3.54 | 22.90 | 293 | Red |
| 272 | | 2-21 | | | | |
| Example | | Compound | 3.58 | 23.74 | 290 | Red |
| 273 | | 2-32 | | | | |
| Example | | Compound | 3.57 | 23.63 | 290 | Red |
| 274 | | 2-40 | | | | |
| Example | | Compound | 3.58 | 23.71 | 277 | Red |
| 275 | | 2-54 | | | | |
| Example | Compound | Compound | 3.56 | 23.15 | 293 | Red |
| 276 | 1-57 | 2-5 | | | | |
| Example | | Compound | 3.59 | 23.36 | 289 | Red |
| 277 | | 2-19 | | | | |
| Example | | Compound | 3.55 | 23.02 | 282 | Red |
| 278 | | 2-33 | | | | |
| Example | | Compound | 3.55 | 23.05 | 305 | Red |
| 279 | | 2-41 | | | | |
| Example | | Compound | 3.52 | 22.85 | 282 | Red |
| 280 | | 2-55 | | | | |
| Example | Compound | Compound | 3.45 | 23.09 | 307 | Red |
| 281 | 1-58 | 2-6 | | | | |
| Example | | Compound | 3.44 | 22.90 | 299 | Red |
| 282 | | 2-17 | | | | |
| Example | | Compound | 3.48 | 23.74 | 322 | Red |
| 283 | | 2-34 | | | | |
| Example | | Compound | 3.38 | 23.63 | 309 | Red |
| 284 | | 2-42 | | | | |
| Example | | Compound | 3.42 | 23.71 | 318 | Red |
| 285 | | 2-56 | | | | |

TABLE 20

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.45 | 23.15 | 318 | Red |
| 286 | 1-59 | 2-7 | | | | |
| Example | | Compound | 3.42 | 23.36 | 305 | Red |
| 287 | | 2-35 | | | | |
| Example | | Compound | 3.45 | 23.02 | 304 | Red |
| 288 | | 2-2 | | | | |
| Example | | Compound | 3.48 | 23.05 | 327 | Red |
| 289 | | 2-43 | | | | |
| Example | | Compound | 3.40 | 22.85 | 306 | Red |
| 290 | | 2-57 | | | | |
| Example | Compound | Compound | 3.45 | 22.88 | 240 | Red |
| 291 | 1-60 | 2-8 | | | | |
| Example | | Compound | 3.46 | 22.02 | 280 | Red |
| 292 | | 2-36 | | | | |
| Example | | Compound | 3.49 | 22.81 | 283 | Red |
| 293 | | 2-65 | | | | |
| Example | | Compound | 3.45 | 22.58 | 279 | Red |
| 294 | | 2-44 | | | | |
| Example | | Compound | 3.52 | 22.80 | 284 | Red |
| 295 | | 2-58 | | | | |
| Example | Compound | Compound | 3.45 | 22.06 | 268 | Red |
| 296 | 1-61 | 2-1 | | | | |
| Example | | Compound | 3.49 | 22.03 | 276 | Red |
| 297 | | 2-20 | | | | |
| Example | | Compound | 3.51 | 22.21 | 254 | Red |
| 298 | | 2-29 | | | | |
| Example | | Compound | 3.54 | 22.70 | 271 | Red |
| 299 | | 2-37 | | | | |
| Example | | Compound | 3.53 | 22.27 | 251 | Red |
| 300 | | 2-51 | | | | |

TABLE 21

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.37 | 23.89 | 333 | Red |
| 301 | 1-62 | 2-2 | | | | |
| Example | | Compound | 3.37 | 24.51 | 319 | Red |
| 302 | | 2-23 | | | | |
| Example | | Compound | 3.38 | 24.36 | 321 | Red |
| 303 | | 2-30 | | | | |
| Example | | Compound | 3.47 | 24.58 | 308 | Red |
| 304 | | 2-38 | | | | |
| Example | | Compound | 3.40 | 23.50 | 326 | Red |
| 305 | | 2-52 | | | | |
| Example | Compound | Compound | 3.47 | 24.59 | 329 | Red |
| 306 | 1-63 | 2-3 | | | | |
| Example | | Compound | 3.46 | 24.27 | 305 | Red |
| 307 | | 2-16 | | | | |
| Example | | Compound | 3.43 | 23.85 | 321 | Red |
| 308 | | 2-31 | | | | |
| Example | | Compound | 3.40 | 23.88 | 312 | Red |
| 309 | | 2-39 | | | | |
| Example | | Compound | 3.47 | 23.90 | 330 | Red |
| 310 | | 2-53 | | | | |
| Example | Compound | Compound | 3.45 | 23.53 | 308 | Red |
| 311 | 1-64 | 2-4 | | | | |
| Example | | Compound | 3.41 | 24.49 | 318 | Red |
| 312 | | 2-21 | | | | |
| Example | | Compound | 3.37 | 23.95 | 311 | Red |
| 313 | | 2-32 | | | | |
| Example | | Compound | 3.42 | 24.11 | 309 | Red |
| 314 | | 2-40 | | | | |
| Example | | Compound | 3.40 | 24.21 | 302 | Red |
| 315 | | 2-54 | | | | |

TABLE 22

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.42 | 24.51 | 297 | Red |
| 316 | 1-65 | 2-5 | | | | |
| Example | | Compound | 3.48 | 24.50 | 321 | Red |
| 317 | | 2-19 | | | | |
| Example | | Compound | 3.41 | 23.76 | 301 | Red |
| 318 | | 2-33 | | | | |
| Example | | Compound | 3.39 | 23.90 | 340 | Red |
| 319 | | 2-41 | | | | |
| Example | | Compound | 3.38 | 24.35 | 337 | Red |
| 320 | | 2-55 | | | | |
| Example | Compound | Compound | 3.45 | 24.09 | 308 | Red |
| 321 | 1-66 | 2-6 | | | | |
| Example | | Compound | 3.41 | 23.95 | 318 | Red |
| 322 | | 2-17 | | | | |
| Example | | Compound | 3.37 | 23.18 | 311 | Red |
| 323 | | 2-34 | | | | |
| Example | | Compound | 3.42 | 23.35 | 309 | Red |
| 324 | | 2-42 | | | | |
| Example | | Compound | 3.40 | 23.12 | 302 | Red |
| 325 | | 2-56 | | | | |
| Example | Compound | Compound | 3.42 | 24.03 | 297 | Red |
| 326 | 1-67 | 2-7 | | | | |
| Example | | Compound | 3.48 | 23.36 | 321 | Red |
| 327 | | 2-35 | | | | |
| Example | | Compound | 3.41 | 24.09 | 301 | Red |
| 328 | | 2-2 | | | | |
| Example | | Compound | 3.39 | 23.95 | 340 | Red |
| 329 | | 2-43 | | | | |
| Example | | Compound | 3.38 | 23.86 | 337 | Red |
| 330 | | 2-57 | | | | |

TABLE 23

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.54 | 22.68 | 235 | Red |
| 331 | 1-68 | 2-8 | | | | |
| Example | | Compound | 3.61 | 22.05 | 240 | Red |
| 332 | | 2-36 | | | | |
| Example | | Compound | 3.61 | 22.01 | 251 | Red |
| 333 | | 2-65 | | | | |
| Example | | Compound | 3.57 | 22.89 | 244 | Red |
| 334 | | 2-44 | | | | |
| Example | | Compound | 3.58 | 22.57 | 254 | Red |
| 335 | | 2-58 | | | | |
| Example | Compound | Compound | 3.55 | 22.40 | 230 | Red |
| 336 | 1-69 | 2-9 | | | | |
| Example | | Compound | 3.56 | 23.04 | 247 | Red |
| 337 | | 2-18 | | | | |
| Example | | Compound | 3.59 | 22.81 | 237 | Red |
| 338 | | 2-22 | | | | |
| Example | | Compound | 3.52 | 22.26 | 255 | Red |
| 339 | | 2-45 | | | | |
| Example | | Compound | 3.55 | 22.28 | 236 | Red |
| 340 | | 2-66 | | | | |
| Example | Compound | Compound | 3.63 | 21.10 | 235 | Red |
| 341 | 1-70 | 2-10 | | | | |
| Example | | Compound | 3.63 | 21.24 | 240 | Red |
| 342 | | 2-24 | | | | |
| Example | | Compound | 3.60 | 21.09 | 251 | Red |
| 343 | | 2-72 | | | | |
| Example | | Compound | 3.62 | 21.49 | 244 | Red |
| 344 | | 2-59 | | | | |
| Example | | Compound | 3.61 | 21.09 | 254 | Red |
| 345 | | 2-65 | | | | |

TABLE 24

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.63 | 21.75 | 230 | Red |
| 346 | 1-71 | 2-11 | | | | |
| Example | | Compound | 3.63 | 21.73 | 247 | Red |
| 347 | | 2-25 | | | | |
| Example | | Compound | 3.63 | 21.17 | 237 | Red |
| 348 | | 2-67 | | | | |
| Example | | Compound | 3.62 | 21.98 | 255 | Red |
| 349 | | 2-46 | | | | |
| Example | | Compound | 3.63 | 21.40 | 236 | Red |
| 350 | | 2-60 | | | | |
| Example | Compound | Compound | 3.54 | 24.09 | 300 | Red |
| 351 | 1-72 | 2-12 | | | | |
| Example | | Compound | 3.61 | 23.95 | 299 | Red |
| 352 | | 2-26 | | | | |
| Example | | Compound | 3.61 | 23.18 | 293 | Red |
| 353 | | 2-71 | | | | |
| Example | | Compound | 3.57 | 23.35 | 294 | Red |
| 354 | | 2-47 | | | | |
| Example | | Compound | 3.58 | 23.12 | 301 | Red |
| 355 | | 2-61 | | | | |
| Example | Compound | Compound | 3.55 | 24.03 | 289 | Red |
| 356 | 1-73 | 2-13 | | | | |
| Example | | Compound | 3.56 | 23.36 | 305 | Red |
| 357 | | 2-27 | | | | |
| Example | | Compound | 3.59 | 24.09 | 294 | Red |
| 358 | | 2-68 | | | | |
| Example | | Compound | 3.52 | 23.95 | 287 | Red |
| 359 | | 2-48 | | | | |
| Example | | Compound | 3.55 | 23.86 | 304 | Red |
| 360 | | 2-62 | | | | |

TABLE 25

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.47 | 23.16 | 312 | Red |
| 361 | 1-74 | 2-14 | | | | |
| Example | | Compound | 3.42 | 23.11 | 327 | Red |
| 362 | | 2-28 | | | | |
| Example | | Compound | 3.46 | 23.69 | 311 | Red |
| 363 | | 2-69 | | | | |
| Example | | Compound | 3.47 | 23.26 | 324 | Red |
| 364 | | 2-49 | | | | |
| Example | | Compound | 3.44 | 23.50 | 331 | Red |
| 365 | | 2-63 | | | | |
| Example | Compound | Compound | 3.41 | 23.54 | 336 | Red |
| 366 | 1-75 | 2-15 | | | | |
| Example | | Compound | 3.48 | 23.73 | 335 | Red |
| 367 | | 2-29 | | | | |
| Example | | Compound | 3.39 | 23.57 | 310 | Red |
| 368 | | 2-70 | | | | |
| Example | | Compound | 3.47 | 22.84 | 314 | Red |
| 369 | | 2-50 | | | | |
| Example | | Compound | 3.39 | 22.83 | 311 | Red |
| 370 | | 2-64 | | | | |
| Example | Compound | Compound | 3.48 | 22.69 | 277 | Red |
| 371 | 1-76 | 2-1 | | | | |
| Example | | Compound | 3.46 | 22.66 | 273 | Red |
| 372 | | 2-20 | | | | |
| Example | | Compound | 3.50 | 22.12 | 256 | Red |
| 373 | | 2-29 | | | | |
| Example | | Compound | 3.53 | 22.07 | 254 | Red |
| 374 | | 2-37 | | | | |
| Example | | Compound | 3.49 | 22.39 | 242 | Red |
| 375 | | 2-51 | | | | |

TABLE 26

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Example | Compound | Compound | 3.53 | 22.49 | 255 | Red |
| 376 | 1-77 | 2-2 | | | | |
| Example | | Compound | 3.47 | 22.45 | 254 | Red |
| 377 | | 2-23 | | | | |
| Example | | Compound | 3.50 | 22.12 | 260 | Red |
| 378 | | 2-30 | | | | |
| Example | | Compound | 3.48 | 22.68 | 260 | Red |
| 379 | | 2-38 | | | | |
| Example | | Compound | 3.50 | 22.41 | 254 | Red |
| 380 | | 2-52 | | | | |
| Example | Compound | Compound | 3.38 | 24.40 | 295 | Red |
| 381 | 1-78 | 2-3 | | | | |
| Example | | Compound | 3.45 | 24.37 | 310 | Red |
| 382 | | 2-16 | | | | |
| Example | | Compound | 3.41 | 24.26 | 320 | Red |
| 383 | | 2-31 | | | | |
| Example | | Compound | 3.42 | 24.12 | 331 | Red |
| 384 | | 2-39 | | | | |
| Example | | Compound | 3.43 | 23.58 | 322 | Red |
| 385 | | 2-53 | | | | |
| Example | Compound | Compound | 3.47 | 23.65 | 302 | Red |
| 386 | 1-79 | 2-4 | | | | |
| Example | | Compound | 3.48 | 23.86 | 308 | Red |
| 387 | | 2-21 | | | | |
| Example | | Compound | 3.38 | 24.05 | 301 | Red |
| 388 | | 2-32 | | | | |
| Example | | Compound | 3.42 | 24.51 | 321 | Red |
| 389 | | 2-40 | | | | |
| Example | | Compound | 3.37 | 24.37 | 310 | Red |
| 390 | | 2-54 | | | | |

TABLE 27

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.92 | 17.33 | 169 | Red |
| Example 1 | A-1 | 2-1 | | | | |
| Comparative | | Compound | 3.92 | 17.65 | 169 | Red |
| Example 2 | | 2-20 | | | | |
| Comparative | | Compound | 3.91 | 17.60 | 171 | Red |
| Example 3 | | 2-29 | | | | |
| Comparative | | Compound | 3.95 | 17.61 | 170 | Red |
| Example 4 | | 2-37 | | | | |
| Comparative | | Compound | 3.92 | 18.09 | 173 | Red |
| Example 5 | | 2-51 | | | | |
| Comparative | Compound | Compound | 3.93 | 18.09 | 179 | Red |
| Example 6 | A-2 | 2-2 | | | | |
| Comparative | | Compound | 3.93 | 17.96 | 161 | Red |
| Example 7 | | 2-23 | | | | |
| Comparative | | Compound | 3.91 | 17.81 | 178 | Red |
| Example 8 | | 2-30 | | | | |
| Comparative | | Compound | 3.88 | 17.52 | 176 | Red |
| Example 9 | | 2-38 | | | | |
| Comparative | | Compound | 3.94 | 17.67 | 167 | Red |
| Example 10 | | 2-52 | | | | |
| Comparative | Compound | Compound | 4.15 | 15.65 | 133 | Red |
| Example 11 | A-3 | 2-3 | | | | |
| Comparative | | Compound | 4.12 | 15.31 | 135 | Red |
| Example 12 | | 2-16 | | | | |
| Comparative | | Compound | 4.06 | 14.80 | 113 | Red |
| Example 13 | | 2-31 | | | | |
| Comparative | | Compound | 4.17 | 15.45 | 127 | Red |
| Example 14 | | 2-39 | | | | |
| Comparative | | Compound | 4.05 | 16.04 | 128 | Red |
| Example 15 | | 2-53 | | | | |

TABLE 28

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 4.05 | 15.44 | 131 | Red |
| Example 16 | A-4 | 2-4 | | | | |
| Comparative | | Compound | 4.12 | 14.86 | 133 | Red |
| Example 17 | | 2-21 | | | | |
| Comparative | | Compound | 4.08 | 15.59 | 133 | Red |
| Example 18 | | 2-32 | | | | |
| Comparative | | Compound | 4.11 | 15.91 | 118 | Red |
| Example 19 | | 2-40 | | | | |
| Comparative | | Compound | 4.11 | 14.59 | 114 | Red |
| Example 20 | | 2-54 | | | | |
| Comparative | Compound | Compound | 3.91 | 16.30 | 160 | Red |
| Example 21 | A-5 | 2-5 | | | | |
| Comparative | | Compound | 3.94 | 17.32 | 153 | Red |
| Example 22 | | 2-19 | | | | |
| Comparative | | Compound | 3.92 | 17.18 | 156 | Red |
| Example 23 | | 2-33 | | | | |
| Comparative | | Compound | 3.91 | 17.35 | 147 | Red |
| Example 24 | | 2-41 | | | | |
| Comparative | | Compound | 3.90 | 16.65 | 160 | Red |
| Example 25 | | 2-55 | | | | |
| Comparative | Compound | Compound | 3.93 | 16.64 | 152 | Red |
| Example 26 | A-6 | 2-6 | | | | |
| Comparative | | Compound | 3.94 | 17.10 | 161 | Red |
| Example 27 | | 2-17 | | | | |
| Comparative | | Compound | 3.90 | 16.67 | 145 | Red |
| Example 28 | | 2-34 | | | | |
| Comparative | | Compound | 3.92 | 17.33 | 144 | Red |
| Example 29 | | 2-42 | | | | |
| Comparative | | Compound | 3.91 | 16.40 | 161 | Red |
| Example 30 | | 2-56 | | | | |

TABLE 29

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 4.19 | 15.65 | 87 | Red |
| Example 31 | A-7 | 2-7 | | | | |
| Comparative | | Compound | 4.09 | 15.31 | 112 | Red |
| Example 32 | | 2-35 | | | | |
| Comparative | | Compound | 4.18 | 14.80 | 107 | Red |
| Example 33 | | 2-2 | | | | |
| Comparative | | Compound | 4.17 | 15.45 | 87 | Red |
| Example 34 | | 2-49 | | | | |
| Comparative | | Compound | 4.14 | 16.04 | 112 | Red |
| Example 35 | | 2-57 | | | | |
| Comparative | Compound | Compound | 4.14 | 15.44 | 108 | Red |
| Example 36 | A-8 | 2-8 | | | | |
| Comparative | | Compound | 4.18 | 14.86 | 107 | Red |
| Example 37 | | 2-36 | | | | |
| Comparative | | Compound | 4.18 | 15.59 | 82 | Red |
| Example 38 | | 2-65 | | | | |
| Comparative | | Compound | 4.11 | 15.91 | 108 | Red |
| Example 39 | | 2-44 | | | | |
| Comparative | | Compound | 4.17 | 14.59 | 106 | Red |
| Example 40 | | 2-58 | | | | |
| Comparative | Compound | Compound | 4.16 | 15.50 | 112 | Red |
| Example 41 | A-9 | 2-9 | | | | |
| Comparative | | Compound | 4.06 | 15.52 | 114 | Red |
| Example 42 | | 2-18 | | | | |
| Comparative | | Compound | 4.17 | 15.86 | 133 | Red |
| Example 43 | | 2-22 | | | | |
| Comparative | | Compound | 4.10 | 15.46 | 126 | Red |
| Example 44 | | 2-45 | | | | |
| Comparative | | Compound | 4.11 | 15.59 | 120 | Red |
| Example 45 | | 2-66 | | | | |

TABLE 30

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.95 | 17.54 | 169 | Red |
| Example 46 | A-10 | 2-10 | | | | |
| Comparative | | Compound | 3.92 | 18.01 | 164 | Red |
| Example 47 | | 2-24 | | | | |
| Comparative | | Compound | 3.95 | 17.45 | 179 | Red |
| Example 48 | | 2-72 | | | | |
| Comparative | | Compound | 3.90 | 17.68 | 165 | Red |
| Example 49 | | 2-59 | | | | |
| Comparative | | Compound | 3.88 | 18.16 | 167 | Red |
| Example 50 | | 2-65 | | | | |
| Comparative | Compound | Compound | 3.90 | 17.34 | 165 | Red |
| Example 51 | A-11 | 2-11 | | | | |
| Comparative | | Compound | 3.95 | 17.67 | 174 | Red |
| Example 52 | | 2-25 | | | | |
| Comparative | | Compound | 3.95 | 17.86 | 163 | Red |
| Example 53 | | 2-67 | | | | |
| Comparative | | Compound | 3.90 | 17.94 | 163 | Red |
| Example 54 | | 2-46 | | | | |
| Comparative | | Compound | 3.88 | 17.59 | 172 | Red |
| Example 55 | | 2-60 | | | | |
| Comparative | Compound | Compound | 4.19 | 16.34 | 87 | Red |
| Example 56 | A-12 | 2-12 | | | | |
| Comparative | | Compound | 4.17 | 15.08 | 83 | Red |
| Example 57 | | 2-26 | | | | |
| Comparative | | Compound | 4.15 | 15.29 | 115 | Red |
| Example 58 | | 2-31 | | | | |
| Comparative | | Compound | 4.19 | 15.35 | 100 | Red |
| Example 59 | | 2-47 | | | | |
| Comparative | | Compound | 4.20 | 15.08 | 88 | Red |
| Example 60 | | 2-61 | | | | |

TABLE 31

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 4.13 | 16.31 | 155 | Red |
| Example 61 | 1-1 | B-1 | | | | |
| Comparative | Compound | | 4.05 | 15.32 | 125 | Red |
| Example 62 | 1-7 | | | | | |
| Comparative | Compound | | 4.11 | 15.60 | 132 | Red |
| Example 63 | 1-16 | | | | | |
| Comparative | Compound | | 4.14 | 15.38 | 129 | Red |
| Example 64 | 1-28 | | | | | |
| Comparative | Compound | | 4.13 | 14.59 | 125 | Red |
| Example 65 | 1-35 | | | | | |
| Comparative | Compound | | 4.14 | 15.98 | 168 | Red |
| Example 66 | 1-43 | | | | | |
| Comparative | Compound | | 4.11 | 16.26 | 123 | Red |
| Example 67 | 1-57 | | | | | |
| Comparative | Compound | | 4.17 | 16.08 | 153 | Red |
| Example 68 | 1-72 | | | | | |
| Comparative | Compound | Compound | 3.91 | 17.82 | 141 | Red |
| Example 69 | 1-2 | B-2 | | | | |
| Comparative | Compound | | 3.94 | 17.30 | 160 | Red |
| Example 70 | 1-10 | | | | | |
| Comparative | Compound | | 3.88 | 17.34 | 184 | Red |
| Example 71 | 1-19 | | | | | |
| Comparative | Compound | | 3.89 | 17.99 | 166 | Red |
| Example 72 | 1-26 | | | | | |
| Comparative | Compound | | 3.93 | 17.58 | 194 | Red |
| Example 73 | 1-31 | | | | | |
| Comparative | Compound | | 3.90 | 17.32 | 182 | Red |
| Example 74 | 1-54 | | | | | |
| Comparative | Compound | | 3.95 | 17.36 | 186 | Red |
| Example 75 | 1-66 | | | | | |
| Comparative | Compound | | 3.91 | 18.11 | 164 | Red |
| Example 76 | 1-78 | | | | | |

TABLE 32

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.91 | 17.34 | 163 | Red |
| Example 77 | 1-3 | B-3 | | | | |
| Comparative | Compound | | 3.89 | 17.43 | 197 | Red |
| Example 78 | 1-12 | | | | | |
| Comparative | Compound | | 3.89 | 16.56 | 183 | Red |
| Example 79 | 1-24 | | | | | |
| Comparative | Compound | | 3.89 | 17.09 | 144 | Red |
| Example 80 | 1-37 | | | | | |
| Comparative | Compound | | 3.93 | 16.45 | 180 | Red |
| Example 81 | 1-42 | | | | | |
| Comparative | Compound | | 3.88 | 17.40 | 186 | Red |
| Example 82 | 1-50 | | | | | |
| Comparative | Compound | | 3.91 | 17.29 | 175 | Red |
| Example 83 | 1-64 | | | | | |
| Comparative | Compound | | 3.90 | 16.52 | 156 | Red |
| Example 84 | 1-76 | | | | | |
| Comparative | Compound | Compound | 4.21 | 14.57 | 98 | Red |
| Example 85 | 1-4 | B-4 | | | | |
| Comparative | Compound | | 4.18 | 14.77 | 103 | Red |
| Example 86 | 1-11 | | | | | |
| Comparative | Compound | | 4.12 | 15.00 | 133 | Red |
| Example 87 | 1-23 | | | | | |
| Comparative | Compound | | 4.18 | 14.81 | 94 | Red |
| Example 88 | 1-36 | | | | | |
| Comparative | Compound | | 4.22 | 14.93 | 85 | Red |
| Example 89 | 1-44 | | | | | |
| Comparative | Compound | | 4.15 | 16.14 | 126 | Red |
| Example 90 | 1-53 | | | | | |
| Comparative | Compound | | 4.19 | 15.11 | 108 | Red |
| Example 91 | 1-69 | | | | | |
| Comparative | Compound | | 4.15 | 15.54 | 128 | Red |
| Example 92 | 1-75 | | | | | |

TABLE 33

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.89 | 18.06 | 154 | Red |
| Example 93 | 1-5 | B-5 | | | | |
| Comparative | Compound | | 3.94 | 17.50 | 167 | Red |
| Example 94 | 1-14 | | | | | |
| Comparative | Compound | | 3.89 | 17.49 | 187 | Red |
| Example 95 | 1-20 | | | | | |
| Comparative | Compound | | 3.93 | 17.88 | 195 | Red |
| Example 96 | 1-33 | | | | | |
| Comparative | Compound | | 3.91 | 17.35 | 180 | Red |
| Example 97 | 1-45 | | | | | |
| Comparative | Compound | | 3.90 | 18.18 | 177 | Red |
| Example 98 | 1-52 | | | | | |
| Comparative | Compound | | 3.94 | 17.72 | 189 | Red |
| Example 99 | 1-60 | | | | | |
| Comparative | Compound | | 3.88 | 17.40 | 162 | Red |
| Example 100 | 1-77 | | | | | |
| Comparative | Compound | Compound | 4.11 | 16.34 | 131 | Red |
| Example 101 | 1-6 | B-6 | | | | |
| Comparative | Compound | | 4.14 | 15.38 | 129 | Red |
| Example 102 | 1-13 | | | | | |
| Comparative | Compound | | 4.12 | 15.80 | 159 | Red |
| Example 103 | 1-21 | | | | | |
| Comparative | Compound | | 4.08 | 14.62 | 178 | Red |
| Example 104 | 1-32 | | | | | |
| Comparative | Compound | | 4.17 | 15.15 | 161 | Red |
| Example 105 | 1-40 | | | | | |
| Comparative | Compound | | 4.08 | 16.18 | 183 | Red |
| Example 106 | 1-51 | | | | | |
| Comparative | Compound | | 4.14 | 14.96 | 174 | Red |
| Example 107 | 1-67 | | | | | |
| Comparative | Compound | | 4.06 | 15.37 | 184 | Red |
| Example 108 | 1-79 | | | | | |

TABLE 34

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.91 | 17.94 | 191 | Red |
| Example 109 | 1-7 | B-7 | | | | |
| Comparative | Compound | | 3.95 | 17.98 | 160 | Red |
| Example 110 | 1-16 | | | | | |
| Comparative | Compound | | 3.90 | 17.36 | 184 | Red |
| Example 111 | 1-25 | | | | | |
| Comparative | Compound | | 3.94 | 17.30 | 195 | Red |
| Example 112 | 1-34 | | | | | |
| Comparative | Compound | | 3.88 | 17.36 | 203 | Red |
| Example 113 | 1-46 | | | | | |
| Comparative | Compound | | 3.91 | 17.76 | 161 | Red |
| Example 114 | 1-58 | | | | | |
| Comparative | Compound | | 3.91 | 17.61 | 175 | Red |
| Example 115 | 1-63 | | | | | |
| Comparative | Compound | | 3.89 | 17.93 | 206 | Red |
| Example 116 | 1-72 | | | | | |
| Comparative | Compound | Compound | 3.94 | 17.78 | 167 | Red |
| Example 117 | 1-8 | B-8 | | | | |
| Comparative | Compound | | 3.89 | 17.70 | 161 | Red |
| Example 118 | 1-17 | | | | | |
| Comparative | Compound | | 3.92 | 18.03 | 193 | Red |
| Example 119 | 1-29 | | | | | |
| Comparative | Compound | | 3.95 | 18.04 | 185 | Red |
| Example 120 | 1-38 | | | | | |
| Comparative | Compound | | 3.90 | 17.68 | 188 | Red |
| Example 121 | 1-48 | | | | | |
| Comparative | Compound | | 3.91 | 17.93 | 163 | Red |
| Example 122 | 1-55 | | | | | |
| Comparative | Compound | | 3.93 | 18.01 | 160 | Red |
| Example 123 | 1-62 | | | | | |
| Comparative | Compound | | 3.91 | 18.05 | 197 | Red |
| Example 124 | 1-73 | | | | | |

TABLE 35

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 4.10 | 16.03 | 122 | Red |
| Example 125 | 1-9 | B-9 | | | | |
| Comparative | Compound | | 4.09 | 16.32 | 183 | Red |
| Example 126 | 1-18 | | | | | |
| Comparative | Compound | | 4.05 | 15.46 | 173 | Red |
| Example 127 | 1-22 | | | | | |
| Comparative | Compound | | 4.12 | 15.14 | 185 | Red |
| Example 128 | 1-30 | | | | | |
| Comparative | Compound | | 4.05 | 14.64 | 177 | Red |
| Example 129 | 1-41 | | | | | |
| Comparative | Compound | | 4.09 | 16.22 | 134 | Red |
| Example 130 | 1-56 | | | | | |
| Comparative | Compound | | 4.06 | 15.96 | 179 | Red |
| Example 131 | 1-65 | | | | | |
| Comparative | Compound | | 4.12 | 16.35 | 188 | Red |
| Example 132 | 1-74 | | | | | |
| Comparative | Compound | Compound | 4.10 | 16.03 | 102 | Red |
| Example 133 | 1-1 | B-10 | | | | |
| Comparative | Compound | | 4.13 | 16.32 | 141 | Red |
| Example 134 | 1-15 | | | | | |
| Comparative | Compound | | 4.17 | 15.46 | 138 | Red |
| Example 135 | 1-26 | | | | | |
| Comparative | Compound | | 4.12 | 15.14 | 147 | Red |
| Example 136 | 1-35 | | | | | |
| Comparative | Compound | | 4.16 | 14.64 | 129 | Red |
| Example 137 | 1-49 | | | | | |
| Comparative | Compound | | 4.17 | 16.22 | 133 | Red |
| Example 138 | 1-59 | | | | | |
| Comparative | Compound | | 4.19 | 15.96 | 107 | Red |
| Example 139 | 1-68 | | | | | |
| Comparative | Compound | | 4.10 | 16.35 | 92 | Red |
| Example 140 | 1-71 | | | | | |

TABLE 36

| | | | Driving | | | |
|---|---|---|---|---|---|---|
| | First | Second | voltage | Efficiency | Lifetime | Luminescent |
| Category | host | host | (V) | (cd/A) | T95(hr) | color |
| Comparative | Compound | Compound | 3.94 | 18.00 | 169 | Red |
| Example 141 | 1-3 | B-11 | | | | |
| Comparative | Compound | | 3.95 | 17.88 | 153 | Red |
| Example 142 | 1-14 | | | | | |
| Comparative | Compound | | 3.92 | 17.65 | 197 | Red |
| Example 143 | 1-27 | | | | | |
| Comparative | Compound | | 3.91 | 18.00 | 167 | Red |
| Example 144 | 1-39 | | | | | |
| Comparative | Compound | | 3.88 | 17.47 | 164 | Red |
| Example 145 | 1-47 | | | | | |
| Comparative | Compound | | 3.90 | 17.65 | 193 | Red |
| Example 146 | 1-53 | | | | | |
| Comparative | Compound | | 3.90 | 17.38 | 176 | Red |
| Example 147 | 1-61 | | | | | |
| Comparative | Compound | | 3.93 | 17.46 | 165 | Red |
| Example 148 | 1-70 | | | | | |
| Comparative | Compound | Compound | 3.89 | 18.09 | 167 | Red |
| Example 149 | 1-7 | B-12 | | | | |
| Comparative | Compound | | 3.93 | 17.75 | 164 | Red |
| Example 150 | 1-16 | | | | | |
| Comparative | Compound | | 3.89 | 17.95 | 198 | Red |
| Example 151 | 1-25 | | | | | |
| Comparative | Compound | | 3.95 | 17.43 | 186 | Red |
| Example 152 | 1-34 | | | | | |
| Comparative | Compound | | 3.94 | 17.44 | 203 | Red |
| Example 153 | 1-46 | | | | | |
| Comparative | Compound | | 3.91 | 17.93 | 199 | Red |
| Example 154 | 1-58 | | | | | |
| Comparative | Compound | | 3.91 | 17.66 | 169 | Red |
| Example 155 | 1-63 | | | | | |
| Comparative | Compound | | 3.90 | 17.51 | 187 | Red |
| Example 156 | 1-72 | | | | | |

As a result of the experiment, it was found that the organic light emitting devices of Example, in which the compound of Chemical Formula 1 and the compound of Chemical Formula 2 according to the present disclosure were used in combination as a host for forming a light emitting layer, exhibited improved effects in terms of driving voltage, light emission efficiency, and lifetime characteristics.

On the other hand, the organic light emitting devices of Comparative Examples 1 to 60, in which Compounds A-1 to A-12 as a first host and the compound of Chemical Formula 2 of the present disclosure as a second host were co-deposited to form a light emitting layer, were increased in driving voltage and decreased in efficiency and lifetime characteristics as compared with Examples. Further, the organic light emitting devices of Comparative Examples 61 to 156, in which the compound of Chemical Formula 1 as the first host and Compounds B-1 to B-12 as the second host were co-deposited to form a light emitting layer, were also increased in driving voltage and decreased in efficiency and lifetime characteristics as compared with Examples.

These results are because, in the case of the organic light emitting devices of Examples in which the compound of Chemical Formula 1 and the compound of Chemical Formula 2 as the first and second hosts were used in combination in the formation of the light emitting layer, energy transfer to the dopant in the light emitting layer was well achieved, and electrons and holes were coupled in a more stable and balanced manner in the light emitting layer to form excitons.

From the above results, it can be confirmed that when the compound of Chemical Formula 1 and the compound of Chemical Formula 2 according to the present disclosure were used in combination as a host for forming a light emitting layer, the driving voltage, light emission efficiency, and lifetime characteristics of the organic light emitting devices can be greatly improved.

### DESCRIPTION OF REFERENCE NUMERALS

1: substrate
2: anode
3: light emitting layer
4: cathode
5: hole injection layer
6: hole transport layer
7: electron blocking layer
8: hole blocking layer
9: electron injection and transport layer

1. An organic light emitting device comprising:
an anode;
a cathode; and
a light emitting layer between the anode and the cathode,
wherein the light emitting layer comprises a compound represented by the following Chemical Formula 1 and a compound represented by the following Chemical Formula 2: 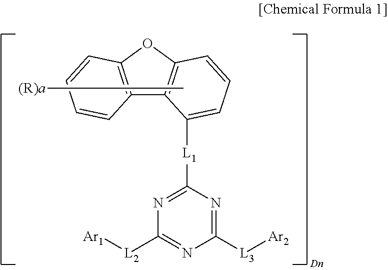
in Chemical Formula 1,
Ar₁ and Ar₂ are each independently a substituted or unsubstituted C₆₋₆₀ aryl,
L₁ to L₃ are each independently a single bond, or a substituted or unsubstituted C₆₋₆₀ arylene,
R is each independently hydrogen, deuterium, or a substituted or unsubstituted C₆₋₆₀ aryl,
Dn is a number of deuterium substitutions in the compound, where n is an integer of 0 or more, and
a is an integer of 0 to 7, 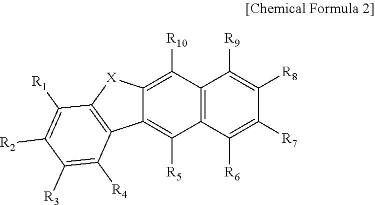
in Chemical Formula 2,
X is O or S,
one of R₁ to R₁₀ is represented by the following Chemical Formula 3, and the rest are each independently hydrogen or deuterium, 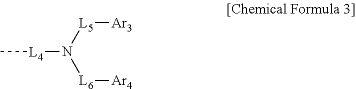
in Chemical Formula 3,
Ar₃ and Ar₄ are each independently a substituted or unsubstituted C₆₋₆₀ aryl; or a substituted or unsubstituted C₂₋₆₀ heteroaryl containing one or more selected from the group consisting of N, O and S,
L₄ is a single bond; a substituted or unsubstituted C₆₋₆₀ arylene; or a substituted or unsubstituted C₂₋₆ heteroarylene containing one or more selected from the group consisting of N, O and S, and
L₅ and L₆ are each independently a single bond; a substituted or unsubstituted C₆₋₆₀ arylene; or a substituted or unsubstituted C₂₋₆₀ heteroarylene containing one or more selected from the group consisting of N, O and S.

2. The organic light emitting device according to claim 1, wherein
the compound represented by Chemical Formula 1 is represented by one of the following Chemical Formulas 1-1 to 1-3: 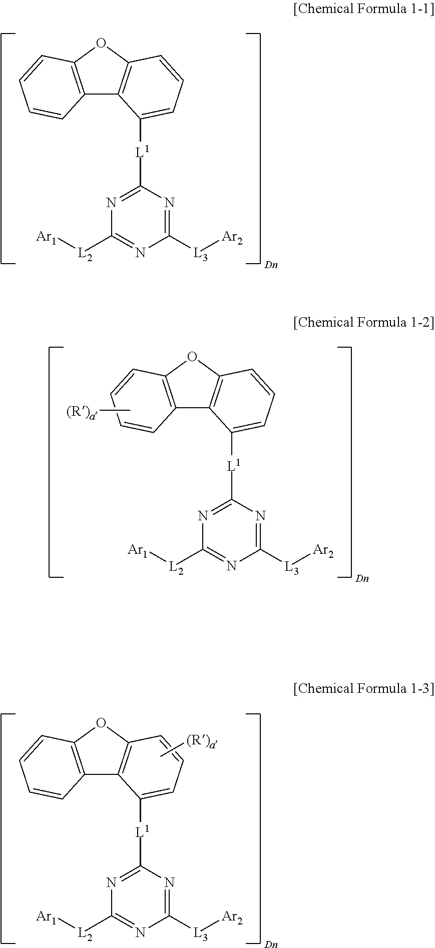
in Chemical Formulas 1-1 to 1-3,
Ar₁, Ar₂, L₁ to L₃, Dn and n are the same as defined in claim 1,
R′ is each independently deuterium, or a substituted or unsubstituted C₆₋₆₀ aryl,
a′ is an integer of 1 to 4, and
a″ is an integer of 1 to 3.

3. The organic light emitting device according to claim 1, wherein
Ar₁ and Ar₂ are each independently phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl,
each of which is unsubstituted or substituted with deuterium, phenyl or triphenylsilyl.

4. The organic light emitting device according to claim 1, wherein
L₁ to L₃ are each independently a single bond, phenylene, biphenyldiyl, naphthalenediyl, or binaphthalenediyl,
each of which, except for a single bond, is unsubstituted or substituted with deuterium, phenyl, or naphthyl.

5. The organic light emitting device according to claim 1, wherein
R is each independently hydrogen; deuterium; or phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, or fluoranthenyl, each of which, except for hydrogen and deuterium, is substituted or unsubstituted.

6. The organic light emitting device according to claim 1, wherein
a is 0 or 1.

7. The organic light emitting device according to claim 1, wherein
R is deuterium, or at least one of Ar₁, Ar₂, L₁ to L₃ and R is substituted with deuterium.

8. The organic light emitting device according to claim 1, wherein
the compound represented by Chemical Formula 1 contains 1 to 30 deuteriums.

9. The organic light emitting device according to claim 1, wherein
the compound represented by Chemical Formula 1 is one selected from the following:

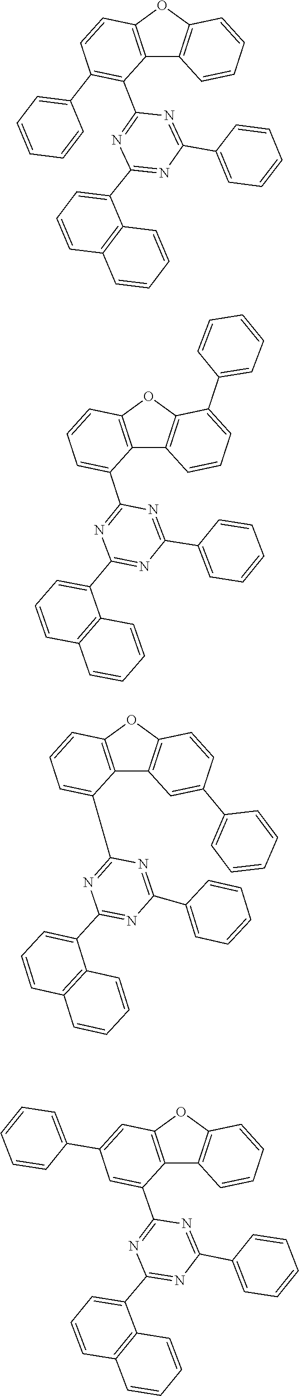

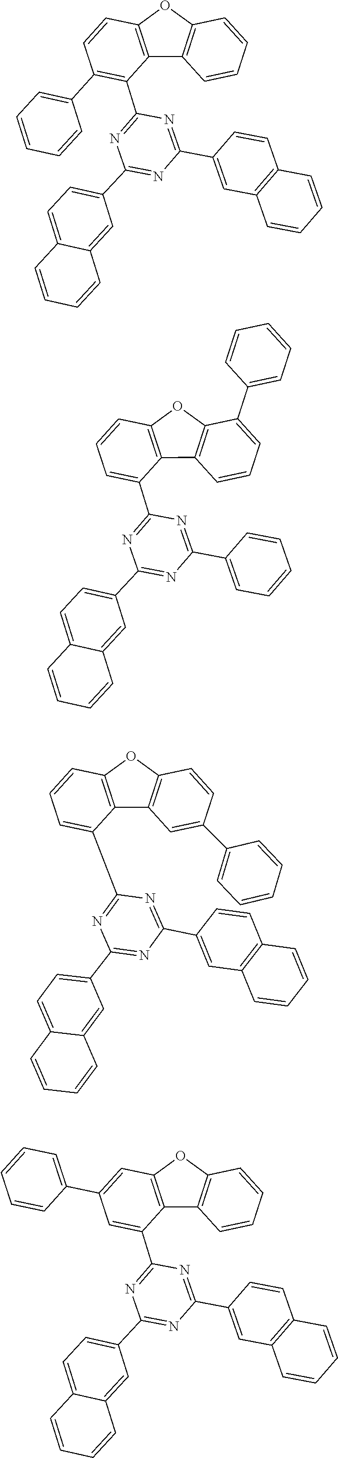 [SMILES: c1ccc(-c2ccc3oc4ccccc4c3c2-c2nc(-c3ccc4ccccc4c3)nc(-c3ccc4ccccc4c3)n2)cc1 ; c1ccc(-c2nc(-c3ccc4ccccc4c3)nc(-c3cccc4oc5c(-c6ccccc6)cccc5c34)n2)cc1 ; c1ccc(-c2ccc3oc4cccc(-c5nc(-c6ccc7ccccc7c6)nc(-c6ccc7ccccc7c6)n5)c4c3c2)cc1 ; c1ccc(-c2cc(-c3nc(-c4ccc5ccccc5c4)nc(-c4ccc5ccccc5c4)n3)c3c(c2)oc2ccccc23)cc1]

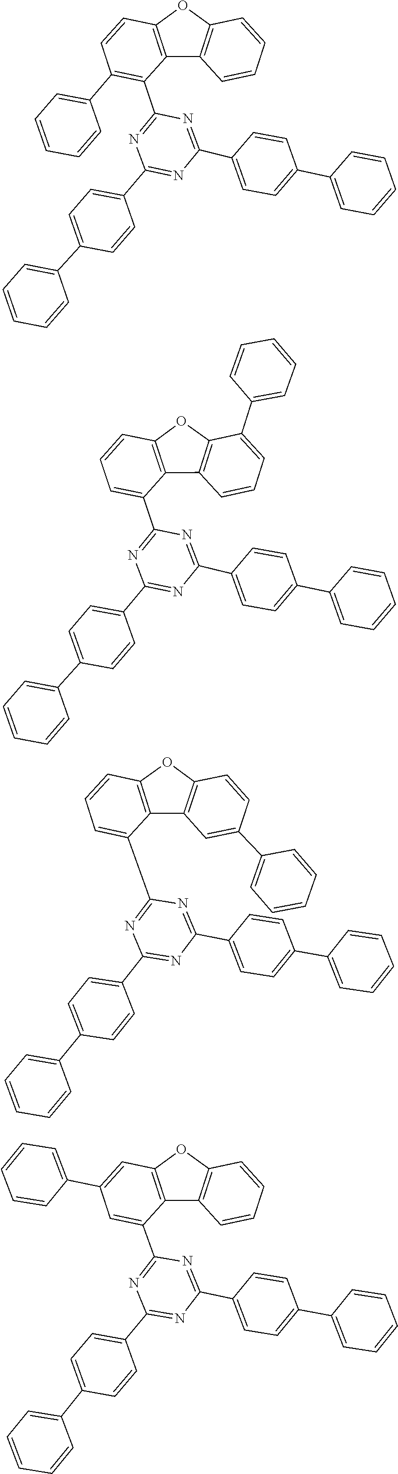

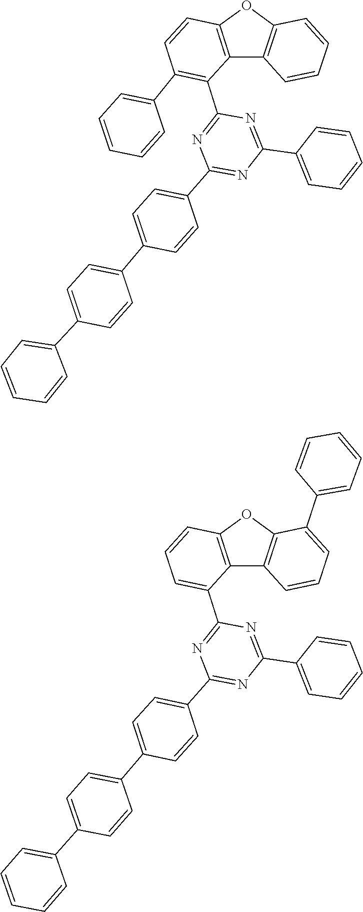 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1]

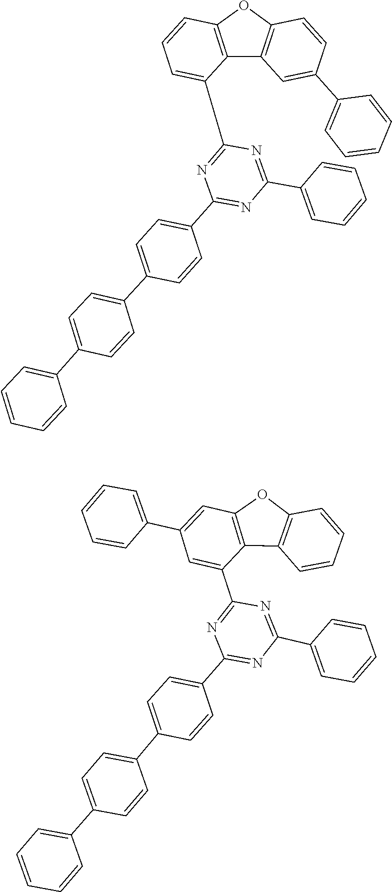 [SMILES: c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1]

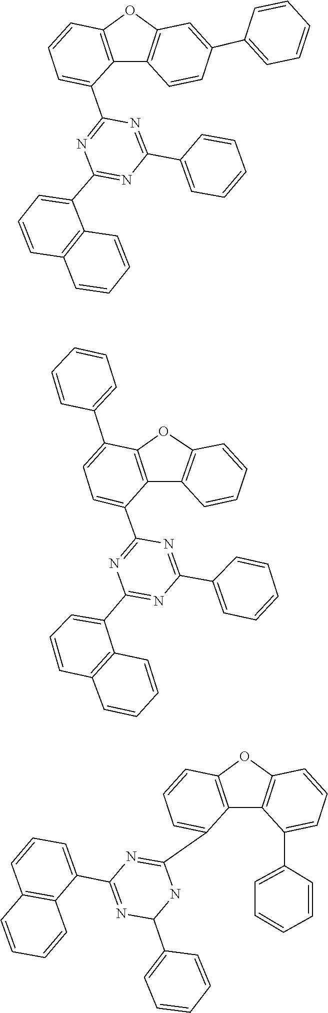

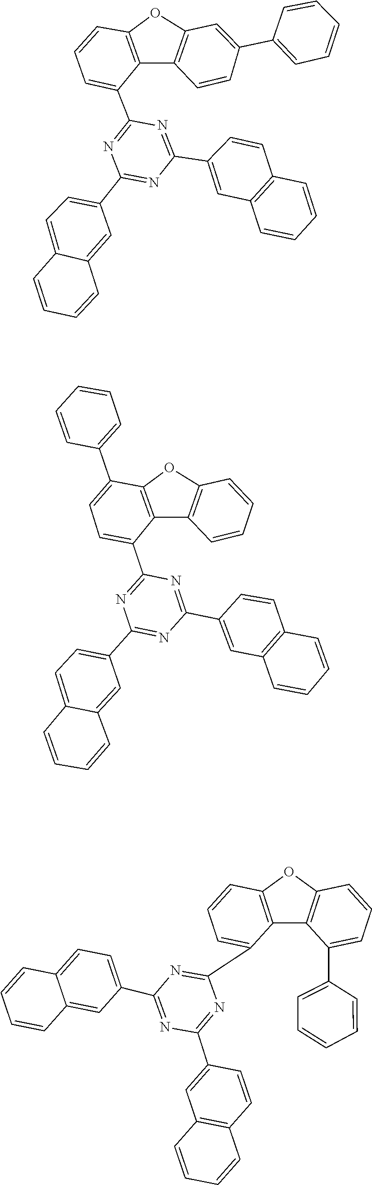 [SMILES: c1ccc(-c2ccc3c(c2)oc2cccc(-c4nc(-c5ccc6ccccc6c5)nc(-c5ccc6ccccc6c5)n4)c23)cc1 ; c1ccc(-c2ccc(-c3nc(-c4ccc5ccccc5c4)nc(-c4ccc5ccccc5c4)n3)c3c2oc2ccccc23)cc1 ; c1ccc(-c2cccc3oc4cccc(-c5nc(-c6ccc7ccccc7c6)nc(-c6ccc7ccccc7c6)n5)c4c23)cc1]

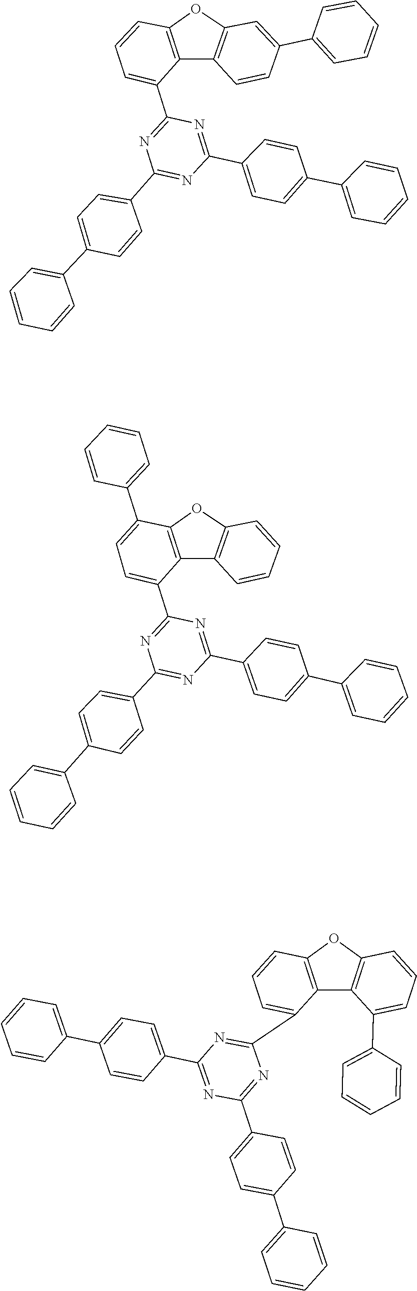 [SMILES: c1ccc(-c2ccc(-c3nc(-c4ccc(-c5ccccc5)cc4)nc(-c4cccc5oc6cc(-c7ccccc7)ccc6c45)n3)cc2)cc1 ; c1ccc(-c2ccc(-c3nc(-c4ccc(-c5ccccc5)cc4)nc(-c4ccc(-c5ccccc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccccc7)c6c45)n3)cc2)cc1]

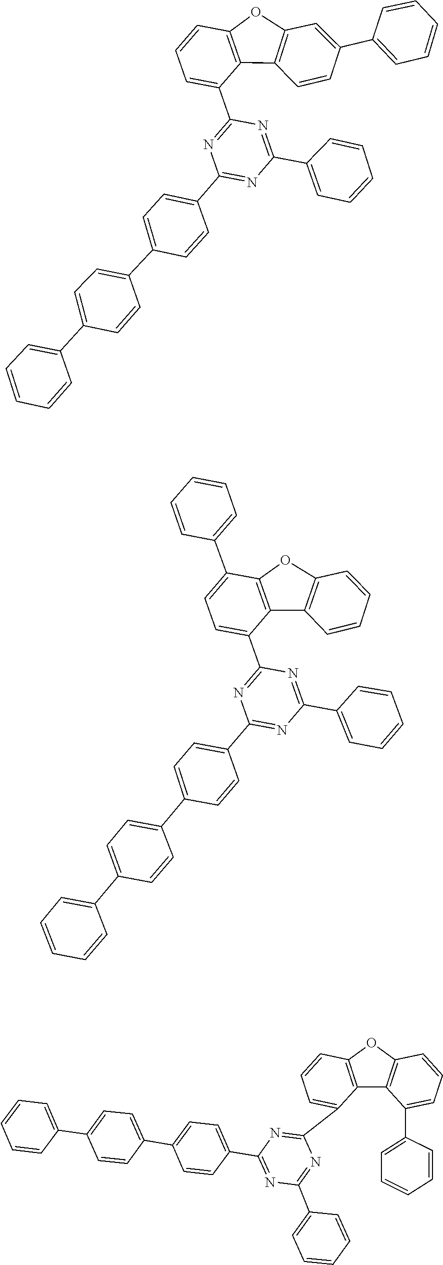 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

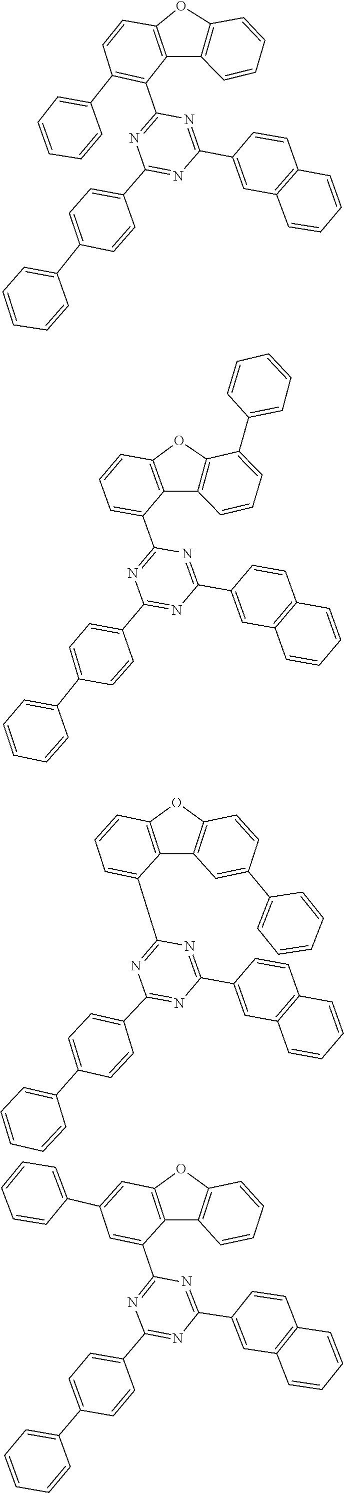

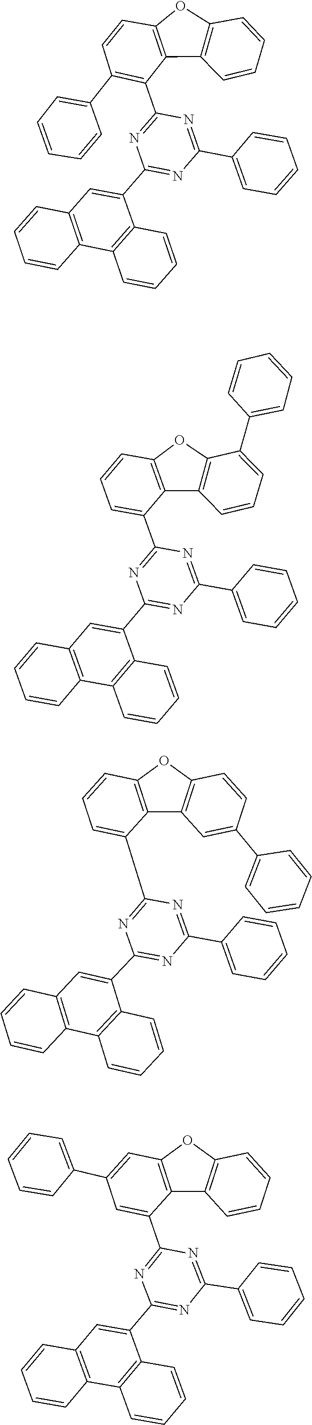

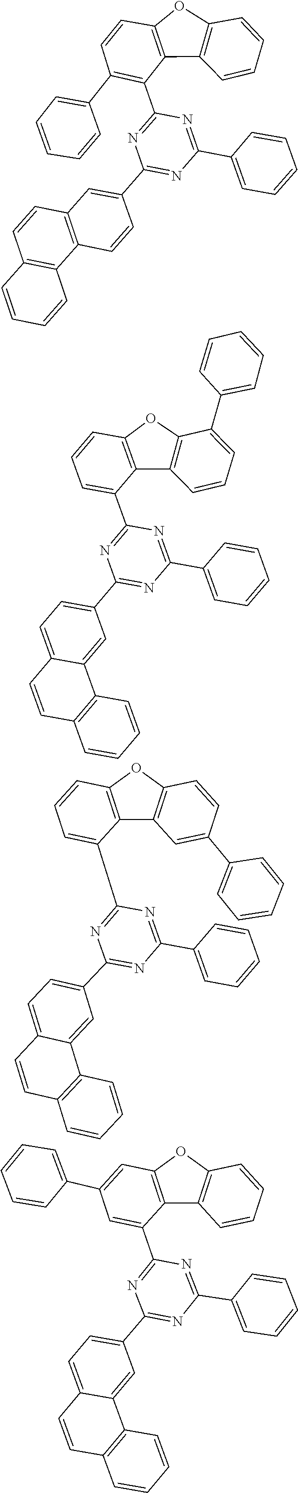

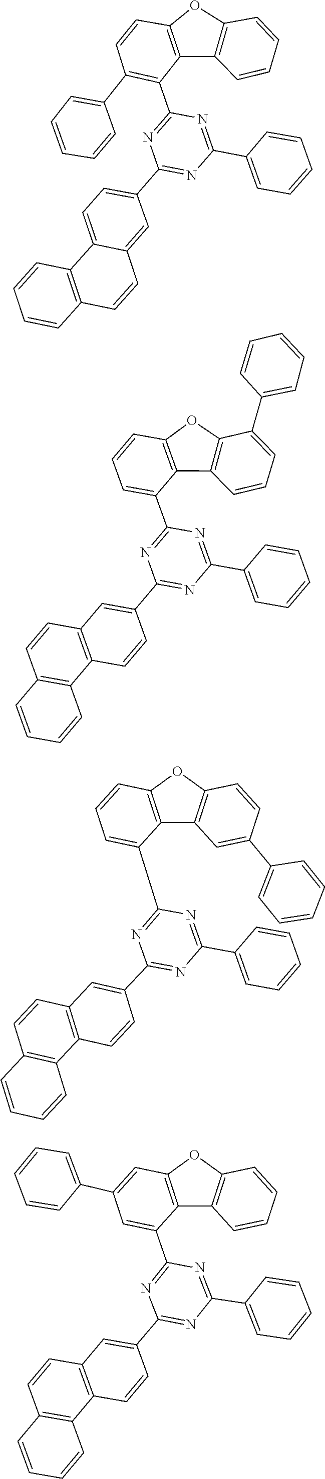

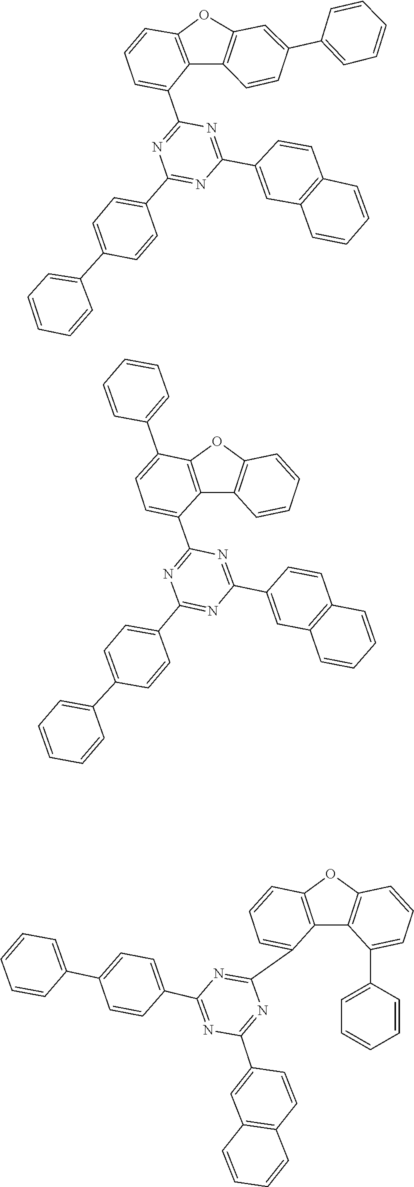 [SMILES: c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1]

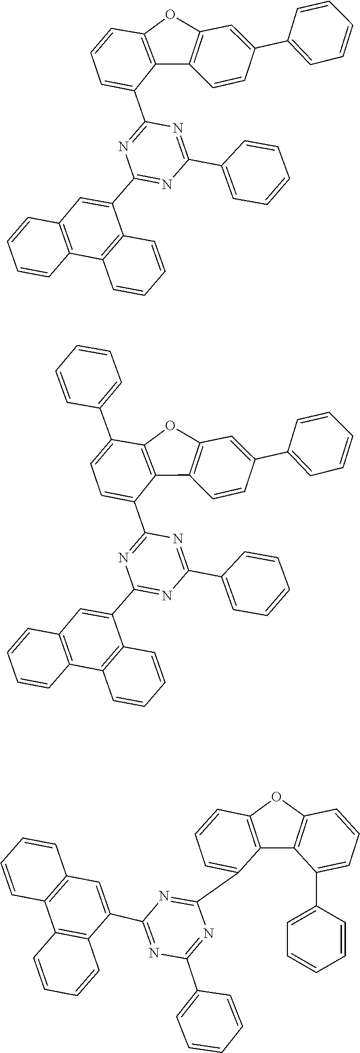 [SMILES: c1ccc(-c2ccc3c(c2)oc2cccc(-c4nc(-c5ccccc5)nc(-c5cc6ccccc6c6ccccc56)n4)c23)cc1 ; c1ccc(-c2ccc3c(c2)oc2c(-c4ccccc4)ccc(-c4nc(-c5ccccc5)nc(-c5cc6ccccc6c6ccccc56)n4)c23)cc1 ; c1ccc(-c2nc(-c3cc4ccccc4c4ccccc34)nc(-c3cccc4oc5cccc(-c6ccccc6)c5c34)n2)cc1]

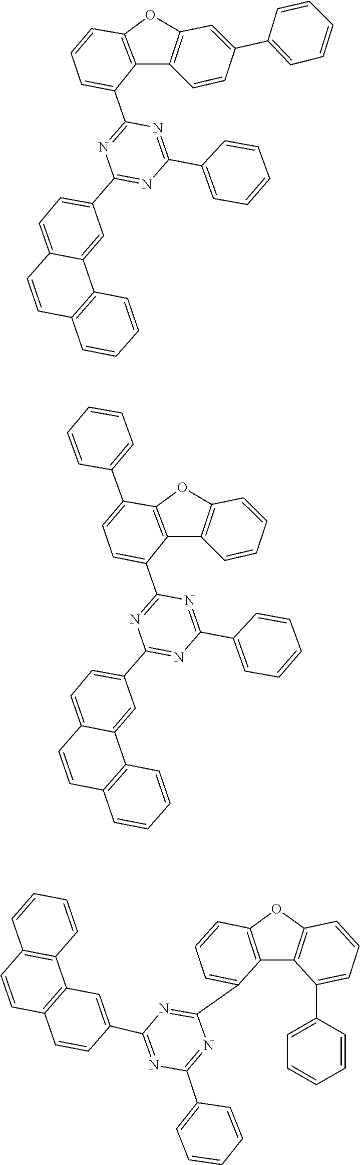 [SMILES: c1ccc(-c2ccc3c(c2)oc2cccc(-c4nc(-c5ccccc5)nc(-c5ccc6ccc7ccccc7c6c5)n4)c23)cc1 ; c1ccc(-c2nc(-c3ccc4ccc5ccccc5c4c3)nc(-c3ccc(-c4ccccc4)c4oc5ccccc5c34)n2)cc1 ; c1ccc(-c2nc(-c3ccc4ccc5ccccc5c4c3)nc(-c3cccc4oc5cccc(-c6ccccc6)c5c34)n2)cc1]

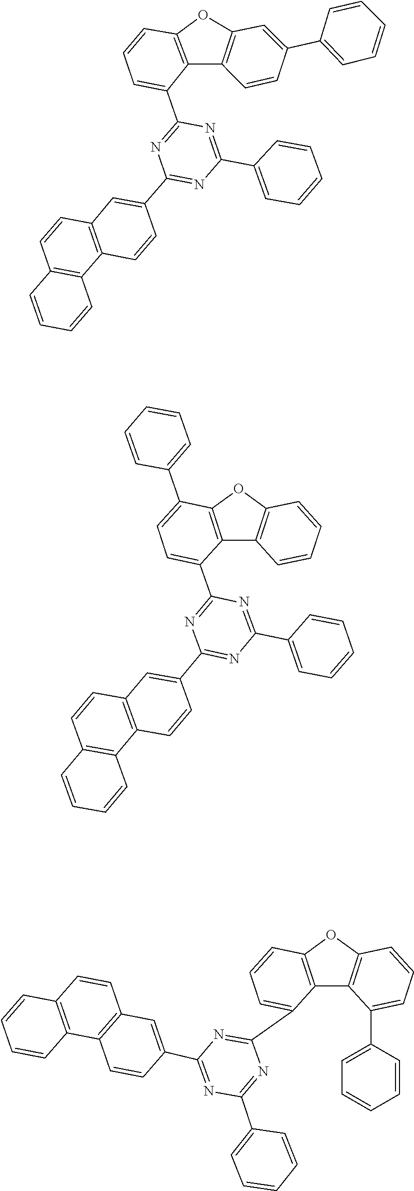 [SMILES: c1ccc(-c2ccc3c(c2)oc2cccc(-c4nc(-c5ccccc5)nc(-c5ccc6c(ccc7ccccc76)c5)n4)c23)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

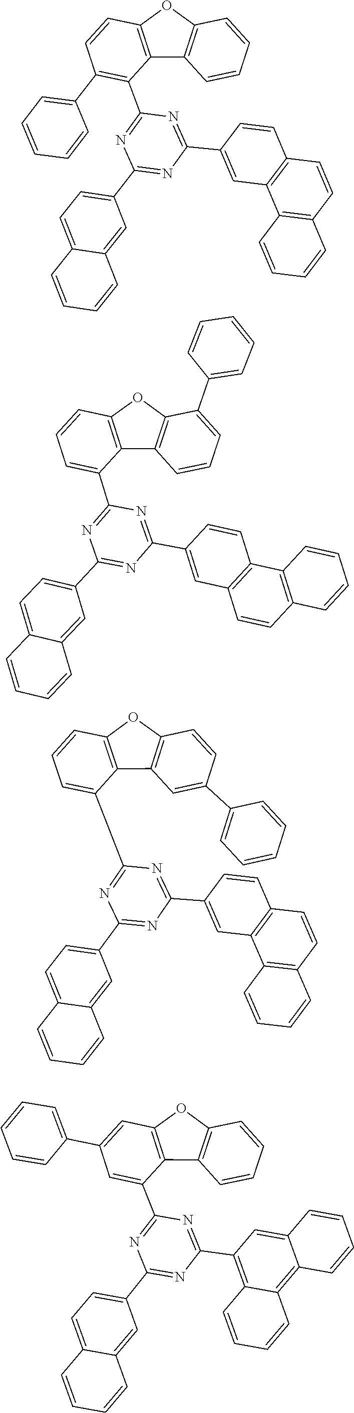

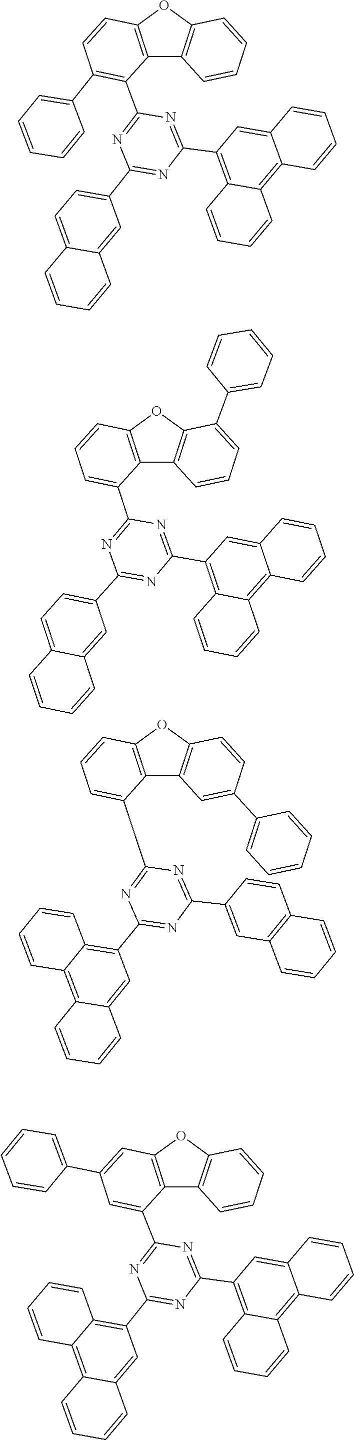

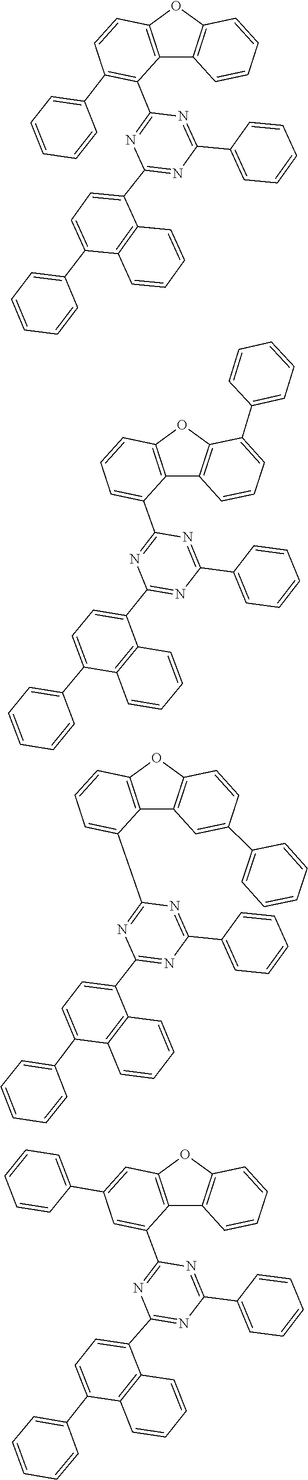

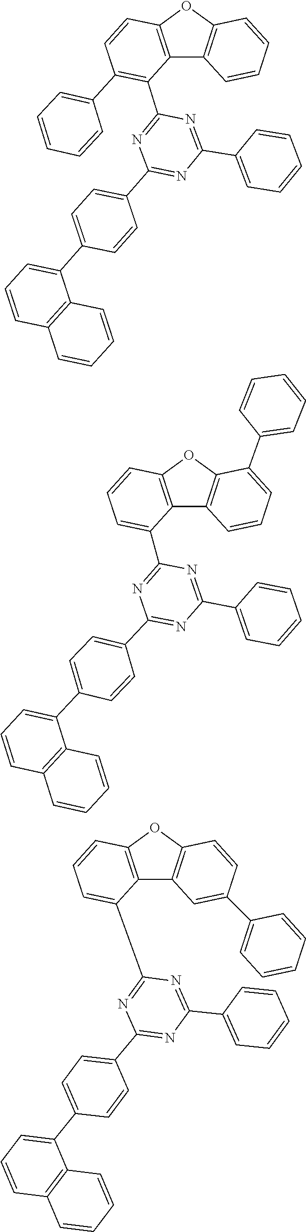

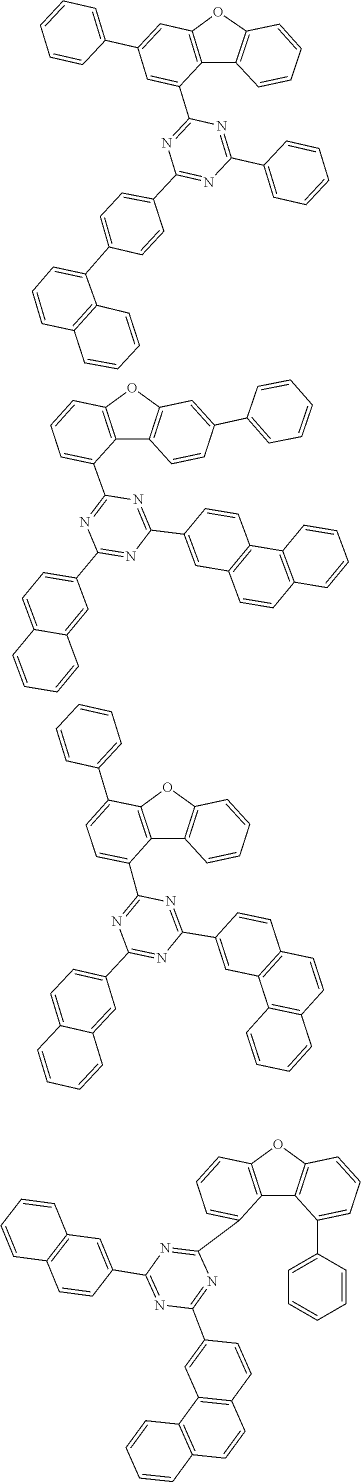

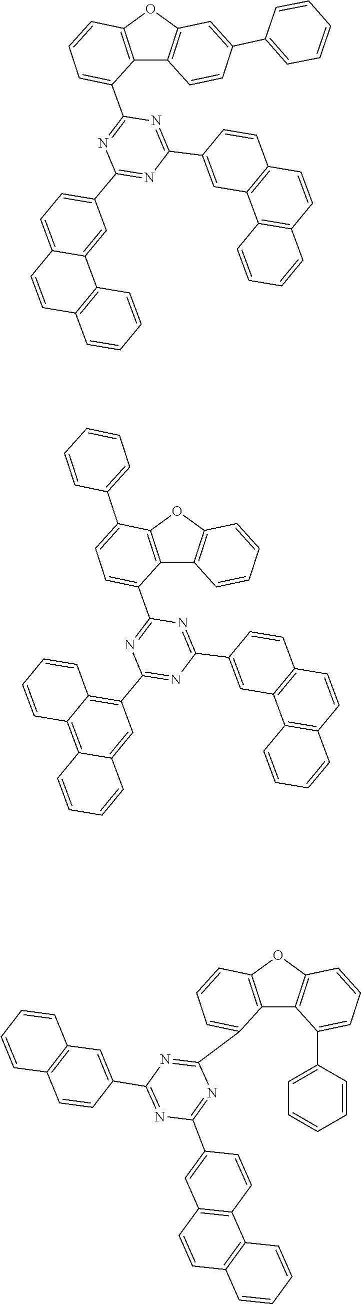 [SMILES: c1ccc(-c2ccc3c(c2)oc2cccc(-c4nc(-c5ccc6ccc7ccccc7c6c5)nc(-c5ccc6ccc7ccccc7c6c5)n4)c23)cc1 ; c1ccc(-c2ccc(-c3nc(-c4ccc5ccc6ccccc6c5c4)nc(-c4cc5ccccc5c5ccccc45)n3)c3c2oc2ccccc23)cc1 ; c1ccc(-c2cccc3oc4cccc(-c5nc(-c6ccc7ccccc7c6)nc(-c6ccc7c(ccc8ccccc87)c6)n5)c4c23)cc1]

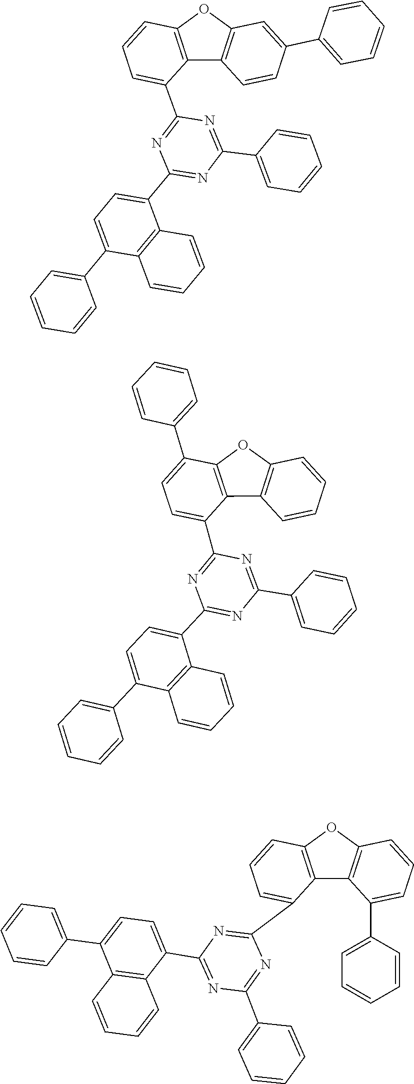 [SMILES: c1ccc(-c2ccc3c(c2)oc2cccc(-c4nc(-c5ccccc5)nc(-c5ccc(-c6ccccc6)c6ccccc56)n4)c23)cc1 ; c1ccc(-c2nc(-c3ccc(-c4ccccc4)c4ccccc34)nc(-c3ccc(-c4ccccc4)c4oc5ccccc5c34)n2)cc1 ; c1ccc(-c2nc(-c3ccc(-c4ccccc4)c4ccccc34)nc(-c3cccc4oc5cccc(-c6ccccc6)c5c34)n2)cc1]

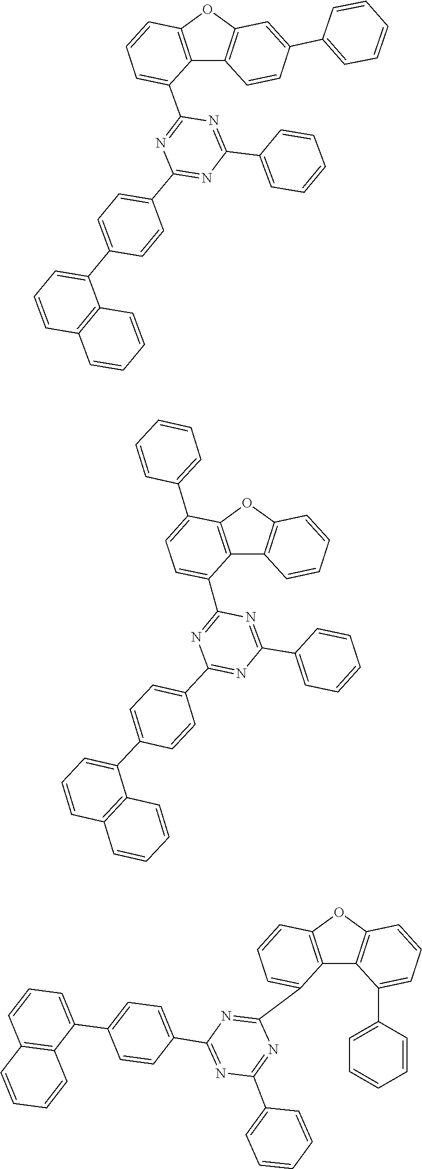 [SMILES: c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3ccc(-c4ccccc4)c4oc5ccccc5c34)n2)cc1 ; c1ccc(-c2nc(-c3ccc(-c4cccc5ccccc45)cc3)nc(-c3cccc4oc5cccc(-c6ccccc6)c5c34)n2)cc1]

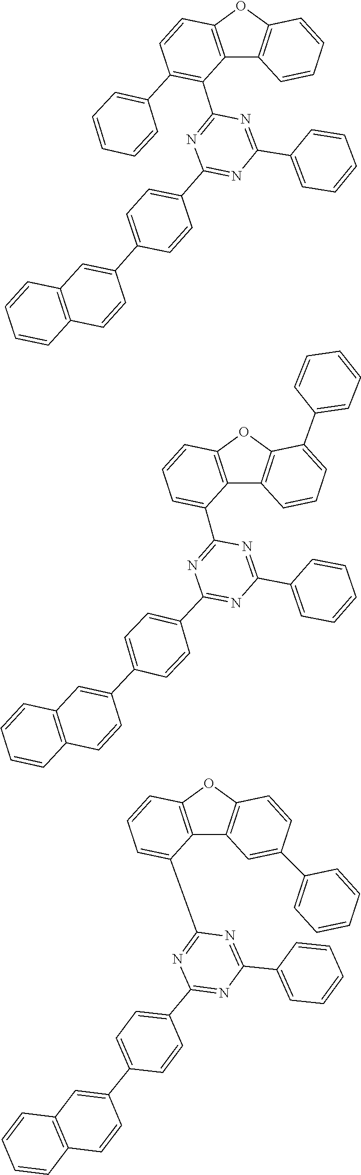 [SMILES: c1ccc(-c2nc(-c3ccc(-c4ccc5ccccc5c4)cc3)nc(-c3c(-c4ccccc4)ccc4oc5ccccc5c34)n2)cc1 ; c1ccc(-c2nc(-c3ccc(-c4ccc5ccccc5c4)cc3)nc(-c3cccc4oc5c(-c6ccccc6)cccc5c34)n2)cc1 ; c1ccc(-c2ccc3oc4cccc(-c5nc(-c6ccccc6)nc(-c6ccc(-c7ccc8ccccc8c7)cc6)n5)c4c3c2)cc1]

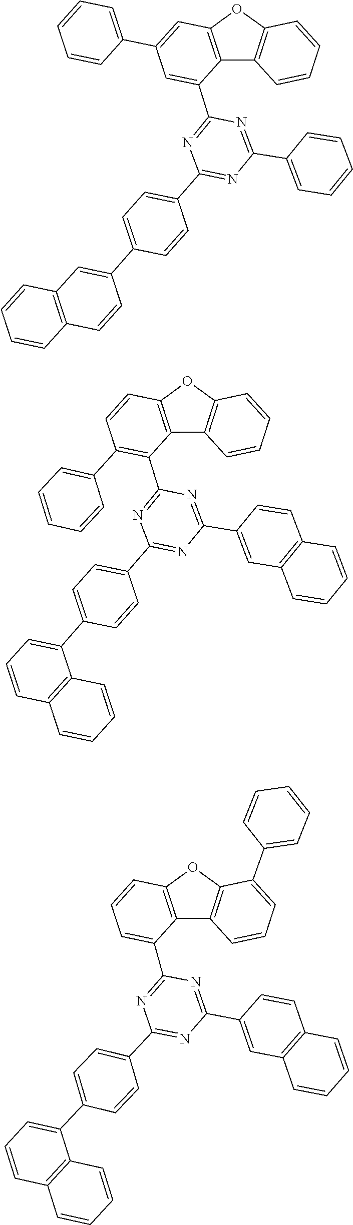 [SMILES: c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2ccc3oc4ccccc4c3c2-c2nc(-c3ccc(-c4cccc5ccccc45)cc3)nc(-c3ccc4ccccc4c3)n2)cc1 ; c1ccc(-c2cccc3c2oc2cccc(-c4nc(-c5ccc(-c6cccc7ccccc67)cc5)nc(-c5ccc6ccccc6c5)n4)c23)cc1]

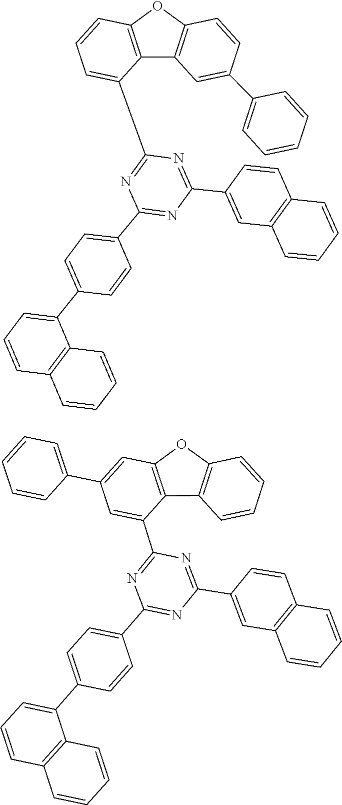

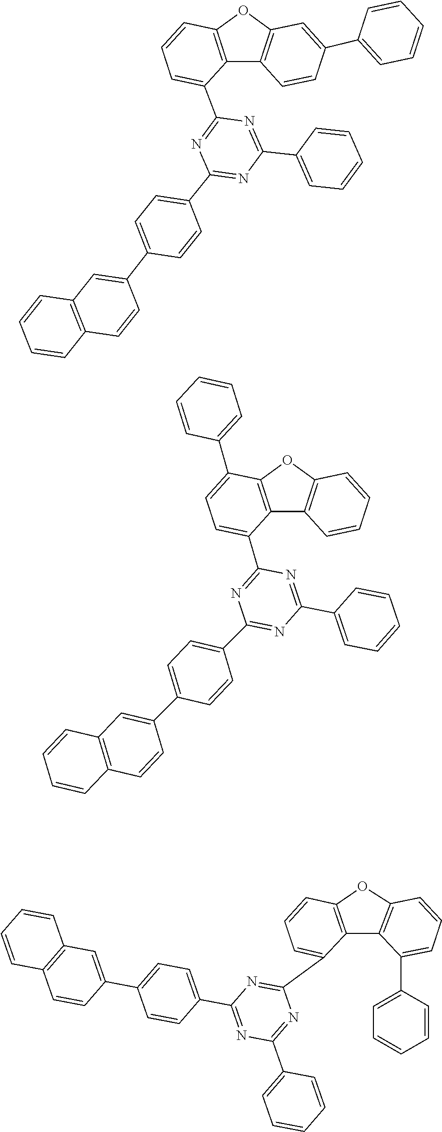 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2nc(-c3ccc(-c4ccc5ccccc5c4)cc3)nc(-c3ccc(-c4ccccc4)c4oc5ccccc5c34)n2)cc1 ; c1ccc(-c2nc(-c3ccc(-c4ccc5ccccc5c4)cc3)nc(-c3cccc4oc5cccc(-c6ccccc6)c5c34)n2)cc1]

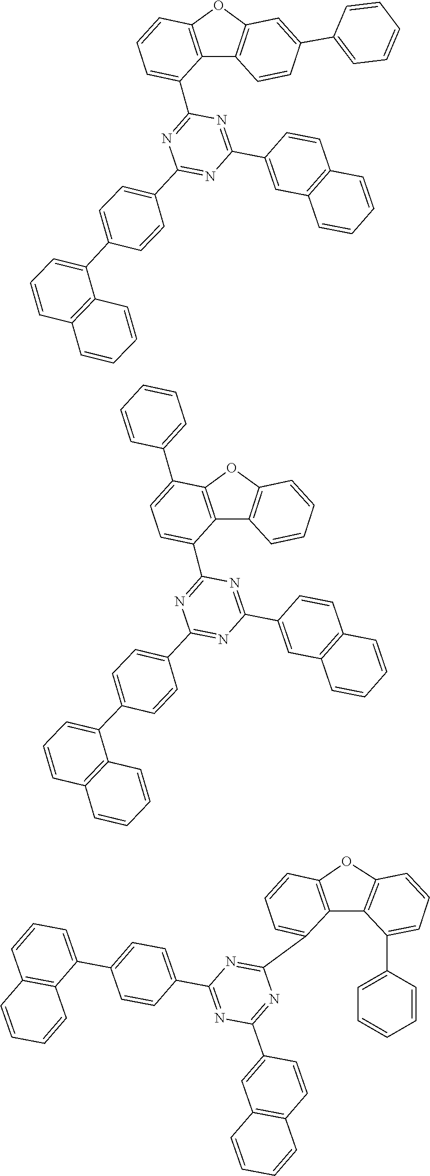

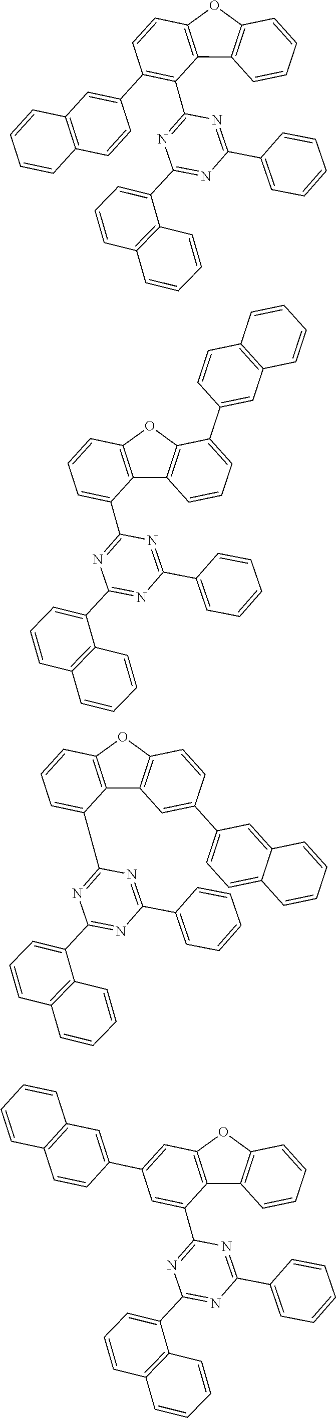

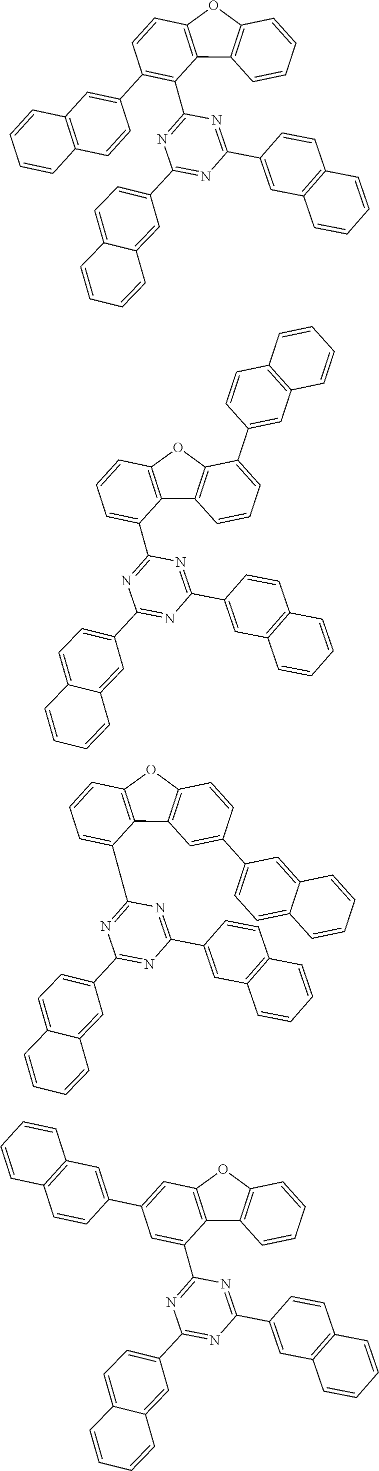

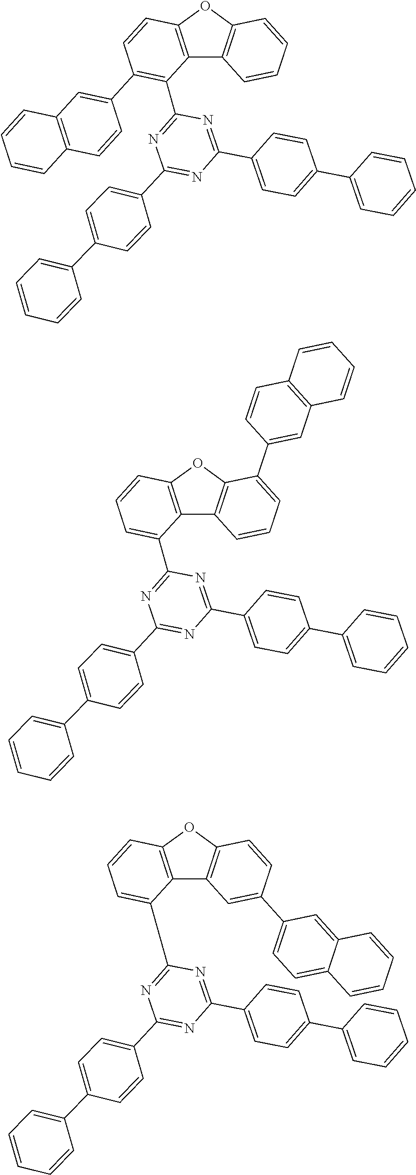 [SMILES: c1ccc(-c2ccc(-c3nc(-c4ccc(-c5ccccc5)cc4)nc(-c4c(-c5ccc6ccccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8ccccc8c7)cccc6c45)n3)cc2)cc1 ; c1ccc(-c2ccc(-c3nc(-c4ccc(-c5ccccc5)cc4)nc(-c4cccc5oc6ccc(-c7ccc8ccccc8c7)cc6c45)n3)cc2)cc1]

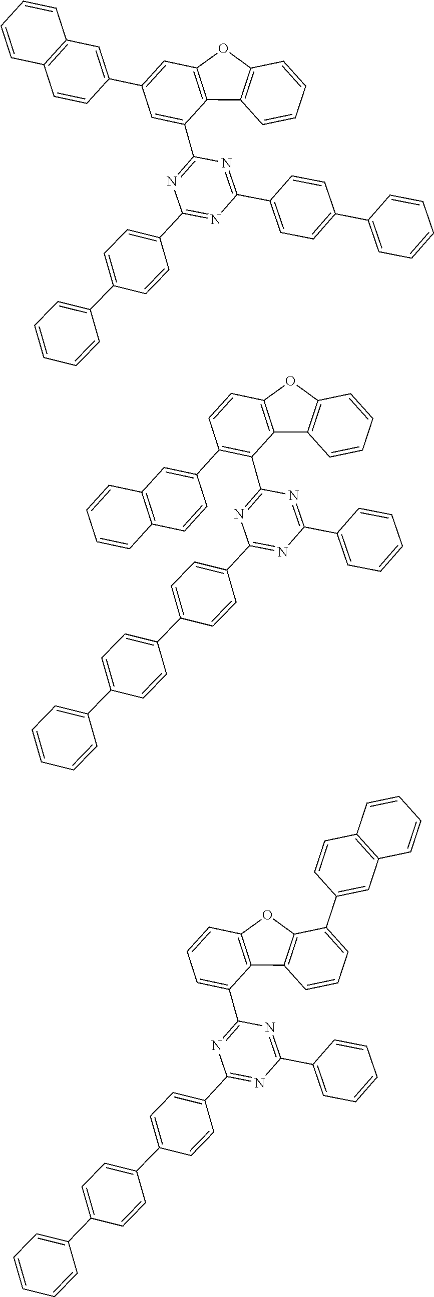 [SMILES: c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

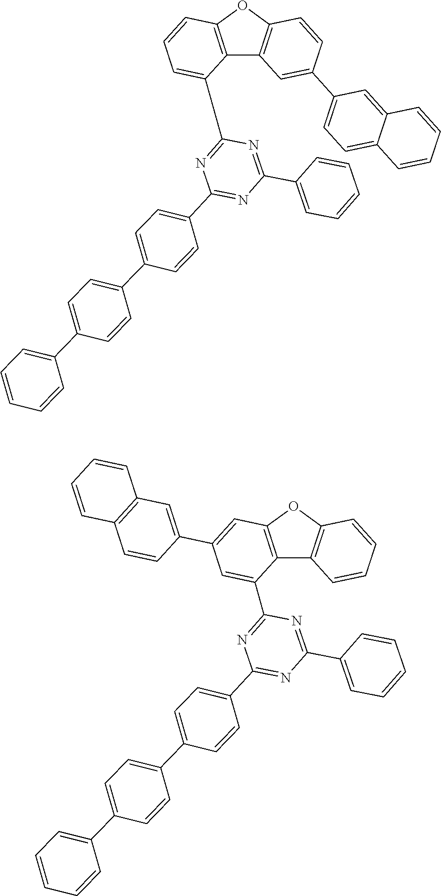 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

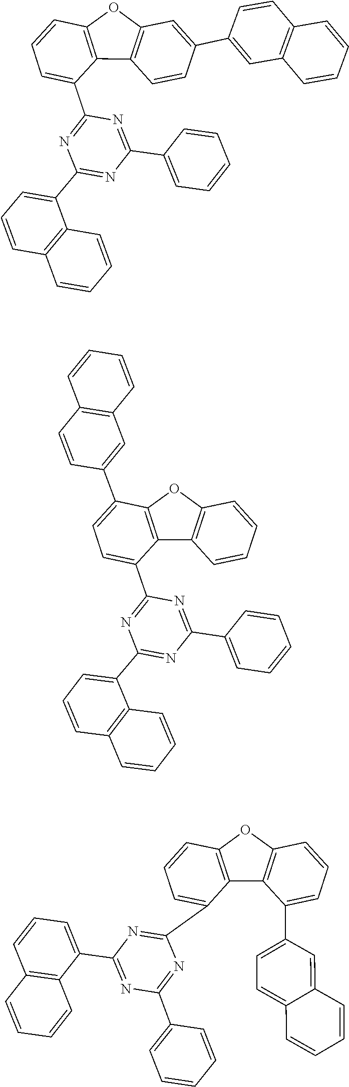 [SMILES: c1ccc(-c2nc(-c3cccc4ccccc34)nc(-c3cccc4oc5cc(-c6ccc7ccccc7c6)ccc5c34)n2)cc1 ; c1ccc(-c2nc(-c3cccc4ccccc34)nc(-c3ccc(-c4ccc5ccccc5c4)c4oc5ccccc5c34)n2)cc1 ; c1ccc(-c2nc(-c3cccc4ccccc34)nc(-c3cccc4oc5cccc(-c6ccc7ccccc7c6)c5c34)n2)cc1]

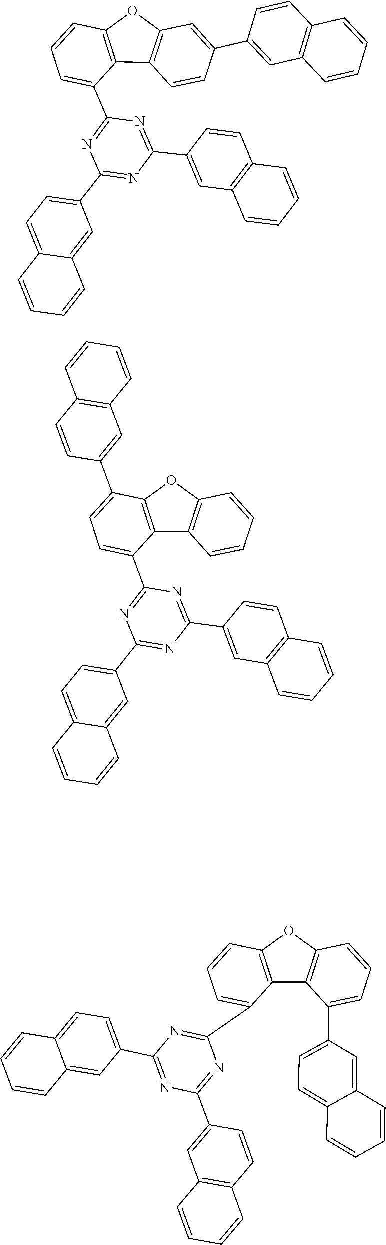

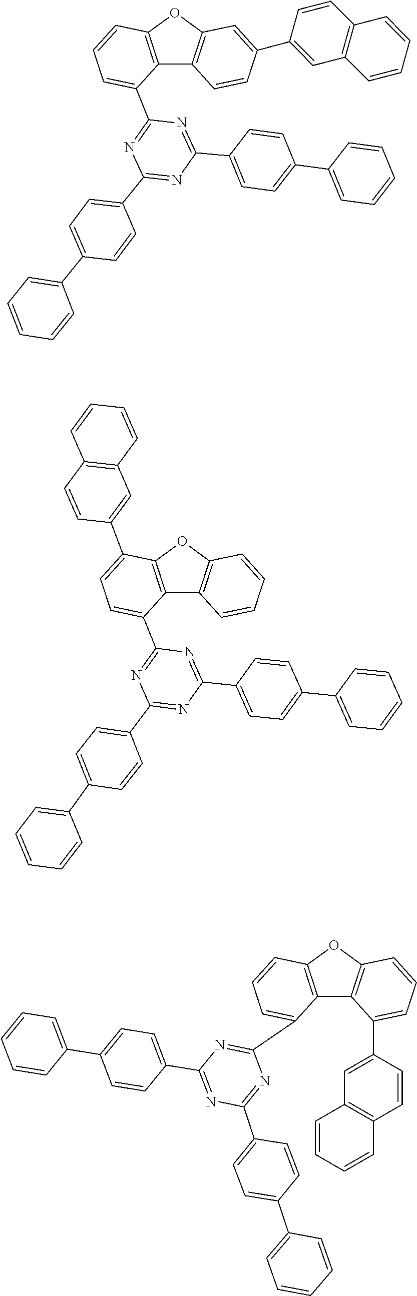 [SMILES: c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1]

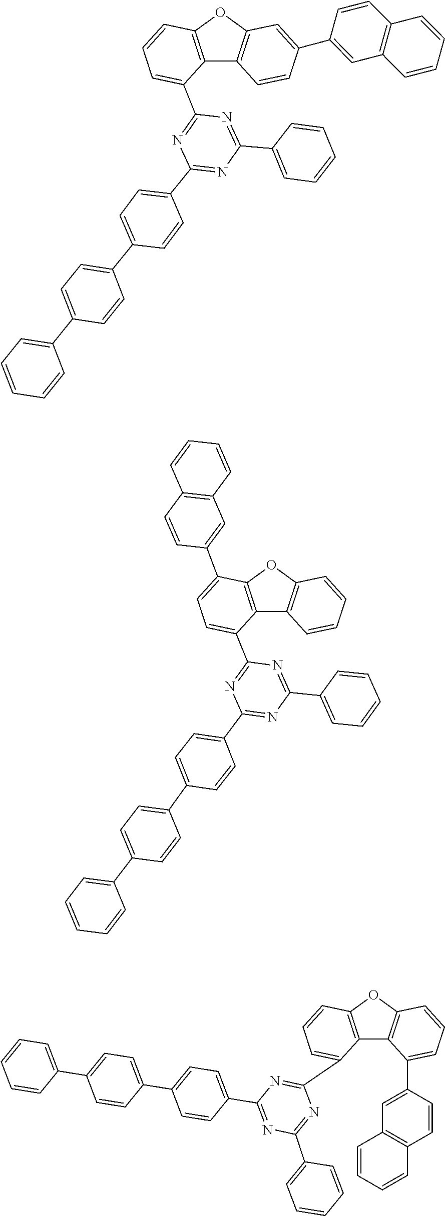 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

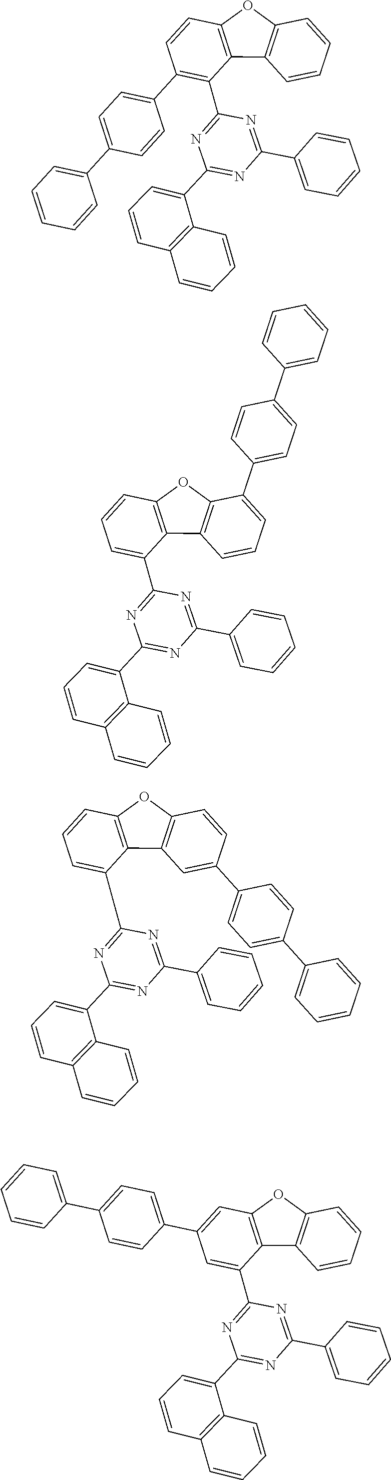

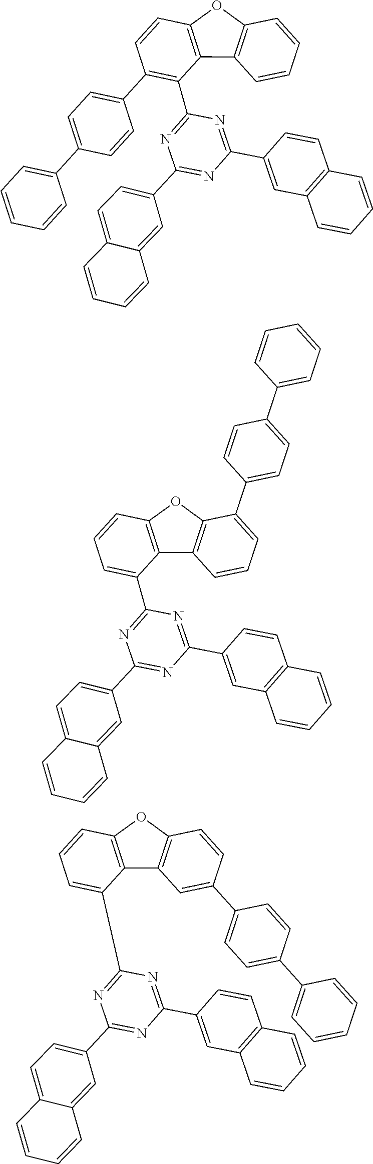

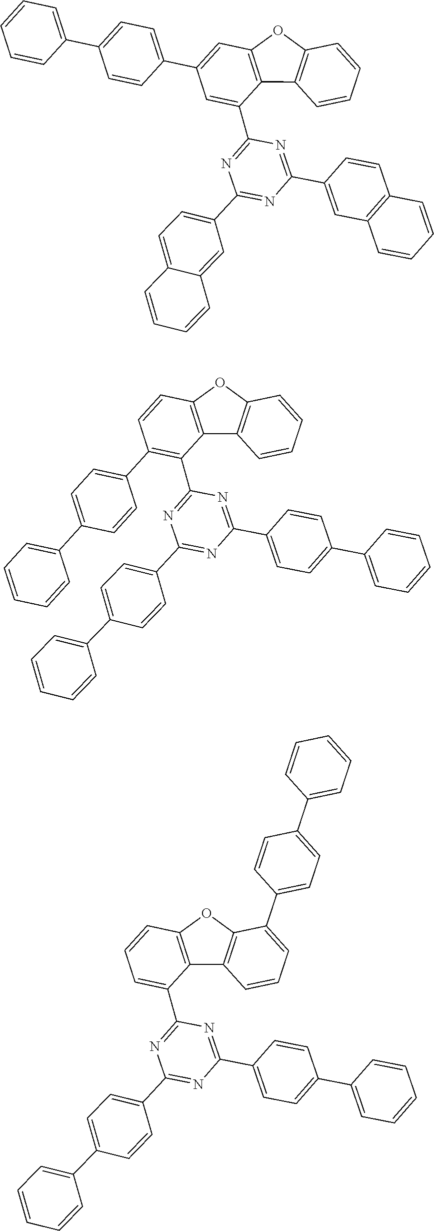 [SMILES: c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1]

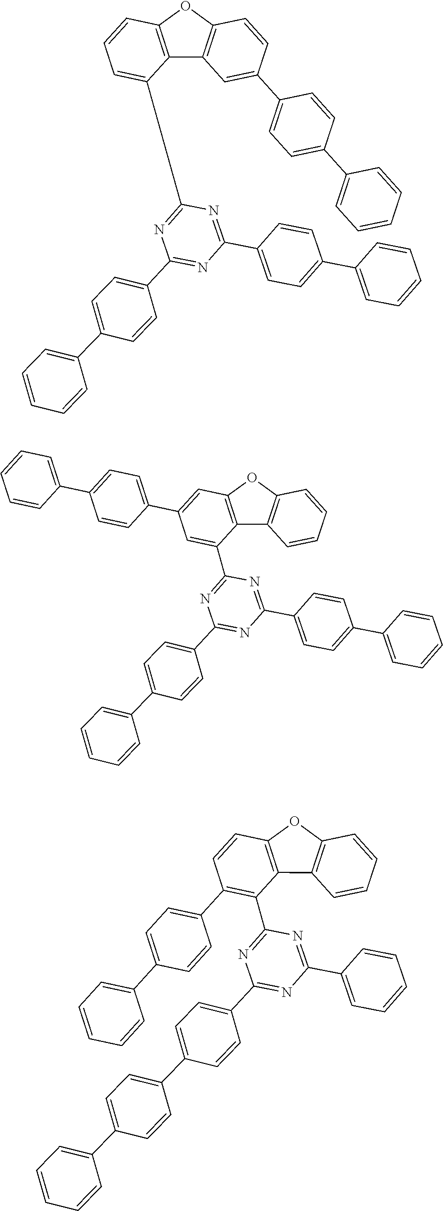

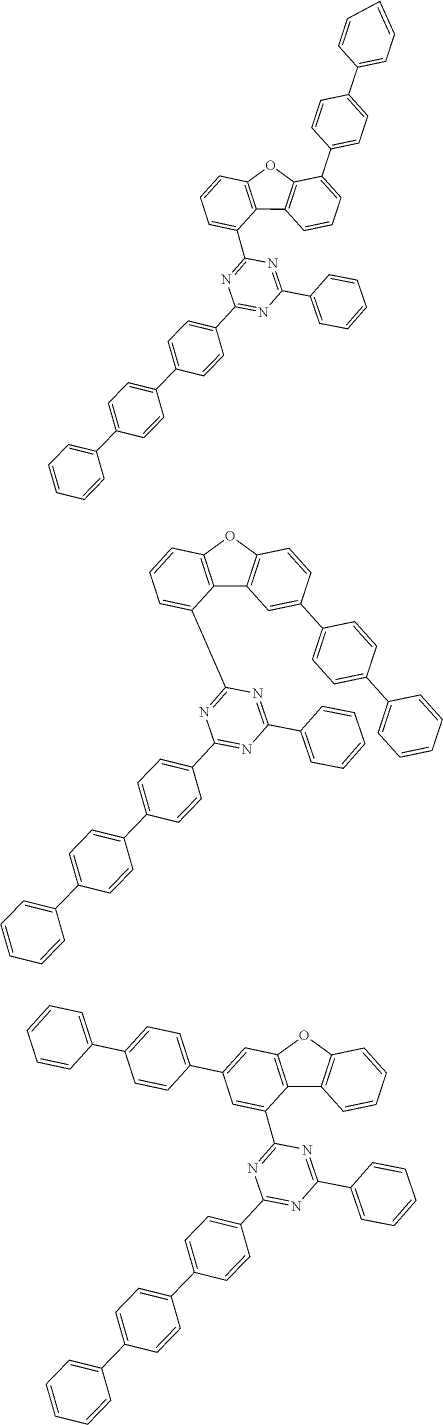

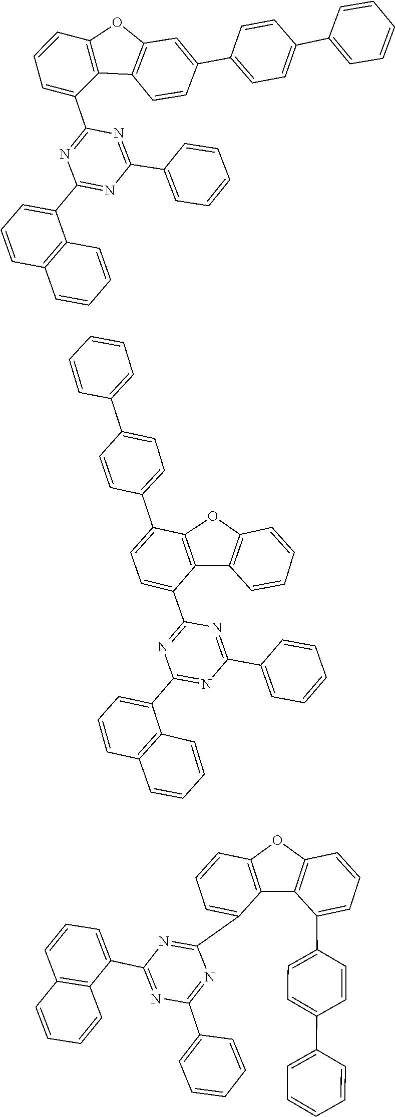

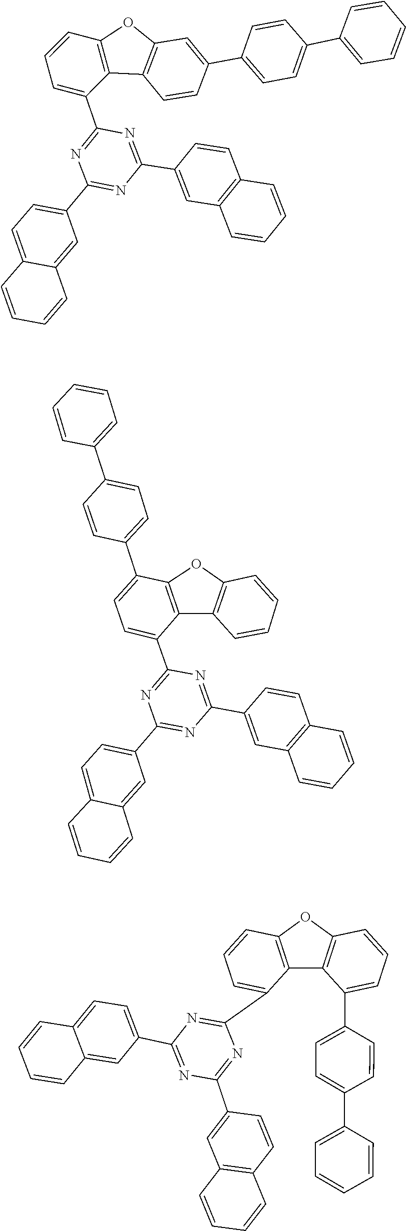 [SMILES: c1ccc(-c2ccc(-c3ccc4c(c3)oc3cccc(-c5nc(-c6ccc7ccccc7c6)nc(-c6ccc7ccccc7c6)n5)c34)cc2)cc1 ; c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1]

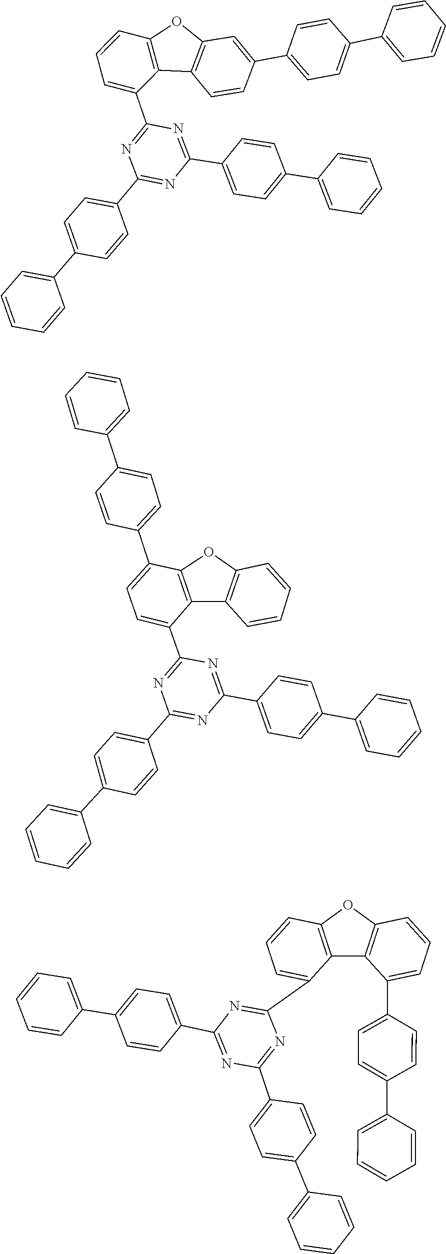 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1]

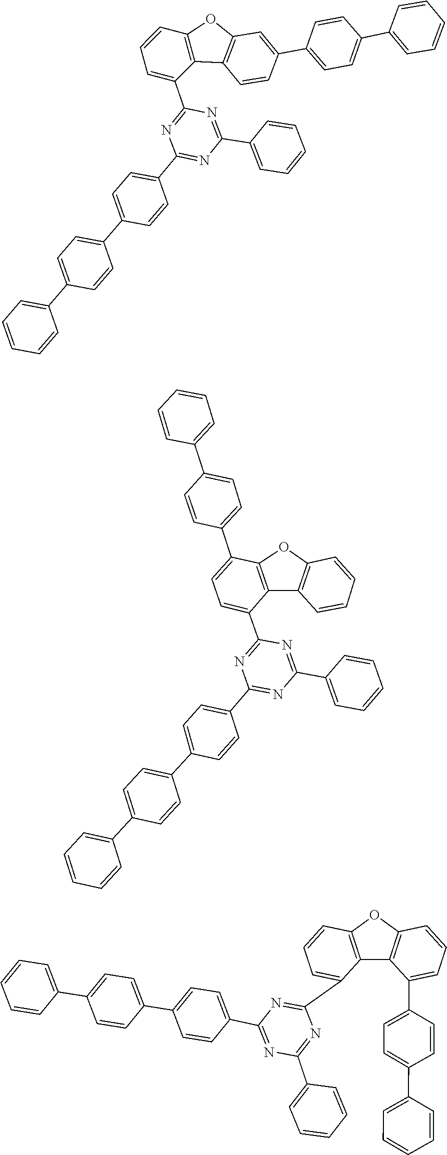 [SMILES: c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

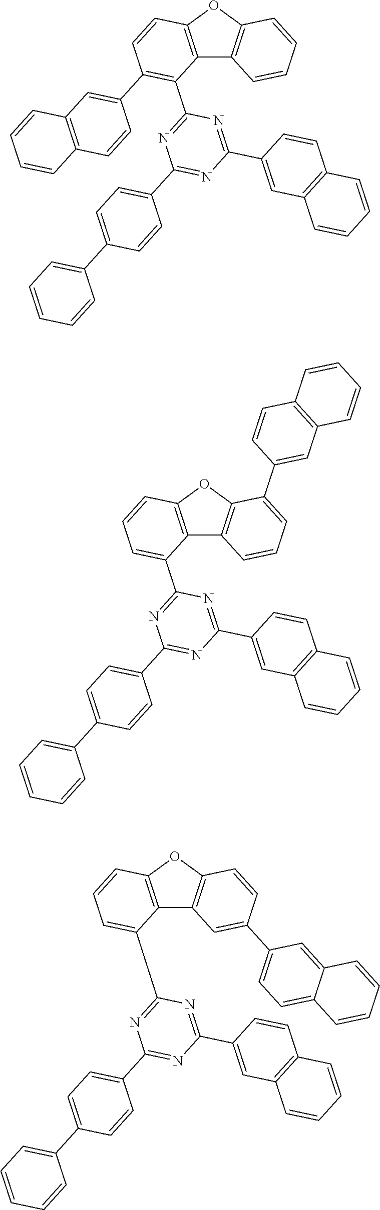 [SMILES: c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1 ; c1ccc(-c2ccc(-c3nc(-c4ccc5ccccc5c4)nc(-c4cccc5oc6ccc(-c7ccc8ccccc8c7)cc6c45)n3)cc2)cc1]

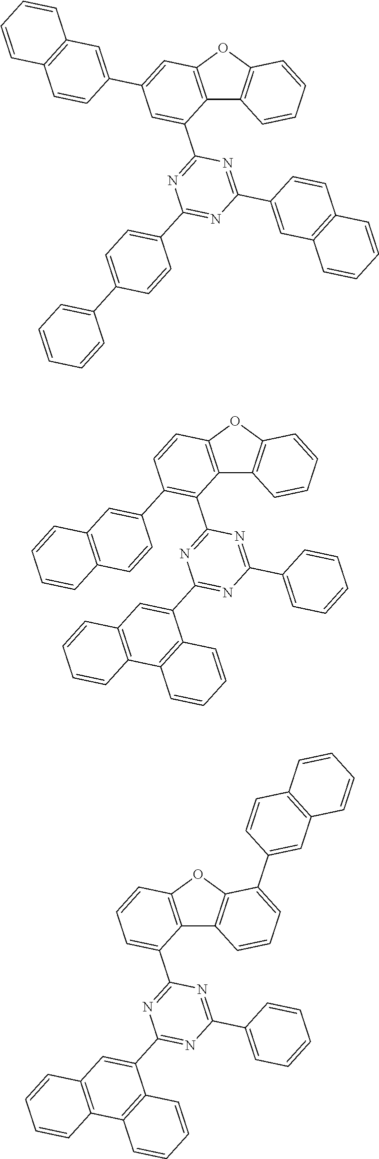 [SMILES: c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2nc(-c3cc4ccccc4c4ccccc34)nc(-c3c(-c4ccc5ccccc5c4)ccc4oc5ccccc5c34)n2)cc1 ; c1ccc(-c2nc(-c3cc4ccccc4c4ccccc34)nc(-c3cccc4oc5c(-c6ccc7ccccc7c6)cccc5c34)n2)cc1]

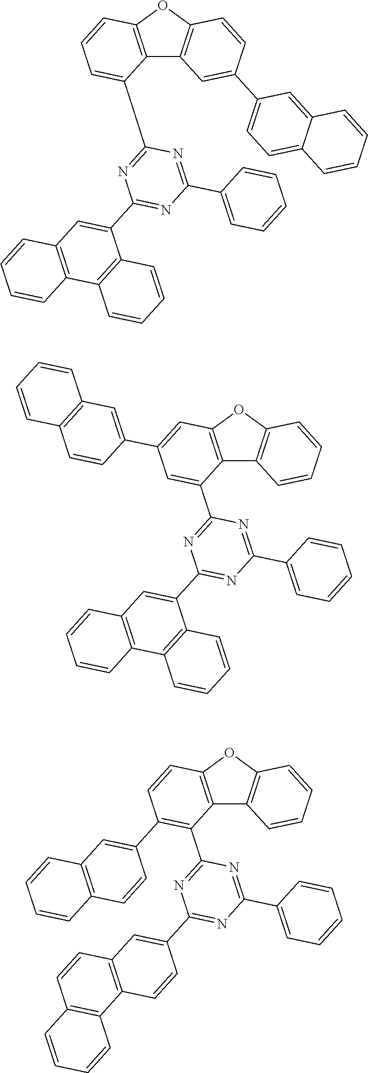 [SMILES: c1ccc(-c2nc(-c3cc4ccccc4c4ccccc34)nc(-c3cccc4oc5ccc(-c6ccc7ccccc7c6)cc5c34)n2)cc1 ; c1ccc(-c2nc(-c3cc4ccccc4c4ccccc34)nc(-c3cc(-c4ccc5ccccc5c4)cc4oc5ccccc5c34)n2)cc1 ; c1ccc(-c2nc(-c3ccc4c(ccc5ccccc54)c3)nc(-c3c(-c4ccc5ccccc5c4)ccc4oc5ccccc5c34)n2)cc1]

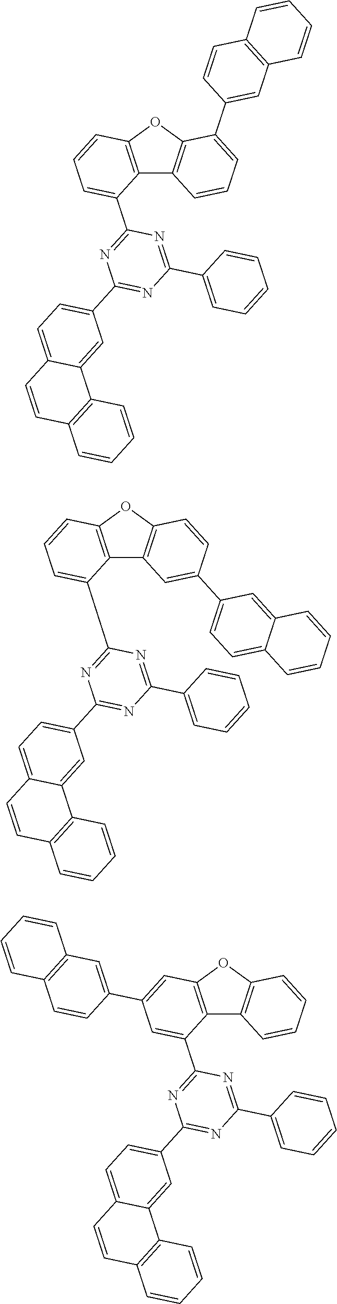

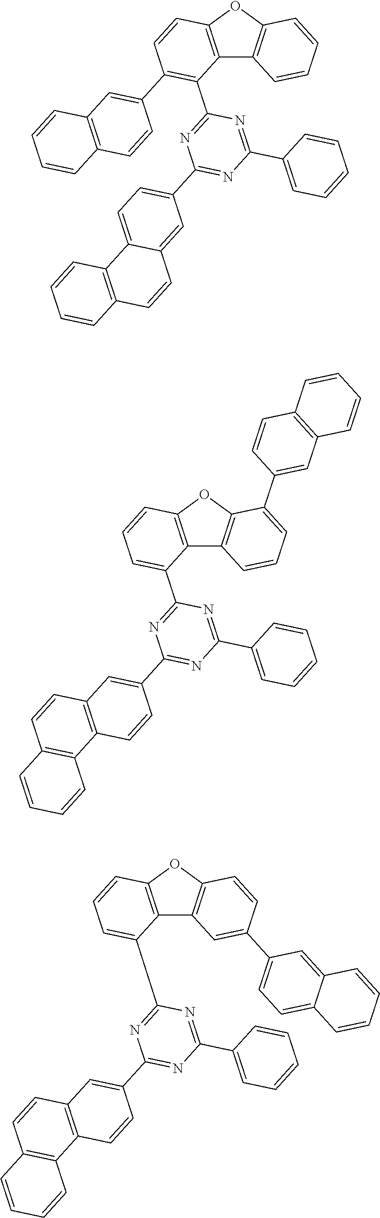 [SMILES: c1ccc(-c2nc(-c3ccc4c(ccc5ccccc54)c3)nc(-c3c(-c4ccc5ccccc5c4)ccc4oc5ccccc5c34)n2)cc1 ; c1ccc(-c2nc(-c3ccc4c(ccc5ccccc54)c3)nc(-c3cccc4oc5c(-c6ccc7ccccc7c6)cccc5c34)n2)cc1 ; c1ccc(-c2nc(-c3ccc4c(ccc5ccccc54)c3)nc(-c3cccc4oc5ccc(-c6ccc7ccccc7c6)cc5c34)n2)cc1]

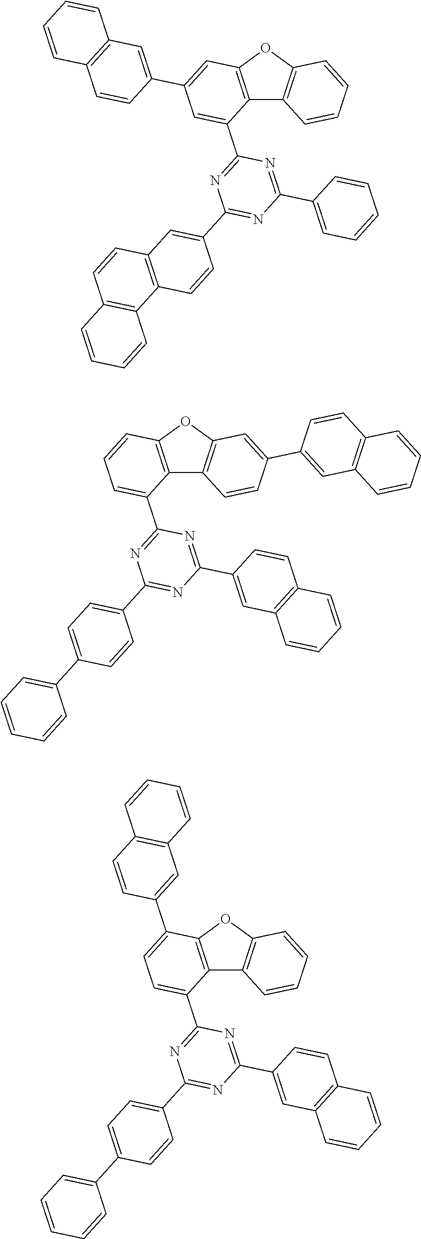 [SMILES: c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1]

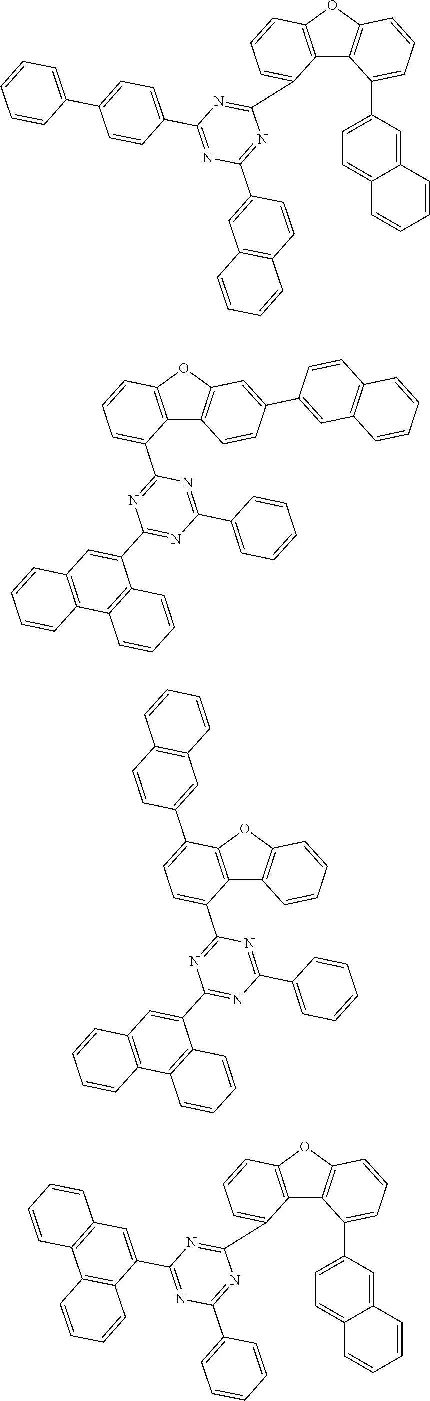

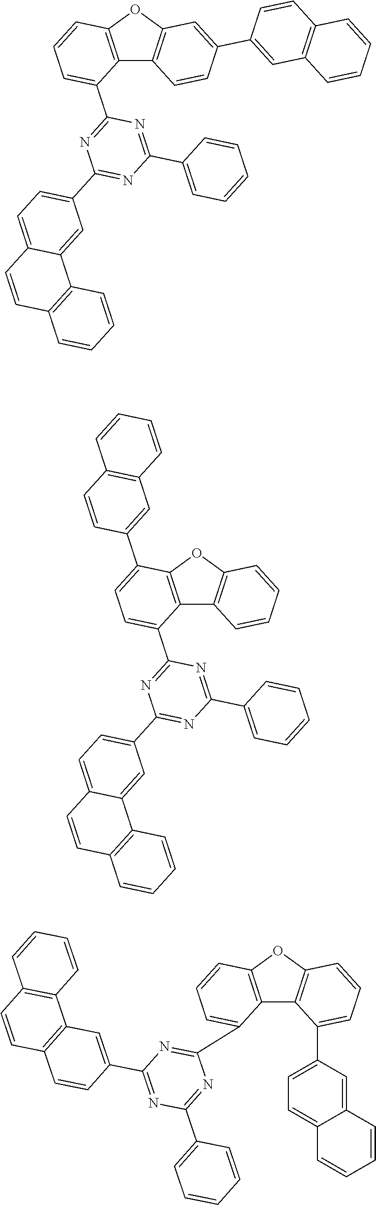 [SMILES: c1ccc(-c2nc(-c3ccc4ccc5ccccc5c4c3)nc(-c3cccc4oc5cc(-c6ccc7ccccc7c6)ccc5c34)n2)cc1 ; c1ccc(-c2nc(-c3ccc4ccc5ccccc5c4c3)nc(-c3ccc(-c4ccc5ccccc5c4)c4oc5ccccc5c34)n2)cc1 ; c1ccc(-c2nc(-c3ccc4ccc5ccccc5c4c3)nc(-c3cccc4oc5cccc(-c6ccc7ccccc7c6)c5c34)n2)cc1]

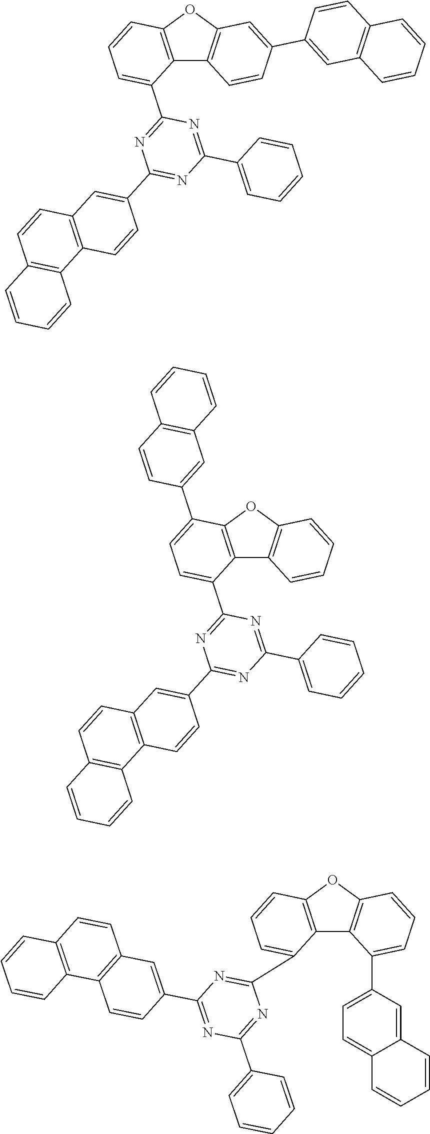 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1 ; c1ccc(-c2nc(-c3ccc4c(ccc5ccccc54)c3)nc(-c3cccc4oc5cccc(-c6ccc7ccccc7c6)c5c34)n2)cc1]

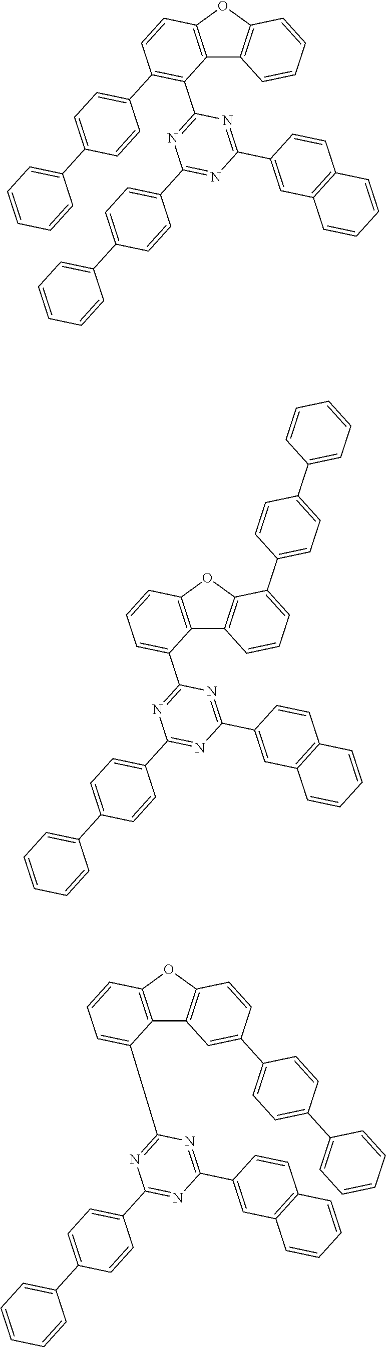 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1]

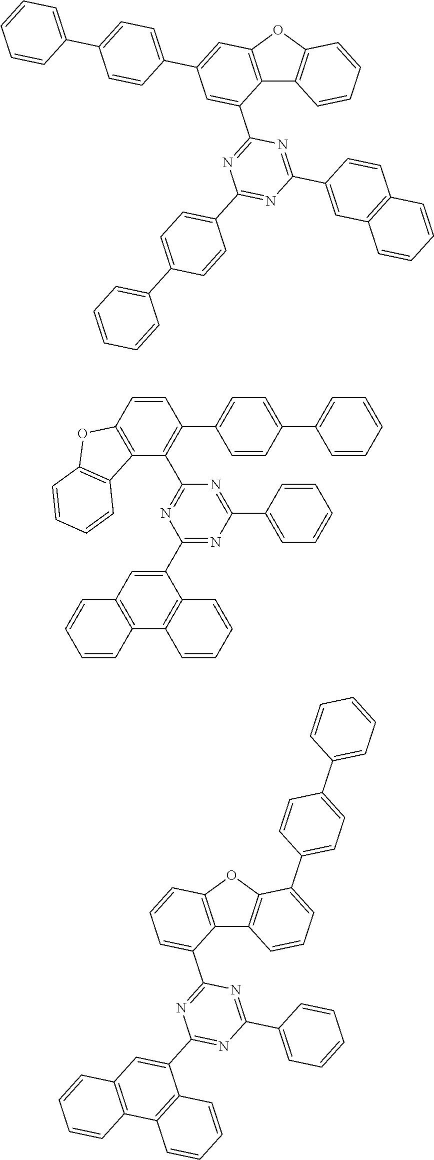 [SMILES: c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4oc5ccccc5c4c3-c3nc(-c4ccccc4)nc(-c4cc5ccccc5c5ccccc45)n3)cc2)cc1 ; c1ccc(-c2ccc(-c3cccc4c3oc3cccc(-c5nc(-c6ccccc6)nc(-c6cc7ccccc7c7ccccc67)n5)c34)cc2)cc1]

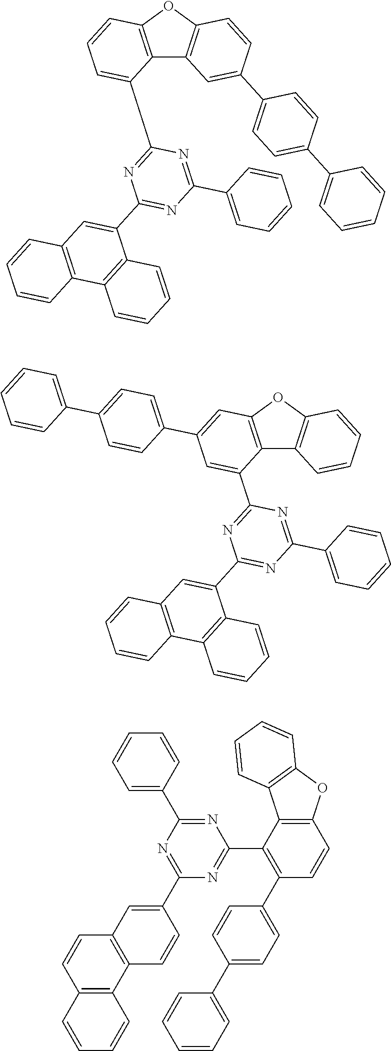

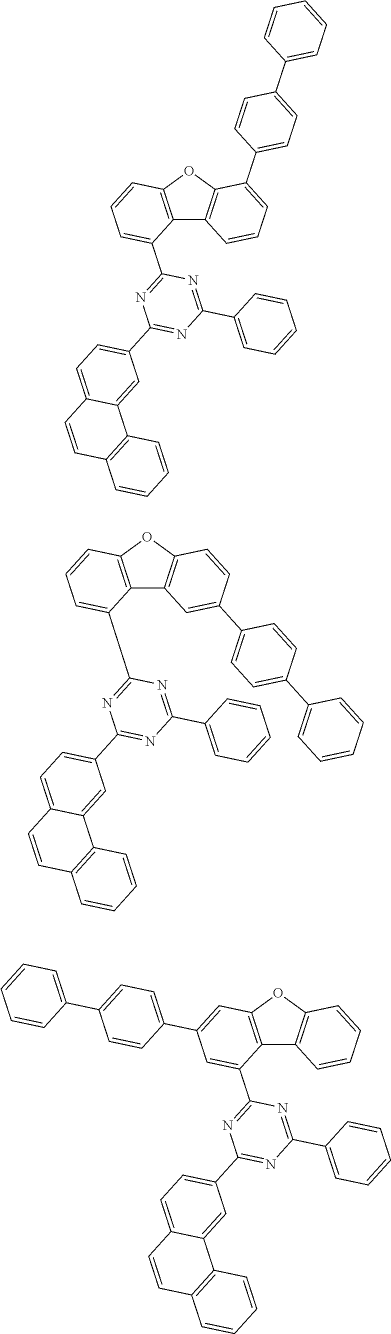

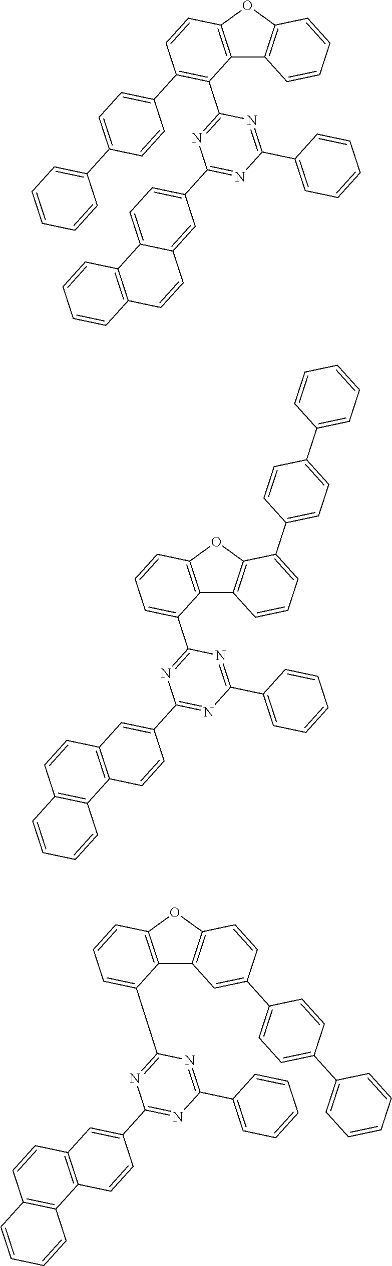 [SMILES: c1ccc(-c2ccc(-c3ccc4oc5ccccc5c4c3-c3nc(-c4ccccc4)nc(-c4ccc5c(ccc6ccccc65)c4)n3)cc2)cc1 ; c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1]

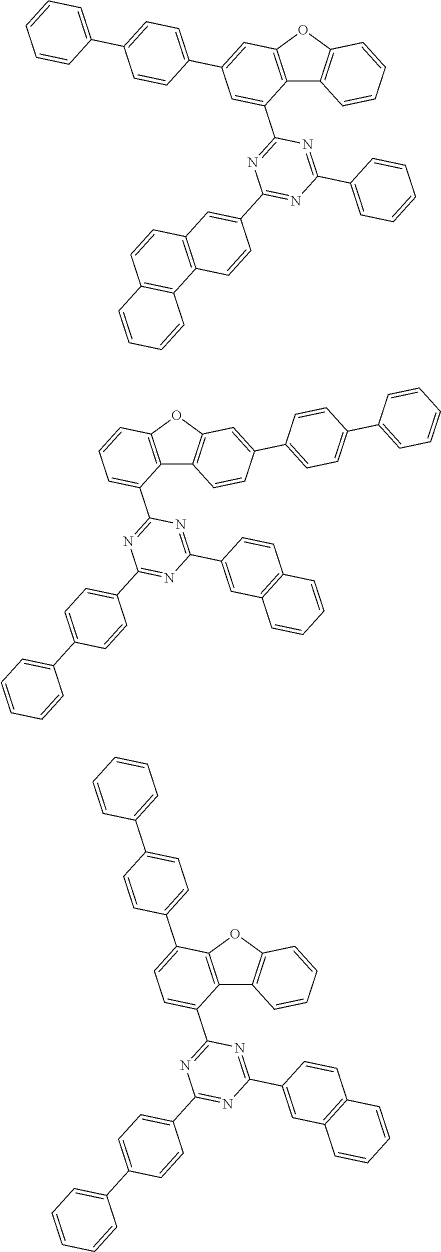 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1 ; c1ccc(-c2ccc(-c3nc(-c4ccc5ccccc5c4)nc(-c4ccc(-c5ccc(-c6ccccc6)cc5)c5oc6ccccc6c45)n3)cc2)cc1]

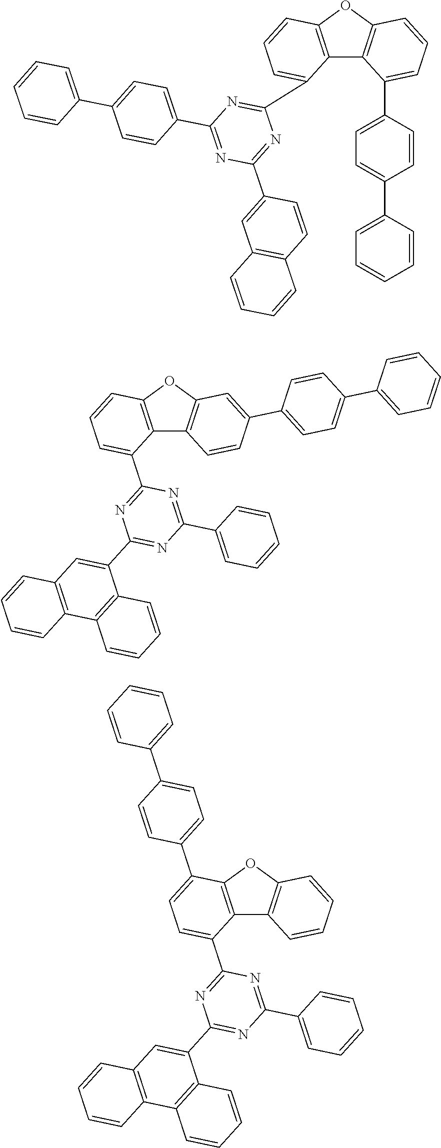

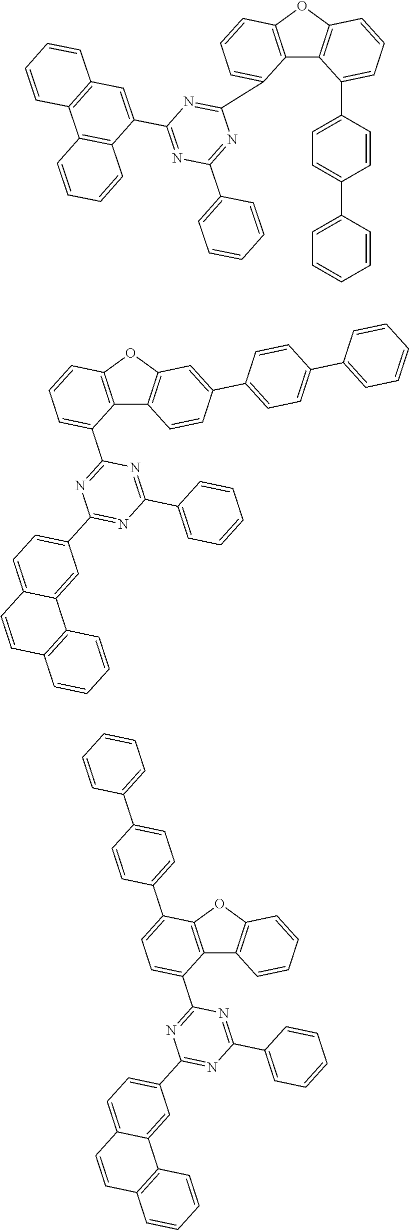

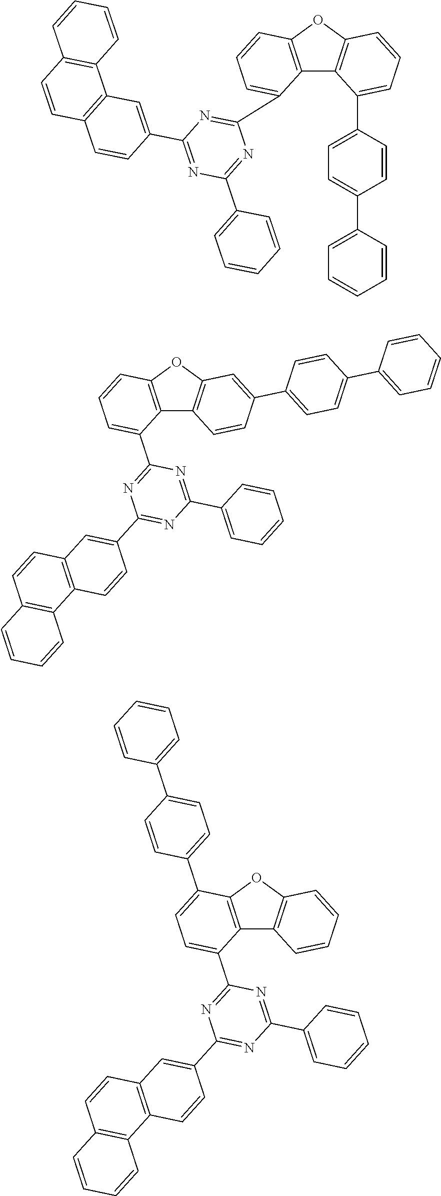 [SMILES: c1ccc(-c2ccc(-c3cccc4oc5cccc(-c6nc(-c7ccccc7)nc(-c7ccc8ccc9ccccc9c8c7)n6)c5c34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1]

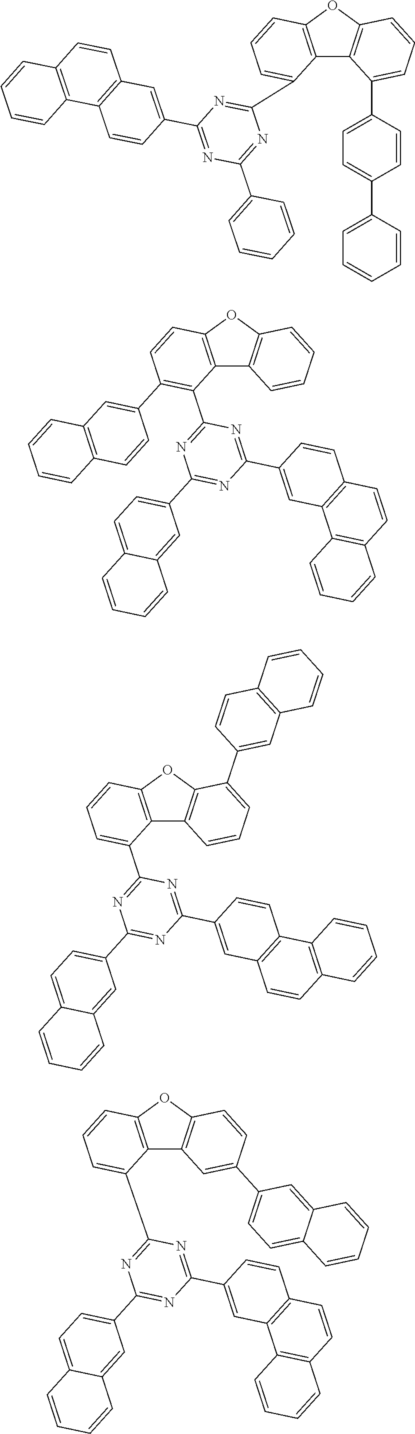

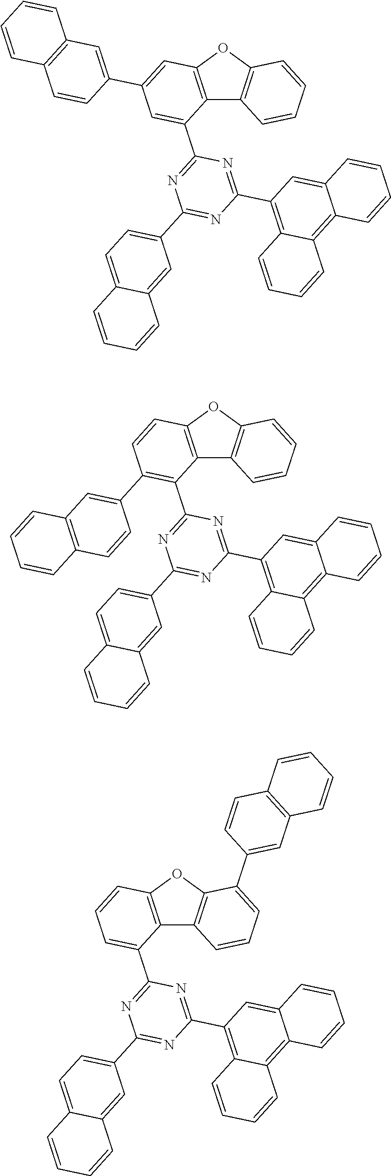 [SMILES: c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3nc(-c4cc5ccccc5c5ccccc45)nc(-c4c(-c5ccc6ccccc6c5)ccc5oc6ccccc6c45)n3)ccc2c1 ; c1ccc2cc(-c3nc(-c4cc5ccccc5c5ccccc45)nc(-c4cccc5oc6c(-c7ccc8ccccc8c7)cccc6c45)n3)ccc2c1]

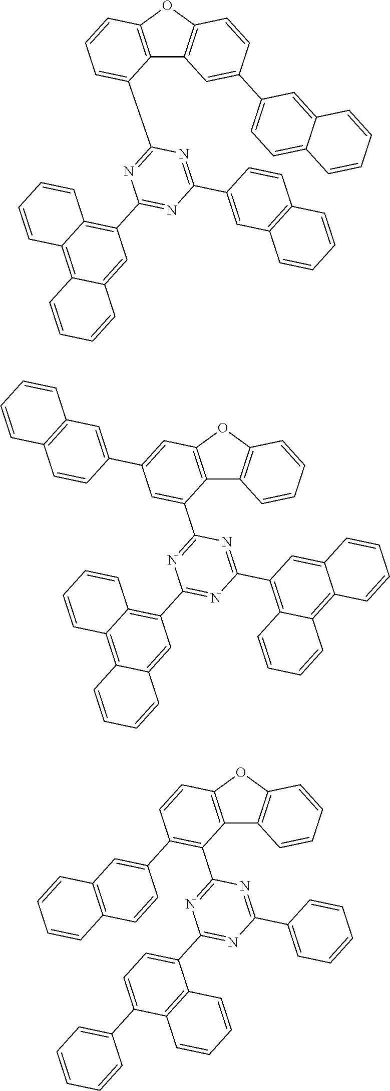 [SMILES: c1ccc2cc(-c3ccc4oc5cccc(-c6nc(-c7ccc8ccccc8c7)nc(-c7cc8ccccc8c8ccccc78)n6)c5c4c3)ccc2c1 ; c1ccc2cc(-c3cc(-c4nc(-c5cc6ccccc6c6ccccc56)nc(-c5cc6ccccc6c6ccccc56)n4)c4c(c3)oc3ccccc34)ccc2c1 ; c1ccc(-c2nc(-c3ccc(-c4ccccc4)c4ccccc34)nc(-c3c(-c4ccc5ccccc5c4)ccc4oc5ccccc5c34)n2)cc1]

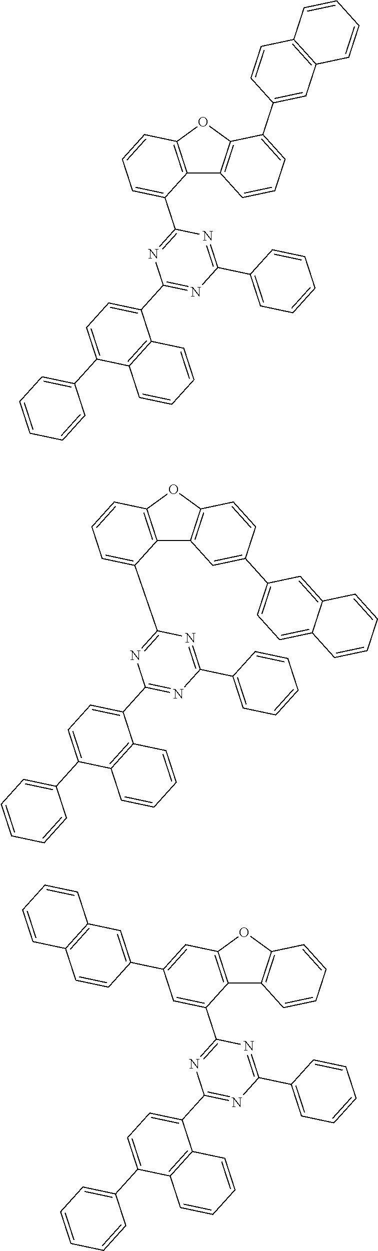

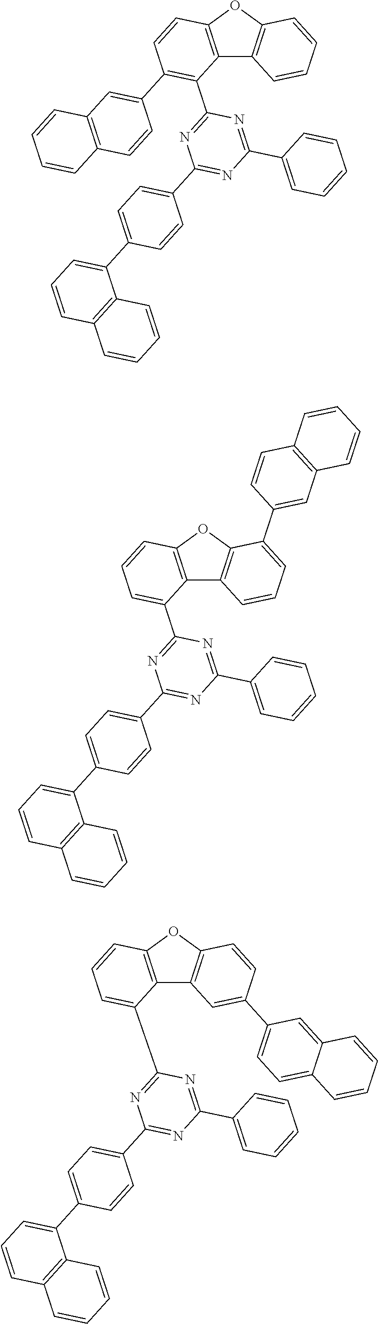 [SMILES: c1ccc(-c2nc(-c3ccc(-c4cccc5ccccc45)cc3)nc(-c3c(-c4ccc5ccccc5c4)ccc4oc5ccccc5c34)n2)cc1 ; c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1]

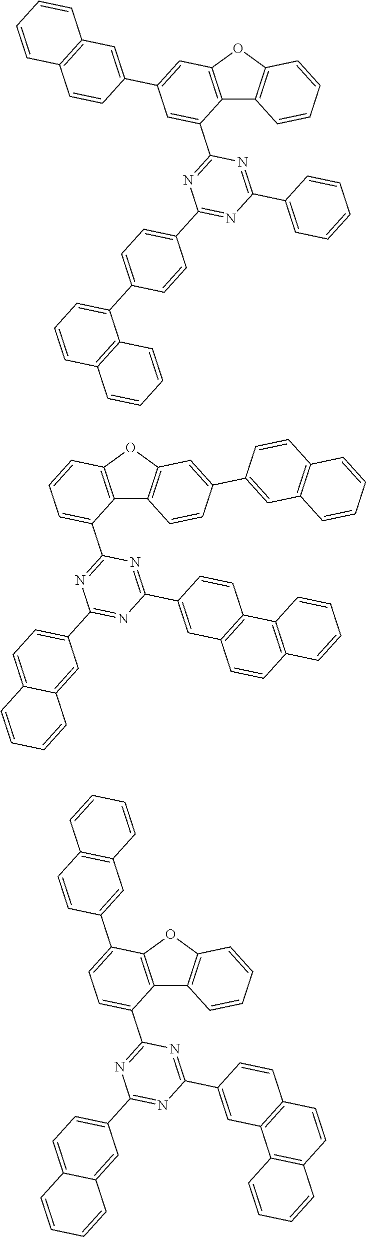

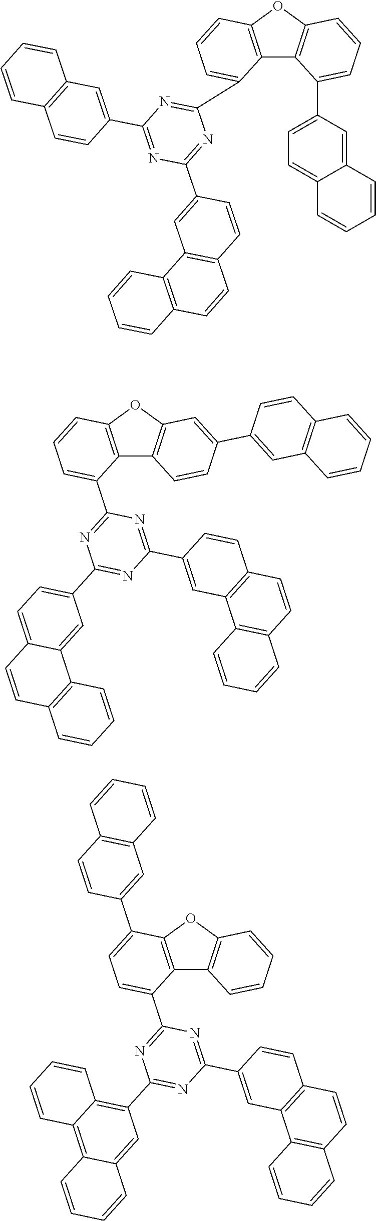

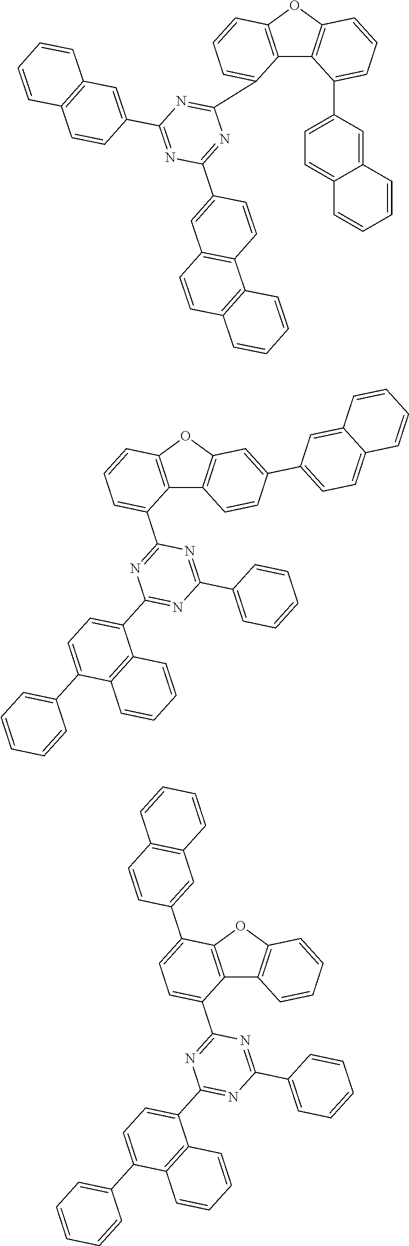 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1 ; c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1]

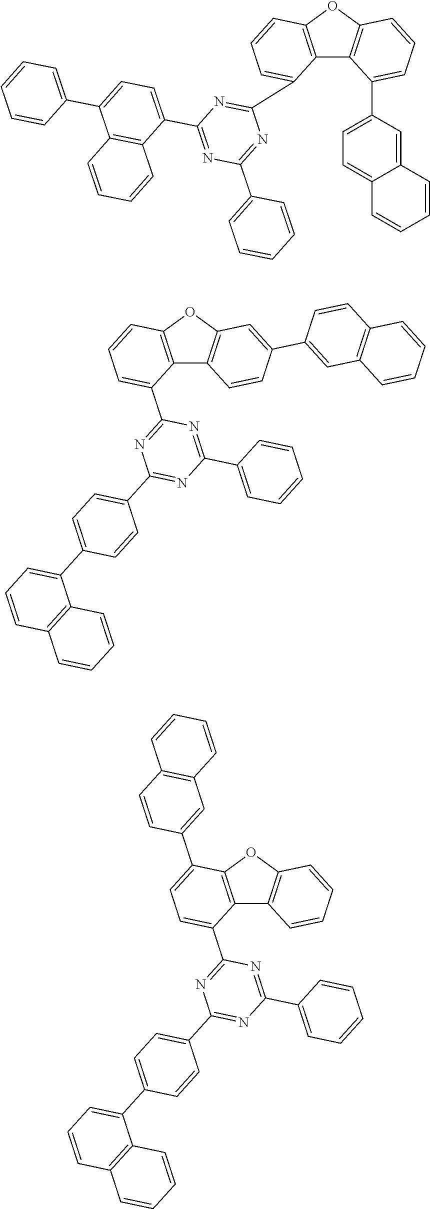 [SMILES: c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1]

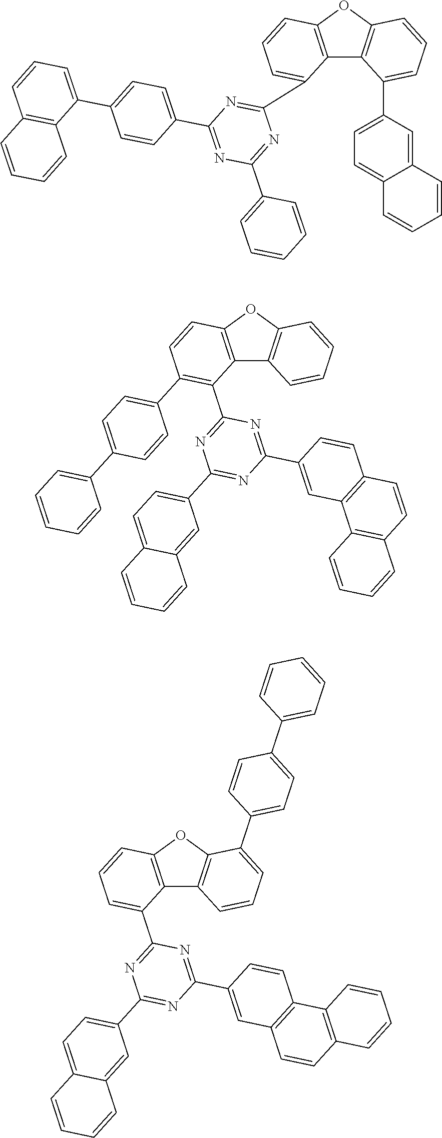 [SMILES: c1ccc(-c2nc(-c3ccc(-c4cccc5ccccc45)cc3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3cccc4c3oc3cccc(-c5nc(-c6ccc7ccccc7c6)nc(-c6ccc7c(ccc8ccccc87)c6)n5)c34)cc2)cc1]

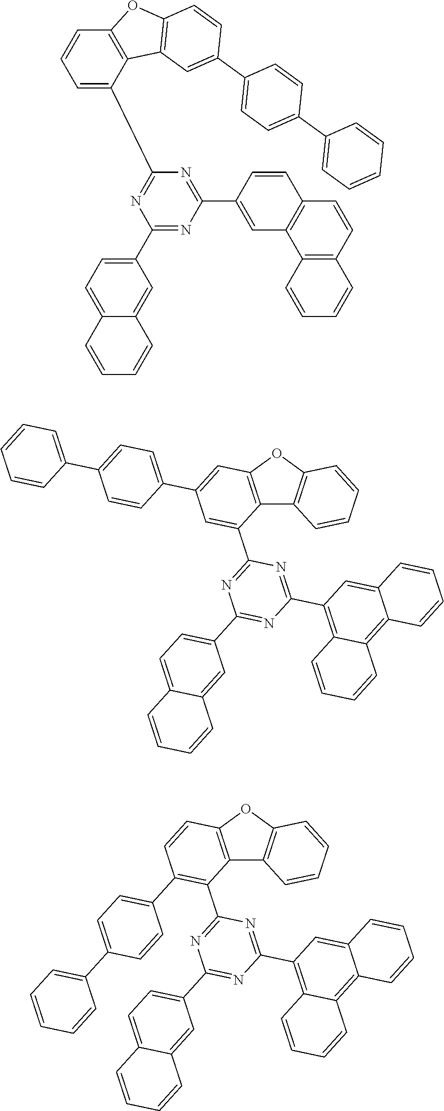 [SMILES: c1ccc(-c2ccc(-c3ccc4oc5cccc(-c6nc(-c7ccc8ccccc8c7)nc(-c7ccc8ccc9ccccc9c8c7)n6)c5c4c3)cc2)cc1 ; c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

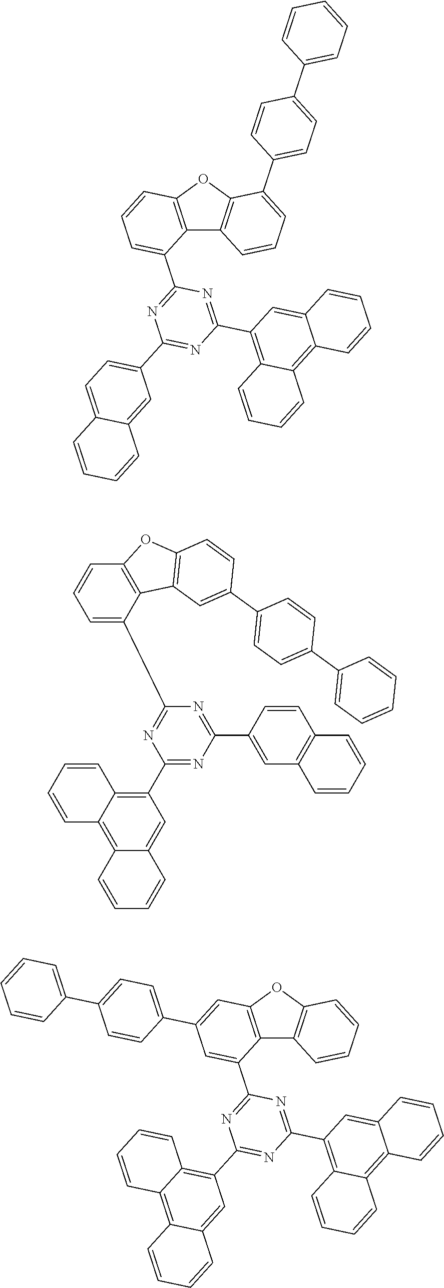 [SMILES: c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1 ; c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1]

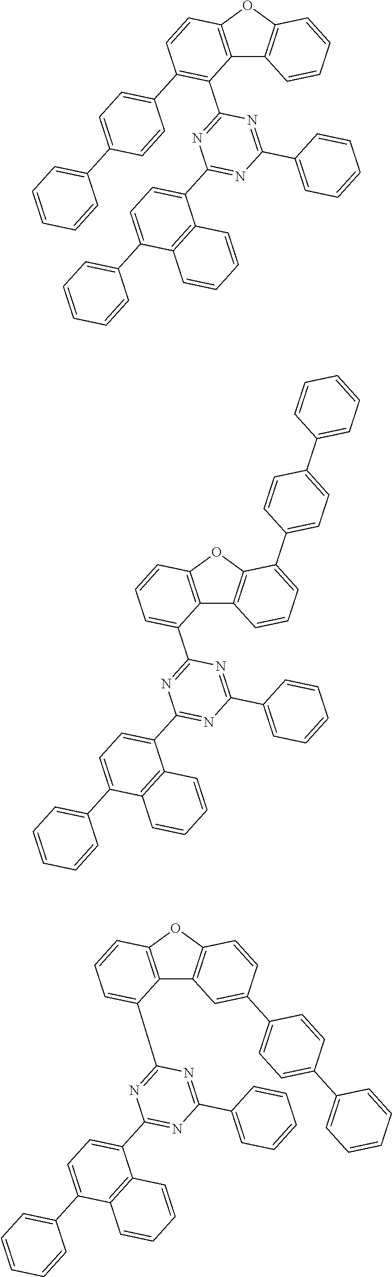 [SMILES: c1ccc(-c2ccc(-c3ccc4oc5ccccc5c4c3-c3nc(-c4ccccc4)nc(-c4ccc(-c5ccccc5)c5ccccc45)n3)cc2)cc1 ; c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1]

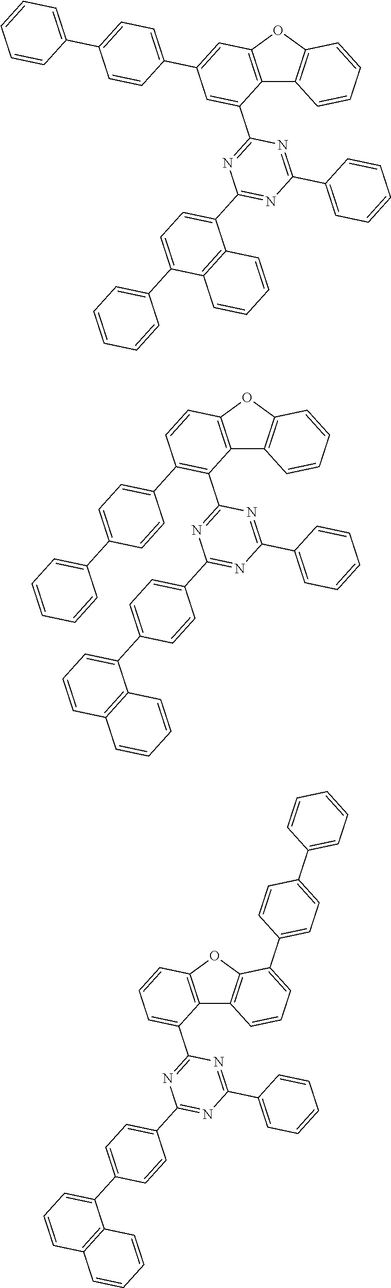 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1 ; c1ccc(-c2ccc(-c3cccc4c3oc3cccc(-c5nc(-c6ccccc6)nc(-c6ccc(-c7cccc8ccccc78)cc6)n5)c34)cc2)cc1]

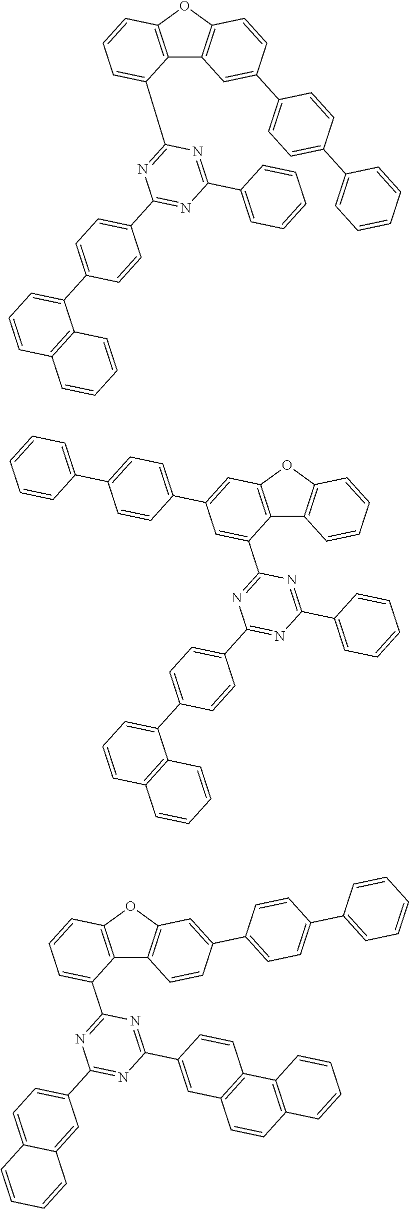 [SMILES: c1ccc(-c2ccc(-c3ccc4oc5cccc(-c6nc(-c7ccccc7)nc(-c7ccc(-c8cccc9ccccc89)cc7)n6)c5c4c3)cc2)cc1 ; c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1]

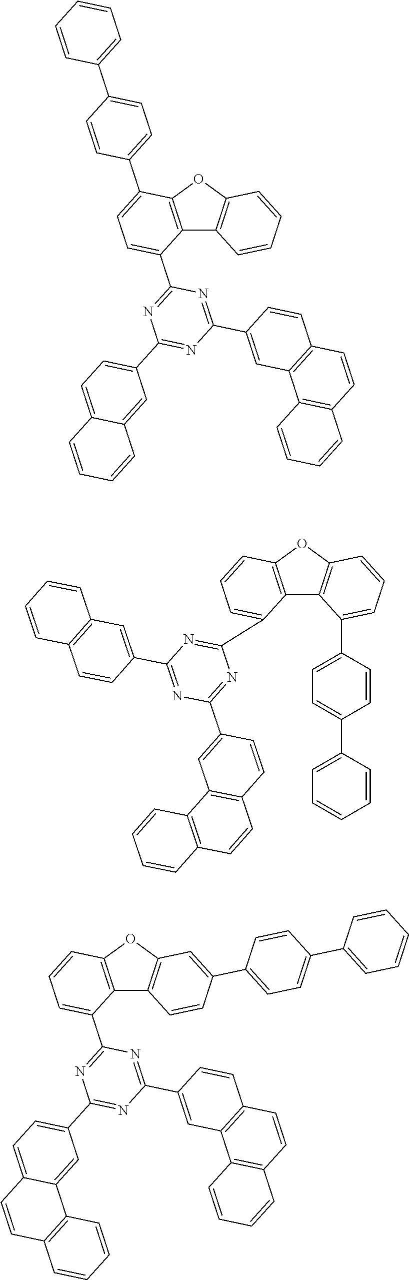 [SMILES: c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1 ; c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1]

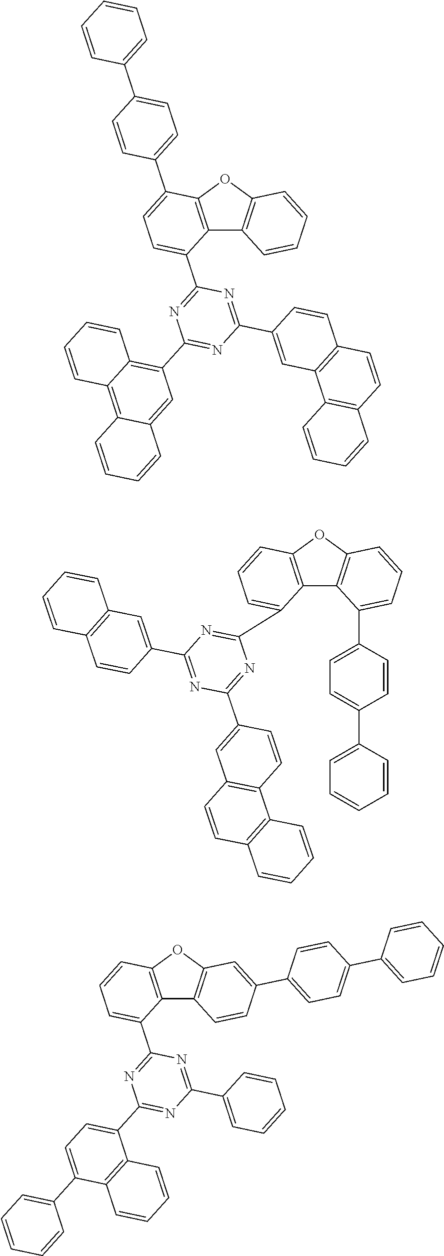 [SMILES: c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1 ; c1ccc(-c2ccc(-c3cccc4oc5cccc(-c6nc(-c7ccc8ccccc8c7)nc(-c7ccc8c(ccc9ccccc98)c7)n6)c5c34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1]

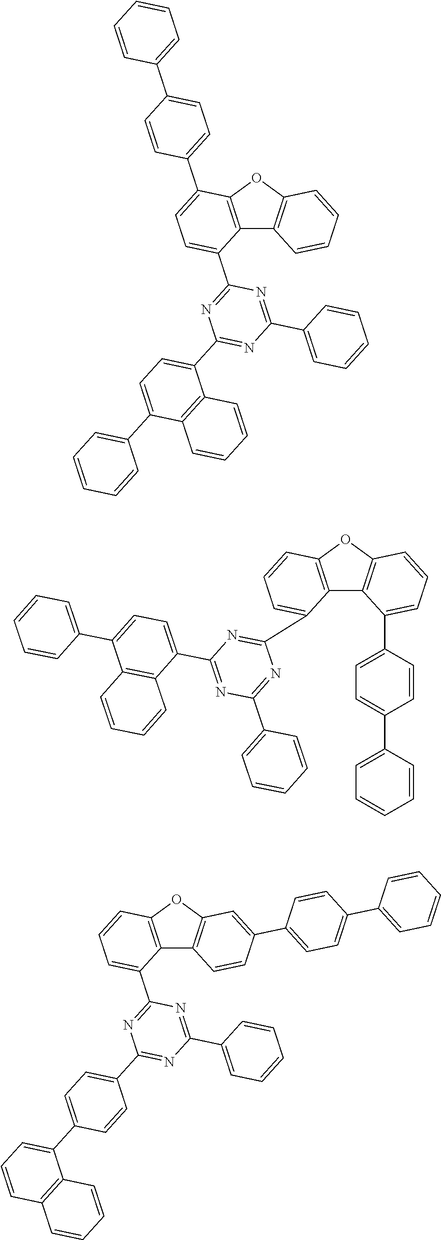 [SMILES: c1ccc(-c2ccc(-c3ccc(-c4nc(-c5ccccc5)nc(-c5ccc(-c6ccccc6)c6ccccc56)n4)c4c3oc3ccccc34)cc2)cc1 ; c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1]

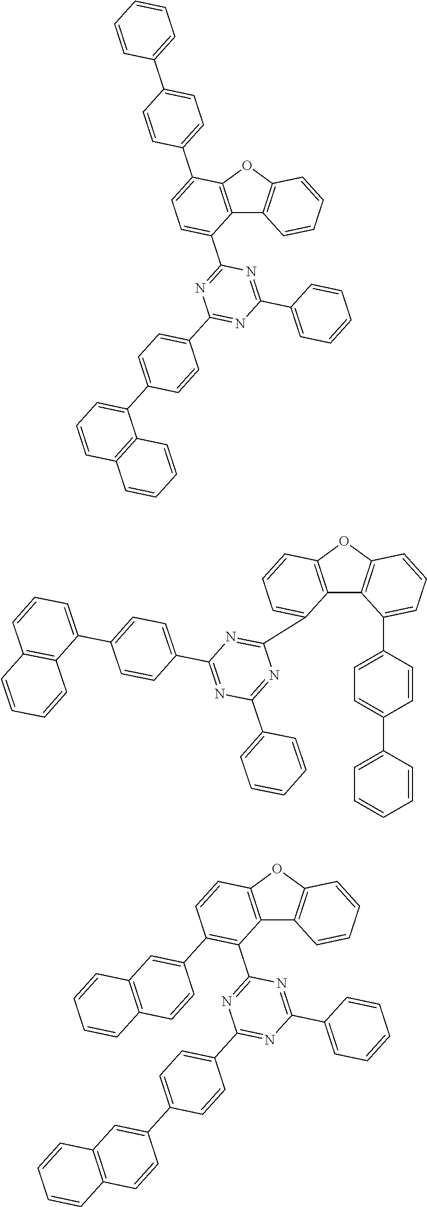 [SMILES: c1ccc(-c2ccc(-c3ccc(-c4nc(-c5ccccc5)nc(-c5ccc(-c6cccc7ccccc67)cc5)n4)c4c3oc3ccccc34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1 ; c1ccc(-c2nc(-c3ccc(-c4ccc5ccccc5c4)cc3)nc(-c3c(-c4ccc5ccccc5c4)ccc4oc5ccccc5c34)n2)cc1]

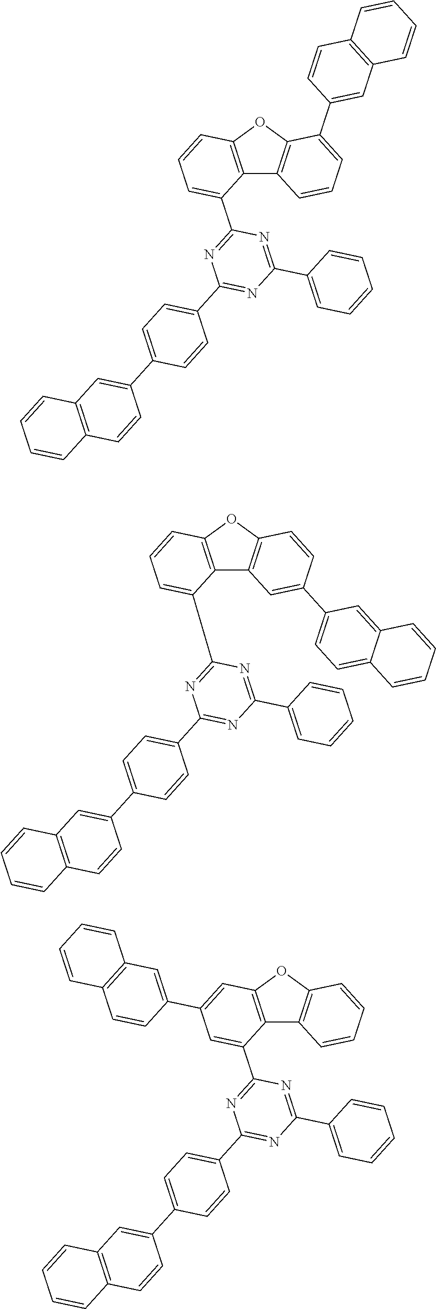 [SMILES: c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1]

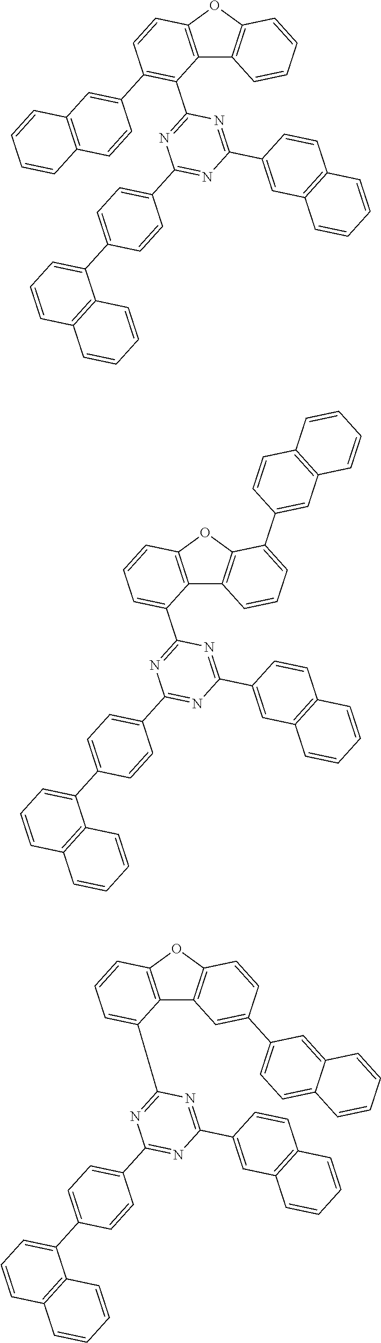 [SMILES: c1ccc2cc(-c3nc(-c4ccc(-c5cccc6ccccc56)cc4)nc(-c4c(-c5ccc6ccccc6c5)ccc5oc6ccccc6c45)n3)ccc2c1 ; c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1]

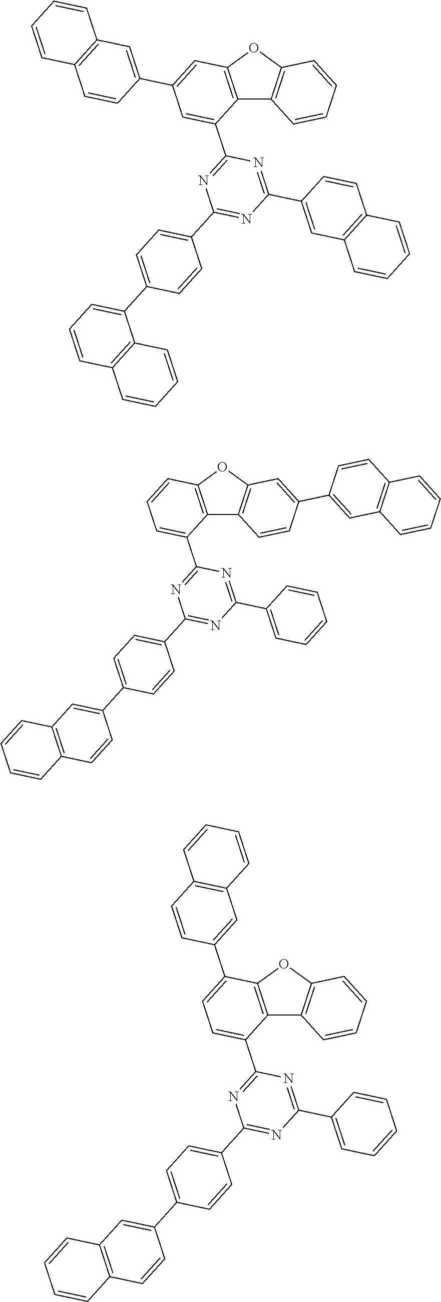 [SMILES: c1ccc2cc(-c3cc(-c4nc(-c5ccc(-c6cccc7ccccc67)cc5)nc(-c5ccc6ccccc6c5)n4)c4c(c3)oc3ccccc34)ccc2c1 ; c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

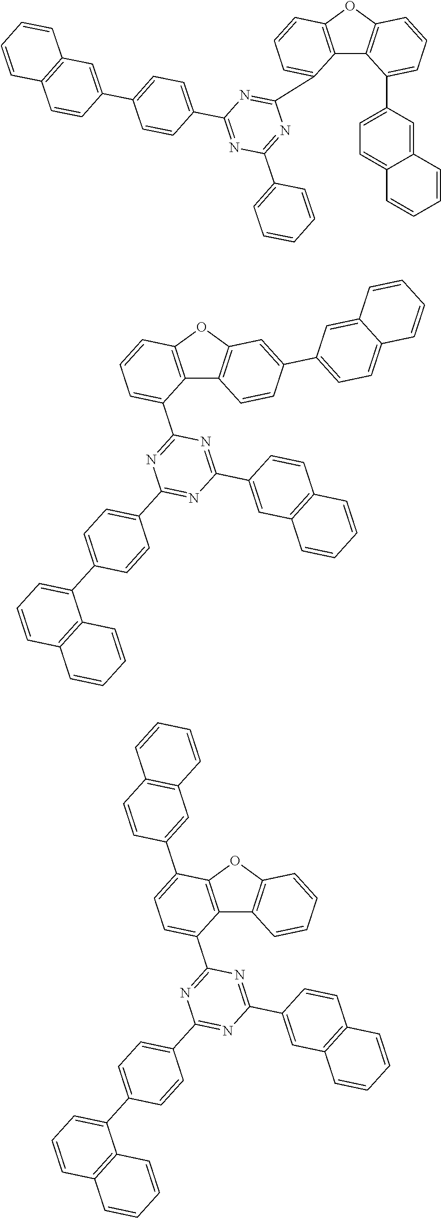 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1]

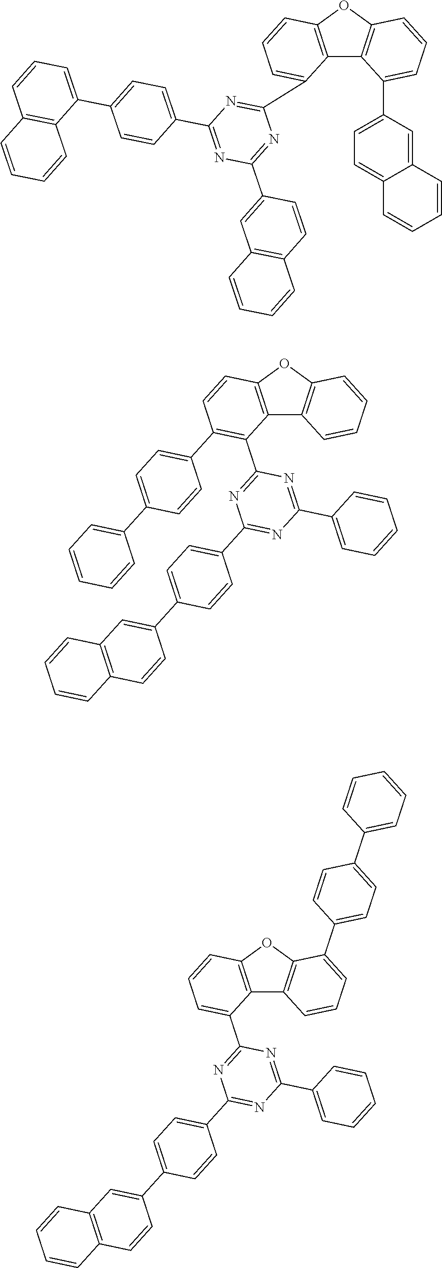 [SMILES: c1ccc2cc(-c3nc(-c4ccc(-c5cccc6ccccc56)cc4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1 ; c1ccc(-c2ccc(-c3cccc4c3oc3cccc(-c5nc(-c6ccccc6)nc(-c6ccc(-c7ccc8ccccc8c7)cc6)n5)c34)cc2)cc1]

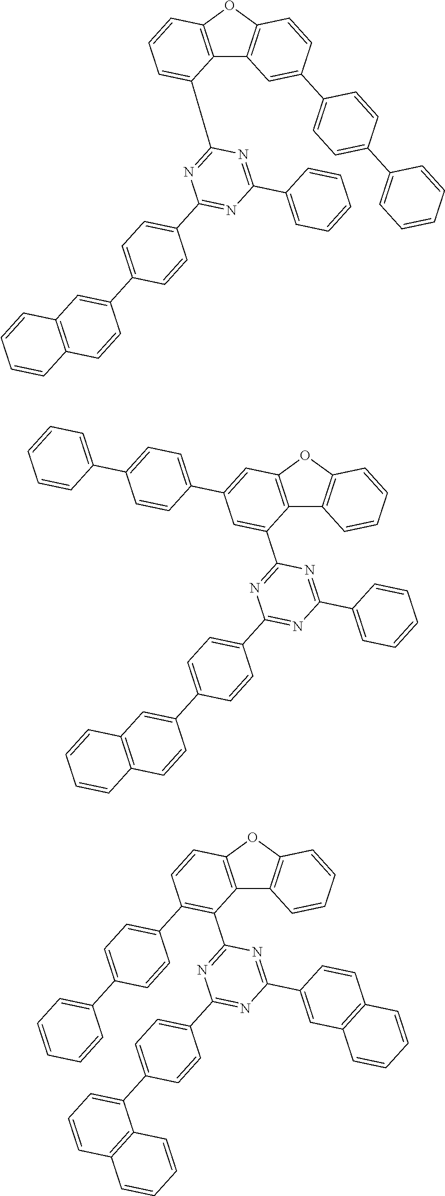 [SMILES: c1ccc(-c2ccc(-c3ccc4oc5cccc(-c6nc(-c7ccccc7)nc(-c7ccc(-c8ccc9ccccc9c8)cc7)n6)c5c4c3)cc2)cc1 ; c1ccc(-c2ccc(-c3cc(-c4nc(-c5ccccc5)nc(-c5ccc(-c6ccc7ccccc7c6)cc5)n4)c4c(c3)oc3ccccc34)cc2)cc1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

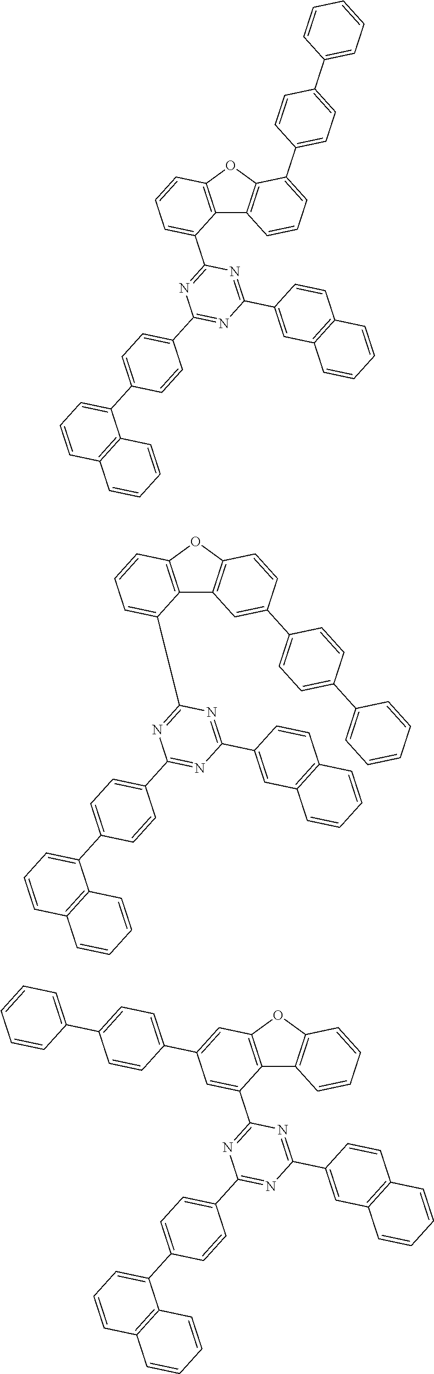 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1 ; c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1]

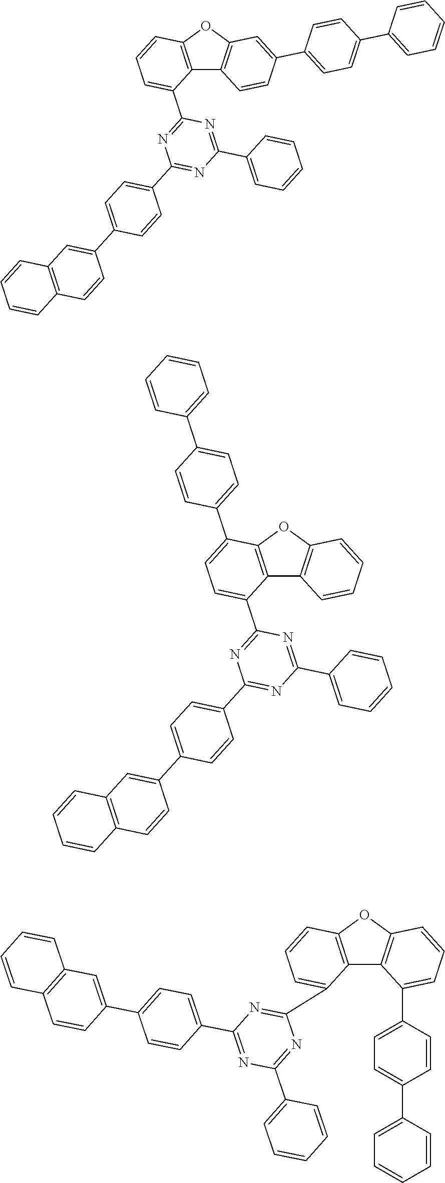 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1 ; c1ccc(-c2ccc(-c3cccc4oc5cccc(-c6nc(-c7ccccc7)nc(-c7ccc(-c8ccc9ccccc9c8)cc7)n6)c5c34)cc2)cc1]

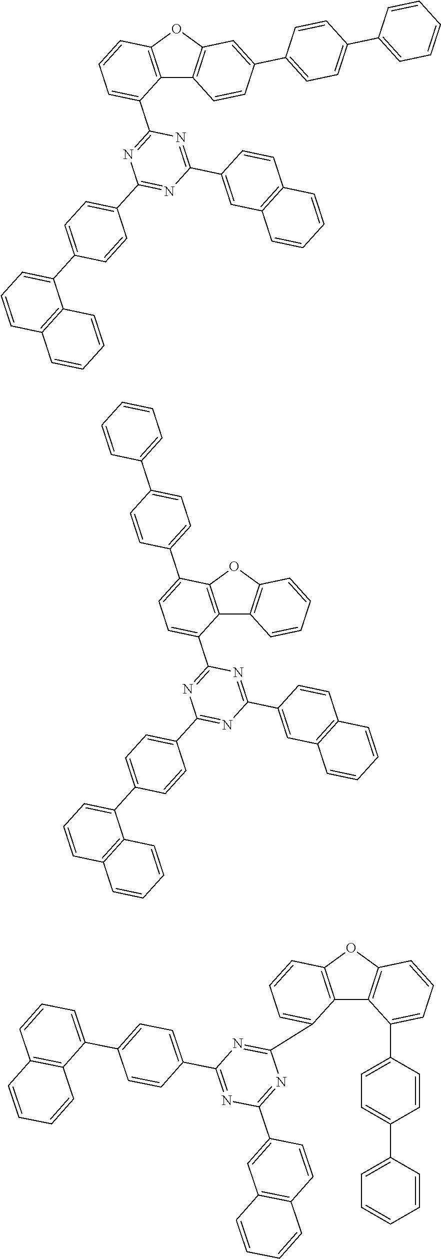 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

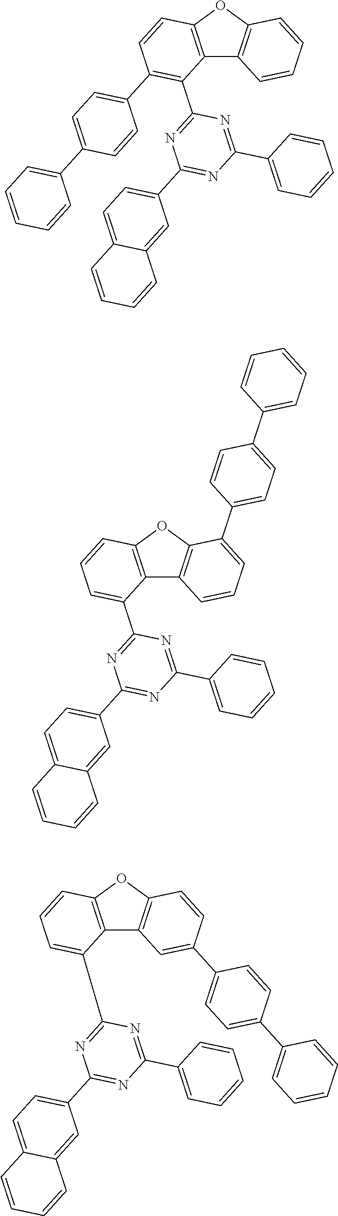 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1]

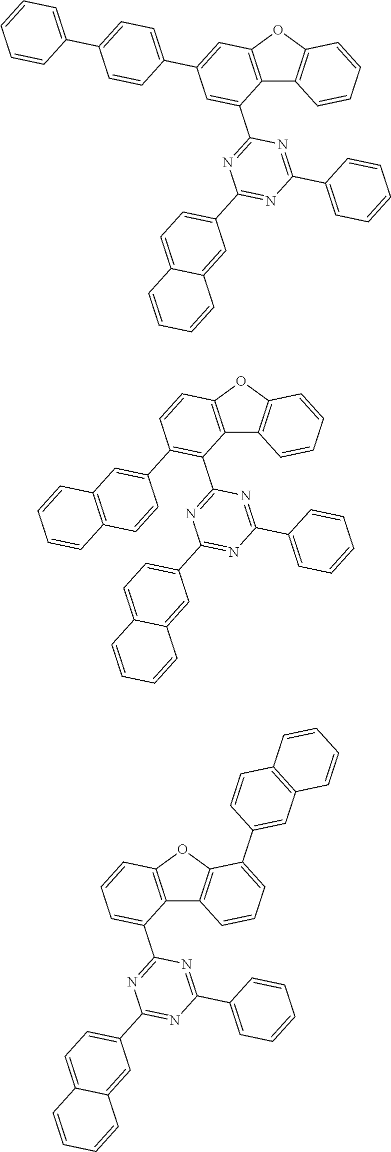 [SMILES: c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1]

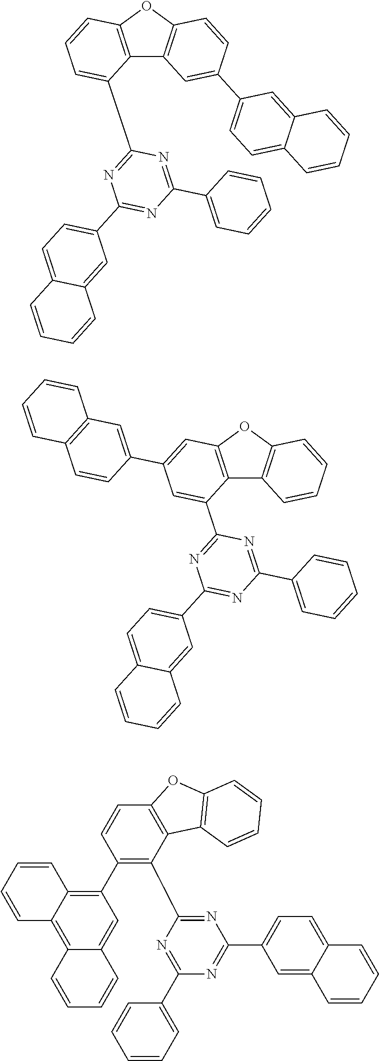

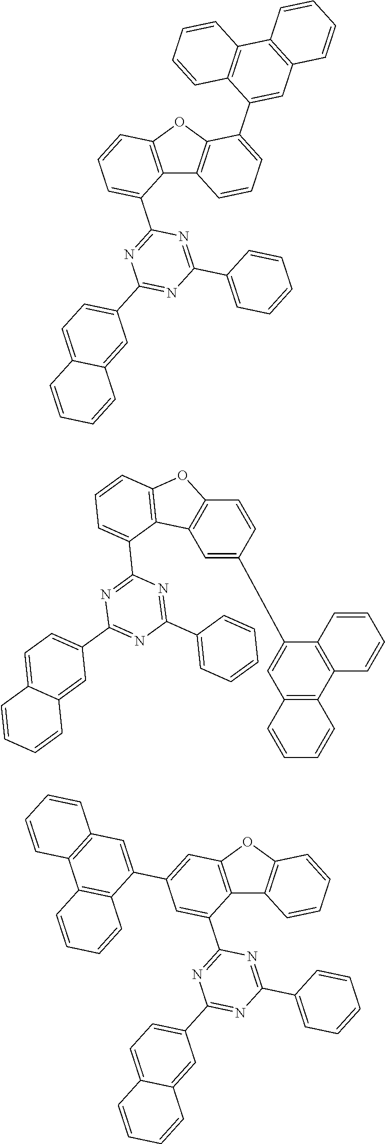

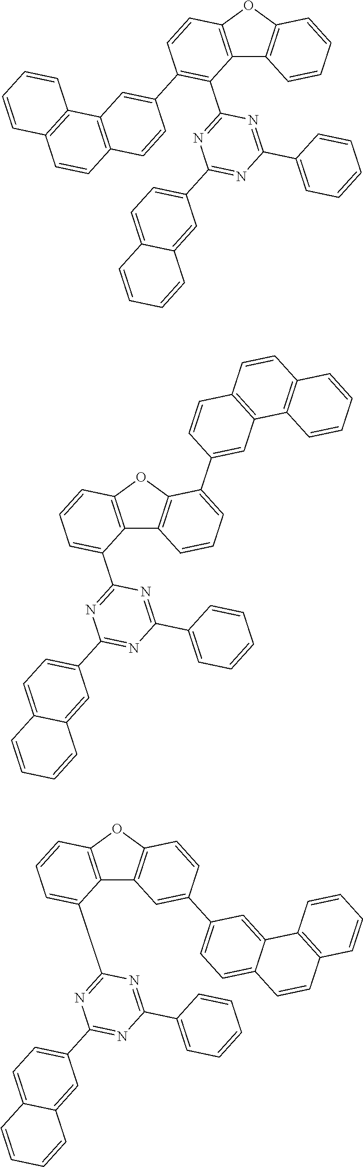 [SMILES: c1ccc(-c2nc(-c3ccc4ccccc4c3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5c(-c6ccc7ccc8ccccc8c7c6)cccc5c34)n2)cc1 ; c1ccc(-c2nc(-c3ccc4ccccc4c3)nc(-c3cccc4oc5ccc(-c6ccc7ccc8ccccc8c7c6)cc5c34)n2)cc1]

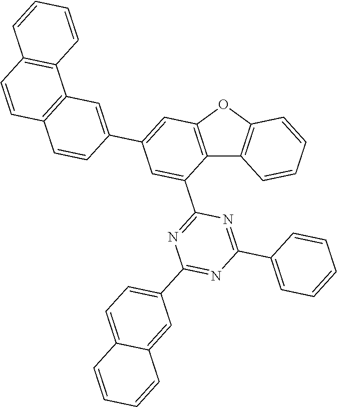 [SMILES: c1ccc(-c2nc(-c3ccc4ccccc4c3)nc(-c3cc(-c4ccc5ccc6ccccc6c5c4)cc4oc5ccccc5c34)n2)cc1]

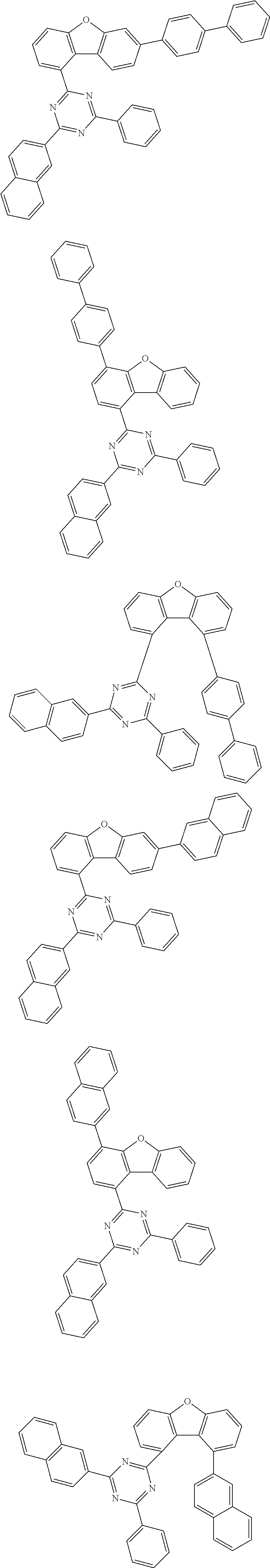

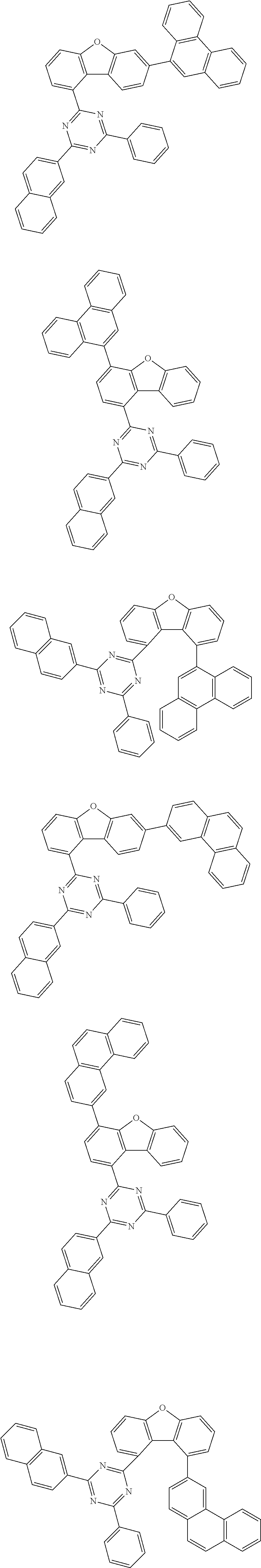

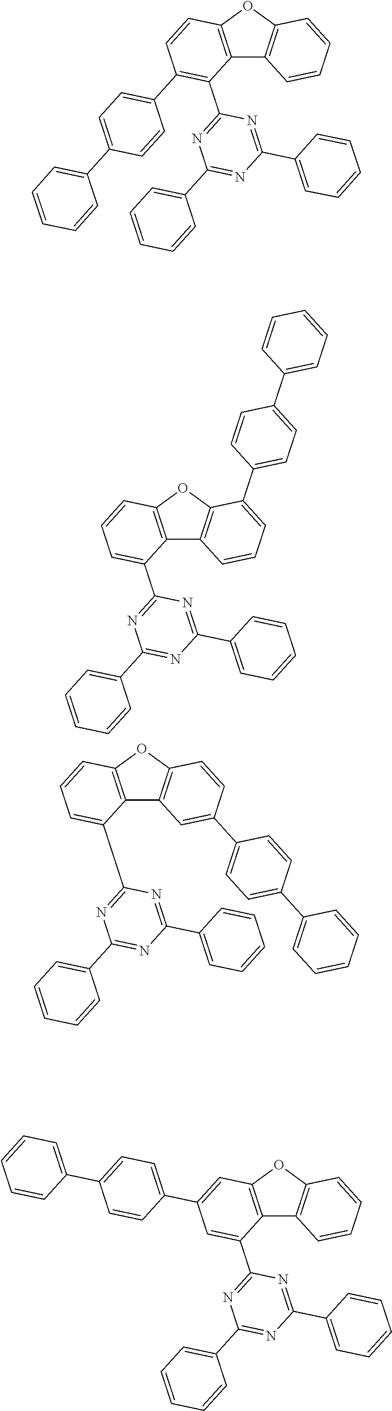

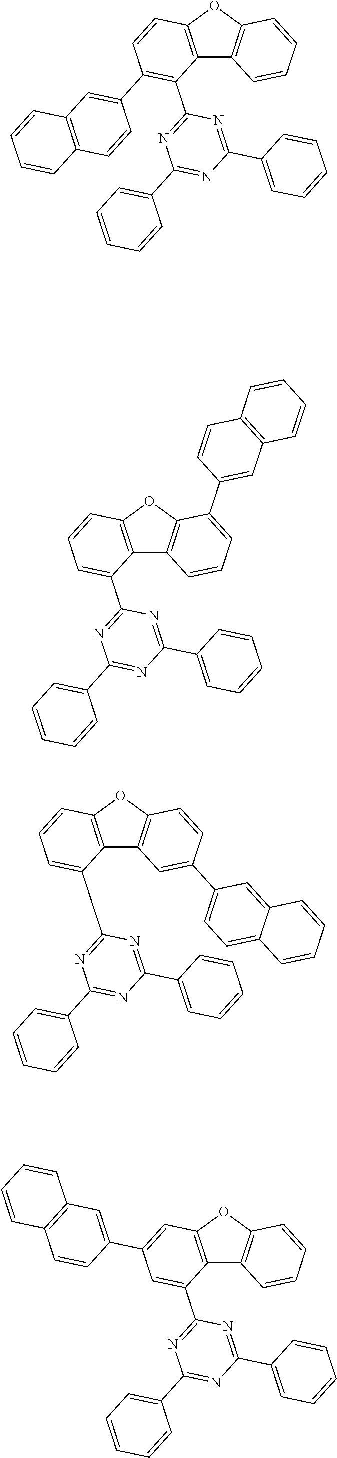 [SMILES: c1ccc(-c2nc(-c3ccccc3)nc(-c3c(-c4ccc5ccccc5c4)ccc4oc5ccccc5c34)n2)cc1 ; c1ccc(-c2nc(-c3ccccc3)nc(-c3cccc4oc5c(-c6ccc7ccccc7c6)cccc5c34)n2)cc1 ; c1ccc(-c2nc(-c3ccccc3)nc(-c3cccc4oc5ccc(-c6ccc7ccccc7c6)cc5c34)n2)cc1 ; c1ccc(-c2nc(-c3ccccc3)nc(-c3cc(-c4ccc5ccccc5c4)cc4oc5ccccc5c34)n2)cc1]

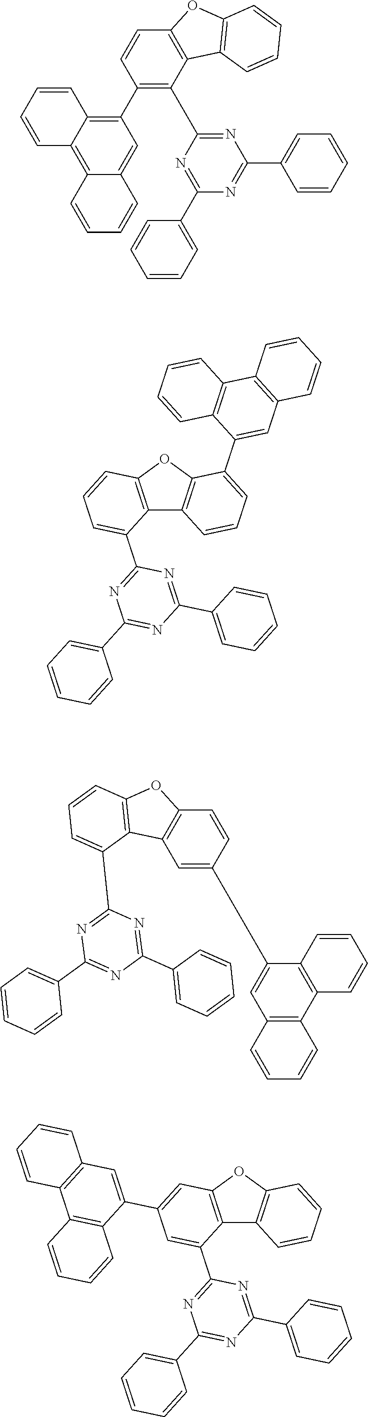 [SMILES: c1ccc(-c2nc(-c3ccccc3)nc(-c3c(-c4cc5ccccc5c5ccccc45)ccc4oc5ccccc5c34)n2)cc1 ; c1ccc(-c2nc(-c3ccccc3)nc(-c3cccc4oc5c(-c6cc7ccccc7c7ccccc67)cccc5c34)n2)cc1 ; c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1]

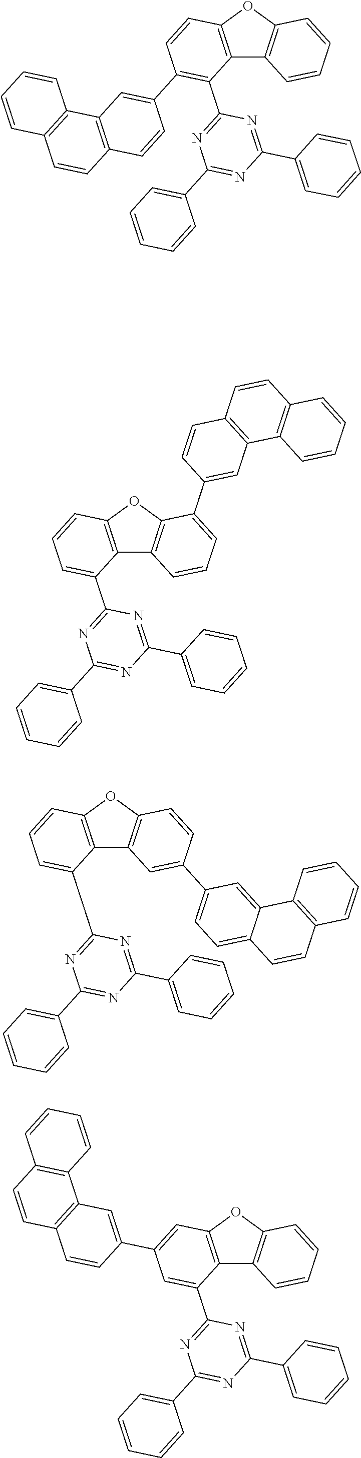

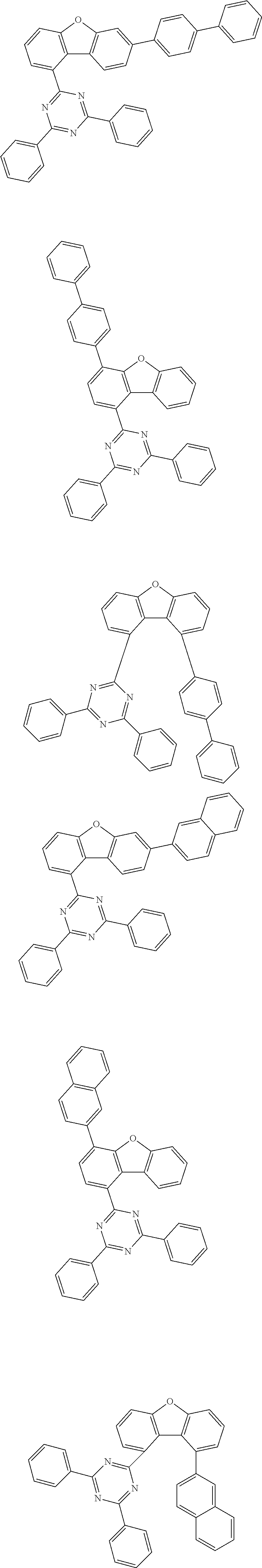

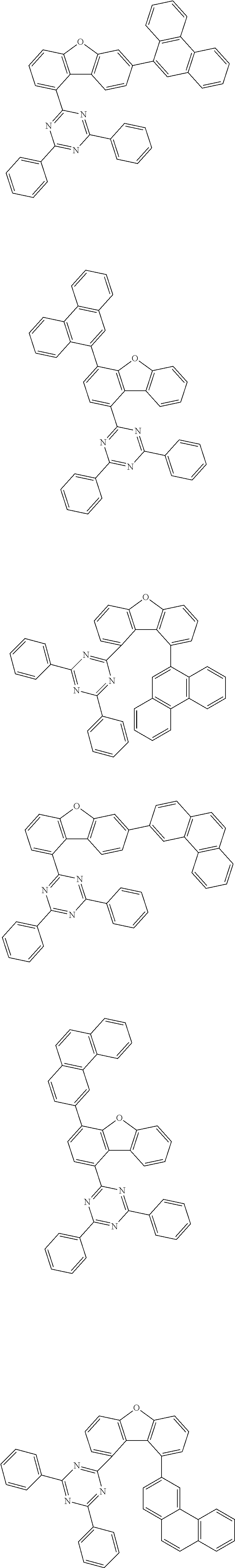 [SMILES: c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3ccc(-c4cc5ccccc5c5ccccc45)c4oc5ccccc5c34)n2)cc1 ; c1ccc(-c2nc(-c3ccccc3)nc(-c3cccc4oc5cccc(-c6cc7ccccc7c7ccccc67)c5c34)n2)cc1 ; c1ccc(-c2nc(-c3ccccc3)nc(-c3cccc4oc5cc(-c6ccc7ccc8ccccc8c7c6)ccc5c34)n2)cc1 ; c1ccc(-c2nc(-c3ccccc3)nc(-c3ccc(-c4ccc5ccc6ccccc6c5c4)c4oc5ccccc5c34)n2)cc1 ; c1ccc(-c2nc(-c3ccccc3)nc(-c3cccc4oc5cccc(-c6ccc7ccc8ccccc8c7c6)c5c34)n2)cc1]

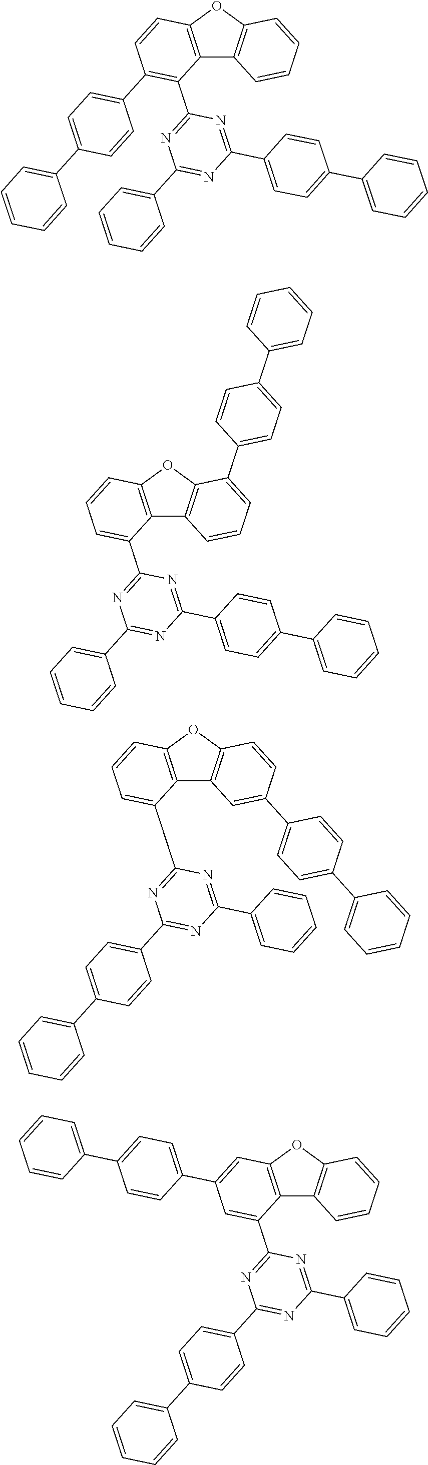

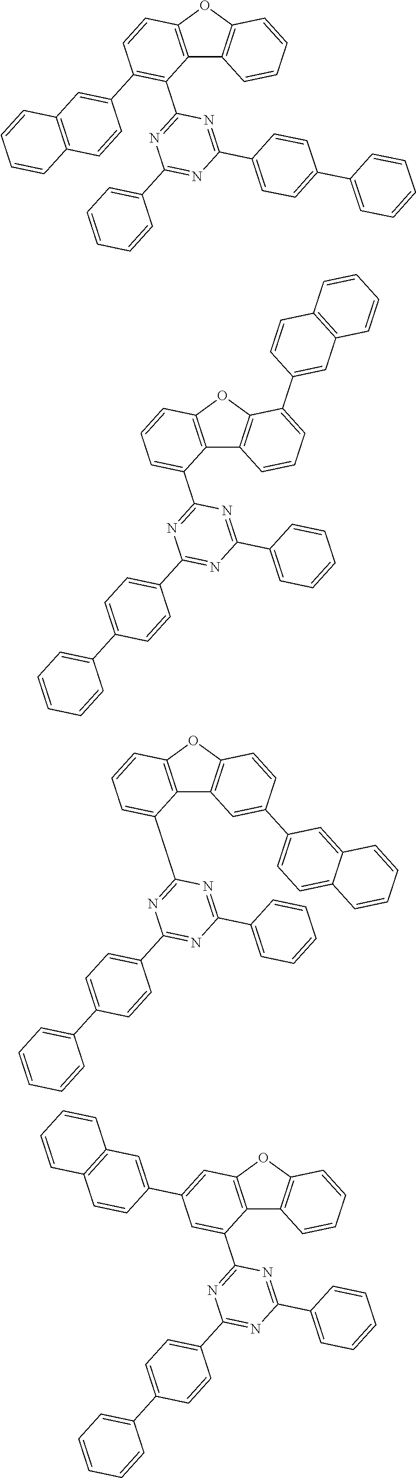

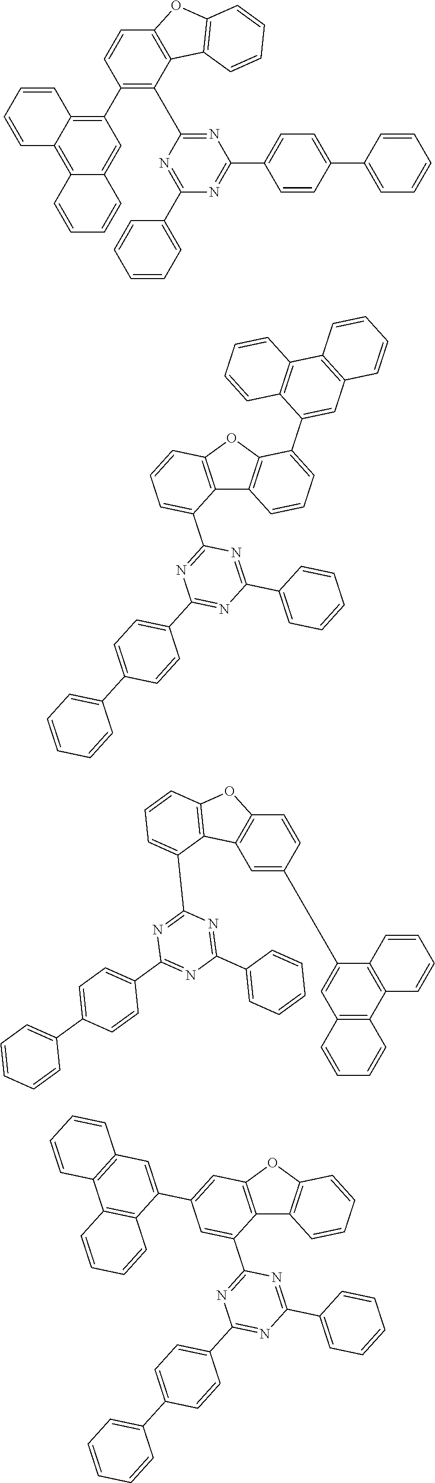 [SMILES: c1ccc(-c2ccc(-c3nc(-c4ccccc4)nc(-c4c(-c5cc6ccccc6c6ccccc56)ccc5oc6ccccc6c45)n3)cc2)cc1 ; c1ccc(-c2ccc(-c3nc(-c4ccccc4)nc(-c4cccc5oc6c(-c7cc8ccccc8c8ccccc78)cccc6c45)n3)cc2)cc1 ; c1ccc(-c2ccc(-c3nc(-c4ccccc4)nc(-c4cccc5oc6ccc(-c7cc8ccccc8c8ccccc78)cc6c45)n3)cc2)cc1 ; c1ccc(-c2ccc(-c3nc(-c4ccccc4)nc(-c4cc(-c5cc6ccccc6c6ccccc56)cc5oc6ccccc6c45)n3)cc2)cc1]

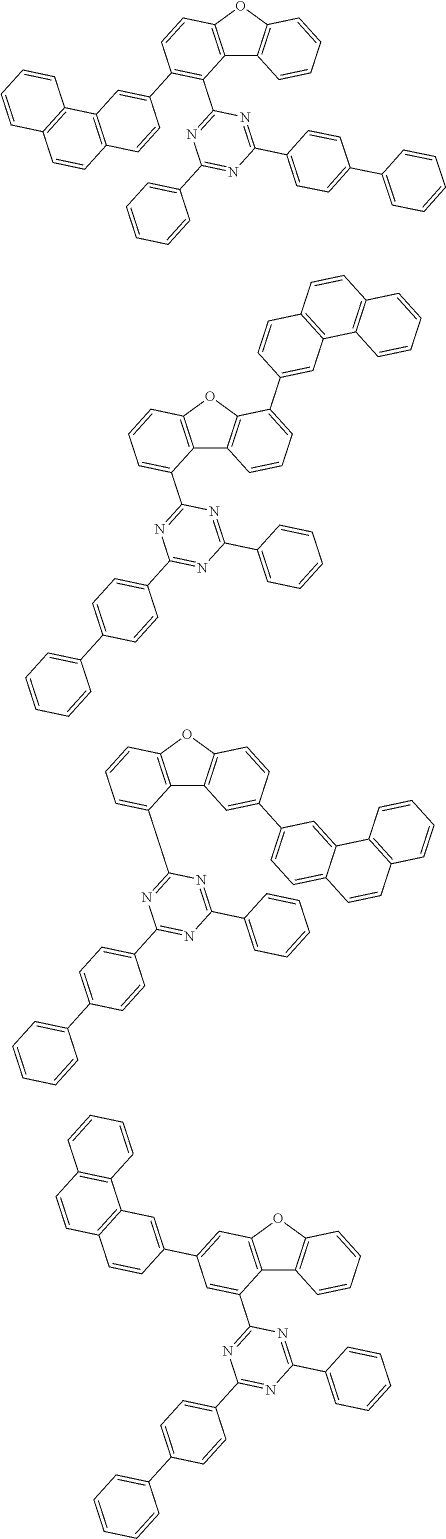

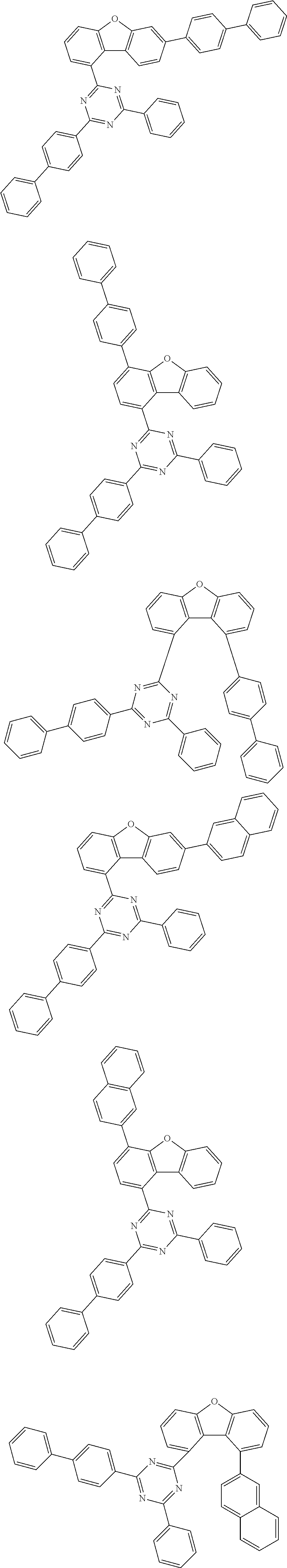

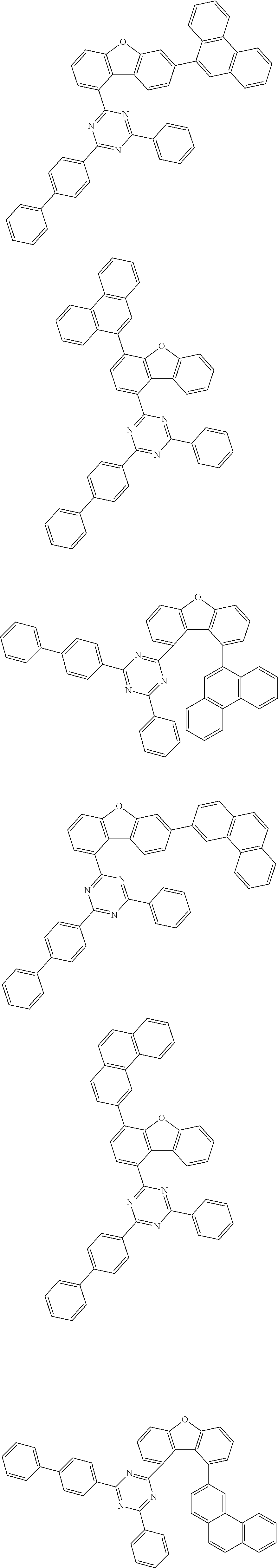

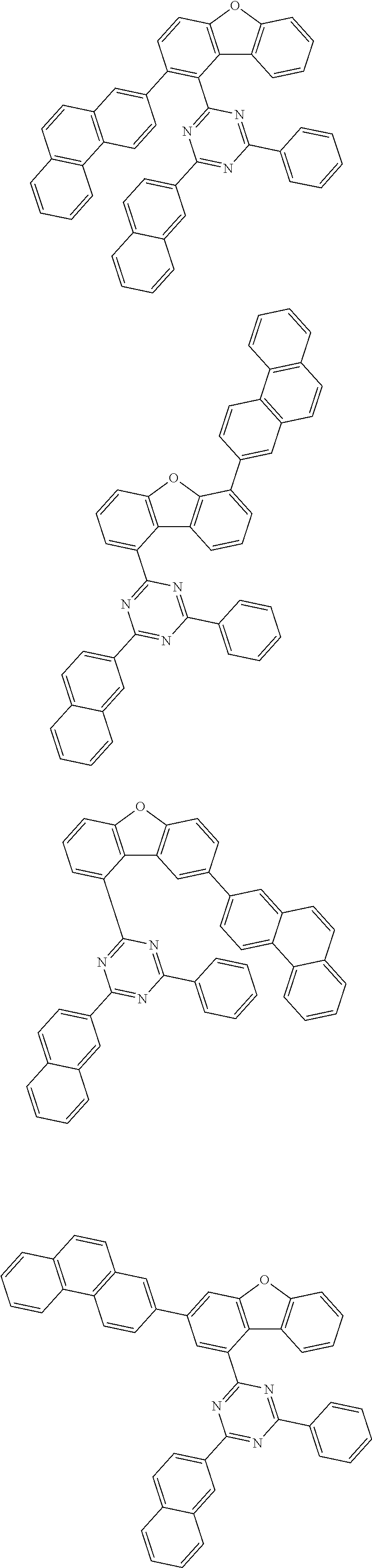 [SMILES: c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1 ; c1ccc(-c2nc(-c3ccc4ccccc4c3)nc(-c3cccc4oc5ccc(-c6ccc7c(ccc8ccccc87)c6)cc5c34)n2)cc1 ; c1ccc(-c2nc(-c3ccc4ccccc4c3)nc(-c3cc(-c4ccc5c(ccc6ccccc65)c4)cc4oc5ccccc5c34)n2)cc1]

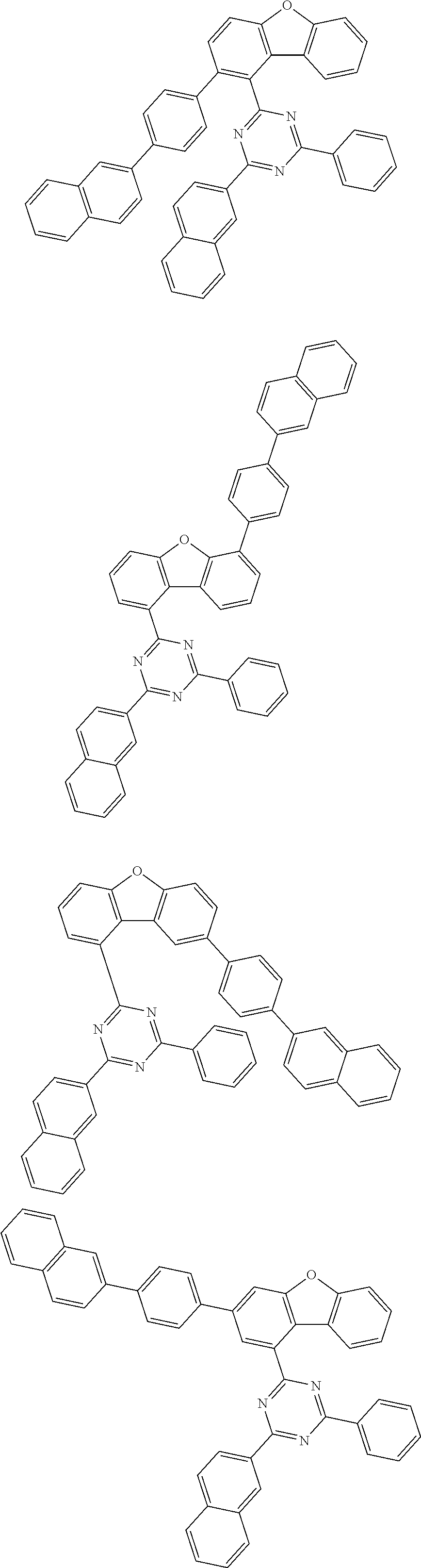

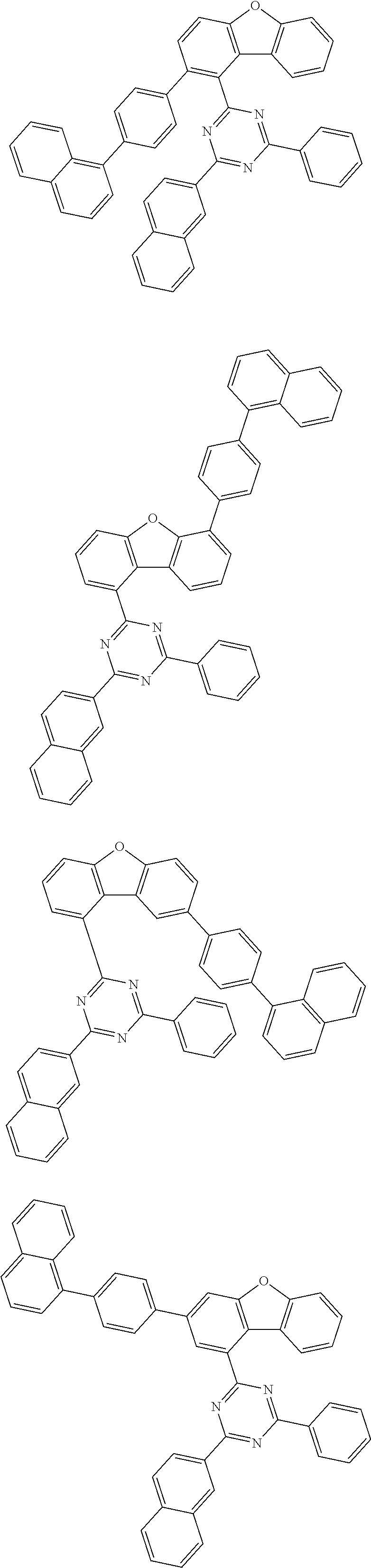

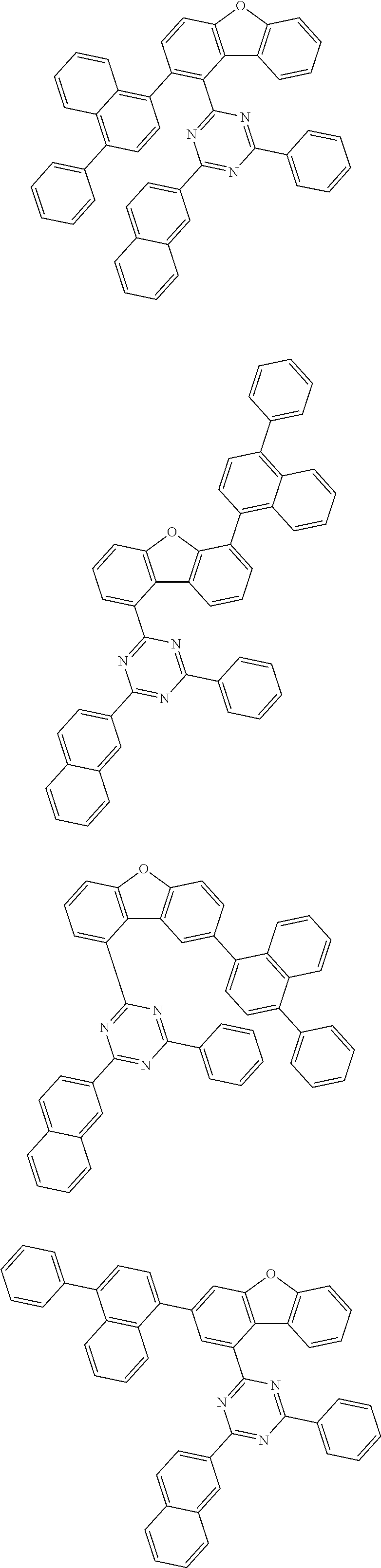 [SMILES: c1ccc(-c2nc(-c3ccc4ccccc4c3)nc(-c3c(-c4ccc(-c5ccccc5)c5ccccc45)ccc4oc5ccccc5c34)n2)cc1 ; c1ccc(-c2nc(-c3ccc4ccccc4c3)nc(-c3cccc4oc5c(-c6ccc(-c7ccccc7)c7ccccc67)cccc5c34)n2)cc1 ; c1ccc(-c2nc(-c3ccc4ccccc4c3)nc(-c3cccc4oc5ccc(-c6ccc(-c7ccccc7)c7ccccc67)cc5c34)n2)cc1 ; c1ccc(-c2nc(-c3ccc4ccccc4c3)nc(-c3cc(-c4ccc(-c5ccccc5)c5ccccc45)cc4oc5ccccc5c34)n2)cc1]

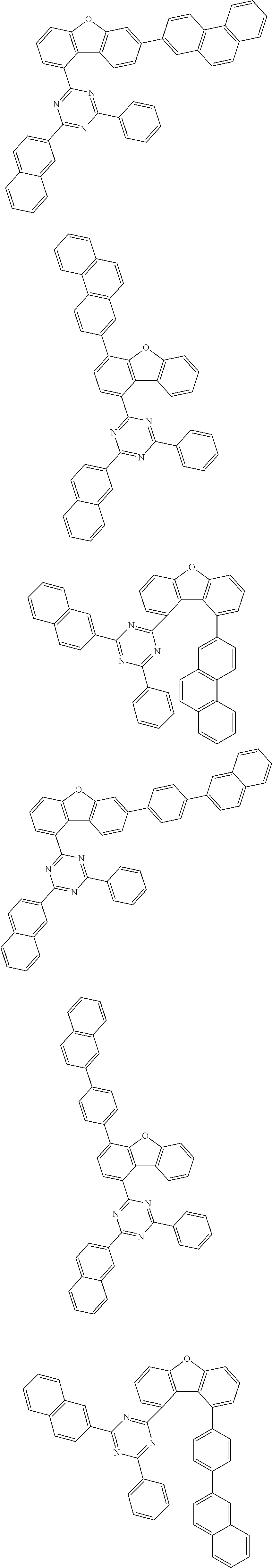

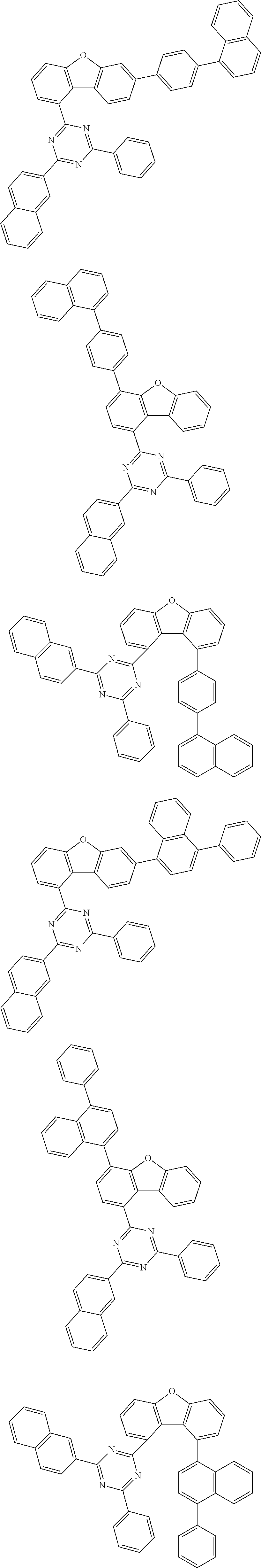

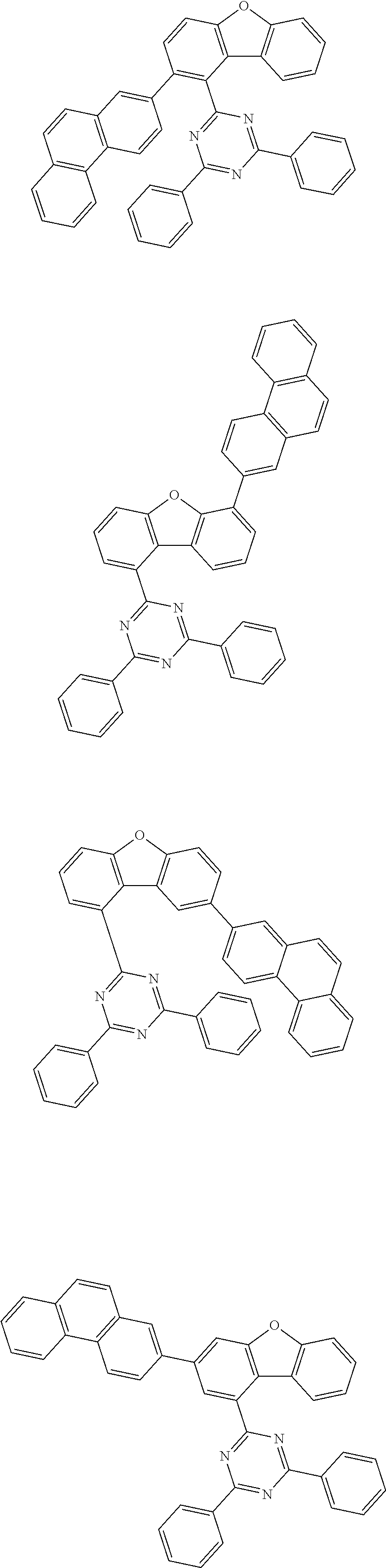 [SMILES: c1ccc(-c2nc(-c3ccccc3)nc(-c3c(-c4ccc5c(ccc6ccccc65)c4)ccc4oc5ccccc5c34)n2)cc1 ; c1ccc(-c2nc(-c3ccccc3)nc(-c3cccc4oc5c(-c6ccc7c(ccc8ccccc87)c6)cccc5c34)n2)cc1 ; c1ccc(-c2nc(-c3ccccc3)nc(-c3cccc4oc5ccc(-c6ccc7c(ccc8ccccc87)c6)cc5c34)n2)cc1 ; c1ccc(-c2nc(-c3ccccc3)nc(-c3cc(-c4ccc5c(ccc6ccccc65)c4)cc4oc5ccccc5c34)n2)cc1]

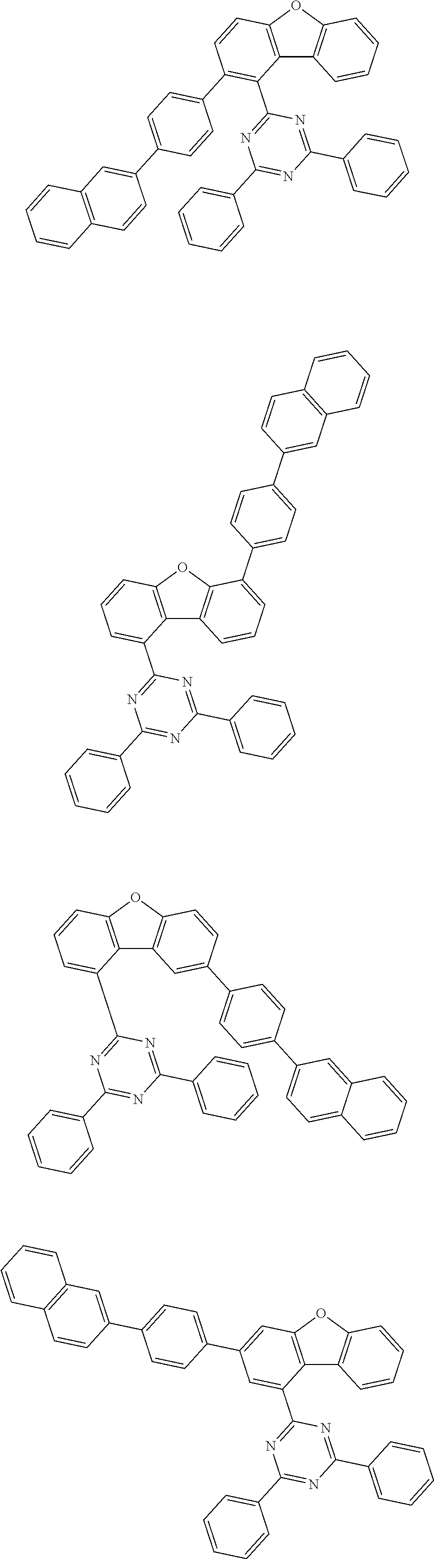 [SMILES: c1ccc(-c2nc(-c3ccccc3)nc(-c3c(-c4ccc(-c5ccc6ccccc6c5)cc4)ccc4oc5ccccc5c34)n2)cc1 ; c1ccc(-c2nc(-c3ccccc3)nc(-c3cccc4oc5c(-c6ccc(-c7ccc8ccccc8c7)cc6)cccc5c34)n2)cc1 ; c1ccc(-c2nc(-c3ccccc3)nc(-c3cccc4oc5ccc(-c6ccc(-c7ccc8ccccc8c7)cc6)cc5c34)n2)cc1 ; c1ccc(-c2nc(-c3ccccc3)nc(-c3cc(-c4ccc(-c5ccc6ccccc6c5)cc4)cc4oc5ccccc5c34)n2)cc1]

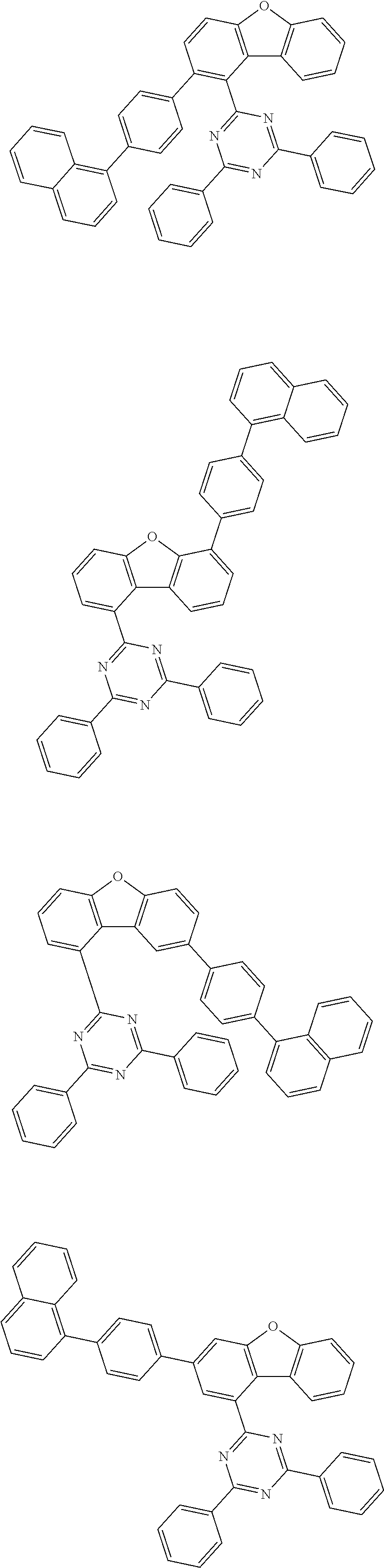 [SMILES: c1ccc(-c2nc(-c3ccccc3)nc(-c3c(-c4ccc(-c5cccc6ccccc56)cc4)ccc4oc5ccccc5c34)n2)cc1 ; c1ccc(-c2nc(-c3ccccc3)nc(-c3cccc4oc5c(-c6ccc(-c7cccc8ccccc78)cc6)cccc5c34)n2)cc1 ; c1ccc(-c2nc(-c3ccccc3)nc(-c3cccc4oc5ccc(-c6ccc(-c7cccc8ccccc78)cc6)cc5c34)n2)cc1 ; c1ccc(-c2nc(-c3ccccc3)nc(-c3cc(-c4ccc(-c5cccc6ccccc56)cc4)cc4oc5ccccc5c34)n2)cc1]

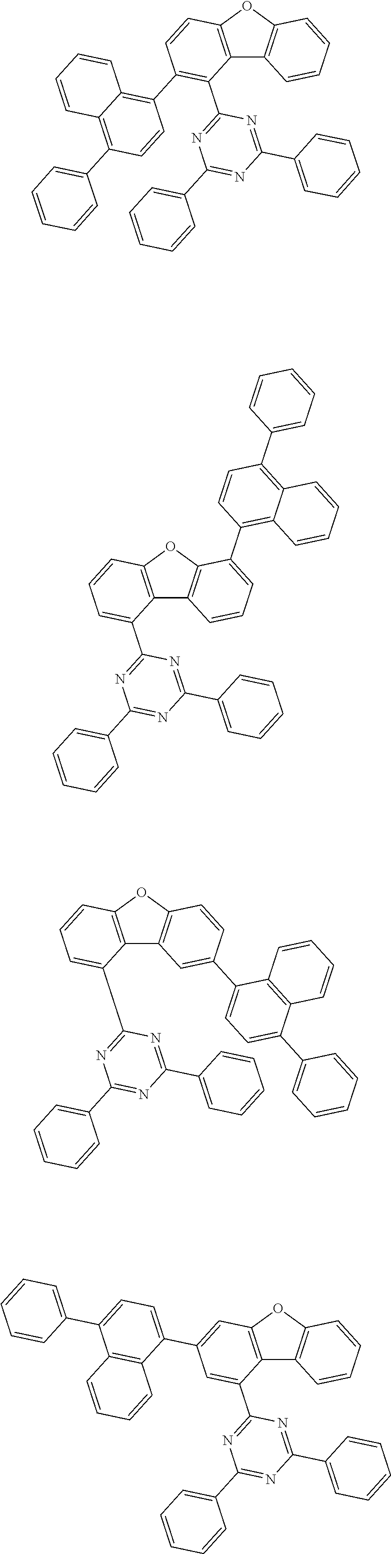 [SMILES: c1ccc(-c2nc(-c3ccccc3)nc(-c3c(-c4ccc(-c5ccccc5)c5ccccc45)ccc4oc5ccccc5c34)n2)cc1 ; c1ccc(-c2nc(-c3ccccc3)nc(-c3cccc4oc5c(-c6ccc(-c7ccccc7)c7ccccc67)cccc5c34)n2)cc1 ; c1ccc(-c2nc(-c3ccccc3)nc(-c3cccc4oc5ccc(-c6ccc(-c7ccccc7)c7ccccc67)cc5c34)n2)cc1 ; c1ccc(-c2nc(-c3ccccc3)nc(-c3cc(-c4ccc(-c5ccccc5)c5ccccc45)cc4oc5ccccc5c34)n2)cc1]

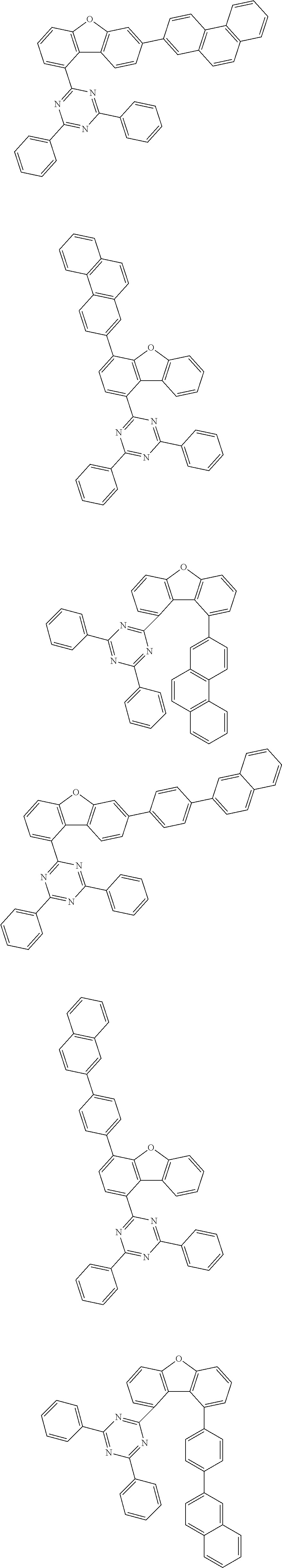

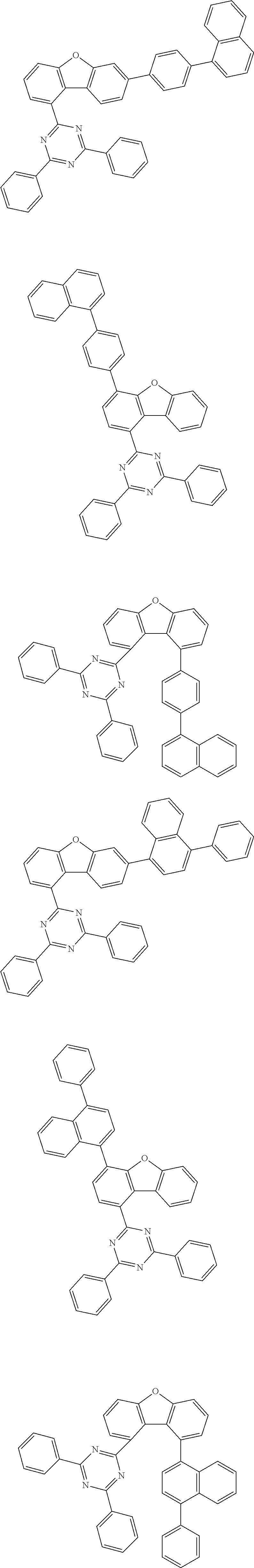

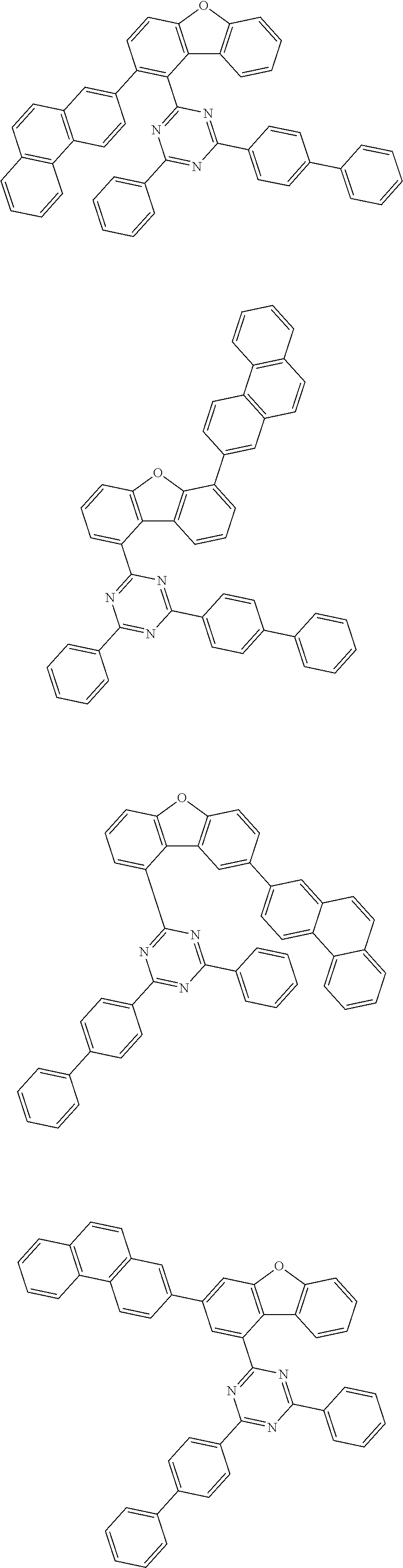 [SMILES: c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1 ; c1ccc(-c2ccc(-c3nc(-c4ccccc4)nc(-c4cccc5oc6ccc(-c7ccc8c(ccc9ccccc98)c7)cc6c45)n3)cc2)cc1 ; c1ccc(-c2ccc(-c3nc(-c4ccccc4)nc(-c4cc(-c5ccc6c(ccc7ccccc76)c5)cc5oc6ccccc6c45)n3)cc2)cc1]

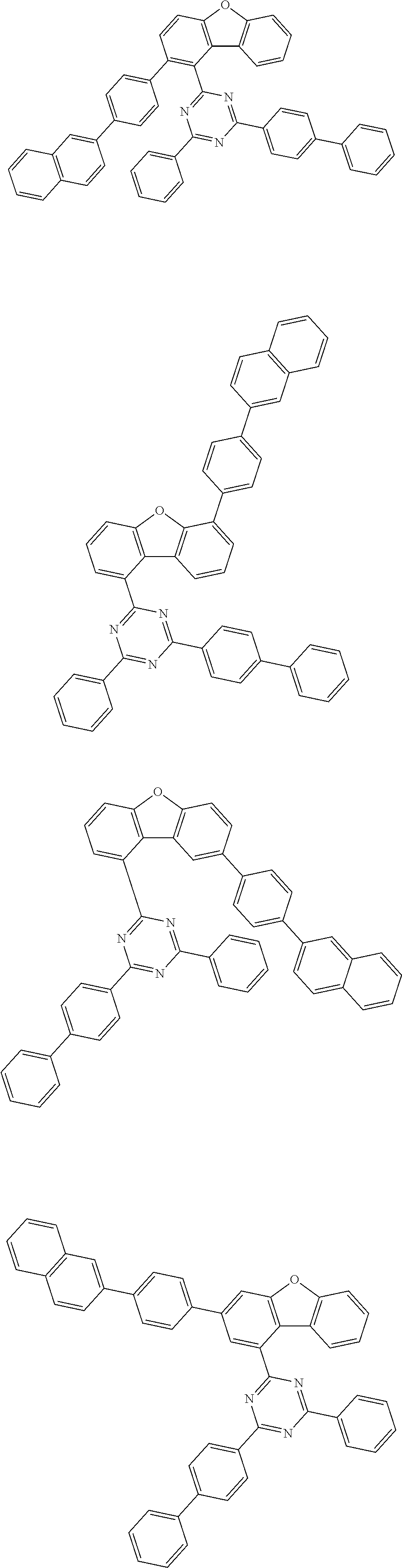 [SMILES: c1ccc(-c2ccc(-c3nc(-c4ccccc4)nc(-c4c(-c5ccc(-c6ccc7ccccc7c6)cc5)ccc5oc6ccccc6c45)n3)cc2)cc1 ; c1ccc(-c2ccc(-c3nc(-c4ccccc4)nc(-c4cccc5oc6c(-c7ccc(-c8ccc9ccccc9c8)cc7)cccc6c45)n3)cc2)cc1 ; c1ccc(-c2ccc(-c3nc(-c4ccccc4)nc(-c4cccc5oc6ccc(-c7ccc(-c8ccc9ccccc9c8)cc7)cc6c45)n3)cc2)cc1 ; c1ccc(-c2ccc(-c3nc(-c4ccccc4)nc(-c4cc(-c5ccc(-c6ccc7ccccc7c6)cc5)cc5oc6ccccc6c45)n3)cc2)cc1]

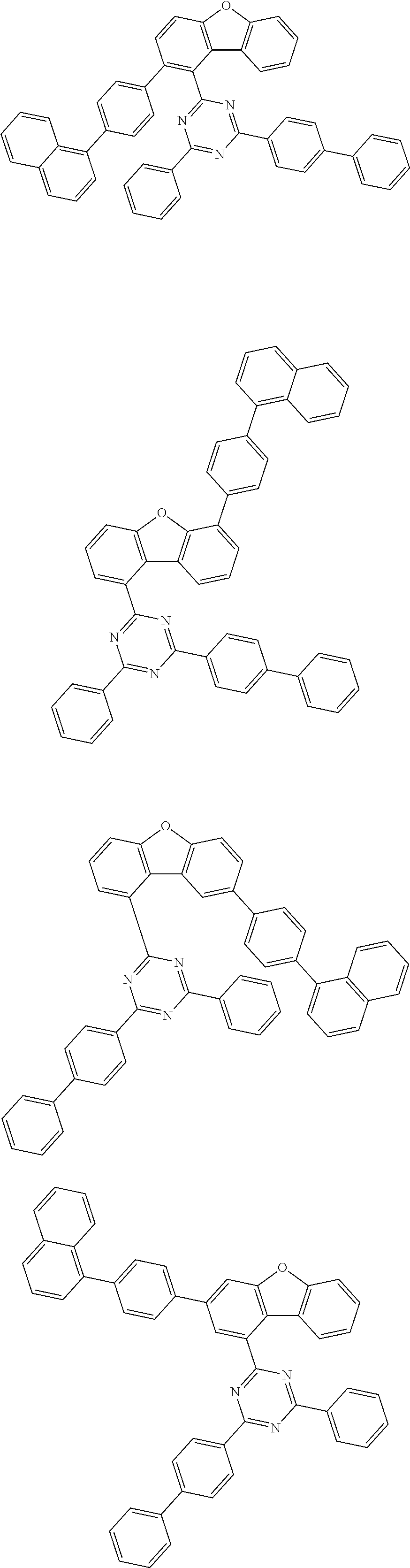

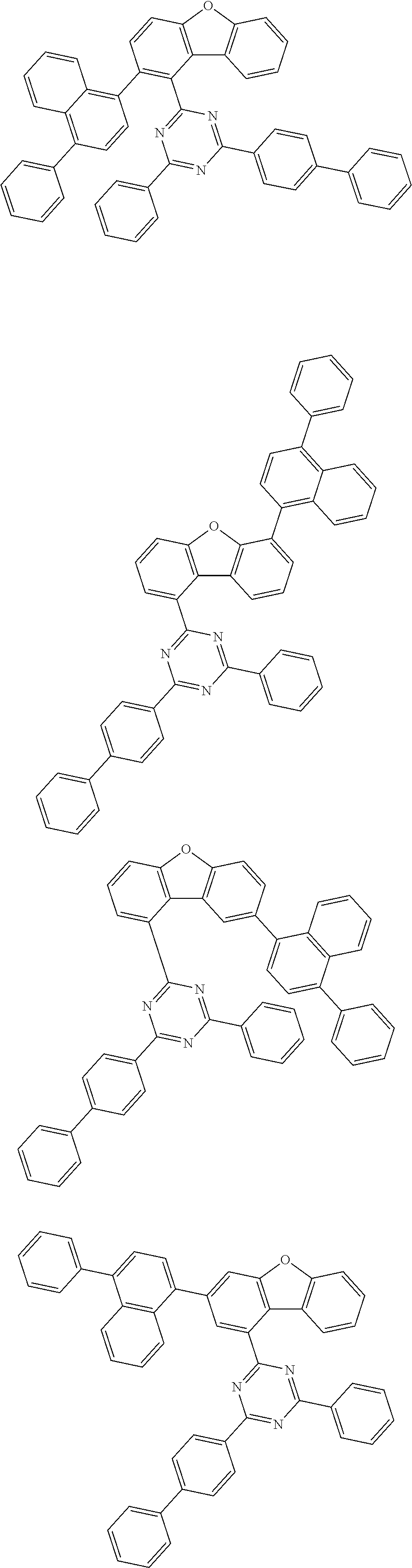 [SMILES: c1ccc(-c2ccc(-c3nc(-c4ccccc4)nc(-c4c(-c5ccc(-c6ccccc6)c6ccccc56)ccc5oc6ccccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1 ; c1ccc(-c2ccc(-c3nc(-c4ccccc4)nc(-c4cccc5oc6ccc(-c7ccc(-c8ccccc8)c8ccccc78)cc6c45)n3)cc2)cc1 ; c1ccc(-c2ccc(-c3nc(-c4ccccc4)nc(-c4cc(-c5ccc(-c6ccccc6)c6ccccc56)cc5oc6ccccc6c45)n3)cc2)cc1]

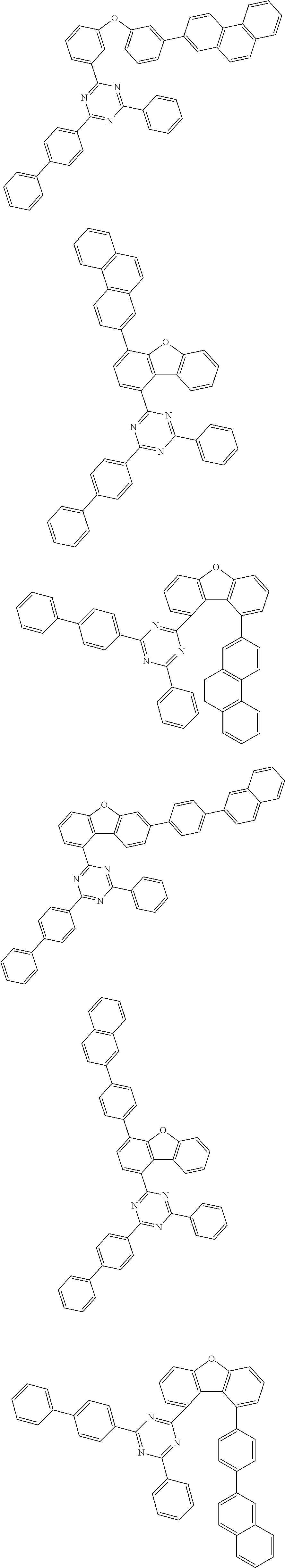

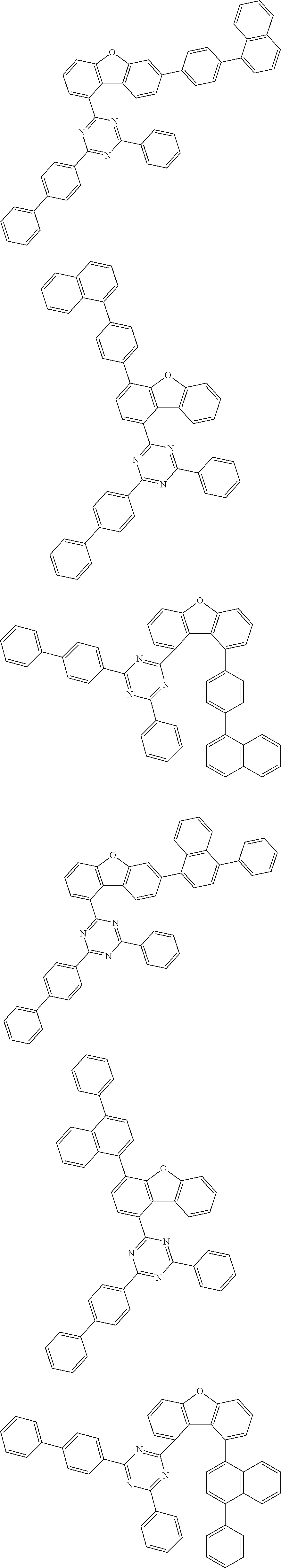

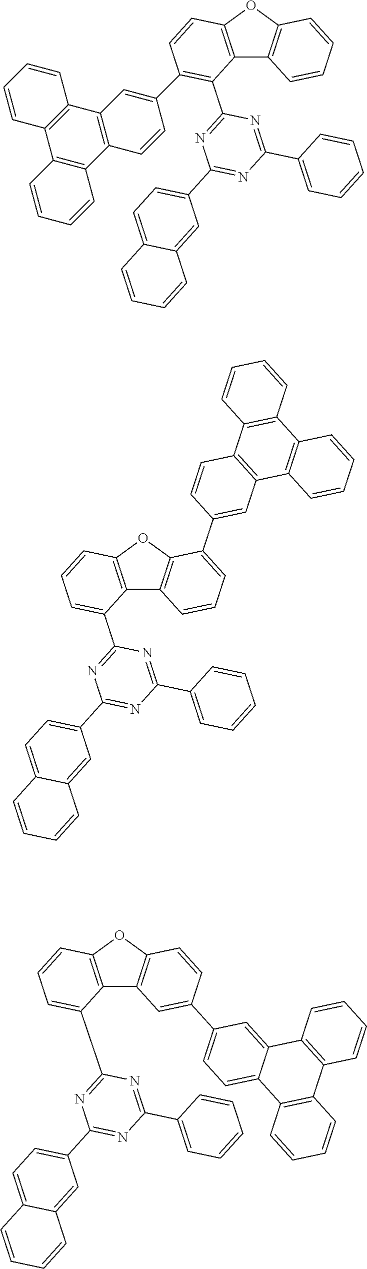 [SMILES: c1ccc(-c2nc(-c3ccc4ccccc4c3)nc(-c3c(-c4ccc5c6ccccc6c6ccccc6c5c4)ccc4oc5ccccc5c34)n2)cc1 ; c1ccc(-c2nc(-c3ccc4ccccc4c3)nc(-c3cccc4oc5c(-c6ccc7c8ccccc8c8ccccc8c7c6)cccc5c34)n2)cc1 ; c1ccc(-c2nc(-c3ccc4ccccc4c3)nc(-c3cccc4oc5ccc(-c6ccc7c8ccccc8c8ccccc8c7c6)cc5c34)n2)cc1]

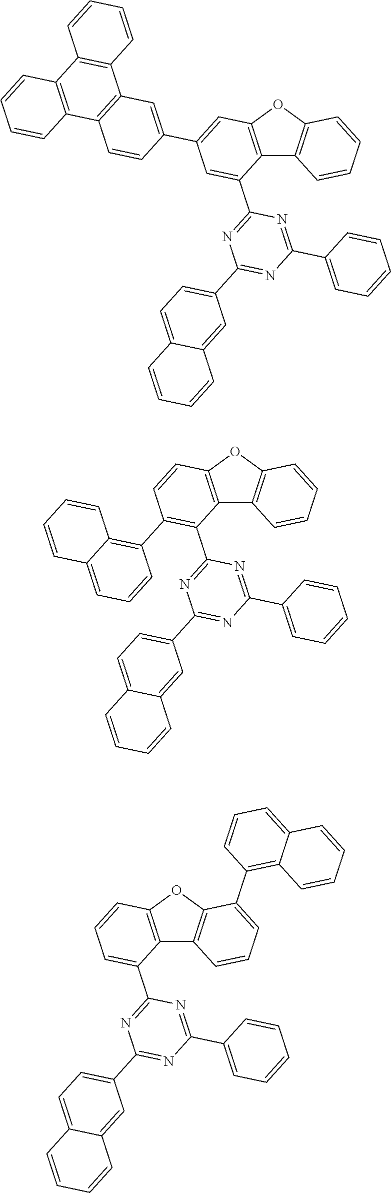 [SMILES: c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3c(-c4cccc5ccccc45)ccc4oc5ccccc5c34)n2)cc1 ; c1ccc(-c2nc(-c3ccc4ccccc4c3)nc(-c3cccc4oc5c(-c6cccc7ccccc67)cccc5c34)n2)cc1]

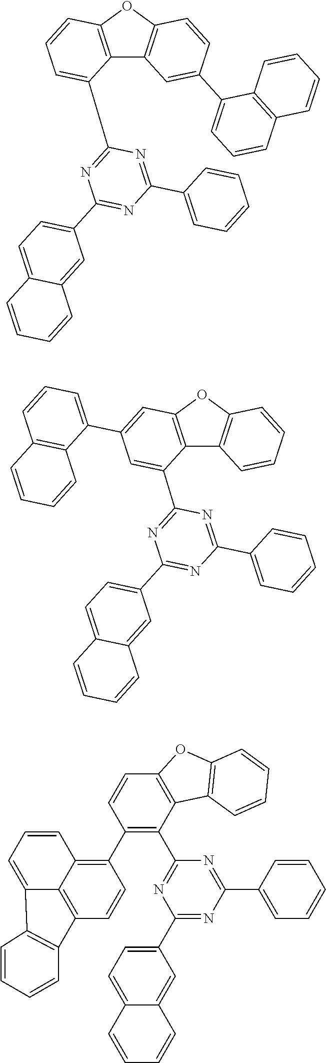

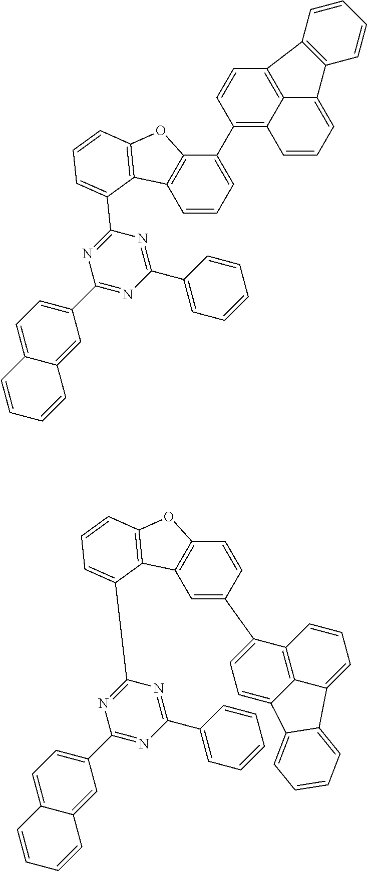 [SMILES: c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2nc(-c3ccc4ccccc4c3)nc(-c3cccc4oc5ccc(-c6ccc7c8c(cccc68)-c6ccccc6-7)cc5c34)n2)cc1]

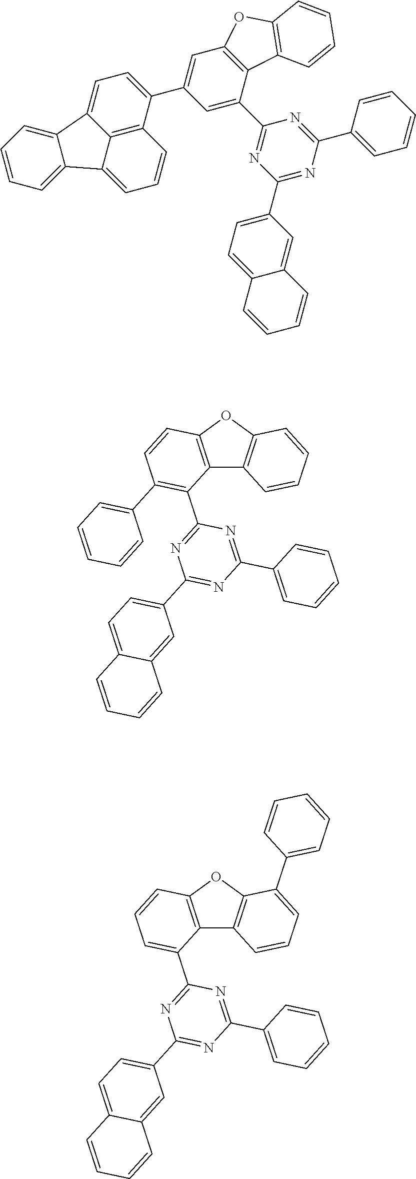 [SMILES: c1ccc(-c2nc(-c3ccc4ccccc4c3)nc(-c3cc(-c4ccc5c6c(cccc46)-c4ccccc4-5)cc4oc5ccccc5c34)n2)cc1 ; c1ccc(-c2nc(-c3ccc4ccccc4c3)nc(-c3c(-c4ccccc4)ccc4oc5ccccc5c34)n2)cc1 ; c1ccc(-c2nc(-c3ccc4ccccc4c3)nc(-c3cccc4oc5c(-c6ccccc6)cccc5c34)n2)cc1]

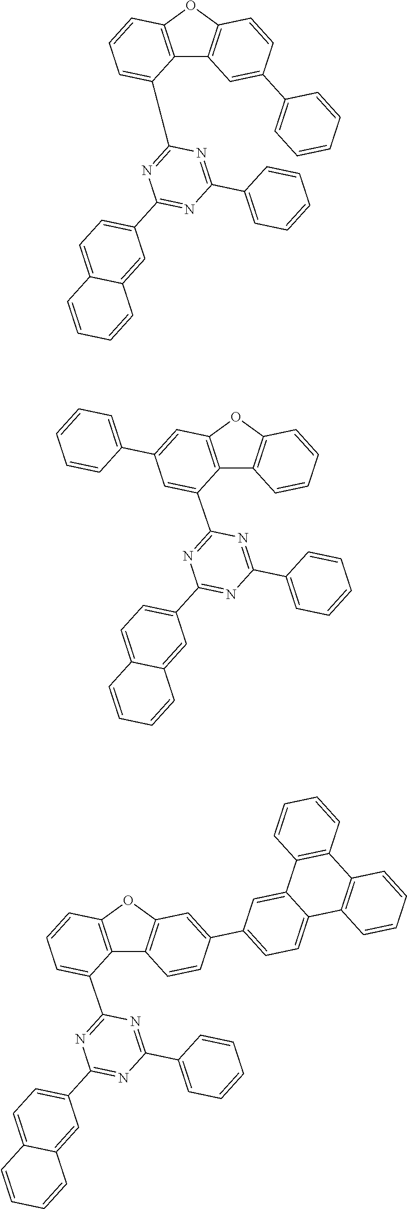 [SMILES: c1ccc(-c2ccc3oc4cccc(-c5nc(-c6ccccc6)nc(-c6ccc7ccccc7c6)n5)c4c3c2)cc1 ; c1ccc(-c2cc(-c3nc(-c4ccccc4)nc(-c4ccc5ccccc5c4)n3)c3c(c2)oc2ccccc23)cc1 ; c1ccc(-c2nc(-c3ccc4ccccc4c3)nc(-c3cccc4oc5cc(-c6ccc7c8ccccc8c8ccccc8c7c6)ccc5c34)n2)cc1]

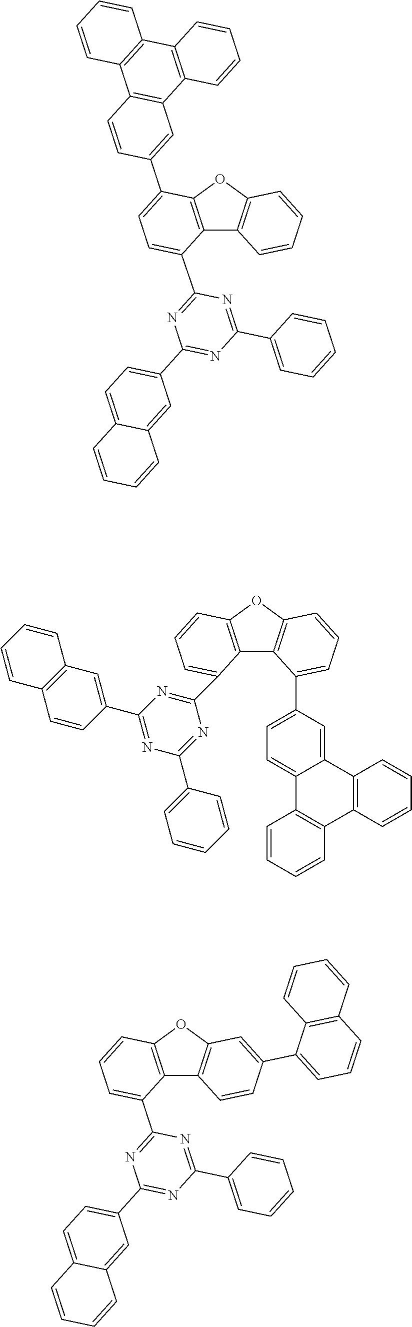 [SMILES: c1ccc(-c2nc(-c3ccc4ccccc4c3)nc(-c3ccc(-c4ccc5c6ccccc6c6ccccc6c5c4)c4oc5ccccc5c34)n2)cc1 ; c1ccc(-c2nc(-c3ccc4ccccc4c3)nc(-c3cccc4oc5cccc(-c6ccc7c8ccccc8c8ccccc8c7c6)c5c34)n2)cc1 ; c1ccc(-c2nc(-c3ccc4ccccc4c3)nc(-c3cccc4oc5cc(-c6cccc7ccccc67)ccc5c34)n2)cc1]

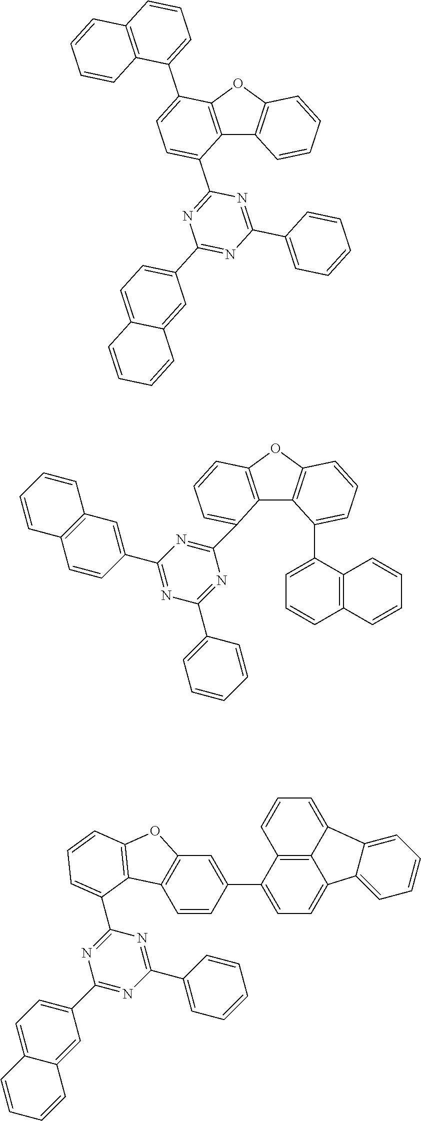 [SMILES: c1ccc(-c2nc(-c3ccc4ccccc4c3)nc(-c3ccc(-c4cccc5ccccc45)c4oc5ccccc5c34)n2)cc1 ; c1ccc(-c2nc(-c3ccc4ccccc4c3)nc(-c3cccc4oc5cccc(-c6cccc7ccccc67)c5c34)n2)cc1 ; c1ccc(-c2nc(-c3ccc4ccccc4c3)nc(-c3cccc4oc5cc(-c6ccc7c8c(cccc68)-c6ccccc6-7)ccc5c34)n2)cc1]

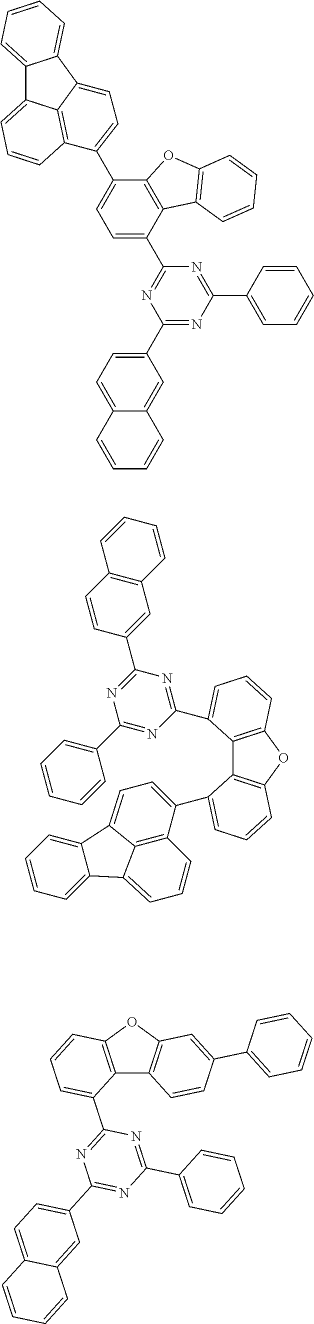 [SMILES: c1ccc(-c2nc(-c3ccc4ccccc4c3)nc(-c3ccc(-c4ccc5c6c(cccc46)-c4ccccc4-5)c4oc5ccccc5c34)n2)cc1 ; c1ccc(-c2nc(-c3ccc4ccccc4c3)nc(-c3cccc4oc5cccc(-c6ccc7c8c(cccc68)-c6ccccc6-7)c5c34)n2)cc1 ; c1ccc(-c2ccc3c(c2)oc2cccc(-c4nc(-c5ccccc5)nc(-c5ccc6ccccc6c5)n4)c23)cc1]

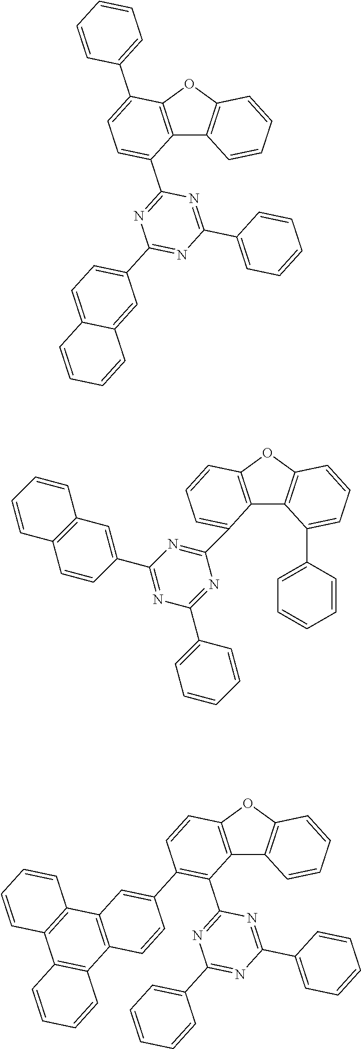 [SMILES: c1ccc(-c2nc(-c3ccc4ccccc4c3)nc(-c3ccc(-c4ccccc4)c4oc5ccccc5c34)n2)cc1 ; c1ccc(-c2nc(-c3ccc4ccccc4c3)nc(-c3cccc4oc5cccc(-c6ccccc6)c5c34)n2)cc1 ; c1ccc(-c2nc(-c3ccccc3)nc(-c3c(-c4ccc5c6ccccc6c6ccccc6c5c4)ccc4oc5ccccc5c34)n2)cc1]

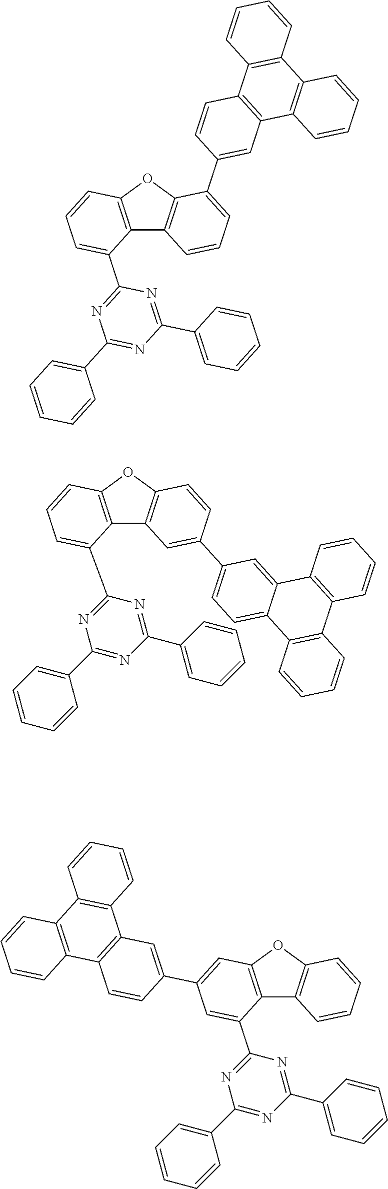 [SMILES: c1ccc(-c2nc(-c3ccccc3)nc(-c3cccc4oc5c(-c6ccc7c8ccccc8c8ccccc8c7c6)cccc5c34)n2)cc1 ; c1ccc(-c2nc(-c3ccccc3)nc(-c3cccc4oc5ccc(-c6ccc7c8ccccc8c8ccccc8c7c6)cc5c34)n2)cc1 ; c1ccc(-c2nc(-c3ccccc3)nc(-c3cc(-c4ccc5c6ccccc6c6ccccc6c5c4)cc4oc5ccccc5c34)n2)cc1]

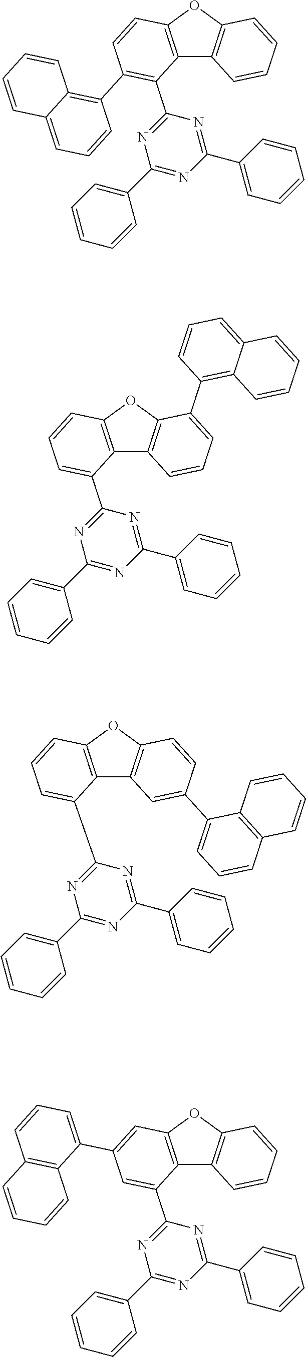 [SMILES: c1ccc(-c2nc(-c3ccccc3)nc(-c3c(-c4cccc5ccccc45)ccc4oc5ccccc5c34)n2)cc1 ; c1ccc(-c2nc(-c3ccccc3)nc(-c3cccc4oc5c(-c6cccc7ccccc67)cccc5c34)n2)cc1 ; c1ccc(-c2nc(-c3ccccc3)nc(-c3cccc4oc5ccc(-c6cccc7ccccc67)cc5c34)n2)cc1 ; c1ccc(-c2nc(-c3ccccc3)nc(-c3cc(-c4cccc5ccccc45)cc4oc5ccccc5c34)n2)cc1]

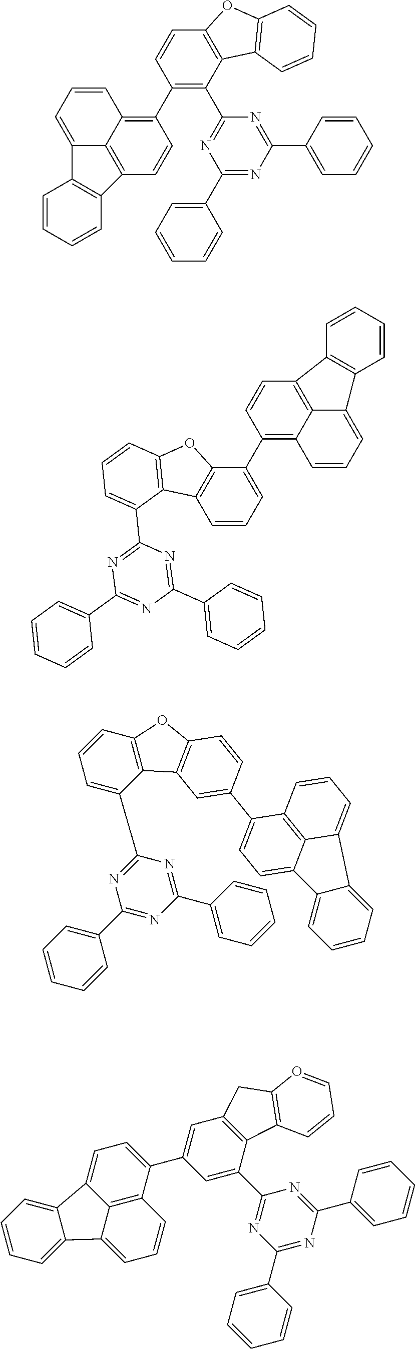

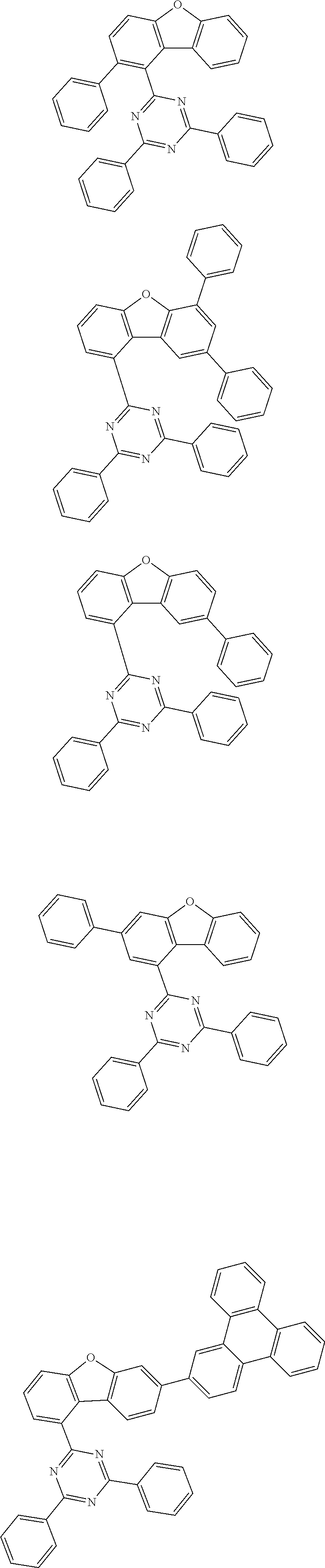 [SMILES: c1ccc(-c2nc(-c3ccccc3)nc(-c3c(-c4ccccc4)ccc4oc5ccccc5c34)n2)cc1 ; c1ccc(-c2cc(-c3ccccc3)c3oc4cccc(-c5nc(-c6ccccc6)nc(-c6ccccc6)n5)c4c3c2)cc1 ; c1ccc(-c2ccc3oc4cccc(-c5nc(-c6ccccc6)nc(-c6ccccc6)n5)c4c3c2)cc1 ; c1ccc(-c2cc(-c3nc(-c4ccccc4)nc(-c4ccccc4)n3)c3c(c2)oc2ccccc23)cc1 ; c1ccc(-c2nc(-c3ccccc3)nc(-c3cccc4oc5cc(-c6ccc7c8ccccc8c8ccccc8c7c6)ccc5c34)n2)cc1]

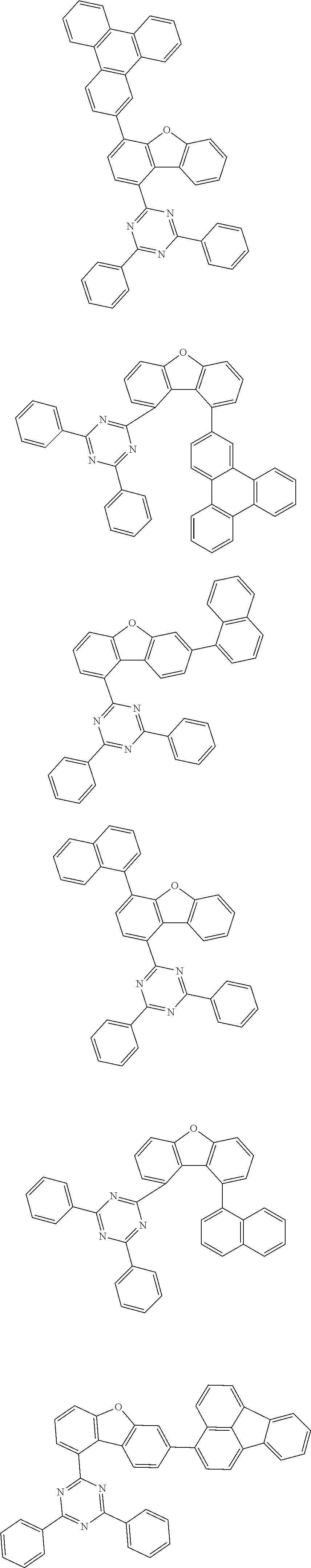

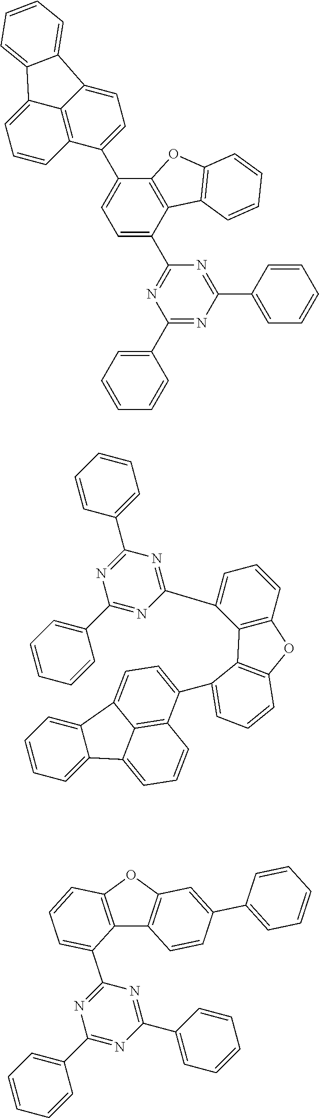 [SMILES: c1ccc(-c2nc(-c3ccccc3)nc(-c3ccc(-c4ccc5c6c(cccc46)-c4ccccc4-5)c4oc5ccccc5c34)n2)cc1 ; c1ccc(-c2nc(-c3ccccc3)nc(-c3cccc4oc5cccc(-c6ccc7c8c(cccc68)-c6ccccc6-7)c5c34)n2)cc1 ; c1ccc(-c2ccc3c(c2)oc2cccc(-c4nc(-c5ccccc5)nc(-c5ccccc5)n4)c23)cc1]

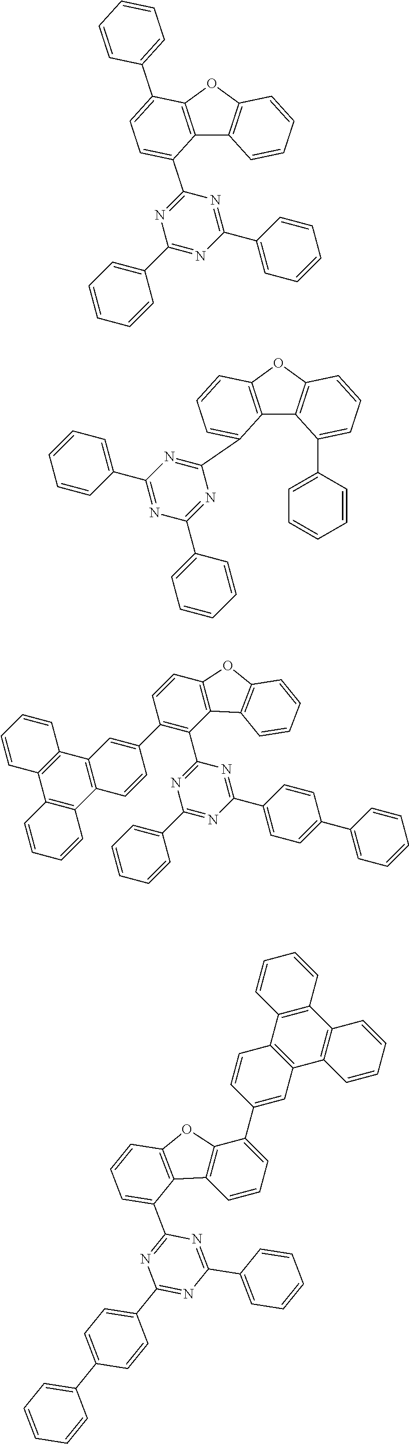 [SMILES: c1ccc(-c2nc(-c3ccccc3)nc(-c3ccc(-c4ccccc4)c4oc5ccccc5c34)n2)cc1 ; c1ccc(-c2nc(-c3ccccc3)nc(-c3cccc4oc5cccc(-c6ccccc6)c5c34)n2)cc1 ; c1ccc(-c2ccc(-c3nc(-c4ccccc4)nc(-c4c(-c5ccc6c7ccccc7c7ccccc7c6c5)ccc5oc6ccccc6c45)n3)cc2)cc1 ; c1ccc(-c2ccc(-c3nc(-c4ccccc4)nc(-c4cccc5oc6c(-c7ccc8c9ccccc9c9ccccc9c8c7)cccc6c45)n3)cc2)cc1]

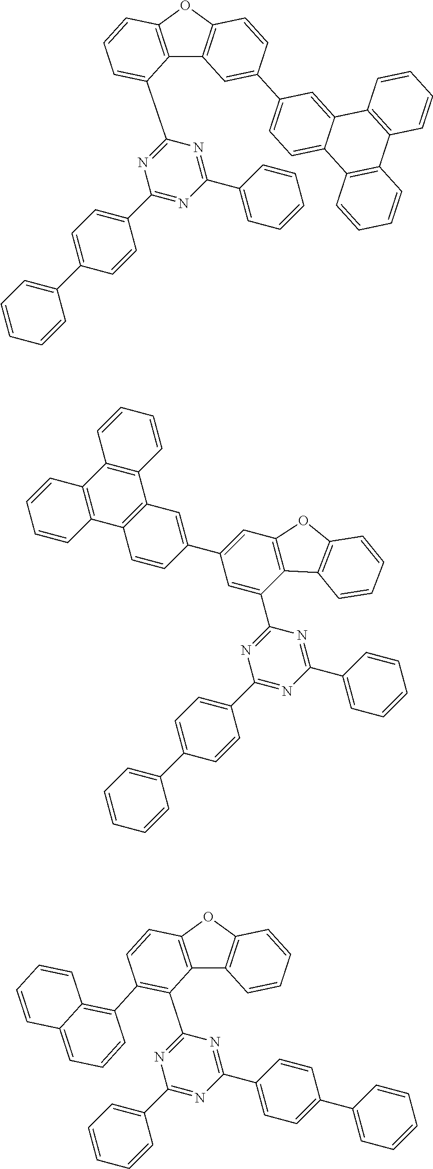 [SMILES: c1ccc(-c2ccc(-c3nc(-c4ccccc4)nc(-c4cccc5oc6ccc(-c7ccc8c9ccccc9c9ccccc9c8c7)cc6c45)n3)cc2)cc1 ; c1ccc(-c2ccc(-c3nc(-c4ccccc4)nc(-c4cc(-c5ccc6c7ccccc7c7ccccc7c6c5)cc5oc6ccccc6c45)n3)cc2)cc1 ; c1ccc(-c2ccc(-c3nc(-c4ccccc4)nc(-c4c(-c5cccc6ccccc56)ccc5oc6ccccc6c45)n3)cc2)cc1]

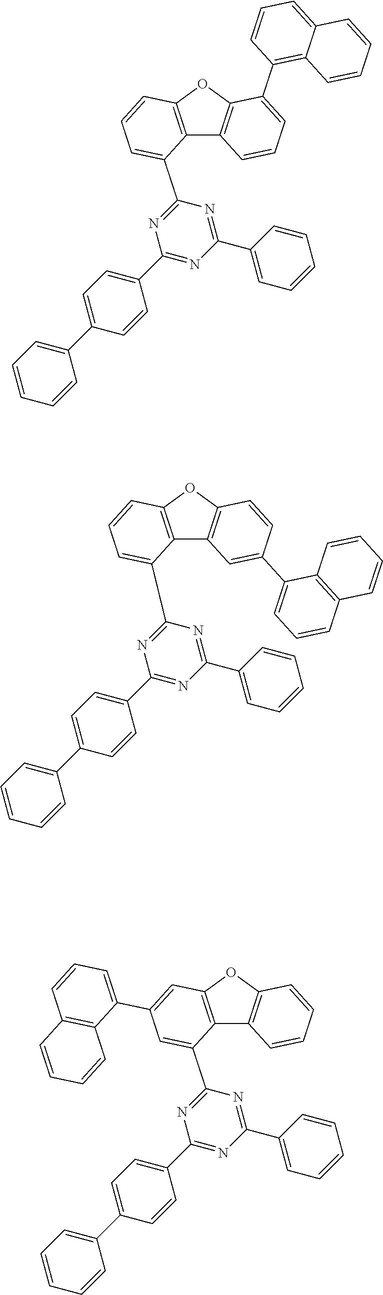 [SMILES: c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1 ; c1ccc(-c2ccc(-c3nc(-c4ccccc4)nc(-c4cc(-c5cccc6ccccc56)cc5oc6ccccc6c45)n3)cc2)cc1]

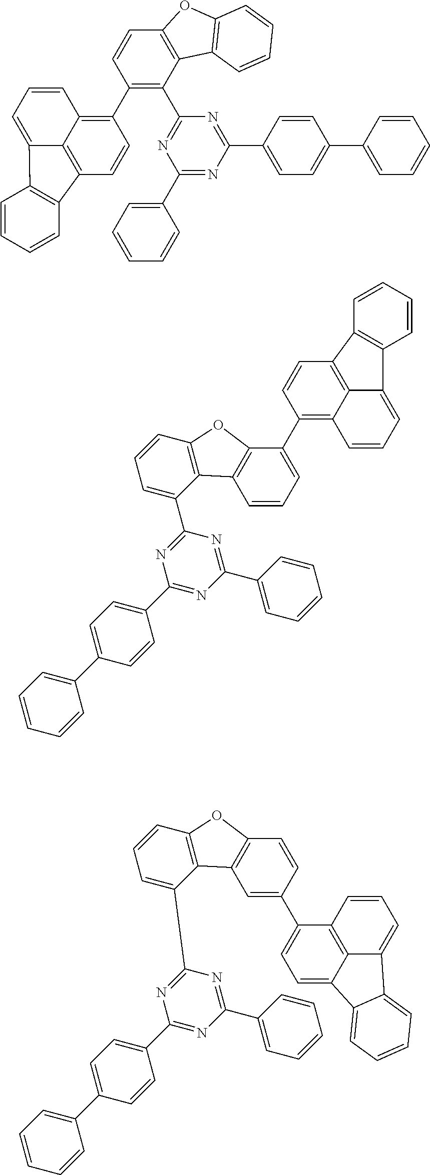 [SMILES: c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1 ; c1ccc(-c2ccc(-c3nc(-c4ccccc4)nc(-c4cccc5oc6ccc(-c7ccc8c9c(cccc79)-c7ccccc7-8)cc6c45)n3)cc2)cc1]

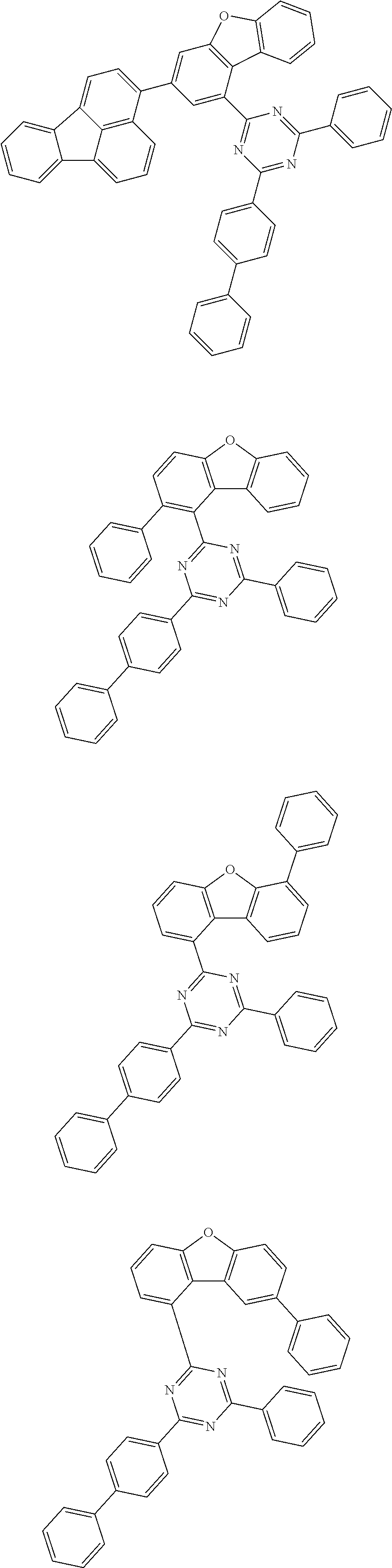 [SMILES: c1ccc(-c2ccc(-c3nc(-c4ccccc4)nc(-c4cc(-c5ccc6c7c(cccc57)-c5ccccc5-6)cc5oc6ccccc6c45)n3)cc2)cc1 ; c1ccc(-c2ccc(-c3nc(-c4ccccc4)nc(-c4c(-c5ccccc5)ccc5oc6ccccc6c45)n3)cc2)cc1 ; c1ccc(-c2ccc(-c3nc(-c4ccccc4)nc(-c4cccc5oc6c(-c7ccccc7)cccc6c45)n3)cc2)cc1 ; c1ccc(-c2ccc(-c3nc(-c4ccccc4)nc(-c4cccc5oc6ccc(-c7ccccc7)cc6c45)n3)cc2)cc1]

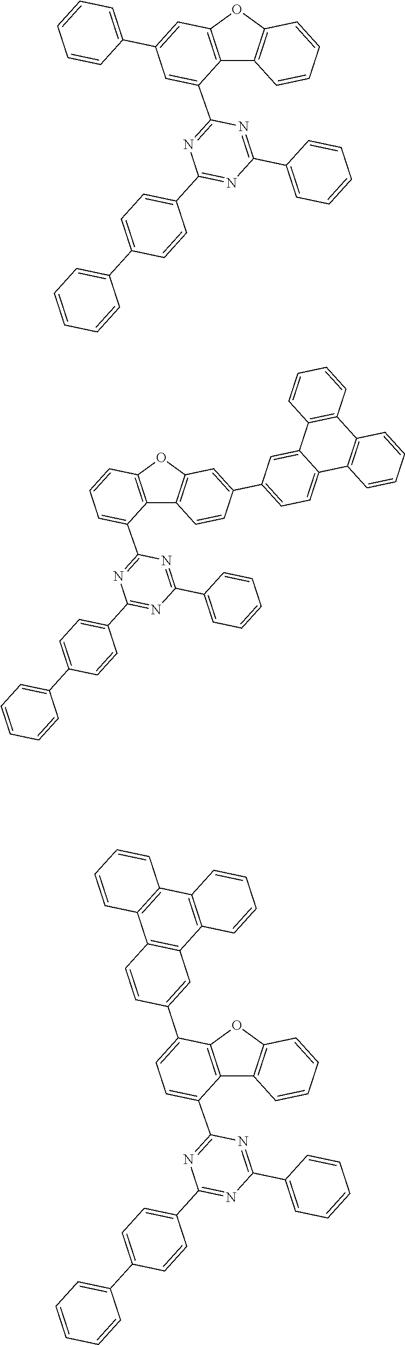 [SMILES: c1ccc(-c2ccc(-c3nc(-c4ccccc4)nc(-c4cc(-c5ccccc5)cc5oc6ccccc6c45)n3)cc2)cc1 ; c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4ccc(-c5ccc6c7ccccc7c7ccccc7c6c5)c5oc6ccccc6c45)n3)cc2)cc1]

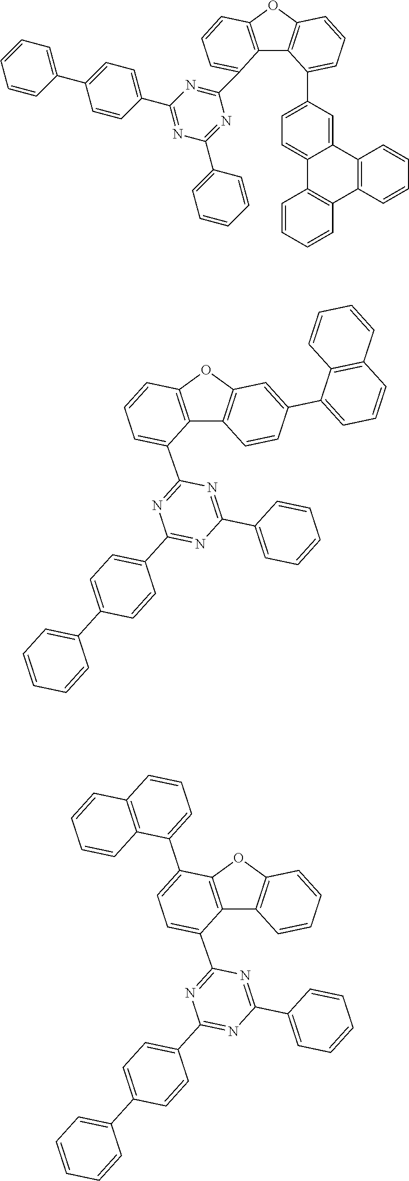 [SMILES: c1ccc(-c2ccc(-c3nc(-c4ccccc4)nc(-c4cccc5oc6cccc(-c7ccc8c9ccccc9c9ccccc9c8c7)c6c45)n3)cc2)cc1 ; c1ccc(-c2ccc(-c3nc(-c4ccccc4)nc(-c4cccc5oc6cc(-c7cccc8ccccc78)ccc6c45)n3)cc2)cc1 ; c1ccc(-c2ccc(-c3nc(-c4ccccc4)nc(-c4ccc(-c5cccc6ccccc56)c5oc6ccccc6c45)n3)cc2)cc1]

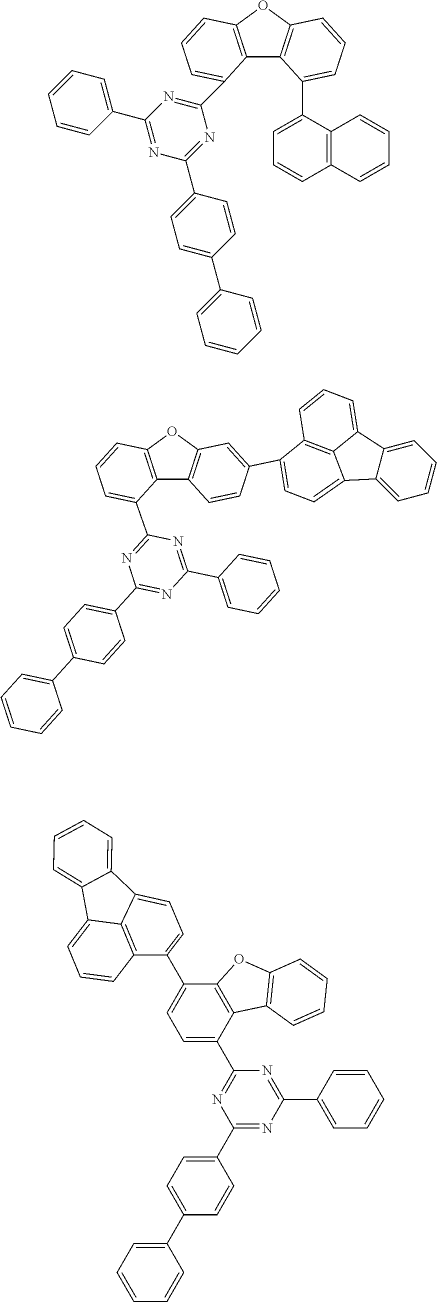 [SMILES: c1ccc(-c2ccc(-c3nc(-c4ccccc4)nc(-c4cccc5oc6cccc(-c7cccc8ccccc78)c6c45)n3)cc2)cc1 ; c1ccc(-c2ccc(-c3nc(-c4ccccc4)nc(-c4cccc5oc6cc(-c7ccc8c9c(cccc79)-c7ccccc7-8)ccc6c45)n3)cc2)cc1 ; c1ccc(-c2ccc(-c3nc(-c4ccccc4)nc(-c4ccc(-c5ccc6c7c(cccc57)-c5ccccc5-6)c5oc6ccccc6c45)n3)cc2)cc1]

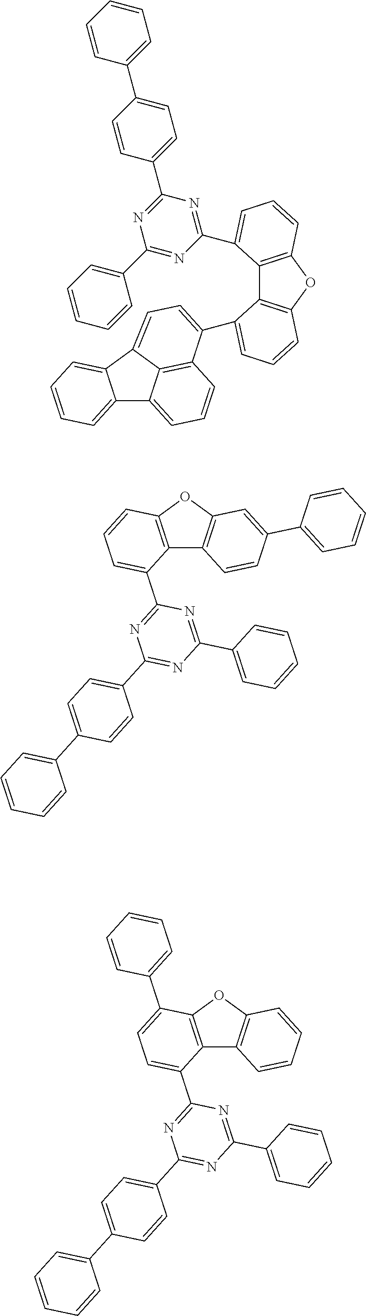 [SMILES: c1ccc(-c2ccc(-c3nc(-c4ccccc4)nc(-c4cccc5oc6cccc(-c7ccc8c9c(cccc79)-c7ccccc7-8)c6c45)n3)cc2)cc1 ; c1ccc(-c2ccc(-c3nc(-c4ccccc4)nc(-c4cccc5oc6cc(-c7ccccc7)ccc6c45)n3)cc2)cc1 ; c1ccc(-c2ccc(-c3nc(-c4ccccc4)nc(-c4ccc(-c5ccccc5)c5oc6ccccc6c45)n3)cc2)cc1]

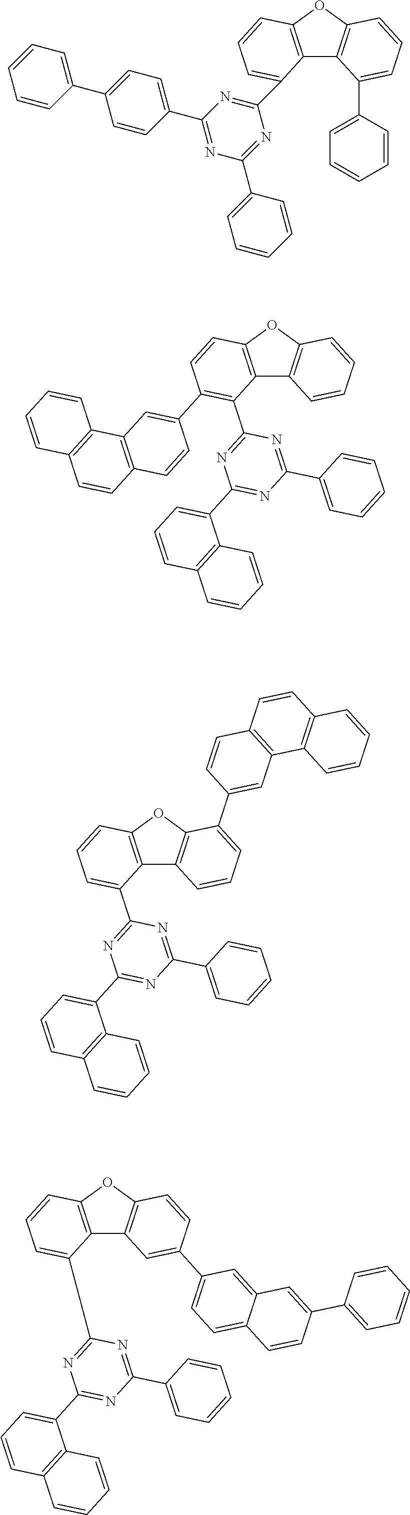 [SMILES: c1ccc(-c2ccc(-c3nc(-c4ccccc4)nc(-c4cccc5oc6cccc(-c7ccccc7)c6c45)n3)cc2)cc1 ; c1ccc(-c2nc(-c3cccc4ccccc34)nc(-c3c(-c4ccc5ccc6ccccc6c5c4)ccc4oc5ccccc5c34)n2)cc1 ; c1ccc(-c2nc(-c3cccc4ccccc34)nc(-c3cccc4oc5c(-c6ccc7ccc8ccccc8c7c6)cccc5c34)n2)cc1 ; c1ccc(-c2ccc3ccc(-c4ccc5oc6cccc(-c7nc(-c8ccccc8)nc(-c8cccc9ccccc89)n7)c6c5c4)cc3c2)cc1]

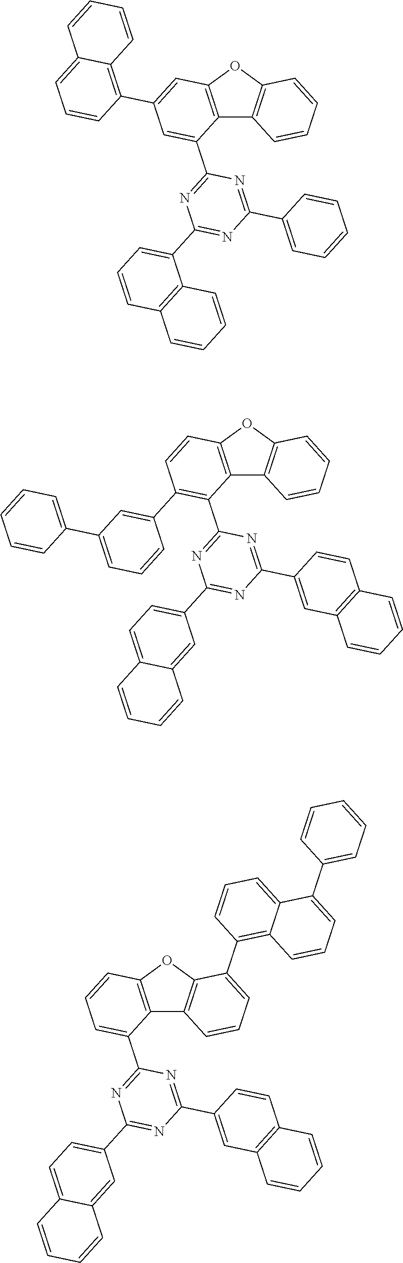 [SMILES: c1ccc(-c2nc(-c3cccc4ccccc34)nc(-c3cc(-c4cccc5ccccc45)cc4oc5ccccc5c34)n2)cc1 ; c1ccc(-c2cccc(-c3ccc4oc5ccccc5c4c3-c3nc(-c4ccc5ccccc5c4)nc(-c4ccc5ccccc5c4)n3)c2)cc1 ; c1ccc(-c2cccc3c(-c4cccc5c4oc4cccc(-c6nc(-c7ccc8ccccc8c7)nc(-c7ccc8ccccc8c7)n6)c45)cccc23)cc1]

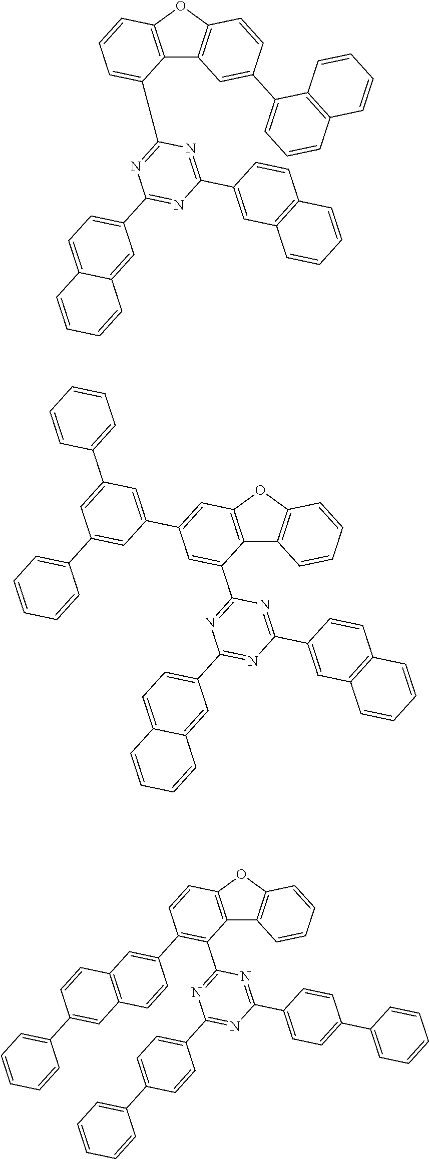 [SMILES: c1ccc2cc(-c3nc(-c4ccc5ccccc5c4)nc(-c4cccc5oc6ccc(-c7cccc8ccccc78)cc6c45)n3)ccc2c1 ; c1ccc(-c2cc(-c3ccccc3)cc(-c3cc(-c4nc(-c5ccc6ccccc6c5)nc(-c5ccc6ccccc6c5)n4)c4c(c3)oc3ccccc34)c2)cc1 ; c1ccc(-c2ccc(-c3nc(-c4ccc(-c5ccccc5)cc4)nc(-c4c(-c5ccc6cc(-c7ccccc7)ccc6c5)ccc5oc6ccccc6c45)n3)cc2)cc1]

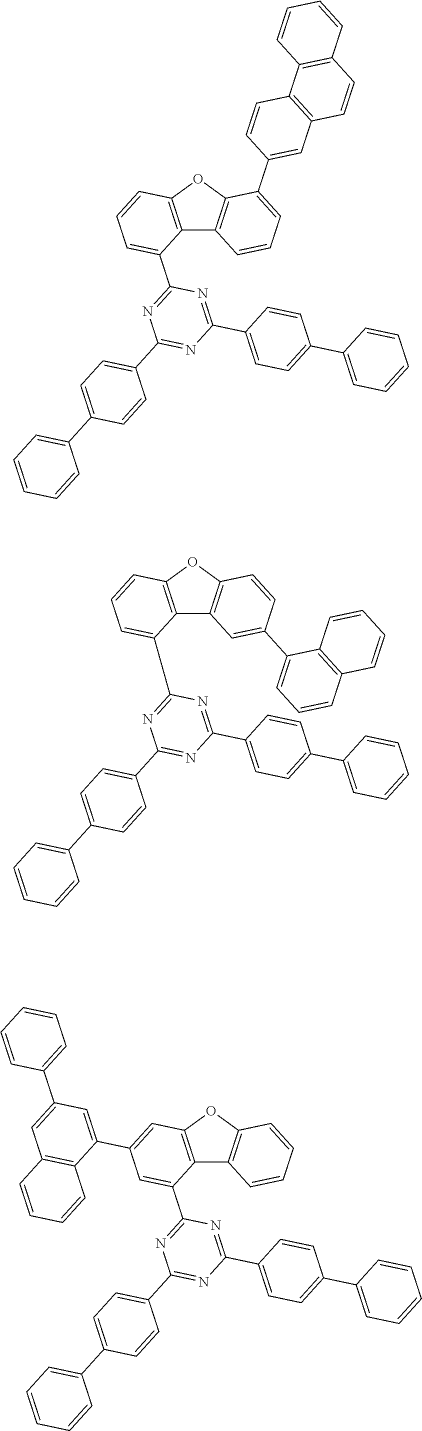 [SMILES: c1ccc(-c2ccc(-c3nc(-c4ccc(-c5ccccc5)cc4)nc(-c4cccc5oc6c(-c7ccc8c(ccc9ccccc98)c7)cccc6c45)n3)cc2)cc1 ; c1ccc(-c2ccc(-c3nc(-c4ccc(-c5ccccc5)cc4)nc(-c4cccc5oc6ccc(-c7cccc8ccccc78)cc6c45)n3)cc2)cc1 ; c1ccc(-c2ccc(-c3nc(-c4ccc(-c5ccccc5)cc4)nc(-c4cc(-c5cc(-c6ccccc6)cc6ccccc56)cc5oc6ccccc6c45)n3)cc2)cc1]

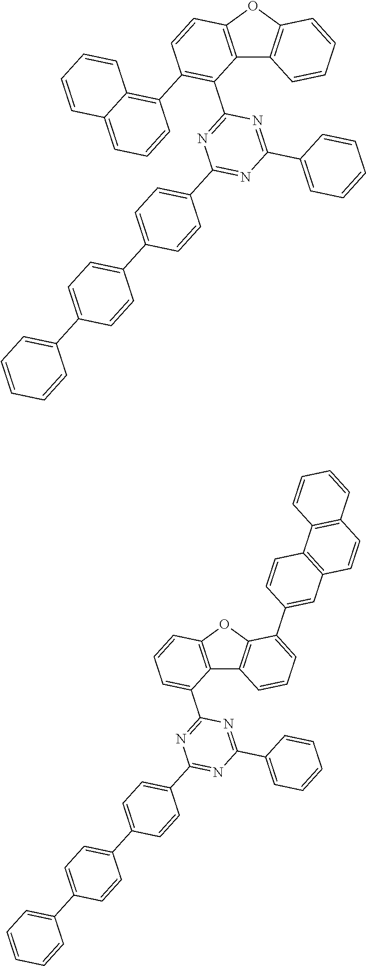 [SMILES: c1ccc(-c2ccc(-c3ccc(-c4nc(-c5ccccc5)nc(-c5c(-c6cccc7ccccc67)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1]

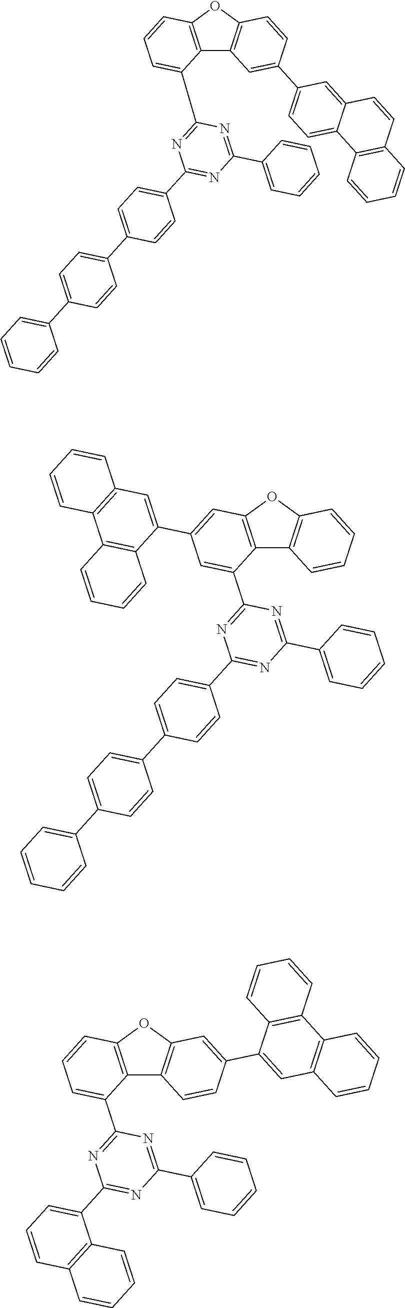 [SMILES: c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1 ; c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2nc(-c3cccc4ccccc34)nc(-c3cccc4oc5cc(-c6cc7ccccc7c7ccccc67)ccc5c34)n2)cc1]

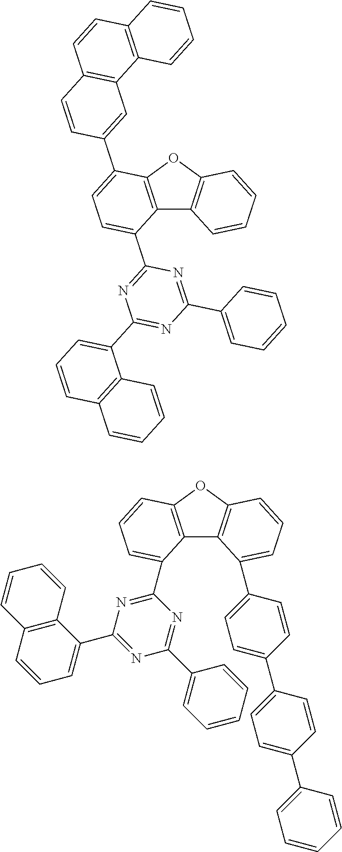 [SMILES: c1ccc(-c2nc(-c3cccc4ccccc34)nc(-c3ccc(-c4ccc5ccc6ccccc6c5c4)c4oc5ccccc5c34)n2)cc1 ; c1ccc(-c2ccc(-c3ccc(-c4cccc5oc6cccc(-c7nc(-c8ccccc8)nc(-c8cccc9ccccc89)n7)c6c45)cc3)cc2)cc1]

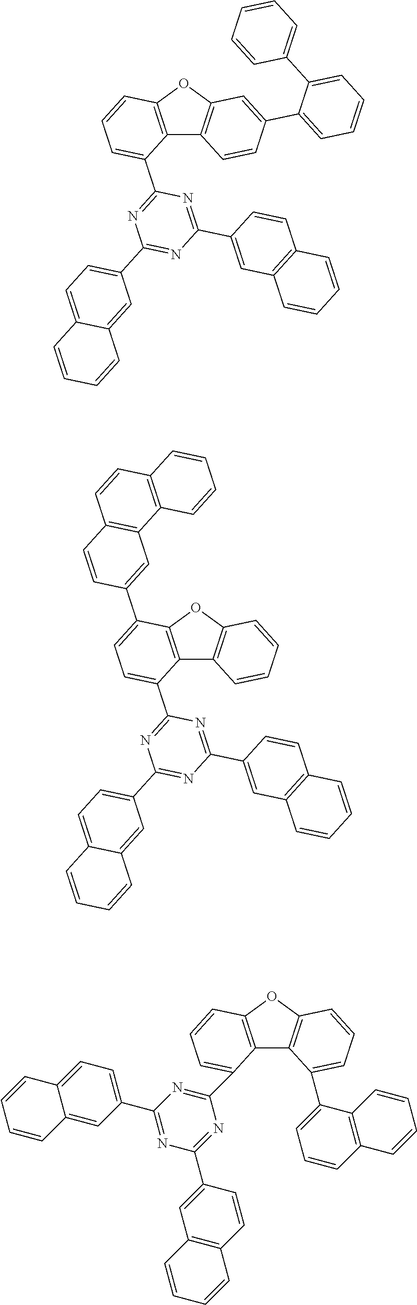 [SMILES: c1ccc(-c2ccccc2-c2ccc3c(c2)oc2cccc(-c4nc(-c5ccc6ccccc6c5)nc(-c5ccc6ccccc6c5)n4)c23)cc1 ; c1ccc2cc(-c3nc(-c4ccc5ccccc5c4)nc(-c4ccc(-c5ccc6ccc7ccccc7c6c5)c5oc6ccccc6c45)n3)ccc2c1 ; c1ccc2cc(-c3nc(-c4ccc5ccccc5c4)nc(-c4cccc5oc6cccc(-c7cccc8ccccc78)c6c45)n3)ccc2c1]

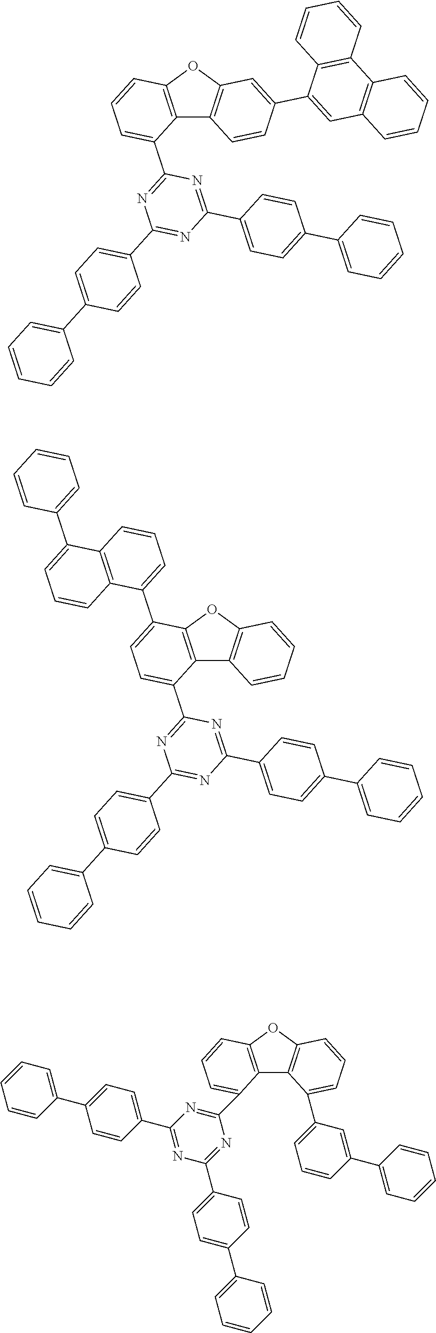 [SMILES: c1ccc(-c2ccc(-c3nc(-c4ccc(-c5ccccc5)cc4)nc(-c4cccc5oc6cc(-c7cc8ccccc8c8ccccc78)ccc6c45)n3)cc2)cc1 ; c1ccc(-c2ccc(-c3nc(-c4ccc(-c5ccccc5)cc4)nc(-c4ccc(-c5cccc6c(-c7ccccc7)cccc56)c5oc6ccccc6c45)n3)cc2)cc1 ; c1ccc(-c2ccc(-c3nc(-c4ccc(-c5ccccc5)cc4)nc(-c4cccc5oc6cccc(-c7cccc(-c8ccccc8)c7)c6c45)n3)cc2)cc1]

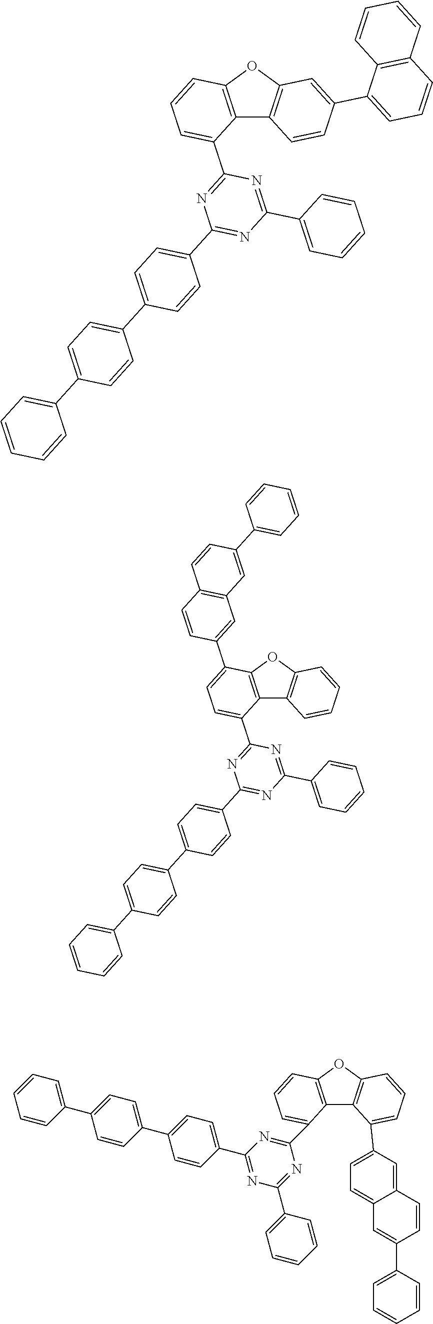 [SMILES: c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1]

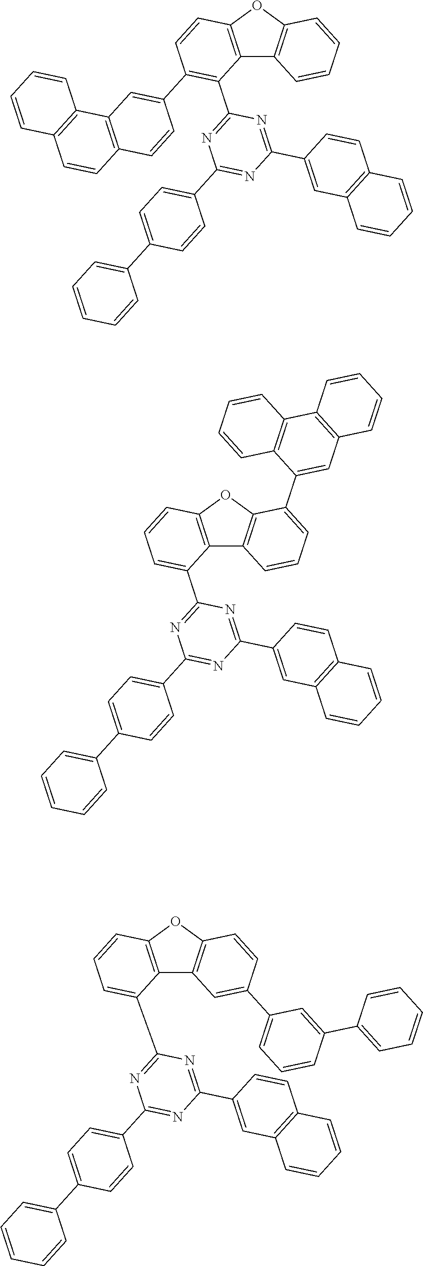 [SMILES: c1ccc(-c2ccc(-c3nc(-c4ccc5ccccc5c4)nc(-c4c(-c5ccc6ccc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8ccccc8c8ccccc78)cccc6c45)n3)cc2)cc1 ; c1ccc(-c2ccc(-c3nc(-c4ccc5ccccc5c4)nc(-c4cccc5oc6ccc(-c7cccc(-c8ccccc8)c7)cc6c45)n3)cc2)cc1]

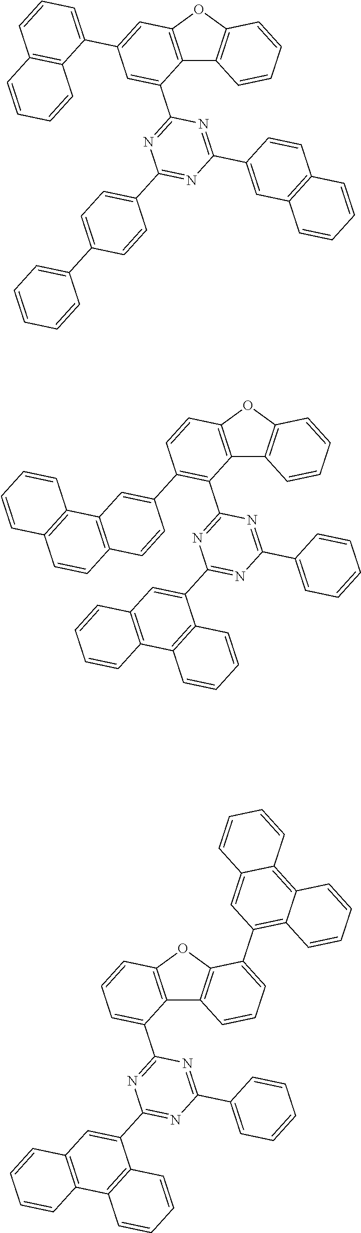 [SMILES: c1ccc(-c2ccc(-c3nc(-c4ccc5ccccc5c4)nc(-c4cc(-c5cccc6ccccc56)cc5oc6ccccc6c45)n3)cc2)cc1 ; c1ccc(-c2nc(-c3cc4ccccc4c4ccccc34)nc(-c3c(-c4ccc5ccc6ccccc6c5c4)ccc4oc5ccccc5c34)n2)cc1 ; c1ccc(-c2nc(-c3cc4ccccc4c4ccccc34)nc(-c3cccc4oc5c(-c6cc7ccccc7c7ccccc67)cccc5c34)n2)cc1]

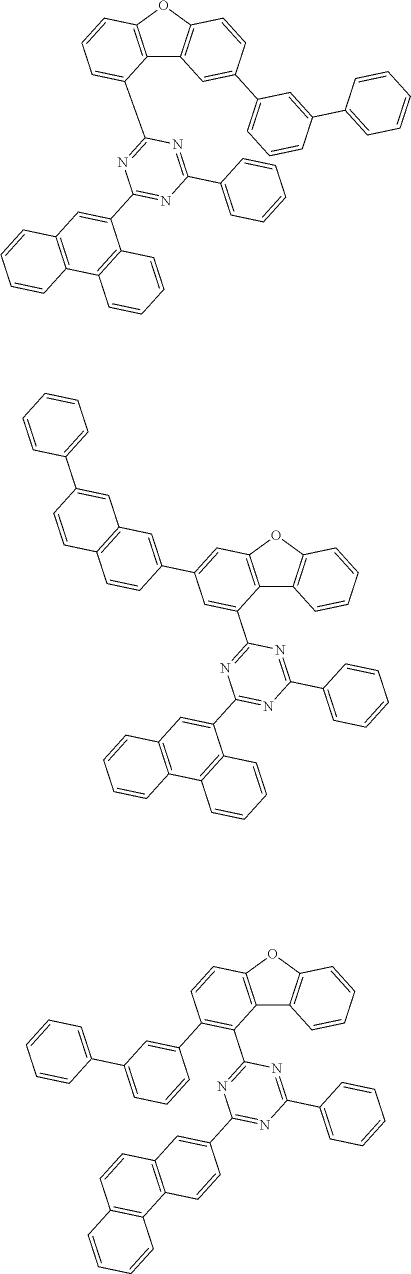 [SMILES: c1ccc(-c2cccc(-c3ccc4oc5cccc(-c6nc(-c7ccccc7)nc(-c7cc8ccccc8c8ccccc78)n6)c5c4c3)c2)cc1 ; c1ccc(-c2ccc3ccc(-c4cc(-c5nc(-c6ccccc6)nc(-c6cc7ccccc7c7ccccc67)n5)c5c(c4)oc4ccccc45)cc3c2)cc1 ; c1ccc(-c2cccc(-c3ccc4oc5ccccc5c4c3-c3nc(-c4ccccc4)nc(-c4ccc5c(ccc6ccccc65)c4)n3)c2)cc1]

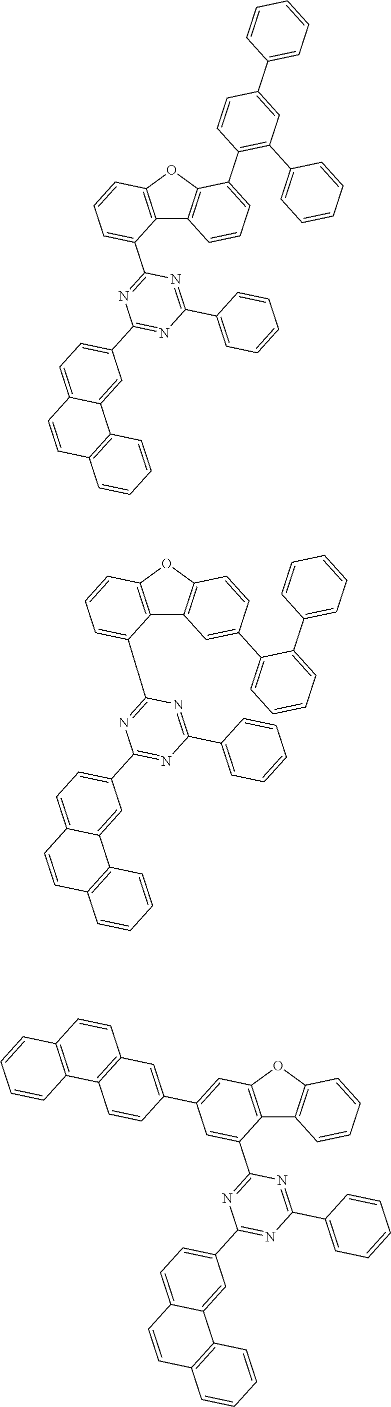 [SMILES: c1ccc(-c2ccc(-c3cccc4c3oc3cccc(-c5nc(-c6ccccc6)nc(-c6ccc7ccc8ccccc8c7c6)n5)c34)c(-c3ccccc3)c2)cc1 ; c1ccc(-c2nc(-c3ccc4ccc5ccccc5c4c3)nc(-c3cccc4oc5ccc(-c6ccccc6-c6ccccc6)cc5c34)n2)cc1 ; c1ccc(-c2nc(-c3ccc4ccc5ccccc5c4c3)nc(-c3cc(-c4ccc5c(ccc6ccccc65)c4)cc4oc5ccccc5c34)n2)cc1]

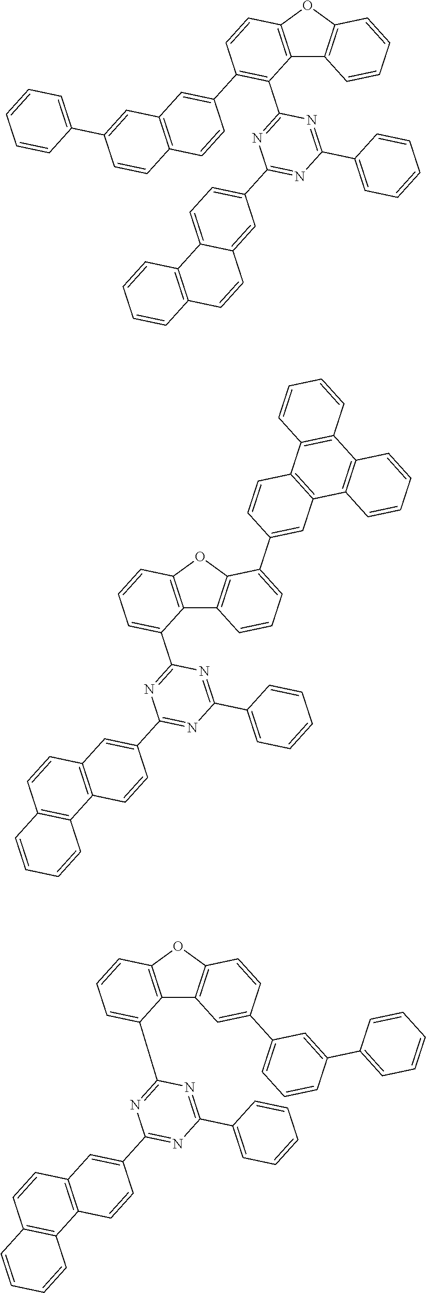 [SMILES: c1ccc(-c2ccc3ccc(-c4ccc5oc6ccccc6c5c4-c4nc(-c5ccccc5)nc(-c5ccc6c(ccc7ccccc76)c5)n4)cc3c2)cc1 ; c1ccc(-c2nc(-c3ccc4c(ccc5ccccc54)c3)nc(-c3cccc4oc5c(-c6ccc7c8ccccc8c8ccccc8c7c6)cccc5c34)n2)cc1 ; c1ccc(-c2cccc(-c3ccc4oc5cccc(-c6nc(-c7ccccc7)nc(-c7ccc8c(ccc9ccccc98)c7)n6)c5c4c3)c2)cc1]

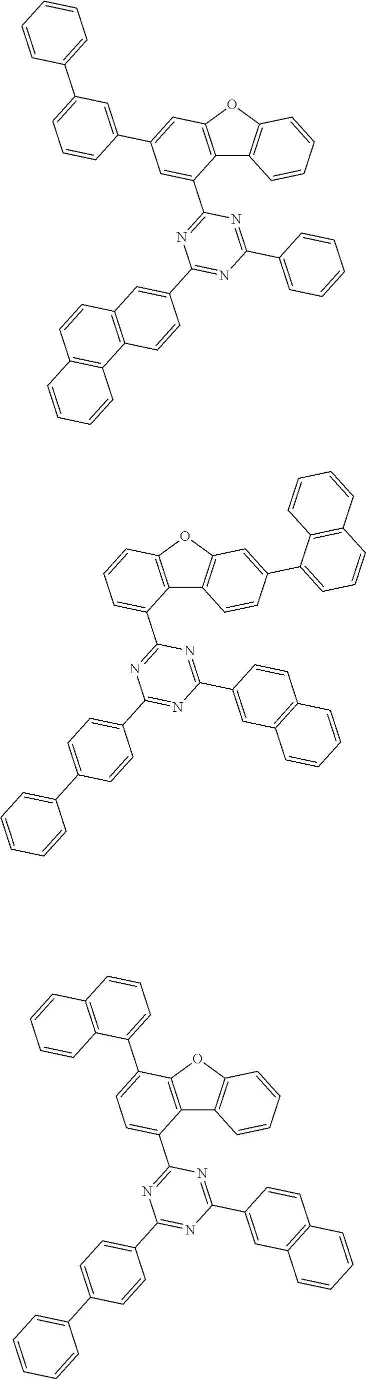 [SMILES: c1ccc(-c2cccc(-c3cc(-c4nc(-c5ccccc5)nc(-c5ccc6c(ccc7ccccc76)c5)n4)c4c(c3)oc3ccccc34)c2)cc1 ; c1ccc(-c2ccc(-c3nc(-c4ccc5ccccc5c4)nc(-c4cccc5oc6cc(-c7cccc8ccccc78)ccc6c45)n3)cc2)cc1 ; c1ccc(-c2ccc(-c3nc(-c4ccc5ccccc5c4)nc(-c4ccc(-c5cccc6ccccc56)c5oc6ccccc6c45)n3)cc2)cc1]

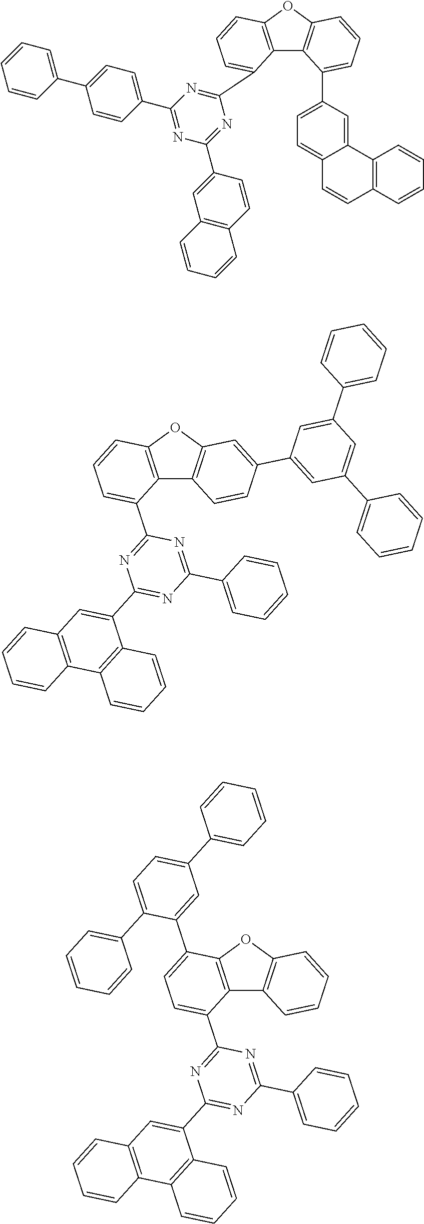 [SMILES: c1ccc(-c2ccc(-c3nc(-c4ccc5ccccc5c4)nc(-c4cccc5oc6cccc(-c7ccc8ccc9ccccc9c8c7)c6c45)n3)cc2)cc1 ; c1ccc(-c2cc(-c3ccccc3)cc(-c3ccc4c(c3)oc3cccc(-c5nc(-c6ccccc6)nc(-c6cc7ccccc7c7ccccc67)n5)c34)c2)cc1 ; c1ccc(-c2ccc(-c3ccccc3)c(-c3ccc(-c4nc(-c5ccccc5)nc(-c5cc6ccccc6c6ccccc56)n4)c4c3oc3ccccc34)c2)cc1]

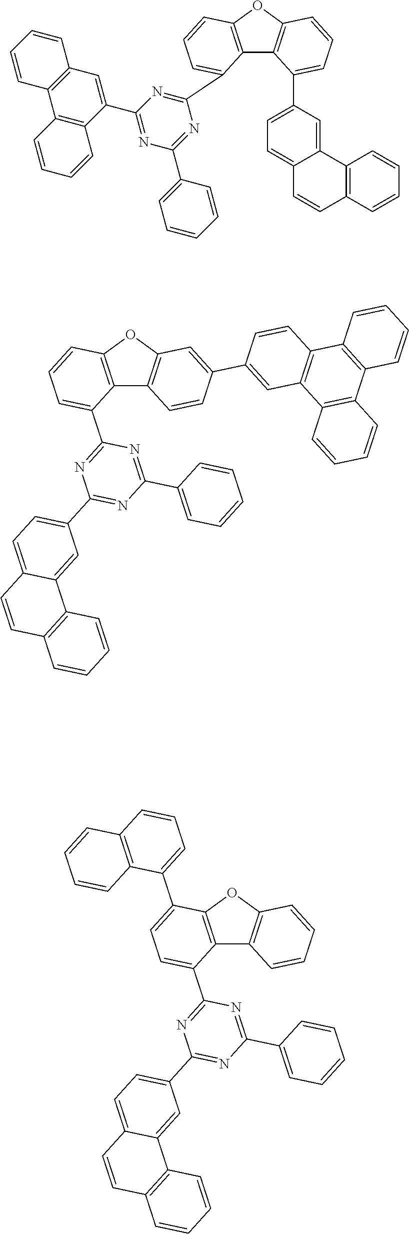 [SMILES: c1ccc(-c2nc(-c3cc4ccccc4c4ccccc34)nc(-c3cccc4oc5cccc(-c6ccc7ccc8ccccc8c7c6)c5c34)n2)cc1 ; c1ccc(-c2nc(-c3ccc4ccc5ccccc5c4c3)nc(-c3cccc4oc5cc(-c6ccc7c8ccccc8c8ccccc8c7c6)ccc5c34)n2)cc1 ; c1ccc(-c2nc(-c3ccc4ccc5ccccc5c4c3)nc(-c3ccc(-c4cccc5ccccc45)c4oc5ccccc5c34)n2)cc1]

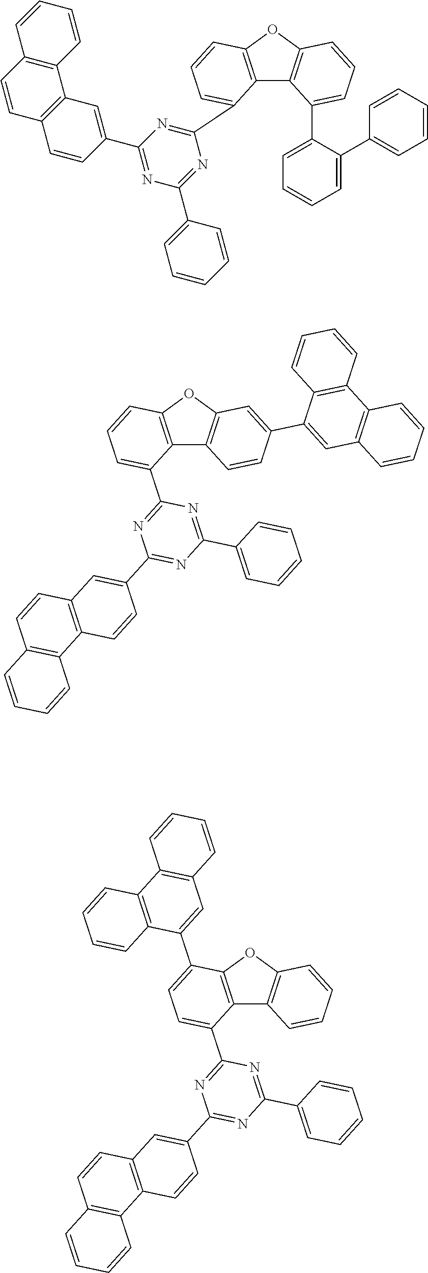 [SMILES: c1ccc(-c2nc(-c3ccc4ccc5ccccc5c4c3)nc(-c3cccc4oc5cccc(-c6ccccc6-c6ccccc6)c5c34)n2)cc1 ; c1ccc(-c2nc(-c3ccc4c(ccc5ccccc54)c3)nc(-c3cccc4oc5cc(-c6cc7ccccc7c7ccccc67)ccc5c34)n2)cc1 ; c1ccc(-c2nc(-c3ccc4c(ccc5ccccc54)c3)nc(-c3ccc(-c4cc5ccccc5c5ccccc45)c4oc5ccccc5c34)n2)cc1]

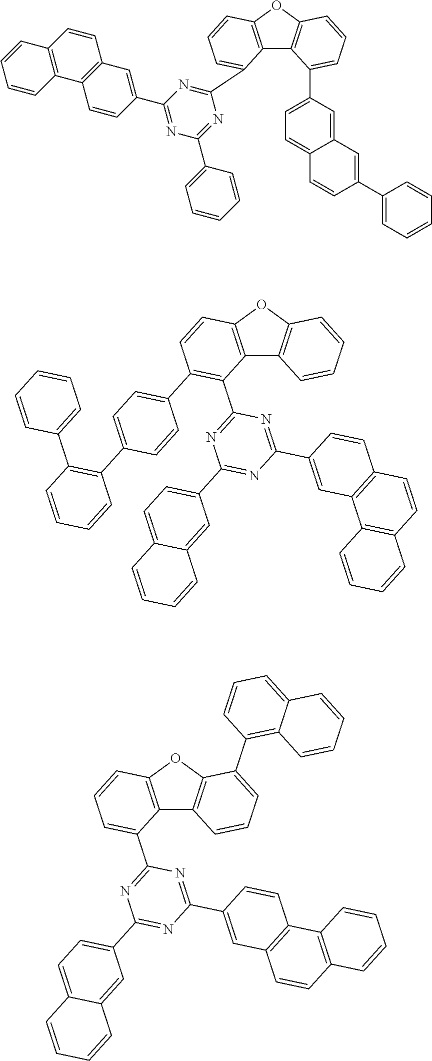 [SMILES: c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1 ; c1ccc2cc(-c3nc(-c4ccc5c(ccc6ccccc65)c4)nc(-c4cccc5oc6c(-c7cccc8ccccc78)cccc6c45)n3)ccc2c1]

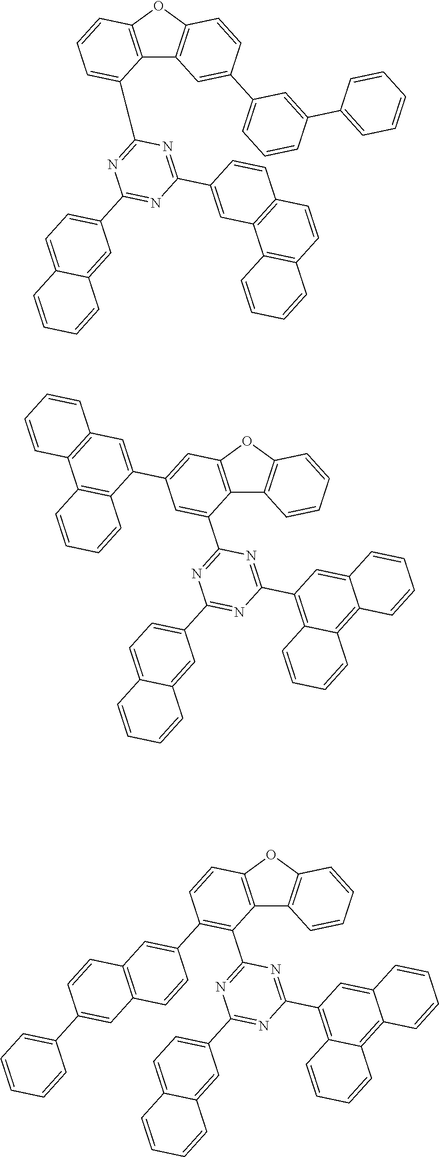 [SMILES: c1ccc(-c2cccc(-c3ccc4oc5cccc(-c6nc(-c7ccc8ccccc8c7)nc(-c7ccc8ccc9ccccc9c8c7)n6)c5c4c3)c2)cc1 ; c1ccc2cc(-c3nc(-c4cc5ccccc5c5ccccc45)nc(-c4cc(-c5cc6ccccc6c6ccccc56)cc5oc6ccccc6c45)n3)ccc2c1 ; c1ccc(-c2ccc3cc(-c4ccc5oc6ccccc6c5c4-c4nc(-c5ccc6ccccc6c5)nc(-c5cc6ccccc6c6ccccc56)n4)ccc3c2)cc1]

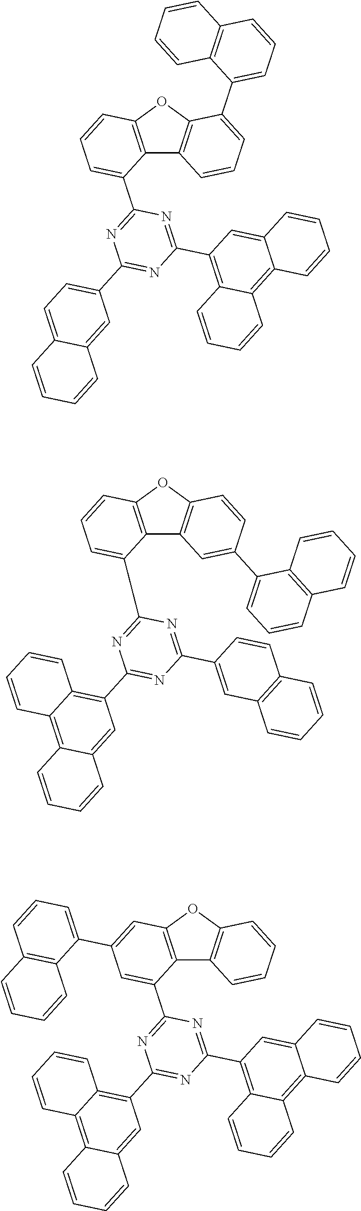 [SMILES: c1ccc2cc(-c3nc(-c4cc5ccccc5c5ccccc45)nc(-c4cccc5oc6c(-c7cccc8ccccc78)cccc6c45)n3)ccc2c1 ; c1ccc2cc(-c3nc(-c4cc5ccccc5c5ccccc45)nc(-c4cccc5oc6ccc(-c7cccc8ccccc78)cc6c45)n3)ccc2c1 ; c1ccc2c(-c3cc(-c4nc(-c5cc6ccccc6c6ccccc56)nc(-c5cc6ccccc6c6ccccc56)n4)c4c(c3)oc3ccccc34)cccc2c1]

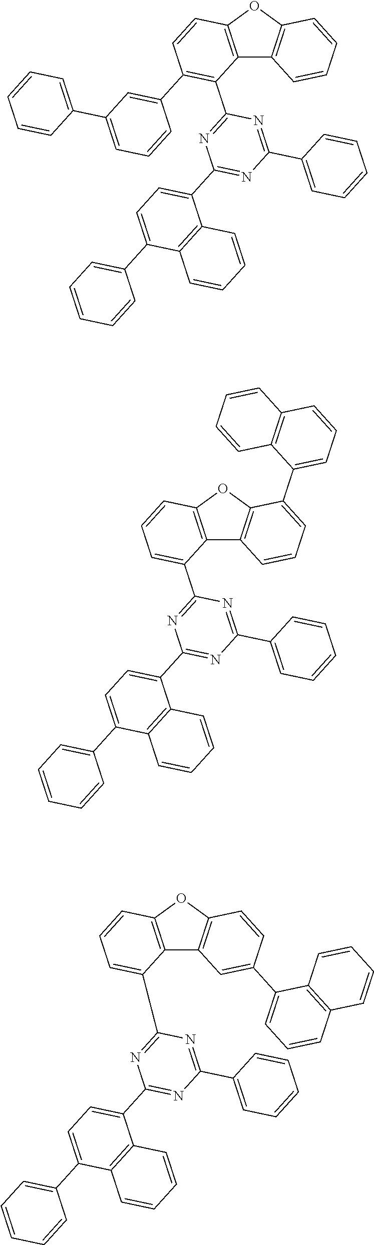 [SMILES: c1ccc(-c2cccc(-c3ccc4oc5ccccc5c4c3-c3nc(-c4ccccc4)nc(-c4ccc(-c5ccccc5)c5ccccc45)n3)c2)cc1 ; c1ccc(-c2nc(-c3ccc(-c4ccccc4)c4ccccc34)nc(-c3cccc4oc5c(-c6cccc7ccccc67)cccc5c34)n2)cc1 ; c1ccc(-c2nc(-c3ccc(-c4ccccc4)c4ccccc34)nc(-c3cccc4oc5ccc(-c6cccc7ccccc67)cc5c34)n2)cc1]

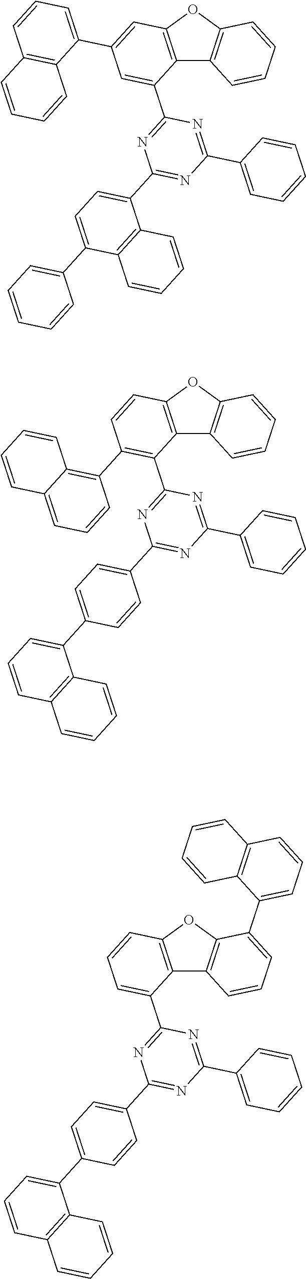 [SMILES: c1ccc(-c2nc(-c3ccc(-c4ccccc4)c4ccccc34)nc(-c3cc(-c4cccc5ccccc45)cc4oc5ccccc5c34)n2)cc1 ; c1ccc(-c2nc(-c3ccc(-c4cccc5ccccc45)cc3)nc(-c3c(-c4cccc5ccccc45)ccc4oc5ccccc5c34)n2)cc1 ; c1ccc(-c2nc(-c3ccc(-c4cccc5ccccc45)cc3)nc(-c3cccc4oc5c(-c6cccc7ccccc67)cccc5c34)n2)cc1]

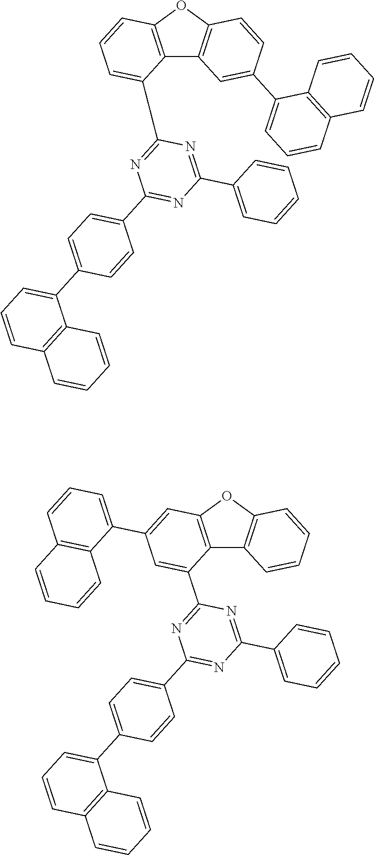 [SMILES: c1ccc(-c2nc(-c3ccc(-c4cccc5ccccc45)cc3)nc(-c3cccc4oc5ccc(-c6cccc7ccccc67)cc5c34)n2)cc1 ; c1ccc(-c2nc(-c3ccc(-c4cccc5ccccc45)cc3)nc(-c3cc(-c4cccc5ccccc45)cc4oc5ccccc5c34)n2)cc1]

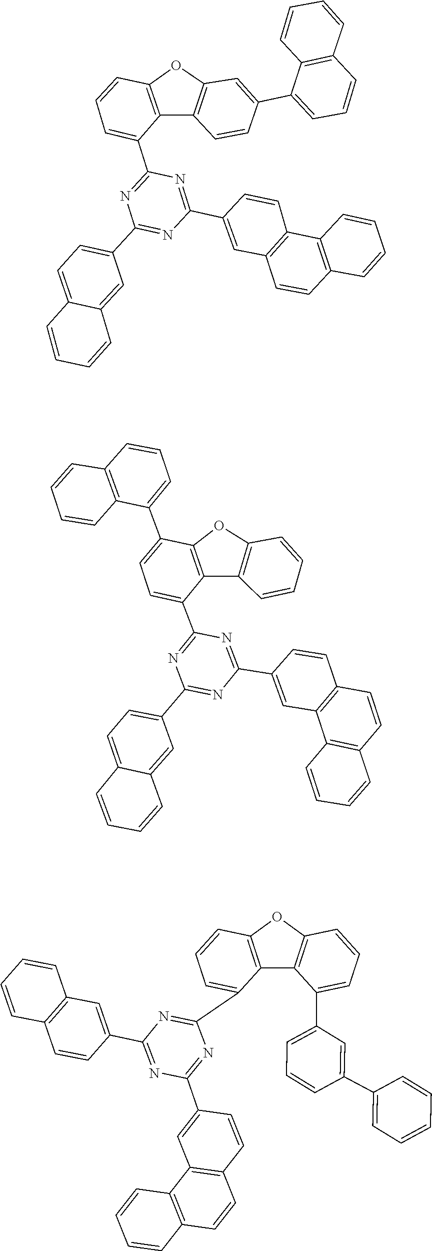 [SMILES: c1ccc2cc(-c3nc(-c4ccc5c(ccc6ccccc65)c4)nc(-c4cccc5oc6cc(-c7cccc8ccccc78)ccc6c45)n3)ccc2c1 ; c1ccc2cc(-c3nc(-c4ccc5ccc6ccccc6c5c4)nc(-c4ccc(-c5cccc6ccccc56)c5oc6ccccc6c45)n3)ccc2c1 ; c1ccc(-c2cccc(-c3cccc4oc5cccc(-c6nc(-c7ccc8ccccc8c7)nc(-c7ccc8ccc9ccccc9c8c7)n6)c5c34)c2)cc1]

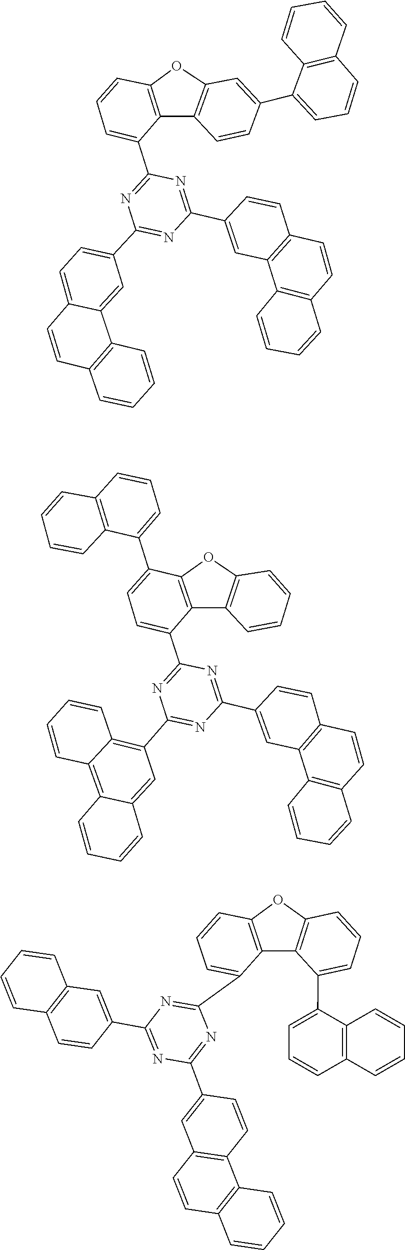 [SMILES: c1ccc2c(-c3ccc4c(c3)oc3cccc(-c5nc(-c6ccc7ccc8ccccc8c7c6)nc(-c6ccc7ccc8ccccc8c7c6)n5)c34)cccc2c1 ; c1ccc2c(-c3ccc(-c4nc(-c5ccc6ccc7ccccc7c6c5)nc(-c5cc6ccccc6c6ccccc56)n4)c4c3oc3ccccc34)cccc2c1 ; c1ccc2cc(-c3nc(-c4ccc5c(ccc6ccccc65)c4)nc(-c4cccc5oc6cccc(-c7cccc8ccccc78)c6c45)n3)ccc2c1]

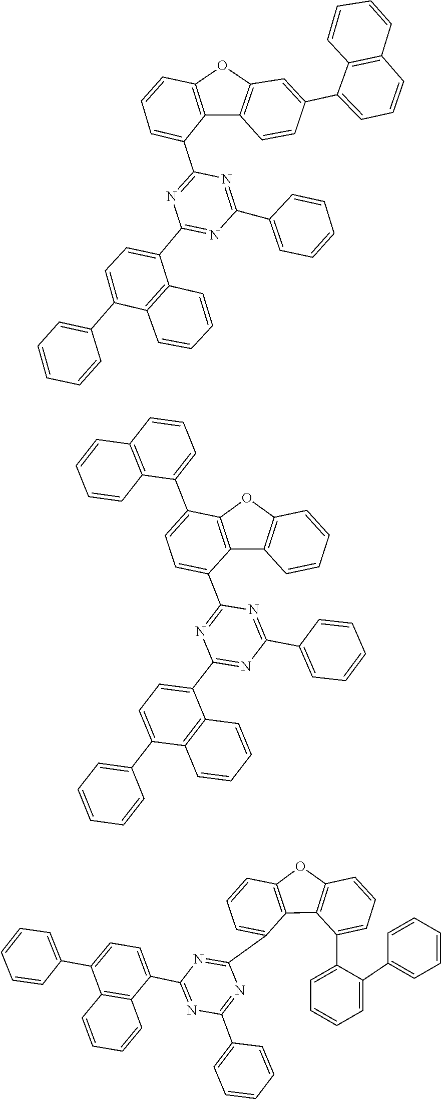 [SMILES: c1ccc(-c2nc(-c3ccc(-c4ccccc4)c4ccccc34)nc(-c3cccc4oc5cc(-c6cccc7ccccc67)ccc5c34)n2)cc1 ; c1ccc(-c2nc(-c3ccc(-c4ccccc4)c4ccccc34)nc(-c3ccc(-c4cccc5ccccc45)c4oc5ccccc5c34)n2)cc1 ; c1ccc(-c2nc(-c3ccc(-c4ccccc4)c4ccccc34)nc(-c3cccc4oc5cccc(-c6ccccc6-c6ccccc6)c5c34)n2)cc1]

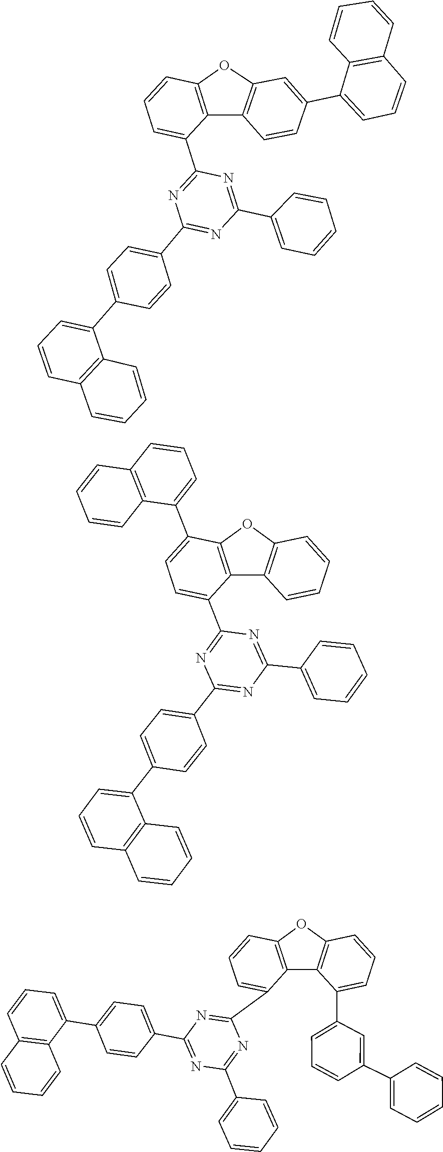

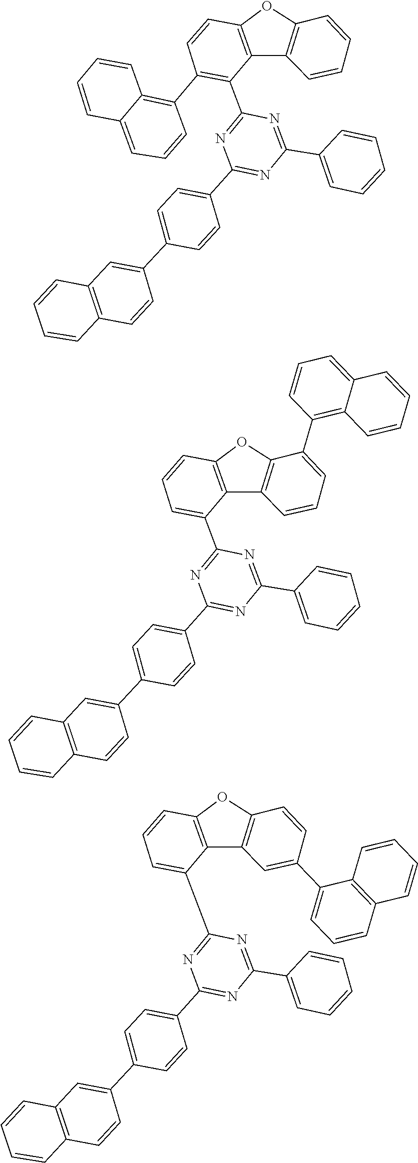 [SMILES: c1ccc(-c2nc(-c3ccc(-c4ccc5ccccc5c4)cc3)nc(-c3c(-c4cccc5ccccc45)ccc4oc5ccccc5c34)n2)cc1 ; c1ccc(-c2nc(-c3ccc(-c4ccc5ccccc5c4)cc3)nc(-c3cccc4oc5c(-c6cccc7ccccc67)cccc5c34)n2)cc1 ; c1ccc(-c2nc(-c3ccc(-c4ccc5ccccc5c4)cc3)nc(-c3cccc4oc5ccc(-c6cccc7ccccc67)cc5c34)n2)cc1]

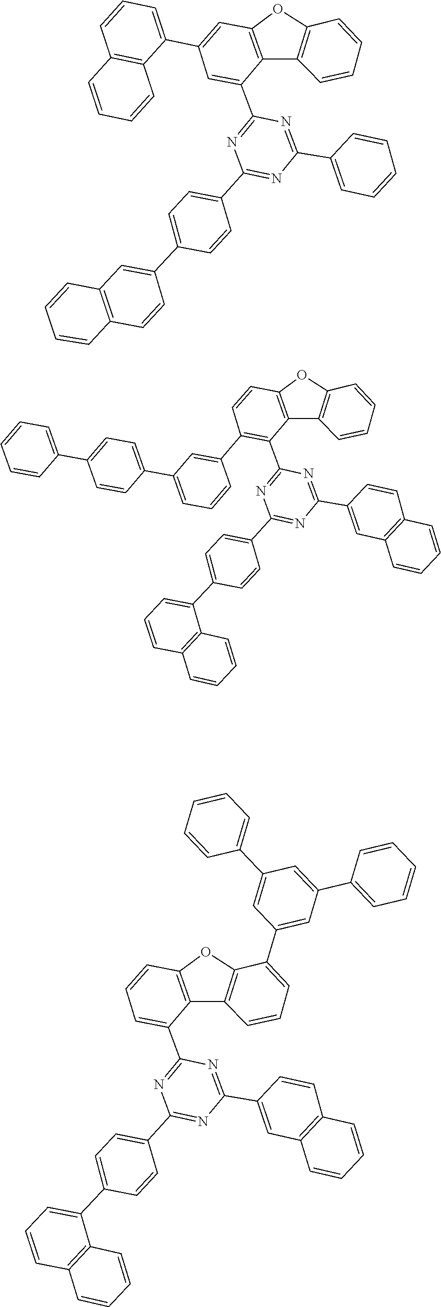 [SMILES: c1ccc(-c2nc(-c3ccc(-c4ccc5ccccc5c4)cc3)nc(-c3cc(-c4cccc5ccccc45)cc4oc5ccccc5c34)n2)cc1 ; c1ccc(-c2ccc(-c3cccc(-c4ccc5oc6ccccc6c5c4-c4nc(-c5ccc(-c6cccc7ccccc67)cc5)nc(-c5ccc6ccccc6c5)n4)c3)cc2)cc1 ; c1ccc(-c2cc(-c3ccccc3)cc(-c3cccc4c3oc3cccc(-c5nc(-c6ccc(-c7cccc8ccccc78)cc6)nc(-c6ccc7ccccc7c6)n5)c34)c2)cc1]

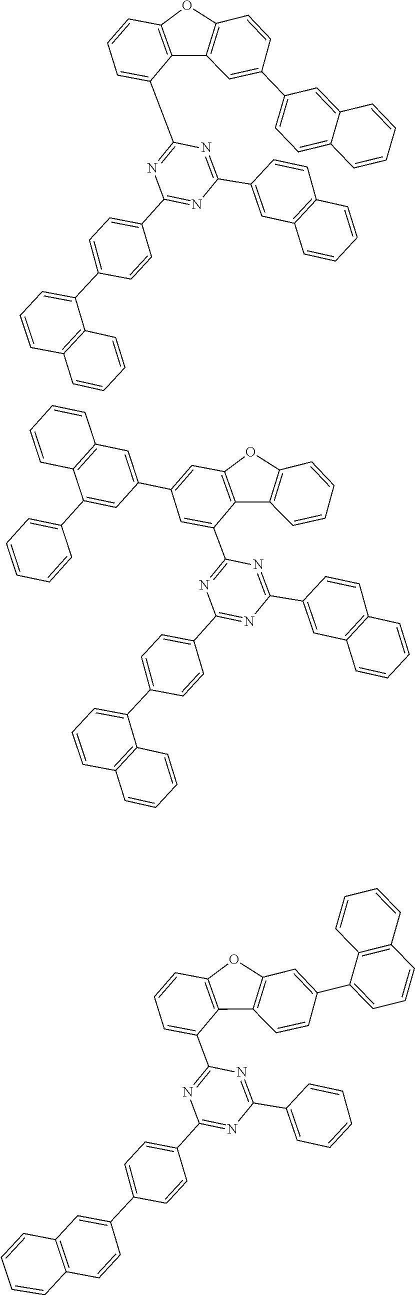

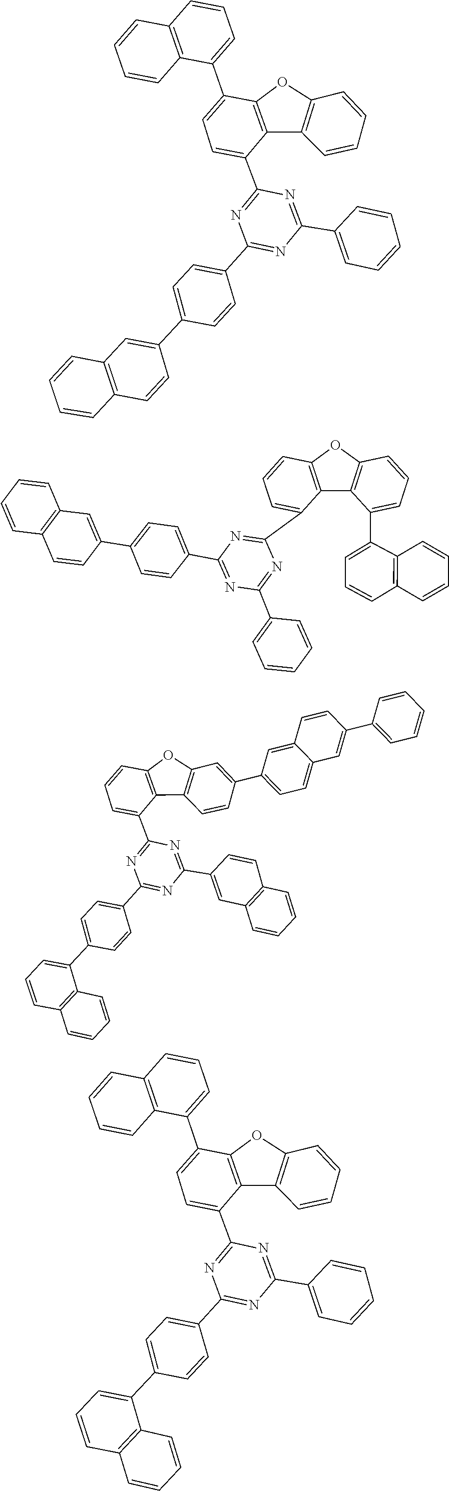

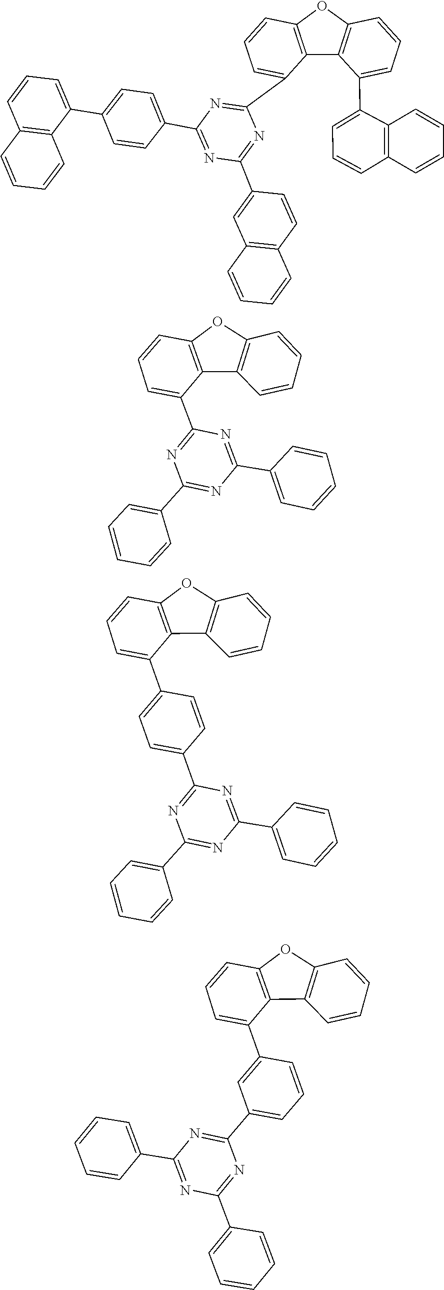

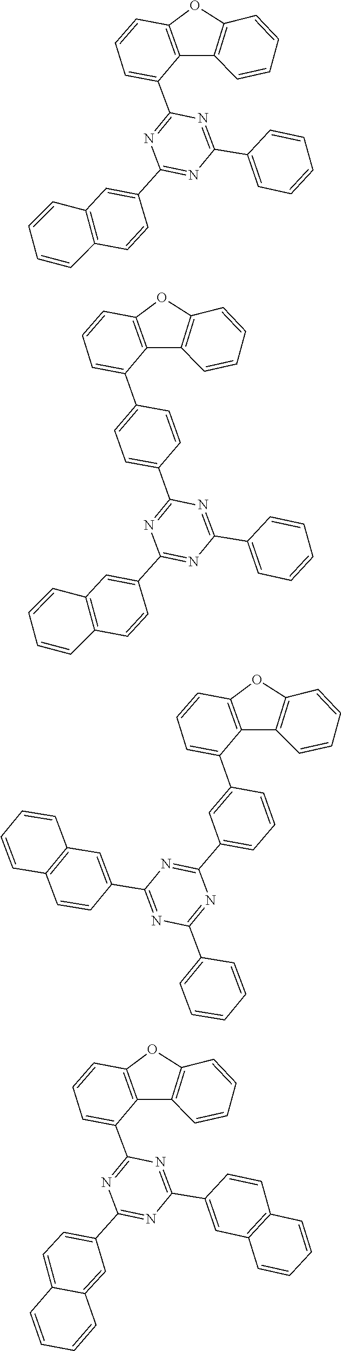

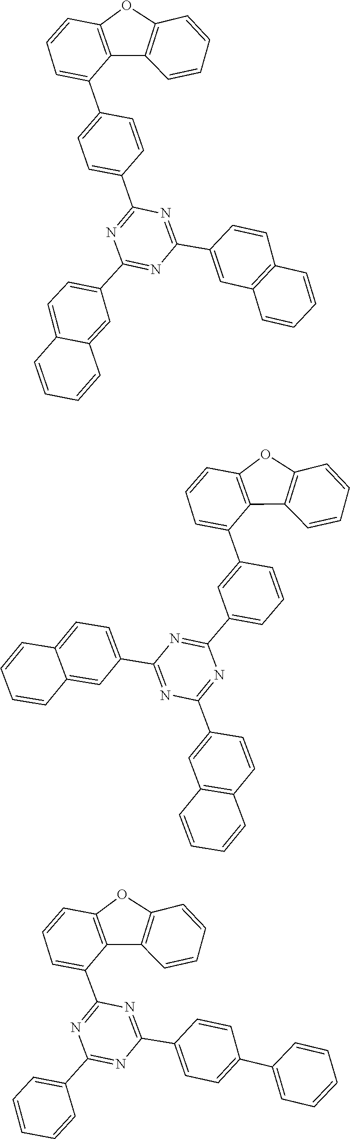 [SMILES: c1ccc2cc(-c3nc(-c4ccc(-c5cccc6oc7ccccc7c56)cc4)nc(-c4ccc5ccccc5c4)n3)ccc2c1 ; c1cc(-c2nc(-c3ccc4ccccc4c3)nc(-c3ccc4ccccc4c3)n2)cc(-c2cccc3oc4ccccc4c23)c1 ; c1ccc(-c2ccc(-c3nc(-c4ccccc4)nc(-c4cccc5oc6ccccc6c45)n3)cc2)cc1]

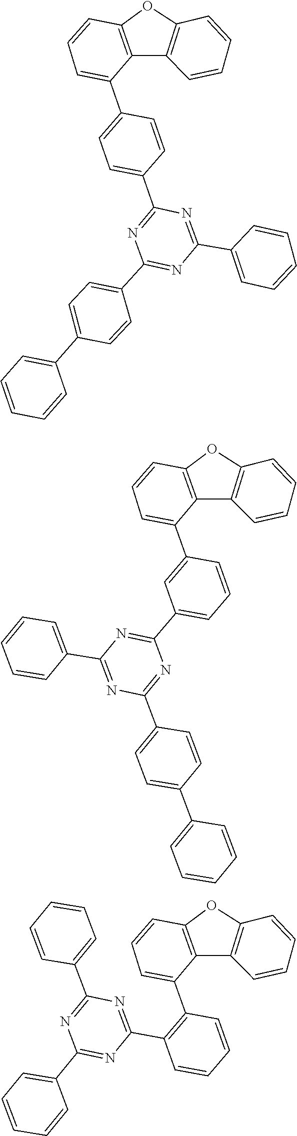

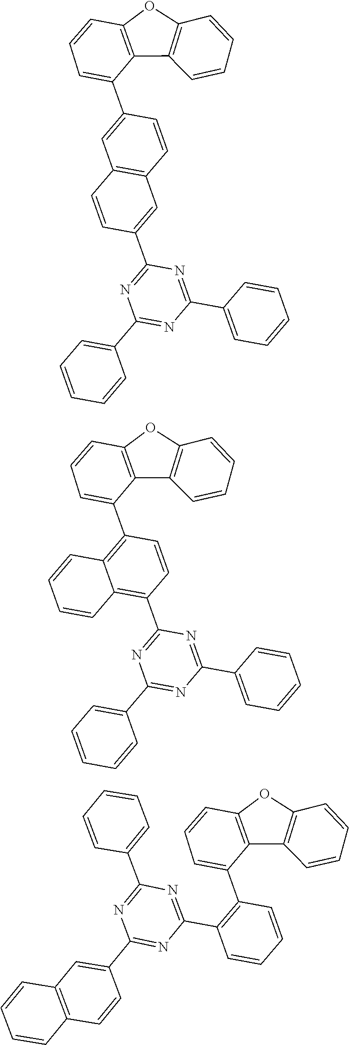

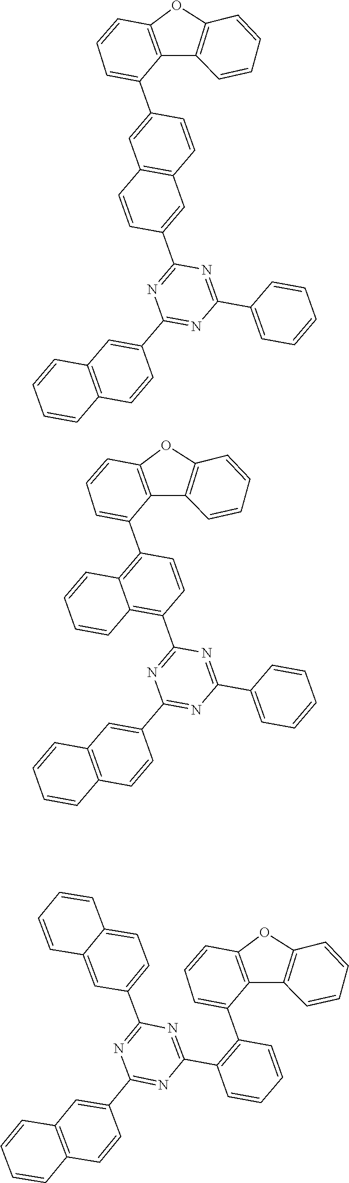 [SMILES: c1ccc(-c2nc(-c3ccc4ccccc4c3)nc(-c3ccc4cc(-c5cccc6oc7ccccc7c56)ccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2cccc3oc4ccccc4c23)c(-c2nc(-c3ccc4ccccc4c3)nc(-c3ccc4ccccc4c3)n2)c1]

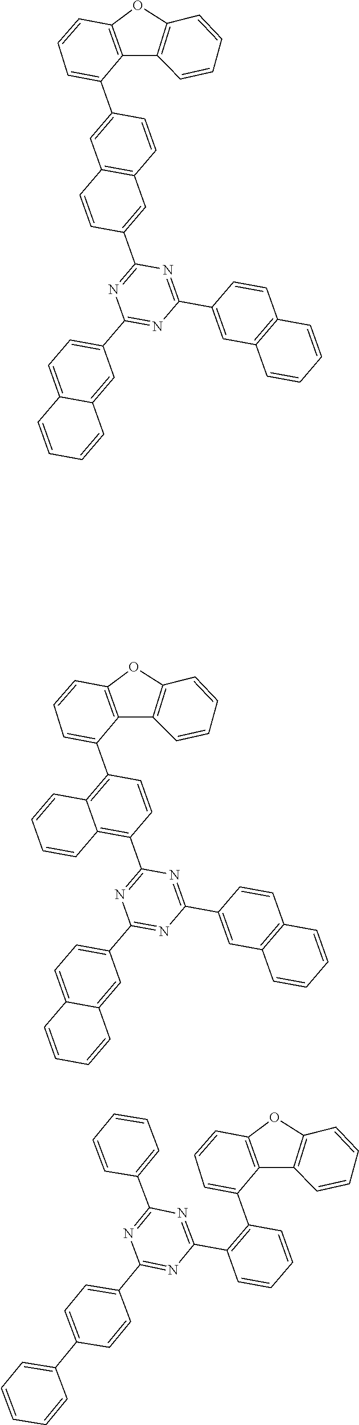 [SMILES: c1ccc2cc(-c3nc(-c4ccc5ccccc5c4)nc(-c4ccc5cc(-c6cccc7oc8ccccc8c67)ccc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2ccc(-c3nc(-c4ccccc4)nc(-c4ccccc4-c4cccc5oc6ccccc6c45)n3)cc2)cc1]

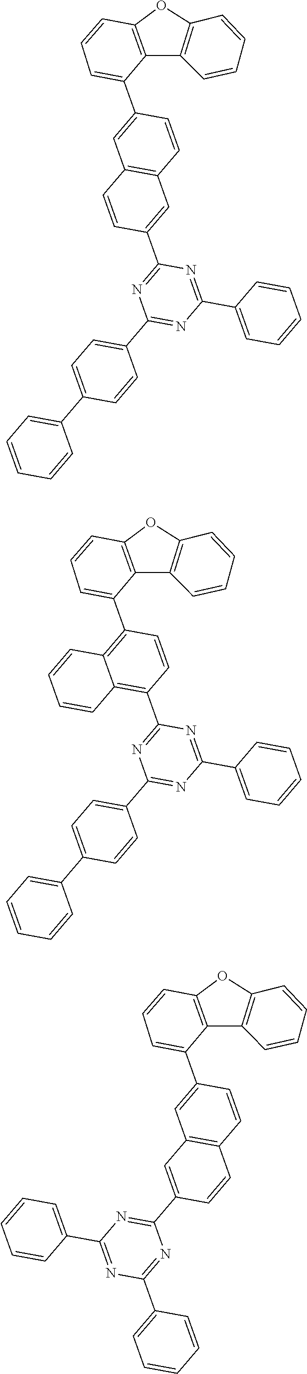 [SMILES: c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2nc(-c3ccccc3)nc(-c3ccc4ccc(-c5cccc6oc7ccccc7c56)cc4c3)n2)cc1]

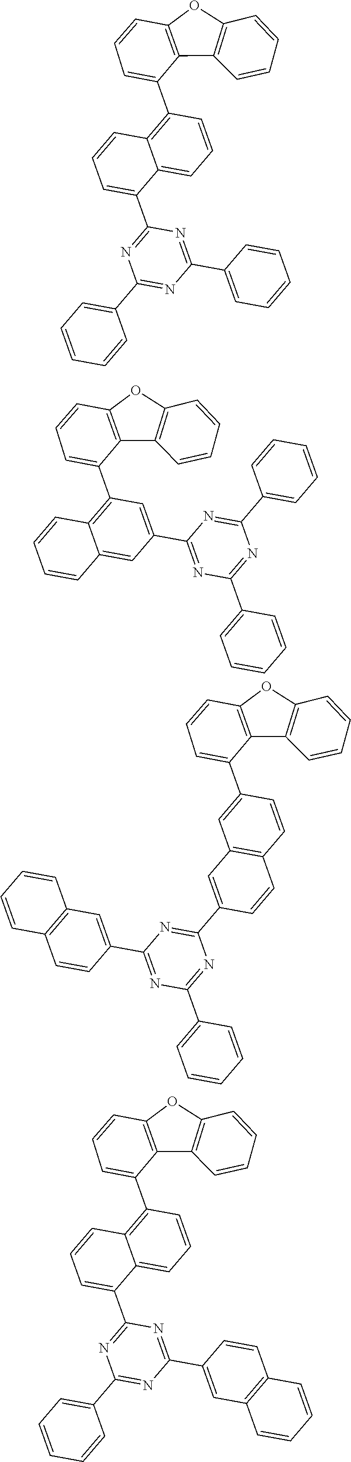

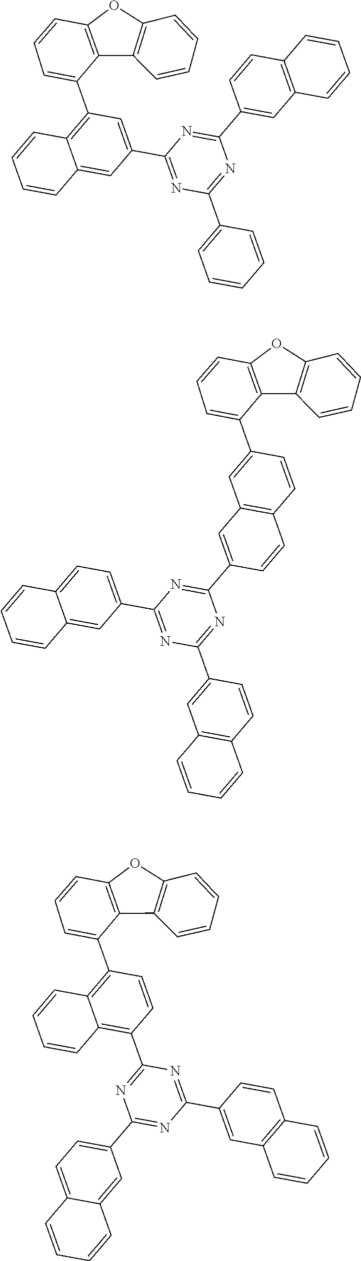 [SMILES: c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4ccc5ccc(-c6cccc7oc8ccccc8c67)cc5c4)n3)ccc2c1 ; c1ccc2cc(-c3nc(-c4ccc5ccccc5c4)nc(-c4ccc(-c5cccc6oc7ccccc7c56)c5ccccc45)n3)ccc2c1]

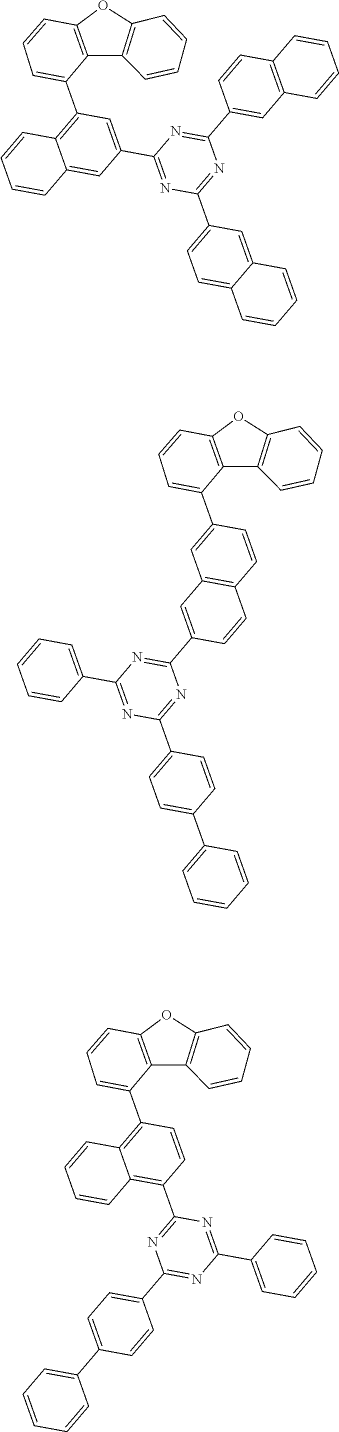 [SMILES: c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4ccc5ccc(-c6cccc7oc8ccccc8c67)cc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1]

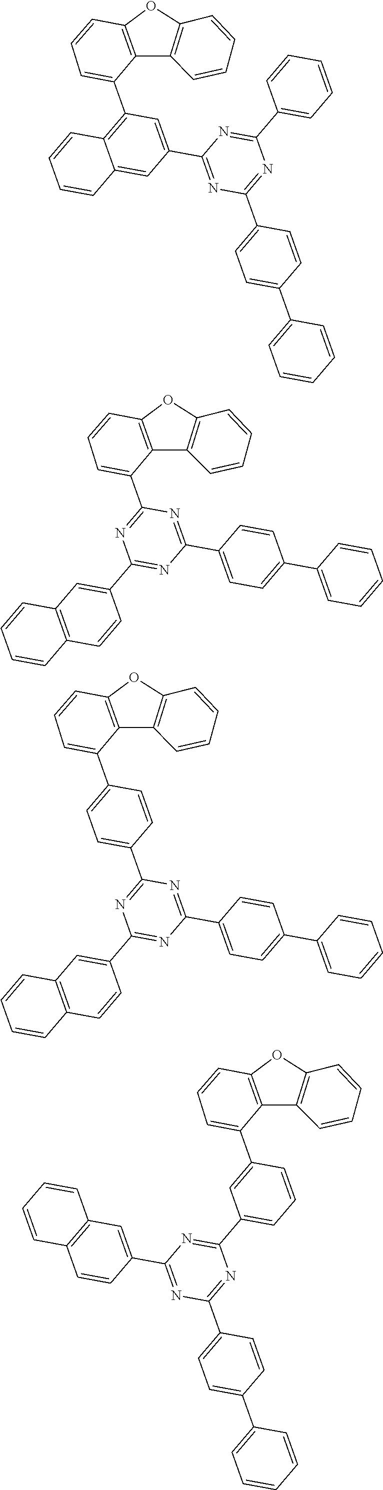

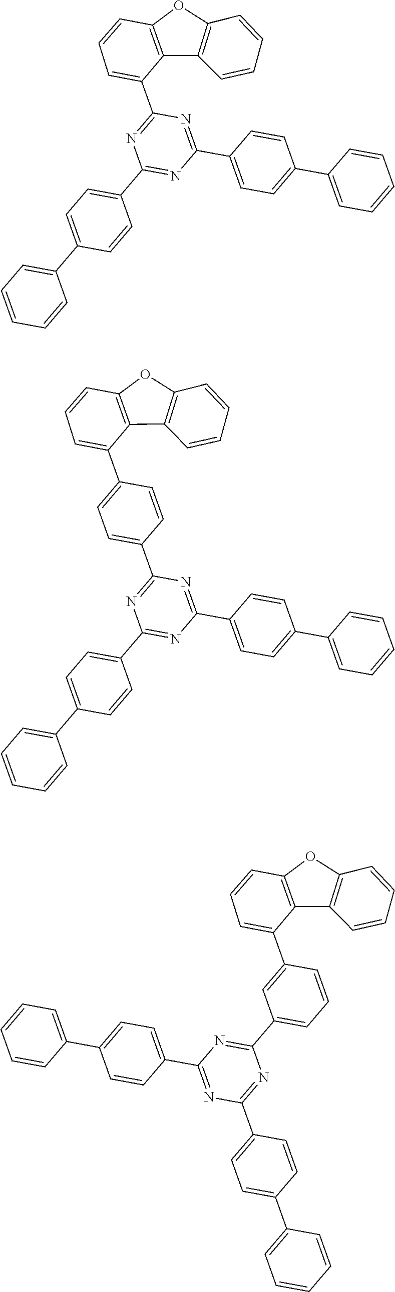 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1]

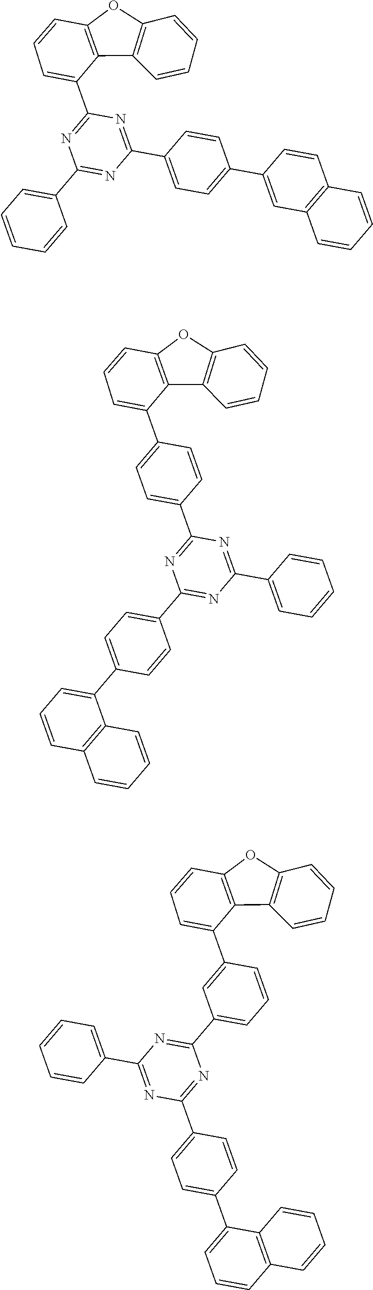 [SMILES: c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1]

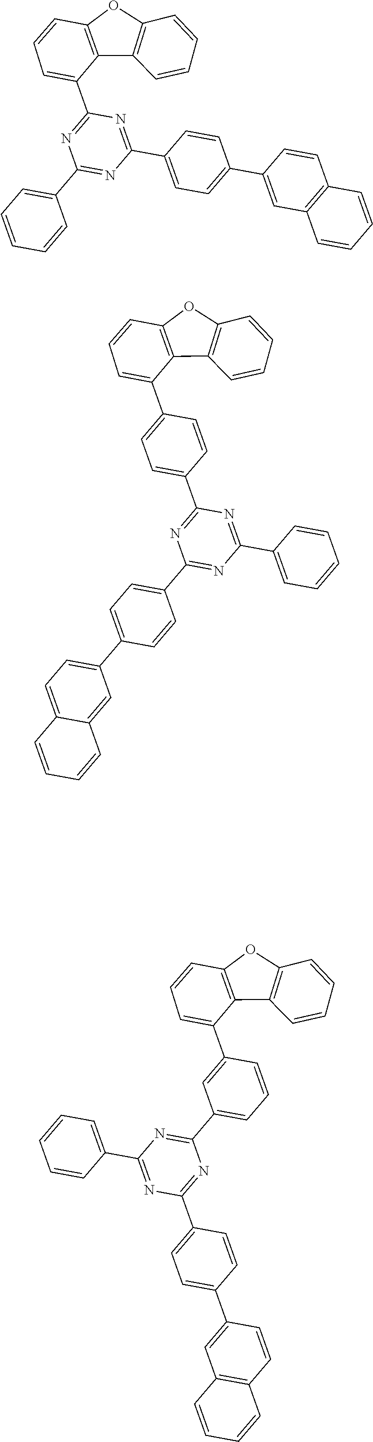 [SMILES: c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3cccc(-c4cccc5oc6ccccc6c45)c3)n2)cc1]

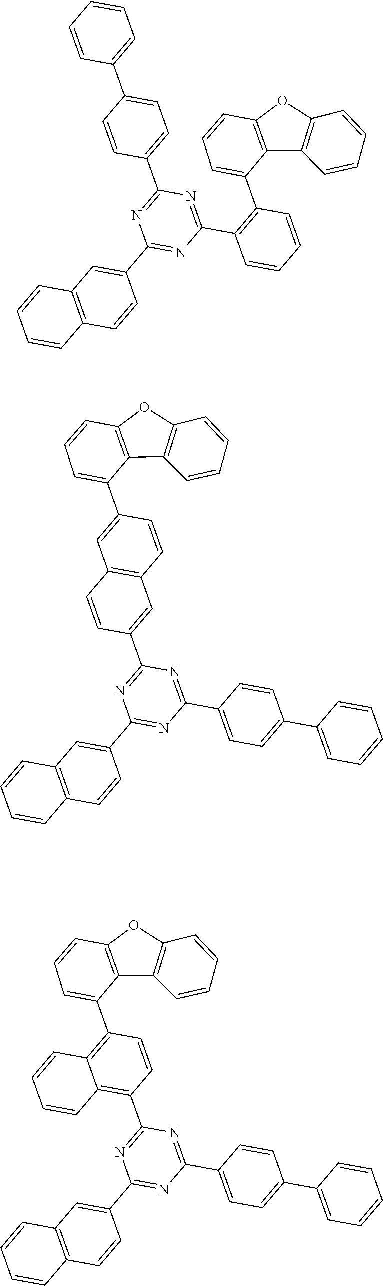 [SMILES: c1ccc(-c2ccc(-c3nc(-c4ccc5ccccc5c4)nc(-c4ccccc4-c4cccc5oc6ccccc6c45)n3)cc2)cc1 ; c1ccc(-c2ccc(-c3nc(-c4ccc5ccccc5c4)nc(-c4ccc5cc(-c6cccc7oc8ccccc8c67)ccc5c4)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1]

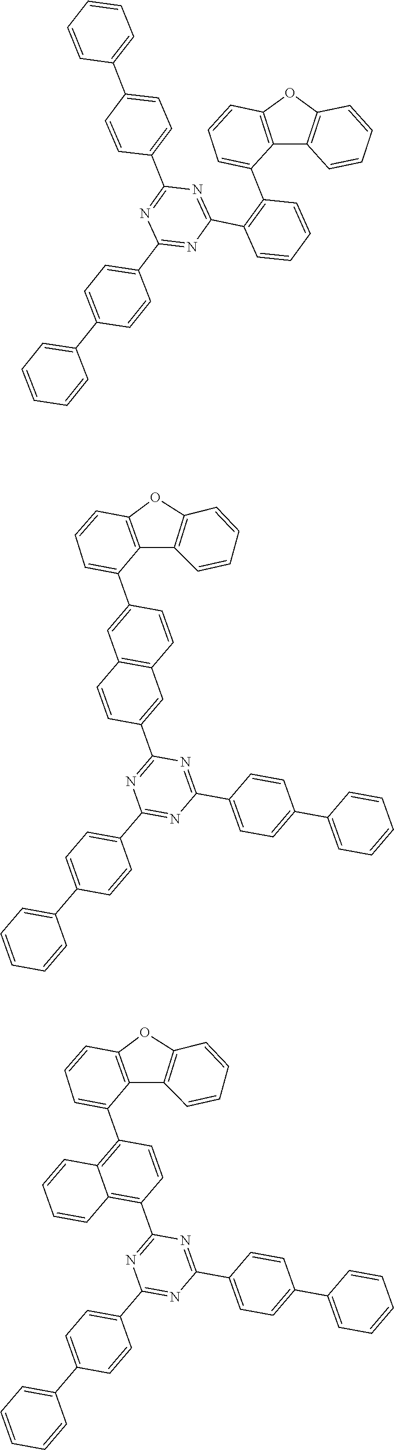 [SMILES: c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1]

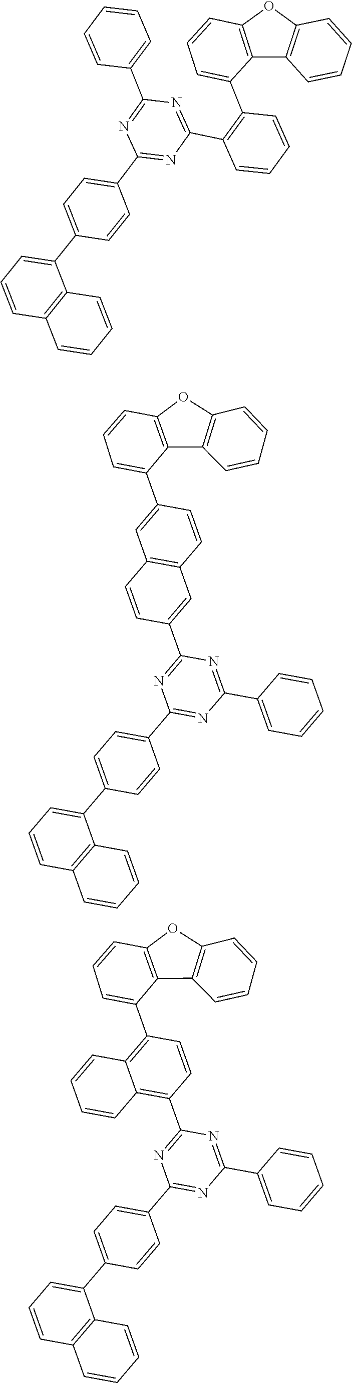

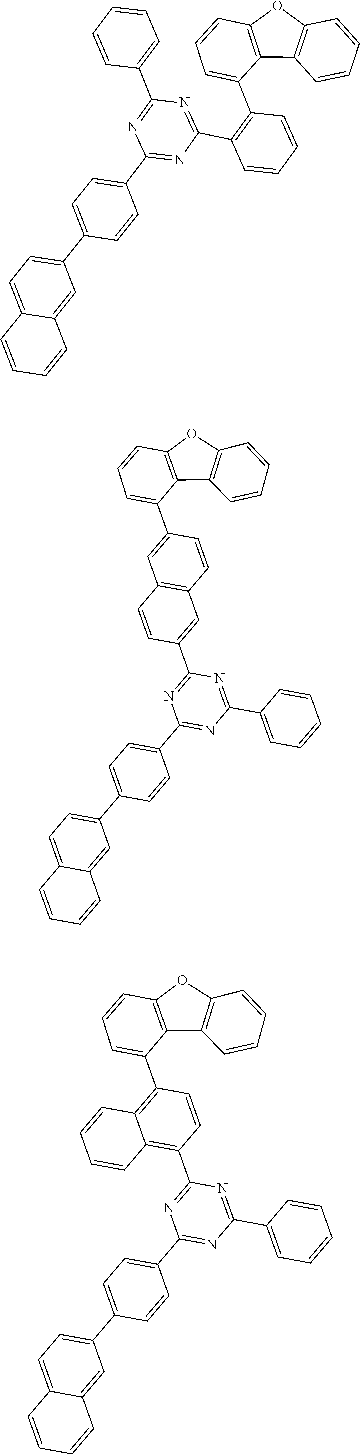 [SMILES: c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

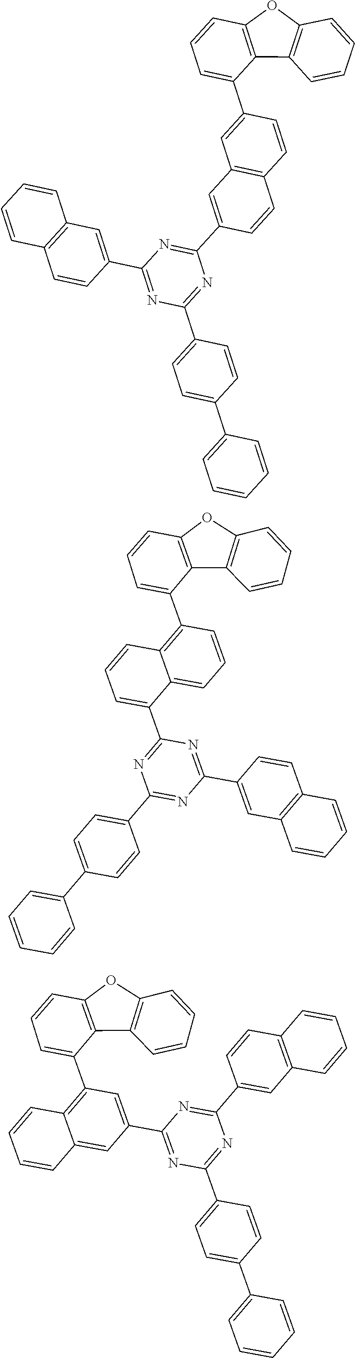

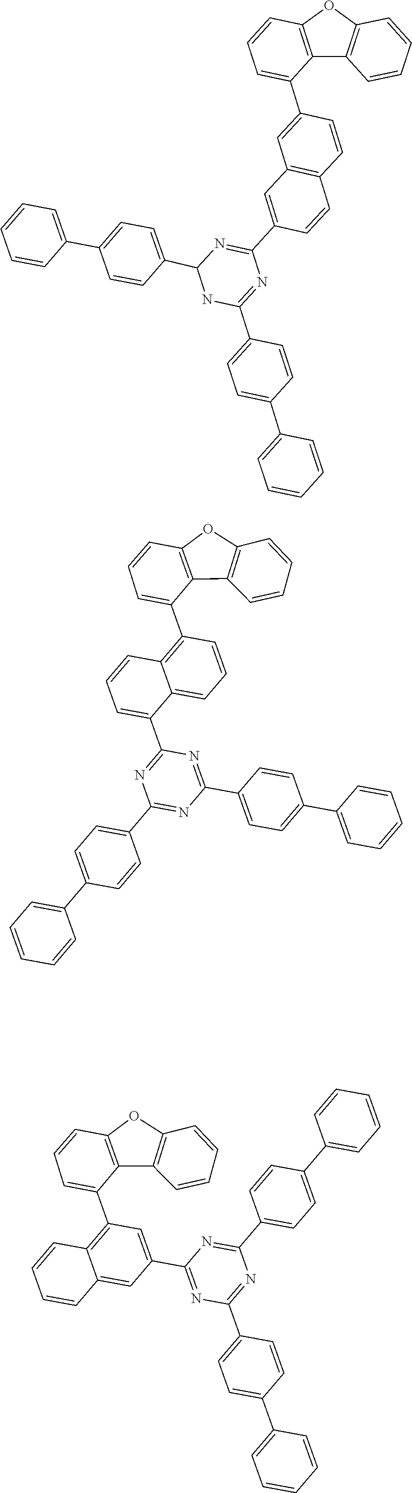

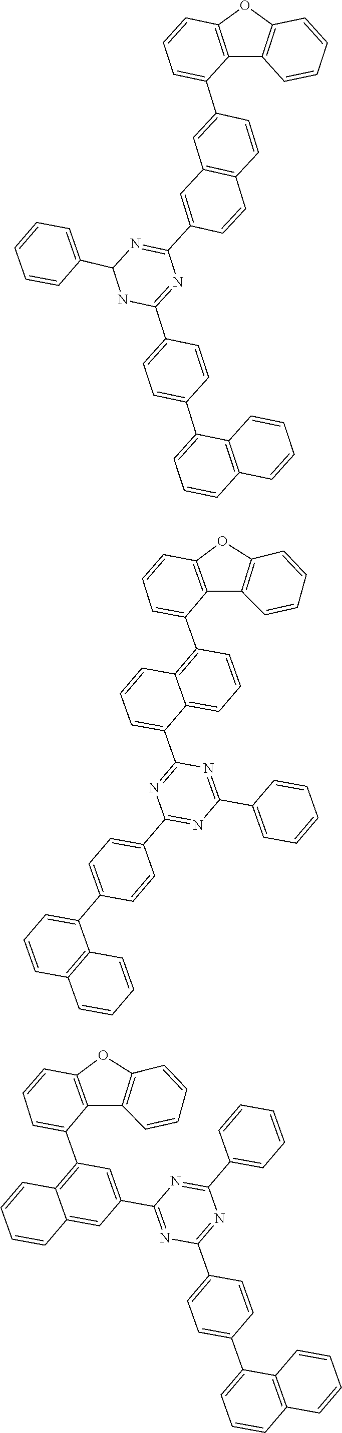

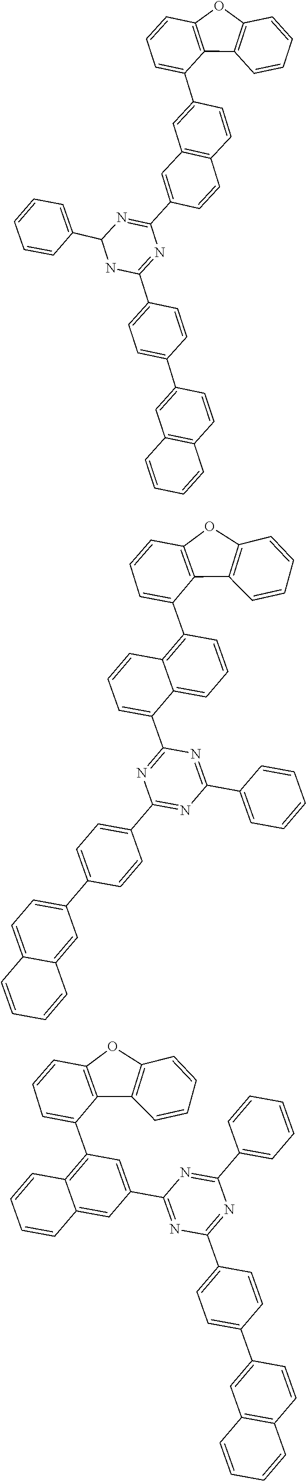

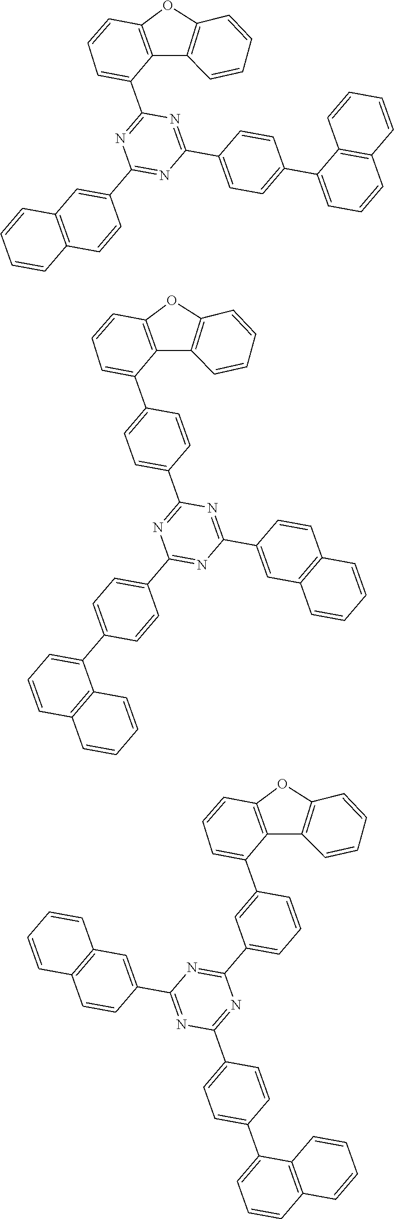 [SMILES: c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1 ; c1cc(-c2nc(-c3ccc(-c4cccc5ccccc45)cc3)nc(-c3ccc4ccccc4c3)n2)cc(-c2cccc3oc4ccccc4c23)c1]

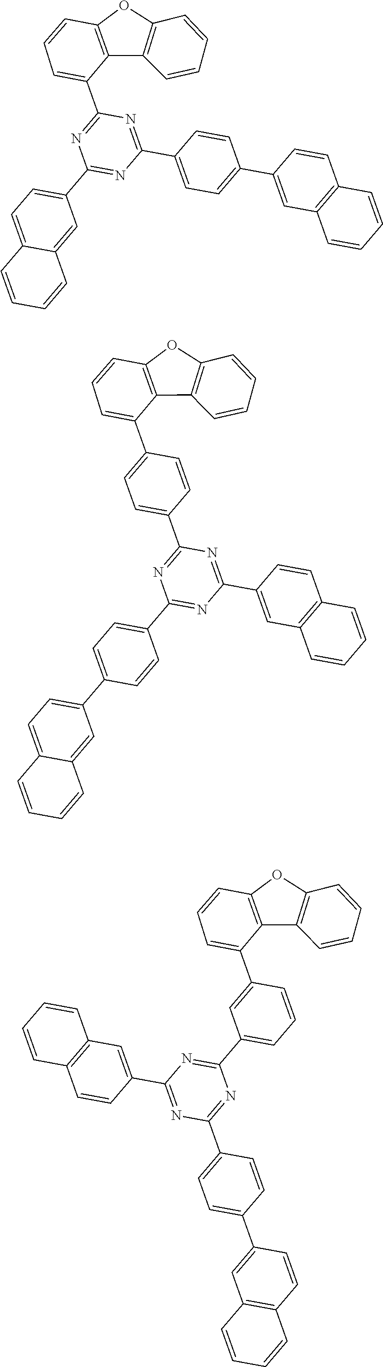 [SMILES: c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1]

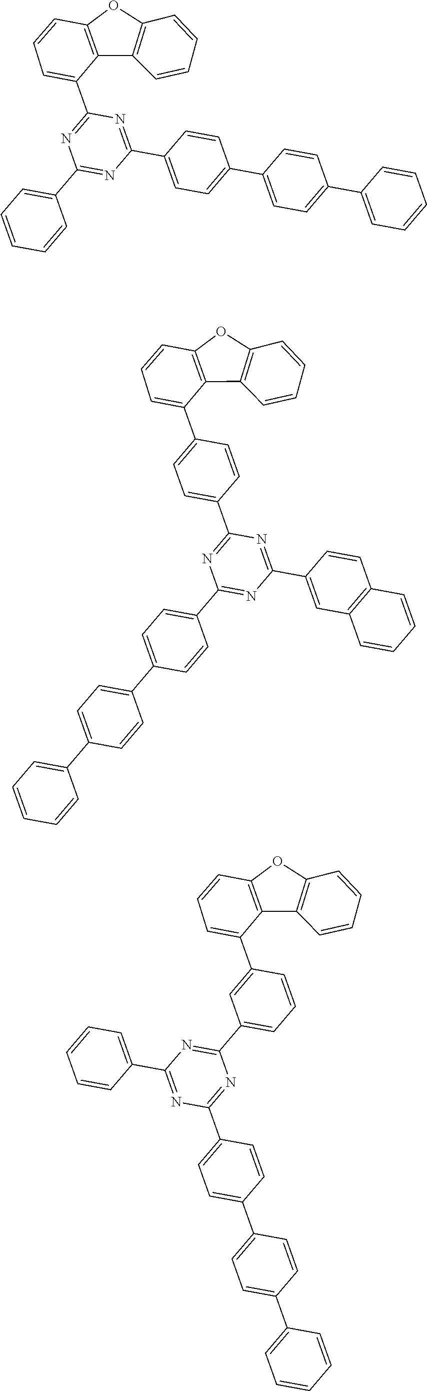 [SMILES: c1ccc(-c2ccc(-c3ccc(-c4nc(-c5ccccc5)nc(-c5cccc6oc7ccccc7c56)n4)cc3)cc2)cc1 ; c1ccc(-c2ccc(-c3ccc(-c4nc(-c5ccc(-c6cccc7oc8ccccc8c67)cc5)nc(-c5ccc6ccccc6c5)n4)cc3)cc2)cc1 ; c1ccc(-c2ccc(-c3ccc(-c4nc(-c5ccccc5)nc(-c5cccc(-c6cccc7oc8ccccc8c67)c5)n4)cc3)cc2)cc1]

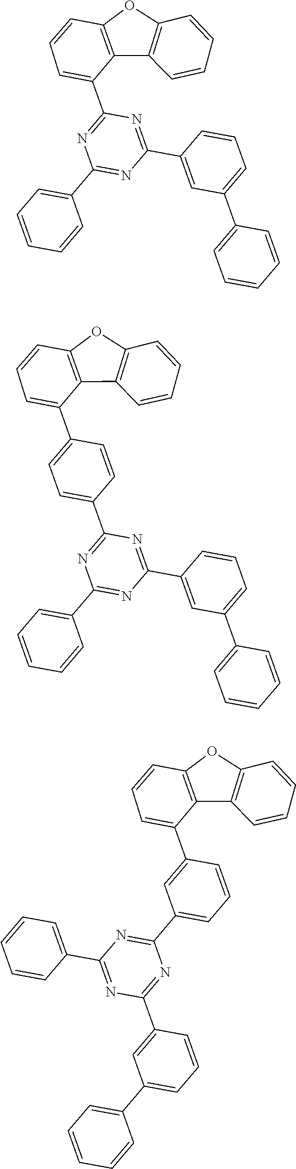 [SMILES: c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1 ; c1ccc(-c2cccc(-c3nc(-c4ccccc4)nc(-c4cccc(-c5cccc6oc7ccccc7c56)c4)n3)c2)cc1]

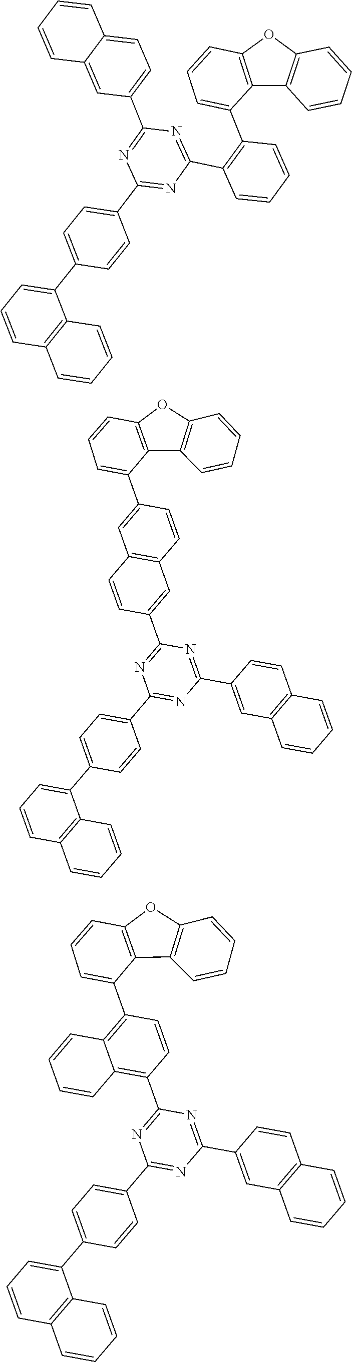

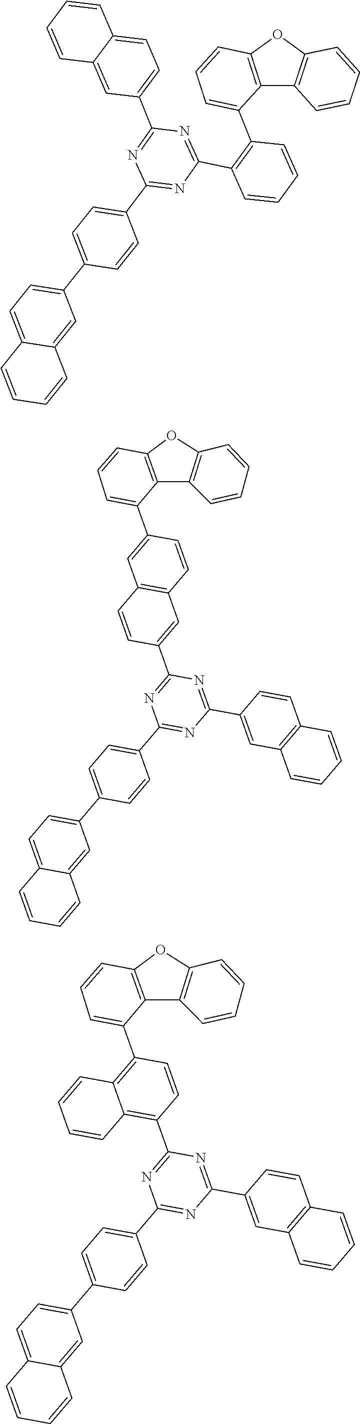 [SMILES: c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1 ; c1ccc2cc(-c3ccc(-c4nc(-c5ccc6ccccc6c5)nc(-c5ccc(-c6cccc7oc8ccccc8c67)c6ccccc56)n4)cc3)ccc2c1]

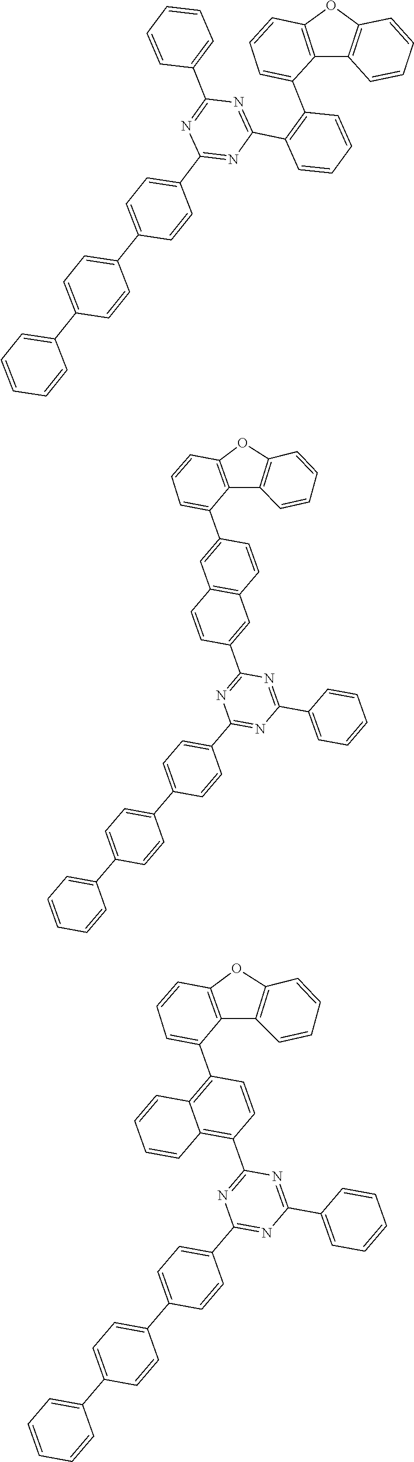 [SMILES: c1ccc(-c2ccc(-c3ccc(-c4nc(-c5ccccc5)nc(-c5ccccc5-c5cccc6oc7ccccc7c56)n4)cc3)cc2)cc1 ; c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

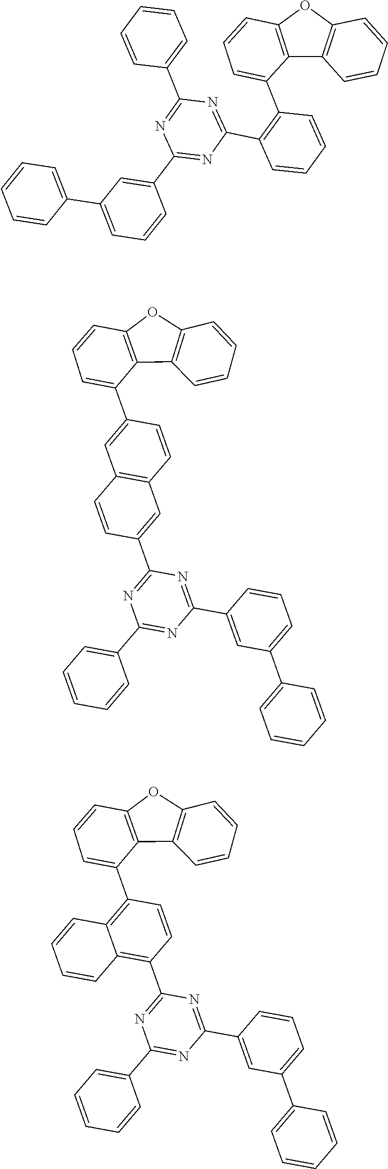 [SMILES: c1ccc(-c2cccc(-c3nc(-c4ccccc4)nc(-c4ccccc4-c4cccc5oc6ccccc6c45)n3)c2)cc1 ; c1ccc(-c2cccc(-c3nc(-c4ccccc4)nc(-c4ccc5cc(-c6cccc7oc8ccccc8c67)ccc5c4)n3)c2)cc1 ; c1ccc(-c2cccc(-c3nc(-c4ccccc4)nc(-c4ccc(-c5cccc6oc7ccccc7c56)c5ccccc45)n3)c2)cc1]

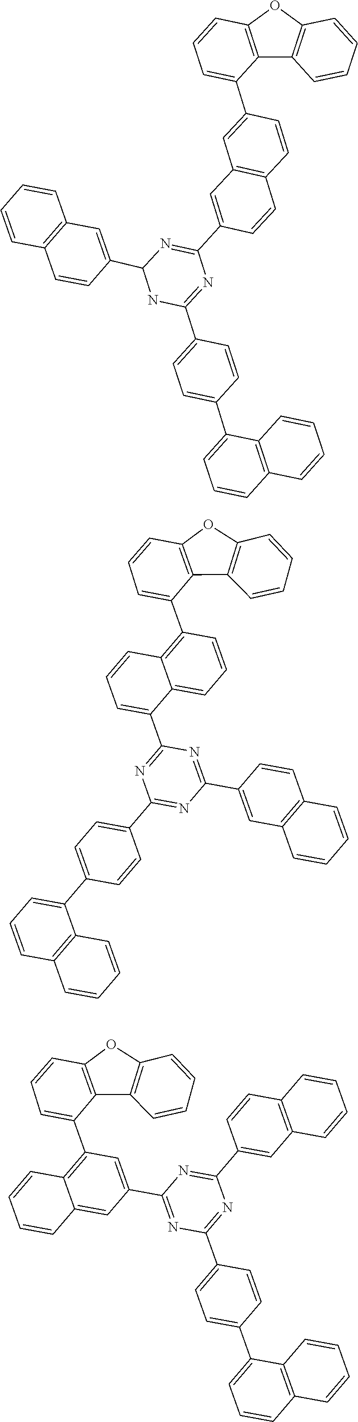

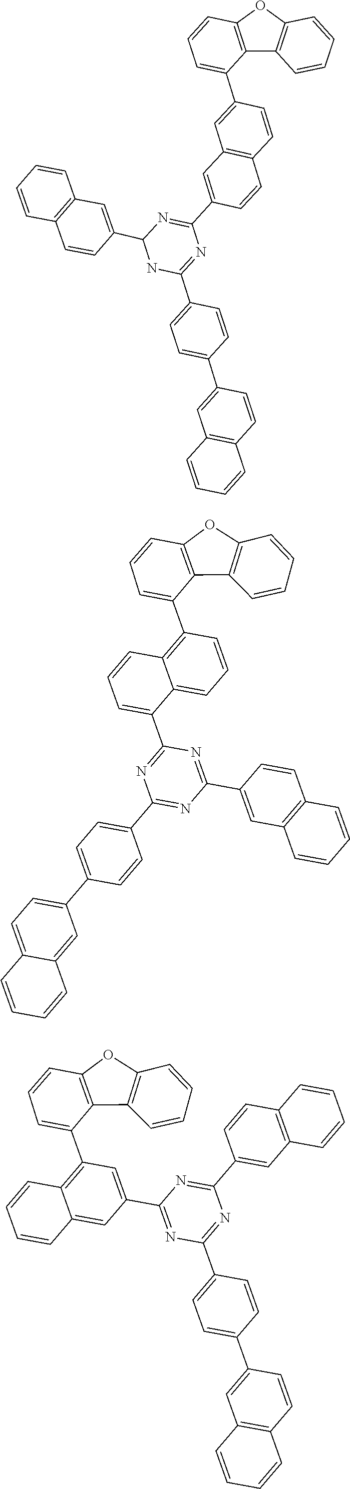

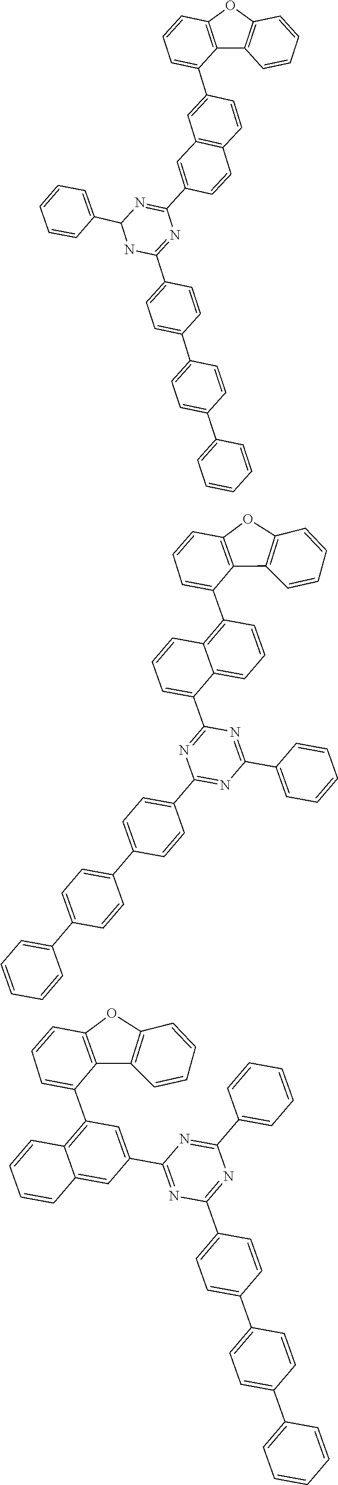

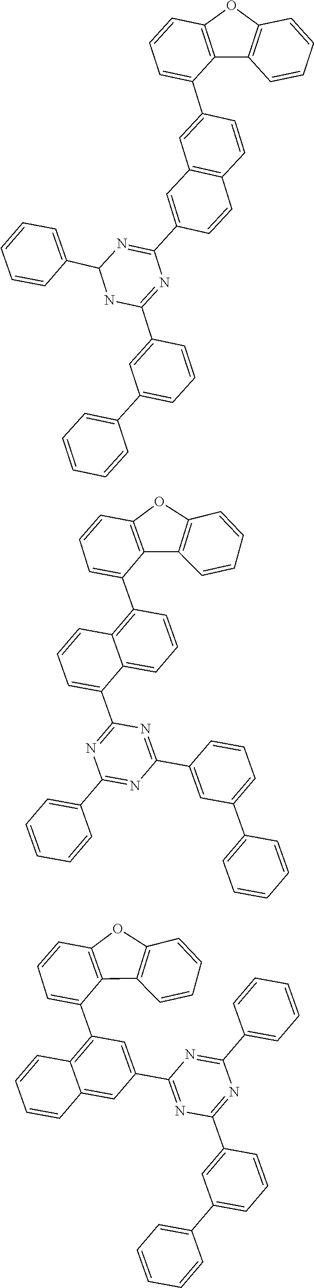

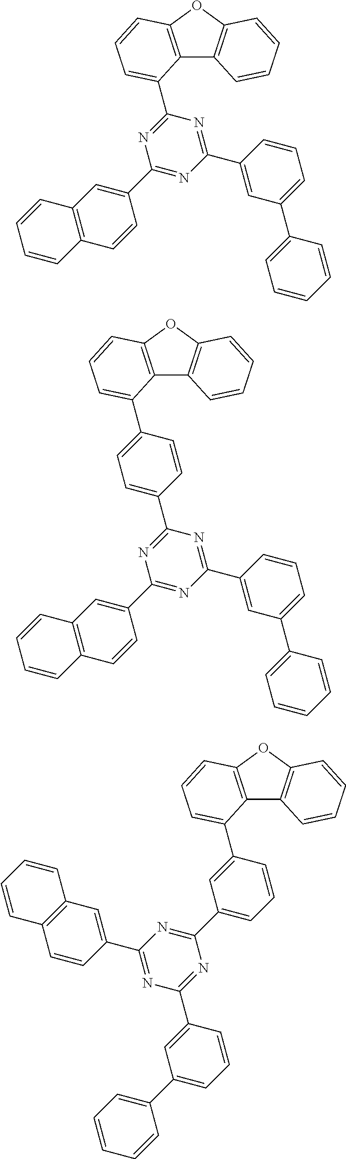

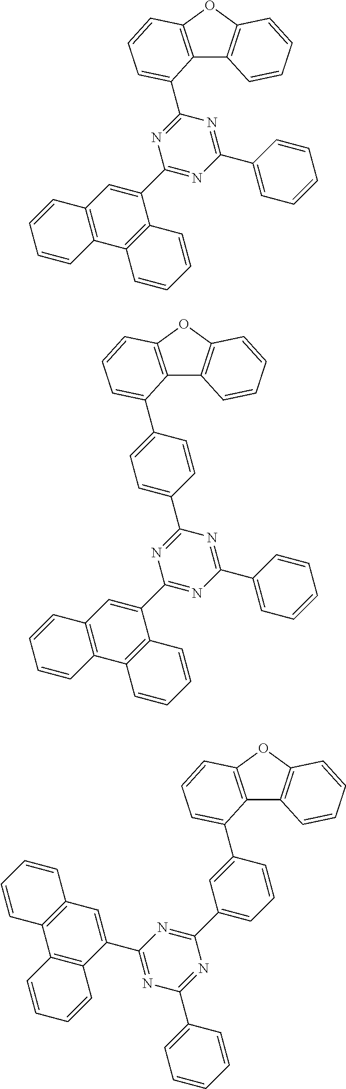

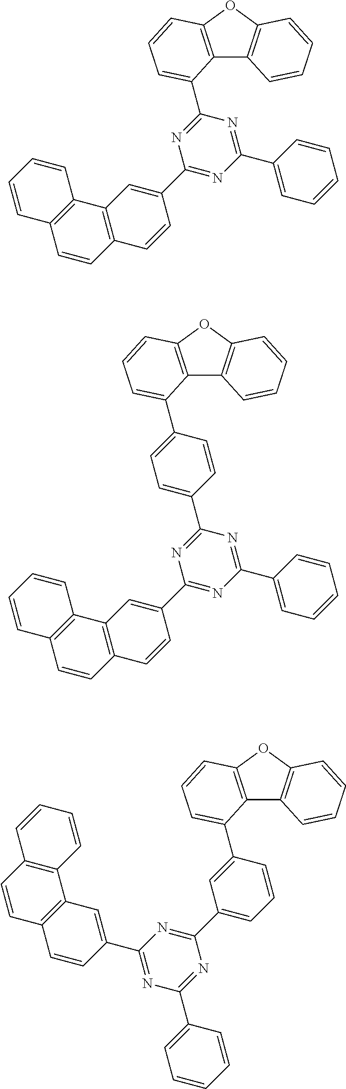 [SMILES: c1ccc(-c2nc(-c3ccc4ccc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1]

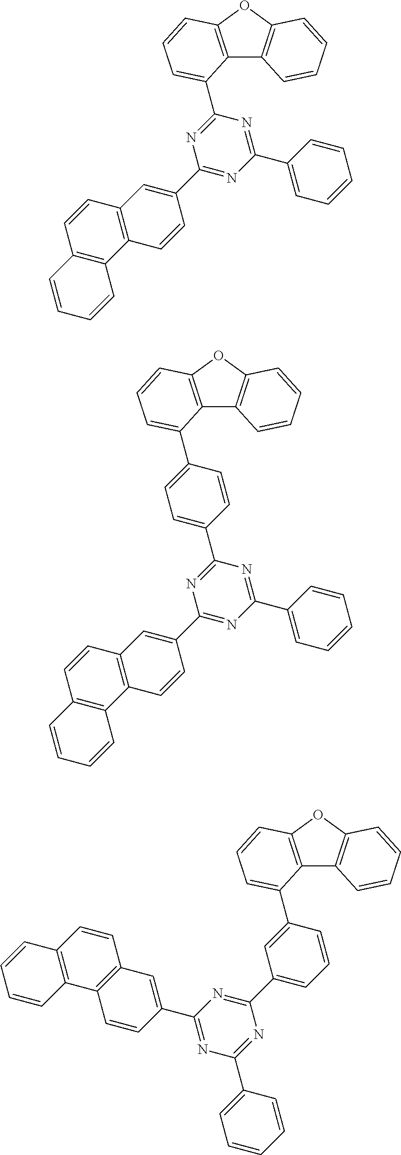 [SMILES: c1ccc(-c2nc(-c3ccc4c(ccc5ccccc54)c3)nc(-c3cccc4oc5ccccc5c34)n2)cc1 ; c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1]

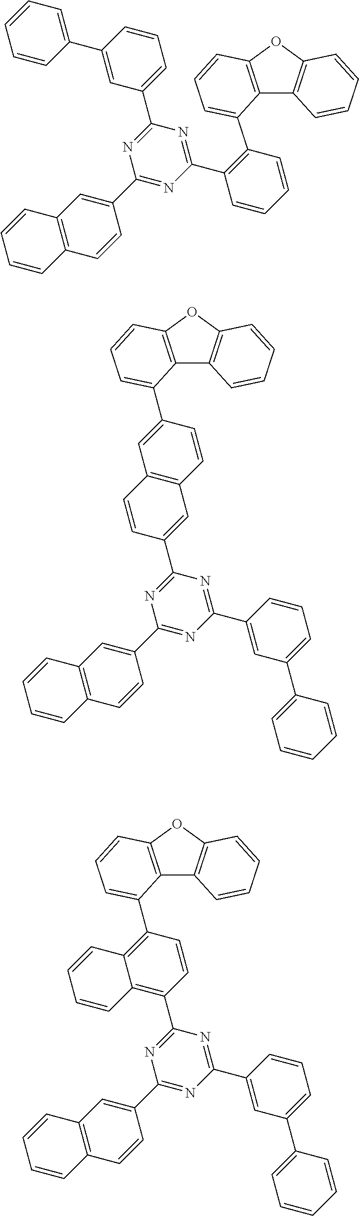 [SMILES: c1ccc(-c2cccc(-c3nc(-c4ccc5ccccc5c4)nc(-c4ccccc4-c4cccc5oc6ccccc6c45)n3)c2)cc1 ; c1ccc(-c2cccc(-c3nc(-c4ccc5ccccc5c4)nc(-c4ccc5cc(-c6cccc7oc8ccccc8c67)ccc5c4)n3)c2)cc1 ; c1ccc(-c2cccc(-c3nc(-c4ccc5ccccc5c4)nc(-c4ccc(-c5cccc6oc7ccccc7c56)c5ccccc45)n3)c2)cc1]

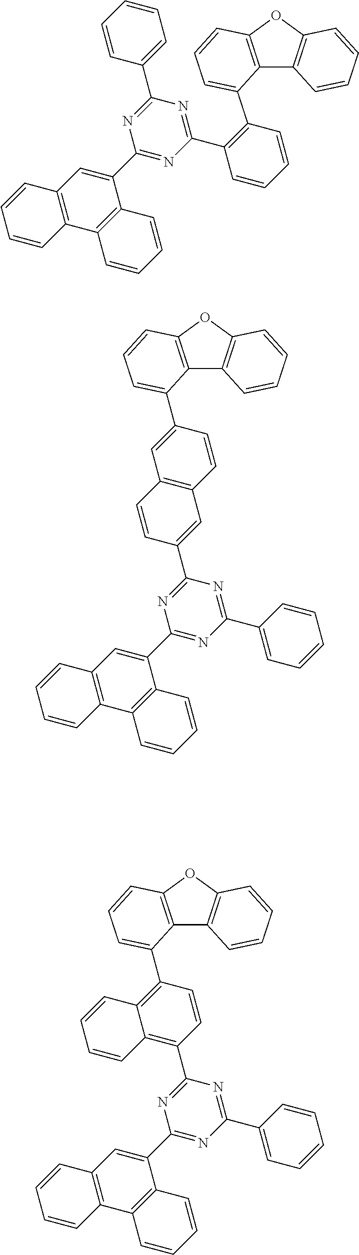 [SMILES: c1ccc(-c2nc(-c3ccccc3-c3cccc4oc5ccccc5c34)nc(-c3cc4ccccc4c4ccccc34)n2)cc1 ; c1ccc(-c2nc(-c3ccc4cc(-c5cccc6oc7ccccc7c56)ccc4c3)nc(-c3cc4ccccc4c4ccccc34)n2)cc1 ; c1ccc(-c2nc(-c3ccc(-c4cccc5oc6ccccc6c45)c4ccccc34)nc(-c3cc4ccccc4c4ccccc34)n2)cc1]

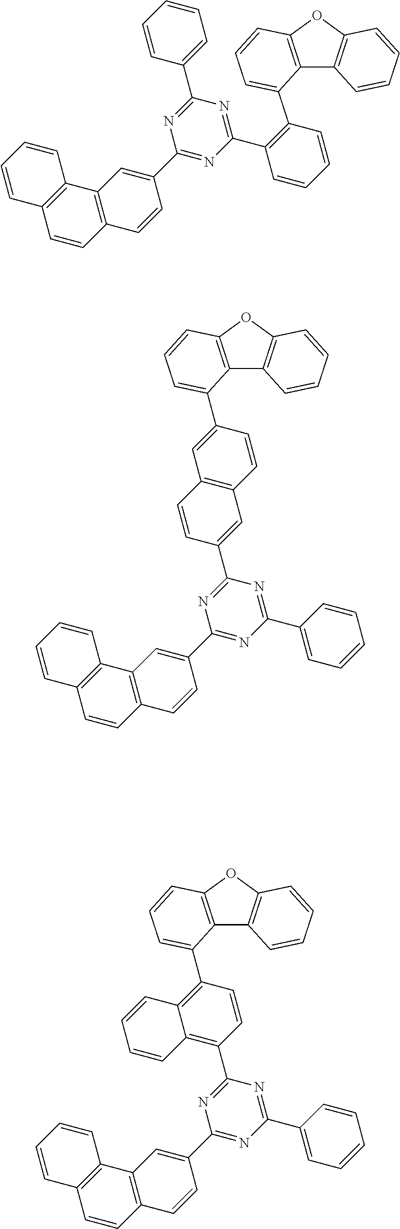 [SMILES: c1ccc(-c2nc(-c3ccc4ccc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1]

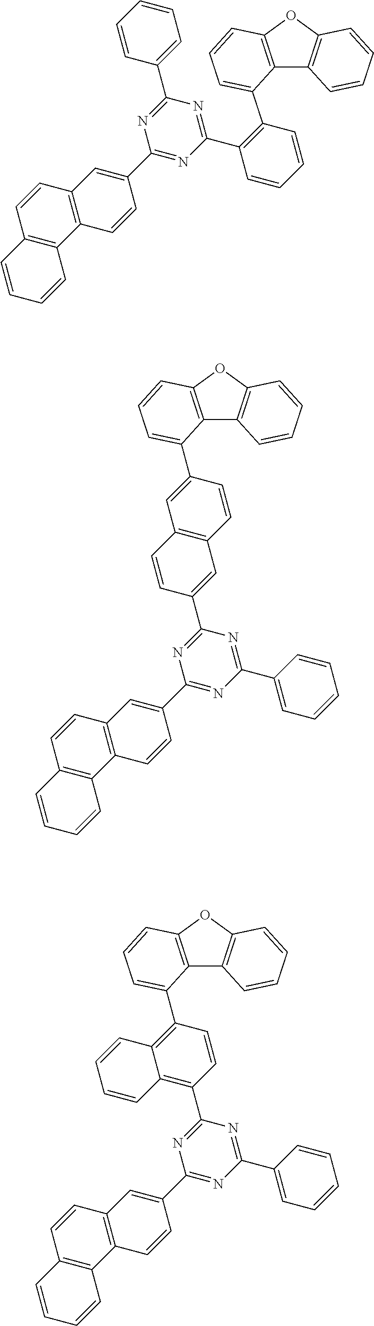 [SMILES: c1ccc(-c2nc(-c3ccc4c(ccc5ccccc54)c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1]

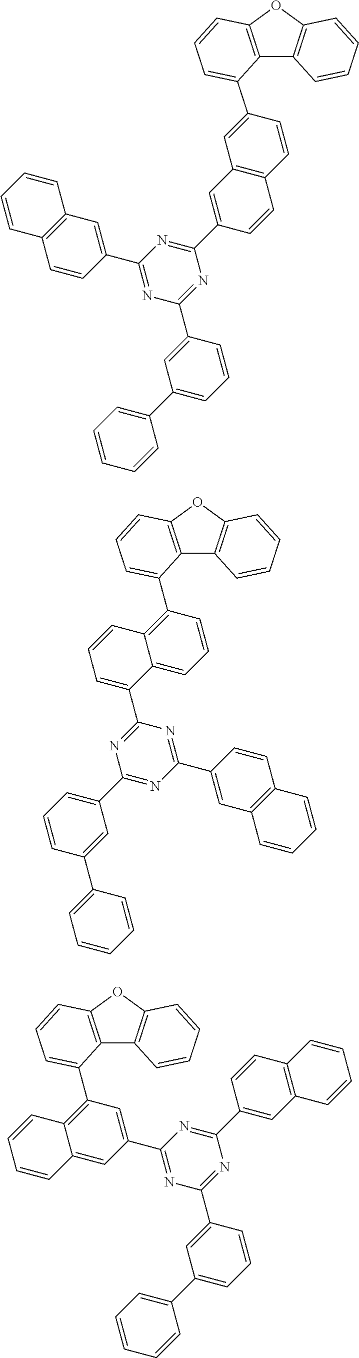

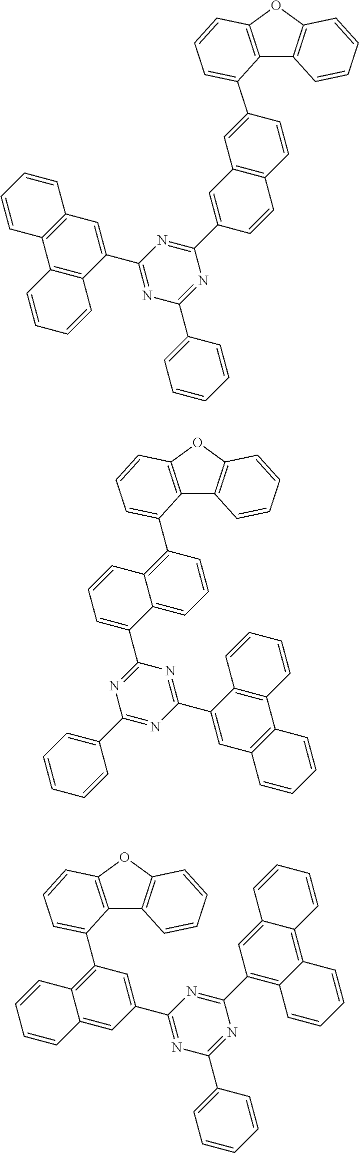

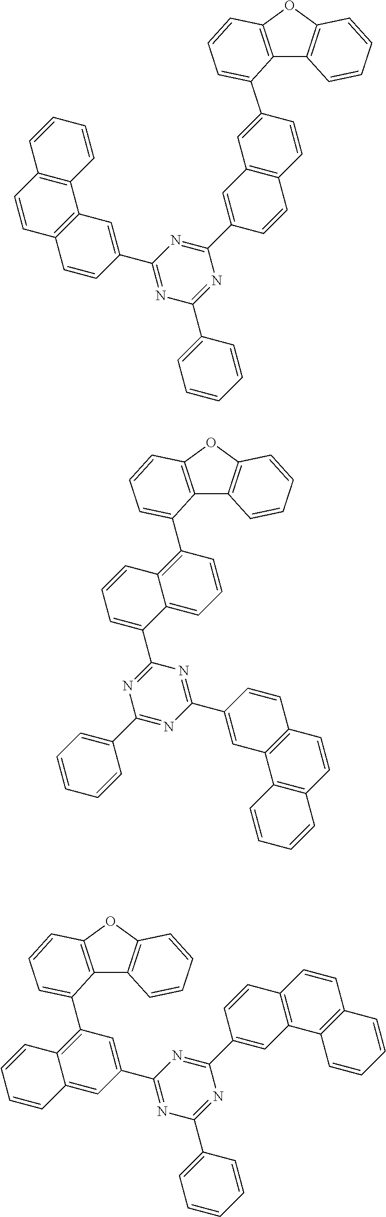

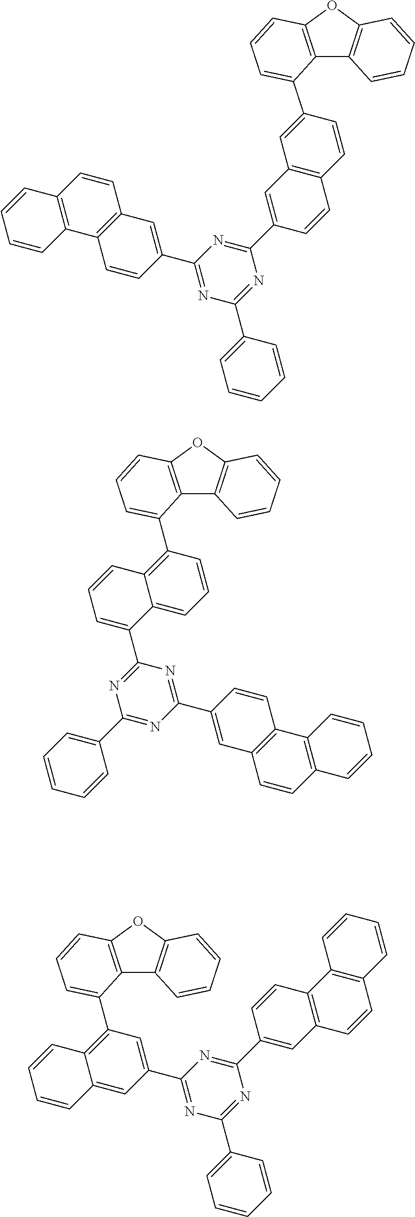 [SMILES: c1ccc(-c2nc(-c3ccc4ccc(-c5cccc6oc7ccccc7c56)cc4c3)nc(-c3ccc4c(ccc5ccccc54)c3)n2)cc1 ; c1ccc(-c2nc(-c3ccc4c(ccc5ccccc54)c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(ccc5ccccc54)c3)n2)cc1]

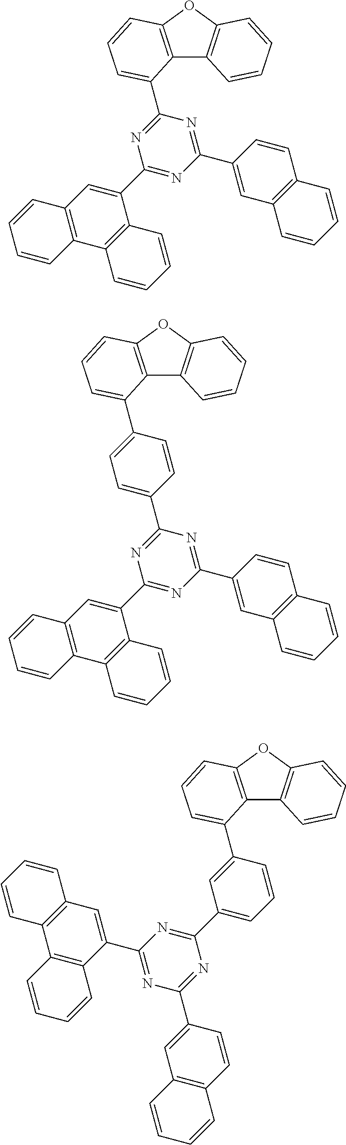

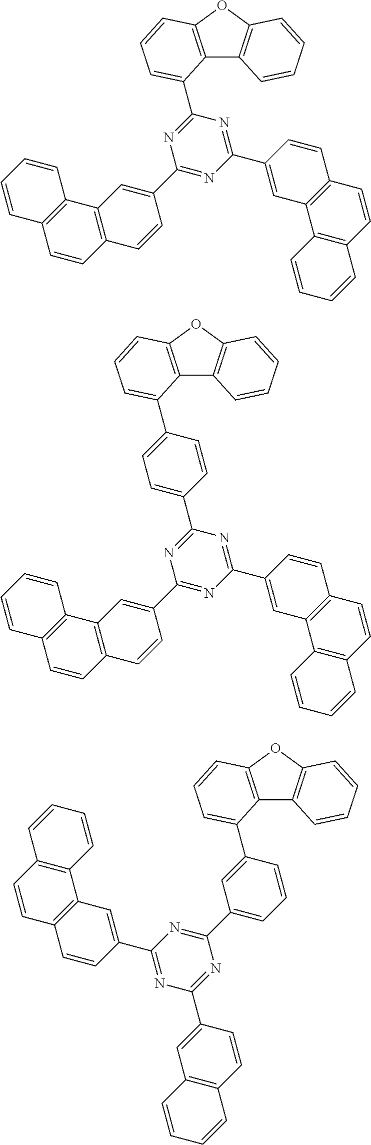

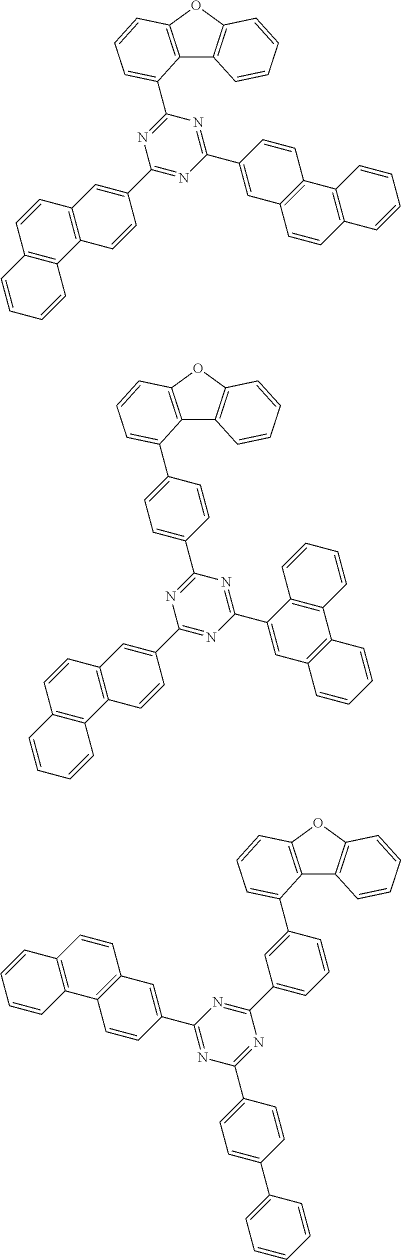 [SMILES: c1ccc2c(c1)ccc1cc(-c3nc(-c4ccc5c(ccc6ccccc65)c4)nc(-c4cccc5oc6ccccc6c45)n3)ccc12 ; c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1]

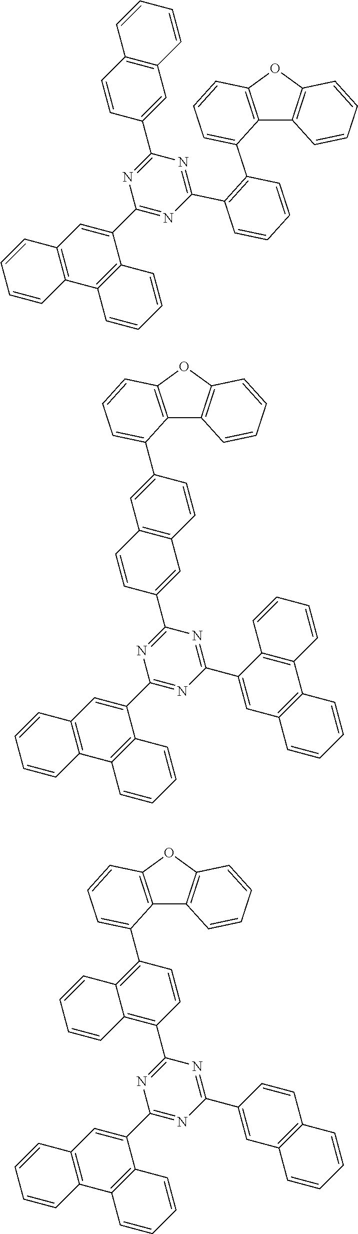 [SMILES: c1ccc(-c2cccc3oc4ccccc4c23)c(-c2nc(-c3ccc4ccccc4c3)nc(-c3cc4ccccc4c4ccccc34)n2)c1 ; c1ccc2c(c1)cc(-c1nc(-c3ccc4cc(-c5cccc6oc7ccccc7c56)ccc4c3)nc(-c3cc4ccccc4c4ccccc34)n1)c1ccccc12 ; c1ccc2cc(-c3nc(-c4ccc(-c5cccc6oc7ccccc7c56)c5ccccc45)nc(-c4cc5ccccc5c5ccccc45)n3)ccc2c1]

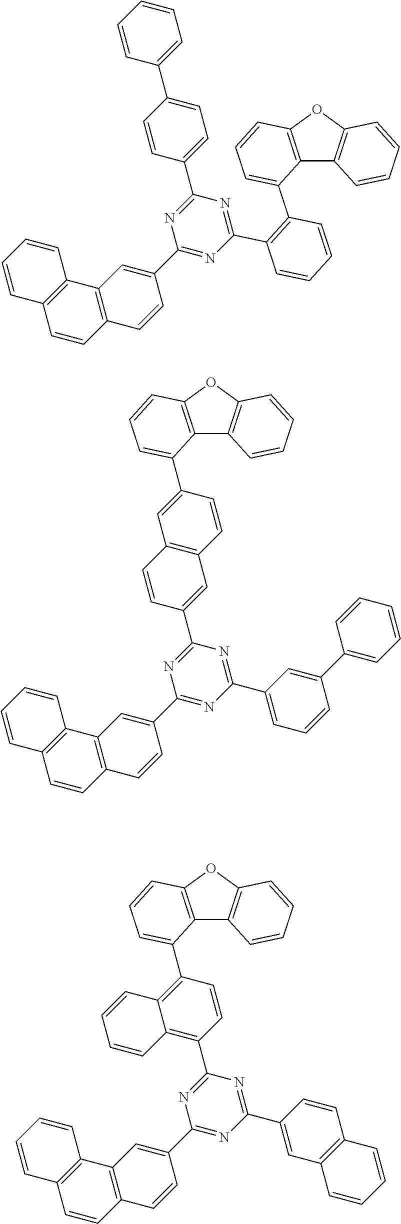 [SMILES: c1ccc(-c2ccc(-c3nc(-c4ccc5ccc6ccccc6c5c4)nc(-c4ccccc4-c4cccc5oc6ccccc6c45)n3)cc2)cc1 ; c1ccc(-c2cccc(-c3nc(-c4ccc5cc(-c6cccc7oc8ccccc8c67)ccc5c4)nc(-c4ccc5ccc6ccccc6c5c4)n3)c2)cc1 ; c1ccc2cc(-c3nc(-c4ccc5ccc6ccccc6c5c4)nc(-c4ccc(-c5cccc6oc7ccccc7c56)c5ccccc45)n3)ccc2c1]

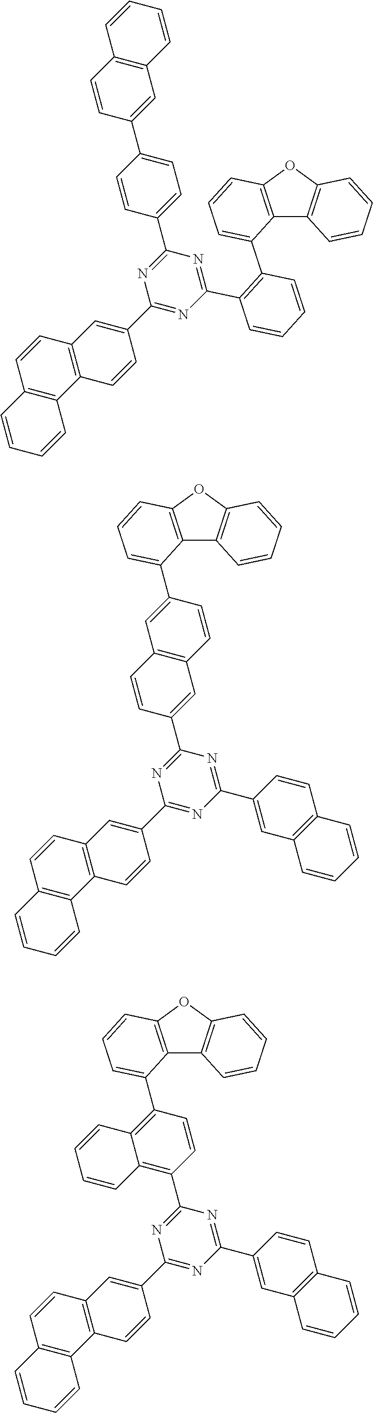 [SMILES: c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1]

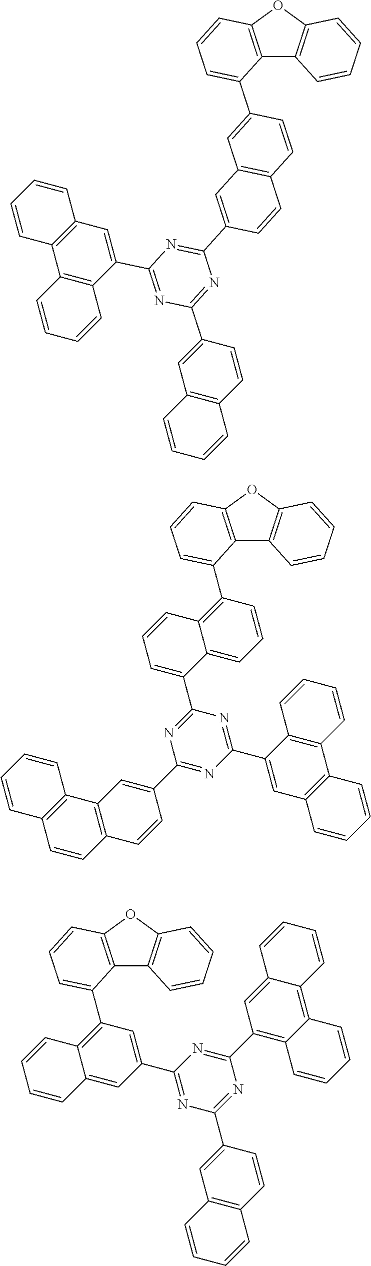

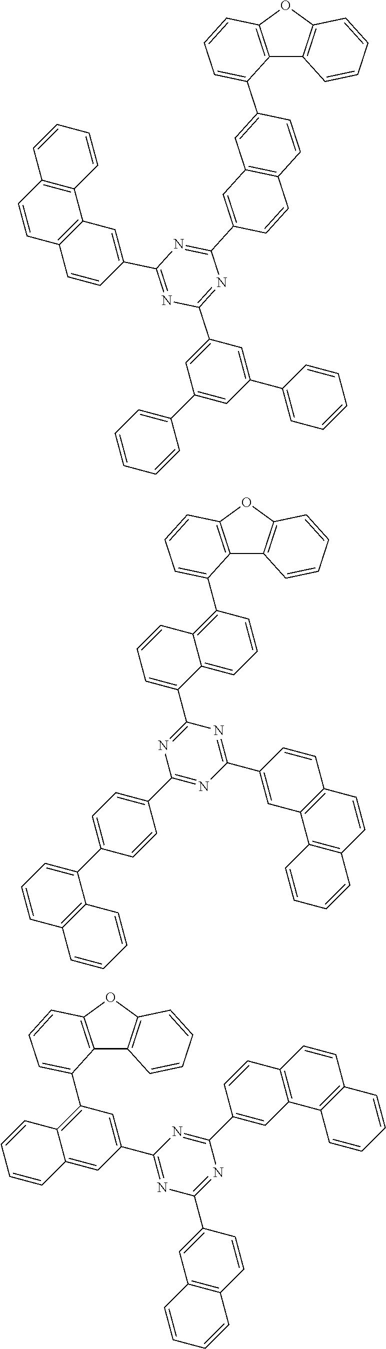

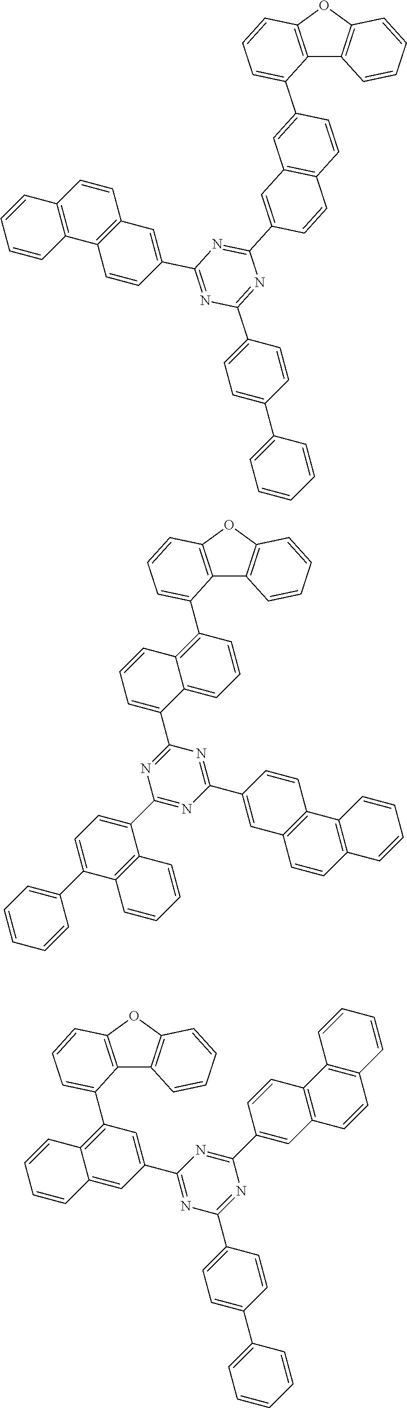

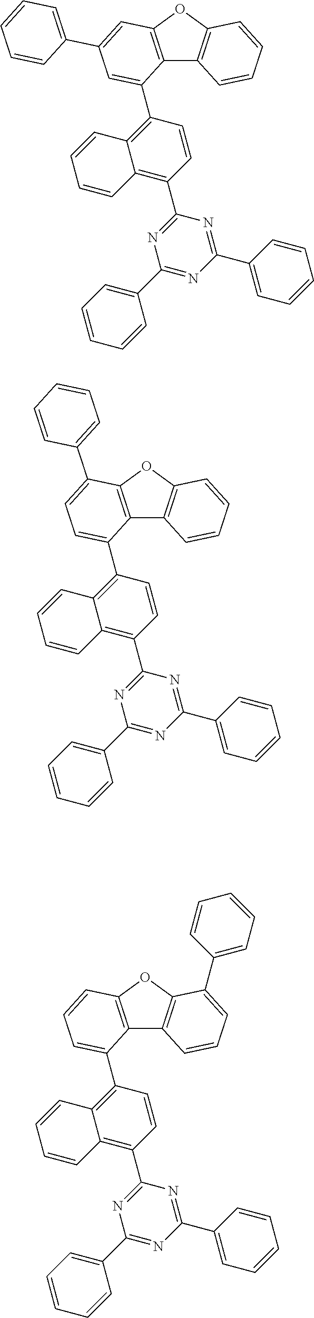 [SMILES: c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4ccc(-c5ccccc5)c5oc6ccccc6c45)c4ccccc34)n2)cc1 ; c1ccc(-c2nc(-c3ccccc3)nc(-c3ccc(-c4cccc5oc6c(-c7ccccc7)cccc6c45)c4ccccc34)n2)cc1]

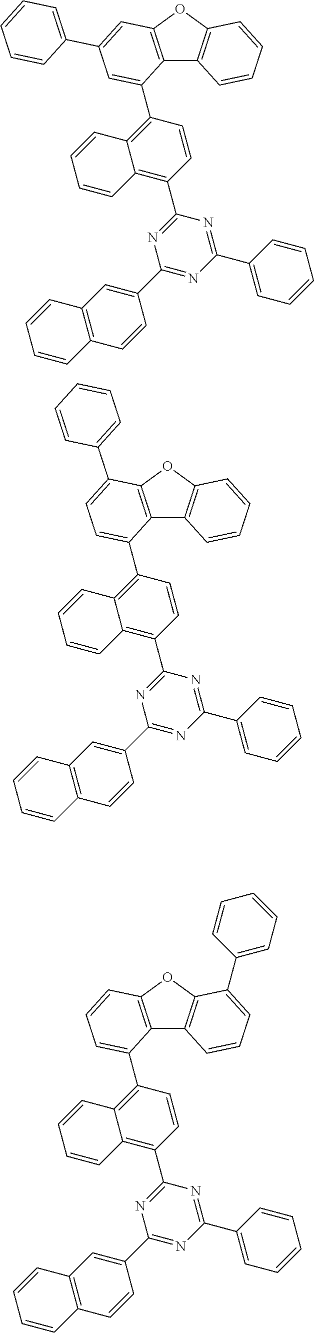

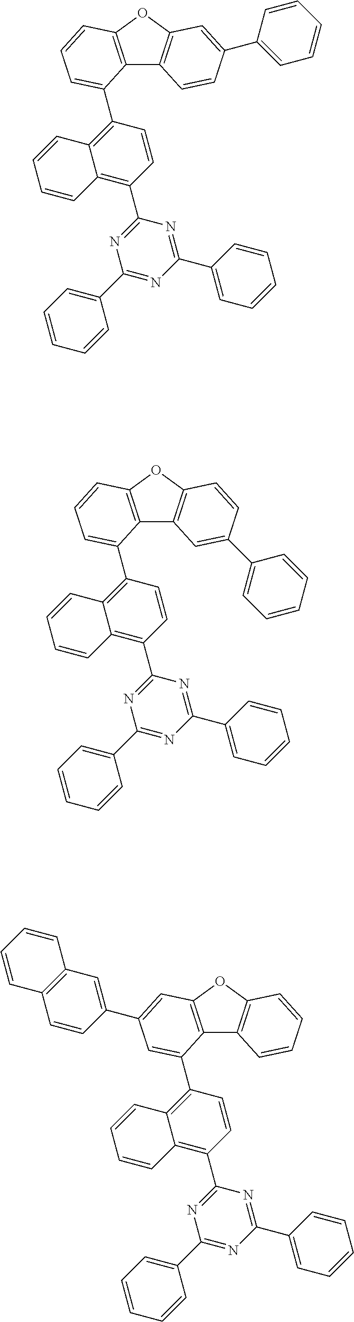 [SMILES: c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3oc4cccc(-c5ccc(-c6nc(-c7ccccc7)nc(-c7ccccc7)n6)c6ccccc56)c4c3c2)cc1 ; c1ccc(-c2nc(-c3ccccc3)nc(-c3ccc(-c4cc(-c5ccc6ccccc6c5)cc5oc6ccccc6c45)c4ccccc34)n2)cc1]

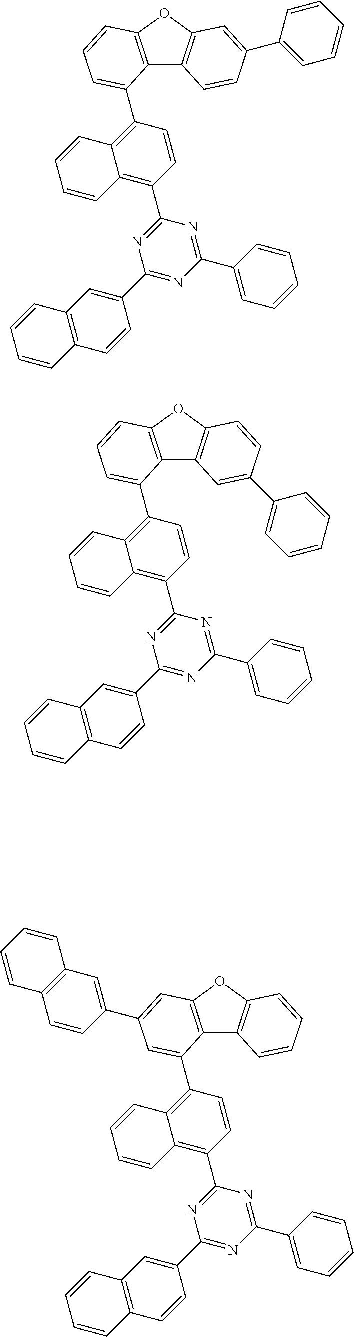 [SMILES: c1ccc(-c2ccc3c(c2)oc2cccc(-c4ccc(-c5nc(-c6ccccc6)nc(-c6ccc7ccccc7c6)n5)c5ccccc45)c23)cc1 ; c1ccc(-c2ccc3oc4cccc(-c5ccc(-c6nc(-c7ccccc7)nc(-c7ccc8ccccc8c7)n6)c6ccccc56)c4c3c2)cc1 ; c1ccc(-c2nc(-c3ccc4ccccc4c3)nc(-c3ccc(-c4cc(-c5ccc6ccccc6c5)cc5oc6ccccc6c45)c4ccccc34)n2)cc1]

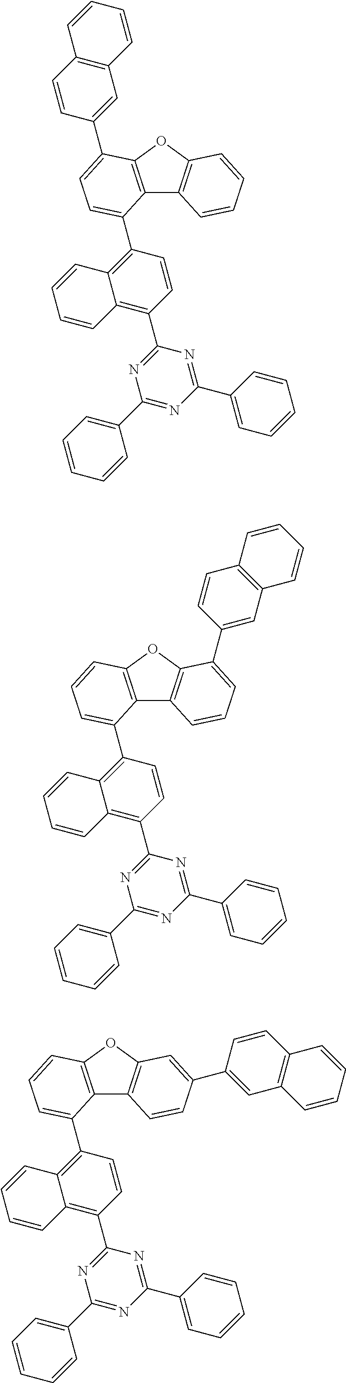 [SMILES: c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1 ; c1ccc(-c2nc(-c3ccccc3)nc(-c3ccc(-c4cccc5oc6cc(-c7ccc8ccccc8c7)ccc6c45)c4ccccc34)n2)cc1]

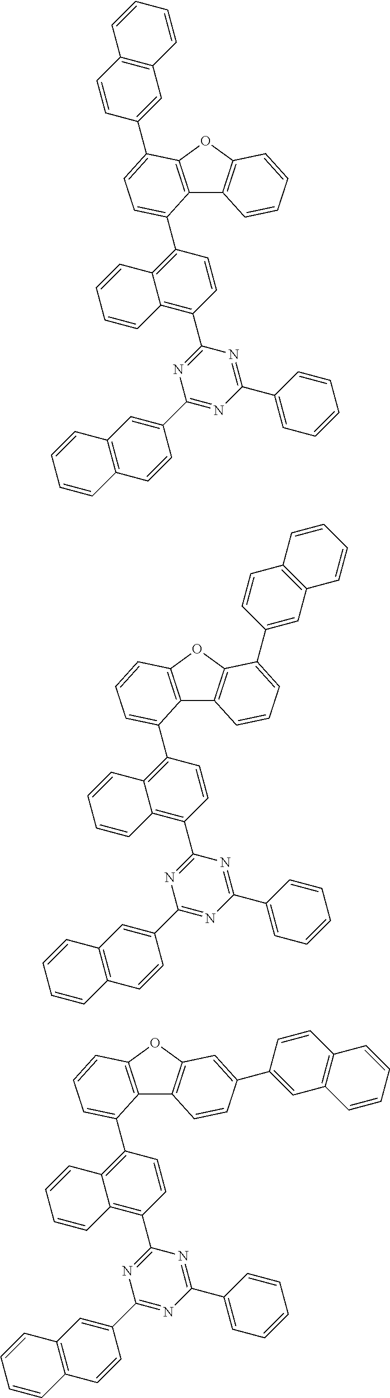 [SMILES: c1ccc(-c2nc(-c3ccc4ccccc4c3)nc(-c3ccc(-c4ccc(-c5ccc6ccccc6c5)c5oc6ccccc6c45)c4ccccc34)n2)cc1 ; c1ccc(-c2nc(-c3ccc4ccccc4c3)nc(-c3ccc(-c4cccc5oc6c(-c7ccc8ccccc8c7)cccc6c45)c4ccccc34)n2)cc1 ; c1ccc(-c2nc(-c3ccc4ccccc4c3)nc(-c3ccc(-c4cccc5oc6cc(-c7ccc8ccccc8c7)ccc6c45)c4ccccc34)n2)cc1]

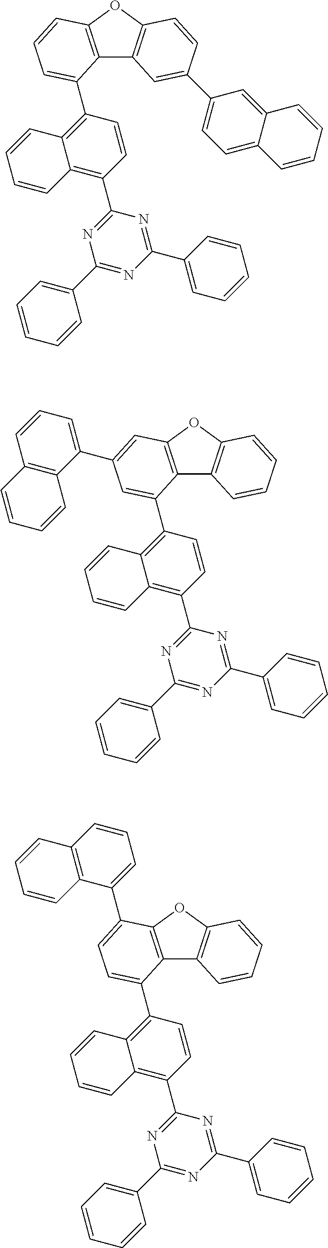 [SMILES: c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccccc3)nc(-c3ccc(-c4ccc(-c5cccc6ccccc56)c5oc6ccccc6c45)c4ccccc34)n2)cc1]

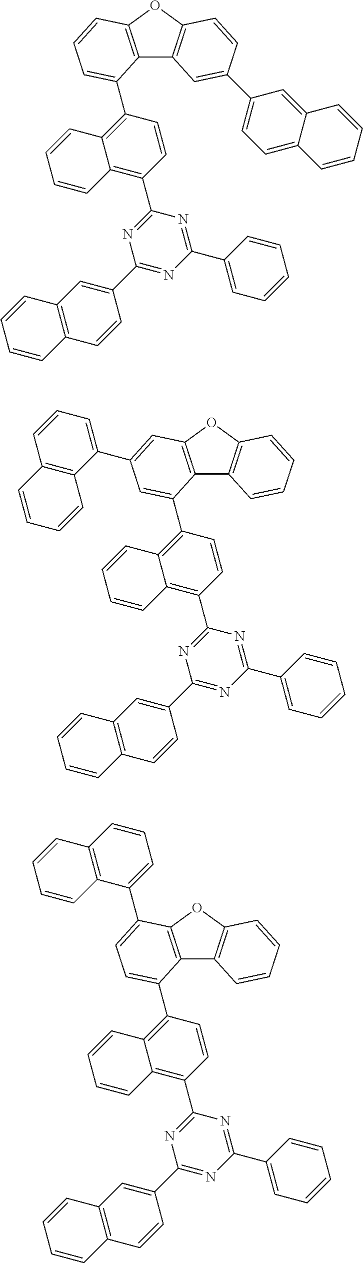 [SMILES: c1ccc(-c2nc(-c3ccc4ccccc4c3)nc(-c3ccc(-c4cccc5oc6ccc(-c7ccc8ccccc8c7)cc6c45)c4ccccc34)n2)cc1 ; c1ccc(-c2nc(-c3ccc4ccccc4c3)nc(-c3ccc(-c4cc(-c5cccc6ccccc56)cc5oc6ccccc6c45)c4ccccc34)n2)cc1 ; c1ccc(-c2nc(-c3ccc4ccccc4c3)nc(-c3ccc(-c4ccc(-c5cccc6ccccc56)c5oc6ccccc6c45)c4ccccc34)n2)cc1]

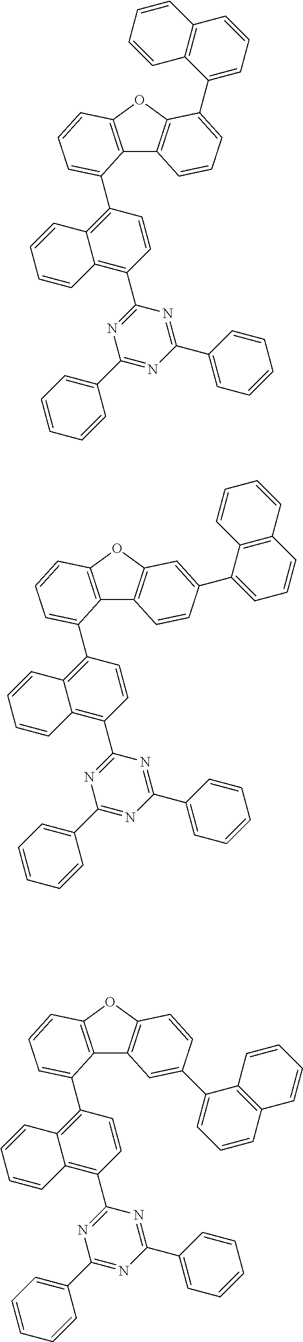 [SMILES: c1ccc(-c2nc(-c3ccccc3)nc(-c3ccc(-c4cccc5oc6c(-c7cccc8ccccc78)cccc6c45)c4ccccc34)n2)cc1 ; c1ccc(-c2nc(-c3ccccc3)nc(-c3ccc(-c4cccc5oc6cc(-c7cccc8ccccc78)ccc6c45)c4ccccc34)n2)cc1 ; c1ccc(-c2nc(-c3ccccc3)nc(-c3ccc(-c4cccc5oc6ccc(-c7cccc8ccccc78)cc6c45)c4ccccc34)n2)cc1]

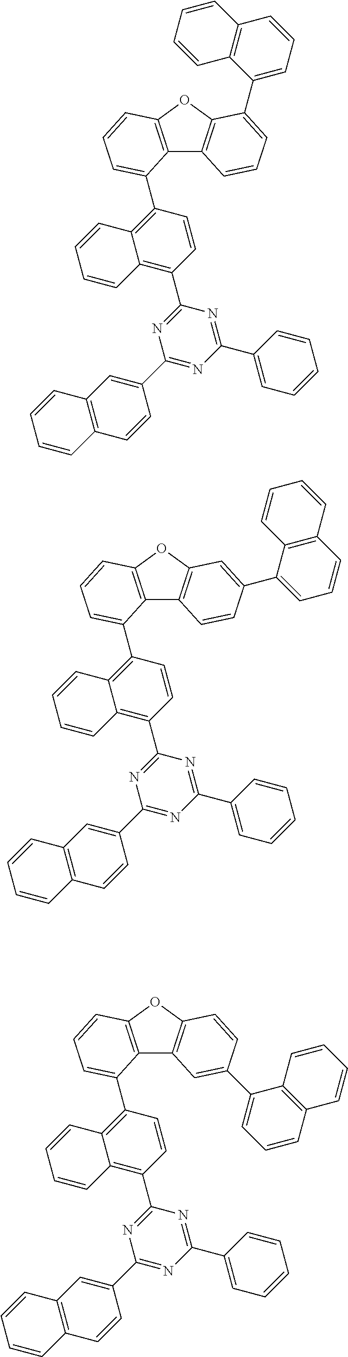 [SMILES: c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1 ; c1ccc(-c2nc(-c3ccc4ccccc4c3)nc(-c3ccc(-c4cccc5oc6ccc(-c7cccc8ccccc78)cc6c45)c4ccccc34)n2)cc1]

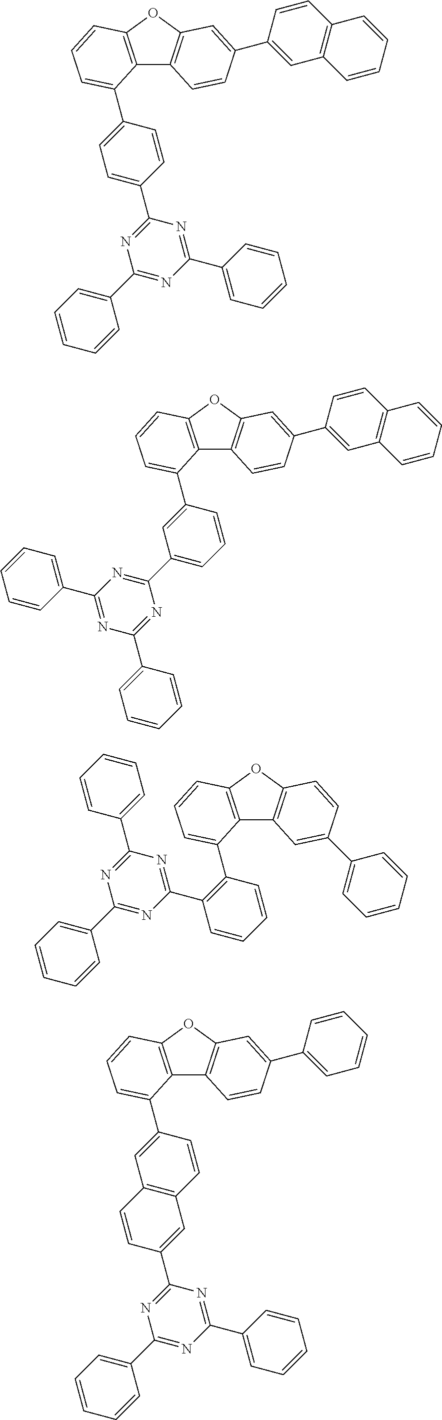

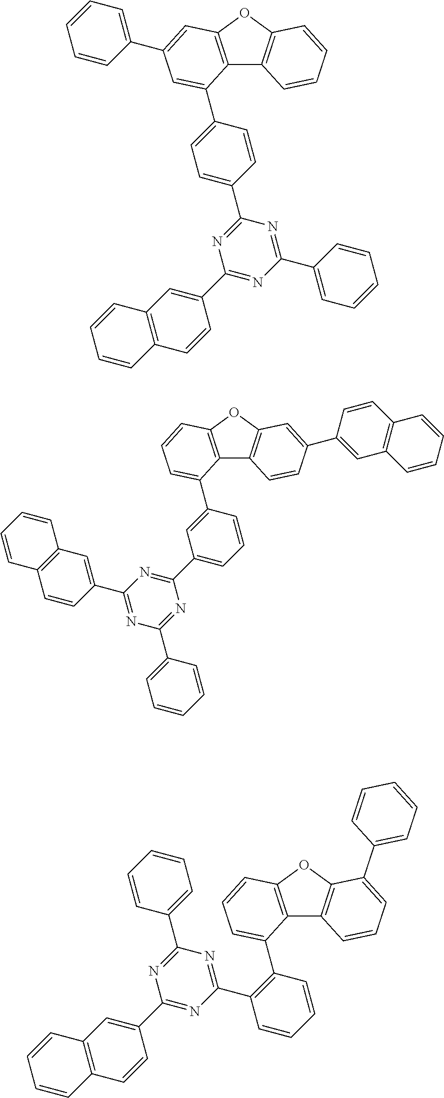 [SMILES: c1ccc(-c2cc(-c3ccc(-c4nc(-c5ccccc5)nc(-c5ccc6ccccc6c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4ccccc4c3)n2)cc1 ; c1ccc(-c2nc(-c3ccc4ccccc4c3)nc(-c3ccccc3-c3cccc4oc5c(-c6ccccc6)cccc5c34)n2)cc1]

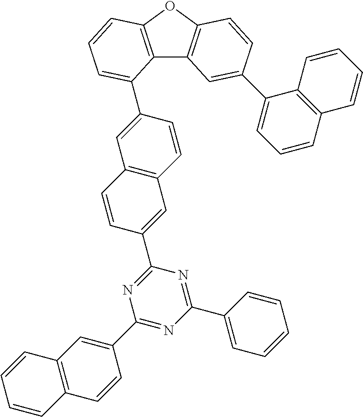 [SMILES: c1ccc(-c2nc(-c3ccc4ccccc4c3)nc(-c3ccc4cc(-c5cccc6oc7ccc(-c8cccc9ccccc89)cc7c56)ccc4c3)n2)cc1]

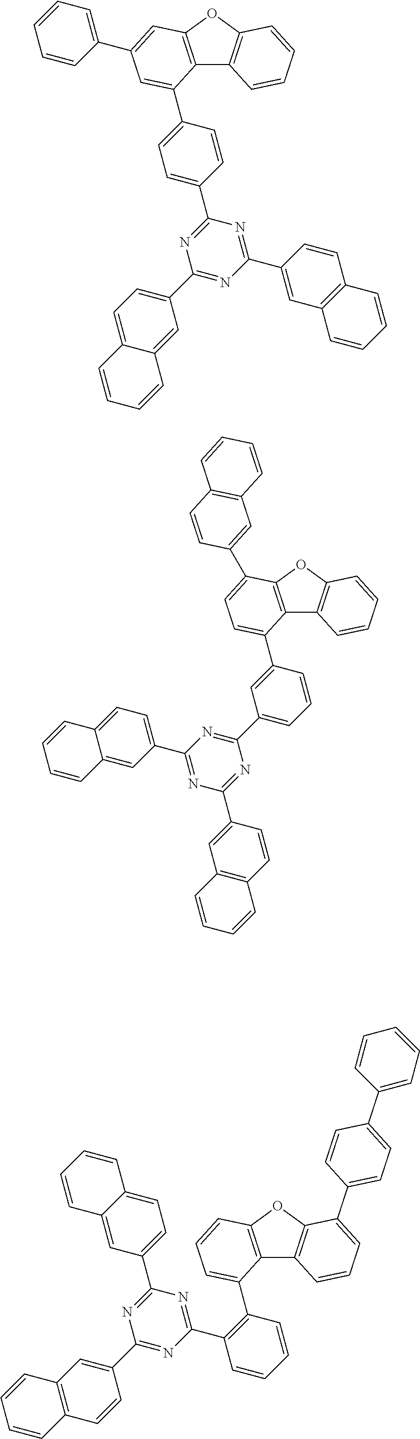 [SMILES: c1ccc(-c2cc(-c3ccc(-c4nc(-c5ccc6ccccc6c5)nc(-c5ccc6ccccc6c5)n4)cc3)c3c(c2)oc2ccccc23)cc1 ; c1cc(-c2nc(-c3ccc4ccccc4c3)nc(-c3ccc4ccccc4c3)n2)cc(-c2ccc(-c3ccc4ccccc4c3)c3oc4ccccc4c23)c1 ; c1ccc(-c2ccc(-c3cccc4c3oc3cccc(-c5ccccc5-c5nc(-c6ccc7ccccc7c6)nc(-c6ccc7ccccc7c6)n5)c34)cc2)cc1]

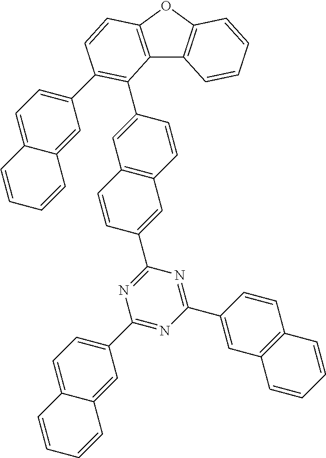 [SMILES: c1ccc2cc(-c3nc(-c4ccc5ccccc5c4)nc(-c4ccc5cc(-c6c(-c7ccc8ccccc8c7)ccc7oc8ccccc8c67)ccc5c4)n3)ccc2c1]

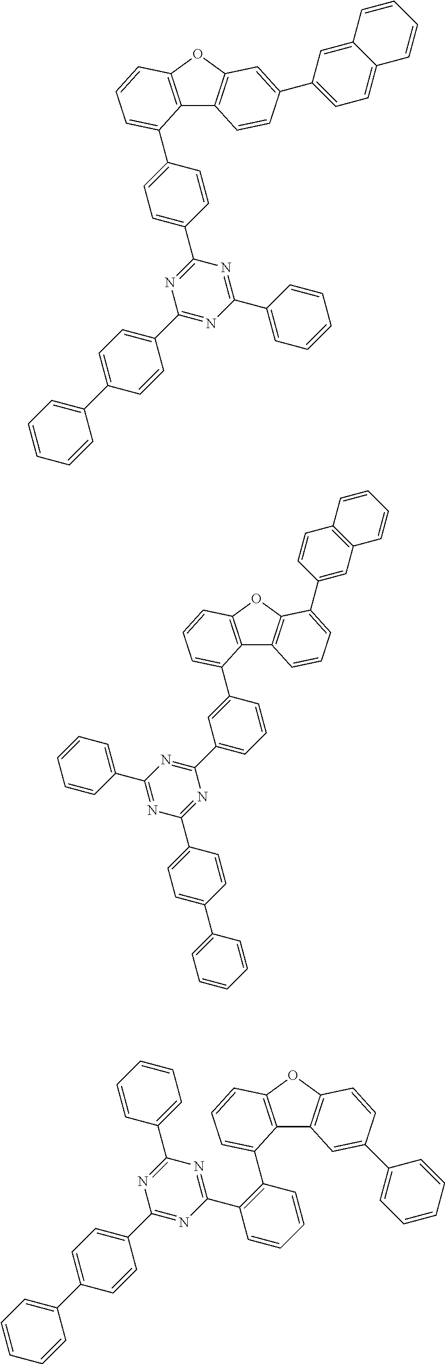 [SMILES: c1ccc(-c2ccc(-c3nc(-c4ccccc4)nc(-c4ccc(-c5cccc6oc7cc(-c8ccc9ccccc9c8)ccc7c56)cc4)n3)cc2)cc1 ; c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2ccc(-c3nc(-c4ccccc4)nc(-c4ccccc4-c4cccc5oc6ccc(-c7ccccc7)cc6c45)n3)cc2)cc1]

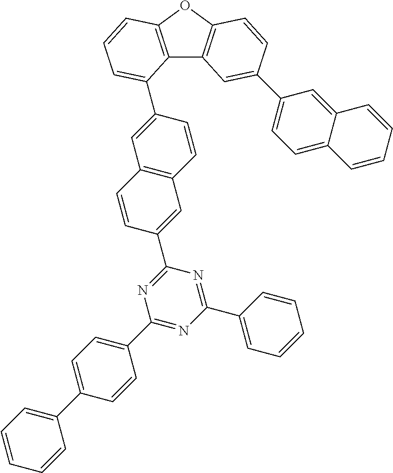 [SMILES: c1ccc(-c2ccc(-c3nc(-c4ccccc4)nc(-c4ccc5cc(-c6cccc7oc8ccc(-c9ccc%10ccccc%10c9)cc8c67)ccc5c4)n3)cc2)cc1]

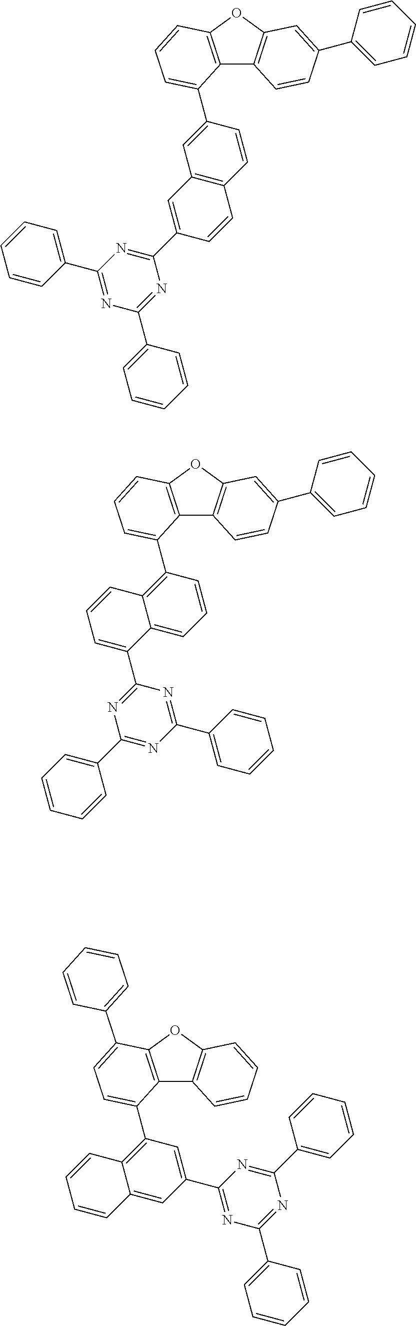 [SMILES: c1ccc(-c2ccc3c(c2)oc2cccc(-c4ccc5ccc(-c6nc(-c7ccccc7)nc(-c7ccccc7)n6)cc5c4)c23)cc1 ; c1ccc(-c2ccc3c(c2)oc2cccc(-c4cccc5c(-c6nc(-c7ccccc7)nc(-c7ccccc7)n6)cccc45)c23)cc1 ; c1ccc(-c2nc(-c3ccccc3)nc(-c3cc(-c4ccc(-c5ccccc5)c5oc6ccccc6c45)c4ccccc4c3)n2)cc1]

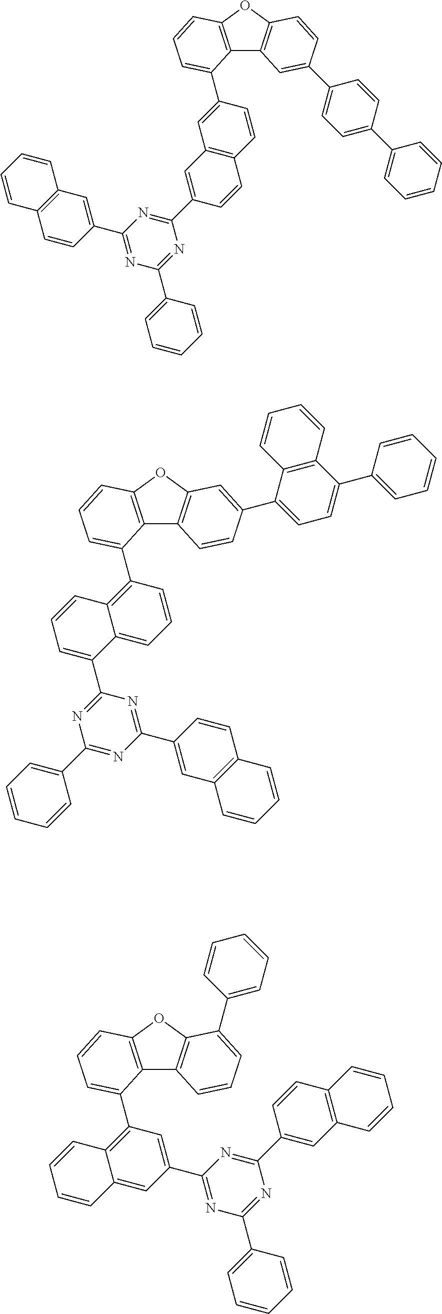 [SMILES: c1ccc(-c2ccc(-c3ccc4oc5cccc(-c6ccc7ccc(-c8nc(-c9ccccc9)nc(-c9ccc%10ccccc%10c9)n8)cc7c6)c5c4c3)cc2)cc1 ; c1ccc(-c2nc(-c3ccc4ccccc4c3)nc(-c3cccc4c(-c5cccc6oc7cc(-c8ccc(-c9ccccc9)c9ccccc89)ccc7c56)cccc34)n2)cc1 ; c1ccc(-c2nc(-c3ccc4ccccc4c3)nc(-c3cc(-c4cccc5oc6c(-c7ccccc7)cccc6c45)c4ccccc4c3)n2)cc1]

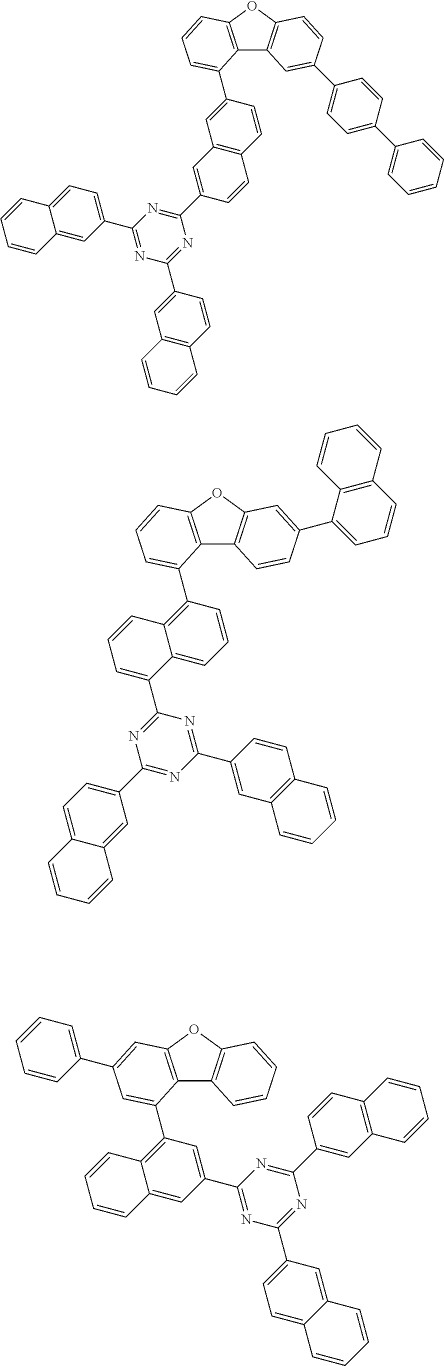 [SMILES: c1ccc(-c2ccc(-c3ccc4oc5cccc(-c6ccc7ccc(-c8nc(-c9ccc%10ccccc%10c9)nc(-c9ccc%10ccccc%10c9)n8)cc7c6)c5c4c3)cc2)cc1 ; c1ccc2cc(-c3nc(-c4ccc5ccccc5c4)nc(-c4cccc5c(-c6cccc7oc8cc(-c9cccc%10ccccc9%10)ccc8c67)cccc45)n3)ccc2c1 ; c1ccc(-c2cc(-c3cc(-c4nc(-c5ccc6ccccc6c5)nc(-c5ccc6ccccc6c5)n4)cc4ccccc34)c3c(c2)oc2ccccc23)cc1]

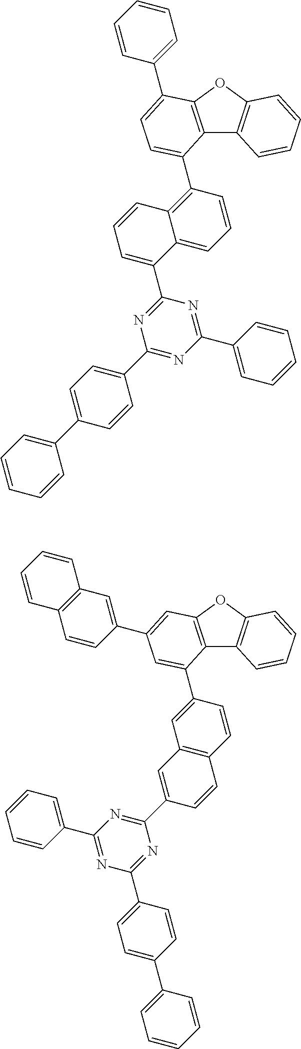 [SMILES: c1ccc(-c2ccc(-c3nc(-c4ccccc4)nc(-c4cccc5c(-c6ccc(-c7ccccc7)c7oc8ccccc8c67)cccc45)n3)cc2)cc1 ; c1ccc(-c2ccc(-c3nc(-c4ccccc4)nc(-c4ccc5ccc(-c6cc(-c7ccc8ccccc8c7)cc7oc8ccccc8c67)cc5c4)n3)cc2)cc1]

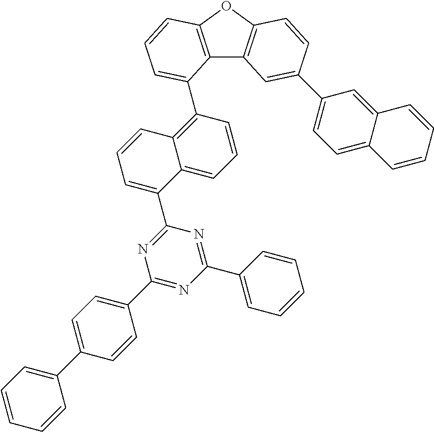 [SMILES: c1ccc(-c2ccc(-c3nc(-c4ccccc4)nc(-c4cccc5c(-c6cccc7oc8ccc(-c9ccc%10ccccc%10c9)cc8c67)cccc45)n3)cc2)cc1]

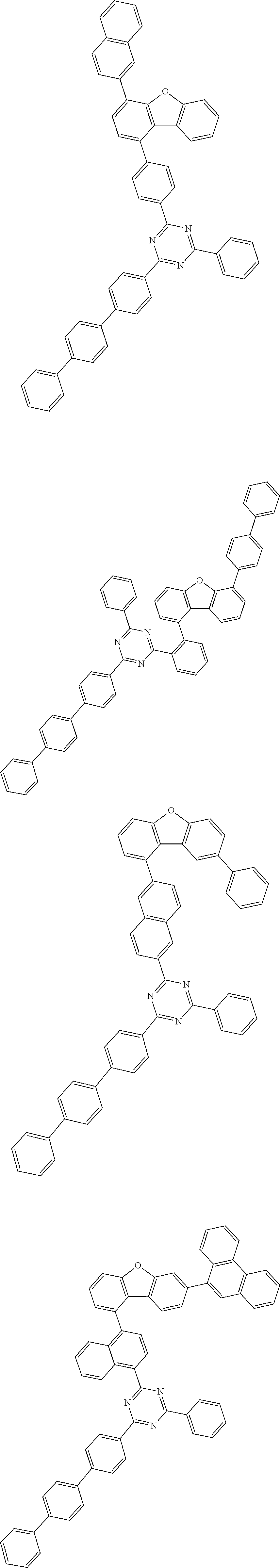 [SMILES: c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1 ; c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1]

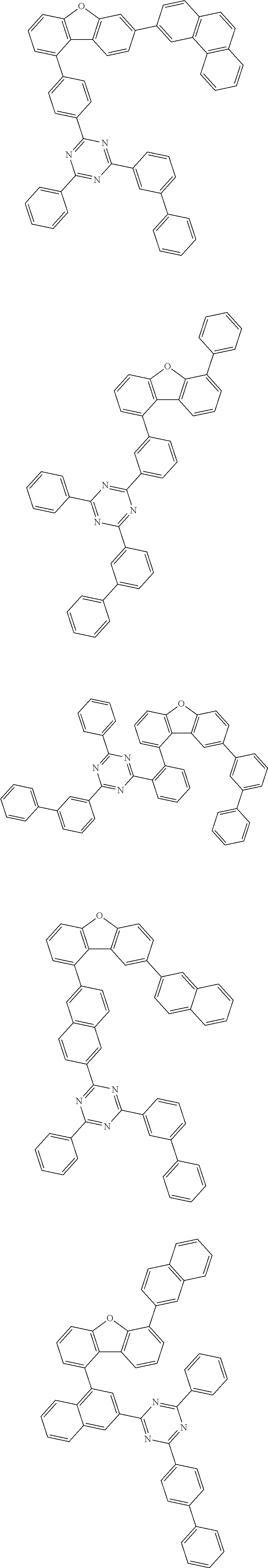 [SMILES: c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1 ; c1ccc(-c2cccc(-c3nc(-c4ccccc4)nc(-c4cccc(-c5cccc6oc7c(-c8ccccc8)cccc7c56)c4)n3)c2)cc1 ; c1ccc(-c2cccc(-c3ccc4oc5cccc(-c6ccccc6-c6nc(-c7ccccc7)nc(-c7cccc(-c8ccccc8)c7)n6)c5c4c3)c2)cc1 ; c1ccc(-c2cccc(-c3nc(-c4ccccc4)nc(-c4ccc5cc(-c6cccc7oc8ccc(-c9ccc%10ccccc%10c9)cc8c67)ccc5c4)n3)c2)cc1 ; c1ccc(-c2ccc(-c3nc(-c4ccccc4)nc(-c4cc(-c5cccc6oc7c(-c8ccc9ccccc9c8)cccc7c56)c5ccccc5c4)n3)cc2)cc1]

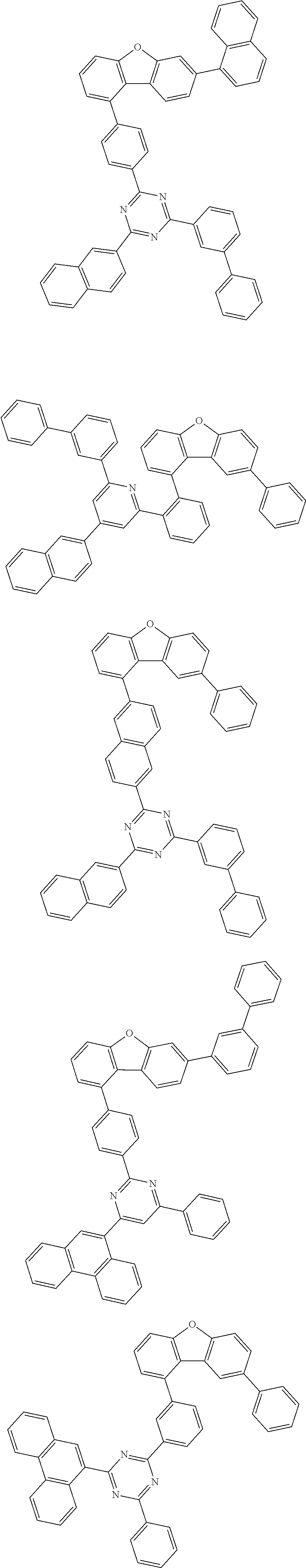

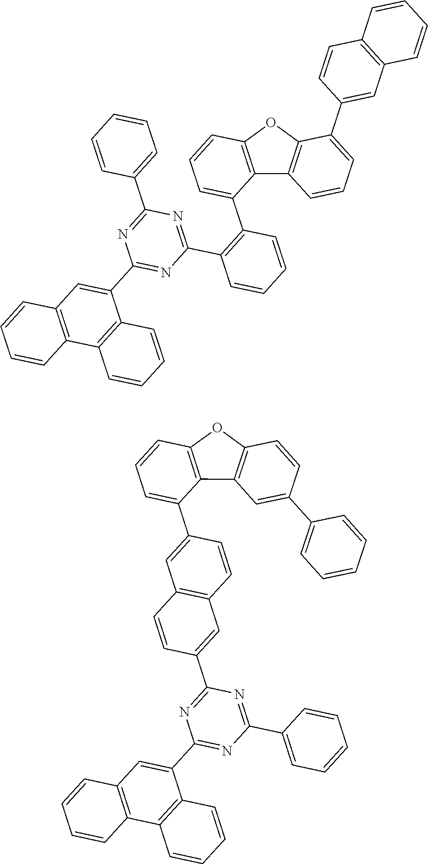 [SMILES: c1ccc(-c2nc(-c3ccccc3-c3cccc4oc5c(-c6ccc7ccccc7c6)cccc5c34)nc(-c3cc4ccccc4c4ccccc34)n2)cc1 ; c1ccc(-c2ccc3oc4cccc(-c5ccc6cc(-c7nc(-c8ccccc8)nc(-c8cc9ccccc9c9ccccc89)n7)ccc6c5)c4c3c2)cc1]

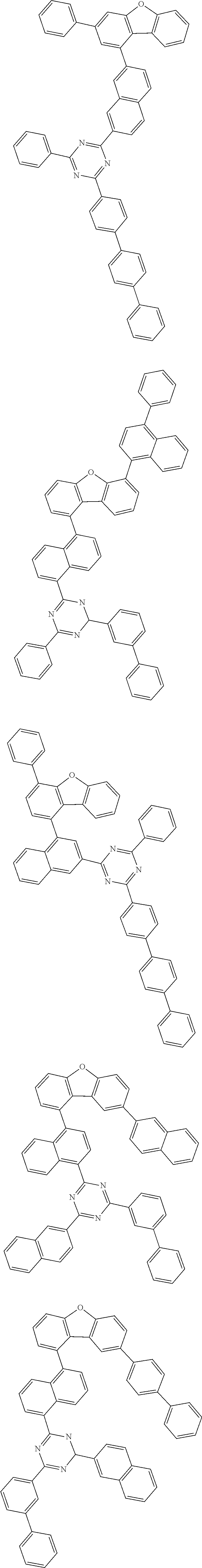

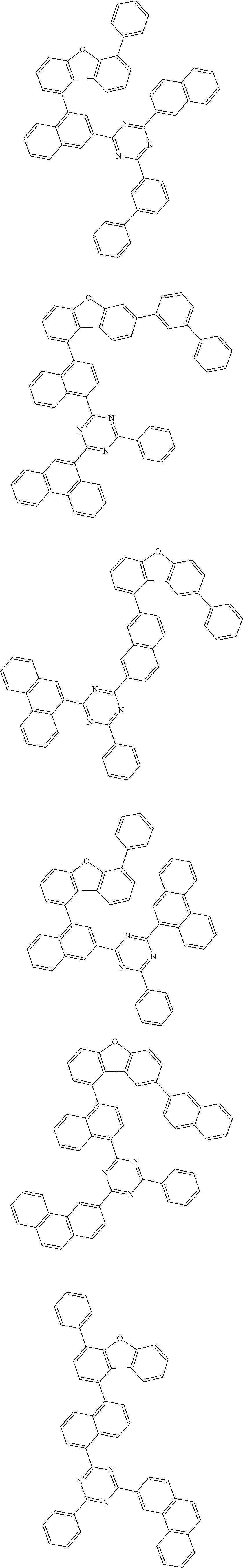

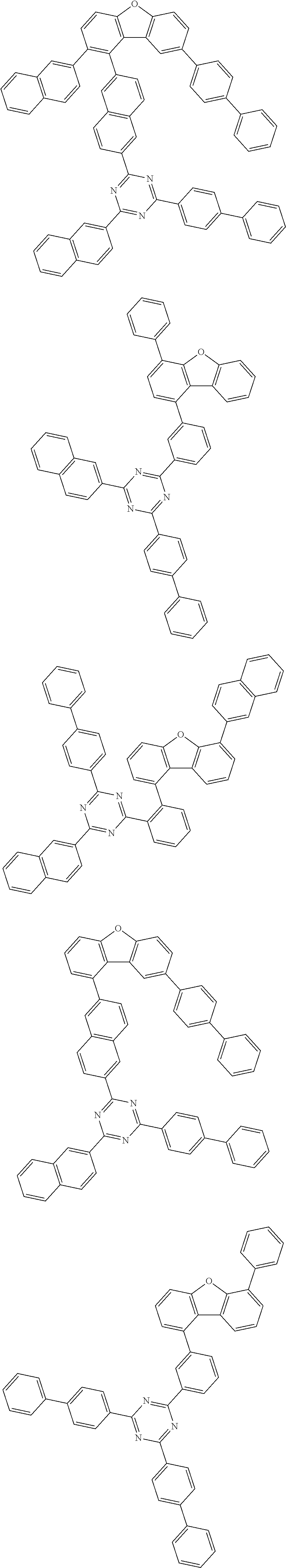

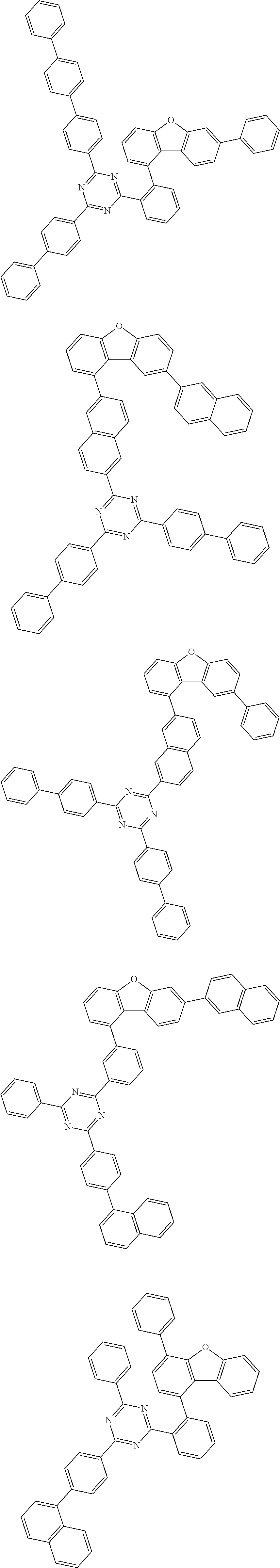

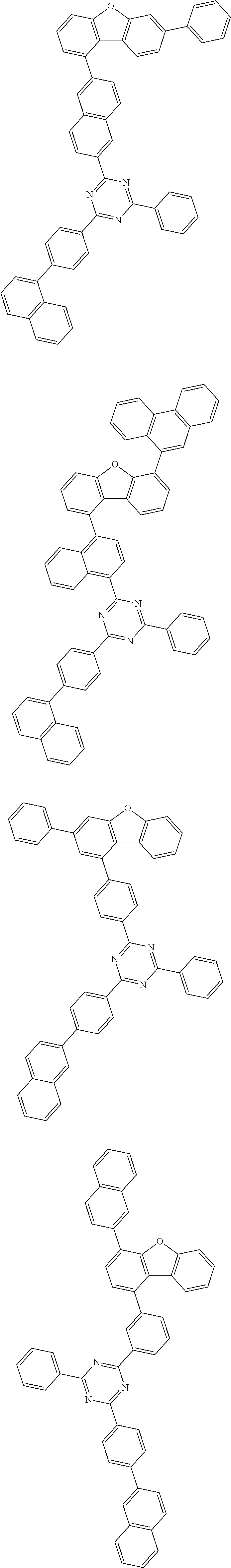 [SMILES: c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1 ; c1ccc(-c2nc(-c3ccc(-c4cccc5ccccc45)cc3)nc(-c3ccc(-c4cccc5oc6c(-c7cc8ccccc8c8ccccc78)cccc6c45)c4ccccc34)n2)cc1 ; c1ccc(-c2cc(-c3ccc(-c4nc(-c5ccccc5)nc(-c5ccc(-c6ccc7ccccc7c6)cc5)n4)cc3)c3c(c2)oc2ccccc23)cc1 ; c1ccc(-c2nc(-c3ccc(-c4ccc5ccccc5c4)cc3)nc(-c3cccc(-c4ccc(-c5ccc6ccccc6c5)c5oc6ccccc6c45)c3)n2)cc1]

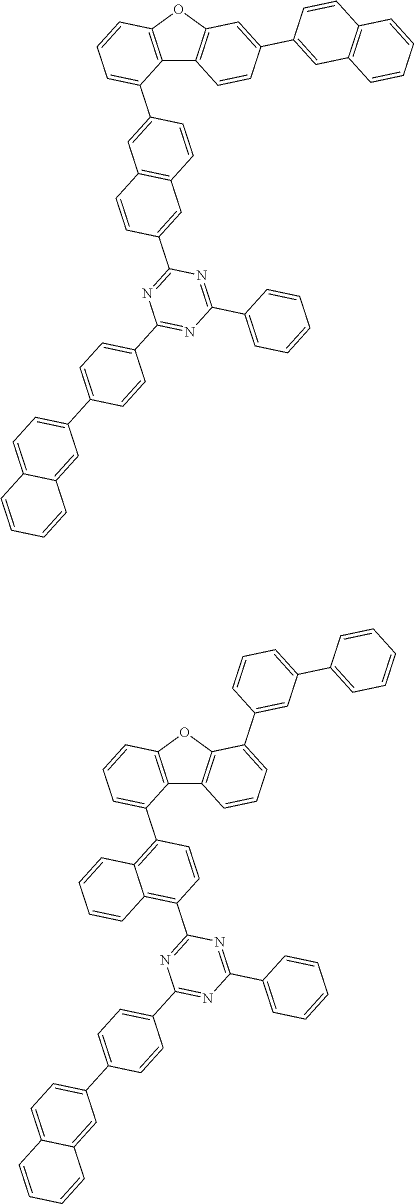 [SMILES: c1ccc(-c2nc(-c3ccc(-c4ccc5ccccc5c4)cc3)nc(-c3ccc4cc(-c5cccc6oc7cc(-c8ccc9ccccc9c8)ccc7c56)ccc4c3)n2)cc1 ; c1ccc(-c2cccc(-c3cccc4c3oc3cccc(-c5ccc(-c6nc(-c7ccccc7)nc(-c7ccc(-c8ccc9ccccc9c8)cc7)n6)c6ccccc56)c34)c2)cc1]

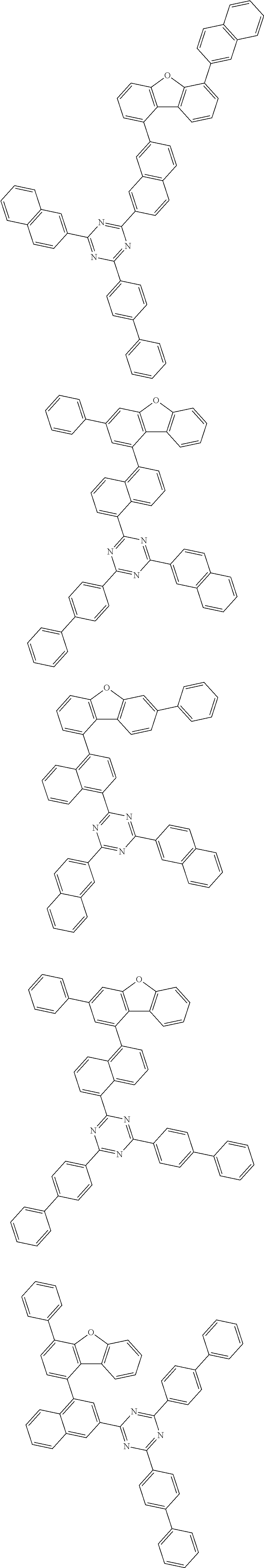

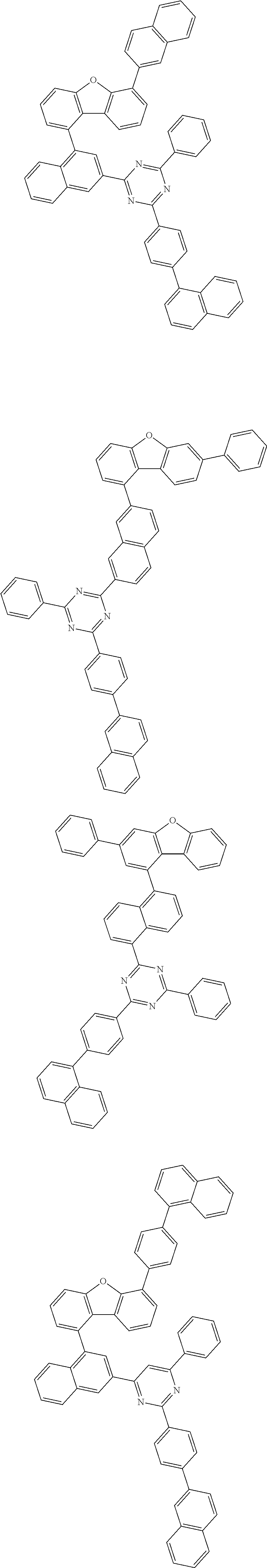 [SMILES: c1ccc(-c2nc(-c3ccc(-c4cccc5ccccc45)cc3)nc(-c3cc(-c4cccc5oc6c(-c7ccc8ccccc8c7)cccc6c45)c4ccccc4c3)n2)cc1 ; c1ccc(-c2ccc3c(c2)oc2cccc(-c4ccc5ccc(-c6nc(-c7ccccc7)nc(-c7ccc(-c8ccc9ccccc9c8)cc7)n6)cc5c4)c23)cc1 ; c1ccc(-c2cc(-c3cccc4c(-c5nc(-c6ccccc6)nc(-c6ccc(-c7cccc8ccccc78)cc6)n5)cccc34)c3c(c2)oc2ccccc23)cc1 ; c1ccc(-c2cc(-c3cc(-c4cccc5oc6c(-c7ccc(-c8cccc9ccccc89)cc7)cccc6c45)c4ccccc4c3)nc(-c3ccc(-c4ccc5ccccc5c4)cc3)n2)cc1]

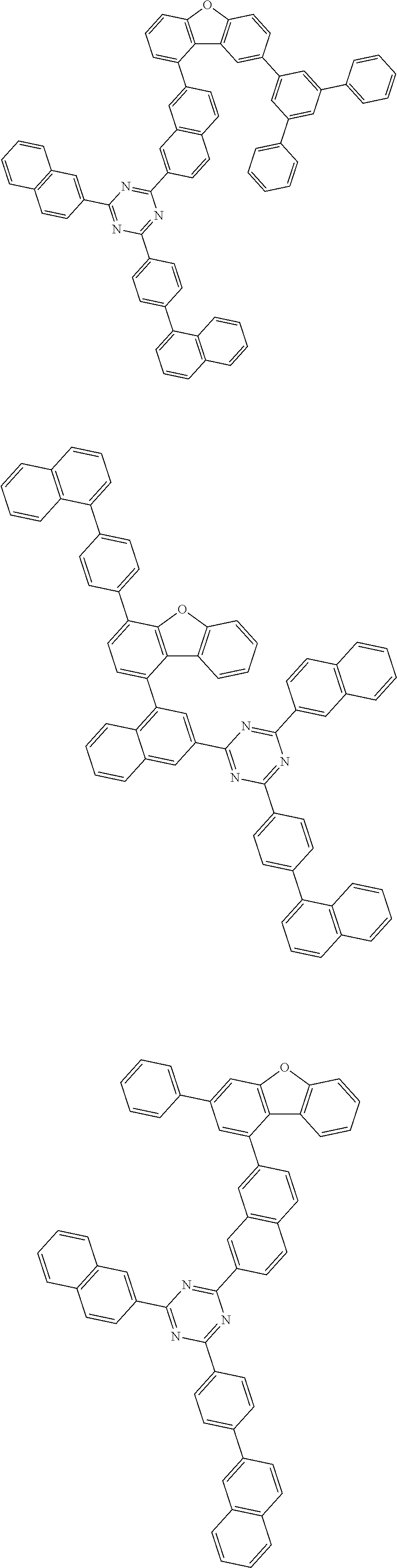 [SMILES: c1ccc(-c2cc(-c3ccccc3)cc(-c3ccc4oc5cccc(-c6ccc7ccc(-c8nc(-c9ccc(-c%10cccc%11ccccc%10%11)cc9)nc(-c9ccc%10ccccc%10c9)n8)cc7c6)c5c4c3)c2)cc1 ; c1ccc2cc(-c3nc(-c4ccc(-c5cccc6ccccc56)cc4)nc(-c4cc(-c5ccc(-c6ccc(-c7cccc8ccccc78)cc6)c6oc7ccccc7c56)c5ccccc5c4)n3)ccc2c1 ; c1ccc(-c2cc(-c3ccc4ccc(-c5nc(-c6ccc(-c7ccc8ccccc8c7)cc6)nc(-c6ccc7ccccc7c6)n5)cc4c3)c3c(c2)oc2ccccc23)cc1]

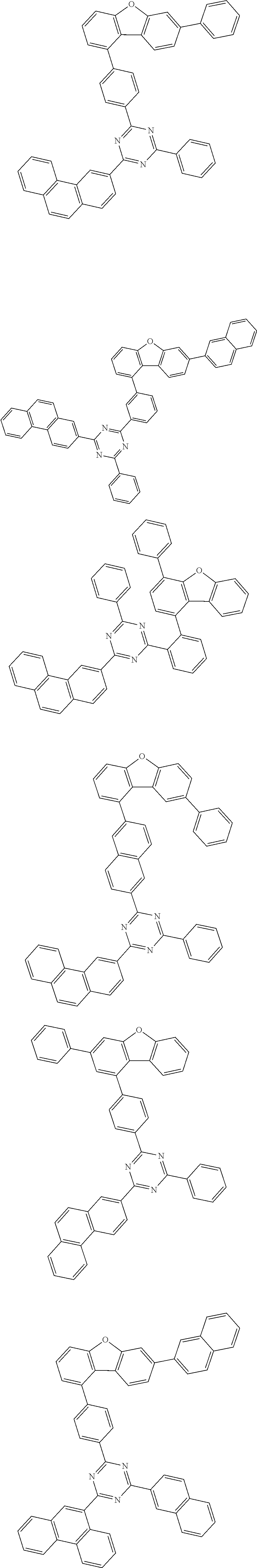

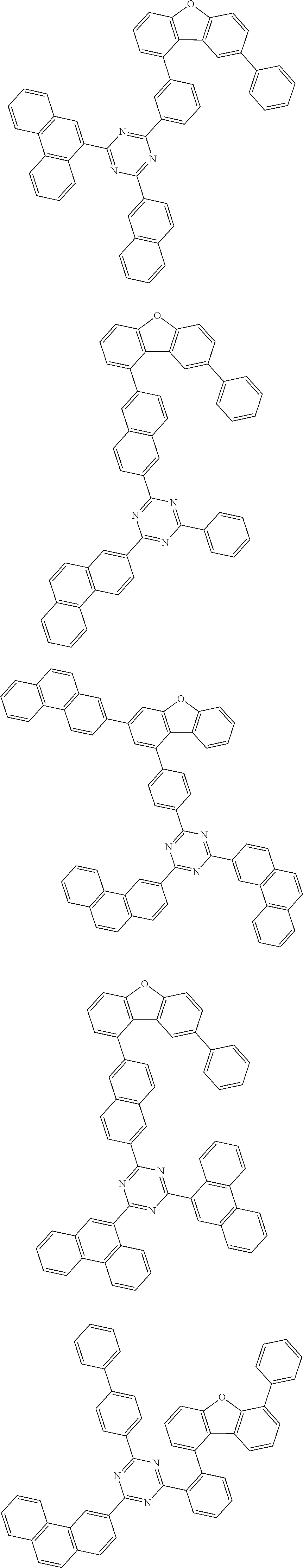

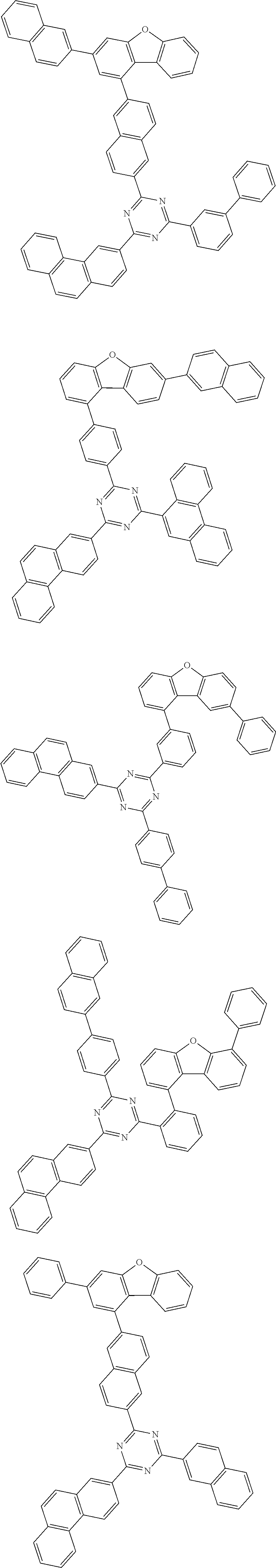

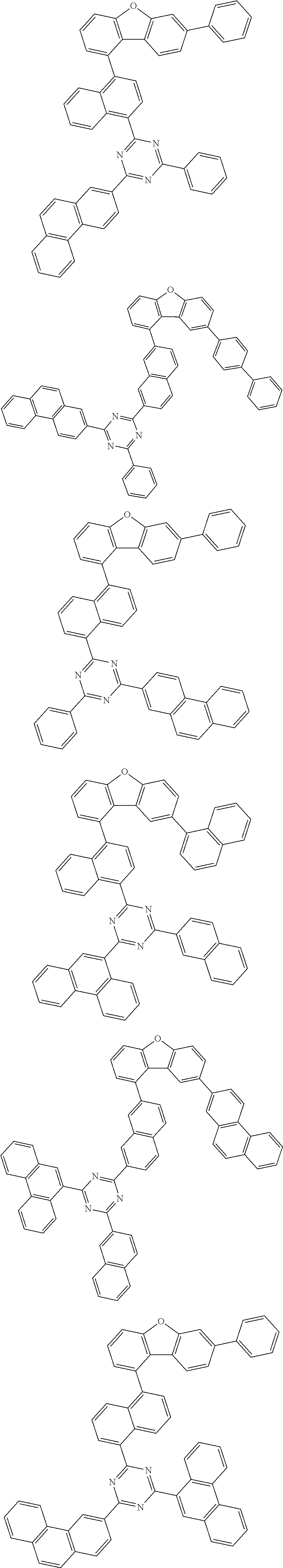

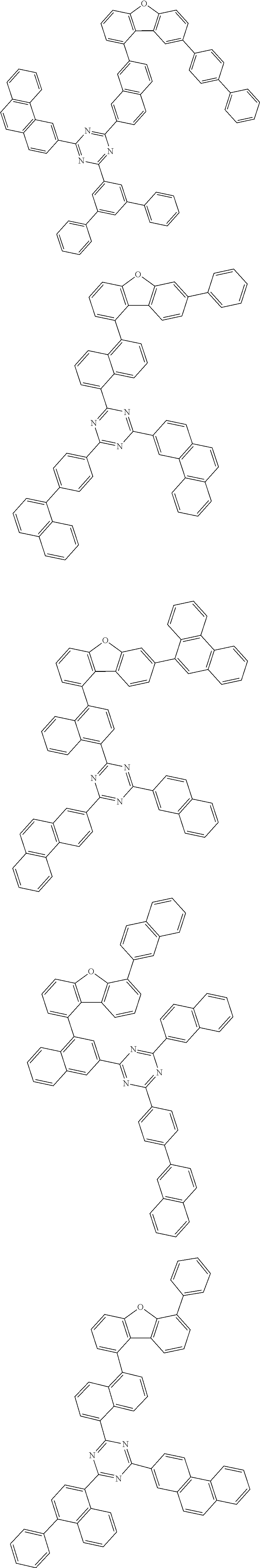

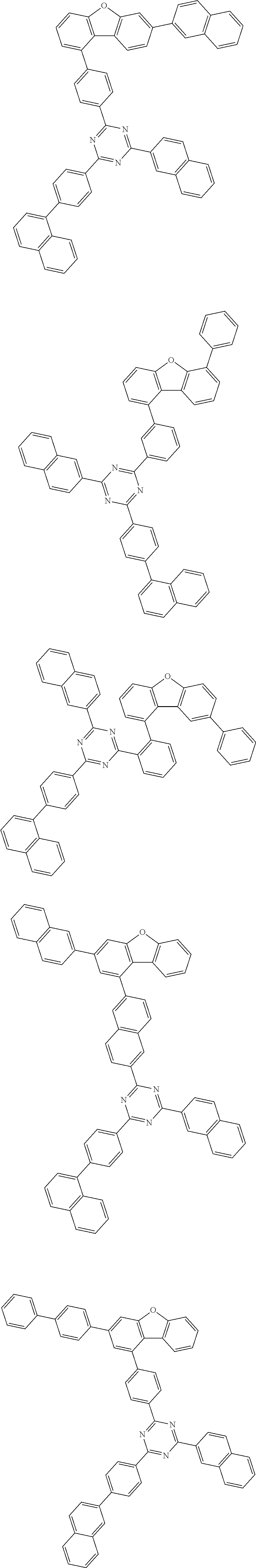

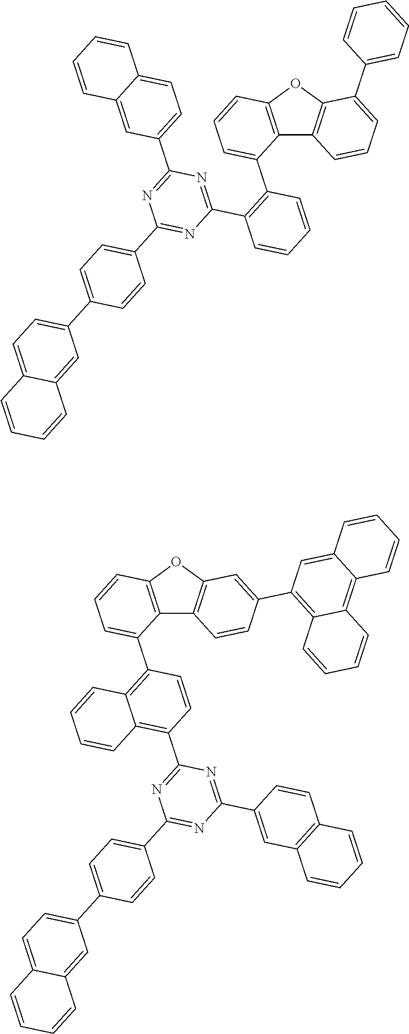 [SMILES: c1ccc(-c2cccc3c2oc2cccc(-c4ccccc4-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1]

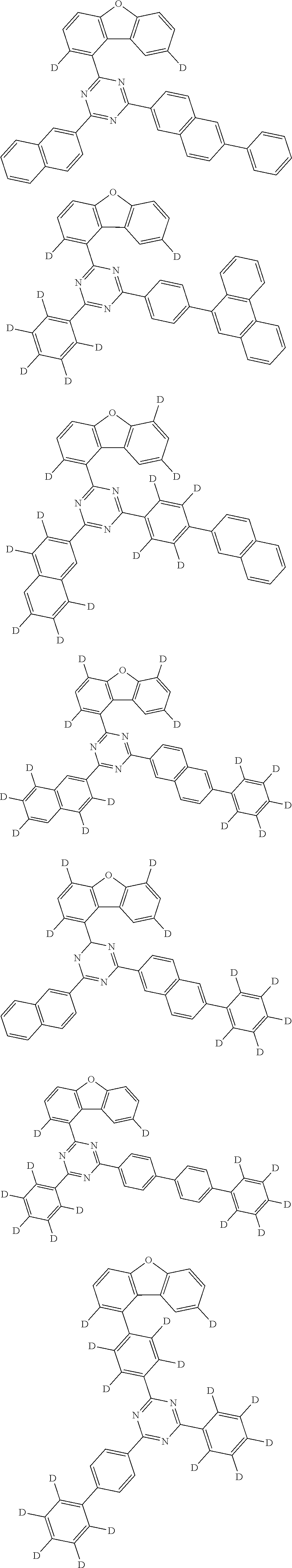

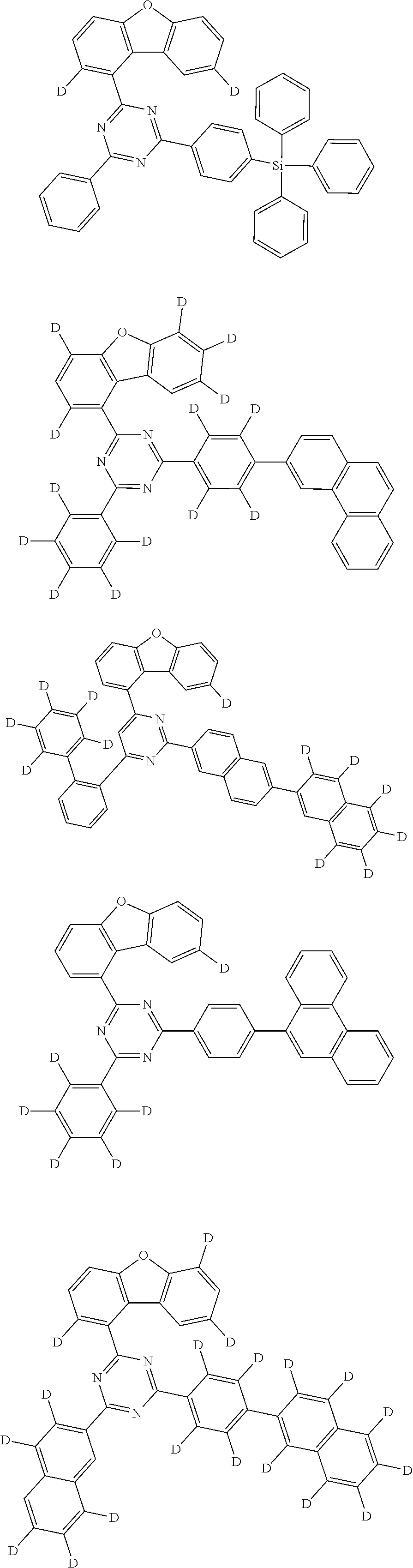

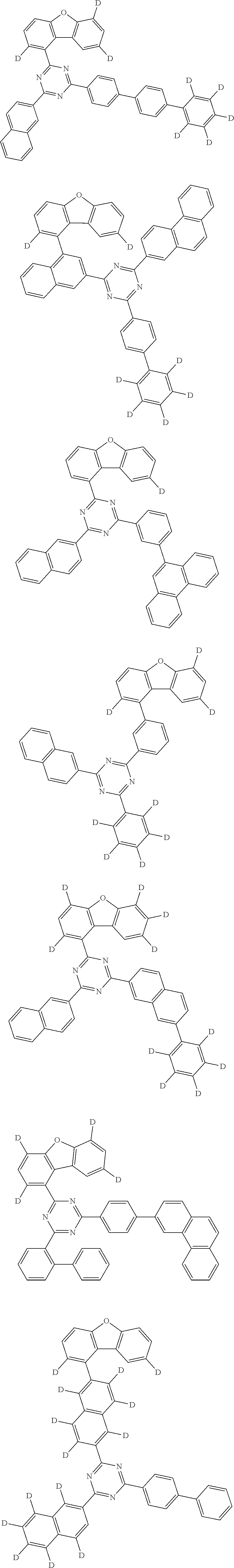

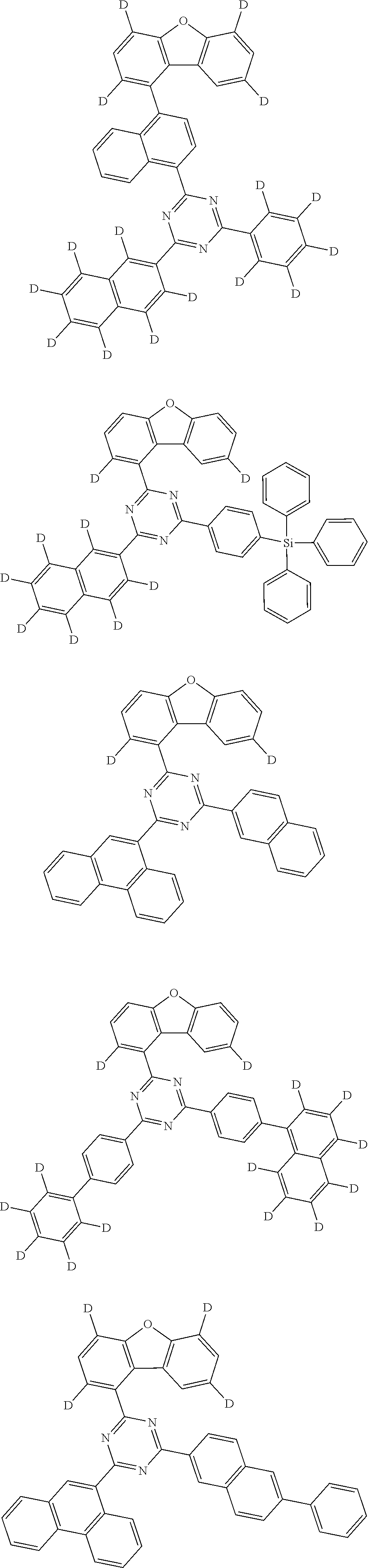

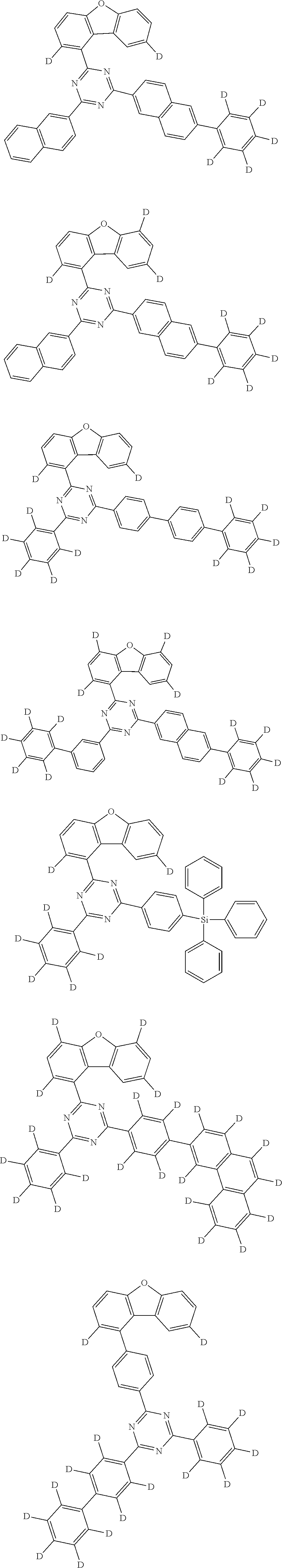

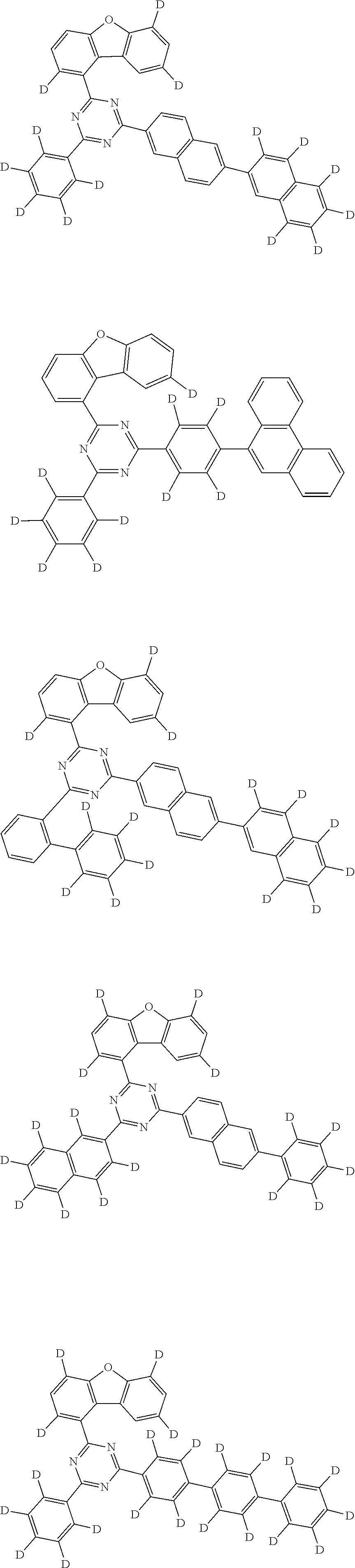

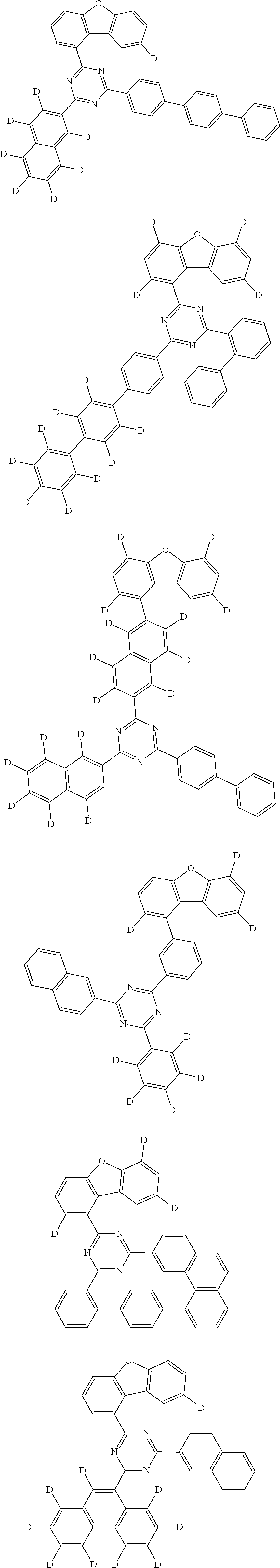

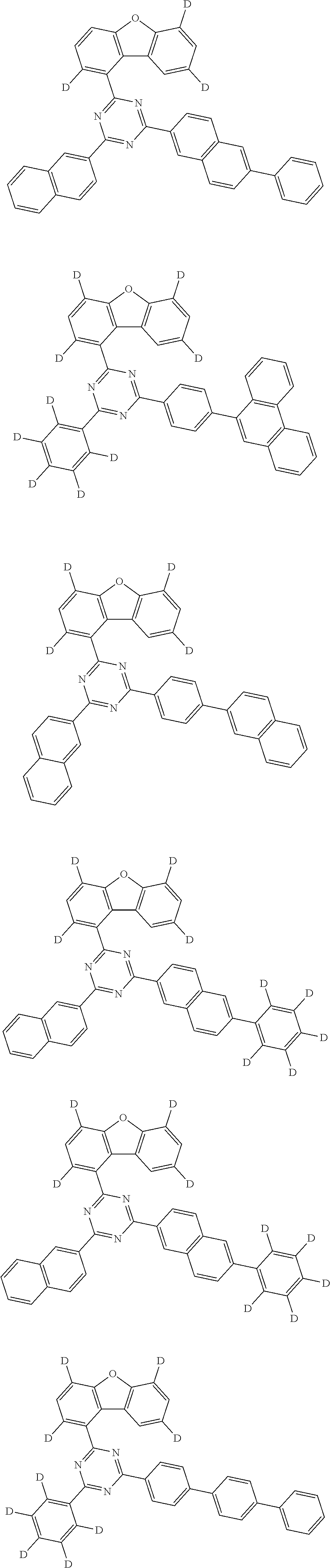

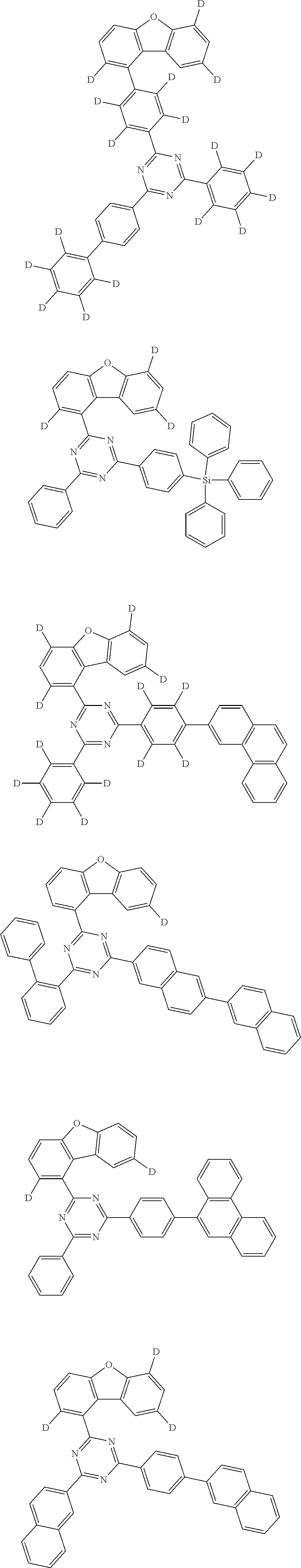

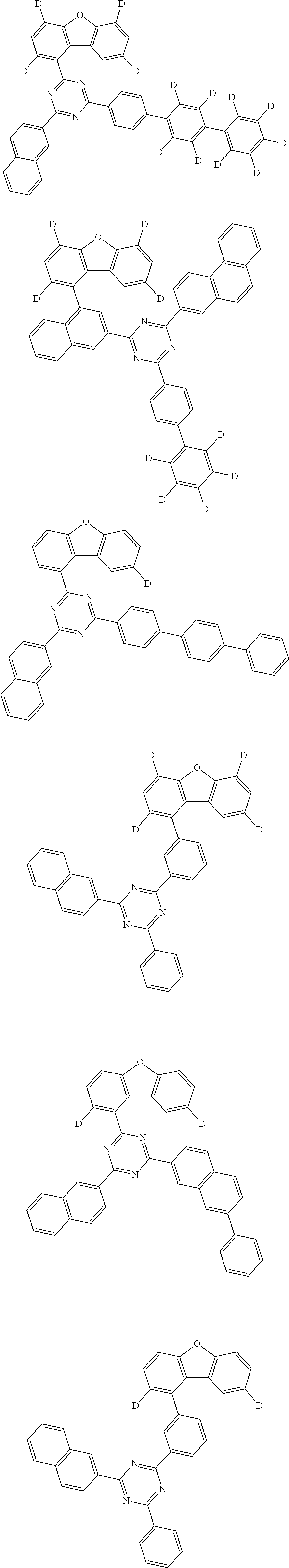

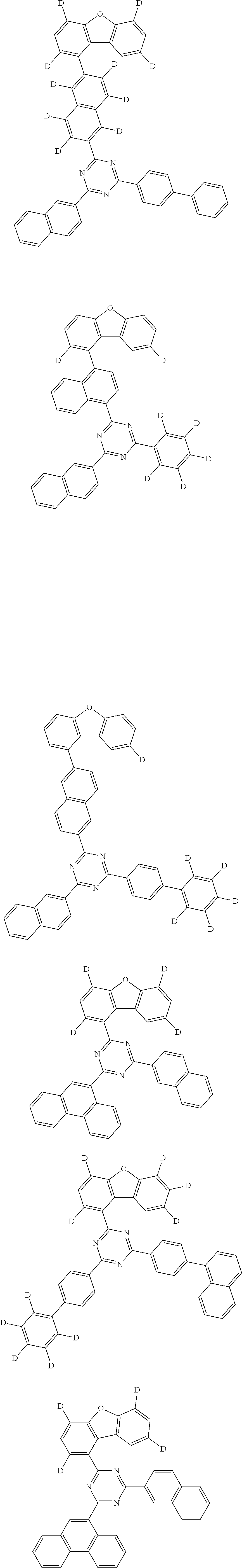

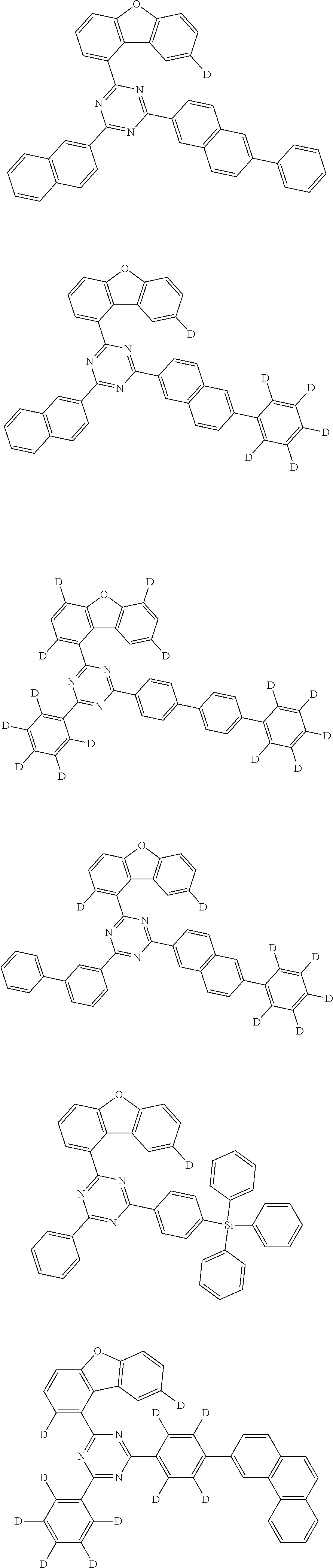

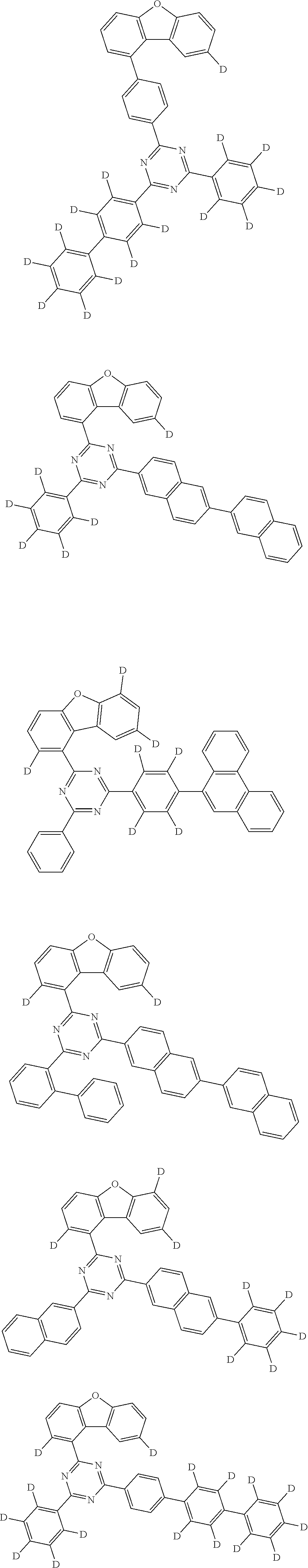

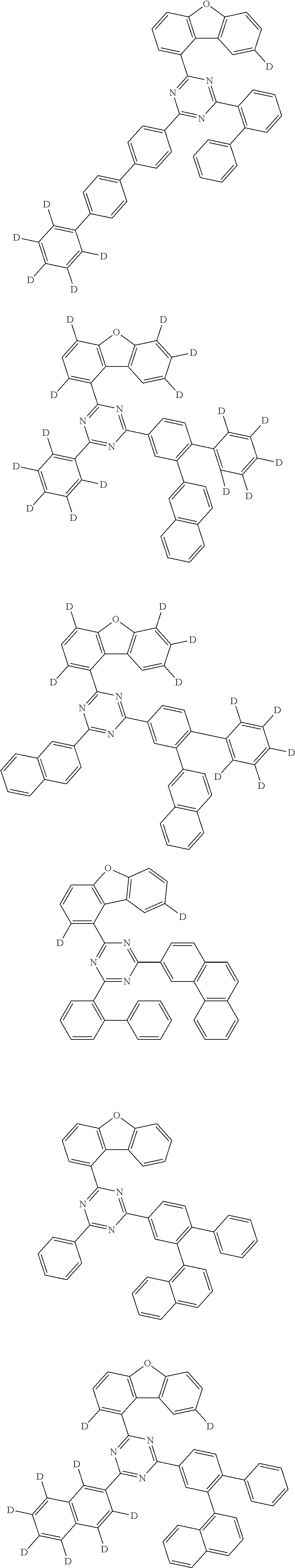

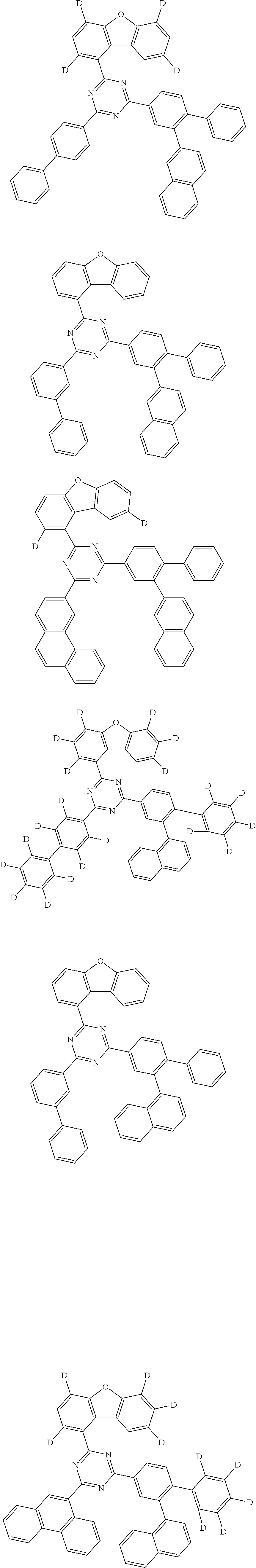

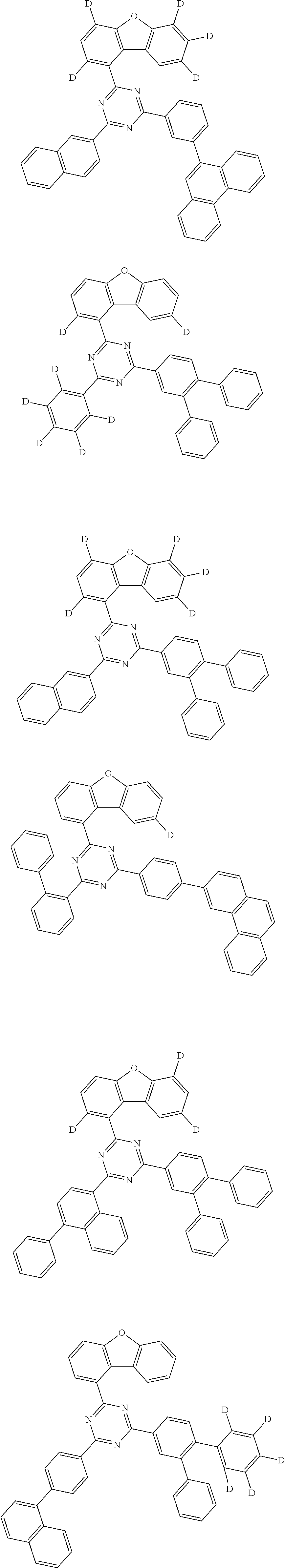

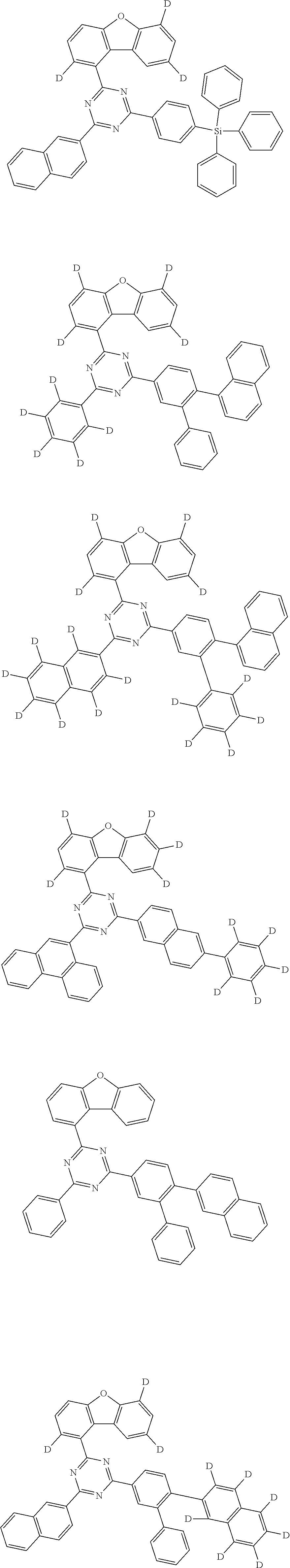

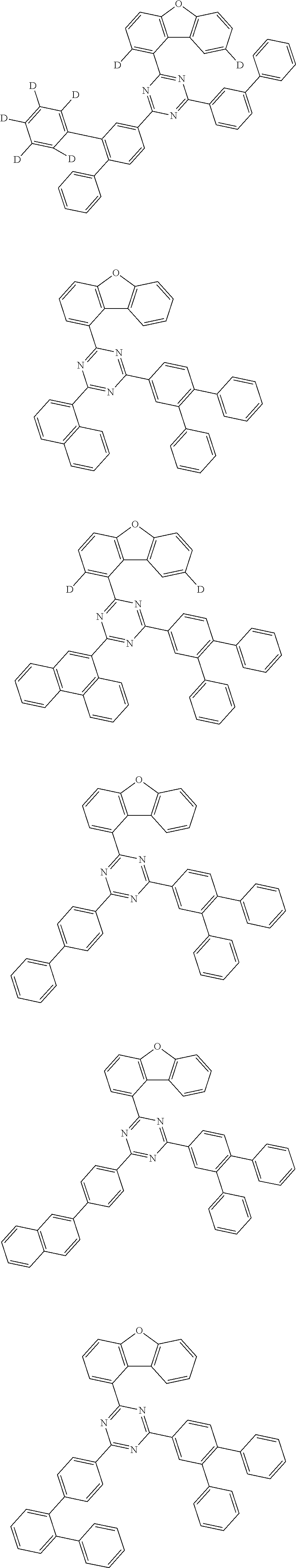

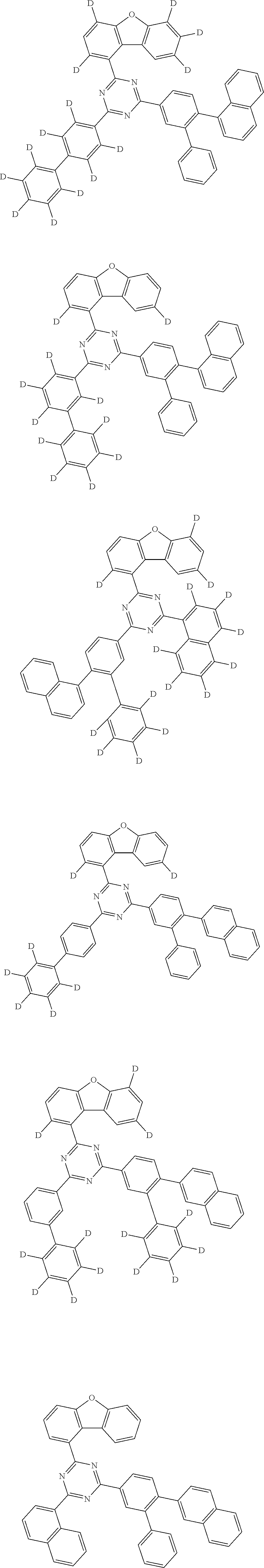

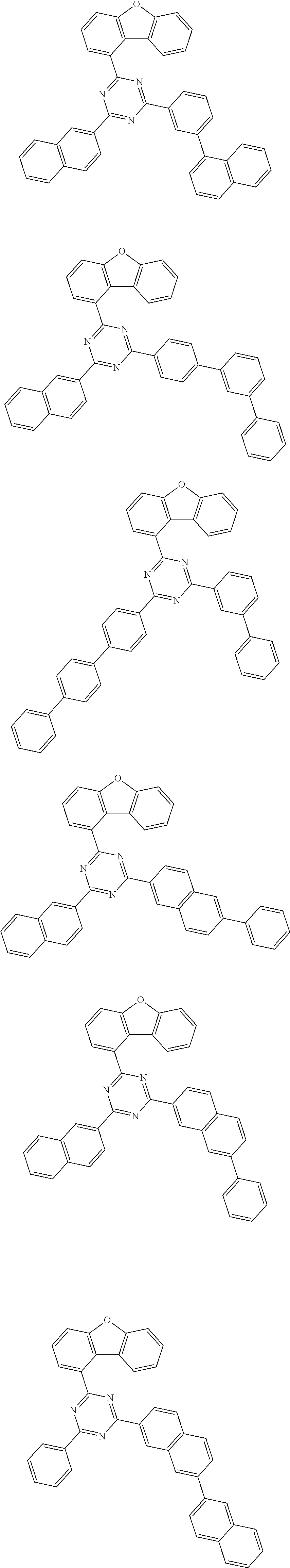 [SMILES: c1cc(-c2nc(-c3ccc4ccccc4c3)nc(-c3cccc4oc5ccccc5c34)n2)cc(-c2cccc3ccccc23)c1 ; c1ccc(-c2cccc(-c3ccc(-c4nc(-c5ccc6ccccc6c5)nc(-c5cccc6oc7ccccc7c56)n4)cc3)c2)cc1 ; c1ccc(-c2ccc(-c3ccc(-c4nc(-c5cccc(-c6ccccc6)c5)nc(-c5cccc6oc7ccccc7c56)n4)cc3)cc2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2nc(-c3ccc4ccc(-c5ccc6ccccc6c5)cc4c3)nc(-c3cccc4oc5ccccc5c34)n2)cc1]

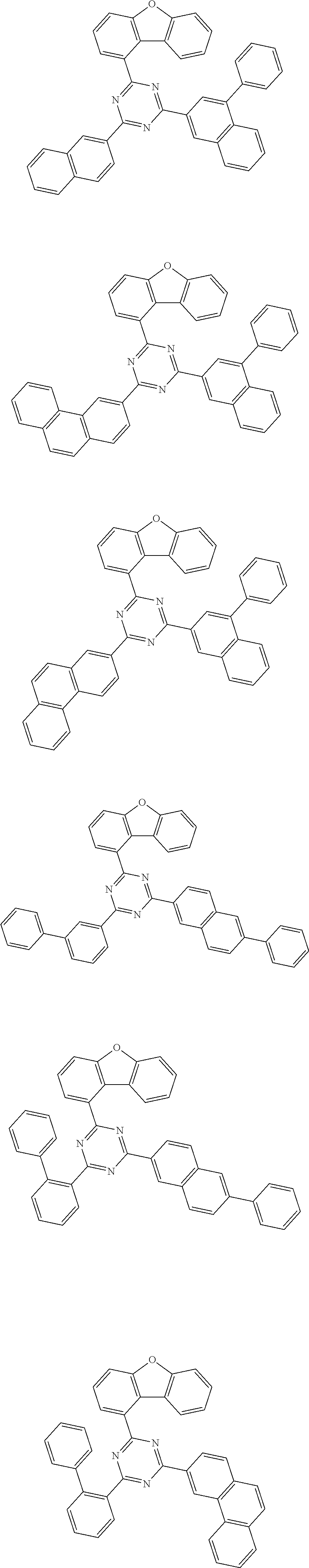 [SMILES: c1ccc(-c2cc(-c3nc(-c4ccc5ccccc5c4)nc(-c4cccc5oc6ccccc6c45)n3)cc3ccccc23)cc1 ; c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc3cc(-c4nc(-c5ccccc5-c5ccccc5)nc(-c5cccc6oc7ccccc7c56)n4)ccc3c2)cc1 ; c1ccc(-c2ccccc2-c2nc(-c3ccc4ccc5ccccc5c4c3)nc(-c3cccc4oc5ccccc5c34)n2)cc1]

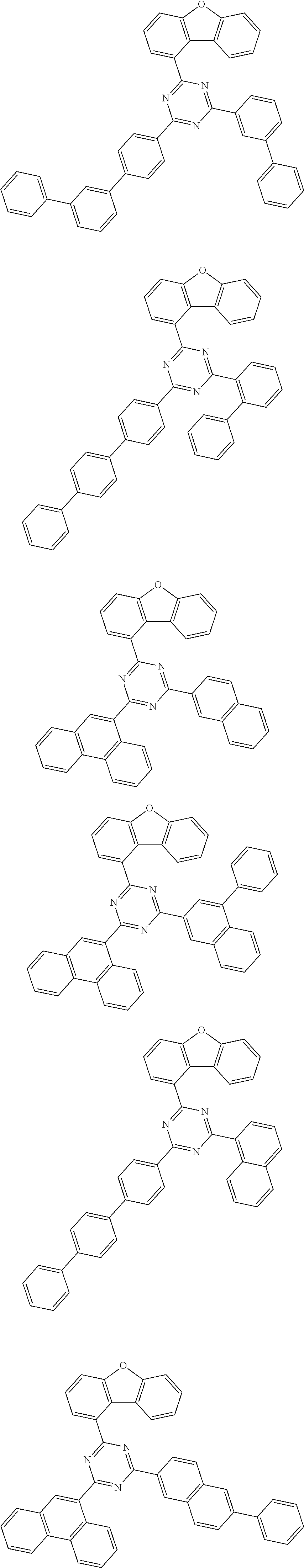

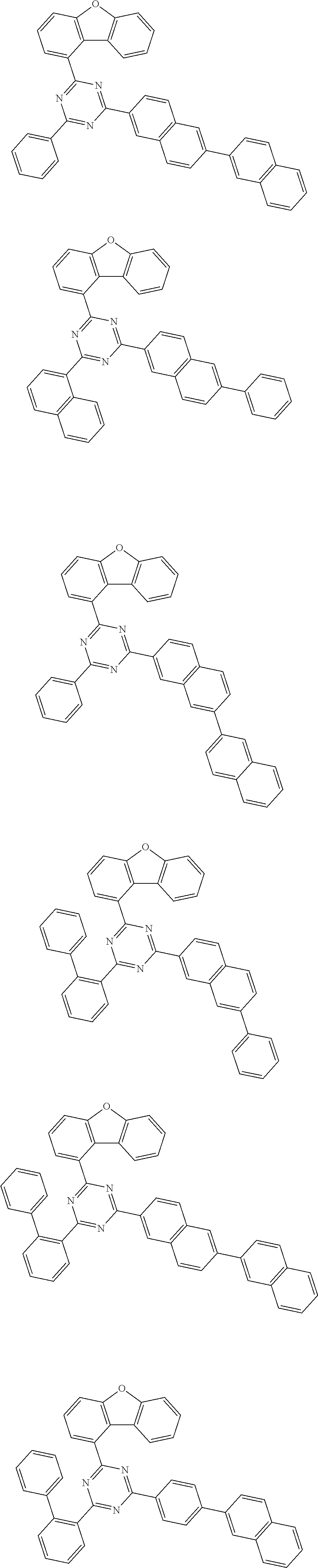 [SMILES: c1ccc(-c2nc(-c3ccc4cc(-c5ccc6ccccc6c5)ccc4c3)nc(-c3cccc4oc5ccccc5c34)n2)cc1 ; c1ccc(-c2ccc3cc(-c4nc(-c5cccc6ccccc56)nc(-c5cccc6oc7ccccc7c56)n4)ccc3c2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3ccc(-c4nc(-c5ccccc5-c5ccccc5)nc(-c5cccc6oc7ccccc7c56)n4)cc3c2)cc1 ; c1ccc(-c2ccccc2-c2nc(-c3ccc4cc(-c5ccc6ccccc6c5)ccc4c3)nc(-c3cccc4oc5ccccc5c34)n2)cc1 ; c1ccc(-c2ccccc2-c2nc(-c3ccc(-c4ccc5ccccc5c4)cc3)nc(-c3cccc4oc5ccccc5c34)n2)cc1]

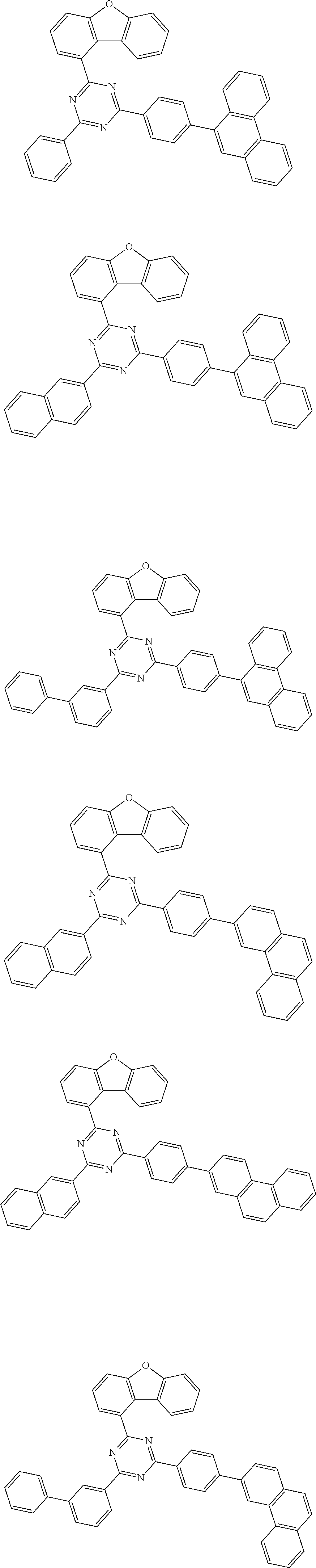

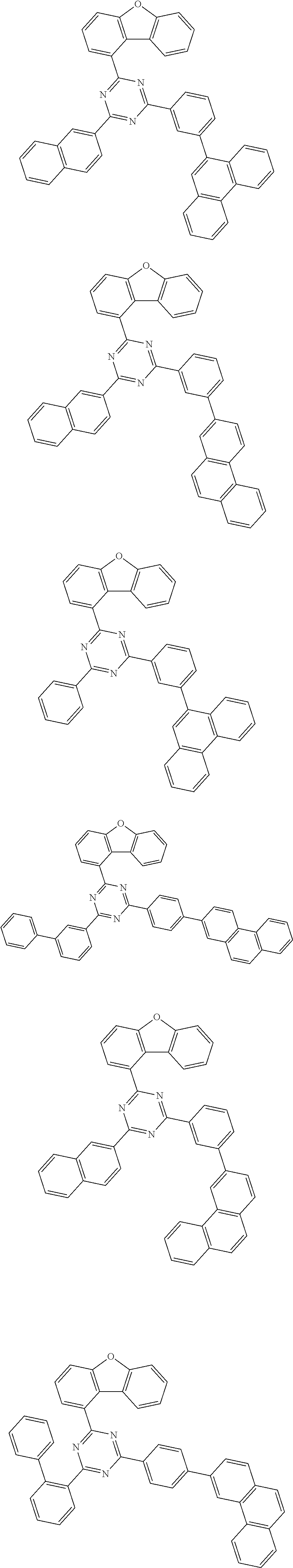

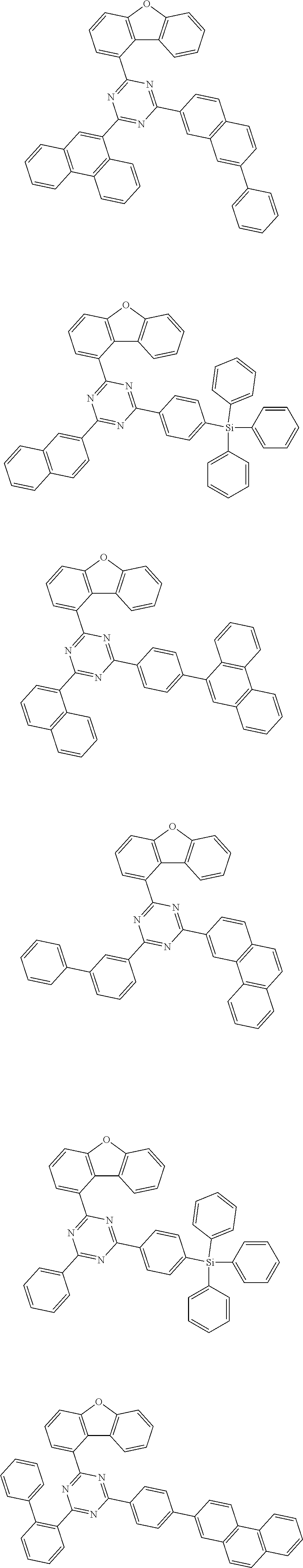

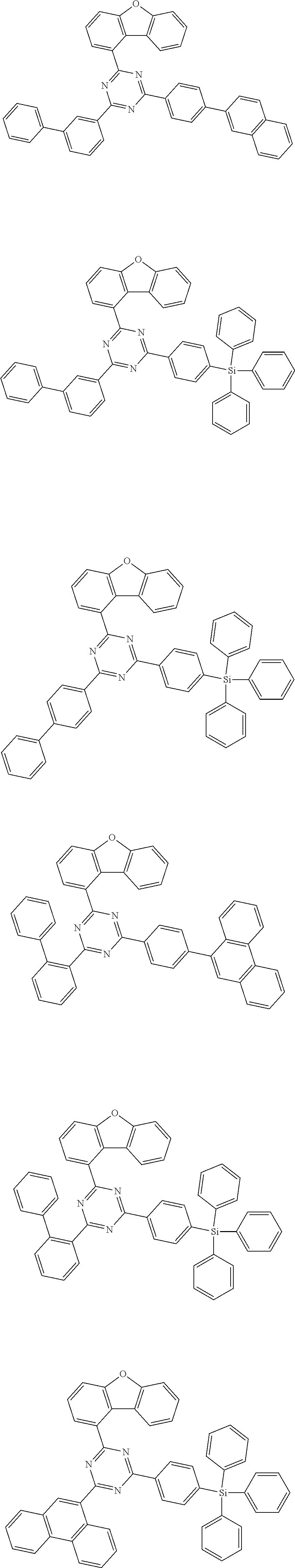

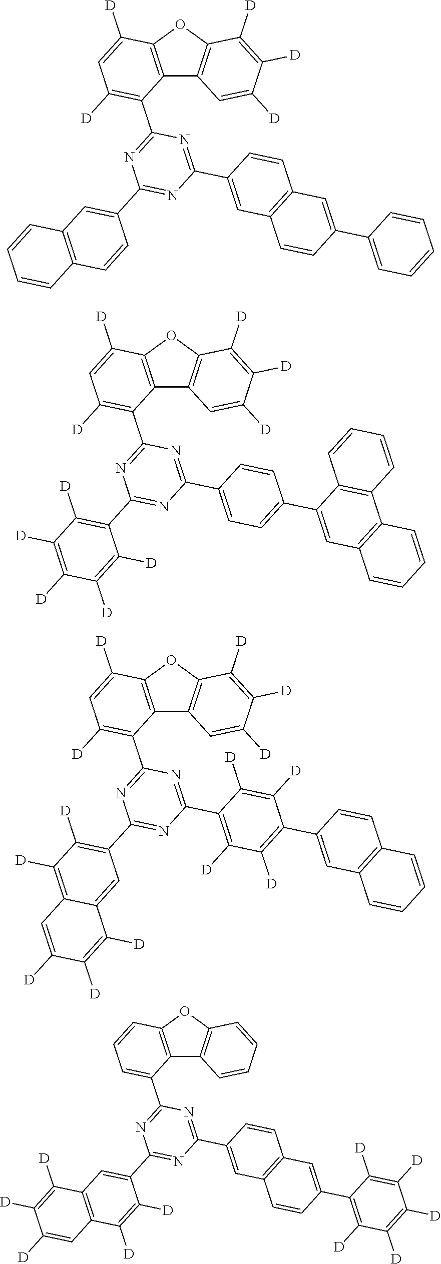

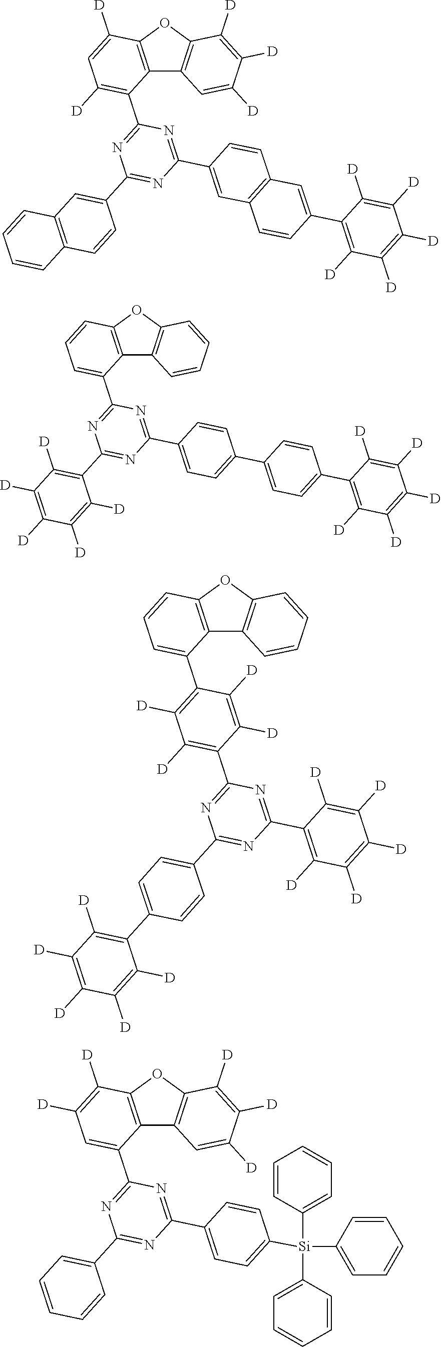

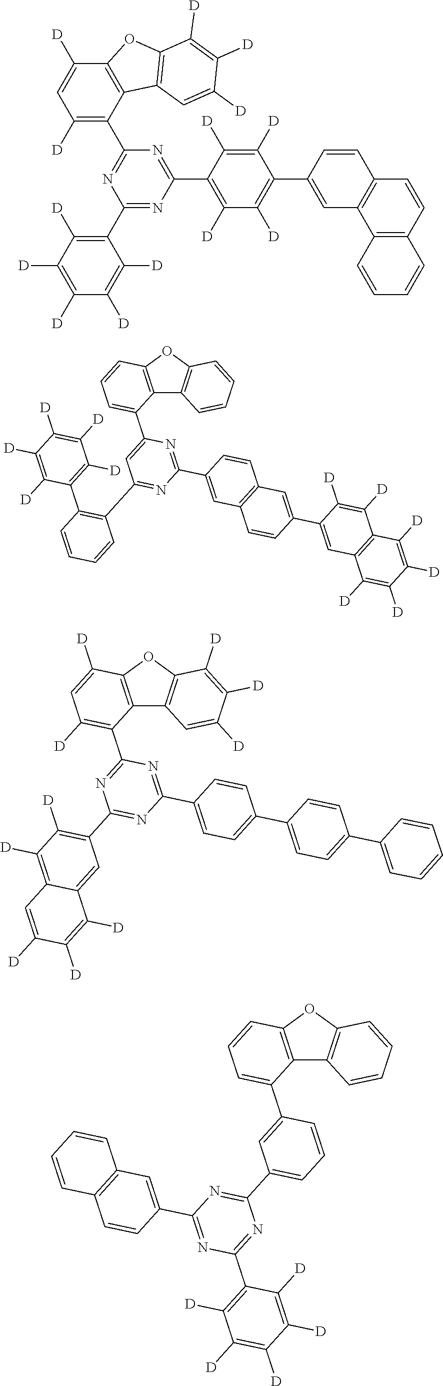

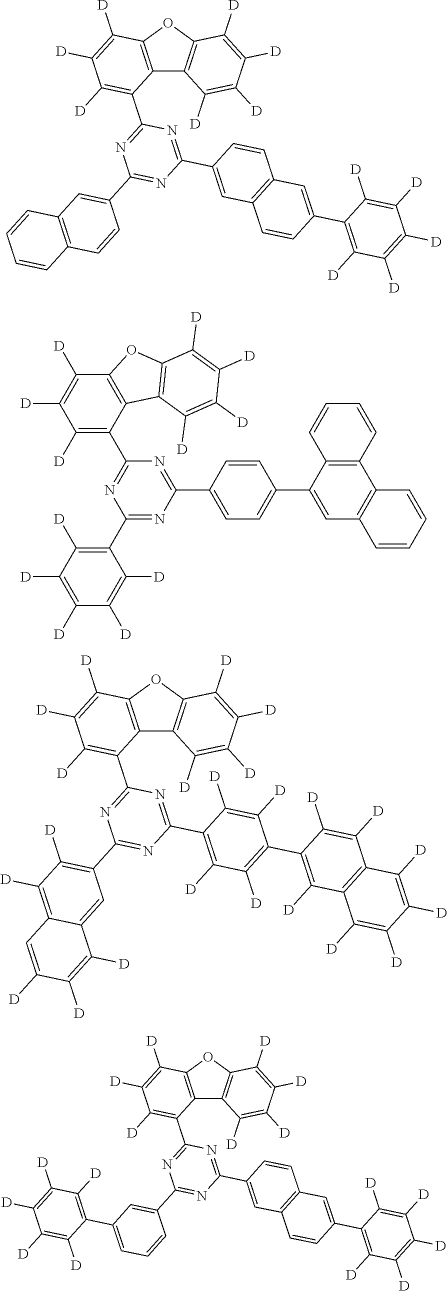

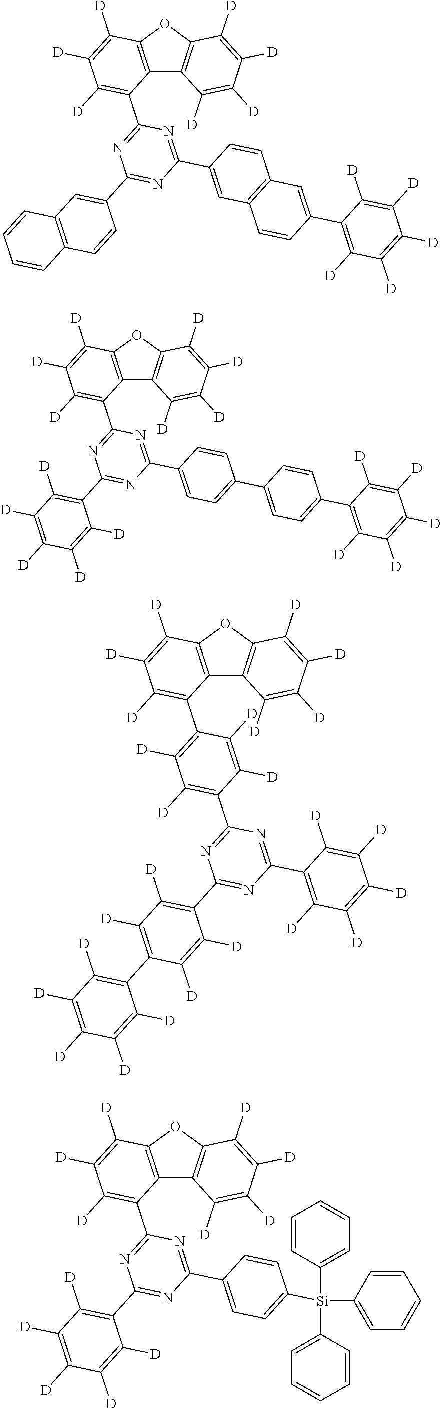

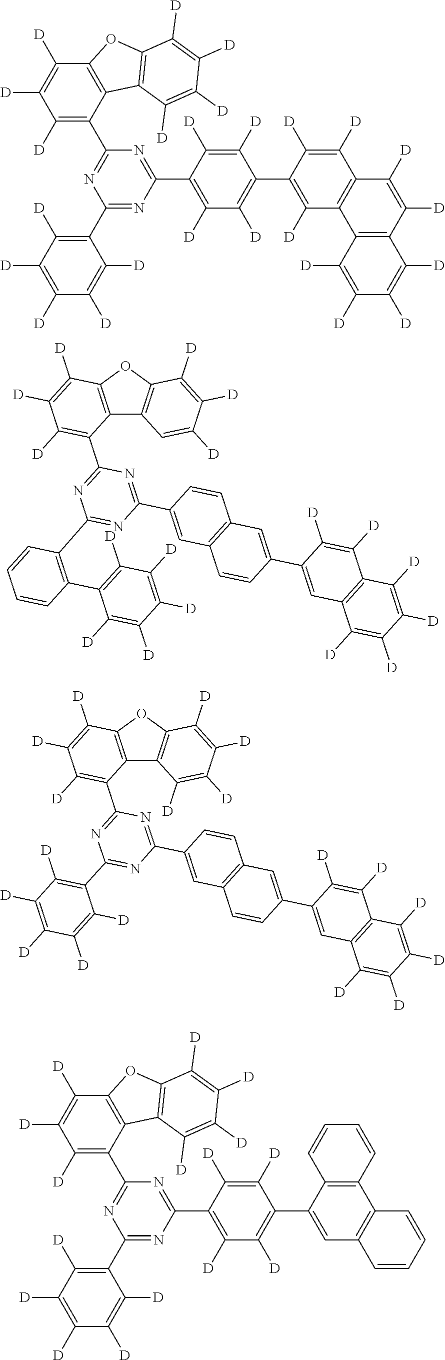

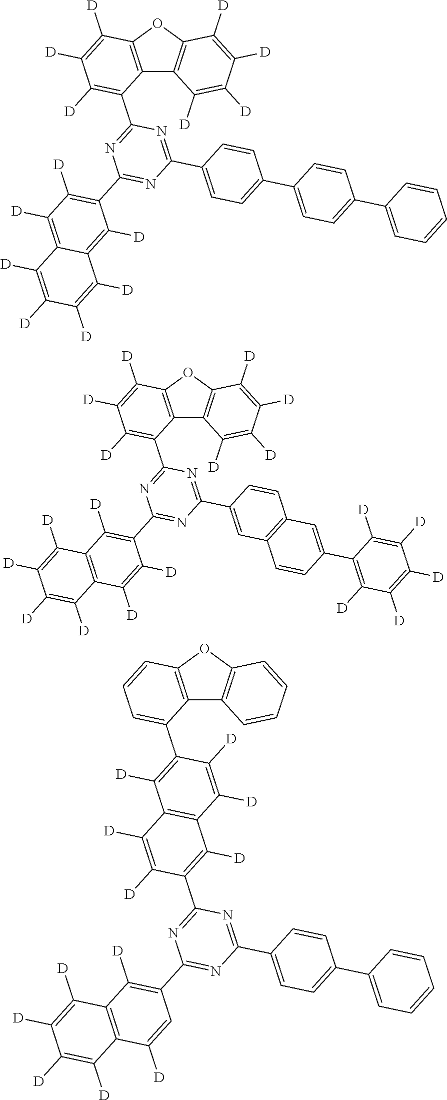

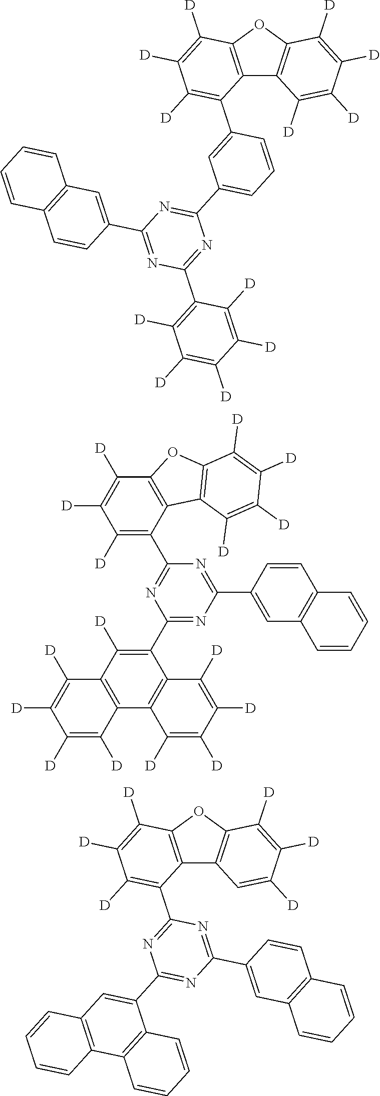

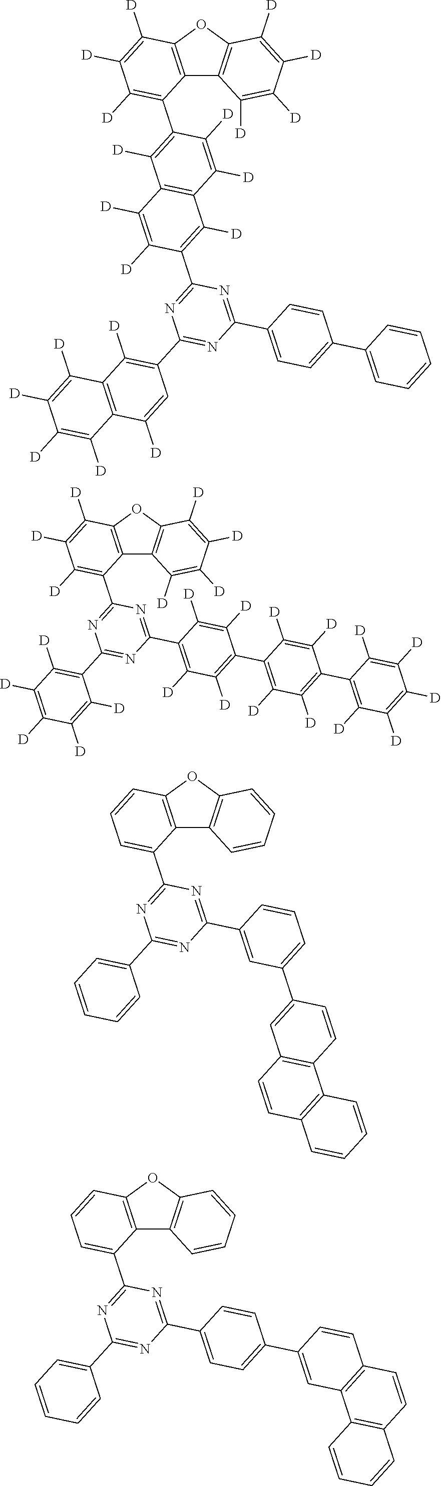

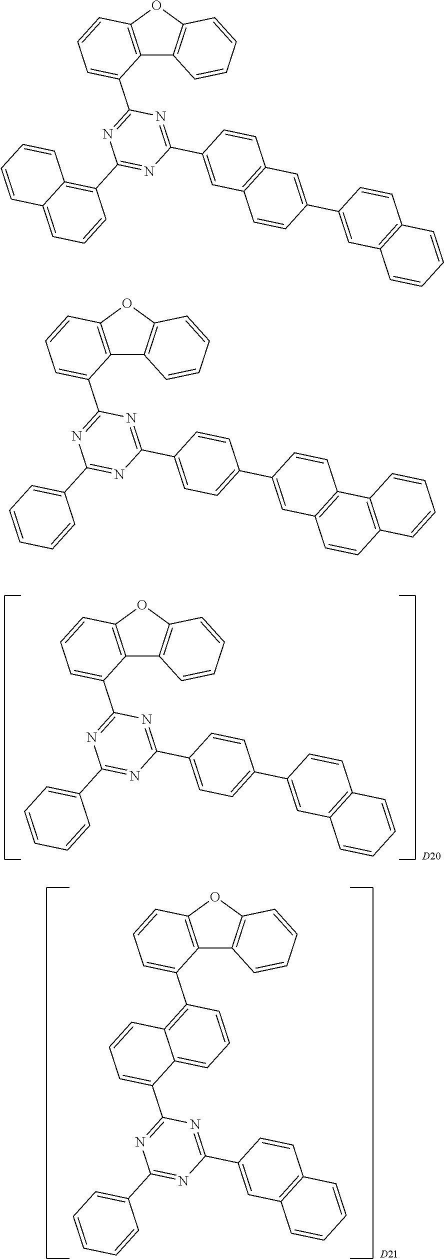

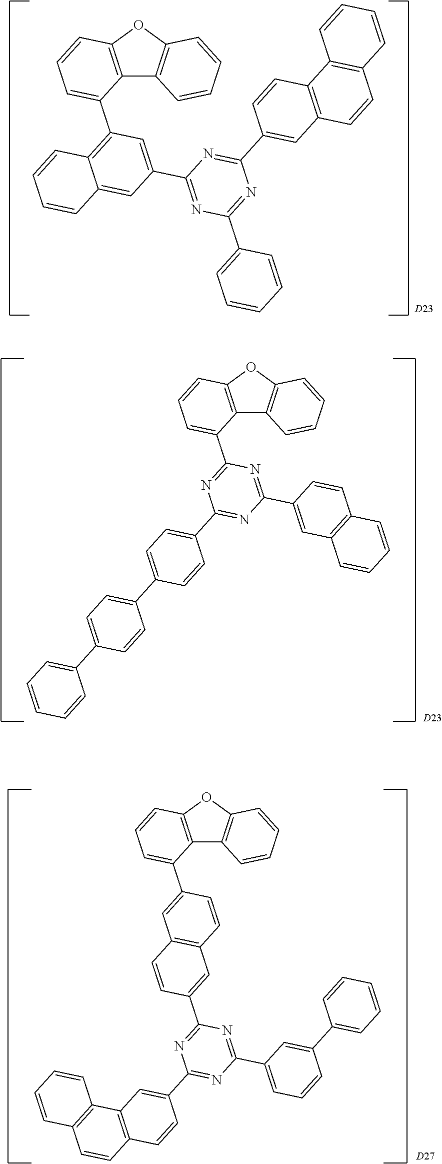

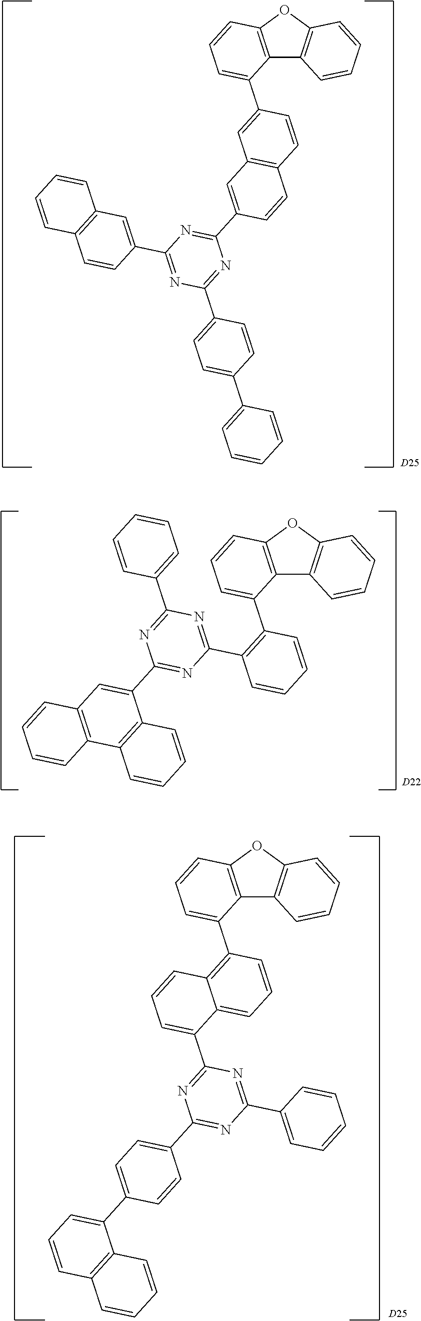

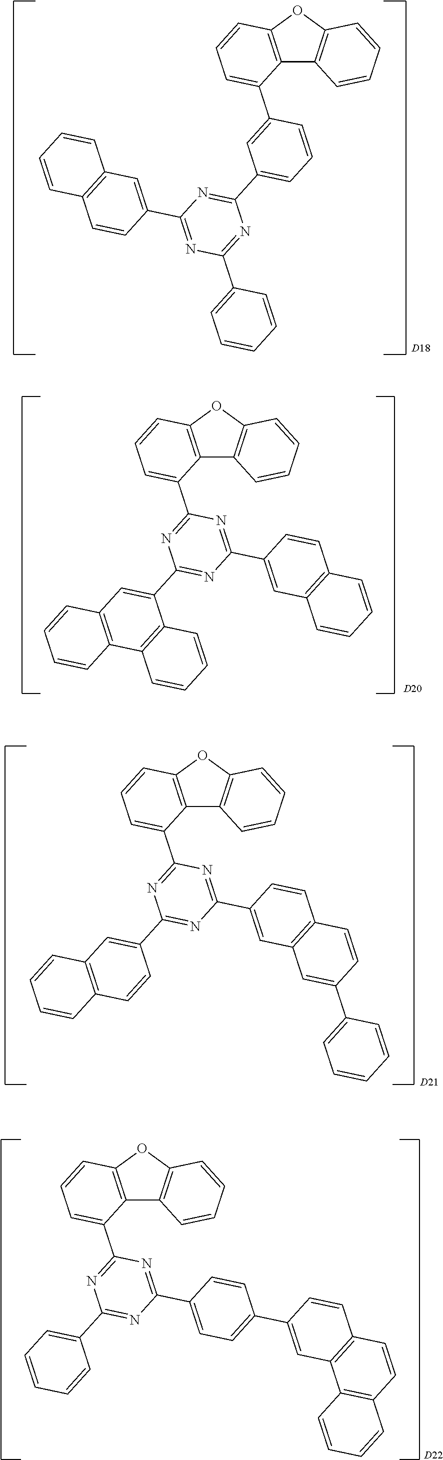

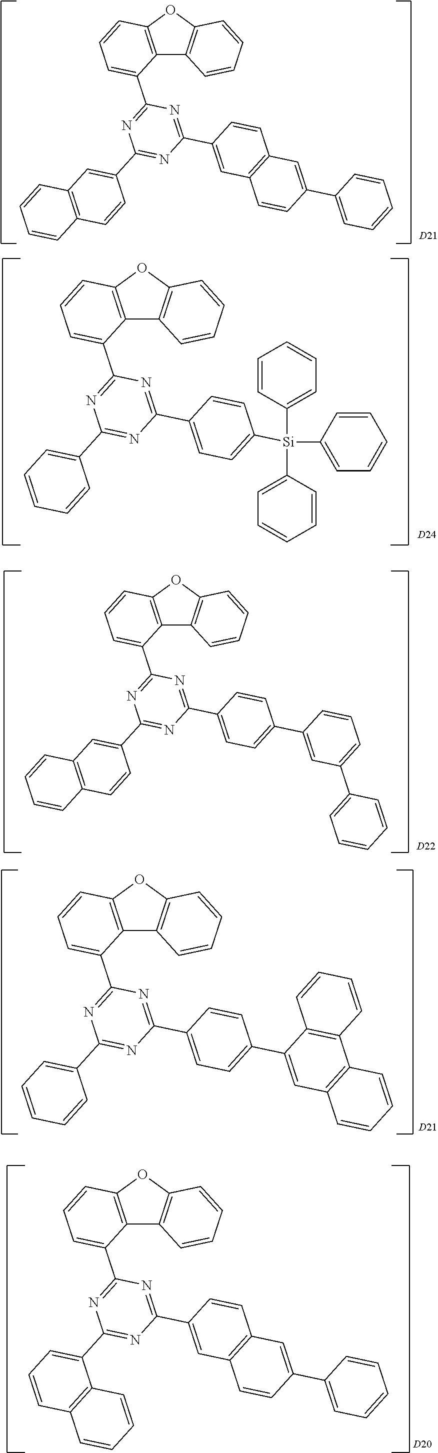

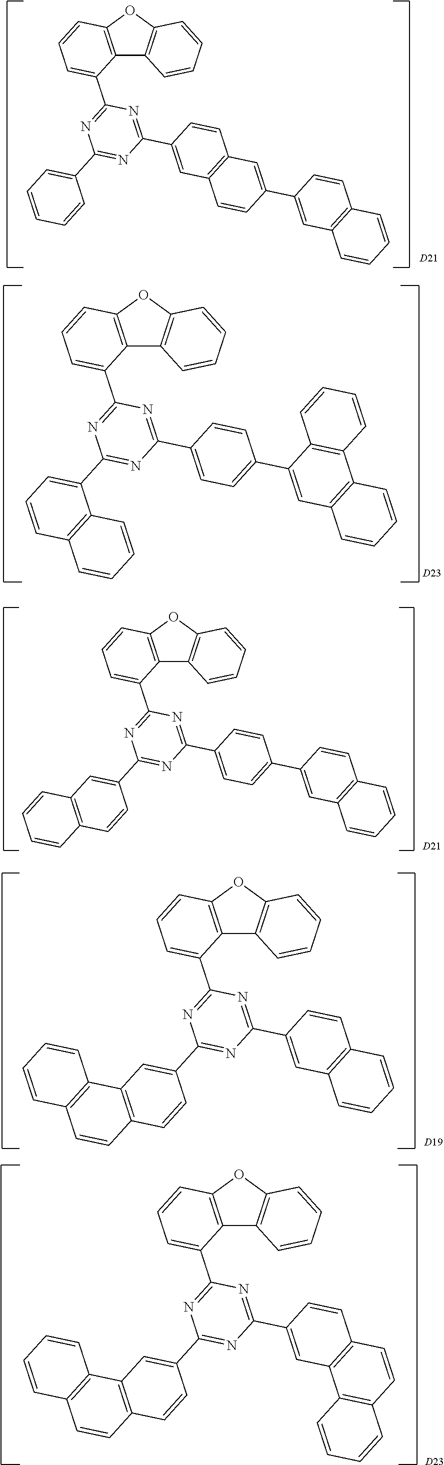

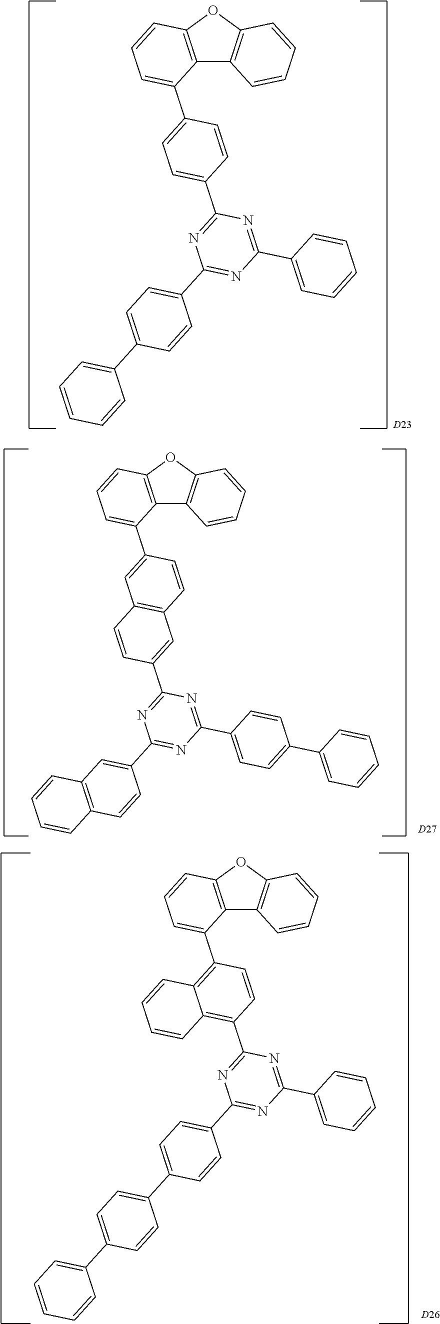

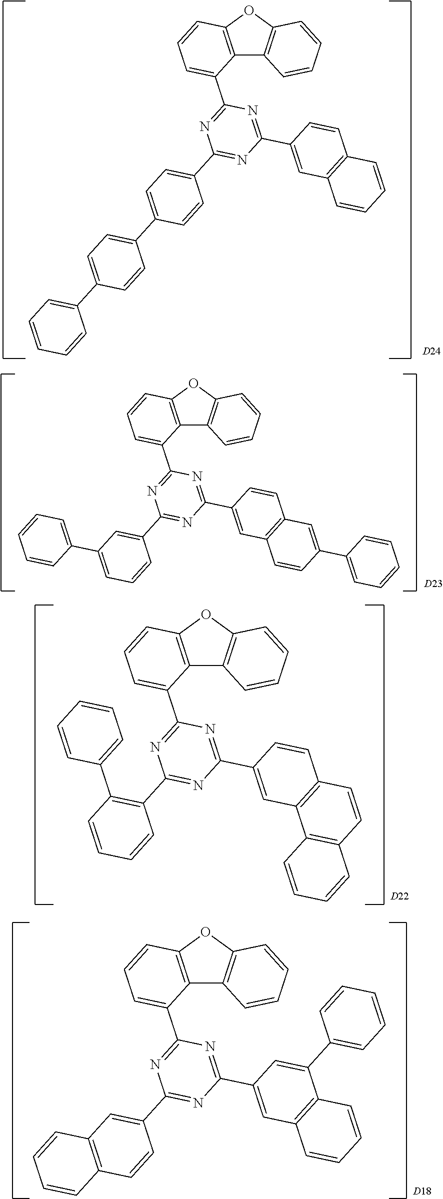

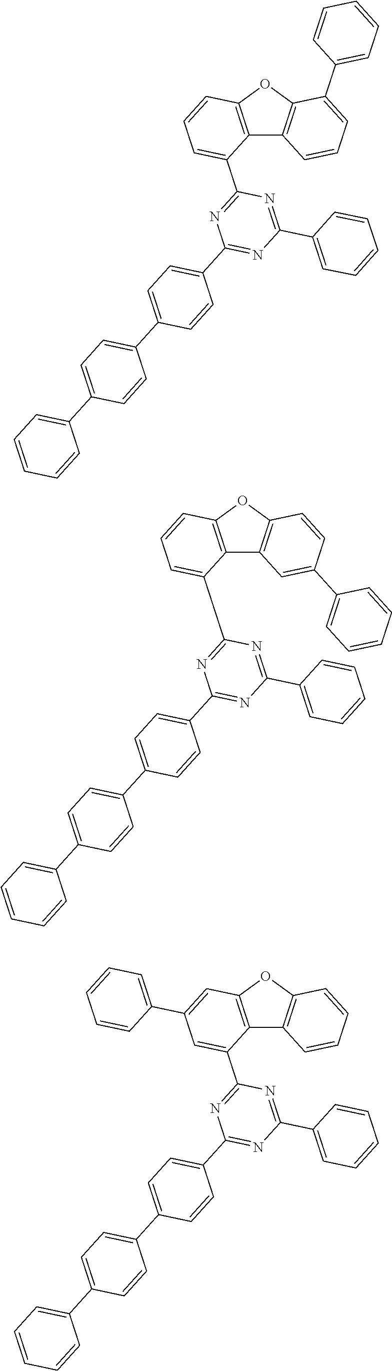 [SMILES: c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1 ; c1ccc(-c2ccc(-c3ccc(-c4nc(-c5ccccc5)nc(-c5cc(-c6ccccc6)cc6oc7ccccc7c56)n4)cc3)cc2)cc1]

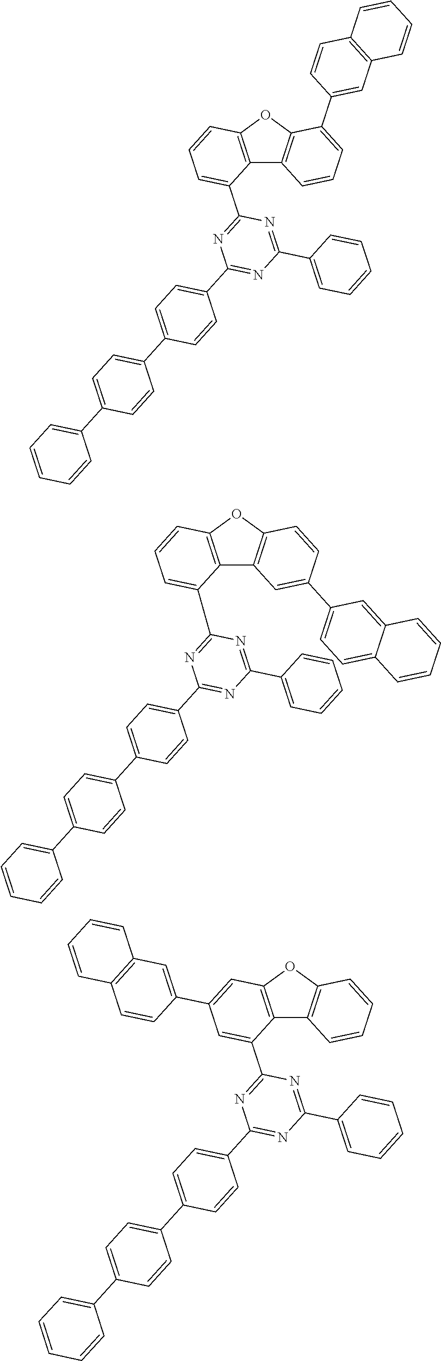 [SMILES: c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1]

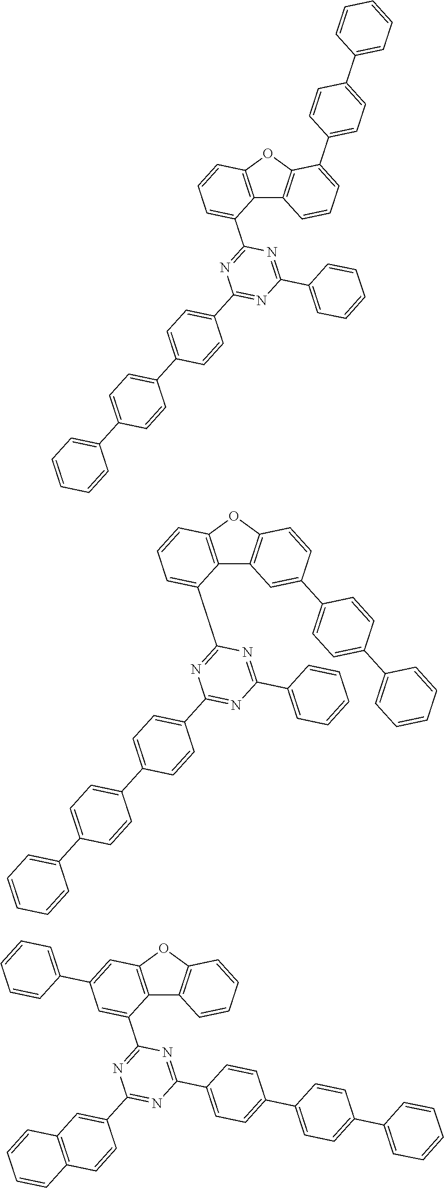

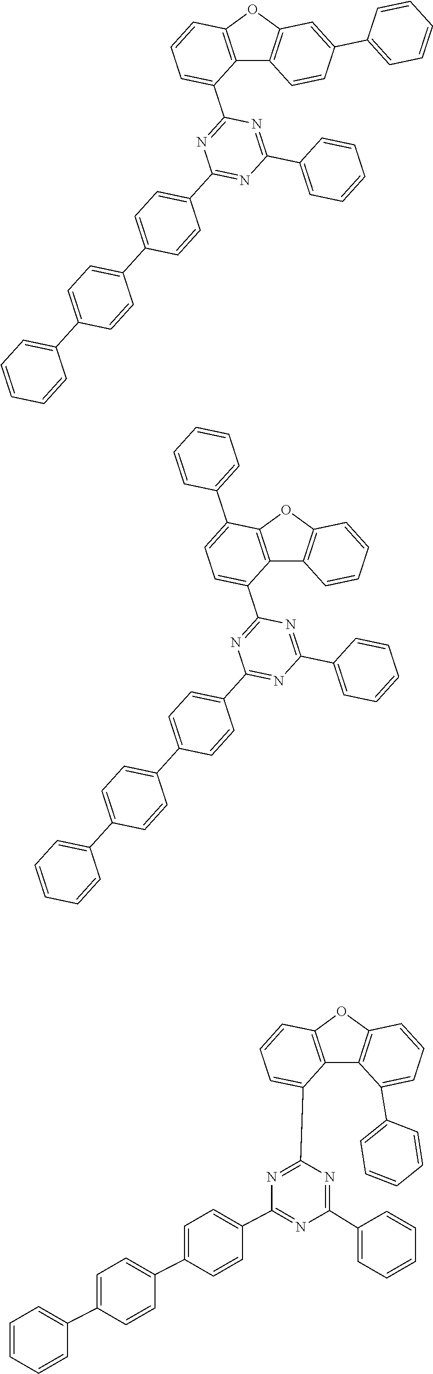 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

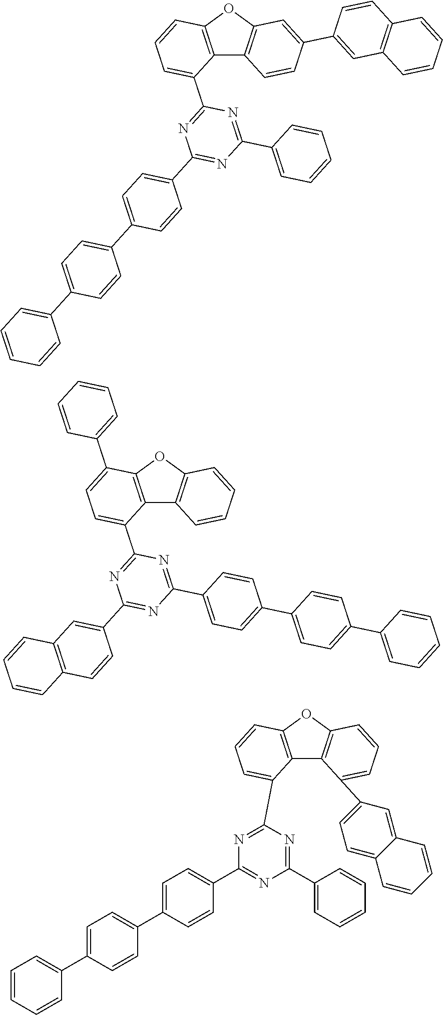

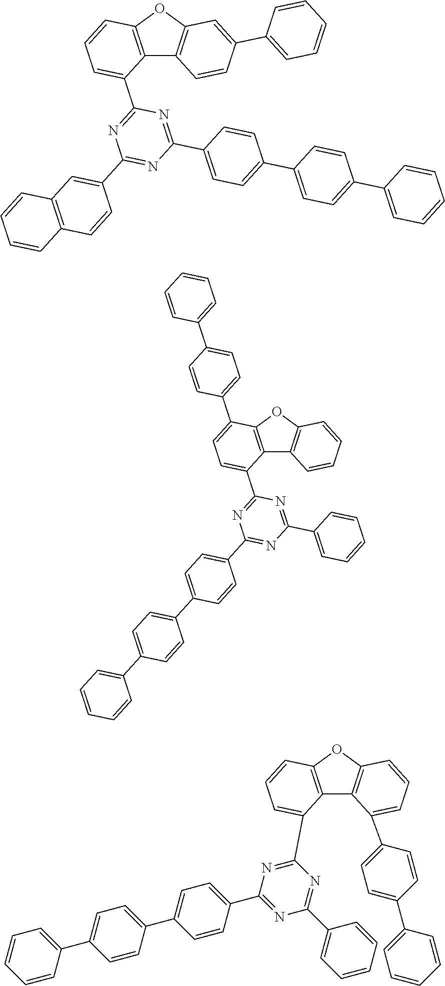 [SMILES: c1ccc(-c2ccc(-c3ccc(-c4nc(-c5ccc6ccccc6c5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

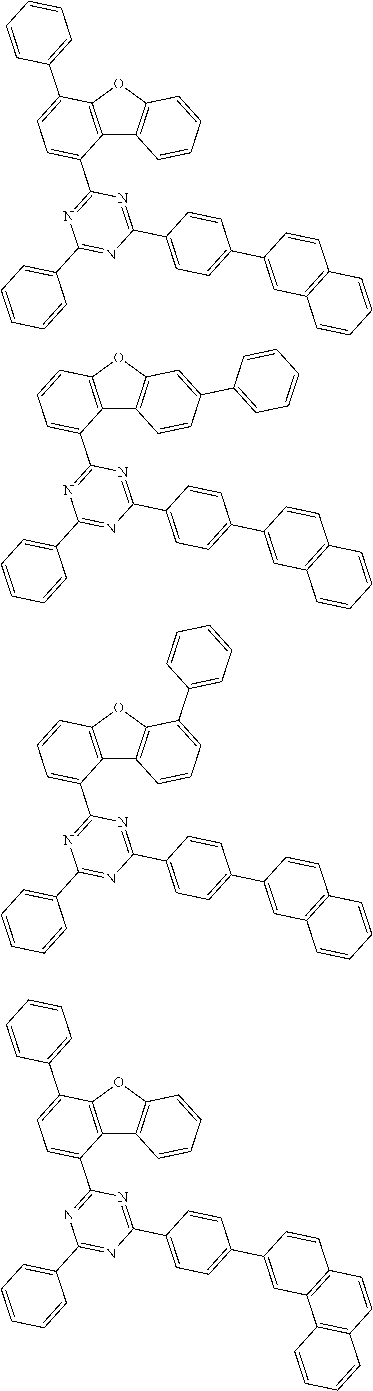

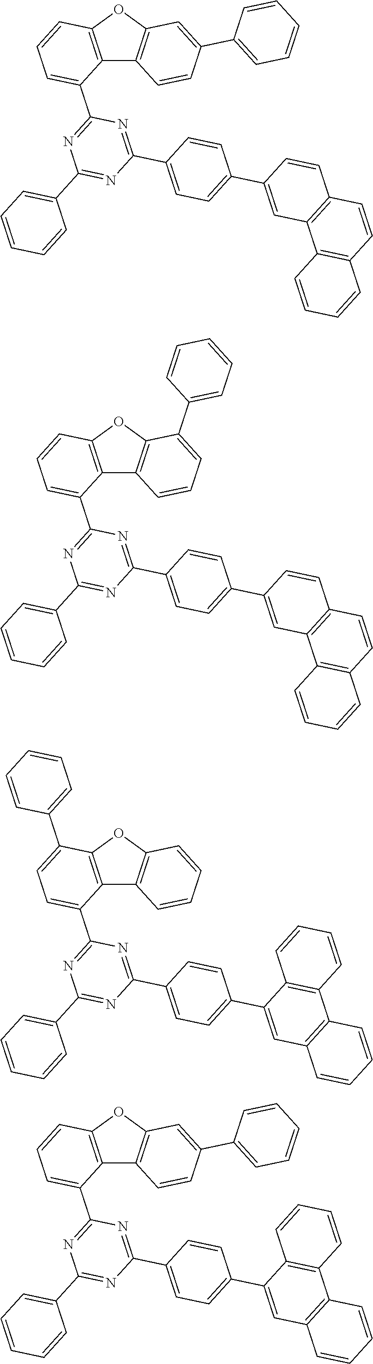 [SMILES: c1ccc(-c2ccc3c(c2)oc2cccc(-c4nc(-c5ccccc5)nc(-c5ccc(-c6ccc7ccc8ccccc8c7c6)cc5)n4)c23)cc1 ; c1ccc(-c2nc(-c3ccc(-c4ccc5ccc6ccccc6c5c4)cc3)nc(-c3cccc4oc5c(-c6ccccc6)cccc5c34)n2)cc1 ; c1ccc(-c2nc(-c3ccc(-c4cc5ccccc5c5ccccc45)cc3)nc(-c3ccc(-c4ccccc4)c4oc5ccccc5c34)n2)cc1 ; c1ccc(-c2ccc3c(c2)oc2cccc(-c4nc(-c5ccccc5)nc(-c5ccc(-c6cc7ccccc7c7ccccc67)cc5)n4)c23)cc1]

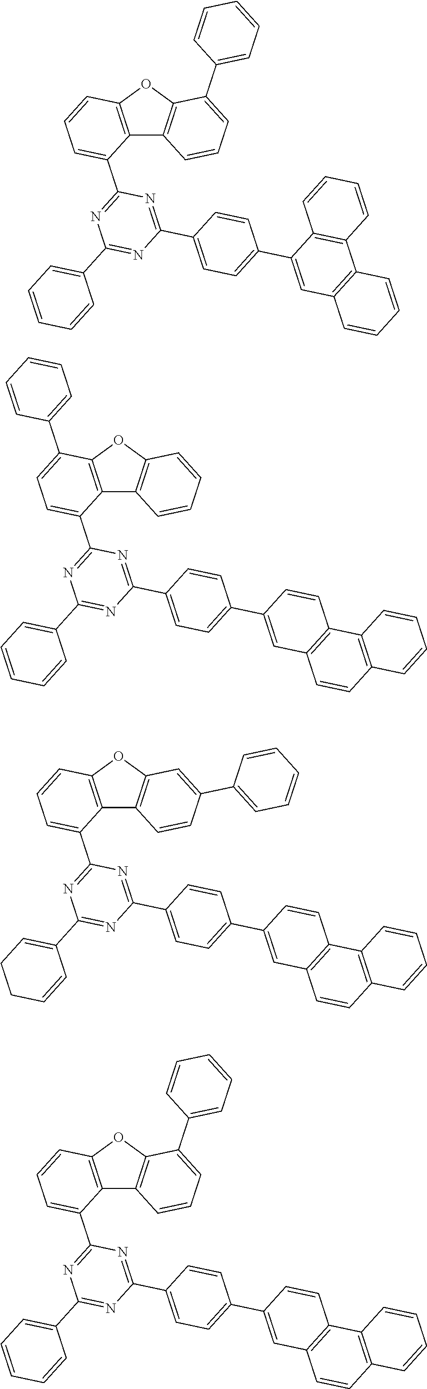

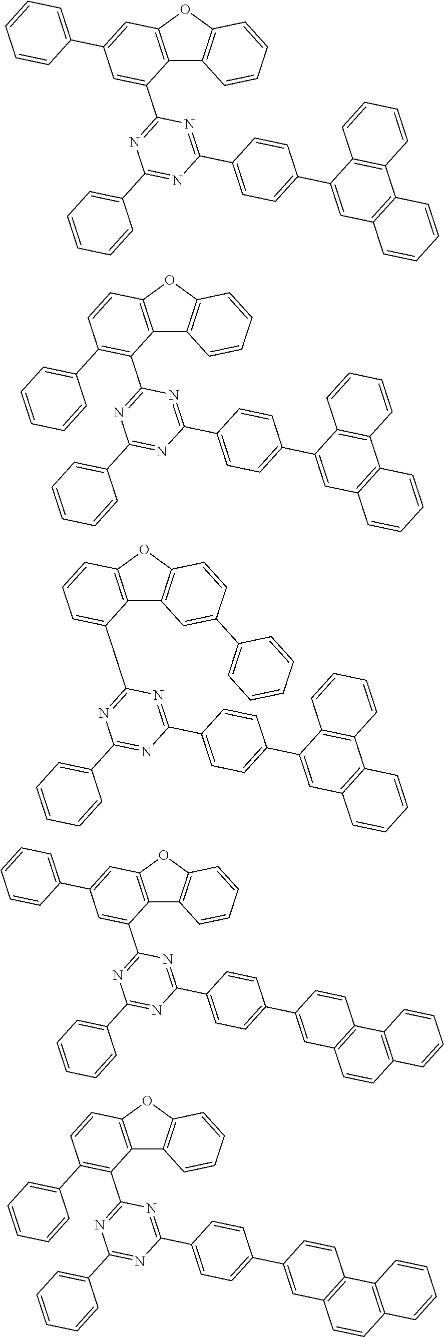

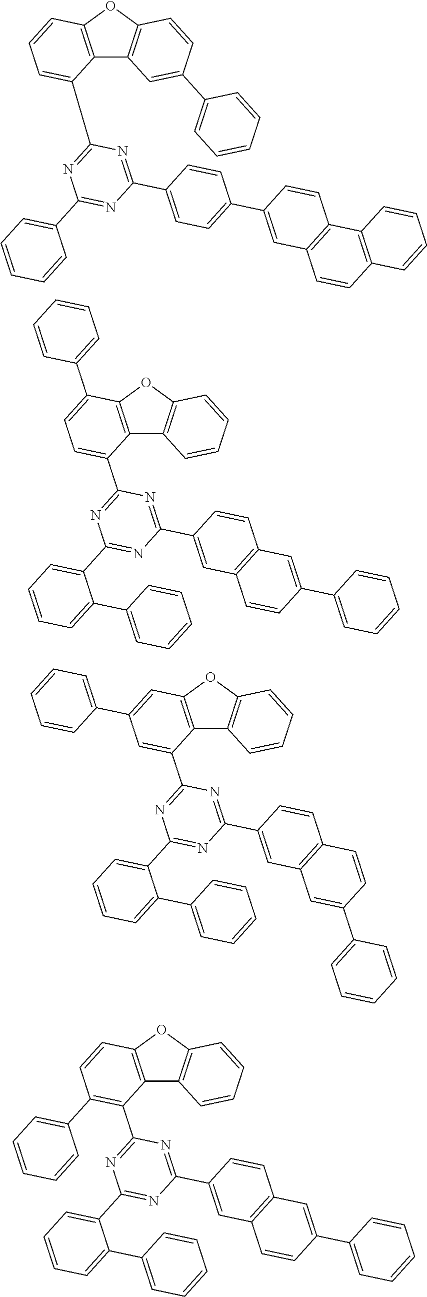

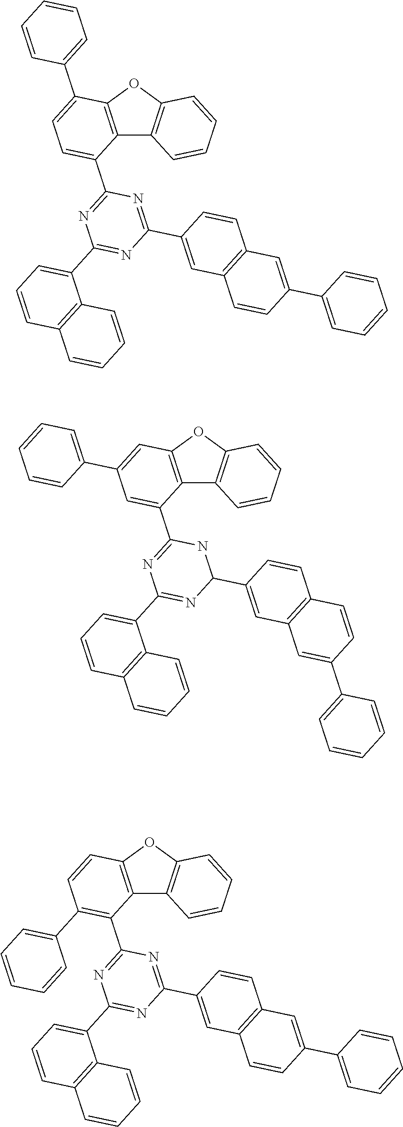

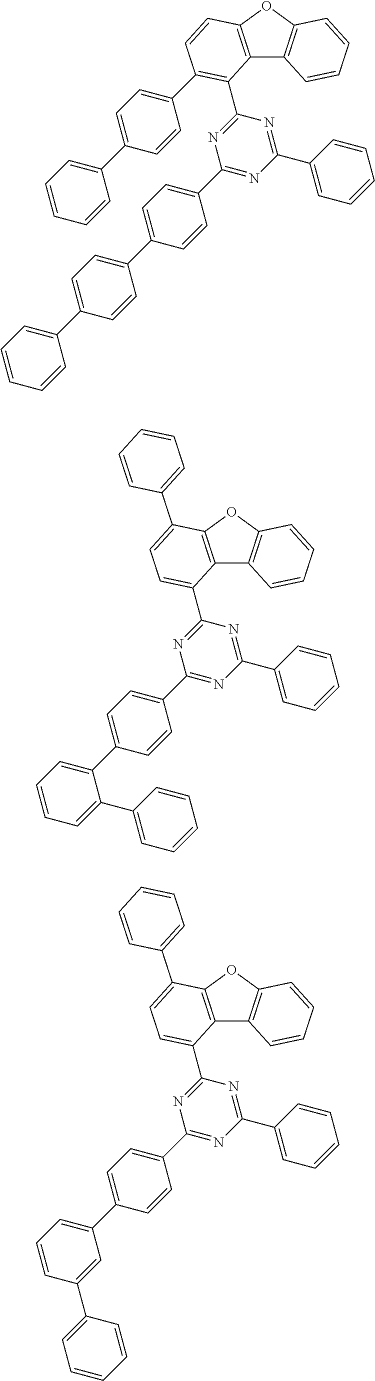

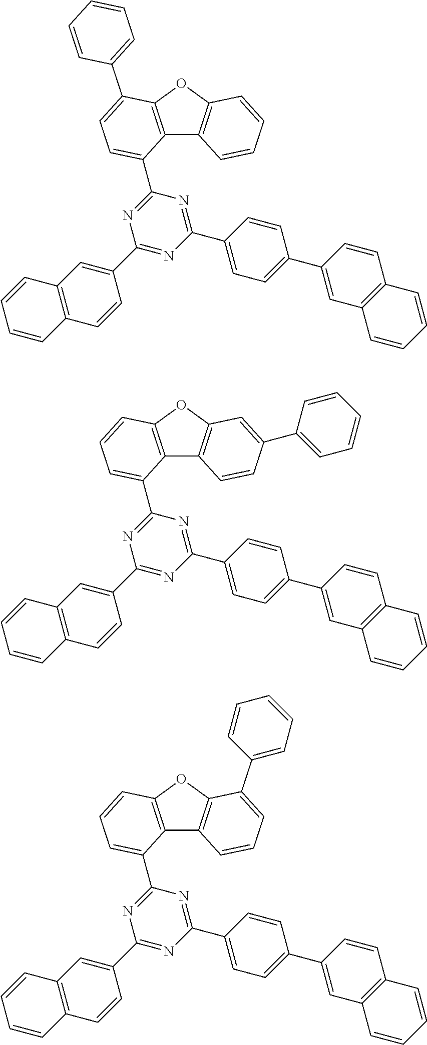 [SMILES: c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1]

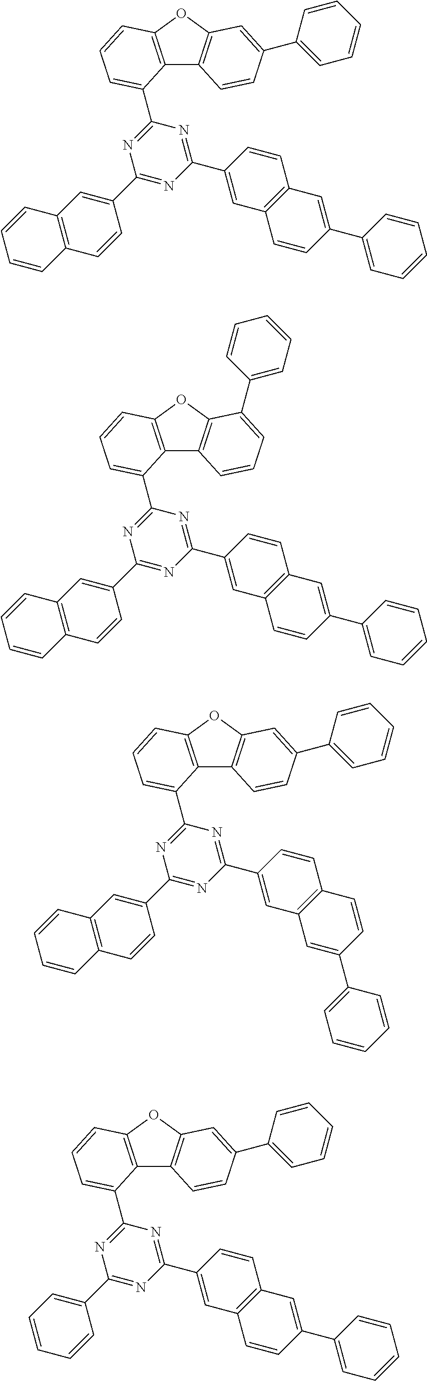 [SMILES: c1ccc(-c2ccc3cc(-c4nc(-c5ccc6ccccc6c5)nc(-c5cccc6oc7cc(-c8ccccc8)ccc7c56)n4)ccc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1 ; c1ccc(-c2ccc3ccc(-c4nc(-c5ccc6ccccc6c5)nc(-c5cccc6oc7cc(-c8ccccc8)ccc7c56)n4)cc3c2)cc1 ; c1ccc(-c2ccc3cc(-c4nc(-c5ccccc5)nc(-c5cccc6oc7cc(-c8ccccc8)ccc7c56)n4)ccc3c2)cc1]

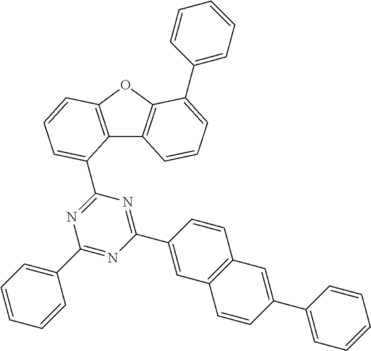 [SMILES: c1ccc(-c2ccc3cc(-c4nc(-c5ccccc5)nc(-c5cccc6oc7c(-c8ccccc8)cccc7c56)n4)ccc3c2)cc1]

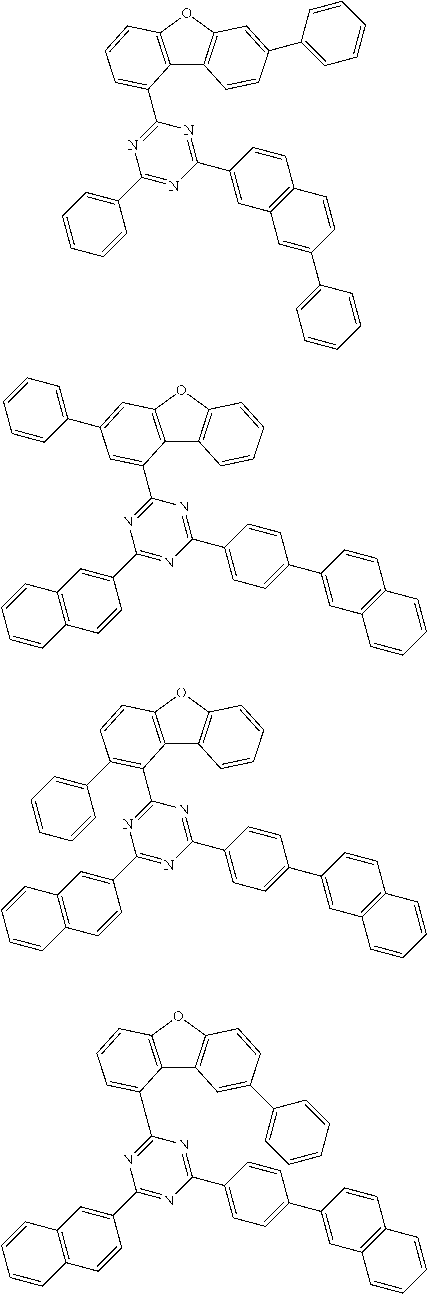 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1 ; c1ccc(-c2ccc3oc4ccccc4c3c2-c2nc(-c3ccc(-c4ccc5ccccc5c4)cc3)nc(-c3ccc4ccccc4c3)n2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

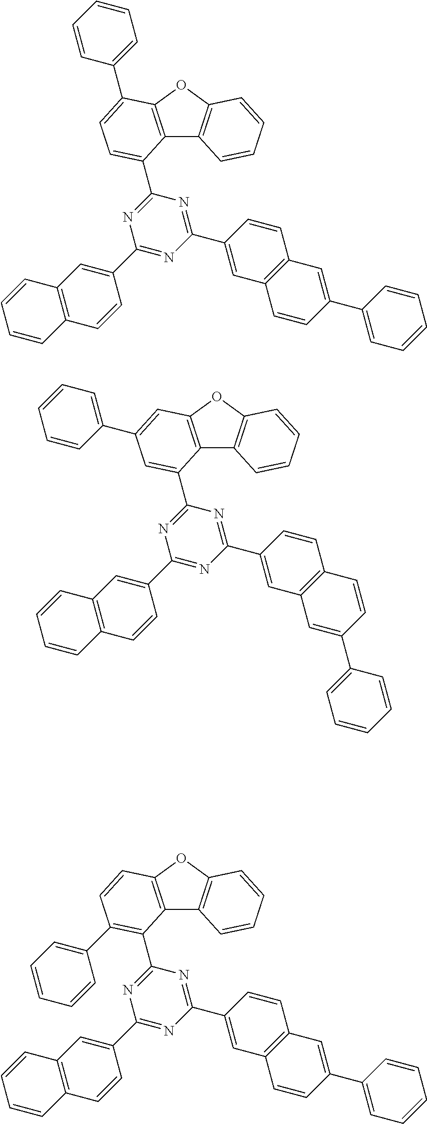

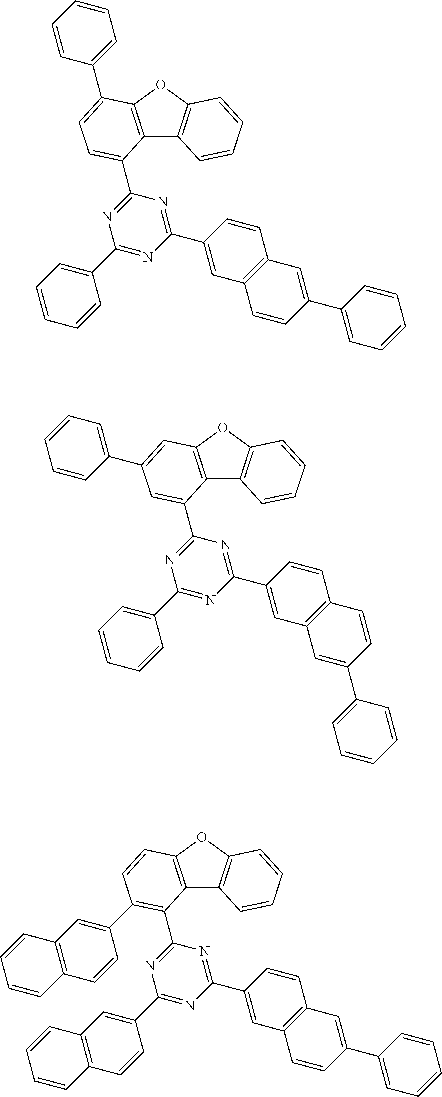 [SMILES: c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3ccc(-c4nc(-c5ccccc5)nc(-c5cc(-c6ccccc6)cc6oc7ccccc7c56)n4)cc3c2)cc1 ; c1ccc(-c2ccc3cc(-c4nc(-c5ccc6ccccc6c5)nc(-c5c(-c6ccc7ccccc7c6)ccc6oc7ccccc7c56)n4)ccc3c2)cc1]

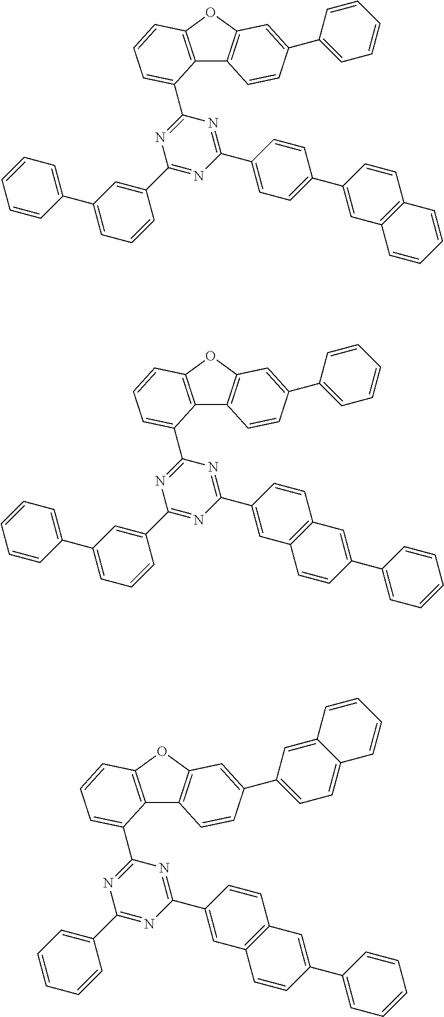 [SMILES: c1ccc(-c2cccc(-c3nc(-c4ccc(-c5ccc6ccccc6c5)cc4)nc(-c4cccc5oc6cc(-c7ccccc7)ccc6c45)n3)c2)cc1 ; c1ccc(-c2cccc(-c3nc(-c4ccc5cc(-c6ccccc6)ccc5c4)nc(-c4cccc5oc6cc(-c7ccccc7)ccc6c45)n3)c2)cc1 ; c1ccc(-c2ccc3cc(-c4nc(-c5ccccc5)nc(-c5cccc6oc7cc(-c8ccc9ccccc9c8)ccc7c56)n4)ccc3c2)cc1]

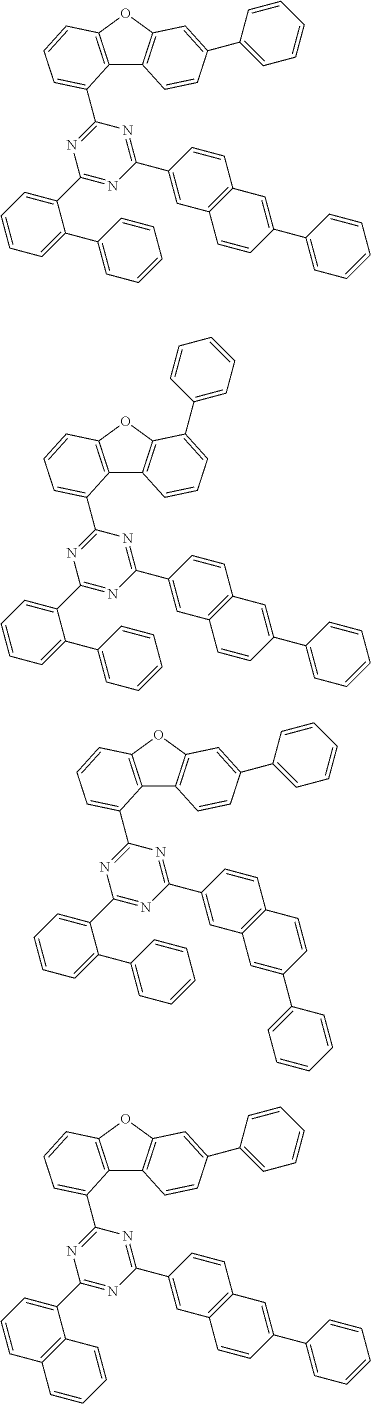

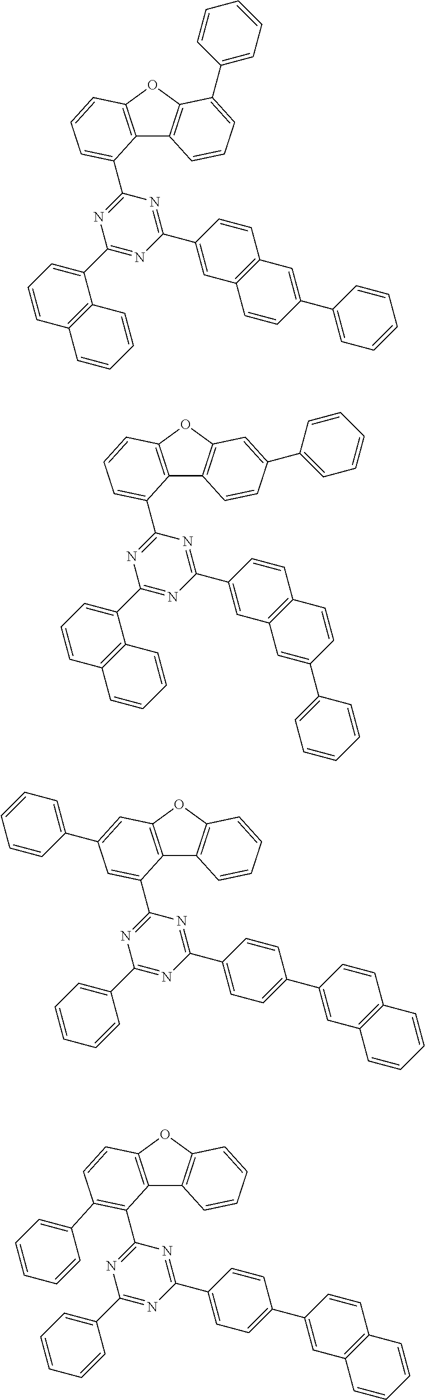 [SMILES: c1ccc(-c2ccc3cc(-c4nc(-c5cccc6ccccc56)nc(-c5cccc6oc7c(-c8ccccc8)cccc7c56)n4)ccc3c2)cc1 ; c1ccc(-c2ccc3ccc(-c4nc(-c5cccc6ccccc56)nc(-c5cccc6oc7cc(-c8ccccc8)ccc7c56)n4)cc3c2)cc1 ; c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2nc(-c3ccc(-c4ccc5ccccc5c4)cc3)nc(-c3c(-c4ccccc4)ccc4oc5ccccc5c34)n2)cc1]

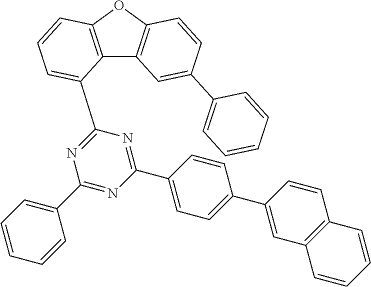 [SMILES: c1ccc(-c2ccc3oc4cccc(-c5nc(-c6ccccc6)nc(-c6ccc(-c7ccc8ccccc8c7)cc6)n5)c4c3c2)cc1]

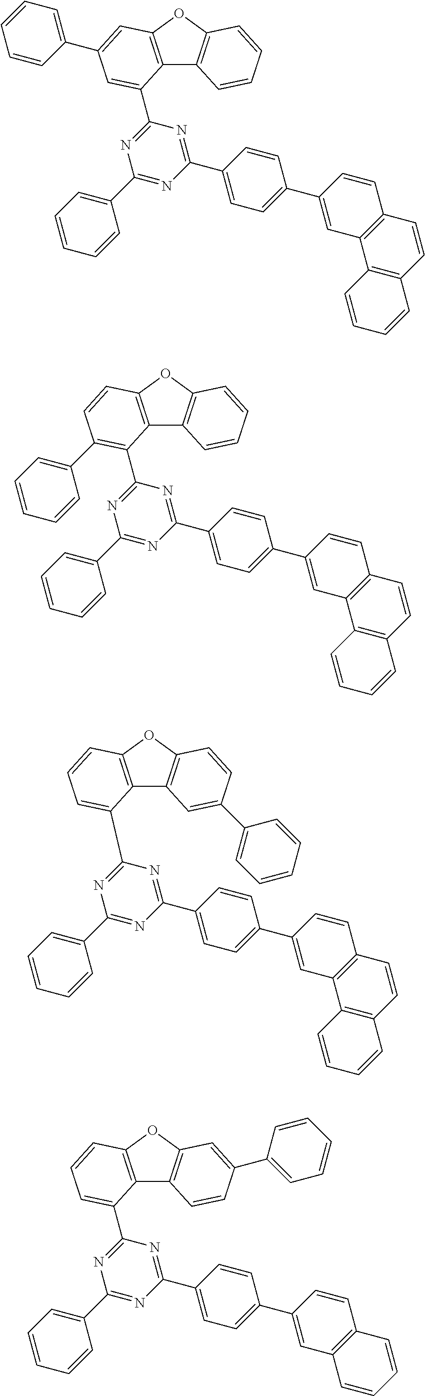 [SMILES: c1ccc(-c2cc(-c3nc(-c4ccccc4)nc(-c4ccc(-c5ccc6ccc7ccccc7c6c5)cc4)n3)c3c(c2)oc2ccccc23)cc1 ; c1ccc(-c2nc(-c3ccc(-c4ccc5ccc6ccccc6c5c4)cc3)nc(-c3c(-c4ccccc4)ccc4oc5ccccc5c34)n2)cc1 ; c1ccc(-c2ccc3oc4cccc(-c5nc(-c6ccccc6)nc(-c6ccc(-c7ccc8ccc9ccccc9c8c7)cc6)n5)c4c3c2)cc1 ; c1ccc(-c2ccc3c(c2)oc2cccc(-c4nc(-c5ccccc5)nc(-c5ccc(-c6ccc7ccccc7c6)cc5)n4)c23)cc1]

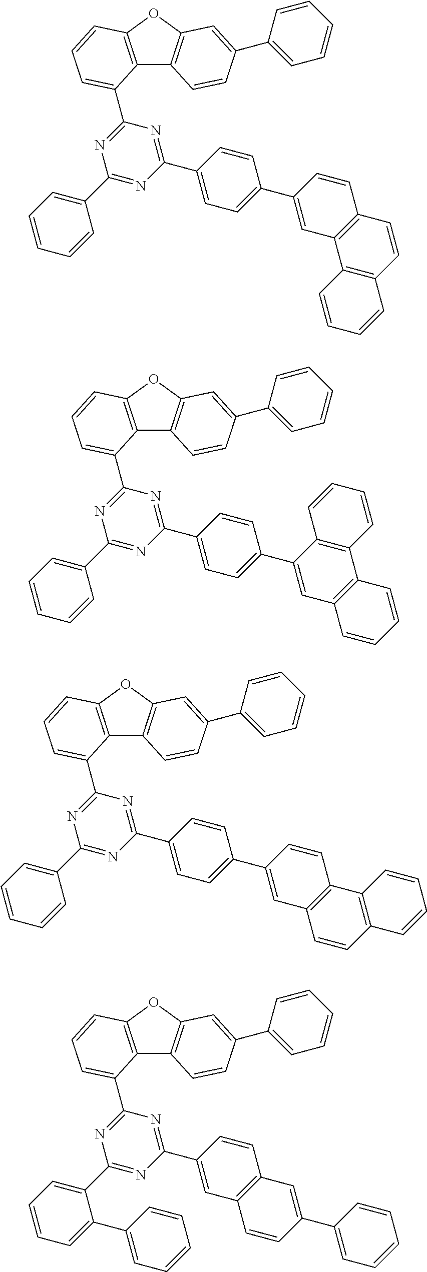

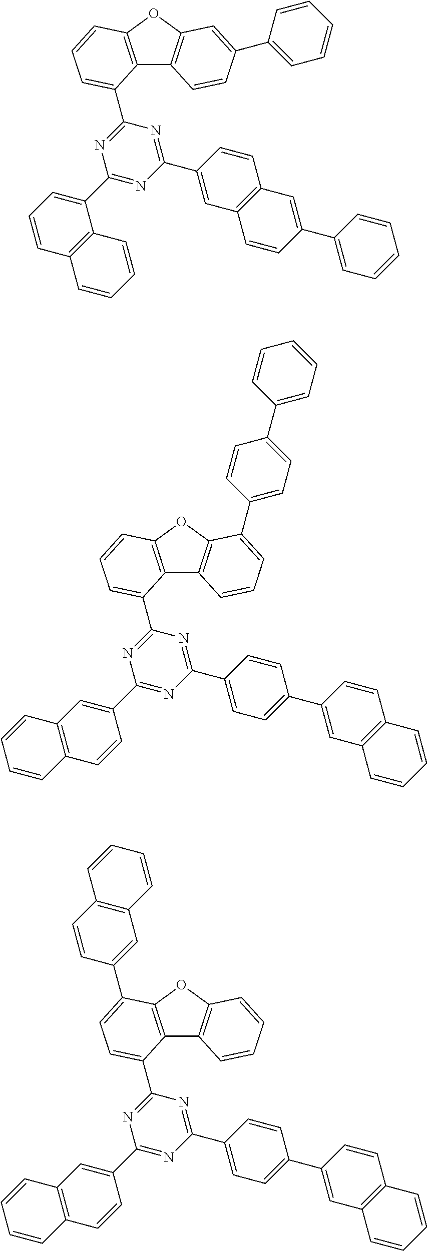 [SMILES: c1ccc(-c2ccc3cc(-c4nc(-c5cccc6ccccc56)nc(-c5cccc6oc7cc(-c8ccccc8)ccc7c56)n4)ccc3c2)cc1 ; c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1]

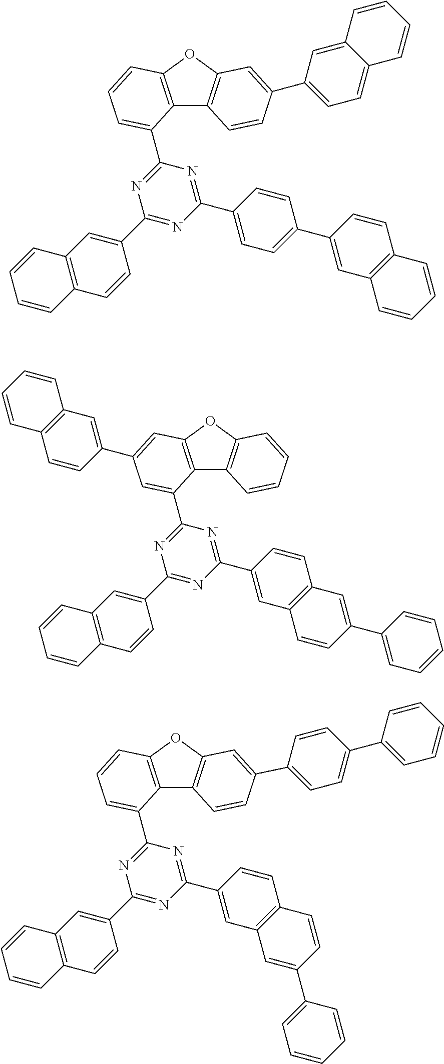

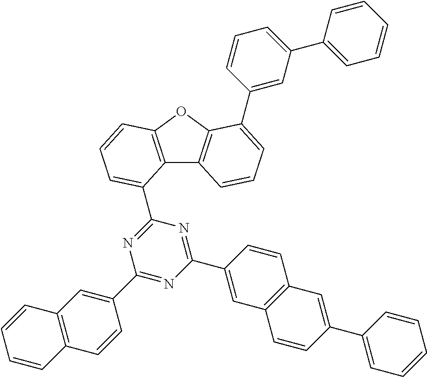 [SMILES: c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1]

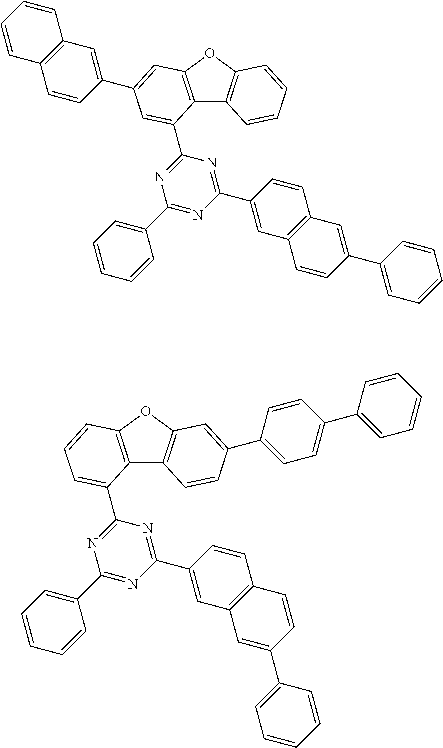 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1]

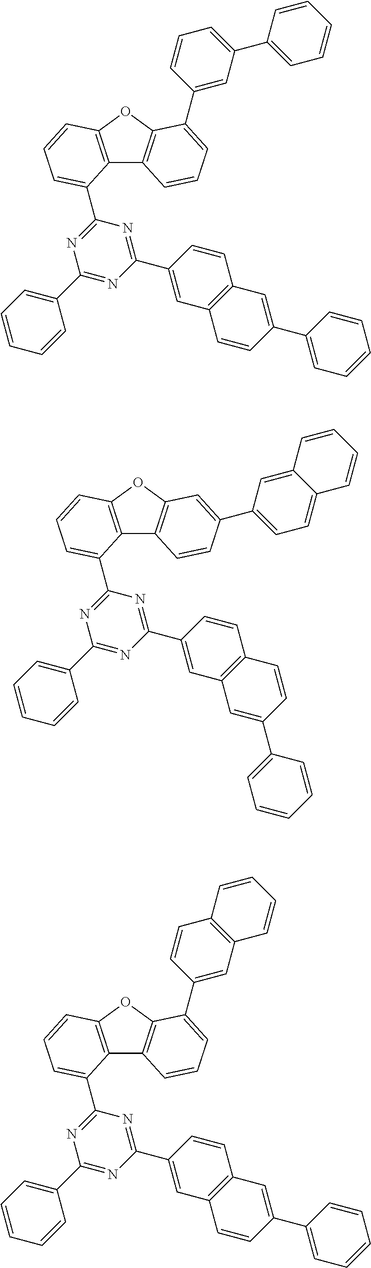 [SMILES: c1ccc(-c2cccc(-c3cccc4c3oc3cccc(-c5nc(-c6ccccc6)nc(-c6ccc7cc(-c8ccccc8)ccc7c6)n5)c34)c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

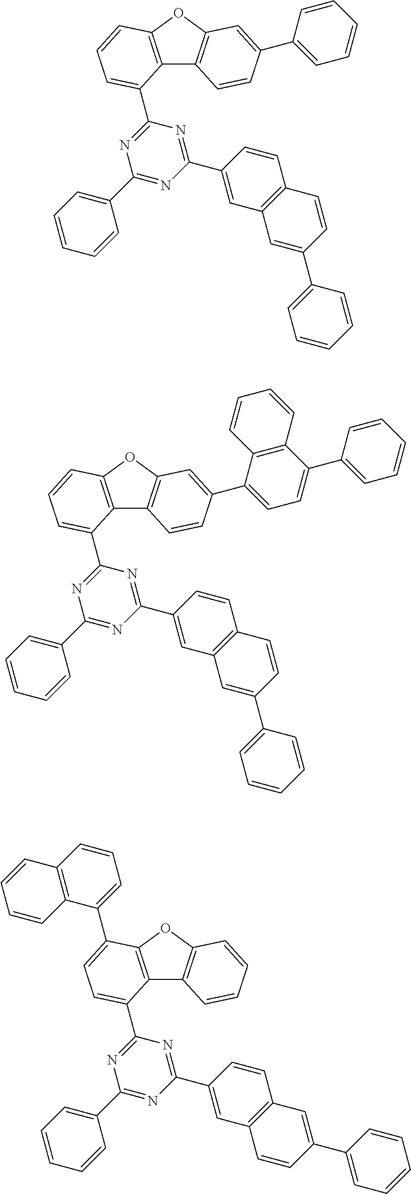 [SMILES: c1ccc(-c2ccc3ccc(-c4nc(-c5ccccc5)nc(-c5cccc6oc7cc(-c8ccccc8)ccc7c56)n4)cc3c2)cc1 ; c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1]

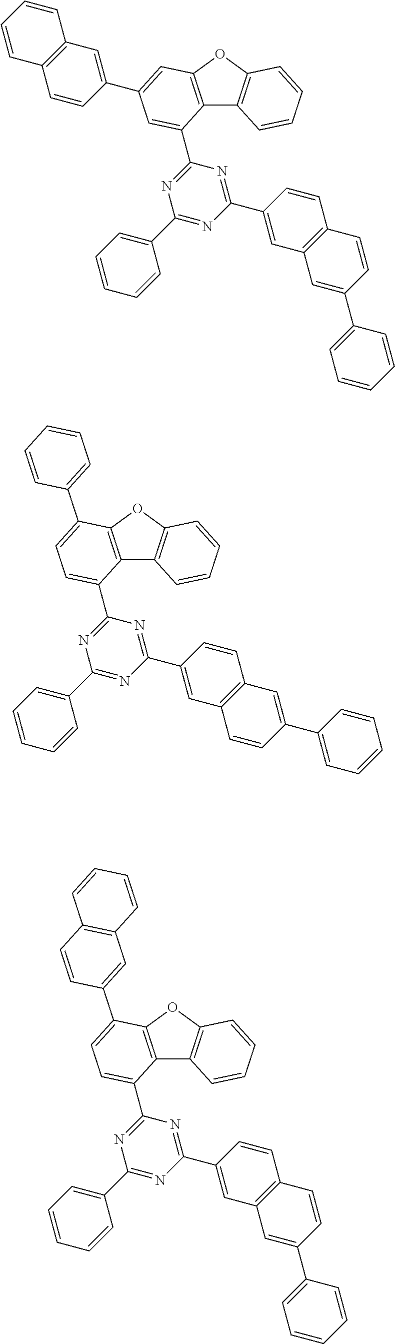 [SMILES: c1ccc(-c2ccc3ccc(-c4nc(-c5ccccc5)nc(-c5cc(-c6ccc7ccccc7c6)cc6oc7ccccc7c56)n4)cc3c2)cc1 ; c1ccc(-c2ccc3cc(-c4nc(-c5ccccc5)nc(-c5ccc(-c6ccccc6)c6oc7ccccc7c56)n4)ccc3c2)cc1 ; c1ccc(-c2ccc3ccc(-c4nc(-c5ccccc5)nc(-c5ccc(-c6ccc7ccccc7c6)c6oc7ccccc7c56)n4)cc3c2)cc1]

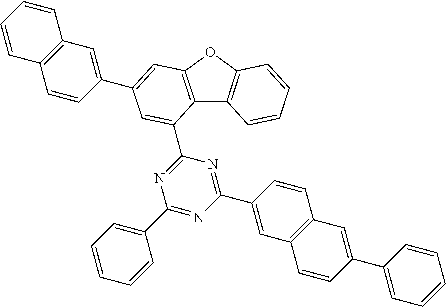 [SMILES: c1ccc(-c2ccc3cc(-c4nc(-c5ccccc5)nc(-c5cc(-c6ccc7ccccc7c6)cc6oc7ccccc7c56)n4)ccc3c2)cc1]

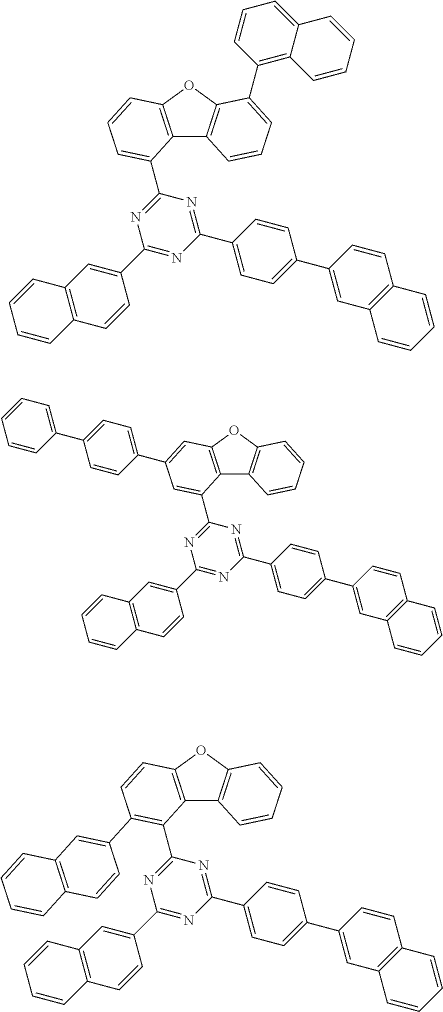 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1]

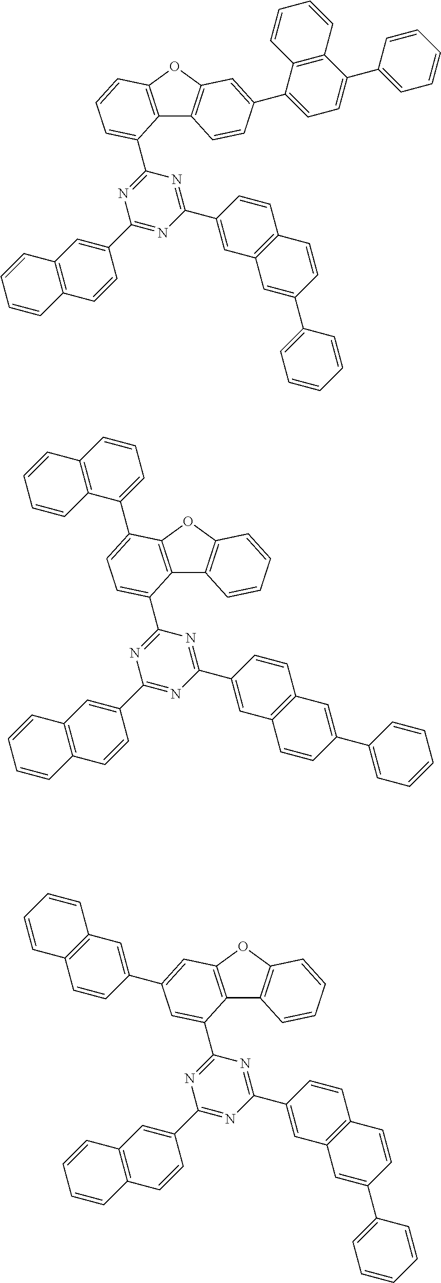 [SMILES: c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1]

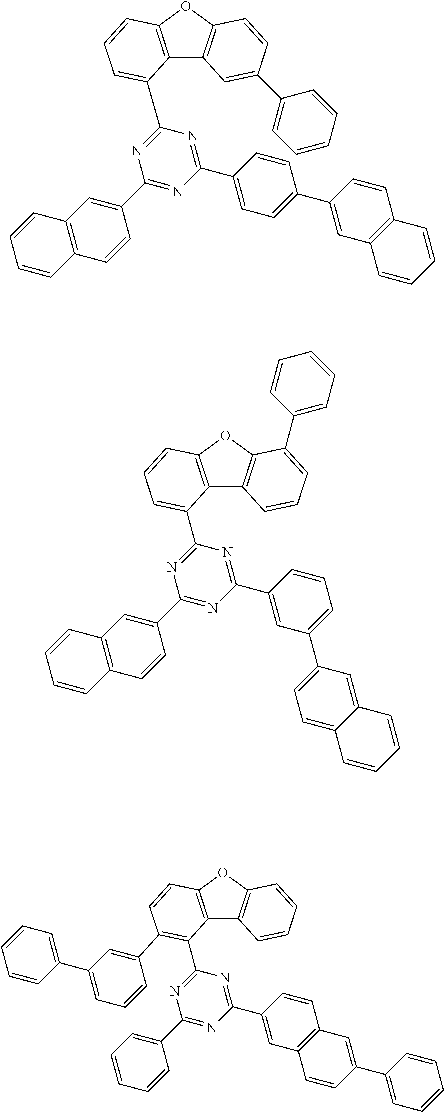 [SMILES: c1ccc(-c2ccc3oc4cccc(-c5nc(-c6ccc(-c7ccc8ccccc8c7)cc6)nc(-c6ccc7ccccc7c6)n5)c4c3c2)cc1 ; c1ccc(-c2cccc3c2oc2cccc(-c4nc(-c5cccc(-c6ccc7ccccc7c6)c5)nc(-c5ccc6ccccc6c5)n4)c23)cc1 ; c1ccc(-c2cccc(-c3ccc4oc5ccccc5c4c3-c3nc(-c4ccccc4)nc(-c4ccc5cc(-c6ccccc6)ccc5c4)n3)c2)cc1]

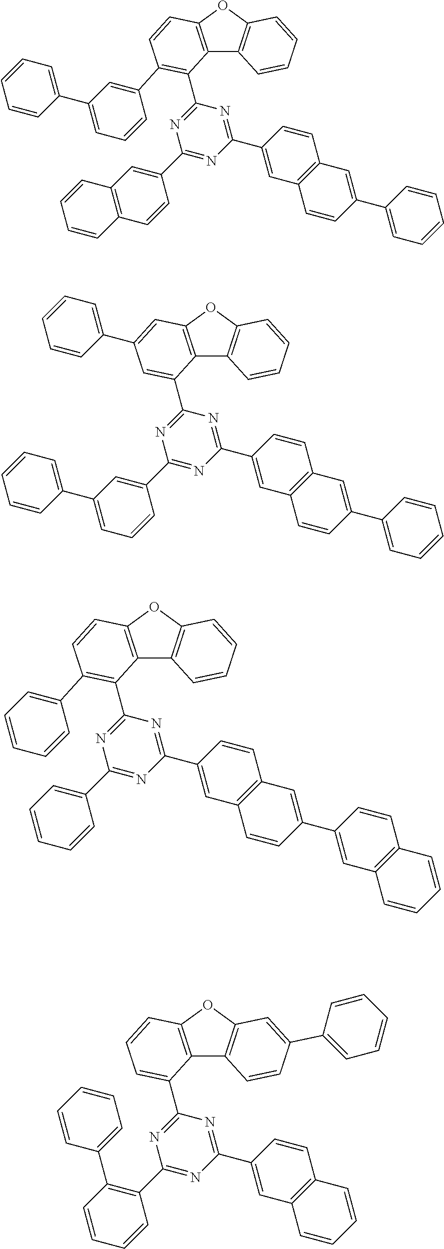 [SMILES: c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1 ; c1ccc(-c2cccc(-c3nc(-c4ccc5cc(-c6ccccc6)ccc5c4)nc(-c4cc(-c5ccccc5)cc5oc6ccccc6c45)n3)c2)cc1 ; c1ccc(-c2nc(-c3ccc4cc(-c5ccc6ccccc6c5)ccc4c3)nc(-c3c(-c4ccccc4)ccc4oc5ccccc5c34)n2)cc1 ; c1ccc(-c2ccc3c(c2)oc2cccc(-c4nc(-c5ccc6ccccc6c5)nc(-c5ccccc5-c5ccccc5)n4)c23)cc1]

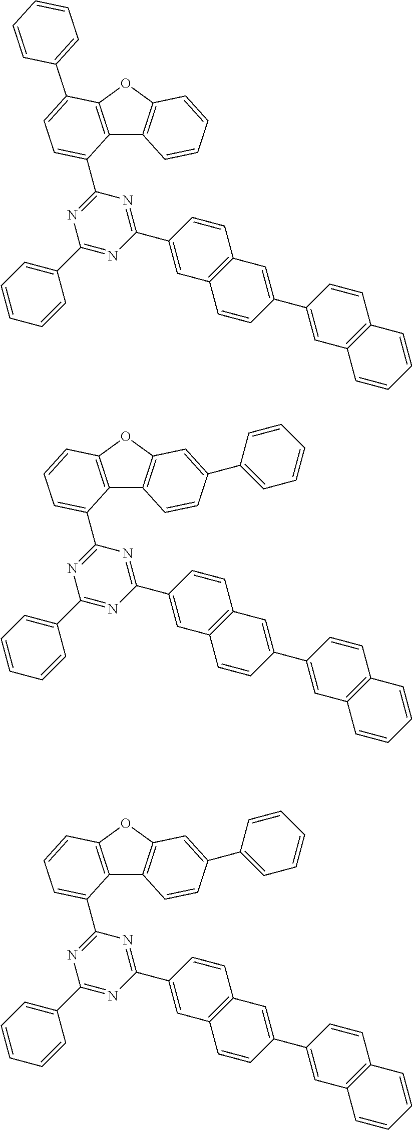 [SMILES: c1ccc(-c2nc(-c3ccc4cc(-c5ccc6ccccc6c5)ccc4c3)nc(-c3ccc(-c4ccccc4)c4oc5ccccc5c34)n2)cc1 ; c1ccc(-c2ccc3c(c2)oc2cccc(-c4nc(-c5ccccc5)nc(-c5ccc6cc(-c7ccc8ccccc8c7)ccc6c5)n4)c23)cc1 ; c1ccc(-c2ccc3c(c2)oc2cccc(-c4nc(-c5ccccc5)nc(-c5ccc6cc(-c7ccc8ccccc8c7)ccc6c5)n4)c23)cc1]

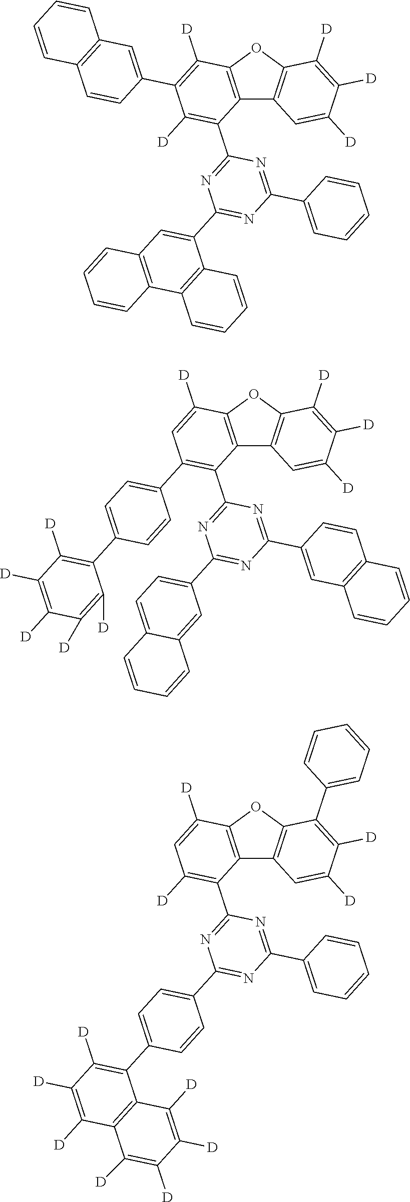 [SMILES: [2H]c1cc2c(oc3c([2H])c(-c4ccc5ccccc5c4)c([2H])c(-c4nc(-c5ccccc5)nc(-c5cc6ccccc6c6ccccc56)n4)c32)c([2H])c1[2H] ; [2H]c1cc2c(oc3c([2H])cc(-c4ccc(-c5c([2H])c([2H])c([2H])c([2H])c5[2H])cc4)c(-c4nc(-c5ccc6ccccc6c5)nc(-c5ccc6ccccc6c5)n4)c32)c([2H])c1[2H] ; [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H]]

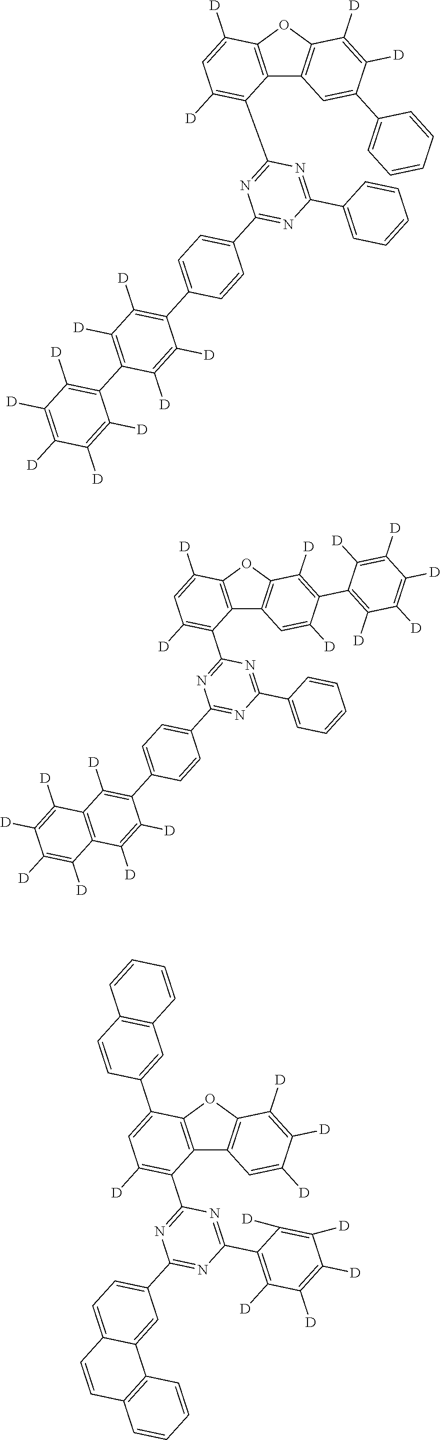 [SMILES: [2H]c1cc([2H])c2oc3c([2H])c([2H])c(-c4ccccc4)cc3c2c1-c1nc(-c2ccccc2)nc(-c2ccc(-c3c([2H])c([2H])c(-c4c([2H])c([2H])c([2H])c([2H])c4[2H])c([2H])c3[2H])cc2)n1 ; [2H]c1cc2c(oc3c([2H])cc([2H])c(-c4nc(-c5ccccc5)nc(-c5ccc(-c6c([2H])c([2H])c7c([2H])c([2H])c([2H])c([2H])c7c6[2H])cc5)n4)c32)c([2H])c1-c1c([2H])c([2H])c([2H])c([2H])c1[2H] ; [2H]c1cc2c(oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c32)c([2H])c1[2H]]

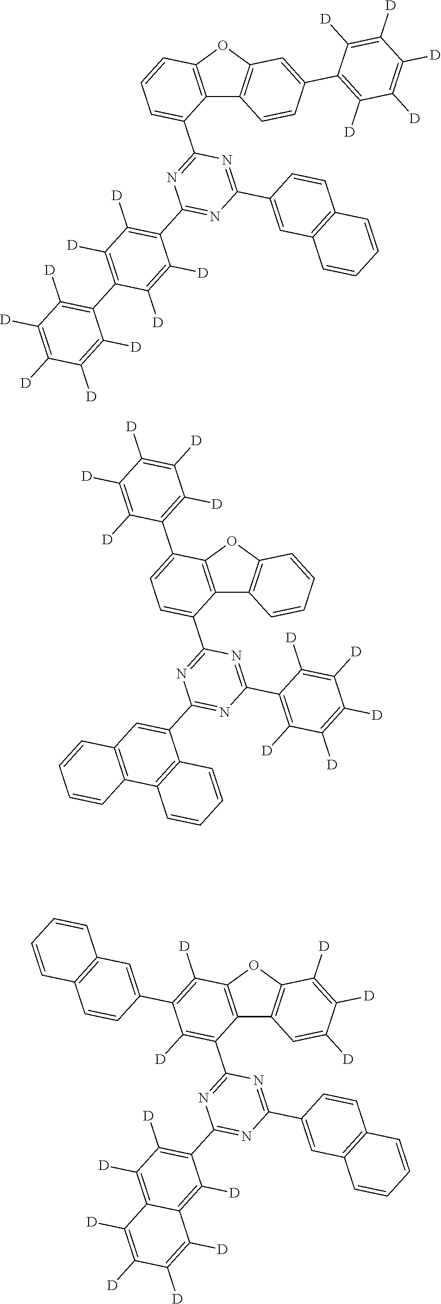 [SMILES: [2H]c1c([2H])c([2H])c(-c2ccc3c(c2)oc2cccc(-c4nc(-c5ccc6ccccc6c5)nc(-c5c([2H])c([2H])c(-c6c([2H])c([2H])c([2H])c([2H])c6[2H])c([2H])c5[2H])n4)c23)c([2H])c1[2H] ; [2H]c1c([2H])c([2H])c(-c2nc(-c3cc4ccccc4c4ccccc34)nc(-c3ccc(-c4c([2H])c([2H])c([2H])c([2H])c4[2H])c4oc5ccccc5c34)n2)c([2H])c1[2H] ; [2H]c1cc2c(oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c32)c([2H])c1[2H]]

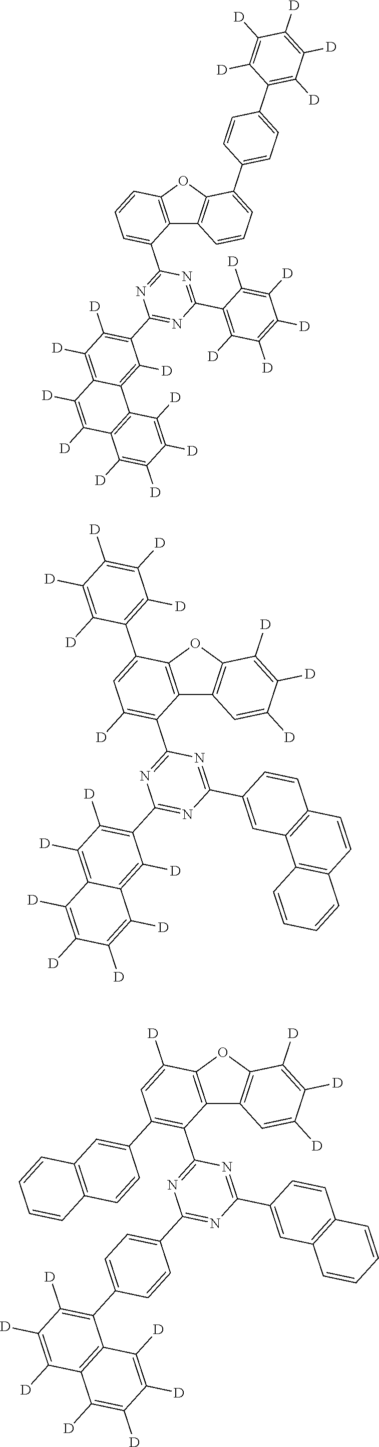

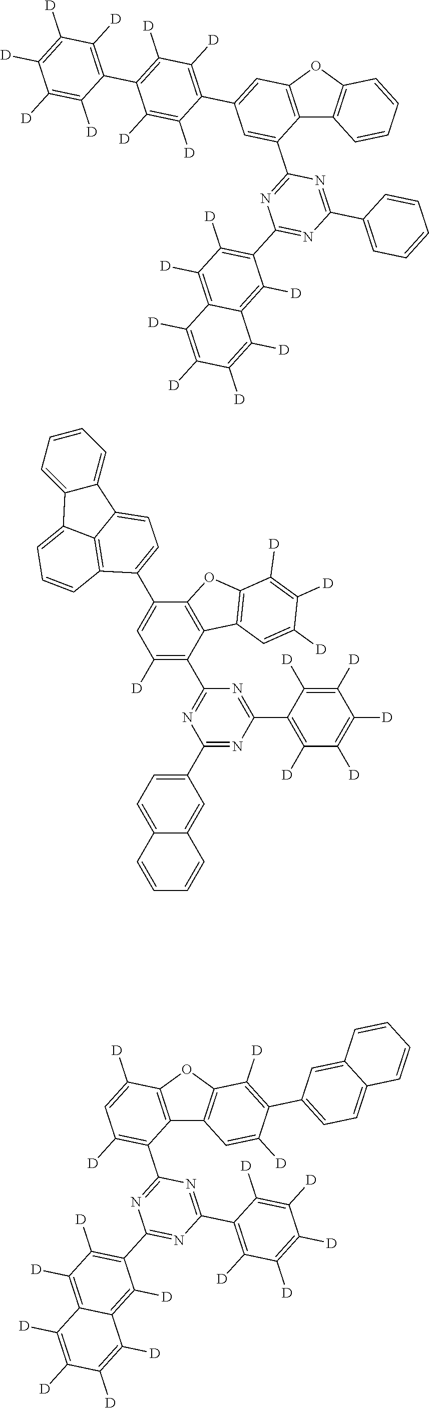 [SMILES: [2H]c1c([2H])c([2H])c(-c2c([2H])c([2H])c(-c3cc(-c4nc(-c5ccccc5)nc(-c5c([2H])c([2H])c6c([2H])c([2H])c([2H])c([2H])c6c5[2H])n4)c4c(c3)oc3ccccc34)c([2H])c2[2H])c([2H])c1[2H] ; [2H]c1cc2c(oc3c(-c4ccc5c6c(cccc46)-c4ccccc4-5)cc([2H])c(-c4nc(-c5ccc6ccccc6c5)nc(-c5c([2H])c([2H])c([2H])c([2H])c5[2H])n4)c32)c([2H])c1[2H] ; [2H]c1cc2c(oc3c([2H])cc([2H])c(-c4nc(-c5c([2H])c([2H])c([2H])c([2H])c5[2H])nc(-c5c([2H])c([2H])c6c([2H])c([2H])c([2H])c([2H])c6c5[2H])n4)c32)c([2H])c1-c1ccc2ccccc2c1]

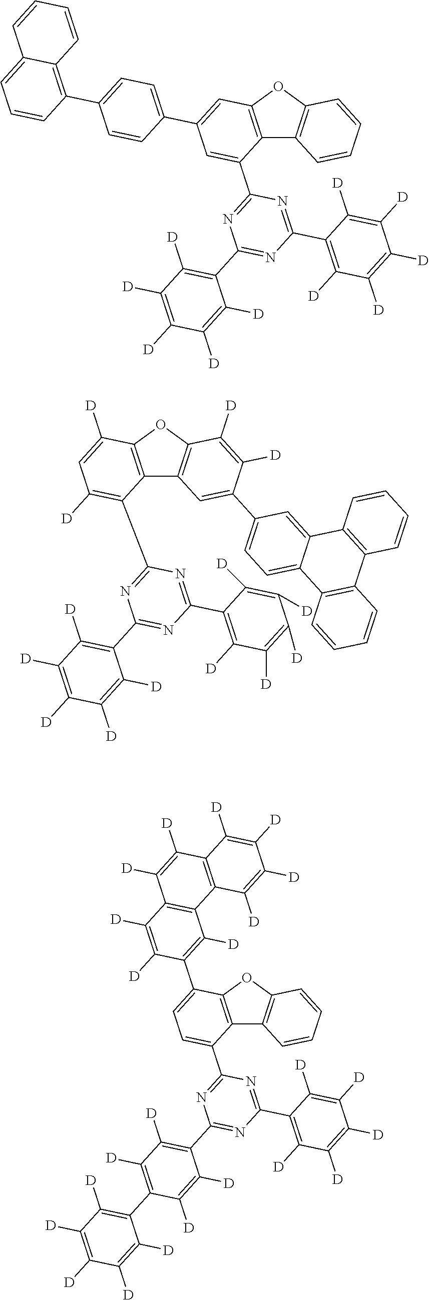

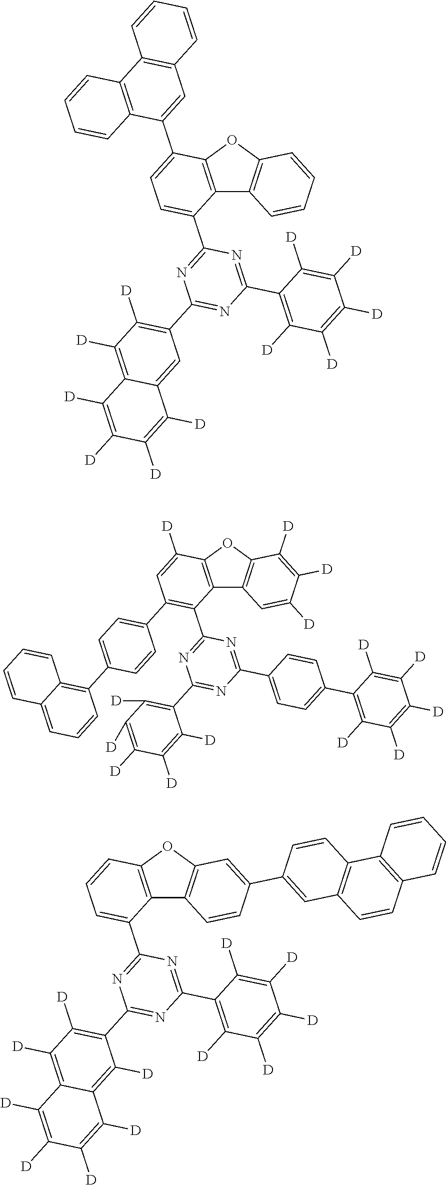 [SMILES: [2H]c1c([2H])c([2H])c(-c2nc(-c3cc4c([2H])c([2H])c([2H])c([2H])c4c([2H])c3[2H])nc(-c3ccc(-c4cc5ccccc5c5ccccc45)c4oc5ccccc5c34)n2)c([2H])c1[2H] ; [2H]c1cc2c(oc3c([2H])cc(-c4ccc(-c5cccc6ccccc56)cc4)c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H]]

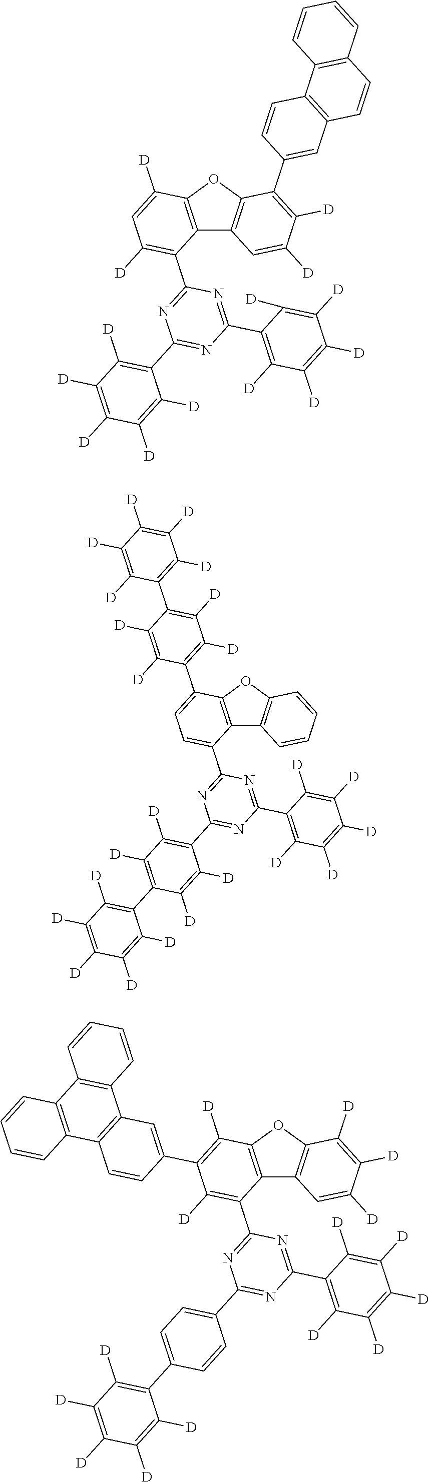

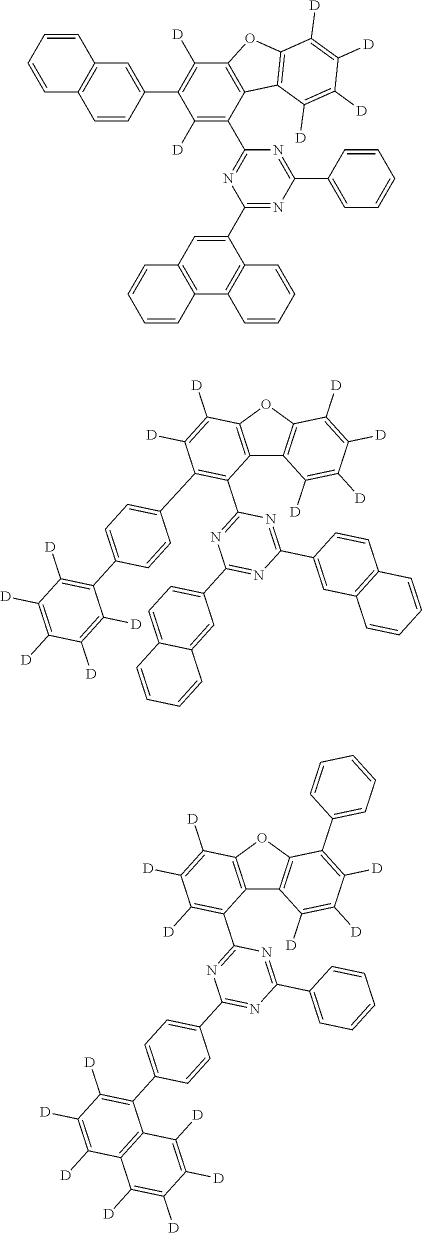 [SMILES: [2H]c1c([2H])c([2H])c2c(oc3c([2H])c(-c4ccc5ccccc5c4)c([2H])c(-c4nc(-c5ccccc5)nc(-c5cc6ccccc6c6ccccc56)n4)c32)c1[2H] ; [2H]c1c([2H])c([2H])c(-c2ccc(-c3c([2H])c([2H])c4oc5c([2H])c([2H])c([2H])c([2H])c5c4c3-c3nc(-c4ccc5ccccc5c4)nc(-c4ccc5ccccc5c4)n3)cc2)c([2H])c1[2H] ; [2H]c1c([2H])c(-c2nc(-c3ccccc3)nc(-c3ccc(-c4c([2H])c([2H])c([2H])c5c([2H])c([2H])c([2H])c([2H])c45)cc3)n2)c2c(oc3c(-c4ccccc4)c([2H])c([2H])c([2H])c32)c1[2H]]

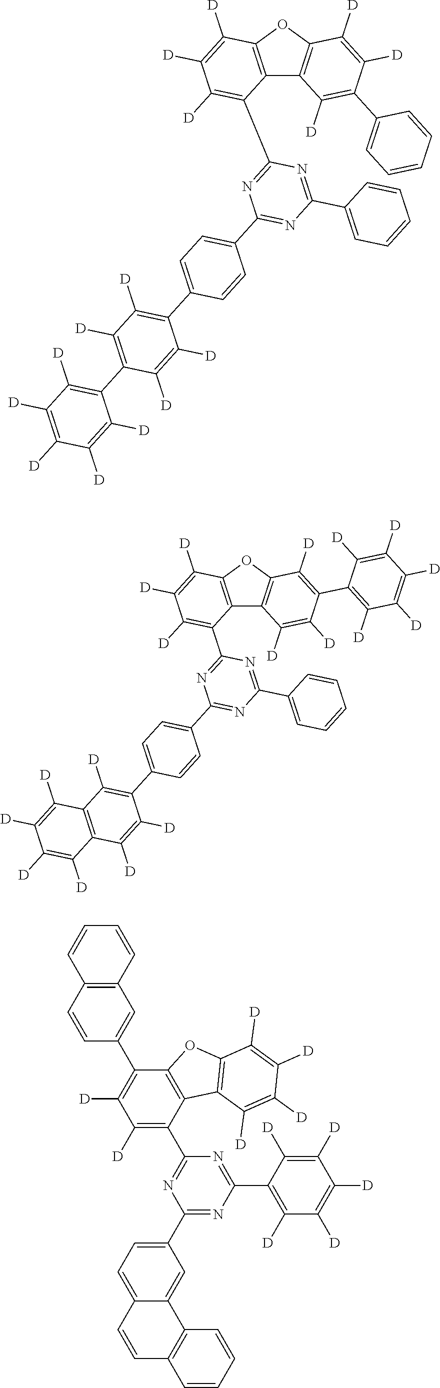

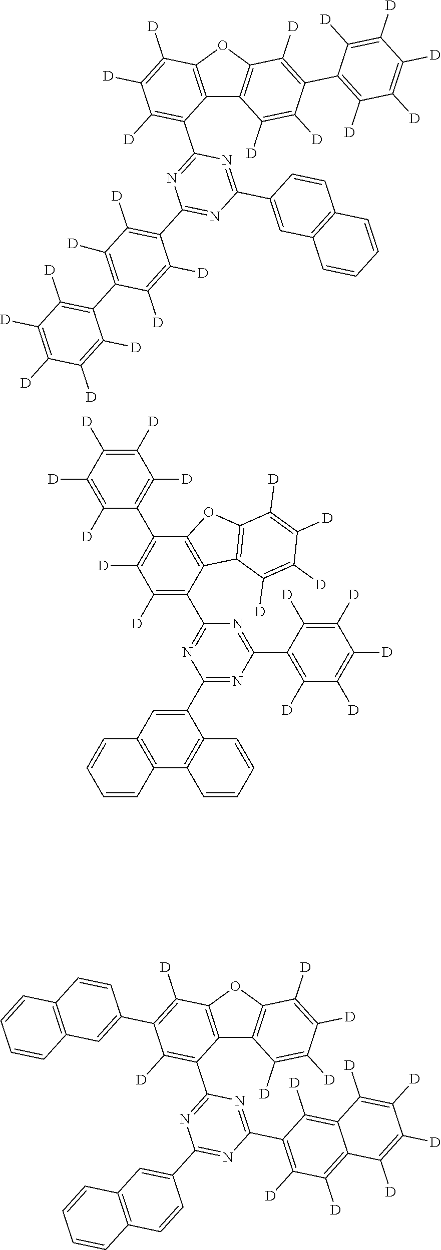

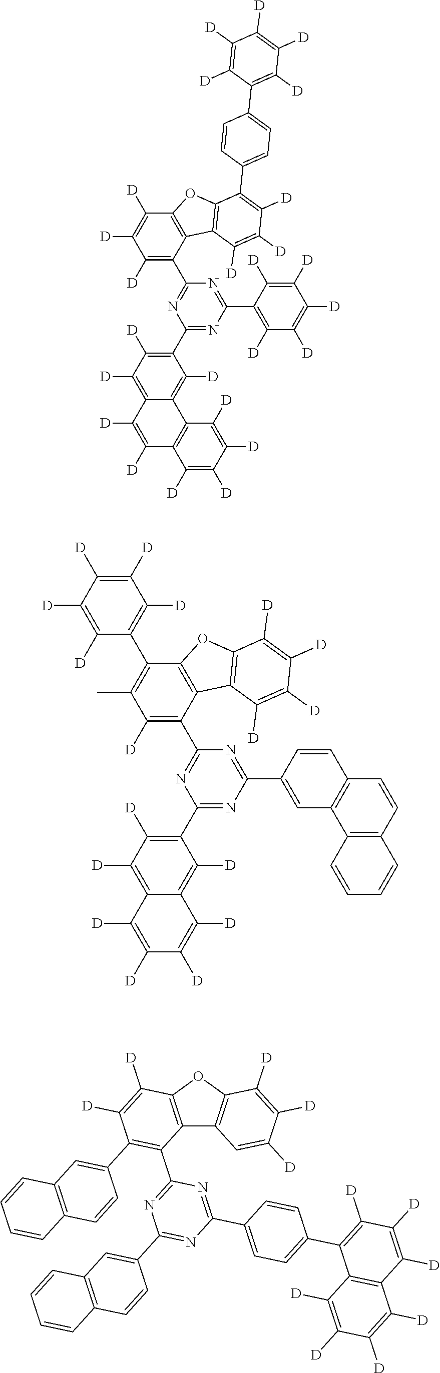

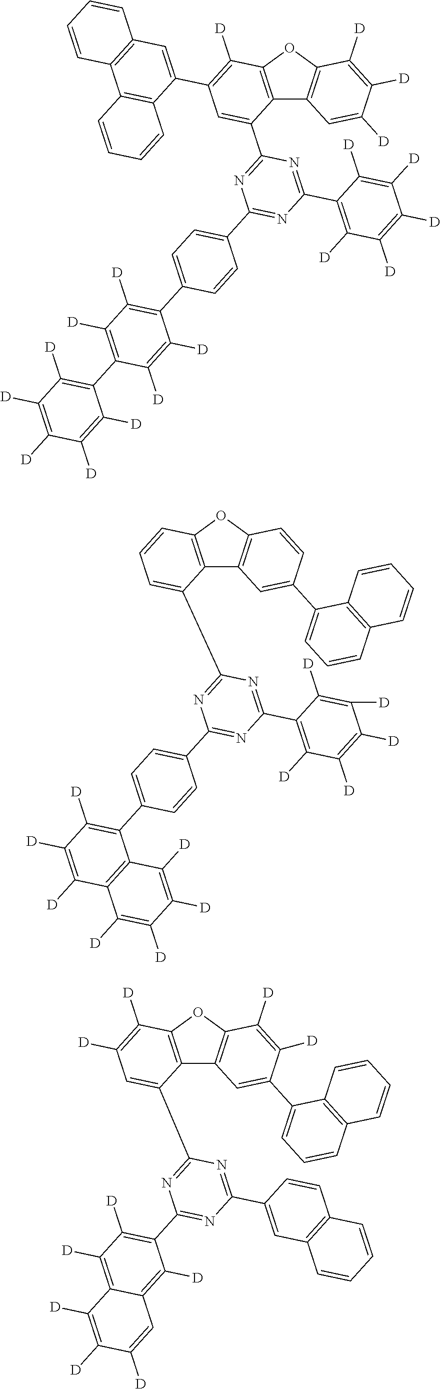 [SMILES: [2H]c1cc2c(oc3c([2H])c(-c4cc5ccccc5c5ccccc45)cc(-c4nc(-c5ccc(-c6c([2H])c([2H])c(-c7c([2H])c([2H])c([2H])c([2H])c7[2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3ccc(-c4c([2H])c([2H])c([2H])c5c([2H])c([2H])c([2H])c([2H])c45)cc3)nc(-c3cccc4oc5ccc(-c6cccc7ccccc67)cc5c34)n2)c([2H])c1[2H] ; [2H]c1cc2c([2H])c(-c3nc(-c4ccc5ccccc5c4)nc(-c4cc([2H])c([2H])c5oc6c([2H])c([2H])c(-c7cccc8ccccc78)cc6c45)n3)c([2H])c([2H])c2c([2H])c1[2H]]

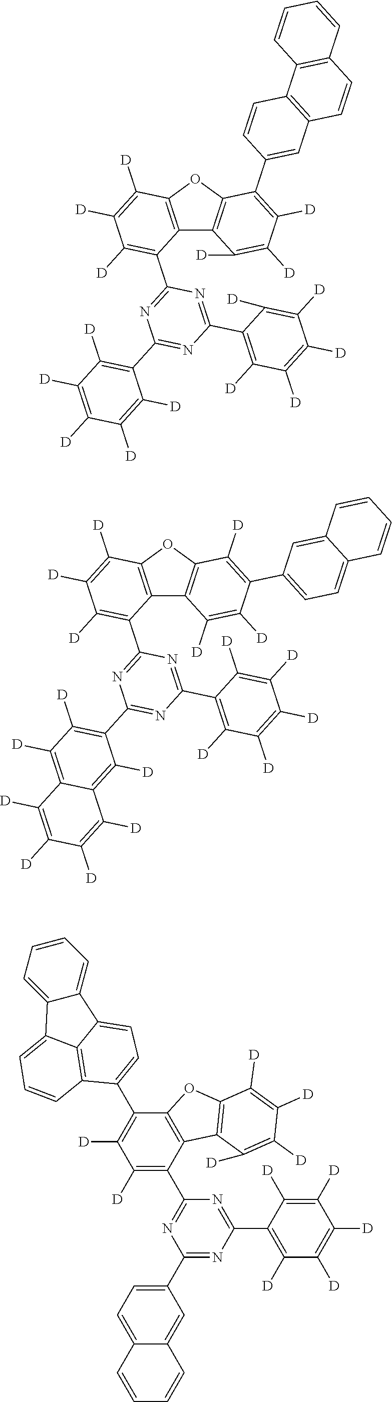

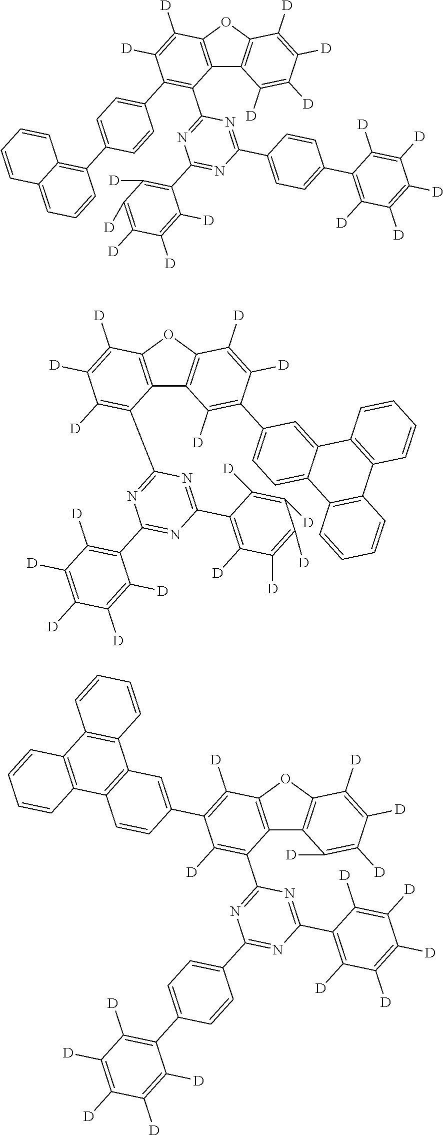

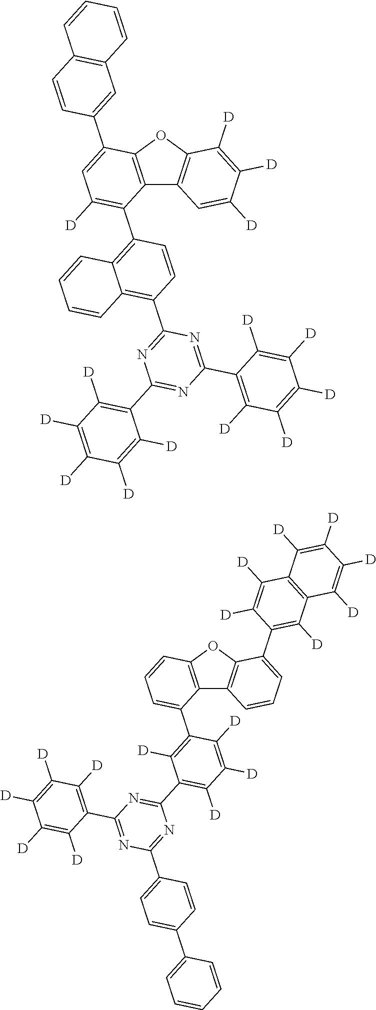 [SMILES: [2H]c1cc2c(oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c32)c([2H])c1[2H] ; [2H]c1c([2H])c([2H])c(-c2nc(-c3ccc(-c4ccccc4)cc3)nc(-c3c([2H])c([2H])c([2H])c(-c4cccc5oc6c(-c7c([2H])c([2H])c8c([2H])c([2H])c([2H])c([2H])c8c7[2H])cccc6c45)c3[2H])n2)c([2H])c1[2H]]

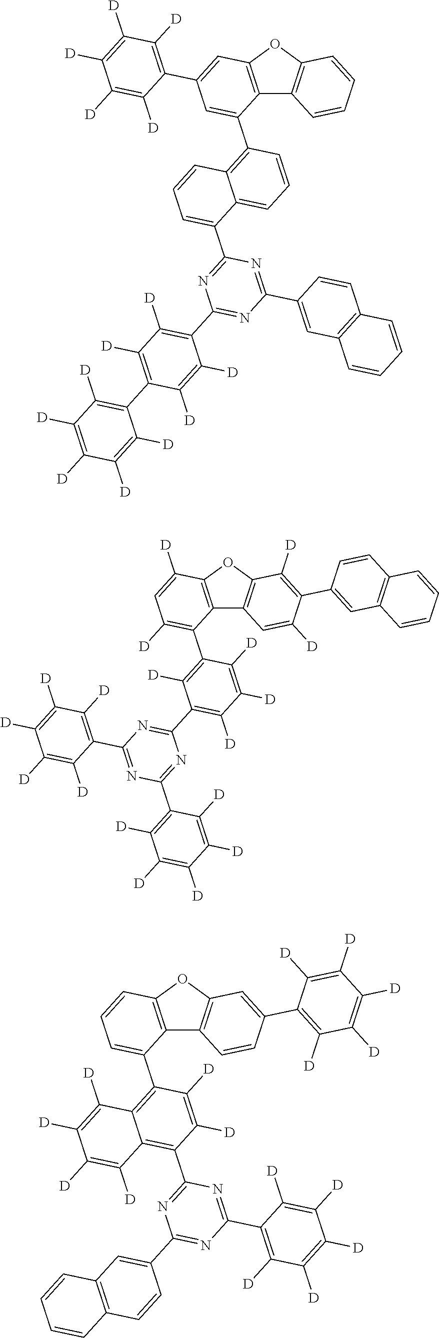

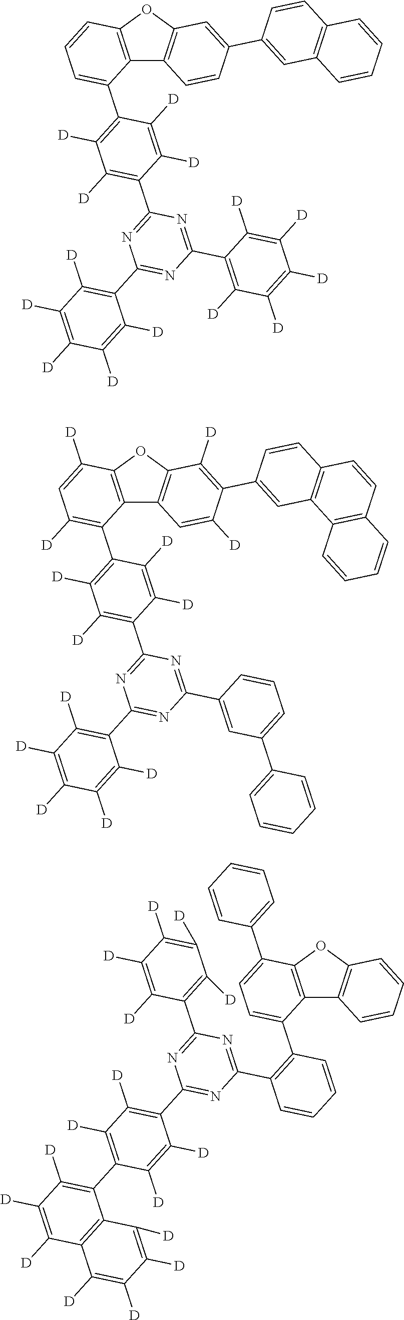

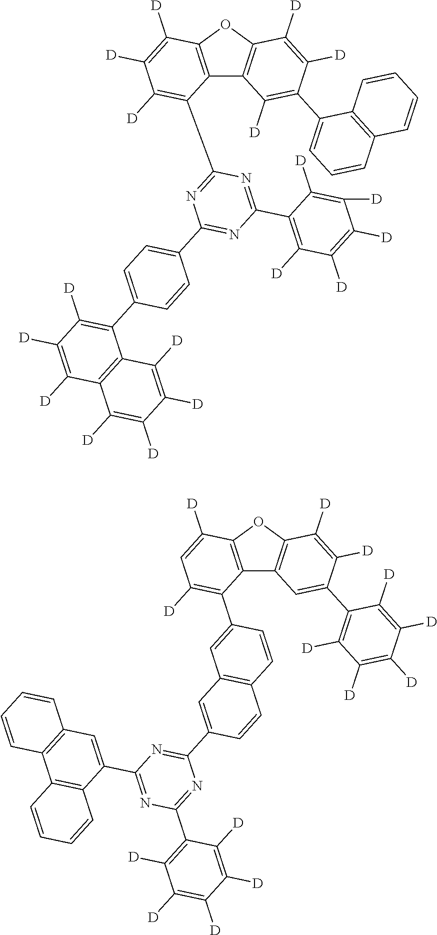 [SMILES: [2H]c1c([2H])c([2H])c(-c2nc(-c3ccc(-c4c([2H])c([2H])c([2H])c5c([2H])c([2H])c([2H])c([2H])c45)cc3)nc(-c3c([2H])c([2H])c([2H])c4oc5c([2H])c([2H])c(-c6cccc7ccccc67)c([2H])c5c34)n2)c([2H])c1[2H] ; [2H]c1cc([2H])c2oc3c([2H])c([2H])c(-c4c([2H])c([2H])c([2H])c([2H])c4[2H])cc3c2c1-c1ccc2ccc(-c3nc(-c4c([2H])c([2H])c([2H])c([2H])c4[2H])nc(-c4cc5ccccc5c5ccccc45)n3)cc2c1]

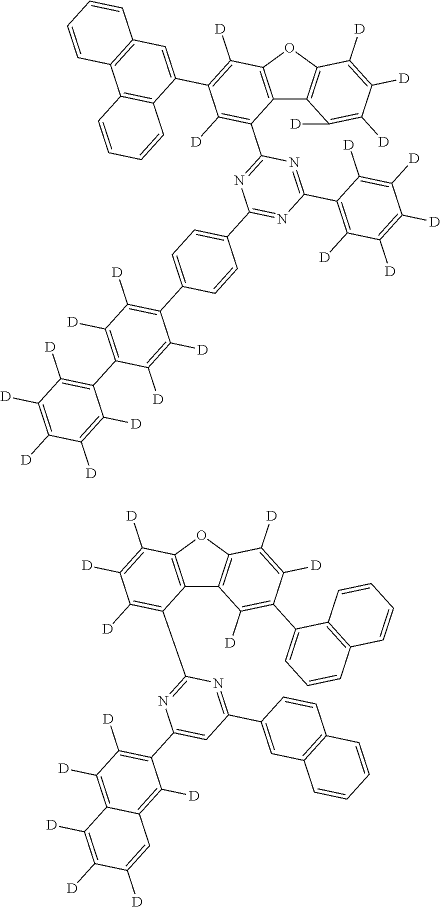 [SMILES: [2H]c1c([2H])c([2H])c(-c2nc(-c3ccc(-c4c([2H])c([2H])c(-c5c([2H])c([2H])c([2H])c([2H])c5[2H])c([2H])c4[2H])cc3)nc(-c3c([2H])c(-c4cc5ccccc5c5ccccc45)c([2H])c4oc5c([2H])c([2H])c([2H])c([2H])c5c34)n2)c([2H])c1[2H] ; [2H]c1cc2c([2H])c(-c3cc(-c4ccc5ccccc5c4)nc(-c4c([2H])c([2H])c([2H])c5oc6c([2H])c([2H])c(-c7cccc8ccccc78)c([2H])c6c45)n3)c([2H])c([2H])c2c([2H])c1[2H]]

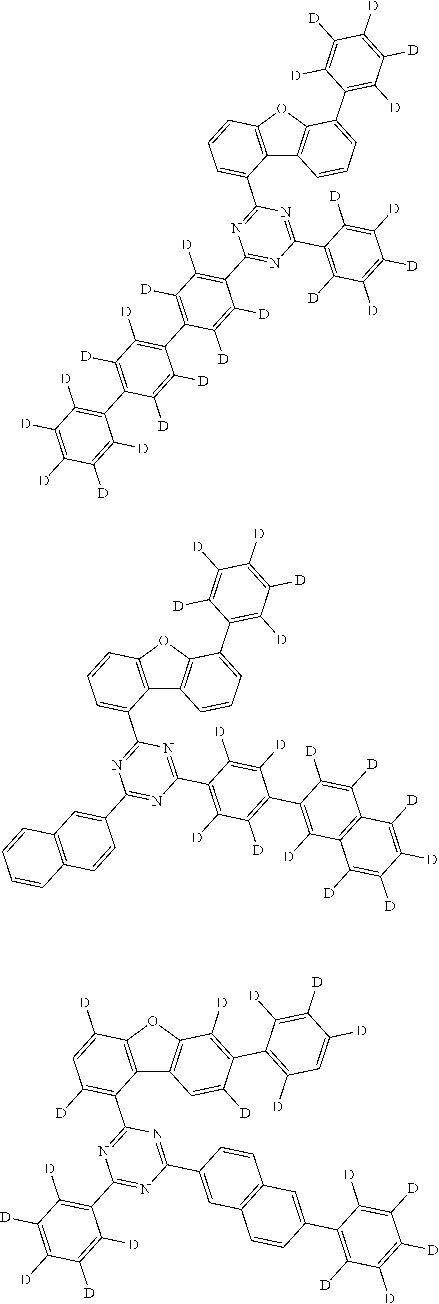

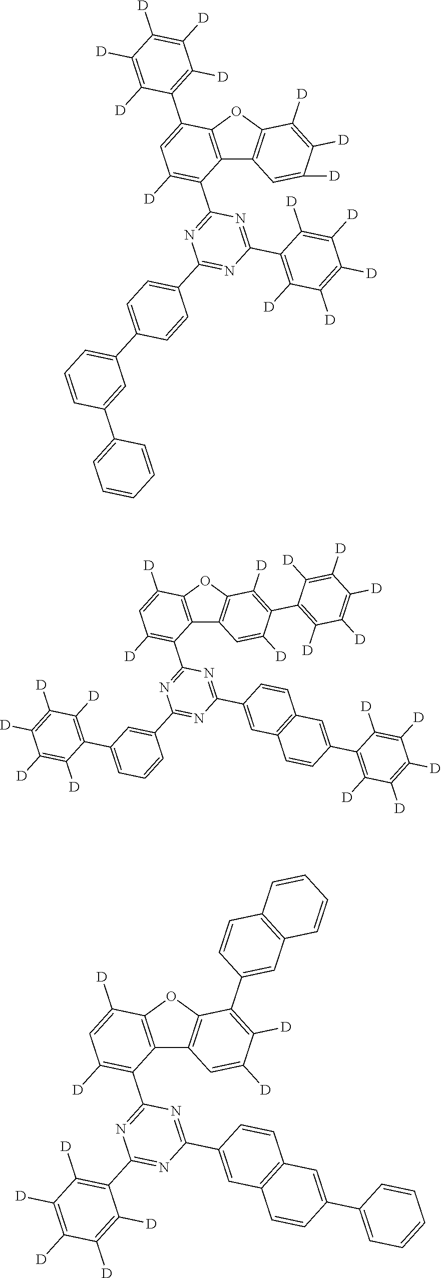

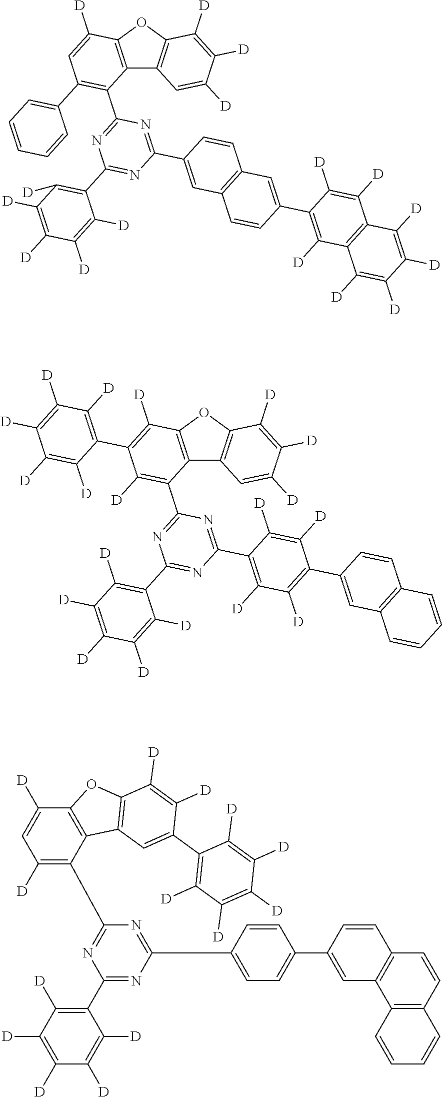

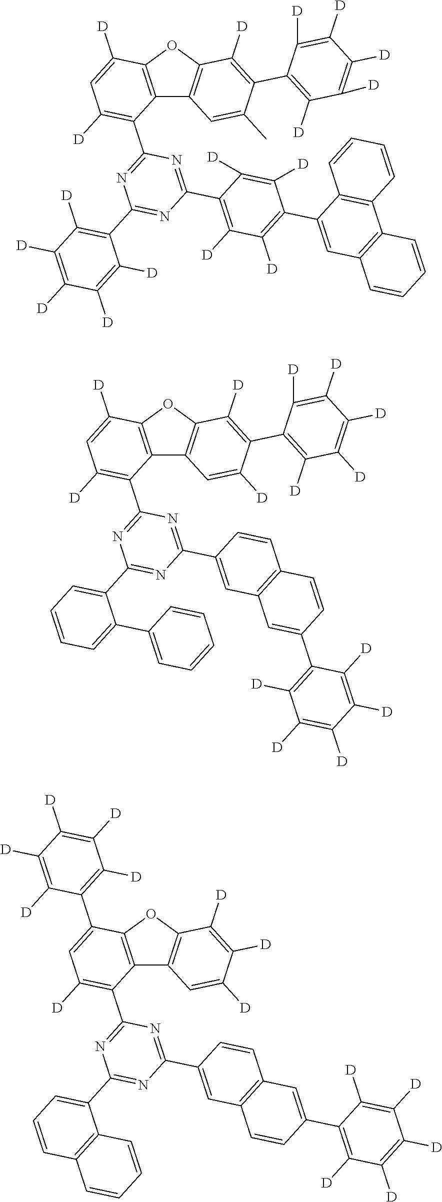

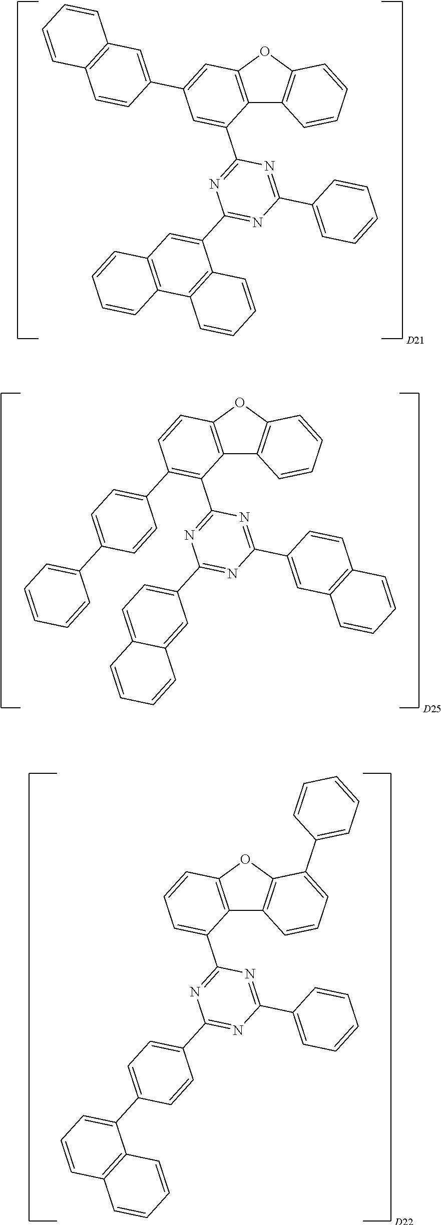

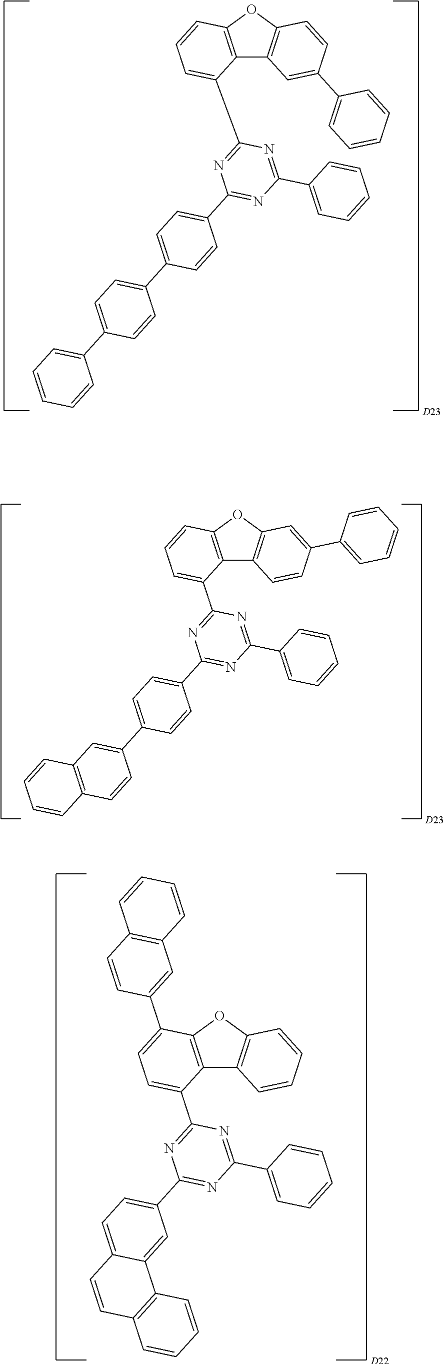

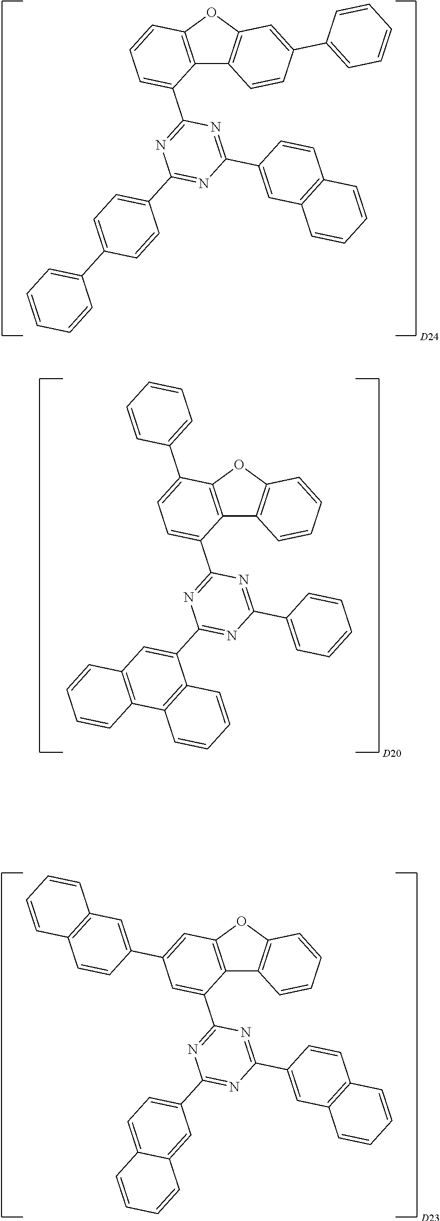

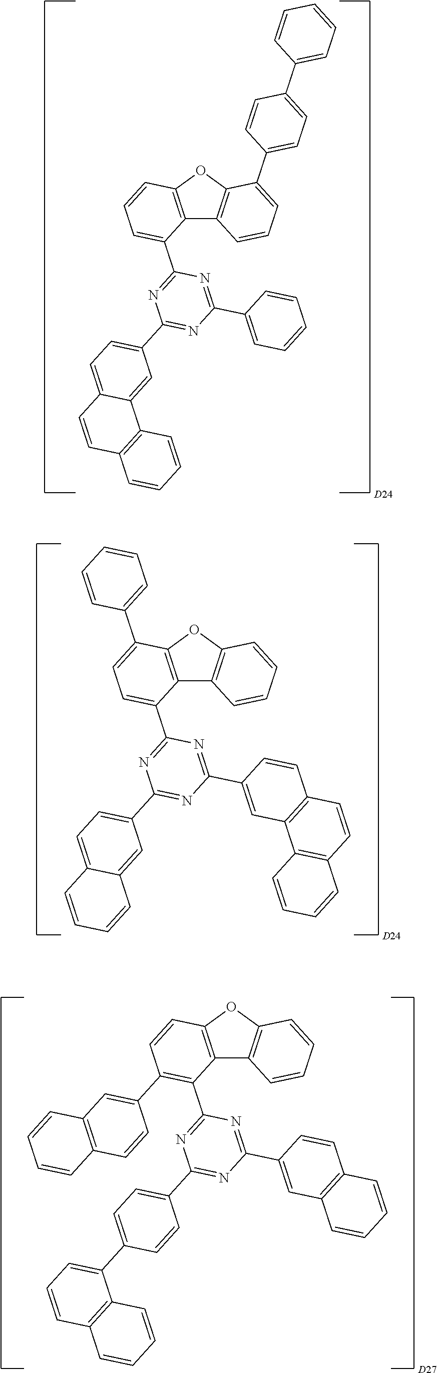

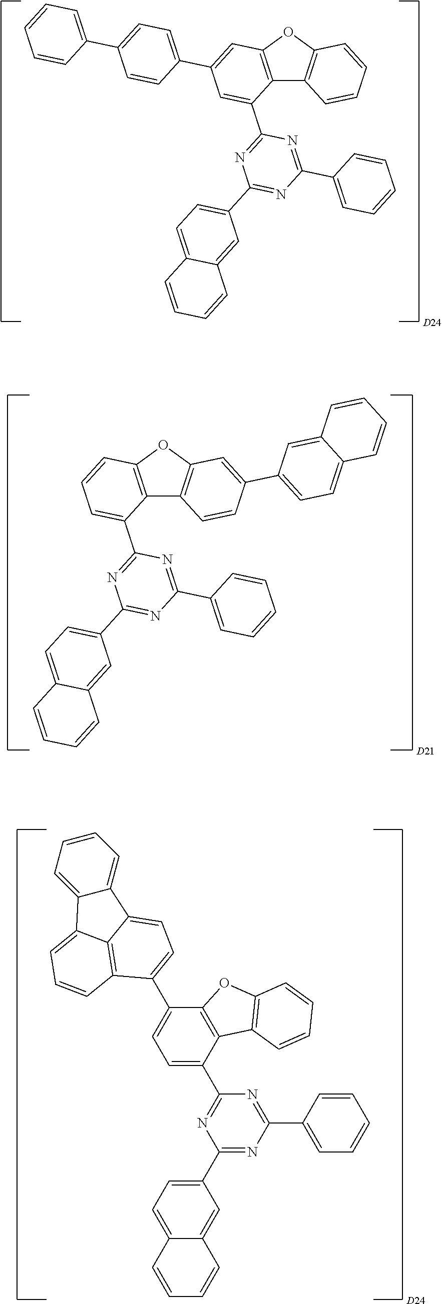

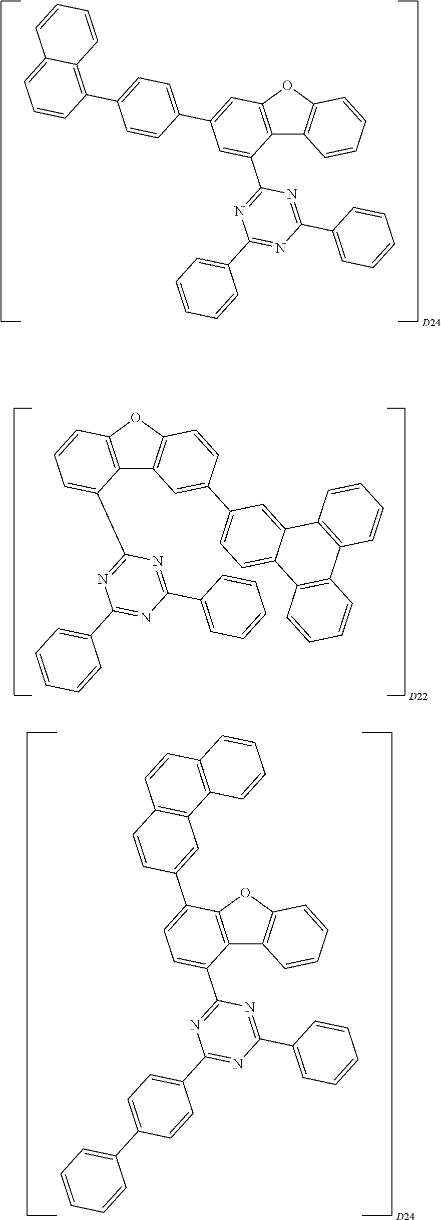

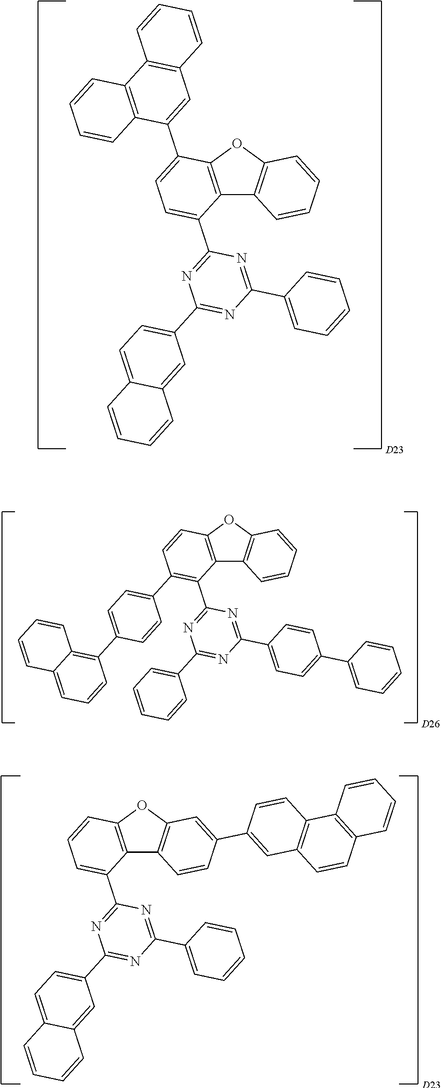

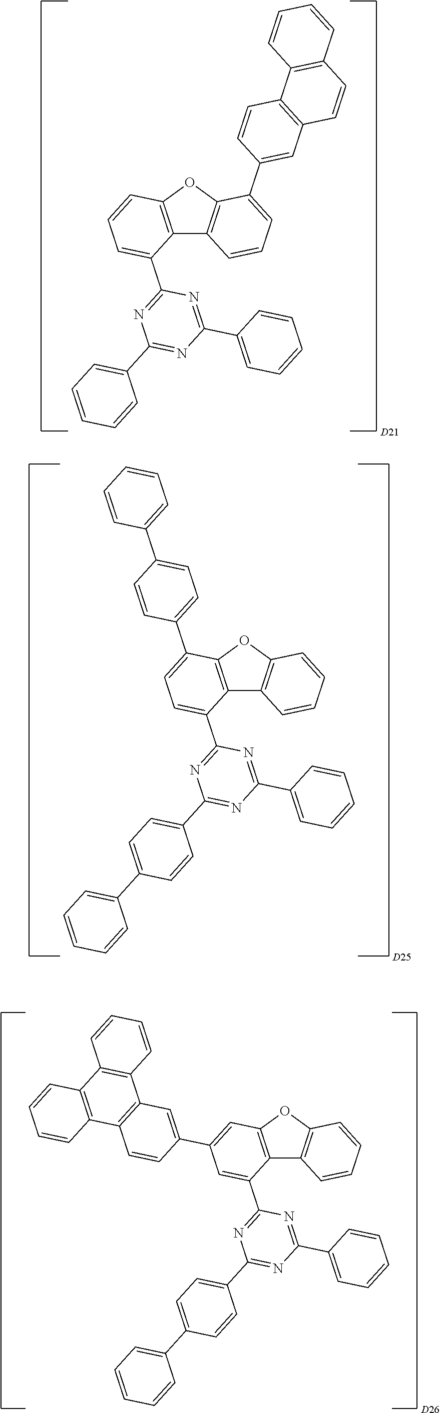

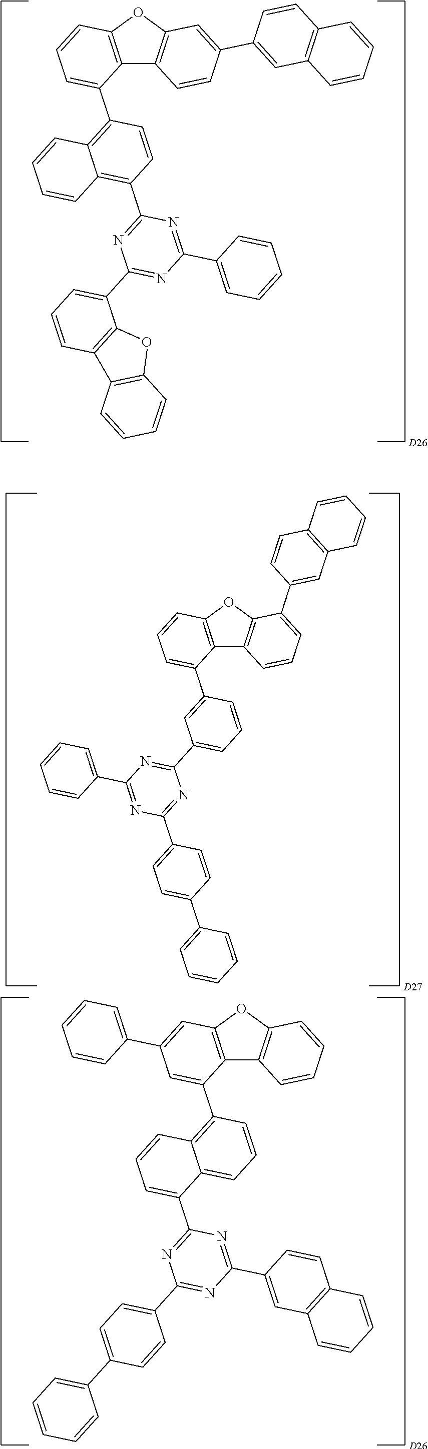

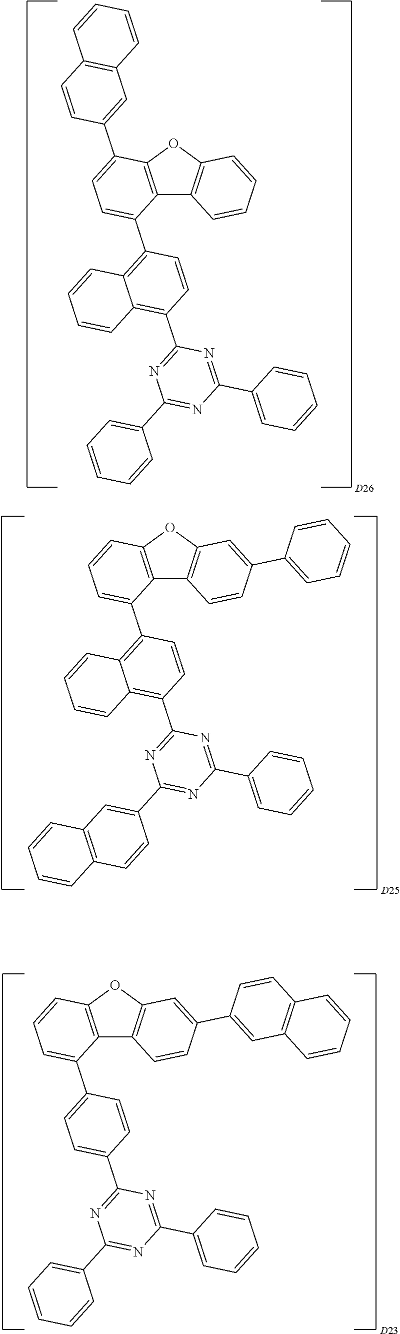

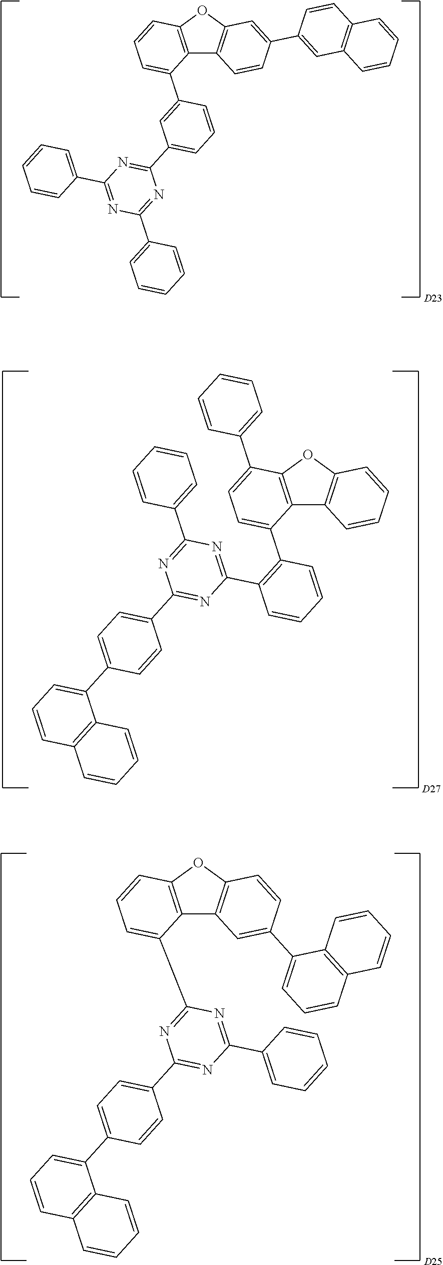

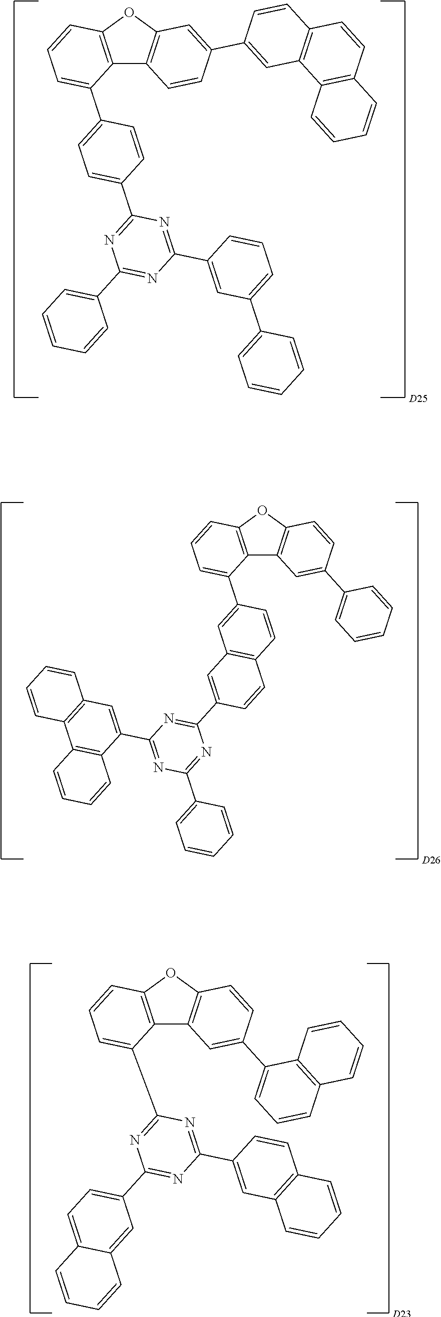

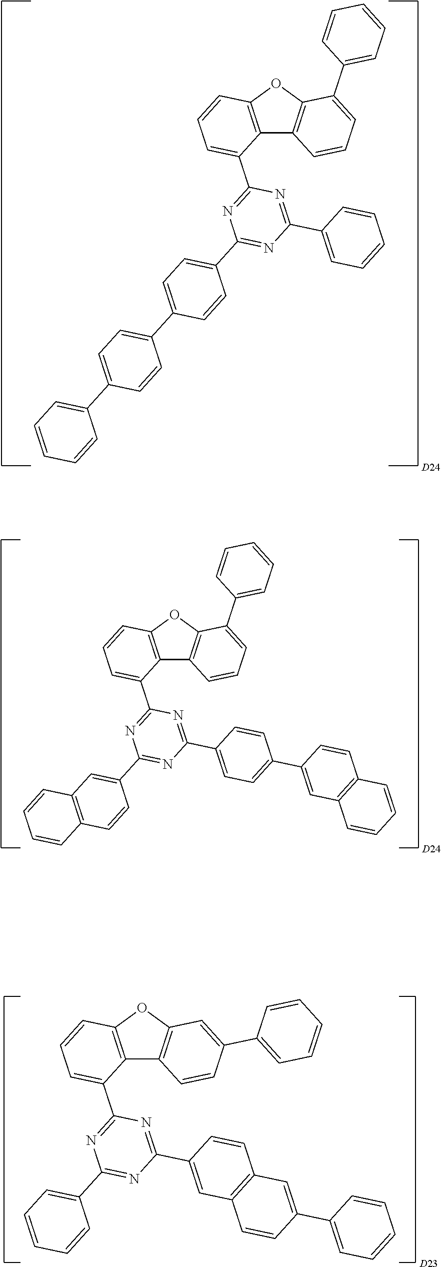

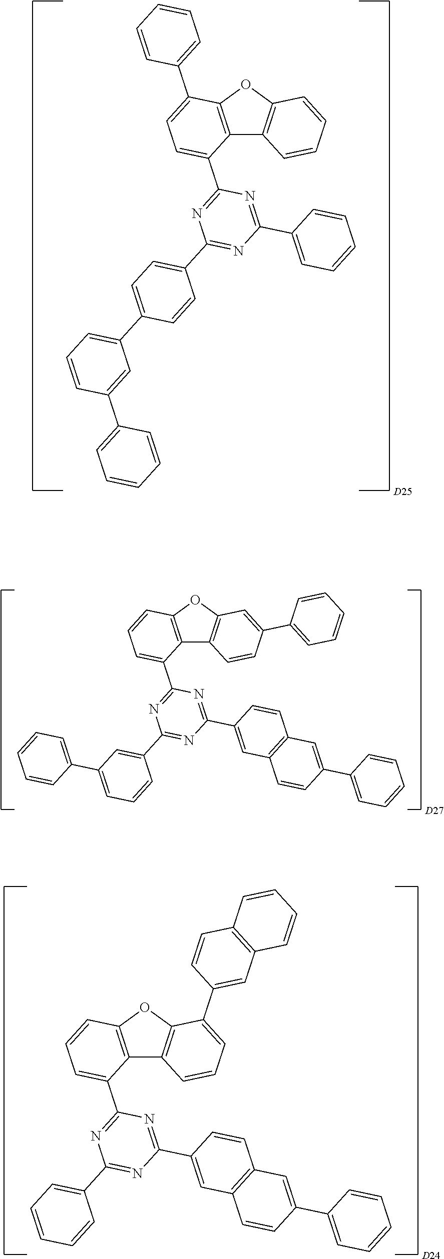

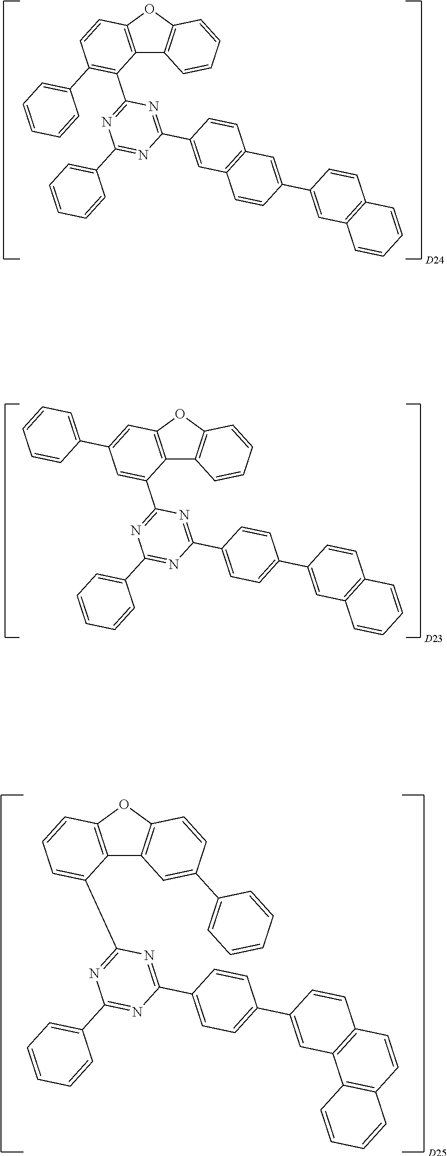

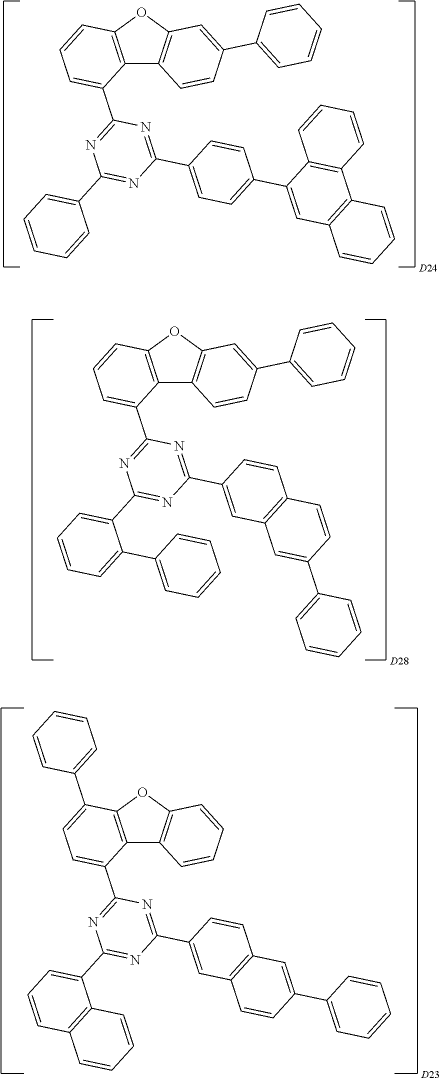

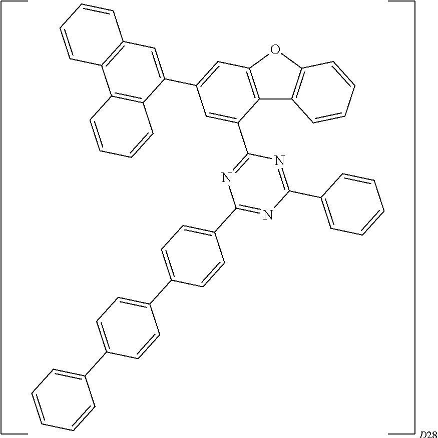

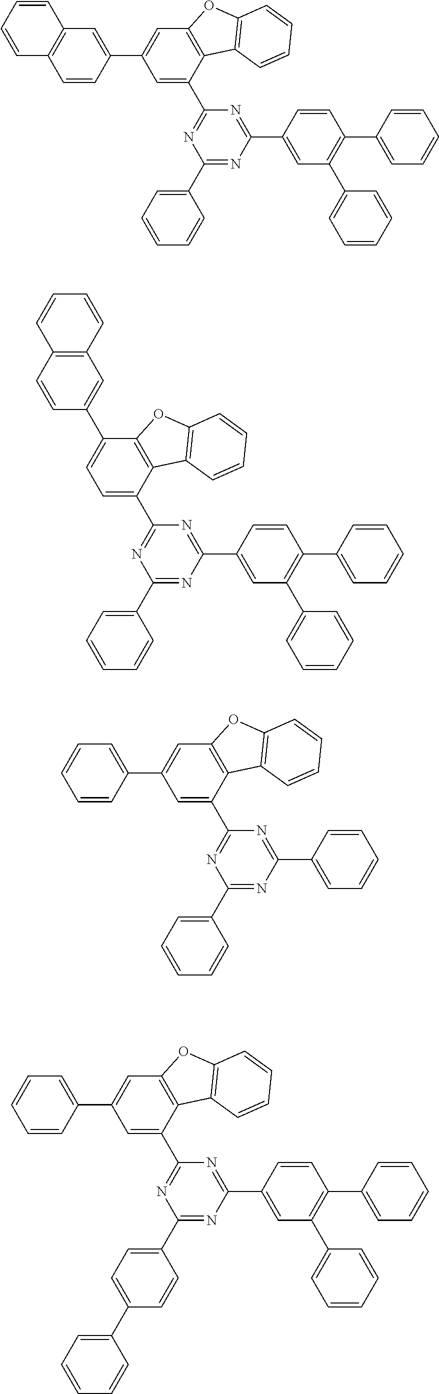 [SMILES: c1ccc(-c2nc(-c3ccc(-c4ccccc4)c(-c4ccccc4)c3)nc(-c3cc(-c4ccc5ccccc5c4)cc4oc5ccccc5c34)n2)cc1 ; c1ccc(-c2nc(-c3ccc(-c4ccccc4)c(-c4ccccc4)c3)nc(-c3ccc(-c4ccc5ccccc5c4)c4oc5ccccc5c34)n2)cc1 ; c1ccc(-c2cc(-c3nc(-c4ccccc4)nc(-c4ccccc4)n3)c3c(c2)oc2ccccc23)cc1 ; c1ccc(-c2ccc(-c3nc(-c4ccc(-c5ccccc5)c(-c5ccccc5)c4)nc(-c4cc(-c5ccccc5)cc5oc6ccccc6c45)n3)cc2)cc1]

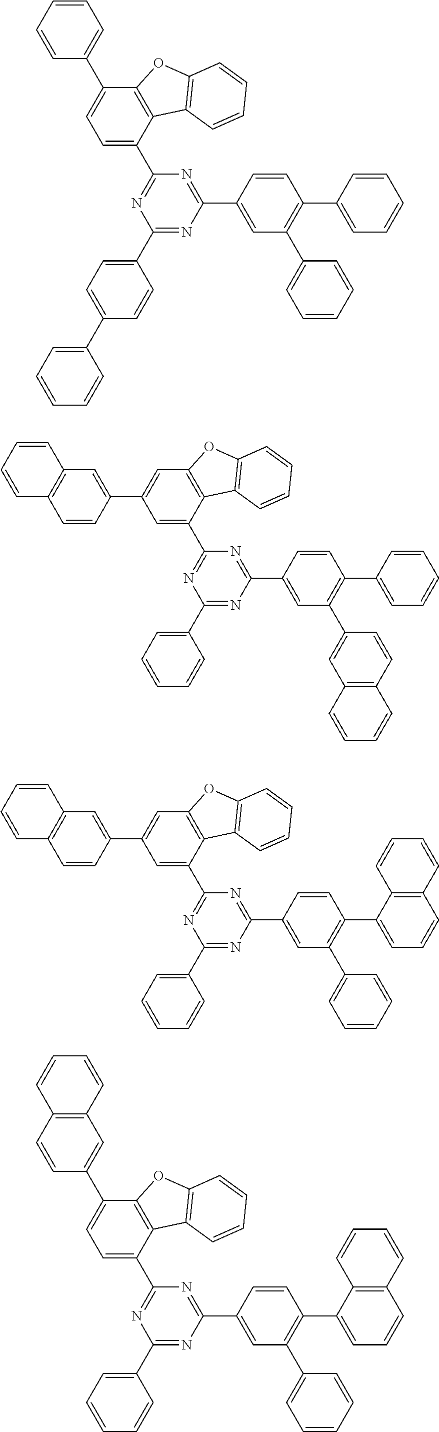

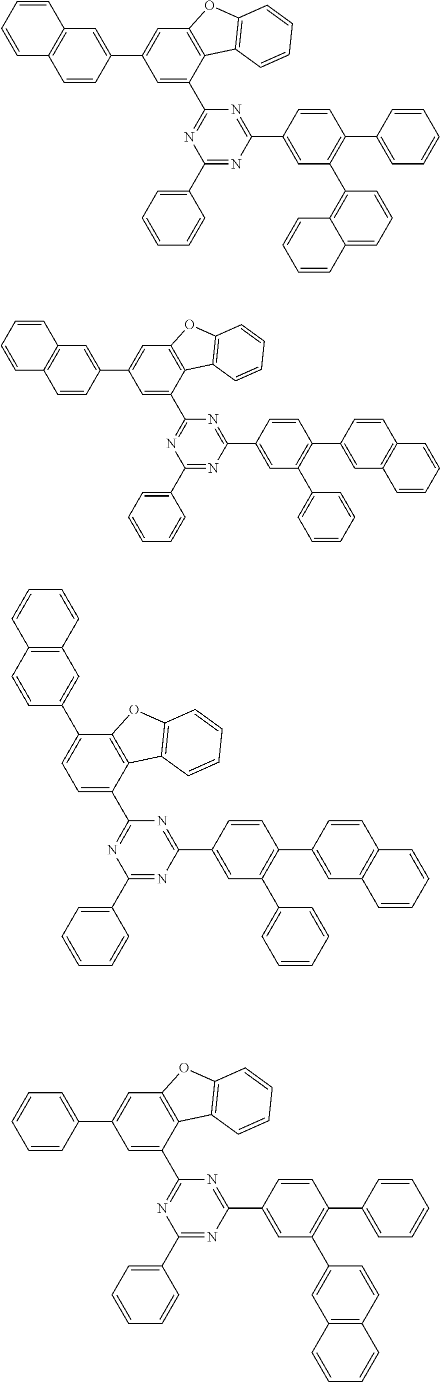 [SMILES: c1ccc(-c2nc(-c3ccc(-c4ccccc4)c(-c4cccc5ccccc45)c3)nc(-c3cc(-c4ccc5ccccc5c4)cc4oc5ccccc5c34)n2)cc1 ; c1ccc(-c2nc(-c3ccc(-c4ccc5ccccc5c4)c(-c4ccccc4)c3)nc(-c3cc(-c4ccc5ccccc5c4)cc4oc5ccccc5c34)n2)cc1 ; c1ccc(-c2nc(-c3ccc(-c4ccc5ccccc5c4)c(-c4ccccc4)c3)nc(-c3ccc(-c4ccc5ccccc5c4)c4oc5ccccc5c34)n2)cc1 ; c1ccc(-c2cc(-c3nc(-c4ccccc4)nc(-c4ccc(-c5ccccc5)c(-c5ccc6ccccc6c5)c4)n3)c3c(c2)oc2ccccc23)cc1]

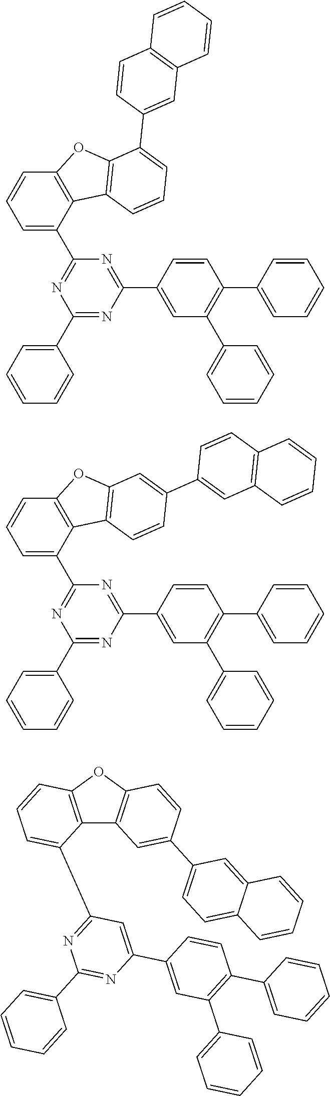

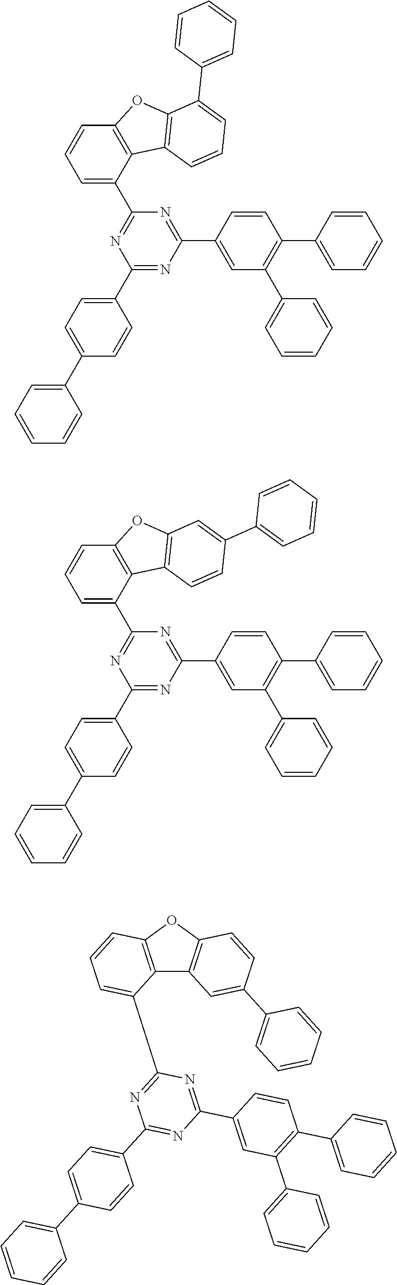 [SMILES: c1ccc(-c2ccc(-c3nc(-c4ccc(-c5ccccc5)c(-c5ccccc5)c4)nc(-c4cccc5oc6c(-c7ccccc7)cccc6c45)n3)cc2)cc1 ; c1ccc(-c2ccc(-c3nc(-c4ccc(-c5ccccc5)c(-c5ccccc5)c4)nc(-c4cccc5oc6cc(-c7ccccc7)ccc6c45)n3)cc2)cc1 ; c1ccc(-c2ccc(-c3nc(-c4ccc(-c5ccccc5)c(-c5ccccc5)c4)nc(-c4cccc5oc6ccc(-c7ccccc7)cc6c45)n3)cc2)cc1]

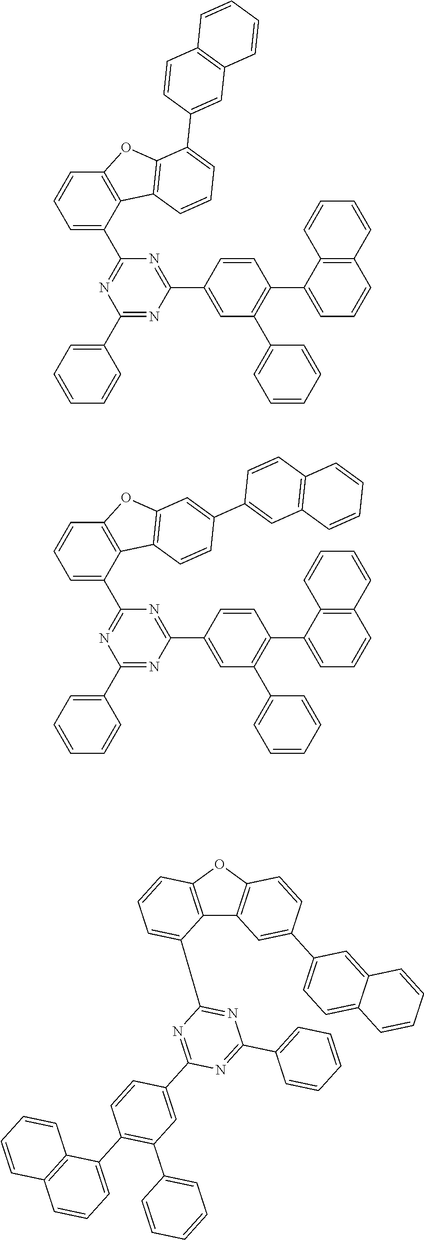 [SMILES: c1ccc(-c2nc(-c3ccc(-c4cccc5ccccc45)c(-c4ccccc4)c3)nc(-c3cccc4oc5c(-c6ccc7ccccc7c6)cccc5c34)n2)cc1 ; c1ccc(-c2nc(-c3ccc(-c4cccc5ccccc45)c(-c4ccccc4)c3)nc(-c3cccc4oc5cc(-c6ccc7ccccc7c6)ccc5c34)n2)cc1 ; c1ccc(-c2nc(-c3ccc(-c4cccc5ccccc45)c(-c4ccccc4)c3)nc(-c3cccc4oc5ccc(-c6ccc7ccccc7c6)cc5c34)n2)cc1]

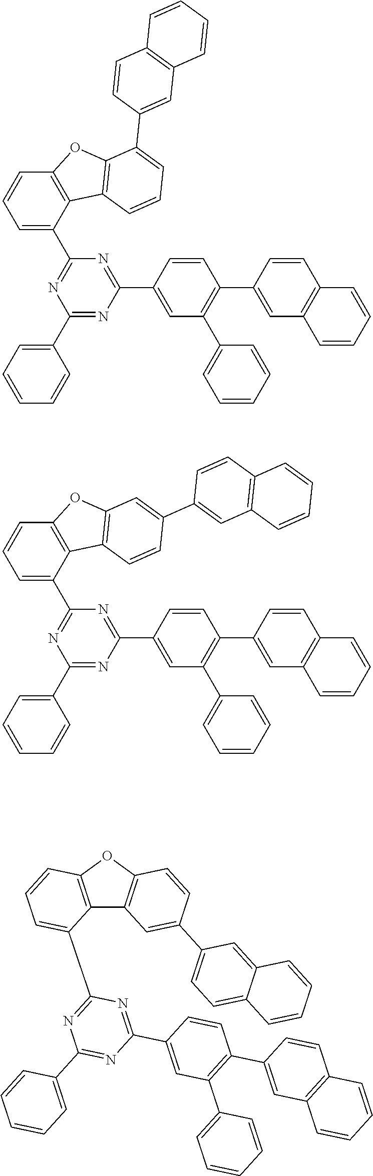 [SMILES: c1ccc(-c2nc(-c3ccc(-c4ccc5ccccc5c4)c(-c4ccccc4)c3)nc(-c3cccc4oc5c(-c6ccc7ccccc7c6)cccc5c34)n2)cc1 ; c1ccc(-c2nc(-c3ccc(-c4ccc5ccccc5c4)c(-c4ccccc4)c3)nc(-c3cccc4oc5cc(-c6ccc7ccccc7c6)ccc5c34)n2)cc1 ; c1ccc(-c2nc(-c3ccc(-c4ccc5ccccc5c4)c(-c4ccccc4)c3)nc(-c3cccc4oc5ccc(-c6ccc7ccccc7c6)cc5c34)n2)cc1]

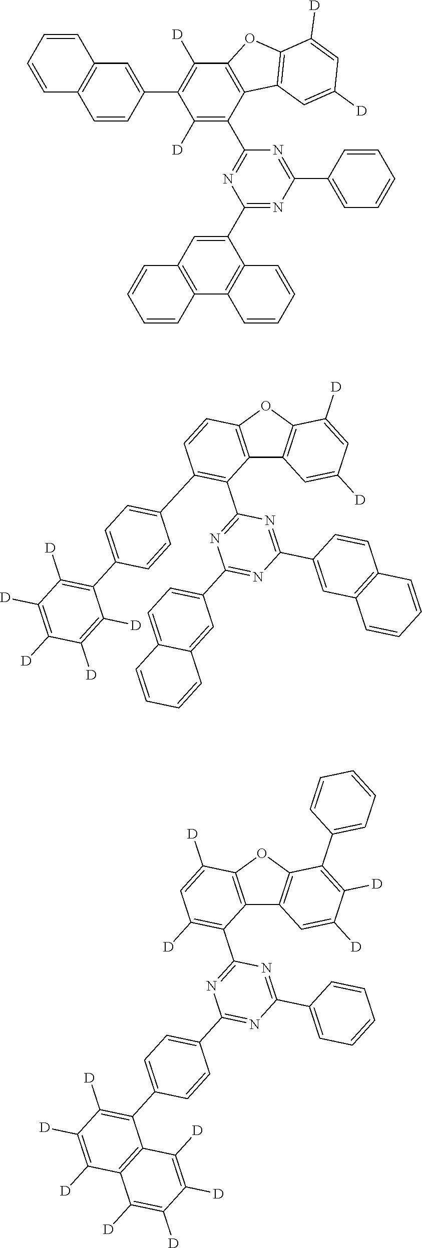 [SMILES: [2H]c1cc([2H])c2oc3c([2H])c(-c4ccc5ccccc5c4)c([2H])c(-c4nc(-c5ccccc5)nc(-c5cc6ccccc6c6ccccc56)n4)c3c2c1 ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc2c(oc3c([2H])cc([2H])c(-c4nc(-c5ccccc5)nc(-c5ccc(-c6c([2H])c([2H])c([2H])c7c([2H])c([2H])c([2H])c([2H])c67)cc5)n4)c32)c(-c2ccccc2)c1[2H]]

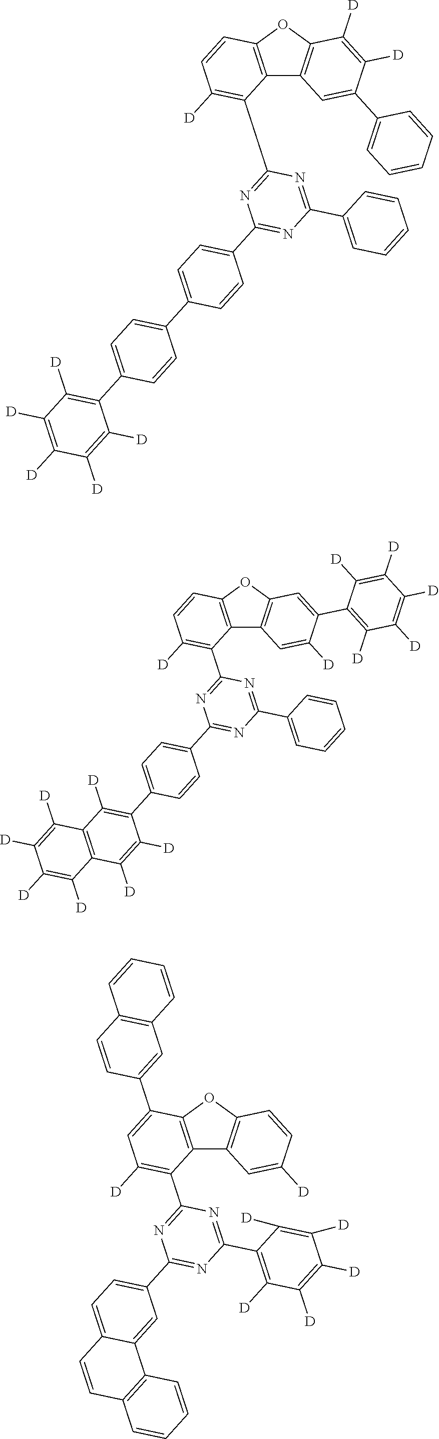 [SMILES: [2H]c1ccc2oc3c([2H])c([2H])c(-c4ccccc4)cc3c2c1-c1nc(-c2ccccc2)nc(-c2ccc(-c3ccc(-c4c([2H])c([2H])c([2H])c([2H])c4[2H])cc3)cc2)n1 ; [2H]c1cc2c(cc1-c1c([2H])c([2H])c([2H])c([2H])c1[2H])oc1ccc([2H])c(-c3nc(-c4ccccc4)nc(-c4ccc(-c5c([2H])c([2H])c6c([2H])c([2H])c([2H])c([2H])c6c5[2H])cc4)n3)c12 ; [2H]c1ccc2oc3c(-c4ccc5ccccc5c4)cc([2H])c(-c4nc(-c5ccc6ccc7ccccc7c6c5)nc(-c5c([2H])c([2H])c([2H])c([2H])c5[2H])n4)c3c2c1]

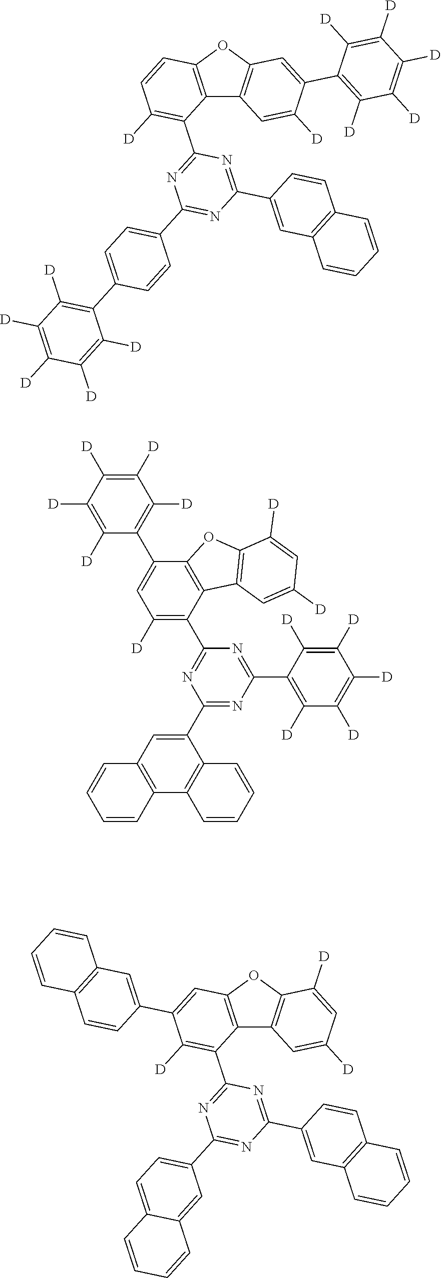 [SMILES: [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12 ; [2H]c1cc([2H])c2oc3c(-c4c([2H])c([2H])c([2H])c([2H])c4[2H])cc([2H])c(-c4nc(-c5c([2H])c([2H])c([2H])c([2H])c5[2H])nc(-c5cc6ccccc6c6ccccc56)n4)c3c2c1 ; [2H]c1cc([2H])c2oc3cc(-c4ccc5ccccc5c4)c([2H])c(-c4nc(-c5ccc6ccccc6c5)nc(-c5ccc6ccccc6c5)n4)c3c2c1]

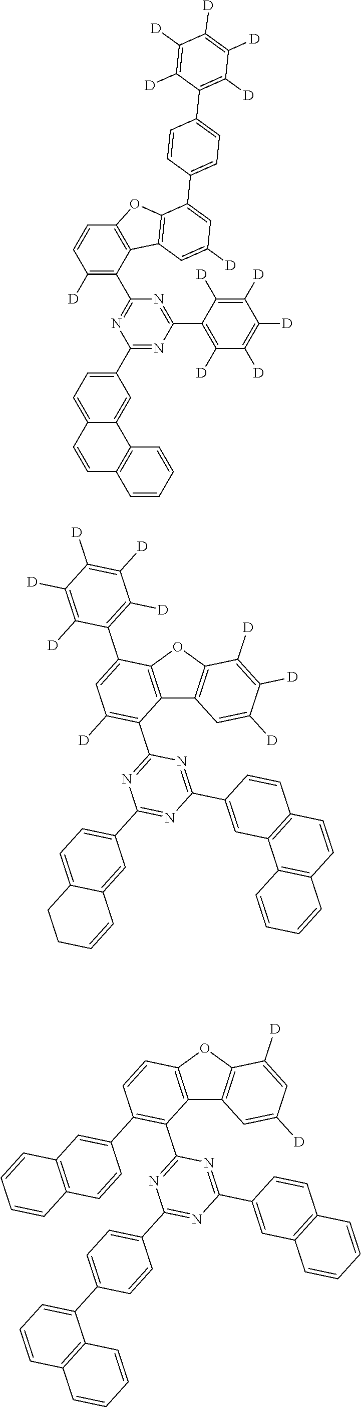 [SMILES: [2H]c1cc(-c2ccc(-c3c([2H])c([2H])c([2H])c([2H])c3[2H])cc2)c2oc3ccc([2H])c(-c4nc(-c5ccc6ccc7ccccc7c6c5)nc(-c5c([2H])c([2H])c([2H])c([2H])c5[2H])n4)c3c2c1 ; [2H]c1cc2c(oc3c(-c4c([2H])c([2H])c([2H])c([2H])c4[2H])cc([2H])c(-c4nc(-c5ccc6c(c5)C=CCC6)nc(-c5ccc6ccc7ccccc7c6c5)n4)c32)c([2H])c1[2H] ; [2H]c1cc([2H])c2oc3ccc(-c4ccc5ccccc5c4)c(-c4nc(-c5ccc(-c6cccc7ccccc67)cc5)nc(-c5ccc6ccccc6c5)n4)c3c2c1]

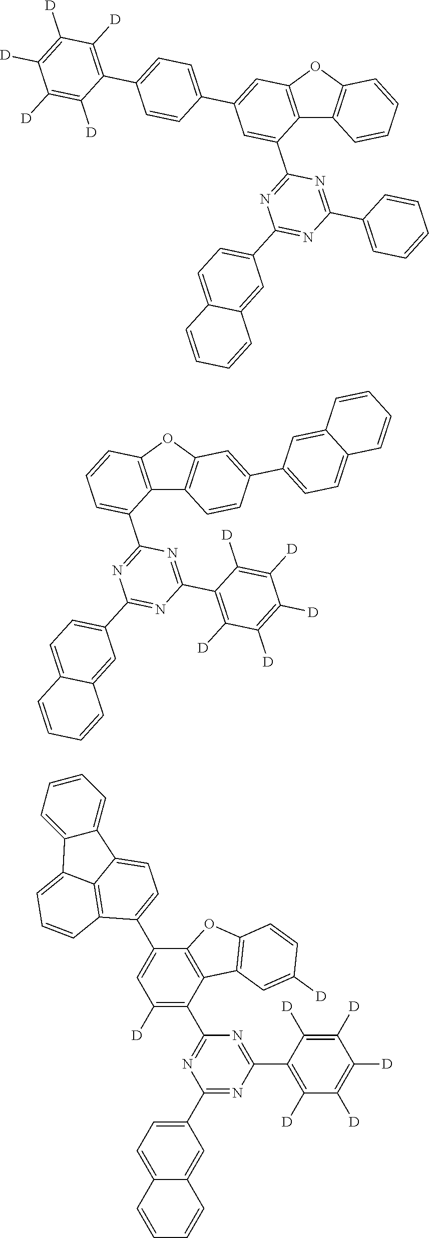

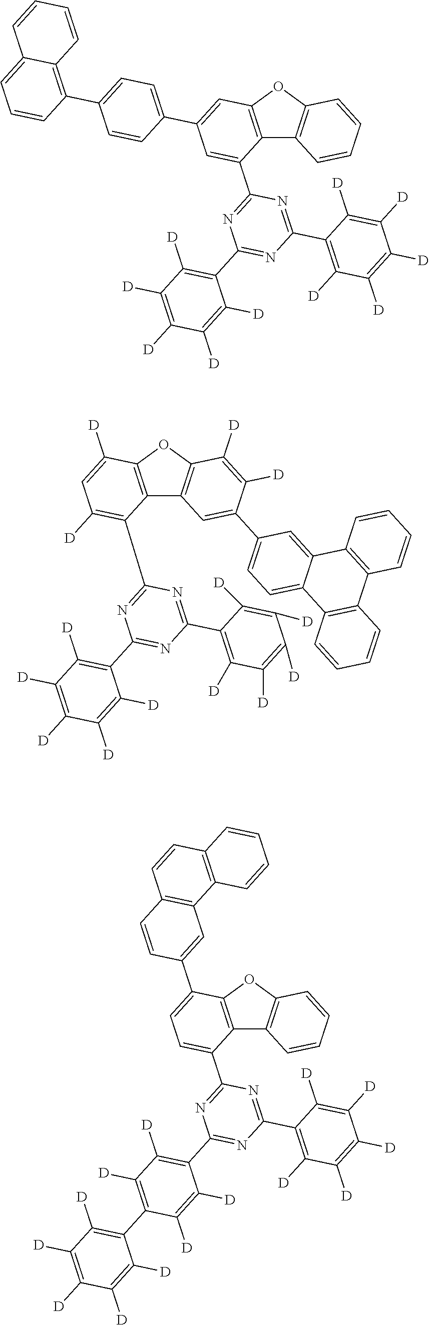 [SMILES: [2H]c1c([2H])c([2H])c(-c2nc(-c3c([2H])c([2H])c([2H])c([2H])c3[2H])nc(-c3cc(-c4ccc(-c5cccc6ccccc56)cc4)cc4oc5ccccc5c34)n2)c([2H])c1[2H] ; [2H]c1cc([2H])c2oc3c([2H])c([2H])c(-c4ccc5c6ccccc6c6ccccc6c5c4)cc3c2c1-c1nc(-c2c([2H])c([2H])c([2H])c([2H])c2[2H])nc(-c2c([2H])c([2H])c([2H])c([2H])c2[2H])n1 ; [2H]c1c([2H])c([2H])c(-c2nc(-c3c([2H])c([2H])c(-c4c([2H])c([2H])c([2H])c([2H])c4[2H])c([2H])c3[2H])nc(-c3ccc(-c4ccc5ccc6ccccc6c5c4)c4oc5ccccc5c34)n2)c([2H])c1[2H]]

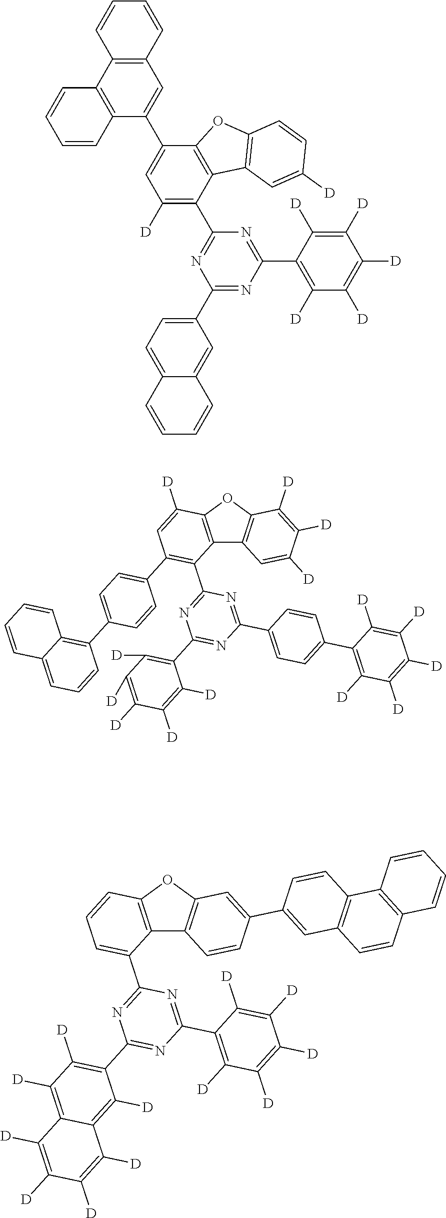 [SMILES: [2H]c1ccc2oc3c(-c4cc5ccccc5c5ccccc45)cc([2H])c(-c4nc(-c5ccc6ccccc6c5)nc(-c5c([2H])c([2H])c([2H])c([2H])c5[2H])n4)c3c2c1 ; [2H]c1cc2c(oc3c([2H])cc(-c4ccc(-c5cccc6ccccc56)cc4)c(-c4nc(-c5ccc(-c6c([2H])c([2H])c([2H])c([2H])c6[2H])cc5)nc(-c5c([2H])c([2H])c([2H])c([2H])c5[2H])n4)c32)c([2H])c1[2H] ; [2H]c1c([2H])c([2H])c(-c2nc(-c3c([2H])c([2H])c4c([2H])c([2H])c([2H])c([2H])c4c3[2H])nc(-c3cccc4oc5cc(-c6ccc7c(ccc8ccccc87)c6)ccc5c34)n2)c([2H])c1[2H]]

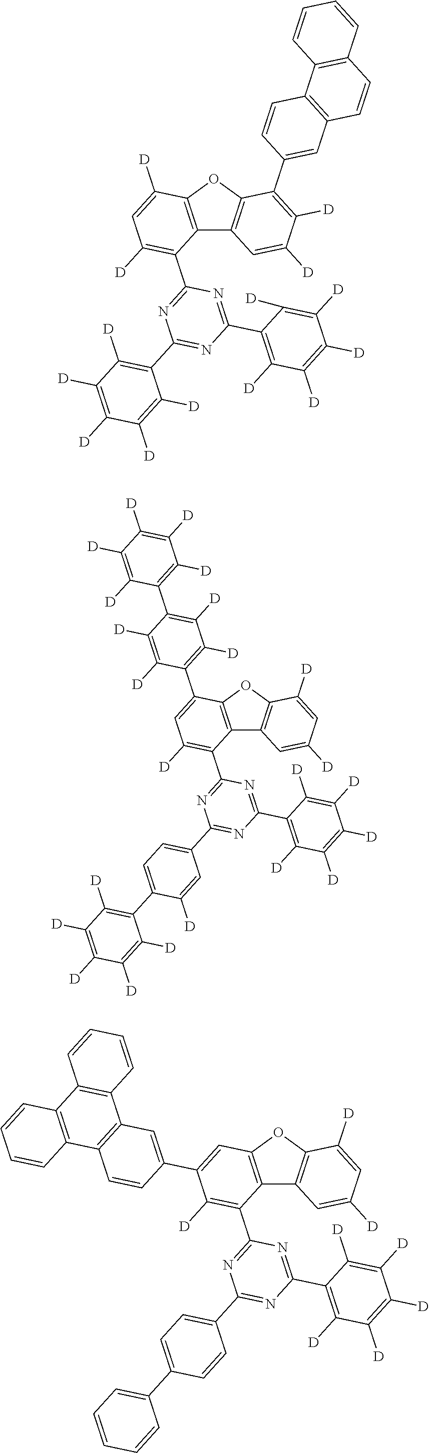

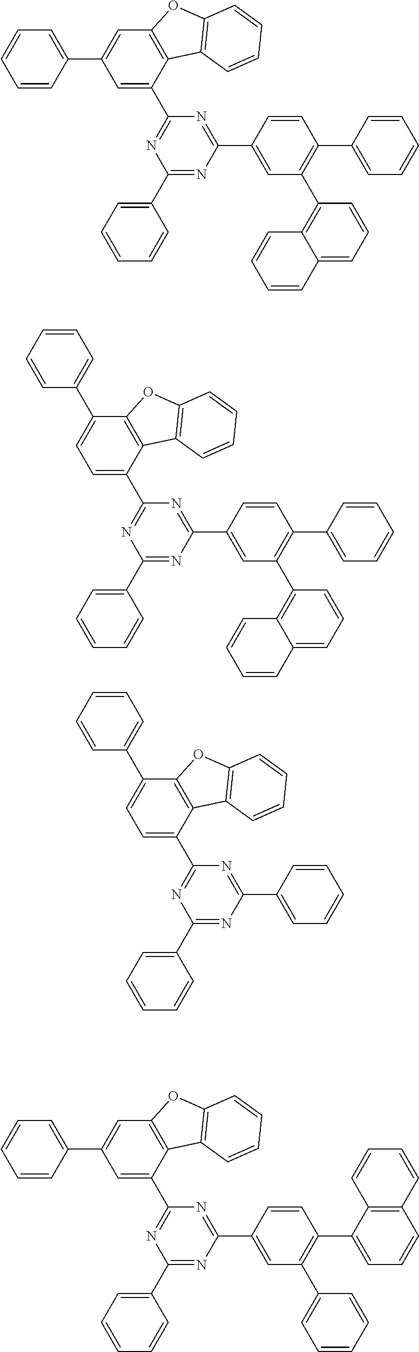

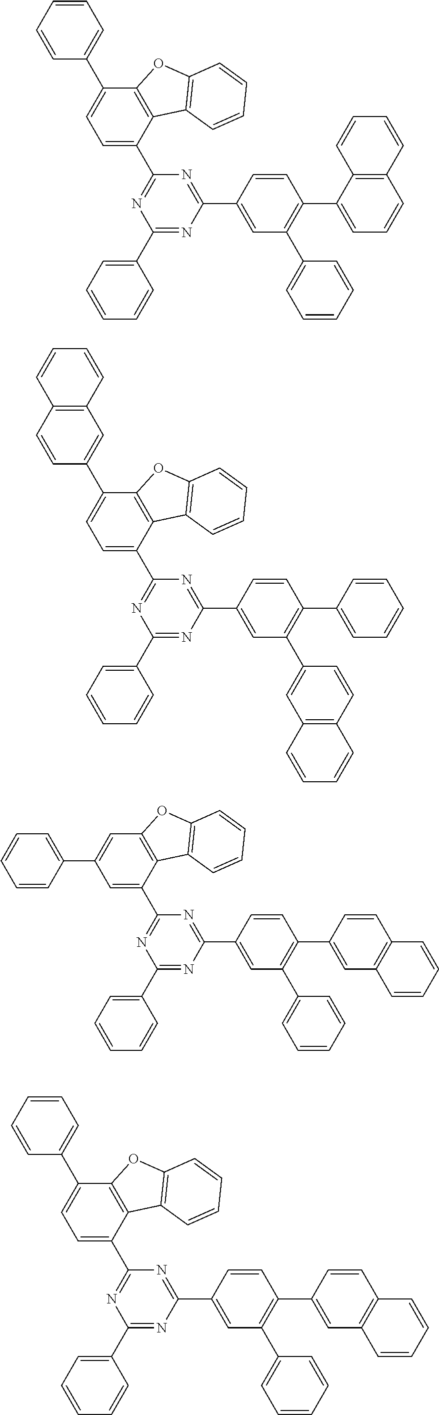

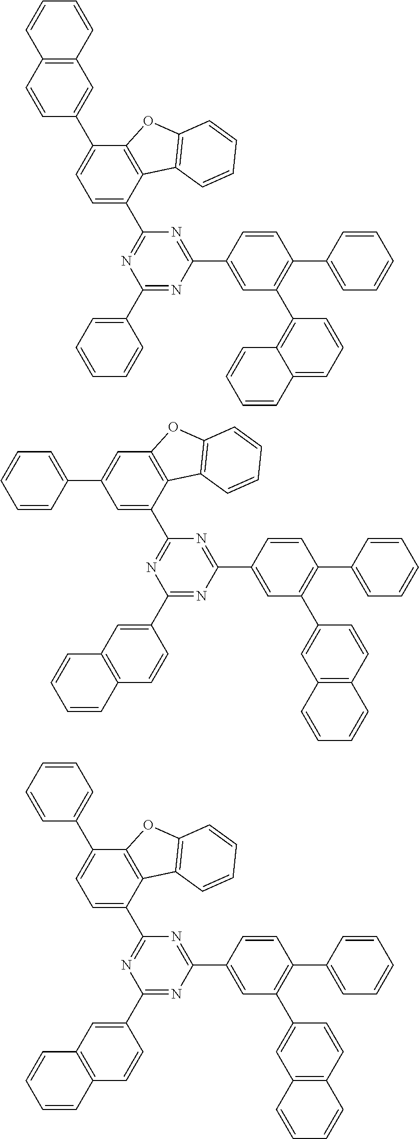

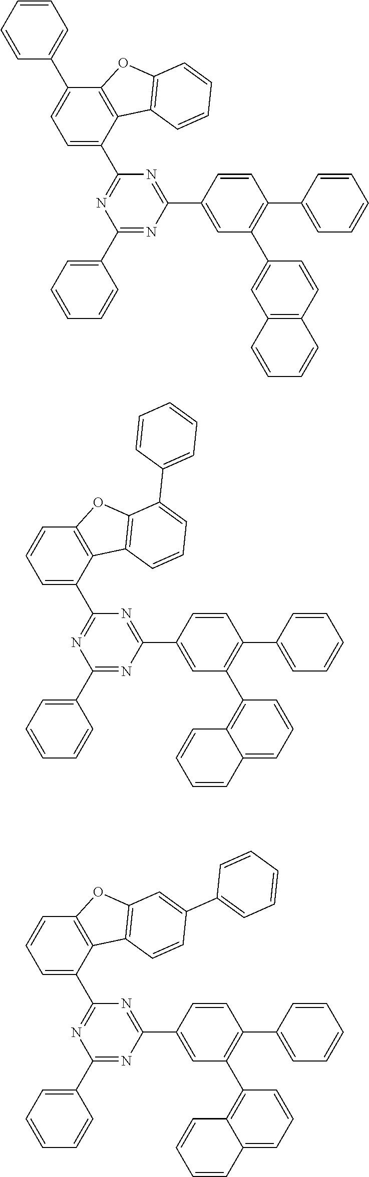 [SMILES: c1ccc(-c2nc(-c3ccc(-c4ccccc4)c(-c4ccc5ccccc5c4)c3)nc(-c3ccc(-c4ccccc4)c4oc5ccccc5c34)n2)cc1 ; c1ccc(-c2nc(-c3ccc(-c4ccccc4)c(-c4cccc5ccccc45)c3)nc(-c3cccc4oc5c(-c6ccccc6)cccc5c34)n2)cc1 ; c1ccc(-c2ccc3c(c2)oc2cccc(-c4nc(-c5ccccc5)nc(-c5ccc(-c6ccccc6)c(-c6cccc7ccccc67)c5)n4)c23)cc1]

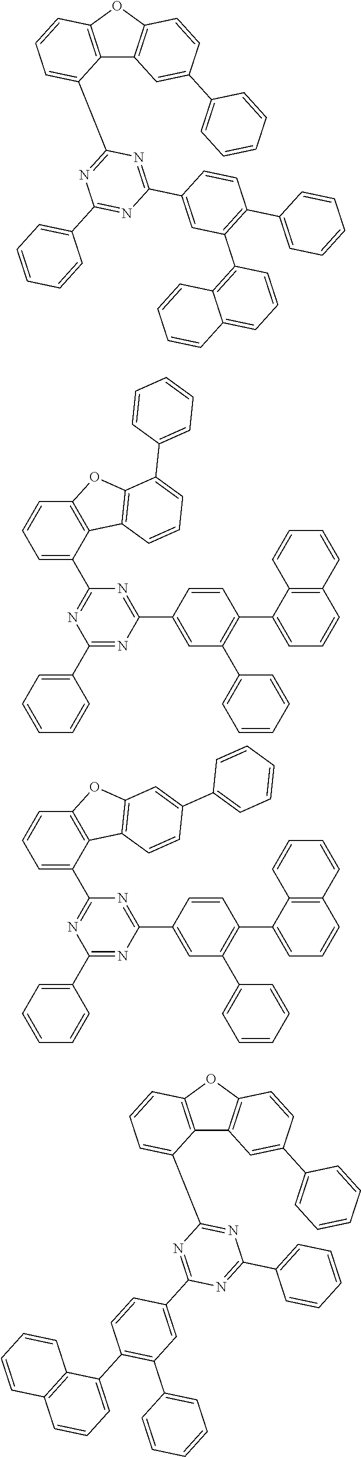

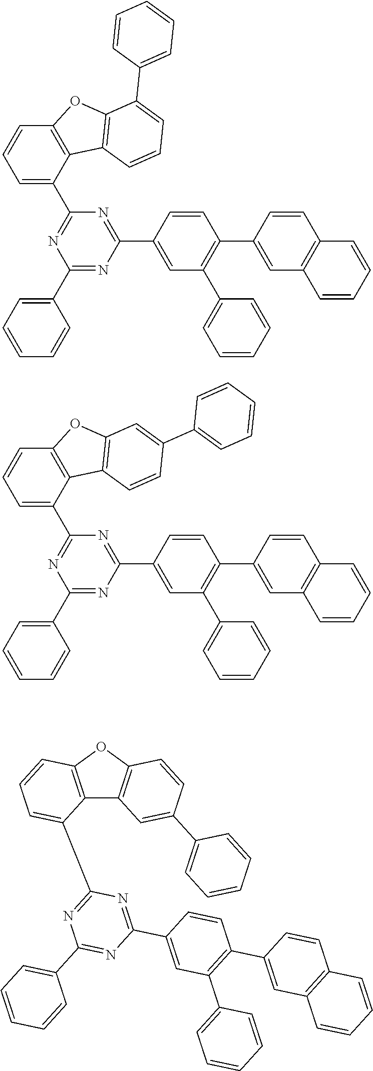

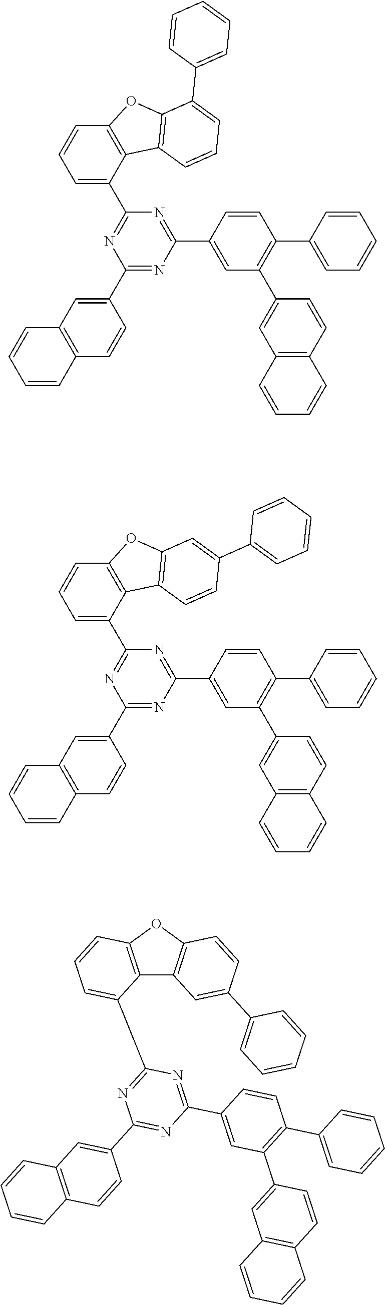 [SMILES: c1ccc(-c2ccc(-c3nc(-c4ccc5ccccc5c4)nc(-c4cccc5oc6c(-c7ccccc7)cccc6c45)n3)cc2-c2ccc3ccccc3c2)cc1 ; c1ccc(-c2ccc3c(c2)oc2cccc(-c4nc(-c5ccc(-c6ccccc6)c(-c6ccc7ccccc7c6)c5)nc(-c5ccc6ccccc6c5)n4)c23)cc1 ; c1ccc(-c2ccc3oc4cccc(-c5nc(-c6ccc(-c7ccccc7)c(-c7ccc8ccccc8c7)c6)nc(-c6ccc7ccccc7c6)n5)c4c3c2)cc1]

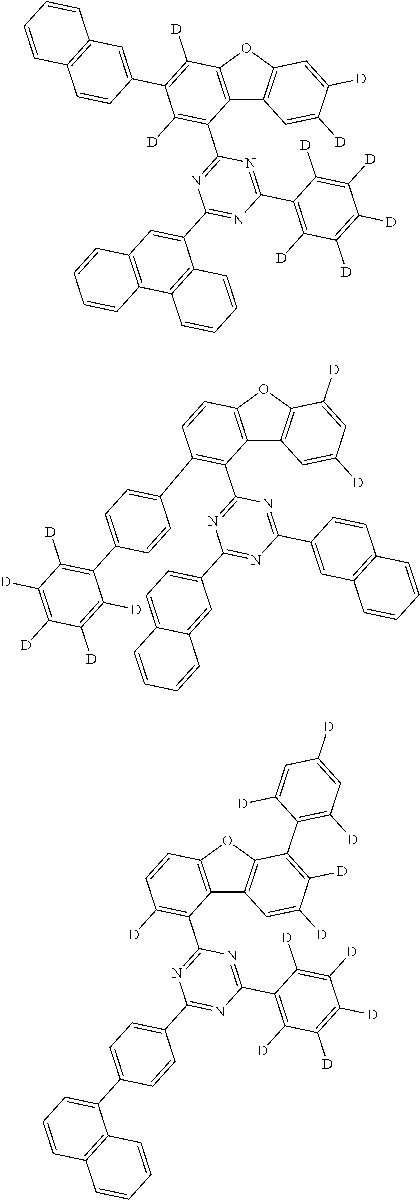 [SMILES: [2H]c1cc2oc3c([2H])c(-c4ccc5ccccc5c4)c([2H])c(-c4nc(-c5c([2H])c([2H])c([2H])c([2H])c5[2H])nc(-c5cc6ccccc6c6ccccc56)n4)c3c2cc1[2H] ; [2H]c1cc([2H])c2oc3ccc(-c4ccc(-c5c([2H])c([2H])c([2H])c([2H])c5[2H])cc4)c(-c4nc(-c5ccc6ccccc6c5)nc(-c5ccc6ccccc6c5)n4)c3c2c1 ; [2H]c1cc([2H])c(-c2c([2H])c([2H])cc3c2oc2ccc([2H])c(-c4nc(-c5ccc(-c6cccc7ccccc67)cc5)nc(-c5c([2H])c([2H])c([2H])c([2H])c5[2H])n4)c23)c([2H])c1]

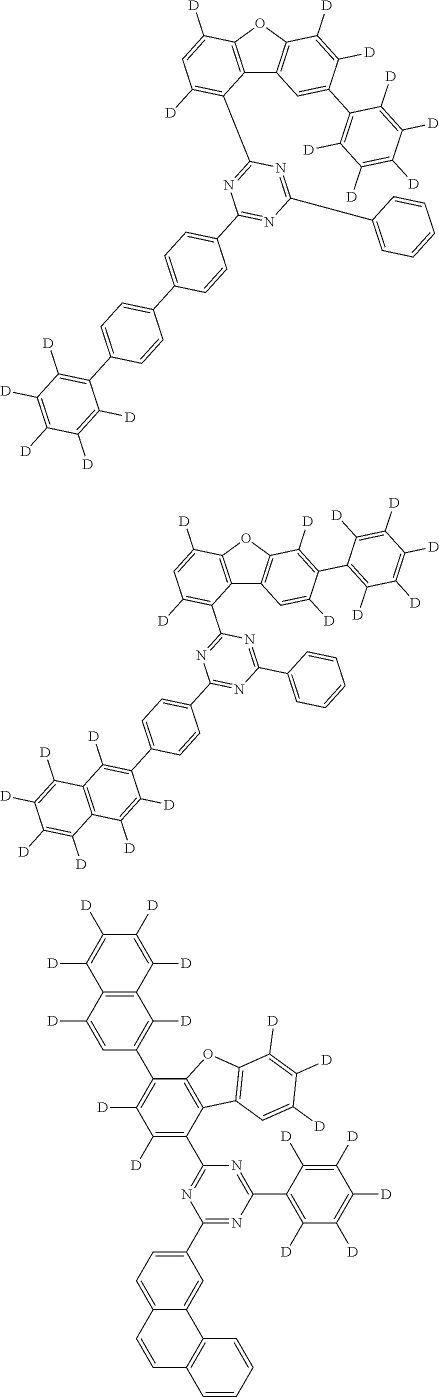

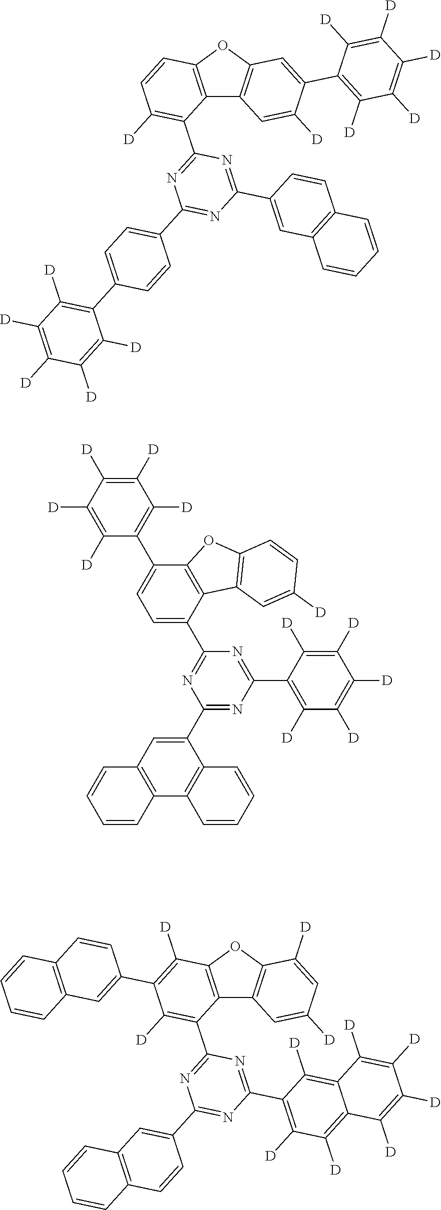 [SMILES: [2H]c1cc2c(cc1-c1c([2H])c([2H])c([2H])c([2H])c1[2H])oc1ccc([2H])c(-c3nc(-c4ccc(-c5c([2H])c([2H])c([2H])c([2H])c5[2H])cc4)nc(-c4ccc5ccccc5c4)n3)c12 ; [2H]c1ccc2oc3c(-c4c([2H])c([2H])c([2H])c([2H])c4[2H])ccc(-c4nc(-c5c([2H])c([2H])c([2H])c([2H])c5[2H])nc(-c5cc6ccccc6c6ccccc56)n4)c3c2c1 ; [2H]c1cc([2H])c2oc3c([2H])c(-c4ccc5ccccc5c4)c([2H])c(-c4nc(-c5ccc6ccccc6c5)nc(-c5c([2H])c([2H])c6c([2H])c([2H])c([2H])c([2H])c6c5[2H])n4)c3c2c1]

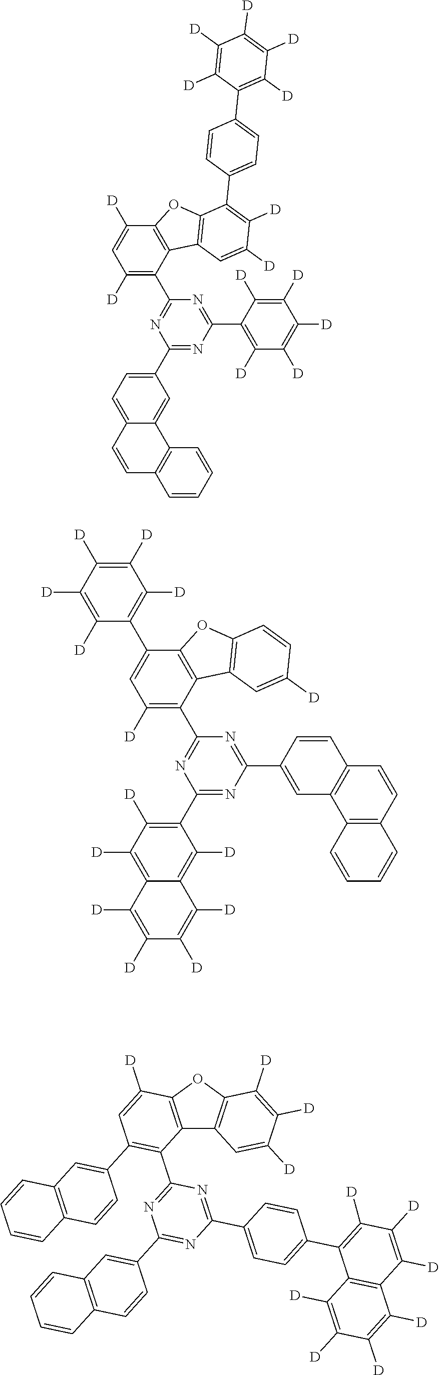

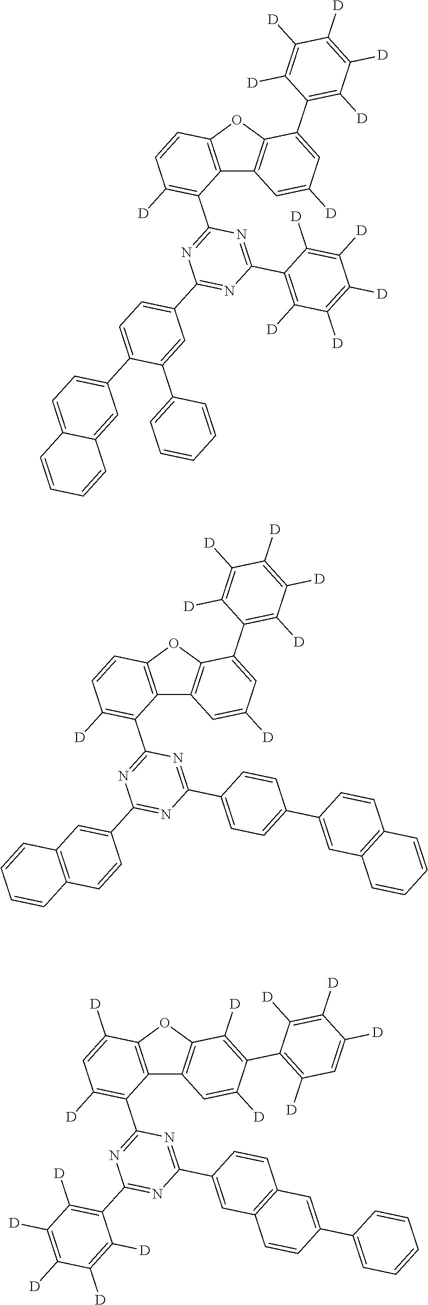 [SMILES: [2H]c1cc(-c2c([2H])c([2H])c([2H])c([2H])c2[2H])c2oc3ccc([2H])c(-c4nc(-c5ccc(-c6ccc7ccccc7c6)c(-c6ccccc6)c5)nc(-c5c([2H])c([2H])c([2H])c([2H])c5[2H])n4)c3c2c1 ; [2H]c1cc(-c2c([2H])c([2H])c([2H])c([2H])c2[2H])c2oc3ccc([2H])c(-c4nc(-c5ccc(-c6ccc7ccccc7c6)cc5)nc(-c5ccc6ccccc6c5)n4)c3c2c1 ; [2H]c1cc([2H])c(-c2c([2H])cc3c(oc4c([2H])cc([2H])c(-c5nc(-c6ccc7cc(-c8ccccc8)ccc7c6)nc(-c6c([2H])c([2H])c([2H])c([2H])c6[2H])n5)c43)c2[2H])c([2H])c1[2H]]

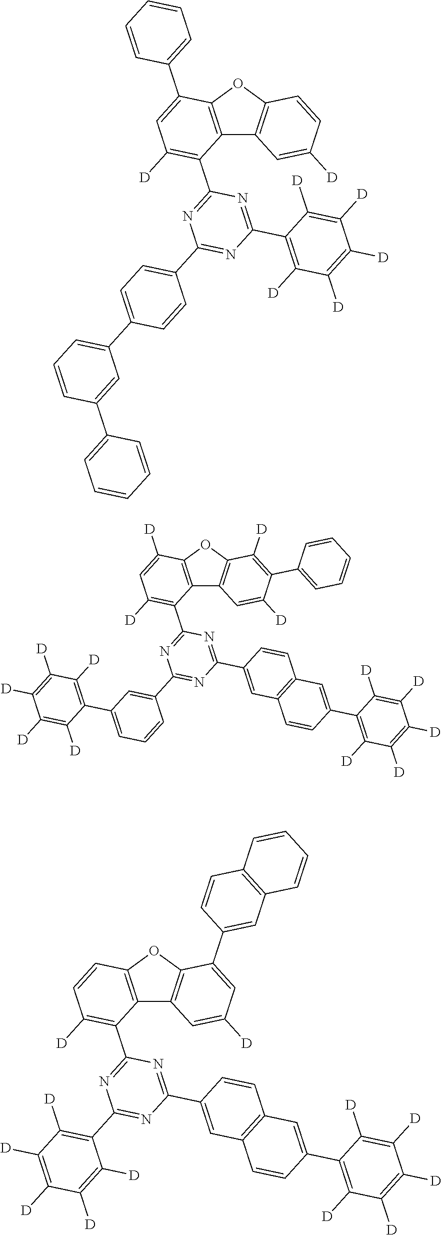

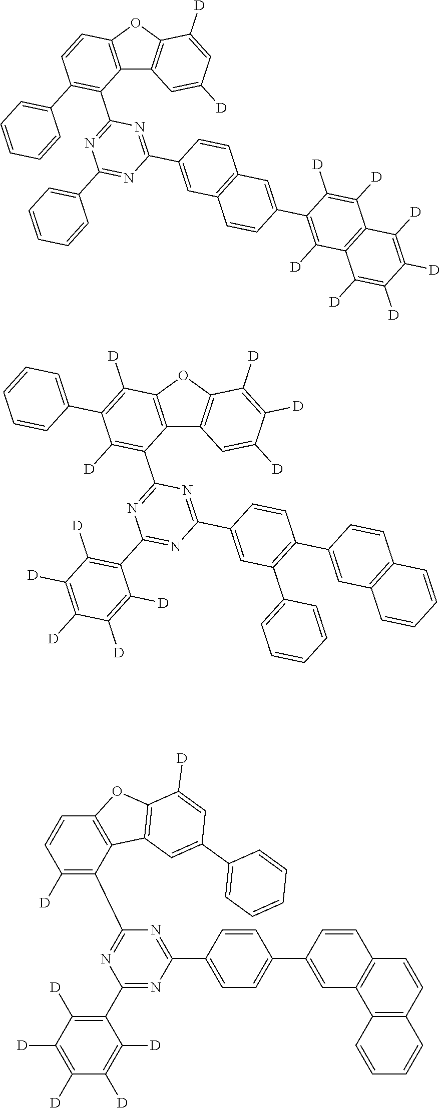 [SMILES: [2H]c1cc([2H])c2oc3ccc(-c4ccccc4)c(-c4nc(-c5ccccc5)nc(-c5ccc6cc(-c7c([2H])c([2H])c8c([2H])c([2H])c([2H])c([2H])c8c7[2H])ccc6c5)n4)c3c2c1 ; [2H]c1cc2c(oc3c([2H])c(-c4ccccc4)c([2H])c(-c4nc(-c5ccc(-c6ccc7ccccc7c6)c(-c6ccccc6)c5)nc(-c5c([2H])c([2H])c([2H])c([2H])c5[2H])n4)c32)c([2H])c1[2H] ; [2H]c1ccc2oc3c([2H])cc(-c4ccccc4)cc3c2c1-c1nc(-c2ccc(-c3ccc4ccc5ccccc5c4c3)cc2)nc(-c2c([2H])c([2H])c([2H])c([2H])c2[2H])n1]

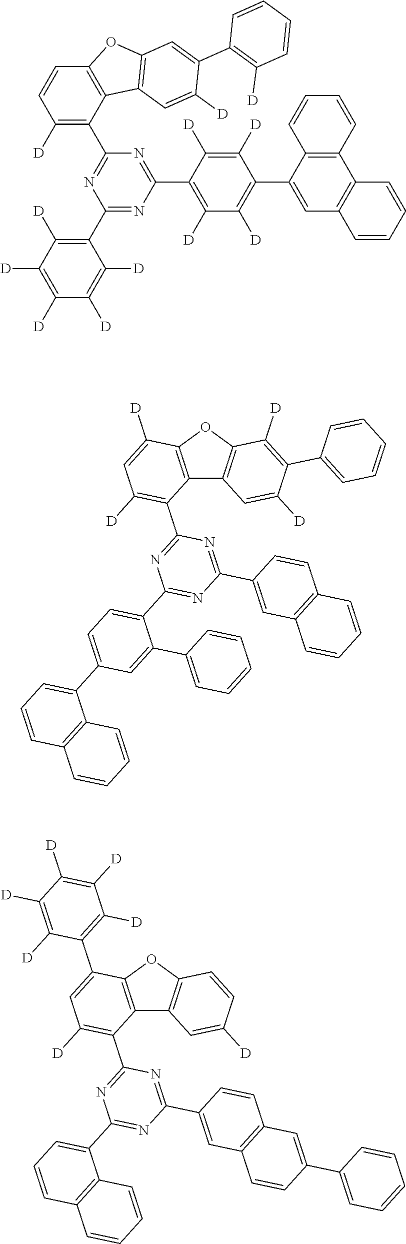 [SMILES: [2H]c1ccccc1-c1cc2oc3ccc([2H])c(-c4nc(-c5c([2H])c([2H])c([2H])c([2H])c5[2H])nc(-c5c([2H])c([2H])c(-c6cc7ccccc7c7ccccc67)c([2H])c5[2H])n4)c3c2cc1[2H] ; [2H]c1cc2c(oc3c([2H])cc([2H])c(-c4nc(-c5ccc6ccccc6c5)nc(-c5ccc(-c6cccc7ccccc67)cc5-c5ccccc5)n4)c32)c([2H])c1-c1ccccc1 ; [2H]c1ccc2oc3c(-c4c([2H])c([2H])c([2H])c([2H])c4[2H])cc([2H])c(-c4nc(-c5ccc6cc(-c7ccccc7)ccc6c5)nc(-c5cccc6ccccc56)n4)c3c2c1]

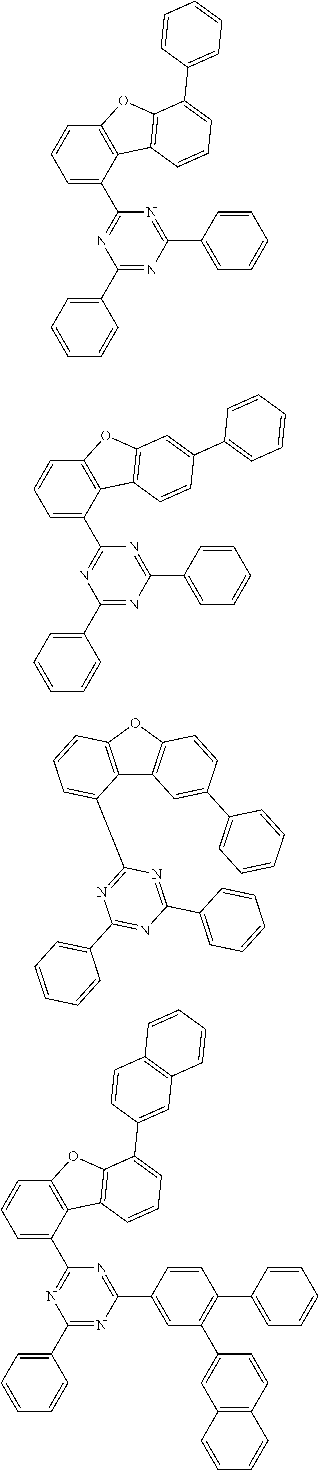

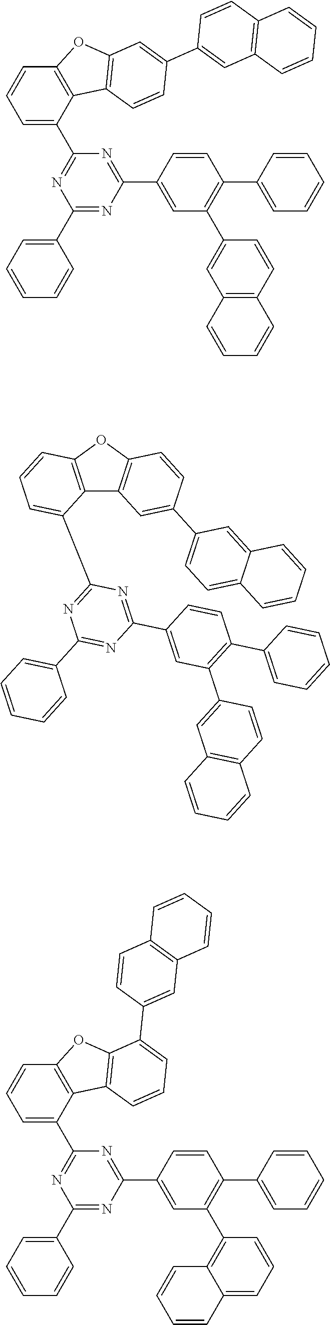 [SMILES: c1ccc(-c2nc(-c3ccc(-c4ccccc4)c(-c4ccc5ccccc5c4)c3)nc(-c3cccc4oc5cc(-c6ccc7ccccc7c6)ccc5c34)n2)cc1 ; c1ccc(-c2nc(-c3ccc(-c4ccccc4)c(-c4ccc5ccccc5c4)c3)nc(-c3cccc4oc5ccc(-c6ccc7ccccc7c6)cc5c34)n2)cc1 ; c1ccc(-c2nc(-c3ccc(-c4ccccc4)c(-c4cccc5ccccc45)c3)nc(-c3cccc4oc5c(-c6ccc7ccccc7c6)cccc5c34)n2)cc1]

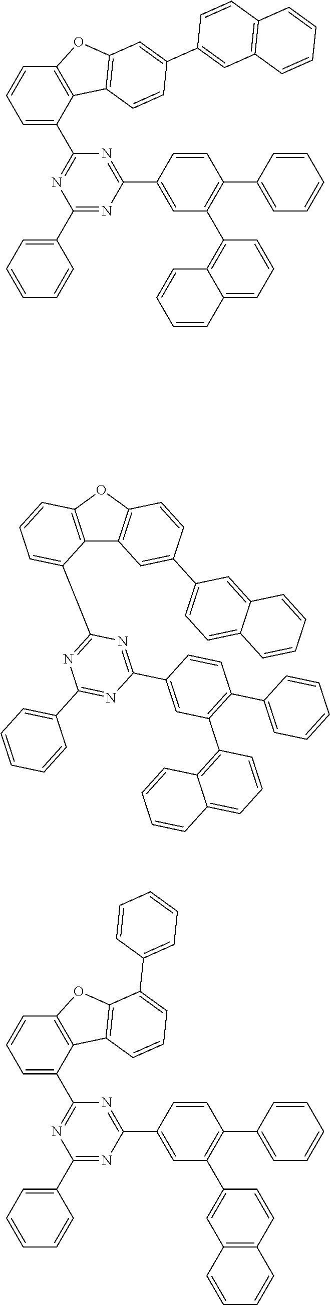 [SMILES: c1ccc(-c2nc(-c3ccc(-c4ccccc4)c(-c4cccc5ccccc45)c3)nc(-c3cccc4oc5cc(-c6ccc7ccccc7c6)ccc5c34)n2)cc1 ; c1ccc(-c2nc(-c3ccc(-c4ccccc4)c(-c4cccc5ccccc45)c3)nc(-c3cccc4oc5ccc(-c6ccc7ccccc7c6)cc5c34)n2)cc1 ; c1ccc(-c2nc(-c3ccc(-c4ccccc4)c(-c4ccc5ccccc5c4)c3)nc(-c3cccc4oc5c(-c6ccccc6)cccc5c34)n2)cc1]

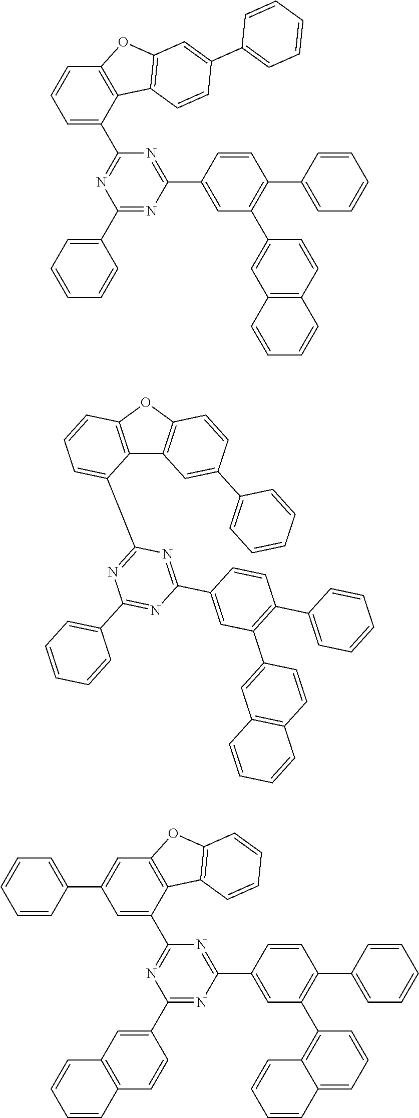 [SMILES: c1ccc(-c2ccc3c(c2)oc2cccc(-c4nc(-c5ccccc5)nc(-c5ccc(-c6ccccc6)c(-c6ccc7ccccc7c6)c5)n4)c23)cc1 ; c1ccc(-c2ccc3oc4cccc(-c5nc(-c6ccccc6)nc(-c6ccc(-c7ccccc7)c(-c7ccc8ccccc8c7)c6)n5)c4c3c2)cc1 ; c1ccc(-c2cc(-c3nc(-c4ccc(-c5ccccc5)c(-c5cccc6ccccc56)c4)nc(-c4ccc5ccccc5c4)n3)c3c(c2)oc2ccccc23)cc1]

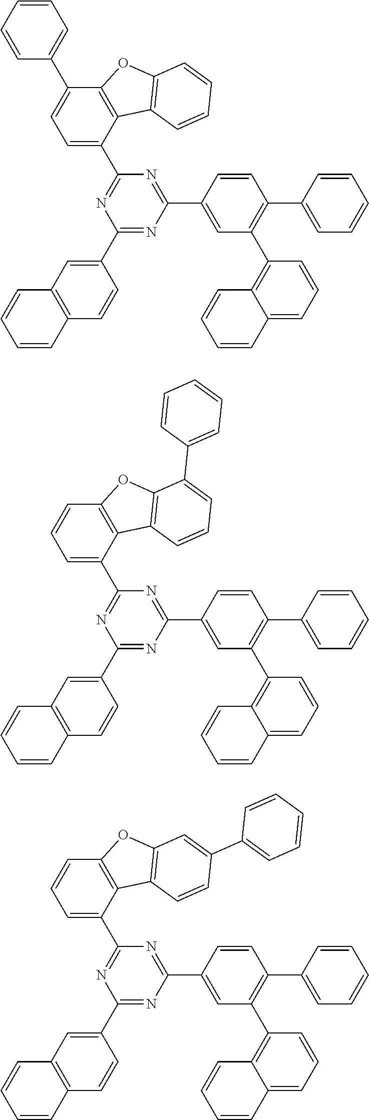

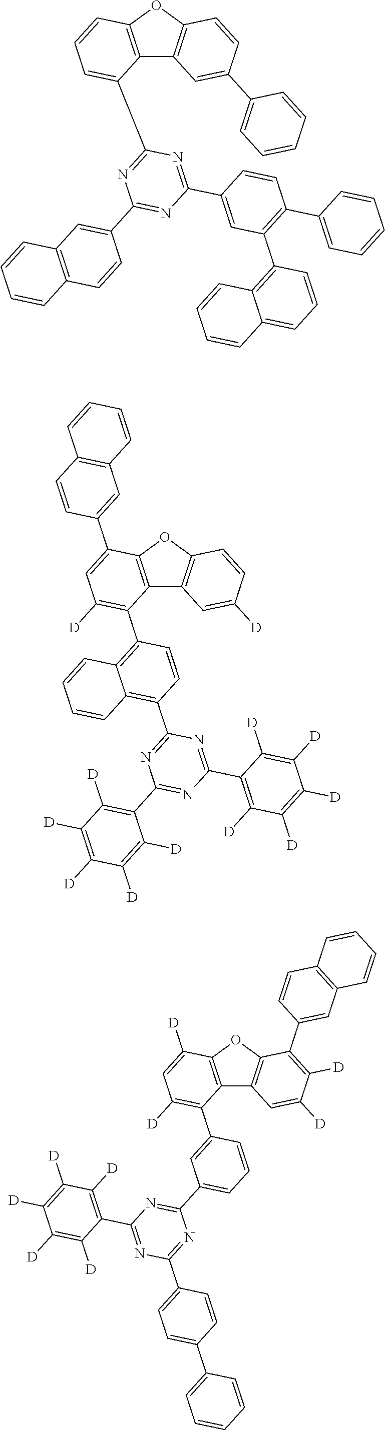 [SMILES: c1ccc(-c2ccc3oc4cccc(-c5nc(-c6ccc(-c7ccccc7)c(-c7cccc8ccccc78)c6)nc(-c6ccc7ccccc7c6)n5)c4c3c2)cc1 ; [2H]c1ccc2oc3c(-c4ccc5ccccc5c4)cc([2H])c(-c4ccc(-c5nc(-c6c([2H])c([2H])c([2H])c([2H])c6[2H])nc(-c6c([2H])c([2H])c([2H])c([2H])c6[2H])n5)c5ccccc45)c3c2c1 ; [2H]c1cc2c(oc3c([2H])cc([2H])c(-c4cccc(-c5nc(-c6ccc(-c7ccccc7)cc6)nc(-c6c([2H])c([2H])c([2H])c([2H])c6[2H])n5)c4)c32)c(-c2ccc3ccccc3c2)c1[2H]]

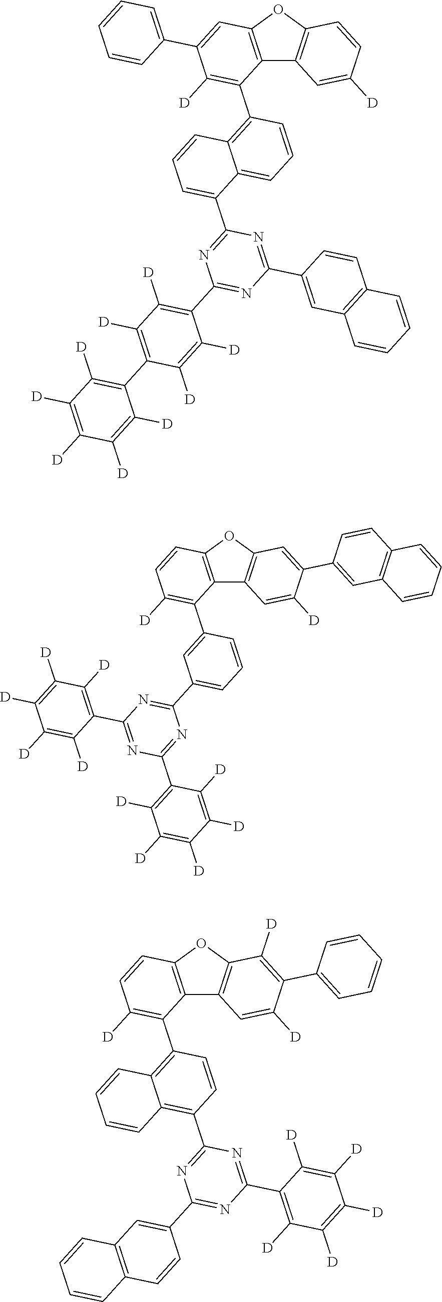 [SMILES: [2H]c1ccc2oc3cc(-c4ccccc4)c([2H])c(-c4cccc5c(-c6nc(-c7ccc8ccccc8c7)nc(-c7c([2H])c([2H])c(-c8c([2H])c([2H])c([2H])c([2H])c8[2H])c([2H])c7[2H])n6)cccc45)c3c2c1 ; [2H]c1cc2c(cc1-c1ccc3ccccc3c1)oc1ccc([2H])c(-c3cccc(-c4nc(-c5c([2H])c([2H])c([2H])c([2H])c5[2H])nc(-c5c([2H])c([2H])c([2H])c([2H])c5[2H])n4)c3)c12 ; [2H]c1cc2c(oc3ccc([2H])c(-c4ccc(-c5nc(-c6ccc7ccccc7c6)nc(-c6c([2H])c([2H])c([2H])c([2H])c6[2H])n5)c5ccccc45)c32)c([2H])c1-c1ccccc1]

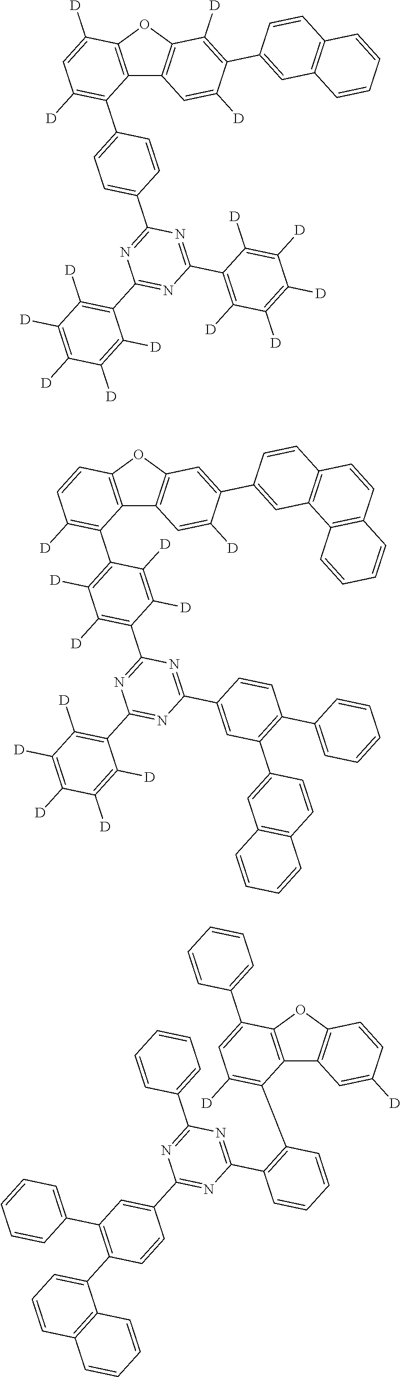 [SMILES: [2H]c1cc2c(oc3c([2H])cc([2H])c(-c4ccc(-c5nc(-c6c([2H])c([2H])c([2H])c([2H])c6[2H])nc(-c6c([2H])c([2H])c([2H])c([2H])c6[2H])n5)cc4)c32)c([2H])c1-c1ccc2ccccc2c1 ; [2H]c1cc2c(cc1-c1ccc3ccc4ccccc4c3c1)oc1ccc([2H])c(-c3c([2H])c([2H])c(-c4nc(-c5ccc(-c6ccccc6)c(-c6ccc7ccccc7c6)c5)nc(-c5c([2H])c([2H])c([2H])c([2H])c5[2H])n4)c([2H])c3[2H])c12 ; [2H]c1ccc2oc3c(-c4ccccc4)cc([2H])c(-c4ccccc4-c4nc(-c5ccccc5)nc(-c5ccc(-c6cccc7ccccc67)c(-c6ccccc6)c5)n4)c3c2c1]

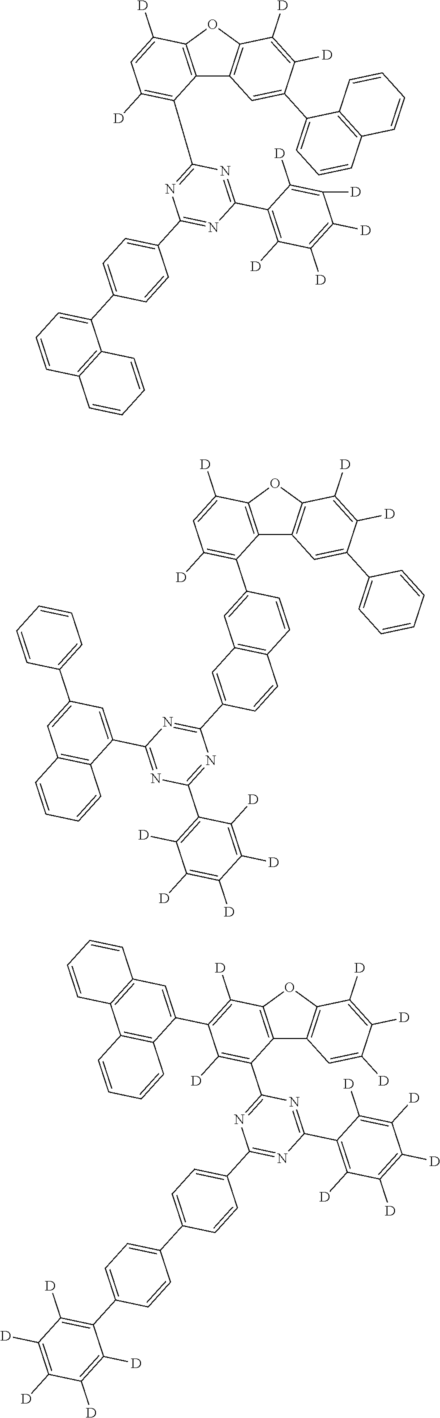 [SMILES: [2H]c1cc([2H])c2oc3c([2H])c([2H])c(-c4cccc5ccccc45)cc3c2c1-c1nc(-c2ccc(-c3cccc4ccccc34)cc2)nc(-c2c([2H])c([2H])c([2H])c([2H])c2[2H])n1 ; [2H]c1cc([2H])c2oc3c([2H])c([2H])c(-c4ccccc4)cc3c2c1-c1ccc2ccc(-c3nc(-c4c([2H])c([2H])c([2H])c([2H])c4[2H])nc(-c4cc(-c5ccccc5)cc5ccccc45)n3)cc2c1 ; [2H]c1cc2c(oc3c([2H])c(-c4cc5ccccc5c5ccccc45)c([2H])c(-c4nc(-c5ccc(-c6ccc(-c7c([2H])c([2H])c([2H])c([2H])c7[2H])cc6)cc5)nc(-c5c([2H])c([2H])c([2H])c([2H])c5[2H])n4)c32)c([2H])c1[2H]]

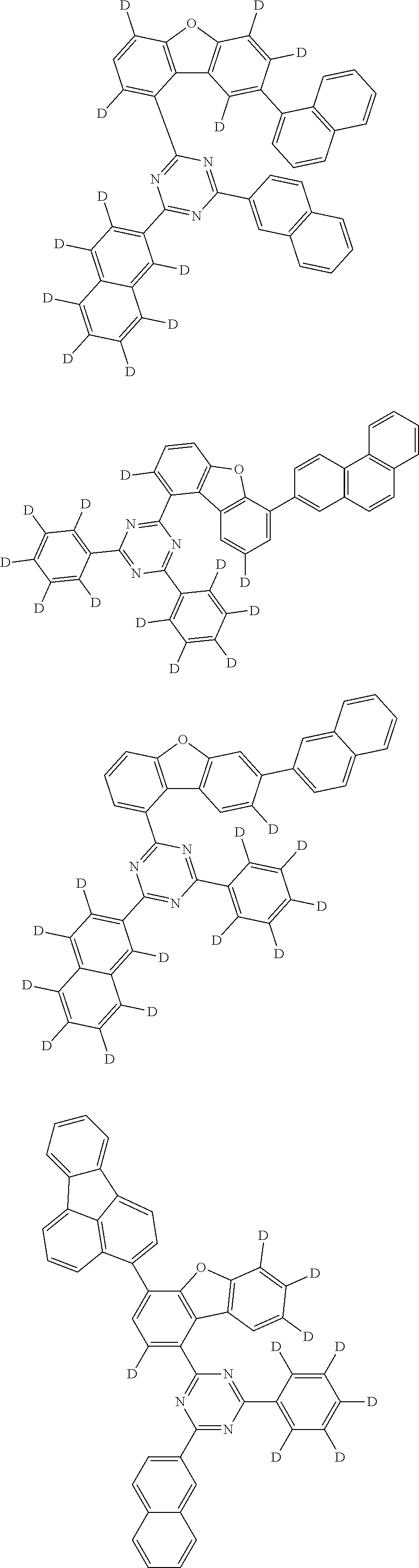

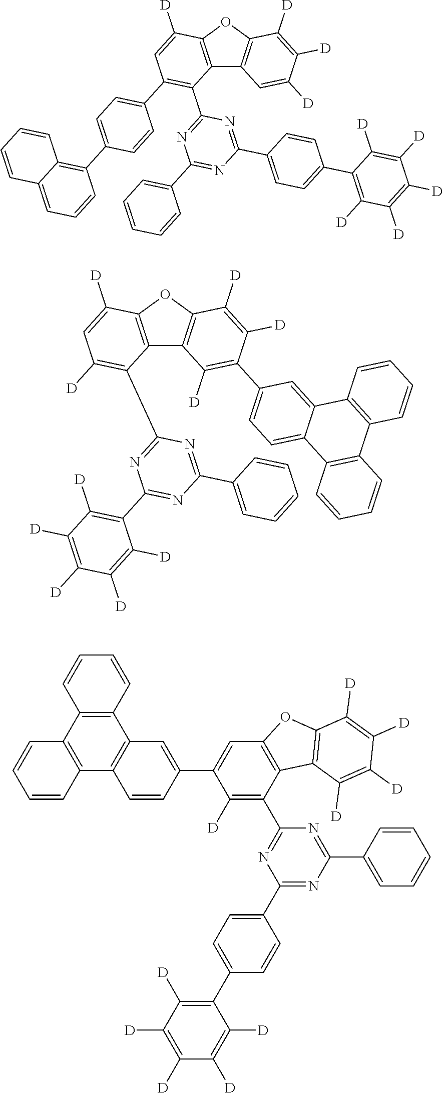 [SMILES: [2H]c1cc2c(oc3c([2H])cc(-c4ccc(-c5cccc6ccccc56)cc4)c(-c4nc(-c5ccccc5)nc(-c5ccc(-c6c([2H])c([2H])c([2H])c([2H])c6[2H])cc5)n4)c32)c([2H])c1[2H] ; [2H]c1cc([2H])c2oc3c([2H])c([2H])c(-c4ccc5c6ccccc6c6ccccc6c5c4)c([2H])c3c2c1-c1nc(-c2ccccc2)nc(-c2c([2H])c([2H])c([2H])c([2H])c2[2H])n1 ; [2H]c1c([2H])c([2H])c(-c2ccc(-c3nc(-c4ccccc4)nc(-c4c([2H])c(-c5ccc6c7ccccc7c7ccccc7c6c5)cc5oc6c([2H])c([2H])c([2H])c([2H])c6c45)n3)cc2)c([2H])c1[2H]]

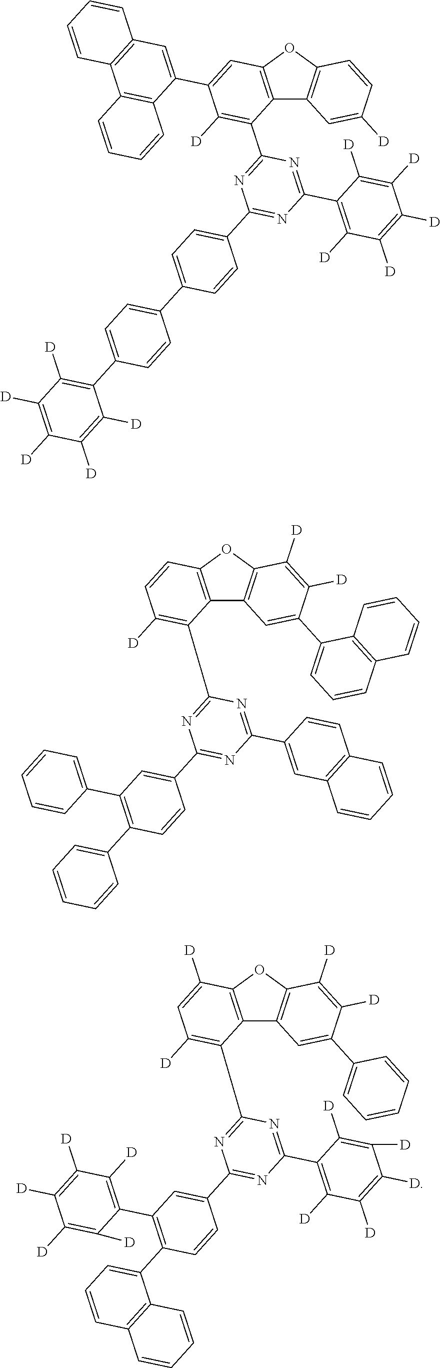

10. The organic light emitting device according to claim 1, wherein
the compound represented by Chemical Formula 2 is represented by one of the following Chemical Formulas 2-1 to 2-10: 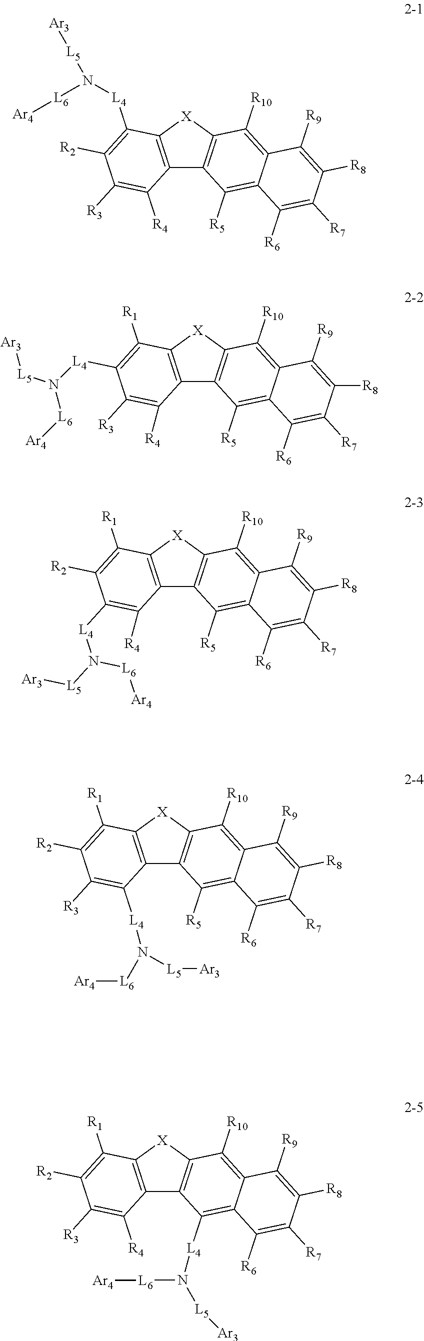 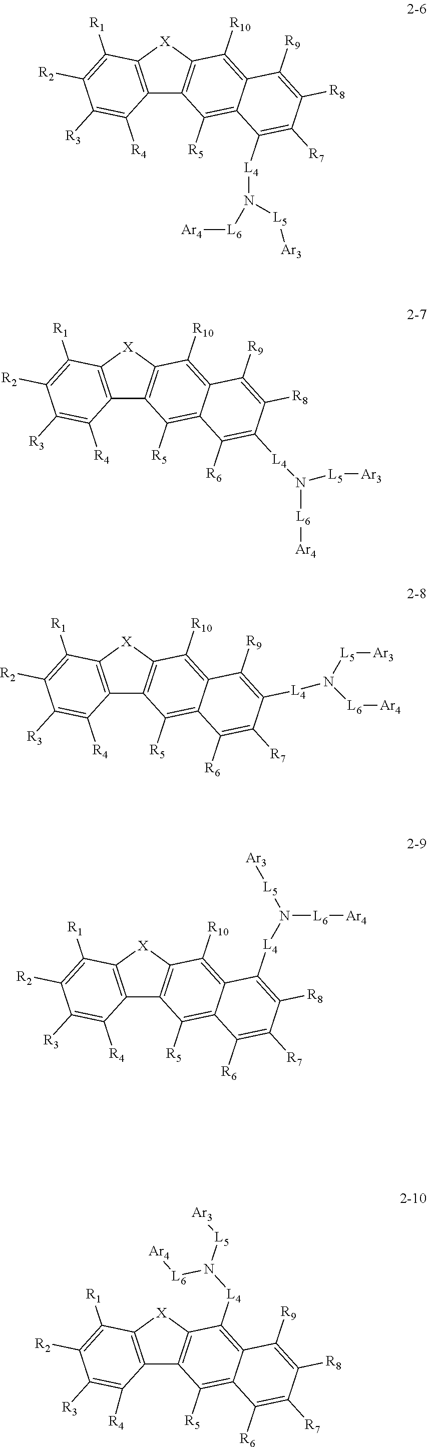
in Chemical Formulas 2-1 to 2-10,
X, R₁ to R₁₀, Ar₃, Ar₄, and L₄ to L₆ are the same as defined in claim 1.

11. The organic light emitting device according to claim 1, wherein
Ar₃ and Ar₄ are each independently phenyl, biphenyl, terphenyl, naphthyl, phenanthrenyl, triphenylenyl, (naphthyl)phenyl, (phenyl)naphthyl, fluorenyl, dimethylfluorenyl, diphenylfluorenyl, fluoranthenyl, dibenzofuranyl, dibenzothiophenyl, carbazolyl, or 9-phenylcarbazolyl,
each of which is unsubstituted or substituted with deuterium, phenyl, or triphenylsilyl.

12. The organic light emitting device according to claim 1, wherein
L₄ is a single bond, phenylene, biphenyldiyl, naphthalenediyl, or binaphthalenediyl,
each of which, except for a single bond, is unsubstituted or substituted with deuterium, phenyl, or naphthyl.

13. The organic light emitting device according to claim 1, wherein:
L₅ and L₆ are each independently a single bond, phenylene, biphenyldiyl, naphthalenediyl, or binaphthalenediyl,
each of which, except for a single bond, is unsubstituted or substituted with deuterium, phenyl, or naphthyl.

14. The organic light emitting device according to claim 1, wherein
the compound represented by Chemical Formula 2 is one selected from the following: 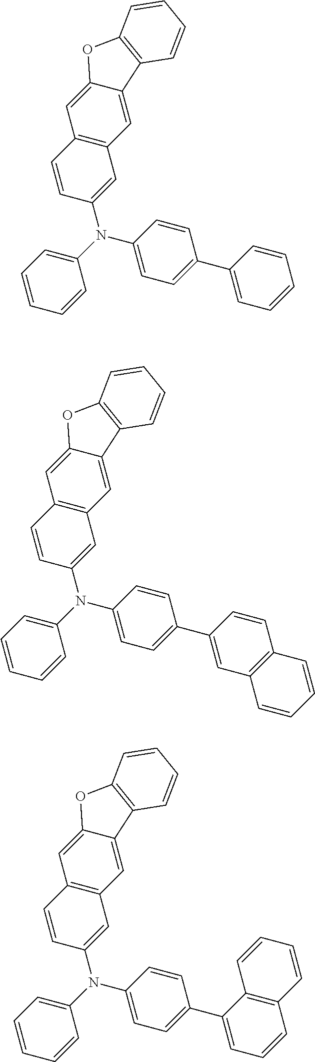 [SMILES: c1ccc(-c2ccc(N(c3ccccc3)c3ccc4cc5oc6ccccc6c5cc4c3)cc2)cc1 ; c1ccc(N(c2ccc(-c3ccc4ccccc4c3)cc2)c2ccc3cc4oc5ccccc5c4cc3c2)cc1 ; c1ccc(N(c2ccc(-c3cccc4ccccc34)cc2)c2ccc3cc4oc5ccccc5c4cc3c2)cc1] 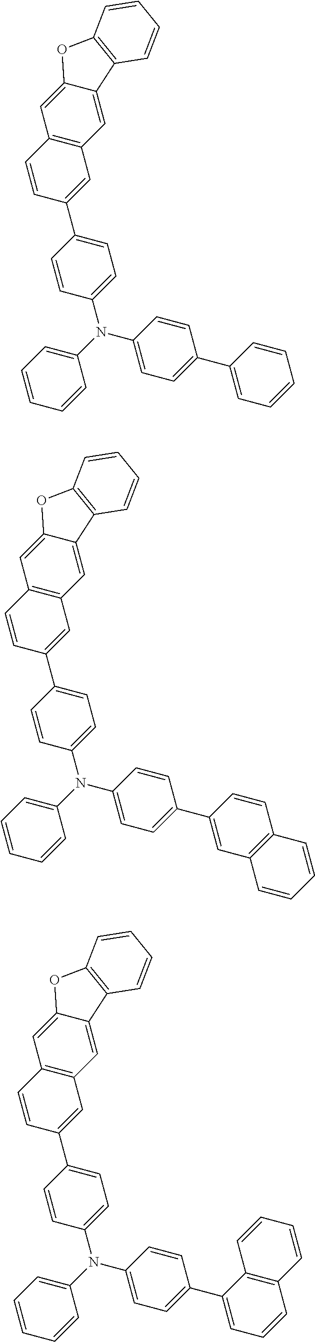 [SMILES: c1ccc(-c2ccc(N(c3ccccc3)c3ccc(-c4ccc5cc6oc7ccccc7c6cc5c4)cc3)cc2)cc1 ; c1ccc(N(c2ccc(-c3ccc4ccccc4c3)cc2)c2ccc(-c3ccc4cc5oc6ccccc6c5cc4c3)cc2)cc1 ; c1ccc(N(c2ccc(-c3ccc4cc5oc6ccccc6c5cc4c3)cc2)c2ccc(-c3cccc4ccccc34)cc2)cc1] 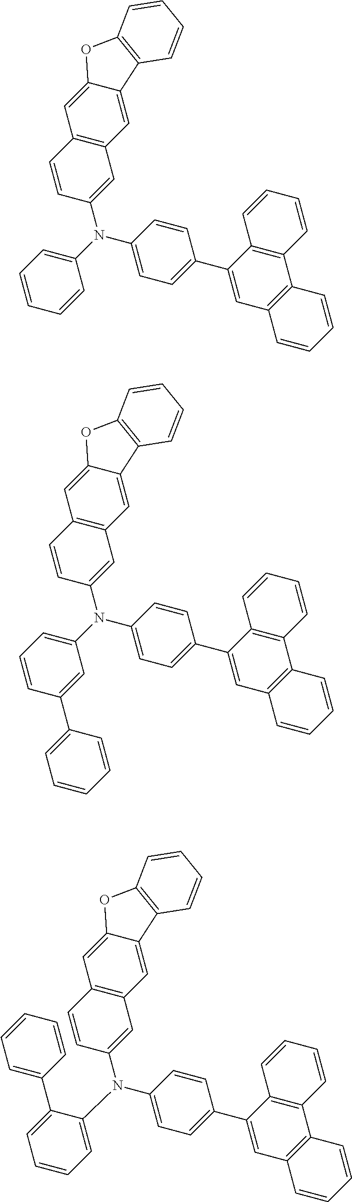 [SMILES: c1ccc(N(c2ccc(-c3cc4ccccc4c4ccccc34)cc2)c2ccc3cc4oc5ccccc5c4cc3c2)cc1 ; c1ccc(-c2cccc(N(c3ccc(-c4cc5ccccc5c5ccccc45)cc3)c3ccc4cc5oc6ccccc6c5cc4c3)c2)cc1 ; c1ccc(-c2ccccc2N(c2ccc(-c3cc4ccccc4c4ccccc34)cc2)c2ccc3cc4oc5ccccc5c4cc3c2)cc1] 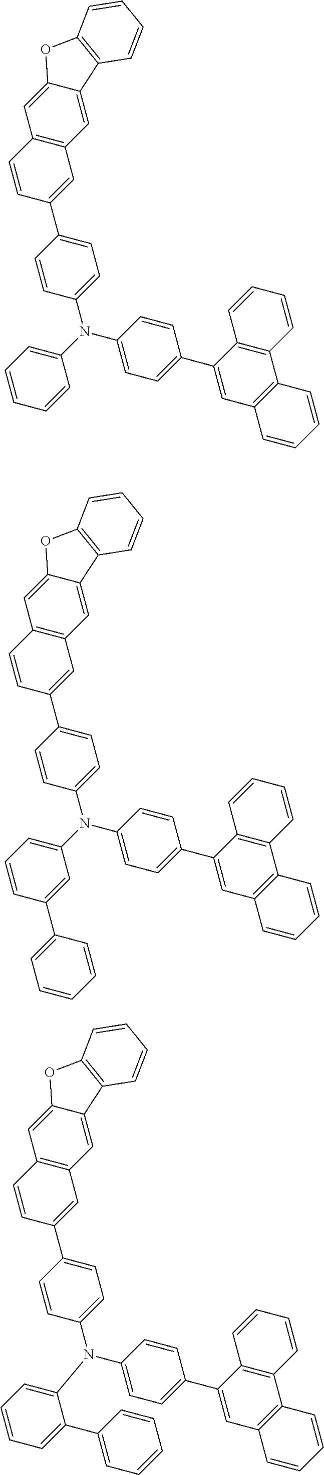 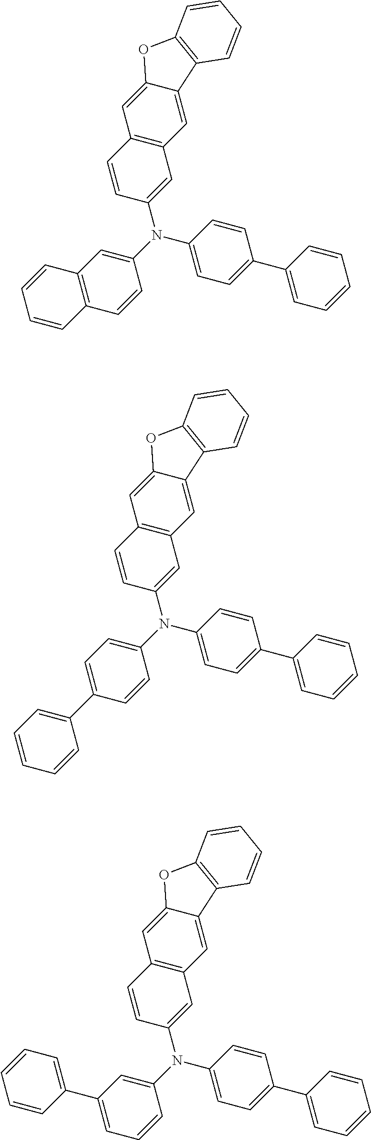 [SMILES: c1ccc(-c2ccc(N(c3ccc4ccccc4c3)c3ccc4cc5oc6ccccc6c5cc4c3)cc2)cc1 ; c1ccc(-c2ccc(N(c3ccc(-c4ccccc4)cc3)c3ccc4cc5oc6ccccc6c5cc4c3)cc2)cc1 ; c1ccc(-c2ccc(N(c3cccc(-c4ccccc4)c3)c3ccc4cc5oc6ccccc6c5cc4c3)cc2)cc1] 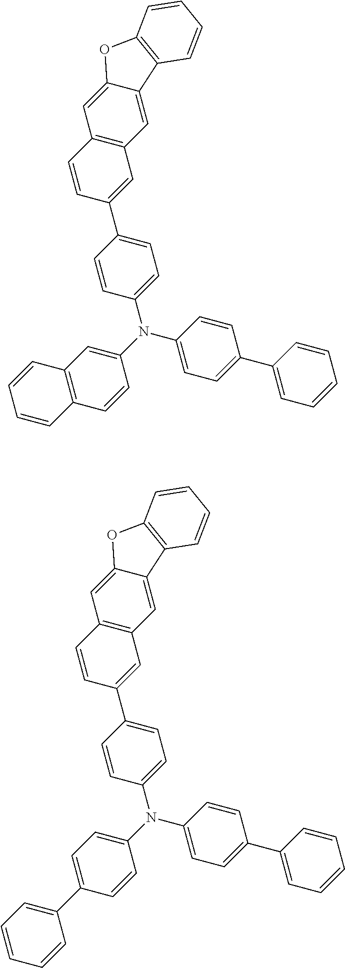 [SMILES: c1ccc(-c2ccc(N(c3ccc(-c4ccc5cc6oc7ccccc7c6cc5c4)cc3)c3ccc4ccccc4c3)cc2)cc1 ; c1ccc(-c2ccc(N(c3ccc(-c4ccccc4)cc3)c3ccc(-c4ccc5cc6oc7ccccc7c6cc5c4)cc3)cc2)cc1] 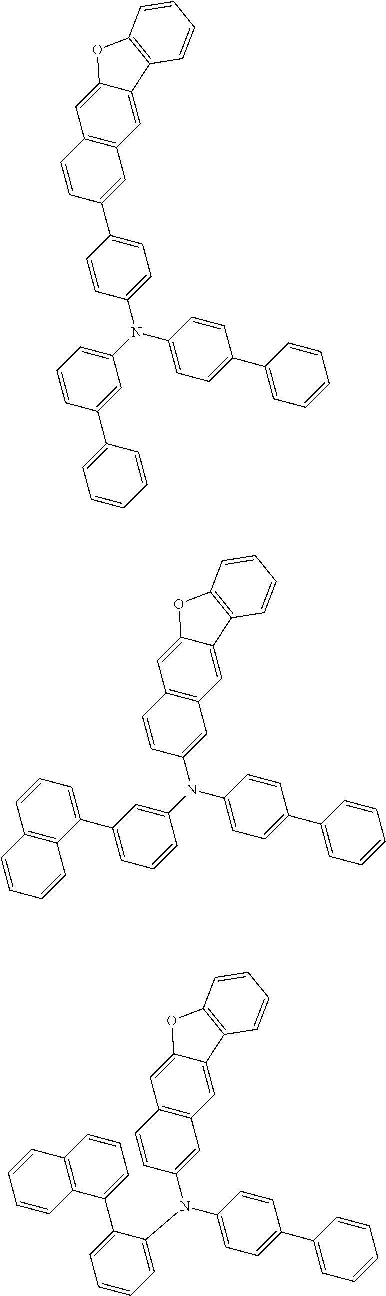 [SMILES: c1ccc(-c2ccc(N(c3ccc(-c4ccc5cc6oc7ccccc7c6cc5c4)cc3)c3cccc(-c4ccccc4)c3)cc2)cc1 ; c1ccc(-c2ccc(N(c3cccc(-c4cccc5ccccc45)c3)c3ccc4cc5oc6ccccc6c5cc4c3)cc2)cc1 ; c1ccc(-c2ccc(N(c3ccc4cc5oc6ccccc6c5cc4c3)c3ccccc3-c3cccc4ccccc34)cc2)cc1] 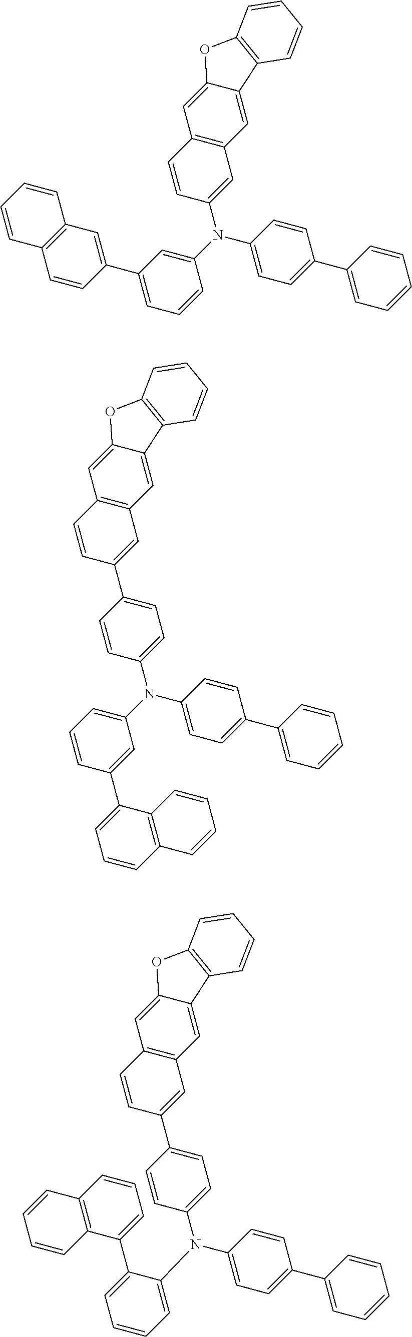 [SMILES: c1ccc(-c2ccc(N(c3cccc(-c4ccc5ccccc5c4)c3)c3ccc4cc5oc6ccccc6c5cc4c3)cc2)cc1 ; c1ccc(-c2ccc(N(c3ccc(-c4ccc5cc6oc7ccccc7c6cc5c4)cc3)c3cccc(-c4cccc5ccccc45)c3)cc2)cc1 ; c1ccc(-c2ccc(N(c3ccc(-c4ccc5cc6oc7ccccc7c6cc5c4)cc3)c3ccccc3-c3cccc4ccccc34)cc2)cc1] 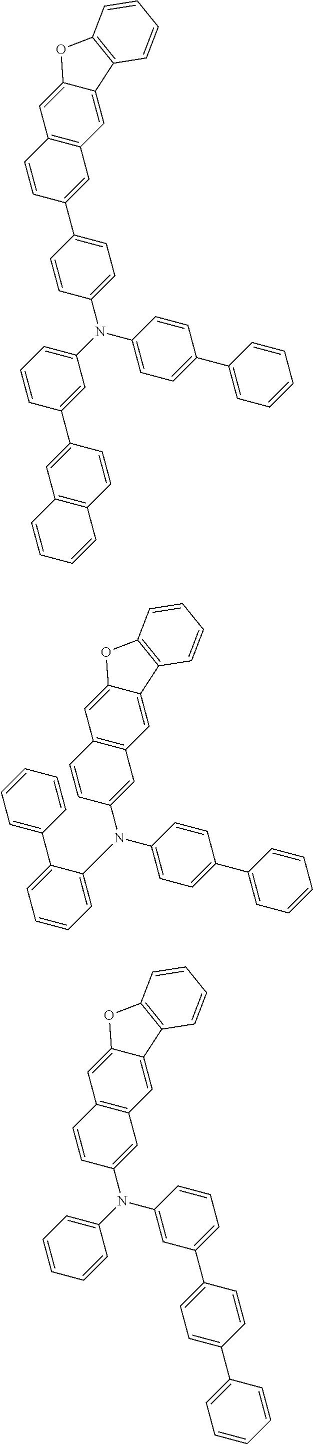 [SMILES: c1ccc(-c2ccc(N(c3ccc(-c4ccc5cc6oc7ccccc7c6cc5c4)cc3)c3cccc(-c4ccc5ccccc5c4)c3)cc2)cc1 ; c1ccc(-c2ccc(N(c3ccc4cc5oc6ccccc6c5cc4c3)c3ccccc3-c3ccccc3)cc2)cc1 ; c1ccc(-c2ccc(-c3cccc(N(c4ccccc4)c4ccc5cc6oc7ccccc7c6cc5c4)c3)cc2)cc1] 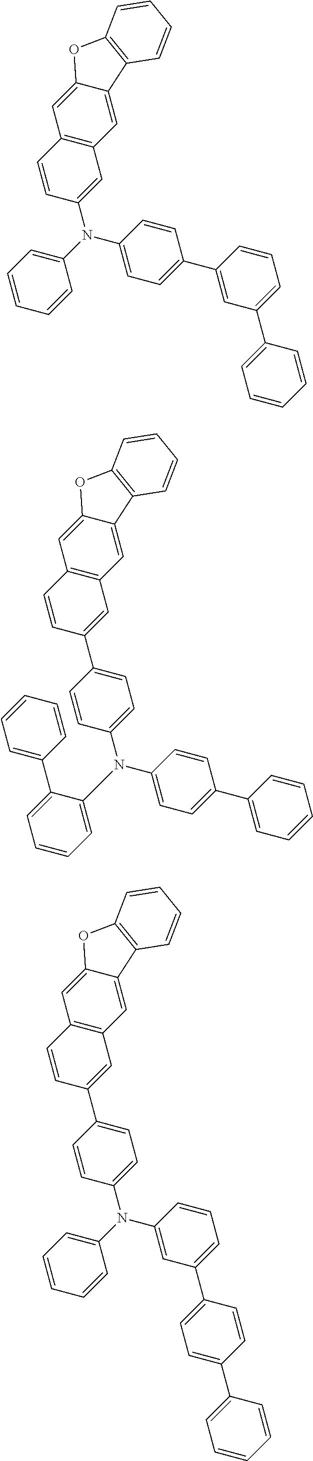 [SMILES: c1ccc(-c2cccc(-c3ccc(N(c4ccccc4)c4ccc5cc6oc7ccccc7c6cc5c4)cc3)c2)cc1 ; c1ccc(-c2ccc(N(c3ccc(-c4ccc5cc6oc7ccccc7c6cc5c4)cc3)c3ccccc3-c3ccccc3)cc2)cc1 ; c1ccc(-c2ccc(-c3cccc(N(c4ccccc4)c4ccc(-c5ccc6cc7oc8ccccc8c7cc6c5)cc4)c3)cc2)cc1] 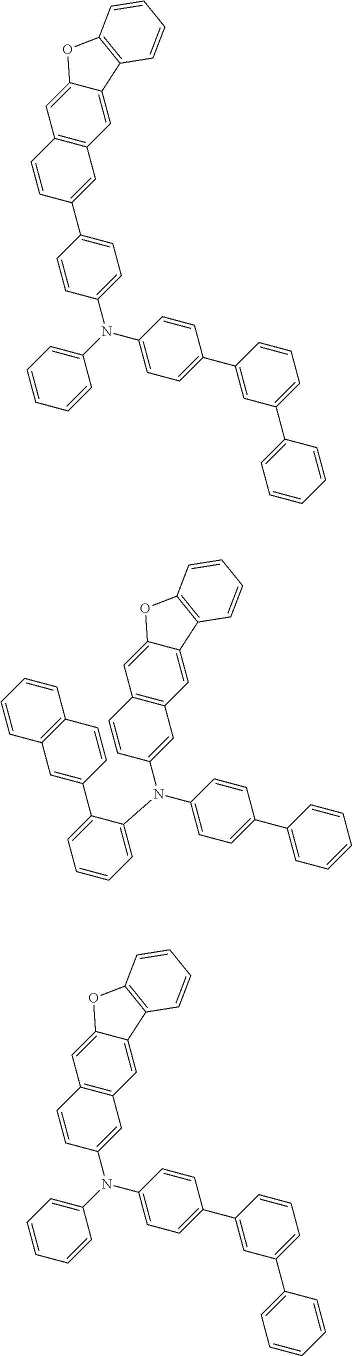 [SMILES: c1ccc(-c2cccc(-c3ccc(N(c4ccccc4)c4ccc(-c5ccc6cc7oc8ccccc8c7cc6c5)cc4)cc3)c2)cc1 ; c1ccc(-c2ccc(N(c3ccc4cc5oc6ccccc6c5cc4c3)c3ccccc3-c3ccc4ccccc4c3)cc2)cc1 ; c1ccc(-c2cccc(-c3ccc(N(c4ccccc4)c4ccc5cc6oc7ccccc7c6cc5c4)cc3)c2)cc1] 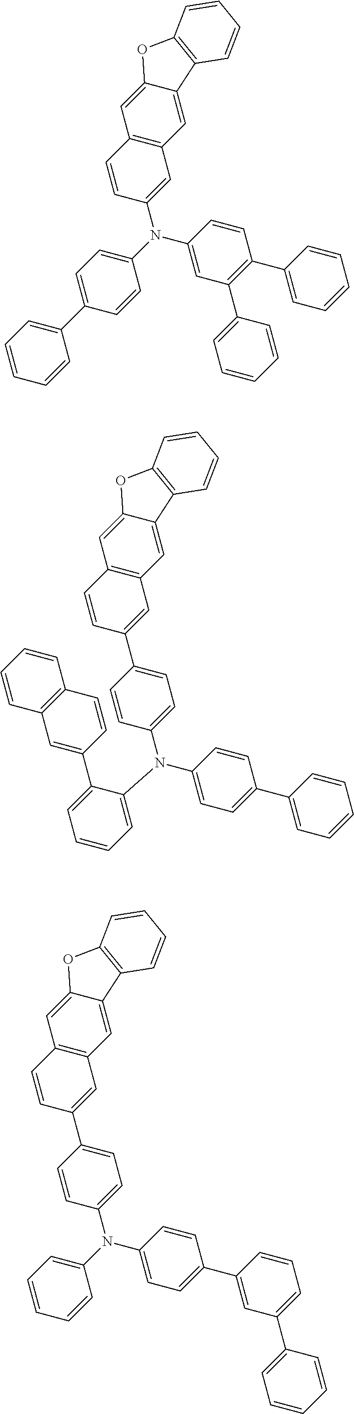 [SMILES: c1ccc(-c2ccc(N(c3ccc(-c4ccccc4)c(-c4ccccc4)c3)c3ccc4cc5oc6ccccc6c5cc4c3)cc2)cc1 ; c1ccc(-c2ccc(N(c3ccc(-c4ccc5cc6oc7ccccc7c6cc5c4)cc3)c3ccccc3-c3ccc4ccccc4c3)cc2)cc1 ; c1ccc(-c2cccc(-c3ccc(N(c4ccccc4)c4ccc(-c5ccc6cc7oc8ccccc8c7cc6c5)cc4)cc3)c2)cc1] 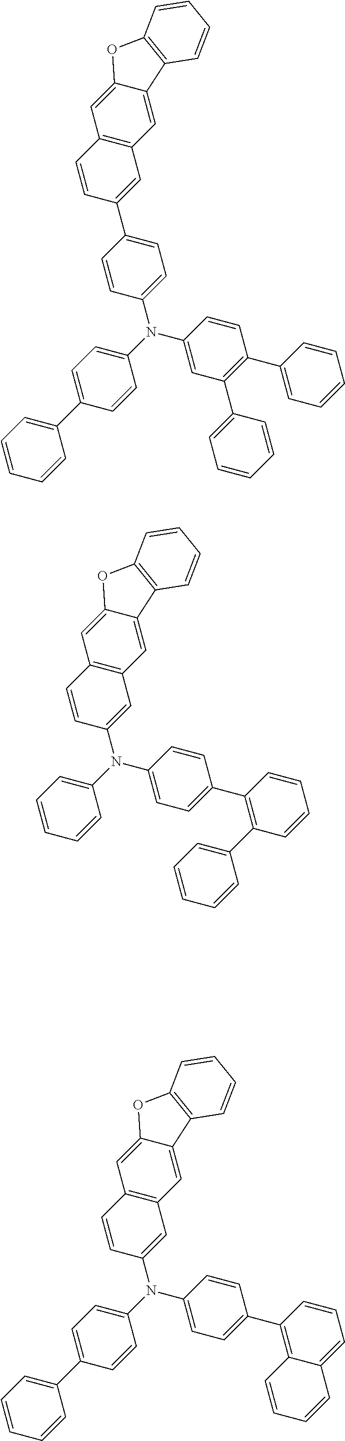 [SMILES: c1ccc(-c2ccc(N(c3ccc(-c4ccc5cc6oc7ccccc7c6cc5c4)cc3)c3ccc(-c4ccccc4)c(-c4ccccc4)c3)cc2)cc1 ; c1ccc(-c2ccccc2-c2ccc(N(c3ccccc3)c3ccc4cc5oc6ccccc6c5cc4c3)cc2)cc1 ; c1ccc(-c2ccc(N(c3ccc(-c4cccc5ccccc45)cc3)c3ccc4cc5oc6ccccc6c5cc4c3)cc2)cc1] 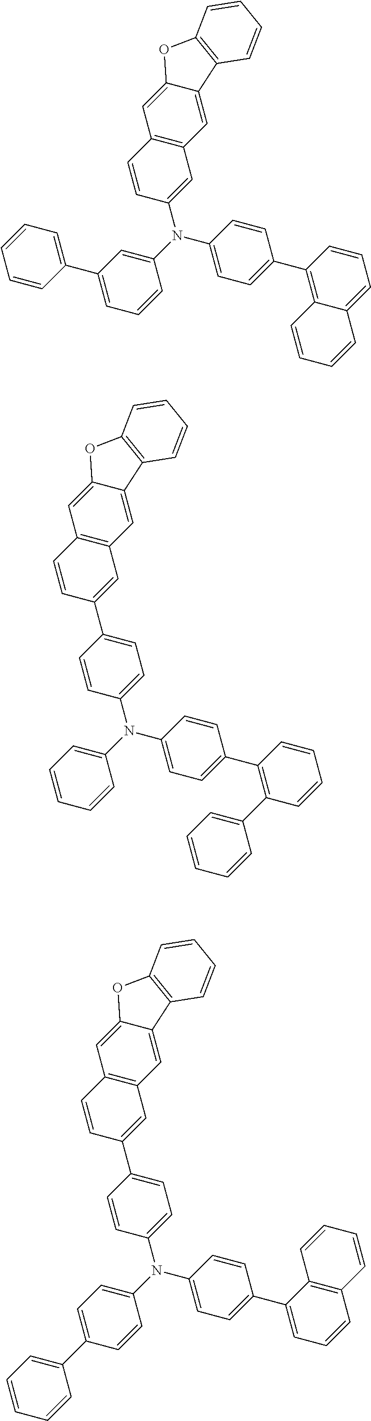 [SMILES: c1ccc(-c2cccc(N(c3ccc(-c4cccc5ccccc45)cc3)c3ccc4cc5oc6ccccc6c5cc4c3)c2)cc1 ; c1ccc(-c2ccccc2-c2ccc(N(c3ccccc3)c3ccc(-c4ccc5cc6oc7ccccc7c6cc5c4)cc3)cc2)cc1 ; c1ccc(-c2ccc(N(c3ccc(-c4ccc5cc6oc7ccccc7c6cc5c4)cc3)c3ccc(-c4cccc5ccccc45)cc3)cc2)cc1] 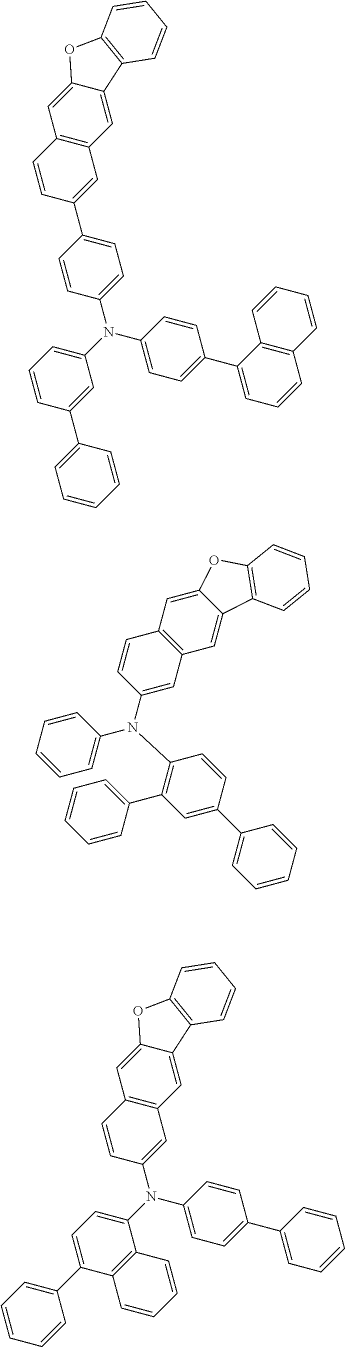 [SMILES: c1ccc(-c2cccc(N(c3ccc(-c4ccc5cc6oc7ccccc7c6cc5c4)cc3)c3ccc(-c4cccc5ccccc45)cc3)c2)cc1 ; c1ccc(-c2ccc(N(c3ccccc3)c3ccc4cc5oc6ccccc6c5cc4c3)c(-c3ccccc3)c2)cc1 ; c1ccc(-c2ccc(N(c3ccc4cc5oc6ccccc6c5cc4c3)c3ccc(-c4ccccc4)c4ccccc34)cc2)cc1] 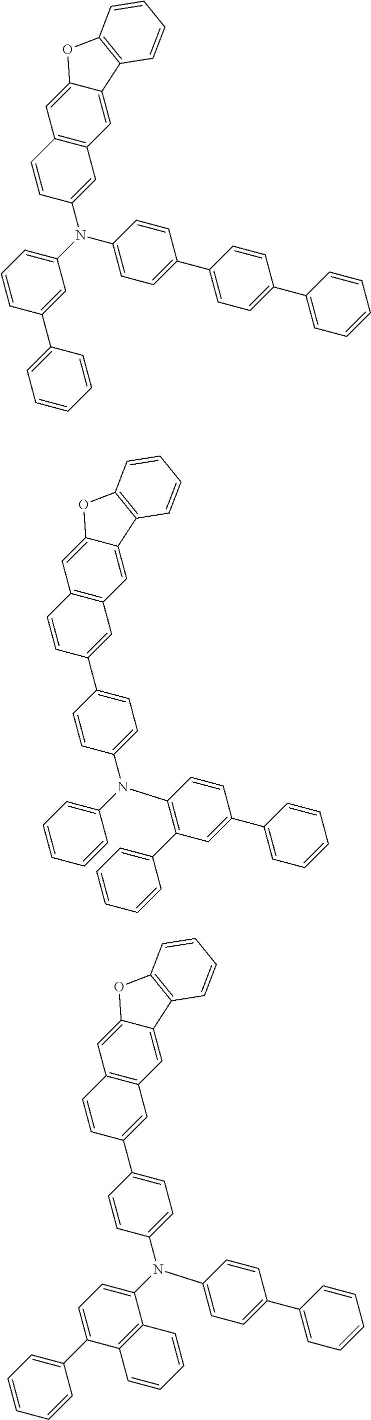 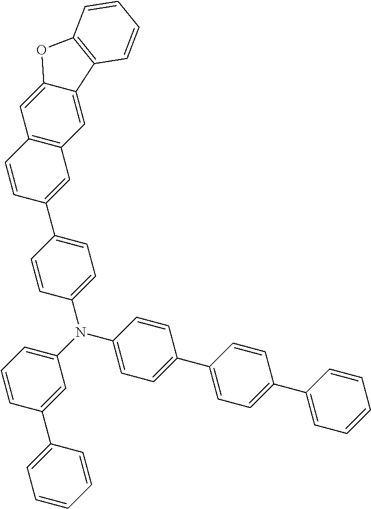 [SMILES: c1ccc(-c2ccc(-c3ccc(N(c4ccc(-c5ccc6cc7oc8ccccc8c7cc6c5)cc4)c4cccc(-c5ccccc5)c4)cc3)cc2)cc1] 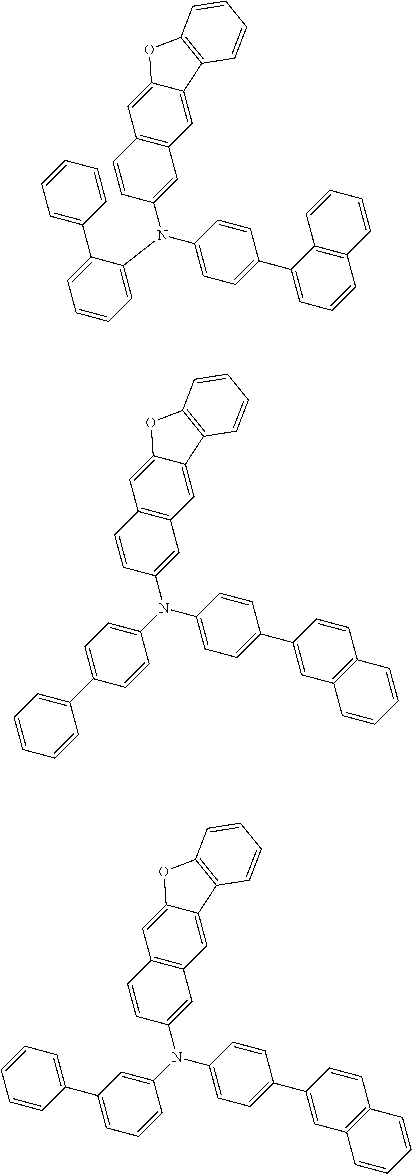 [SMILES: c1ccc(-c2ccccc2N(c2ccc(-c3cccc4ccccc34)cc2)c2ccc3cc4oc5ccccc5c4cc3c2)cc1 ; c1ccc(-c2ccc(N(c3ccc(-c4ccc5ccccc5c4)cc3)c3ccc4cc5oc6ccccc6c5cc4c3)cc2)cc1 ; c1ccc(-c2cccc(N(c3ccc(-c4ccc5ccccc5c4)cc3)c3ccc4cc5oc6ccccc6c5cc4c3)c2)cc1] 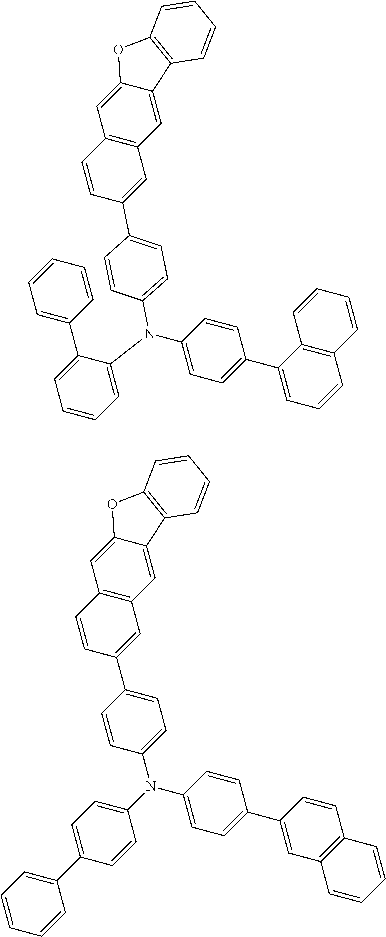 [SMILES: c1ccc(-c2ccccc2N(c2ccc(-c3ccc4cc5oc6ccccc6c5cc4c3)cc2)c2ccc(-c3cccc4ccccc34)cc2)cc1 ; c1ccc(-c2ccc(N(c3ccc(-c4ccc5ccccc5c4)cc3)c3ccc(-c4ccc5cc6oc7ccccc7c6cc5c4)cc3)cc2)cc1] 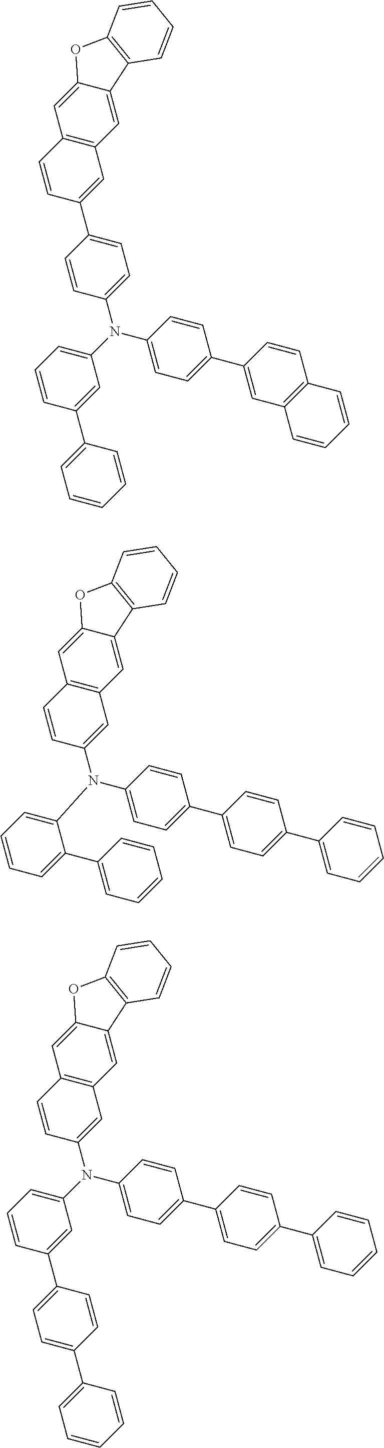 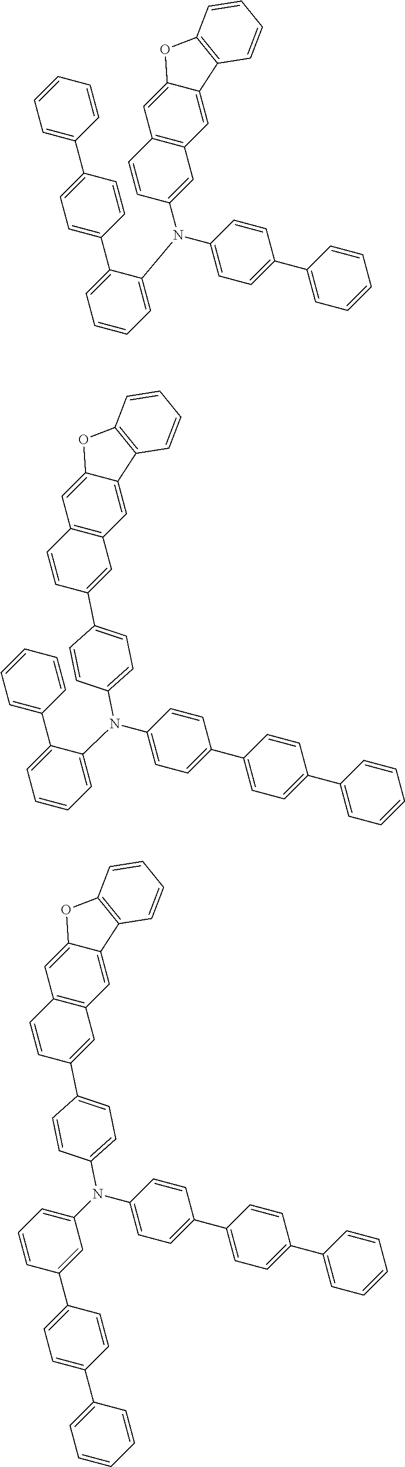 [SMILES: c1ccc(-c2ccc(-c3ccccc3N(c3ccc(-c4ccccc4)cc3)c3ccc4cc5oc6ccccc6c5cc4c3)cc2)cc1 ; c1ccc(-c2ccc(-c3ccc(N(c4ccc(-c5ccc6cc7oc8ccccc8c7cc6c5)cc4)c4ccccc4-c4ccccc4)cc3)cc2)cc1 ; c1ccc(-c2ccc(-c3ccc(N(c4ccc(-c5ccc6cc7oc8ccccc8c7cc6c5)cc4)c4cccc(-c5ccc(-c6ccccc6)cc5)c4)cc3)cc2)cc1] 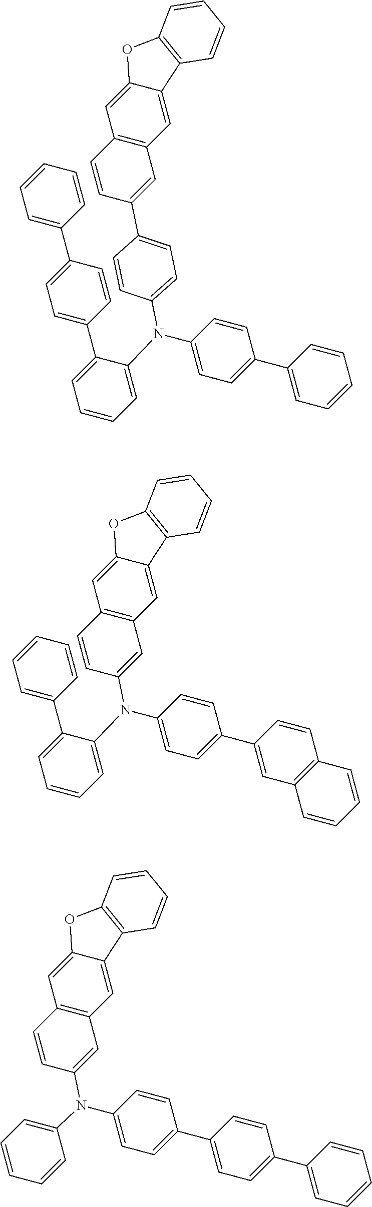 [SMILES: c1ccc(-c2ccc(-c3ccccc3N(c3ccc(-c4ccccc4)cc3)c3ccc(-c4ccc5cc6oc7ccccc7c6cc5c4)cc3)cc2)cc1 ; c1ccc(-c2ccccc2N(c2ccc(-c3ccc4ccccc4c3)cc2)c2ccc3cc4oc5ccccc5c4cc3c2)cc1 ; c1ccc(-c2ccc(-c3ccc(N(c4ccccc4)c4ccc5cc6oc7ccccc7c6cc5c4)cc3)cc2)cc1] 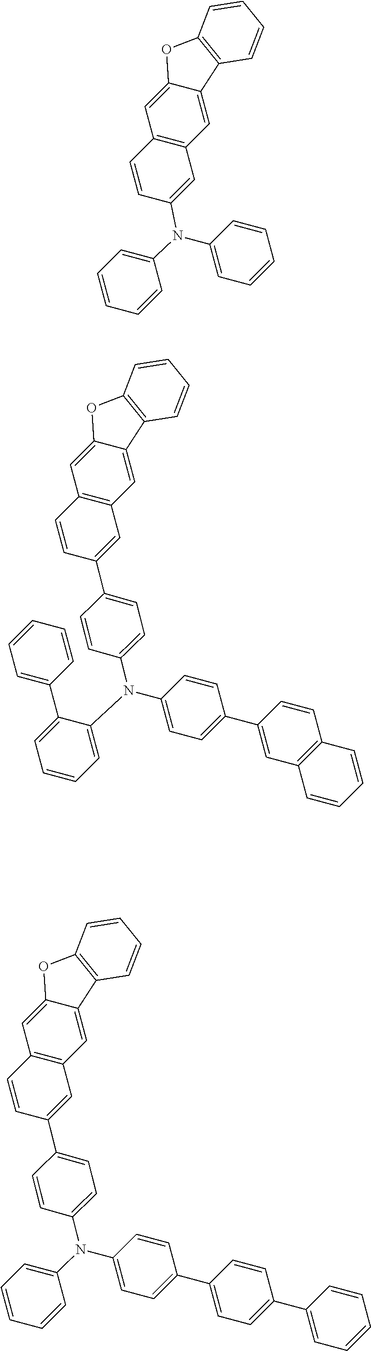 [SMILES: c1ccc(N(c2ccccc2)c2ccc3cc4oc5ccccc5c4cc3c2)cc1 ; c1ccc(-c2ccccc2N(c2ccc(-c3ccc4ccccc4c3)cc2)c2ccc(-c3ccc4cc5oc6ccccc6c5cc4c3)cc2)cc1 ; c1ccc(-c2ccc(-c3ccc(N(c4ccccc4)c4ccc(-c5ccc6cc7oc8ccccc8c7cc6c5)cc4)cc3)cc2)cc1] 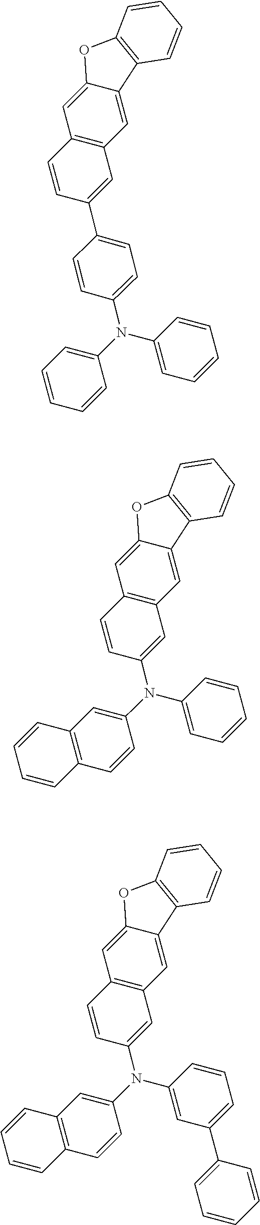 [SMILES: c1ccc(N(c2ccccc2)c2ccc(-c3ccc4cc5oc6ccccc6c5cc4c3)cc2)cc1 ; c1ccc(N(c2ccc3ccccc3c2)c2ccc3cc4oc5ccccc5c4cc3c2)cc1 ; c1ccc(-c2cccc(N(c3ccc4ccccc4c3)c3ccc4cc5oc6ccccc6c5cc4c3)c2)cc1] 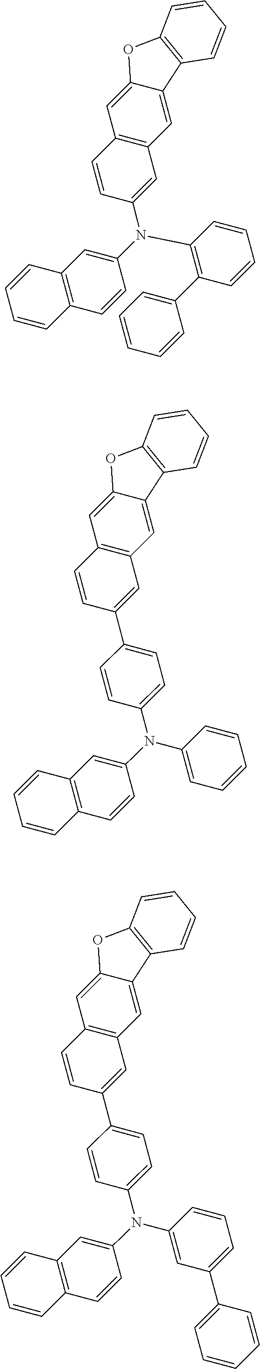 [SMILES: c1ccc(-c2ccccc2N(c2ccc3ccccc3c2)c2ccc3cc4oc5ccccc5c4cc3c2)cc1 ; c1ccc(N(c2ccc(-c3ccc4cc5oc6ccccc6c5cc4c3)cc2)c2ccc3ccccc3c2)cc1 ; c1ccc(-c2cccc(N(c3ccc(-c4ccc5cc6oc7ccccc7c6cc5c4)cc3)c3ccc4ccccc4c3)c2)cc1] 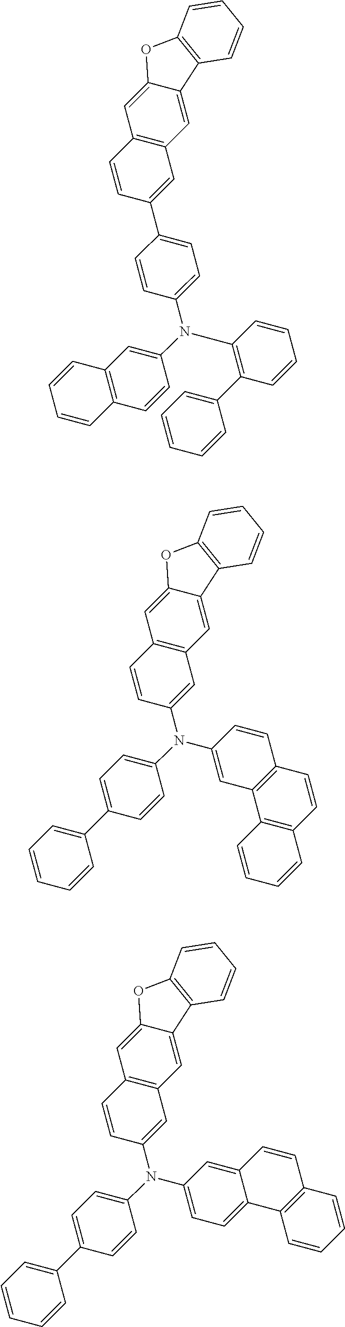 [SMILES: c1ccc(-c2ccccc2N(c2ccc(-c3ccc4cc5oc6ccccc6c5cc4c3)cc2)c2ccc3ccccc3c2)cc1 ; c1ccc(-c2ccc(N(c3ccc4cc5oc6ccccc6c5cc4c3)c3ccc4ccc5ccccc5c4c3)cc2)cc1 ; c1ccc(-c2ccc(N(c3ccc4cc5oc6ccccc6c5cc4c3)c3ccc4c(ccc5ccccc54)c3)cc2)cc1] 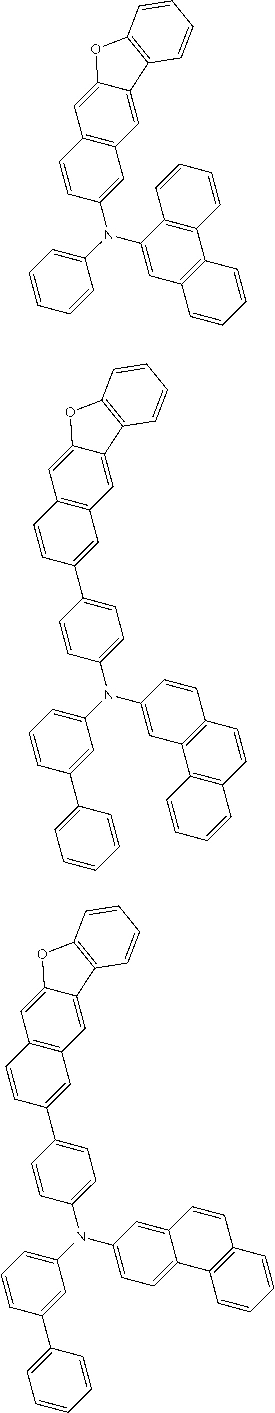 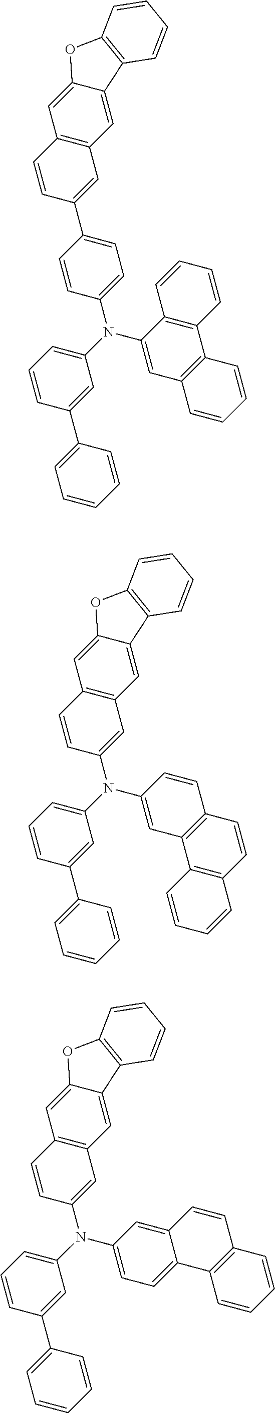 [SMILES: c1ccc(-c2cccc(N(c3ccc(-c4ccc5cc6oc7ccccc7c6cc5c4)cc3)c3cc4ccccc4c4ccccc34)c2)cc1 ; c1ccc(-c2cccc(N(c3ccc4cc5oc6ccccc6c5cc4c3)c3ccc4ccc5ccccc5c4c3)c2)cc1 ; c1ccc(-c2cccc(N(c3ccc4cc5oc6ccccc6c5cc4c3)c3ccc4c(ccc5ccccc54)c3)c2)cc1] 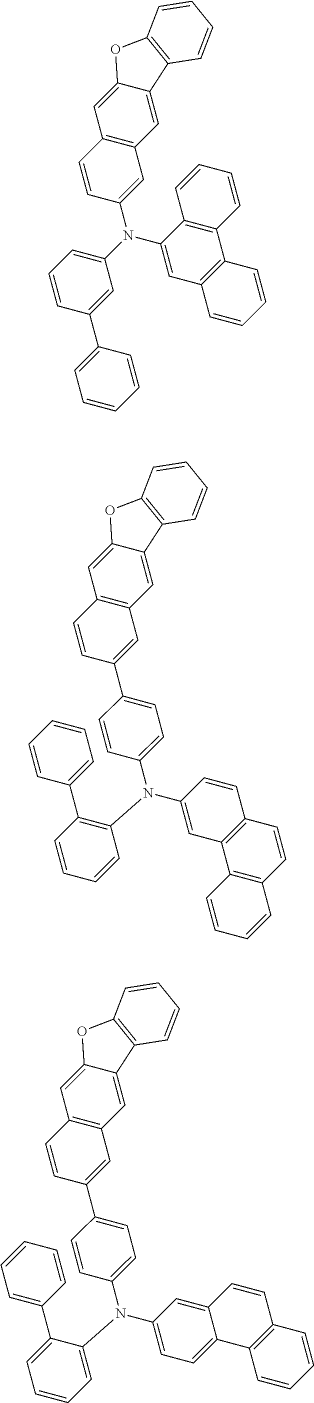 [SMILES: c1ccc(-c2cccc(N(c3ccc4cc5oc6ccccc6c5cc4c3)c3cc4ccccc4c4ccccc34)c2)cc1 ; c1ccc(-c2ccccc2N(c2ccc(-c3ccc4cc5oc6ccccc6c5cc4c3)cc2)c2ccc3ccc4ccccc4c3c2)cc1 ; c1ccc(-c2ccccc2N(c2ccc(-c3ccc4cc5oc6ccccc6c5cc4c3)cc2)c2ccc3c(ccc4ccccc43)c2)cc1] 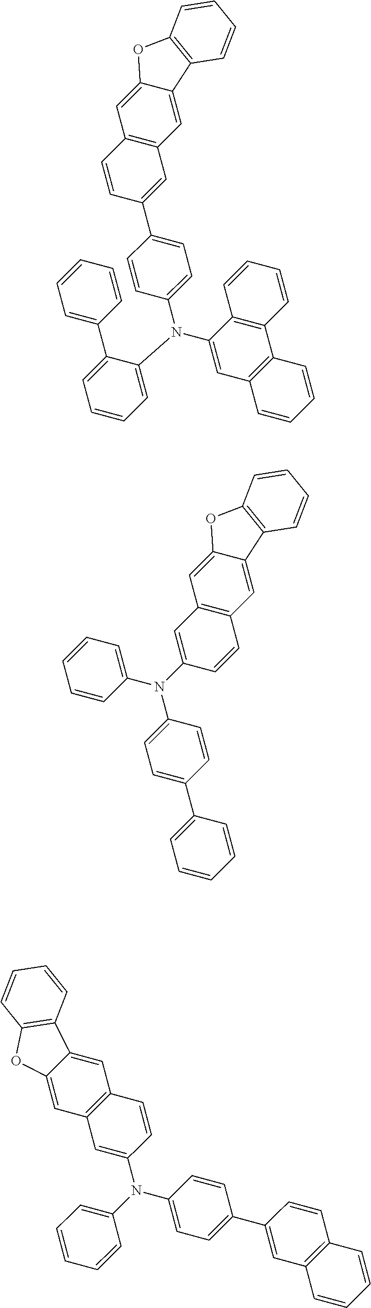 [SMILES: c1ccc(-c2ccccc2N(c2ccc(-c3ccc4cc5oc6ccccc6c5cc4c3)cc2)c2cc3ccccc3c3ccccc23)cc1 ; c1ccc(-c2ccc(N(c3ccccc3)c3ccc4cc5c(cc4c3)oc3ccccc35)cc2)cc1 ; c1ccc(N(c2ccc(-c3ccc4ccccc4c3)cc2)c2ccc3cc4c(cc3c2)oc2ccccc24)cc1] 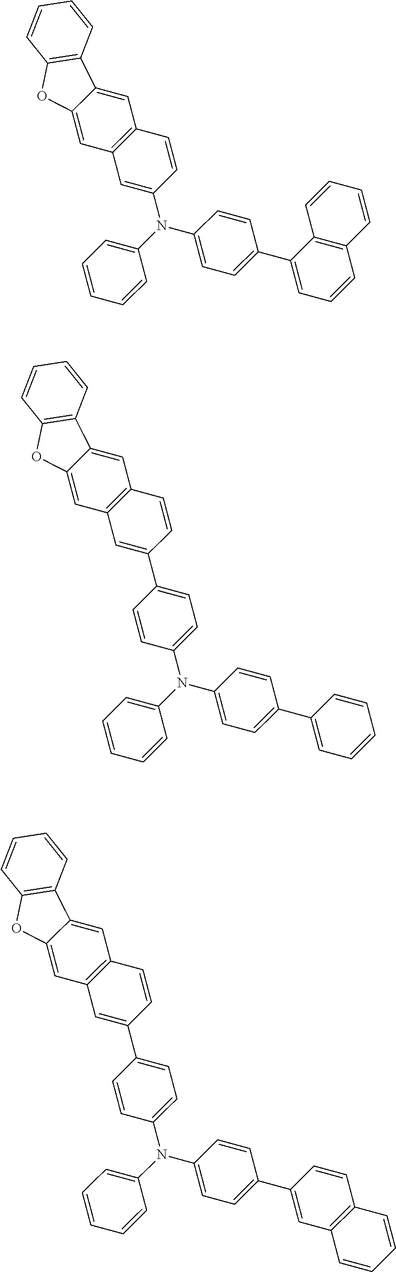 [SMILES: c1ccc(N(c2ccc(-c3cccc4ccccc34)cc2)c2ccc3cc4c(cc3c2)oc2ccccc24)cc1 ; c1ccc(-c2ccc(N(c3ccccc3)c3ccc(-c4ccc5cc6c(cc5c4)oc4ccccc46)cc3)cc2)cc1 ; c1ccc(N(c2ccc(-c3ccc4ccccc4c3)cc2)c2ccc(-c3ccc4cc5c(cc4c3)oc3ccccc35)cc2)cc1] 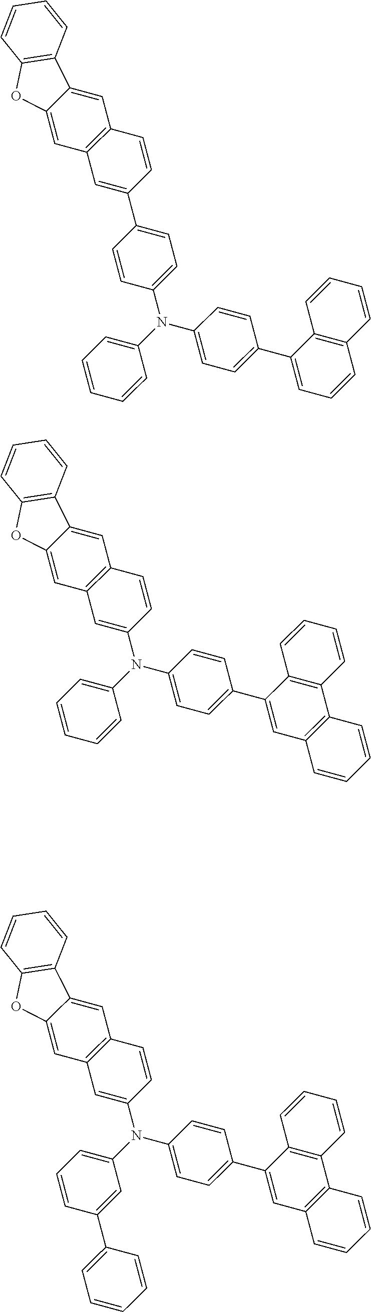 [SMILES: c1ccc(N(c2ccc(-c3ccc4cc5c(cc4c3)oc3ccccc35)cc2)c2ccc(-c3cccc4ccccc34)cc2)cc1 ; c1ccc(N(c2ccc(-c3cc4ccccc4c4ccccc34)cc2)c2ccc3cc4c(cc3c2)oc2ccccc24)cc1 ; c1ccc(-c2cccc(N(c3ccc(-c4cc5ccccc5c5ccccc45)cc3)c3ccc4cc5c(cc4c3)oc3ccccc35)c2)cc1] 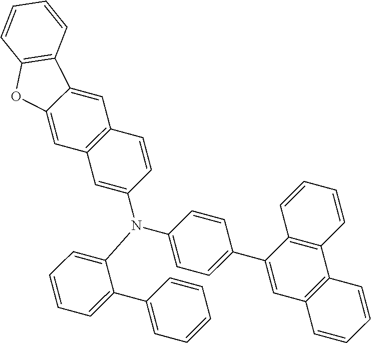 [SMILES: c1ccc(-c2ccccc2N(c2ccc(-c3cc4ccccc4c4ccccc34)cc2)c2ccc3cc4c(cc3c2)oc2ccccc24)cc1] 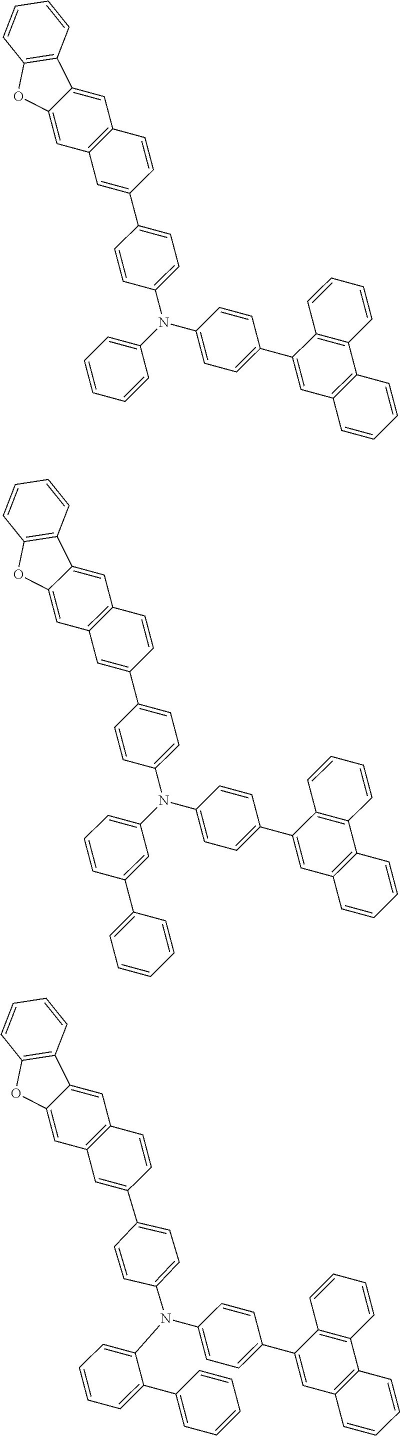 [SMILES: c1ccc(N(c2ccc(-c3ccc4cc5c(cc4c3)oc3ccccc35)cc2)c2ccc(-c3cc4ccccc4c4ccccc34)cc2)cc1 ; c1ccc(-c2cccc(N(c3ccc(-c4ccc5cc6c(cc5c4)oc4ccccc46)cc3)c3ccc(-c4cc5ccccc5c5ccccc45)cc3)c2)cc1 ; c1ccc(-c2ccccc2N(c2ccc(-c3ccc4cc5c(cc4c3)oc3ccccc35)cc2)c2ccc(-c3cc4ccccc4c4ccccc34)cc2)cc1] 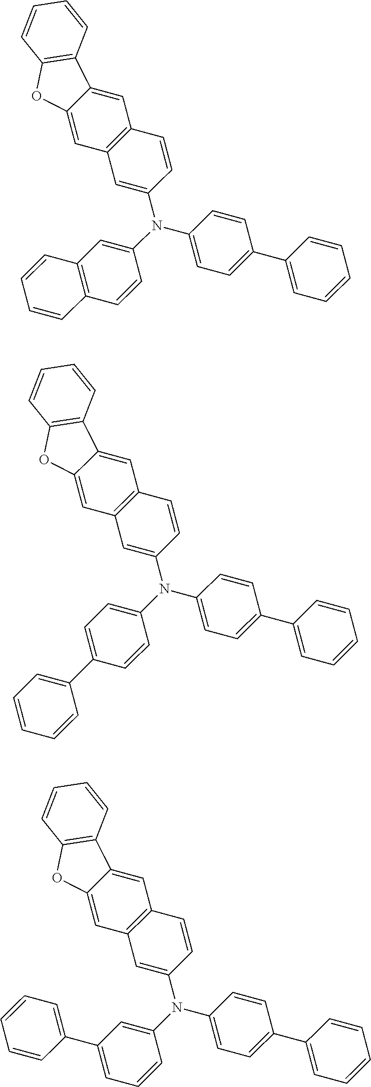 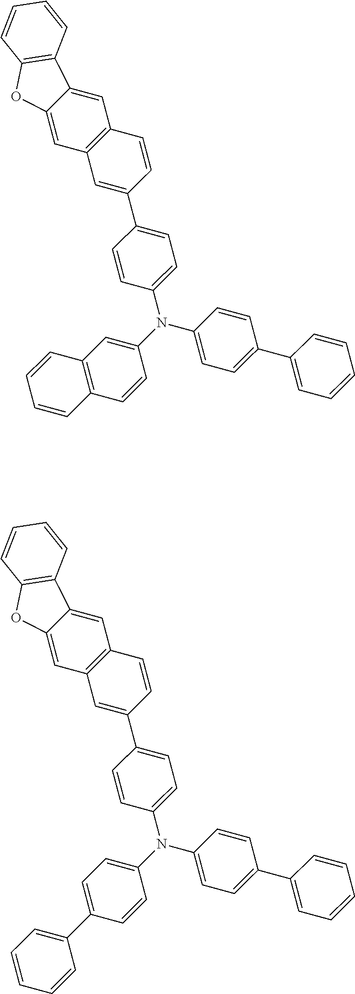 [SMILES: c1ccc(-c2ccc(N(c3ccc(-c4ccc5cc6c(cc5c4)oc4ccccc46)cc3)c3ccc4ccccc4c3)cc2)cc1 ; c1ccc(-c2ccc(N(c3ccc(-c4ccccc4)cc3)c3ccc(-c4ccc5cc6c(cc5c4)oc4ccccc46)cc3)cc2)cc1] 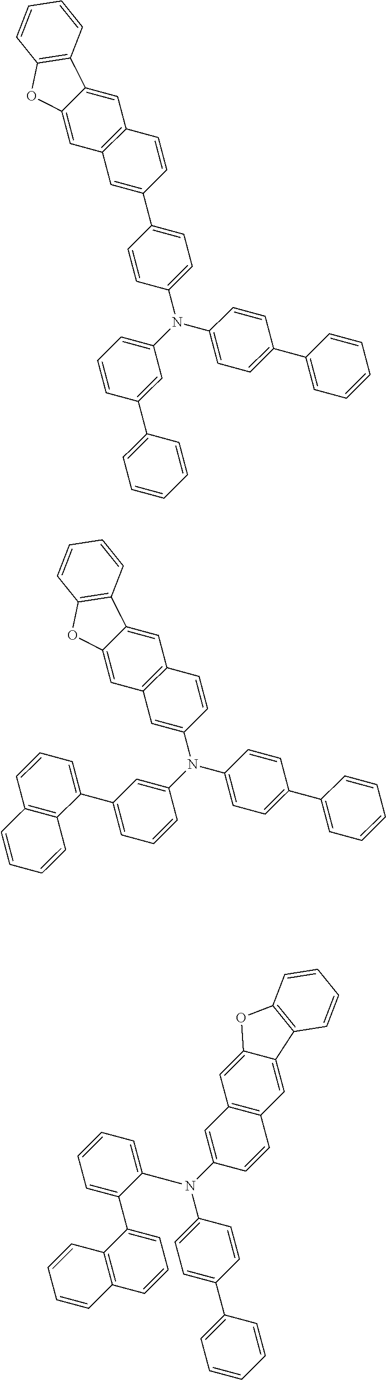 [SMILES: c1ccc(-c2ccc(N(c3ccc(-c4ccc5cc6c(cc5c4)oc4ccccc46)cc3)c3cccc(-c4ccccc4)c3)cc2)cc1 ; c1ccc(-c2ccc(N(c3cccc(-c4cccc5ccccc45)c3)c3ccc4cc5c(cc4c3)oc3ccccc35)cc2)cc1 ; c1ccc(-c2ccc(N(c3ccc4cc5c(cc4c3)oc3ccccc35)c3ccccc3-c3cccc4ccccc34)cc2)cc1] 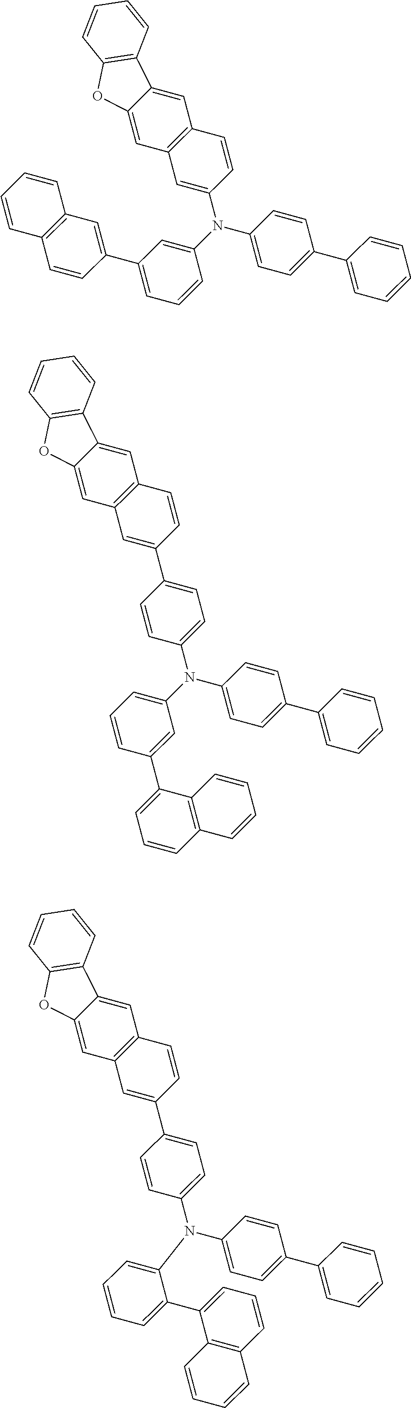 [SMILES: c1ccc(-c2ccc(N(c3cccc(-c4ccc5ccccc5c4)c3)c3ccc4cc5c(cc4c3)oc3ccccc35)cc2)cc1 ; c1ccc(-c2ccc(N(c3ccc(-c4ccc5cc6c(cc5c4)oc4ccccc46)cc3)c3cccc(-c4cccc5ccccc45)c3)cc2)cc1 ; c1ccc(-c2ccc(N(c3ccc(-c4ccc5cc6c(cc5c4)oc4ccccc46)cc3)c3ccccc3-c3cccc4ccccc34)cc2)cc1] 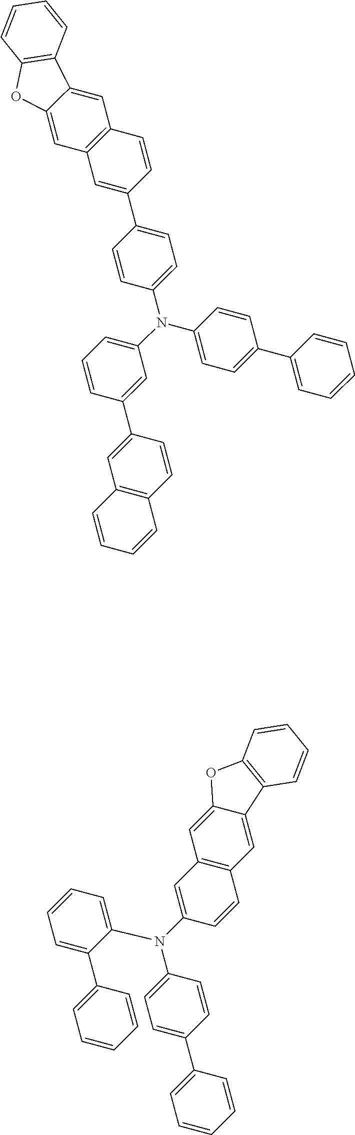 [SMILES: c1ccc(-c2ccc(N(c3ccc(-c4ccc5cc6c(cc5c4)oc4ccccc46)cc3)c3cccc(-c4ccc5ccccc5c4)c3)cc2)cc1 ; c1ccc(-c2ccc(N(c3ccc4cc5c(cc4c3)oc3ccccc35)c3ccccc3-c3ccccc3)cc2)cc1] 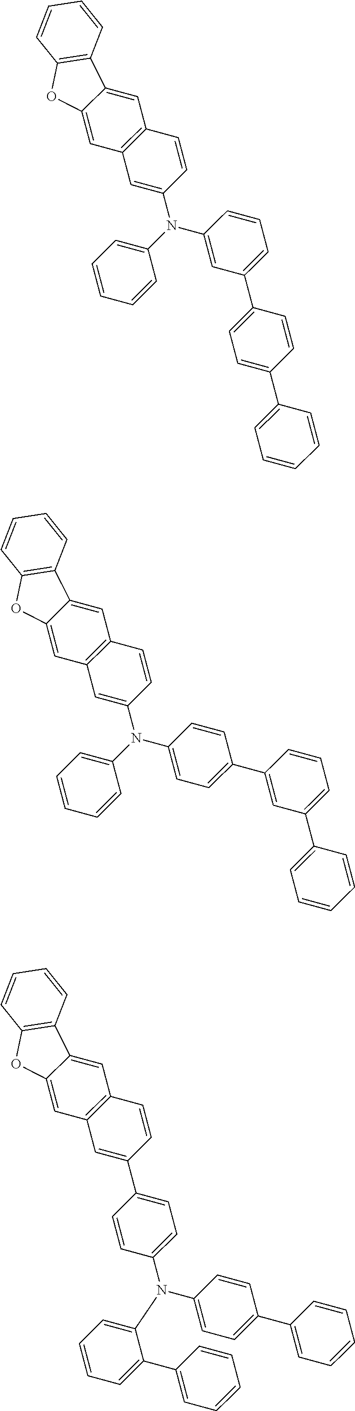 [SMILES: c1ccc(-c2ccc(-c3cccc(N(c4ccccc4)c4ccc5cc6c(cc5c4)oc4ccccc46)c3)cc2)cc1 ; c1ccc(-c2cccc(-c3ccc(N(c4ccccc4)c4ccc5cc6c(cc5c4)oc4ccccc46)cc3)c2)cc1 ; c1ccc(-c2ccc(N(c3ccc(-c4ccc5cc6c(cc5c4)oc4ccccc46)cc3)c3ccccc3-c3ccccc3)cc2)cc1] 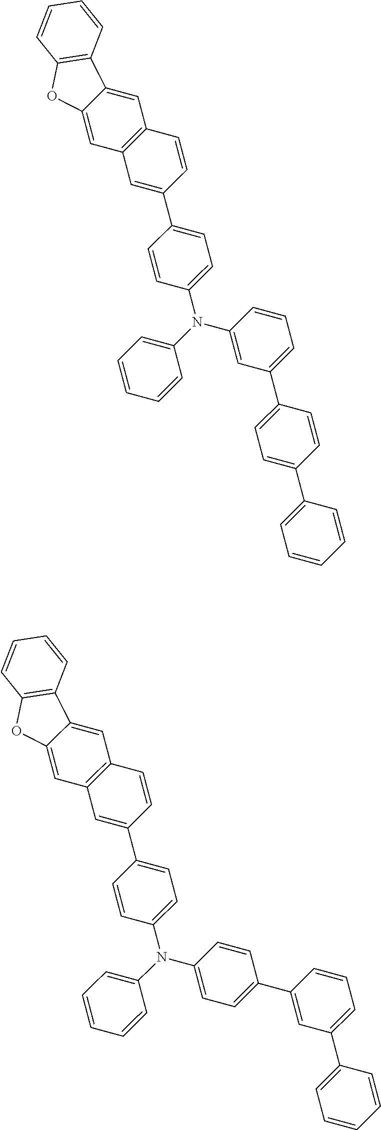 [SMILES: c1ccc(-c2ccc(-c3cccc(N(c4ccccc4)c4ccc(-c5ccc6cc7c(cc6c5)oc5ccccc57)cc4)c3)cc2)cc1 ; c1ccc(-c2cccc(-c3ccc(N(c4ccccc4)c4ccc(-c5ccc6cc7c(cc6c5)oc5ccccc57)cc4)cc3)c2)cc1] 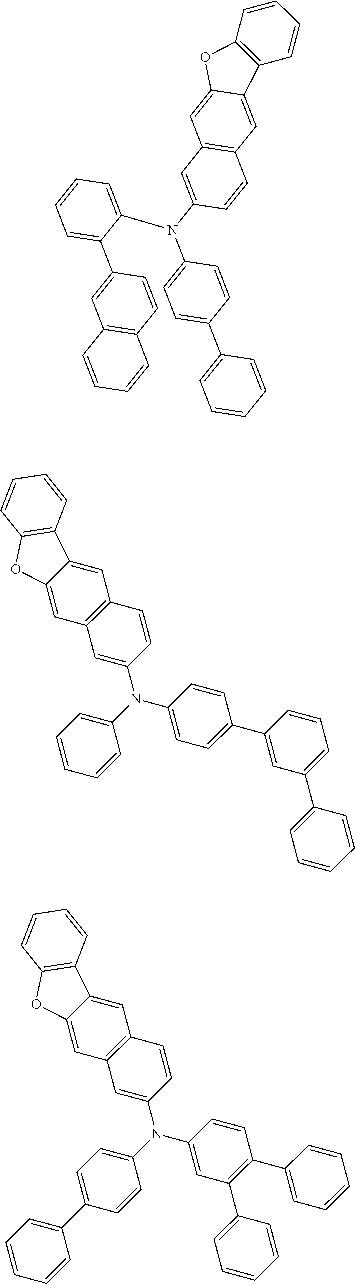 [SMILES: c1ccc(-c2ccc(N(c3ccc4cc5c(cc4c3)oc3ccccc35)c3ccccc3-c3ccc4ccccc4c3)cc2)cc1 ; c1ccc(-c2cccc(-c3ccc(N(c4ccccc4)c4ccc5cc6c(cc5c4)oc4ccccc46)cc3)c2)cc1 ; c1ccc(-c2ccc(N(c3ccc(-c4ccccc4)c(-c4ccccc4)c3)c3ccc4cc5c(cc4c3)oc3ccccc35)cc2)cc1] 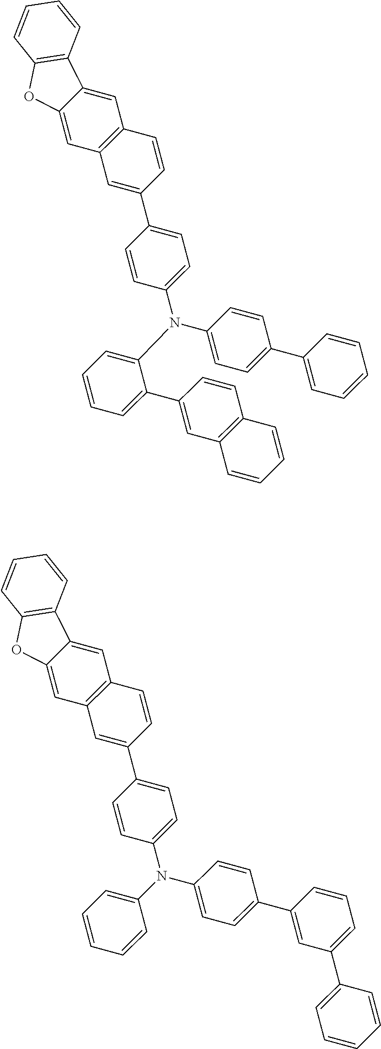 [SMILES: c1ccc(-c2ccc(N(c3ccc(-c4ccc5cc6c(cc5c4)oc4ccccc46)cc3)c3ccccc3-c3ccc4ccccc4c3)cc2)cc1 ; c1ccc(-c2cccc(-c3ccc(N(c4ccccc4)c4ccc(-c5ccc6cc7c(cc6c5)oc5ccccc57)cc4)cc3)c2)cc1] 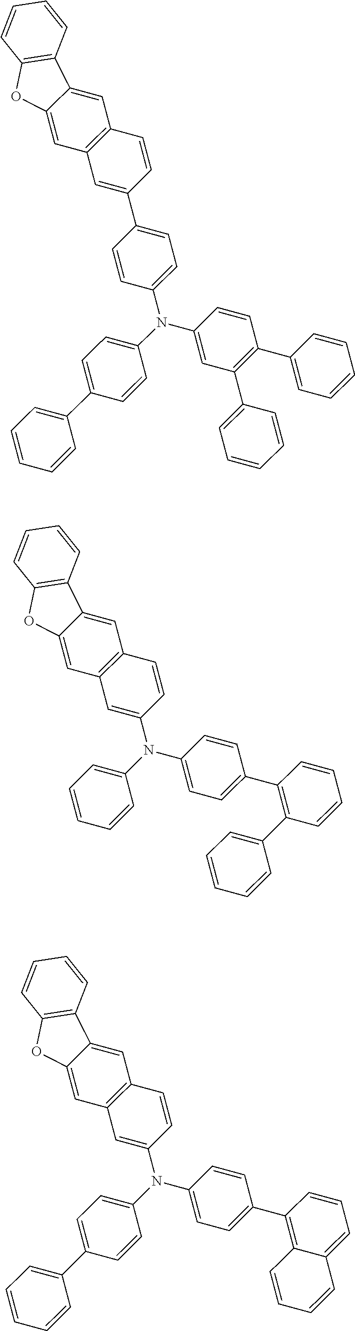 [SMILES: c1ccc(-c2ccc(N(c3ccc(-c4ccc5cc6c(cc5c4)oc4ccccc46)cc3)c3ccc(-c4ccccc4)c(-c4ccccc4)c3)cc2)cc1 ; c1ccc(-c2ccccc2-c2ccc(N(c3ccccc3)c3ccc4cc5c(cc4c3)oc3ccccc35)cc2)cc1 ; c1ccc(-c2ccc(N(c3ccc(-c4cccc5ccccc45)cc3)c3ccc4cc5c(cc4c3)oc3ccccc35)cc2)cc1] 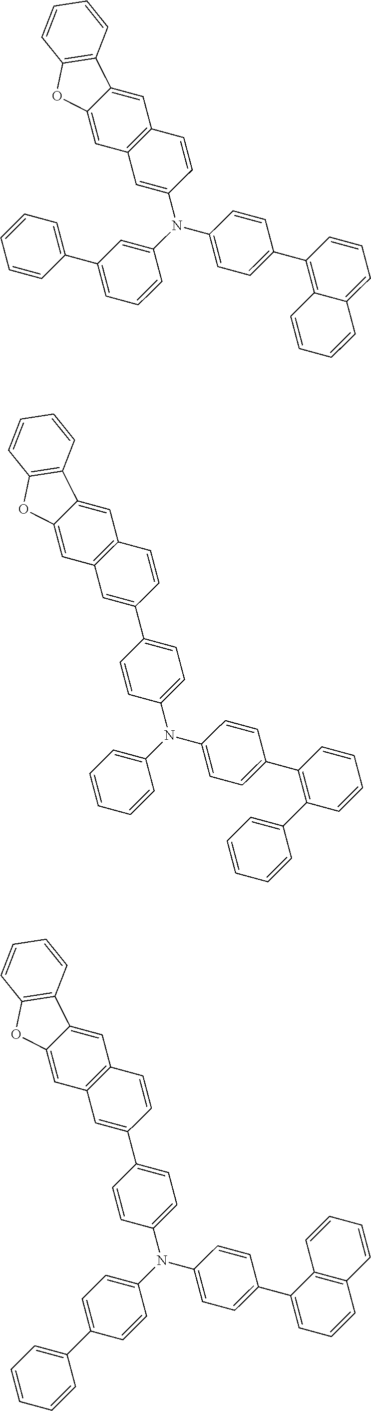 [SMILES: c1ccc(-c2cccc(N(c3ccc(-c4cccc5ccccc45)cc3)c3ccc4cc5c(cc4c3)oc3ccccc35)c2)cc1 ; c1ccc(-c2ccccc2-c2ccc(N(c3ccccc3)c3ccc(-c4ccc5cc6c(cc5c4)oc4ccccc46)cc3)cc2)cc1 ; c1ccc(-c2ccc(N(c3ccc(-c4ccc5cc6c(cc5c4)oc4ccccc46)cc3)c3ccc(-c4cccc5ccccc45)cc3)cc2)cc1] 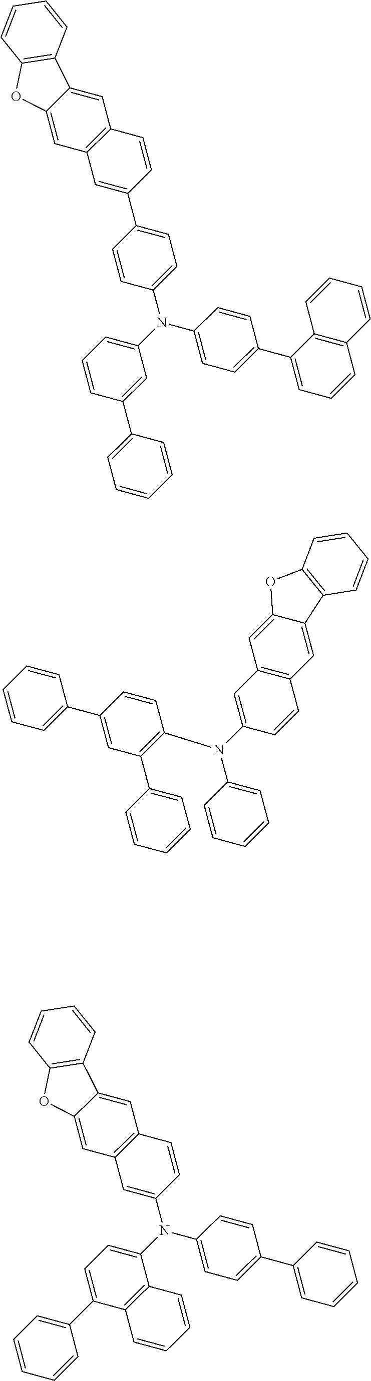 [SMILES: c1ccc(-c2cccc(N(c3ccc(-c4ccc5cc6c(cc5c4)oc4ccccc46)cc3)c3ccc(-c4cccc5ccccc45)cc3)c2)cc1 ; c1ccc(-c2ccc(N(c3ccccc3)c3ccc4cc5c(cc4c3)oc3ccccc35)c(-c3ccccc3)c2)cc1 ; c1ccc(-c2ccc(N(c3ccc4cc5c(cc4c3)oc3ccccc35)c3ccc(-c4ccccc4)c4ccccc34)cc2)cc1] 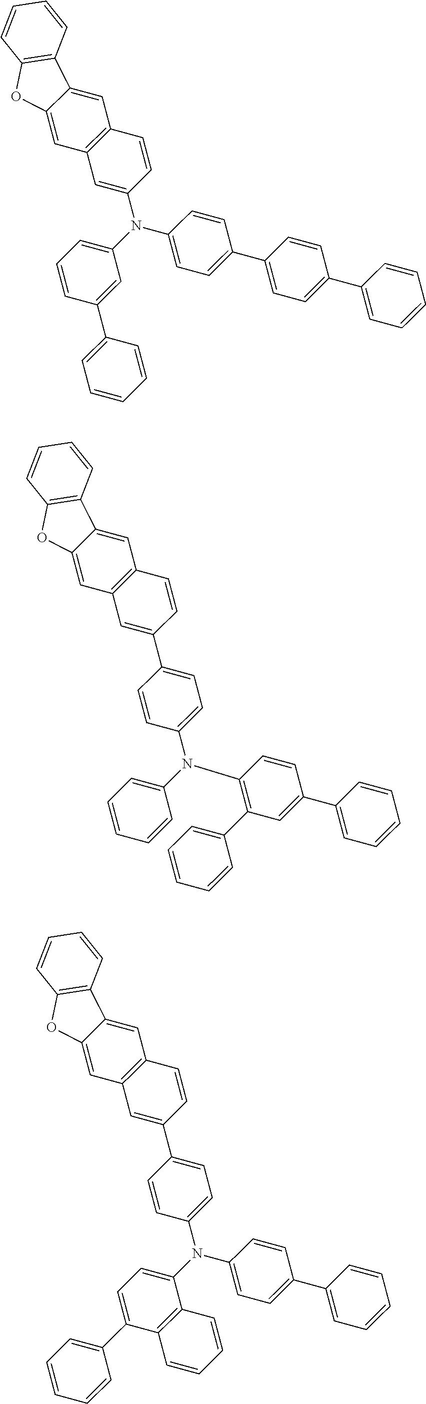 [SMILES: c1ccc(-c2ccc(-c3ccc(N(c4cccc(-c5ccccc5)c4)c4ccc5cc6c(cc5c4)oc4ccccc46)cc3)cc2)cc1 ; c1ccc(-c2ccc(N(c3ccccc3)c3ccc(-c4ccc5cc6c(cc5c4)oc4ccccc46)cc3)c(-c3ccccc3)c2)cc1 ; c1ccc(-c2ccc(N(c3ccc(-c4ccc5cc6c(cc5c4)oc4ccccc46)cc3)c3ccc(-c4ccccc4)c4ccccc34)cc2)cc1] 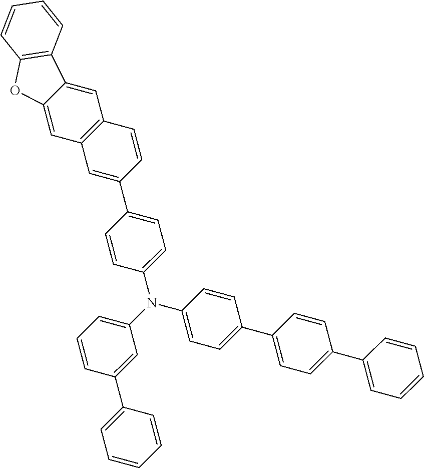 [SMILES: c1ccc(-c2ccc(-c3ccc(N(c4ccc(-c5ccc6cc7c(cc6c5)oc5ccccc57)cc4)c4cccc(-c5ccccc5)c4)cc3)cc2)cc1] 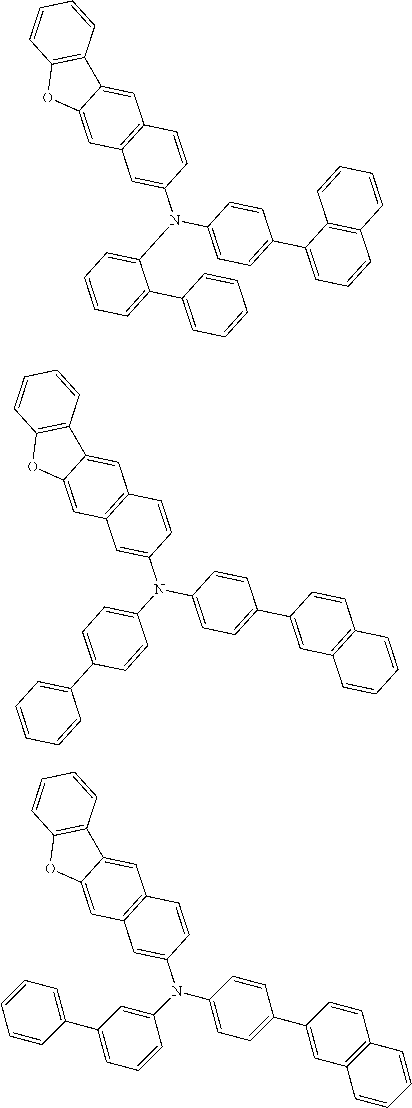 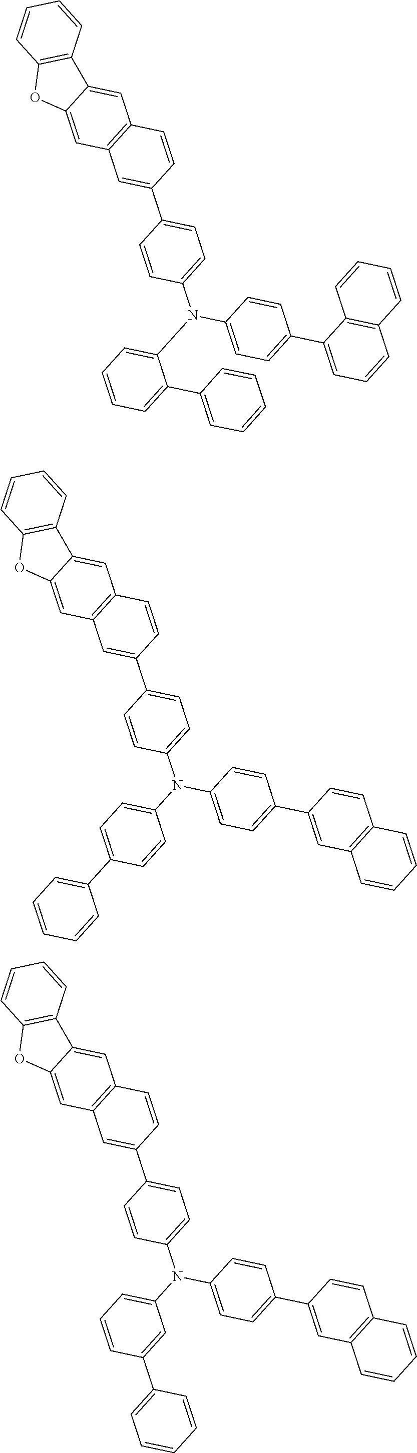 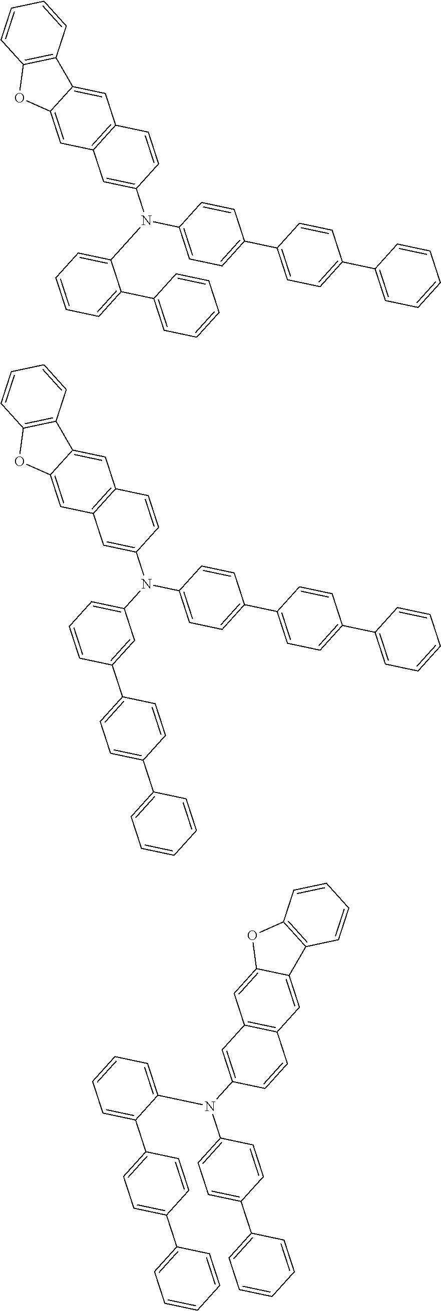 [SMILES: c1ccc(-c2ccc(-c3ccc(N(c4ccc5cc6c(cc5c4)oc4ccccc46)c4ccccc4-c4ccccc4)cc3)cc2)cc1 ; c1ccc(-c2ccc(-c3ccc(N(c4cccc(-c5ccc(-c6ccccc6)cc5)c4)c4ccc5cc6c(cc5c4)oc4ccccc46)cc3)cc2)cc1 ; c1ccc(-c2ccc(-c3ccccc3N(c3ccc(-c4ccccc4)cc3)c3ccc4cc5c(cc4c3)oc3ccccc35)cc2)cc1] 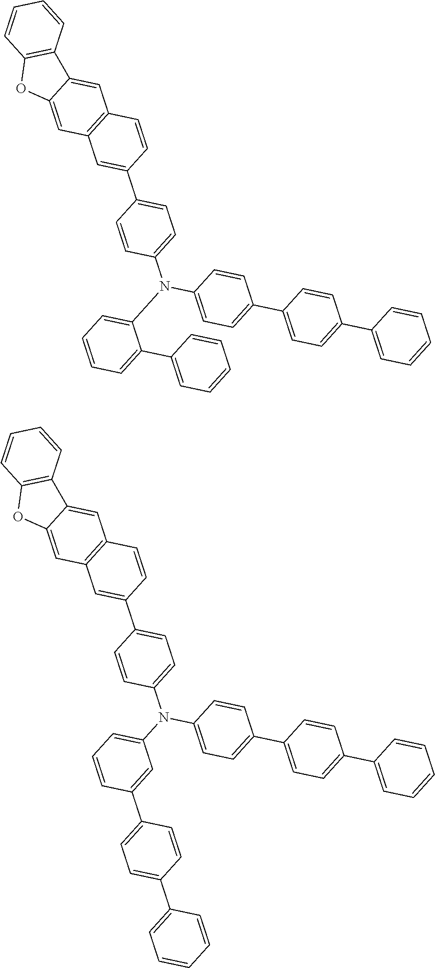 [SMILES: c1ccc(-c2ccc(-c3ccc(N(c4ccc(-c5ccc6cc7c(cc6c5)oc5ccccc57)cc4)c4ccccc4-c4ccccc4)cc3)cc2)cc1 ; c1ccc(-c2ccc(-c3ccc(N(c4ccc(-c5ccc6cc7c(cc6c5)oc5ccccc57)cc4)c4cccc(-c5ccc(-c6ccccc6)cc5)c4)cc3)cc2)cc1] 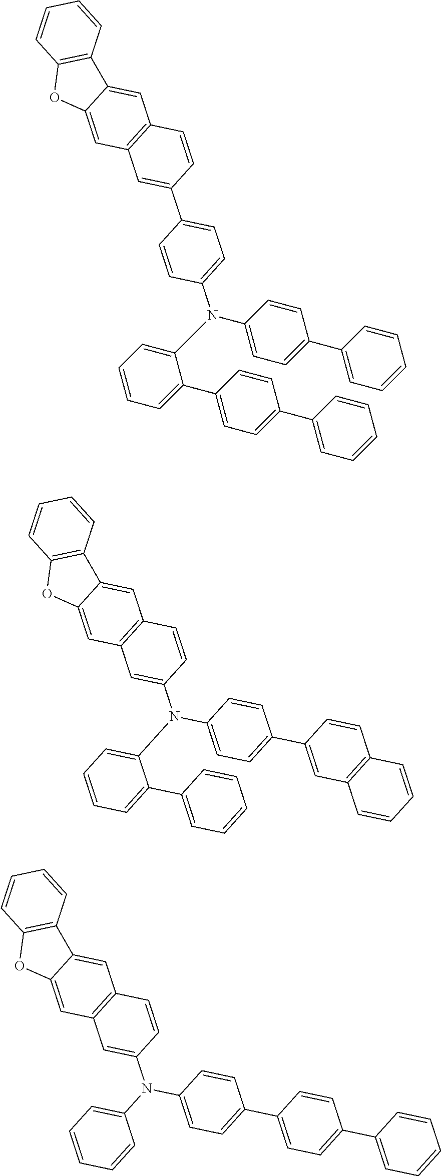 [SMILES: c1ccc(-c2ccc(-c3ccccc3N(c3ccc(-c4ccccc4)cc3)c3ccc(-c4ccc5cc6c(cc5c4)oc4ccccc46)cc3)cc2)cc1 ; c1ccc(-c2ccccc2N(c2ccc(-c3ccc4ccccc4c3)cc2)c2ccc3cc4c(cc3c2)oc2ccccc24)cc1 ; c1ccc(-c2ccc(-c3ccc(N(c4ccccc4)c4ccc5cc6c(cc5c4)oc4ccccc46)cc3)cc2)cc1] 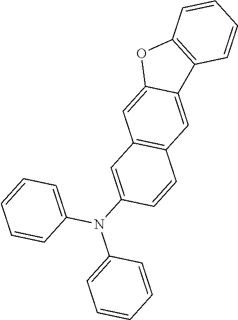 [SMILES: c1ccc(N(c2ccccc2)c2ccc3cc4c(cc3c2)oc2ccccc24)cc1] 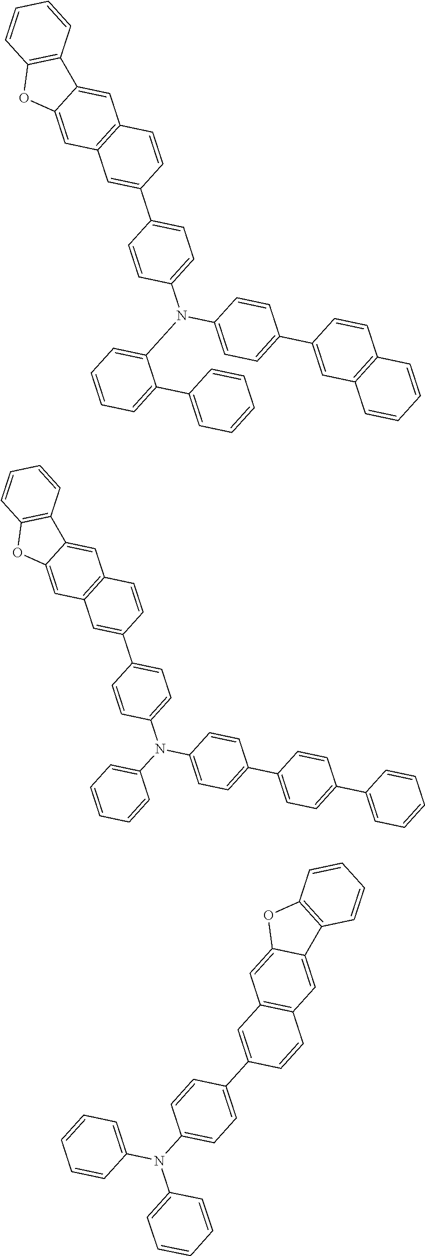 [SMILES: c1ccc(-c2ccccc2N(c2ccc(-c3ccc4ccccc4c3)cc2)c2ccc(-c3ccc4cc5c(cc4c3)oc3ccccc35)cc2)cc1 ; c1ccc(-c2ccc(-c3ccc(N(c4ccccc4)c4ccc(-c5ccc6cc7c(cc6c5)oc5ccccc57)cc4)cc3)cc2)cc1 ; c1ccc(N(c2ccccc2)c2ccc(-c3ccc4cc5c(cc4c3)oc3ccccc35)cc2)cc1] 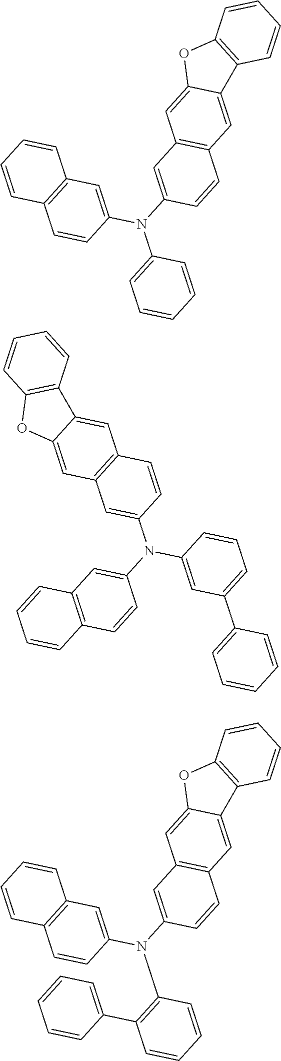 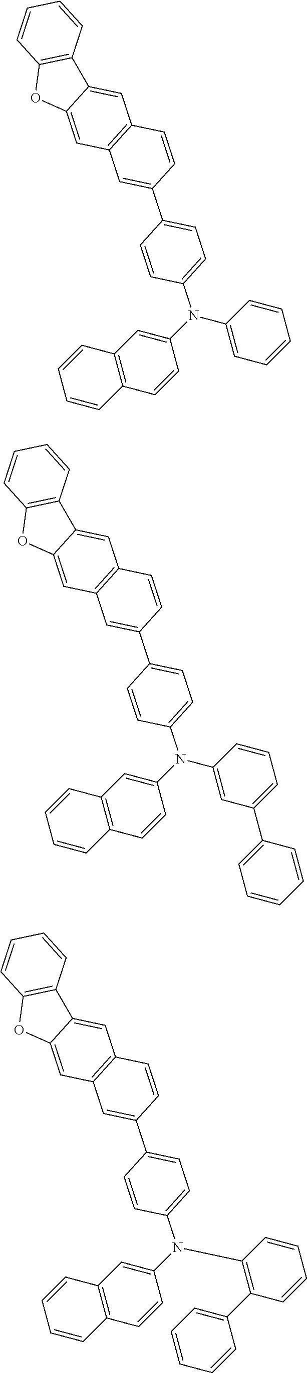 [SMILES: c1ccc(N(c2ccc(-c3ccc4cc5c(cc4c3)oc3ccccc35)cc2)c2ccc3ccccc3c2)cc1 ; c1ccc(-c2cccc(N(c3ccc(-c4ccc5cc6c(cc5c4)oc4ccccc46)cc3)c3ccc4ccccc4c3)c2)cc1 ; c1ccc(-c2ccccc2N(c2ccc(-c3ccc4cc5c(cc4c3)oc3ccccc35)cc2)c2ccc3ccccc3c2)cc1] 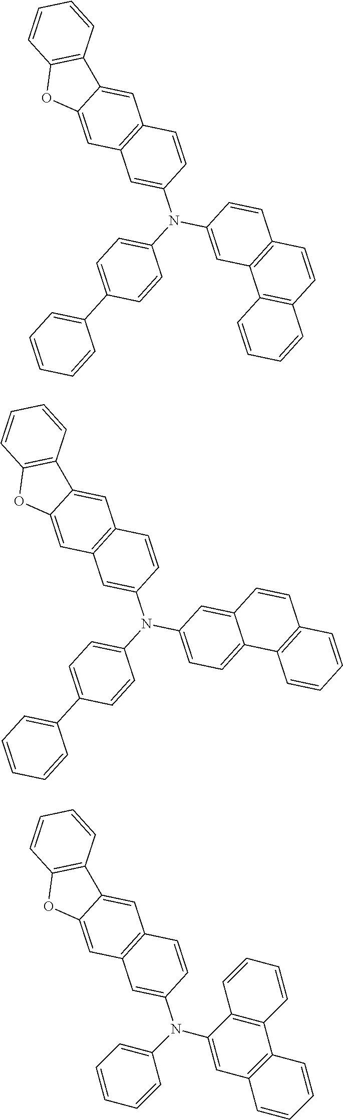 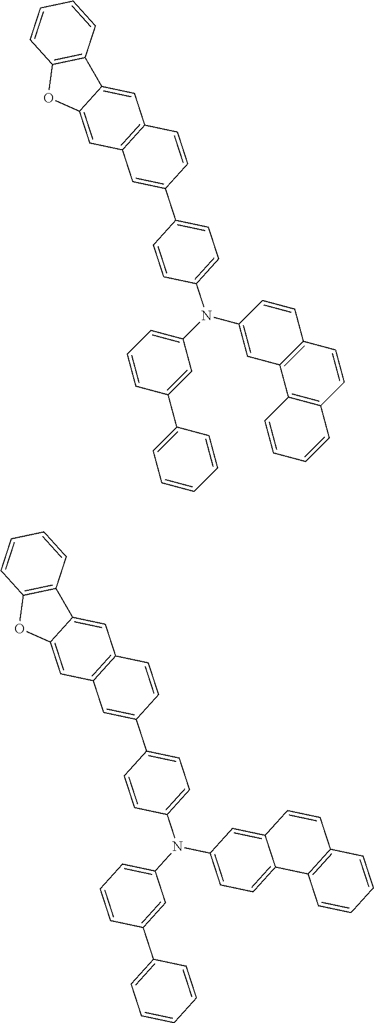 [SMILES: c1ccc(-c2cccc(N(c3ccc(-c4ccc5cc6c(cc5c4)oc4ccccc46)cc3)c3ccc4ccc5ccccc5c4c3)c2)cc1 ; c1ccc(-c2cccc(N(c3ccc(-c4ccc5cc6c(cc5c4)oc4ccccc46)cc3)c3ccc4c(ccc5ccccc54)c3)c2)cc1] 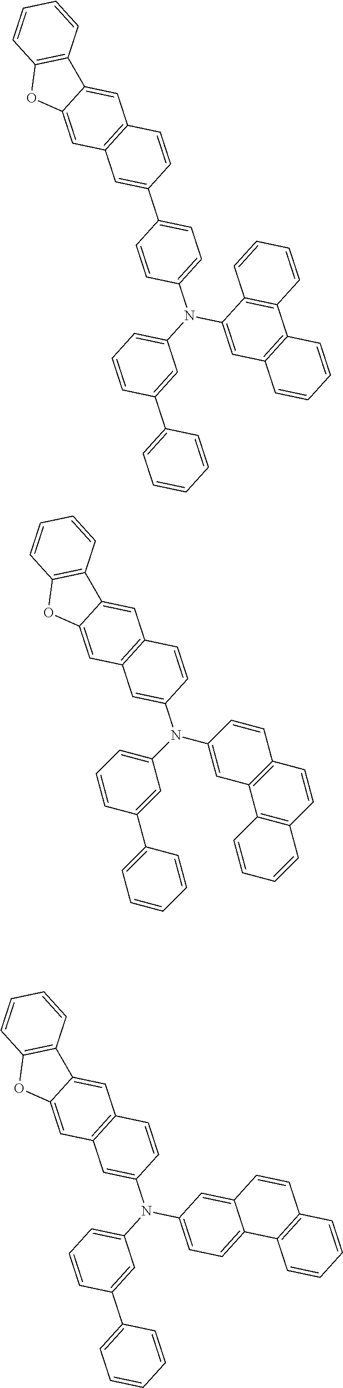 [SMILES: c1ccc(-c2cccc(N(c3ccc(-c4ccc5cc6c(cc5c4)oc4ccccc46)cc3)c3cc4ccccc4c4ccccc34)c2)cc1 ; c1ccc(-c2cccc(N(c3ccc4cc5c(cc4c3)oc3ccccc35)c3ccc4ccc5ccccc5c4c3)c2)cc1 ; c1ccc(-c2cccc(N(c3ccc4cc5c(cc4c3)oc3ccccc35)c3ccc4c(ccc5ccccc54)c3)c2)cc1] 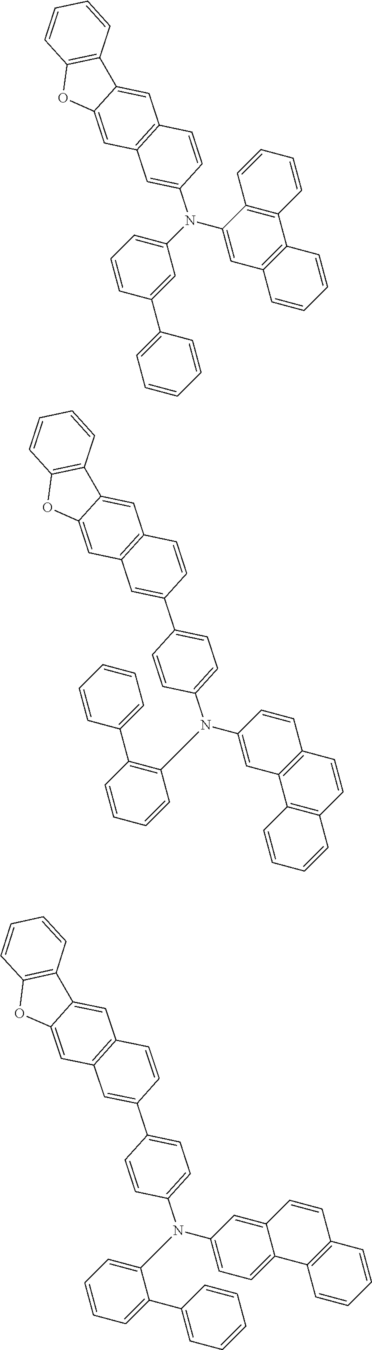 [SMILES: c1ccc(-c2cccc(N(c3ccc4cc5c(cc4c3)oc3ccccc35)c3cc4ccccc4c4ccccc34)c2)cc1 ; c1ccc(-c2ccccc2N(c2ccc(-c3ccc4cc5c(cc4c3)oc3ccccc35)cc2)c2ccc3ccc4ccccc4c3c2)cc1 ; c1ccc(-c2ccccc2N(c2ccc(-c3ccc4cc5c(cc4c3)oc3ccccc35)cc2)c2ccc3c(ccc4ccccc43)c2)cc1] 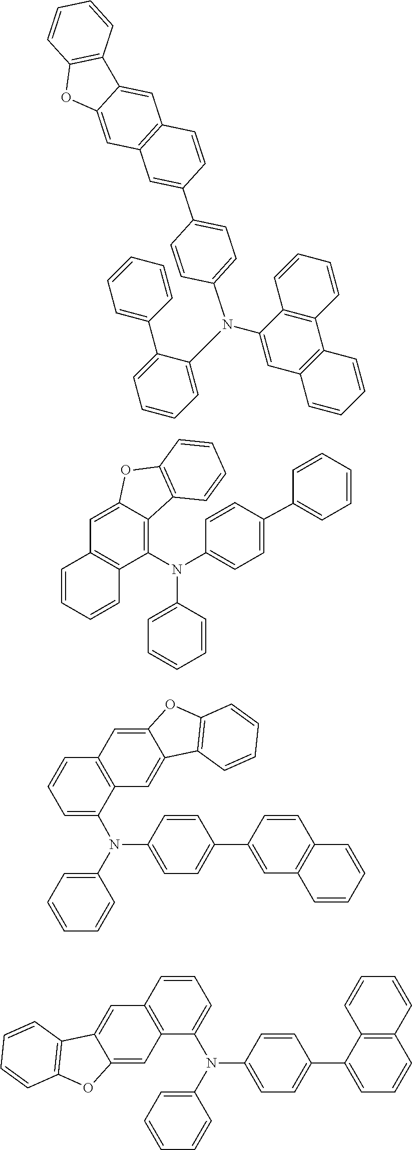 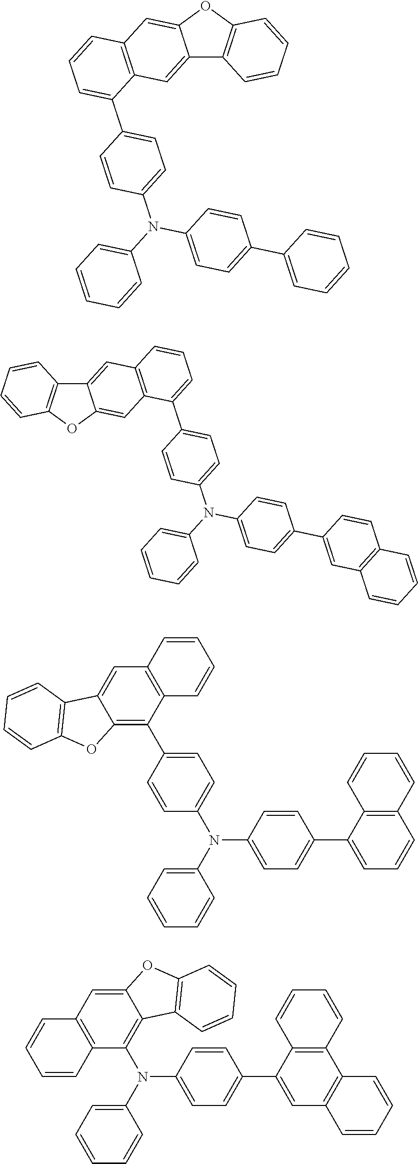 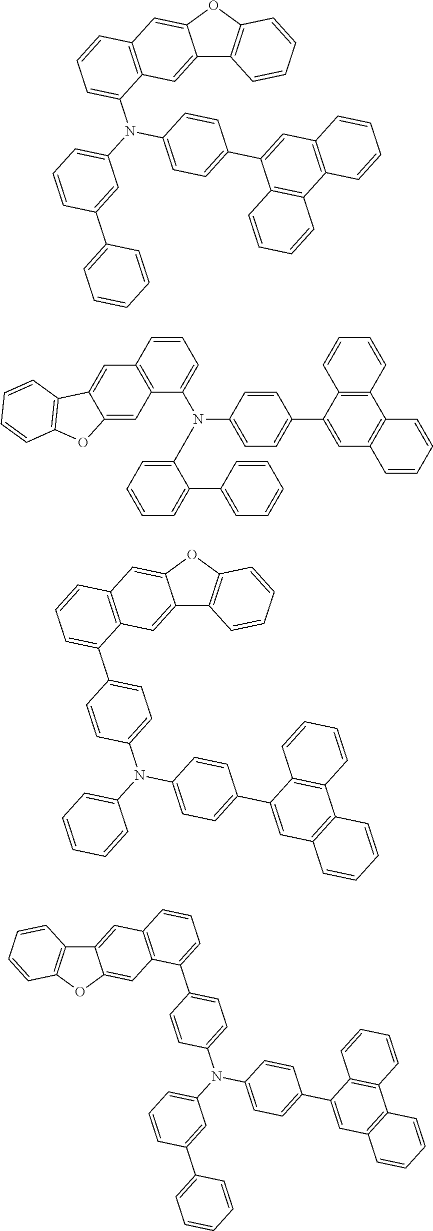 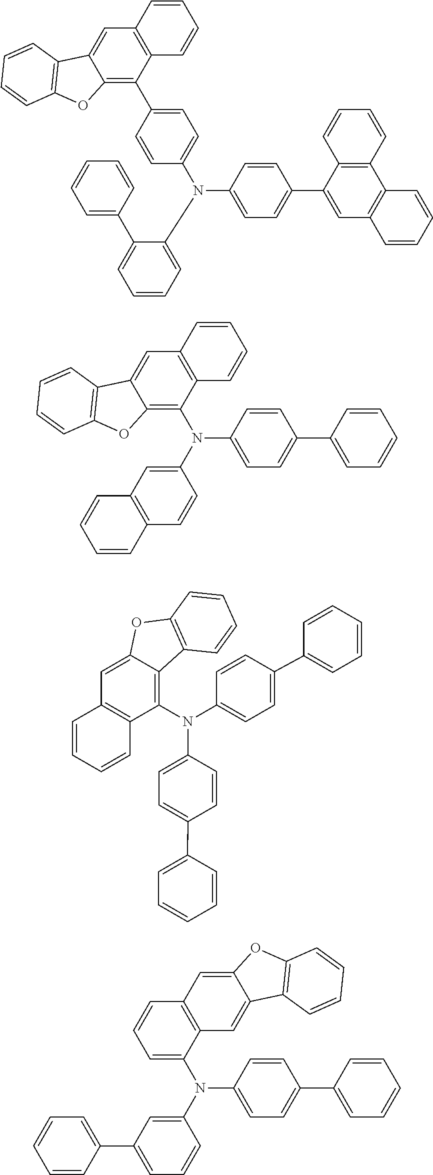 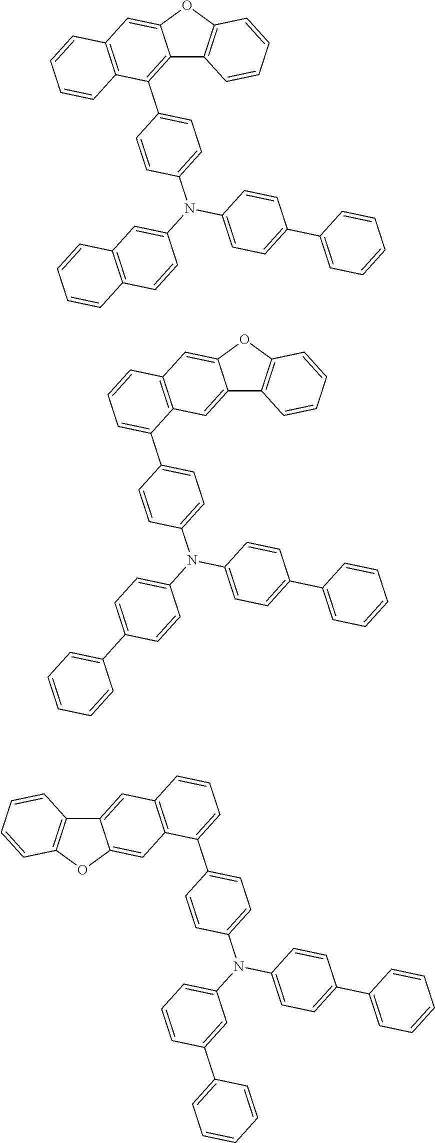 [SMILES: c1ccc(-c2ccc(N(c3ccc(-c4c5ccccc5cc5oc6ccccc6c45)cc3)c3ccc4ccccc4c3)cc2)cc1 ; c1ccc(-c2ccc(N(c3ccc(-c4ccccc4)cc3)c3ccc(-c4cccc5cc6oc7ccccc7c6cc45)cc3)cc2)cc1 ; c1ccc(-c2ccc(N(c3ccc(-c4cccc5cc6c(cc45)oc4ccccc46)cc3)c3cccc(-c4ccccc4)c3)cc2)cc1] 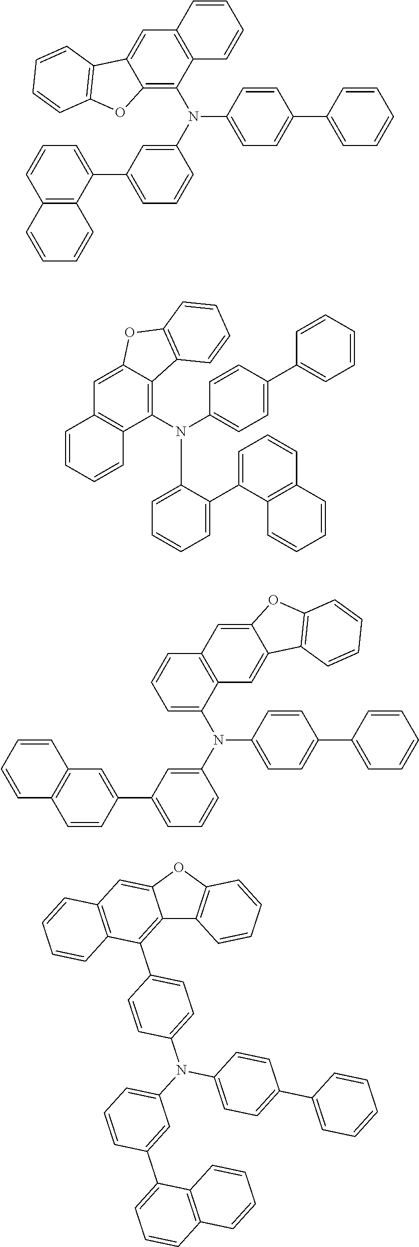 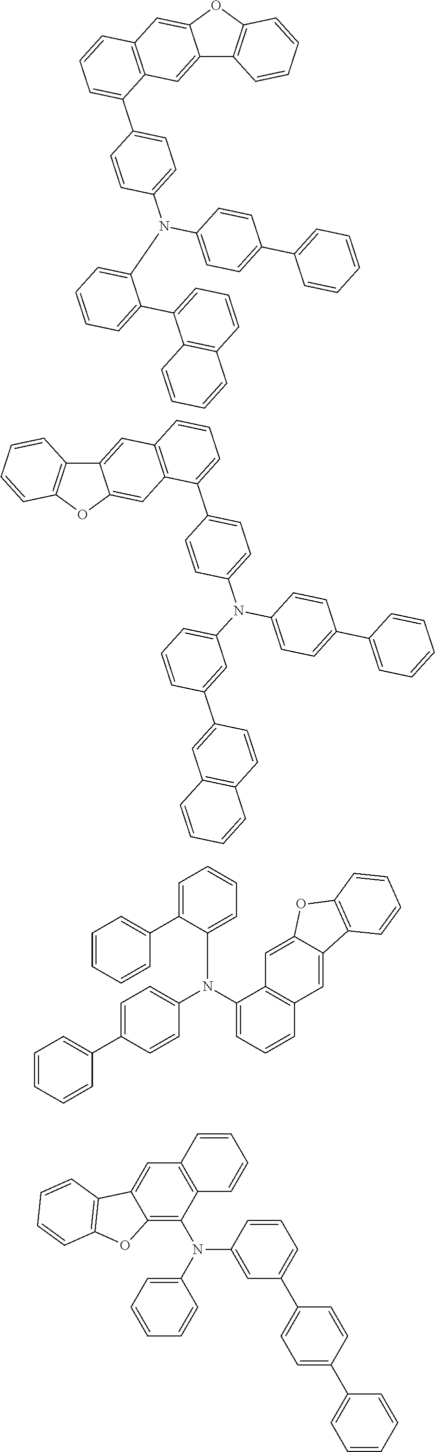 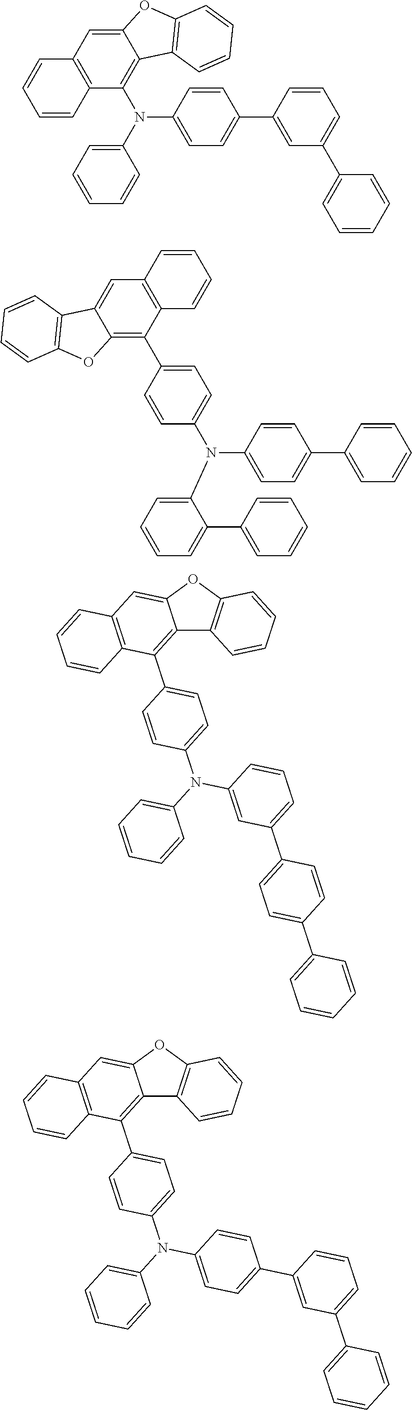 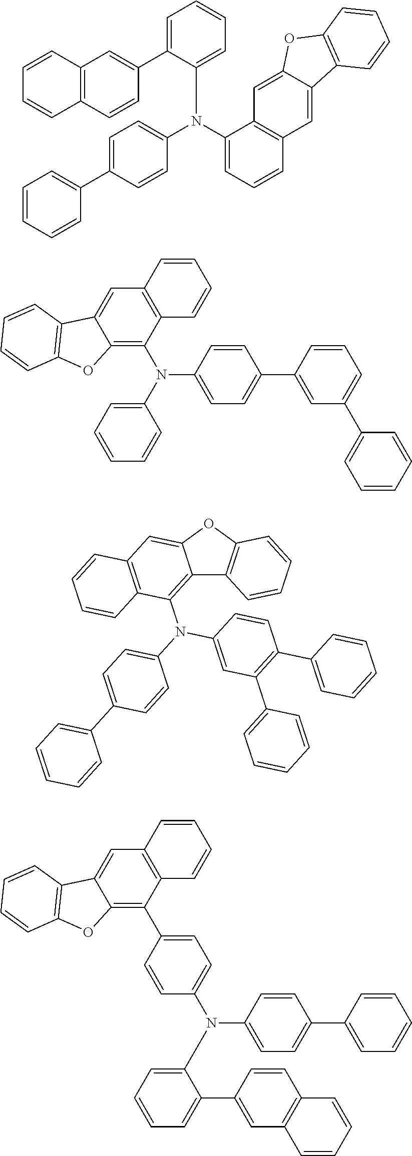 [SMILES: c1ccc(-c2ccc(N(c3ccccc3-c3ccc4ccccc4c3)c3cccc4cc5c(cc34)oc3ccccc35)cc2)cc1 ; c1ccc(-c2cccc(-c3ccc(N(c4ccccc4)c4c5ccccc5cc5c4oc4ccccc45)cc3)c2)cc1 ; c1ccc(-c2ccc(N(c3ccc(-c4ccccc4)c(-c4ccccc4)c3)c3c4ccccc4cc4oc5ccccc5c34)cc2)cc1 ; c1ccc(-c2ccc(N(c3ccc(-c4c5ccccc5cc5c4oc4ccccc45)cc3)c3ccccc3-c3ccc4ccccc4c3)cc2)cc1] 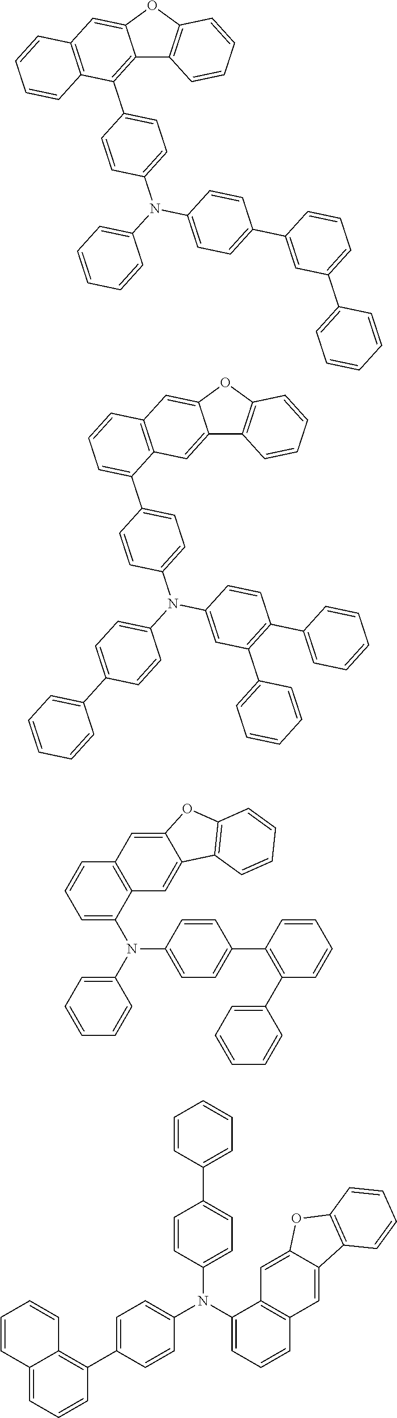 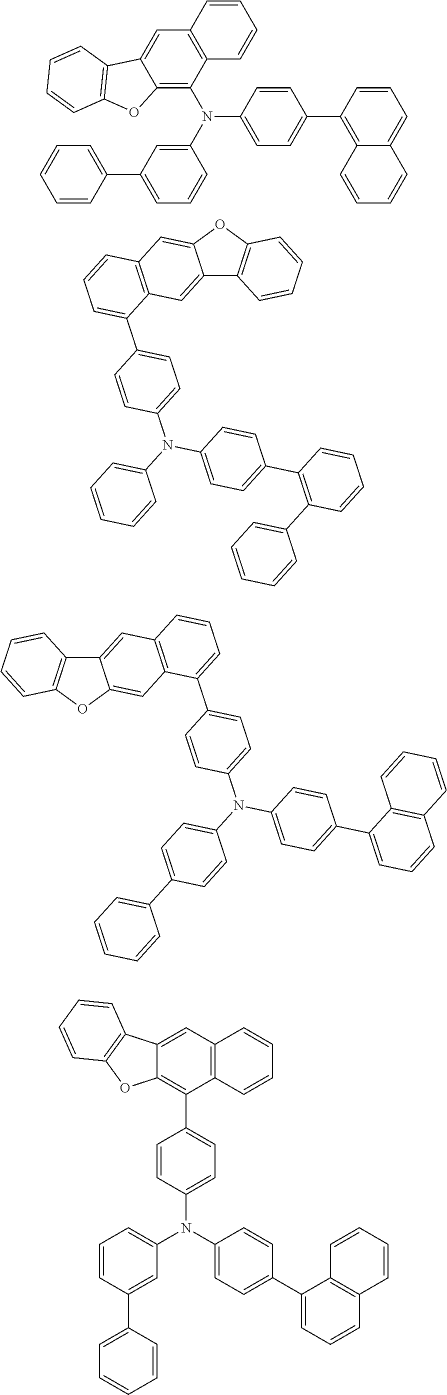 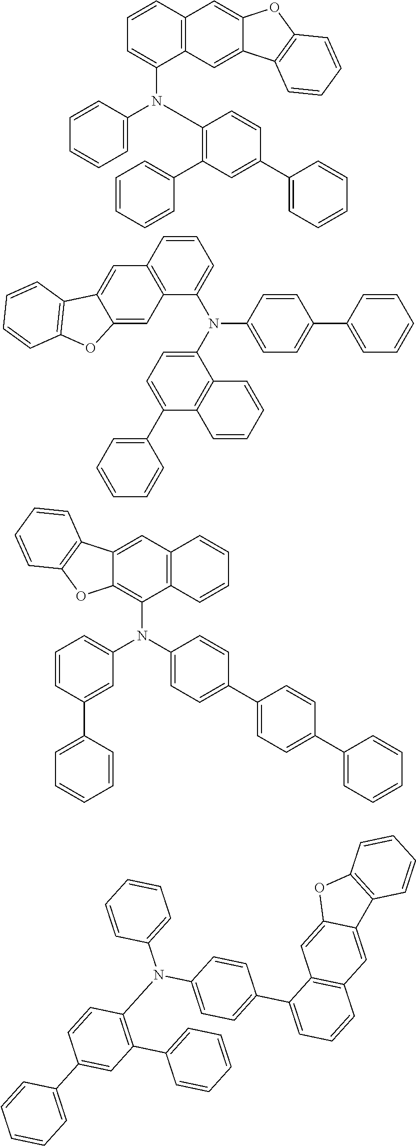 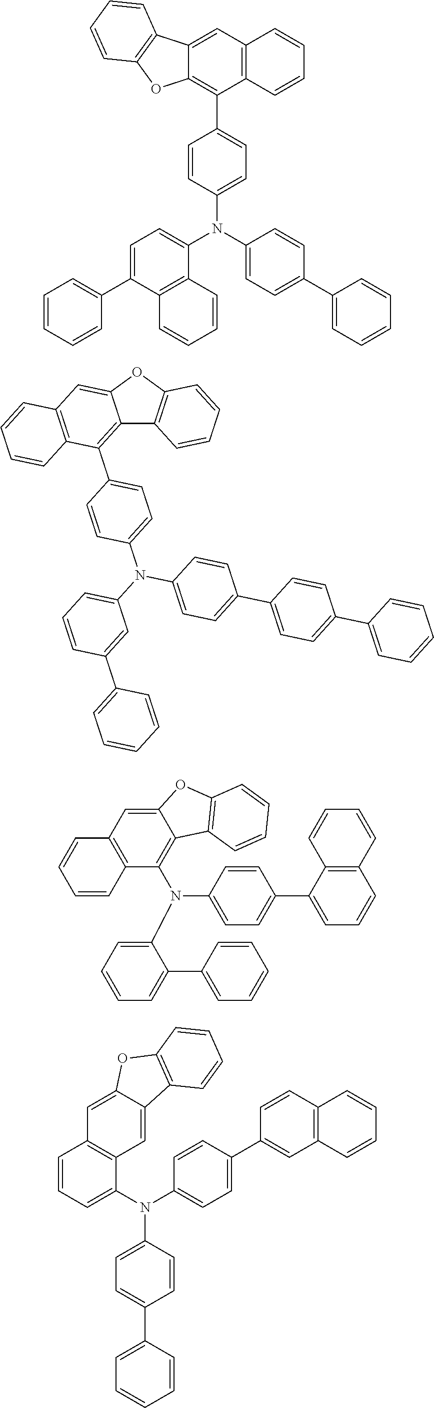 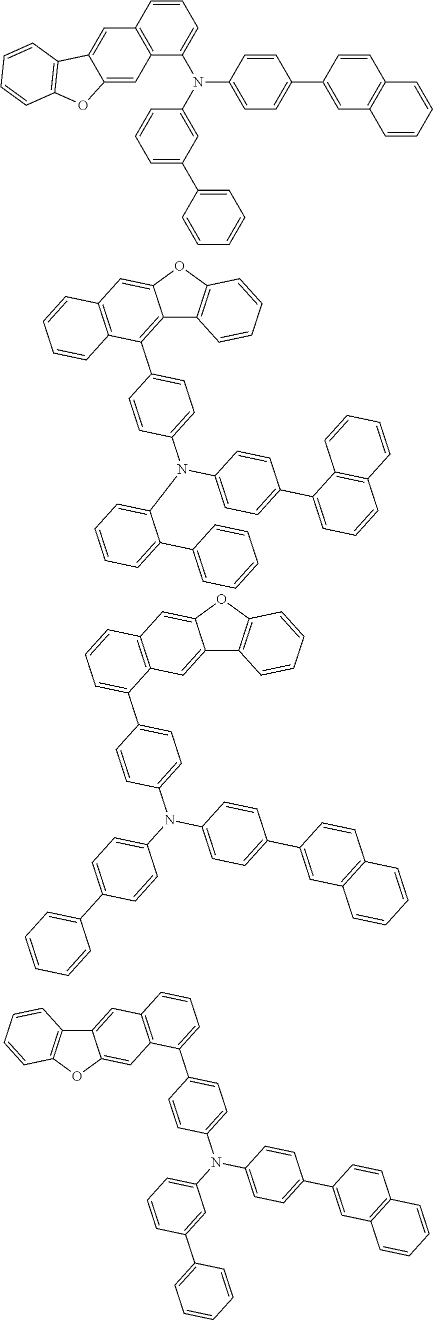 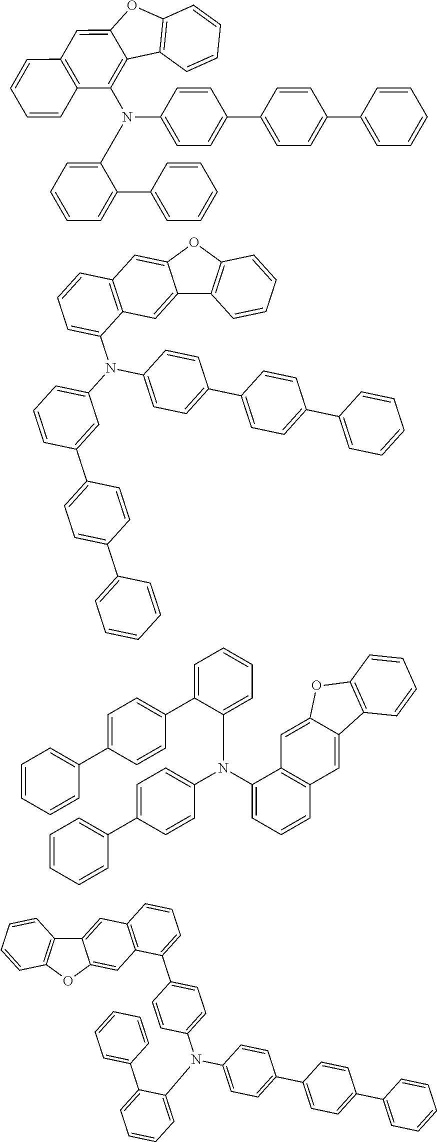 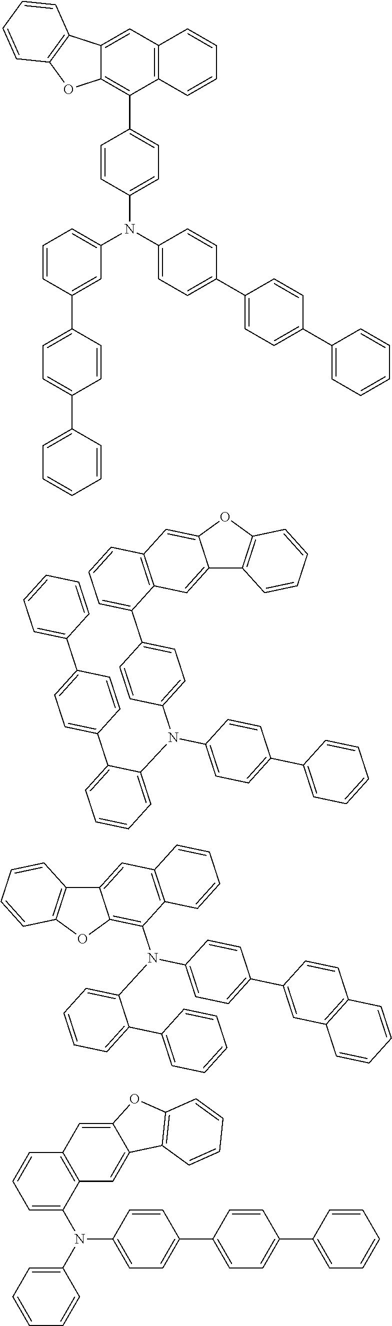 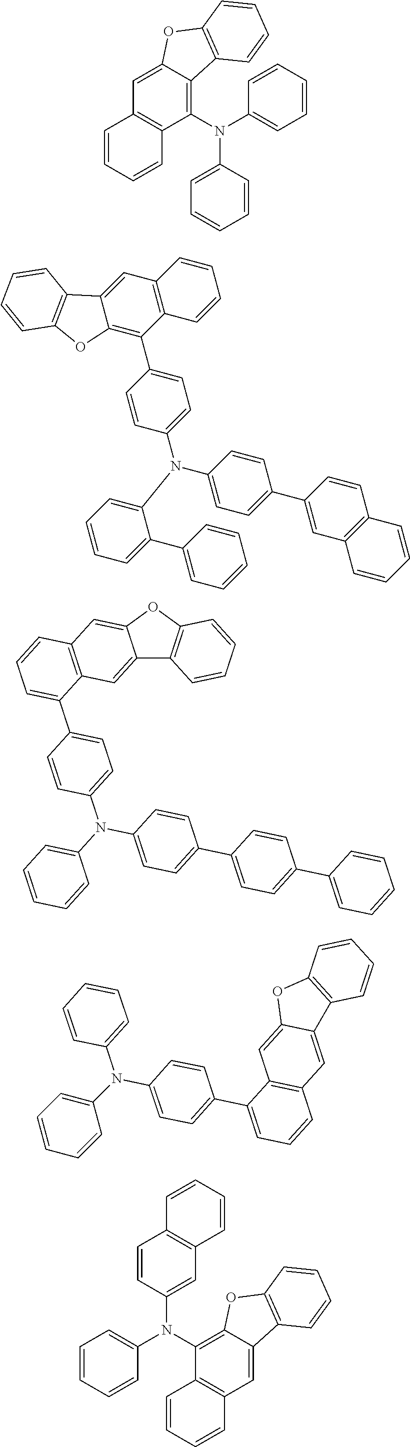 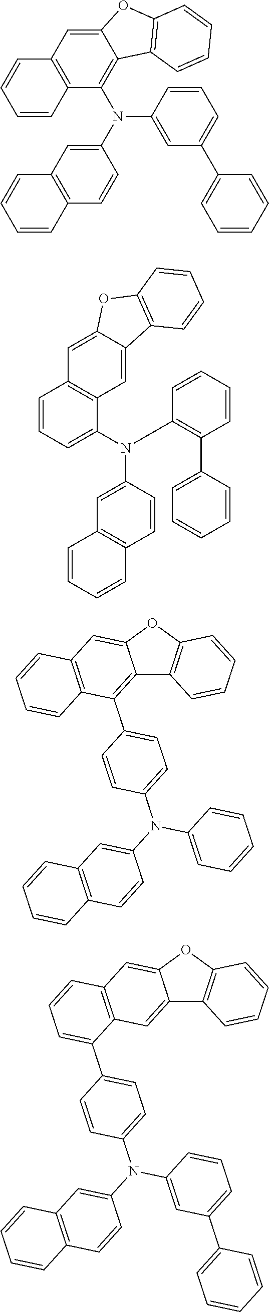 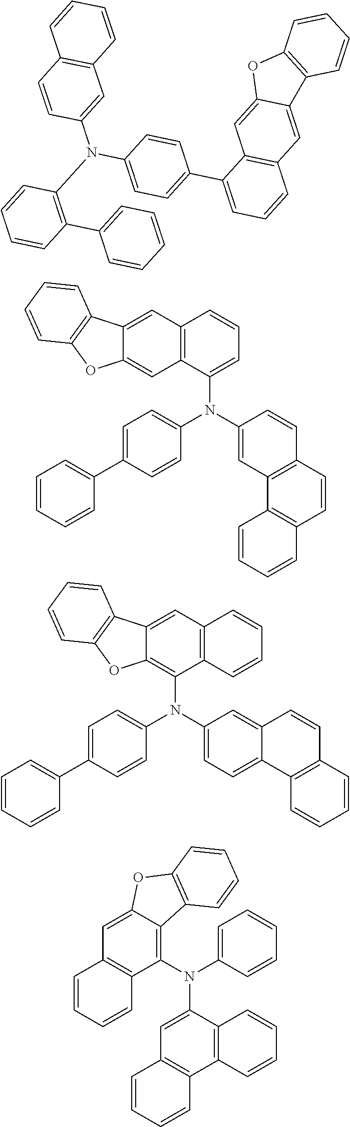 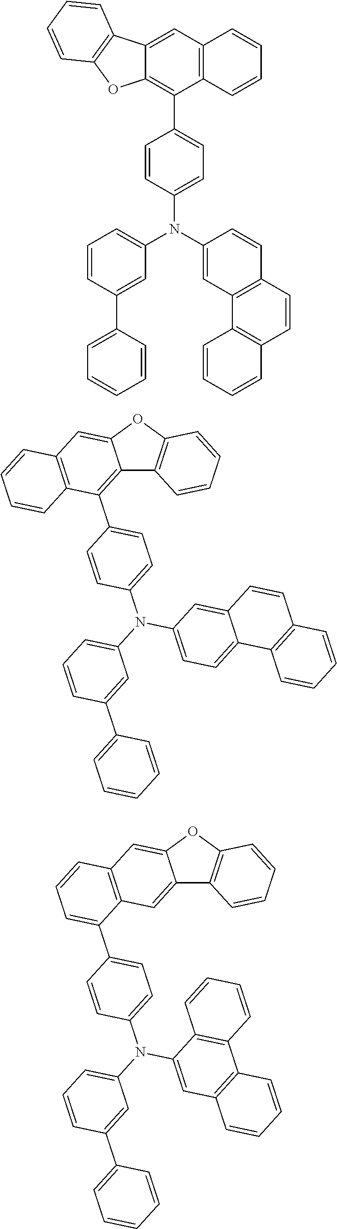 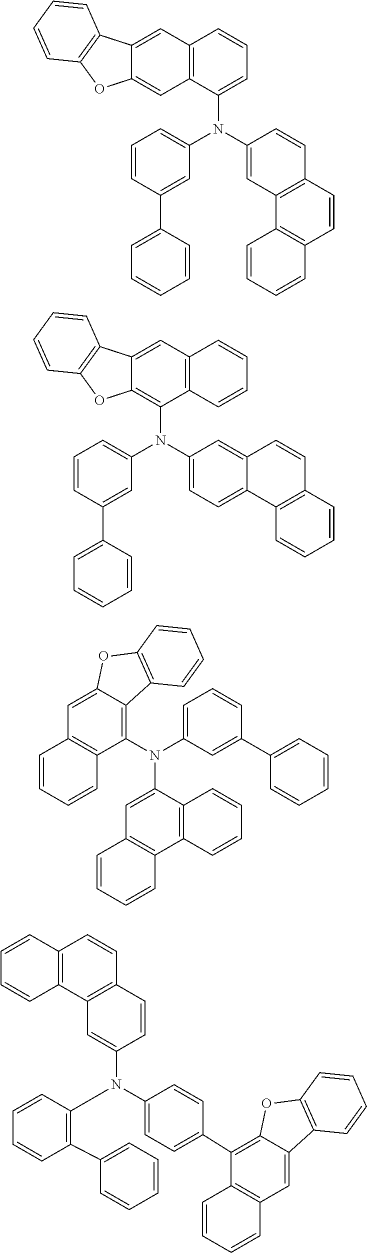 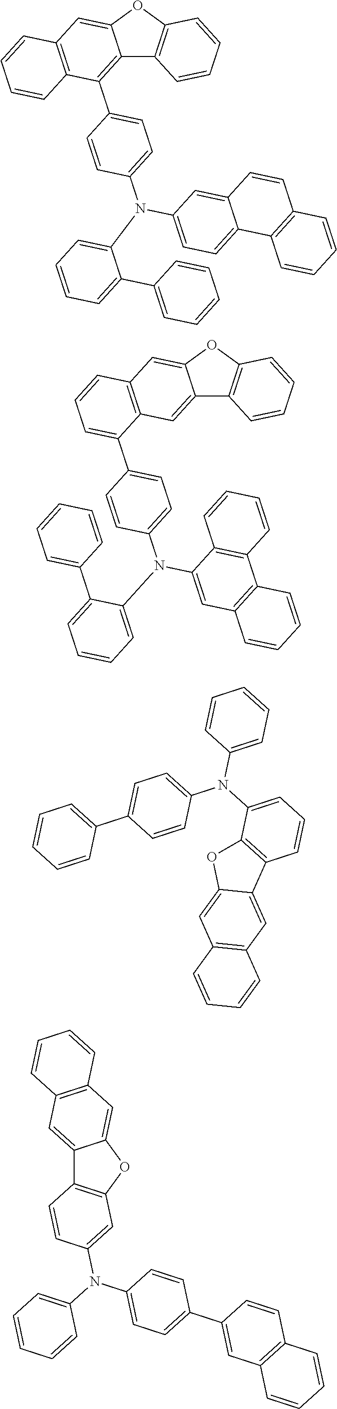 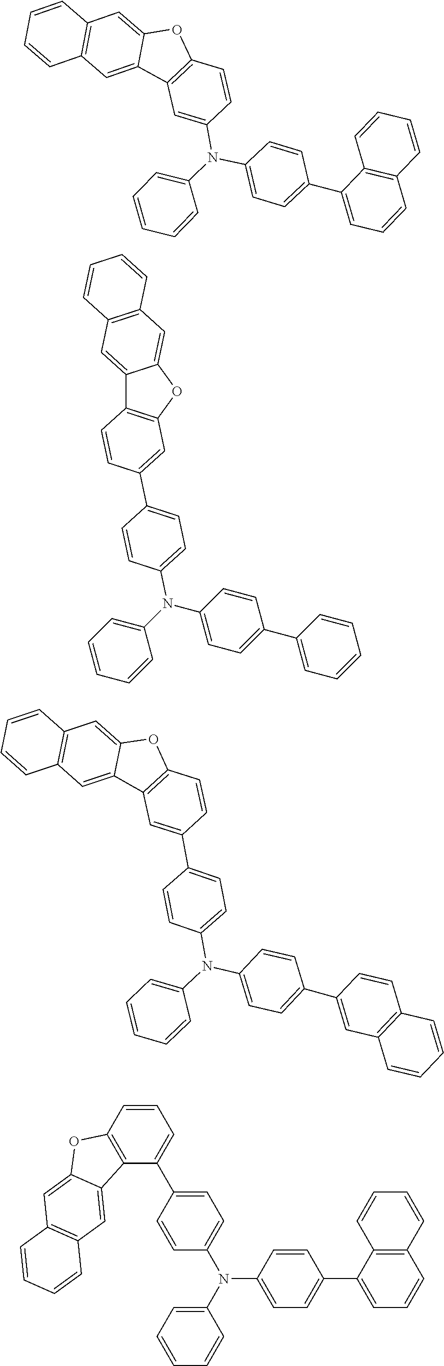 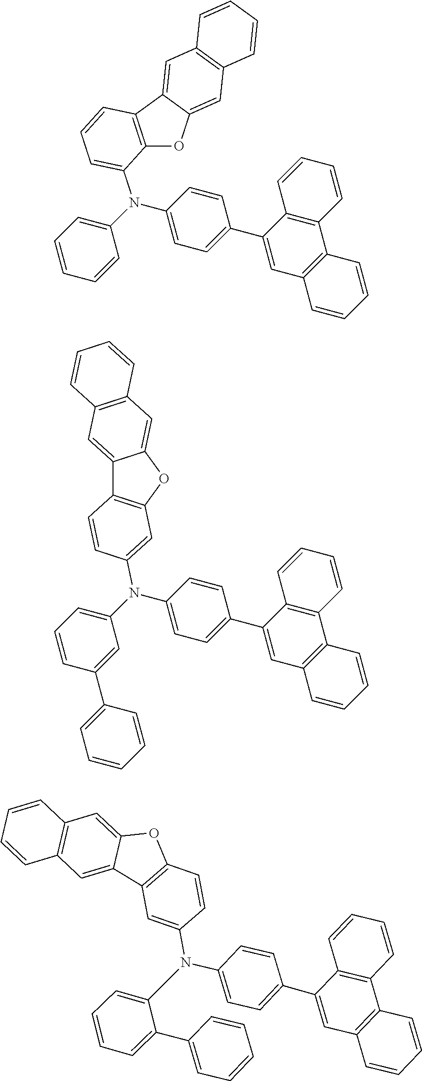 [SMILES: c1ccc(N(c2ccc(-c3cc4ccccc4c4ccccc34)cc2)c2cccc3c2oc2cc4ccccc4cc23)cc1 ; c1ccc(-c2cccc(N(c3ccc(-c4cc5ccccc5c5ccccc45)cc3)c3ccc4c(c3)oc3cc5ccccc5cc34)c2)cc1 ; c1ccc(-c2ccccc2N(c2ccc(-c3cc4ccccc4c4ccccc34)cc2)c2ccc3oc4cc5ccccc5cc4c3c2)cc1] 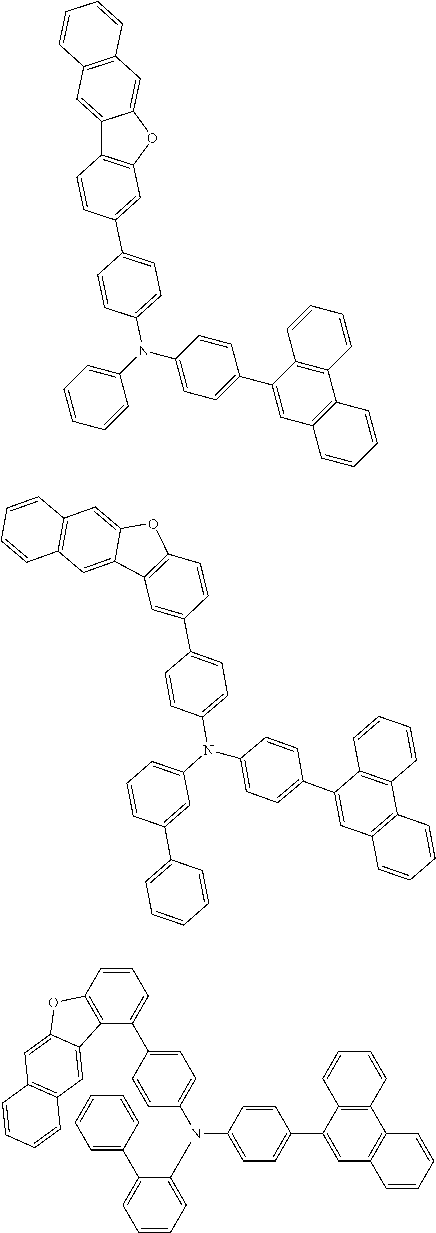 [SMILES: c1ccc(N(c2ccc(-c3ccc4c(c3)oc3cc5ccccc5cc34)cc2)c2ccc(-c3cc4ccccc4c4ccccc34)cc2)cc1 ; c1ccc(-c2cccc(N(c3ccc(-c4ccc5oc6cc7ccccc7cc6c5c4)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cccc4oc5cc6ccccc6cc5c34)cc2)cc1] 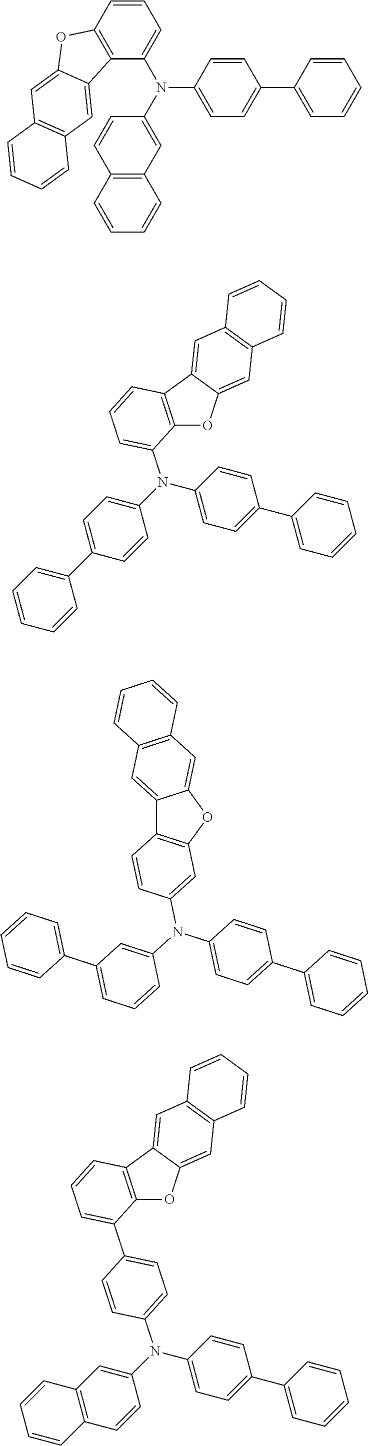 [SMILES: c1ccc(-c2ccc(N(c3ccc4ccccc4c3)c3cccc4oc5cc6ccccc6cc5c34)cc2)cc1 ; c1ccc(-c2ccc(N(c3ccc(-c4ccccc4)cc3)c3cccc4c3oc3cc5ccccc5cc34)cc2)cc1 ; c1ccc(-c2ccc(N(c3cccc(-c4ccccc4)c3)c3ccc4c(c3)oc3cc5ccccc5cc34)cc2)cc1 ; c1ccc(-c2ccc(N(c3ccc(-c4cccc5c4oc4cc6ccccc6cc45)cc3)c3ccc4ccccc4c3)cc2)cc1] 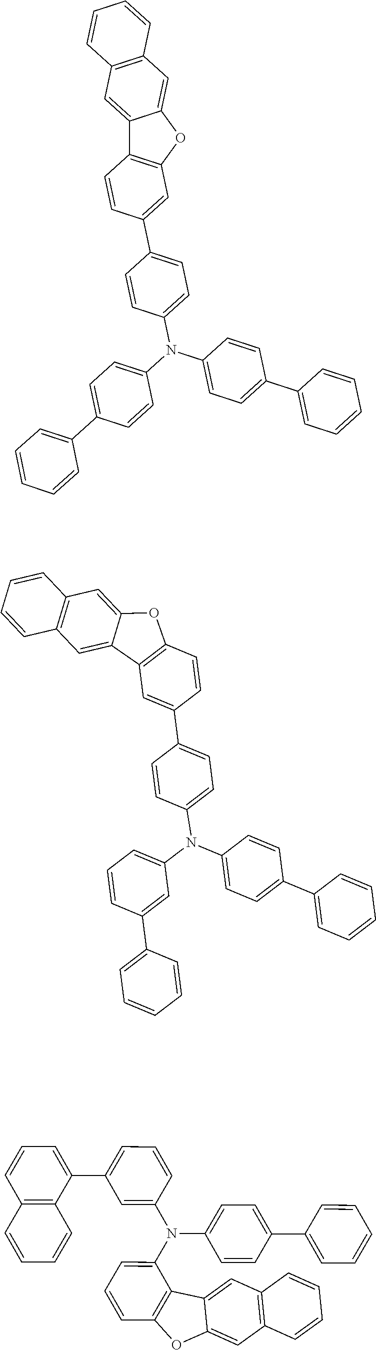 [SMILES: c1ccc(-c2ccc(N(c3ccc(-c4ccccc4)cc3)c3ccc(-c4ccc5c(c4)oc4cc6ccccc6cc45)cc3)cc2)cc1 ; c1ccc(-c2ccc(N(c3ccc(-c4ccc5oc6cc7ccccc7cc6c5c4)cc3)c3cccc(-c4ccccc4)c3)cc2)cc1 ; c1ccc(-c2ccc(N(c3cccc(-c4cccc5ccccc45)c3)c3cccc4oc5cc6ccccc6cc5c34)cc2)cc1] 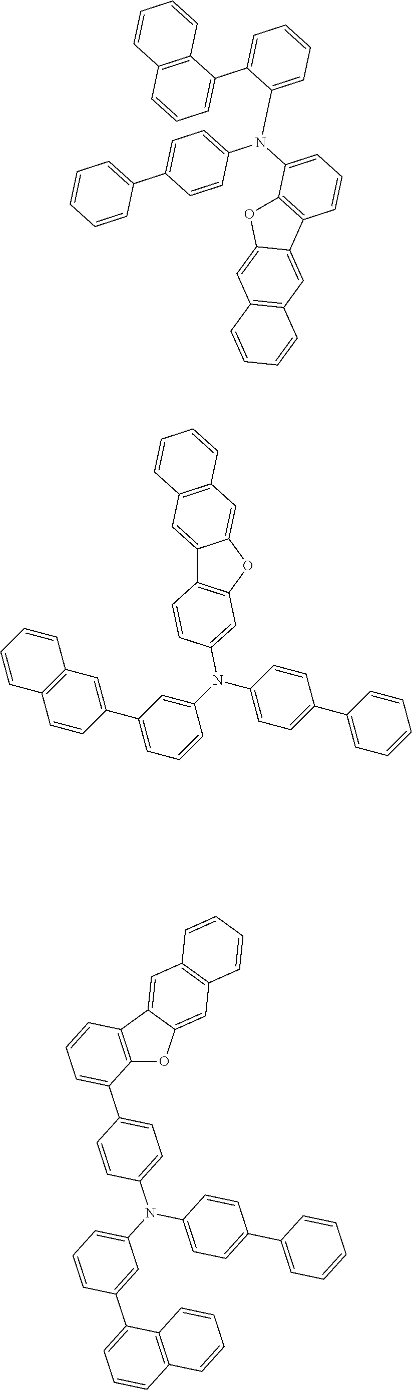 [SMILES: c1ccc(-c2ccc(N(c3ccccc3-c3cccc4ccccc34)c3cccc4c3oc3cc5ccccc5cc34)cc2)cc1 ; c1ccc(-c2ccc(N(c3cccc(-c4ccc5ccccc5c4)c3)c3ccc4c(c3)oc3cc5ccccc5cc34)cc2)cc1 ; c1ccc(-c2ccc(N(c3ccc(-c4cccc5c4oc4cc6ccccc6cc45)cc3)c3cccc(-c4cccc5ccccc45)c3)cc2)cc1] 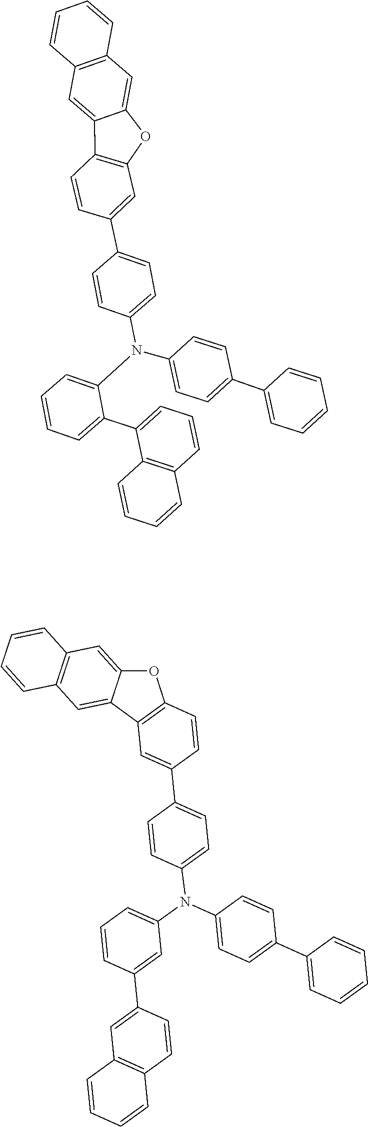 [SMILES: c1ccc(-c2ccc(N(c3ccc(-c4ccc5c(c4)oc4cc6ccccc6cc45)cc3)c3ccccc3-c3cccc4ccccc34)cc2)cc1 ; c1ccc(-c2ccc(N(c3ccc(-c4ccc5oc6cc7ccccc7cc6c5c4)cc3)c3cccc(-c4ccc5ccccc5c4)c3)cc2)cc1] 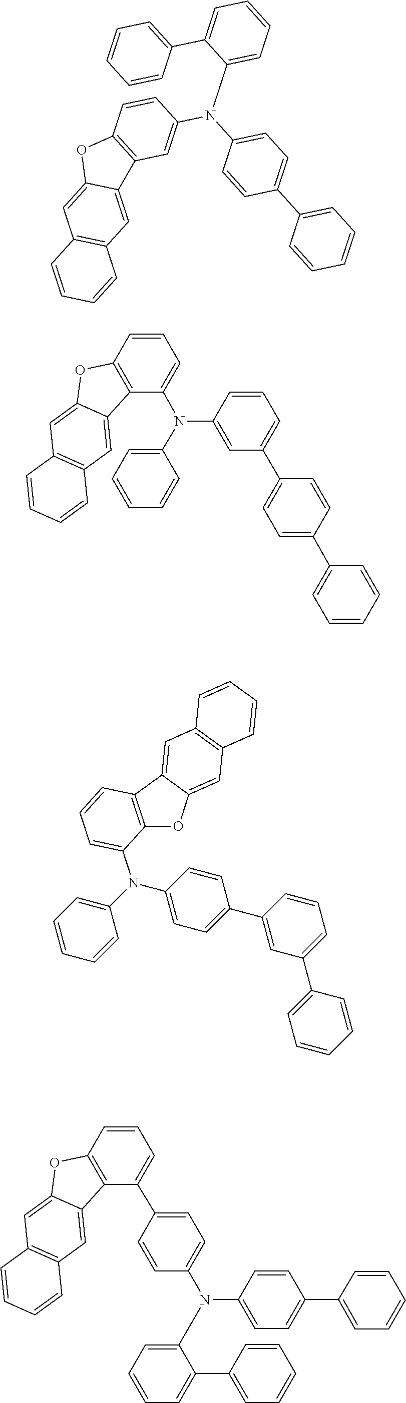 [SMILES: c1ccc(-c2ccc(N(c3ccc4oc5cc6ccccc6cc5c4c3)c3ccccc3-c3ccccc3)cc2)cc1 ; c1ccc(-c2ccc(-c3cccc(N(c4ccccc4)c4cccc5oc6cc7ccccc7cc6c45)c3)cc2)cc1 ; c1ccc(-c2cccc(-c3ccc(N(c4ccccc4)c4cccc5c4oc4cc6ccccc6cc45)cc3)c2)cc1 ; c1ccc(-c2ccc(N(c3ccc(-c4cccc5oc6cc7ccccc7cc6c45)cc3)c3ccccc3-c3ccccc3)cc2)cc1] 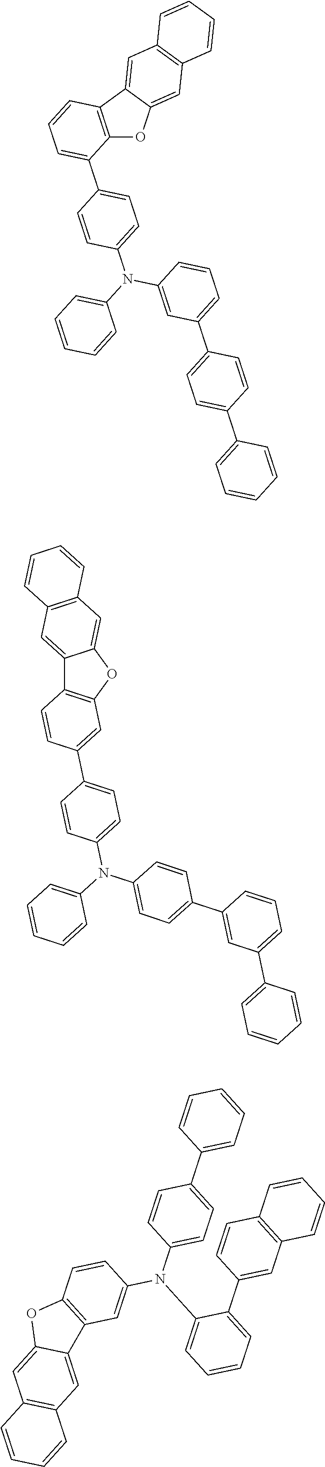 [SMILES: c1ccc(-c2ccc(-c3cccc(N(c4ccccc4)c4ccc(-c5cccc6c5oc5cc7ccccc7cc56)cc4)c3)cc2)cc1 ; c1ccc(-c2cccc(-c3ccc(N(c4ccccc4)c4ccc(-c5ccc6c(c5)oc5cc7ccccc7cc56)cc4)cc3)c2)cc1 ; c1ccc(-c2ccc(N(c3ccc4oc5cc6ccccc6cc5c4c3)c3ccccc3-c3ccc4ccccc4c3)cc2)cc1] 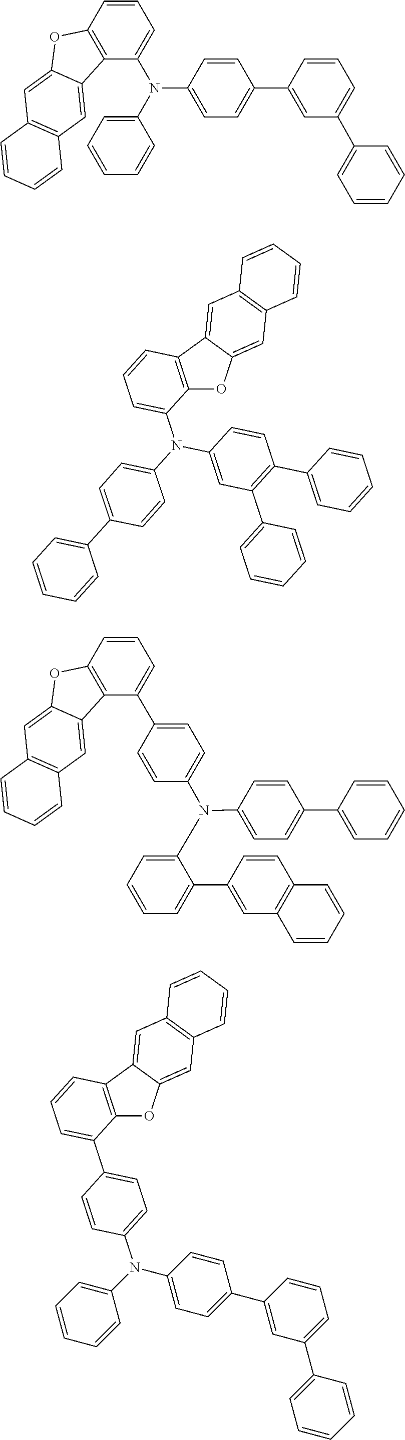 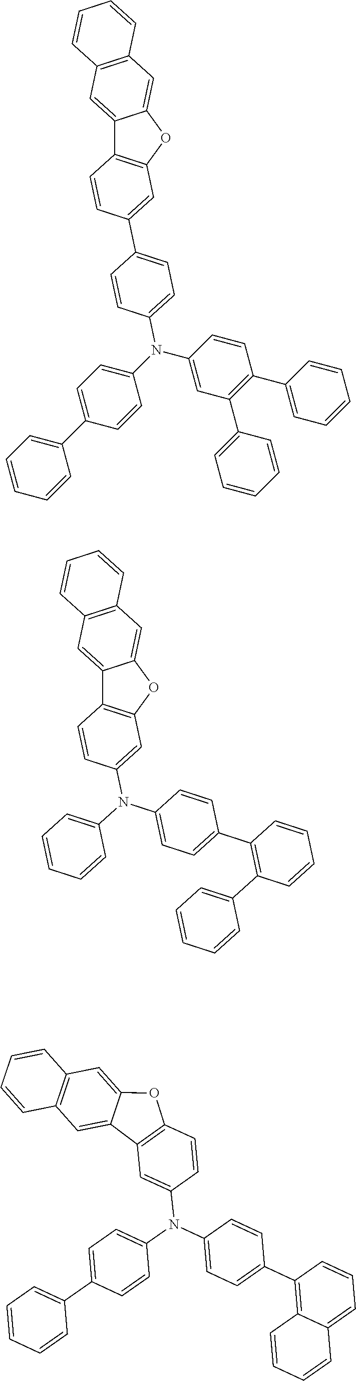 [SMILES: c1ccc(-c2ccc(N(c3ccc(-c4ccc5c(c4)oc4cc6ccccc6cc45)cc3)c3ccc(-c4ccccc4)c(-c4ccccc4)c3)cc2)cc1 ; c1ccc(-c2ccccc2-c2ccc(N(c3ccccc3)c3ccc4c(c3)oc3cc5ccccc5cc34)cc2)cc1 ; c1ccc(-c2ccc(N(c3ccc(-c4cccc5ccccc45)cc3)c3ccc4oc5cc6ccccc6cc5c4c3)cc2)cc1] 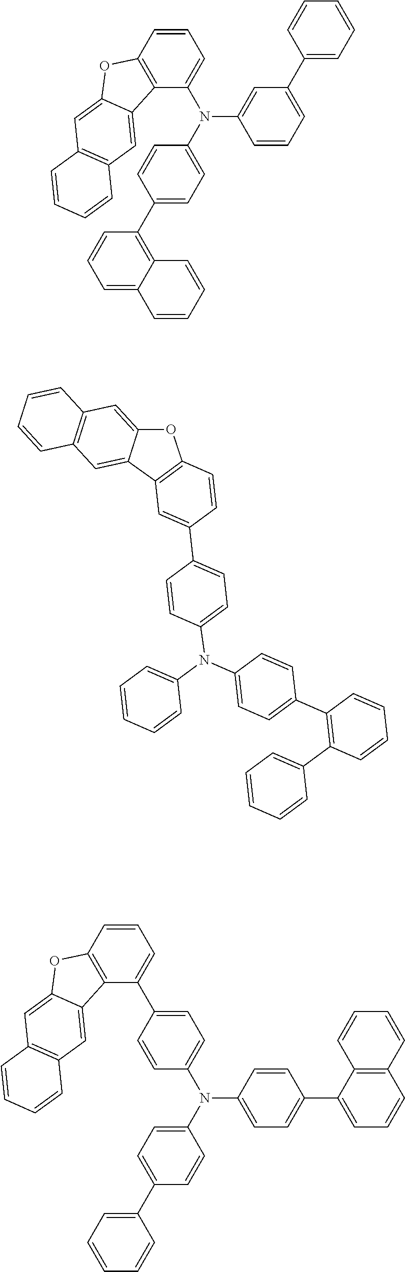 [SMILES: c1ccc(-c2cccc(N(c3ccc(-c4cccc5ccccc45)cc3)c3cccc4oc5cc6ccccc6cc5c34)c2)cc1 ; c1ccc(-c2ccccc2-c2ccc(N(c3ccccc3)c3ccc(-c4ccc5oc6cc7ccccc7cc6c5c4)cc3)cc2)cc1 ; c1ccc(-c2ccc(N(c3ccc(-c4cccc5ccccc45)cc3)c3ccc(-c4cccc5oc6cc7ccccc7cc6c45)cc3)cc2)cc1] 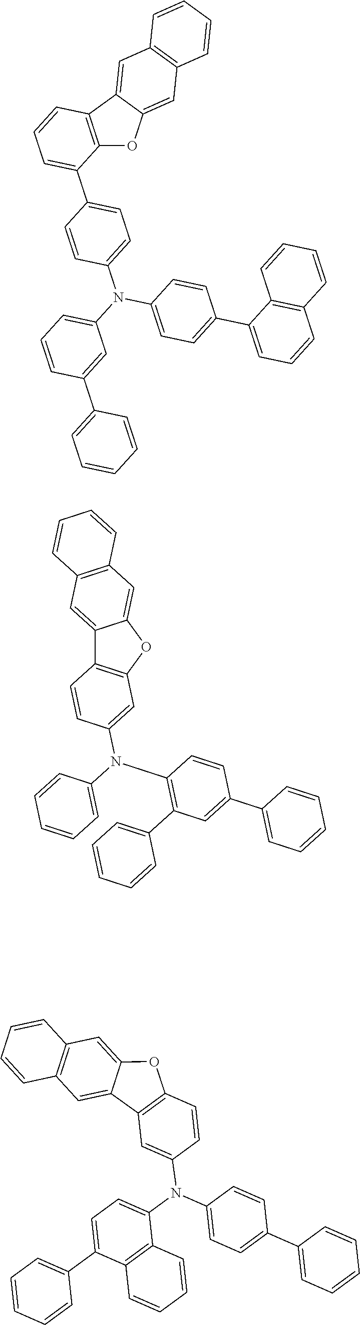 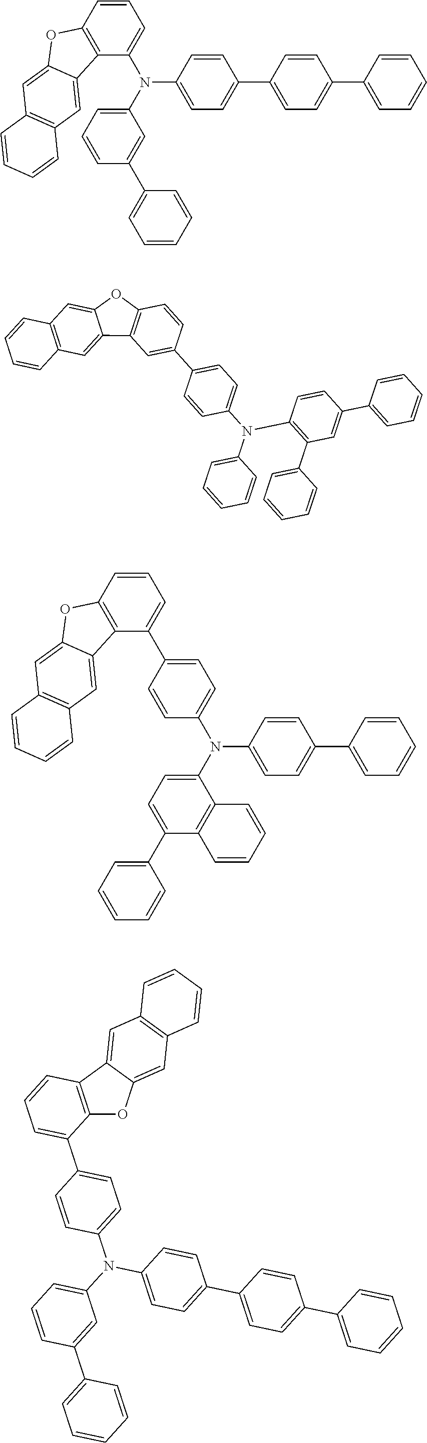 [SMILES: c1ccc(-c2ccc(-c3ccc(N(c4cccc(-c5ccccc5)c4)c4cccc5oc6cc7ccccc7cc6c45)cc3)cc2)cc1 ; c1ccc(-c2ccc(N(c3ccccc3)c3ccc(-c4ccc5oc6cc7ccccc7cc6c5c4)cc3)c(-c3ccccc3)c2)cc1 ; c1ccc(-c2ccc(N(c3ccc(-c4cccc5oc6cc7ccccc7cc6c45)cc3)c3ccc(-c4ccccc4)c4ccccc34)cc2)cc1 ; c1ccc(-c2ccc(-c3ccc(N(c4ccc(-c5cccc6c5oc5cc7ccccc7cc56)cc4)c4cccc(-c5ccccc5)c4)cc3)cc2)cc1] 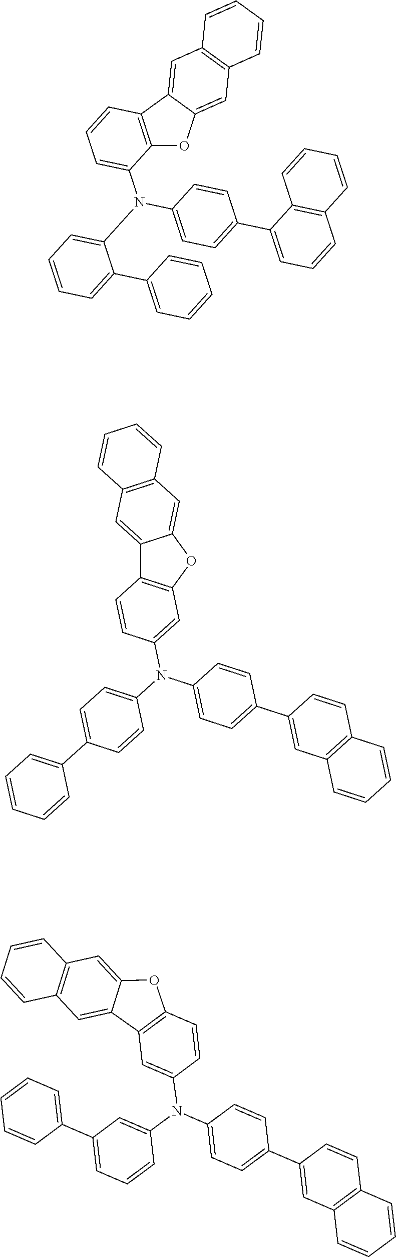 [SMILES: c1ccc(-c2ccccc2N(c2ccc(-c3cccc4ccccc34)cc2)c2cccc3c2oc2cc4ccccc4cc23)cc1 ; c1ccc(-c2ccc(N(c3ccc(-c4ccc5ccccc5c4)cc3)c3ccc4c(c3)oc3cc5ccccc5cc34)cc2)cc1 ; c1ccc(-c2cccc(N(c3ccc(-c4ccc5ccccc5c4)cc3)c3ccc4oc5cc6ccccc6cc5c4c3)c2)cc1] 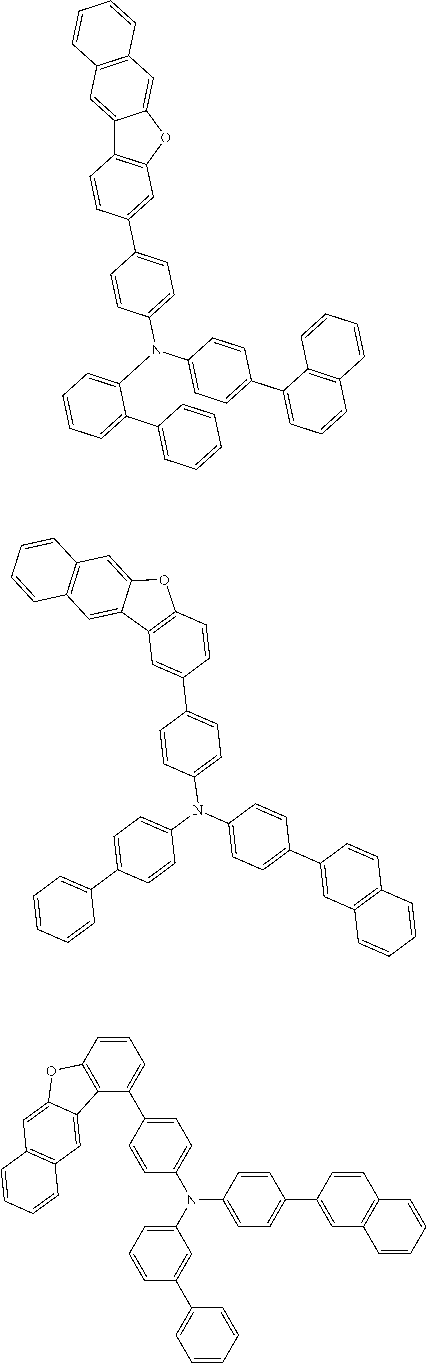 [SMILES: c1ccc(-c2ccccc2N(c2ccc(-c3ccc4c(c3)oc3cc5ccccc5cc34)cc2)c2ccc(-c3cccc4ccccc34)cc2)cc1 ; c1ccc(-c2ccc(N(c3ccc(-c4ccc5ccccc5c4)cc3)c3ccc(-c4ccc5oc6cc7ccccc7cc6c5c4)cc3)cc2)cc1 ; c1ccc(-c2cccc(N(c3ccc(-c4ccc5ccccc5c4)cc3)c3ccc(-c4cccc5oc6cc7ccccc7cc6c45)cc3)c2)cc1] 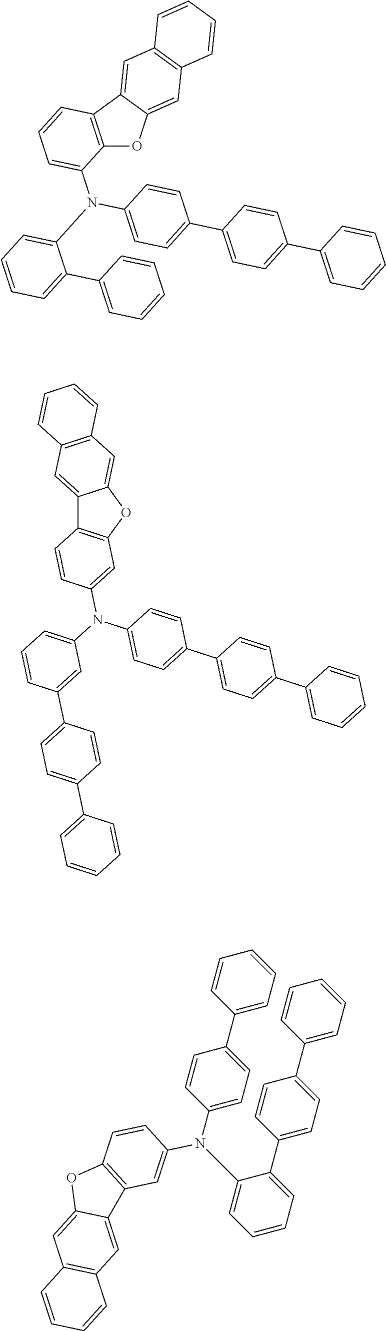 [SMILES: c1ccc(-c2ccc(-c3ccc(N(c4ccccc4-c4ccccc4)c4cccc5c4oc4cc6ccccc6cc45)cc3)cc2)cc1 ; c1ccc(-c2ccc(-c3ccc(N(c4cccc(-c5ccc(-c6ccccc6)cc5)c4)c4ccc5c(c4)oc4cc6ccccc6cc45)cc3)cc2)cc1 ; c1ccc(-c2ccc(-c3ccccc3N(c3ccc(-c4ccccc4)cc3)c3ccc4oc5cc6ccccc6cc5c4c3)cc2)cc1] 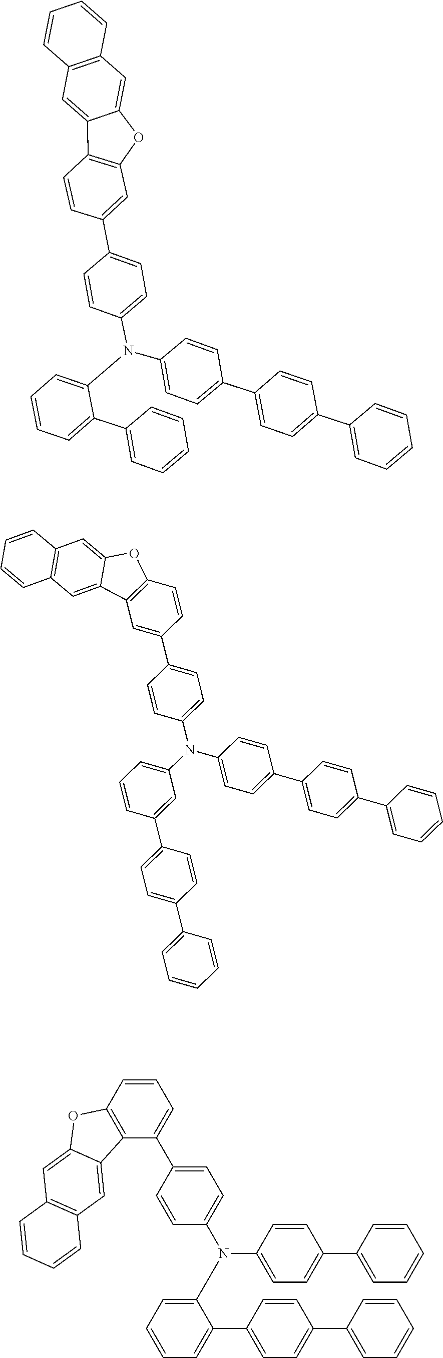 [SMILES: c1ccc(-c2ccc(-c3ccc(N(c4ccc(-c5ccc6c(c5)oc5cc7ccccc7cc56)cc4)c4ccccc4-c4ccccc4)cc3)cc2)cc1 ; c1ccc(-c2ccc(-c3ccc(N(c4ccc(-c5ccc6oc7cc8ccccc8cc7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5oc6cc7ccccc7cc6c45)cc3)cc2)cc1] 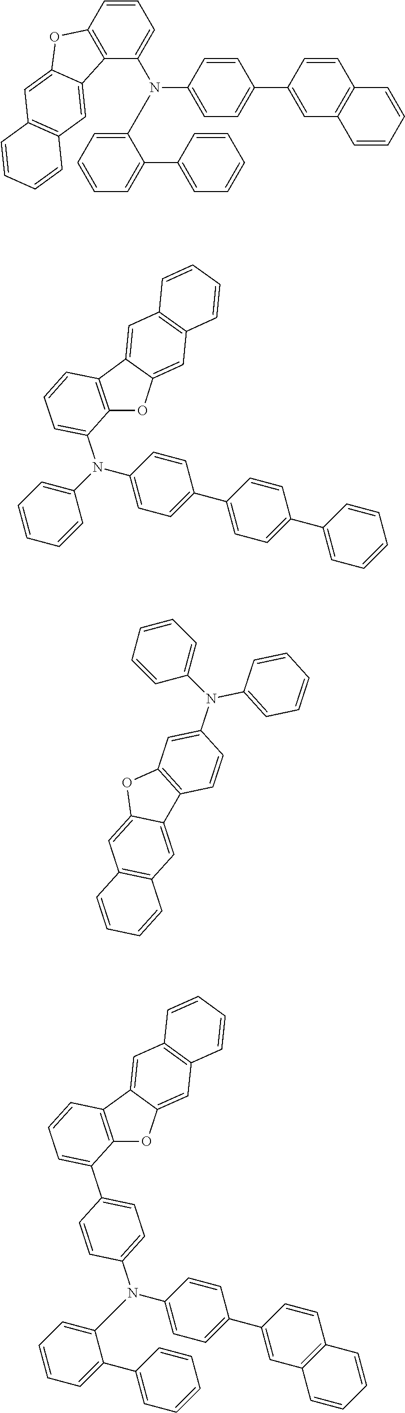 [SMILES: c1ccc(-c2ccccc2N(c2ccc(-c3ccc4ccccc4c3)cc2)c2cccc3oc4cc5ccccc5cc4c23)cc1 ; c1ccc(-c2ccc(-c3ccc(N(c4ccccc4)c4cccc5c4oc4cc6ccccc6cc45)cc3)cc2)cc1 ; c1ccc(N(c2ccccc2)c2ccc3c(c2)oc2cc4ccccc4cc23)cc1 ; c1ccc(-c2ccccc2N(c2ccc(-c3ccc4ccccc4c3)cc2)c2ccc(-c3cccc4c3oc3cc5ccccc5cc34)cc2)cc1] 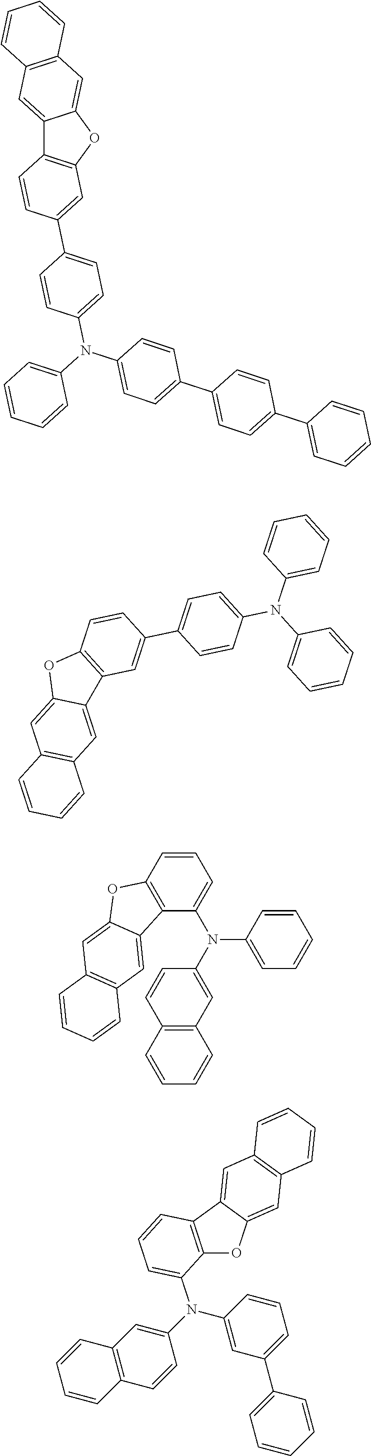 [SMILES: c1ccc(-c2ccc(-c3ccc(N(c4ccccc4)c4ccc(-c5ccc6c(c5)oc5cc7ccccc7cc56)cc4)cc3)cc2)cc1 ; c1ccc(N(c2ccccc2)c2ccc(-c3ccc4oc5cc6ccccc6cc5c4c3)cc2)cc1 ; c1ccc(N(c2ccc3ccccc3c2)c2cccc3oc4cc5ccccc5cc4c23)cc1 ; c1ccc(-c2cccc(N(c3ccc4ccccc4c3)c3cccc4c3oc3cc5ccccc5cc34)c2)cc1] 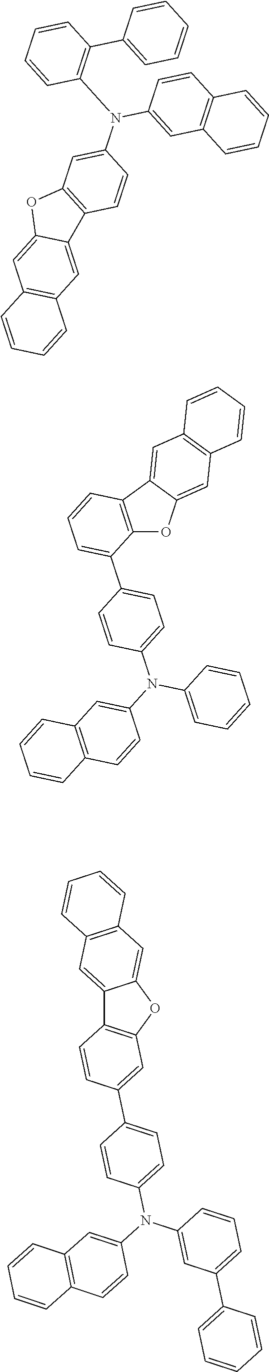 [SMILES: c1ccc(-c2ccccc2N(c2ccc3ccccc3c2)c2ccc3c(c2)oc2cc4ccccc4cc23)cc1 ; c1ccc(N(c2ccc(-c3cccc4c3oc3cc5ccccc5cc34)cc2)c2ccc3ccccc3c2)cc1 ; c1ccc(-c2cccc(N(c3ccc(-c4ccc5c(c4)oc4cc6ccccc6cc45)cc3)c3ccc4ccccc4c3)c2)cc1] 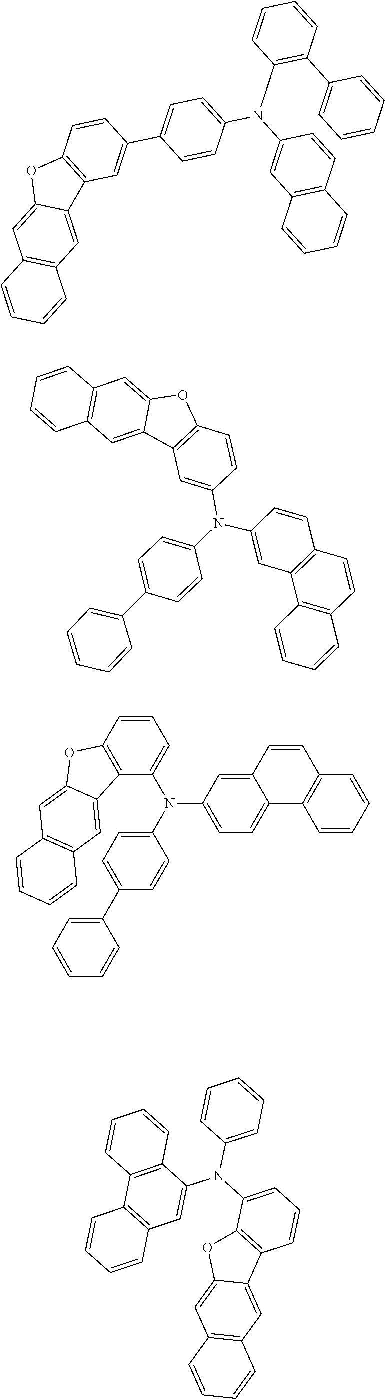 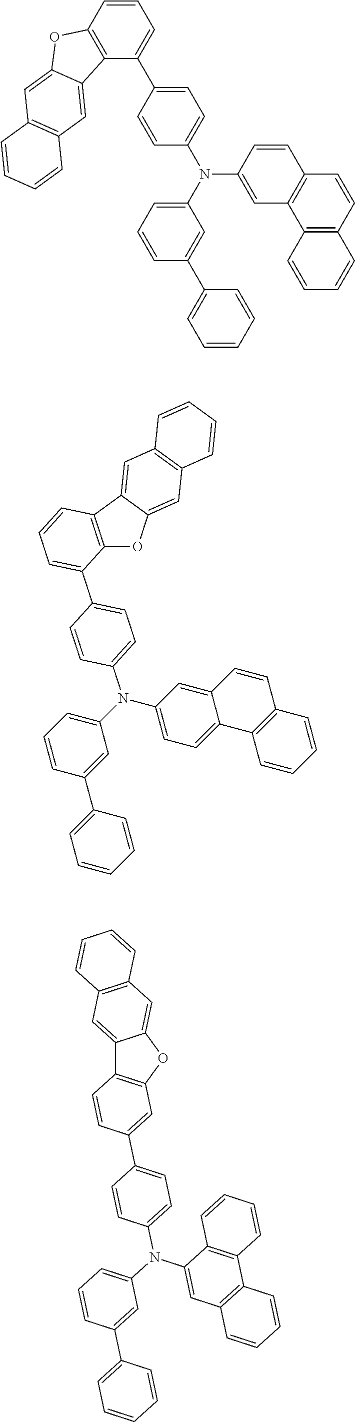 [SMILES: c1ccc(-c2cccc(N(c3ccc(-c4cccc5oc6cc7ccccc7cc6c45)cc3)c3ccc4ccc5ccccc5c4c3)c2)cc1 ; c1ccc(-c2cccc(N(c3ccc(-c4cccc5c4oc4cc6ccccc6cc45)cc3)c3ccc4c(ccc5ccccc54)c3)c2)cc1 ; c1ccc(-c2cccc(N(c3ccc(-c4ccc5c(c4)oc4cc6ccccc6cc45)cc3)c3cc4ccccc4c4ccccc34)c2)cc1] 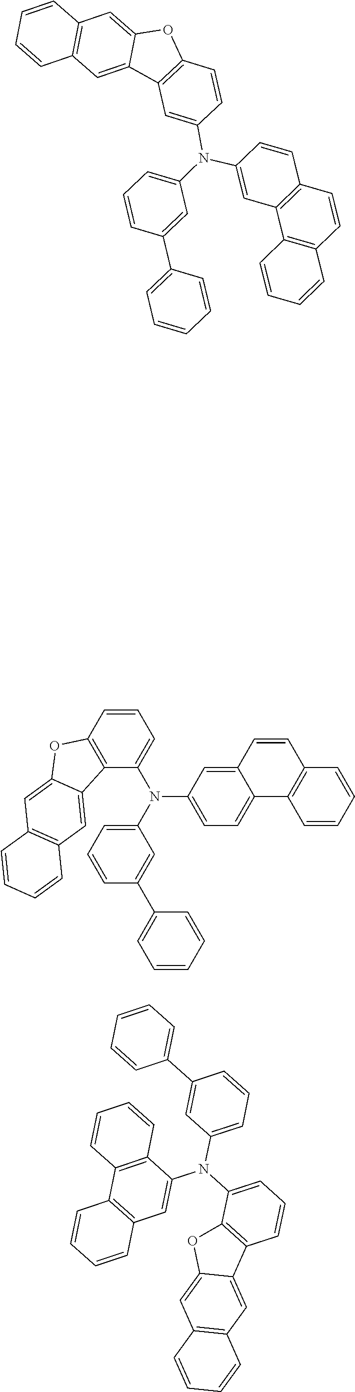 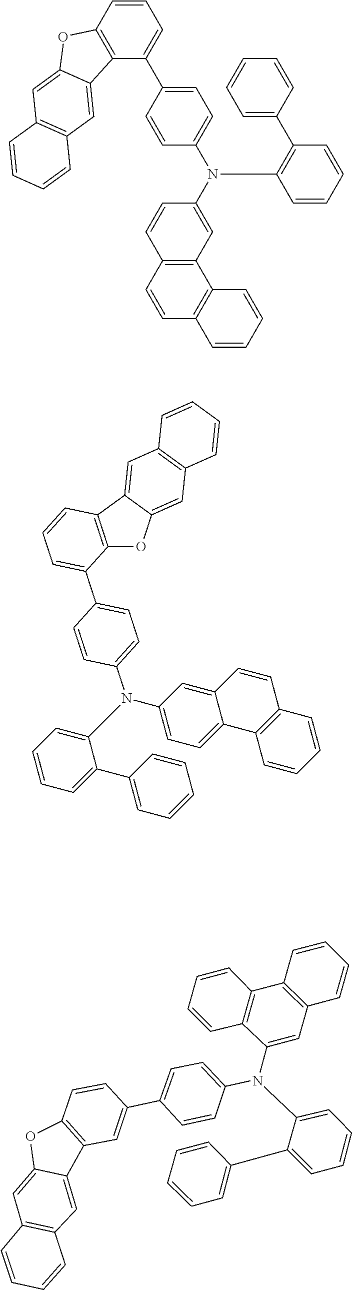 [SMILES: c1ccc(-c2ccccc2N(c2ccc(-c3cccc4oc5cc6ccccc6cc5c34)cc2)c2ccc3ccc4ccccc4c3c2)cc1 ; c1ccc(-c2ccccc2N(c2ccc(-c3cccc4c3oc3cc5ccccc5cc34)cc2)c2ccc3c(ccc4ccccc43)c2)cc1 ; c1ccc(-c2ccccc2N(c2ccc(-c3ccc4oc5cc6ccccc6cc5c4c3)cc2)c2cc3ccccc3c3ccccc23)cc1] 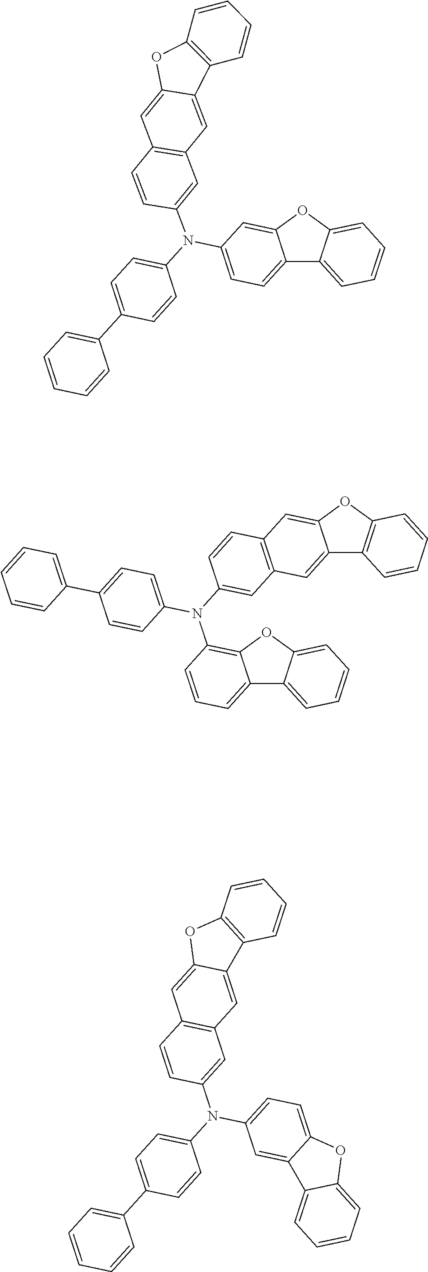 [SMILES: c1ccc(-c2ccc(N(c3ccc4cc5oc6ccccc6c5cc4c3)c3ccc4c(c3)oc3ccccc34)cc2)cc1 ; c1ccc(-c2ccc(N(c3ccc4cc5oc6ccccc6c5cc4c3)c3cccc4c3oc3ccccc34)cc2)cc1 ; c1ccc(-c2ccc(N(c3ccc4cc5oc6ccccc6c5cc4c3)c3ccc4oc5ccccc5c4c3)cc2)cc1] 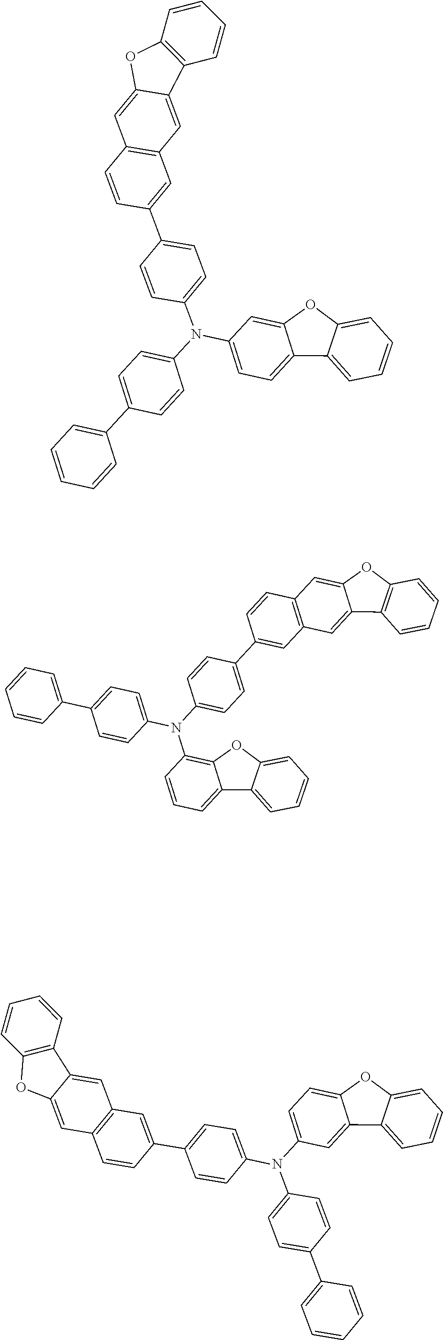 [SMILES: c1ccc(-c2ccc(N(c3ccc(-c4ccc5cc6oc7ccccc7c6cc5c4)cc3)c3ccc4c(c3)oc3ccccc34)cc2)cc1 ; c1ccc(-c2ccc(N(c3ccc(-c4ccc5cc6oc7ccccc7c6cc5c4)cc3)c3cccc4c3oc3ccccc34)cc2)cc1 ; c1ccc(-c2ccc(N(c3ccc(-c4ccc5cc6oc7ccccc7c6cc5c4)cc3)c3ccc4oc5ccccc5c4c3)cc2)cc1] 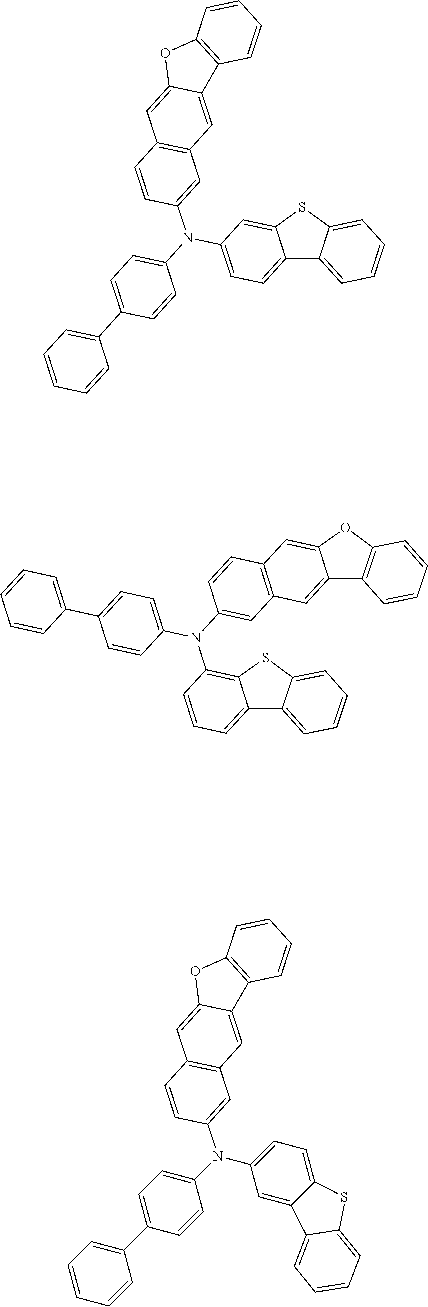 [SMILES: c1ccc(-c2ccc(N(c3ccc4cc5oc6ccccc6c5cc4c3)c3ccc4c(c3)sc3ccccc34)cc2)cc1 ; c1ccc(-c2ccc(N(c3ccc4cc5oc6ccccc6c5cc4c3)c3cccc4c3sc3ccccc34)cc2)cc1 ; c1ccc(-c2ccc(N(c3ccc4cc5oc6ccccc6c5cc4c3)c3ccc4sc5ccccc5c4c3)cc2)cc1] 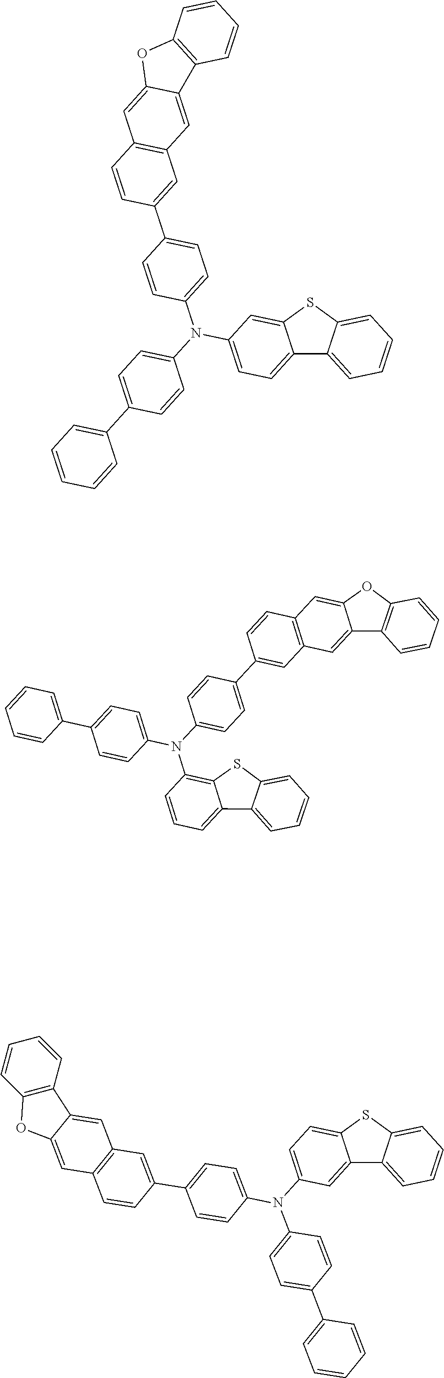 [SMILES: c1ccc(-c2ccc(N(c3ccc(-c4ccc5cc6oc7ccccc7c6cc5c4)cc3)c3ccc4c(c3)sc3ccccc34)cc2)cc1 ; c1ccc(-c2ccc(N(c3ccc(-c4ccc5cc6oc7ccccc7c6cc5c4)cc3)c3cccc4c3sc3ccccc34)cc2)cc1 ; c1ccc(-c2ccc(N(c3ccc(-c4ccc5cc6oc7ccccc7c6cc5c4)cc3)c3ccc4sc5ccccc5c4c3)cc2)cc1] 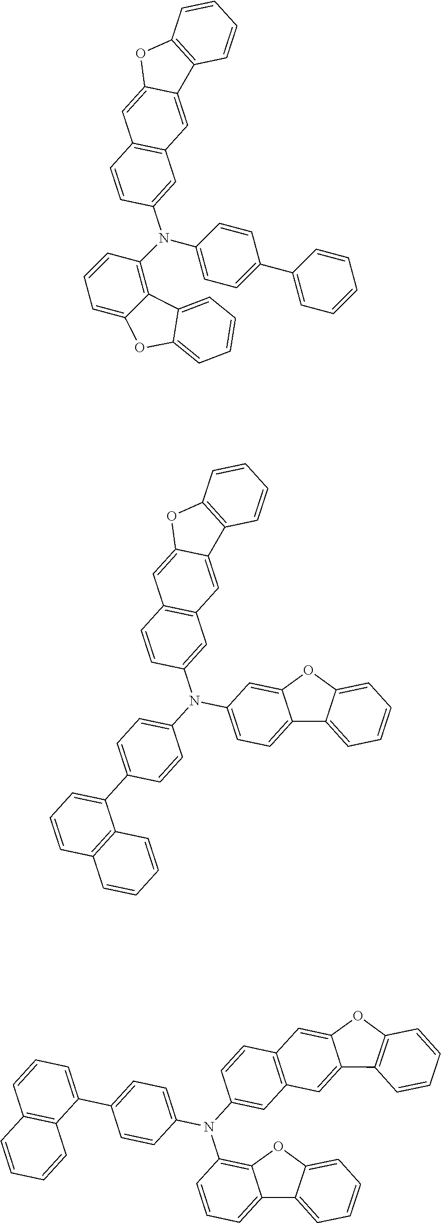 [SMILES: c1ccc(-c2ccc(N(c3ccc4cc5oc6ccccc6c5cc4c3)c3cccc4oc5ccccc5c34)cc2)cc1 ; c1ccc2c(-c3ccc(N(c4ccc5cc6oc7ccccc7c6cc5c4)c4ccc5c(c4)oc4ccccc45)cc3)cccc2c1 ; c1ccc2c(-c3ccc(N(c4ccc5cc6oc7ccccc7c6cc5c4)c4cccc5c4oc4ccccc45)cc3)cccc2c1] 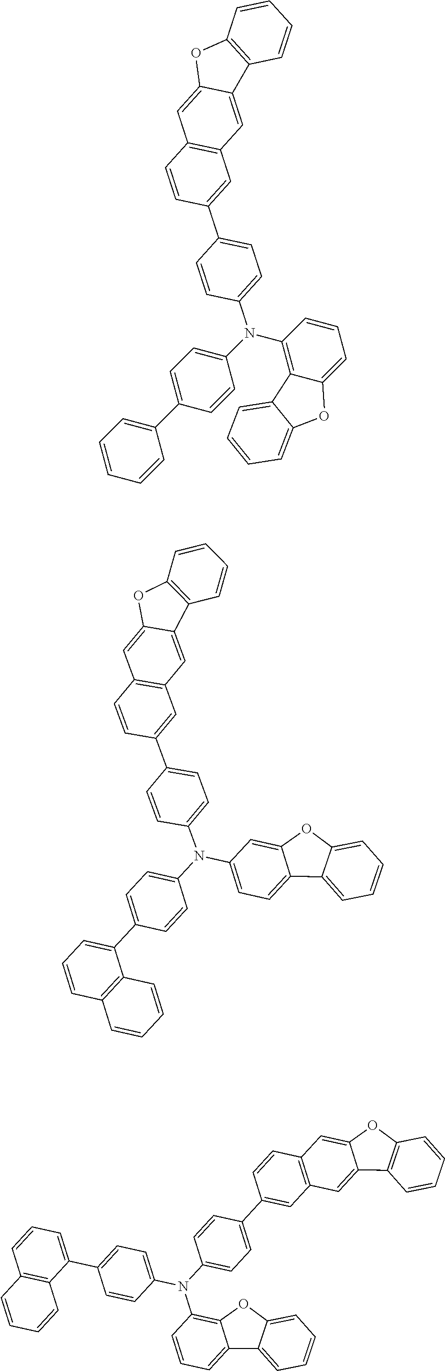 [SMILES: c1ccc(-c2ccc(N(c3ccc(-c4ccc5cc6oc7ccccc7c6cc5c4)cc3)c3cccc4oc5ccccc5c34)cc2)cc1 ; c1ccc2c(-c3ccc(N(c4ccc(-c5ccc6cc7oc8ccccc8c7cc6c5)cc4)c4ccc5c(c4)oc4ccccc45)cc3)cccc2c1 ; c1ccc2c(-c3ccc(N(c4ccc(-c5ccc6cc7oc8ccccc8c7cc6c5)cc4)c4cccc5c4oc4ccccc45)cc3)cccc2c1] 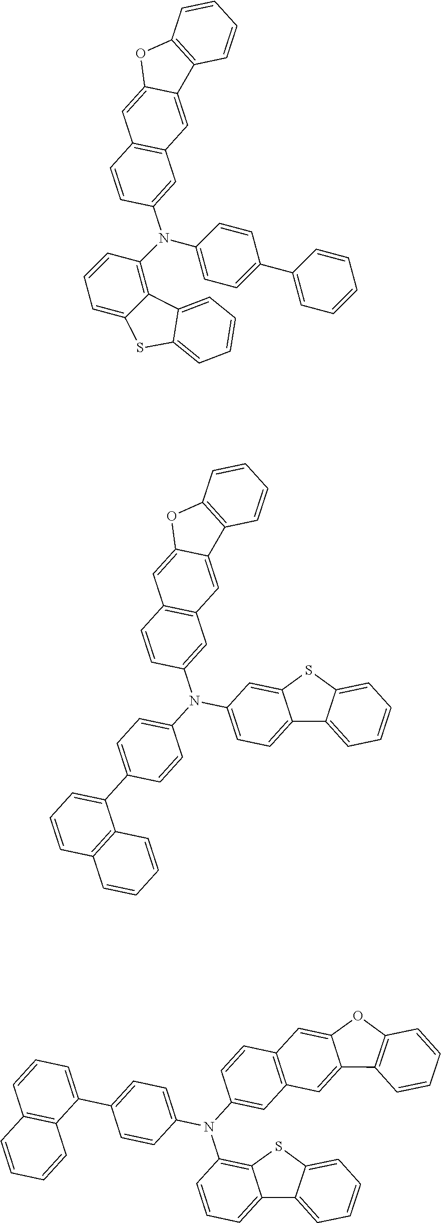 [SMILES: c1ccc(-c2ccc(N(c3ccc4cc5oc6ccccc6c5cc4c3)c3cccc4sc5ccccc5c34)cc2)cc1 ; c1ccc2c(-c3ccc(N(c4ccc5cc6oc7ccccc7c6cc5c4)c4ccc5c(c4)sc4ccccc45)cc3)cccc2c1 ; c1ccc2c(-c3ccc(N(c4ccc5cc6oc7ccccc7c6cc5c4)c4cccc5c4sc4ccccc45)cc3)cccc2c1] 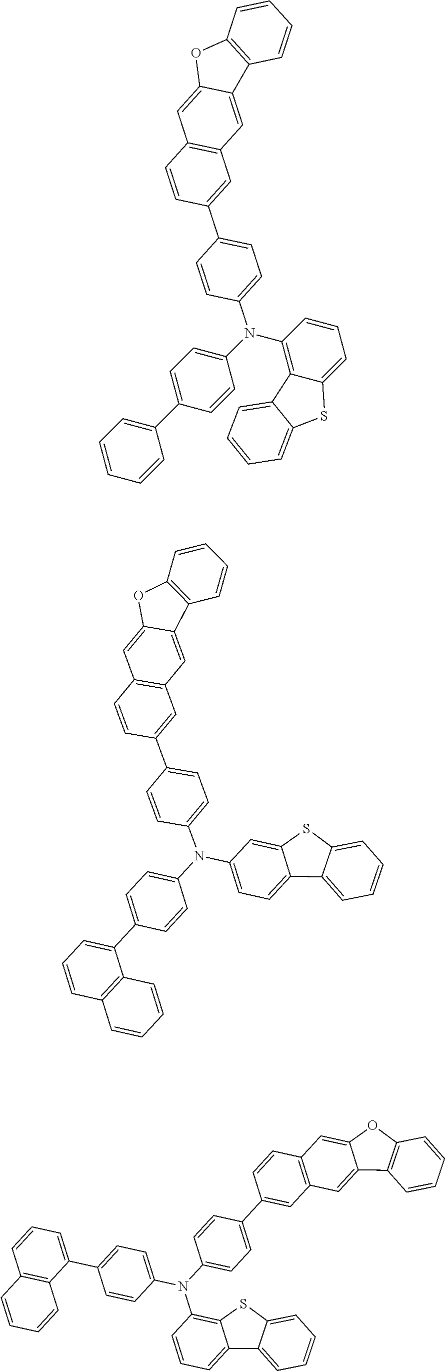 [SMILES: c1ccc(-c2ccc(N(c3ccc(-c4ccc5cc6oc7ccccc7c6cc5c4)cc3)c3cccc4sc5ccccc5c34)cc2)cc1 ; c1ccc2c(-c3ccc(N(c4ccc(-c5ccc6cc7oc8ccccc8c7cc6c5)cc4)c4ccc5c(c4)sc4ccccc45)cc3)cccc2c1 ; c1ccc2c(-c3ccc(N(c4ccc(-c5ccc6cc7oc8ccccc8c7cc6c5)cc4)c4cccc5c4sc4ccccc45)cc3)cccc2c1] 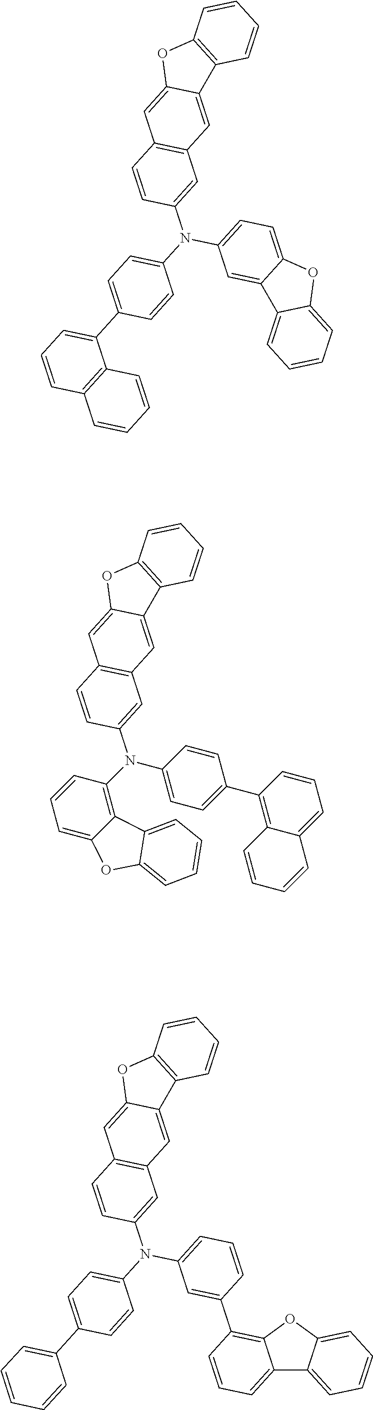 [SMILES: c1ccc2c(-c3ccc(N(c4ccc5cc6oc7ccccc7c6cc5c4)c4ccc5oc6ccccc6c5c4)cc3)cccc2c1 ; c1ccc2c(-c3ccc(N(c4ccc5cc6oc7ccccc7c6cc5c4)c4cccc5oc6ccccc6c45)cc3)cccc2c1 ; c1ccc(-c2ccc(N(c3cccc(-c4cccc5c4oc4ccccc45)c3)c3ccc4cc5oc6ccccc6c5cc4c3)cc2)cc1] 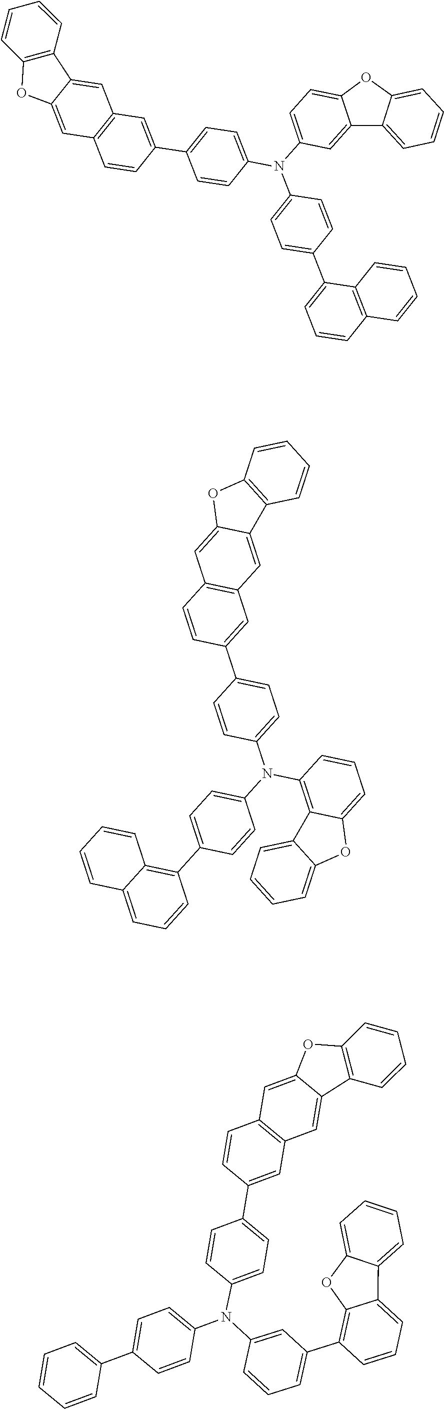 [SMILES: c1ccc2c(-c3ccc(N(c4ccc(-c5ccc6cc7oc8ccccc8c7cc6c5)cc4)c4ccc5oc6ccccc6c5c4)cc3)cccc2c1 ; c1ccc2c(-c3ccc(N(c4ccc(-c5ccc6cc7oc8ccccc8c7cc6c5)cc4)c4cccc5oc6ccccc6c45)cc3)cccc2c1 ; c1ccc(-c2ccc(N(c3ccc(-c4ccc5cc6oc7ccccc7c6cc5c4)cc3)c3cccc(-c4cccc5c4oc4ccccc45)c3)cc2)cc1] 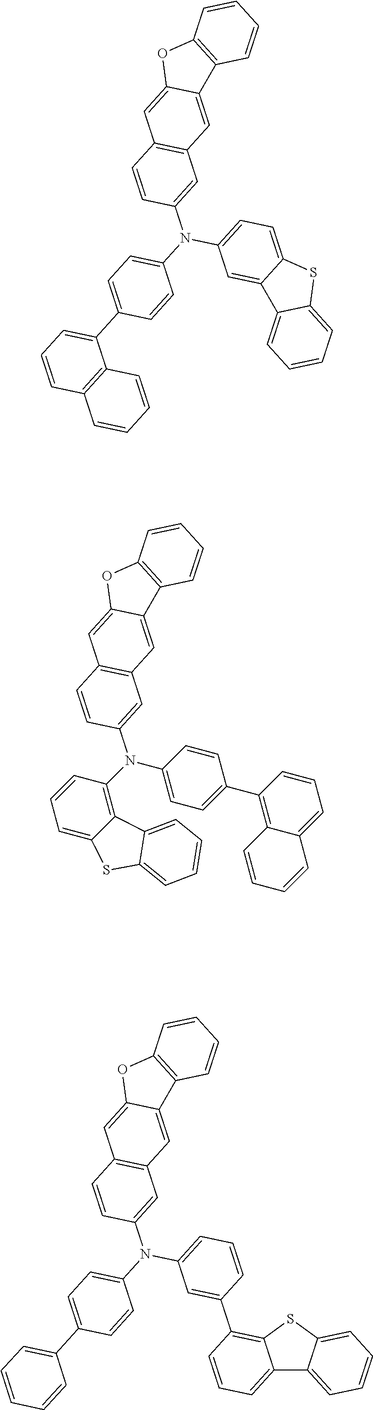 [SMILES: c1ccc2c(-c3ccc(N(c4ccc5cc6oc7ccccc7c6cc5c4)c4ccc5sc6ccccc6c5c4)cc3)cccc2c1 ; c1ccc2c(-c3ccc(N(c4ccc5cc6oc7ccccc7c6cc5c4)c4cccc5sc6ccccc6c45)cc3)cccc2c1 ; c1ccc(-c2ccc(N(c3cccc(-c4cccc5c4sc4ccccc45)c3)c3ccc4cc5oc6ccccc6c5cc4c3)cc2)cc1] 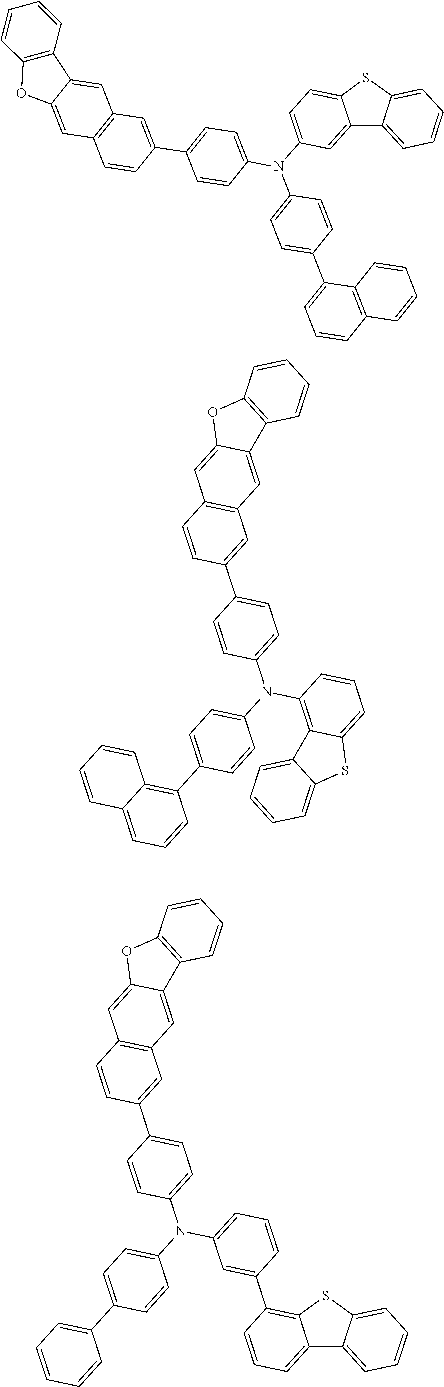 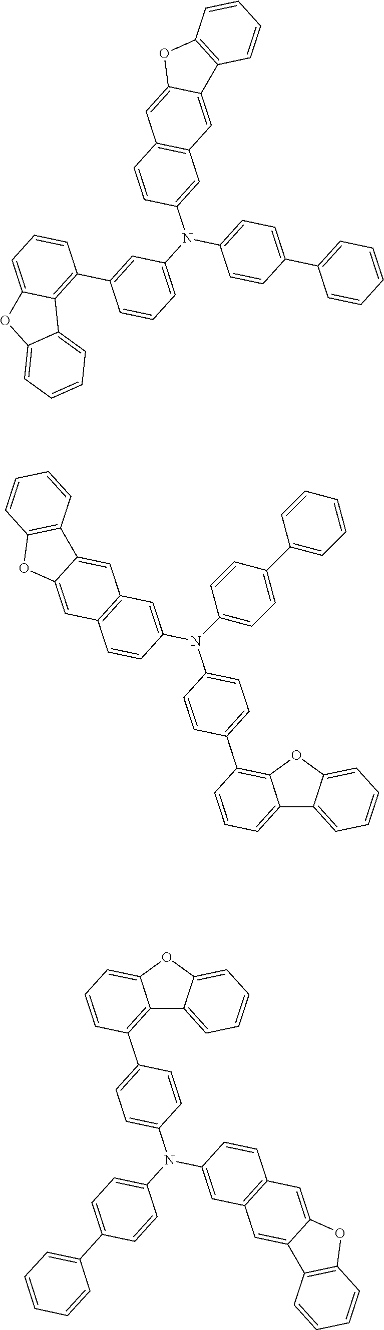 [SMILES: c1ccc(-c2ccc(N(c3cccc(-c4cccc5oc6ccccc6c45)c3)c3ccc4cc5oc6ccccc6c5cc4c3)cc2)cc1 ; c1ccc(-c2ccc(N(c3ccc(-c4cccc5c4oc4ccccc45)cc3)c3ccc4cc5oc6ccccc6c5cc4c3)cc2)cc1 ; c1ccc(-c2ccc(N(c3ccc(-c4cccc5oc6ccccc6c45)cc3)c3ccc4cc5oc6ccccc6c5cc4c3)cc2)cc1] 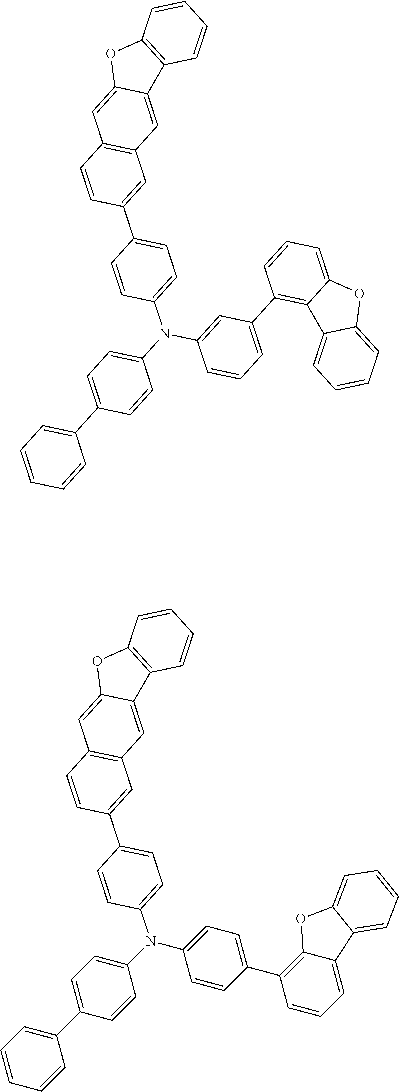 [SMILES: c1ccc(-c2ccc(N(c3ccc(-c4ccc5cc6oc7ccccc7c6cc5c4)cc3)c3cccc(-c4cccc5oc6ccccc6c45)c3)cc2)cc1 ; c1ccc(-c2ccc(N(c3ccc(-c4ccc5cc6oc7ccccc7c6cc5c4)cc3)c3ccc(-c4cccc5c4oc4ccccc45)cc3)cc2)cc1] 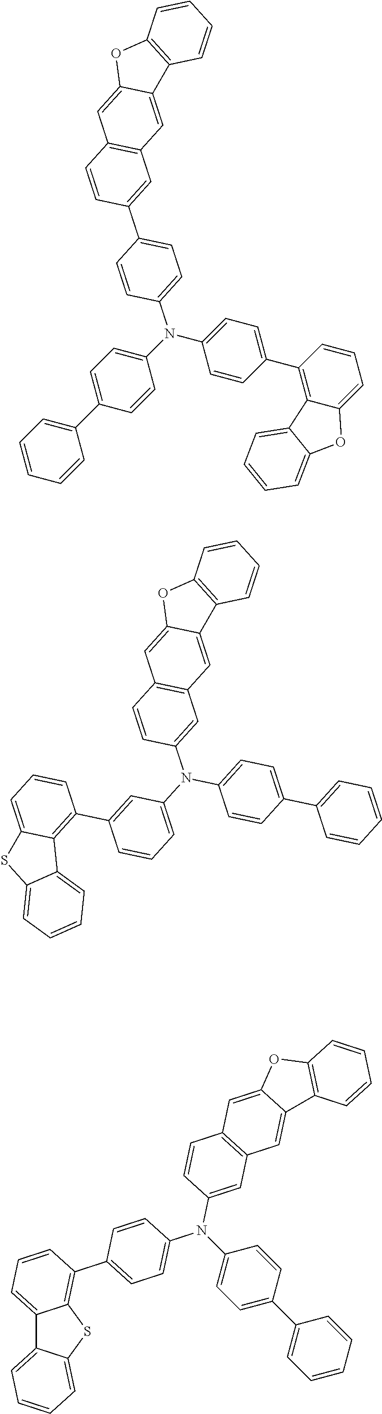 [SMILES: c1ccc(-c2ccc(N(c3ccc(-c4ccc5cc6oc7ccccc7c6cc5c4)cc3)c3ccc(-c4cccc5oc6ccccc6c45)cc3)cc2)cc1 ; c1ccc(-c2ccc(N(c3cccc(-c4cccc5sc6ccccc6c45)c3)c3ccc4cc5oc6ccccc6c5cc4c3)cc2)cc1 ; c1ccc(-c2ccc(N(c3ccc(-c4cccc5c4sc4ccccc45)cc3)c3ccc4cc5oc6ccccc6c5cc4c3)cc2)cc1] 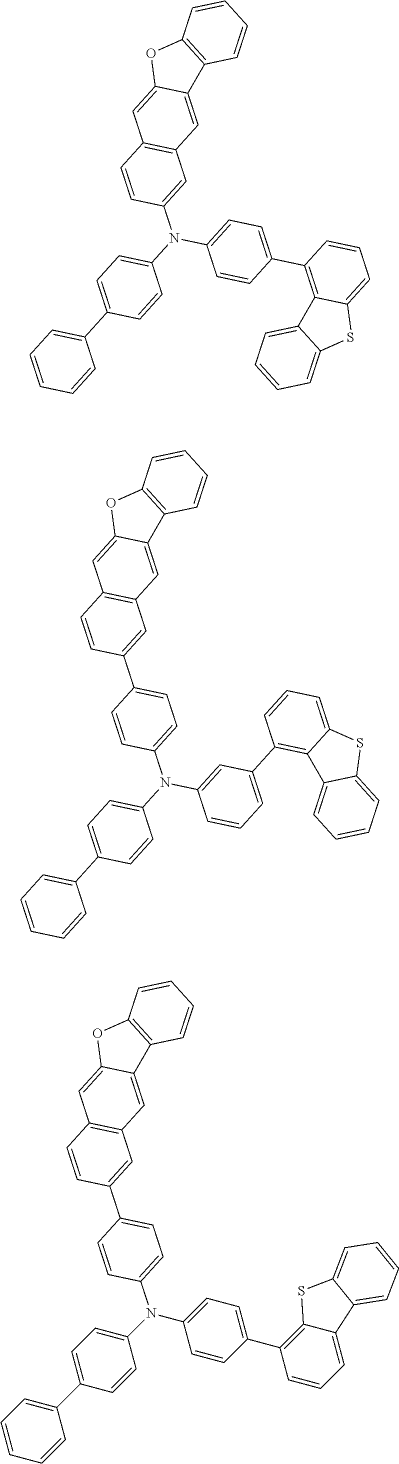 [SMILES: c1ccc(-c2ccc(N(c3ccc(-c4cccc5sc6ccccc6c45)cc3)c3ccc4cc5oc6ccccc6c5cc4c3)cc2)cc1 ; c1ccc(-c2ccc(N(c3ccc(-c4ccc5cc6oc7ccccc7c6cc5c4)cc3)c3cccc(-c4cccc5sc6ccccc6c45)c3)cc2)cc1 ; c1ccc(-c2ccc(N(c3ccc(-c4ccc5cc6oc7ccccc7c6cc5c4)cc3)c3ccc(-c4cccc5c4sc4ccccc45)cc3)cc2)cc1] 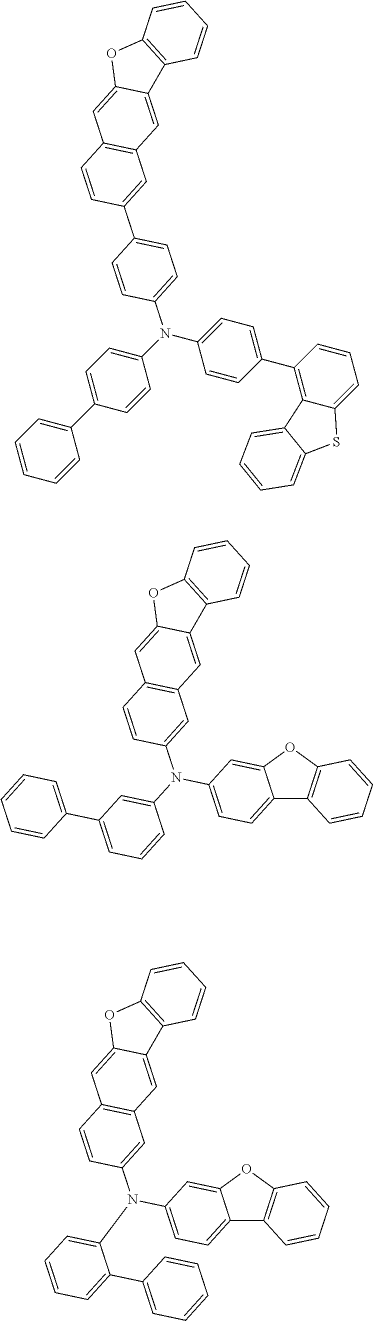 [SMILES: c1ccc(-c2ccc(N(c3ccc(-c4ccc5cc6oc7ccccc7c6cc5c4)cc3)c3ccc(-c4cccc5sc6ccccc6c45)cc3)cc2)cc1 ; c1ccc(-c2cccc(N(c3ccc4cc5oc6ccccc6c5cc4c3)c3ccc4c(c3)oc3ccccc34)c2)cc1 ; c1ccc(-c2ccccc2N(c2ccc3cc4oc5ccccc5c4cc3c2)c2ccc3c(c2)oc2ccccc23)cc1] 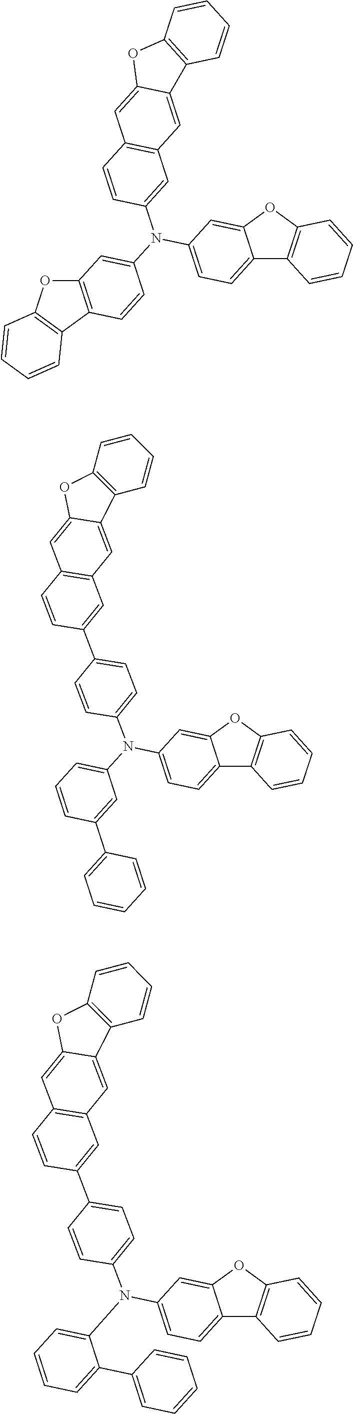 [SMILES: c1ccc2c(c1)oc1cc(N(c3ccc4cc5oc6ccccc6c5cc4c3)c3ccc4c(c3)oc3ccccc34)ccc12 ; c1ccc(-c2cccc(N(c3ccc(-c4ccc5cc6oc7ccccc7c6cc5c4)cc3)c3ccc4c(c3)oc3ccccc34)c2)cc1 ; c1ccc(-c2ccccc2N(c2ccc(-c3ccc4cc5oc6ccccc6c5cc4c3)cc2)c2ccc3c(c2)oc2ccccc23)cc1] 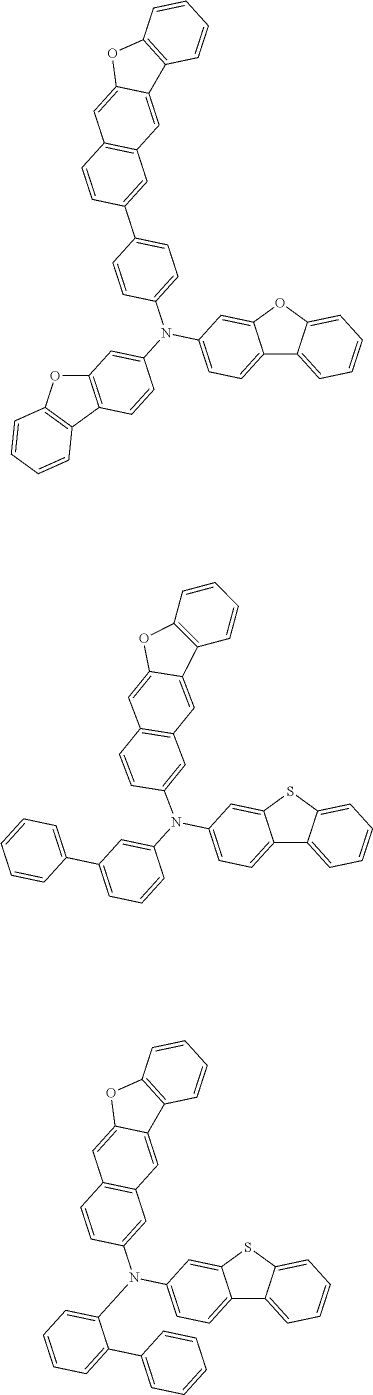 [SMILES: c1ccc2c(c1)oc1cc(N(c3ccc(-c4ccc5cc6oc7ccccc7c6cc5c4)cc3)c3ccc4c(c3)oc3ccccc34)ccc12 ; c1ccc(-c2cccc(N(c3ccc4cc5oc6ccccc6c5cc4c3)c3ccc4c(c3)sc3ccccc34)c2)cc1 ; c1ccc(-c2ccccc2N(c2ccc3cc4oc5ccccc5c4cc3c2)c2ccc3c(c2)sc2ccccc23)cc1] 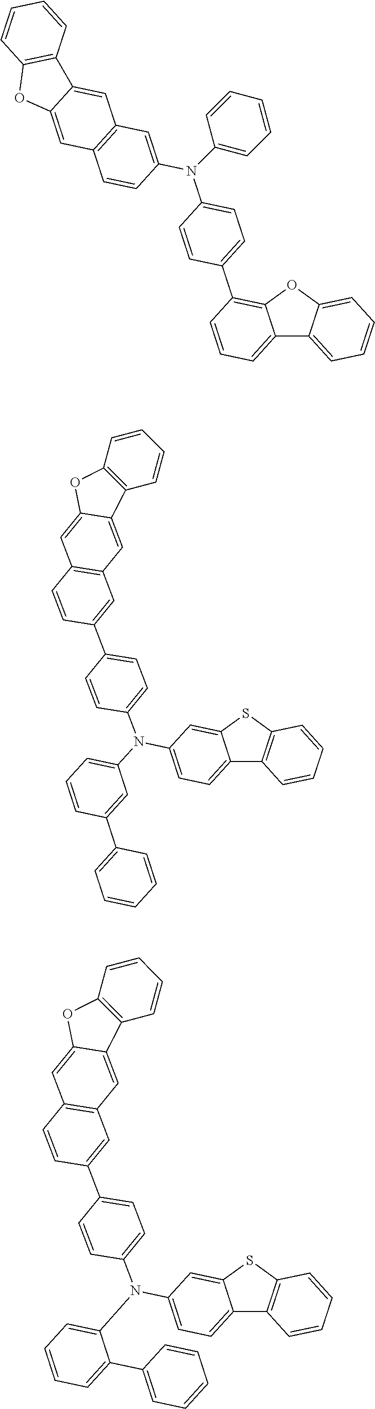 [SMILES: c1ccc(N(c2ccc(-c3cccc4c3oc3ccccc34)cc2)c2ccc3cc4oc5ccccc5c4cc3c2)cc1 ; c1ccc(-c2cccc(N(c3ccc(-c4ccc5cc6oc7ccccc7c6cc5c4)cc3)c3ccc4c(c3)sc3ccccc34)c2)cc1 ; c1ccc(-c2ccccc2N(c2ccc(-c3ccc4cc5oc6ccccc6c5cc4c3)cc2)c2ccc3c(c2)sc2ccccc23)cc1] 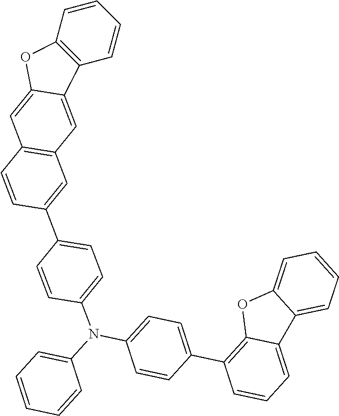 [SMILES: c1ccc(N(c2ccc(-c3ccc4cc5oc6ccccc6c5cc4c3)cc2)c2ccc(-c3cccc4c3oc3ccccc34)cc2)cc1] 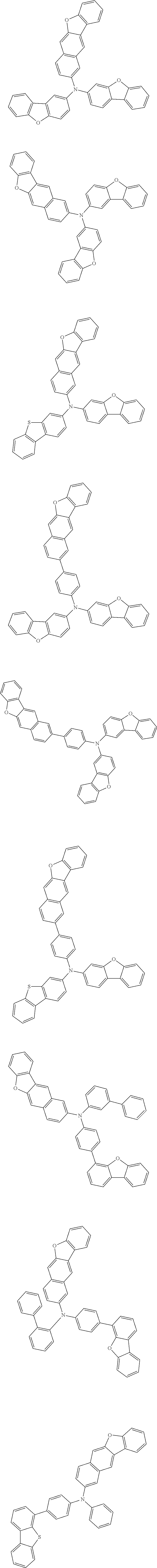 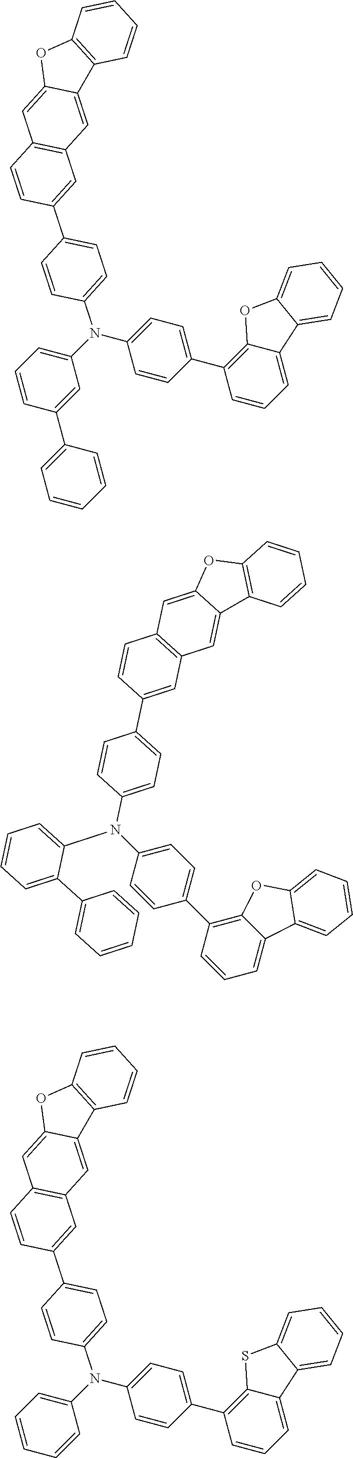 [SMILES: c1ccc(-c2cccc(N(c3ccc(-c4ccc5cc6oc7ccccc7c6cc5c4)cc3)c3ccc(-c4cccc5c4oc4ccccc45)cc3)c2)cc1 ; c1ccc(-c2ccccc2N(c2ccc(-c3ccc4cc5oc6ccccc6c5cc4c3)cc2)c2ccc(-c3cccc4c3oc3ccccc34)cc2)cc1 ; c1ccc(N(c2ccc(-c3ccc4cc5oc6ccccc6c5cc4c3)cc2)c2ccc(-c3cccc4c3sc3ccccc34)cc2)cc1] 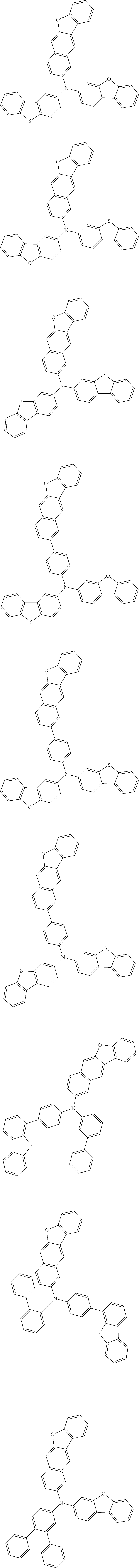 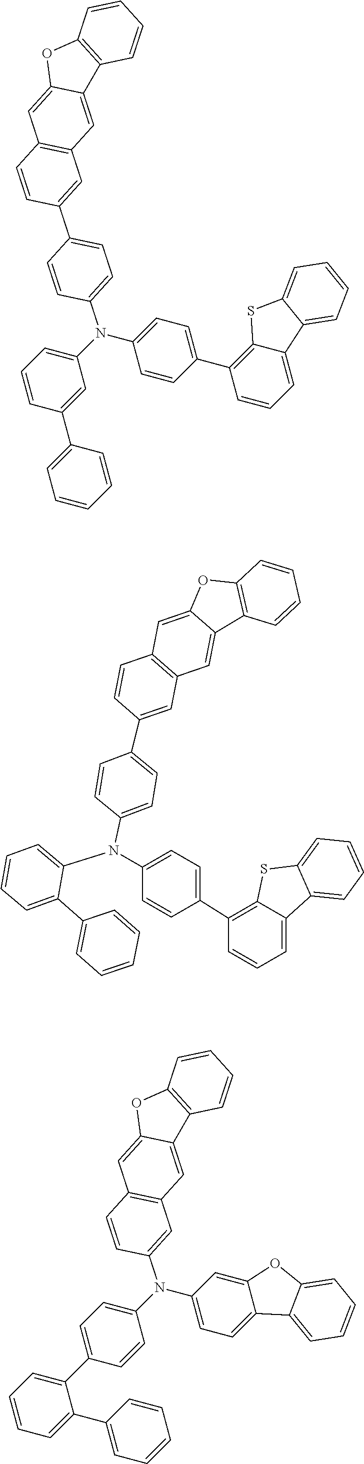 [SMILES: c1ccc(-c2cccc(N(c3ccc(-c4ccc5cc6oc7ccccc7c6cc5c4)cc3)c3ccc(-c4cccc5c4sc4ccccc45)cc3)c2)cc1 ; c1ccc(-c2ccccc2N(c2ccc(-c3ccc4cc5oc6ccccc6c5cc4c3)cc2)c2ccc(-c3cccc4c3sc3ccccc34)cc2)cc1 ; c1ccc(-c2ccccc2-c2ccc(N(c3ccc4cc5oc6ccccc6c5cc4c3)c3ccc4c(c3)oc3ccccc34)cc2)cc1] 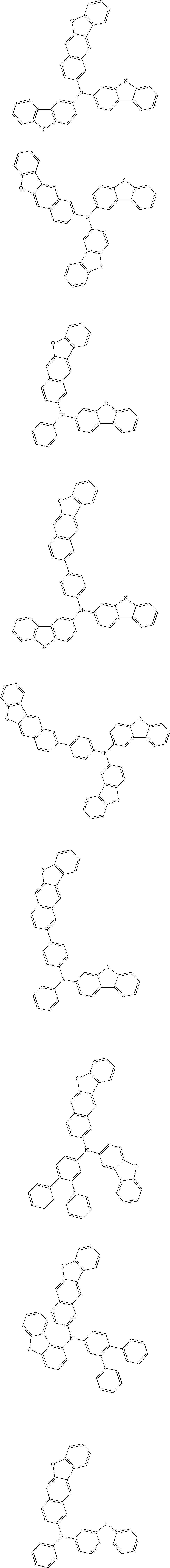 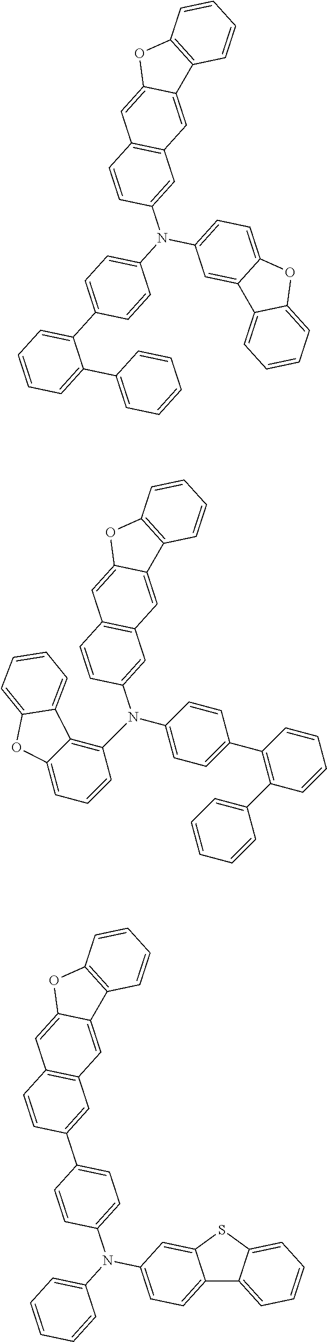 [SMILES: c1ccc(-c2ccccc2-c2ccc(N(c3ccc4cc5oc6ccccc6c5cc4c3)c3ccc4oc5ccccc5c4c3)cc2)cc1 ; c1ccc(-c2ccccc2-c2ccc(N(c3ccc4cc5oc6ccccc6c5cc4c3)c3cccc4oc5ccccc5c34)cc2)cc1 ; c1ccc(N(c2ccc(-c3ccc4cc5oc6ccccc6c5cc4c3)cc2)c2ccc3c(c2)sc2ccccc23)cc1] 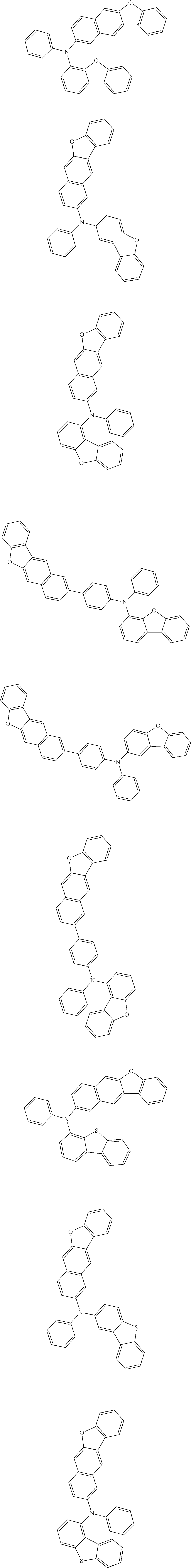 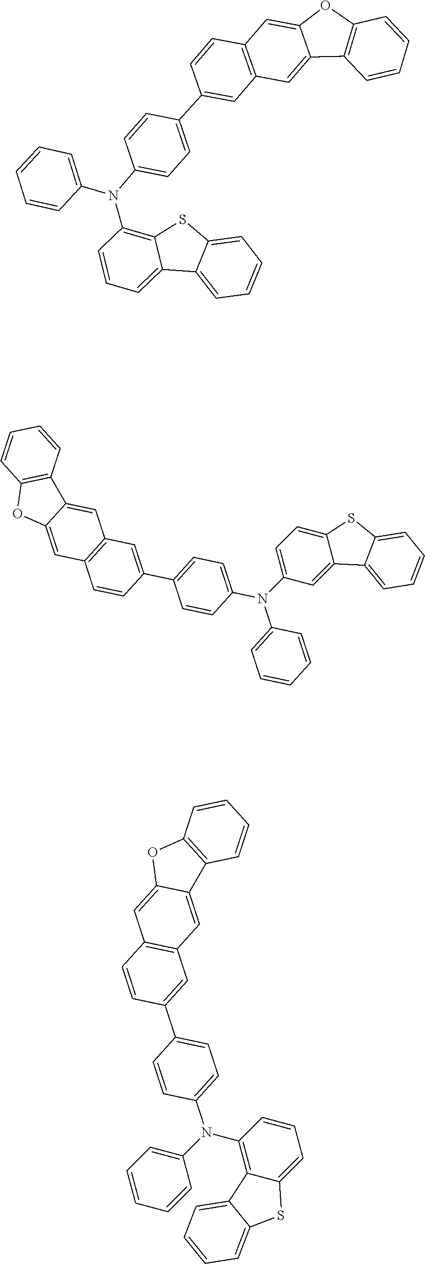 [SMILES: c1ccc(N(c2ccc(-c3ccc4cc5oc6ccccc6c5cc4c3)cc2)c2cccc3c2sc2ccccc23)cc1 ; c1ccc(N(c2ccc(-c3ccc4cc5oc6ccccc6c5cc4c3)cc2)c2ccc3sc4ccccc4c3c2)cc1 ; c1ccc(N(c2ccc(-c3ccc4cc5oc6ccccc6c5cc4c3)cc2)c2cccc3sc4ccccc4c23)cc1] 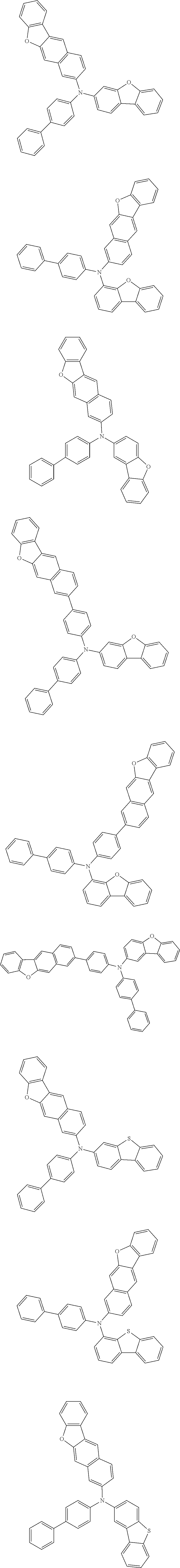 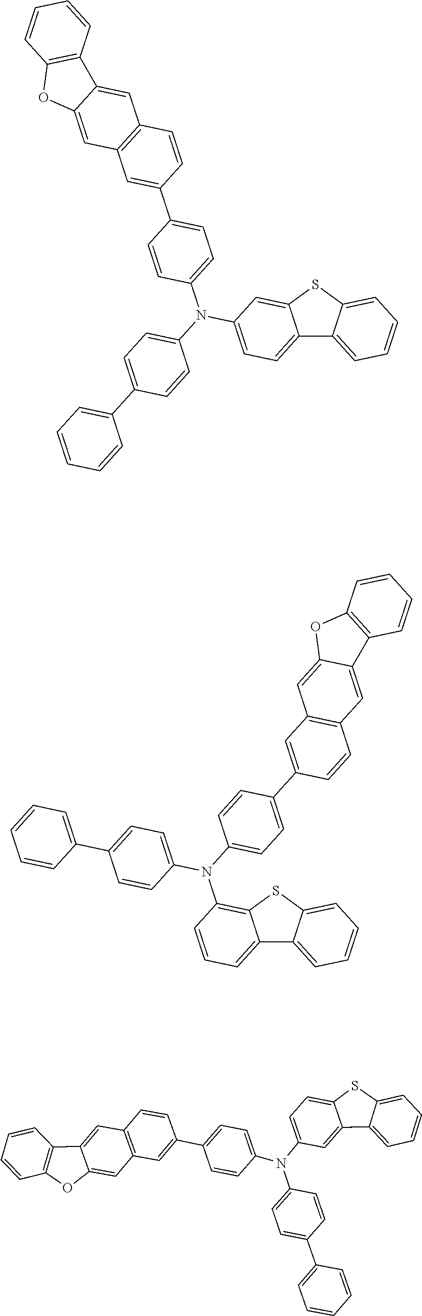 [SMILES: c1ccc(-c2ccc(N(c3ccc(-c4ccc5cc6c(cc5c4)oc4ccccc46)cc3)c3ccc4c(c3)sc3ccccc34)cc2)cc1 ; c1ccc(-c2ccc(N(c3ccc(-c4ccc5cc6c(cc5c4)oc4ccccc46)cc3)c3cccc4c3sc3ccccc34)cc2)cc1 ; c1ccc(-c2ccc(N(c3ccc(-c4ccc5cc6c(cc5c4)oc4ccccc46)cc3)c3ccc4sc5ccccc5c4c3)cc2)cc1] 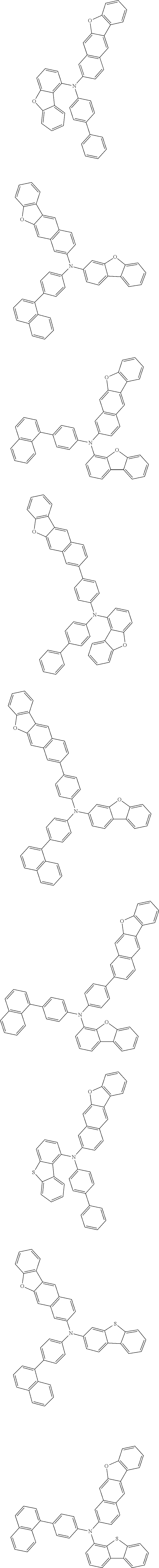 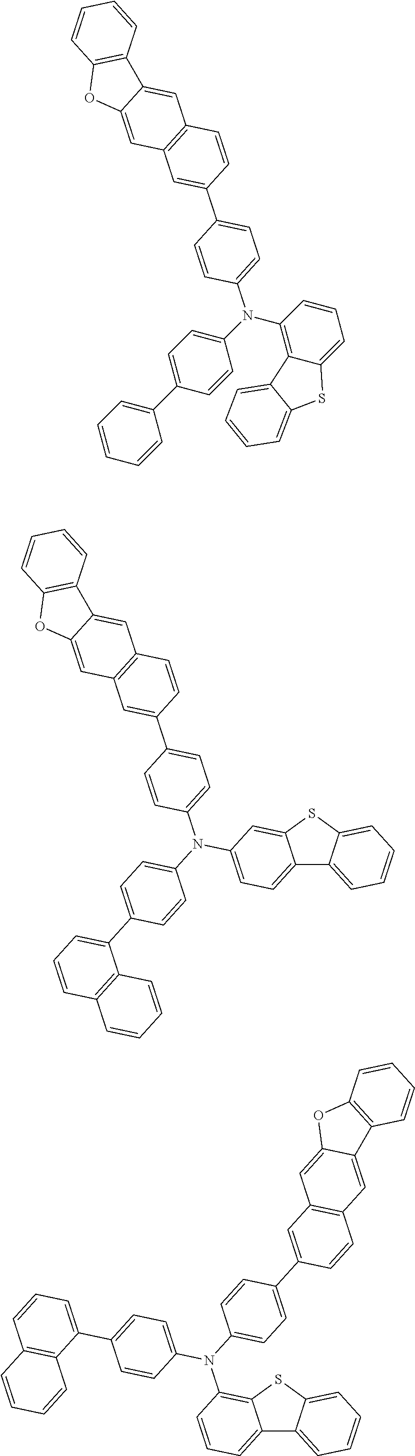 [SMILES: c1ccc(-c2ccc(N(c3ccc(-c4ccc5cc6c(cc5c4)oc4ccccc46)cc3)c3cccc4sc5ccccc5c34)cc2)cc1 ; c1ccc2c(-c3ccc(N(c4ccc(-c5ccc6cc7c(cc6c5)oc5ccccc57)cc4)c4ccc5c(c4)sc4ccccc45)cc3)cccc2c1 ; c1ccc2c(-c3ccc(N(c4ccc(-c5ccc6cc7c(cc6c5)oc5ccccc57)cc4)c4cccc5c4sc4ccccc45)cc3)cccc2c1] 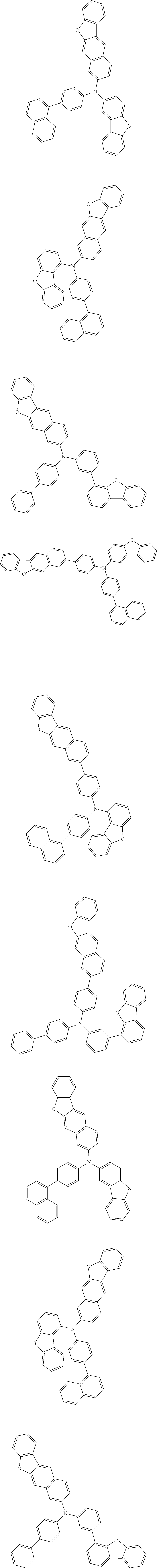 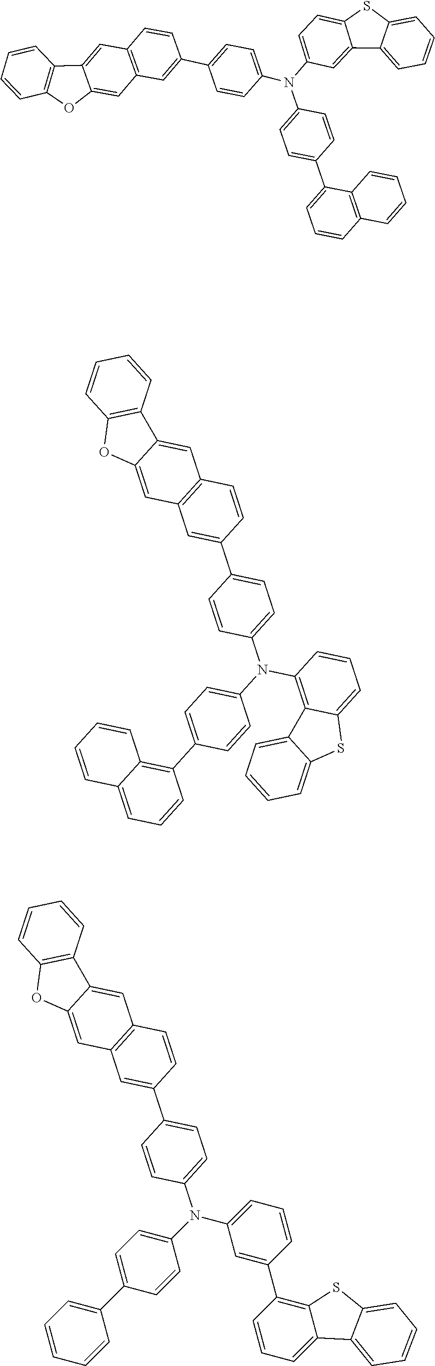 [SMILES: c1ccc2c(-c3ccc(N(c4ccc(-c5ccc6cc7c(cc6c5)oc5ccccc57)cc4)c4ccc5sc6ccccc6c5c4)cc3)cccc2c1 ; c1ccc2c(-c3ccc(N(c4ccc(-c5ccc6cc7c(cc6c5)oc5ccccc57)cc4)c4cccc5sc6ccccc6c45)cc3)cccc2c1 ; c1ccc(-c2ccc(N(c3ccc(-c4ccc5cc6c(cc5c4)oc4ccccc46)cc3)c3cccc(-c4cccc5c4sc4ccccc45)c3)cc2)cc1] 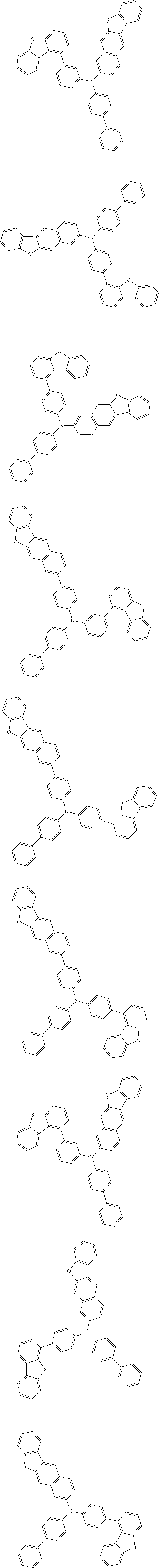 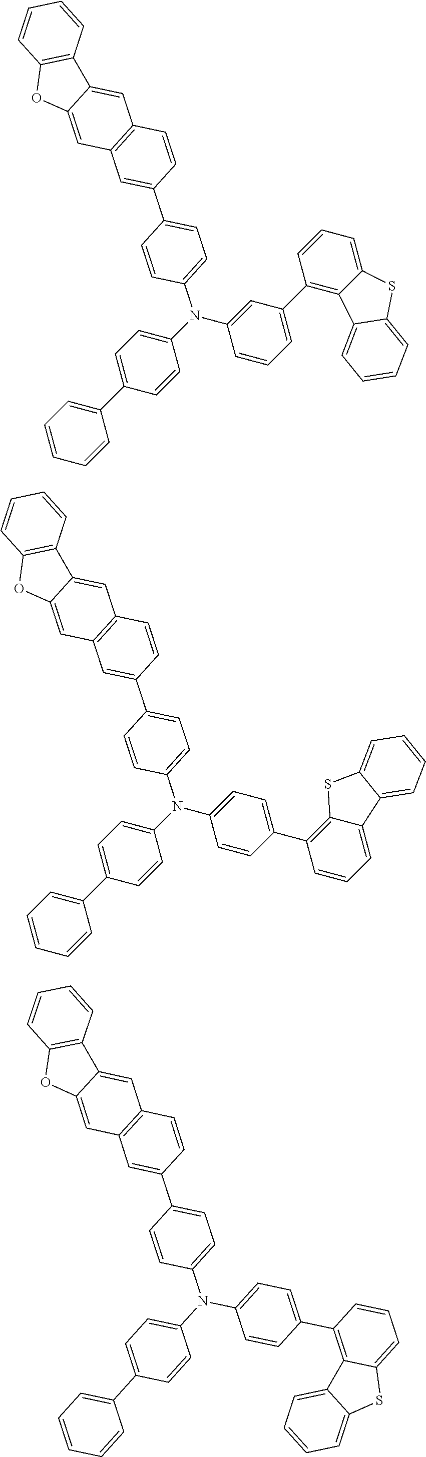 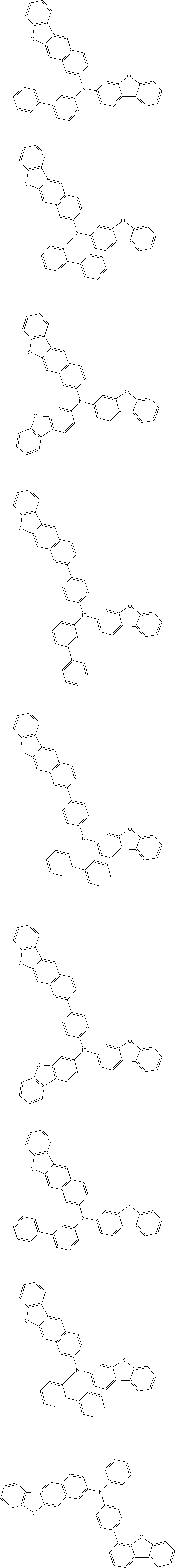 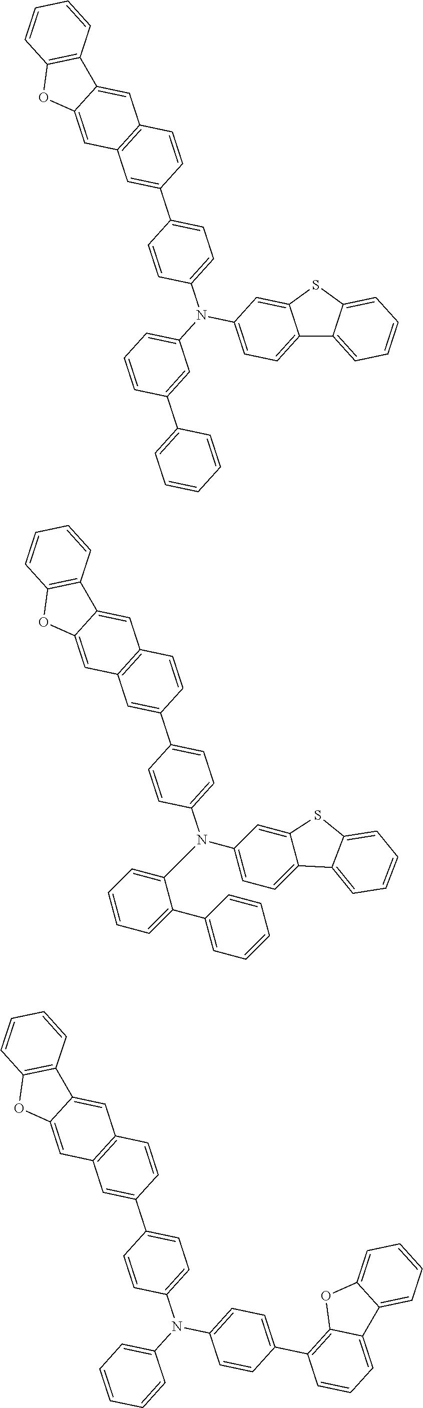 [SMILES: c1ccc(-c2cccc(N(c3ccc(-c4ccc5cc6c(cc5c4)oc4ccccc46)cc3)c3ccc4c(c3)sc3ccccc34)c2)cc1 ; c1ccc(-c2ccccc2N(c2ccc(-c3ccc4cc5c(cc4c3)oc3ccccc35)cc2)c2ccc3c(c2)sc2ccccc23)cc1 ; c1ccc(N(c2ccc(-c3ccc4cc5c(cc4c3)oc3ccccc35)cc2)c2ccc(-c3cccc4c3oc3ccccc34)cc2)cc1] 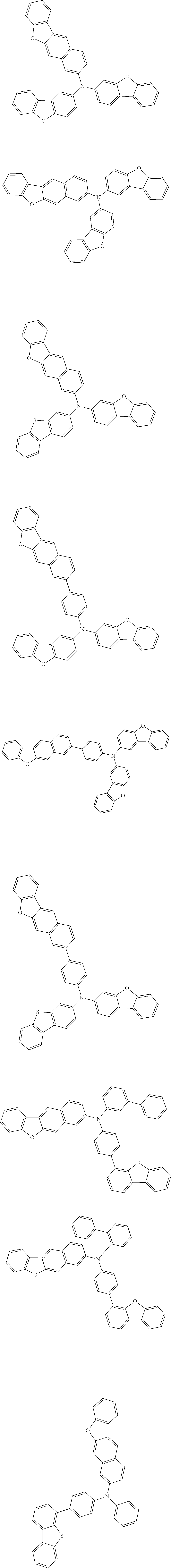 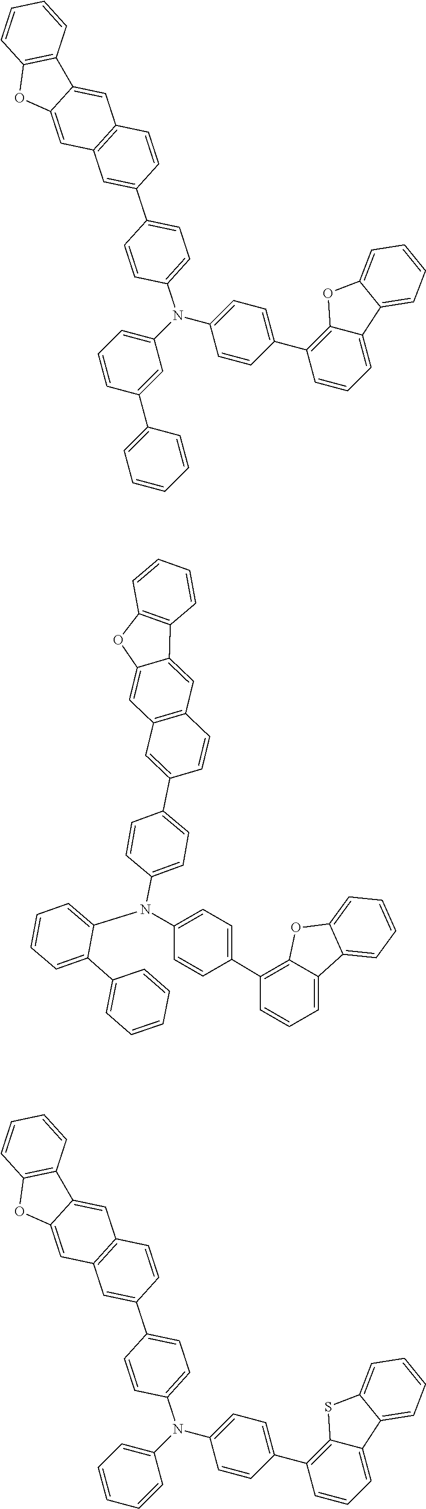 [SMILES: c1ccc(-c2cccc(N(c3ccc(-c4ccc5cc6c(cc5c4)oc4ccccc46)cc3)c3ccc(-c4cccc5c4oc4ccccc45)cc3)c2)cc1 ; c1ccc(-c2ccccc2N(c2ccc(-c3ccc4cc5c(cc4c3)oc3ccccc35)cc2)c2ccc(-c3cccc4c3oc3ccccc34)cc2)cc1 ; c1ccc(N(c2ccc(-c3ccc4cc5c(cc4c3)oc3ccccc35)cc2)c2ccc(-c3cccc4c3sc3ccccc34)cc2)cc1] 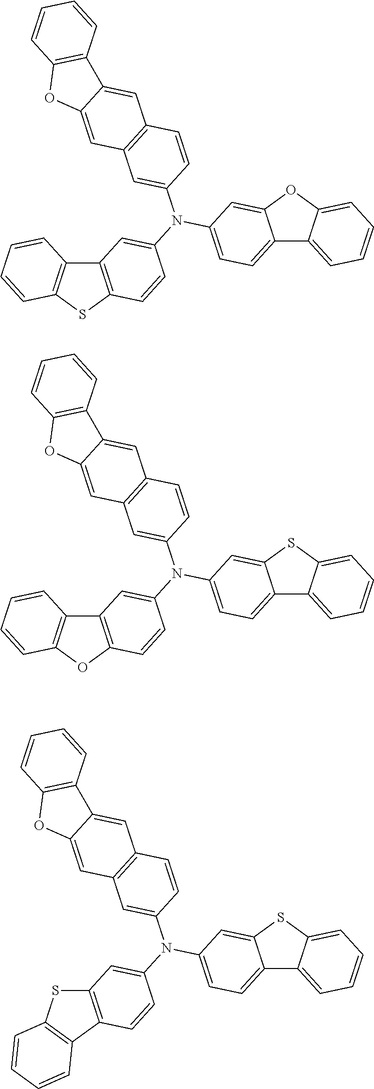 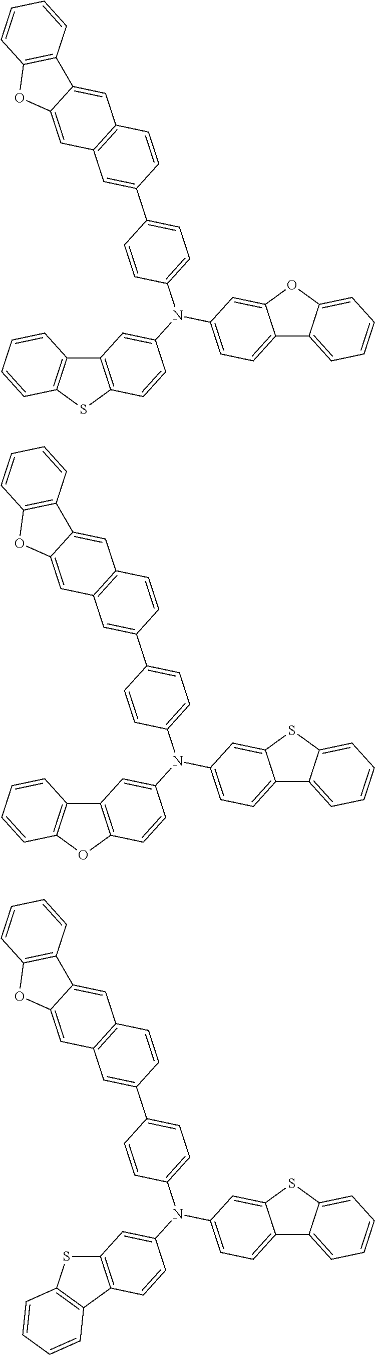 [SMILES: c1ccc2c(c1)oc1cc(N(c3ccc(-c4ccc5cc6c(cc5c4)oc4ccccc46)cc3)c3ccc4sc5ccccc5c4c3)ccc12 ; c1ccc2c(c1)oc1ccc(N(c3ccc(-c4ccc5cc6c(cc5c4)oc4ccccc46)cc3)c3ccc4c(c3)sc3ccccc34)cc12 ; c1ccc2c(c1)oc1cc3cc(-c4ccc(N(c5ccc6c(c5)sc5ccccc56)c5ccc6c(c5)sc5ccccc56)cc4)ccc3cc12] 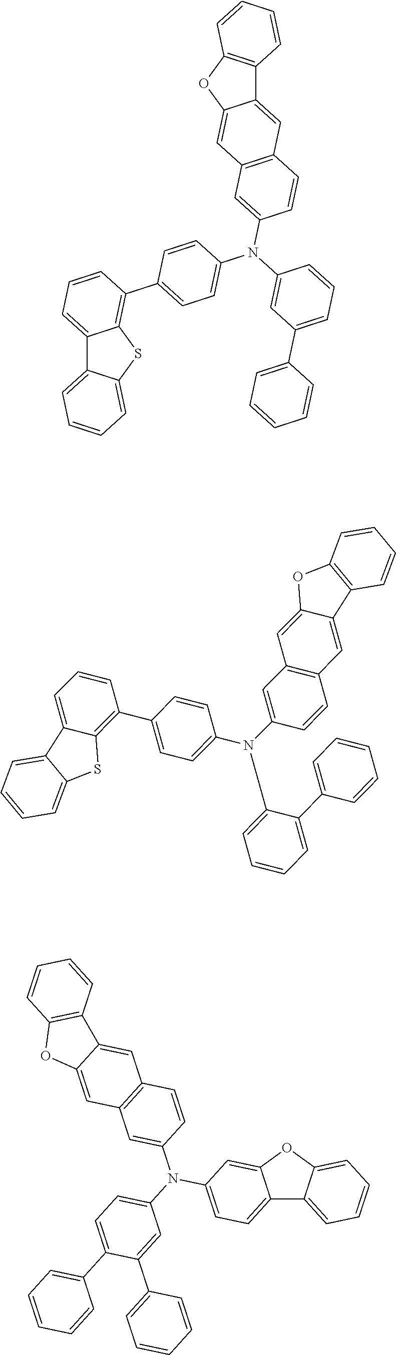 [SMILES: c1ccc(-c2cccc(N(c3ccc(-c4cccc5c4sc4ccccc45)cc3)c3ccc4cc5c(cc4c3)oc3ccccc35)c2)cc1 ; c1ccc(-c2ccccc2CN(c2ccc(-c3cccc4c3sc3ccccc34)cc2)c2ccc3cc4c(cc3c2)oc2ccccc24)cc1 ; c1ccc(-c2ccc(N(c3ccc4cc5c(cc4c3)oc3ccccc35)c3ccc4c(c3)oc3ccccc34)cc2-c2ccccc2)cc1] 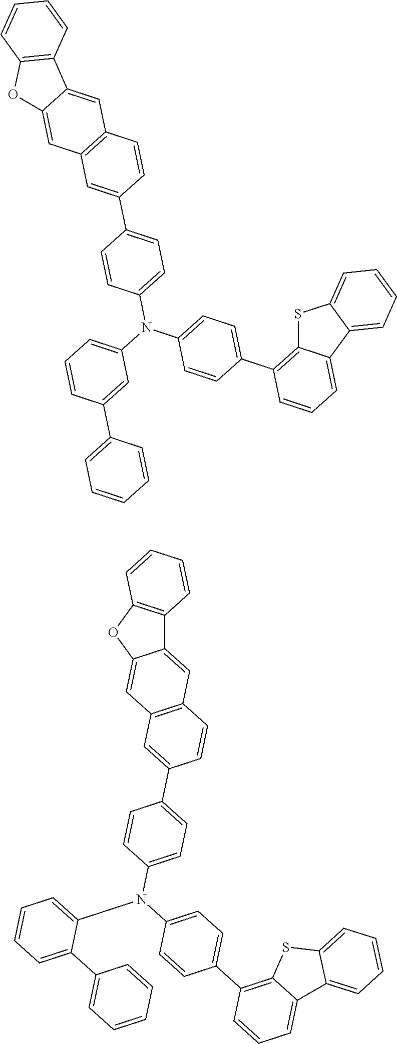 [SMILES: c1ccc(-c2cccc(N(c3ccc(-c4ccc5cc6c(cc5c4)oc4ccccc46)cc3)c3ccc(-c4cccc5c4sc4ccccc45)cc3)c2)cc1 ; c1ccc(-c2ccccc2CN(c2ccc(-c3ccc4cc5c(cc4c3)oc3ccccc35)cc2)c2ccc(-c3cccc4c3sc3ccccc34)cc2)cc1] 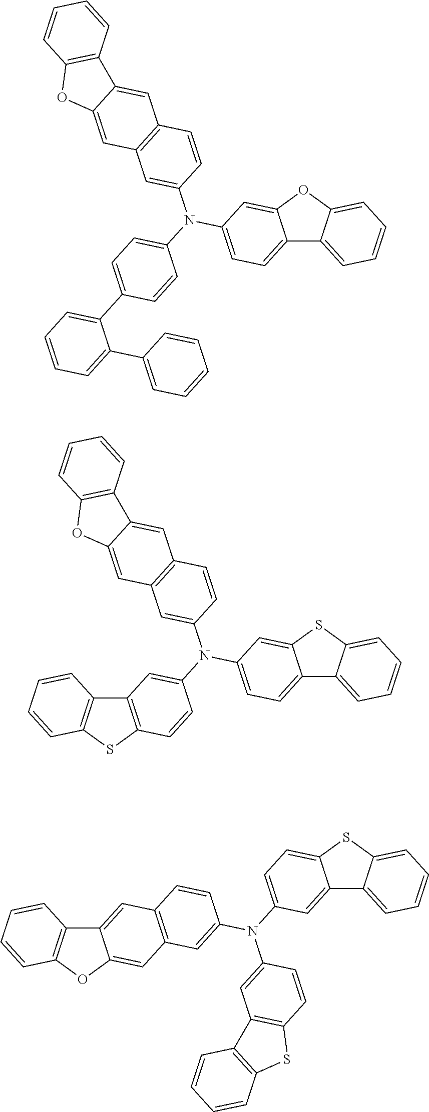 [SMILES: c1ccc(-c2ccccc2-c2ccc(N(c3ccc4cc5c(cc4c3)oc3ccccc35)c3ccc4c(c3)oc3ccccc34)cc2)cc1 ; c1ccc2c(c1)oc1cc3cc(N(c4ccc5c(c4)sc4ccccc45)c4ccc5sc6ccccc6c5c4)ccc3cc12 ; c1ccc2c(c1)oc1cc3cc(N(c4ccc5sc6ccccc6c5c4)c4ccc5sc6ccccc6c5c4)ccc3cc12] 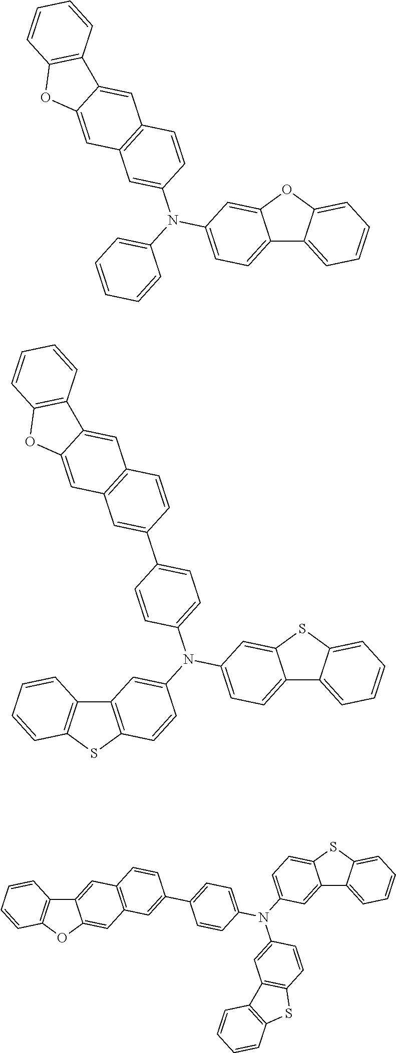 [SMILES: c1ccc(N(c2ccc3cc4c(cc3c2)oc2ccccc24)c2ccc3c(c2)oc2ccccc23)cc1 ; c1ccc2c(c1)oc1cc3cc(-c4ccc(N(c5ccc6c(c5)sc5ccccc56)c5ccc6sc7ccccc7c6c5)cc4)ccc3cc12 ; c1ccc2c(c1)oc1cc3cc(-c4ccc(N(c5ccc6sc7ccccc7c6c5)c5ccc6sc7ccccc7c6c5)cc4)ccc3cc12] 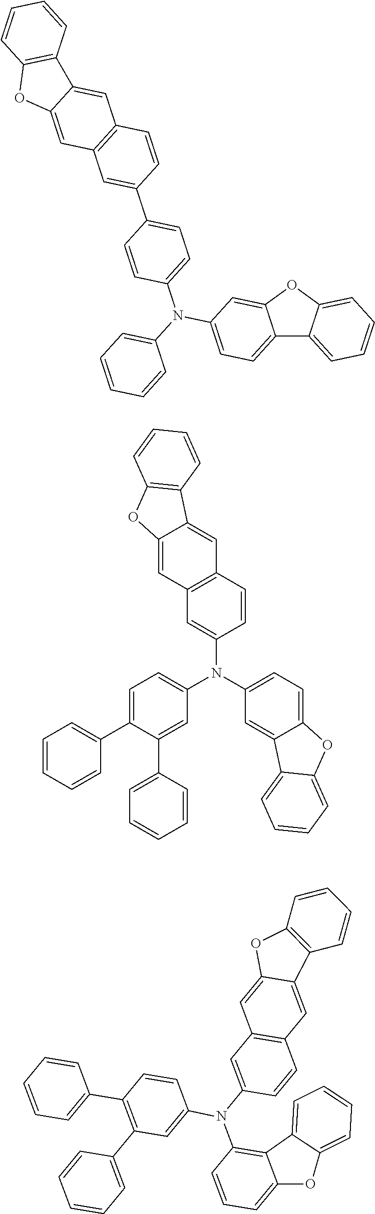 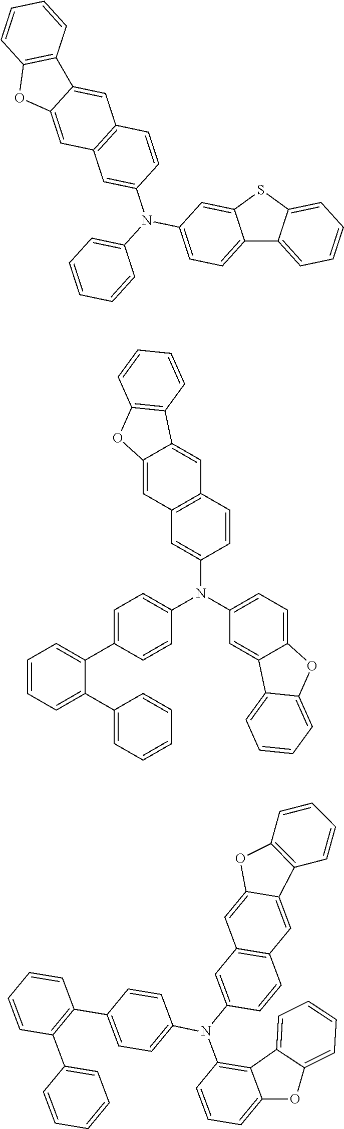 [SMILES: c1ccc(N(c2ccc3cc4c(cc3c2)oc2ccccc24)c2ccc3c(c2)sc2ccccc23)cc1 ; c1ccc(-c2ccccc2-c2ccc(N(c3ccc4cc5c(cc4c3)oc3ccccc35)c3ccc4oc5ccccc5c4c3)cc2)cc1 ; c1ccc(-c2ccccc2-c2ccc(N(c3ccc4cc5c(cc4c3)oc3ccccc35)c3cccc4oc5ccccc5c34)cc2)cc1] 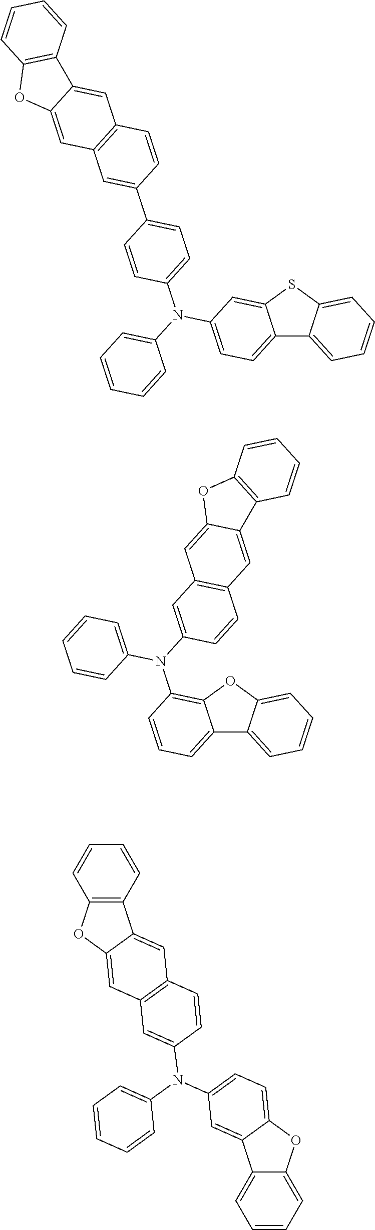 [SMILES: c1ccc(N(c2ccc(-c3ccc4cc5c(cc4c3)oc3ccccc35)cc2)c2ccc3c(c2)sc2ccccc23)cc1 ; c1ccc(N(c2ccc3cc4c(cc3c2)oc2ccccc24)c2cccc3c2oc2ccccc23)cc1 ; c1ccc(N(c2ccc3cc4c(cc3c2)oc2ccccc24)c2ccc3oc4ccccc4c3c2)cc1] 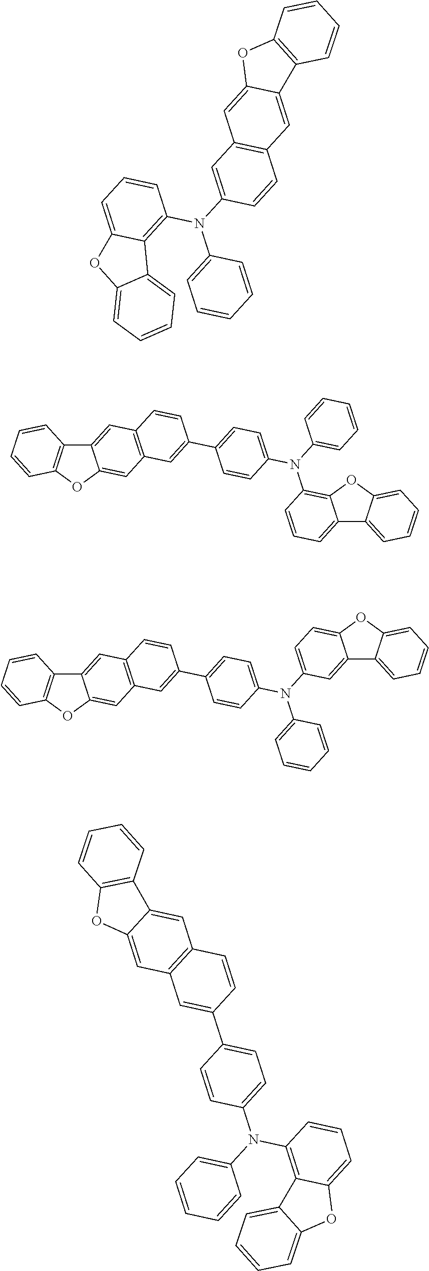 [SMILES: c1ccc(N(c2ccc3cc4c(cc3c2)oc2ccccc24)c2cccc3oc4ccccc4c23)cc1 ; c1ccc(N(c2ccc(-c3ccc4cc5c(cc4c3)oc3ccccc35)cc2)c2cccc3c2oc2ccccc23)cc1 ; c1ccc(N(c2ccc(-c3ccc4cc5c(cc4c3)oc3ccccc35)cc2)c2ccc3oc4ccccc4c3c2)cc1 ; c1ccc(N(c2ccc(-c3ccc4cc5c(cc4c3)oc3ccccc35)cc2)c2cccc3oc4ccccc4c23)cc1] 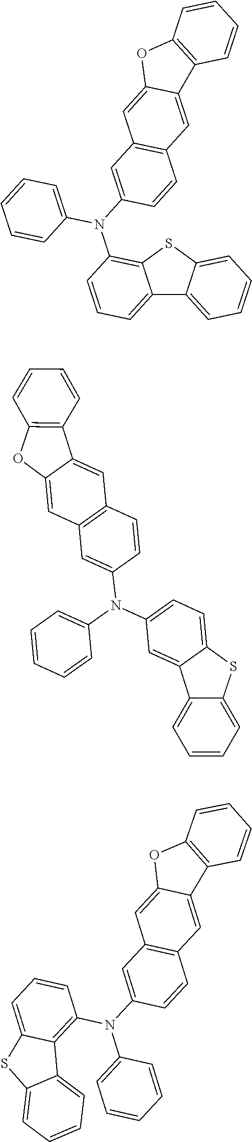 [SMILES: c1ccc(N(c2ccc3cc4c(cc3c2)oc2ccccc24)c2cccc3c2sc2ccccc23)cc1 ; c1ccc(N(c2ccc3cc4c(cc3c2)oc2ccccc24)c2ccc3sc4ccccc4c3c2)cc1 ; c1ccc(N(c2ccc3cc4c(cc3c2)oc2ccccc24)c2cccc3sc4ccccc4c23)cc1] 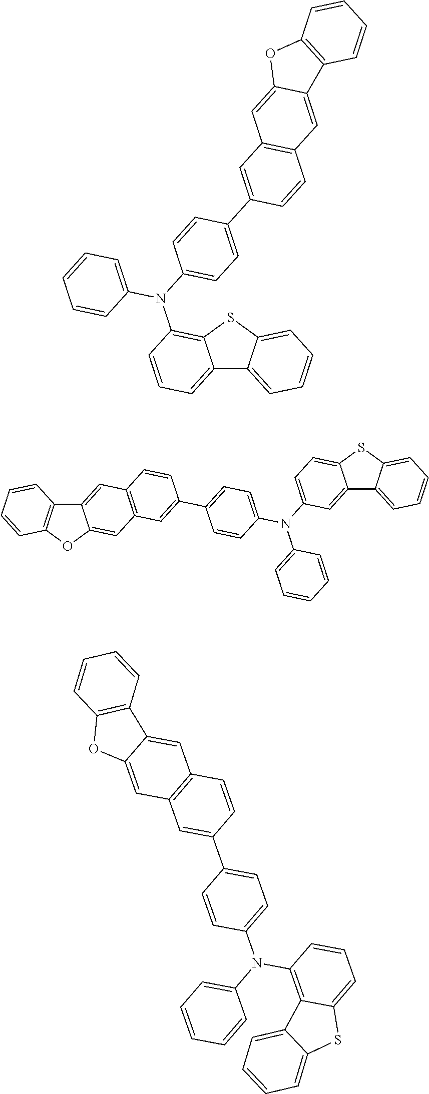 [SMILES: c1ccc(N(c2ccc(-c3ccc4cc5c(cc4c3)oc3ccccc35)cc2)c2cccc3c2sc2ccccc23)cc1 ; c1ccc(N(c2ccc(-c3ccc4cc5c(cc4c3)oc3ccccc35)cc2)c2ccc3sc4ccccc4c3c2)cc1 ; c1ccc(N(c2ccc(-c3ccc4cc5c(cc4c3)oc3ccccc35)cc2)c2cccc3sc4ccccc4c23)cc1] 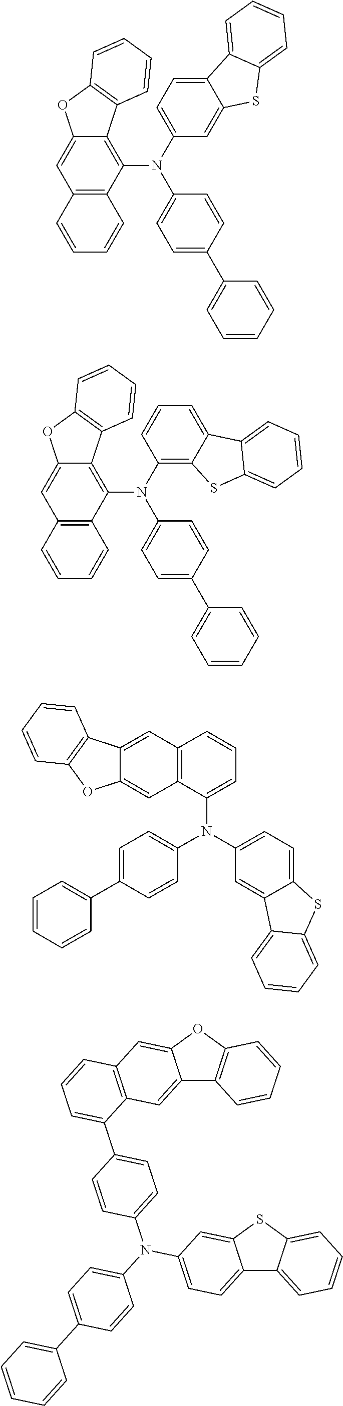 [SMILES: c1ccc(-c2ccc(N(c3ccc4c(c3)sc3ccccc34)c3c4ccccc4cc4oc5ccccc5c34)cc2)cc1 ; c1ccc(-c2ccc(N(c3cccc4c3sc3ccccc34)c3c4ccccc4cc4oc5ccccc5c34)cc2)cc1 ; c1ccc(-c2ccc(N(c3ccc4sc5ccccc5c4c3)c3cccc4cc5c(cc34)oc3ccccc35)cc2)cc1 ; c1ccc(-c2ccc(N(c3ccc(-c4cccc5cc6oc7ccccc7c6cc45)cc3)c3ccc4c(c3)sc3ccccc34)cc2)cc1] 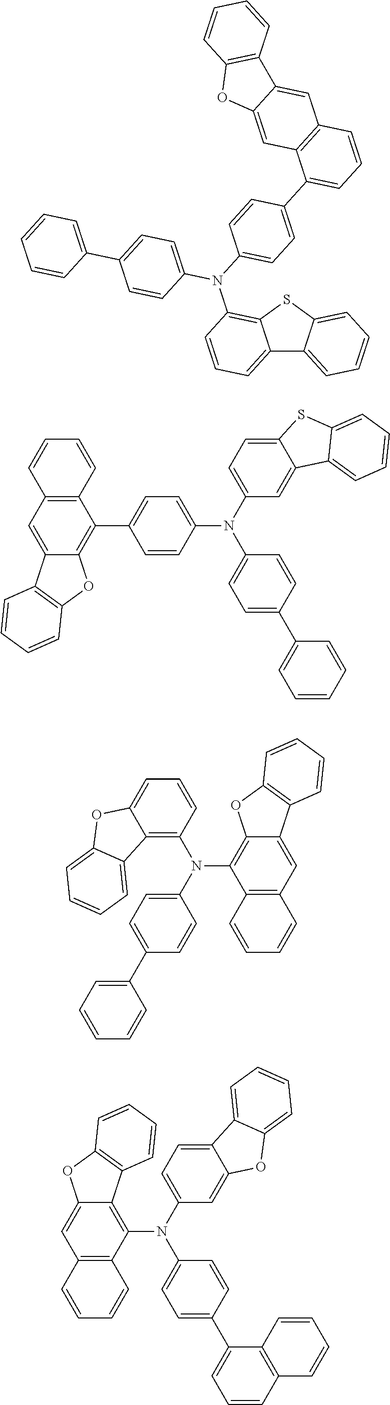 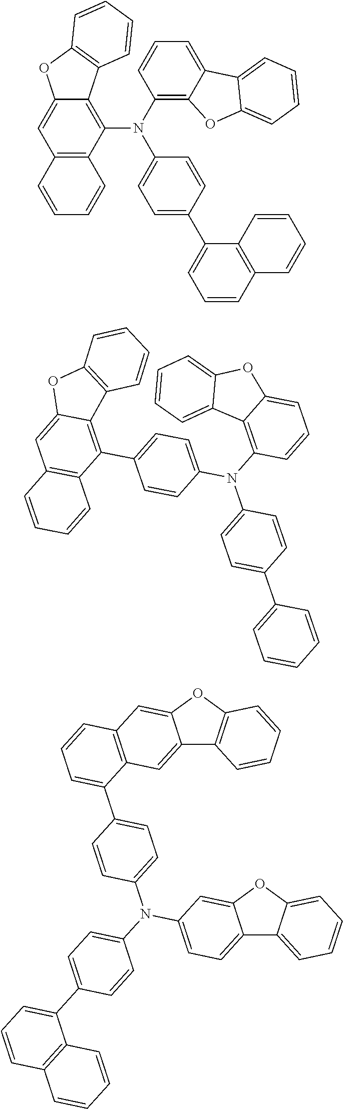 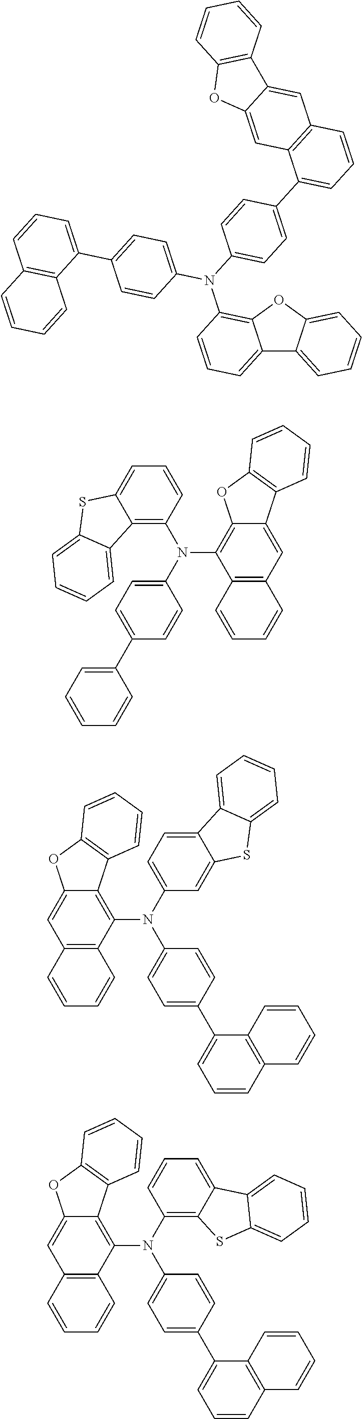 [SMILES: c1ccc2c(-c3ccc(N(c4ccc(-c5cccc6cc7c(cc56)oc5ccccc57)cc4)c4cccc5c4oc4ccccc45)cc3)cccc2c1 ; c1ccc(-c2ccc(N(c3c4ccccc4cc4c3oc3ccccc34)c3cccc4sc5ccccc5c34)cc2)cc1 ; c1ccc2c(-c3ccc(N(c4ccc5c(c4)sc4ccccc45)c4c5ccccc5cc5oc6ccccc6c45)cc3)cccc2c1 ; c1ccc2c(-c3ccc(N(c4cccc5c4sc4ccccc45)c4c5ccccc5cc5oc6ccccc6c45)cc3)cccc2c1] 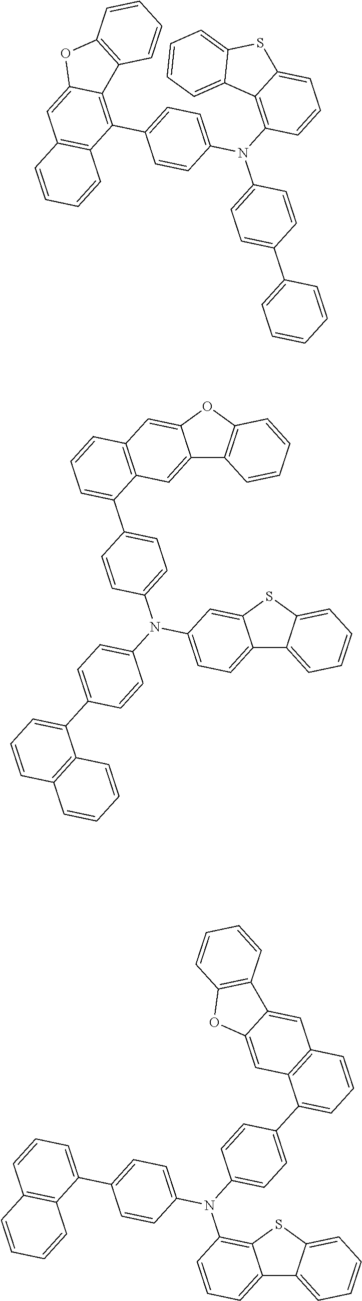 [SMILES: c1ccc(-c2ccc(N(c3ccc(-c4c5ccccc5cc5oc6ccccc6c45)cc3)c3cccc4sc5ccccc5c34)cc2)cc1 ; c1ccc2c(-c3ccc(N(c4ccc(-c5cccc6cc7oc8ccccc8c7cc56)cc4)c4ccc5c(c4)sc4ccccc45)cc3)cccc2c1 ; c1ccc2c(-c3ccc(N(c4ccc(-c5cccc6cc7c(cc56)oc5ccccc57)cc4)c4cccc5c4sc4ccccc45)cc3)cccc2c1] 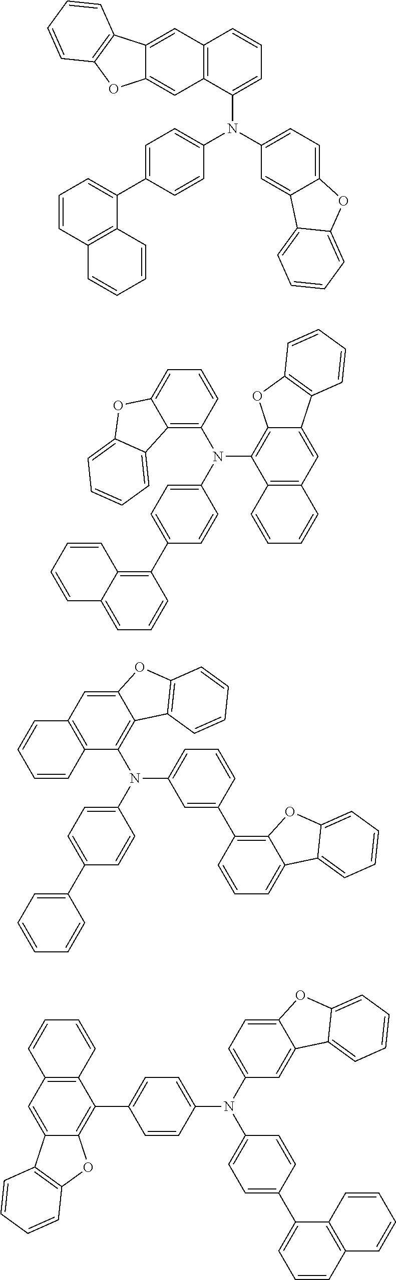 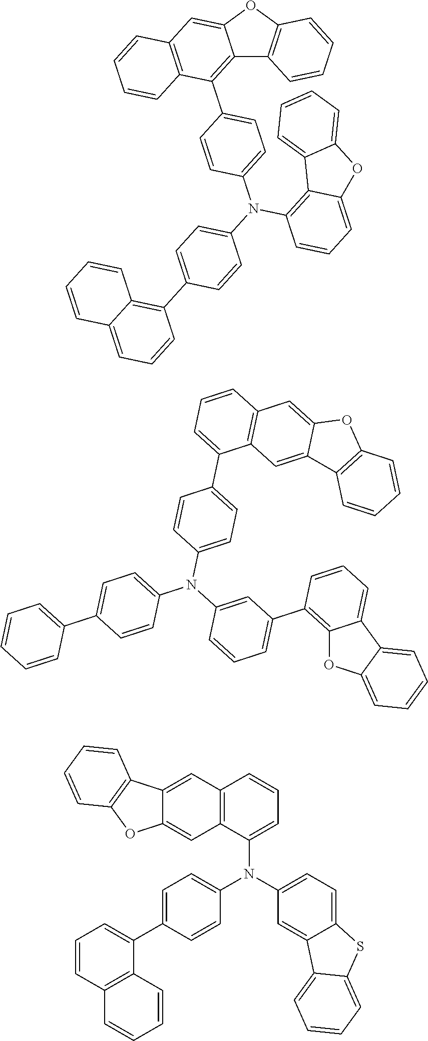 [SMILES: c1ccc2c(-c3ccc(N(c4ccc(-c5c6ccccc6cc6oc7ccccc7c56)cc4)c4cccc5oc6ccccc6c45)cc3)cccc2c1 ; c1ccc(-c2ccc(N(c3ccc(-c4cccc5cc6oc7ccccc7c6cc45)cc3)c3cccc(-c4cccc5c4oc4ccccc45)c3)cc2)cc1 ; c1ccc2c(-c3ccc(N(c4ccc5sc6ccccc6c5c4)c4cccc5cc6c(cc45)oc4ccccc46)cc3)cccc2c1] 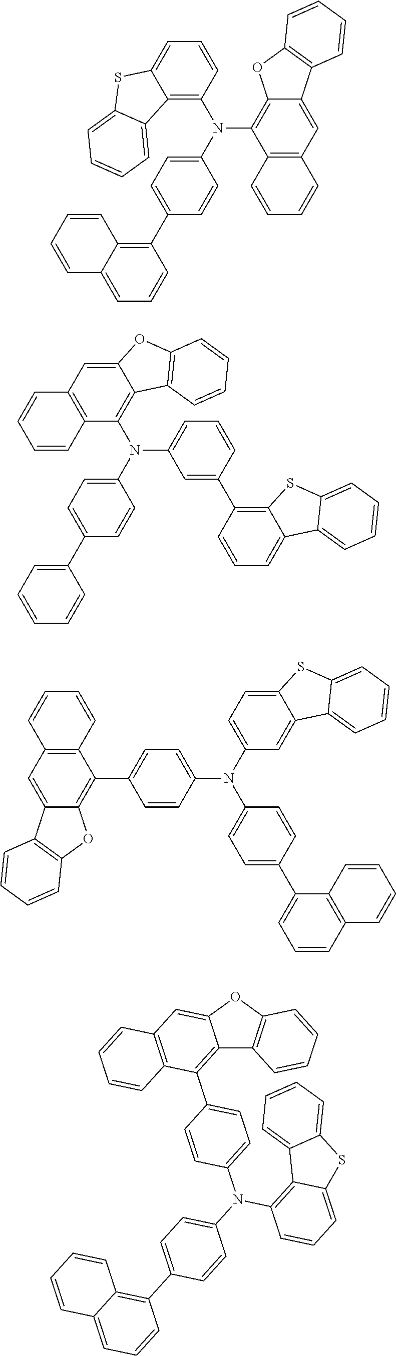 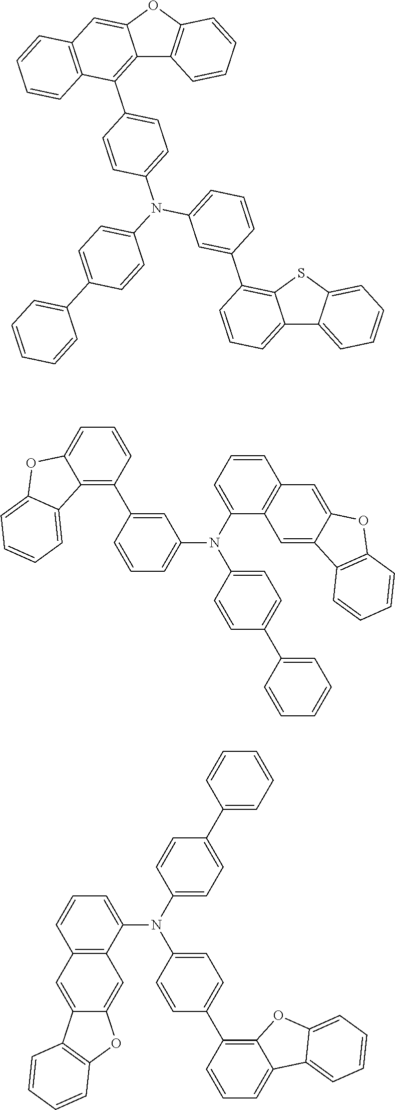 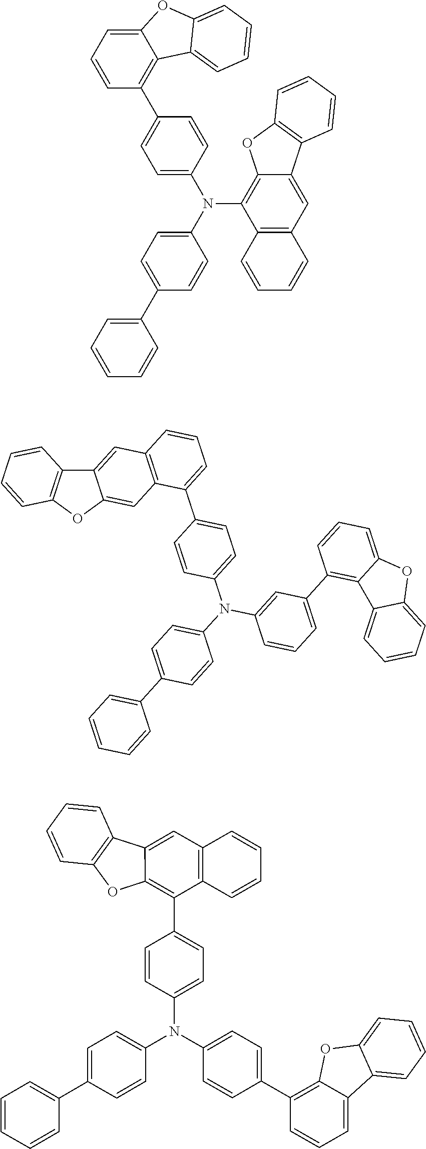 [SMILES: c1ccc(-c2ccc(N(c3ccc(-c4cccc5oc6ccccc6c45)cc3)c3c4ccccc4cc4c3oc3ccccc34)cc2)cc1 ; c1ccc(-c2ccc(N(c3ccc(-c4cccc5cc6c(cc45)oc4ccccc46)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4c5ccccc5cc5c4oc4ccccc45)cc3)cc2)cc1] 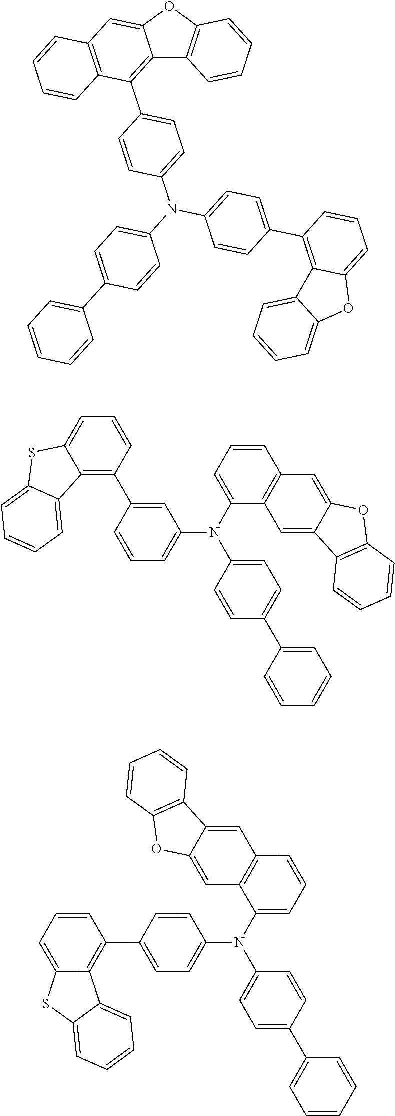 [SMILES: c1ccc(-c2ccc(N(c3ccc(-c4cccc5oc6ccccc6c45)cc3)c3ccc(-c4c5ccccc5cc5oc6ccccc6c45)cc3)cc2)cc1 ; c1ccc(-c2ccc(N(c3cccc(-c4cccc5sc6ccccc6c45)c3)c3cccc4cc5oc6ccccc6c5cc34)cc2)cc1 ; c1ccc(-c2ccc(N(c3ccc(-c4cccc5sc6ccccc6c45)cc3)c3cccc4cc5c(cc34)oc3ccccc35)cc2)cc1] 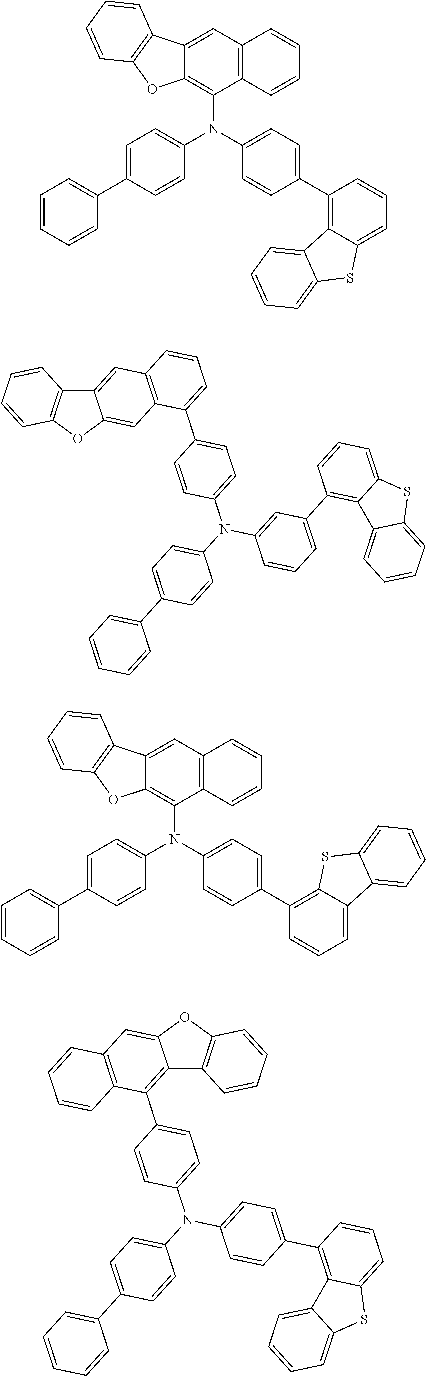 [SMILES: c1ccc(-c2ccc(N(c3ccc(-c4cccc5sc6ccccc6c45)cc3)c3c4ccccc4cc4c3oc3ccccc34)cc2)cc1 ; c1ccc(-c2ccc(N(c3ccc(-c4cccc5cc6c(cc45)oc4ccccc46)cc3)c3cccc(-c4cccc5sc6ccccc6c45)c3)cc2)cc1 ; c1ccc(-c2ccc(N(c3ccc(-c4cccc5c4sc4ccccc45)cc3)c3c4ccccc4cc4c3oc3ccccc34)cc2)cc1 ; c1ccc(-c2ccc(N(c3ccc(-c4c5ccccc5cc5oc6ccccc6c45)cc3)c3ccc(-c4cccc5sc6ccccc6c45)cc3)cc2)cc1] 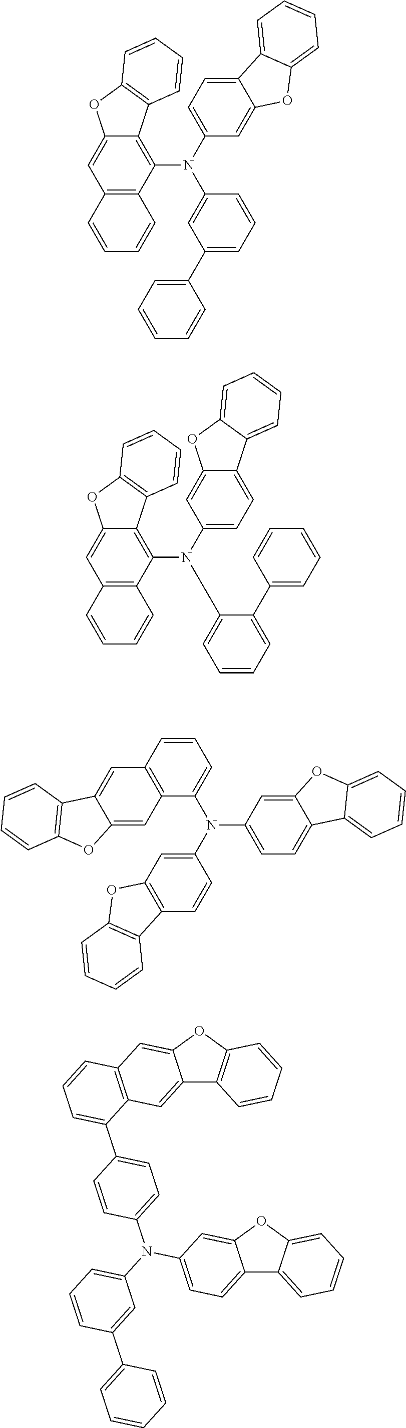 [SMILES: c1ccc(-c2cccc(N(c3ccc4c(c3)oc3ccccc34)c3c4ccccc4cc4oc5ccccc5c34)c2)cc1 ; c1ccc(-c2ccccc2CN(c2ccc3c(c2)oc2ccccc23)c2c3ccccc3cc3oc4ccccc4c23)cc1 ; c1cc(N(c2ccc3c(c2)oc2ccccc23)c2ccc3c(c2)oc2ccccc23)c2cc3oc4ccccc4c3cc2c1 ; c1ccc(-c2cccc(N(c3ccc(-c4cccc5cc6oc7ccccc7c6cc45)cc3)c3ccc4c(c3)oc3ccccc34)c2)cc1] 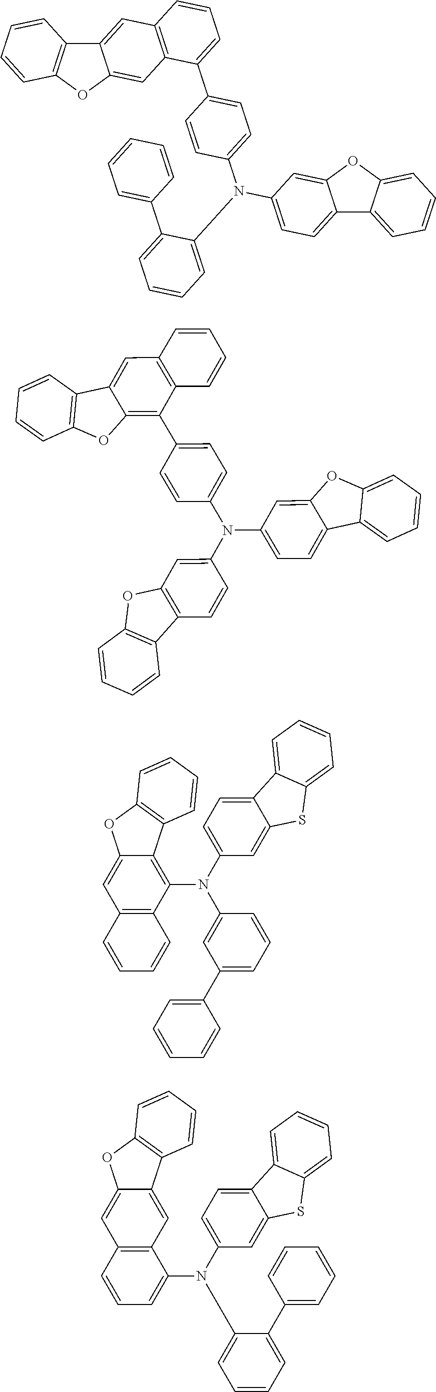 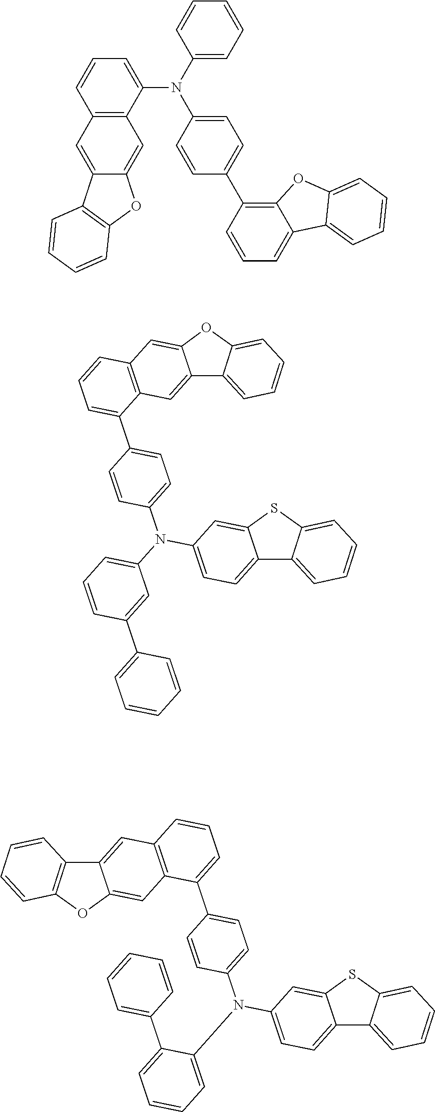 [SMILES: c1ccc(N(c2ccc(-c3cccc4c3oc3ccccc34)cc2)c2cccc3cc4c(cc23)oc2ccccc24)cc1 ; c1ccc(-c2cccc(N(c3ccc(-c4cccc5cc6oc7ccccc7c6cc45)cc3)c3ccc4c(c3)sc3ccccc34)c2)cc1 ; c1ccc(-c2ccccc2CN(c2ccc(-c3cccc4cc5c(cc34)oc3ccccc35)cc2)c2ccc3c(c2)sc2ccccc23)cc1] 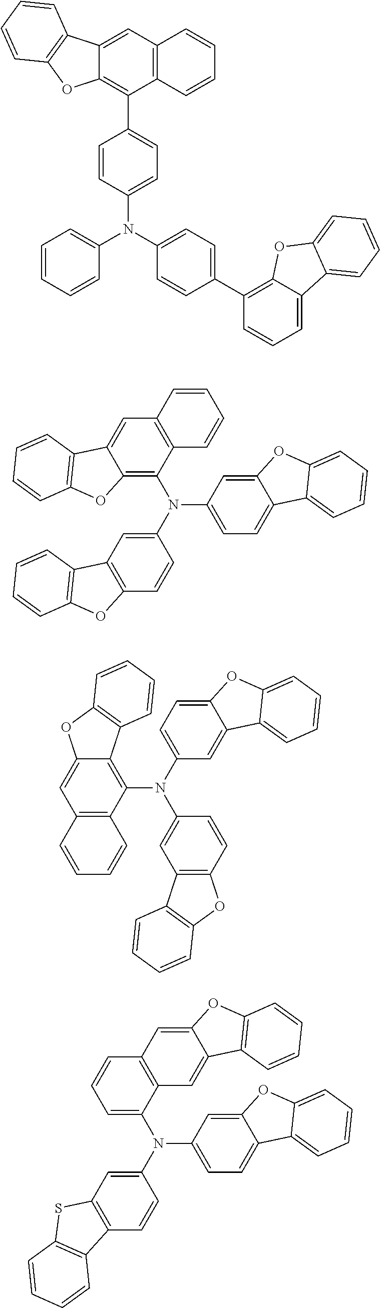 [SMILES: c1ccc(N(c2ccc(-c3cccc4c3oc3ccccc34)cc2)c2ccc(-c3c4ccccc4cc4c3oc3ccccc34)cc2)cc1 ; c1ccc2c(N(c3ccc4c(c3)oc3ccccc34)c3ccc4oc5ccccc5c4c3)c3oc4ccccc4c3cc2c1 ; c1ccc2c(N(c3ccc4oc5ccccc5c4c3)c3ccc4oc5ccccc5c4c3)c3c(cc2c1)oc1ccccc13 ; c1cc(N(c2ccc3c(c2)oc2ccccc23)c2ccc3c(c2)sc2ccccc23)c2cc3c(cc2c1)oc1ccccc13] 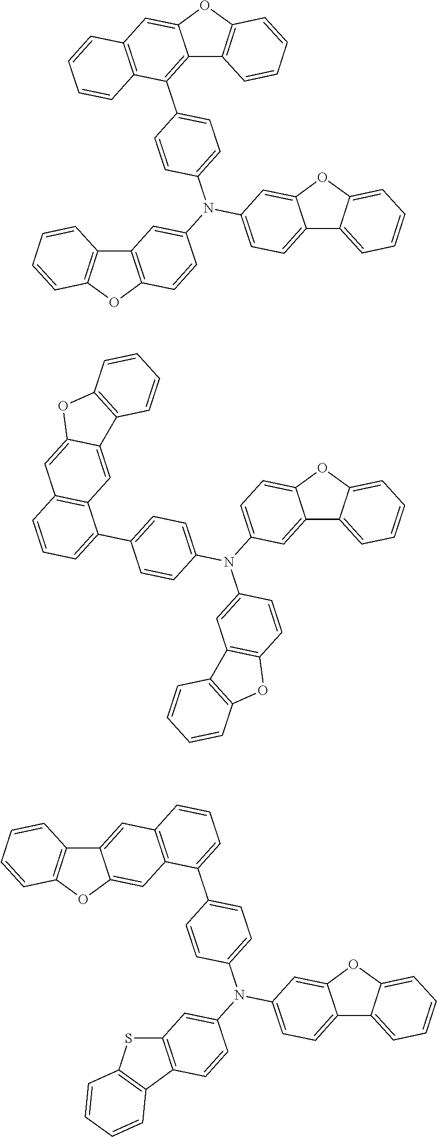 [SMILES: c1ccc2c(-c3ccc(N(c4ccc5c(c4)oc4ccccc45)c4ccc5oc6ccccc6c5c4)cc3)c3c(cc2c1)oc1ccccc13 ; c1cc(-c2ccc(N(c3ccc4oc5ccccc5c4c3)c3ccc4oc5ccccc5c4c3)cc2)c2cc3c(cc2c1)oc1ccccc13 ; c1cc(-c2ccc(N(c3ccc4c(c3)oc3ccccc34)c3ccc4c(c3)sc3ccccc34)cc2)c2cc3oc4ccccc4c3cc2c1] 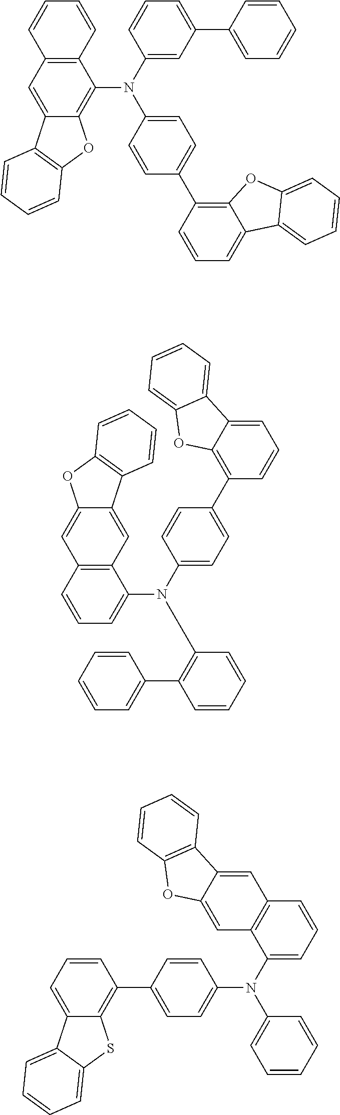 [SMILES: c1ccc(-c2cccc(N(c3ccc(-c4cccc5c4oc4ccccc45)cc3)c3c4ccccc4cc4c3oc3ccccc34)c2)cc1 ; c1ccc(-c2ccccc2CN(c2ccc(-c3cccc4c3oc3ccccc34)cc2)c2cccc3cc4oc5ccccc5c4cc23)cc1 ; c1ccc(N(c2ccc(-c3cccc4c3sc3ccccc34)cc2)c2cccc3cc4c(cc23)oc2ccccc24)cc1] 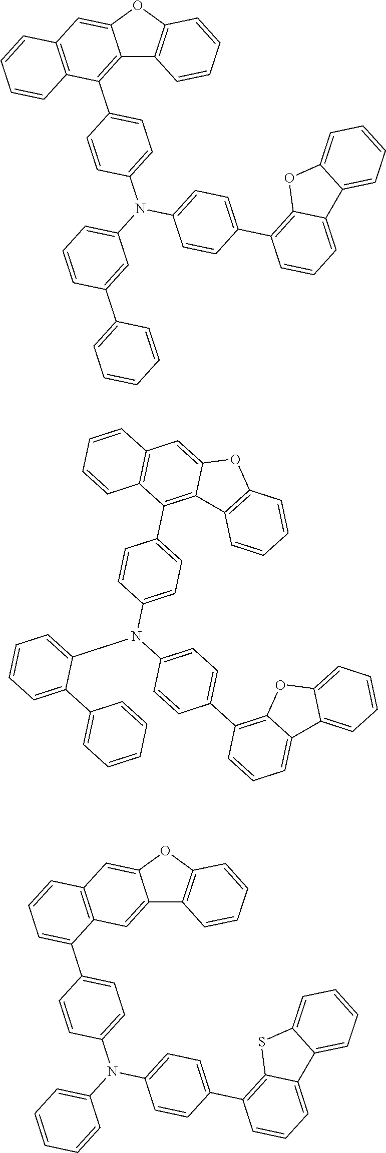 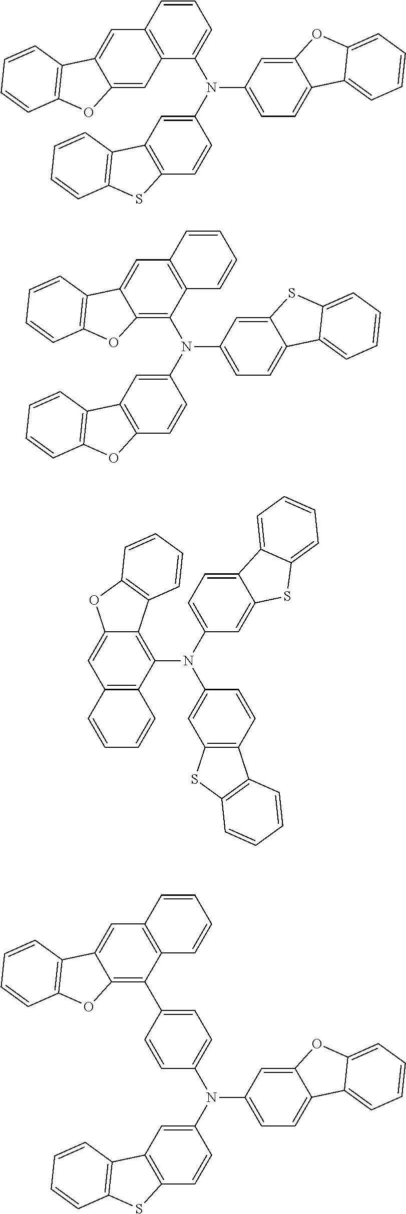 [SMILES: c1cc(N(c2ccc3c(c2)oc2ccccc23)c2ccc3sc4ccccc4c3c2)c2cc3oc4ccccc4c3cc2c1 ; c1ccc2c(N(c3ccc4c(c3)sc3ccccc34)c3ccc4oc5ccccc5c4c3)c3oc4ccccc4c3cc2c1 ; c1ccc2c(N(c3ccc4c(c3)sc3ccccc34)c3ccc4c(c3)sc3ccccc34)c3c(cc2c1)oc1ccccc13 ; c1ccc2c(-c3ccc(N(c4ccc5c(c4)oc4ccccc45)c4ccc5sc6ccccc6c5c4)cc3)c3oc4ccccc4c3cc2c1] 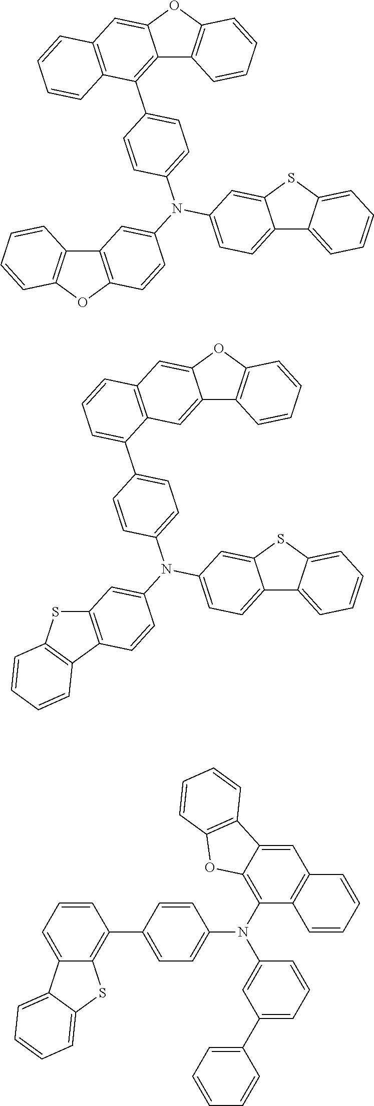 [SMILES: c1ccc2c(-c3ccc(N(c4ccc5c(c4)sc4ccccc45)c4ccc5oc6ccccc6c5c4)cc3)c3c(cc2c1)oc1ccccc13 ; c1cc(-c2ccc(N(c3ccc4c(c3)sc3ccccc34)c3ccc4c(c3)sc3ccccc34)cc2)c2cc3c(cc2c1)oc1ccccc13 ; c1ccc(-c2cccc(N(c3ccc(-c4cccc5c4sc4ccccc45)cc3)c3c4ccccc4cc4c3oc3ccccc34)c2)cc1] 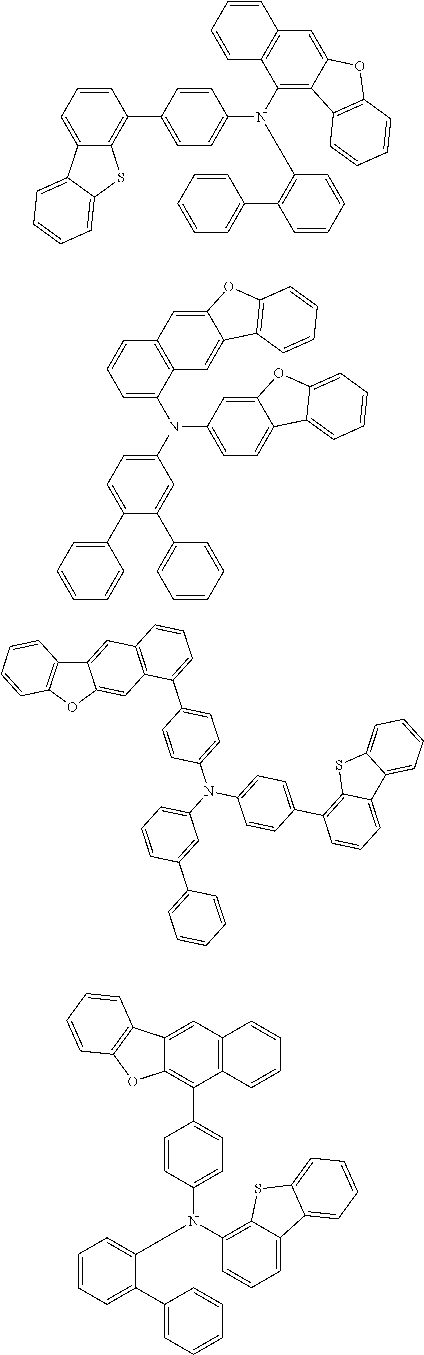 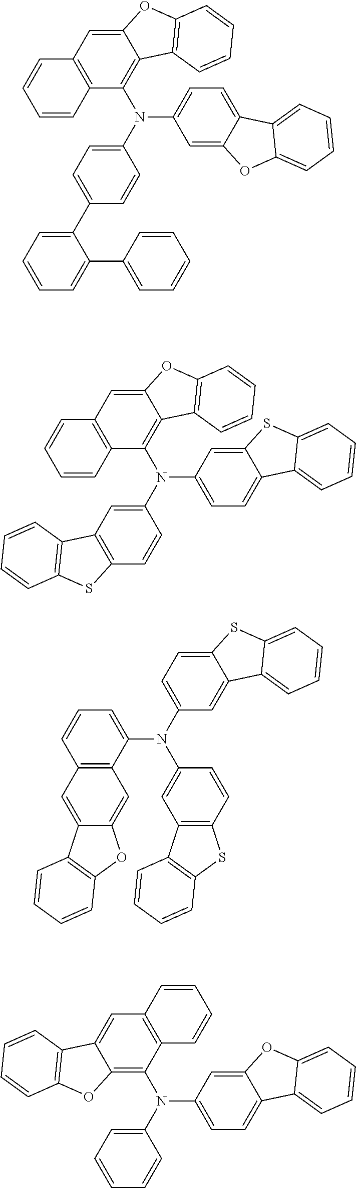 [SMILES: c1ccc(-c2ccccc2-c2ccc(N(c3ccc4c(c3)oc3ccccc34)c3c4ccccc4cc4oc5ccccc5c34)cc2)cc1 ; c1ccc2c(N(c3ccc4c(c3)sc3ccccc34)c3ccc4sc5ccccc5c4c3)c3c(cc2c1)oc1ccccc13 ; c1cc(N(c2ccc3sc4ccccc4c3c2)c2ccc3sc4ccccc4c3c2)c2cc3oc4ccccc4c3cc2c1 ; c1ccc(N(c2ccc3c(c2)oc2ccccc23)c2c3ccccc3cc3c2oc2ccccc23)cc1] 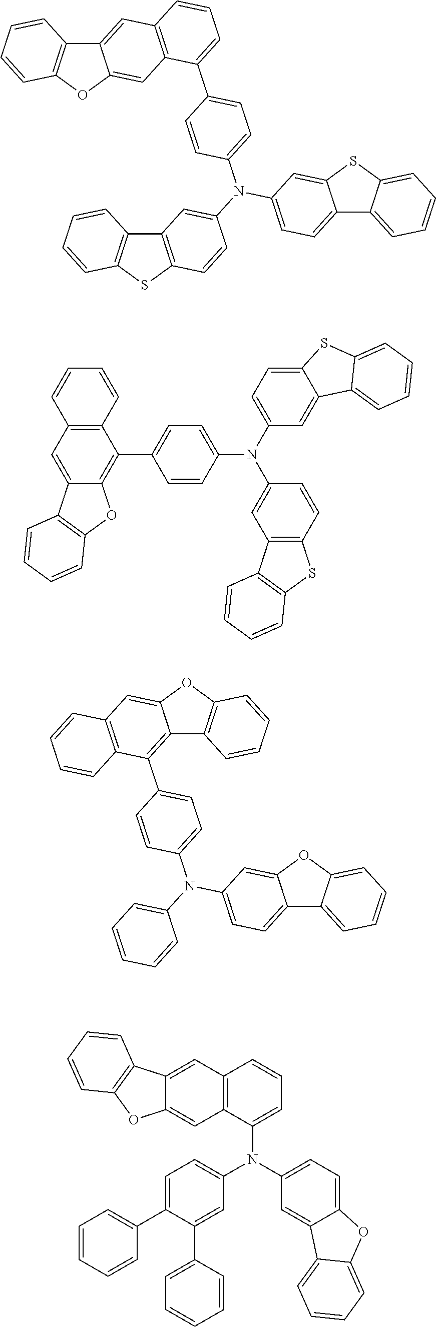 [SMILES: c1cc(-c2ccc(N(c3ccc4c(c3)sc3ccccc34)c3ccc4sc5ccccc5c4c3)cc2)c2cc3oc4ccccc4c3cc2c1 ; c1ccc2c(-c3ccc(N(c4ccc5sc6ccccc6c5c4)c4ccc5sc6ccccc6c5c4)cc3)c3oc4ccccc4c3cc2c1 ; c1ccc(N(c2ccc(-c3c4ccccc4cc4oc5ccccc5c34)cc2)c2ccc3c(c2)oc2ccccc23)cc1 ; c1ccc(-c2ccc(N(c3ccc4oc5ccccc5c4c3)c3cccc4cc5c(cc34)oc3ccccc35)cc2-c2ccccc2)cc1] 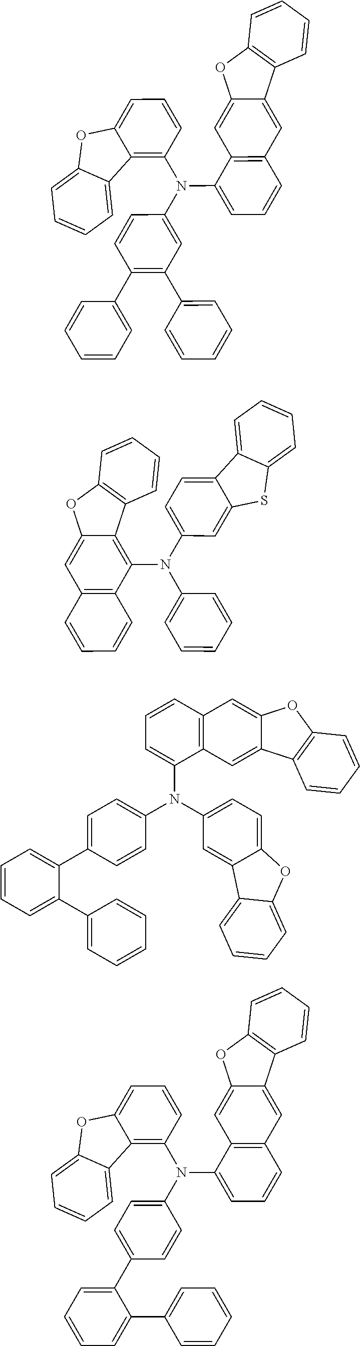 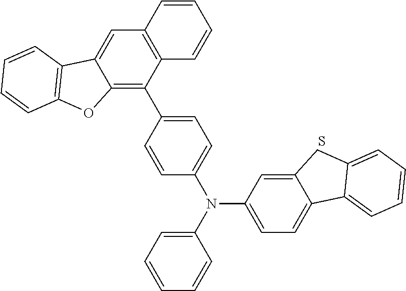 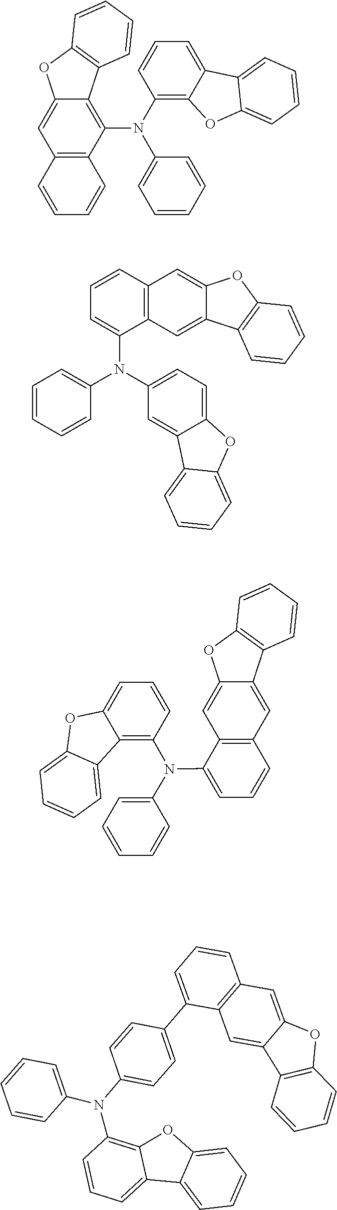 [SMILES: c1ccc(N(c2cccc3c2oc2ccccc23)c2c3ccccc3cc3oc4ccccc4c23)cc1 ; c1ccc(N(c2ccc3oc4ccccc4c3c2)c2cccc3cc4oc5ccccc5c4cc23)cc1 ; c1ccc(N(c2cccc3cc4c(cc23)oc2ccccc24)c2cccc3oc4ccccc4c23)cc1 ; c1ccc(N(c2ccc(-c3cccc4cc5oc6ccccc6c5cc34)cc2)c2cccc3c2oc2ccccc23)cc1] 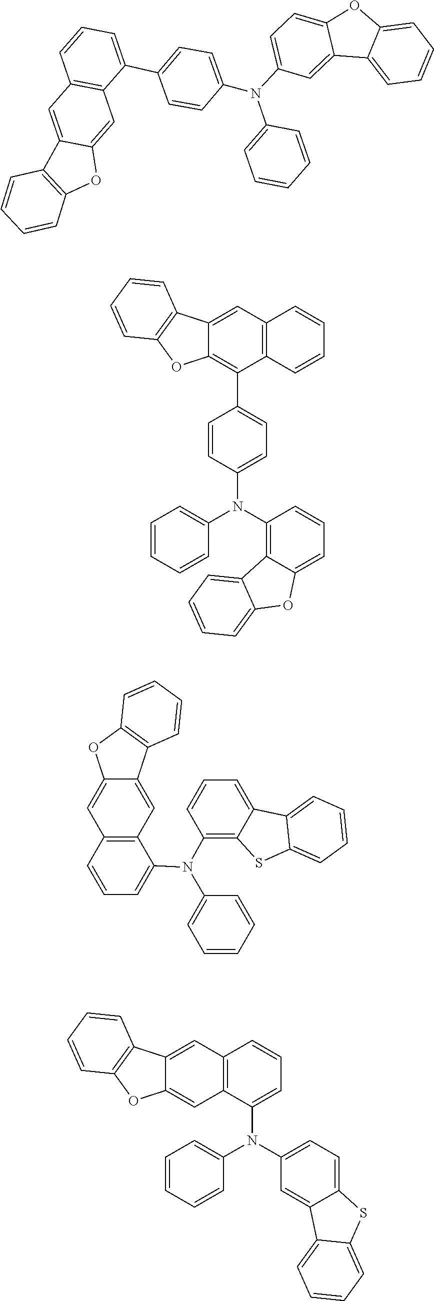 [SMILES: c1ccc(N(c2ccc(-c3cccc4cc5c(cc34)oc3ccccc35)cc2)c2ccc3oc4ccccc4c3c2)cc1 ; c1ccc(N(c2ccc(-c3c4ccccc4cc4c3oc3ccccc34)cc2)c2cccc3oc4ccccc4c23)cc1 ; c1ccc(N(c2cccc3cc4oc5ccccc5c4cc23)c2cccc3c2sc2ccccc23)cc1 ; c1ccc(N(c2ccc3sc4ccccc4c3c2)c2cccc3cc4c(cc23)oc2ccccc24)cc1] 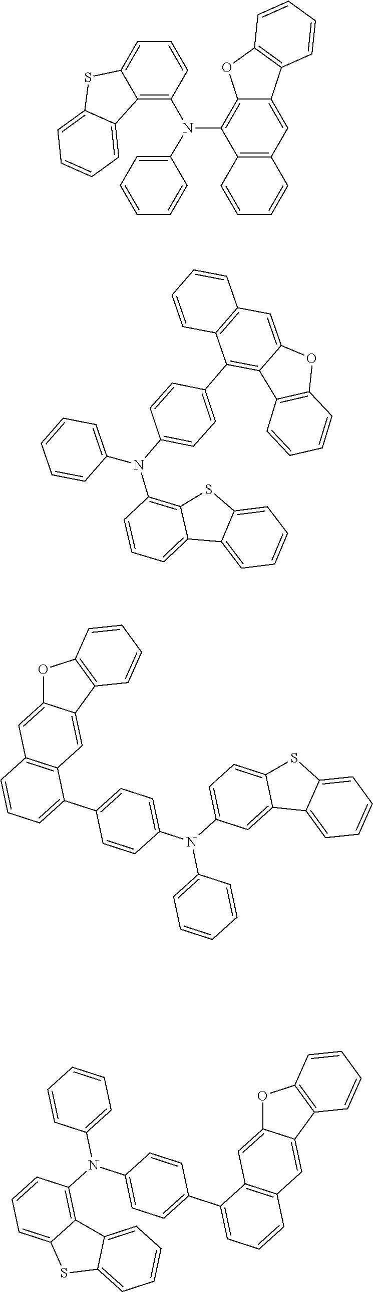 [SMILES: c1ccc(N(c2c3ccccc3cc3c2oc2ccccc23)c2cccc3sc4ccccc4c23)cc1 ; c1ccc(N(c2ccc(-c3c4ccccc4cc4oc5ccccc5c34)cc2)c2cccc3c2sc2ccccc23)cc1 ; c1ccc(N(c2ccc(-c3cccc4cc5oc6ccccc6c5cc34)cc2)c2ccc3sc4ccccc4c3c2)cc1 ; c1ccc(N(c2ccc(-c3cccc4cc5c(cc34)oc3ccccc35)cc2)c2cccc3sc4ccccc4c23)cc1] 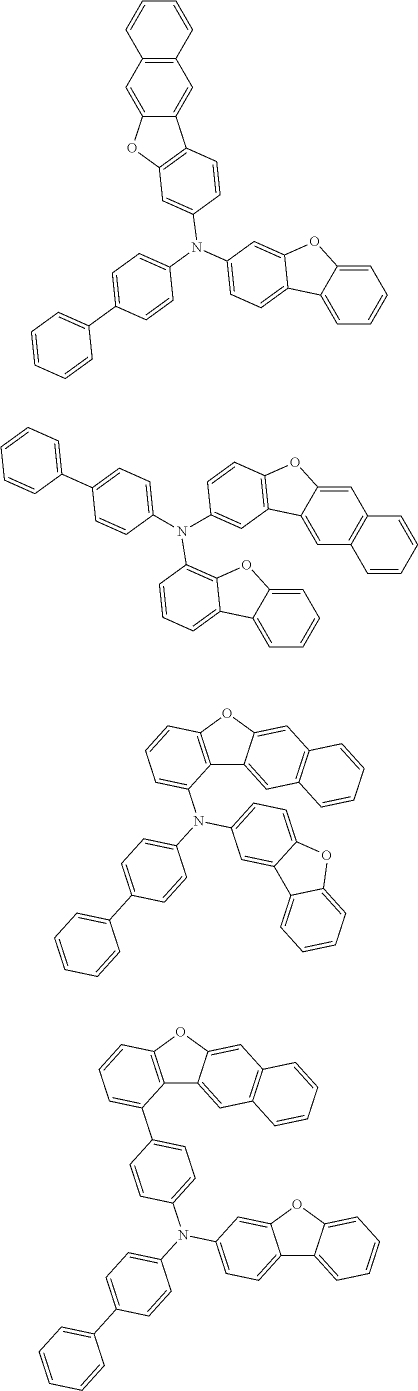 [SMILES: c1ccc(-c2ccc(N(c3ccc4c(c3)oc3ccccc34)c3ccc4c(c3)oc3cc5ccccc5cc34)cc2)cc1 ; c1ccc(-c2ccc(N(c3ccc4oc5cc6ccccc6cc5c4c3)c3cccc4c3oc3ccccc34)cc2)cc1 ; c1ccc(-c2ccc(N(c3ccc4oc5ccccc5c4c3)c3cccc4oc5cc6ccccc6cc5c34)cc2)cc1 ; c1ccc(-c2ccc(N(c3ccc(-c4cccc5oc6cc7ccccc7cc6c45)cc3)c3ccc4c(c3)oc3ccccc34)cc2)cc1] 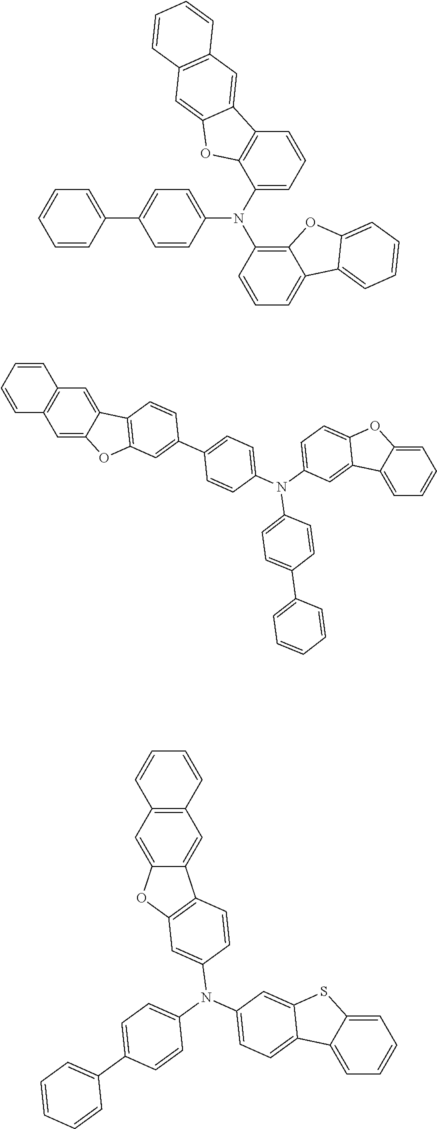 [SMILES: c1ccc(-c2ccc(N(c3cccc4c3oc3ccccc34)c3cccc4c3oc3cc5ccccc5cc34)cc2)cc1 ; c1ccc(-c2ccc(N(c3ccc(-c4ccc5c(c4)oc4cc6ccccc6cc45)cc3)c3ccc4oc5ccccc5c4c3)cc2)cc1 ; c1ccc(-c2ccc(N(c3ccc4c(c3)oc3cc5ccccc5cc34)c3ccc4c(c3)sc3ccccc34)cc2)cc1] 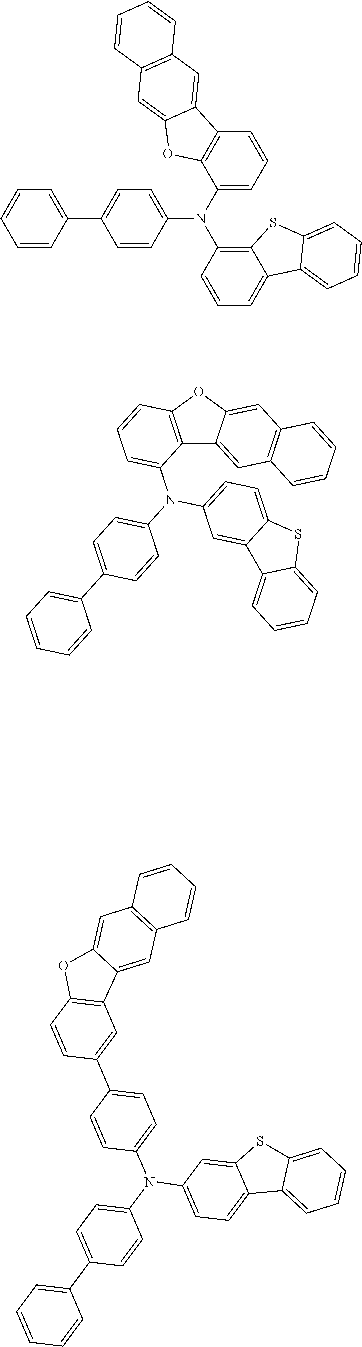 [SMILES: c1ccc(-c2ccc(N(c3cccc4c3oc3cc5ccccc5cc34)c3cccc4c3sc3ccccc34)cc2)cc1 ; c1ccc(-c2ccc(N(c3ccc4sc5ccccc5c4c3)c3cccc4oc5cc6ccccc6cc5c34)cc2)cc1 ; c1ccc(-c2ccc(N(c3ccc(-c4ccc5oc6cc7ccccc7cc6c5c4)cc3)c3ccc4c(c3)sc3ccccc34)cc2)cc1] 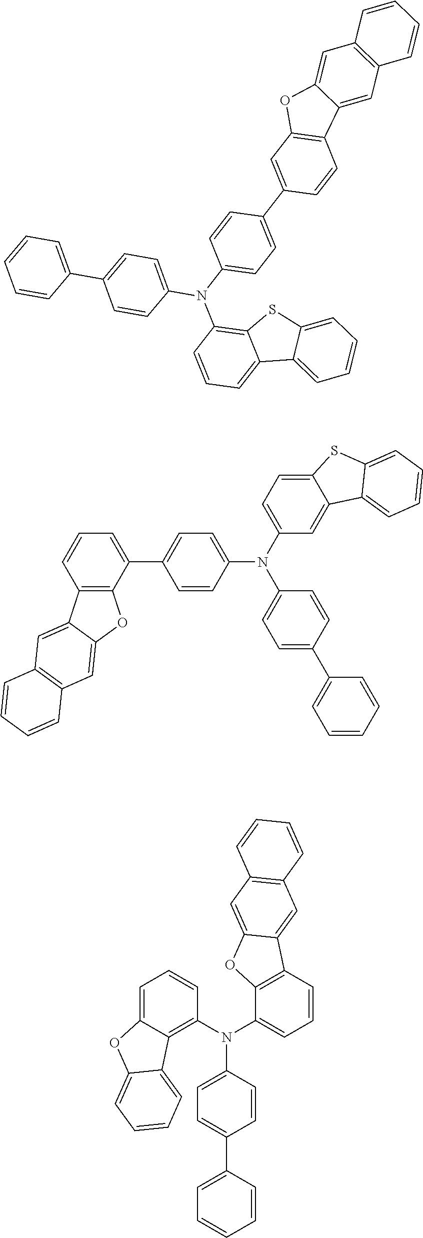 [SMILES: c1ccc(-c2ccc(N(c3ccc(-c4ccc5c(c4)oc4cc6ccccc6cc45)cc3)c3cccc4c3sc3ccccc34)cc2)cc1 ; c1ccc(-c2ccc(N(c3ccc(-c4cccc5c4oc4cc6ccccc6cc45)cc3)c3ccc4sc5ccccc5c4c3)cc2)cc1 ; c1ccc(-c2ccc(N(c3cccc4c3oc3cc5ccccc5cc34)c3cccc4oc5ccccc5c34)cc2)cc1] 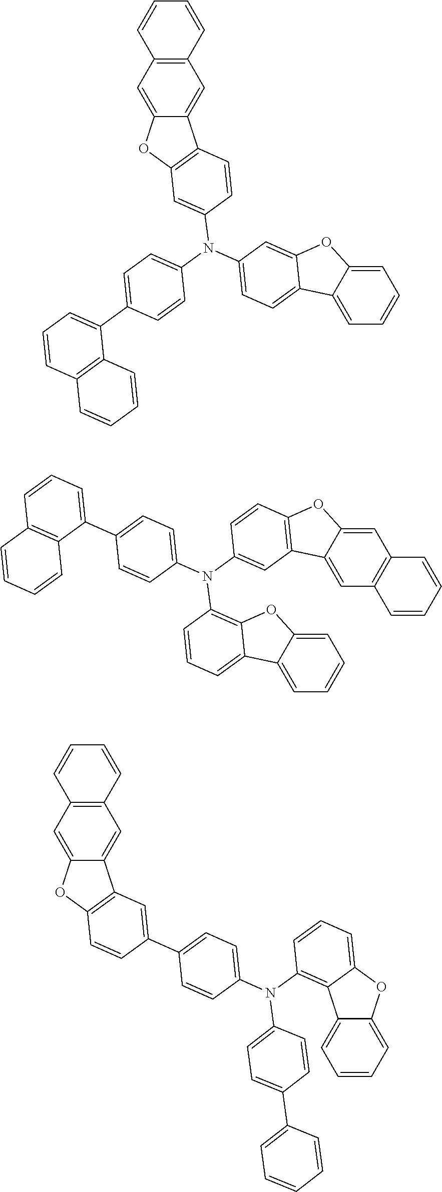 [SMILES: c1ccc2cc3c(cc2c1)oc1cc(N(c2ccc(-c4cccc5ccccc45)cc2)c2ccc4c(c2)oc2ccccc24)ccc13 ; c1ccc2cc3c(cc2c1)oc1ccc(N(c2ccc(-c4cccc5ccccc45)cc2)c2cccc4c2oc2ccccc24)cc13 ; c1ccc(-c2ccc(N(c3ccc(-c4ccc5oc6cc7ccccc7cc6c5c4)cc3)c3cccc4oc5ccccc5c34)cc2)cc1] 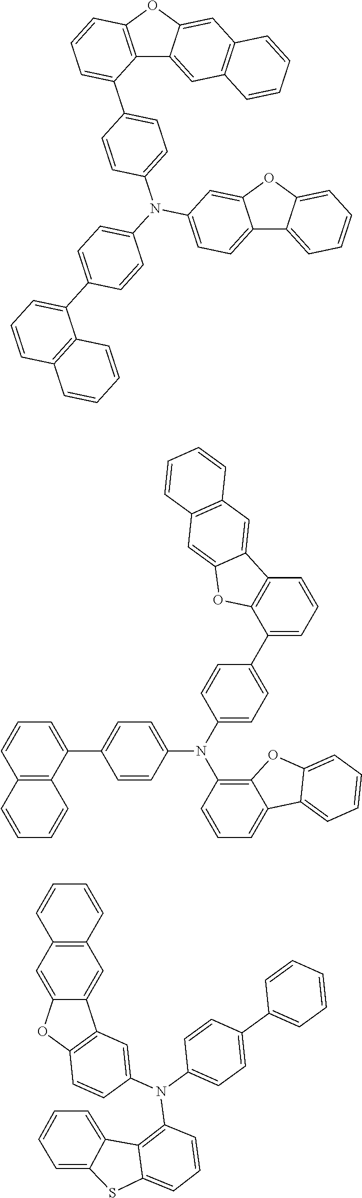 [SMILES: c1ccc2cc3c(cc2c1)oc1cccc(-c2ccc(N(c4ccc(-c5cccc6ccccc56)cc4)c4ccc5c(c4)oc4ccccc45)cc2)c13 ; c1ccc2cc3c(cc2c1)oc1c(-c2ccc(N(c4ccc(-c5cccc6ccccc56)cc4)c4cccc5c4oc4ccccc45)cc2)cccc13 ; c1ccc(-c2ccc(N(c3ccc4oc5cc6ccccc6cc5c4c3)c3cccc4sc5ccccc5c34)cc2)cc1] 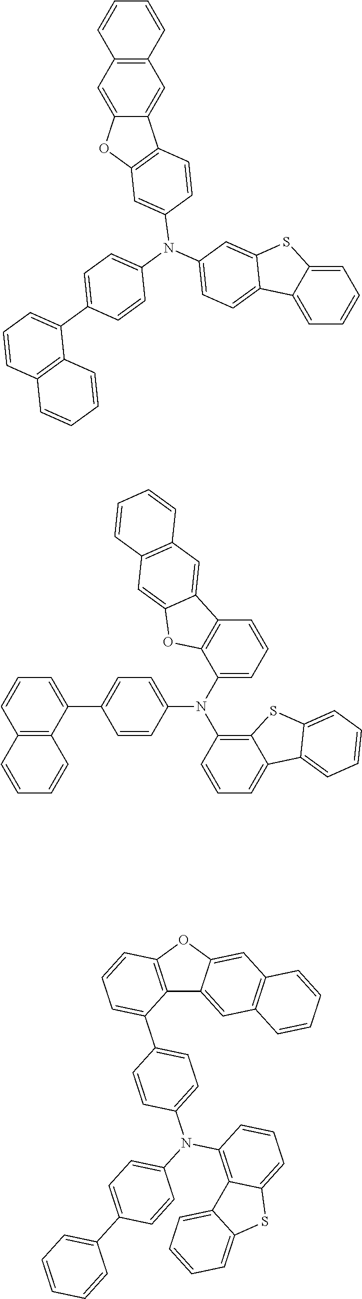 [SMILES: c1ccc2cc3c(cc2c1)oc1cc(N(c2ccc(-c4cccc5ccccc45)cc2)c2ccc4c(c2)sc2ccccc24)ccc13 ; c1ccc2cc3c(cc2c1)oc1c(N(c2ccc(-c4cccc5ccccc45)cc2)c2cccc4c2sc2ccccc24)cccc13 ; c1ccc(-c2ccc(N(c3ccc(-c4cccc5oc6cc7ccccc7cc6c45)cc3)c3cccc4sc5ccccc5c34)cc2)cc1] 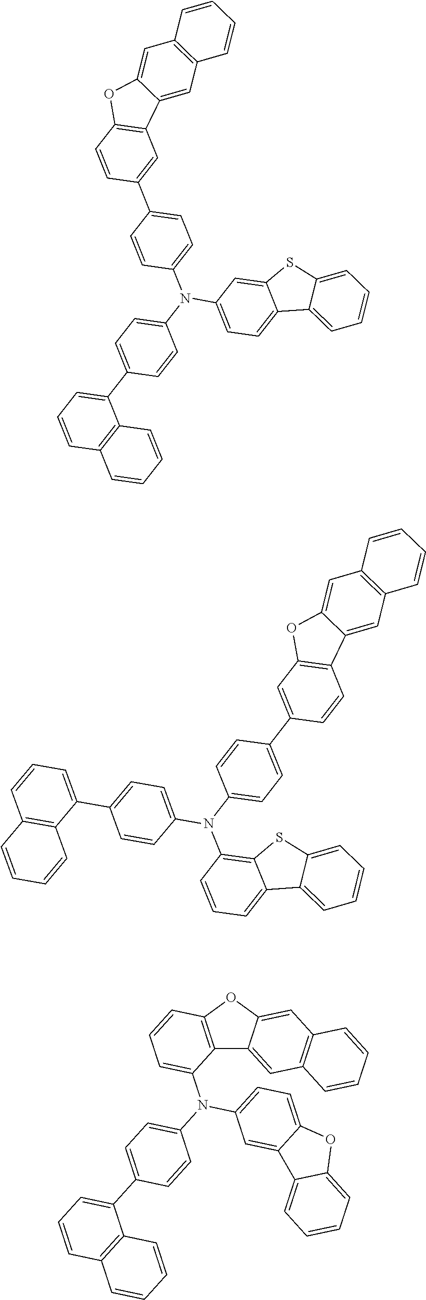 [SMILES: c1ccc2cc3c(cc2c1)oc1ccc(-c2ccc(N(c4ccc(-c5cccc6ccccc56)cc4)c4ccc5c(c4)sc4ccccc45)cc2)cc13 ; c1ccc2cc3c(cc2c1)oc1cc(-c2ccc(N(c4ccc(-c5cccc6ccccc56)cc4)c4cccc5c4sc4ccccc45)cc2)ccc13 ; c1ccc2cc3c(cc2c1)oc1cccc(N(c2ccc(-c4cccc5ccccc45)cc2)c2ccc4oc5ccccc5c4c2)c13] 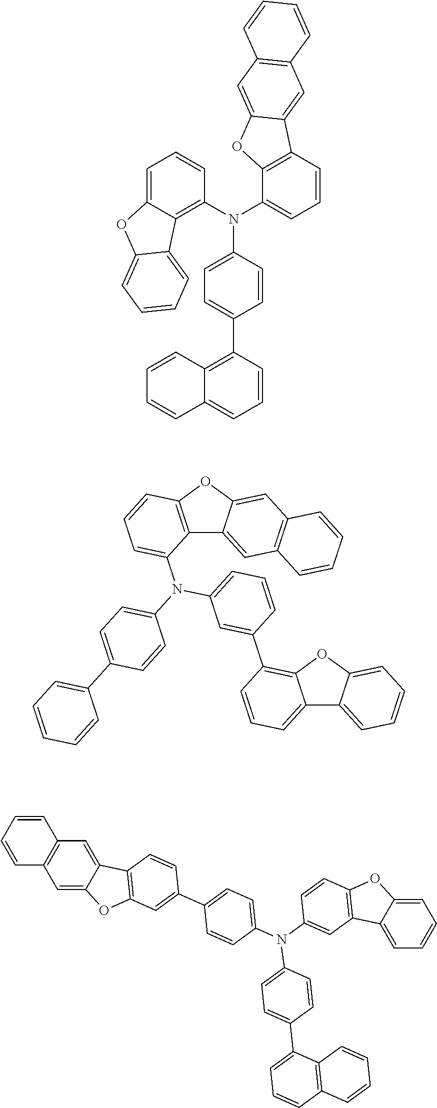 [SMILES: c1ccc2cc3c(cc2c1)oc1c(N(c2ccc(-c4cccc5ccccc45)cc2)c2cccc4oc5ccccc5c24)cccc13 ; c1ccc(-c2ccc(N(c3cccc(-c4cccc5c4oc4ccccc45)c3)c3cccc4oc5cc6ccccc6cc5c34)cc2)cc1 ; c1ccc2cc3c(cc2c1)oc1cc(-c2ccc(N(c4ccc(-c5cccc6ccccc56)cc4)c4ccc5oc6ccccc6c5c4)cc2)ccc13] 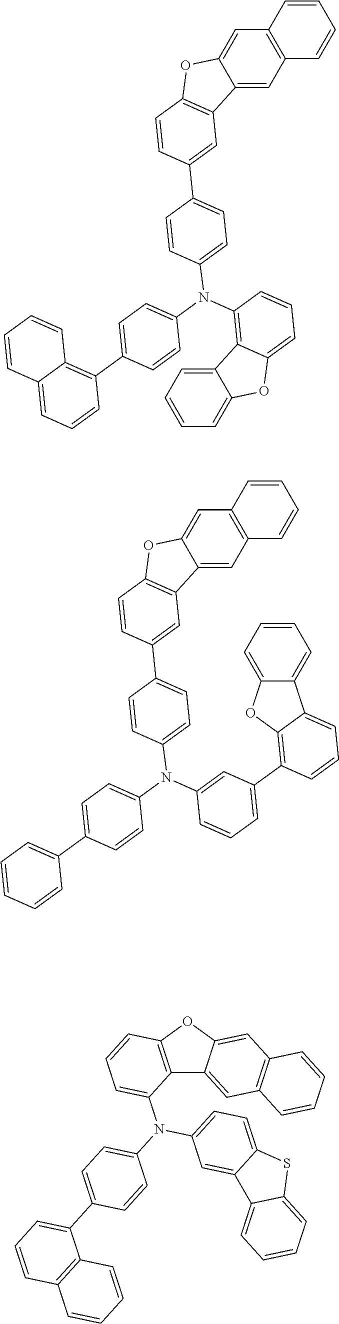 [SMILES: c1ccc2cc3c(cc2c1)oc1ccc(-c2ccc(N(c4ccc(-c5cccc6ccccc56)cc4)c4cccc5oc6ccccc6c45)cc2)cc13 ; c1ccc(-c2ccc(N(c3ccc(-c4ccc5oc6cc7ccccc7cc6c5c4)cc3)c3cccc(-c4cccc5c4oc4ccccc45)c3)cc2)cc1 ; c1ccc2cc3c(cc2c1)oc1cccc(N(c2ccc(-c4cccc5ccccc45)cc2)c2ccc4sc5ccccc5c4c2)c13] 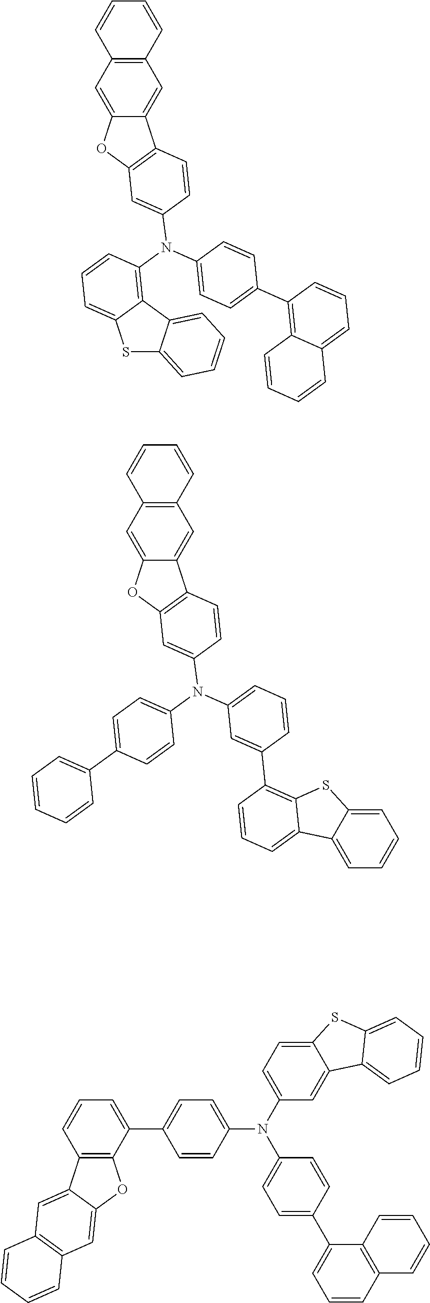 [SMILES: c1ccc2cc3c(cc2c1)oc1cc(N(c2ccc(-c4cccc5ccccc45)cc2)c2cccc4sc5ccccc5c24)ccc13 ; c1ccc(-c2ccc(N(c3cccc(-c4cccc5c4sc4ccccc45)c3)c3ccc4c(c3)oc3cc5ccccc5cc34)cc2)cc1 ; c1ccc2cc3c(cc2c1)oc1c(-c2ccc(N(c4ccc(-c5cccc6ccccc56)cc4)c4ccc5sc6ccccc6c5c4)cc2)cccc13] 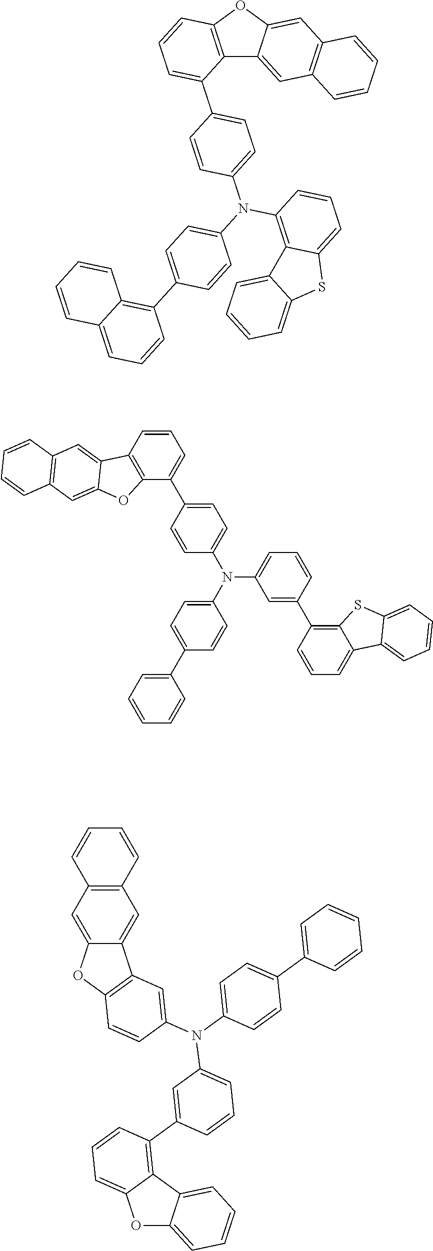 [SMILES: c1ccc2cc3c(cc2c1)oc1cccc(-c2ccc(N(c4ccc(-c5cccc6ccccc56)cc4)c4cccc5sc6ccccc6c45)cc2)c13 ; c1ccc(-c2ccc(N(c3ccc(-c4cccc5c4oc4cc6ccccc6cc45)cc3)c3cccc(-c4cccc5c4sc4ccccc45)c3)cc2)cc1 ; c1ccc(-c2ccc(N(c3cccc(-c4cccc5oc6ccccc6c45)c3)c3ccc4oc5cc6ccccc6cc5c4c3)cc2)cc1] 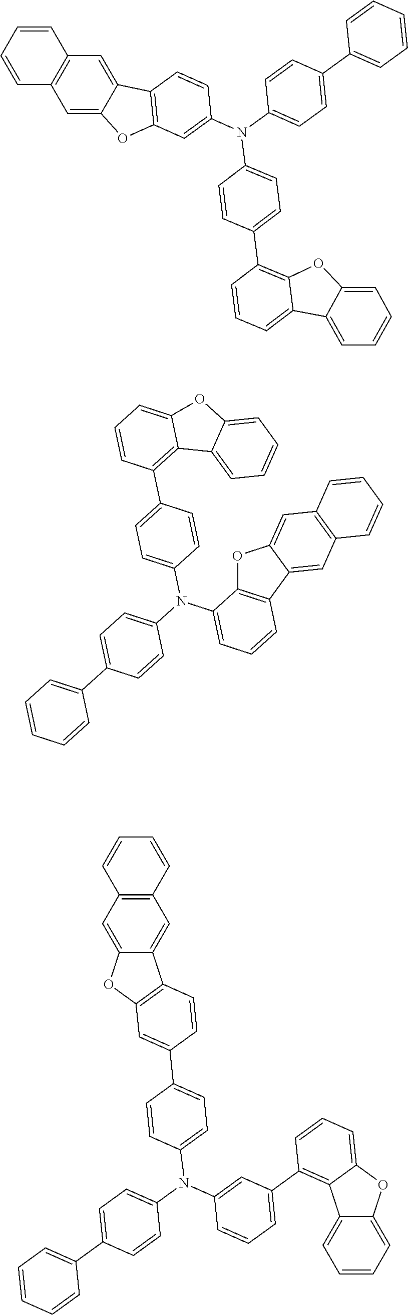 [SMILES: c1ccc(-c2ccc(N(c3ccc(-c4cccc5c4oc4ccccc45)cc3)c3ccc4c(c3)oc3cc5ccccc5cc34)cc2)cc1 ; c1ccc(-c2ccc(N(c3ccc(-c4cccc5oc6ccccc6c45)cc3)c3cccc4c3oc3cc5ccccc5cc34)cc2)cc1 ; c1ccc(-c2ccc(N(c3ccc(-c4ccc5c(c4)oc4cc6ccccc6cc45)cc3)c3cccc(-c4cccc5oc6ccccc6c45)c3)cc2)cc1] 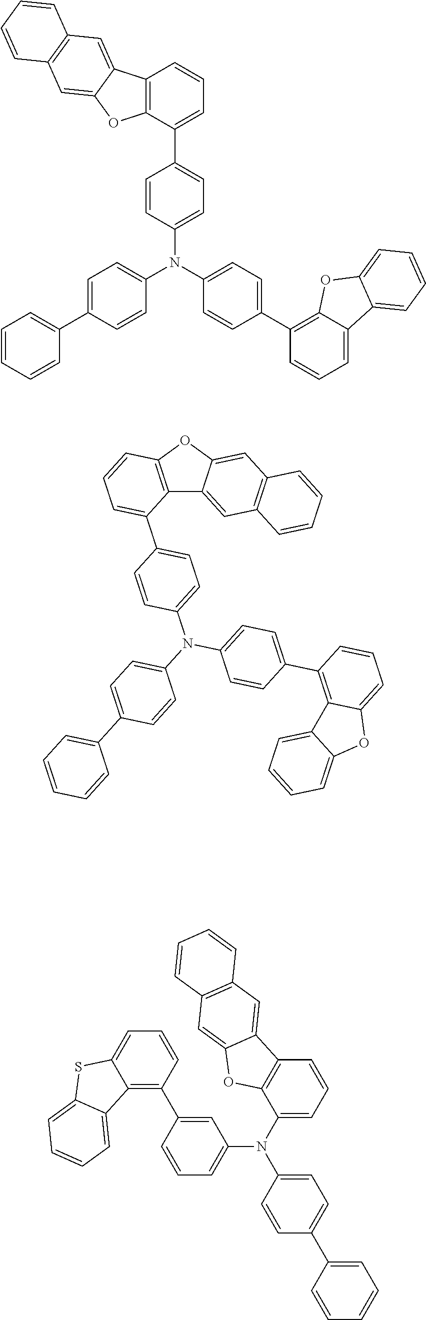 [SMILES: c1ccc(-c2ccc(N(c3ccc(-c4cccc5c4oc4ccccc45)cc3)c3ccc(-c4cccc5c4oc4cc6ccccc6cc45)cc3)cc2)cc1 ; c1ccc(-c2ccc(N(c3ccc(-c4cccc5oc6ccccc6c45)cc3)c3ccc(-c4cccc5oc6cc7ccccc7cc6c45)cc3)cc2)cc1 ; c1ccc(-c2ccc(N(c3cccc(-c4cccc5sc6ccccc6c45)c3)c3cccc4c3oc3cc5ccccc5cc34)cc2)cc1] 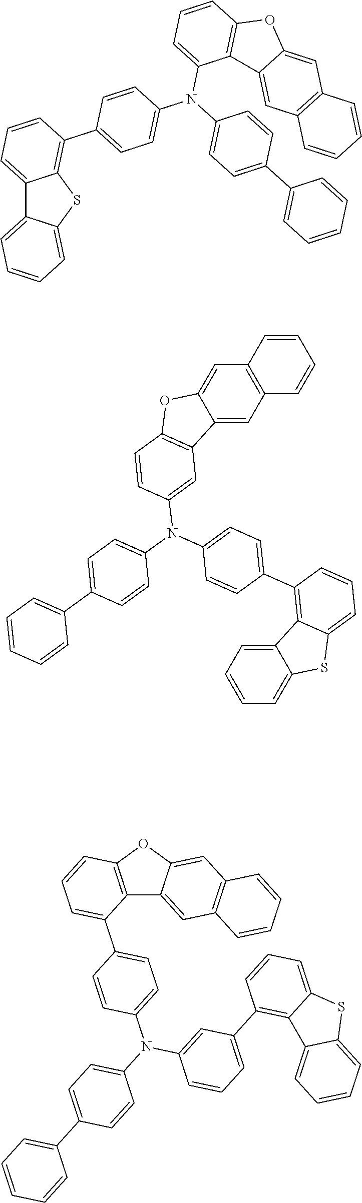 [SMILES: c1ccc(-c2ccc(N(c3ccc(-c4cccc5c4sc4ccccc45)cc3)c3cccc4oc5cc6ccccc6cc5c34)cc2)cc1 ; c1ccc(-c2ccc(N(c3ccc(-c4cccc5sc6ccccc6c45)cc3)c3ccc4oc5cc6ccccc6cc5c4c3)cc2)cc1 ; c1ccc(-c2ccc(N(c3ccc(-c4cccc5oc6cc7ccccc7cc6c45)cc3)c3cccc(-c4cccc5sc6ccccc6c45)c3)cc2)cc1] 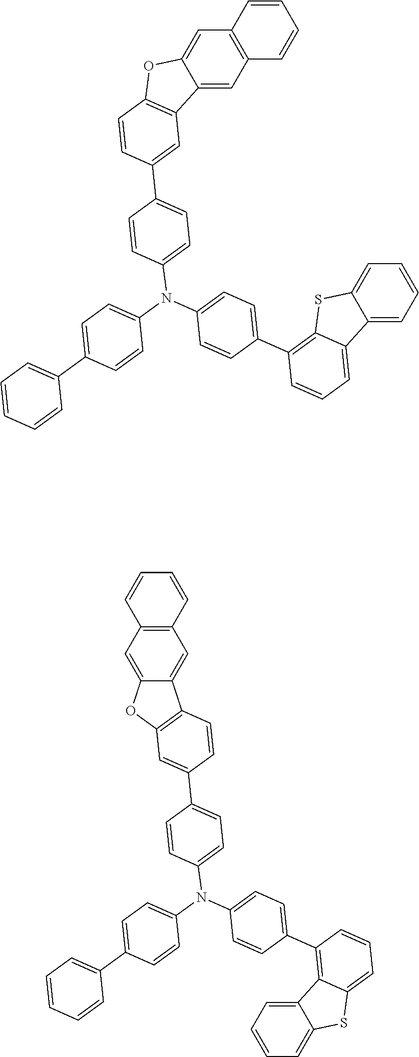 [SMILES: c1ccc(-c2ccc(N(c3ccc(-c4ccc5oc6cc7ccccc7cc6c5c4)cc3)c3ccc(-c4cccc5c4sc4ccccc45)cc3)cc2)cc1 ; c1ccc(-c2ccc(N(c3ccc(-c4ccc5c(c4)oc4cc6ccccc6cc45)cc3)c3ccc(-c4cccc5sc6ccccc6c45)cc3)cc2)cc1] 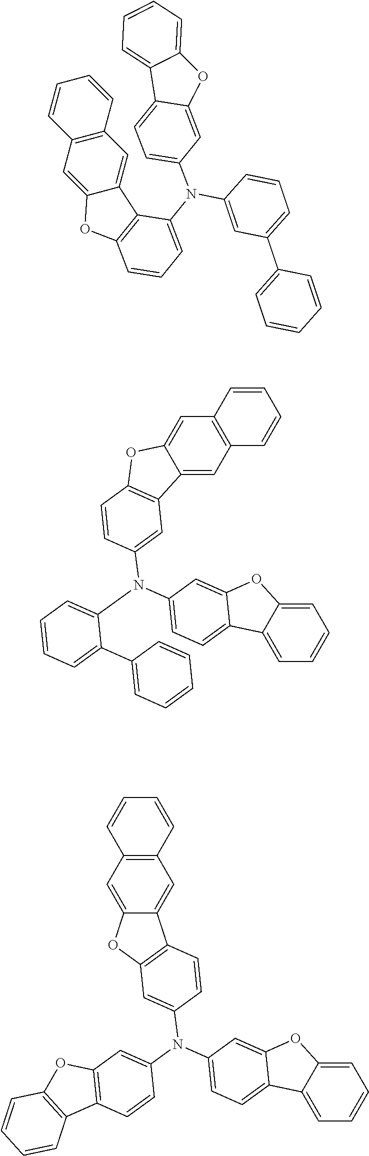 [SMILES: c1ccc(-c2cccc(N(c3ccc4c(c3)oc3ccccc34)c3cccc4oc5cc6ccccc6cc5c34)c2)cc1 ; c1ccc(-c2ccccc2N(c2ccc3c(c2)oc2ccccc23)c2ccc3oc4cc5ccccc5cc4c3c2)cc1 ; c1ccc2cc3c(cc2c1)oc1cc(N(c2ccc4c(c2)oc2ccccc24)c2ccc4c(c2)oc2ccccc24)ccc13] 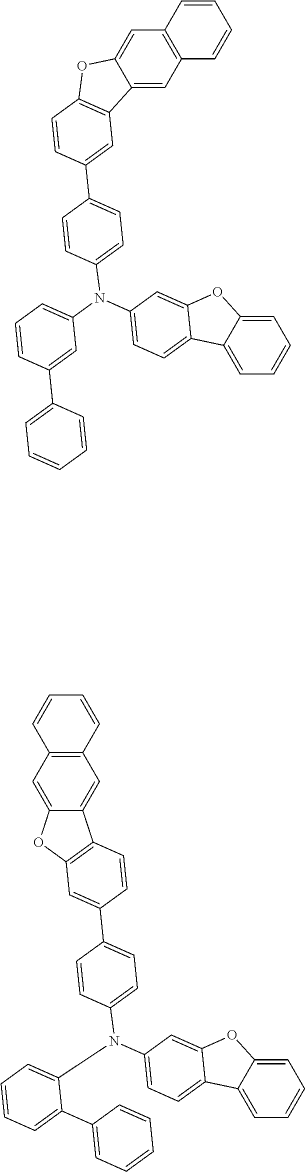 [SMILES: c1ccc(-c2cccc(N(c3ccc(-c4ccc5oc6cc7ccccc7cc6c5c4)cc3)c3ccc4c(c3)oc3ccccc34)c2)cc1 ; c1ccc(-c2ccccc2N(c2ccc(-c3ccc4c(c3)oc3cc5ccccc5cc34)cc2)c2ccc3c(c2)oc2ccccc23)cc1] 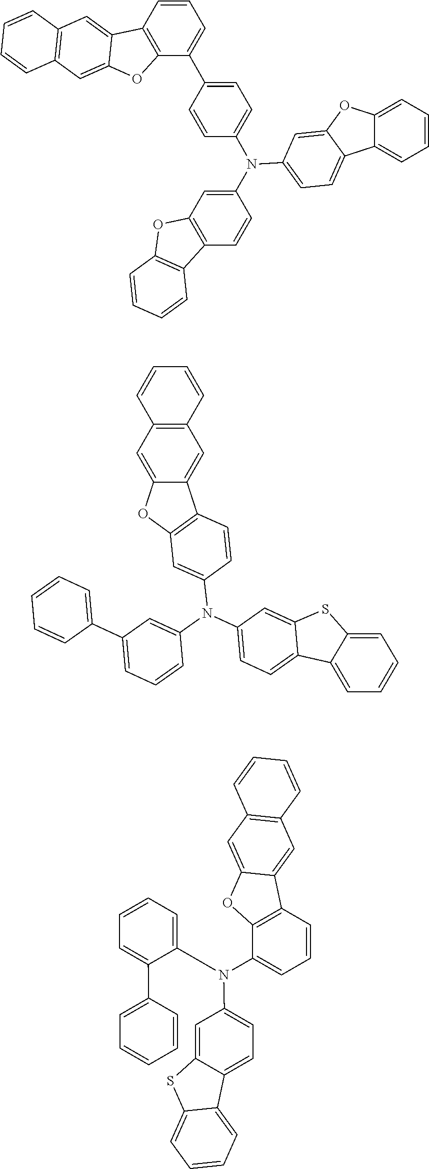 [SMILES: c1ccc2cc3c(cc2c1)oc1c(-c2ccc(N(c4ccc5c(c4)oc4ccccc45)c4ccc5c(c4)oc4ccccc45)cc2)cccc13 ; c1ccc(-c2cccc(N(c3ccc4c(c3)oc3cc5ccccc5cc34)c3ccc4c(c3)sc3ccccc34)c2)cc1 ; c1ccc(-c2ccccc2N(c2ccc3c(c2)sc2ccccc23)c2cccc3c2oc2cc4ccccc4cc23)cc1] 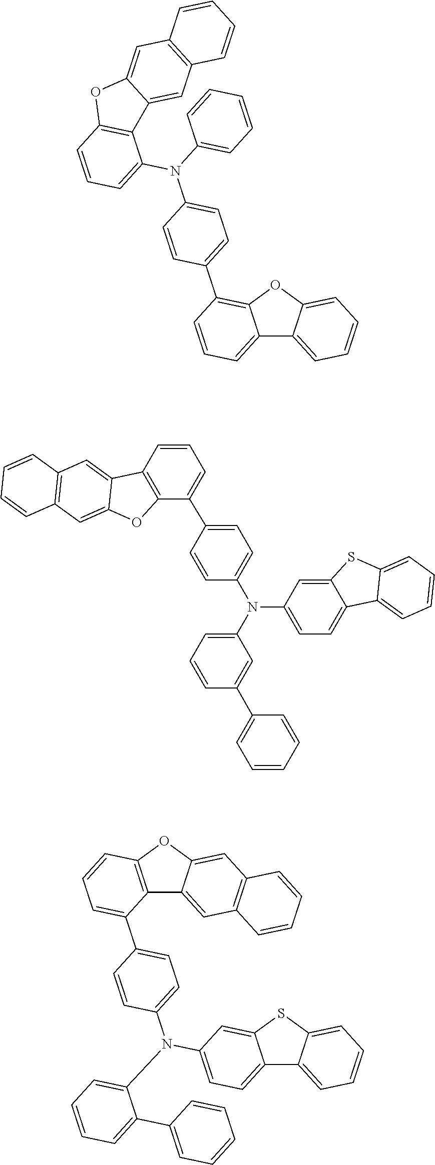 [SMILES: c1ccc(N(c2ccc(-c3cccc4c3oc3ccccc34)cc2)c2cccc3oc4cc5ccccc5cc4c23)cc1 ; c1ccc(-c2cccc(N(c3ccc(-c4cccc5c4oc4cc6ccccc6cc45)cc3)c3ccc4c(c3)sc3ccccc34)c2)cc1 ; c1ccc(-c2ccccc2N(c2ccc(-c3cccc4oc5cc6ccccc6cc5c34)cc2)c2ccc3c(c2)sc2ccccc23)cc1] 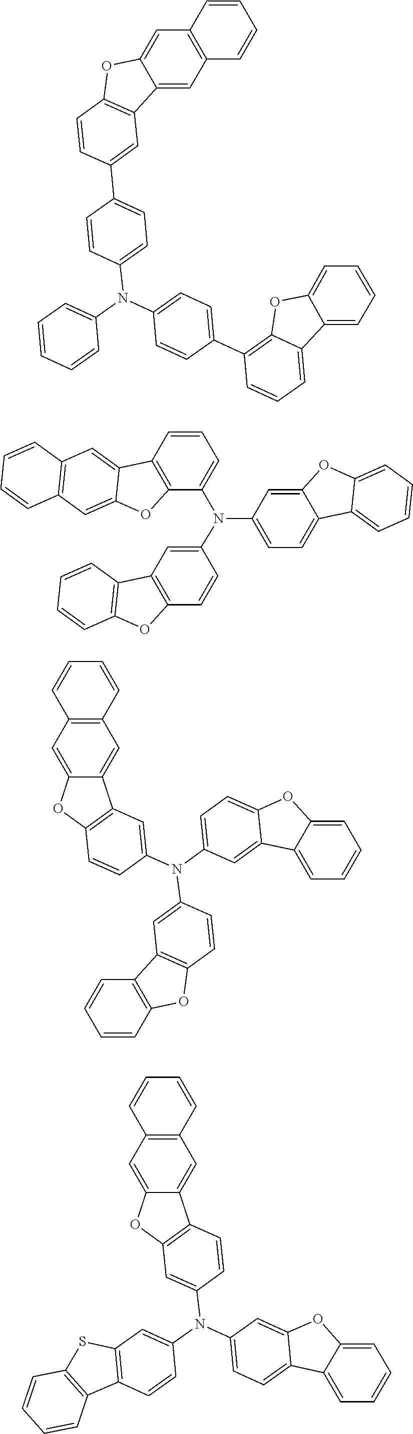 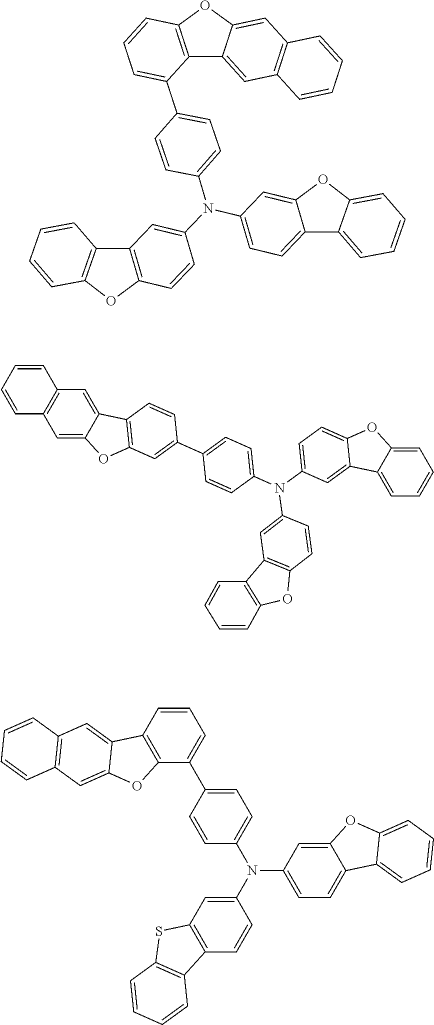 [SMILES: c1ccc2cc3c(cc2c1)oc1cccc(-c2ccc(N(c4ccc5c(c4)oc4ccccc45)c4ccc5oc6ccccc6c5c4)cc2)c13 ; c1ccc2cc3c(cc2c1)oc1cc(-c2ccc(N(c4ccc5oc6ccccc6c5c4)c4ccc5oc6ccccc6c5c4)cc2)ccc13 ; c1ccc2cc3c(cc2c1)oc1c(-c2ccc(N(c4ccc5c(c4)oc4ccccc45)c4ccc5c(c4)sc4ccccc45)cc2)cccc13] 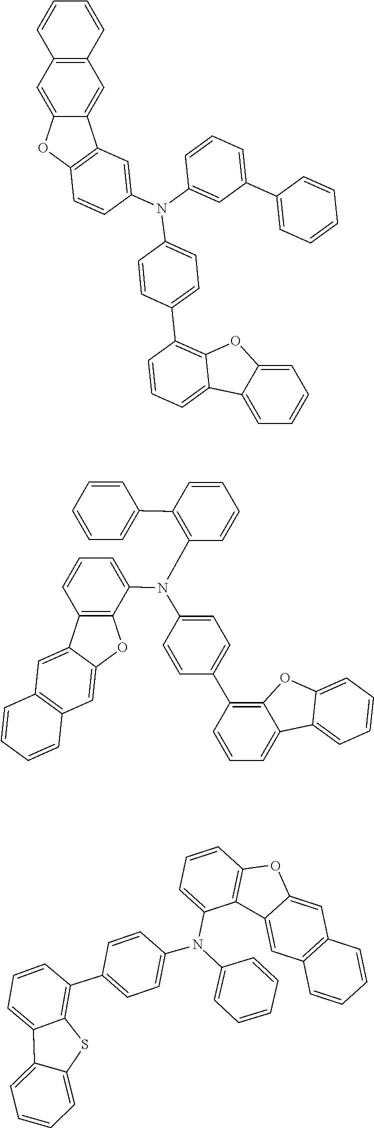 [SMILES: c1ccc(-c2cccc(N(c3ccc(-c4cccc5c4oc4ccccc45)cc3)c3ccc4oc5cc6ccccc6cc5c4c3)c2)cc1 ; c1ccc(-c2ccccc2N(c2ccc(-c3cccc4c3oc3ccccc34)cc2)c2cccc3c2oc2cc4ccccc4cc23)cc1 ; c1ccc(N(c2ccc(-c3cccc4c3sc3ccccc34)cc2)c2cccc3oc4cc5ccccc5cc4c23)cc1] 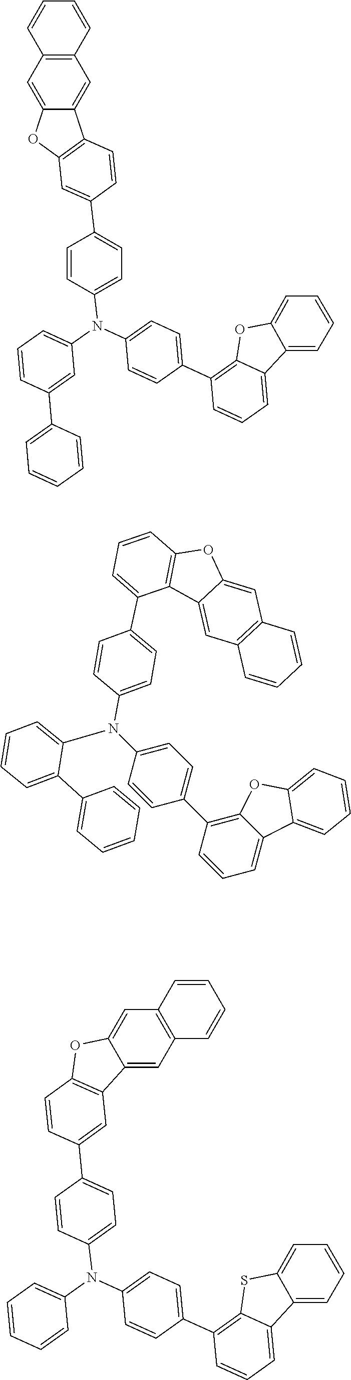 [SMILES: c1ccc(-c2cccc(N(c3ccc(-c4ccc5c(c4)oc4cc6ccccc6cc45)cc3)c3ccc(-c4cccc5c4oc4ccccc45)cc3)c2)cc1 ; c1ccc(-c2ccccc2N(c2ccc(-c3cccc4c3oc3ccccc34)cc2)c2ccc(-c3cccc4oc5cc6ccccc6cc5c34)cc2)cc1 ; c1ccc(N(c2ccc(-c3ccc4oc5cc6ccccc6cc5c4c3)cc2)c2ccc(-c3cccc4c3sc3ccccc34)cc2)cc1] 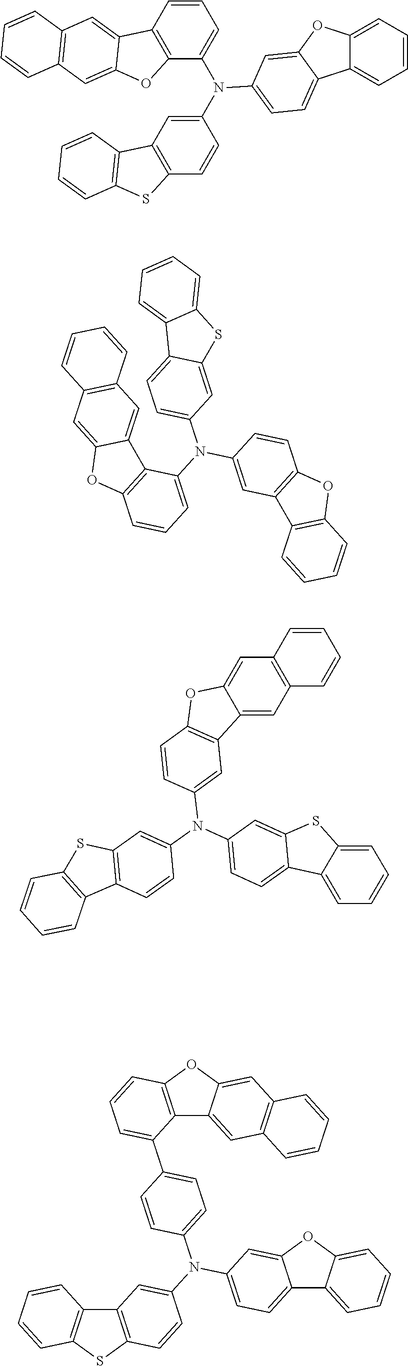 [SMILES: c1ccc2cc3c(cc2c1)oc1c(N(c2ccc4c(c2)oc2ccccc24)c2ccc4sc5ccccc5c4c2)cccc13 ; c1ccc2cc3c(cc2c1)oc1cccc(N(c2ccc4c(c2)sc2ccccc24)c2ccc4oc5ccccc5c4c2)c13 ; c1ccc2cc3c(cc2c1)oc1ccc(N(c2ccc4c(c2)sc2ccccc24)c2ccc4c(c2)sc2ccccc24)cc13 ; c1ccc2cc3c(cc2c1)oc1cccc(-c2ccc(N(c4ccc5c(c4)oc4ccccc45)c4ccc5sc6ccccc6c5c4)cc2)c13] 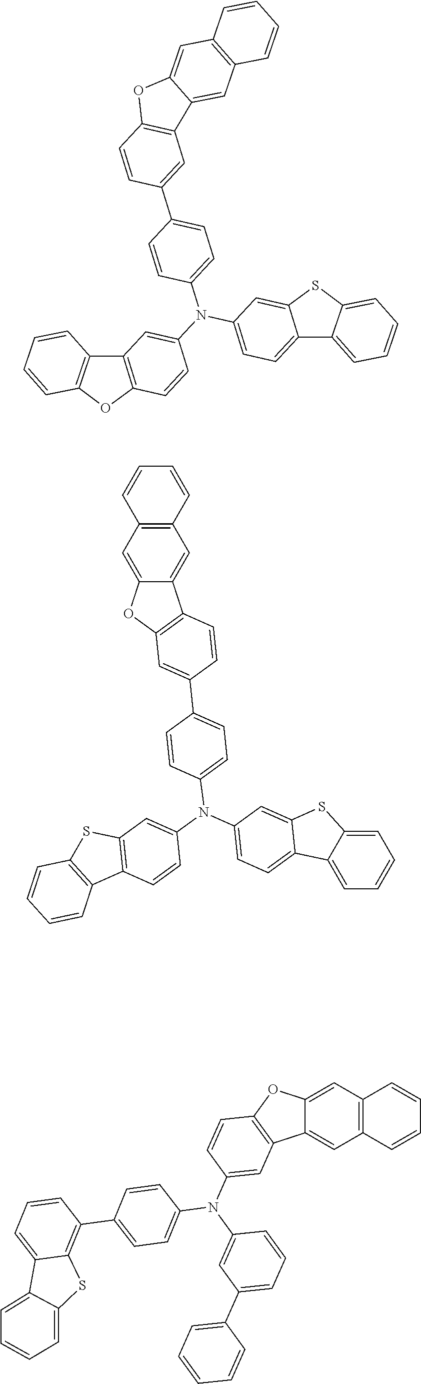 [SMILES: c1ccc2cc3c(cc2c1)oc1ccc(-c2ccc(N(c4ccc5c(c4)sc4ccccc45)c4ccc5oc6ccccc6c5c4)cc2)cc13 ; c1ccc2cc3c(cc2c1)oc1cc(-c2ccc(N(c4ccc5c(c4)sc4ccccc45)c4ccc5c(c4)sc4ccccc45)cc2)ccc13 ; c1ccc(-c2cccc(N(c3ccc(-c4cccc5c4sc4ccccc45)cc3)c3ccc4oc5cc6ccccc6cc5c4c3)c2)cc1] 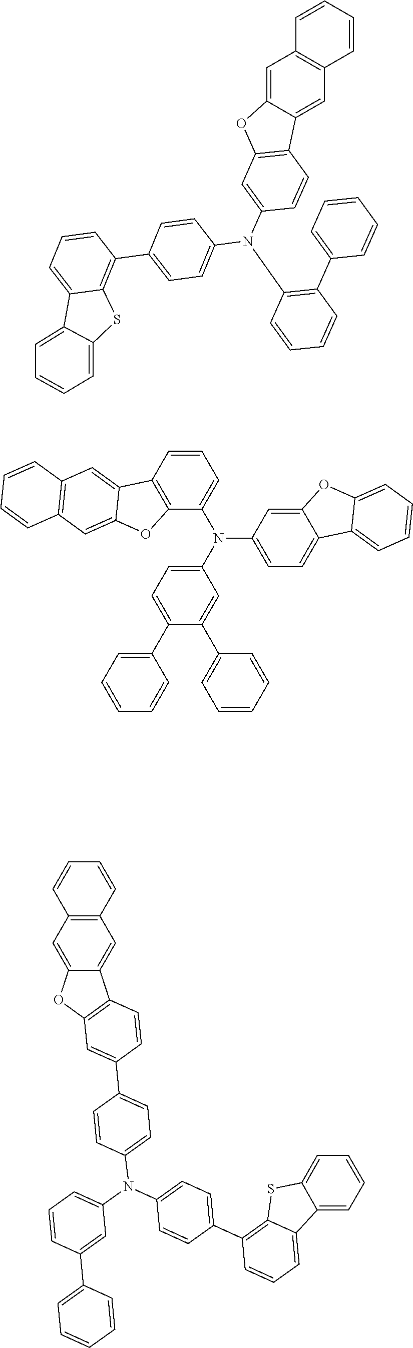 [SMILES: c1ccc(-c2ccccc2N(c2ccc(-c3cccc4c3sc3ccccc34)cc2)c2ccc3c(c2)oc2cc4ccccc4cc23)cc1 ; c1ccc(-c2ccc(N(c3ccc4c(c3)oc3ccccc34)c3cccc4c3oc3cc5ccccc5cc34)cc2-c2ccccc2)cc1 ; c1ccc(-c2cccc(N(c3ccc(-c4ccc5c(c4)oc4cc6ccccc6cc45)cc3)c3ccc(-c4cccc5c4sc4ccccc45)cc3)c2)cc1] 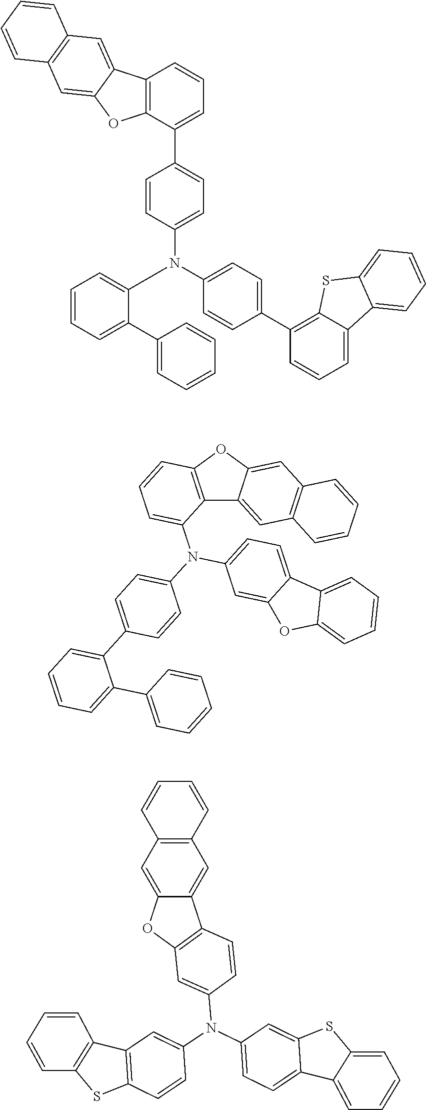 [SMILES: c1ccc(-c2ccccc2N(c2ccc(-c3cccc4c3oc3cc5ccccc5cc34)cc2)c2ccc(-c3cccc4c3sc3ccccc34)cc2)cc1 ; c1ccc(-c2ccccc2-c2ccc(N(c3ccc4c(c3)oc3ccccc34)c3cccc4oc5cc6ccccc6cc5c34)cc2)cc1 ; c1ccc2cc3c(cc2c1)oc1cc(N(c2ccc4c(c2)sc2ccccc24)c2ccc4sc5ccccc5c4c2)ccc13] 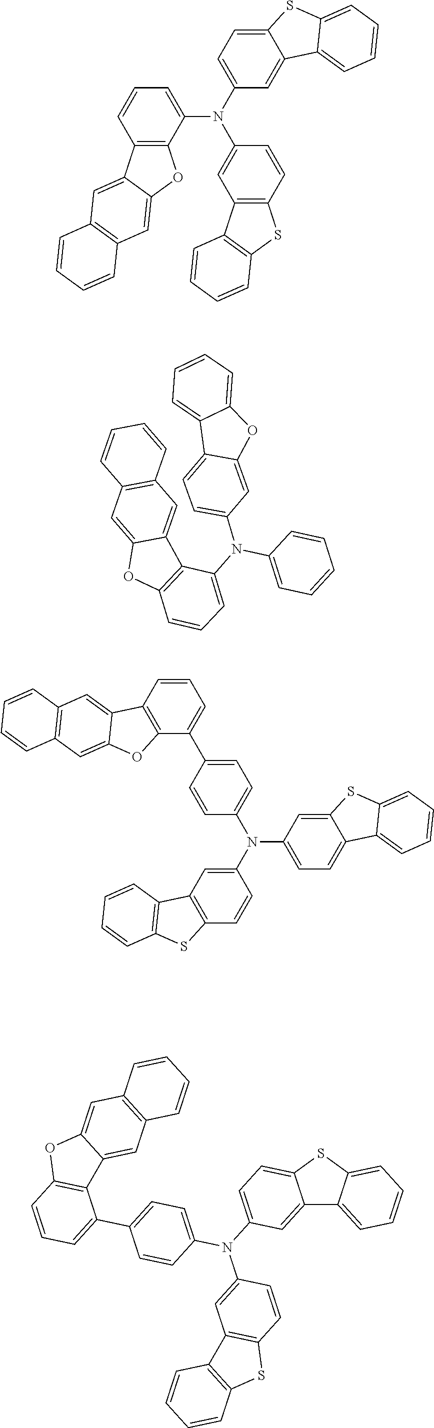 [SMILES: c1ccc2cc3c(cc2c1)oc1c(N(c2ccc4sc5ccccc5c4c2)c2ccc4sc5ccccc5c4c2)cccc13 ; c1ccc(N(c2ccc3c(c2)oc2ccccc23)c2cccc3oc4cc5ccccc5cc4c23)cc1 ; c1ccc2cc3c(cc2c1)oc1c(-c2ccc(N(c4ccc5c(c4)sc4ccccc45)c4ccc5sc6ccccc6c5c4)cc2)cccc13 ; c1ccc2cc3c(cc2c1)oc1cccc(-c2ccc(N(c4ccc5sc6ccccc6c5c4)c4ccc5sc6ccccc6c5c4)cc2)c13] 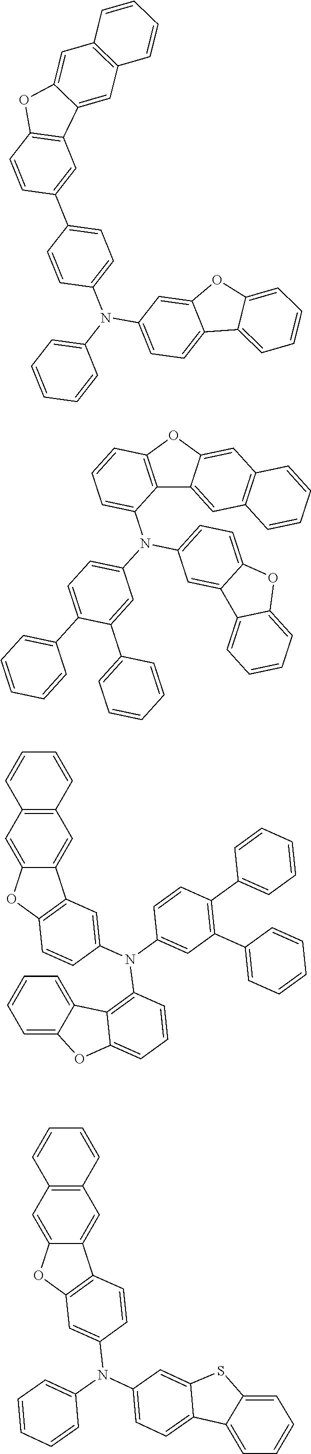 [SMILES: c1ccc(N(c2ccc(-c3ccc4oc5cc6ccccc6cc5c4c3)cc2)c2ccc3c(c2)oc2ccccc23)cc1 ; c1ccc(-c2ccc(N(c3ccc4oc5ccccc5c4c3)c3cccc4oc5cc6ccccc6cc5c34)cc2-c2ccccc2)cc1 ; c1ccc(-c2ccc(N(c3ccc4oc5cc6ccccc6cc5c4c3)c3cccc4oc5ccccc5c34)cc2-c2ccccc2)cc1 ; c1ccc(N(c2ccc3c(c2)oc2cc4ccccc4cc23)c2ccc3c(c2)sc2ccccc23)cc1] 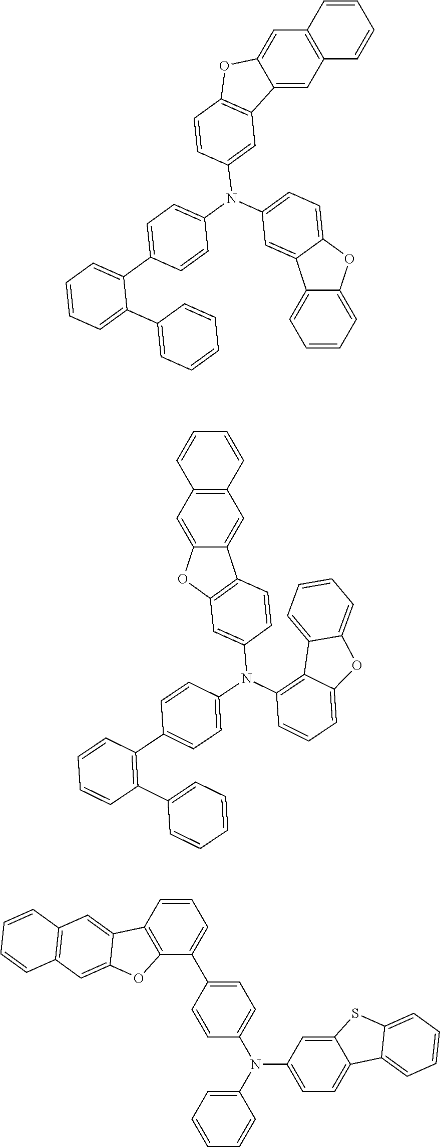 [SMILES: c1ccc(-c2ccccc2-c2ccc(N(c3ccc4oc5ccccc5c4c3)c3ccc4oc5cc6ccccc6cc5c4c3)cc2)cc1 ; c1ccc(-c2ccccc2-c2ccc(N(c3ccc4c(c3)oc3cc5ccccc5cc34)c3cccc4oc5ccccc5c34)cc2)cc1 ; c1ccc(N(c2ccc(-c3cccc4c3oc3cc5ccccc5cc34)cc2)c2ccc3c(c2)sc2ccccc23)cc1] 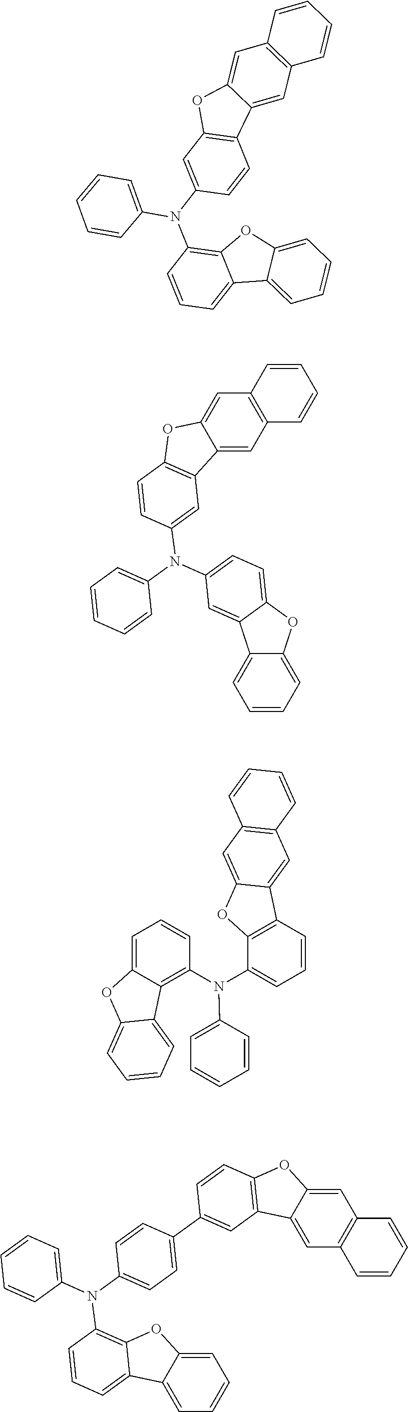 [SMILES: c1ccc(N(c2ccc3c(c2)oc2cc4ccccc4cc23)c2cccc3c2oc2ccccc23)cc1 ; c1ccc(N(c2ccc3oc4ccccc4c3c2)c2ccc3oc4cc5ccccc5cc4c3c2)cc1 ; c1ccc(N(c2cccc3c2oc2cc4ccccc4cc23)c2cccc3oc4ccccc4c23)cc1 ; c1ccc(N(c2ccc(-c3ccc4oc5cc6ccccc6cc5c4c3)cc2)c2cccc3c2oc2ccccc23)cc1] 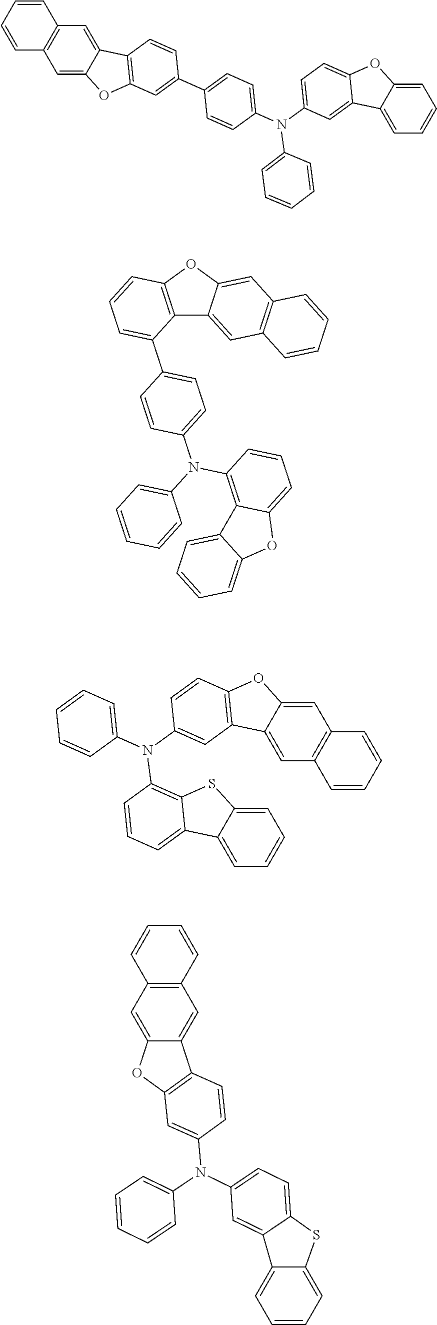 [SMILES: c1ccc(N(c2ccc(-c3ccc4c(c3)oc3cc5ccccc5cc34)cc2)c2ccc3oc4ccccc4c3c2)cc1 ; c1ccc(N(c2ccc(-c3cccc4oc5cc6ccccc6cc5c34)cc2)c2cccc3oc4ccccc4c23)cc1 ; c1ccc(N(c2ccc3oc4cc5ccccc5cc4c3c2)c2cccc3c2sc2ccccc23)cc1 ; c1ccc(N(c2ccc3c(c2)oc2cc4ccccc4cc23)c2ccc3sc4ccccc4c3c2)cc1] 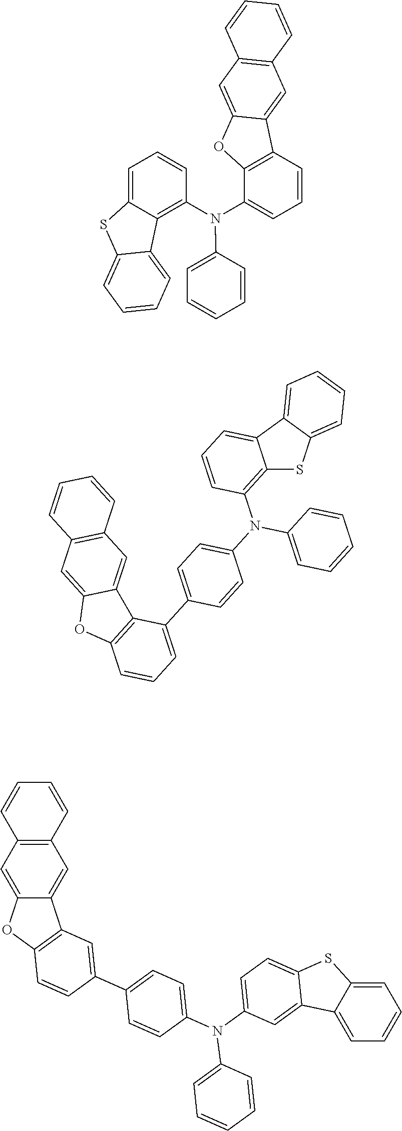 [SMILES: c1ccc(N(c2cccc3c2oc2cc4ccccc4cc23)c2cccc3sc4ccccc4c23)cc1 ; c1ccc(N(c2ccc(-c3cccc4oc5cc6ccccc6cc5c34)cc2)c2cccc3c2sc2ccccc23)cc1 ; c1ccc(N(c2ccc(-c3ccc4oc5cc6ccccc6cc5c4c3)cc2)c2ccc3sc4ccccc4c3c2)cc1] 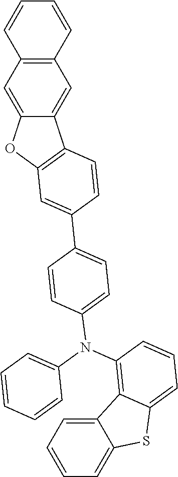 [SMILES: c1ccc(N(c2ccc(-c3ccc4c(c3)oc3cc5ccccc5cc34)cc2)c2cccc3sc4ccccc4c23)cc1] 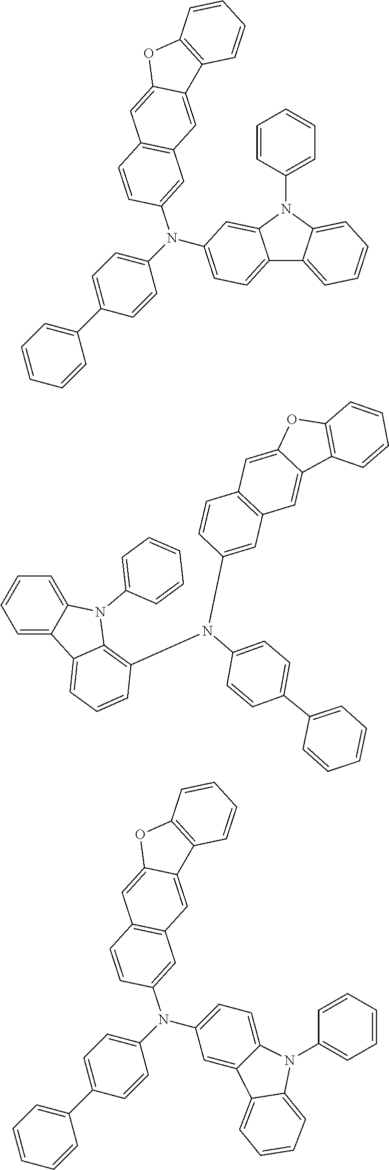 [SMILES: c1ccc(-c2ccc(N(c3ccc4cc5oc6ccccc6c5cc4c3)c3ccc4c5ccccc5n(-c5ccccc5)c4c3)cc2)cc1 ; c1ccc(-c2ccc(N(c3ccc4cc5oc6ccccc6c5cc4c3)c3cccc4c5ccccc5n(-c5ccccc5)c34)cc2)cc1 ; c1ccc(-c2ccc(N(c3ccc4cc5oc6ccccc6c5cc4c3)c3ccc4c(c3)c3ccccc3n4-c3ccccc3)cc2)cc1] 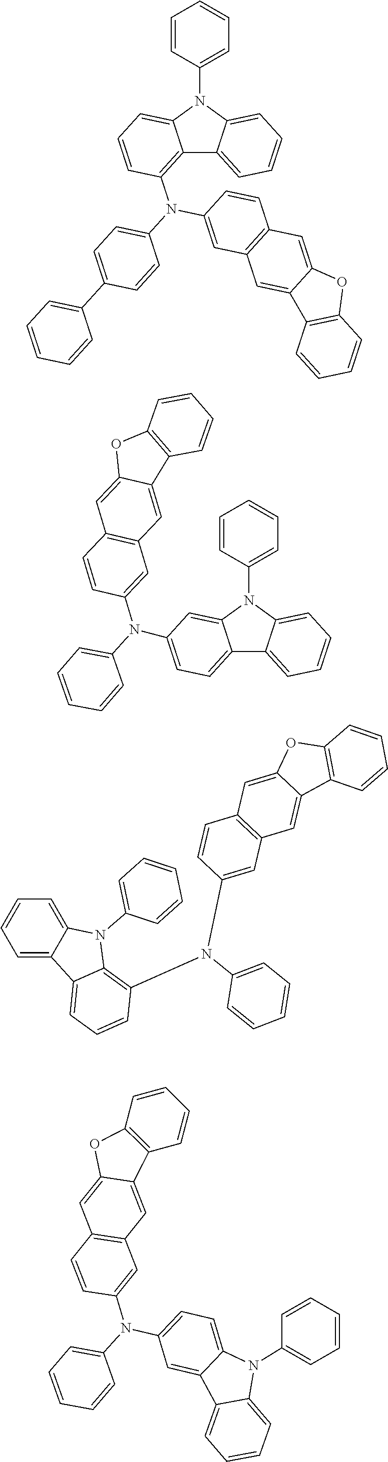 [SMILES: c1ccc(-c2ccc(N(c3ccc4cc5oc6ccccc6c5cc4c3)c3cccc4c3c3ccccc3n4-c3ccccc3)cc2)cc1 ; c1ccc(N(c2ccc3cc4oc5ccccc5c4cc3c2)c2ccc3c4ccccc4n(-c4ccccc4)c3c2)cc1 ; c1ccc(N(c2ccc3cc4oc5ccccc5c4cc3c2)c2cccc3c4ccccc4n(-c4ccccc4)c23)cc1 ; c1ccc(N(c2ccc3cc4oc5ccccc5c4cc3c2)c2ccc3c(c2)c2ccccc2n3-c2ccccc2)cc1] 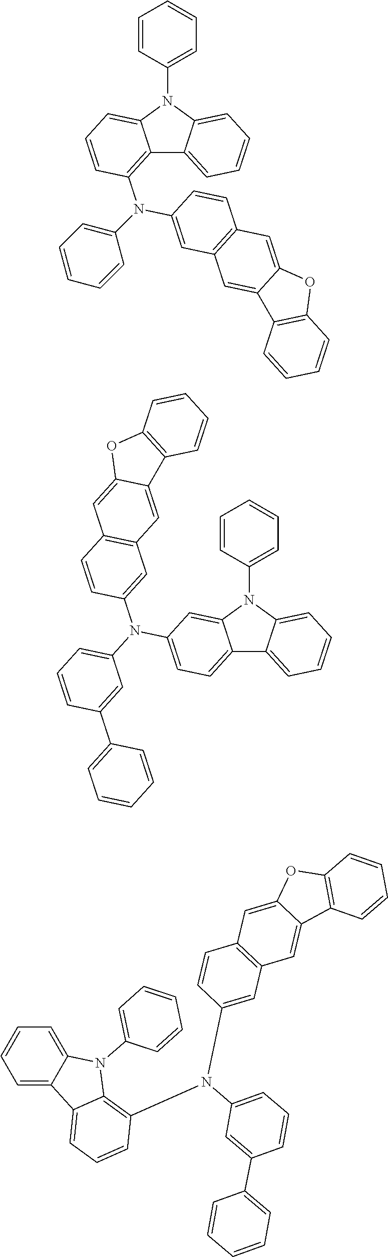 [SMILES: c1ccc(N(c2ccc3cc4oc5ccccc5c4cc3c2)c2cccc3c2c2ccccc2n3-c2ccccc2)cc1 ; c1ccc(-c2cccc(N(c3ccc4cc5oc6ccccc6c5cc4c3)c3ccc4c5ccccc5n(-c5ccccc5)c4c3)c2)cc1 ; c1ccc(-c2cccc(N(c3ccc4cc5oc6ccccc6c5cc4c3)c3cccc4c5ccccc5n(-c5ccccc5)c34)c2)cc1] 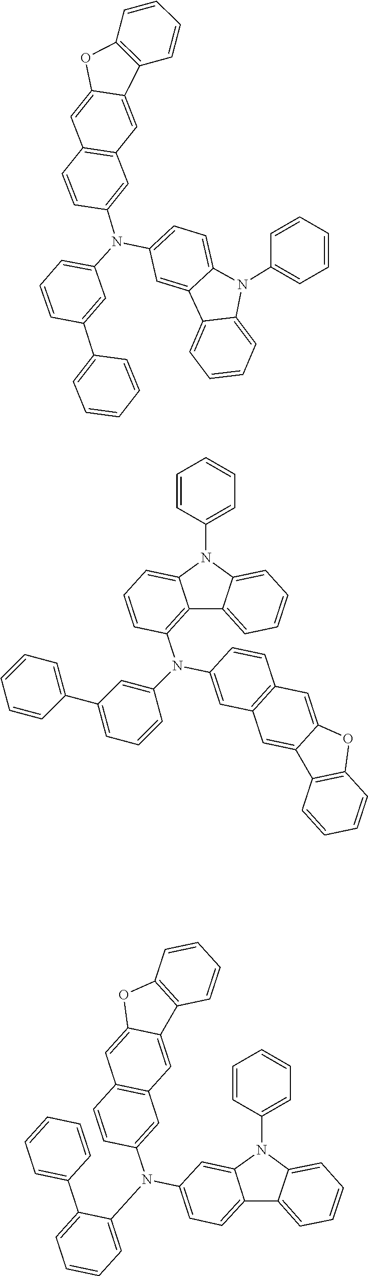 [SMILES: c1ccc(-c2cccc(N(c3ccc4cc5oc6ccccc6c5cc4c3)c3ccc4c(c3)c3ccccc3n4-c3ccccc3)c2)cc1 ; c1ccc(-c2cccc(N(c3ccc4cc5oc6ccccc6c5cc4c3)c3cccc4c3c3ccccc3n4-c3ccccc3)c2)cc1 ; c1ccc(-c2ccccc2N(c2ccc3cc4oc5ccccc5c4cc3c2)c2ccc3c4ccccc4n(-c4ccccc4)c3c2)cc1] 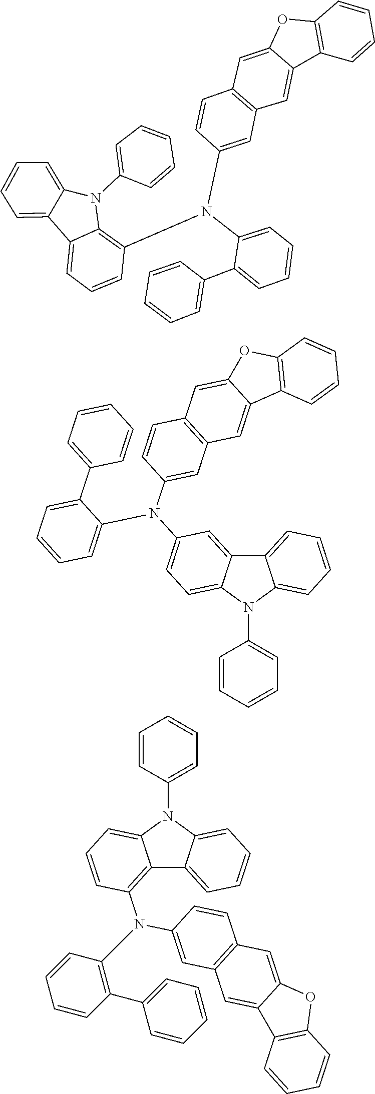 [SMILES: c1ccc(-c2ccccc2N(c2ccc3cc4oc5ccccc5c4cc3c2)c2cccc3c4ccccc4n(-c4ccccc4)c23)cc1 ; c1ccc(-c2ccccc2N(c2ccc3cc4oc5ccccc5c4cc3c2)c2ccc3c(c2)c2ccccc2n3-c2ccccc2)cc1 ; c1ccc(-c2ccccc2N(c2ccc3cc4oc5ccccc5c4cc3c2)c2cccc3c2c2ccccc2n3-c2ccccc2)cc1] 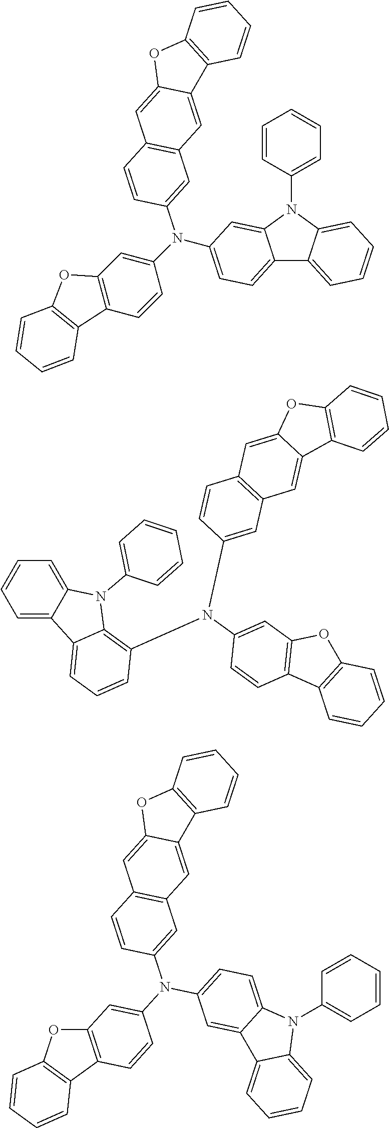 [SMILES: c1ccc(-n2c3ccccc3c3ccc(N(c4ccc5cc6oc7ccccc7c6cc5c4)c4ccc5c(c4)oc4ccccc45)cc32)cc1 ; c1ccc(-n2c3ccccc3c3cccc(N(c4ccc5cc6oc7ccccc7c6cc5c4)c4ccc5c(c4)oc4ccccc45)c32)cc1 ; c1ccc(-n2c3ccccc3c3cc(N(c4ccc5cc6oc7ccccc7c6cc5c4)c4ccc5c(c4)oc4ccccc45)ccc32)cc1] 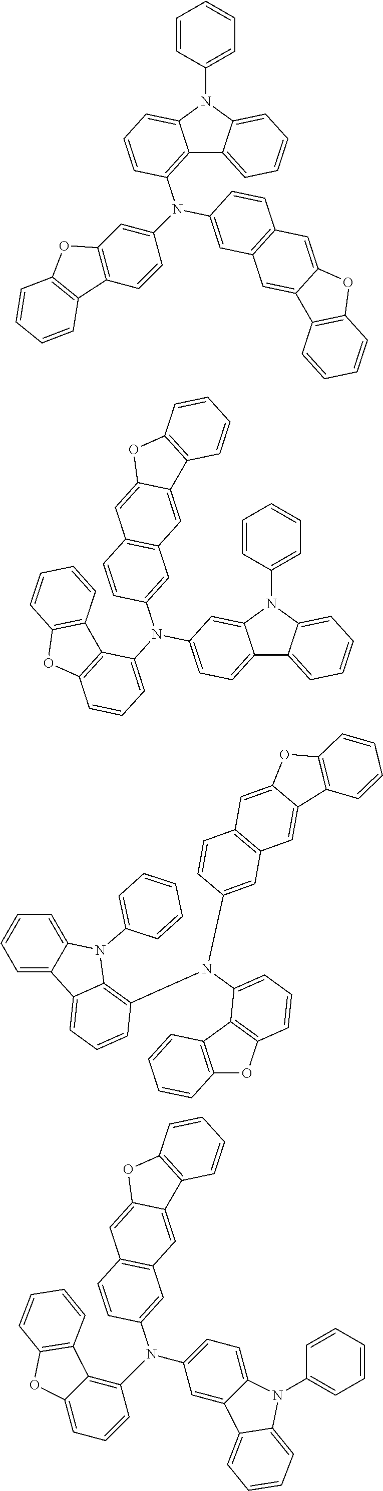 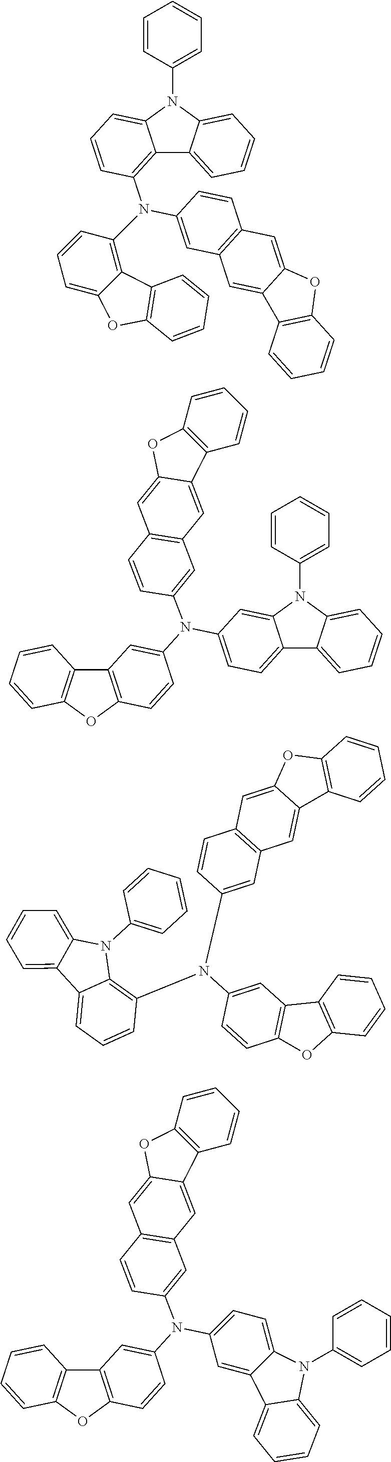 [SMILES: c1ccc(-n2c3ccccc3c3c(N(c4ccc5cc6oc7ccccc7c6cc5c4)c4cccc5oc6ccccc6c45)cccc32)cc1 ; c1ccc(-n2c3ccccc3c3ccc(N(c4ccc5cc6oc7ccccc7c6cc5c4)c4ccc5oc6ccccc6c5c4)cc32)cc1 ; c1ccc(-n2c3ccccc3c3cccc(N(c4ccc5cc6oc7ccccc7c6cc5c4)c4ccc5oc6ccccc6c5c4)c32)cc1 ; c1ccc(-n2c3ccccc3c3cc(N(c4ccc5cc6oc7ccccc7c6cc5c4)c4ccc5oc6ccccc6c5c4)ccc32)cc1] 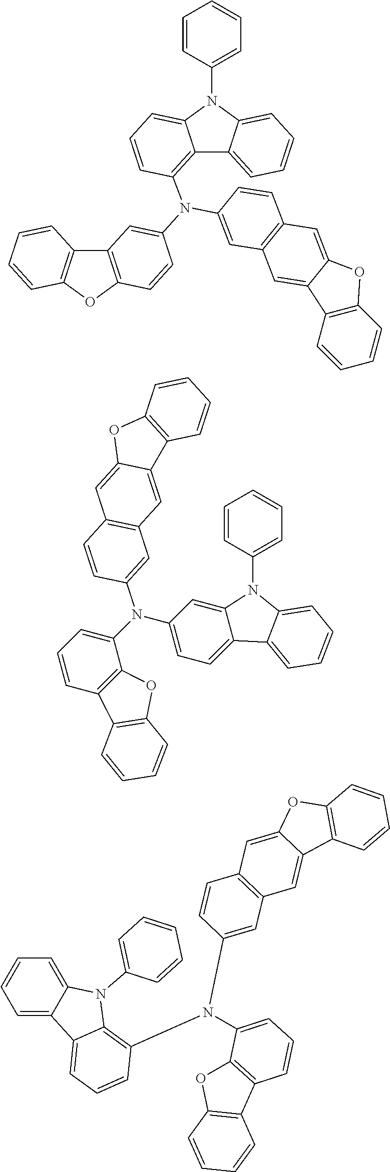 [SMILES: c1ccc(-n2c3ccccc3c3c(N(c4ccc5cc6oc7ccccc7c6cc5c4)c4ccc5oc6ccccc6c5c4)cccc32)cc1 ; c1ccc(-n2c3ccccc3c3ccc(N(c4ccc5cc6oc7ccccc7c6cc5c4)c4cccc5c4oc4ccccc45)cc32)cc1 ; c1ccc(-n2c3ccccc3c3cccc(N(c4ccc5cc6oc7ccccc7c6cc5c4)c4cccc5c4oc4ccccc45)c32)cc1] 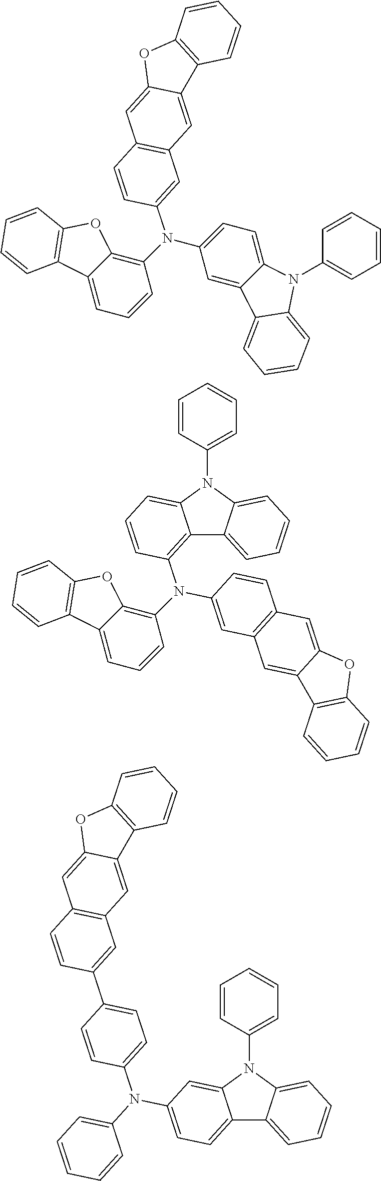 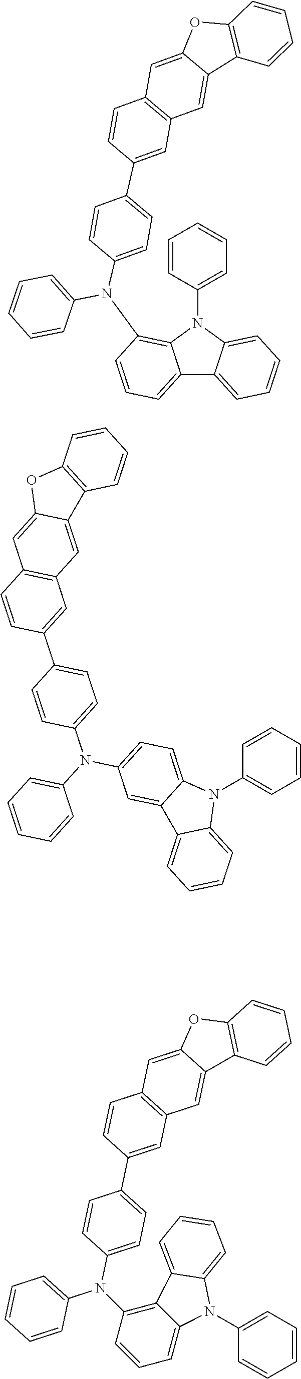 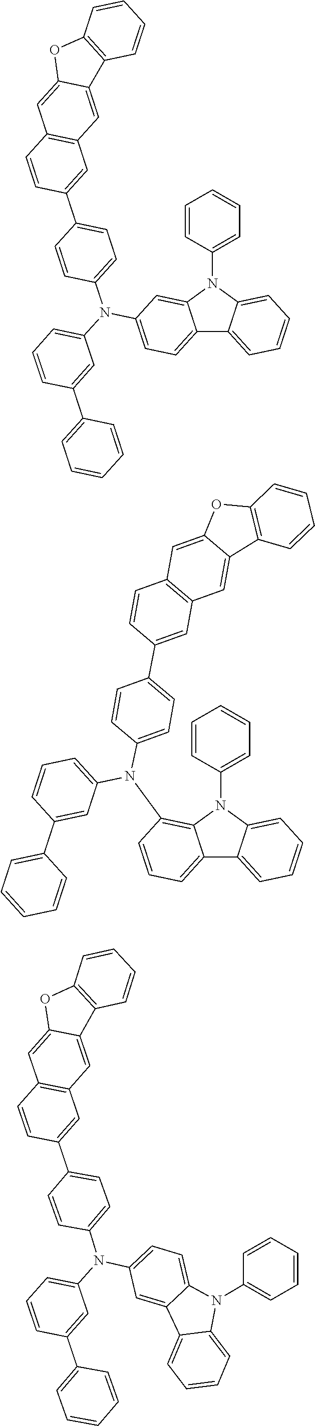 [SMILES: c1ccc(-c2cccc(N(c3ccc(-c4ccc5cc6oc7ccccc7c6cc5c4)cc3)c3ccc4c5ccccc5n(-c5ccccc5)c4c3)c2)cc1 ; c1ccc(-c2cccc(N(c3ccc(-c4ccc5cc6oc7ccccc7c6cc5c4)cc3)c3cccc4c5ccccc5n(-c5ccccc5)c34)c2)cc1 ; c1ccc(-c2cccc(N(c3ccc(-c4ccc5cc6oc7ccccc7c6cc5c4)cc3)c3ccc4c(c3)c3ccccc3n4-c3ccccc3)c2)cc1] 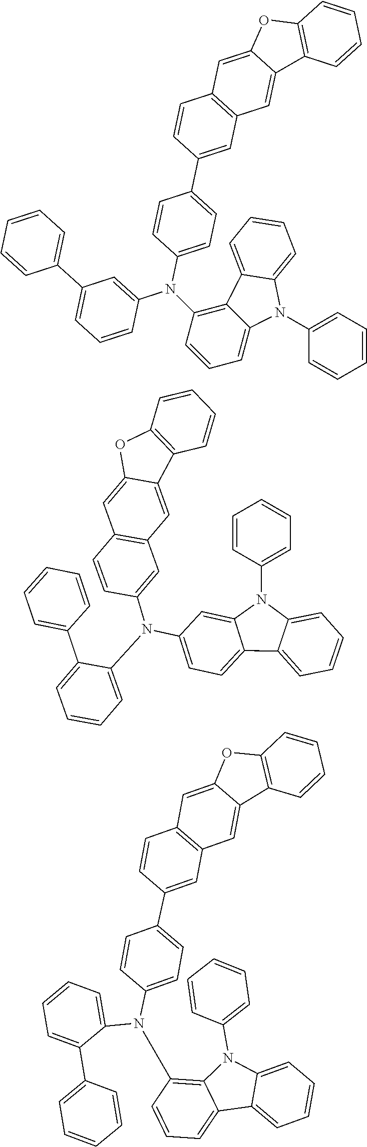 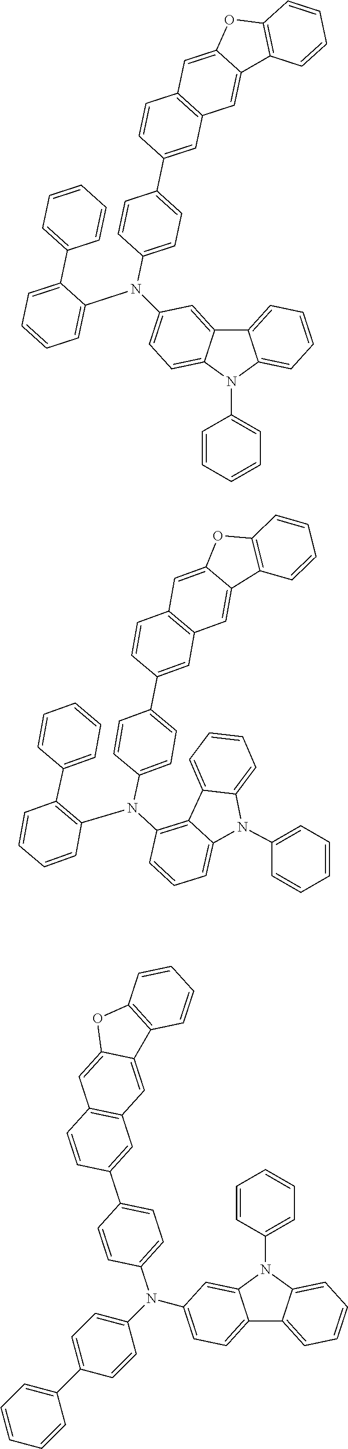 [SMILES: c1ccc(-c2ccccc2N(c2ccc(-c3ccc4cc5oc6ccccc6c5cc4c3)cc2)c2ccc3c(c2)c2ccccc2n3-c2ccccc2)cc1 ; c1ccc(-c2ccccc2N(c2ccc(-c3ccc4cc5oc6ccccc6c5cc4c3)cc2)c2cccc3c2c2ccccc2n3-c2ccccc2)cc1 ; c1ccc(-c2ccc(N(c3ccc(-c4ccc5cc6oc7ccccc7c6cc5c4)cc3)c3ccc4c5ccccc5n(-c5ccccc5)c4c3)cc2)cc1] 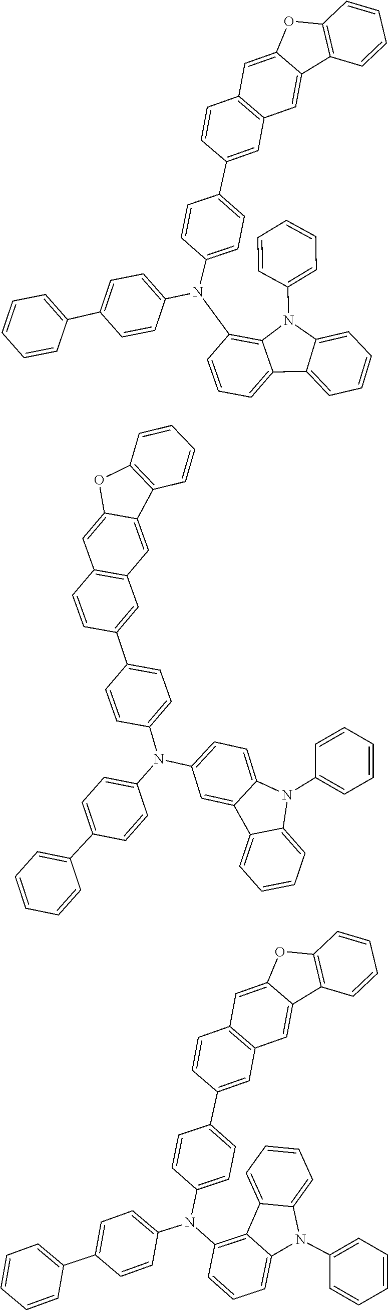 [SMILES: c1ccc(-c2ccc(N(c3ccc(-c4ccc5cc6oc7ccccc7c6cc5c4)cc3)c3cccc4c5ccccc5n(-c5ccccc5)c34)cc2)cc1 ; c1ccc(-c2ccc(N(c3ccc(-c4ccc5cc6oc7ccccc7c6cc5c4)cc3)c3ccc4c(c3)c3ccccc3n4-c3ccccc3)cc2)cc1 ; c1ccc(-c2ccc(N(c3ccc(-c4ccc5cc6oc7ccccc7c6cc5c4)cc3)c3cccc4c3c3ccccc3n4-c3ccccc3)cc2)cc1] 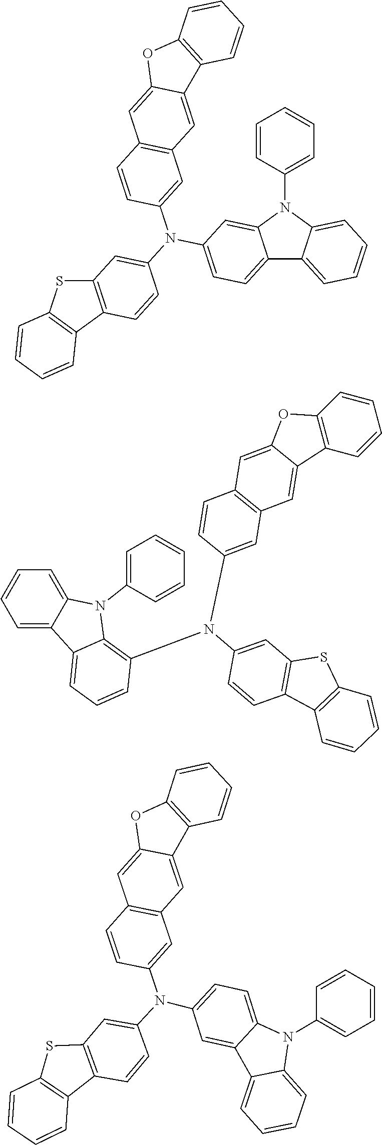 [SMILES: c1ccc(-n2c3ccccc3c3ccc(N(c4ccc5cc6oc7ccccc7c6cc5c4)c4ccc5c(c4)sc4ccccc45)cc32)cc1 ; c1ccc(-n2c3ccccc3c3cccc(N(c4ccc5cc6oc7ccccc7c6cc5c4)c4ccc5c(c4)sc4ccccc45)c32)cc1 ; c1ccc(-n2c3ccccc3c3cc(N(c4ccc5cc6oc7ccccc7c6cc5c4)c4ccc5c(c4)sc4ccccc45)ccc32)cc1] 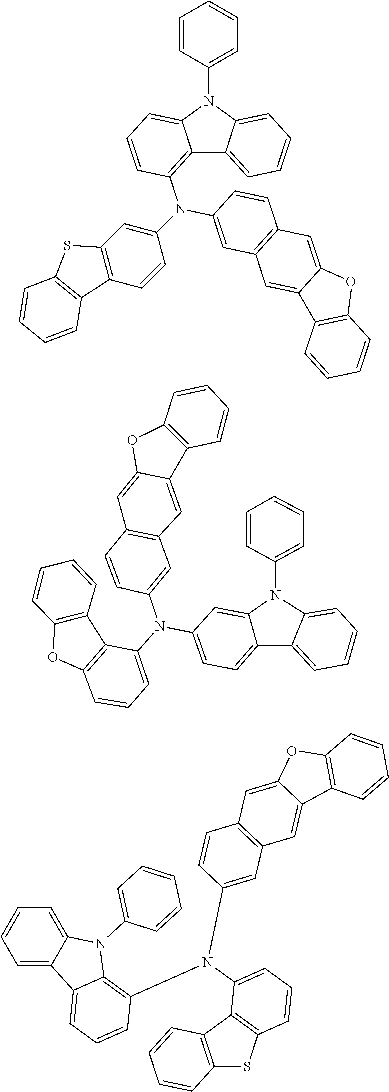 [SMILES: c1ccc(-n2c3ccccc3c3c(N(c4ccc5cc6oc7ccccc7c6cc5c4)c4ccc5c(c4)sc4ccccc45)cccc32)cc1 ; c1ccc(-n2c3ccccc3c3ccc(N(c4ccc5cc6oc7ccccc7c6cc5c4)c4cccc5oc6ccccc6c45)cc32)cc1 ; c1ccc(-n2c3ccccc3c3cccc(N(c4ccc5cc6oc7ccccc7c6cc5c4)c4cccc5sc6ccccc6c45)c32)cc1] 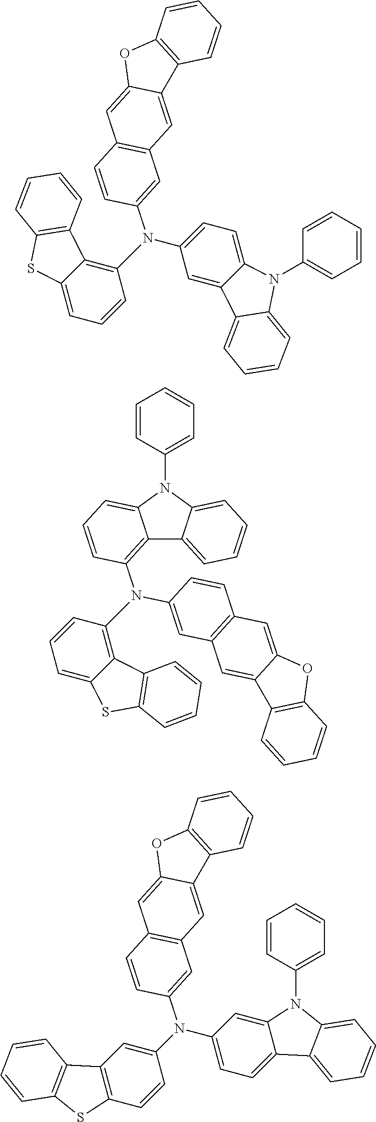 [SMILES: c1ccc(-n2c3ccccc3c3cc(N(c4ccc5cc6oc7ccccc7c6cc5c4)c4cccc5sc6ccccc6c45)ccc32)cc1 ; c1ccc(-n2c3ccccc3c3c(N(c4ccc5cc6oc7ccccc7c6cc5c4)c4cccc5sc6ccccc6c45)cccc32)cc1 ; c1ccc(-n2c3ccccc3c3ccc(N(c4ccc5cc6oc7ccccc7c6cc5c4)c4ccc5sc6ccccc6c5c4)cc32)cc1] 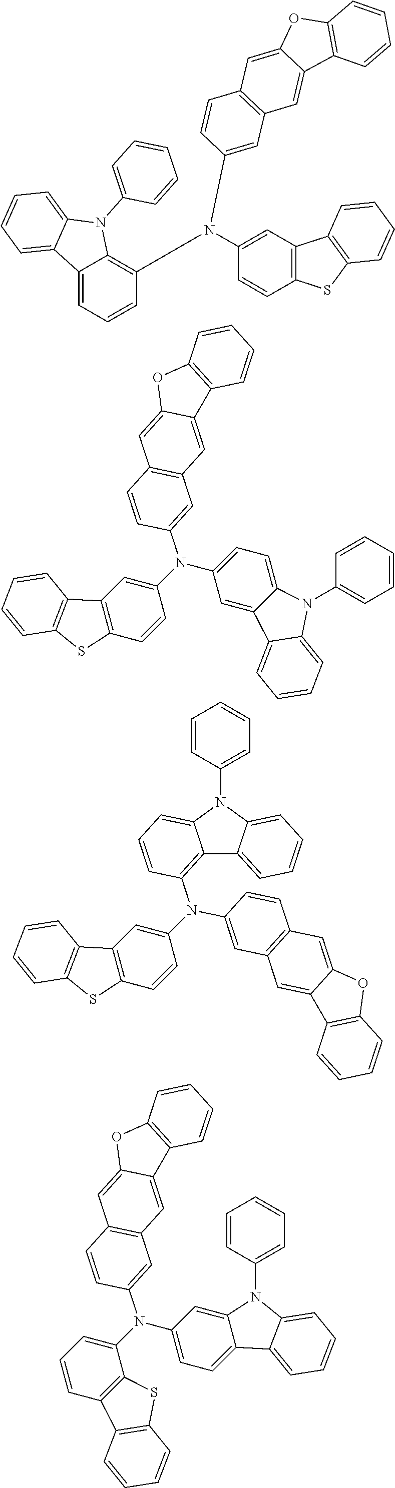 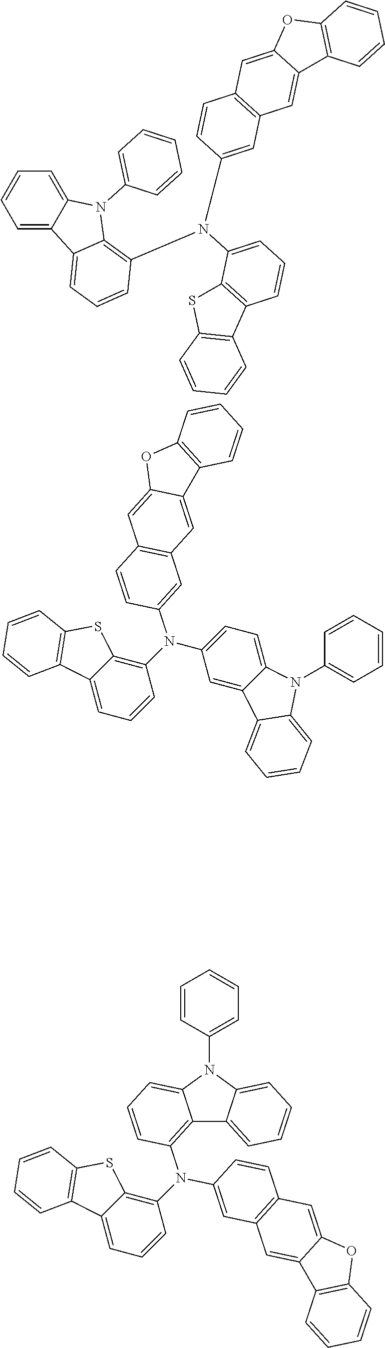 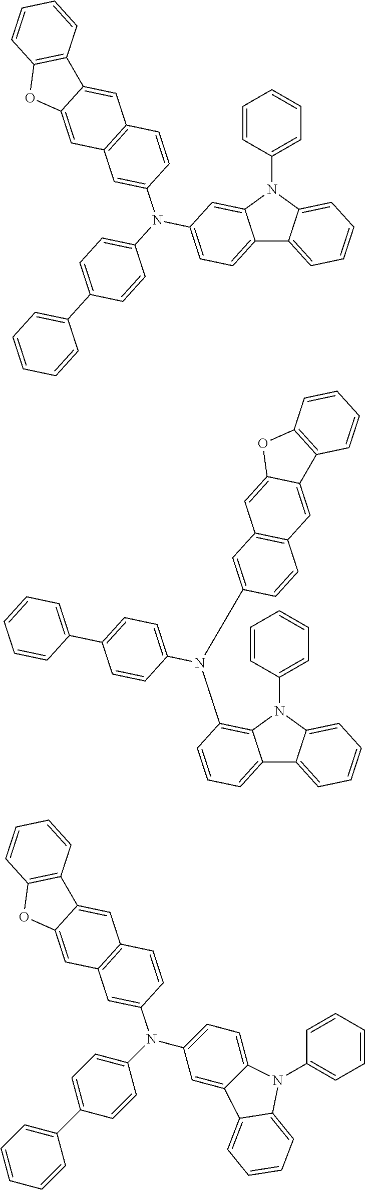 [SMILES: c1ccc(-c2ccc(N(c3ccc4cc5c(cc4c3)oc3ccccc35)c3ccc4c5ccccc5n(-c5ccccc5)c4c3)cc2)cc1 ; c1ccc(-c2ccc(N(c3ccc4cc5c(cc4c3)oc3ccccc35)c3cccc4c5ccccc5n(-c5ccccc5)c34)cc2)cc1 ; c1ccc(-c2ccc(N(c3ccc4cc5c(cc4c3)oc3ccccc35)c3ccc4c(c3)c3ccccc3n4-c3ccccc3)cc2)cc1] 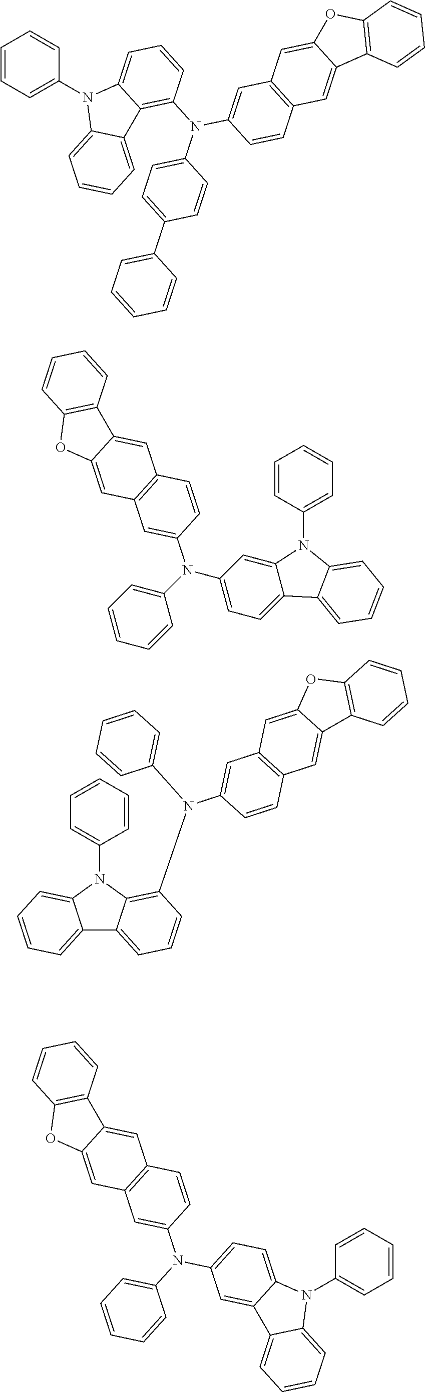 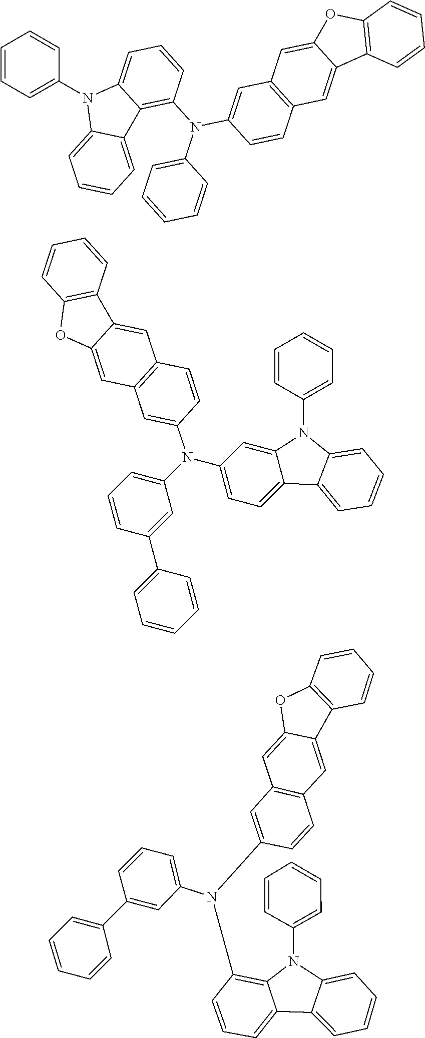 [SMILES: c1ccc(N(c2ccc3cc4c(cc3c2)oc2ccccc24)c2cccc3c2c2ccccc2n3-c2ccccc2)cc1 ; c1ccc(-c2cccc(N(c3ccc4cc5c(cc4c3)oc3ccccc35)c3ccc4c5ccccc5n(-c5ccccc5)c4c3)c2)cc1 ; c1ccc(-c2cccc(N(c3ccc4cc5c(cc4c3)oc3ccccc35)c3cccc4c5ccccc5n(-c5ccccc5)c34)c2)cc1] 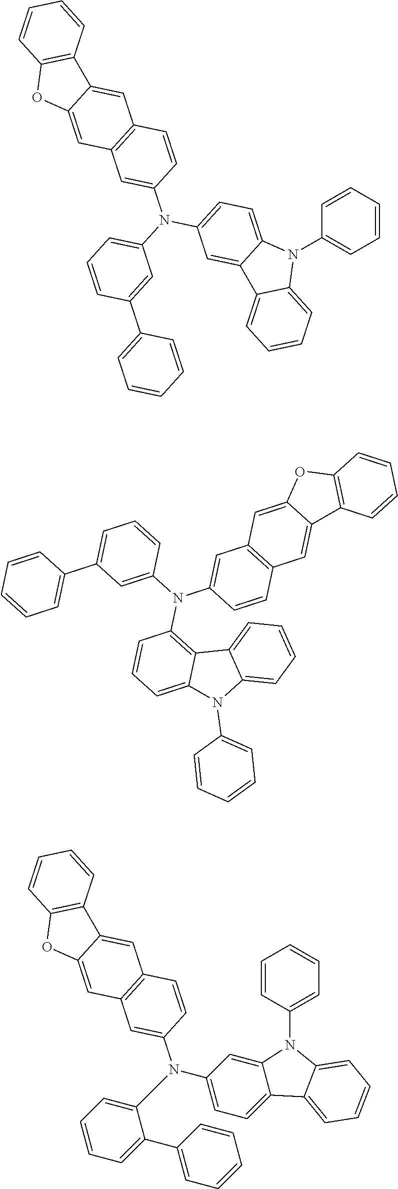 [SMILES: c1ccc(-c2cccc(N(c3ccc4cc5c(cc4c3)oc3ccccc35)c3ccc4c(c3)c3ccccc3n4-c3ccccc3)c2)cc1 ; c1ccc(-c2cccc(N(c3ccc4cc5c(cc4c3)oc3ccccc35)c3cccc4c3c3ccccc3n4-c3ccccc3)c2)cc1 ; c1ccc(-c2ccccc2N(c2ccc3cc4c(cc3c2)oc2ccccc24)c2ccc3c4ccccc4n(-c4ccccc4)c3c2)cc1] 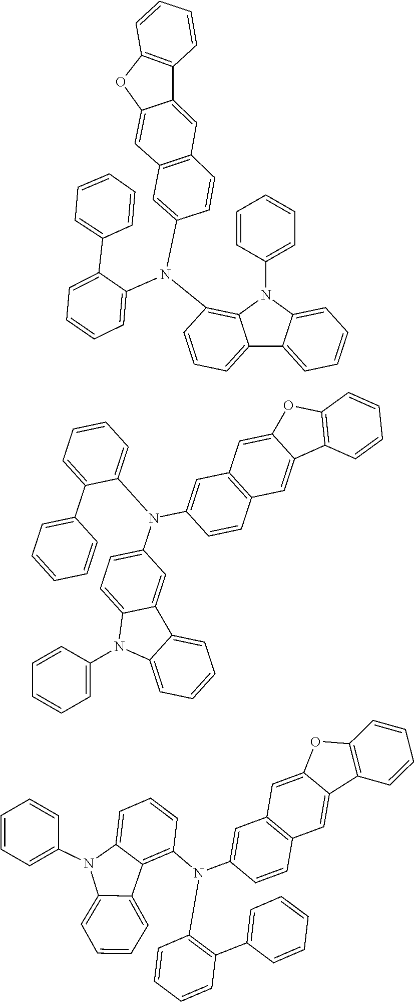 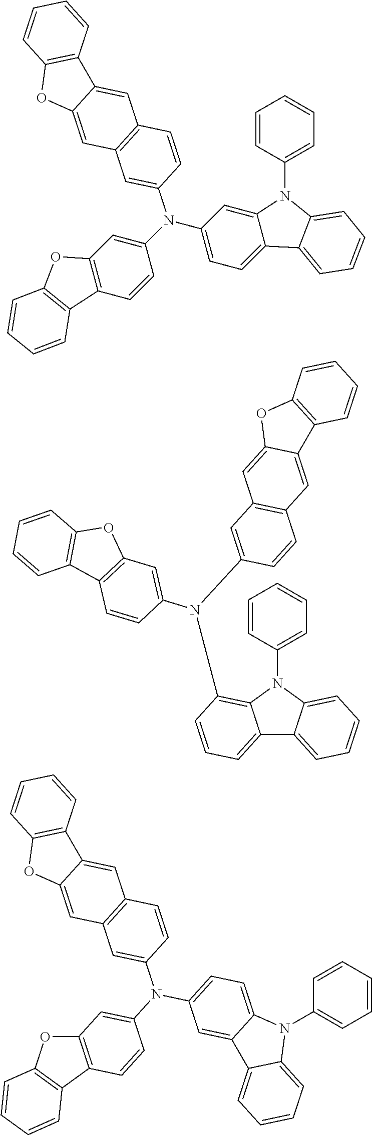 [SMILES: c1ccc(-n2c3ccccc3c3ccc(N(c4ccc5cc6c(cc5c4)oc4ccccc46)c4ccc5c(c4)oc4ccccc45)cc32)cc1 ; c1ccc(-n2c3ccccc3c3cccc(N(c4ccc5cc6c(cc5c4)oc4ccccc46)c4ccc5c(c4)oc4ccccc45)c32)cc1 ; c1ccc(-n2c3ccccc3c3cc(N(c4ccc5cc6c(cc5c4)oc4ccccc46)c4ccc5c(c4)oc4ccccc45)ccc32)cc1] 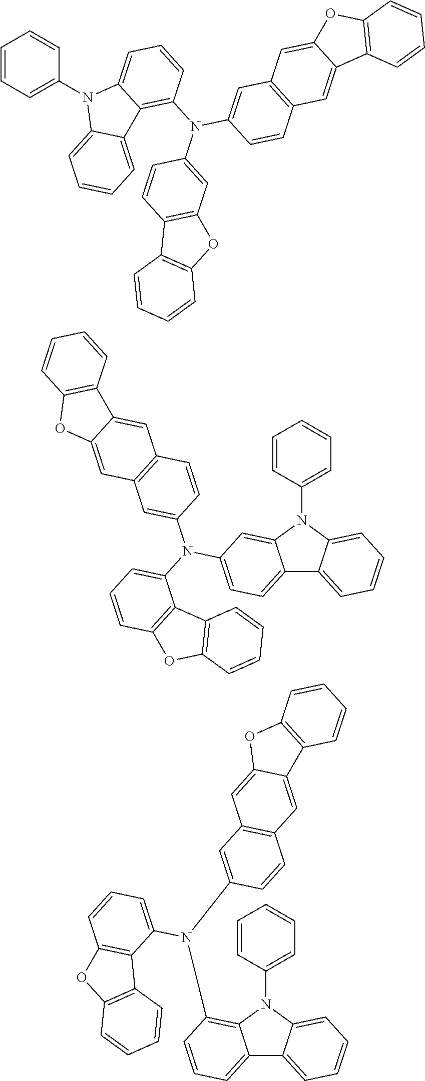 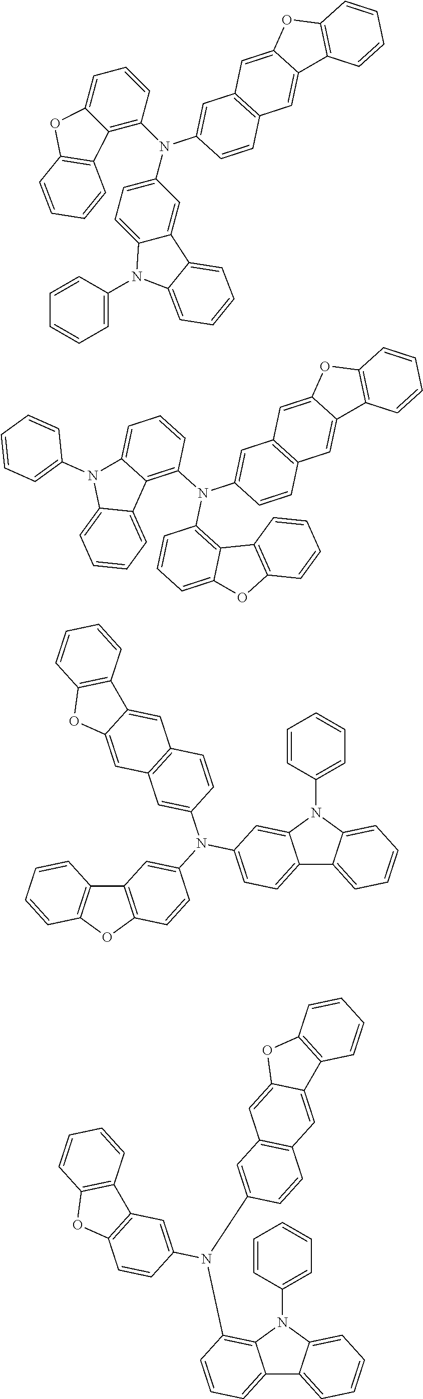 [SMILES: c1ccc(-n2c3ccccc3c3cc(N(c4ccc5cc6c(cc5c4)oc4ccccc46)c4cccc5oc6ccccc6c45)ccc32)cc1 ; c1ccc(-n2c3ccccc3c3c(N(c4ccc5cc6c(cc5c4)oc4ccccc46)c4cccc5oc6ccccc6c45)cccc32)cc1 ; c1ccc(-n2c3ccccc3c3ccc(N(c4ccc5cc6c(cc5c4)oc4ccccc46)c4ccc5oc6ccccc6c5c4)cc32)cc1 ; c1ccc(-n2c3ccccc3c3cccc(N(c4ccc5cc6c(cc5c4)oc4ccccc46)c4ccc5oc6ccccc6c5c4)c32)cc1] 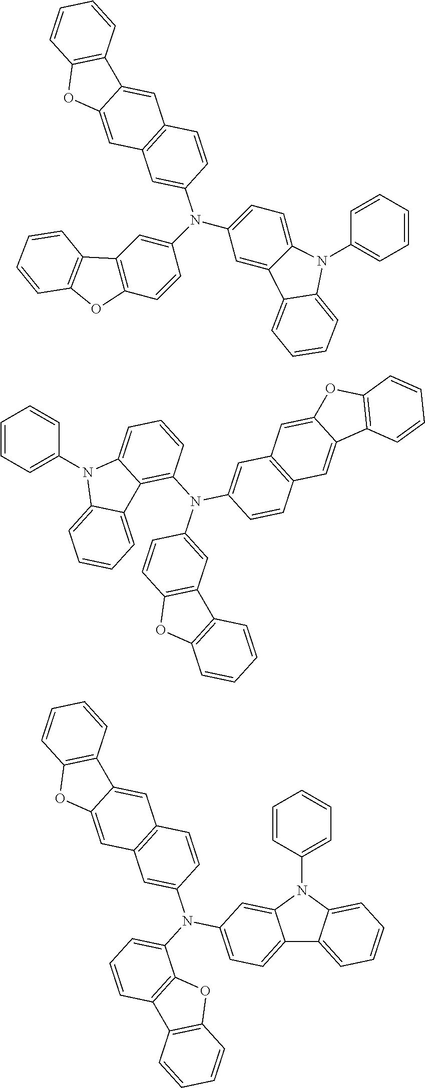 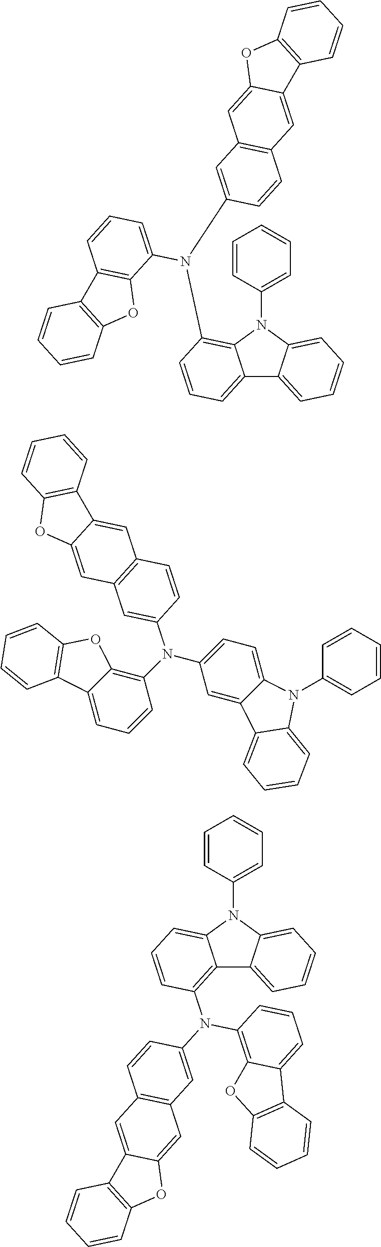 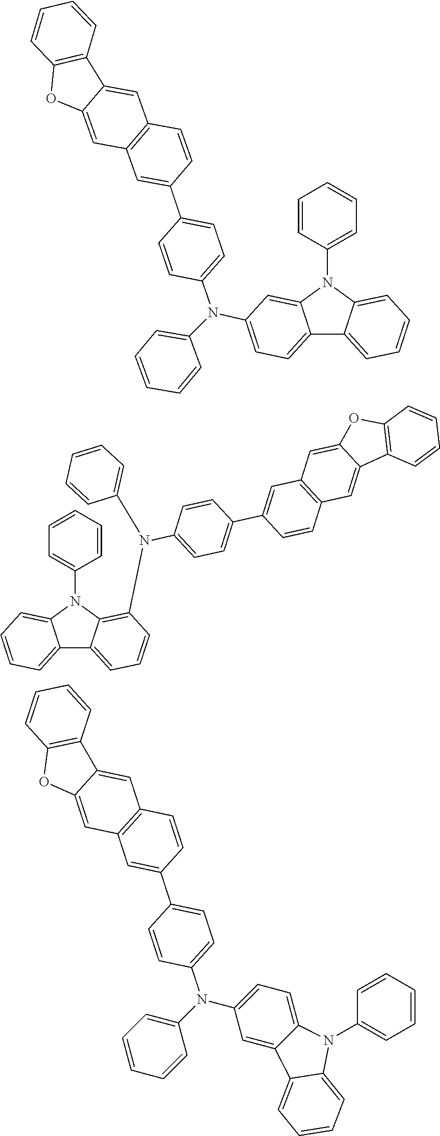 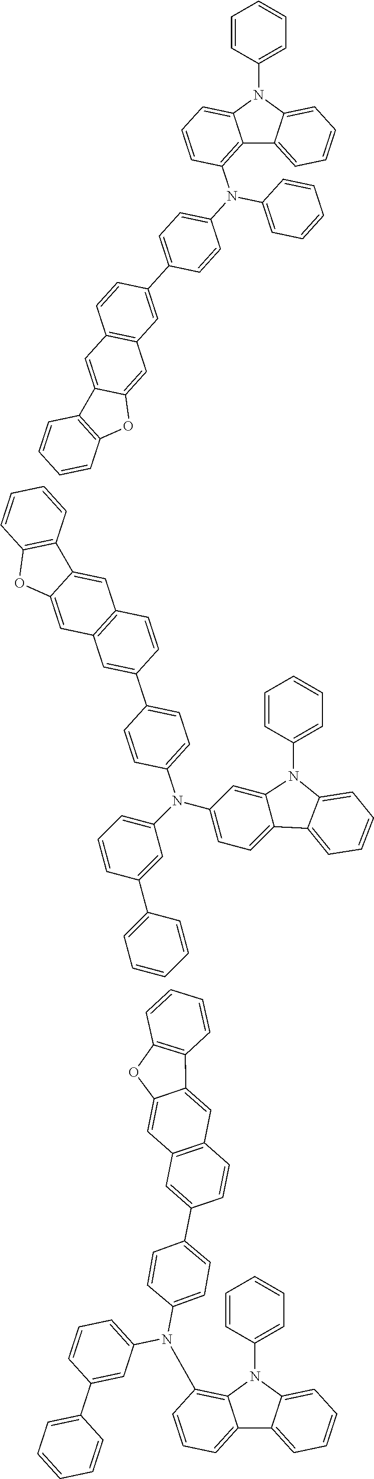 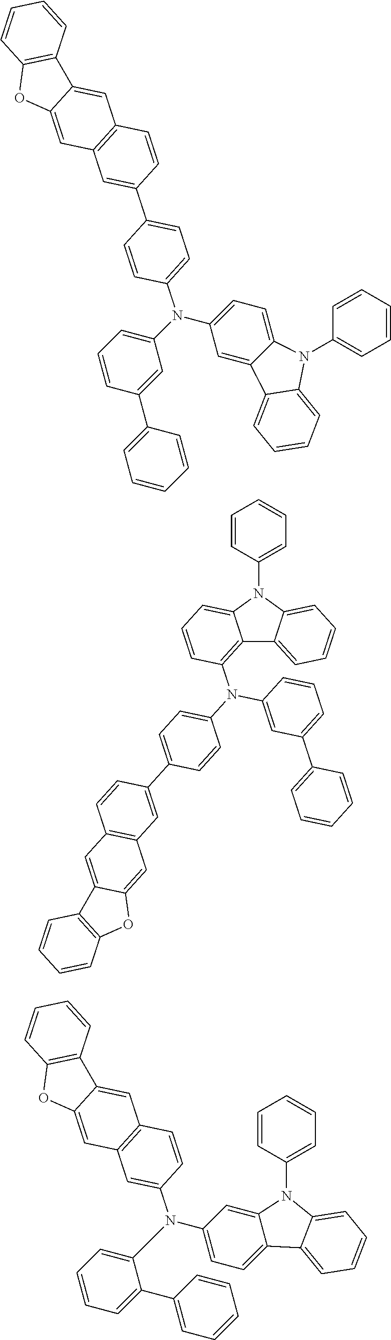 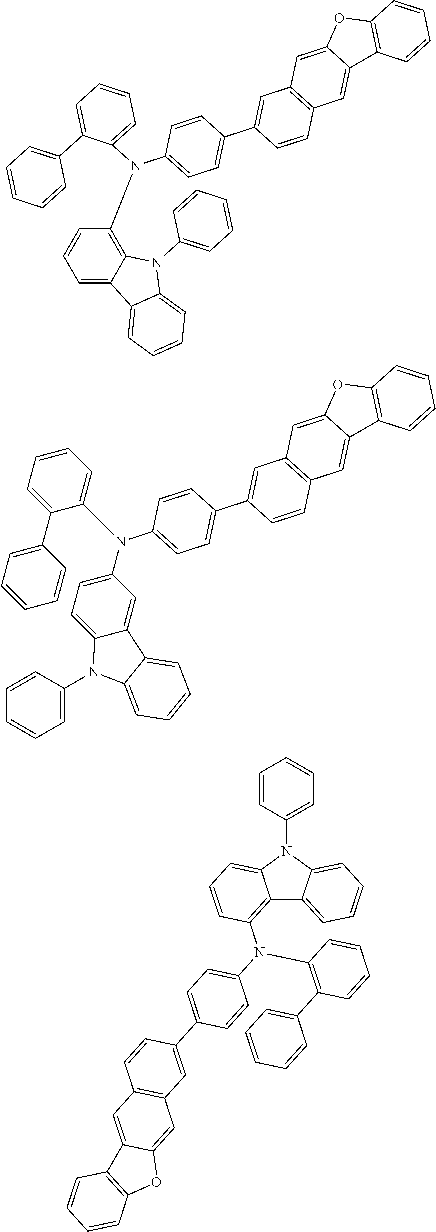 [SMILES: c1ccc(-c2ccccc2N(c2ccc(-c3ccc4cc5c(cc4c3)oc3ccccc35)cc2)c2cccc3c4ccccc4n(-c4ccccc4)c23)cc1 ; c1ccc(-c2ccccc2N(c2ccc(-c3ccc4cc5c(cc4c3)oc3ccccc35)cc2)c2ccc3c(c2)c2ccccc2n3-c2ccccc2)cc1 ; c1ccc(-c2ccccc2N(c2ccc(-c3ccc4cc5c(cc4c3)oc3ccccc35)cc2)c2cccc3c2c2ccccc2n3-c2ccccc2)cc1] 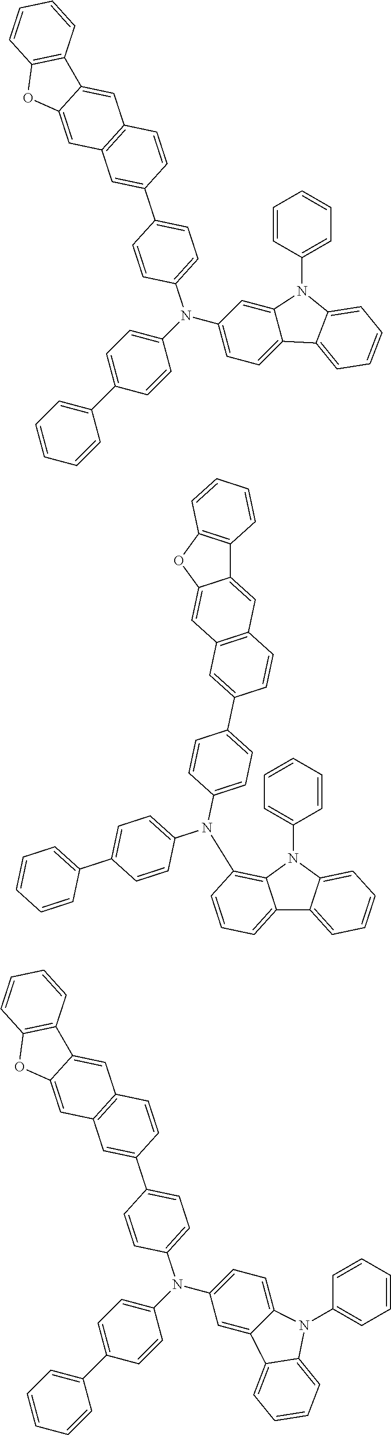 [SMILES: c1ccc(-c2ccc(N(c3ccc(-c4ccc5cc6c(cc5c4)oc4ccccc46)cc3)c3ccc4c5ccccc5n(-c5ccccc5)c4c3)cc2)cc1 ; c1ccc(-c2ccc(N(c3ccc(-c4ccc5cc6c(cc5c4)oc4ccccc46)cc3)c3cccc4c5ccccc5n(-c5ccccc5)c34)cc2)cc1 ; c1ccc(-c2ccc(N(c3ccc(-c4ccc5cc6c(cc5c4)oc4ccccc46)cc3)c3ccc4c(c3)c3ccccc3n4-c3ccccc3)cc2)cc1] 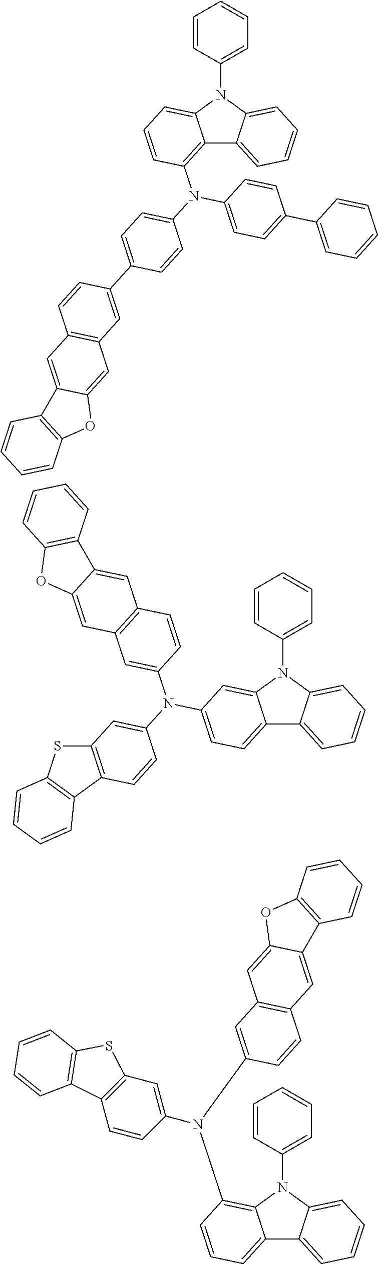 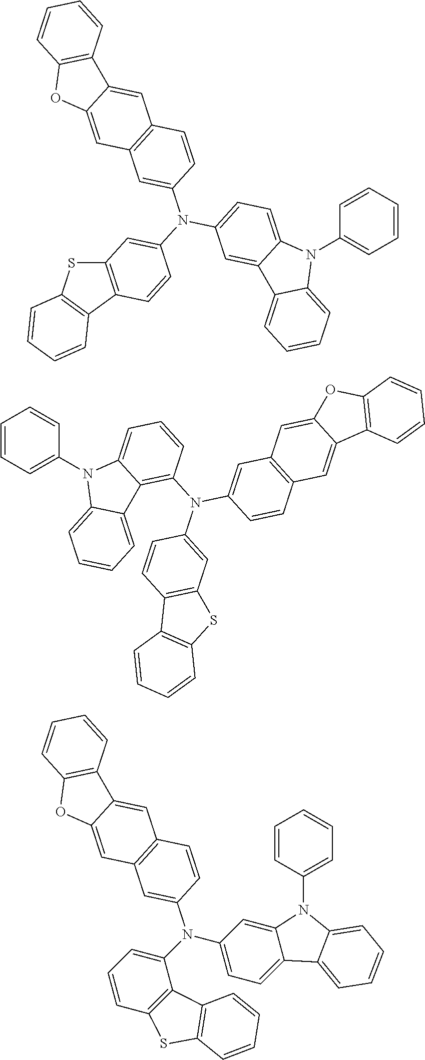 [SMILES: c1ccc(-n2c3ccccc3c3cc(N(c4ccc5cc6c(cc5c4)oc4ccccc46)c4ccc5c(c4)sc4ccccc45)ccc32)cc1 ; c1ccc(-n2c3ccccc3c3c(N(c4ccc5cc6c(cc5c4)oc4ccccc46)c4ccc5c(c4)sc4ccccc45)cccc32)cc1 ; c1ccc(-n2c3ccccc3c3ccc(N(c4ccc5cc6c(cc5c4)oc4ccccc46)c4cccc5sc6ccccc6c45)cc32)cc1] 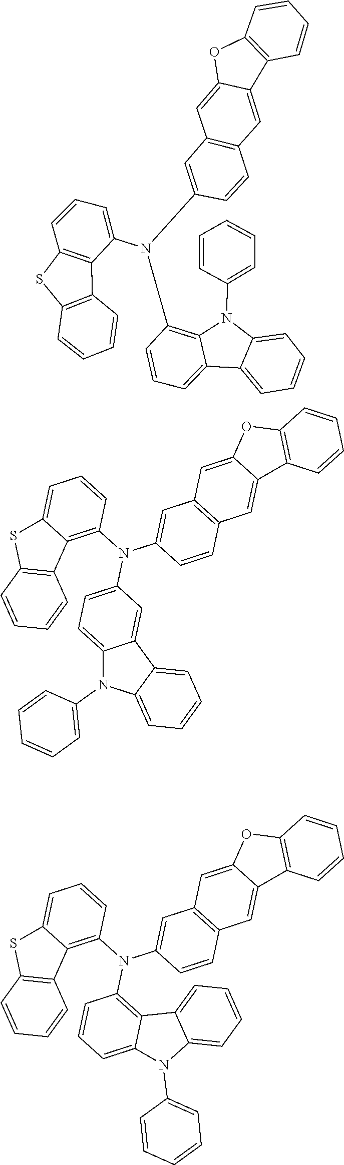 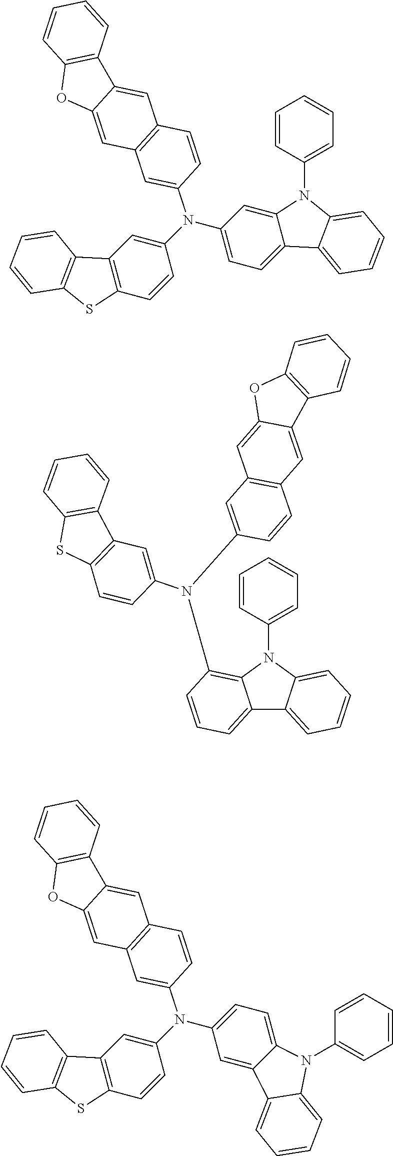 [SMILES: c1ccc(-n2c3ccccc3c3ccc(N(c4ccc5cc6c(cc5c4)oc4ccccc46)c4ccc5sc6ccccc6c5c4)cc32)cc1 ; c1ccc(-n2c3ccccc3c3cccc(N(c4ccc5cc6c(cc5c4)oc4ccccc46)c4ccc5sc6ccccc6c5c4)c32)cc1 ; c1ccc(-n2c3ccccc3c3cc(N(c4ccc5cc6c(cc5c4)oc4ccccc46)c4ccc5sc6ccccc6c5c4)ccc32)cc1] 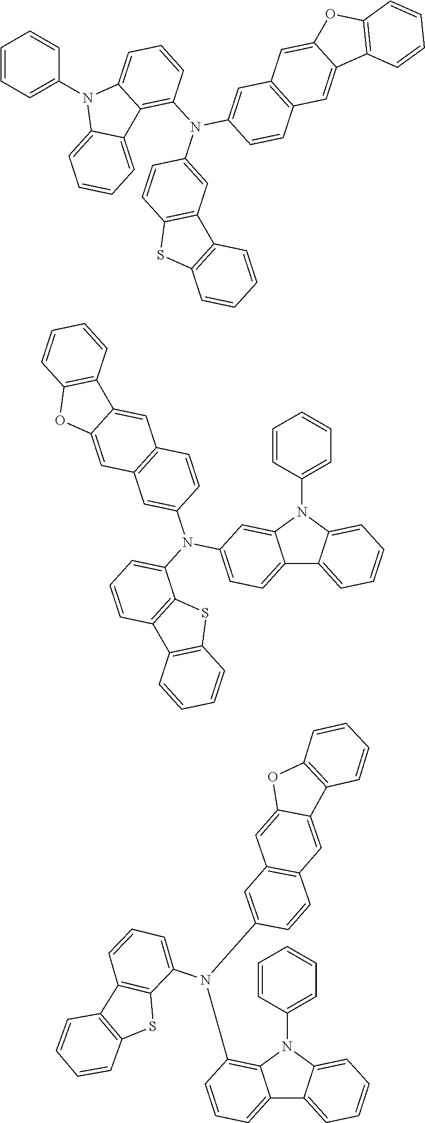 [SMILES: c1ccc(-n2c3ccccc3c3c(N(c4ccc5cc6c(cc5c4)oc4ccccc46)c4ccc5sc6ccccc6c5c4)cccc32)cc1 ; c1ccc(-n2c3ccccc3c3ccc(N(c4ccc5cc6c(cc5c4)oc4ccccc46)c4cccc5c4sc4ccccc45)cc32)cc1 ; c1ccc(-n2c3ccccc3c3cccc(N(c4ccc5cc6c(cc5c4)oc4ccccc46)c4cccc5c4sc4ccccc45)c32)cc1] 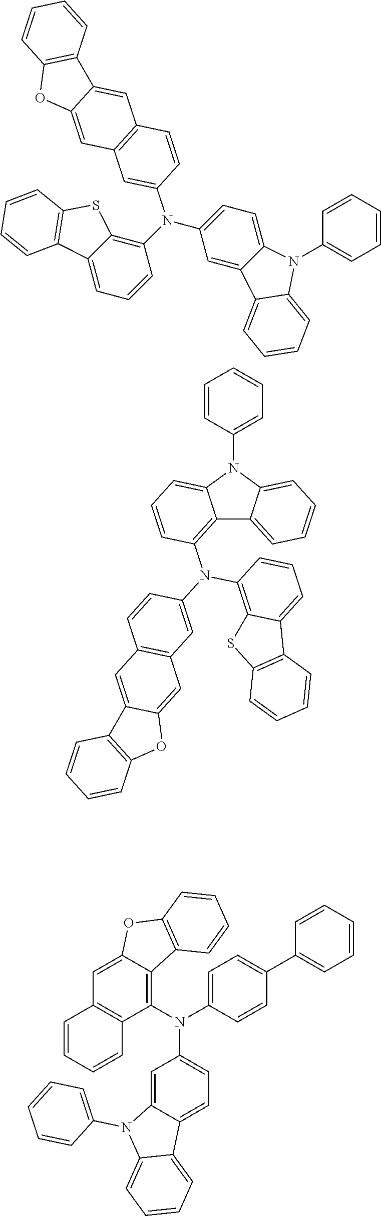 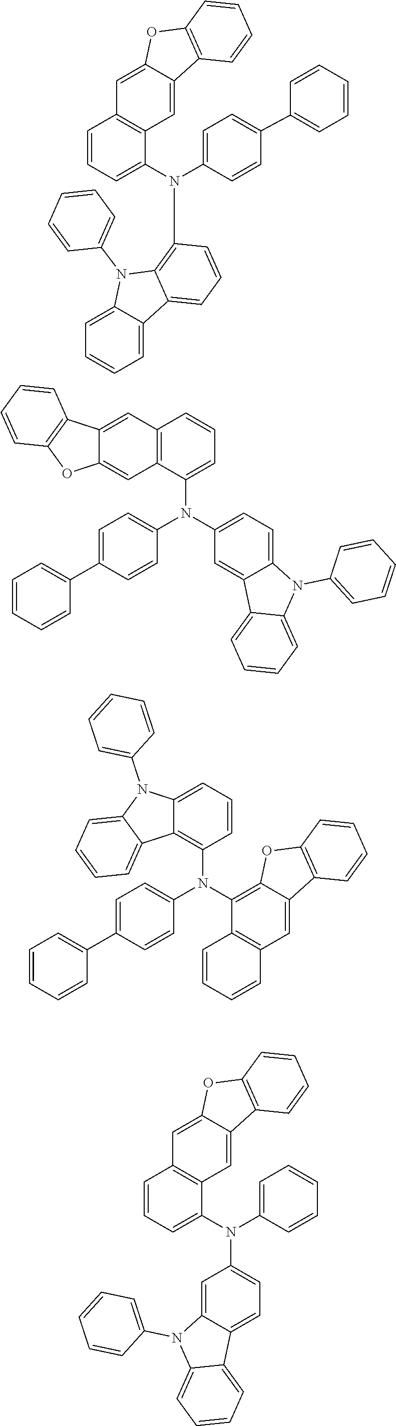 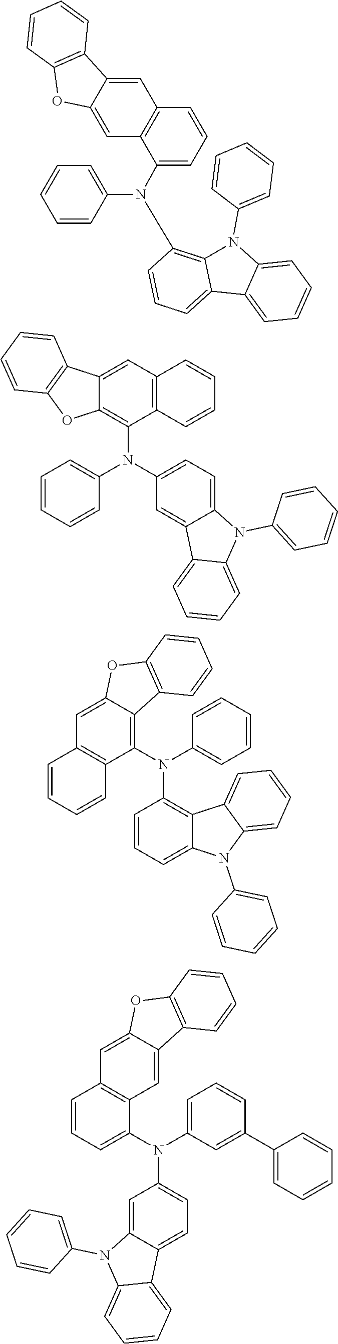 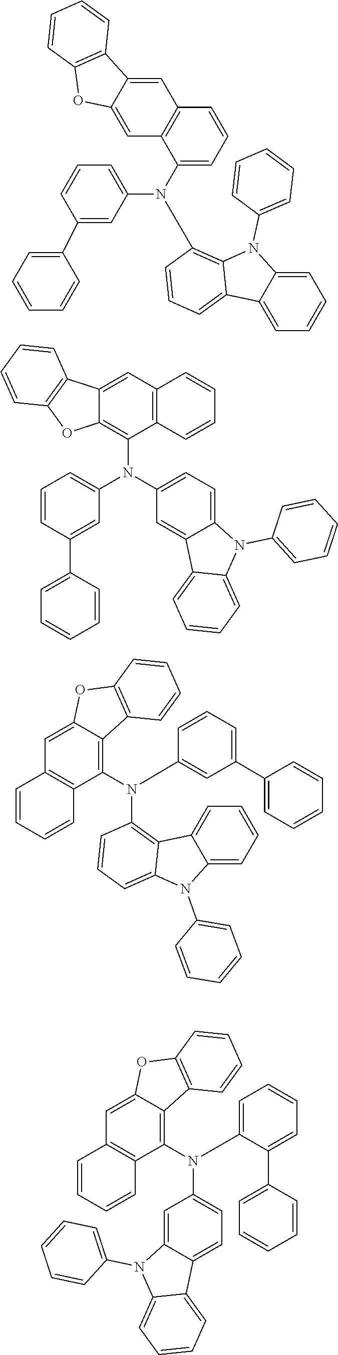 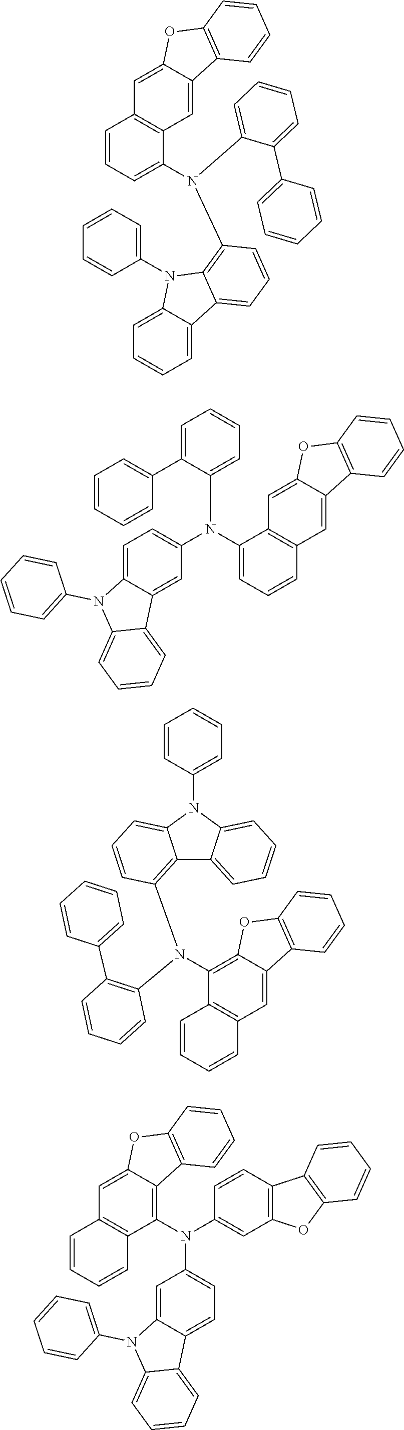 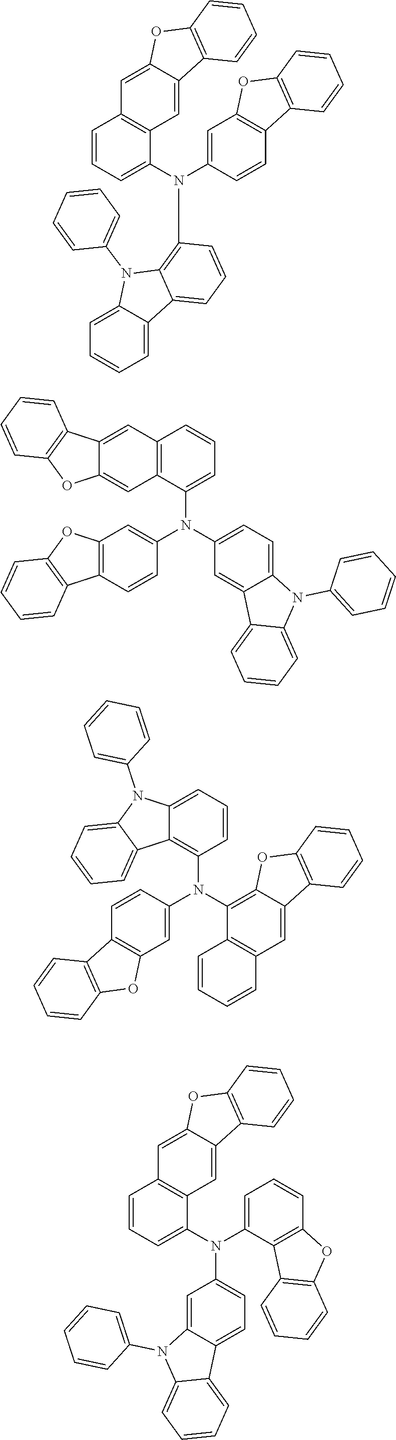 [SMILES: c1ccc(-n2c3ccccc3c3cccc(N(c4ccc5c(c4)oc4ccccc45)c4cccc5cc6oc7ccccc7c6cc45)c32)cc1 ; c1ccc(-n2c3ccccc3c3cc(N(c4ccc5c(c4)oc4ccccc45)c4cccc5cc6c(cc45)oc4ccccc46)ccc32)cc1 ; c1ccc(-n2c3ccccc3c3c(N(c4ccc5c(c4)oc4ccccc45)c4c5ccccc5cc5c4oc4ccccc45)cccc32)cc1 ; c1ccc(-n2c3ccccc3c3ccc(N(c4cccc5cc6oc7ccccc7c6cc45)c4cccc5oc6ccccc6c45)cc32)cc1] 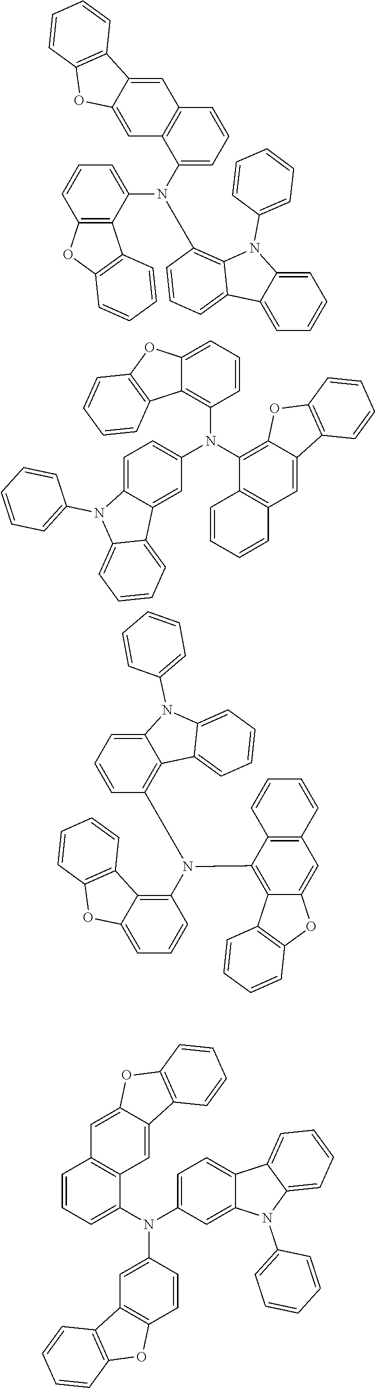 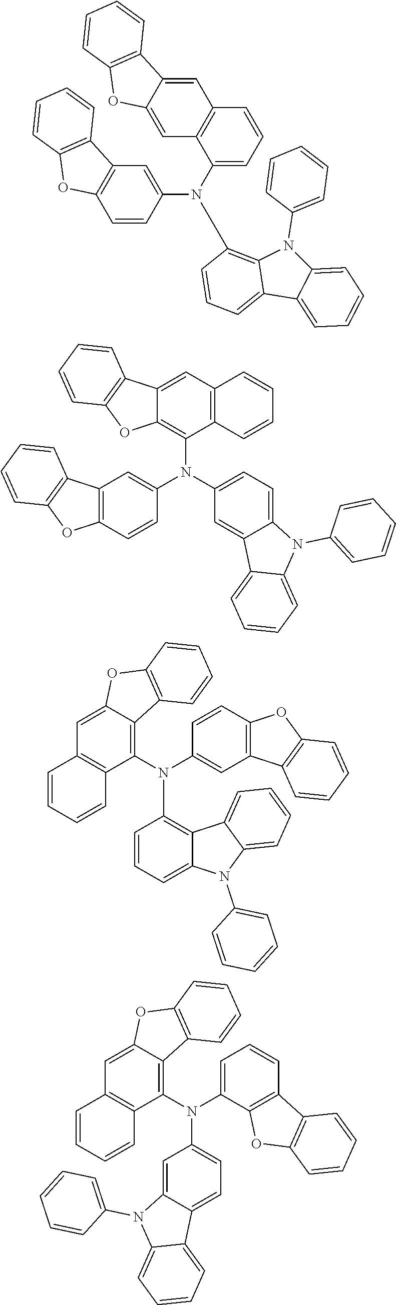 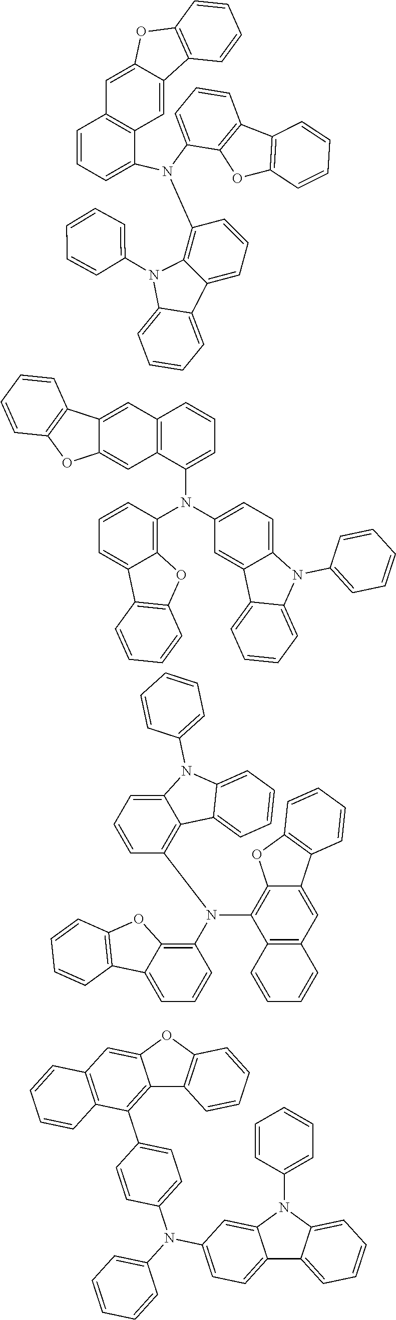 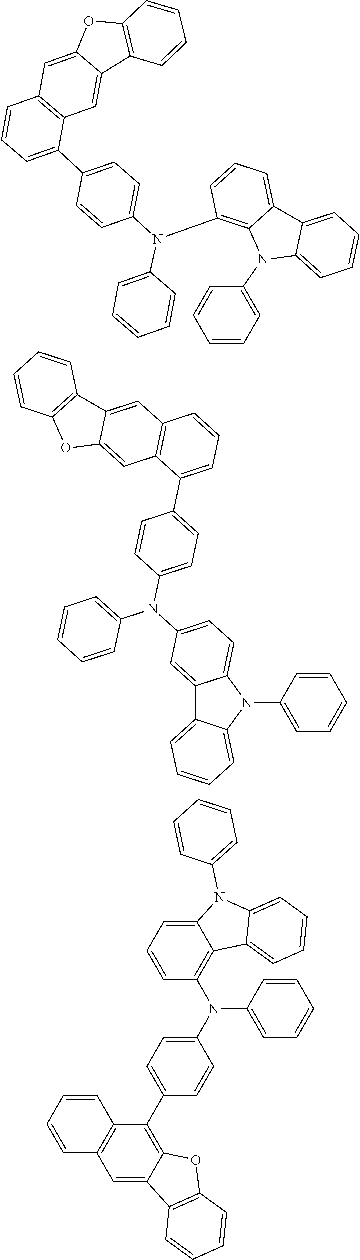 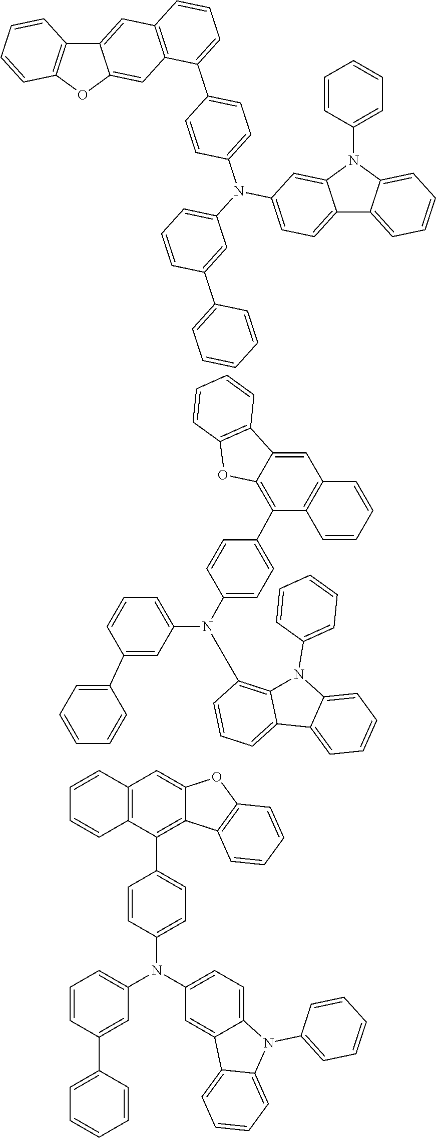 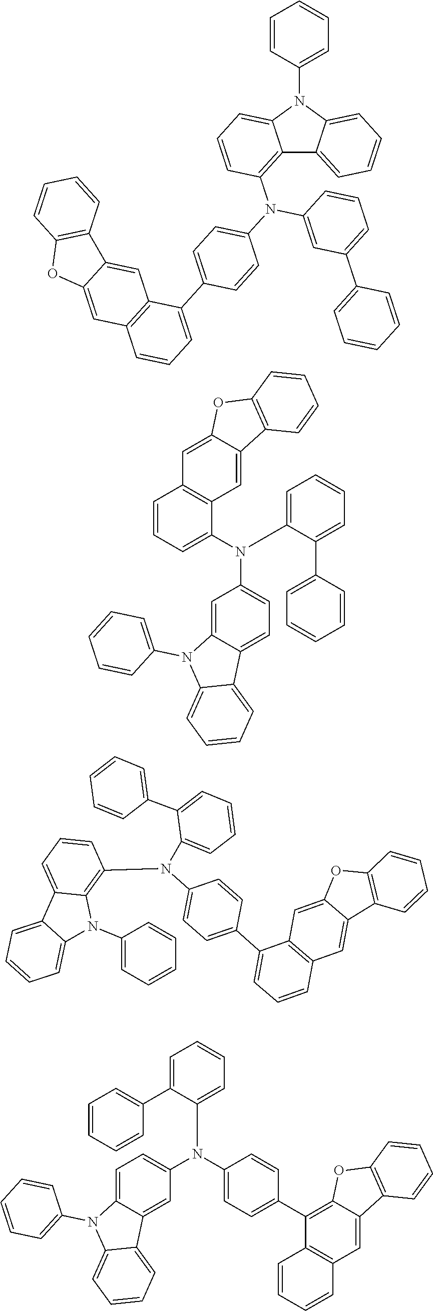 [SMILES: c1ccc(-c2cccc(N(c3ccc(-c4cccc5cc6oc7ccccc7c6cc45)cc3)c3cccc4c3c3ccccc3n4-c3ccccc3)c2)cc1 ; c1ccc(-c2ccccc2N(c2ccc3c4ccccc4n(-c4ccccc4)c3c2)c2cccc3cc4oc5ccccc5c4cc23)cc1 ; c1ccc(-c2ccccc2N(c2ccc(-c3cccc4cc5c(cc34)oc3ccccc35)cc2)c2cccc3c4ccccc4n(-c4ccccc4)c23)cc1 ; c1ccc(-c2ccccc2N(c2ccc(-c3c4ccccc4cc4c3oc3ccccc34)cc2)c2ccc3c(c2)c2ccccc2n3-c2ccccc2)cc1] 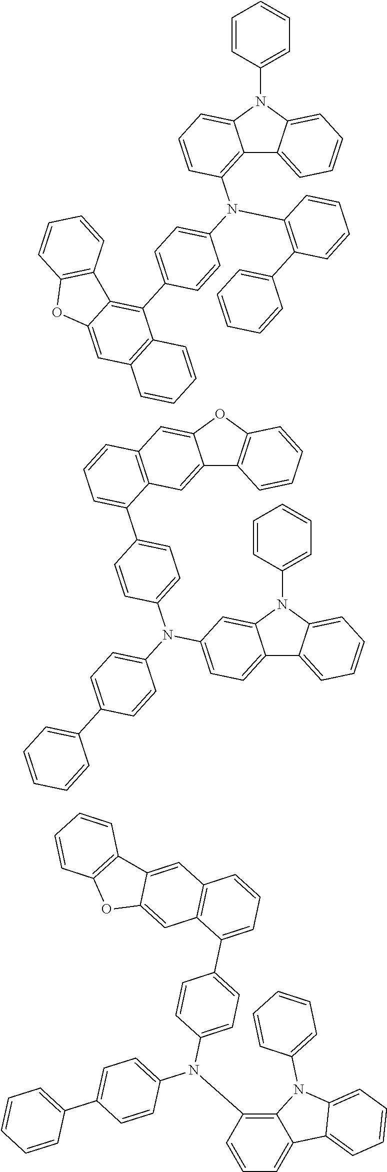 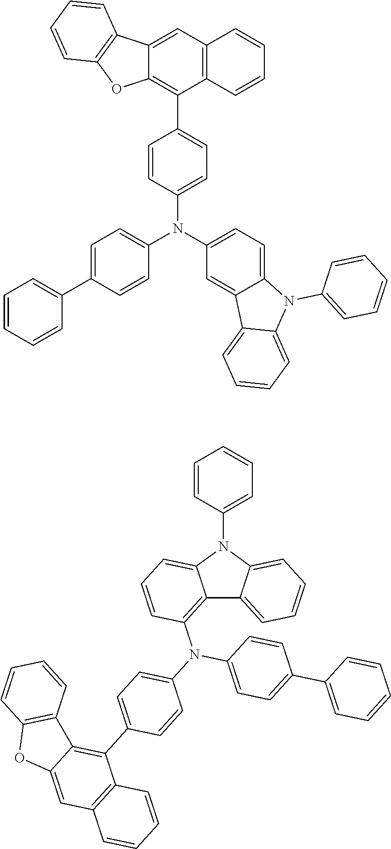 [SMILES: c1ccc(-c2ccc(N(c3ccc(-c4c5ccccc5cc5c4oc4ccccc45)cc3)c3ccc4c(c3)c3ccccc3n4-c3ccccc3)cc2)cc1 ; c1ccc(-c2ccc(N(c3ccc(-c4c5ccccc5cc5oc6ccccc6c45)cc3)c3cccc4c3c3ccccc3n4-c3ccccc3)cc2)cc1] 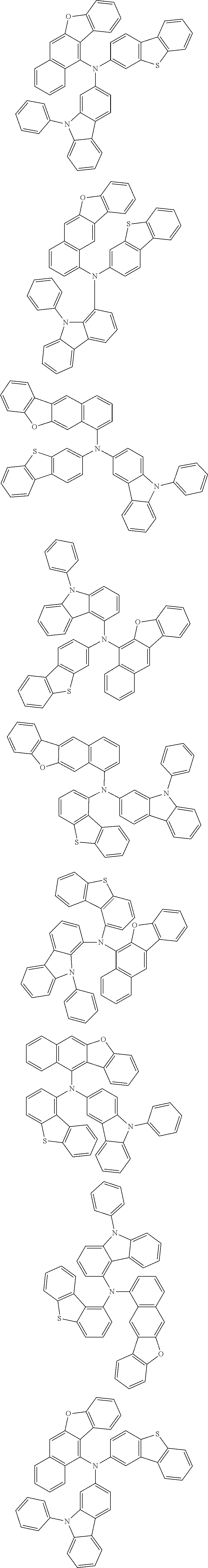 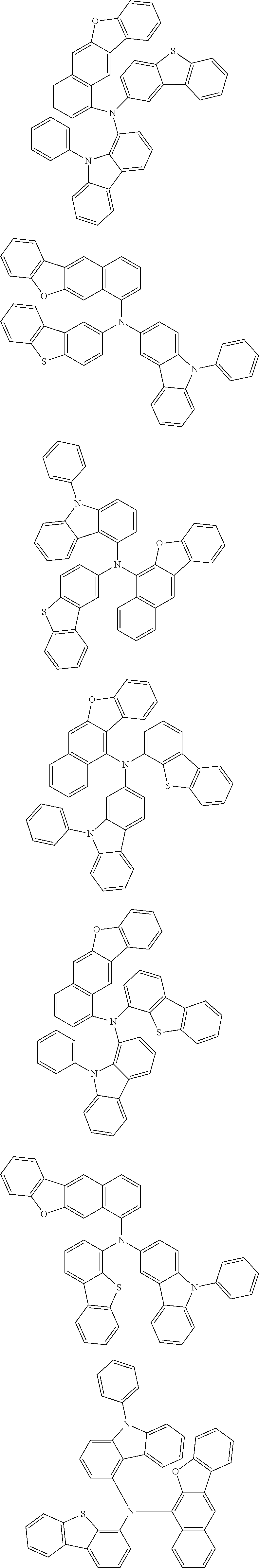 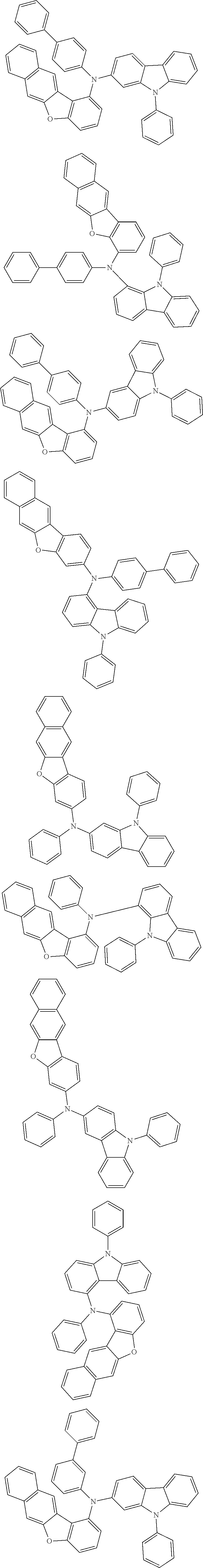 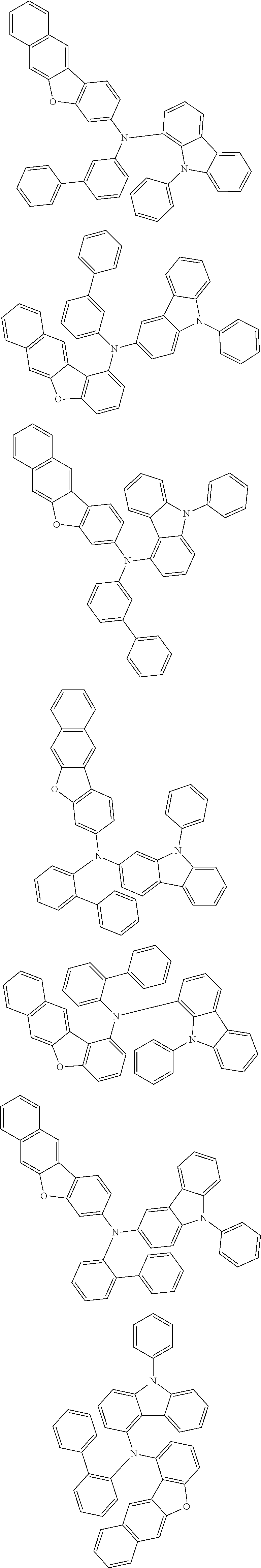 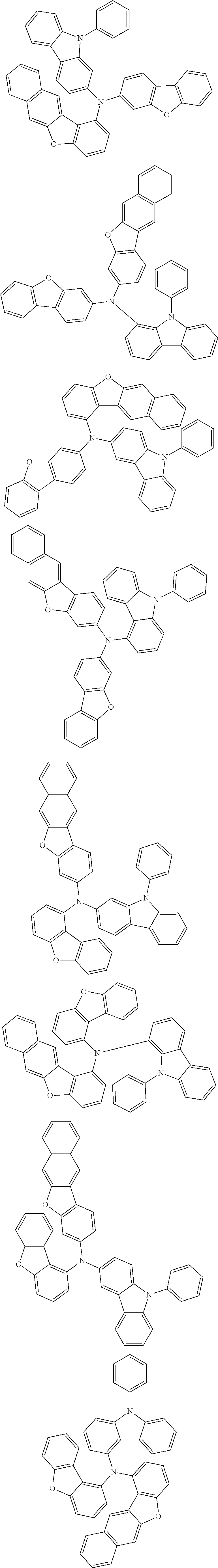 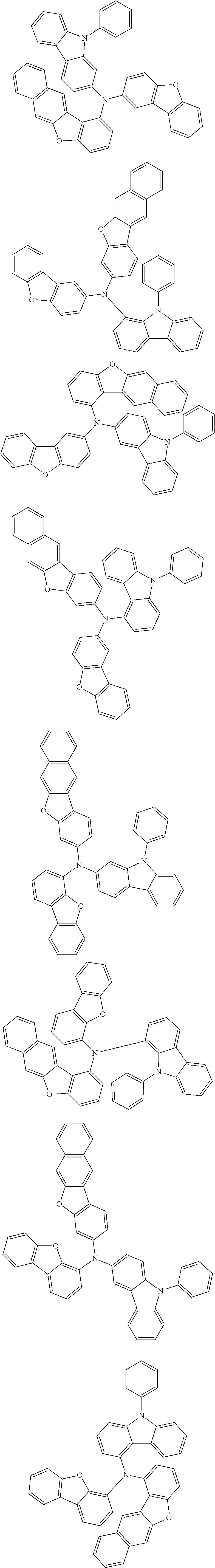 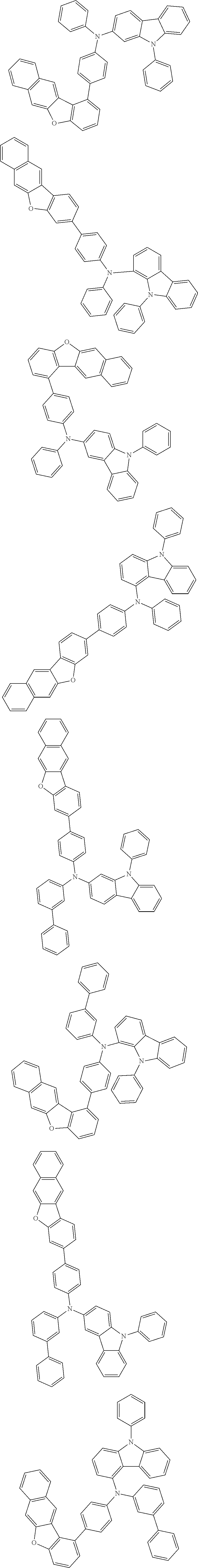 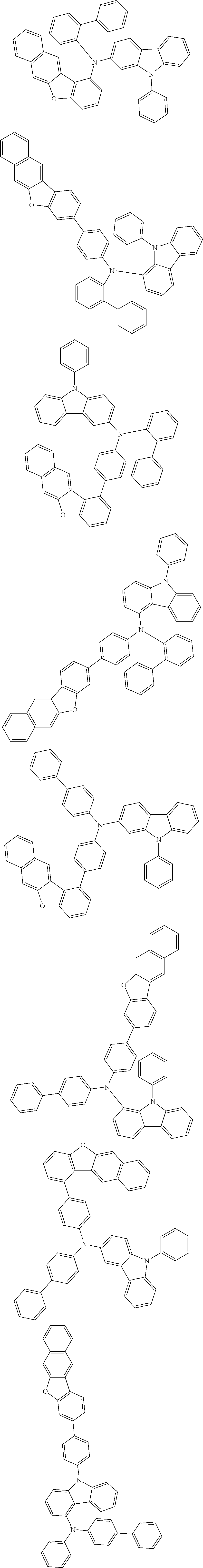 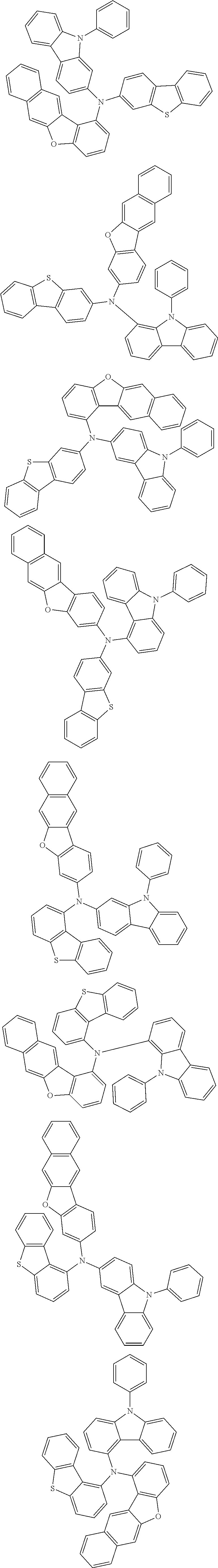 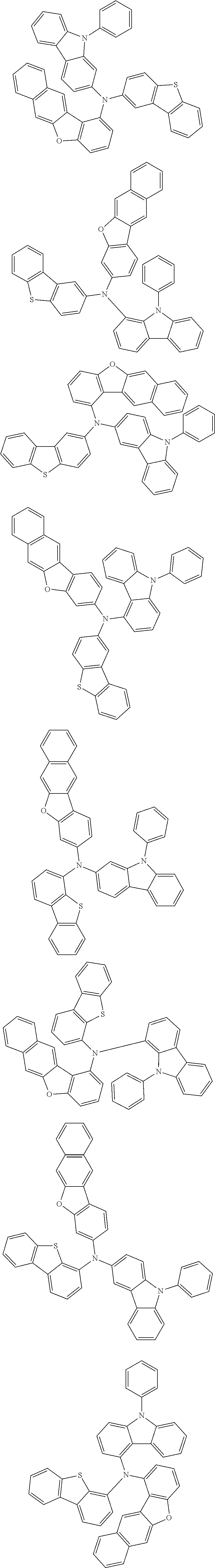 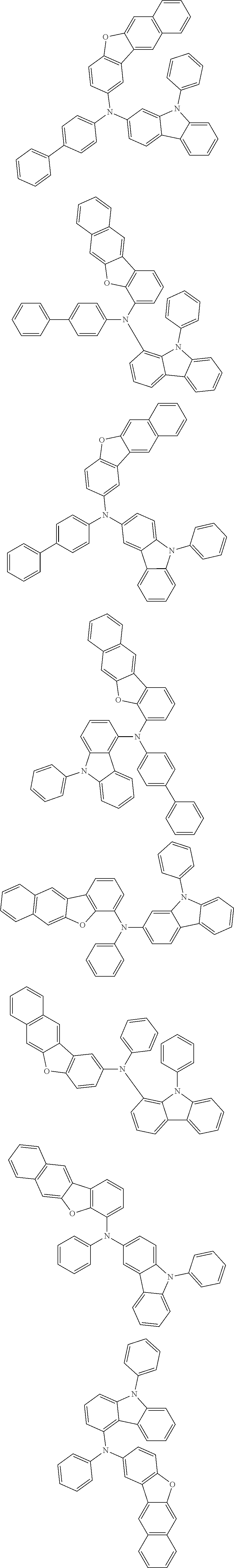 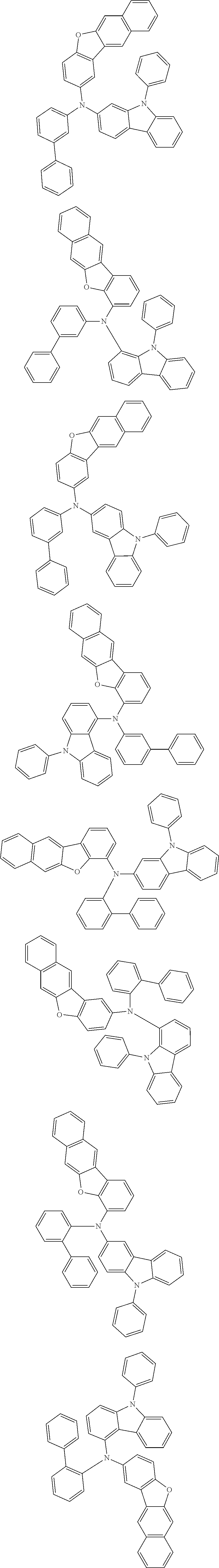 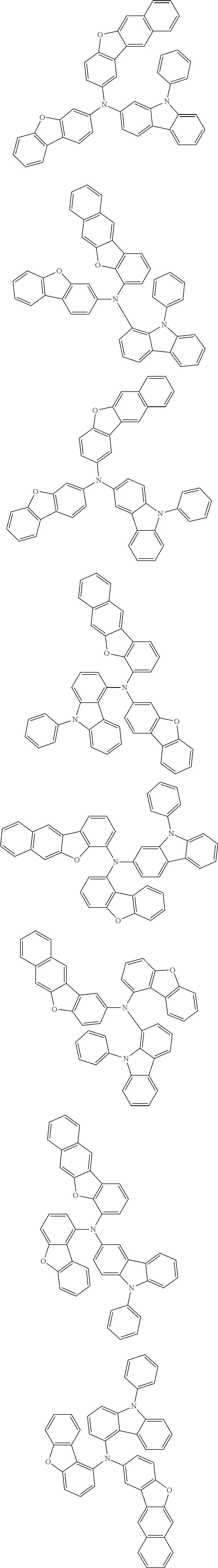 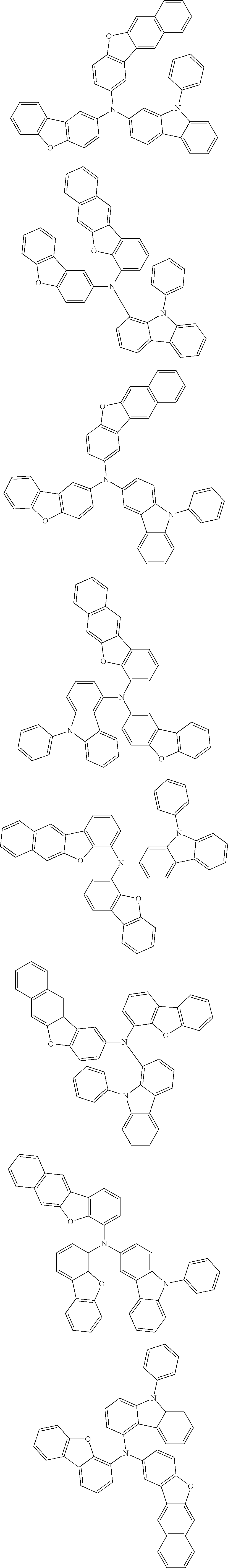 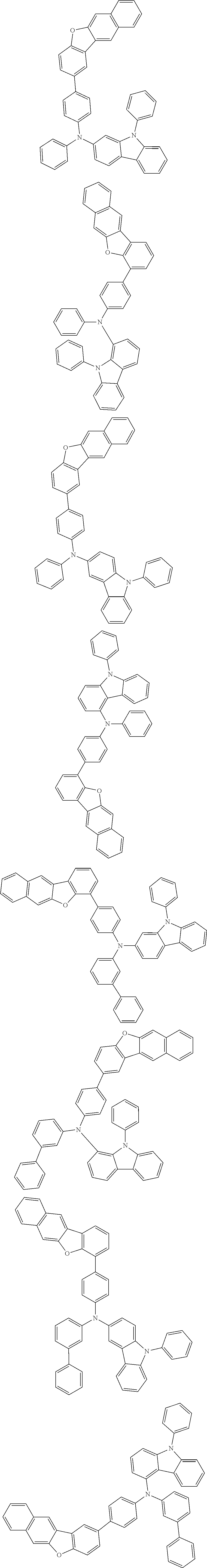 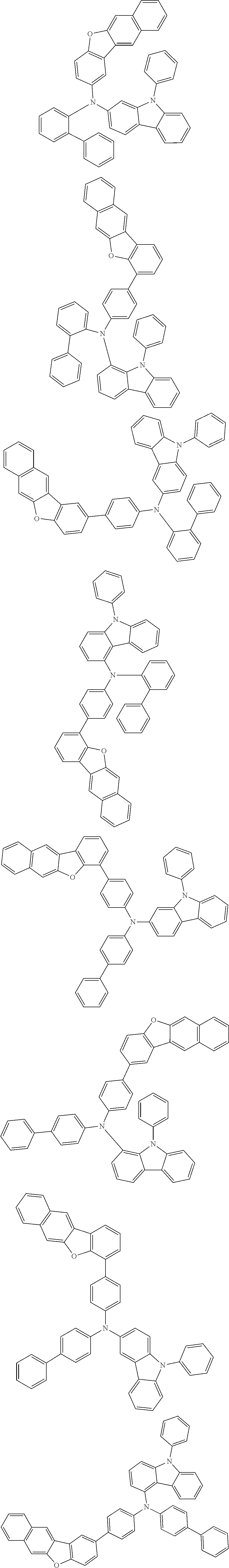 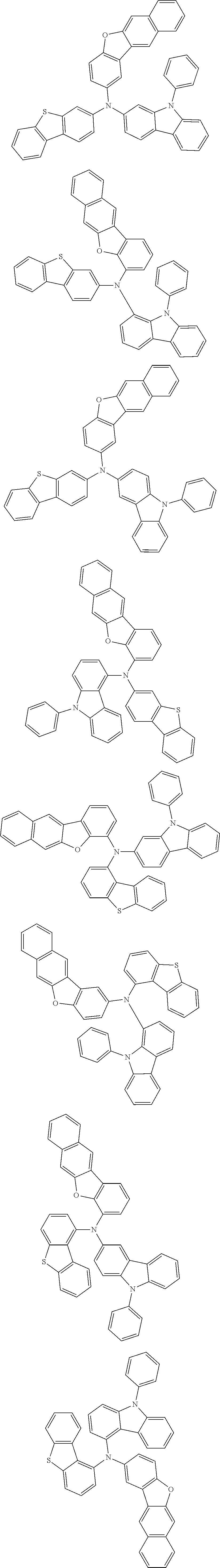 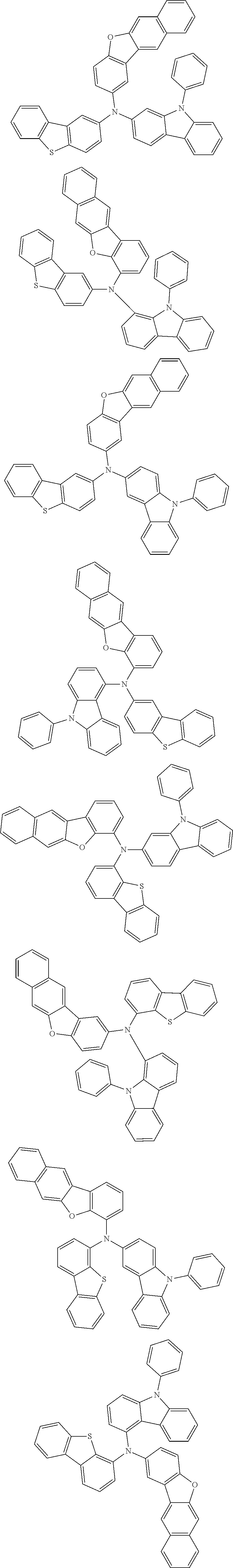 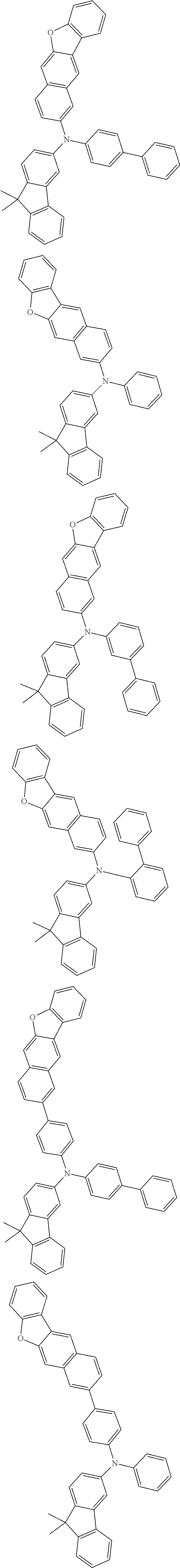 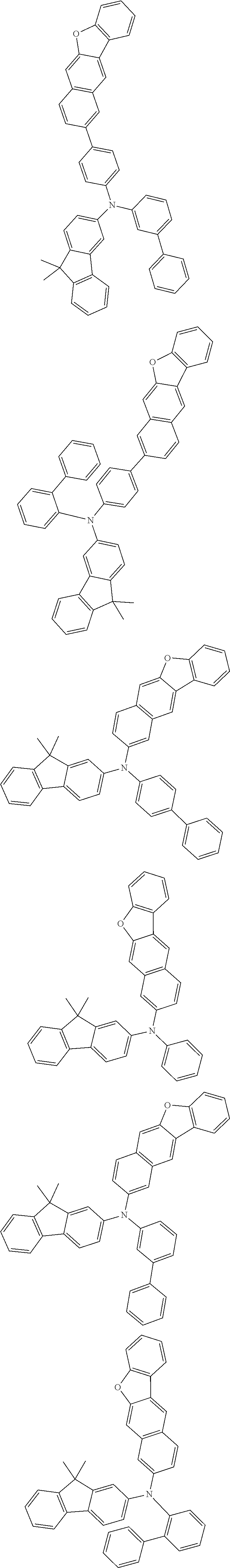 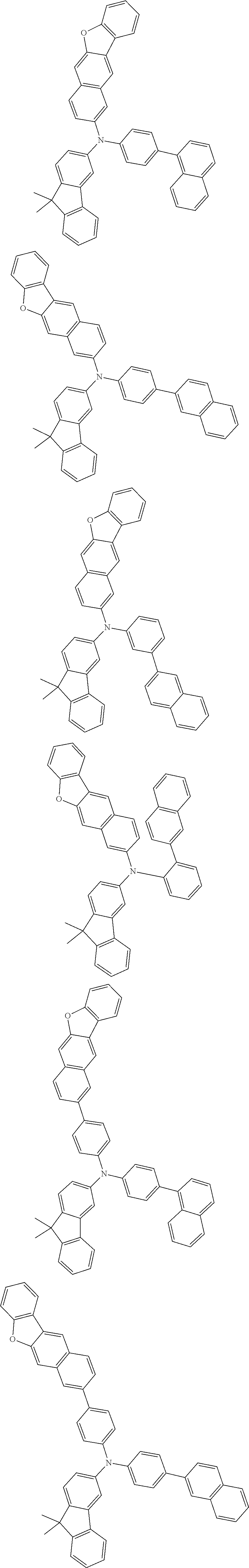 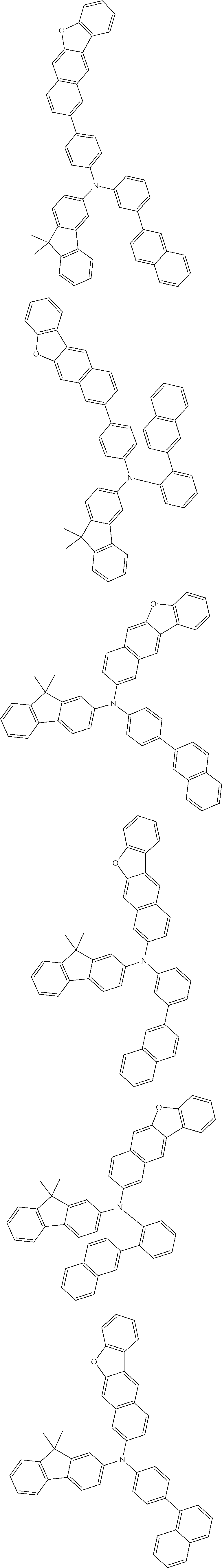 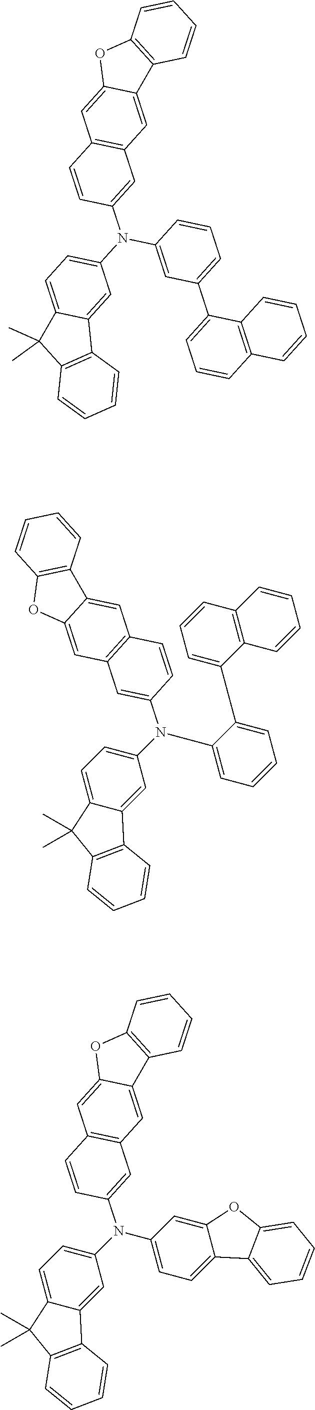 [SMILES: CC1(C)c2ccccc2-c2cc(N(c3cccc(-c4cccc5ccccc45)c3)c3ccc4cc5oc6ccccc6c5cc4c3)ccc21 ; CC1(C)c2ccccc2-c2cc(N(c3ccc4cc5c(cc4c3)oc3ccccc35)c3ccccc3-c3cccc4ccccc34)ccc21 ; CC1(C)c2ccccc2-c2cc(N(c3ccc4cc5oc6ccccc6c5cc4c3)c3ccc4c(c3)oc3ccccc34)ccc21] 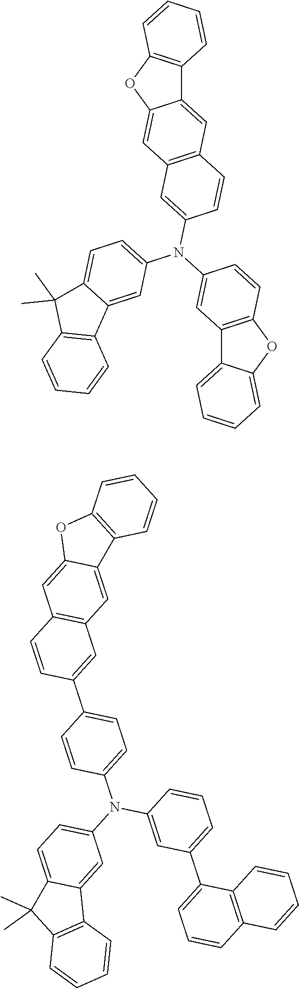 [SMILES: CC1(C)c2ccccc2-c2cc(N(c3ccc4cc5c(cc4c3)oc3ccccc35)c3ccc4oc5ccccc5c4c3)ccc21 ; CC1(C)c2ccccc2-c2cc(N(c3ccc(-c4ccc5cc6oc7ccccc7c6cc5c4)cc3)c3cccc(-c4cccc5ccccc45)c3)ccc21] 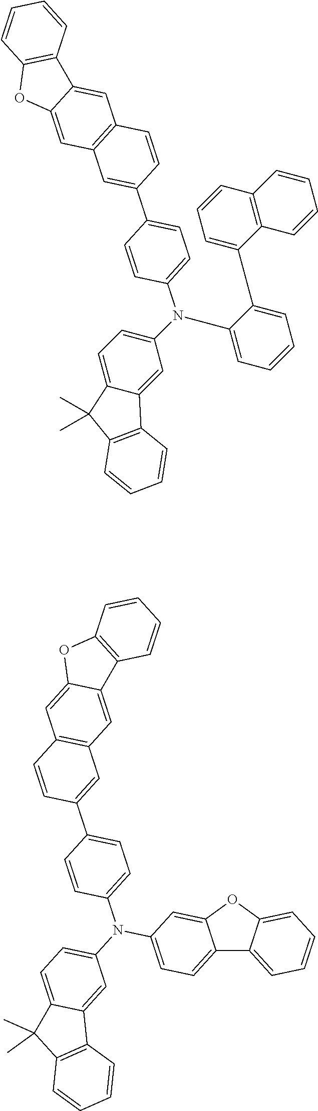 [SMILES: CC1(C)c2ccccc2-c2cc(N(c3ccc(-c4ccc5cc6c(cc5c4)oc4ccccc46)cc3)c3ccccc3-c3cccc4ccccc34)ccc21 ; CC1(C)c2ccccc2-c2cc(N(c3ccc(-c4ccc5cc6oc7ccccc7c6cc5c4)cc3)c3ccc4c(c3)oc3ccccc34)ccc21] 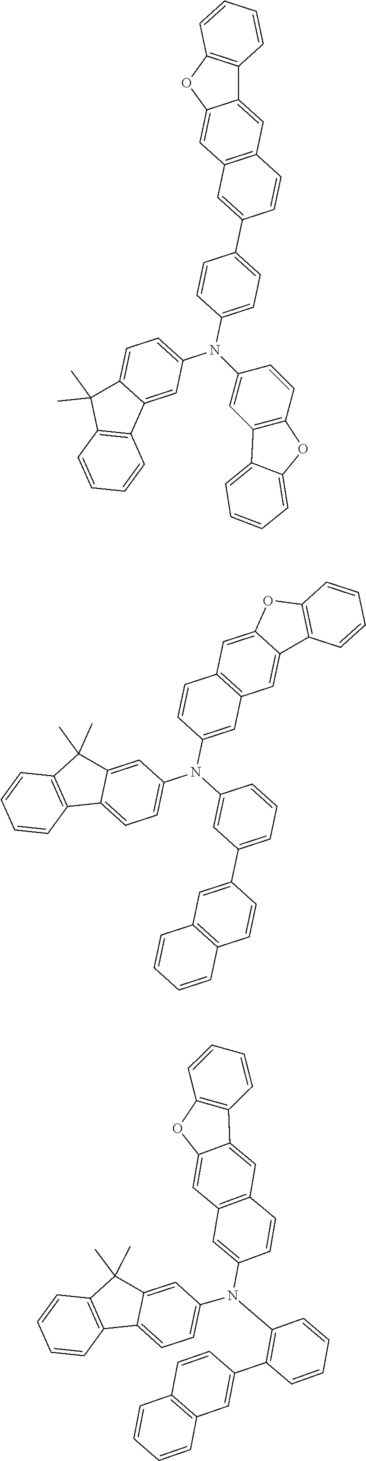 [SMILES: CC1(C)c2ccccc2-c2cc(N(c3ccc(-c4ccc5cc6c(cc5c4)oc4ccccc46)cc3)c3ccc4oc5ccccc5c4c3)ccc21 ; CC1(C)c2ccccc2-c2ccc(N(c3cccc(-c4ccc5ccccc5c4)c3)c3ccc4cc5oc6ccccc6c5cc4c3)cc21 ; CC1(C)c2ccccc2-c2ccc(N(c3ccc4cc5c(cc4c3)oc3ccccc35)c3ccccc3-c3ccc4ccccc4c3)cc21] 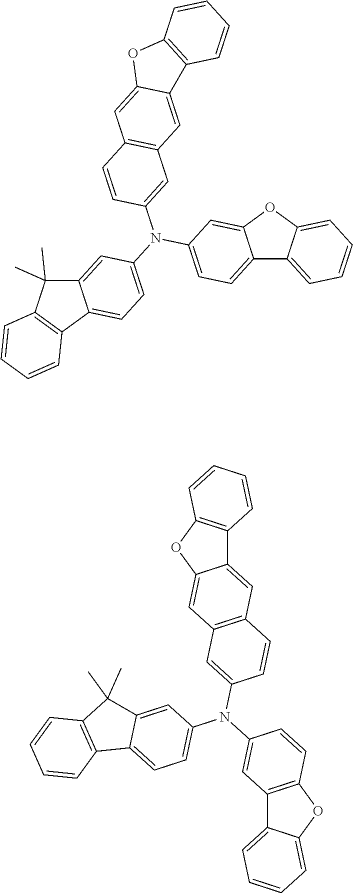 [SMILES: CC1(C)c2ccccc2-c2ccc(N(c3ccc4cc5oc6ccccc6c5cc4c3)c3ccc4c(c3)oc3ccccc34)cc21 ; CC1(C)c2ccccc2-c2ccc(N(c3ccc4cc5c(cc4c3)oc3ccccc35)c3ccc4oc5ccccc5c4c3)cc21] 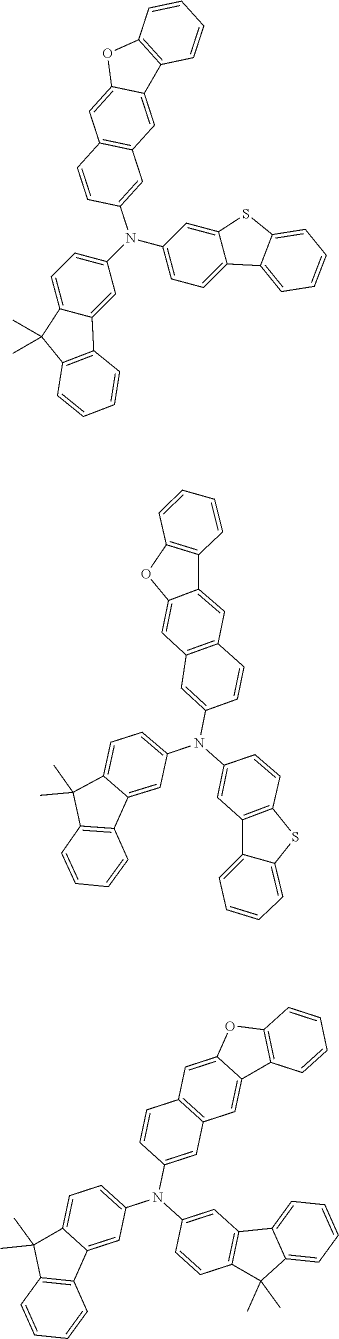 [SMILES: CC1(C)c2ccccc2-c2cc(N(c3ccc4cc5oc6ccccc6c5cc4c3)c3ccc4c(c3)sc3ccccc34)ccc21 ; CC1(C)c2ccccc2-c2cc(N(c3ccc4cc5c(cc4c3)oc3ccccc35)c3ccc4sc5ccccc5c4c3)ccc21 ; CC1(C)c2ccccc2-c2cc(N(c3ccc4c(c3)-c3ccccc3C4(C)C)c3ccc4cc5oc6ccccc6c5cc4c3)ccc21] 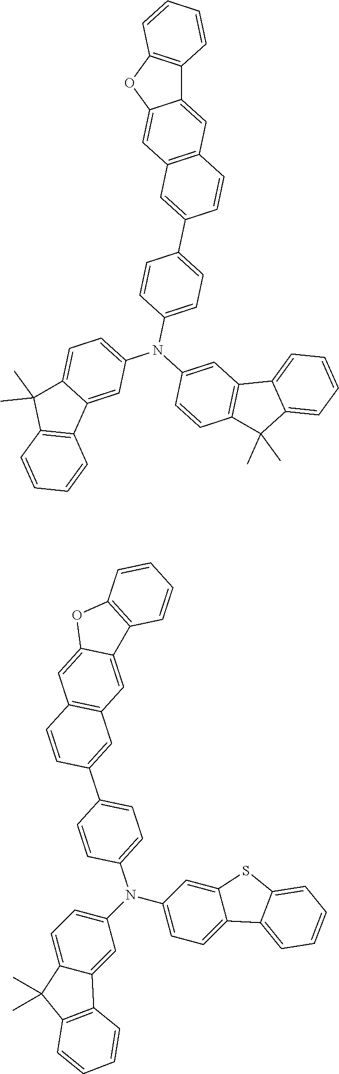 [SMILES: CC1(C)c2ccccc2-c2cc(N(c3ccc(-c4ccc5cc6c(cc5c4)oc4ccccc46)cc3)c3ccc4c(c3)-c3ccccc3C4(C)C)ccc21 ; CC1(C)c2ccccc2-c2cc(N(c3ccc(-c4ccc5cc6oc7ccccc7c6cc5c4)cc3)c3ccc4c(c3)sc3ccccc34)ccc21] 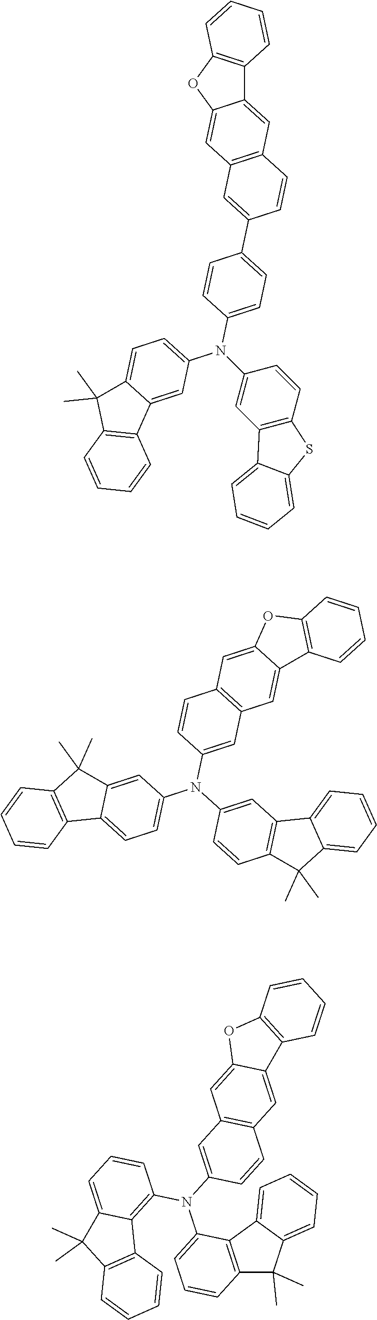 [SMILES: CC1(C)c2ccccc2-c2cc(N(c3ccc(-c4ccc5cc6c(cc5c4)oc4ccccc46)cc3)c3ccc4sc5ccccc5c4c3)ccc21 ; CC1(C)c2ccccc2-c2cc(N(c3ccc4c(c3)C(C)(C)c3ccccc3-4)c3ccc4cc5oc6ccccc6c5cc4c3)ccc21 ; CC1(C)c2ccccc2-c2c(N(c3ccc4cc5c(cc4c3)oc3ccccc35)c3cccc4c3-c3ccccc3C4(C)C)cccc21] 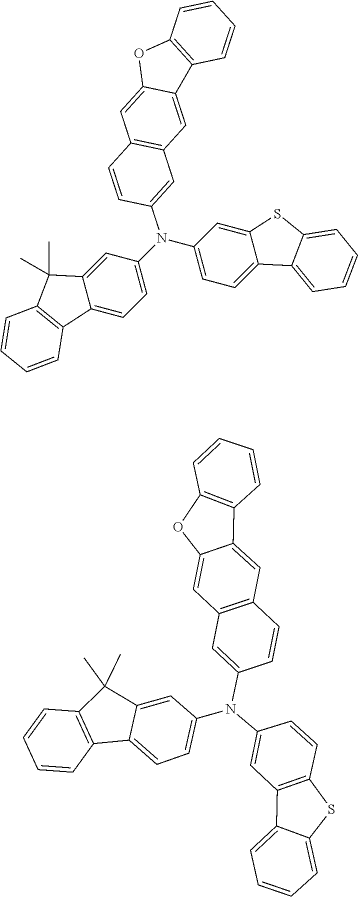 [SMILES: CC1(C)c2ccccc2-c2ccc(N(c3ccc4cc5oc6ccccc6c5cc4c3)c3ccc4c(c3)sc3ccccc34)cc21 ; CC1(C)c2ccccc2-c2ccc(N(c3ccc4cc5c(cc4c3)oc3ccccc35)c3ccc4sc5ccccc5c4c3)cc21] 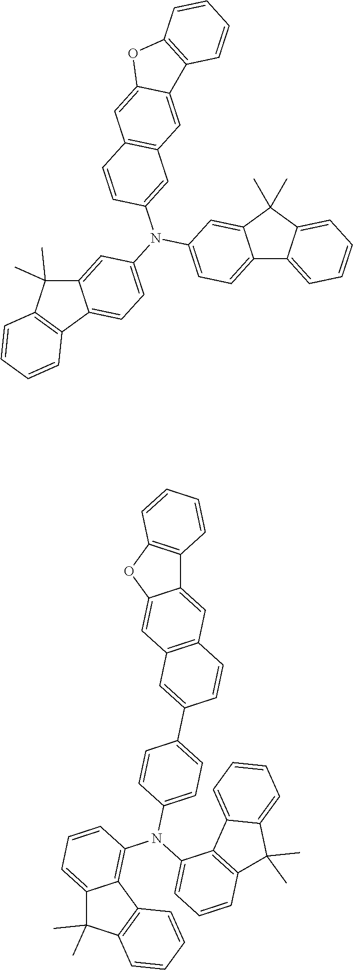 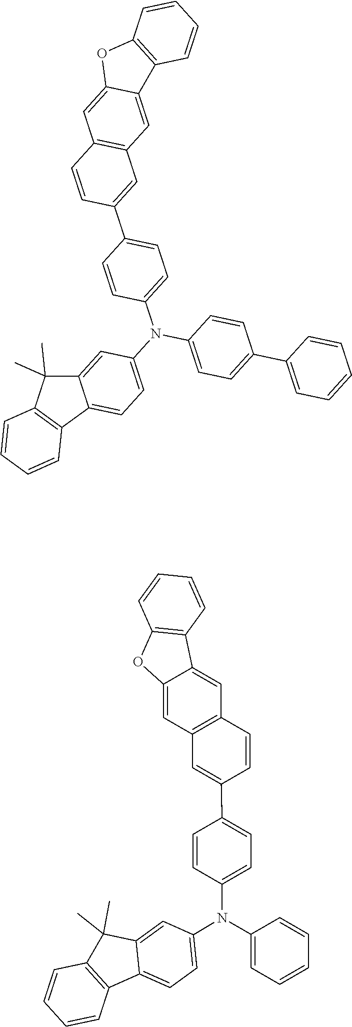 [SMILES: CC1(C)c2ccccc2-c2ccc(N(c3ccc(-c4ccccc4)cc3)c3ccc(-c4ccc5cc6oc7ccccc7c6cc5c4)cc3)cc21 ; CC1(C)c2ccccc2-c2ccc(N(c3ccccc3)c3ccc(-c4ccc5cc6c(cc5c4)oc4ccccc46)cc3)cc21] 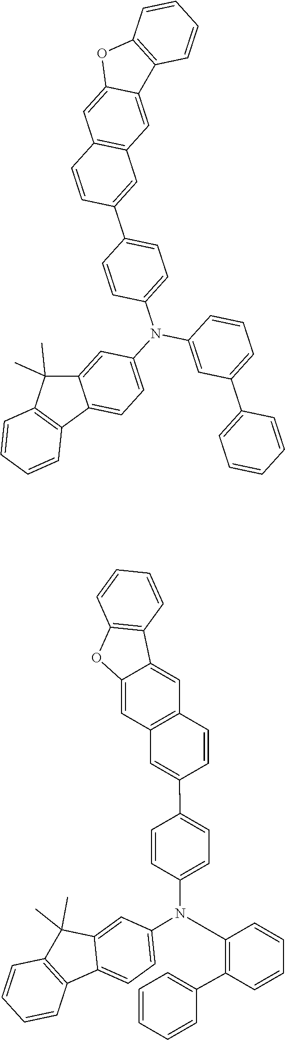 [SMILES: CC1(C)c2ccccc2-c2ccc(N(c3ccc(-c4ccc5cc6oc7ccccc7c6cc5c4)cc3)c3cccc(-c4ccccc4)c3)cc21 ; CC1(C)c2ccccc2-c2ccc(N(c3ccc(-c4ccc5cc6c(cc5c4)oc4ccccc46)cc3)c3ccccc3-c3ccccc3)cc21] 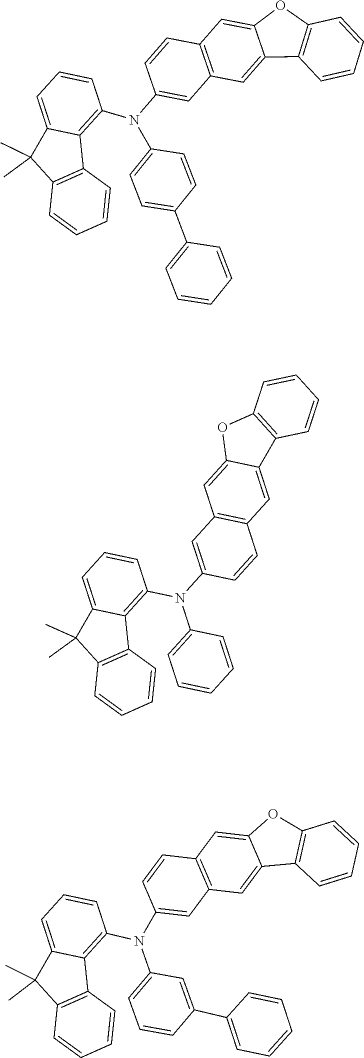 [SMILES: CC1(C)c2ccccc2-c2c(N(c3ccc(-c4ccccc4)cc3)c3ccc4cc5oc6ccccc6c5cc4c3)cccc21 ; CC1(C)c2ccccc2-c2c(N(c3ccccc3)c3ccc4cc5c(cc4c3)oc3ccccc35)cccc21 ; CC1(C)c2ccccc2-c2c(N(c3cccc(-c4ccccc4)c3)c3ccc4cc5oc6ccccc6c5cc4c3)cccc21] 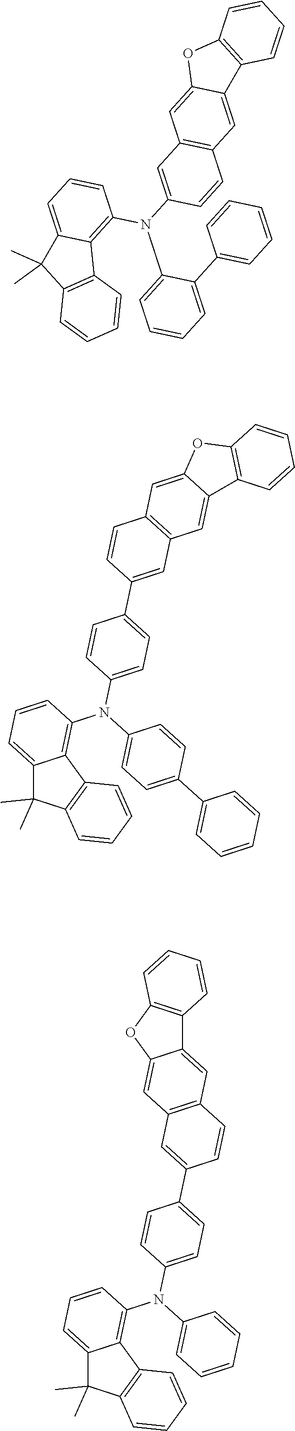 [SMILES: CC1(C)c2ccccc2-c2c(N(c3ccc4cc5c(cc4c3)oc3ccccc35)c3ccccc3-c3ccccc3)cccc21 ; CC1(C)c2ccccc2-c2c(N(c3ccc(-c4ccccc4)cc3)c3ccc(-c4ccc5cc6oc7ccccc7c6cc5c4)cc3)cccc21 ; CC1(C)c2ccccc2-c2c(N(c3ccccc3)c3ccc(-c4ccc5cc6c(cc5c4)oc4ccccc46)cc3)cccc21] 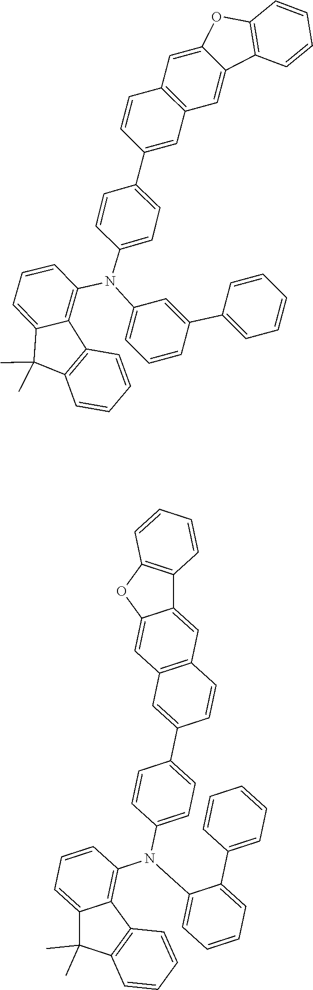 [SMILES: CC1(C)c2ccccc2-c2c(N(c3ccc(-c4ccc5cc6oc7ccccc7c6cc5c4)cc3)c3cccc(-c4ccccc4)c3)cccc21 ; CC1(C)c2ccccc2-c2c(N(c3ccc(-c4ccc5cc6c(cc5c4)oc4ccccc46)cc3)c3ccccc3-c3ccccc3)cccc21] 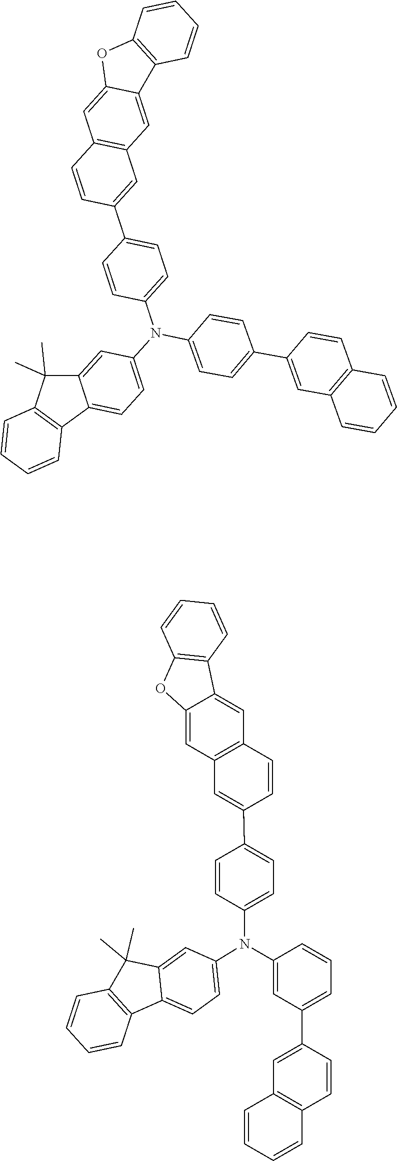 [SMILES: CC1(C)c2ccccc2-c2ccc(N(c3ccc(-c4ccc5ccccc5c4)cc3)c3ccc(-c4ccc5cc6oc7ccccc7c6cc5c4)cc3)cc21 ; CC1(C)c2ccccc2-c2ccc(N(c3ccc(-c4ccc5cc6c(cc5c4)oc4ccccc46)cc3)c3cccc(-c4ccc5ccccc5c4)c3)cc21] 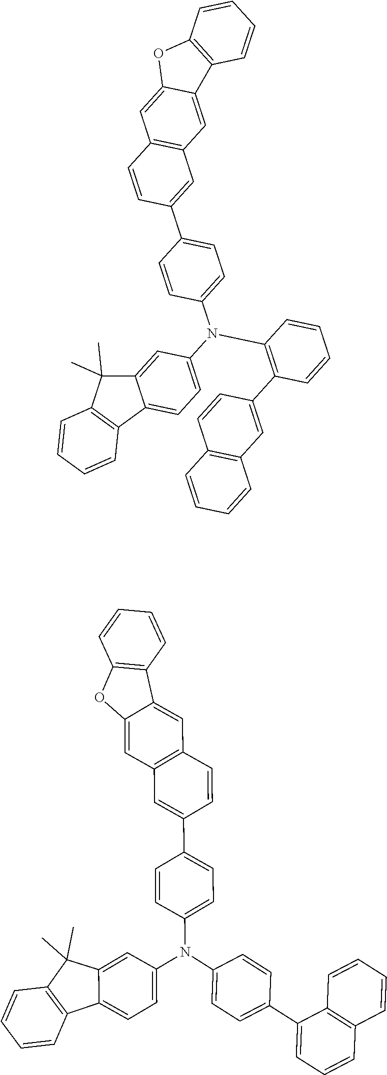 [SMILES: CC1(C)c2ccccc2-c2ccc(N(c3ccc(-c4ccc5cc6oc7ccccc7c6cc5c4)cc3)c3ccccc3-c3ccc4ccccc4c3)cc21 ; CC1(C)c2ccccc2-c2ccc(N(c3ccc(-c4ccc5cc6c(cc5c4)oc4ccccc46)cc3)c3ccc(-c4cccc5ccccc45)cc3)cc21] 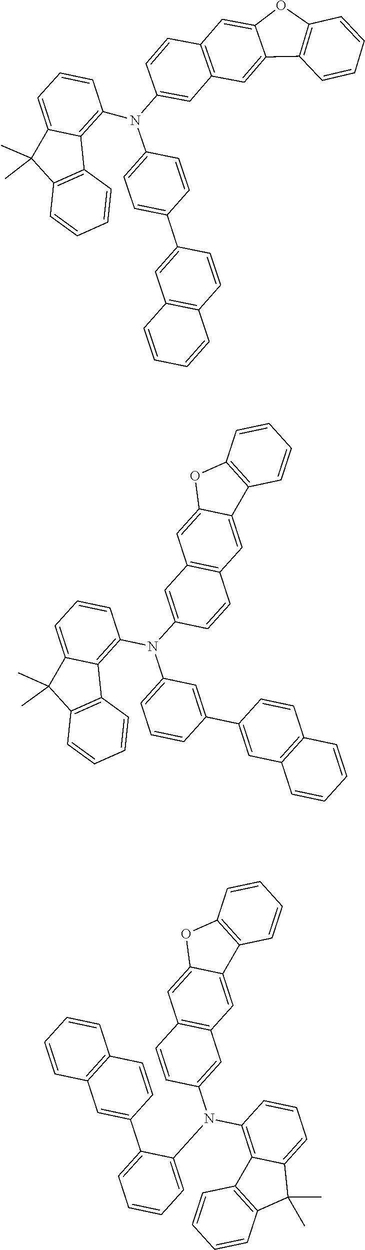 [SMILES: CC1(C)c2ccccc2-c2c(N(c3ccc(-c4ccc5ccccc5c4)cc3)c3ccc4cc5oc6ccccc6c5cc4c3)cccc21 ; CC1(C)c2ccccc2-c2c(N(c3cccc(-c4ccc5ccccc5c4)c3)c3ccc4cc5c(cc4c3)oc3ccccc35)cccc21 ; CC1(C)c2ccccc2-c2c(N(c3ccc4cc5oc6ccccc6c5cc4c3)c3ccccc3-c3ccc4ccccc4c3)cccc21] 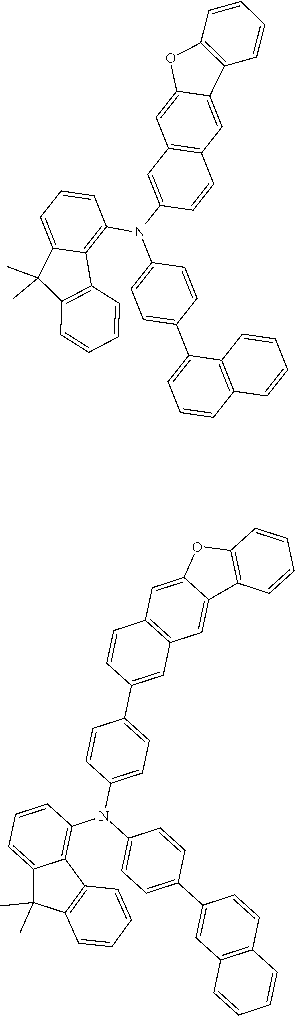 [SMILES: CC1(C)c2ccccc2-c2c(N(c3ccc(-c4cccc5ccccc45)cc3)c3ccc4cc5c(cc4c3)oc3ccccc35)cccc21 ; CC1(C)c2ccccc2-c2c(N(c3ccc(-c4ccc5ccccc5c4)cc3)c3ccc(-c4ccc5cc6oc7ccccc7c6cc5c4)cc3)cccc21] 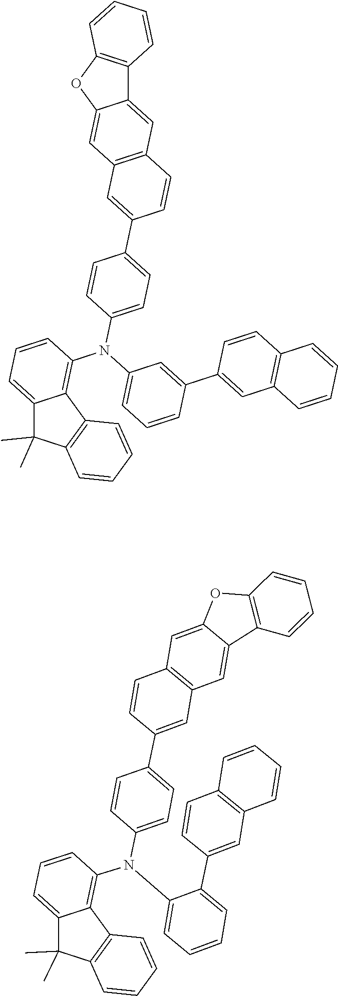 [SMILES: CC1(C)c2ccccc2-c2c(N(c3ccc(-c4ccc5cc6c(cc5c4)oc4ccccc46)cc3)c3cccc(-c4ccc5ccccc5c4)c3)cccc21 ; CC1(C)c2ccccc2-c2c(N(c3ccc(-c4ccc5cc6oc7ccccc7c6cc5c4)cc3)c3ccccc3-c3ccc4ccccc4c3)cccc21] 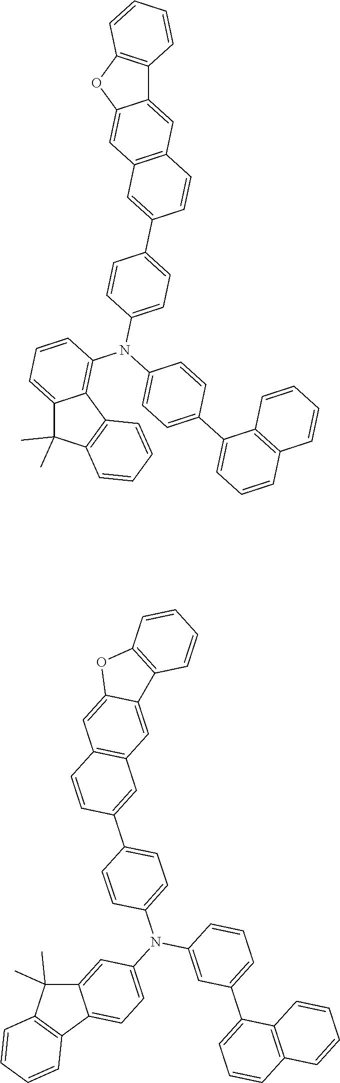 [SMILES: CC1(C)c2ccccc2-c2c(N(c3ccc(-c4ccc5cc6c(cc5c4)oc4ccccc46)cc3)c3ccc(-c4cccc5ccccc45)cc3)cccc21 ; CC1(C)c2ccccc2-c2ccc(N(c3ccc(-c4ccc5cc6oc7ccccc7c6cc5c4)cc3)c3cccc(-c4cccc5ccccc45)c3)cc21] 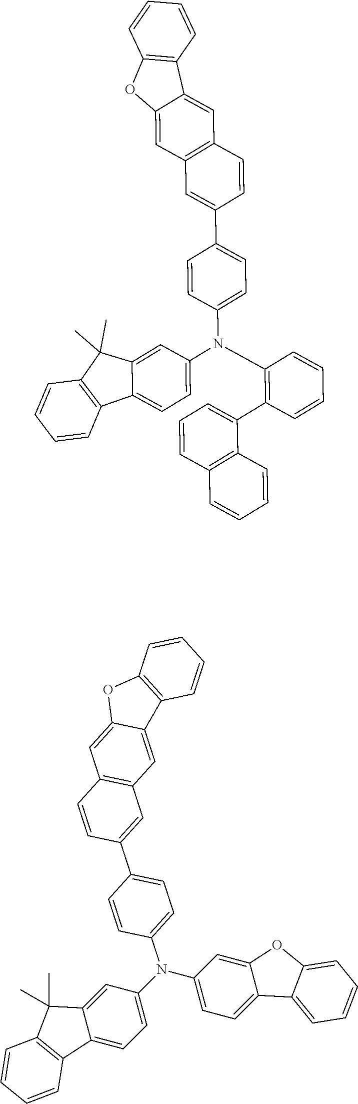 [SMILES: CC1(C)c2ccccc2-c2ccc(N(c3ccc(-c4ccc5cc6c(cc5c4)oc4ccccc46)cc3)c3ccccc3-c3cccc4ccccc34)cc21 ; CC1(C)c2ccccc2-c2ccc(N(c3ccc(-c4ccc5cc6oc7ccccc7c6cc5c4)cc3)c3ccc4c(c3)oc3ccccc34)cc21] 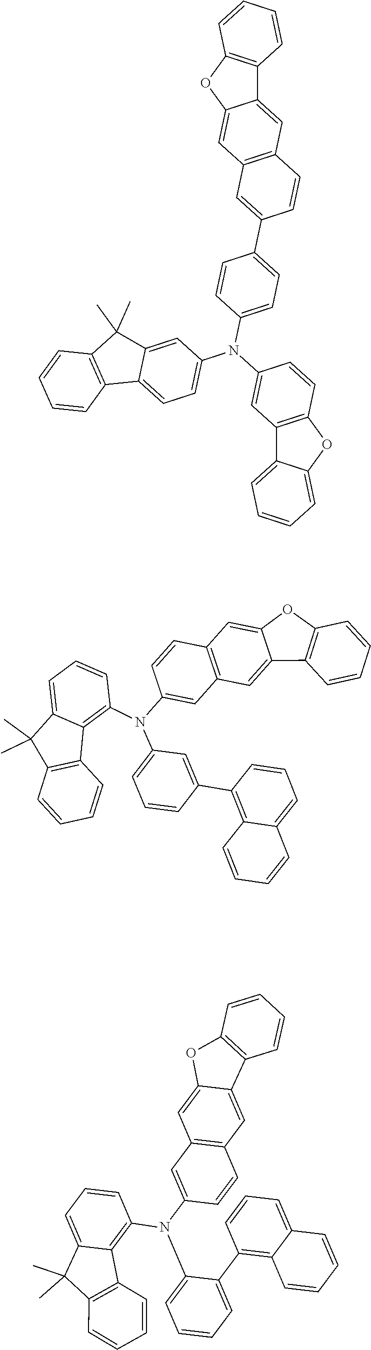 [SMILES: CC1(C)c2ccccc2-c2ccc(N(c3ccc(-c4ccc5cc6c(cc5c4)oc4ccccc46)cc3)c3ccc4oc5ccccc5c4c3)cc21 ; CC1(C)c2ccccc2-c2c(N(c3cccc(-c4cccc5ccccc45)c3)c3ccc4cc5oc6ccccc6c5cc4c3)cccc21 ; CC1(C)c2ccccc2-c2c(N(c3ccc4cc5c(cc4c3)oc3ccccc35)c3ccccc3-c3cccc4ccccc34)cccc21] 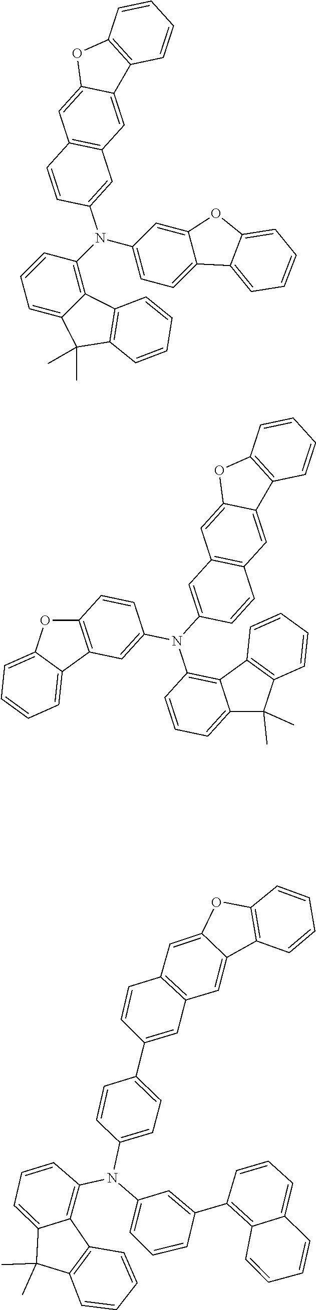 [SMILES: CC1(C)c2ccccc2-c2c(N(c3ccc4cc5oc6ccccc6c5cc4c3)c3ccc4c(c3)oc3ccccc34)cccc21 ; CC1(C)c2ccccc2-c2c(N(c3ccc4cc5c(cc4c3)oc3ccccc35)c3ccc4oc5ccccc5c4c3)cccc21 ; CC1(C)c2ccccc2-c2c(N(c3ccc(-c4ccc5cc6oc7ccccc7c6cc5c4)cc3)c3cccc(-c4cccc5ccccc45)c3)cccc21] 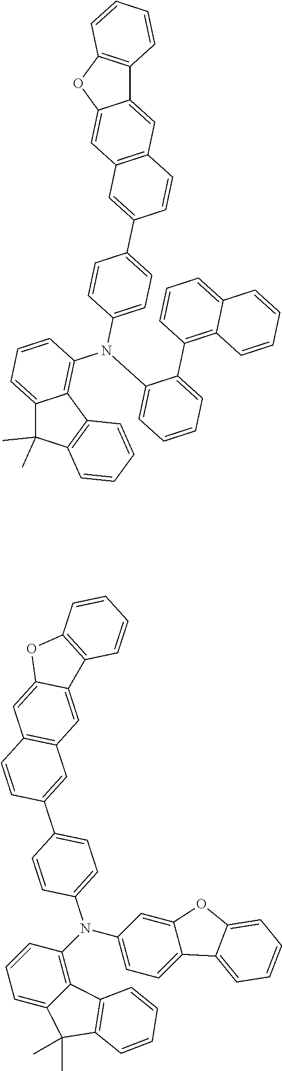 [SMILES: CC1(C)c2ccccc2-c2c(N(c3ccc(-c4ccc5cc6c(cc5c4)oc4ccccc46)cc3)c3ccccc3-c3cccc4ccccc34)cccc21 ; CC1(C)c2ccccc2-c2c(N(c3ccc(-c4ccc5cc6oc7ccccc7c6cc5c4)cc3)c3ccc4c(c3)oc3ccccc34)cccc21] 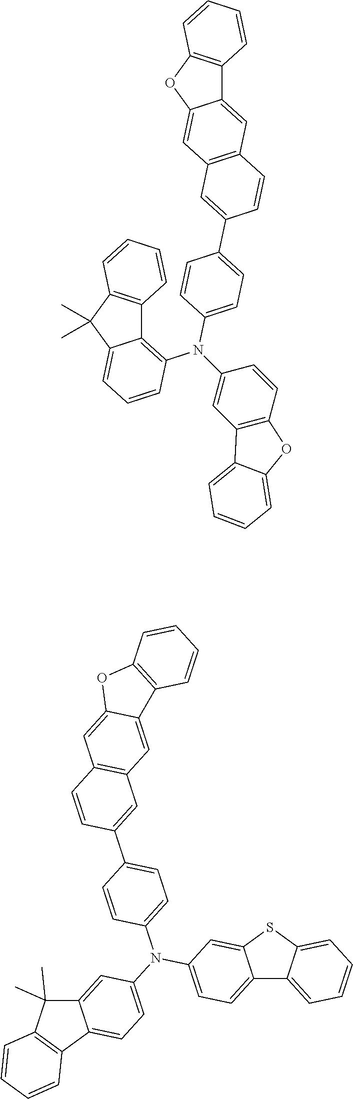 [SMILES: CC1(C)c2ccccc2-c2c(N(c3ccc(-c4ccc5cc6c(cc5c4)oc4ccccc46)cc3)c3ccc4oc5ccccc5c4c3)cccc21 ; CC1(C)c2ccccc2-c2ccc(N(c3ccc(-c4ccc5cc6oc7ccccc7c6cc5c4)cc3)c3ccc4c(c3)sc3ccccc34)cc21] 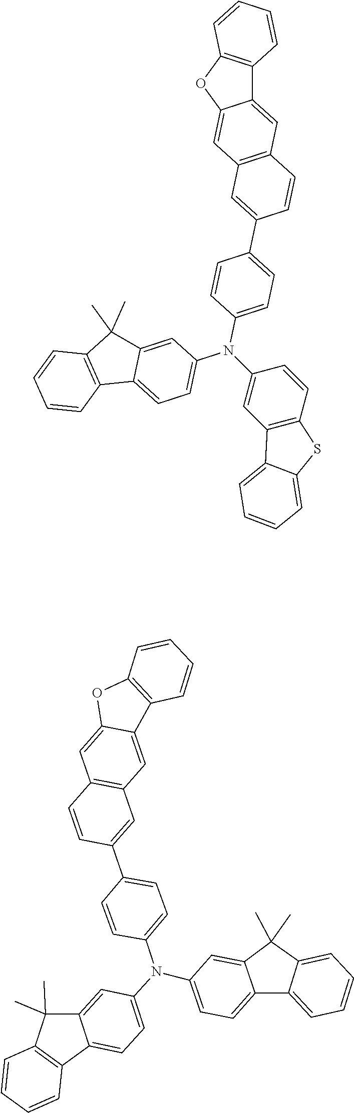 [SMILES: CC1(C)c2ccccc2-c2ccc(N(c3ccc(-c4ccc5cc6c(cc5c4)oc4ccccc46)cc3)c3ccc4sc5ccccc5c4c3)cc21 ; CC1(C)c2ccccc2-c2ccc(N(c3ccc(-c4ccc5cc6oc7ccccc7c6cc5c4)cc3)c3ccc4c(c3)C(C)(C)c3ccccc3-4)cc21] 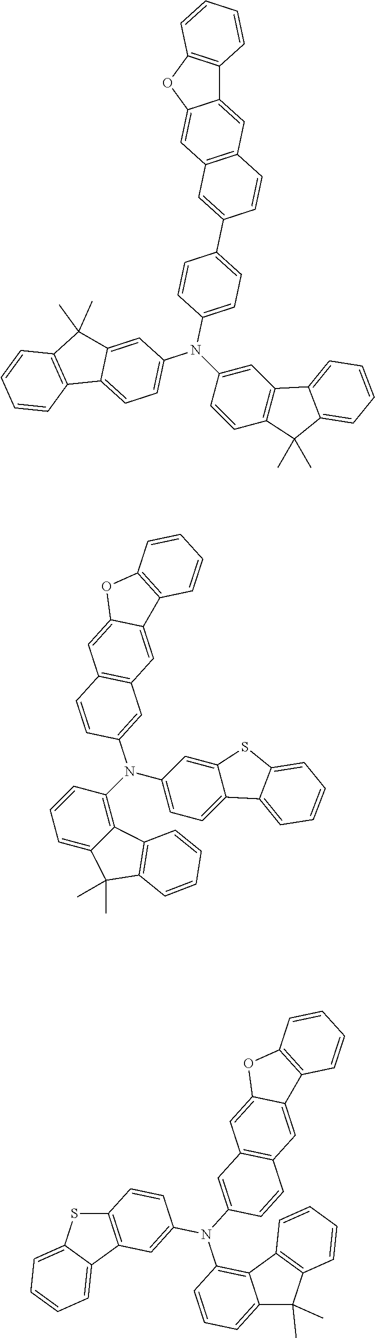 [SMILES: CC1(C)c2ccccc2-c2cc(N(c3ccc(-c4ccc5cc6c(cc5c4)oc4ccccc46)cc3)c3ccc4c(c3)C(C)(C)c3ccccc3-4)ccc21 ; CC1(C)c2ccccc2-c2c(N(c3ccc4cc5oc6ccccc6c5cc4c3)c3ccc4c(c3)sc3ccccc34)cccc21 ; CC1(C)c2ccccc2-c2c(N(c3ccc4cc5c(cc4c3)oc3ccccc35)c3ccc4sc5ccccc5c4c3)cccc21] 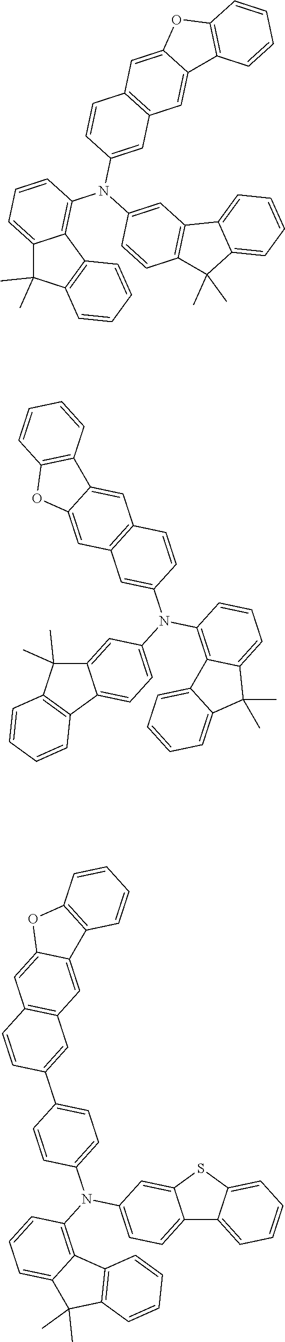 [SMILES: CC1(C)c2ccccc2-c2cc(N(c3ccc4cc5oc6ccccc6c5cc4c3)c3cccc4c3-c3ccccc3C4(C)C)ccc21 ; CC1(C)c2ccccc2-c2ccc(N(c3ccc4cc5c(cc4c3)oc3ccccc35)c3cccc4c3-c3ccccc3C4(C)C)cc21 ; CC1(C)c2ccccc2-c2c(N(c3ccc(-c4ccc5cc6oc7ccccc7c6cc5c4)cc3)c3ccc4c(c3)sc3ccccc34)cccc21] 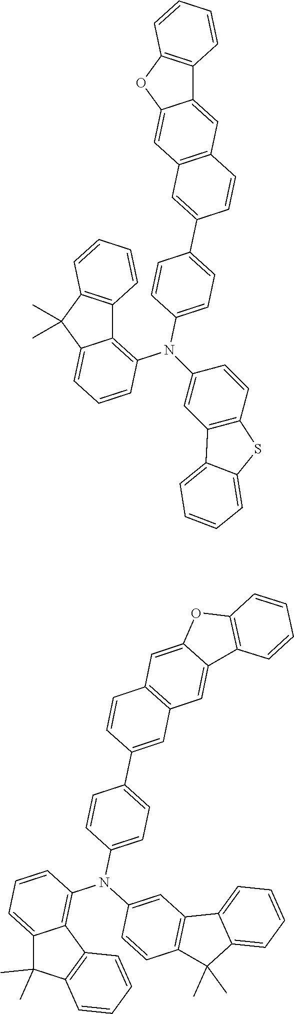 [SMILES: CC1(C)c2ccccc2-c2c(N(c3ccc(-c4ccc5cc6c(cc5c4)oc4ccccc46)cc3)c3ccc4sc5ccccc5c4c3)cccc21 ; CC1(C)c2ccccc2-c2cc(N(c3ccc(-c4ccc5cc6oc7ccccc7c6cc5c4)cc3)c3cccc4c3-c3ccccc3C4(C)C)ccc21] 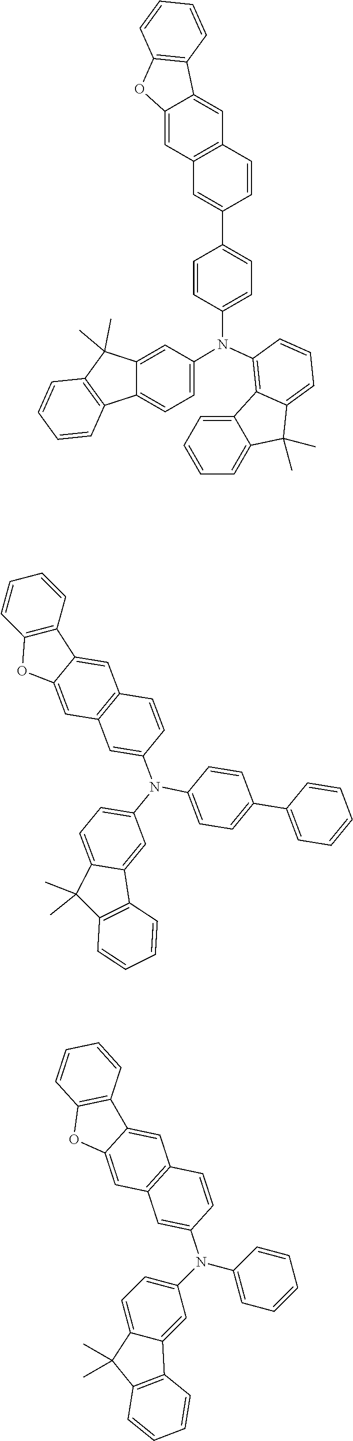 [SMILES: CC1(C)c2ccccc2-c2ccc(N(c3ccc(-c4ccc5cc6c(cc5c4)oc4ccccc46)cc3)c3cccc4c3-c3ccccc3C4(C)C)cc21 ; CC1(C)c2ccccc2-c2cc(N(c3ccc(-c4ccccc4)cc3)c3ccc4cc5c(cc4c3)oc3ccccc35)ccc21 ; CC1(C)c2ccccc2-c2cc(N(c3ccccc3)c3ccc4cc5c(cc4c3)oc3ccccc35)ccc21] 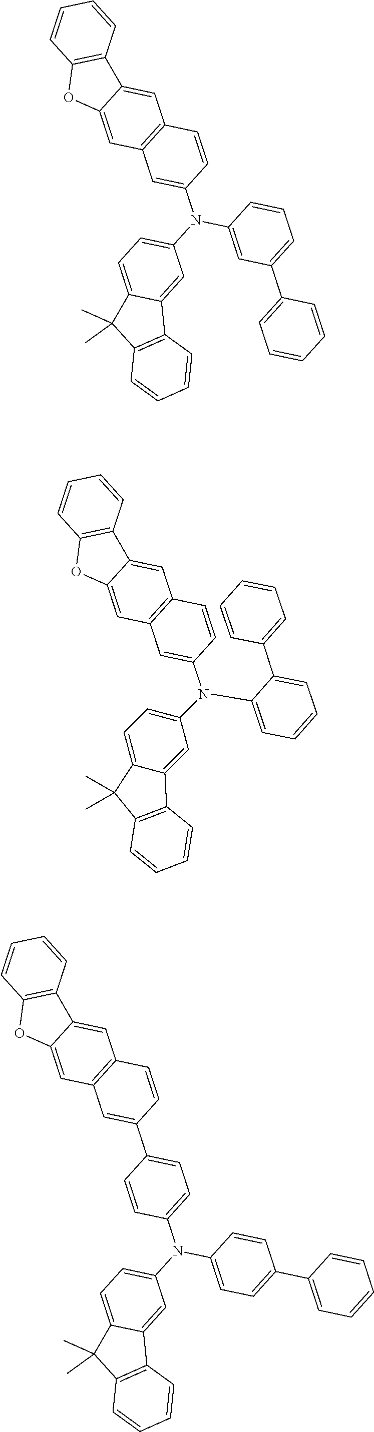 [SMILES: CC1(C)c2ccccc2-c2cc(N(c3cccc(-c4ccccc4)c3)c3ccc4cc5c(cc4c3)oc3ccccc35)ccc21 ; CC1(C)c2ccccc2-c2cc(N(c3ccc4cc5c(cc4c3)oc3ccccc35)c3ccccc3-c3ccccc3)ccc21 ; CC1(C)c2ccccc2-c2cc(N(c3ccc(-c4ccccc4)cc3)c3ccc(-c4ccc5cc6c(cc5c4)oc4ccccc46)cc3)ccc21] 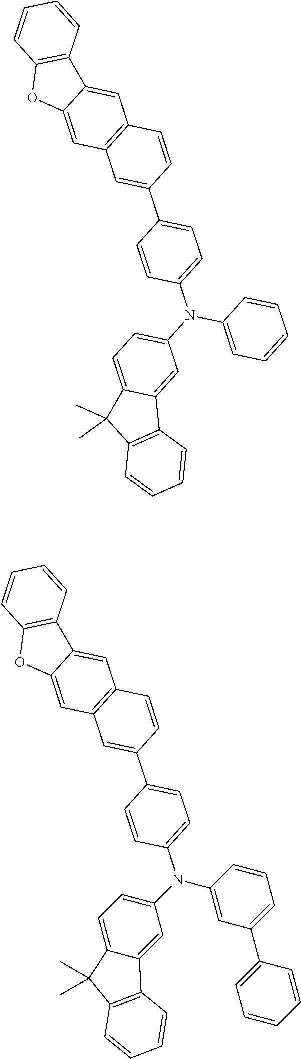 [SMILES: CC1(C)c2ccccc2-c2cc(N(c3ccccc3)c3ccc(-c4ccc5cc6c(cc5c4)oc4ccccc46)cc3)ccc21 ; CC1(C)c2ccccc2-c2cc(N(c3ccc(-c4ccc5cc6c(cc5c4)oc4ccccc46)cc3)c3cccc(-c4ccccc4)c3)ccc21] 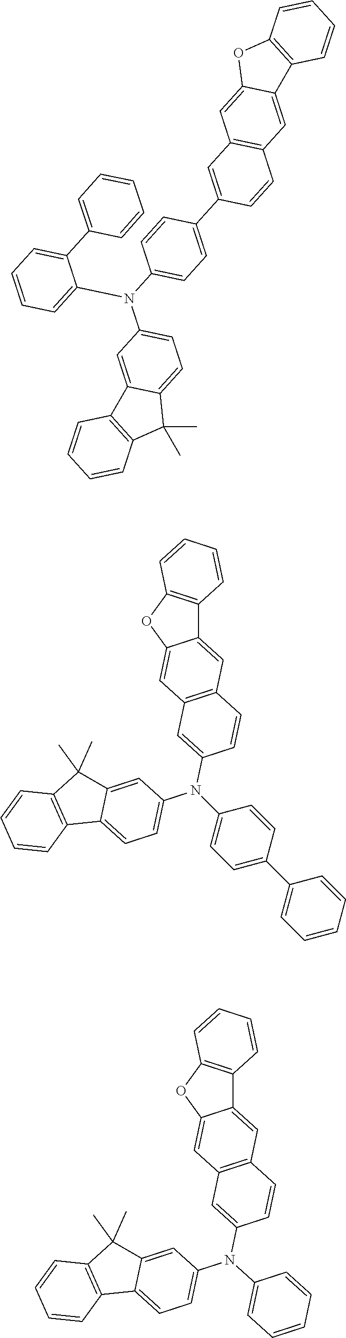 [SMILES: CC1(C)c2ccccc2-c2cc(N(c3ccc(-c4ccc5cc6c(cc5c4)oc4ccccc46)cc3)c3ccccc3-c3ccccc3)ccc21 ; CC1(C)c2ccccc2-c2ccc(N(c3ccc(-c4ccccc4)cc3)c3ccc4cc5c(cc4c3)oc3ccccc35)cc21 ; CC1(C)c2ccccc2-c2ccc(N(c3ccccc3)c3ccc4cc5c(cc4c3)oc3ccccc35)cc21] 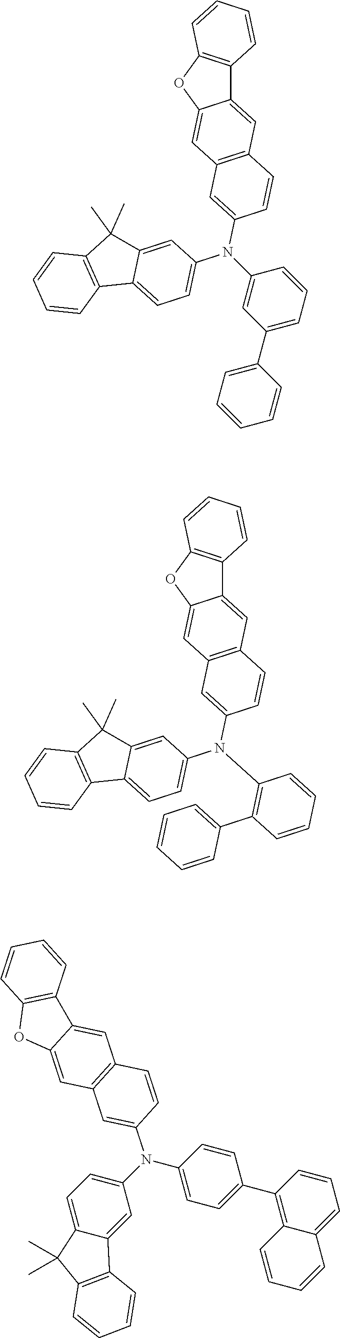 [SMILES: CC1(C)c2ccccc2-c2ccc(N(c3cccc(-c4ccccc4)c3)c3ccc4cc5c(cc4c3)oc3ccccc35)cc21 ; CC1(C)c2ccccc2-c2ccc(N(c3ccc4cc5c(cc4c3)oc3ccccc35)c3ccccc3-c3ccccc3)cc21 ; CC1(C)c2ccccc2-c2cc(N(c3ccc(-c4cccc5ccccc45)cc3)c3ccc4cc5c(cc4c3)oc3ccccc35)ccc21] 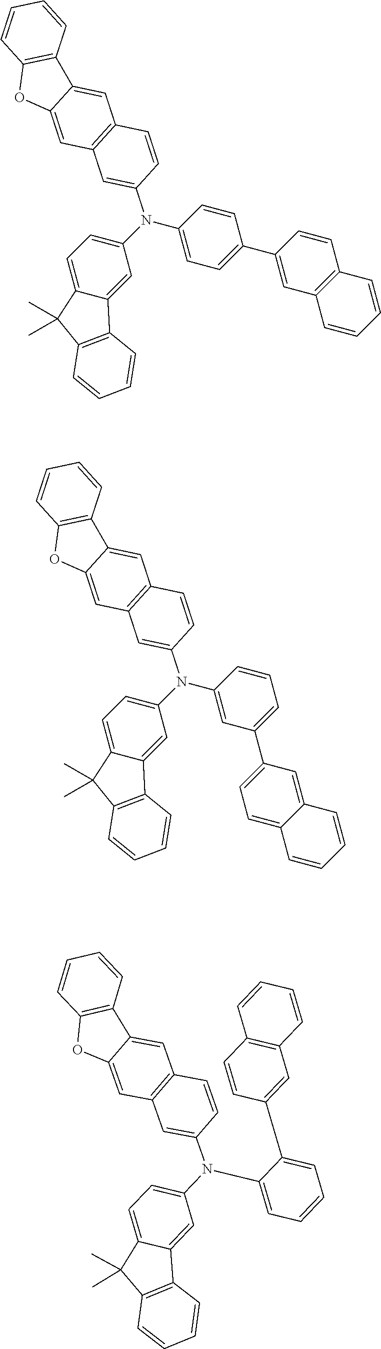 [SMILES: CC1(C)c2ccccc2-c2cc(N(c3ccc(-c4ccc5ccccc5c4)cc3)c3ccc4cc5c(cc4c3)oc3ccccc35)ccc21 ; CC1(C)c2ccccc2-c2cc(N(c3cccc(-c4ccc5ccccc5c4)c3)c3ccc4cc5c(cc4c3)oc3ccccc35)ccc21 ; CC1(C)c2ccccc2-c2cc(N(c3ccc4cc5c(cc4c3)oc3ccccc35)c3ccccc3-c3ccc4ccccc4c3)ccc21] 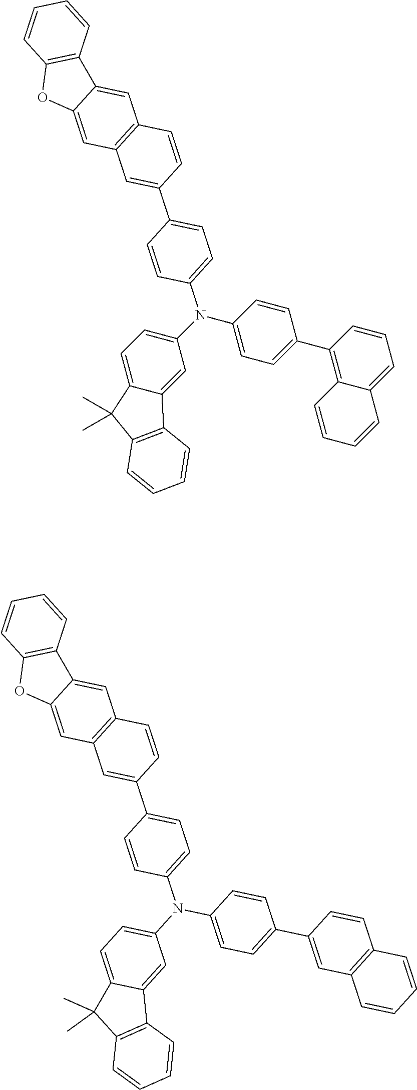 [SMILES: CC1(C)c2ccccc2-c2cc(N(c3ccc(-c4ccc5cc6c(cc5c4)oc4ccccc46)cc3)c3ccc(-c4cccc5ccccc45)cc3)ccc21 ; CC1(C)c2ccccc2-c2cc(N(c3ccc(-c4ccc5ccccc5c4)cc3)c3ccc(-c4ccc5cc6c(cc5c4)oc4ccccc46)cc3)ccc21] 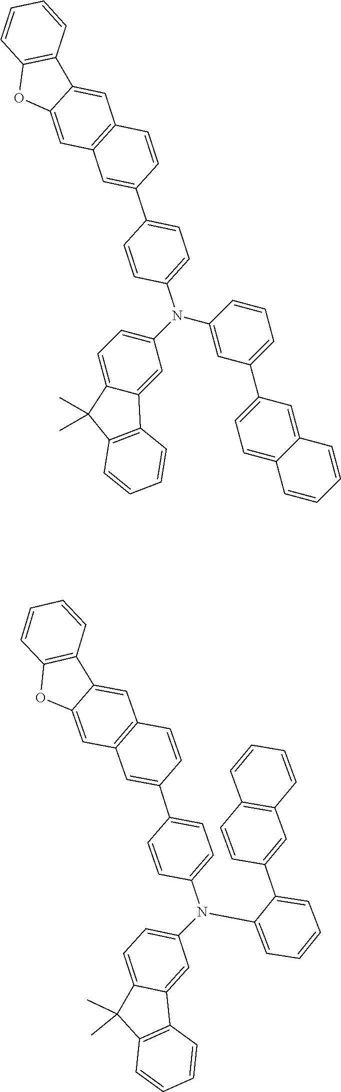 [SMILES: CC1(C)c2ccccc2-c2cc(N(c3ccc(-c4ccc5cc6c(cc5c4)oc4ccccc46)cc3)c3cccc(-c4ccc5ccccc5c4)c3)ccc21 ; CC1(C)c2ccccc2-c2cc(N(c3ccc(-c4ccc5cc6c(cc5c4)oc4ccccc46)cc3)c3ccccc3-c3ccc4ccccc4c3)ccc21] 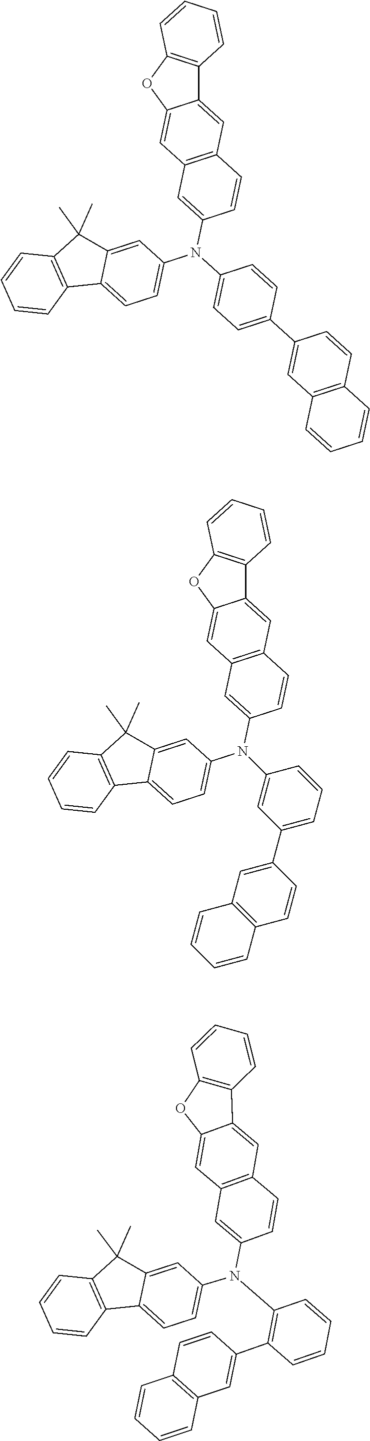 [SMILES: CC1(C)c2ccccc2-c2ccc(N(c3ccc(-c4ccc5ccccc5c4)cc3)c3ccc4cc5c(cc4c3)oc3ccccc35)cc21 ; CC1(C)c2ccccc2-c2ccc(N(c3cccc(-c4ccc5ccccc5c4)c3)c3ccc4cc5c(cc4c3)oc3ccccc35)cc21 ; CC1(C)c2ccccc2-c2ccc(N(c3ccc4cc5c(cc4c3)oc3ccccc35)c3ccccc3-c3ccc4ccccc4c3)cc21] 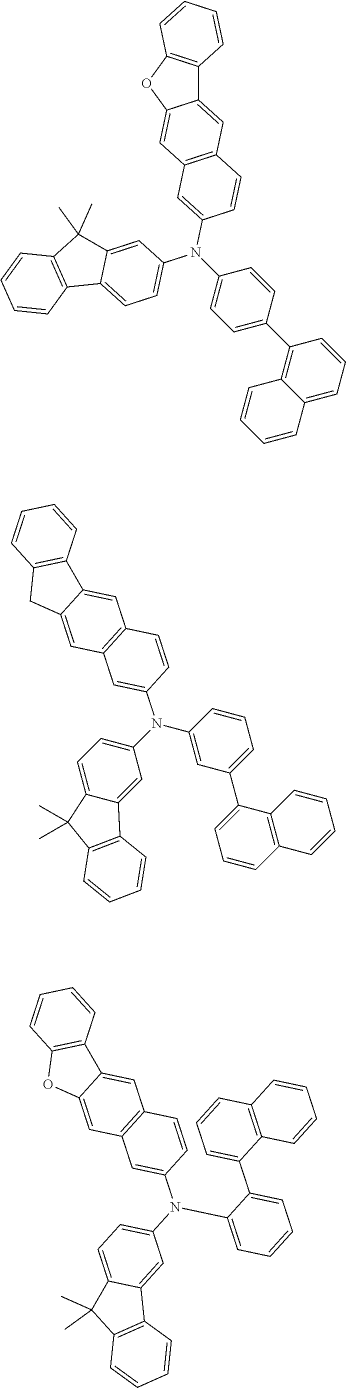 [SMILES: CC1(C)c2ccccc2-c2ccc(N(c3ccc(-c4cccc5ccccc45)cc3)c3ccc4cc5c(cc4c3)oc3ccccc35)cc21 ; CC1(C)c2ccccc2-c2cc(N(c3cccc(-c4cccc5ccccc45)c3)c3ccc4cc5c(cc4c3)Cc3ccccc3-5)ccc21 ; CC1(C)c2ccccc2-c2cc(N(c3ccc4cc5c(cc4c3)oc3ccccc35)c3ccccc3-c3cccc4ccccc34)ccc21] 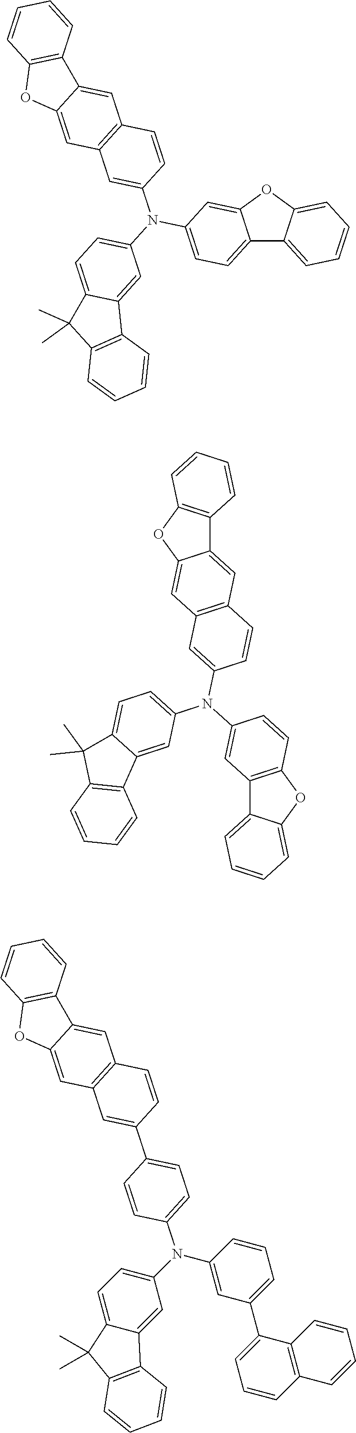 [SMILES: CC1(C)c2ccccc2-c2cc(N(c3ccc4cc5c(cc4c3)oc3ccccc35)c3ccc4c(c3)oc3ccccc34)ccc21 ; CC1(C)c2ccccc2-c2cc(N(c3ccc4cc5c(cc4c3)oc3ccccc35)c3ccc4oc5ccccc5c4c3)ccc21 ; CC1(C)c2ccccc2-c2cc(N(c3ccc(-c4ccc5cc6c(cc5c4)oc4ccccc46)cc3)c3cccc(-c4cccc5ccccc45)c3)ccc21] 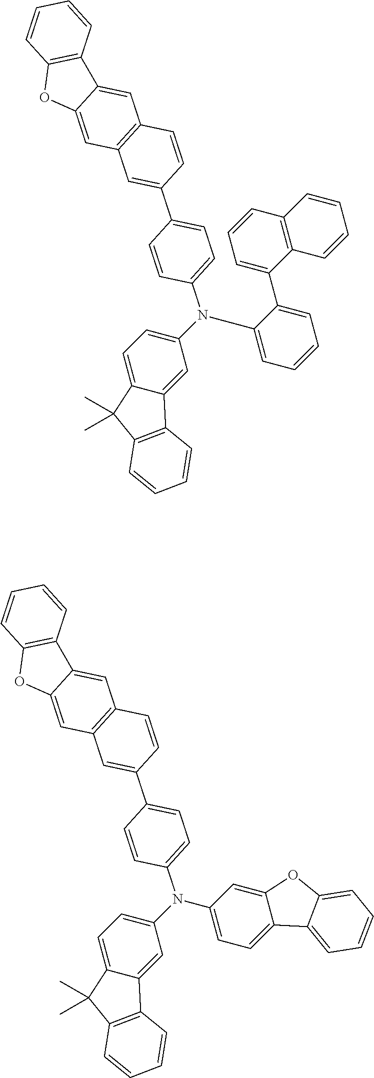 [SMILES: CC1(C)c2ccccc2-c2cc(N(c3ccc(-c4ccc5cc6c(cc5c4)oc4ccccc46)cc3)c3ccccc3-c3cccc4ccccc34)ccc21 ; CC1(C)c2ccccc2-c2cc(N(c3ccc(-c4ccc5cc6c(cc5c4)oc4ccccc46)cc3)c3ccc4c(c3)oc3ccccc34)ccc21] 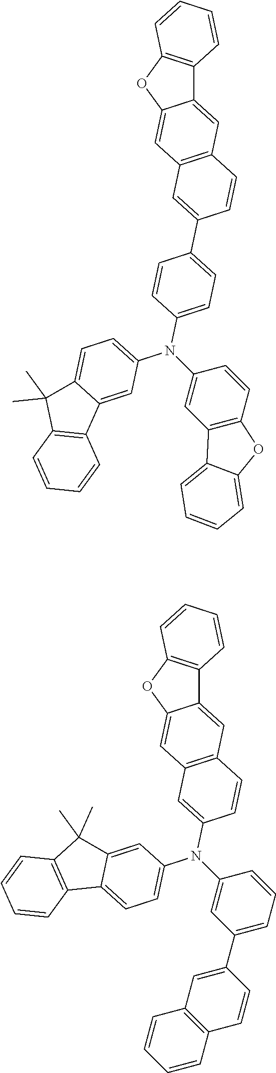 [SMILES: CC1(C)c2ccccc2-c2cc(N(c3ccc(-c4ccc5cc6c(cc5c4)oc4ccccc46)cc3)c3ccc4oc5ccccc5c4c3)ccc21 ; CC1(C)c2ccccc2-c2ccc(N(c3cccc(-c4ccc5ccccc5c4)c3)c3ccc4cc5c(cc4c3)oc3ccccc35)cc21] 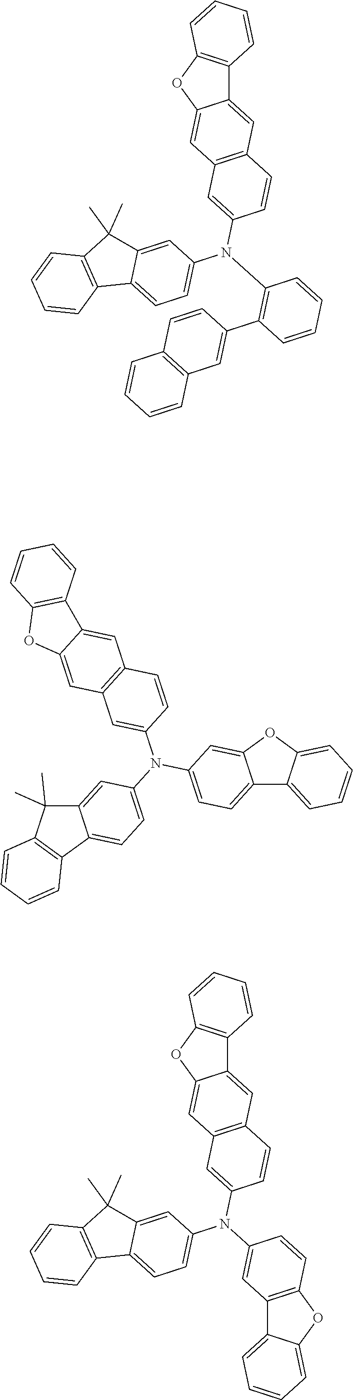 [SMILES: CC1(C)c2ccccc2-c2ccc(N(c3ccc4cc5c(cc4c3)oc3ccccc35)c3ccccc3-c3ccc4ccccc4c3)cc21 ; CC1(C)c2ccccc2-c2ccc(N(c3ccc4cc5c(cc4c3)oc3ccccc35)c3ccc4c(c3)oc3ccccc34)cc21 ; CC1(C)c2ccccc2-c2ccc(N(c3ccc4cc5c(cc4c3)oc3ccccc35)c3ccc4oc5ccccc5c4c3)cc21] 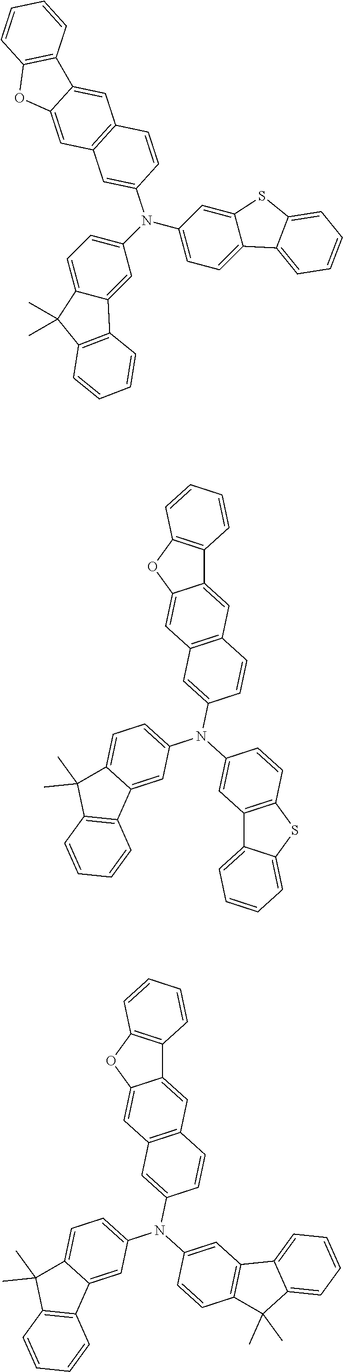 [SMILES: CC1(C)c2ccccc2-c2cc(N(c3ccc4cc5c(cc4c3)oc3ccccc35)c3ccc4c(c3)sc3ccccc34)ccc21 ; CC1(C)c2ccccc2-c2cc(N(c3ccc4cc5c(cc4c3)oc3ccccc35)c3ccc4sc5ccccc5c4c3)ccc21 ; CC1(C)c2ccccc2-c2cc(N(c3ccc4c(c3)-c3ccccc3C4(C)C)c3ccc4cc5c(cc4c3)oc3ccccc35)ccc21] 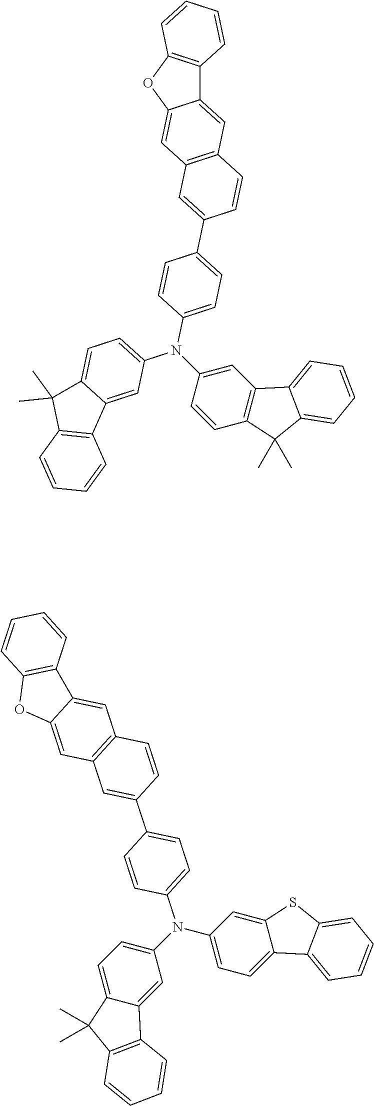 [SMILES: CC1(C)c2ccccc2-c2cc(N(c3ccc(-c4ccc5cc6c(cc5c4)oc4ccccc46)cc3)c3ccc4c(c3)-c3ccccc3C4(C)C)ccc21 ; CC1(C)c2ccccc2-c2cc(N(c3ccc(-c4ccc5cc6c(cc5c4)oc4ccccc46)cc3)c3ccc4c(c3)sc3ccccc34)ccc21] 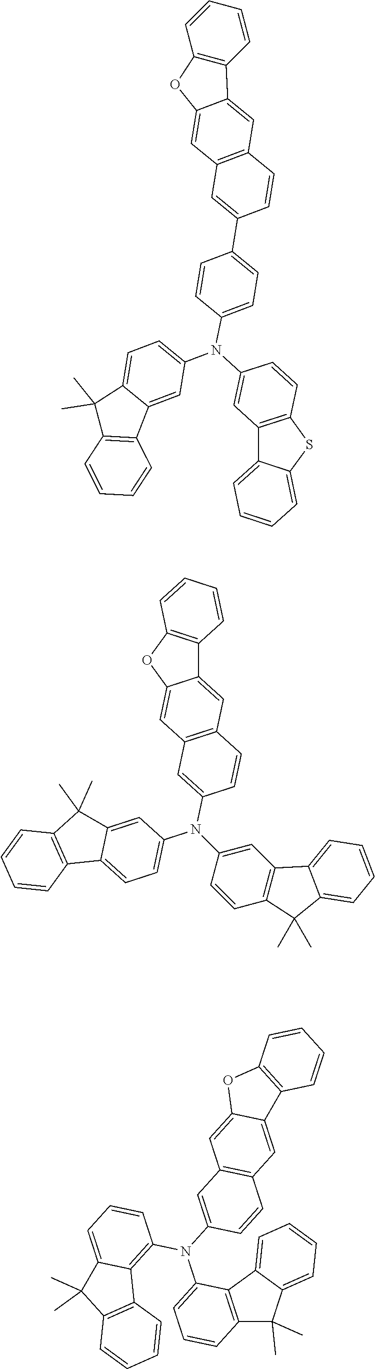 [SMILES: CC1(C)c2ccccc2-c2cc(N(c3ccc(-c4ccc5cc6c(cc5c4)oc4ccccc46)cc3)c3ccc4sc5ccccc5c4c3)ccc21 ; CC1(C)c2ccccc2-c2cc(N(c3ccc4c(c3)C(C)(C)c3ccccc3-4)c3ccc4cc5c(cc4c3)oc3ccccc35)ccc21 ; CC1(C)c2ccccc2-c2c(N(c3ccc4cc5c(cc4c3)oc3ccccc35)c3cccc4c3-c3ccccc3C4(C)C)cccc21] 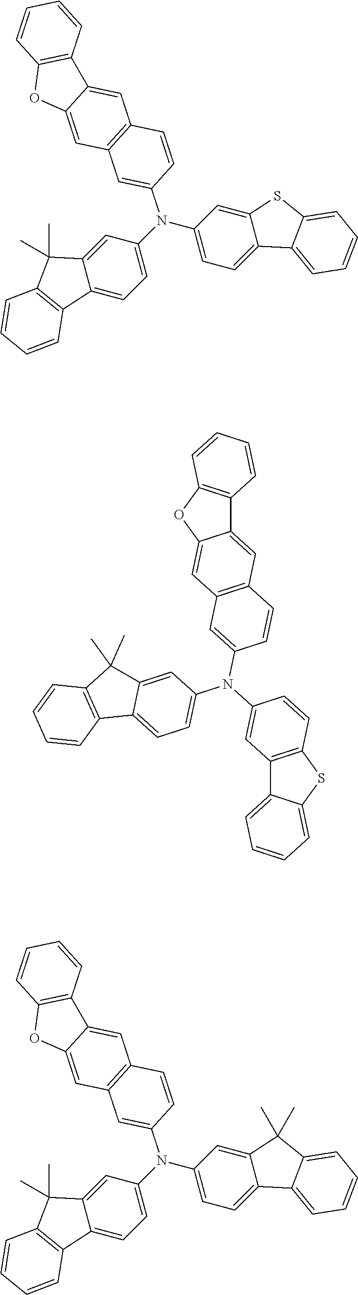 [SMILES: CC1(C)c2ccccc2-c2ccc(N(c3ccc4cc5c(cc4c3)oc3ccccc35)c3ccc4c(c3)sc3ccccc34)cc21 ; CC1(C)c2ccccc2-c2ccc(N(c3ccc4cc5c(cc4c3)oc3ccccc35)c3ccc4sc5ccccc5c4c3)cc21 ; CC1(C)c2ccccc2-c2ccc(N(c3ccc4c(c3)C(C)(C)c3ccccc3-4)c3ccc4cc5c(cc4c3)oc3ccccc35)cc21] 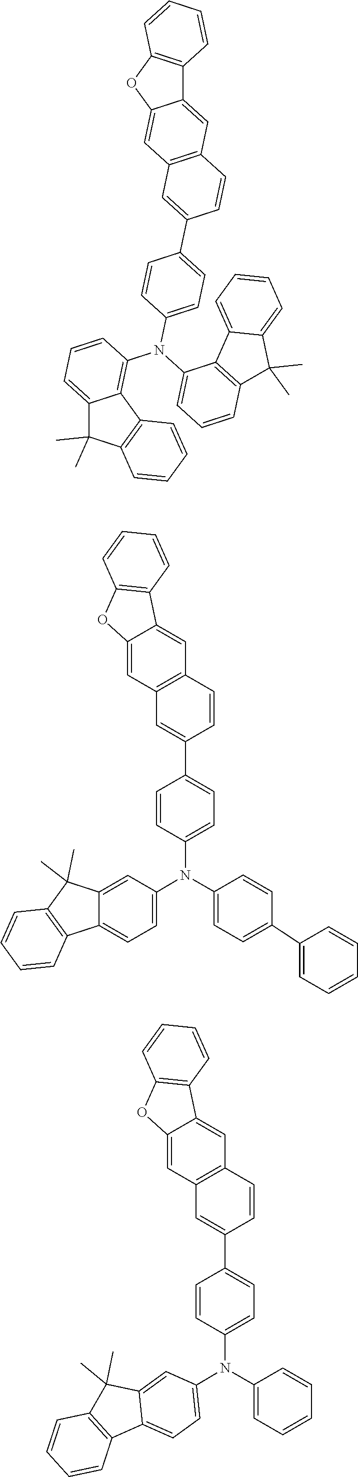 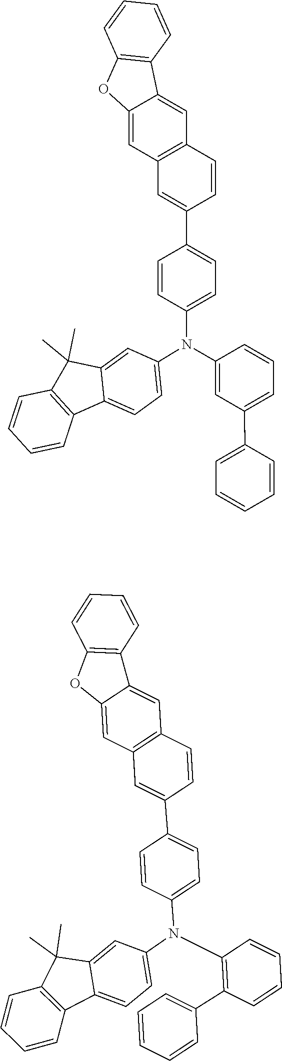 [SMILES: CC1(C)c2ccccc2-c2ccc(N(c3ccc(-c4ccc5cc6c(cc5c4)oc4ccccc46)cc3)c3cccc(-c4ccccc4)c3)cc21 ; CC1(C)c2ccccc2-c2ccc(N(c3ccc(-c4ccc5cc6c(cc5c4)oc4ccccc46)cc3)c3ccccc3-c3ccccc3)cc21] 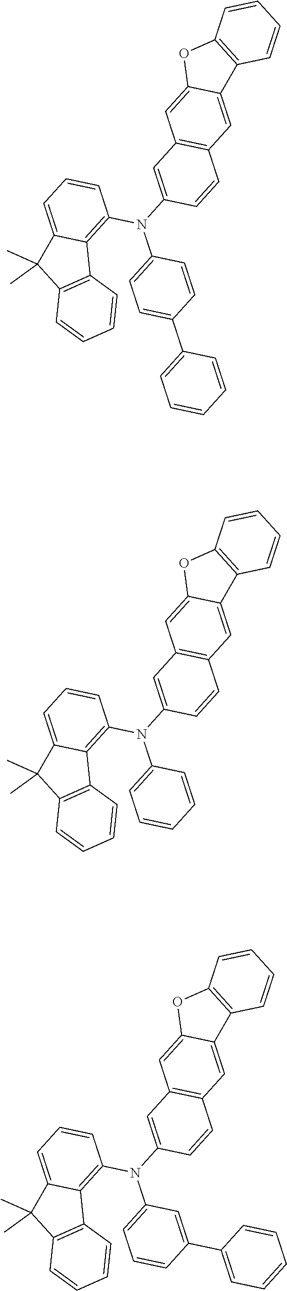 [SMILES: CC1(C)c2ccccc2-c2c(N(c3ccc(-c4ccccc4)cc3)c3ccc4cc5c(cc4c3)oc3ccccc35)cccc21 ; CC1(C)c2ccccc2-c2c(N(c3ccccc3)c3ccc4cc5c(cc4c3)oc3ccccc35)cccc21 ; CC1(C)c2ccccc2-c2c(N(c3cccc(-c4ccccc4)c3)c3ccc4cc5c(cc4c3)oc3ccccc35)cccc21] 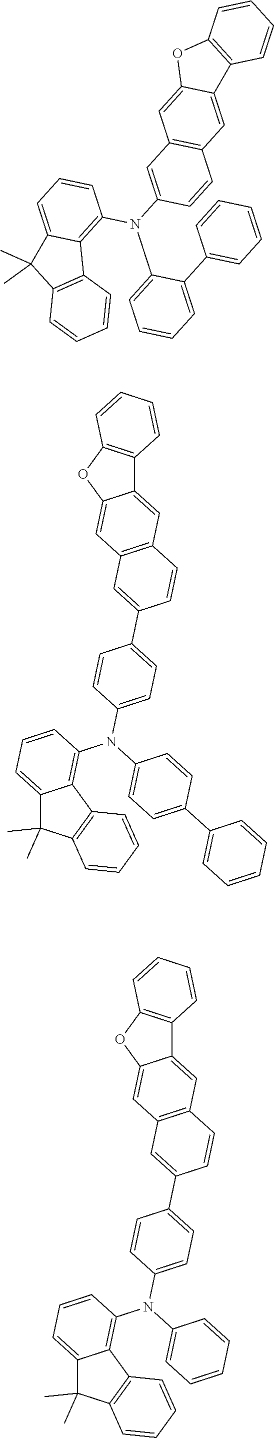 [SMILES: CC1(C)c2ccccc2-c2c(N(c3ccc4cc5c(cc4c3)oc3ccccc35)c3ccccc3-c3ccccc3)cccc21 ; CC1(C)c2ccccc2-c2c(N(c3ccc(-c4ccccc4)cc3)c3ccc(-c4ccc5cc6c(cc5c4)oc4ccccc46)cc3)cccc21 ; CC1(C)c2ccccc2-c2c(N(c3ccccc3)c3ccc(-c4ccc5cc6c(cc5c4)oc4ccccc46)cc3)cccc21] 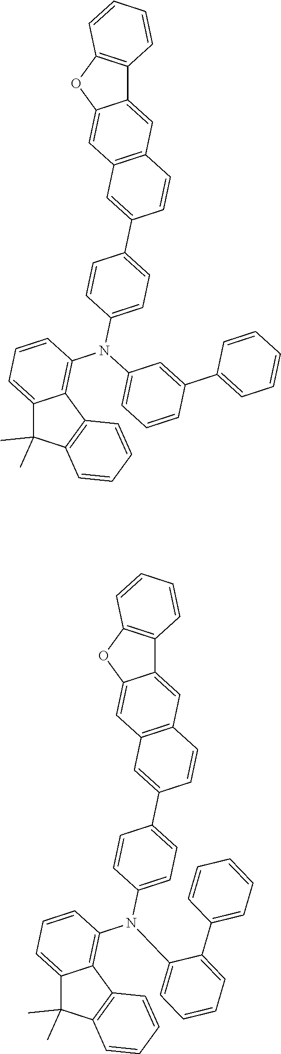 [SMILES: CC1(C)c2ccccc2-c2c(N(c3ccc(-c4ccc5cc6c(cc5c4)oc4ccccc46)cc3)c3cccc(-c4ccccc4)c3)cccc21 ; CC1(C)c2ccccc2-c2c(N(c3ccc(-c4ccc5cc6c(cc5c4)oc4ccccc46)cc3)c3ccccc3-c3ccccc3)cccc21] 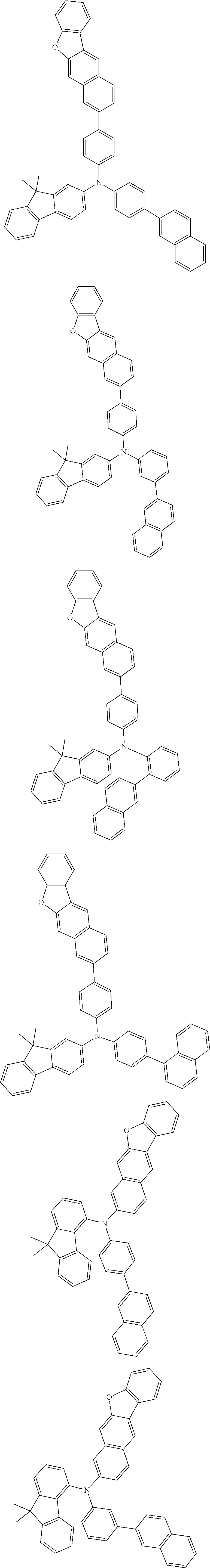 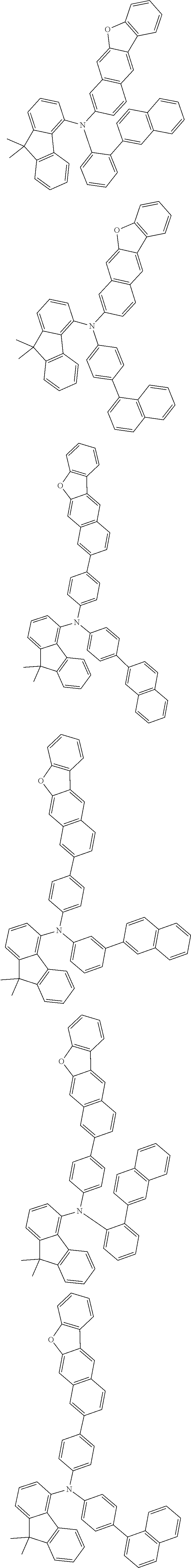 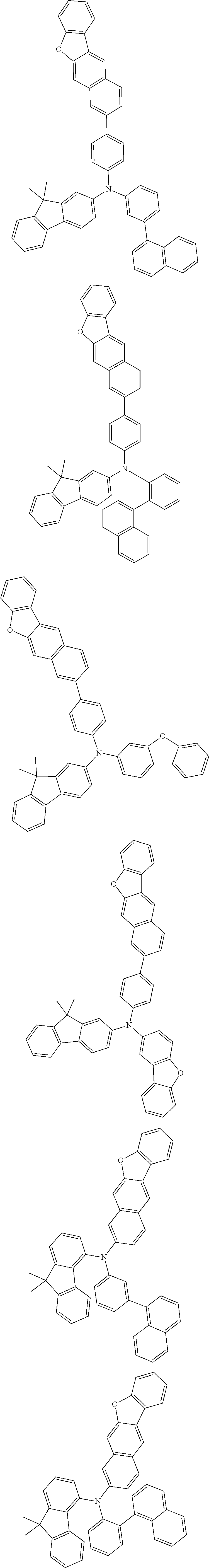 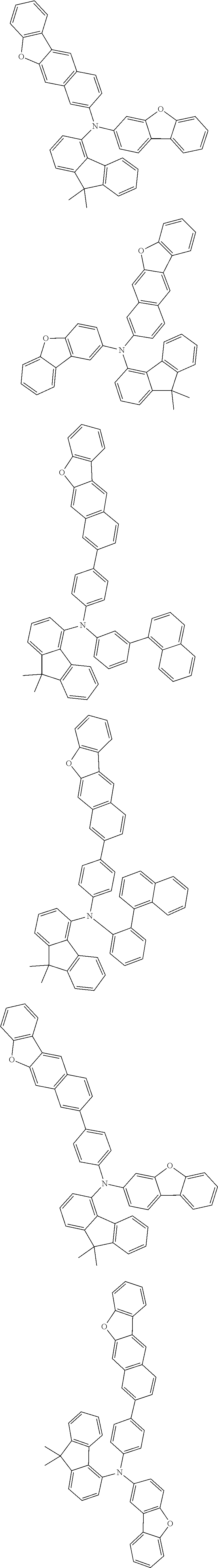 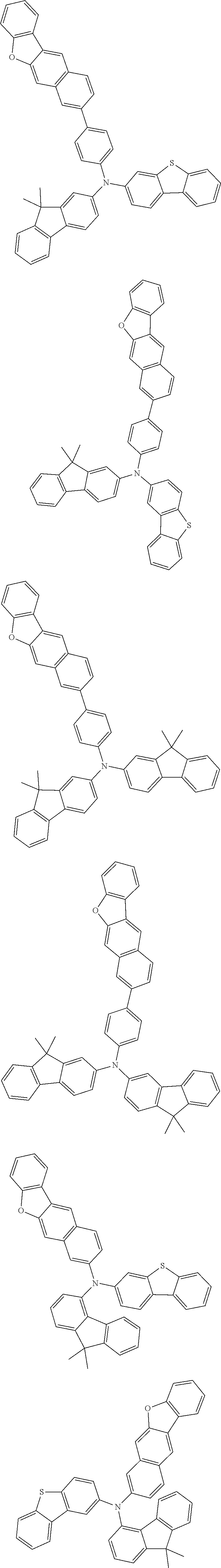 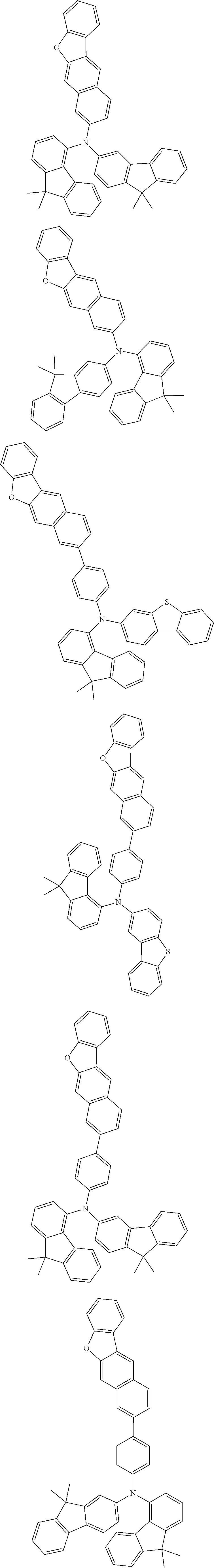 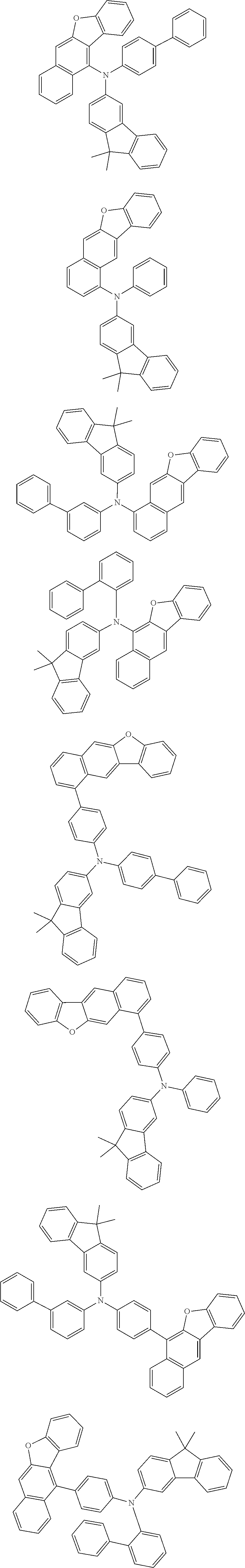 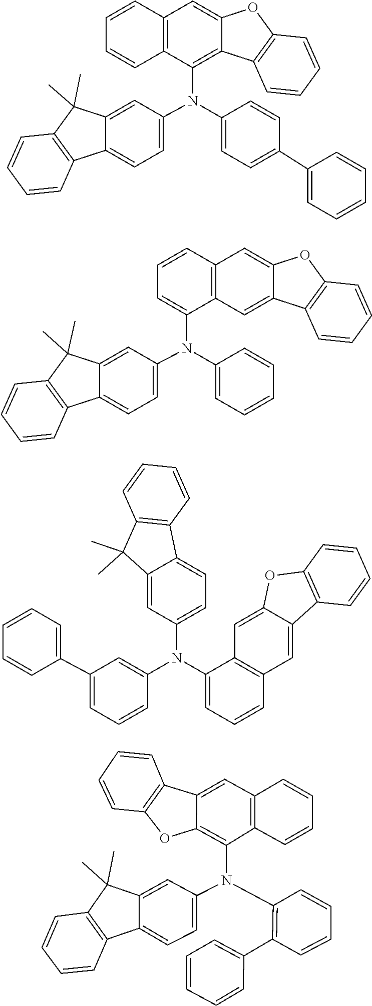 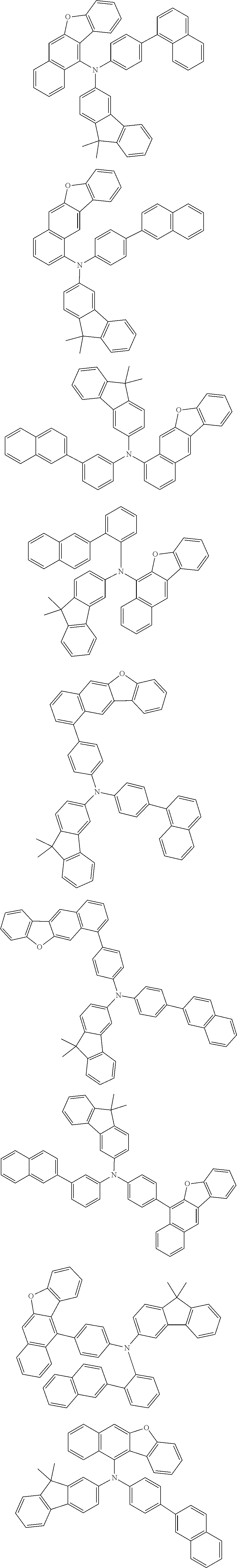 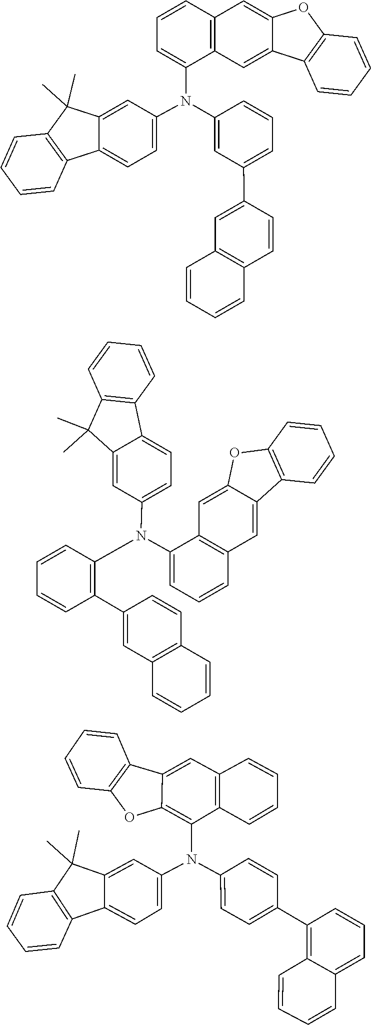 [SMILES: CC1(C)c2ccccc2-c2ccc(N(c3cccc(-c4ccc5ccccc5c4)c3)c3cccc4cc5oc6ccccc6c5cc34)cc21 ; CC1(C)c2ccccc2-c2ccc(N(c3ccccc3-c3ccc4ccccc4c3)c3cccc4cc5c(cc34)oc3ccccc35)cc21 ; CC1(C)c2ccccc2-c2ccc(N(c3ccc(-c4cccc5ccccc45)cc3)c3c4ccccc4cc4c3oc3ccccc34)cc21] 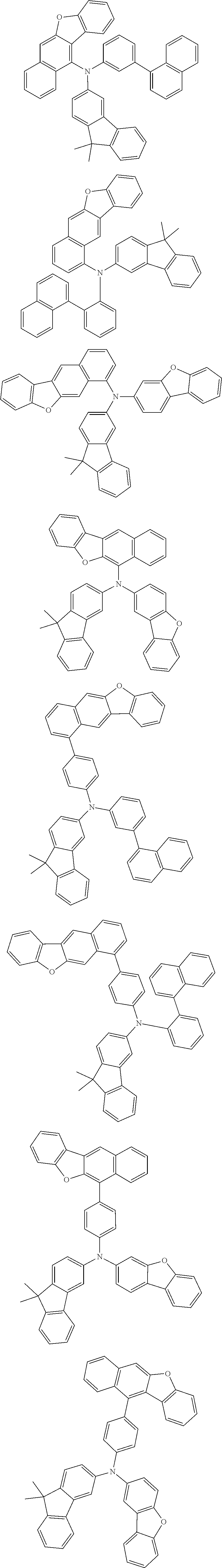 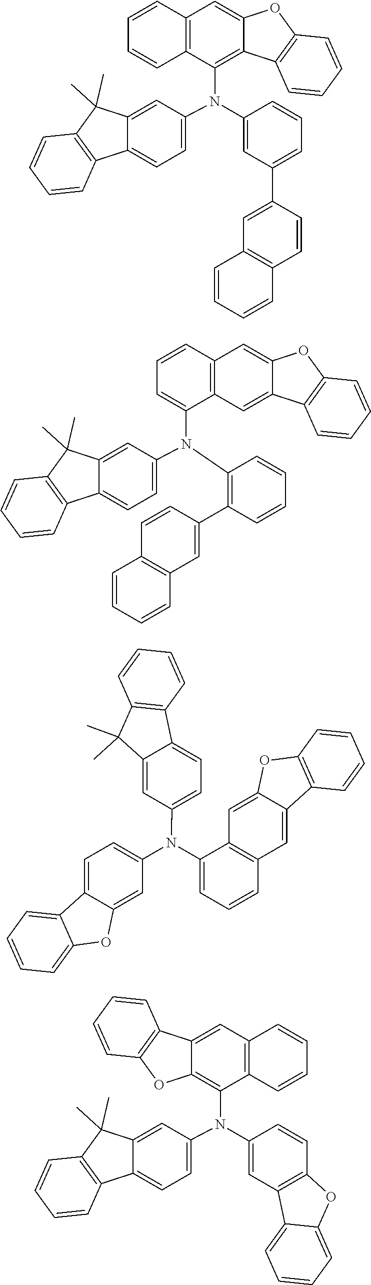 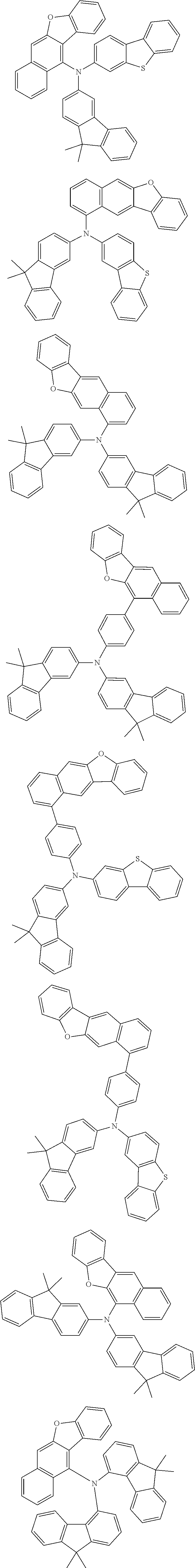 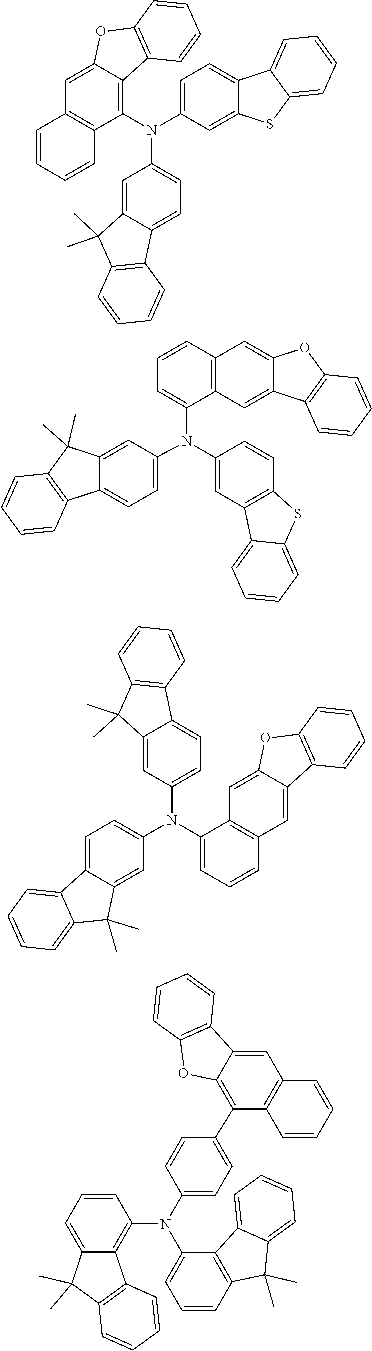 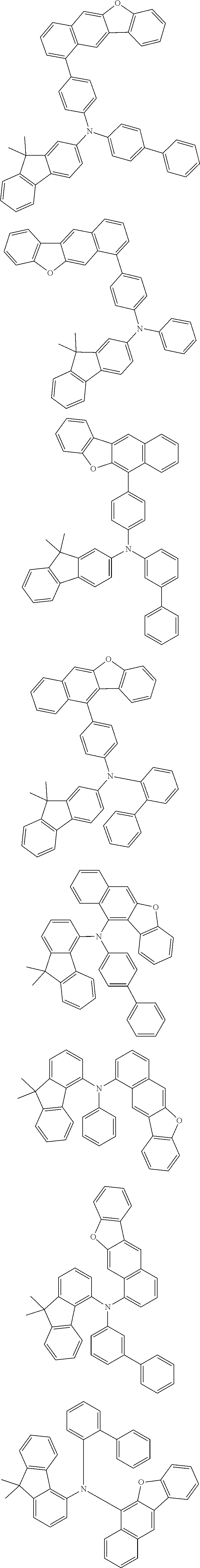 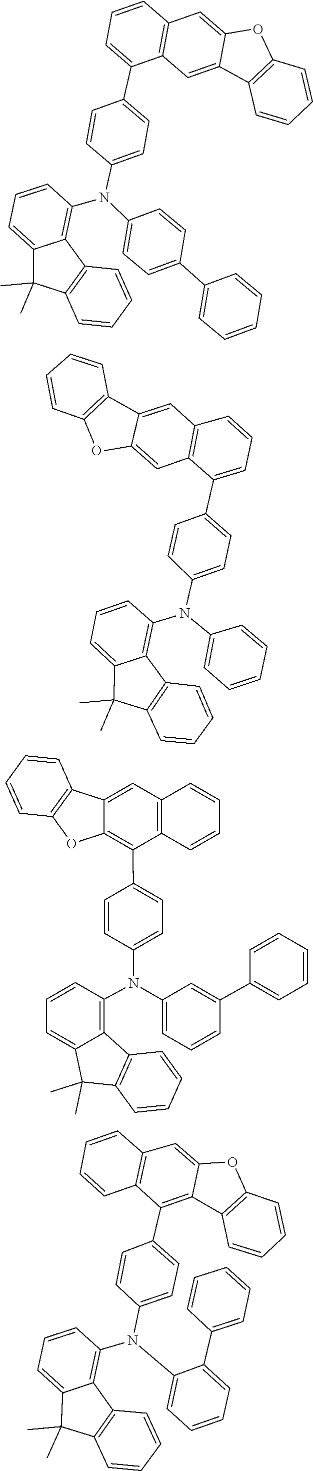 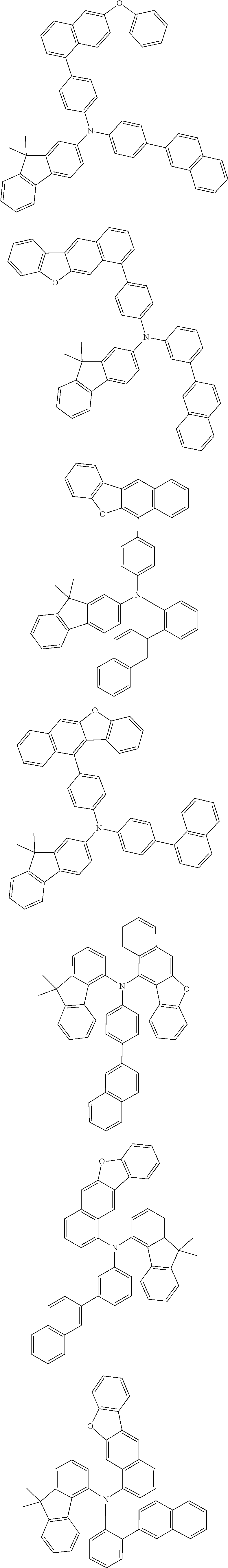 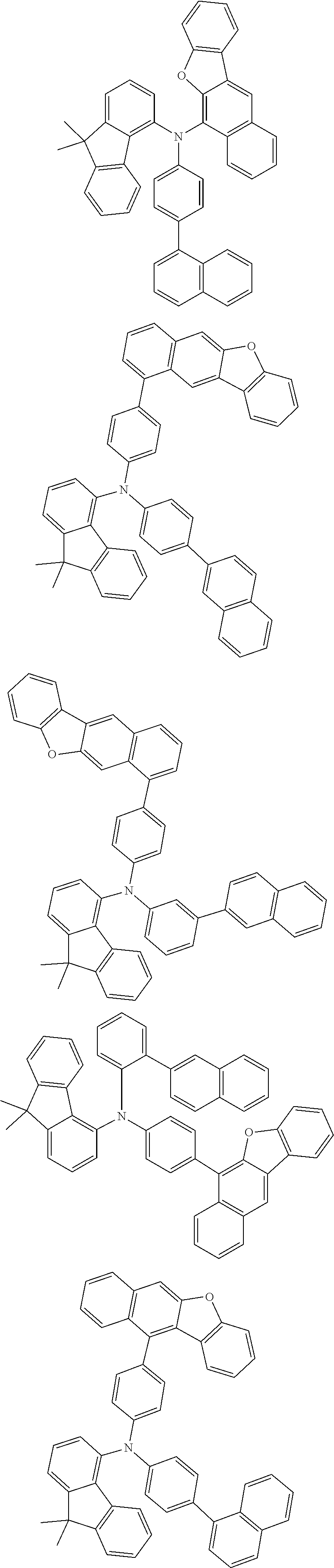 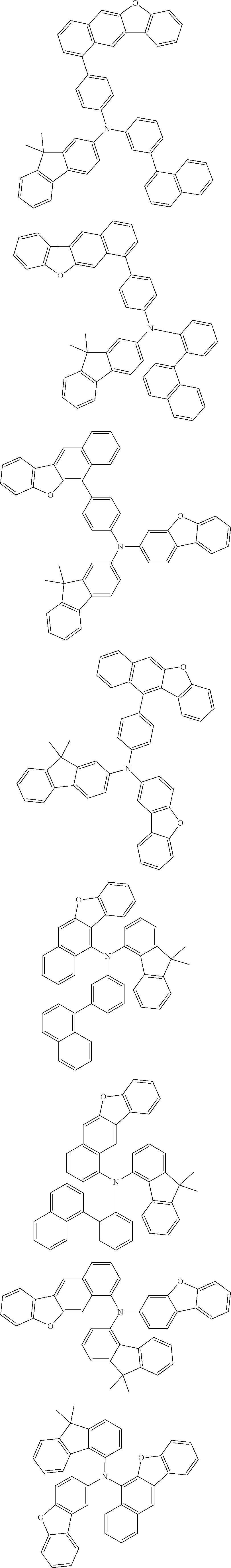 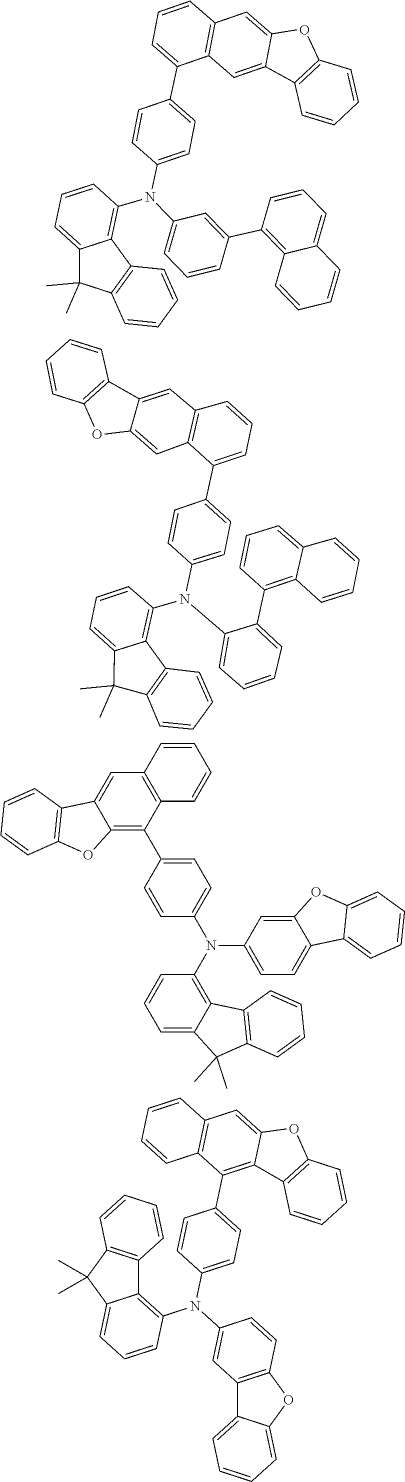 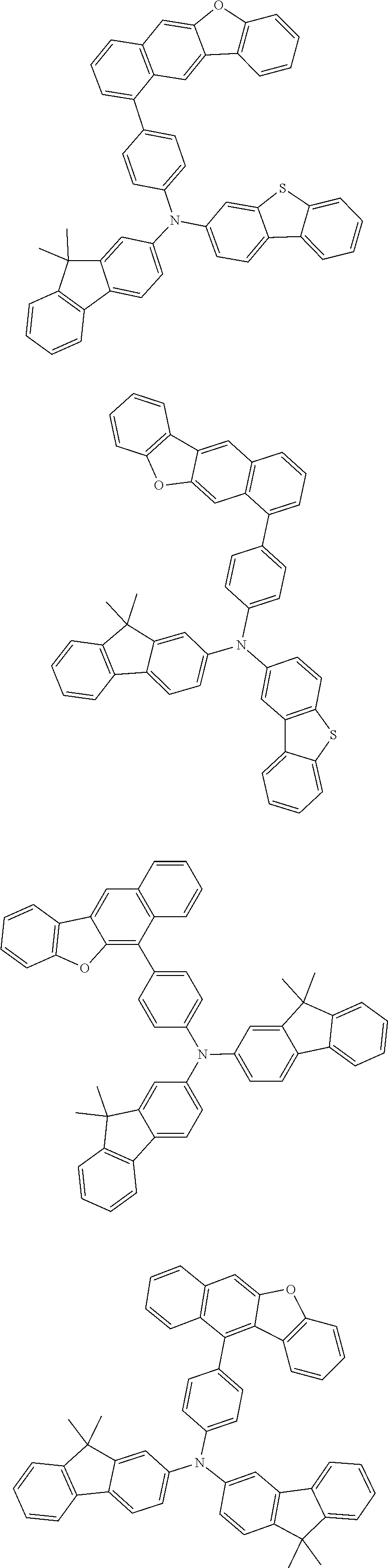 [SMILES: CC1(C)c2ccccc2-c2ccc(N(c3ccc(-c4cccc5cc6oc7ccccc7c6cc45)cc3)c3ccc4c(c3)sc3ccccc34)cc21 ; CC1(C)c2ccccc2-c2ccc(N(c3ccc(-c4cccc5cc6c(cc45)oc4ccccc46)cc3)c3ccc4sc5ccccc5c4c3)cc21 ; CC1(C)c2ccccc2-c2ccc(N(c3ccc(-c4c5ccccc5cc5c4oc4ccccc45)cc3)c3ccc4c(c3)C(C)(C)c3ccccc3-4)cc21 ; CC1(C)c2ccccc2-c2cc(N(c3ccc(-c4c5ccccc5cc5oc6ccccc6c45)cc3)c3ccc4c(c3)C(C)(C)c3ccccc3-4)ccc21] 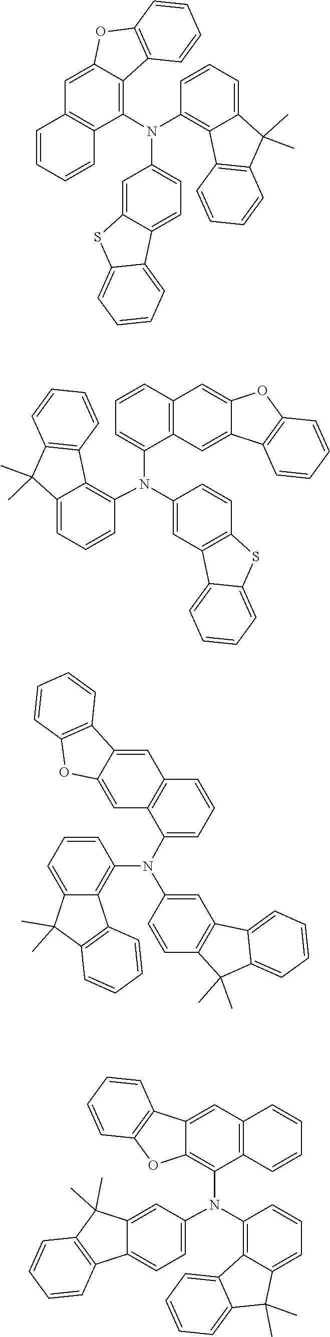 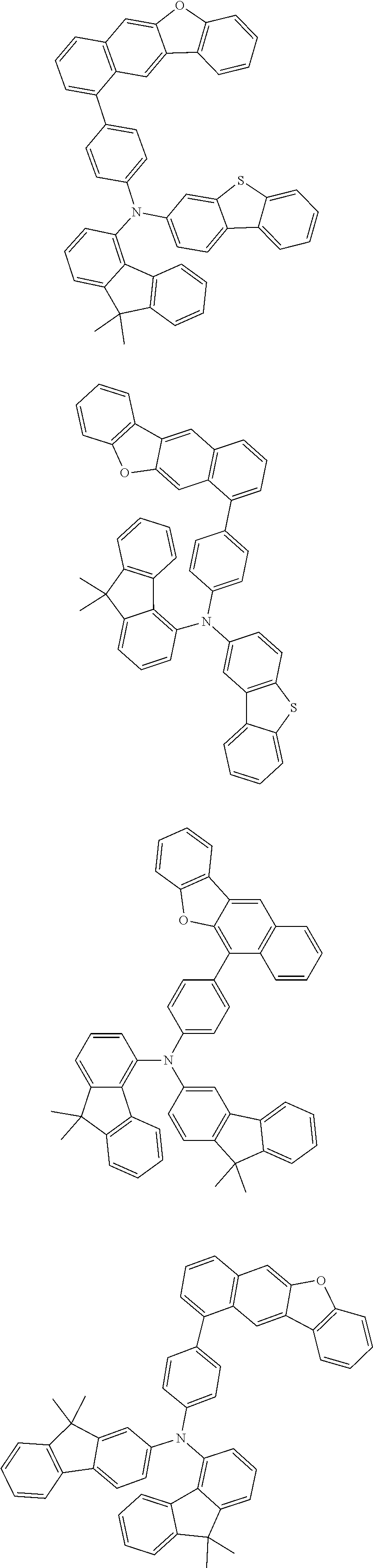 [SMILES: CC1(C)c2ccccc2-c2c(N(c3ccc(-c4cccc5cc6oc7ccccc7c6cc45)cc3)c3ccc4c(c3)sc3ccccc34)cccc21 ; CC1(C)c2ccccc2-c2c(N(c3ccc(-c4cccc5cc6c(cc45)oc4ccccc46)cc3)c3ccc4sc5ccccc5c4c3)cccc21 ; CC1(C)c2ccccc2-c2cc(N(c3ccc(-c4c5ccccc5cc5c4oc4ccccc45)cc3)c3cccc4c3-c3ccccc3C4(C)C)ccc21 ; CC1(C)c2ccccc2-c2ccc(N(c3ccc(-c4cccc5cc6oc7ccccc7c6cc45)cc3)c3cccc4c3-c3ccccc3C4(C)C)cc21] 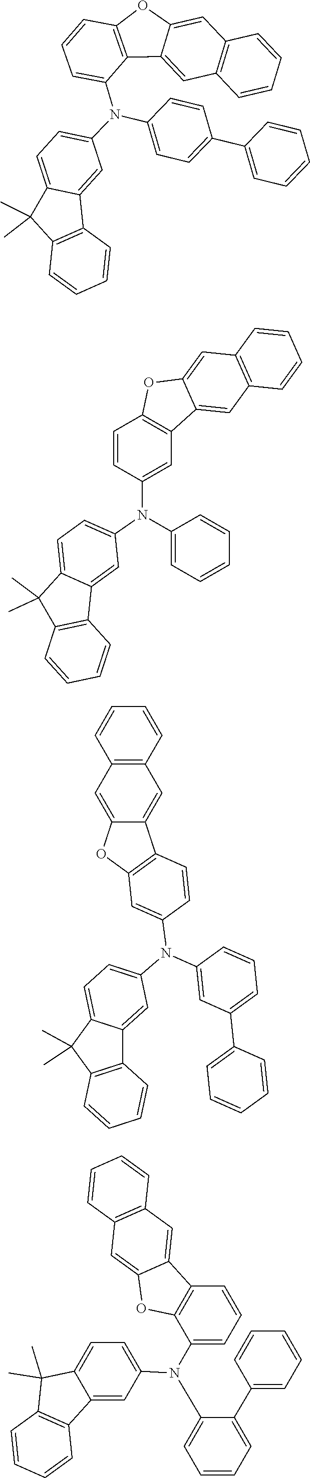 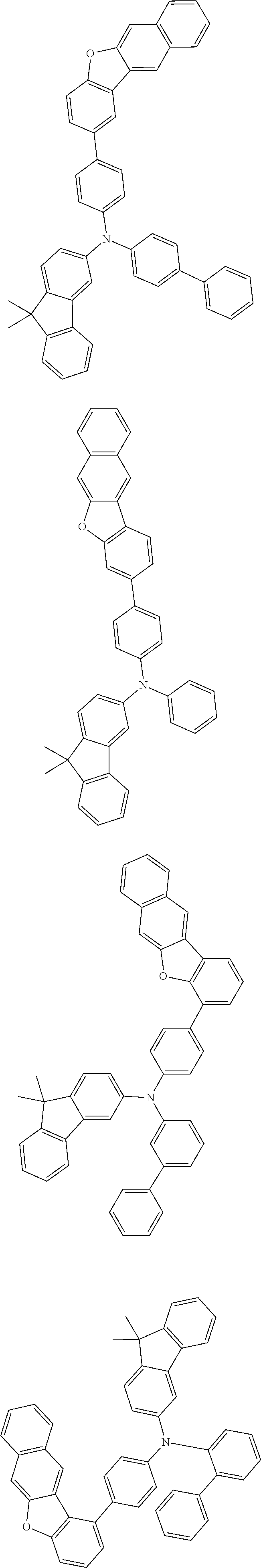 [SMILES: CC1(C)c2ccccc2-c2cc(N(c3ccc(-c4ccccc4)cc3)c3ccc(-c4ccc5oc6cc7ccccc7cc6c5c4)cc3)ccc21 ; CC1(C)c2ccccc2-c2cc(N(c3ccccc3)c3ccc(-c4ccc5c(c4)oc4cc6ccccc6cc45)cc3)ccc21 ; CC1(C)c2ccccc2-c2cc(N(c3ccc(-c4cccc5c4oc4cc6ccccc6cc45)cc3)c3cccc(-c4ccccc4)c3)ccc21 ; CC1(C)c2ccccc2-c2cc(N(c3ccc(-c4cccc5oc6cc7ccccc7cc6c45)cc3)c3ccccc3-c3ccccc3)ccc21] 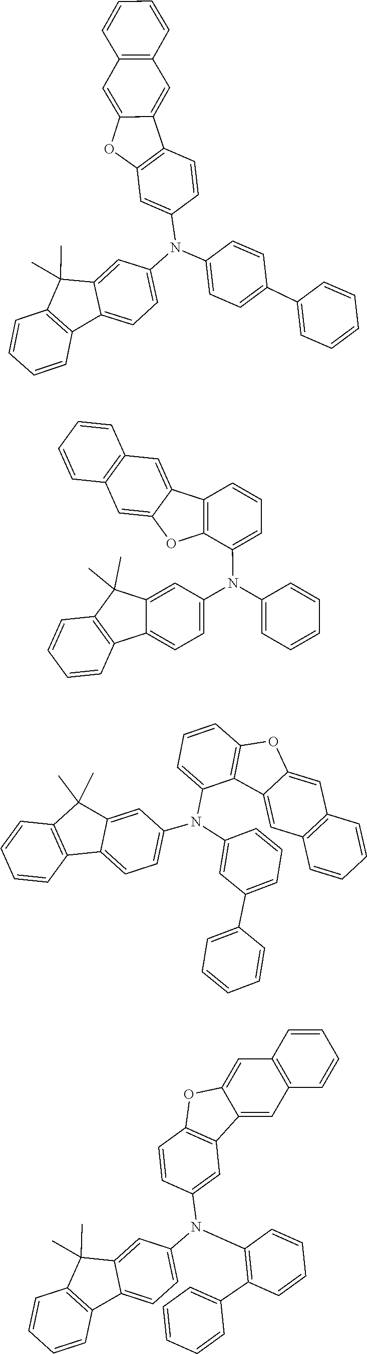 [SMILES: CC1(C)c2ccccc2-c2ccc(N(c3ccc(-c4ccccc4)cc3)c3ccc4c(c3)oc3cc5ccccc5cc34)cc21 ; CC1(C)c2ccccc2-c2ccc(N(c3ccccc3)c3cccc4c3oc3cc5ccccc5cc34)cc21 ; CC1(C)c2ccccc2-c2ccc(N(c3cccc(-c4ccccc4)c3)c3cccc4oc5cc6ccccc6cc5c34)cc21 ; CC1(C)c2ccccc2-c2ccc(N(c3ccc4oc5cc6ccccc6cc5c4c3)c3ccccc3-c3ccccc3)cc21] 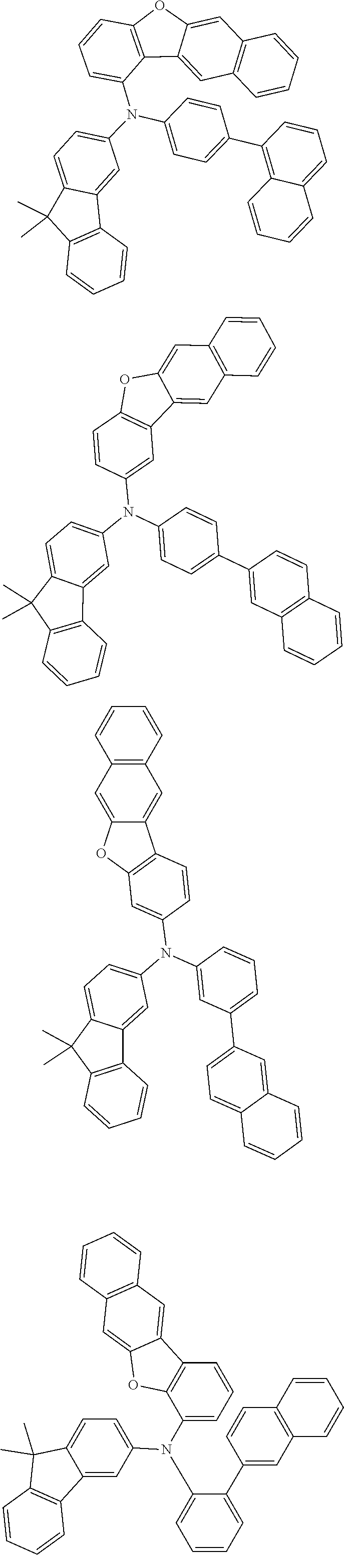 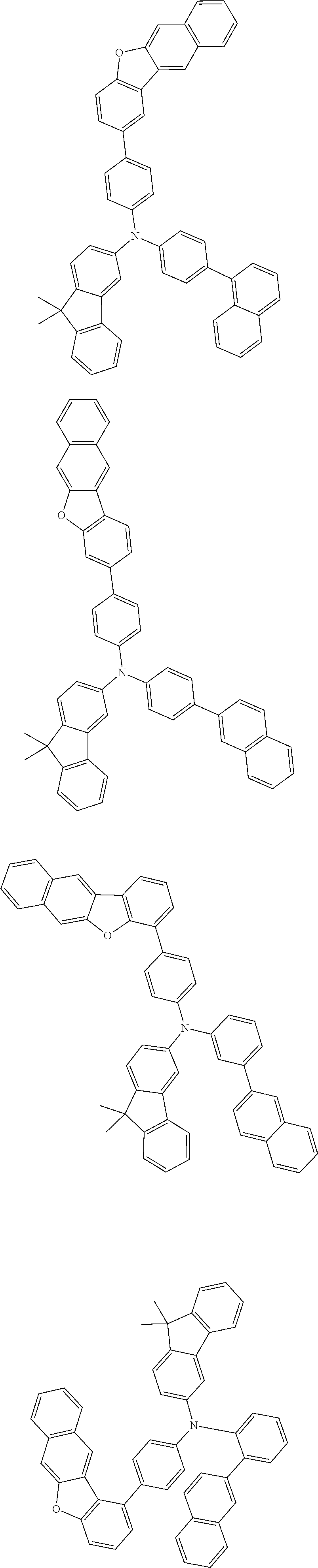 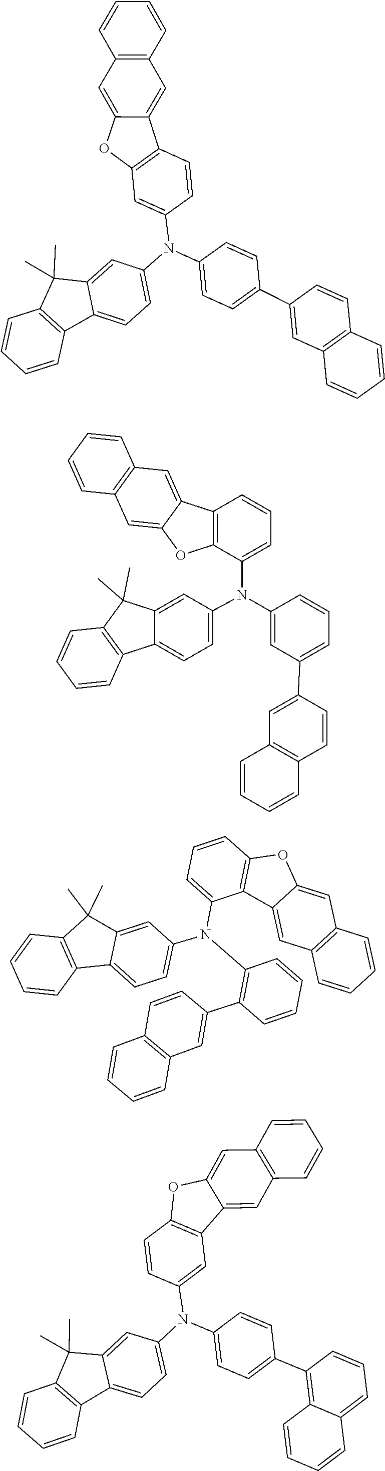 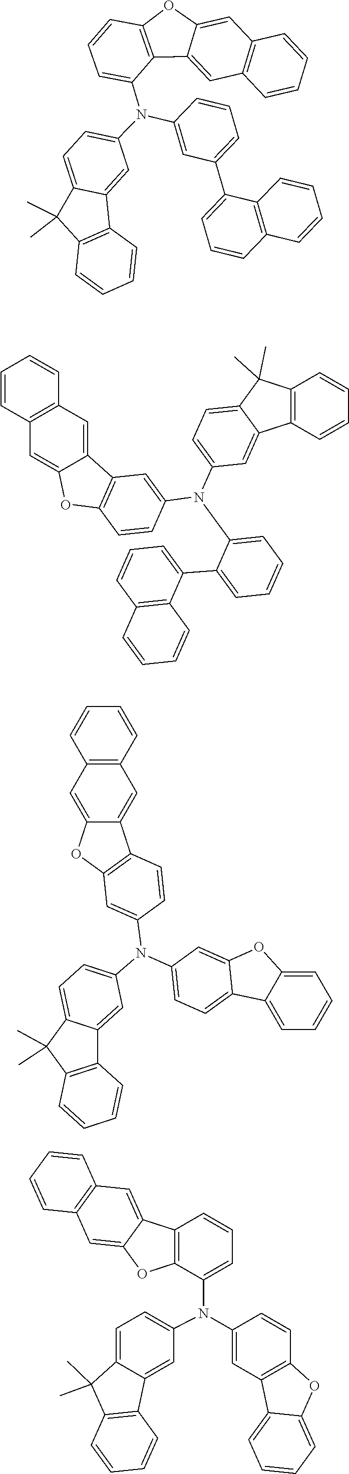 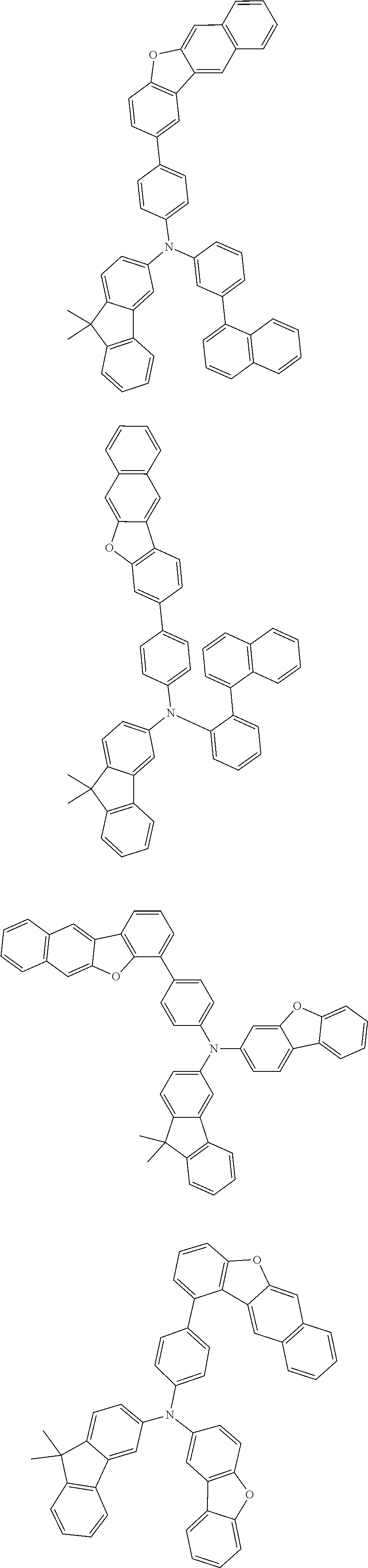 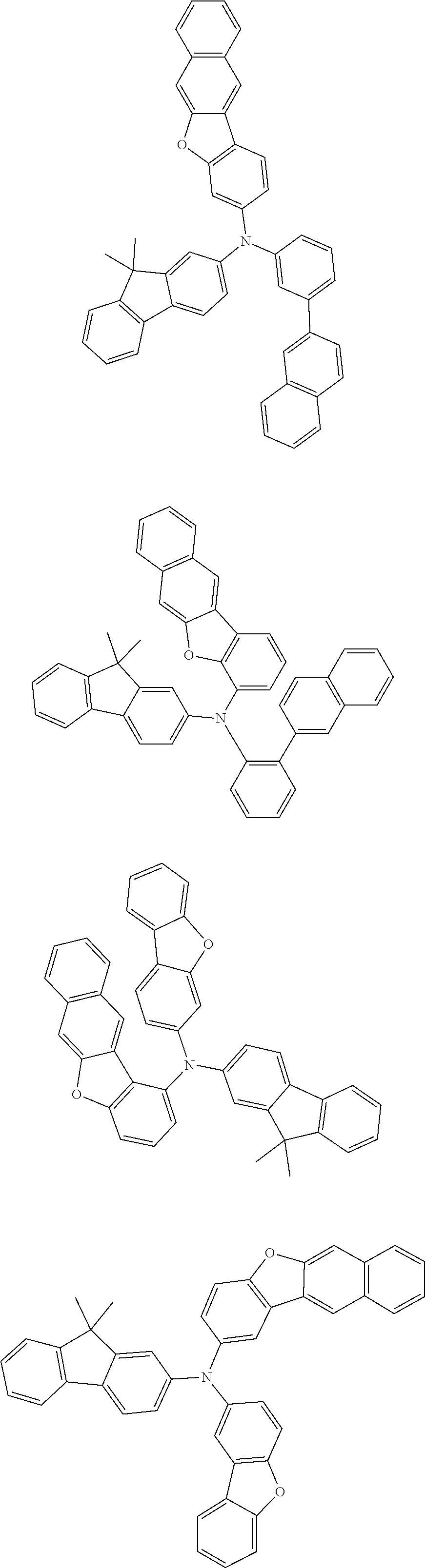 [SMILES: CC1(C)c2ccccc2-c2ccc(N(c3cccc(-c4ccc5ccccc5c4)c3)c3ccc4c(c3)oc3cc5ccccc5cc34)cc21 ; CC1(C)c2ccccc2-c2ccc(N(c3ccccc3-c3ccc4ccccc4c3)c3cccc4c3oc3cc5ccccc5cc34)cc21 ; CC1(C)c2ccccc2-c2ccc(N(c3ccc4c(c3)oc3ccccc34)c3cccc4oc5cc6ccccc6cc5c34)cc21 ; CC1(C)c2ccccc2-c2ccc(N(c3ccc4oc5ccccc5c4c3)c3ccc4oc5cc6ccccc6cc5c4c3)cc21] 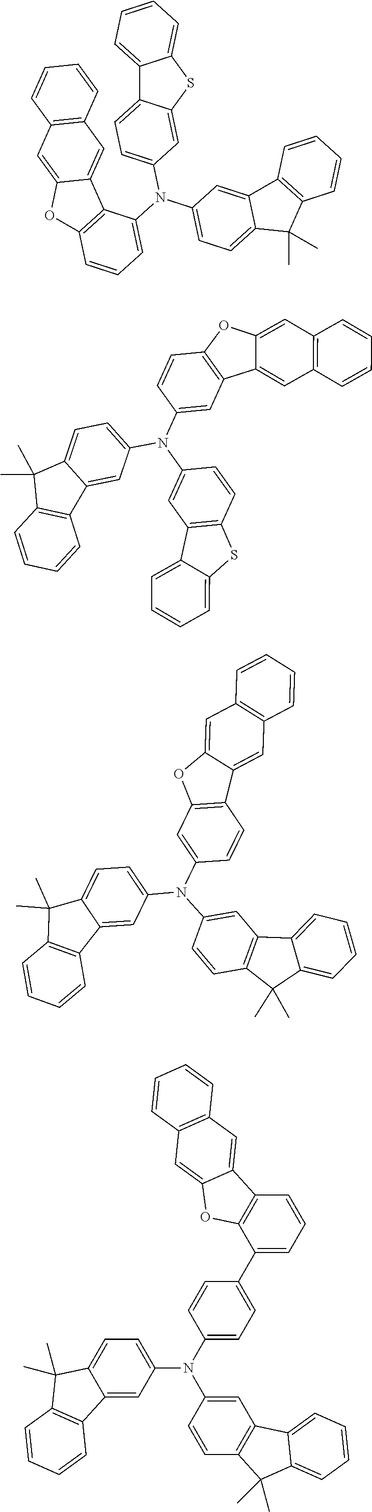 [SMILES: CC1(C)c2ccccc2-c2cc(N(c3ccc4c(c3)sc3ccccc34)c3cccc4oc5cc6ccccc6cc5c34)ccc21 ; CC1(C)c2ccccc2-c2cc(N(c3ccc4oc5cc6ccccc6cc5c4c3)c3ccc4sc5ccccc5c4c3)ccc21 ; CC1(C)c2ccccc2-c2cc(N(c3ccc4c(c3)-c3ccccc3C4(C)C)c3ccc4c(c3)oc3cc5ccccc5cc34)ccc21 ; CC1(C)c2ccccc2-c2cc(N(c3ccc(-c4cccc5c4oc4cc6ccccc6cc45)cc3)c3ccc4c(c3)-c3ccccc3C4(C)C)ccc21] 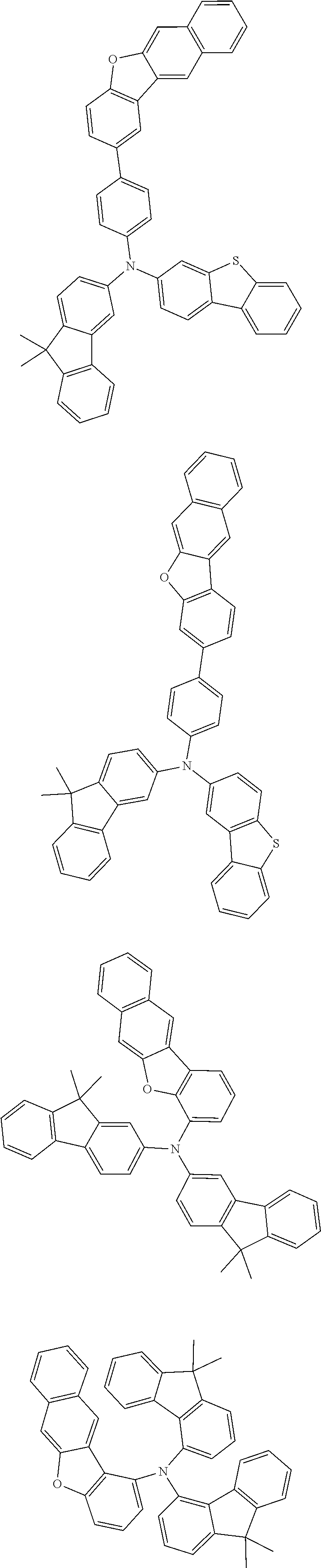 [SMILES: CC1(C)c2ccccc2-c2cc(N(c3ccc(-c4ccc5oc6cc7ccccc7cc6c5c4)cc3)c3ccc4c(c3)sc3ccccc34)ccc21 ; CC1(C)c2ccccc2-c2cc(N(c3ccc(-c4ccc5c(c4)oc4cc6ccccc6cc45)cc3)c3ccc4sc5ccccc5c4c3)ccc21 ; CC1(C)c2ccccc2-c2cc(N(c3ccc4c(c3)C(C)(C)c3ccccc3-4)c3cccc4c3oc3cc5ccccc5cc34)ccc21 ; CC1(C)c2ccccc2-c2c(N(c3cccc4c3-c3ccccc3C4(C)C)c3cccc4oc5cc6ccccc6cc5c34)cccc21] 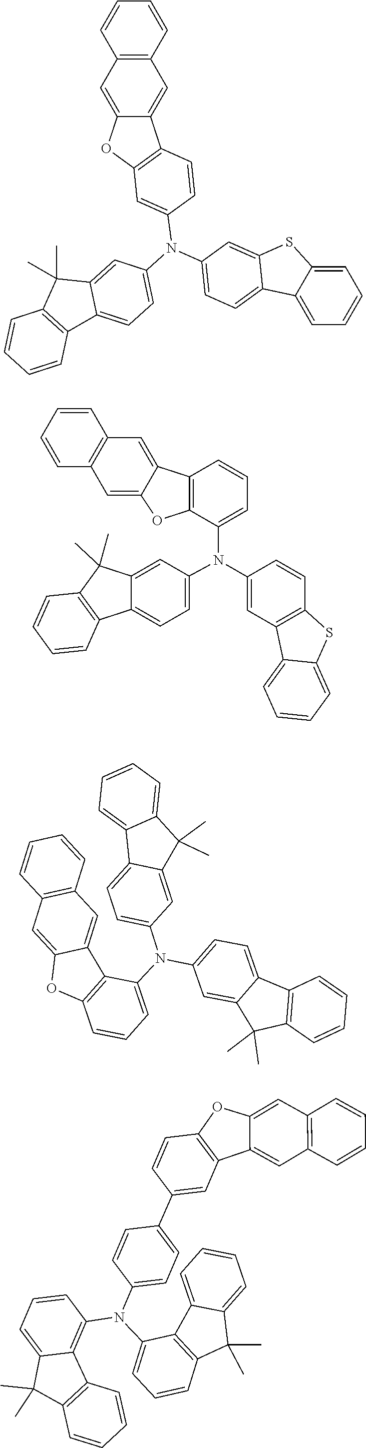 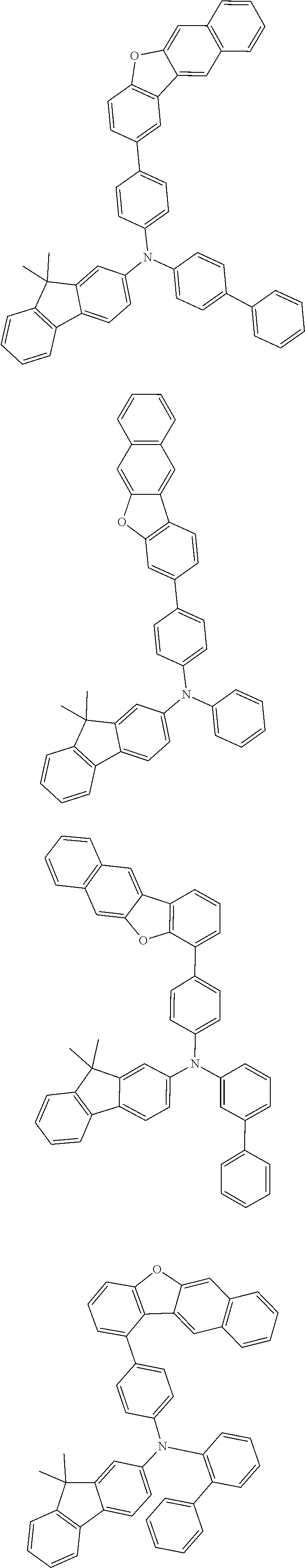 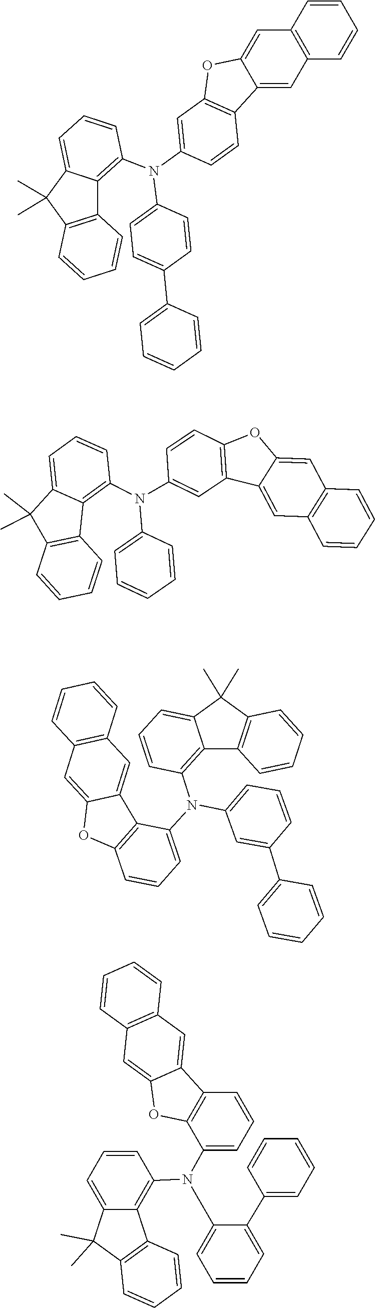 [SMILES: CC1(C)c2ccccc2-c2c(N(c3ccc(-c4ccccc4)cc3)c3ccc4c(c3)oc3cc5ccccc5cc34)cccc21 ; CC1(C)c2ccccc2-c2c(N(c3ccccc3)c3ccc4oc5cc6ccccc6cc5c4c3)cccc21 ; CC1(C)c2ccccc2-c2c(N(c3cccc(-c4ccccc4)c3)c3cccc4oc5cc6ccccc6cc5c34)cccc21 ; CC1(C)c2ccccc2-c2c(N(c3ccccc3-c3ccccc3)c3cccc4c3oc3cc5ccccc5cc34)cccc21] 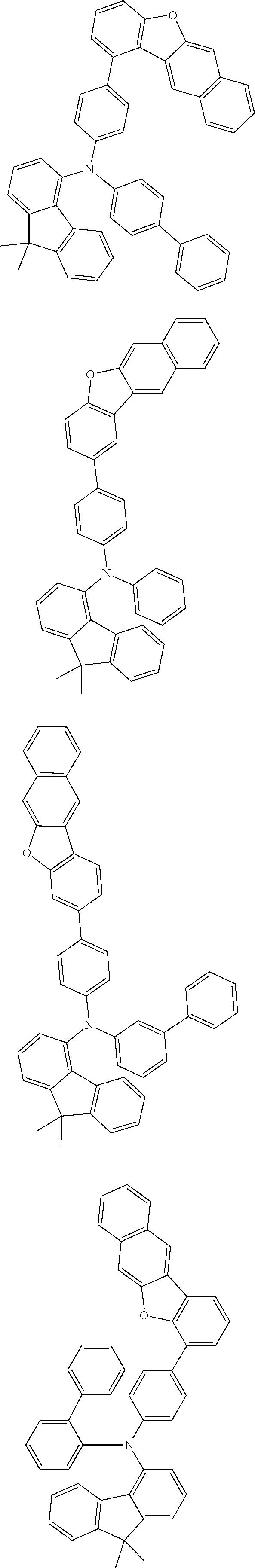 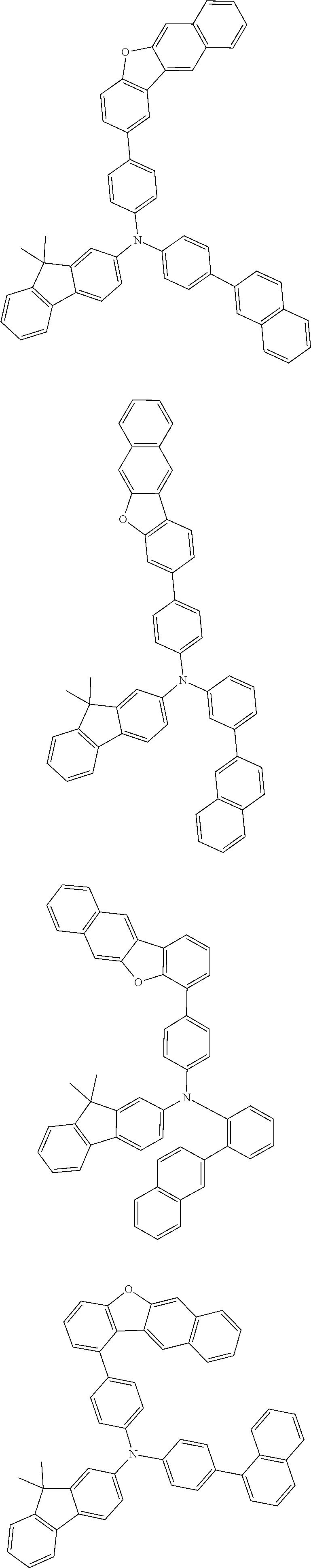 [SMILES: CC1(C)c2ccccc2-c2ccc(N(c3ccc(-c4ccc5ccccc5c4)cc3)c3ccc(-c4ccc5oc6cc7ccccc7cc6c5c4)cc3)cc21 ; CC1(C)c2ccccc2-c2ccc(N(c3ccc(-c4ccc5c(c4)oc4cc6ccccc6cc45)cc3)c3cccc(-c4ccc5ccccc5c4)c3)cc21 ; CC1(C)c2ccccc2-c2ccc(N(c3ccc(-c4cccc5c4oc4cc6ccccc6cc45)cc3)c3ccccc3-c3ccc4ccccc4c3)cc21 ; CC1(C)c2ccccc2-c2ccc(N(c3ccc(-c4cccc5ccccc45)cc3)c3ccc(-c4cccc5oc6cc7ccccc7cc6c45)cc3)cc21] 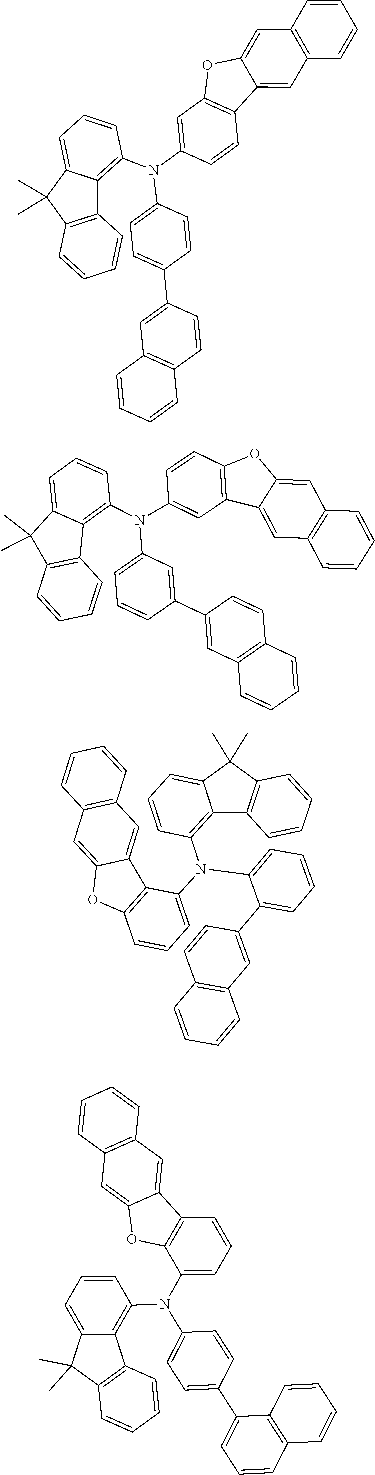 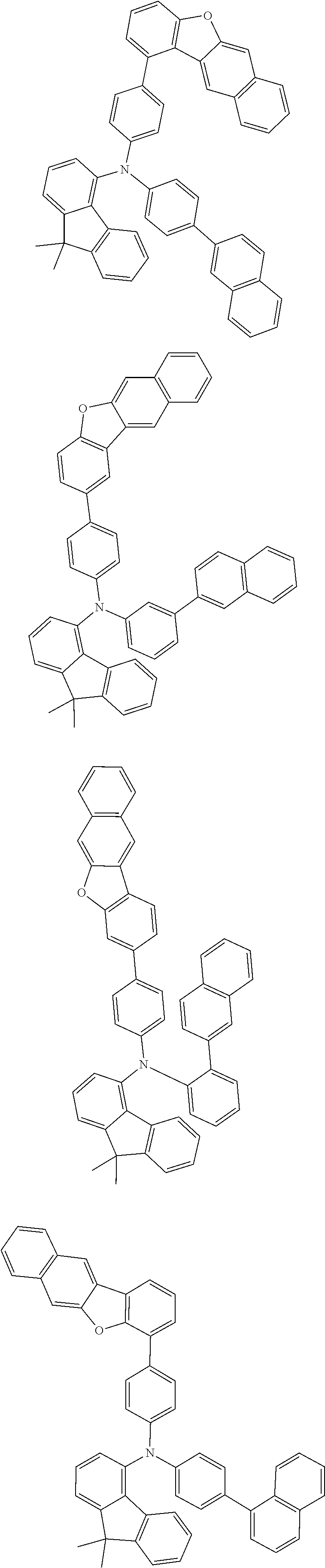 [SMILES: CC1(C)c2ccccc2-c2c(N(c3ccc(-c4ccc5ccccc5c4)cc3)c3ccc(-c4cccc5oc6cc7ccccc7cc6c45)cc3)cccc21 ; CC1(C)c2ccccc2-c2c(N(c3ccc(-c4ccc5oc6cc7ccccc7cc6c5c4)cc3)c3cccc(-c4ccc5ccccc5c4)c3)cccc21 ; CC1(C)c2ccccc2-c2c(N(c3ccc(-c4ccc5c(c4)oc4cc6ccccc6cc45)cc3)c3ccccc3-c3ccc4ccccc4c3)cccc21 ; CC1(C)c2ccccc2-c2c(N(c3ccc(-c4cccc5ccccc45)cc3)c3ccc(-c4cccc5c4oc4cc6ccccc6cc45)cc3)cccc21] 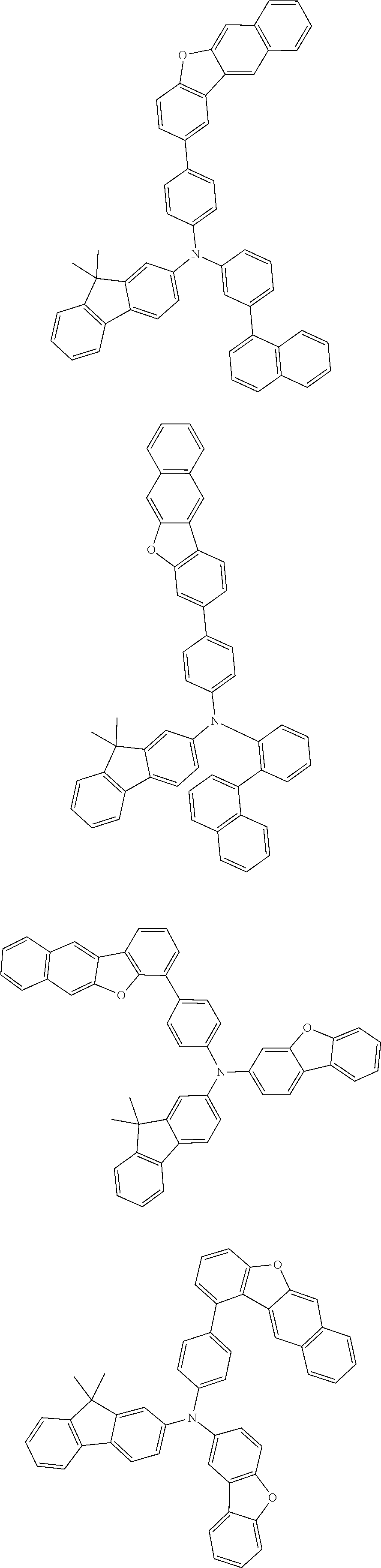 [SMILES: CC1(C)c2ccccc2-c2ccc(N(c3ccc(-c4ccc5oc6cc7ccccc7cc6c5c4)cc3)c3cccc(-c4cccc5ccccc45)c3)cc21 ; CC1(C)c2ccccc2-c2ccc(N(c3ccc(-c4ccc5c(c4)oc4cc6ccccc6cc45)cc3)c3ccccc3-c3cccc4ccccc34)cc21 ; CC1(C)c2ccccc2-c2ccc(N(c3ccc(-c4cccc5c4oc4cc6ccccc6cc45)cc3)c3ccc4c(c3)oc3ccccc34)cc21 ; CC1(C)c2ccccc2-c2ccc(N(c3ccc(-c4cccc5oc6cc7ccccc7cc6c45)cc3)c3ccc4oc5ccccc5c4c3)cc21] 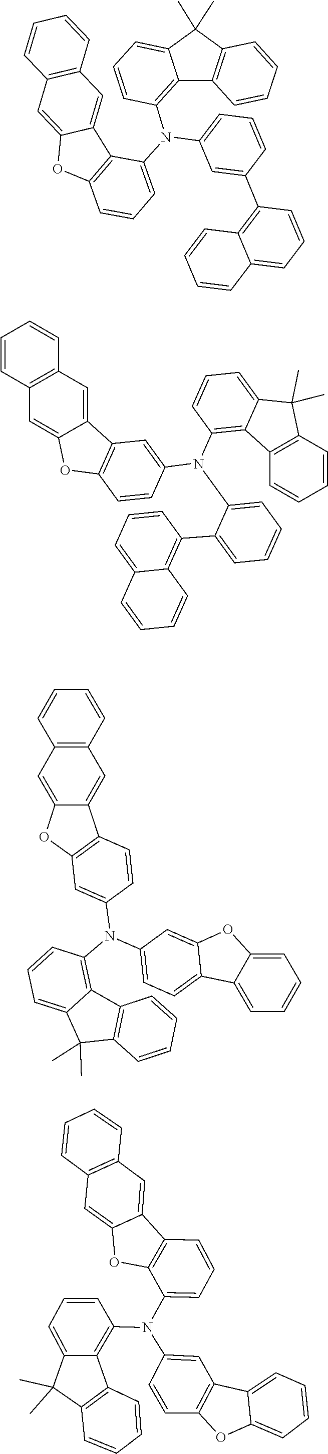 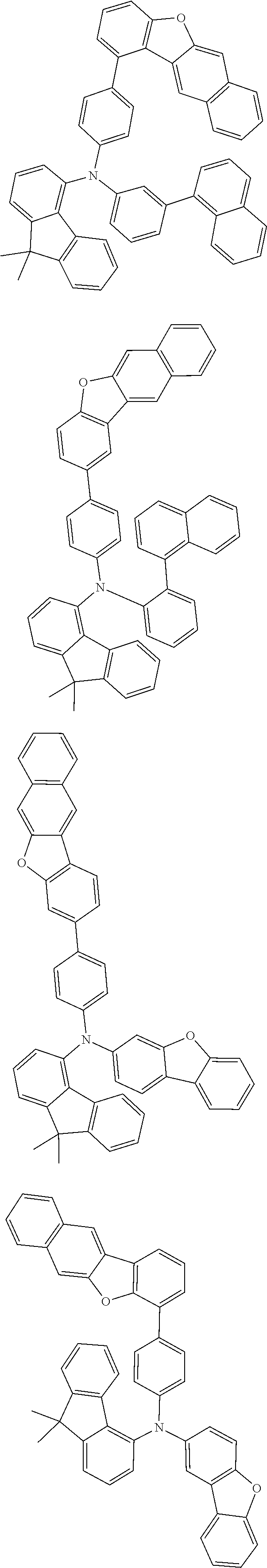 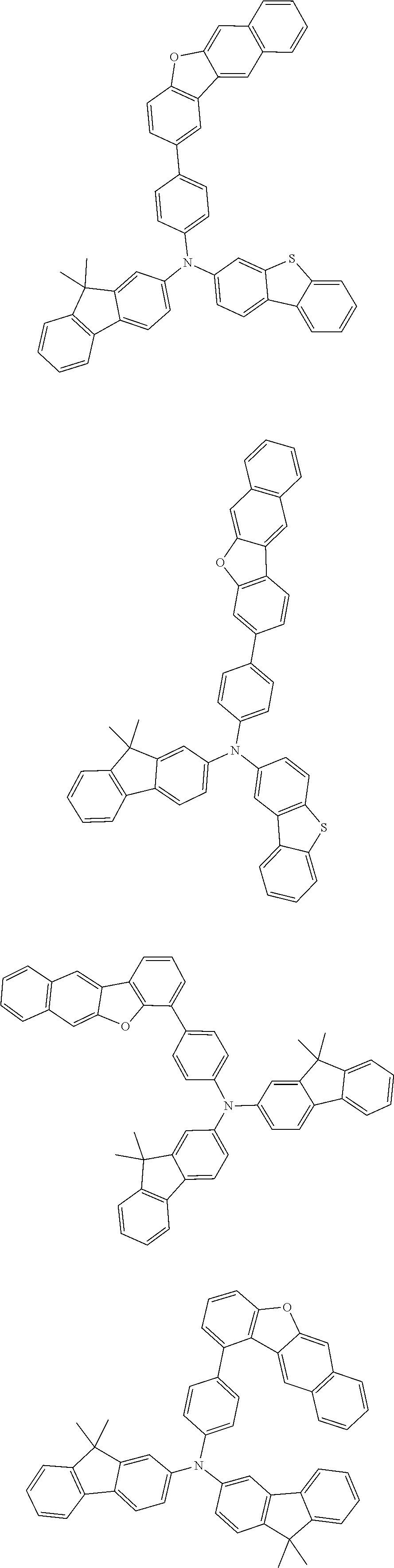 [SMILES: CC1(C)c2ccccc2-c2ccc(N(c3ccc(-c4ccc5oc6cc7ccccc7cc6c5c4)cc3)c3ccc4c(c3)sc3ccccc34)cc21 ; CC1(C)c2ccccc2-c2ccc(N(c3ccc(-c4ccc5c(c4)oc4cc6ccccc6cc45)cc3)c3ccc4sc5ccccc5c4c3)cc21 ; CC1(C)c2ccccc2-c2ccc(N(c3ccc(-c4cccc5c4oc4cc6ccccc6cc45)cc3)c3ccc4c(c3)C(C)(C)c3ccccc3-4)cc21 ; CC1(C)c2ccccc2-c2cc(N(c3ccc(-c4cccc5oc6cc7ccccc7cc6c45)cc3)c3ccc4c(c3)C(C)(C)c3ccccc3-4)ccc21] 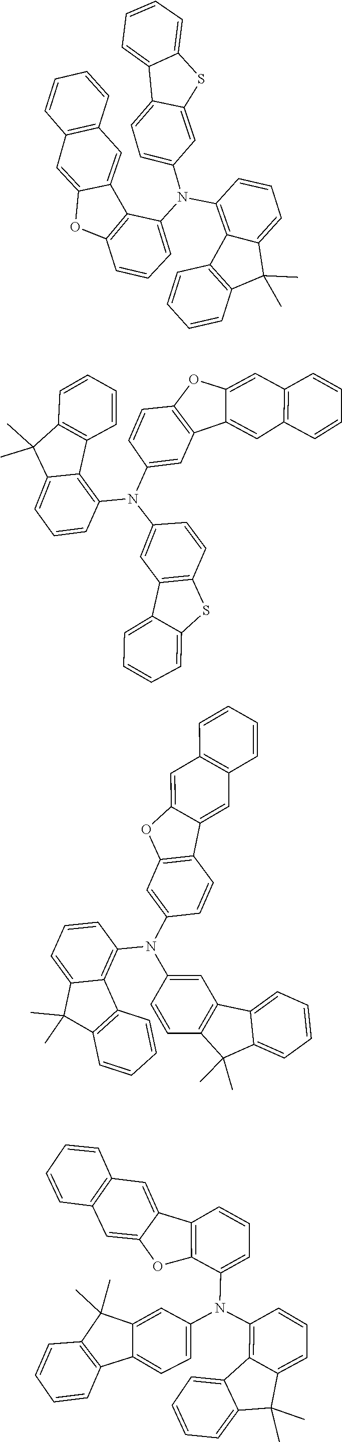 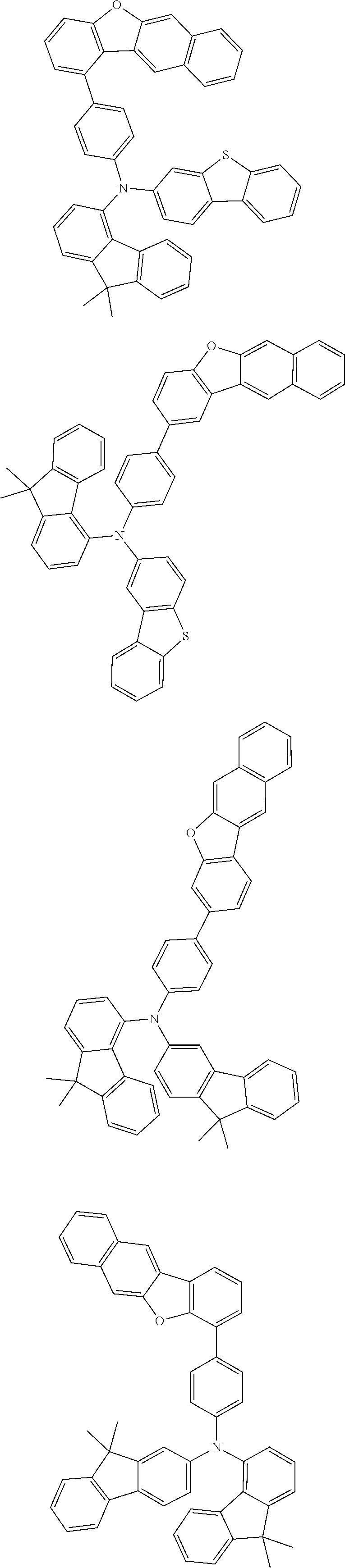 [SMILES: CC1(C)c2ccccc2-c2c(N(c3ccc(-c4cccc5oc6cc7ccccc7cc6c45)cc3)c3ccc4c(c3)sc3ccccc34)cccc21 ; CC1(C)c2ccccc2-c2c(N(c3ccc(-c4ccc5oc6cc7ccccc7cc6c5c4)cc3)c3ccc4sc5ccccc5c4c3)cccc21 ; CC1(C)c2ccccc2-c2cc(N(c3ccc(-c4ccc5c(c4)oc4cc6ccccc6cc45)cc3)c3cccc4c3-c3ccccc3C4(C)C)ccc21 ; CC1(C)c2ccccc2-c2ccc(N(c3ccc(-c4cccc5c4oc4cc6ccccc6cc45)cc3)c3cccc4c3-c3ccccc3C4(C)C)cc21] 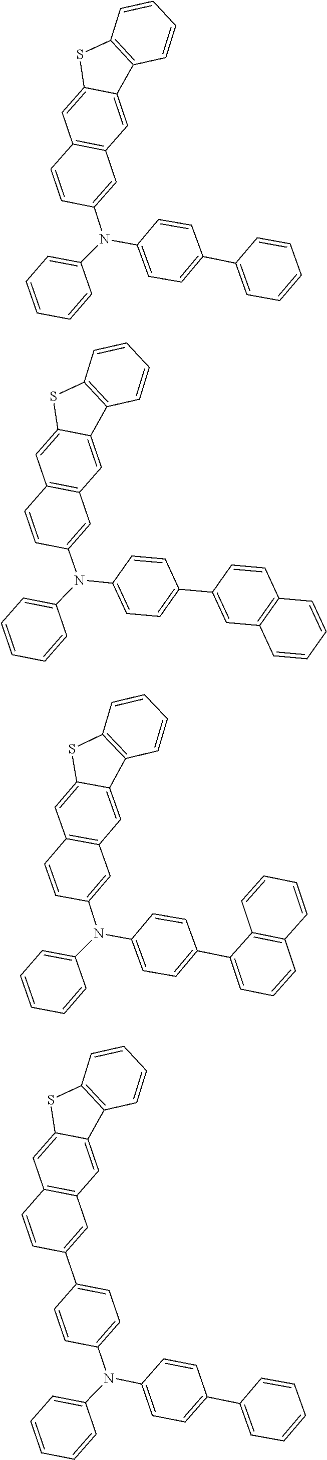 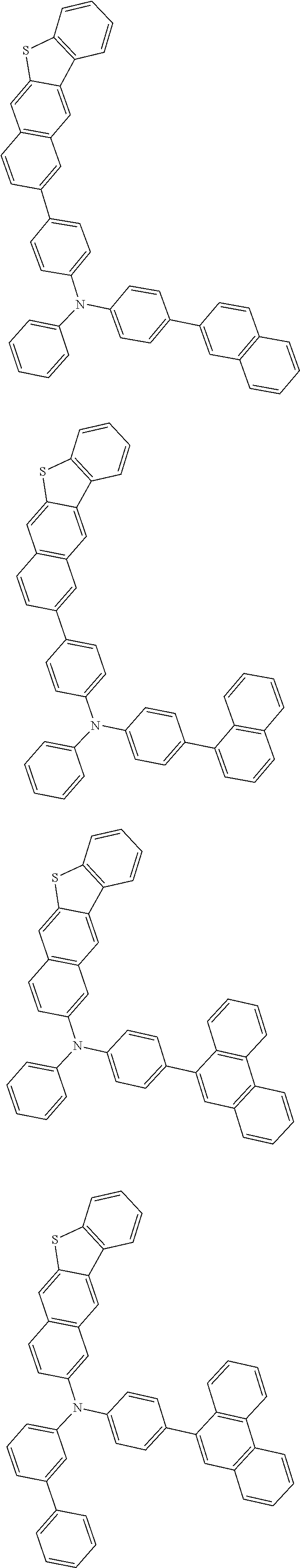 [SMILES: c1ccc(N(c2ccc(-c3ccc4ccccc4c3)cc2)c2ccc(-c3ccc4cc5sc6ccccc6c5cc4c3)cc2)cc1 ; c1ccc(N(c2ccc(-c3ccc4cc5sc6ccccc6c5cc4c3)cc2)c2ccc(-c3cccc4ccccc34)cc2)cc1 ; c1ccc(N(c2ccc(-c3cc4ccccc4c4ccccc34)cc2)c2ccc3cc4sc5ccccc5c4cc3c2)cc1 ; c1ccc(-c2cccc(N(c3ccc(-c4cc5ccccc5c5ccccc45)cc3)c3ccc4cc5sc6ccccc6c5cc4c3)c2)cc1] 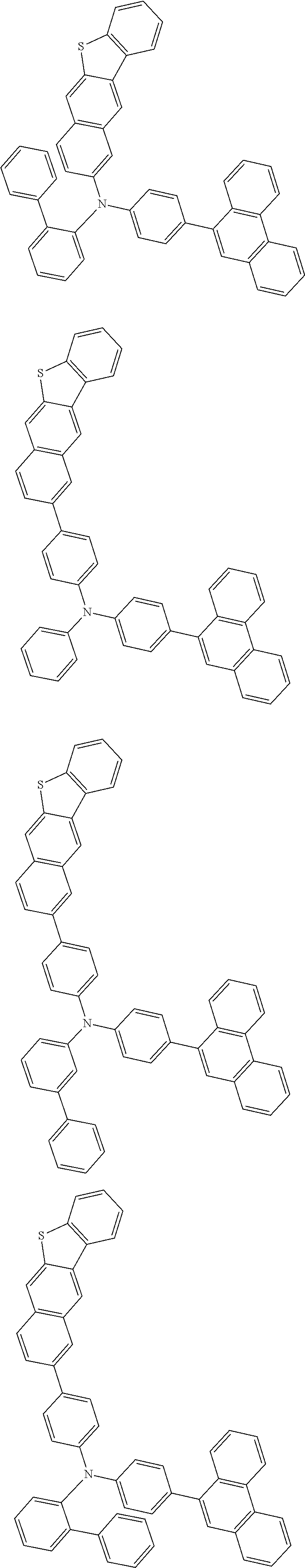 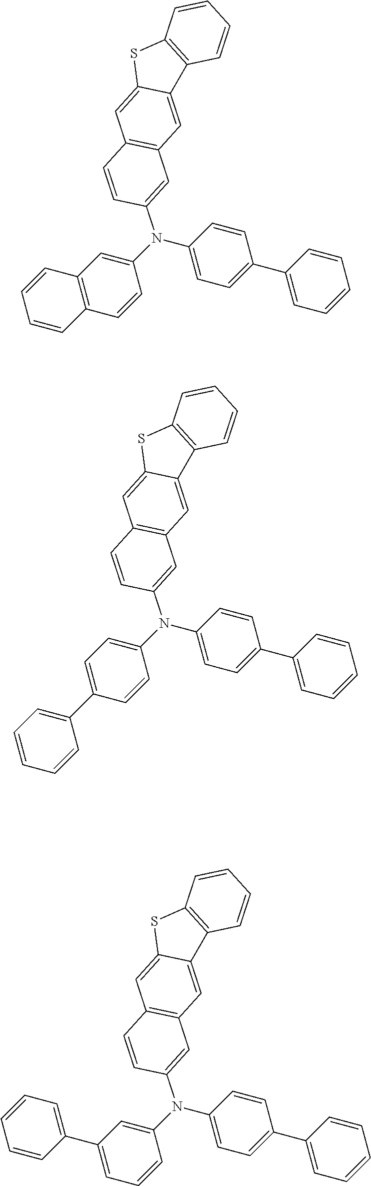 [SMILES: c1ccc(-c2ccc(N(c3ccc4ccccc4c3)c3ccc4cc5sc6ccccc6c5cc4c3)cc2)cc1 ; c1ccc(-c2ccc(N(c3ccc(-c4ccccc4)cc3)c3ccc4cc5sc6ccccc6c5cc4c3)cc2)cc1 ; c1ccc(-c2ccc(N(c3cccc(-c4ccccc4)c3)c3ccc4cc5sc6ccccc6c5cc4c3)cc2)cc1] 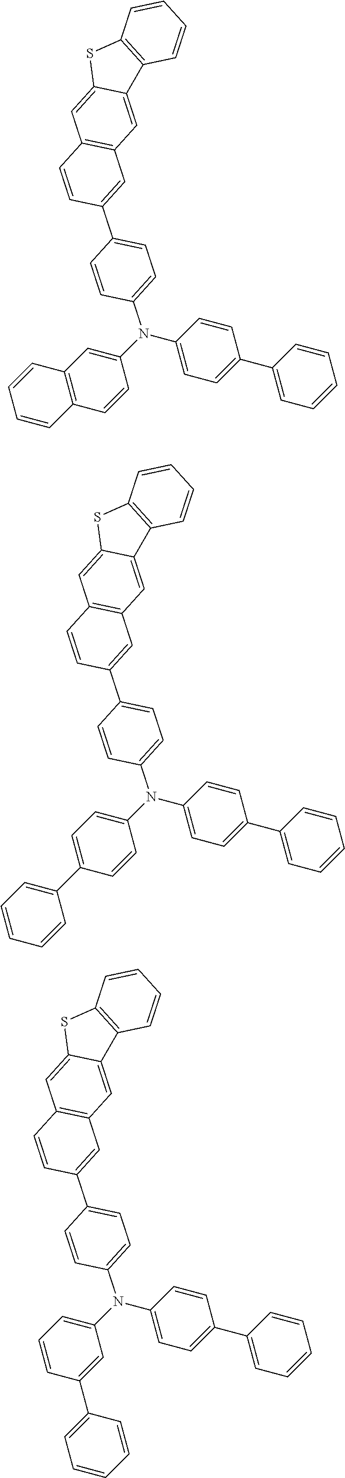 [SMILES: c1ccc(-c2ccc(N(c3ccc(-c4ccc5cc6sc7ccccc7c6cc5c4)cc3)c3ccc4ccccc4c3)cc2)cc1 ; c1ccc(-c2ccc(N(c3ccc(-c4ccccc4)cc3)c3ccc(-c4ccc5cc6sc7ccccc7c6cc5c4)cc3)cc2)cc1 ; c1ccc(-c2ccc(N(c3ccc(-c4ccc5cc6sc7ccccc7c6cc5c4)cc3)c3cccc(-c4ccccc4)c3)cc2)cc1] 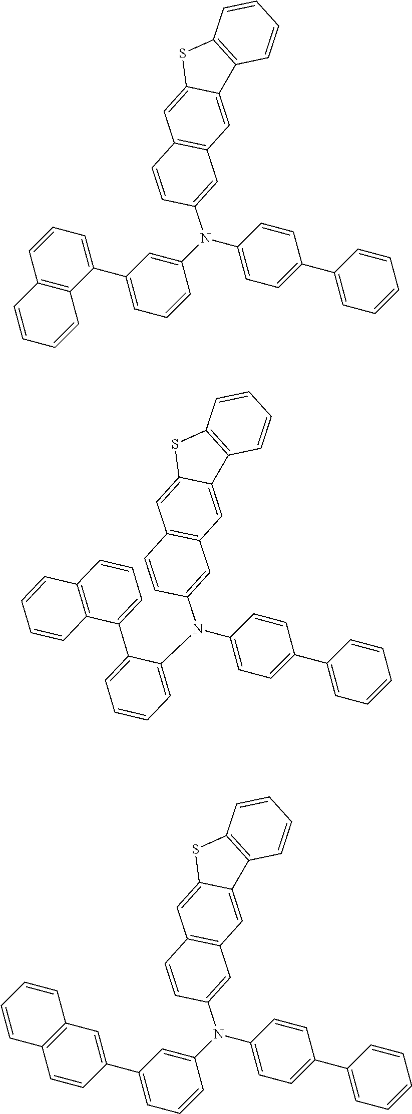 [SMILES: c1ccc(-c2ccc(N(c3cccc(-c4cccc5ccccc45)c3)c3ccc4cc5sc6ccccc6c5cc4c3)cc2)cc1 ; c1ccc(-c2ccc(N(c3ccc4cc5sc6ccccc6c5cc4c3)c3ccccc3-c3cccc4ccccc34)cc2)cc1 ; c1ccc(-c2ccc(N(c3cccc(-c4ccc5ccccc5c4)c3)c3ccc4cc5sc6ccccc6c5cc4c3)cc2)cc1] 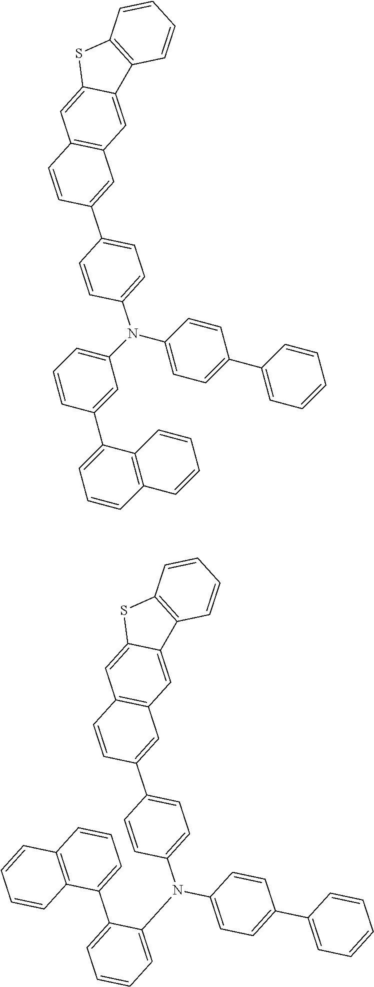 [SMILES: c1ccc(-c2ccc(N(c3ccc(-c4ccc5cc6sc7ccccc7c6cc5c4)cc3)c3cccc(-c4cccc5ccccc45)c3)cc2)cc1 ; c1ccc(-c2ccc(N(c3ccc(-c4ccc5cc6sc7ccccc7c6cc5c4)cc3)c3ccccc3-c3cccc4ccccc34)cc2)cc1] 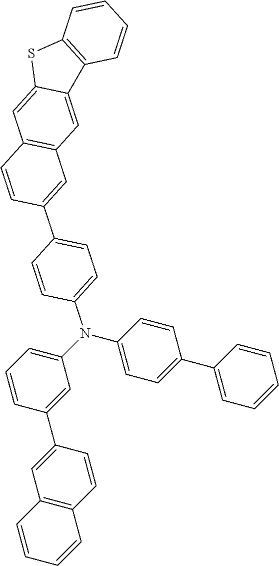 [SMILES: c1ccc(-c2ccc(N(c3ccc(-c4ccc5cc6sc7ccccc7c6cc5c4)cc3)c3cccc(-c4ccc5ccccc5c4)c3)cc2)cc1] 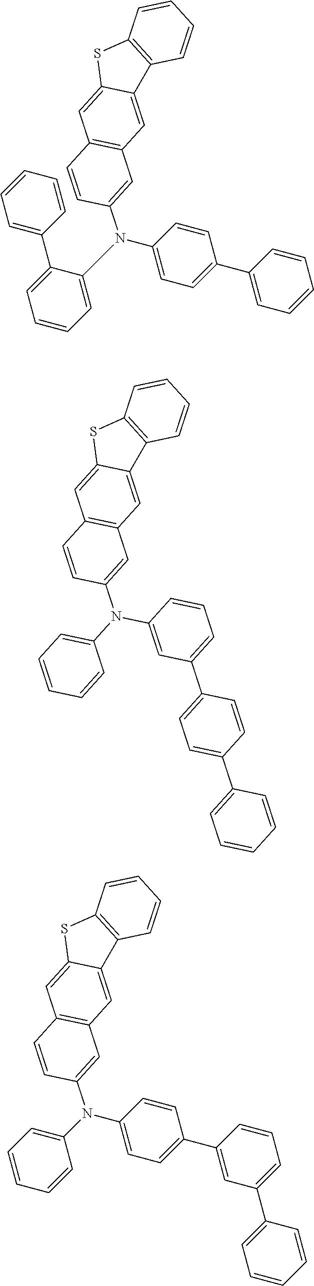 [SMILES: c1ccc(-c2ccc(N(c3ccc4cc5sc6ccccc6c5cc4c3)c3ccccc3-c3ccccc3)cc2)cc1 ; c1ccc(-c2ccc(-c3cccc(N(c4ccccc4)c4ccc5cc6sc7ccccc7c6cc5c4)c3)cc2)cc1 ; c1ccc(-c2cccc(-c3ccc(N(c4ccccc4)c4ccc5cc6sc7ccccc7c6cc5c4)cc3)c2)cc1] 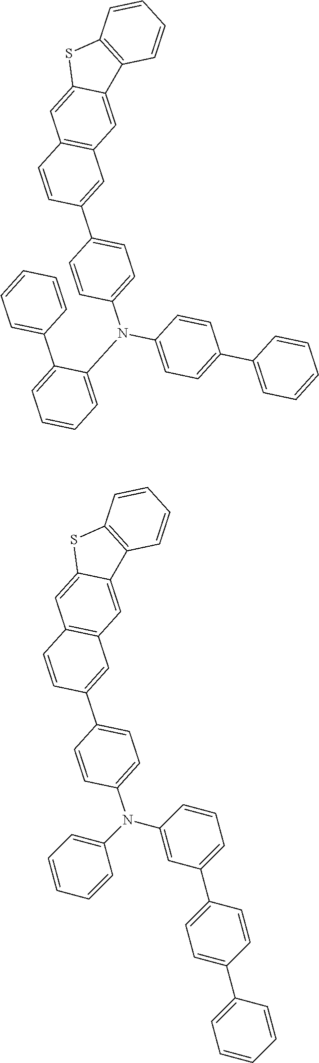 [SMILES: c1ccc(-c2ccc(N(c3ccc(-c4ccc5cc6sc7ccccc7c6cc5c4)cc3)c3ccccc3-c3ccccc3)cc2)cc1 ; c1ccc(-c2ccc(-c3cccc(N(c4ccccc4)c4ccc(-c5ccc6cc7sc8ccccc8c7cc6c5)cc4)c3)cc2)cc1] 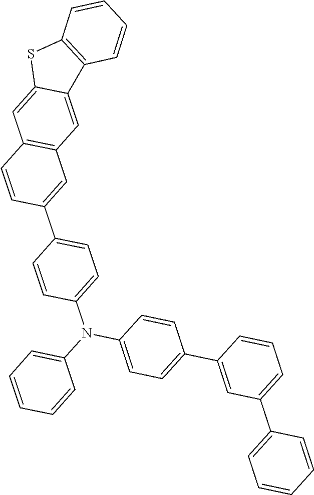 [SMILES: c1ccc(-c2cccc(-c3ccc(N(c4ccccc4)c4ccc(-c5ccc6cc7sc8ccccc8c7cc6c5)cc4)cc3)c2)cc1] 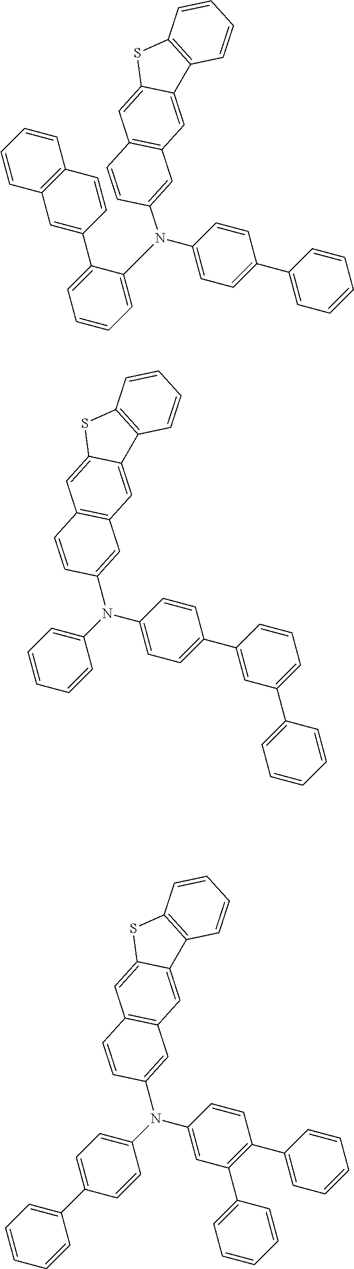 [SMILES: c1ccc(-c2ccc(N(c3ccc4cc5sc6ccccc6c5cc4c3)c3ccccc3-c3ccc4ccccc4c3)cc2)cc1 ; c1ccc(-c2cccc(-c3ccc(N(c4ccccc4)c4ccc5cc6sc7ccccc7c6cc5c4)cc3)c2)cc1 ; c1ccc(-c2ccc(N(c3ccc(-c4ccccc4)c(-c4ccccc4)c3)c3ccc4cc5sc6ccccc6c5cc4c3)cc2)cc1] 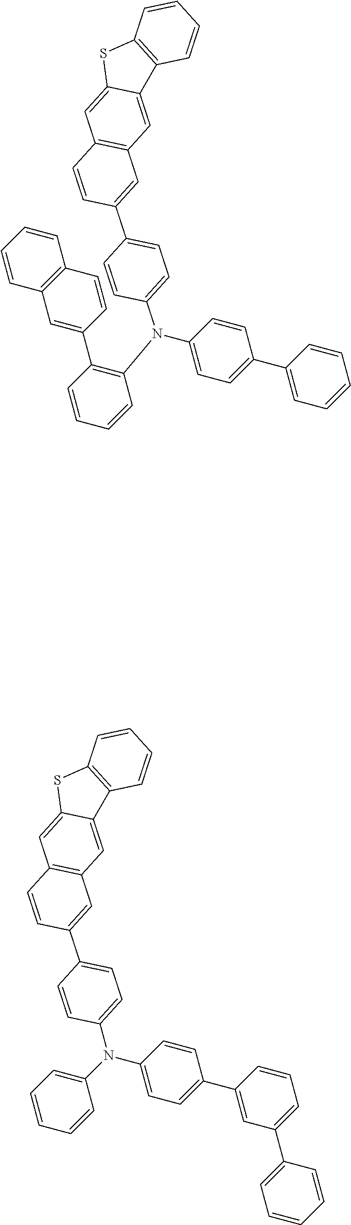 [SMILES: c1ccc(-c2ccc(N(c3ccc(-c4ccc5cc6sc7ccccc7c6cc5c4)cc3)c3ccccc3-c3ccc4ccccc4c3)cc2)cc1 ; c1ccc(-c2cccc(-c3ccc(N(c4ccccc4)c4ccc(-c5ccc6cc7sc8ccccc8c7cc6c5)cc4)cc3)c2)cc1] 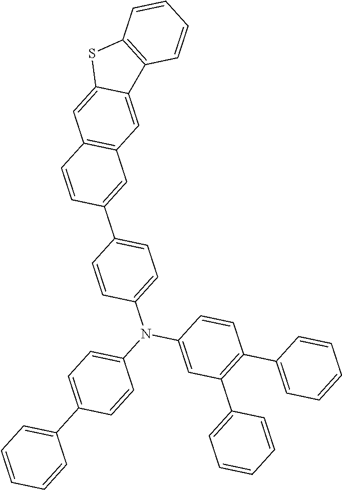 [SMILES: c1ccc(-c2ccc(N(c3ccc(-c4ccc5cc6sc7ccccc7c6cc5c4)cc3)c3ccc(-c4ccccc4)c(-c4ccccc4)c3)cc2)cc1] 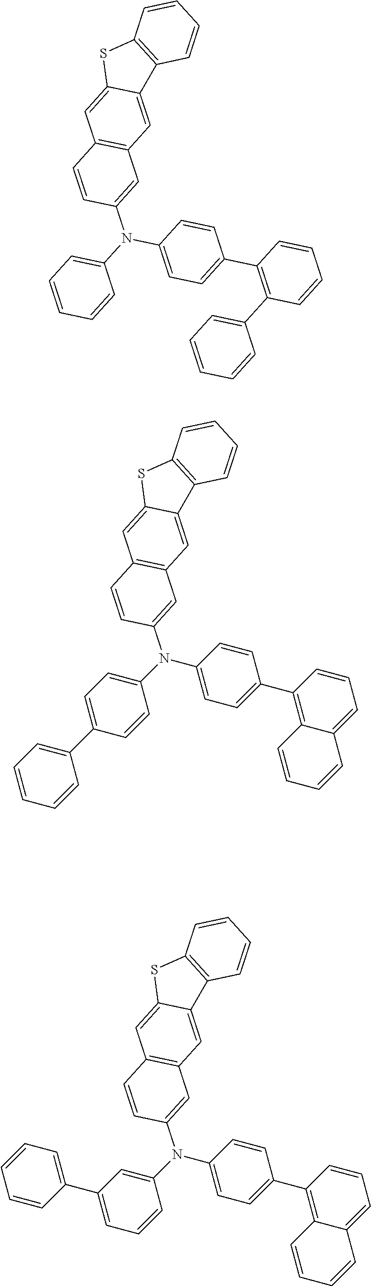 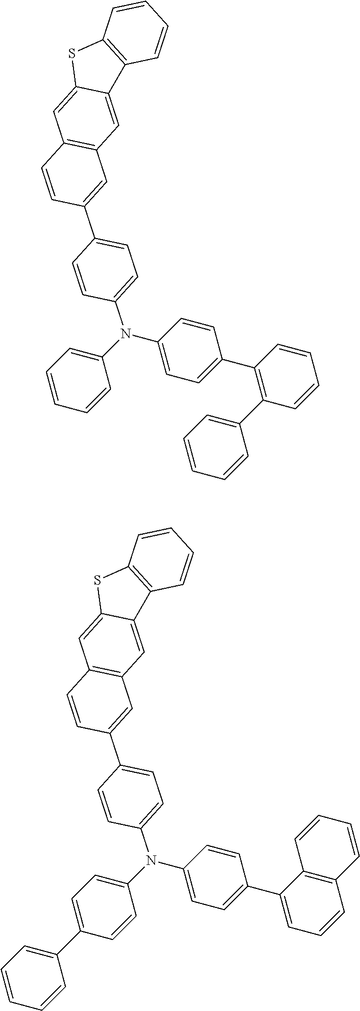 [SMILES: c1ccc(-c2ccccc2-c2ccc(N(c3ccccc3)c3ccc(-c4ccc5cc6sc7ccccc7c6cc5c4)cc3)cc2)cc1 ; c1ccc(-c2ccc(N(c3ccc(-c4ccc5cc6sc7ccccc7c6cc5c4)cc3)c3ccc(-c4cccc5ccccc45)cc3)cc2)cc1] 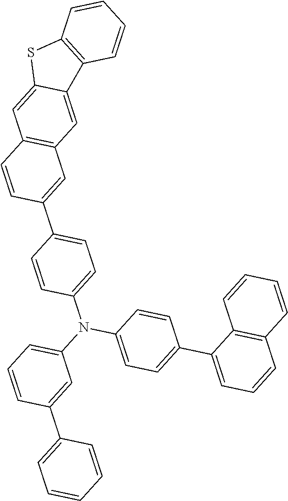 [SMILES: c1ccc(-c2cccc(N(c3ccc(-c4ccc5cc6sc7ccccc7c6cc5c4)cc3)c3ccc(-c4cccc5ccccc45)cc3)c2)cc1] 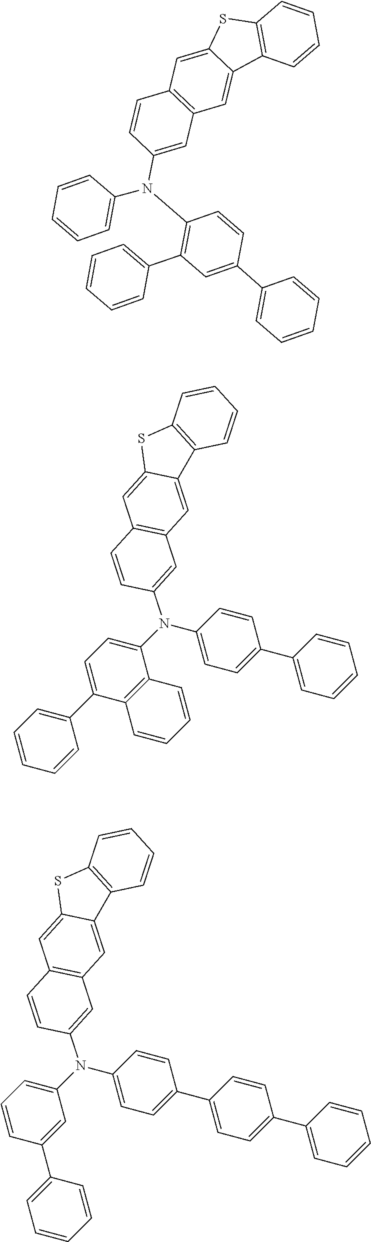 [SMILES: c1ccc(-c2ccc(N(c3ccccc3)c3ccc4cc5sc6ccccc6c5cc4c3)c(-c3ccccc3)c2)cc1 ; c1ccc(-c2ccc(N(c3ccc4cc5sc6ccccc6c5cc4c3)c3ccc(-c4ccccc4)c4ccccc34)cc2)cc1 ; c1ccc(-c2ccc(-c3ccc(N(c4cccc(-c5ccccc5)c4)c4ccc5cc6sc7ccccc7c6cc5c4)cc3)cc2)cc1] 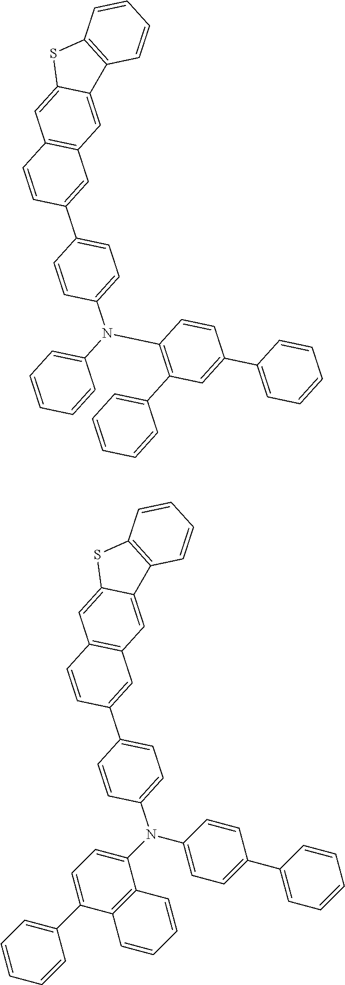 [SMILES: c1ccc(-c2ccc(N(c3ccccc3)c3ccc(-c4ccc5cc6sc7ccccc7c6cc5c4)cc3)c(-c3ccccc3)c2)cc1 ; c1ccc(-c2ccc(N(c3ccc(-c4ccc5cc6sc7ccccc7c6cc5c4)cc3)c3ccc(-c4ccccc4)c4ccccc34)cc2)cc1] 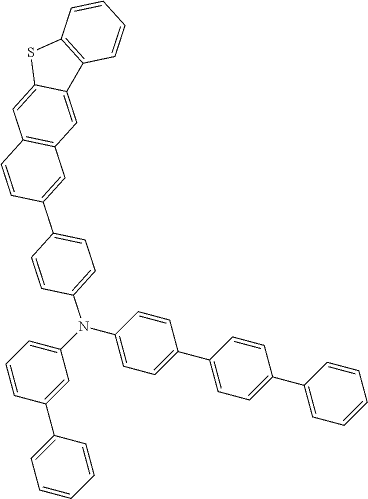 [SMILES: c1ccc(-c2ccc(-c3ccc(N(c4ccc(-c5ccc6cc7sc8ccccc8c7cc6c5)cc4)c4cccc(-c5ccccc5)c4)cc3)cc2)cc1] 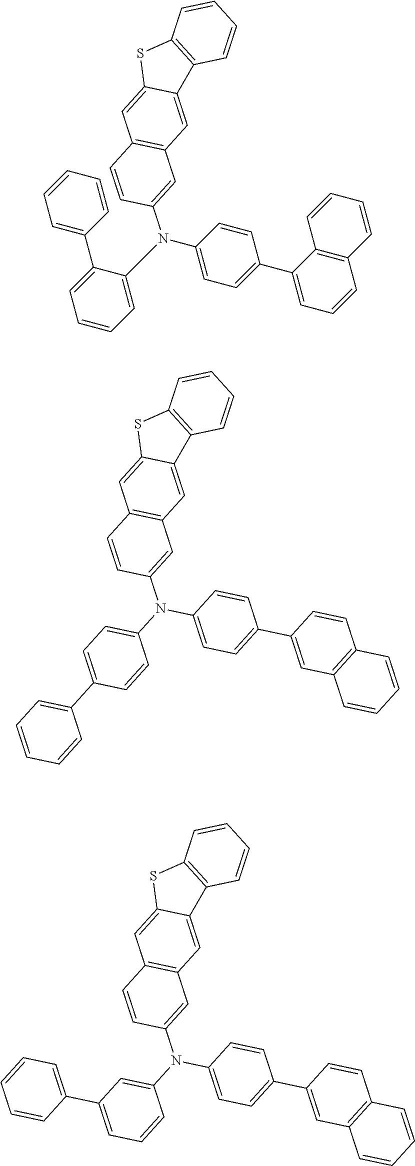 [SMILES: c1ccc(-c2ccccc2N(c2ccc(-c3cccc4ccccc34)cc2)c2ccc3cc4sc5ccccc5c4cc3c2)cc1 ; c1ccc(-c2ccc(N(c3ccc(-c4ccc5ccccc5c4)cc3)c3ccc4cc5sc6ccccc6c5cc4c3)cc2)cc1 ; c1ccc(-c2cccc(N(c3ccc(-c4ccc5ccccc5c4)cc3)c3ccc4cc5sc6ccccc6c5cc4c3)c2)cc1] 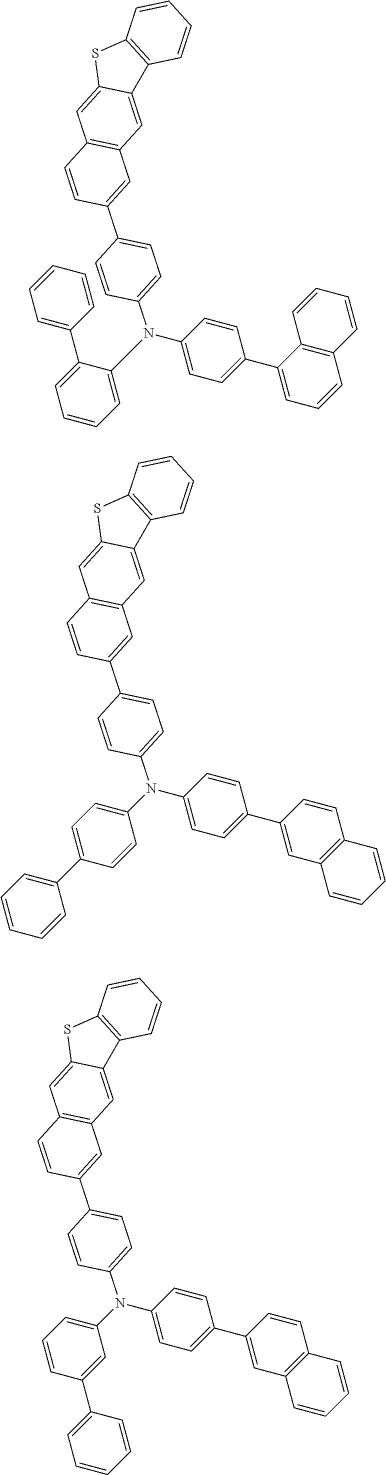 [SMILES: c1ccc(-c2ccccc2N(c2ccc(-c3ccc4cc5sc6ccccc6c5cc4c3)cc2)c2ccc(-c3cccc4ccccc34)cc2)cc1 ; c1ccc(-c2ccc(N(c3ccc(-c4ccc5ccccc5c4)cc3)c3ccc(-c4ccc5cc6sc7ccccc7c6cc5c4)cc3)cc2)cc1 ; c1ccc(-c2cccc(N(c3ccc(-c4ccc5ccccc5c4)cc3)c3ccc(-c4ccc5cc6sc7ccccc7c6cc5c4)cc3)c2)cc1] 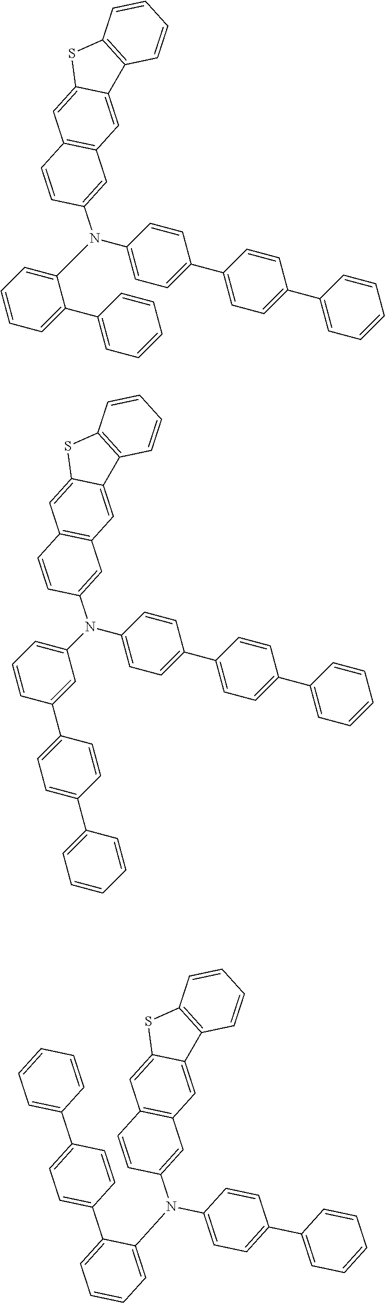 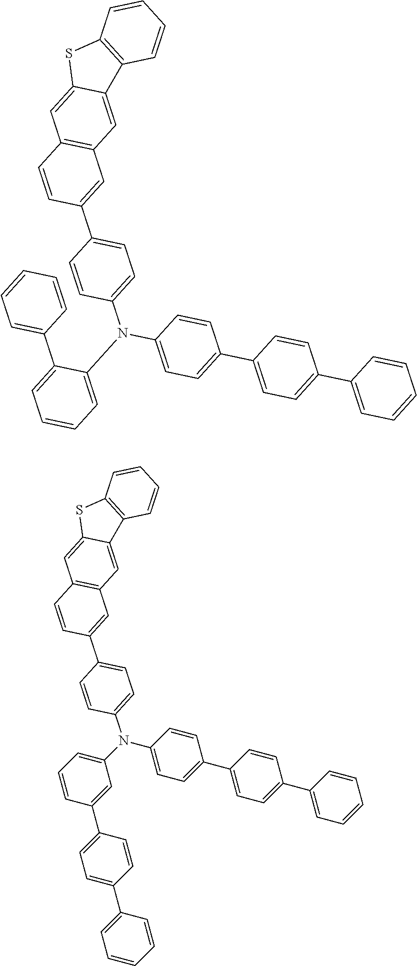 [SMILES: c1ccc(-c2ccc(-c3ccc(N(c4ccc(-c5ccc6cc7sc8ccccc8c7cc6c5)cc4)c4ccccc4-c4ccccc4)cc3)cc2)cc1 ; c1ccc(-c2ccc(-c3ccc(N(c4ccc(-c5ccc6cc7sc8ccccc8c7cc6c5)cc4)c4cccc(-c5ccc(-c6ccccc6)cc5)c4)cc3)cc2)cc1] 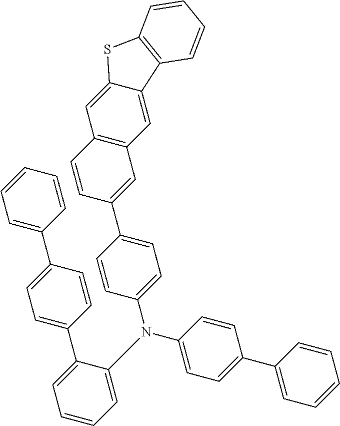 [SMILES: c1ccc(-c2ccc(-c3ccccc3N(c3ccc(-c4ccccc4)cc3)c3ccc(-c4ccc5cc6sc7ccccc7c6cc5c4)cc3)cc2)cc1] 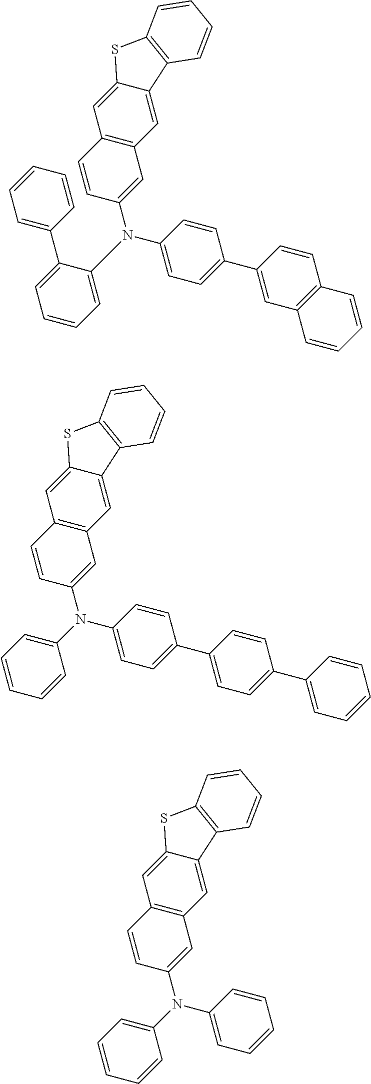 [SMILES: c1ccc(-c2ccccc2N(c2ccc(-c3ccc4ccccc4c3)cc2)c2ccc3cc4sc5ccccc5c4cc3c2)cc1 ; c1ccc(-c2ccc(-c3ccc(N(c4ccccc4)c4ccc5cc6sc7ccccc7c6cc5c4)cc3)cc2)cc1 ; c1ccc(N(c2ccccc2)c2ccc3cc4sc5ccccc5c4cc3c2)cc1] 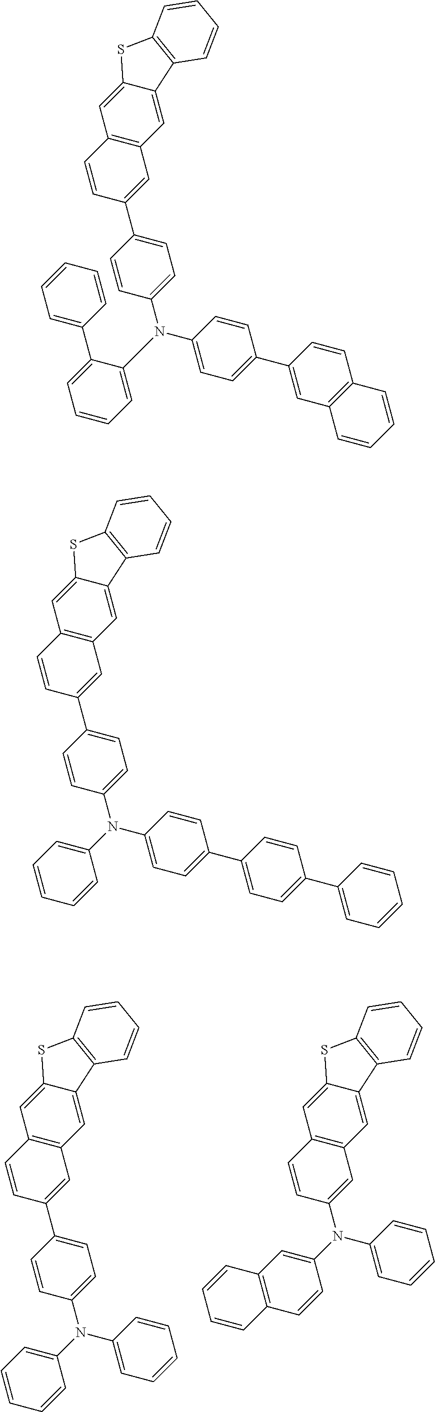 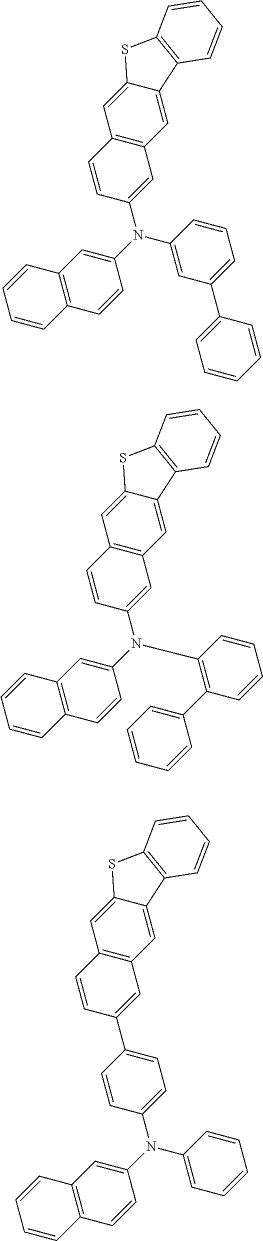 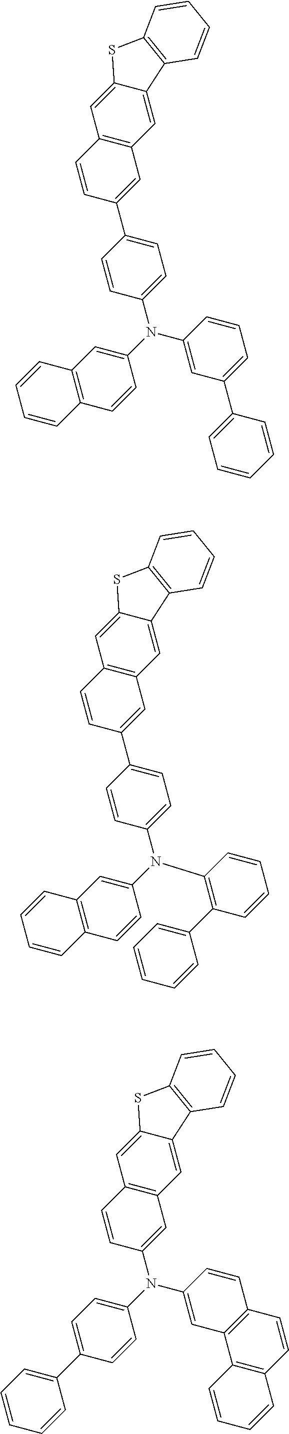 [SMILES: c1ccc(-c2cccc(N(c3ccc(-c4ccc5cc6sc7ccccc7c6cc5c4)cc3)c3ccc4ccccc4c3)c2)cc1 ; c1ccc(-c2ccccc2N(c2ccc(-c3ccc4cc5sc6ccccc6c5cc4c3)cc2)c2ccc3ccccc3c2)cc1 ; c1ccc(-c2ccc(N(c3ccc4cc5sc6ccccc6c5cc4c3)c3ccc4ccc5ccccc5c4c3)cc2)cc1] 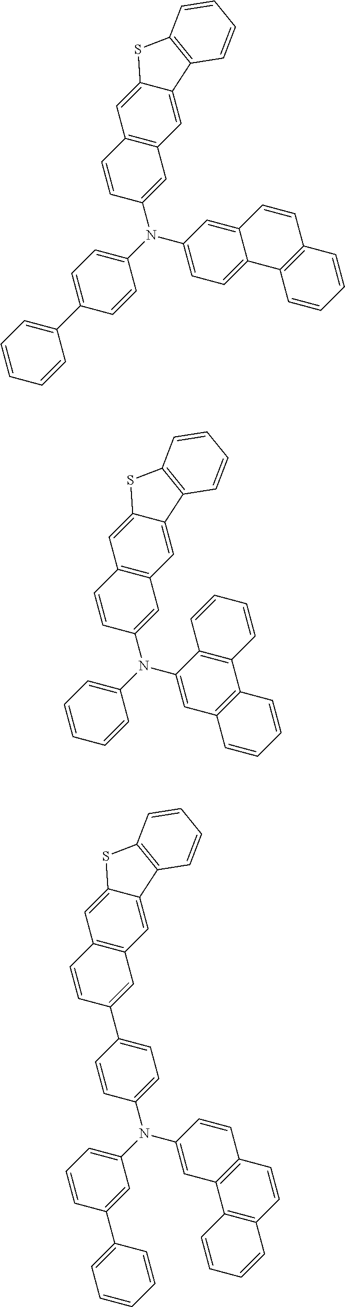 [SMILES: c1ccc(-c2ccc(N(c3ccc4cc5sc6ccccc6c5cc4c3)c3ccc4c(ccc5ccccc54)c3)cc2)cc1 ; c1ccc(N(c2ccc3cc4sc5ccccc5c4cc3c2)c2cc3ccccc3c3ccccc23)cc1 ; c1ccc(-c2cccc(N(c3ccc(-c4ccc5cc6sc7ccccc7c6cc5c4)cc3)c3ccc4ccc5ccccc5c4c3)c2)cc1] 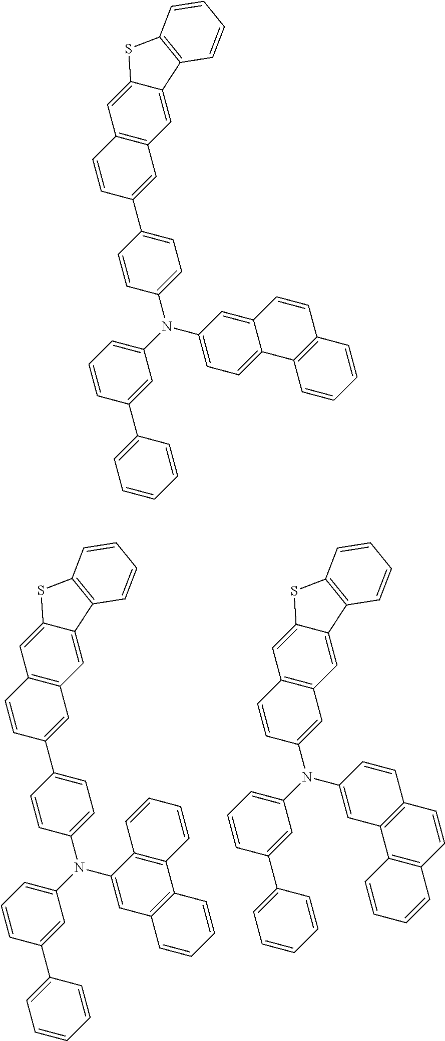 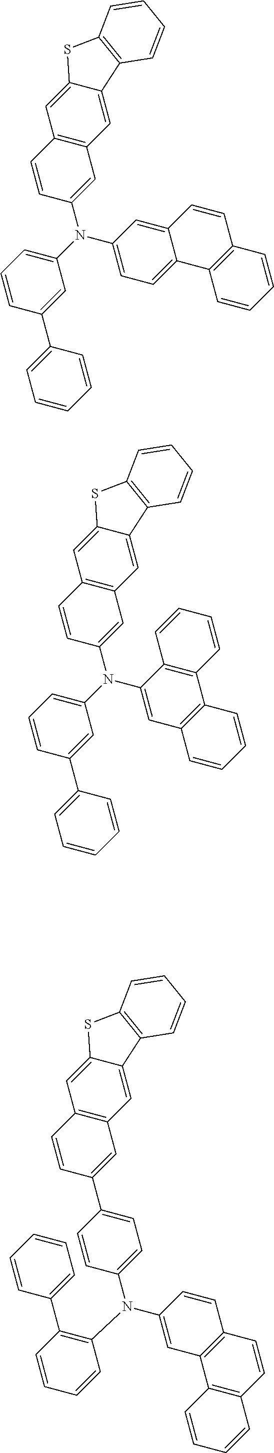 [SMILES: c1ccc(-c2cccc(N(c3ccc4cc5sc6ccccc6c5cc4c3)c3ccc4c(ccc5ccccc54)c3)c2)cc1 ; c1ccc(-c2cccc(N(c3ccc4cc5sc6ccccc6c5cc4c3)c3cc4ccccc4c4ccccc34)c2)cc1 ; c1ccc(-c2ccccc2N(c2ccc(-c3ccc4cc5sc6ccccc6c5cc4c3)cc2)c2ccc3ccc4ccccc4c3c2)cc1] 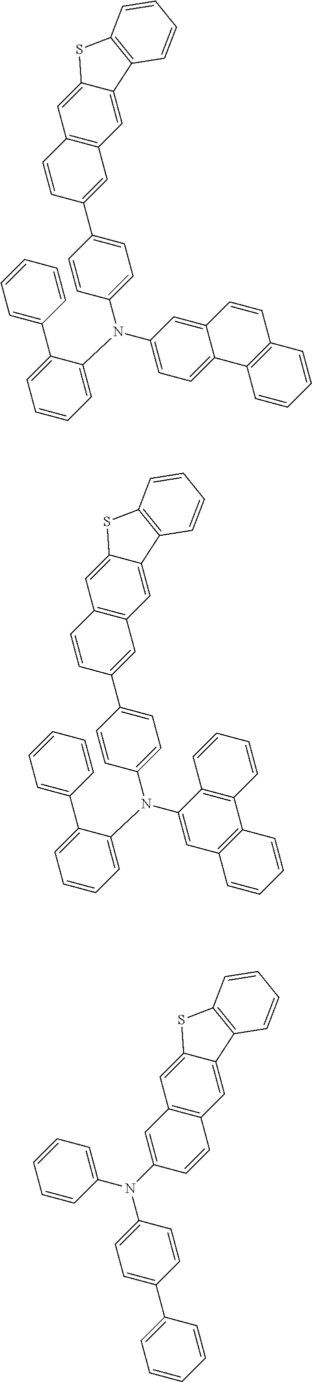 [SMILES: c1ccc(-c2ccccc2N(c2ccc(-c3ccc4cc5sc6ccccc6c5cc4c3)cc2)c2ccc3c(ccc4ccccc43)c2)cc1 ; c1ccc(-c2ccccc2N(c2ccc(-c3ccc4cc5sc6ccccc6c5cc4c3)cc2)c2cc3ccccc3c3ccccc23)cc1 ; c1ccc(-c2ccc(N(c3ccccc3)c3ccc4cc5c(cc4c3)sc3ccccc35)cc2)cc1] 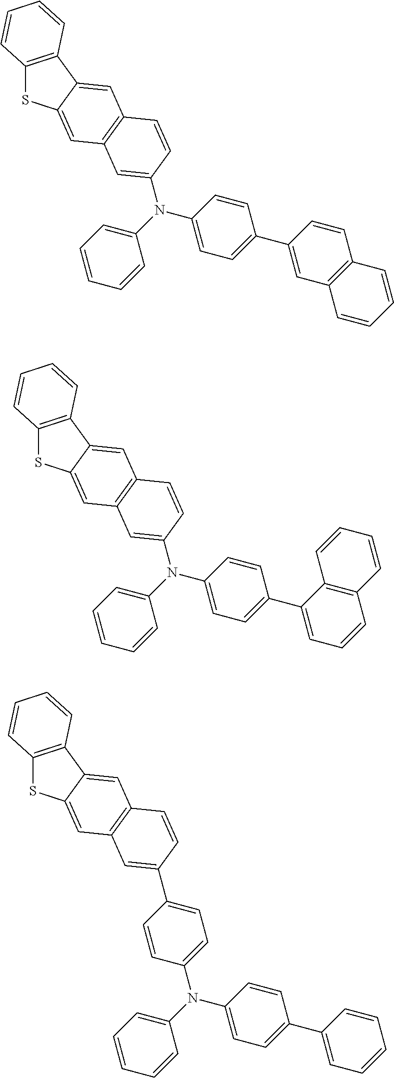 [SMILES: c1ccc(N(c2ccc(-c3ccc4ccccc4c3)cc2)c2ccc3cc4c(cc3c2)sc2ccccc24)cc1 ; c1ccc(N(c2ccc(-c3cccc4ccccc34)cc2)c2ccc3cc4c(cc3c2)sc2ccccc24)cc1 ; c1ccc(-c2ccc(N(c3ccccc3)c3ccc(-c4ccc5cc6c(cc5c4)sc4ccccc46)cc3)cc2)cc1] 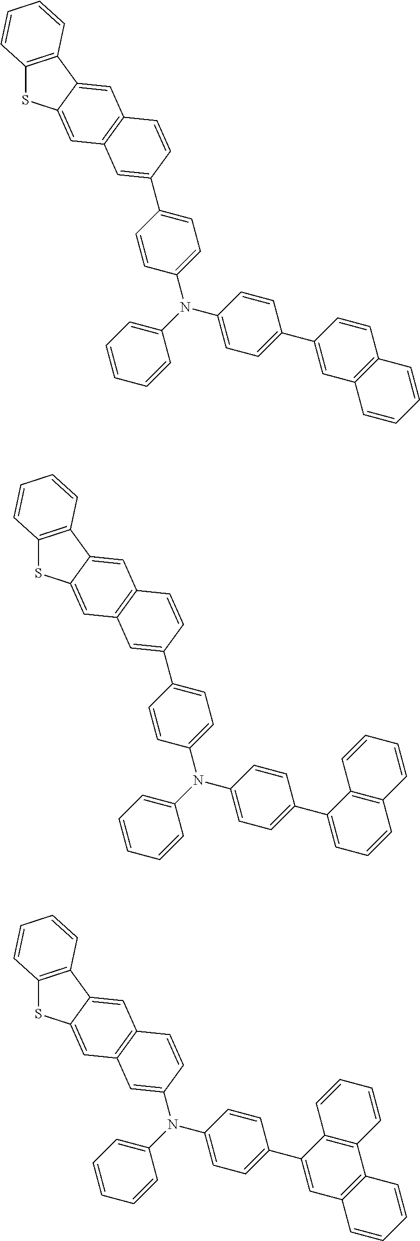 [SMILES: c1ccc(N(c2ccc(-c3ccc4ccccc4c3)cc2)c2ccc(-c3ccc4cc5c(cc4c3)sc3ccccc35)cc2)cc1 ; c1ccc(N(c2ccc(-c3ccc4cc5c(cc4c3)sc3ccccc35)cc2)c2ccc(-c3cccc4ccccc34)cc2)cc1 ; c1ccc(N(c2ccc(-c3cc4ccccc4c4ccccc34)cc2)c2ccc3cc4c(cc3c2)sc2ccccc24)cc1] 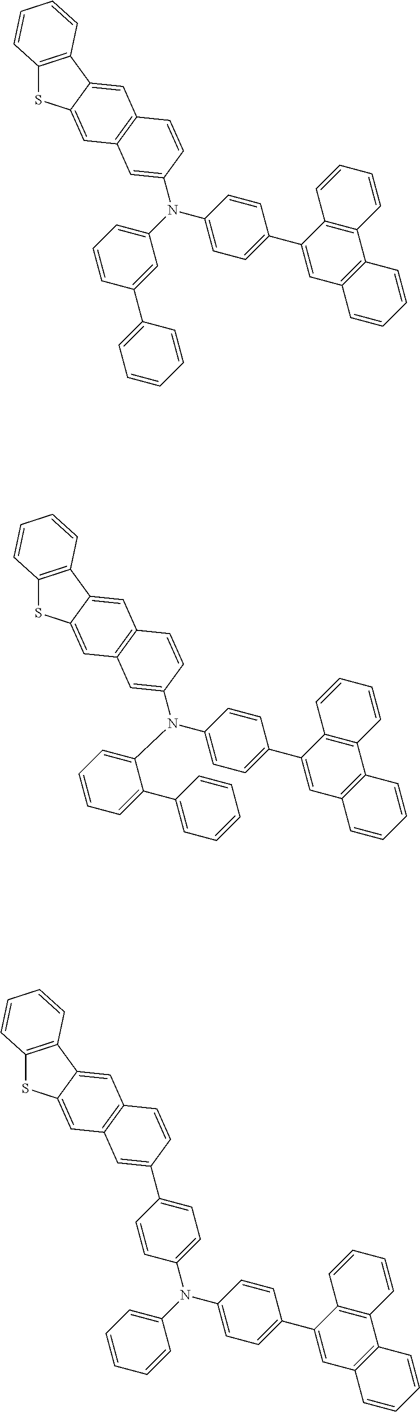 [SMILES: c1ccc(-c2cccc(N(c3ccc(-c4cc5ccccc5c5ccccc45)cc3)c3ccc4cc5c(cc4c3)sc3ccccc35)c2)cc1 ; c1ccc(-c2ccccc2N(c2ccc(-c3cc4ccccc4c4ccccc34)cc2)c2ccc3cc4c(cc3c2)sc2ccccc24)cc1 ; c1ccc(N(c2ccc(-c3ccc4cc5c(cc4c3)sc3ccccc35)cc2)c2ccc(-c3cc4ccccc4c4ccccc34)cc2)cc1] 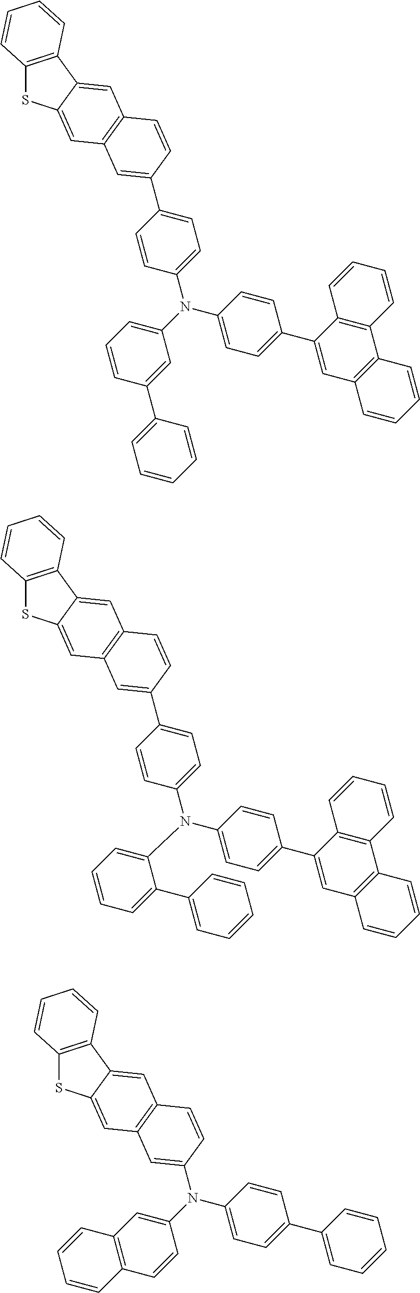 [SMILES: c1ccc(-c2cccc(N(c3ccc(-c4ccc5cc6c(cc5c4)sc4ccccc46)cc3)c3ccc(-c4cc5ccccc5c5ccccc45)cc3)c2)cc1 ; c1ccc(-c2ccccc2N(c2ccc(-c3ccc4cc5c(cc4c3)sc3ccccc35)cc2)c2ccc(-c3cc4ccccc4c4ccccc34)cc2)cc1 ; c1ccc(-c2ccc(N(c3ccc4ccccc4c3)c3ccc4cc5c(cc4c3)sc3ccccc35)cc2)cc1] 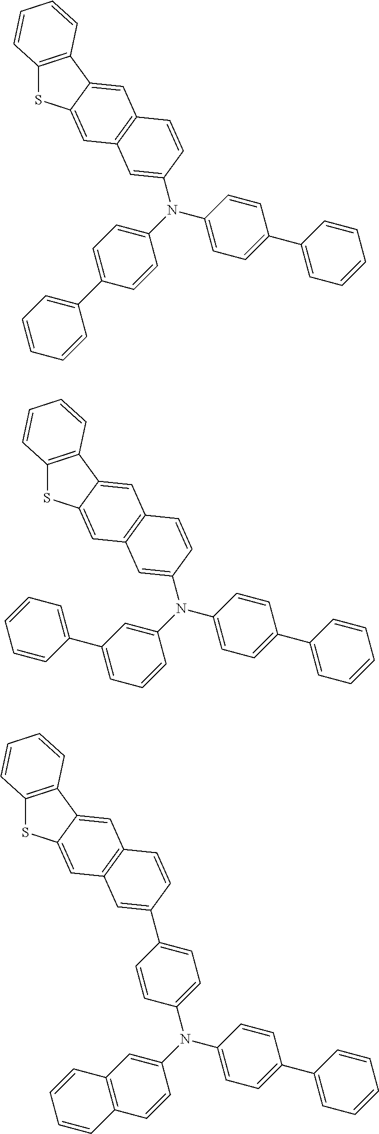 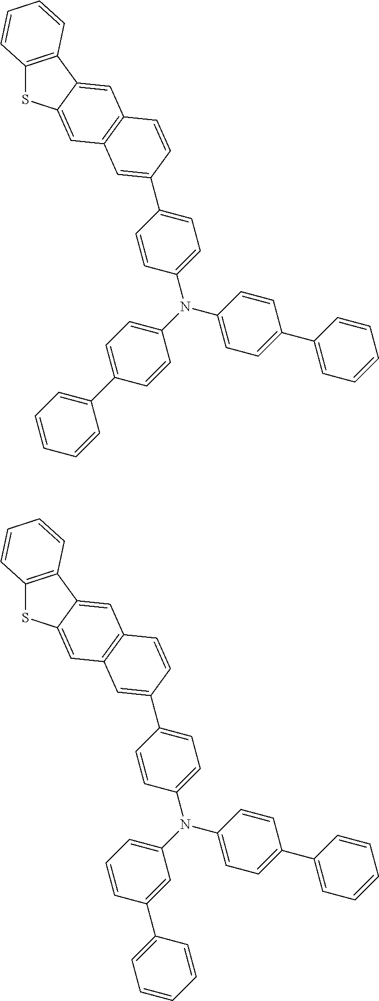 [SMILES: c1ccc(-c2ccc(N(c3ccc(-c4ccccc4)cc3)c3ccc(-c4ccc5cc6c(cc5c4)sc4ccccc46)cc3)cc2)cc1 ; c1ccc(-c2ccc(N(c3ccc(-c4ccc5cc6c(cc5c4)sc4ccccc46)cc3)c3cccc(-c4ccccc4)c3)cc2)cc1] 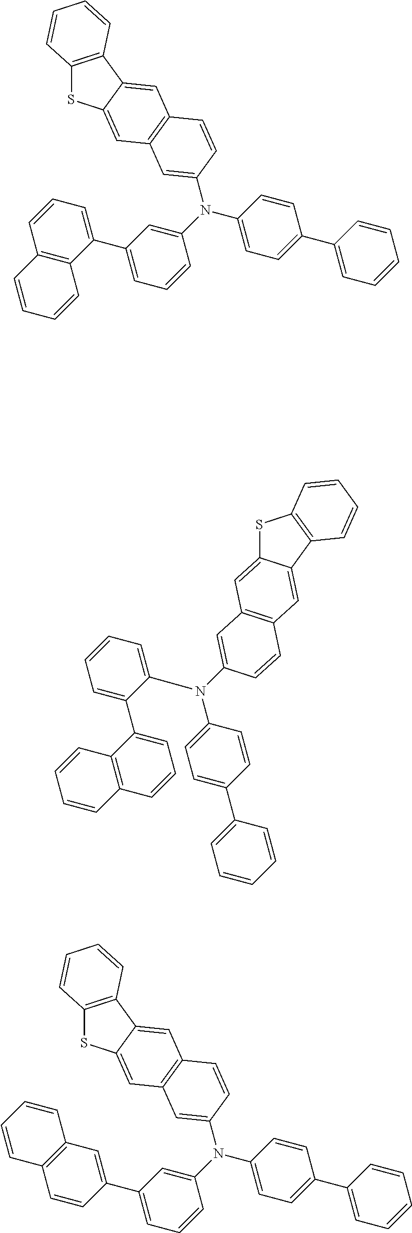 [SMILES: c1ccc(-c2ccc(N(c3cccc(-c4cccc5ccccc45)c3)c3ccc4cc5c(cc4c3)sc3ccccc35)cc2)cc1 ; c1ccc(-c2ccc(N(c3ccc4cc5c(cc4c3)sc3ccccc35)c3ccccc3-c3cccc4ccccc34)cc2)cc1 ; c1ccc(-c2ccc(N(c3cccc(-c4ccc5ccccc5c4)c3)c3ccc4cc5c(cc4c3)sc3ccccc35)cc2)cc1] 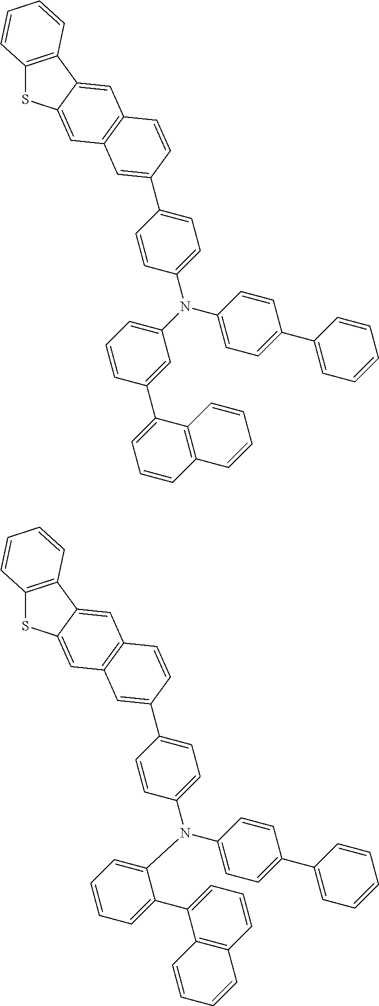 [SMILES: c1ccc(-c2ccc(N(c3ccc(-c4ccc5cc6c(cc5c4)sc4ccccc46)cc3)c3cccc(-c4cccc5ccccc45)c3)cc2)cc1 ; c1ccc(-c2ccc(N(c3ccc(-c4ccc5cc6c(cc5c4)sc4ccccc46)cc3)c3ccccc3-c3cccc4ccccc34)cc2)cc1] 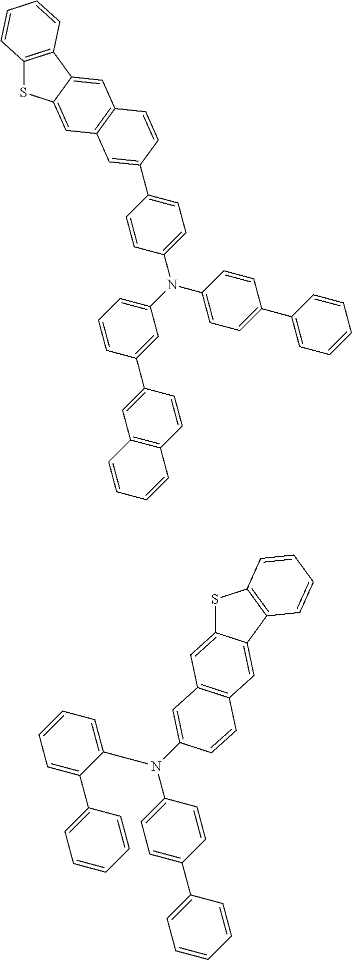 [SMILES: c1ccc(-c2ccc(N(c3ccc(-c4ccc5cc6c(cc5c4)sc4ccccc46)cc3)c3cccc(-c4ccc5ccccc5c4)c3)cc2)cc1 ; c1ccc(-c2ccc(N(c3ccc4cc5c(cc4c3)sc3ccccc35)c3ccccc3-c3ccccc3)cc2)cc1] 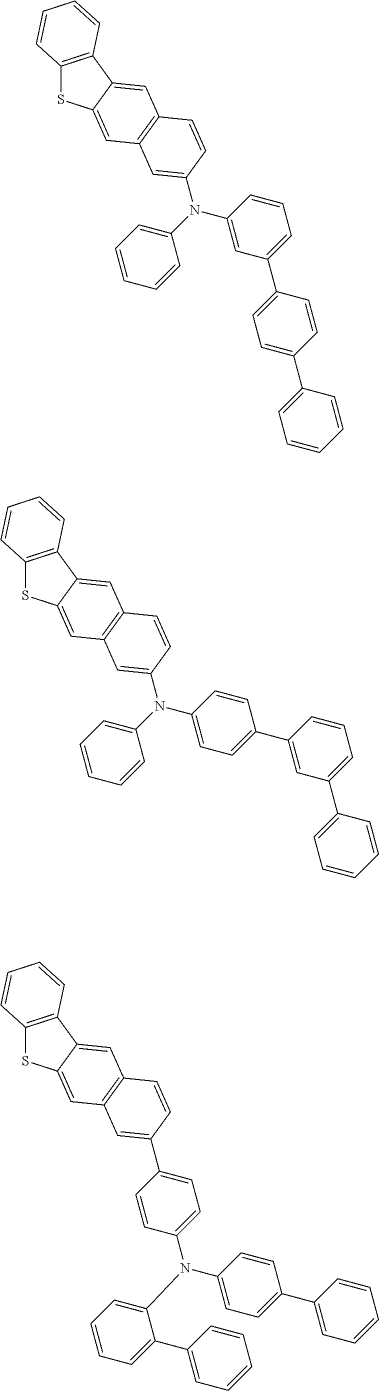 [SMILES: c1ccc(-c2ccc(-c3cccc(N(c4ccccc4)c4ccc5cc6c(cc5c4)sc4ccccc46)c3)cc2)cc1 ; c1ccc(-c2cccc(-c3ccc(N(c4ccccc4)c4ccc5cc6c(cc5c4)sc4ccccc46)cc3)c2)cc1 ; c1ccc(-c2ccc(N(c3ccc(-c4ccc5cc6c(cc5c4)sc4ccccc46)cc3)c3ccccc3-c3ccccc3)cc2)cc1] 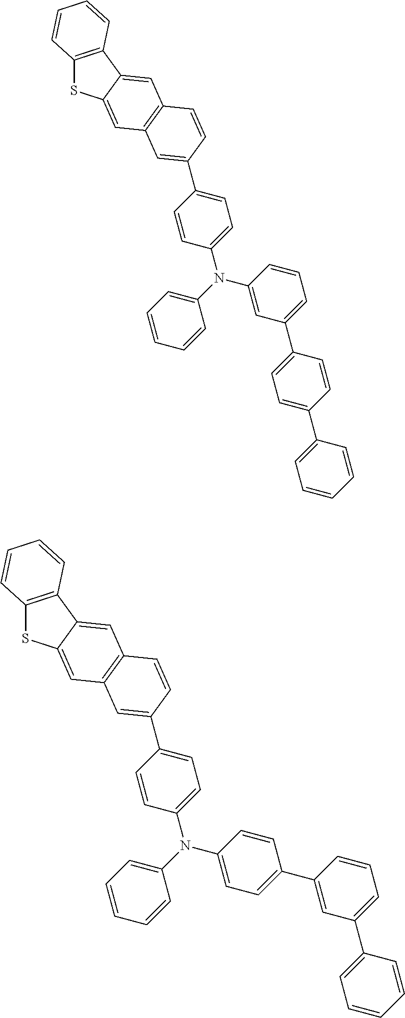 [SMILES: c1ccc(-c2ccc(-c3cccc(N(c4ccccc4)c4ccc(-c5ccc6cc7c(cc6c5)sc5ccccc57)cc4)c3)cc2)cc1 ; c1ccc(-c2cccc(-c3ccc(N(c4ccccc4)c4ccc(-c5ccc6cc7c(cc6c5)sc5ccccc57)cc4)cc3)c2)cc1] 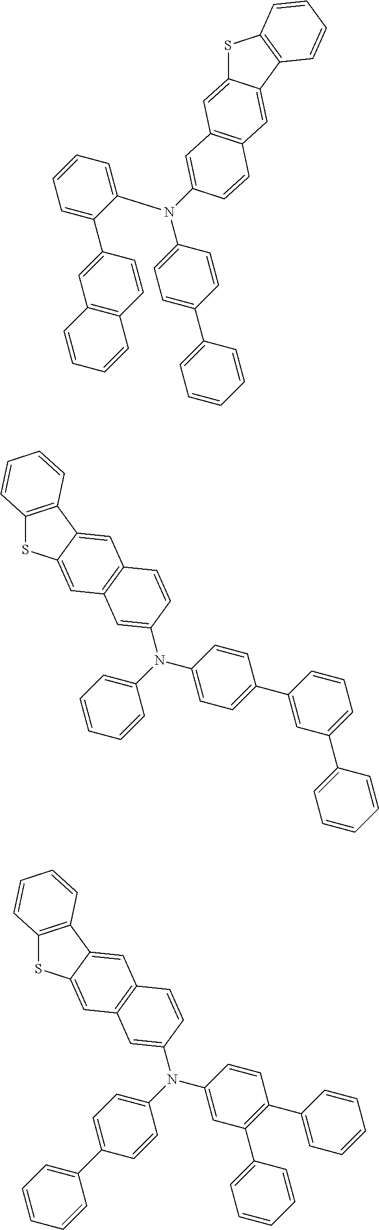 [SMILES: c1ccc(-c2ccc(N(c3ccc4cc5c(cc4c3)sc3ccccc35)c3ccccc3-c3ccc4ccccc4c3)cc2)cc1 ; c1ccc(-c2cccc(-c3ccc(N(c4ccccc4)c4ccc5cc6c(cc5c4)sc4ccccc46)cc3)c2)cc1 ; c1ccc(-c2ccc(N(c3ccc(-c4ccccc4)c(-c4ccccc4)c3)c3ccc4cc5c(cc4c3)sc3ccccc35)cc2)cc1] 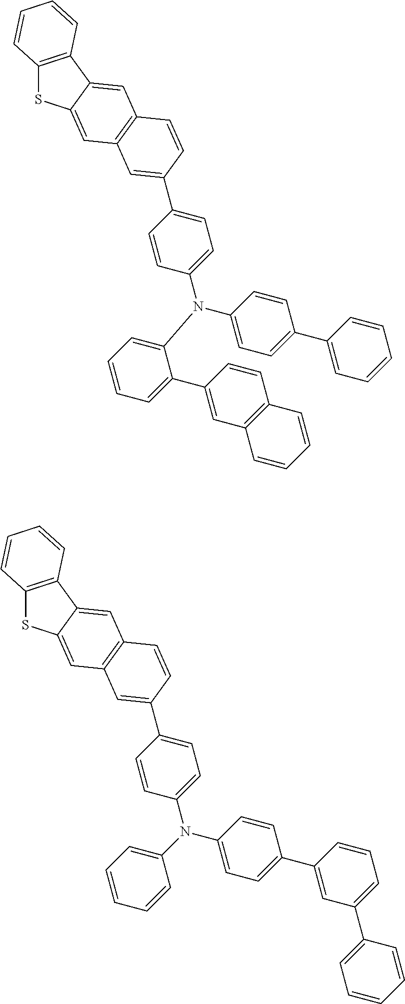 [SMILES: c1ccc(-c2ccc(N(c3ccc(-c4ccc5cc6c(cc5c4)sc4ccccc46)cc3)c3ccccc3-c3ccc4ccccc4c3)cc2)cc1 ; c1ccc(-c2cccc(-c3ccc(N(c4ccccc4)c4ccc(-c5ccc6cc7c(cc6c5)sc5ccccc57)cc4)cc3)c2)cc1] 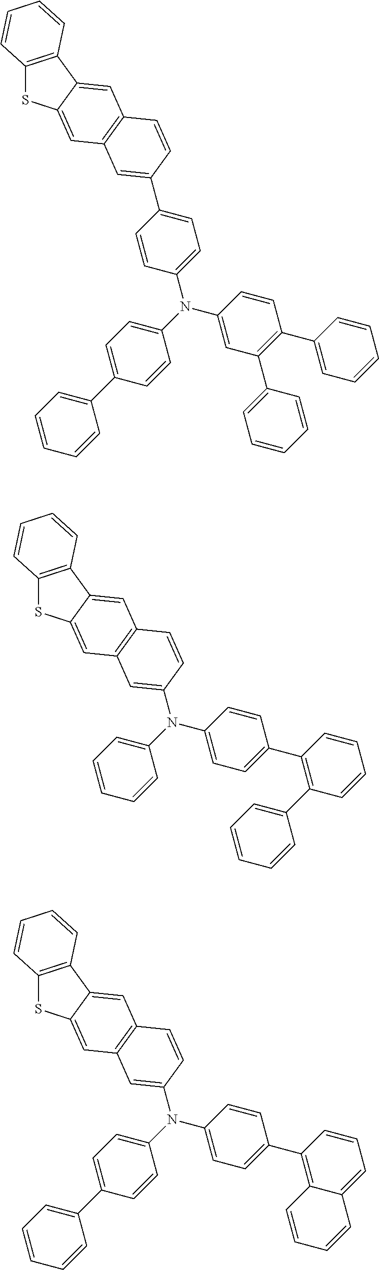 [SMILES: c1ccc(-c2ccc(N(c3ccc(-c4ccc5cc6c(cc5c4)sc4ccccc46)cc3)c3ccc(-c4ccccc4)c(-c4ccccc4)c3)cc2)cc1 ; c1ccc(-c2ccccc2-c2ccc(N(c3ccccc3)c3ccc4cc5c(cc4c3)sc3ccccc35)cc2)cc1 ; c1ccc(-c2ccc(N(c3ccc(-c4cccc5ccccc45)cc3)c3ccc4cc5c(cc4c3)sc3ccccc35)cc2)cc1] 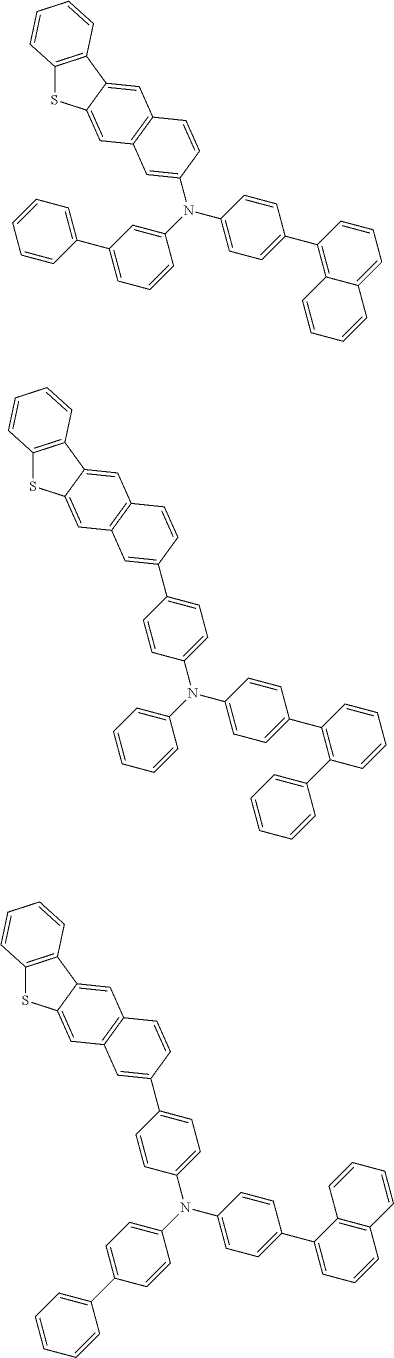 [SMILES: c1ccc(-c2cccc(N(c3ccc(-c4cccc5ccccc45)cc3)c3ccc4cc5c(cc4c3)sc3ccccc35)c2)cc1 ; c1ccc(-c2ccccc2-c2ccc(N(c3ccccc3)c3ccc(-c4ccc5cc6c(cc5c4)sc4ccccc46)cc3)cc2)cc1 ; c1ccc(-c2ccc(N(c3ccc(-c4ccc5cc6c(cc5c4)sc4ccccc46)cc3)c3ccc(-c4cccc5ccccc45)cc3)cc2)cc1] 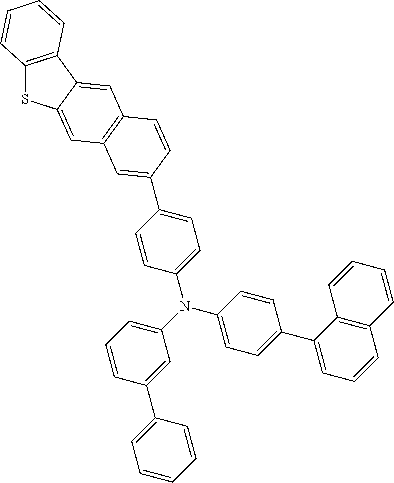 [SMILES: c1ccc(-c2cccc(N(c3ccc(-c4ccc5cc6c(cc5c4)sc4ccccc46)cc3)c3ccc(-c4cccc5ccccc45)cc3)c2)cc1] 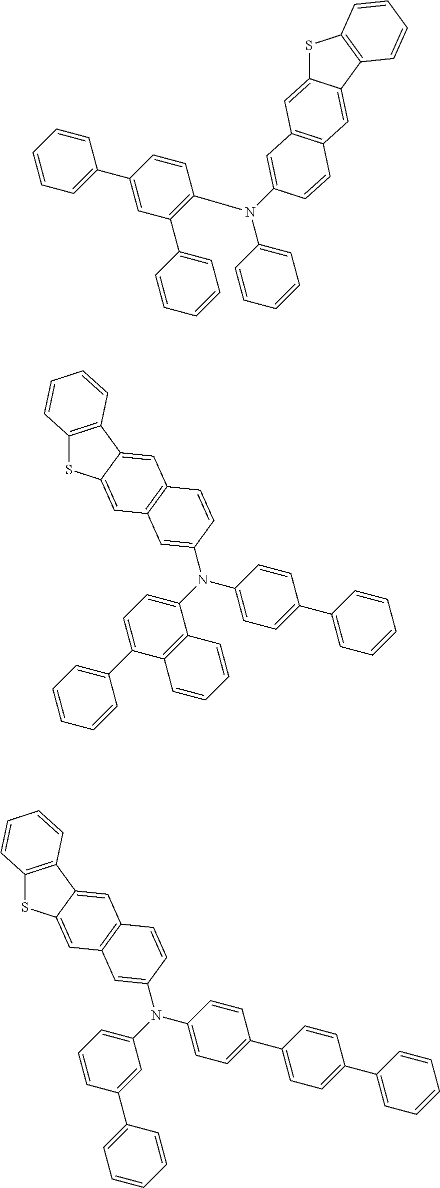 [SMILES: c1ccc(-c2ccc(N(c3ccccc3)c3ccc4cc5c(cc4c3)sc3ccccc35)c(-c3ccccc3)c2)cc1 ; c1ccc(-c2ccc(N(c3ccc4cc5c(cc4c3)sc3ccccc35)c3ccc(-c4ccccc4)c4ccccc34)cc2)cc1 ; c1ccc(-c2ccc(-c3ccc(N(c4cccc(-c5ccccc5)c4)c4ccc5cc6c(cc5c4)sc4ccccc46)cc3)cc2)cc1] 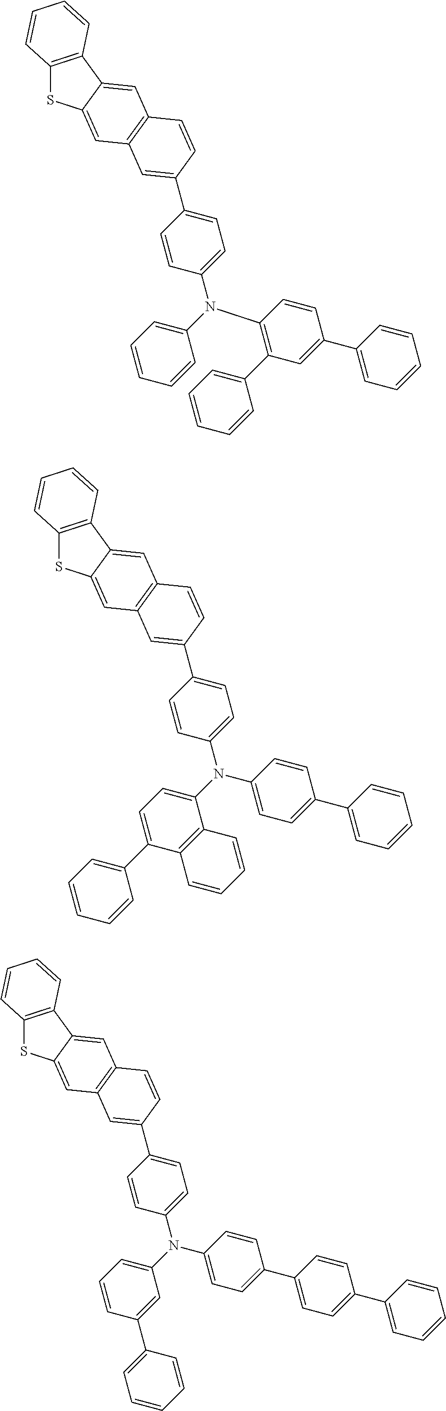 [SMILES: c1ccc(-c2ccc(N(c3ccccc3)c3ccc(-c4ccc5cc6c(cc5c4)sc4ccccc46)cc3)c(-c3ccccc3)c2)cc1 ; c1ccc(-c2ccc(N(c3ccc(-c4ccc5cc6c(cc5c4)sc4ccccc46)cc3)c3ccc(-c4ccccc4)c4ccccc34)cc2)cc1 ; c1ccc(-c2ccc(-c3ccc(N(c4ccc(-c5ccc6cc7c(cc6c5)sc5ccccc57)cc4)c4cccc(-c5ccccc5)c4)cc3)cc2)cc1] 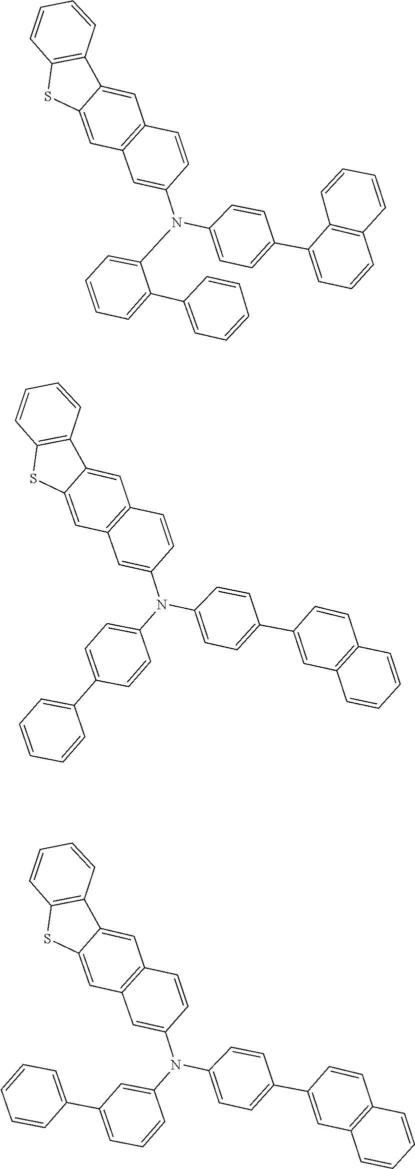 [SMILES: c1ccc(-c2ccccc2N(c2ccc(-c3cccc4ccccc34)cc2)c2ccc3cc4c(cc3c2)sc2ccccc24)cc1 ; c1ccc(-c2ccc(N(c3ccc(-c4ccc5ccccc5c4)cc3)c3ccc4cc5c(cc4c3)sc3ccccc35)cc2)cc1 ; c1ccc(-c2cccc(N(c3ccc(-c4ccc5ccccc5c4)cc3)c3ccc4cc5c(cc4c3)sc3ccccc35)c2)cc1] 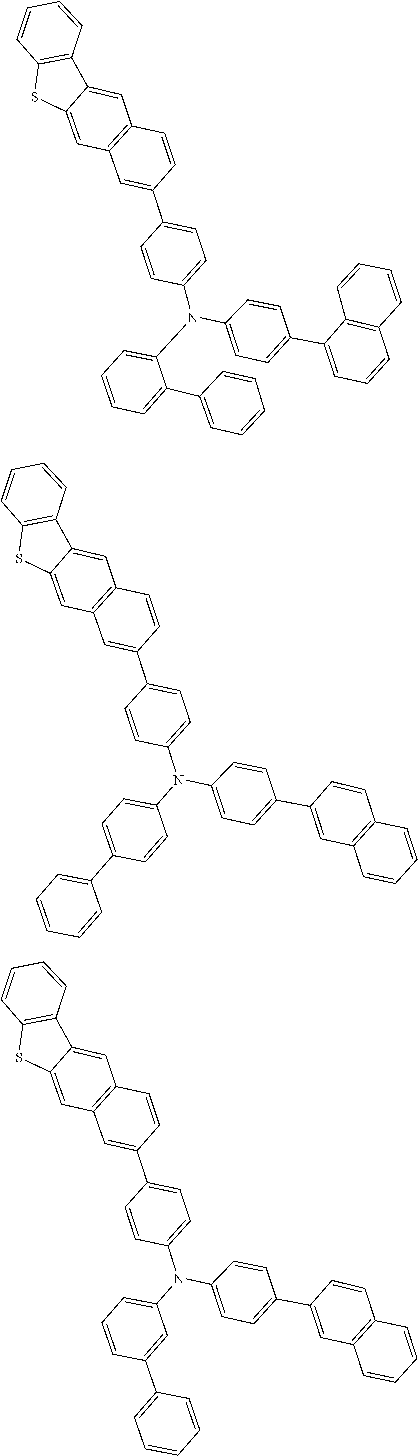 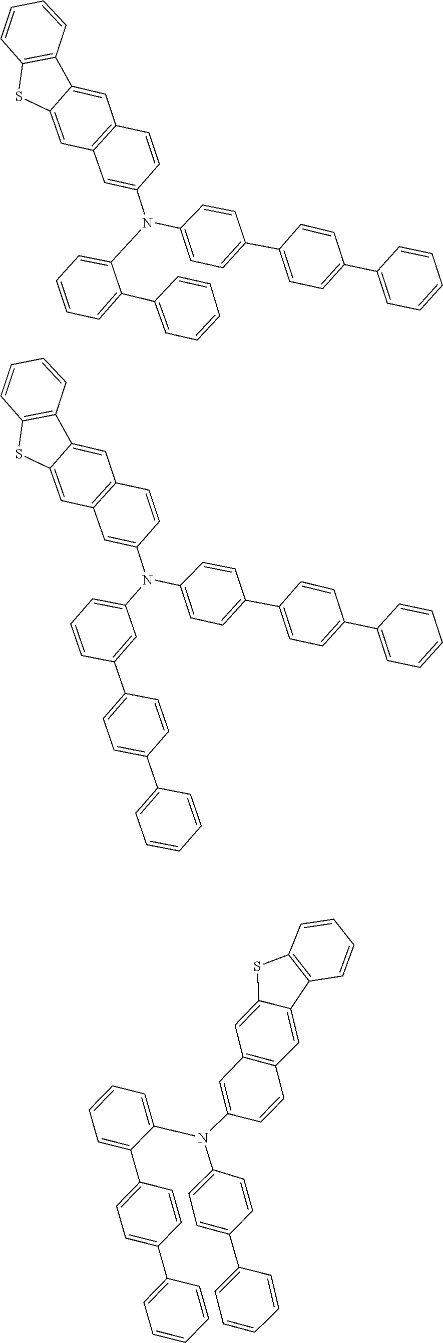 [SMILES: c1ccc(-c2ccc(-c3ccc(N(c4ccc5cc6c(cc5c4)sc4ccccc46)c4ccccc4-c4ccccc4)cc3)cc2)cc1 ; c1ccc(-c2ccc(-c3ccc(N(c4cccc(-c5ccc(-c6ccccc6)cc5)c4)c4ccc5cc6c(cc5c4)sc4ccccc46)cc3)cc2)cc1 ; c1ccc(-c2ccc(-c3ccccc3N(c3ccc(-c4ccccc4)cc3)c3ccc4cc5c(cc4c3)sc3ccccc35)cc2)cc1] 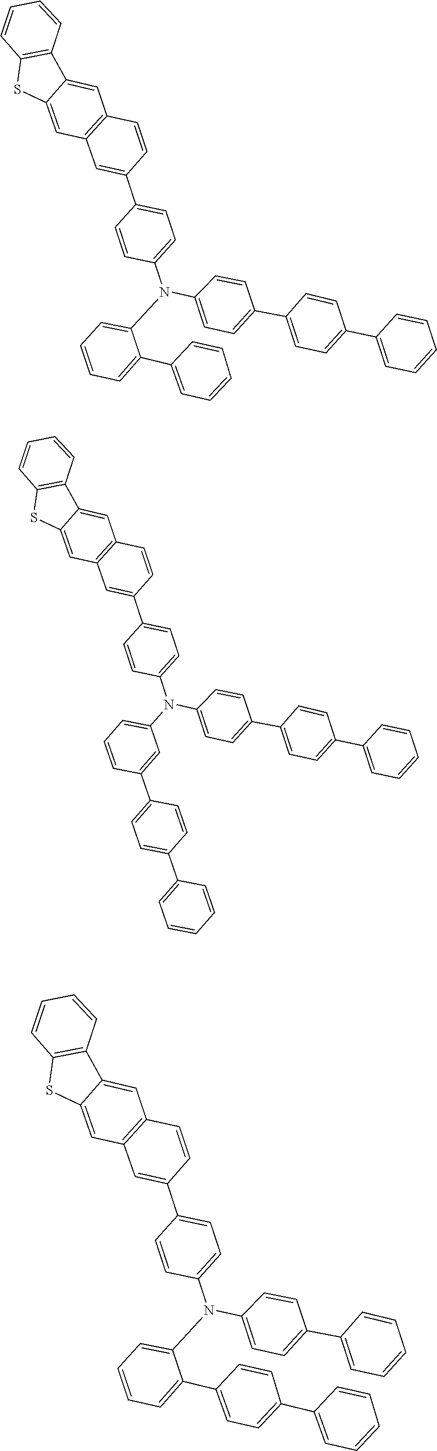 [SMILES: c1ccc(-c2ccc(-c3ccc(N(c4ccc(-c5ccc6cc7c(cc6c5)sc5ccccc57)cc4)c4ccccc4-c4ccccc4)cc3)cc2)cc1 ; c1ccc(-c2ccc(-c3ccc(N(c4ccc(-c5ccc6cc7c(cc6c5)sc5ccccc57)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5cc6c(cc5c4)sc4ccccc46)cc3)cc2)cc1] 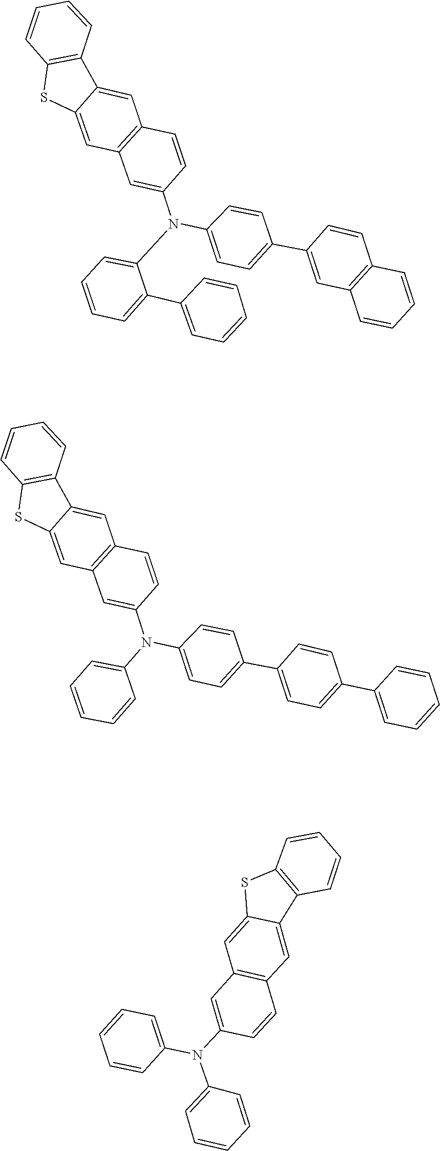 [SMILES: c1ccc(-c2ccccc2N(c2ccc(-c3ccc4ccccc4c3)cc2)c2ccc3cc4c(cc3c2)sc2ccccc24)cc1 ; c1ccc(-c2ccc(-c3ccc(N(c4ccccc4)c4ccc5cc6c(cc5c4)sc4ccccc46)cc3)cc2)cc1 ; c1ccc(N(c2ccccc2)c2ccc3cc4c(cc3c2)sc2ccccc24)cc1] 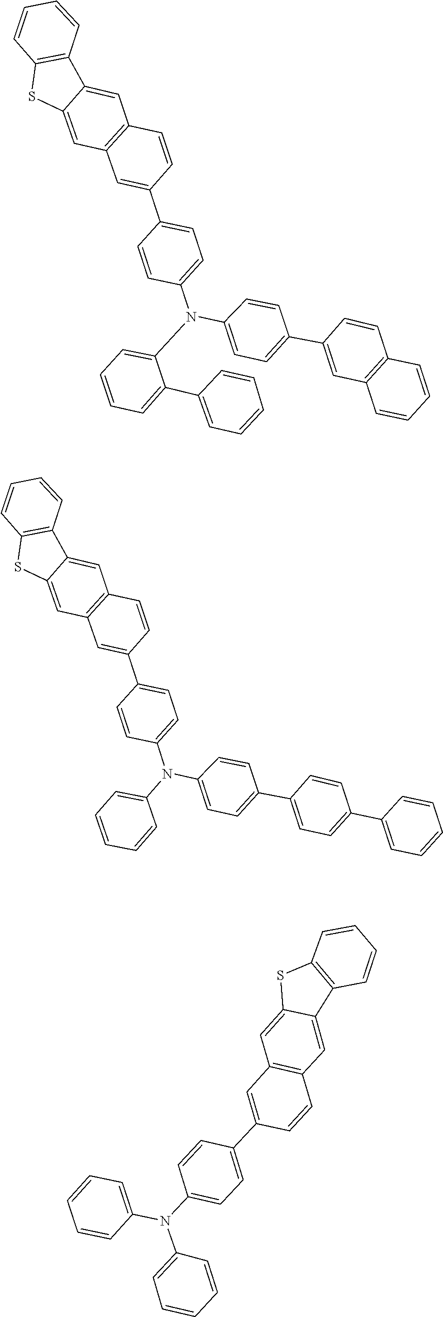 [SMILES: c1ccc(-c2ccccc2N(c2ccc(-c3ccc4ccccc4c3)cc2)c2ccc(-c3ccc4cc5c(cc4c3)sc3ccccc35)cc2)cc1 ; c1ccc(-c2ccc(-c3ccc(N(c4ccccc4)c4ccc(-c5ccc6cc7c(cc6c5)sc5ccccc57)cc4)cc3)cc2)cc1 ; c1ccc(N(c2ccccc2)c2ccc(-c3ccc4cc5c(cc4c3)sc3ccccc35)cc2)cc1] 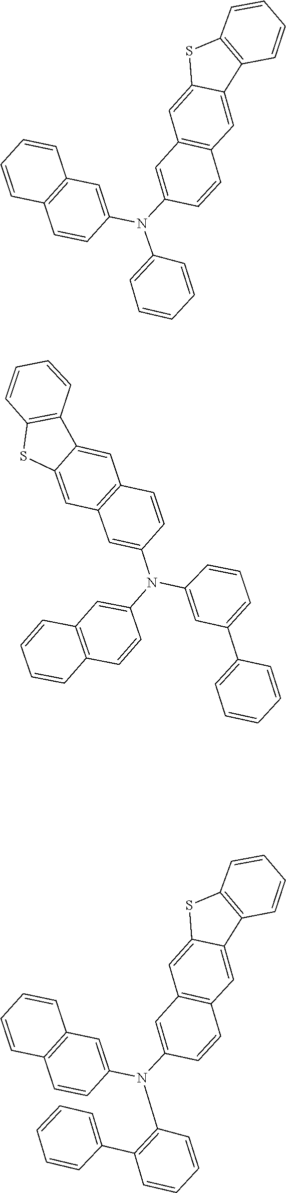 [SMILES: c1ccc(N(c2ccc3ccccc3c2)c2ccc3cc4c(cc3c2)sc2ccccc24)cc1 ; c1ccc(-c2cccc(N(c3ccc4ccccc4c3)c3ccc4cc5c(cc4c3)sc3ccccc35)c2)cc1 ; c1ccc(-c2ccccc2N(c2ccc3ccccc3c2)c2ccc3cc4c(cc3c2)sc2ccccc24)cc1] 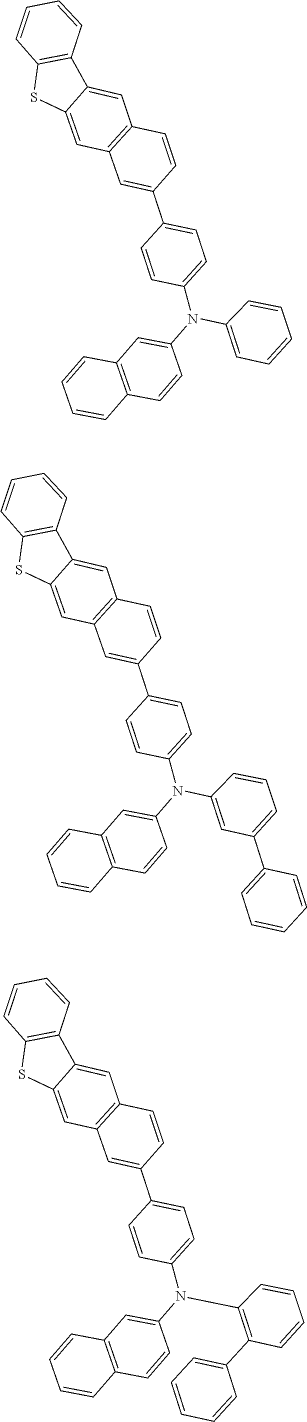 [SMILES: c1ccc(N(c2ccc(-c3ccc4cc5c(cc4c3)sc3ccccc35)cc2)c2ccc3ccccc3c2)cc1 ; c1ccc(-c2cccc(N(c3ccc(-c4ccc5cc6c(cc5c4)sc4ccccc46)cc3)c3ccc4ccccc4c3)c2)cc1 ; c1ccc(-c2ccccc2N(c2ccc(-c3ccc4cc5c(cc4c3)sc3ccccc35)cc2)c2ccc3ccccc3c2)cc1] 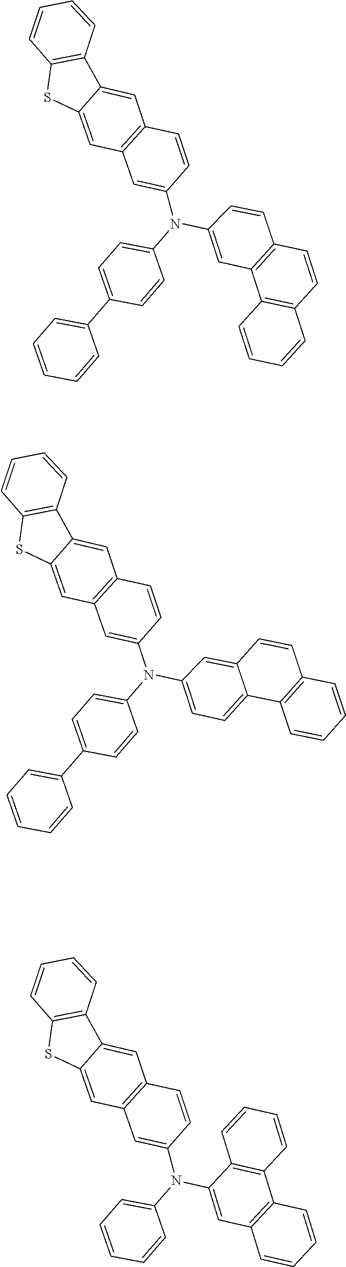 [SMILES: c1ccc(-c2ccc(N(c3ccc4cc5c(cc4c3)sc3ccccc35)c3ccc4ccc5ccccc5c4c3)cc2)cc1 ; c1ccc(-c2ccc(N(c3ccc4cc5c(cc4c3)sc3ccccc35)c3ccc4c(ccc5ccccc54)c3)cc2)cc1 ; c1ccc(N(c2ccc3cc4c(cc3c2)sc2ccccc24)c2cc3ccccc3c3ccccc23)cc1] 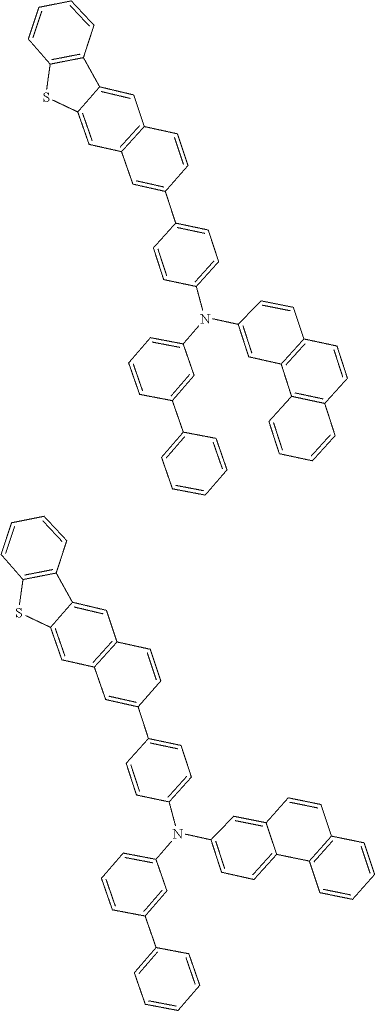 [SMILES: c1ccc(-c2cccc(N(c3ccc(-c4ccc5cc6c(cc5c4)sc4ccccc46)cc3)c3ccc4ccc5ccccc5c4c3)c2)cc1 ; c1ccc(-c2cccc(N(c3ccc(-c4ccc5cc6c(cc5c4)sc4ccccc46)cc3)c3ccc4c(ccc5ccccc54)c3)c2)cc1] 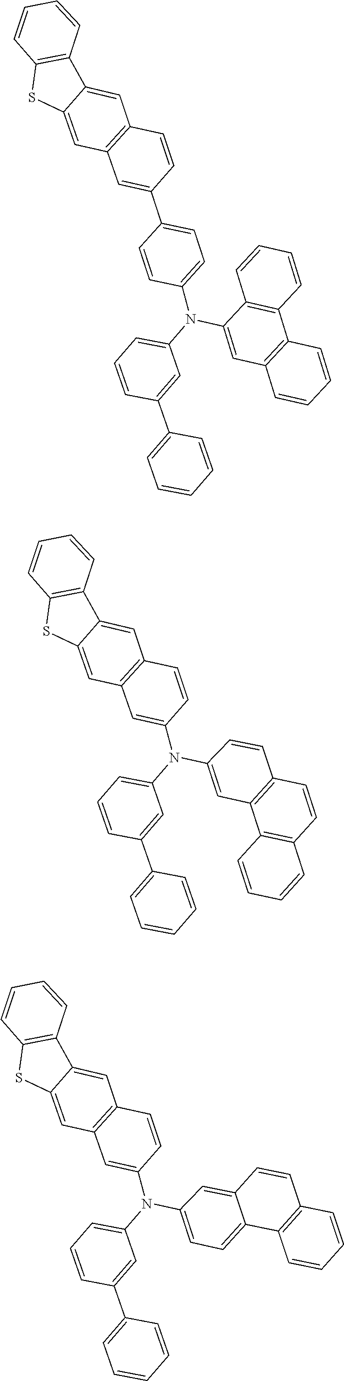 [SMILES: c1ccc(-c2cccc(N(c3ccc(-c4ccc5cc6c(cc5c4)sc4ccccc46)cc3)c3cc4ccccc4c4ccccc34)c2)cc1 ; c1ccc(-c2cccc(N(c3ccc4cc5c(cc4c3)sc3ccccc35)c3ccc4ccc5ccccc5c4c3)c2)cc1 ; c1ccc(-c2cccc(N(c3ccc4cc5c(cc4c3)sc3ccccc35)c3ccc4c(ccc5ccccc54)c3)c2)cc1] 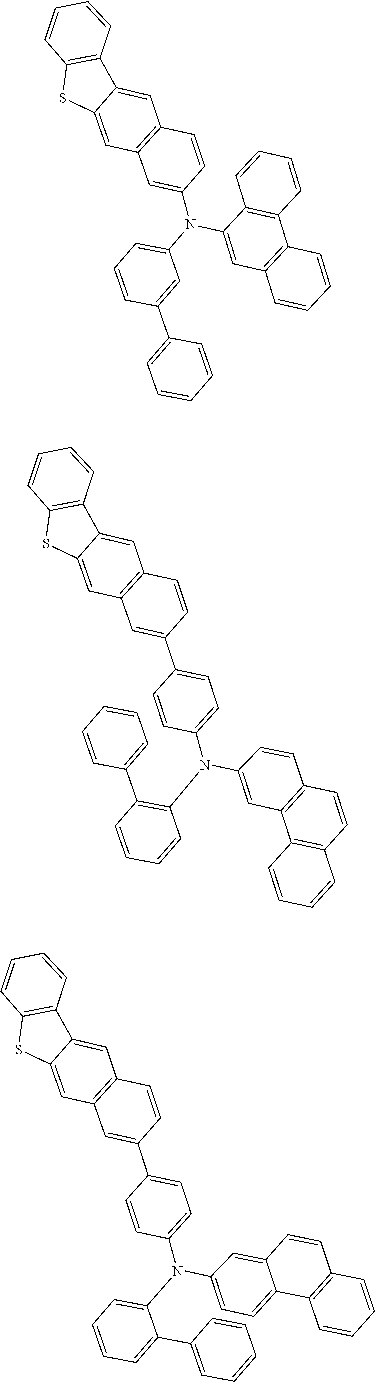 [SMILES: c1ccc(-c2cccc(N(c3ccc4cc5c(cc4c3)sc3ccccc35)c3cc4ccccc4c4ccccc34)c2)cc1 ; c1ccc(-c2ccccc2N(c2ccc(-c3ccc4cc5c(cc4c3)sc3ccccc35)cc2)c2ccc3ccc4ccccc4c3c2)cc1 ; c1ccc(-c2ccccc2N(c2ccc(-c3ccc4cc5c(cc4c3)sc3ccccc35)cc2)c2ccc3c(ccc4ccccc43)c2)cc1] 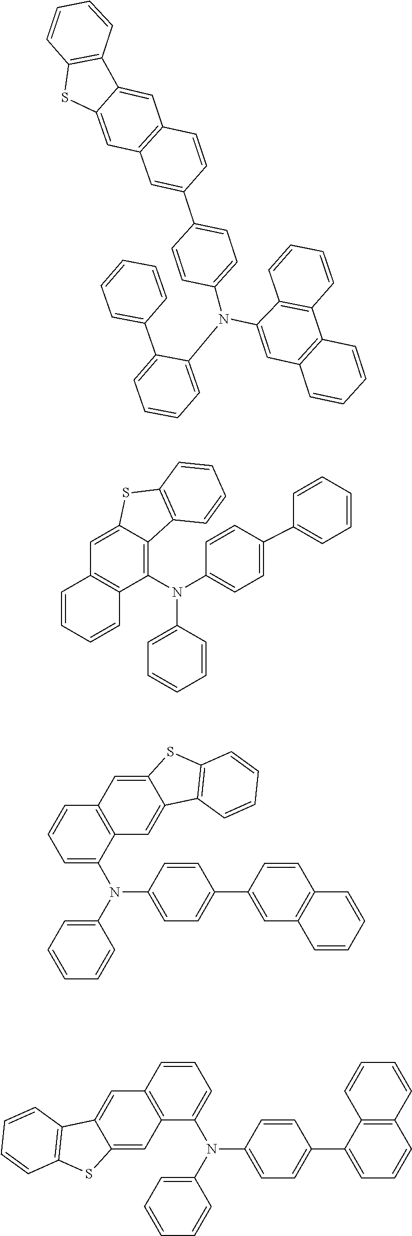 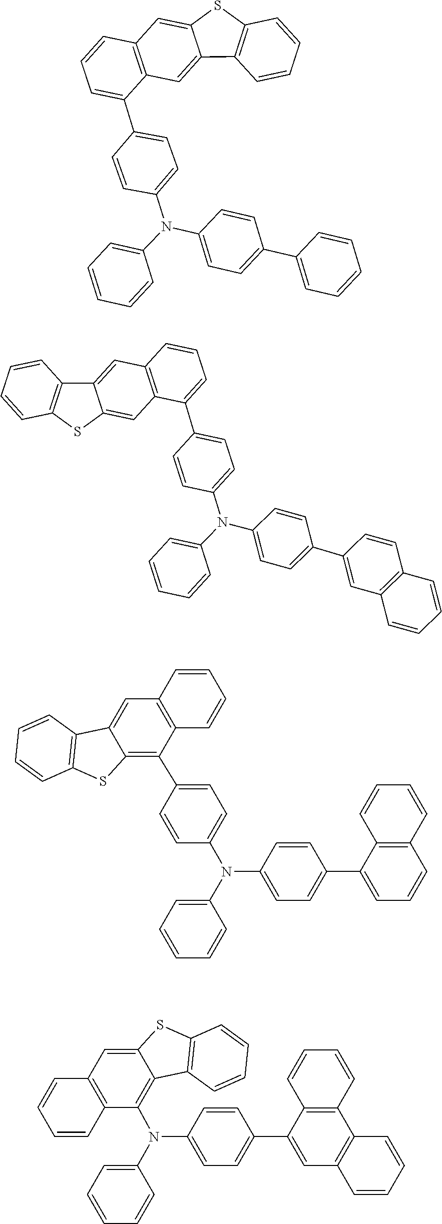 [SMILES: c1ccc(-c2ccc(N(c3ccccc3)c3ccc(-c4cccc5cc6sc7ccccc7c6cc45)cc3)cc2)cc1 ; c1ccc(N(c2ccc(-c3ccc4ccccc4c3)cc2)c2ccc(-c3cccc4cc5c(cc34)sc3ccccc35)cc2)cc1 ; c1ccc(N(c2ccc(-c3cccc4ccccc34)cc2)c2ccc(-c3c4ccccc4cc4c3sc3ccccc34)cc2)cc1 ; c1ccc(N(c2ccc(-c3cc4ccccc4c4ccccc34)cc2)c2c3ccccc3cc3sc4ccccc4c23)cc1] 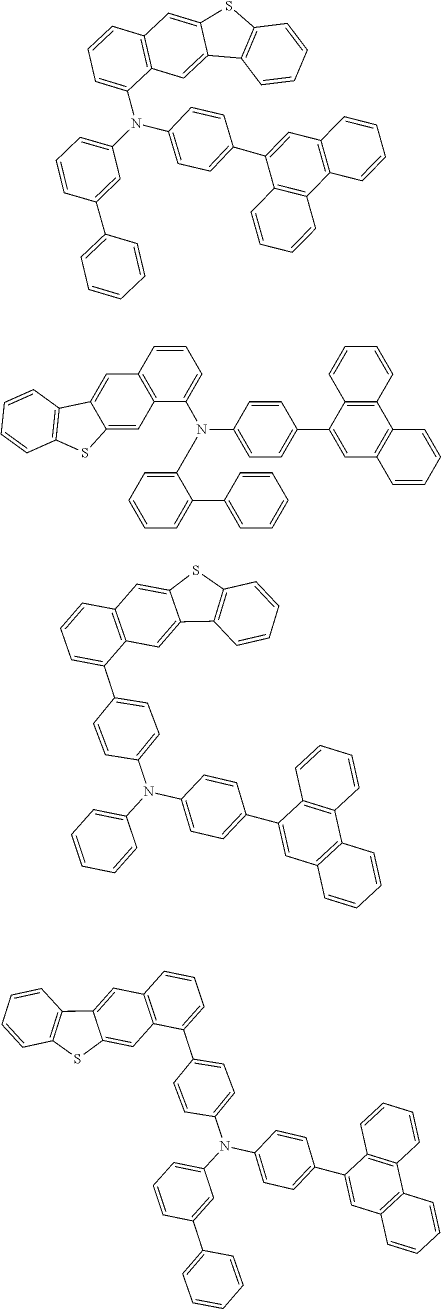 [SMILES: c1ccc(-c2cccc(N(c3ccc(-c4cc5ccccc5c5ccccc45)cc3)c3cccc4cc5sc6ccccc6c5cc34)c2)cc1 ; c1ccc(-c2ccccc2N(c2ccc(-c3cc4ccccc4c4ccccc34)cc2)c2cccc3cc4c(cc23)sc2ccccc24)cc1 ; c1ccc(N(c2ccc(-c3cccc4cc5sc6ccccc6c5cc34)cc2)c2ccc(-c3cc4ccccc4c4ccccc34)cc2)cc1 ; c1ccc(-c2cccc(N(c3ccc(-c4cccc5cc6c(cc45)sc4ccccc46)cc3)c3ccc(-c4cc5ccccc5c5ccccc45)cc3)c2)cc1] 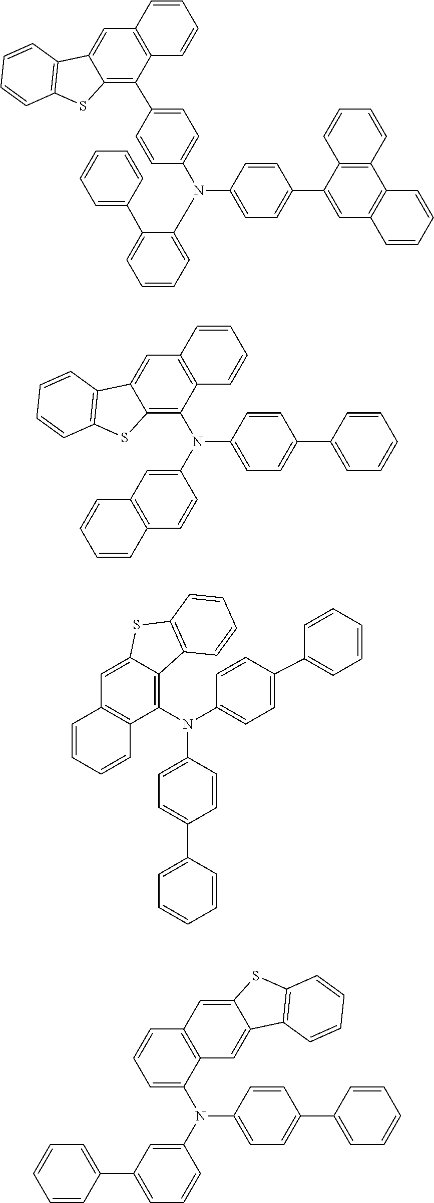 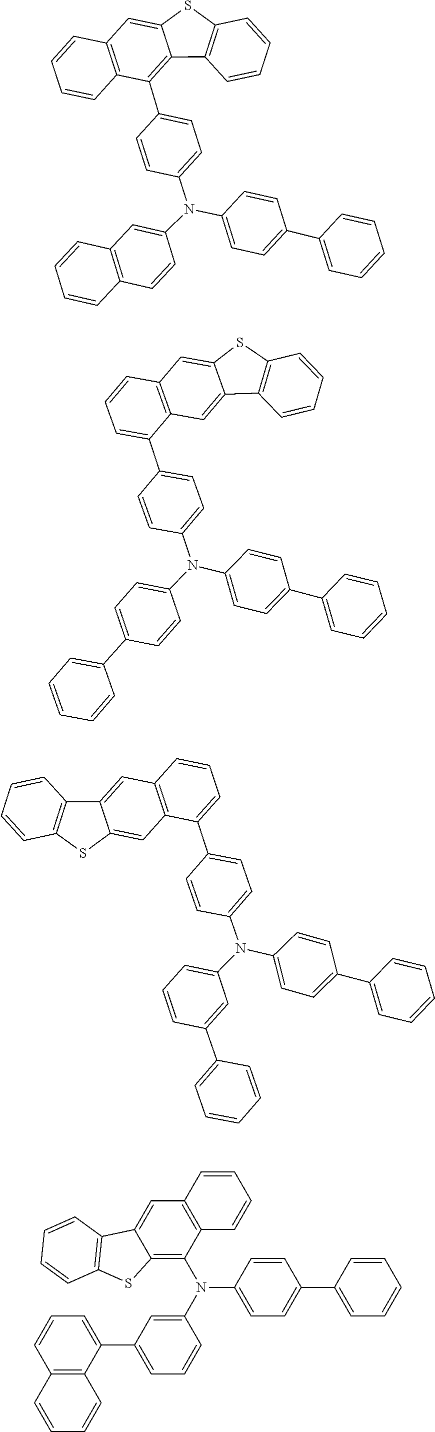 [SMILES: c1ccc(-c2ccc(N(c3ccc(-c4c5ccccc5cc5sc6ccccc6c45)cc3)c3ccc4ccccc4c3)cc2)cc1 ; c1ccc(-c2ccc(N(c3ccc(-c4ccccc4)cc3)c3ccc(-c4cccc5cc6sc7ccccc7c6cc45)cc3)cc2)cc1 ; c1ccc(-c2ccc(N(c3ccc(-c4cccc5cc6c(cc45)sc4ccccc46)cc3)c3cccc(-c4ccccc4)c3)cc2)cc1 ; c1ccc(-c2ccc(N(c3cccc(-c4cccc5ccccc45)c3)c3c4ccccc4cc4c3sc3ccccc34)cc2)cc1] 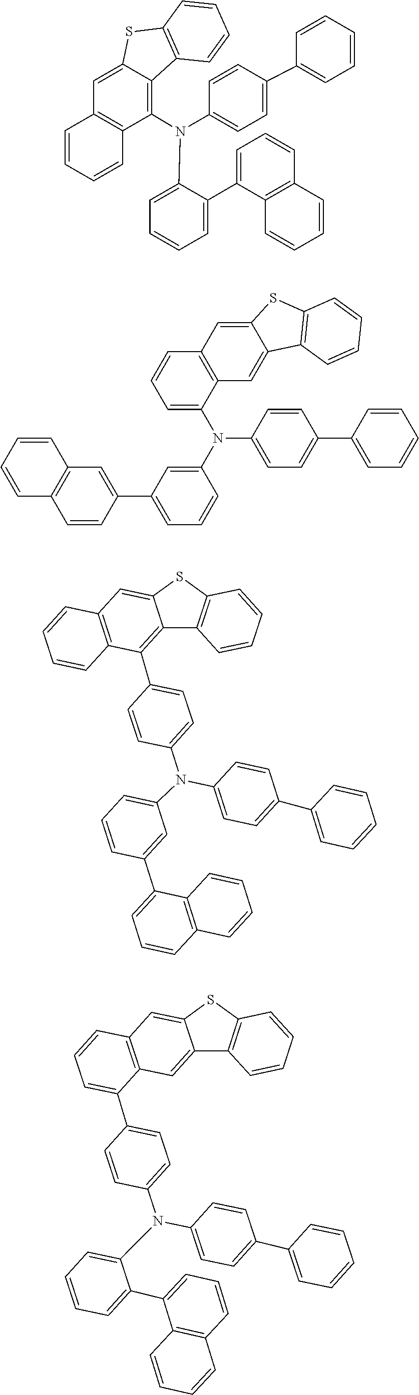 [SMILES: c1ccc(-c2ccc(N(c3ccccc3-c3cccc4ccccc34)c3c4ccccc4cc4sc5ccccc5c34)cc2)cc1 ; c1ccc(-c2ccc(N(c3cccc(-c4ccc5ccccc5c4)c3)c3cccc4cc5sc6ccccc6c5cc34)cc2)cc1 ; c1ccc(-c2ccc(N(c3ccc(-c4c5ccccc5cc5sc6ccccc6c45)cc3)c3cccc(-c4cccc5ccccc45)c3)cc2)cc1 ; c1ccc(-c2ccc(N(c3ccc(-c4cccc5cc6sc7ccccc7c6cc45)cc3)c3ccccc3-c3cccc4ccccc34)cc2)cc1] 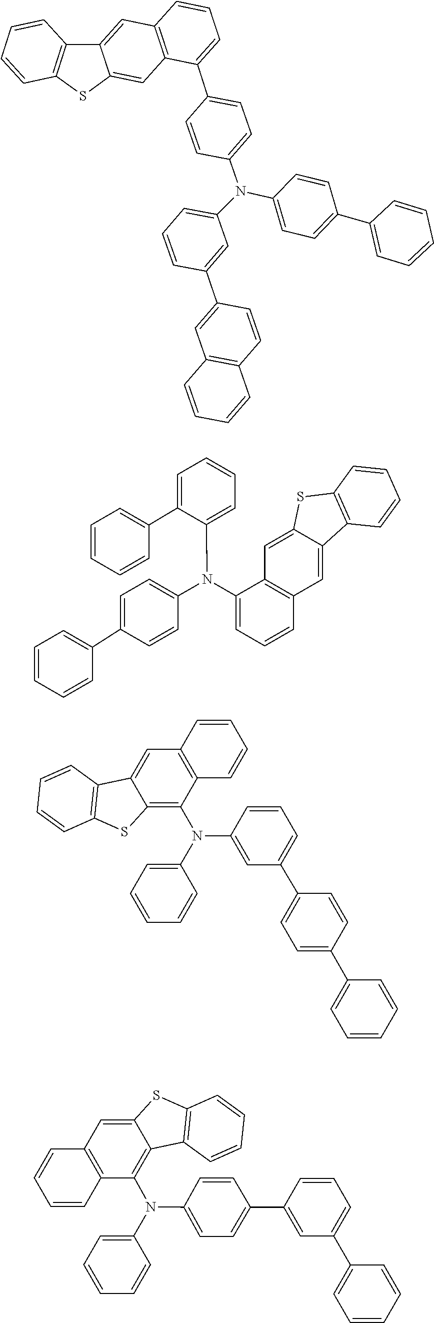 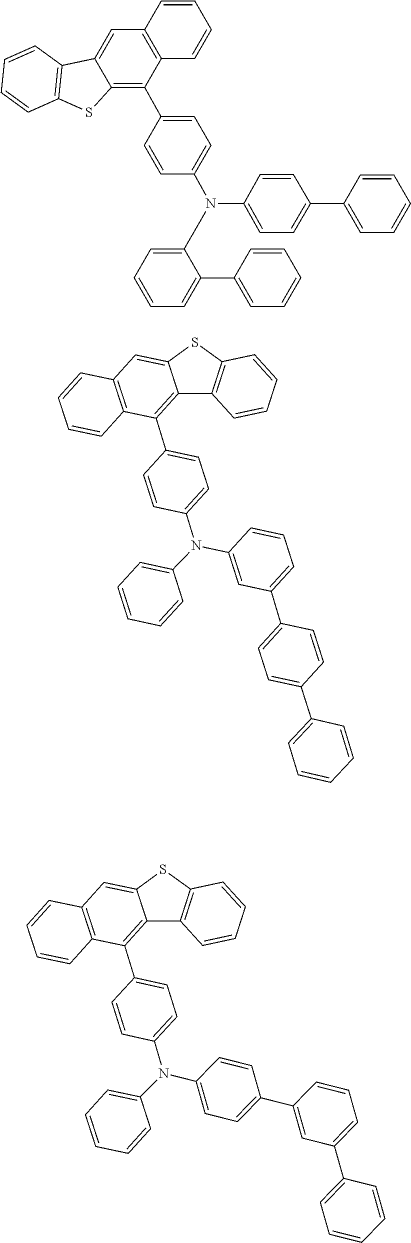 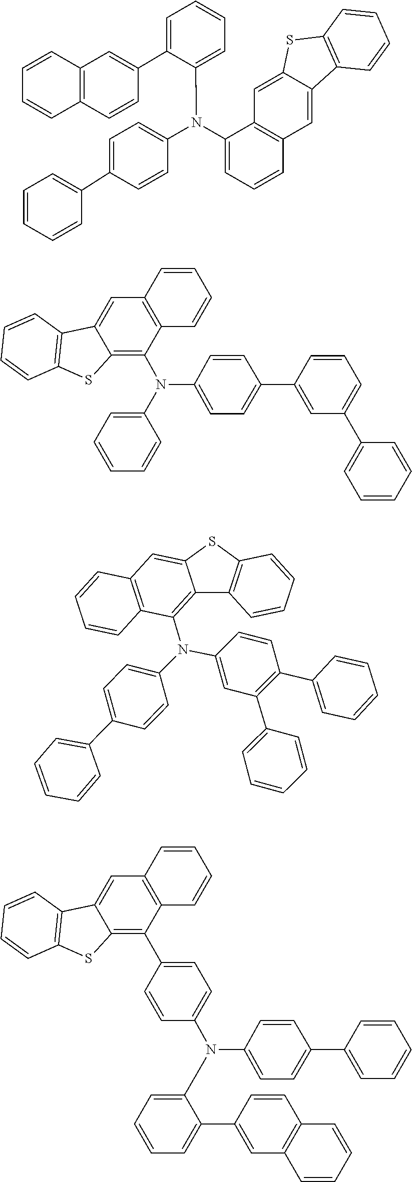 [SMILES: c1ccc(-c2ccc(N(c3ccccc3-c3ccc4ccccc4c3)c3cccc4cc5c(cc34)sc3ccccc35)cc2)cc1 ; c1ccc(-c2cccc(-c3ccc(N(c4ccccc4)c4c5ccccc5cc5c4sc4ccccc45)cc3)c2)cc1 ; c1ccc(-c2ccc(N(c3ccc(-c4ccccc4)c(-c4ccccc4)c3)c3c4ccccc4cc4sc5ccccc5c34)cc2)cc1 ; c1ccc(-c2ccc(N(c3ccc(-c4c5ccccc5cc5c4sc4ccccc45)cc3)c3ccccc3-c3ccc4ccccc4c3)cc2)cc1] 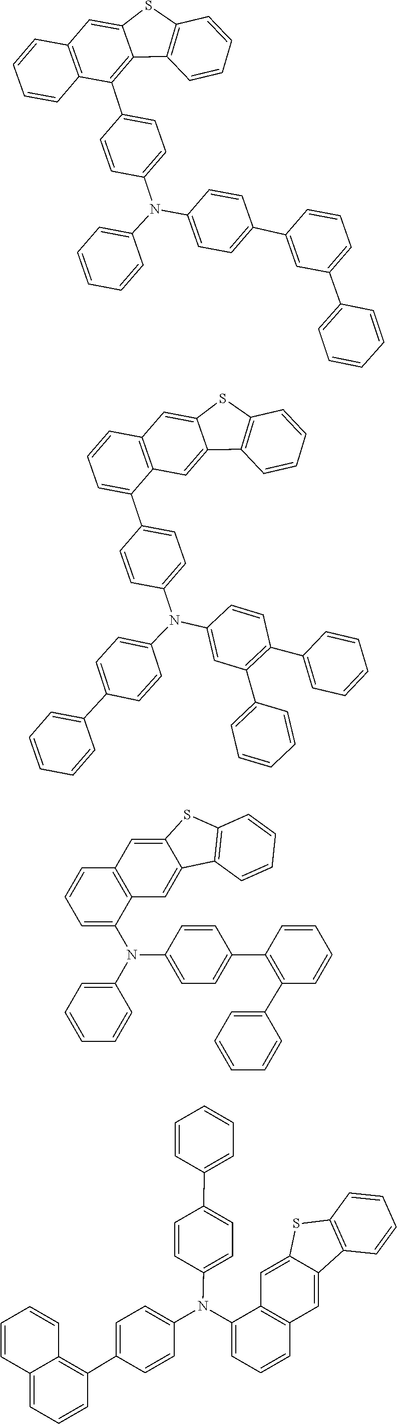 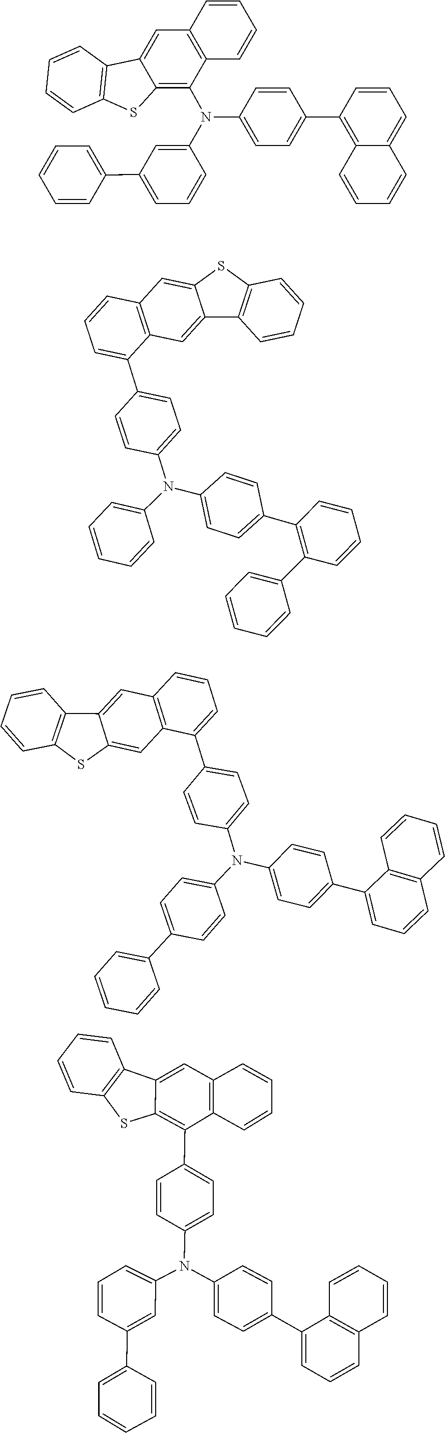 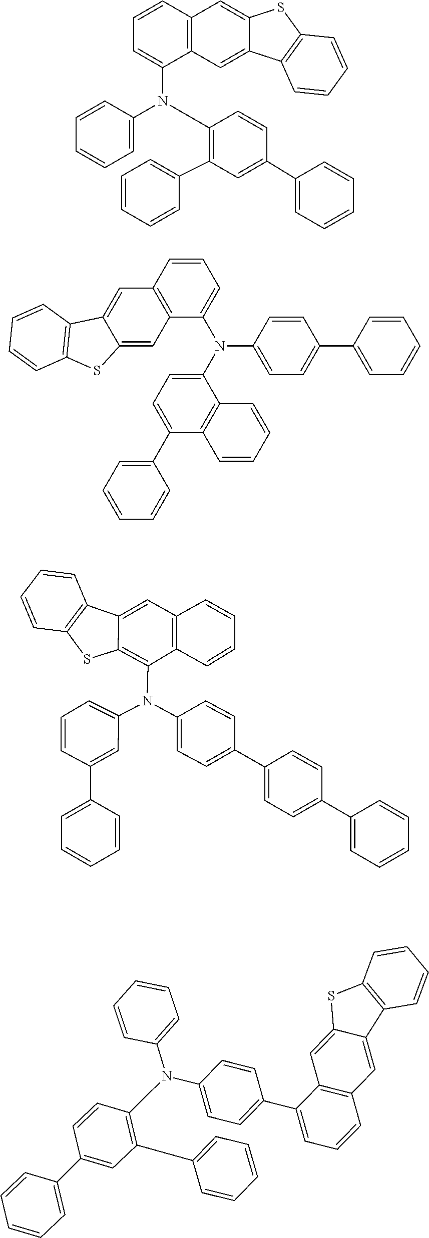 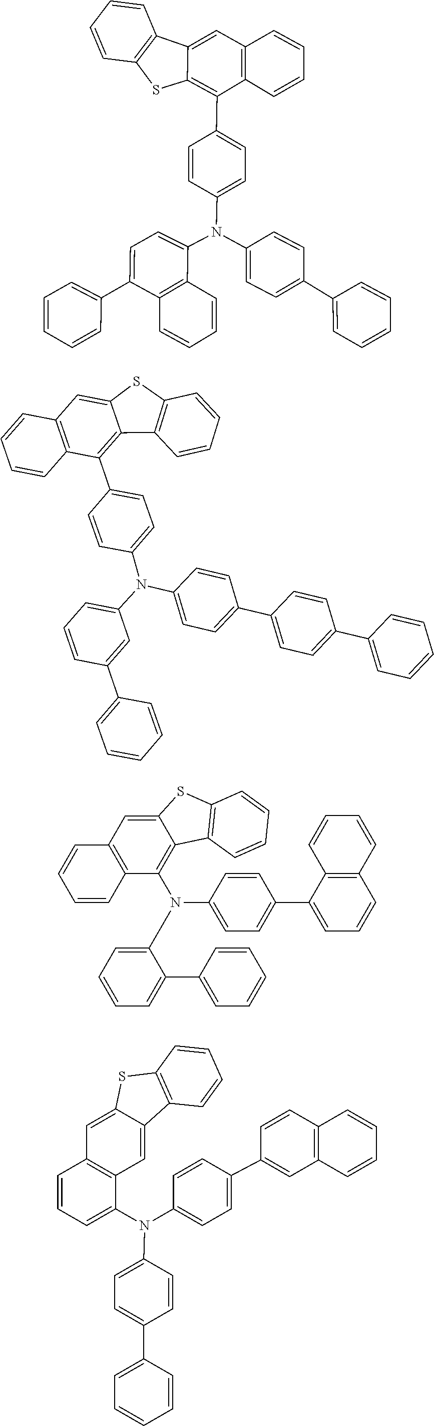 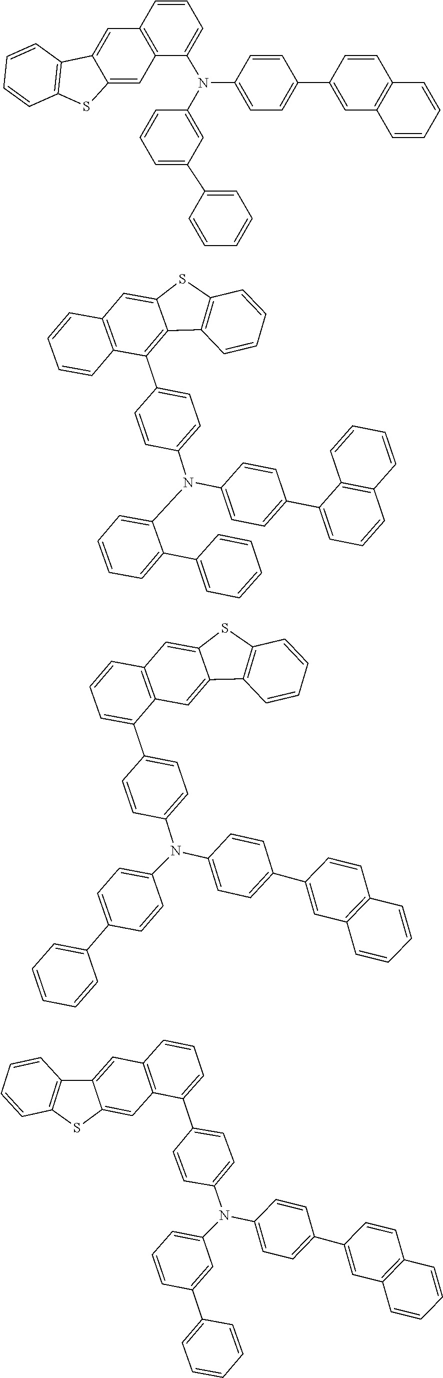 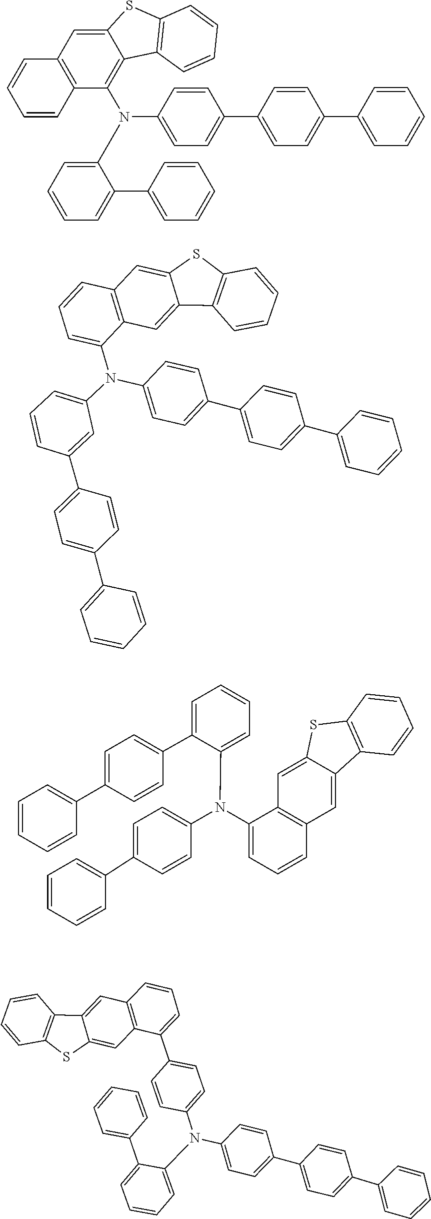 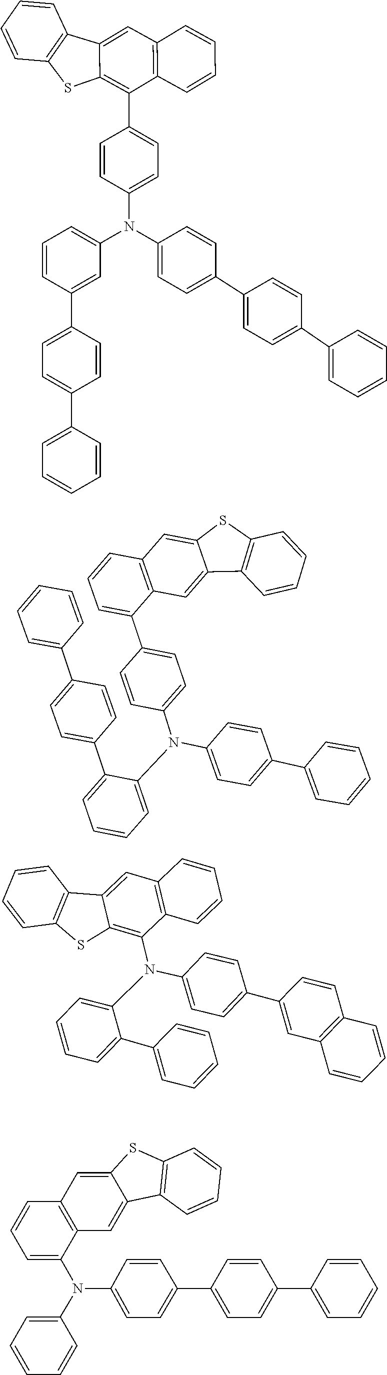 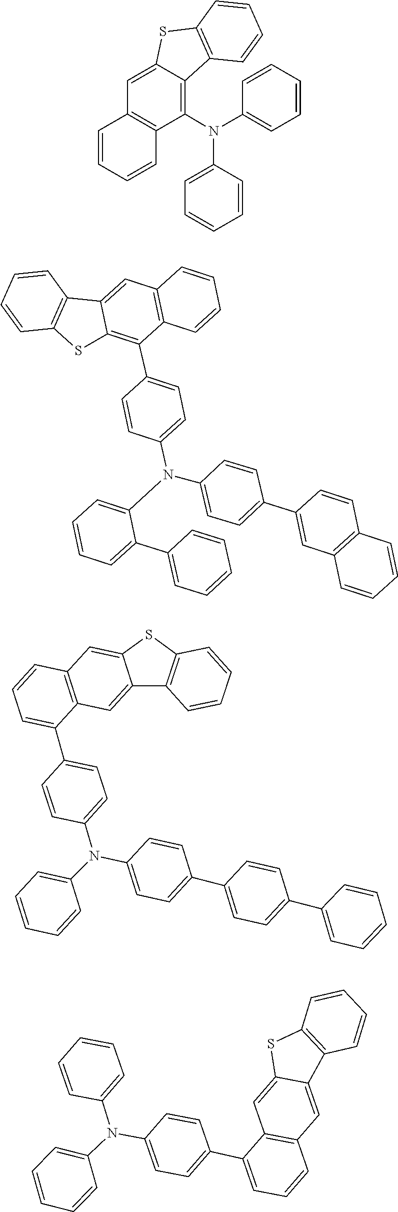 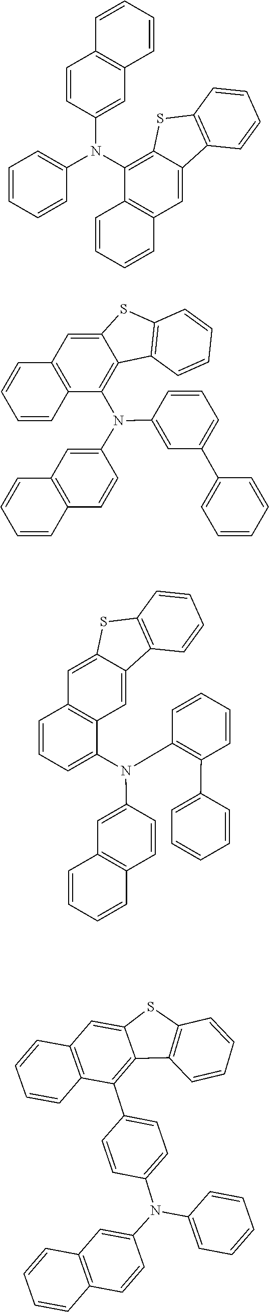 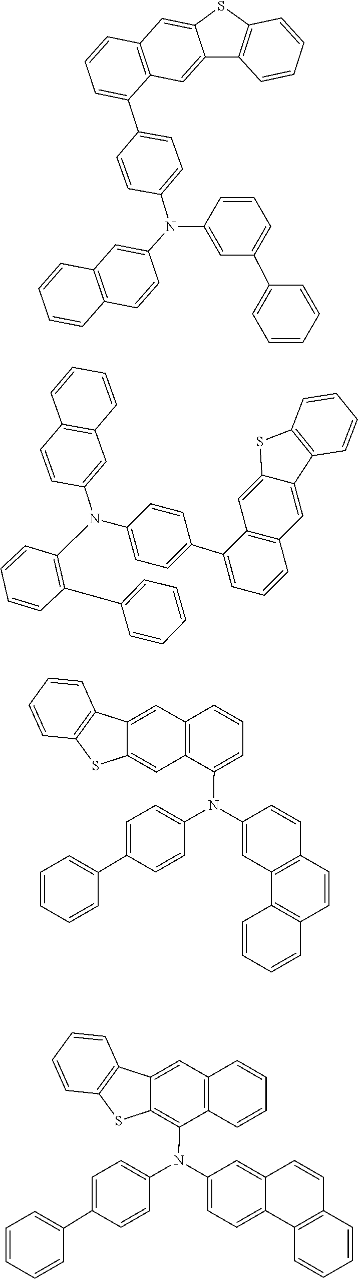 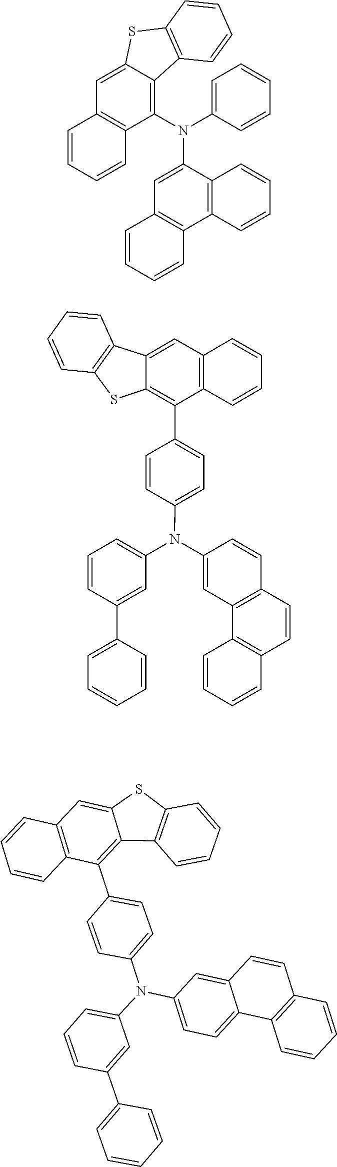 [SMILES: c1ccc(N(c2cc3ccccc3c3ccccc23)c2c3ccccc3cc3sc4ccccc4c23)cc1 ; c1ccc(-c2cccc(N(c3ccc(-c4c5ccccc5cc5c4sc4ccccc45)cc3)c3ccc4ccc5ccccc5c4c3)c2)cc1 ; c1ccc(-c2cccc(N(c3ccc(-c4c5ccccc5cc5sc6ccccc6c45)cc3)c3ccc4c(ccc5ccccc54)c3)c2)cc1] 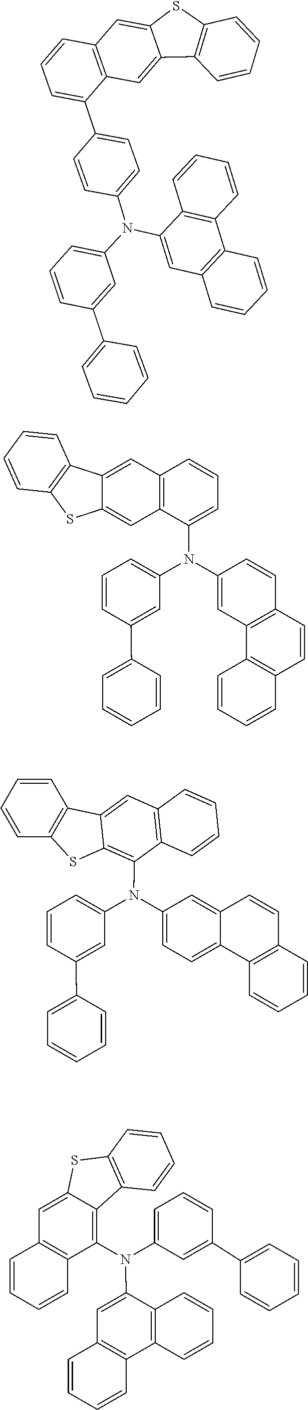 [SMILES: c1ccc(-c2cccc(N(c3ccc(-c4cccc5cc6sc7ccccc7c6cc45)cc3)c3cc4ccccc4c4ccccc34)c2)cc1 ; c1ccc(-c2cccc(N(c3ccc4ccc5ccccc5c4c3)c3cccc4cc5c(cc34)sc3ccccc35)c2)cc1 ; c1ccc(-c2cccc(N(c3ccc4c(ccc5ccccc54)c3)c3c4ccccc4cc4c3sc3ccccc34)c2)cc1 ; c1ccc(-c2cccc(N(c3cc4ccccc4c4ccccc34)c3c4ccccc4cc4sc5ccccc5c34)c2)cc1] 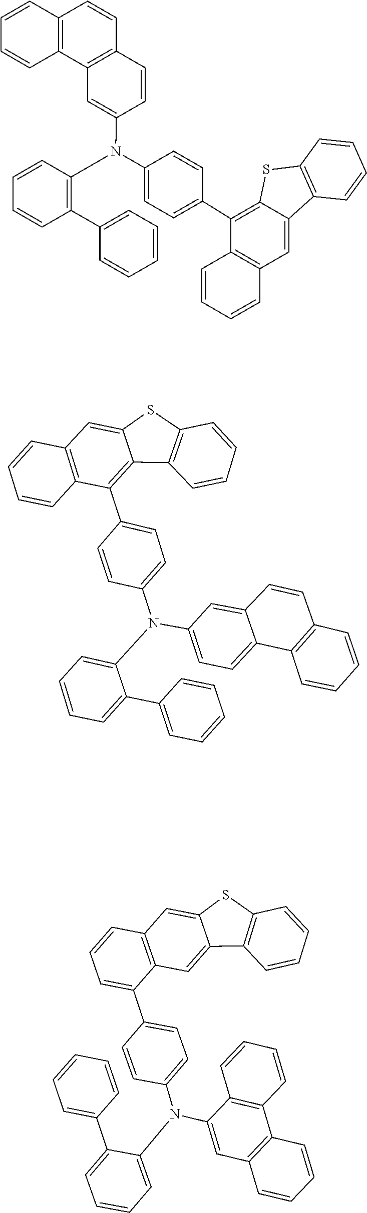 [SMILES: c1ccc(-c2ccccc2N(c2ccc(-c3c4ccccc4cc4c3sc3ccccc34)cc2)c2ccc3ccc4ccccc4c3c2)cc1 ; c1ccc(-c2ccccc2N(c2ccc(-c3c4ccccc4cc4sc5ccccc5c34)cc2)c2ccc3c(ccc4ccccc43)c2)cc1 ; c1ccc(-c2ccccc2N(c2ccc(-c3cccc4cc5sc6ccccc6c5cc34)cc2)c2cc3ccccc3c3ccccc23)cc1] 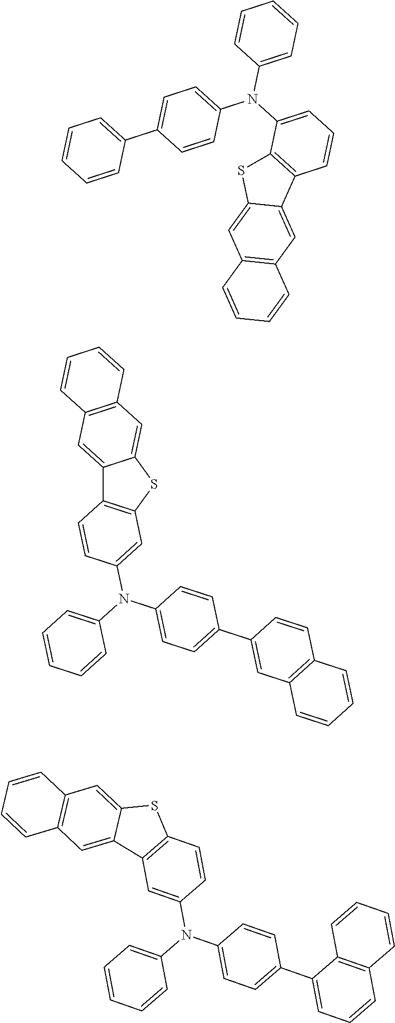 [SMILES: c1ccc(-c2ccc(N(c3ccccc3)c3cccc4c3sc3cc5ccccc5cc34)cc2)cc1 ; c1ccc(N(c2ccc(-c3ccc4ccccc4c3)cc2)c2ccc3c(c2)sc2cc4ccccc4cc23)cc1 ; c1ccc(N(c2ccc(-c3cccc4ccccc34)cc2)c2ccc3sc4cc5ccccc5cc4c3c2)cc1] 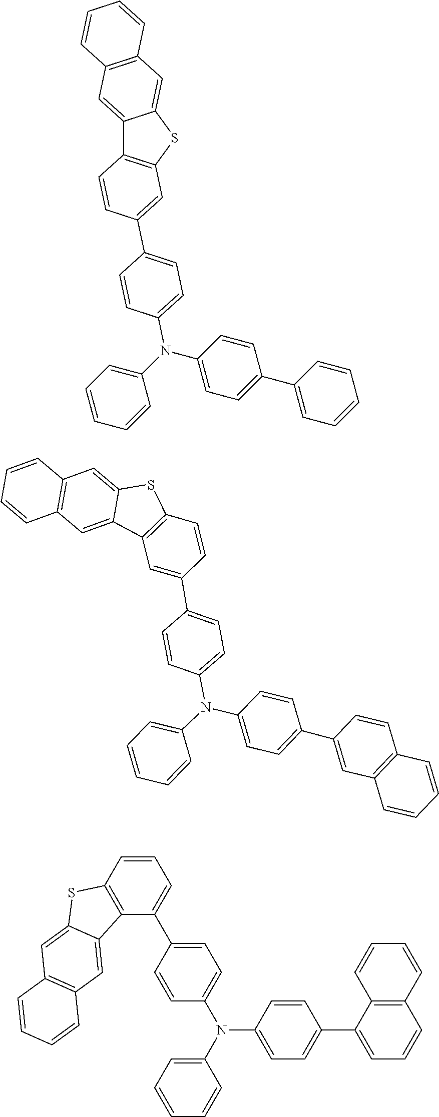 [SMILES: c1ccc(-c2ccc(N(c3ccccc3)c3ccc(-c4ccc5c(c4)sc4cc6ccccc6cc45)cc3)cc2)cc1 ; c1ccc(N(c2ccc(-c3ccc4ccccc4c3)cc2)c2ccc(-c3ccc4sc5cc6ccccc6cc5c4c3)cc2)cc1 ; c1ccc(N(c2ccc(-c3cccc4ccccc34)cc2)c2ccc(-c3cccc4sc5cc6ccccc6cc5c34)cc2)cc1] 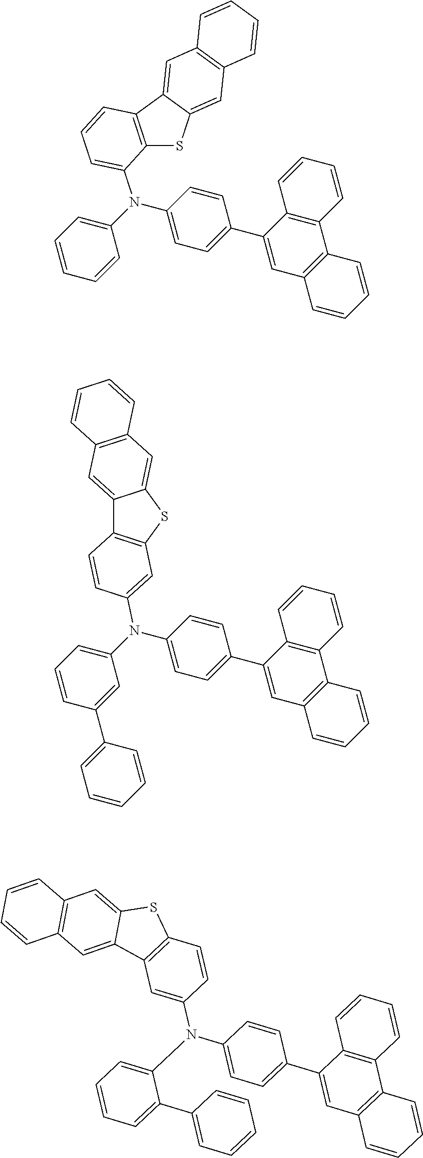 [SMILES: c1ccc(N(c2ccc(-c3cc4ccccc4c4ccccc34)cc2)c2cccc3c2sc2cc4ccccc4cc23)cc1 ; c1ccc(-c2cccc(N(c3ccc(-c4cc5ccccc5c5ccccc45)cc3)c3ccc4c(c3)sc3cc5ccccc5cc34)c2)cc1 ; c1ccc(-c2ccccc2N(c2ccc(-c3cc4ccccc4c4ccccc34)cc2)c2ccc3sc4cc5ccccc5cc4c3c2)cc1] 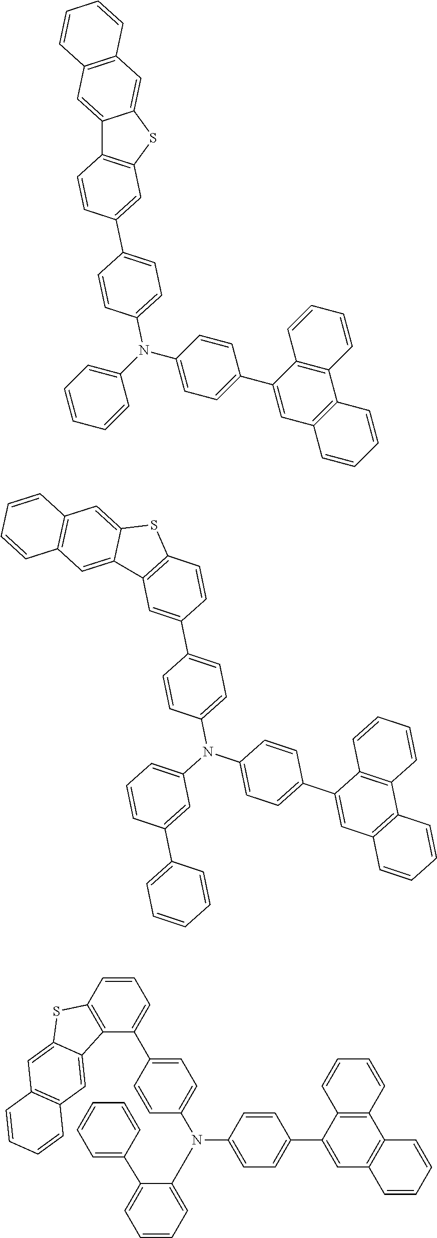 [SMILES: c1ccc(N(c2ccc(-c3ccc4c(c3)sc3cc5ccccc5cc34)cc2)c2ccc(-c3cc4ccccc4c4ccccc34)cc2)cc1 ; c1ccc(-c2cccc(N(c3ccc(-c4ccc5sc6cc7ccccc7cc6c5c4)cc3)c3ccc(-c4cc5ccccc5c5ccccc45)cc3)c2)cc1 ; c1ccc(-c2ccccc2N(c2ccc(-c3cc4ccccc4c4ccccc34)cc2)c2ccc(-c3cccc4sc5cc6ccccc6cc5c34)cc2)cc1] 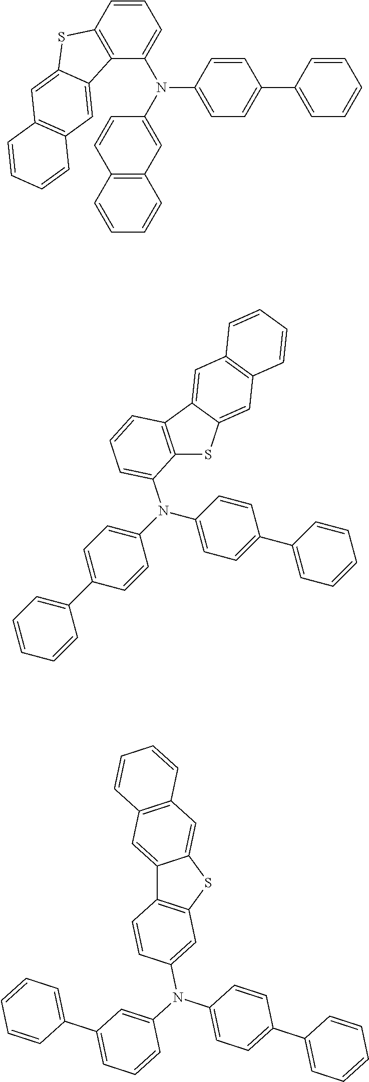 [SMILES: c1ccc(-c2ccc(N(c3ccc4ccccc4c3)c3cccc4sc5cc6ccccc6cc5c34)cc2)cc1 ; c1ccc(-c2ccc(N(c3ccc(-c4ccccc4)cc3)c3cccc4c3sc3cc5ccccc5cc34)cc2)cc1 ; c1ccc(-c2ccc(N(c3cccc(-c4ccccc4)c3)c3ccc4c(c3)sc3cc5ccccc5cc34)cc2)cc1] 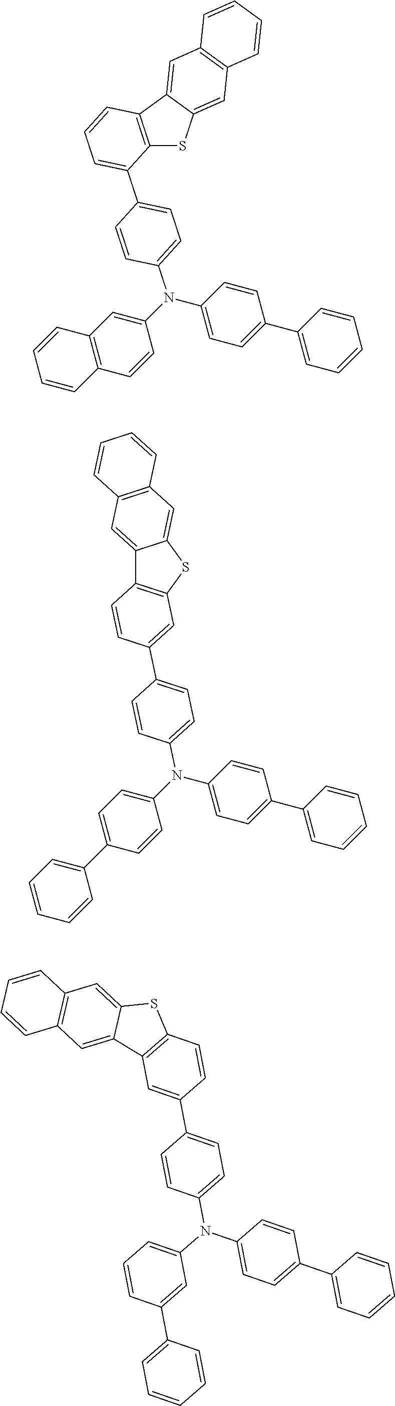 [SMILES: c1ccc(-c2ccc(N(c3ccc(-c4cccc5c4sc4cc6ccccc6cc45)cc3)c3ccc4ccccc4c3)cc2)cc1 ; c1ccc(-c2ccc(N(c3ccc(-c4ccccc4)cc3)c3ccc(-c4ccc5c(c4)sc4cc6ccccc6cc45)cc3)cc2)cc1 ; c1ccc(-c2ccc(N(c3ccc(-c4ccc5sc6cc7ccccc7cc6c5c4)cc3)c3cccc(-c4ccccc4)c3)cc2)cc1] 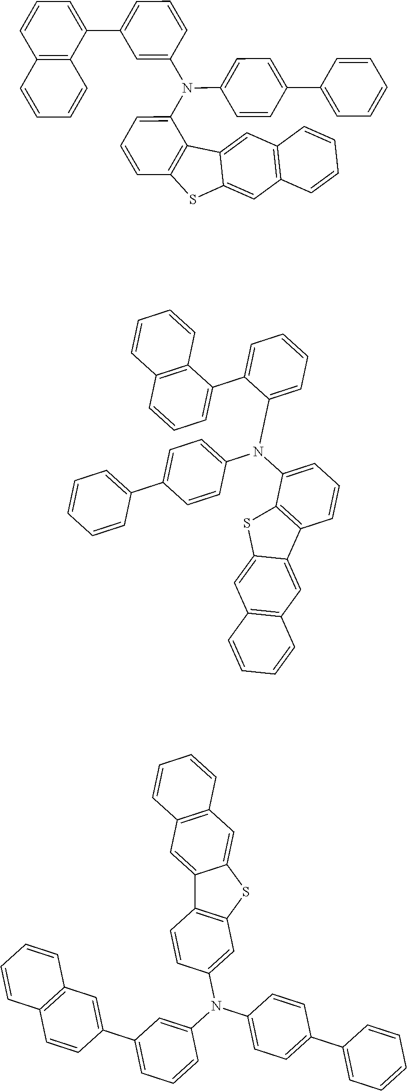 [SMILES: c1ccc(-c2ccc(N(c3cccc(-c4cccc5ccccc45)c3)c3cccc4sc5cc6ccccc6cc5c34)cc2)cc1 ; c1ccc(-c2ccc(N(c3ccccc3-c3cccc4ccccc34)c3cccc4c3sc3cc5ccccc5cc34)cc2)cc1 ; c1ccc(-c2ccc(N(c3cccc(-c4ccc5ccccc5c4)c3)c3ccc4c(c3)sc3cc5ccccc5cc34)cc2)cc1] 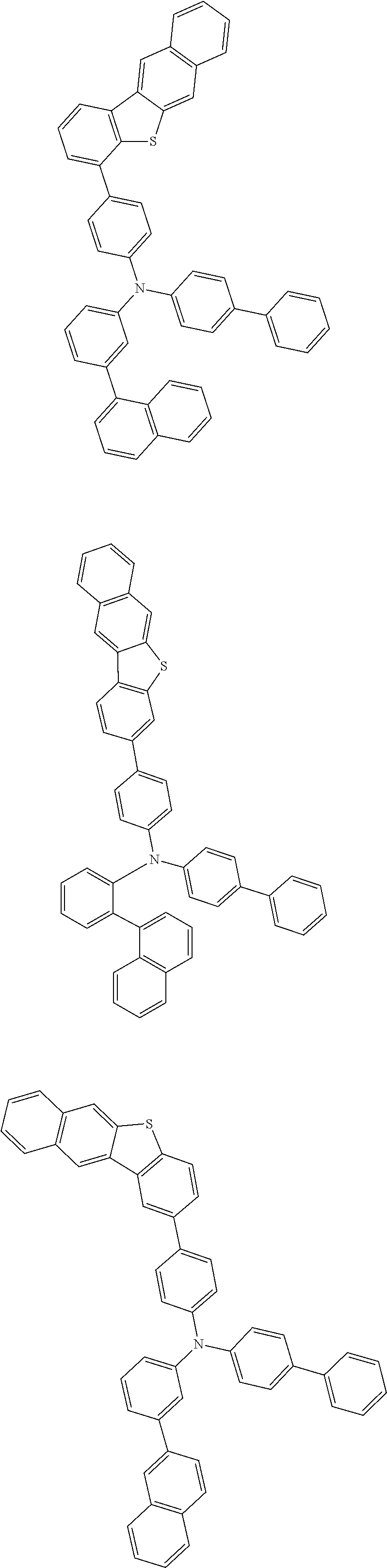 [SMILES: c1ccc(-c2ccc(N(c3ccc(-c4cccc5c4sc4cc6ccccc6cc45)cc3)c3cccc(-c4cccc5ccccc45)c3)cc2)cc1 ; c1ccc(-c2ccc(N(c3ccc(-c4ccc5c(c4)sc4cc6ccccc6cc45)cc3)c3ccccc3-c3cccc4ccccc34)cc2)cc1 ; c1ccc(-c2ccc(N(c3ccc(-c4ccc5sc6cc7ccccc7cc6c5c4)cc3)c3cccc(-c4ccc5ccccc5c4)c3)cc2)cc1] 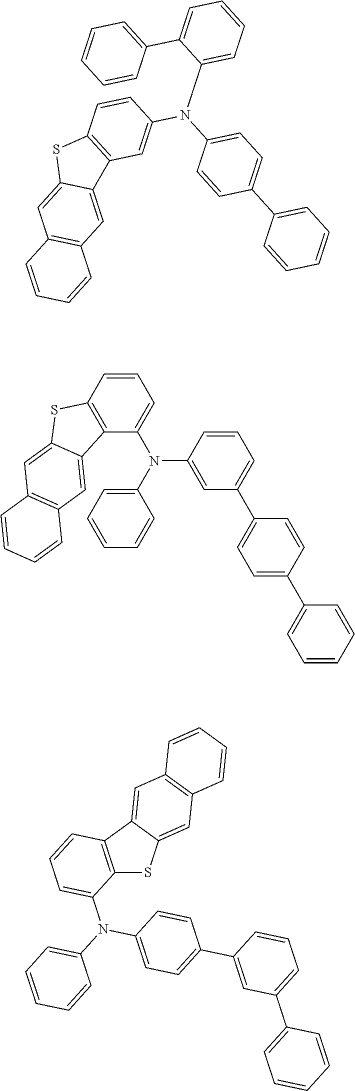 [SMILES: c1ccc(-c2ccc(N(c3ccc4sc5cc6ccccc6cc5c4c3)c3ccccc3-c3ccccc3)cc2)cc1 ; c1ccc(-c2ccc(-c3cccc(N(c4ccccc4)c4cccc5sc6cc7ccccc7cc6c45)c3)cc2)cc1 ; c1ccc(-c2cccc(-c3ccc(N(c4ccccc4)c4cccc5c4sc4cc6ccccc6cc45)cc3)c2)cc1] 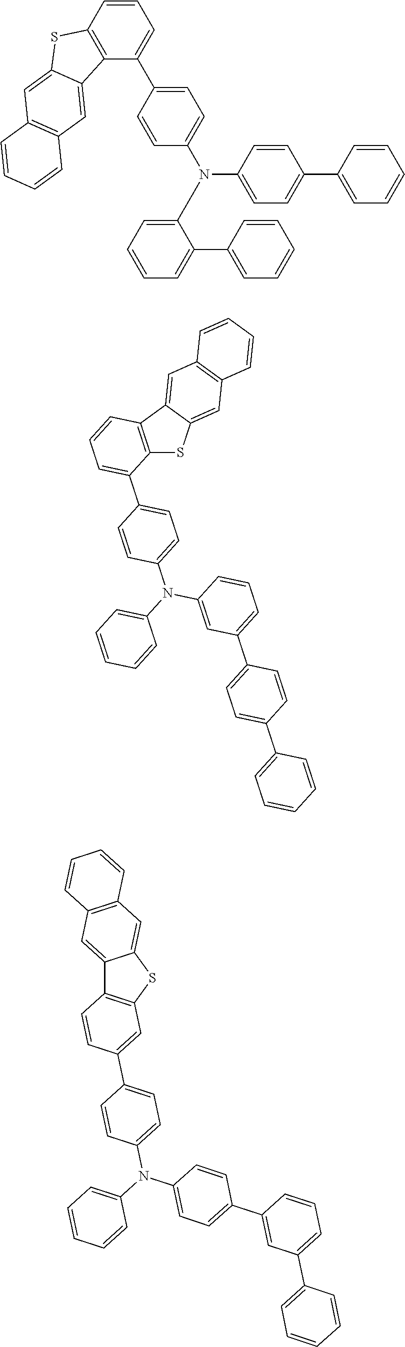 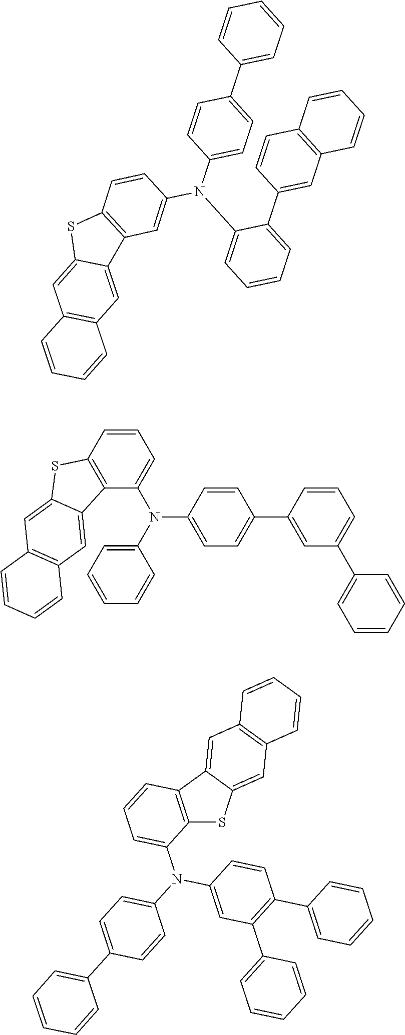 [SMILES: c1ccc(-c2ccc(N(c3ccc4sc5cc6ccccc6cc5c4c3)c3ccccc3-c3ccc4ccccc4c3)cc2)cc1 ; c1ccc(-c2cccc(-c3ccc(N(c4ccccc4)c4cccc5sc6cc7ccccc7cc6c45)cc3)c2)cc1 ; c1ccc(-c2ccc(N(c3ccc(-c4ccccc4)c(-c4ccccc4)c3)c3cccc4c3sc3cc5ccccc5cc34)cc2)cc1] 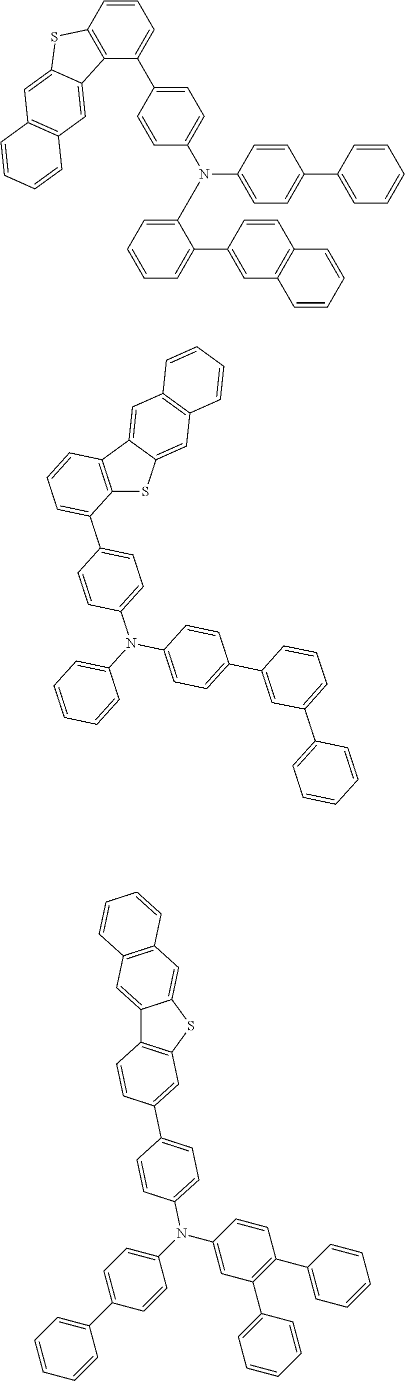 [SMILES: c1ccc(-c2ccc(N(c3ccc(-c4cccc5sc6cc7ccccc7cc6c45)cc3)c3ccccc3-c3ccc4ccccc4c3)cc2)cc1 ; c1ccc(-c2cccc(-c3ccc(N(c4ccccc4)c4ccc(-c5cccc6c5sc5cc7ccccc7cc56)cc4)cc3)c2)cc1 ; c1ccc(-c2ccc(N(c3ccc(-c4ccc5c(c4)sc4cc6ccccc6cc45)cc3)c3ccc(-c4ccccc4)c(-c4ccccc4)c3)cc2)cc1] 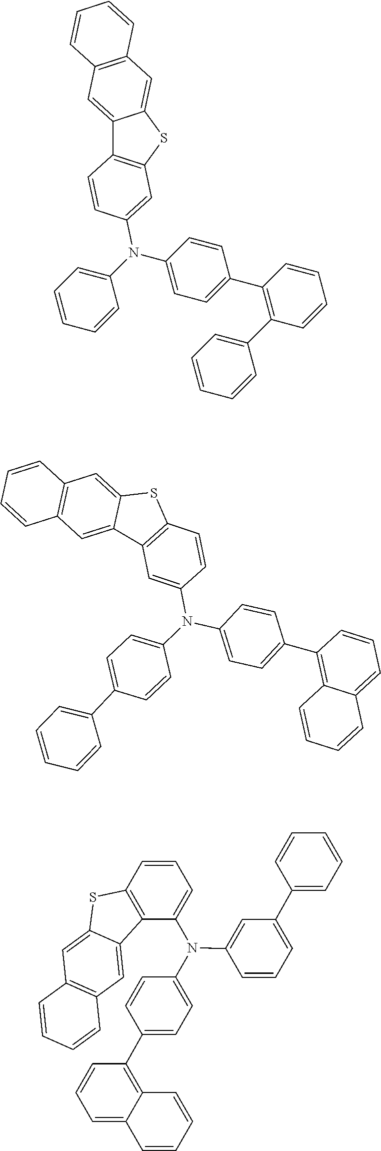 [SMILES: c1ccc(-c2ccccc2-c2ccc(N(c3ccccc3)c3ccc4c(c3)sc3cc5ccccc5cc34)cc2)cc1 ; c1ccc(-c2ccc(N(c3ccc(-c4cccc5ccccc45)cc3)c3ccc4sc5cc6ccccc6cc5c4c3)cc2)cc1 ; c1ccc(-c2cccc(N(c3ccc(-c4cccc5ccccc45)cc3)c3cccc4sc5cc6ccccc6cc5c34)c2)cc1] 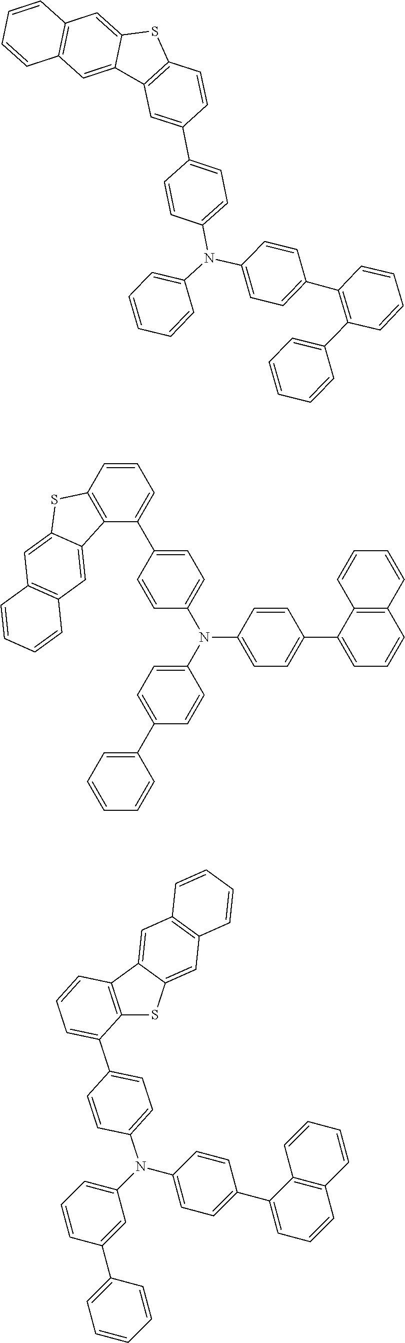 [SMILES: c1ccc(-c2ccccc2-c2ccc(N(c3ccccc3)c3ccc(-c4ccc5sc6cc7ccccc7cc6c5c4)cc3)cc2)cc1 ; c1ccc(-c2ccc(N(c3ccc(-c4cccc5ccccc45)cc3)c3ccc(-c4cccc5sc6cc7ccccc7cc6c45)cc3)cc2)cc1 ; c1ccc(-c2cccc(N(c3ccc(-c4cccc5ccccc45)cc3)c3ccc(-c4cccc5c4sc4cc6ccccc6cc45)cc3)c2)cc1] 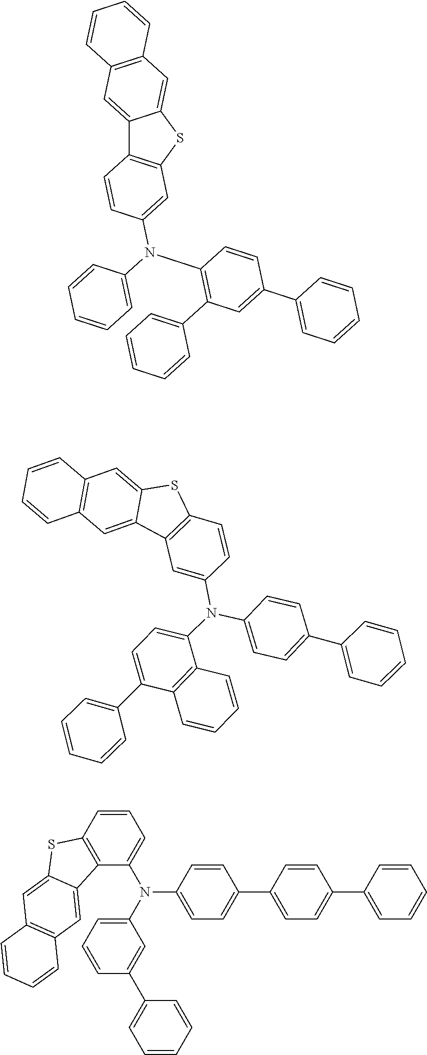 [SMILES: c1ccc(-c2ccc(N(c3ccccc3)c3ccc4c(c3)sc3cc5ccccc5cc34)c(-c3ccccc3)c2)cc1 ; c1ccc(-c2ccc(N(c3ccc4sc5cc6ccccc6cc5c4c3)c3ccc(-c4ccccc4)c4ccccc34)cc2)cc1 ; c1ccc(-c2ccc(-c3ccc(N(c4cccc(-c5ccccc5)c4)c4cccc5sc6cc7ccccc7cc6c45)cc3)cc2)cc1] 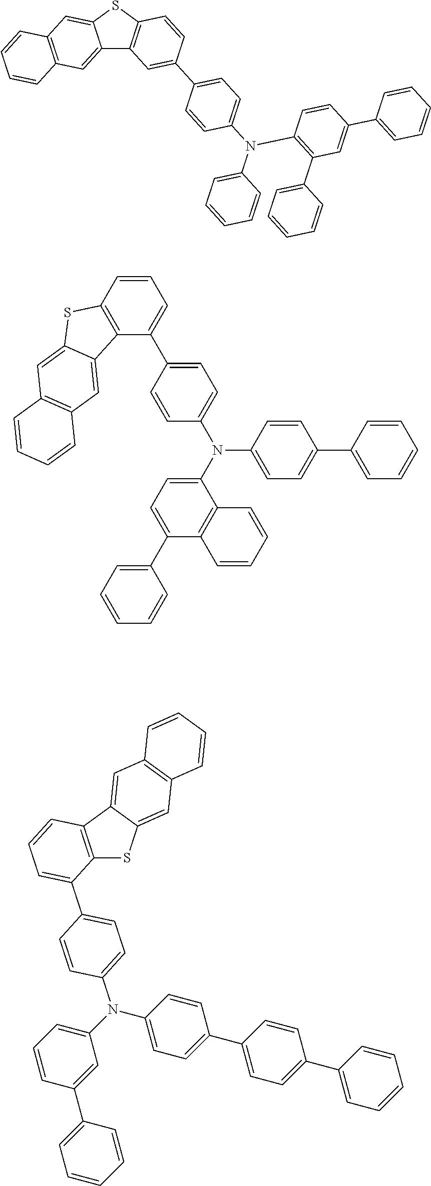 [SMILES: c1ccc(-c2ccc(N(c3ccccc3)c3ccc(-c4ccc5sc6cc7ccccc7cc6c5c4)cc3)c(-c3ccccc3)c2)cc1 ; c1ccc(-c2ccc(N(c3ccc(-c4cccc5sc6cc7ccccc7cc6c45)cc3)c3ccc(-c4ccccc4)c4ccccc34)cc2)cc1 ; c1ccc(-c2ccc(-c3ccc(N(c4ccc(-c5cccc6c5sc5cc7ccccc7cc56)cc4)c4cccc(-c5ccccc5)c4)cc3)cc2)cc1] 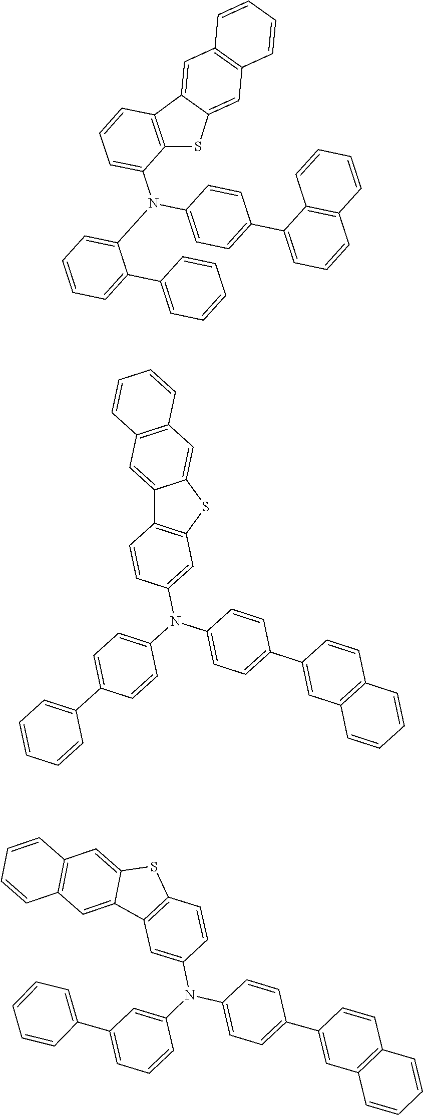 [SMILES: c1ccc(-c2ccccc2N(c2ccc(-c3cccc4ccccc34)cc2)c2cccc3c2sc2cc4ccccc4cc23)cc1 ; c1ccc(-c2ccc(N(c3ccc(-c4ccc5ccccc5c4)cc3)c3ccc4c(c3)sc3cc5ccccc5cc34)cc2)cc1 ; c1ccc(-c2cccc(N(c3ccc(-c4ccc5ccccc5c4)cc3)c3ccc4sc5cc6ccccc6cc5c4c3)c2)cc1] 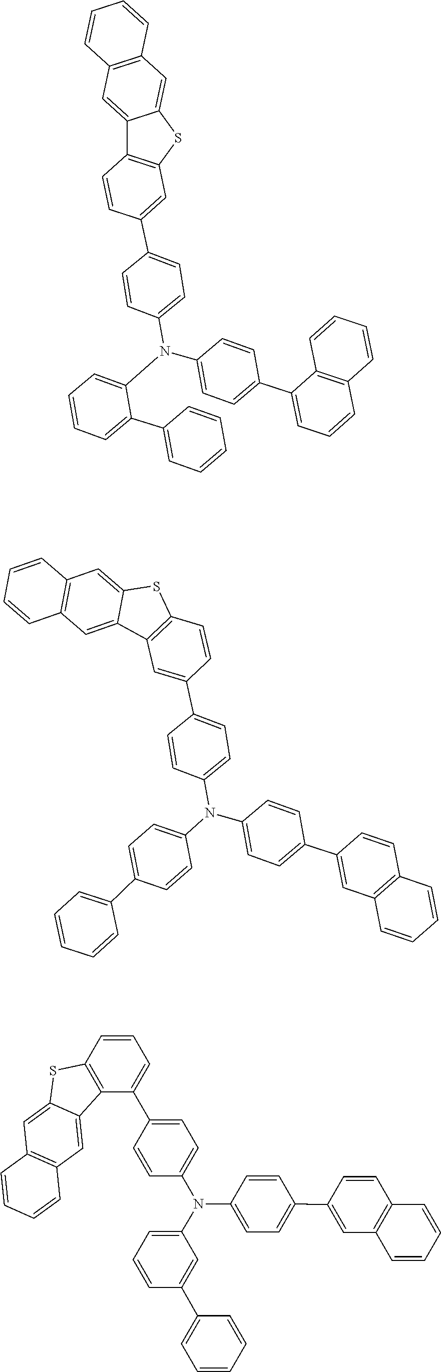 [SMILES: c1ccc(-c2ccccc2N(c2ccc(-c3ccc4c(c3)sc3cc5ccccc5cc34)cc2)c2ccc(-c3cccc4ccccc34)cc2)cc1 ; c1ccc(-c2ccc(N(c3ccc(-c4ccc5ccccc5c4)cc3)c3ccc(-c4ccc5sc6cc7ccccc7cc6c5c4)cc3)cc2)cc1 ; c1ccc(-c2cccc(N(c3ccc(-c4ccc5ccccc5c4)cc3)c3ccc(-c4cccc5sc6cc7ccccc7cc6c45)cc3)c2)cc1] 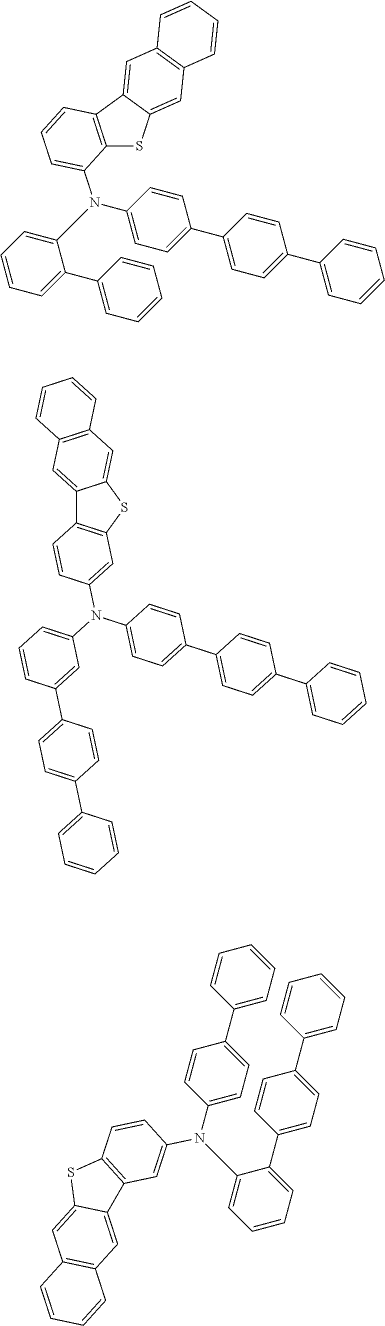 [SMILES: c1ccc(-c2ccc(-c3ccc(N(c4ccccc4-c4ccccc4)c4cccc5c4sc4cc6ccccc6cc45)cc3)cc2)cc1 ; c1ccc(-c2ccc(-c3ccc(N(c4cccc(-c5ccc(-c6ccccc6)cc5)c4)c4ccc5c(c4)sc4cc6ccccc6cc45)cc3)cc2)cc1 ; c1ccc(-c2ccc(-c3ccccc3N(c3ccc(-c4ccccc4)cc3)c3ccc4sc5cc6ccccc6cc5c4c3)cc2)cc1] 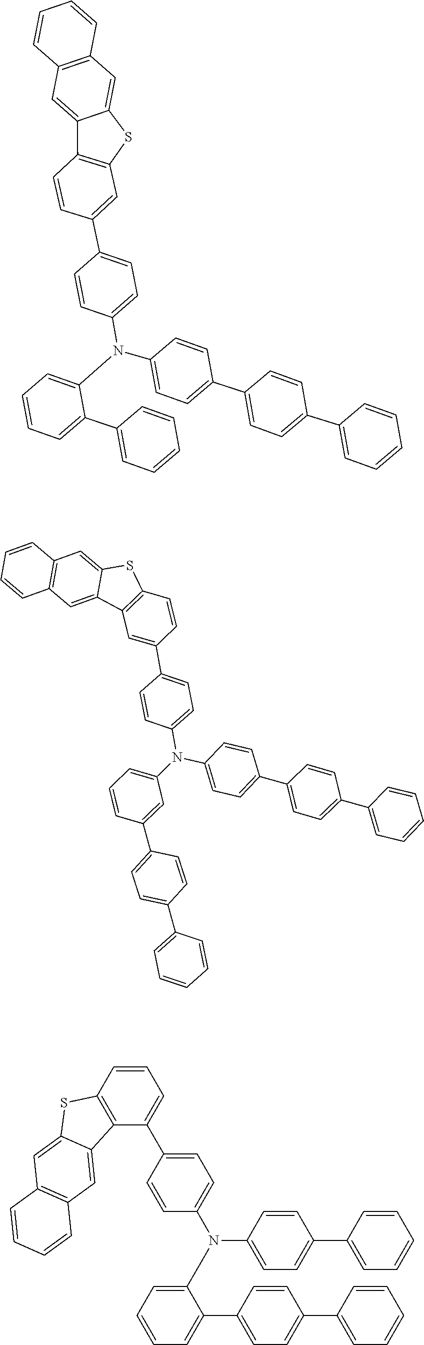 [SMILES: c1ccc(-c2ccc(-c3ccc(N(c4ccc(-c5ccc6c(c5)sc5cc7ccccc7cc56)cc4)c4ccccc4-c4ccccc4)cc3)cc2)cc1 ; c1ccc(-c2ccc(-c3ccc(N(c4ccc(-c5ccc6sc7cc8ccccc8cc7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5sc6cc7ccccc7cc6c45)cc3)cc2)cc1] 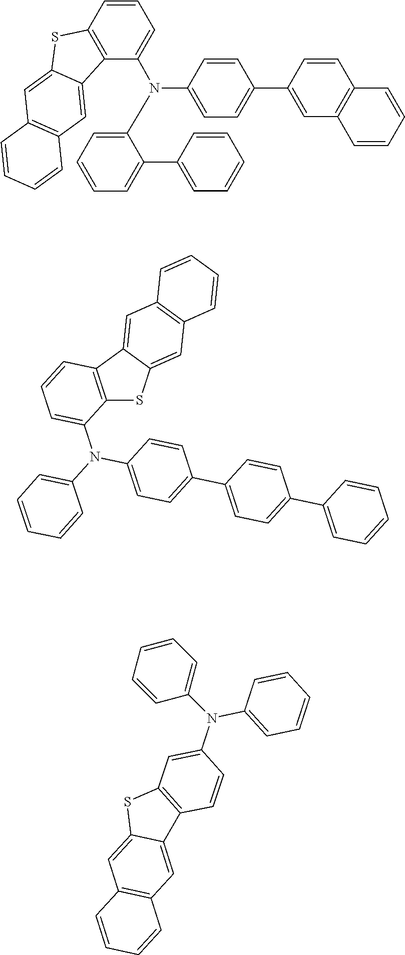 [SMILES: c1ccc(-c2ccccc2N(c2ccc(-c3ccc4ccccc4c3)cc2)c2cccc3sc4cc5ccccc5cc4c23)cc1 ; c1ccc(-c2ccc(-c3ccc(N(c4ccccc4)c4cccc5c4sc4cc6ccccc6cc45)cc3)cc2)cc1 ; c1ccc(N(c2ccccc2)c2ccc3c(c2)sc2cc4ccccc4cc23)cc1] 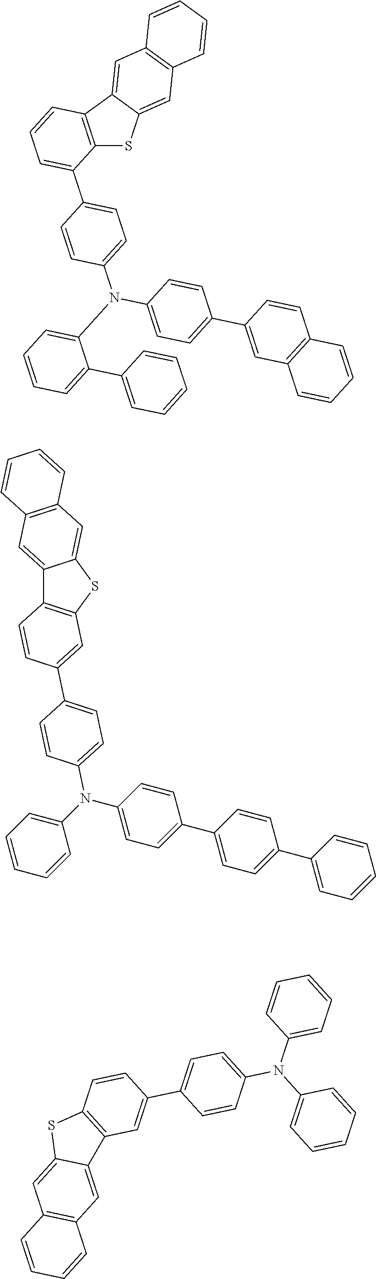 [SMILES: c1ccc(-c2ccccc2N(c2ccc(-c3ccc4ccccc4c3)cc2)c2ccc(-c3cccc4c3sc3cc5ccccc5cc34)cc2)cc1 ; c1ccc(-c2ccc(-c3ccc(N(c4ccccc4)c4ccc(-c5ccc6c(c5)sc5cc7ccccc7cc56)cc4)cc3)cc2)cc1 ; c1ccc(N(c2ccccc2)c2ccc(-c3ccc4sc5cc6ccccc6cc5c4c3)cc2)cc1] 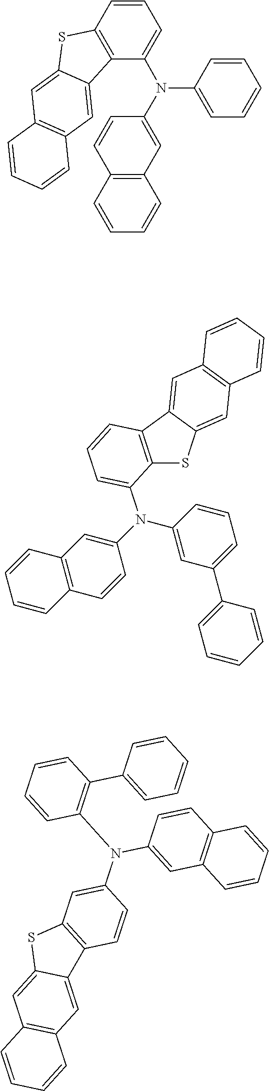 [SMILES: c1ccc(N(c2ccc3ccccc3c2)c2cccc3sc4cc5ccccc5cc4c23)cc1 ; c1ccc(-c2cccc(N(c3ccc4ccccc4c3)c3cccc4c3sc3cc5ccccc5cc34)c2)cc1 ; c1ccc(-c2ccccc2N(c2ccc3ccccc3c2)c2ccc3c(c2)sc2cc4ccccc4cc23)cc1] 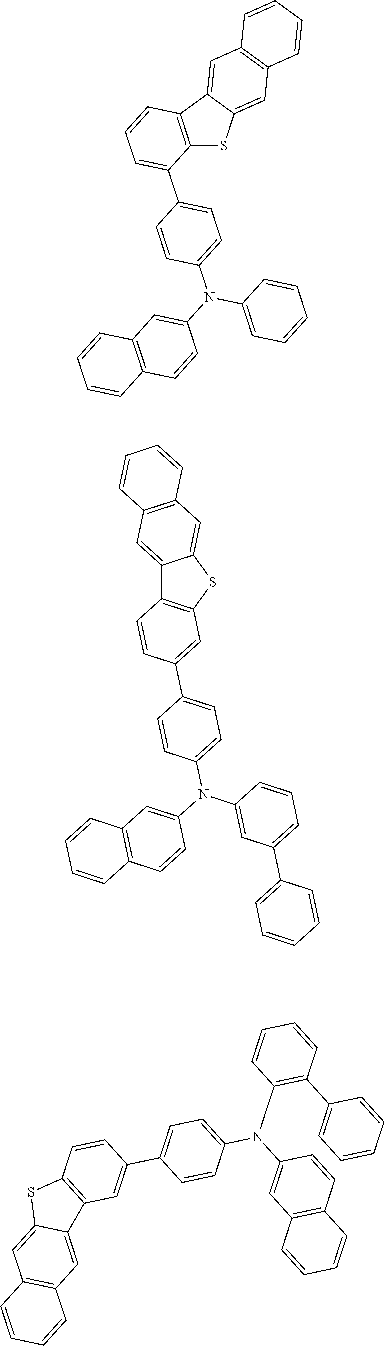 [SMILES: c1ccc(N(c2ccc(-c3cccc4c3sc3cc5ccccc5cc34)cc2)c2ccc3ccccc3c2)cc1 ; c1ccc(-c2cccc(N(c3ccc(-c4ccc5c(c4)sc4cc6ccccc6cc45)cc3)c3ccc4ccccc4c3)c2)cc1 ; c1ccc(-c2ccccc2N(c2ccc(-c3ccc4sc5cc6ccccc6cc5c4c3)cc2)c2ccc3ccccc3c2)cc1] 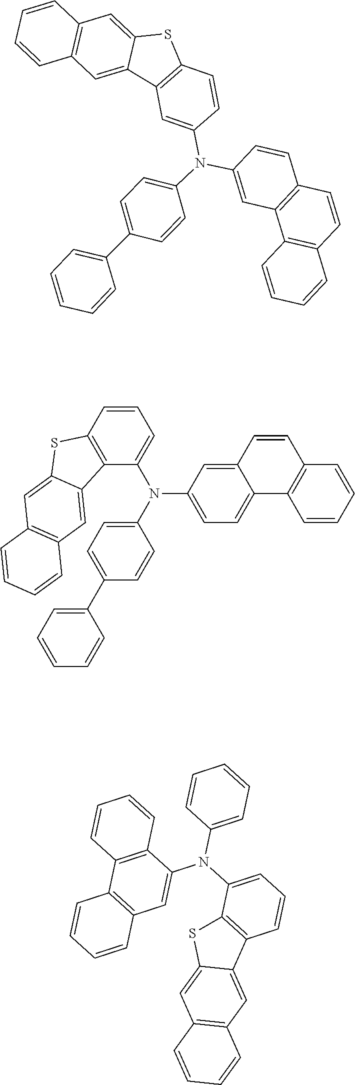 [SMILES: c1ccc(-c2ccc(N(c3ccc4ccc5ccccc5c4c3)c3ccc4sc5cc6ccccc6cc5c4c3)cc2)cc1 ; c1ccc(-c2ccc(N(c3ccc4c(ccc5ccccc54)c3)c3cccc4sc5cc6ccccc6cc5c34)cc2)cc1 ; c1ccc(N(c2cc3ccccc3c3ccccc23)c2cccc3c2sc2cc4ccccc4cc23)cc1] 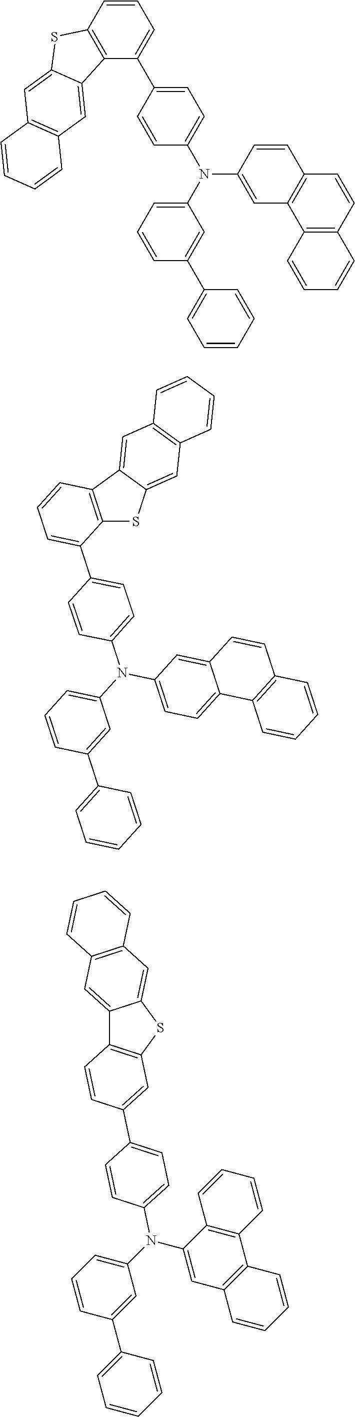 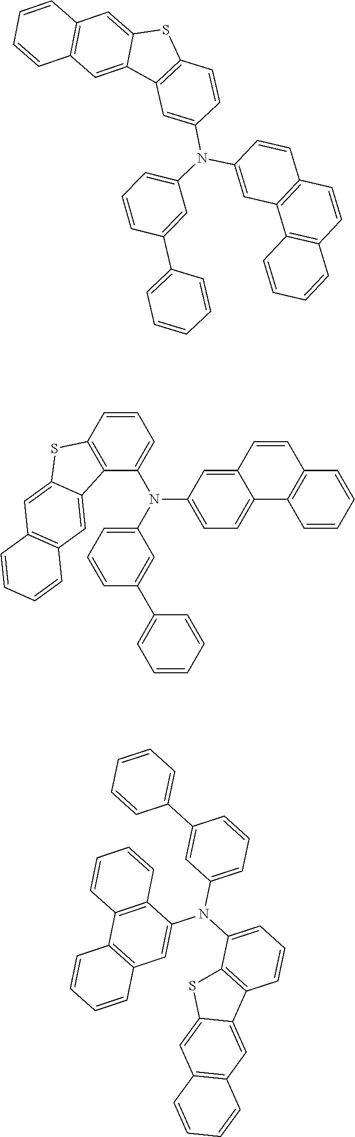 [SMILES: c1ccc(-c2cccc(N(c3ccc4ccc5ccccc5c4c3)c3ccc4sc5cc6ccccc6cc5c4c3)c2)cc1 ; c1ccc(-c2cccc(N(c3ccc4c(ccc5ccccc54)c3)c3cccc4sc5cc6ccccc6cc5c34)c2)cc1 ; c1ccc(-c2cccc(N(c3cc4ccccc4c4ccccc34)c3cccc4c3sc3cc5ccccc5cc34)c2)cc1] 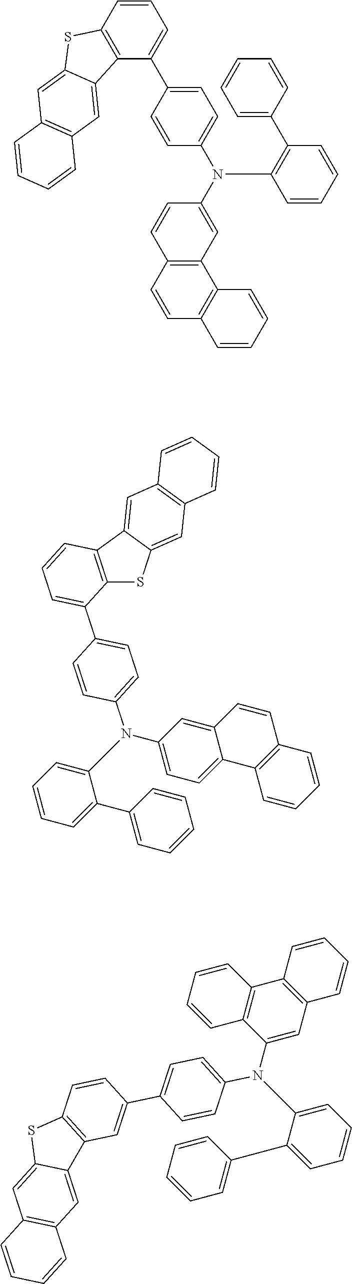 [SMILES: c1ccc(-c2ccccc2N(c2ccc(-c3cccc4sc5cc6ccccc6cc5c34)cc2)c2ccc3ccc4ccccc4c3c2)cc1 ; c1ccc(-c2ccccc2N(c2ccc(-c3cccc4c3sc3cc5ccccc5cc34)cc2)c2ccc3c(ccc4ccccc43)c2)cc1 ; c1ccc(-c2ccccc2N(c2ccc(-c3ccc4sc5cc6ccccc6cc5c4c3)cc2)c2cc3ccccc3c3ccccc23)cc1] 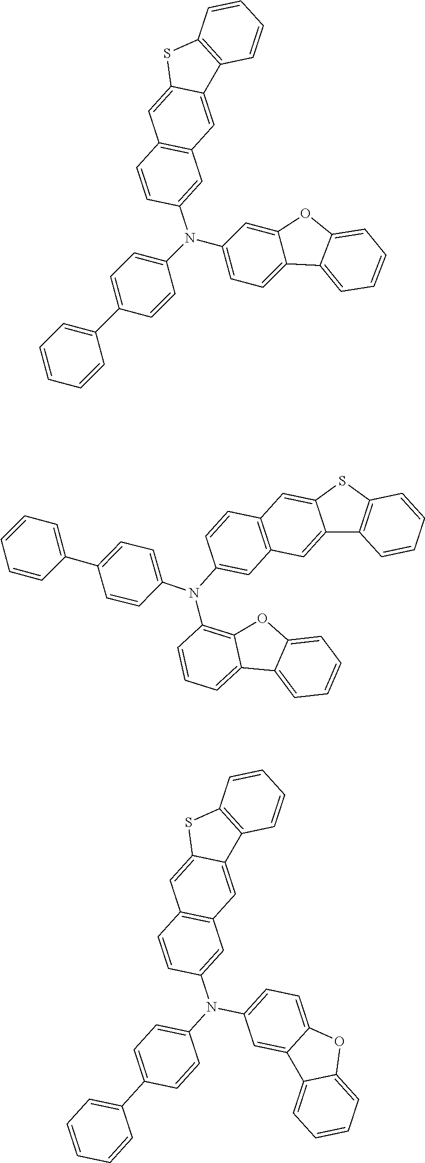 [SMILES: c1ccc(-c2ccc(N(c3ccc4cc5sc6ccccc6c5cc4c3)c3ccc4c(c3)oc3ccccc34)cc2)cc1 ; c1ccc(-c2ccc(N(c3ccc4cc5sc6ccccc6c5cc4c3)c3cccc4c3oc3ccccc34)cc2)cc1 ; c1ccc(-c2ccc(N(c3ccc4cc5sc6ccccc6c5cc4c3)c3ccc4oc5ccccc5c4c3)cc2)cc1] 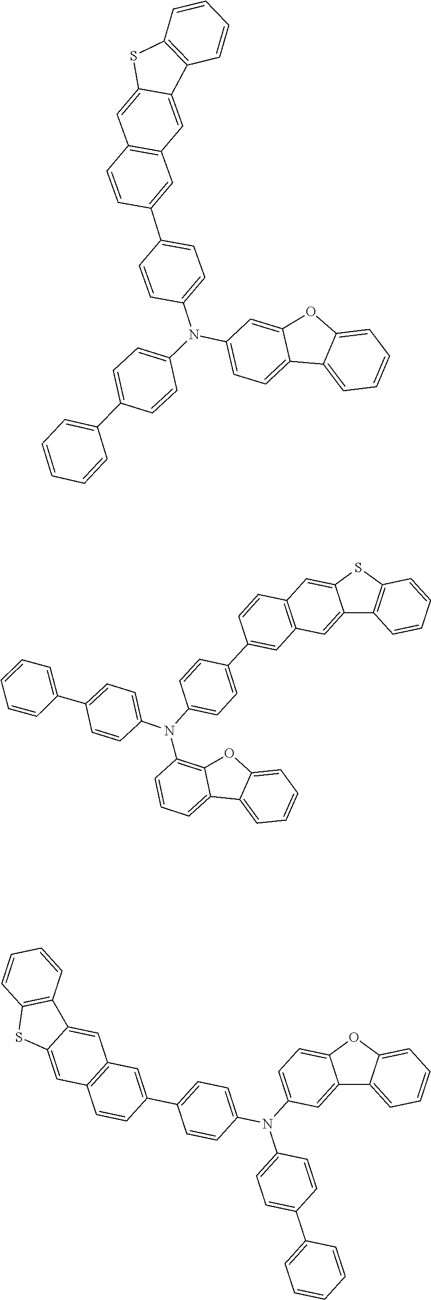 [SMILES: c1ccc(-c2ccc(N(c3ccc(-c4ccc5cc6sc7ccccc7c6cc5c4)cc3)c3ccc4c(c3)oc3ccccc34)cc2)cc1 ; c1ccc(-c2ccc(N(c3ccc(-c4ccc5cc6sc7ccccc7c6cc5c4)cc3)c3cccc4c3oc3ccccc34)cc2)cc1 ; c1ccc(-c2ccc(N(c3ccc(-c4ccc5cc6sc7ccccc7c6cc5c4)cc3)c3ccc4oc5ccccc5c4c3)cc2)cc1] 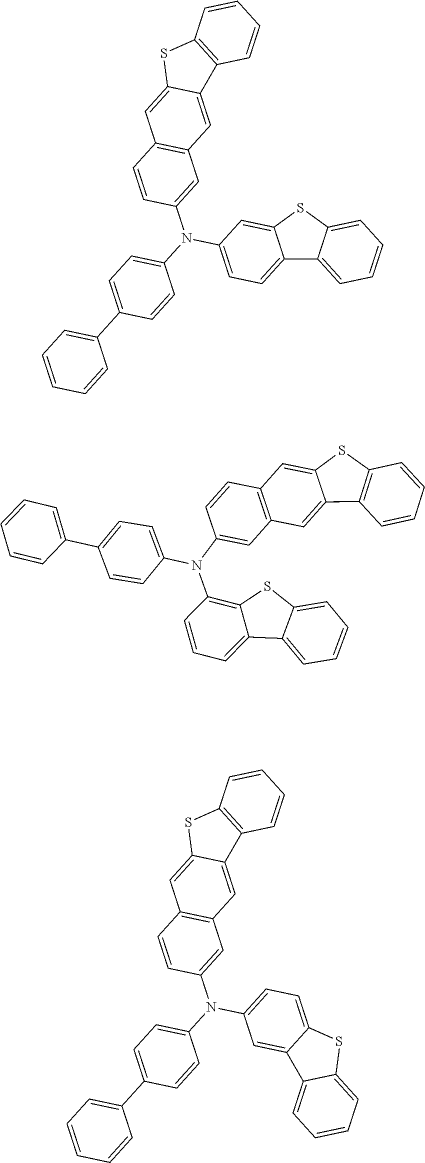 [SMILES: c1ccc(-c2ccc(N(c3ccc4cc5sc6ccccc6c5cc4c3)c3ccc4c(c3)sc3ccccc34)cc2)cc1 ; c1ccc(-c2ccc(N(c3ccc4cc5sc6ccccc6c5cc4c3)c3cccc4c3sc3ccccc34)cc2)cc1 ; c1ccc(-c2ccc(N(c3ccc4cc5sc6ccccc6c5cc4c3)c3ccc4sc5ccccc5c4c3)cc2)cc1] 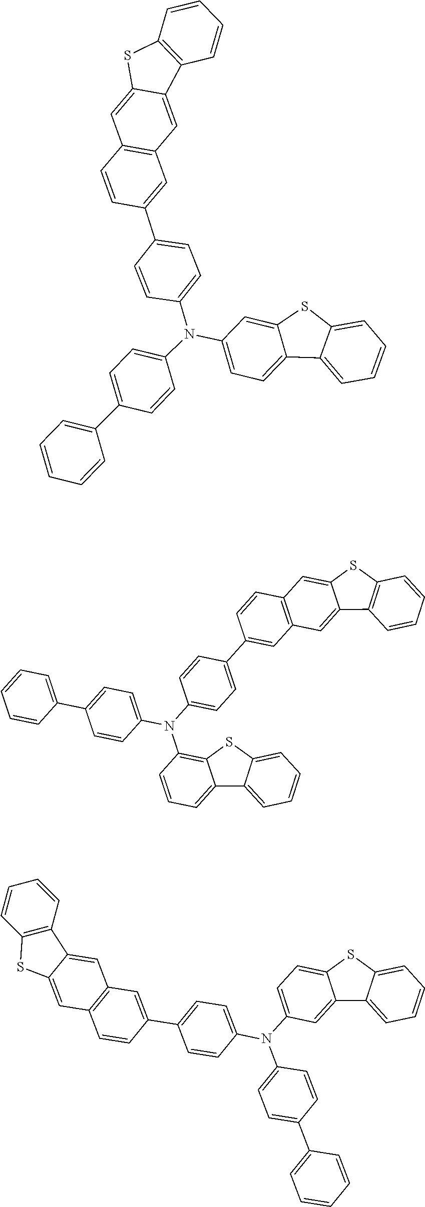 [SMILES: c1ccc(-c2ccc(N(c3ccc(-c4ccc5cc6sc7ccccc7c6cc5c4)cc3)c3ccc4c(c3)sc3ccccc34)cc2)cc1 ; c1ccc(-c2ccc(N(c3ccc(-c4ccc5cc6sc7ccccc7c6cc5c4)cc3)c3cccc4c3sc3ccccc34)cc2)cc1 ; c1ccc(-c2ccc(N(c3ccc(-c4ccc5cc6sc7ccccc7c6cc5c4)cc3)c3ccc4sc5ccccc5c4c3)cc2)cc1] 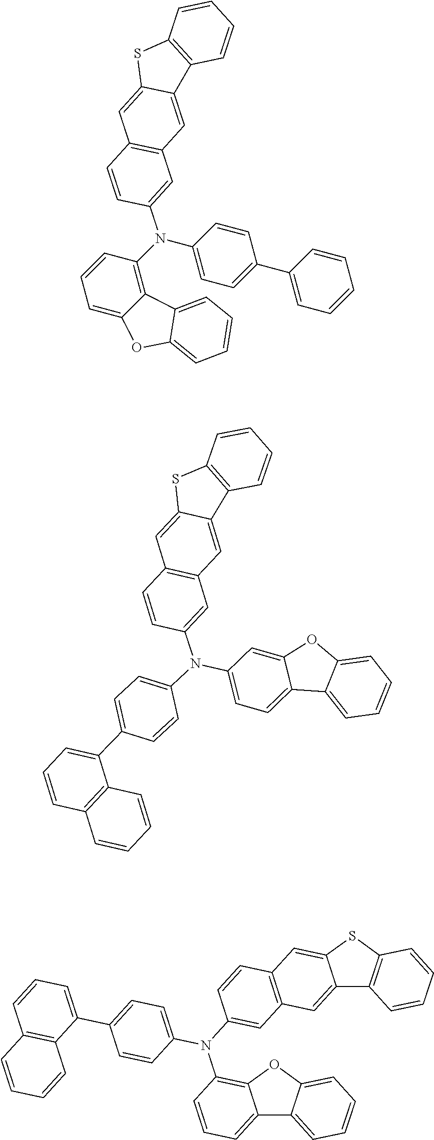 [SMILES: c1ccc(-c2ccc(N(c3ccc4cc5sc6ccccc6c5cc4c3)c3cccc4oc5ccccc5c34)cc2)cc1 ; c1ccc2c(-c3ccc(N(c4ccc5cc6sc7ccccc7c6cc5c4)c4ccc5c(c4)oc4ccccc45)cc3)cccc2c1 ; c1ccc2c(-c3ccc(N(c4ccc5cc6sc7ccccc7c6cc5c4)c4cccc5c4oc4ccccc45)cc3)cccc2c1] 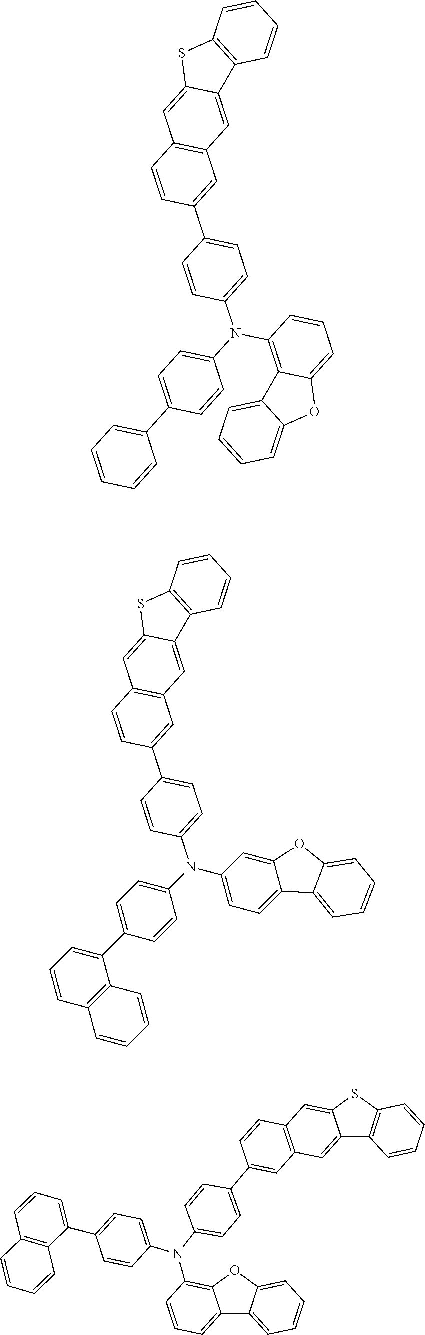 [SMILES: c1ccc(-c2ccc(N(c3ccc(-c4ccc5cc6sc7ccccc7c6cc5c4)cc3)c3cccc4oc5ccccc5c34)cc2)cc1 ; c1ccc2c(-c3ccc(N(c4ccc(-c5ccc6cc7sc8ccccc8c7cc6c5)cc4)c4ccc5c(c4)oc4ccccc45)cc3)cccc2c1 ; c1ccc2c(-c3ccc(N(c4ccc(-c5ccc6cc7sc8ccccc8c7cc6c5)cc4)c4cccc5c4oc4ccccc45)cc3)cccc2c1] 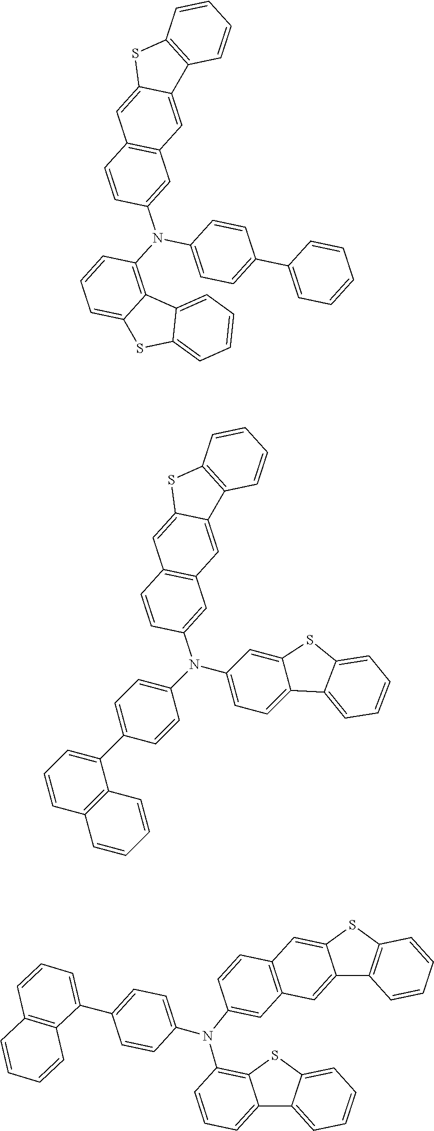 [SMILES: c1ccc(-c2ccc(N(c3ccc4cc5sc6ccccc6c5cc4c3)c3cccc4sc5ccccc5c34)cc2)cc1 ; c1ccc2c(-c3ccc(N(c4ccc5cc6sc7ccccc7c6cc5c4)c4ccc5c(c4)sc4ccccc45)cc3)cccc2c1 ; c1ccc2c(-c3ccc(N(c4ccc5cc6sc7ccccc7c6cc5c4)c4cccc5c4sc4ccccc45)cc3)cccc2c1] 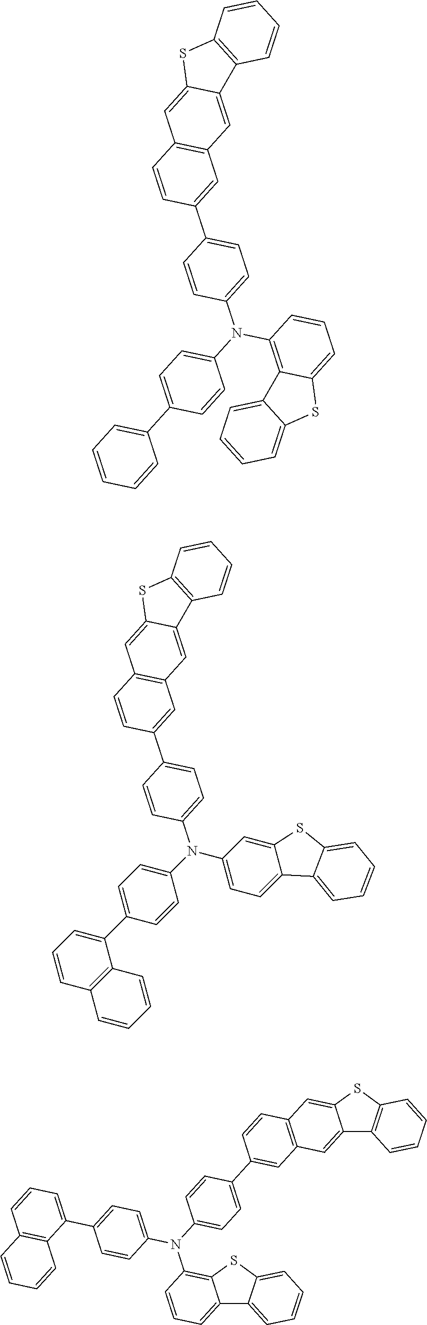 [SMILES: c1ccc(-c2ccc(N(c3ccc(-c4ccc5cc6sc7ccccc7c6cc5c4)cc3)c3cccc4sc5ccccc5c34)cc2)cc1 ; c1ccc2c(-c3ccc(N(c4ccc(-c5ccc6cc7sc8ccccc8c7cc6c5)cc4)c4ccc5c(c4)sc4ccccc45)cc3)cccc2c1 ; c1ccc2c(-c3ccc(N(c4ccc(-c5ccc6cc7sc8ccccc8c7cc6c5)cc4)c4cccc5c4sc4ccccc45)cc3)cccc2c1] 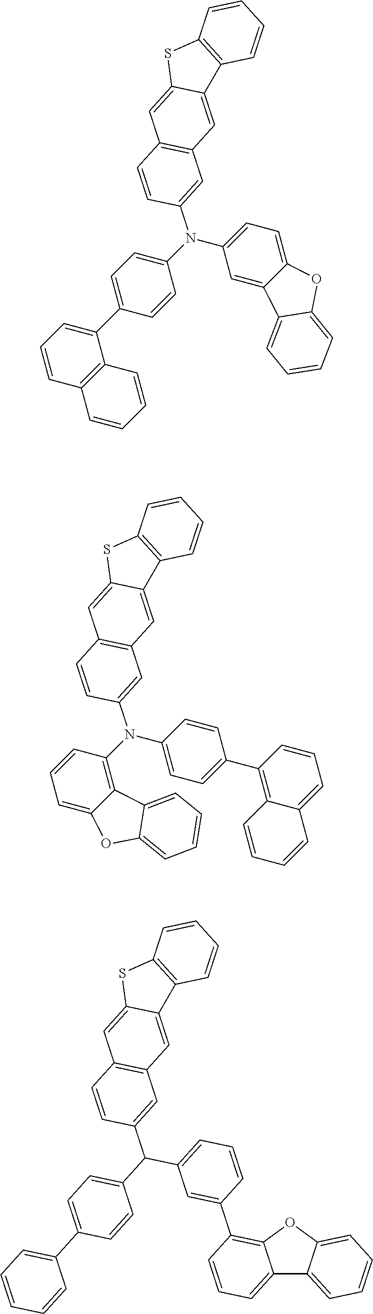 [SMILES: c1ccc2c(-c3ccc(N(c4ccc5cc6sc7ccccc7c6cc5c4)c4ccc5oc6ccccc6c5c4)cc3)cccc2c1 ; c1ccc2c(-c3ccc(N(c4ccc5cc6sc7ccccc7c6cc5c4)c4cccc5oc6ccccc6c45)cc3)cccc2c1 ; c1ccc(-c2ccc(C(c3cccc(-c4cccc5c4oc4ccccc45)c3)c3ccc4cc5sc6ccccc6c5cc4c3)cc2)cc1] 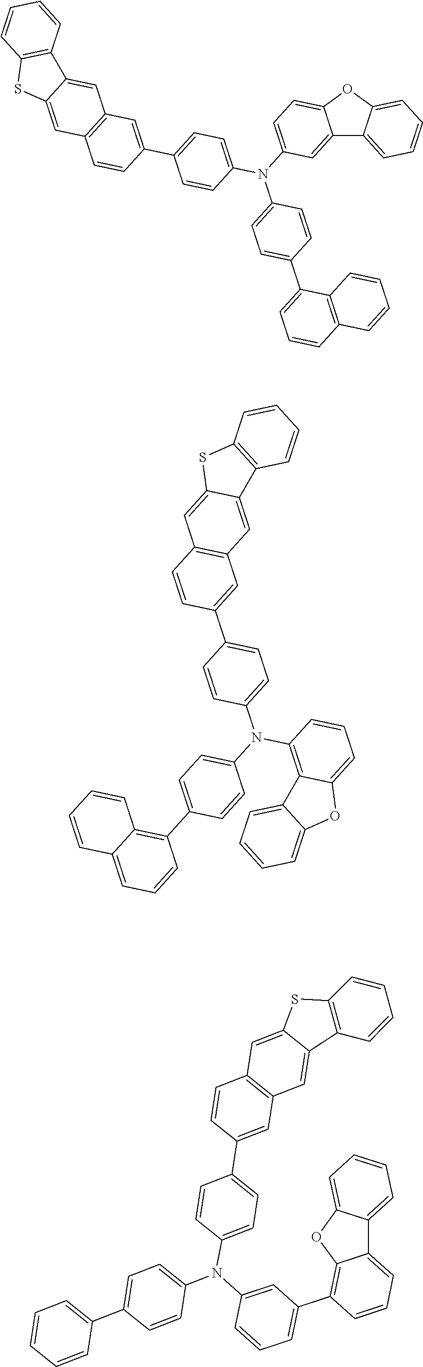 [SMILES: c1ccc2c(-c3ccc(N(c4ccc(-c5ccc6cc7sc8ccccc8c7cc6c5)cc4)c4ccc5oc6ccccc6c5c4)cc3)cccc2c1 ; c1ccc2c(-c3ccc(N(c4ccc(-c5ccc6cc7sc8ccccc8c7cc6c5)cc4)c4cccc5oc6ccccc6c45)cc3)cccc2c1 ; c1ccc(-c2ccc(N(c3ccc(-c4ccc5cc6sc7ccccc7c6cc5c4)cc3)c3cccc(-c4cccc5c4oc4ccccc45)c3)cc2)cc1] 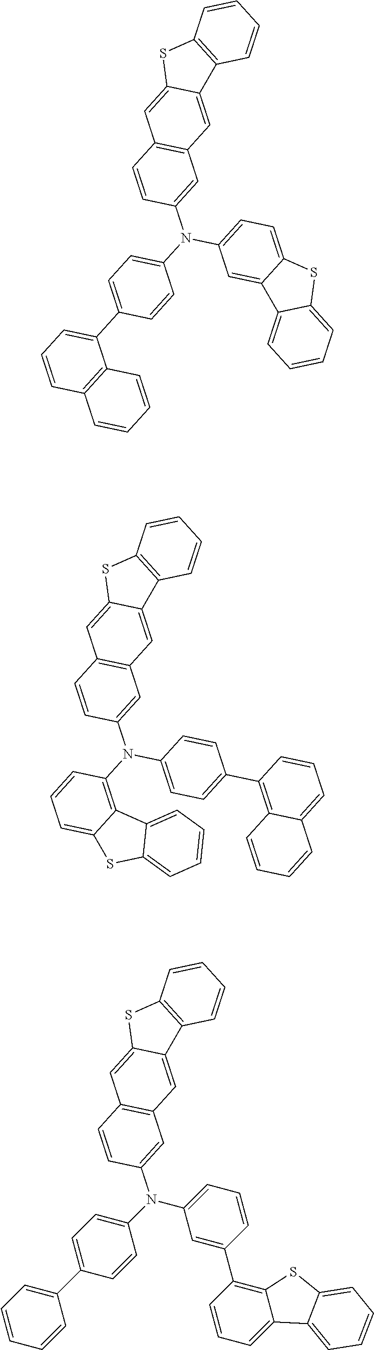 [SMILES: c1ccc2c(-c3ccc(N(c4ccc5cc6sc7ccccc7c6cc5c4)c4ccc5sc6ccccc6c5c4)cc3)cccc2c1 ; c1ccc2c(-c3ccc(N(c4ccc5cc6sc7ccccc7c6cc5c4)c4cccc5sc6ccccc6c45)cc3)cccc2c1 ; c1ccc(-c2ccc(N(c3cccc(-c4cccc5c4sc4ccccc45)c3)c3ccc4cc5sc6ccccc6c5cc4c3)cc2)cc1] 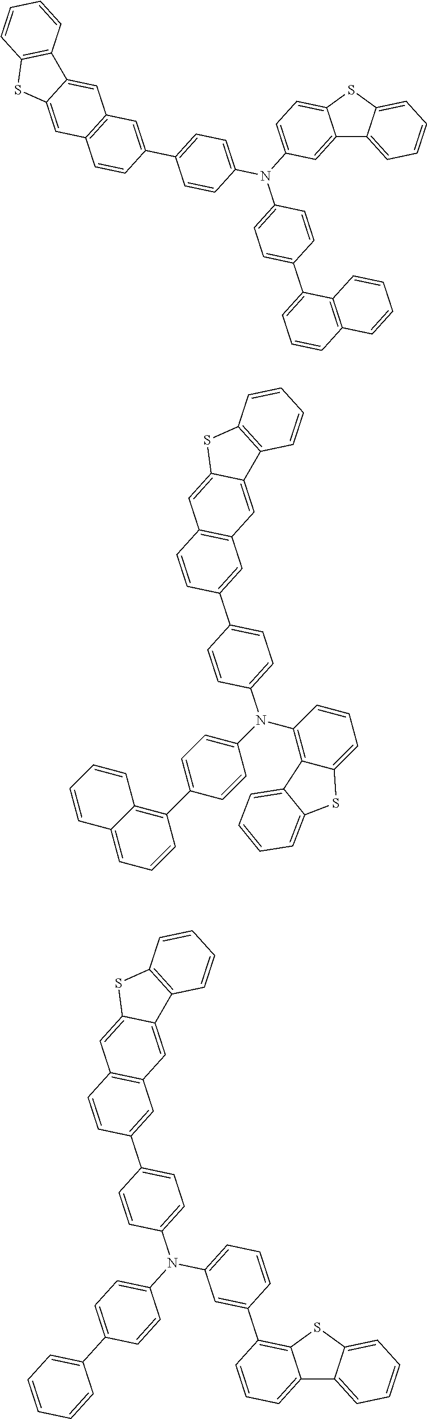 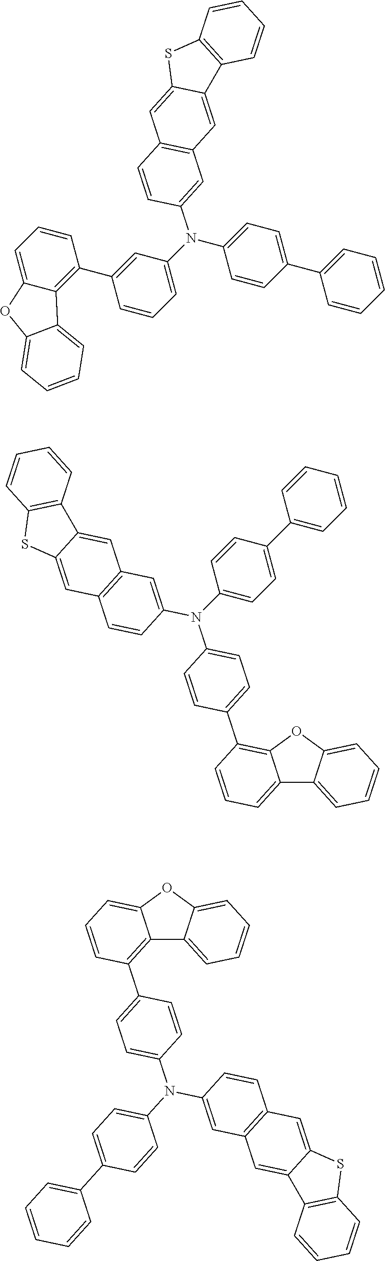 [SMILES: c1ccc(-c2ccc(N(c3cccc(-c4cccc5oc6ccccc6c45)c3)c3ccc4cc5sc6ccccc6c5cc4c3)cc2)cc1 ; c1ccc(-c2ccc(N(c3ccc(-c4cccc5c4oc4ccccc45)cc3)c3ccc4cc5sc6ccccc6c5cc4c3)cc2)cc1 ; c1ccc(-c2ccc(N(c3ccc(-c4cccc5oc6ccccc6c45)cc3)c3ccc4cc5sc6ccccc6c5cc4c3)cc2)cc1] 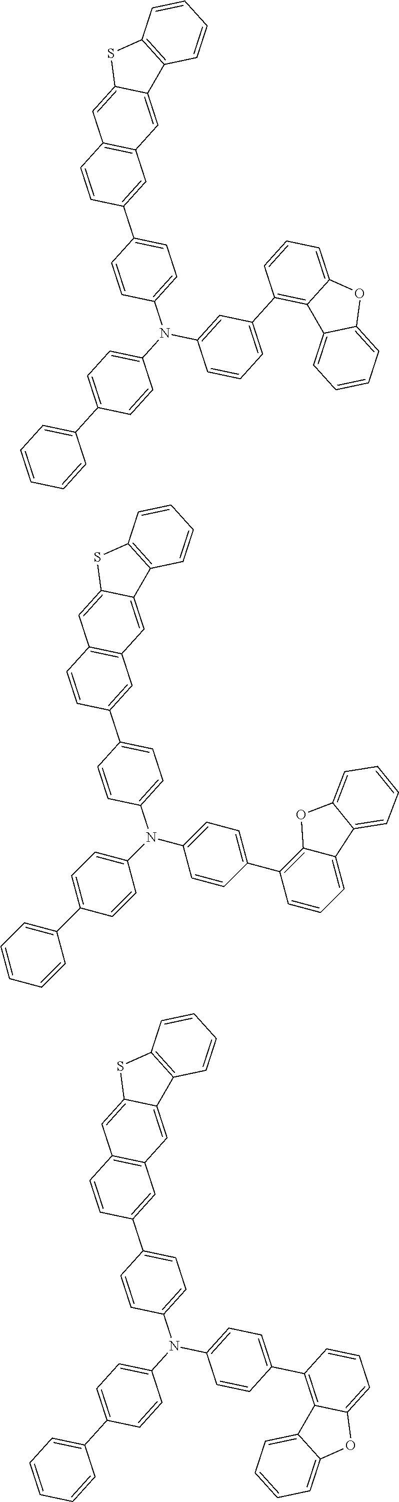 [SMILES: c1ccc(-c2ccc(N(c3ccc(-c4ccc5cc6sc7ccccc7c6cc5c4)cc3)c3cccc(-c4cccc5oc6ccccc6c45)c3)cc2)cc1 ; c1ccc(-c2ccc(N(c3ccc(-c4ccc5cc6sc7ccccc7c6cc5c4)cc3)c3ccc(-c4cccc5c4oc4ccccc45)cc3)cc2)cc1 ; c1ccc(-c2ccc(N(c3ccc(-c4ccc5cc6sc7ccccc7c6cc5c4)cc3)c3ccc(-c4cccc5oc6ccccc6c45)cc3)cc2)cc1] 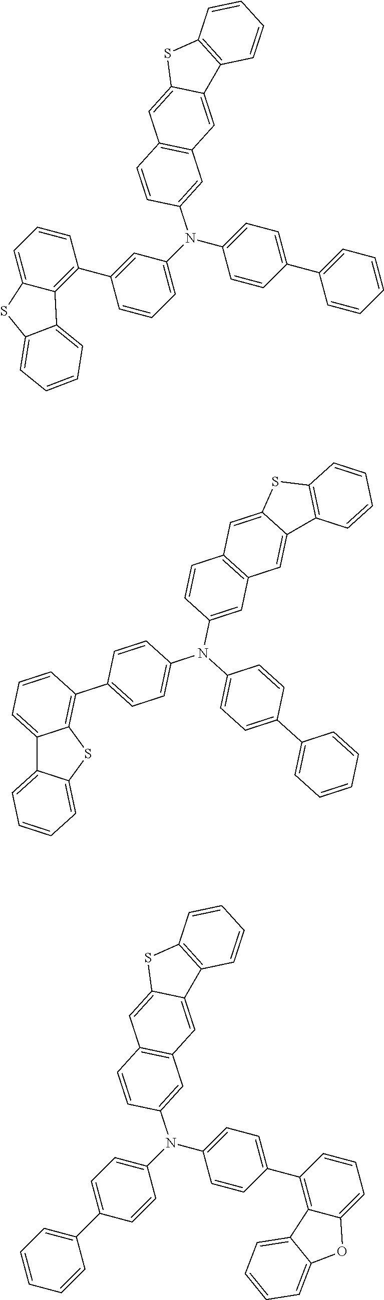 [SMILES: c1ccc(-c2ccc(N(c3cccc(-c4cccc5sc6ccccc6c45)c3)c3ccc4cc5sc6ccccc6c5cc4c3)cc2)cc1 ; c1ccc(-c2ccc(N(c3ccc(-c4cccc5c4sc4ccccc45)cc3)c3ccc4cc5sc6ccccc6c5cc4c3)cc2)cc1 ; c1ccc(-c2ccc(N(c3ccc(-c4cccc5oc6ccccc6c45)cc3)c3ccc4cc5sc6ccccc6c5cc4c3)cc2)cc1] 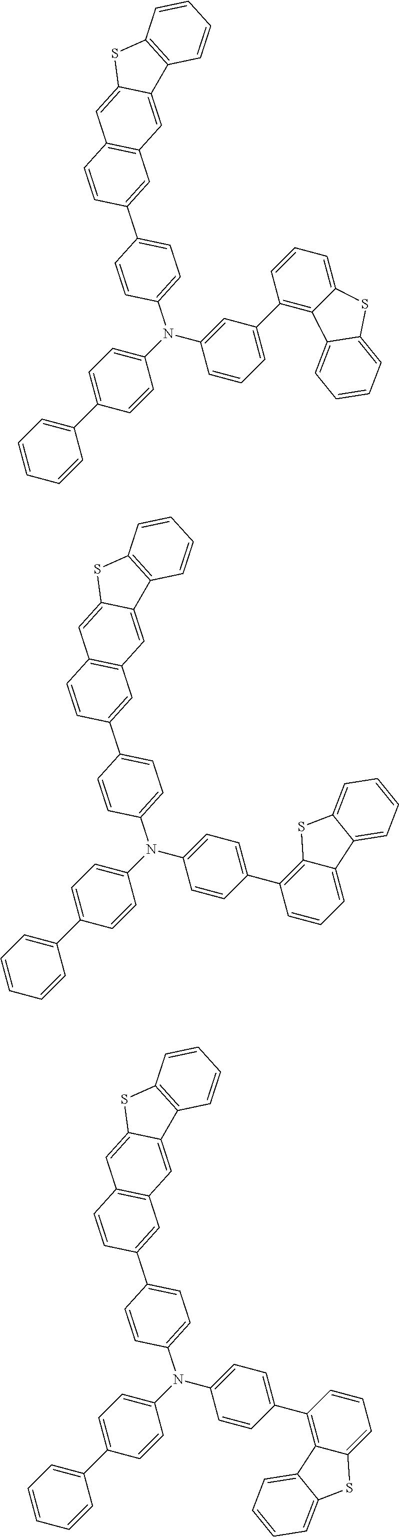 [SMILES: c1ccc(-c2ccc(N(c3ccc(-c4ccc5cc6sc7ccccc7c6cc5c4)cc3)c3cccc(-c4cccc5sc6ccccc6c45)c3)cc2)cc1 ; c1ccc(-c2ccc(N(c3ccc(-c4ccc5cc6sc7ccccc7c6cc5c4)cc3)c3ccc(-c4cccc5c4sc4ccccc45)cc3)cc2)cc1 ; c1ccc(-c2ccc(N(c3ccc(-c4ccc5cc6sc7ccccc7c6cc5c4)cc3)c3ccc(-c4cccc5sc6ccccc6c45)cc3)cc2)cc1] 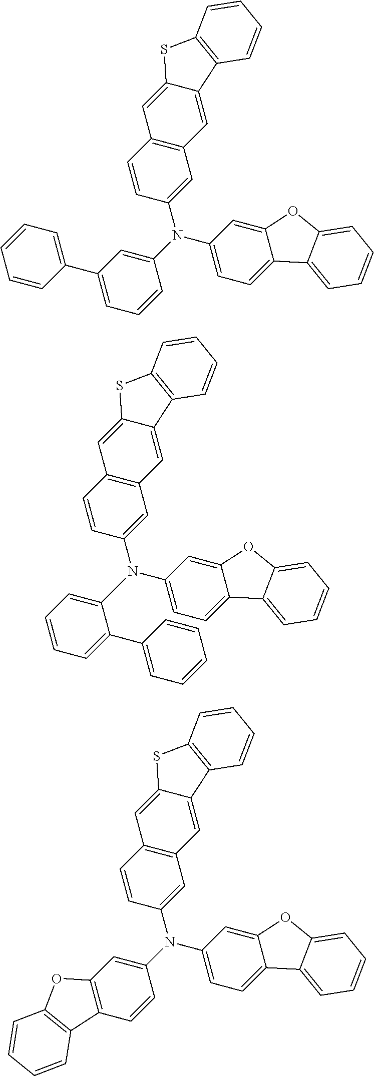 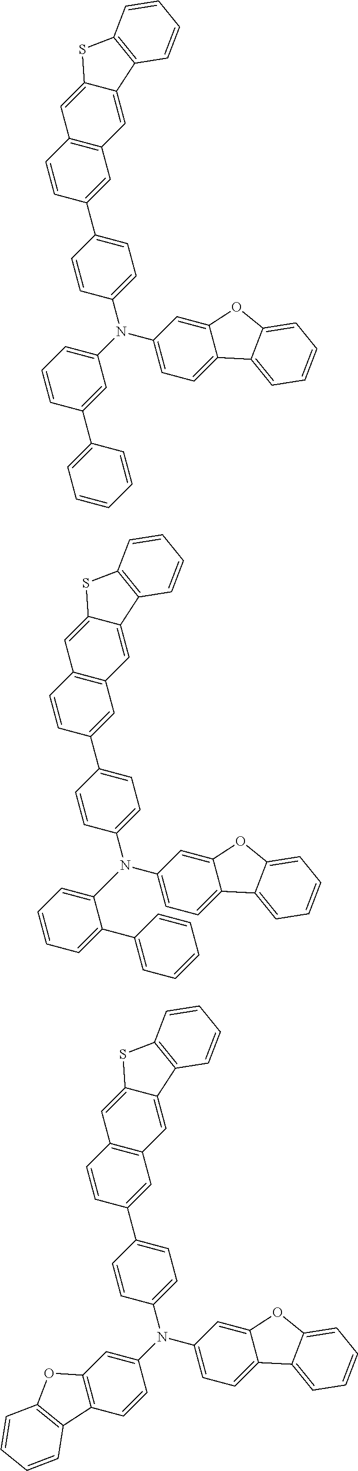 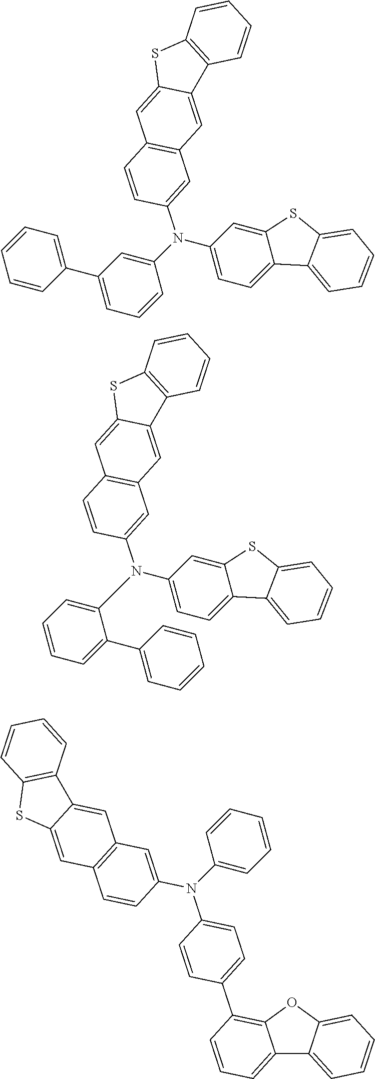 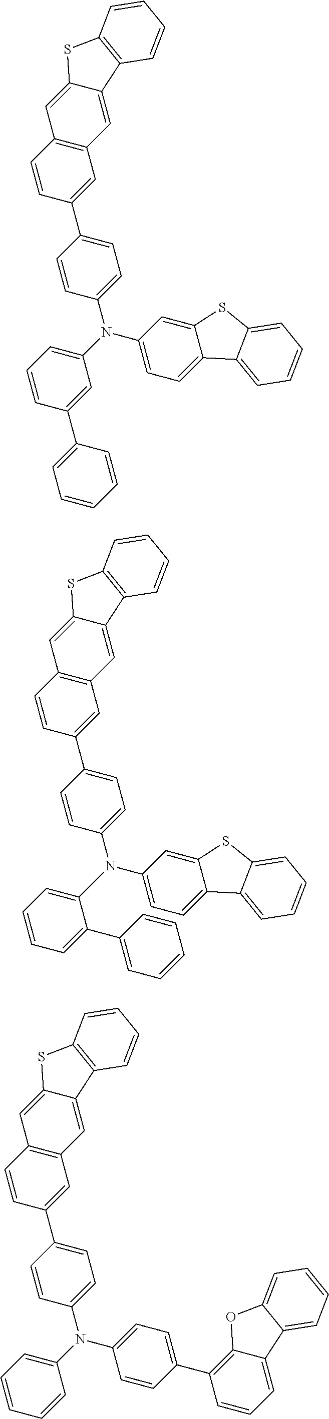 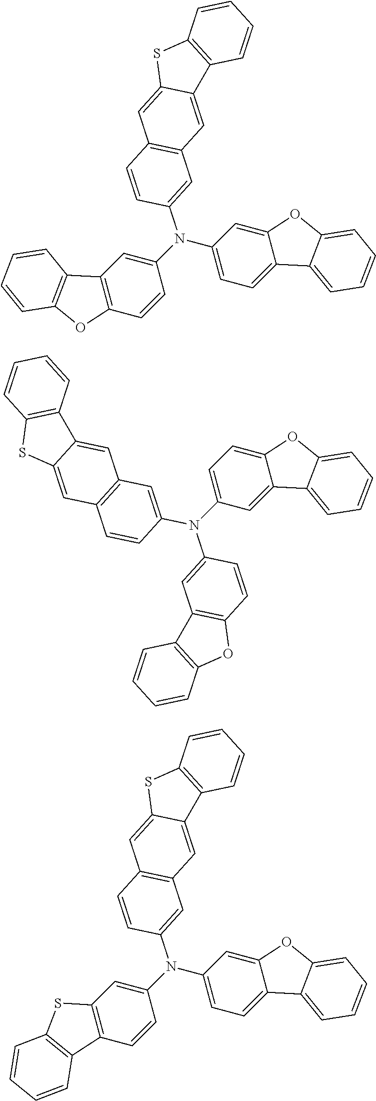 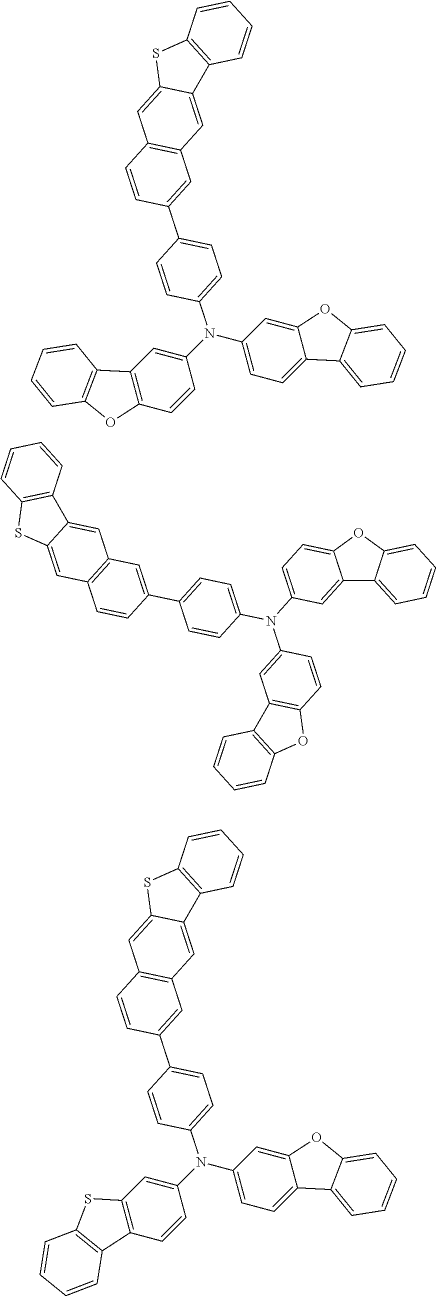 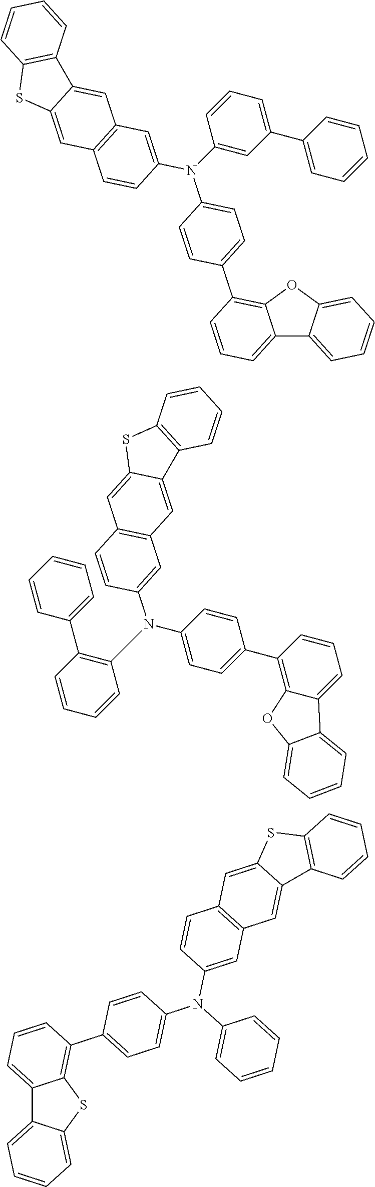 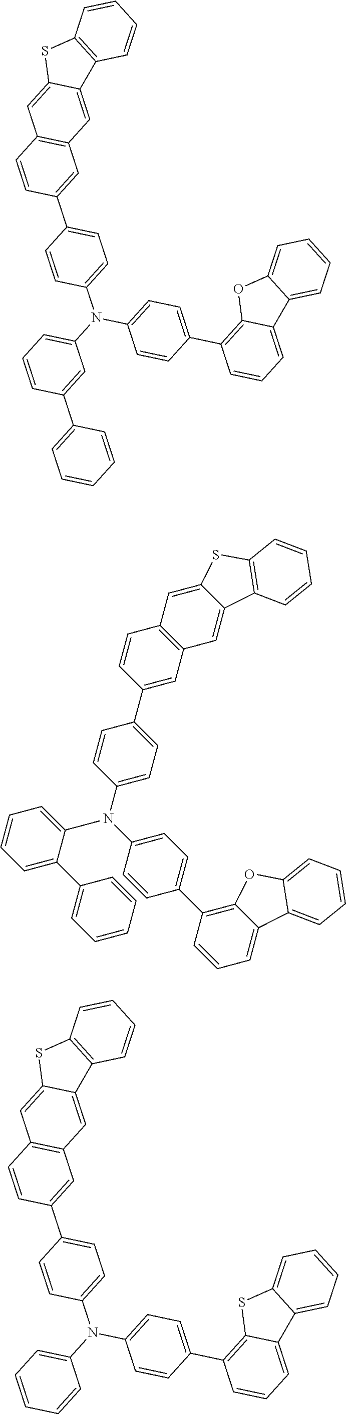 [SMILES: c1ccc(-c2cccc(N(c3ccc(-c4ccc5cc6sc7ccccc7c6cc5c4)cc3)c3ccc(-c4cccc5c4oc4ccccc45)cc3)c2)cc1 ; c1ccc(-c2ccccc2N(c2ccc(-c3ccc4cc5sc6ccccc6c5cc4c3)cc2)c2ccc(-c3cccc4c3oc3ccccc34)cc2)cc1 ; c1ccc(N(c2ccc(-c3ccc4cc5sc6ccccc6c5cc4c3)cc2)c2ccc(-c3cccc4c3sc3ccccc34)cc2)cc1] 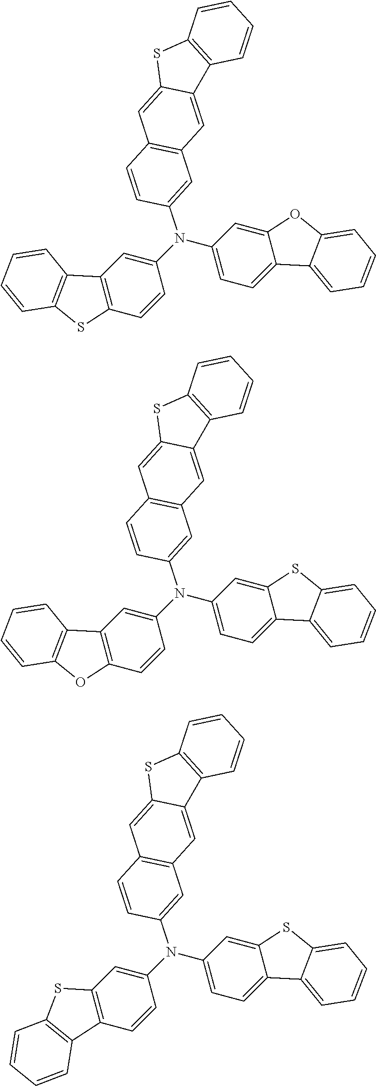 [SMILES: c1ccc2c(c1)oc1cc(N(c3ccc4cc5sc6ccccc6c5cc4c3)c3ccc4sc5ccccc5c4c3)ccc12 ; c1ccc2c(c1)oc1ccc(N(c3ccc4cc5sc6ccccc6c5cc4c3)c3ccc4c(c3)sc3ccccc34)cc12 ; c1ccc2c(c1)sc1cc(N(c3ccc4cc5sc6ccccc6c5cc4c3)c3ccc4c(c3)sc3ccccc34)ccc12] 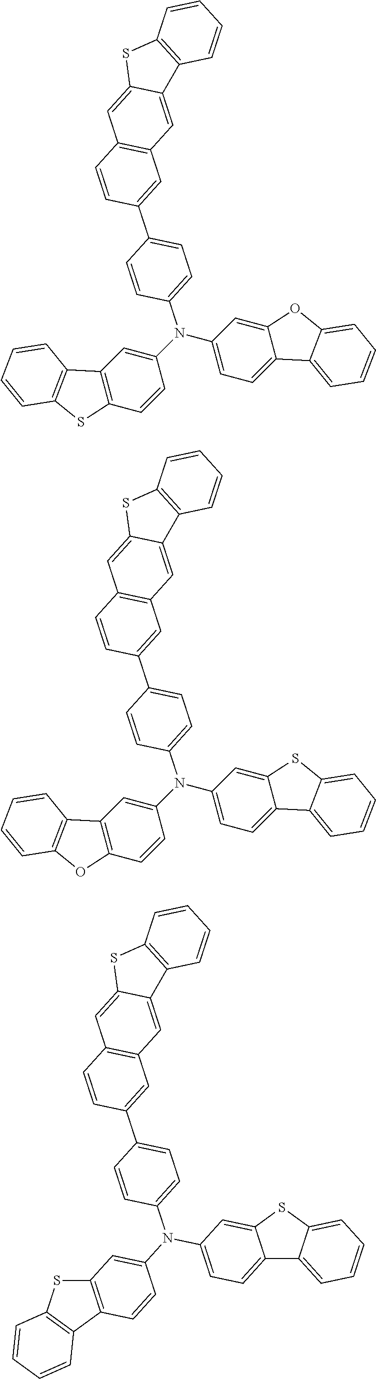 [SMILES: c1ccc2c(c1)oc1cc(N(c3ccc(-c4ccc5cc6sc7ccccc7c6cc5c4)cc3)c3ccc4sc5ccccc5c4c3)ccc12 ; c1ccc2c(c1)oc1ccc(N(c3ccc(-c4ccc5cc6sc7ccccc7c6cc5c4)cc3)c3ccc4c(c3)sc3ccccc34)cc12 ; c1ccc2c(c1)sc1cc(N(c3ccc(-c4ccc5cc6sc7ccccc7c6cc5c4)cc3)c3ccc4c(c3)sc3ccccc34)ccc12] 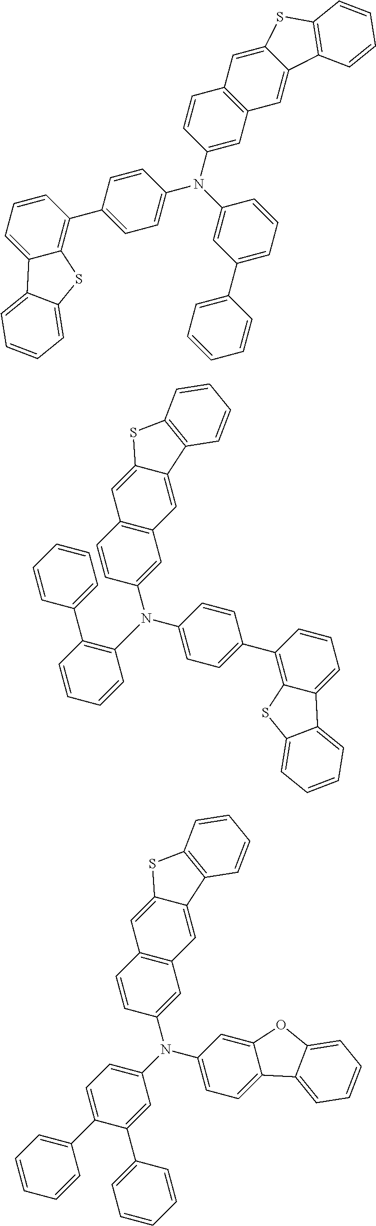 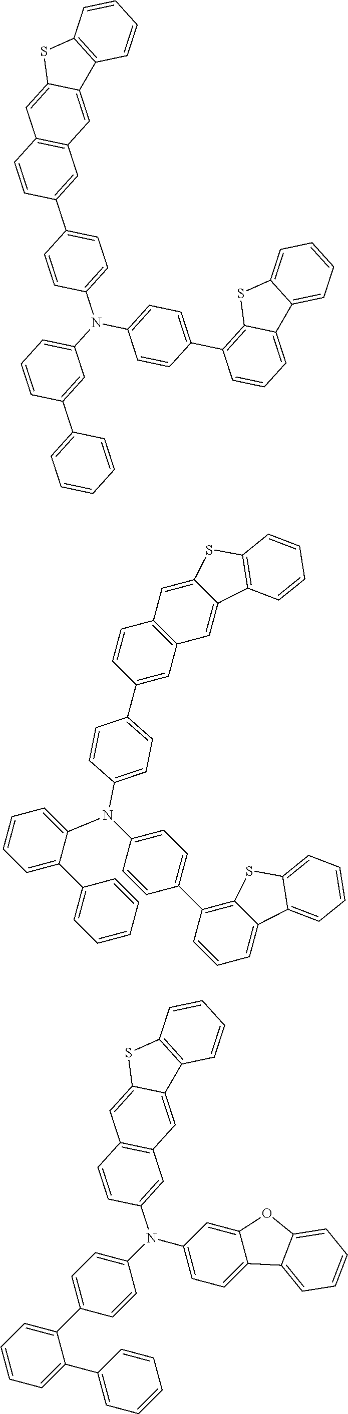 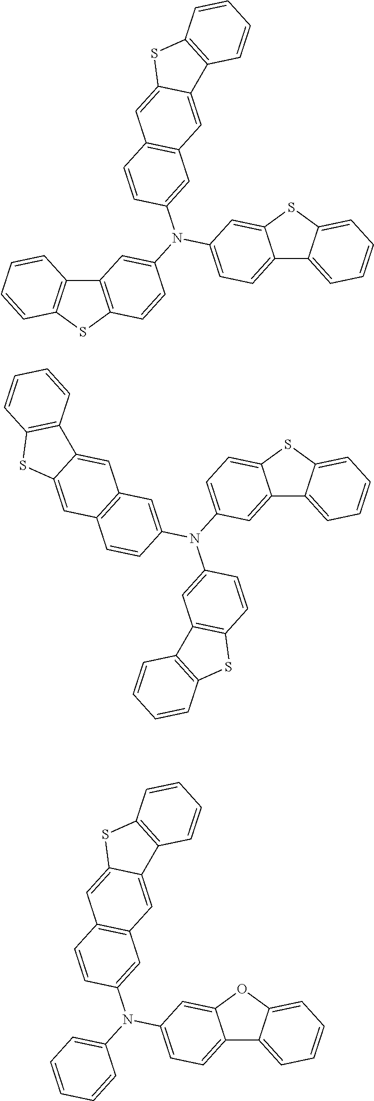 [SMILES: c1ccc2c(c1)sc1cc(N(c3ccc4cc5sc6ccccc6c5cc4c3)c3ccc4sc5ccccc5c4c3)ccc12 ; c1ccc2c(c1)sc1ccc(N(c3ccc4cc5sc6ccccc6c5cc4c3)c3ccc4sc5ccccc5c4c3)cc12 ; c1ccc(N(c2ccc3cc4sc5ccccc5c4cc3c2)c2ccc3c(c2)oc2ccccc23)cc1] 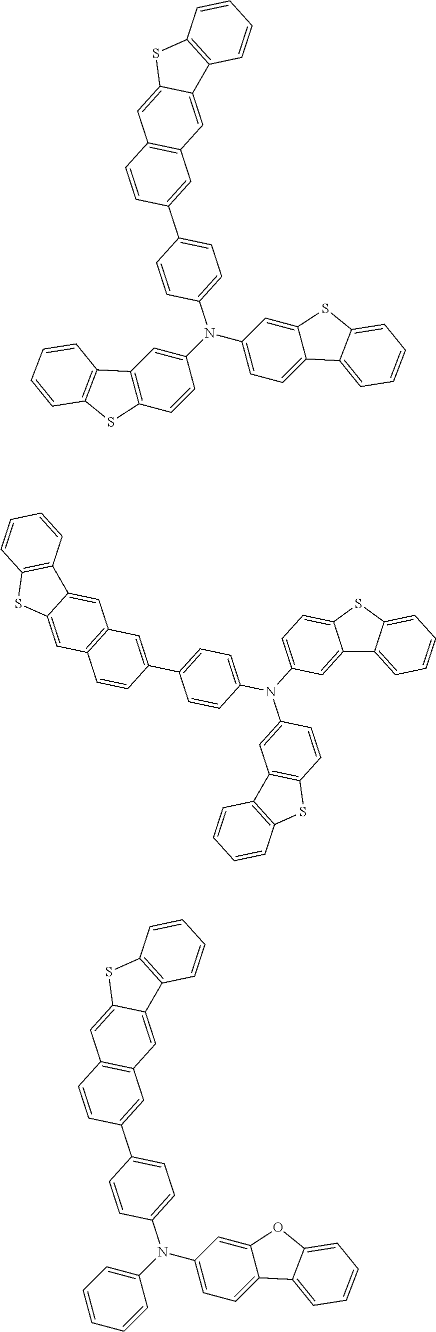 [SMILES: c1ccc2c(c1)sc1cc(N(c3ccc(-c4ccc5cc6sc7ccccc7c6cc5c4)cc3)c3ccc4sc5ccccc5c4c3)ccc12 ; c1ccc2c(c1)sc1ccc(N(c3ccc(-c4ccc5cc6sc7ccccc7c6cc5c4)cc3)c3ccc4sc5ccccc5c4c3)cc12 ; c1ccc(N(c2ccc(-c3ccc4cc5sc6ccccc6c5cc4c3)cc2)c2ccc3c(c2)oc2ccccc23)cc1] 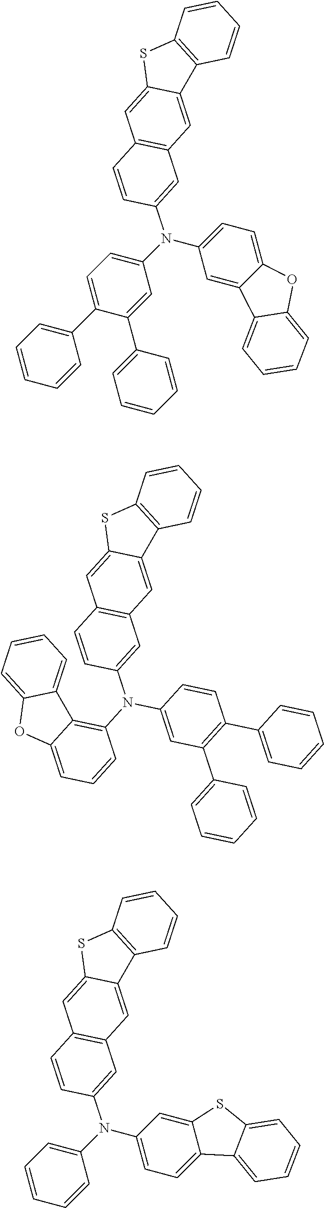 [SMILES: c1ccc(-c2ccc(N(c3ccc4cc5sc6ccccc6c5cc4c3)c3ccc4oc5ccccc5c4c3)cc2-c2ccccc2)cc1 ; c1ccc(-c2ccc(N(c3ccc4cc5sc6ccccc6c5cc4c3)c3cccc4oc5ccccc5c34)cc2-c2ccccc2)cc1 ; c1ccc(N(c2ccc3cc4sc5ccccc5c4cc3c2)c2ccc3c(c2)sc2ccccc23)cc1] 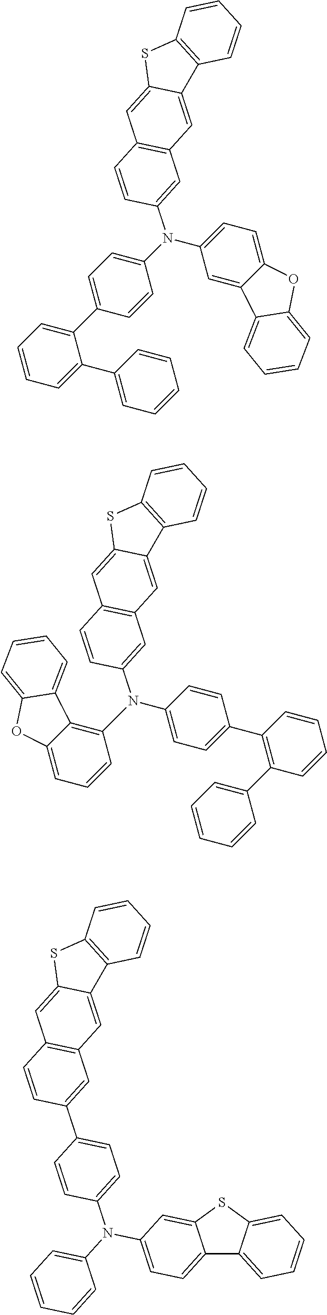 [SMILES: c1ccc(-c2ccccc2-c2ccc(N(c3ccc4cc5sc6ccccc6c5cc4c3)c3ccc4oc5ccccc5c4c3)cc2)cc1 ; c1ccc(-c2ccccc2-c2ccc(N(c3ccc4cc5sc6ccccc6c5cc4c3)c3cccc4oc5ccccc5c34)cc2)cc1 ; c1ccc(N(c2ccc(-c3ccc4cc5sc6ccccc6c5cc4c3)cc2)c2ccc3c(c2)sc2ccccc23)cc1] 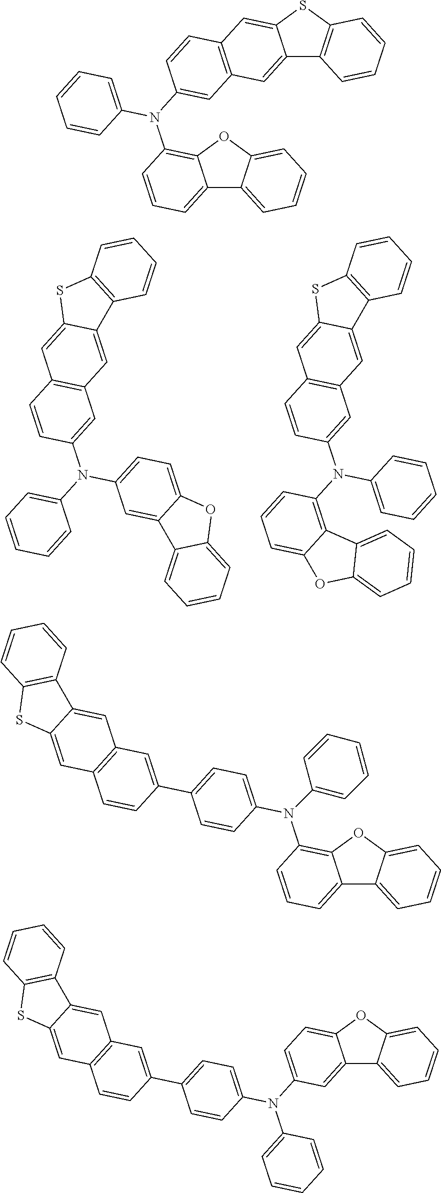 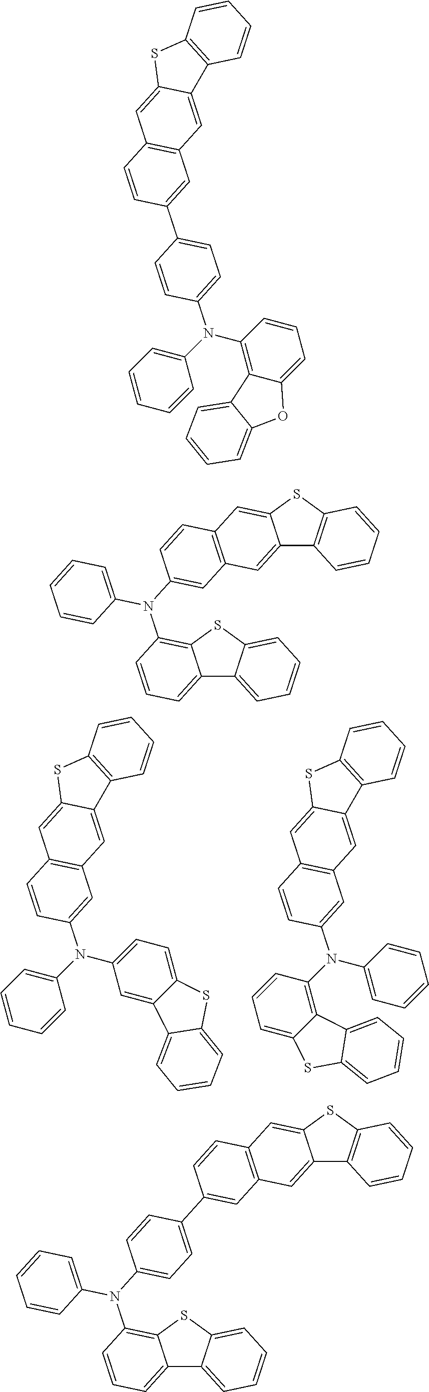 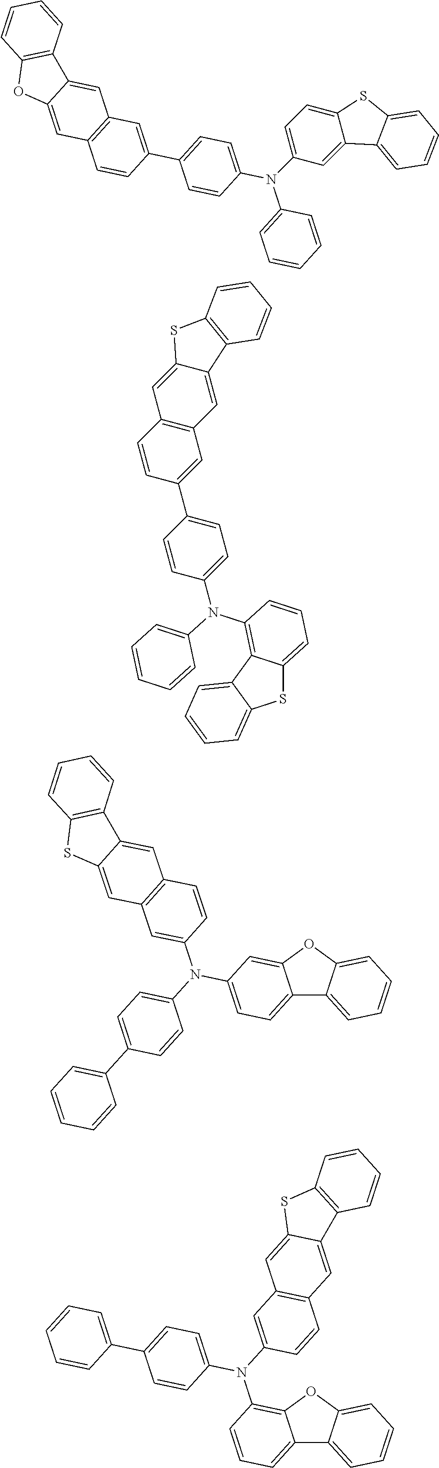 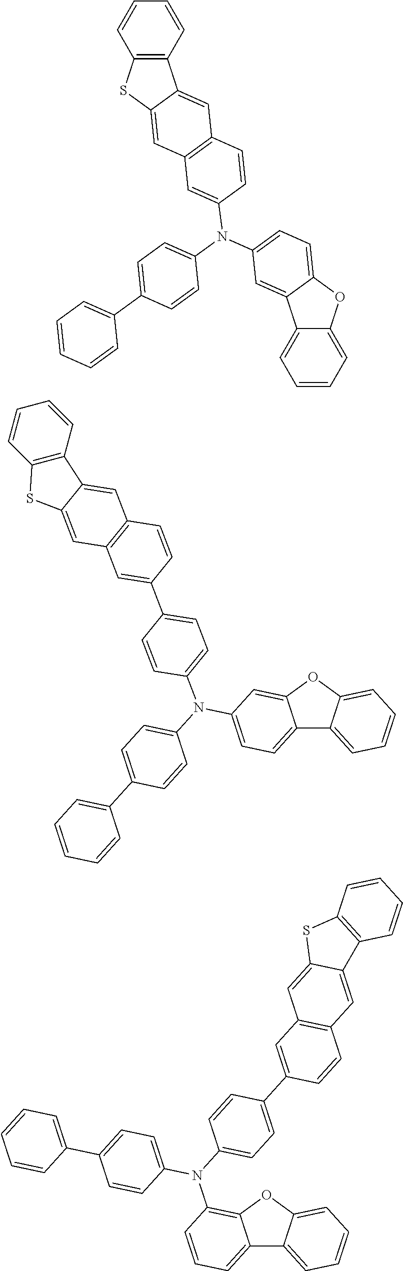 [SMILES: c1ccc(-c2ccc(N(c3ccc4cc5c(cc4c3)sc3ccccc35)c3ccc4oc5ccccc5c4c3)cc2)cc1 ; c1ccc(-c2ccc(N(c3ccc(-c4ccc5cc6c(cc5c4)sc4ccccc46)cc3)c3ccc4c(c3)oc3ccccc34)cc2)cc1 ; c1ccc(-c2ccc(N(c3ccc(-c4ccc5cc6c(cc5c4)sc4ccccc46)cc3)c3cccc4c3oc3ccccc34)cc2)cc1] 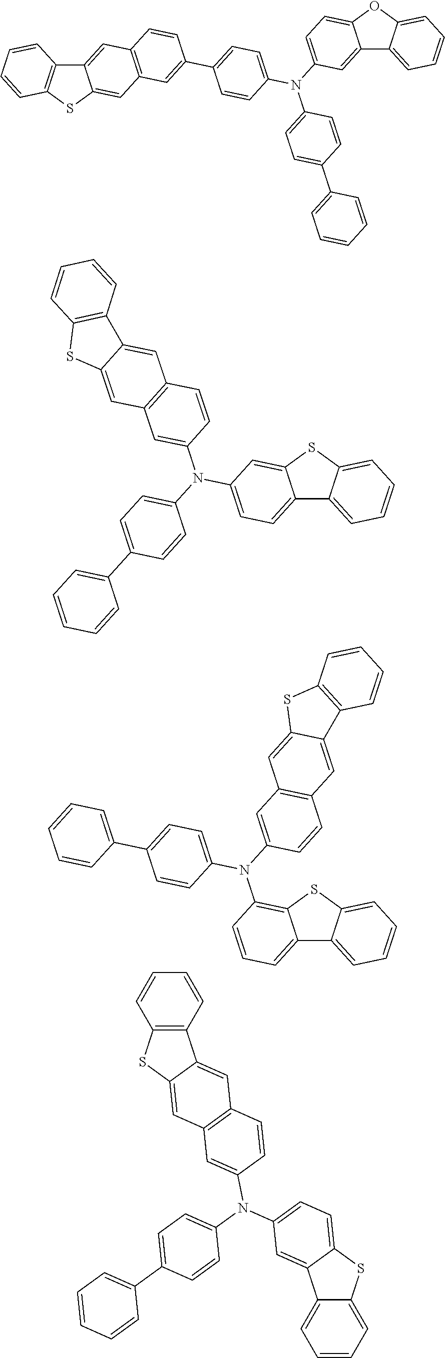 [SMILES: c1ccc(-c2ccc(N(c3ccc(-c4ccc5cc6c(cc5c4)sc4ccccc46)cc3)c3ccc4oc5ccccc5c4c3)cc2)cc1 ; c1ccc(-c2ccc(N(c3ccc4cc5c(cc4c3)sc3ccccc35)c3ccc4c(c3)sc3ccccc34)cc2)cc1 ; c1ccc(-c2ccc(N(c3ccc4cc5c(cc4c3)sc3ccccc35)c3cccc4c3sc3ccccc34)cc2)cc1 ; c1ccc(-c2ccc(N(c3ccc4cc5c(cc4c3)sc3ccccc35)c3ccc4sc5ccccc5c4c3)cc2)cc1] 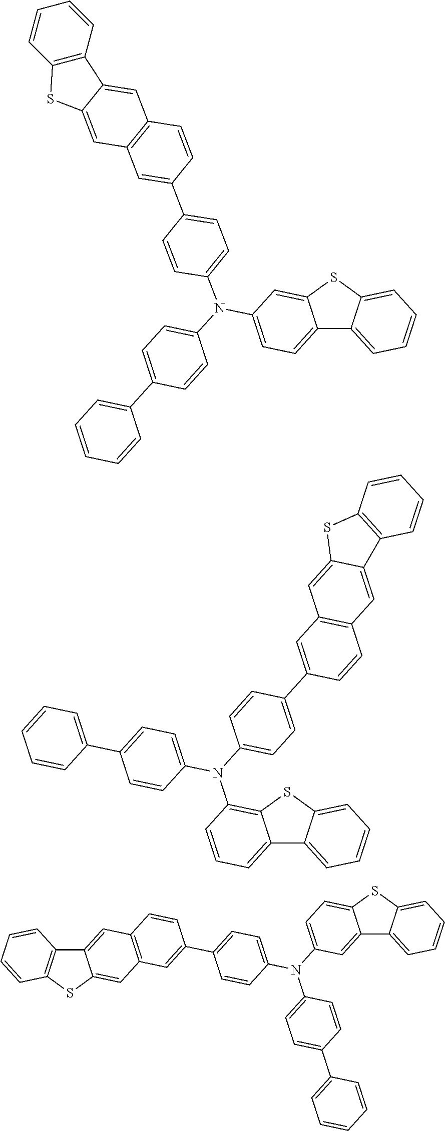 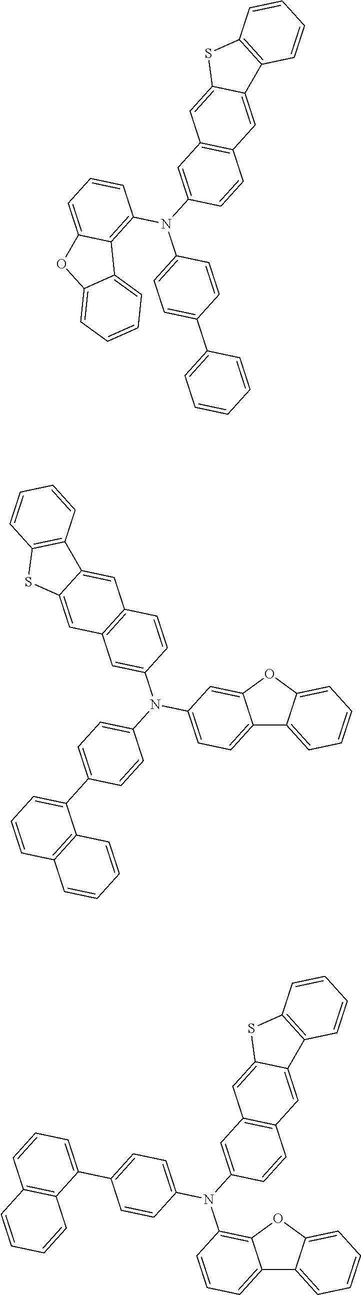 [SMILES: c1ccc(-c2ccc(N(c3ccc4cc5c(cc4c3)sc3ccccc35)c3cccc4oc5ccccc5c34)cc2)cc1 ; c1ccc2c(-c3ccc(N(c4ccc5cc6c(cc5c4)sc4ccccc46)c4ccc5c(c4)oc4ccccc45)cc3)cccc2c1 ; c1ccc2c(-c3ccc(N(c4ccc5cc6c(cc5c4)sc4ccccc46)c4cccc5c4oc4ccccc45)cc3)cccc2c1] 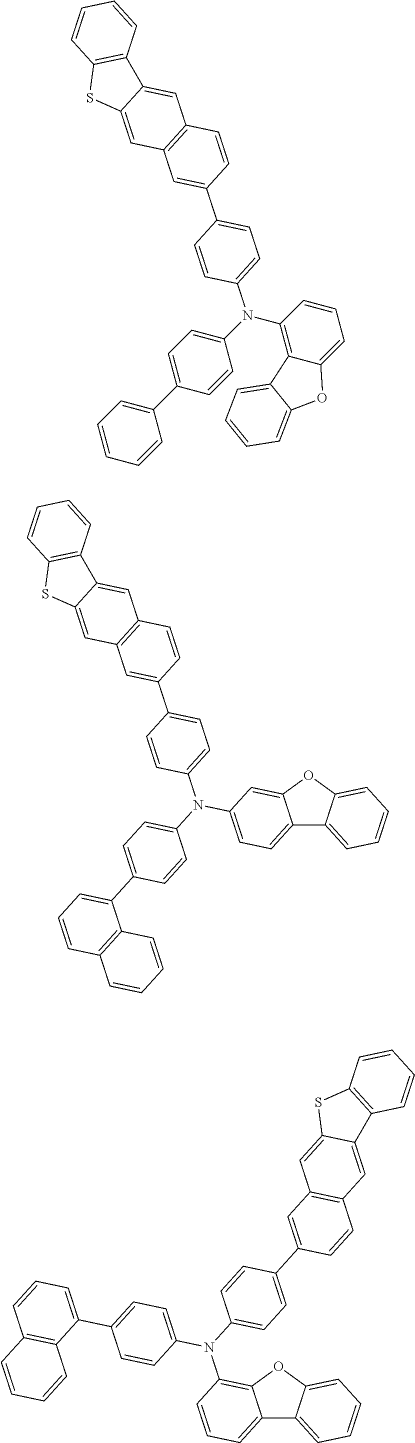 [SMILES: c1ccc(-c2ccc(N(c3ccc(-c4ccc5cc6c(cc5c4)sc4ccccc46)cc3)c3cccc4oc5ccccc5c34)cc2)cc1 ; c1ccc2c(-c3ccc(N(c4ccc(-c5ccc6cc7c(cc6c5)sc5ccccc57)cc4)c4ccc5c(c4)oc4ccccc45)cc3)cccc2c1 ; c1ccc2c(-c3ccc(N(c4ccc(-c5ccc6cc7c(cc6c5)sc5ccccc57)cc4)c4cccc5c4oc4ccccc45)cc3)cccc2c1] 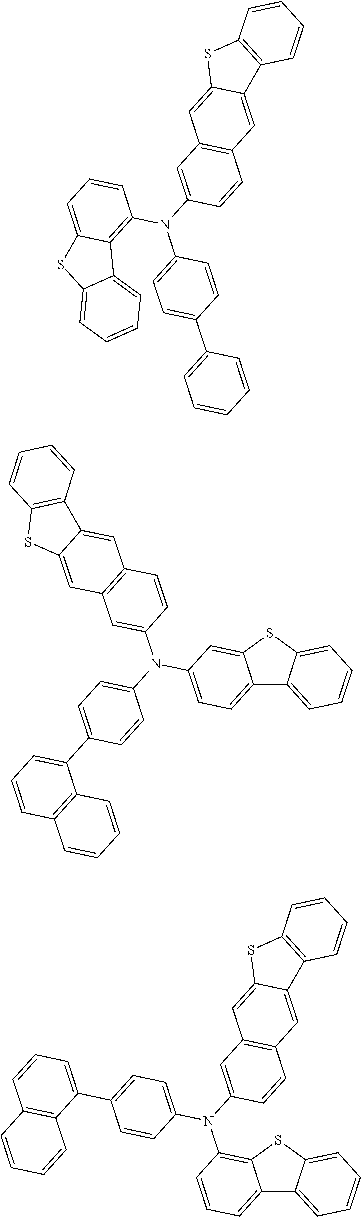 [SMILES: c1ccc(-c2ccc(N(c3ccc4cc5c(cc4c3)sc3ccccc35)c3cccc4sc5ccccc5c34)cc2)cc1 ; c1ccc2c(-c3ccc(N(c4ccc5cc6c(cc5c4)sc4ccccc46)c4ccc5c(c4)sc4ccccc45)cc3)cccc2c1 ; c1ccc2c(-c3ccc(N(c4ccc5cc6c(cc5c4)sc4ccccc46)c4cccc5c4sc4ccccc45)cc3)cccc2c1] 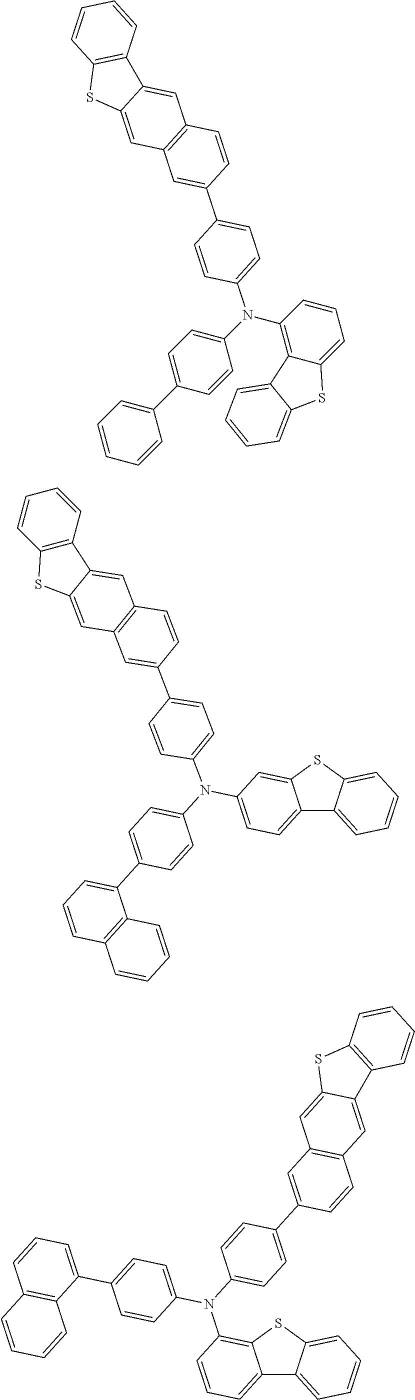 [SMILES: c1ccc(-c2ccc(N(c3ccc(-c4ccc5cc6c(cc5c4)sc4ccccc46)cc3)c3cccc4sc5ccccc5c34)cc2)cc1 ; c1ccc2c(-c3ccc(N(c4ccc(-c5ccc6cc7c(cc6c5)sc5ccccc57)cc4)c4ccc5c(c4)sc4ccccc45)cc3)cccc2c1 ; c1ccc2c(-c3ccc(N(c4ccc(-c5ccc6cc7c(cc6c5)sc5ccccc57)cc4)c4cccc5c4sc4ccccc45)cc3)cccc2c1] 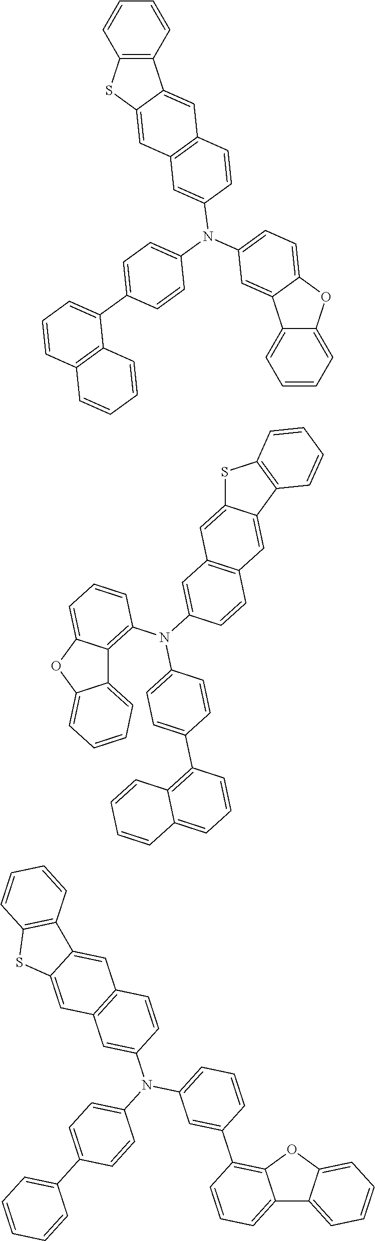 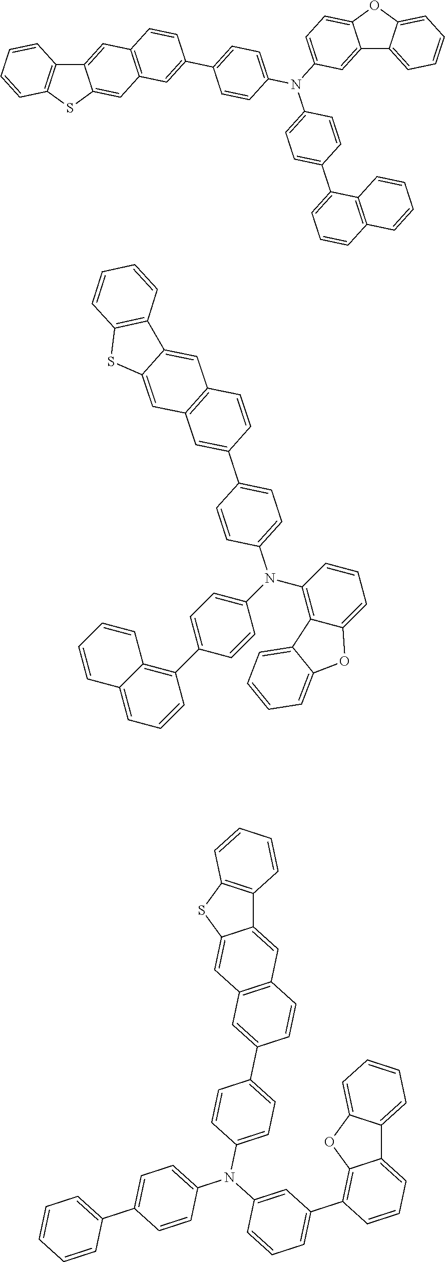 [SMILES: c1ccc2c(-c3ccc(N(c4ccc(-c5ccc6cc7c(cc6c5)sc5ccccc57)cc4)c4ccc5oc6ccccc6c5c4)cc3)cccc2c1 ; c1ccc2c(-c3ccc(N(c4ccc(-c5ccc6cc7c(cc6c5)sc5ccccc57)cc4)c4cccc5oc6ccccc6c45)cc3)cccc2c1 ; c1ccc(-c2ccc(N(c3ccc(-c4ccc5cc6c(cc5c4)sc4ccccc46)cc3)c3cccc(-c4cccc5c4oc4ccccc45)c3)cc2)cc1] 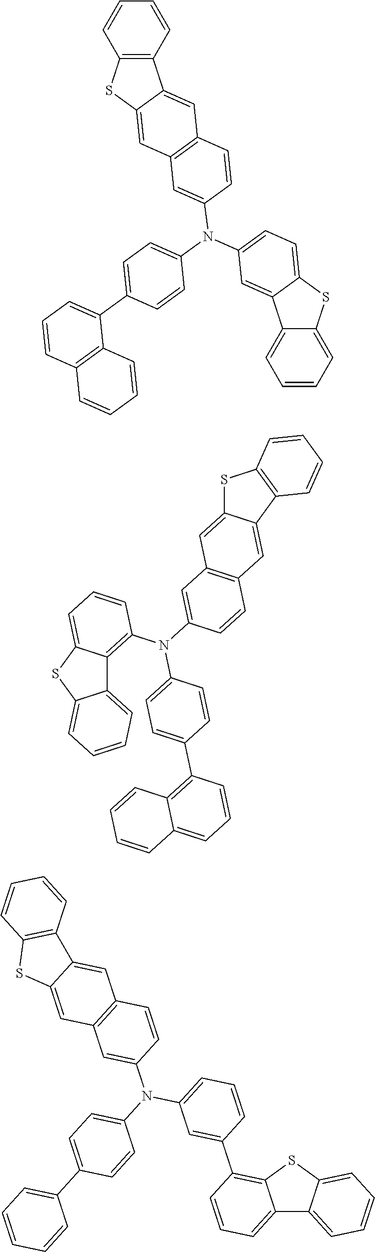 [SMILES: c1ccc2c(-c3ccc(N(c4ccc5cc6c(cc5c4)sc4ccccc46)c4ccc5sc6ccccc6c5c4)cc3)cccc2c1 ; c1ccc2c(-c3ccc(N(c4ccc5cc6c(cc5c4)sc4ccccc46)c4cccc5sc6ccccc6c45)cc3)cccc2c1 ; c1ccc(-c2ccc(N(c3cccc(-c4cccc5c4sc4ccccc45)c3)c3ccc4cc5c(cc4c3)sc3ccccc35)cc2)cc1] 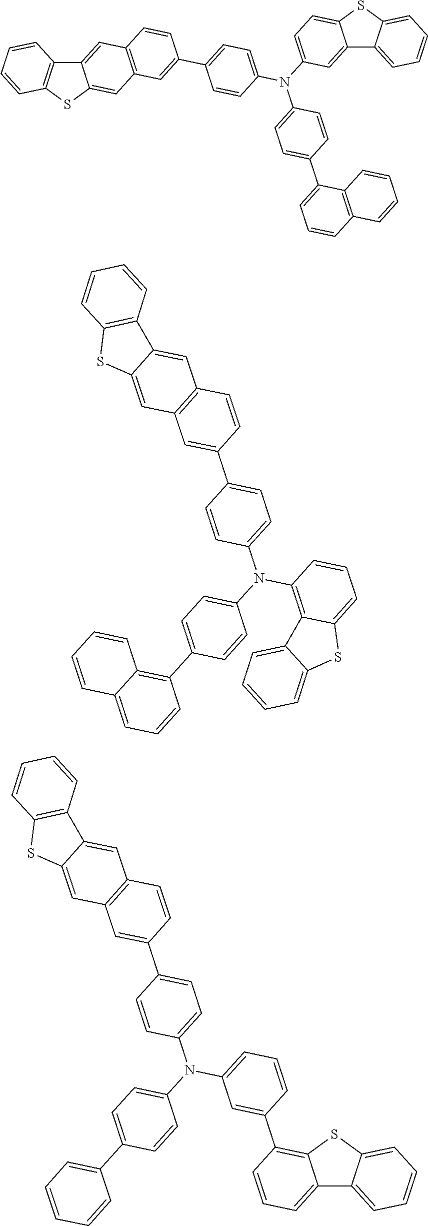 [SMILES: c1ccc2c(-c3ccc(N(c4ccc(-c5ccc6cc7c(cc6c5)sc5ccccc57)cc4)c4ccc5sc6ccccc6c5c4)cc3)cccc2c1 ; c1ccc2c(-c3ccc(N(c4ccc(-c5ccc6cc7c(cc6c5)sc5ccccc57)cc4)c4cccc5sc6ccccc6c45)cc3)cccc2c1 ; c1ccc(-c2ccc(N(c3ccc(-c4ccc5cc6c(cc5c4)sc4ccccc46)cc3)c3cccc(-c4cccc5c4sc4ccccc45)c3)cc2)cc1] 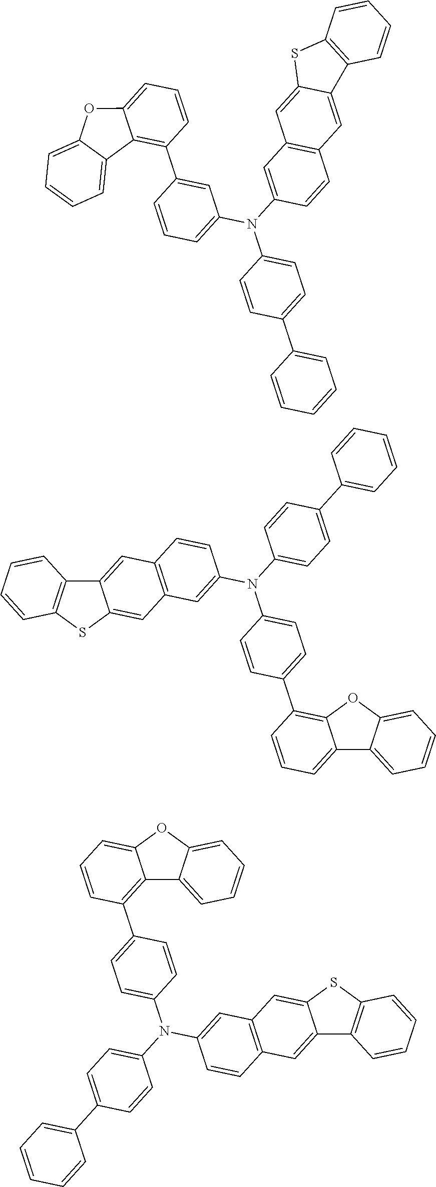 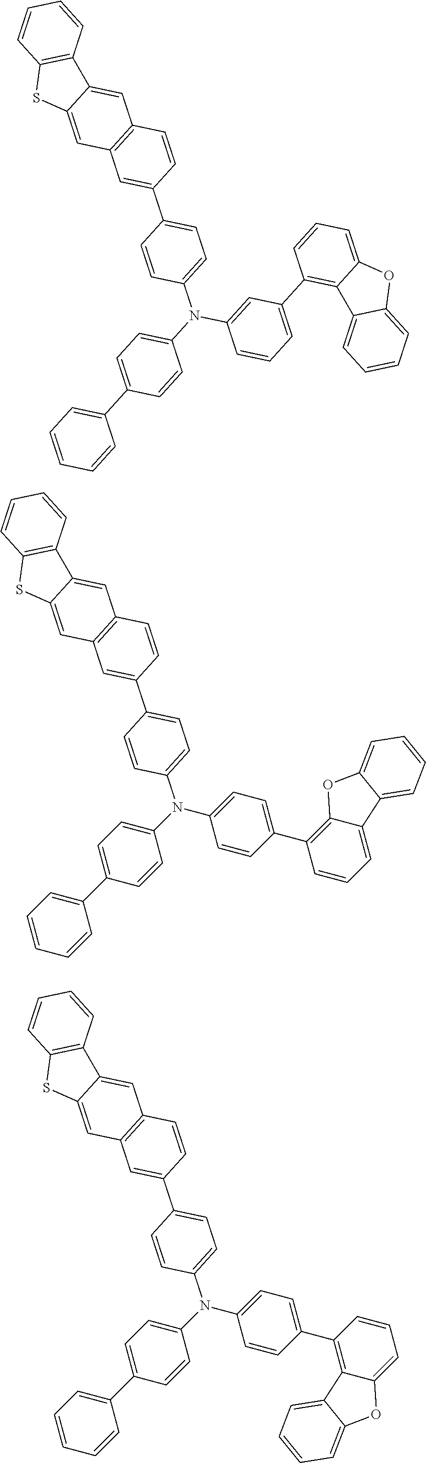 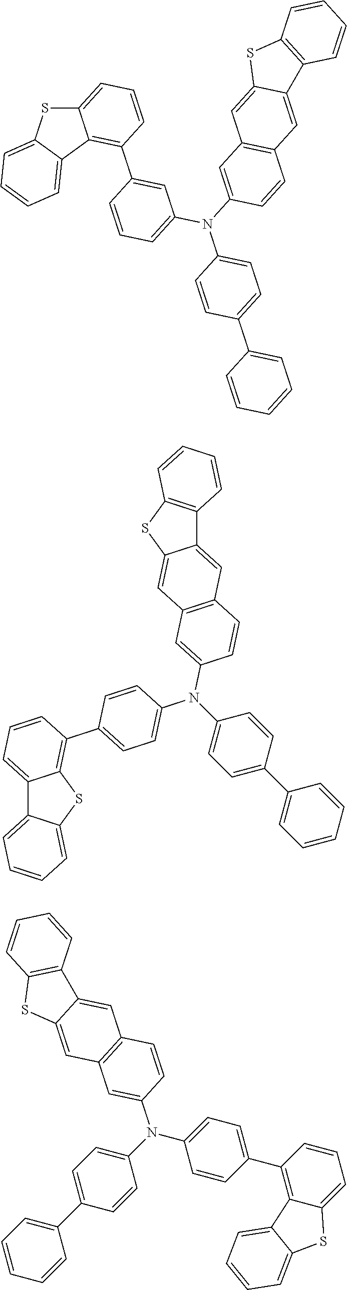 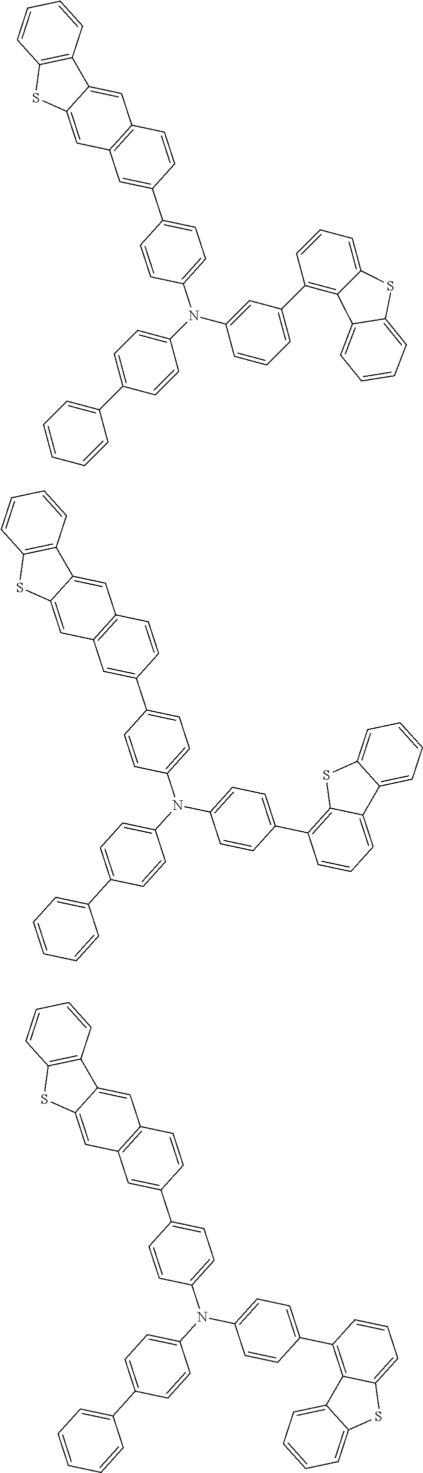 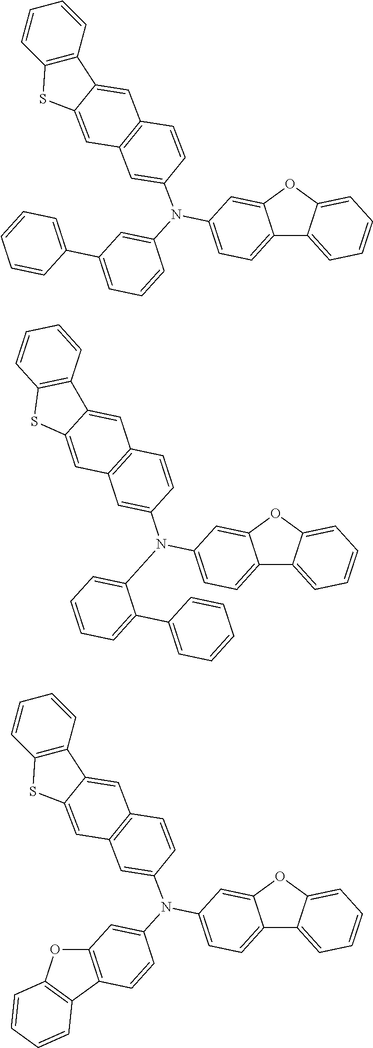 [SMILES: c1ccc(-c2cccc(N(c3ccc4cc5c(cc4c3)sc3ccccc35)c3ccc4c(c3)oc3ccccc34)c2)cc1 ; c1ccc(-c2ccccc2N(c2ccc3cc4c(cc3c2)sc2ccccc24)c2ccc3c(c2)oc2ccccc23)cc1 ; c1ccc2c(c1)oc1cc(N(c3ccc4cc5c(cc4c3)sc3ccccc35)c3ccc4c(c3)oc3ccccc34)ccc12] 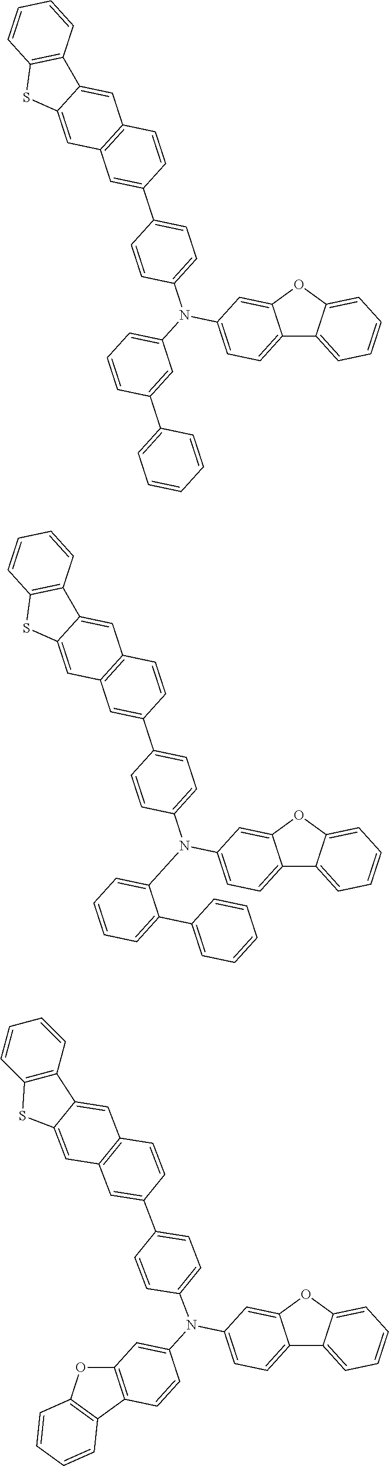 [SMILES: c1ccc(-c2cccc(N(c3ccc(-c4ccc5cc6c(cc5c4)sc4ccccc46)cc3)c3ccc4c(c3)oc3ccccc34)c2)cc1 ; c1ccc(-c2ccccc2N(c2ccc(-c3ccc4cc5c(cc4c3)sc3ccccc35)cc2)c2ccc3c(c2)oc2ccccc23)cc1 ; c1ccc2c(c1)oc1cc(N(c3ccc(-c4ccc5cc6c(cc5c4)sc4ccccc46)cc3)c3ccc4c(c3)oc3ccccc34)ccc12] 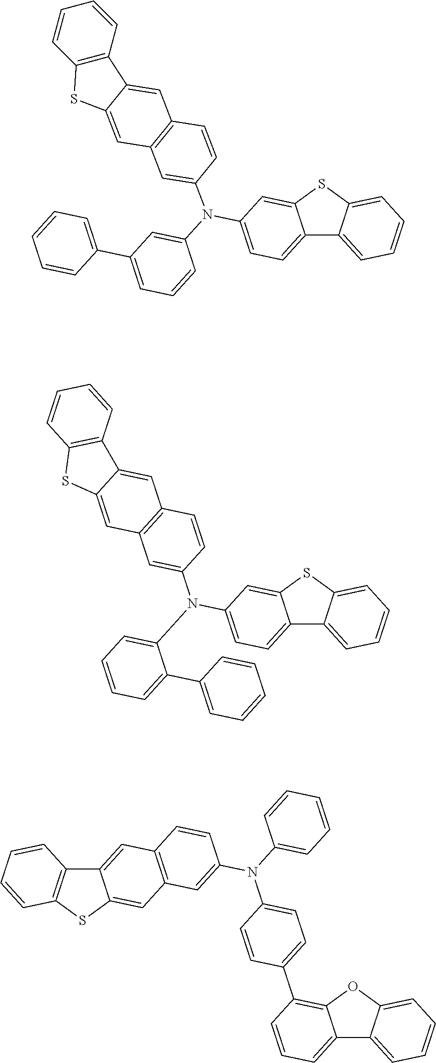 [SMILES: c1ccc(-c2cccc(N(c3ccc4cc5c(cc4c3)sc3ccccc35)c3ccc4c(c3)sc3ccccc34)c2)cc1 ; c1ccc(-c2ccccc2N(c2ccc3cc4c(cc3c2)sc2ccccc24)c2ccc3c(c2)sc2ccccc23)cc1 ; c1ccc(N(c2ccc(-c3cccc4c3oc3ccccc34)cc2)c2ccc3cc4c(cc3c2)sc2ccccc24)cc1] 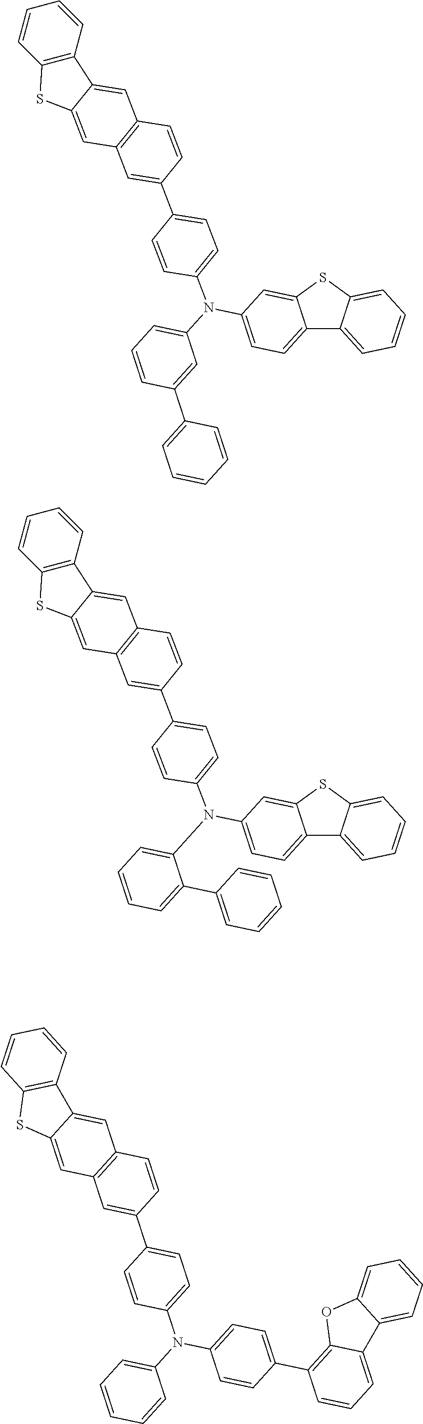 [SMILES: c1ccc(-c2cccc(N(c3ccc(-c4ccc5cc6c(cc5c4)sc4ccccc46)cc3)c3ccc4c(c3)sc3ccccc34)c2)cc1 ; c1ccc(-c2ccccc2N(c2ccc(-c3ccc4cc5c(cc4c3)sc3ccccc35)cc2)c2ccc3c(c2)sc2ccccc23)cc1 ; c1ccc(N(c2ccc(-c3ccc4cc5c(cc4c3)sc3ccccc35)cc2)c2ccc(-c3cccc4c3oc3ccccc34)cc2)cc1] 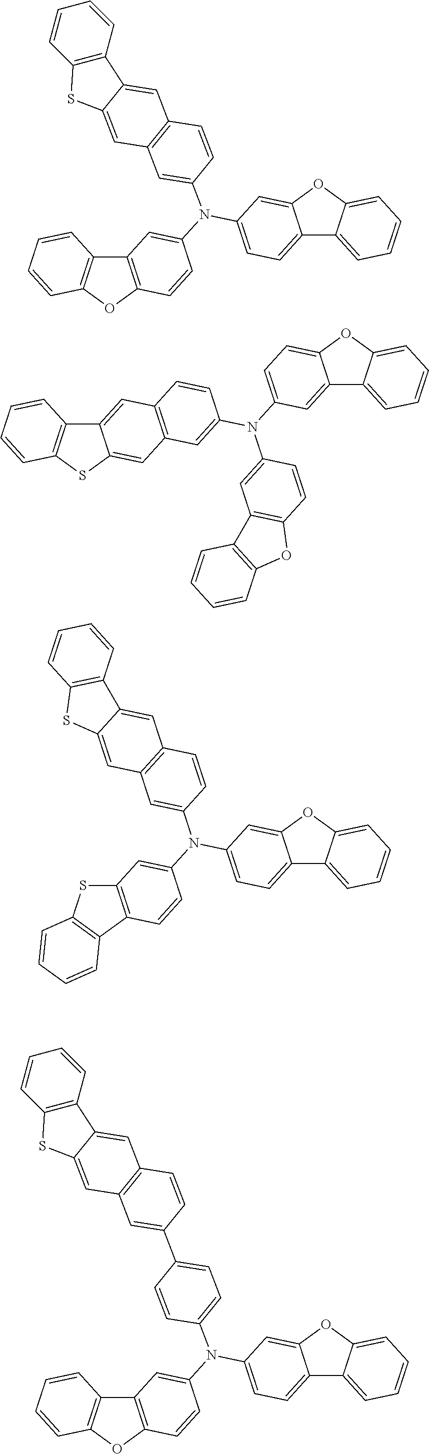 [SMILES: c1ccc2c(c1)oc1cc(N(c3ccc4cc5c(cc4c3)sc3ccccc35)c3ccc4oc5ccccc5c4c3)ccc12 ; c1ccc2c(c1)oc1ccc(N(c3ccc4cc5c(cc4c3)sc3ccccc35)c3ccc4oc5ccccc5c4c3)cc12 ; c1ccc2c(c1)oc1cc(N(c3ccc4cc5c(cc4c3)sc3ccccc35)c3ccc4c(c3)sc3ccccc34)ccc12 ; c1ccc2c(c1)oc1cc(N(c3ccc(-c4ccc5cc6c(cc5c4)sc4ccccc46)cc3)c3ccc4oc5ccccc5c4c3)ccc12] 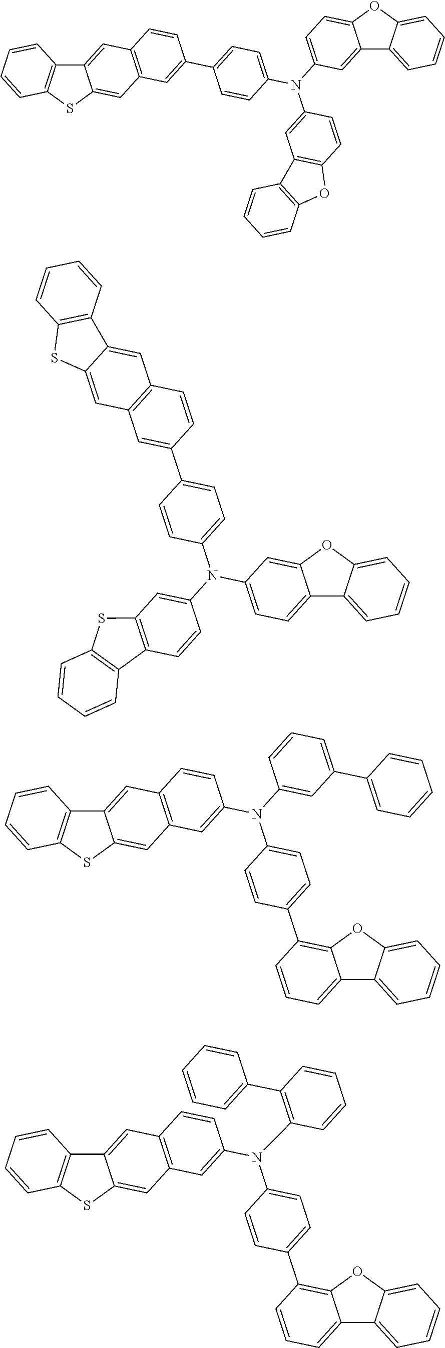 [SMILES: c1ccc2c(c1)oc1ccc(N(c3ccc(-c4ccc5cc6c(cc5c4)sc4ccccc46)cc3)c3ccc4oc5ccccc5c4c3)cc12 ; c1ccc2c(c1)oc1cc(N(c3ccc(-c4ccc5cc6c(cc5c4)sc4ccccc46)cc3)c3ccc4c(c3)sc3ccccc34)ccc12 ; c1ccc(-c2cccc(N(c3ccc(-c4cccc5c4oc4ccccc45)cc3)c3ccc4cc5c(cc4c3)sc3ccccc35)c2)cc1 ; c1ccc(-c2ccccc2N(c2ccc(-c3cccc4c3oc3ccccc34)cc2)c2ccc3cc4c(cc3c2)sc2ccccc24)cc1] 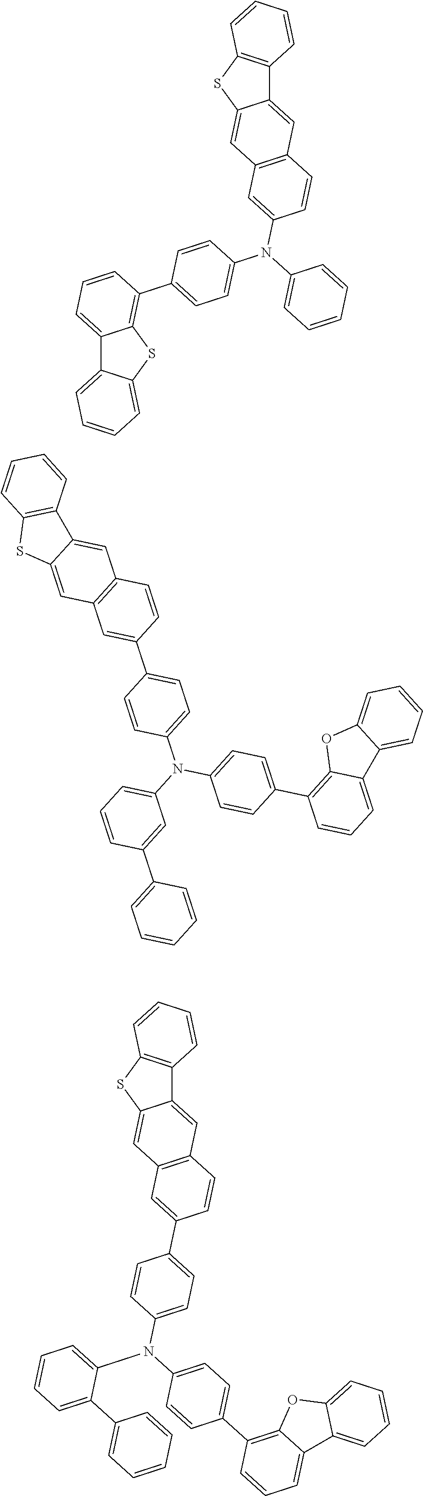 [SMILES: c1ccc(N(c2ccc(-c3cccc4c3sc3ccccc34)cc2)c2ccc3cc4c(cc3c2)sc2ccccc24)cc1 ; c1ccc(-c2cccc(N(c3ccc(-c4ccc5cc6c(cc5c4)sc4ccccc46)cc3)c3ccc(-c4cccc5c4oc4ccccc45)cc3)c2)cc1 ; c1ccc(-c2ccccc2N(c2ccc(-c3ccc4cc5c(cc4c3)sc3ccccc35)cc2)c2ccc(-c3cccc4c3oc3ccccc34)cc2)cc1] 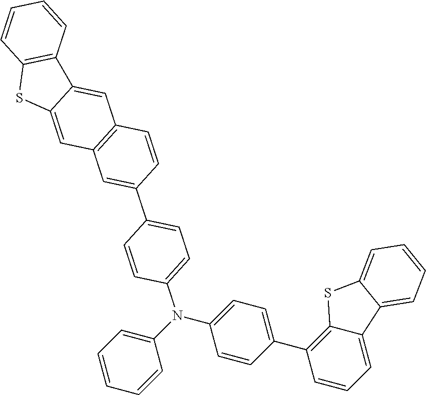 [SMILES: c1ccc(N(c2ccc(-c3ccc4cc5c(cc4c3)sc3ccccc35)cc2)c2ccc(-c3cccc4c3sc3ccccc34)cc2)cc1] 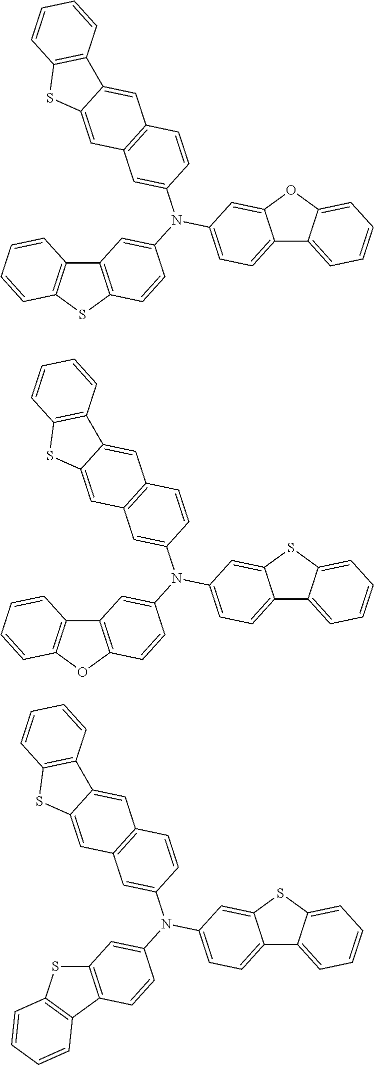 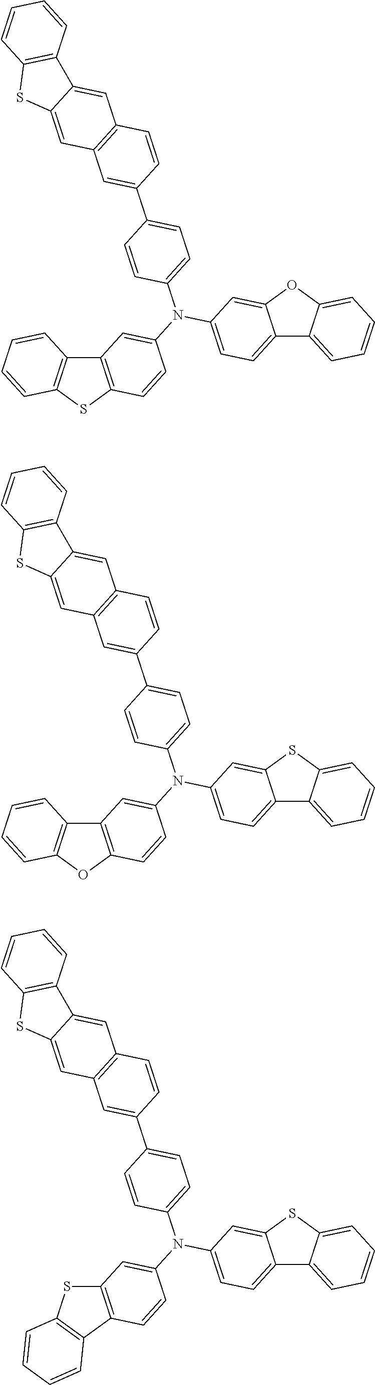 [SMILES: c1ccc2c(c1)oc1cc(N(c3ccc(-c4ccc5cc6c(cc5c4)sc4ccccc46)cc3)c3ccc4sc5ccccc5c4c3)ccc12 ; c1ccc2c(c1)oc1ccc(N(c3ccc(-c4ccc5cc6c(cc5c4)sc4ccccc46)cc3)c3ccc4c(c3)sc3ccccc34)cc12 ; c1ccc2c(c1)sc1cc(N(c3ccc(-c4ccc5cc6c(cc5c4)sc4ccccc46)cc3)c3ccc4c(c3)sc3ccccc34)ccc12] 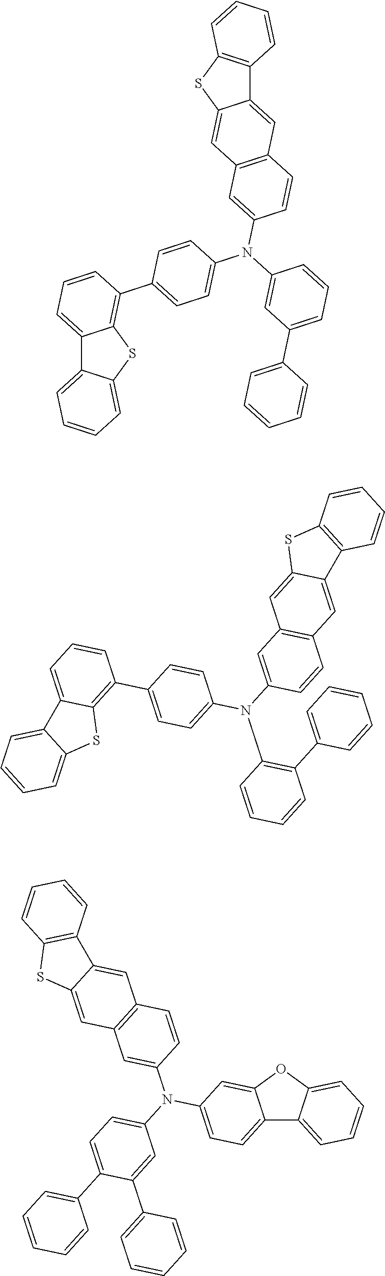 [SMILES: c1ccc(-c2cccc(N(c3ccc(-c4cccc5c4sc4ccccc45)cc3)c3ccc4cc5c(cc4c3)sc3ccccc35)c2)cc1 ; c1ccc(-c2ccccc2N(c2ccc(-c3cccc4c3sc3ccccc34)cc2)c2ccc3cc4c(cc3c2)sc2ccccc24)cc1 ; c1ccc(-c2ccc(N(c3ccc4cc5c(cc4c3)sc3ccccc35)c3ccc4c(c3)oc3ccccc34)cc2-c2ccccc2)cc1] 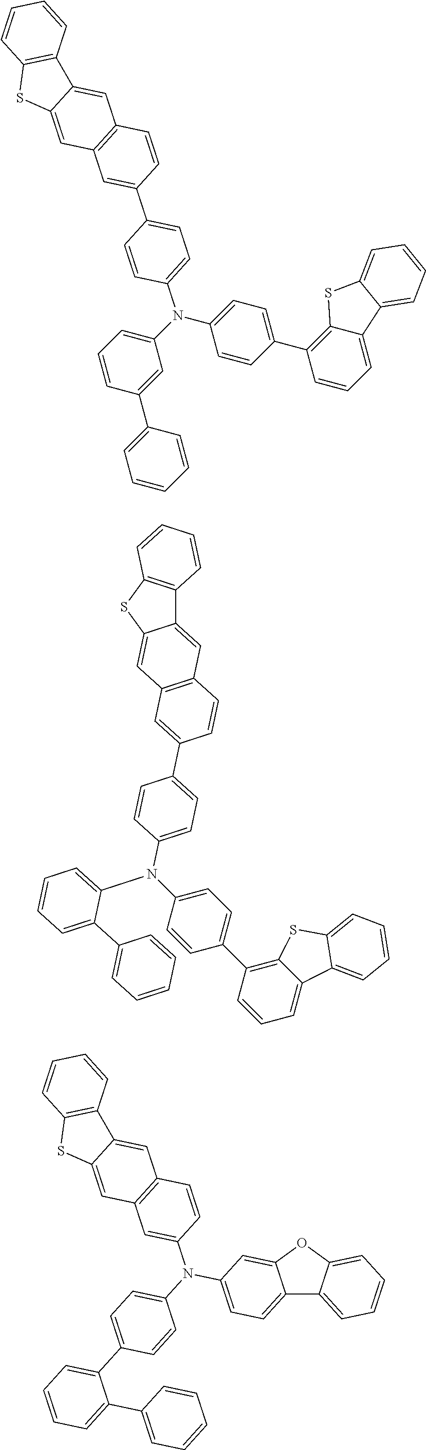 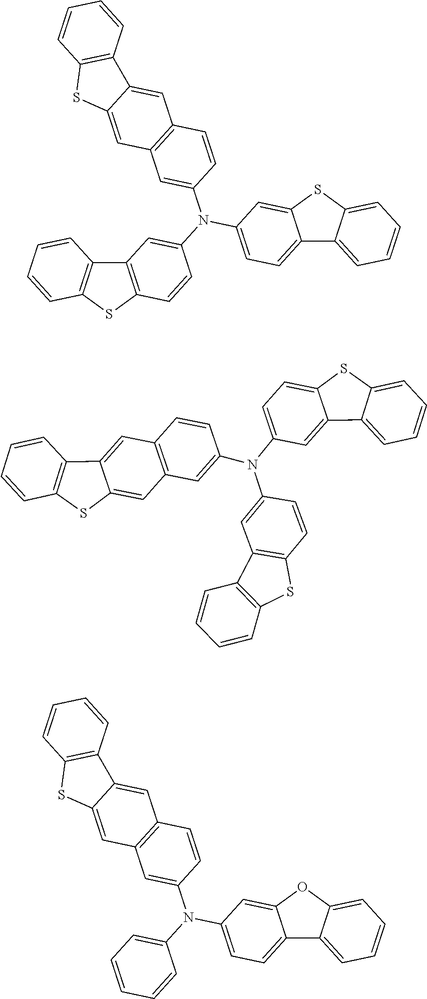 [SMILES: c1ccc2c(c1)sc1cc(N(c3ccc4cc5c(cc4c3)sc3ccccc35)c3ccc4sc5ccccc5c4c3)ccc12 ; c1ccc2c(c1)sc1ccc(N(c3ccc4cc5c(cc4c3)sc3ccccc35)c3ccc4sc5ccccc5c4c3)cc12 ; c1ccc(N(c2ccc3cc4c(cc3c2)sc2ccccc24)c2ccc3c(c2)oc2ccccc23)cc1] 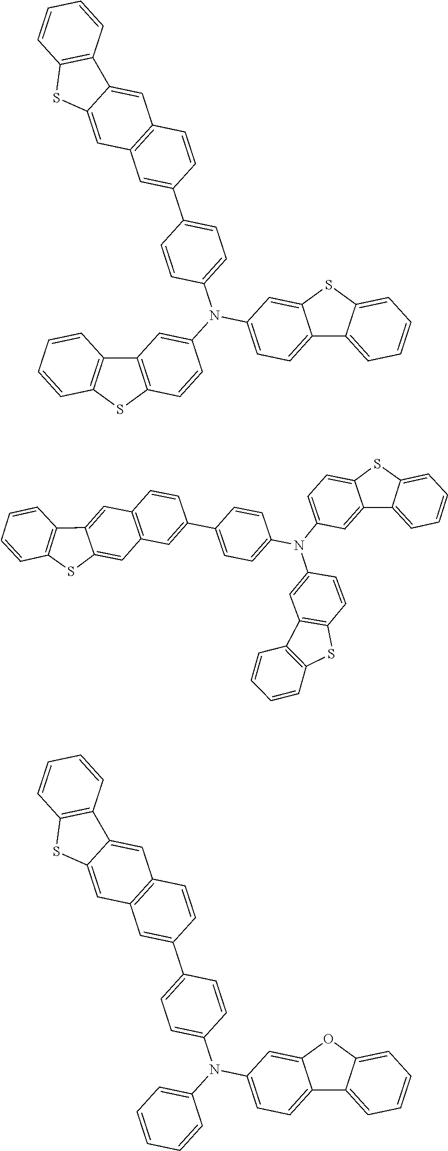 [SMILES: c1ccc2c(c1)sc1cc(N(c3ccc(-c4ccc5cc6c(cc5c4)sc4ccccc46)cc3)c3ccc4sc5ccccc5c4c3)ccc12 ; c1ccc2c(c1)sc1ccc(N(c3ccc(-c4ccc5cc6c(cc5c4)sc4ccccc46)cc3)c3ccc4sc5ccccc5c4c3)cc12 ; c1ccc(N(c2ccc(-c3ccc4cc5c(cc4c3)sc3ccccc35)cc2)c2ccc3c(c2)oc2ccccc23)cc1] 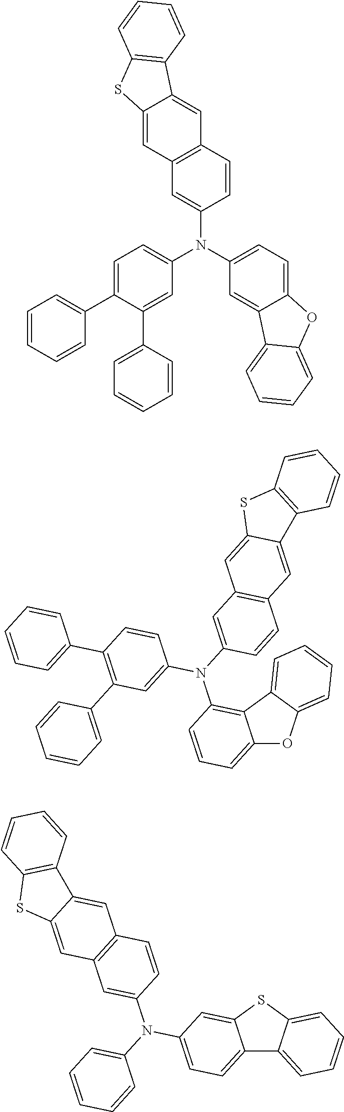 [SMILES: c1ccc(-c2ccc(N(c3ccc4cc5c(cc4c3)sc3ccccc35)c3ccc4oc5ccccc5c4c3)cc2-c2ccccc2)cc1 ; c1ccc(-c2ccc(N(c3ccc4cc5c(cc4c3)sc3ccccc35)c3cccc4oc5ccccc5c34)cc2-c2ccccc2)cc1 ; c1ccc(N(c2ccc3cc4c(cc3c2)sc2ccccc24)c2ccc3c(c2)sc2ccccc23)cc1] 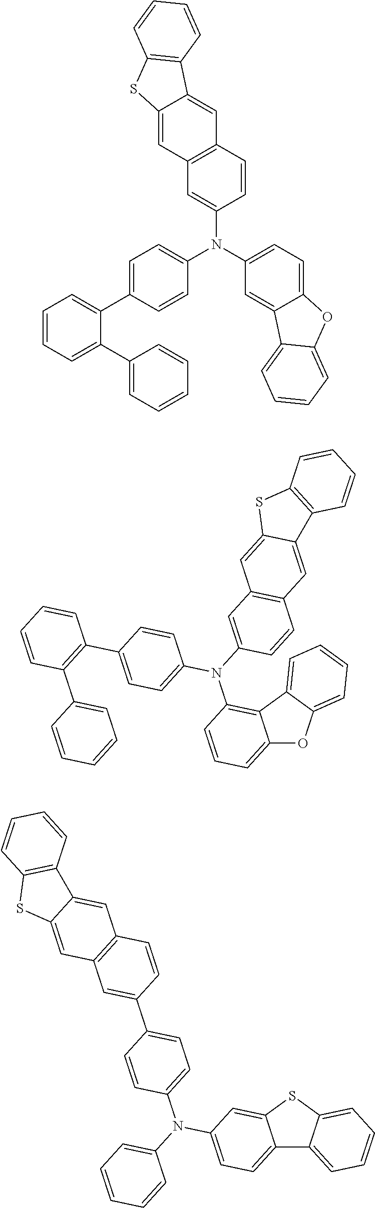 [SMILES: c1ccc(-c2ccccc2-c2ccc(N(c3ccc4cc5c(cc4c3)sc3ccccc35)c3ccc4oc5ccccc5c4c3)cc2)cc1 ; c1ccc(-c2ccccc2-c2ccc(N(c3ccc4cc5c(cc4c3)sc3ccccc35)c3cccc4oc5ccccc5c34)cc2)cc1 ; c1ccc(N(c2ccc(-c3ccc4cc5c(cc4c3)sc3ccccc35)cc2)c2ccc3c(c2)sc2ccccc23)cc1] 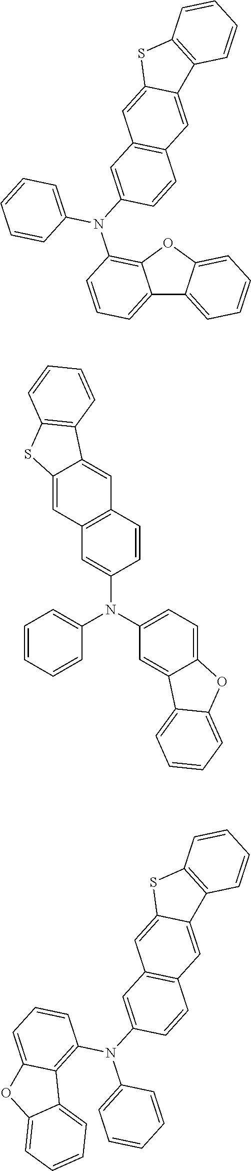 [SMILES: c1ccc(N(c2ccc3cc4c(cc3c2)sc2ccccc24)c2cccc3c2oc2ccccc23)cc1 ; c1ccc(N(c2ccc3cc4c(cc3c2)sc2ccccc24)c2ccc3oc4ccccc4c3c2)cc1 ; c1ccc(N(c2ccc3cc4c(cc3c2)sc2ccccc24)c2cccc3oc4ccccc4c23)cc1] 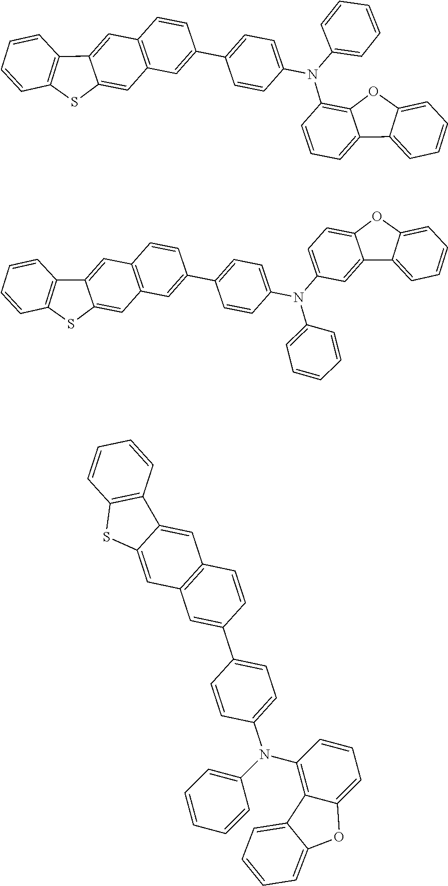 [SMILES: c1ccc(N(c2ccc(-c3ccc4cc5c(cc4c3)sc3ccccc35)cc2)c2cccc3c2oc2ccccc23)cc1 ; c1ccc(N(c2ccc(-c3ccc4cc5c(cc4c3)sc3ccccc35)cc2)c2ccc3oc4ccccc4c3c2)cc1 ; c1ccc(N(c2ccc(-c3ccc4cc5c(cc4c3)sc3ccccc35)cc2)c2cccc3oc4ccccc4c23)cc1] 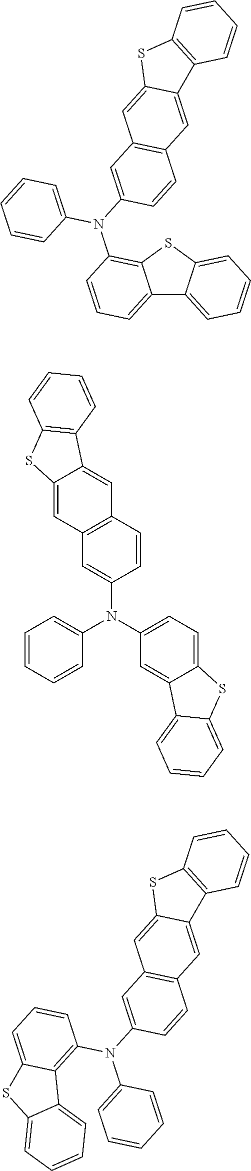 [SMILES: c1ccc(N(c2ccc3cc4c(cc3c2)sc2ccccc24)c2cccc3c2sc2ccccc23)cc1 ; c1ccc(N(c2ccc3cc4c(cc3c2)sc2ccccc24)c2ccc3sc4ccccc4c3c2)cc1 ; c1ccc(N(c2ccc3cc4c(cc3c2)sc2ccccc24)c2cccc3sc4ccccc4c23)cc1] 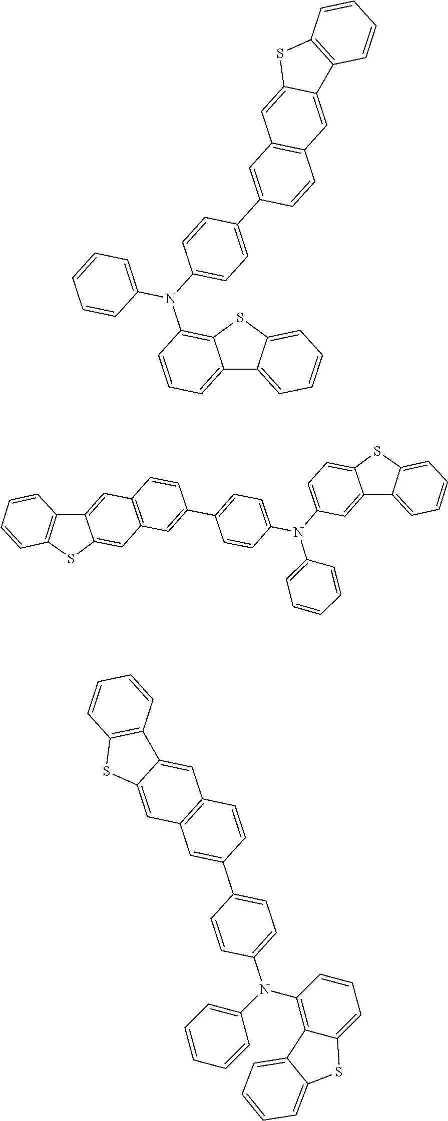 [SMILES: c1ccc(N(c2ccc(-c3ccc4cc5c(cc4c3)sc3ccccc35)cc2)c2cccc3c2sc2ccccc23)cc1 ; c1ccc(N(c2ccc(-c3ccc4cc5c(cc4c3)sc3ccccc35)cc2)c2ccc3sc4ccccc4c3c2)cc1 ; c1ccc(N(c2ccc(-c3ccc4cc5c(cc4c3)sc3ccccc35)cc2)c2cccc3sc4ccccc4c23)cc1] 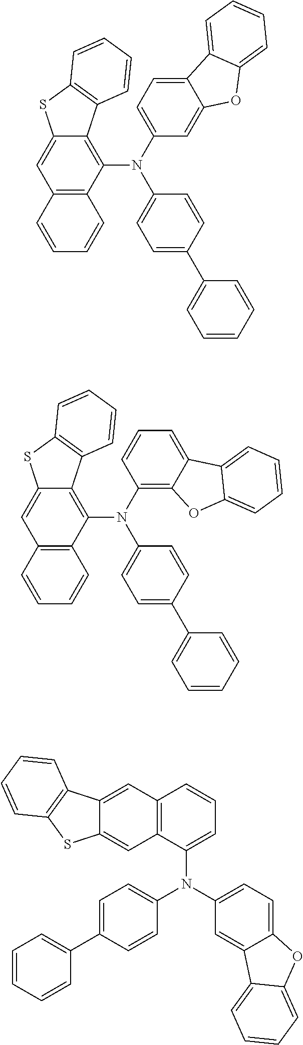 [SMILES: c1ccc(-c2ccc(N(c3ccc4c(c3)oc3ccccc34)c3c4ccccc4cc4sc5ccccc5c34)cc2)cc1 ; c1ccc(-c2ccc(N(c3cccc4c3oc3ccccc34)c3c4ccccc4cc4sc5ccccc5c34)cc2)cc1 ; c1ccc(-c2ccc(N(c3ccc4oc5ccccc5c4c3)c3cccc4cc5c(cc34)sc3ccccc35)cc2)cc1] 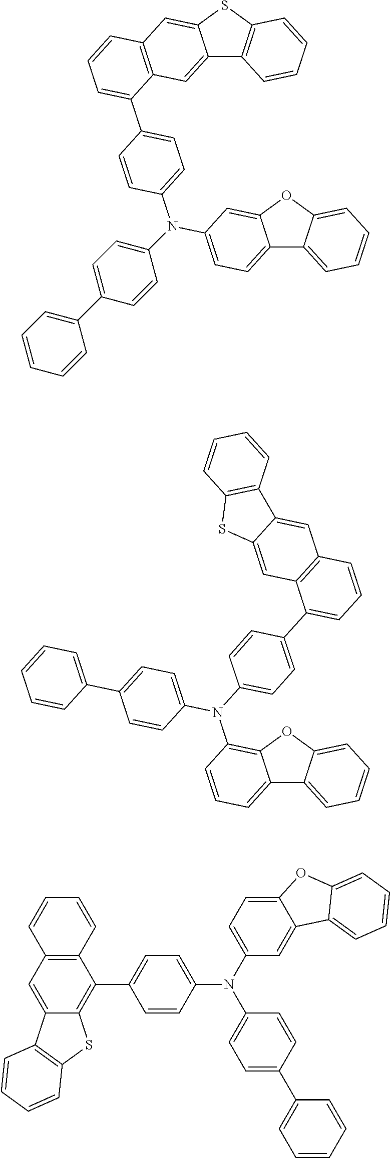 [SMILES: c1ccc(-c2ccc(N(c3ccc(-c4cccc5cc6sc7ccccc7c6cc45)cc3)c3ccc4c(c3)oc3ccccc34)cc2)cc1 ; c1ccc(-c2ccc(N(c3ccc(-c4cccc5cc6c(cc45)sc4ccccc46)cc3)c3cccc4c3oc3ccccc34)cc2)cc1 ; c1ccc(-c2ccc(N(c3ccc(-c4c5ccccc5cc5c4sc4ccccc45)cc3)c3ccc4oc5ccccc5c4c3)cc2)cc1] 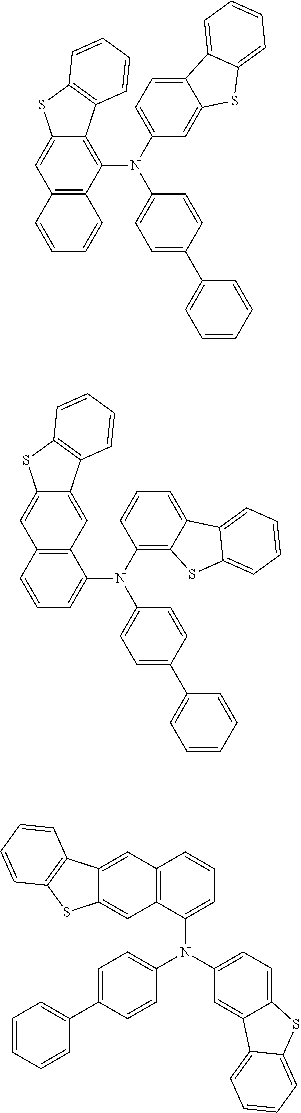 [SMILES: c1ccc(-c2ccc(N(c3ccc4c(c3)sc3ccccc34)c3c4ccccc4cc4sc5ccccc5c34)cc2)cc1 ; c1ccc(-c2ccc(N(c3cccc4cc5sc6ccccc6c5cc34)c3cccc4c3sc3ccccc34)cc2)cc1 ; c1ccc(-c2ccc(N(c3ccc4sc5ccccc5c4c3)c3cccc4cc5c(cc34)sc3ccccc35)cc2)cc1] 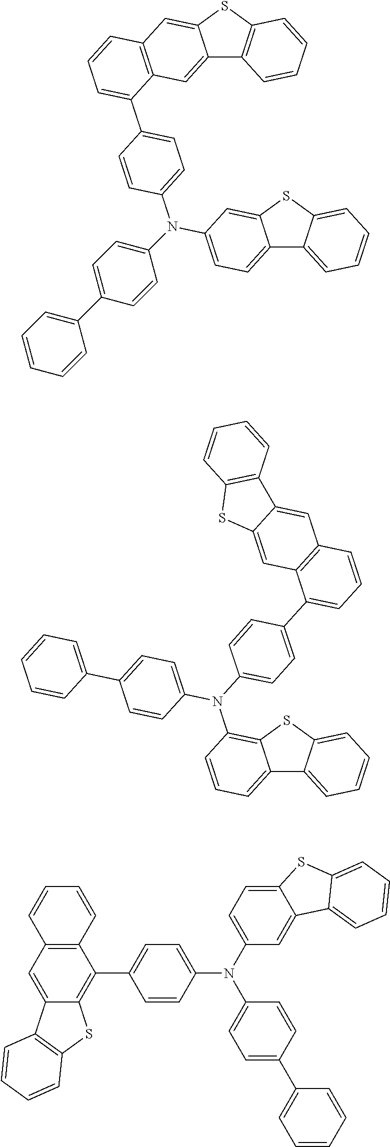 [SMILES: c1ccc(-c2ccc(N(c3ccc(-c4cccc5cc6sc7ccccc7c6cc45)cc3)c3ccc4c(c3)sc3ccccc34)cc2)cc1 ; c1ccc(-c2ccc(N(c3ccc(-c4cccc5cc6c(cc45)sc4ccccc46)cc3)c3cccc4c3sc3ccccc34)cc2)cc1 ; c1ccc(-c2ccc(N(c3ccc(-c4c5ccccc5cc5c4sc4ccccc45)cc3)c3ccc4sc5ccccc5c4c3)cc2)cc1] 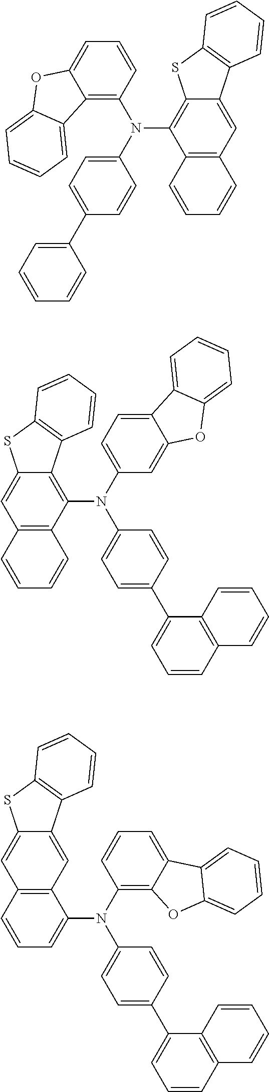 [SMILES: c1ccc(-c2ccc(N(c3c4ccccc4cc4c3sc3ccccc34)c3cccc4oc5ccccc5c34)cc2)cc1 ; c1ccc2c(-c3ccc(N(c4ccc5c(c4)oc4ccccc45)c4c5ccccc5cc5sc6ccccc6c45)cc3)cccc2c1 ; c1ccc2c(-c3ccc(N(c4cccc5cc6sc7ccccc7c6cc45)c4cccc5c4oc4ccccc45)cc3)cccc2c1] 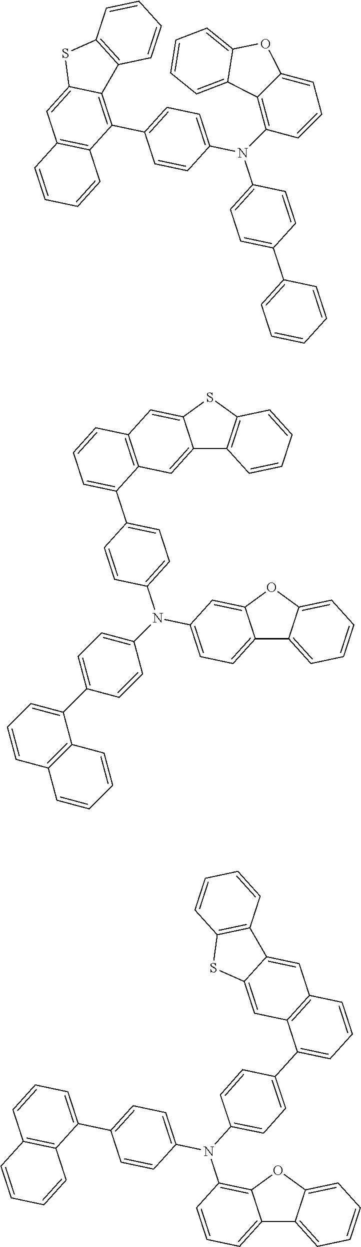 [SMILES: c1ccc(-c2ccc(N(c3ccc(-c4c5ccccc5cc5sc6ccccc6c45)cc3)c3cccc4oc5ccccc5c34)cc2)cc1 ; c1ccc2c(-c3ccc(N(c4ccc(-c5cccc6cc7sc8ccccc8c7cc56)cc4)c4ccc5c(c4)oc4ccccc45)cc3)cccc2c1 ; c1ccc2c(-c3ccc(N(c4ccc(-c5cccc6cc7c(cc56)sc5ccccc57)cc4)c4cccc5c4oc4ccccc45)cc3)cccc2c1] 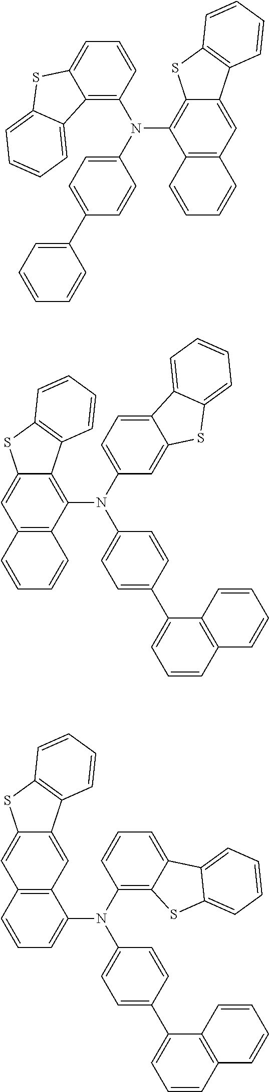 [SMILES: c1ccc(-c2ccc(N(c3c4ccccc4cc4c3sc3ccccc34)c3cccc4sc5ccccc5c34)cc2)cc1 ; c1ccc2c(-c3ccc(N(c4ccc5c(c4)sc4ccccc45)c4c5ccccc5cc5sc6ccccc6c45)cc3)cccc2c1 ; c1ccc2c(-c3ccc(N(c4cccc5cc6sc7ccccc7c6cc45)c4cccc5c4sc4ccccc45)cc3)cccc2c1] 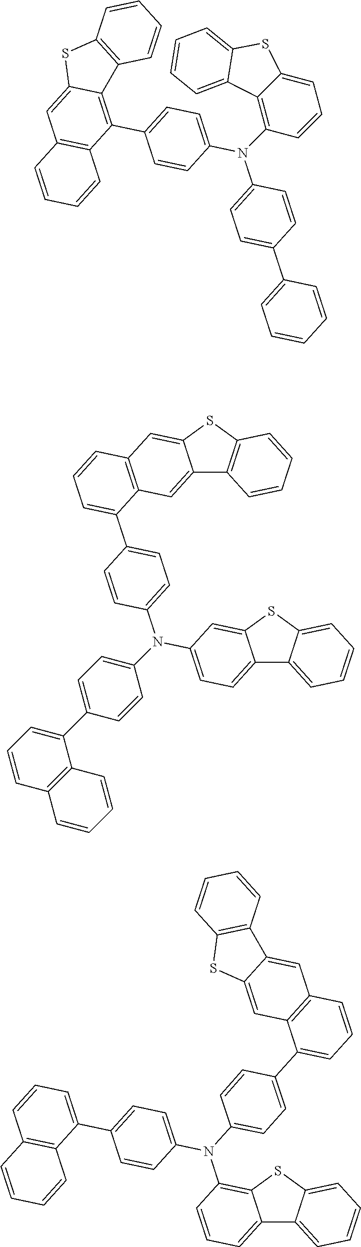 [SMILES: c1ccc(-c2ccc(N(c3ccc(-c4c5ccccc5cc5sc6ccccc6c45)cc3)c3cccc4sc5ccccc5c34)cc2)cc1 ; c1ccc2c(-c3ccc(N(c4ccc(-c5cccc6cc7sc8ccccc8c7cc56)cc4)c4ccc5c(c4)sc4ccccc45)cc3)cccc2c1 ; c1ccc2c(-c3ccc(N(c4ccc(-c5cccc6cc7c(cc56)sc5ccccc57)cc4)c4cccc5c4sc4ccccc45)cc3)cccc2c1] 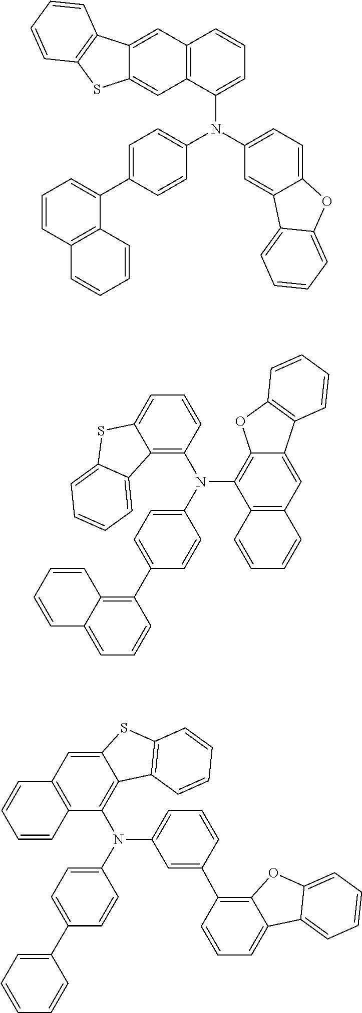 [SMILES: c1ccc2c(-c3ccc(N(c4ccc5oc6ccccc6c5c4)c4cccc5cc6c(cc45)sc4ccccc46)cc3)cccc2c1 ; c1ccc2c(-c3ccc(N(c4c5ccccc5cc5c4oc4ccccc45)c4cccc5sc6ccccc6c45)cc3)cccc2c1 ; c1ccc(-c2ccc(N(c3cccc(-c4cccc5c4oc4ccccc45)c3)c3c4ccccc4cc4sc5ccccc5c34)cc2)cc1] 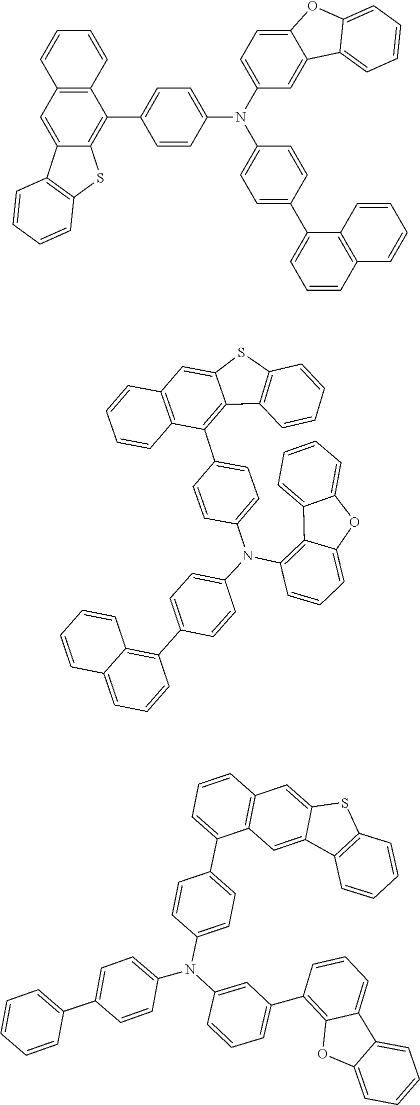 [SMILES: c1ccc2c(-c3ccc(N(c4ccc(-c5c6ccccc6cc6c5sc5ccccc56)cc4)c4ccc5oc6ccccc6c5c4)cc3)cccc2c1 ; c1ccc2c(-c3ccc(N(c4ccc(-c5c6ccccc6cc6sc7ccccc7c56)cc4)c4cccc5oc6ccccc6c45)cc3)cccc2c1 ; c1ccc(-c2ccc(N(c3ccc(-c4cccc5cc6sc7ccccc7c6cc45)cc3)c3cccc(-c4cccc5c4oc4ccccc45)c3)cc2)cc1] 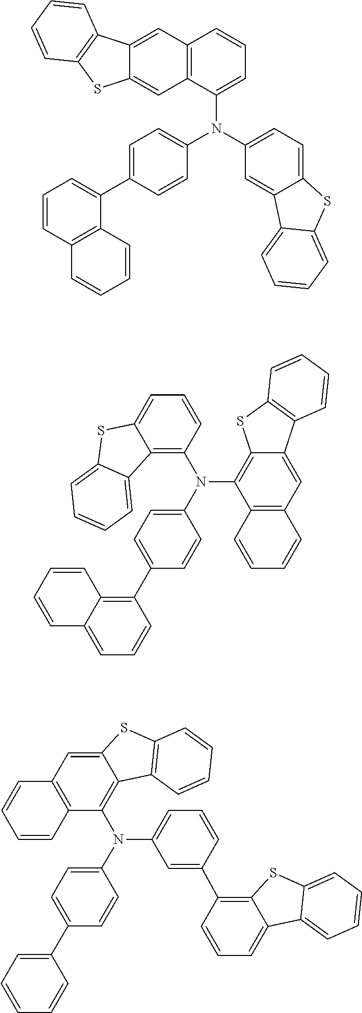 [SMILES: c1ccc2c(-c3ccc(N(c4ccc5sc6ccccc6c5c4)c4cccc5cc6c(cc45)sc4ccccc46)cc3)cccc2c1 ; c1ccc2c(-c3ccc(N(c4c5ccccc5cc5c4sc4ccccc45)c4cccc5sc6ccccc6c45)cc3)cccc2c1 ; c1ccc(-c2ccc(N(c3cccc(-c4cccc5c4sc4ccccc45)c3)c3c4ccccc4cc4sc5ccccc5c34)cc2)cc1] 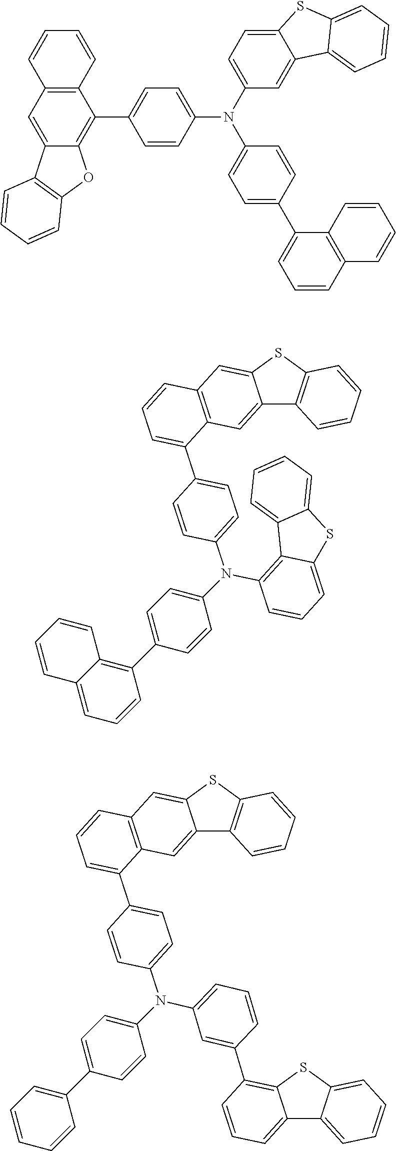 [SMILES: c1ccc2c(-c3ccc(N(c4ccc(-c5c6ccccc6cc6c5oc5ccccc56)cc4)c4ccc5sc6ccccc6c5c4)cc3)cccc2c1 ; c1ccc2c(-c3ccc(N(c4ccc(-c5cccc6cc7sc8ccccc8c7cc56)cc4)c4cccc5sc6ccccc6c45)cc3)cccc2c1 ; c1ccc(-c2ccc(N(c3ccc(-c4cccc5cc6sc7ccccc7c6cc45)cc3)c3cccc(-c4cccc5c4sc4ccccc45)c3)cc2)cc1] 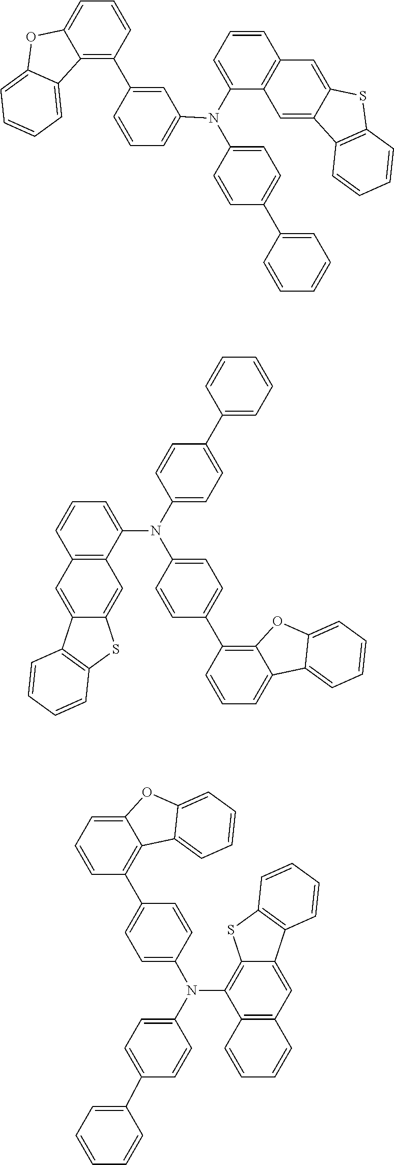 [SMILES: c1ccc(-c2ccc(N(c3cccc(-c4cccc5oc6ccccc6c45)c3)c3cccc4cc5sc6ccccc6c5cc34)cc2)cc1 ; c1ccc(-c2ccc(N(c3ccc(-c4cccc5c4oc4ccccc45)cc3)c3cccc4cc5c(cc34)sc3ccccc35)cc2)cc1 ; c1ccc(-c2ccc(N(c3ccc(-c4cccc5oc6ccccc6c45)cc3)c3c4ccccc4cc4c3sc3ccccc34)cc2)cc1] 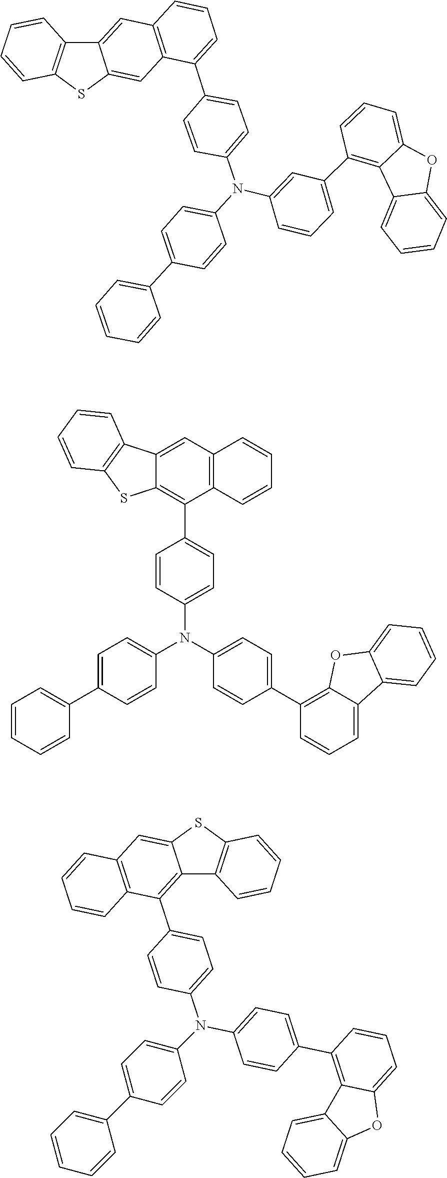 [SMILES: c1ccc(-c2ccc(N(c3ccc(-c4cccc5cc6c(cc45)sc4ccccc46)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4c5ccccc5cc5c4sc4ccccc45)cc3)cc2)cc1 ; c1ccc(-c2ccc(N(c3ccc(-c4cccc5oc6ccccc6c45)cc3)c3ccc(-c4c5ccccc5cc5sc6ccccc6c45)cc3)cc2)cc1] 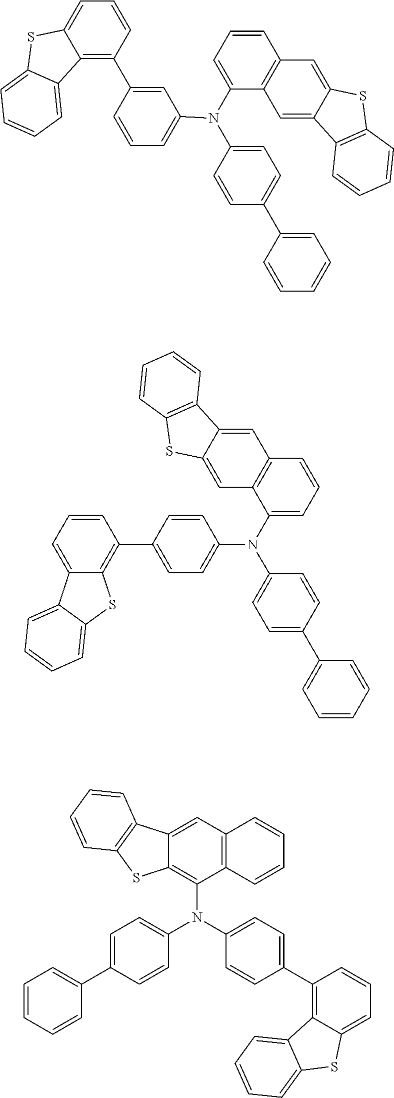 [SMILES: c1ccc(-c2ccc(N(c3cccc(-c4cccc5sc6ccccc6c45)c3)c3cccc4cc5sc6ccccc6c5cc34)cc2)cc1 ; c1ccc(-c2ccc(N(c3ccc(-c4cccc5c4sc4ccccc45)cc3)c3cccc4cc5c(cc34)sc3ccccc35)cc2)cc1 ; c1ccc(-c2ccc(N(c3ccc(-c4cccc5sc6ccccc6c45)cc3)c3c4ccccc4cc4c3sc3ccccc34)cc2)cc1] 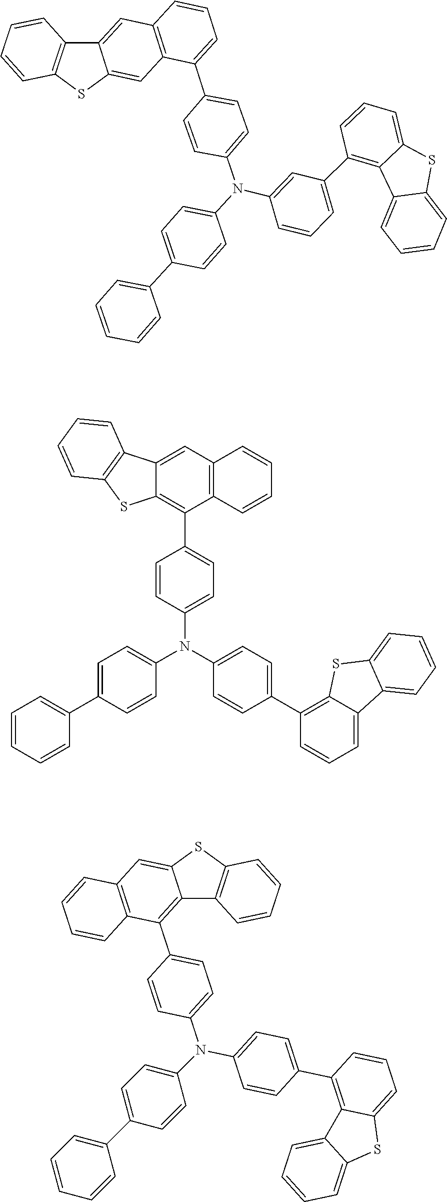 [SMILES: c1ccc(-c2ccc(N(c3ccc(-c4cccc5cc6c(cc45)sc4ccccc46)cc3)c3cccc(-c4cccc5sc6ccccc6c45)c3)cc2)cc1 ; c1ccc(-c2ccc(N(c3ccc(-c4cccc5c4sc4ccccc45)cc3)c3ccc(-c4c5ccccc5cc5c4sc4ccccc45)cc3)cc2)cc1 ; c1ccc(-c2ccc(N(c3ccc(-c4cccc5sc6ccccc6c45)cc3)c3ccc(-c4c5ccccc5cc5sc6ccccc6c45)cc3)cc2)cc1] 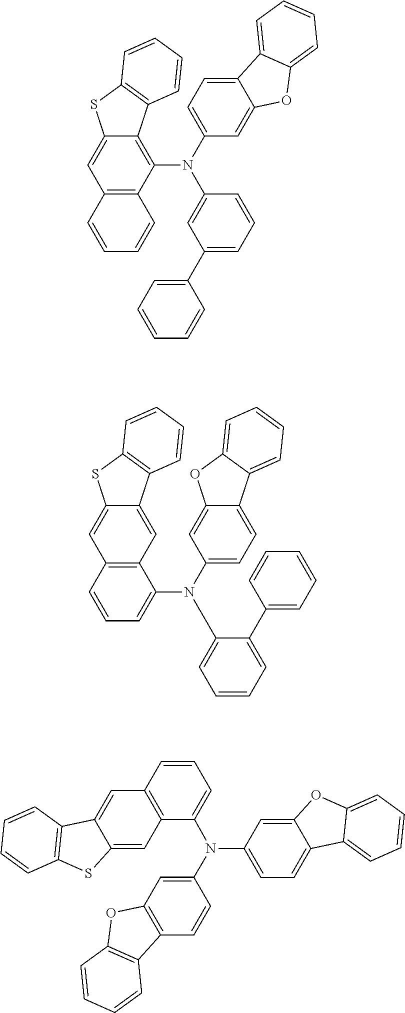 [SMILES: c1ccc(-c2cccc(N(c3ccc4c(c3)oc3ccccc34)c3c4ccccc4cc4sc5ccccc5c34)c2)cc1 ; c1ccc(-c2ccccc2N(c2ccc3c(c2)oc2ccccc23)c2cccc3cc4sc5ccccc5c4cc23)cc1 ; c1cc(N(c2ccc3c(c2)oc2ccccc23)c2ccc3c(c2)oc2ccccc23)c2cc3sc4ccccc4c3cc2c1] 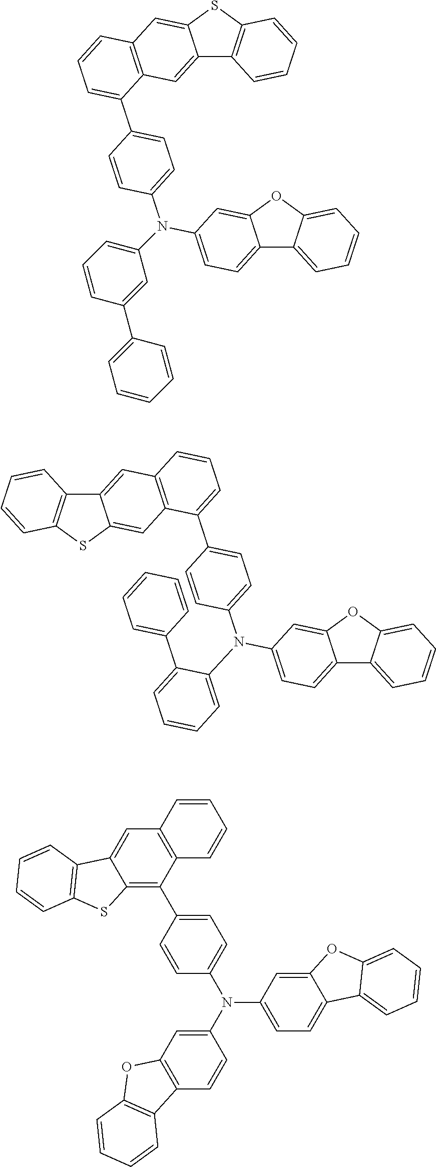 [SMILES: c1ccc(-c2cccc(N(c3ccc(-c4cccc5cc6sc7ccccc7c6cc45)cc3)c3ccc4c(c3)oc3ccccc34)c2)cc1 ; c1ccc(-c2ccccc2N(c2ccc(-c3cccc4cc5c(cc34)sc3ccccc35)cc2)c2ccc3c(c2)oc2ccccc23)cc1 ; c1ccc2c(-c3ccc(N(c4ccc5c(c4)oc4ccccc45)c4ccc5c(c4)oc4ccccc45)cc3)c3sc4ccccc4c3cc2c1] 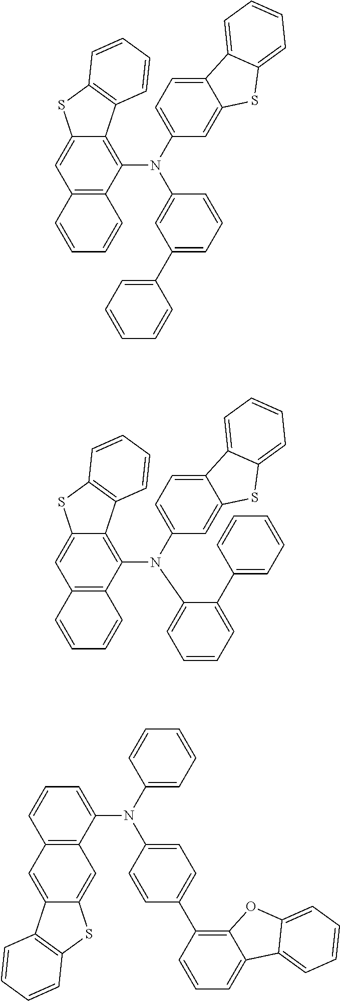 [SMILES: c1ccc(-c2cccc(N(c3ccc4c(c3)sc3ccccc34)c3c4ccccc4cc4sc5ccccc5c34)c2)cc1 ; c1ccc(-c2ccccc2N(c2ccc3c(c2)sc2ccccc23)c2c3ccccc3cc3sc4ccccc4c23)cc1 ; c1ccc(N(c2ccc(-c3cccc4c3oc3ccccc34)cc2)c2cccc3cc4c(cc23)sc2ccccc24)cc1] 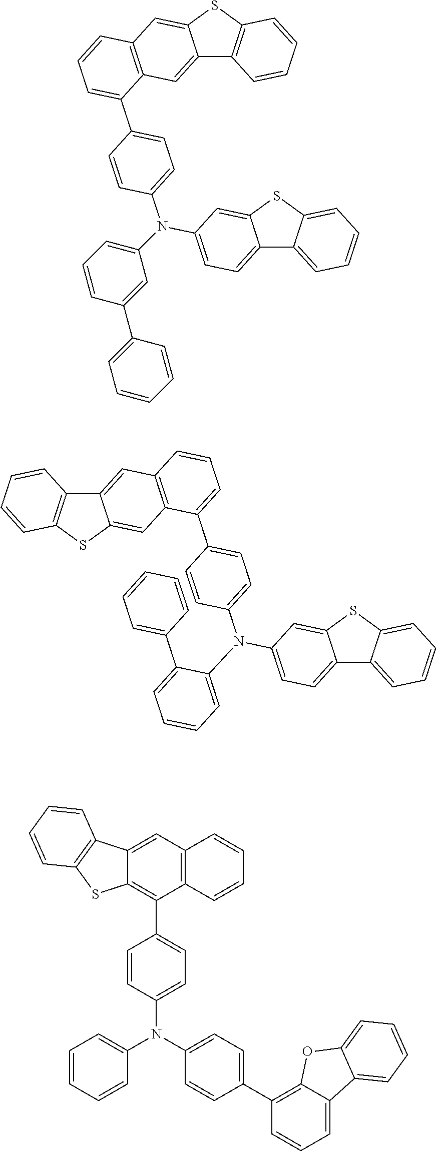 [SMILES: c1ccc(-c2cccc(N(c3ccc(-c4cccc5cc6sc7ccccc7c6cc45)cc3)c3ccc4c(c3)sc3ccccc34)c2)cc1 ; c1ccc(-c2ccccc2N(c2ccc(-c3cccc4cc5c(cc34)sc3ccccc35)cc2)c2ccc3c(c2)sc2ccccc23)cc1 ; c1ccc(N(c2ccc(-c3cccc4c3oc3ccccc34)cc2)c2ccc(-c3c4ccccc4cc4c3sc3ccccc34)cc2)cc1] 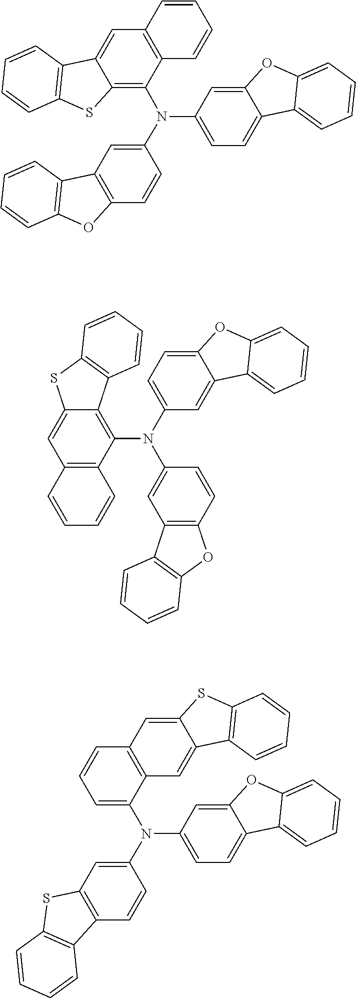 [SMILES: c1ccc2c(N(c3ccc4c(c3)oc3ccccc34)c3ccc4oc5ccccc5c4c3)c3sc4ccccc4c3cc2c1 ; c1ccc2c(N(c3ccc4oc5ccccc5c4c3)c3ccc4oc5ccccc5c4c3)c3c(cc2c1)sc1ccccc13 ; c1cc(N(c2ccc3c(c2)oc2ccccc23)c2ccc3c(c2)sc2ccccc23)c2cc3c(cc2c1)sc1ccccc13] 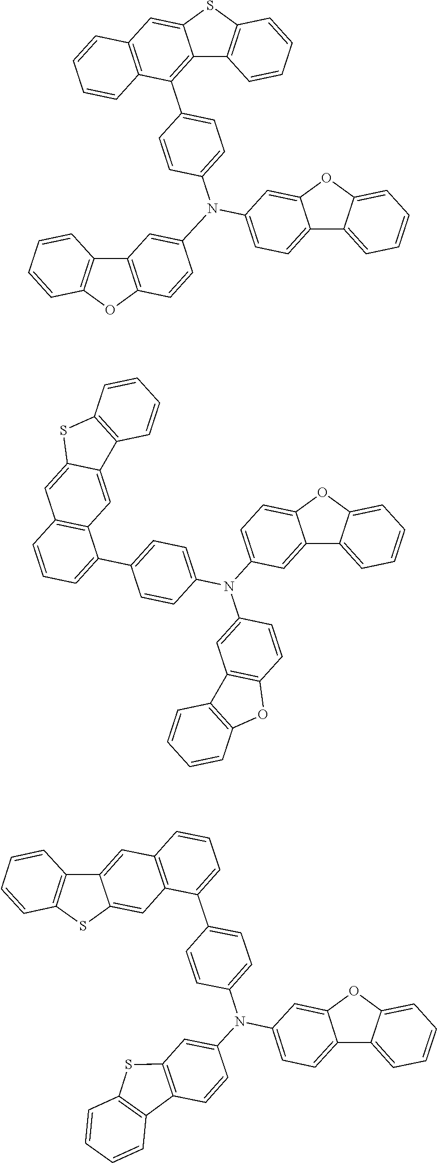 [SMILES: c1ccc2c(-c3ccc(N(c4ccc5c(c4)oc4ccccc45)c4ccc5oc6ccccc6c5c4)cc3)c3c(cc2c1)sc1ccccc13 ; c1cc(-c2ccc(N(c3ccc4oc5ccccc5c4c3)c3ccc4oc5ccccc5c4c3)cc2)c2cc3c(cc2c1)sc1ccccc13 ; c1cc(-c2ccc(N(c3ccc4c(c3)oc3ccccc34)c3ccc4c(c3)sc3ccccc34)cc2)c2cc3sc4ccccc4c3cc2c1] 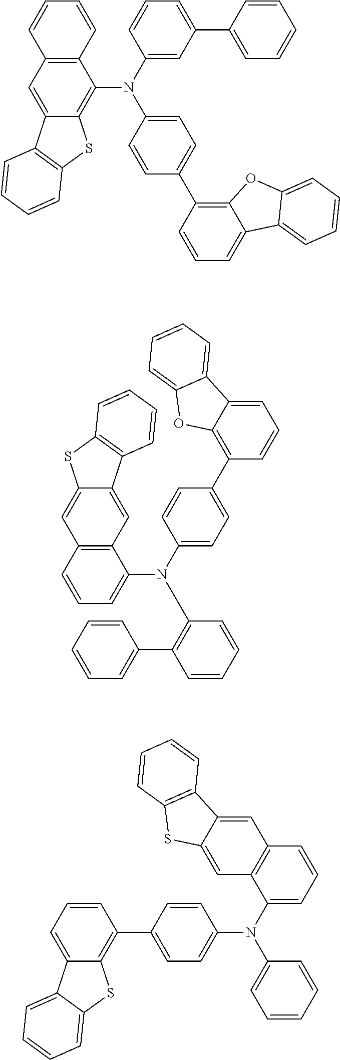 [SMILES: c1ccc(-c2cccc(N(c3ccc(-c4cccc5c4oc4ccccc45)cc3)c3c4ccccc4cc4c3sc3ccccc34)c2)cc1 ; c1ccc(-c2ccccc2N(c2ccc(-c3cccc4c3oc3ccccc34)cc2)c2cccc3cc4sc5ccccc5c4cc23)cc1 ; c1ccc(N(c2ccc(-c3cccc4c3sc3ccccc34)cc2)c2cccc3cc4c(cc23)sc2ccccc24)cc1] 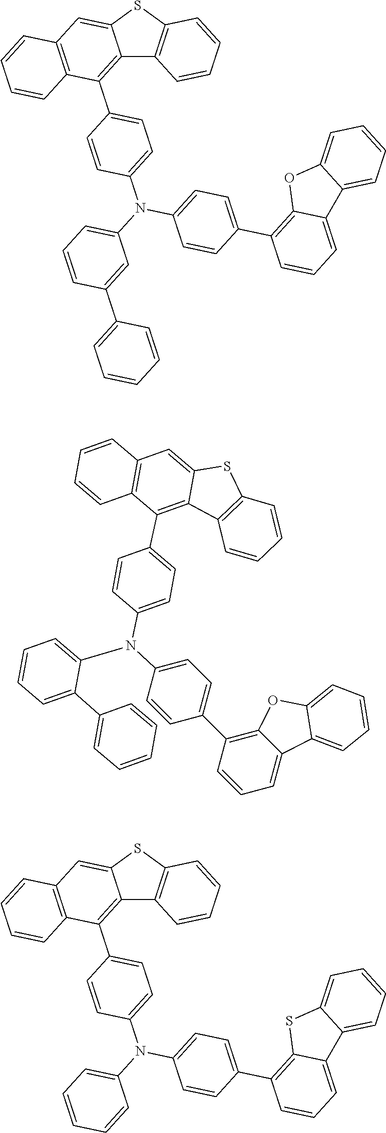 [SMILES: c1ccc(-c2cccc(N(c3ccc(-c4cccc5c4oc4ccccc45)cc3)c3ccc(-c4c5ccccc5cc5sc6ccccc6c45)cc3)c2)cc1 ; c1ccc(-c2ccccc2N(c2ccc(-c3cccc4c3oc3ccccc34)cc2)c2ccc(-c3c4ccccc4cc4sc5ccccc5c34)cc2)cc1 ; c1ccc(N(c2ccc(-c3cccc4c3sc3ccccc34)cc2)c2ccc(-c3c4ccccc4cc4sc5ccccc5c34)cc2)cc1] 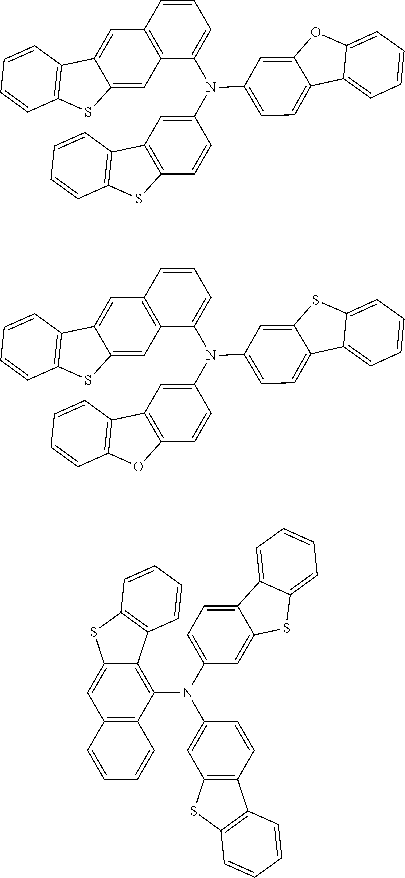 [SMILES: c1cc(N(c2ccc3c(c2)oc2ccccc23)c2ccc3sc4ccccc4c3c2)c2cc3sc4ccccc4c3cc2c1 ; c1cc(N(c2ccc3c(c2)sc2ccccc23)c2ccc3oc4ccccc4c3c2)c2cc3sc4ccccc4c3cc2c1 ; c1ccc2c(N(c3ccc4c(c3)sc3ccccc34)c3ccc4c(c3)sc3ccccc34)c3c(cc2c1)sc1ccccc13] 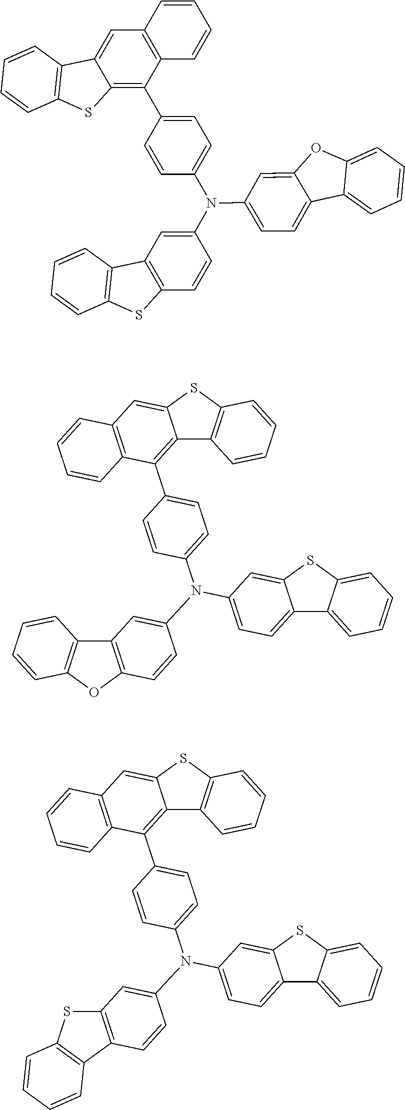 [SMILES: c1ccc2c(-c3ccc(N(c4ccc5c(c4)oc4ccccc45)c4ccc5sc6ccccc6c5c4)cc3)c3sc4ccccc4c3cc2c1 ; c1ccc2c(-c3ccc(N(c4ccc5c(c4)sc4ccccc45)c4ccc5oc6ccccc6c5c4)cc3)c3c(cc2c1)sc1ccccc13 ; c1ccc2c(-c3ccc(N(c4ccc5c(c4)sc4ccccc45)c4ccc5c(c4)sc4ccccc45)cc3)c3c(cc2c1)sc1ccccc13] 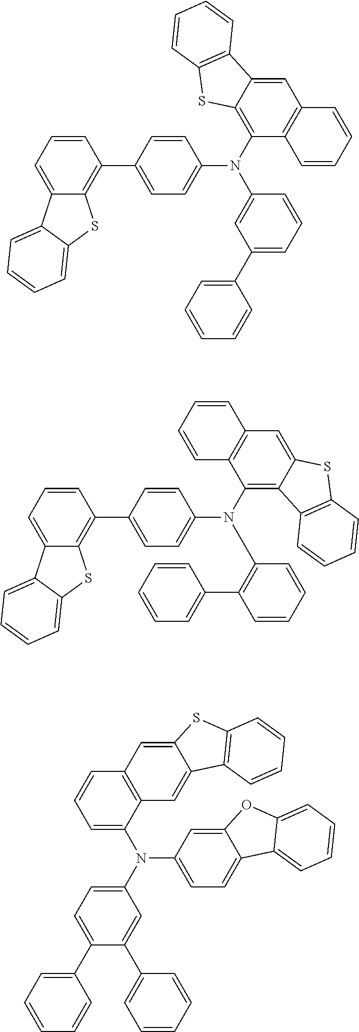 [SMILES: c1ccc(-c2cccc(N(c3ccc(-c4cccc5c4sc4ccccc45)cc3)c3c4ccccc4cc4c3sc3ccccc34)c2)cc1 ; c1ccc(-c2ccccc2N(c2ccc(-c3cccc4c3sc3ccccc34)cc2)c2c3ccccc3cc3sc4ccccc4c23)cc1 ; c1ccc(-c2ccc(N(c3ccc4c(c3)oc3ccccc34)c3cccc4cc5sc6ccccc6c5cc34)cc2-c2ccccc2)cc1] 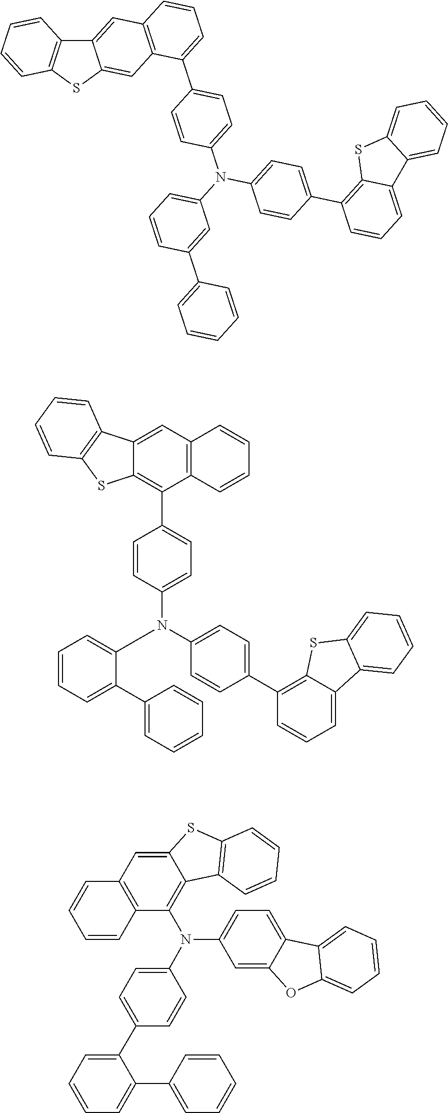 [SMILES: c1ccc(-c2cccc(N(c3ccc(-c4cccc5cc6c(cc45)sc4ccccc46)cc3)c3ccc(-c4cccc5c4sc4ccccc45)cc3)c2)cc1 ; c1ccc(-c2ccccc2N(c2ccc(-c3cccc4c3sc3ccccc34)cc2)c2ccc(-c3c4ccccc4cc4c3sc3ccccc34)cc2)cc1 ; c1ccc(-c2ccccc2-c2ccc(N(c3ccc4c(c3)oc3ccccc34)c3c4ccccc4cc4sc5ccccc5c34)cc2)cc1] 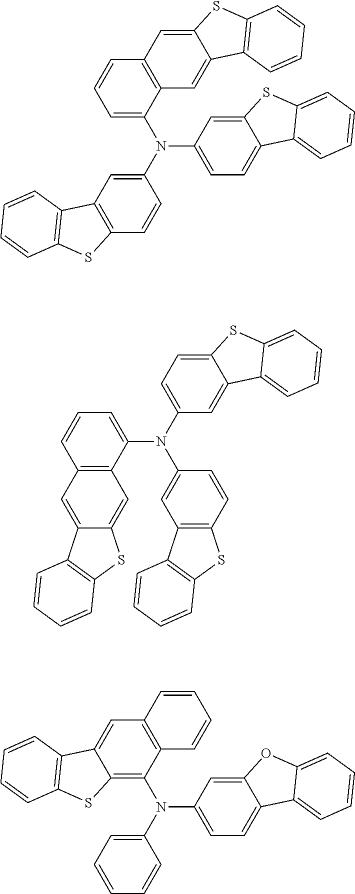 [SMILES: c1cc(N(c2ccc3c(c2)sc2ccccc23)c2ccc3sc4ccccc4c3c2)c2cc3c(cc2c1)sc1ccccc13 ; c1cc(N(c2ccc3sc4ccccc4c3c2)c2ccc3sc4ccccc4c3c2)c2cc3sc4ccccc4c3cc2c1 ; c1ccc(N(c2ccc3c(c2)oc2ccccc23)c2c3ccccc3cc3c2sc2ccccc23)cc1] 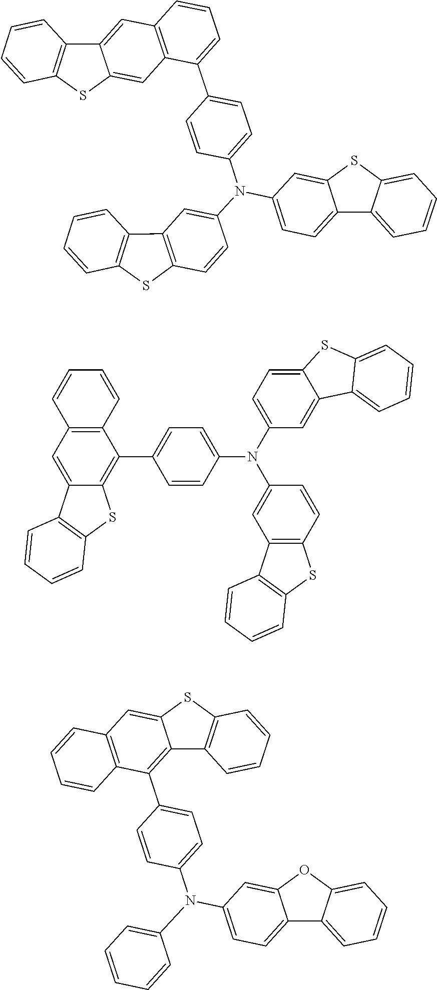 [SMILES: c1cc(-c2ccc(N(c3ccc4c(c3)sc3ccccc34)c3ccc4sc5ccccc5c4c3)cc2)c2cc3sc4ccccc4c3cc2c1 ; c1ccc2c(-c3ccc(N(c4ccc5sc6ccccc6c5c4)c4ccc5sc6ccccc6c5c4)cc3)c3sc4ccccc4c3cc2c1 ; c1ccc(N(c2ccc(-c3c4ccccc4cc4sc5ccccc5c34)cc2)c2ccc3c(c2)oc2ccccc23)cc1] 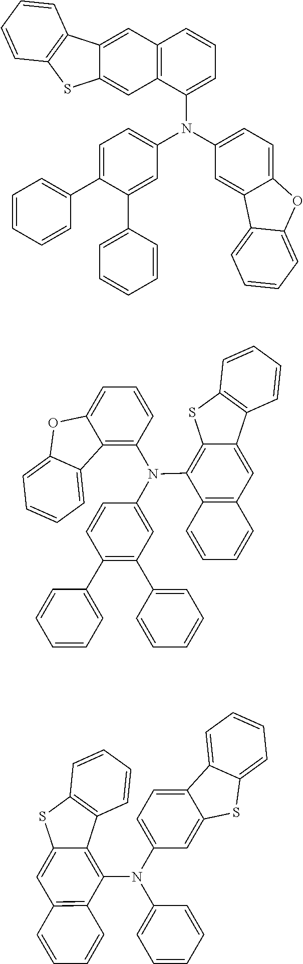 [SMILES: c1ccc(-c2ccc(N(c3ccc4oc5ccccc5c4c3)c3cccc4cc5c(cc34)sc3ccccc35)cc2-c2ccccc2)cc1 ; c1ccc(-c2ccc(N(c3c4ccccc4cc4c3sc3ccccc34)c3cccc4oc5ccccc5c34)cc2-c2ccccc2)cc1 ; c1ccc(N(c2ccc3c(c2)sc2ccccc23)c2c3ccccc3cc3sc4ccccc4c23)cc1] 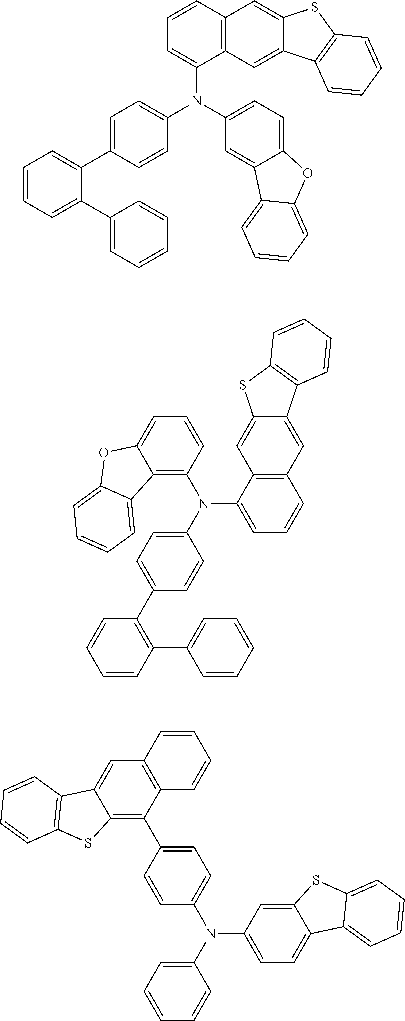 [SMILES: c1ccc(-c2ccccc2-c2ccc(N(c3ccc4oc5ccccc5c4c3)c3cccc4cc5sc6ccccc6c5cc34)cc2)cc1 ; c1ccc(-c2ccccc2-c2ccc(N(c3cccc4cc5c(cc34)sc3ccccc35)c3cccc4oc5ccccc5c34)cc2)cc1 ; c1ccc(N(c2ccc(-c3c4ccccc4cc4c3sc3ccccc34)cc2)c2ccc3c(c2)sc2ccccc23)cc1] 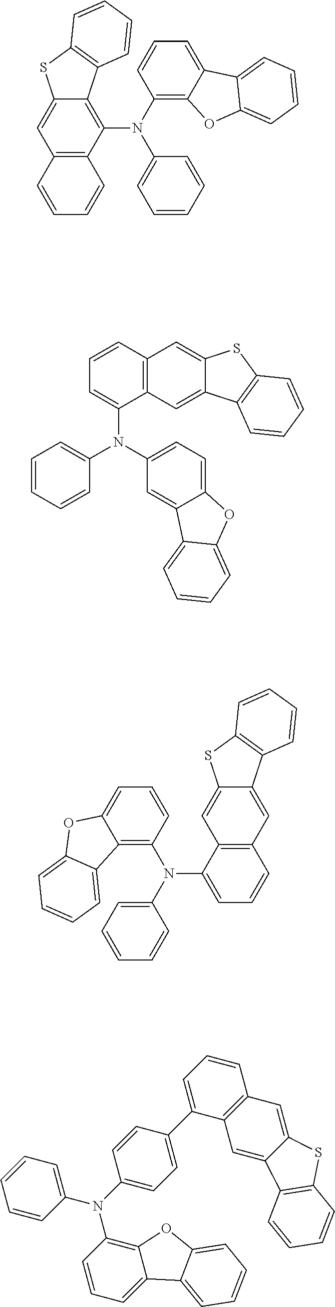 [SMILES: c1ccc(N(c2cccc3c2oc2ccccc23)c2c3ccccc3cc3sc4ccccc4c23)cc1 ; c1ccc(N(c2ccc3oc4ccccc4c3c2)c2cccc3cc4sc5ccccc5c4cc23)cc1 ; c1ccc(N(c2cccc3cc4c(cc23)sc2ccccc24)c2cccc3oc4ccccc4c23)cc1 ; c1ccc(N(c2ccc(-c3cccc4cc5sc6ccccc6c5cc34)cc2)c2cccc3c2oc2ccccc23)cc1] 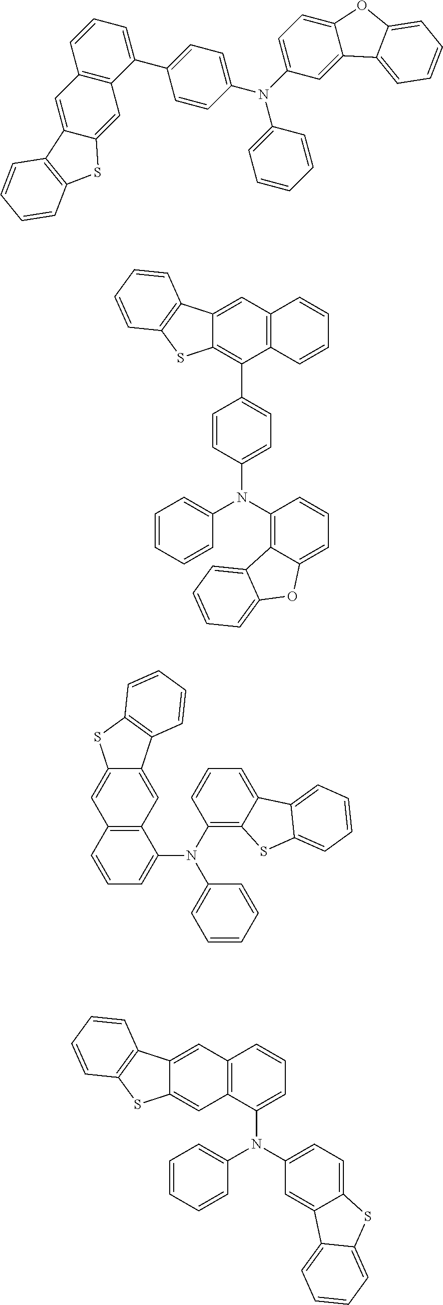 [SMILES: c1ccc(N(c2ccc(-c3cccc4cc5c(cc34)sc3ccccc35)cc2)c2ccc3oc4ccccc4c3c2)cc1 ; c1ccc(N(c2ccc(-c3c4ccccc4cc4c3sc3ccccc34)cc2)c2cccc3oc4ccccc4c23)cc1 ; c1ccc(N(c2cccc3cc4sc5ccccc5c4cc23)c2cccc3c2sc2ccccc23)cc1 ; c1ccc(N(c2ccc3sc4ccccc4c3c2)c2cccc3cc4c(cc23)sc2ccccc24)cc1] 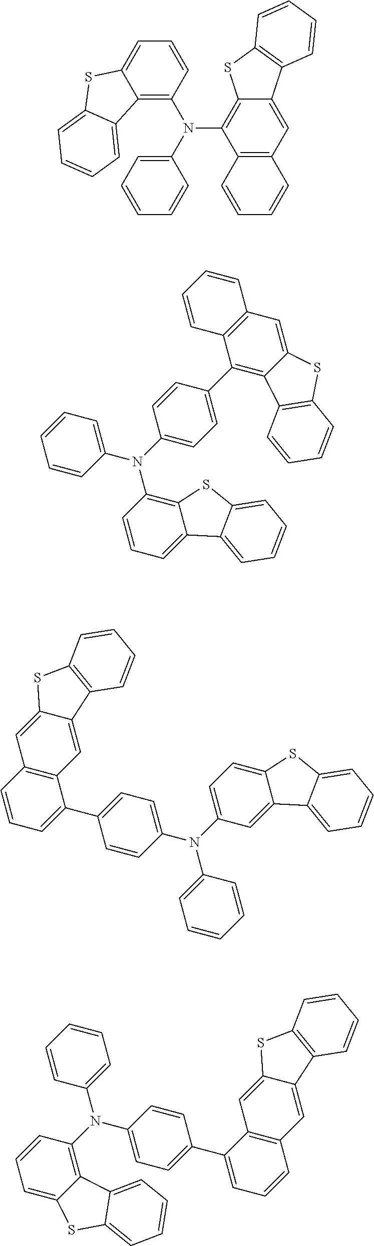 [SMILES: c1ccc(N(c2c3ccccc3cc3c2sc2ccccc23)c2cccc3sc4ccccc4c23)cc1 ; c1ccc(N(c2ccc(-c3c4ccccc4cc4sc5ccccc5c34)cc2)c2cccc3c2sc2ccccc23)cc1 ; c1ccc(N(c2ccc(-c3cccc4cc5sc6ccccc6c5cc34)cc2)c2ccc3sc4ccccc4c3c2)cc1 ; c1ccc(N(c2ccc(-c3cccc4cc5c(cc34)sc3ccccc35)cc2)c2cccc3sc4ccccc4c23)cc1] 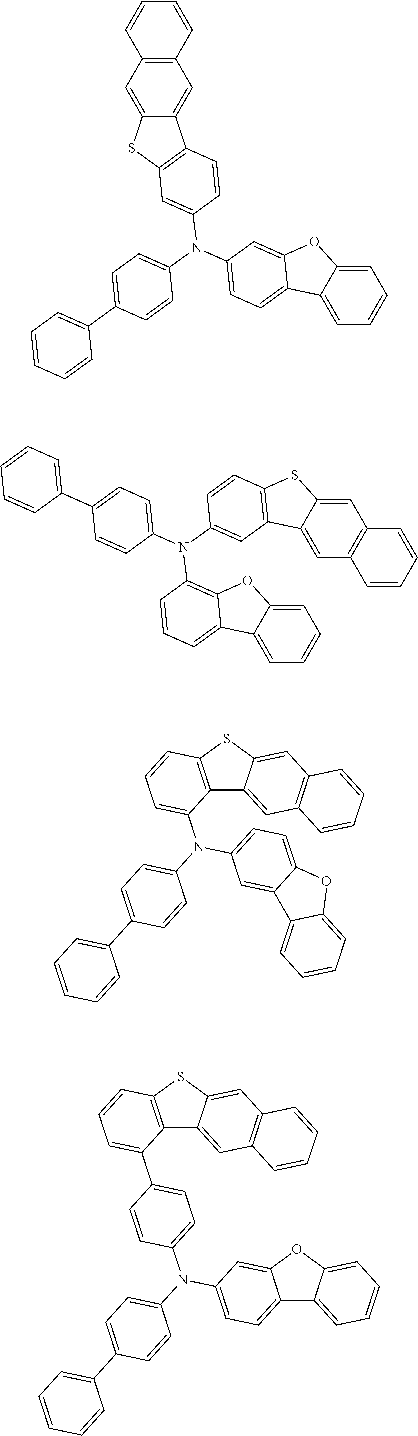 [SMILES: c1ccc(-c2ccc(N(c3ccc4c(c3)oc3ccccc34)c3ccc4c(c3)sc3cc5ccccc5cc34)cc2)cc1 ; c1ccc(-c2ccc(N(c3ccc4sc5cc6ccccc6cc5c4c3)c3cccc4c3oc3ccccc34)cc2)cc1 ; c1ccc(-c2ccc(N(c3ccc4oc5ccccc5c4c3)c3cccc4sc5cc6ccccc6cc5c34)cc2)cc1 ; c1ccc(-c2ccc(N(c3ccc(-c4cccc5sc6cc7ccccc7cc6c45)cc3)c3ccc4c(c3)oc3ccccc34)cc2)cc1] 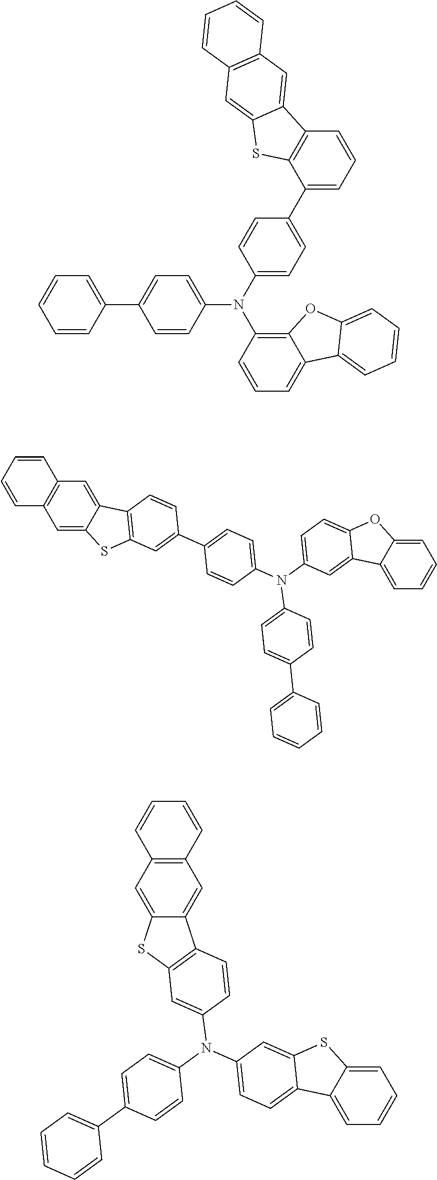 [SMILES: c1ccc(-c2ccc(N(c3ccc(-c4cccc5c4sc4cc6ccccc6cc45)cc3)c3cccc4c3oc3ccccc34)cc2)cc1 ; c1ccc(-c2ccc(N(c3ccc(-c4ccc5c(c4)sc4cc6ccccc6cc45)cc3)c3ccc4oc5ccccc5c4c3)cc2)cc1 ; c1ccc(-c2ccc(N(c3ccc4c(c3)sc3ccccc34)c3ccc4c(c3)sc3cc5ccccc5cc34)cc2)cc1] 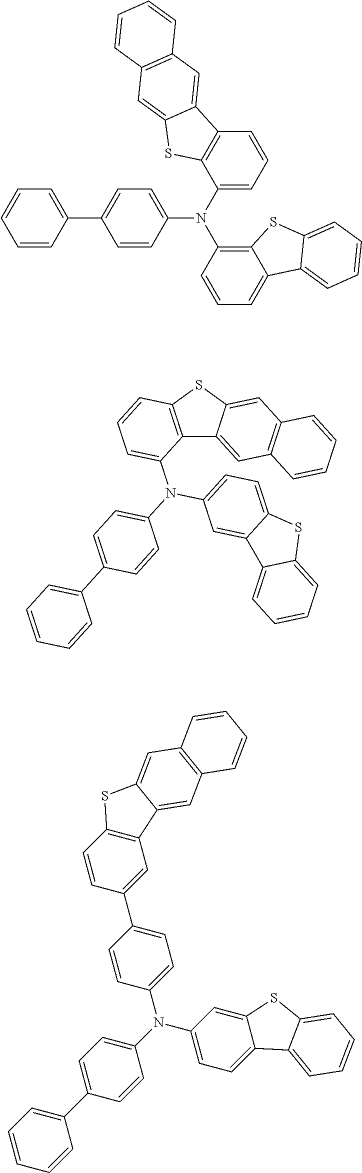 [SMILES: c1ccc(-c2ccc(N(c3cccc4c3sc3ccccc34)c3cccc4c3sc3cc5ccccc5cc34)cc2)cc1 ; c1ccc(-c2ccc(N(c3ccc4sc5ccccc5c4c3)c3cccc4sc5cc6ccccc6cc5c34)cc2)cc1 ; c1ccc(-c2ccc(N(c3ccc(-c4ccc5sc6cc7ccccc7cc6c5c4)cc3)c3ccc4c(c3)sc3ccccc34)cc2)cc1] 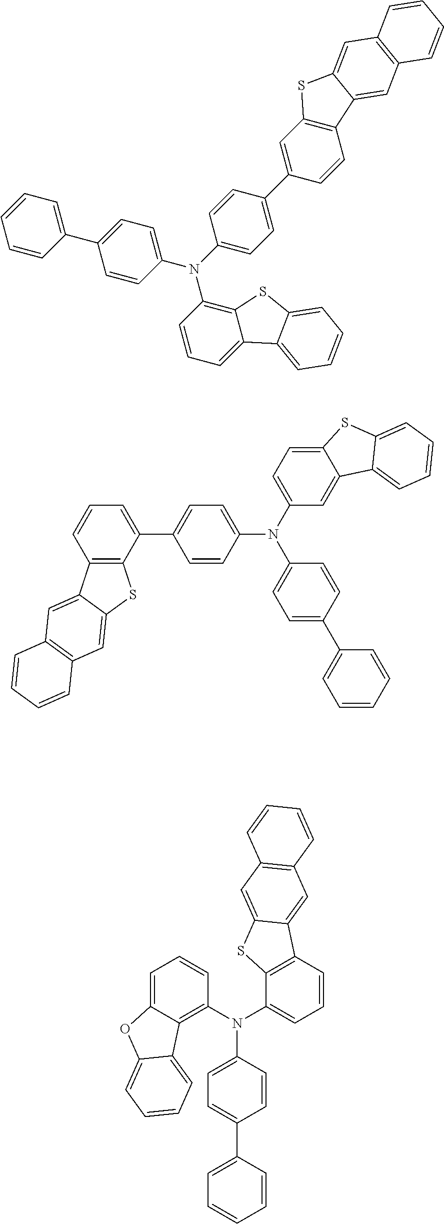 [SMILES: c1ccc(-c2ccc(N(c3ccc(-c4ccc5c(c4)sc4cc6ccccc6cc45)cc3)c3cccc4c3sc3ccccc34)cc2)cc1 ; c1ccc(-c2ccc(N(c3ccc(-c4cccc5c4sc4cc6ccccc6cc45)cc3)c3ccc4sc5ccccc5c4c3)cc2)cc1 ; c1ccc(-c2ccc(N(c3cccc4c3sc3cc5ccccc5cc34)c3cccc4oc5ccccc5c34)cc2)cc1] 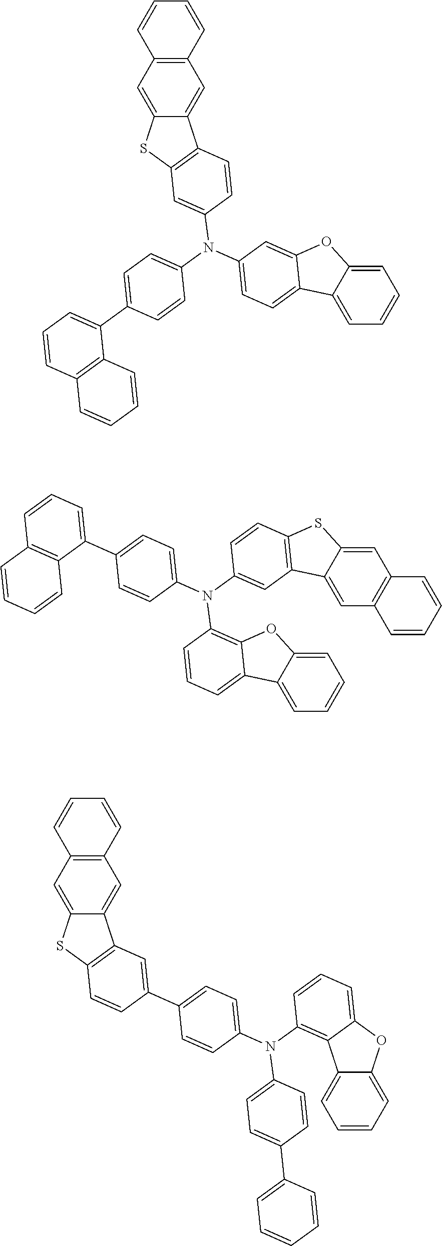 [SMILES: c1ccc2cc3c(cc2c1)sc1cc(N(c2ccc(-c4cccc5ccccc45)cc2)c2ccc4c(c2)oc2ccccc24)ccc13 ; c1ccc2cc3c(cc2c1)sc1ccc(N(c2ccc(-c4cccc5ccccc45)cc2)c2cccc4c2oc2ccccc24)cc13 ; c1ccc(-c2ccc(N(c3ccc(-c4ccc5sc6cc7ccccc7cc6c5c4)cc3)c3cccc4oc5ccccc5c34)cc2)cc1] 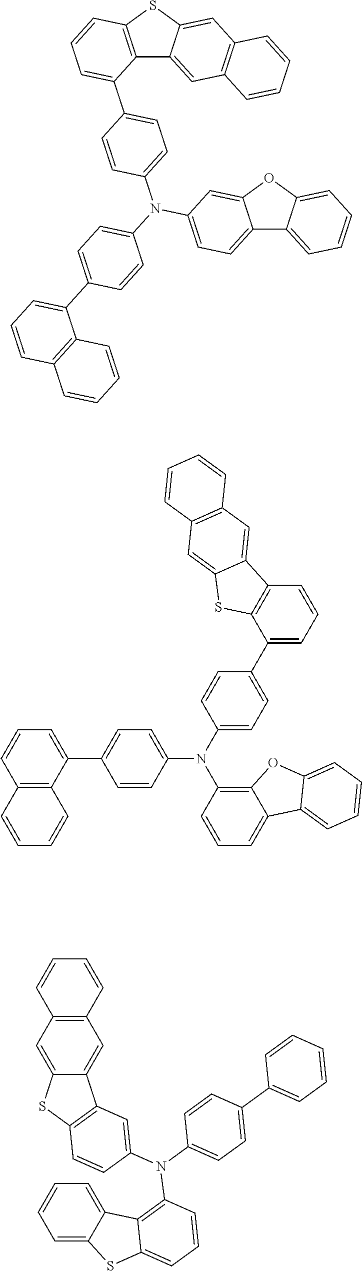 [SMILES: c1ccc2cc3c(cc2c1)sc1cccc(-c2ccc(N(c4ccc(-c5cccc6ccccc56)cc4)c4ccc5c(c4)oc4ccccc45)cc2)c13 ; c1ccc2cc3c(cc2c1)sc1c(-c2ccc(N(c4ccc(-c5cccc6ccccc56)cc4)c4cccc5c4oc4ccccc45)cc2)cccc13 ; c1ccc(-c2ccc(N(c3ccc4sc5cc6ccccc6cc5c4c3)c3cccc4sc5ccccc5c34)cc2)cc1] 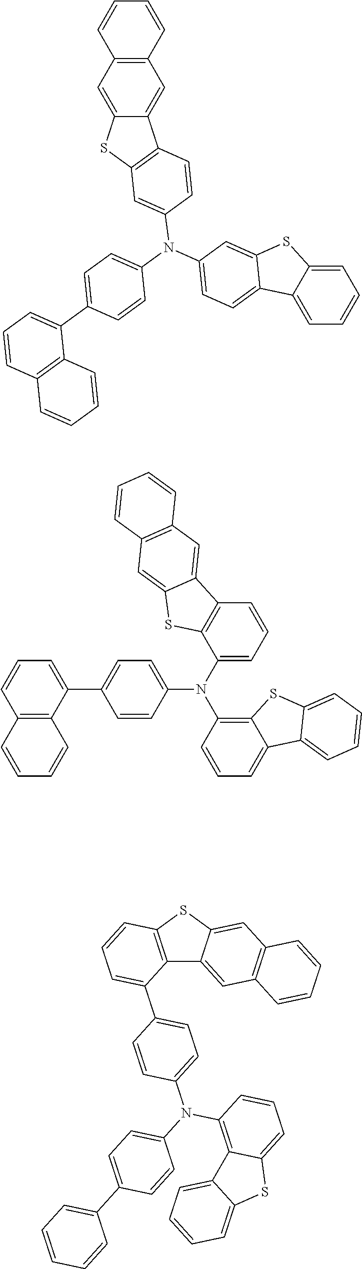 [SMILES: c1ccc2cc3c(cc2c1)sc1cc(N(c2ccc(-c4cccc5ccccc45)cc2)c2ccc4c(c2)sc2ccccc24)ccc13 ; c1ccc2cc3c(cc2c1)sc1c(N(c2ccc(-c4cccc5ccccc45)cc2)c2cccc4c2sc2ccccc24)cccc13 ; c1ccc(-c2ccc(N(c3ccc(-c4cccc5sc6cc7ccccc7cc6c45)cc3)c3cccc4sc5ccccc5c34)cc2)cc1] 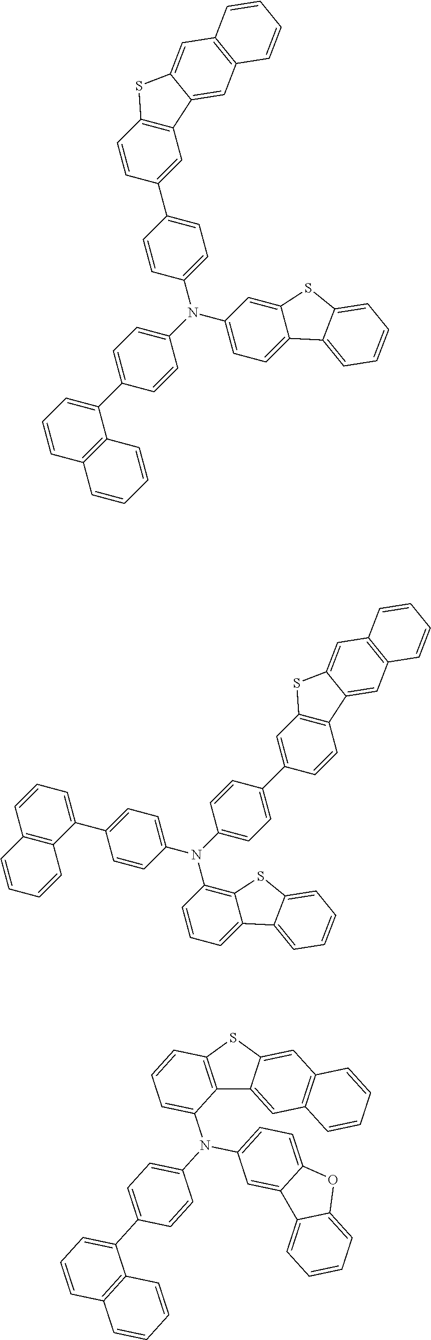 [SMILES: c1ccc2cc3c(cc2c1)sc1ccc(-c2ccc(N(c4ccc(-c5cccc6ccccc56)cc4)c4ccc5c(c4)sc4ccccc45)cc2)cc13 ; c1ccc2cc3c(cc2c1)sc1cc(-c2ccc(N(c4ccc(-c5cccc6ccccc56)cc4)c4cccc5c4sc4ccccc45)cc2)ccc13 ; c1ccc2cc3c(cc2c1)sc1cccc(N(c2ccc(-c4cccc5ccccc45)cc2)c2ccc4oc5ccccc5c4c2)c13] 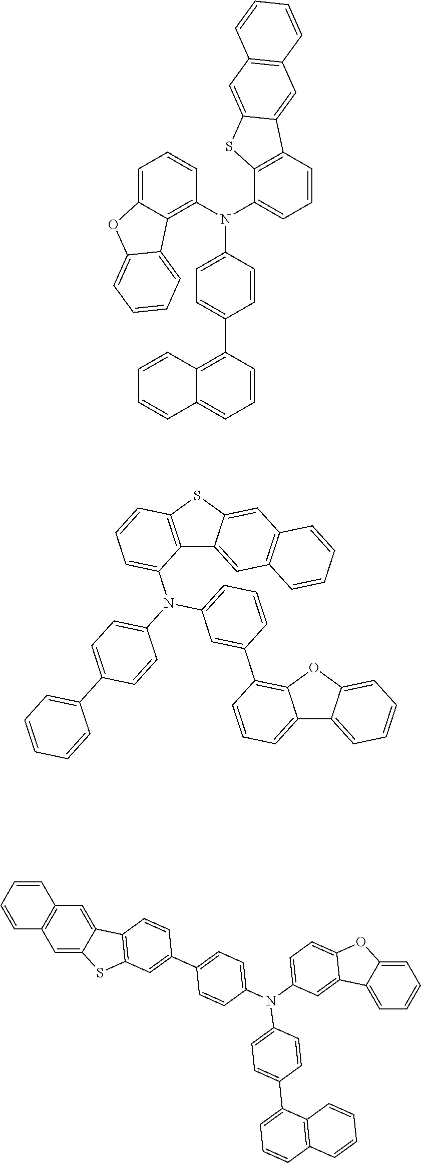 [SMILES: c1ccc2cc3c(cc2c1)sc1c(N(c2ccc(-c4cccc5ccccc45)cc2)c2cccc4oc5ccccc5c24)cccc13 ; c1ccc(-c2ccc(N(c3cccc(-c4cccc5c4oc4ccccc45)c3)c3cccc4sc5cc6ccccc6cc5c34)cc2)cc1 ; c1ccc2cc3c(cc2c1)sc1cc(-c2ccc(N(c4ccc(-c5cccc6ccccc56)cc4)c4ccc5oc6ccccc6c5c4)cc2)ccc13] 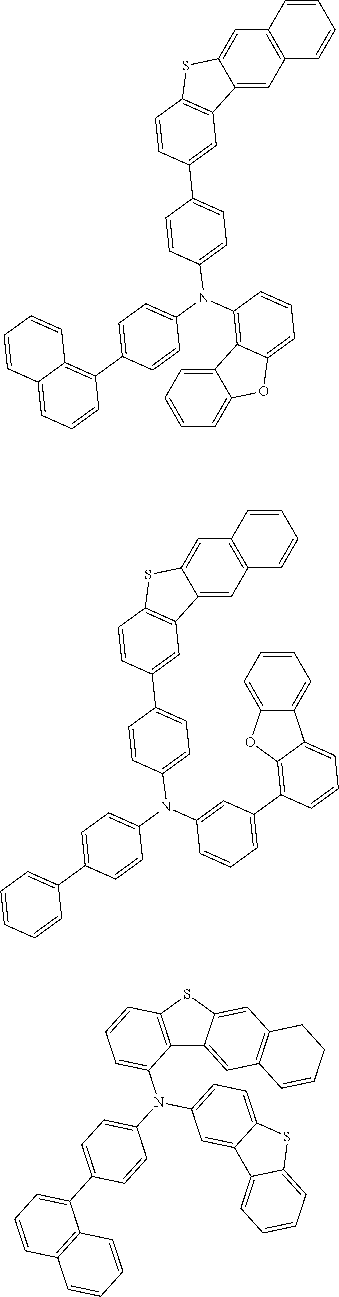 [SMILES: c1ccc2cc3c(cc2c1)sc1ccc(-c2ccc(N(c4ccc(-c5cccc6ccccc56)cc4)c4cccc5oc6ccccc6c45)cc2)cc13 ; c1ccc(-c2ccc(N(c3ccc(-c4ccc5sc6cc7ccccc7cc6c5c4)cc3)c3cccc(-c4cccc5c4oc4ccccc45)c3)cc2)cc1 ; C1=Cc2cc3c(cc2CC1)sc1cccc(N(c2ccc(-c4cccc5ccccc45)cc2)c2ccc4sc5ccccc5c4c2)c13] 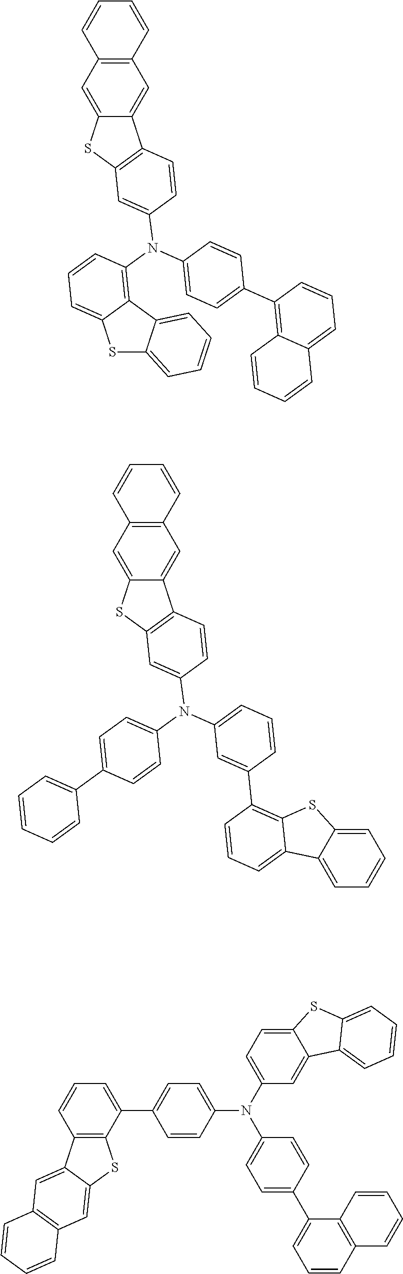 [SMILES: c1ccc2cc3c(cc2c1)sc1cc(N(c2ccc(-c4cccc5ccccc45)cc2)c2cccc4sc5ccccc5c24)ccc13 ; c1ccc(-c2ccc(N(c3cccc(-c4cccc5c4sc4ccccc45)c3)c3ccc4c(c3)sc3cc5ccccc5cc34)cc2)cc1 ; c1ccc2cc3c(cc2c1)sc1c(-c2ccc(N(c4ccc(-c5cccc6ccccc56)cc4)c4ccc5sc6ccccc6c5c4)cc2)cccc13] 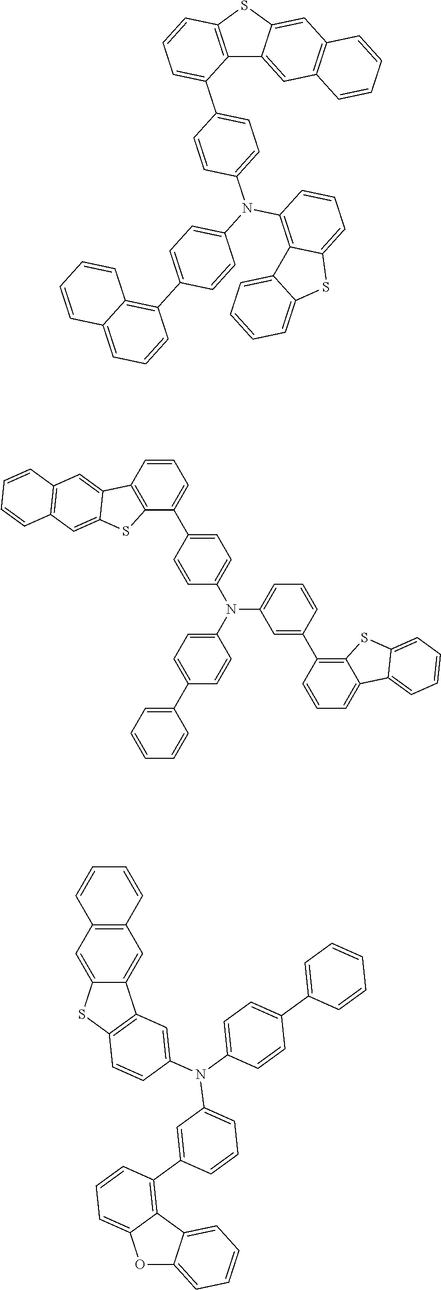 [SMILES: c1ccc2cc3c(cc2c1)sc1cccc(-c2ccc(N(c4ccc(-c5cccc6ccccc56)cc4)c4cccc5sc6ccccc6c45)cc2)c13 ; c1ccc(-c2ccc(N(c3ccc(-c4cccc5c4sc4cc6ccccc6cc45)cc3)c3cccc(-c4cccc5c4sc4ccccc45)c3)cc2)cc1 ; c1ccc(-c2ccc(N(c3cccc(-c4cccc5oc6ccccc6c45)c3)c3ccc4sc5cc6ccccc6cc5c4c3)cc2)cc1] 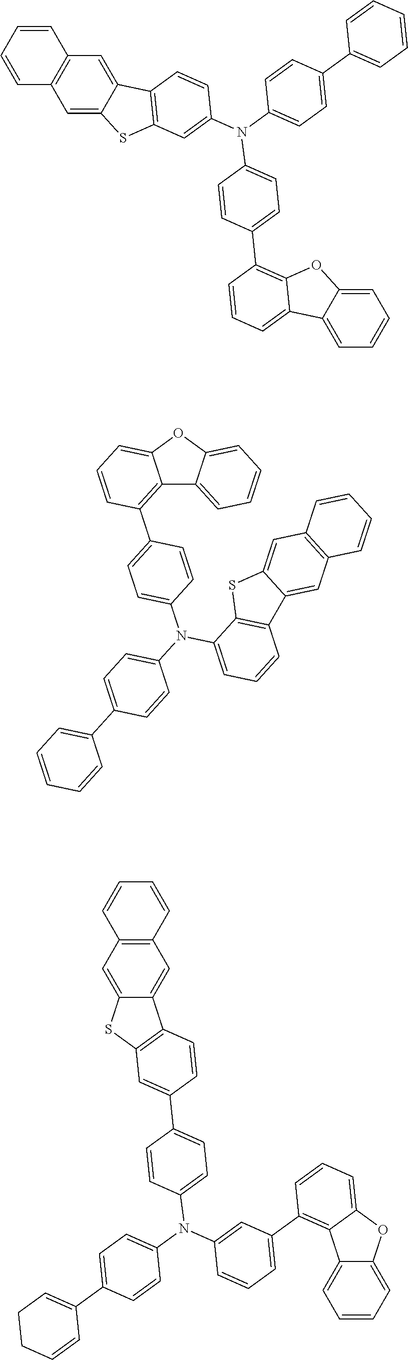 [SMILES: c1ccc(-c2ccc(N(c3ccc(-c4cccc5c4oc4ccccc45)cc3)c3ccc4c(c3)sc3cc5ccccc5cc34)cc2)cc1 ; c1ccc(-c2ccc(N(c3ccc(-c4cccc5oc6ccccc6c45)cc3)c3cccc4c3sc3cc5ccccc5cc34)cc2)cc1 ; C1=CC(c2ccc(N(c3ccc(-c4ccc5c(c4)sc4cc6ccccc6cc45)cc3)c3cccc(-c4cccc5oc6ccccc6c45)c3)cc2)=CCC1] 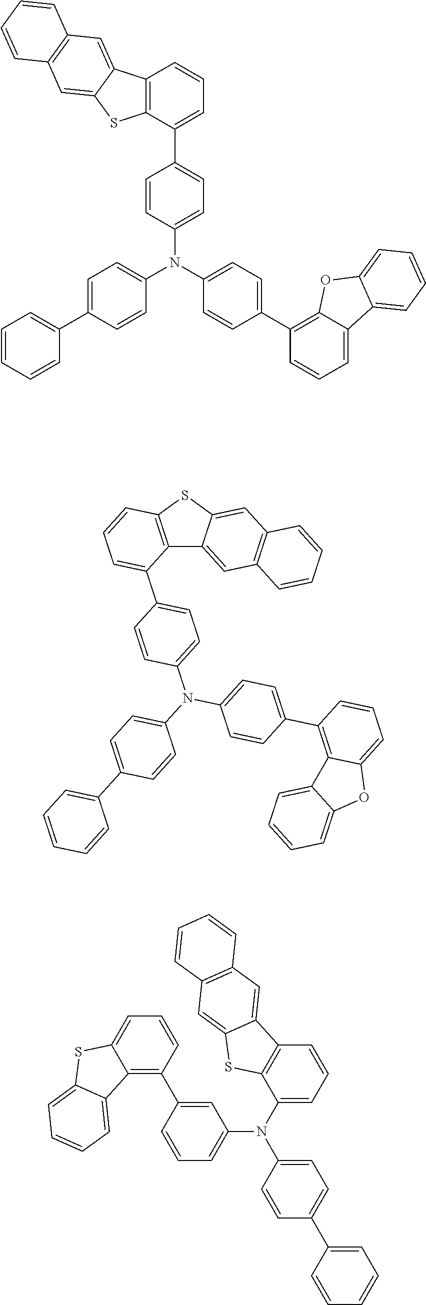 [SMILES: c1ccc(-c2ccc(N(c3ccc(-c4cccc5c4oc4ccccc45)cc3)c3ccc(-c4cccc5c4sc4cc6ccccc6cc45)cc3)cc2)cc1 ; c1ccc(-c2ccc(N(c3ccc(-c4cccc5oc6ccccc6c45)cc3)c3ccc(-c4cccc5sc6cc7ccccc7cc6c45)cc3)cc2)cc1 ; c1ccc(-c2ccc(N(c3cccc(-c4cccc5sc6ccccc6c45)c3)c3cccc4c3sc3cc5ccccc5cc34)cc2)cc1] 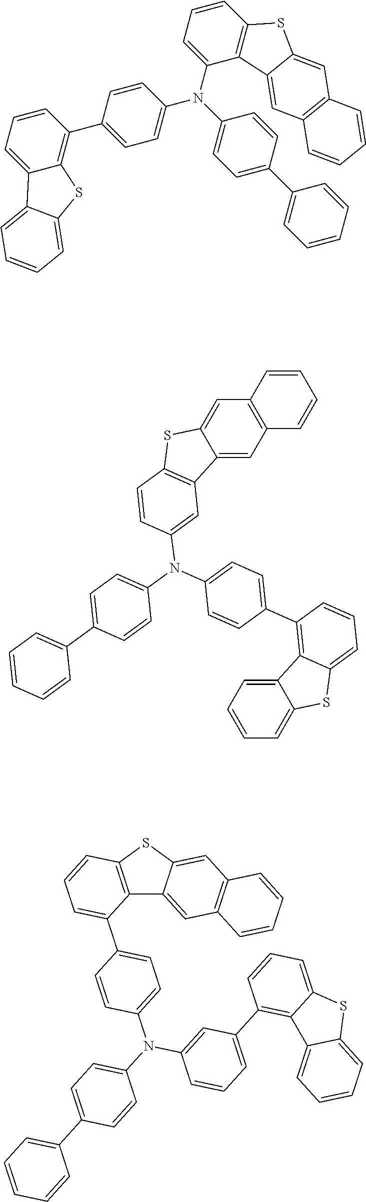 [SMILES: c1ccc(-c2ccc(N(c3ccc(-c4cccc5c4sc4ccccc45)cc3)c3cccc4sc5cc6ccccc6cc5c34)cc2)cc1 ; c1ccc(-c2ccc(N(c3ccc(-c4cccc5sc6ccccc6c45)cc3)c3ccc4sc5cc6ccccc6cc5c4c3)cc2)cc1 ; c1ccc(-c2ccc(N(c3ccc(-c4cccc5sc6cc7ccccc7cc6c45)cc3)c3cccc(-c4cccc5sc6ccccc6c45)c3)cc2)cc1] 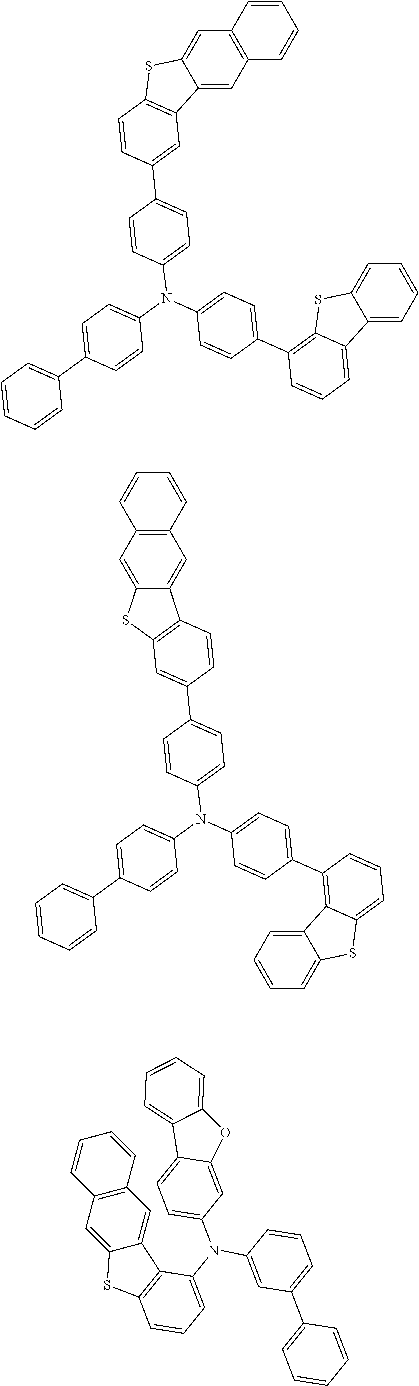 [SMILES: c1ccc(-c2ccc(N(c3ccc(-c4ccc5sc6cc7ccccc7cc6c5c4)cc3)c3ccc(-c4cccc5c4sc4ccccc45)cc3)cc2)cc1 ; c1ccc(-c2ccc(N(c3ccc(-c4ccc5c(c4)sc4cc6ccccc6cc45)cc3)c3ccc(-c4cccc5sc6ccccc6c45)cc3)cc2)cc1 ; c1ccc(-c2cccc(N(c3ccc4c(c3)oc3ccccc34)c3cccc4sc5cc6ccccc6cc5c34)c2)cc1] 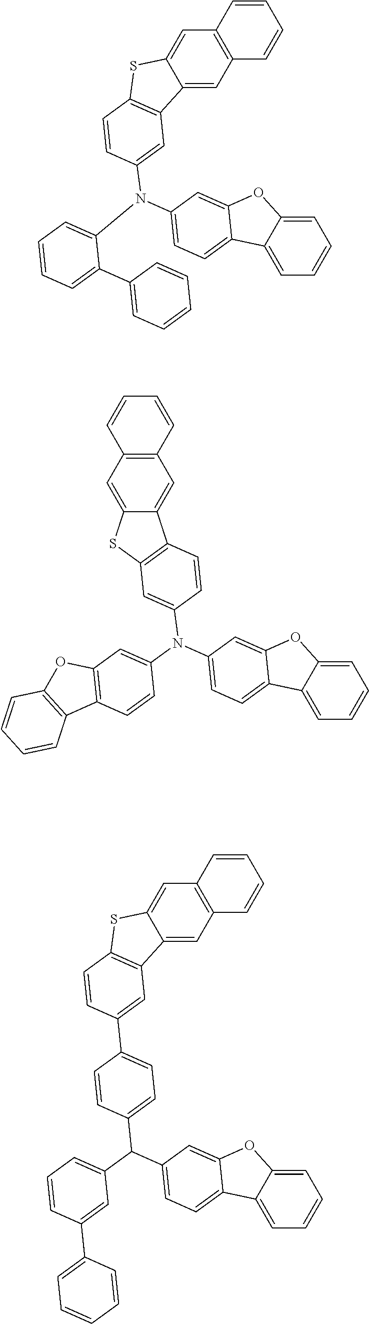 [SMILES: c1ccc(-c2ccccc2N(c2ccc3c(c2)oc2ccccc23)c2ccc3sc4cc5ccccc5cc4c3c2)cc1 ; c1ccc2cc3c(cc2c1)sc1cc(N(c2ccc4c(c2)oc2ccccc24)c2ccc4c(c2)oc2ccccc24)ccc13 ; c1ccc(-c2cccc(C(c3ccc(-c4ccc5sc6cc7ccccc7cc6c5c4)cc3)c3ccc4c(c3)oc3ccccc34)c2)cc1] 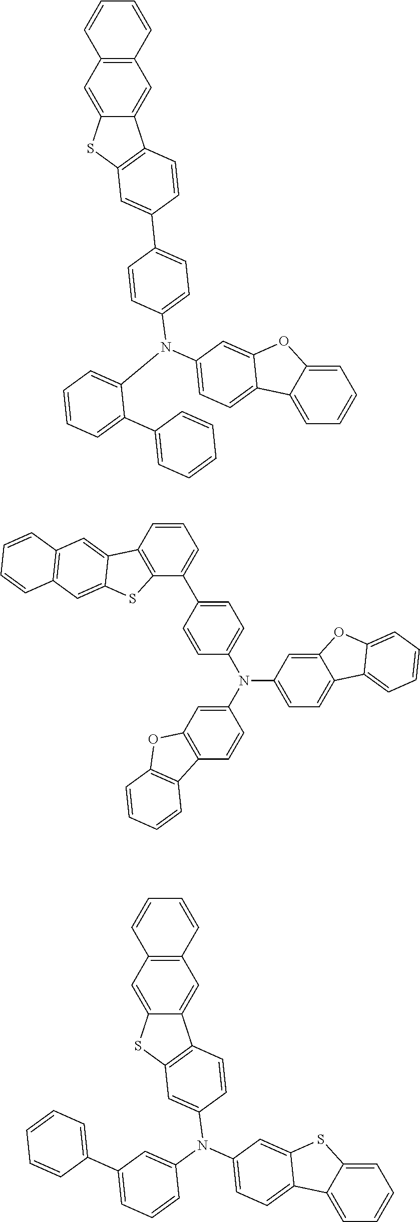 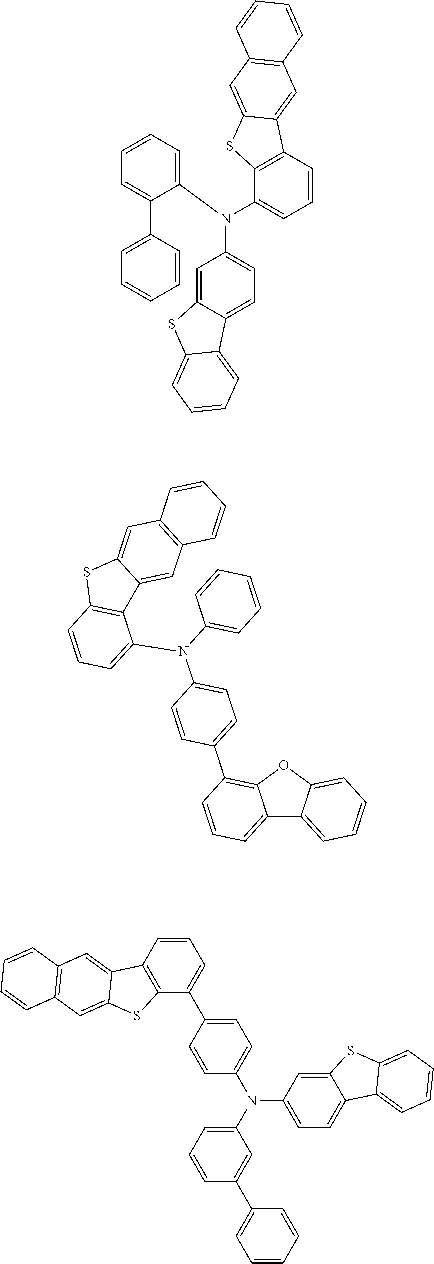 [SMILES: c1ccc(-c2ccccc2N(c2ccc3c(c2)sc2ccccc23)c2cccc3c2sc2cc4ccccc4cc23)cc1 ; c1ccc(N(c2ccc(-c3cccc4c3oc3ccccc34)cc2)c2cccc3sc4cc5ccccc5cc4c23)cc1 ; c1ccc(-c2cccc(N(c3ccc(-c4cccc5c4sc4cc6ccccc6cc45)cc3)c3ccc4c(c3)sc3ccccc34)c2)cc1] 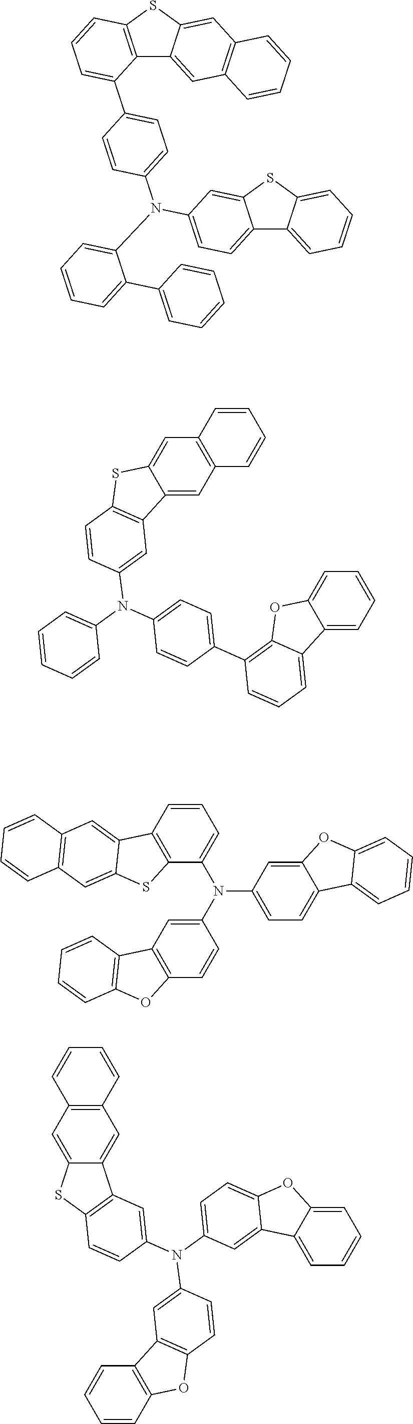 [SMILES: c1ccc(-c2ccccc2N(c2ccc(-c3cccc4sc5cc6ccccc6cc5c34)cc2)c2ccc3c(c2)sc2ccccc23)cc1 ; c1ccc(N(c2ccc(-c3cccc4c3oc3ccccc34)cc2)c2ccc3sc4cc5ccccc5cc4c3c2)cc1 ; c1ccc2cc3c(cc2c1)sc1c(N(c2ccc4c(c2)oc2ccccc24)c2ccc4oc5ccccc5c4c2)cccc13 ; c1ccc2cc3c(cc2c1)sc1ccc(N(c2ccc4oc5ccccc5c4c2)c2ccc4oc5ccccc5c4c2)cc13] 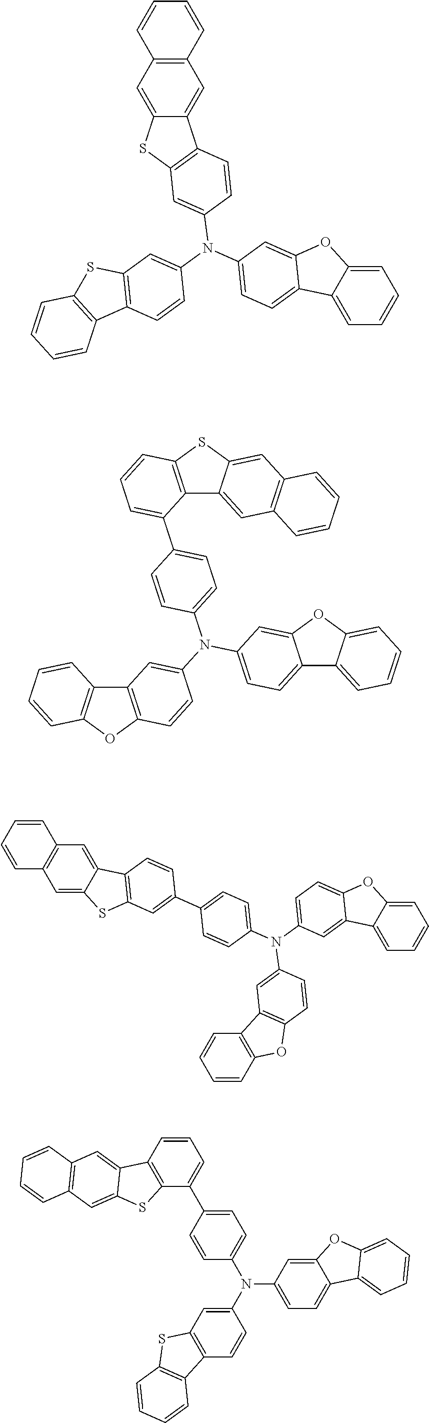 [SMILES: c1ccc2cc3c(cc2c1)sc1cc(N(c2ccc4c(c2)oc2ccccc24)c2ccc4c(c2)sc2ccccc24)ccc13 ; c1ccc2cc3c(cc2c1)sc1cccc(-c2ccc(N(c4ccc5c(c4)oc4ccccc45)c4ccc5oc6ccccc6c5c4)cc2)c13 ; c1ccc2cc3c(cc2c1)sc1cc(-c2ccc(N(c4ccc5oc6ccccc6c5c4)c4ccc5oc6ccccc6c5c4)cc2)ccc13 ; c1ccc2cc3c(cc2c1)sc1c(-c2ccc(N(c4ccc5c(c4)oc4ccccc45)c4ccc5c(c4)sc4ccccc45)cc2)cccc13] 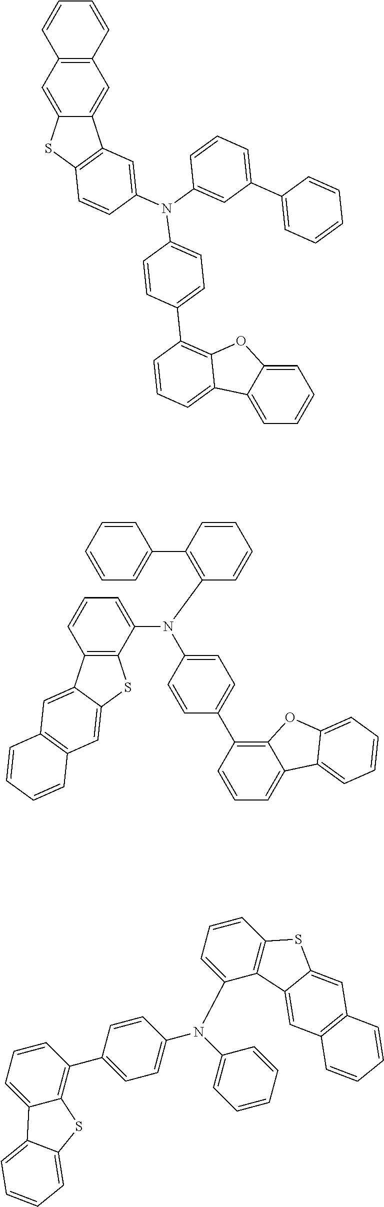 [SMILES: c1ccc(-c2cccc(N(c3ccc(-c4cccc5c4oc4ccccc45)cc3)c3ccc4sc5cc6ccccc6cc5c4c3)c2)cc1 ; c1ccc(-c2ccccc2N(c2ccc(-c3cccc4c3oc3ccccc34)cc2)c2cccc3c2sc2cc4ccccc4cc23)cc1 ; c1ccc(N(c2ccc(-c3cccc4c3sc3ccccc34)cc2)c2cccc3sc4cc5ccccc5cc4c23)cc1] 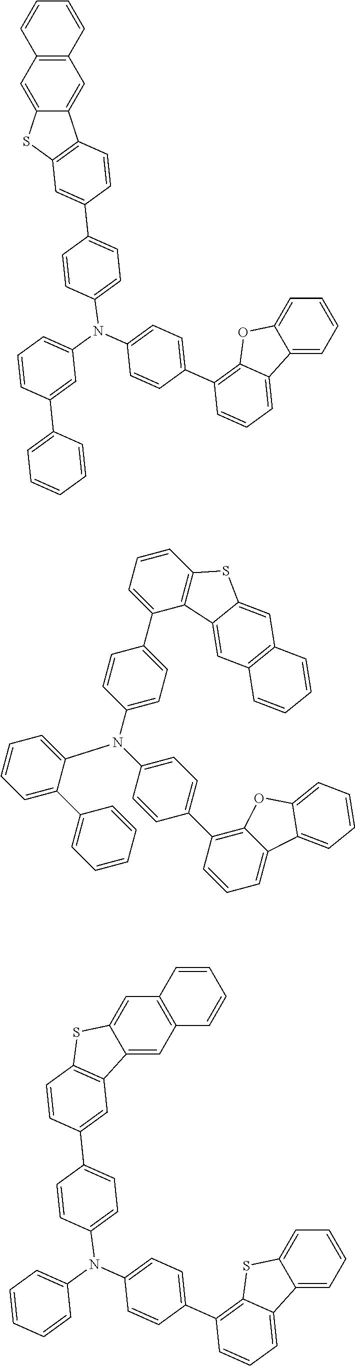 [SMILES: c1ccc(-c2cccc(N(c3ccc(-c4ccc5c(c4)sc4cc6ccccc6cc45)cc3)c3ccc(-c4cccc5c4oc4ccccc45)cc3)c2)cc1 ; c1ccc(-c2ccccc2N(c2ccc(-c3cccc4c3oc3ccccc34)cc2)c2ccc(-c3cccc4sc5cc6ccccc6cc5c34)cc2)cc1 ; c1ccc(N(c2ccc(-c3ccc4sc5cc6ccccc6cc5c4c3)cc2)c2ccc(-c3cccc4c3sc3ccccc34)cc2)cc1] 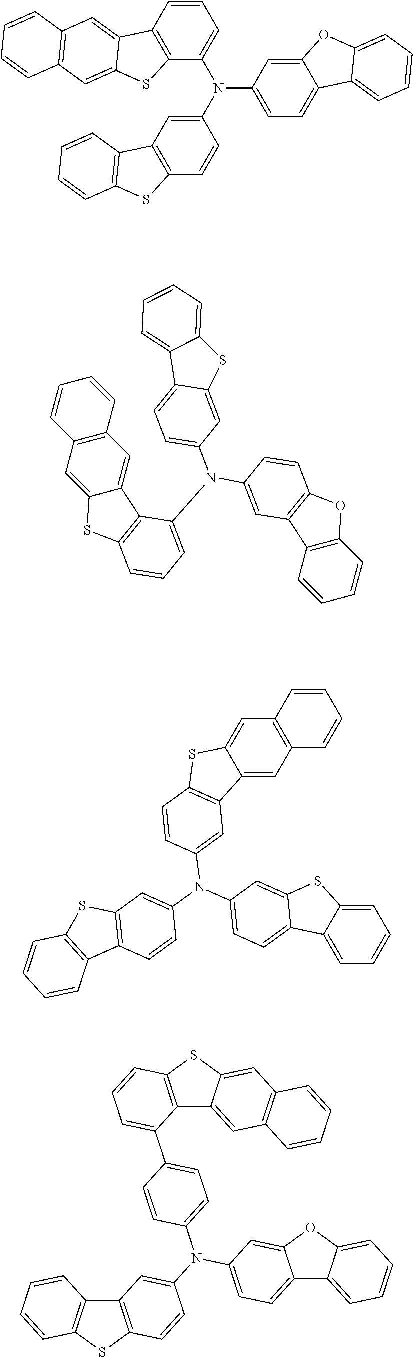 [SMILES: c1ccc2cc3c(cc2c1)sc1c(N(c2ccc4c(c2)oc2ccccc24)c2ccc4sc5ccccc5c4c2)cccc13 ; c1ccc2cc3c(cc2c1)sc1cccc(N(c2ccc4c(c2)sc2ccccc24)c2ccc4oc5ccccc5c4c2)c13 ; c1ccc2cc3c(cc2c1)sc1ccc(N(c2ccc4c(c2)sc2ccccc24)c2ccc4c(c2)sc2ccccc24)cc13 ; c1ccc2cc3c(cc2c1)sc1cccc(-c2ccc(N(c4ccc5c(c4)oc4ccccc45)c4ccc5sc6ccccc6c5c4)cc2)c13] 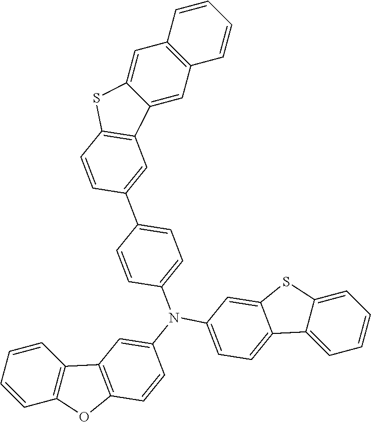 [SMILES: c1ccc2cc3c(cc2c1)sc1ccc(-c2ccc(N(c4ccc5c(c4)sc4ccccc45)c4ccc5oc6ccccc6c5c4)cc2)cc13] 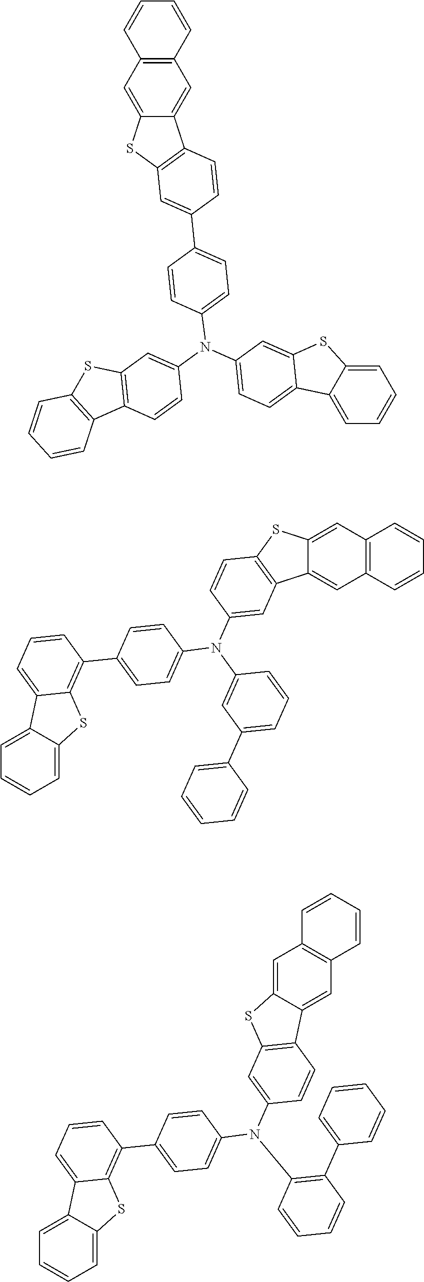 [SMILES: c1ccc2cc3c(cc2c1)sc1cc(-c2ccc(N(c4ccc5c(c4)sc4ccccc45)c4ccc5c(c4)sc4ccccc45)cc2)ccc13 ; c1ccc(-c2cccc(N(c3ccc(-c4cccc5c4sc4ccccc45)cc3)c3ccc4sc5cc6ccccc6cc5c4c3)c2)cc1 ; c1ccc(-c2ccccc2N(c2ccc(-c3cccc4c3sc3ccccc34)cc2)c2ccc3c(c2)sc2cc4ccccc4cc23)cc1] 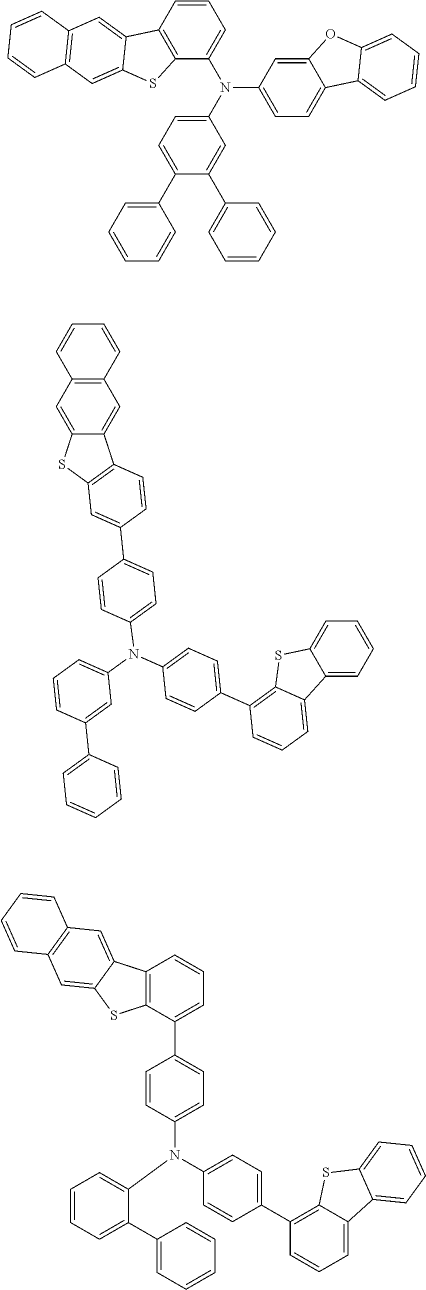 [SMILES: c1ccc(-c2ccc(N(c3ccc4c(c3)oc3ccccc34)c3cccc4c3sc3cc5ccccc5cc34)cc2-c2ccccc2)cc1 ; c1ccc(-c2cccc(N(c3ccc(-c4ccc5c(c4)sc4cc6ccccc6cc45)cc3)c3ccc(-c4cccc5c4sc4ccccc45)cc3)c2)cc1 ; c1ccc(-c2ccccc2N(c2ccc(-c3cccc4c3sc3ccccc34)cc2)c2ccc(-c3cccc4c3sc3cc5ccccc5cc34)cc2)cc1] 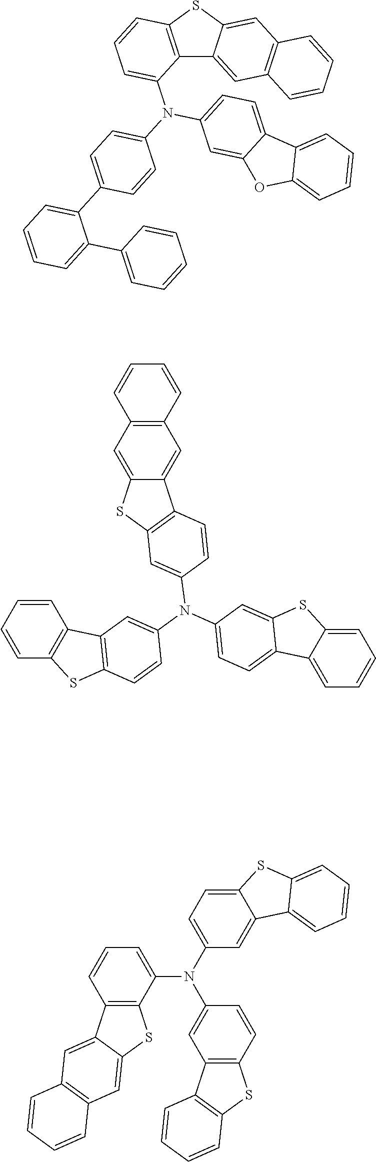 [SMILES: c1ccc(-c2ccccc2-c2ccc(N(c3ccc4c(c3)oc3ccccc34)c3cccc4sc5cc6ccccc6cc5c34)cc2)cc1 ; c1ccc2cc3c(cc2c1)sc1cc(N(c2ccc4c(c2)sc2ccccc24)c2ccc4sc5ccccc5c4c2)ccc13 ; c1ccc2cc3c(cc2c1)sc1c(N(c2ccc4sc5ccccc5c4c2)c2ccc4sc5ccccc5c4c2)cccc13] 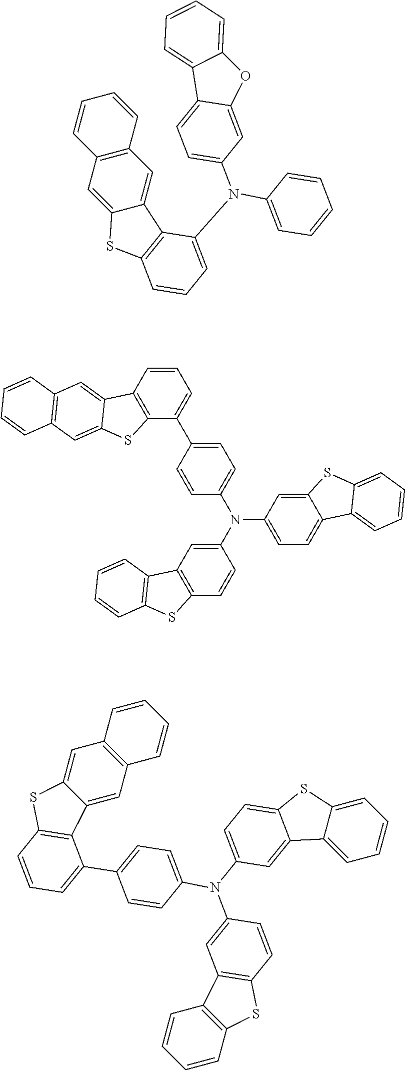 [SMILES: c1ccc(N(c2ccc3c(c2)oc2ccccc23)c2cccc3sc4cc5ccccc5cc4c23)cc1 ; c1ccc2cc3c(cc2c1)sc1c(-c2ccc(N(c4ccc5c(c4)sc4ccccc45)c4ccc5sc6ccccc6c5c4)cc2)cccc13 ; c1ccc2cc3c(cc2c1)sc1cccc(-c2ccc(N(c4ccc5sc6ccccc6c5c4)c4ccc5sc6ccccc6c5c4)cc2)c13] 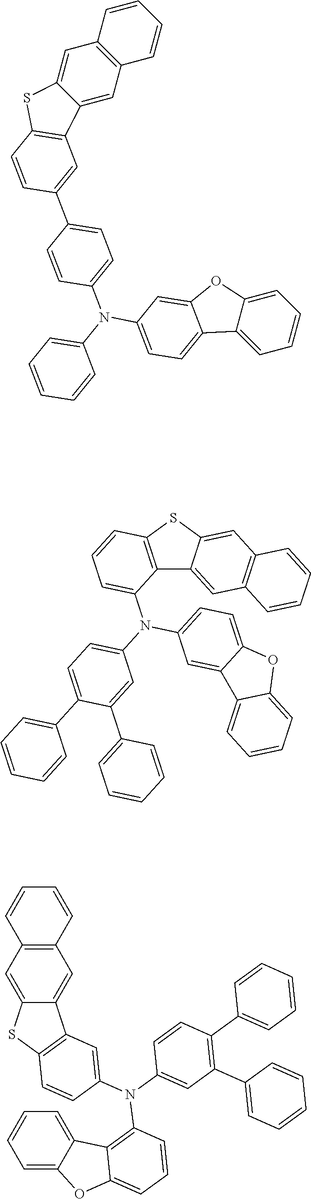 [SMILES: c1ccc(N(c2ccc(-c3ccc4sc5cc6ccccc6cc5c4c3)cc2)c2ccc3c(c2)oc2ccccc23)cc1 ; c1ccc(-c2ccc(N(c3ccc4oc5ccccc5c4c3)c3cccc4sc5cc6ccccc6cc5c34)cc2-c2ccccc2)cc1 ; c1ccc(-c2ccc(N(c3ccc4sc5cc6ccccc6cc5c4c3)c3cccc4oc5ccccc5c34)cc2-c2ccccc2)cc1] 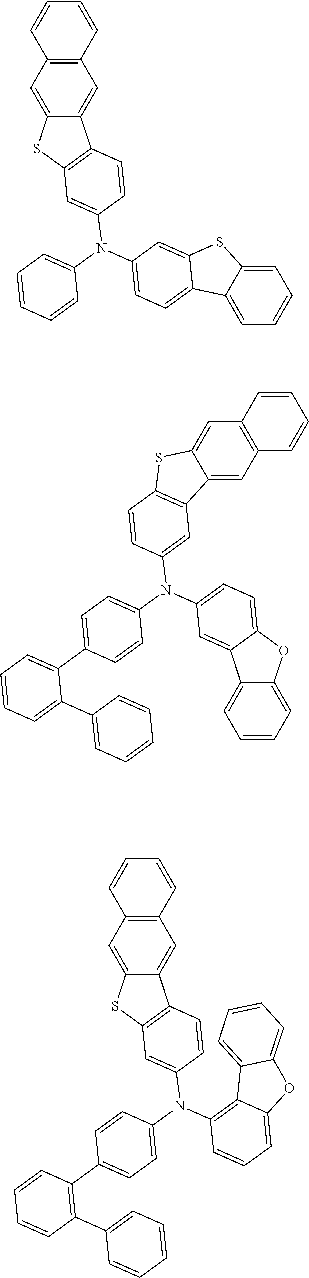 [SMILES: c1ccc(N(c2ccc3c(c2)sc2ccccc23)c2ccc3c(c2)sc2cc4ccccc4cc23)cc1 ; c1ccc(-c2ccccc2-c2ccc(N(c3ccc4oc5ccccc5c4c3)c3ccc4sc5cc6ccccc6cc5c4c3)cc2)cc1 ; c1ccc(-c2ccccc2-c2ccc(N(c3ccc4c(c3)sc3cc5ccccc5cc34)c3cccc4oc5ccccc5c34)cc2)cc1] 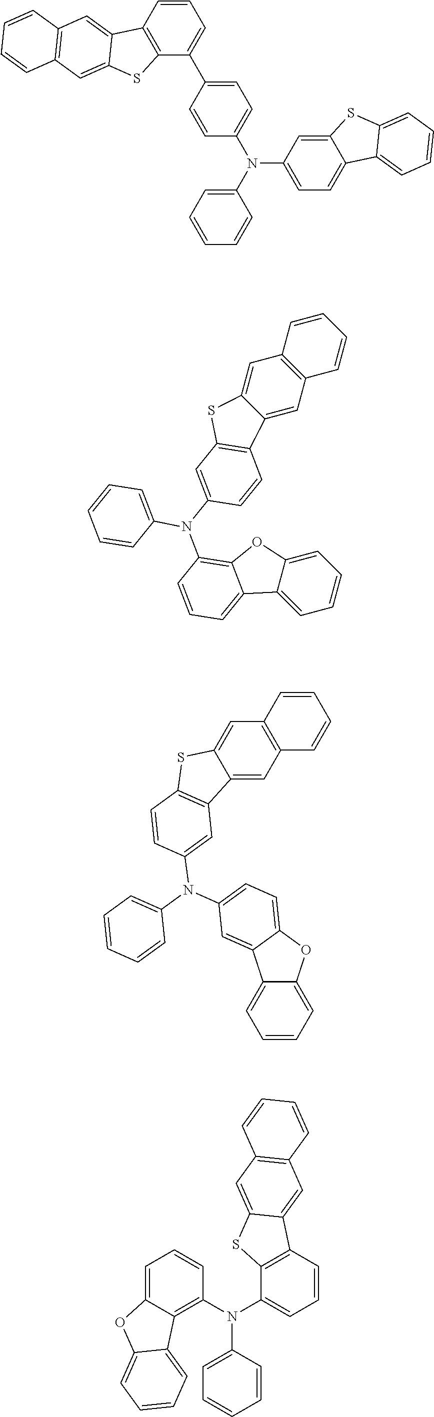 [SMILES: c1ccc(N(c2ccc(-c3cccc4c3sc3cc5ccccc5cc34)cc2)c2ccc3c(c2)sc2ccccc23)cc1 ; c1ccc(N(c2ccc3c(c2)sc2cc4ccccc4cc23)c2cccc3c2oc2ccccc23)cc1 ; c1ccc(N(c2ccc3oc4ccccc4c3c2)c2ccc3sc4cc5ccccc5cc4c3c2)cc1 ; c1ccc(N(c2cccc3c2sc2cc4ccccc4cc23)c2cccc3oc4ccccc4c23)cc1] 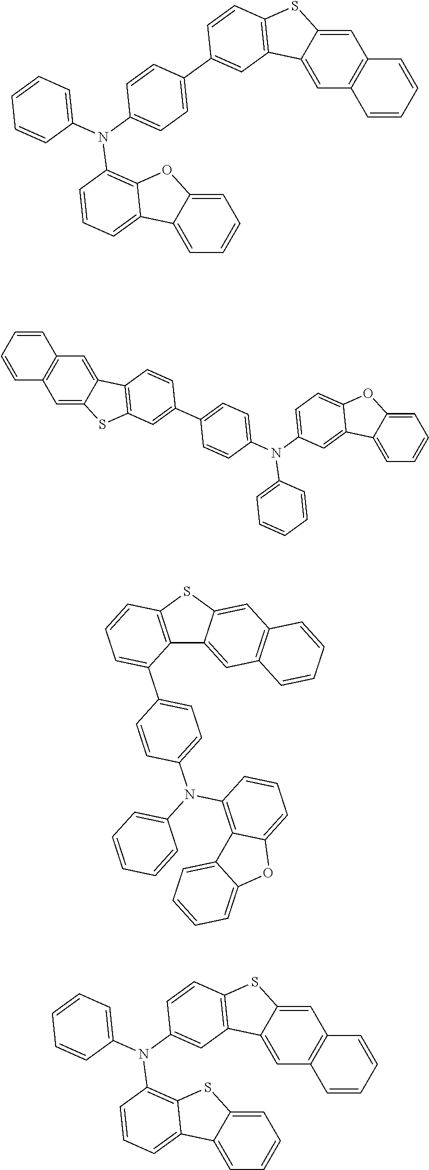 [SMILES: c1ccc(N(c2ccc(-c3ccc4sc5cc6ccccc6cc5c4c3)cc2)c2cccc3c2oc2ccccc23)cc1 ; c1ccc(N(c2ccc(-c3ccc4c(c3)sc3cc5ccccc5cc34)cc2)c2ccc3oc4ccccc4c3c2)cc1 ; c1ccc(N(c2ccc(-c3cccc4sc5cc6ccccc6cc5c34)cc2)c2cccc3oc4ccccc4c23)cc1 ; c1ccc(N(c2ccc3sc4cc5ccccc5cc4c3c2)c2cccc3c2sc2ccccc23)cc1] 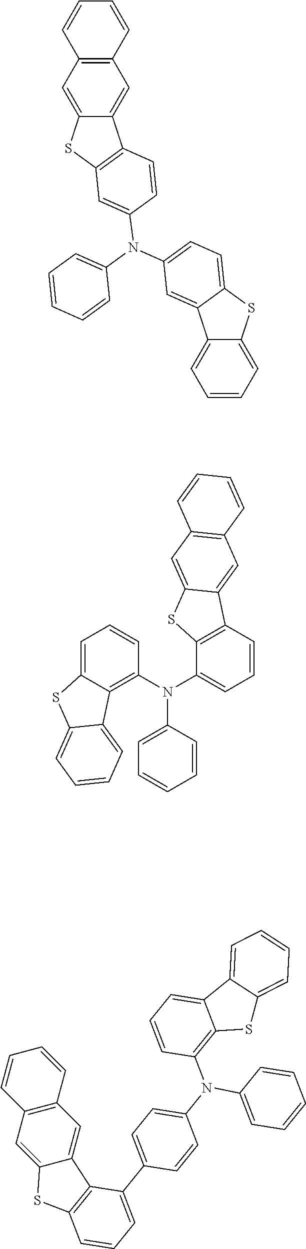 [SMILES: c1ccc(N(c2ccc3c(c2)sc2cc4ccccc4cc23)c2ccc3sc4ccccc4c3c2)cc1 ; c1ccc(N(c2cccc3c2sc2cc4ccccc4cc23)c2cccc3sc4ccccc4c23)cc1 ; c1ccc(N(c2ccc(-c3cccc4sc5cc6ccccc6cc5c34)cc2)c2cccc3c2sc2ccccc23)cc1] 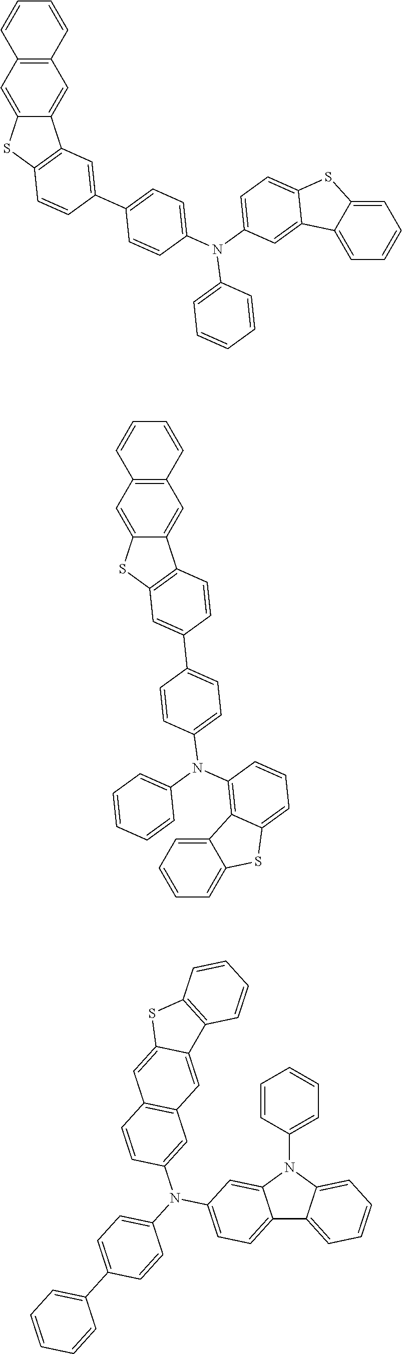 [SMILES: c1ccc(N(c2ccc(-c3ccc4sc5cc6ccccc6cc5c4c3)cc2)c2ccc3sc4ccccc4c3c2)cc1 ; c1ccc(N(c2ccc(-c3ccc4c(c3)sc3cc5ccccc5cc34)cc2)c2cccc3sc4ccccc4c23)cc1 ; c1ccc(-c2ccc(N(c3ccc4cc5sc6ccccc6c5cc4c3)c3ccc4c5ccccc5n(-c5ccccc5)c4c3)cc2)cc1] 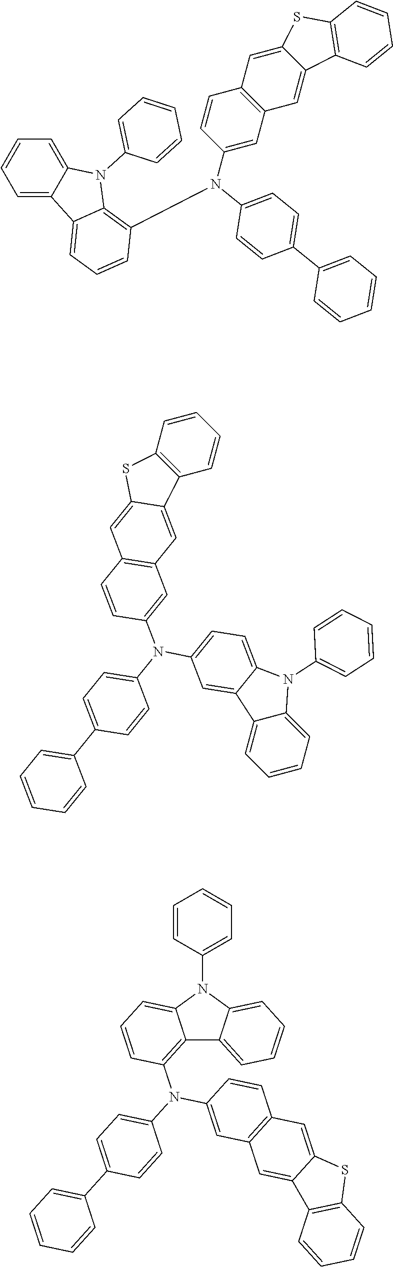 [SMILES: c1ccc(-c2ccc(N(c3ccc4cc5sc6ccccc6c5cc4c3)c3cccc4c5ccccc5n(-c5ccccc5)c34)cc2)cc1 ; c1ccc(-c2ccc(N(c3ccc4cc5sc6ccccc6c5cc4c3)c3ccc4c(c3)c3ccccc3n4-c3ccccc3)cc2)cc1 ; c1ccc(-c2ccc(N(c3ccc4cc5sc6ccccc6c5cc4c3)c3cccc4c3c3ccccc3n4-c3ccccc3)cc2)cc1] 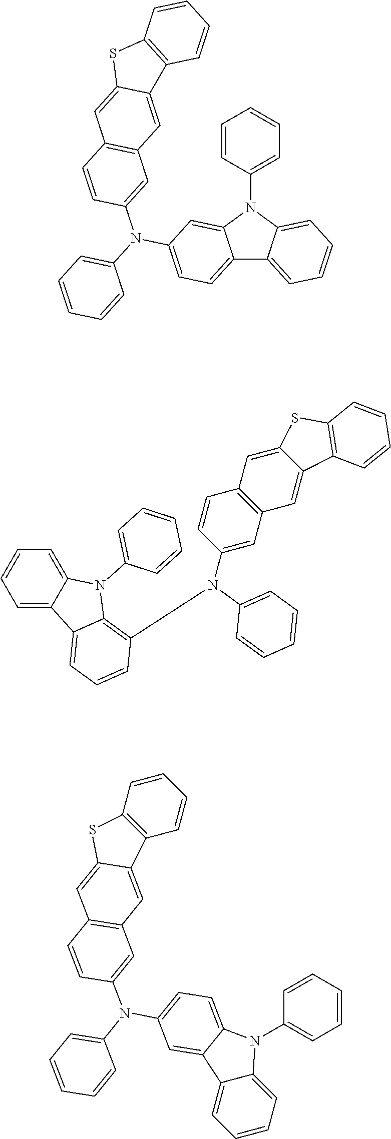 [SMILES: c1ccc(N(c2ccc3cc4sc5ccccc5c4cc3c2)c2ccc3c4ccccc4n(-c4ccccc4)c3c2)cc1 ; c1ccc(N(c2ccc3cc4sc5ccccc5c4cc3c2)c2cccc3c4ccccc4n(-c4ccccc4)c23)cc1 ; c1ccc(N(c2ccc3cc4sc5ccccc5c4cc3c2)c2ccc3c(c2)c2ccccc2n3-c2ccccc2)cc1] 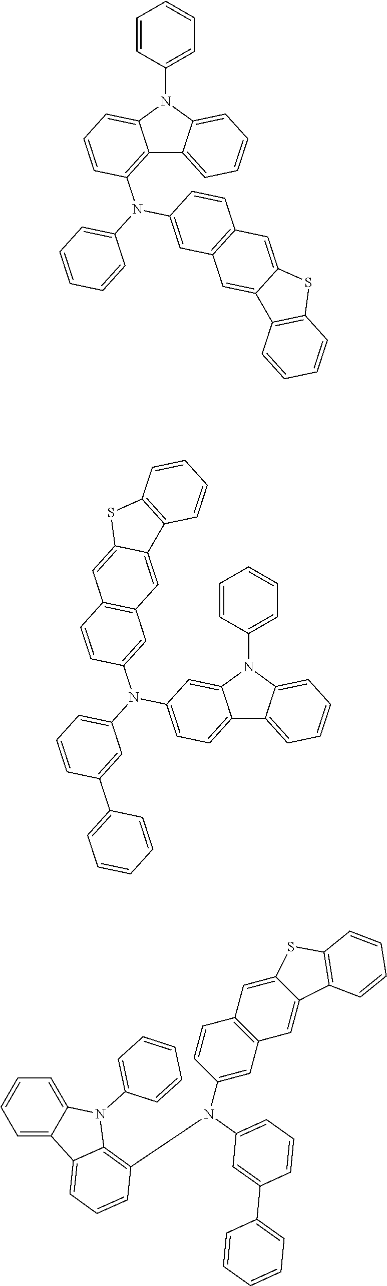 [SMILES: c1ccc(N(c2ccc3cc4sc5ccccc5c4cc3c2)c2cccc3c2c2ccccc2n3-c2ccccc2)cc1 ; c1ccc(-c2cccc(N(c3ccc4cc5sc6ccccc6c5cc4c3)c3ccc4c5ccccc5n(-c5ccccc5)c4c3)c2)cc1 ; c1ccc(-c2cccc(N(c3ccc4cc5sc6ccccc6c5cc4c3)c3cccc4c5ccccc5n(-c5ccccc5)c34)c2)cc1] 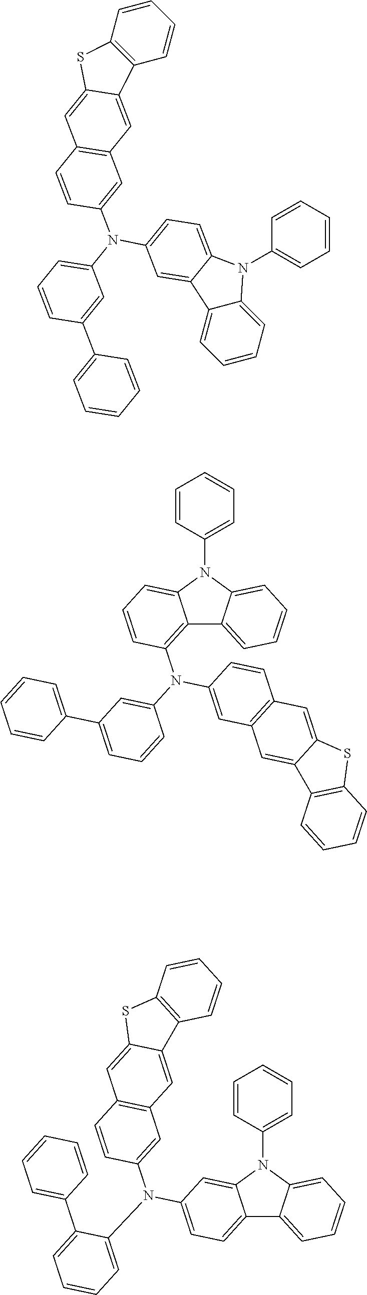 [SMILES: c1ccc(-c2cccc(N(c3ccc4cc5sc6ccccc6c5cc4c3)c3ccc4c(c3)c3ccccc3n4-c3ccccc3)c2)cc1 ; c1ccc(-c2cccc(N(c3ccc4cc5sc6ccccc6c5cc4c3)c3cccc4c3c3ccccc3n4-c3ccccc3)c2)cc1 ; c1ccc(-c2ccccc2N(c2ccc3cc4sc5ccccc5c4cc3c2)c2ccc3c4ccccc4n(-c4ccccc4)c3c2)cc1] 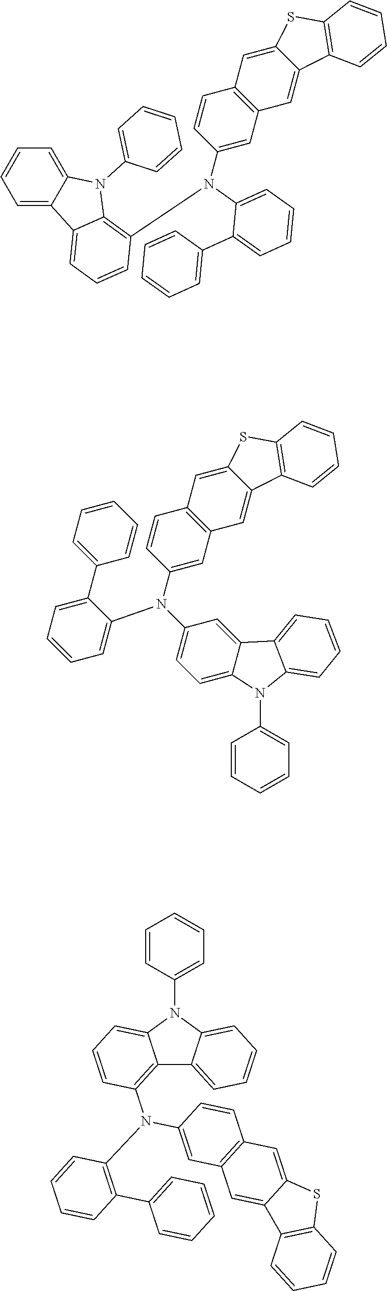 [SMILES: c1ccc(-c2ccccc2N(c2ccc3cc4sc5ccccc5c4cc3c2)c2cccc3c4ccccc4n(-c4ccccc4)c23)cc1 ; c1ccc(-c2ccccc2N(c2ccc3cc4sc5ccccc5c4cc3c2)c2ccc3c(c2)c2ccccc2n3-c2ccccc2)cc1 ; c1ccc(-c2ccccc2N(c2ccc3cc4sc5ccccc5c4cc3c2)c2cccc3c2c2ccccc2n3-c2ccccc2)cc1] 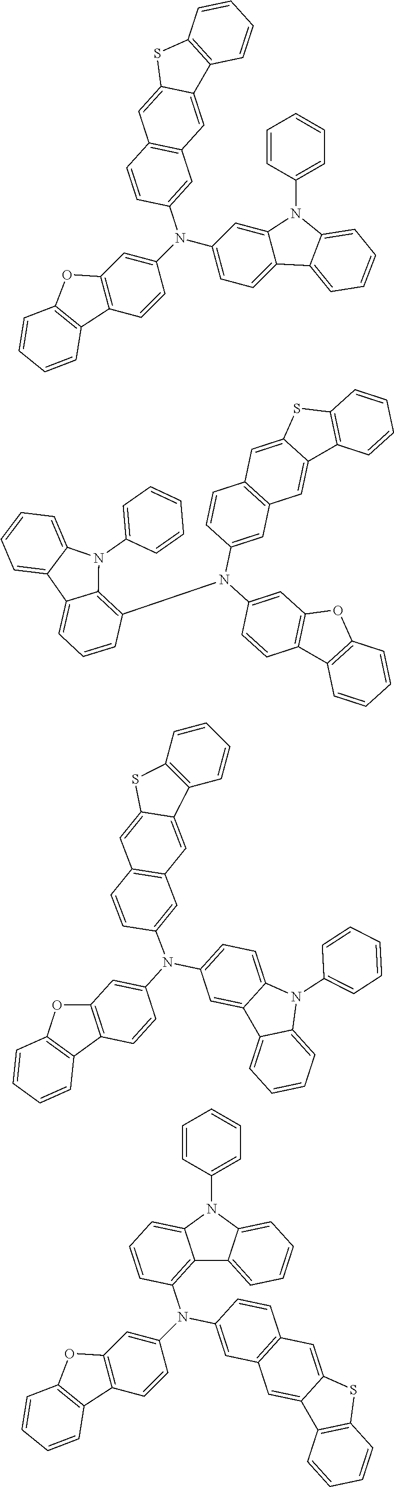 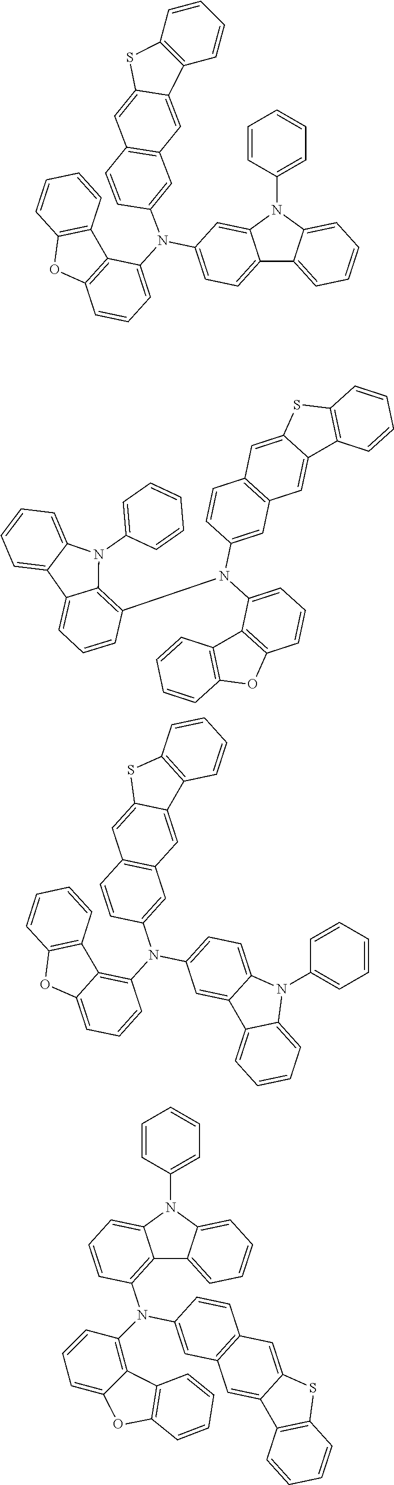 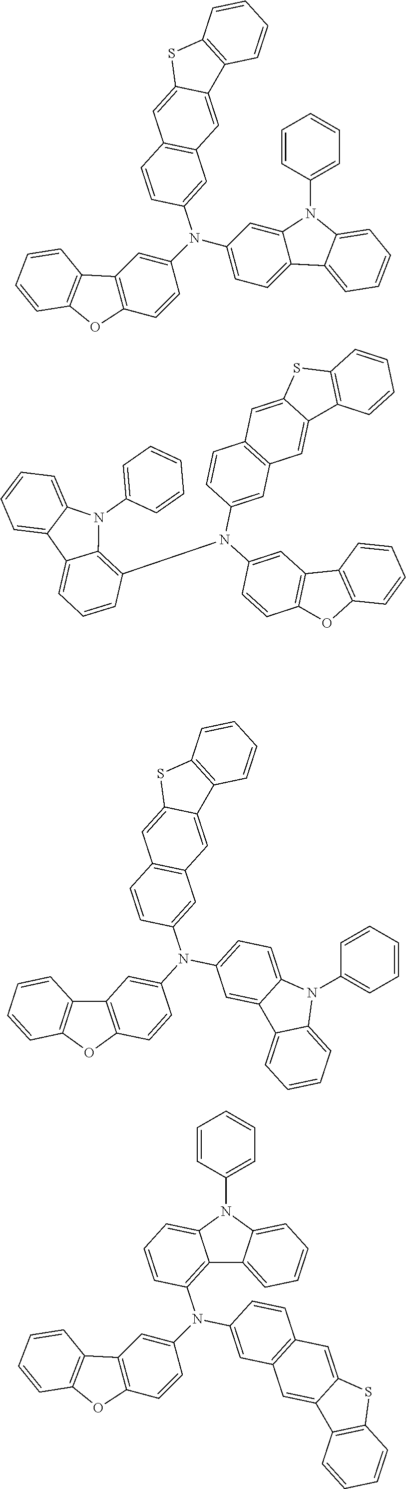 [SMILES: c1ccc(-n2c3ccccc3c3ccc(N(c4ccc5cc6sc7ccccc7c6cc5c4)c4ccc5oc6ccccc6c5c4)cc32)cc1 ; c1ccc(-n2c3ccccc3c3cccc(N(c4ccc5cc6sc7ccccc7c6cc5c4)c4ccc5oc6ccccc6c5c4)c32)cc1 ; c1ccc(-n2c3ccccc3c3cc(N(c4ccc5cc6sc7ccccc7c6cc5c4)c4ccc5oc6ccccc6c5c4)ccc32)cc1 ; c1ccc(-n2c3ccccc3c3c(N(c4ccc5cc6sc7ccccc7c6cc5c4)c4ccc5oc6ccccc6c5c4)cccc32)cc1] 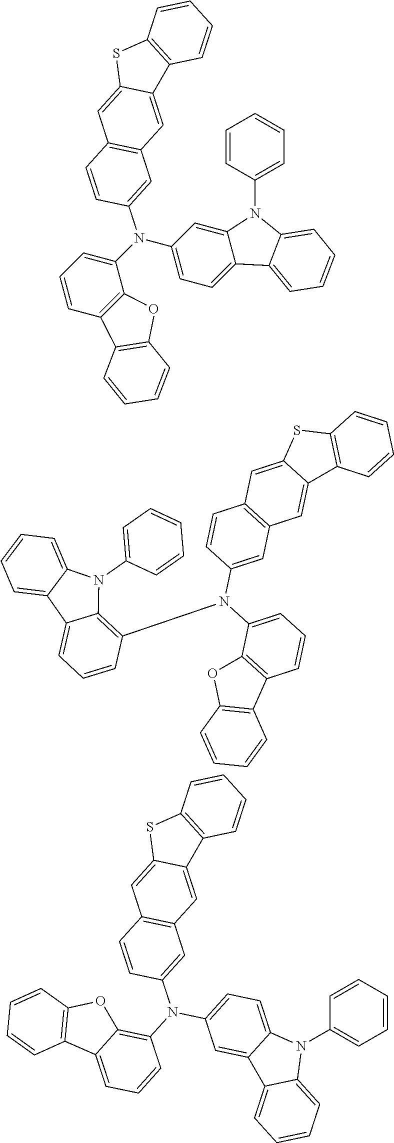 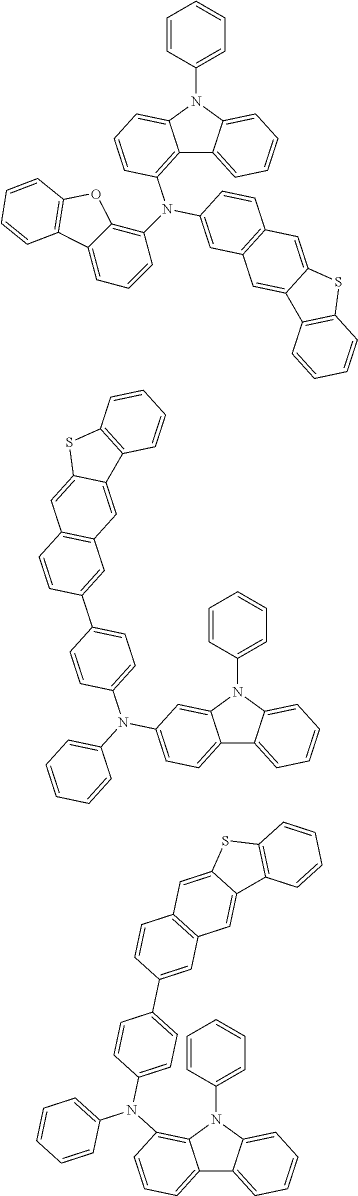 [SMILES: c1ccc(-n2c3ccccc3c3c(N(c4ccc5cc6sc7ccccc7c6cc5c4)c4cccc5c4oc4ccccc45)cccc32)cc1 ; c1ccc(N(c2ccc(-c3ccc4cc5sc6ccccc6c5cc4c3)cc2)c2ccc3c4ccccc4n(-c4ccccc4)c3c2)cc1 ; c1ccc(N(c2ccc(-c3ccc4cc5sc6ccccc6c5cc4c3)cc2)c2cccc3c4ccccc4n(-c4ccccc4)c23)cc1] 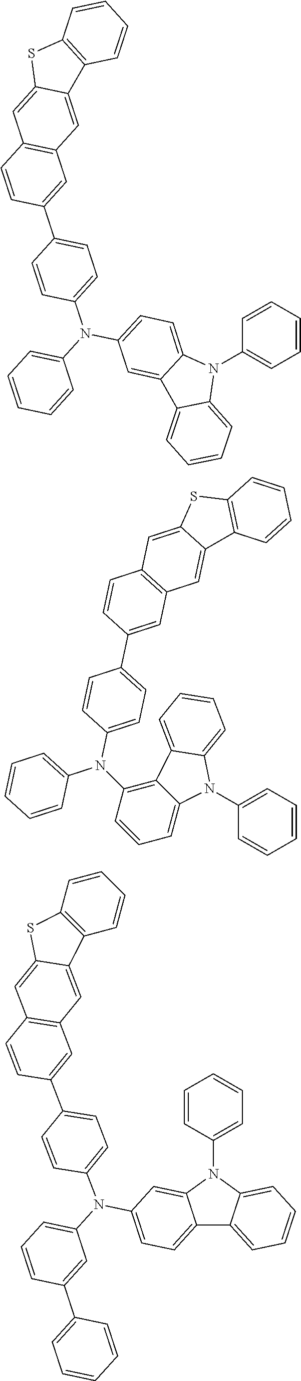 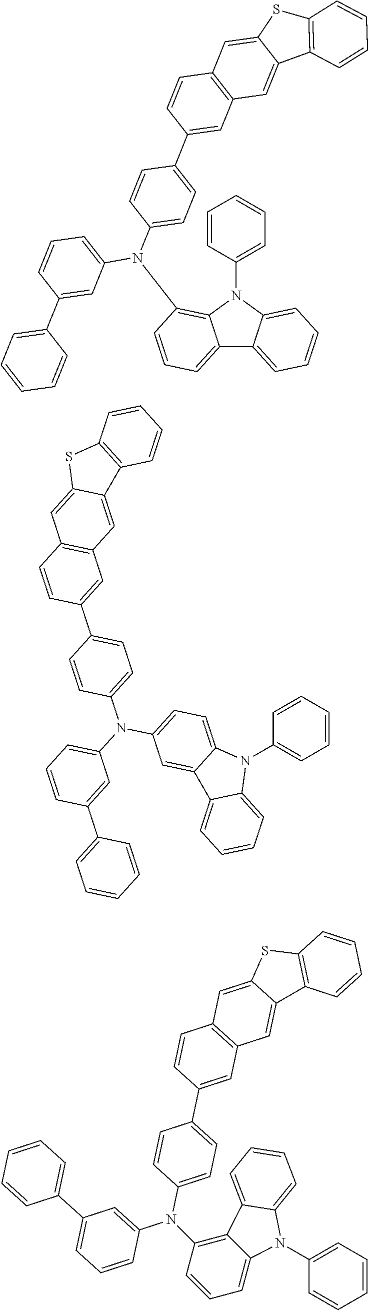 [SMILES: c1ccc(-c2cccc(N(c3ccc(-c4ccc5cc6sc7ccccc7c6cc5c4)cc3)c3cccc4c5ccccc5n(-c5ccccc5)c34)c2)cc1 ; c1ccc(-c2cccc(N(c3ccc(-c4ccc5cc6sc7ccccc7c6cc5c4)cc3)c3ccc4c(c3)c3ccccc3n4-c3ccccc3)c2)cc1 ; c1ccc(-c2cccc(N(c3ccc(-c4ccc5cc6sc7ccccc7c6cc5c4)cc3)c3cccc4c3c3ccccc3n4-c3ccccc3)c2)cc1] 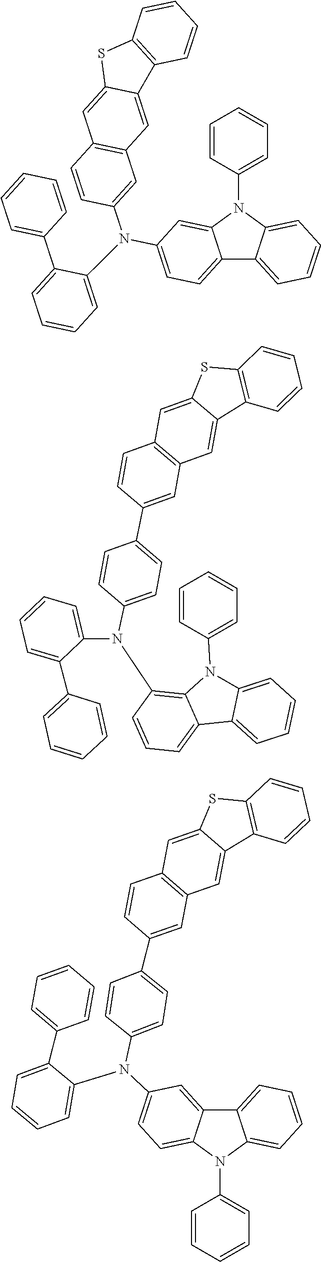 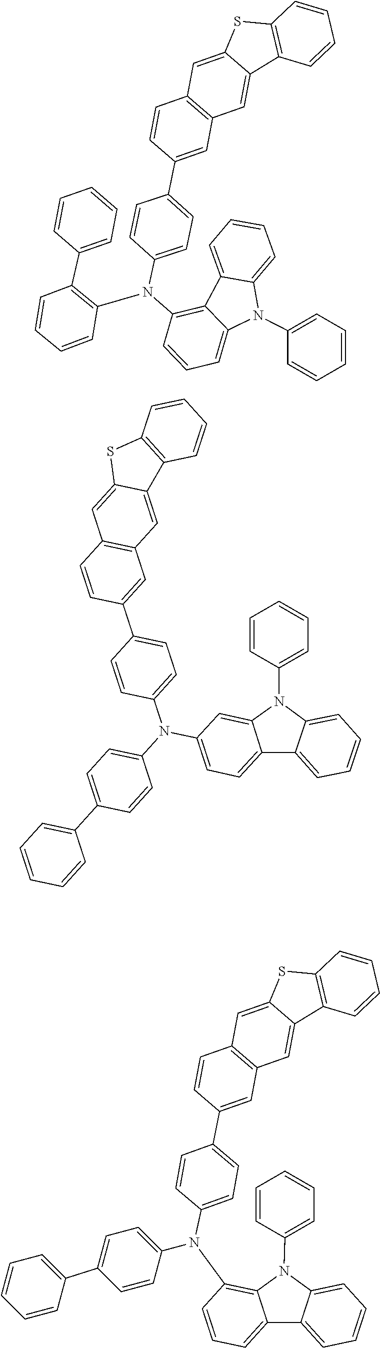 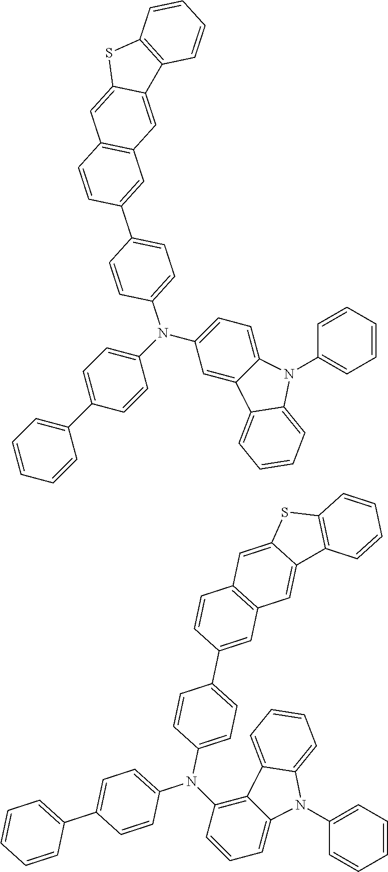 [SMILES: c1ccc(-c2ccc(N(c3ccc(-c4ccc5cc6sc7ccccc7c6cc5c4)cc3)c3ccc4c(c3)c3ccccc3n4-c3ccccc3)cc2)cc1 ; c1ccc(-c2ccc(N(c3ccc(-c4ccc5cc6sc7ccccc7c6cc5c4)cc3)c3cccc4c3c3ccccc3n4-c3ccccc3)cc2)cc1] 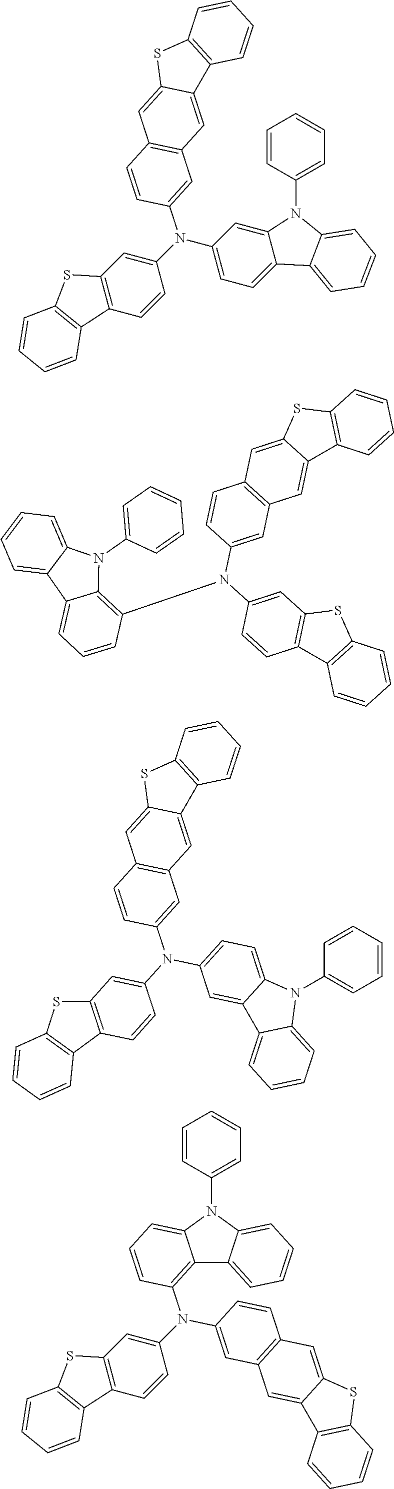 [SMILES: c1ccc(-n2c3ccccc3c3ccc(N(c4ccc5cc6sc7ccccc7c6cc5c4)c4ccc5c(c4)sc4ccccc45)cc32)cc1 ; c1ccc(-n2c3ccccc3c3cccc(N(c4ccc5cc6sc7ccccc7c6cc5c4)c4ccc5c(c4)sc4ccccc45)c32)cc1 ; c1ccc(-n2c3ccccc3c3cc(N(c4ccc5cc6sc7ccccc7c6cc5c4)c4ccc5c(c4)sc4ccccc45)ccc32)cc1 ; c1ccc(-n2c3ccccc3c3c(N(c4ccc5cc6sc7ccccc7c6cc5c4)c4ccc5c(c4)sc4ccccc45)cccc32)cc1] 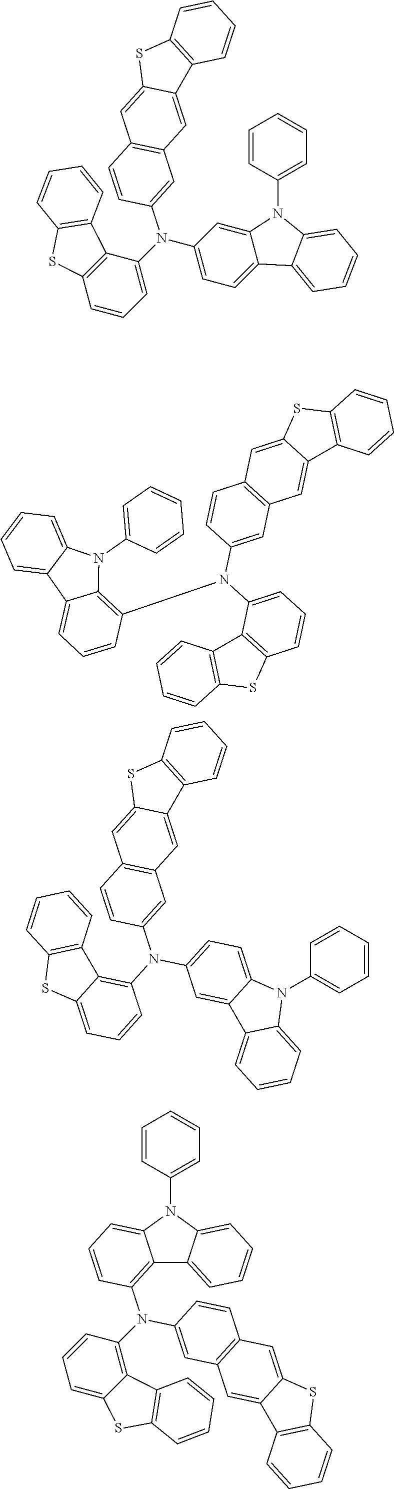 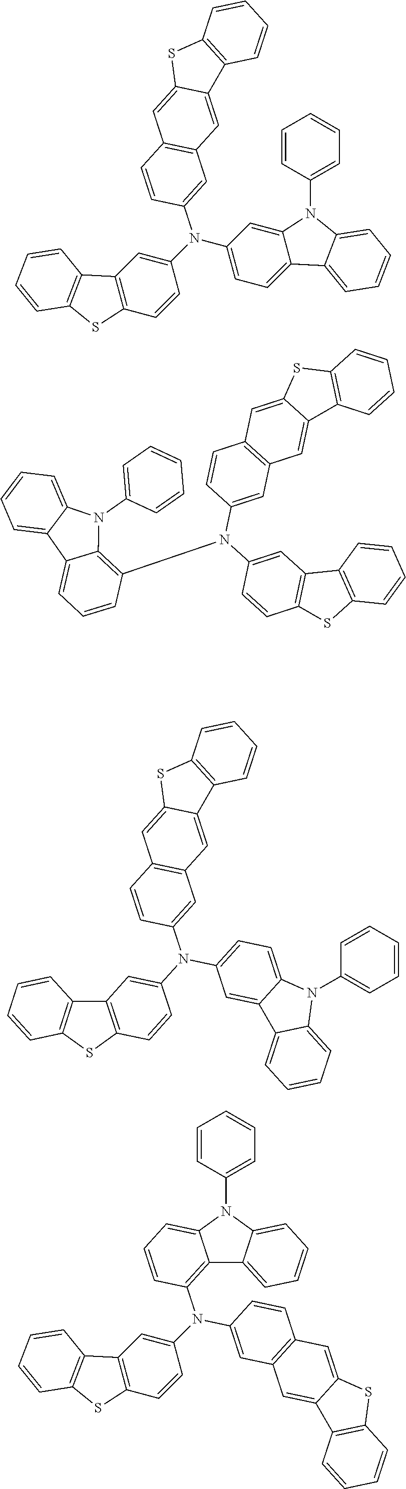 [SMILES: c1ccc(-n2c3ccccc3c3ccc(N(c4ccc5cc6sc7ccccc7c6cc5c4)c4ccc5sc6ccccc6c5c4)cc32)cc1 ; c1ccc(-n2c3ccccc3c3cccc(N(c4ccc5cc6sc7ccccc7c6cc5c4)c4ccc5sc6ccccc6c5c4)c32)cc1 ; c1ccc(-n2c3ccccc3c3cc(N(c4ccc5cc6sc7ccccc7c6cc5c4)c4ccc5sc6ccccc6c5c4)ccc32)cc1 ; c1ccc(-n2c3ccccc3c3c(N(c4ccc5cc6sc7ccccc7c6cc5c4)c4ccc5sc6ccccc6c5c4)cccc32)cc1] 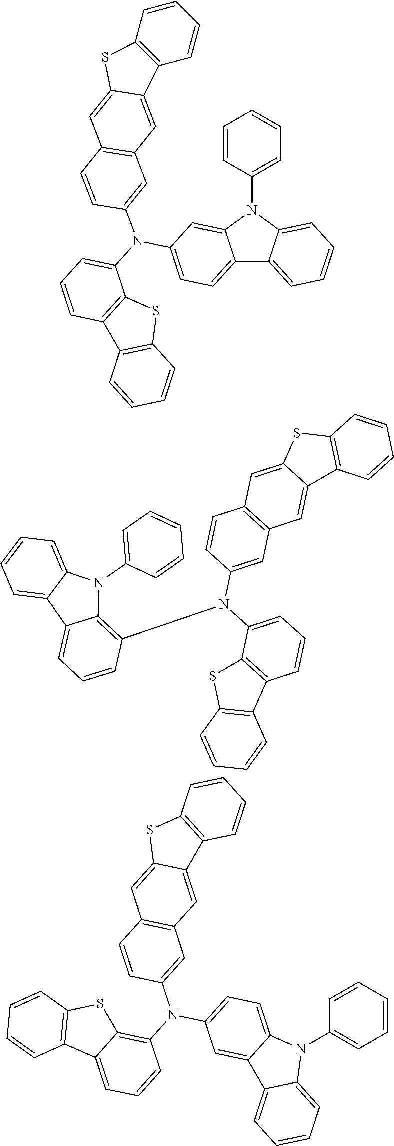 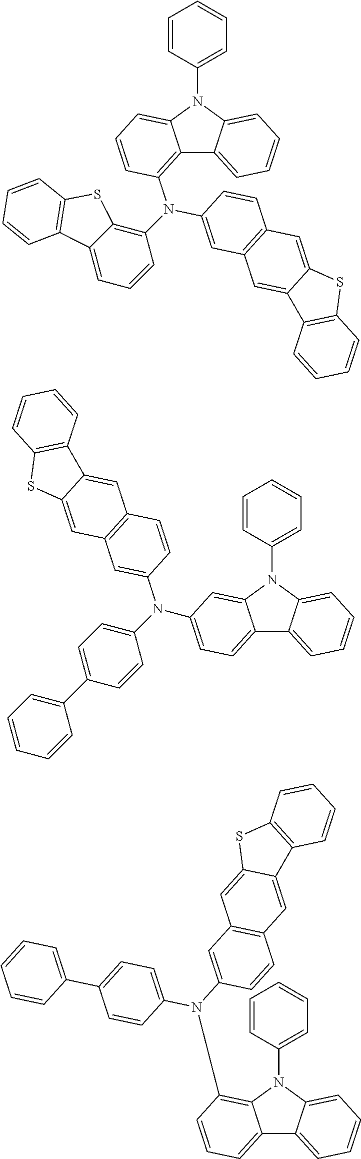 [SMILES: c1ccc(-n2c3ccccc3c3c(N(c4ccc5cc6sc7ccccc7c6cc5c4)c4cccc5c4sc4ccccc45)cccc32)cc1 ; c1ccc(-c2ccc(N(c3ccc4cc5c(cc4c3)sc3ccccc35)c3ccc4c5ccccc5n(-c5ccccc5)c4c3)cc2)cc1 ; c1ccc(-c2ccc(N(c3ccc4cc5c(cc4c3)sc3ccccc35)c3cccc4c5ccccc5n(-c5ccccc5)c34)cc2)cc1] 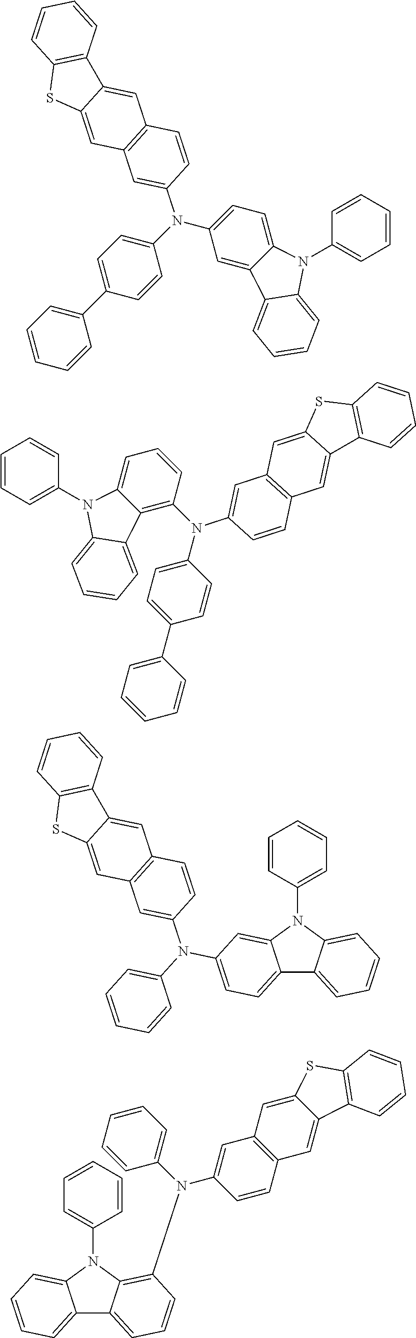 [SMILES: c1ccc(-c2ccc(N(c3ccc4cc5c(cc4c3)sc3ccccc35)c3ccc4c(c3)c3ccccc3n4-c3ccccc3)cc2)cc1 ; c1ccc(-c2ccc(N(c3ccc4cc5c(cc4c3)sc3ccccc35)c3cccc4c3c3ccccc3n4-c3ccccc3)cc2)cc1 ; c1ccc(N(c2ccc3cc4c(cc3c2)sc2ccccc24)c2ccc3c4ccccc4n(-c4ccccc4)c3c2)cc1 ; c1ccc(N(c2ccc3cc4c(cc3c2)sc2ccccc24)c2cccc3c4ccccc4n(-c4ccccc4)c23)cc1] 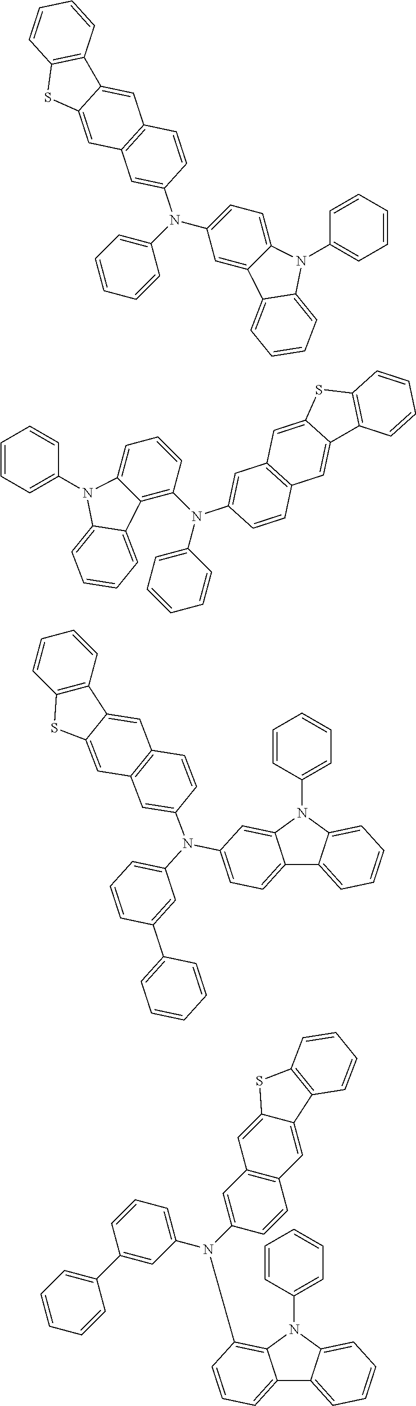 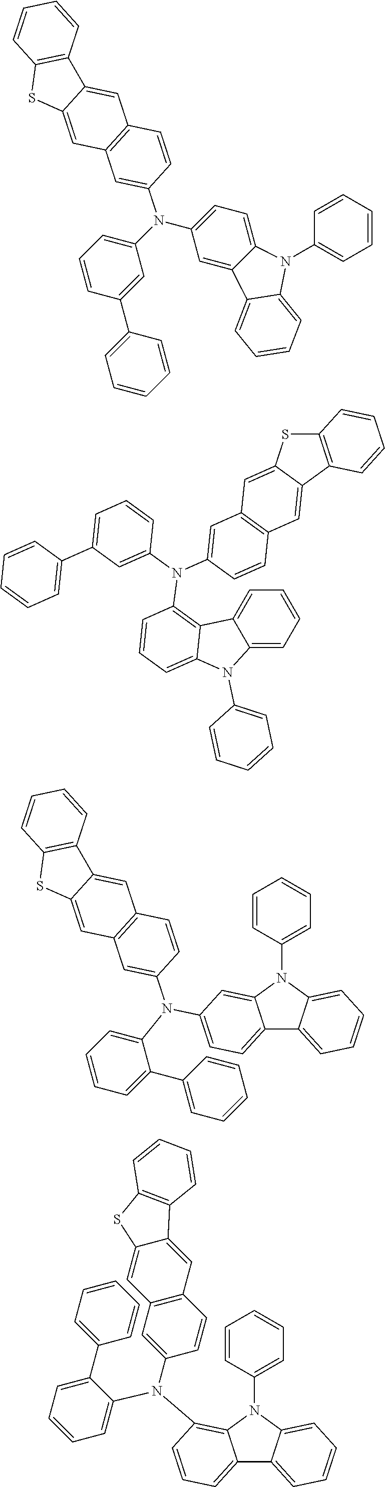 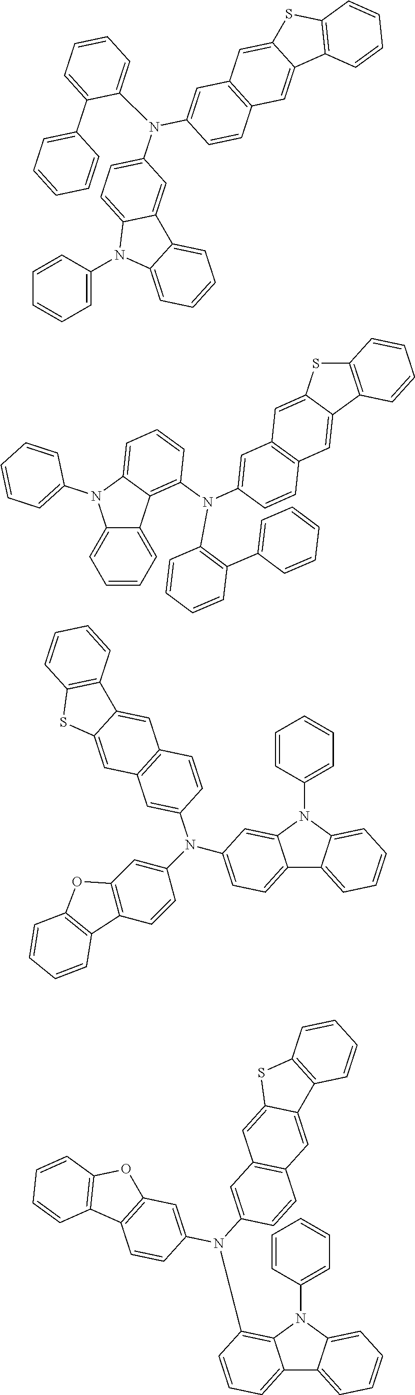 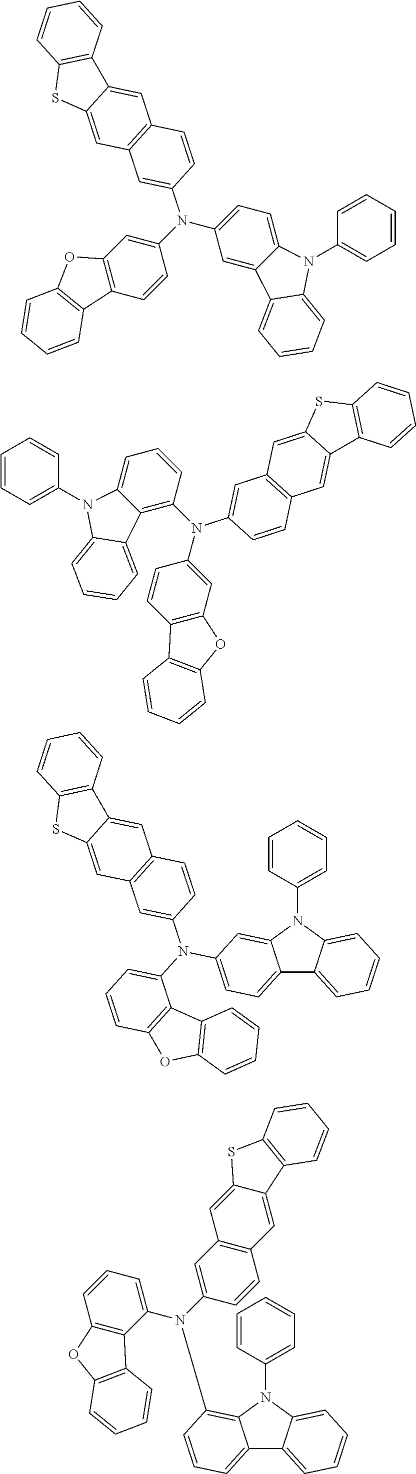 [SMILES: c1ccc(-n2c3ccccc3c3cc(N(c4ccc5cc6c(cc5c4)sc4ccccc46)c4ccc5c(c4)oc4ccccc45)ccc32)cc1 ; c1ccc(-n2c3ccccc3c3c(N(c4ccc5cc6c(cc5c4)sc4ccccc46)c4ccc5c(c4)oc4ccccc45)cccc32)cc1 ; c1ccc(-n2c3ccccc3c3ccc(N(c4ccc5cc6c(cc5c4)sc4ccccc46)c4cccc5oc6ccccc6c45)cc32)cc1 ; c1ccc(-n2c3ccccc3c3cccc(N(c4ccc5cc6c(cc5c4)sc4ccccc46)c4cccc5oc6ccccc6c45)c32)cc1] 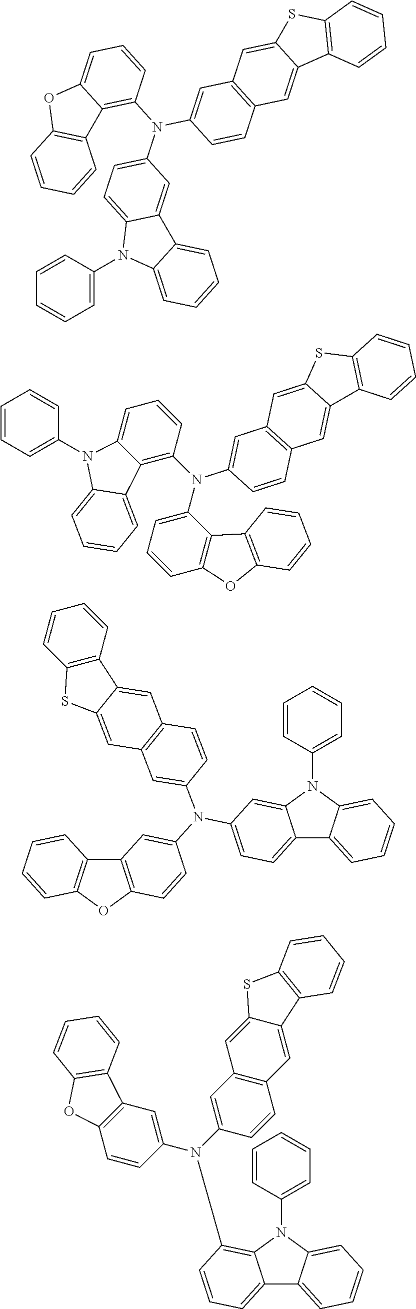 [SMILES: c1ccc(-n2c3ccccc3c3cc(N(c4ccc5cc6c(cc5c4)sc4ccccc46)c4cccc5oc6ccccc6c45)ccc32)cc1 ; c1ccc(-n2c3ccccc3c3c(N(c4ccc5cc6c(cc5c4)sc4ccccc46)c4cccc5oc6ccccc6c45)cccc32)cc1 ; c1ccc(-n2c3ccccc3c3ccc(N(c4ccc5cc6c(cc5c4)sc4ccccc46)c4ccc5oc6ccccc6c5c4)cc32)cc1 ; c1ccc(-n2c3ccccc3c3cccc(N(c4ccc5cc6c(cc5c4)sc4ccccc46)c4ccc5oc6ccccc6c5c4)c32)cc1] 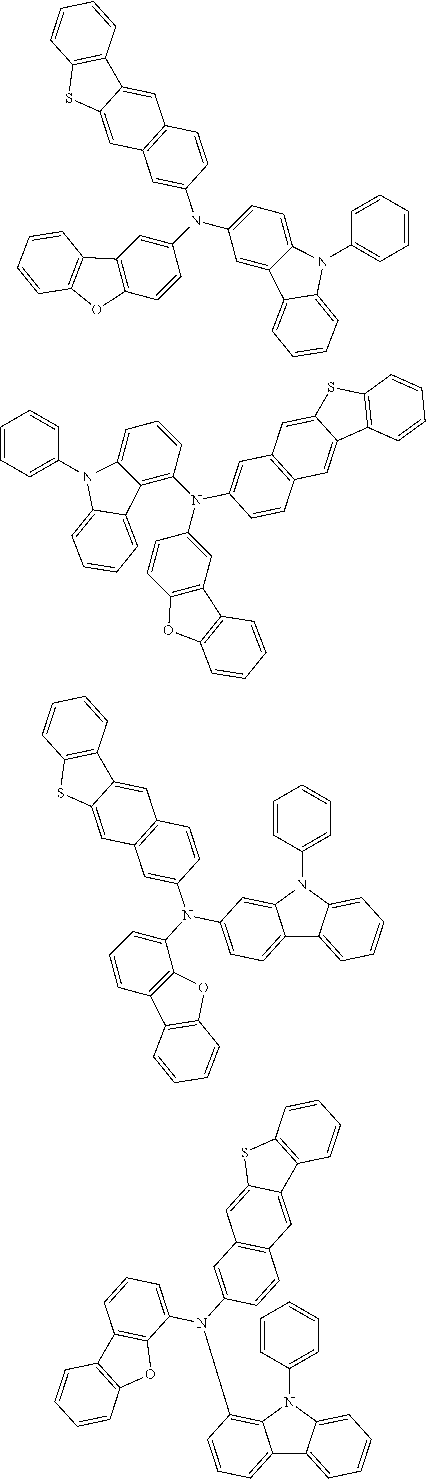 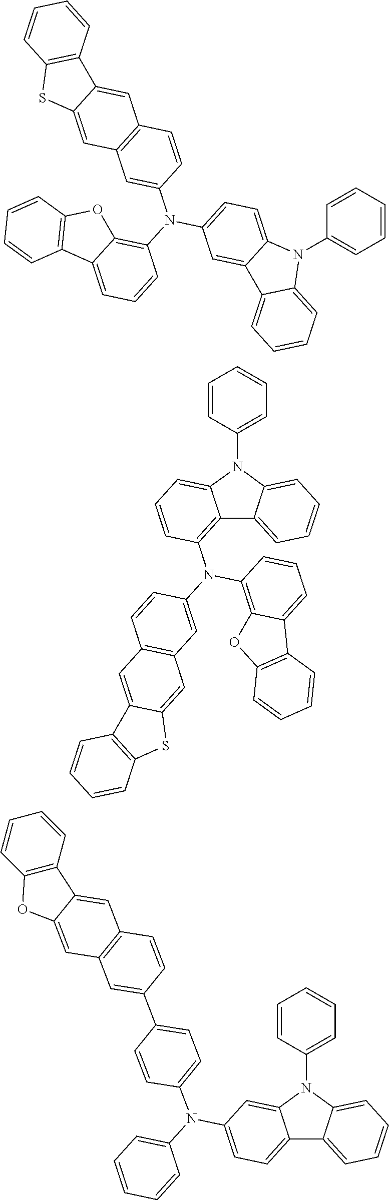 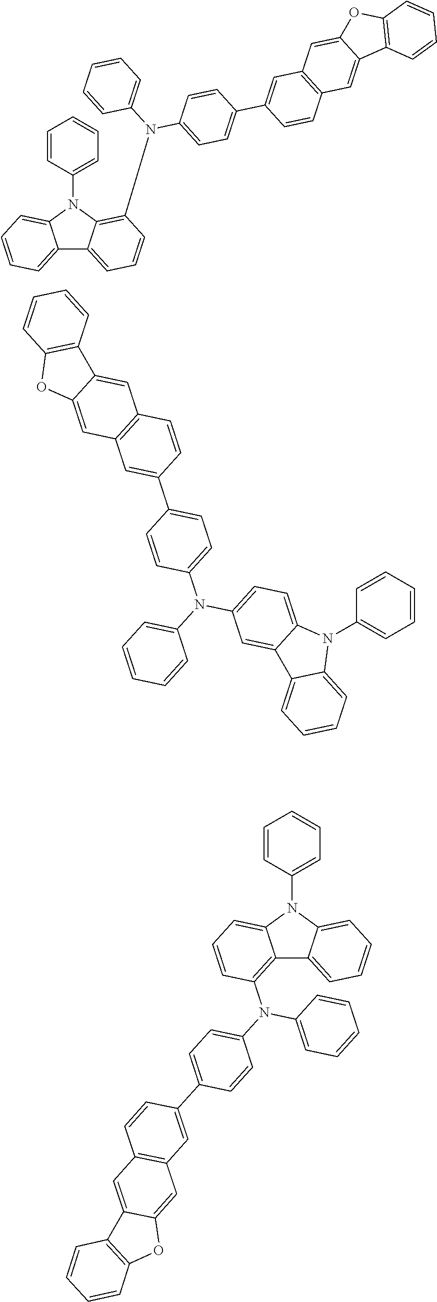 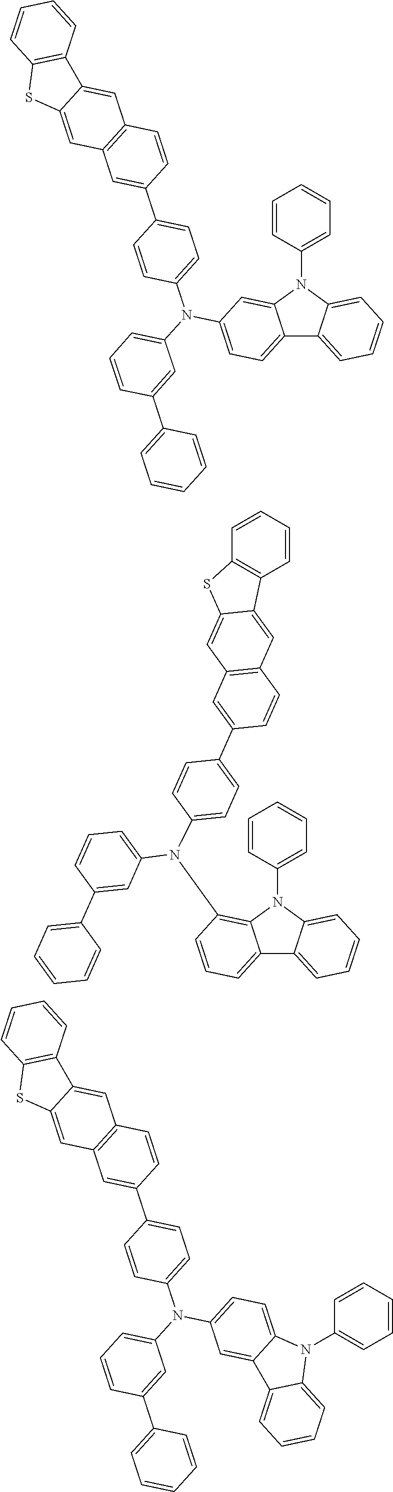 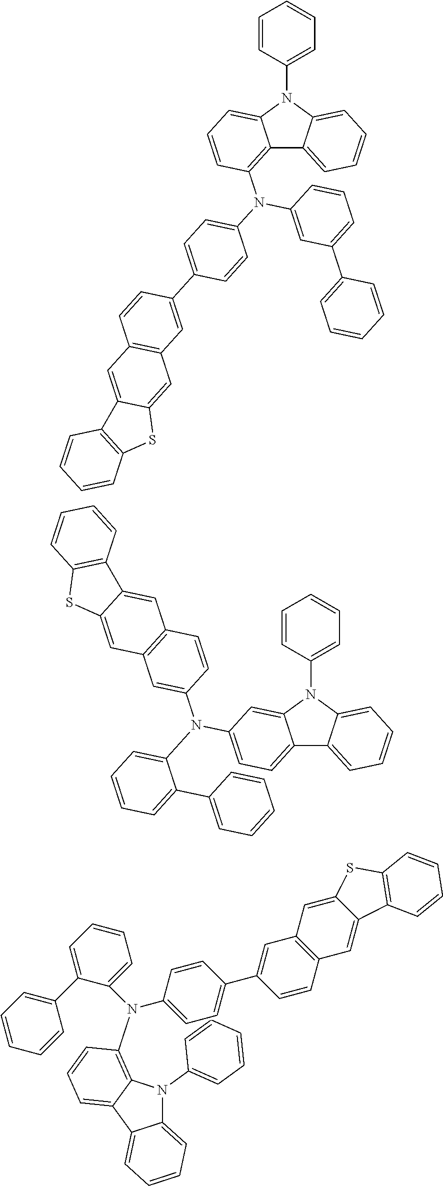 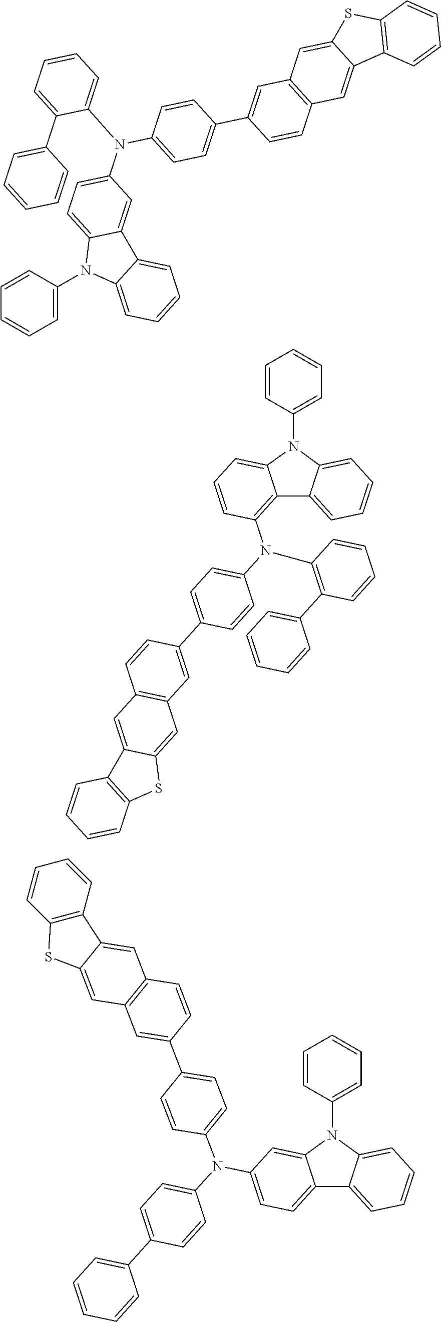 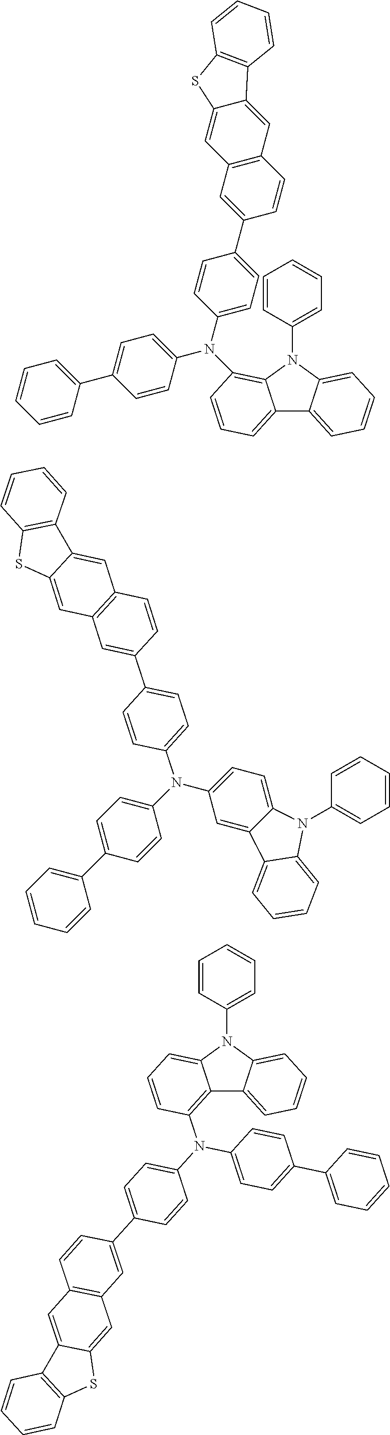 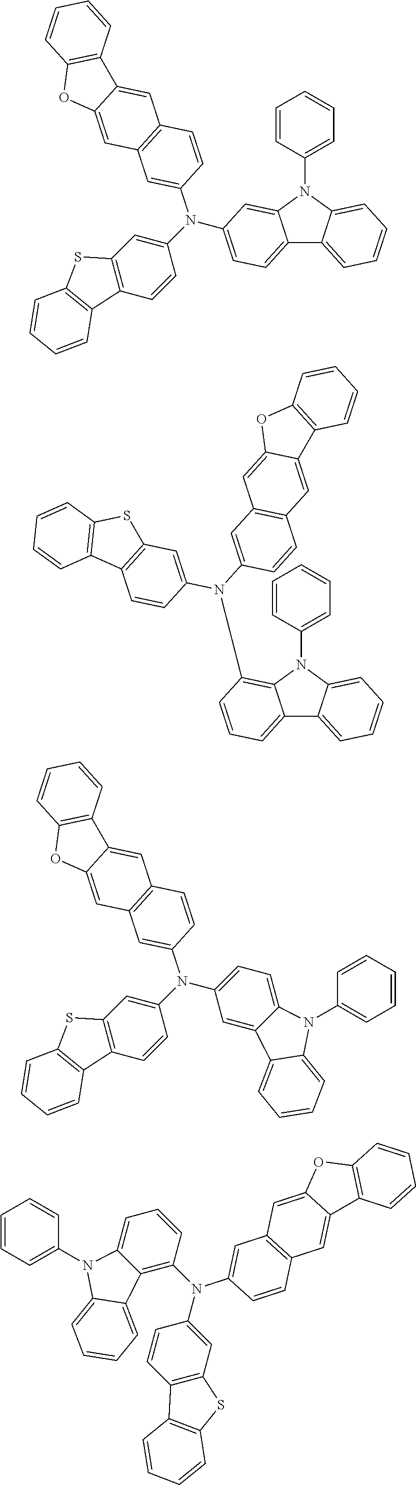 [SMILES: c1ccc(-n2c3ccccc3c3ccc(N(c4ccc5cc6c(cc5c4)oc4ccccc46)c4ccc5c(c4)sc4ccccc45)cc32)cc1 ; c1ccc(-n2c3ccccc3c3cccc(N(c4ccc5cc6c(cc5c4)oc4ccccc46)c4ccc5c(c4)sc4ccccc45)c32)cc1 ; c1ccc(-n2c3ccccc3c3cc(N(c4ccc5cc6c(cc5c4)oc4ccccc46)c4ccc5c(c4)sc4ccccc45)ccc32)cc1 ; c1ccc(-n2c3ccccc3c3c(N(c4ccc5cc6c(cc5c4)oc4ccccc46)c4ccc5c(c4)sc4ccccc45)cccc32)cc1] 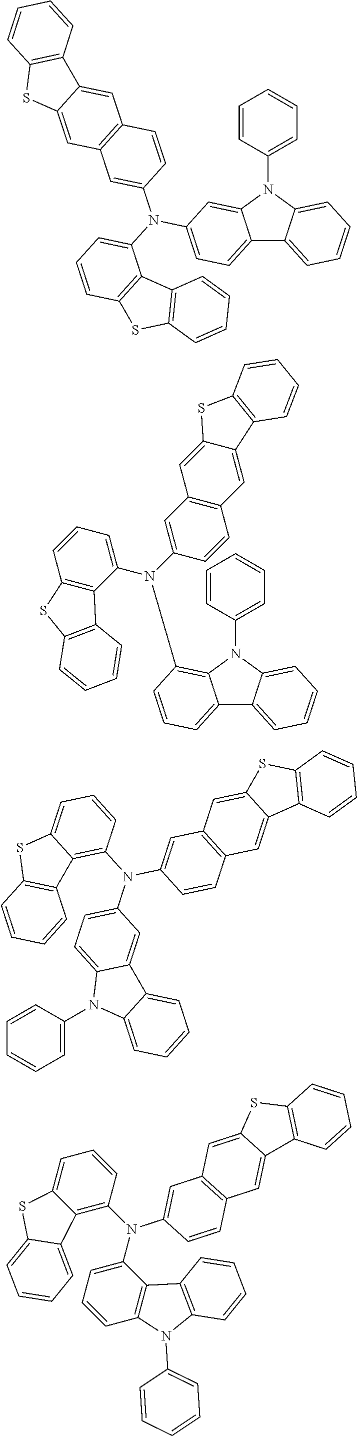 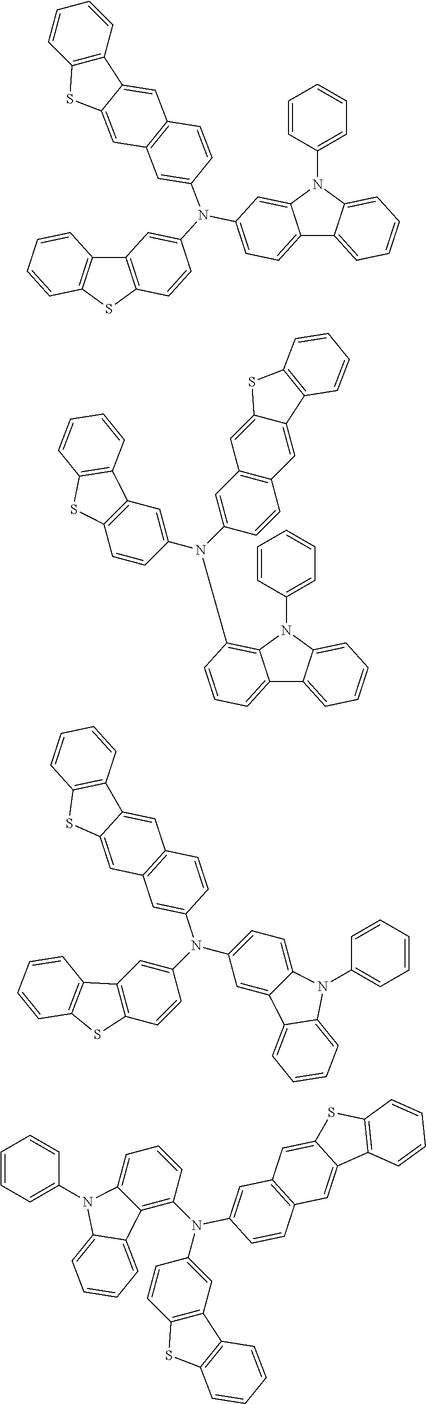 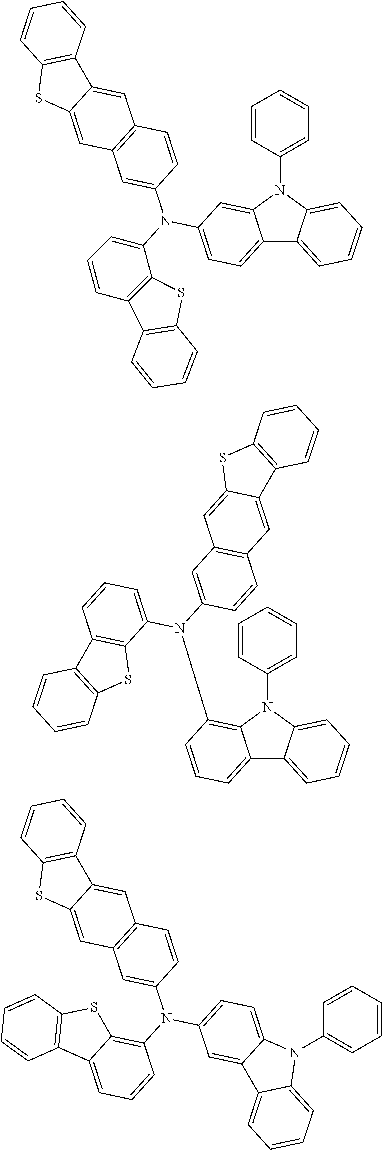 [SMILES: c1ccc(-n2c3ccccc3c3ccc(N(c4ccc5cc6c(cc5c4)sc4ccccc46)c4cccc5c4sc4ccccc45)cc32)cc1 ; c1ccc(-n2c3ccccc3c3cccc(N(c4ccc5cc6c(cc5c4)sc4ccccc46)c4cccc5c4sc4ccccc45)c32)cc1 ; c1ccc(-n2c3ccccc3c3cc(N(c4ccc5cc6c(cc5c4)sc4ccccc46)c4cccc5c4sc4ccccc45)ccc32)cc1] 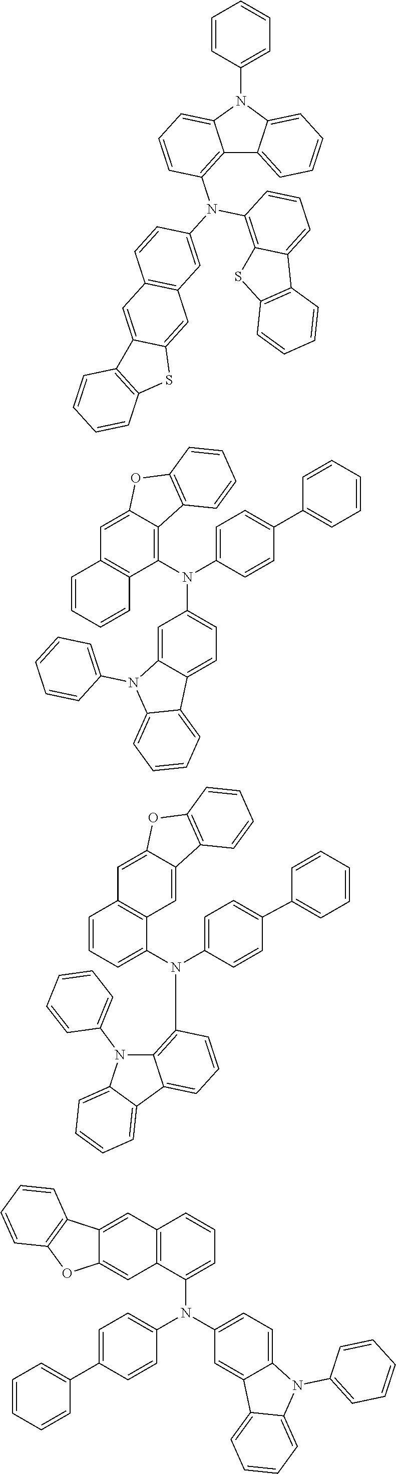 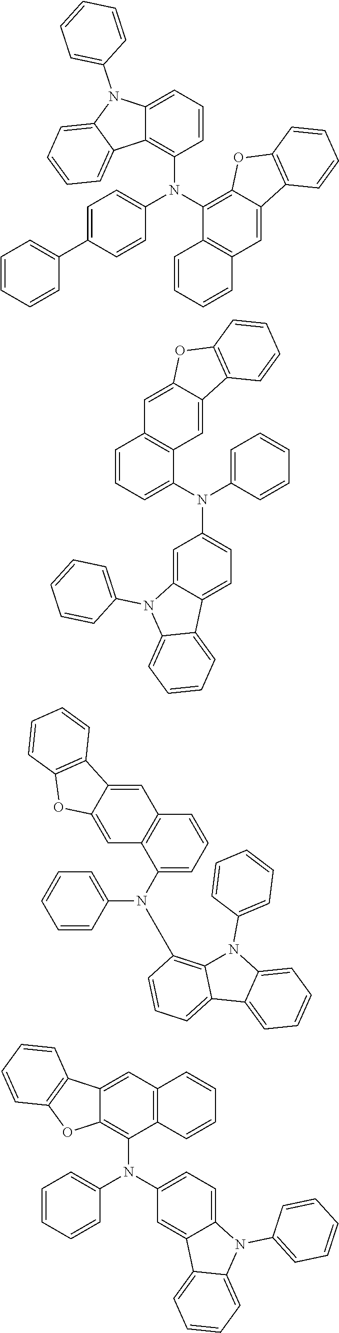 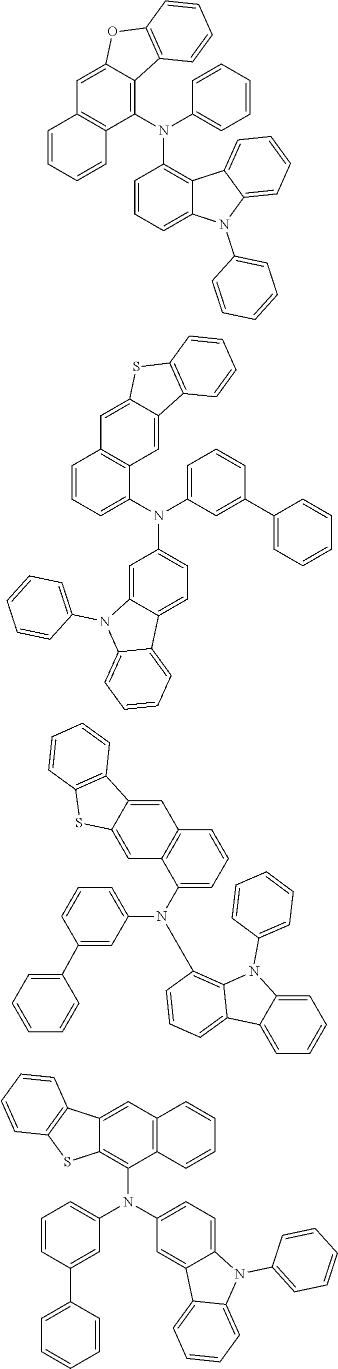 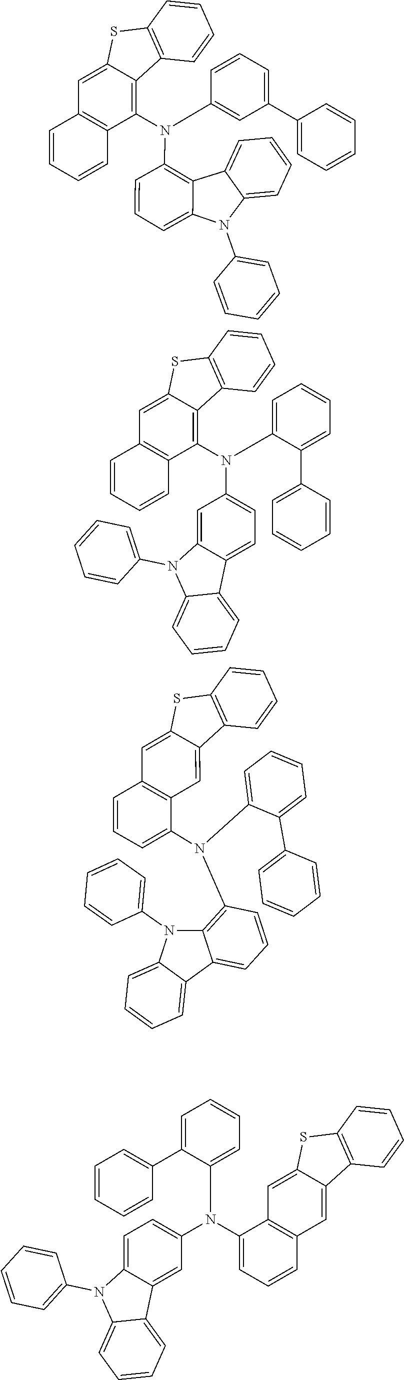 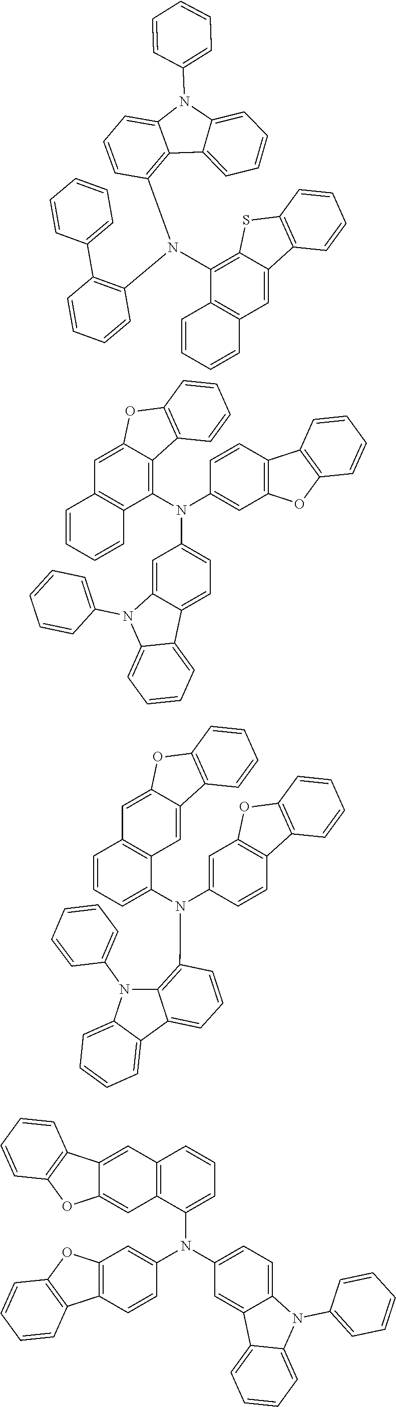 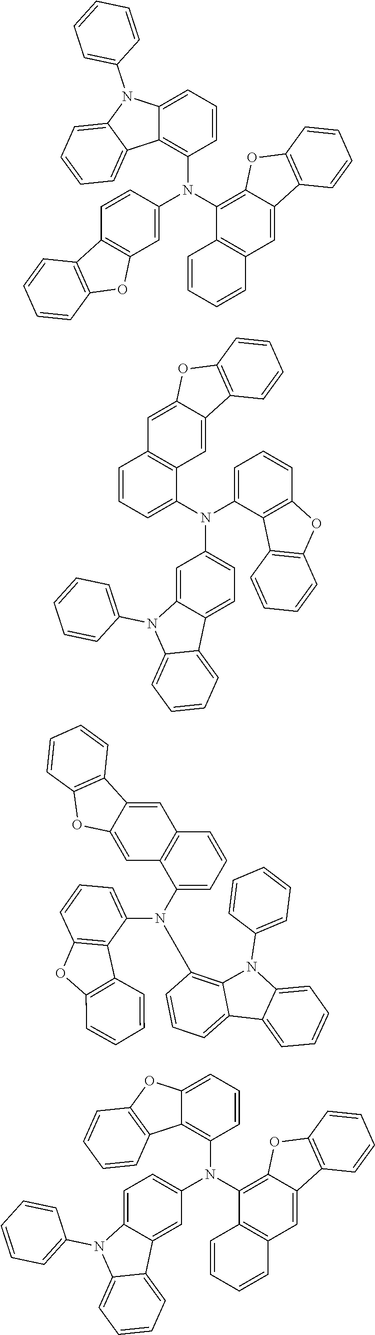 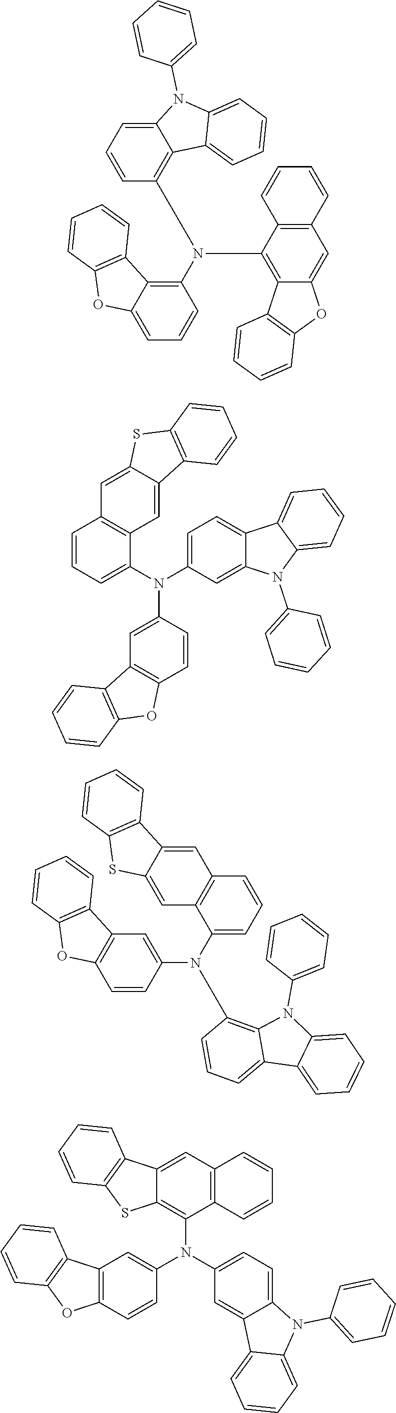 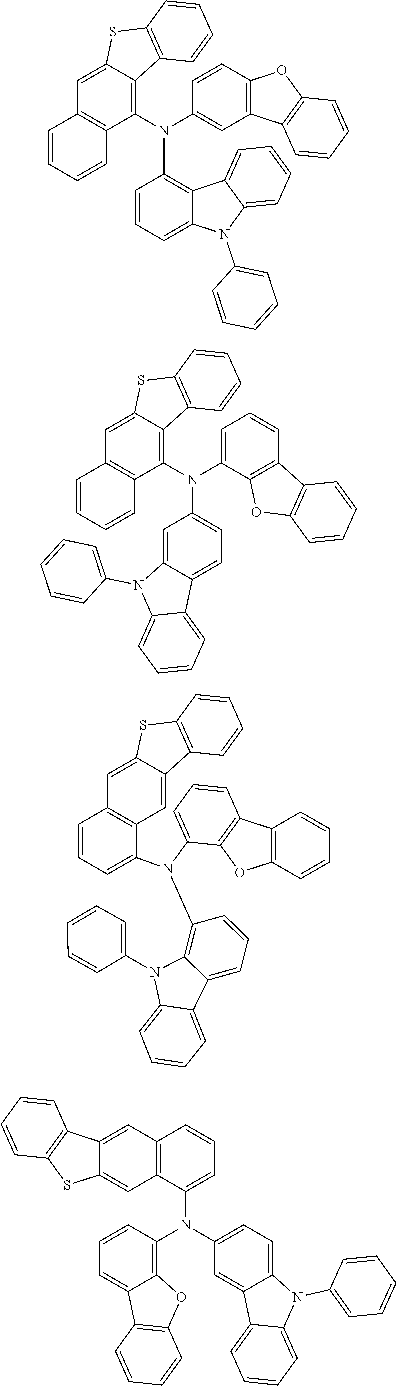 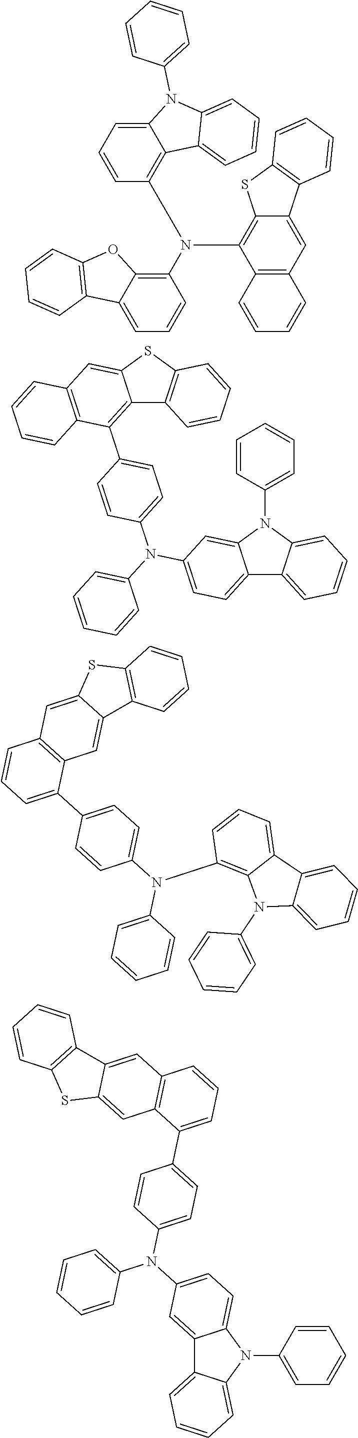 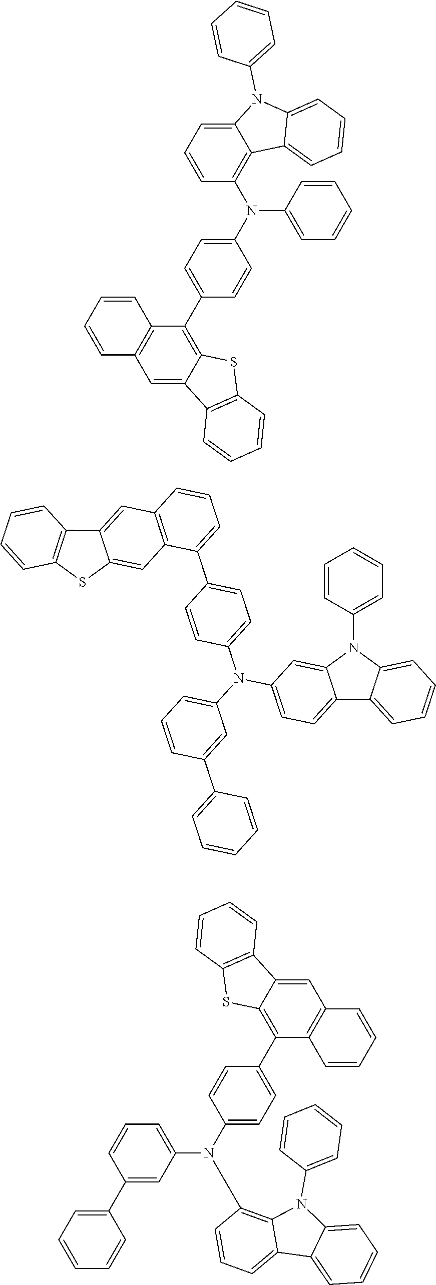 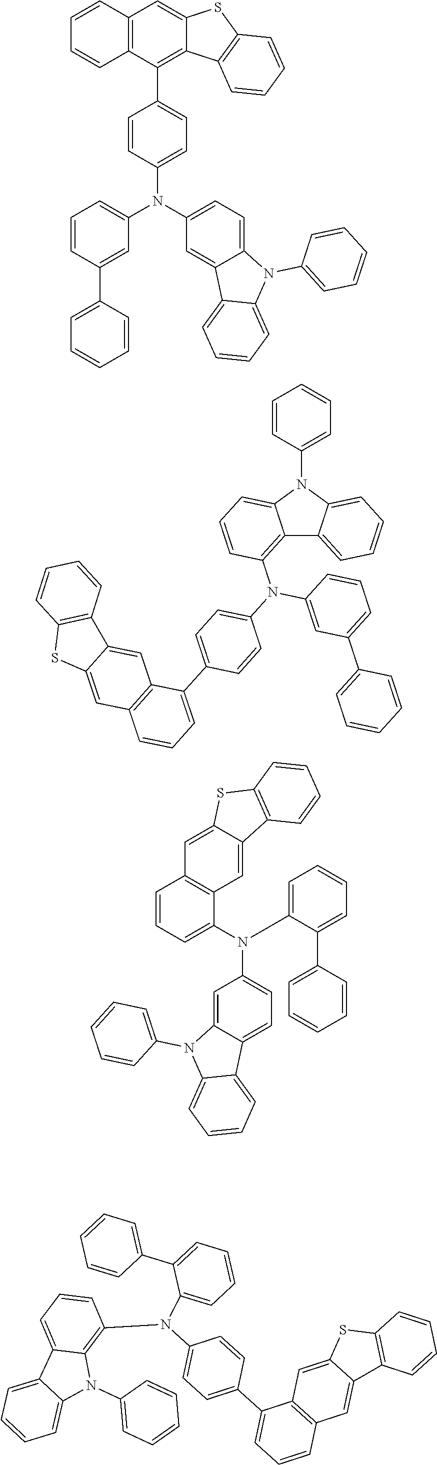 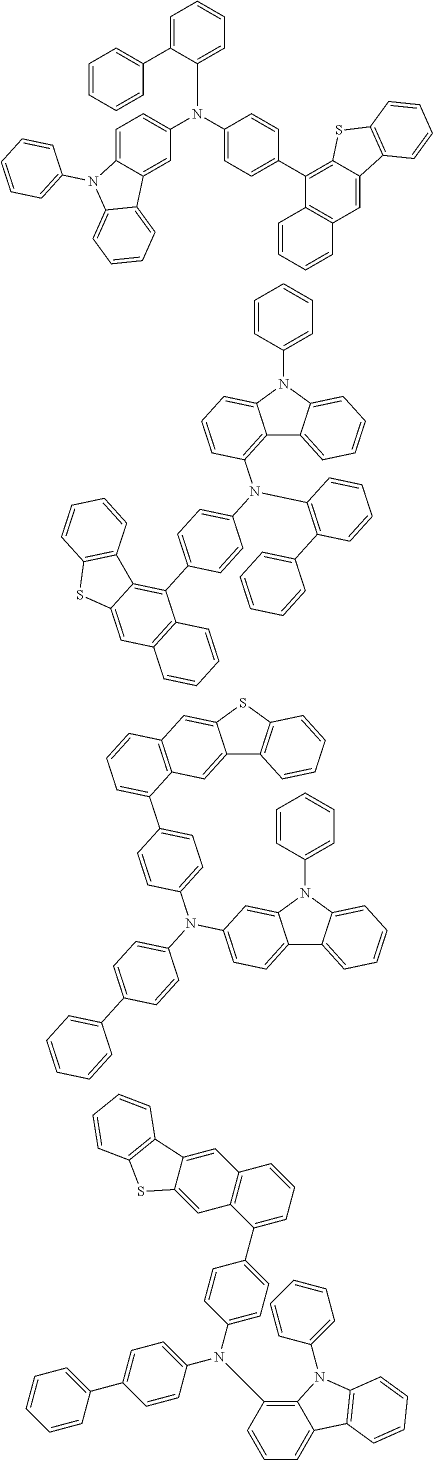 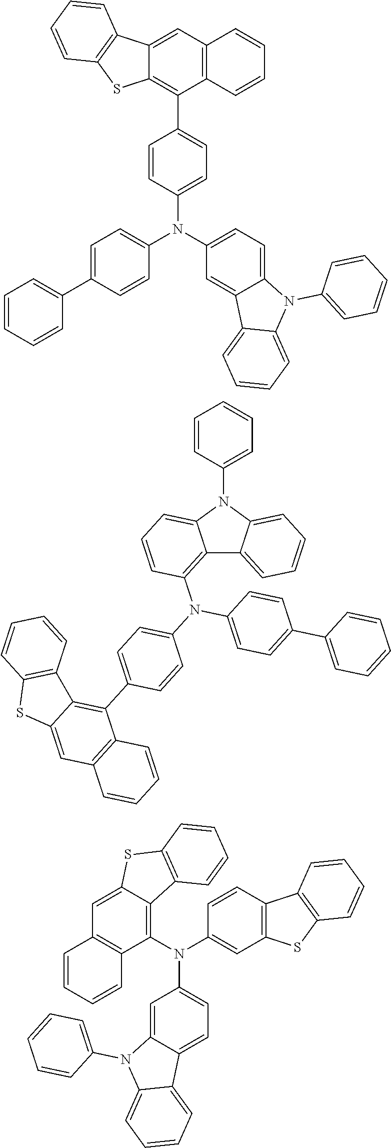 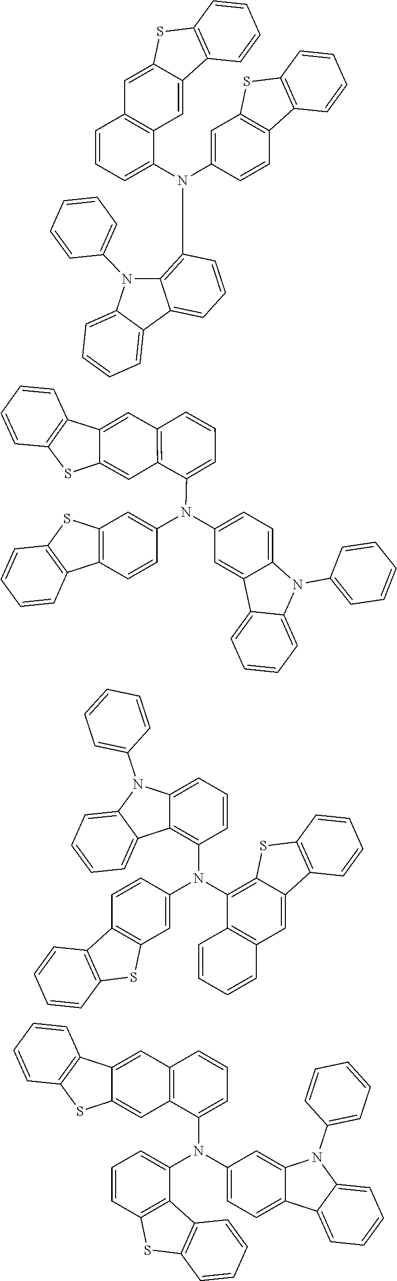 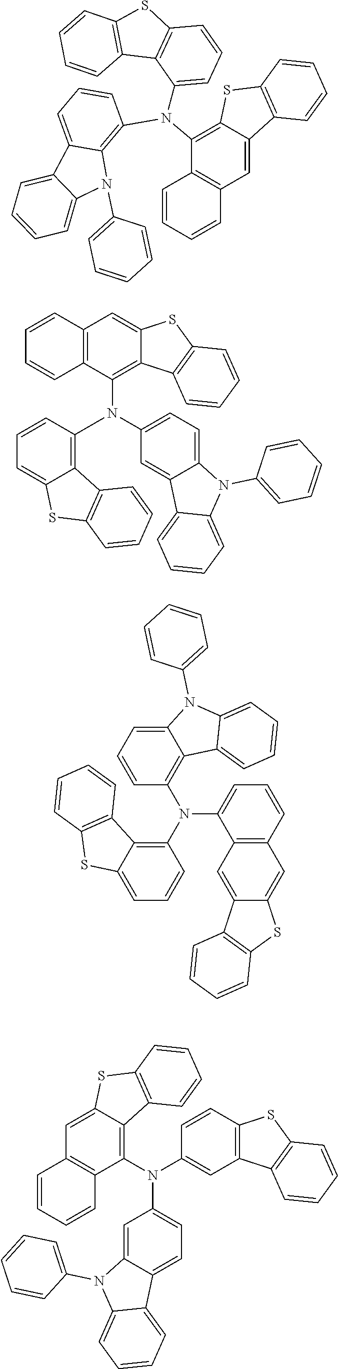 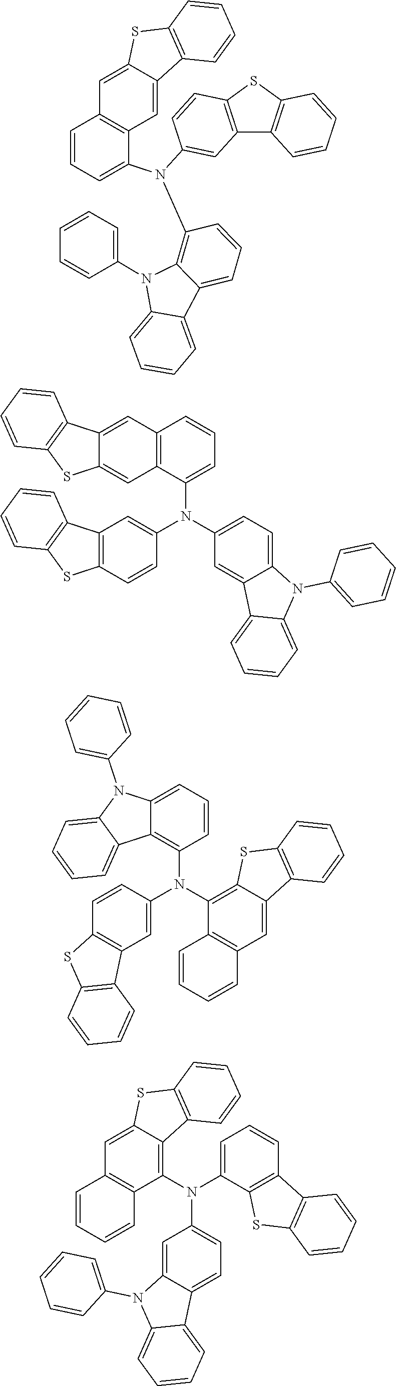 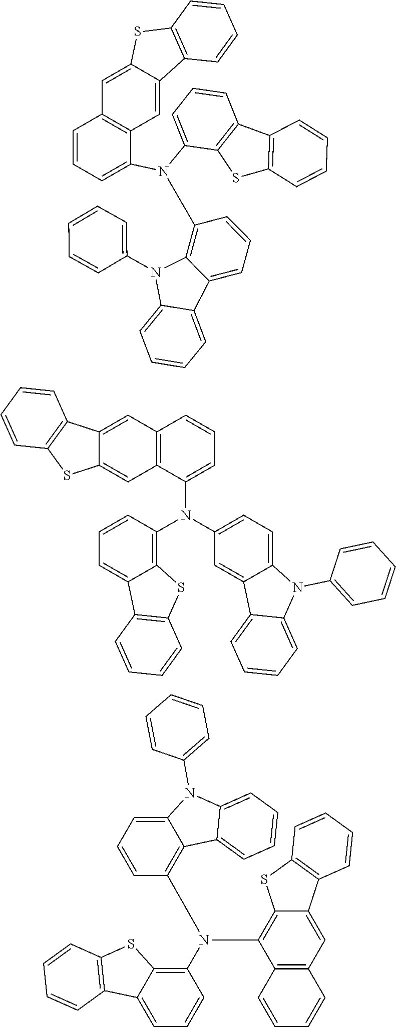 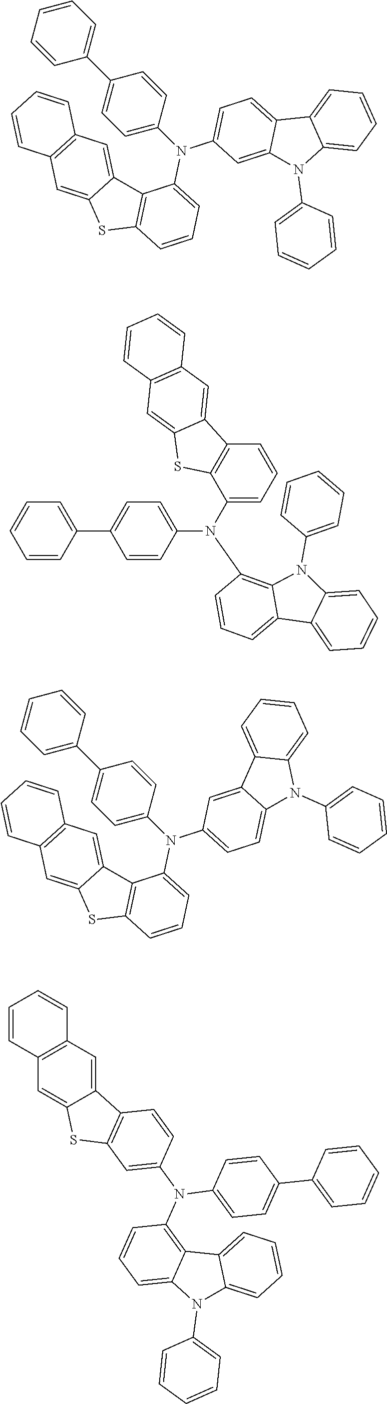 [SMILES: c1ccc(-c2ccc(N(c3ccc4c5ccccc5n(-c5ccccc5)c4c3)c3cccc4sc5cc6ccccc6cc5c34)cc2)cc1 ; c1ccc(-c2ccc(N(c3cccc4c3sc3cc5ccccc5cc34)c3cccc4c5ccccc5n(-c5ccccc5)c34)cc2)cc1 ; c1ccc(-c2ccc(N(c3ccc4c(c3)c3ccccc3n4-c3ccccc3)c3cccc4sc5cc6ccccc6cc5c34)cc2)cc1 ; c1ccc(-c2ccc(N(c3ccc4c(c3)sc3cc5ccccc5cc34)c3cccc4c3c3ccccc3n4-c3ccccc3)cc2)cc1] 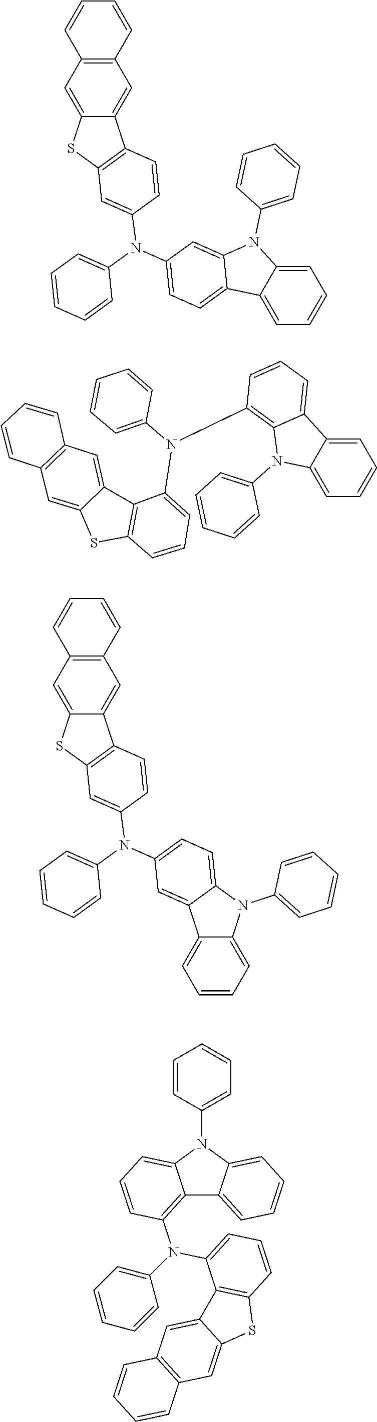 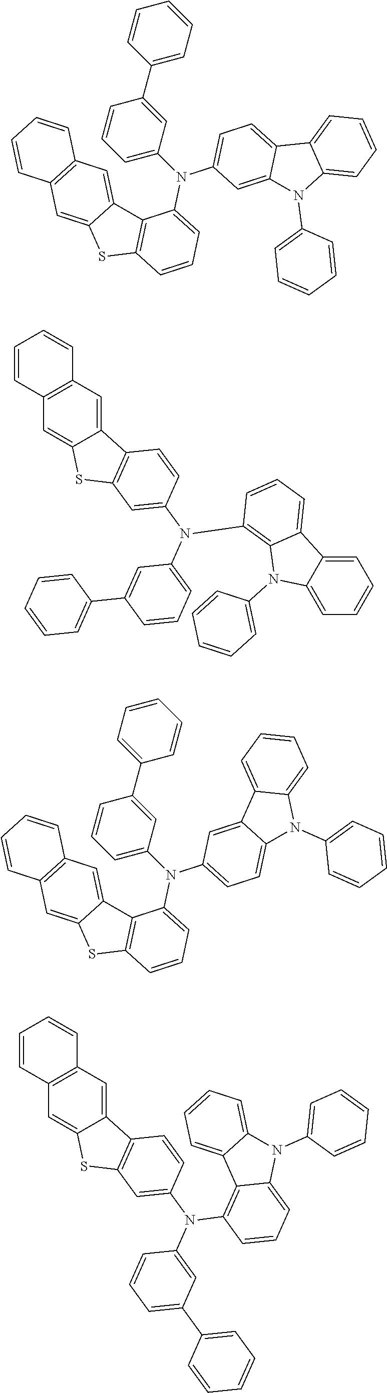 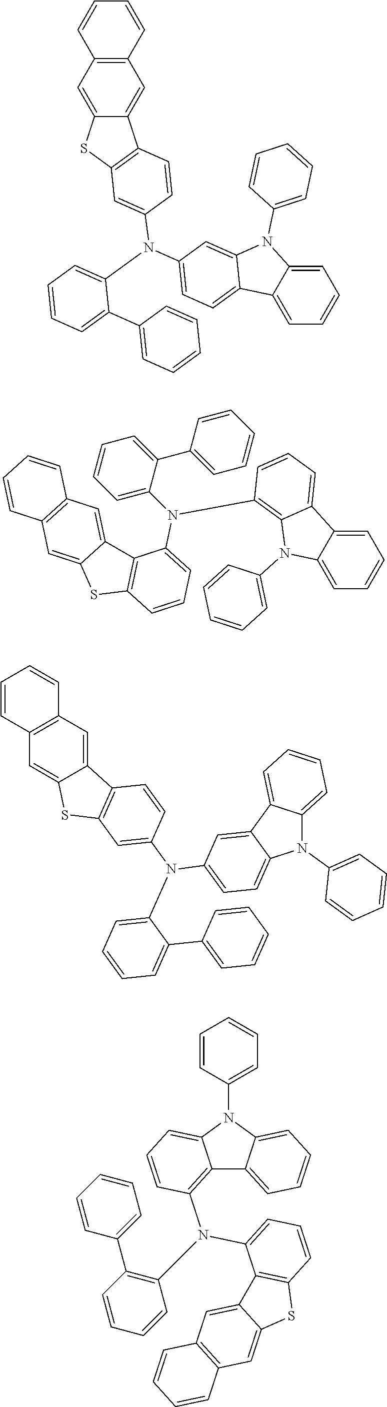 [SMILES: c1ccc(-c2ccccc2N(c2ccc3c(c2)sc2cc4ccccc4cc23)c2ccc3c4ccccc4n(-c4ccccc4)c3c2)cc1 ; c1ccc(-c2ccccc2N(c2cccc3sc4cc5ccccc5cc4c23)c2cccc3c4ccccc4n(-c4ccccc4)c23)cc1 ; c1ccc(-c2ccccc2N(c2ccc3c(c2)sc2cc4ccccc4cc23)c2ccc3c(c2)c2ccccc2n3-c2ccccc2)cc1 ; c1ccc(-c2ccccc2N(c2cccc3sc4cc5ccccc5cc4c23)c2cccc3c2c2ccccc2n3-c2ccccc2)cc1] 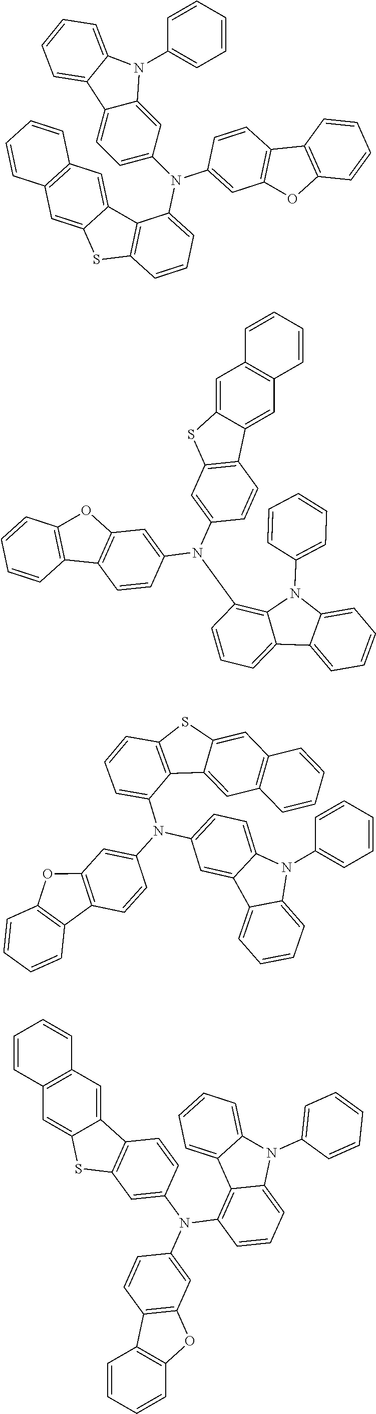 [SMILES: c1ccc(-n2c3ccccc3c3ccc(N(c4ccc5c(c4)oc4ccccc45)c4cccc5sc6cc7ccccc7cc6c45)cc32)cc1 ; c1ccc(-n2c3ccccc3c3cccc(N(c4ccc5c(c4)oc4ccccc45)c4ccc5c(c4)sc4cc6ccccc6cc45)c32)cc1 ; c1ccc(-n2c3ccccc3c3cc(N(c4ccc5c(c4)oc4ccccc45)c4cccc5sc6cc7ccccc7cc6c45)ccc32)cc1 ; c1ccc(-n2c3ccccc3c3c(N(c4ccc5c(c4)oc4ccccc45)c4ccc5c(c4)sc4cc6ccccc6cc45)cccc32)cc1] 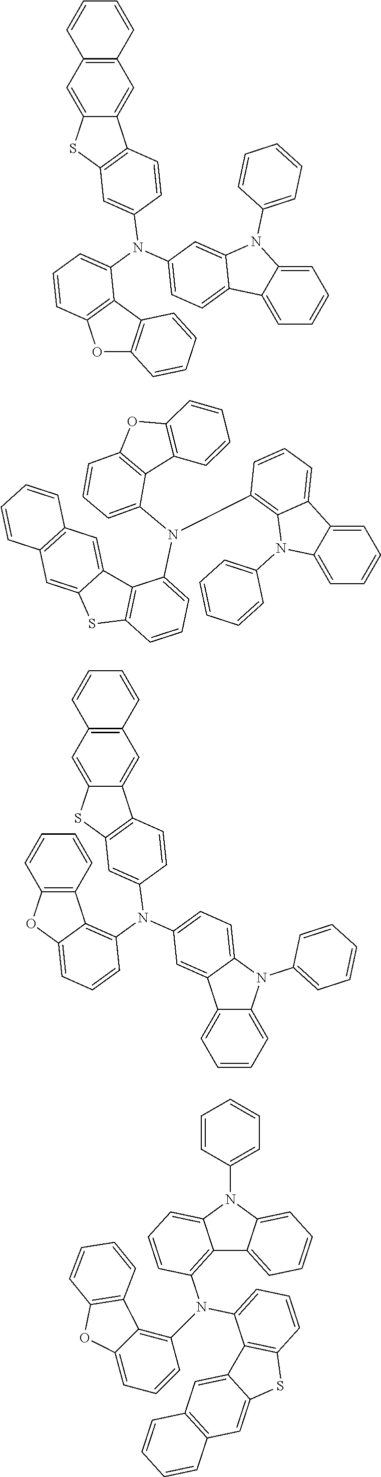 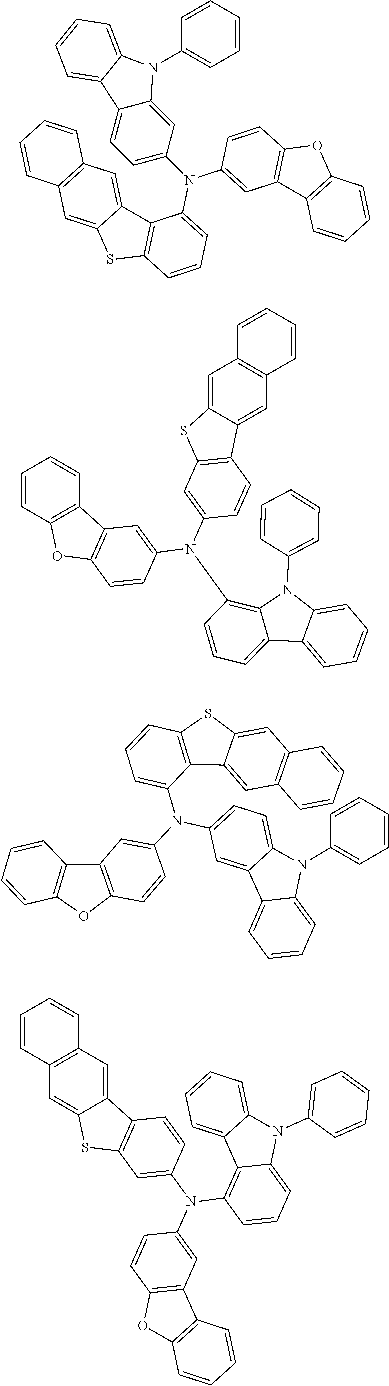 [SMILES: c1ccc(-n2c3ccccc3c3ccc(N(c4ccc5oc6ccccc6c5c4)c4cccc5sc6cc7ccccc7cc6c45)cc32)cc1 ; c1ccc(-n2c3ccccc3c3cccc(N(c4ccc5c(c4)sc4cc6ccccc6cc45)c4ccc5oc6ccccc6c5c4)c32)cc1 ; c1ccc(-n2c3ccccc3c3cc(N(c4ccc5oc6ccccc6c5c4)c4cccc5sc6cc7ccccc7cc6c45)ccc32)cc1 ; c1ccc(-n2c3ccccc3c3c(N(c4ccc5c(c4)sc4cc6ccccc6cc45)c4ccc5oc6ccccc6c5c4)cccc32)cc1] 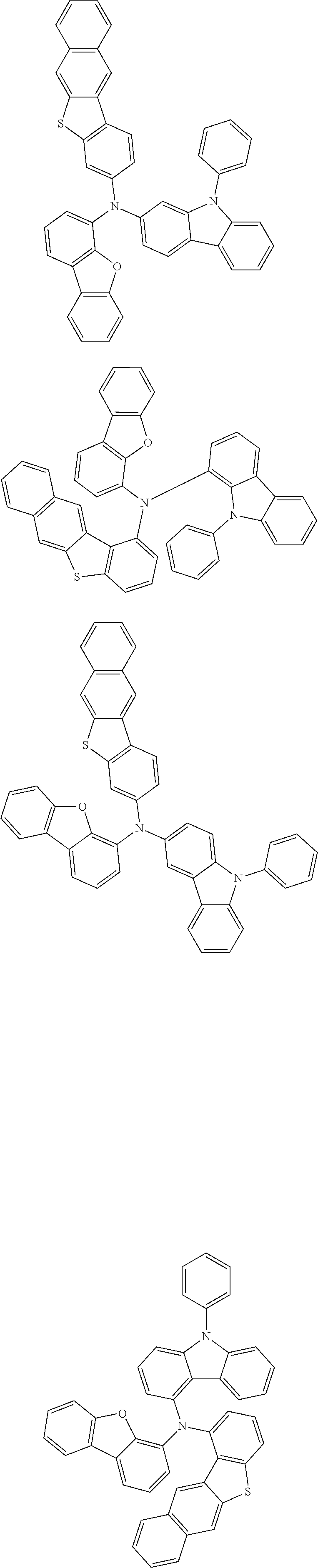 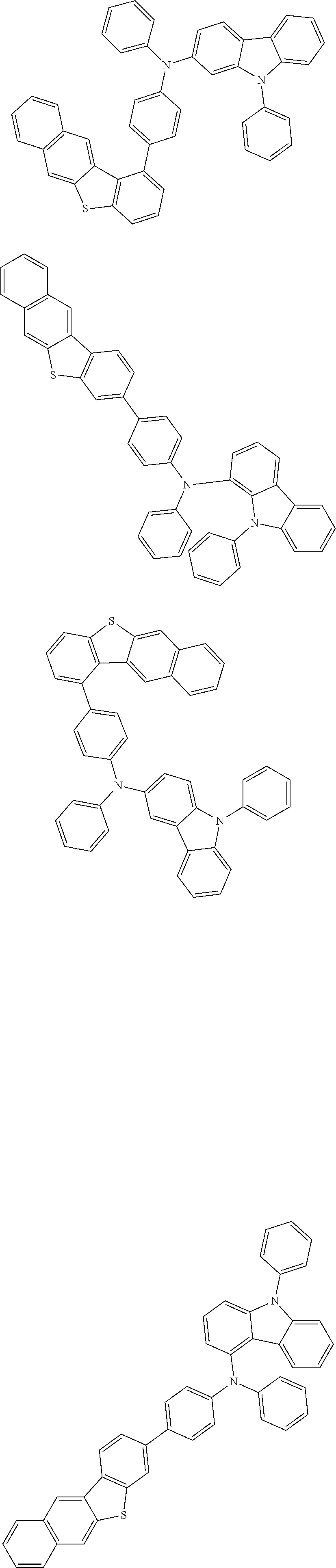 [SMILES: c1ccc(N(c2ccc(-c3cccc4sc5cc6ccccc6cc5c34)cc2)c2ccc3c4ccccc4n(-c4ccccc4)c3c2)cc1 ; c1ccc(N(c2ccc(-c3ccc4c(c3)sc3cc5ccccc5cc34)cc2)c2cccc3c4ccccc4n(-c4ccccc4)c23)cc1 ; c1ccc(N(c2ccc(-c3cccc4sc5cc6ccccc6cc5c34)cc2)c2ccc3c(c2)c2ccccc2n3-c2ccccc2)cc1 ; c1ccc(N(c2ccc(-c3ccc4c(c3)sc3cc5ccccc5cc34)cc2)c2cccc3c2c2ccccc2n3-c2ccccc2)cc1] 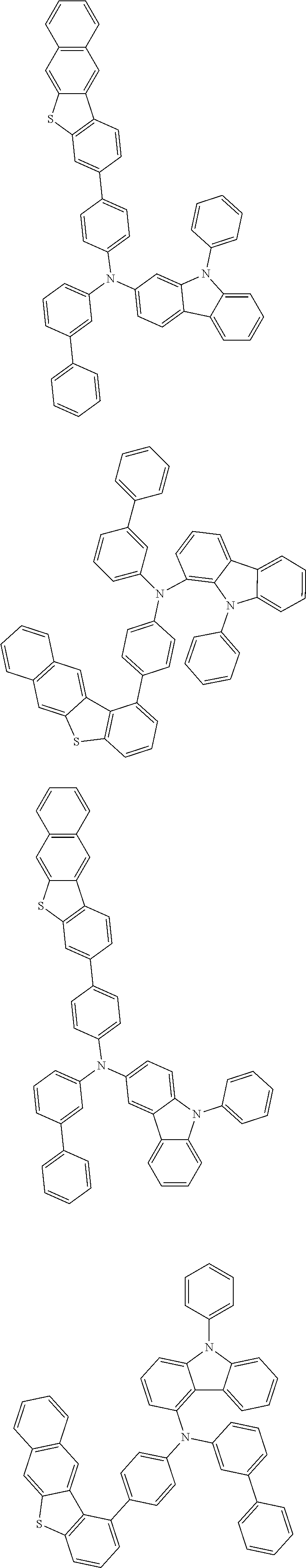 [SMILES: c1ccc(-c2cccc(N(c3ccc(-c4ccc5c(c4)sc4cc6ccccc6cc45)cc3)c3ccc4c5ccccc5n(-c5ccccc5)c4c3)c2)cc1 ; c1ccc(-c2cccc(N(c3ccc(-c4cccc5sc6cc7ccccc7cc6c45)cc3)c3cccc4c5ccccc5n(-c5ccccc5)c34)c2)cc1 ; c1ccc(-c2cccc(N(c3ccc(-c4ccc5c(c4)sc4cc6ccccc6cc45)cc3)c3ccc4c(c3)c3ccccc3n4-c3ccccc3)c2)cc1 ; c1ccc(-c2cccc(N(c3ccc(-c4cccc5sc6cc7ccccc7cc6c45)cc3)c3cccc4c3c3ccccc3n4-c3ccccc3)c2)cc1] 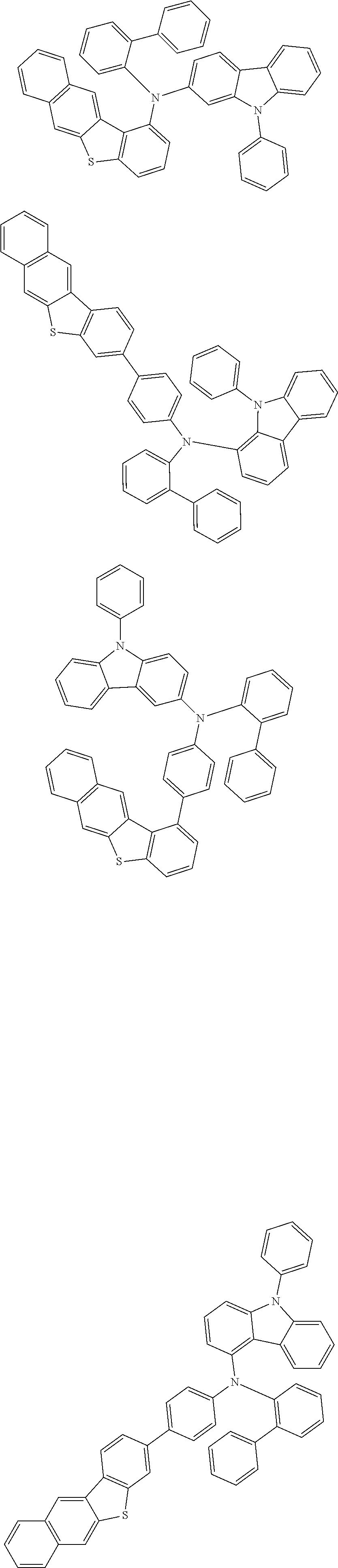 [SMILES: c1ccc(-c2ccccc2N(c2ccc3c4ccccc4n(-c4ccccc4)c3c2)c2cccc3sc4cc5ccccc5cc4c23)cc1 ; c1ccc(-c2ccccc2N(c2ccc(-c3ccc4c(c3)sc3cc5ccccc5cc34)cc2)c2cccc3c4ccccc4n(-c4ccccc4)c23)cc1 ; c1ccc(-c2ccccc2N(c2ccc(-c3cccc4sc5cc6ccccc6cc5c34)cc2)c2ccc3c(c2)c2ccccc2n3-c2ccccc2)cc1 ; c1ccc(-c2ccccc2N(c2ccc(-c3ccc4c(c3)sc3cc5ccccc5cc34)cc2)c2cccc3c2c2ccccc2n3-c2ccccc2)cc1] 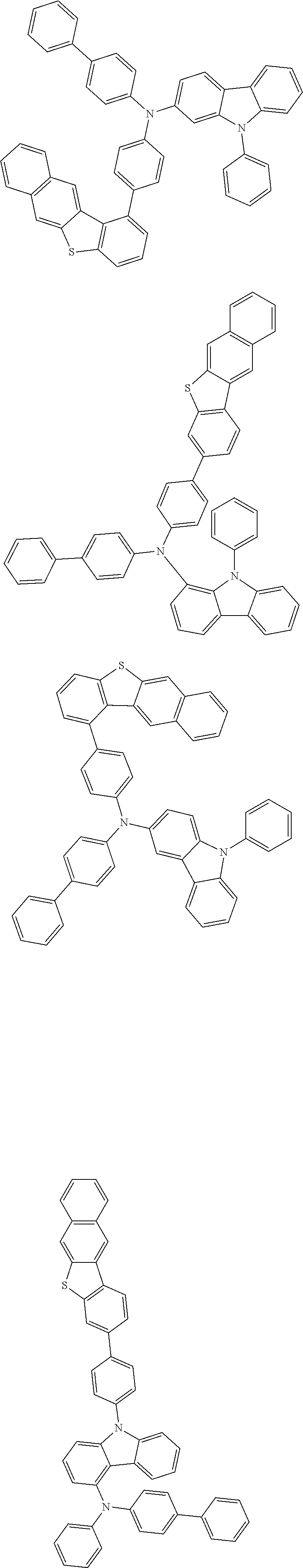 [SMILES: c1ccc(-c2ccc(N(c3ccc(-c4cccc5sc6cc7ccccc7cc6c45)cc3)c3ccc4c5ccccc5n(-c5ccccc5)c4c3)cc2)cc1 ; c1ccc(-c2ccc(N(c3ccc(-c4ccc5c(c4)sc4cc6ccccc6cc45)cc3)c3cccc4c5ccccc5n(-c5ccccc5)c34)cc2)cc1 ; c1ccc(-c2ccc(N(c3ccc(-c4cccc5sc6cc7ccccc7cc6c45)cc3)c3ccc4c(c3)c3ccccc3n4-c3ccccc3)cc2)cc1 ; c1ccc(-c2ccc(N(c3ccccc3)c3cccc4c3c3ccccc3n4-c3ccc(-c4ccc5c(c4)sc4cc6ccccc6cc45)cc3)cc2)cc1] 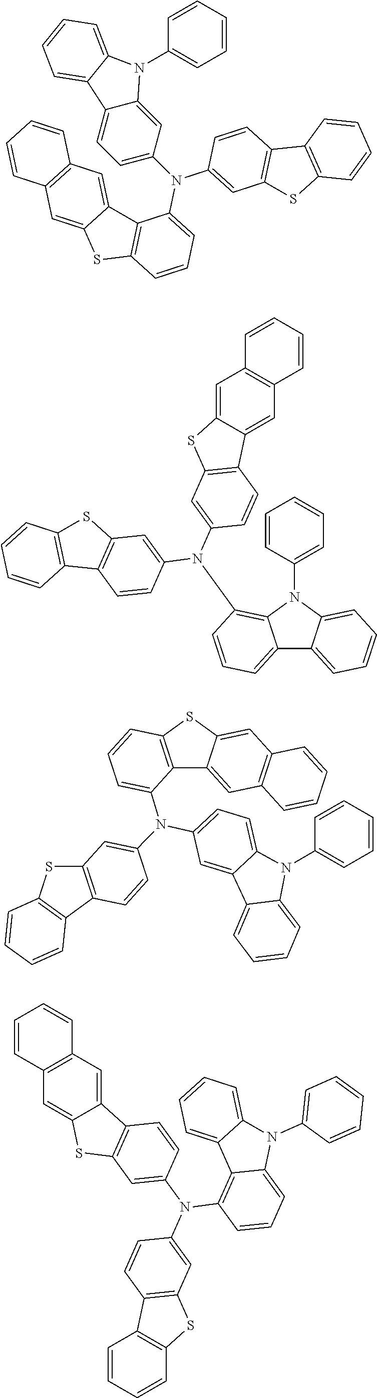 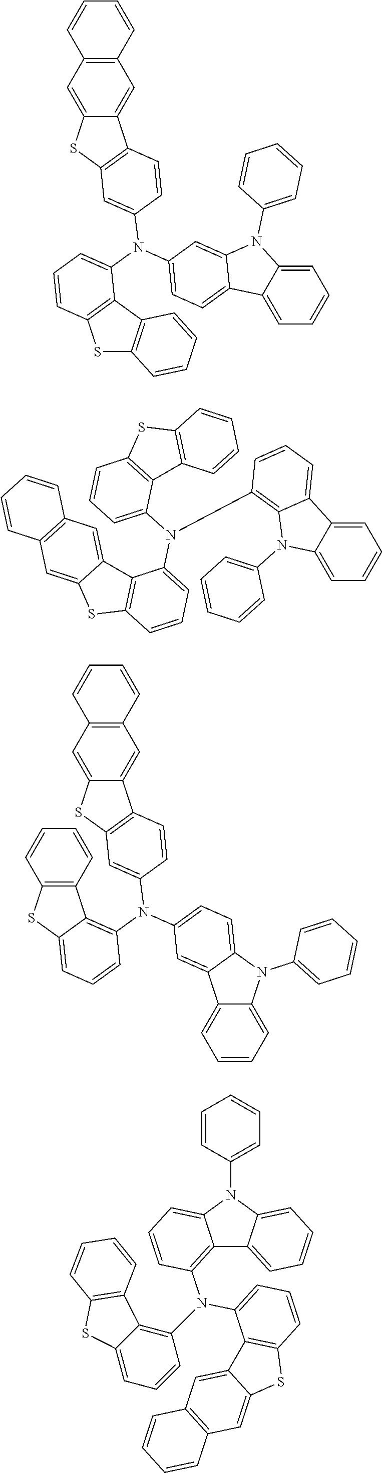 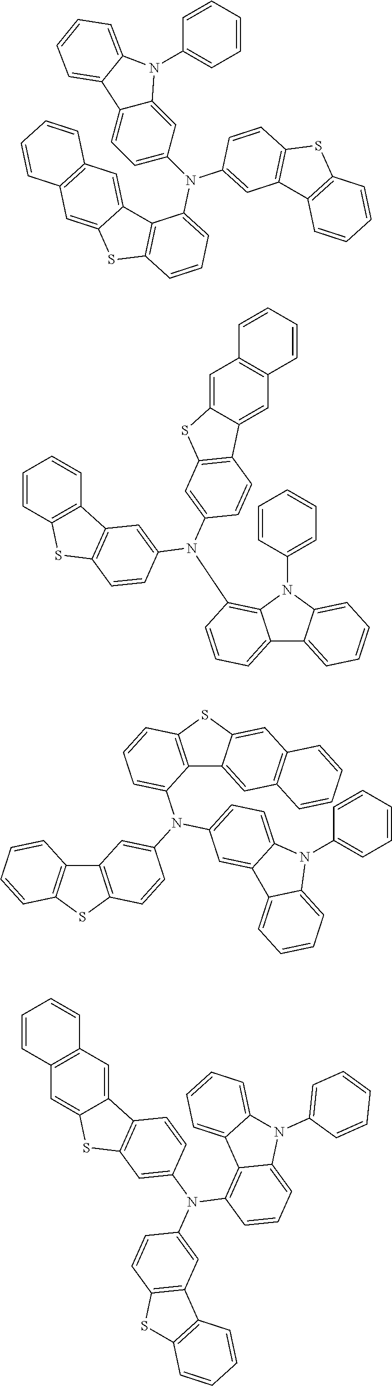 [SMILES: c1ccc(-n2c3ccccc3c3ccc(N(c4ccc5sc6ccccc6c5c4)c4cccc5sc6cc7ccccc7cc6c45)cc32)cc1 ; c1ccc(-n2c3ccccc3c3cccc(N(c4ccc5c(c4)sc4cc6ccccc6cc45)c4ccc5sc6ccccc6c5c4)c32)cc1 ; c1ccc(-n2c3ccccc3c3cc(N(c4ccc5sc6ccccc6c5c4)c4cccc5sc6cc7ccccc7cc6c45)ccc32)cc1 ; c1ccc(-n2c3ccccc3c3c(N(c4ccc5c(c4)sc4cc6ccccc6cc45)c4ccc5sc6ccccc6c5c4)cccc32)cc1] 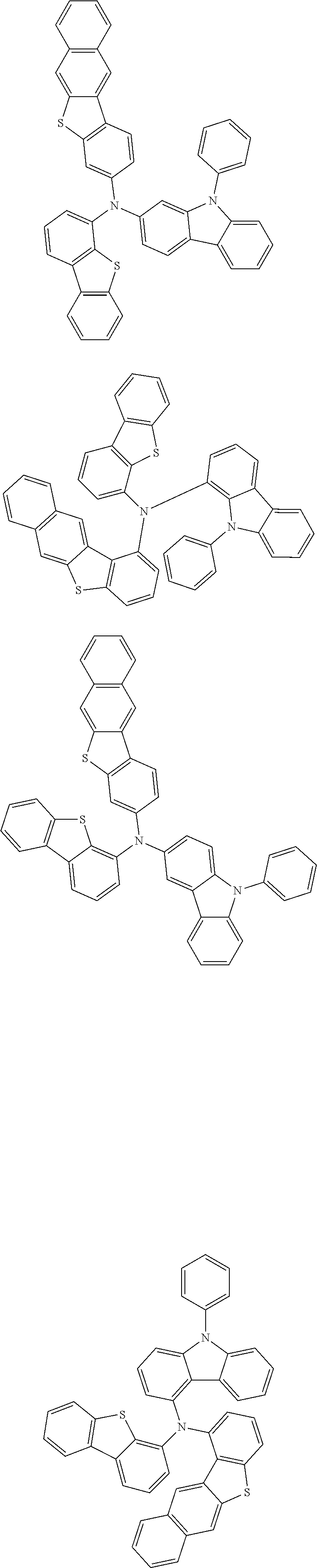 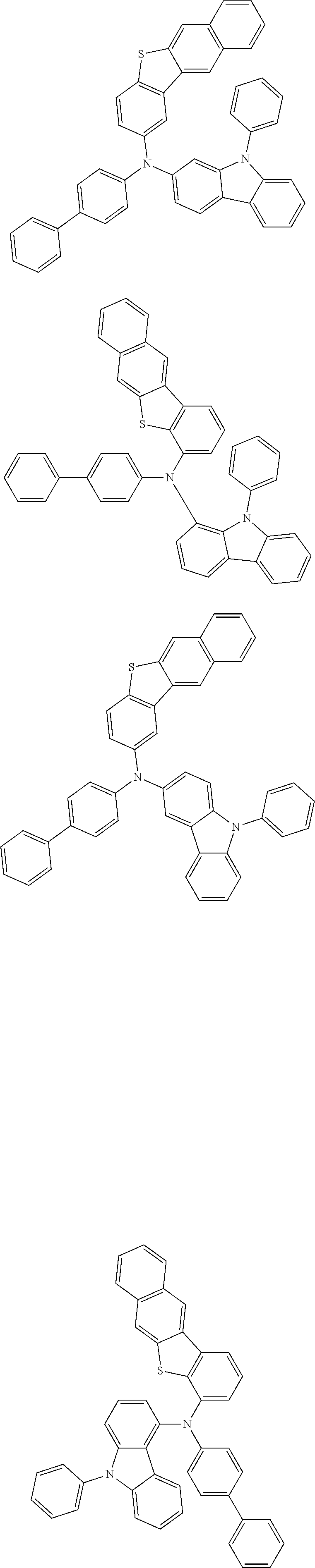 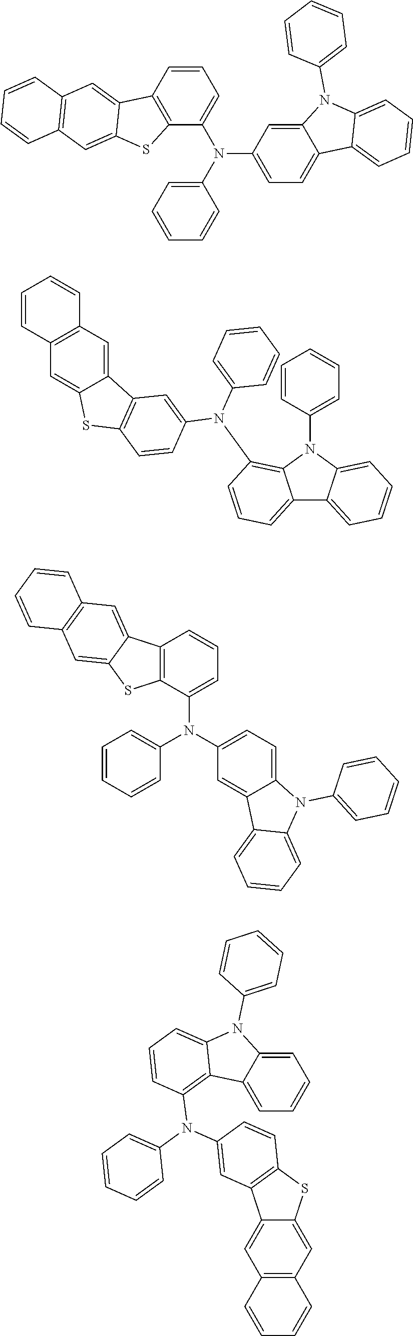 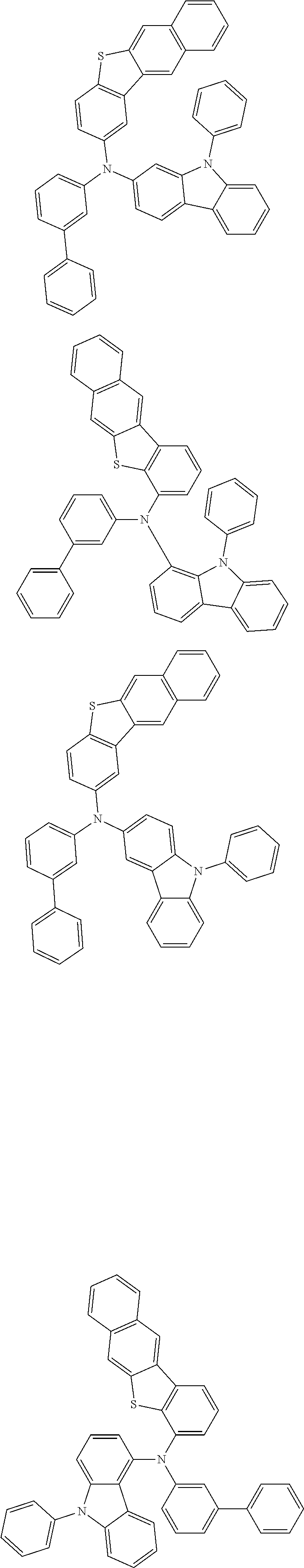 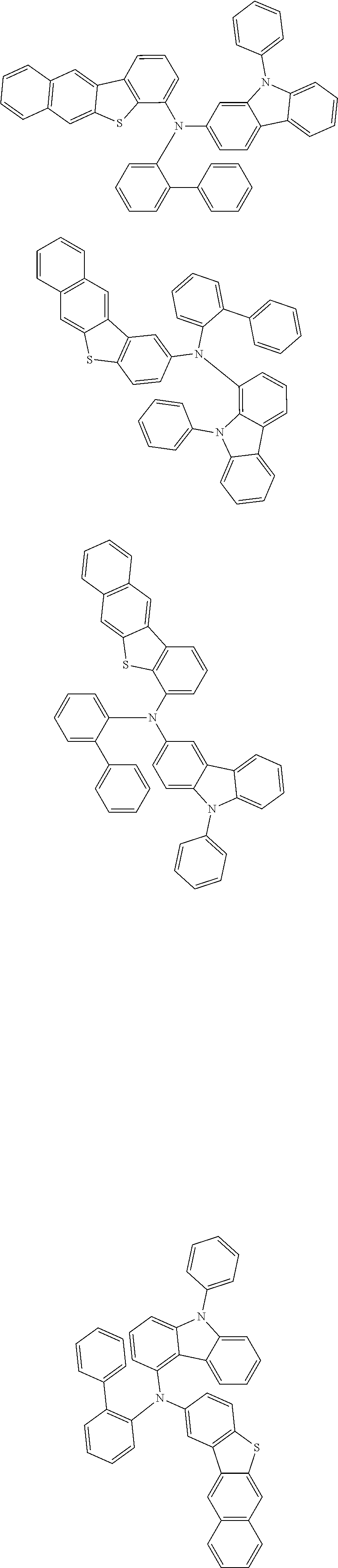 [SMILES: c1ccc(-c2ccccc2N(c2ccc3c4ccccc4n(-c4ccccc4)c3c2)c2cccc3c2sc2cc4ccccc4cc23)cc1 ; c1ccc(-c2ccccc2N(c2ccc3sc4cc5ccccc5cc4c3c2)c2cccc3c4ccccc4n(-c4ccccc4)c23)cc1 ; c1ccc(-c2ccccc2N(c2ccc3c(c2)c2ccccc2n3-c2ccccc2)c2cccc3c2sc2cc4ccccc4cc23)cc1 ; c1ccc(-c2ccccc2N(c2ccc3sc4cc5ccccc5cc4c3c2)c2cccc3c2c2ccccc2n3-c2ccccc2)cc1] 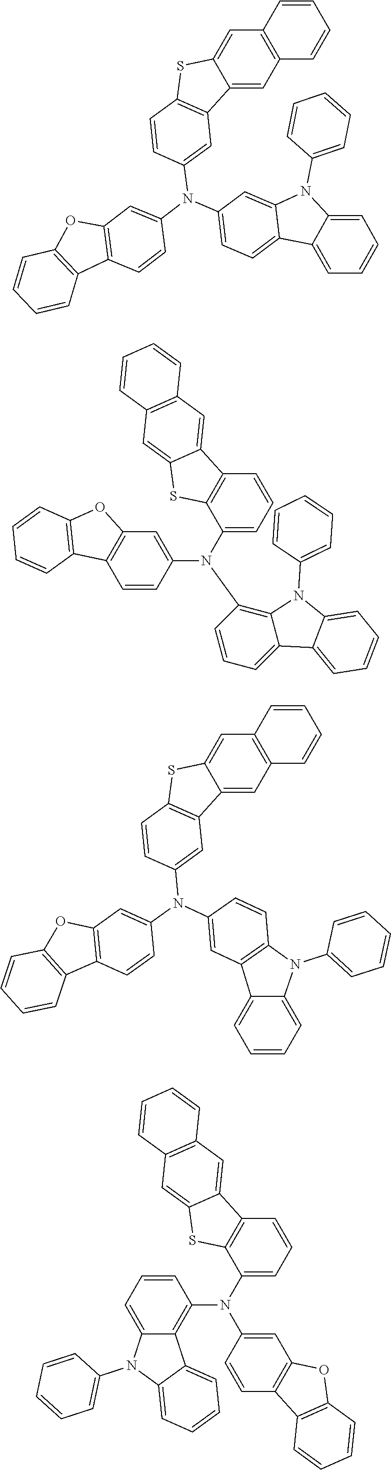 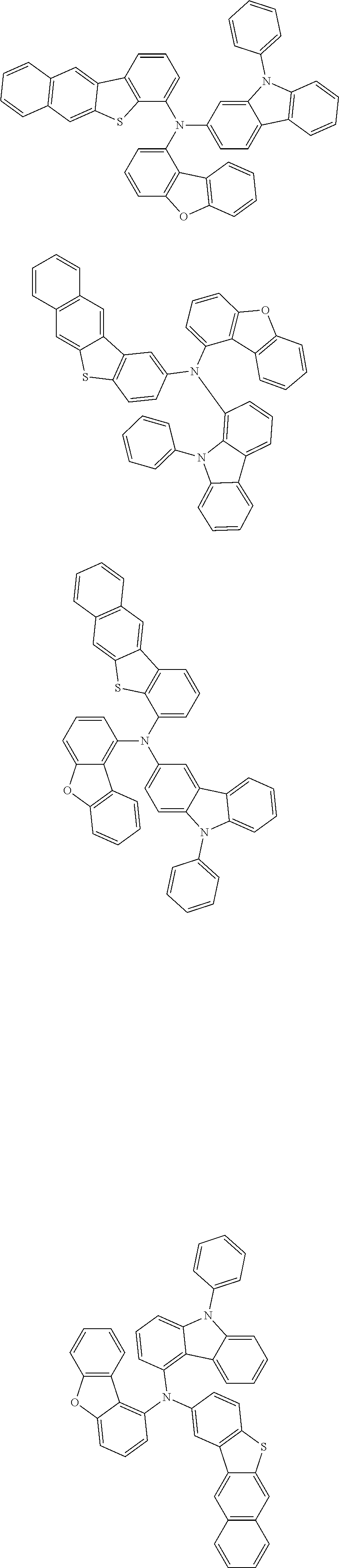 [SMILES: c1ccc(-n2c3ccccc3c3ccc(N(c4cccc5c4sc4cc6ccccc6cc45)c4cccc5oc6ccccc6c45)cc32)cc1 ; c1ccc(-n2c3ccccc3c3cccc(N(c4ccc5sc6cc7ccccc7cc6c5c4)c4cccc5oc6ccccc6c45)c32)cc1 ; c1ccc(-n2c3ccccc3c3cc(N(c4cccc5c4sc4cc6ccccc6cc45)c4cccc5oc6ccccc6c45)ccc32)cc1 ; c1ccc(-n2c3ccccc3c3c(N(c4ccc5sc6cc7ccccc7cc6c5c4)c4cccc5oc6ccccc6c45)cccc32)cc1] 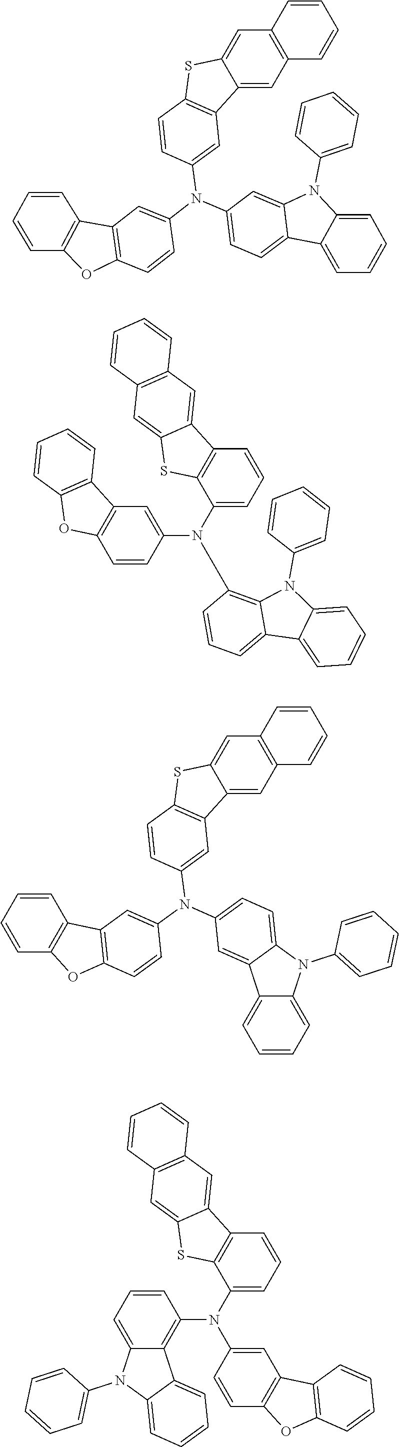 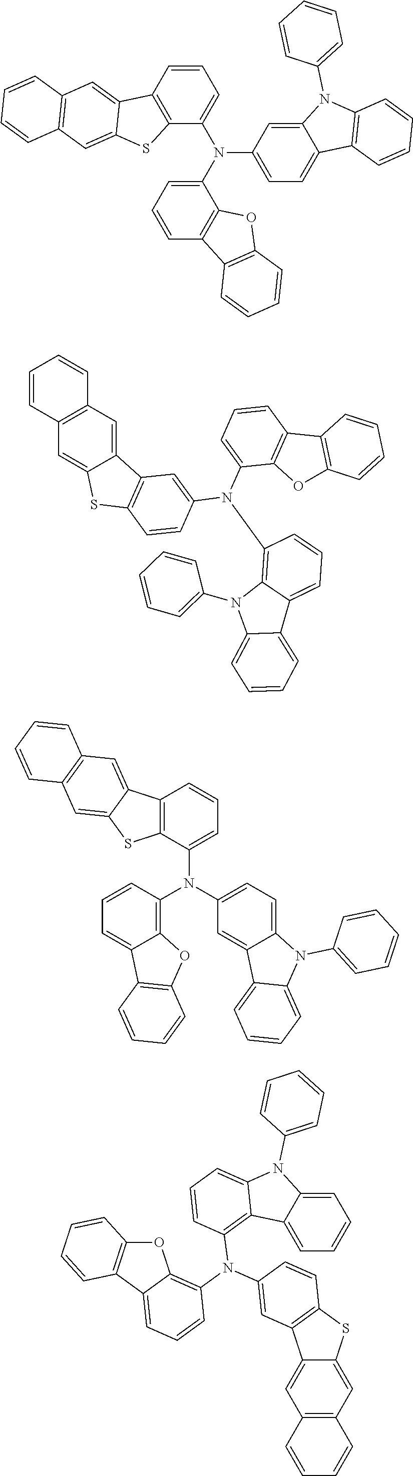 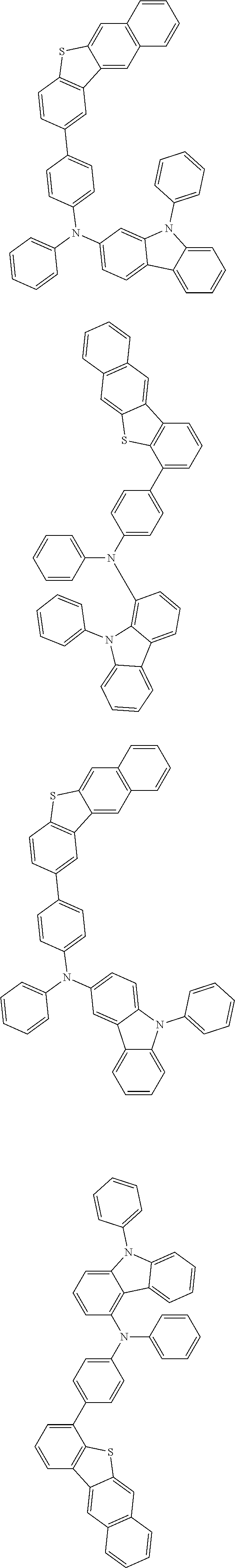 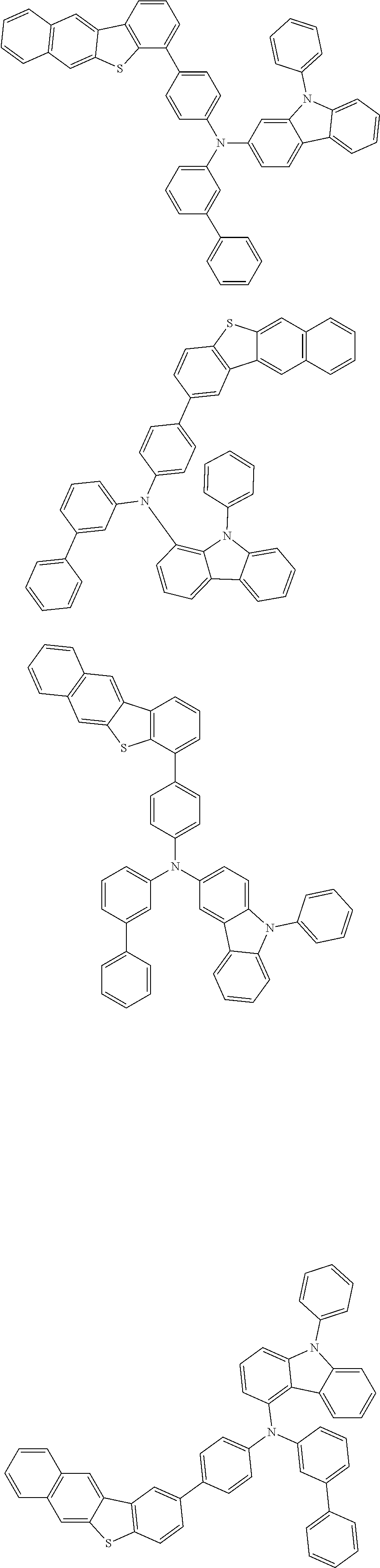 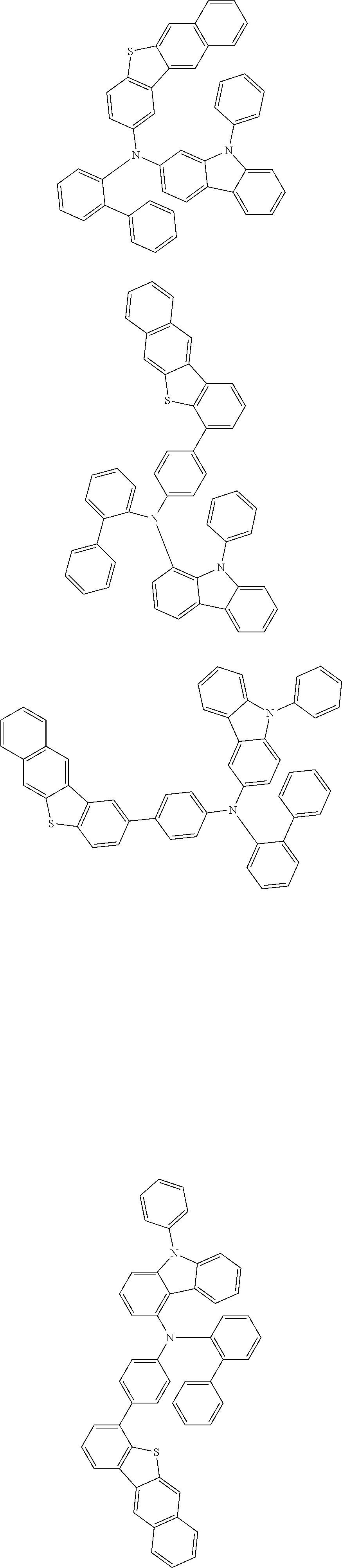 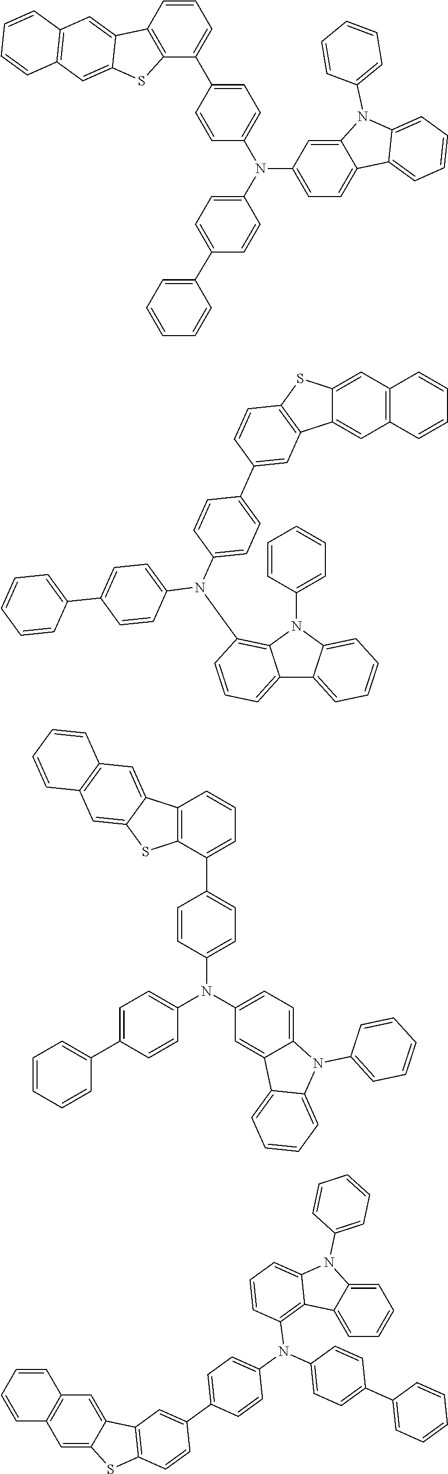 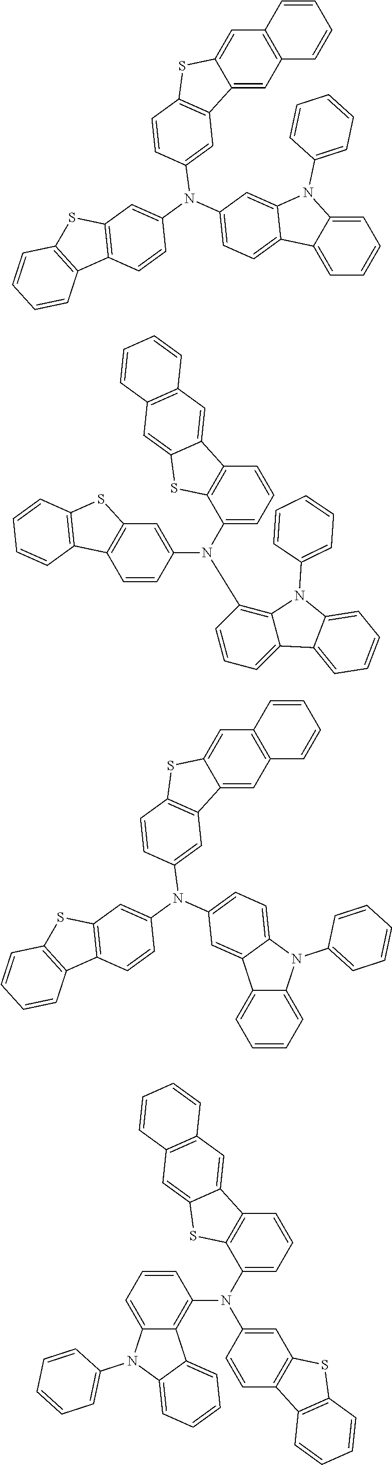 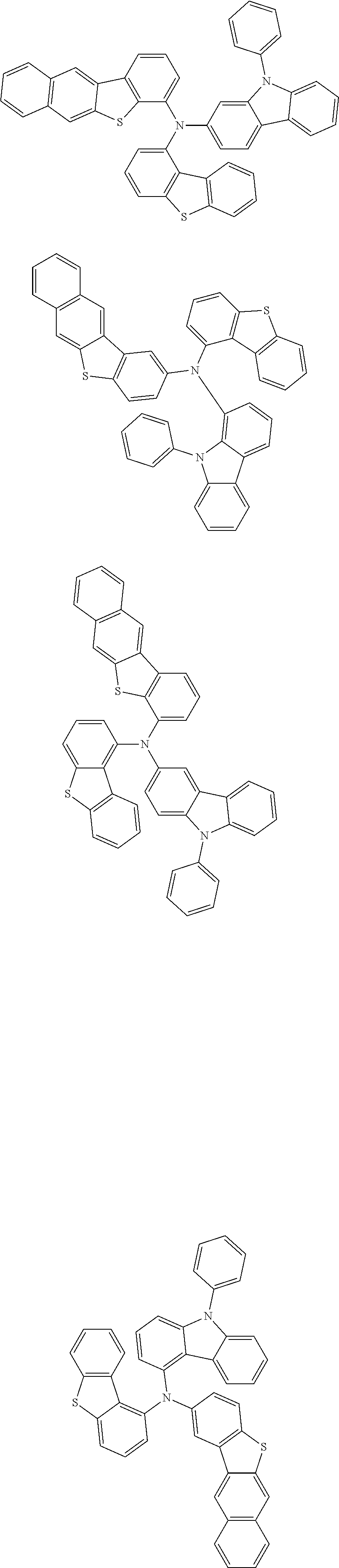 [SMILES: c1ccc(-n2c3ccccc3c3ccc(N(c4cccc5c4sc4cc6ccccc6cc45)c4cccc5sc6ccccc6c45)cc32)cc1 ; c1ccc(-n2c3ccccc3c3cccc(N(c4ccc5sc6cc7ccccc7cc6c5c4)c4cccc5sc6ccccc6c45)c32)cc1 ; c1ccc(-n2c3ccccc3c3cc(N(c4cccc5c4sc4cc6ccccc6cc45)c4cccc5sc6ccccc6c45)ccc32)cc1 ; c1ccc(-n2c3ccccc3c3c(N(c4ccc5sc6cc7ccccc7cc6c5c4)c4cccc5sc6ccccc6c45)cccc32)cc1] 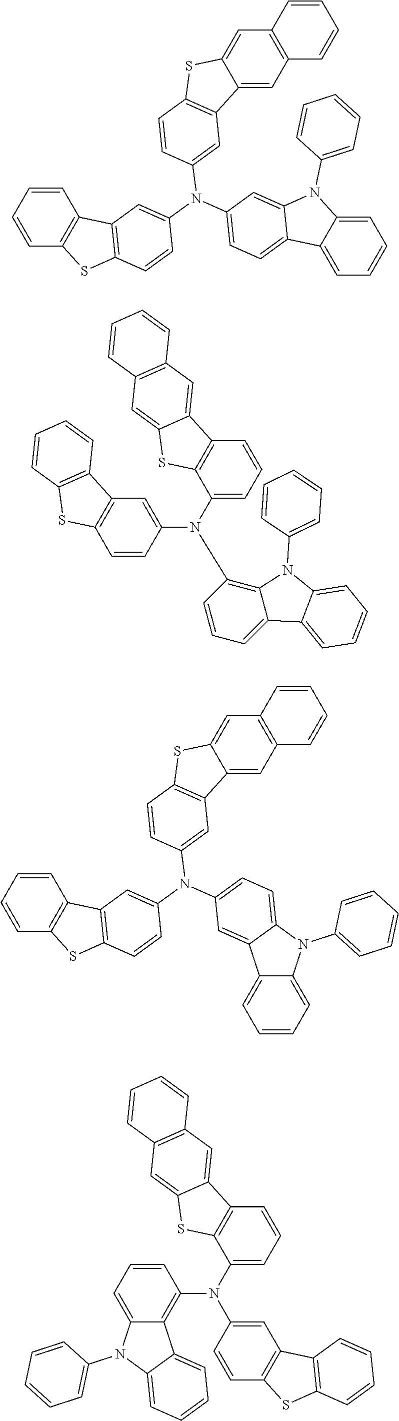 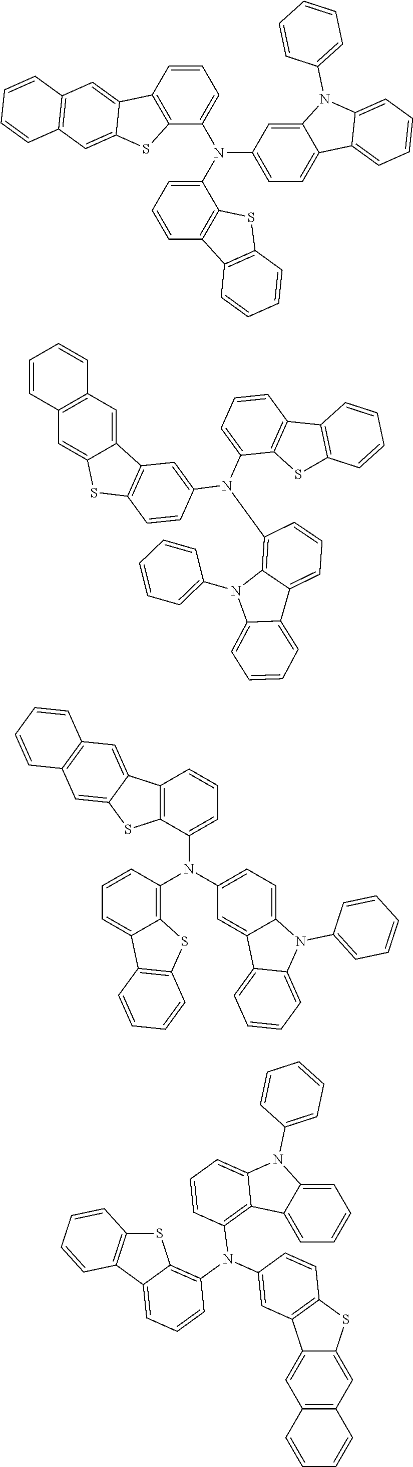 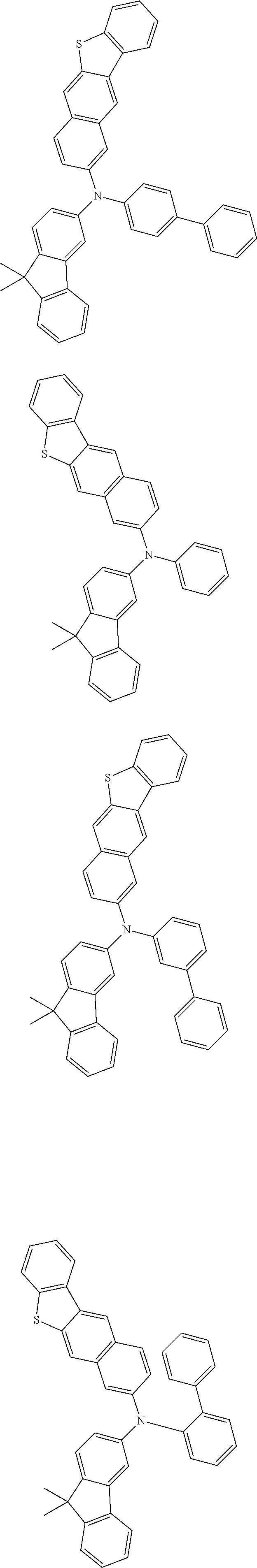 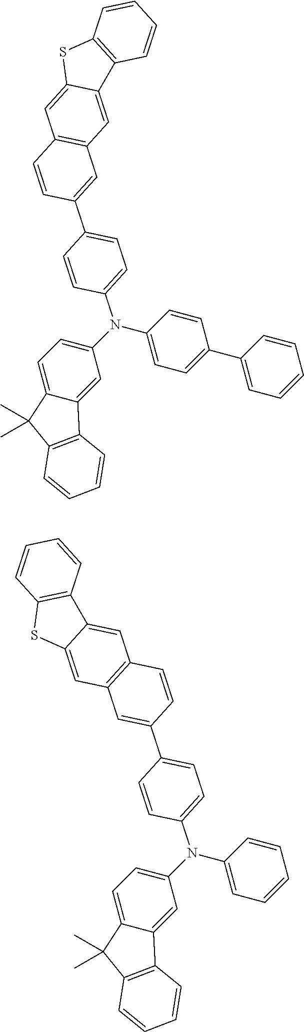 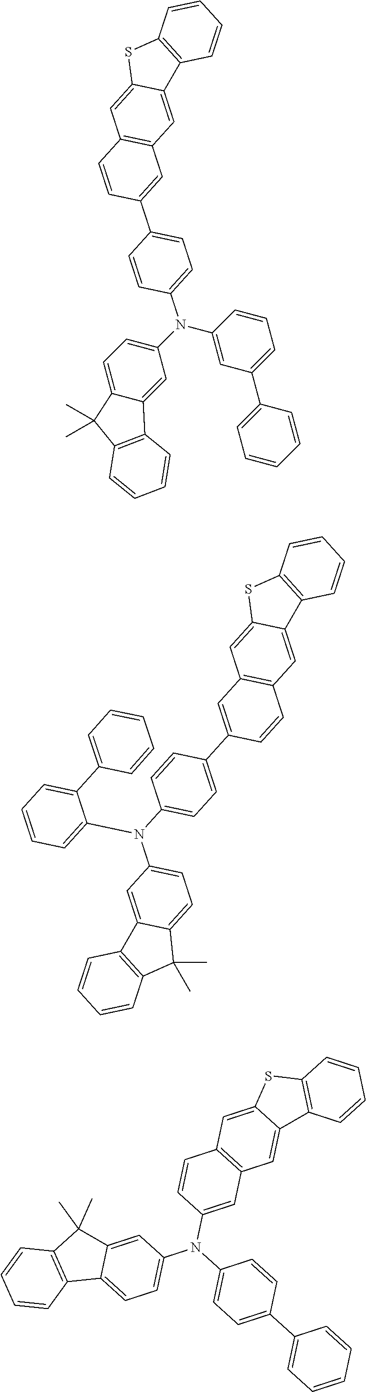 [SMILES: CC1(C)c2ccccc2-c2cc(N(c3ccc(-c4ccc5cc6sc7ccccc7c6cc5c4)cc3)c3cccc(-c4ccccc4)c3)ccc21 ; CC1(C)c2ccccc2-c2cc(N(c3ccc(-c4ccc5cc6c(cc5c4)sc4ccccc46)cc3)c3ccccc3-c3ccccc3)ccc21 ; CC1(C)c2ccccc2-c2ccc(N(c3ccc(-c4ccccc4)cc3)c3ccc4cc5sc6ccccc6c5cc4c3)cc21] 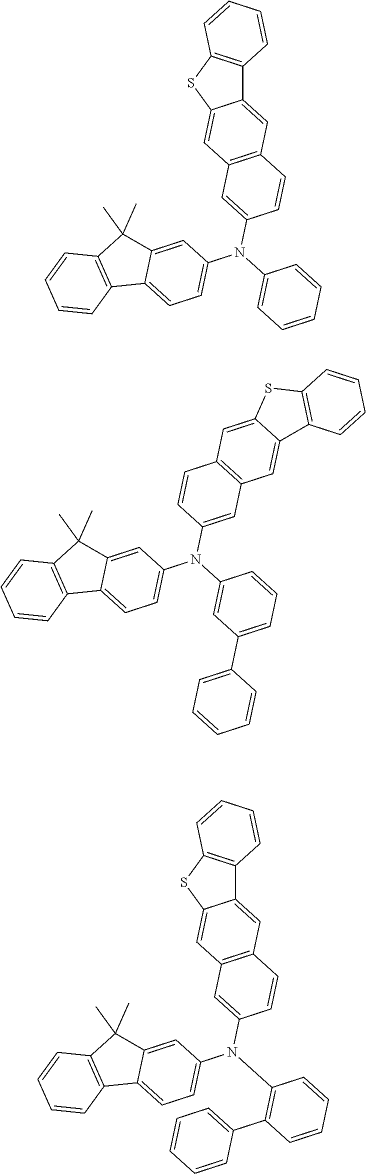 [SMILES: CC1(C)c2ccccc2-c2ccc(N(c3ccccc3)c3ccc4cc5c(cc4c3)sc3ccccc35)cc21 ; CC1(C)c2ccccc2-c2ccc(N(c3cccc(-c4ccccc4)c3)c3ccc4cc5sc6ccccc6c5cc4c3)cc21 ; CC1(C)c2ccccc2-c2ccc(N(c3ccc4cc5c(cc4c3)sc3ccccc35)c3ccccc3-c3ccccc3)cc21] 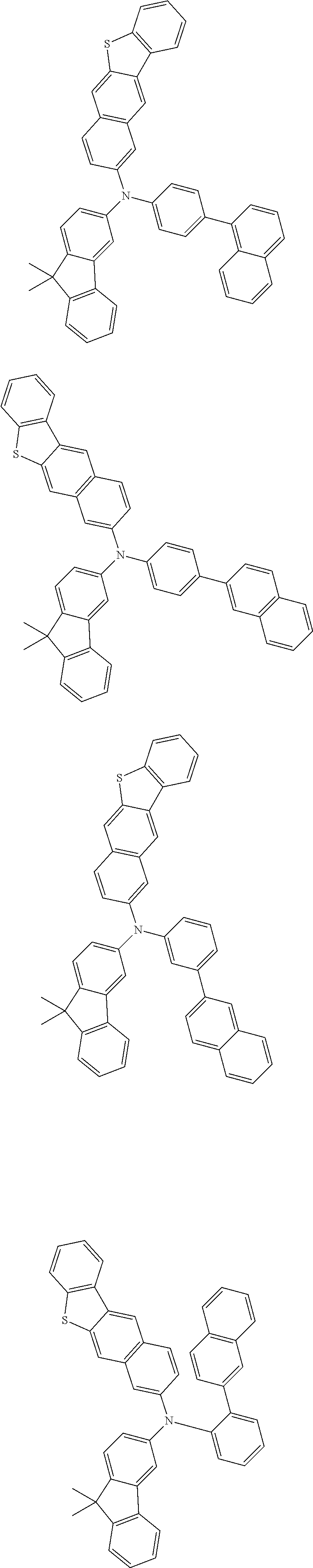 [SMILES: CC1(C)c2ccccc2-c2cc(N(c3ccc(-c4cccc5ccccc45)cc3)c3ccc4cc5sc6ccccc6c5cc4c3)ccc21 ; CC1(C)c2ccccc2-c2cc(N(c3ccc(-c4ccc5ccccc5c4)cc3)c3ccc4cc5c(cc4c3)sc3ccccc35)ccc21 ; CC1(C)c2ccccc2-c2cc(N(c3cccc(-c4ccc5ccccc5c4)c3)c3ccc4cc5sc6ccccc6c5cc4c3)ccc21 ; CC1(C)c2ccccc2-c2cc(N(c3ccc4cc5c(cc4c3)sc3ccccc35)c3ccccc3-c3ccc4ccccc4c3)ccc21] 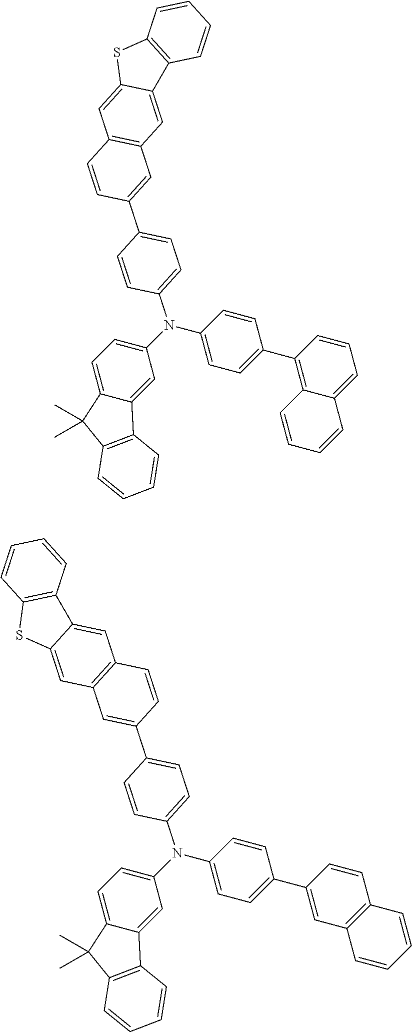 [SMILES: CC1(C)c2ccccc2-c2cc(N(c3ccc(-c4ccc5cc6sc7ccccc7c6cc5c4)cc3)c3ccc(-c4cccc5ccccc45)cc3)ccc21 ; CC1(C)c2ccccc2-c2cc(N(c3ccc(-c4ccc5ccccc5c4)cc3)c3ccc(-c4ccc5cc6c(cc5c4)sc4ccccc46)cc3)ccc21] 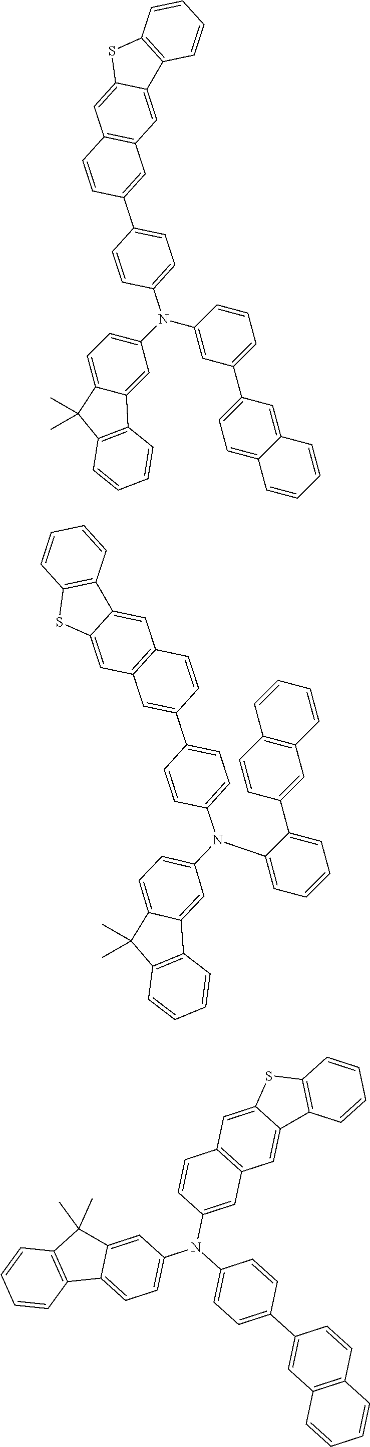 [SMILES: CC1(C)c2ccccc2-c2cc(N(c3ccc(-c4ccc5cc6sc7ccccc7c6cc5c4)cc3)c3cccc(-c4ccc5ccccc5c4)c3)ccc21 ; CC1(C)c2ccccc2-c2cc(N(c3ccc(-c4ccc5cc6c(cc5c4)sc4ccccc46)cc3)c3ccccc3-c3ccc4ccccc4c3)ccc21 ; CC1(C)c2ccccc2-c2ccc(N(c3ccc(-c4ccc5ccccc5c4)cc3)c3ccc4cc5sc6ccccc6c5cc4c3)cc21] 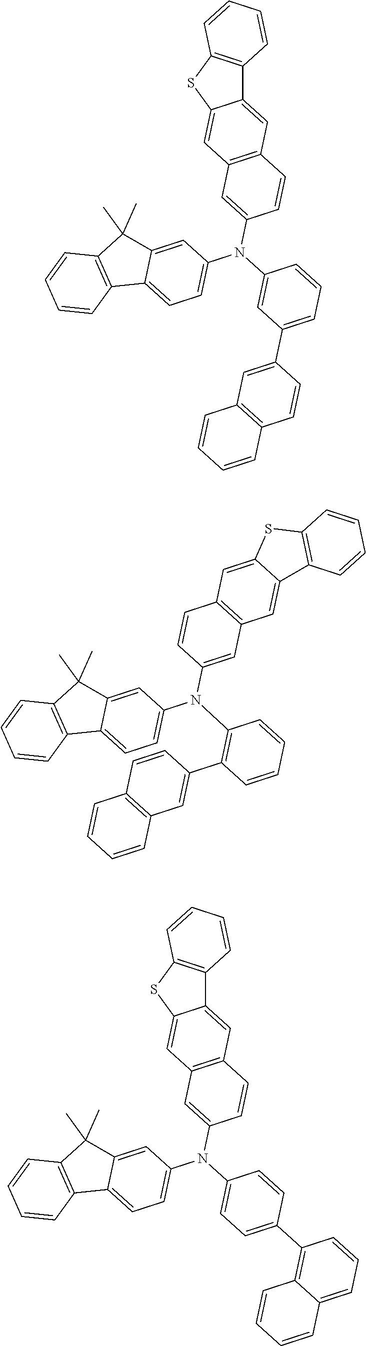 [SMILES: CC1(C)c2ccccc2-c2ccc(N(c3cccc(-c4ccc5ccccc5c4)c3)c3ccc4cc5c(cc4c3)sc3ccccc35)cc21 ; CC1(C)c2ccccc2-c2ccc(N(c3ccc4cc5sc6ccccc6c5cc4c3)c3ccccc3-c3ccc4ccccc4c3)cc21 ; CC1(C)c2ccccc2-c2ccc(N(c3ccc(-c4cccc5ccccc45)cc3)c3ccc4cc5c(cc4c3)sc3ccccc35)cc21] 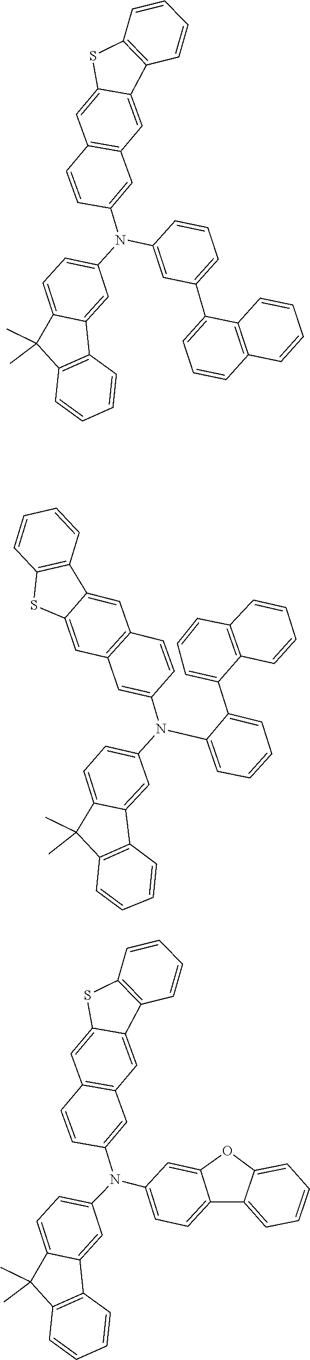 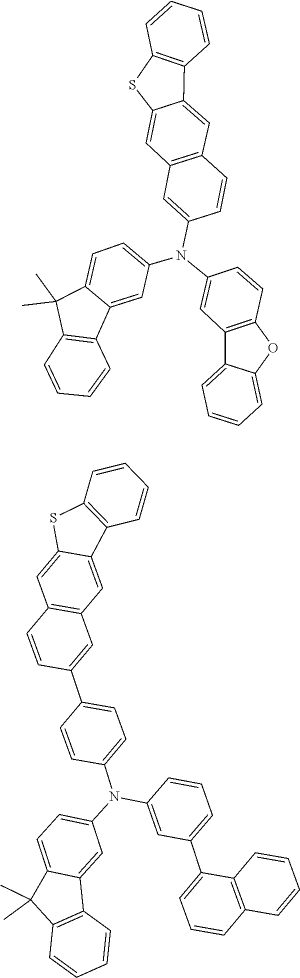 [SMILES: CC1(C)c2ccccc2-c2cc(N(c3ccc4cc5c(cc4c3)sc3ccccc35)c3ccc4oc5ccccc5c4c3)ccc21 ; CC1(C)c2ccccc2-c2cc(N(c3ccc(-c4ccc5cc6sc7ccccc7c6cc5c4)cc3)c3cccc(-c4cccc5ccccc45)c3)ccc21] 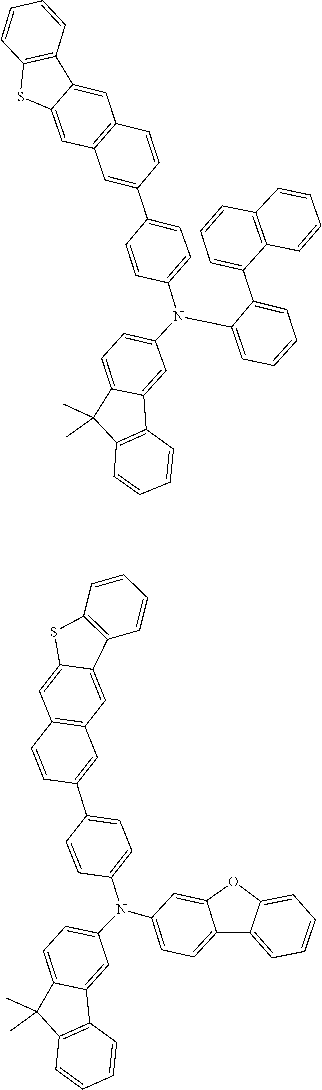 [SMILES: CC1(C)c2ccccc2-c2cc(N(c3ccc(-c4ccc5cc6c(cc5c4)sc4ccccc46)cc3)c3ccccc3-c3cccc4ccccc34)ccc21 ; CC1(C)c2ccccc2-c2cc(N(c3ccc(-c4ccc5cc6sc7ccccc7c6cc5c4)cc3)c3ccc4c(c3)oc3ccccc34)ccc21] 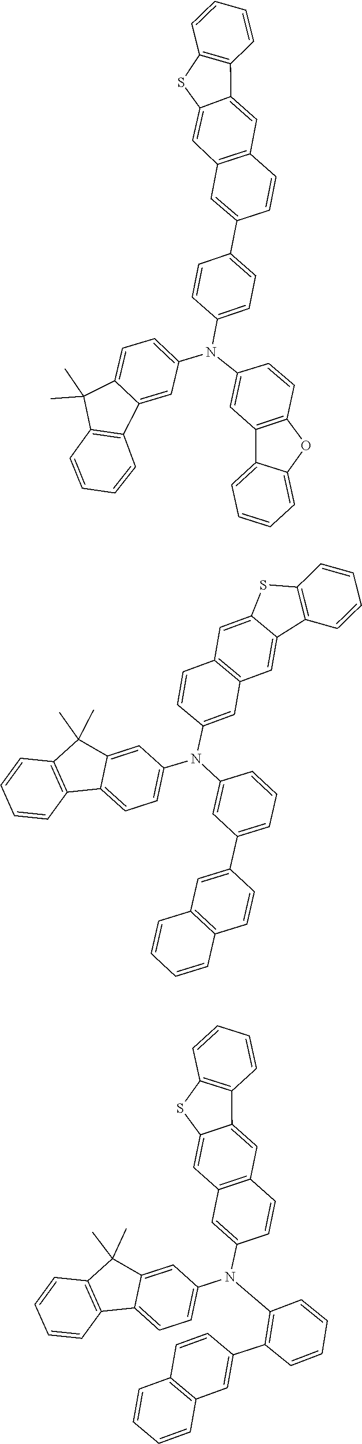 [SMILES: CC1(C)c2ccccc2-c2cc(N(c3ccc(-c4ccc5cc6c(cc5c4)sc4ccccc46)cc3)c3ccc4oc5ccccc5c4c3)ccc21 ; CC1(C)c2ccccc2-c2ccc(N(c3cccc(-c4ccc5ccccc5c4)c3)c3ccc4cc5sc6ccccc6c5cc4c3)cc21 ; CC1(C)c2ccccc2-c2ccc(N(c3ccc4cc5c(cc4c3)sc3ccccc35)c3ccccc3-c3ccc4ccccc4c3)cc21] 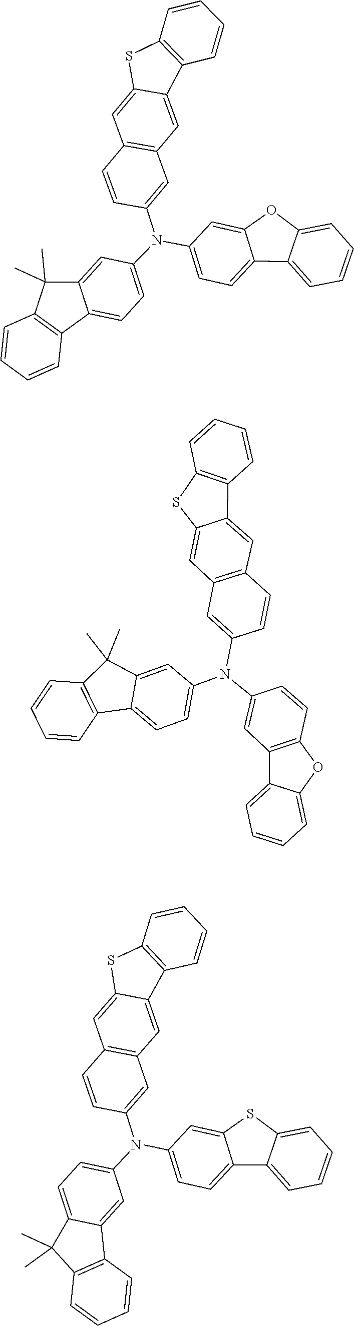 [SMILES: CC1(C)c2ccccc2-c2ccc(N(c3ccc4cc5sc6ccccc6c5cc4c3)c3ccc4c(c3)oc3ccccc34)cc21 ; CC1(C)c2ccccc2-c2ccc(N(c3ccc4cc5c(cc4c3)sc3ccccc35)c3ccc4oc5ccccc5c4c3)cc21 ; CC1(C)c2ccccc2-c2cc(N(c3ccc4cc5sc6ccccc6c5cc4c3)c3ccc4c(c3)sc3ccccc34)ccc21] 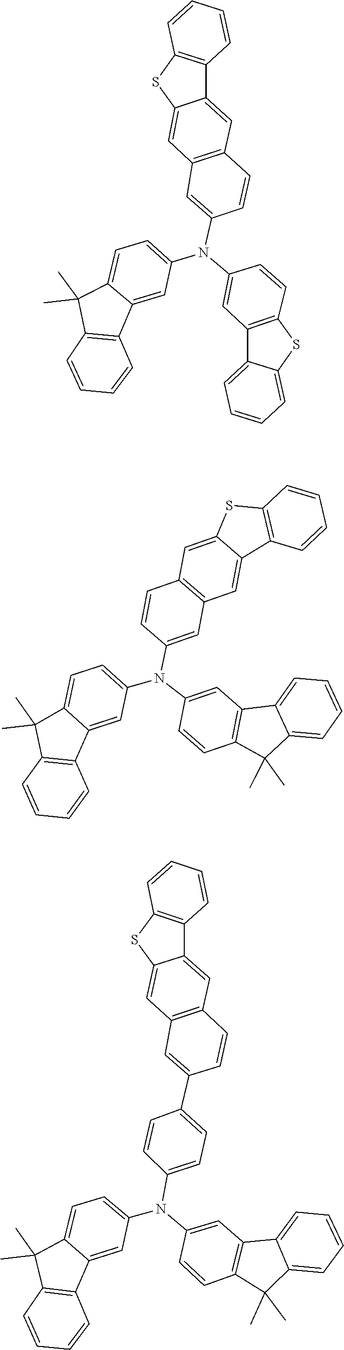 [SMILES: CC1(C)c2ccccc2-c2cc(N(c3ccc4cc5c(cc4c3)sc3ccccc35)c3ccc4sc5ccccc5c4c3)ccc21 ; CC1(C)c2ccccc2-c2cc(N(c3ccc4c(c3)-c3ccccc3C4(C)C)c3ccc4cc5sc6ccccc6c5cc4c3)ccc21 ; CC1(C)c2ccccc2-c2cc(N(c3ccc(-c4ccc5cc6c(cc5c4)sc4ccccc46)cc3)c3ccc4c(c3)-c3ccccc3C4(C)C)ccc21] 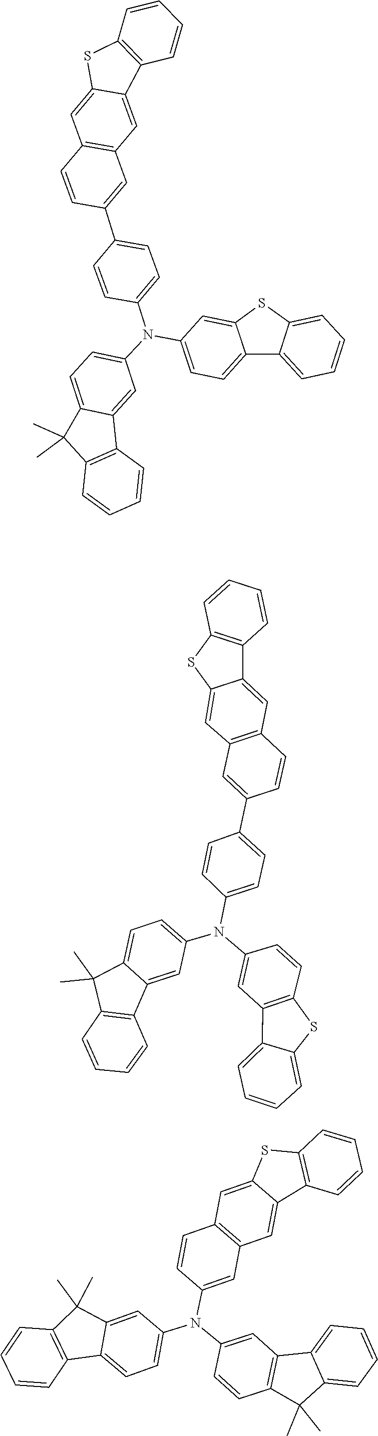 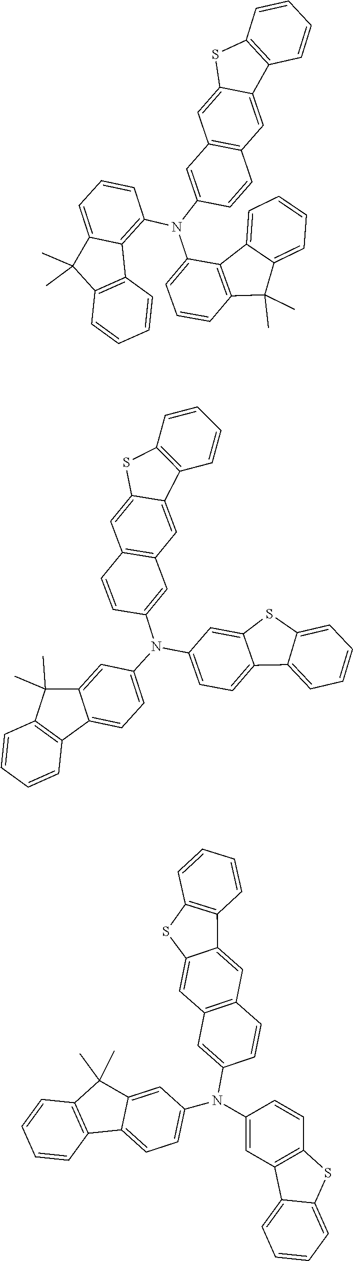 [SMILES: CC1(C)c2ccccc2-c2c(N(c3ccc4cc5c(cc4c3)sc3ccccc35)c3cccc4c3-c3ccccc3C4(C)C)cccc21 ; CC1(C)c2ccccc2-c2ccc(N(c3ccc4cc5sc6ccccc6c5cc4c3)c3ccc4c(c3)sc3ccccc34)cc21 ; CC1(C)c2ccccc2-c2ccc(N(c3ccc4cc5c(cc4c3)sc3ccccc35)c3ccc4sc5ccccc5c4c3)cc21] 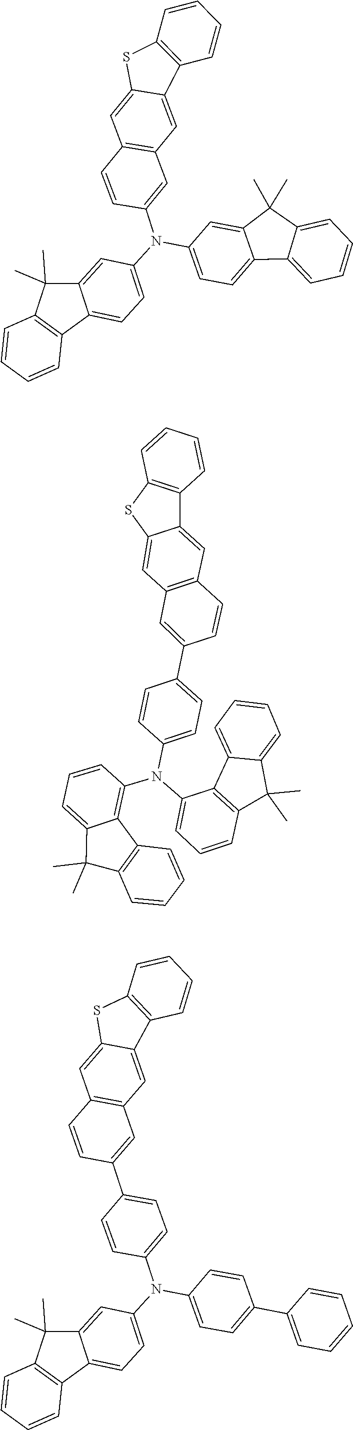 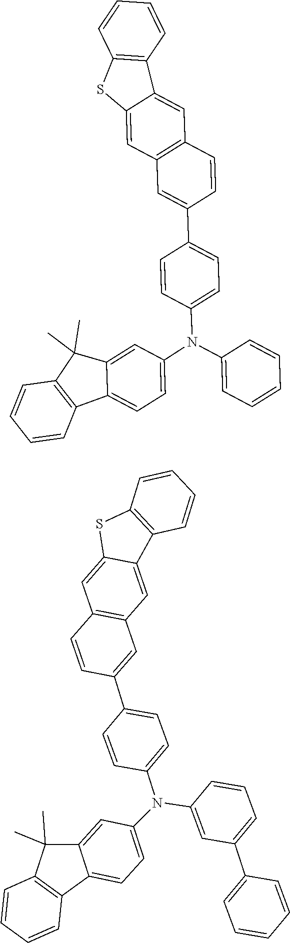 [SMILES: CC1(C)c2ccccc2-c2ccc(N(c3ccccc3)c3ccc(-c4ccc5cc6c(cc5c4)sc4ccccc46)cc3)cc21 ; CC1(C)c2ccccc2-c2ccc(N(c3ccc(-c4ccc5cc6sc7ccccc7c6cc5c4)cc3)c3cccc(-c4ccccc4)c3)cc21] 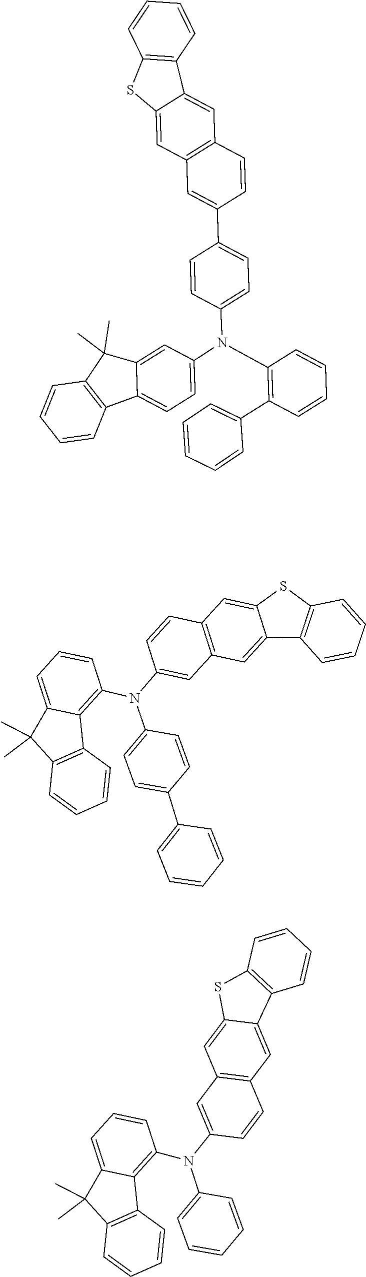 [SMILES: CC1(C)c2ccccc2-c2ccc(N(c3ccc(-c4ccc5cc6c(cc5c4)sc4ccccc46)cc3)c3ccccc3-c3ccccc3)cc21 ; CC1(C)c2ccccc2-c2c(N(c3ccc(-c4ccccc4)cc3)c3ccc4cc5sc6ccccc6c5cc4c3)cccc21 ; CC1(C)c2ccccc2-c2c(N(c3ccccc3)c3ccc4cc5c(cc4c3)sc3ccccc35)cccc21] 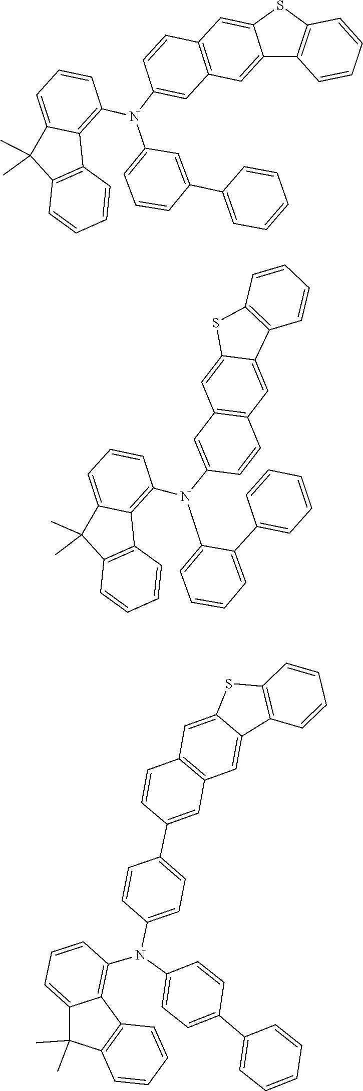 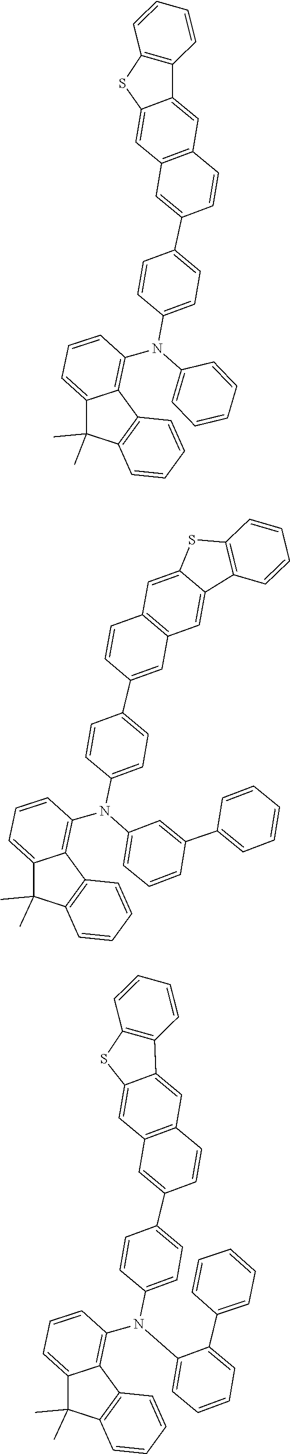 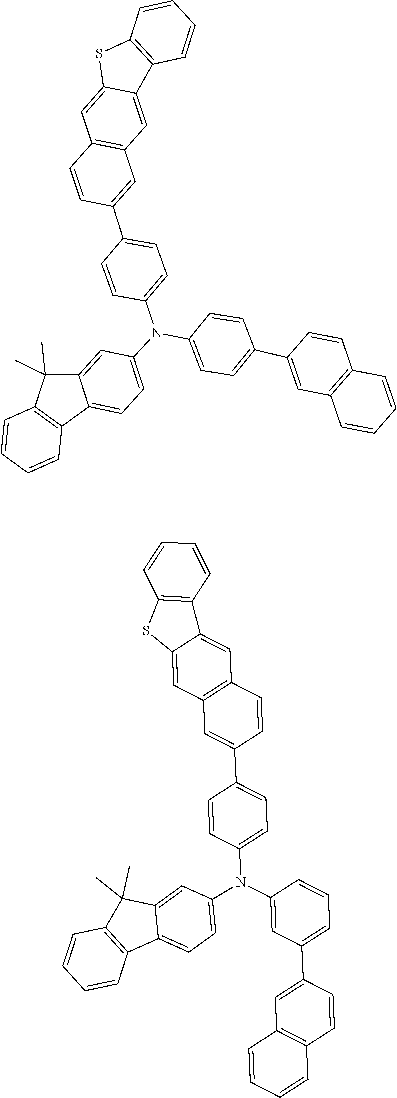 [SMILES: CC1(C)c2ccccc2-c2ccc(N(c3ccc(-c4ccc5ccccc5c4)cc3)c3ccc(-c4ccc5cc6sc7ccccc7c6cc5c4)cc3)cc21 ; CC1(C)c2ccccc2-c2ccc(N(c3ccc(-c4ccc5cc6c(cc5c4)sc4ccccc46)cc3)c3cccc(-c4ccc5ccccc5c4)c3)cc21] 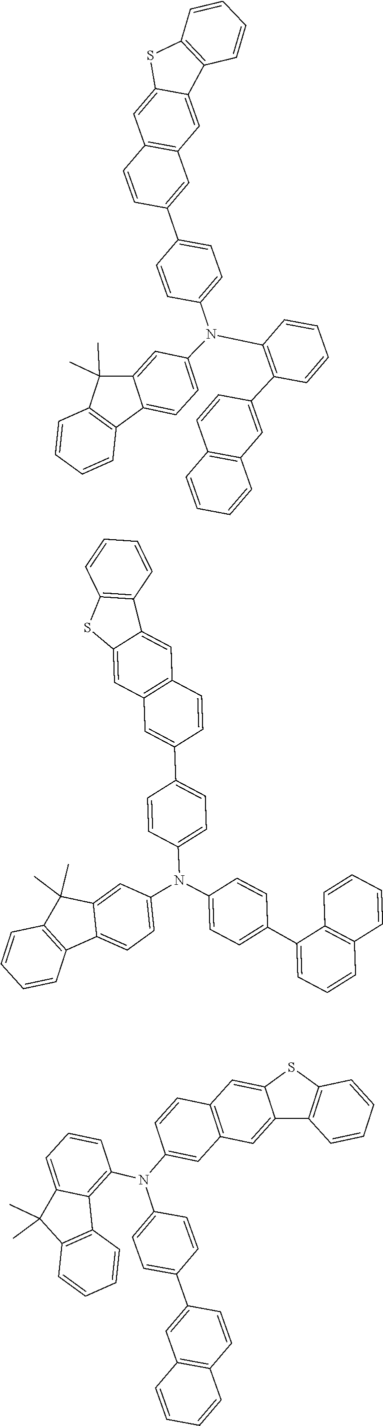 [SMILES: CC1(C)c2ccccc2-c2ccc(N(c3ccc(-c4ccc5cc6sc7ccccc7c6cc5c4)cc3)c3ccccc3-c3ccc4ccccc4c3)cc21 ; CC1(C)c2ccccc2-c2ccc(N(c3ccc(-c4ccc5cc6c(cc5c4)sc4ccccc46)cc3)c3ccc(-c4cccc5ccccc45)cc3)cc21 ; CC1(C)c2ccccc2-c2c(N(c3ccc(-c4ccc5ccccc5c4)cc3)c3ccc4cc5sc6ccccc6c5cc4c3)cccc21] 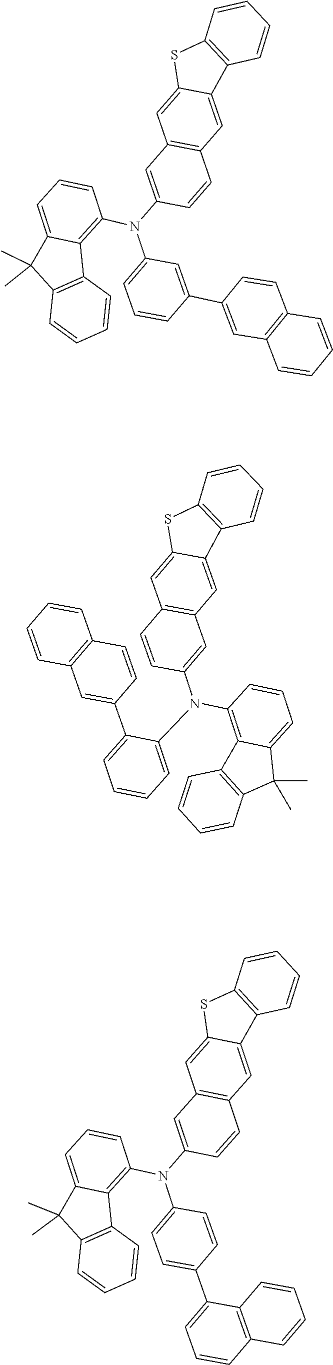 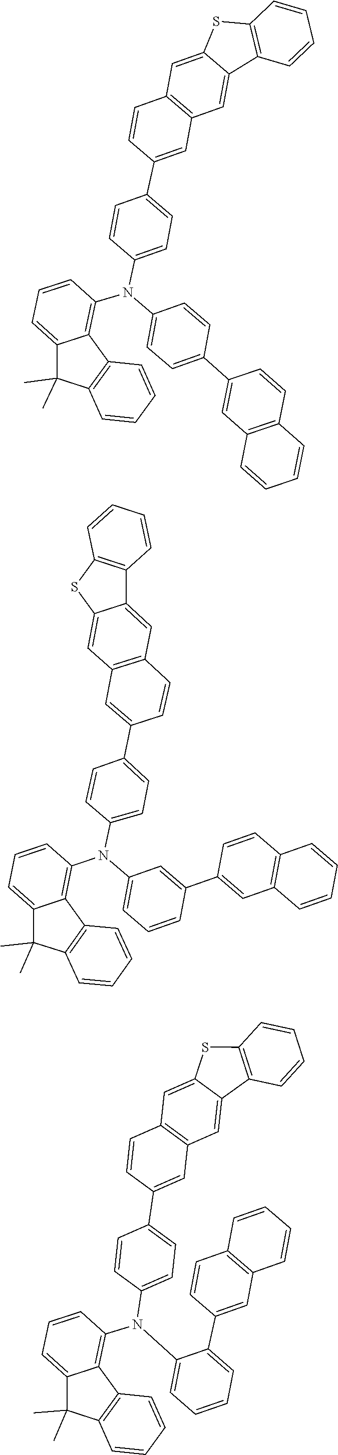 [SMILES: CC1(C)c2ccccc2-c2c(N(c3ccc(-c4ccc5ccccc5c4)cc3)c3ccc(-c4ccc5cc6sc7ccccc7c6cc5c4)cc3)cccc21 ; CC1(C)c2ccccc2-c2c(N(c3ccc(-c4ccc5cc6c(cc5c4)sc4ccccc46)cc3)c3cccc(-c4ccc5ccccc5c4)c3)cccc21 ; CC1(C)c2ccccc2-c2c(N(c3ccc(-c4ccc5cc6sc7ccccc7c6cc5c4)cc3)c3ccccc3-c3ccc4ccccc4c3)cccc21] 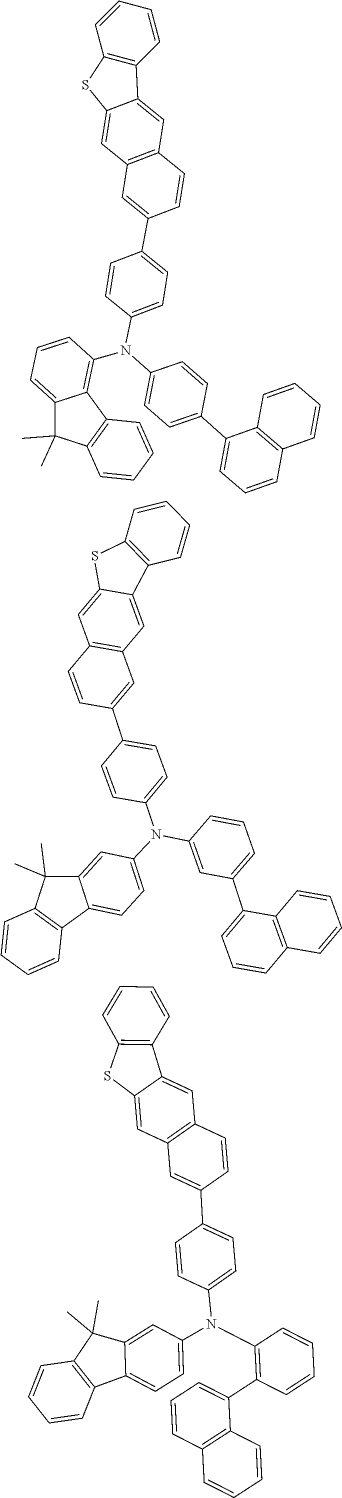 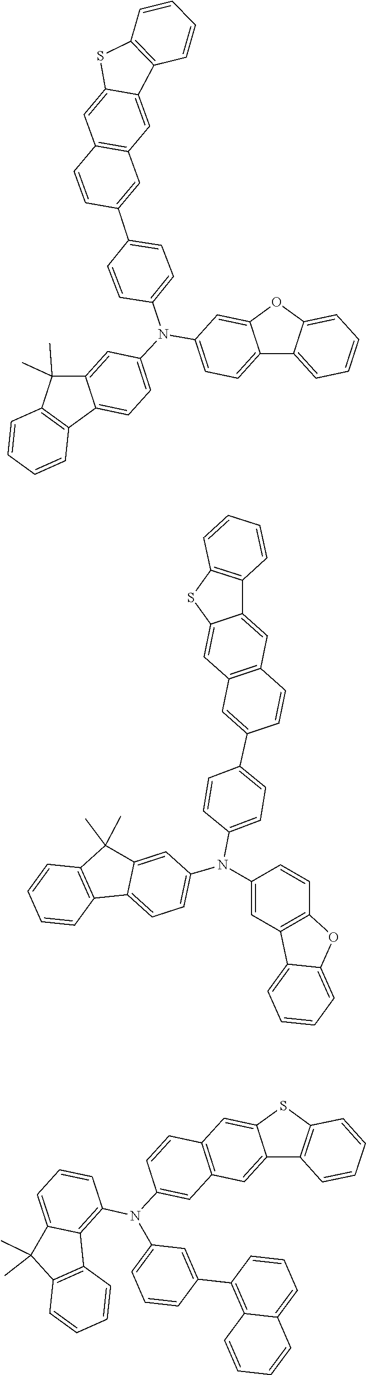 [SMILES: CC1(C)c2ccccc2-c2ccc(N(c3ccc(-c4ccc5cc6sc7ccccc7c6cc5c4)cc3)c3ccc4c(c3)oc3ccccc34)cc21 ; CC1(C)c2ccccc2-c2ccc(N(c3ccc(-c4ccc5cc6c(cc5c4)sc4ccccc46)cc3)c3ccc4oc5ccccc5c4c3)cc21 ; CC1(C)c2ccccc2-c2c(N(c3cccc(-c4cccc5ccccc45)c3)c3ccc4cc5sc6ccccc6c5cc4c3)cccc21] 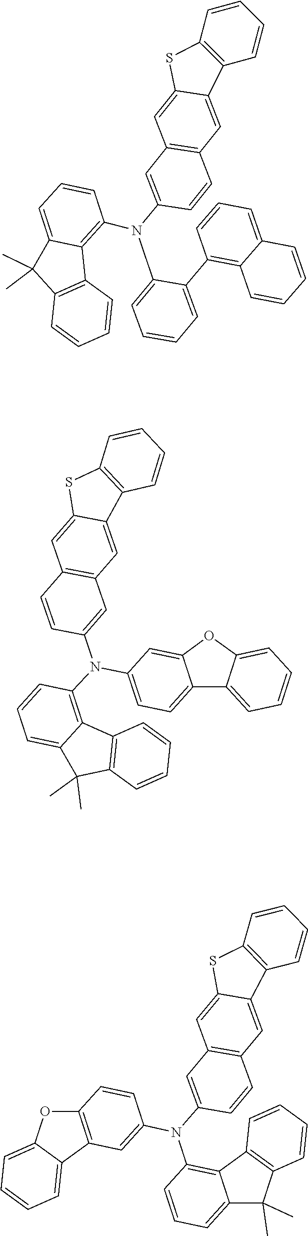 [SMILES: CC1(C)c2ccccc2-c2c(N(c3ccc4cc5c(cc4c3)sc3ccccc35)c3ccccc3-c3cccc4ccccc34)cccc21 ; CC1(C)c2ccccc2-c2c(N(c3ccc4cc5sc6ccccc6c5cc4c3)c3ccc4c(c3)oc3ccccc34)cccc21 ; CC1(C)c2ccccc2-c2c(N(c3ccc4cc5c(cc4c3)sc3ccccc35)c3ccc4oc5ccccc5c4c3)cccc21] 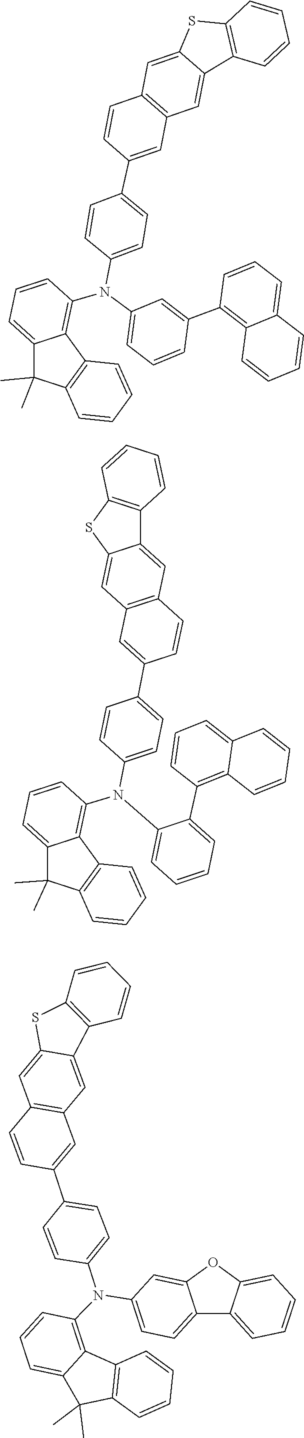 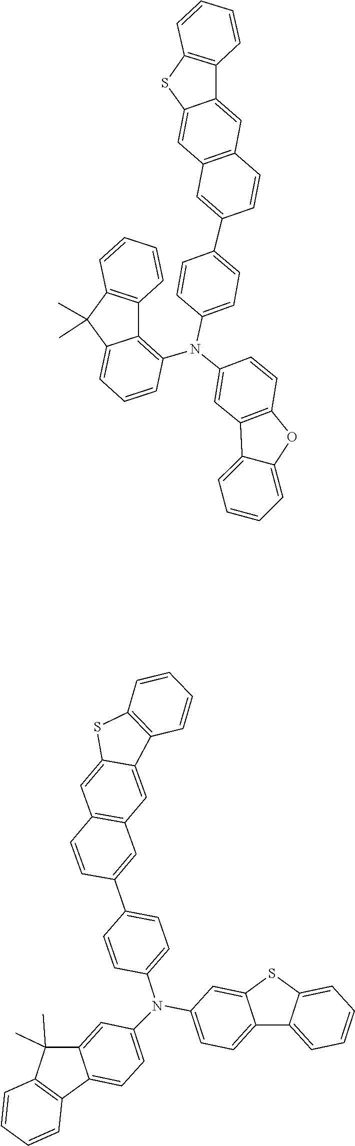 [SMILES: CC1(C)c2ccccc2-c2c(N(c3ccc(-c4ccc5cc6c(cc5c4)sc4ccccc46)cc3)c3ccc4oc5ccccc5c4c3)cccc21 ; CC1(C)c2ccccc2-c2ccc(N(c3ccc(-c4ccc5cc6sc7ccccc7c6cc5c4)cc3)c3ccc4c(c3)sc3ccccc34)cc21] 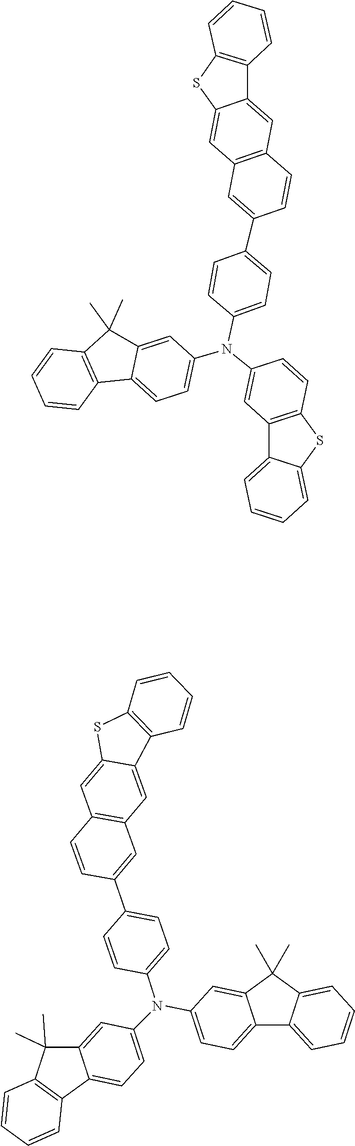 [SMILES: CC1(C)c2ccccc2-c2ccc(N(c3ccc(-c4ccc5cc6c(cc5c4)sc4ccccc46)cc3)c3ccc4sc5ccccc5c4c3)cc21 ; CC1(C)c2ccccc2-c2ccc(N(c3ccc(-c4ccc5cc6sc7ccccc7c6cc5c4)cc3)c3ccc4c(c3)C(C)(C)c3ccccc3-4)cc21] 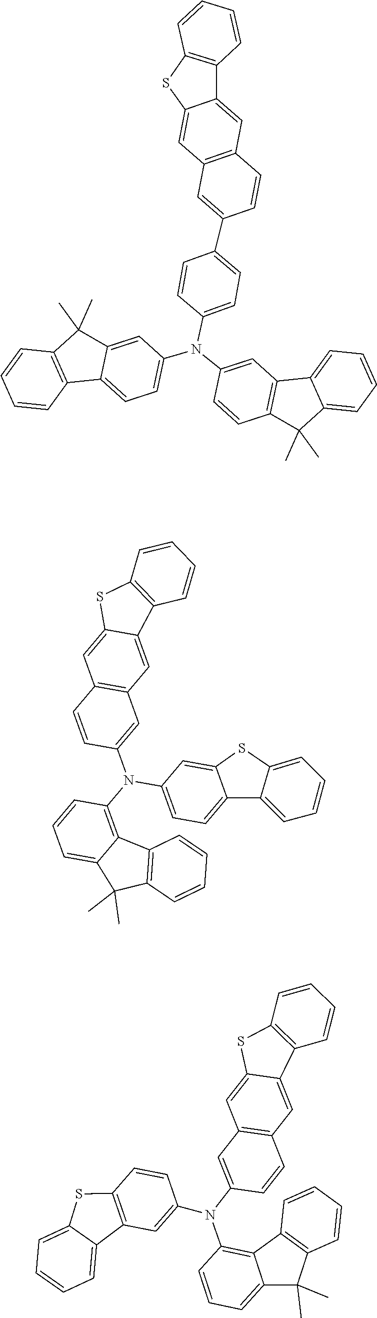 [SMILES: CC1(C)c2ccccc2-c2cc(N(c3ccc(-c4ccc5cc6c(cc5c4)sc4ccccc46)cc3)c3ccc4c(c3)C(C)(C)c3ccccc3-4)ccc21 ; CC1(C)c2ccccc2-c2c(N(c3ccc4cc5sc6ccccc6c5cc4c3)c3ccc4c(c3)sc3ccccc34)cccc21 ; CC1(C)c2ccccc2-c2c(N(c3ccc4cc5c(cc4c3)sc3ccccc35)c3ccc4sc5ccccc5c4c3)cccc21] 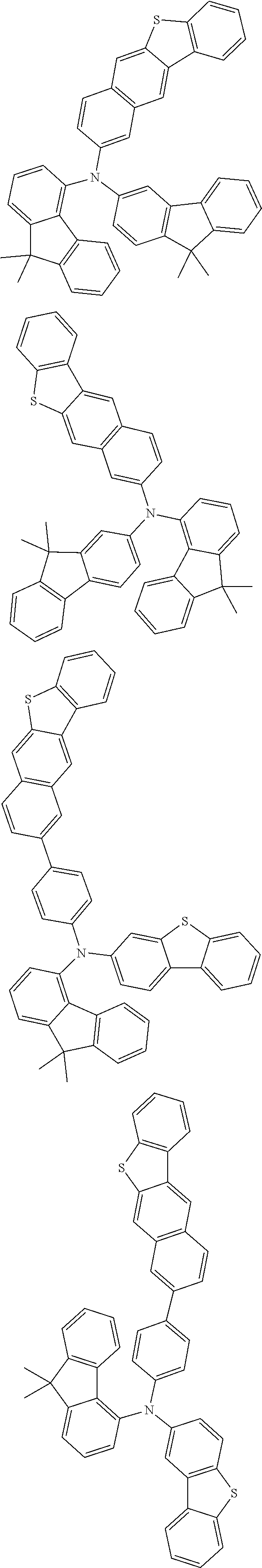 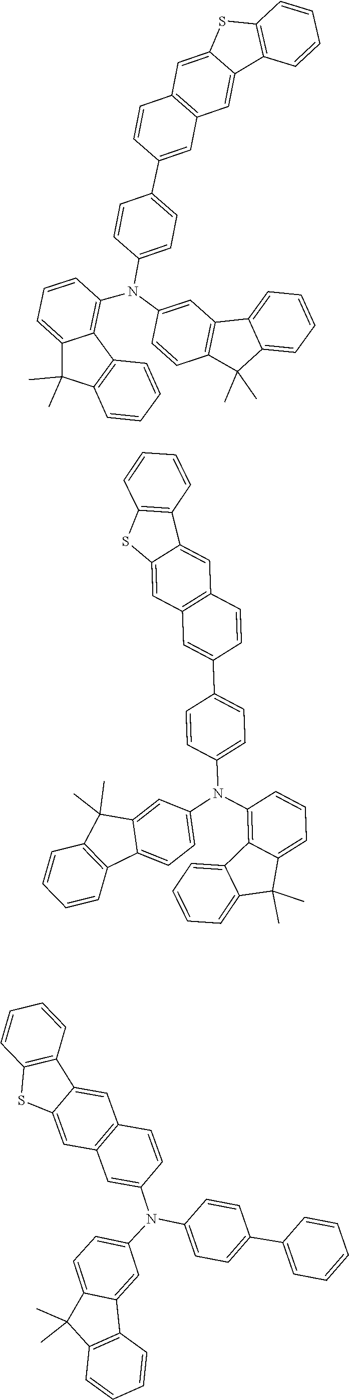 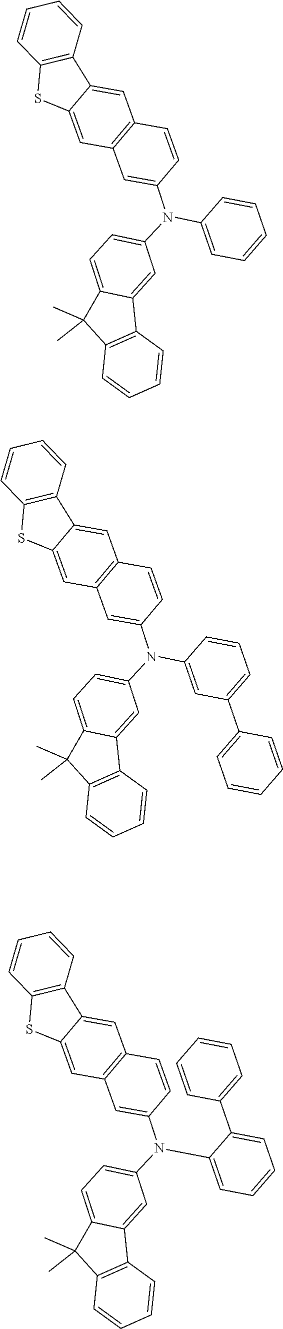 [SMILES: CC1(C)c2ccccc2-c2cc(N(c3ccccc3)c3ccc4cc5c(cc4c3)sc3ccccc35)ccc21 ; CC1(C)c2ccccc2-c2cc(N(c3cccc(-c4ccccc4)c3)c3ccc4cc5c(cc4c3)sc3ccccc35)ccc21 ; CC1(C)c2ccccc2-c2cc(N(c3ccc4cc5c(cc4c3)sc3ccccc35)c3ccccc3-c3ccccc3)ccc21] 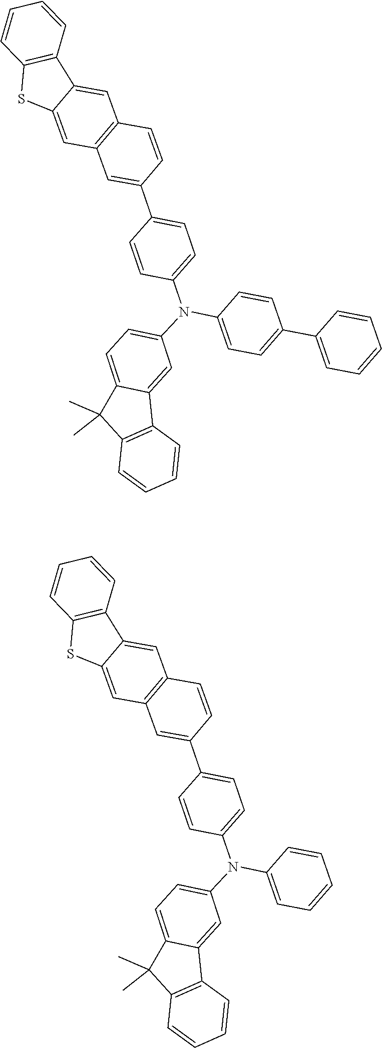 [SMILES: CC1(C)c2ccccc2-c2cc(N(c3ccc(-c4ccccc4)cc3)c3ccc(-c4ccc5cc6c(cc5c4)sc4ccccc46)cc3)ccc21 ; CC1(C)c2ccccc2-c2cc(N(c3ccccc3)c3ccc(-c4ccc5cc6c(cc5c4)sc4ccccc46)cc3)ccc21] 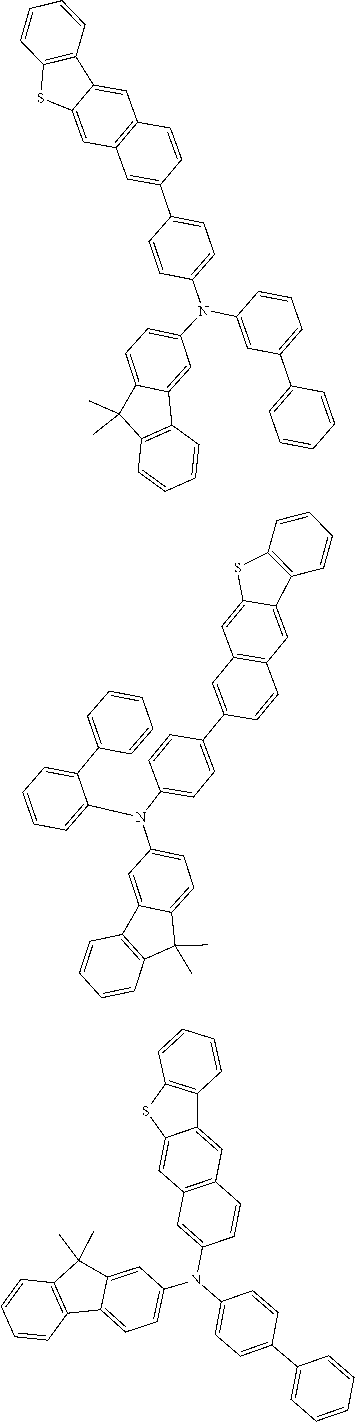 [SMILES: CC1(C)c2ccccc2-c2cc(N(c3ccc(-c4ccc5cc6c(cc5c4)sc4ccccc46)cc3)c3cccc(-c4ccccc4)c3)ccc21 ; CC1(C)c2ccccc2-c2cc(N(c3ccc(-c4ccc5cc6c(cc5c4)sc4ccccc46)cc3)c3ccccc3-c3ccccc3)ccc21 ; CC1(C)c2ccccc2-c2ccc(N(c3ccc(-c4ccccc4)cc3)c3ccc4cc5c(cc4c3)sc3ccccc35)cc21] 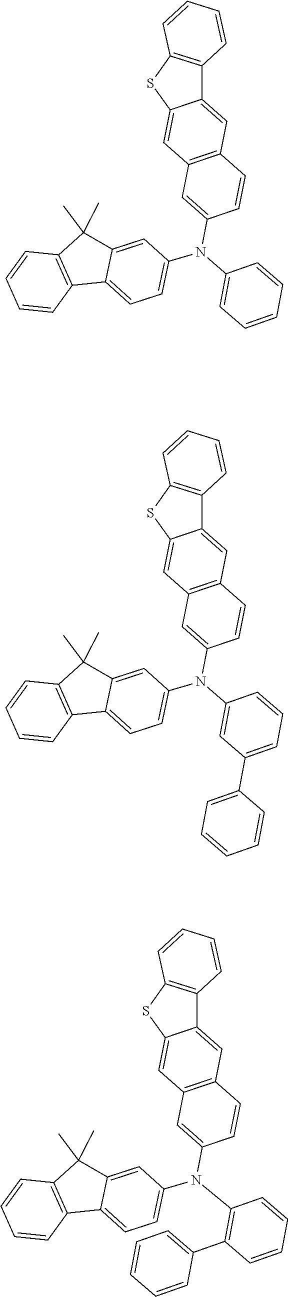 [SMILES: CC1(C)c2ccccc2-c2ccc(N(c3ccccc3)c3ccc4cc5c(cc4c3)sc3ccccc35)cc21 ; CC1(C)c2ccccc2-c2ccc(N(c3cccc(-c4ccccc4)c3)c3ccc4cc5c(cc4c3)sc3ccccc35)cc21 ; CC1(C)c2ccccc2-c2ccc(N(c3ccc4cc5c(cc4c3)sc3ccccc35)c3ccccc3-c3ccccc3)cc21] 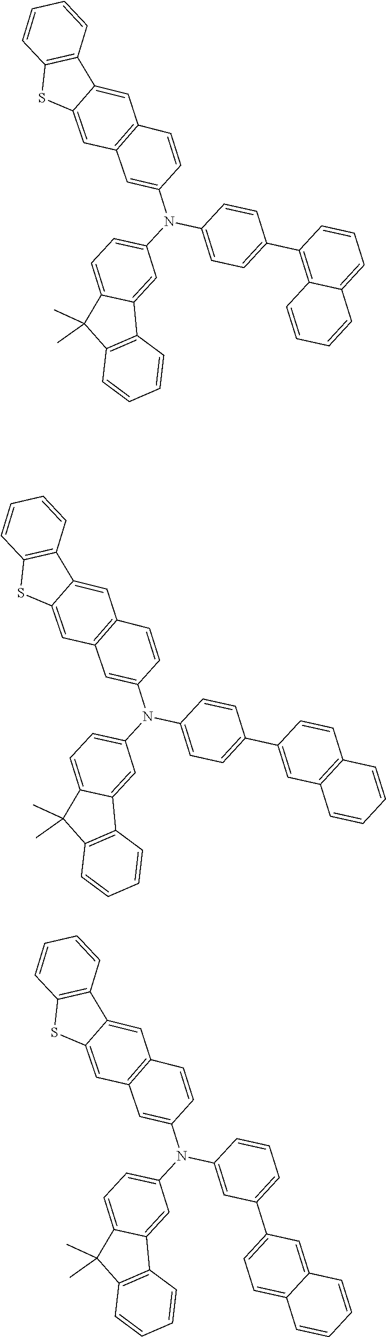 [SMILES: CC1(C)c2ccccc2-c2cc(N(c3ccc(-c4cccc5ccccc45)cc3)c3ccc4cc5c(cc4c3)sc3ccccc35)ccc21 ; CC1(C)c2ccccc2-c2cc(N(c3ccc(-c4ccc5ccccc5c4)cc3)c3ccc4cc5c(cc4c3)sc3ccccc35)ccc21 ; CC1(C)c2ccccc2-c2cc(N(c3cccc(-c4ccc5ccccc5c4)c3)c3ccc4cc5c(cc4c3)sc3ccccc35)ccc21] 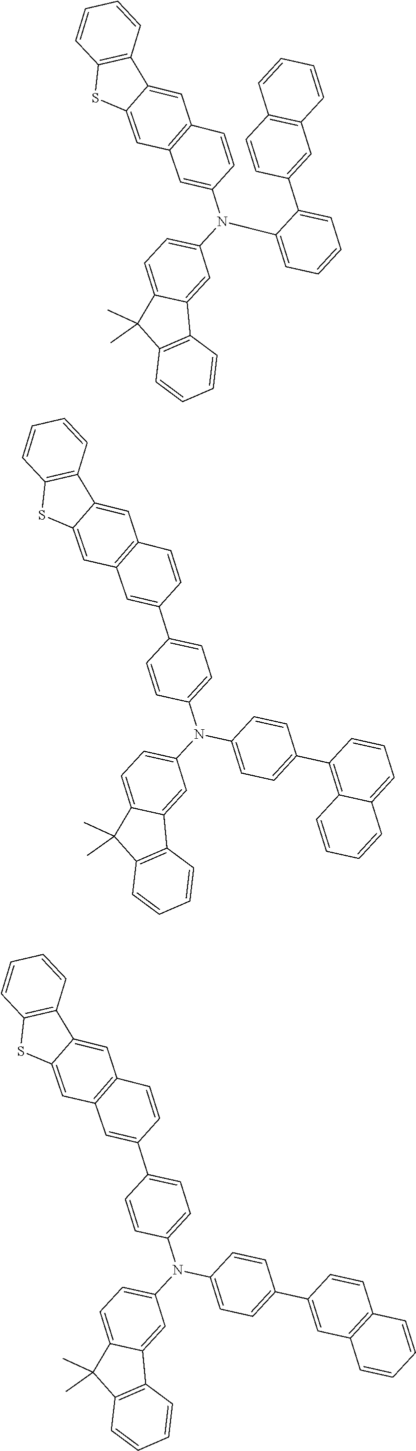 [SMILES: CC1(C)c2ccccc2-c2cc(N(c3ccc4cc5c(cc4c3)sc3ccccc35)c3ccccc3-c3ccc4ccccc4c3)ccc21 ; CC1(C)c2ccccc2-c2cc(N(c3ccc(-c4ccc5cc6c(cc5c4)sc4ccccc46)cc3)c3ccc(-c4cccc5ccccc45)cc3)ccc21 ; CC1(C)c2ccccc2-c2cc(N(c3ccc(-c4ccc5ccccc5c4)cc3)c3ccc(-c4ccc5cc6c(cc5c4)sc4ccccc46)cc3)ccc21] 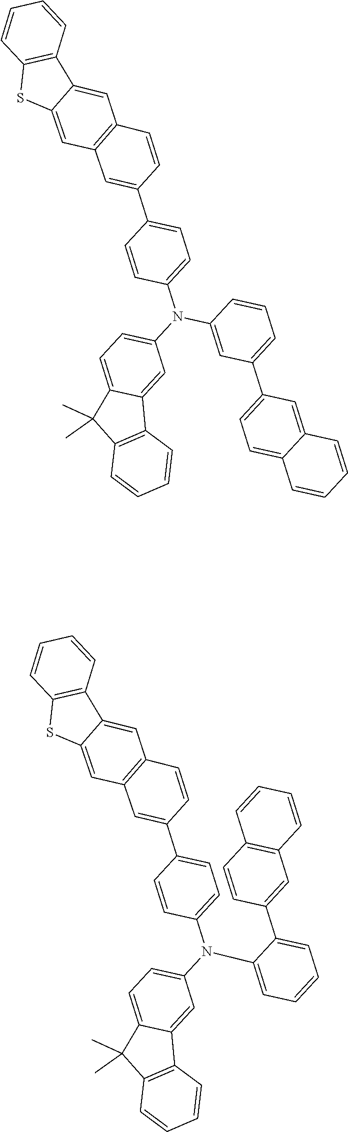 [SMILES: CC1(C)c2ccccc2-c2cc(N(c3ccc(-c4ccc5cc6c(cc5c4)sc4ccccc46)cc3)c3cccc(-c4ccc5ccccc5c4)c3)ccc21 ; CC1(C)c2ccccc2-c2cc(N(c3ccc(-c4ccc5cc6c(cc5c4)sc4ccccc46)cc3)c3ccccc3-c3ccc4ccccc4c3)ccc21] 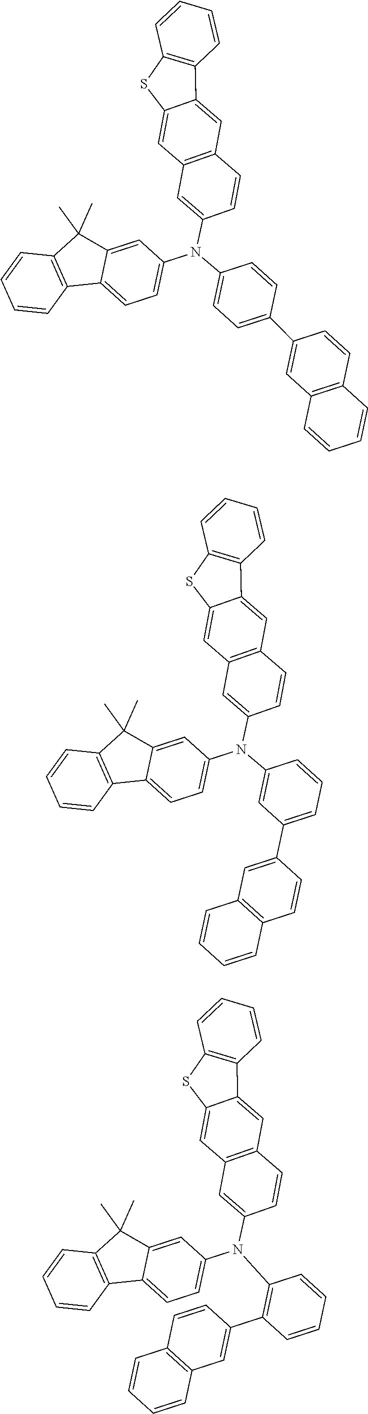 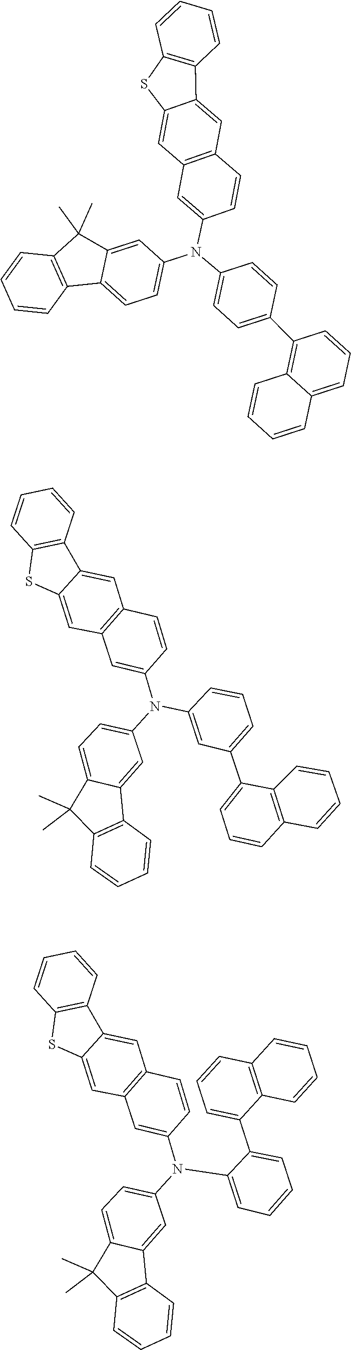 [SMILES: CC1(C)c2ccccc2-c2ccc(N(c3ccc(-c4cccc5ccccc45)cc3)c3ccc4cc5c(cc4c3)sc3ccccc35)cc21 ; CC1(C)c2ccccc2-c2cc(N(c3cccc(-c4cccc5ccccc45)c3)c3ccc4cc5c(cc4c3)sc3ccccc35)ccc21 ; CC1(C)c2ccccc2-c2cc(N(c3ccc4cc5c(cc4c3)sc3ccccc35)c3ccccc3-c3cccc4ccccc34)ccc21] 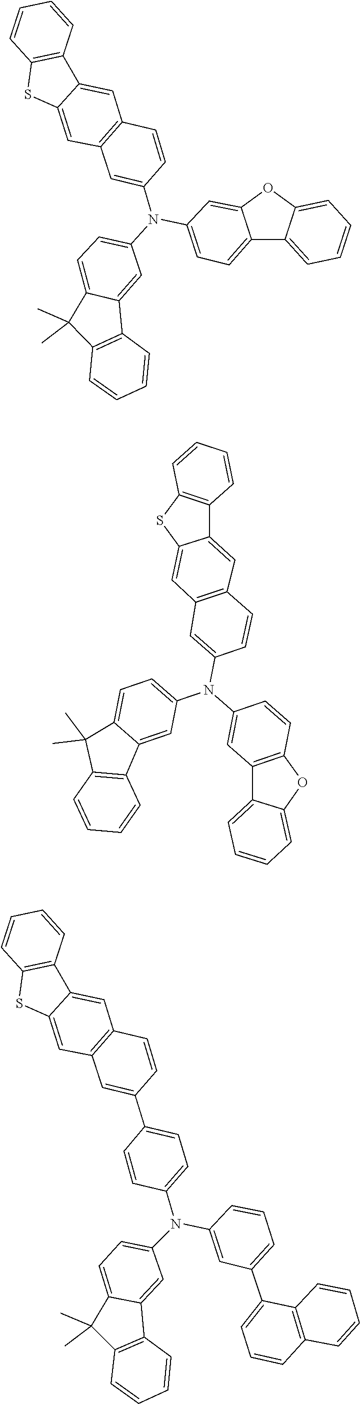 [SMILES: CC1(C)c2ccccc2-c2cc(N(c3ccc4cc5c(cc4c3)sc3ccccc35)c3ccc4c(c3)oc3ccccc34)ccc21 ; CC1(C)c2ccccc2-c2cc(N(c3ccc4cc5c(cc4c3)sc3ccccc35)c3ccc4oc5ccccc5c4c3)ccc21 ; CC1(C)c2ccccc2-c2cc(N(c3ccc(-c4ccc5cc6c(cc5c4)sc4ccccc46)cc3)c3cccc(-c4cccc5ccccc45)c3)ccc21] 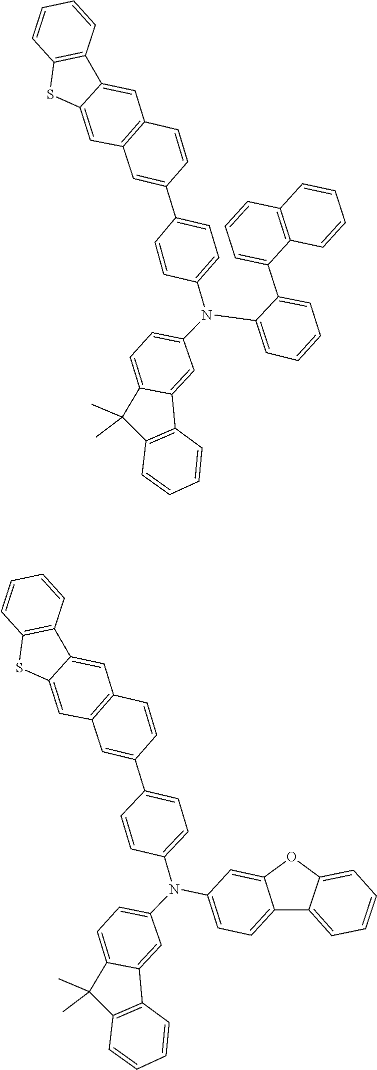 [SMILES: CC1(C)c2ccccc2-c2cc(N(c3ccc(-c4ccc5cc6c(cc5c4)sc4ccccc46)cc3)c3ccccc3-c3cccc4ccccc34)ccc21 ; CC1(C)c2ccccc2-c2cc(N(c3ccc(-c4ccc5cc6c(cc5c4)sc4ccccc46)cc3)c3ccc4c(c3)oc3ccccc34)ccc21] 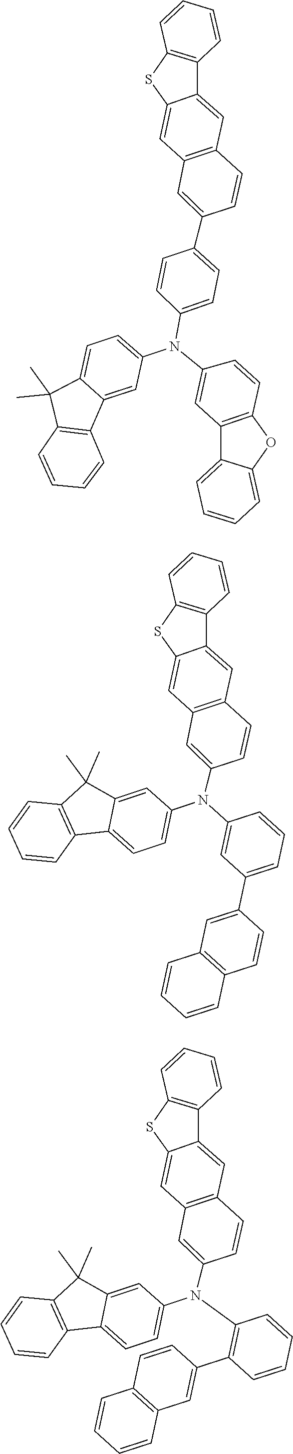 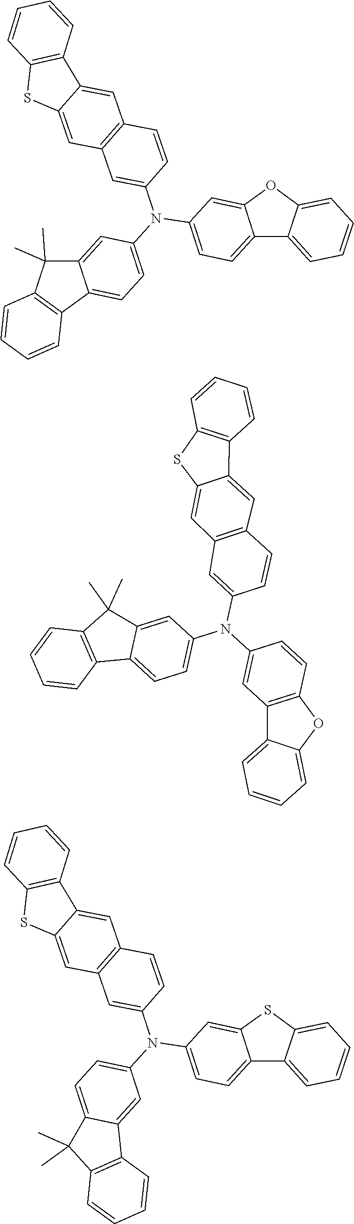 [SMILES: CC1(C)c2ccccc2-c2ccc(N(c3ccc4cc5c(cc4c3)sc3ccccc35)c3ccc4c(c3)oc3ccccc34)cc21 ; CC1(C)c2ccccc2-c2ccc(N(c3ccc4cc5c(cc4c3)sc3ccccc35)c3ccc4oc5ccccc5c4c3)cc21 ; CC1(C)c2ccccc2-c2cc(N(c3ccc4cc5c(cc4c3)sc3ccccc35)c3ccc4c(c3)sc3ccccc34)ccc21] 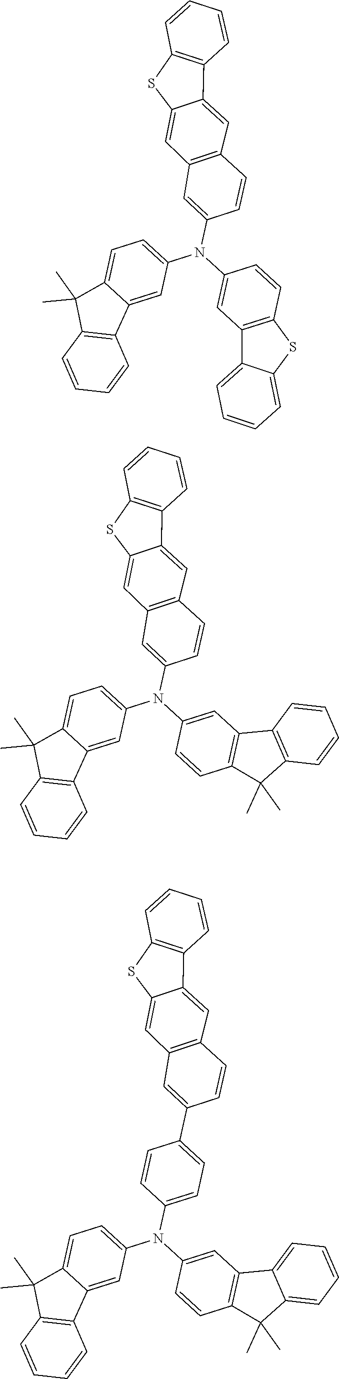 [SMILES: CC1(C)c2ccccc2-c2cc(N(c3ccc4cc5c(cc4c3)sc3ccccc35)c3ccc4sc5ccccc5c4c3)ccc21 ; CC1(C)c2ccccc2-c2cc(N(c3ccc4c(c3)-c3ccccc3C4(C)C)c3ccc4cc5c(cc4c3)sc3ccccc35)ccc21 ; CC1(C)c2ccccc2-c2cc(N(c3ccc(-c4ccc5cc6c(cc5c4)sc4ccccc46)cc3)c3ccc4c(c3)-c3ccccc3C4(C)C)ccc21] 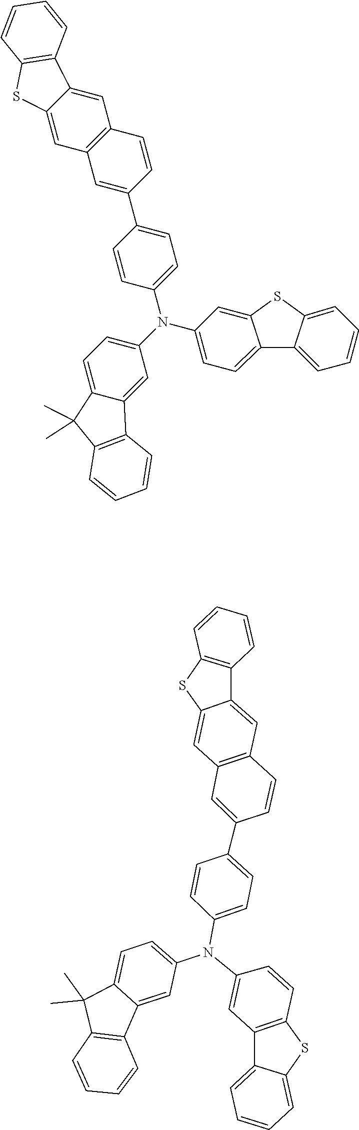 [SMILES: CC1(C)c2ccccc2-c2cc(N(c3ccc(-c4ccc5cc6c(cc5c4)sc4ccccc46)cc3)c3ccc4c(c3)sc3ccccc34)ccc21 ; CC1(C)c2ccccc2-c2cc(N(c3ccc(-c4ccc5cc6c(cc5c4)sc4ccccc46)cc3)c3ccc4sc5ccccc5c4c3)ccc21] 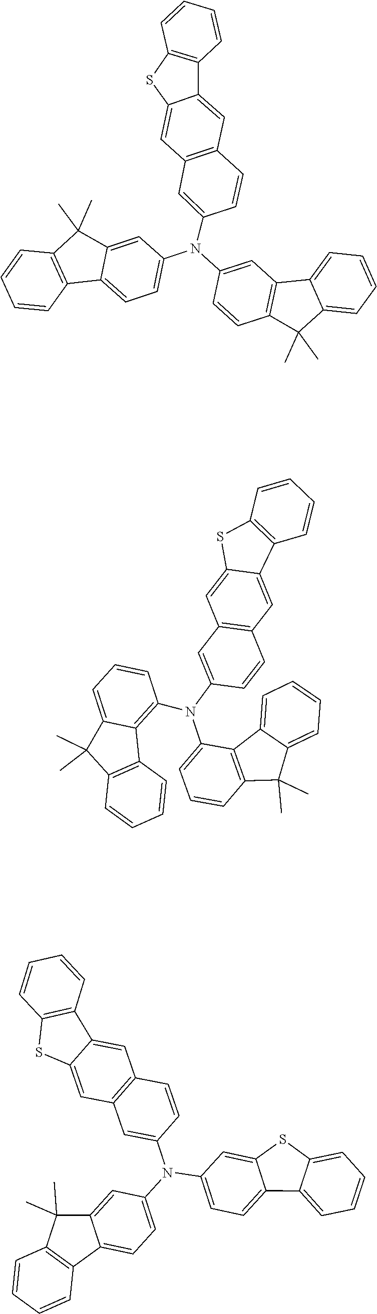 [SMILES: CC1(C)c2ccccc2-c2cc(N(c3ccc4c(c3)C(C)(C)c3ccccc3-4)c3ccc4cc5c(cc4c3)sc3ccccc35)ccc21 ; CC1(C)c2ccccc2-c2c(N(c3ccc4cc5c(cc4c3)sc3ccccc35)c3cccc4c3-c3ccccc3C4(C)C)cccc21 ; CC1(C)c2ccccc2-c2ccc(N(c3ccc4cc5c(cc4c3)sc3ccccc35)c3ccc4c(c3)sc3ccccc34)cc21] 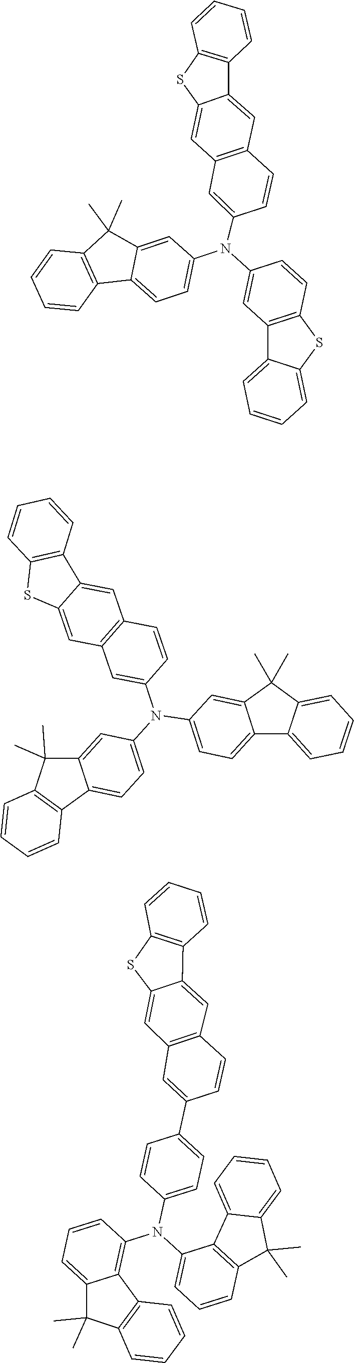 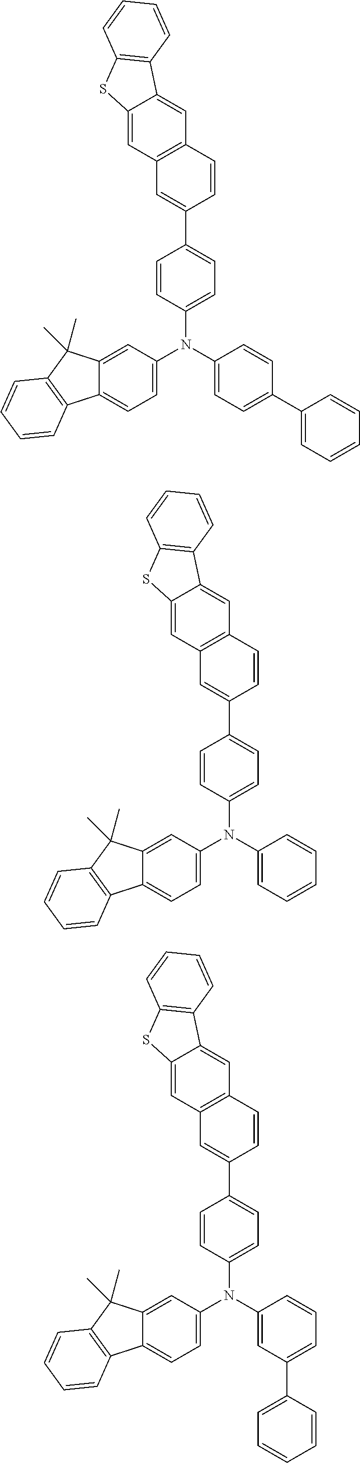 [SMILES: CC1(C)c2ccccc2-c2ccc(N(c3ccc(-c4ccccc4)cc3)c3ccc(-c4ccc5cc6c(cc5c4)sc4ccccc46)cc3)cc21 ; CC1(C)c2ccccc2-c2ccc(N(c3ccccc3)c3ccc(-c4ccc5cc6c(cc5c4)sc4ccccc46)cc3)cc21 ; CC1(C)c2ccccc2-c2ccc(N(c3ccc(-c4ccc5cc6c(cc5c4)sc4ccccc46)cc3)c3cccc(-c4ccccc4)c3)cc21] 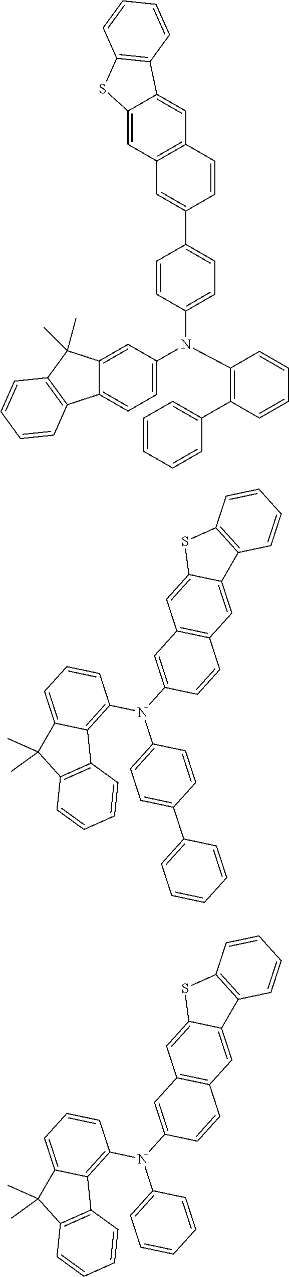 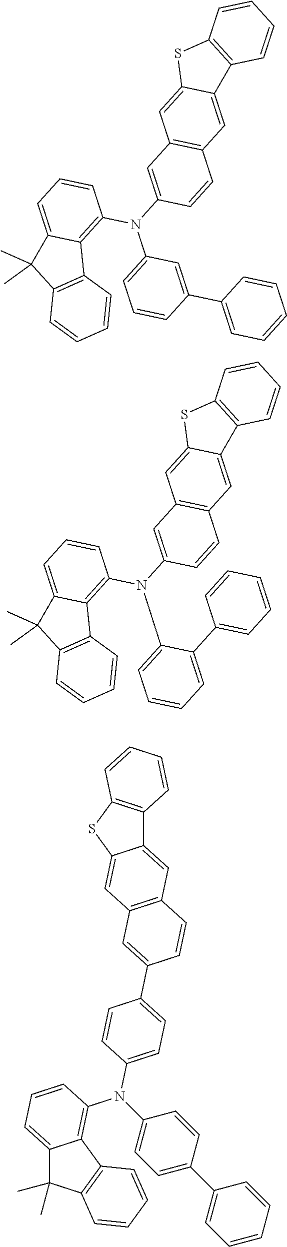 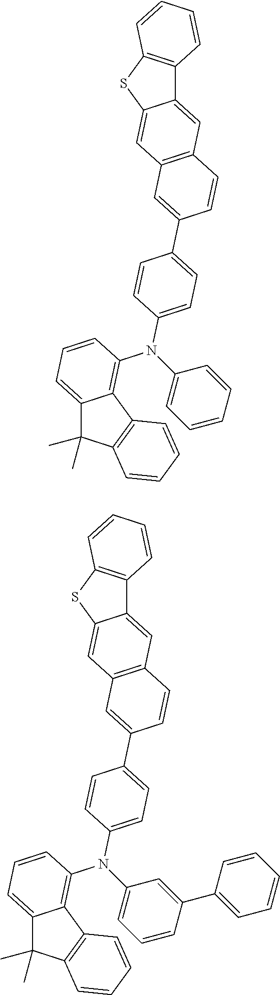 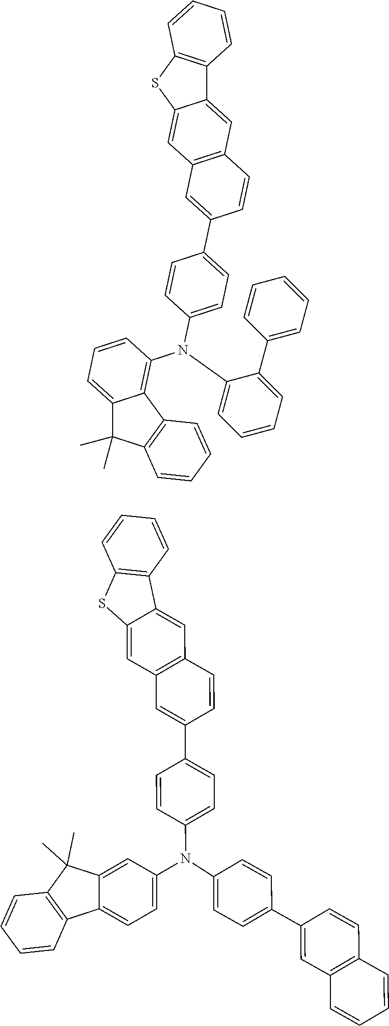 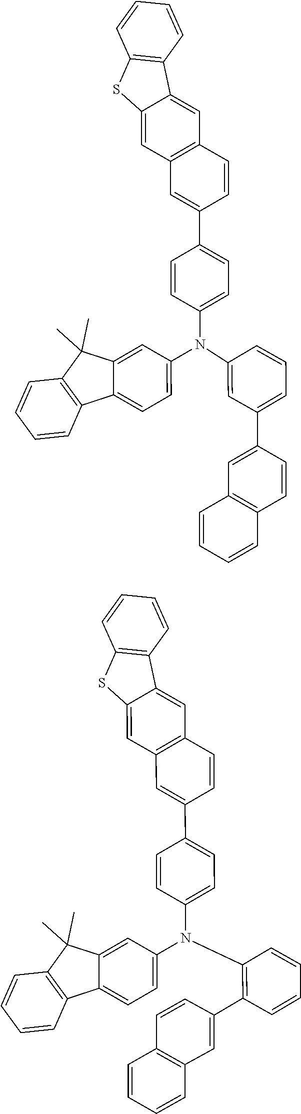 [SMILES: CC1(C)c2ccccc2-c2ccc(N(c3ccc(-c4ccc5cc6c(cc5c4)sc4ccccc46)cc3)c3cccc(-c4ccc5ccccc5c4)c3)cc21 ; CC1(C)c2ccccc2-c2ccc(N(c3ccc(-c4ccc5cc6c(cc5c4)sc4ccccc46)cc3)c3ccccc3-c3ccc4ccccc4c3)cc21] 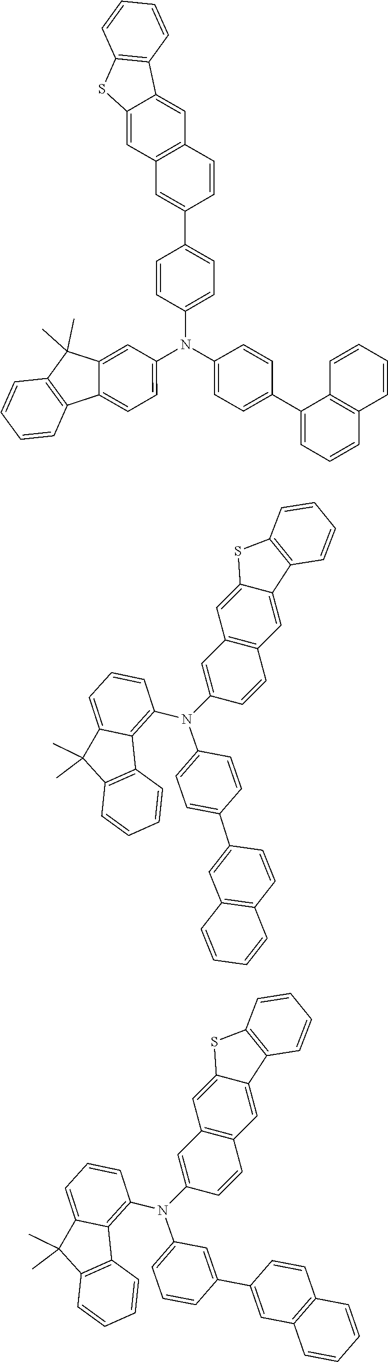 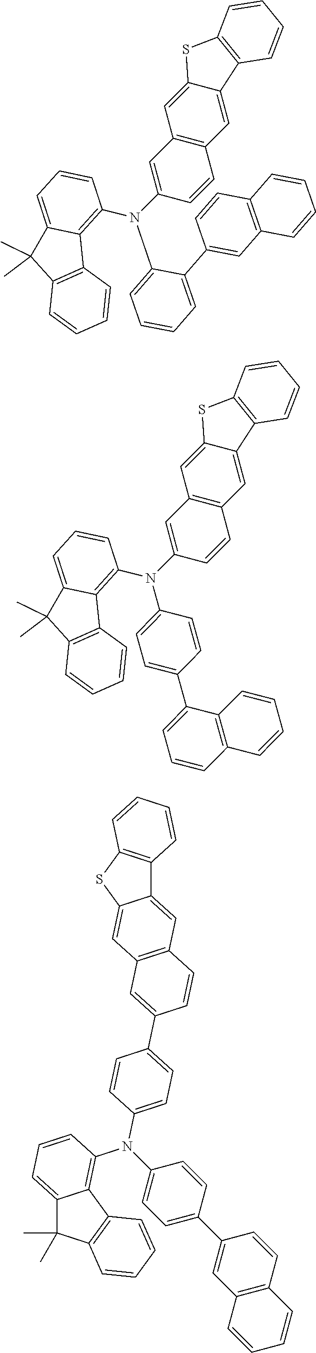 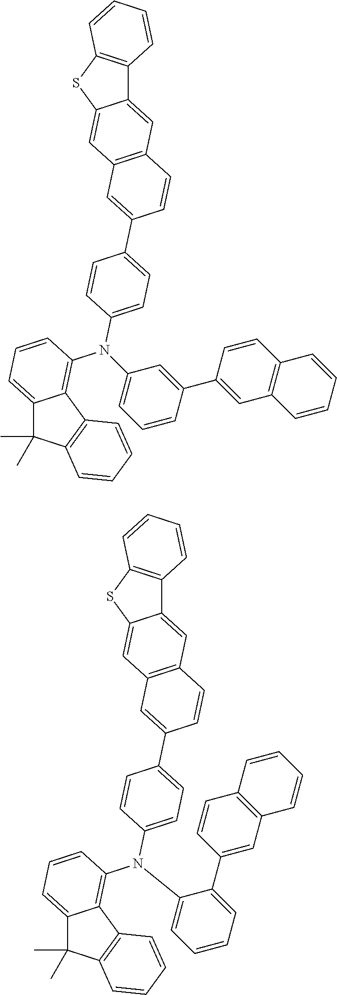 [SMILES: CC1(C)c2ccccc2-c2c(N(c3ccc(-c4ccc5cc6c(cc5c4)sc4ccccc46)cc3)c3cccc(-c4ccc5ccccc5c4)c3)cccc21 ; CC1(C)c2ccccc2-c2c(N(c3ccc(-c4ccc5cc6c(cc5c4)sc4ccccc46)cc3)c3ccccc3-c3ccc4ccccc4c3)cccc21] 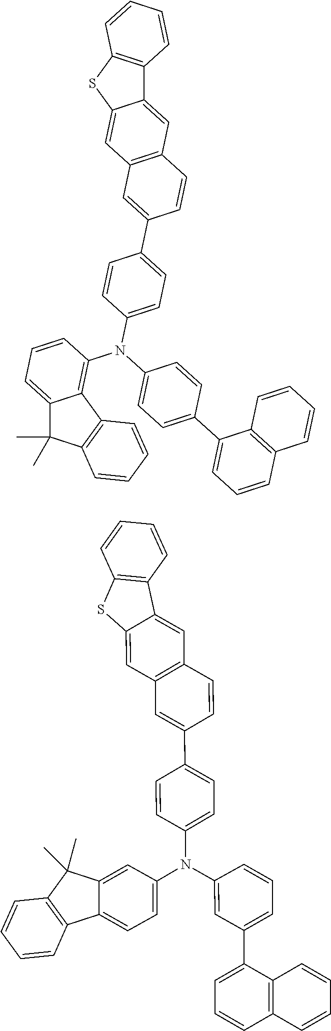 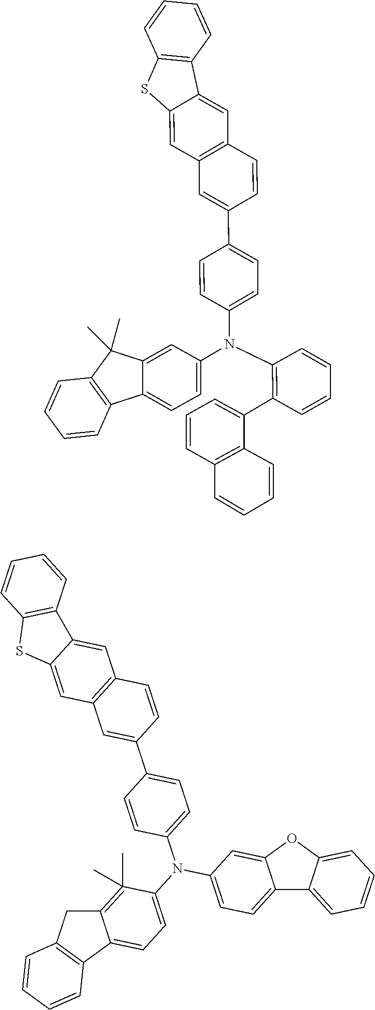 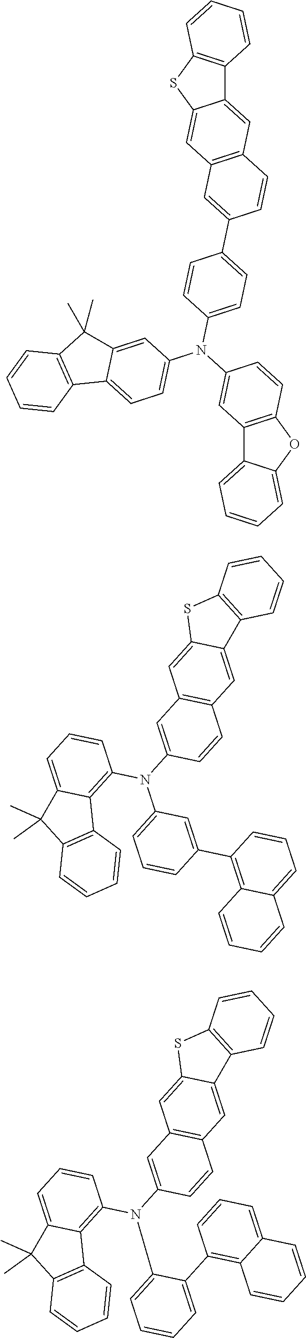 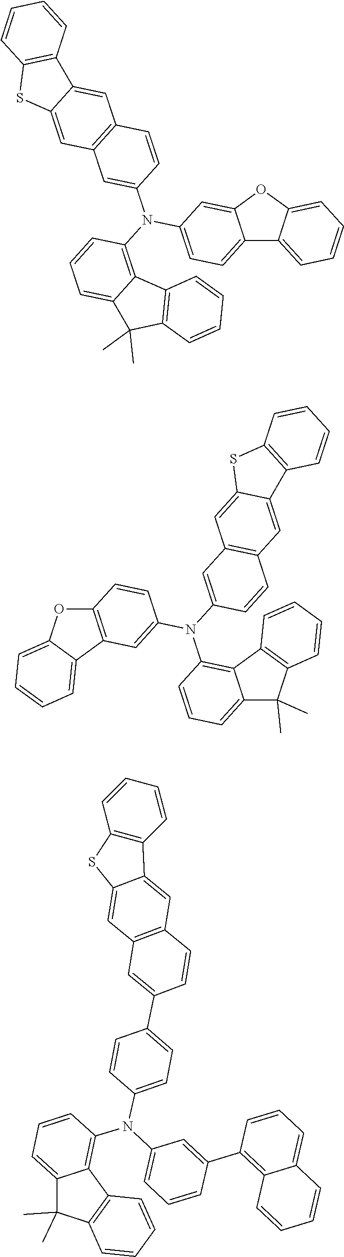 [SMILES: CC1(C)c2ccccc2-c2c(N(c3ccc4cc5c(cc4c3)sc3ccccc35)c3ccc4c(c3)oc3ccccc34)cccc21 ; CC1(C)c2ccccc2-c2c(N(c3ccc4cc5c(cc4c3)sc3ccccc35)c3ccc4oc5ccccc5c4c3)cccc21 ; CC1(C)c2ccccc2-c2c(N(c3ccc(-c4ccc5cc6c(cc5c4)sc4ccccc46)cc3)c3cccc(-c4cccc5ccccc45)c3)cccc21] 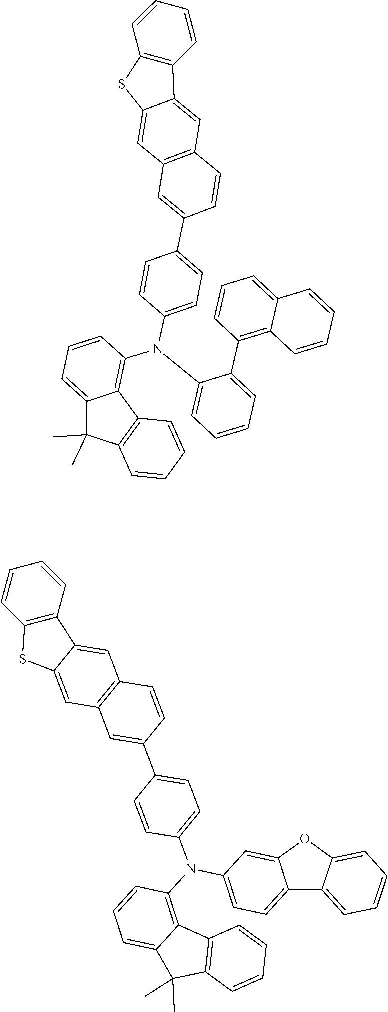 [SMILES: CC1(C)c2ccccc2-c2c(N(c3ccc(-c4ccc5cc6c(cc5c4)sc4ccccc46)cc3)c3ccccc3-c3cccc4ccccc34)cccc21 ; CC1(C)c2ccccc2-c2c(N(c3ccc(-c4ccc5cc6c(cc5c4)sc4ccccc46)cc3)c3ccc4c(c3)oc3ccccc34)cccc21] 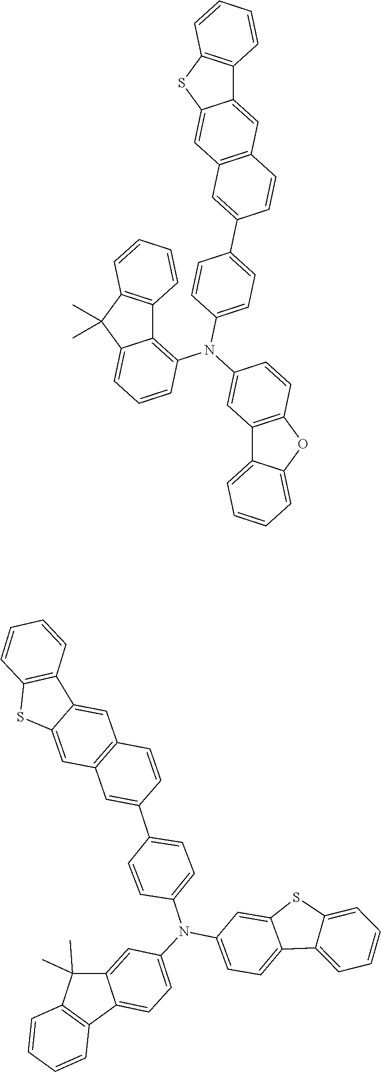 [SMILES: CC1(C)c2ccccc2-c2c(N(c3ccc(-c4ccc5cc6c(cc5c4)sc4ccccc46)cc3)c3ccc4oc5ccccc5c4c3)cccc21 ; CC1(C)c2ccccc2-c2ccc(N(c3ccc(-c4ccc5cc6c(cc5c4)sc4ccccc46)cc3)c3ccc4c(c3)sc3ccccc34)cc21] 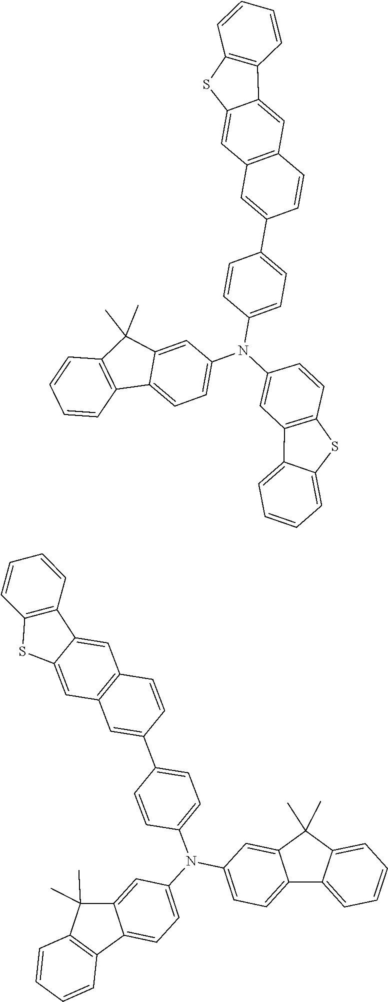 [SMILES: CC1(C)c2ccccc2-c2ccc(N(c3ccc(-c4ccc5cc6c(cc5c4)sc4ccccc46)cc3)c3ccc4sc5ccccc5c4c3)cc21 ; CC1(C)c2ccccc2-c2ccc(N(c3ccc(-c4ccc5cc6c(cc5c4)sc4ccccc46)cc3)c3ccc4c(c3)C(C)(C)c3ccccc3-4)cc21] 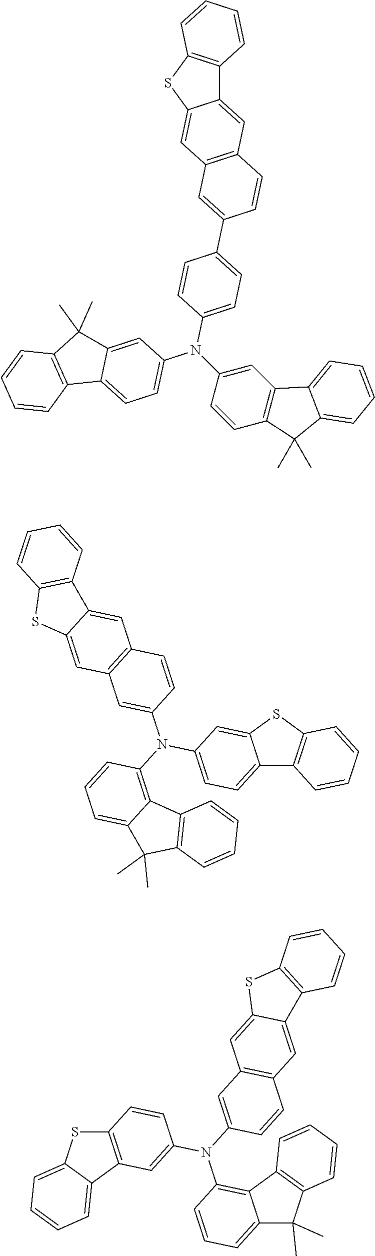 [SMILES: CC1(C)c2ccccc2-c2cc(N(c3ccc(-c4ccc5cc6c(cc5c4)sc4ccccc46)cc3)c3ccc4c(c3)C(C)(C)c3ccccc3-4)ccc21 ; CC1(C)c2ccccc2-c2c(N(c3ccc4cc5c(cc4c3)sc3ccccc35)c3ccc4c(c3)sc3ccccc34)cccc21 ; CC1(C)c2ccccc2-c2c(N(c3ccc4cc5c(cc4c3)sc3ccccc35)c3ccc4sc5ccccc5c4c3)cccc21] 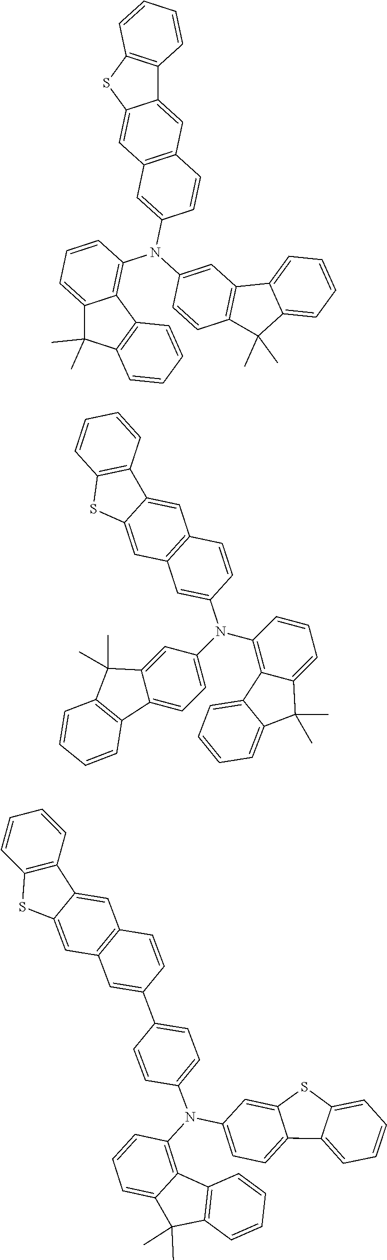 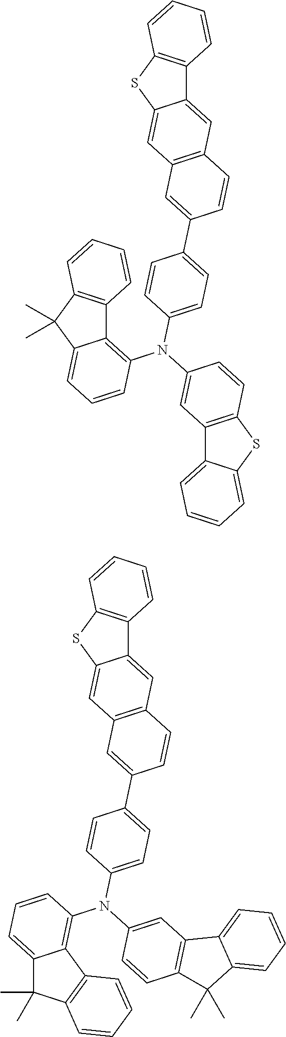 [SMILES: CC1(C)c2ccccc2-c2c(N(c3ccc(-c4ccc5cc6c(cc5c4)sc4ccccc46)cc3)c3ccc4sc5ccccc5c4c3)cccc21 ; CC1(C)c2ccccc2-c2cc(N(c3ccc(-c4ccc5cc6c(cc5c4)sc4ccccc46)cc3)c3cccc4c3-c3ccccc3C4(C)C)ccc21] 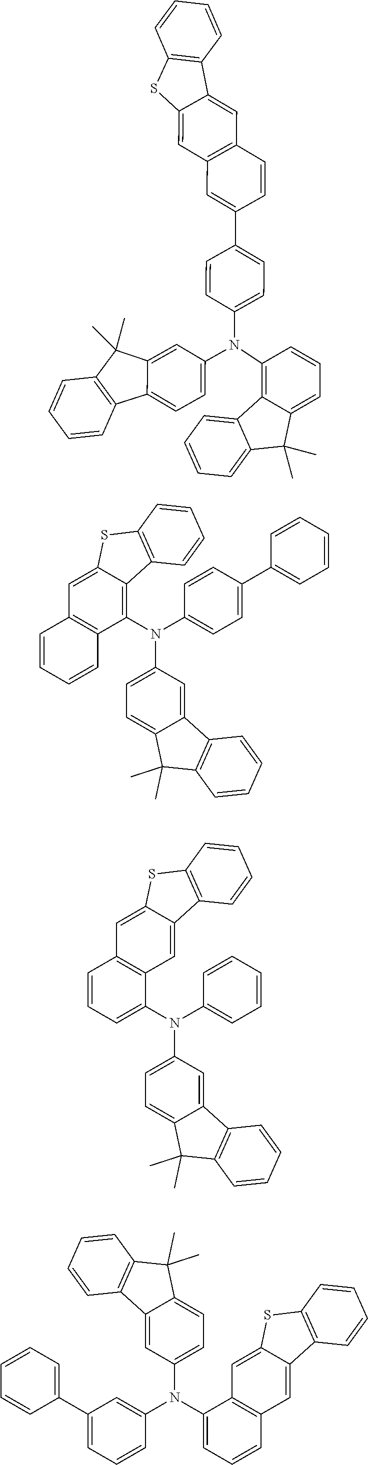 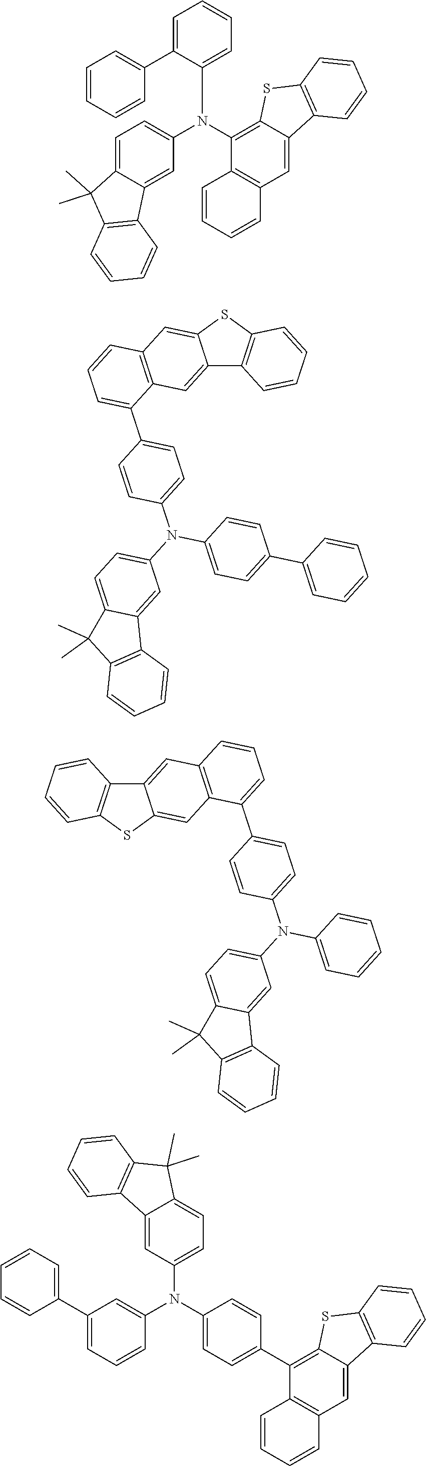 [SMILES: CC1(C)c2ccccc2-c2cc(N(c3ccccc3-c3ccccc3)c3c4ccccc4cc4c3sc3ccccc34)ccc21 ; CC1(C)c2ccccc2-c2cc(N(c3ccc(-c4ccccc4)cc3)c3ccc(-c4cccc5cc6sc7ccccc7c6cc45)cc3)ccc21 ; CC1(C)c2ccccc2-c2cc(N(c3ccccc3)c3ccc(-c4cccc5cc6c(cc45)sc4ccccc46)cc3)ccc21 ; CC1(C)c2ccccc2-c2cc(N(c3ccc(-c4c5ccccc5cc5c4sc4ccccc45)cc3)c3cccc(-c4ccccc4)c3)ccc21] 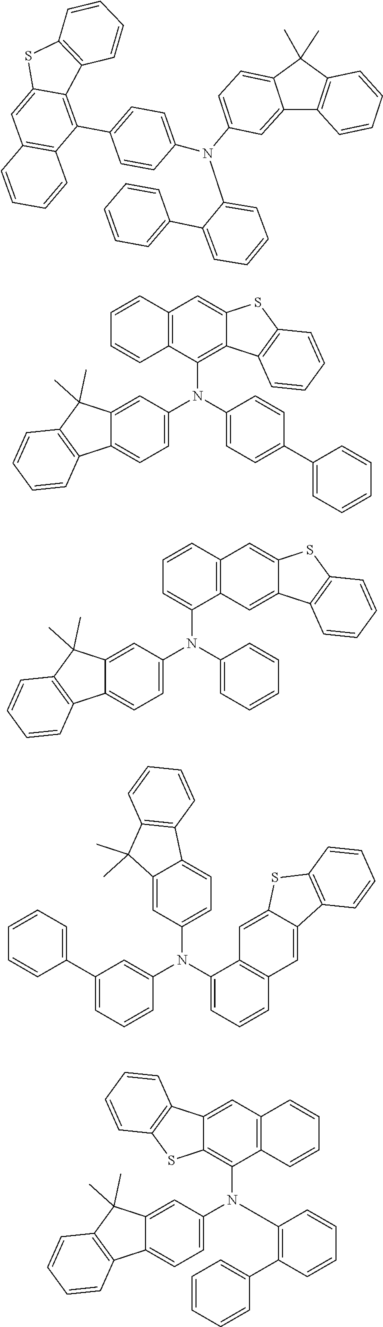 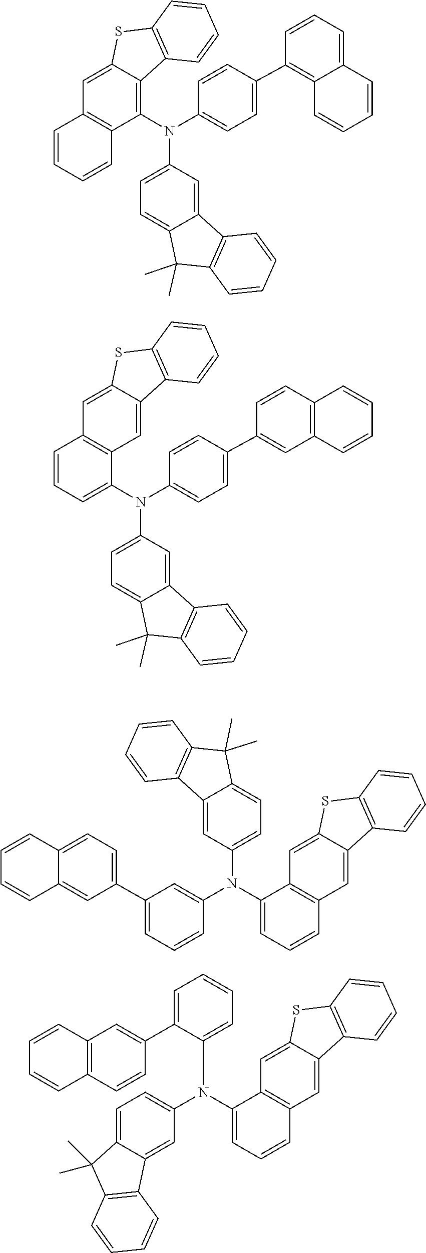 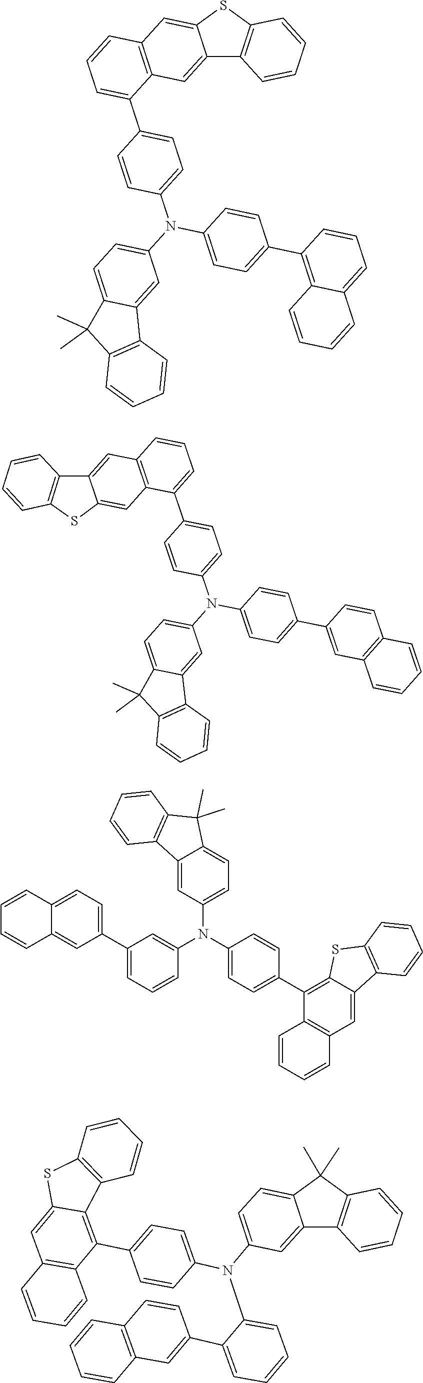 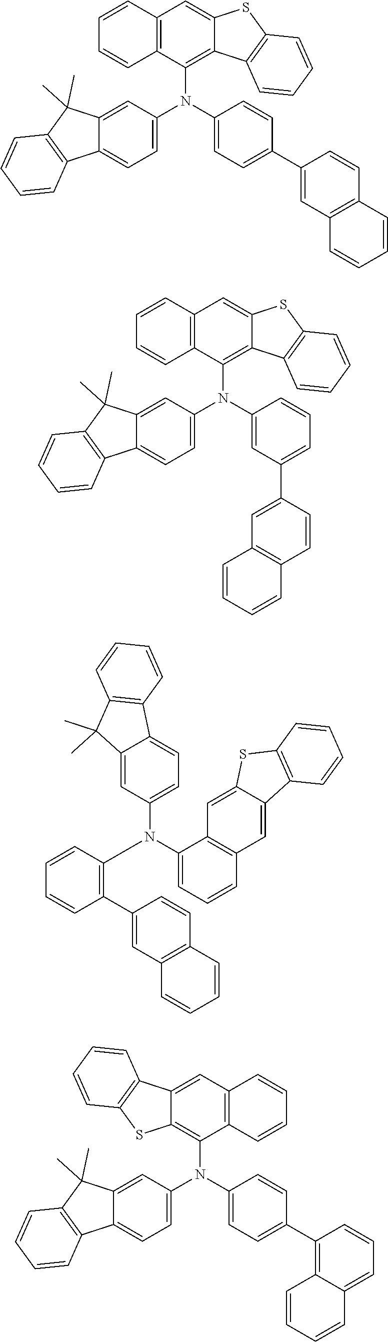 [SMILES: CC1(C)c2ccccc2-c2ccc(N(c3ccc(-c4ccc5ccccc5c4)cc3)c3c4ccccc4cc4sc5ccccc5c34)cc21 ; CC1(C)c2ccccc2-c2ccc(N(c3cccc(-c4ccc5ccccc5c4)c3)c3c4ccccc4cc4sc5ccccc5c34)cc21 ; CC1(C)c2ccccc2-c2ccc(N(c3ccccc3-c3ccc4ccccc4c3)c3cccc4cc5c(cc34)sc3ccccc35)cc21 ; CC1(C)c2ccccc2-c2ccc(N(c3ccc(-c4cccc5ccccc45)cc3)c3c4ccccc4cc4c3sc3ccccc34)cc21] 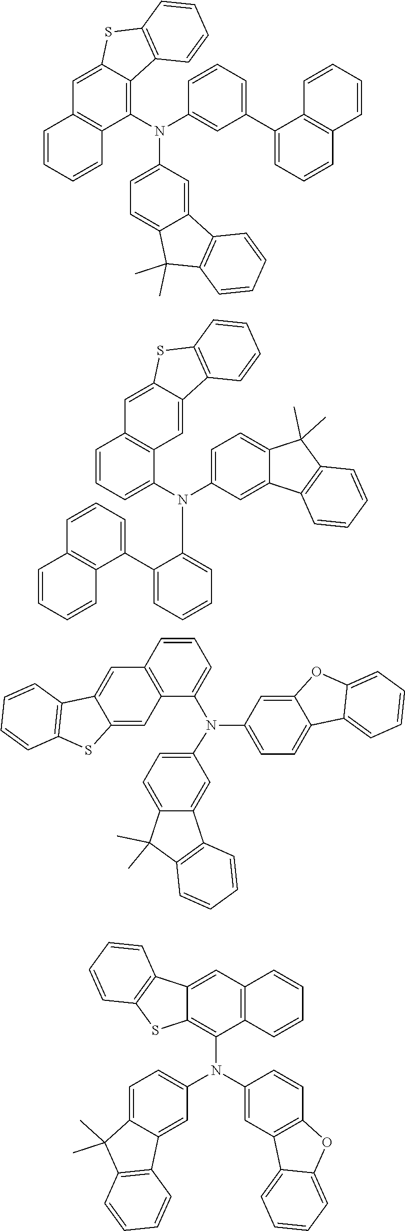 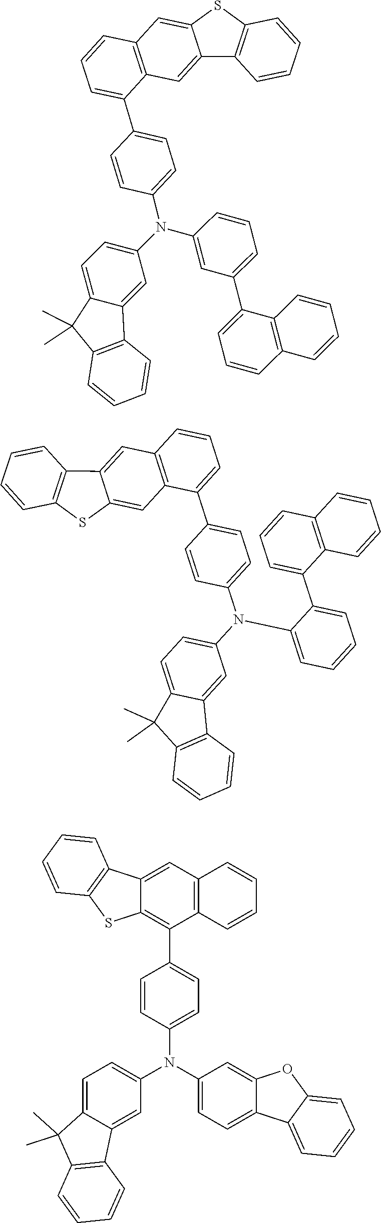 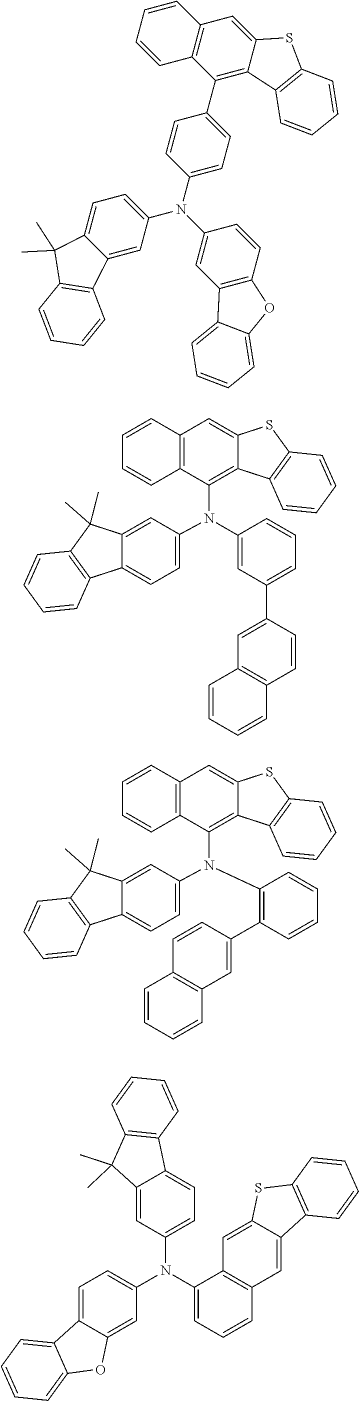 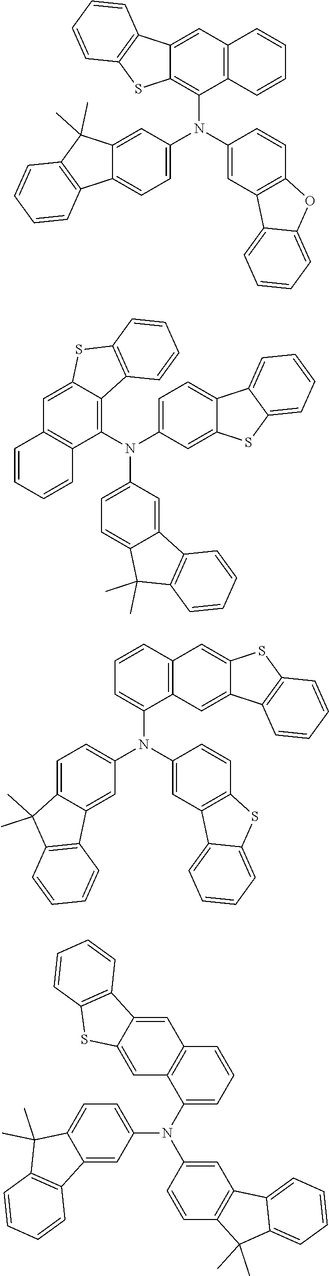 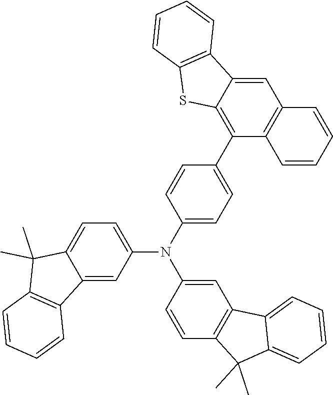 [SMILES: CC1(C)c2ccccc2-c2cc(N(c3ccc(-c4c5ccccc5cc5c4sc4ccccc45)cc3)c3ccc4c(c3)-c3ccccc3C4(C)C)ccc21] 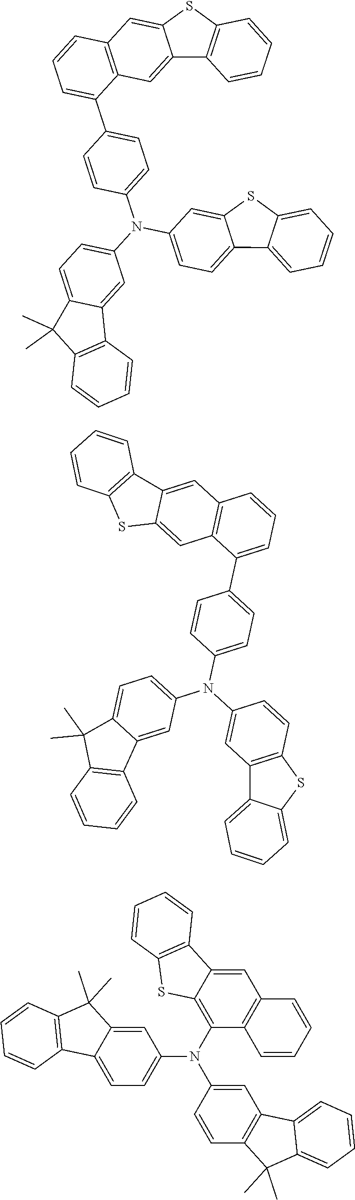 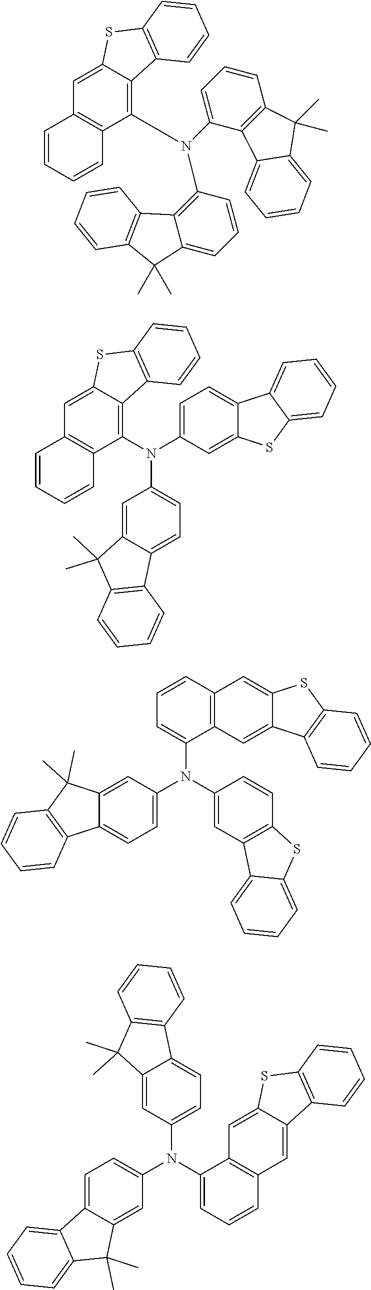 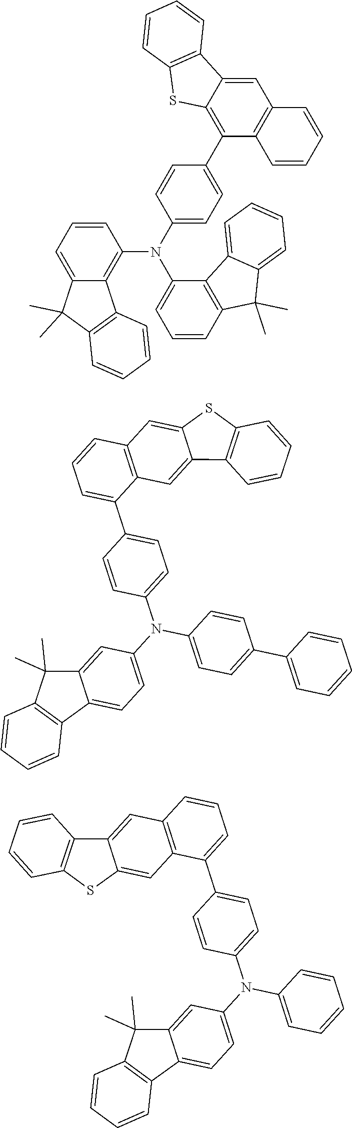 [SMILES: CC1(C)c2ccccc2-c2c(N(c3ccc(-c4c5ccccc5cc5c4sc4ccccc45)cc3)c3cccc4c3-c3ccccc3C4(C)C)cccc21 ; CC1(C)c2ccccc2-c2ccc(N(c3ccc(-c4ccccc4)cc3)c3ccc(-c4cccc5cc6sc7ccccc7c6cc45)cc3)cc21 ; CC1(C)c2ccccc2-c2ccc(N(c3ccccc3)c3ccc(-c4cccc5cc6c(cc45)sc4ccccc46)cc3)cc21] 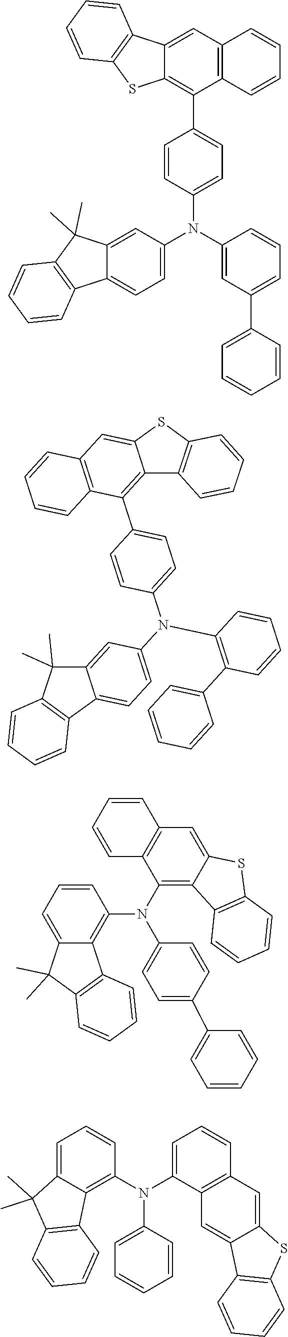 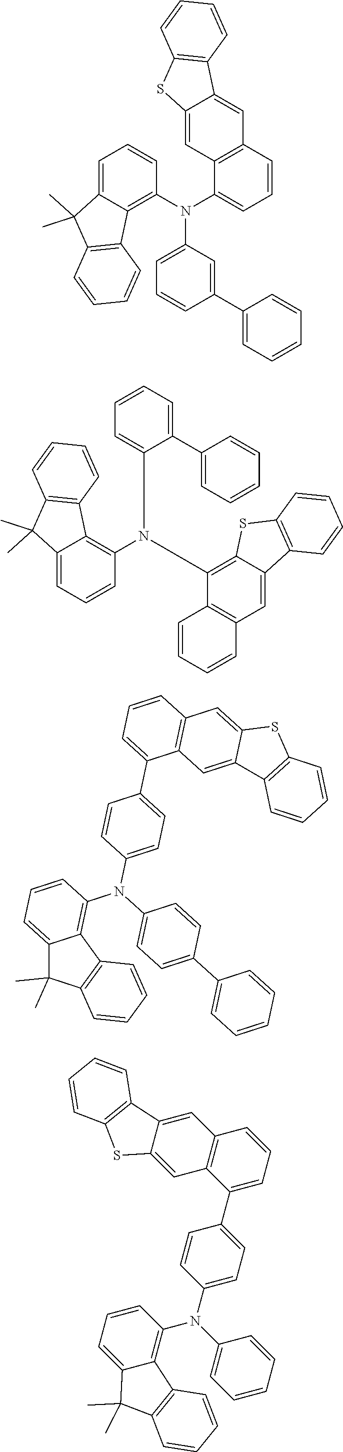 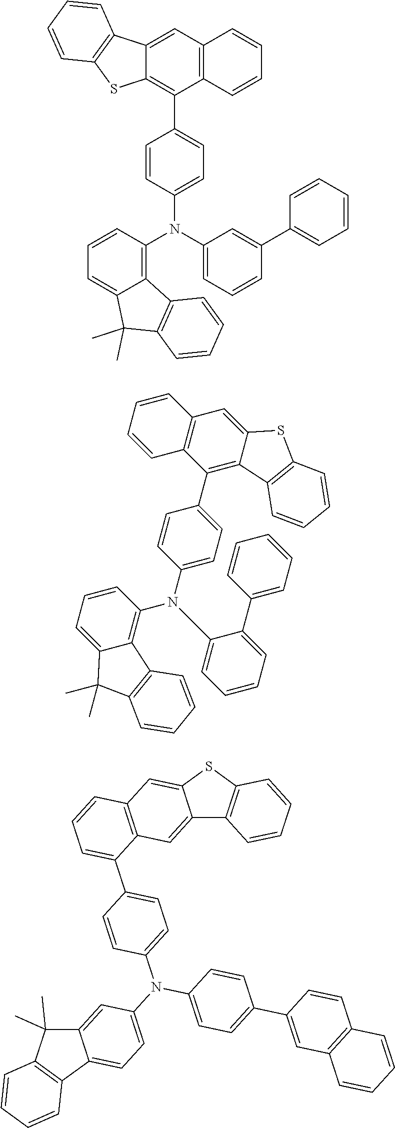 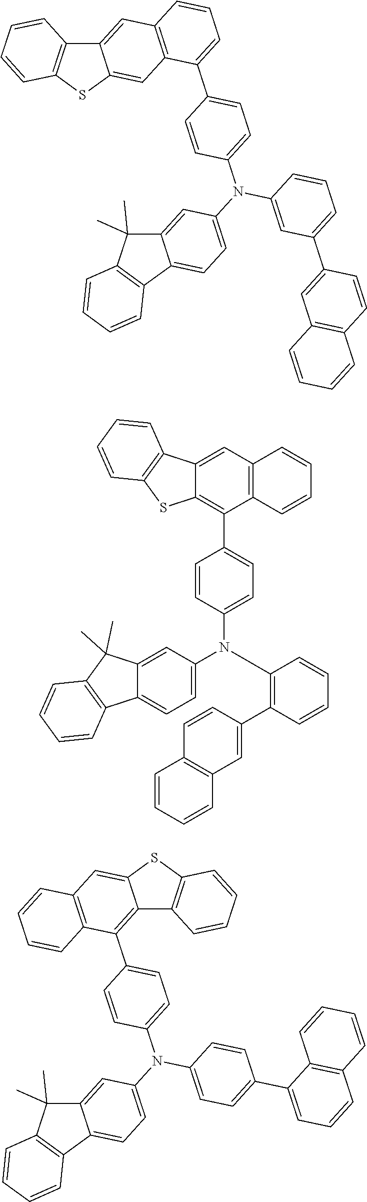 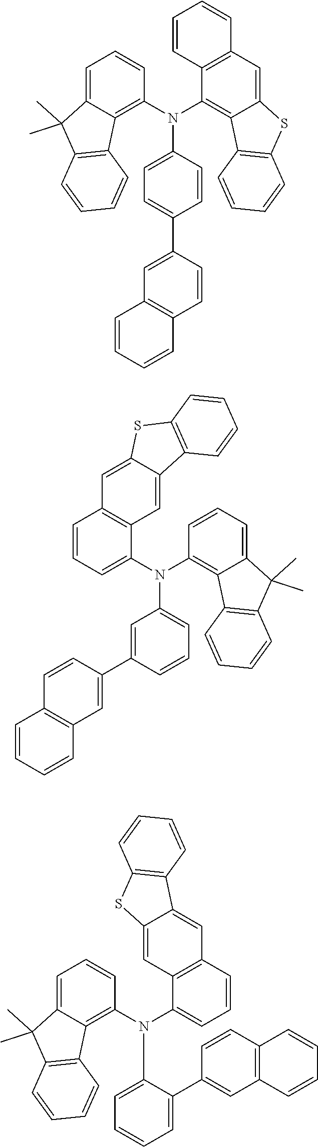 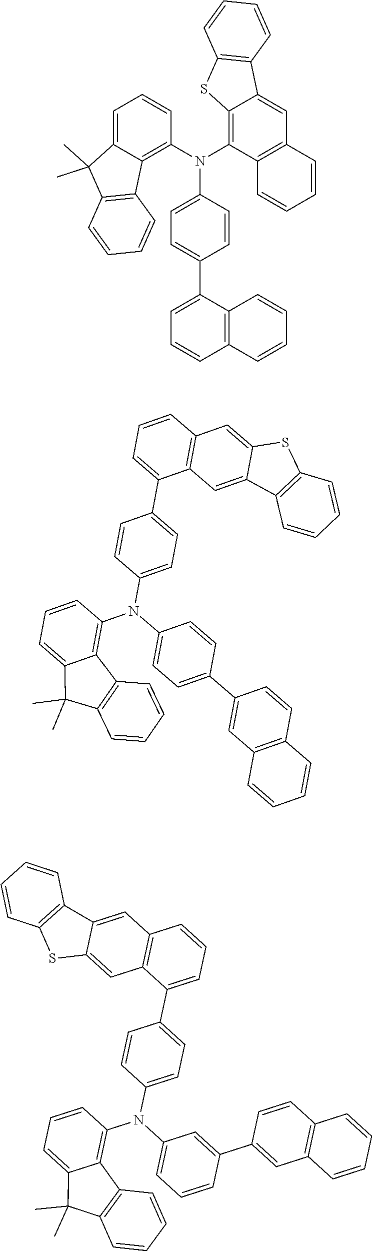 [SMILES: CC1(C)c2ccccc2-c2c(N(c3ccc(-c4cccc5ccccc45)cc3)c3c4ccccc4cc4c3sc3ccccc34)cccc21 ; CC1(C)c2ccccc2-c2c(N(c3ccc(-c4ccc5ccccc5c4)cc3)c3ccc(-c4cccc5cc6sc7ccccc7c6cc45)cc3)cccc21 ; CC1(C)c2ccccc2-c2c(N(c3ccc(-c4cccc5cc6c(cc45)sc4ccccc46)cc3)c3cccc(-c4ccc5ccccc5c4)c3)cccc21] 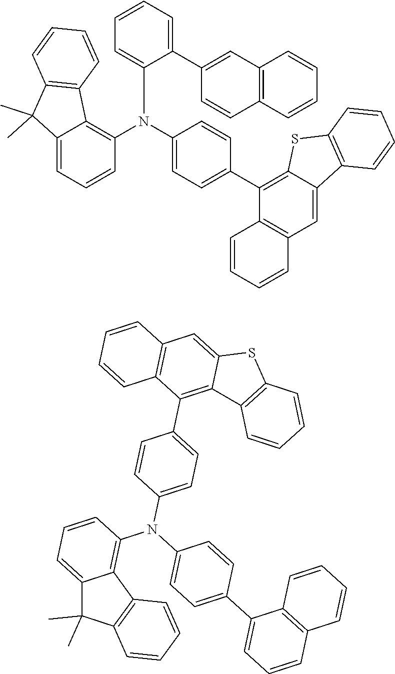 [SMILES: CC1(C)c2ccccc2-c2c(N(c3ccc(-c4c5ccccc5cc5c4sc4ccccc45)cc3)c3ccccc3-c3ccc4ccccc4c3)cccc21 ; CC1(C)c2ccccc2-c2c(N(c3ccc(-c4cccc5ccccc45)cc3)c3ccc(-c4c5ccccc5cc5sc6ccccc6c45)cc3)cccc21] 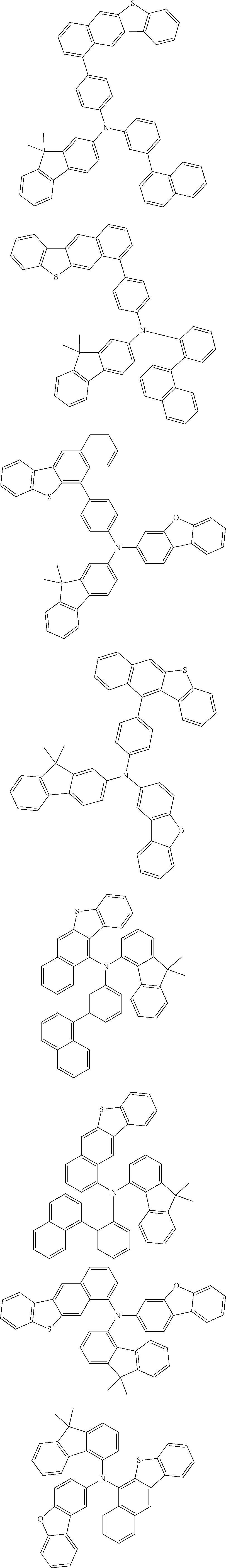 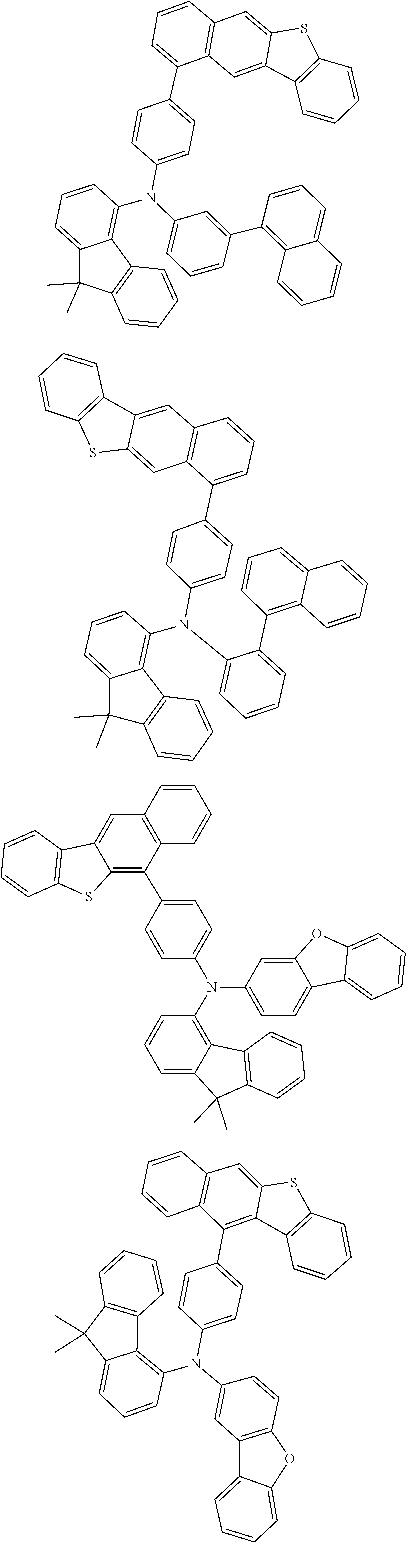 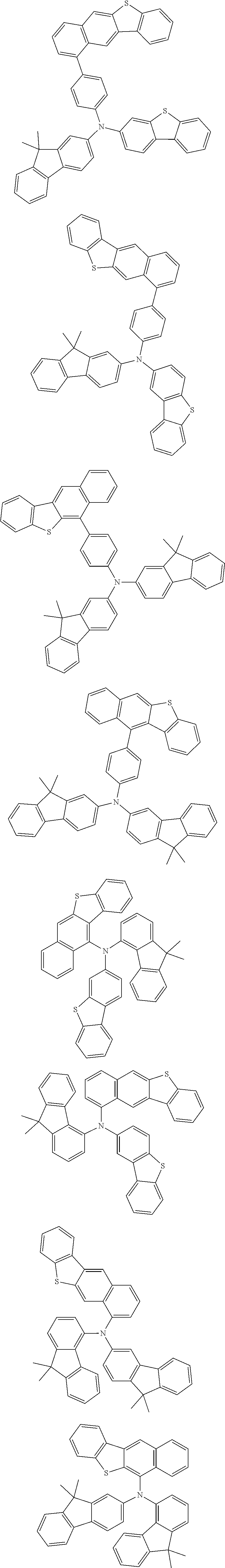 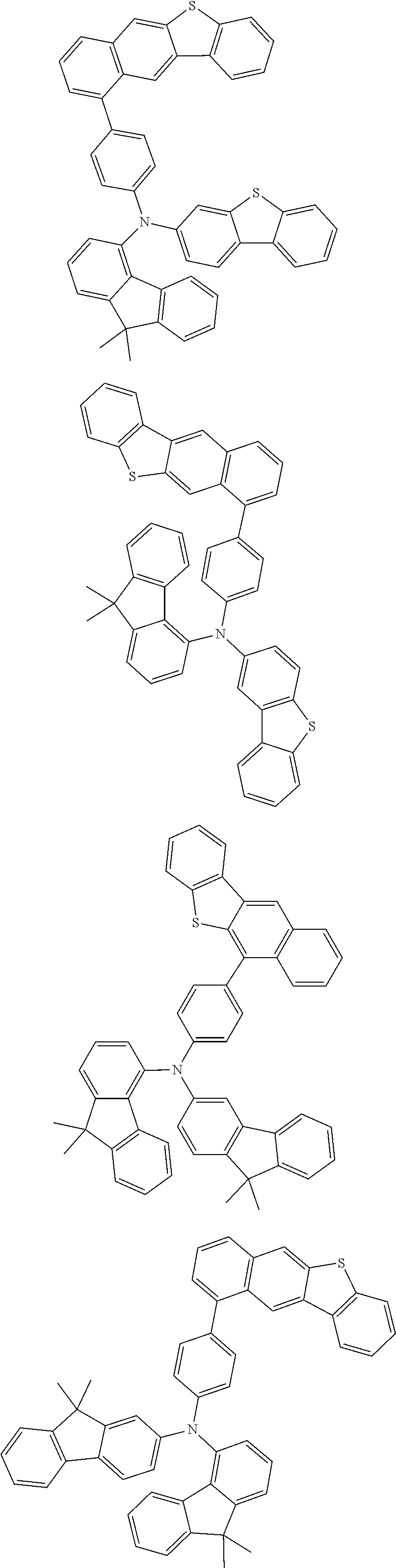 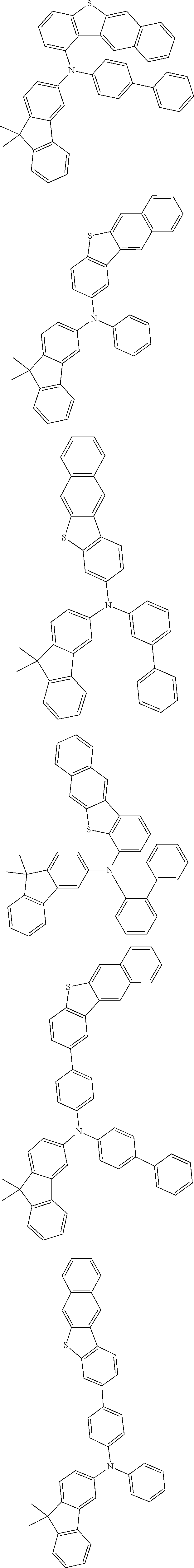 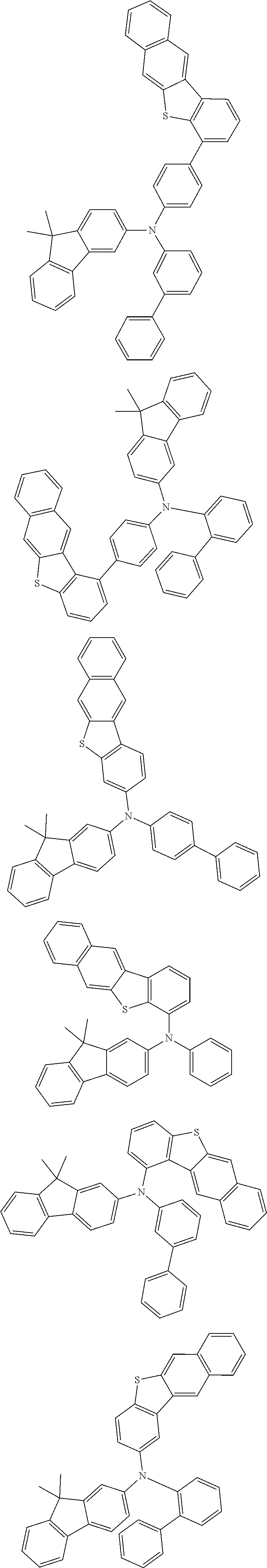 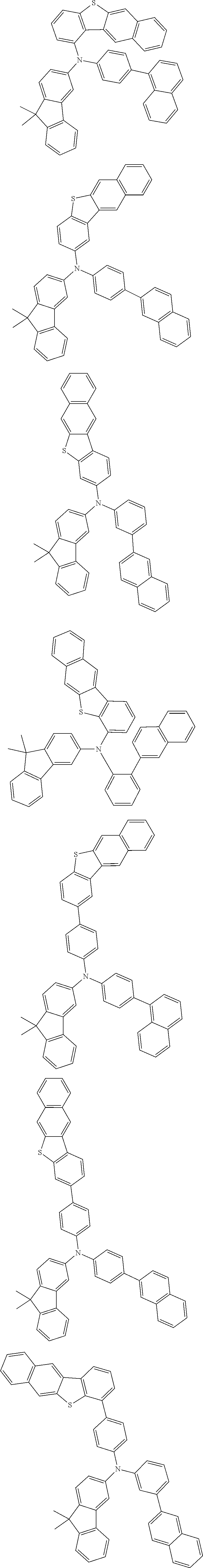 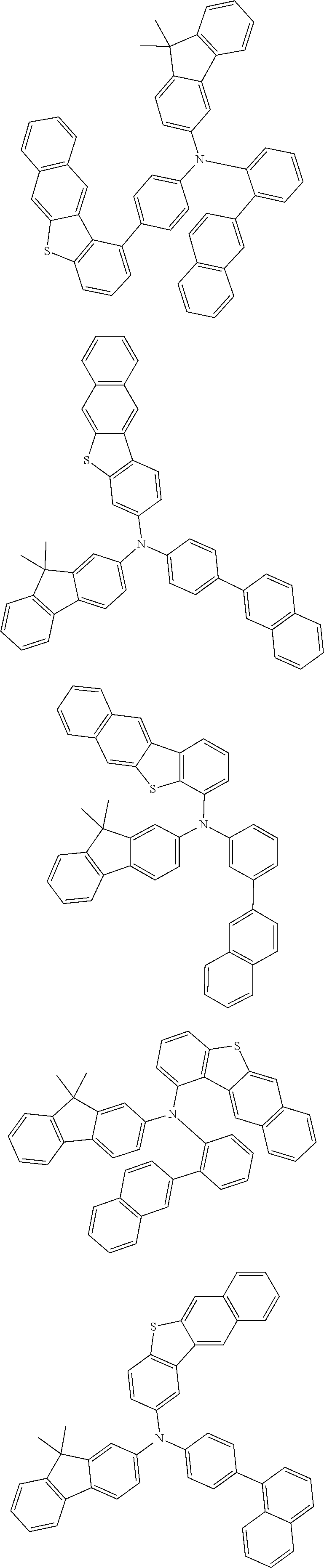 [SMILES: CC1(C)c2ccccc2-c2cc(N(c3ccc(-c4cccc5sc6cc7ccccc7cc6c45)cc3)c3ccccc3-c3ccc4ccccc4c3)ccc21 ; CC1(C)c2ccccc2-c2ccc(N(c3ccc(-c4ccc5ccccc5c4)cc3)c3ccc4c(c3)sc3cc5ccccc5cc34)cc21 ; CC1(C)c2ccccc2-c2ccc(N(c3cccc(-c4ccc5ccccc5c4)c3)c3cccc4c3sc3cc5ccccc5cc34)cc21 ; CC1(C)c2ccccc2-c2ccc(N(c3ccccc3-c3ccc4ccccc4c3)c3cccc4sc5cc6ccccc6cc5c34)cc21 ; CC1(C)c2ccccc2-c2ccc(N(c3ccc(-c4cccc5ccccc45)cc3)c3ccc4sc5cc6ccccc6cc5c4c3)cc21] 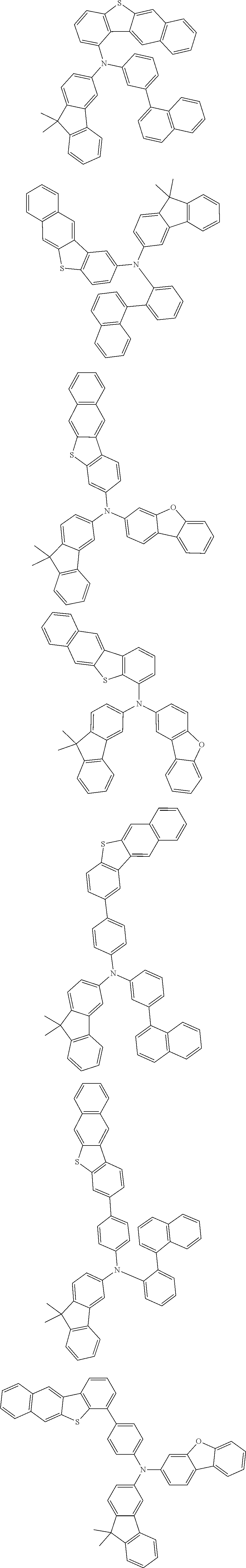 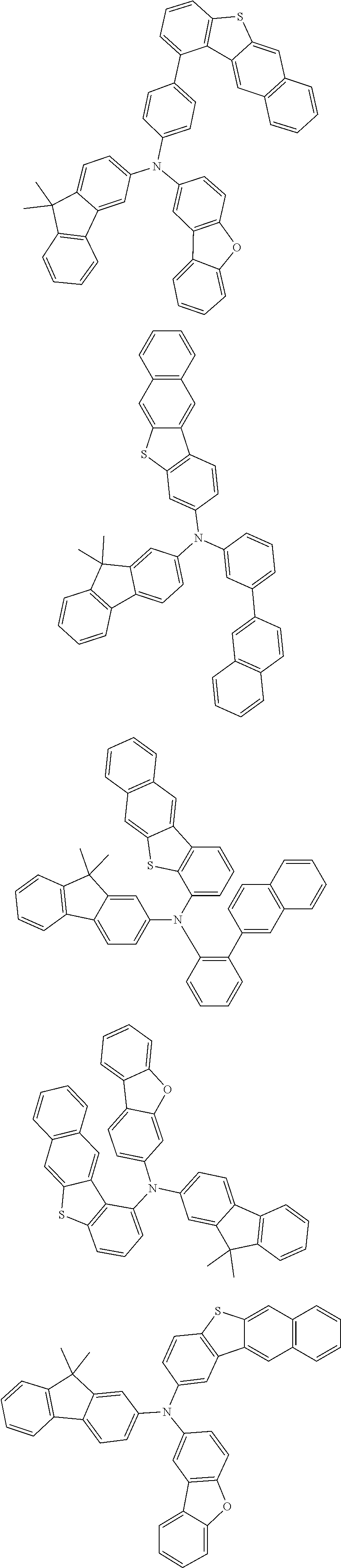 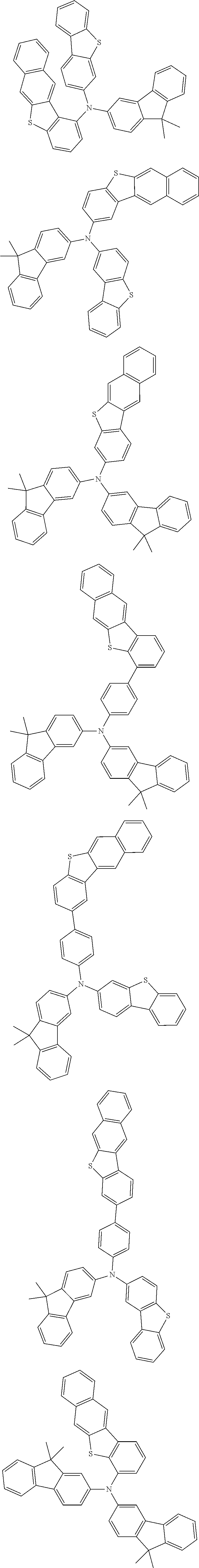 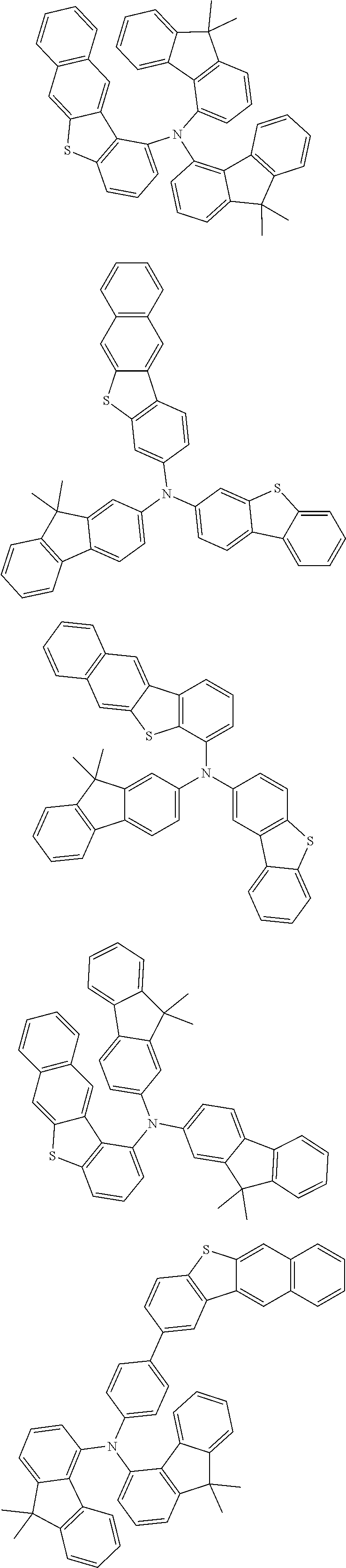 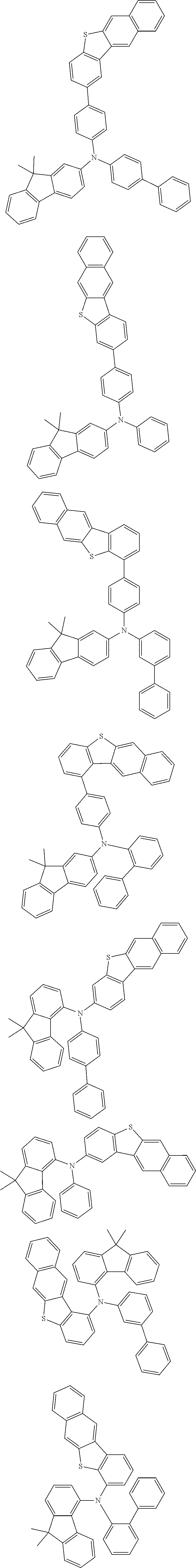 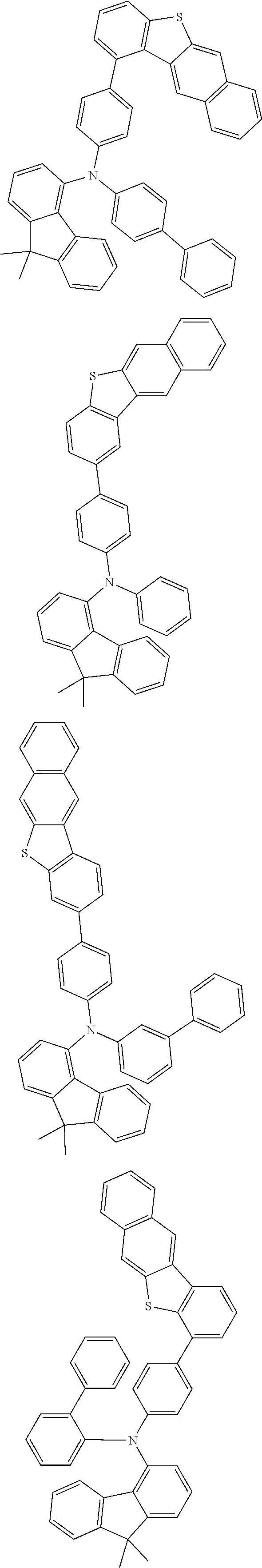 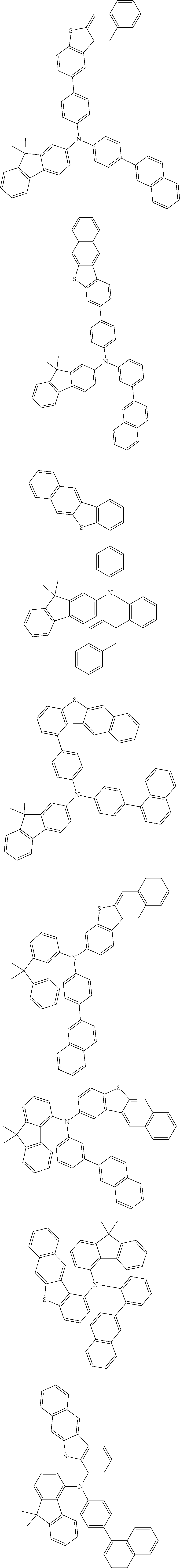 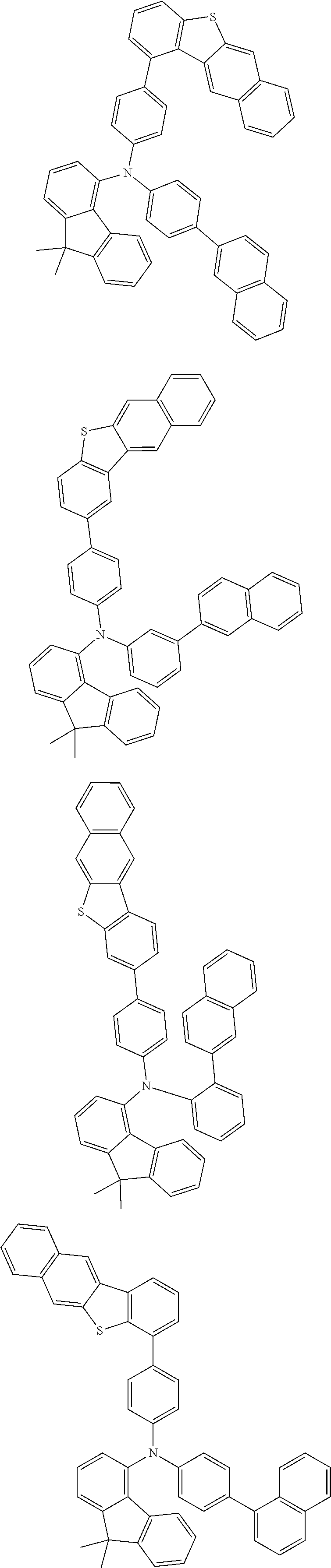 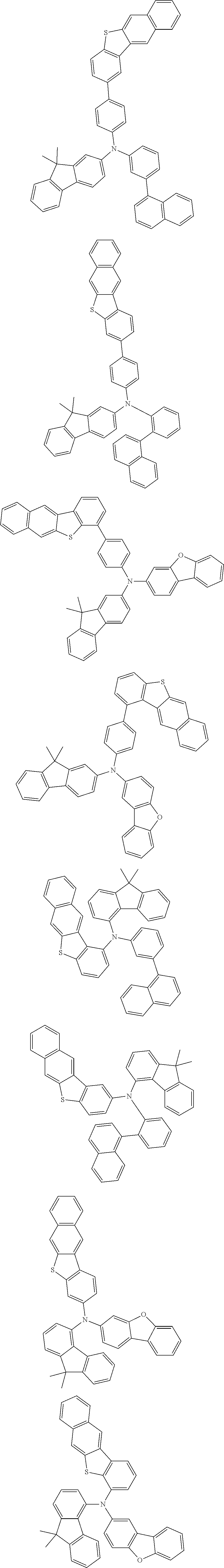 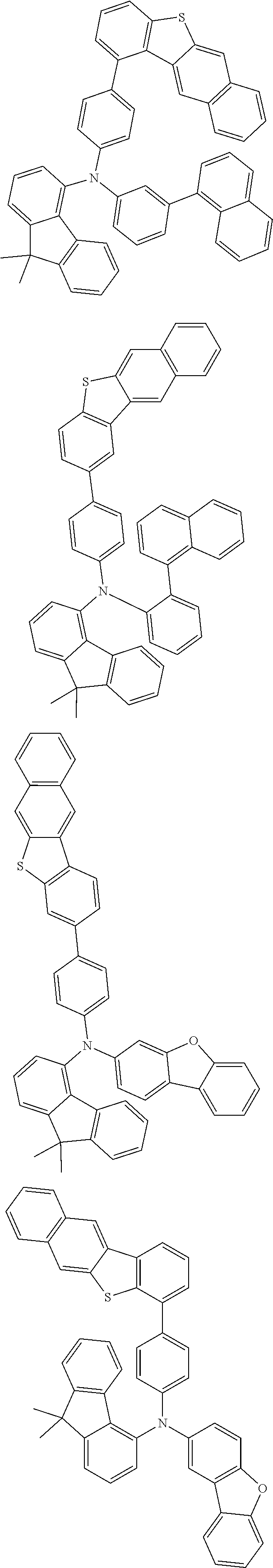 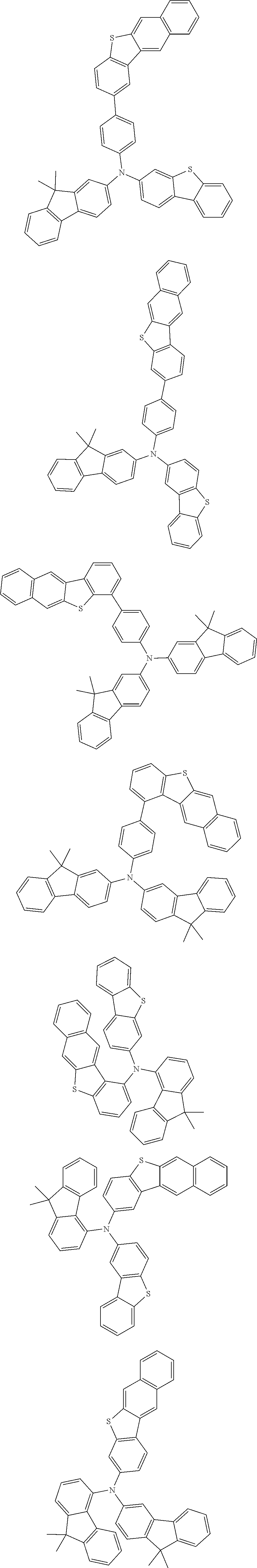 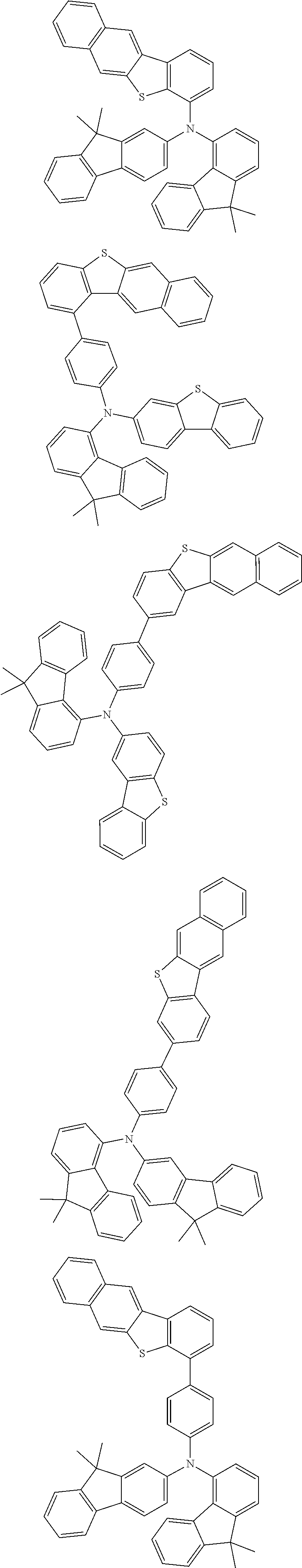 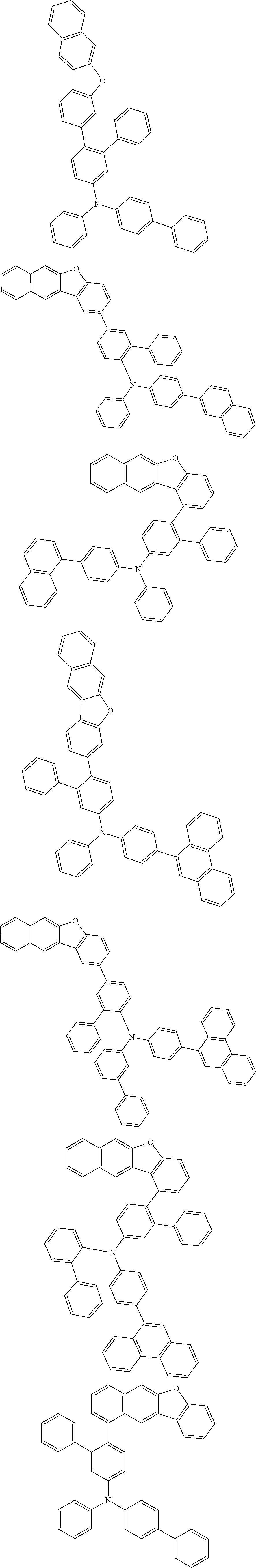 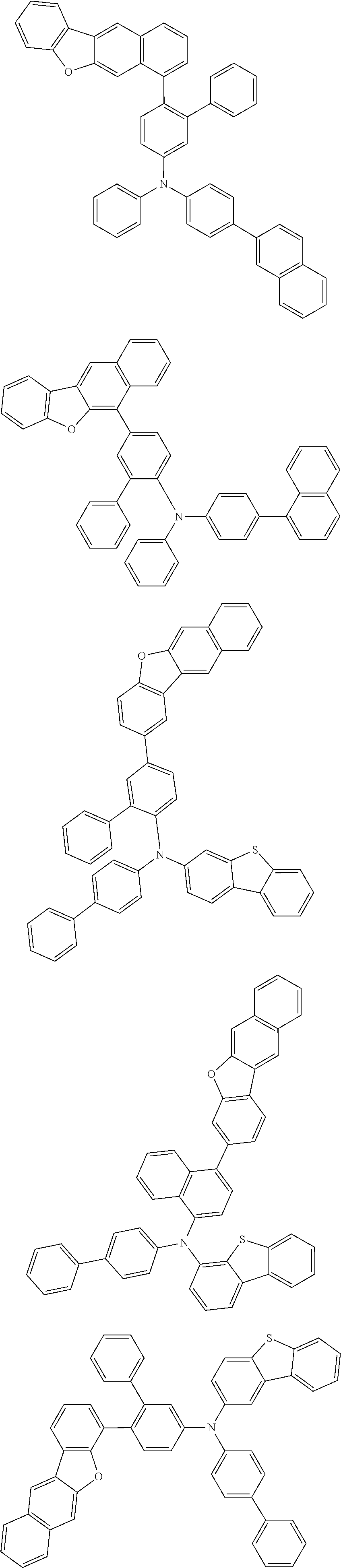 [SMILES: c1ccc(-c2cc(N(c3ccccc3)c3ccc(-c4ccc5ccccc5c4)cc3)ccc2-c2cccc3cc4c(cc23)oc2ccccc24)cc1 ; c1ccc(-c2cc(-c3c4ccccc4cc4c3oc3ccccc34)ccc2N(c2ccccc2)c2ccc(-c3cccc4ccccc34)cc2)cc1 ; c1ccc(-c2ccc(N(c3ccc4c(c3)sc3ccccc34)c3ccc(-c4ccc5oc6cc7ccccc7cc6c5c4)cc3-c3ccccc3)cc2)cc1 ; c1ccc(-c2ccc(N(c3ccc(-c4ccc5c(c4)oc4cc6ccccc6cc45)c4ccccc34)c3cccc4c3sc3ccccc34)cc2)cc1 ; c1ccc(-c2ccc(N(c3ccc(-c4cccc5c4oc4cc6ccccc6cc45)c(-c4ccccc4)c3)c3ccc4sc5ccccc5c4c3)cc2)cc1] 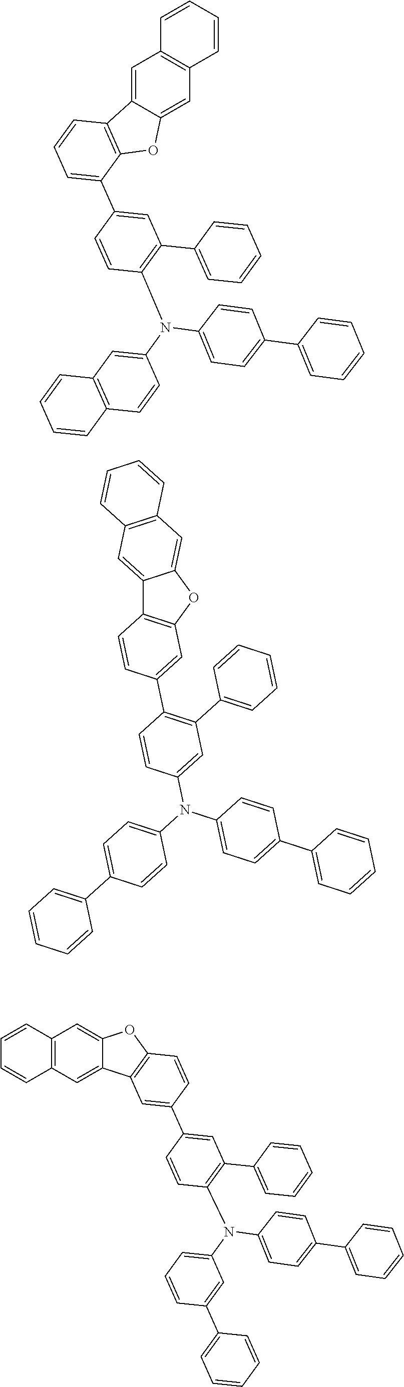 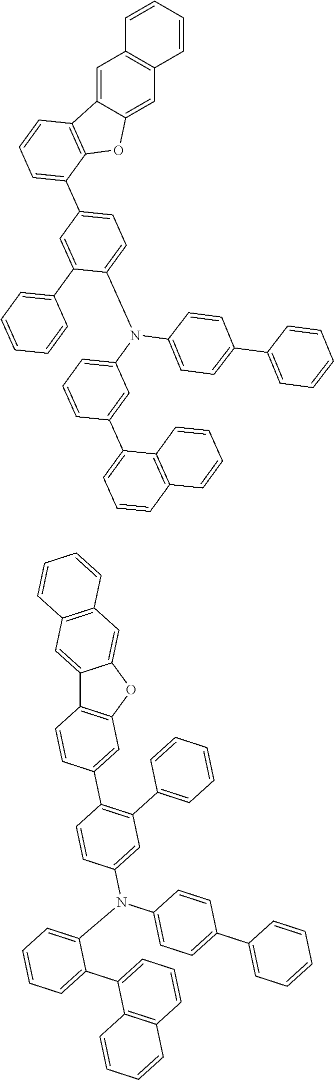 [SMILES: c1ccc(-c2ccc(N(c3cccc(-c4cccc5ccccc45)c3)c3ccc(-c4cccc5c4oc4cc6ccccc6cc45)cc3-c3ccccc3)cc2)cc1 ; c1ccc(-c2ccc(N(c3ccc(-c4ccc5c(c4)oc4cc6ccccc6cc45)c(-c4ccccc4)c3)c3ccccc3-c3cccc4ccccc34)cc2)cc1] 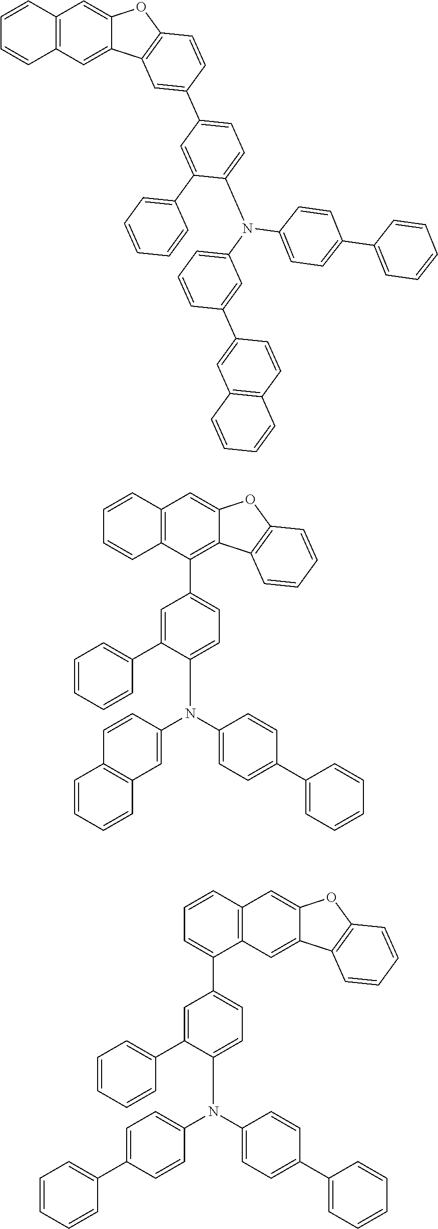 [SMILES: c1ccc(-c2ccc(N(c3cccc(-c4ccc5ccccc5c4)c3)c3ccc(-c4ccc5oc6cc7ccccc7cc6c5c4)cc3-c3ccccc3)cc2)cc1 ; c1ccc(-c2ccc(N(c3ccc4ccccc4c3)c3ccc(-c4c5ccccc5cc5oc6ccccc6c45)cc3-c3ccccc3)cc2)cc1 ; c1ccc(-c2ccc(N(c3ccc(-c4ccccc4)cc3)c3ccc(-c4cccc5cc6oc7ccccc7c6cc45)cc3-c3ccccc3)cc2)cc1] 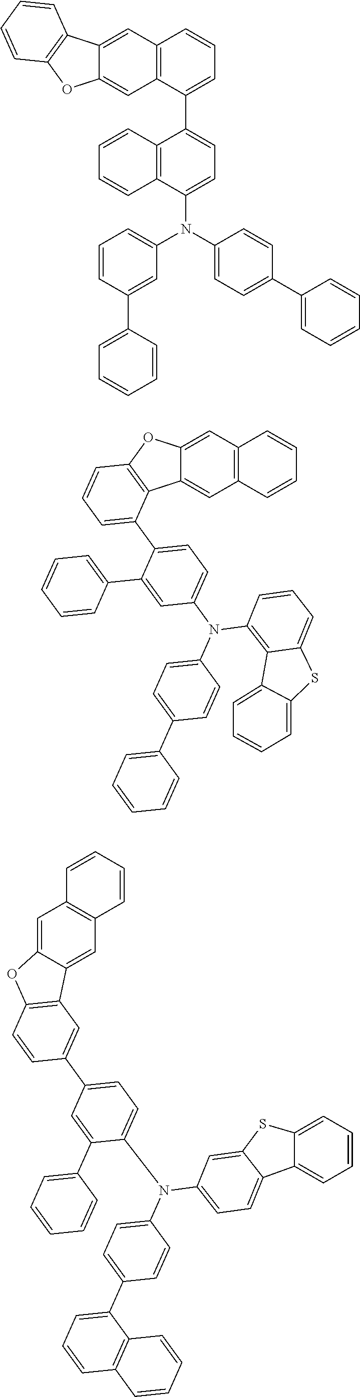 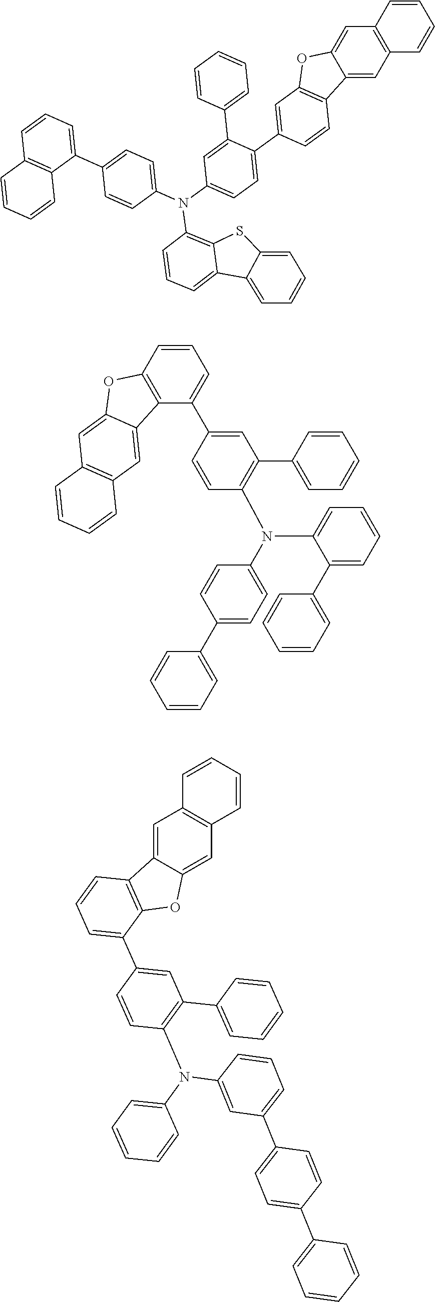 [SMILES: c1ccc(-c2cc(N(c3ccc(-c4cccc5ccccc45)cc3)c3cccc4c3sc3ccccc34)ccc2-c2ccc3c(c2)oc2cc4ccccc4cc23)cc1 ; c1ccc(-c2ccc(N(c3ccccc3-c3ccccc3)c3ccc(-c4cccc5oc6cc7ccccc7cc6c45)cc3-c3ccccc3)cc2)cc1 ; c1ccc(-c2ccc(-c3cccc(N(c4ccccc4)c4ccc(-c5cccc6c5oc5cc7ccccc7cc56)cc4-c4ccccc4)c3)cc2)cc1] 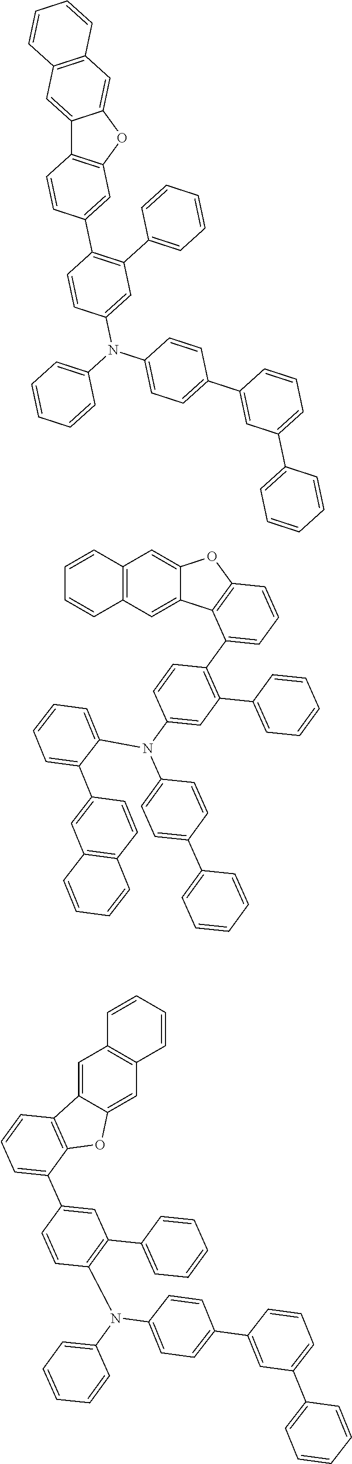 [SMILES: c1ccc(-c2cccc(-c3ccc(N(c4ccccc4)c4ccc(-c5ccc6c(c5)oc5cc7ccccc7cc56)c(-c5ccccc5)c4)cc3)c2)cc1 ; c1ccc(-c2ccc(N(c3ccc(-c4cccc5oc6cc7ccccc7cc6c45)c(-c4ccccc4)c3)c3ccccc3-c3ccc4ccccc4c3)cc2)cc1 ; c1ccc(-c2cccc(-c3ccc(N(c4ccccc4)c4ccc(-c5cccc6c5oc5cc7ccccc7cc56)cc4-c4ccccc4)cc3)c2)cc1] 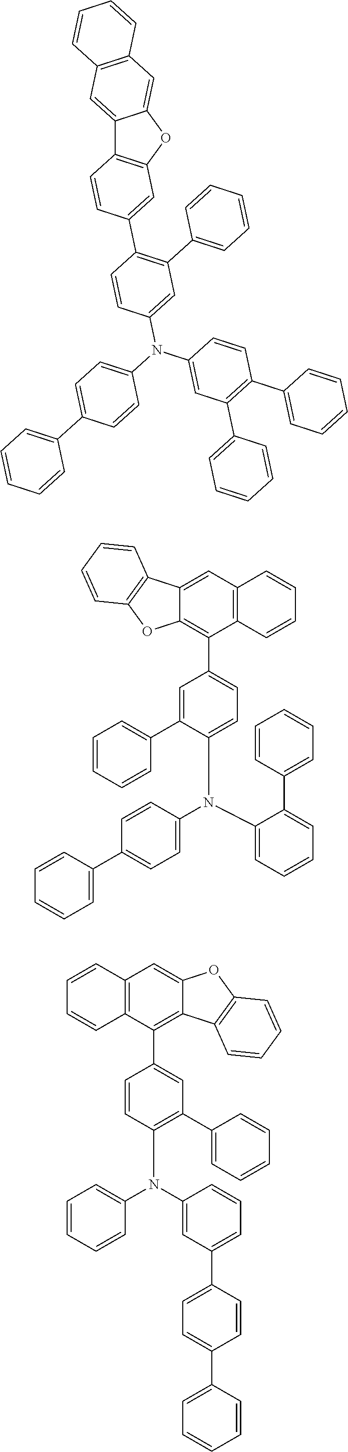 [SMILES: c1ccc(-c2ccc(N(c3ccc(-c4ccccc4)c(-c4ccccc4)c3)c3ccc(-c4ccc5c(c4)oc4cc6ccccc6cc45)c(-c4ccccc4)c3)cc2)cc1 ; c1ccc(-c2ccc(N(c3ccccc3-c3ccccc3)c3ccc(-c4c5ccccc5cc5c4oc4ccccc45)cc3-c3ccccc3)cc2)cc1 ; c1ccc(-c2ccc(-c3cccc(N(c4ccccc4)c4ccc(-c5c6ccccc6cc6oc7ccccc7c56)cc4-c4ccccc4)c3)cc2)cc1] 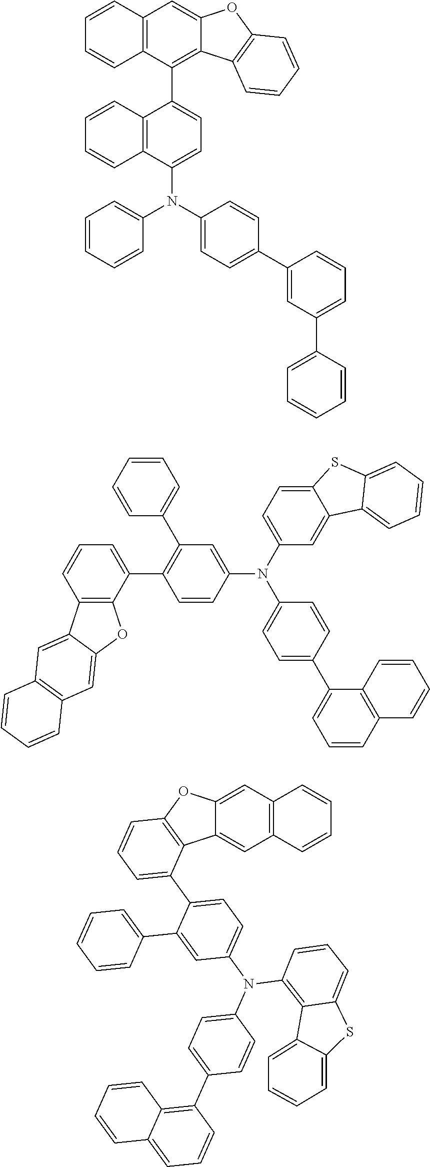 [SMILES: c1ccc(-c2cccc(-c3ccc(N(c4ccccc4)c4ccc(-c5c6ccccc6cc6oc7ccccc7c56)c5ccccc45)cc3)c2)cc1 ; c1ccc(-c2cc(N(c3ccc(-c4cccc5ccccc45)cc3)c3ccc4sc5ccccc5c4c3)ccc2-c2cccc3c2oc2cc4ccccc4cc23)cc1 ; c1ccc(-c2cc(N(c3ccc(-c4cccc5ccccc45)cc3)c3cccc4sc5ccccc5c34)ccc2-c2cccc3oc4cc5ccccc5cc4c23)cc1] 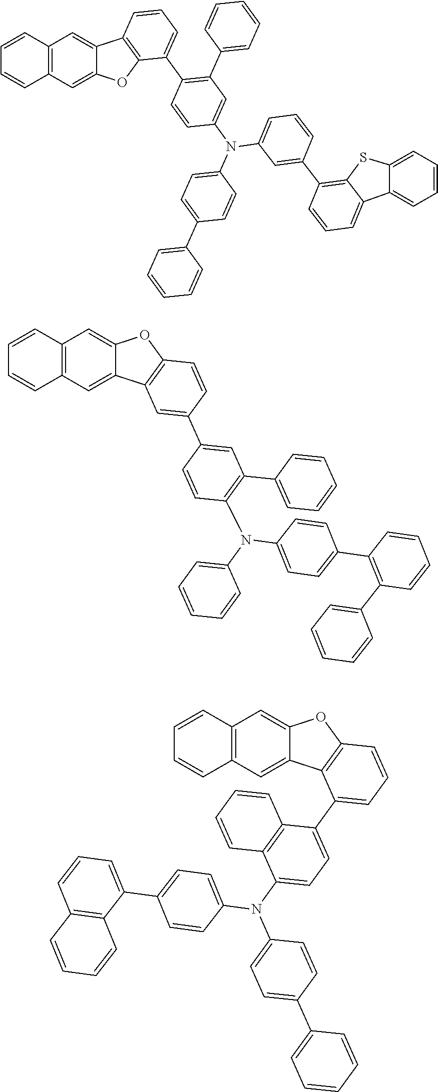 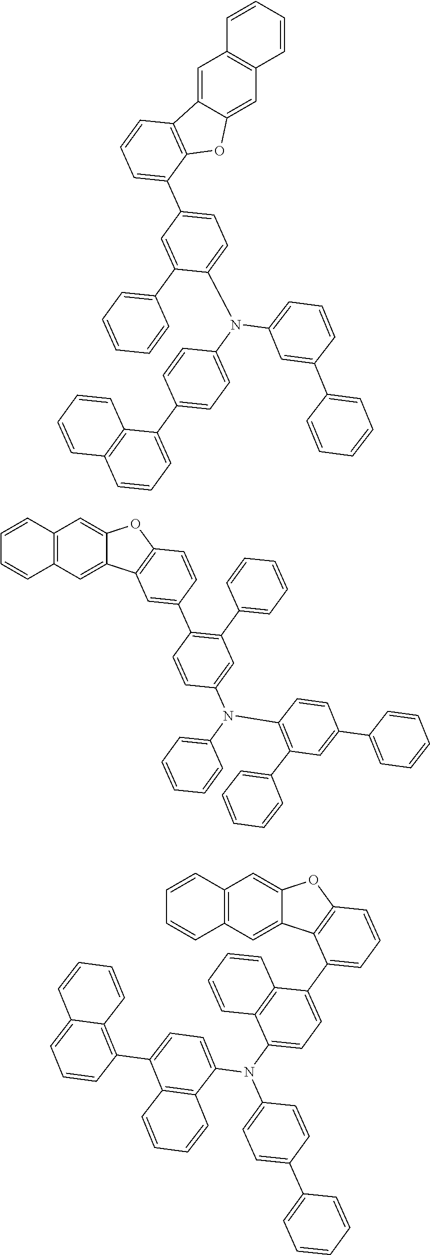 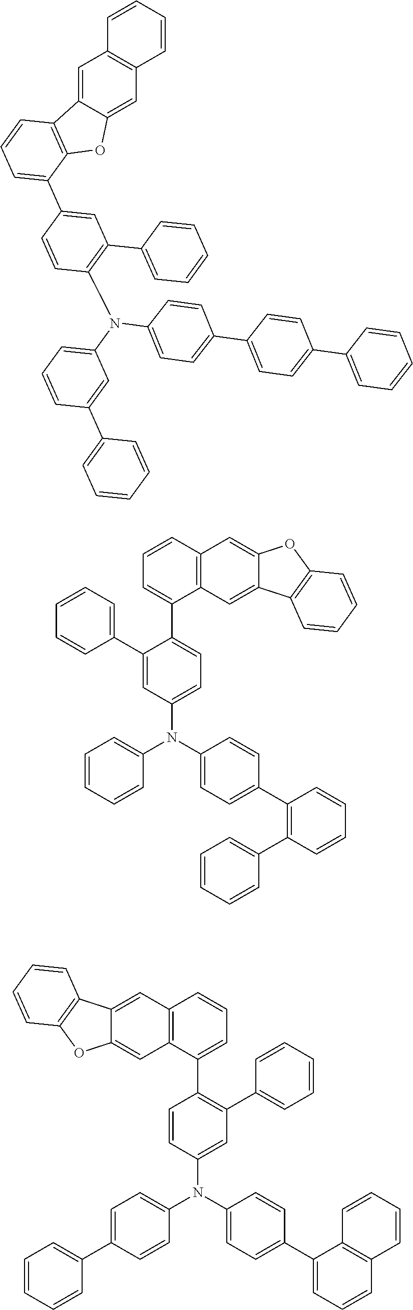 [SMILES: c1ccc(-c2ccc(-c3ccc(N(c4cccc(-c5ccccc5)c4)c4ccc(-c5cccc6c5oc5cc7ccccc7cc56)cc4-c4ccccc4)cc3)cc2)cc1 ; c1ccc(-c2ccccc2-c2ccc(N(c3ccccc3)c3ccc(-c4cccc5cc6oc7ccccc7c6cc45)c(-c4ccccc4)c3)cc2)cc1 ; c1ccc(-c2ccc(N(c3ccc(-c4cccc5ccccc45)cc3)c3ccc(-c4cccc5cc6c(cc45)oc4ccccc46)c(-c4ccccc4)c3)cc2)cc1] 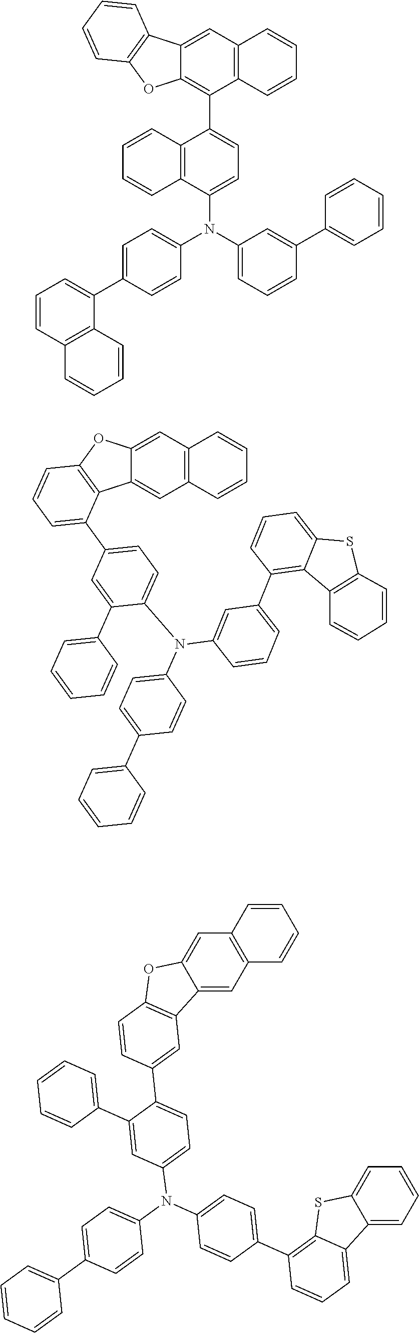 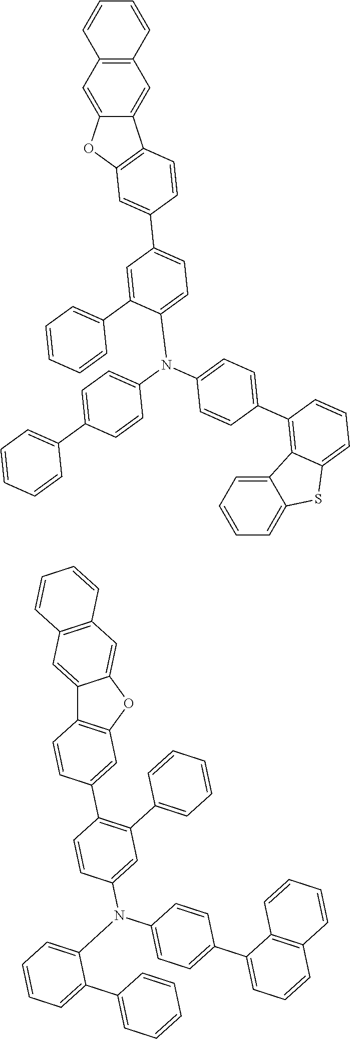 [SMILES: c1ccc(-c2ccc(N(c3ccc(-c4cccc5sc6ccccc6c45)cc3)c3ccc(-c4ccc5c(c4)oc4cc6ccccc6cc45)cc3-c3ccccc3)cc2)cc1 ; c1ccc(-c2cc(N(c3ccc(-c4cccc5ccccc45)cc3)c3ccccc3-c3ccccc3)ccc2-c2ccc3c(c2)oc2cc4ccccc4cc23)cc1] 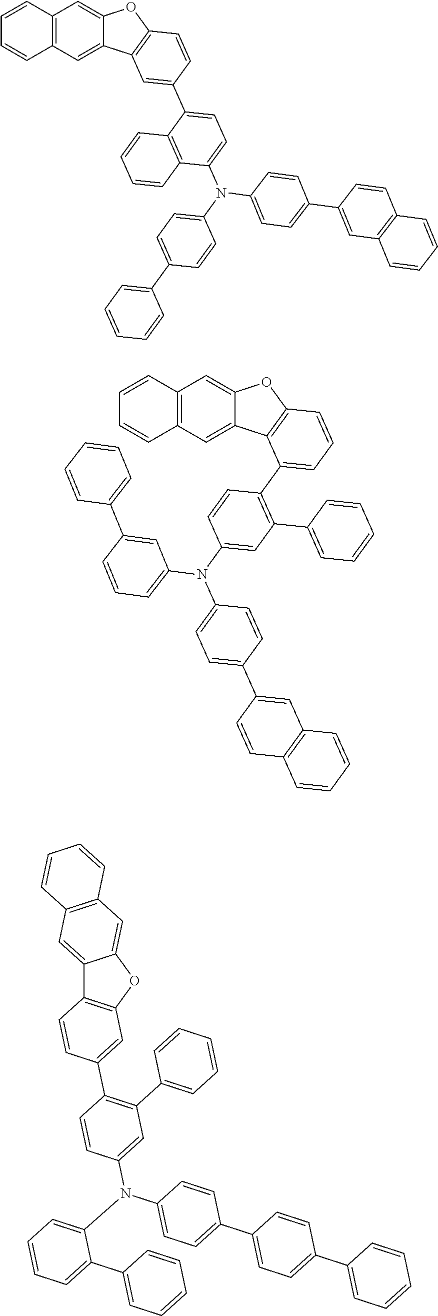 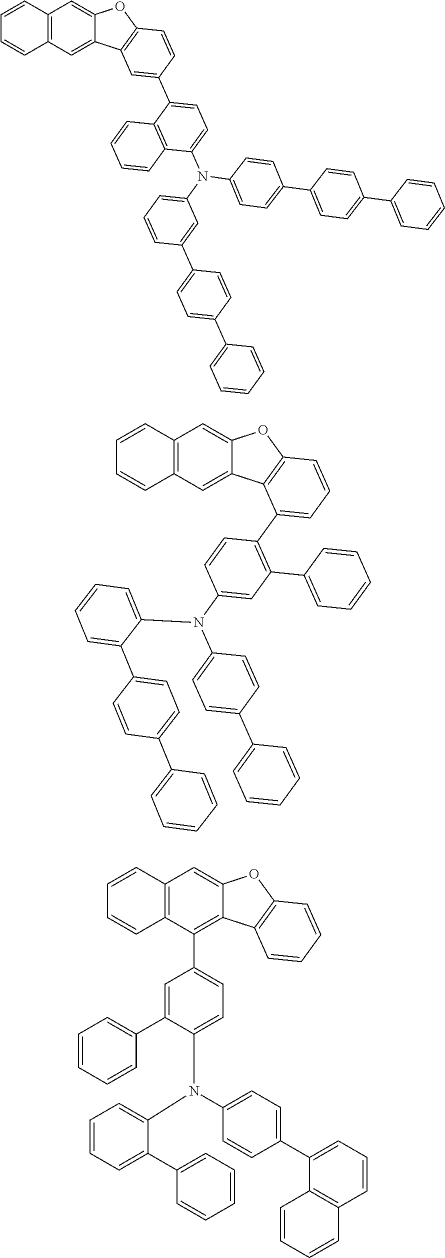 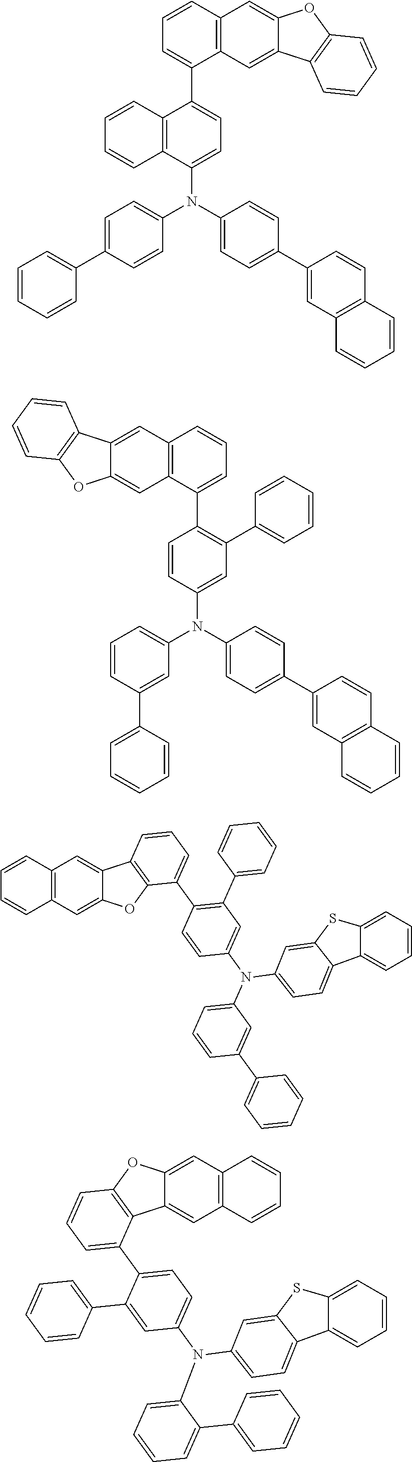 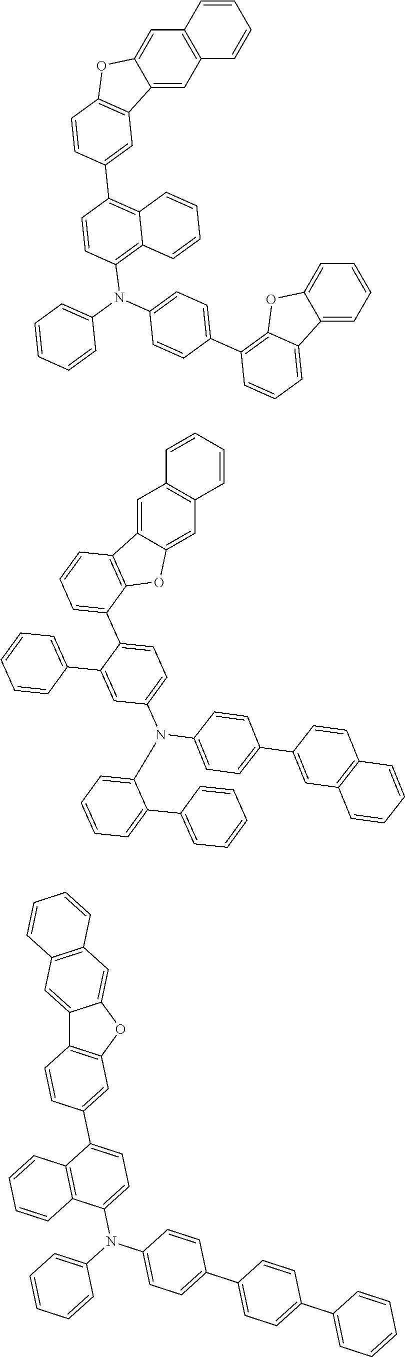 [SMILES: c1ccc(N(c2ccc(-c3cccc4c3oc3ccccc34)cc2)c2ccc(-c3ccc4oc5cc6ccccc6cc5c4c3)c3ccccc23)cc1 ; c1ccc(-c2cc(N(c3ccc(-c4ccc5ccccc5c4)cc3)c3ccccc3-c3ccccc3)ccc2-c2cccc3c2oc2cc4ccccc4cc23)cc1 ; c1ccc(-c2ccc(-c3ccc(N(c4ccccc4)c4ccc(-c5ccc6c(c5)oc5cc7ccccc7cc56)c5ccccc45)cc3)cc2)cc1] 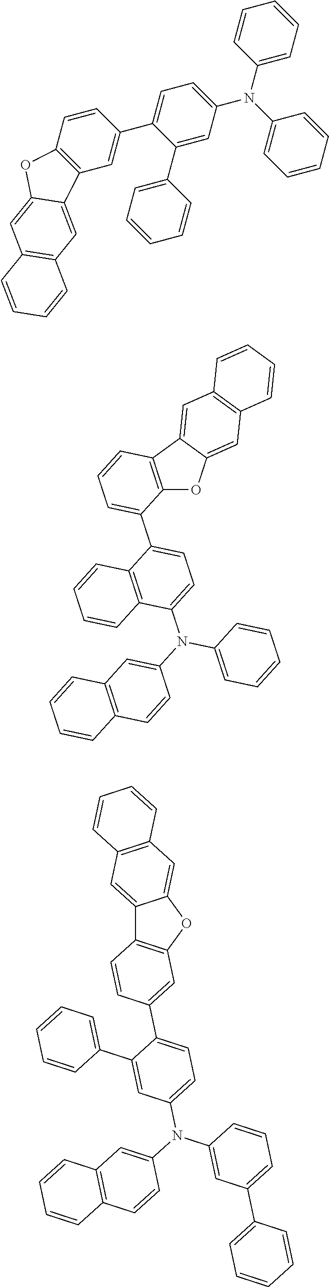 [SMILES: c1ccc(-c2cc(N(c3ccccc3)c3ccccc3)ccc2-c2ccc3oc4cc5ccccc5cc4c3c2)cc1 ; c1ccc(N(c2ccc3ccccc3c2)c2ccc(-c3cccc4c3oc3cc5ccccc5cc34)c3ccccc23)cc1 ; c1ccc(-c2cccc(N(c3ccc(-c4ccc5c(c4)oc4cc6ccccc6cc45)c(-c4ccccc4)c3)c3ccc4ccccc4c3)c2)cc1] 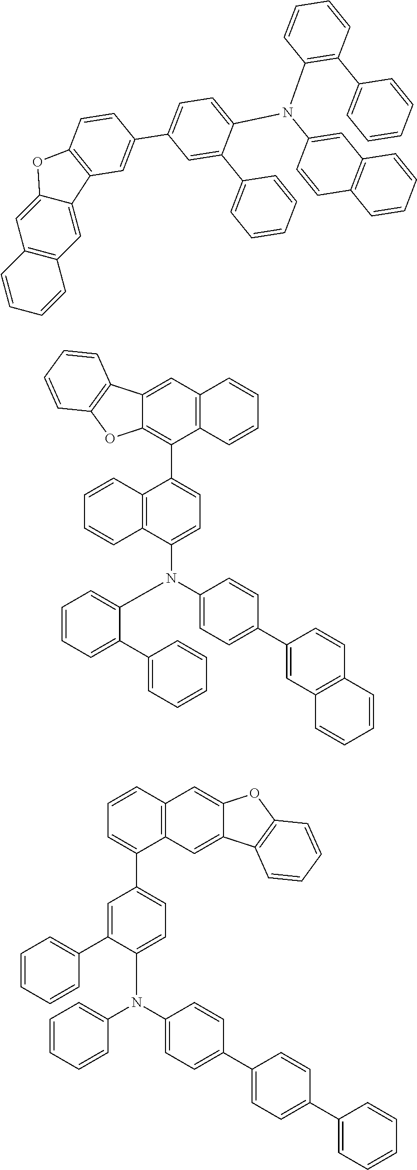 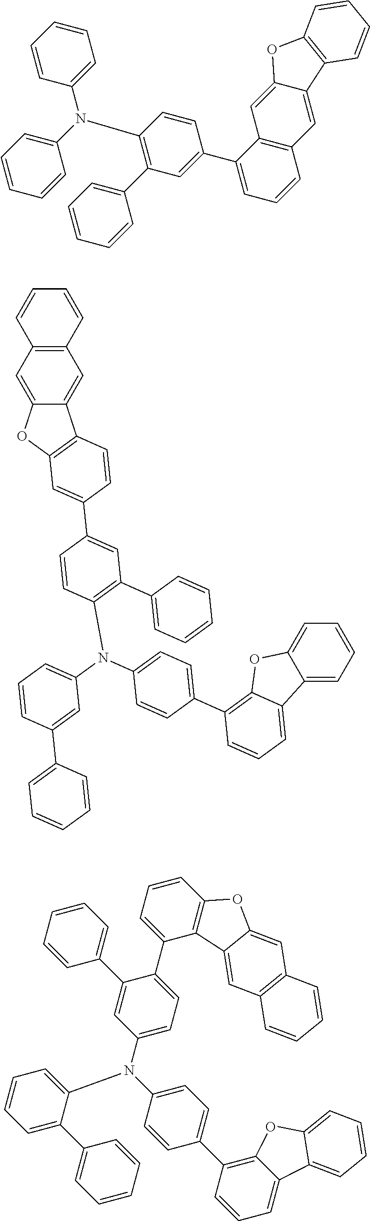 [SMILES: c1ccc(-c2cc(-c3cccc4cc5c(cc34)oc3ccccc35)ccc2N(c2ccccc2)c2ccccc2)cc1 ; c1ccc(-c2cccc(N(c3ccc(-c4cccc5c4oc4ccccc45)cc3)c3ccc(-c4ccc5c(c4)oc4cc6ccccc6cc45)cc3-c3ccccc3)c2)cc1 ; c1ccc(-c2cc(N(c3ccc(-c4cccc5c4oc4ccccc45)cc3)c3ccccc3-c3ccccc3)ccc2-c2cccc3oc4cc5ccccc5cc4c23)cc1] 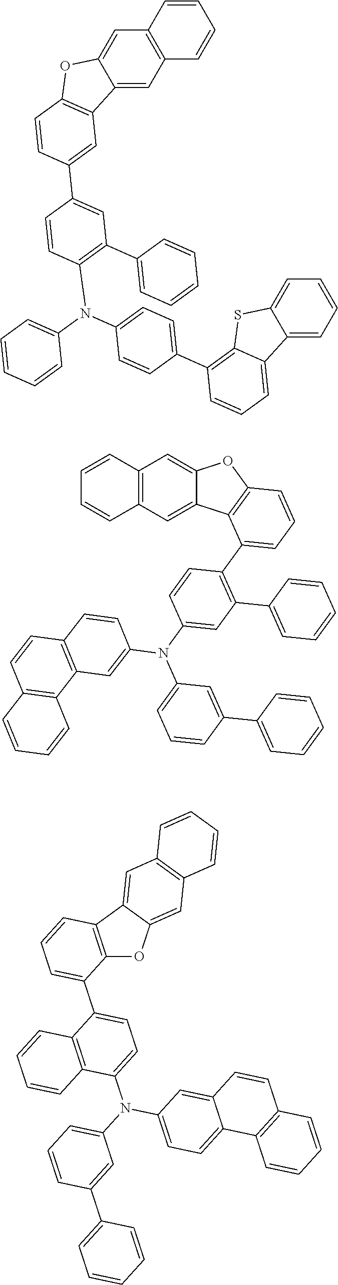 [SMILES: c1ccc(-c2cc(-c3ccc4oc5cc6ccccc6cc5c4c3)ccc2N(c2ccccc2)c2ccc(-c3cccc4c3sc3ccccc34)cc2)cc1 ; c1ccc(-c2cccc(N(c3ccc(-c4cccc5oc6cc7ccccc7cc6c45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4cccc5c4oc4cc6ccccc6cc45)c4ccccc34)c2)cc1] 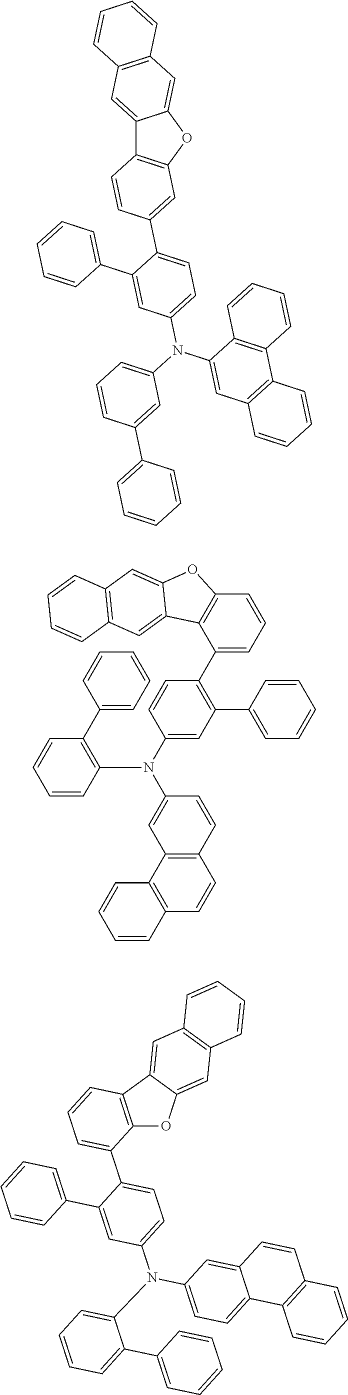 [SMILES: c1ccc(-c2cccc(N(c3ccc(-c4ccc5c(c4)oc4cc6ccccc6cc45)c(-c4ccccc4)c3)c3cc4ccccc4c4ccccc34)c2)cc1 ; c1ccc(-c2cc(N(c3ccc4ccc5ccccc5c4c3)c3ccccc3-c3ccccc3)ccc2-c2cccc3oc4cc5ccccc5cc4c23)cc1 ; c1ccc(-c2cc(N(c3ccc4c(ccc5ccccc54)c3)c3ccccc3-c3ccccc3)ccc2-c2cccc3c2oc2cc4ccccc4cc23)cc1] 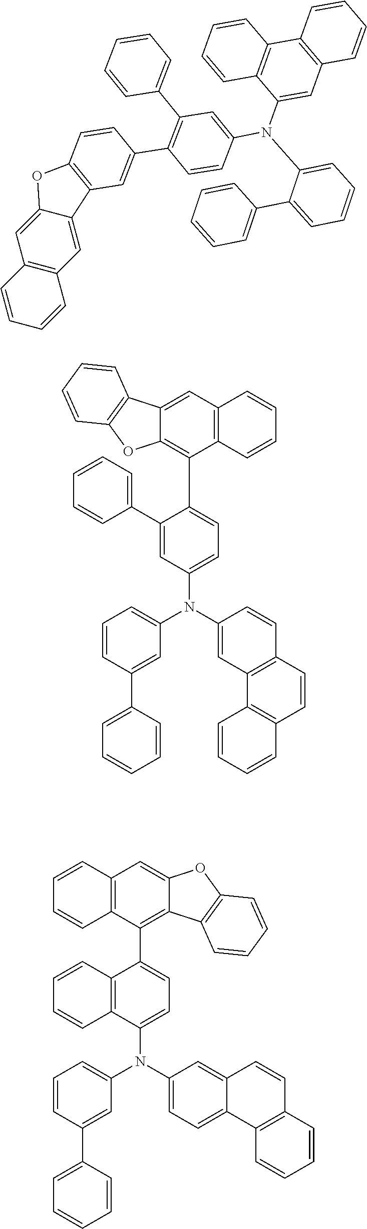 [SMILES: c1ccc(-c2cc(N(c3ccccc3-c3ccccc3)c3cc4ccccc4c4ccccc34)ccc2-c2ccc3oc4cc5ccccc5cc4c3c2)cc1 ; c1ccc(-c2cccc(N(c3ccc(-c4c5ccccc5cc5c4oc4ccccc45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4c5ccccc5cc5oc6ccccc6c45)c4ccccc34)c2)cc1] 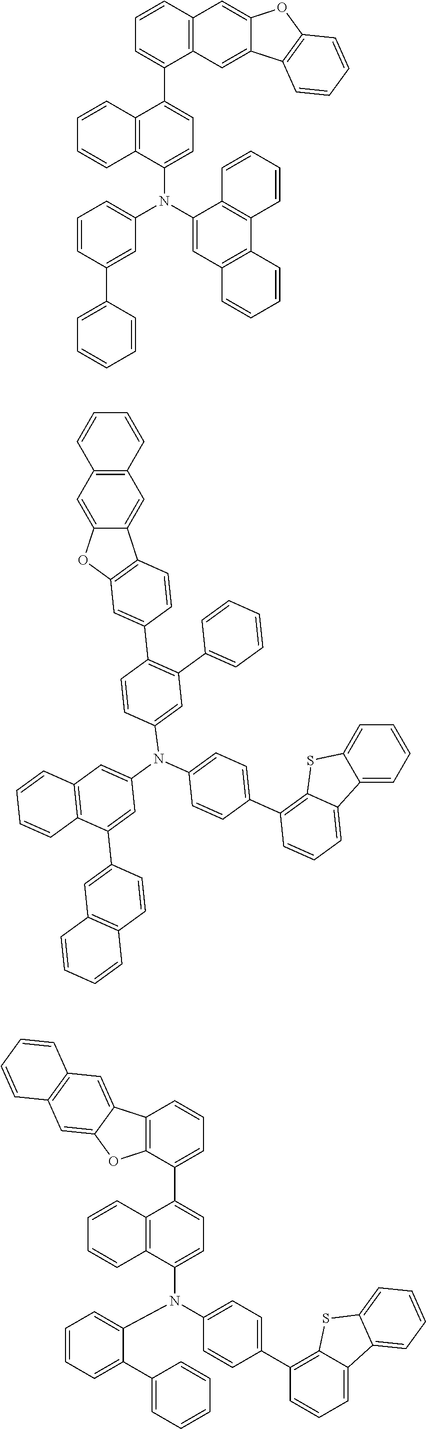 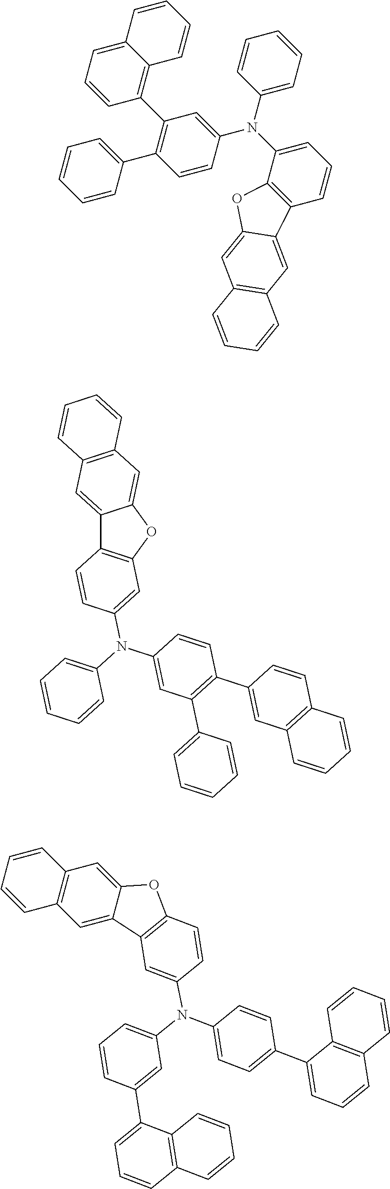 [SMILES: c1ccc(-c2ccc(N(c3ccccc3)c3cccc4c3oc3cc5ccccc5cc34)cc2-c2cccc3ccccc23)cc1 ; c1ccc(-c2cc(N(c3ccccc3)c3ccc4c(c3)oc3cc5ccccc5cc34)ccc2-c2ccc3ccccc3c2)cc1 ; c1cc(-c2cccc3ccccc23)cc(N(c2ccc(-c3cccc4ccccc34)cc2)c2ccc3oc4cc5ccccc5cc4c3c2)c1] 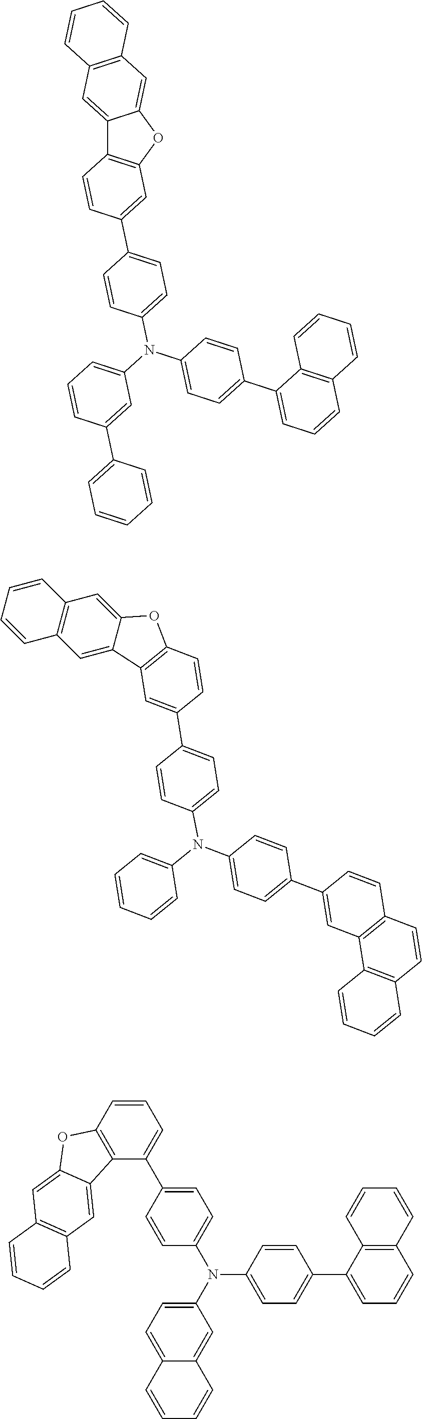 [SMILES: c1ccc(-c2cccc(N(c3ccc(-c4ccc5c(c4)oc4cc6ccccc6cc45)cc3)c3ccc(-c4cccc5ccccc45)cc3)c2)cc1 ; c1ccc(N(c2ccc(-c3ccc4ccc5ccccc5c4c3)cc2)c2ccc(-c3ccc4oc5cc6ccccc6cc5c4c3)cc2)cc1 ; c1ccc2cc(N(c3ccc(-c4cccc5ccccc45)cc3)c3ccc(-c4cccc5oc6cc7ccccc7cc6c45)cc3)ccc2c1] 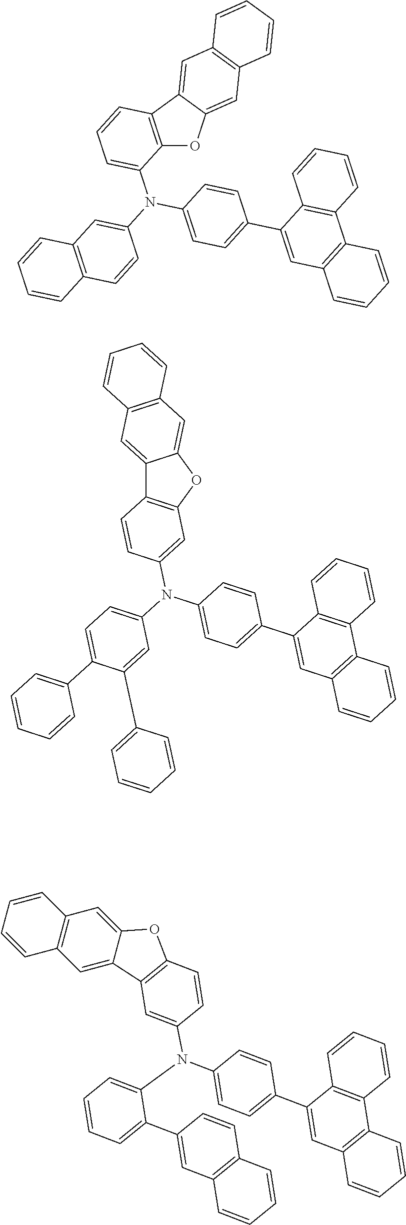 [SMILES: c1ccc2cc(N(c3ccc(-c4cc5ccccc5c5ccccc45)cc3)c3cccc4c3oc3cc5ccccc5cc34)ccc2c1 ; c1ccc(-c2ccc(N(c3ccc(-c4cc5ccccc5c5ccccc45)cc3)c3ccc4c(c3)oc3cc5ccccc5cc34)cc2-c2ccccc2)cc1 ; c1ccc(N(c2ccc(-c3cc4ccccc4c4ccccc34)cc2)c2ccc3oc4cc5ccccc5cc4c3c2)c(-c2ccc3ccccc3c2)c1] 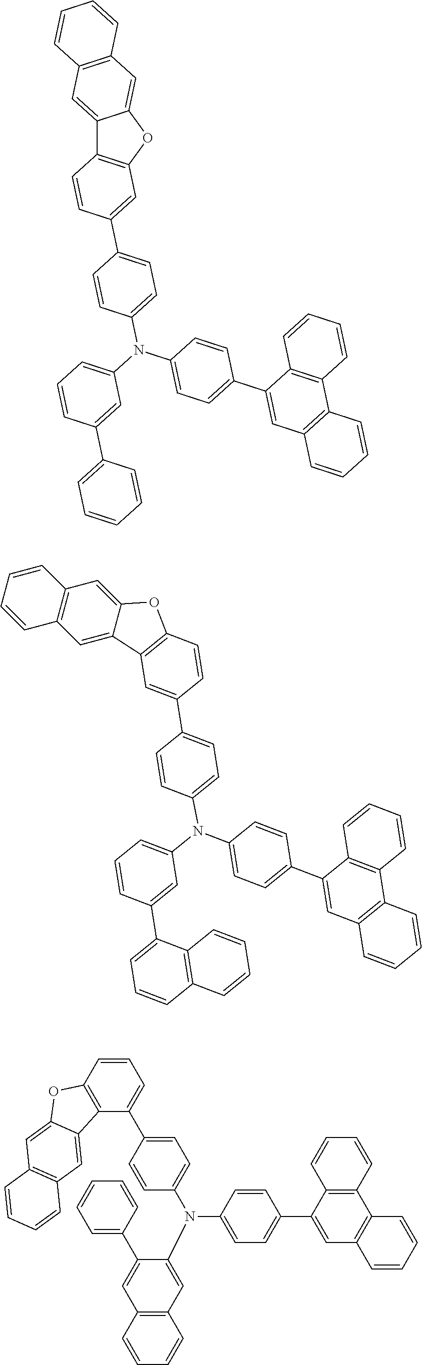 [SMILES: c1ccc(-c2cccc(N(c3ccc(-c4ccc5c(c4)oc4cc6ccccc6cc45)cc3)c3ccc(-c4cc5ccccc5c5ccccc45)cc3)c2)cc1 ; c1cc(-c2cccc3ccccc23)cc(N(c2ccc(-c3ccc4oc5cc6ccccc6cc5c4c3)cc2)c2ccc(-c3cc4ccccc4c4ccccc34)cc2)c1 ; c1ccc(-c2cc3ccccc3cc2N(c2ccc(-c3cc4ccccc4c4ccccc34)cc2)c2ccc(-c3cccc4oc5cc6ccccc6cc5c34)cc2)cc1] 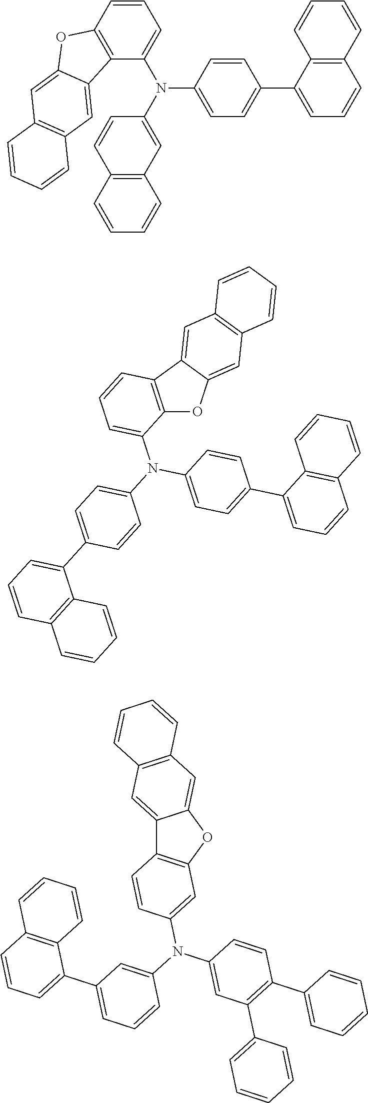 [SMILES: c1ccc2cc(N(c3ccc(-c4cccc5ccccc45)cc3)c3cccc4oc5cc6ccccc6cc5c34)ccc2c1 ; c1ccc2cc3c(cc2c1)oc1c(N(c2ccc(-c4cccc5ccccc45)cc2)c2ccc(-c4cccc5ccccc45)cc2)cccc13 ; c1ccc(-c2ccc(N(c3cccc(-c4cccc5ccccc45)c3)c3ccc4c(c3)oc3cc5ccccc5cc34)cc2-c2ccccc2)cc1] 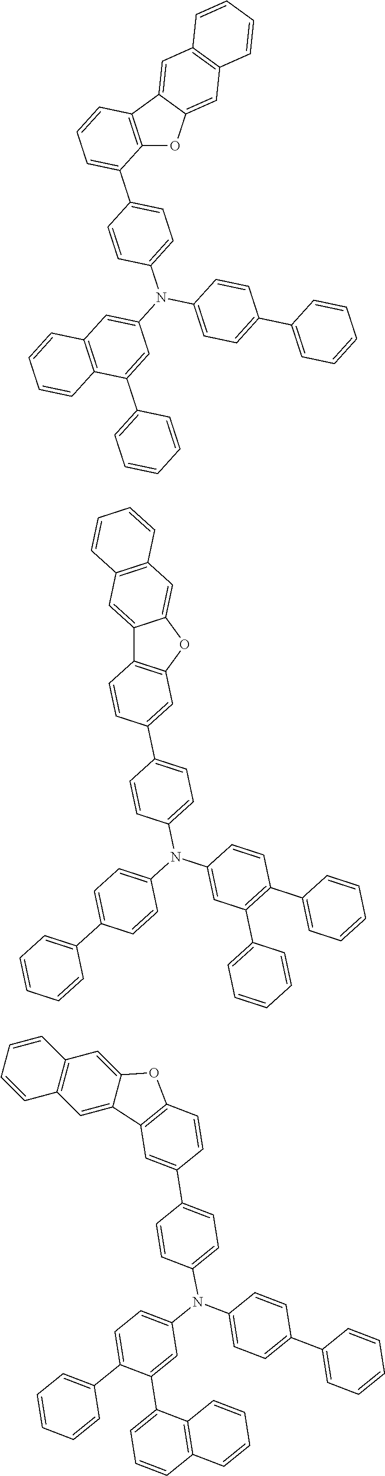 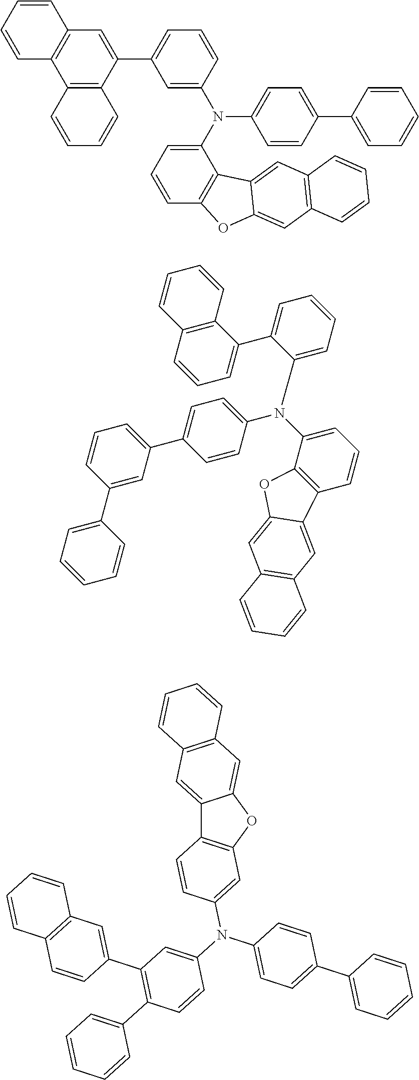 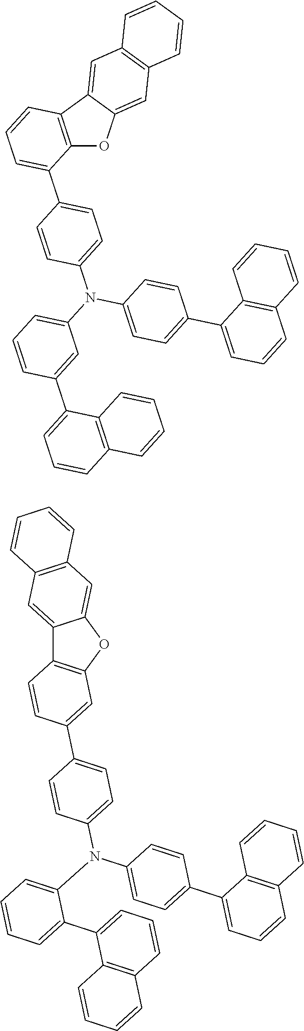 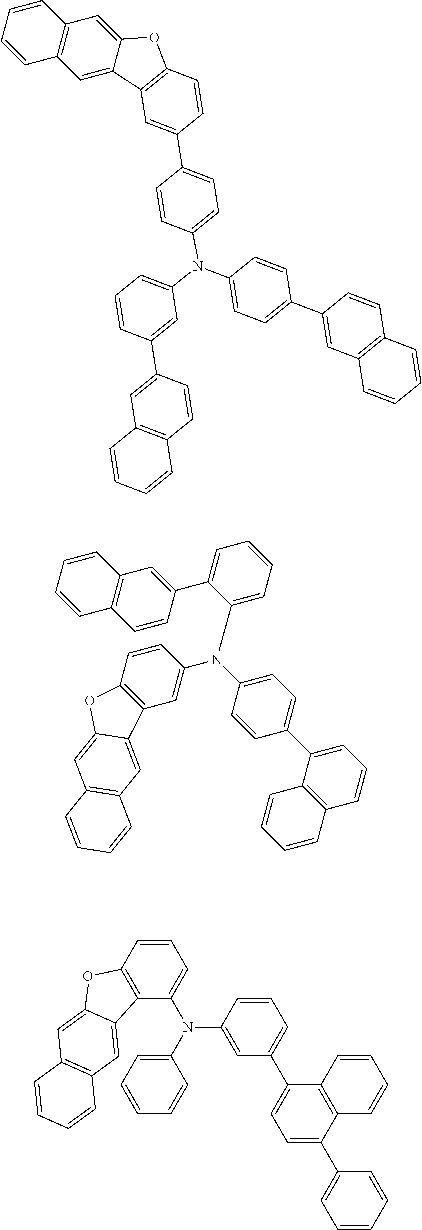 [SMILES: c1cc(-c2ccc3ccccc3c2)cc(N(c2ccc(-c3ccc4ccccc4c3)cc2)c2ccc(-c3ccc4oc5cc6ccccc6cc5c4c3)cc2)c1 ; c1ccc(N(c2ccc(-c3cccc4ccccc34)cc2)c2ccc3oc4cc5ccccc5cc4c3c2)c(-c2ccc3ccccc3c2)c1 ; c1ccc(-c2ccc(-c3cccc(N(c4ccccc4)c4cccc5oc6cc7ccccc7cc6c45)c3)c3ccccc23)cc1] 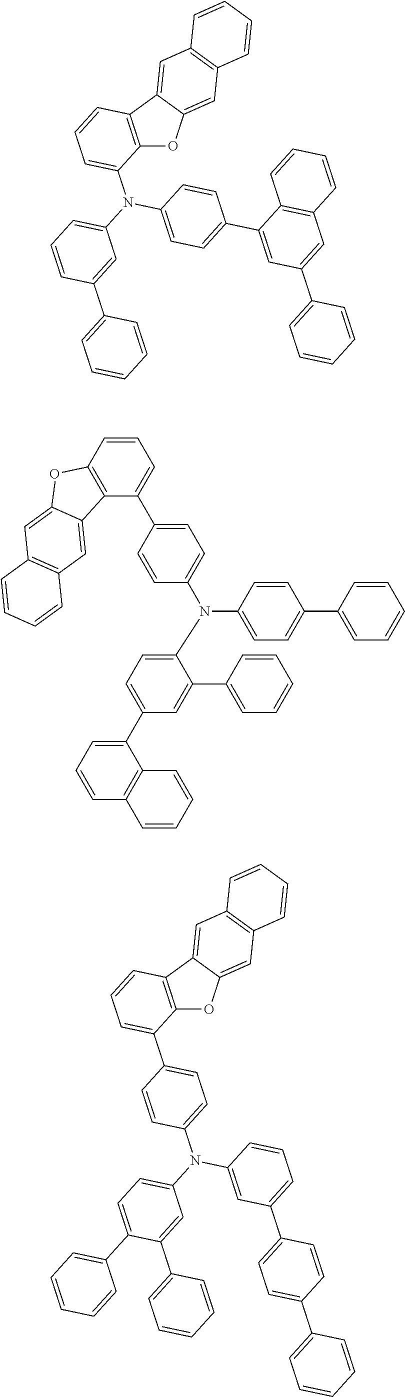 [SMILES: c1ccc(-c2cccc(N(c3ccc(-c4cc(-c5ccccc5)cc5ccccc45)cc3)c3cccc4c3oc3cc5ccccc5cc34)c2)cc1 ; c1ccc(-c2ccc(N(c3ccc(-c4cccc5oc6cc7ccccc7cc6c45)cc3)c3ccc(-c4cccc5ccccc45)cc3-c3ccccc3)cc2)cc1 ; c1ccc(-c2ccc(-c3cccc(N(c4ccc(-c5cccc6c5oc5cc7ccccc7cc56)cc4)c4ccc(-c5ccccc5)c(-c5ccccc5)c4)c3)cc2)cc1] 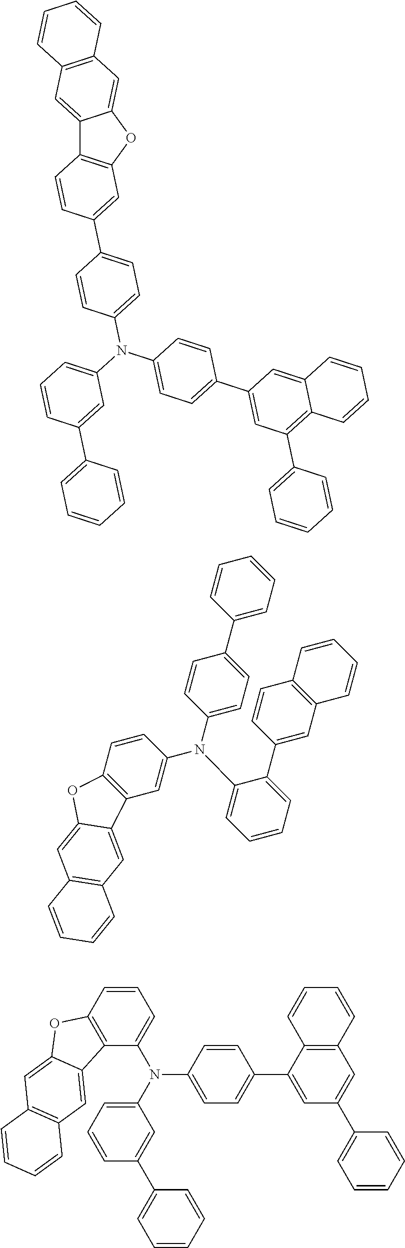 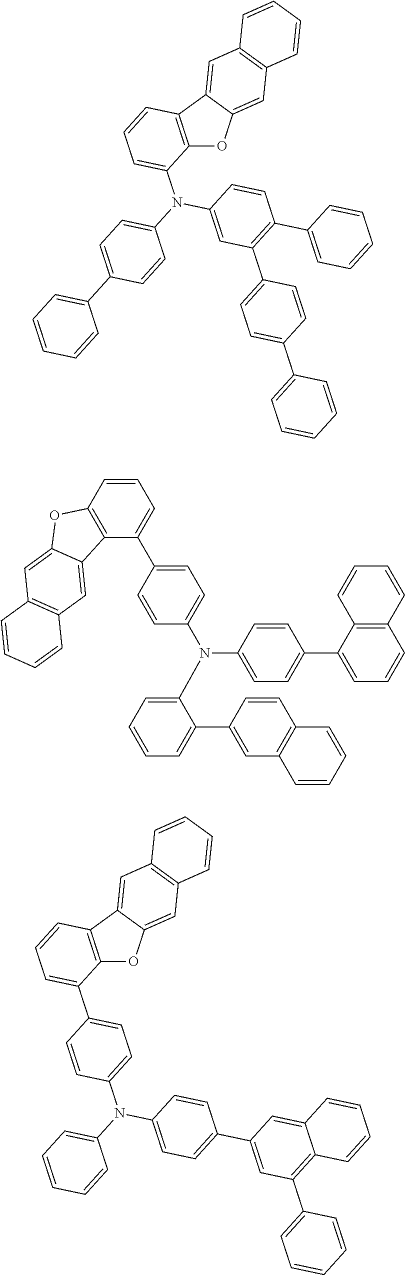 [SMILES: c1ccc(-c2ccc(-c3cc(N(c4ccc(-c5ccccc5)cc4)c4cccc5c4oc4cc6ccccc6cc45)ccc3-c3ccccc3)cc2)cc1 ; c1ccc(N(c2ccc(-c3cccc4ccccc34)cc2)c2ccc(-c3cccc4oc5cc6ccccc6cc5c34)cc2)c(-c2ccc3ccccc3c2)c1 ; c1ccc(-c2cc(-c3ccc(N(c4ccccc4)c4ccc(-c5cccc6c5oc5cc7ccccc7cc56)cc4)cc3)cc3ccccc23)cc1] 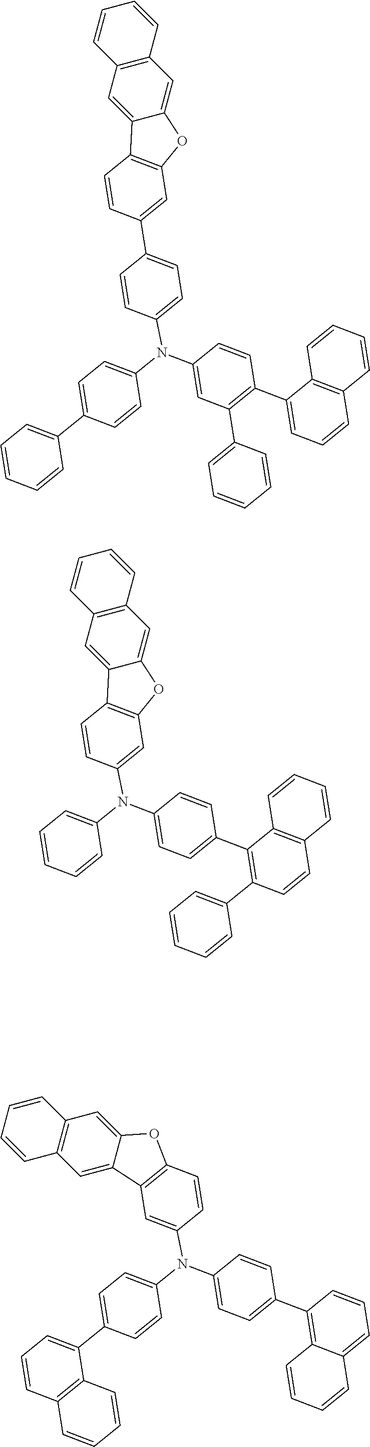 [SMILES: c1ccc(-c2ccc(N(c3ccc(-c4ccc5c(c4)oc4cc6ccccc6cc45)cc3)c3ccc(-c4cccc5ccccc45)c(-c4ccccc4)c3)cc2)cc1 ; c1ccc(-c2ccc3ccccc3c2-c2ccc(N(c3ccccc3)c3ccc4c(c3)oc3cc5ccccc5cc34)cc2)cc1 ; c1ccc2cc3c(cc2c1)oc1ccc(N(c2ccc(-c4cccc5ccccc45)cc2)c2ccc(-c4cccc5ccccc45)cc2)cc13] 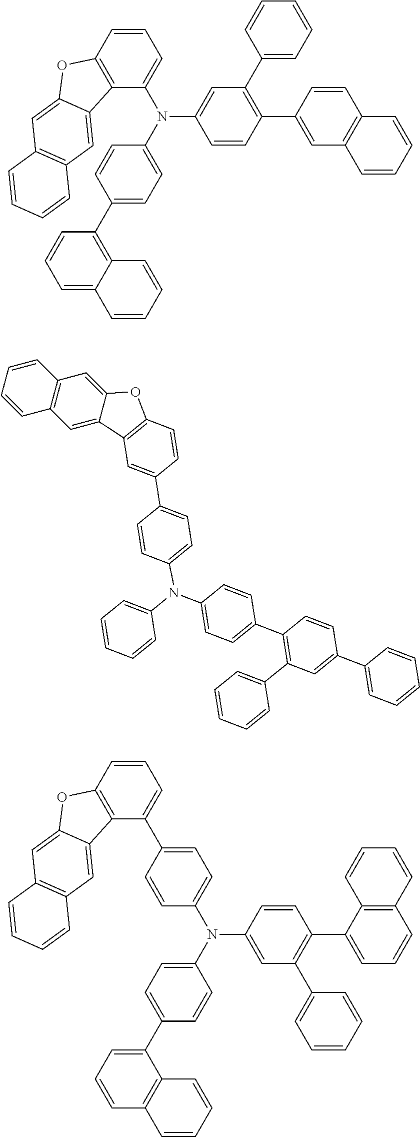 [SMILES: c1ccc(-c2cc(N(c3ccc(-c4cccc5ccccc45)cc3)c3cccc4oc5cc6ccccc6cc5c34)ccc2-c2ccc3ccccc3c2)cc1 ; c1ccc(-c2ccc(-c3ccc(N(c4ccccc4)c4ccc(-c5ccc6oc7cc8ccccc8cc7c6c5)cc4)cc3)c(-c3ccccc3)c2)cc1 ; c1ccc(-c2cc(N(c3ccc(-c4cccc5ccccc45)cc3)c3ccc(-c4cccc5oc6cc7ccccc7cc6c45)cc3)ccc2-c2cccc3ccccc23)cc1] 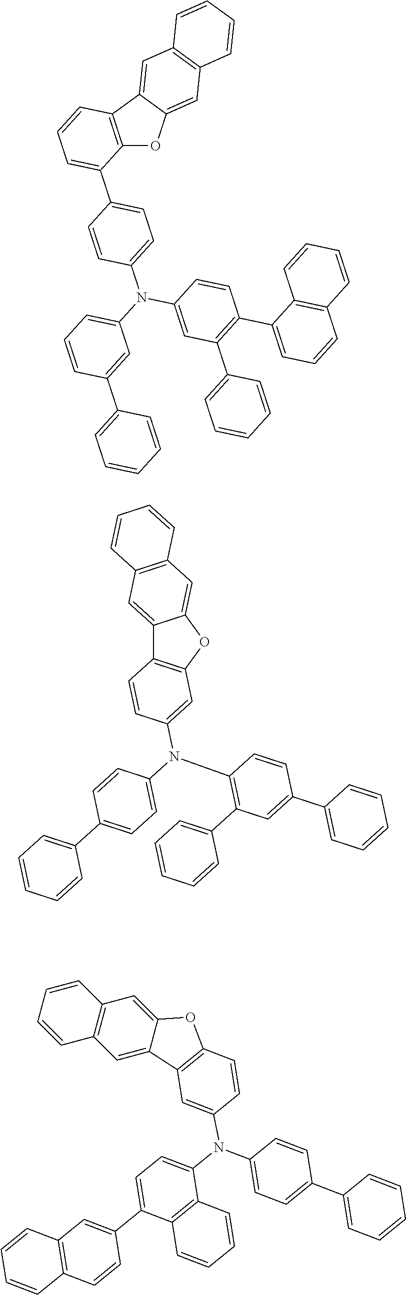 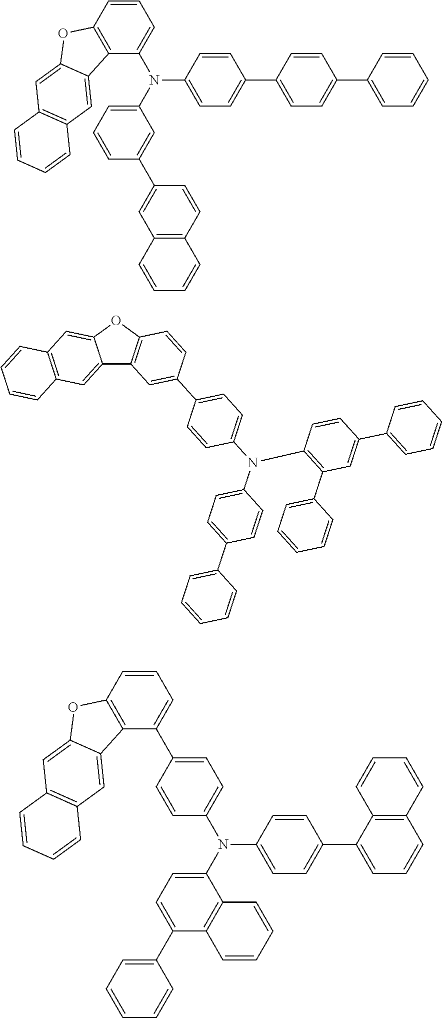 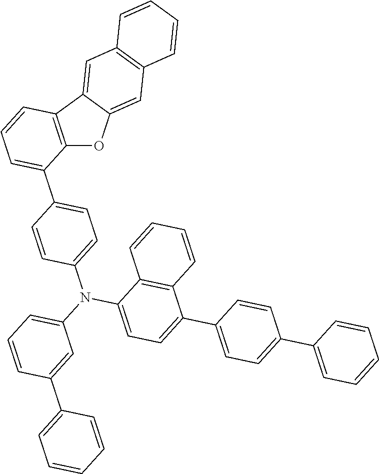 [SMILES: c1ccc(-c2ccc(-c3ccc(N(c4ccc(-c5cccc6c5oc5cc7ccccc7cc56)cc4)c4cccc(-c5ccccc5)c4)c4ccccc34)cc2)cc1] 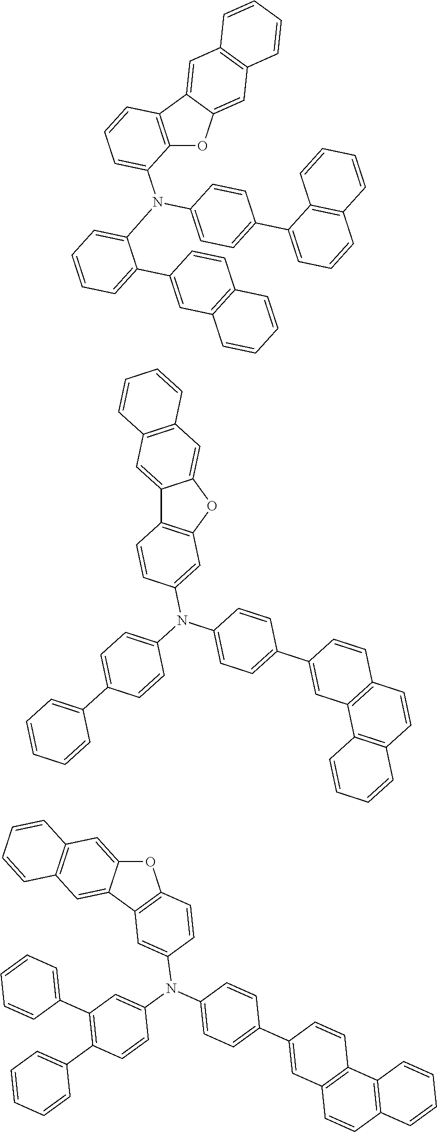 [SMILES: c1ccc(N(c2ccc(-c3cccc4ccccc34)cc2)c2cccc3c2oc2cc4ccccc4cc23)c(-c2ccc3ccccc3c2)c1 ; c1ccc(-c2ccc(N(c3ccc(-c4ccc5ccc6ccccc6c5c4)cc3)c3ccc4c(c3)oc3cc5ccccc5cc34)cc2)cc1 ; c1ccc(-c2ccc(N(c3ccc(-c4ccc5c(ccc6ccccc65)c4)cc3)c3ccc4oc5cc6ccccc6cc5c4c3)cc2-c2ccccc2)cc1] 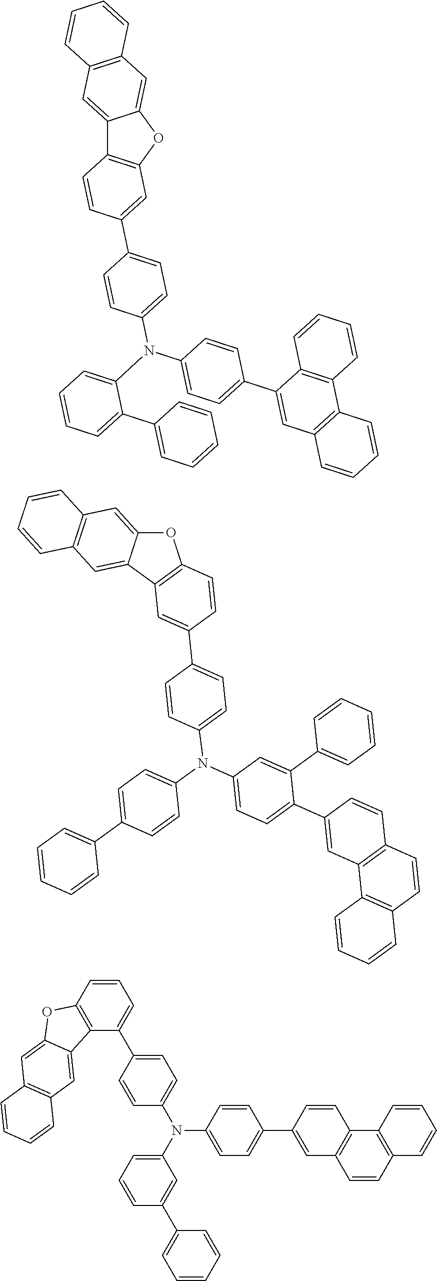 [SMILES: c1ccc(-c2ccccc2N(c2ccc(-c3ccc4c(c3)oc3cc5ccccc5cc34)cc2)c2ccc(-c3cc4ccccc4c4ccccc34)cc2)cc1 ; c1ccc(-c2ccc(N(c3ccc(-c4ccc5oc6cc7ccccc7cc6c5c4)cc3)c3ccc(-c4ccc5ccc6ccccc6c5c4)c(-c4ccccc4)c3)cc2)cc1 ; c1ccc(-c2cccc(N(c3ccc(-c4ccc5c(ccc6ccccc65)c4)cc3)c3ccc(-c4cccc5oc6cc7ccccc7cc6c45)cc3)c2)cc1] 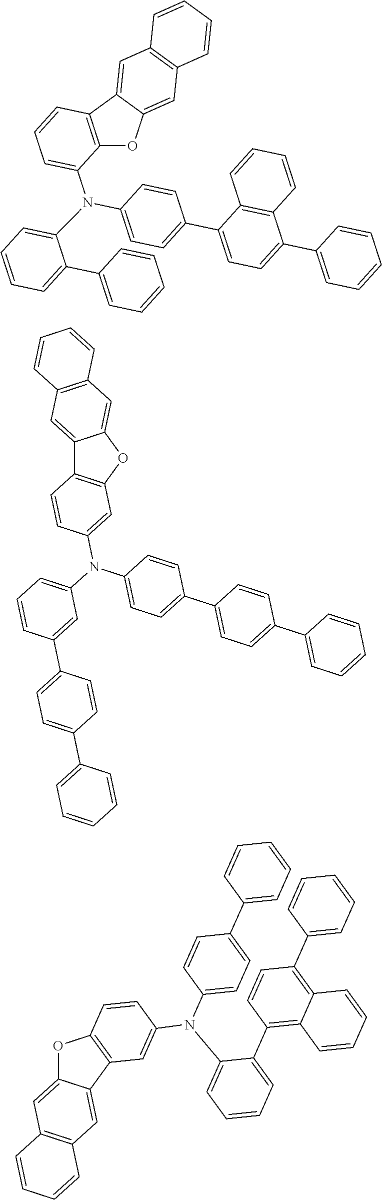 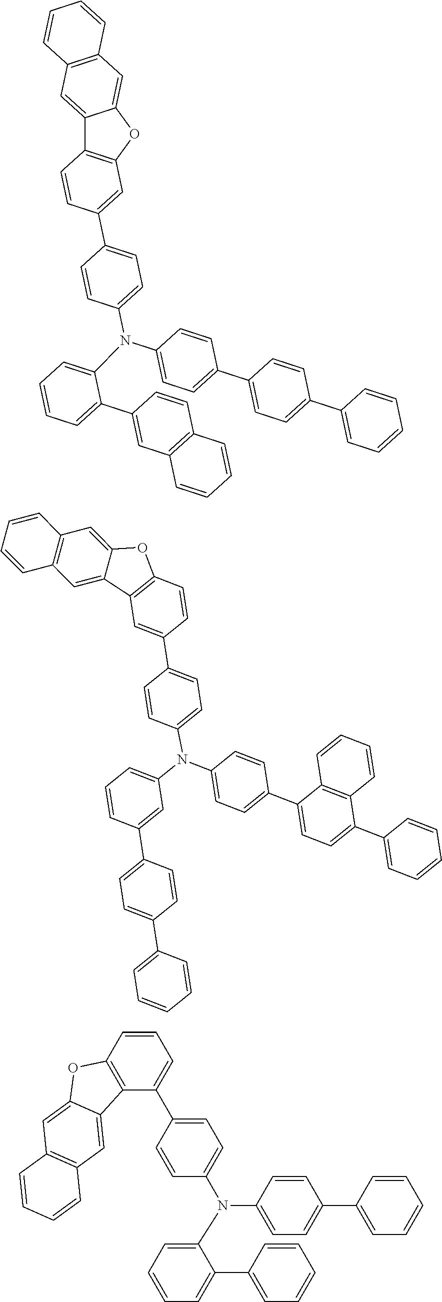 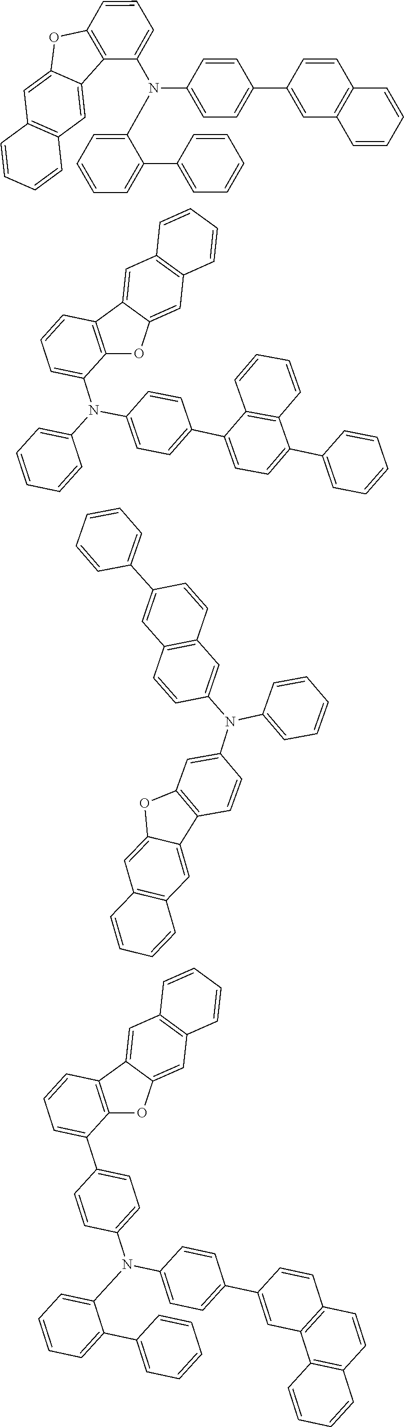 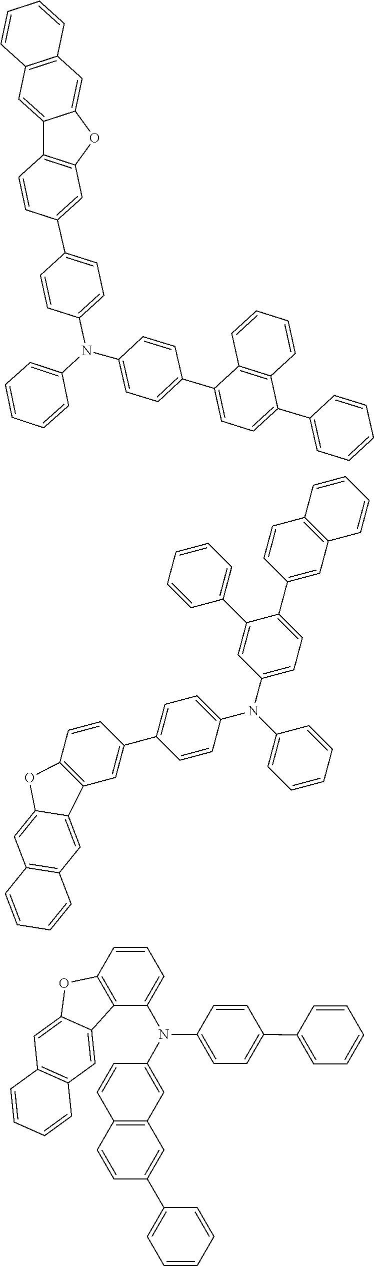 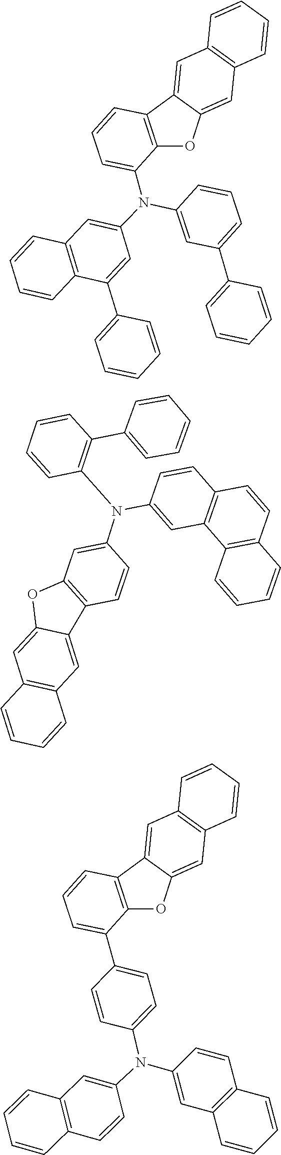 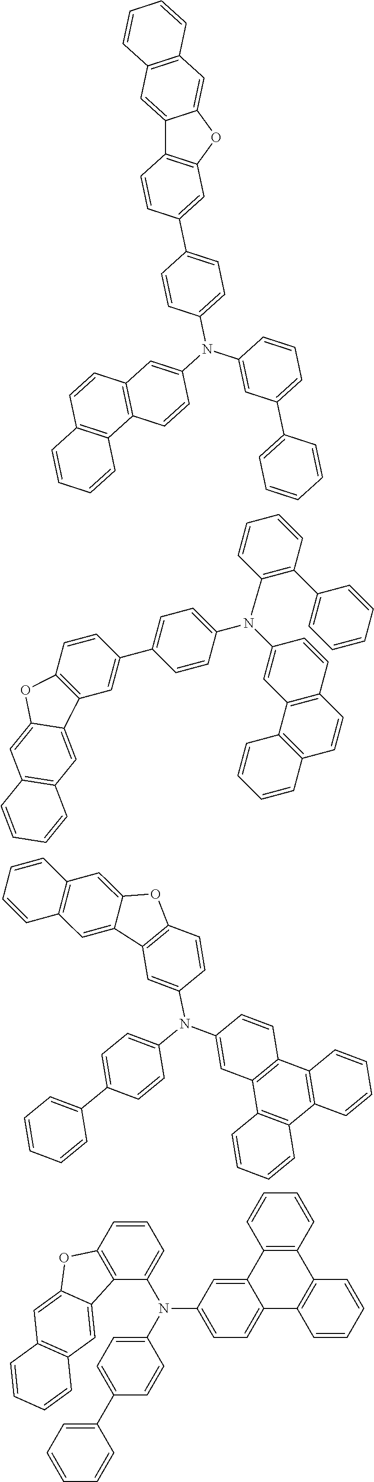 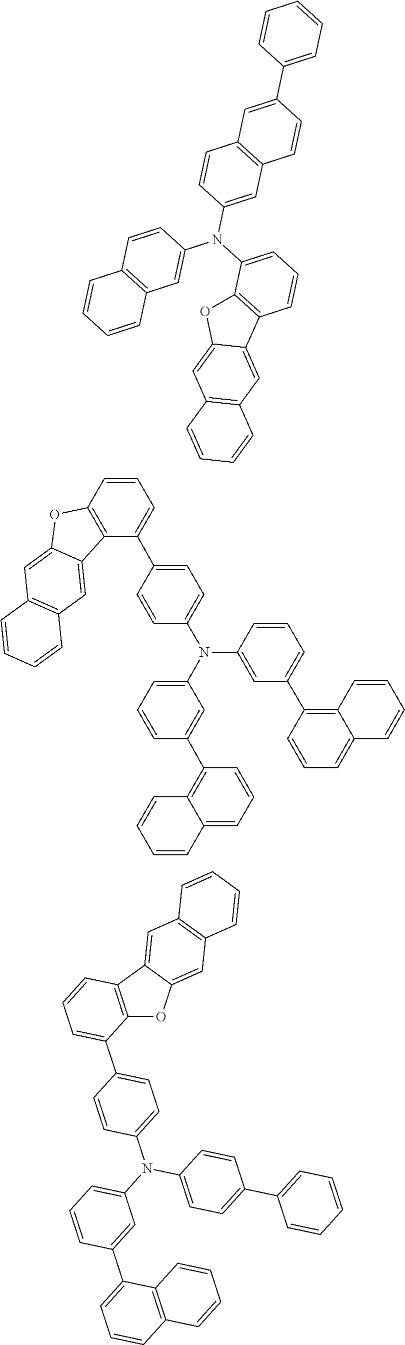 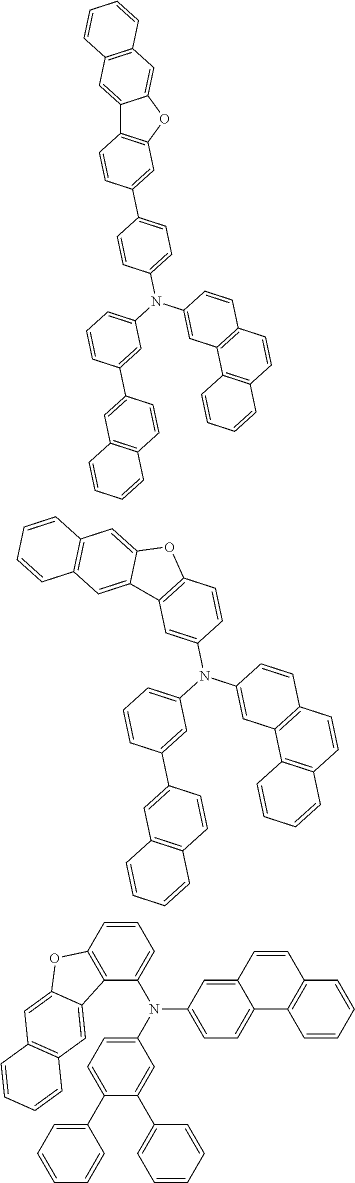 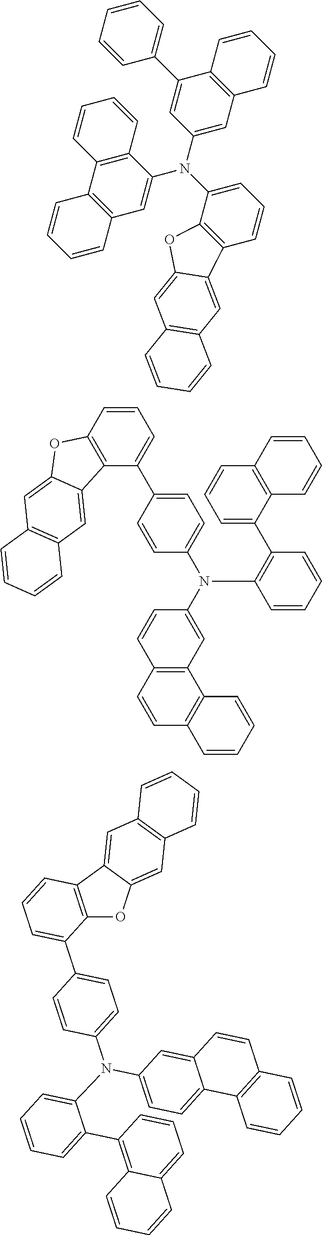 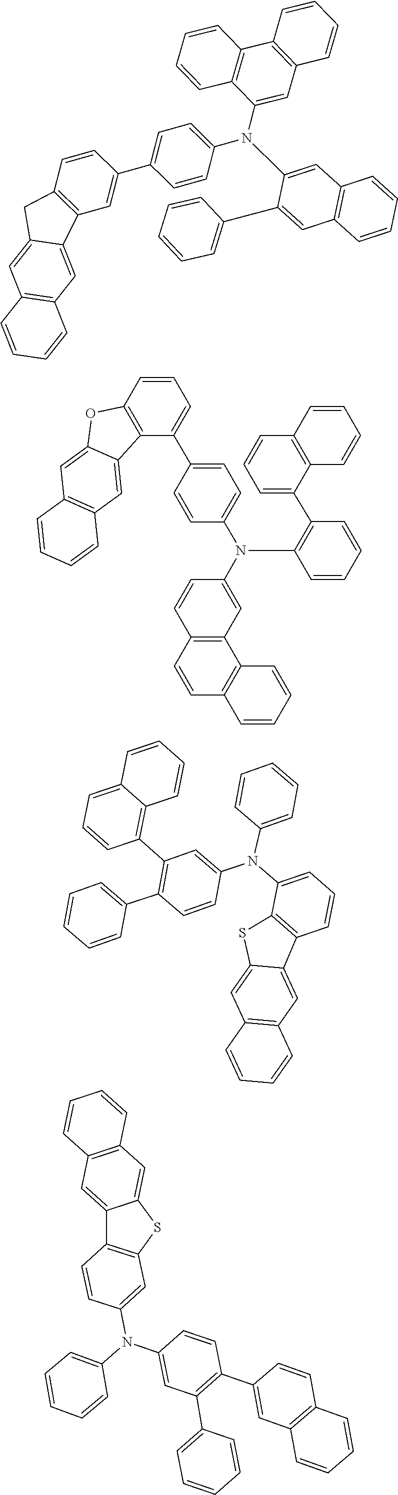 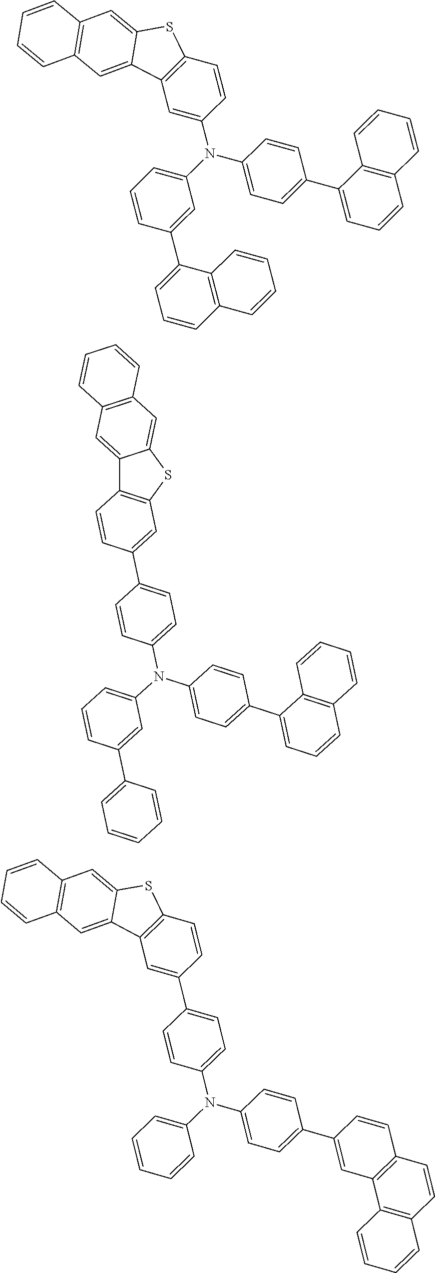 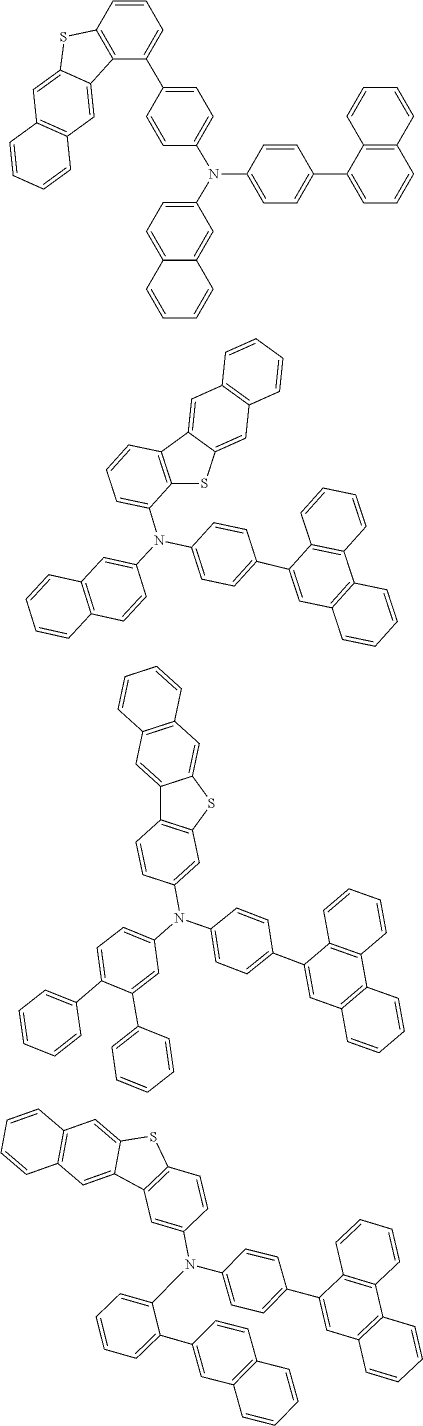 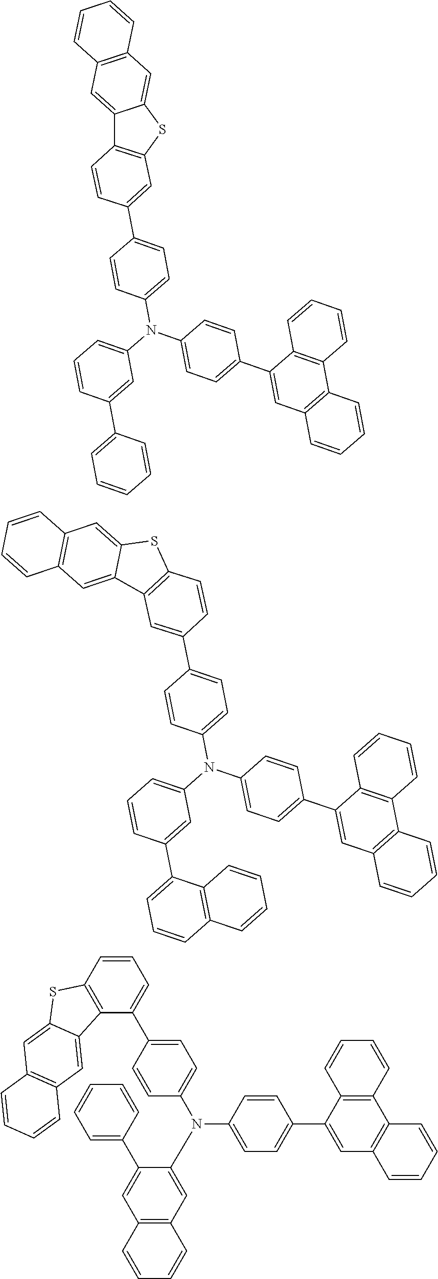 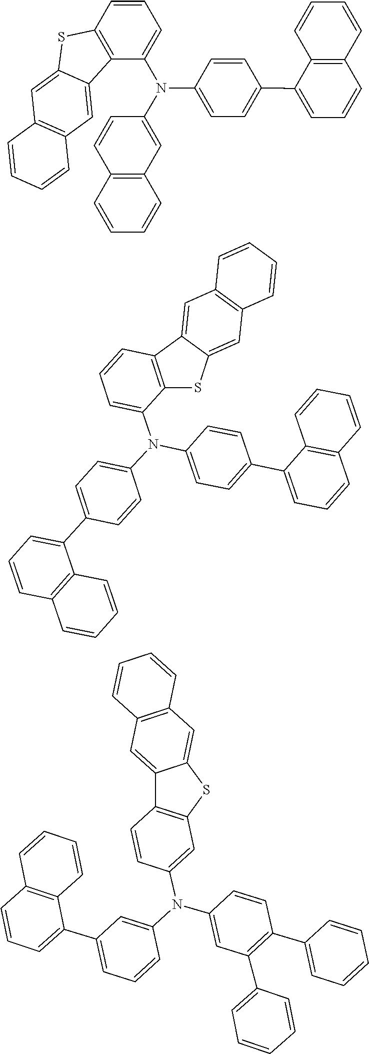 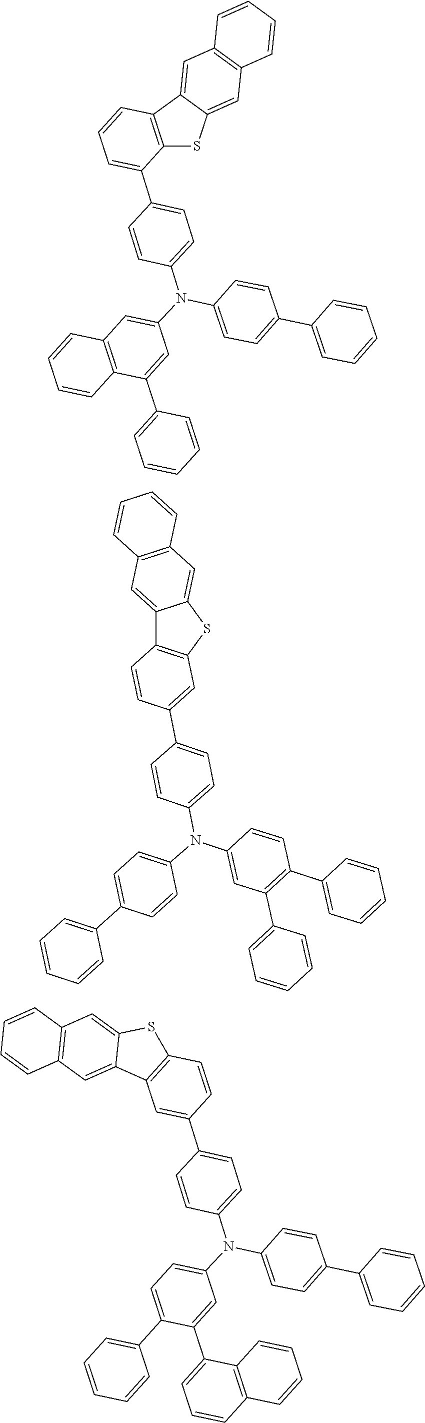 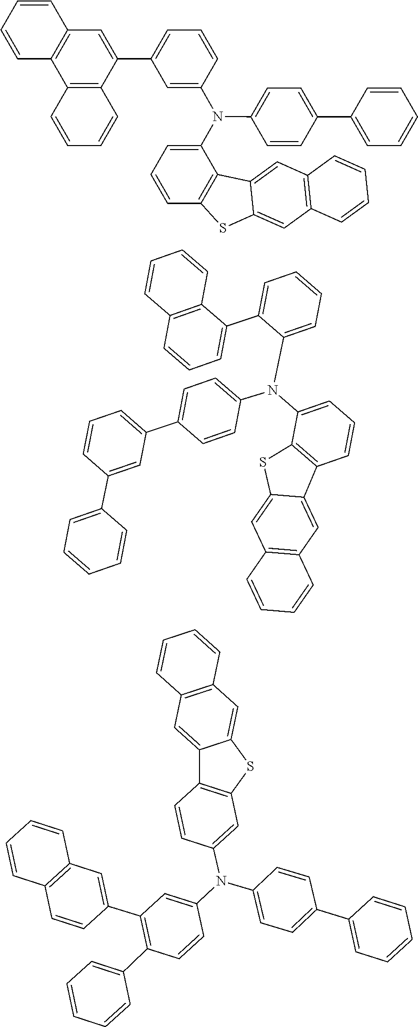 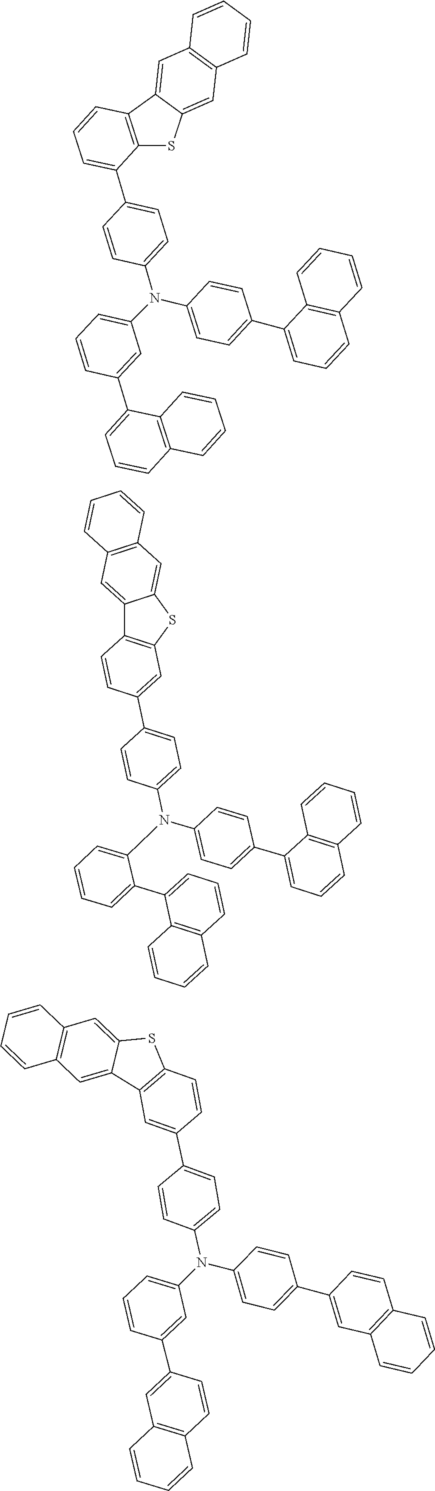 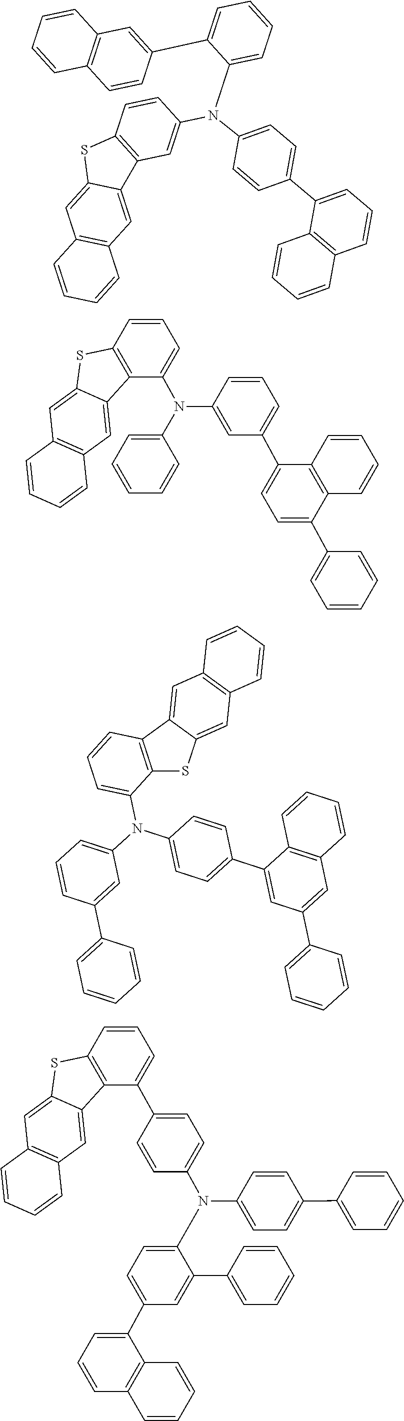 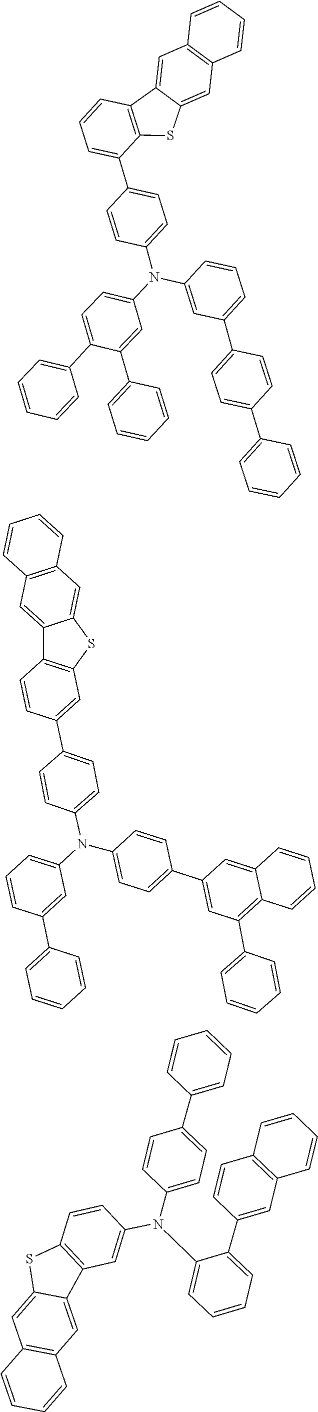 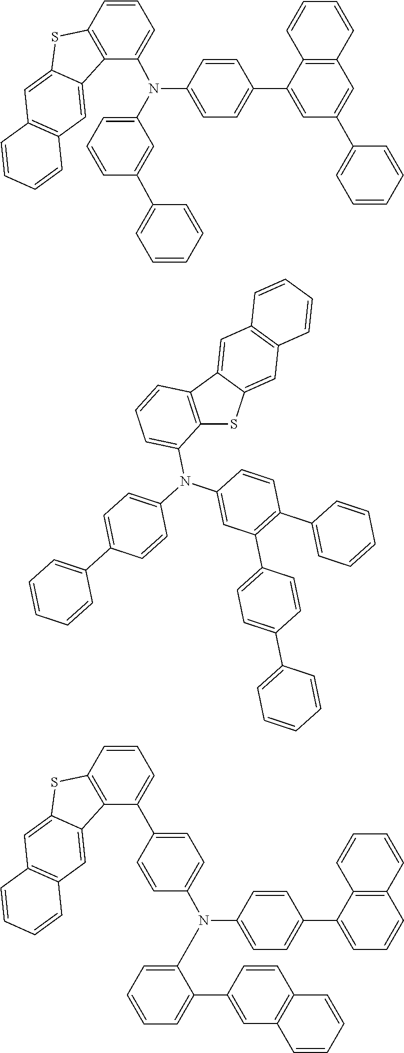 [SMILES: c1ccc(-c2cccc(N(c3ccc(-c4cc(-c5ccccc5)cc5ccccc45)cc3)c3cccc4sc5cc6ccccc6cc5c34)c2)cc1 ; c1ccc(-c2ccc(-c3cc(N(c4ccc(-c5ccccc5)cc4)c4cccc5c4sc4cc6ccccc6cc45)ccc3-c3ccccc3)cc2)cc1 ; c1ccc(N(c2ccc(-c3cccc4ccccc34)cc2)c2ccc(-c3cccc4sc5cc6ccccc6cc5c34)cc2)c(-c2ccc3ccccc3c2)c1] 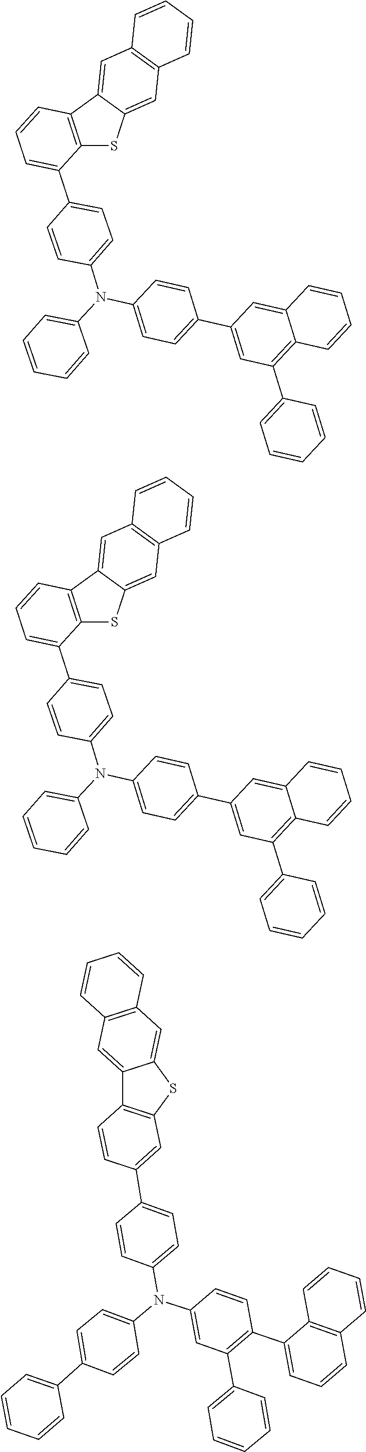 [SMILES: c1ccc(-c2cc(-c3ccc(N(c4ccccc4)c4ccc(-c5cccc6c5sc5cc7ccccc7cc56)cc4)cc3)cc3ccccc23)cc1 ; c1ccc(-c2cc(-c3ccc(N(c4ccccc4)c4ccc(-c5cccc6c5sc5cc7ccccc7cc56)cc4)cc3)cc3ccccc23)cc1 ; c1ccc(-c2ccc(N(c3ccc(-c4ccc5c(c4)sc4cc6ccccc6cc45)cc3)c3ccc(-c4cccc5ccccc45)c(-c4ccccc4)c3)cc2)cc1] 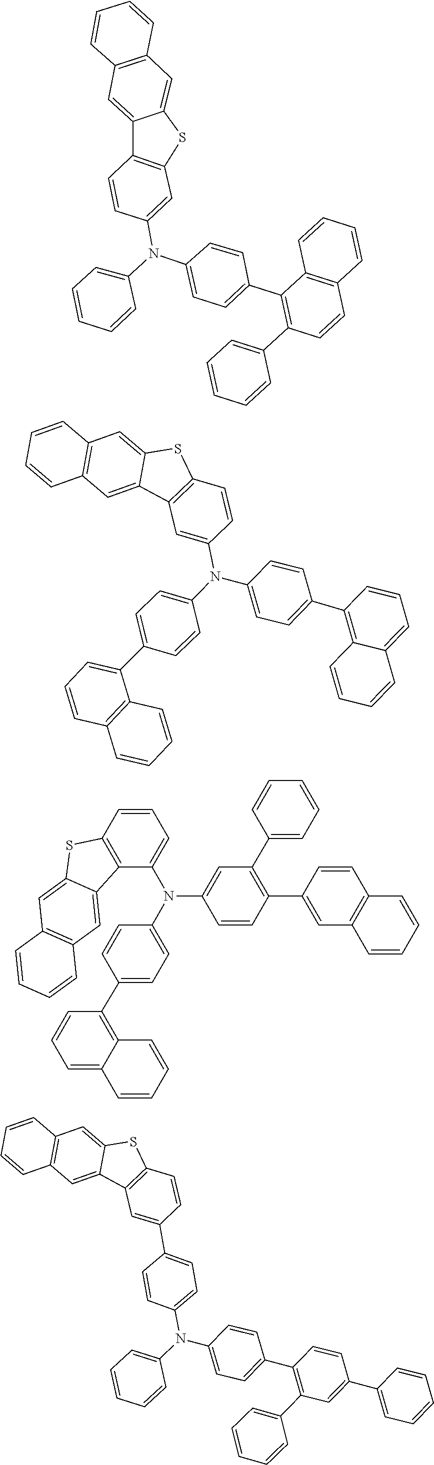 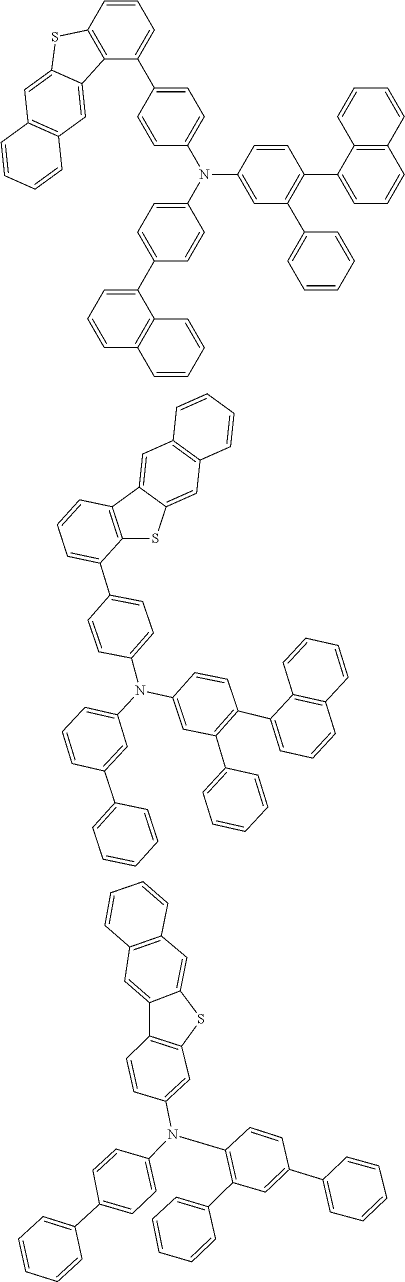 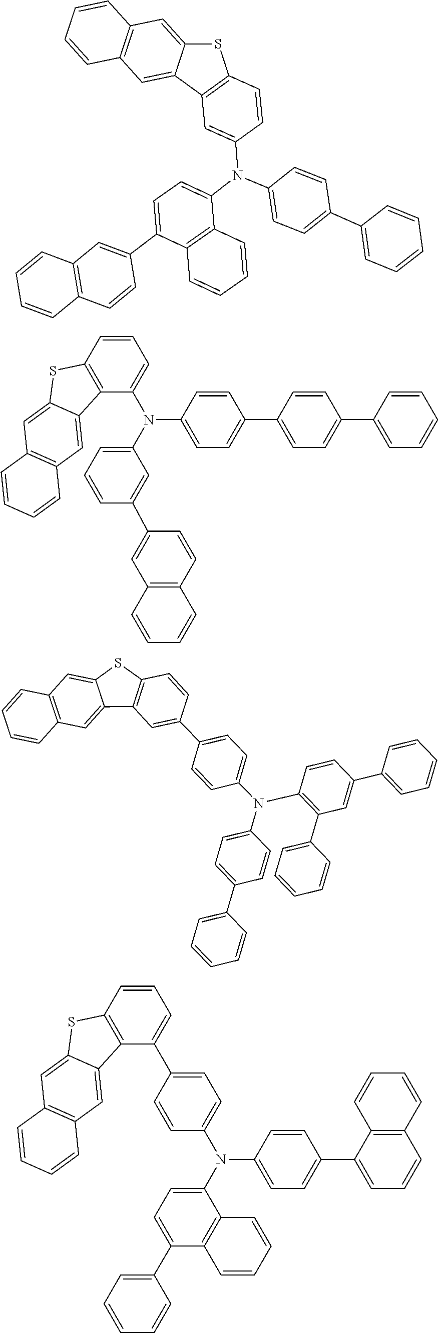 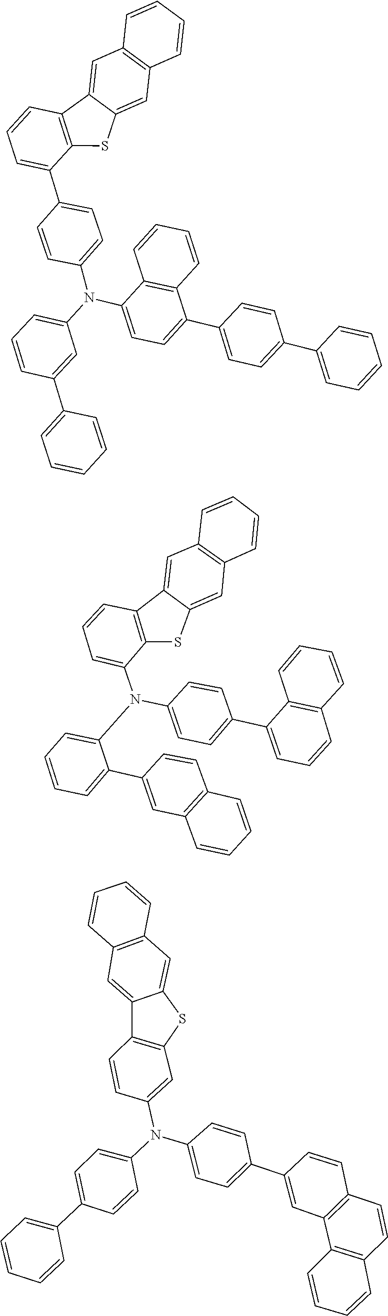 [SMILES: c1ccc(-c2ccc(-c3ccc(N(c4ccc(-c5cccc6c5sc5cc7ccccc7cc56)cc4)c4cccc(-c5ccccc5)c4)c4ccccc34)cc2)cc1 ; c1ccc(N(c2ccc(-c3cccc4ccccc34)cc2)c2cccc3c2sc2cc4ccccc4cc23)c(-c2ccc3ccccc3c2)c1 ; c1ccc(-c2ccc(N(c3ccc(-c4ccc5ccc6ccccc6c5c4)cc3)c3ccc4c(c3)sc3cc5ccccc5cc34)cc2)cc1] 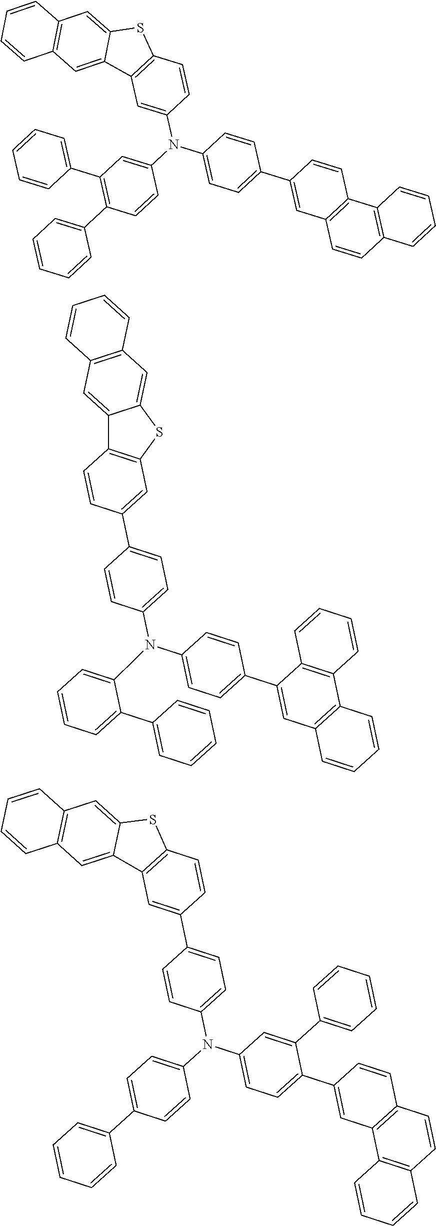 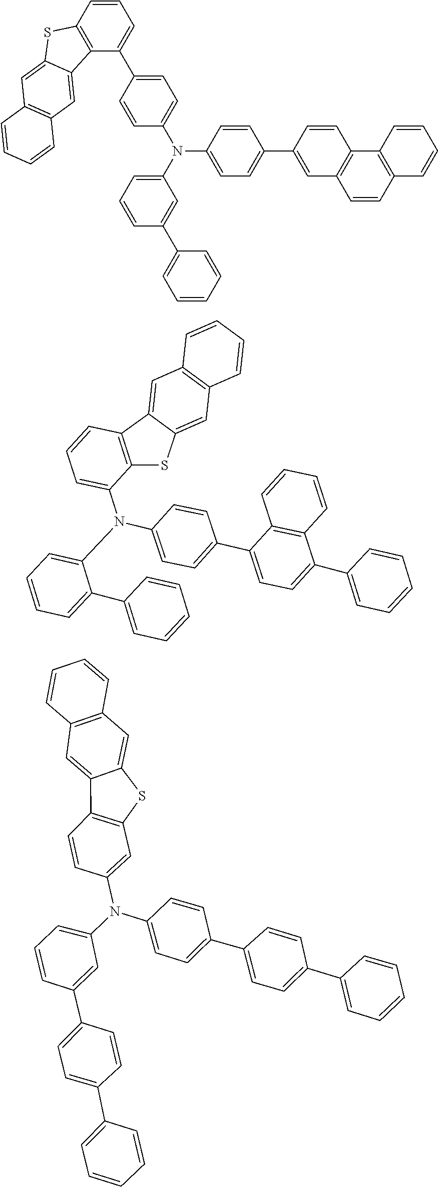 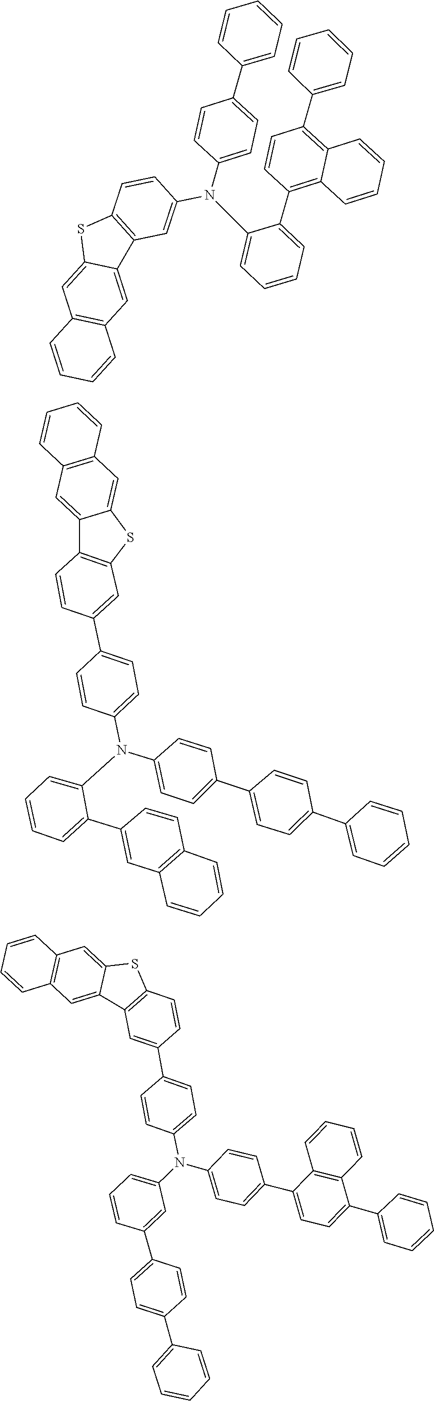 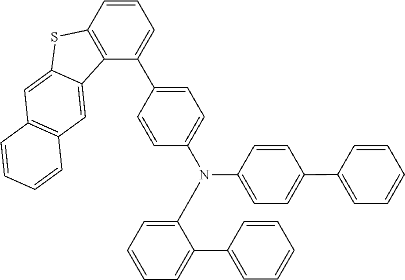 [SMILES: c1ccc(-c2ccc(N(c3ccc(-c4cccc5sc6cc7ccccc7cc6c45)cc3)c3ccccc3-c3ccccc3)cc2)cc1] 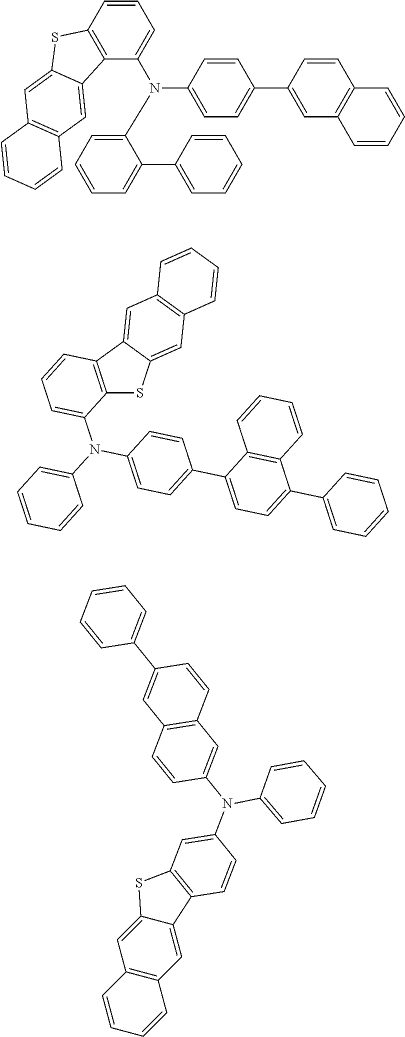 [SMILES: c1ccc(-c2ccccc2N(c2ccc(-c3ccc4ccccc4c3)cc2)c2cccc3sc4cc5ccccc5cc4c23)cc1 ; c1ccc(-c2ccc(-c3ccc(N(c4ccccc4)c4cccc5c4sc4cc6ccccc6cc45)cc3)c3ccccc23)cc1 ; c1ccc(-c2ccc3cc(N(c4ccccc4)c4ccc5c(c4)sc4cc6ccccc6cc45)ccc3c2)cc1] 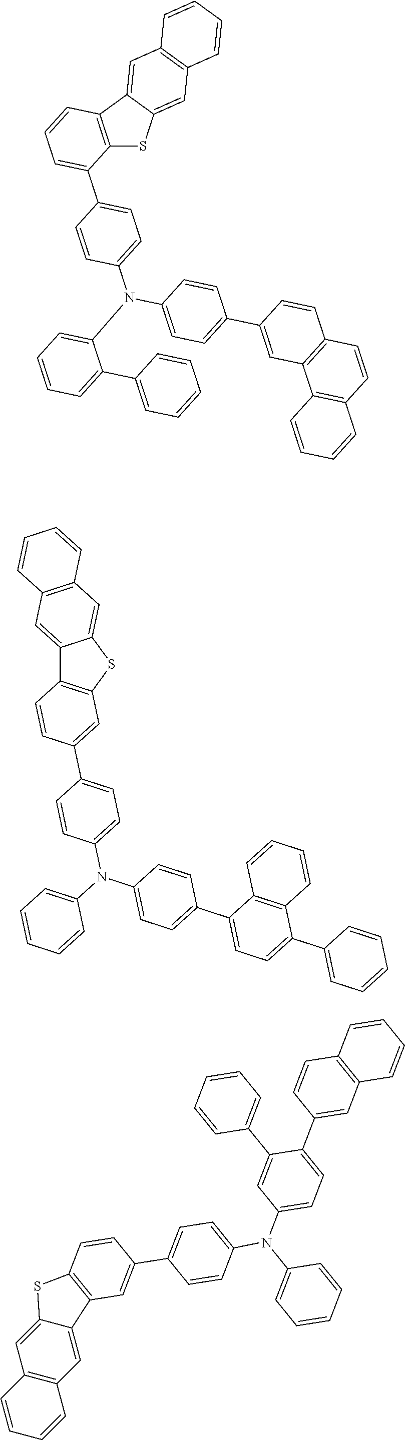 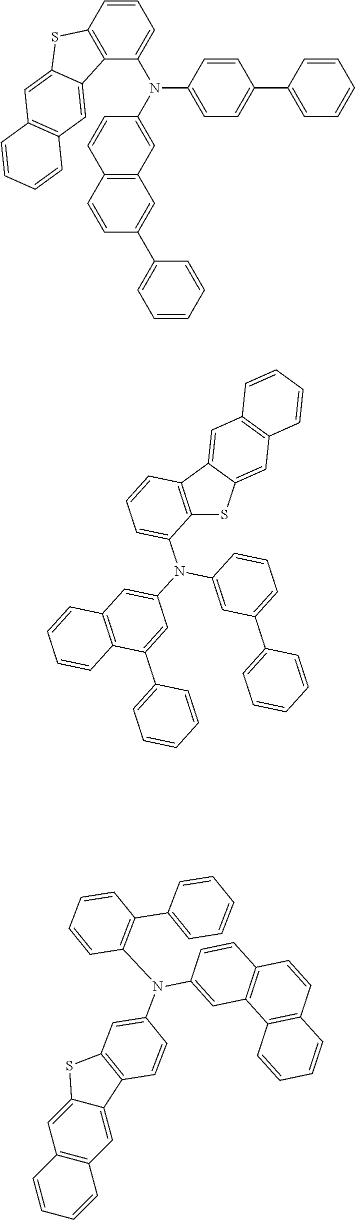 [SMILES: c1ccc(-c2ccc(N(c3ccc4ccc(-c5ccccc5)cc4c3)c3cccc4sc5cc6ccccc6cc5c34)cc2)cc1 ; c1ccc(-c2cccc(N(c3cc(-c4ccccc4)c4ccccc4c3)c3cccc4c3sc3cc5ccccc5cc34)c2)cc1 ; c1ccc(-c2ccccc2N(c2ccc3c(c2)sc2cc4ccccc4cc23)c2ccc3ccc4ccccc4c3c2)cc1] 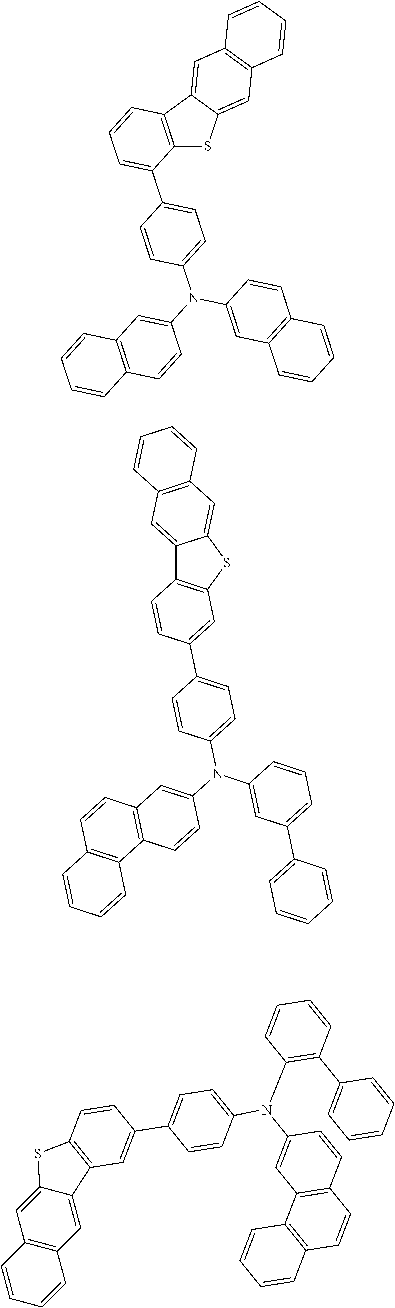 [SMILES: c1ccc2cc(N(c3ccc(-c4cccc5c4sc4cc6ccccc6cc45)cc3)c3ccc4ccccc4c3)ccc2c1 ; c1ccc(-c2cccc(N(c3ccc(-c4ccc5c(c4)sc4cc6ccccc6cc45)cc3)c3ccc4c(ccc5ccccc54)c3)c2)cc1 ; c1ccc(-c2ccccc2N(c2ccc(-c3ccc4sc5cc6ccccc6cc5c4c3)cc2)c2ccc3ccc4ccccc4c3c2)cc1] 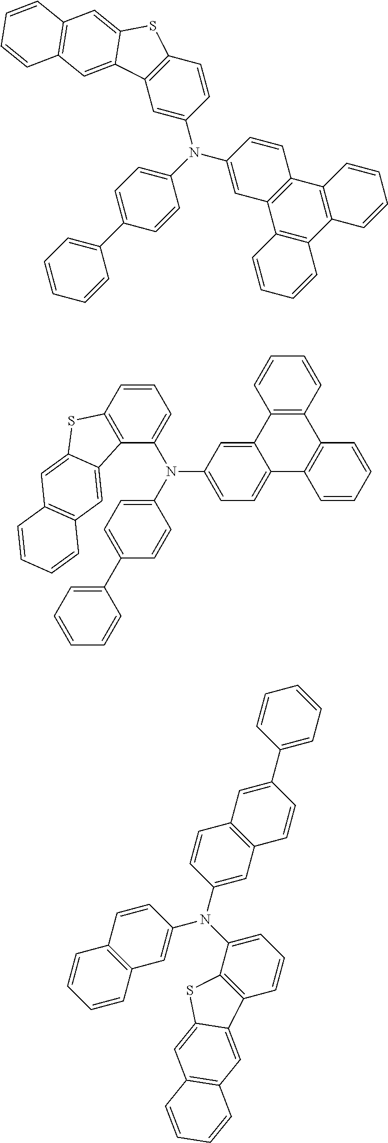 [SMILES: c1ccc(-c2ccc(N(c3ccc4sc5cc6ccccc6cc5c4c3)c3ccc4c5ccccc5c5ccccc5c4c3)cc2)cc1 ; c1ccc(-c2ccc(N(c3ccc4c5ccccc5c5ccccc5c4c3)c3cccc4sc5cc6ccccc6cc5c34)cc2)cc1 ; c1ccc(-c2ccc3cc(N(c4ccc5ccccc5c4)c4cccc5c4sc4cc6ccccc6cc45)ccc3c2)cc1] 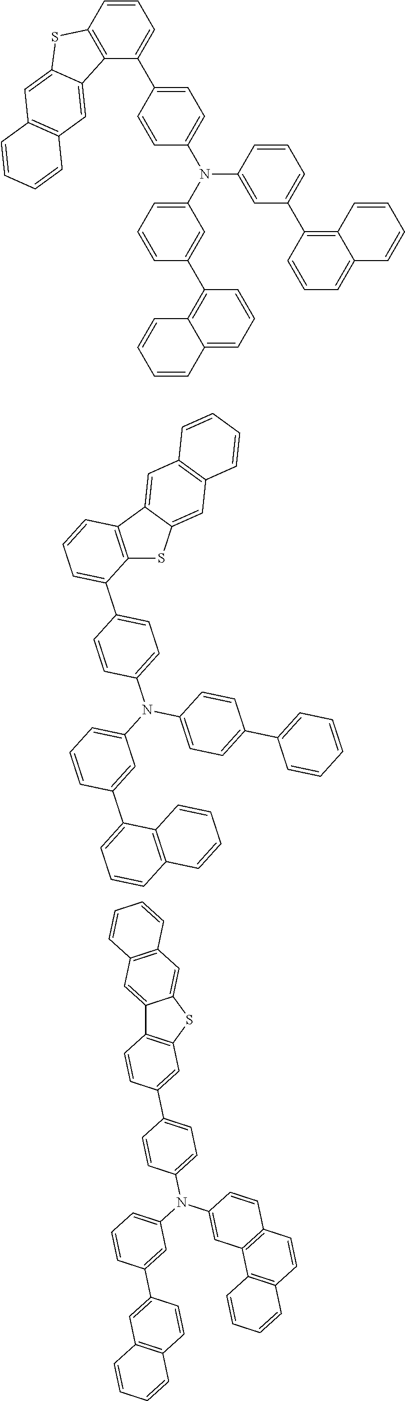 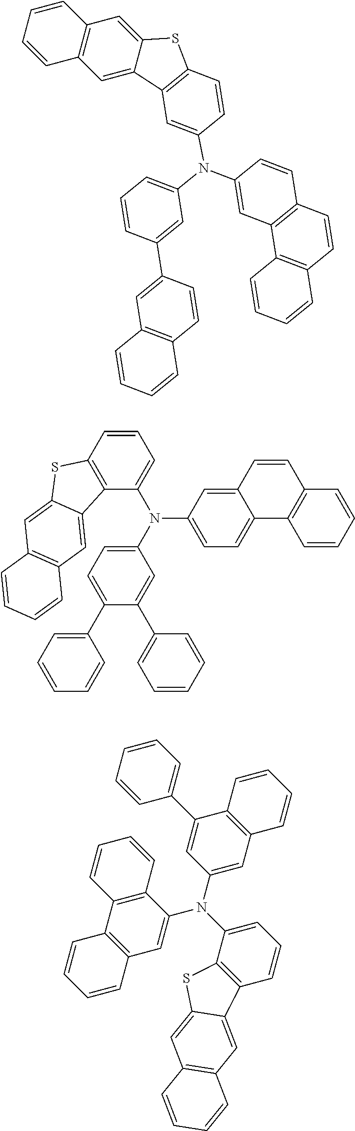 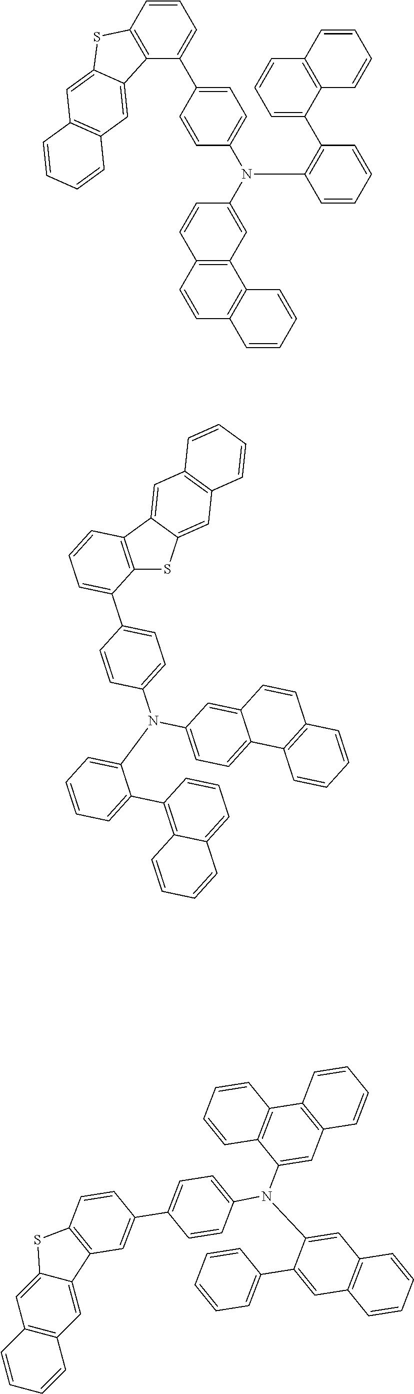 [SMILES: c1ccc(N(c2ccc(-c3cccc4sc5cc6ccccc6cc5c34)cc2)c2ccc3ccc4ccccc4c3c2)c(-c2cccc3ccccc23)c1 ; c1ccc(N(c2ccc(-c3cccc4c3sc3cc5ccccc5cc34)cc2)c2ccc3c(ccc4ccccc43)c2)c(-c2cccc3ccccc23)c1 ; c1ccc(-c2cc3ccccc3cc2N(c2ccc(-c3ccc4sc5cc6ccccc6cc5c4c3)cc2)c2cc3ccccc3c3ccccc23)cc1] 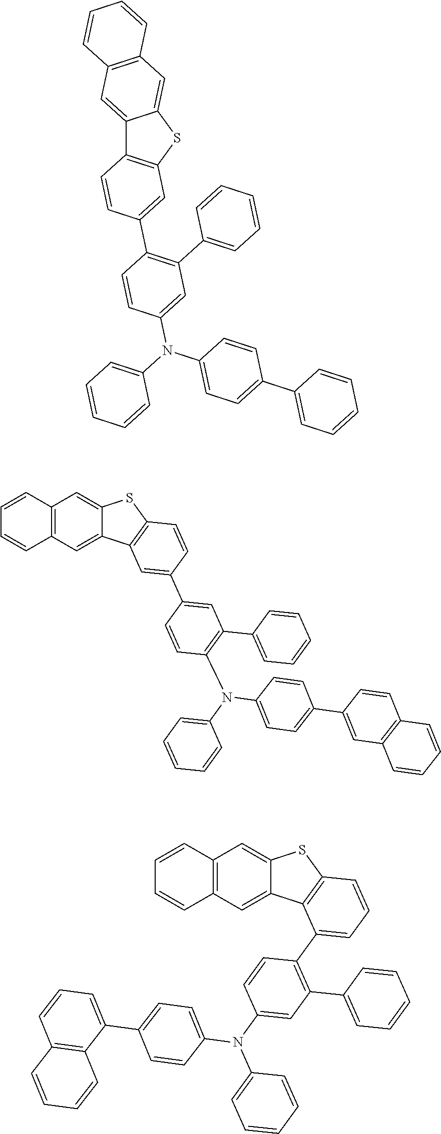 [SMILES: c1ccc(-c2ccc(N(c3ccccc3)c3ccc(-c4ccc5c(c4)sc4cc6ccccc6cc45)c(-c4ccccc4)c3)cc2)cc1 ; c1ccc(-c2cc(-c3ccc4sc5cc6ccccc6cc5c4c3)ccc2N(c2ccccc2)c2ccc(-c3ccc4ccccc4c3)cc2)cc1 ; c1ccc(-c2cc(N(c3ccccc3)c3ccc(-c4cccc5ccccc45)cc3)ccc2-c2cccc3sc4cc5ccccc5cc4c23)cc1] 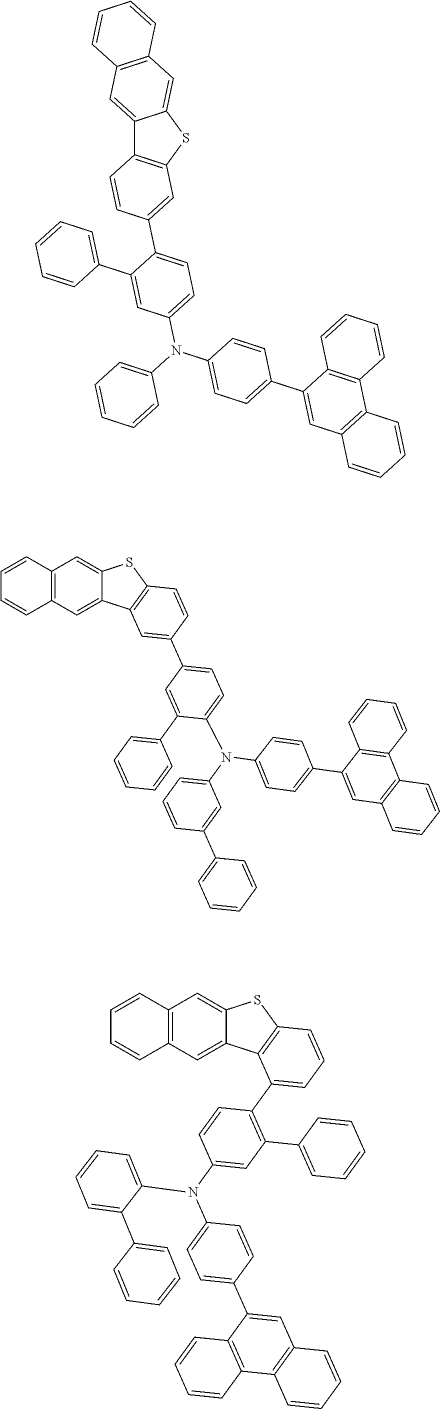 [SMILES: c1ccc(-c2cc(N(c3ccccc3)c3ccc(-c4cc5ccccc5c5ccccc45)cc3)ccc2-c2ccc3c(c2)sc2cc4ccccc4cc23)cc1 ; c1ccc(-c2cccc(N(c3ccc(-c4cc5ccccc5c5ccccc45)cc3)c3ccc(-c4ccc5sc6cc7ccccc7cc6c5c4)cc3-c3ccccc3)c2)cc1 ; c1ccc(-c2cc(N(c3ccc(-c4cc5ccccc5c5ccccc45)cc3)c3ccccc3-c3ccccc3)ccc2-c2cccc3sc4cc5ccccc5cc4c23)cc1] 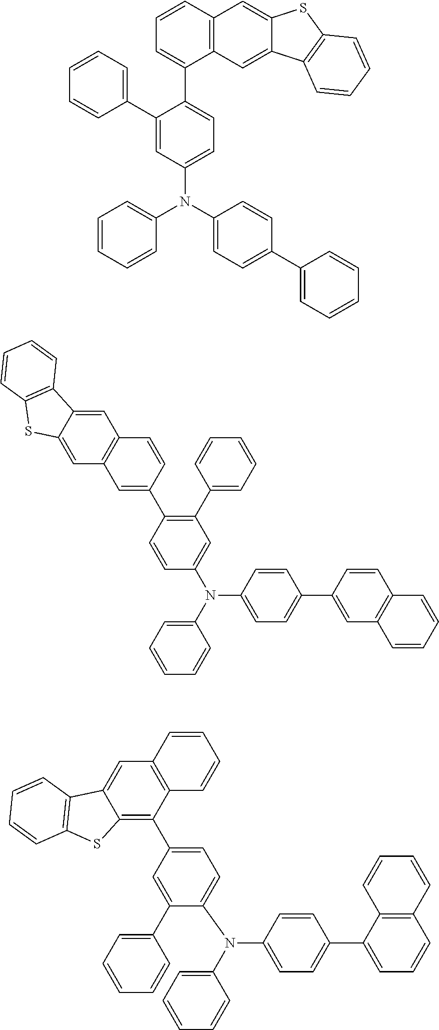 [SMILES: c1ccc(-c2ccc(N(c3ccccc3)c3ccc(-c4cccc5cc6sc7ccccc7c6cc45)c(-c4ccccc4)c3)cc2)cc1 ; c1ccc(-c2cc(N(c3ccccc3)c3ccc(-c4ccc5ccccc5c4)cc3)ccc2-c2ccc3cc4c(cc3c2)sc2ccccc24)cc1 ; c1ccc(-c2cc(-c3c4ccccc4cc4c3sc3ccccc34)ccc2N(c2ccccc2)c2ccc(-c3cccc4ccccc34)cc2)cc1] 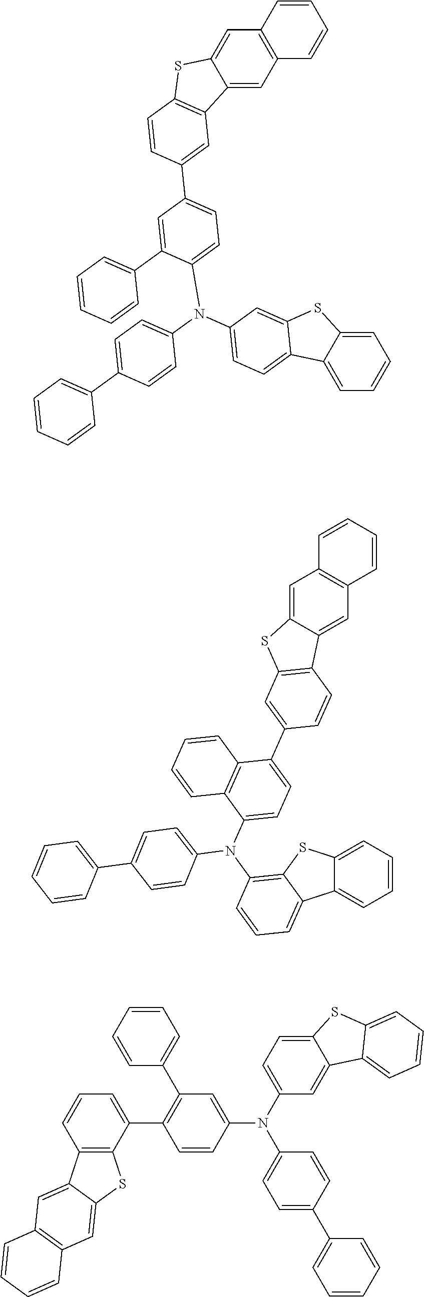 [SMILES: c1ccc(-c2ccc(N(c3ccc4c(c3)sc3ccccc34)c3ccc(-c4ccc5sc6cc7ccccc7cc6c5c4)cc3-c3ccccc3)cc2)cc1 ; c1ccc(-c2ccc(N(c3ccc(-c4ccc5c(c4)sc4cc6ccccc6cc45)c4ccccc34)c3cccc4c3sc3ccccc34)cc2)cc1 ; c1ccc(-c2ccc(N(c3ccc(-c4cccc5c4sc4cc6ccccc6cc45)c(-c4ccccc4)c3)c3ccc4sc5ccccc5c4c3)cc2)cc1] 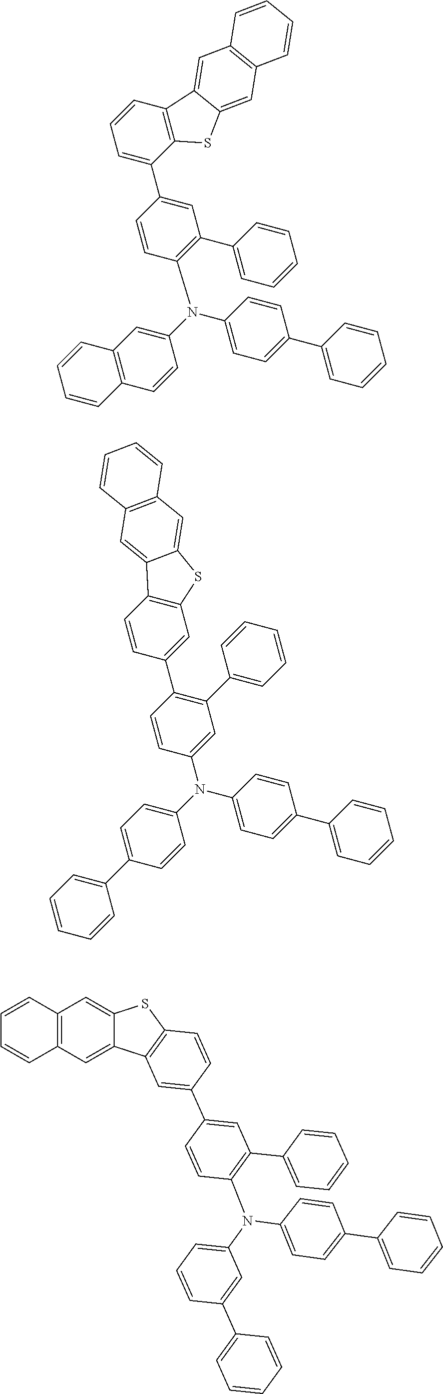 [SMILES: c1ccc(-c2ccc(N(c3ccc4ccccc4c3)c3ccc(-c4cccc5c4sc4cc6ccccc6cc45)cc3-c3ccccc3)cc2)cc1 ; c1ccc(-c2ccc(N(c3ccc(-c4ccccc4)cc3)c3ccc(-c4ccc5c(c4)sc4cc6ccccc6cc45)c(-c4ccccc4)c3)cc2)cc1 ; c1ccc(-c2ccc(N(c3cccc(-c4ccccc4)c3)c3ccc(-c4ccc5sc6cc7ccccc7cc6c5c4)cc3-c3ccccc3)cc2)cc1] 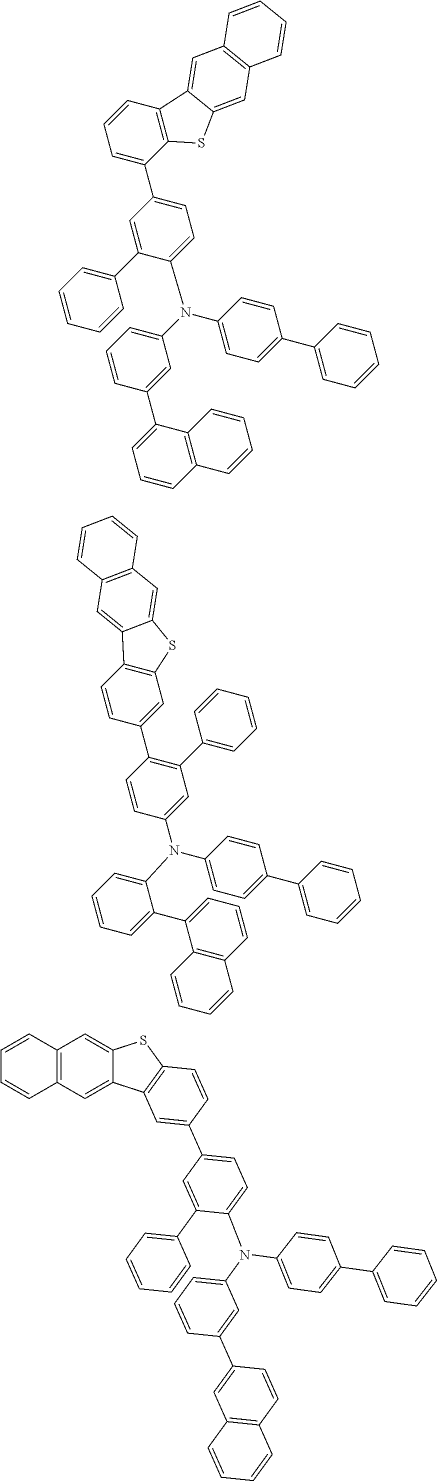 [SMILES: c1ccc(-c2ccc(N(c3cccc(-c4cccc5ccccc45)c3)c3ccc(-c4cccc5c4sc4cc6ccccc6cc45)cc3-c3ccccc3)cc2)cc1 ; c1ccc(-c2ccc(N(c3ccc(-c4ccc5c(c4)sc4cc6ccccc6cc45)c(-c4ccccc4)c3)c3ccccc3-c3cccc4ccccc34)cc2)cc1 ; c1ccc(-c2ccc(N(c3cccc(-c4ccc5ccccc5c4)c3)c3ccc(-c4ccc5sc6cc7ccccc7cc6c5c4)cc3-c3ccccc3)cc2)cc1] 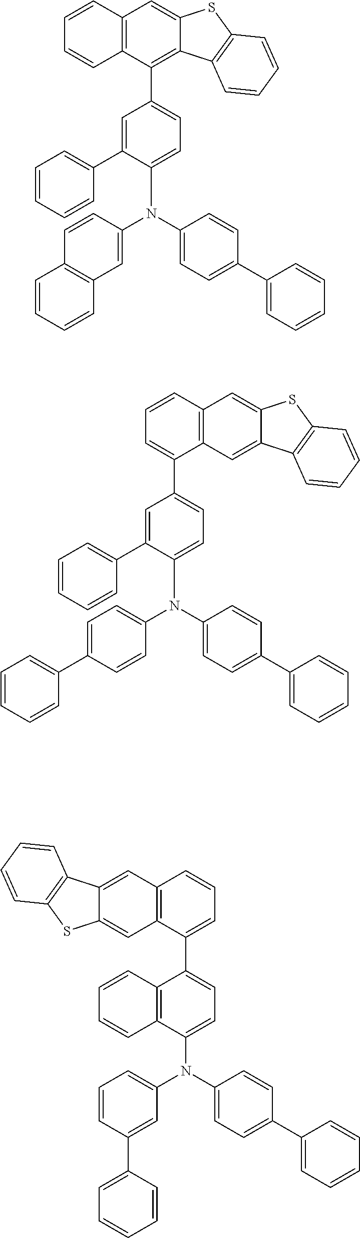 [SMILES: c1ccc(-c2ccc(N(c3ccc4ccccc4c3)c3ccc(-c4c5ccccc5cc5sc6ccccc6c45)cc3-c3ccccc3)cc2)cc1 ; c1ccc(-c2ccc(N(c3ccc(-c4ccccc4)cc3)c3ccc(-c4cccc5cc6sc7ccccc7c6cc45)cc3-c3ccccc3)cc2)cc1 ; c1ccc(-c2ccc(N(c3cccc(-c4ccccc4)c3)c3ccc(-c4cccc5cc6c(cc45)sc4ccccc46)c4ccccc34)cc2)cc1] 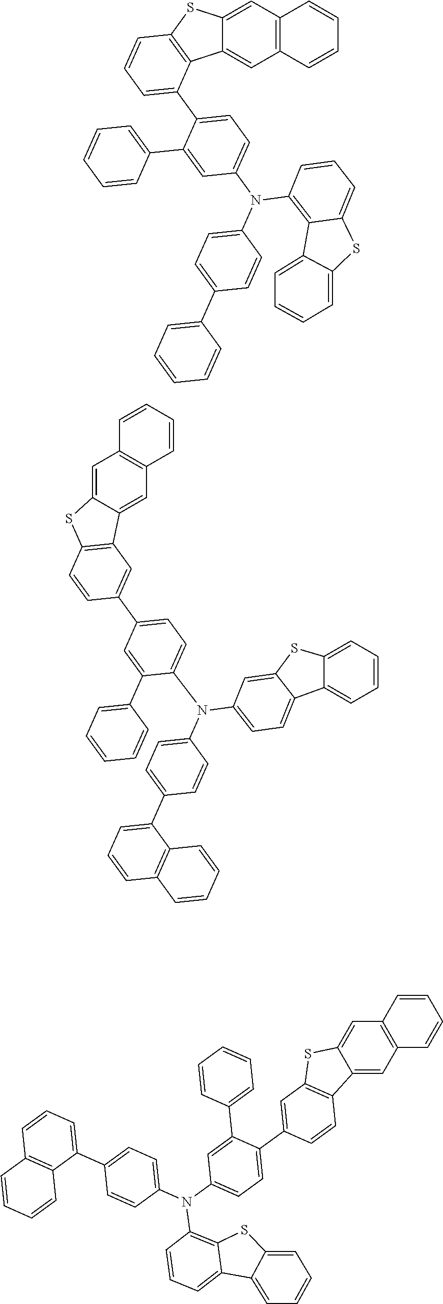 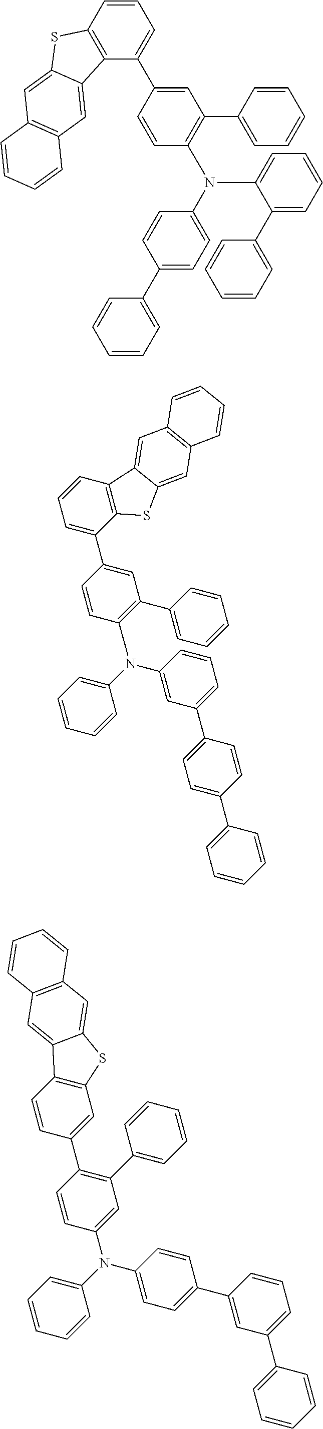 [SMILES: c1ccc(-c2ccc(N(c3ccccc3-c3ccccc3)c3ccc(-c4cccc5sc6cc7ccccc7cc6c45)cc3-c3ccccc3)cc2)cc1 ; c1ccc(-c2ccc(-c3cccc(N(c4ccccc4)c4ccc(-c5cccc6c5sc5cc7ccccc7cc56)cc4-c4ccccc4)c3)cc2)cc1 ; c1ccc(-c2cccc(-c3ccc(N(c4ccccc4)c4ccc(-c5ccc6c(c5)sc5cc7ccccc7cc56)c(-c5ccccc5)c4)cc3)c2)cc1] 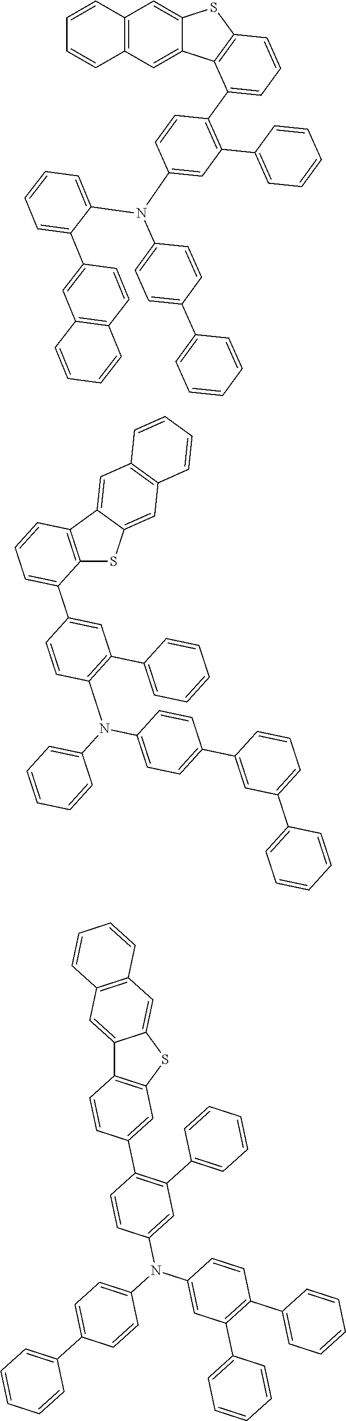 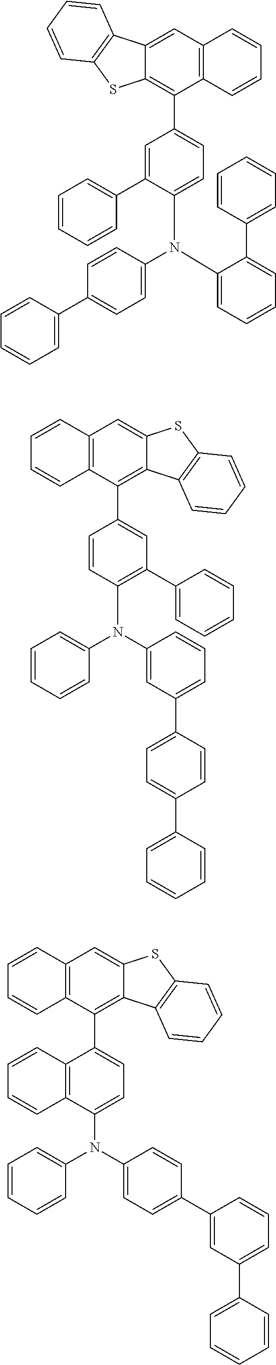 [SMILES: c1ccc(-c2ccc(N(c3ccccc3-c3ccccc3)c3ccc(-c4c5ccccc5cc5c4sc4ccccc45)cc3-c3ccccc3)cc2)cc1 ; c1ccc(-c2ccc(-c3cccc(N(c4ccccc4)c4ccc(-c5c6ccccc6cc6sc7ccccc7c56)cc4-c4ccccc4)c3)cc2)cc1 ; c1ccc(-c2cccc(-c3ccc(N(c4ccccc4)c4ccc(-c5c6ccccc6cc6sc7ccccc7c56)c5ccccc45)cc3)c2)cc1] 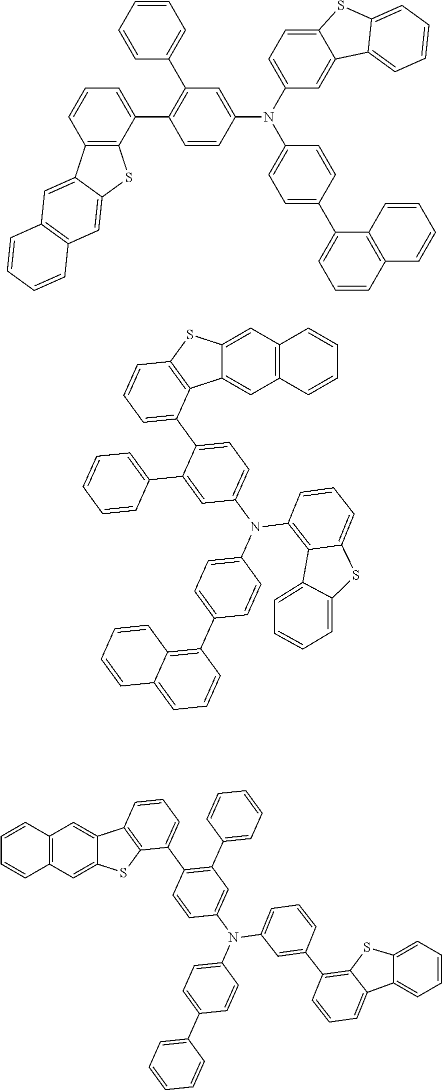 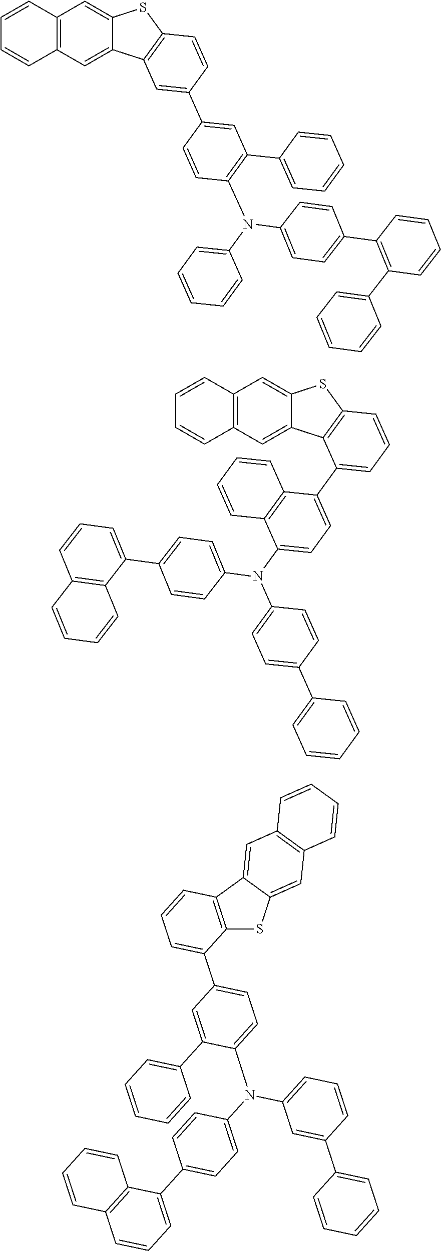 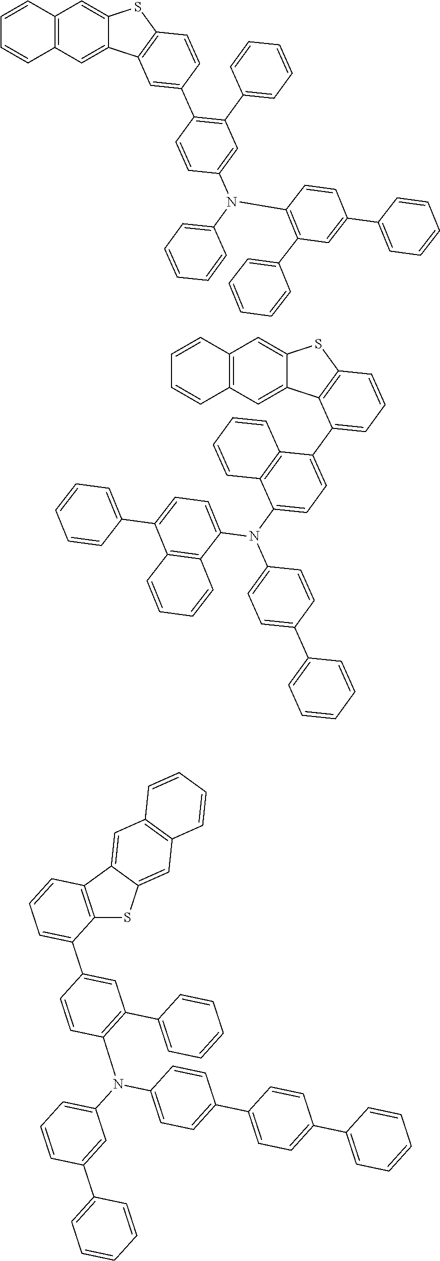 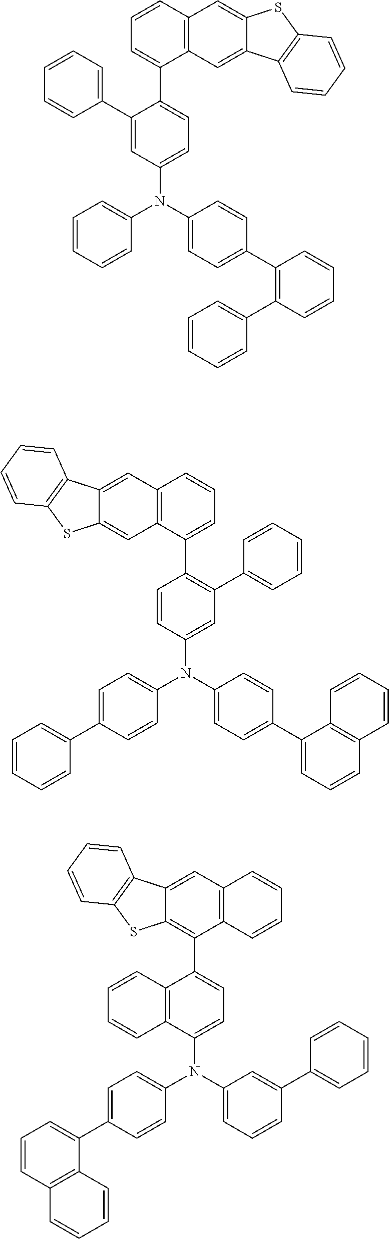 [SMILES: c1ccc(-c2ccccc2-c2ccc(N(c3ccccc3)c3ccc(-c4cccc5cc6sc7ccccc7c6cc45)c(-c4ccccc4)c3)cc2)cc1 ; c1ccc(-c2ccc(N(c3ccc(-c4cccc5ccccc45)cc3)c3ccc(-c4cccc5cc6c(cc45)sc4ccccc46)c(-c4ccccc4)c3)cc2)cc1 ; c1ccc(-c2cccc(N(c3ccc(-c4cccc5ccccc45)cc3)c3ccc(-c4c5ccccc5cc5c4sc4ccccc45)c4ccccc34)c2)cc1] 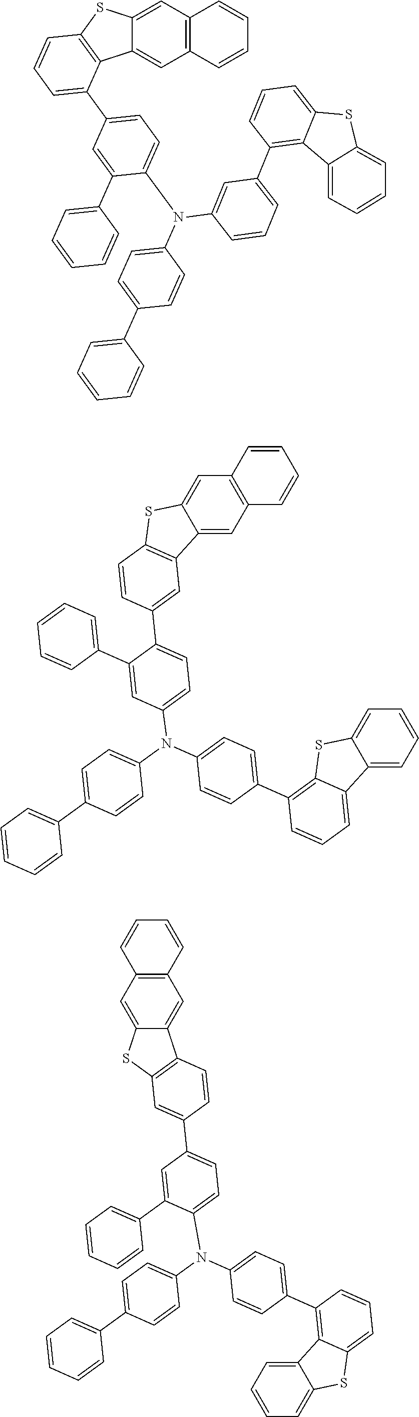 [SMILES: c1ccc(-c2ccc(N(c3cccc(-c4cccc5sc6ccccc6c45)c3)c3ccc(-c4cccc5sc6cc7ccccc7cc6c45)cc3-c3ccccc3)cc2)cc1 ; c1ccc(-c2ccc(N(c3ccc(-c4cccc5c4sc4ccccc45)cc3)c3ccc(-c4ccc5sc6cc7ccccc7cc6c5c4)c(-c4ccccc4)c3)cc2)cc1 ; c1ccc(-c2ccc(N(c3ccc(-c4cccc5sc6ccccc6c45)cc3)c3ccc(-c4ccc5c(c4)sc4cc6ccccc6cc45)cc3-c3ccccc3)cc2)cc1] 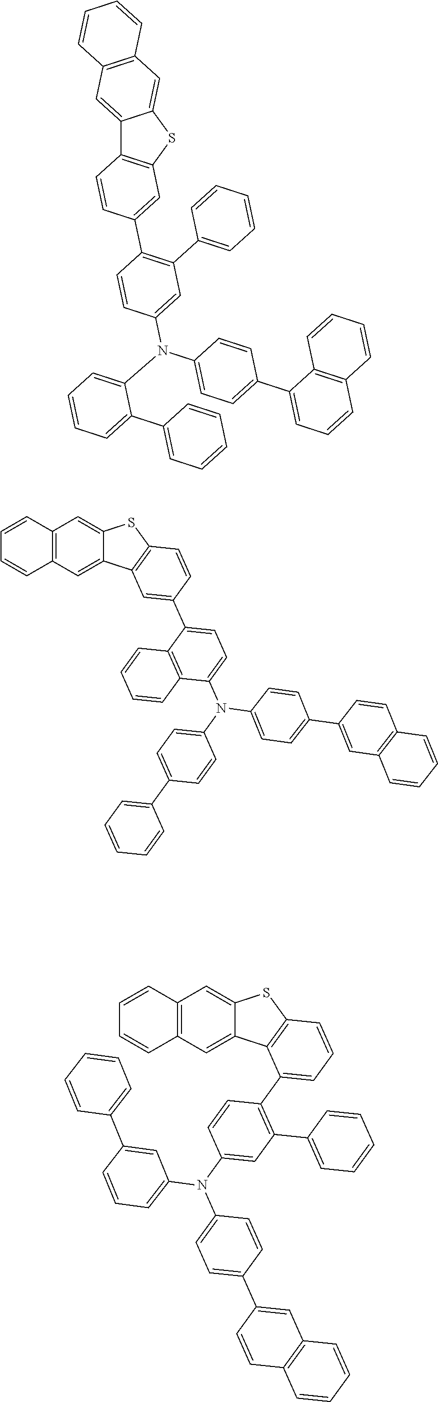 [SMILES: c1ccc(-c2cc(N(c3ccc(-c4cccc5ccccc45)cc3)c3ccccc3-c3ccccc3)ccc2-c2ccc3c(c2)sc2cc4ccccc4cc23)cc1 ; c1ccc(-c2ccc(N(c3ccc(-c4ccc5ccccc5c4)cc3)c3ccc(-c4ccc5sc6cc7ccccc7cc6c5c4)c4ccccc34)cc2)cc1 ; c1ccc(-c2cccc(N(c3ccc(-c4ccc5ccccc5c4)cc3)c3ccc(-c4cccc5sc6cc7ccccc7cc6c45)c(-c4ccccc4)c3)c2)cc1] 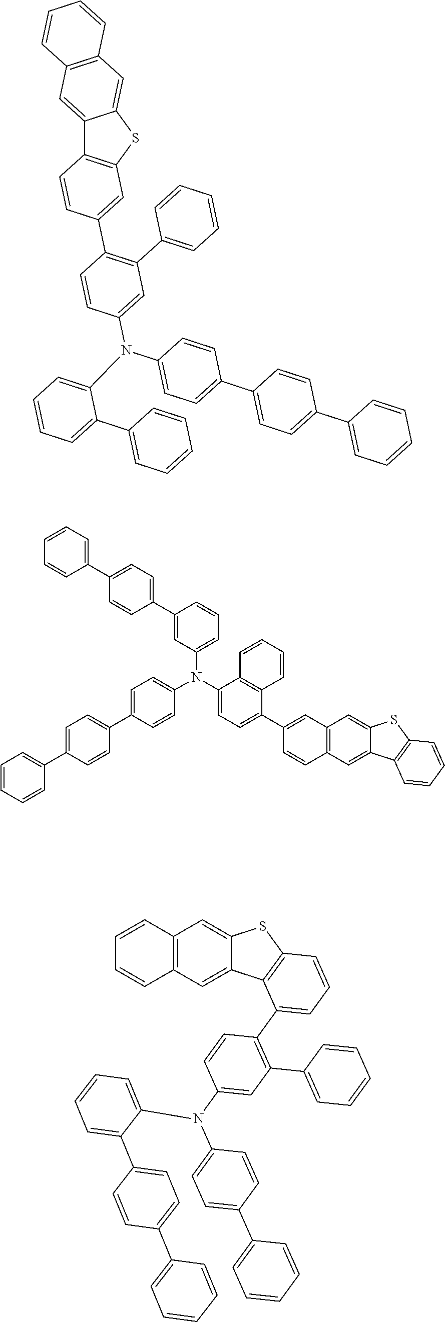 [SMILES: c1ccc(-c2ccc(-c3ccc(N(c4ccc(-c5ccc6c(c5)sc5cc7ccccc7cc56)c(-c5ccccc5)c4)c4ccccc4-c4ccccc4)cc3)cc2)cc1 ; c1ccc(-c2ccc(-c3ccc(N(c4cccc(-c5ccc(-c6ccccc6)cc5)c4)c4ccc(-c5ccc6cc7c(cc6c5)sc5ccccc57)c5ccccc45)cc3)cc2)cc1 ; c1ccc(-c2ccc(-c3ccccc3N(c3ccc(-c4ccccc4)cc3)c3ccc(-c4cccc5sc6cc7ccccc7cc6c45)c(-c4ccccc4)c3)cc2)cc1] 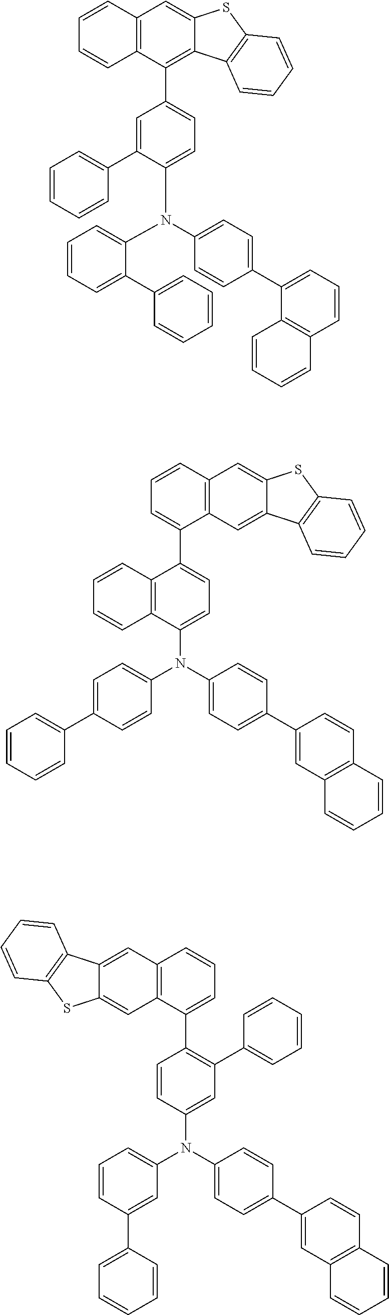 [SMILES: c1ccc(-c2ccccc2N(c2ccc(-c3cccc4ccccc34)cc2)c2ccc(-c3c4ccccc4cc4sc5ccccc5c34)cc2-c2ccccc2)cc1 ; c1ccc(-c2ccc(N(c3ccc(-c4ccc5ccccc5c4)cc3)c3ccc(-c4cccc5cc6sc7ccccc7c6cc45)c4ccccc34)cc2)cc1 ; c1ccc(-c2cccc(N(c3ccc(-c4ccc5ccccc5c4)cc3)c3ccc(-c4cccc5cc6c(cc45)sc4ccccc46)c(-c4ccccc4)c3)c2)cc1] 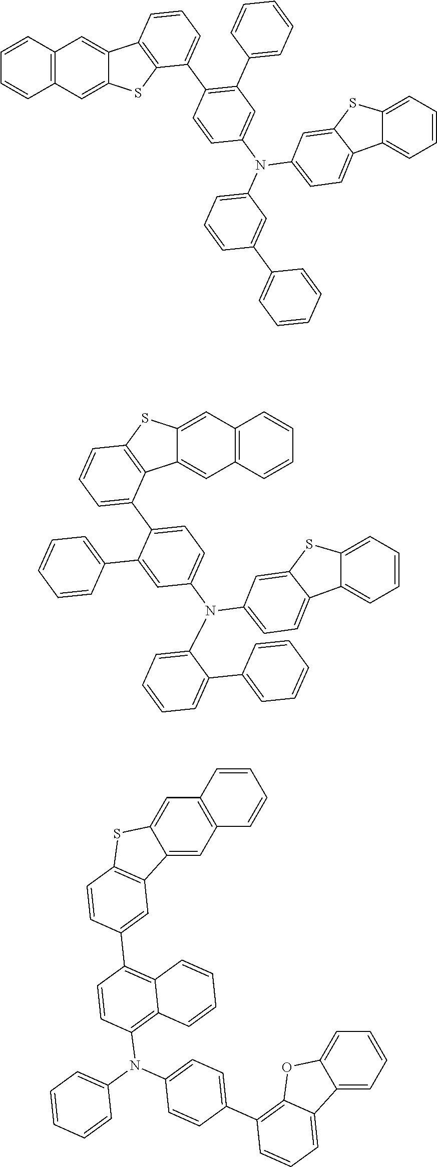 [SMILES: c1ccc(-c2cccc(N(c3ccc(-c4cccc5c4sc4cc6ccccc6cc45)c(-c4ccccc4)c3)c3ccc4c(c3)sc3ccccc34)c2)cc1 ; c1ccc(-c2cc(N(c3ccc4c(c3)sc3ccccc34)c3ccccc3-c3ccccc3)ccc2-c2cccc3sc4cc5ccccc5cc4c23)cc1 ; c1ccc(N(c2ccc(-c3cccc4c3oc3ccccc34)cc2)c2ccc(-c3ccc4sc5cc6ccccc6cc5c4c3)c3ccccc23)cc1] 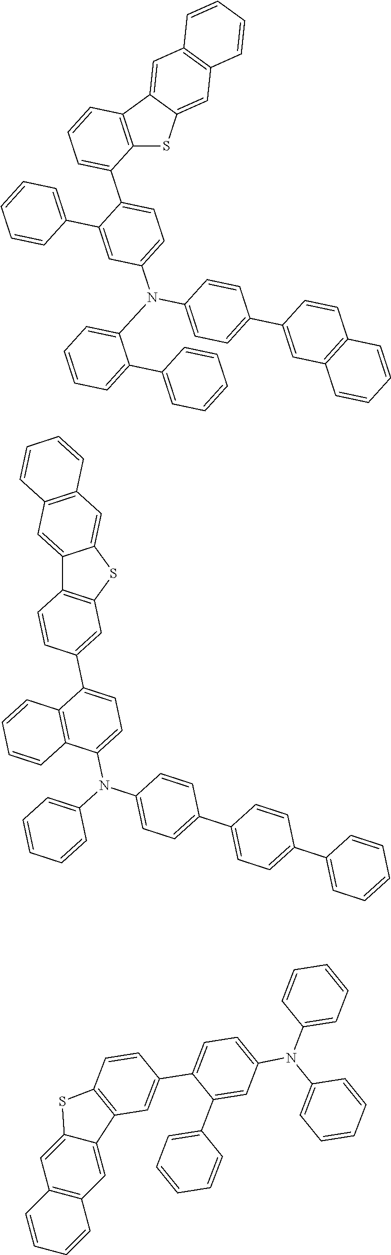 [SMILES: c1ccc(-c2cc(N(c3ccc(-c4ccc5ccccc5c4)cc3)c3ccccc3-c3ccccc3)ccc2-c2cccc3c2sc2cc4ccccc4cc23)cc1 ; c1ccc(-c2ccc(-c3ccc(N(c4ccccc4)c4ccc(-c5ccc6c(c5)sc5cc7ccccc7cc56)c5ccccc45)cc3)cc2)cc1 ; c1ccc(-c2cc(N(c3ccccc3)c3ccccc3)ccc2-c2ccc3sc4cc5ccccc5cc4c3c2)cc1] 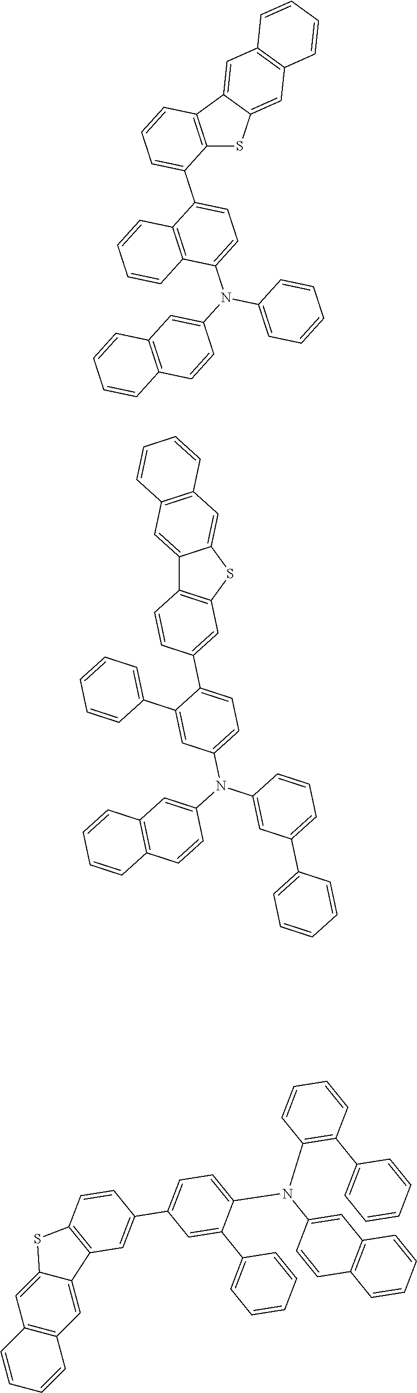 [SMILES: c1ccc(N(c2ccc3ccccc3c2)c2ccc(-c3cccc4c3sc3cc5ccccc5cc34)c3ccccc23)cc1 ; c1ccc(-c2cccc(N(c3ccc(-c4ccc5c(c4)sc4cc6ccccc6cc45)c(-c4ccccc4)c3)c3ccc4ccccc4c3)c2)cc1 ; c1ccc(-c2ccccc2N(c2ccc3ccccc3c2)c2ccc(-c3ccc4sc5cc6ccccc6cc5c4c3)cc2-c2ccccc2)cc1] 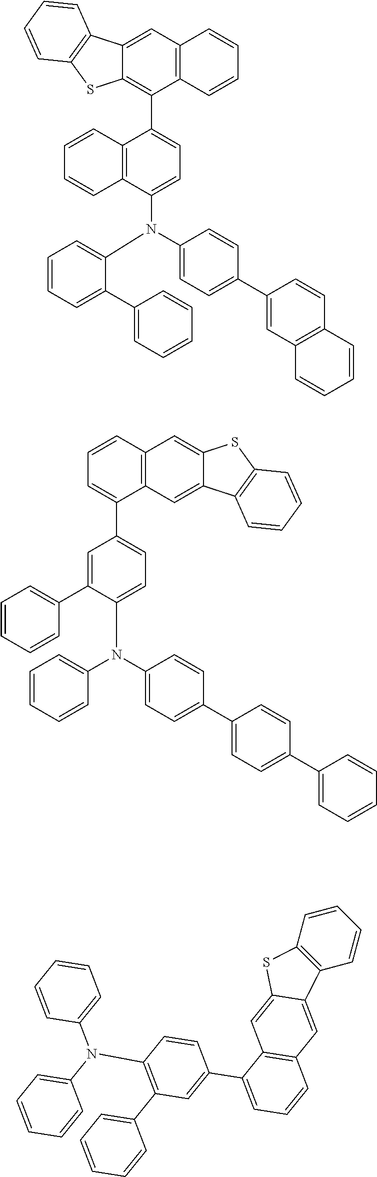 [SMILES: c1ccc(-c2ccccc2N(c2ccc(-c3ccc4ccccc4c3)cc2)c2ccc(-c3c4ccccc4cc4c3sc3ccccc34)c3ccccc23)cc1 ; c1ccc(-c2ccc(-c3ccc(N(c4ccccc4)c4ccc(-c5cccc6cc7sc8ccccc8c7cc56)cc4-c4ccccc4)cc3)cc2)cc1 ; c1ccc(-c2cc(-c3cccc4cc5c(cc34)sc3ccccc35)ccc2N(c2ccccc2)c2ccccc2)cc1] 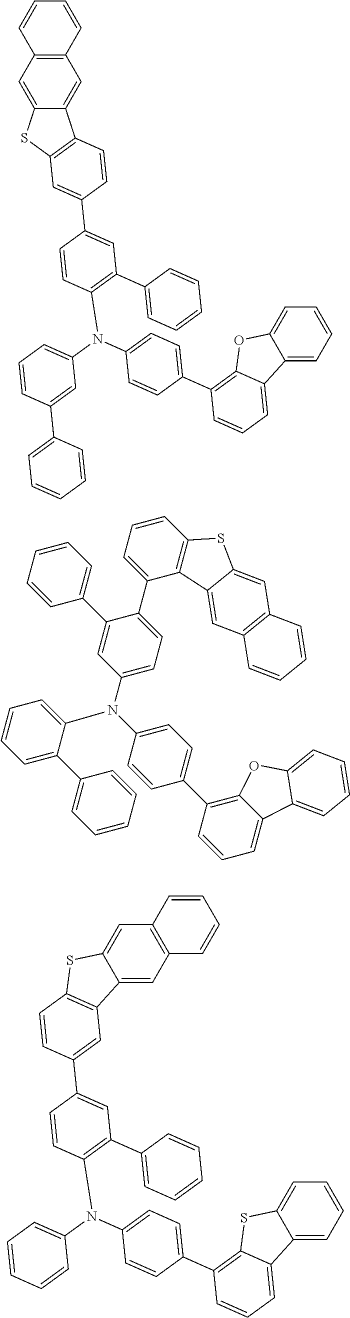 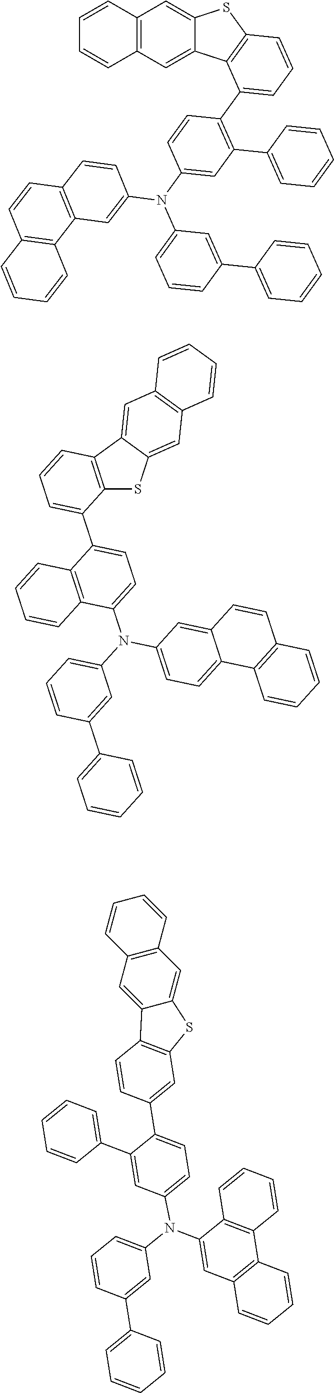 [SMILES: c1ccc(-c2cccc(N(c3ccc(-c4cccc5sc6cc7ccccc7cc6c45)c(-c4ccccc4)c3)c3ccc4ccc5ccccc5c4c3)c2)cc1 ; c1ccc(-c2cccc(N(c3ccc4c(ccc5ccccc54)c3)c3ccc(-c4cccc5c4sc4cc6ccccc6cc45)c4ccccc34)c2)cc1 ; c1ccc(-c2cccc(N(c3ccc(-c4ccc5c(c4)sc4cc6ccccc6cc45)c(-c4ccccc4)c3)c3cc4ccccc4c4ccccc34)c2)cc1] 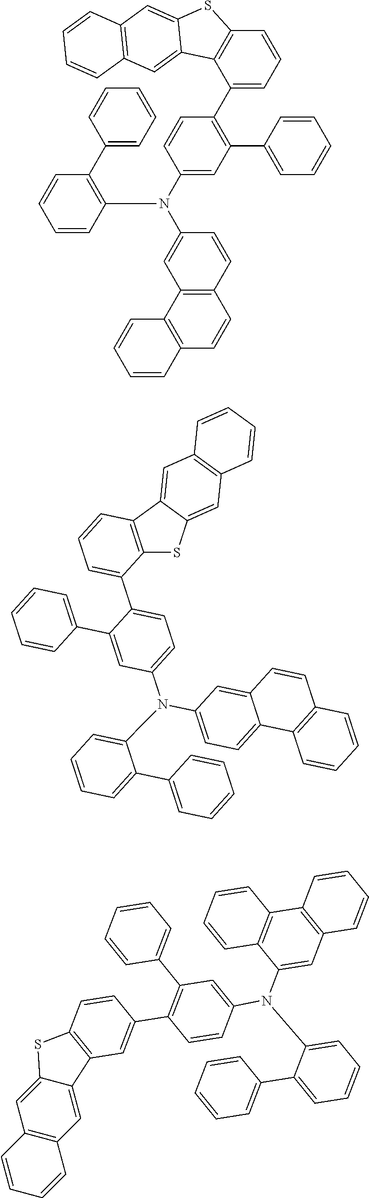 [SMILES: c1ccc(-c2cc(N(c3ccc4ccc5ccccc5c4c3)c3ccccc3-c3ccccc3)ccc2-c2cccc3sc4cc5ccccc5cc4c23)cc1 ; c1ccc(-c2cc(N(c3ccc4c(ccc5ccccc54)c3)c3ccccc3-c3ccccc3)ccc2-c2cccc3c2sc2cc4ccccc4cc23)cc1 ; c1ccc(-c2cc(N(c3ccccc3-c3ccccc3)c3cc4ccccc4c4ccccc34)ccc2-c2ccc3sc4cc5ccccc5cc4c3c2)cc1] 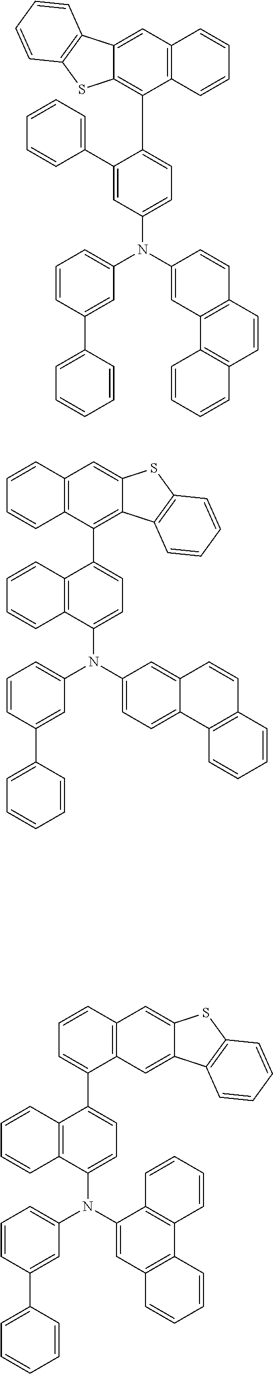 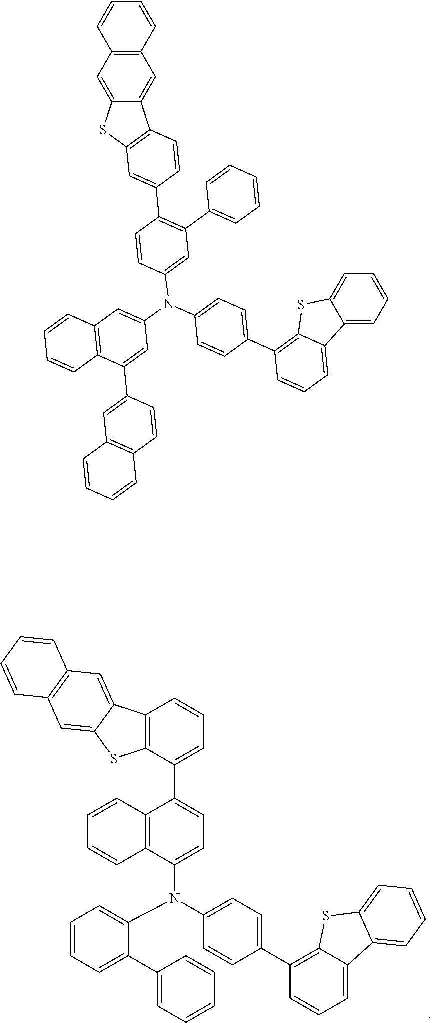 [SMILES: c1ccc(-c2cc(N(c3ccc(-c4cccc5c4sc4ccccc45)cc3)c3cc(-c4ccc5ccccc5c4)c4ccccc4c3)ccc2-c2ccc3c(c2)sc2cc4ccccc4cc23)cc1 ; c1ccc(-c2ccccc2N(c2ccc(-c3cccc4c3sc3ccccc34)cc2)c2ccc(-c3cccc4c3sc3cc5ccccc5cc34)c3ccccc23)cc1]
